(19) United States
(12) Patent Application Publication
(10) Pub. No.: US 2025/0195521 A1
(43) Pub. Date: Jun. 19, 2025

(54) USE OF SOS1 INHIBITORS TO TREAT MALIGNANCIES WITH SHP2 MUTATIONS

(71) Applicant: Revolution Medicines, Inc., Redwood City, CA (US)

(72) Inventors: Ethan AHLER, Redwood City, CA (US); Carlos Ernesto STAHLHUT ESPINOSA, Redwood City, CA (US); David Church MONTGOMERY, Redwood City, CA (US); Elsa QUINTANA, Redwood City, CA (US); David E. WILDES, South San Francisco, CA (US); Grace J. LEE, Redwood City, CA (US); Nataliya TOVBIS SHIFRIN, Redwood City, CA (US)

(21) Appl. No.: 18/043,982

(22) PCT Filed: Sep. 3, 2021
(86) PCT No.: PCT/US2021/048960

§ 371 Date: Mar. 3, 2023

Related U.S. Application Data
(60) Provisional application No. 63/172,791, filed on Apr. 9, 2021.
(60) Provisional application No. 63/135,023, filed on Jan. 8, 2021.
(60) Provisional application No. 63/074,045, filed on Sep. 3, 2020.

(51) Int. Cl. A61K 31/519; A61K 31/341; A61K 31/4045; A61K 31/416; A61K 31/437; A61K 31/443; A61K 31/4439; A61K 31/444; A61K 31/496; A61K 31/4985; A61K 31/4995; A61K 31/517; A61K 31/53; A61K 31/5377; A61K 31/5383; A61K 31/5386; A61K 31/541; A61K 31/55; A61K 31/551; A61K 31/553; A61K 31/675; A61P 35/00

(52) U.S. Cl. CPC A61K 31/519; A61K 31/341; A61K 31/4045; A61K 31/416; A61K 31/437; A61K 31/443; A61K 31/4439; A61K 31/444; A61K 31/496; A61K 31/4985; A61K 31/4995; A61K 31/517; A61K 31/53; A61K 31/5377; A61K 31/5383; A61K 31/5386; A61K 31/541; A61K 31/55; A61K 31/551; A61K 31/553; A61K 31/675; A61P 35/00

(57) ABSTRACT

Methods are provided herein for treating a subject having a disease or disorder associated with cells having a SHP2 mutation, e.g., an activating SHP2 mutation. The SHP2 mutation may cause resistance to a SHP2 inhibitor, e.g., an allosteric SHP2 inhibitor. Methods herein provide administering to the subject a therapeutically effective amount of a SOS1 inhibitor alone or in combination with an additional therapeutic agent.

### CROSS-REFERENCE TO RELATED APPLICATIONS

This application claims priority to U.S. Provisional Application Ser. No. 63/074,045, filed Sep. 3, 2020, the disclosure of which is hereby incorporated by reference as if set forth in its entirety. This application claims priority to U.S. Provisional Application Ser. No. 63/135,023, filed Jan. 8, 2021, the disclosure of which is hereby incorporated by reference as if set forth in its entirety. This application claims priority to U.S. Provisional Application Ser. No. 63/172,791, filed Apr. 9, 2021, the disclosure of which is hereby incorporated by reference as if set forth in its entirety.

### FIELD OF THE DISCLOSURE

The field of the disclosure relates generally to cancer treatment, and more specifically to treatment of cancer associated with SHP2 mutations.

### BACKGROUND OF THE DISCLOSURE

SHP2 is a non-receptor protein tyrosine phosphatase encoded by the PTPN11 gene that contributes to multiple cellular functions including proliferation, differentiation, cell cycle maintenance, and migration. SHP2 is involved in signaling through the RAS-mitogen-activated protein kinase (MAPK), the JAK-STAT pathway, and/or the phosphoinositol 3-kinase-AKT pathway.

SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2.

Mutations in the PTPN11 gene and subsequently in SHP2 have been identified in several human developmental diseases, such as Noonan Syndrome and LEOPARD Syndrome, as well as human cancers, such as juvenile myelomonocytic leukemias, neuroblastoma, melanoma, acute myeloid leukemia, and cancers of the breast, lung and colon. Some of these mutations destabilize the auto-inhibited conformation of SHP2 and promote auto-activation or enhanced growth factor-driven activation of SHP2.

Allosteric SHP2 inhibitors show reduced potency against clinically-relevant SHP2 mutants when the mutant SHP2 is in an activated state. See, e.g., Pádua et al., Nat Commun 9:4507 (2018); LaRochelle et al., Nat Commun 9:4508 (2018). Accordingly, a need exists for treating a disease or disorder associated with cells containing a mutant SHP2.

RAS proteins (KRAS, HRAS and NRAS) play an essential role in various human cancers and are therefore appropriate targets for anticancer therapy. Dysregulation of RAS proteins by activating mutations, overexpression or upstream activation is common in human tumors, and activating mutations in RAS are found in approximately 30% of human cancer. Of the RAS proteins, KRAS is the most frequently mutated and is therefore an important target for cancer therapy. RAS oscillates between GDP-bound “off” and GTP-bound “on” state, facilitated by interplay between a GEF protein (e.g., SOS1), which loads RAS with GTP, and a GAP protein (e.g., NF1), which hydrolyzes GTP, thereby inactivating RAS. Additionally, SHP2 associates with the receptor signaling apparatus and becomes active upon RTK activation, and then promotes RAS activation. Mutations in RAS proteins can lock the protein in the “on” state resulting in a constitutively active signaling pathway that leads to uncontrolled cell growth.

First-in-class covalent inhibitors of the “off” form of KRAS^{G12C} have demonstrated promising anti-tumor activity in cancer patients with KRAS^{G12C} mutations, albeit not in all. Further, therapeutic inhibition of the RAS pathway, although often initially efficacious, can ultimately prove ineffective as it may lead to over-activation of RAS pathway signaling via a number of mechanisms including, e.g., reactivation of the pathway via relief of the negative feedback machineries that naturally operate in these pathways. For example, in various cancers, MEK inhibition results in increased ErbB signaling due to its relief of MEK/ERK-mediated feedback inhibition of RTK activation. As a result, cells that were initially sensitive to such inhibitors may become resistant. Thus, a need exists for methods of effectively inhibiting RAS pathway signaling without inducing activation of resistance mechanisms.

### BRIEF SUMMARY

In some aspects, the present disclosure is directed to a method of treating a subject having a disease or disorder associated with cells having a SHP2 mutation. The method comprises administering to the subject a therapeutically effective amount of a SOS1 inhibitor alone or in combination with an additional therapeutic agent.

In some aspects, the SHP2 mutation induces an activated form of SHP2.

In some aspects, the subject expressed the SHP2 mutation after prior treatment with a SHP2 inhibitor. In some aspects, the subject expressed the SHP2 mutation after prior treatment with an allosteric SHP2 inhibitor.

In some aspects, the method further comprises administering to the subject a therapeutically effective amount of a RAS inhibitor selected from the group consisting of a RAS(ON) inhibitor, a RAS(OFF) inhibitor, and a combination thereof.

### BRIEF DESCRIPTION OF THE DRAWINGS

FIG. 1 is an illustration of the RAS-MAPK pathway. SHP2 activates SOS1, which, in turn, activates RAS, leading to signaling that drives cell growth and survival.

FIG. 2A is a graph depicting activating mutations in SHP2, which reduce sensitivity to inhibition with a SHP2 allosteric inhibitor, RMC-4550. As shown in the graph, cellular sensitivity correlates with energetic magnitude (ΔGₒₚ) of SHP2 activating mutations. Lower ΔGₒₚ indicates stronger activation. FIG. 2B is a table showing the IC₅₀ concentrations of RMC-4550 sufficient to inhibit SHP2 proteins with activating mutations. As shown in the table, strongly activating mutations, such as G503V, require high concentrations of allosteric SHP2 inhibitor to achieve a response. These data were obtained according to the methods disclosed in Example 1.

FIGS. 3A and 3B are graphs showing that SOS1 inhibitors maintain sensitivity in isogenic HEK-293 cell lines carrying a SHP2 E76K mutant. These data were obtained according to the methods disclosed in Example 1.

FIGS. 4A through 4H are graphs showing that activating mutations induce resistance to allosteric SHP2 inhibition but maintain sensitivity to SOS1 inhibitors in isogenic LN229 cell lines. These data were obtained according to the methods disclosed in Example 1.

FIGS. 5A through 5D show that activating mutation G503V induces resistance to allosteric SHP2 inhibition, but maintains sensitivity to SOS1 inhibitors in syngeneic mouse cell lines KLN205 (squamous cell carcinoma) and PAN02 (pancreatic cancer), by a) pERK Alphalisa® assay (FIGS. 5A through 5D graphs), and b) CellTiter-Glo® viability assay (associated Tables). These data were obtained according to the methods disclosed in Example 2.

FIG. 6 is a graph showing SOS1 inhibitors drive tumor growth inhibition in vivo in the context of activating SHP2 mutation G503V in syngeneic mouse cell line KLN205 (squamous cell carcinoma) in immunocompetent mice. These data were obtained according to the methods disclosed in Example 2.

FIG. 7 is a graph showing SOS1 inhibitors drive tumor growth inhibition in vivo in the context of activating SHP2 mutation G503V in syngeneic mouse cell line PAN02 in immunocompetent mice. These data were obtained according to the methods disclosed in Example 2.

FIGS. 8A through 8D are graphs showing LN229 cells with various SHP2 mutational variants confirmed to be sensitive to inhibition in non-targeting control with partial depth of inhibition, rescued from inhibition by BI-3406 by knockdown of SOS1, and sensitized by knockdown of SOS2 to restore full depth of inhibition. These data were obtained according to the methods disclosed in Example 3.

FIG. 9 is a graph showing that SHP2 mutants demonstrated significant reduction in basal pERK after SOS1 knockdown, and levels of pERK reduction correlate with biochemical potency for activated SHP2 variant.

FIG. 10 is a graph showing measured specific activity of SHP2 on DiFMUP as a function of [RMC-4550] and [SIRPA¹]. These data were obtained according to the methods disclosed in Example 3.

FIG. 11 is a graph showing fit of data from FIG. 10 to the equation shown in Example 4 for wild type SHP2. These data were obtained according to the methods disclosed in Example 4.

FIG. 12 is a graph showing fit of data from FIG. 10 to the equation shown in Example 4 for mutant SHP2 A⁷²S. These data were obtained according to the methods disclosed in Example 4.

FIG. 13 is a graph showing fit of data from FIG. 10 to the equation shown in Example 4 for mutant SHP2 E69K. These data were obtained according to the methods disclosed in Example 4.

FIG. 14 is a graph showing fit of data from FIG. 10 to the equation shown in Example 4 for mutant SHP2 G503V. These data were obtained according to the methods disclosed in Example 4.

FIG. 15 is a graph showing SOS1 inhibitors drive tumor growth inhibition in vivo in the context of activating SHP2 mutation G503V in syngeneic mouse cell line PAN02 in immunocompetent mice. These data were obtained according to the methods disclosed in Example 5.

FIG. 16 is a graph showing SOS1 inhibitors drive tumor growth inhibition in vivo in the context of activating SHP2 mutation A⁷²S in syngeneic mouse cell line LN229 CDX in immunocompromised mice. These data were obtained according to the methods disclosed in Example 5.

FIGS. 17A and 17B are graphs showing the additive effect of SOS1 inhibitor Compound SOS1-(B) and RAS inhibitor Compound RAS-(E) in cell lines having both SHP2 and KRAS mutations. These data were obtained according to the methods disclosed in Example 6.

FIG. 18 is a Loewe 3D response surface plot showing the in vitro combination effect of SOS1 inhibitor Compound SOS1-(A) (also called RMC-0331) and RAS^{MULTI} (ON) inhibitor Compound RAS-(D) observed in Pan02 cells. A synergy score >5 at any point on the plot indicates a positive interaction between the two compounds. These data were obtained according to the methods disclosed in Example 7.

FIG. 19 is a Loewe 3D response surface plot showing the in vitro combination effect of SOS1 inhibitor Compound SOS1-(A) (also called RMC-0331) and RAS^{MULTI} (ON) inhibitor Compound RAS-(D) observed in KLN205 cells (squamous cell carcinoma). A synergy score >5 at any point on the plot indicates a positive interaction between the two compounds. These data were obtained according to the methods disclosed in Example 7.

FIG. 20A is a graph depicting activating mutations in SHP2, which reduce sensitivity to inhibition with a SHP2 allosteric inhibitor (RMC-4550) but maintain sensitivity to a SOS1 inhibitor (Compound SOS1-(A)). Lower ΔGₒₚ indicates stronger activation, and cellular sensitivity to SHP2 inhibition correlates with energetic magnitude (ΔGₒₚ) of SHP2 activating mutations. In contrast, cellular sensitivity to SOS1 inhibition is maintained across all SHP2 mutations tested. FIG. 20B is a table showing the IC₅₀ concentrations of RMC-4550 and Compound SOS1-(A) in cells with different SHP2 mutations. As shown in the table, strongly activating mutations, such as E76K, require high concentrations of allosteric SHP2 inhibitor to achieve a response, but relatively low concentrations of SOS1 inhibitor are required to achieve a response across all SHP2 mutations tested. These data were obtained according to the methods disclosed in Example 1.

FIG. 21 is a graph showing SOS1 inhibitors drive tumor growth inhibition in vivo in the context of activating SHP2 mutation E76K in syngeneic mouse cell line LN229.E76K in immunocompromised mice. These data were obtained according to the methods disclosed in Example 8.

FIG. 22 is a graph showing SOS1 inhibitors alone and in combination drive tumor growth inhibition in vivo in the context of activating SHP2 mutation G503V in syngeneic mouse cell line PAN02 in immunocompetent mice. These data were obtained according to the methods disclosed in Example 9.

FIG. 23 is a graph showing SOS1 inhibitors alone and in combination drive tumor growth inhibition in vivo in the context of activating SHP2 mutation G503V in syngeneic mouse cell line PAN02 in immunocompetent mice. These data were obtained according to the methods disclosed in Example 10.

FIG. 24 is a graph showing SOS1 inhibitors alone and in combination drive tumor growth inhibition in vivo in the context of activating SHP2 mutation E76K in syngeneic mouse cell line LN229.E76K in immunocompromised mice. These data were obtained according to the methods disclosed in Example 11.

FIG. 25 is a graph showing SOS1 inhibitors alone and in combination drive tumor growth inhibition in vivo in the context of activating SHP2 mutation G503V in syngeneic mouse cell line PAN02 in immunocompetent mice. These data were obtained according to the methods disclosed in Example 12.

FIGS. 26A, 26B, 26C, 26D, 26E, and 26F are graphs and tables showing SOS1 inhibitors alone and in combination drive tumor growth inhibition in vivo in the context of activating SHP2 mutation G503V in syngeneic mouse cell line PAN02 in immunocompetent mice. These data were obtained according to the methods disclosed in Example 13.

### DETAILED DESCRIPTION OF THE DISCLOSURE

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which the present disclosure belongs. All patents and publications cited in this specification are incorporated herein by reference in their entireties.

### Terms

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Pharmaceutically acceptable salts of compounds disclosed herein are contemplated by the present invention. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “therapeutically effective amount” when used in connection with a compound is an amount effective for treating or preventing a disease in a subject as described herein.

The disclosure also includes pharmaceutical compositions comprising an effective amount of a disclosed compound and a pharmaceutically acceptable carrier. The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The terms “inhibiting” and “reducing,” or any variation of these terms, includes any measurable or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., SOS1: Ras-family protein binding activity) compared to normal.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

The term “sample” or “biological sample,” as used herein, refers to a sample obtained from a subject, e.g., a human subject or a patient, which may be tested for a particular molecule, for example wild type. Samples may include, but are not limited to, biopsies, tissues, cells, buccal swab sample, body fluids, including blood, serum, plasma, urine, saliva, cerebral spinal fluid, tears, pleural fluid and the like.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein. In some embodiments, the inhibitor can be nucleic acid molecules including, but not limited to, siRNA that reduce the amount of functional protein in a cell. Accordingly, compounds said to be “capable of inhibiting” a particular protein, e.g., SHP2 or SOS1, comprise any such inhibitor.

The term “mutation” as used herein indicates any modification of a nucleic acid and/or polypeptide which results in an altered nucleic acid or polypeptide. The term “mutation” may include, for example, point mutations, deletions of a single or multiple residues in a polynucleotide, or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

The term “SHP2” means “Src Homology 2 domain-containing protein tyrosine phosphatase 2” and is also known as SH-PTP2, SH-PTP3, Syp, PTPID, PTP2C, SAP-2 or PTPN11. SHP2 is a non-receptor protein tyrosine phosphatase encoded by the PTPN11 gene that contributes to multiple cellular functions including proliferation, differentiation, cell cycle maintenance and migration. SHP2 is involved in signaling through the RAS-mitogen-activated protein kinase (MAPK), the JAK-STAT and/or the phosphoinositol 3-kinase-AKT pathways. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2.

The term “allosteric SHP2 inhibitor” means an agent (e.g., a small-molecule compound (e.g., less than 750 Da)) capable of inhibiting SHP2 through binding to SHP2 at a site other than the active site of the enzyme.

The term “inhibitor-resistant mutation” when used in reference to a SHP2 mutation, means a SHP2 mutation that renders a SHP2 polypeptide refractory or resistant to inhibition with a SHP2 inhibitor. Thus, in some embodiments, an inhibitor-resistant mutation in a SHP2 polypeptide decreases the inhibitory effect that a SHP2 inhibitor has on the SHP2 polypeptide as compared to the effect the inhibitor has on a similar SHP2 polypeptide differing only in the absence of the inhibitor-resistant mutation. Such activity may be measured using any suitable activity assay known in the art or disclosed herein. In some embodiments, an inhibitor-resistant mutation in a SHP2 polypeptide abolishes all detectable inhibitory effects that a SHP2 inhibitor has on the activity of the SHP2 polypeptide, wherein the inhibitor has detectable inhibitory efficacy on a similar SHP2 polypeptide differing only in the absence of the inhibitor-resistant mutation. Such inhibitor-resistant mutations include, without limitation, mutations that destabilize the auto-inhibited conformation of SHP2. An inhibitor-resistant mutation may be an allosteric inhibitor-resistant mutation.

As used herein, the term “activating SHP2 mutation” or “activated mutation of SHP2” or similar refers to a mutation of SHP2 that destabilizes the auto-inhibited conformation of SHP2, as measured by the free energy of opening (ΔGₒₚ) of the mutation. Wild-type SHP2 has a ΔGₒₚ of 2.8 kcal/mol. Values of ΔGₒₚ below 2.8 in mutant SHP2 indicate activation, with lower values indicating stronger activation. A weakly activating SHP2 mutant is defined as one with a ΔGₒₚ not more than 1.5 kcal/mol below wild type SHP2. A moderately activating SHP2 mutant has a ΔGₒₚ between 1.5 kcal/mol and 2.24 kcal/mol below wild-type, and a strongly activating SHP2 mutation has a ΔGₒₚ more than 2.24 kcal/mol below wild type. Methods of measuring ΔGₒₚ are provided in Example 4.

The term “allosteric inhibitor-resistant mutation” when used in reference to a SHP2 mutation, means a SHP2 mutation that renders a SHP2 polypeptide refractory or resistant to inhibition with a SHP2 allosteric inhibitor. Thus, in some embodiments, an allosteric inhibitor-resistant mutation in a SHP2 polypeptide decreases the inhibitory effect that a SHP2 allosteric inhibitor has on the SHP2 polypeptide as compared to the effect the inhibitor has on a similar SHP2 polypeptide differing only in the absence of the allosteric inhibitor-resistant mutation. Such activity may be measured using any suitable activity assay known in the art or disclosed herein. In some embodiments, an allosteric inhibitor-resistant mutation in a SHP2 polypeptide abolishes all detectable inhibitory effects that a SHP2 allosteric inhibitor has on the activity of the SHP2 polypeptide, wherein the inhibitor has detectable inhibitory efficacy on a similar SHP2 polypeptide differing only in the absence of the allosteric inhibitor-resistant mutation. Such allosteric inhibitor-resistant mutations include, without limitation, mutations that destabilize the auto-inhibited conformation of SHP2. In some embodiments, the allosteric inhibitor-resistant mutation is a SHP2 mutation is a mutation as described herein.

The term “SOS” (e.g., a “SOS mutation”) refers to SOS genes, which are known in the art to include RAS guanine nucleotide exchange factor proteins that are activated by receptor tyrosine kinases to promote GTP loading of RAS and signaling. The term SOS includes all SOS homologs that promotes the exchange of Ras-bound GDP by GTP. In particular embodiments, SOS refers specifically to “son of sevenless homolog 1” (“SOS1”). SOS1 is critically involved in the activation of RAS-family protein signaling in cancer via mechanisms other than mutations in RAS-family proteins. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, PDGFR-A/B, FGFR^{1/2/3}, IGF1 R, INSR, ALK, ROS, TrkA, TrkB, TrkC, RET, c-MET, VEGFR^{1/2/3}, AXL) (Pierre et al., Biochem. Pharmacol., 2011, 82 (9): 1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275 (8): 5966-75). This localization of SOS1 to the plasma membrane, proximal to RAS-family proteins, enables SOS1 to promote RAS-family protein activation. SOS1-activation of RAS-family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6 (3): 268-74). SOS1 is also a GEF for the activation of the GTPases RAC1 (Ras-related C3 botulinum toxin substrate 1) (Innocenti et al., J. Cell Biol., 2002, 156 (1): 125-36). RAC1, like RAS-family proteins, is implicated in the pathogenesis of a variety of human cancers and other diseases (Bid et al., Mol. Cancer Ther. 2013, 12 (10): 1925-34). Son of sevenless 2 (SOS2), a homolog of SOS1 in mammalian cells, also acts as a GEF for the activation of RAS-family proteins (Pierre et al., Biochem. Pharmacol., 2011, 82 (9): 1049-56; Buday et al., Biochim. Biophys. Acta., 2008, 1786 (2): 178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19 (4): 642-54), systemic conditional SOS1 knockout adult mice are viable (Baltanas et al., Mol. Cell. Biol., 2013, 33 (22): 4562-78). SOS2 gene targeting did not result in any overt phenotype in mice (Esteban et al., Mol. Cell. Biol., 2000, 20 (17): 6410-3). In contrast, double SOS1 and SOS2 knockout leads to rapid lethality in adult mice (Baltanas et al., Mol. Cell. Biol., 2013, 33 (22): 4562-78). These published data suggest that selective targeting of individual SOS isoforms (e.g., selective SOS1 targeting) may be adequately tolerated to achieve a therapeutic index between SOS1/RAS-family protein driven cancers (or other SOS1/RAS-family protein pathologies) and normal cells and tissues. Selective pharmacological inhibition of the binding of the catalytic site of SOS1 to RAS-family proteins is expected to prevent SOS1-mediated activation of RAS-family proteins to the GTP-bound form. Such SOS1 inhibitor compounds are be expected to consequently inhibit signaling in cells downstream of RAS-family proteins (e.g., ERK phosphorylation). In cancer cells associated with dependence on RAS-family proteins (e.g., KRAS mutant cancer cell lines), SOS1 inhibitor compounds are be expected to deliver anti-cancer efficacy (e.g., inhibition of proliferation, survival, metastasis, etc.). High potency towards inhibition of SOS1: RAS-family protein binding (nanomolar level IC₅₀ values) and ERK phosphorylation in cells (nanomolar level IC₅₀ values) are desirable characteristics for a SOS1 inhibitor compound. Furthermore, a desirable characteristic of a SOS1 inhibitor compound would be the selective inhibition of SOS1 over SOS2. This conclusion is based on the viable phenotype of SOS1 knockout mice and lethality of SOS1/SOS2 double knockout mice, as described above.

As used herein, a “SOS1 inhibitor” refers to any agent, (e.g., a small molecule (e.g., less than 750 Da)) capable of inhibiting SOS1. SOS1 inhibitors can include selective SOS1 inhibitors and inhibitors that also inhibit other proteins. In some embodiments, SOS1 inhibitors may also inhibit SOS2, with a selectivity ratio less than 10-fold for inhibition of SOS1 relative to SOS2. In some embodiments, SOS1 inhibitors will selectively inhibit SOS1, with a selectivity ratio greater of at least about 10-fold, such as greater than at least about 30-fold, for inhibition of SOS1 relative to SOS2.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The terms “RAS inhibitor” and “inhibitor of [a] RAS” are used interchangeably to refer to any inhibitor that targets a RAS protein. In various embodiments, these terms include RAS(OFF) and RAS(ON) inhibitors such as, e.g., the KRAS(OFF) and KRAS(ON) inhibitors. A RAS inhibitor may be MRTX1133, or a pharmaceutically acceptable salt, solvate, isomer (e.g., stereoisomer), prodrug, or tautomer thereof. The term “RAS(OFF) inhibitor” refers to any inhibitor that binds to a RAS protein in its GDP-bound “OFF” position. The term “RAS(ON) inhibitor” refers to any inhibitor that binds to a RAS protein in its GTP-bound “ON” position.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). The term “KRAS(ON) inhibitor” refers to any inhibitor that binds to KRAS in its GTP-bound “ON” position.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

The term “KRAS(OFF) inhibitor” refers to any inhibitor that binds to KRAS in its GDP-bound “OFF” position. Reference to the term KRAS(OFF) inhibitor includes AMG 510 and MRTX849, or a pharmaceutically acceptable salt, solvate, isomer (e.g., stereoisomer), prodrug, or tautomer thereof. In some embodiments, reference to the term KRAS(OFF) inhibitor includes any such KRAS(OFF) inhibitor disclosed in any one of the following patent applications: WO 2020118066, WO 2020113071, WO 2020106647, WO 2020106640, WO 2020102730, WO 2020101736, WO 2020097537, WO 2020086739, WO 2020018282, WO 2020050890, WO 2020047192, WO 2020035031, WO 2020033413, WO 2020028706, WO 2019241157, WO 2019234405, WO 2019232419, WO 2019227040, WO 2019217933, WO 2019217691, WO 2019217307, WO 2019215203, WO 2019213526, WO 2019213516, WO 2019204442, WO 2019204449, WO 2019204505, WO 2019155399, WO 2019150305, WO 2019137985, WO 2019110751, WO 2019099524, WO 2019055540, WO 2019051291, WO 2018237084, WO 2018218070, WO 2018217651, WO 2018218071, WO 2018218069, WO 2018212774, WO 2018206539, WO 2018195439, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2018011351, WO 2018005678, WO 2017201161, WO 20171937370, WO 2017172979, WO 2017112777, WO 2017106520, WO 2017096045, WO 2017100546, WO 2017087528, WO 2017079864, WO 2017058807, WO 2017058805, WO 2017058728, WO 2017058902, WO 2017058792, WO 2017058768, WO 2017058915, WO 2017015562, WO 2016179558, WO 2016176338, WO 2016168540, WO 2016164675, WO 2016100546, WO 2016049568, WO 2016049524, WO 2015054572, WO 2014152588, WO 2014143659 and WO 2013155223, or a pharmaceutically acceptable salt, solvate, isomer (e.g., stereoisomer), prodrug, or tautomer thereof, each of which are incorporated herein by reference in its entirety.

As used herein, the term “RAS(ON) MULTI inhibitor” refers to a RAS(ON) inhibitor of at least 3 RAS variants with missense mutations at one of the following positions: 12, 13, 59, 61, or 146. In some embodiments, a RAS(ON) MULTI inhibitor refers to a RAS(ON) inhibitor of at least 3 RAS variants with missense mutations at one of the following positions: 12, 13, and 61.

In any embodiment herein regarding a RAS(OFF) inhibitor, such RAS(OFF) inhibitor may be substituted by a RAS inhibitor disclosed in the following patent publication: WO 2021041671, which is incorporated herein by reference in its entirety. In some embodiments, such a substituted RAS inhibitor is MRTX1133, or a pharmaceutically acceptable salt, solvate, isomer (e.g., stereoisomer), prodrug, or tautomer thereof.

Exemplary RAS(OFF) inhibitors include the following, without limitation:

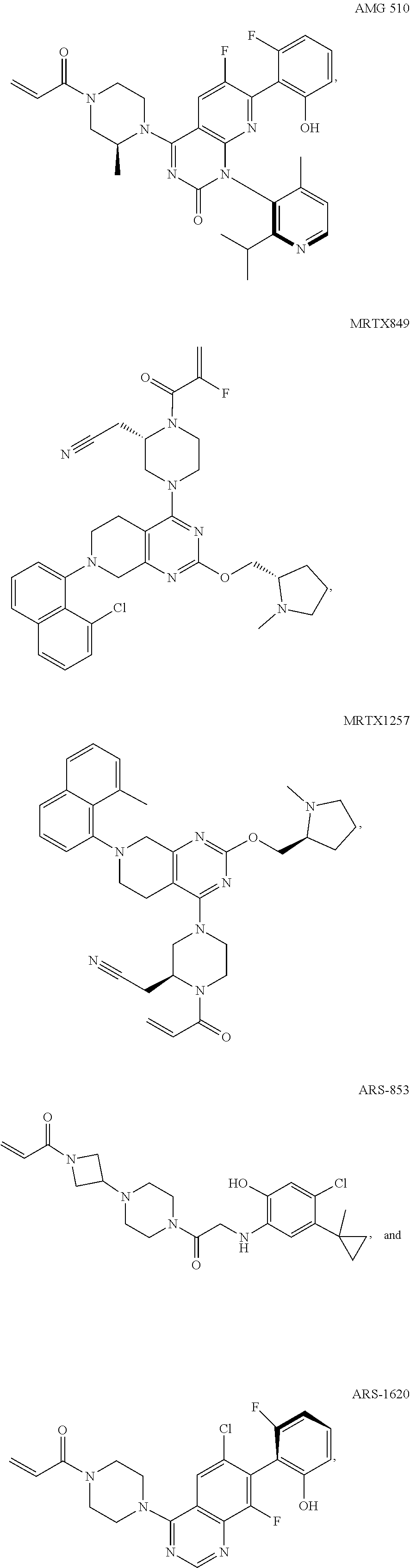

or a pharmaceutically acceptable salt, solvate, isomer (e.g., stereoisomer), prodrug, or tautomer thereof.

Reference to a “subtype” of a cell (e.g., a KRAS^{G12C} subtype, a KRAS^{G12S} subtype, a KRAS^{G12D} subtype, a KRAS^{G12V} subtype) means that the cell contains a gene mutation encoding a change in the protein of the type indicated. For example, a cell classified as a “KRAS^{G12C} subtype” contains at least one KRAS allele that encodes an amino acid substitution of cysteine for glycine at position 12 (^{G12C}); and, similarly, other cells of a particular subtype (e.g., KRAS^{G12D}, KRAS^{G12S} and KRAS^{G12V} subtypes) contain at least one allele with the indicated mutation (e.g., a KRAS^{G12D} mutation, a KRAS^{G12S} mutation or a KRAS^{G12V} mutation, respectively). Unless otherwise noted, all amino acid position substitutions referenced herein (such as, e.g., “^{G12C}” in KRAS^{G12C}) correspond to substitutions in the human version of the referenced protein, i.e., KRAS^{G12C} refers to a G→C substitution in position 12 of human KRAS.

As used herein, “RASopathies” are a group of genetic conditions caused by changes in genes that are part of the RAS pathway. See, e.g., Rauen, Ann Rev Genomics Human Genetics, 14:355 (2013). Non-limiting examples include Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

The term “monotherapy” refers to a method of treatment comprising administering to a subject a single therapeutic agent, optionally as a pharmaceutical composition. For example, a monotherapy may comprise administration of a pharmaceutical composition comprising a therapeutic agent and one or more pharmaceutically acceptable carrier, excipient, diluent, and/or surfactant. The therapeutic agent may be administered in an effective amount. The therapeutic agent may be administered in a therapeutically effective amount.

The term “combination therapy” refers to a method of treatment comprising administering to a subject at least two therapeutic agents, optionally as one or more pharmaceutical compositions. For example, a combination therapy may comprise administration of a single pharmaceutical composition comprising at least two therapeutic agents and one or more pharmaceutically acceptable carrier, excipient, diluent, and/or surfactant. A combination therapy may comprise administration of two or more pharmaceutical compositions, each composition comprising one or more therapeutic agent and one or more pharmaceutically acceptable carrier, excipient, diluent, and/or surfactant. In various embodiments, at least one of the therapeutic agents is a SOS1 inhibitor. In various embodiments, at least one of the therapeutic agents is a RAS inhibitor. The two agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due an additive or synergistic effect of combining the two or more therapeutics.

### Methods of Treatment

Disruption of the RAS/MAPK signaling pathway is a common driver of abnormal growth and proliferation in many types of cancer and has also been implicated in developmental diseases such as Noonan Syndrome. Oncogenic hyper-activation of this pathway can occur through alterations in the levels of active GTP-bound RAS and inactive GDP-bound RAS, such as mutations resulting in disruption of RAS guanine nucleotide exchange factors (GEFs) and GTPase-activating proteins (GAPs). SHP2 is a non-receptor protein tyrosine phosphatase encoded by the PTPN11 gene that functions upstream of RAS. SHP2 can regulate RAS signaling through activation of SOS1, a GEF that converts inactive RAS-GDP to RAS-GTP. The development of inhibitors targeting either SHP2 or SOS1 is an emerging and attractive approach toward treatment of RAS-driven cancers, and several such candidates are currently undergoing clinical trials.

According to some embodiments of the present invention, a subject having a disease or disorder associated with cells having a SHP2 mutation is treated by administering to the subject a therapeutically effective amount of a SOS1 inhibitor. In some embodiments, the SHP2 mutation induces an activated form of SHP2. In some embodiments, the subject expressed the SHP2 mutation after prior treatment with a SHP2 inhibitor. In some embodiments, the subject expressed the SHP2 mutation after prior treatment with an allosteric SHP2 inhibitor.

In some embodiments, the method further comprises administering to the subject a therapeutically effective amount of a RAS inhibitor selected from the group consisting of a RAS(ON) inhibitor, a RAS(OFF) inhibitor, MRTX1133, and a combination thereof. In some embodiments, the RAS inhibitor targets a wild-type RAS protein. In some embodiments, the RAS protein is KRAS. In some embodiments, the RAS inhibitor targets a RAS protein mutation. In some embodiments, the RAS protein mutation is at a position selected from the group consisting of G12, G13, Q61, A¹⁴⁶, K117, L19, Q22, V14, A⁵⁹, and a combination thereof. In some embodiments, the mutation is at a position selected from the group consisting of G12, G13, and Q61. In some embodiments, the mutation is selected from the group consisting of G12C, G12D, G12A, G12S, G12V, G13C, G13D, Q61K, and Q61L.

### SHP2 Mutations

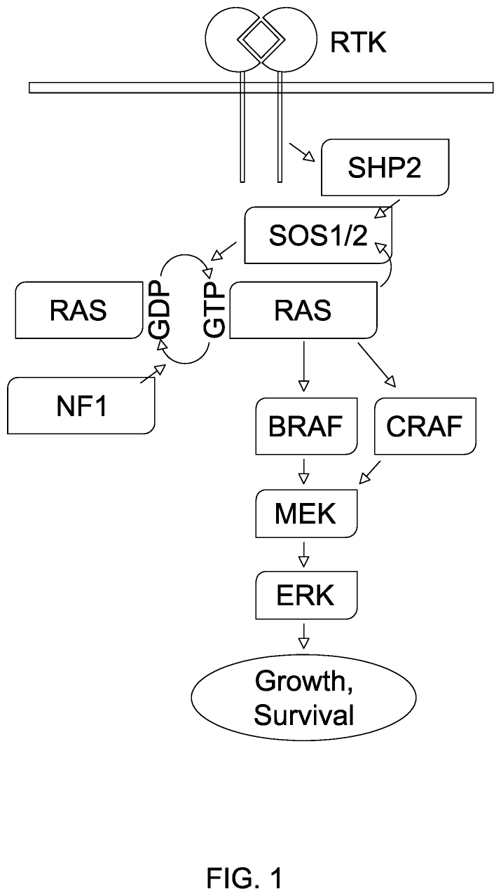

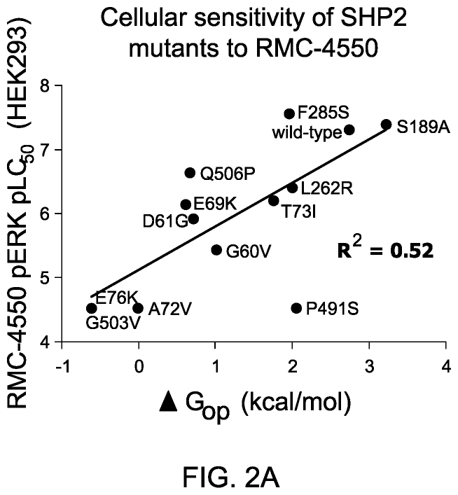

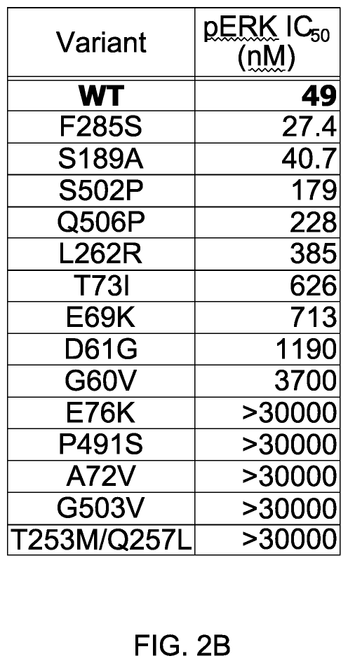

Potent and selective allosteric SHP2 inhibitors, such as RMC-4550, have proven effective in vitro and in vivo across a wide range of histotypes and genotypes, at disrupting RAS-MAPK signaling, suppressing cell proliferation, and inducing tumor growth inhibition. Allosteric SHP2 inhibitors are effective in preclinical models of cancers driven by the RAS/MAPK signaling pathway, in part because they block activation of the RAS GEFs SOS1 and SOS2. See FIG. 1, which illustrates the RAS-MAPK pathway. However, SHP2 inhibitors, including allosteric SHP2 inhibitors, have previously been demonstrated to exhibit significantly reduced efficacy against a spectrum of specific clinically relevant mutations in SHP2 that induce an activated form of the SHP2 protein. Mutations can occur in SHP2 that, to varying degrees, activate signaling and reduce sensitivity to allosteric inhibitors. These mutations may arise during tumor development as drivers or be acquired as resistance mutations in response to treatment with allosteric SHP2 inhibitors. See FIGS. 2A and 2B, which depict several activating mutations in SHP2 proteins. In addition, patients treated with SHP2 inhibitors, including allosteric SHP2 inhibitors, may develop tumors with somatic SHP2 mutations, inducing pathway reactivation and drug resistance. Therefore, there exists an unmet medical need for alternative treatments for cancers associated with activating mutations of SHP2 protein.

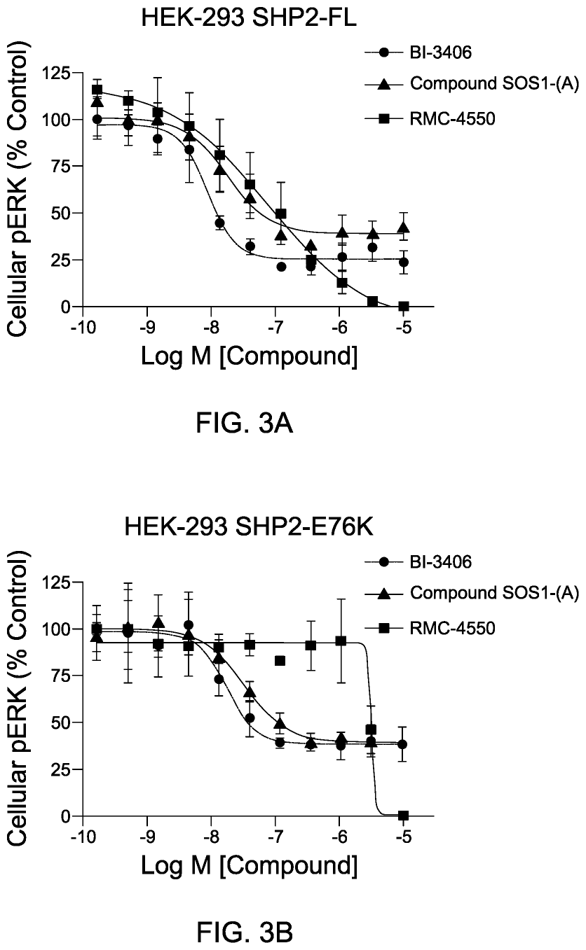

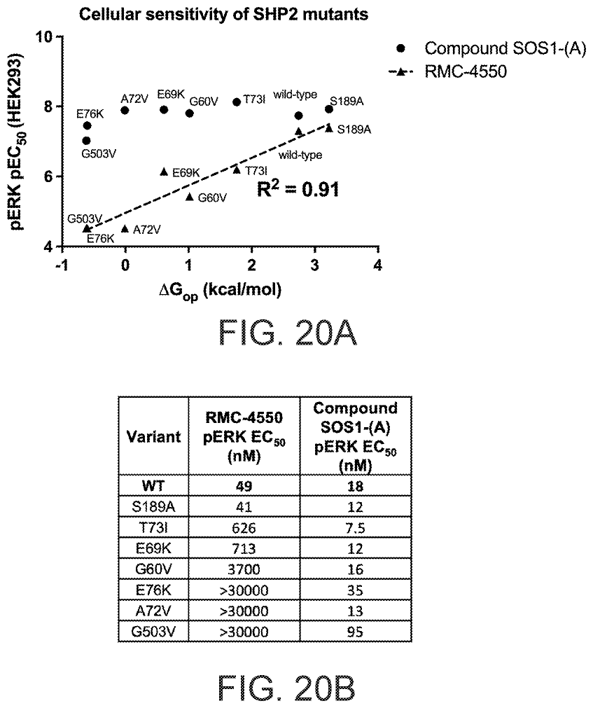

Potent SOS1 inhibitors, including selective SOS1 inhibitors, have also been developed and have more recently shown preclinical promise as an alternative treatment for RAS/MAPK pathway-driven cancers. The first clinical trial involving a SOS1 inhibitor has recently begun. SOS1 inhibitors, which act directly downstream of SHP2, would represent an attractive alternative treatment in this regard if they maintain or show improved efficacy in the context of activated mutant SHP2. The present disclosure provides evidence in support of this concept, showing that in contrast to SHP2 inhibitors, e.g., allosteric SHP2 inhibitors, SOS1 inhibition is efficacious in vitro and in vivo in cells or tissue with activating SHP2 mutations, even when SHP2 allosteric inhibitors are not effective. See FIGS. 3A, 3B, 4A through 4H, and 20A, and 20B.

Unexpectedly, genetic knockdown of SOS1 appears to induce a greater reduction in pathway activity in cells with activated SHP2, indicating that cancer cells harboring activating SHP2 mutations are uniquely dependent on SOS1. Experimental results showed that all cells are sensitive to simultaneous knockdown of SOS1 and SOS2, but unexpectedly, a trend is seen of greater effect of SOS1 knockdown in cells with more strongly activating SHP2 mutations. In contrast, SOS2 knockdown has only a small and similar effect in all cell lines. This suggests that strongly activating SHP2 mutations increase dependence on SOS1, and activating SHP2 mutations may therefore increase sensitivity to SOS1 inhibitors such as BI-3406, BI-1701963, and Compound SOS1-(A) (also called RMC-0331), or a pharmaceutically acceptable salt, solvate, isomer (e.g., stereoisomer), prodrug, or tautomer thereof. This observation provides the basis for a therapeutic strategy whereby cancer patients with mutations in SHP2, e.g., activating SHP2 mutations, either present initially or arising in response to therapy with SHP2 inhibitors, e.g., allosteric SHP2 inhibitors, may be effectively treated with SOS1 inhibitors.

According to some embodiments of the present invention, a method is provided for treating a subject having a SHP2 mutation, e.g., a SHP2 mutation that induces an activated form of SHP2. These mutations act by destabilizing an auto-inhibited conformation of SHP2. Different activating mutations destabilize this conformation to different degrees, which can be expressed quantitatively as the free energy of opening (ΔGₒₚ) of the mutation. Wild-type SHP2 has a ΔGₒₚ of 2.8 kcal/mol. Values of ΔGₒₚ below 2.8 in mutant SHP2 indicate activation, with lower values indicating stronger activation. A weakly activating mutant is defined as one with a ΔGₒₚ not more than 1.5 kcal/mol below wild type SHP2. A moderately activating mutant has a ΔGₒₚ between 1.5 kcal/mol and 2.24 kcal/mol below wild-type. A strongly activating mutation has a ΔGₒₚ more than 2.24 kcal/mol below wild type. See FIG. 2A, which is a graph correlating the RMC-4550 pERK IC₅₀ as a function of ΔGₒₚ. FIG. 2B is a table showing the pERK IC₅₀ values for RMC-4550 in a variety of activating mutations of SHP2 protein. The following Table 1 summarizes the model parameters for SHP2 and certain mutants.

TABLE 1
Summary of model parameters for SHP2 and mutants

| | ΔGₒₚ | ΔG_{peptide} | ΔGᵢ | Aₒₚ |
|---|---|---|---|---|
| Variant | (kcal/mol) | (kcal/mol) | (kcal/mol) | (mRFU/s/nM) |
| wild-type | 2.74 | −10.78 | −11.87 | 35 |
| G60V | 1.01 | −11.48 | −10.38 | 31.77 |
| D61G | 0.72 | −10.55 | −11.94 | 34.51 |
| D61V | 0.37 | −11.39 | −11.23 | 33.39 |
| E69K | 0.61 | −11.54 | −11.59 | 30.25 |
| A72S | 2.03 | −11.07 | −11.56 | 28.24 |
| A72T | 0.31 | −10.72 | −11.18 | 33.21 |
| A72V | −0.01 | −11.05 | −10.62 | 31.84 |
| T73I | 1.76 | −11.69 | −11.56 | 38.4 |
| E76A | 0.41 | −11.26 | −11.51 | 32.1 |
| E76G | 0.53 | −11.14 | −11.45 | 35.64 |
| E76K | −0.60 | −10.91 | −10.92 | 29.51 |
| E76Q | 1.24 | −11.33 | −11.91 | 30.83 |
| S189A | 3.22 | −10.52 | −11.95 | 35 |
| L262R | 2.00 | −10.94 | −11.85 | 37.72 |
| F285S | 1.97 | −10.77 | −11.94 | 19.81 |
| N308D | 1.89 | −11.12 | −11.96 | 34.42 |
| T468M | 3.63 | −10.52 | −11.11 | 5 |
| P491S | 2.06 | −11.13 | −9.13 | 35.67 |
| G503V | −0.62 | −10.57 | −9.31 | 53.97 |
| Q506P | 0.67 | −10.38 | −11.19 | 2.679 |
| T507K | 1.97 | −10.53 | −11.23 | 30.3 |

As shown in FIG. 2A and Table 1, G503V, a mutation resulting in the lowest free energy of opening (ΔGₒₚ), is a particularly activating mutation of SHP2. This is further shown, for example, by the pERK IC₅₀ value of >30,000 nM for G503V in FIG. 2B.

Accordingly, in some embodiments, a SHP2 mutation may occur at one or more of the following positions: G60, D61, E69, A⁷², T73, E76, S189, L262, F285, N308, T468, P491, S502, G503, Q506, T507, T253 or Q257. In some embodiments, a mutation is one or more of the following: G60V, D61G, D61V, E69K, A⁷²S, A⁷²T, A⁷²V, T73I, E76A, E76G, E76K, E76Q, S189A, L262R, F285S, N308D, T468M, P491S, S502P, G503V, Q506P, T507K, T253M/Q257L, and a combination thereof.

Moreover, in some embodiments, a SHP2 mutation is at a position that occurs with a frequency in subjects greater than an alteration prevalence greater than 0.05%. In some embodiments, a SHP2 mutation is at a position selected from the group consisting of T52, I56, G60, D61, Y⁶², Y⁶³, E69, K70, A⁷², T73, E76, E123, E139, Y¹⁹⁷, S189, T253, Q257, L261, L262, R²⁶⁵, F285, N308, V428, A⁴⁶¹, T468, P491, S502, G503, M504, Q506, Q510, T507, and a combination thereof. In some embodiments, a SHP2 mutation is at a position selected from the group consisting of A⁷², E76 and G503, and a combination thereof. In some embodiments, a SHP2 mutation is selected from the group consisting of T52I, I56V, G60V, D61G, D61V, D61Y, Y⁶²D, Y⁶³D, Y⁶³C, E69K, E69Q, K70R, A⁷²S, A⁷²T, A⁷²V, T731, E76A, E76G, E76K, E76Q, E123D, E139D, S189A, T253M, Q257L, L261F, L261H, L262R, R²⁶⁵Q, F285S, N308D, V428M, A⁴⁶¹T, A⁴⁶¹G, T468M, P491S, S502L, S502P, G503A, G503V, M504V, Q506P, T507K, Q510P, Q510H, and a combination thereof.

In some embodiments, the SHP2 mutation is at a position selected from the group consisting of G60, D61, A⁷², E76, G503 and S502, and a combination thereof. In some embodiments, the SHP2 mutation is at a position selected from the group consisting of G60, D61, E69, A⁷², E123, Y¹⁹⁷, N308, V428, A⁴⁶¹, T468, S502, G503, T507, and a combination thereof. In some embodiments, the SHP2 mutation is selected from the group consisting of G60V, D61G, D61V, D61Y, E69K, E69Q, A⁷²S, A⁷²T, A⁷²V, E123D, N308D, V428M, A⁴⁶¹T, A⁴⁶¹G, T468M, S502L, S502P, G503A, G503V, T507K, and a combination thereof. In some embodiments, the SHP2 mutation is at a position selected from the group consisting of T52, 156, Y⁶², Y⁶³, E69, K70, E139, L261, R²⁶⁵, N308, T468, M504, Q510, and a combination thereof. In some embodiments, the SHP2 mutation is selected from the group consisting of T52I, I56V, Y⁶²D, Y⁶³D, Y⁶³C, E69K, E69Q, K70R, E139D, L261F, L261H, R²⁶⁵Q, N308D, T468M, M504V, Q510P, Q510H, and a combination thereof.

In some embodiments, the SHP2 mutation is expressed in a subject after a course of treatment with a SHP2 inhibitor. In some embodiments, the SHP2 mutation is expressed in a subject after a course of treatment with an allosteric SHP2 inhibitor. In some embodiments, the SHP2 mutation is expressed in a subject after a course of treatment with an active site SHP2 inhibitor. In some embodiments, the SHP2 mutation is expressed in a subject after a course of treatment with an allosteric SHP2 inhibitor. The SHP2 inhibitor, e.g., allosteric SHP2 inhibitor, is generally an inhibitor of wild-type SHP2 protein. SHP2 inhibitors, e.g., allosteric SHP2 inhibitors, may be selected from among those disclosed, without limitation, in WO 2021149817, WO 2021148010, WO 2021147879, WO 2021143823, WO 2021143701, WO 2021143680, WO2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US20210085677, U.S. Pat. Nos. 10,858,359, 10,934,302 and 10,954,243, or a pharmaceutically acceptable salt, solvate, isomer (e.g., stereoisomer), prodrug, or tautomer thereof. A non-limiting list of exemplary such allosteric SHP2 inhibitors include ERAS-601, BBP-398, RLY-1971, JAB-3068, JAB-3312, TNO155, SHP099, RMC-4550, and RMC-4630, or a pharmaceutically acceptable salt, solvate, isomer (e.g., stereoisomer), prodrug, or tautomer thereof. In some embodiments, the SHP2 inhibitor is TNO155, or a pharmaceutically acceptable salt, solvate, isomer (e.g., stereoisomer), prodrug, or tautomer thereof. In some embodiments, the SHP2 inhibitor is RMC-4630, or a pharmaceutically acceptable salt, solvate, isomer (e.g., stereoisomer), prodrug, or tautomer thereof. A non-limiting list of exemplary active-site SHP2 inhibitors include NSC-87877, IIB-08, 11a-1, and GS-493, or a pharmaceutically acceptable salt, solvate, isomer (e.g., stereoisomer), prodrug, or tautomer thereof. In some embodiments, a course of treatment with these SHP2 inhibitors, including allosteric SHP2 inhibitors, active site SHP2 inhibitors, or other such inhibitors of wild type SHP2, may induce a mutation, e.g., an activating mutation, in SHP2. In some embodiments, the SHP2 mutation confers resistance to the SHP2 inhibitor. Alternatively, the resistance to the SHP2 inhibitor may occur due to a natural mutation in the SHP2 protein. In any event, the mutation may induce pathway reactivation and drug resistance to the SHP2 inhibitor.

Accordingly, the present invention is directed to a method of treating a subject having a disease or disorder associated with cells having a mutation in SHP2 protein. In some embodiments, the mutation is an activating SHP2 mutation. According to embodiments of the present invention, the method comprises administering to the subject a therapeutically effective amount of a SOS1 inhibitor. In some embodiments, SOS1 inhibitors may also inhibit SOS2, i.e., the method comprises administering to the subject a therapeutically effective amount of a dual SOS1/SOS2 inhibitor. In some embodiments, such a SOS1 inhibitor is characterized by a selectivity ratio less than 10-fold for inhibition of SOS1 relative to SOS2. In some embodiments, the method comprises administering to the subject a therapeutically effective amount of a selective SOS1 inhibitor. In some embodiments, such a SOS1 inhibitor is characterized by a selectivity ratio greater of at least about 10-fold, such as greater than at least about 30-fold, for inhibition of SOS1 relative to SOS2.

### SOS1 Inhibitors

In some embodiments, the SOS1 inhibitor is selected from those disclosed in WO 2018/115380, WO 2018/172250, WO 2019/122129, and WO 2019/201848, or a pharmaceutically acceptable salt, solvate, isomer (e.g., stereoisomer), prodrug, or tautomer thereof, the disclosures of each of which are hereby incorporated by reference as if set forth in their entirety. In some embodiments, the SOS1 inhibitor is selected from those disclosed in WO 2021/092115, WO 2020/180768, and WO 2020/180770, or a pharmaceutically acceptable salt, solvate, isomer (e.g., stereoisomer), prodrug, or tautomer thereof, the disclosures of each of which are hereby incorporated by reference as if set forth in their entirety.

In some embodiments, the SOS1 inhibitor is a compound having the structure of Formula (41-I),

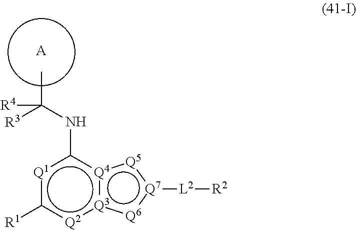

or a pharmaceutically acceptable salt, solvate, isomer (e.g., stereoisomer), prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a} or —OR^{1a}, wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

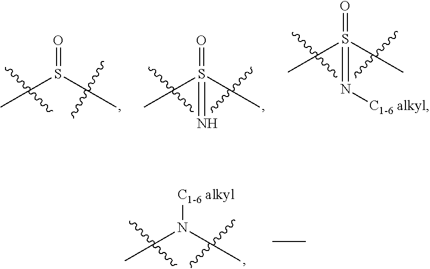

C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}—C(O)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂) OCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

In some embodiments, the SOS1 inhibitor is a compound having the structure of Formula (41-I-a),

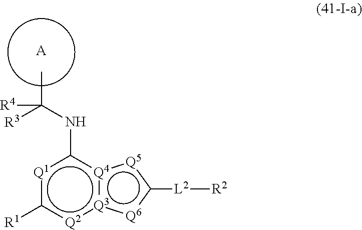

or a pharmaceutically acceptable salt, solvate, isomer (e.g., stereoisomer), prodrug, or tautomer thereof, wherein:
Q¹, Q², Q⁵ and A are as defined in Formula (41-I);
Q³ and Q⁴ are independently C or N, wherein at least one of Q³ and Q⁴ is C;
Q⁶ is CH, N, NH, O, or S;
wherein at least one of Q¹, Q², Q³, Q⁴, Q⁵, and Q⁶ is N, NH, O, or S;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O) (CH₂)ₚ—, —(CH₂)ₚ—, and —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂) OCH₃, wherein r is 1, 2, or 3; wherein R^{2b} is H or C₁₋₆ alkyl; and wherein R^{2c} is H or C₁₋₆ alkyl; and
R³ and R⁴ are independently H or C₁₋₆ alkyl; wherein at least one of R³ and R⁴ is not H; or R³ and R⁴ together with the atom to which they are attached combine to form a 3-6 membered cycloalkyl.

In some embodiments, the SOS1 inhibitor is a compound having the structure of Formula (41-II),

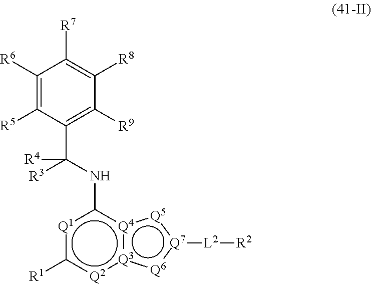

or a pharmaceutically acceptable salt, solvate, isomer (e.g., stereoisomer), prodrug, or tautomer thereof, wherein:
L², Q¹, Q², Q³, Q¹, Q⁵, Q⁶, Q⁷, R¹, R², R³ and R⁴ are as defined in Formula (41-I);
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with-OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound having the structure of Formula (41-III),

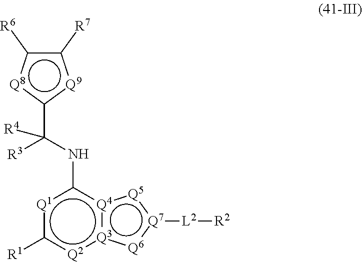

or a pharmaceutically acceptable salt, solvate, isomer (e.g., stereoisomer), prodrug, or tautomer thereof, wherein:
L², Q¹, Q², Q³, Q⁴, Q⁵, Q⁶, Q⁷, R¹, R², R³ and R⁴ are as defined in Formula (41-1);
Q⁸ and Q⁹ are independently CH, N, NH, O, or S, provided at least one of Q⁸ and Q⁹ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with-OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound selected from the group consisting of the compounds in the following table, or a pharmaceutically acceptable salt, solvate, isomer (e.g., stereoisomer), prodrug, or tautomer thereof:

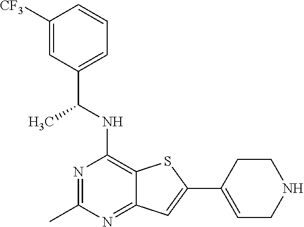 [SMILES: Cc1nc(N[C@H](C)c2cccc(C(F)(F)F)c2)c2sc(C3=CCNCC3)cc2n1]

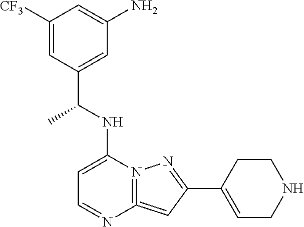

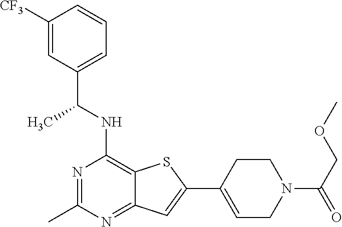 [SMILES: COCC(=O)N1CC=C(c2cc3nc(C)nc(N[C@H](C)c4cccc(C(F)(F)F)c4)c3s2)CC1]

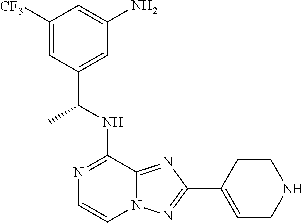 [SMILES: C[C@@H](Nc1nccn2nc(C3=CCNCC3)nc12)c1cc(N)cc(C(F)(F)F)c1]

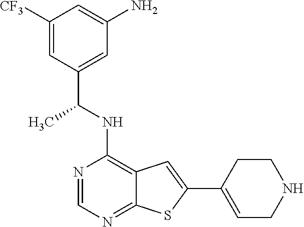 [SMILES: C[C@@H](Nc1ncnc2sc(C3=CCNCC3)cc12)c1cc(N)cc(C(F)(F)F)c1]

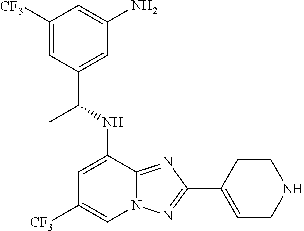 [SMILES: C[C@@H](Nc1cc(C(F)(F)F)cn2nc(C3=CCNCC3)nc12)c1cc(N)cc(C(F)(F)F)c1]

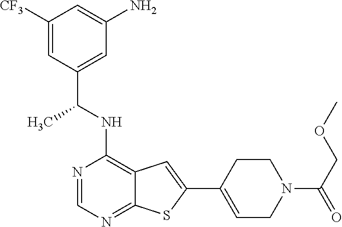 [SMILES: COCC(=O)N1CC=C(c2cc3c(N[C@H](C)c4cc(N)cc(C(F)(F)F)c4)ncnc3s2)CC1]

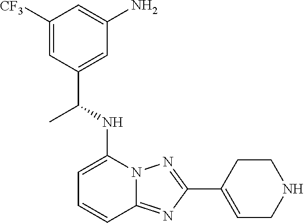 [SMILES: C[C@@H](Nc1cccc2nc(C3=CCNCC3)nn12)c1cc(N)cc(C(F)(F)F)c1]

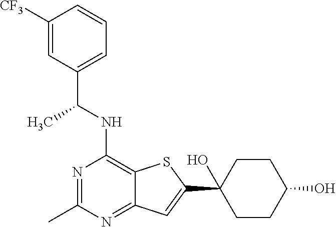 [SMILES: Cc1nc(N[C@H](C)c2cccc(C(F)(F)F)c2)c2sc([C@]3(O)CC[C@@H](O)CC3)cc2n1]

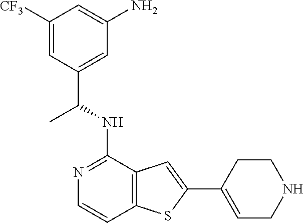

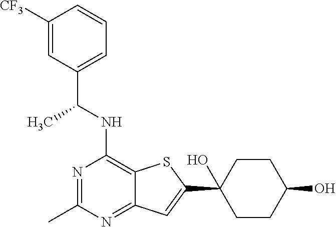 [SMILES: Cc1nc(N[C@H](C)c2cccc(C(F)(F)F)c2)c2sc([C@]3(O)CC[C@H](O)CC3)cc2n1]

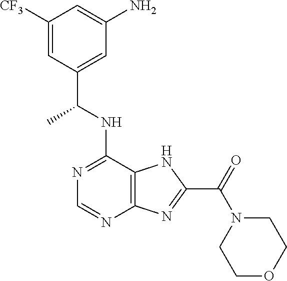 [SMILES: C[C@@H](Nc1ncnc2nc(C(=O)N3CCOCC3)[nH]c12)c1cc(N)cc(C(F)(F)F)c1]

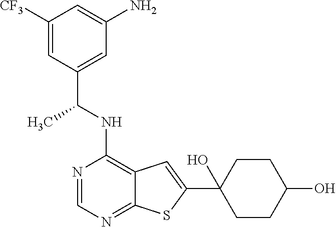 [SMILES: C[C@@H](Nc1ncnc2sc(C3(O)CCC(O)CC3)cc12)c1cc(N)cc(C(F)(F)F)c1]

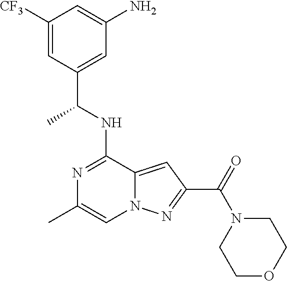 [SMILES: Cc1cn2nc(C(=O)N3CCOCC3)cc2c(N[C@H](C)c2cc(N)cc(C(F)(F)F)c2)n1]

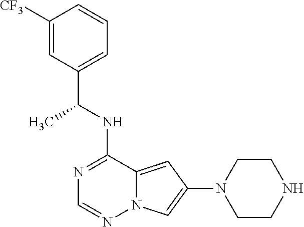 [SMILES: C[C@@H](Nc1ncnn2cc(N3CCNCC3)cc12)c1cccc(C(F)(F)F)c1]

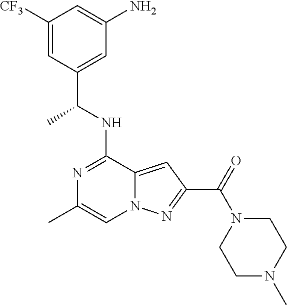 [SMILES: Cc1cn2nc(C(=O)N3CCN(C)CC3)cc2c(N[C@H](C)c2cc(N)cc(C(F)(F)F)c2)n1]

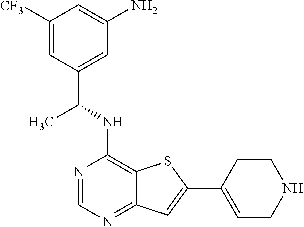 [SMILES: C[C@@H](Nc1ncnc2cc(C3=CCNCC3)sc12)c1cc(N)cc(C(F)(F)F)c1]

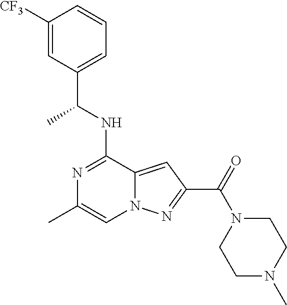 [SMILES: Cc1cn2nc(C(=O)N3CCN(C)CC3)cc2c(N[C@H](C)c2cccc(C(F)(F)F)c2)n1]

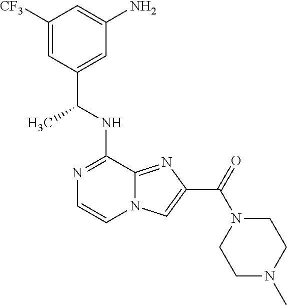 [SMILES: C[C@@H](Nc1nccn2cc(C(=O)N3CCN(C)CC3)nc12)c1cc(N)cc(C(F)(F)F)c1]

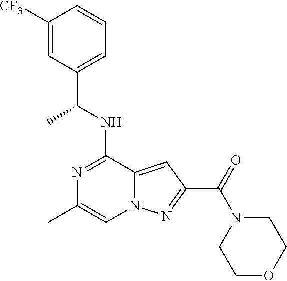 [SMILES: Cc1cn2nc(C(=O)N3CCOCC3)cc2c(N[C@H](C)c2cccc(C(F)(F)F)c2)n1]

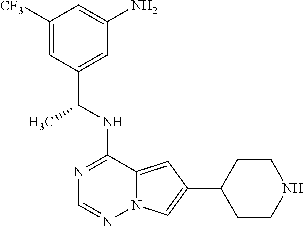 [SMILES: C[C@@H](Nc1ncnn2cc(C3CCNCC3)cc12)c1cc(N)cc(C(F)(F)F)c1]

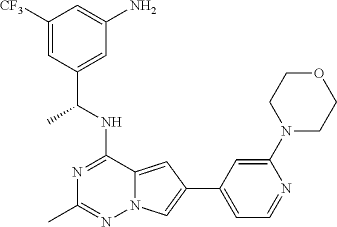 [SMILES: Cc1nc(N[C@H](C)c2cc(N)cc(C(F)(F)F)c2)c2cc(-c3ccnc(N4CCOCC4)c3)cn2n1]

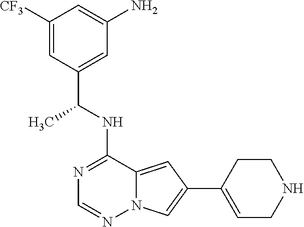 [SMILES: C[C@@H](Nc1ncnn2cc(C3=CCNCC3)cc12)c1cc(N)cc(C(F)(F)F)c1]

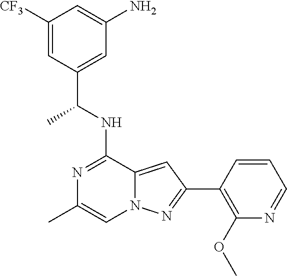 [SMILES: COc1ncccc1-c1cc2c(N[C@H](C)c3cc(N)cc(C(F)(F)F)c3)nc(C)cn2n1]

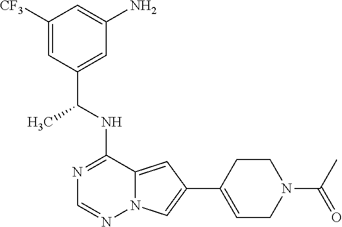 [SMILES: CC(=O)N1CC=C(c2cc3c(N[C@H](C)c4cc(N)cc(C(F)(F)F)c4)ncnn3c2)CC1]

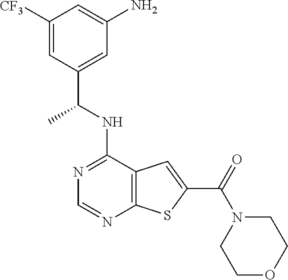 [SMILES: C[C@@H](Nc1ncnc2sc(C(=O)N3CCOCC3)cc12)c1cc(N)cc(C(F)(F)F)c1]

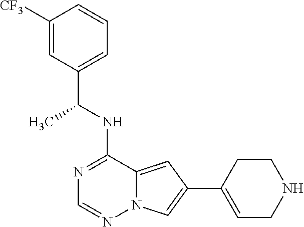 [SMILES: C[C@@H](Nc1ncnn2cc(C3=CCNCC3)cc12)c1cccc(C(F)(F)F)c1]

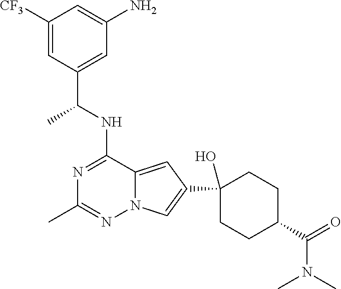 [SMILES: Cc1nc(N[C@H](C)c2cc(N)cc(C(F)(F)F)c2)c2cc([C@]3(O)CC[C@H](C(=O)N(C)C)CC3)cn2n1]

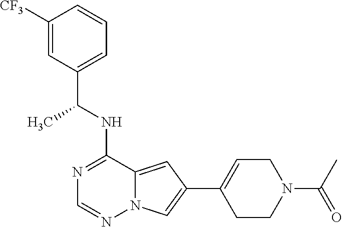 [SMILES: CC(=O)N1CC=C(c2cc3c(N[C@H](C)c4cccc(C(F)(F)F)c4)ncnn3c2)CC1]

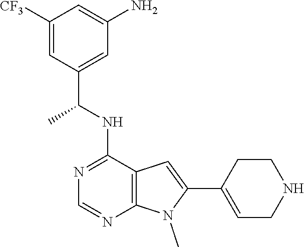 [SMILES: C[C@@H](Nc1ncnc2c1cc(C1=CCNCC1)n2C)c1cc(N)cc(C(F)(F)F)c1]

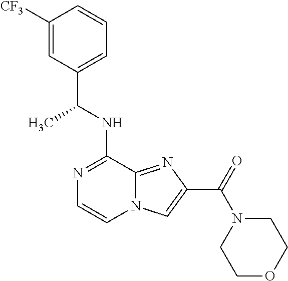 [SMILES: C[C@@H](Nc1nccn2cc(C(=O)N3CCOCC3)nc12)c1cccc(C(F)(F)F)c1]

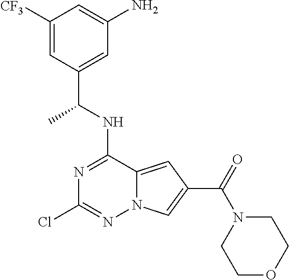 [SMILES: C[C@@H](Nc1nc(Cl)nn2cc(C(=O)N3CCOCC3)cc12)c1cc(N)cc(C(F)(F)F)c1]

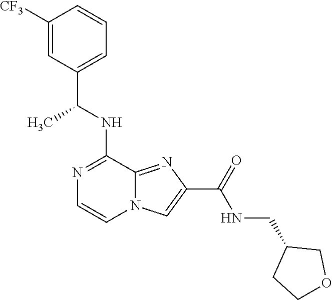 [SMILES: C[C@@H](Nc1nccn2cc(C(=O)NC[C@H]3CCOC3)nc12)c1cccc(C(F)(F)F)c1]

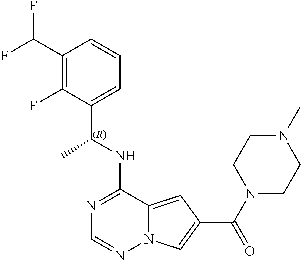

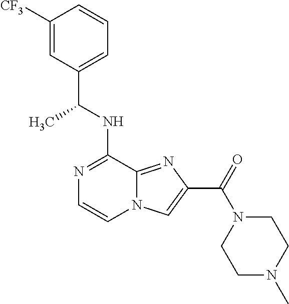 [SMILES: C[C@@H](Nc1nccn2cc(C(=O)N3CCN(C)CC3)nc12)c1cccc(C(F)(F)F)c1]

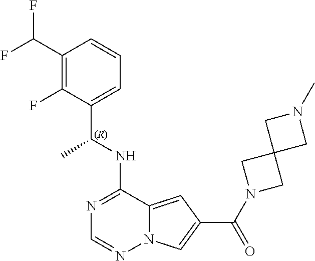

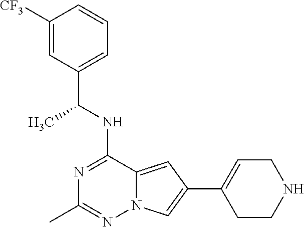 [SMILES: Cc1nc(N[C@H](C)c2cccc(C(F)(F)F)c2)c2cc(C3=CCNCC3)cn2n1]

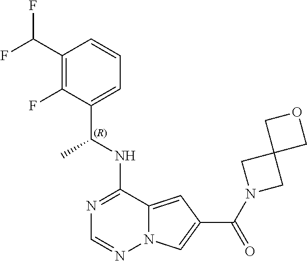

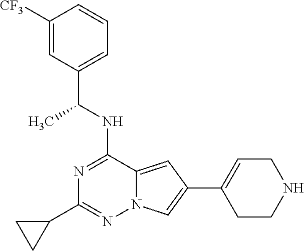 [SMILES: C[C@@H](Nc1nc(C2CC2)nn2cc(C3=CCNCC3)cc12)c1cccc(C(F)(F)F)c1]

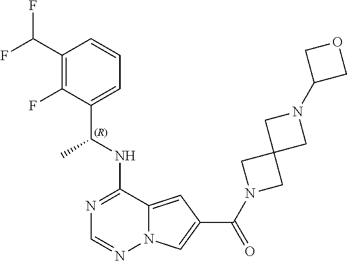

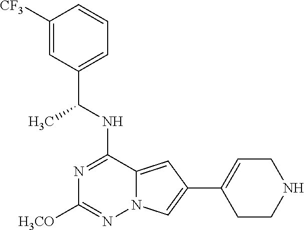 [SMILES: COc1nc(N[C@H](C)c2cccc(C(F)(F)F)c2)c2cc(C3=CCNCC3)cn2n1]

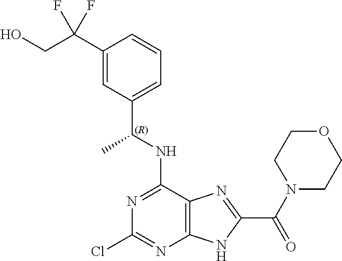

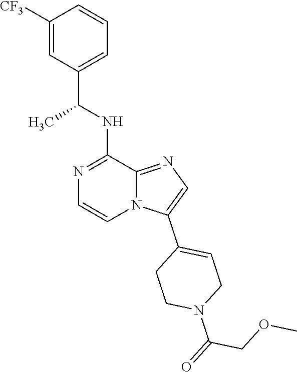 [SMILES: COCC(=O)N1CC=C(c2cnc3c(N[C@H](C)c4cccc(C(F)(F)F)c4)nccn23)CC1]

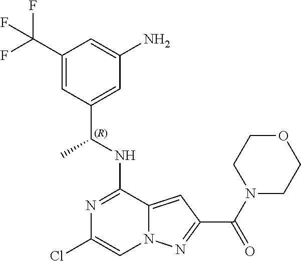

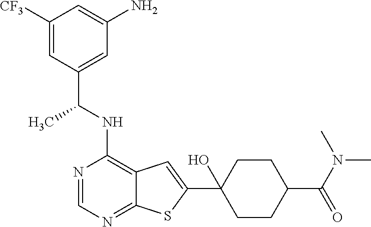 [SMILES: C[C@@H](Nc1ncnc2sc(C3(O)CCC(C(=O)N(C)C)CC3)cc12)c1cc(N)cc(C(F)(F)F)c1]

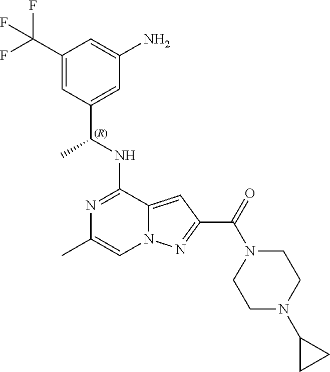

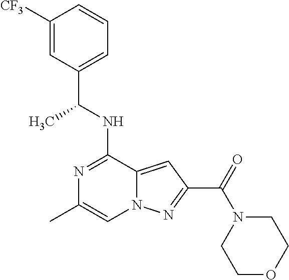 [SMILES: Cc1cn2nc(C(=O)N3CCOCC3)cc2c(N[C@H](C)c2cccc(C(F)(F)F)c2)n1]

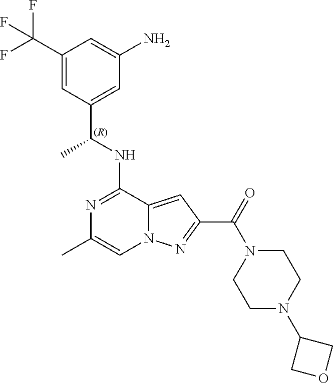

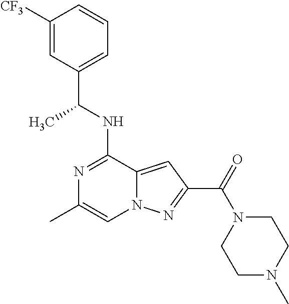 [SMILES: Cc1cn2nc(C(=O)N3CCN(C)CC3)cc2c(N[C@H](C)c2cccc(C(F)(F)F)c2)n1]

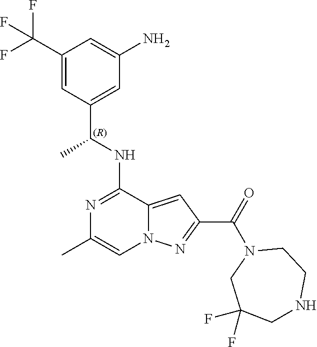

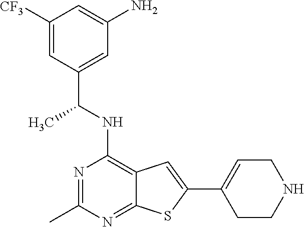 [SMILES: Cc1nc(N[C@H](C)c2cc(N)cc(C(F)(F)F)c2)c2cc(C3=CCNCC3)sc2n1]

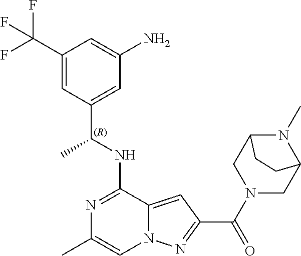

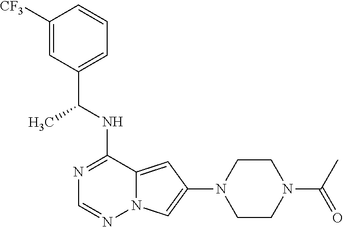 [SMILES: CC(=O)N1CCN(c2cc3c(N[C@H](C)c4cccc(C(F)(F)F)c4)ncnn3c2)CC1]

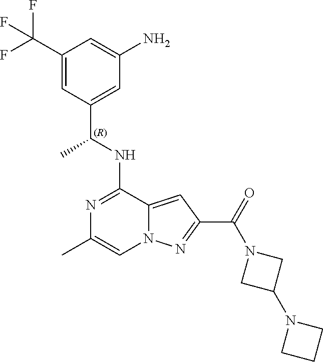

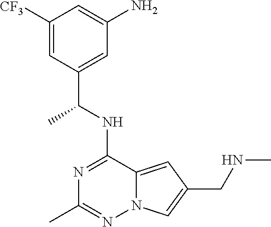 [SMILES: CNCc1cc2c(N[C@H](C)c3cc(N)cc(C(F)(F)F)c3)nc(C)nn2c1]

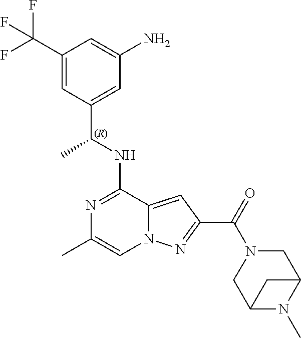

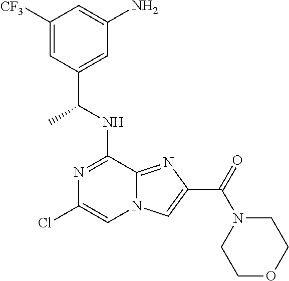 [SMILES: C[C@@H](Nc1nc(Cl)cn2cc(C(=O)N3CCOCC3)nc12)c1cc(N)cc(C(F)(F)F)c1]

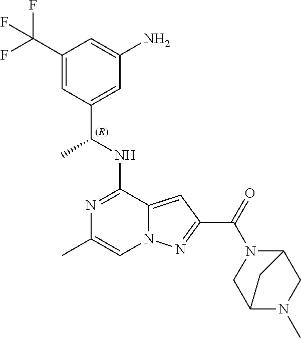

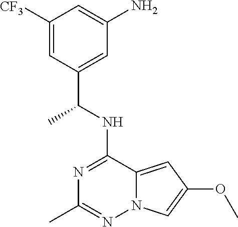 [SMILES: COc1cc2c(N[C@H](C)c3cc(N)cc(C(F)(F)F)c3)nc(C)nn2c1]

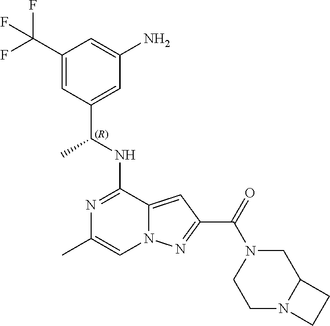

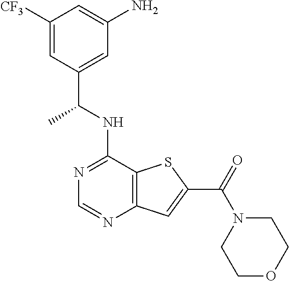 [SMILES: C[C@@H](Nc1ncnc2cc(C(=O)N3CCOCC3)sc12)c1cc(N)cc(C(F)(F)F)c1]

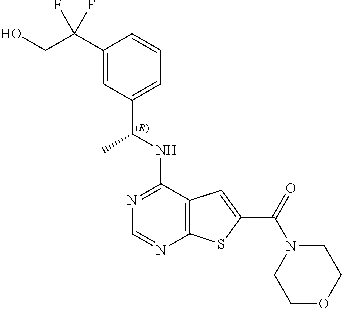

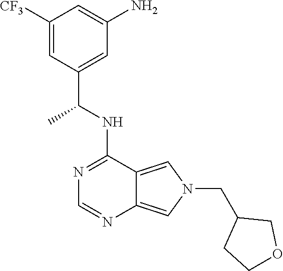 [SMILES: C[C@@H](Nc1ncnc2cn(CC3CCOC3)cc12)c1cc(N)cc(C(F)(F)F)c1]

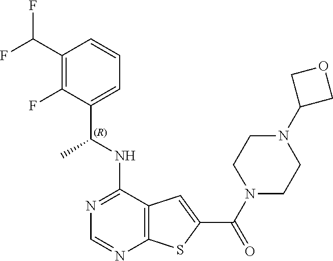

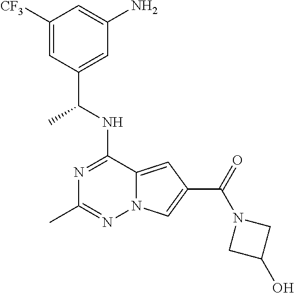 [SMILES: Cc1nc(N[C@H](C)c2cc(N)cc(C(F)(F)F)c2)c2cc(C(=O)N3CC(O)C3)cn2n1]

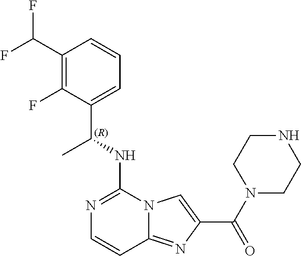

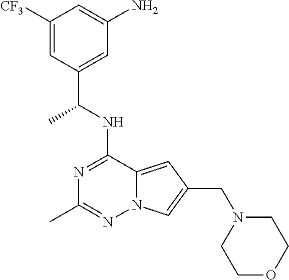 [SMILES: Cc1nc(N[C@H](C)c2cc(N)cc(C(F)(F)F)c2)c2cc(CN3CCOCC3)cn2n1]

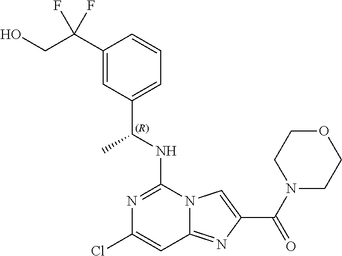

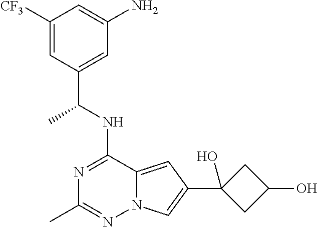 [SMILES: Cc1nc(N[C@H](C)c2cc(N)cc(C(F)(F)F)c2)c2cc(C3(O)CC(O)C3)cn2n1]

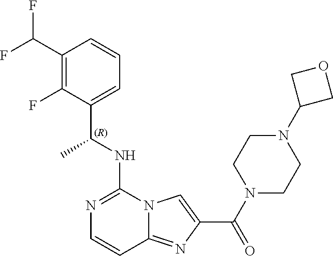

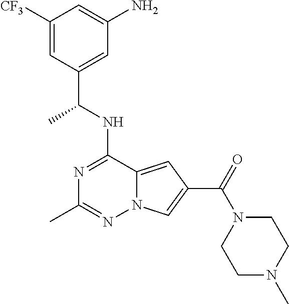 [SMILES: Cc1nc(N[C@H](C)c2cc(N)cc(C(F)(F)F)c2)c2cc(C(=O)N3CCN(C)CC3)cn2n1]

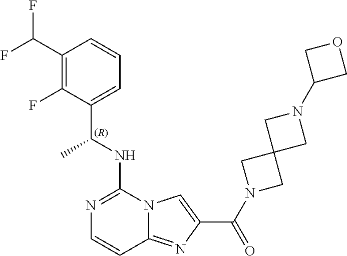

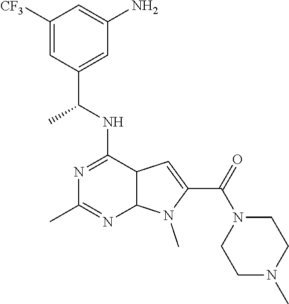 [SMILES: CC1=NC2C(C=C(C(=O)N3CCN(C)CC3)N2C)C(N[C@H](C)c2cc(N)cc(C(F)(F)F)c2)=N1]

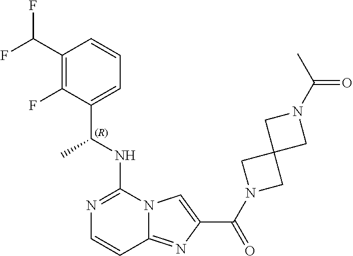

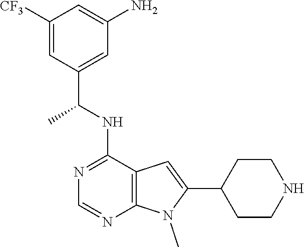 [SMILES: C[C@@H](Nc1ncnc2c1cc(C1CCNCC1)n2C)c1cc(N)cc(C(F)(F)F)c1]

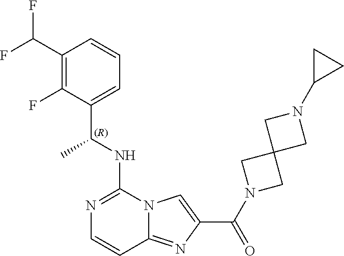

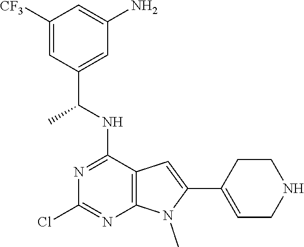 [SMILES: C[C@@H](Nc1nc(Cl)nc2c1cc(C1=CCNCC1)n2C)c1cc(N)cc(C(F)(F)F)c1]

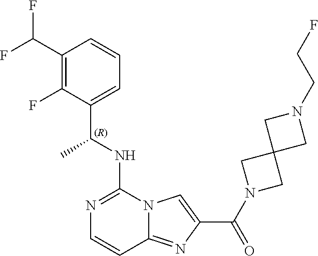

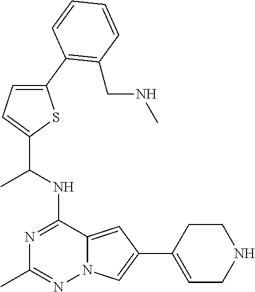 [SMILES: CNCc1ccccc1-c1ccc(C(C)Nc2nc(C)nn3cc(C4=CCNCC4)cc23)s1]

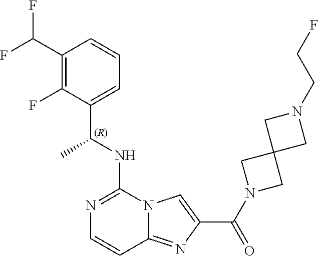

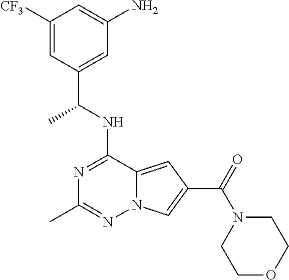 [SMILES: Cc1nc(N[C@H](C)c2cc(N)cc(C(F)(F)F)c2)c2cc(C(=O)N3CCOCC3)cn2n1]

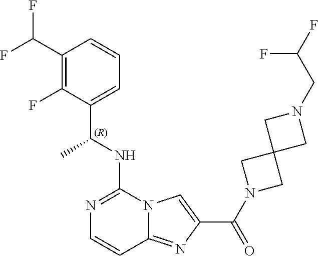

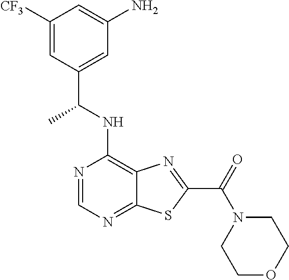 [SMILES: C[C@@H](Nc1ncnc2sc(C(=O)N3CCOCC3)nc12)c1cc(N)cc(C(F)(F)F)c1]

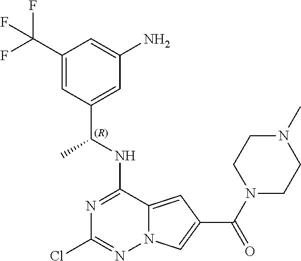

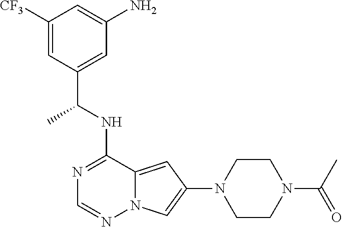 [SMILES: CC(=O)N1CCN(c2cc3c(N[C@H](C)c4cc(N)cc(C(F)(F)F)c4)ncnn3c2)CC1]

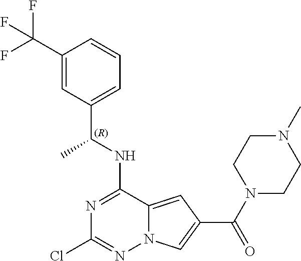

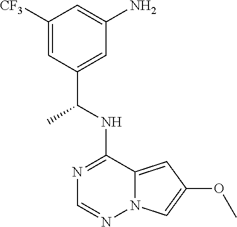 [SMILES: COc1cc2c(N[C@H](C)c3cc(N)cc(C(F)(F)F)c3)ncnn2c1]

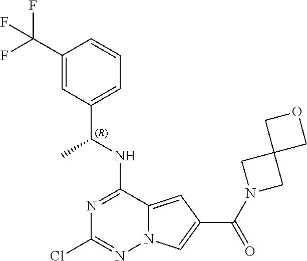

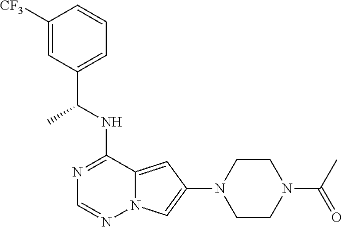 [SMILES: CC(=O)N1CCN(c2cc3c(N[C@H](C)c4cccc(C(F)(F)F)c4)ncnn3c2)CC1]

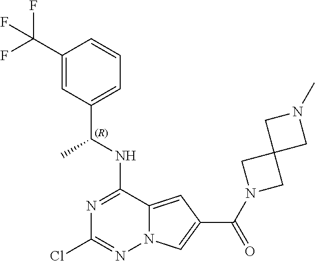

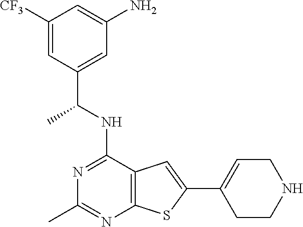 [SMILES: Cc1nc(N[C@H](C)c2cc(N)cc(C(F)(F)F)c2)c2cc(C3=CCNCC3)sc2n1]

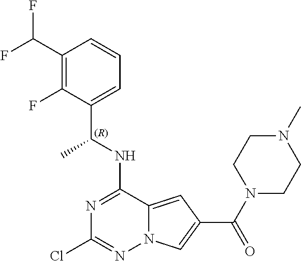

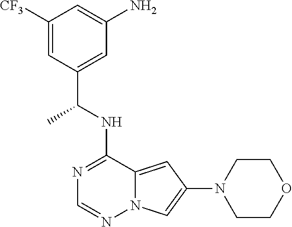 [SMILES: C[C@@H](Nc1ncnn2cc(N3CCOCC3)cc12)c1cc(N)cc(C(F)(F)F)c1]

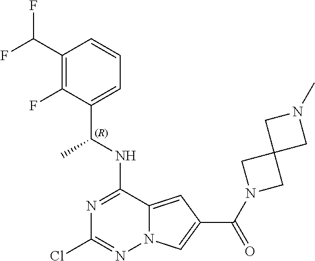

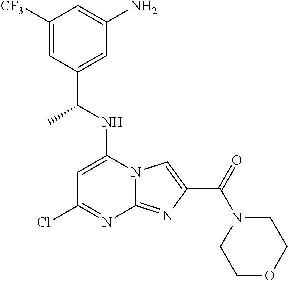 [SMILES: C[C@@H](Nc1cc(Cl)nc2nc(C(=O)N3CCOCC3)cn12)c1cc(N)cc(C(F)(F)F)c1]

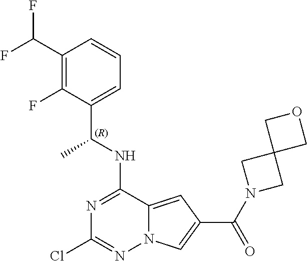

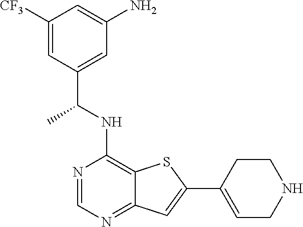 [SMILES: C[C@@H](Nc1ncnc2cc(C3=CCNCC3)sc12)c1cc(N)cc(C(F)(F)F)c1]

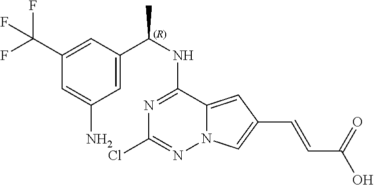

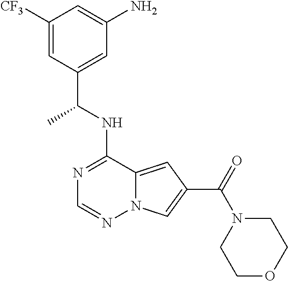 [SMILES: C[C@@H](Nc1ncnn2cc(C(=O)N3CCOCC3)cc12)c1cc(N)cc(C(F)(F)F)c1]

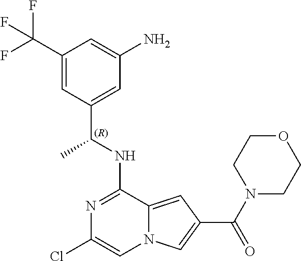

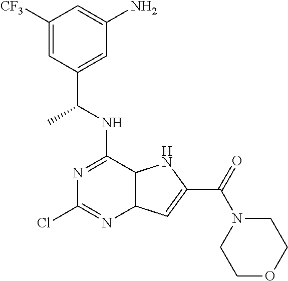 [SMILES: C[C@@H](NC1=NC(Cl)=NC2C=C(C(=O)N3CCOCC3)NC12)c1cc(N)cc(C(F)(F)F)c1]

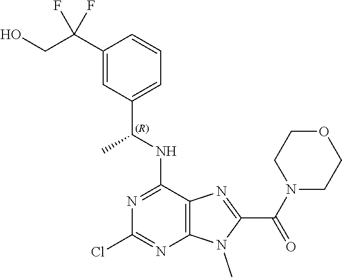

In some embodiments, the SOS1 inhibitor is a compound having the structure of Formula (42-I),

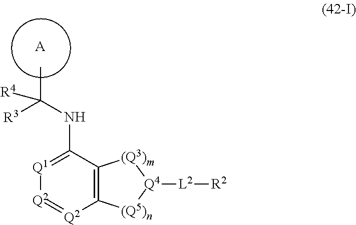

or a pharmaceutically acceptable salt, solvate, isomer (e.g., stereoisomer), prodrug, or tautomer thereof, wherein:
Q¹ is CH or N;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each RON is independently H, C₁₋₆ alkyl, or 6-10 membered aryl;
wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}, wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

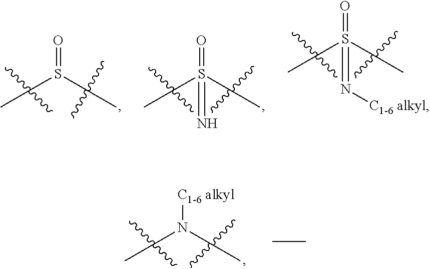

—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH;
wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl;
with the proviso that when

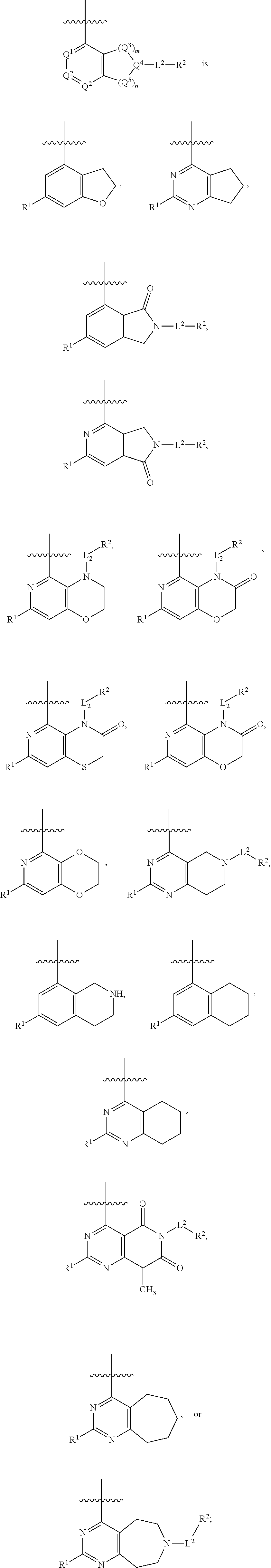

then R¹ is not H.

In some embodiments, the SOS1 inhibitor is a compound having the structure of Formula (42-I-a),

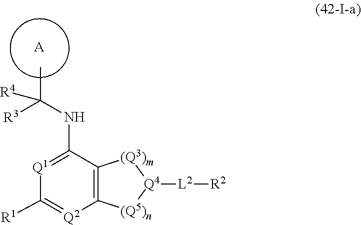

or a pharmaceutically acceptable salt, solvate, isomer (e.g., stereoisomer), prodrug, or tautomer thereof, wherein:
Q¹, Q³, Q⁴, Q⁵, m, n and A are as defined in Formula (42-I);
Q² is CH or N;
wherein at least one of Q¹, Q², Q³, Q⁴, and Q⁵ is N, NR^{QN}, O, or SO₂;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O) (CH₂)ₚ—, —(CH₂)ₚ—, or —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂),OCH₃, wherein r is 1, 2, or 3; wherein R^{2b} is H or C₁₋₆ alkyl; and wherein R^{2c} is H or C₁₋₆ alkyl; and
R³ and R⁴ are independently H or C₁₋₆ alkyl; wherein at least one of R³ and R⁴ is not H; or R³ and R⁴ together with the atom to which they are attached combine to form a 3-6 membered cycloalkyl.

In some embodiments, the SOS1 inhibitor is a compound having the structure of Formula (42-V),

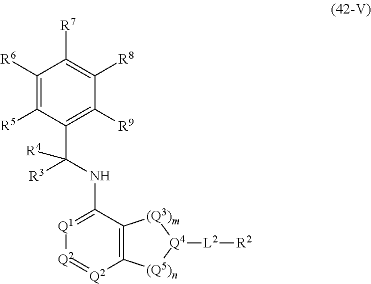

or a pharmaceutically acceptable salt, solvate, isomer (e.g., stereoisomer), prodrug, or tautomer thereof, wherein:
L², Q¹, Q², Q³, Q⁴, Q⁵, m, n, R¹, R², R³ and R⁴ are as defined in Formula (42-I);
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, halogen, —NO₂, oxo, —CN, —R¹⁰, —OR¹⁰, —NR¹¹R¹², —SR¹⁰, —S(O),NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ forms a 3-14 membered fused ring;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound having the structure of Formula (42-VI),

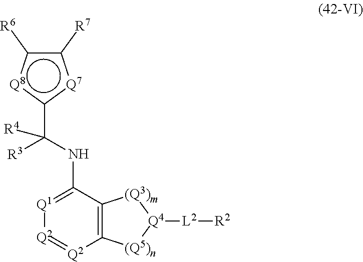

or a pharmaceutically acceptable salt, solvate, isomer (e.g., stereoisomer), prodrug, or tautomer thereof, wherein:
L², Q¹, Q², Q³, Q⁴, Q⁵, m, n, R¹, R², R³, and R⁴ are as defined in Formula (42-I);
Q⁷ and Q⁸ are each independently CH, N, NH, O, or S, provided at least one of Q⁷ and Q⁸ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S (O) NR¹¹R¹², —NR¹⁰S(O)R¹¹, 3-8 membered cycloalkyl, 3-14 membered heterocyclyl, 6-10 membered aryl, or 5-10 membered heteroaryl,
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound selected from the group consisting of the compounds in the following table, or a pharmaceutically acceptable salt, solvate, isomer (e.g., stereoisomer), prodrug, or tautomer thereof:

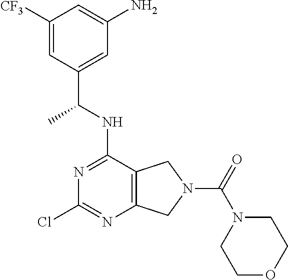 [SMILES: C[C@@H](Nc1nc(Cl)nc2c1CN(C(=O)N1CCOCC1)C2)c1cc(N)cc(C(F)(F)F)c1]

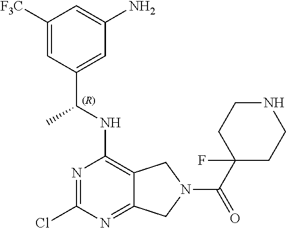

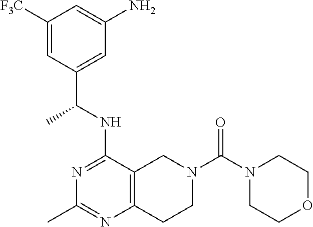 [SMILES: Cc1nc2c(c(N[C@H](C)c3cc(N)cc(C(F)(F)F)c3)n1)CN(C(=O)N1CCOCC1)CC2]

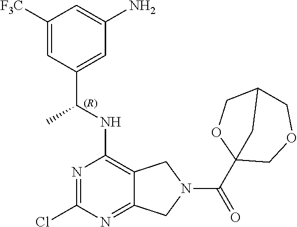

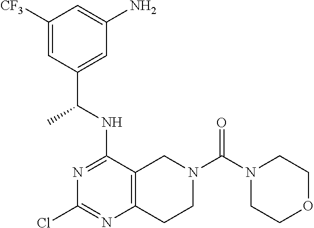 [SMILES: C[C@@H](Nc1nc(Cl)nc2c1CN(C(=O)N1CCOCC1)CC2)c1cc(N)cc(C(F)(F)F)c1]

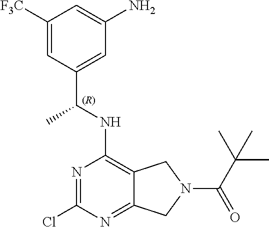

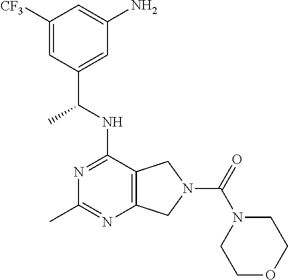 [SMILES: Cc1nc2c(c(N[C@H](C)c3cc(N)cc(C(F)(F)F)c3)n1)CN(C(=O)N1CCOCC1)C2]

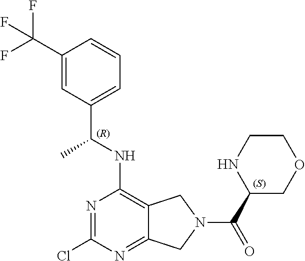

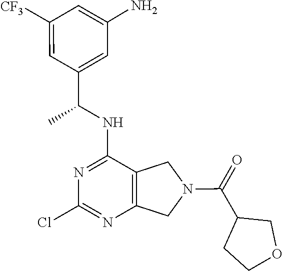 [SMILES: C[C@@H](Nc1nc(Cl)nc2c1CN(C(=O)C1CCOC1)C2)c1cc(N)cc(C(F)(F)F)c1]

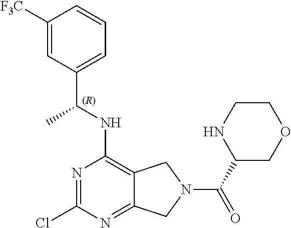

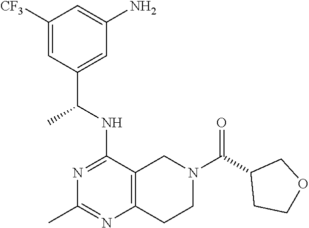

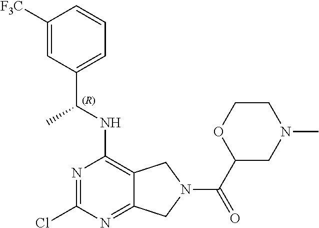

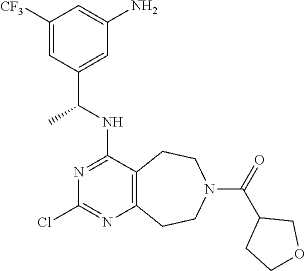 [SMILES: C[C@@H](Nc1nc(Cl)nc2c1CCN(C(=O)C1CCOC1)CC2)c1cc(N)cc(C(F)(F)F)c1]

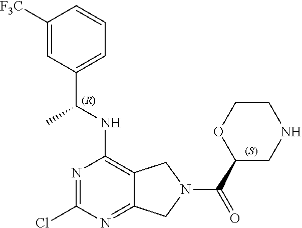

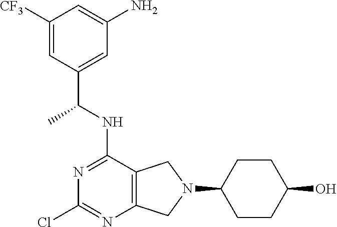 [SMILES: C[C@@H](Nc1nc(Cl)nc2c1CN([C@H]1CC[C@@H](O)CC1)C2)c1cc(N)cc(C(F)(F)F)c1]

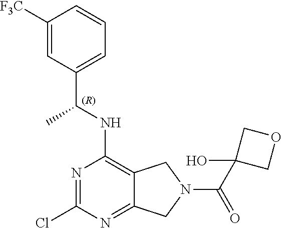

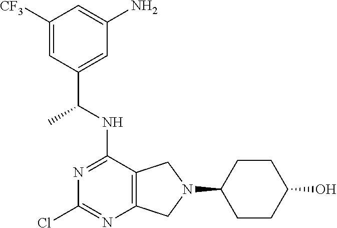 [SMILES: C[C@@H](Nc1nc(Cl)nc2c1CN([C@H]1CC[C@H](O)CC1)C2)c1cc(N)cc(C(F)(F)F)c1]

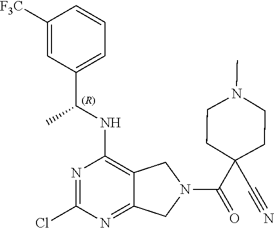

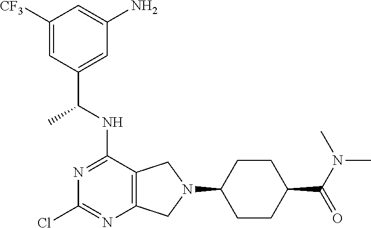 [SMILES: C[C@@H](Nc1nc(Cl)nc2c1CN([C@H]1CC[C@@H](C(=O)N(C)C)CC1)C2)c1cc(N)cc(C(F)(F)F)c1]

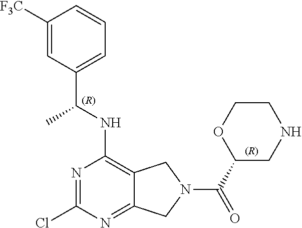

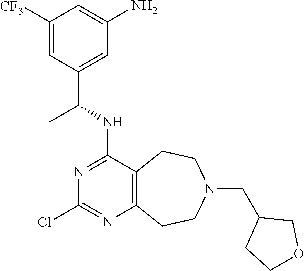 [SMILES: C[C@@H](Nc1nc(Cl)nc2c1CCN(CC1CCOC1)CC2)c1cc(N)cc(C(F)(F)F)c1]

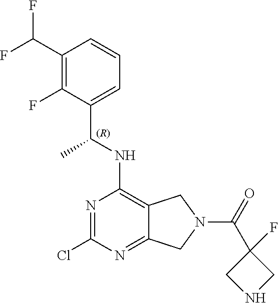

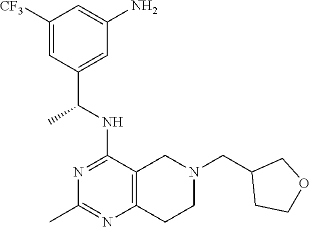 [SMILES: Cc1nc2c(c(N[C@H](C)c3cc(N)cc(C(F)(F)F)c3)n1)CN(CC1CCOC1)CC2]

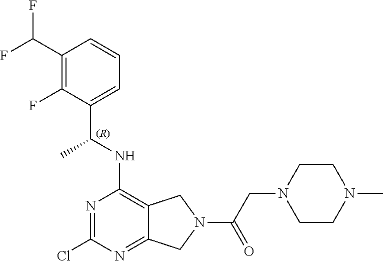

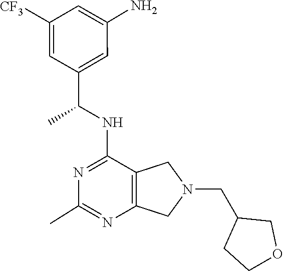 [SMILES: Cc1nc2c(c(N[C@H](C)c3cc(N)cc(C(F)(F)F)c3)n1)CN(CC1CCOC1)C2]

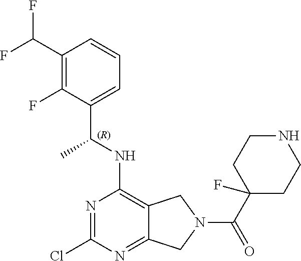

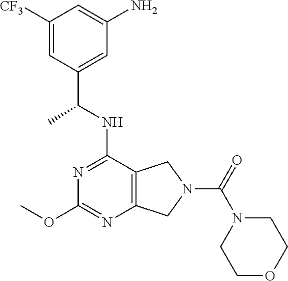 [SMILES: COc1nc2c(c(N[C@H](C)c3cc(N)cc(C(F)(F)F)c3)n1)CN(C(=O)N1CCOCC1)C2]

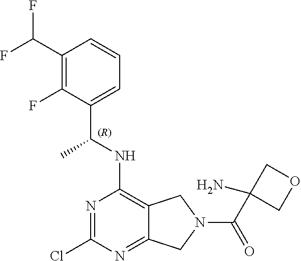

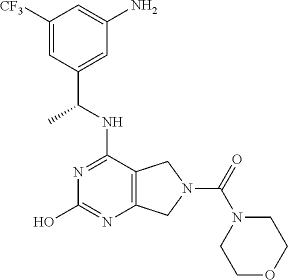 [SMILES: C[C@@H](Nc1nc(O)nc2c1CN(C(=O)N1CCOCC1)C2)c1cc(N)cc(C(F)(F)F)c1]

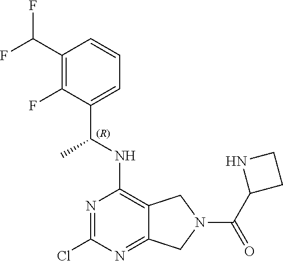

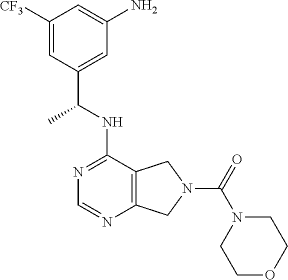 [SMILES: C[C@@H](Nc1ncnc2c1CN(C(=O)N1CCOCC1)C2)c1cc(N)cc(C(F)(F)F)c1]

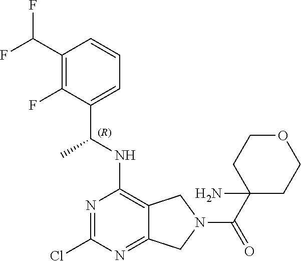

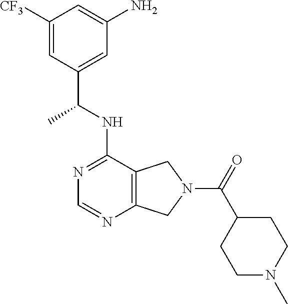 [SMILES: C[C@@H](Nc1ncnc2c1CN(C(=O)C1CCN(C)CC1)C2)c1cc(N)cc(C(F)(F)F)c1]

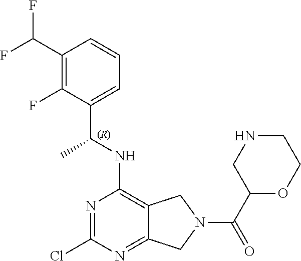

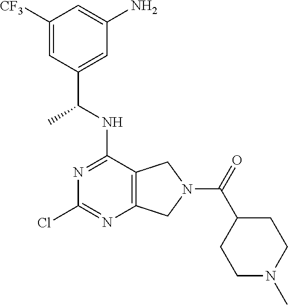 [SMILES: C[C@@H](Nc1nc(Cl)nc2c1CN(C(=O)C1CCN(C)CC1)C2)c1cc(N)cc(C(F)(F)F)c1]

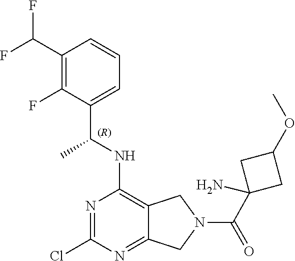

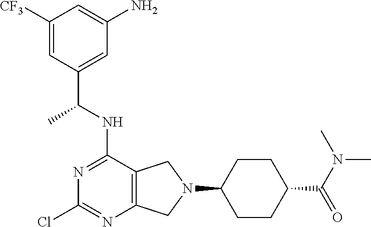 [SMILES: C[C@@H](Nc1nc(Cl)nc2c1CN([C@H]1CC[C@H](C(=O)N(C)C)CC1)C2)c1cc(N)cc(C(F)(F)F)c1]

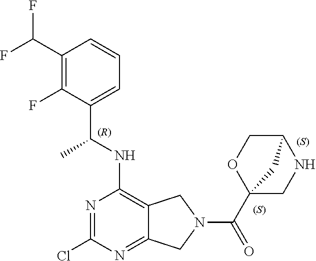

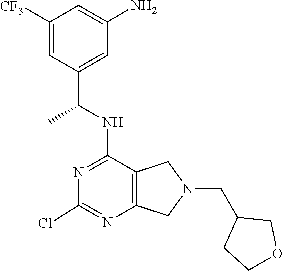 [SMILES: C[C@@H](Nc1nc(Cl)nc2c1CN(CC1CCOC1)C2)c1cc(N)cc(C(F)(F)F)c1]

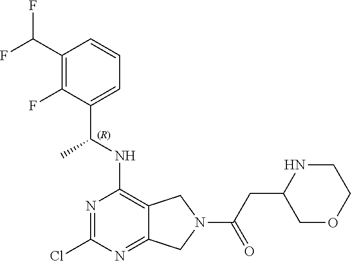

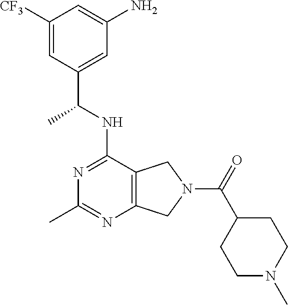 [SMILES: Cc1nc2c(c(N[C@H](C)c3cc(N)cc(C(F)(F)F)c3)n1)CN(C(=O)C1CCN(C)CC1)C2]

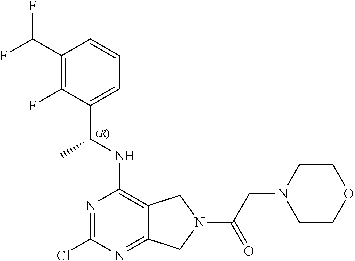

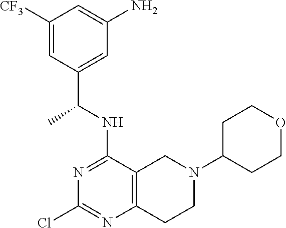 [SMILES: C[C@@H](Nc1nc(Cl)nc2c1CN(C1CCOCC1)CC2)c1cc(N)cc(C(F)(F)F)c1]

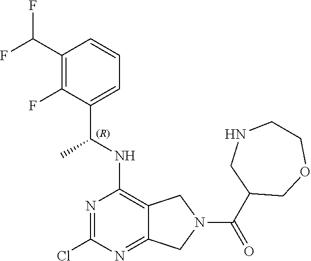

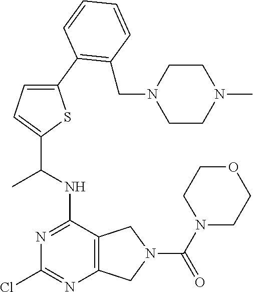 [SMILES: CC(Nc1nc(Cl)nc2c1CN(C(=O)N1CCOCC1)C2)c1ccc(-c2ccccc2CN2CCN(C)CC2)s1]

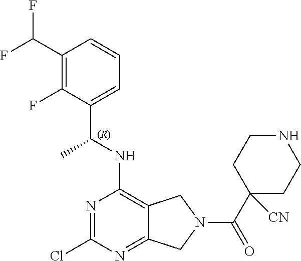

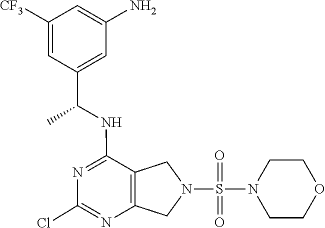 [SMILES: C[C@@H](Nc1nc(Cl)nc2c1CN(S(=O)(=O)N1CCOCC1)C2)c1cc(N)cc(C(F)(F)F)c1]

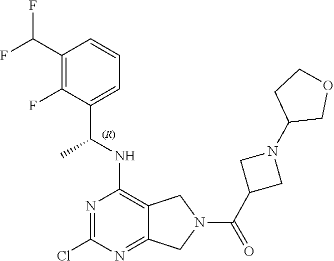

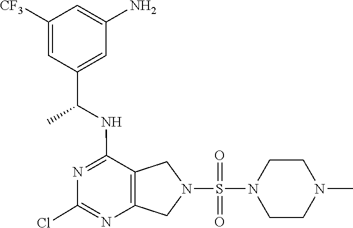 [SMILES: C[C@@H](Nc1nc(Cl)nc2c1CN(S(=O)(=O)N1CCN(C)CC1)C2)c1cc(N)cc(C(F)(F)F)c1]

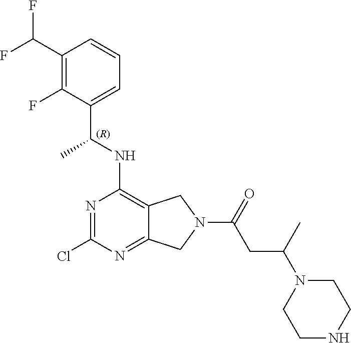

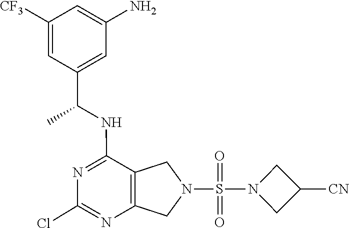 [SMILES: C[C@@H](Nc1nc(Cl)nc2c1CN(S(=O)(=O)N1CC(C#N)C1)C2)c1cc(N)cc(C(F)(F)F)c1]

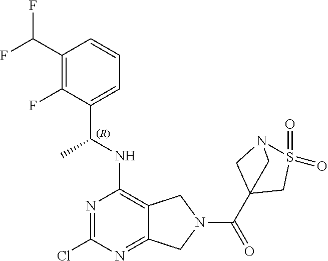

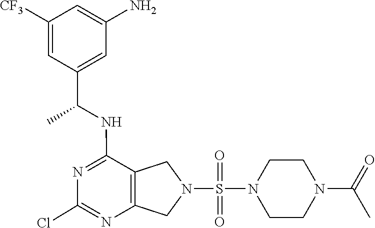 [SMILES: CC(=O)N1CCN(S(=O)(=O)N2Cc3nc(Cl)nc(N[C@H](C)c4cc(N)cc(C(F)(F)F)c4)c3C2)CC1]

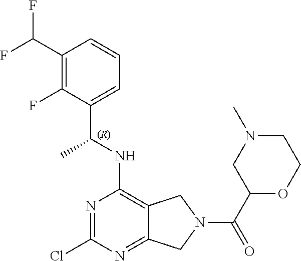

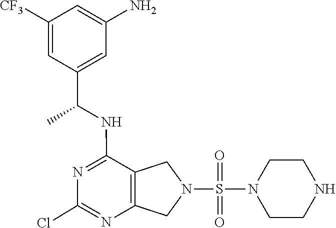 [SMILES: C[C@@H](Nc1nc(Cl)nc2c1CN(S(=O)(=O)N1CCNCC1)C2)c1cc(N)cc(C(F)(F)F)c1]

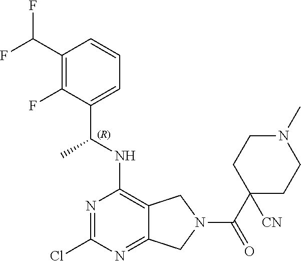

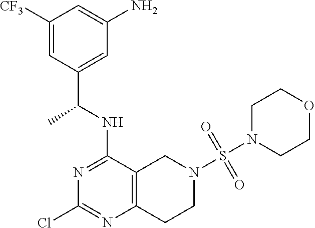 [SMILES: C[C@@H](Nc1nc(Cl)nc2c1CN(S(=O)(=O)N1CCOCC1)CC2)c1cc(N)cc(C(F)(F)F)c1]

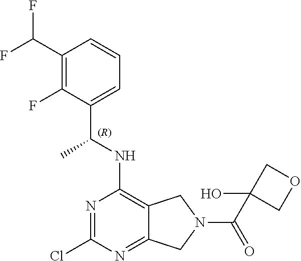

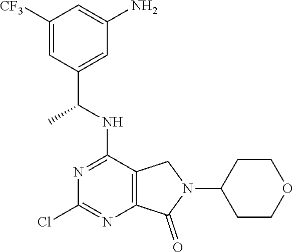 [SMILES: C[C@@H](Nc1nc(Cl)nc2c1CN(C1CCOCC1)C2=O)c1cc(N)cc(C(F)(F)F)c1]

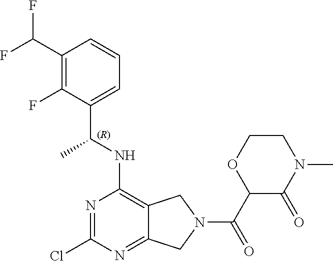

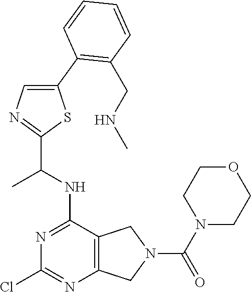 [SMILES: CNCc1ccccc1-c1cnc(C(C)Nc2nc(Cl)nc3c2CN(C(=O)N2CCOCC2)C3)s1]

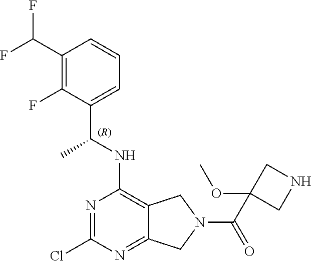

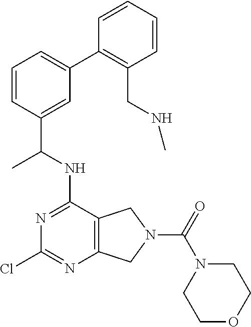 [SMILES: CNCc1ccccc1-c1cccc(C(C)Nc2nc(Cl)nc3c2CN(C(=O)N2CCOCC2)C3)c1]

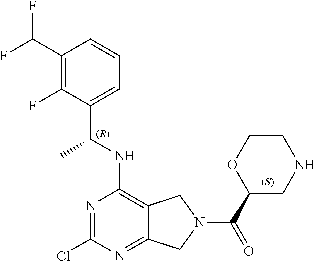

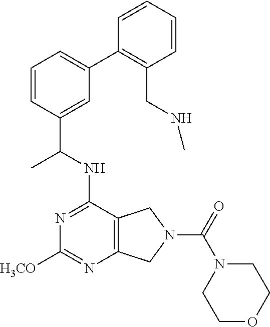 [SMILES: CNCc1ccccc1-c1cccc(C(C)Nc2nc(OC)nc3c2CN(C(=O)N2CCOCC2)C3)c1]

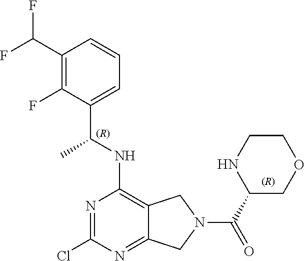

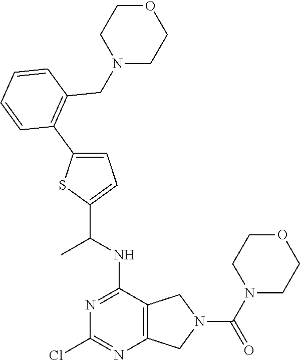 [SMILES: CC(Nc1nc(Cl)nc2c1CN(C(=O)N1CCOCC1)C2)c1ccc(-c2ccccc2CN2CCOCC2)s1]

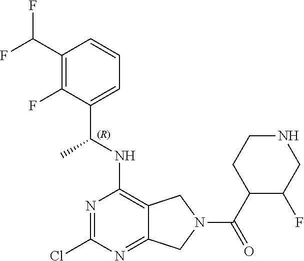

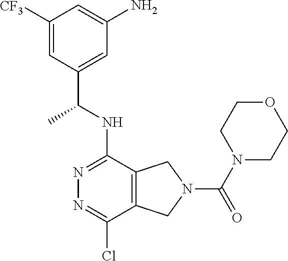 [SMILES: C[C@@H](Nc1nnc(Cl)c2c1CN(C(=O)N1CCOCC1)C2)c1cc(N)cc(C(F)(F)F)c1]

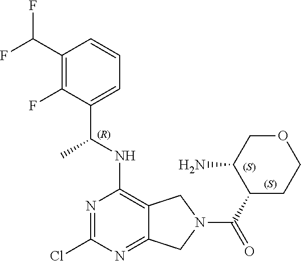

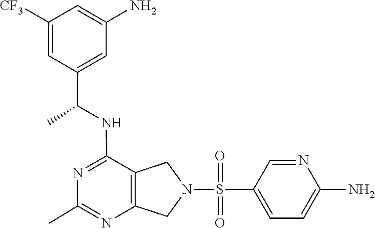 [SMILES: Cc1nc2c(c(N[C@H](C)c3cc(N)cc(C(F)(F)F)c3)n1)CN(S(=O)(=O)c1ccc(N)nc1)C2]

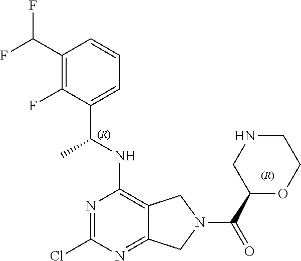

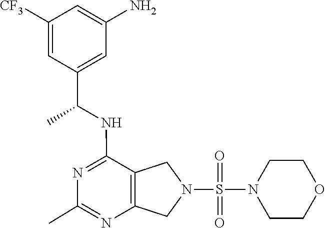 [SMILES: Cc1nc2c(c(N[C@H](C)c3cc(N)cc(C(F)(F)F)c3)n1)CN(S(=O)(=O)N1CCOCC1)C2]

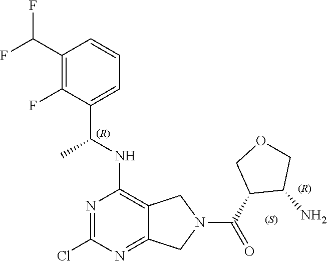

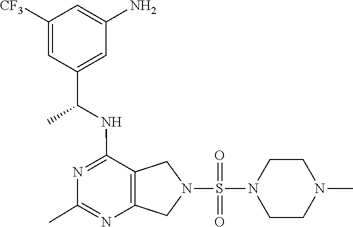 [SMILES: Cc1nc2c(c(N[C@H](C)c3cc(N)cc(C(F)(F)F)c3)n1)CN(S(=O)(=O)N1CCN(C)CC1)C2]

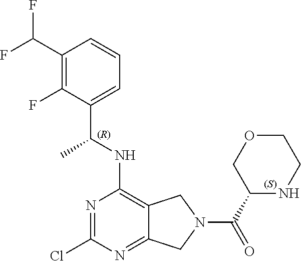

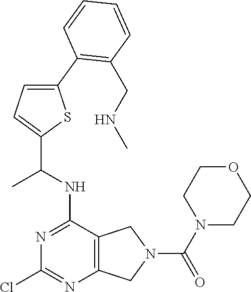 [SMILES: CNCc1ccccc1-c1ccc(C(C)Nc2nc(Cl)nc3c2CN(C(=O)N2CCOCC2)C3)s1]

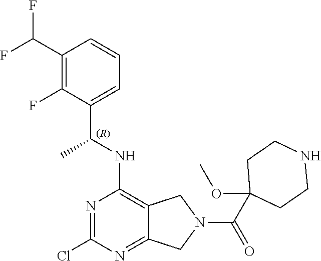

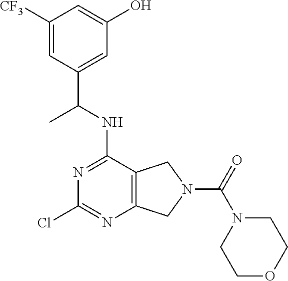 [SMILES: CC(Nc1nc(Cl)nc2c1CN(C(=O)N1CCOCC1)C2)c1cc(O)cc(C(F)(F)F)c1]

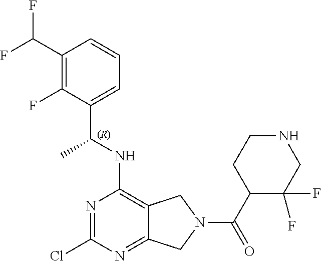

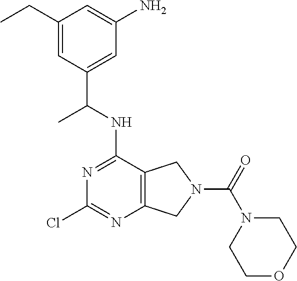 [SMILES: CCc1cc(N)cc(C(C)Nc2nc(Cl)nc3c2CN(C(=O)N2CCOCC2)C3)c1]

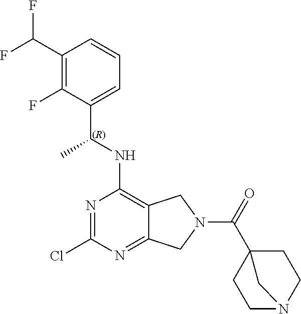

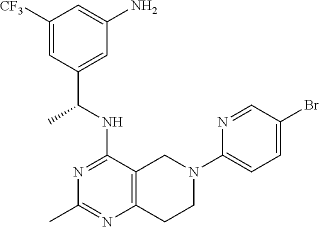 [SMILES: Cc1nc2c(c(N[C@H](C)c3cc(N)cc(C(F)(F)F)c3)n1)CN(c1ccc(Br)cn1)CC2]

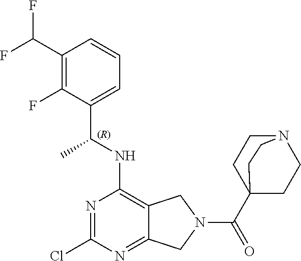

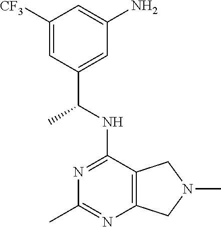 [SMILES: Cc1nc2c(c(N[C@H](C)c3cc(N)cc(C(F)(F)F)c3)n1)CN(C)C2]

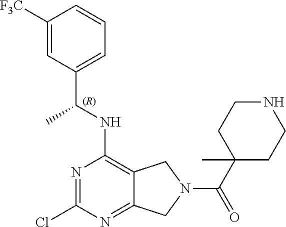

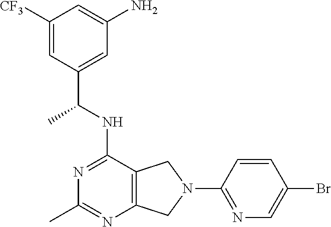 [SMILES: Cc1nc2c(c(N[C@H](C)c3cc(N)cc(C(F)(F)F)c3)n1)CN(c1ccc(Br)cn1)C2]

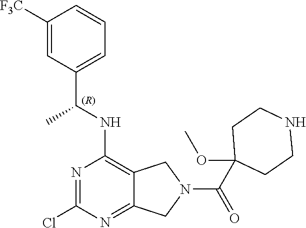

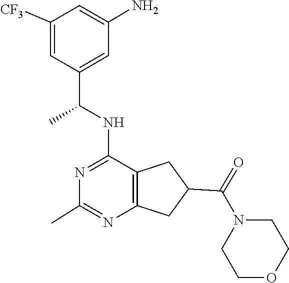 [SMILES: Cc1nc2c(c(N[C@H](C)c3cc(N)cc(C(F)(F)F)c3)n1)CC(C(=O)N1CCOCC1)C2]

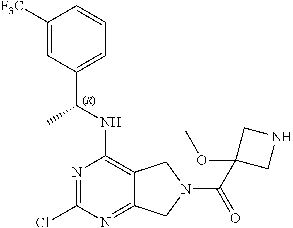

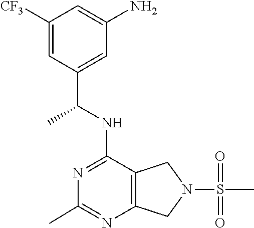 [SMILES: Cc1nc2c(c(N[C@H](C)c3cc(N)cc(C(F)(F)F)c3)n1)CN(S(C)(=O)=O)C2]

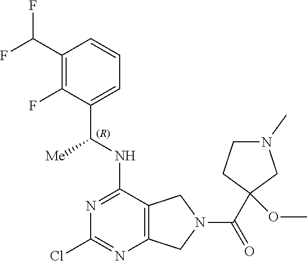

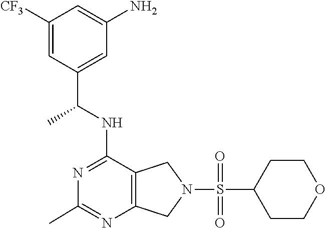 [SMILES: Cc1nc2c(c(N[C@H](C)c3cc(N)cc(C(F)(F)F)c3)n1)CN(S(=O)(=O)C1CCOCC1)C2]

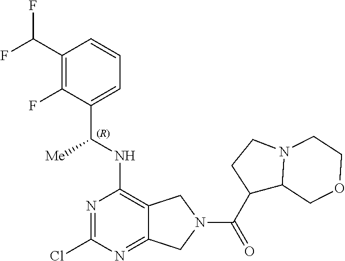

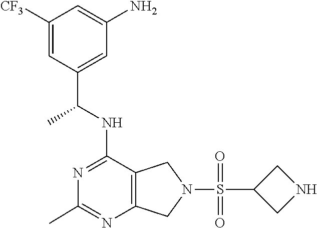 [SMILES: Cc1nc2c(c(N[C@H](C)c3cc(N)cc(C(F)(F)F)c3)n1)CN(S(=O)(=O)C1CNC1)C2]

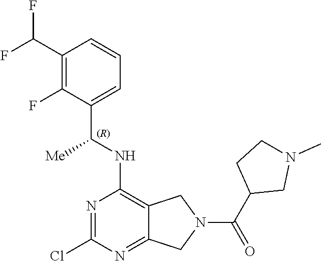

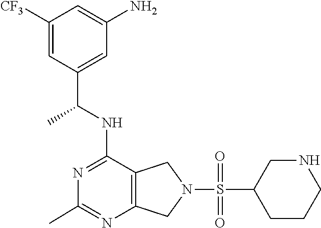 [SMILES: Cc1nc2c(c(N[C@H](C)c3cc(N)cc(C(F)(F)F)c3)n1)CN(S(=O)(=O)C1CCCNC1)C2]

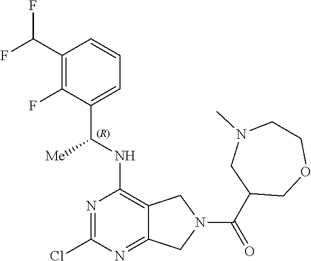

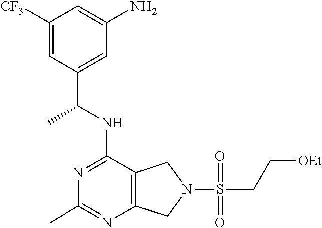 [SMILES: CCOCCS(=O)(=O)N1Cc2nc(C)nc(N[C@H](C)c3cc(N)cc(C(F)(F)F)c3)c2C1]

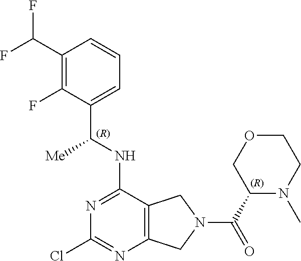

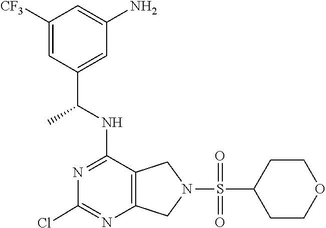 [SMILES: C[C@@H](Nc1nc(Cl)nc2c1CN(S(=O)(=O)C1CCOCC1)C2)c1cc(N)cc(C(F)(F)F)c1]

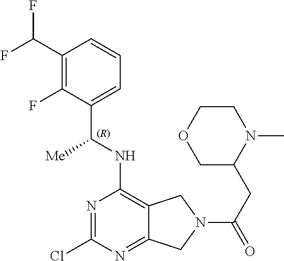

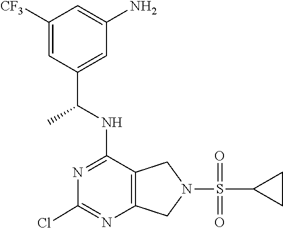 [SMILES: C[C@@H](Nc1nc(Cl)nc2c1CN(S(=O)(=O)C1CC1)C2)c1cc(N)cc(C(F)(F)F)c1]

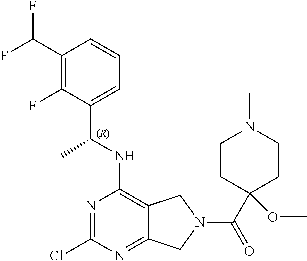

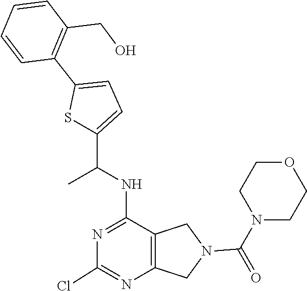 [SMILES: CC(Nc1nc(Cl)nc2c1CN(C(=O)N1CCOCC1)C2)c1ccc(-c2ccccc2CO)s1]

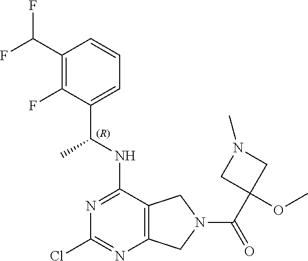

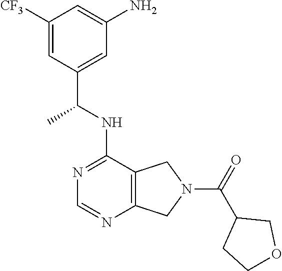 [SMILES: C[C@@H](Nc1ncnc2c1CN(C(=O)C1CCOC1)C2)c1cc(N)cc(C(F)(F)F)c1]

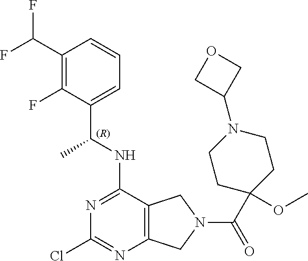

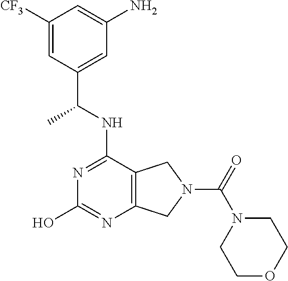 [SMILES: C[C@@H](Nc1nc(O)nc2c1CN(C(=O)N1CCOCC1)C2)c1cc(N)cc(C(F)(F)F)c1]

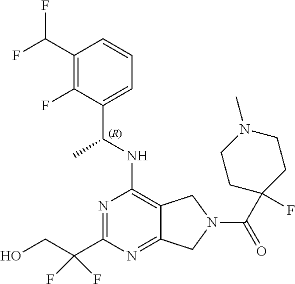

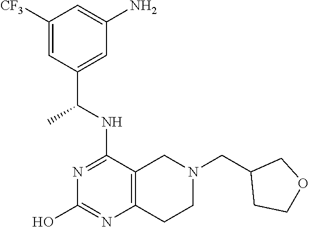 [SMILES: C[C@@H](Nc1nc(O)nc2c1CN(CC1CCOC1)CC2)c1cc(N)cc(C(F)(F)F)c1]

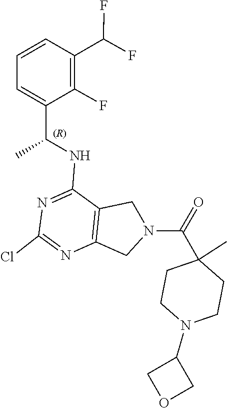

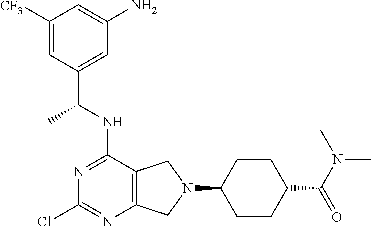

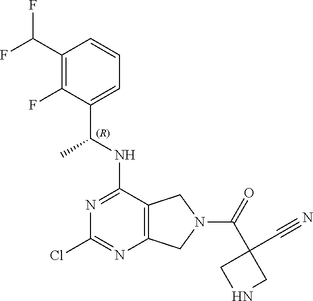

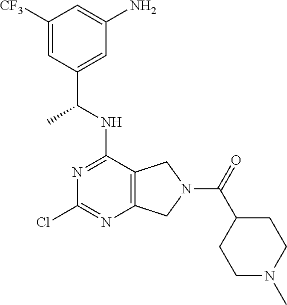 [SMILES: C[C@@H](Nc1nc(Cl)nc2c1CN(C(=O)C1CCN(C)CC1)C2)c1cc(N)cc(C(F)(F)F)c1]

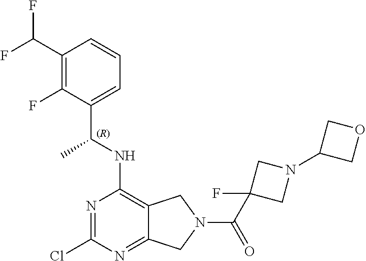

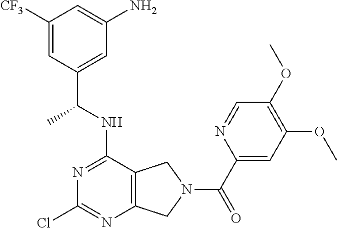 [SMILES: COc1cnc(C(=O)N2Cc3nc(Cl)nc(N[C@H](C)c4cc(N)cc(C(F)(F)F)c4)c3C2)cc1OC]

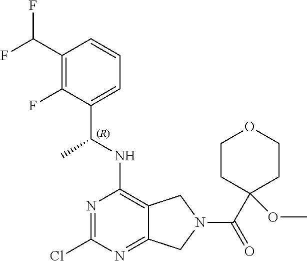

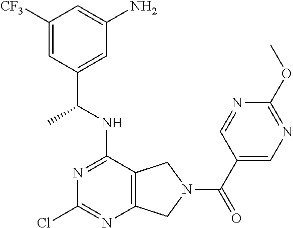 [SMILES: COc1ncc(C(=O)N2Cc3nc(Cl)nc(N[C@H](C)c4cc(N)cc(C(F)(F)F)c4)c3C2)cn1]

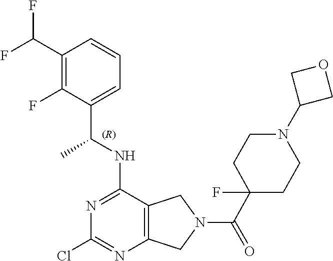

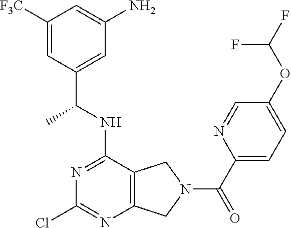 [SMILES: C[C@@H](Nc1nc(Cl)nc2c1CN(C(=O)c1ccc(OC(F)F)cn1)C2)c1cc(N)cc(C(F)(F)F)c1]

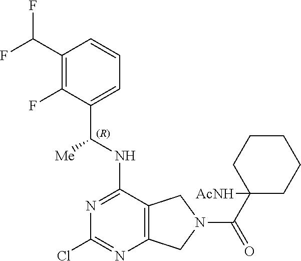

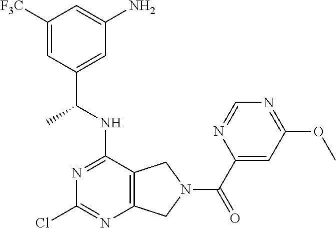 [SMILES: COc1cc(C(=O)N2Cc3nc(Cl)nc(N[C@H](C)c4cc(N)cc(C(F)(F)F)c4)c3C2)ncn1]

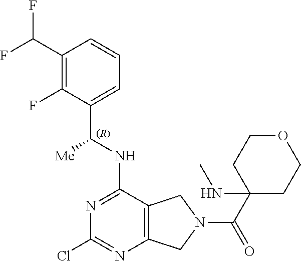

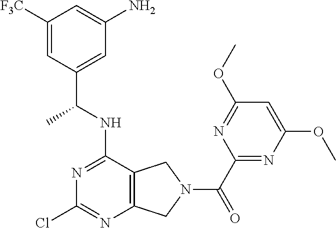 [SMILES: COc1cc(OC)nc(C(=O)N2Cc3nc(Cl)nc(N[C@H](C)c4cc(N)cc(C(F)(F)F)c4)c3C2)n1]

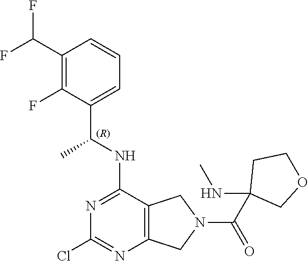

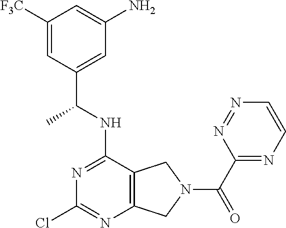 [SMILES: C[C@@H](Nc1nc(Cl)nc2c1CN(C(=O)c1nccnn1)C2)c1cc(N)cc(C(F)(F)F)c1]

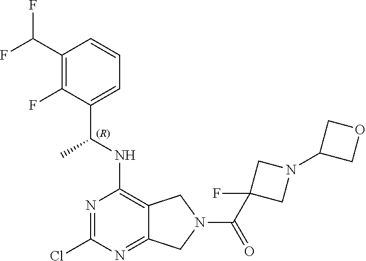

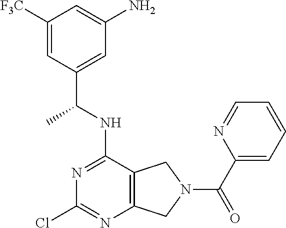 [SMILES: C[C@@H](Nc1nc(Cl)nc2c1CN(C(=O)c1ccccn1)C2)c1cc(N)cc(C(F)(F)F)c1]

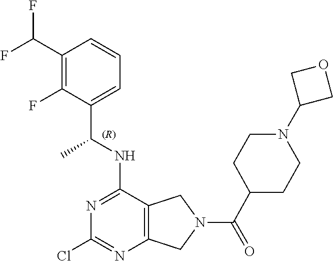

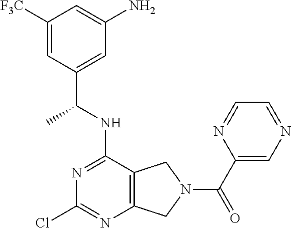 [SMILES: C[C@@H](Nc1nc(Cl)nc2c1CN(C(=O)c1cnccn1)C2)c1cc(N)cc(C(F)(F)F)c1]

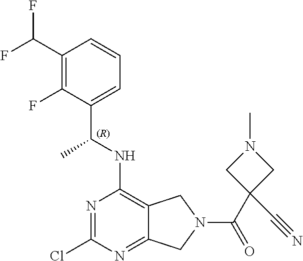

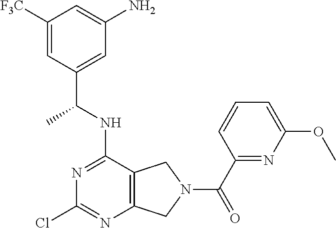 [SMILES: COc1cccc(C(=O)N2Cc3nc(Cl)nc(N[C@H](C)c4cc(N)cc(C(F)(F)F)c4)c3C2)n1]

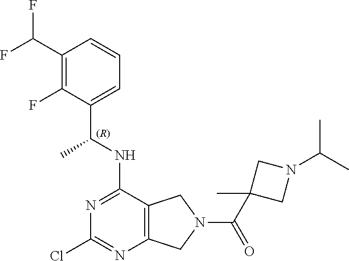

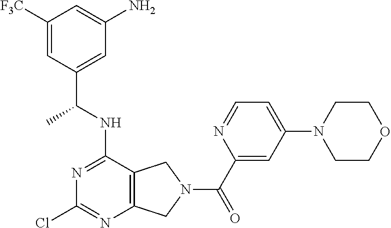 [SMILES: C[C@@H](Nc1nc(Cl)nc2c1CN(C(=O)c1cc(N3CCOCC3)ccn1)C2)c1cc(N)cc(C(F)(F)F)c1]

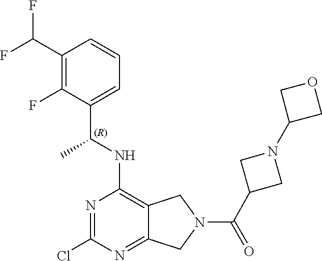

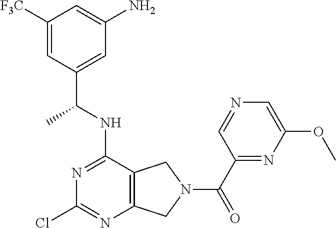 [SMILES: COc1cncc(C(=O)N2Cc3nc(Cl)nc(N[C@H](C)c4cc(N)cc(C(F)(F)F)c4)c3C2)n1]

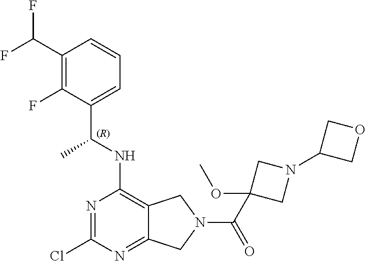

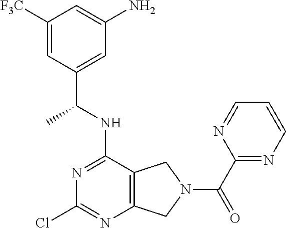 [SMILES: C[C@@H](Nc1nc(Cl)nc2c1CN(C(=O)c1ncccn1)C2)c1cc(N)cc(C(F)(F)F)c1]

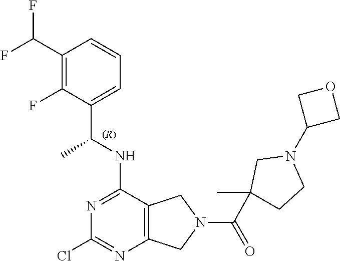

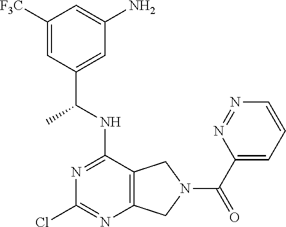 [SMILES: C[C@@H](Nc1nc(Cl)nc2c1CN(C(=O)c1cccnn1)C2)c1cc(N)cc(C(F)(F)F)c1]

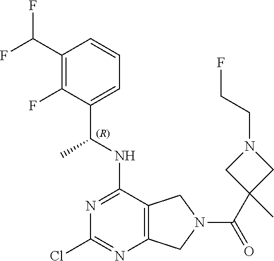

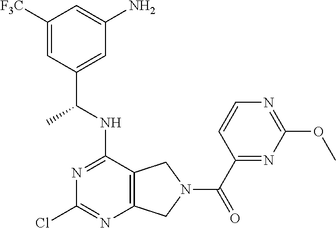 [SMILES: COc1nccc(C(=O)N2Cc3nc(Cl)nc(N[C@H](C)c4cc(N)cc(C(F)(F)F)c4)c3C2)n1]

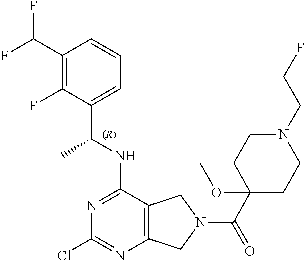

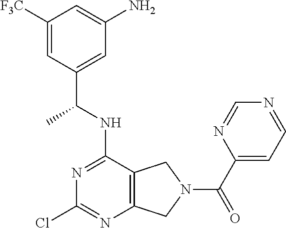 [SMILES: C[C@@H](Nc1nc(Cl)nc2c1CN(C(=O)c1ccncn1)C2)c1cc(N)cc(C(F)(F)F)c1]

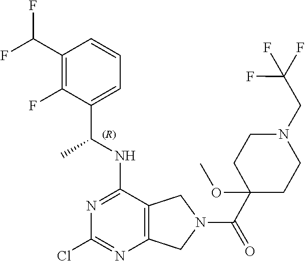

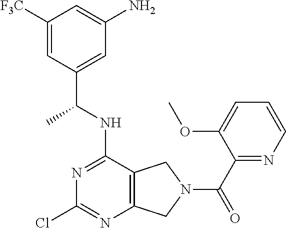 [SMILES: COc1cccnc1C(=O)N1Cc2nc(Cl)nc(N[C@H](C)c3cc(N)cc(C(F)(F)F)c3)c2C1]

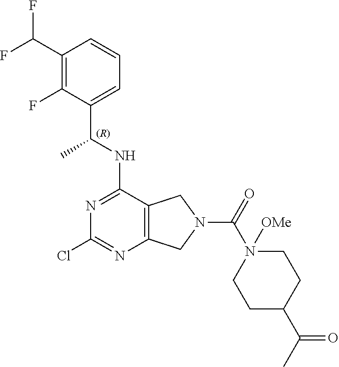

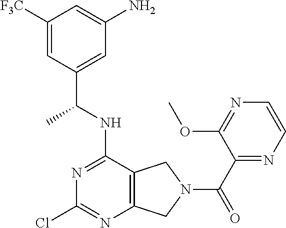 [SMILES: COc1nccnc1C(=O)N1Cc2nc(Cl)nc(N[C@H](C)c3cc(N)cc(C(F)(F)F)c3)c2C1]

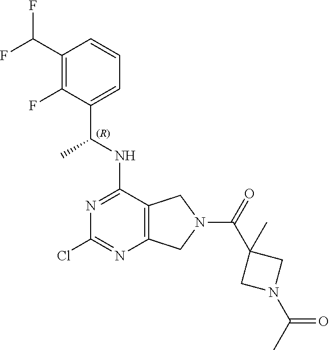

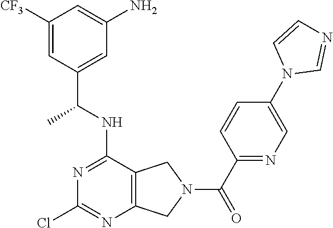 [SMILES: C[C@@H](Nc1nc(Cl)nc2c1CN(C(=O)c1ccc(-n3ccnc3)cn1)C2)c1cc(N)cc(C(F)(F)F)c1]

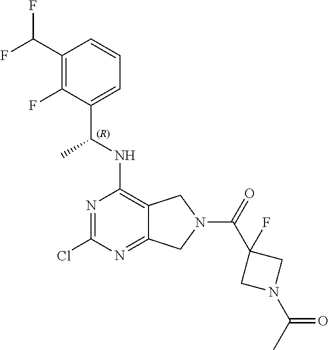

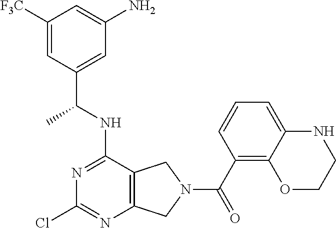 [SMILES: C[C@@H](Nc1nc(Cl)nc2c1CN(C(=O)c1cccc3c1OCCN3)C2)c1cc(N)cc(C(F)(F)F)c1]

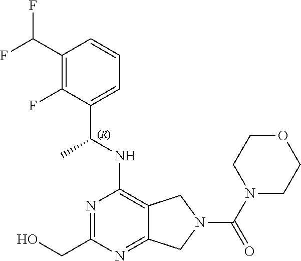

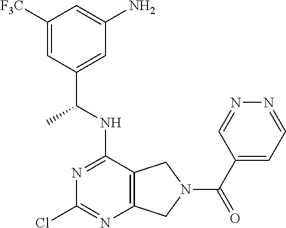 [SMILES: C[C@@H](Nc1nc(Cl)nc2c1CN(C(=O)c1ccnnc1)C2)c1cc(N)cc(C(F)(F)F)c1]

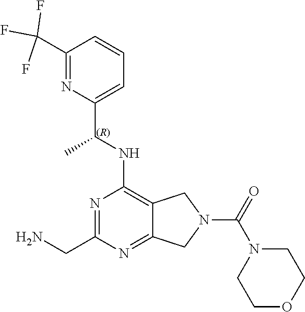

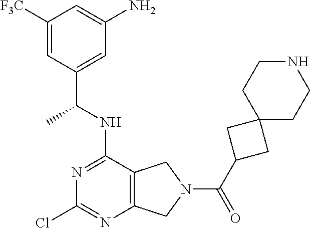 [SMILES: C[C@@H](Nc1nc(Cl)nc2c1CN(C(=O)C1CC3(CCNCC3)C1)C2)c1cc(N)cc(C(F)(F)F)c1]

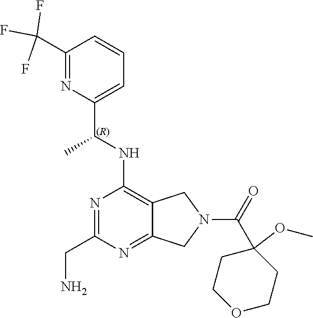

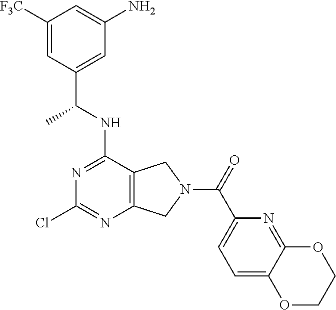 [SMILES: C[C@@H](Nc1nc(Cl)nc2c1CN(C(=O)c1ccc3c(n1)OCCO3)C2)c1cc(N)cc(C(F)(F)F)c1]

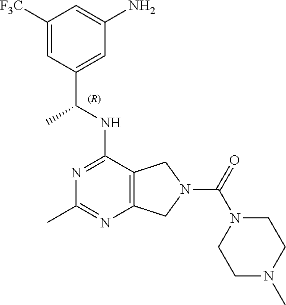

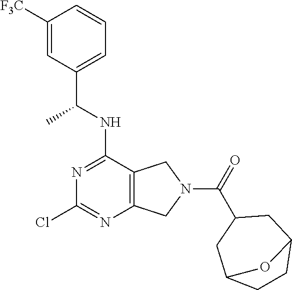 [SMILES: C[C@@H](Nc1nc(Cl)nc2c1CN(C(=O)C1CC3CCC(C1)O3)C2)c1cccc(C(F)(F)F)c1]

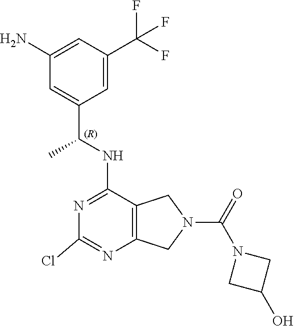

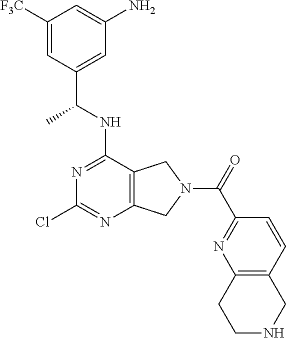 [SMILES: C[C@@H](Nc1nc(Cl)nc2c1CN(C(=O)c1ccc3c(n1)CCNC3)C2)c1cc(N)cc(C(F)(F)F)c1]

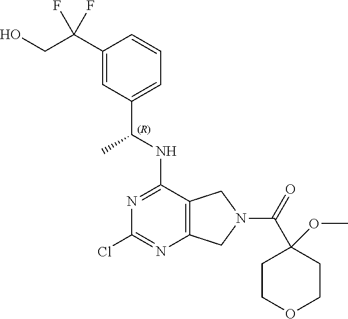

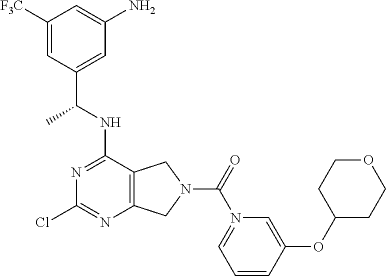

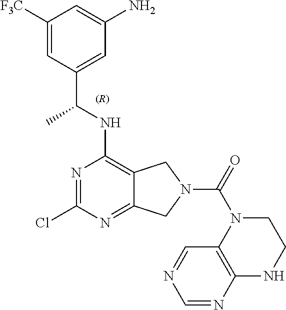

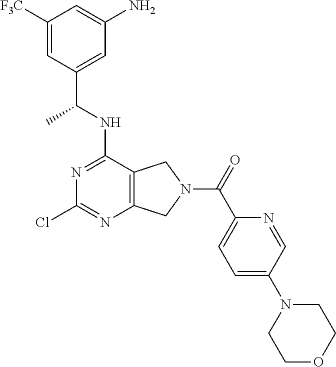 [SMILES: C[C@@H](Nc1nc(Cl)nc2c1CN(C(=O)c1ccc(N3CCOCC3)cn1)C2)c1cc(N)cc(C(F)(F)F)c1]

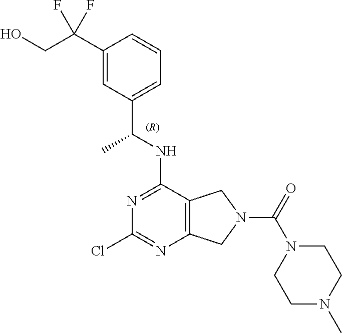

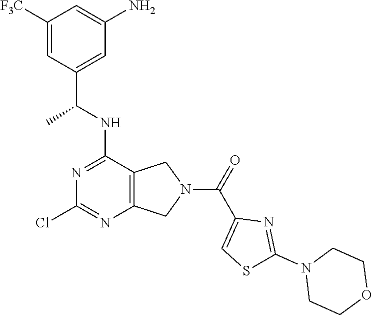 [SMILES: C[C@@H](Nc1nc(Cl)nc2c1CN(C(=O)c1csc(N3CCOCC3)n1)C2)c1cc(N)cc(C(F)(F)F)c1]

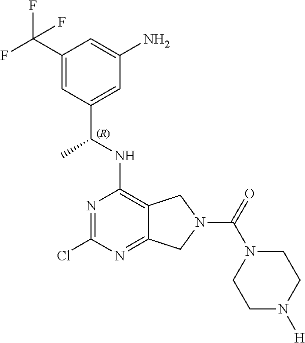

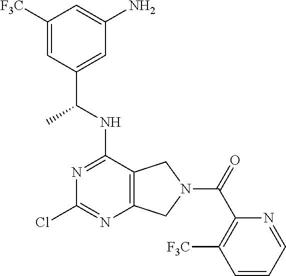 [SMILES: C[C@@H](Nc1nc(Cl)nc2c1CN(C(=O)c1ncccc1C(F)(F)F)C2)c1cc(N)cc(C(F)(F)F)c1]

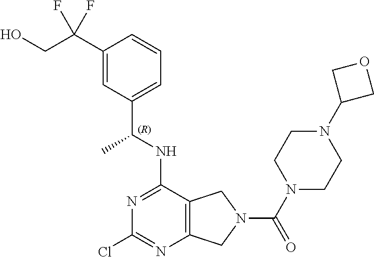

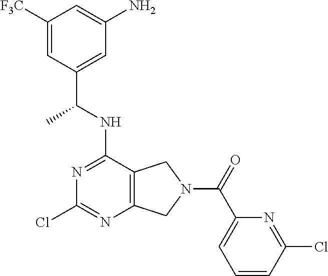 [SMILES: C[C@@H](Nc1nc(Cl)nc2c1CN(C(=O)c1cccc(Cl)n1)C2)c1cc(N)cc(C(F)(F)F)c1]

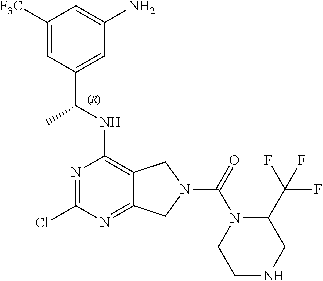

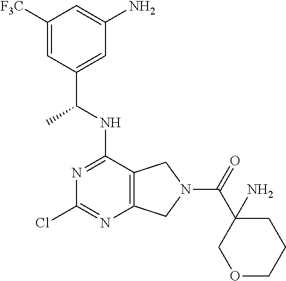 [SMILES: C[C@@H](Nc1nc(Cl)nc2c1CN(C(=O)C1(N)CCCOC1)C2)c1cc(N)cc(C(F)(F)F)c1]

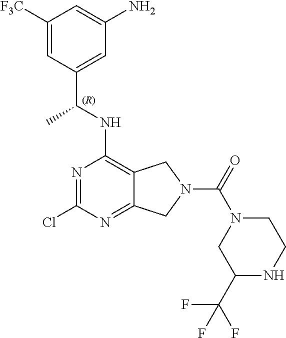

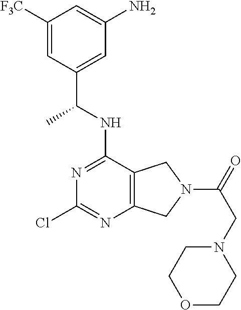 [SMILES: C[C@@H](Nc1nc(Cl)nc2c1CN(C(=O)CN1CCOCC1)C2)c1cc(N)cc(C(F)(F)F)c1]

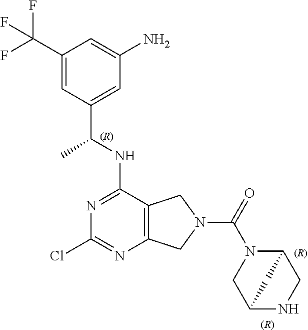

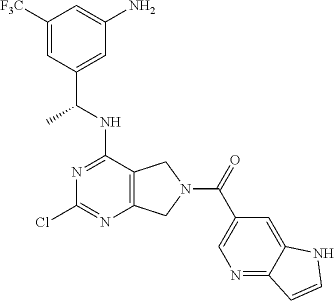 [SMILES: C[C@@H](Nc1nc(Cl)nc2c1CN(C(=O)c1cnc3cc[nH]c3c1)C2)c1cc(N)cc(C(F)(F)F)c1]

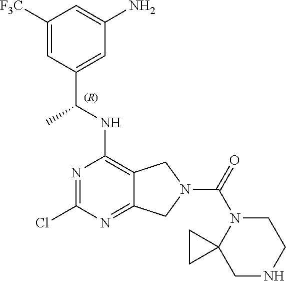

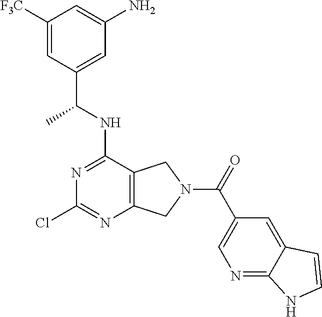 [SMILES: C[C@@H](Nc1nc(Cl)nc2c1CN(C(=O)c1cnc3[nH]ccc3c1)C2)c1cc(N)cc(C(F)(F)F)c1]

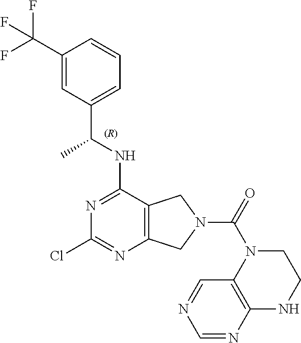

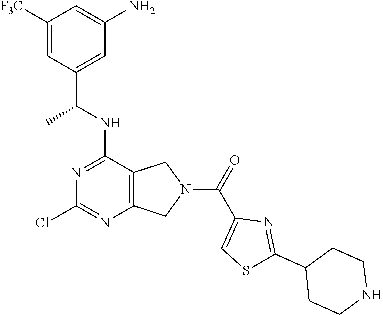 [SMILES: C[C@@H](Nc1nc(Cl)nc2c1CN(C(=O)c1csc(C3CCNCC3)n1)C2)c1cc(N)cc(C(F)(F)F)c1]

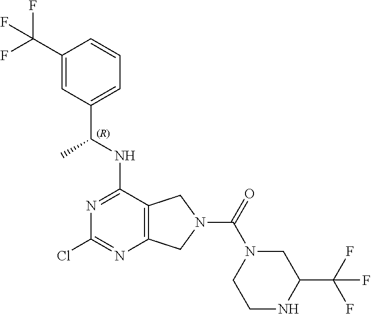

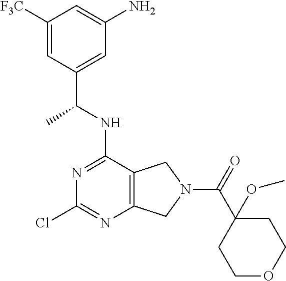 [SMILES: COC1(C(=O)N2Cc3nc(Cl)nc(N[C@H](C)c4cc(N)cc(C(F)(F)F)c4)c3C2)CCOCC1]

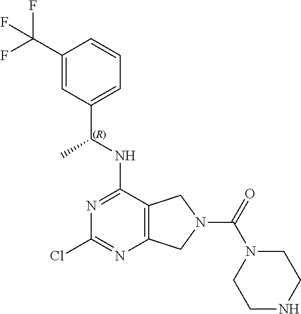

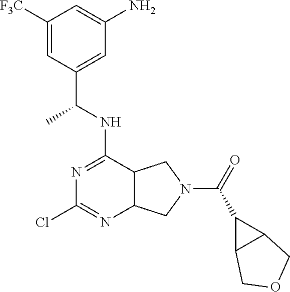

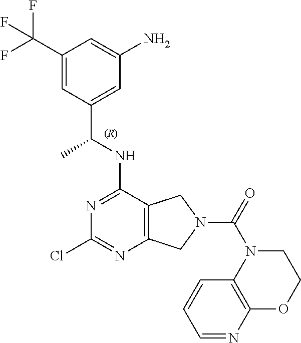

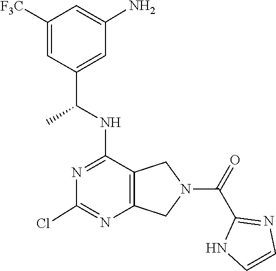 [SMILES: C[C@@H](Nc1nc(Cl)nc2c1CN(C(=O)c1ncc[nH]1)C2)c1cc(N)cc(C(F)(F)F)c1]

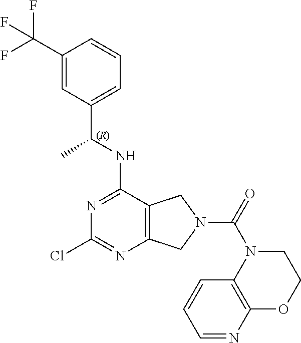

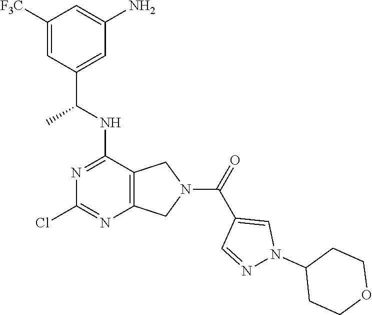 [SMILES: C[C@@H](Nc1nc(Cl)nc2c1CN(C(=O)c1cnn(C3CCOCC3)c1)C2)c1cc(N)cc(C(F)(F)F)c1]

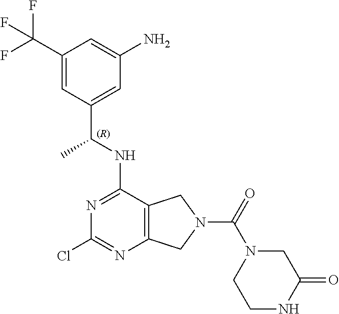

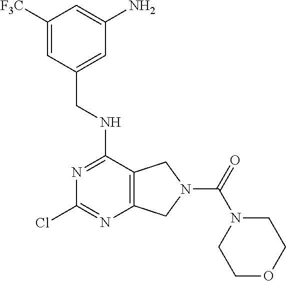 [SMILES: Nc1cc(CNc2nc(Cl)nc3c2CN(C(=O)N2CCOCC2)C3)cc(C(F)(F)F)c1]

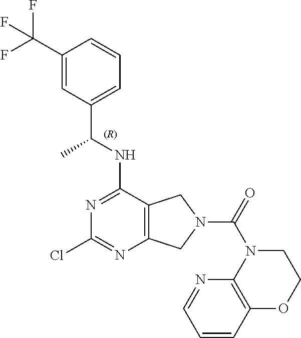

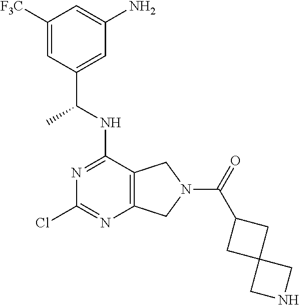 [SMILES: C[C@@H](Nc1nc(Cl)nc2c1CN(C(=O)C1CC3(CNC3)C1)C2)c1cc(N)cc(C(F)(F)F)c1]

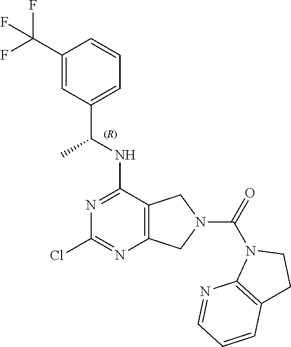

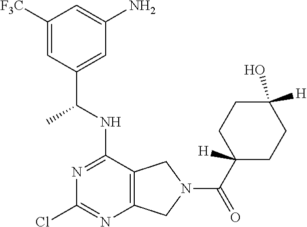 [SMILES: C[C@@H](Nc1nc(Cl)nc2c1CN(C(=O)[C@H]1CC[C@@H](O)CC1)C2)c1cc(N)cc(C(F)(F)F)c1]

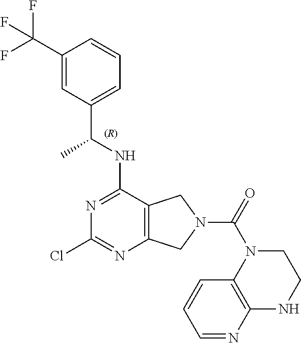

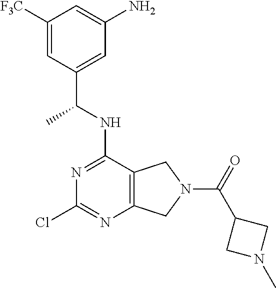 [SMILES: C[C@@H](Nc1nc(Cl)nc2c1CN(C(=O)C1CN(C)C1)C2)c1cc(N)cc(C(F)(F)F)c1]

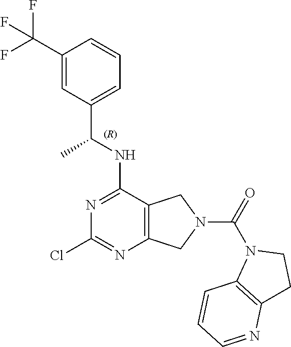

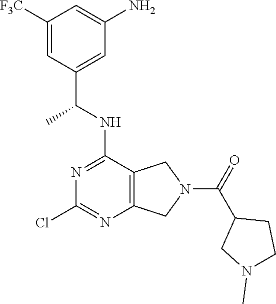 [SMILES: C[C@@H](Nc1nc(Cl)nc2c1CN(C(=O)C1CCN(C)C1)C2)c1cc(N)cc(C(F)(F)F)c1]

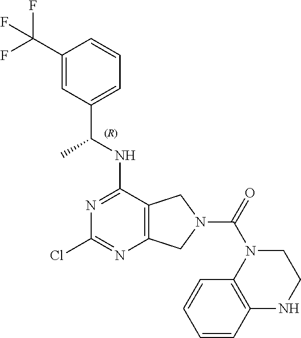

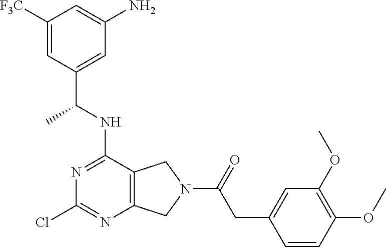 [SMILES: COc1ccc(CC(=O)N2Cc3nc(Cl)nc(N[C@H](C)c4cc(N)cc(C(F)(F)F)c4)c3C2)cc1OC]

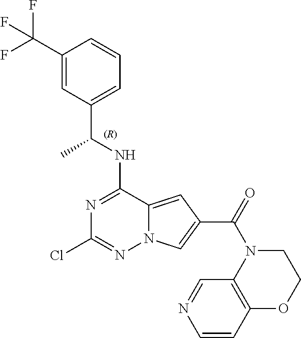

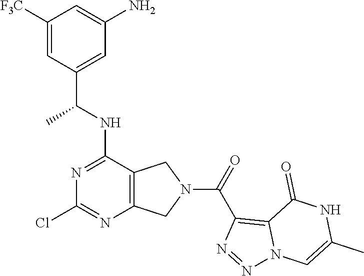 [SMILES: Cc1cn2nnc(C(=O)N3Cc4nc(Cl)nc(N[C@H](C)c5cc(N)cc(C(F)(F)F)c5)c4C3)c2c(=O)[nH]1]

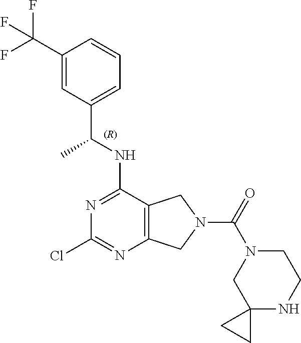

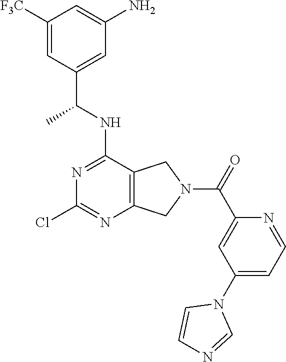 [SMILES: C[C@@H](Nc1nc(Cl)nc2c1CN(C(=O)c1cc(-n3ccnc3)ccn1)C2)c1cc(N)cc(C(F)(F)F)c1]

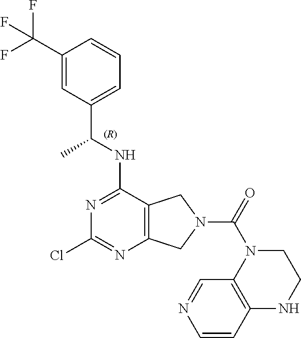

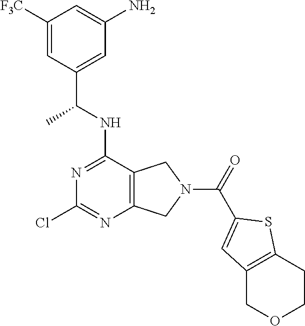 [SMILES: C[C@@H](Nc1nc(Cl)nc2c1CN(C(=O)c1cc3c(s1)CCOC3)C2)c1cc(N)cc(C(F)(F)F)c1]

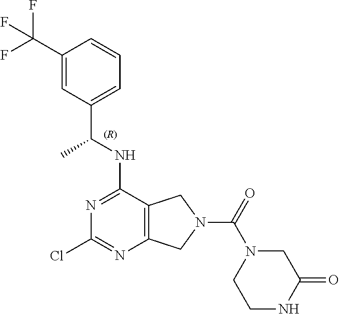

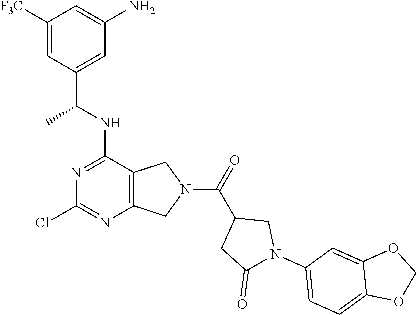 [SMILES: C[C@@H](Nc1nc(Cl)nc2c1CN(C(=O)C1CC(=O)N(c3ccc4c(c3)OCO4)C1)C2)c1cc(N)cc(C(F)(F)F)c1]

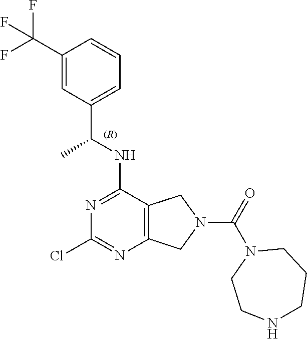

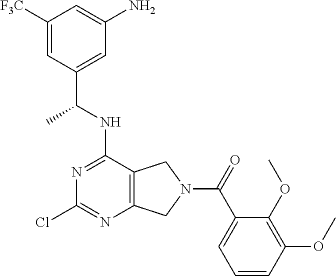 [SMILES: COc1cccc(C(=O)N2Cc3nc(Cl)nc(N[C@H](C)c4cc(N)cc(C(F)(F)F)c4)c3C2)c1OC]

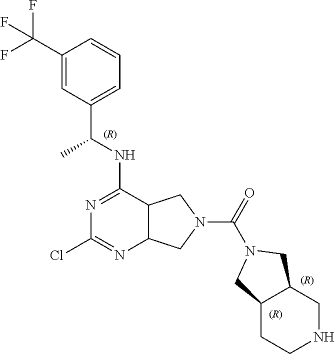

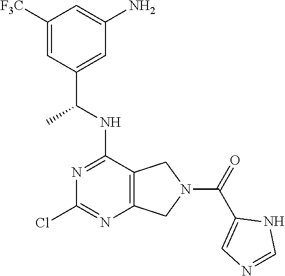 [SMILES: C[C@@H](Nc1nc(Cl)nc2c1CN(C(=O)c1cnc[nH]1)C2)c1cc(N)cc(C(F)(F)F)c1]

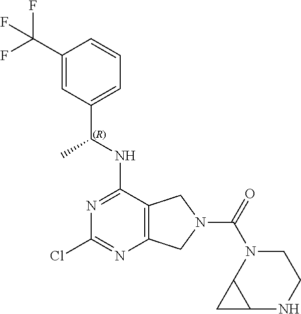

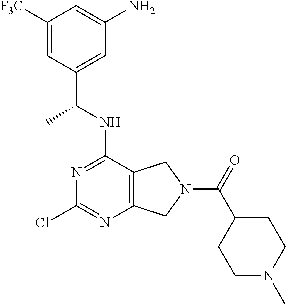 [SMILES: C[C@@H](Nc1nc(Cl)nc2c1CN(C(=O)C1CCN(C)CC1)C2)c1cc(N)cc(C(F)(F)F)c1]

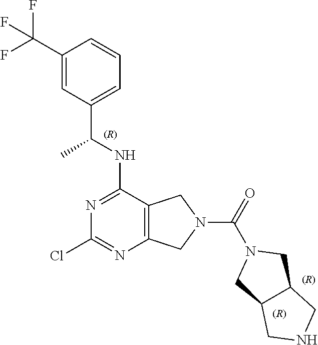

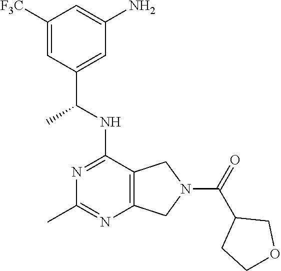 [SMILES: Cc1nc2c(c(N[C@H](C)c3cc(N)cc(C(F)(F)F)c3)n1)CN(C(=O)C1CCOC1)C2]

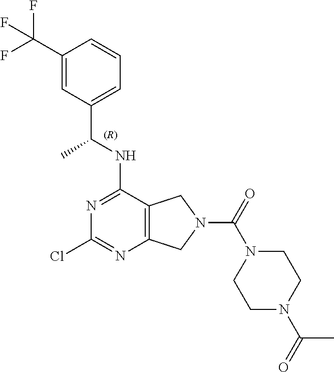

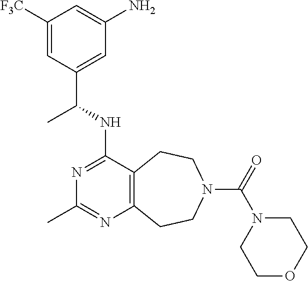 [SMILES: Cc1nc2c(c(N[C@H](C)c3cc(N)cc(C(F)(F)F)c3)n1)CCN(C(=O)N1CCOCC1)CC2]

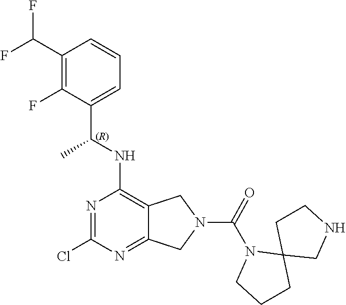

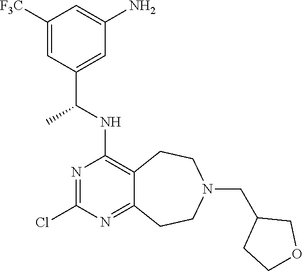 [SMILES: C[C@@H](Nc1nc(Cl)nc2c1CCN(CC1CCOC1)CC2)c1cc(N)cc(C(F)(F)F)c1]

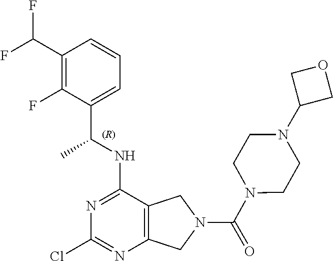

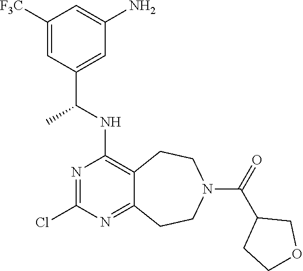 [SMILES: C[C@@H](Nc1nc(Cl)nc2c1CCN(C(=O)C1CCOC1)CC2)c1cc(N)cc(C(F)(F)F)c1]

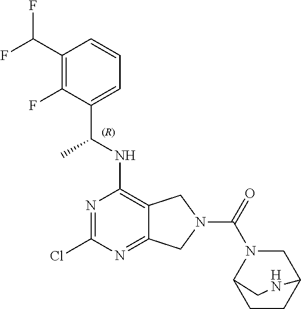

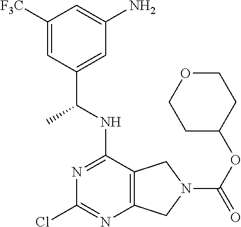 [SMILES: C[C@@H](Nc1nc(Cl)nc2c1CN(C(=O)OC1CCOCC1)C2)c1cc(N)cc(C(F)(F)F)c1]

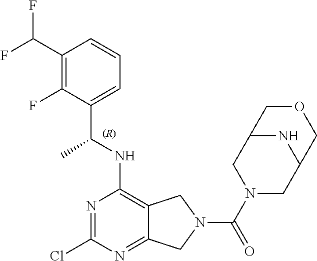

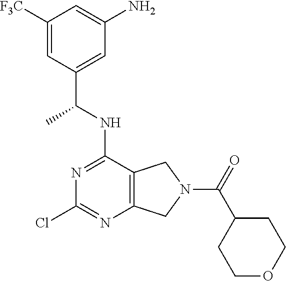 [SMILES: C[C@@H](Nc1nc(Cl)nc2c1CN(C(=O)C1CCOCC1)C2)c1cc(N)cc(C(F)(F)F)c1]

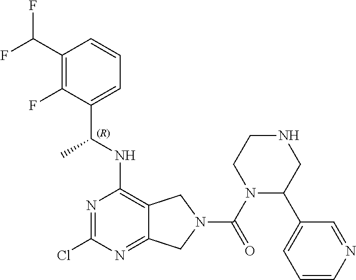

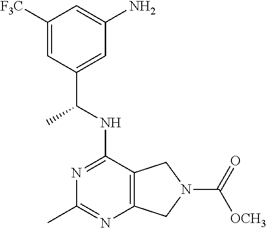 [SMILES: COC(=O)N1Cc2nc(C)nc(N[C@H](C)c3cc(N)cc(C(F)(F)F)c3)c2C1]

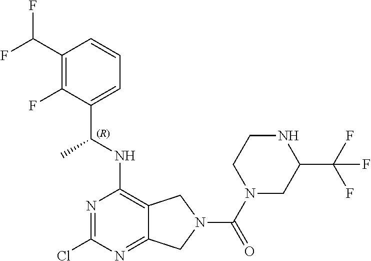

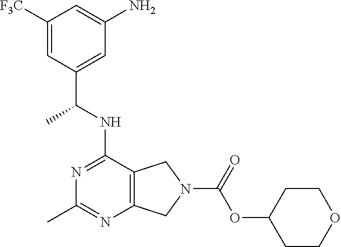 [SMILES: Cc1nc2c(c(N[C@H](C)c3cc(N)cc(C(F)(F)F)c3)n1)CN(C(=O)OC1CCOCC1)C2]

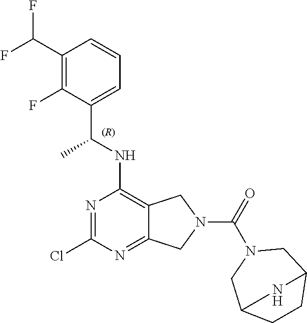

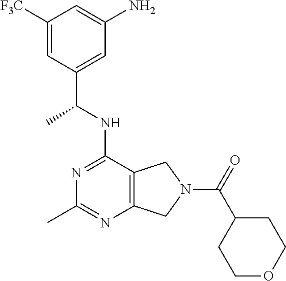 [SMILES: Cc1nc2c(c(N[C@H](C)c3cc(N)cc(C(F)(F)F)c3)n1)CN(C(=O)C1CCOCC1)C2]

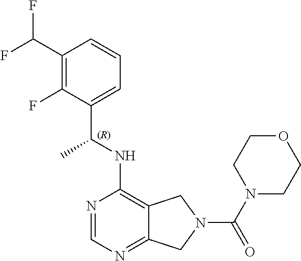

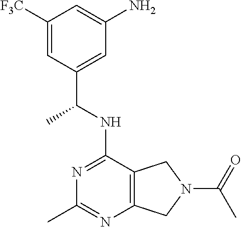 [SMILES: CC(=O)N1Cc2nc(C)nc(N[C@H](C)c3cc(N)cc(C(F)(F)F)c3)c2C1]

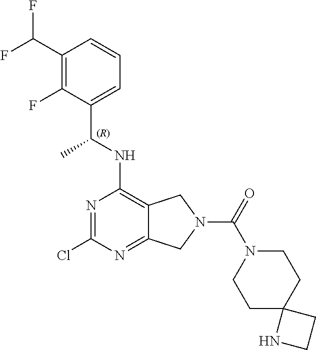

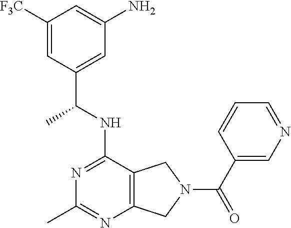 [SMILES: Cc1nc2c(c(N[C@H](C)c3cc(N)cc(C(F)(F)F)c3)n1)CN(C(=O)c1cccnc1)C2]

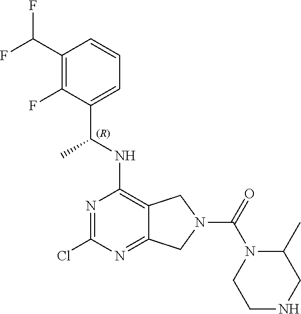

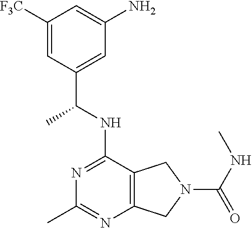 [SMILES: CNC(=O)N1Cc2nc(C)nc(N[C@H](C)c3cc(N)cc(C(F)(F)F)c3)c2C1]

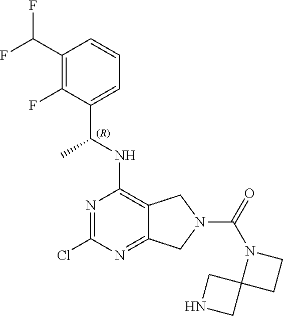

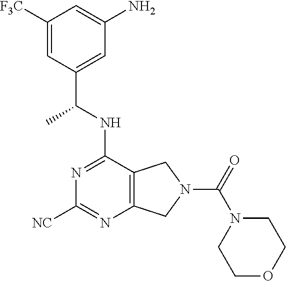 [SMILES: C[C@@H](Nc1nc(C#N)nc2c1CN(C(=O)N1CCOCC1)C2)c1cc(N)cc(C(F)(F)F)c1]

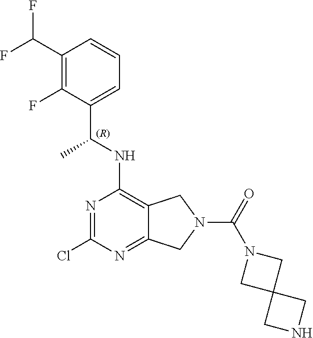

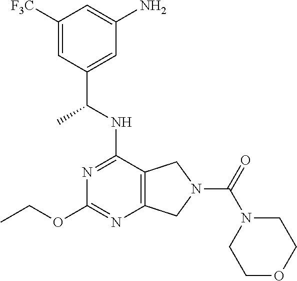 [SMILES: CCOc1nc2c(c(N[C@H](C)c3cc(N)cc(C(F)(F)F)c3)n1)CN(C(=O)N1CCOCC1)C2]

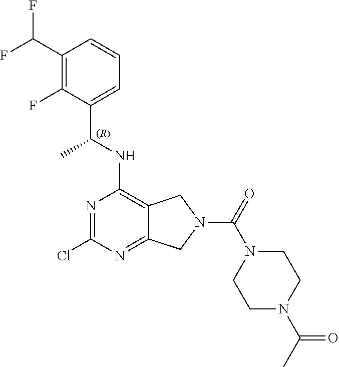

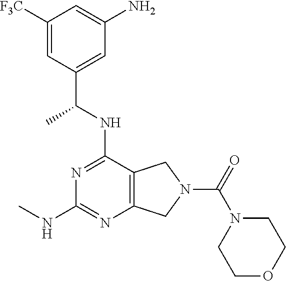 [SMILES: CNc1nc2c(c(N[C@H](C)c3cc(N)cc(C(F)(F)F)c3)n1)CN(C(=O)N1CCOCC1)C2]

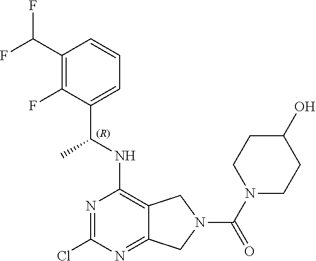

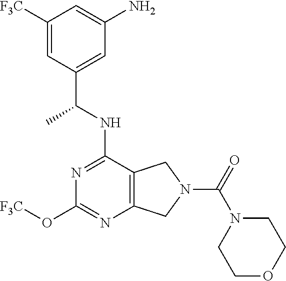 [SMILES: C[C@@H](Nc1nc(OC(F)(F)F)nc2c1CN(C(=O)N1CCOCC1)C2)c1cc(N)cc(C(F)(F)F)c1]

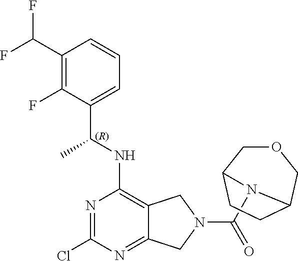

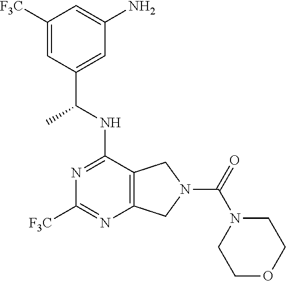 [SMILES: C[C@@H](Nc1nc(C(F)(F)F)nc2c1CN(C(=O)N1CCOCC1)C2)c1cc(N)cc(C(F)(F)F)c1]

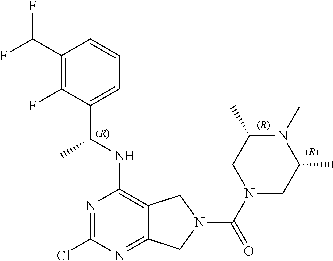

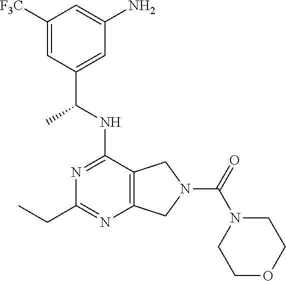 [SMILES: CCc1nc2c(c(N[C@H](C)c3cc(N)cc(C(F)(F)F)c3)n1)CN(C(=O)N1CCOCC1)C2]

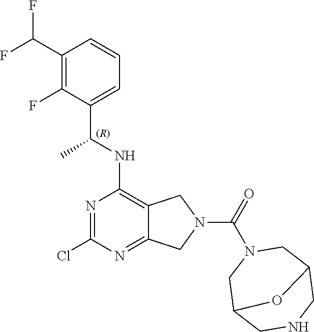

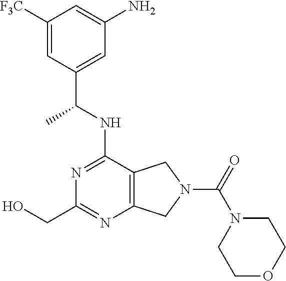 [SMILES: C[C@@H](Nc1nc(CO)nc2c1CN(C(=O)N1CCOCC1)C2)c1cc(N)cc(C(F)(F)F)c1]

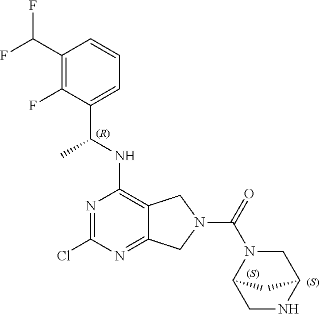

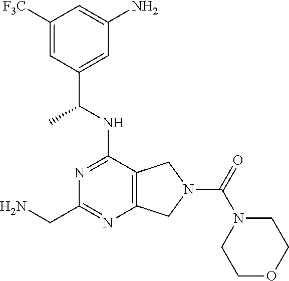 [SMILES: C[C@@H](Nc1nc(CN)nc2c1CN(C(=O)N1CCOCC1)C2)c1cc(N)cc(C(F)(F)F)c1]

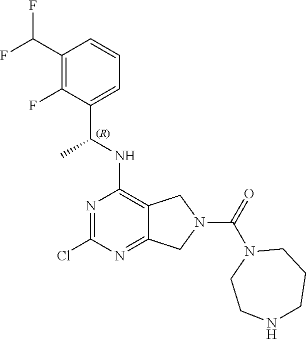

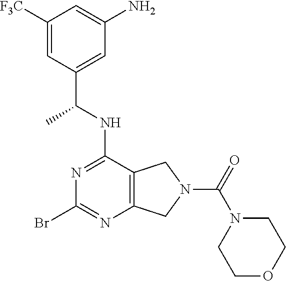 [SMILES: C[C@@H](Nc1nc(Br)nc2c1CN(C(=O)N1CCOCC1)C2)c1cc(N)cc(C(F)(F)F)c1]

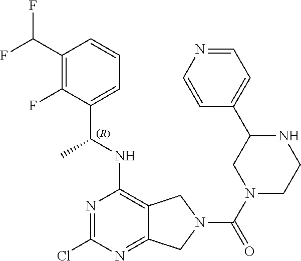

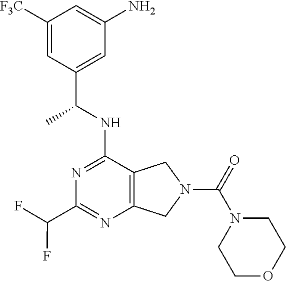 [SMILES: C[C@@H](Nc1nc(C(F)F)nc2c1CN(C(=O)N1CCOCC1)C2)c1cc(N)cc(C(F)(F)F)c1]

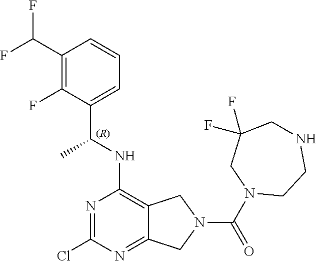

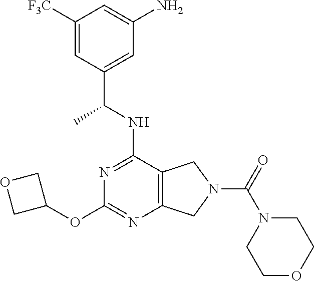 [SMILES: C[C@@H](Nc1nc(OC2COC2)nc2c1CN(C(=O)N1CCOCC1)C2)c1cc(N)cc(C(F)(F)F)c1]

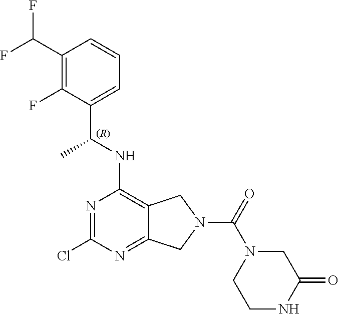

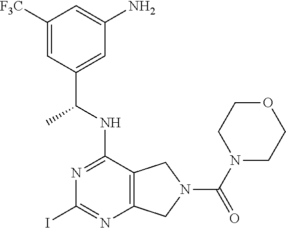 [SMILES: C[C@@H](Nc1nc(I)nc2c1CN(C(=O)N1CCOCC1)C2)c1cc(N)cc(C(F)(F)F)c1]

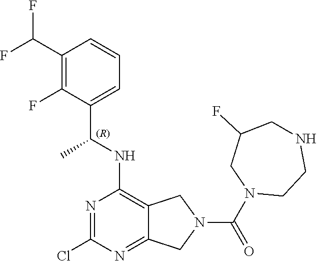

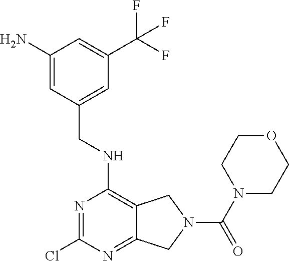 [SMILES: Nc1cc(CNc2nc(Cl)nc3c2CN(C(=O)N2CCOCC2)C3)cc(C(F)(F)F)c1]

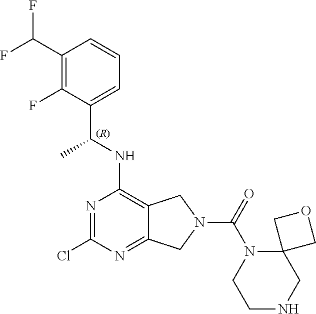

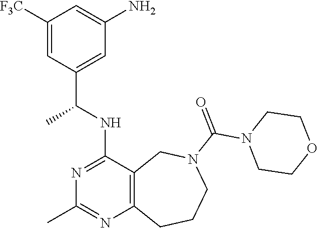 [SMILES: Cc1nc2c(c(N[C@H](C)c3cc(N)cc(C(F)(F)F)c3)n1)CN(C(=O)N1CCOCC1)CCC2]

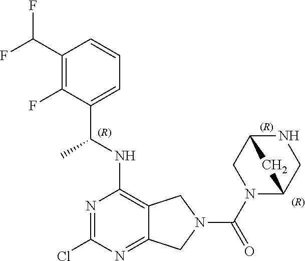

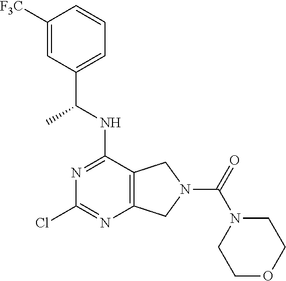 [SMILES: C[C@@H](Nc1nc(Cl)nc2c1CN(C(=O)N1CCOCC1)C2)c1cccc(C(F)(F)F)c1]

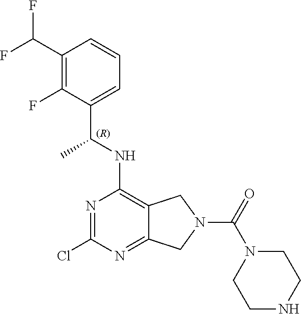

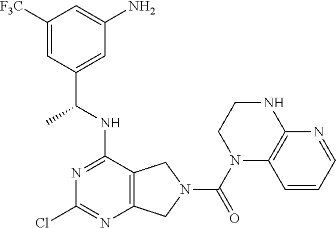 [SMILES: C[C@@H](Nc1nc(Cl)nc2c1CN(C(=O)N1CCNc3ncccc31)C2)c1cc(N)cc(C(F)(F)F)c1]

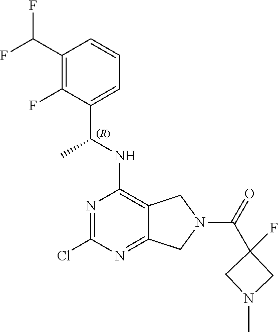

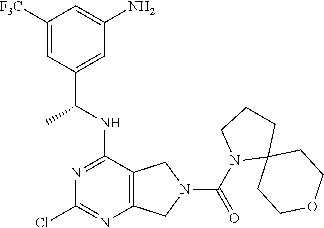 [SMILES: C[C@@H](Nc1nc(Cl)nc2c1CN(C(=O)N1CCCC13CCOCC3)C2)c1cc(N)cc(C(F)(F)F)c1]

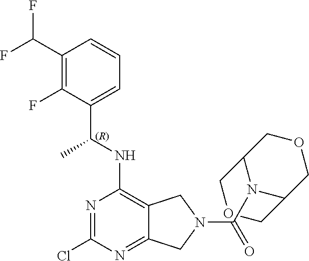

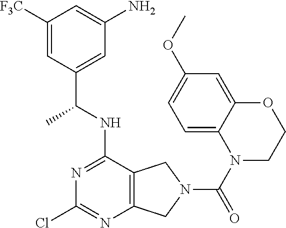 [SMILES: COc1ccc2c(c1)OCCN2C(=O)N1Cc2nc(Cl)nc(N[C@H](C)c3cc(N)cc(C(F)(F)F)c3)c2C1]

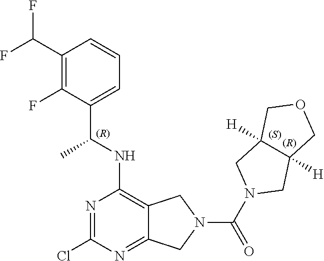

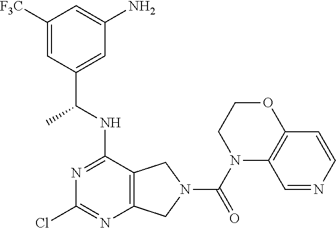 [SMILES: C[C@@H](Nc1nc(Cl)nc2c1CN(C(=O)N1CCOc3ccncc31)C2)c1cc(N)cc(C(F)(F)F)c1]

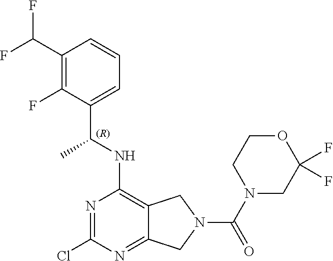

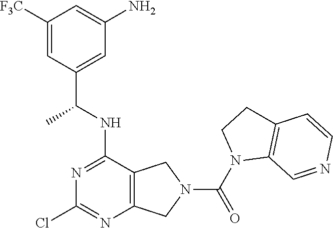 [SMILES: C[C@@H](Nc1nc(Cl)nc2c1CN(C(=O)N1CCc3ccncc31)C2)c1cc(N)cc(C(F)(F)F)c1]

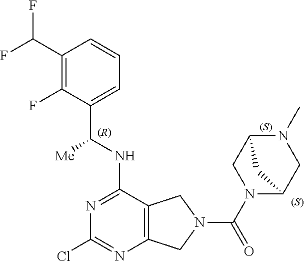

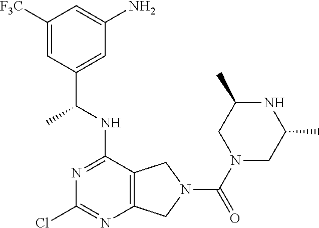 [SMILES: C[C@@H]1CN(C(=O)N2Cc3nc(Cl)nc(N[C@H](C)c4cc(N)cc(C(F)(F)F)c4)c3C2)C[C@@H](C)N1]

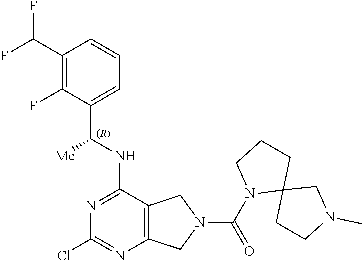

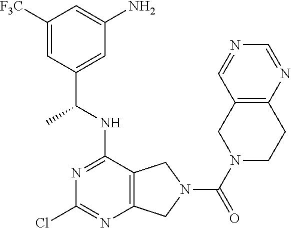 [SMILES: C[C@@H](Nc1nc(Cl)nc2c1CN(C(=O)N1CCc3ncncc3C1)C2)c1cc(N)cc(C(F)(F)F)c1]

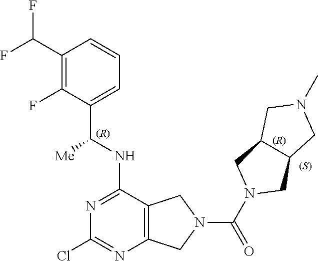

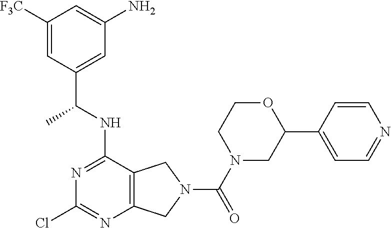 [SMILES: C[C@@H](Nc1nc(Cl)nc2c1CN(C(=O)N1CCOC(c3ccncc3)C1)C2)c1cc(N)cc(C(F)(F)F)c1]

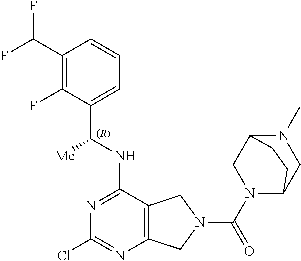

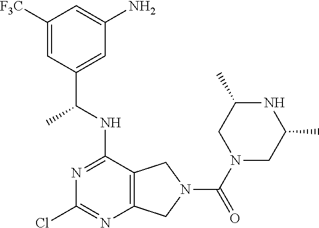 [SMILES: C[C@@H]1CN(C(=O)N2Cc3nc(Cl)nc(N[C@H](C)c4cc(N)cc(C(F)(F)F)c4)c3C2)C[C@H](C)N1]

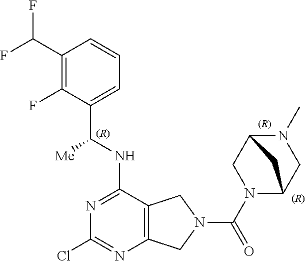

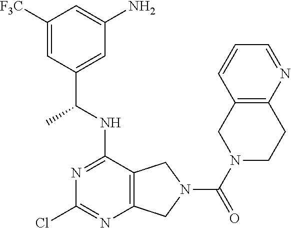 [SMILES: C[C@@H](Nc1nc(Cl)nc2c1CN(C(=O)N1CCc3ncccc3C1)C2)c1cc(N)cc(C(F)(F)F)c1]

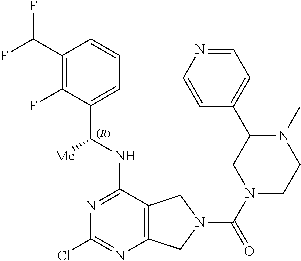

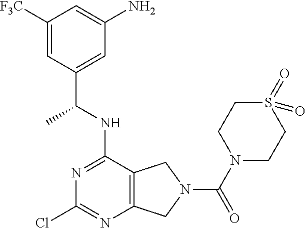 [SMILES: C[C@@H](Nc1nc(Cl)nc2c1CN(C(=O)N1CCS(=O)(=O)CC1)C2)c1cc(N)cc(C(F)(F)F)c1]

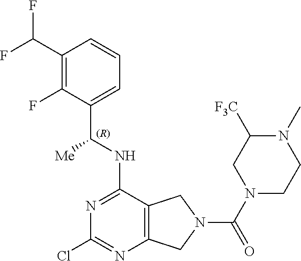

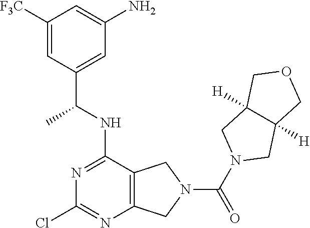 [SMILES: C[C@@H](Nc1nc(Cl)nc2c1CN(C(=O)N1C[C@H]3COC[C@H]3C1)C2)c1cc(N)cc(C(F)(F)F)c1]

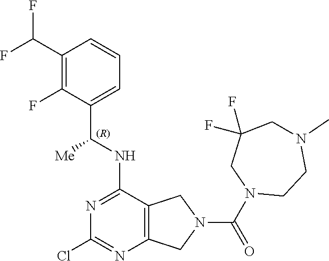

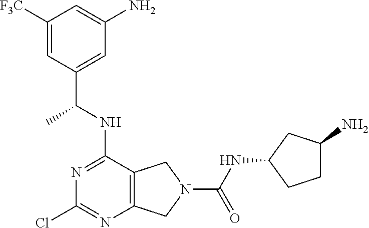 [SMILES: C[C@@H](Nc1nc(Cl)nc2c1CN(C(=O)N[C@H]1CC[C@H](N)C1)C2)c1cc(N)cc(C(F)(F)F)c1]

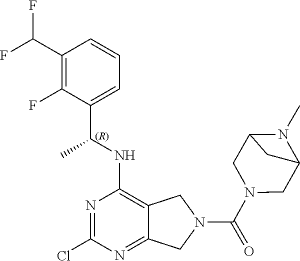

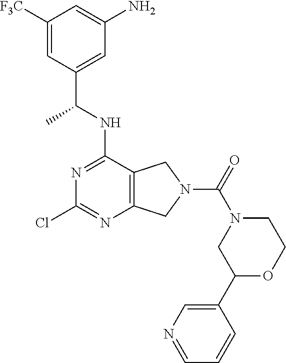 [SMILES: C[C@@H](Nc1nc(Cl)nc2c1CN(C(=O)N1CCOC(c3cccnc3)C1)C2)c1cc(N)cc(C(F)(F)F)c1]

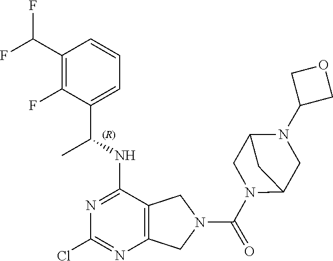

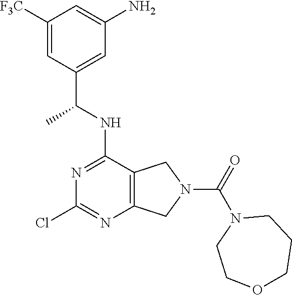 [SMILES: C[C@@H](Nc1nc(Cl)nc2c1CN(C(=O)N1CCCOCC1)C2)c1cc(N)cc(C(F)(F)F)c1]

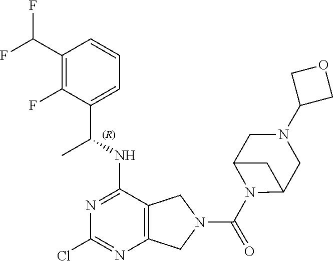

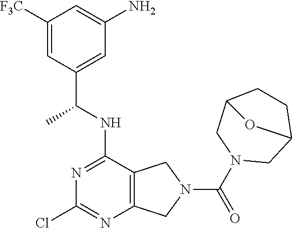 [SMILES: C[C@@H](Nc1nc(Cl)nc2c1CN(C(=O)N1CC3CCC(C1)O3)C2)c1cc(N)cc(C(F)(F)F)c1]

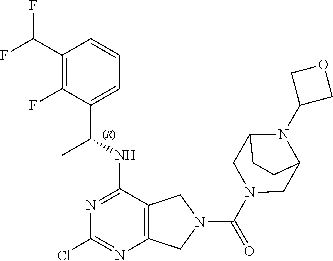

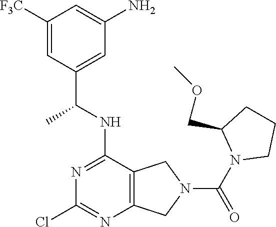 [SMILES: COC[C@H]1CCCN1C(=O)N1Cc2nc(Cl)nc(N[C@H](C)c3cc(N)cc(C(F)(F)F)c3)c2C1]

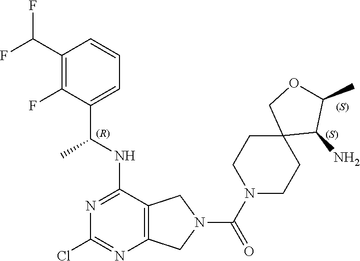

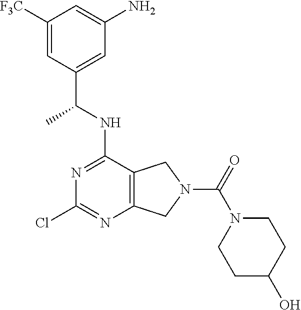 [SMILES: C[C@@H](Nc1nc(Cl)nc2c1CN(C(=O)N1CCC(O)CC1)C2)c1cc(N)cc(C(F)(F)F)c1]

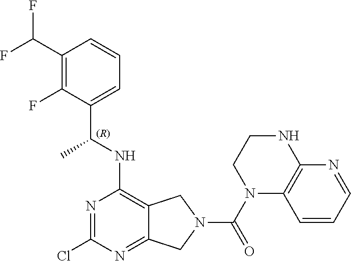

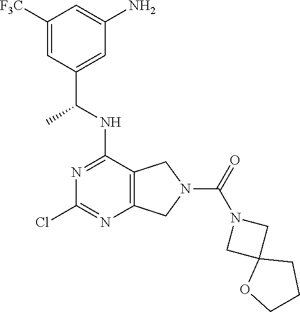 [SMILES: C[C@@H](Nc1nc(Cl)nc2c1CN(C(=O)N1CC3(CCCO3)C1)C2)c1cc(N)cc(C(F)(F)F)c1]

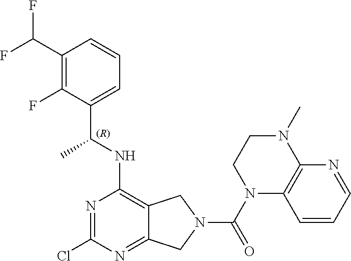

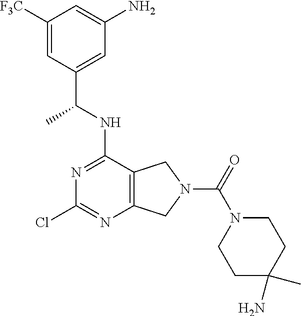 [SMILES: C[C@@H](Nc1nc(Cl)nc2c1CN(C(=O)N1CCC(C)(N)CC1)C2)c1cc(N)cc(C(F)(F)F)c1]

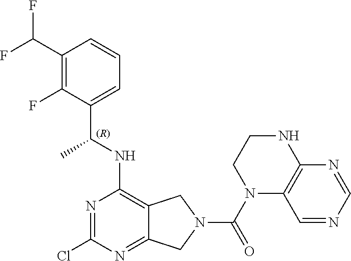

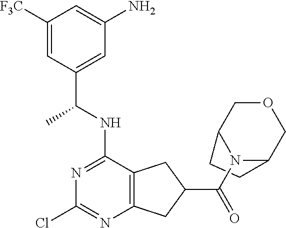 [SMILES: C[C@@H](Nc1nc(Cl)nc2c1CC(C(=O)N1C3CCC1COC3)C2)c1cc(N)cc(C(F)(F)F)c1]

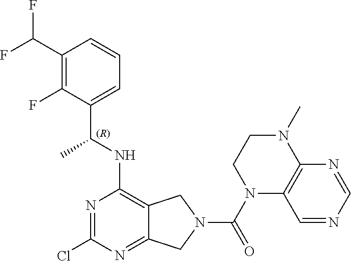

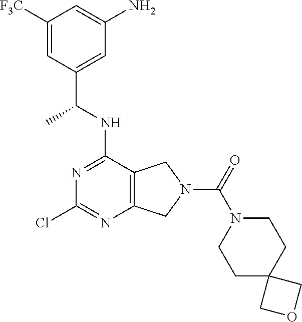 [SMILES: C[C@@H](Nc1nc(Cl)nc2c1CN(C(=O)N1CCC3(CC1)COC3)C2)c1cc(N)cc(C(F)(F)F)c1]

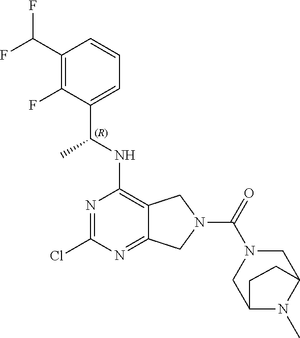

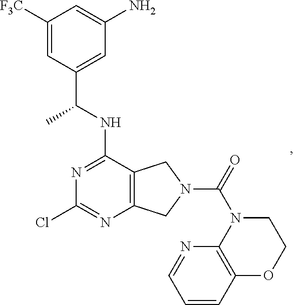 [SMILES: C[C@@H](Nc1nc(Cl)nc2c1CN(C(=O)N1CCOc3cccnc31)C2)c1cc(N)cc(C(F)(F)F)c1]

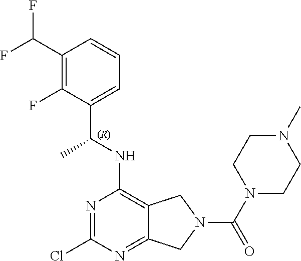

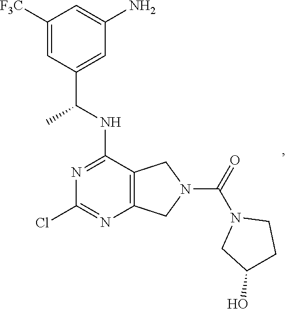 [SMILES: C[C@@H](Nc1nc(Cl)nc2c1CN(C(=O)N1CC[C@H](O)C1)C2)c1cc(N)cc(C(F)(F)F)c1]

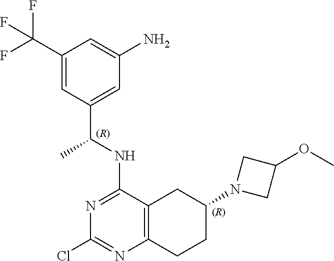

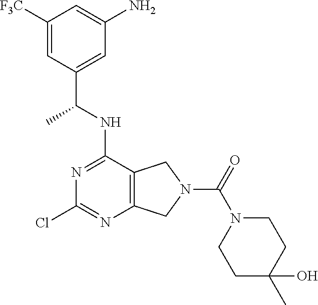 [SMILES: C[C@@H](Nc1nc(Cl)nc2c1CN(C(=O)N1CCC(C)(O)CC1)C2)c1cc(N)cc(C(F)(F)F)c1]

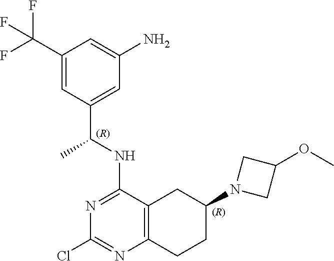

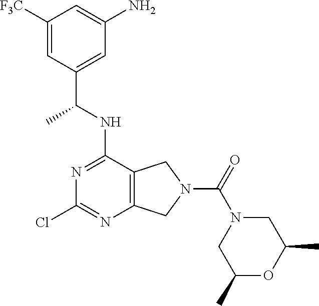 [SMILES: C[C@@H]1CN(C(=O)N2Cc3nc(Cl)nc(N[C@H](C)c4cc(N)cc(C(F)(F)F)c4)c3C2)C[C@H](C)O1]

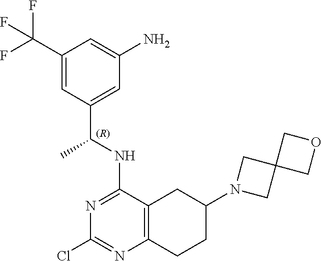

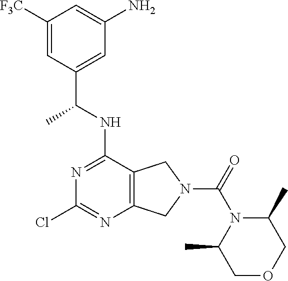 [SMILES: C[C@@H]1COC[C@H](C)N1C(=O)N1Cc2nc(Cl)nc(N[C@H](C)c3cc(N)cc(C(F)(F)F)c3)c2C1]

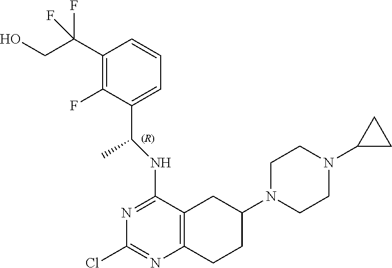

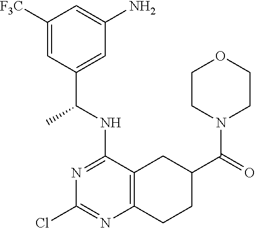 [SMILES: C[C@@H](Nc1nc(Cl)nc2c1CC(C(=O)N1CCOCC1)CC2)c1cc(N)cc(C(F)(F)F)c1]

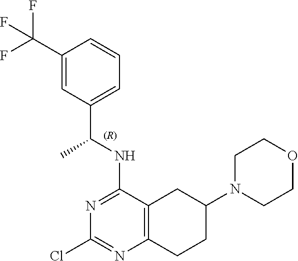

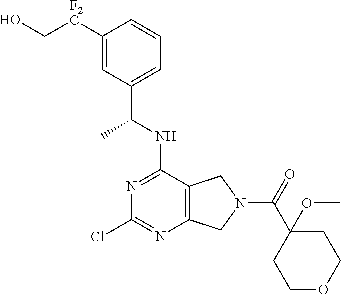 [SMILES: COC1(C(=O)N2Cc3nc(Cl)nc(N[C@H](C)c4cccc(C(F)(F)CO)c4)c3C2)CCOCC1]

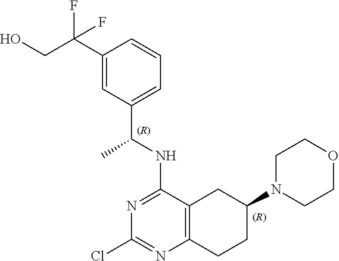

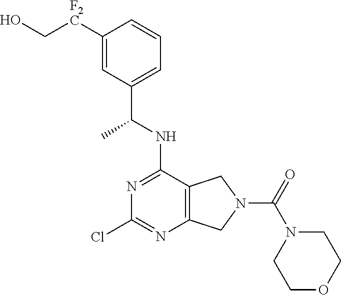 [SMILES: C[C@@H](Nc1nc(Cl)nc2c1CN(C(=O)N1CCOCC1)C2)c1cccc(C(F)(F)CO)c1]

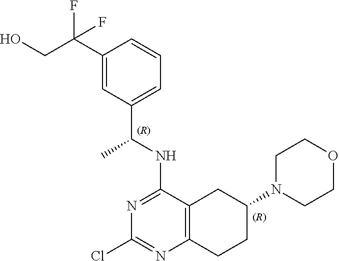

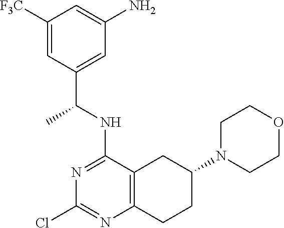 [SMILES: C[C@@H](Nc1nc(Cl)nc2c1C[C@H](N1CCOCC1)CC2)c1cc(N)cc(C(F)(F)F)c1]

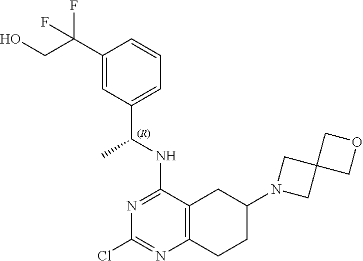

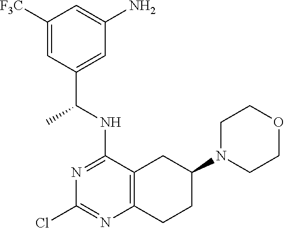 [SMILES: C[C@@H](Nc1nc(Cl)nc2c1C[C@@H](N1CCOCC1)CC2)c1cc(N)cc(C(F)(F)F)c1]

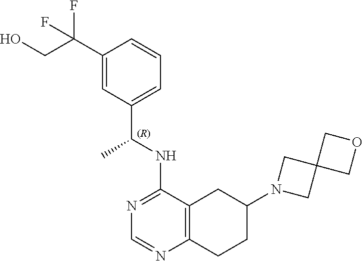

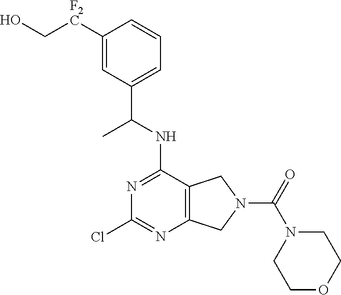 [SMILES: CC(Nc1nc(Cl)nc2c1CN(C(=O)N1CCOCC1)C2)c1cccc(C(F)(F)CO)c1]

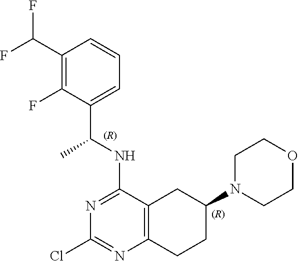

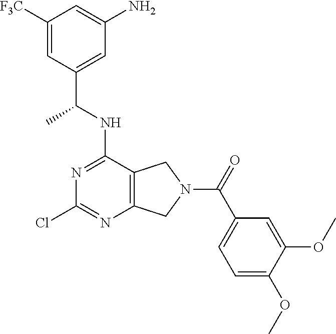 [SMILES: COc1ccc(C(=O)N2Cc3nc(Cl)nc(N[C@H](C)c4cc(N)cc(C(F)(F)F)c4)c3C2)cc1OC]

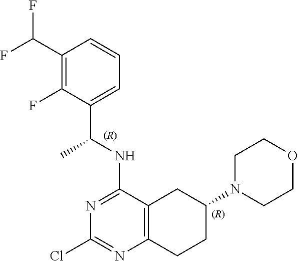

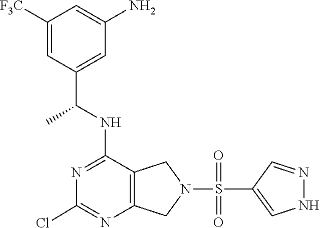 [SMILES: C[C@@H](Nc1nc(Cl)nc2c1CN(S(=O)(=O)c1cn[nH]c1)C2)c1cc(N)cc(C(F)(F)F)c1]

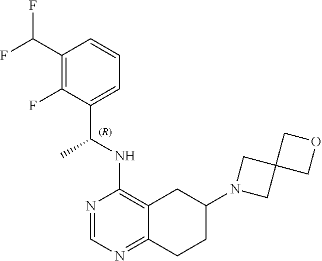

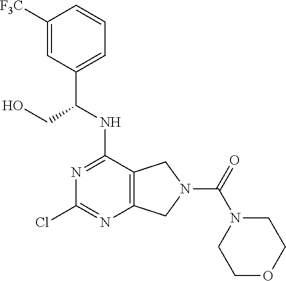 [SMILES: O=C(N1CCOCC1)N1Cc2nc(Cl)nc(N[C@H](CO)c3cccc(C(F)(F)F)c3)c2C1]

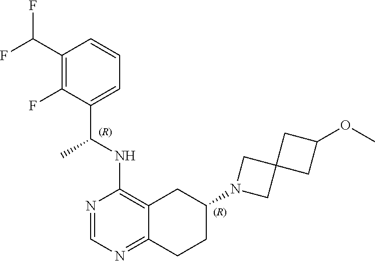

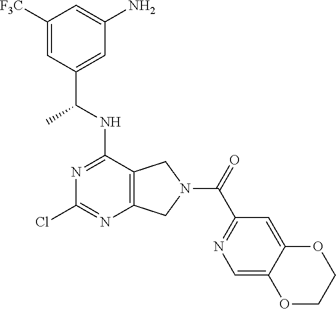 [SMILES: C[C@@H](Nc1nc(Cl)nc2c1CN(C(=O)c1cc3c(cn1)OCCO3)C2)c1cc(N)cc(C(F)(F)F)c1]

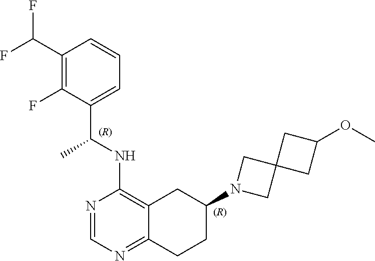

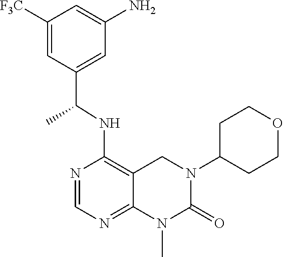 [SMILES: C[C@@H](Nc1ncnc2c1CN(C1CCOCC1)C(=O)N2C)c1cc(N)cc(C(F)(F)F)c1]

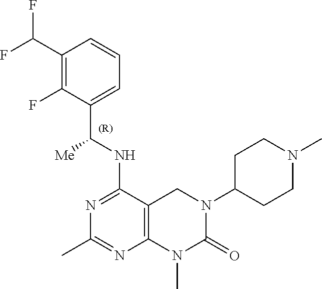

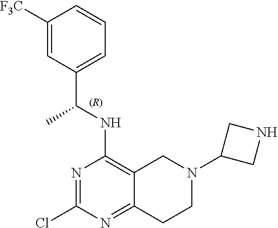

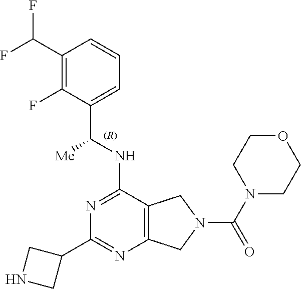

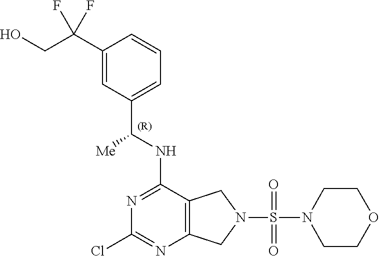

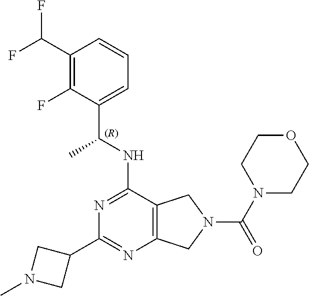

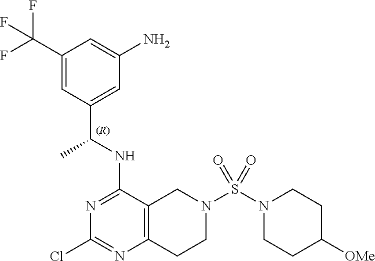

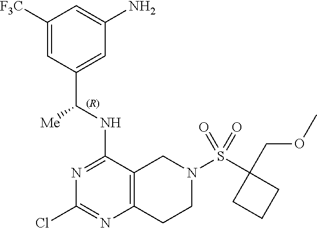

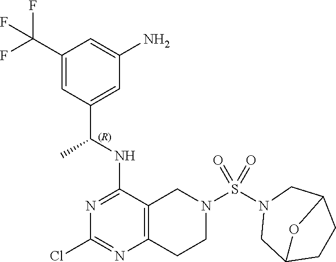

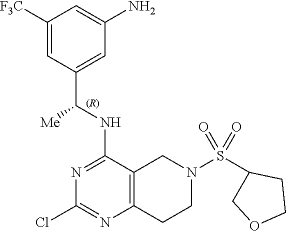

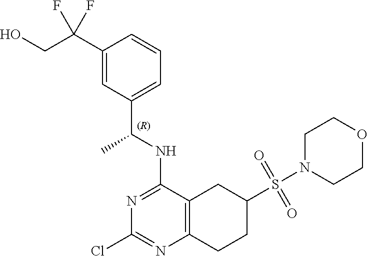

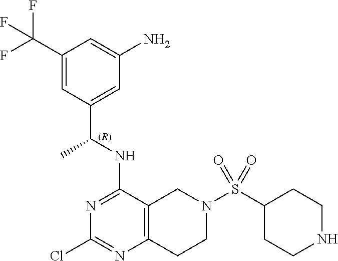

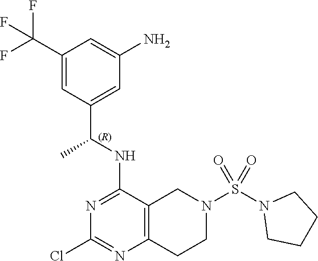

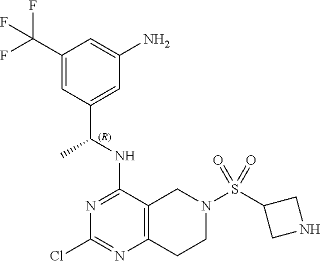

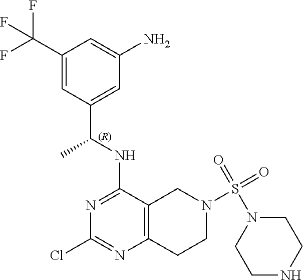

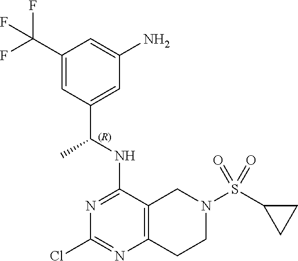

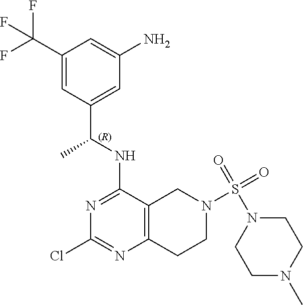

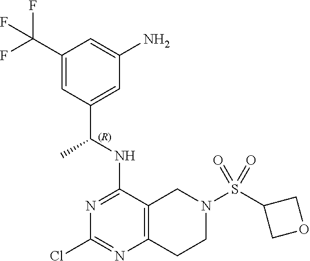

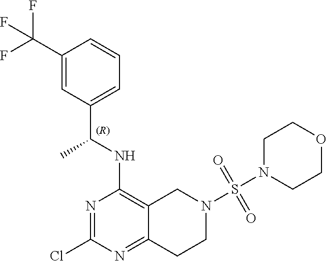

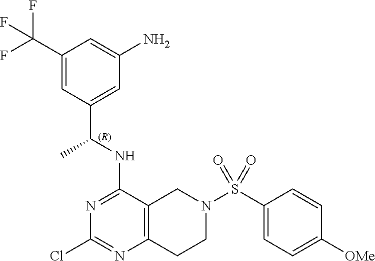

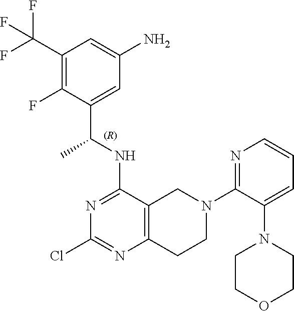

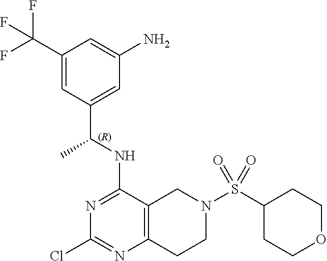

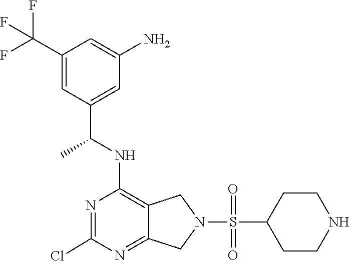 [SMILES: C[C@@H](Nc1nc(Cl)nc2c1CN(S(=O)(=O)C1CCNCC1)C2)c1cc(N)cc(C(F)(F)F)c1]

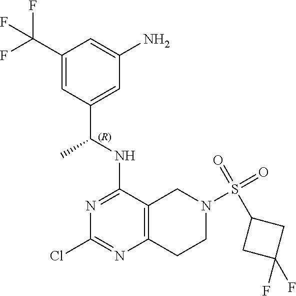

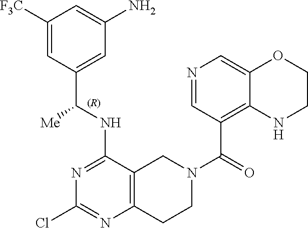

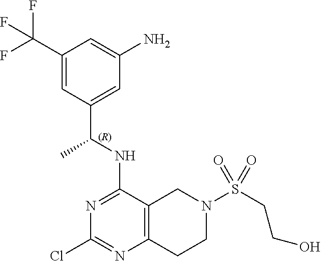

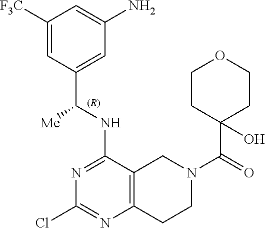

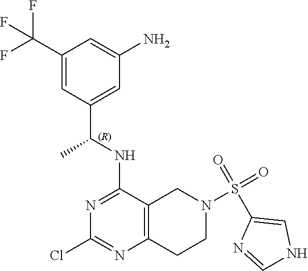

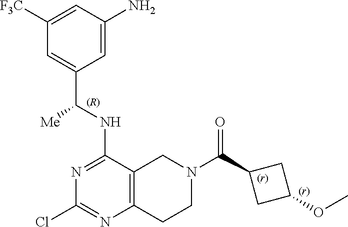

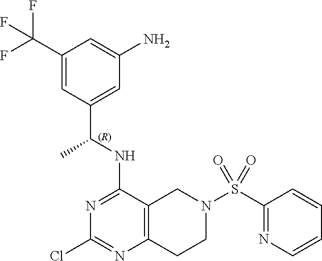

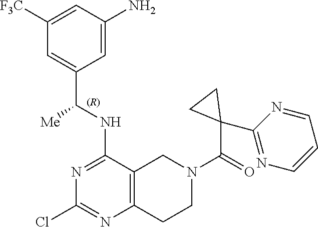

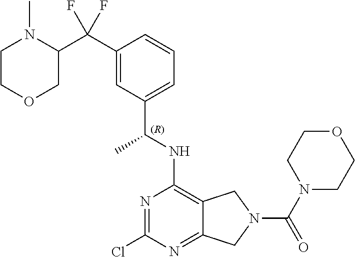

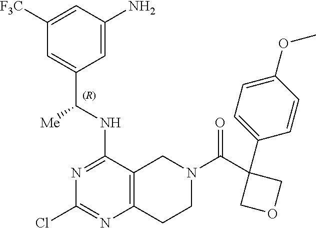

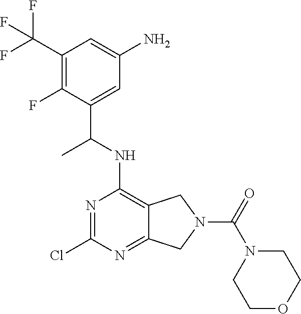 [SMILES: CC(Nc1nc(Cl)nc2c1CN(C(=O)N1CCOCC1)C2)c1cc(N)cc(C(F)(F)F)c1F]

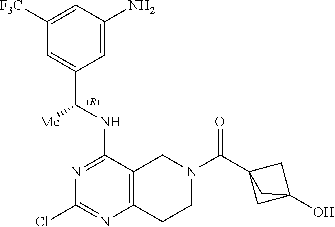

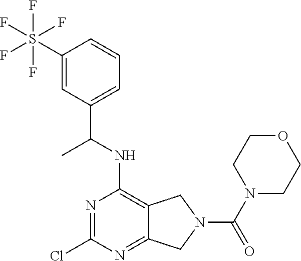 [SMILES: CC(Nc1nc(Cl)nc2c1CN(C(=O)N1CCOCC1)C2)c1cccc(S(F)(F)(F)(F)F)c1]

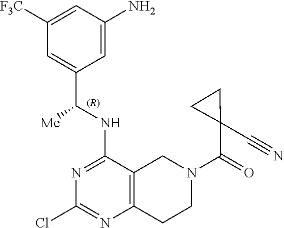

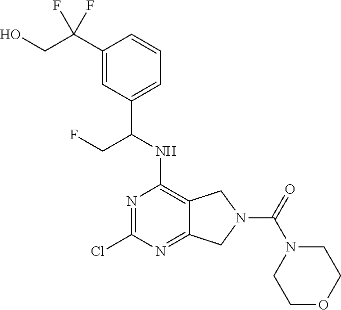 [SMILES: O=C(N1CCOCC1)N1Cc2nc(Cl)nc(NC(CF)c3cccc(C(F)(F)CO)c3)c2C1]

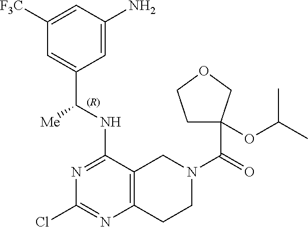

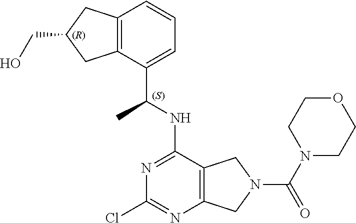

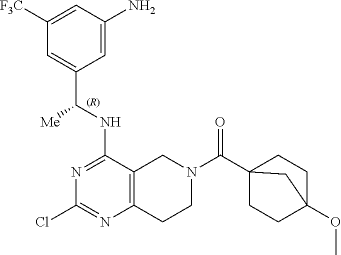

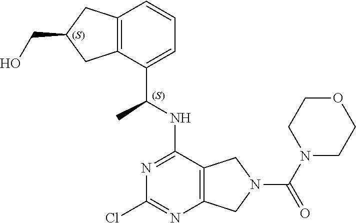

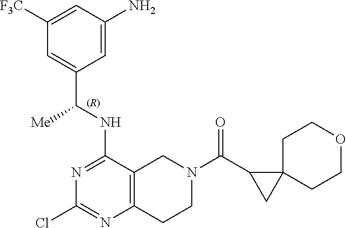

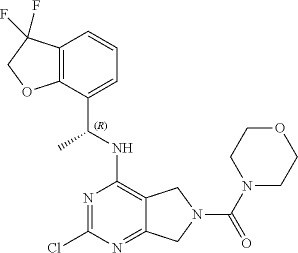

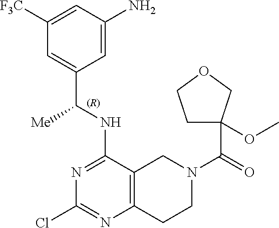

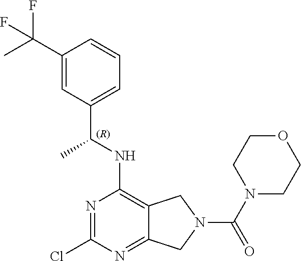

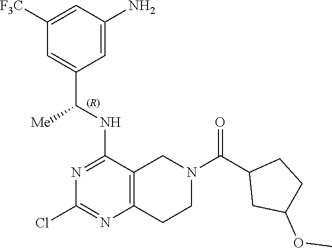

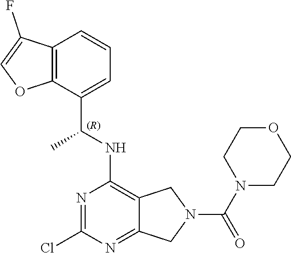

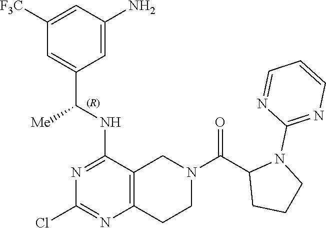

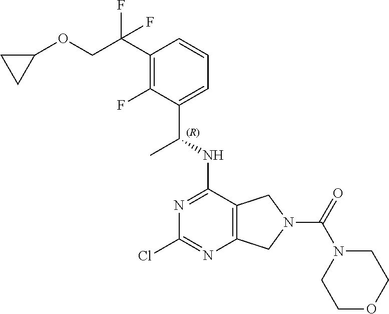

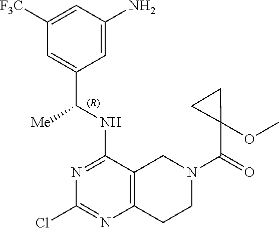

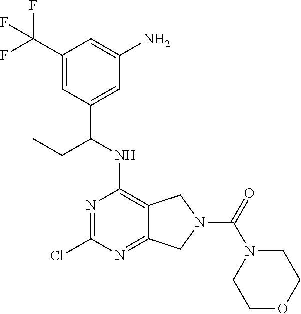 [SMILES: CCC(Nc1nc(Cl)nc2c1CN(C(=O)N1CCOCC1)C2)c1cc(N)cc(C(F)(F)F)c1]

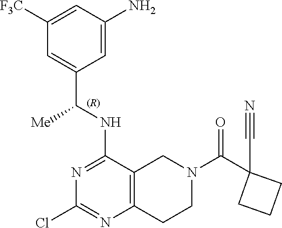

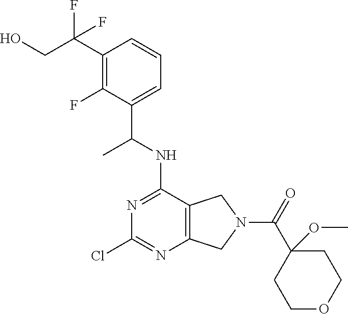 [SMILES: COC1(C(=O)N2Cc3nc(Cl)nc(NC(C)c4cccc(C(F)(F)CO)c4F)c3C2)CCOCC1]

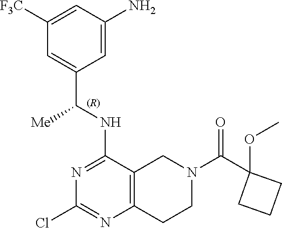

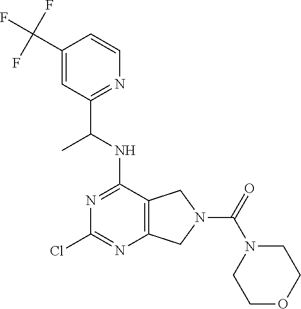 [SMILES: CC(Nc1nc(Cl)nc2c1CN(C(=O)N1CCOCC1)C2)c1cc(C(F)(F)F)ccn1]

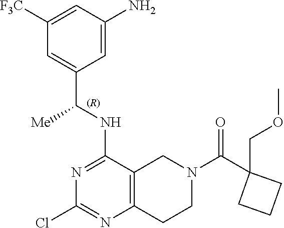

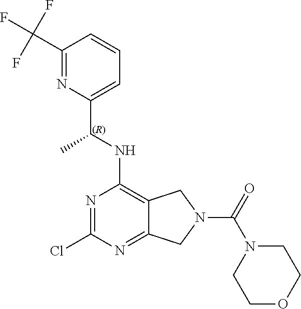

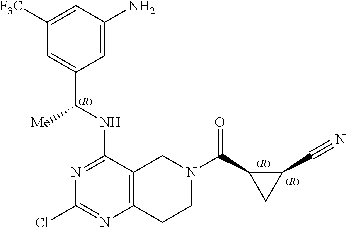

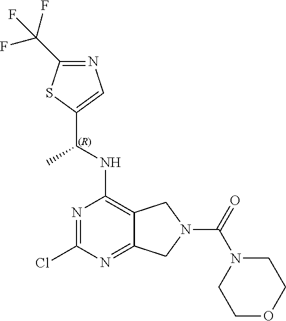

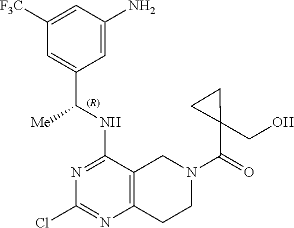

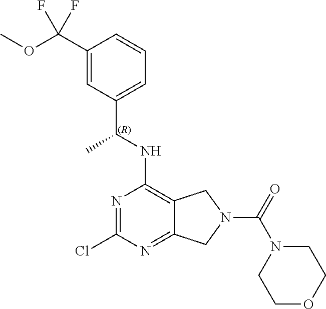

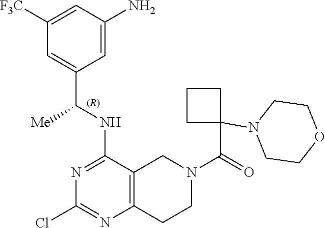

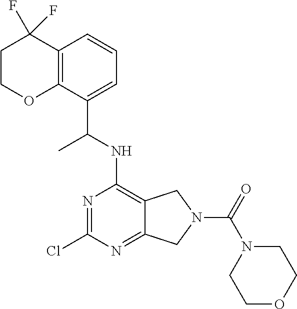 [SMILES: CC(Nc1nc(Cl)nc2c1CN(C(=O)N1CCOCC1)C2)c1cccc2c1OCCC2(F)F]

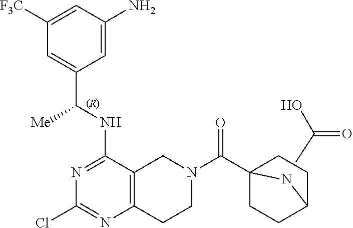

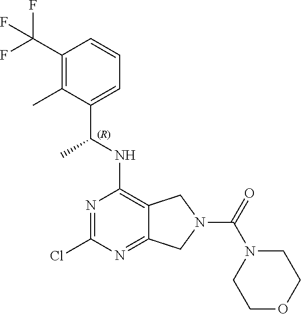

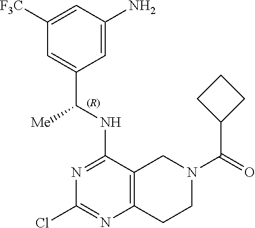

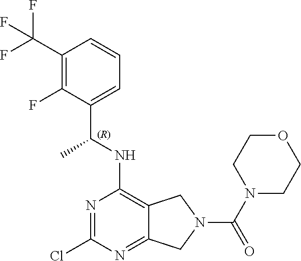

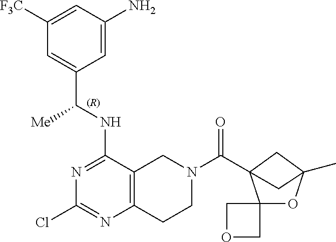

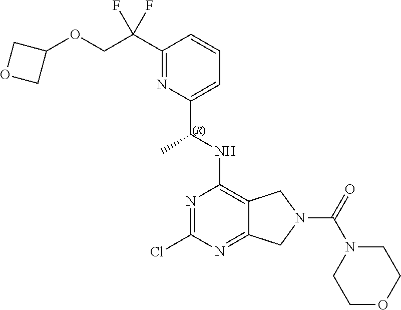

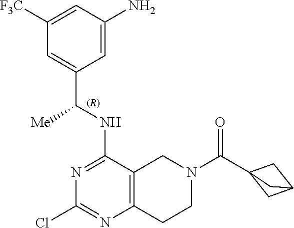

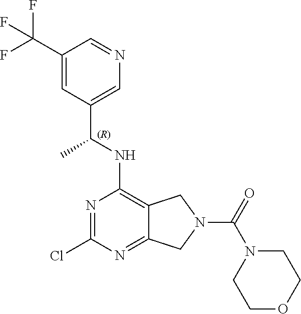

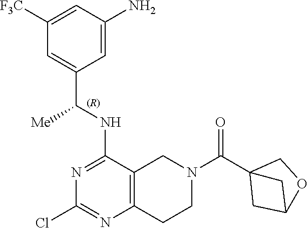

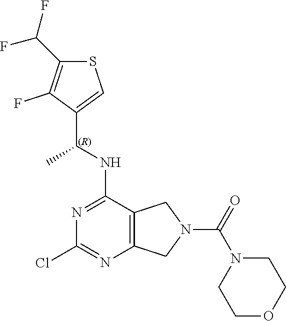

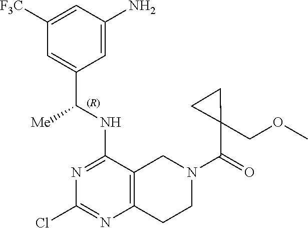

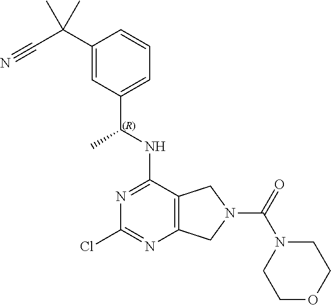

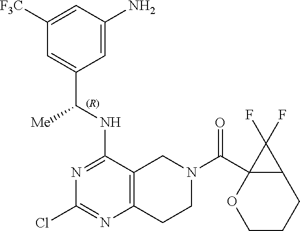

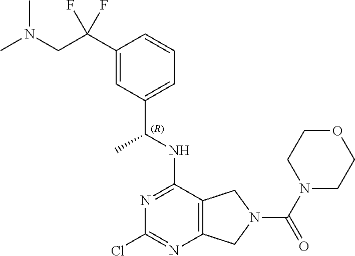

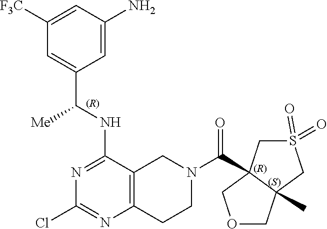

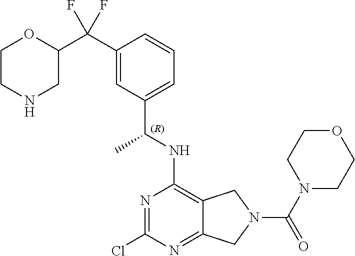

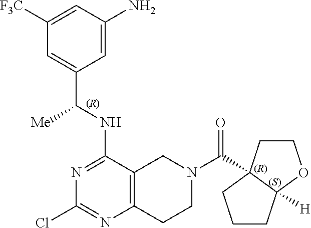

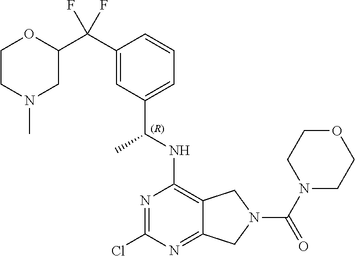

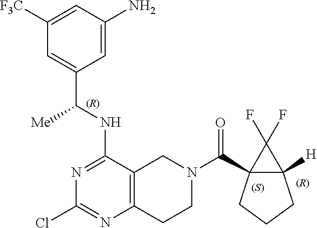

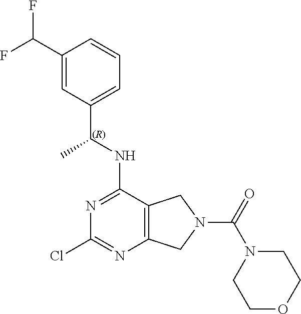

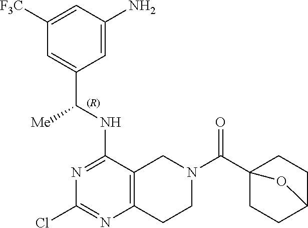

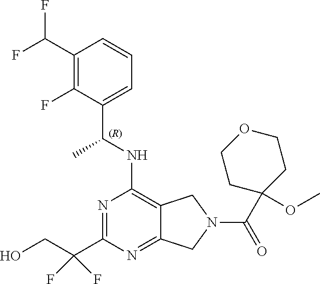

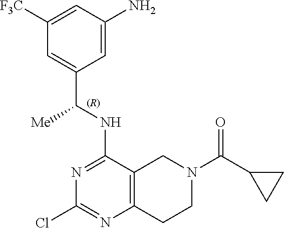

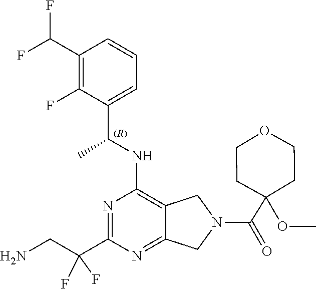

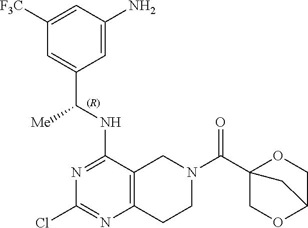

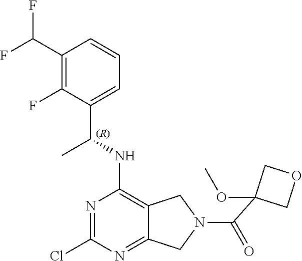

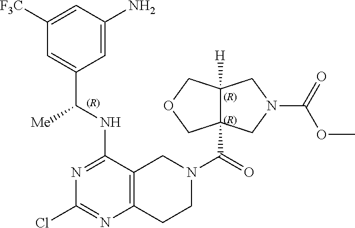

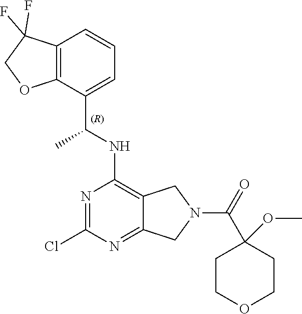

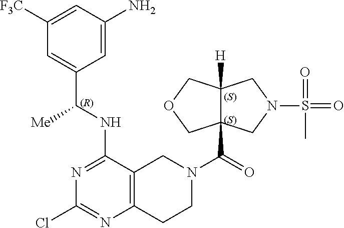

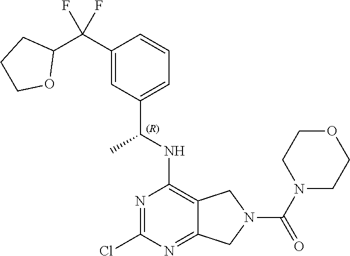

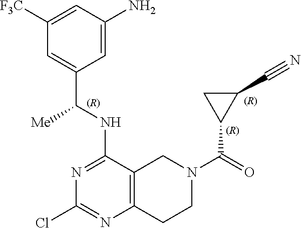

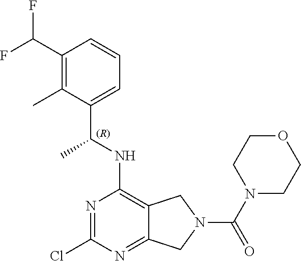

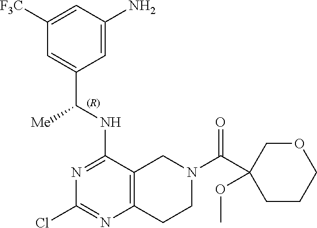

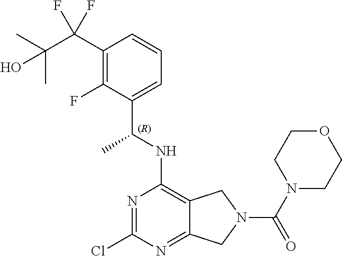

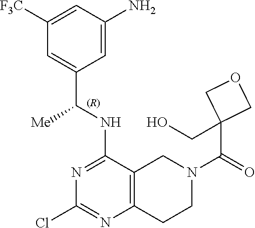

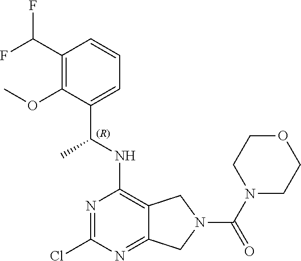

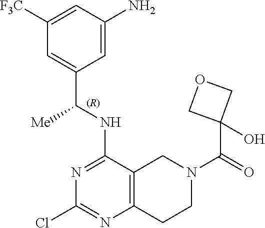

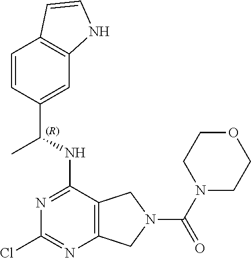

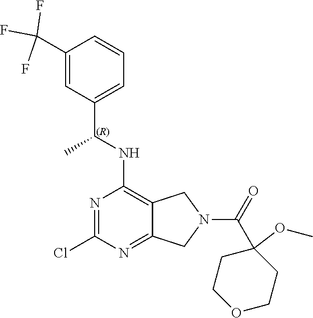

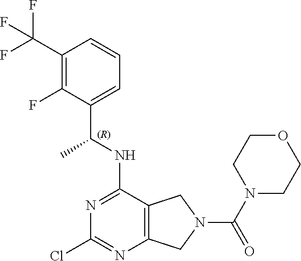

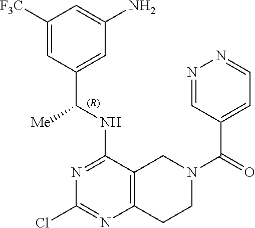

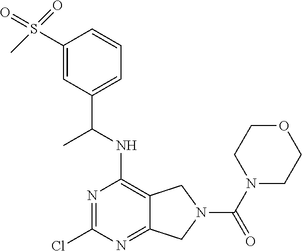 [SMILES: CC(Nc1nc(Cl)nc2c1CN(C(=O)N1CCOCC1)C2)c1cccc(S(C)(=O)=O)c1]

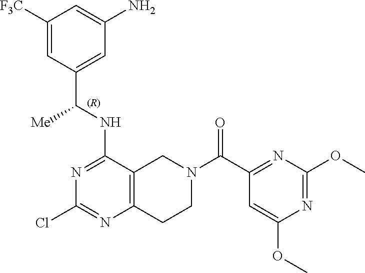

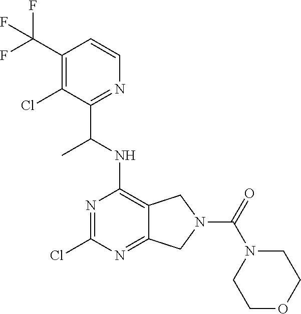 [SMILES: CC(Nc1nc(Cl)nc2c1CN(C(=O)N1CCOCC1)C2)c1nccc(C(F)(F)F)c1Cl]

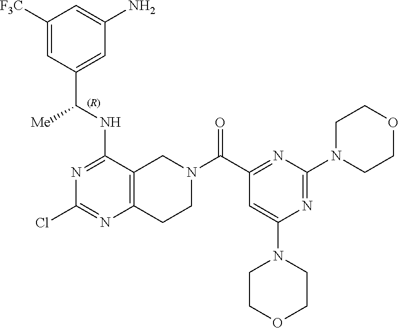

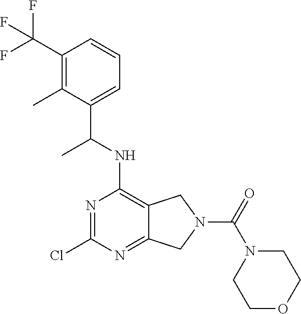 [SMILES: Cc1c(C(C)Nc2nc(Cl)nc3c2CN(C(=O)N2CCOCC2)C3)cccc1C(F)(F)F]

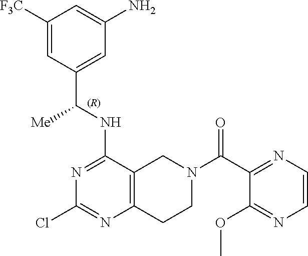

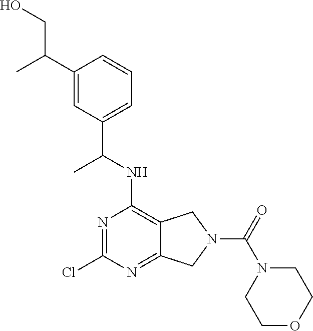 [SMILES: CC(CO)c1cccc(C(C)Nc2nc(Cl)nc3c2CN(C(=O)N2CCOCC2)C3)c1]

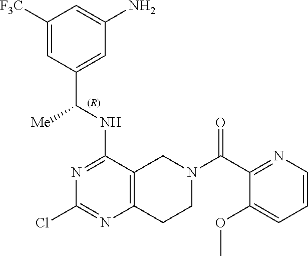

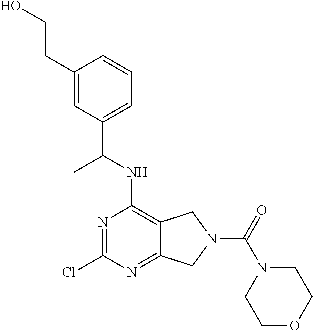 [SMILES: CC(Nc1nc(Cl)nc2c1CN(C(=O)N1CCOCC1)C2)c1cccc(CCO)c1]

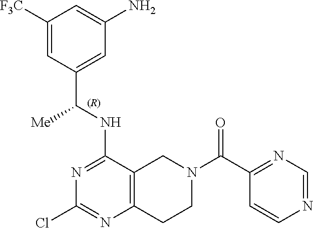

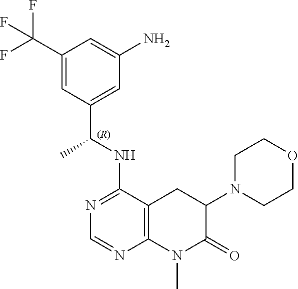

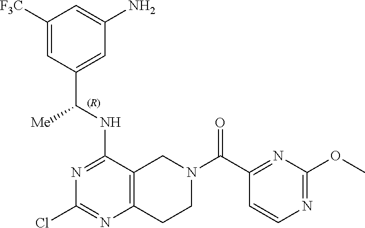

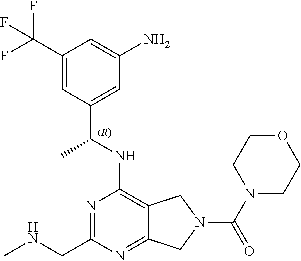

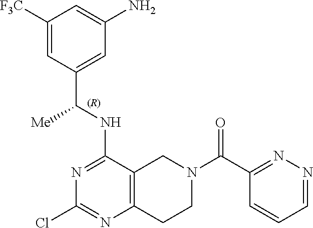

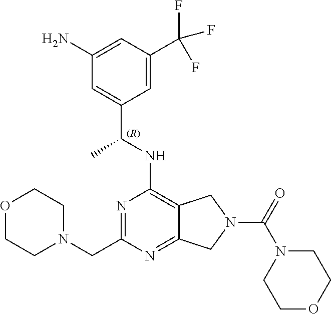

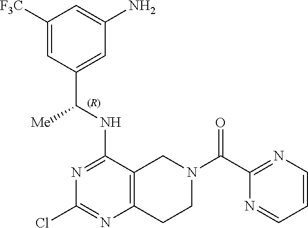

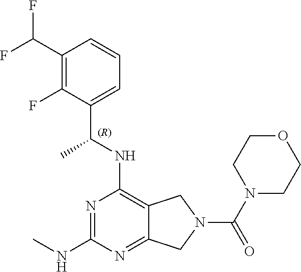

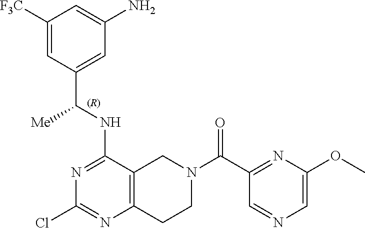

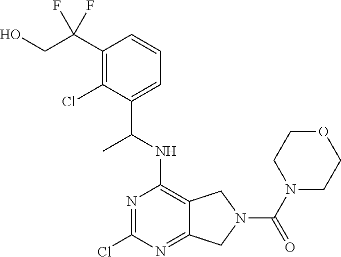 [SMILES: CC(Nc1nc(Cl)nc2c1CN(C(=O)N1CCOCC1)C2)c1cccc(C(F)(F)CO)c1Cl]

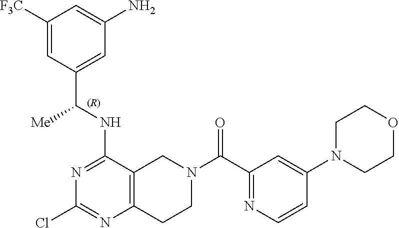

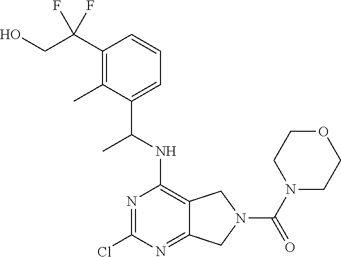 [SMILES: Cc1c(C(C)Nc2nc(Cl)nc3c2CN(C(=O)N2CCOCC2)C3)cccc1C(F)(F)CO]

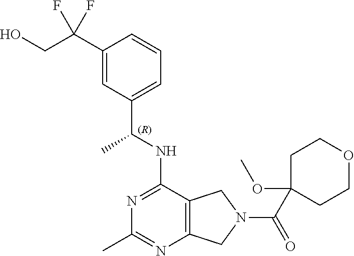

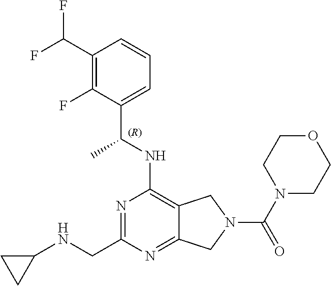

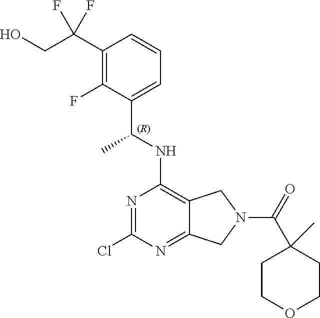

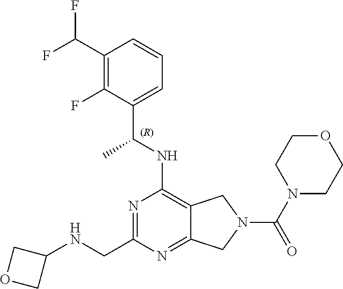

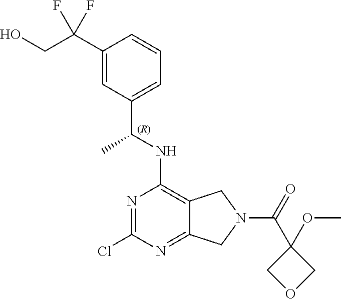

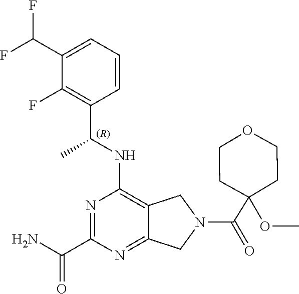

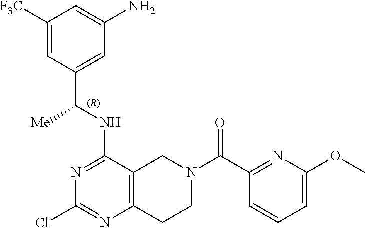

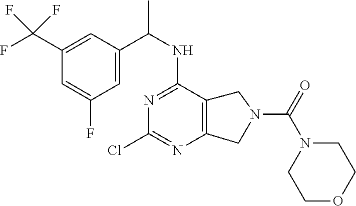 [SMILES: CC(Nc1nc(Cl)nc2c1CN(C(=O)N1CCOCC1)C2)c1cc(F)cc(C(F)(F)F)c1]

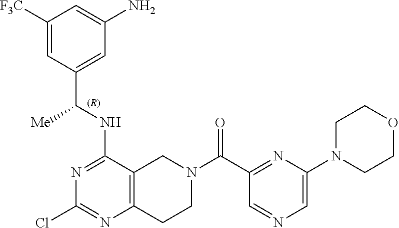

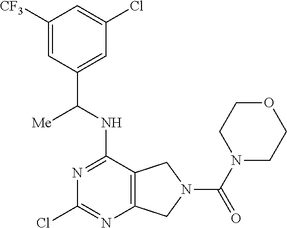 [SMILES: CC(Nc1nc(Cl)nc2c1CN(C(=O)N1CCOCC1)C2)c1cc(Cl)cc(C(F)(F)F)c1]

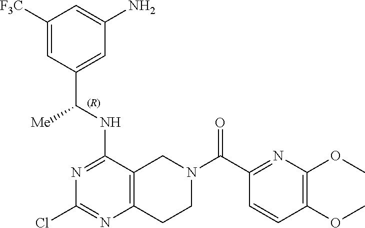

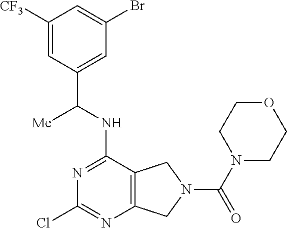 [SMILES: CC(Nc1nc(Cl)nc2c1CN(C(=O)N1CCOCC1)C2)c1cc(Br)cc(C(F)(F)F)c1]

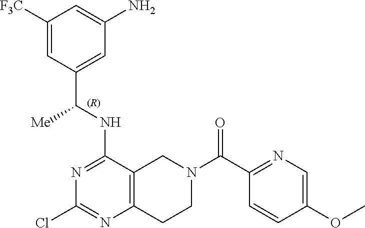

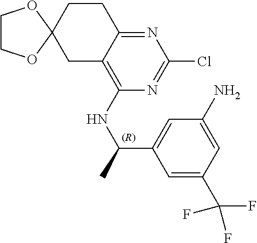

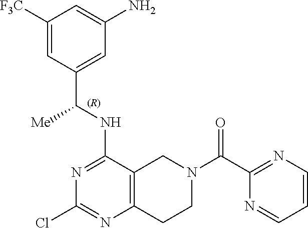

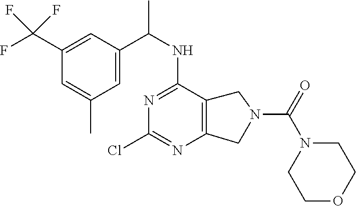 [SMILES: Cc1cc(C(C)Nc2nc(Cl)nc3c2CN(C(=O)N2CCOCC2)C3)cc(C(F)(F)F)c1]

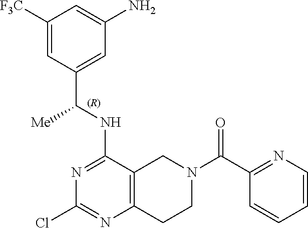

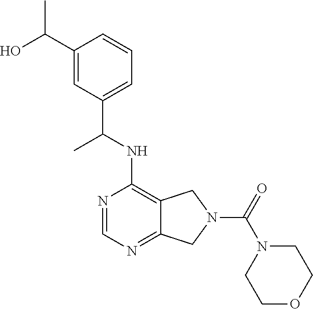 [SMILES: CC(O)c1cccc(C(C)Nc2ncnc3c2CN(C(=O)N2CCOCC2)C3)c1]

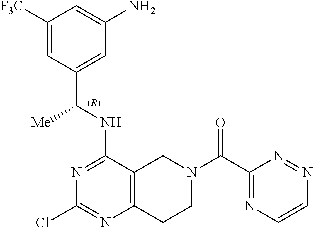

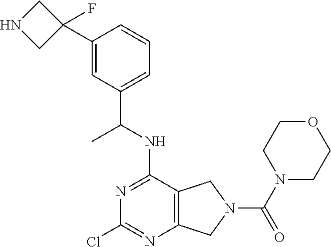 [SMILES: CC(Nc1nc(Cl)nc2c1CN(C(=O)N1CCOCC1)C2)c1cccc(C2(F)CNC2)c1]

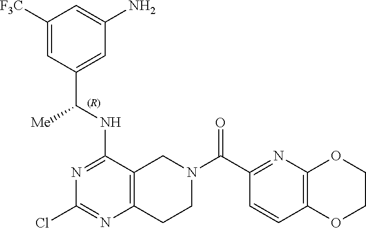

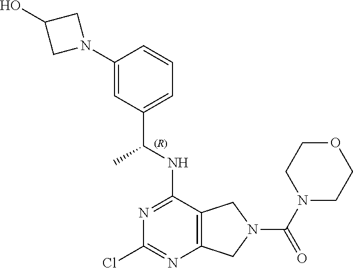

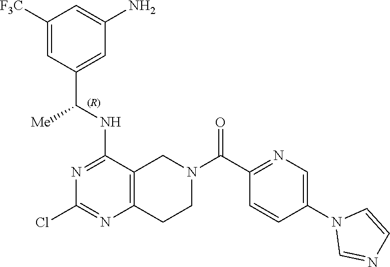

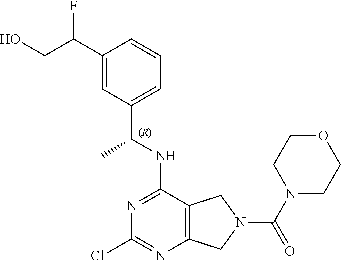

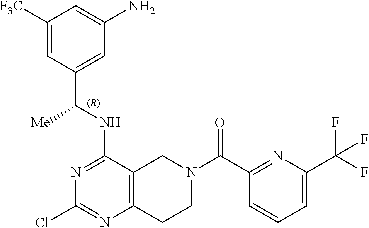

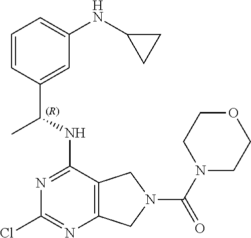

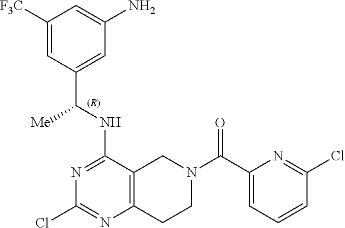

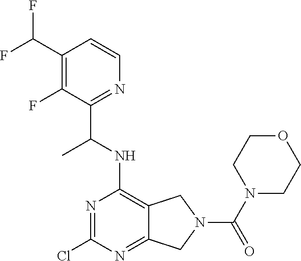 [SMILES: CC(Nc1nc(Cl)nc2c1CN(C(=O)N1CCOCC1)C2)c1nccc(C(F)F)c1F]

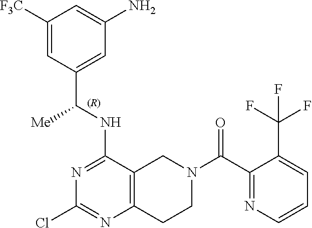

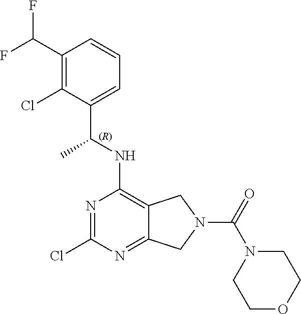

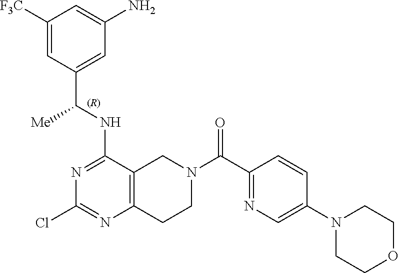

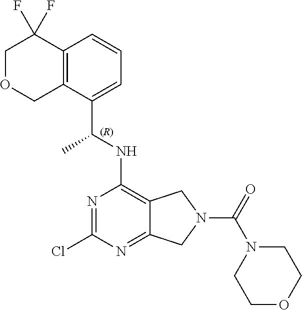

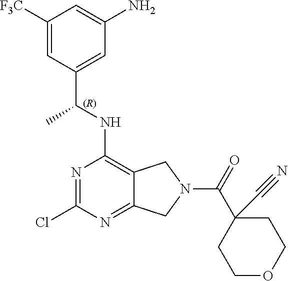

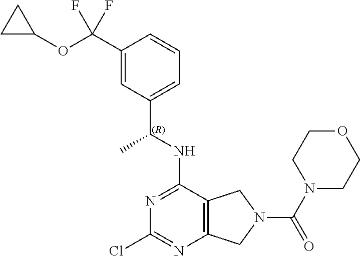

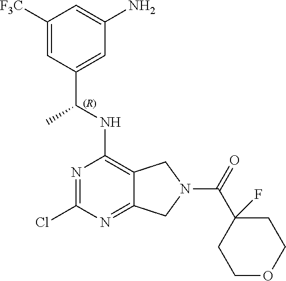

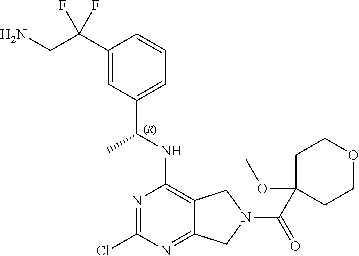

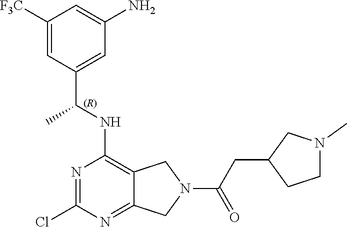

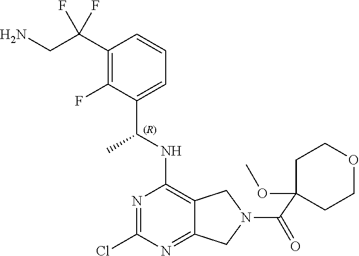

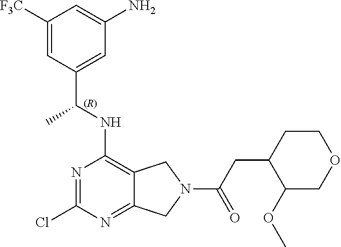

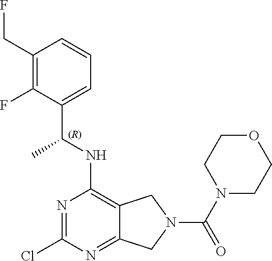

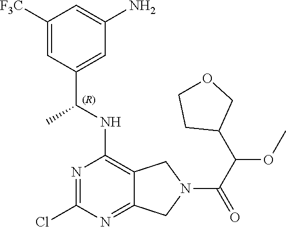

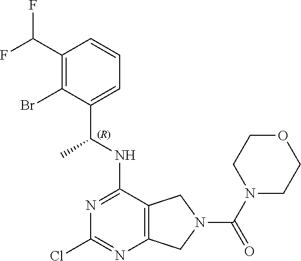

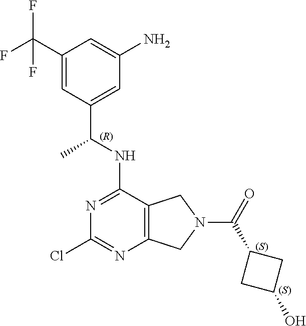

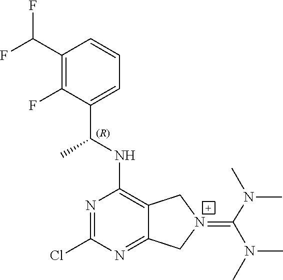

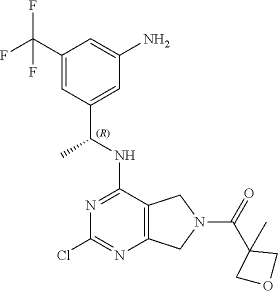

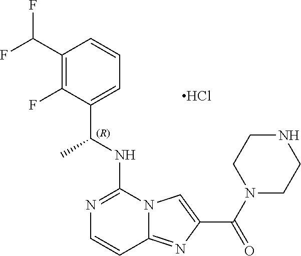

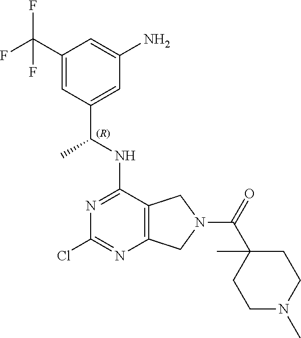

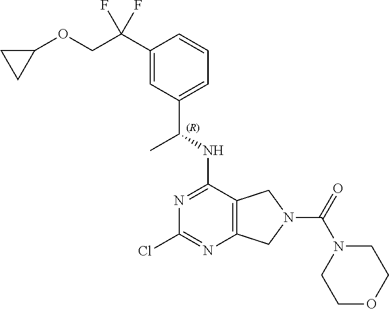

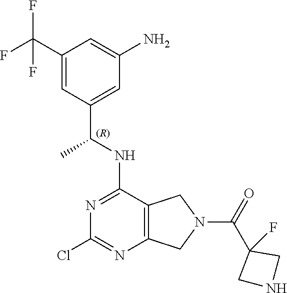

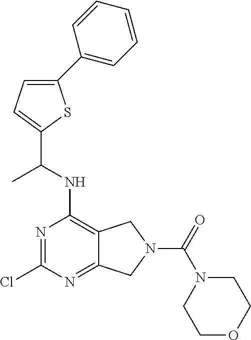 [SMILES: CC(Nc1nc(Cl)nc2c1CN(C(=O)N1CCOCC1)C2)c1ccc(-c2ccccc2)s1]

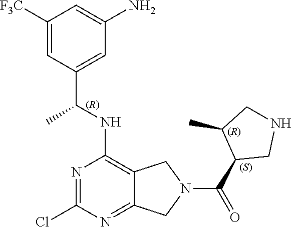

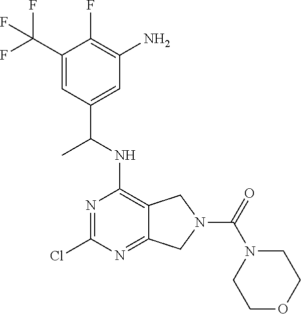 [SMILES: CC(Nc1nc(Cl)nc2c1CN(C(=O)N1CCOCC1)C2)c1cc(N)c(F)c(C(F)(F)F)c1]

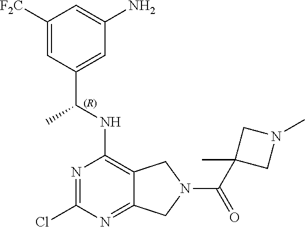

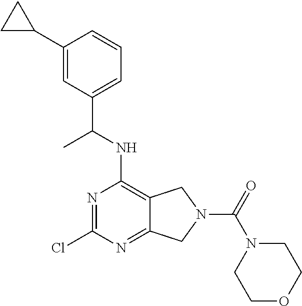 [SMILES: CC(Nc1nc(Cl)nc2c1CN(C(=O)N1CCOCC1)C2)c1cccc(C2CC2)c1]

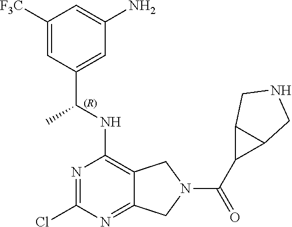

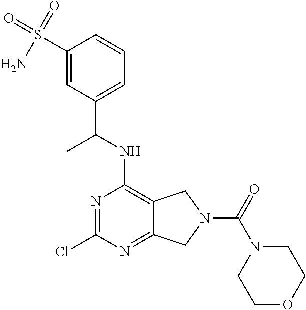 [SMILES: CC(Nc1nc(Cl)nc2c1CN(C(=O)N1CCOCC1)C2)c1cccc(S(N)(=O)=O)c1]

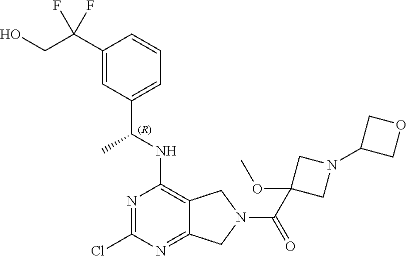

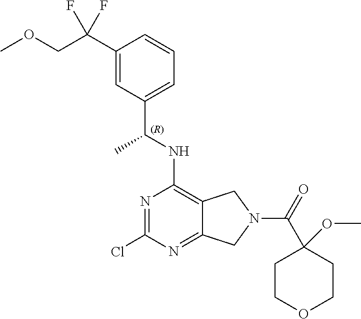

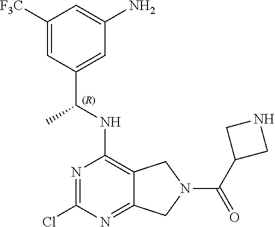

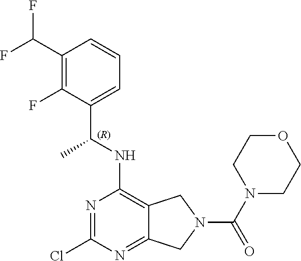

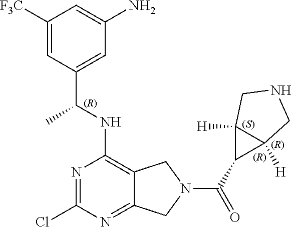

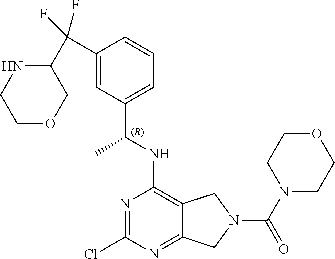

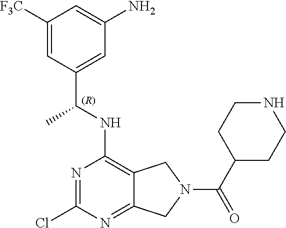

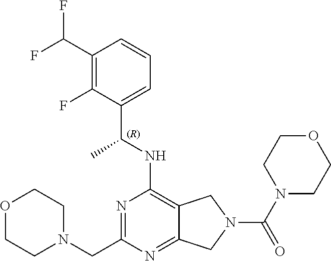

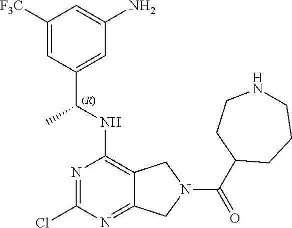

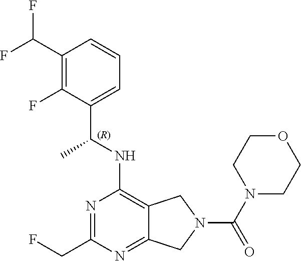

In some embodiments, the SOS1 inhibitor is a compound having the structure of Formula (48-I),

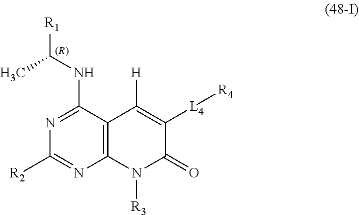

or a pharmaceutically acceptable salt, solvate, isomer (e.g., stereoisomer), prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, C₁₋₆ alkyl, halogen, —NHR₂ₐ, —OR^{2a}, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl;
R³ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, and —CN;
L₄ is selected from the group consisting of bond, —C(O)—, —C(O)O—, —C(O)NH(CH₂)ₒ—, —NH—, —S—, —S(O)₂—,

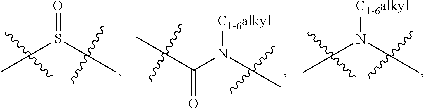

(CH₂)ₚ—, and —O—; wherein o is 0, 1, or 2; and wherein p is a number from 1 to 6; and
R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO) R₄ₐ, —C(O)NR_{4b}R_{4c}, —NR_{4b}C(O)R_{4c}, —CN, ═NR₄ₐ, —NR_{4b}R_{4c}, -SO₂R⁴a, 3-6 membered cycloalkyl optionally substituted with R⁴a, 3-7 membered heterocyclyl optionally substituted with R₄ₐ, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂),OCH₃, or —(CH₂),OH, wherein r is 1, 2, or 3;
   wherein each R_{4b} is independently H, C₁₋₆ alkyl; and
   wherein each R_{4c} is independently H or C₁₋₆ alkyl.

In some embodiments, the SOS1 inhibitor is a compound having the structure of Formula (48-II),

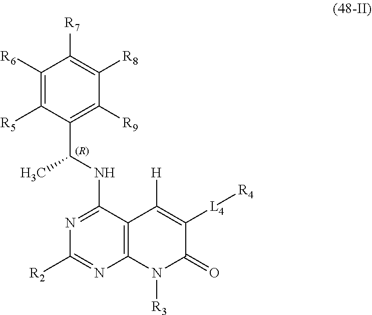

or a pharmaceutically acceptable salt, solvate, isomer (e.g., stereoisomer), prodrug, or tautomer thereof, wherein R₂, R₃, L₄, and R₄ are as defined in Formula (48-I);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with-OH, C₁₋₆ alkyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound having the structure of Formula (48-II),

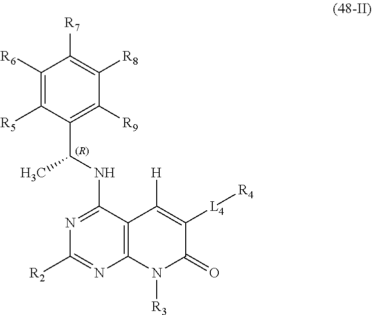

or a pharmaceutically acceptable salt or a stereoisomer thereof, wherein:
R₂ is H;
R₃ is selected from the group consisting of H and C₁₋₃ alkyl;
L₄ is a bond;
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, —O, halogen, —C(O)R₄ₐ, —C(OO) R₄ₐ, —C(O)NR_{4b}R_{4c}, —NR_{4b}C(O)R_{4c}, —CN,═NR₄ₐ, —NR_{4b}R_{4c}, —SO₂R₄ₐ, 3-6 membered cycloalkyl optionally substituted with R₄ₐ, 3-7 membered heterocyclyl optionally substituted with R⁴a, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂),OCH₃, or —(CH₂),OH, wherein r is 1, 2, or 3;
   wherein each R_{4b} is independently H, C₁₋₆ alkyl;
   wherein each R_{4c} is independently H or C₁₋₆ alkyl;
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R¹², —SR₁₀, —S(O)₂NR₁₁R₁₂, —S(O)₂R¹⁰, -NR¹⁰S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with-OH, C₁₋₆ alkyl optionally substituted with —R₁₀, halogen, —NO₂, —O, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl;
R₁₀, R₁₁, and R₁₂ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound selected from the group consisting of the compounds in the following table, or a pharmaceutically acceptable salt, solvate, isomer (e.g., stereoisomer), prodrug, or tautomer thereof:

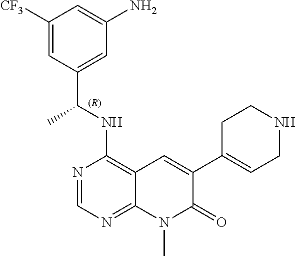

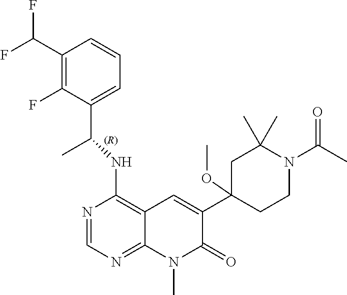

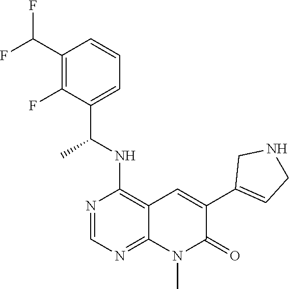 [SMILES: C[C@@H](Nc1ncnc2c1cc(C1=CCNC1)c(=O)n2C)c1cccc(C(F)F)c1F]

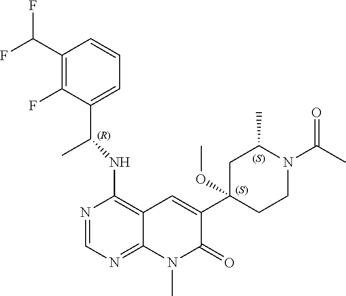

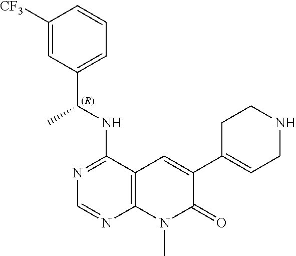

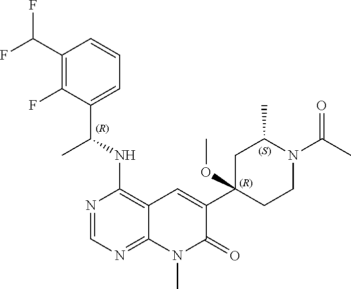

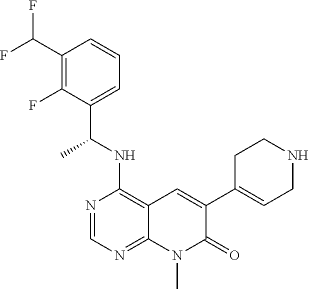 [SMILES: C[C@@H](Nc1ncnc2c1cc(C1=CCNCC1)c(=O)n2C)c1cccc(C(F)F)c1F]

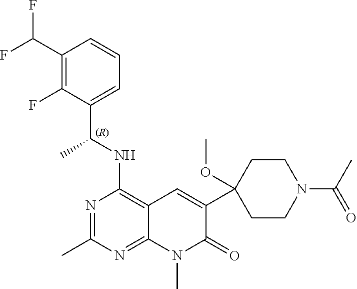

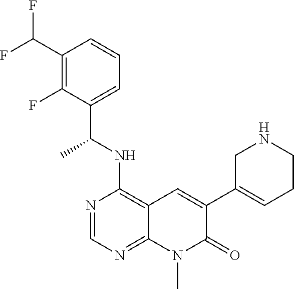 [SMILES: C[C@@H](Nc1ncnc2c1cc(C1=CCCNC1)c(=O)n2C)c1cccc(C(F)F)c1F]

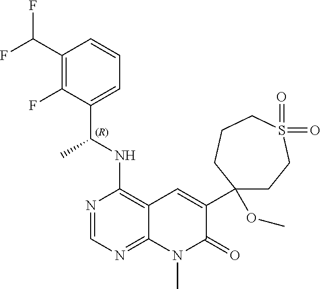

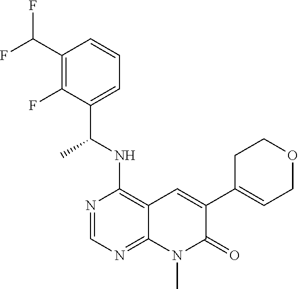 [SMILES: C[C@@H](Nc1ncnc2c1cc(C1=CCOCC1)c(=O)n2C)c1cccc(C(F)F)c1F]

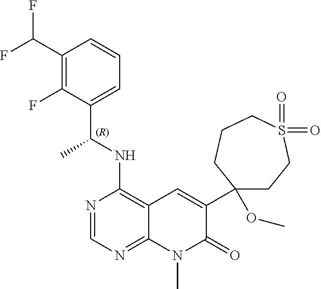

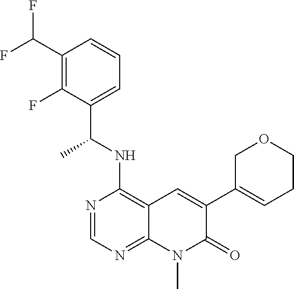 [SMILES: C[C@@H](Nc1ncnc2c1cc(C1=CCCOC1)c(=O)n2C)c1cccc(C(F)F)c1F]

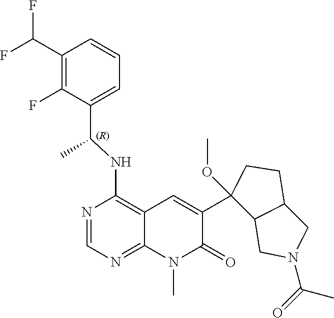

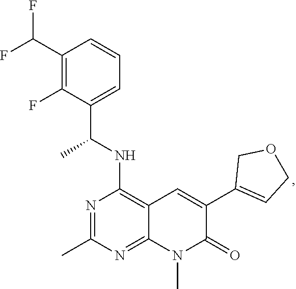 [SMILES: Cc1nc(N[C@H](C)c2cccc(C(F)F)c2F)c2cc(C3=CCOC3)c(=O)n(C)c2n1]

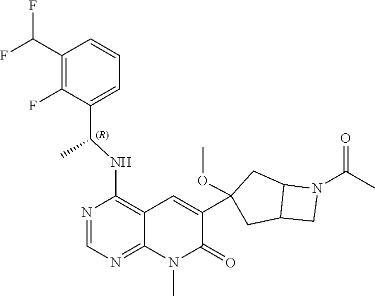

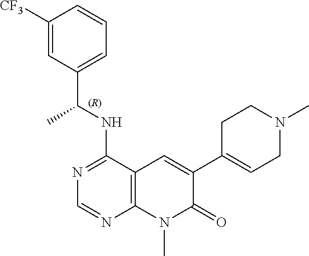

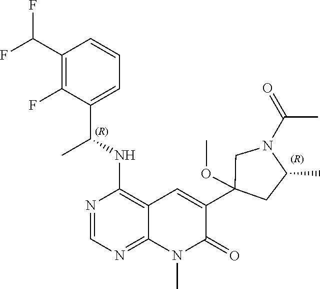

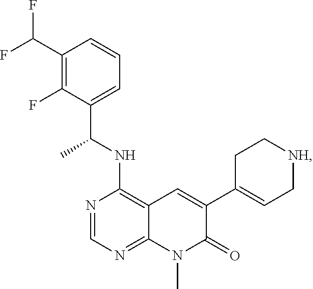 [SMILES: C[C@@H](Nc1ncnc2c1cc(C1=CCNCC1)c(=O)n2C)c1cccc(C(F)F)c1F]

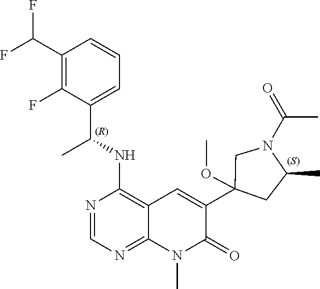

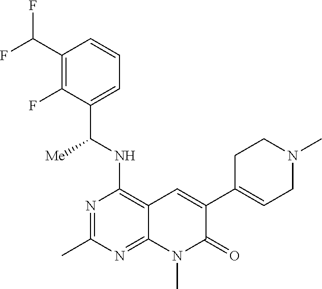 [SMILES: Cc1nc(N[C@H](C)c2cccc(C(F)F)c2F)c2cc(C3=CCN(C)CC3)c(=O)n(C)c2n1]

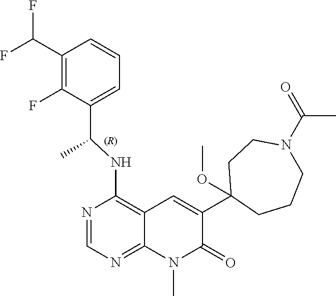

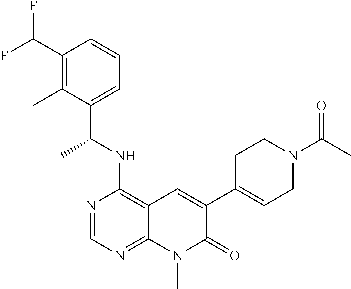 [SMILES: CC(=O)N1CC=C(c2cc3c(N[C@H](C)c4cccc(C(F)F)c4C)ncnc3n(C)c2=O)CC1]

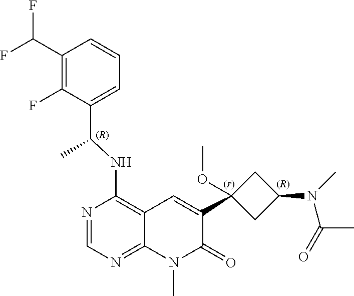

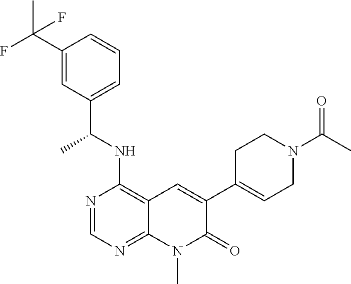 [SMILES: CC(=O)N1CC=C(c2cc3c(N[C@H](C)c4cccc(C(C)(F)F)c4)ncnc3n(C)c2=O)CC1]

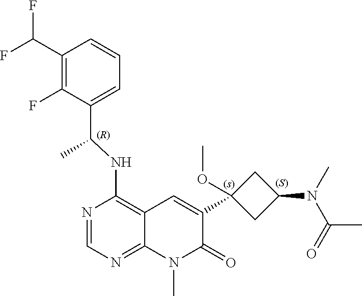

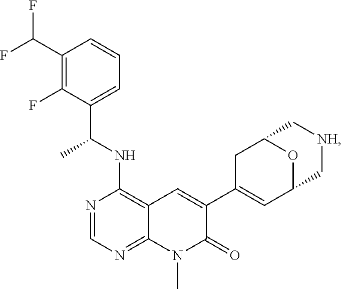 [SMILES: C[C@@H](Nc1ncnc2c1cc(C1=C[C@H]3CNC[C@@H](C1)O3)c(=O)n2C)c1cccc(C(F)F)c1F]

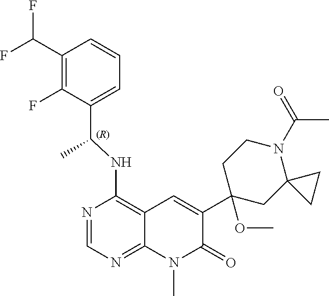

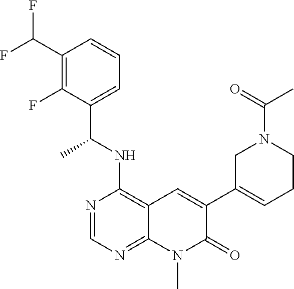 [SMILES: CC(=O)N1CCC=C(c2cc3c(N[C@H](C)c4cccc(C(F)F)c4F)ncnc3n(C)c2=O)C1]

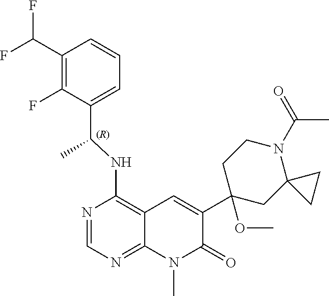

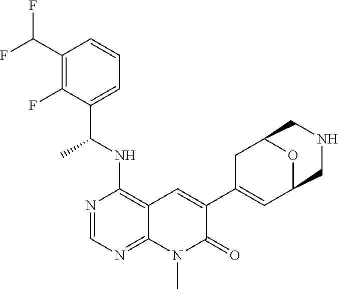 [SMILES: C[C@@H](Nc1ncnc2c1cc(C1=C[C@@H]3CNC[C@H](C1)O3)c(=O)n2C)c1cccc(C(F)F)c1F]

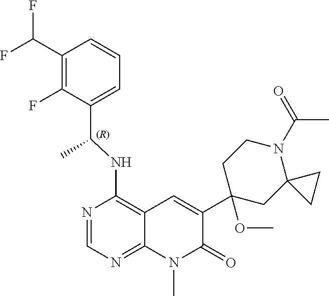

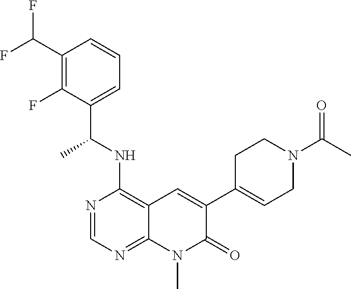 [SMILES: CC(=O)N1CC=C(c2cc3c(N[C@H](C)c4cccc(C(F)F)c4F)ncnc3n(C)c2=O)CC1]

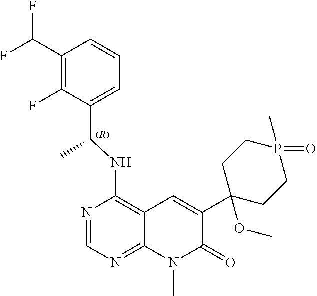

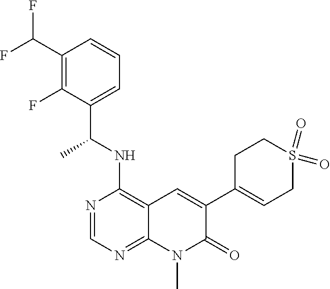 [SMILES: C[C@@H](Nc1ncnc2c1cc(C1=CCS(=O)(=O)CC1)c(=O)n2C)c1cccc(C(F)F)c1F]

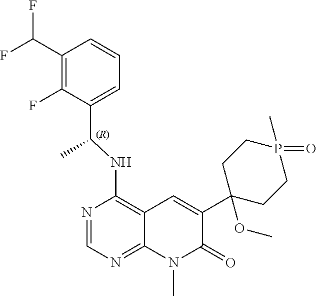

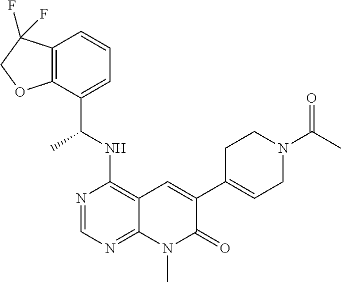 [SMILES: CC(=O)N1CC=C(c2cc3c(N[C@H](C)c4cccc5c4OCC5(F)F)ncnc3n(C)c2=O)CC1]

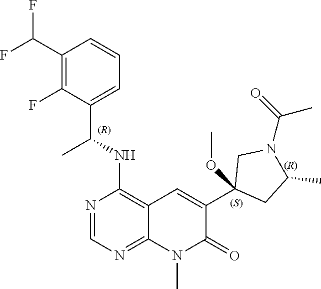

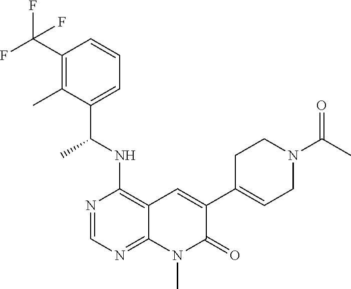 [SMILES: CC(=O)N1CC=C(c2cc3c(N[C@H](C)c4cccc(C(F)(F)F)c4C)ncnc3n(C)c2=O)CC1]

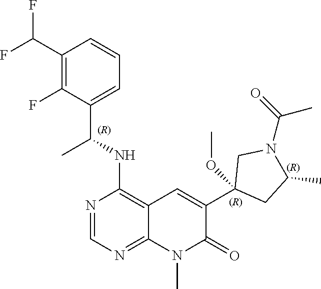

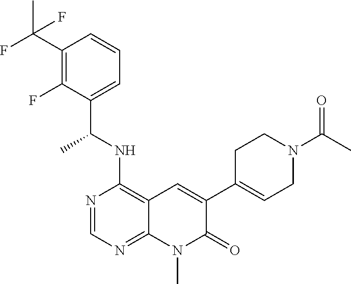 [SMILES: CC(=O)N1CC=C(c2cc3c(N[C@H](C)c4cccc(C(C)(F)F)c4F)ncnc3n(C)c2=O)CC1]

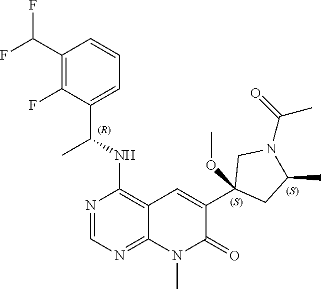

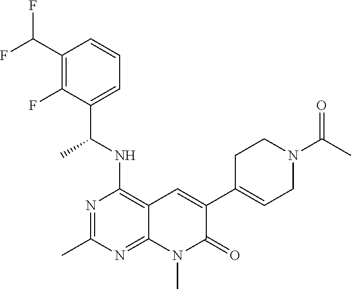 [SMILES: CC(=O)N1CC=C(c2cc3c(N[C@H](C)c4cccc(C(F)F)c4F)nc(C)nc3n(C)c2=O)CC1]

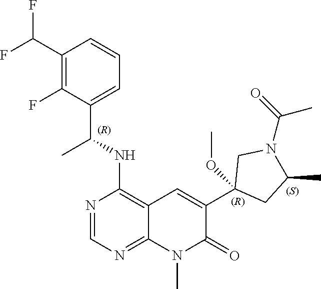

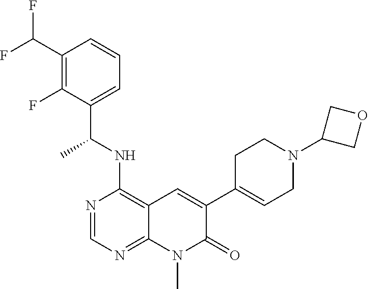 [SMILES: C[C@@H](Nc1ncnc2c1cc(C1=CCN(C3COC3)CC1)c(=O)n2C)c1cccc(C(F)F)c1F]

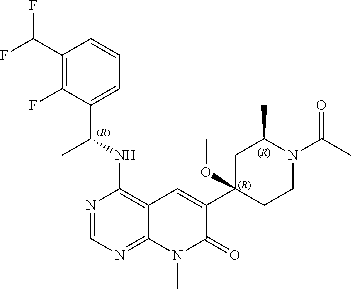

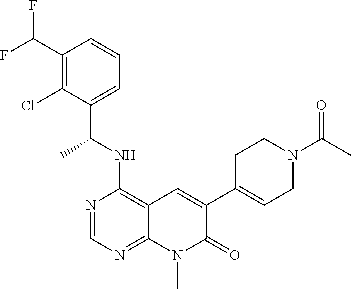 [SMILES: CC(=O)N1CC=C(c2cc3c(N[C@H](C)c4cccc(C(F)F)c4Cl)ncnc3n(C)c2=O)CC1]

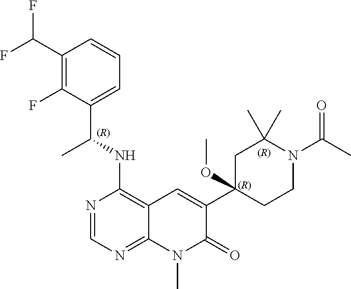

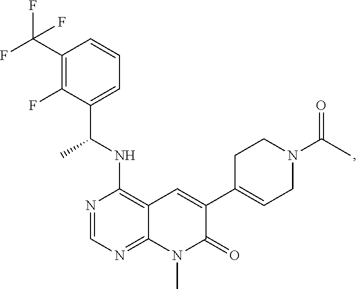 [SMILES: CC(=O)N1CC=C(c2cc3c(N[C@H](C)c4cccc(C(F)(F)F)c4F)ncnc3n(C)c2=O)CC1]

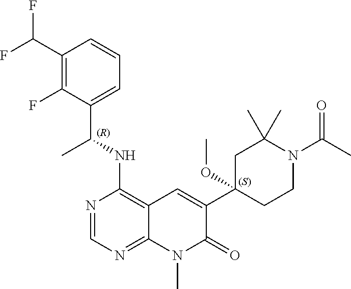

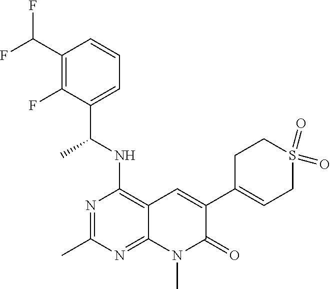 [SMILES: Cc1nc(N[C@H](C)c2cccc(C(F)F)c2F)c2cc(C3=CCS(=O)(=O)CC3)c(=O)n(C)c2n1]

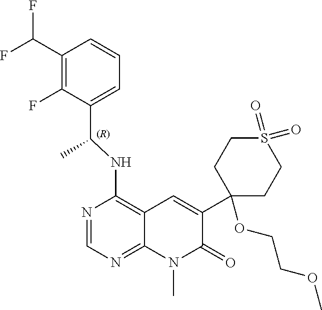

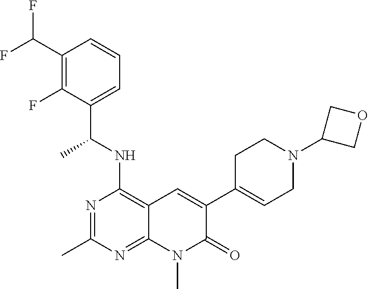 [SMILES: Cc1nc(N[C@H](C)c2cccc(C(F)F)c2F)c2cc(C3=CCN(C4COC4)CC3)c(=O)n(C)c2n1]

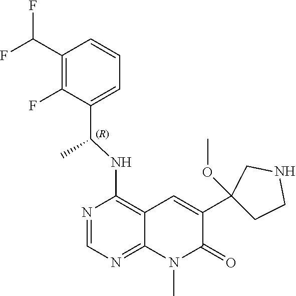

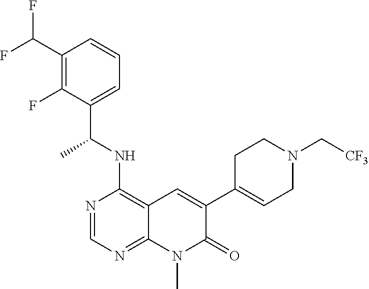 [SMILES: C[C@@H](Nc1ncnc2c1cc(C1=CCN(CC(F)(F)F)CC1)c(=O)n2C)c1cccc(C(F)F)c1F]

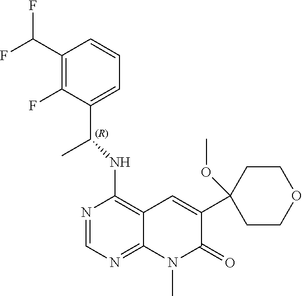

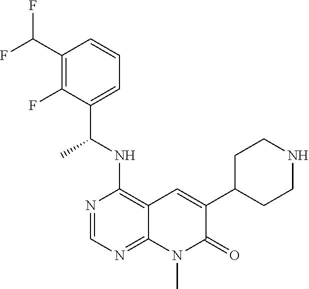 [SMILES: C[C@@H](Nc1ncnc2c1cc(C1CCNCC1)c(=O)n2C)c1cccc(C(F)F)c1F]

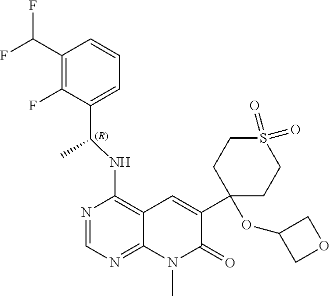

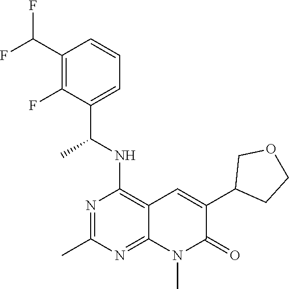 [SMILES: Cc1nc(N[C@H](C)c2cccc(C(F)F)c2F)c2cc(C3CCOC3)c(=O)n(C)c2n1]

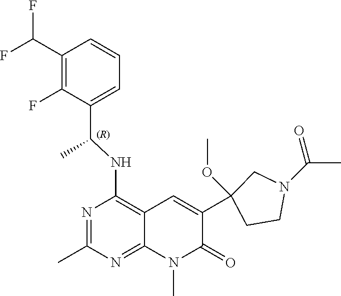

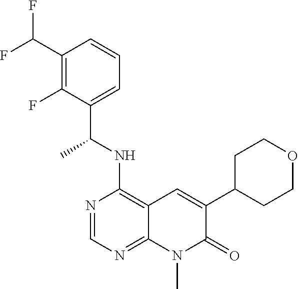 [SMILES: C[C@@H](Nc1ncnc2c1cc(C1CCOCC1)c(=O)n2C)c1cccc(C(F)F)c1F]

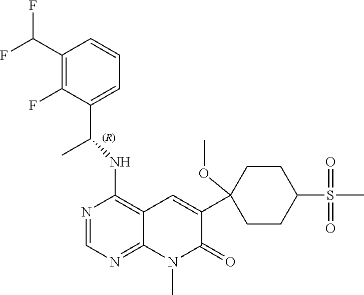

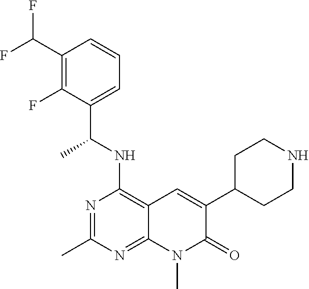 [SMILES: Cc1nc(N[C@H](C)c2cccc(C(F)F)c2F)c2cc(C3CCNCC3)c(=O)n(C)c2n1]

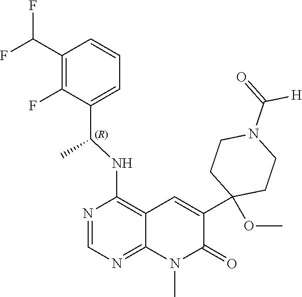

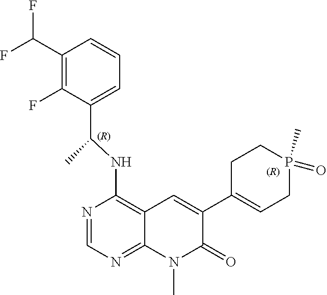

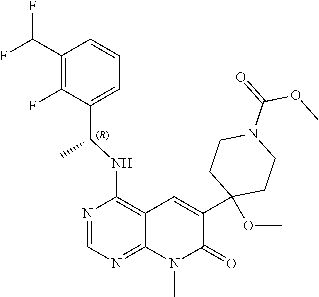

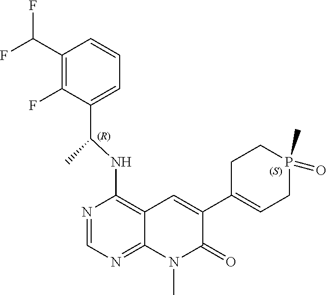

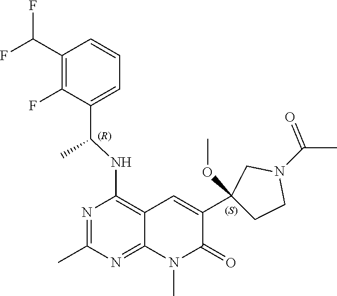

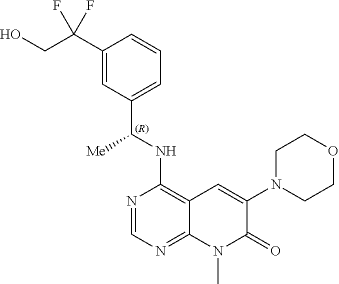

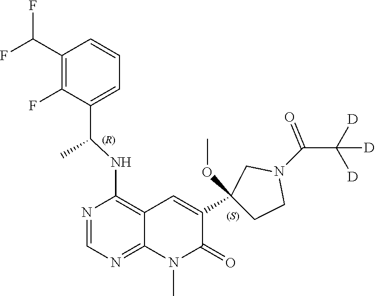

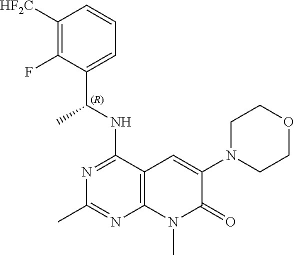

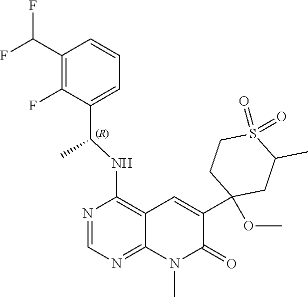

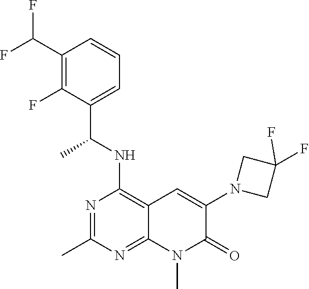 [SMILES: Cc1nc(N[C@H](C)c2cccc(C(F)F)c2F)c2cc(N3CC(F)(F)C3)c(=O)n(C)c2n1]

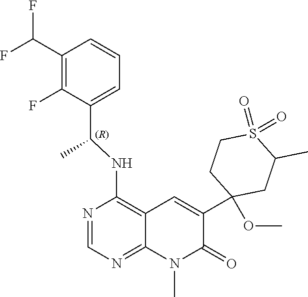

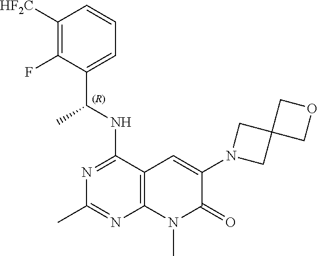

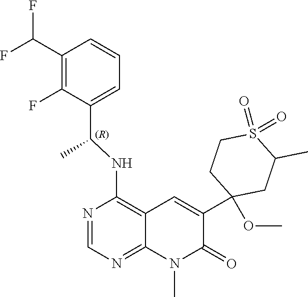

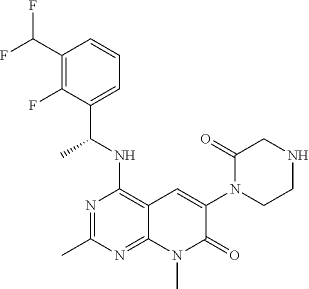 [SMILES: Cc1nc(N[C@H](C)c2cccc(C(F)F)c2F)c2cc(N3CCNCC3=O)c(=O)n(C)c2n1]

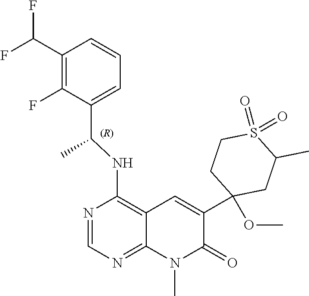

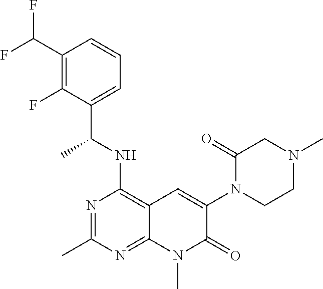 [SMILES: Cc1nc(N[C@H](C)c2cccc(C(F)F)c2F)c2cc(N3CCN(C)CC3=O)c(=O)n(C)c2n1]

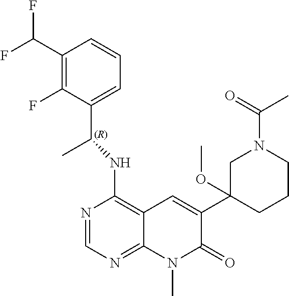

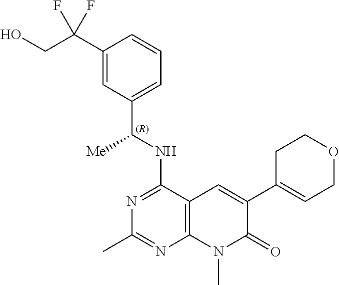

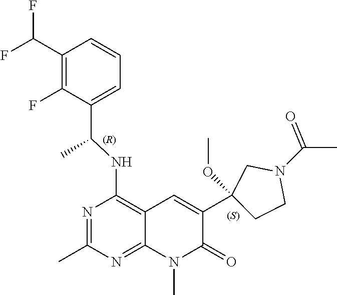

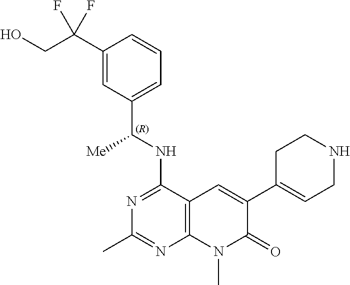

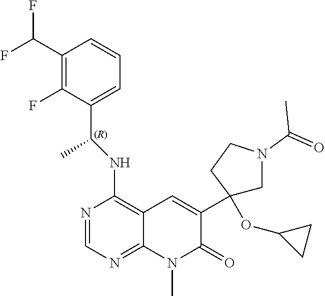

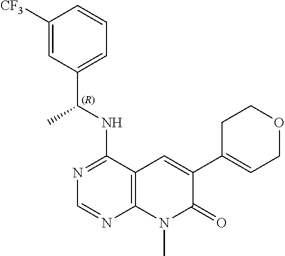

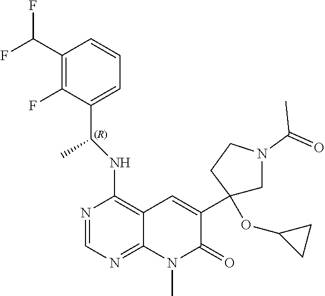

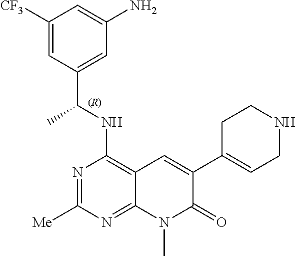

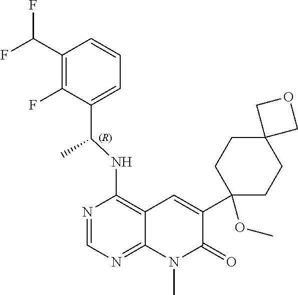

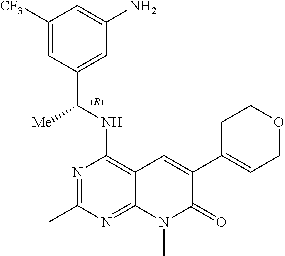

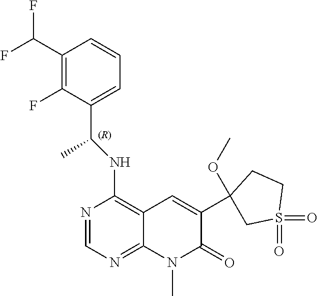

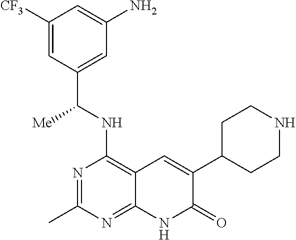 [SMILES: Cc1nc(N[C@H](C)c2cc(N)cc(C(F)(F)F)c2)c2cc(C3CCNCC3)c(=O)[nH]c2n1]

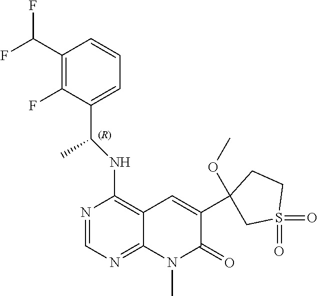

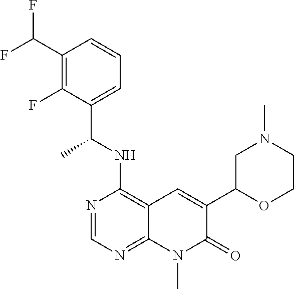 [SMILES: C[C@@H](Nc1ncnc2c1cc(C1CN(C)CCO1)c(=O)n2C)c1cccc(C(F)F)c1F]

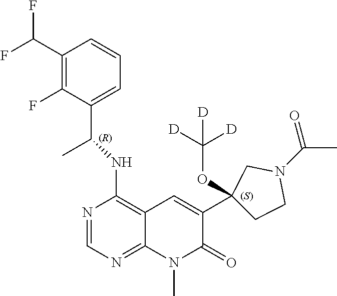

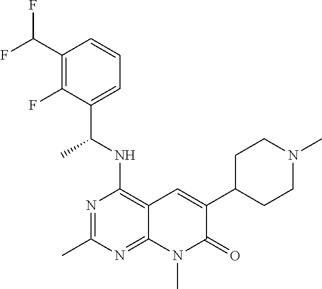 [SMILES: Cc1nc(N[C@H](C)c2cccc(C(F)F)c2F)c2cc(C3CCN(C)CC3)c(=O)n(C)c2n1]

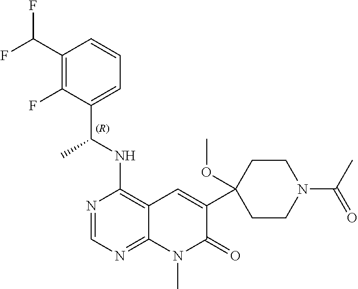

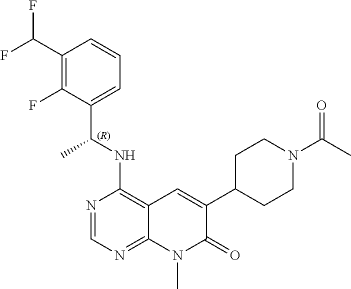

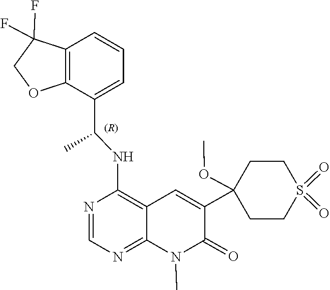

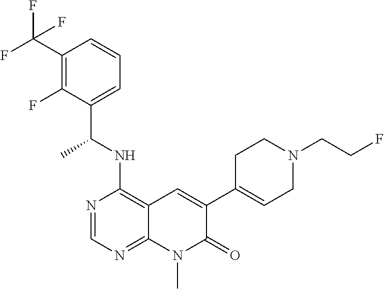 [SMILES: C[C@@H](Nc1ncnc2c1cc(C1=CCN(CCF)CC1)c(=O)n2C)c1cccc(C(F)(F)F)c1F]

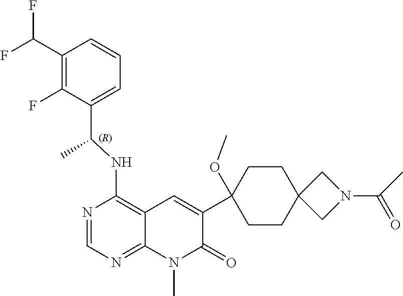

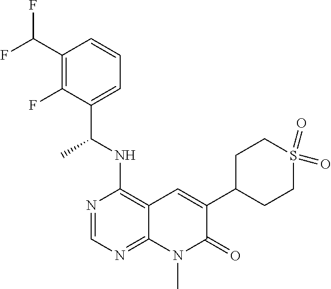 [SMILES: C[C@@H](Nc1ncnc2c1cc(C1CCS(=O)(=O)CC1)c(=O)n2C)c1cccc(C(F)F)c1F]

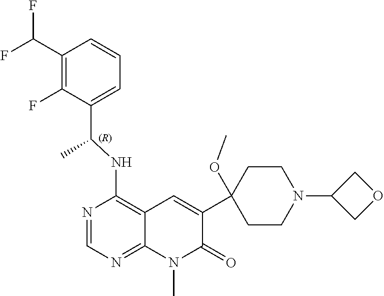

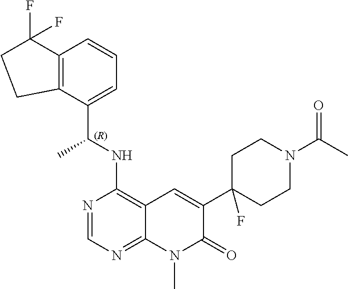

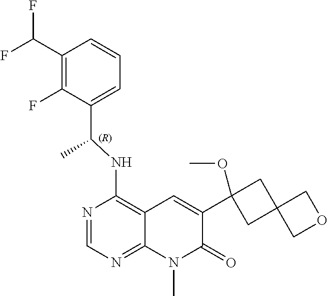

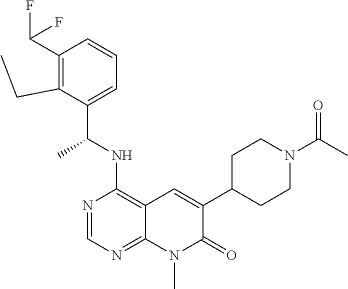 [SMILES: CCc1c(C(F)F)cccc1[C@@H](C)Nc1ncnc2c1cc(C1CCN(C(C)=O)CC1)c(=O)n2C]

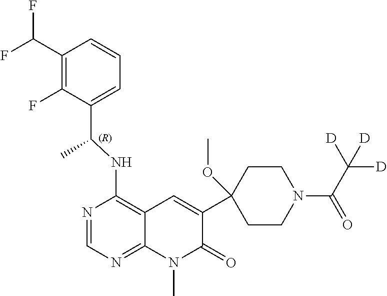

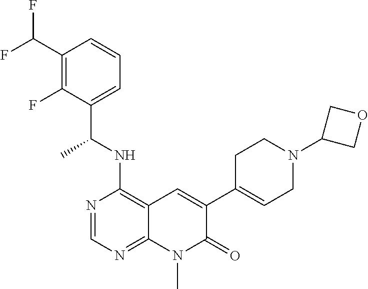 [SMILES: C[C@@H](Nc1ncnc2c1cc(C1=CCN(C3COC3)CC1)c(=O)n2C)c1cccc(C(F)F)c1F]

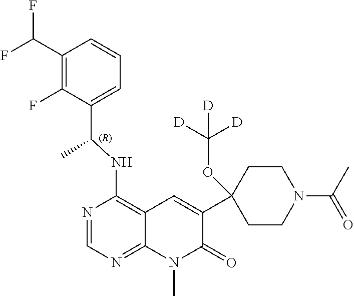

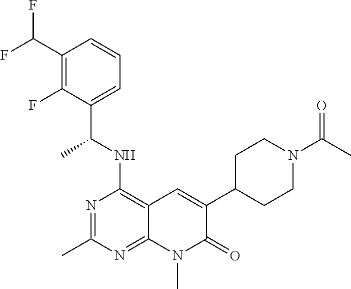 [SMILES: CC(=O)N1CCC(c2cc3c(N[C@H](C)c4cccc(C(F)F)c4F)nc(C)nc3n(C)c2=O)CC1]

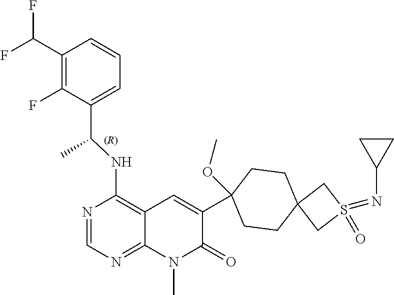

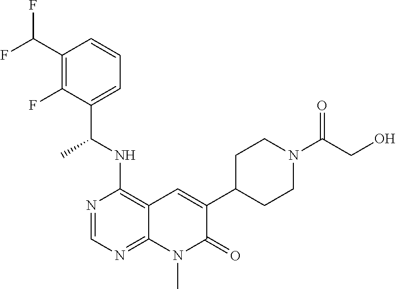 [SMILES: C[C@@H](Nc1ncnc2c1cc(C1CCN(C(=O)CO)CC1)c(=O)n2C)c1cccc(C(F)F)c1F]

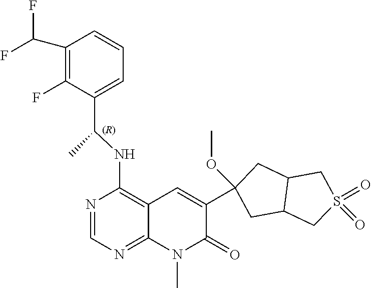

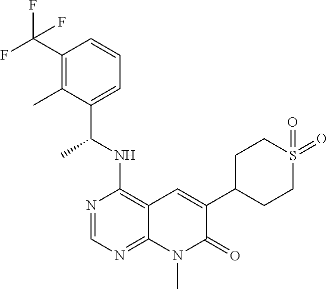 [SMILES: Cc1c([C@@H](C)Nc2ncnc3c2cc(C2CCS(=O)(=O)CC2)c(=O)n3C)cccc1C(F)(F)F]

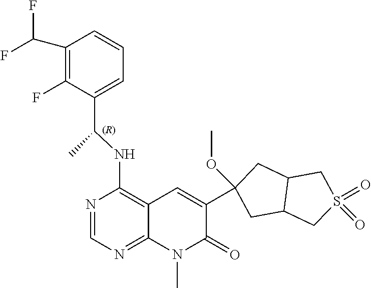

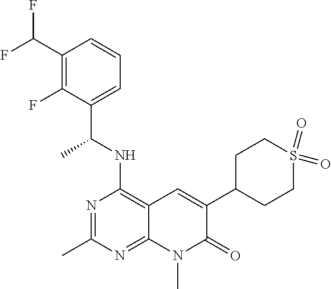 [SMILES: Cc1nc(N[C@H](C)c2cccc(C(F)F)c2F)c2cc(C3CCS(=O)(=O)CC3)c(=O)n(C)c2n1]

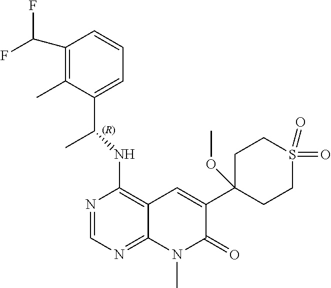

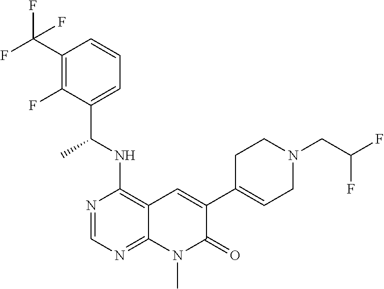 [SMILES: C[C@@H](Nc1ncnc2c1cc(C1=CCN(CC(F)F)CC1)c(=O)n2C)c1cccc(C(F)(F)F)c1F]

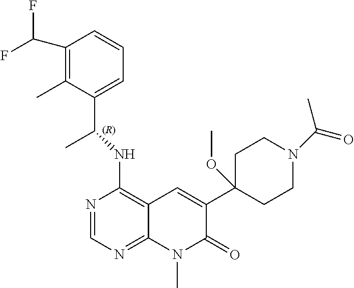

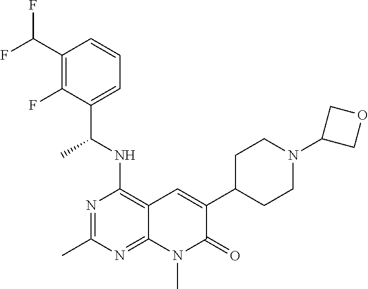 [SMILES: Cc1nc(N[C@H](C)c2cccc(C(F)F)c2F)c2cc(C3CCN(C4COC4)CC3)c(=O)n(C)c2n1]

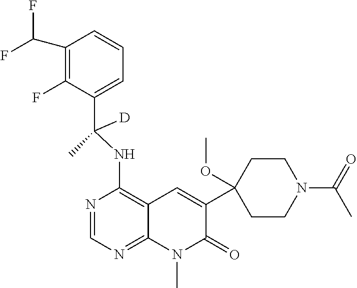 [SMILES: [2H][C@@](C)(Nc1ncnc2c1cc(C1(OC)CCN(C(C)=O)CC1)c(=O)n2C)c1cccc(C(F)F)c1F]

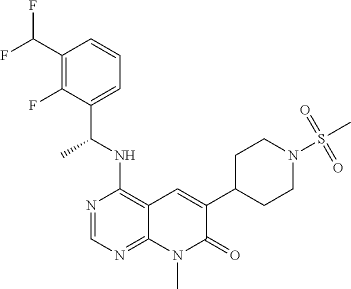 [SMILES: C[C@@H](Nc1ncnc2c1cc(C1CCN(S(C)(=O)=O)CC1)c(=O)n2C)c1cccc(C(F)F)c1F]

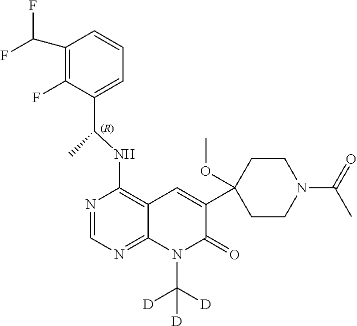

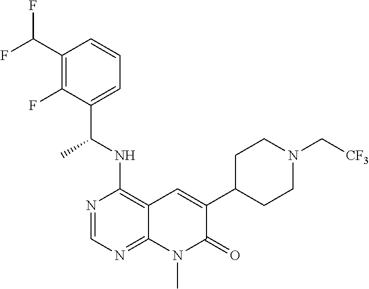 [SMILES: C[C@@H](Nc1ncnc2c1cc(C1CCN(CC(F)(F)F)CC1)c(=O)n2C)c1cccc(C(F)F)c1F]

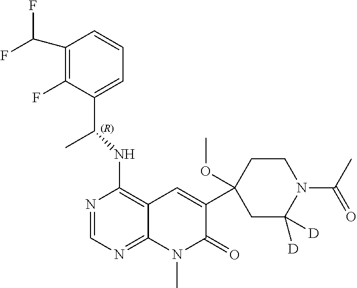

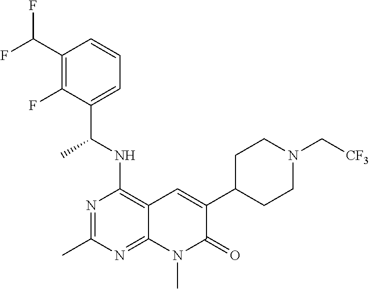 [SMILES: Cc1nc(N[C@H](C)c2cccc(C(F)F)c2F)c2cc(C3CCN(CC(F)(F)F)CC3)c(=O)n(C)c2n1]

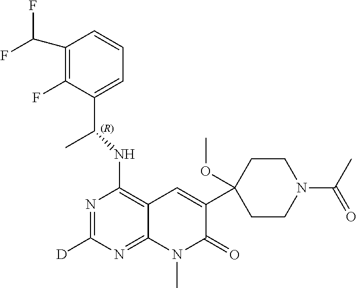

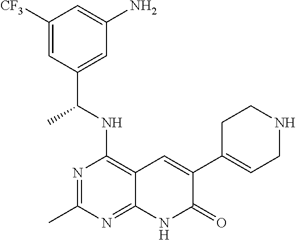 [SMILES: Cc1nc(N[C@H](C)c2cc(N)cc(C(F)(F)F)c2)c2cc(C3=CCNCC3)c(=O)[nH]c2n1]

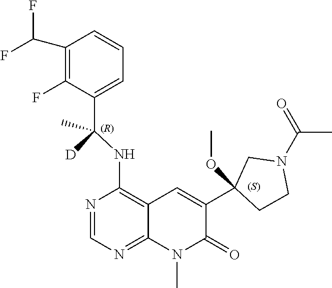

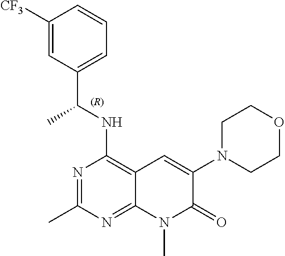

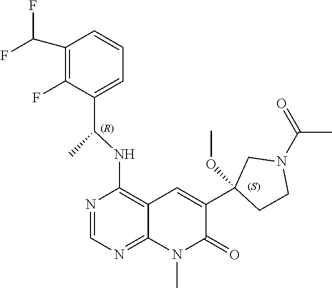

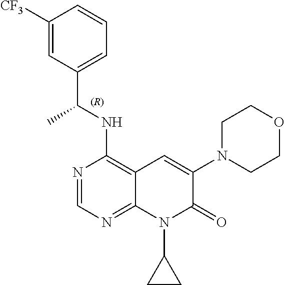

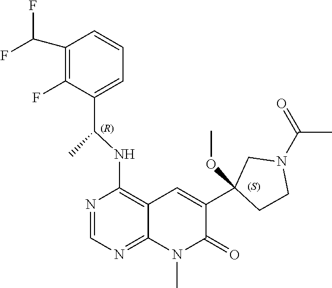

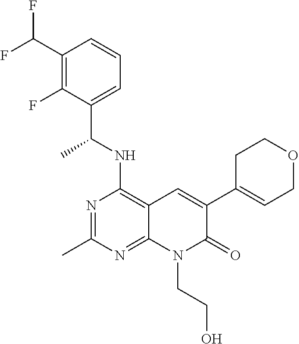 [SMILES: Cc1nc(N[C@H](C)c2cccc(C(F)F)c2F)c2cc(C3=CCOCC3)c(=O)n(CCO)c2n1]

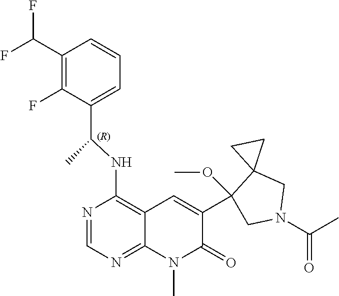

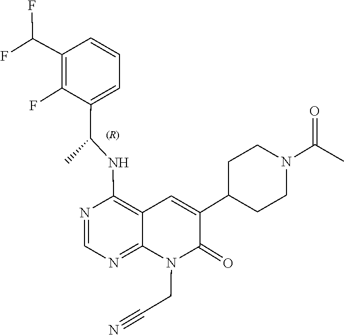

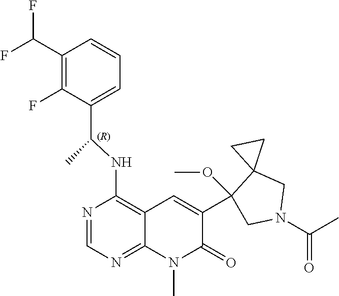

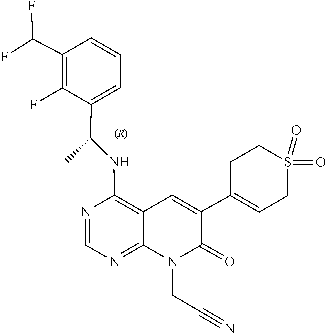

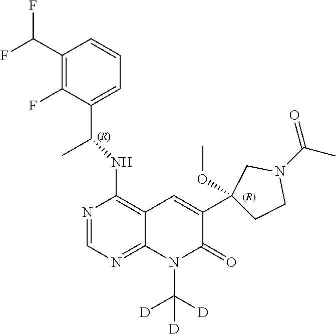

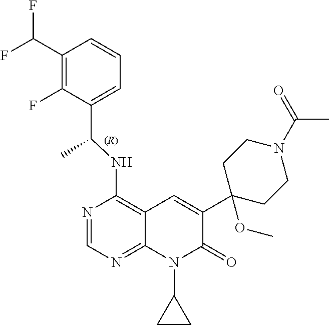

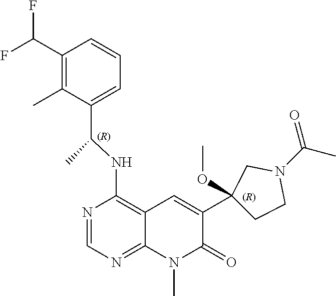

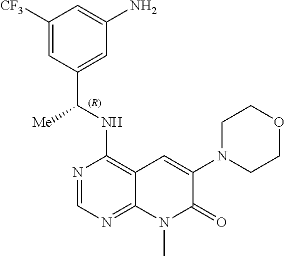

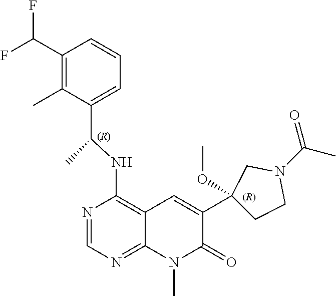

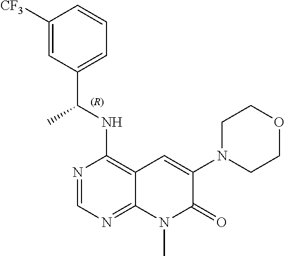

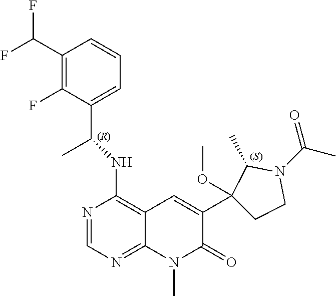

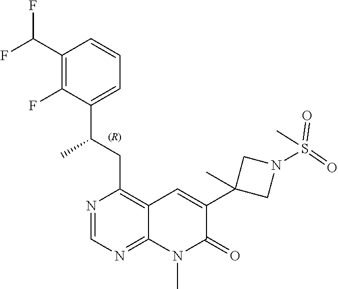

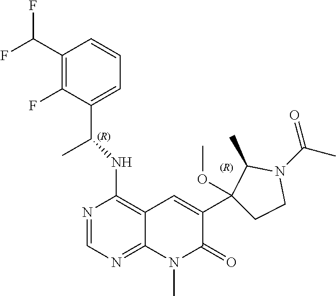

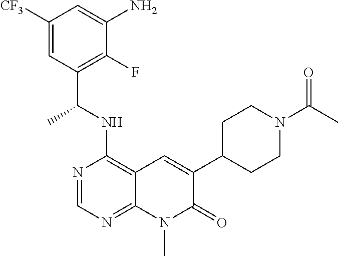 [SMILES: CC(=O)N1CCC(c2cc3c(N[C@H](C)c4cc(C(F)(F)F)cc(N)c4F)ncnc3n(C)c2=O)CC1]

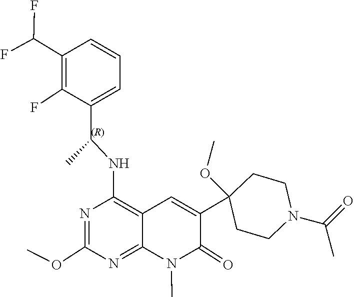

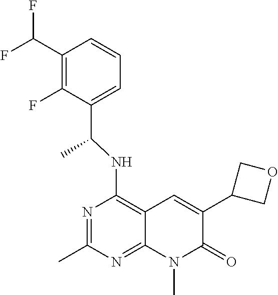 [SMILES: Cc1nc(N[C@H](C)c2cccc(C(F)F)c2F)c2cc(C3COC3)c(=O)n(C)c2n1]

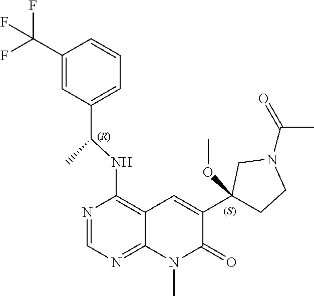

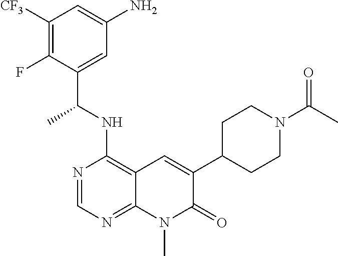 [SMILES: CC(=O)N1CCC(c2cc3c(N[C@H](C)c4cc(N)cc(C(F)(F)F)c4F)ncnc3n(C)c2=O)CC1]

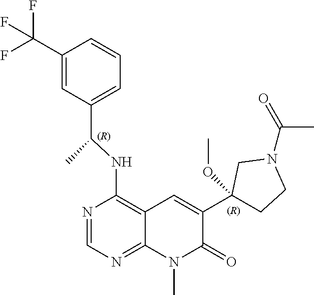

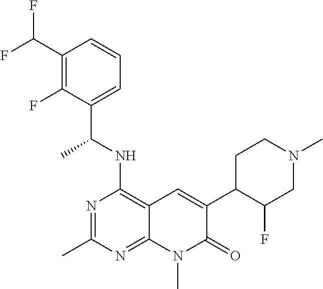 [SMILES: Cc1nc(N[C@H](C)c2cccc(C(F)F)c2F)c2cc(C3CCN(C)CC3F)c(=O)n(C)c2n1]

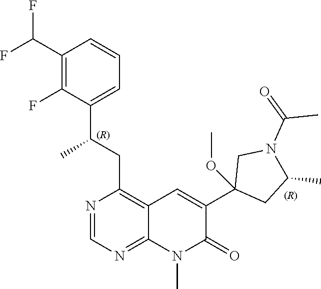

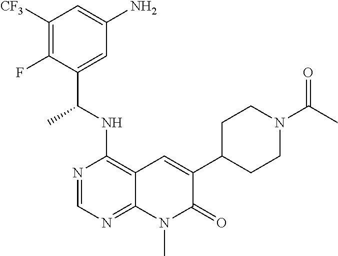 [SMILES: CC(=O)N1CCC(c2cc3c(N[C@H](C)c4cc(N)cc(C(F)(F)F)c4F)ncnc3n(C)c2=O)CC1]

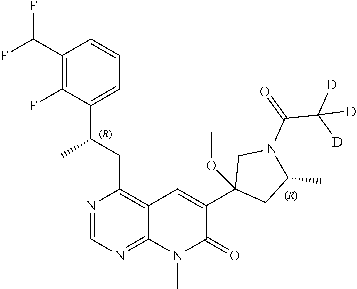

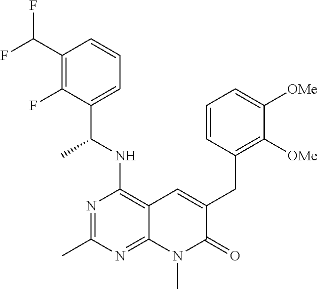 [SMILES: COc1cccc(Cc2cc3c(N[C@H](C)c4cccc(C(F)F)c4F)nc(C)nc3n(C)c2=O)c1OC]

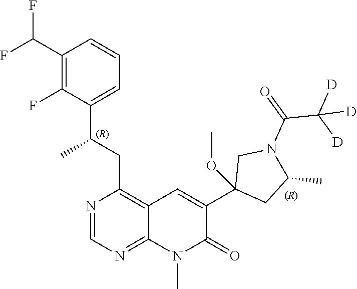

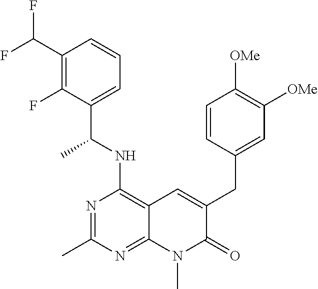 [SMILES: COc1ccc(Cc2cc3c(N[C@H](C)c4cccc(C(F)F)c4F)nc(C)nc3n(C)c2=O)cc1OC]

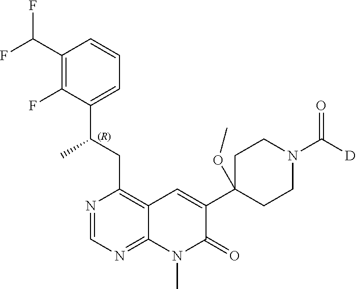

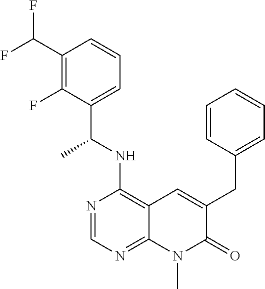 [SMILES: C[C@@H](Nc1ncnc2c1cc(Cc1ccccc1)c(=O)n2C)c1cccc(C(F)F)c1F]

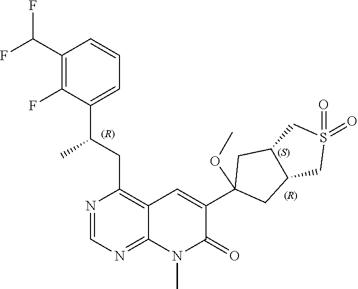

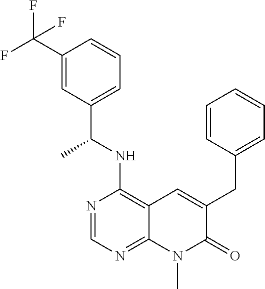 [SMILES: C[C@@H](Nc1ncnc2c1cc(Cc1ccccc1)c(=O)n2C)c1cccc(C(F)(F)F)c1]

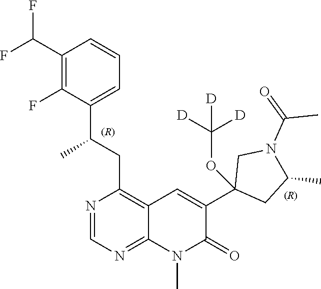

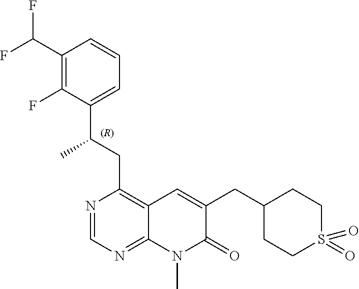

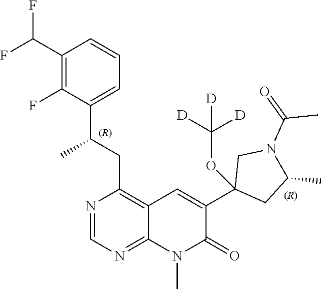

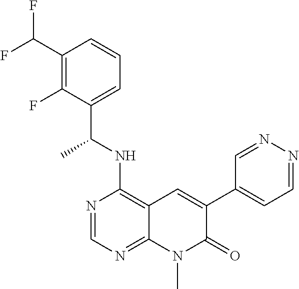 [SMILES: C[C@@H](Nc1ncnc2c1cc(-c1ccnnc1)c(=O)n2C)c1cccc(C(F)F)c1F]

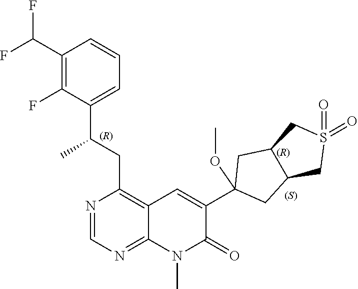

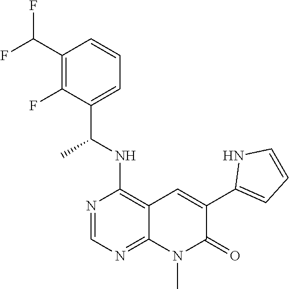 [SMILES: C[C@@H](Nc1ncnc2c1cc(-c1ccc[nH]1)c(=O)n2C)c1cccc(C(F)F)c1F]

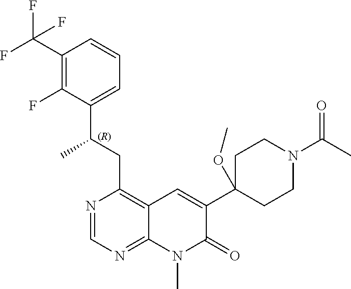

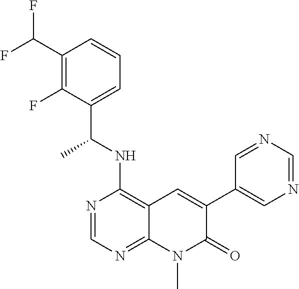 [SMILES: C[C@@H](Nc1ncnc2c1cc(-c1cncnc1)c(=O)n2C)c1cccc(C(F)F)c1F]

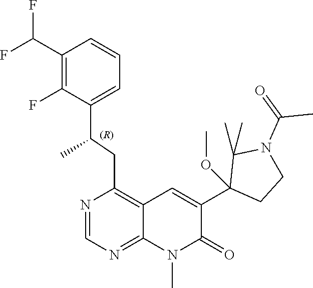

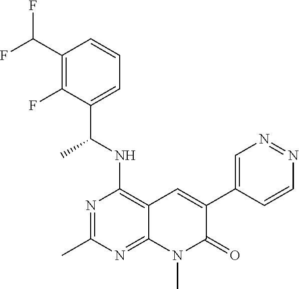 [SMILES: Cc1nc(N[C@H](C)c2cccc(C(F)F)c2F)c2cc(-c3ccnnc3)c(=O)n(C)c2n1]

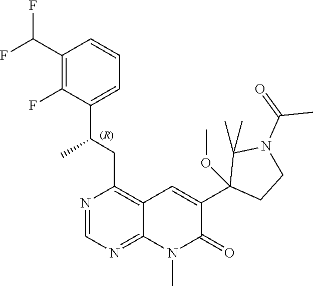

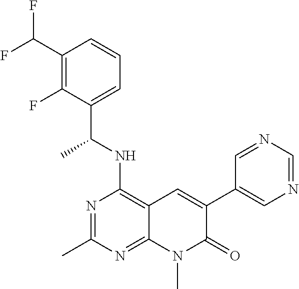 [SMILES: Cc1nc(N[C@H](C)c2cccc(C(F)F)c2F)c2cc(-c3cncnc3)c(=O)n(C)c2n1]

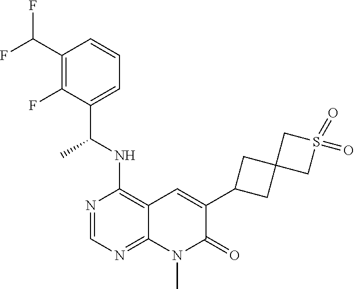 [SMILES: C[C@@H](Nc1ncnc2c1cc(C1CC3(C1)CS(=O)(=O)C3)c(=O)n2C)c1cccc(C(F)F)c1F]

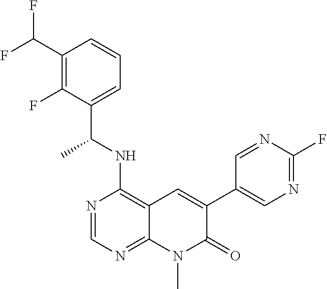 [SMILES: C[C@@H](Nc1ncnc2c1cc(-c1cnc(F)nc1)c(=O)n2C)c1cccc(C(F)F)c1F]

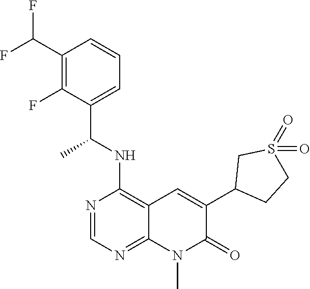 [SMILES: C[C@@H](Nc1ncnc2c1cc(C1CCS(=O)(=O)C1)c(=O)n2C)c1cccc(C(F)F)c1F]

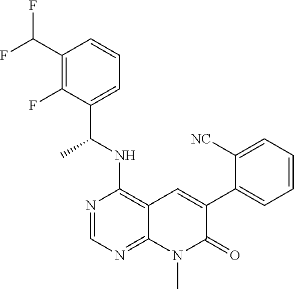 [SMILES: C[C@@H](Nc1ncnc2c1cc(-c1ccccc1C#N)c(=O)n2C)c1cccc(C(F)F)c1F]

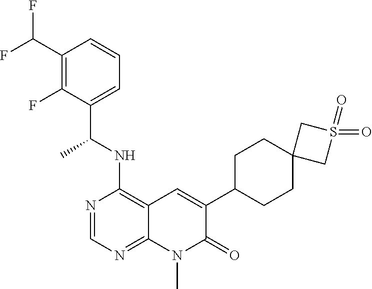 [SMILES: C[C@@H](Nc1ncnc2c1cc(C1CCC3(CC1)CS(=O)(=O)C3)c(=O)n2C)c1cccc(C(F)F)c1F]

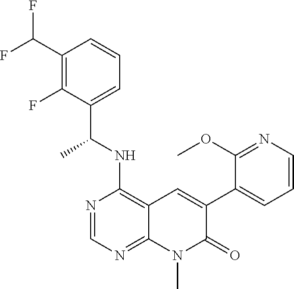 [SMILES: COc1ncccc1-c1cc2c(N[C@H](C)c3cccc(C(F)F)c3F)ncnc2n(C)c1=O]

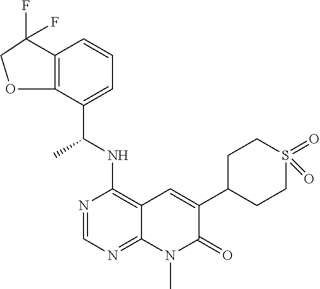 [SMILES: C[C@@H](Nc1ncnc2c1cc(C1CCS(=O)(=O)CC1)c(=O)n2C)c1cccc2c1OCC2(F)F]

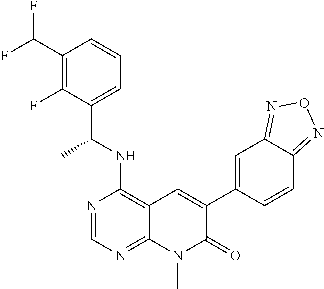 [SMILES: C[C@@H](Nc1ncnc2c1cc(-c1ccc3nonc3c1)c(=O)n2C)c1cccc(C(F)F)c1F]

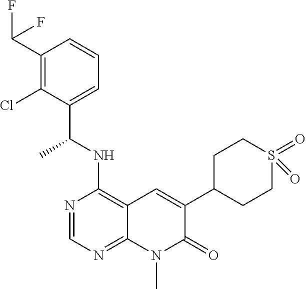 [SMILES: C[C@@H](Nc1ncnc2c1cc(C1CCS(=O)(=O)CC1)c(=O)n2C)c1cccc(C(F)F)c1Cl]

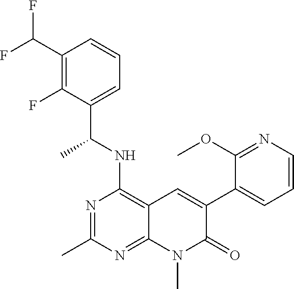 [SMILES: COc1ncccc1-c1cc2c(N[C@H](C)c3cccc(C(F)F)c3F)nc(C)nc2n(C)c1=O]

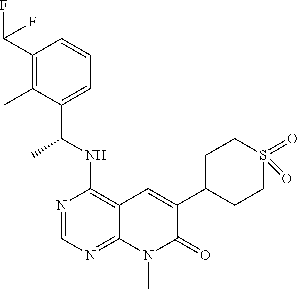 [SMILES: Cc1c(C(F)F)cccc1[C@@H](C)Nc1ncnc2c1cc(C1CCS(=O)(=O)CC1)c(=O)n2C]

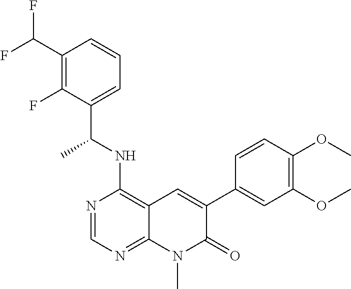 [SMILES: COc1ccc(-c2cc3c(N[C@H](C)c4cccc(C(F)F)c4F)ncnc3n(C)c2=O)cc1OC]

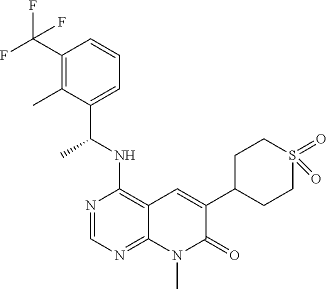 [SMILES: Cc1c([C@@H](C)Nc2ncnc3c2cc(C2CCS(=O)(=O)CC2)c(=O)n3C)cccc1C(F)(F)F]

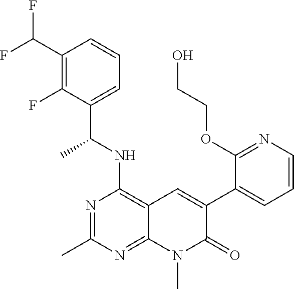 [SMILES: Cc1nc(N[C@H](C)c2cccc(C(F)F)c2F)c2cc(-c3cccnc3OCCO)c(=O)n(C)c2n1]

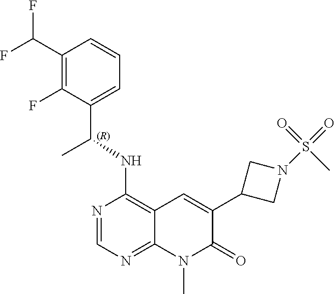

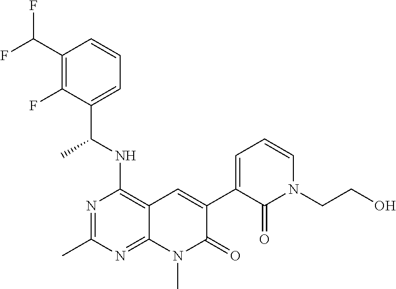 [SMILES: Cc1nc(N[C@H](C)c2cccc(C(F)F)c2F)c2cc(-c3cccn(CCO)c3=O)c(=O)n(C)c2n1]

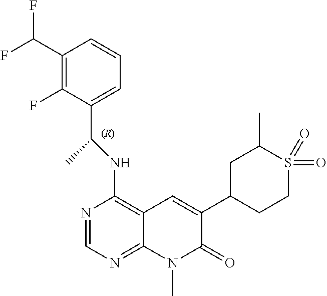

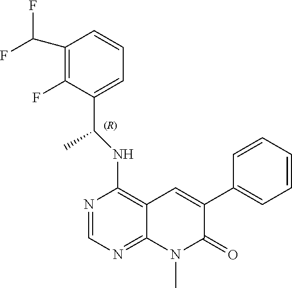

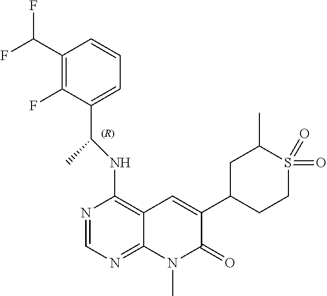

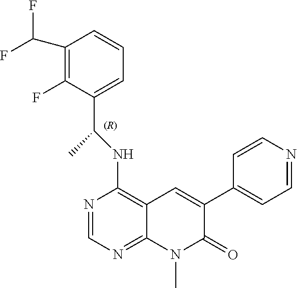

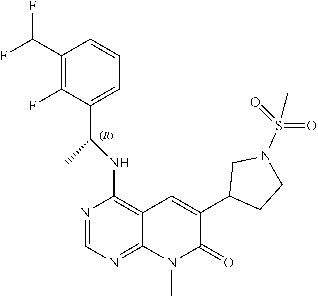

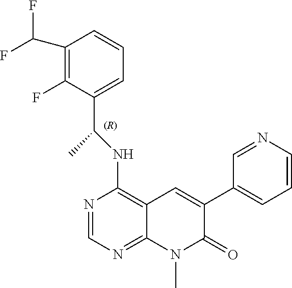

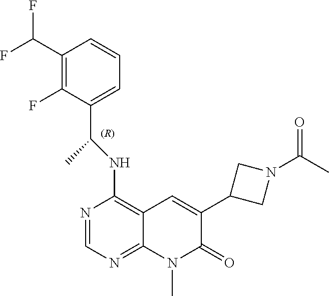

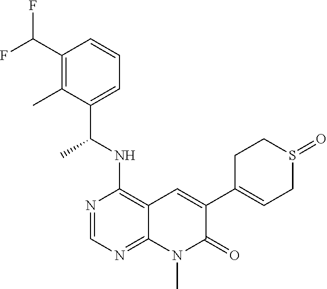 [SMILES: Cc1c(C(F)F)cccc1[C@@H](C)Nc1ncnc2c1cc(C1=CCS(=O)CC1)c(=O)n2C]

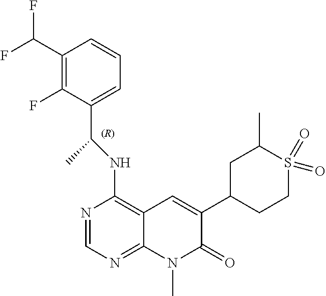

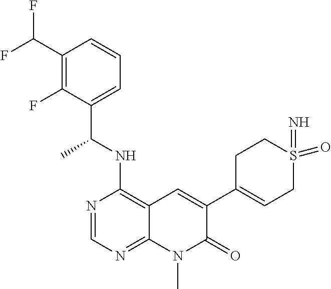 [SMILES: C[C@@H](Nc1ncnc2c1cc(C1=CCS(=N)(=O)CC1)c(=O)n2C)c1cccc(C(F)F)c1F]

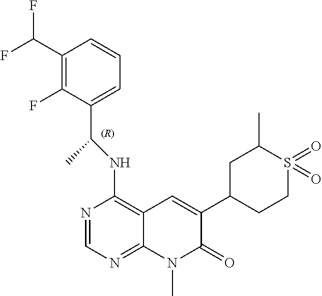

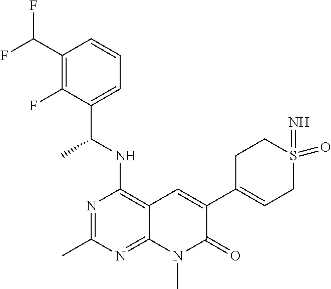 [SMILES: Cc1nc(N[C@H](C)c2cccc(C(F)F)c2F)c2cc(C3=CCS(=N)(=O)CC3)c(=O)n(C)c2n1]

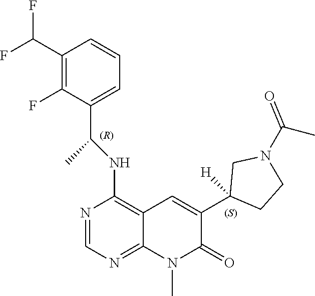

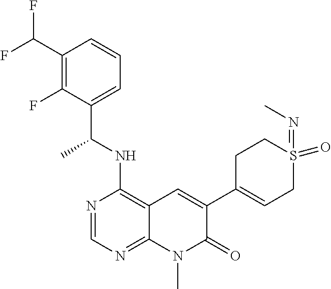 [SMILES: CN=S1(=O)CC=C(c2cc3c(N[C@H](C)c4cccc(C(F)F)c4F)ncnc3n(C)c2=O)CC1]

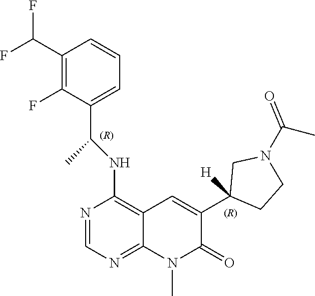

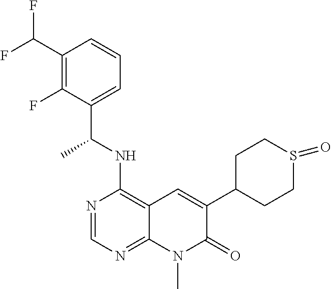 [SMILES: C[C@@H](Nc1ncnc2c1cc(C1CCS(=O)CC1)c(=O)n2C)c1cccc(C(F)F)c1F]

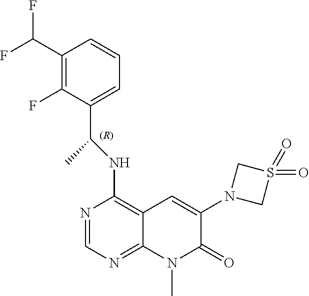

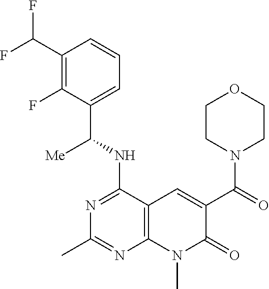 [SMILES: Cc1nc(N[C@H](C)c2cccc(C(F)F)c2F)c2cc(C(=O)N3CCOCC3)c(=O)n(C)c2n1]

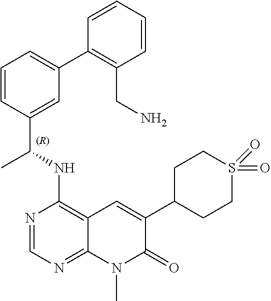

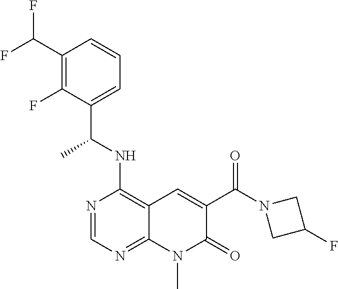 [SMILES: C[C@@H](Nc1ncnc2c1cc(C(=O)N1CC(F)C1)c(=O)n2C)c1cccc(C(F)F)c1F]

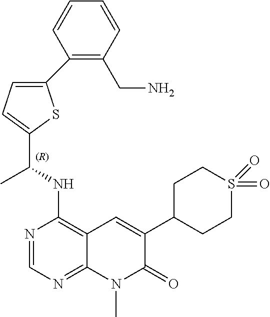

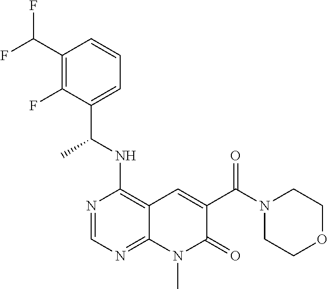 [SMILES: C[C@@H](Nc1ncnc2c1cc(C(=O)N1CCOCC1)c(=O)n2C)c1cccc(C(F)F)c1F]

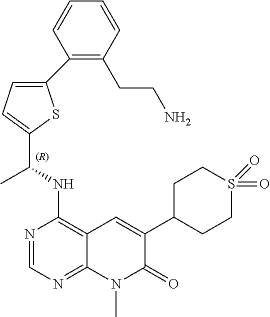

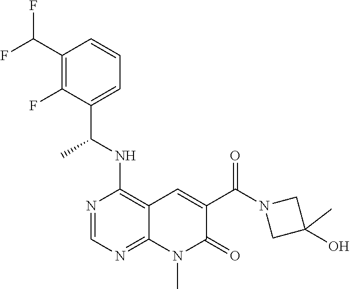 [SMILES: C[C@@H](Nc1ncnc2c1cc(C(=O)N1CC(C)(O)C1)c(=O)n2C)c1cccc(C(F)F)c1F]

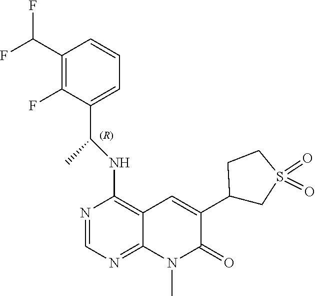

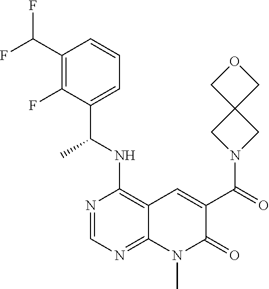 [SMILES: C[C@@H](Nc1ncnc2c1cc(C(=O)N1CC3(COC3)C1)c(=O)n2C)c1cccc(C(F)F)c1F]

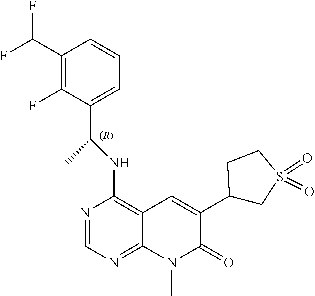

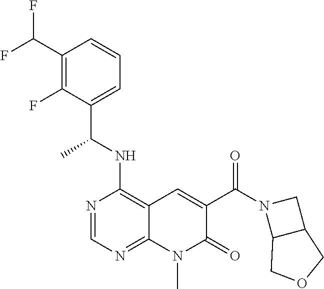 [SMILES: C[C@@H](Nc1ncnc2c1cc(C(=O)N1CC3COCC31)c(=O)n2C)c1cccc(C(F)F)c1F]

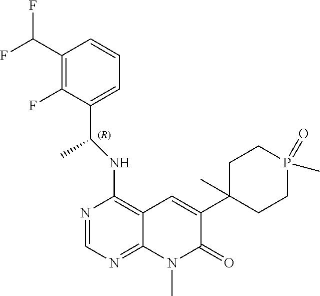

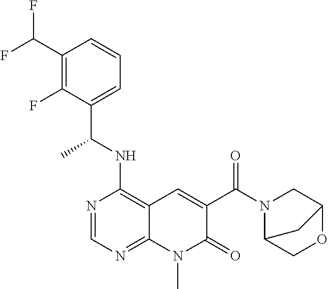 [SMILES: C[C@@H](Nc1ncnc2c1cc(C(=O)N1CC3CC1CO3)c(=O)n2C)c1cccc(C(F)F)c1F]

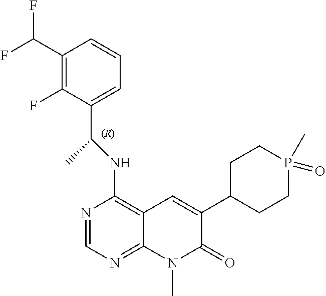

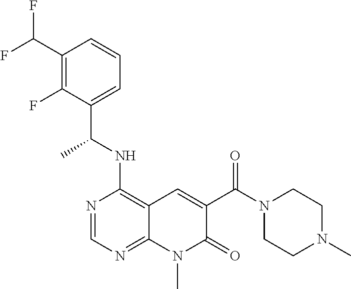 [SMILES: C[C@@H](Nc1ncnc2c1cc(C(=O)N1CCN(C)CC1)c(=O)n2C)c1cccc(C(F)F)c1F]

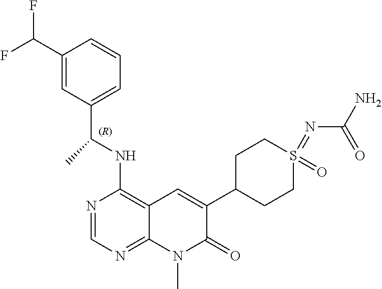

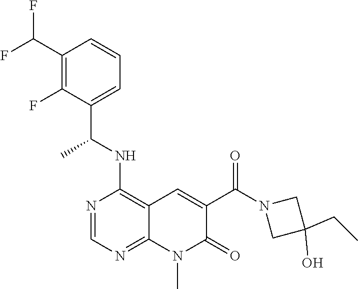 [SMILES: CCC1(O)CN(C(=O)c2cc3c(N[C@H](C)c4cccc(C(F)F)c4F)ncnc3n(C)c2=O)C1]

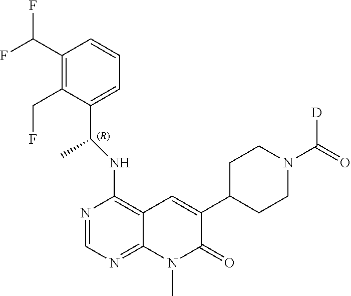

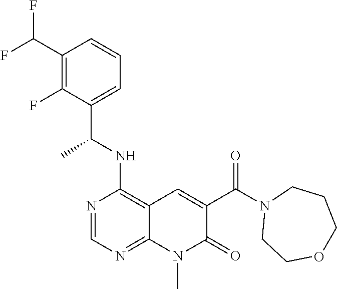 [SMILES: C[C@@H](Nc1ncnc2c1cc(C(=O)N1CCCOCC1)c(=O)n2C)c1cccc(C(F)F)c1F]

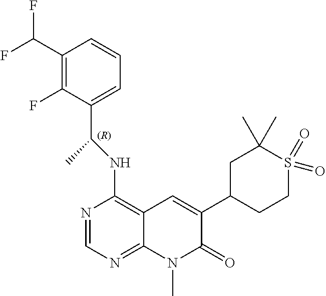

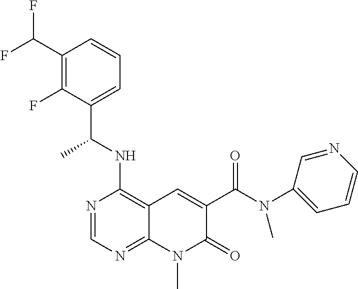 [SMILES: C[C@@H](Nc1ncnc2c1cc(C(=O)N(C)c1cccnc1)c(=O)n2C)c1cccc(C(F)F)c1F]

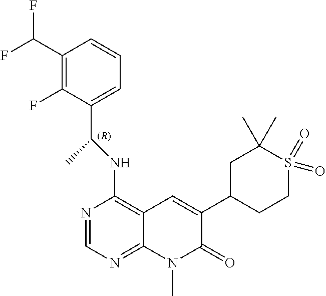

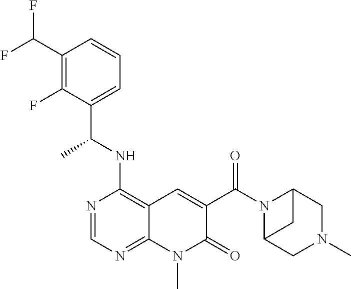 [SMILES: C[C@@H](Nc1ncnc2c1cc(C(=O)N1C3CC1CN(C)C3)c(=O)n2C)c1cccc(C(F)F)c1F]

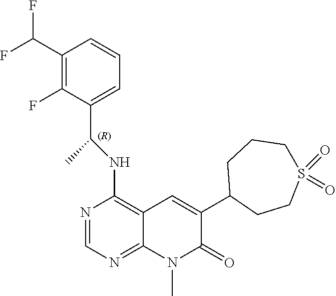

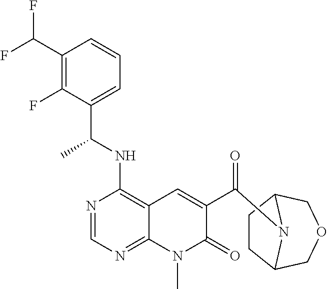 [SMILES: C[C@@H](Nc1ncnc2c1cc(C(=O)N1C3CCC1COC3)c(=O)n2C)c1cccc(C(F)F)c1F]

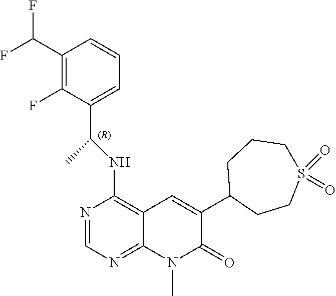

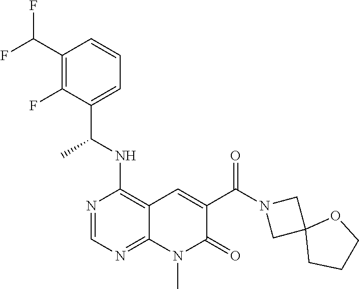 [SMILES: C[C@@H](Nc1ncnc2c1cc(C(=O)N1CC3(CCCO3)C1)c(=O)n2C)c1cccc(C(F)F)c1F]

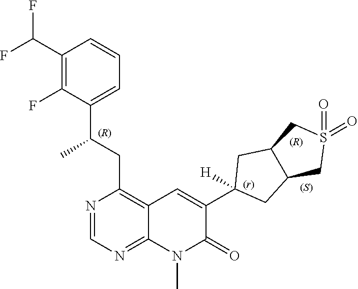

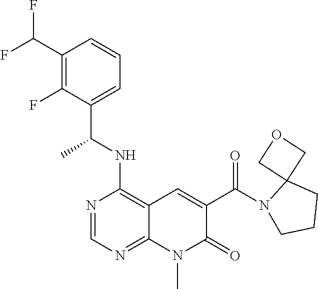 [SMILES: C[C@@H](Nc1ncnc2c1cc(C(=O)N1CCCC13COC3)c(=O)n2C)c1cccc(C(F)F)c1F]

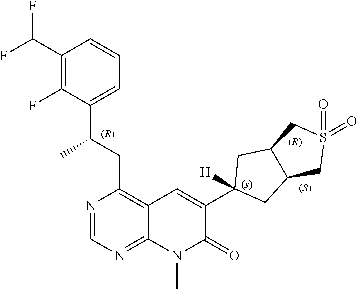

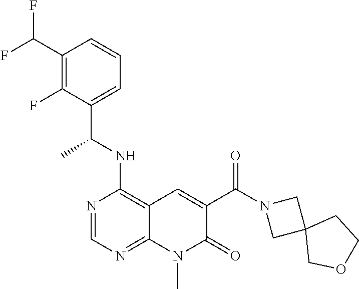 [SMILES: C[C@@H](Nc1ncnc2c1cc(C(=O)N1CC3(CCOC3)C1)c(=O)n2C)c1cccc(C(F)F)c1F]

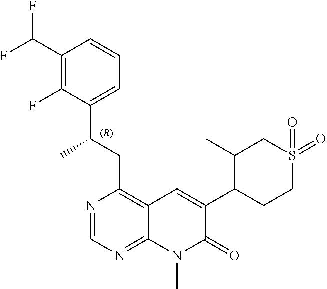

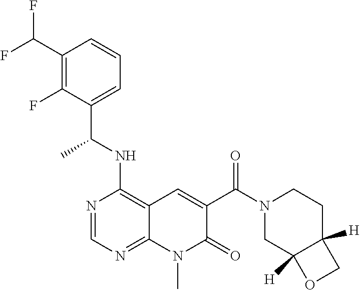 [SMILES: C[C@@H](Nc1ncnc2c1cc(C(=O)N1CC[C@@H]3CO[C@@H]3C1)c(=O)n2C)c1cccc(C(F)F)c1F]

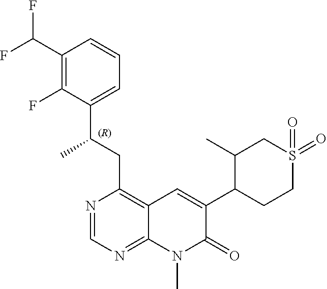

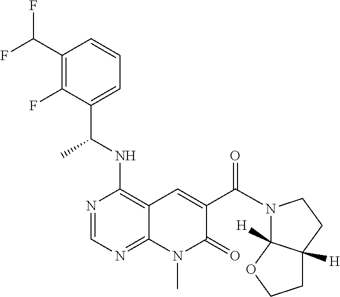 [SMILES: C[C@@H](Nc1ncnc2c1cc(C(=O)N1CC[C@@H]3CCO[C@@H]31)c(=O)n2C)c1cccc(C(F)F)c1F]

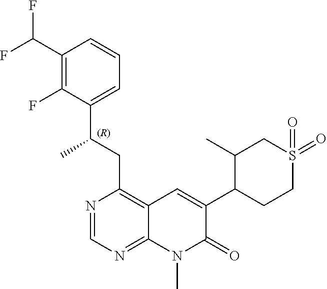

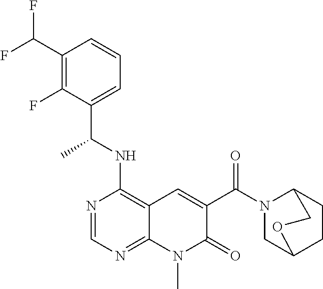 [SMILES: C[C@@H](Nc1ncnc2c1cc(C(=O)N1CC3CCC1CO3)c(=O)n2C)c1cccc(C(F)F)c1F]

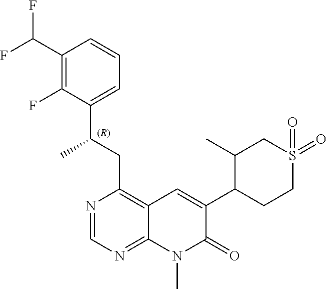

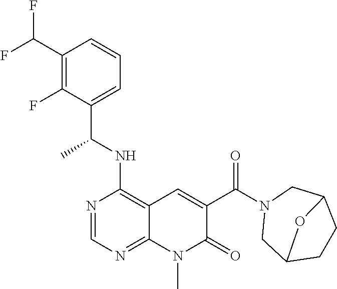 [SMILES: C[C@@H](Nc1ncnc2c1cc(C(=O)N1CC3CCC(C1)O3)c(=O)n2C)c1cccc(C(F)F)c1F]

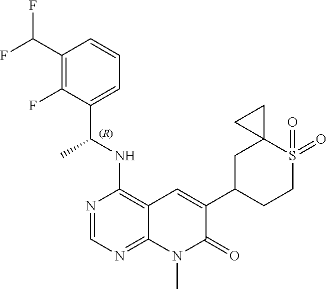

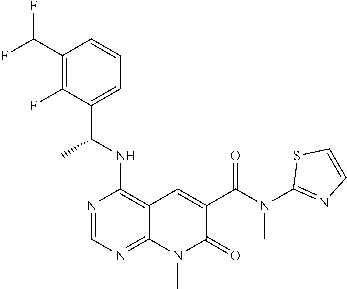 [SMILES: C[C@@H](Nc1ncnc2c1cc(C(=O)N(C)c1nccs1)c(=O)n2C)c1cccc(C(F)F)c1F]

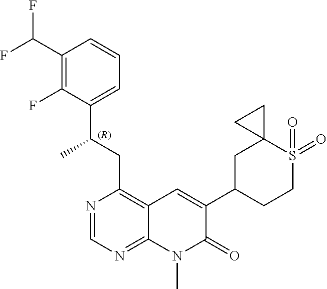

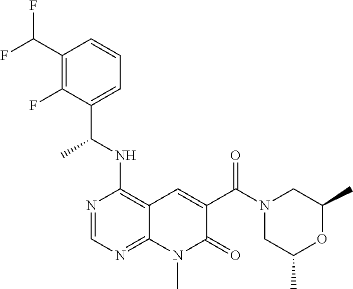 [SMILES: C[C@@H]1CN(C(=O)c2cc3c(N[C@H](C)c4cccc(C(F)F)c4F)ncnc3n(C)c2=O)C[C@@H](C)O1]

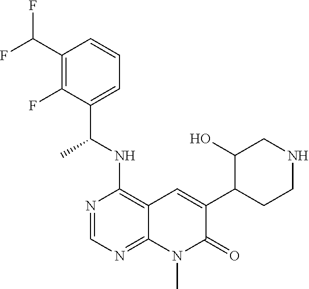 [SMILES: C[C@@H](Nc1ncnc2c1cc(C1CCNCC1O)c(=O)n2C)c1cccc(C(F)F)c1F]

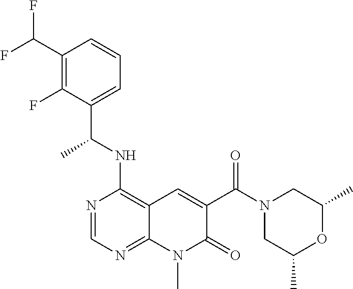 [SMILES: C[C@@H]1CN(C(=O)c2cc3c(N[C@H](C)c4cccc(C(F)F)c4F)ncnc3n(C)c2=O)C[C@H](C)O1]

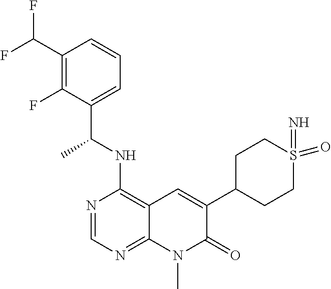 [SMILES: C[C@@H](Nc1ncnc2c1cc(C1CCS(=N)(=O)CC1)c(=O)n2C)c1cccc(C(F)F)c1F]

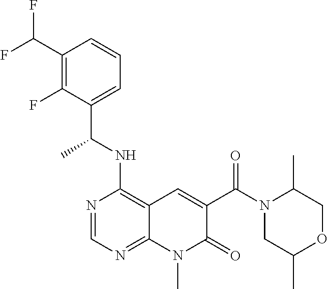 [SMILES: CC1CN(C(=O)c2cc3c(N[C@H](C)c4cccc(C(F)F)c4F)ncnc3n(C)c2=O)C(C)CO1]

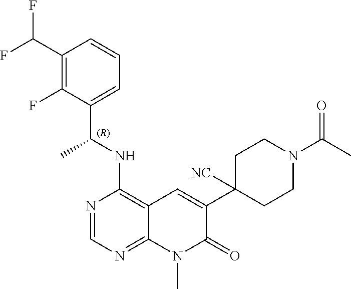

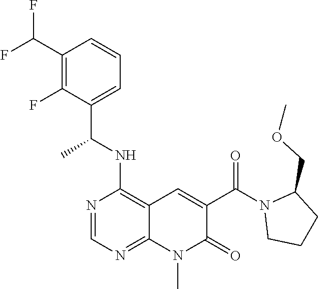 [SMILES: COC[C@H]1CCCN1C(=O)c1cc2c(N[C@H](C)c3cccc(C(F)F)c3F)ncnc2n(C)c1=O]

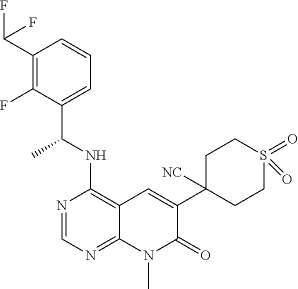 [SMILES: C[C@@H](Nc1ncnc2c1cc(C1(C#N)CCS(=O)(=O)CC1)c(=O)n2C)c1cccc(C(F)F)c1F]

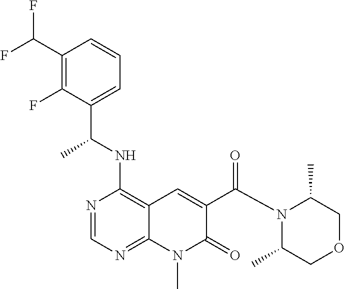 [SMILES: C[C@@H]1COC[C@H](C)N1C(=O)c1cc2c(N[C@H](C)c3cccc(C(F)F)c3F)ncnc2n(C)c1=O]

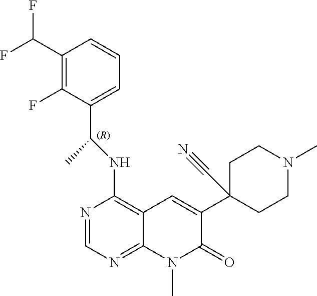

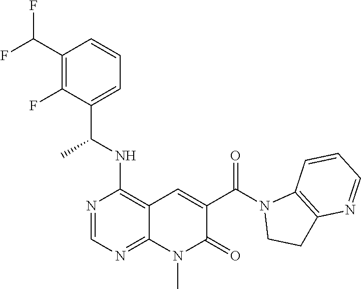 [SMILES: C[C@@H](Nc1ncnc2c1cc(C(=O)N1CCc3ncccc31)c(=O)n2C)c1cccc(C(F)F)c1F]

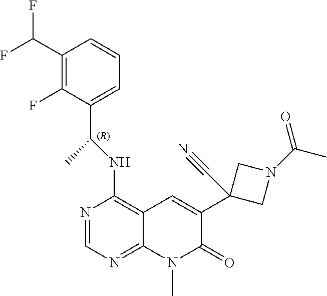

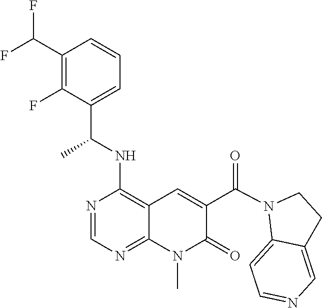 [SMILES: C[C@@H](Nc1ncnc2c1cc(C(=O)N1CCc3cnccc31)c(=O)n2C)c1cccc(C(F)F)c1F]

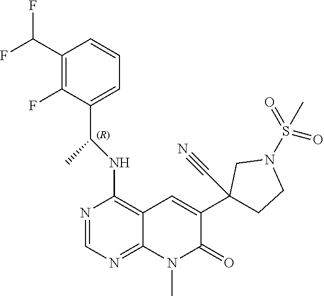

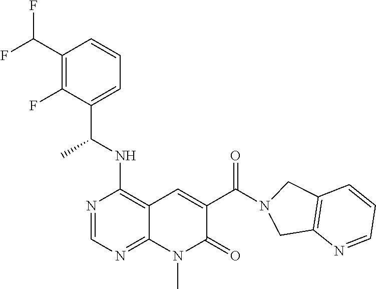 [SMILES: C[C@@H](Nc1ncnc2c1cc(C(=O)N1Cc3cccnc3C1)c(=O)n2C)c1cccc(C(F)F)c1F]

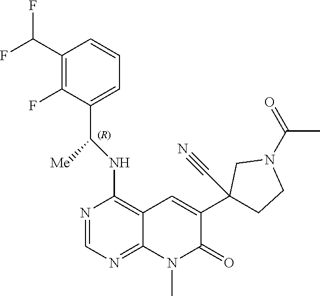

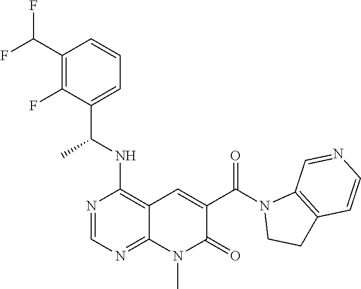 [SMILES: C[C@@H](Nc1ncnc2c1cc(C(=O)N1CCc3ccncc31)c(=O)n2C)c1cccc(C(F)F)c1F]

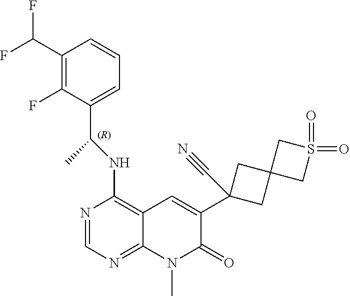

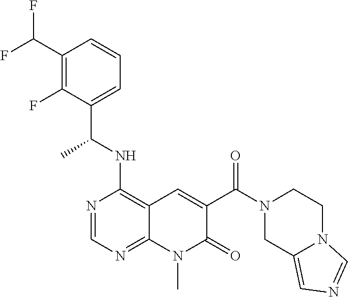 [SMILES: C[C@@H](Nc1ncnc2c1cc(C(=O)N1CCn3cncc3C1)c(=O)n2C)c1cccc(C(F)F)c1F]

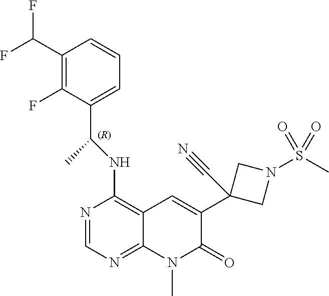

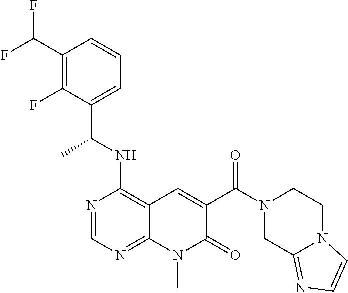 [SMILES: C[C@@H](Nc1ncnc2c1cc(C(=O)N1CCn3ccnc3C1)c(=O)n2C)c1cccc(C(F)F)c1F]

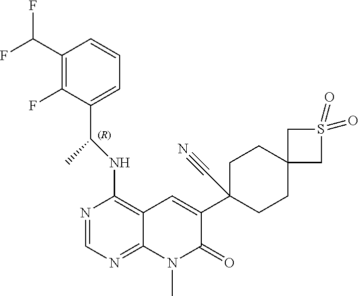

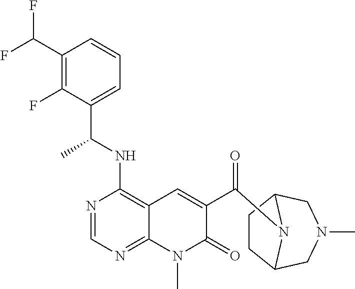 [SMILES: C[C@@H](Nc1ncnc2c1cc(C(=O)N1C3CCC1CN(C)C3)c(=O)n2C)c1cccc(C(F)F)c1F]

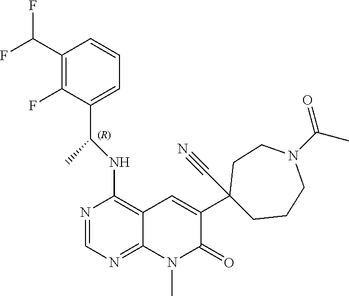

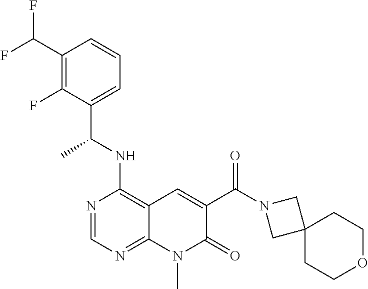 [SMILES: C[C@@H](Nc1ncnc2c1cc(C(=O)N1CC3(CCOCC3)C1)c(=O)n2C)c1cccc(C(F)F)c1F]

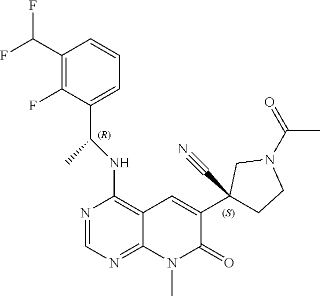

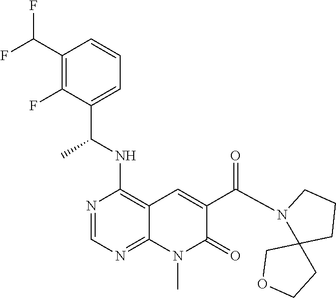 [SMILES: C[C@@H](Nc1ncnc2c1cc(C(=O)N1CCCC13CCOC3)c(=O)n2C)c1cccc(C(F)F)c1F]

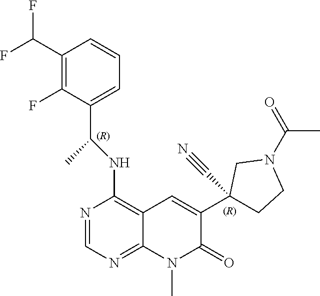

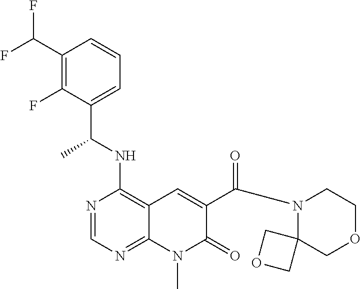 [SMILES: C[C@@H](Nc1ncnc2c1cc(C(=O)N1CCOCC13COC3)c(=O)n2C)c1cccc(C(F)F)c1F]

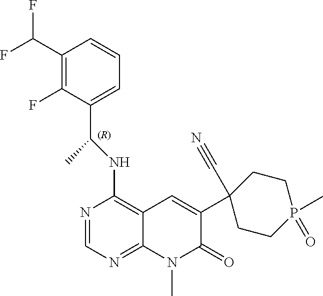

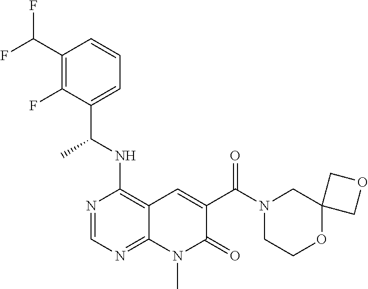 [SMILES: C[C@@H](Nc1ncnc2c1cc(C(=O)N1CCOC3(COC3)C1)c(=O)n2C)c1cccc(C(F)F)c1F]

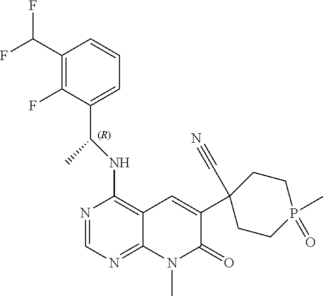

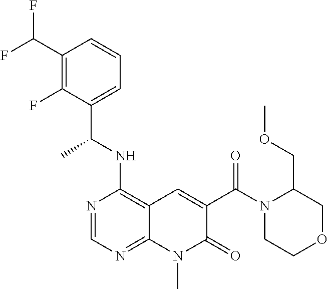 [SMILES: COCC1COCCN1C(=O)c1cc2c(N[C@H](C)c3cccc(C(F)F)c3F)ncnc2n(C)c1=O]

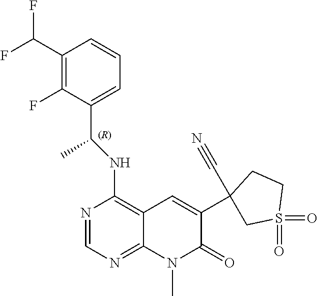

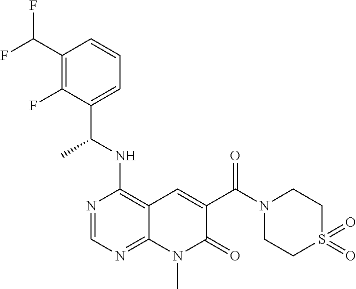 [SMILES: C[C@@H](Nc1ncnc2c1cc(C(=O)N1CCS(=O)(=O)CC1)c(=O)n2C)c1cccc(C(F)F)c1F]

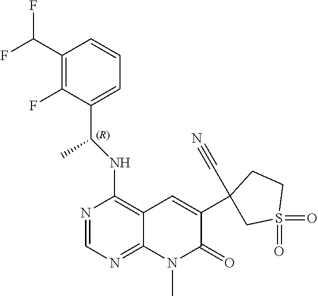

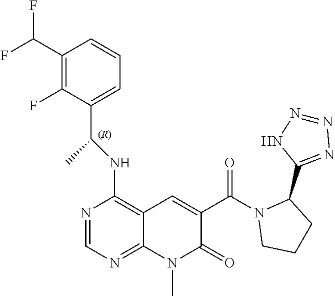

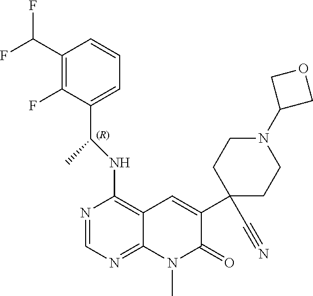

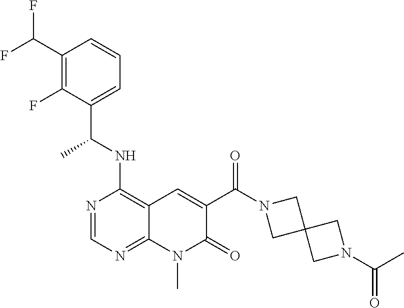 [SMILES: CC(=O)N1CC2(C1)CN(C(=O)c1cc3c(N[C@H](C)c4cccc(C(F)F)c4F)ncnc3n(C)c1=O)C2]

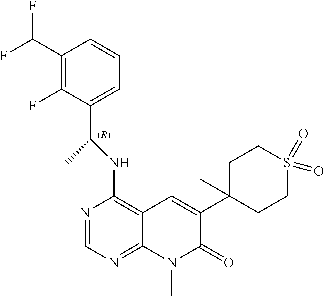

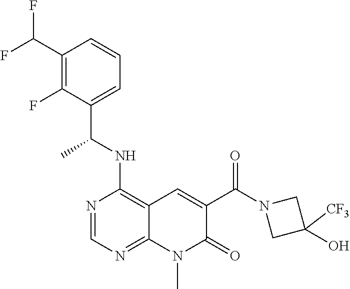 [SMILES: C[C@@H](Nc1ncnc2c1cc(C(=O)N1CC(O)(C(F)(F)F)C1)c(=O)n2C)c1cccc(C(F)F)c1F]

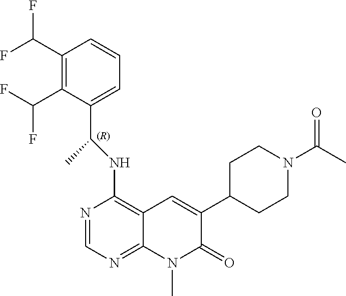

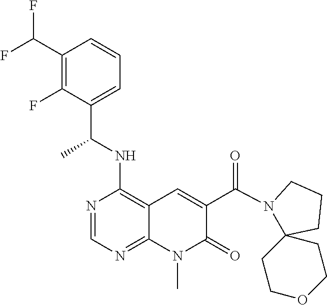 [SMILES: C[C@@H](Nc1ncnc2c1cc(C(=O)N1CCCC13CCOCC3)c(=O)n2C)c1cccc(C(F)F)c1F]

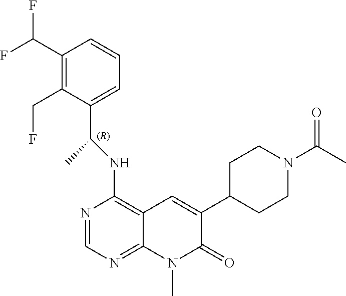

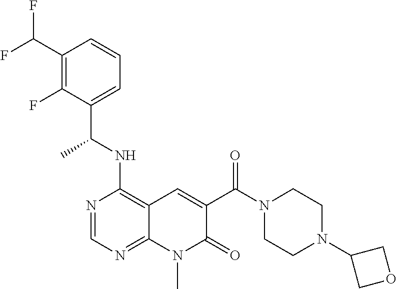 [SMILES: C[C@@H](Nc1ncnc2c1cc(C(=O)N1CCN(C3COC3)CC1)c(=O)n2C)c1cccc(C(F)F)c1F]

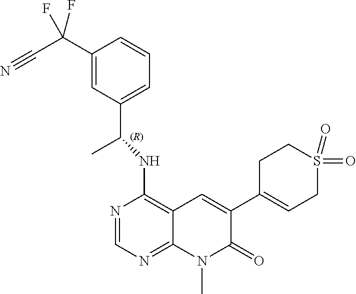

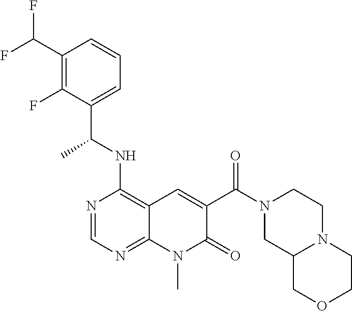 [SMILES: C[C@@H](Nc1ncnc2c1cc(C(=O)N1CCN3CCOCC3C1)c(=O)n2C)c1cccc(C(F)F)c1F]

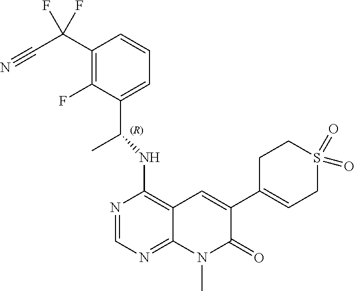

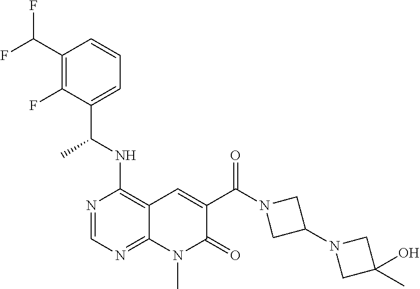 [SMILES: C[C@@H](Nc1ncnc2c1cc(C(=O)N1CC(N3CC(C)(O)C3)C1)c(=O)n2C)c1cccc(C(F)F)c1F]

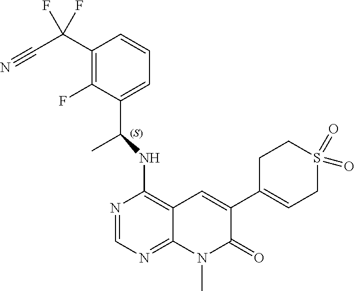

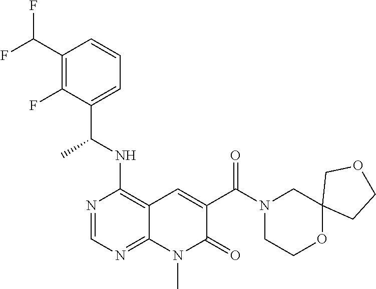 [SMILES: C[C@@H](Nc1ncnc2c1cc(C(=O)N1CCOC3(CCOC3)C1)c(=O)n2C)c1cccc(C(F)F)c1F]

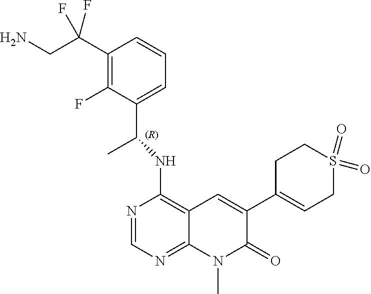

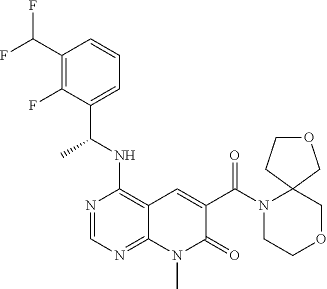 [SMILES: C[C@@H](Nc1ncnc2c1cc(C(=O)N1CCOCC13CCOC3)c(=O)n2C)c1cccc(C(F)F)c1F]

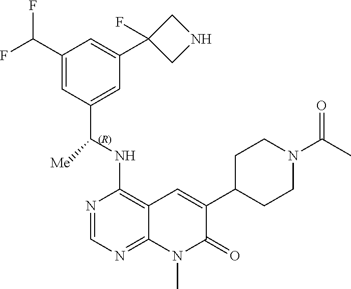

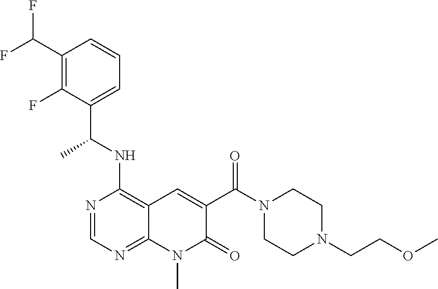 [SMILES: COCCN1CCN(C(=O)c2cc3c(N[C@H](C)c4cccc(C(F)F)c4F)ncnc3n(C)c2=O)CC1]

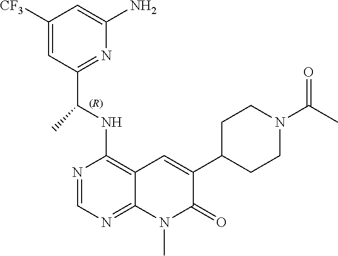

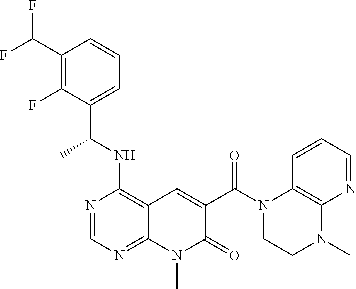 [SMILES: C[C@@H](Nc1ncnc2c1cc(C(=O)N1CCN(C)c3ncccc31)c(=O)n2C)c1cccc(C(F)F)c1F]

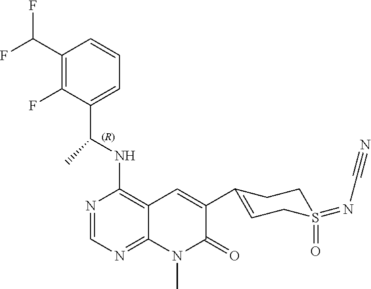

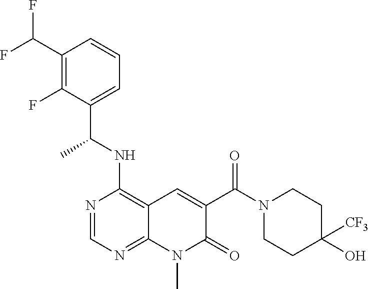 [SMILES: C[C@@H](Nc1ncnc2c1cc(C(=O)N1CCC(O)(C(F)(F)F)CC1)c(=O)n2C)c1cccc(C(F)F)c1F]

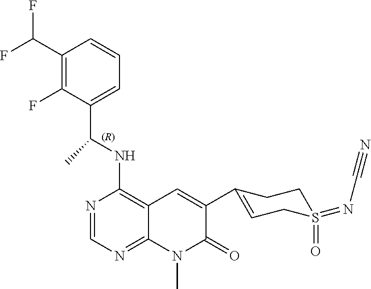

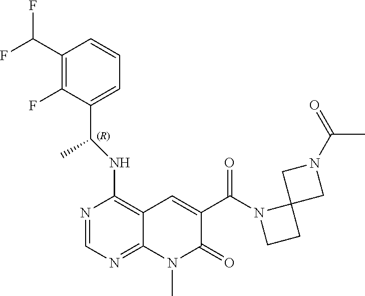

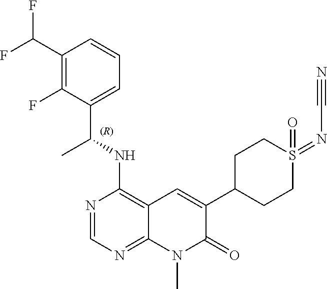

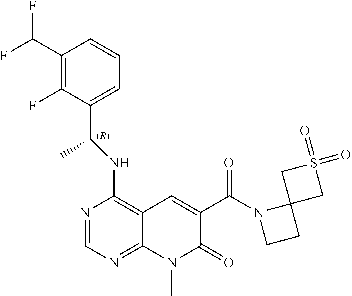

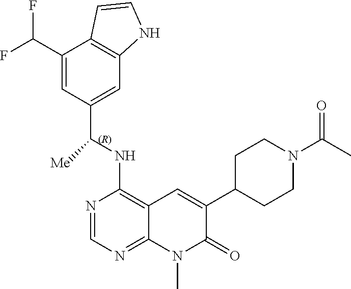

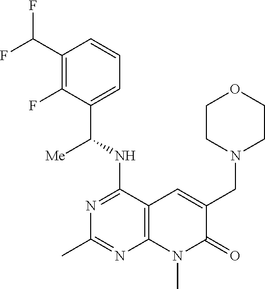 [SMILES: Cc1nc(N[C@H](C)c2cccc(C(F)F)c2F)c2cc(CN3CCOCC3)c(=O)n(C)c2n1]

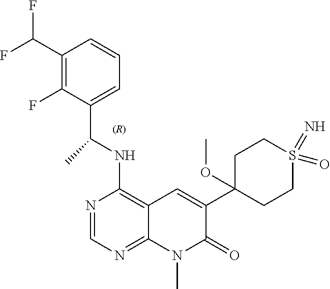

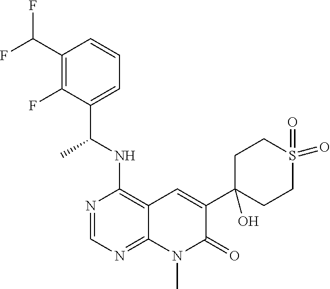 [SMILES: C[C@@H](Nc1ncnc2c1cc(C1(O)CCS(=O)(=O)CC1)c(=O)n2C)c1cccc(C(F)F)c1F]

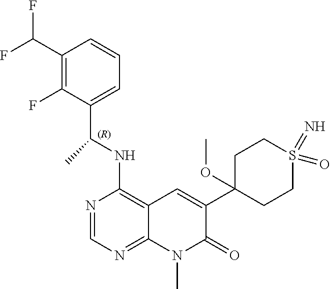

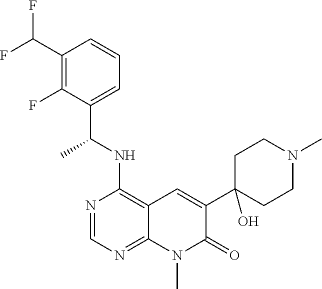 [SMILES: C[C@@H](Nc1ncnc2c1cc(C1(O)CCN(C)CC1)c(=O)n2C)c1cccc(C(F)F)c1F]

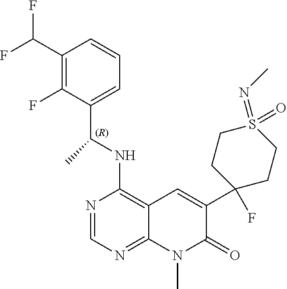

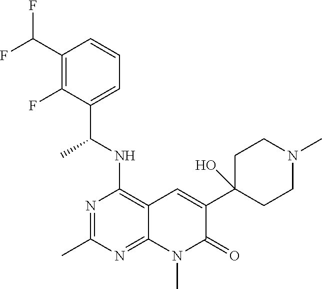 [SMILES: Cc1nc(N[C@H](C)c2cccc(C(F)F)c2F)c2cc(C3(O)CCN(C)CC3)c(=O)n(C)c2n1]

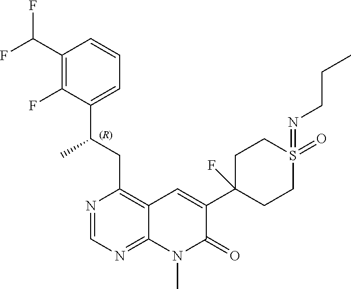

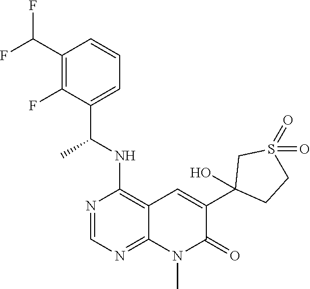 [SMILES: C[C@@H](Nc1ncnc2c1cc(C1(O)CCS(=O)(=O)C1)c(=O)n2C)c1cccc(C(F)F)c1F]

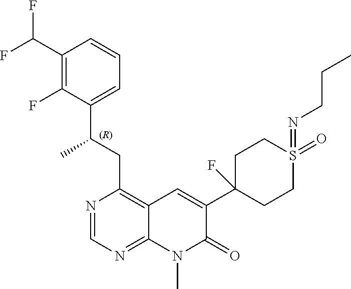

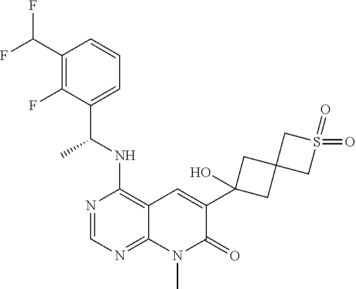 [SMILES: C[C@@H](Nc1ncnc2c1cc(C1(O)CC3(C1)CS(=O)(=O)C3)c(=O)n2C)c1cccc(C(F)F)c1F]

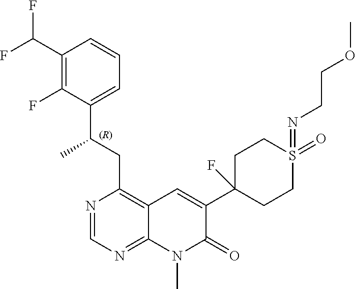

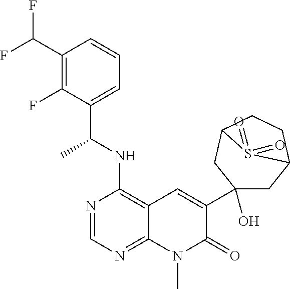 [SMILES: C[C@@H](Nc1ncnc2c1cc(C1(O)CC3CCC(C1)S3(=O)=O)c(=O)n2C)c1cccc(C(F)F)c1F]

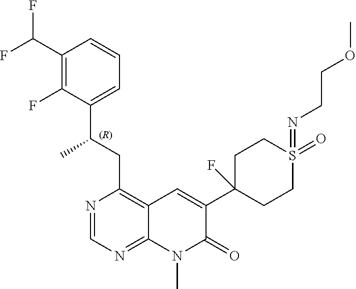

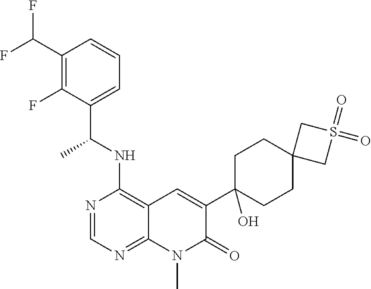 [SMILES: C[C@@H](Nc1ncnc2c1cc(C1(O)CCC3(CC1)CS(=O)(=O)C3)c(=O)n2C)c1cccc(C(F)F)c1F]

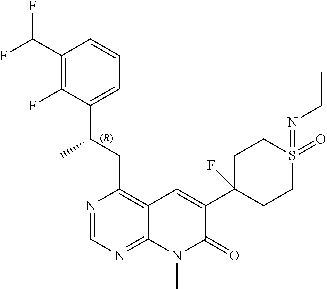

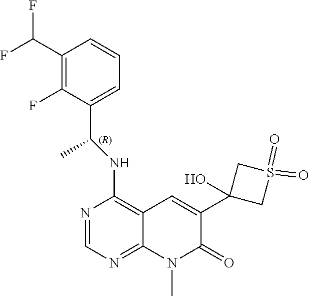

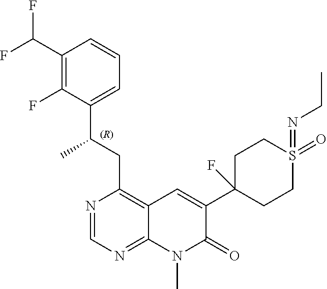

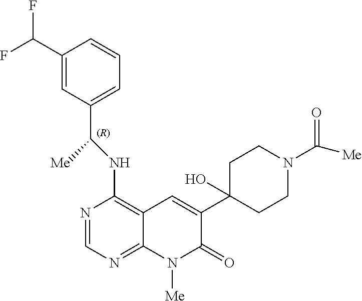

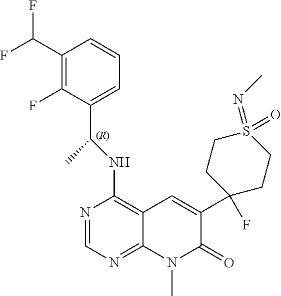

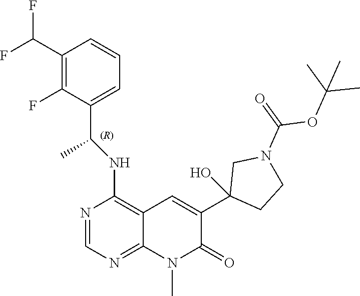

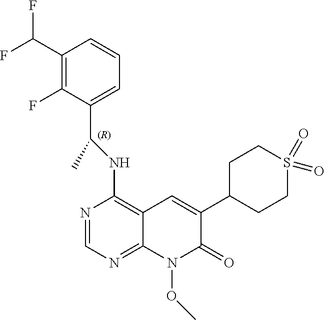

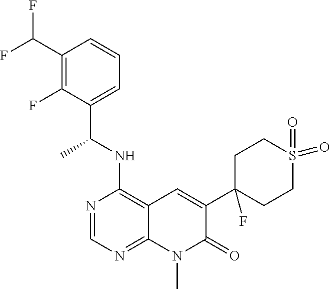 [SMILES: C[C@@H](Nc1ncnc2c1cc(C1(F)CCS(=O)(=O)CC1)c(=O)n2C)c1cccc(C(F)F)c1F]

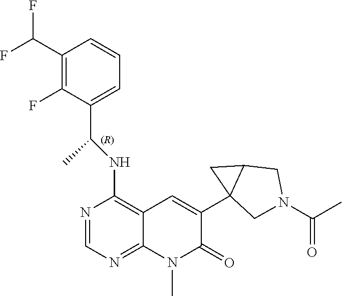

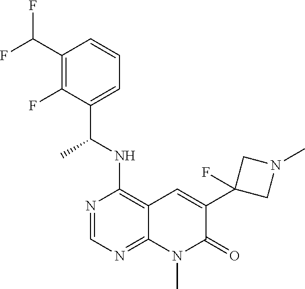 [SMILES: C[C@@H](Nc1ncnc2c1cc(C1(F)CN(C)C1)c(=O)n2C)c1cccc(C(F)F)c1F]

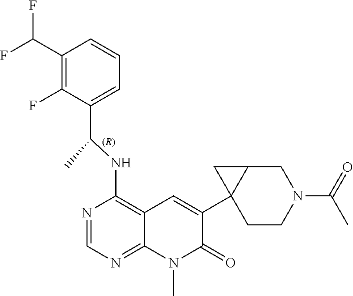

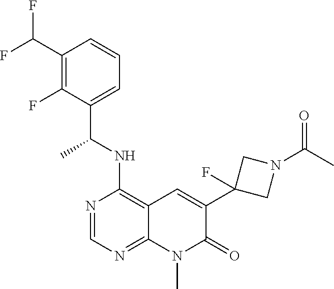 [SMILES: CC(=O)N1CC(F)(c2cc3c(N[C@H](C)c4cccc(C(F)F)c4F)ncnc3n(C)c2=O)C1]

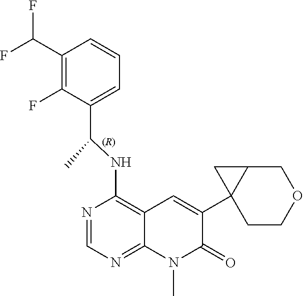

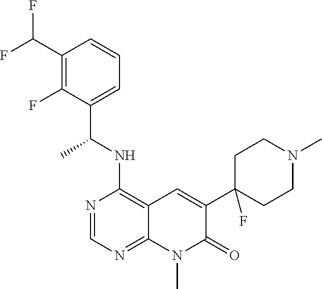 [SMILES: C[C@@H](Nc1ncnc2c1cc(C1(F)CCN(C)CC1)c(=O)n2C)c1cccc(C(F)F)c1F]

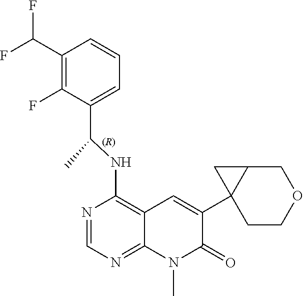

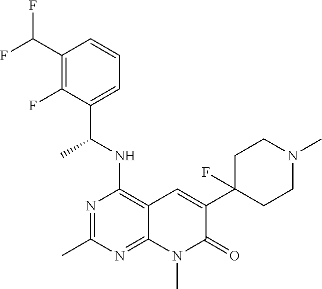 [SMILES: Cc1nc(N[C@H](C)c2cccc(C(F)F)c2F)c2cc(C3(F)CCN(C)CC3)c(=O)n(C)c2n1]

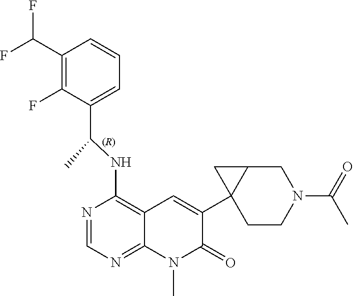

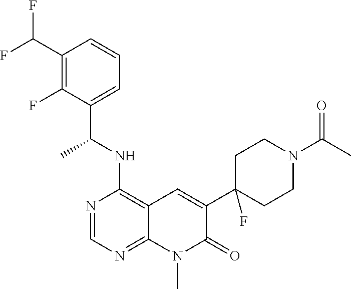 [SMILES: CC(=O)N1CCC(F)(c2cc3c(N[C@H](C)c4cccc(C(F)F)c4F)ncnc3n(C)c2=O)CC1]

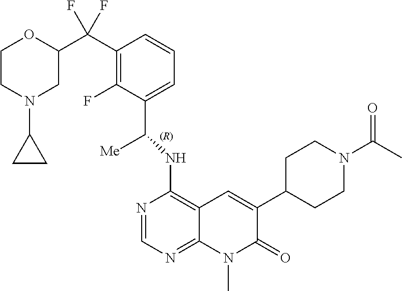

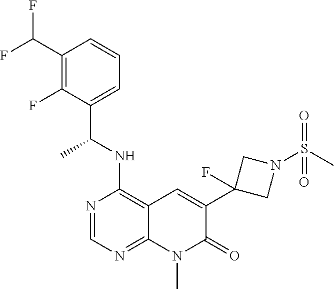 [SMILES: C[C@@H](Nc1ncnc2c1cc(C1(F)CN(S(C)(=O)=O)C1)c(=O)n2C)c1cccc(C(F)F)c1F]

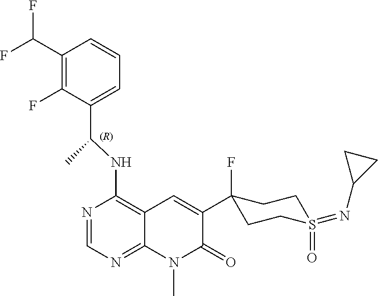

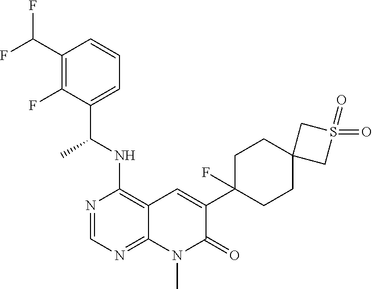 [SMILES: C[C@@H](Nc1ncnc2c1cc(C1(F)CCC3(CC1)CS(=O)(=O)C3)c(=O)n2C)c1cccc(C(F)F)c1F]

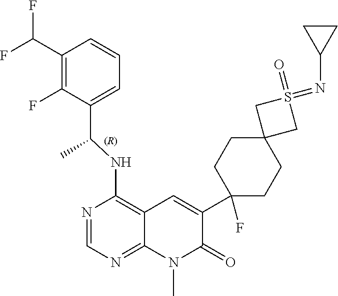

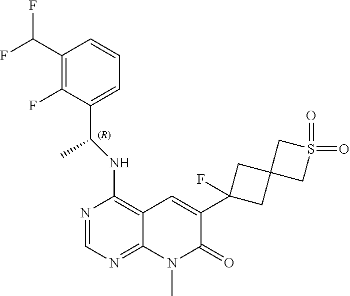

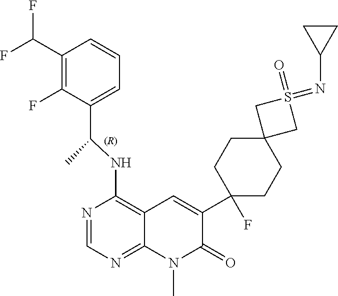

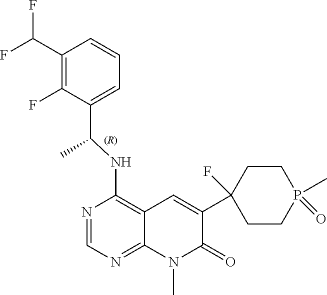

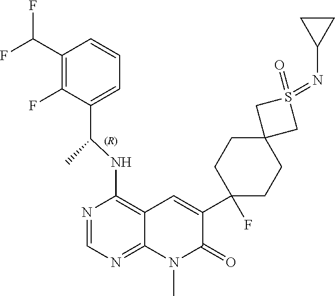

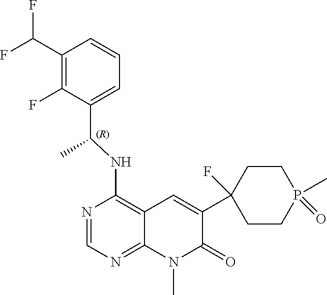

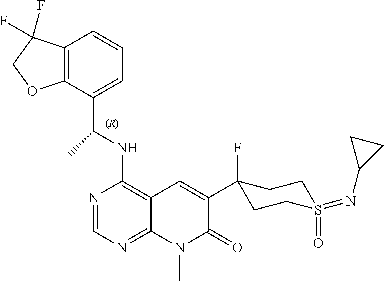

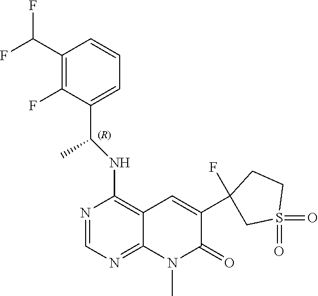

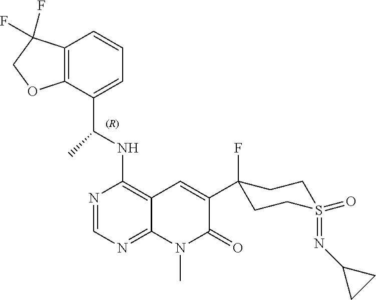

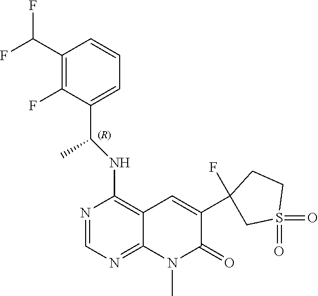

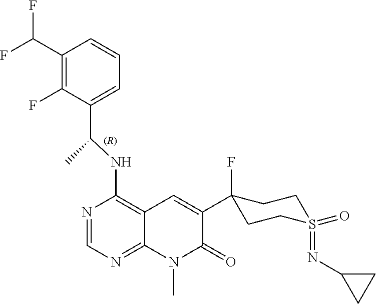

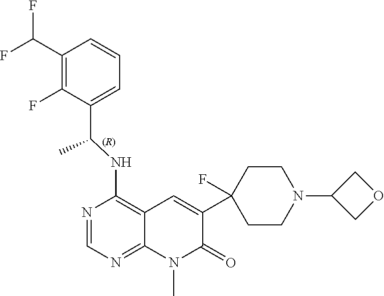

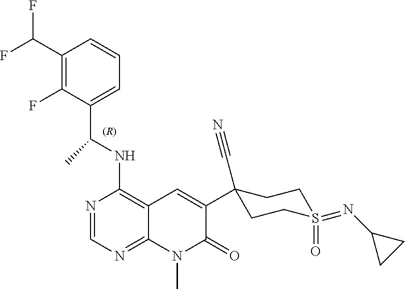

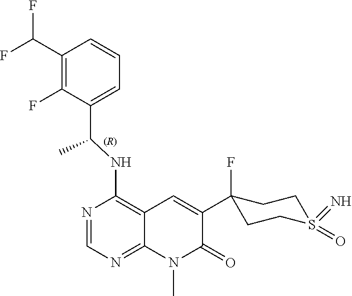

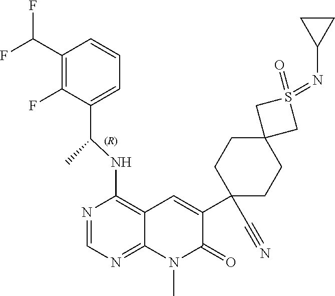

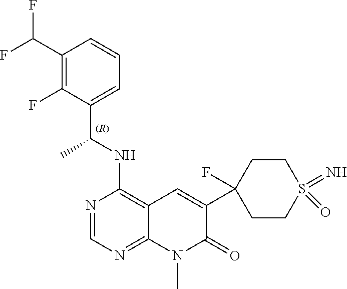

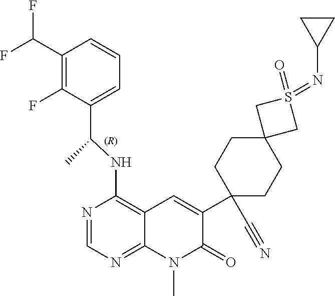

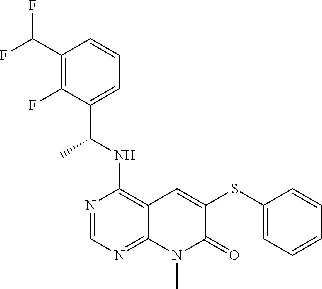 [SMILES: C[C@@H](Nc1ncnc2c1cc(Sc1ccccc1)c(=O)n2C)c1cccc(C(F)F)c1F]

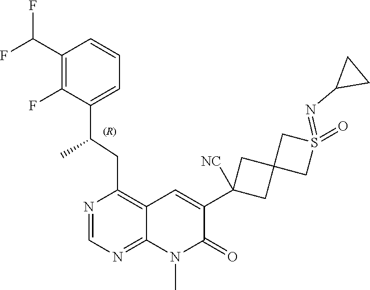

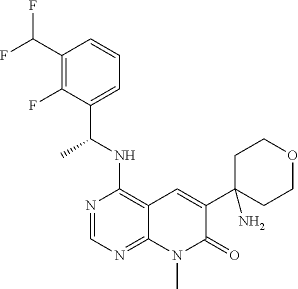 [SMILES: C[C@@H](Nc1ncnc2c1cc(C1(N)CCOCC1)c(=O)n2C)c1cccc(C(F)F)c1F]

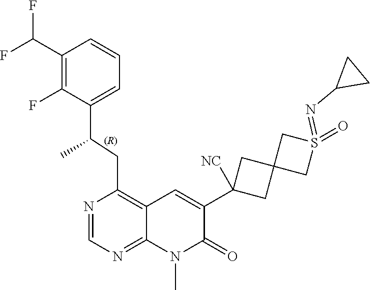

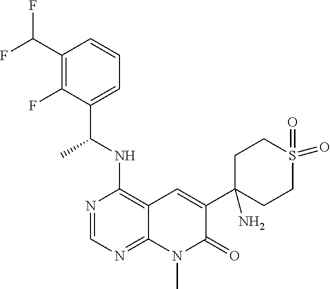 [SMILES: C[C@@H](Nc1ncnc2c1cc(C1(N)CCS(=O)(=O)CC1)c(=O)n2C)c1cccc(C(F)F)c1F]

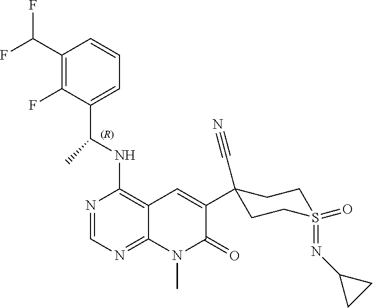

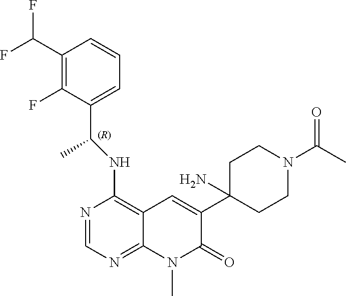

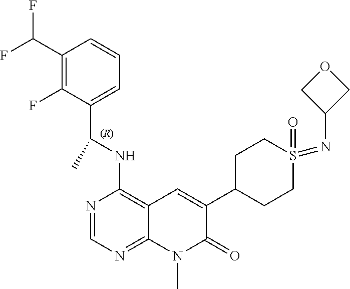

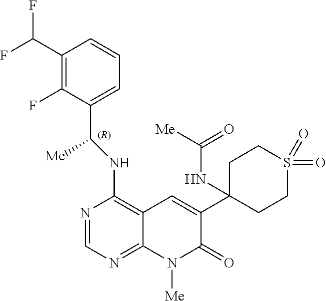

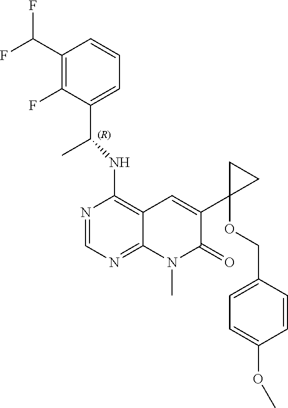

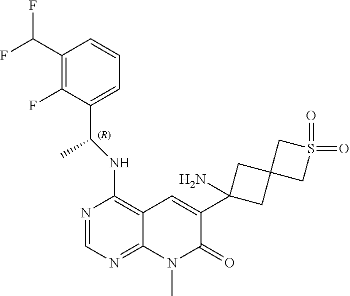

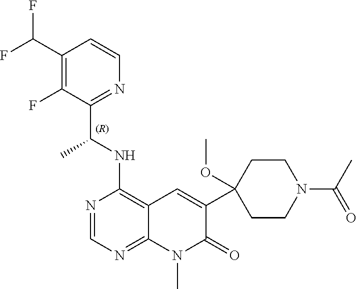

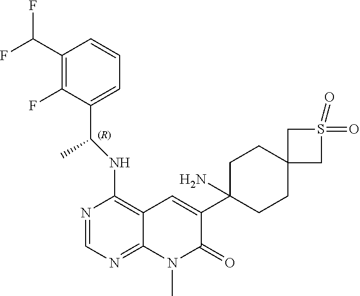

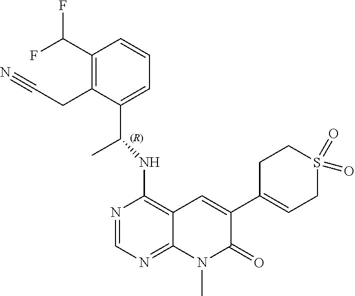

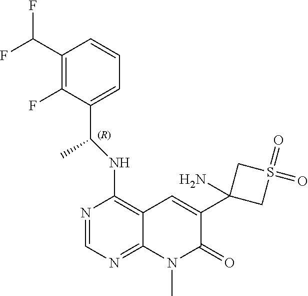

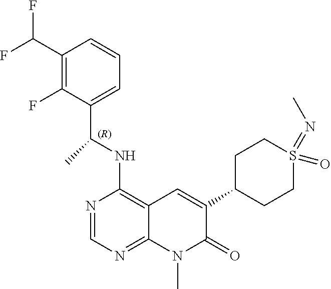

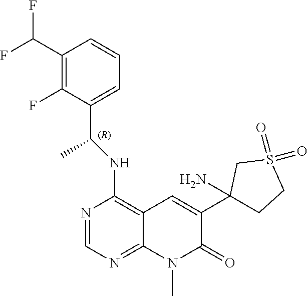

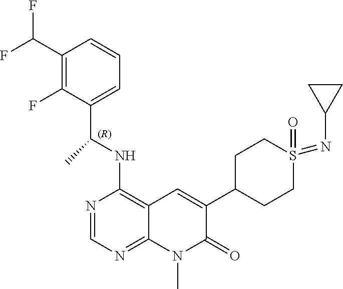

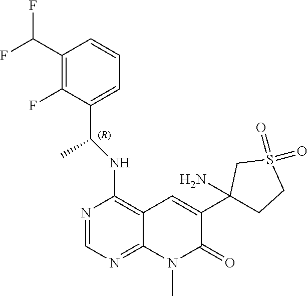

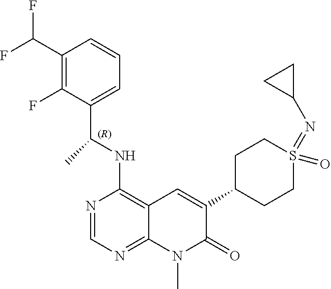

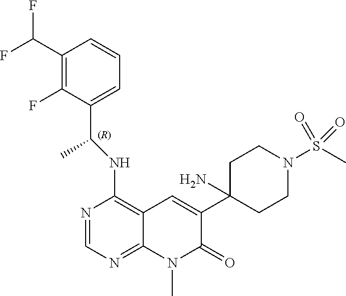

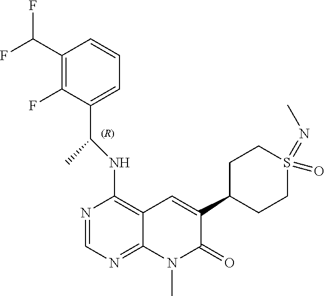

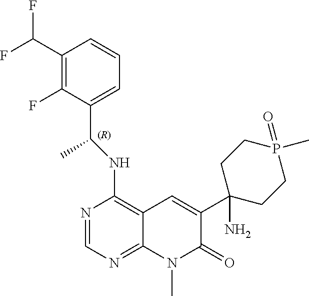

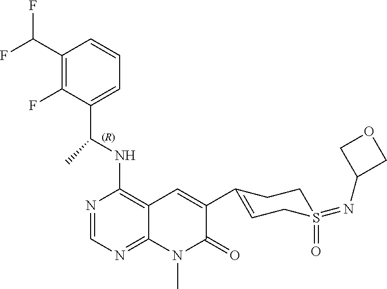

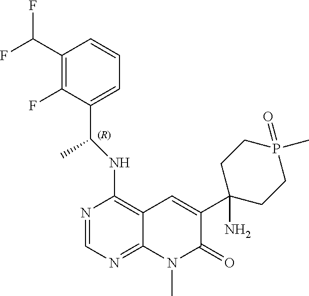

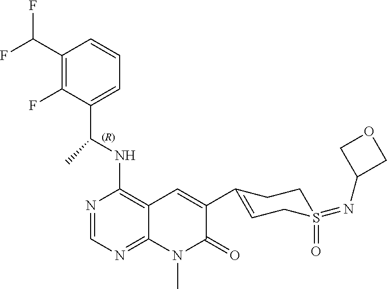

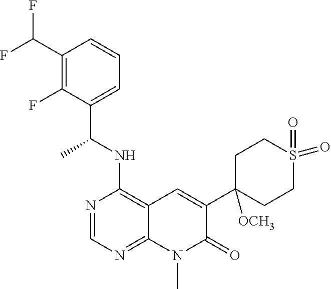 [SMILES: COC1(c2cc3c(N[C@H](C)c4cccc(C(F)F)c4F)ncnc3n(C)c2=O)CCS(=O)(=O)CC1]

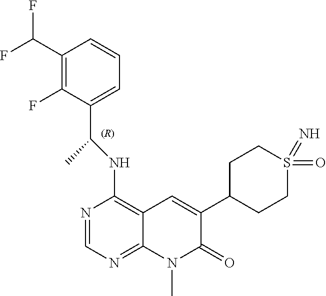

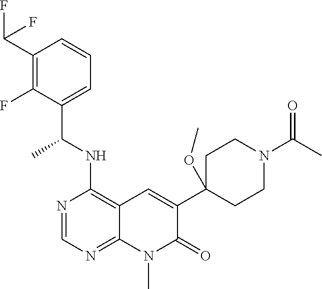 [SMILES: COC1(c2cc3c(N[C@H](C)c4cccc(C(F)F)c4F)ncnc3n(C)c2=O)CCN(C(C)=O)CC1]

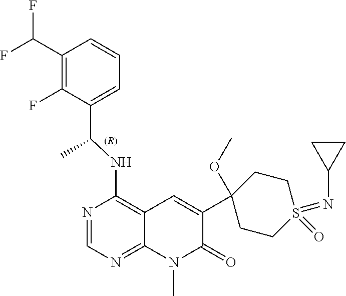

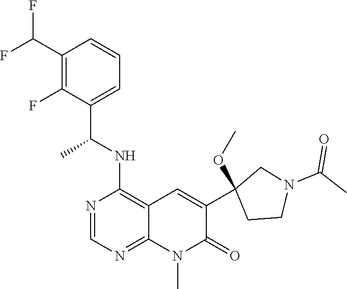 [SMILES: CO[C@]1(c2cc3c(N[C@H](C)c4cccc(C(F)F)c4F)ncnc3n(C)c2=O)CCN(C(C)=O)C1]

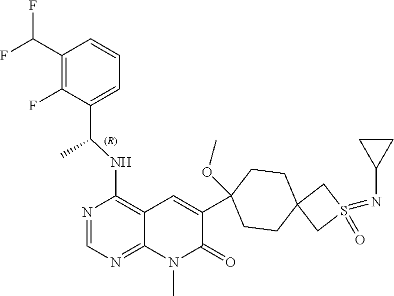

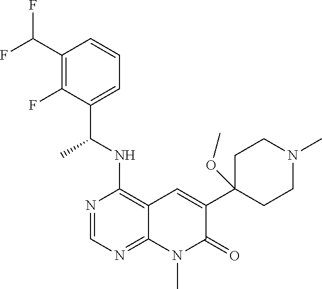 [SMILES: COC1(c2cc3c(N[C@H](C)c4cccc(C(F)F)c4F)ncnc3n(C)c2=O)CCN(C)CC1]

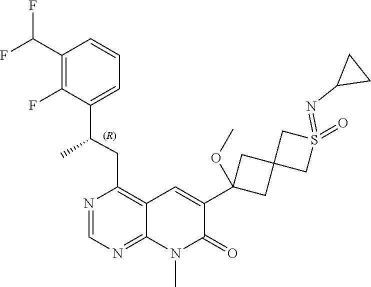

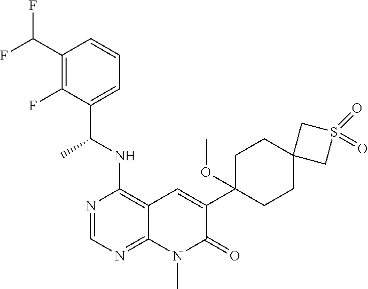 [SMILES: COC1(c2cc3c(N[C@H](C)c4cccc(C(F)F)c4F)ncnc3n(C)c2=O)CCC2(CC1)CS(=O)(=O)C2]

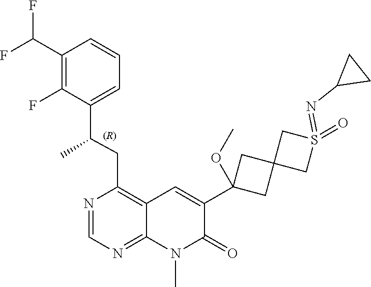

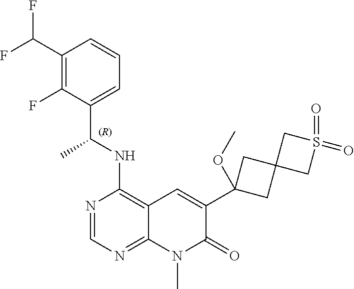

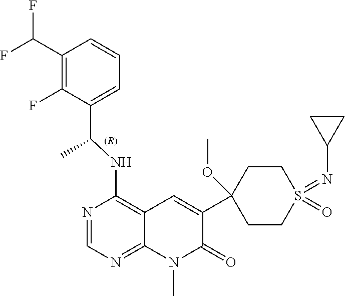

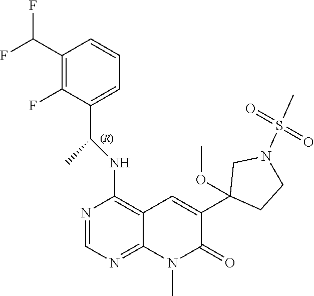

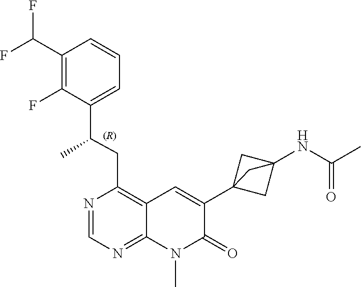

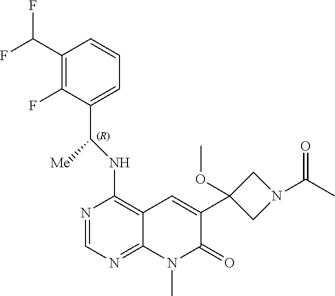

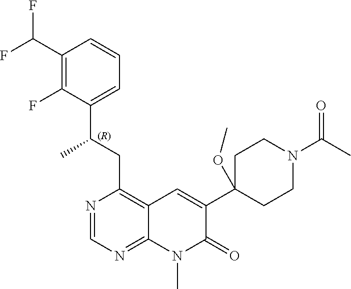

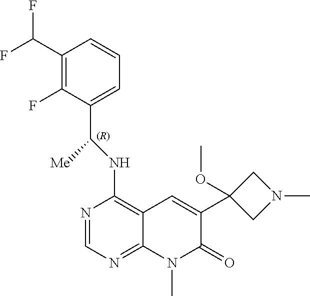

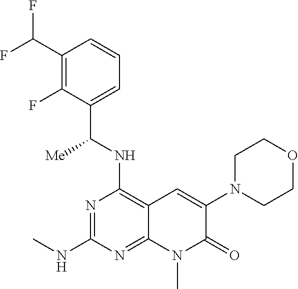 [SMILES: CNc1nc(N[C@H](C)c2cccc(C(F)F)c2F)c2cc(N3CCOCC3)c(=O)n(C)c2n1]

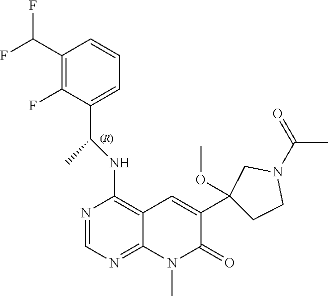

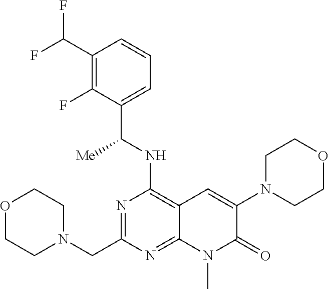 [SMILES: C[C@@H](Nc1nc(CN2CCOCC2)nc2c1cc(N1CCOCC1)c(=O)n2C)c1cccc(C(F)F)c1F]

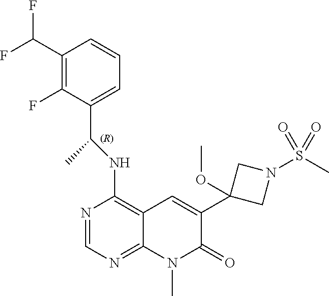

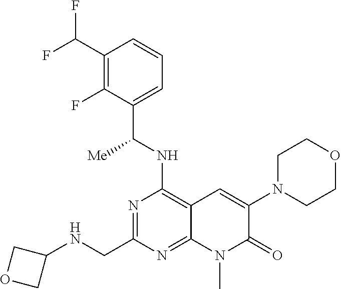 [SMILES: C[C@@H](Nc1nc(CNC2COC2)nc2c1cc(N1CCOCC1)c(=O)n2C)c1cccc(C(F)F)c1F]

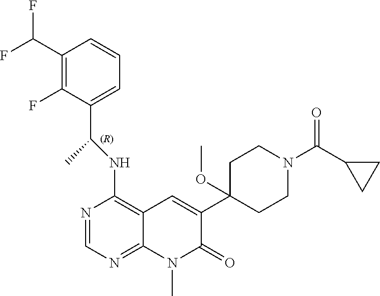

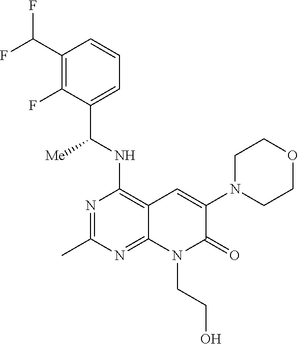 [SMILES: Cc1nc(N[C@H](C)c2cccc(C(F)F)c2F)c2cc(N3CCOCC3)c(=O)n(CCO)c2n1]

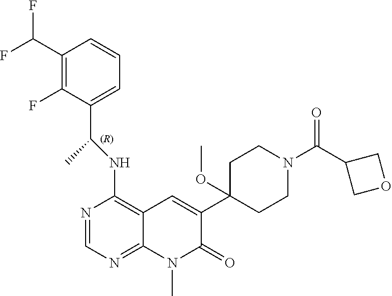

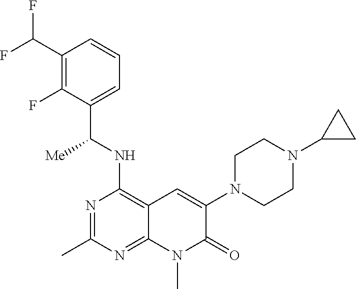 [SMILES: Cc1nc(N[C@H](C)c2cccc(C(F)F)c2F)c2cc(N3CCN(C4CC4)CC3)c(=O)n(C)c2n1]

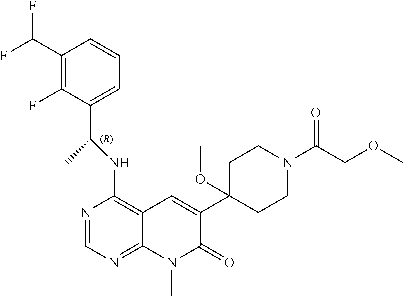

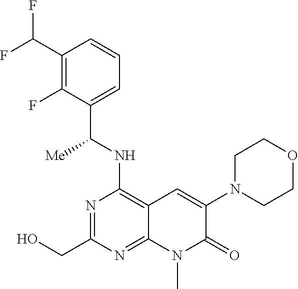 [SMILES: C[C@@H](Nc1nc(CO)nc2c1cc(N1CCOCC1)c(=O)n2C)c1cccc(C(F)F)c1F]

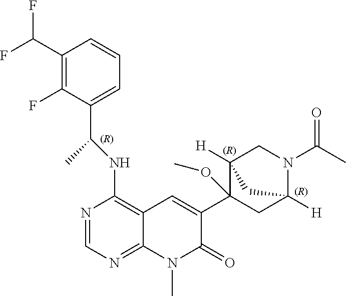

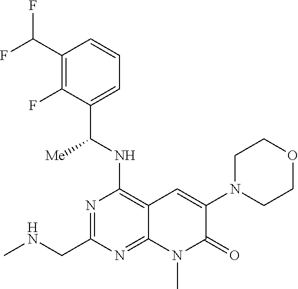 [SMILES: CNCc1nc(N[C@H](C)c2cccc(C(F)F)c2F)c2cc(N3CCOCC3)c(=O)n(C)c2n1]

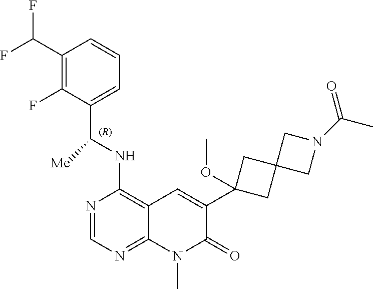

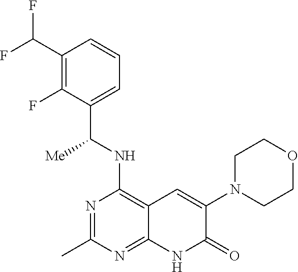 [SMILES: Cc1nc(N[C@H](C)c2cccc(C(F)F)c2F)c2cc(N3CCOCC3)c(=O)[nH]c2n1]

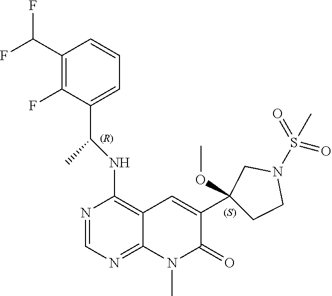

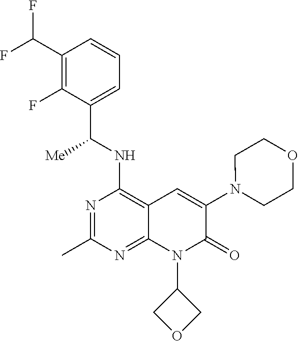 [SMILES: Cc1nc(N[C@H](C)c2cccc(C(F)F)c2F)c2cc(N3CCOCC3)c(=O)n(C3COC3)c2n1]

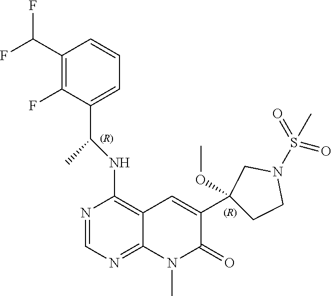

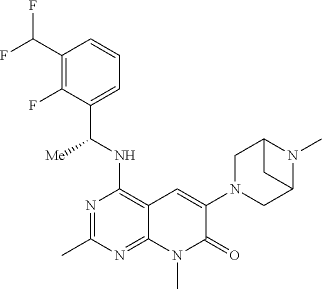 [SMILES: Cc1nc(N[C@H](C)c2cccc(C(F)F)c2F)c2cc(N3CC4CC(C3)N4C)c(=O)n(C)c2n1]

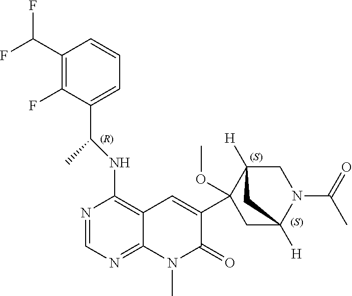

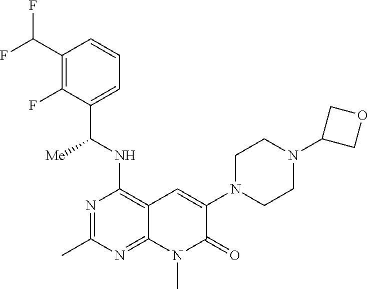 [SMILES: Cc1nc(N[C@H](C)c2cccc(C(F)F)c2F)c2cc(N3CCN(C4COC4)CC3)c(=O)n(C)c2n1]

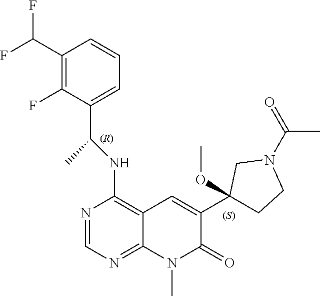

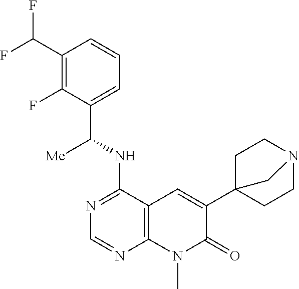 [SMILES: C[C@@H](Nc1ncnc2c1cc(C13CCN(CC1)C3)c(=O)n2C)c1cccc(C(F)F)c1F]

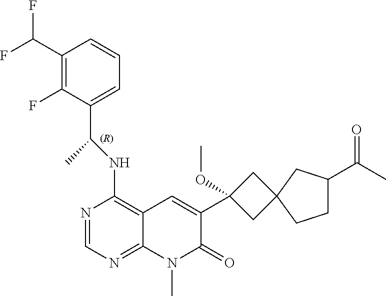

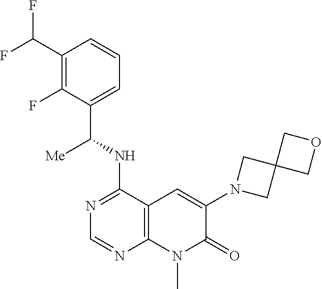 [SMILES: C[C@@H](Nc1ncnc2c1cc(N1CC3(COC3)C1)c(=O)n2C)c1cccc(C(F)F)c1F]

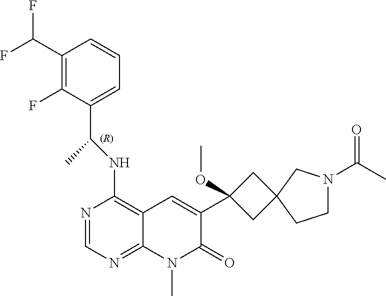

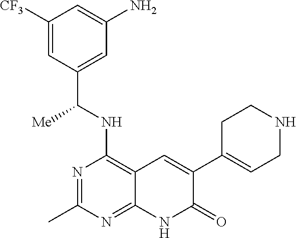 [SMILES: Cc1nc(N[C@H](C)c2cc(N)cc(C(F)(F)F)c2)c2cc(C3=CCNCC3)c(=O)[nH]c2n1]

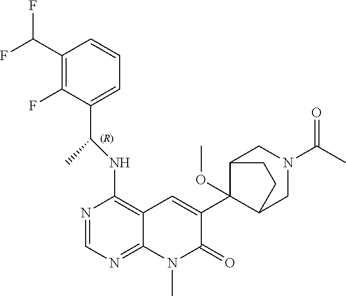

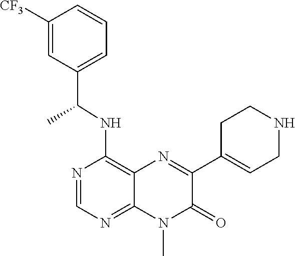 [SMILES: C[C@@H](Nc1ncnc2c1nc(C1=CCNCC1)c(=O)n2C)c1cccc(C(F)(F)F)c1]

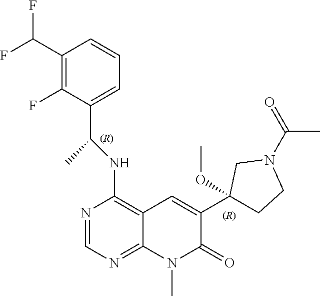

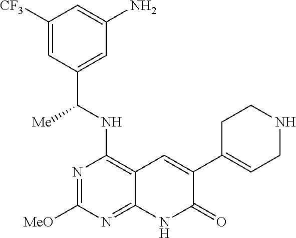 [SMILES: COc1nc(N[C@H](C)c2cc(N)cc(C(F)(F)F)c2)c2cc(C3=CCNCC3)c(=O)[nH]c2n1]

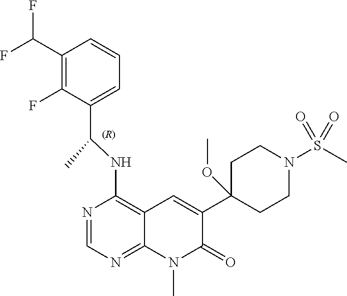

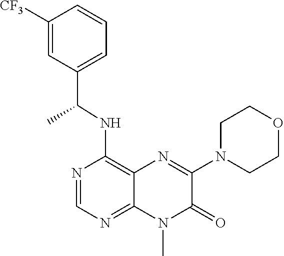 [SMILES: C[C@@H](Nc1ncnc2c1nc(N1CCOCC1)c(=O)n2C)c1cccc(C(F)(F)F)c1]

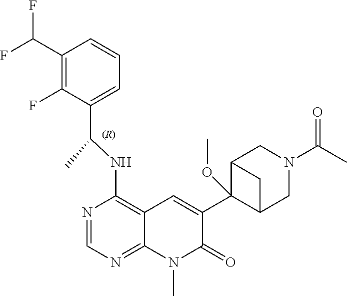

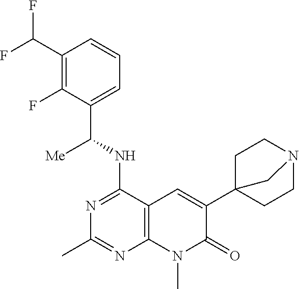 [SMILES: Cc1nc(N[C@H](C)c2cccc(C(F)F)c2F)c2cc(C34CCN(CC3)C4)c(=O)n(C)c2n1]

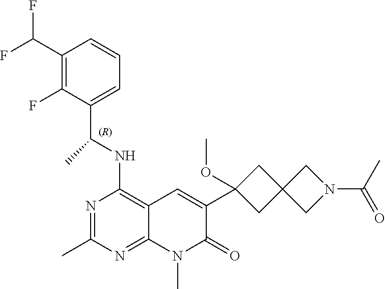

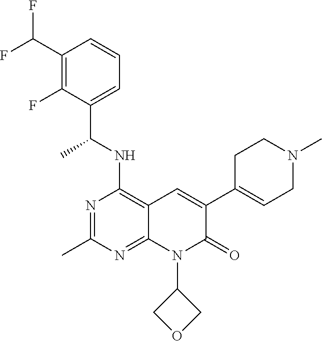 [SMILES: Cc1nc(N[C@H](C)c2cccc(C(F)F)c2F)c2cc(C3=CCN(C)CC3)c(=O)n(C3COC3)c2n1]

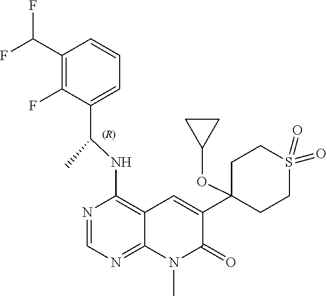

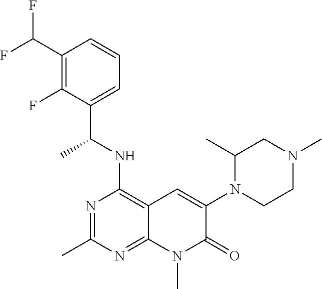 [SMILES: Cc1nc(N[C@H](C)c2cccc(C(F)F)c2F)c2cc(N3CCN(C)CC3C)c(=O)n(C)c2n1]

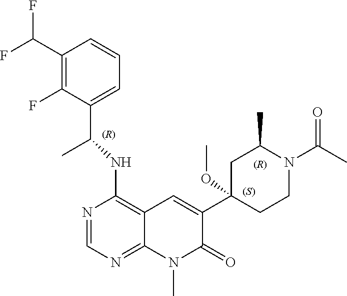

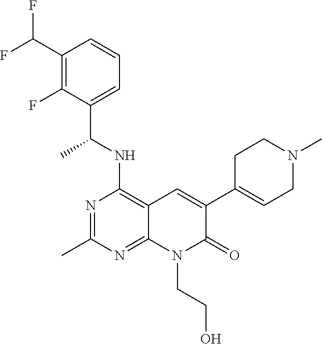 [SMILES: Cc1nc(N[C@H](C)c2cccc(C(F)F)c2F)c2cc(C3=CCN(C)CC3)c(=O)n(CCO)c2n1]

Note: any compound shown in the foregoing table may be a diastereomer, and the absolute stereochemistry of such diastereomer may not be known.

In some embodiments, the SOS1 inhibitor is selected from the group consisting of:
(R)-4-((1-(3-amino-5-(trifluoromethyl)phenyl) ethyl) amino)-8-methyl-6-(1,2,3,6-tetrahydropyridin-4-yl) pyrido[2,3-d]pyrimidin-7 (8H)-one;
(R)-4-((1-(3-(1,1-difluoro-2-hydroxyethyl)phenyl) ethyl) amino)-8-methyl-6-morpholinopyrido[2,3-d]pyrimidin-7 (8H)-one;
(R)-6-(3,6-dihydro-2H-pyran-4-yl)-8-methyl-4-((1-(3-(trifluoromethyl)phenyl) ethyl) amino)pyrido[2,3-d]pyrimidin-7 (8H)-one;
4-{[(1R)-1-[3-amino-5-(trifluoromethyl)phenyl]ethyl]amino}-8-methyl-6-(morpholin-4-yl)-7H,8H-pyrido[2,3-d]pyrimidin-7-one;
8-methyl-6-(morpholin-4-yl)-4-{[(1R)-1-[3-(trifluoromethyl)phenyl]-ethyl]amino}-7H,8H-pyrido[2,3-d]pyrimidin-7-one;
4-{[(1R)-1-[3-(difluoromethyl)-2-fluorophenyl]ethyl]amino}-6-(1-methanesulfonyl-3-methylazetidin-3-yl)-8-methyl-7H,8H-pyrido[2,3-d]pyrimidin-7-one;
6-(1-acetyl-4-piperidyl)-4-[(1R)-1-[3-amino-2-fluoro-5-(trifluoromethyl)phenyl]ethyl]amino]-8-methyl-pyrido[2,3-d]pyrimidin-7-one;
6-(1-acetyl-4-piperidyl)-4-[(1R)-1-[5-amino-2-fluoro-3-(trifluoromethyl)phenyl]ethyl]amino]-8-methyl-pyrido[2,3-d]pyrimidin-7-one;
(R)-4-((1-(3-(difluoromethyl)-2-fluorophenyl)ethyl) amino)-8-methyl-6-(pyridazin-4-yl) pyrido[2,3-d]pyrimidin-7 (8H)-one;
4-(((R)-1-(3-(difluoromethyl)-2-fluorophenyl)ethyl) amino)-8-methyl-6-(1-oxido-3,6-dihydro-2H-thiopyran-4-yl) pyrido[2,3-d]pyrimidin-7 (8H)-one;
4-(((R)-1-(3-(difluoromethyl)-2-fluorophenyl)ethyl) amino)-6-(1-imino-1-oxido-1,2,3,6-tetrahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl) amino)-8-methyl-6-(1-(methylimino)-1-oxido-1,2,3,6-tetrahydro-1λ⁶-thiopyran-4-yl) pyrido[2,3-d]pyrimidin-7 (8H)-one;
(R)-4-((1-(3-(difluoromethyl)-2-fluorophenyl)ethyl) amino)-8-methyl-6-(1-oxidotetrahydro-2H-thiopyran-4-yl) pyrido[2,3-d]pyrimidin-7 (8H)-one;
4-(4-{[(1R)-1-[3-(difluoromethyl)-2-fluorophenyl]-ethyl]amino}-8-methyl-7-oxo-7H,8H-pyrido[2,3-d]pyrimidin-6-yl)-4-hydroxy-1λ⁶-thiane-1,1-dione;
4-(4-{[(1R)-1-[3-(difluoromethyl)-2-fluorophenyl]ethyl]amino}-8-methyl-7-oxo-7H,8H-pyrido[2,3-d]-pyrimidin-6-yl)-4-fluoro-1λ⁶-thiane-1,1-dione;
4-{[(1R)-1-[3-(difluoromethyl)-2-fluorophenyl]ethyl]amino}-8-methyl-6-(phenylsulfanyl)-7H,8H-pyrido[2,3-d]pyrimidin-7-one;
6-(4-aminooxan-4-yl)-4-{[(1R)-1-[3-(difluoromethyl)-2-fluorophenyl]ethyl]amino}-8-methyl-7H,8H-pyrido-[2,3-d]pyrimidin-7-one;
4-(4-{[(1R)-1-[3-(difluoro-methyl)-2-fluorophenyl]ethyl]amino}-8-methyl-7-oxo-7H,8H-pyrido[2,3-d]pyrimidin-6-yl)-4-methoxy-1λ⁶-thiane-1,1-dione;
6-(4-{[(1R)-1-[3-(difluoromethyl)-2-fluorophenyl]ethyl]amino}-8-methyl-7-oxo-7H,8H-pyrido[2,3-d]pyrimi-din-6-yl)-226-thiaspiro[3.3]heptane-2,2-dione;
4-(((R)-1-(3-(difluoromethyl)-2-fluorophenyl)ethyl) amino)-6-(3-hydroxypiperidin-4-yl)-8-methylpyrido[2,3-d]pyrimidin-7 (8H)-one;
(R)-4-((1-(3-(difluoromethyl)-2-fluorophenyl)ethyl) amino)-6-(1-imino-1-oxidohexahydro-1λ⁶-thiopyran-4-yl)-8-methylpyrido[2,3-d]pyrimidin-7 (8H)-one;
1-acetyl-4-(4-{[(1R)-1-[3-(difluoromethyl)-2-fluorophenyl]ethyl]amino}-8-methyl-7-oxo-7H,8H-pyrido[2,3-d]pyrimidin-6-yl) piperidine-4-carbonitrile;
4-(4-{[(1R)-1-[3-(difluoromethyl)-2-fluorophenyl]ethyl]amino}-8-methyl-7-oxo-7H,8H-pyrido[2,3-d]pyrimidin-6-yl)-4-methyl-1λ⁶-thiane-1,1-dione;
6-(1-acetylpiperidin-4-yl)-4-{[(1R)-1-[2,3-bis (difluoromethyl)phenyl]ethyl]amino}-8-methyl-7H,8H-pyrido[2,3-d]pyrimidin-7-one;
6-(1-acetylpiperidin-4-yl)-4-{[(1R)-1-[3-(difluoromethyl)-2-(fluoromethyl)phenyl]ethyl]amino}-8-methyl-7H,8H-pyrido[2,3-d]pyrimidin-7-one;
2-{3-[(1R)-1-{[6-(1,1-dioxo-3,6-dihydro-2H-1λ⁶-thiopyran-4-yl)-8-methyl-7-oxo-7H,8H-pyrido[2,3-d]pyrimidin-4-yl]amino}ethyl]phenyl}-2,2-difluoroacetonitrile;
2-{3-[(1R)-1-{[6-(1,1-dioxo-3,6-dihydro-2H-16-thiopyran-4-yl)-8-methyl-7-oxo-7H,8H-pyrido[2,3-d]pyrimidin-4-yl]amino}ethyl]-2-fluorophenyl}-2,2-difluoroacetonitrile;
4-(4-{[(1R)-1-[3-(2-amino-1,1-difluoroethyl)-2-fluorophenyl]ethyl]amino}-8-methyl-7-oxo-7H,8H-pyrido[2,3-d]pyrimidin-6-yl)-3,6-dihydro-2H-1λ⁶-thiopyran-1,1-dione;
6-(1-acetyl-4-piperidyl)-4-[[(1R)-1-[3-(difluoromethyl)-5-(3-fluoroazetidin-3-yl)phenyl]ethyl]amino]-8-methyl-pyrido[2,3-d]pyrimidin-7-one;
[4-[4-[[(1R)-1-[3-(difluoromethyl)-2-fluoro-phenyl]ethyl]amino]-8-methyl-7-oxo-pyrido[2,3-d]pyrimidin-6-yl]-1-oxo-3,6-dihydro-2H-thiopyran-1-ylidene]cyanamide;
[4-[4-[(1R)-1-[3-(difluoromethyl)-2-fluoro-phenyl]ethyl]amino]-8-methyl-7-oxo-pyrido[2,3-d]pyrimidin-6-yl]-1-oxo-thian-1-ylidene]cyanamide;
4-(((R)-1-(3-(difluoromethyl)-2-fluorophenyl)ethyl) amino)-6-((1S,4s)-1-imino-4-methoxy-1-oxidohexahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl) amino)-6-((1R,4r)-1-imino-4-methoxy-1-oxidohexahydro-1λ⁶-thiopyran-4-yl)-8-methylpyrido[2,3-d]pyrimidin-7 (8H)-one;
4-(((R)-1-(3-(difluoromethyl)-2-fluorophenyl)ethyl) amino)-6-((1S,4s)-4-fluoro-1-(methylimino)-1-oxidohexahydro-16-thiopyran-4-yl)-8-methylpyrido[2,3-d]pyrimidin-7 (8H)-one;
4-(((R)-1-(3-(difluoromethyl)-2-fluorophenyl)ethyl) amino)-6-((1R,4r)-4-fluoro-1-(methylimino)-1-oxidohexahydro-1λ⁶-thiopyran-4-yl)-8-methylpyrido[2,3-d]pyrimidin-7 (8H)-one;
6-{3-acetyl-3-azabicyclo[3.1.0]hexan-1-yl}-4-{[(1R)-1-[3-(difluoromethyl)-2-fluorophenyl]ethyl]amino}-8-methyl-7H,8H-pyrido[2,3-d]pyrimidin-7-one;
6-(1-acetyl-4-piperidyl)-4-[(1R)-1-[3-[(4-cyclopropylmorpholin-2-yl)-difluoro-methyl]-2-fluoro-phenyl]ethyl]amino]-8-methyl-pyrido[2,3-d]pyrimidin-7-one;
6-((1R,4r)-1-(cyclopropylimino)-4-fluoro-1-oxidohexahydro-1λ⁶-thiopyran-4-yl)-4-(((R)-1-(3-(difluoromethyl)-2-fluorophenyl)ethyl) amino)-8-methylpyrido[2,3-d]pyrimidin-7 (8H)-one;
6-((1S,4s)-1-(cyclopropylimino)-4-fluoro-1-oxidohexahydro-1λ⁶-thiopyran-4-yl)-4-(((R)-1-(3-(difluoromethyl)-2-fluorophenyl)ethyl) amino)-8-methylpyrido[2,3-d]pyrimidin-7 (8H)-one;
(1R,4r)-1-(cyclopropylimino)-4-(4-(((R)-1-(3-(difluoromethyl)-2-fluorophenyl)ethyl) amino)-8-methyl-7-oxo-7,8-dihydropyrido[2,3-d]pyrimidin-6-yl) hexahydro-1λ⁶-thiopyran-4-carbonitrile 1-oxide;
(1S,4s)-1-(cyclopropylimino)-4-(4-(((R)-1-(3-(difluoromethyl)-2-fluorophenyl)ethyl) amino)-8-methyl-7-oxo-7,8-dihydropyrido[2,3-d]pyrimidin-6-yl) hexahydro-1λ⁶-thiopyran-4-carbonitrile 1-oxide;
4-[[(1R)-1-[3-(difluoromethyl)-2-fluoro-phenyl]ethyl]amino]-8-methyl-6-[1-(oxetan-3-ylimino)-1-oxo-thian-4-yl]pyrido[2,3-d]pyrimidin-7-one;
4-[(1R)-1-[3-(difluoromethyl)-2-fluoro-phenyl]ethyl]amino]-6-[1-[(4-methoxyphenyl) methoxy]cyclopropyl]-8-methyl-pyrido[2,3-d]pyrimidin-7-one;
2-[2-(difluoromethyl)-6-[(1R)-1-[[6-(1,1-dioxo-3,6-dihydro-2H-thiopyran-4-yl)-8-methyl-7-oxo-pyrido[2,3-d]pyrimidin-4-yl]amino]ethyl]phenyl]acetonitrile;
4-(((R)-1-(3-(difluoromethyl)-2-fluorophenyl)ethyl) amino)-8-methyl-6-((1R,4r)-1-(methylimino)-1-oxidohexahydro-1λ⁶-thiopyran-4-yl) pyrido[2,3-d]pyrimidin-7 (8H)-one;
4-(((R)-1-(3-(difluoromethyl)-2-fluorophenyl)ethyl) amino)-8-methyl-6-((S)-1-(oxetan-3-ylimino)-1-oxido-1,2,3,6-tetrahydro-1λ⁶-thiopyran-4-yl) pyrido[2,3-d]pyrimidin-7 (8H)-one;
4-(((R)-1-(3-(difluoromethyl)-2-fluorophenyl)ethyl) amino)-8-methyl-6-((R)-1-(oxetan-3-ylimino)-1-oxido-1,2,3,6-tetrahydro-1λ⁶-thiopyran-4-yl) pyrido[2,3-d]pyrimidin-7 (8H)-one;
4-[(1R)-1-[3-(difluoromethyl)-2-fluoro-phenyl]ethyl]amino]-6-(1-imino-1-oxo-thian-4-yl)-8-methyl-pyrido[2,3-d]pyrimidin-7-one;
6-((1R,4r)-1-(cyclopropylimino)-4-methoxy-1-oxidohexahydro-116-thiopyran-4-yl)-4-(((R)-1-(3-(difluoromethyl)-2-fluorophenyl)ethyl) amino)-8-methylpyrido [2,3-d]pyrimidin-7 (8H)-one;
6-((1S,4s)-1-(cyclopropylimino)-4-methoxy-1-oxidohexahydro-1λ⁶-thiopyran-4-yl)-4-(((R)-1-(3-(difluoromethyl)-2-fluorophenyl)ethyl) amino)-8-methylpyrido [2,3-d]pyrimidin-7 (8H)-one; and
N-[3-(4-{[(1R)-1-[3-(difluoromethyl)-2-fluorophenyl]ethyl]amino}-8-methyl-7-oxo-7H,8H-pyrido[2,3-d]pyrimidin-6-yl) bicyclo[1.1.1]pentan-1-yl]acetamide;
   or a pharmaceutically acceptable salt or a stereoisomer thereof.

In some embodiments, the SOS1 inhibitor is a compound selected from the group consisting of the compounds in the following table, or a pharmaceutically acceptable salt, solvate, isomer (e.g., stereoisomer), prodrug, or tautomer thereof, wherein any compound shown in brackets in the table indicates that the compound is a diastereomer, and the absolute stereochemistry of such diastereomer may not be known:

| Structure | Structure |
|---|---|
| 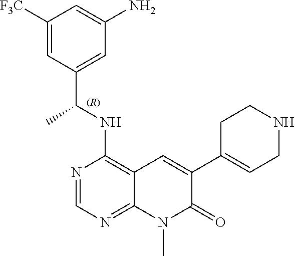 | 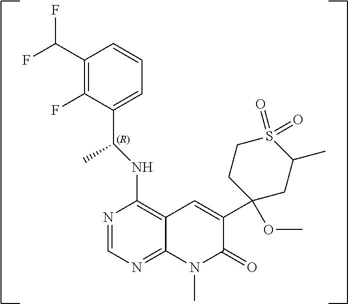 |
| 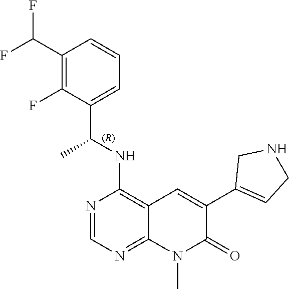 | 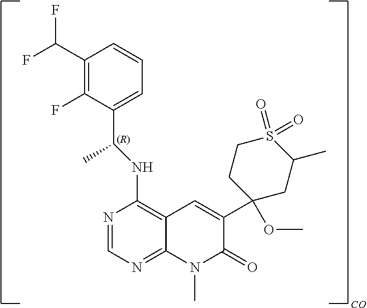 |
| 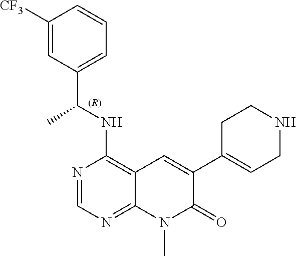 | 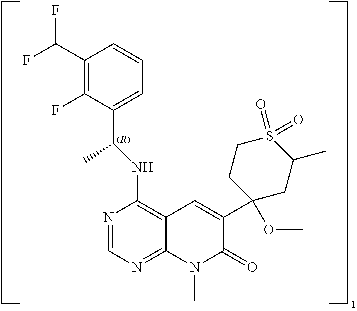 |
| 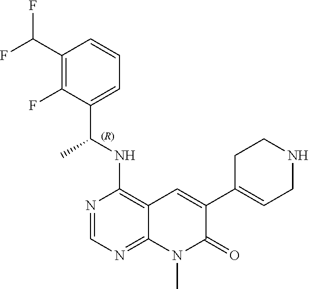 | 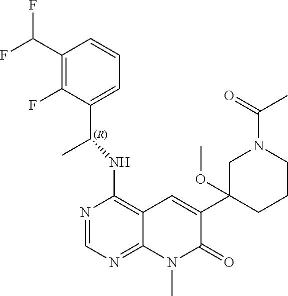 |
| 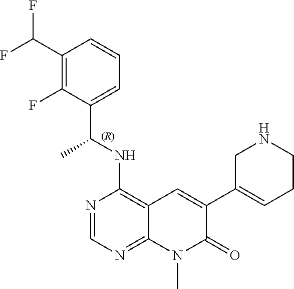 | 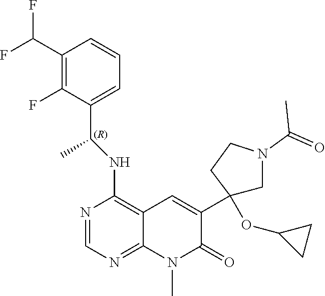 |
| 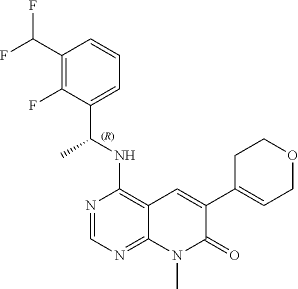 | 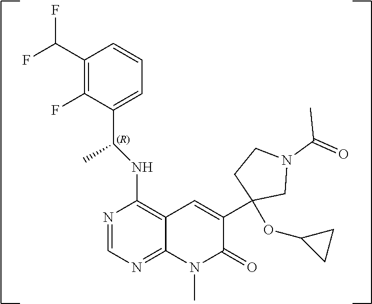 |
| 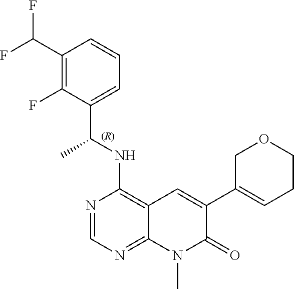 | 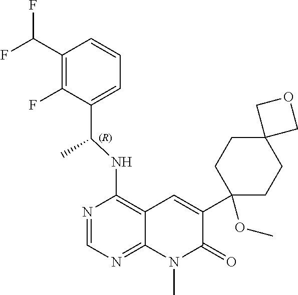 |
| 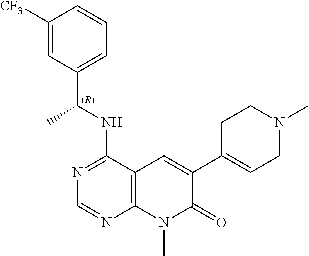 | 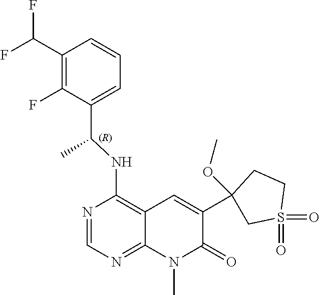 |
| 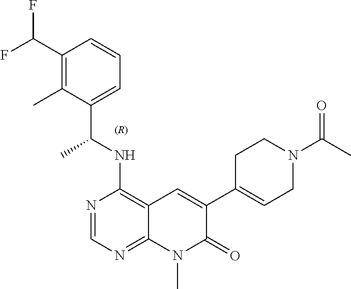 | 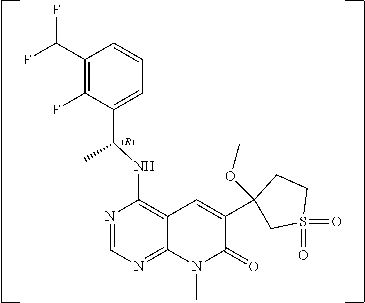 |
| 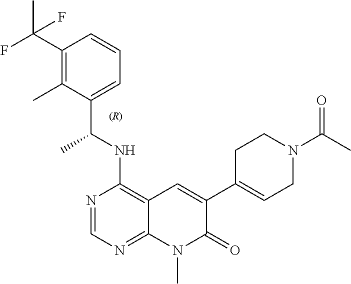 | 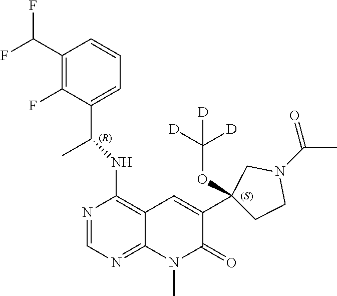 |
| 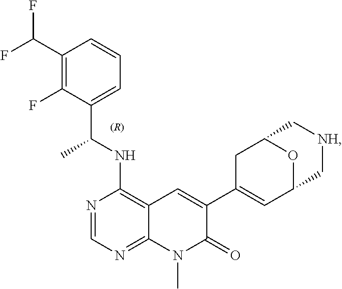 | 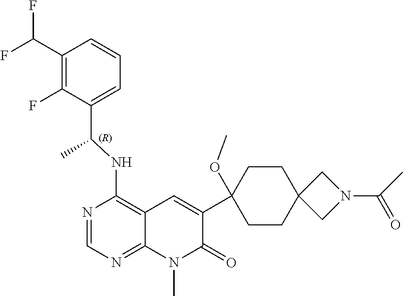 |
| 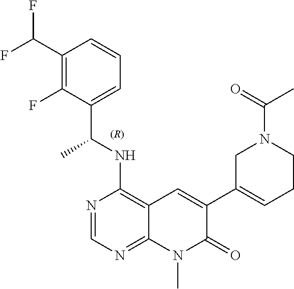 | 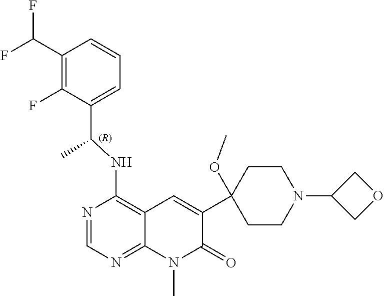 |
| 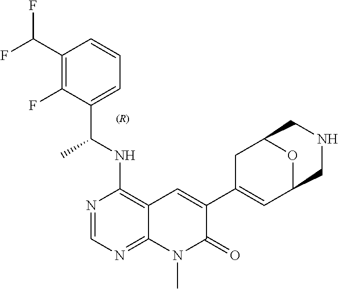 | 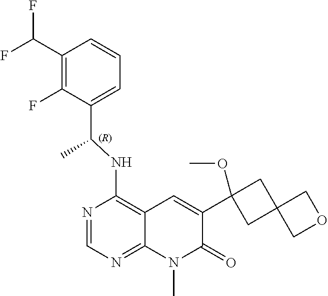 |
| 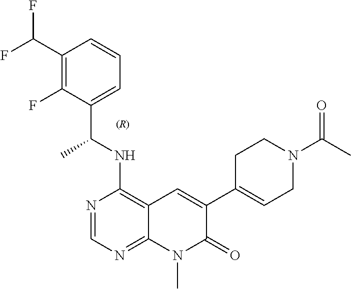 | 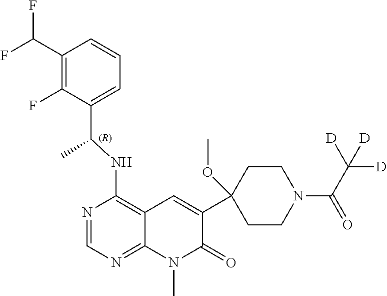 |
| 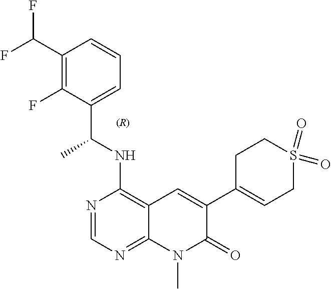 | 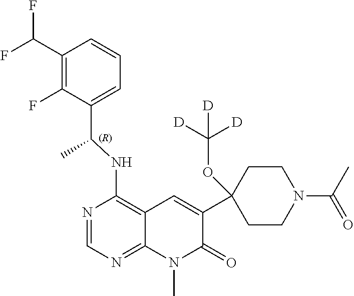 |
| 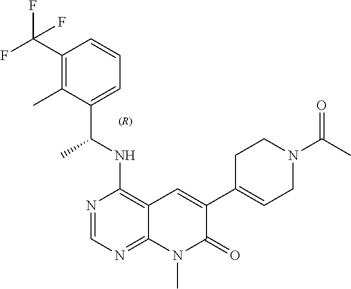 | 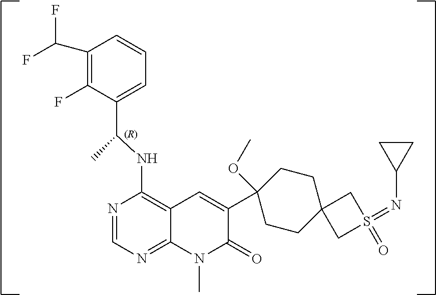 |
| 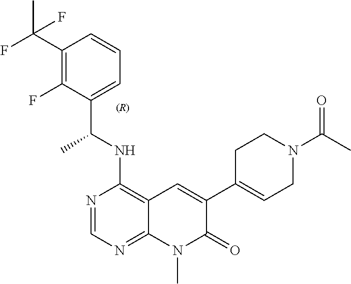 | 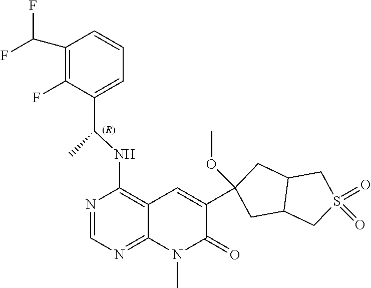 |
| 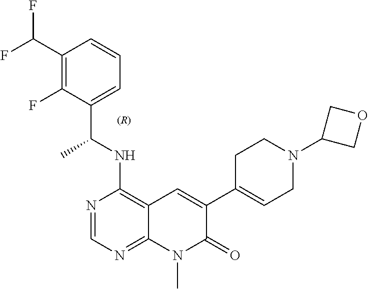 | 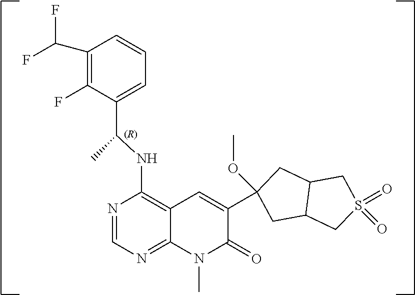 |
| 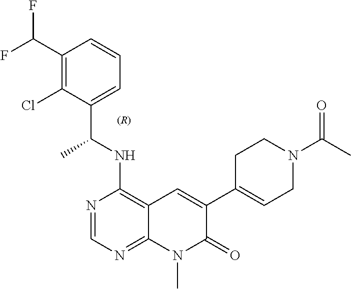 | 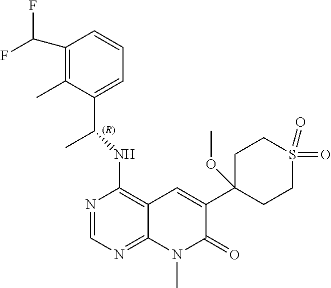 |
| 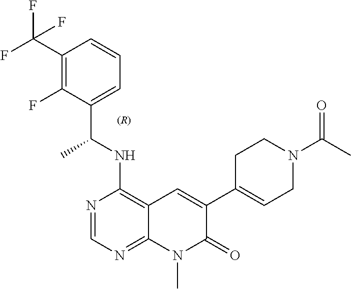 | 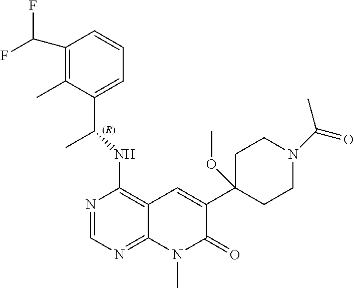 |
| 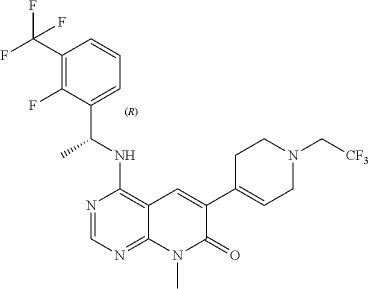 | 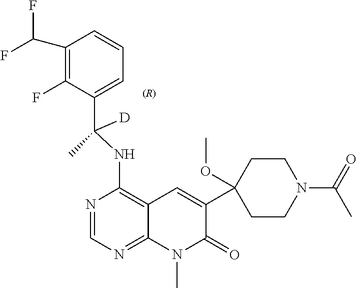 |
| 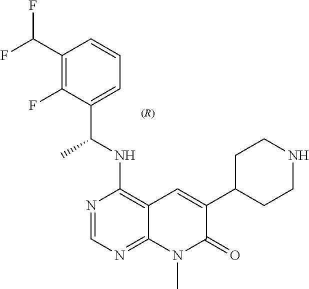 | 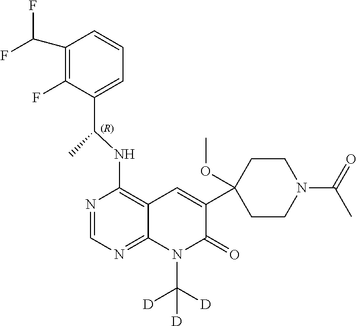 |
| 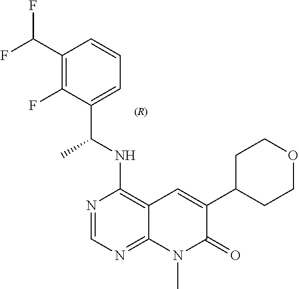 | 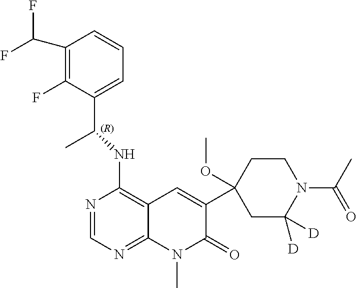 |
| 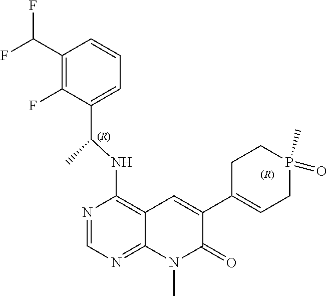 | 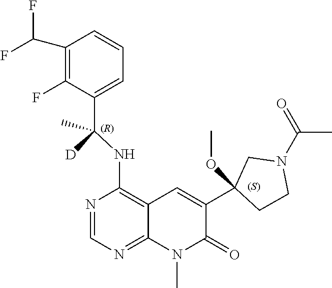 |
| 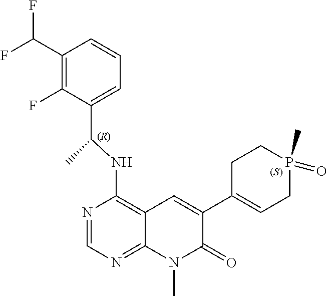 | 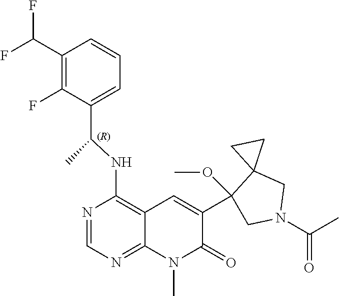 |
| 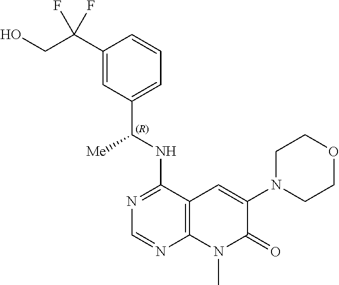 | 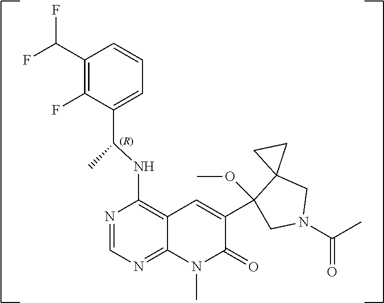 |
| 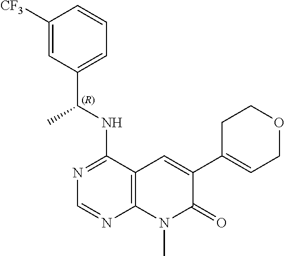 | 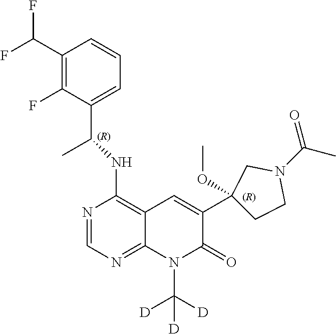 |
| 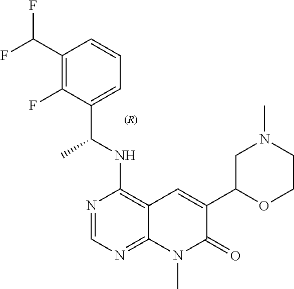 | 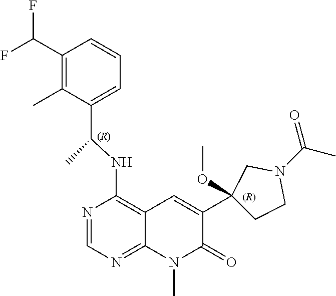 |
| 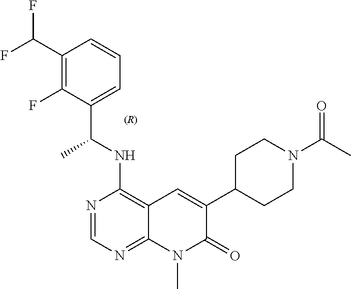 | 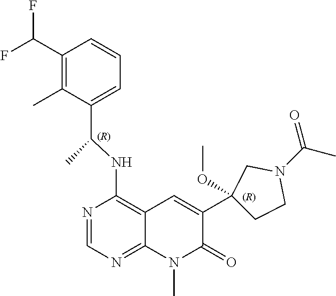 |
| 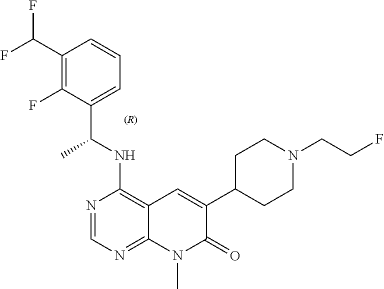 | 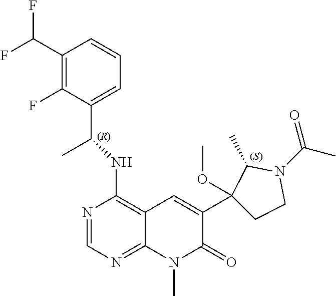 |
| 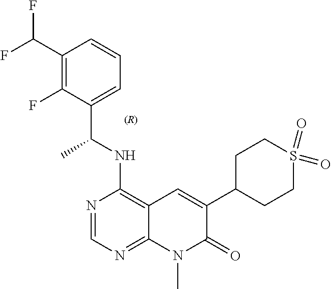 | 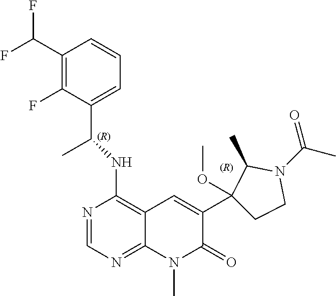 |
| 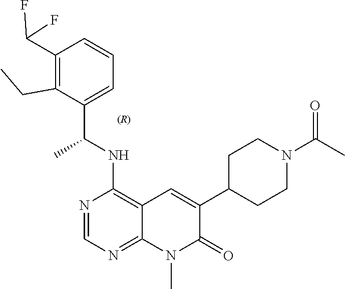 | 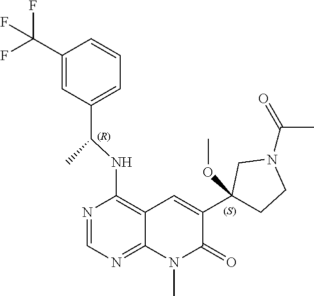 |
| 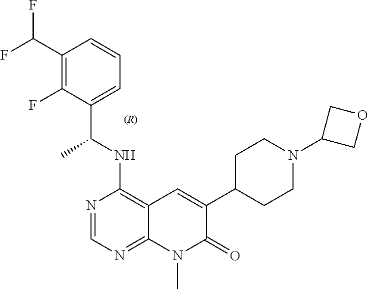 | 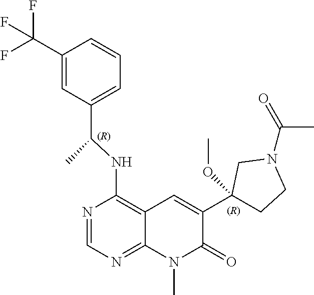 |
| 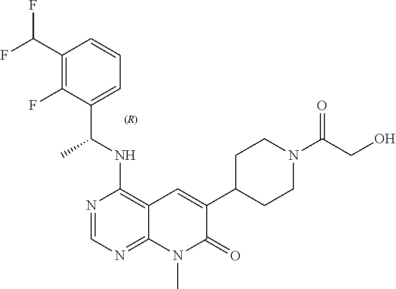 | 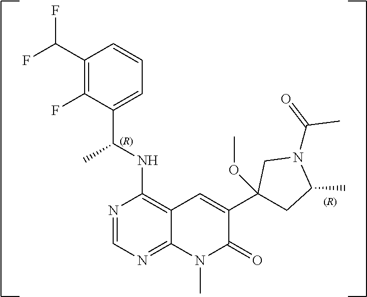 |
| 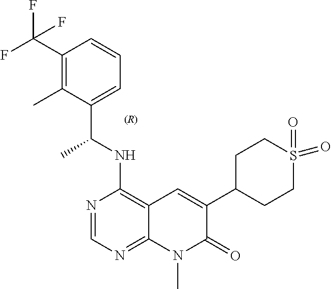 | 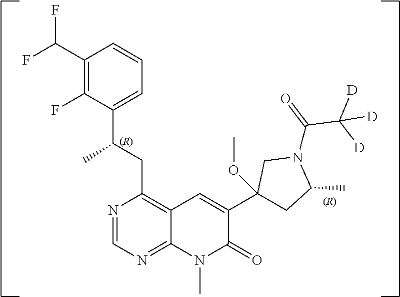 |
| 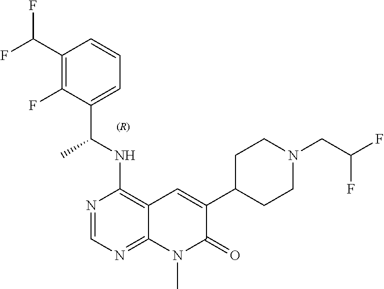 | 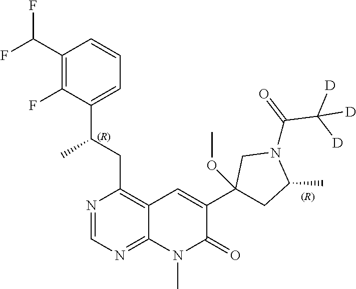 |
| 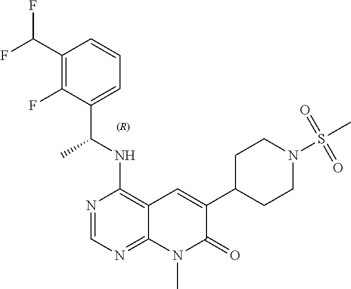 | 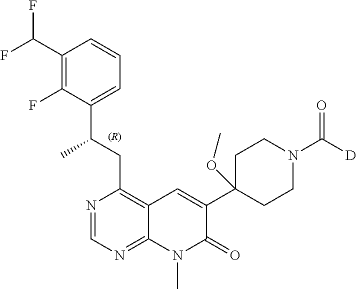 |
| 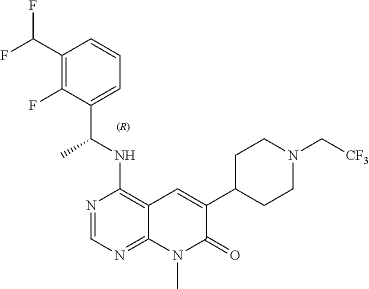 | 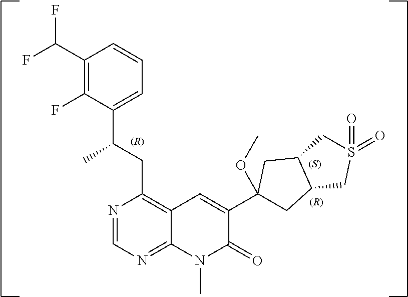 |
| 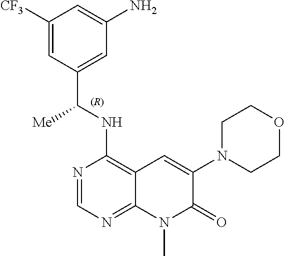 | 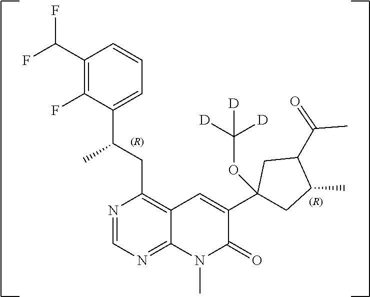 |
| 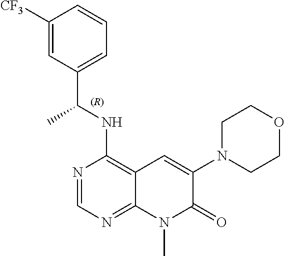 | 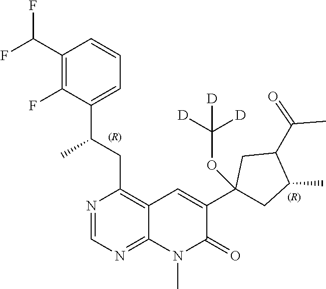 |
| 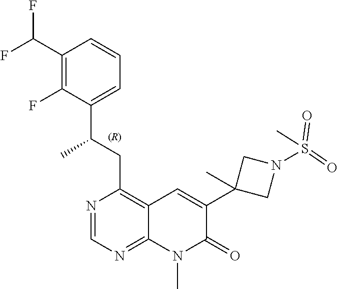 | 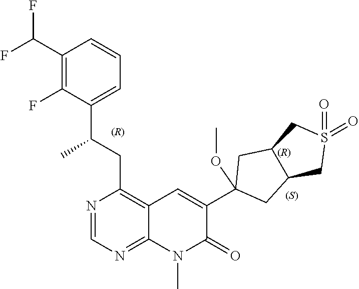 |
| 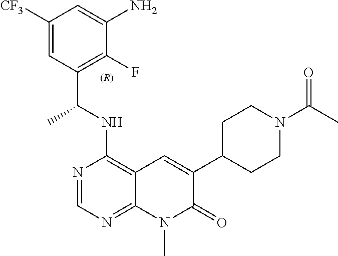 | 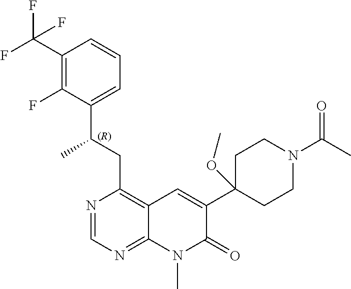 |
| 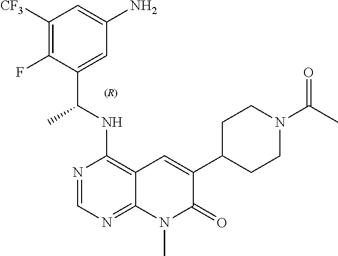 | 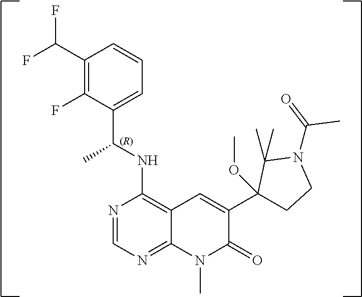 |
| 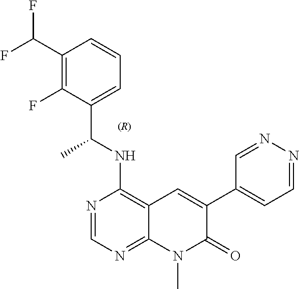 | 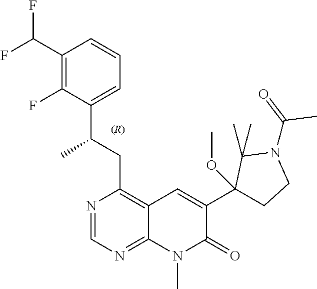 |
| 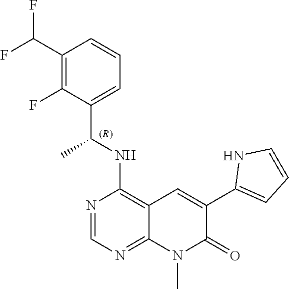 | 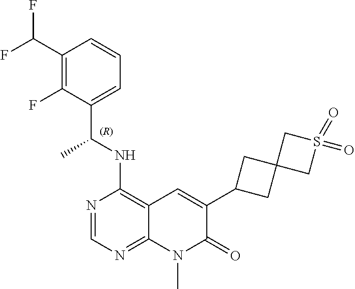 |
| 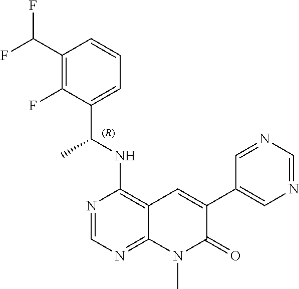 | 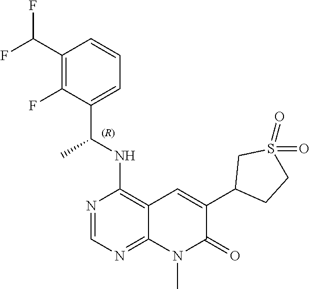 |
| 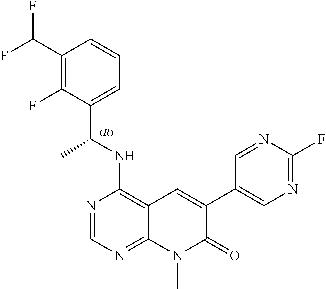 | 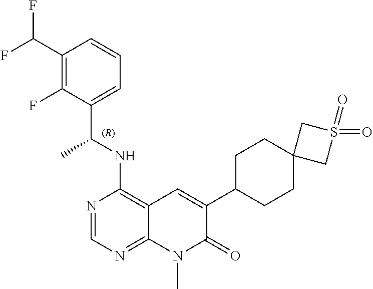 |
| 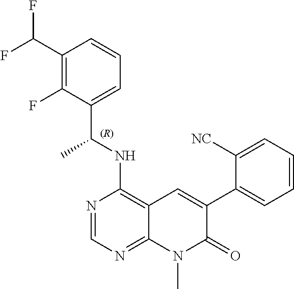 | 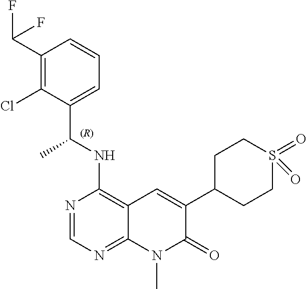 |
| 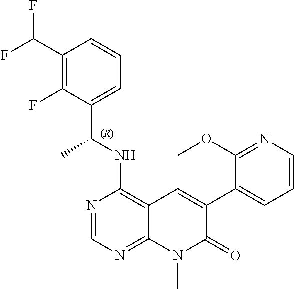 | 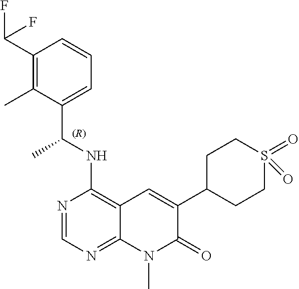 |
| 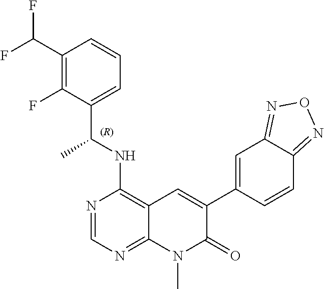 | 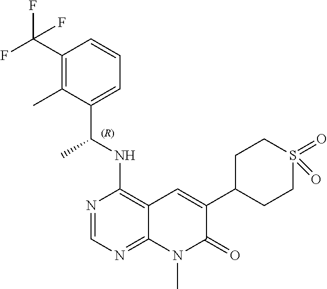 |
| 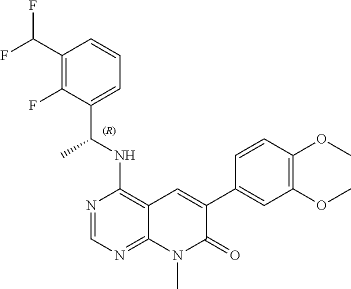 | 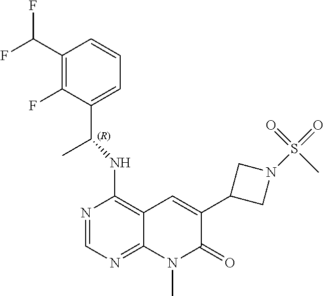 |
| 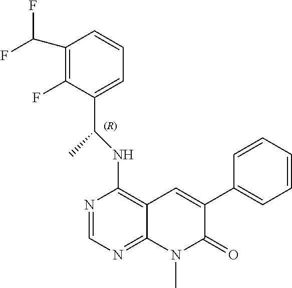 | 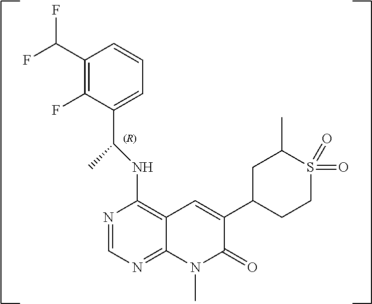 |
| 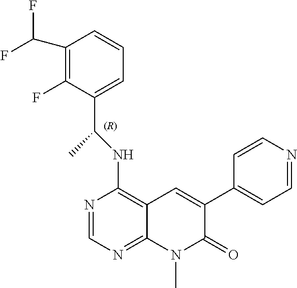 | 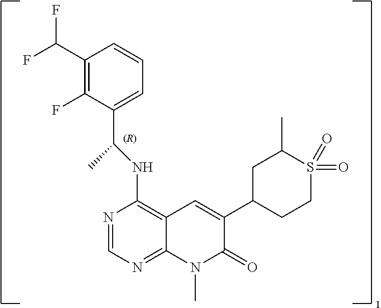 |
| 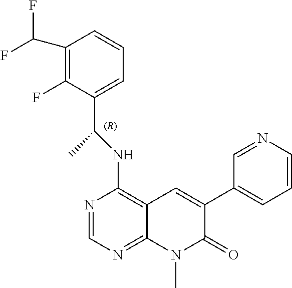 | 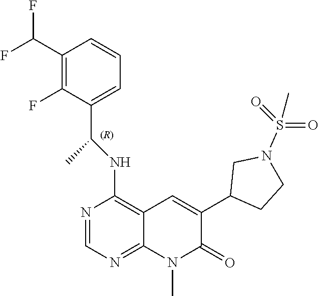 |
| 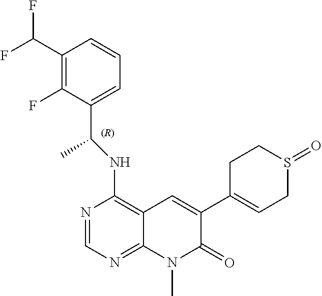 | 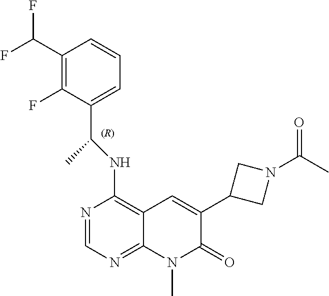 |
| 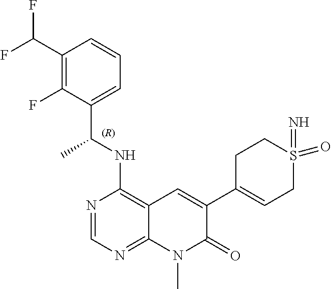 | 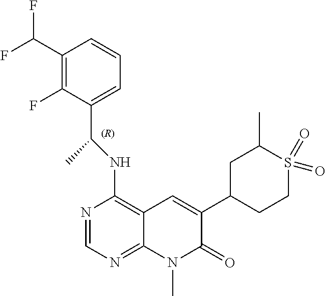 |
| 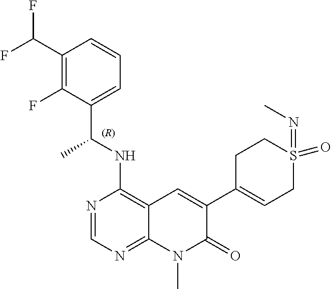 | 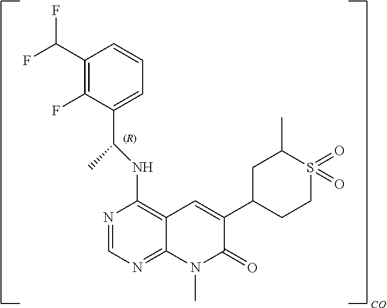 |
| 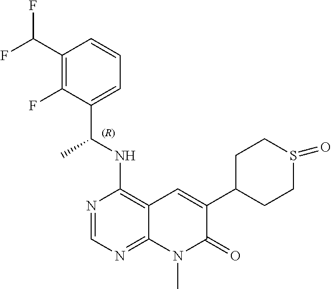 | 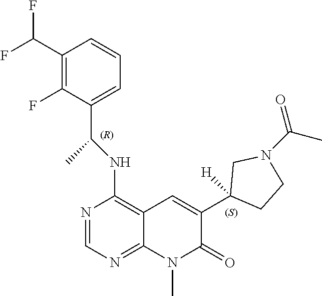 |
| 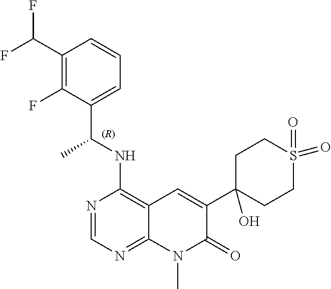 | 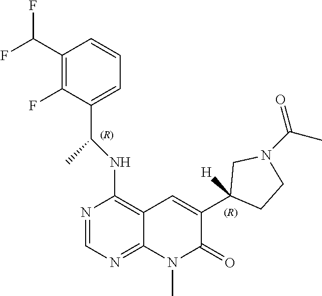 |
| 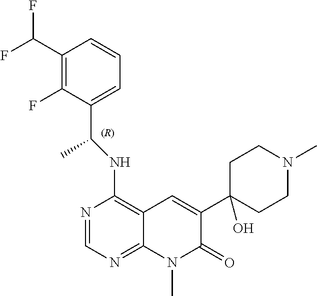 | 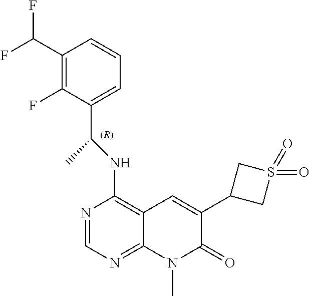 |
| 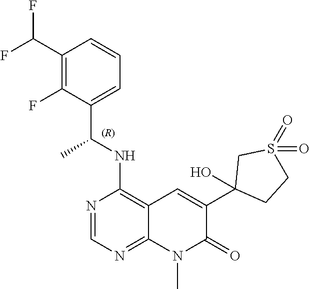 | 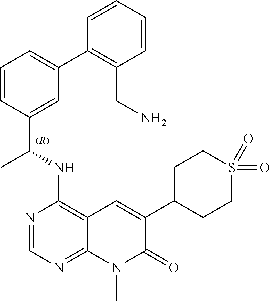 |
| 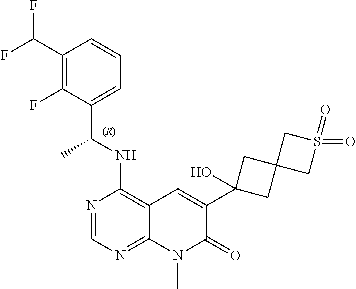 | 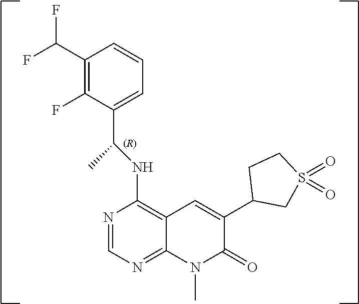 |
| 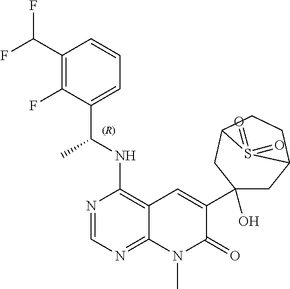 | 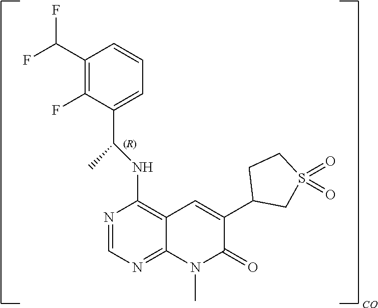 |
| 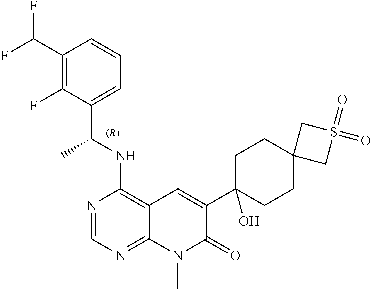 | 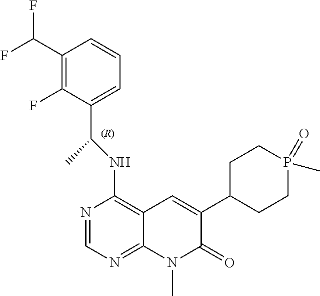 |
| 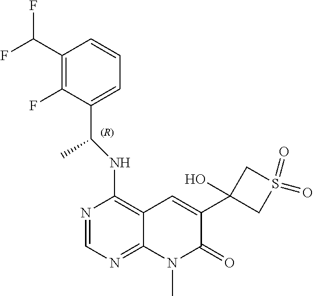 | 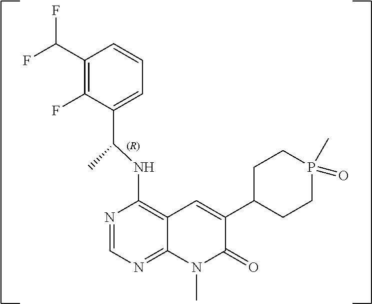 |
| 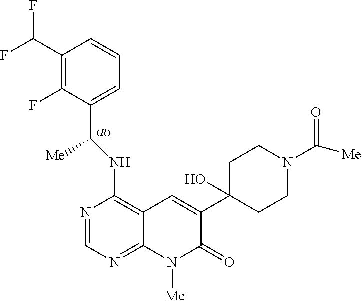 | 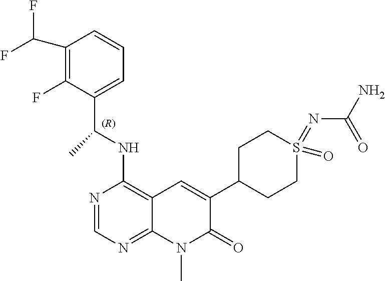 |
| 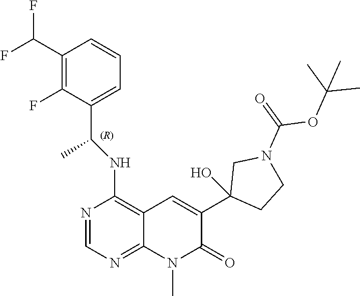 | 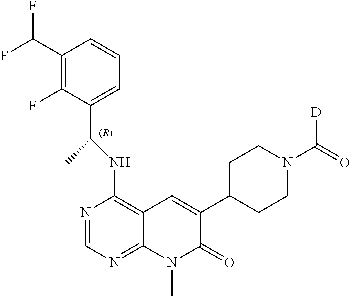 |
| 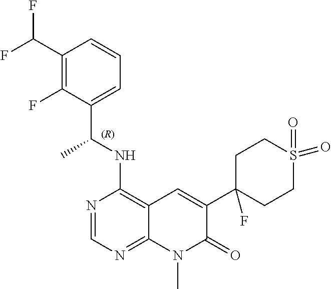 | 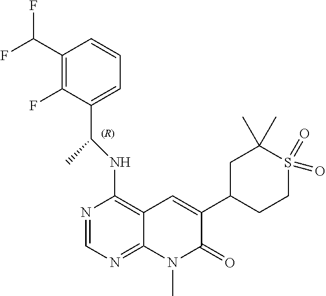 |
| 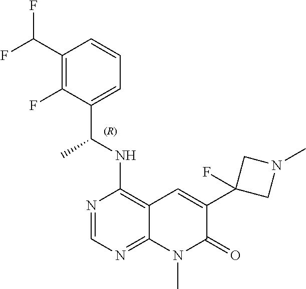 | 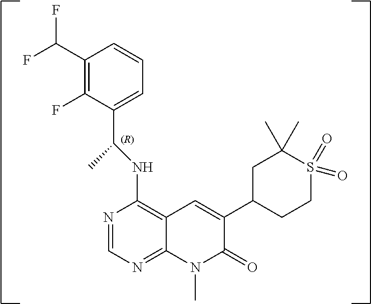 |
| 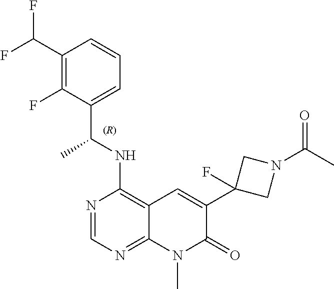 | 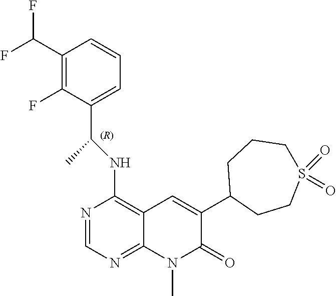 |
| 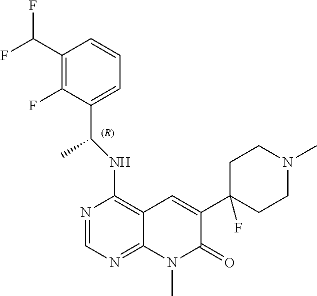 | 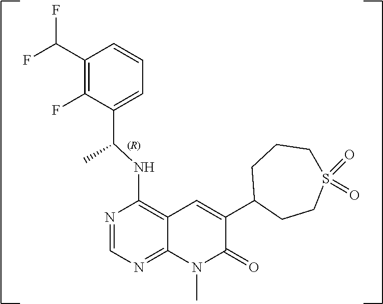 |
| 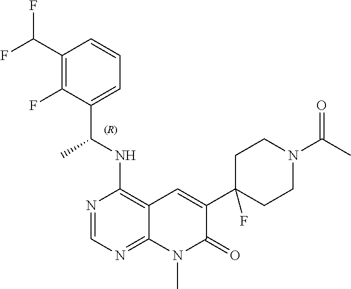 | 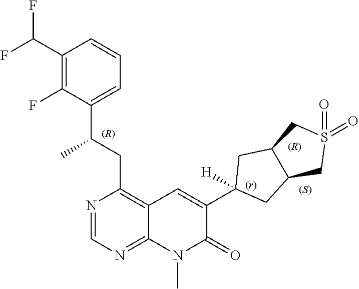 |
| 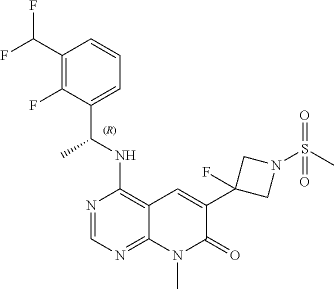 | 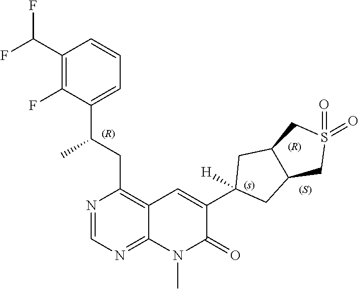 |
| 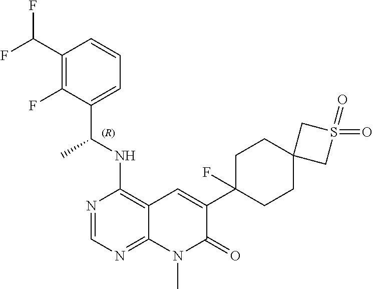 | 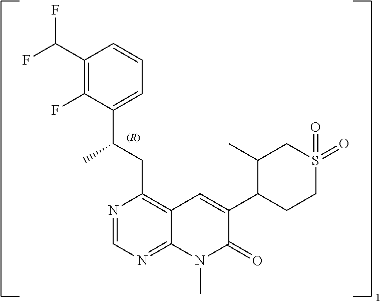 |
| 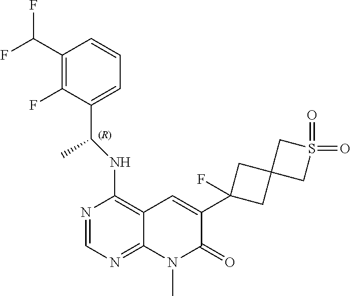 | 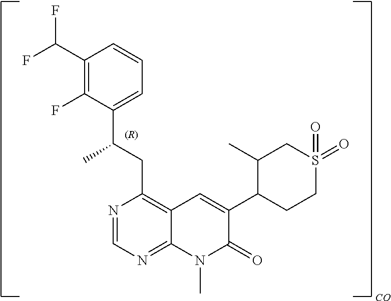 |
| 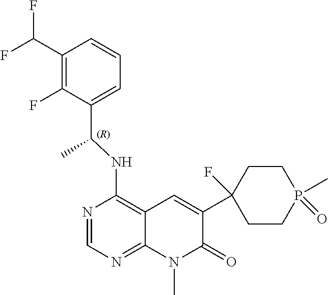 | 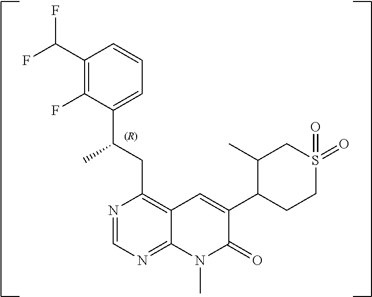 |
| 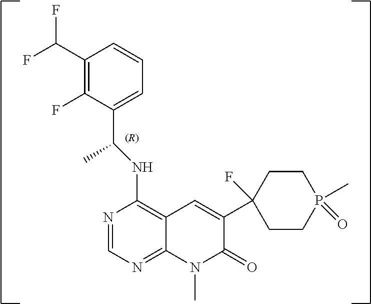 | 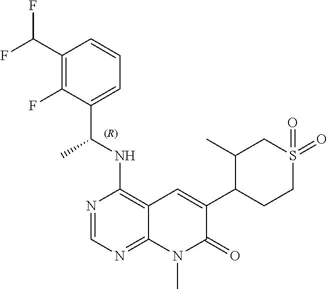 |
| 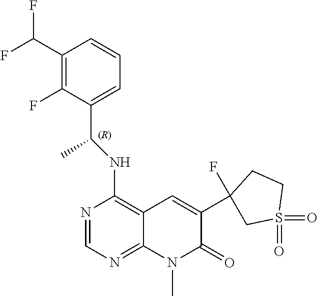 | 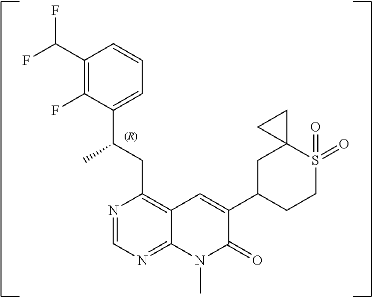 |
| 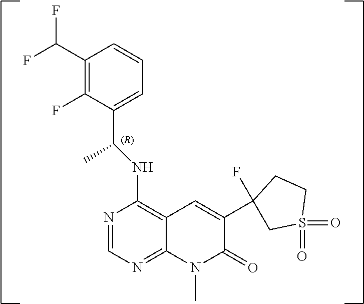 | 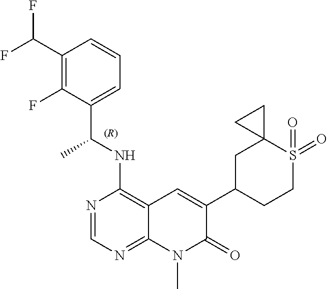 |
| 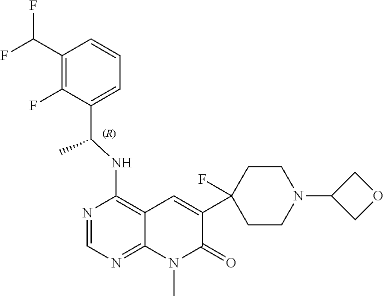 | 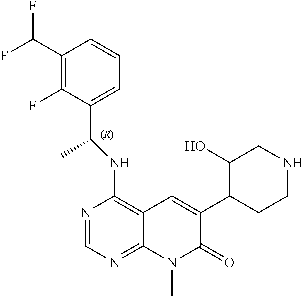 |
| 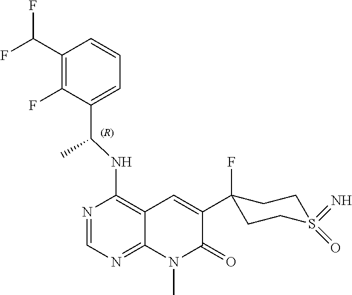 | 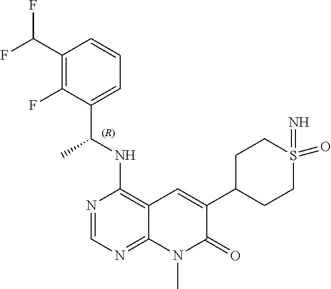 |
| 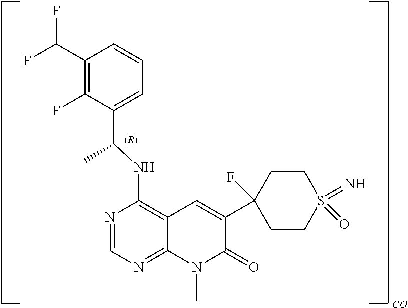 | 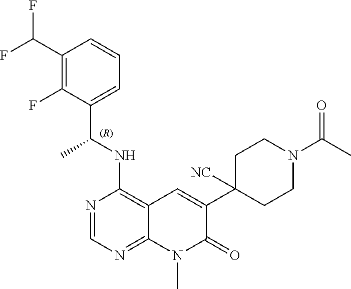 |
| 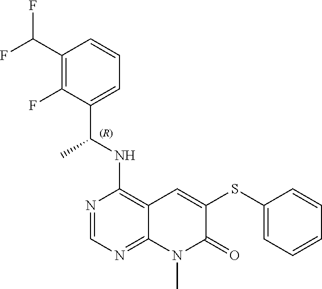 | 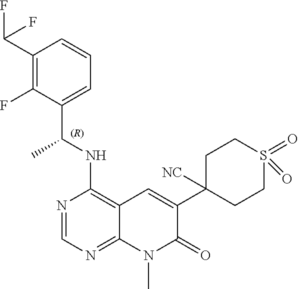 |
| 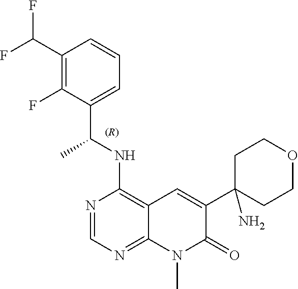 | 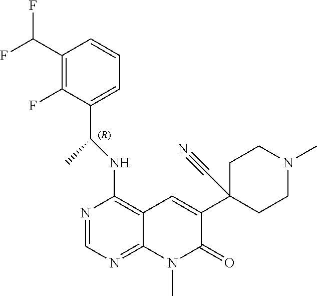 |
| 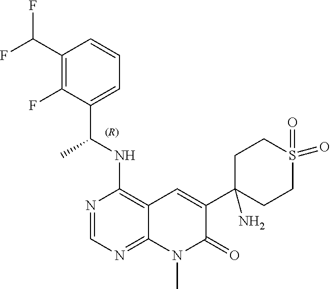 | 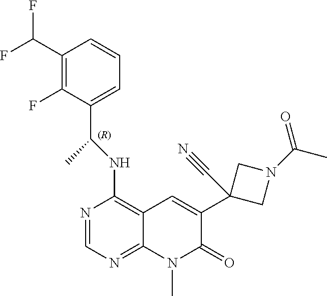 |
| 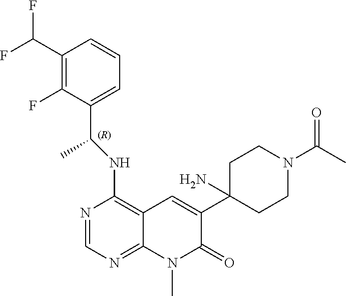 | 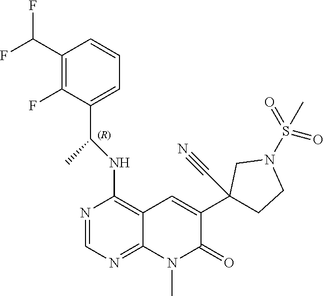 |
| 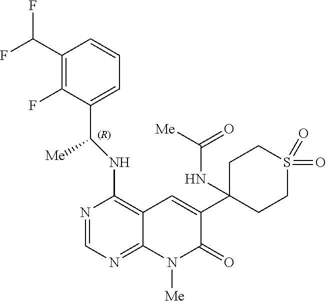 | 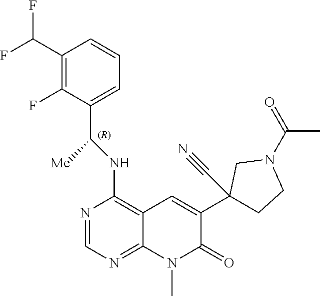 |
| 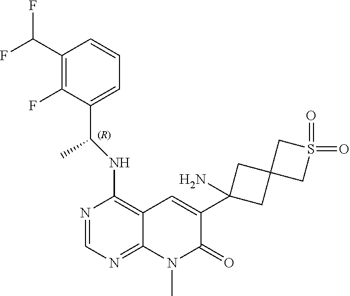 | 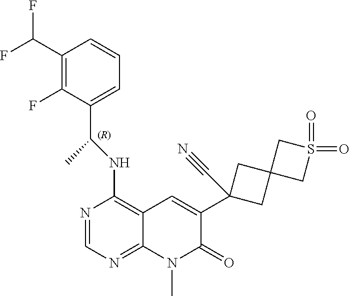 |
| 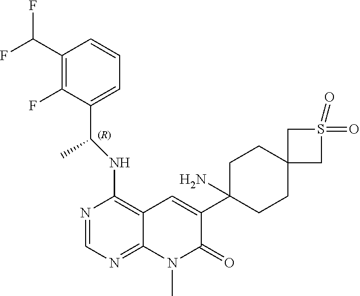 | 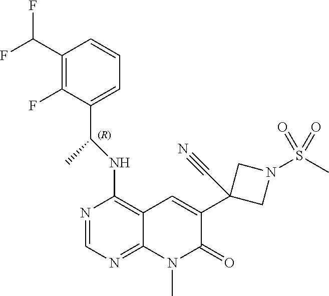 |
| 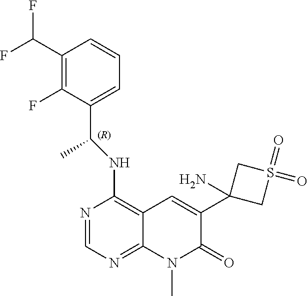 | 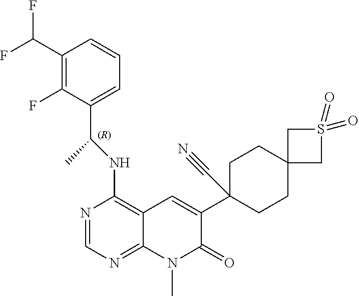 |
| 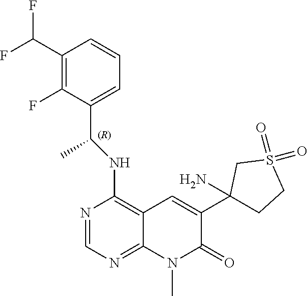 | 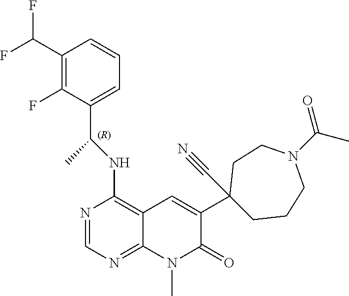 |
| 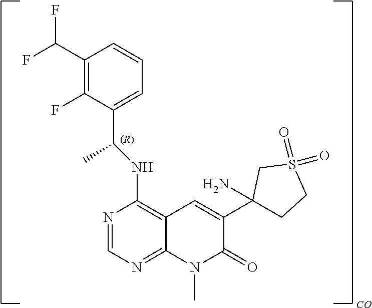 | 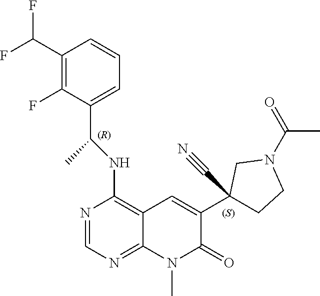 |
| 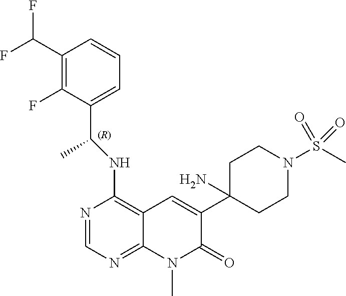 | 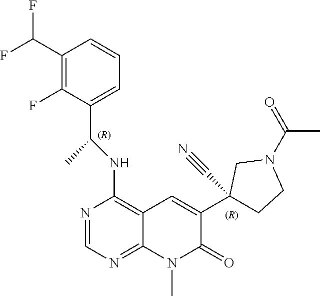 |
| 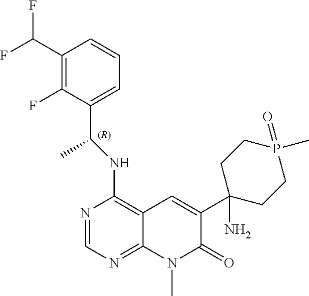 | 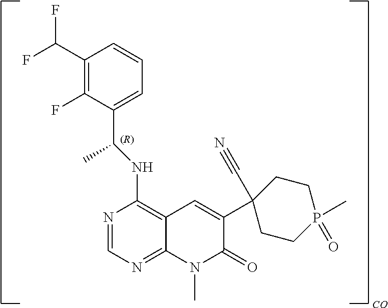 |
| 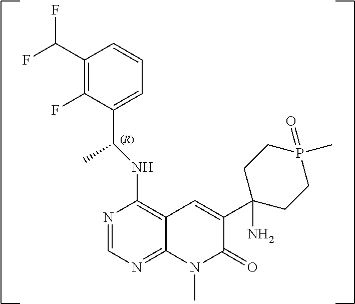 | 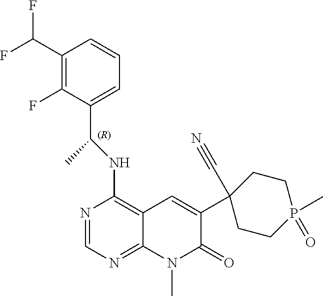 |
| 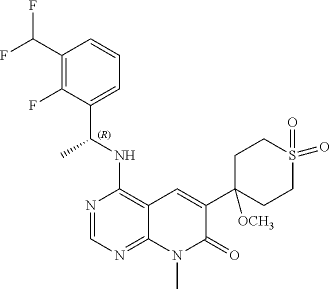 | 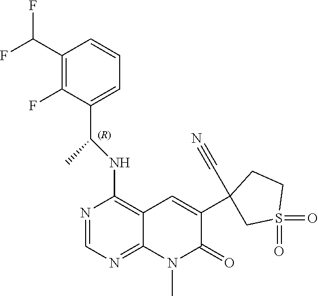 |
| 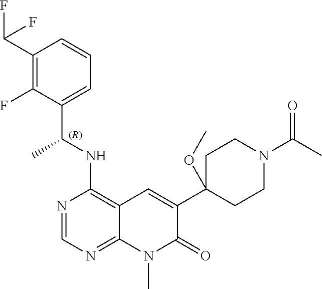 | 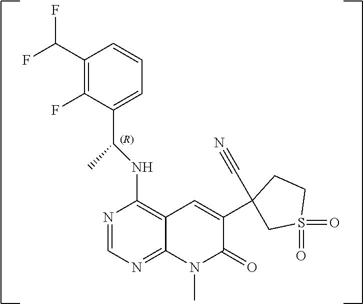 |
| 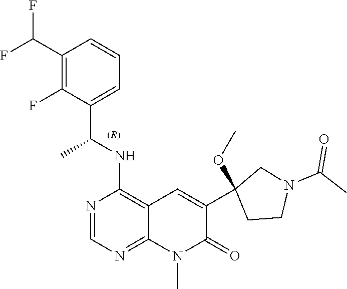 | 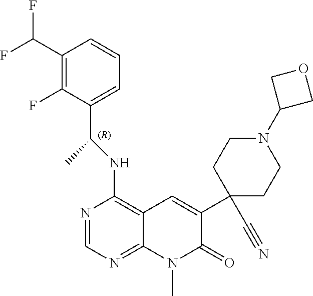 |
| 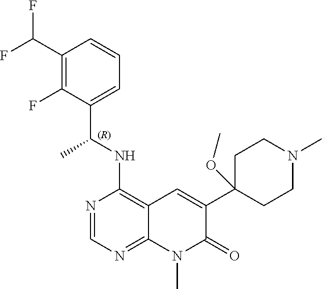 | 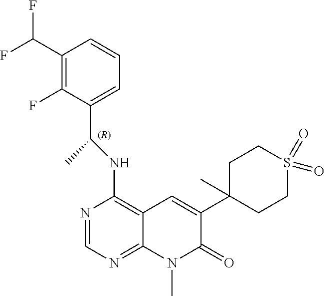 |
| 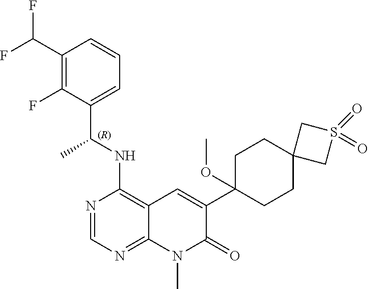 | 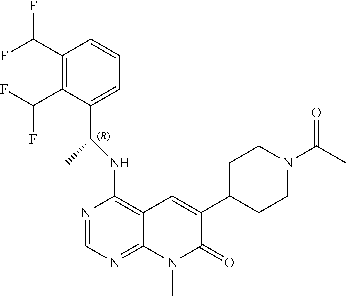 |
| 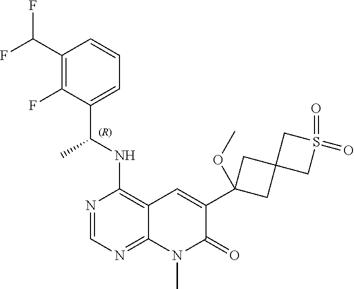 | 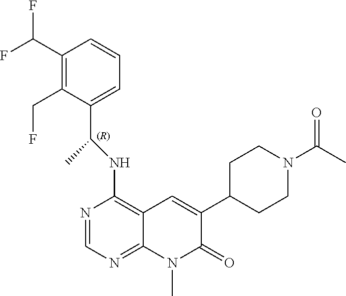 |
| 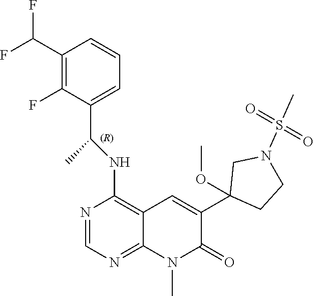 | 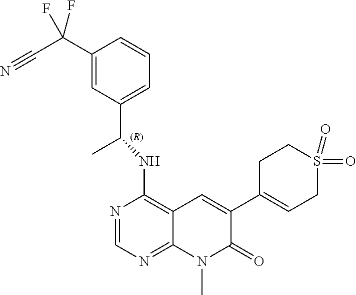 |
| 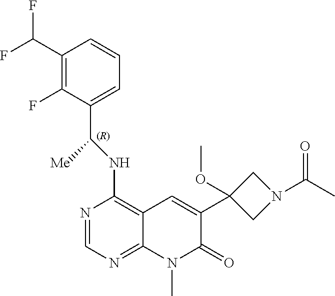 | 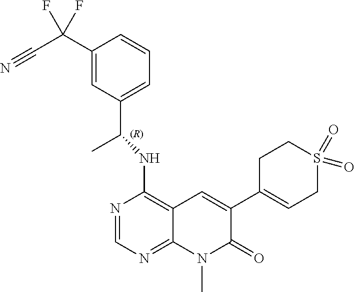 |
| 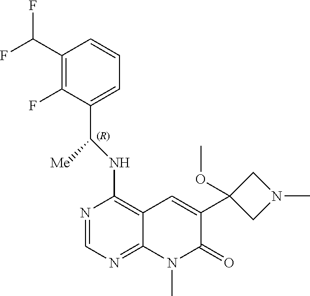 | 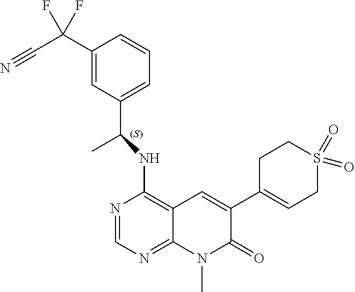 |
| 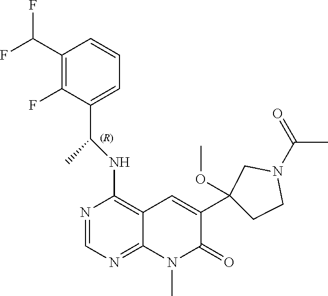 | 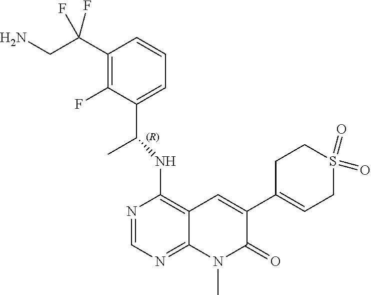 |
| 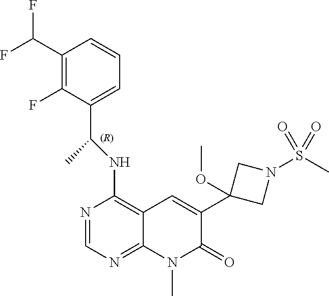 | 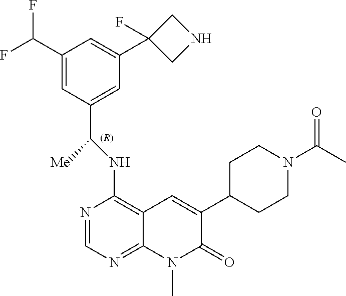 |
| 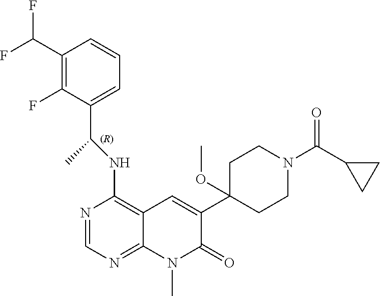 | 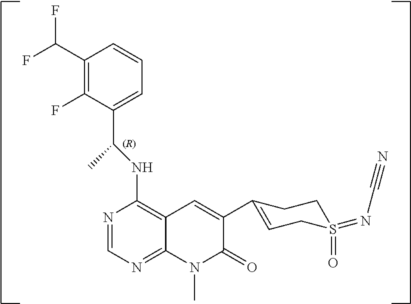 |
| 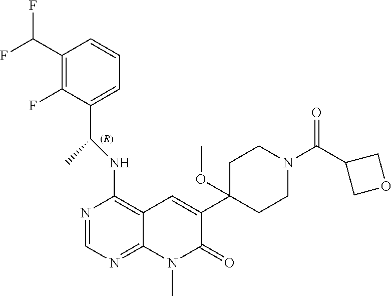 | 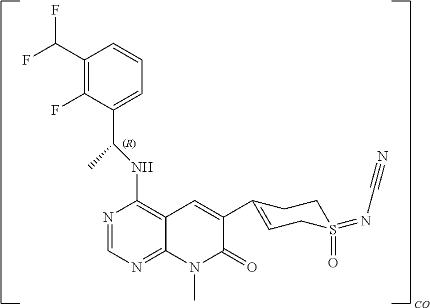 |
| 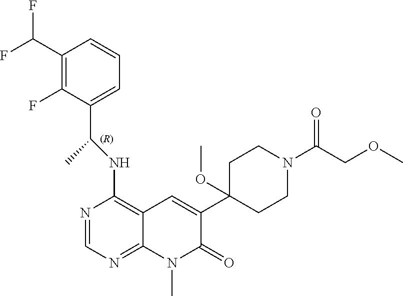 | 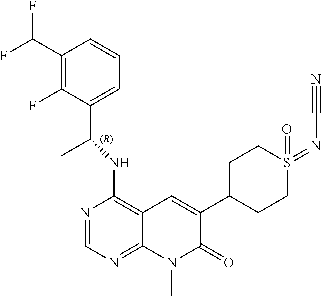 |
| 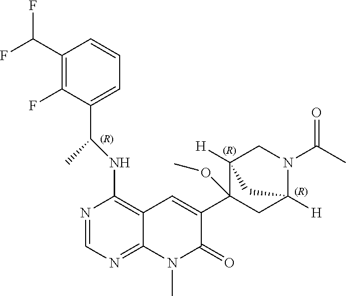 | 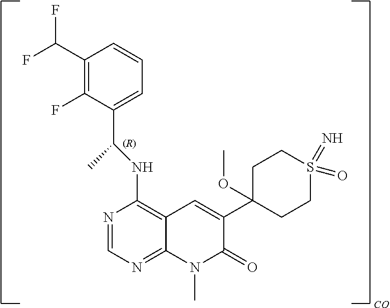 |
| 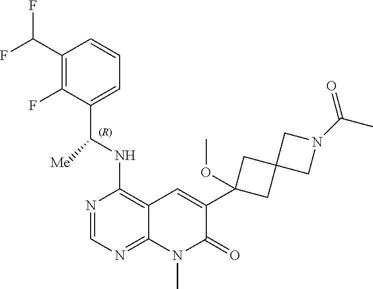 | 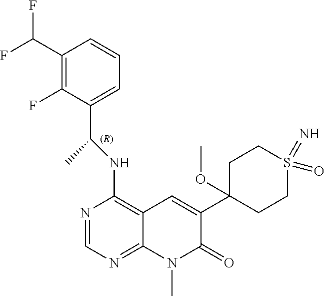 |
| 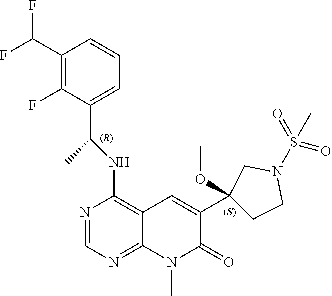 | 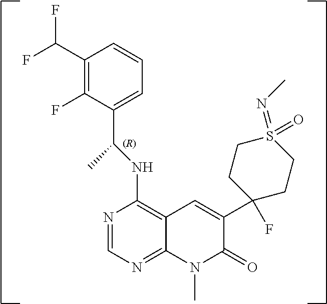 |
| 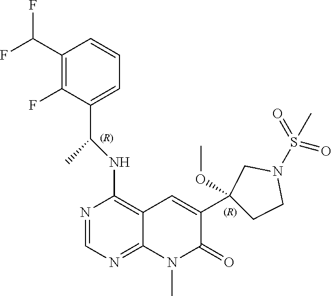 | 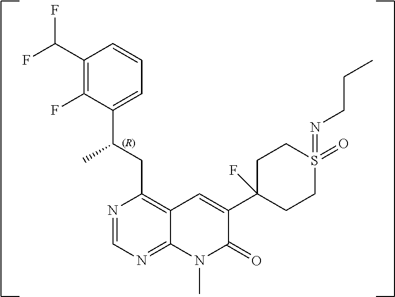 |
| 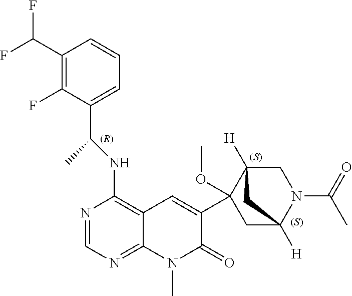 | 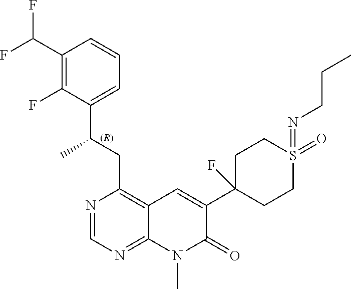 |
| 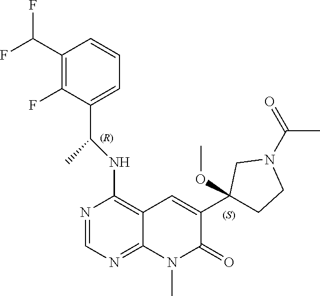 | 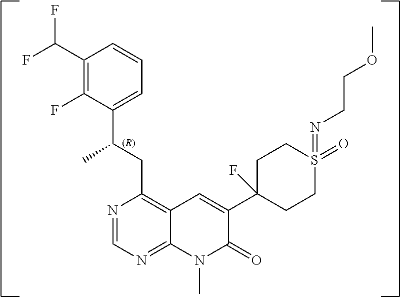 |
| 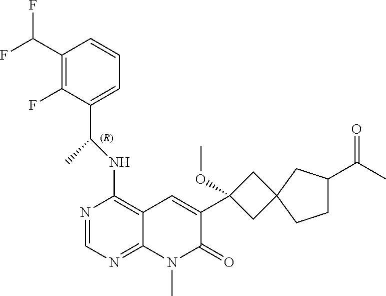 | 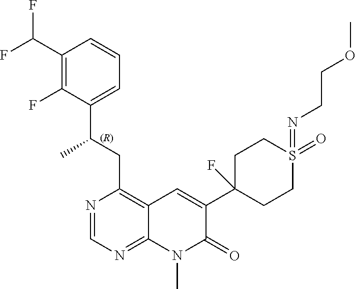 |
| 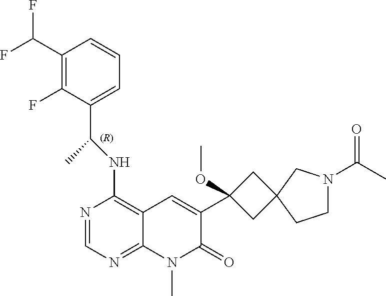 | 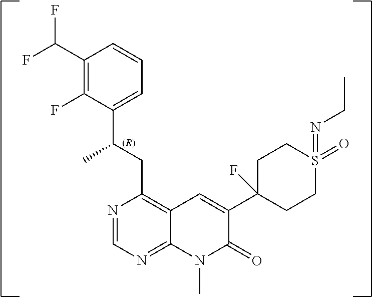 |
| 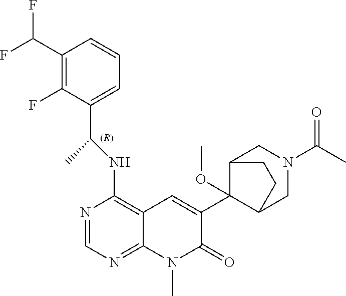 | 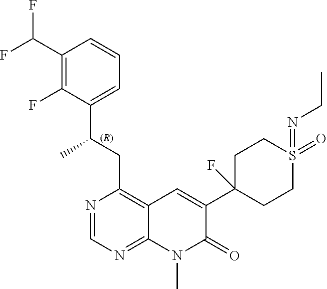 |
| 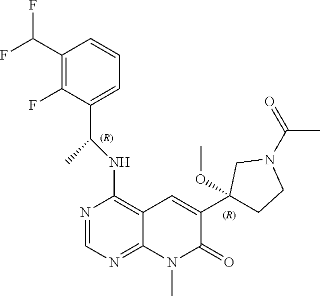 | 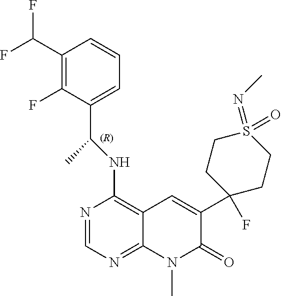 |
| 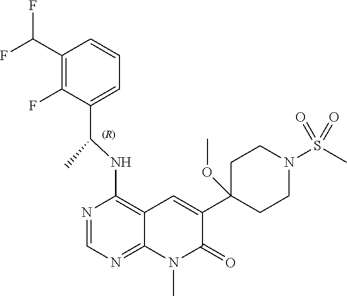 | 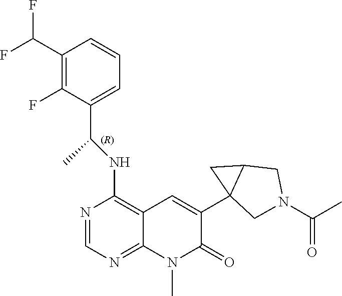 |
| 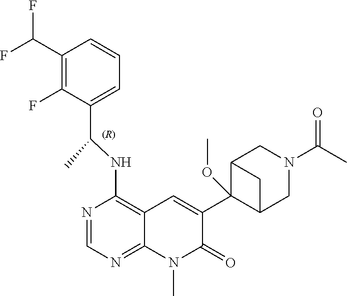 | 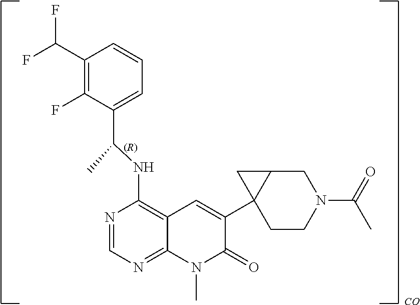 |
| 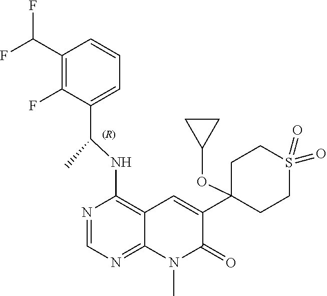 | 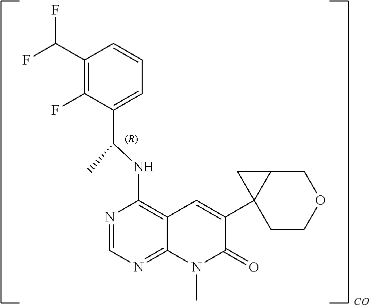 |
| 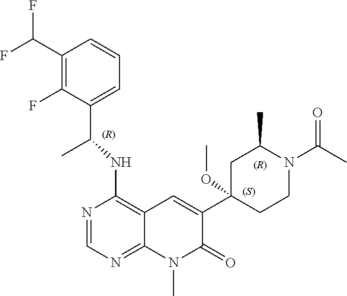 | 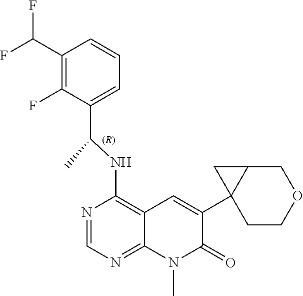 |
| 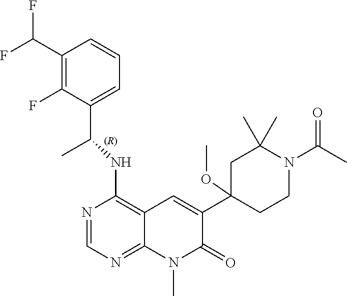 | 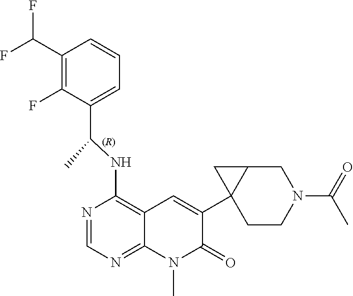 |
| 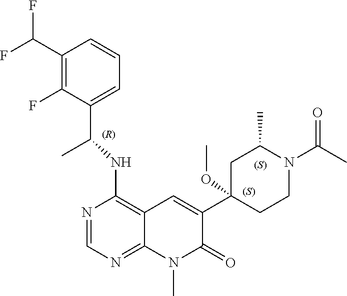 | 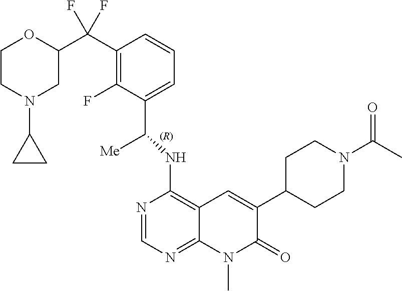 |
| 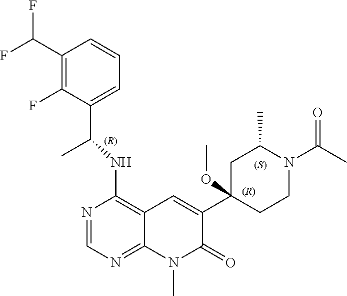 | 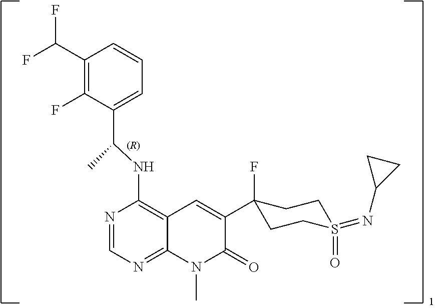 |
| 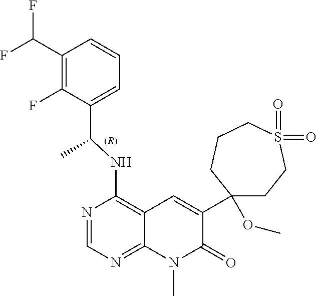 | 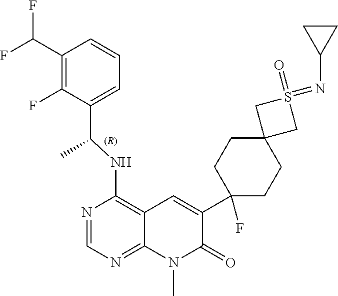 |
| 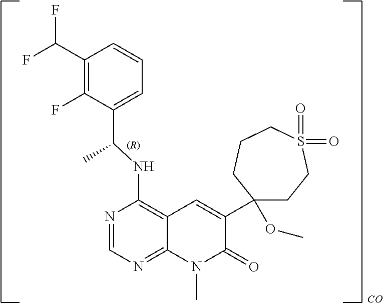 | 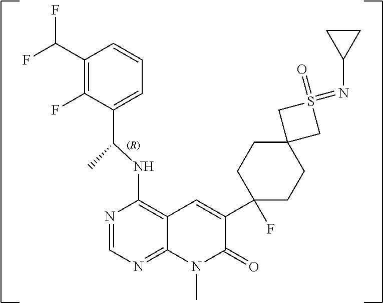 |
| 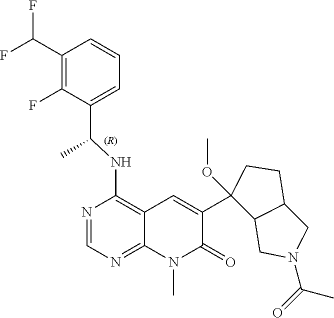 | 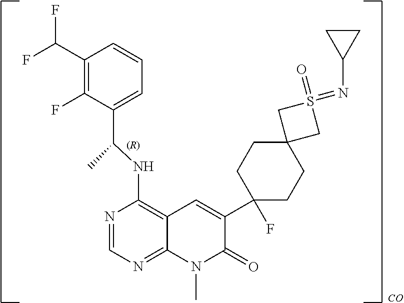 |
| 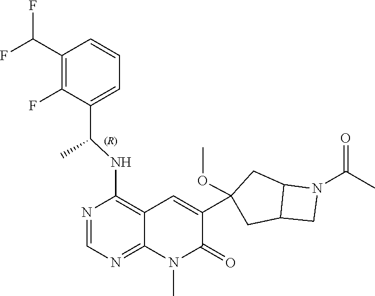 | 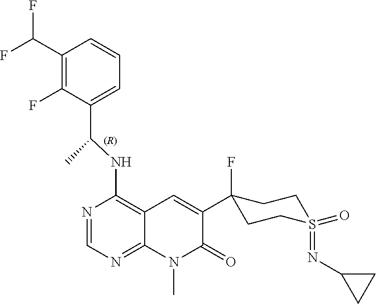 |
| 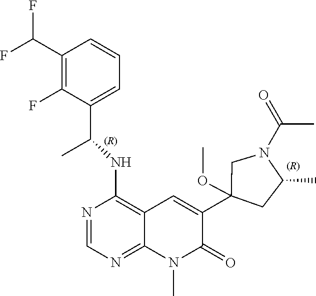 | 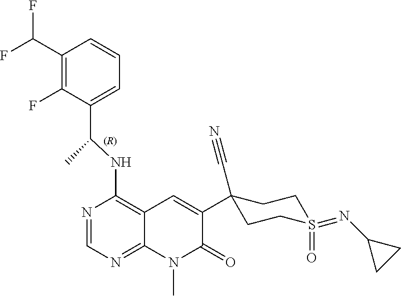 |
| 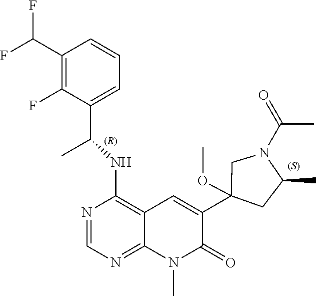 | 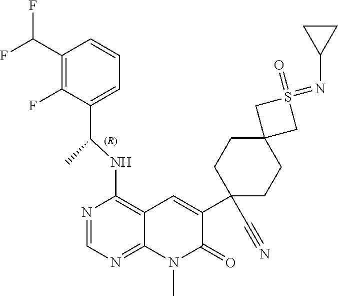 |
| 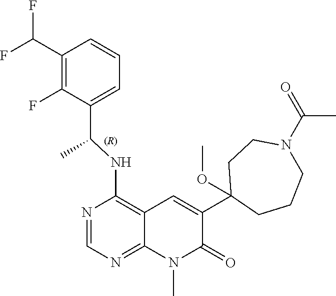 | 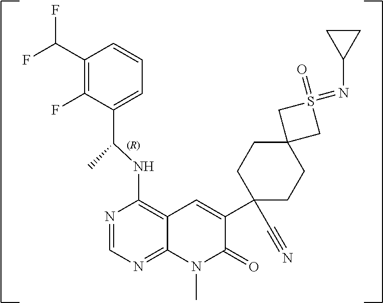 |
| 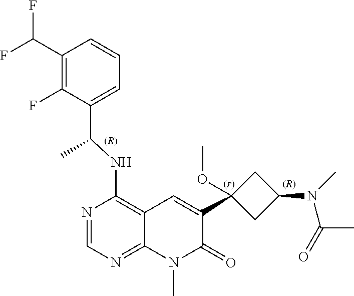 | 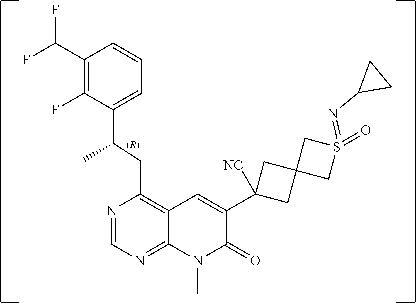 |
| 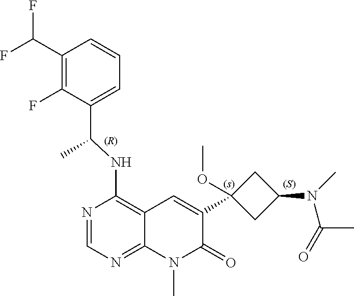 | 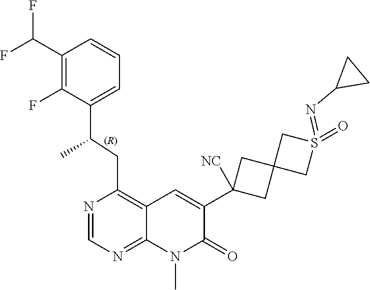 |
| 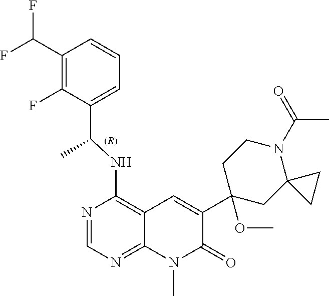 | 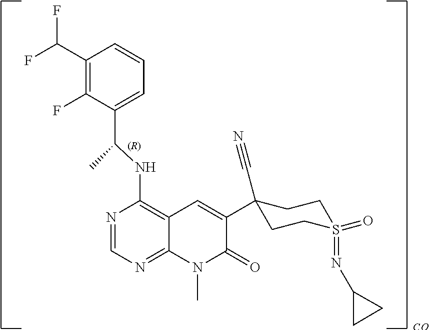 |
| 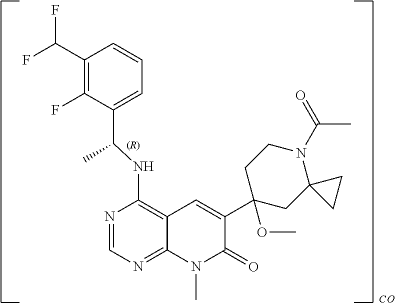 | 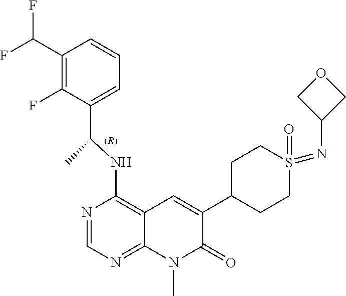 |
| 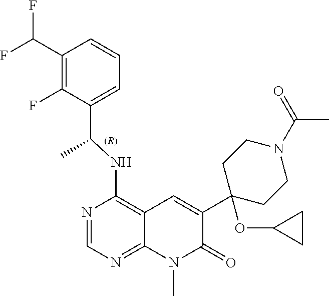 | 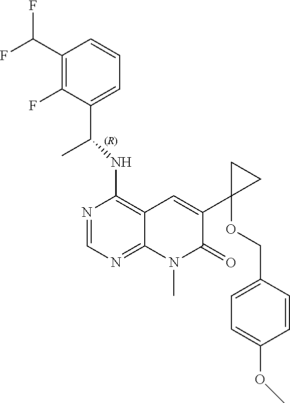 |
| 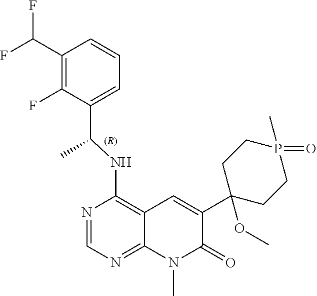 | 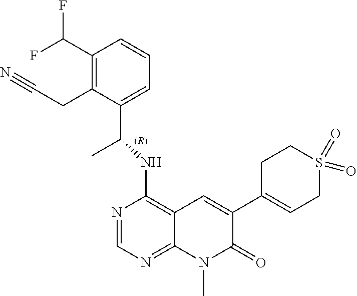 |
| 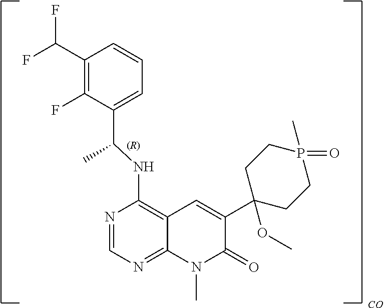 | 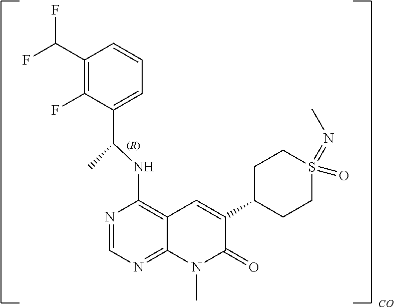 |
| 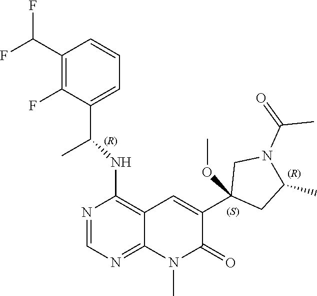 | 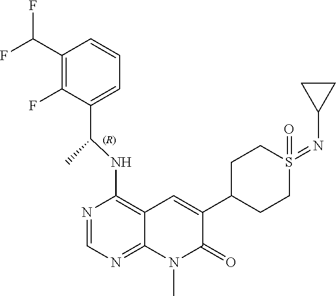 |
| 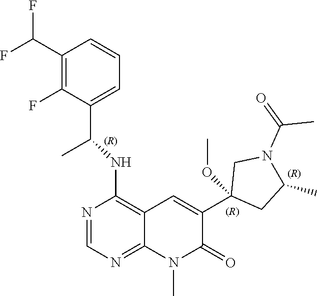 | 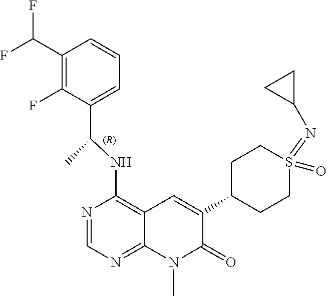 |
| 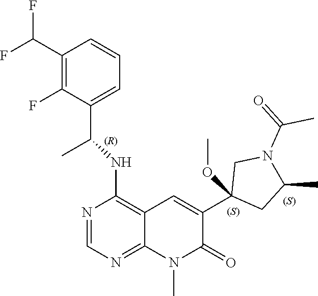 | 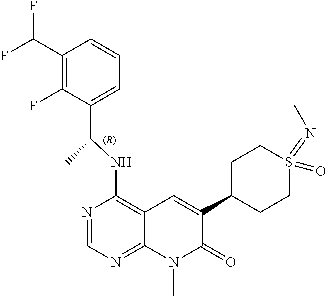 |
| 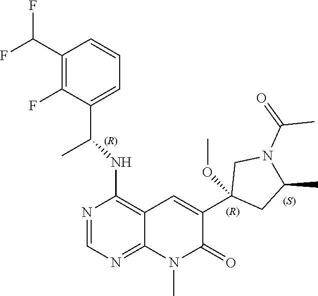 | 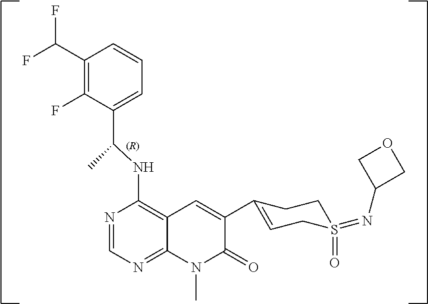 |
| 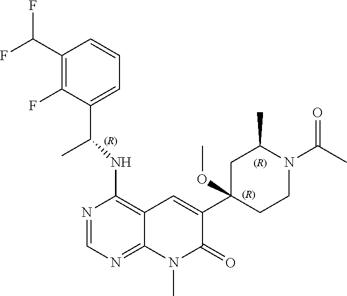 | 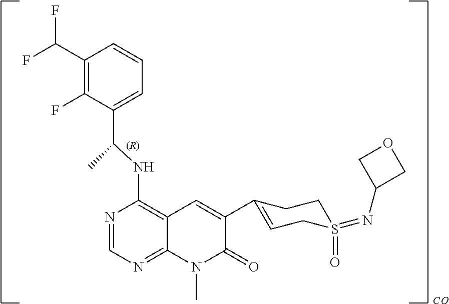 |
| 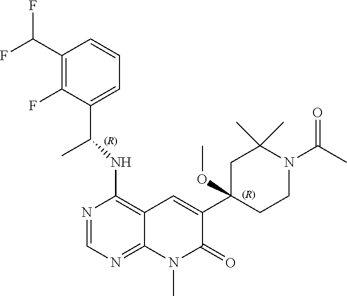 | 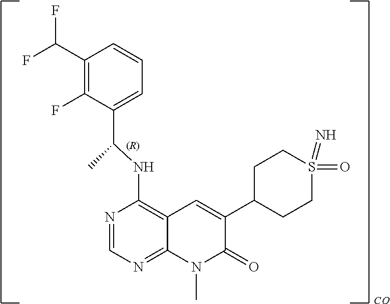 |
| 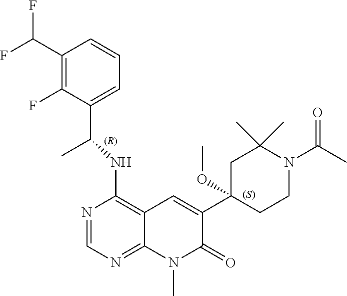 | 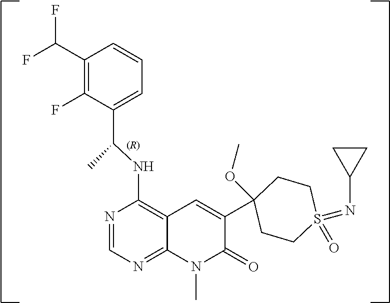 |
| 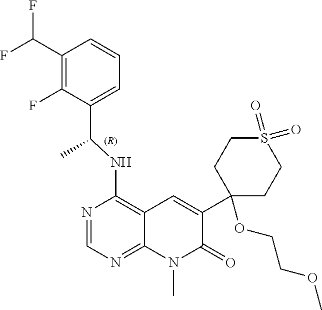 | 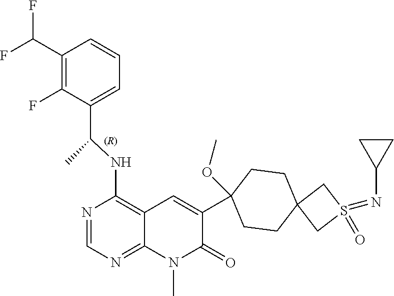 |
| 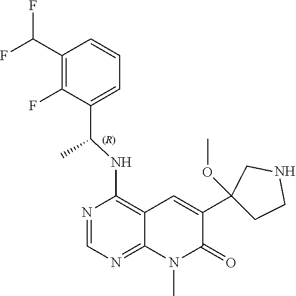 | 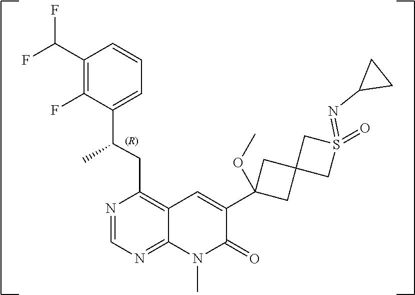 |
| 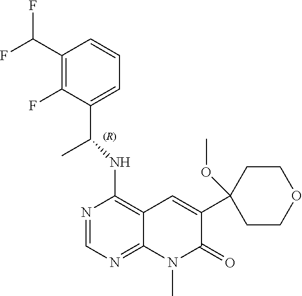 | 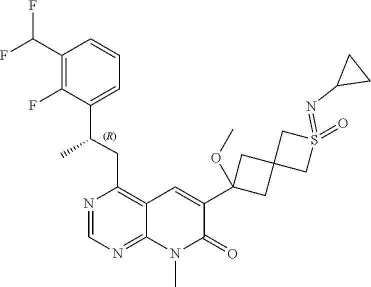 |
| 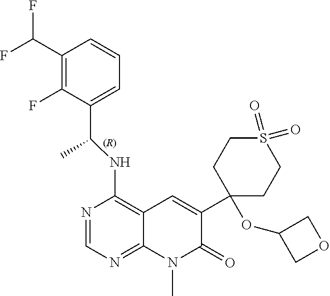 | 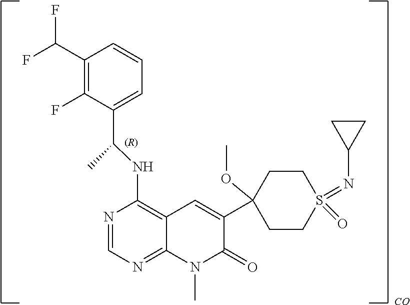 |
| 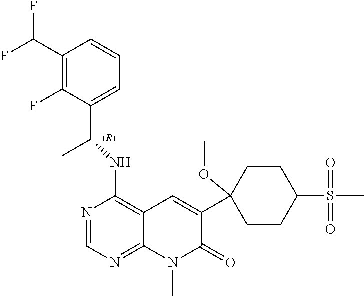 | 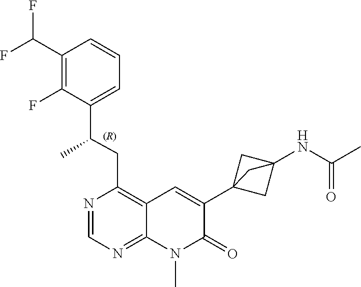 |
| 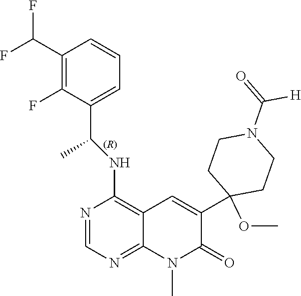 | 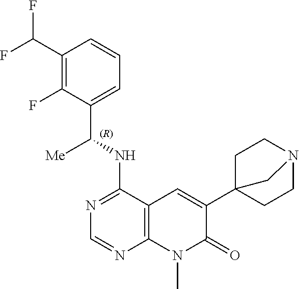 |
| 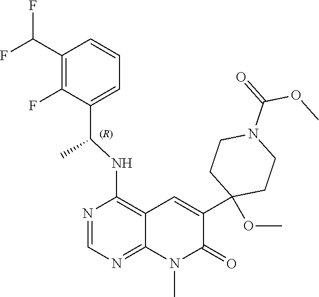 | 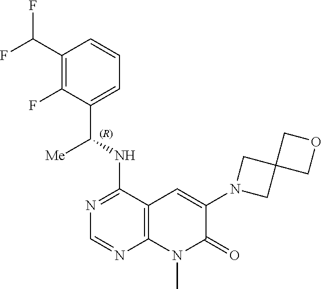 |
| 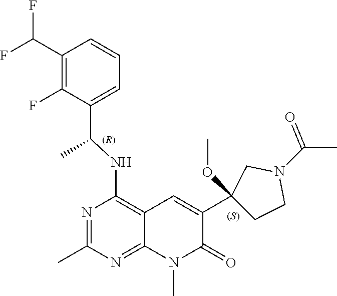 | 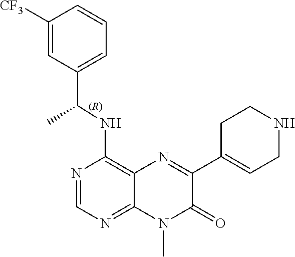 |
| 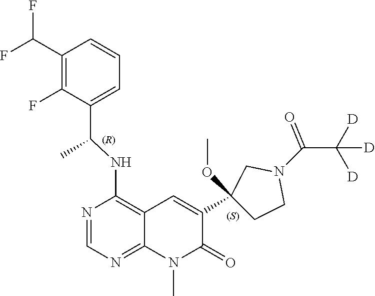 | 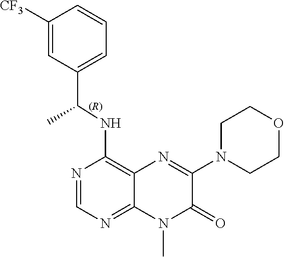 |
| 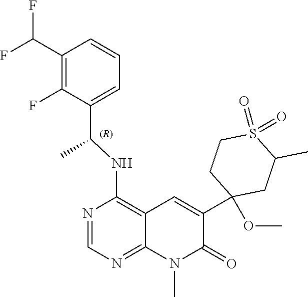 | |

Note: any compound shown in the foregoing table may be a diastereomer, and the absolute stereochemistry of such diastereomer may not be known.

In some embodiments, the SOS1 inhibitor is a compound selected from the group consisting of the compounds in the following table, or a pharmaceutically acceptable salt or stereoisomer thereof:

Structure

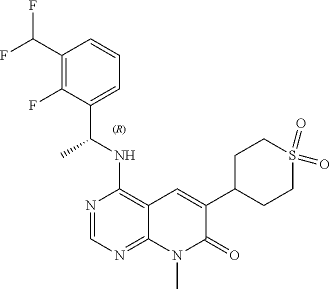

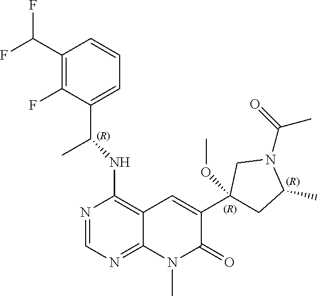

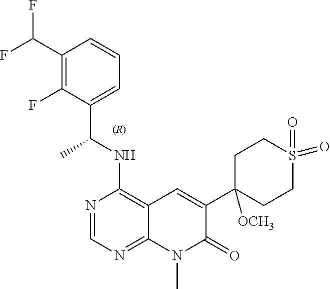

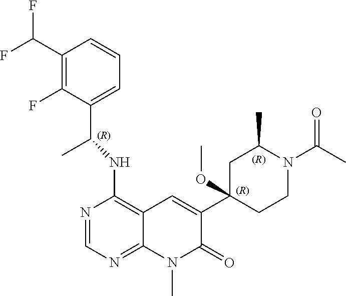

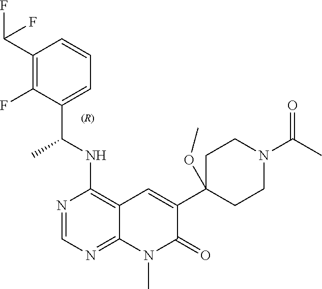

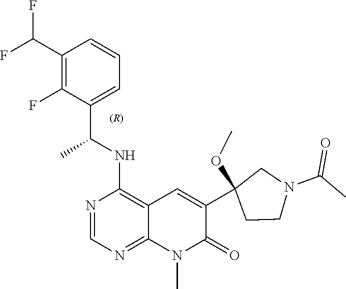

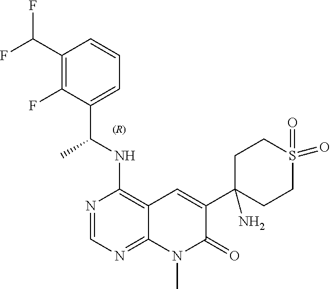

Note:
any compound shown in the foregoing table may be a diastereomer,
and the absolute stereochemistry of such diastereomer may not be
shown.

In some embodiments, the SOS1 inhibitor is a compound having the structure of Formula (53-I), (53-II), or (53-III):

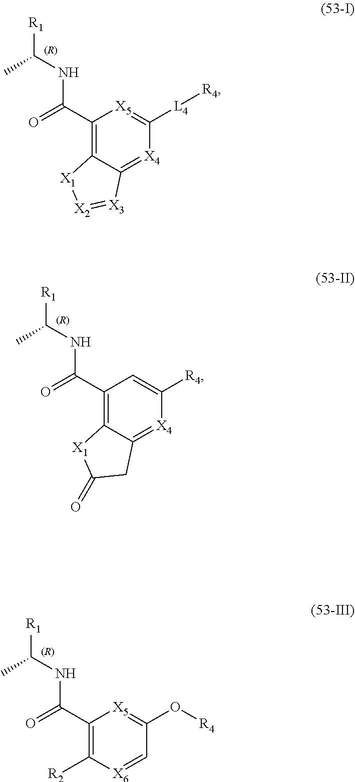

or a pharmaceutically acceptable salt, solvate, isomer (e.g., stereoisomer), prodrug, or tautomer thereof, wherein:
X¹ is NH or S;
X² is CH or N;
X³ is CH or N;
X⁴ is CR₃ or N;
X⁵ is CH or N;
X⁶ is CH or N;
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, —NH—C₁₋₆ alkyl, and —NH₂;
R₃ is selected from the group consisting of H, —O—C₁₋₆ alkyl, and —O—C₁₋₆ heteroalkyl;
L₄ is a bond or O; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with one or more C₁₋₆ alkyl, —R₄ₐ, —OR₄ₐ, —O—C₁₋₆ alkyl-R₄ₐ, ═O, halogen, —C(O)R₄ₐ, —C(O)OR₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R⁴a, 3-7 membered heterocyclyl optionally substituted with R₄ₐ, 6-10 membered aryl optionally substituted with R₄ₐ, or 5-10 membered heteroaryl optionally substituted with R₄ₐ;
wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂) OCH₃, or —(CH₂) OH, wherein r is 1, 2, or 3;
wherein each R_{4b} is independently H, C₁₋₆ alkyl; and
   wherein each R_{4c} is independently H or C₁₋₆ alkyl.

In some embodiments, the SOS1 inhibitor is a compound having the structure of Formula (53-Ia), (53-IIa), or (53-IIIa):

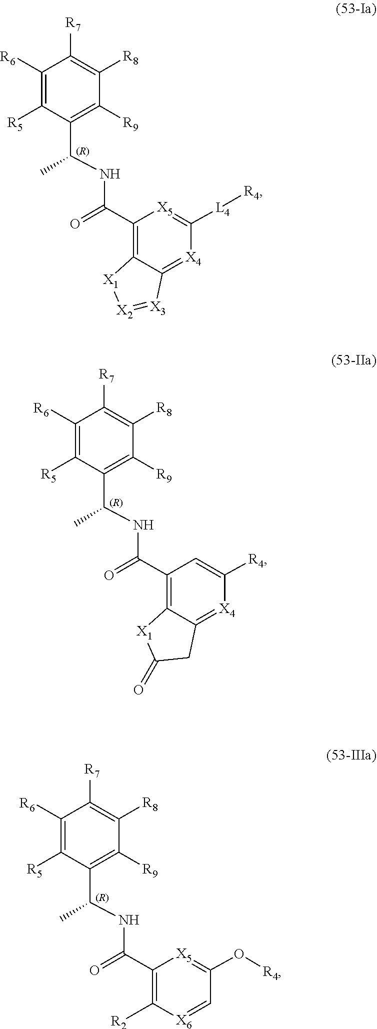

or a pharmaceutically acceptable salt, solvate, isomer (e.g., stereoisomer), prodrug, or tautomer thereof, wherein X¹, X², X³, X⁴, X⁵, X⁶, R₂, L₄, and R₄ are as defined in Formula (53-I), (53-II), or (53-III);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with-OH, C₁₋₆ alkyl optionally substituted with —R₁₀, halogen, —NO₂, ═O, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S (O) NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R₅, R₆, R₇, R₈, and R₉ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with-OH, —SH, —NH₂, —NO₂, or —CN.

In some embodiments, the SOS1 inhibitor is a compound having the structure of Formula (53-II-1):

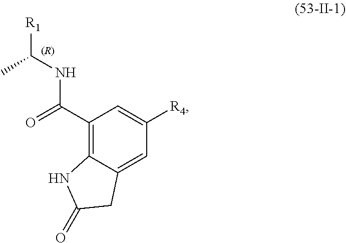

or a pharmaceutically acceptable salt, solvate, isomer (e.g., stereoisomer), prodrug, or tautomer thereof, wherein R₁ and R₄ are as defined in Formula (II).

In some embodiments, the SOS1 inhibitor is a compound selected from the group consisting of the compounds in the following table, or a pharmaceutically acceptable salt, solvate, isomer (e.g., stereoisomer), prodrug, or tautomer thereof:

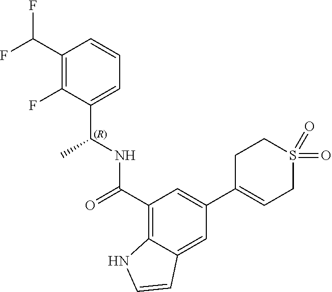

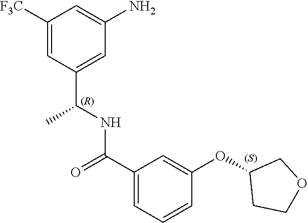

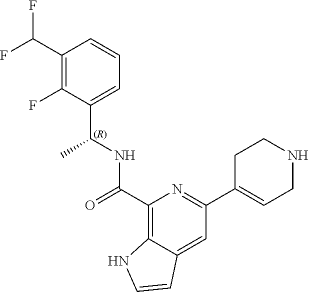

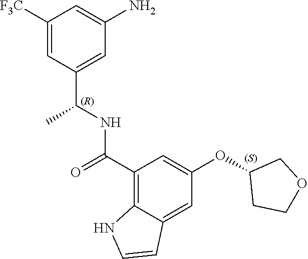

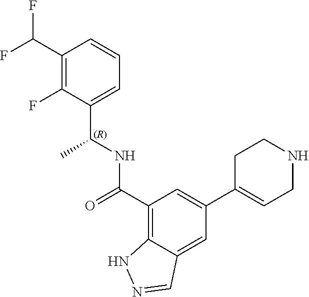

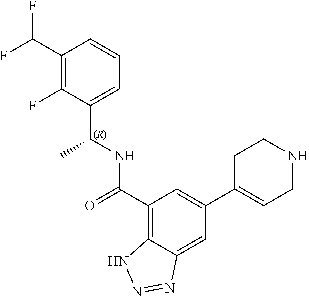

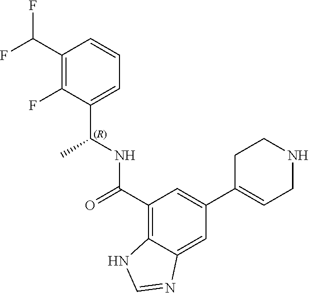

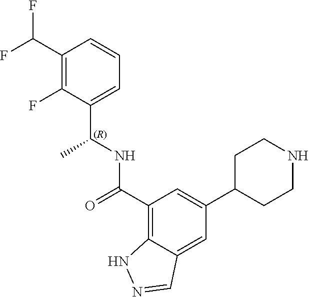

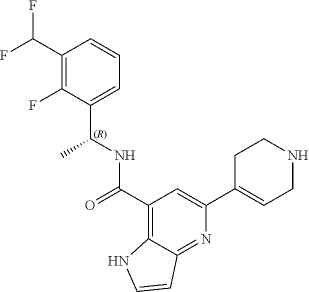

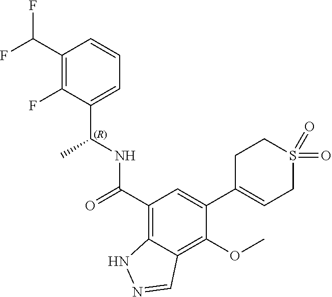

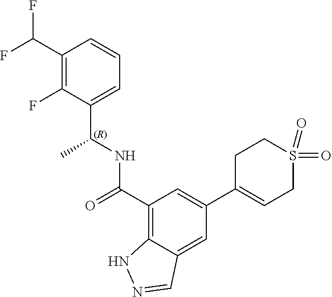

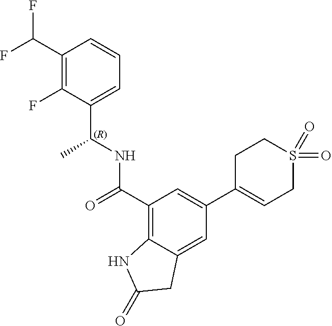

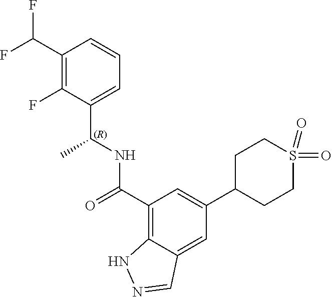

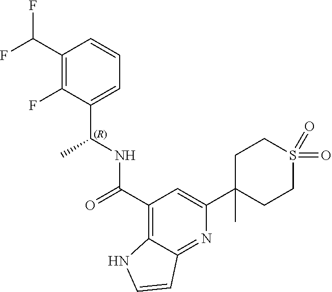

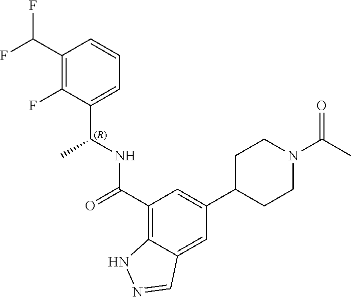

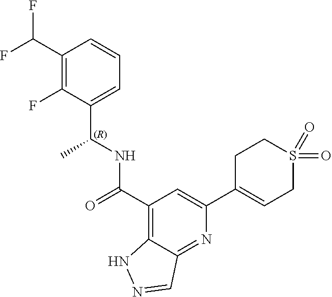

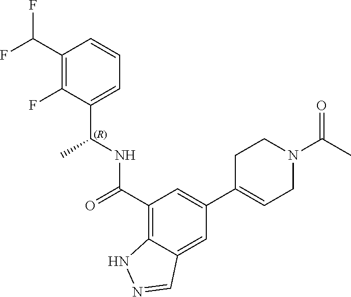

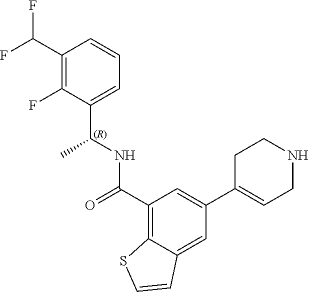

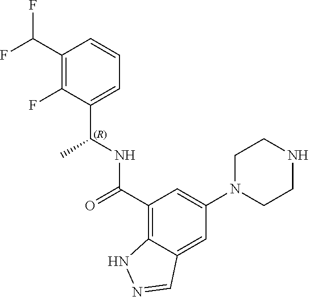

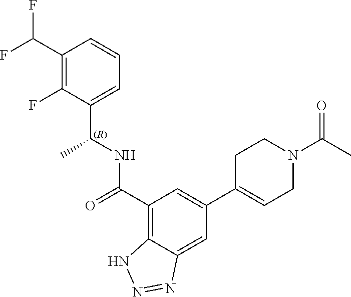

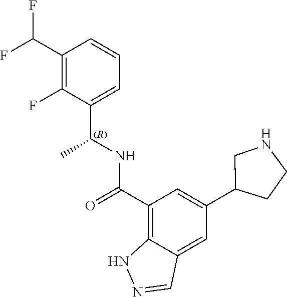

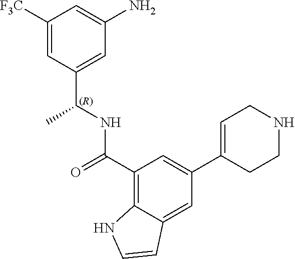

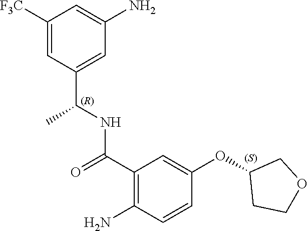

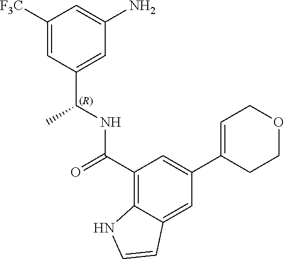

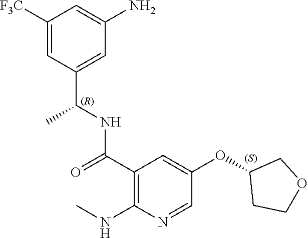

In some embodiments, the SOS1 inhibitor is BI-3406, having the structure:

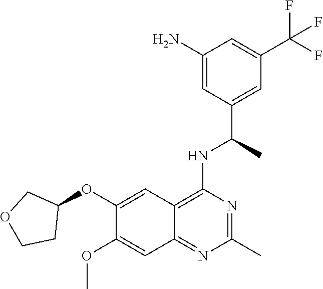 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer (e.g., stereoisomer), prodrug, or tautomer thereof.

In some embodiments, the SOS1 inhibitor is BI-1701963 or a pharmaceutically acceptable salt, solvate, isomer (e.g., stereoisomer), prodrug, or tautomer thereof.

In some embodiments, the SOS1 inhibitor is BAY-293, having the structure:

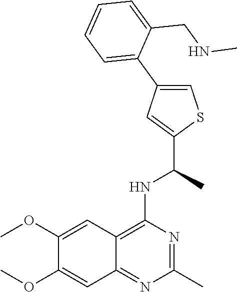

or a pharmaceutically acceptable salt, solvate, isomer (e.g., stereoisomer), prodrug, or tautomer thereof.

In some embodiments, the SOS1 inhibitor is SDGR5 or a pharmaceutically acceptable salt, solvate, isomer (e.g., stereoisomer), prodrug, or tautomer thereof.

In some embodiments, the SOS1 inhibitor is Compound SOS1-(A) (also called RMC-0331), having the structure:

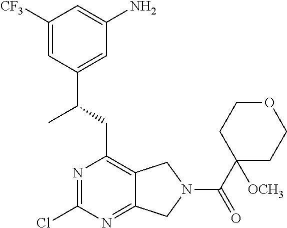 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer (e.g., stereoisomer), prodrug, or tautomer thereof.

In some embodiments, the SOS1 inhibitor is Compound SOS1-(B), or a pharmaceutically acceptable salt, solvate, isomer (e.g., stereoisomer), prodrug, or tautomer thereof.

The SOS1 inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The SOS1 inhibitor can be dosed once per day, twice per day, three times per day, or four times per day. In some aspects, SOS1 inhibitor is dosed once per day. In some aspects, SOS1 inhibitor is dosed twice per day. Dosing may be done with or without food. The dosing schedule may suitably be every day of a 28-day schedule, or 21 or more days of a 28-day schedule.

Mutations in SHP2, e.g., activating SHP2 mutations, may induce RAS/MAPK signaling pathway reactivation and drug resistance to a SHP2 inhibitor in a patient administered a SHP2 inhibitor, e.g., an allosteric SHP2 inhibitor, in the treatment of a tumor or cancer. The present invention is suitable for the treatment of a patient who has a cancer characterized by a mutation of SHP2, e.g., an activating SHP2 mutation, and has therefore developed drug resistance to a SHP2 inhibitor, e.g., an allosteric SHP2 inhibitor. Empirical evidence to date suggests that SHP2 mutations, e.g., activating SHP2 mutations, increase dependence on SOS1 and may therefore increase sensitivity to SOS1 inhibitors such as BI-3406, BI-1701963, and Compound SOS1-(A) (also called RMC-0331), or a pharmaceutically acceptable salt, solvate, isomer (e.g., stereoisomer), prodrug, or tautomer thereof. Accordingly, the therapeutic strategy according to the method of the present invention is directed to the effective treatment of cancer patients with activating mutations in SHP2 by the administration of a therapeutically effective amount of SOS1 inhibitor.

According to embodiments of the present invention, the method comprises administering to the subject a therapeutically effective amount of a SOS1 inhibitor in the treatment of a tumor or cancer. In some embodiments, the disease or disorder is selected from the group consisting of tumors of hematopoietic and lymphoid system; a myeloproliferative syndrome; a myelodysplastic syndrome; leukemia; acute myeloid leukemia; acute B-lymphoblastic leukemia-lymphoma; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma. In some embodiments, the disease or disorder is selected from brain glioblastoma, lung adenocarcinoma, colon adenocarcinoma, bone marrow leukemia, acute myelocytic leukemia (AML), breast carcinoma, unknown primary melanoma, non-small cell lung carcinoma (NSCLC), skin melanoma, breast invasive ductal carcinoma, lung squamous cell carcinoma, unknown primary adenocarcinoma, bone marrow multiple myeloma, gastroesophageal junction adenocarcinoma, bone marrow myelodysplastic syndrome, prostate acinar adenocarcinoma, bladder urothelial (transitional cell) carcinoma, uterus endometrial adenocarcinoma, bone marrow leukemia B cell acute, acute B-lymphoblastic leukemia-lymphoma, stomach adenocarcinoma, and unknown primary carcinoma. In some embodiments, the disease or disorder is selected from the group consisting of AML, lung adenocarcinoma, non-small cell lung carcinoma, brain glioblastoma, a myelodysplastic syndrome, skin melanoma, breast carcinoma, stomach adenocarcinoma, acute B-lymphoblastic leukemia-lymphoma, and colon adenocarcinoma.

In some embodiments, the disease or disorder is acute myelocytic leukemia (AML). In some embodiments, the disease or disorder is AML and the SHP2 mutation is at a position selected from the group consisting of G60, D61, A⁷², E76, G503 and S502, and a combination thereof, and the method optionally further comprises administering to a subject a therapeutically effective amount of a SHP2 inhibitor (e.g., RMC-4630 or a pharmaceutically acceptable salt, solvate, isomer (e.g., stereoisomer), prodrug, or tautomer thereof) and a RAS(ON) inhibitor, such as a RAS(ON)^{G12C} inhibitor of Appendix B (e.g., a compound of Table 1 or Table 2 therein) or a RAS(ON) MULTI inhibitor of Appendix A (e.g., a compound of Table 1 or Table 2 therein). See, e.g., Alfayez et al., Leukemia 35:691-700 (2021).

In some embodiments, the disease or disorder is selected from the group consisting of AML, lung adenocarcinoma, non-small cell lung carcinoma, brain glioblastoma, a myelodysplastic syndrome, skin melanoma, breast carcinoma, stomach adenocarcinoma, acute B-lymphoblastic leukemia-lymphoma, and colon adenocarcinoma, the SHP2 mutation is at a position selected from the group consisting of G60, D61, E69, A⁷², E123, Y¹⁹⁷, N308, V428, A⁴⁶¹, T468, S502, G503, T507 (e.g., A⁷², E76 or G503; or, e.g., G60V, D61G, D61V, D61Y, E69K, E69Q, A⁷²S, A⁷²T, A⁷²V, E123D, N308D, V428M, A⁴⁶¹T, A⁴⁶¹G, T468M, S502L, S502P, G503A, G503V, T507K), and the method optionally further comprises administering to a subject a therapeutically effective amount of a SHP2 inhibitor (e.g., RMC-4630 or a pharmaceutically acceptable salt, solvate, isomer (e.g., stereoisomer), prodrug, or tautomer thereof) and a RAS(ON) inhibitor, such as a RAS(ON)^{G12C} inhibitor of Appendix B (e.g., a compound of Table 1 or Table 2 therein) or a RAS(ON) MULTI inhibitor of Appendix A (e.g., a compound of Table 1 or Table 2 therein).

Activating SHP2 mutations have been associated with developmental RASopathy pathologies such as Noonan syndrome and Leopard syndrome. SHP2 mutations have also been identified in other RASopathies. According to embodiments of the present invention, the method comprises administering to the subject a therapeutically effective amount of a SOS1 inhibitor in the treatment of a RASopathy. In some embodiments, the RASopathy is selected from the group consisting of Neurofibromatosis type 1, Noonan Syndrome, Noonan Syndrome with Multiple Lentigines, Capillary Malformation-Arteriovenous Malformation Syndrome, Costello Syndrome, Cardio-Facio-Cutaneous Syndrome, Legius Syndrome, and Hereditary gingival fibromatosis. In some embodiments, a RASopathy comprises a SHP2 mutation at a position selected from the group consisting of T52, 156, Y⁶², Y⁶³, E69, K70, E139, L261, R265, N308, T468, M504, Q510 (e.g., T521, 156V, Y⁶²D, Y⁶³D, Y⁶³C, E69K, E69Q, K70R, E139D, L261F, L261H, R265Q, N308D, T468M, M504V, Q510P, Q510H). According to embodiments of the present invention, the method comprises administering to the subject a therapeutically effective amount of a SOS1 inhibitor in the treatment of Noonan syndrome or Leopard syndrome.

### RAS and RAS Mutations

In some embodiments, the method of the present invention further comprises administering to the subject a therapeutically effective amount of a RAS inhibitor selected from the group consisting of a RAS(ON) inhibitor, a RAS(OFF) inhibitor, MRTX1133, or a pharmaceutically acceptable salt, solvate, isomer (e.g., stereoisomer), prodrug, or tautomer thereof, and a combination thereof. In some embodiments, the Ras protein is wild-type, and the RAS inhibitor targets a wild-type RAS protein. In some embodiments, the RAS inhibitor targets KRAS, NRAS, or HRAS. In some embodiments, the RAS inhibitor targets two or more of KRAS, NRAS, or HRAS.

In some embodiments, the RAS inhibitor targets a RAS protein having a mutation. In some embodiments, the RAS inhibitor is a RAS mutant specific inhibitor. In some embodiments, the RAS inhibitor targets a KRAS mutant, a NRAS mutant, or an HRAS mutant. In certain embodiments, RAS mutant is selected from:
(a) the following K-Ras mutants: G12D, G12V, G12C, G13D, G12R, G12A, Q61H, G12S, A¹⁴⁶T, G13C, Q61L, Q61R, K117N, A¹⁴⁶V, G12F, Q61K, L19F, Q22K, V14I, A⁵⁹T, A¹⁴⁶P, G13R, G12L, Y⁹⁶D, or G13V, and combinations thereof;
(b) the following H-Ras mutants: Q61R, G13R, Q61K, G12S, Q61L, G12D, G13V, G13D, G12C, K117N, A⁵⁹T, G12V, G13C, Q61H, G13S, A¹⁸V, D119N, G13N, A¹⁴⁶T, A⁶⁶T, G12A, A¹⁴⁶V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A¹⁴⁶T, G60E, Q61P, A⁵⁹D, E132K, E49K, T50I, A¹⁴⁶V, or A⁵⁹T, and combinations thereof;
or a combination of any of the foregoing (e.g., both K-Ras G12C and K-Ras G13C). In some embodiments, the cancer comprises a Ras mutation selected from the group consisting of G12C, G13C, G12A, G12D, G13D, G12S, G13S, G12V and G13V. In some embodiments, the cancer comprises at least two Ras mutations selected from the group consisting of G12C, G13C, G12A, G12D, G13D, G12S, G13S, G12V and G13V. In some embodiments, the cancer comprises at least a G12C mutation and a Y⁹⁶D mutation. Mutations at these positions may result in RAS-driven tumors.

In some embodiments, the RAS inhibitor targets a wild-type RAS protein. In some embodiments, the Ras inhibitor targets RAS^{amp}. In some embodiments, the RAS protein is KRAS. In some embodiments, the RAS protein is NRAS. In some embodiments, a RAS inhibitor targets both a KRAS protein and an NRAS protein. In some embodiments, the RAS inhibitor targets a RAS protein mutation. In some embodiments, the RAS protein mutation is at a position selected from the group consisting of G12, G13, Q61, A¹⁴⁶, K117, L19, Q22, V14, A⁵⁹, and a combination thereof. In some embodiments, the mutation is at a position selected from the group consisting of G12, G13, and Q61. In some embodiments, the mutation is selected from the group consisting of G12C, G12D, G12A, G12S, G12V, G13C, G13D, Q61K, and Q61L.

### RAS Inhibitors

According to some embodiments of the present disclosure, the method comprises treating a subject having a disease or disorder associated with cells having a SHP2 mutation by administering to the subject (a) a therapeutically effective amount of a SOS1 inhibitor or a pharmaceutically acceptable salt, solvate, isomer (e.g., stereoisomer), prodrug, or tautomer thereof; and (b) a therapeutically effective amount of a RAS inhibitor selected from the group consisting of a RAS(ON) inhibitor, a RAS(OFF) inhibitor, MRTX1133 or a pharmaceutically acceptable salt, solvate, isomer (e.g., stereoisomer), prodrug, or tautomer thereof, and a combination thereof.

In some embodiments, the RAS inhibitor is a RAS(OFF) inhibitor known in the art or disclosed herein. The RAS(OFF) inhibitor may be any one or more of the RAS(OFF) inhibitors disclosed in any one of WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021120890, WO 2021120045, WO 2021119343, WO 2021118877, WO 2021113595, WO 2021107160, WO 2021106231, WO 2021106230, WO 2021104431, WO 2021088458, WO 2021086833, WO 2021085653, WO 2021084765, WO 2021081212, WO 2021058018, WO 2021057832, WO 2021055728, WO 2021041671, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020106640, WO 2020102730, WO 2020101736, WO 2020097537, WO 2020086739, WO 2020018282, WO 2020050890, WO 2020047192, WO 2020035031, WO 2020033413, WO 2020028706, WO 2019241157, WO 2019234405, WO 2019232419, WO 2019227040, WO 2019217933, WO 2019217691, WO 2019217307, WO 2019215203, WO 2019213526, WO 2019213516, WO 2019204442, WO 2019204449, WO 2019204505, WO 2019155399, WO 2019150305, WO 2019137985, WO 2019110751, WO 2019099524, WO 2019055540, WO 2019051291, WO 2018237084, WO 2018218070, WO 2018217651, WO 2018218071, WO 2018218069, WO 2018212774, WO 2018206539, WO 2018195439, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2018011351, WO 2018005678, WO 2017201161, WO 20171937370, WO 2017172979, WO 2017112777, WO 2017106520, WO 2017096045, WO 2017100546, WO 2017087528, WO 2017079864, WO 2017058807, WO 2017058805, WO 2017058728, WO 2017058902, WO 2017058792, WO 2017058768, WO 2017058915, WO 2017015562, WO 2016179558, WO 2016176338, WO 2016168540, WO 2016164675, WO 2016100546, WO 2016049568, WO 2016049524, WO 2015054572, WO 2014152588, WO 2014143659 and WO 2013155223, or a pharmaceutically acceptable salt, solvate, isomer (e.g., stereoisomer), prodrug, or tautomer thereof, each of which is incorporated herein by reference in its entirety. In some embodiments, the RAS(OFF) inhibitor is selected from sotorasib (AMG 510), adagrasib (MRTX849), MRTX1257, JNJ-74699157 (ARS-3248), LY³⁵³⁷⁹⁸², ARS-853, ARS-1620, GDC-6036, BPI-421286, JDQ443, and JAB-21000, or a pharmaceutically acceptable salt, solvate, isomer (e.g., stereoisomer), prodrug, or tautomer thereof. In some embodiments, the RAS(OFF) inhibitor selectively targets RAS G12C.

In some embodiments, the compositions and methods described herein utilize a RAS inhibitor that is a RAS(ON) inhibitor known in the art or disclosed herein. In some embodiments, the RAS inhibitor is a RAS(ON) inhibitor. In some embodiments, the RAS(ON) inhibitor is an inhibitor selective for RAS G12C, RAS G13D, or RAS G12D. In some embodiments, the RAS(ON) inhibitor is a RAS(ON) MULTI inhibitor.

The RAS(ON) inhibitor may be any one or more of the RAS(ON) inhibitors disclosed in WO 2020/132597. or a pharmaceutically acceptable salt, solvate, isomer (e.g., stereoisomer), prodrug, or tautomer thereof, or any one of Appendices A, B, C, or D, or a compound described by a Formula of any one of Appendices A, B, C, or D, or a pharmaceutically acceptable salt, solvate, isomer (e.g., stereoisomer), prodrug, or tautomer thereof.

In some embodiments, the RAS inhibitor is a compound disclosed in Appendix A. In some embodiments, the RAS(ON) inhibitor is a compound described by Formula I in Appendix A, or a pharmaceutically acceptable salt, solvate, isomer (e.g., stereoisomer), prodrug, or tautomer thereof. In some embodiments, the RAS(ON) inhibitor is selected from a compound of Table 1 or Table 2 of Appendix A, or a pharmaceutically acceptable salt, solvate, isomer (e.g., stereoisomer), prodrug, or tautomer thereof.

In some embodiments, the RAS inhibitor is a compound disclosed in Appendix B. In some embodiments, the RAS(ON) inhibitor is a compound described by Formula I in Appendix B, or a pharmaceutically acceptable salt, solvate, isomer (e.g., stereoisomer), prodrug, or tautomer thereof. In some embodiments, the RAS(ON) inhibitor is selected from a compound of Table 1 or Table 2 of Appendix B, or a pharmaceutically acceptable salt, solvate, isomer (e.g., stereoisomer), prodrug, or tautomer thereof.

In some embodiments, the RAS inhibitor is a compound disclosed in Appendix C. In some embodiments, the RAS(ON) inhibitor is a compound described by Formula I in Appendix C, or a pharmaceutically acceptable salt, solvate, isomer (e.g., stereoisomer), prodrug, or tautomer thereof. In some embodiments, the RAS(ON) inhibitor is selected from a compound of Table 1 or Table 2 of Appendix C, or a pharmaceutically acceptable salt, solvate, isomer (e.g., stereoisomer), prodrug, or tautomer thereof.

In some embodiments, the RAS inhibitor is a compound disclosed in Appendix D. In some embodiments, the RAS(ON) inhibitor is a compound described by Formula I in Appendix D, or a pharmaceutically acceptable salt, solvate, isomer (e.g., stereoisomer), prodrug, or tautomer thereof. In some embodiments, the RAS(ON) inhibitor is selected from a compound of Table 1 or Table 1-1 of Appendix D, or a pharmaceutically acceptable salt, solvate, isomer (e.g., stereoisomer), prodrug, or tautomer thereof.

In some embodiments, the RAS inhibitor is selected from the group consisting of Compound RAS-(A) (a RAS(ON)^{G12C} inhibitor of Appendix B), Compound RAS-(B) (a RAS(ON)^{G12C} inhibitor of Appendix B), Compound RAS-(C) (a RAS(ON)^{G13C} inhibitor of Appendix B), Compound RAS-(D) (a RAS(ON)_{MULTI} inhibitor of Appendix A), Compound RAS-(E) (RAS(ON)_{MULTI} inhibitor of Appendix D), or a pharmaceutically acceptable salt, solvate, isomer (e.g., stereoisomer), prodrug, or tautomer thereof, and a combination thereof. It is to be understood that any one of Compound RAS-(A), Compound RAS-(B), Compound RAS-(C), Compound RAS-(D), Compound RAS-(E) could be found in any one of Appendices A, B, C, and D. Accordingly, the letter reference to the RAS compound (e.g., RAS-(A)) should not be understood to necessarily indicate that the compound can be found in the corresponding Appendix (e.g., Appendix A).

In some embodiments, the RAS inhibitor is Compound RAS-(A), or a pharmaceutically acceptable salt, solvate, isomer (e.g., stereoisomer), prodrug, or tautomer thereof.

In some embodiments, the RAS inhibitor is Compound RAS-(B), or a pharmaceutically acceptable salt, solvate, isomer (e.g., stereoisomer), prodrug, or tautomer thereof.

In some embodiments, the RAS inhibitor is Compound RAS-(C), or a pharmaceutically acceptable salt, solvate, isomer (e.g., stereoisomer), prodrug, or tautomer thereof.

In some embodiments, the RAS inhibitor is Compound RAS-(D), or a pharmaceutically acceptable salt, solvate, isomer (e.g., stereoisomer), prodrug, or tautomer thereof.

In some embodiments, the RAS inhibitor is Compound RAS-(E), or a pharmaceutically acceptable salt, solvate, isomer (e.g., stereoisomer), prodrug, or tautomer thereof.

In some embodiments, the RAS inhibitor is selective for a mutation at position 12 or 13 of a RAS protein. In some embodiments, the RAS inhibitor selectively targets RAS G12D. In some embodiments, the RAS inhibitor is MRTX1133, or a pharmaceutically acceptable salt, solvate, isomer (e.g., stereoisomer), prodrug, or tautomer thereof.

The RAS inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The RAS inhibitor can be dosed once per day, twice per day, three times per day, or four times per day. In some aspects, RAS inhibitor is dosed once per day. In some aspects, RAS inhibitor is dosed twice per day. Dosing may be done with or without food. The dosing schedule may suitably be every day of a 28-day schedule, or 21 or more days of a 28-day schedule.

### Exemplary Combinations of RAS Inhibitors and SOS1 Inhibitors

In some embodiments, the method comprises administering a combination of a RAS inhibitor and a SOS1 inhibitor. Exemplary, non-limiting combinations of such inhibitors include the following.

In one embodiment, (a) the SOS1 inhibitor is Compound SOS1-(A) (also called RMC-0331), having the structure:

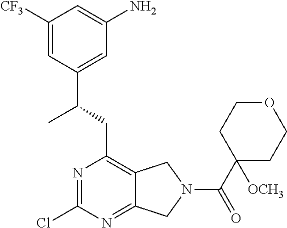 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer (e.g., stereoisomer), prodrug, or tautomer thereof; and (b) the RAS inhibitor is Compound RAS-(C), or a pharmaceutically acceptable salt, solvate, isomer (e.g., stereoisomer), prodrug, or tautomer thereof.

In one embodiment, (a) the SOS1 inhibitor is Compound SOS1-(A) (also called RMC-0331), having the structure:

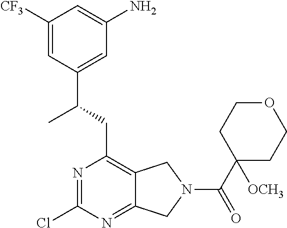 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer (e.g., stereoisomer), prodrug, or tautomer thereof; and (b) the RAS inhibitor is Compound RAS-(D), or a pharmaceutically acceptable salt, solvate, isomer (e.g., stereoisomer), prodrug, or tautomer thereof.

In one embodiment, (a) the SOS1 inhibitor is Compound SOS1-(B), or a pharmaceutically acceptable salt, solvate, isomer (e.g., stereoisomer), prodrug, or tautomer thereof; and (b) the RAS inhibitor is Compound RAS-(B), or a pharmaceutically acceptable salt, solvate, isomer (e.g., stereoisomer), prodrug, or tautomer thereof.

In one embodiment, (a) the SOS1 inhibitor is Compound SOS1-(B), or a pharmaceutically acceptable salt, solvate, isomer (e.g., stereoisomer), prodrug, or tautomer thereof; and (b) the RAS inhibitor is Compound RAS-(E), or a pharmaceutically acceptable salt, solvate, isomer (e.g., stereoisomer), prodrug, or tautomer thereof.

In one embodiment, (a) the SOS1 inhibitor is BI-3406, having the structure:

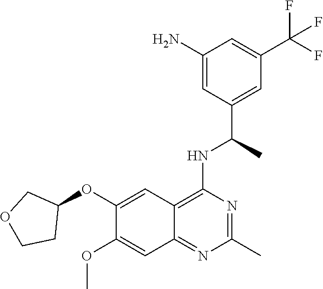 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer (e.g., stereoisomer), prodrug, or tautomer thereof; and (b) the RAS inhibitor is Compound RAS-(A), or a pharmaceutically acceptable salt, solvate, isomer (e.g., stereoisomer), prodrug, or tautomer thereof.

In one embodiment, (a) the SOS1 inhibitor is BI-3406, having the structure:

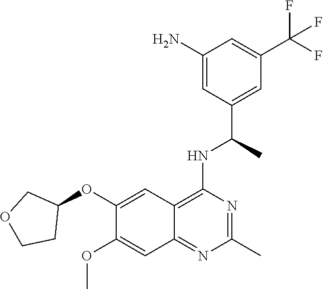 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer (e.g., stereoisomer), prodrug, or tautomer thereof; and (b) the RAS inhibitor is Compound RAS-(C), or a pharmaceutically acceptable salt, solvate, isomer (e.g., stereoisomer), prodrug, or tautomer thereof.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is selected from the group consisting of Compound RAS-(A), Compound RAS-(B), Compound RAS-(C), Compound RAS-(D), Compound RAS-(E), and a combination thereof, or a pharmaceutically acceptable salt, solvate, isomer (e.g., stereoisomer), prodrug, or tautomer of any of the above.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is Compound RAS-(A), or a pharmaceutically acceptable salt, solvate, isomer (e.g., stereoisomer), prodrug, or tautomer thereof.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is Compound RAS-(B), or a pharmaceutically acceptable salt, solvate, isomer (e.g., stereoisomer), prodrug, or tautomer thereof.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is Compound RAS-(C), or a pharmaceutically acceptable salt, solvate, isomer (e.g., stereoisomer), prodrug, or tautomer thereof.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is Compound RAS-(D), or a pharmaceutically acceptable salt, solvate, isomer (e.g., stereoisomer), prodrug, or tautomer thereof.

In some embodiments, (a) the SOS1 inhibitor is BI-1701963 or a pharmaceutically acceptable salt, solvate, isomer (e.g., stereoisomer), prodrug, or tautomer thereof; and (b) the RAS inhibitor is Compound RAS-(E), or a pharmaceutically acceptable salt, solvate, isomer (e.g., stereoisomer), prodrug, or tautomer thereof.

### Additional Combination Therapies

In some embodiments, the subject is co-administered a therapeutically effective amount of an additional therapeutic agent. With respect to any compound in this Additional Combination Therapies section, it is to be understood that a pharmaceutically acceptable salt, solvate, isomer (e.g., stereoisomer), prodrug, or tautomer thereof is also contemplated.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A²aR, B-7 family ligands, or a combination thereof. In some embodiments, the checkpoint inhibitor is pembrolizumab, nivolumab, cemiplimab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A³¹⁷ (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514, MEDI0680, BMS936559, MED14736, MPDL3280A, MSB0010718C, BMS986016, IMP321, lirilumab, IPH2101, 1-7F9, and KW-6002. In some embodiments, the PD-1 inhibitor may be JTX-4014, spartalizumab, camrelizumab, sintilimab, tislelizumab, toripalimab, dostarlimab, INCMGA⁰⁰⁰¹², AMP-224 or AMP-514.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog.

Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gamma1l and calicheamicin omega1l (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris (2-chloroethyl) amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK 4/6 inhibitor such as ribociclib, abemaciclib, or palbociclib), seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP (Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY²¹²⁷³⁹⁹), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, the anti-cancer agent is a colony-stimulating factor 1 receptor (CSF1R) inhibitor. See, e.g., Cannarile et al., J ImmunoTherapy Cancer 5:53 (2017) and Xun et al., Curr Med Chem 27:3944 (2020).

In some embodiments, an anti-cancer agent is an anti-CD40 antibody, such as APX005M.

A therapeutic agent may be an anti-TIGIT antibody, such as MBSA⁴³, BMS-986207, MK-7684, COM902, AB154, MTIG7192A or OMP-313M32 (etigilimab).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, RLY-1971, ERAS-601), another SOS1 inhibitor (e.g., BI-1701963), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312. In some embodiments, an anti-cancer agent is a Ras inhibitor (e.g., AMG 510, MRTX1257, JNJ-74699157 (ARS-3248), LY³⁵³⁷⁹⁸², ARS-853, ARS-1620, GDC-6036, BPI-421286, JDQ443 or JAB-21000), or a Ras vaccine, or another therapeutic modality designed to directly or indirectly decrease the oncogenic activity of Ras.

In some embodiments, a therapeutic agent is an inhibitor of the MAP kinase (MAPK) pathway (or “MAPK inhibitor”). MAPK inhibitors include, but are not limited to, one or more MAPK inhibitor described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH₄₉₈₇₆₅₅); CI-1040; PD-0325901; CH₅₁₂₆₇₆₆; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5): 989-1000).

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7 (3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15: 59 (8): 1935-40; and Yang et al., Cancer Res.1999, 59:1236-1243. The EGFR inhibitor can be monoclonal antibody Mab E7.6.3 (Yang, 1999 supra), or Mab C₂₂₅ (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39 (4): 565-8; and Paez et al., EGFR Mutations In Lung Cancer Correlation With Clinical Response To Gefitinib Therapy, Science 2004, 304 (5676): 1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8 (12): 1599-1625. In some embodiments, an EGFR inhibitor is osimertinib.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo [4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY²⁹⁴⁰⁰² (2-(4-morpholinyl)-8-phenyl-4H-1-benzopyran-4-one (available from Axon Medchem); PI 103 hydrochloride (3-[4-(4-morpholinylpyrido-[3′, 2′: 4,5]furo [3,2-d]pyrimidin-2-yl]phenol hydrochloride (available from Axon Medchem); PIK 75 (2-methyl-5-nitro-2-[(6-bromoimidazo [1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo [1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino) ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Akt1) (Barnett et al., Biochem. J. 2005, 385 (Pt. 2): 399-408); Akt-1-1,2 (inhibits Ak1 and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo [4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g., AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A⁷⁶²E.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA⁴, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110 (1): 186-192; Thompson et al., Clin. Cancer Res. 2007, 13 (6): 1757-1761; and WO06/121168 A¹), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A⁶, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A⁴ prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY²⁰⁴⁰⁰², N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA²⁷¹; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments, an additional compound is selected from the group consisting of a CDK4/6 inhibitor (e.g., abemaciclib, palbociclib, or ribociclib), a KRAS: GDP G12C inhibitor (e.g., AMG 510, MRTX 1257) or other mutant Ras: GDP inhibitor, a KRAS: GTP G12C inhibitor or other mutant Ras: GTP inhibitor, a MEK inhibitor (e.g., refametinib, selumetinib, trametinib, or cobimetinib), a SHP2 inhibitor (e.g., TNO155, RMC-4630), an ERK inhibitor, and an RTK inhibitor (e.g., an EGFR inhibitor). In some embodiments, a SOS1 inhibitor may be used in combination with a Ras inhibitor, a SHP2 inhibitor, or a MEK inhibitor. In some embodiments, a combination therapy includes a SOS1 inhibitor, a RAS inhibitor and a MEK inhibitor.

In some embodiments, an additional compound is selected from the group consisting of ABT-737, AT-7519, carfilzomib, cobimetinib, danusertib, dasatinib, doxorubicin, GSK-343, JQ1, MLN-7243, NVP-ADW742, paclitaxel, palbociclib and volasertib. In some embodiments, an additional compound is selected from the group consisting of neratinib, acetinib and reversine.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In the above, preferred additional therapeutic agents include MEK inhibitors, ERK inhibitors, pan-RAS(ON) inhibitors (that is, inhibitors that target the GTP-activated form of RAS), CDK4/6 inhibitors, mTORC1 inhibitors, HDAC inhibitors, BCL2 inhibitors, and PLK1 inhibitors.

In some embodiments, the method further comprises administering to the subject a therapeutically effective amount of an AML therapeutic agent. Such agents are known in the art, and may be selected from, e.g., cytarabine, an anthracycline drug (e.g., daunorubicin or idarubicin), midostaurin, gemtuzumab ozogamicin, cladribine, fludarabine, etoposide, azacytidine, decitabine, venetoclax, glasdegib, ivosidenib, or enasidenib, or a combination thereof.

In some embodiments, the present disclosure provides a method for patient stratification based upon the presence or absence of a SHP2 mutation, in particular an activating SHP2 mutation. As used herein, “patient stratification” means classifying one or more patient as having a disease or disorder (e.g., cancer) that is either likely or unlikely to be treatable with a SHP2 inhibitor, such as an allosteric SHP2 inhibitor. Patient stratification may comprise classifying a patient as having a tumor that is sensitive or resistant to treatment with a SHP2 inhibitor, such as an allosteric SHP2 inhibitor.

In some embodiments, the method of the present invention comprises identifying the subject as resistant to SHP2 inhibitor, such as an allosteric SHP2 inhibitor, by genotyping a biological sample from the subject for a SHP2 mutation, wherein the subject is identified as resistant to the SHP2 inhibitor if the SHP2 mutation comprises an inhibitor-resistant mutation, such as an allosteric inhibitor-resistant mutation.

For example, but not to be limited in anyway, in some aspects, a biological sample from a patient (e.g., a cell such as a tumor cell) may be genotyped using a hybridization detection method to determine whether the cell contains a SHP2 mutation, such as an activating SHP2 mutation, comprising an inhibitor-resistant mutation, such as an allosteric inhibitor-resistant mutation.

Hybridization detection methods are based on the formation of specific hybrids between complementary nucleic acid sequences that serve to detect nucleic acid sequence mutation(s). Such methods include, e.g., microarray analysis and real time PCR. Hybridization methods, such as Southern analysis, Northern analysis, or in situ hybridizations, may also be used (see Current Protocols in Molecular Biology, Ausubel et al., eds., John Wiley & Sons 2003, incorporated by reference in its entirety).

Other suitable methods for genotyping a cell (e.g., a tumor cell) include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8 (4): 387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In some embodiments, the method of the present invention comprises performing a diagnostic test to determine whether the subject has a SHP2 mutation that induces an activated form of SHP2.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Methods Used in the Examples

### CrownSyn™ Methods (3-Dimensional Surface Plots)

Cells were grown in 3-dimensional culture in the appropriate growth medium containing 0.65% methylcellulose. On the day of cell seeding, the cells were harvested from 2-dimensional culture during the logarithmic growth period, mixed with appropriate cell media and centrifuged at 1000 rpm for 4 minutes. Cells were re-suspended and counted using CountStar. 3.5 mL of cell suspension was mixed with 6.5 mL of 1% methylcellulose, yielding 10 ml of cell suspension in 0.65% methylcellulose solution. 90 μL cell suspension was added to 96-well plates. Another plate prepared for T0 reading. Plates were incubated in humidified incubator at 37° C. with 5% CO₂. Test articles were diluted using DMSO or culture medium to 10× working solution. 10 μl each test article solution was dispensed separately to each well (triplicate for each concentration). Plates were cultured for 120 hr in humidified incubator at 37° C. with 5% CO₂ or 100% air. For TO reading, 10 μl culture medium was added to each well of T0 plate, and cell viability determined using CTG assay as described below. After 120 hours, plates were equilibrated at room temperature for approximately 30 minutes, prior to addition of 100 μl of CellTiter-Glo® Reagent into each assay well. Contents mixed for 2 minutes on an orbital shaker to induce cell lysis. Plates were allowed to incubate at room temperature for 10 minutes to stabilize luminescent signal. Luminescence was recorded using EnVision MultiLabel Reader. The software of GraphPad Prism used to calculate IC₅₀. The graphical curves were fitted using a nonlinear regression model with a sigmoidal dose response. Two dimensional concentration-response surfaces were compared to two different additivity models, Bliss independence and Loewe additivity. Deviation from the prediction of either model was assessed in the form of a synergy score. Synergy scores greater than 5 with either model and at any point on the concentration response surface were interpreted as indicating a significant interaction between compounds. See Bliss C.I. (1939) The toxicity of poisons applied jointly. Ann. Appl. Biol., 26, 585-615, and see Loewe S. (1953) The problem of synergism and antagonism of combined drugs. ArzneimittelForschung, 3,2 86-290. The Bliss independence model is expected to hold true for non-interacting drugs that elicit their responses independently, e.g., by targeting separate pathways. Loewe additivity, in contrast, is more compatible with the cases where both drugs have similar modes of action on the same targets or pathways. As pointed out in the Saariselka agreement (Greco et al., 1992), and also by many others, neither Loewe additivity nor Bliss independence is necessarily reflecting the expected modes of action of a drug combination. Rather, Loewe and Bliss models should be used as data exploratory approaches, with a major purpose to identify potential synergistic drug combinations that warrant further mechanistic investigation, but not the other way around, i.e., using the mechanistic evidence to determine which reference model is more appropriate.

### 2-Dimensional Potency Shifts

Cells were grown in 2-dimensional culture. Assay stocks were thawed and diluted in the recommended ATCC medium, supplemented with 10% serum and 1% pen/strep (final concentration) and dispensed in a 384-well plate. Depending on the cell line used, a cell density of 100-6400 cells per well in 45 μl medium was used. The margins of the plate were filled with phosphate-buffered saline. Plated cells were incubated in a humidified atmosphere of 5% CO₂ at 37° C. After 24 hours, 5 μl of reference compound dilution, containing the single dose compound at indicated concentration was added to the plates and these were further incubated for 120 hrs. At t=120 hours, 24 μl of ATPlite 1Step™ (PerkinElmer) solution was added to each well, and subsequently shaken for 2 minutes. After 5 minutes incubation in the dark, the luminescence was recorded on an Envision multilabel reader (PerkinElmer). On a parallel t=0 plate, 45 μl cells were dispensed and incubated in a humidified atmosphere of 5% CO₂ at 37° C. After 24 hours 5 μl DMSO-containing Hepes buffer and 24 μl ATPlite 1Step™ solution were mixed, and luminescence measured after 5 minutes incubation (=luminescence t=0). Curves and IC₅₀s were calculated by non-linear regression using IDBS XLfit 5. The percentage growth after incubation until t=end (% growth) was calculated as follows: 100%×(luminescence_{t=end}/luminescence_{untreated,t=end}). This was fitted to the ¹⁰ log compound concentration (conc) by a 4 parameter sigmoidal curve: %-growth=bottom+(top−bottom)/(1+10 (log IC₅₀−conc)*hill)) where hill is the Hill-coefficient, and bottom and top the asymptotic minimum and maximum cell growth that the compound allows in that assay.

### Inhibition of Phospho-ERK

Phospho ERK in cells was determined using the MSD® platform. NCI-H1355 or TOV-21G cells were plated in clear flat-bottom 96-well tissue culture plates at 30,000 cells/well in 100 μL/well in complete media, and placed in incubator (37° C., 5% CO₂) overnight. All compounds were reconstituted in DMSO to reach 1000× of desired top concentration and arrayed in column 1 of a 96-well plate. 3-fold serial dilution performed in DMSO of all compounds across the 96-well plate, leaving column 11 with 100% DMSO and column 12 empty. Compounds were diluted in media 1:500 and mixed well. Dilution series at 2× final concentration were then mixed with 2× single dose compounds or DMSO vehicle. Cell media was aspirated from cell plates, and 100 μL of 1× compound mixture was added. Plates were placed in incubator (37° C., 5% CO₂) for 4 hours. MSD® lysis buffer was prepared immediately before time point by mixing 10 mL Tris Lysis Buffer (provided in MSD® kit), 1 tablet PhosSTOP EASYpack (Roche), 1 tablet complete Mini, EDTA-free Protease Inhibitor Cocktail (Roche), 40 μL PMSF (provided in MSD® kit), 100 μL SDS (provided in MSD® kit), and kept on ice before use. After treatment time, media was aspirated from plates and 50 μL MSD® lysis buffer added to each well. Plates were sealed with foil adhesive and shaken for 5 minutes at 750 rpm at room temperature. Plates were then incubated on ice for 15 minutes, and stored at-80° C. MSD® Phospho (Thr202/Tyr204; Thr185/Tyr187)/Total ERK1/2 Assay performed according to manufacturer protocol.

### Example 1. Cells with Activating Mutations in SHP2 have Reduced Sensitivity to Allosteric Inhibitors of SHP2 but Retain Sensitivity to SOS1

Aberrant activation of the RAS/MAPK signaling pathway (shown schematically in FIG. 1) is a common driver of abnormal growth and proliferation in many types of cancer. SHP2 acts near the top of this pathway, responding to inputs from growth factor receptors to recruit and activate the RAS GEFs SOS1 and SOS2. Allosteric inhibitors of SHP2 such as RMC-4550 can block pathway activation and cancer cell growth by interfering with this process. Mutations can occur in SHP2 that uncouple growth factor receptor signaling from SHP2 activation, leading to hyper-activation of pathway signaling. These mutations act by destabilizing an auto-inhibited conformation of SHP2. Different activating mutations destabilize this conformation to different degrees, which can be expressed quantitatively as the free energy of opening (ΔGₒₚ) of the mutation. Wild-type SHP2 has a ΔGₒₚ of 2.8 kcal/mol. Values of ΔGₒₚ below 2.8 in mutant SHP2 indicate activation, with lower values indicating stronger activation. The strength of activation of a SHP2 mutation is correlated with reduced sensitivity to SHP2 allosteric inhibitors such as RMC-4550 in engineered HEK293 cells. See FIG. 2A, which is a graph correlating the RMC-4550 pERK IC₅₀ as a function of ΔGₒₚ. FIG. 2B is a table showing the pERK IC₅₀ values for RMC-4550 in a variety of activating mutations of SHP2 protein.

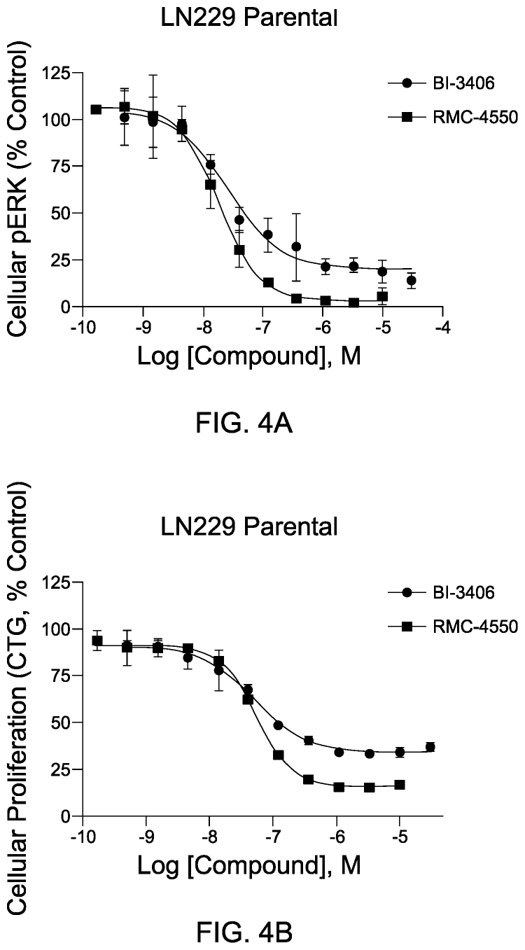

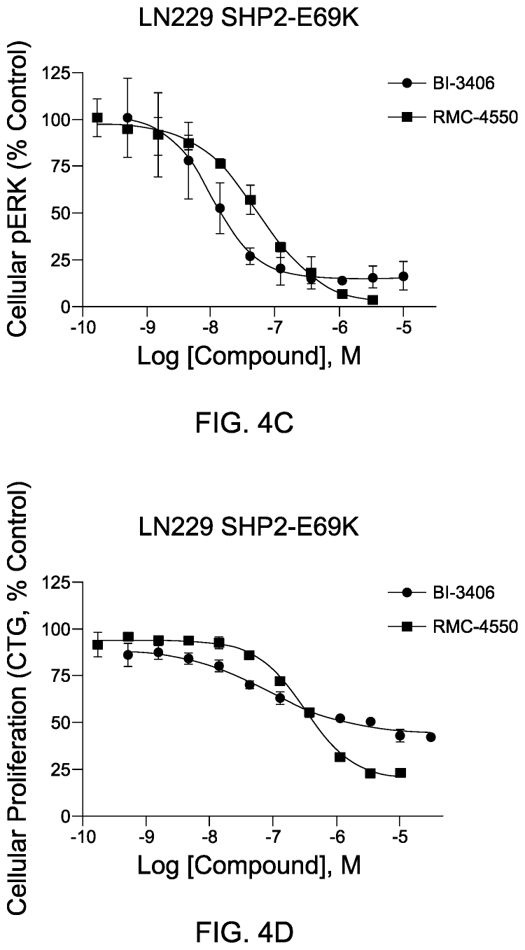

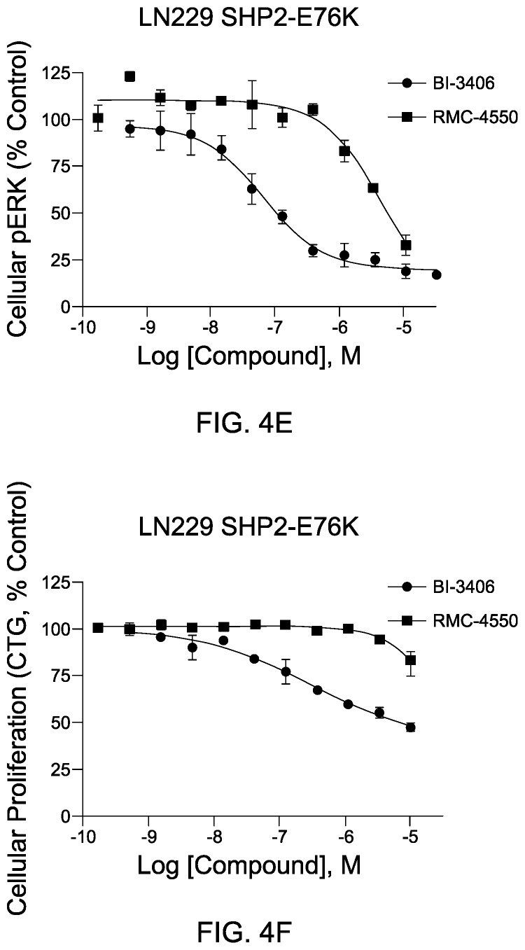

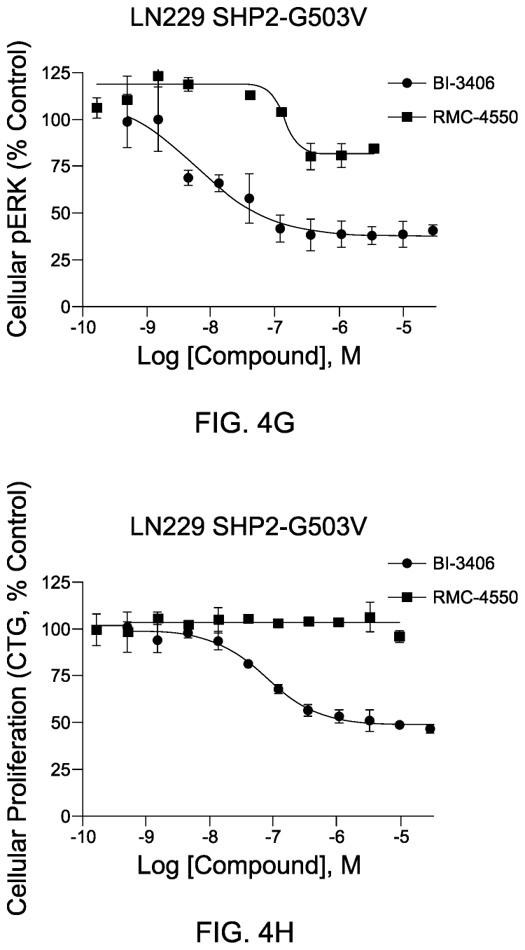

Unlike allosteric SHP2 inhibitors, SOS1 inhibitors are effective in suppressing the RAS/MAPK signaling pathway activation in HEK293 cells with a variety of SHP2 mutations, even in cases where SHP2 inhibition is not effective. See FIGS. 3A,3B, 20A, and 20B, which show that SOS1 inhibitors maintain sensitivity across a broad panel of SHP2 mutant variant contexts in isogenic HEK-293 cell lines. Growth of HEK293 cells is not dependent on RAS/MAPK signaling pathway activation, but growth of LN-229 cells is. FIGS. 4A through 4H show that SOS1 inhibitor suppressed growth of LN229 cells with activating mutations in SHP2 more potently than the allosteric SHP2 inhibitor RMC-4550, with a greater benefit for SOS1 inhibition associated with more strongly activating SHP2 mutations.

The following methods were used to obtain the data shown in FIGS. 2A, 2B, 3A, 3B, 4A through 4H, 20A, and 20B.

### Generation of Isogenic SHP2 Expression Cell Lines

An experimental system was created to test the activity of SHP2 mutants on an isogenic background. See FIGS. 2A, 2B, 3A, 3B, 20A, and 20B. The Flp-In T-REX-293 cell line was obtained from Gibco and cultivated in high glucose DMEM™ containing 2 mM L-glutamine (Hyclone), supplemented with 10% FBS (Hyclone), 1% penicillin/streptomycin (Gibco), 100 μg/mL Zeocin™ (Gibco), and 15 μg/mL blasticidin (Gibco) in a humidified cell culture incubator at 37° C., 5% CO₂.

Wild type or mutant SHP2 variants were synthesized and subcloned into the pcDNA⁵/FRT/TO vector (ThermoFisher). Plasmids were co-transfected with the pOG44 Flp recombinase expression plasmid (ThermoFisher) into Flp-In T-REX-293 cells using X-tremegene 9 DNA transfection reagent (Sigma), according to the manufacturer's instructions. Cells that underwent successful recombination were selected in high glucose DMEM containing 2 mM L-glutamine, supplemented with 10% FBS and, 1% penicillin/streptomycin, 200 μg/mL hygromycin B (Gibco), and 15 μg/mL blasticidin (Gibco) (recombinant selection media) in a humidified cell culture incubator at 37° C., 5% CO₂, until colonies were visually discernible. Colonies were expanded in recombinant selection media in a humidified cell culture incubator at 37° C., 5% CO₂ to establish isogenic SHP2 variant expression cell lines (T-REX-293-SHP2).

### Determination of Sensitivity to SOS Inhibitors

One day prior to compound treatment, T-REx-293-SHP2 cells for each tested variant were harvested and seeded in high glucose DMEM containing 2 mM L-glutamine, supplemented with 0.1% FBS and, 1% penicillin/streptomycin, 200 μg/mL hygromycin B, and 15 μg/mL blasticidin in 96-well assay plates at a density of 20,000 cells/well. Expression of SHP2 constructs was induced by the addition of doxycycline (final concentration=0.1 μg/mL) (Sigma) for 24 hours.

On the day of the experiment, cells were incubated in quadruplicate wells in the presence of increasing concentrations of BI-3406, Compound SOS1-(A) (also called RMC-0331), RMC-4550 (0.17 nM to 10 μM final assay concentration), or vehicle (final assay concentration 0.1% DMSO) at 37° C., 5% CO₂ for 1 hour. For the final 5 minutes of drug treatment, cells were stimulated with 50 ng/ml Epidermal Growth Factor (Sigma). After this incubation was complete, media was aspirated and cellular lysates prepared using lysis buffer provided with the AlphaLISA® detection kit (PerkinElmer). ERK1/2 phosphorylation at Thr202/Tyr204 was assayed using the AlphaLISA® SureFire® Ultra™ HV pERK Assay Kit (Perkin Elmer) following the manufacturer's instructions. Samples were read using an EnVision® Multilabel Plate Reader (Perkin Elmer) using standard AlphaLISA® settings. Assay data was plotted and EC50 values were determined using four-parameter concentration-response model in GraphPad Prism 7. Data provided are mean+/−standard deviation of duplicate values from representative experiments.

### Generation of Isogenic LN229 SHP2 Mutant Cell Lines

An experimental system was created to test the activity of SHP2 mutants on an isogenic background. See FIGS. 4A through 4H. SHP2 mutations were introduced with Synthego CRISPR editing techniques, and LN229 variant cell lines stably expressing these mutations were generated.

Determination of Sensitivity to SOS1 and SHP2 Inhibitors by pERK Alphalisa

One day prior to treatment, LN229 cell lines with each indicated mutation were harvested and seeded in high glucose DMEM containing 2 mM L-glutamine, supplemented with 10% FBS in 96-well assay plates at a density of 30,000 cells/well.

Cell medium was aspirated, and 50 μL standard cell culture medium was added (DMEM, 10% FBS). 50 μL of 2× stock of compounds prepared from DMSO dilution series with 0.2% DMSO added to yield final DMSO concentration of 0.1% (i.e. 1:1000 final dilution of DMSO stock in standard medium). Plate returned to incubator for 1 hours for pERK AlphaLISA®.

After this incubation was complete, media was aspirated and cellular lysates prepared using lysis buffer provided with the AlphaLISA® detection kit (PerkinElmer). ERK1/2 phosphorylation at Thr202/Tyr204 was assayed using the AlphaLISA® SureFire® Ultra™ HV PERK Assay Kit (Perkin Elmer) following the manufacturer's instructions. Samples were read using an EnVision® Multilabel Plate Reader (Perkin Elmer) using standard AlphaLISA® settings. Assay data was plotted and EC50 values were determined using four-parameter concentration-response model in GraphPad Prism 7. Data provided are mean+/−standard deviation of duplicate values from representative experiments.

### Determination of Sensitivity to SOS1 and SHP2 Inhibitors by 3D CellTiter-Glo®

On day 0, cells seeded (100 μL per well for 96-well) at 5,000 cells/well in 96-well ultra-low attachment (ULA) plates (Corning #7007). Immediately after seeding, ULA plate centrifuged for 10 minutes, 1000 RPM, at room temperature, prior to incubation at 37° C., 5% CO₂ overnight.

On day 1, wells visually inspected to confirm spheroid formation. Incubation continued until Day 3.

On day 3, ULA plate removed from the incubator and 10 μL of fresh culture media containing test compound at 11× the desired final concentration added. Plates placed in incubator at 37° C., 5% CO₂ until endpoint assay (Day 7-8).

On day 8, an ATP endpoint viability assay (CellTiter-Glo® 3D, Promega #G9681) performed, following the manufacturer's instructions. CellTiter-Glo® 3D reagent added at a volume equal to the volume of cell culture medium present in each well (100 μL). Plates were shaken for 5 minutes. Contents mixed by carefully pipetting up and down 10 times until spheroids are fully dissociated.

Lysates were transferred into solid white flat-bottom plates (Corning #3917), and incubated for an additional 25 minutes at room temperature. Luminescence measured on a SpectraMax Microplate Reader.

### Results

Fifteen stable, isogenic cell lines in HEK-293 background expressing different SHP2 variants were created using the FRT/TO system. Cells were incubated with Compound SOS1-(A) (also called RMC-0331), BI-3406, or RMC-4550 prior to stimulation with epidermal growth factor (EGF) and measurement of cellular pERK levels by AlphaLISA®. See FIGS. 2A, 2B, 3A, 3B, 20A, and 20B. RMC-4550 potency for inhibition of mutants in cellular context correlated with biochemical potency for activated SHP2 variant. See FIGS. 2A and 2B.

Four isogenic LN229 cell lines expressing different stable SHP2 variants were generated. Activating mutations induce resistance to allosteric SHP2 inhibition, but maintain sensitivity to SOS1 inhibitors in isogenic LN229 cell lines. See FIGS. 4A through 4H.

### Example 2. SOS1 Inhibitors Inhibit Pathway Signaling and Cell Growth In Vitro, and Tumor Growth In Vivo in Tumor Models Bearing SHP2 Activating Mutations

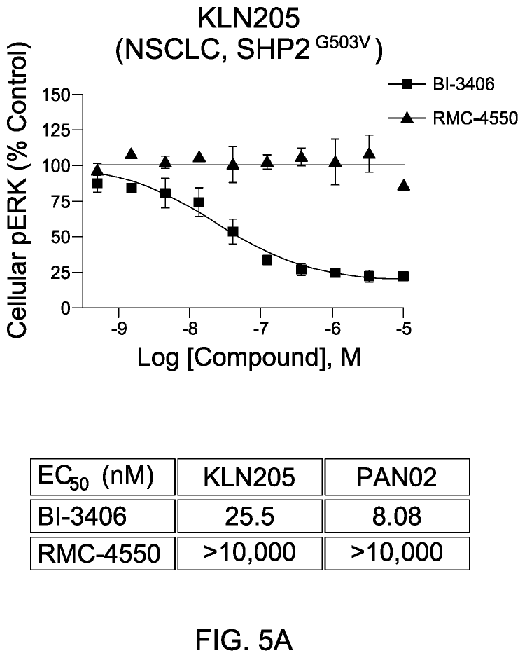

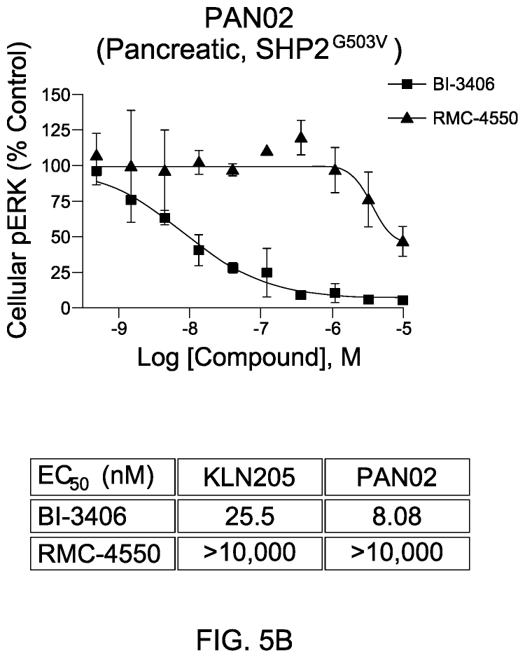

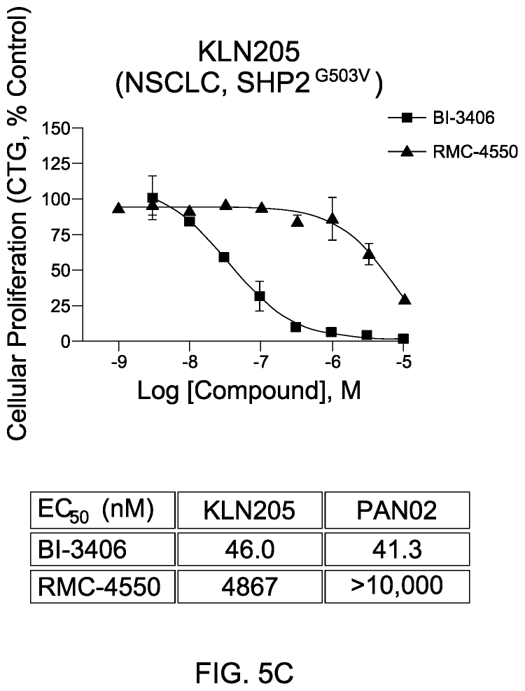

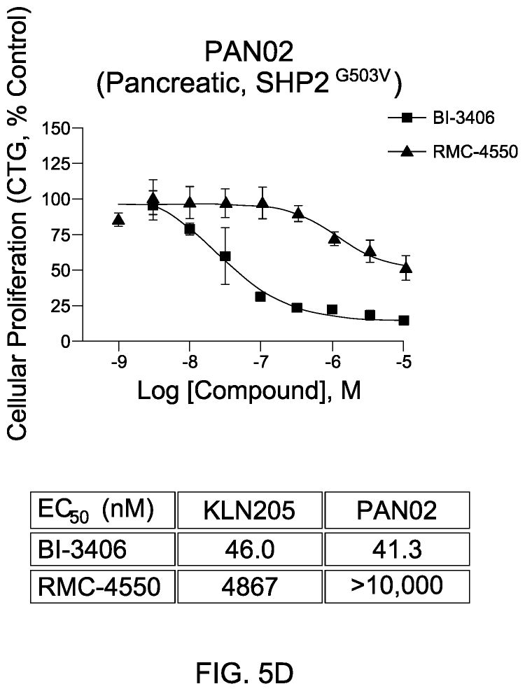

In this example, two mouse tumor cell lines, KLN205 (lung squamous cell carcinoma) and PAN02 (pancreatic cancer), were characterized. The mouse tumor cell lines contained the highly activating G503V SHP2 mutation. In cell culture experiments in vitro the SHP2 allosteric inhibitor RMC-4550 showed low or moderate inhibition of pERK and of cell growth even at the highest test concentration of 10 μM, whereas the SOS1 inhibitor BI-3406 potently inhibited pERK and growth in both cell lines. See FIGS. 5A through 5D.

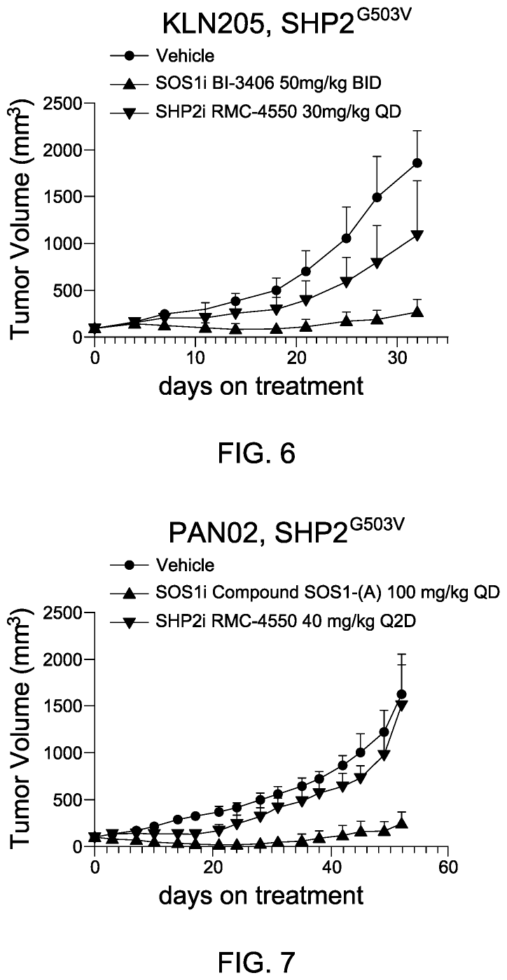

Consistent with in vitro observations, in both KLN205 tumor cell lines (See FIG. 6) and PAN02 tumor cell lines (See FIG. 7), BI-3406 treatment resulted in near complete inhibition of tumor growth in vivo, whereas the SHP2 allosteric inhibitor RMC-4550 resulted in less than 50% tumor growth inhibition.

The following methods were used to obtain the data shown in FIGS. 5A through 5D, 6, and 7.

### Determination of Sensitivity to SOS1 and SHP2 Inhibitors In Vivo

All studies were compliant with all relevant ethical regulations regarding animal research in accordance with approved institutional animal care and use committee IACUC protocols at HD Biosciences (San Diego, CA).

For KLN205 studies, female (6-8 weeks old) immunocompetent DBA/2 mice were implanted subcutaneously with 0.5E+06 KLN205 cells, Once tumors reached an average volume of 100 mm³ administration of RMC-4550 (30 mg/kg, by daily oral administration), BI-3406 (50 mg/kg, by twice daily oral administration), or vehicle (2% HPMC in 50 mM sodium citrate buffer) was initiated.

For PAN02 studies, female (6-8 weeks old) C57/B6 were implanted subcutaneously with 5E+06 PAN02 cells, Once tumors reached an average volume of 100 mm³ administration of RMC-4550 (40 mg/kg, by every 2 days oral administration), Compound SOS1-(A) (also called RMC-0331) (100 mg/kg, by daily oral administration), or vehicle (2% HPMC in 50 mM sodium citrate buffer) was initiated.

### Results

Activating mutation G503V induces resistance to allosteric SHP2 inhibition, but maintains sensitivity to SOS1 inhibitors in syngeneic mouse cell lines KLN205 and PAN02. See FIGS. 5A through 5D, which are graphs depicting the pERK AlphaLISA® assay. The associated tables with FIGS. 5A through 5D depict the results of the CellTiter-Glo® viability assay.

In KLN205 models containing SHP2 strongly activating mutant G503V, SOS1 inhibition (as exemplified by BI-3406) yielded substantially higher levels of tumor growth inhibition (TGI) than SHP2 inhibition (RMC-4550) immunocompetent mice. See FIG. 6.

In PAN02 models containing SHP2 strongly activating mutant G503V, SOS1 inhibition (as exemplified by Compound SOS1-(A) (also called RMC-0331)) yielded substantially higher levels of tumor growth inhibition (TGI) than SHP2 inhibition (RMC-4550) in immunocompetent mice (FIG. 7).

### Example 3. SHP2 Activating Mutations have Differential Effect on Cellular Dependence on SOS1 and SOS2

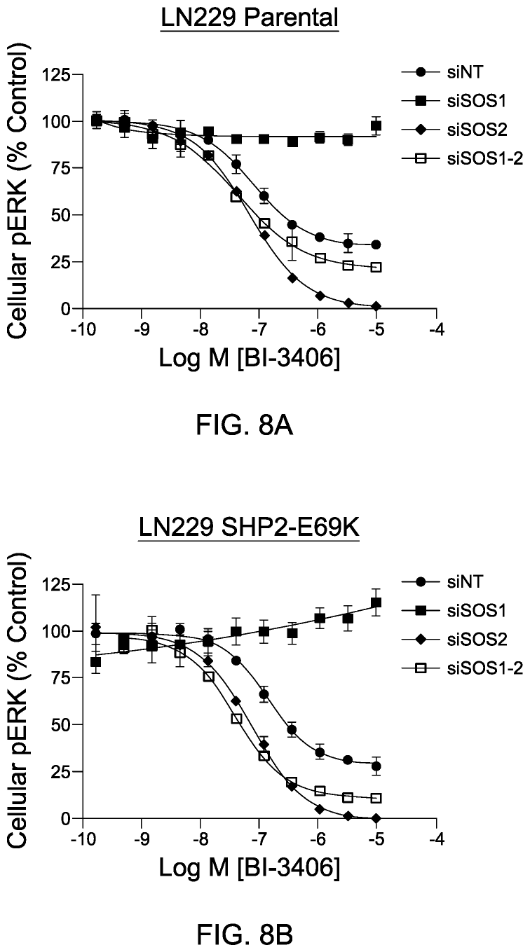

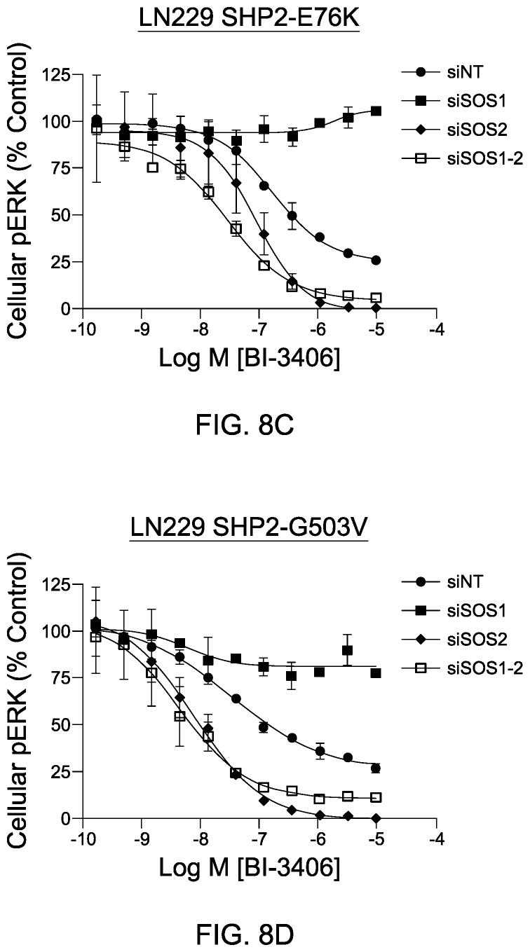

SHP2 activates MAPK pathway signaling in part by recruiting and activating SOS RAS guanine nucleotide exchange factors. There are two isoforms of SOS, SOS1 and SOS2. The precise roles of SOS1 and SOS2 remain to be elucidated, but SHP2 is assumed to activate both isoforms, whereas SOS1 inhibitors such as BI-3406 and Compound SOS1-(A) (also called RMC-0331) inhibit only SOS1. This is shown in LN229 cells with four different SHP2 mutations in FIGS. 8A through 8D. In all cases, genetic knockdown of SOS2 resulted in more complete depth of inhibition of pERK after exposure to BI-3406 compared with knockdown with a non-targeting control siRNA, consistent with SOS2 having an overlapping role with SOS1 in pathway activation and not being targeted by BI-3406. In all cases knockdown of SOS1 completely abrogated the effects of BI-3406, validating SOS1 as the target of BI-3406 and demonstrating SOS2 can compensate for SOS1 loss.

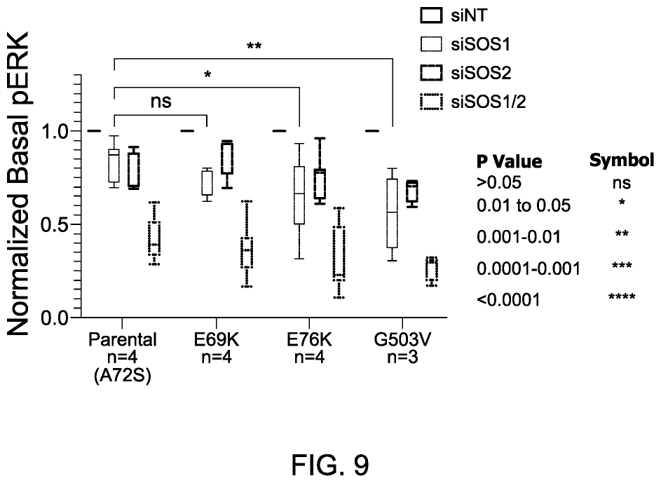

FIG. 9 shows the effect of genetic knockdown of SOS1, SOS2, or both on the basal pERK level in LN229 cells with different activating mutations in SHP2. LN229 cells with more strongly activating SHP2 mutations (E76K and G503V) exhibit greater sensitivity to knockdown of SOS1 and/or SOS2, as shown by the magnitude of decrease in basal pERK upon knockdown (FIG. 9). All cells are sensitive to simultaneous knockdown of SOS1 and SOS2, but unexpectedly, a trend is seen of greater effect of SOS1 knockdown in cells with more strongly activating SHP2 mutations. In contrast, SOS2 knockdown has only a small and similar effect in all cell lines. This suggests that strongly activating SHP2 mutations increase dependence on SOS1, and may therefore increase sensitivity to SOS1 inhibitors such as BI-3406 and Compound SOS1-(A) (also called RMC-0331).

In view of these results, inhibition of SOS1 in combination with SOS2 by a dual inhibitor is contemplated by the method of the present invention. Selective SOS1 inhibitors are also useful in view of the greater effect of SOS1 knockdown in cells with more strongly activating SHP2 mutations. The following methods were used to obtain the data shown in FIGS. 8A through 8D and 9.

### Generation of Isogenic LN229 SHP2 Mutant Cell Lines

An experimental system was created to test the activity of SHP2 mutants on an isogenic background. See FIGS. 4A through 4H, 8A through 8D, and 9. SHP2 mutations were introduced with Synthego CRISPR editing techniques, and LN229 variant cell lines stably expressing these mutations were generated.

### Determination of Sensitivity to SOS1 and SOS2 Knockdown

One day prior to transfection, LN229 cell lines with each indicated mutation were harvested and seeded in high glucose DMEM containing 2 mM L-glutamine, supplemented with 10% FBS in 96-well assay plates at a density of 10,000 cells/well. On the day of the transfection, transfection mixtures prepared according to Dharmafect I protocol. Per well, 1 μL of 5 μM siRNA in 10 μL OptiMEM, was combined with 0.2 μL Dharmafect I in 10 μL OptiMEM, plus 80 μL standard cell medium was added directly to cells after aspirating plating medium.

Per 100 wells in a single condition: 110 μL of 5 μM siRNA diluted with OptiMEM medium to 1.1 mL. Separately, 22 μL Dharmafect I diluted to 1.1 mL and each mixture incubated 5 minutes at room temperature. 1.1 mL siRNA mixture combined with 1.1 mL Dharmafect mixture, and incubated 20 minutes at room temperature. 2.2 mL siRNA/Dharmafect combined mixture diluted with standard cell culture medium (e.g. RPMI+10% FBS, no p/s) to 11 mL total. Cell medium aspirated for all wells to be transfected, 100 μL transfection mixture added to each well. Cells incubated with transfection mixture for 72 hours.

Cell medium aspirated, 50 μL standard cell culture medium added (10% DMEM, 10% FBS). 50 μL of 2× stock of compounds prepared from DMSO dilution series with 0.2% DMSO added to yield final DMSO concentration of 0.1% (i.e. 1:1000 final dilution of DMSO stock in standard medium). Plate returned to incubator for 3 hours.

After this incubation was complete, media was aspirated and cellular lysates prepared using lysis buffer provided with the AlphaLISA® detection kit (PerkinElmer). ERK1/2 phosphorylation at Thr202/Tyr204 was assayed using the AlphaLISA® SureFire® Ultra™ HV PERK Assay Kit (Perkin Elmer) following the manufacturer's instructions. Samples were read using an EnVision® Multilabel Plate Reader (Perkin Elmer) using standard AlphaLISA® settings. Assay data was plotted and EC50 values were determined using four-parameter concentration-response model in GraphPad Prism 7. Data provided are mean+/−standard deviation of duplicate values from representative experiments.

### Results

Cells were transfected with SOS1 or SOS2 siRNA, and incubated with BI-3406 prior to measurement of cellular pERK levels by AlphaLISA®. Cell lines confirmed to be sensitive to inhibition in non-targeting control with partial depth of inhibition, rescued from inhibition by BI-3406 by knockdown of SOS1, and sensitized by knockdown of SOS2 to restore full depth of inhibition. See FIGS. 8A through 8D.

Overall, all SHP2 activating mutants demonstrated significant reduction in basal pERK after SOS1 knockdown, and levels of basal pERK reduction correlate with biochemical potency for activated SHP2 variant. See FIG. 9.

### Example 4. Analysis of SHP2 Mutants

### Determining ΔGₒₚ and the Strength of Activating Mutations in SHP2

In the absence of activating signals, SHP2 adopts an auto-inhibited (“Closed”) conformation in which the N—SH2 domain binds over the active site, blocking access to substrates. Activation, by binding of the N- and C—SH2 domains to phosphotyrosine-containing sequences in effector proteins, causes the N—SH2 domain to move out of the active site, creating an active (“Open”) conformation. Allosteric inhibitors of SHP2 bind exclusively to the Closed conformation with affinity Kᵢ, and activating phosphopeptides (called “peptide” or “P” below) bind exclusively to the Open conformation with affinity K_{d}. The auto-inhibited conformation of SHP2 has an intrinsic stability described by the opening equilibrium constant Kₒₚ. All of these equilibrium constants can also be expressed as Gibbs free energies (ΔG), according to the equation:

$$Δ = - ( )$$

where R is the ideal gas constant (0.00198588 kcal/mol*K used in all analysis) and T is the absolute temperature (298 K used in all analysis).

The Kₒₚ or ΔGₒₚ governs the “activatability of SHP2, and is an intrinsic property of wild type SHP2, but can be changed by mutation. Some mutations decrease the ΔGₒₚ, making SHP2 both more sensitive to activation by phosphotyrosine-containing peptides, and also less sensitive to allosteric inhibitors. The magnitude of this change varies with mutation, and can be small (weakly-activating mutations), moderate (moderately activating mutations), or large (strongly activating mutations).

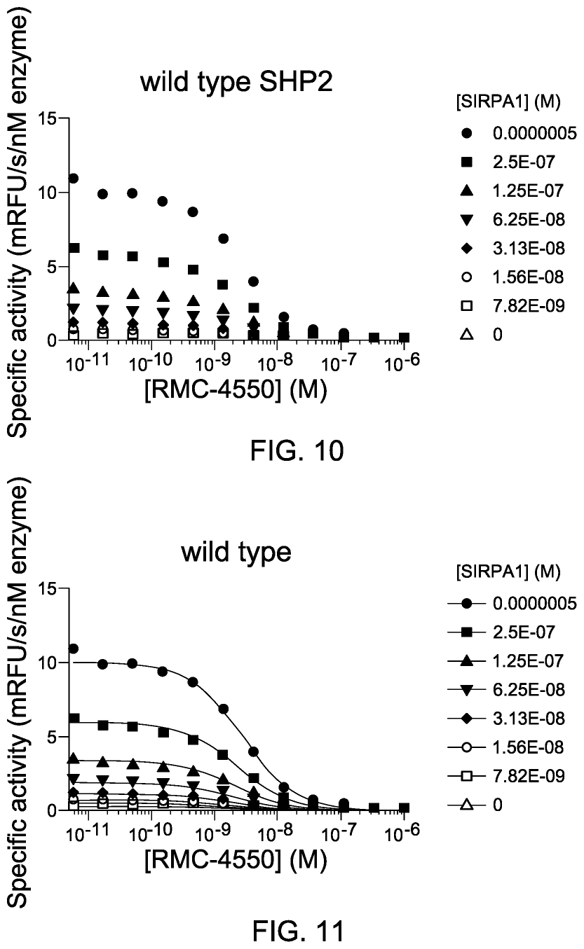

The ΔGₒₚ of wild type SHP2 or a mutant can be determined from a 2-dimensional concentration response experiment where the activity of SHP2 is measured as a function of varying concentrations of both an activating phosphopeptide (such as SIRPA¹) and an allosteric inhibitor (such as RMC-4550). The results of such an experiment are shown in FIG. 10.

In order to estimate ΔGₒₚ from this experiment, a model based on the following reaction scheme is fitted, which assumes affinity of inhibitor for open SHP2 and affinity of peptide for closed SHP2 are negligible.

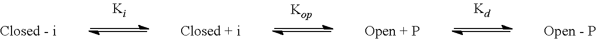

The following equilibrium constants can be defined:

$$= \frac{[ ]}{[ \text{?} ]} ( Stability of the closed conformation ) . \qquad 1$$

$$= \frac{[ ] [ ]}{[ \text{?} . ]} ( Dissociation constant for Peptide ( P ) and open conformation . \qquad 2$$

$$= \frac{[ ] [ ]}{[ \text{?} . ]} ( Dissociation constant for inhibitor ( i ) and closed conformation . \qquad 3$$

$$\text{?} \text{indicates text missing or illegible when filed}$$

The fraction of molecules in the open state (θₒₚ) is equal to

$$= \frac{[ ]}{[ \text{?} 2 ]} = \frac{[ ] + [ \text{?} . ]}{[ ] + [ ] + [ \text{?} . ] + [ \text{?} . ]}$$

$$\text{?} \text{indicates text missing or illegible when filed}$$

Dividing the numerator and denominator by [closed] gives

$$\frac{\frac{[ ]}{[ \text{?} ]} + \frac{[ \text{?} . ]}{[ ]}}{\frac{[ ]}{[ \text{?} ]} + \frac{[ ]}{[ \text{?} ]} + \frac{[ \text{?} . ]}{[ ]} + \frac{[ \text{?} . ]}{[ ]}}$$

$$\text{?} \text{indicates text missing or illegible when filed}$$

Note that [⋅]/[ ]=−[ ] and other fractions are equal to the equilibrium constants defined above. Substituting these values results in the following equation:

$$= \frac{\text{?} + \frac{}{} [ ]}{1 + \text{?} + \frac{}{} [ ] + \frac{[ ]}{}}$$

$$\text{?} \text{indicates text missing or illegible when filed}$$

Assuming that the activity of the closed conformation is negligible, the apparent activity is the product of the specific activity of the open conformation and the fraction open:

$$\text{?} = \text{?} . \text{?}$$

$$\text{?} \text{indicates text missing or illegible when filed}$$

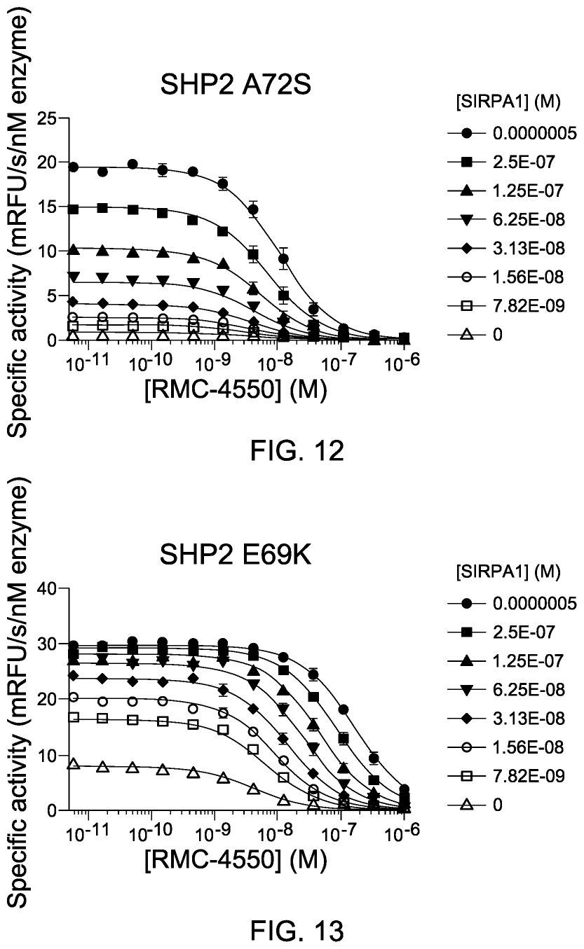

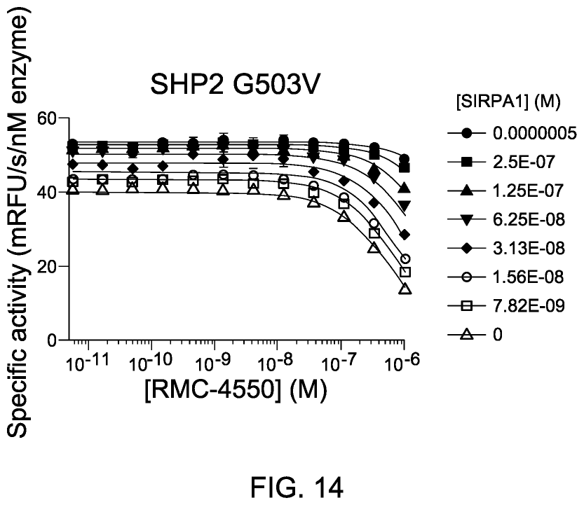

It is possible to fit this model to the experiment shown in FIG. 10 to estimate values of each parameter. Model fit curves are shown as lines in FIG. 11. The ΔGₒₚ from this fit is 2.74 kcal/mol. Examples are also shown of a weakly activating mutant (A⁷²S, 2.03 kcal/mol, FIG. 12), a moderately activating mutant (E69K, 0.61 kcal/mol, FIG. 13), and a strongly activating mutant (G503V, −0.62 kcal/mol, FIG. 14).

Model fit parameters for wild type SHP2 and 21 mutants are summarized in Table 1 above. ΔG_{peptide} corresponds to K_{d}, or affinity for activating phosphopeptide, and ΔGᵢ corresponds to Kᵢ, or affinity of RMC-4550 for the closed conformation of SHP2.

A weakly activating mutant is defined as one with a ΔGₒₚ not more than 1.5 kcal/mol below wild type SHP2. A moderately activating mutant has a ΔGₒₚ between 1.5 kcal/mol and 2.24 kcal/mol below wild-type. A strongly activating mutation has a ΔGₒₚ more than 2.24 kcal/mol below wild type.

### Methods

The effect of RMC-4550 and SIRPA, a peptide, on the hydrolysis of the fluorogenic small molecule substrate 6,8-Difluoro-4-Methylumbelliferyl Phosphate (DiFMUP) was determined. Each SHP2 variant was assayed in triplicate 96-well plates, at 8 different [SIRPA¹ peptide] and 12 different [RMC-4550].

SIRPA¹ (Peptide Sequence H₂N—

IT [Y]ADLNLP [PEG8]HTE [Y]ASIQTSK-NH₂ (ThermoFisher Custom Peptides), where brackets indicate phosphotyrosine) was prepared at a stock concentration of 10 μM (20× max final) in 50 mM HEPES pH 7.2, 0.02% BSA. Six serial 3-fold dilutions were prepared and one well was prepared with dilution buffer alone. RMC-4550 was prepared in 50 mM HEPES pH 7.2, 0.02% BSA at a concentration of 2 μM (20× final) and 10 serial 3-fold dilutions were prepared. One well was prepared with buffer alone. 20× dilution series of SIRPA¹ and RMC-4550 were mixed 1:1 in a matrix fashion in a 96-well plate to prepare a 10× peptide+compound plate, with decreasing [RMC-4550] in rows and decreasing [SIRPA¹] in columns. Enzyme was prepared at 2× final concentration in 100 mM HEPES pH 7.2, 200 mM NaCl, 1 mM EDTA, 2 mM DTT, 0.002% Brij35. All mutants were assayed at a final enzyme concentration of 0.5 nM and wild-type was assayed at 1 nM.

50 μl 2× enzyme stock was added to 96 well black polystyrene plates using an Agilent Bravo and mixed with 10 μl compound/peptide stock. Triplicate plates were prepared for each enzyme. Plates were incubated 20-40 minutes after preparation, and then 40 μl 50 μM DiFMUP in water was added to each well using a MultiDrop® Combi. Plates were shaken and read in a Spectramax® M5 plate reader in kinetic mode for five minutes, with excitation at 358 nm and emission at 450 nm. Linear signal vs. time curves were fit in SoftMax Pro and slopes were exported to Excel. Slopes from Softmax were converted to specific activity by dividing by the final enzyme concentration.

### Example 5. SOS1 Inhibitors Shows Single Agent Anti-Tumor Activity In Vivo in Tumor Models Bearing SHP2 Activating Mutations

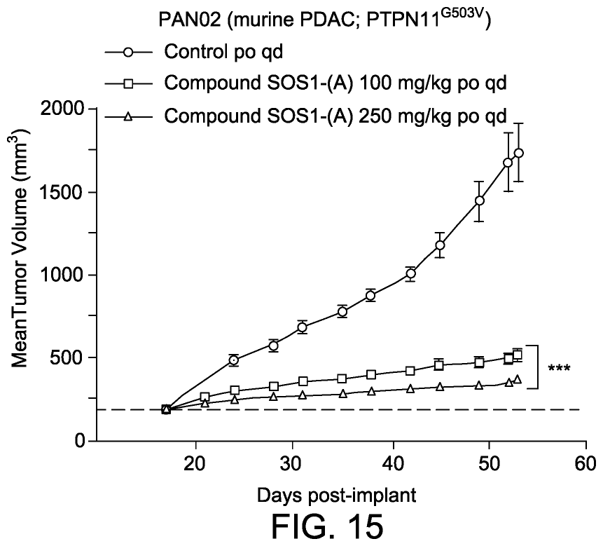

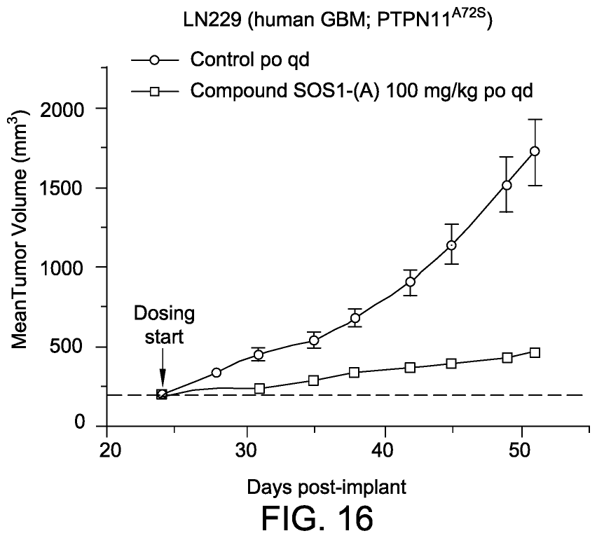

In this example, two mouse tumor cell lines, PAN02 (pancreatic cancer, immunocompetent) and LN229 CDX (glioma, immunocompromised), were characterized. The PAN02 mouse tumor cell line contained the highly activating G503V SHP2 mutation. The LN229 CDX contained the highly activating A⁷²S SHP2 mutation. In both PAN02 tumor cell lines (See FIG. 15; 100 mg/kg po qd and 250 mg/kg po qd) and LN229 CDX tumor cell lines (See FIG. 16; 100 mg/kg po qd), Compound SOS1-(A) (also called RMC-0331) treatment resulted in near complete inhibition of tumor growth in vivo.

The effect of the SOS1 inhibitor RMC-0331 on tumor cell growth in vivo was evaluated in (A) PAN02 Syngeneic model using female C57/BL6 mice and (B) LN229 xenograft model using female balb/c athymic nude mice (6-8 weeks old). Mice were implanted with (A) PAN02 tumor cells (1e6 cells/mouse) or (B) LN229 tumor cells in 50% matrigel (10e6 cells/mouse) subcutaneously in the flank. Once tumors reached an average size of ˜200 mm³ mice were randomized to treatment groups and administration of test article or vehicle (2% HPMC E-50, 0.5% Tween-80 in 50 mM Sodium Citrate Buffer, pH 4.0). Body weight and tumor volume (using digital calipers) were measured twice a week until study endpoints. Compounds were administered by oral gavage daily.

### Example 6. SOS1 Inhibitor Shows Combination Benefits with RAS(ON) Inhibitors In Vitro

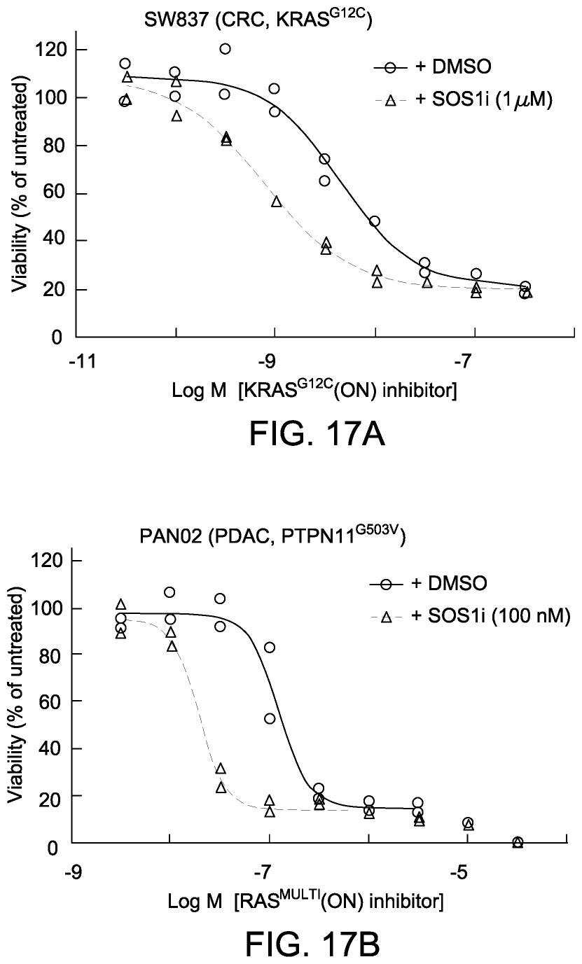

In this example, two cell lines, SW837 (colorectal cancer, human) and PAN02 (pancreatic cancer, mouse), were characterized. The SW837 cell line contained the KRAS^{G12C} mutation. The PAN02 mouse tumor cell line contained the highly activating G503V SHP2 mutation. The cell lines were treated with DMSO (vehicle) and a constant concentration of SOS1 inhibitor, Compound SOS1-(B). The cell lines were treated with varying concentration of a RAS inhibitor, Compound RAS-(E). FIGS. 17A (1 μM of SOS1 inhibitor; SW837 cell line; variable concentration or RAS inhibitor) and 17B (100 nM of SOS1 inhibitor; PAN02 cell line; variable concentration or RAS inhibitor) illustrate the viability of the cells as a function of RAS inhibitor, which depict the additive effect of SOS1 and RAS inhibition on cell viability. The data were obtained according to the 2-dimensional potency shifts experimental protocol.

### Example 7. SOS1 Inhibitor Shows Combination Benefits with RAS(ON) Inhibitors In Vivo

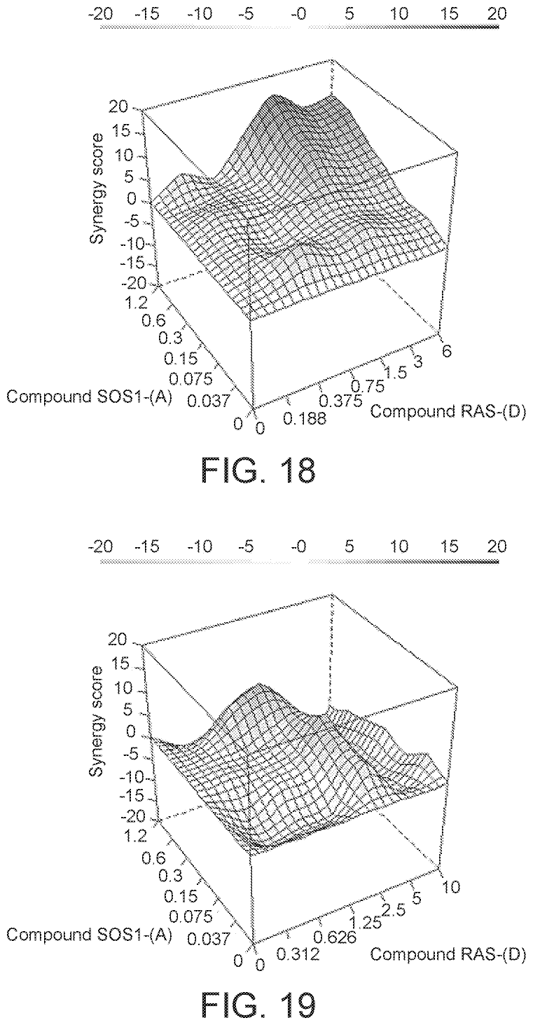

In this example, two mouse tumor cell lines, KLN205 (lung squamous cell carcinoma) and PAN02 (pancreatic cancer), were characterized. The mouse tumor cell lines contained the highly activating G503V SHP2 mutation. FIG. 18 is a Loewe 3D response surface plot showing the in vitro combination effect of SOS1 inhibitor Compound SOS1-(A) (also called RMC-0331) and RAS^{MULTI} (ON) inhibitor Compound RAS-(D) observed in Pan02 cells. A synergy score >5 at any point on the plot indicates a positive interaction between the two compounds. FIG. 19 is a Loewe 3D response surface plot showing the in vitro combination effect of SOS1 inhibitor Compound SOS1-(A) (also called RMC-0331) and RAS^{MULTI} (ON) inhibitor Compound RAS-(D) observed in KLN205 cells. A synergy score >5 at any point on the plot indicates a positive interaction between the two compounds. These data were obtained according to the CrownSyn™ method.

### Example 8. SOS1 Inhibitors Shows Single Agent Anti-Tumor Activity In Vivo in Tumor Models Bearing SHP2 Activating Mutations

### Methods:

The anti-tumor efficacy of Compound SOS1-(C) was evaluated in comparison to RMC-4550 and Cobimetinib, in a human engineered model of PTPN11-mutant glioblastoma, LN229.E76K. 6-7 week athymic nude mice were implanted with LN229.E76K in 50% matirgel (10×10⁶ cells/mouse) subcutaneously in the flank. Once tumors reached an average size of 200 mm³, mice were randomized into treatment groups to start the administration of test articles or control. All treatments were administered daily by oral gavage. Body weight and tumor volume (using calipers) was measured twice weekly until study endpoint.

### Results:

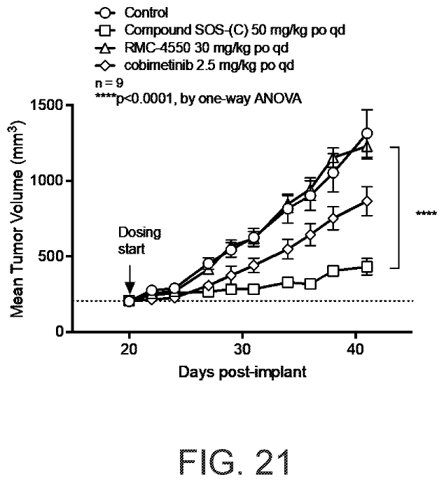

As shown in FIG. 21, single-agent Compound SOS1-(C) administered at 50 mg/kg PO daily led to a tumor growth inhibition (TGI) of 80%, single-agent RMC-4550 administered at 30 mg/kg PO daily led to a TGI of 7%, and cobimetinib administered at 2.5 mg/kg PO daily led to TGI of 41% in the LN229.E76K GMB CDX model with an engineered PTPN11E76K mutation. The anti-tumor activity of Compound SOS1-(C) and cobimetinib were statistically significant from the vehicle control group (**** p<0.0001 and *p<0.05 respectively), while response to RMC-4550 treatment was not statistically significant, assessed by an ordinary one-way ANOVA of tumor volumes along with multiple comparisons via a post-hoc Tukey's test in GraphPad Prism software. All treatment arms were well tolerated.

### Example 9. SOS1 Inhibitor Shows Combination Benefits In Vivo in Tumor Models Bearing SHP2 Activating Mutations

### Methods:

The combinatorial effects of Compound SOS1-(C) with RAS-(E) on tumor cell growth in vivo were evaluated in the murine pancreatic ductal adenocarcinoma PAN02 PTPN11G503V syngeneic model using female C57BL/6J mice (6-7 weeks old). Mice were implanted with PAN02 tumor cells in PBS (5 ×10⁶cells/mouse) subcutaneously in the flank. Once tumors reached an average size of ˜130 mm³, mice were randomized to treatment groups to start the administration of test articles or control. All test articles were administered by oral gavage daily. Body weight and tumor volume (using calipers) was measured twice weekly until study endpoints.

### Results:

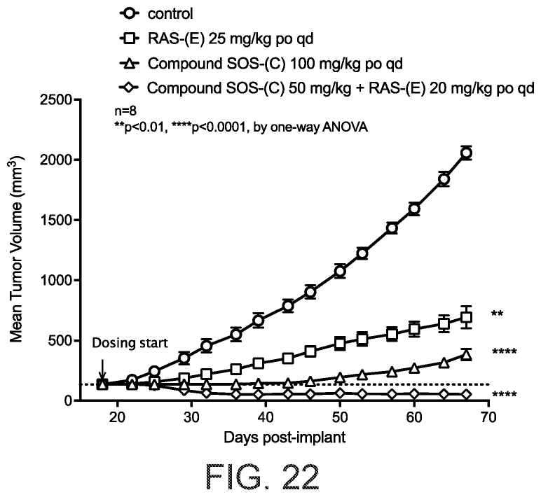

As shown in FIG. 22, single-agent Compound SOS1-(C) administered at 50 mg/kg PO daily led to a tumor growth inhibition (TGI) of 94%, single-agent RAS-(E) administered at 25 mg/kg PO daily led to a TGI of 64%, in the PAN02 murine PDAC CDX model with a PTPN11^{G503V} mutation. However, the anti-tumor activity by the combination treatment led to tumor volume regressions of 54%. All treatment arms were statistically significant from the vehicle control group (** p<0.01, and **** p<0.0001, assessed by an ordinary one-way ANOVA of tumor volumes along with multiple comparisons via a post-hoc Tukey's test in GraphPad Prism software).

### Example 10. SOS1 Inhibitor Shows Combination Benefits In Vivo in Tumor Models Bearing SHP2 Activating Mutations

### Methods:

The combinatorial effects of Compound SOS1-(C) with cobimetinib on tumor cell growth in vivo were evaluated in the murine pancreatic ductal adenocarcinoma PAN02 PTPN11G503V syngeneic model using female C57BL/6J mice (6-7 weeks old). Mice were implanted with PAN02 tumor cells in PBS (5 ×10⁶cells/mouse) subcutaneously in the flank. Once tumors reached an average size of ˜130 mm³, mice were randomized to treatment groups to start the administration of test articles or control. All test articles were administered by oral gavage daily. Body weight and tumor volume (using calipers) was measured twice weekly until study endpoints.

### Results:

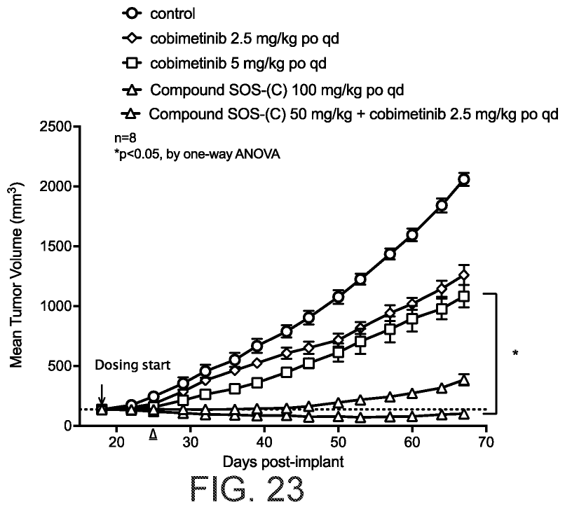

As shown in FIG. 23, single-agent Compound SOS1-(C) administered at 100 mg/kg PO daily led to a tumor growth inhibition (TGI) of 94%, single-agent cobimetinib administered at 2.5 or 5 mg/kg PO daily led to a TGI of 38% and 49% respectively, in the PAN02 murine PDAC CDX model with a PTPN11G503V mutation. However, the anti-tumor activity by the combination treatment led to 42% tumor volume regressions and was statistically significant from the vehicle control group (**** p<0.0001) and from the 5 mg/kg cobimetinib single agent group (*p<0.05), assessed by an ordinary one-way ANOVA of tumor volumes along with multiple comparisons via a post-hoc Tukey's test in GraphPad Prism software. All treatment arms were well tolerated.

### Example 11. SOS1 Inhibitor Shows Combination Benefits In Vivo in Tumor Models Bearing SHP2 Activating Mutations

### Methods:

The combinatorial effects of Compound SOS1-(B) with cobimetinib on tumor cell growth in vivo were evaluated in a human engineered model of PTPN11-mutant glioblastoma, LN229.E76K xenograft model using female athymic nude mice (6-7 weeks old). Mice were implanted with LN229.E76K tumor cells in 50% Matrigel (10×10⁶cells/mouse) subcutaneously in the flank. Once tumors reached an average size of ˜200 mm3, mice were randomized to treatment groups to start the administration of test articles or control. All test articles were administered by oral gavage daily. Body weight and tumor volume (using calipers) was measured twice weekly until study endpoints.

### Results:

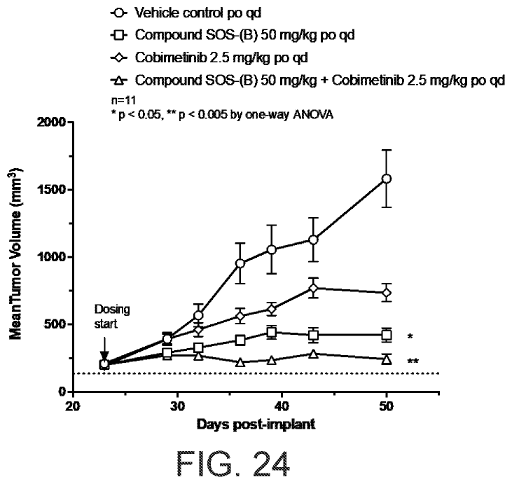

As shown in FIG. 24, single-agent Compound SOS1-(B) administered at 50 mg/kg PO daily led to a tumor growth inhibition (TGI) of 85%, single-agent cobimetinib administered at 2.5 PO daily led to a TGI of 62% in the LN229.E76K GMB CDX model with an engineered PTPN11E76K mutation. The anti-tumor activity by the combination treatment led to TGI of 98% and was statistically significant from the vehicle control group (** p<0.005) assessed by an ordinary one-way ANOVA of tumor volumes along with multiple comparisons via a post-hoc Tukey's test in GraphPad Prism software. All treatment arms were well tolerated. Single agent treatment with cobimetinib at 2.5 mg/kg was not statistically different from vehicle treatment. All treatment arms were well tolerated.

### Example 12. SOS1 Inhibitor Shows Combination Benefits In Vivo in Tumor Models Bearing SHP2 Activating Mutations

### Methods:

The combinatorial effects of Compound SOS1-(C) with Abemaciclib on tumor cell growth in vivo were evaluated in the murine pancreatic ductal adenocarcinoma PAN02 PTPN11G503V syngeneic model using female C57BL/6J mice (6-7 weeks old). Mice were implanted with PAN02 tumor cells in PBS (5 ×10⁶cells/mouse) subcutaneously in the flank. Once tumors reached an average size of ˜130 mm3, mice were randomized to treatment groups to start the administration of test articles or control. All test articles were administered by oral gavage daily. Body weight and tumor volume (using calipers) was measured twice weekly until study endpoints.

### Results:

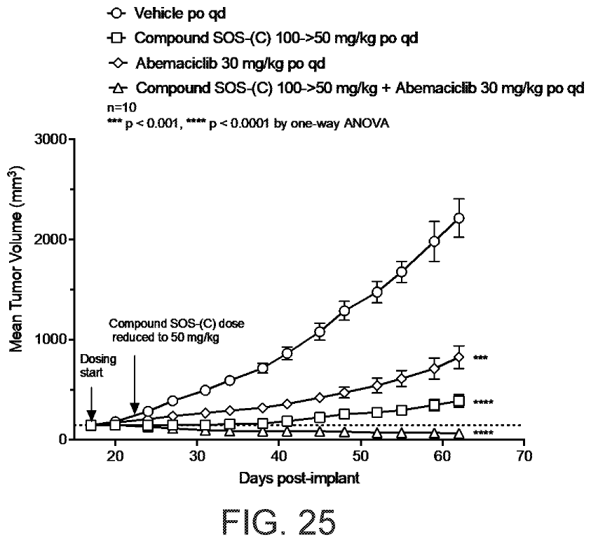

As shown in FIG. 25, single-agent Compound SOS1-(C) led to tumor growth inhibition (TGI) of 78%, single-agent abemaciclib administered at 30 mg/kg PO daily led to a TGI of 52%, in the PAN02 murine PDAC CDX model with a PTPN11G503V mutation. Both single agent treatments were statistically significant from the vehicle control group (**** p<0.0001, and *** p<0.001 respectively). However, the anti-tumor activity by the combination treatment led to 97% tumor volume regressions and was statistically significant from the vehicle control group (**** p<0.0001) assessed by an ordinary one-way ANOVA of tumor volumes along with multiple comparisons via a post-hoc Tukey's test in GraphPad Prism software. All treatment arms were well tolerated. Compound SOS1-(C) was initially administered at 100 mg/kg PO daily for 7 days and then reduced to 50 mg/kg PO daily for the remainder of the study in both the single agent and combination treatment arms.

### Example 13. SOS1 Inhibitor Shows Combination Benefits In Vivo in Tumor Models Bearing SHP2 Activating Mutations

### Methods:

The combination effects of SOS1-(C) with anti-PD1 on tumor cell growth in vivo were evaluated in the mouse syngeneic pancreatic ductal adenocarcinoma line PAN02 carrying a SHP2G503V mutation using female C57BL/6 mice (6-8 weeks old). Mice were implanted with PAN02 tumors (5×10⁶ cells/mouse) subcutaneously in the flank. Once tumors reached an average size of ˜105 mm3, mice were randomized to treatment groups to start the administration of test articles or vehicle. Anti-PD1 (cloneRMP1-4) was administered by intraperitoneal injection twice weekly, and SOS1-(C) was administered by oral gavage daily. Body weight and tumor volume (using calipers) was measured twice weekly until study endpoints.

### Results:

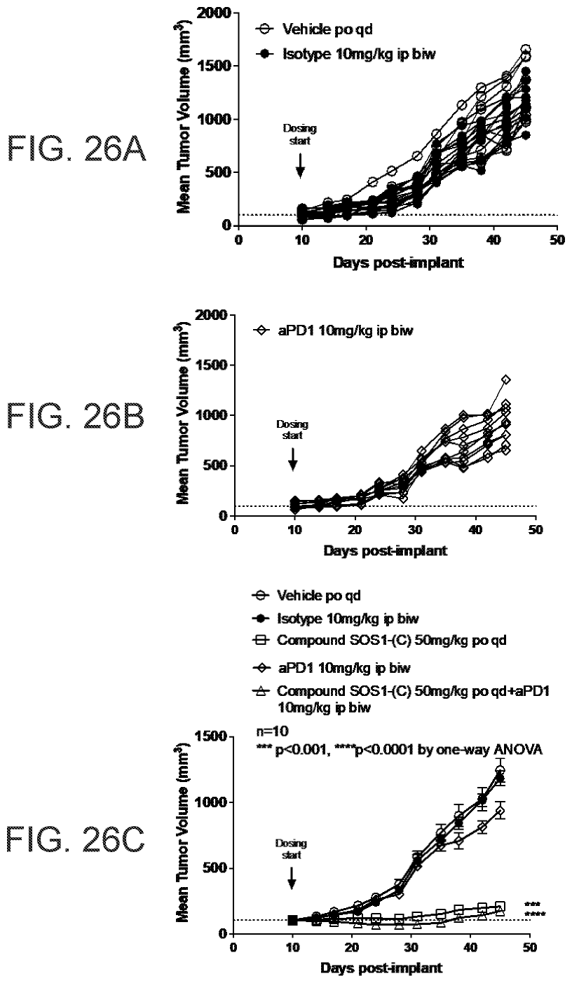

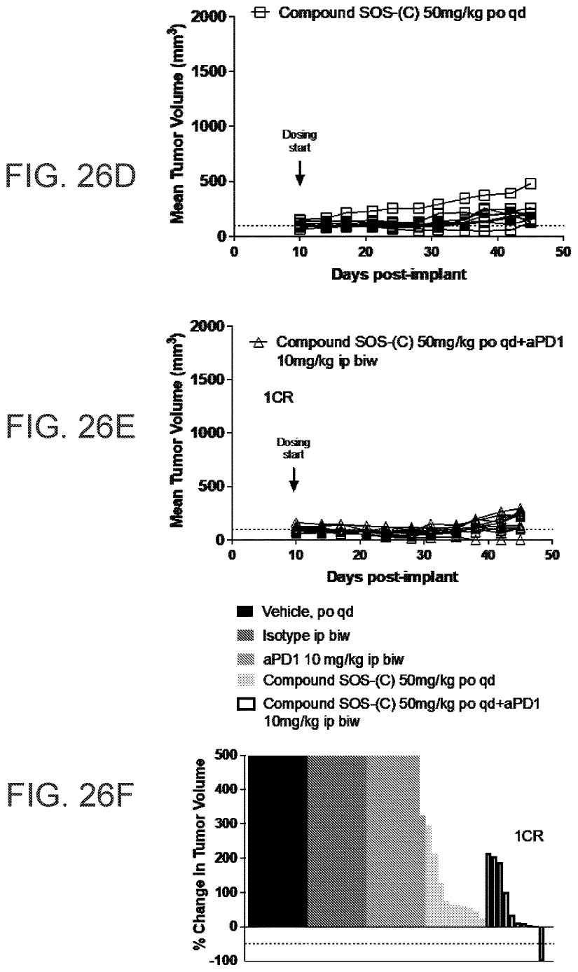

As shown in FIGS. 26A, 26B, 26C, 26D, 26E, and 26F, single-agent SOS1-(C) administered at 50 mg/kg PO daily led to a tumor growth inhibition (TGI) of 90.91%, and single-agent anti-PD1 administered at 10 mg/kg IP twice weekly led to a TGI of 26.75% in the PAN02 syngeneic mouse PDAC model with a SHP2G503V mutation. The combination led to a TGI of 93.96% and a complete tumor regression in one mouse in the PAN02 model. The anti-tumor activity of SOS1-(C) monotherapy was statistically significant with *** p<0.001 and the anti-tumor activity by the combination treatment was statistically significant from the vehicle control group, with **** p<0.0001, assessed by an ordinary One-way ANOVA of tumor volumes along with multiple comparisons via a post-hoc Tukey's test in GraphPad Prism software. Spaghetti plots show individual tumor responses. Waterfall plot shows individual tumor responses at the end of study, 1/10 tumors from the combination group showed complete regression. The combination treatment was well tolerated.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### APPENDIX A

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### SUMMARY

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

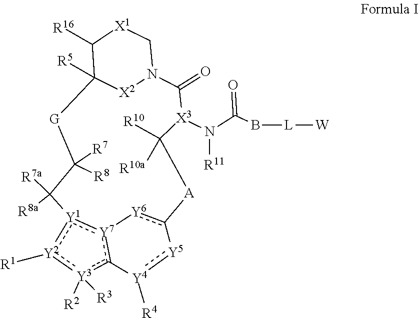

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene; G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; R¹⁰ is hydrogen, halo, hydroxy, C₁-C₃ alkoxy, or C₁-C₃ alkyl; R^{10a} is hydrogen or halo;
R¹¹ is hydrogen or C₁-C₃ alkyl. Also provided are pharmaceutical compositions comprising a compound of Formula I, or a pharmaceutically acceptable salt thereof, and a pharmaceutically acceptable excipient; and
R¹⁶ is hydrogen or C₁-C₃ alkyl (e.g., methyl).

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1A: A compound of the present invention, Compound A, exhibits PK-dependent RAS pathway modulation in a Capan-2 CDX model (PDAC, KRAS G12V/WT). Single dose compared to twice administered PK/PD measurement of Compound A. Second dose of Compound A delivered 8 hours following first dose, depicted by black arrow. All dose levels well tolerated. Tumor DUSP6 mRNA expression as percent of control graphed as bars on left y-axis. Dotted line indicates return to control level of DUSP6. Unbound plasma PK (nM) graphed as lines, plotted in Log 10 scale on right y-axis. N=3/time point. Error bars represent standard error of the mean.

FIG. 1B: Combinatorial anti-tumor activity with a compound of the present invention, Compound A, and upstream SHP2 inhibition in a Capan-2 CDX model (PDAC, KRAS G12V/WT). Capan-2 cells were implanted in 50% Matrigel. Animals were randomized and treatment was initiated at average tumor volume of ˜180 mm3. Animals were dosed with SHP2 inhibitor RMC-4550 20 mg/kg po q2d, Compound A 100 mg/kg po bid, combination RMC-4550 and Compound A, or Control for 40 days. All dose levels were tolerated. n=10/group (n=9 in Combination arm). Ns=no significance; *** p<0.001 by one-way ANOVA.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium;
halogen; —(CH₂)₀₋₄R^{∘}; —(CH₂)₀₋₄OR^{∘}; —O (CH₂)₀₋₄R^{∘};
—O—(CH₂)₀₋₄C(O)OR^{∘}; —(CH₂)₀₋₄CH (OR^{∘})₂; —(CH₂)₀₋₄SR^{∘}; —(CH₂)₀₋₄Ph, which may be substituted with R^{∘}; —(CH₂)₀₋₄O (CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or
cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S) R^{∘};)
(CH₂)₀₋₄N(R^{∘}C(O)NR^{∘}₂; —N(R^{∘}C(S)NR^{∘}₂; —(CH₂)₀₋₄N(R^{∘}C(O)OR^{∘}; —N(R^{∘}N(R^{∘}C(O)R^{∘}; —N(R^{∘}N(R^{∘}) C(O)NR^{∘}₂; —N(R^{∘}N(R^{∘}C(O)OR^{∘}; —(CH₂)₀₋₄C(O)R^{∘}; —C(S) R^{∘}; —(CH₂)₀₋₄C(O)OR^{∘}; —(CH₂)₀₋₄—C(O)—N(R^{∘})₂; —(CH₂)₀₋₄—C(O)—N(R^{∘}—S(O)₂—R^{∘}; —C(NCN) NR^{∘}₂; —(CH₂)₀₋₄C(O) SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S) SR^{∘}; —(CH₂)₀₋₄SC(O)R^{∘}; —(CH₂)₀₋₄C(O)NR^{∘}₂; —C(S) NR^{∘}₂; —C(S) SR^{∘}; —(CH₂)₀₋₄OC(O)NR^{∘}₂; —C(O)N (OR^{∘}) R^{∘}; —C(O) C(O)R^{∘}; —C(O) CH₂C(O)R^{∘}; —C(NOR^{∘}) R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N (R^{∘})S(O)₂NR^{∘}₂; —N(R^{∘})S(O)₂R^{∘}; —N(OR^{∘}) R^{∘}; —C(NOR^{∘}) NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘}) R^{∘}, —SiR^{∘}₃; —(C₁₋₄ straight or branched) alkylene)O—N(R^{∘}₂; or —(C₁₋₄ straight or branched) alkylene) C(O)O—N(R^{∘}₂, wherein each R^{∘} may be substituted as defined below and is independently hydrogen, —C₁₋₆ aliphatic, —CH₂Ph, —O (CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•},
(haloR^{•}), —(CH₂)₀₋₂OH, —(CH₂)₀₋₂OR*, —(CH₂)₀₋₂CH (OR^{•})₂; —O (haloR^{•}), —CN, —N₃, —(CH₂)₀₋₂C(O)R^{•}, —(CH₂)₀₋₂C(O)OH, (CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{•}, —(C₁₋₄ straight or branched alkylene) C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include:—O (CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include
halogen, —R^{•}-(haloR^{•}), —OH, —OR^{•},
—O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O) C(O)R^{†}, —C(O) CH₂C(O)R^{†}, —S(O)₂R^{†}, —S(O)₂NR^{†}₂, —C(S) NR^{†}₂, —C(N H) NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}-(haloR^{•}), —OH, —OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

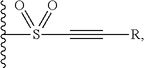

wherein R is any chemically feasible substituent described herein.

The term “amino,” as used herein, represents-N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term-N(C(O)—(C₀-C₅ alkylene-H)-includes-N(C(O)—(Co alkylene-H)—, which is also represented by-N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O) (OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

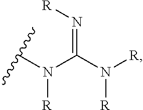

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more-OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC₅₀ of 2 μM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAFRBD construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras: RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

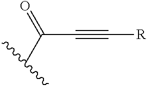

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) may contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Accordingly, provided herein is a compound, or pharmaceutically acceptable salt thereof, having the structure of Formula 00:

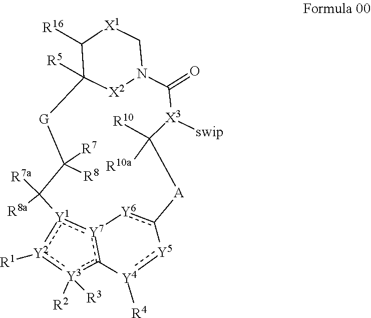

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
swIp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl (e.g., methyl). In some embodiments, the resulting compound is capable of achieving an IC₅₀ of 2 μM or less (e.g., 1.5 μM, 1 μM, 500 nM, or 100 nM or less) in the Ras-RAF disruption assay protocol described herein.

Accordingly, provided herein is a compound, or pharmaceutically acceptable salt thereof, having the structure of Formula I:

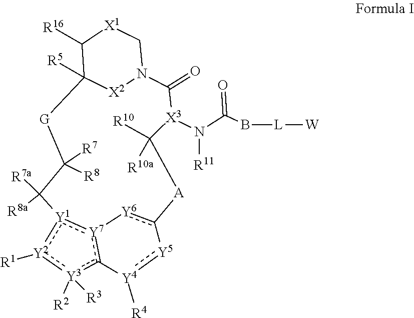

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl; R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the disclosure features a compound, or pharmaceutically acceptable salt thereof, of structural Formula Ia:

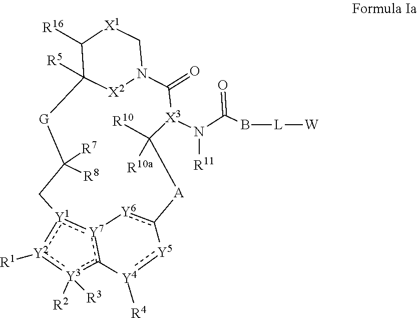

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula Ib:

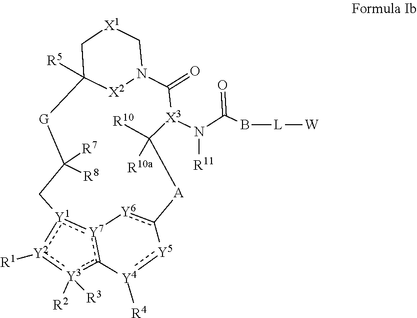

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, G is optionally substituted C₁-C₄ heteroalkylene.

In some embodiments, a compound of the present invention has the structure of Formula Ic, or a pharmaceutically acceptable salt thereof:

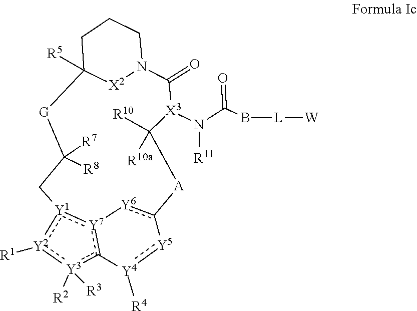

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, X² is NH. In some embodiments, X³ is CH.

In some embodiments of compounds of the present invention, R¹¹ is hydrogen. In some embodiments, R¹¹ is C₁-C₃ alkyl. In some embodiments, R¹¹ is methyl.

In some embodiments, a compound of the present invention has the structure of Formula Id, or a pharmaceutically acceptable salt thereof:

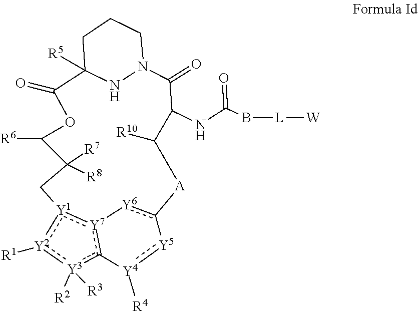

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH (Br)—. In some embodiments, X¹ is —CH (CH₃)—.

In some embodiments of compounds of the present invention, R³ is absent.

In some embodiments of compounds of the present invention, R⁴ is hydrogen.

In some embodiments of compounds of the present invention, R⁵ is hydrogen. In some embodiments, R⁵ is C₁-C₄ alkyl optionally substituted with halogen. In some embodiments, R⁵ is methyl.

In some embodiments of compounds of the present invention, Y⁴ is C. In some embodiments, Y⁵ is CH. In some embodiments, Y⁶ is CH. In some embodiments, Y¹ is C. In some embodiments, Y² is C. In some embodiments, Y³ is N. In some embodiments, Y⁷ is C.

In some embodiments, a compound of the present invention has the structure of Formula Ie, or a pharmaceutically acceptable salt thereof:

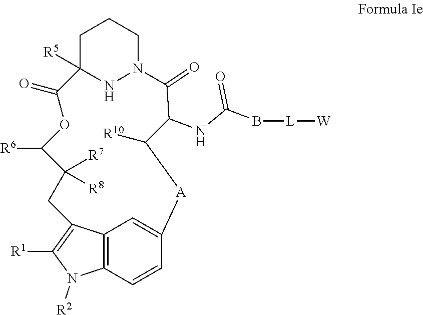

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, R⁶ is hydrogen.

In some embodiments of compounds of the present invention, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl. In some embodiments, R² is fluoro C₁-C₆ alkyl, such as-CH₂CH₂F, —CH₂CHF₂, or —CH₂CF₃.

In some embodiments of compounds of the present invention, R⁷ is optionally substituted C₁-C₃ alkyl. In some embodiments, R⁷ is C₁-C₃ alkyl.

In some embodiments of compounds of the present invention, R⁸ is optionally substituted C₁-C₃ alkyl. In some embodiments, R⁸ is C₁-C₃ alkyl, such as methyl.

In some embodiments, a compound of the present invention has the structure of Formula If, or a pharmaceutically acceptable salt thereof:

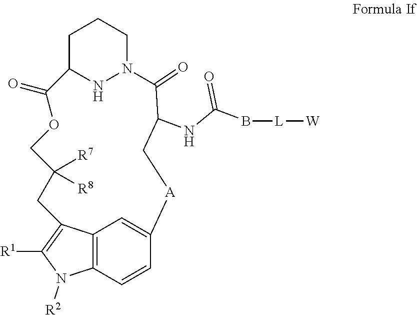

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, R¹ is 5 to 10-membered heteroaryl. In some embodiments, R¹ is optionally substituted 6-membered aryl or optionally substituted 6-membered heteroaryl.

In some embodiments of compounds of the present invention, R¹ is

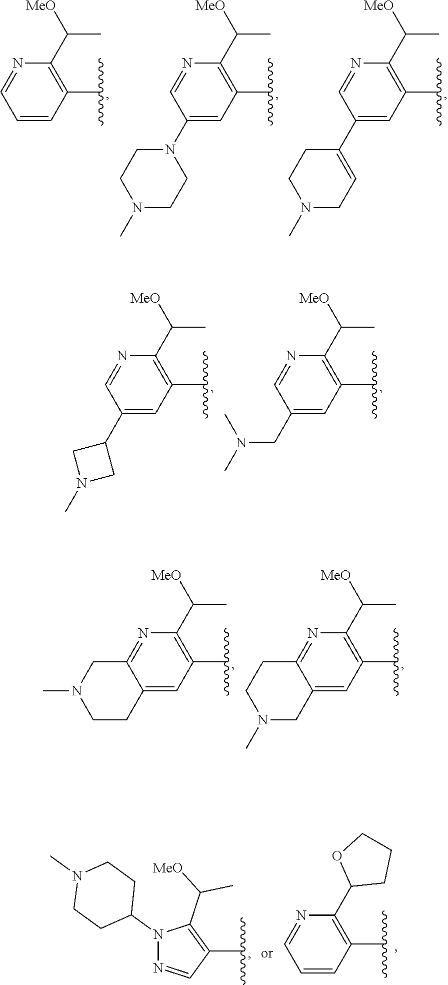

or a stereoisomer thereof. In some embodiments, R¹ is

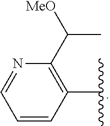 [SMILES: *c1cccnc1C(C)OC]

or a stereoisomer thereof. In some embodiments, R¹ is

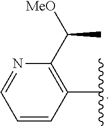 [SMILES: *c1cccnc1[C@H](C)OC]

In some embodiments, R¹ is

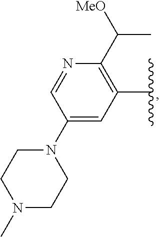 [SMILES: *c1cc(N2CCN(C)CC2)cnc1C(C)OC]

or a stereoisomer thereof. In some embodiments, R¹ is

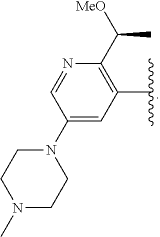 [SMILES: *c1cc(N2CCN(C)CC2)cnc1[C@H](C)OC]

In some embodiments, a compound of the present invention has the structure of Formula Ig, or a pharmaceutically acceptable salt thereof:

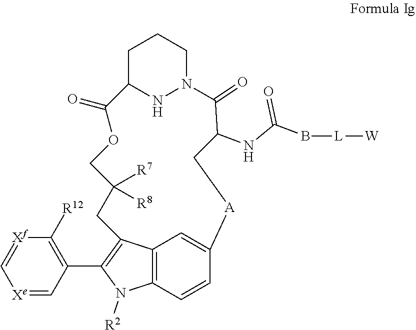

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of compounds of the present invention, X^{e} is N and X^{f} is CH. In some embodiments, X^{e} is CH and X^{f} is N. In some embodiments, X^{e} is CR¹⁷ and X^{f} is N.

In some embodiments of compounds of the present invention, R¹² is optionally substituted C₁-C₆ heteroalkyl. In some embodiments, R¹² is

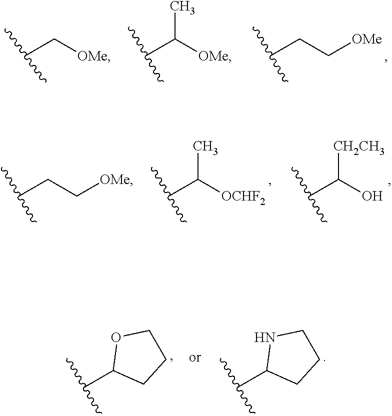

In some embodiments, a compound of the present invention has the structure of Formula Ih, or a pharmaceutically acceptable salt thereof:

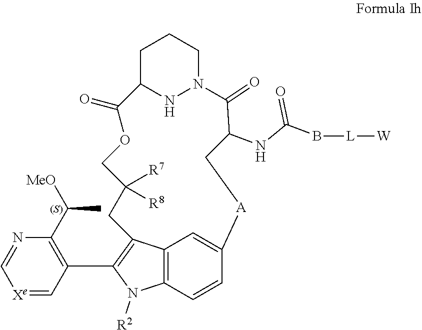

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, a compound of the present invention has the structure of Formula Ii, or a pharmaceutically acceptable salt thereof:

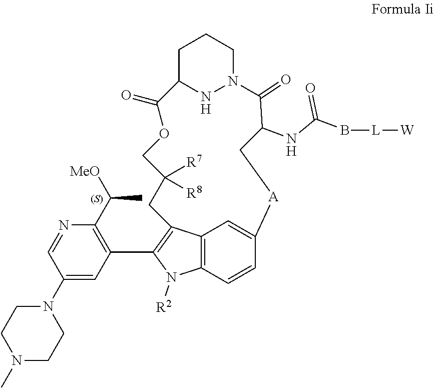

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, A is optionally substituted 6-membered arylene. In some embodiments, A has the structure:

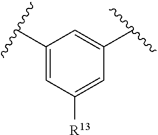

wherein R¹³ is hydrogen, hydroxy, amino, cyano, optionally substituted C₁-C₆ alkyl, or optionally substituted C₁-C₆ heteroalkyl. In some embodiments, R¹³ is hydrogen. In some embodiments, R¹³ is hydroxy. In some embodiments, A is an optionally substituted 5 to 10-membered heteroarylene. In some embodiments, A is:

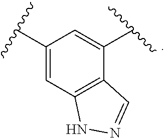 [SMILES: *c1cc(*)c2cn[nH]c2c1]

In some embodiments, A is optionally substituted 5 to 6-membered heteroarylene. In some embodiments, A is:

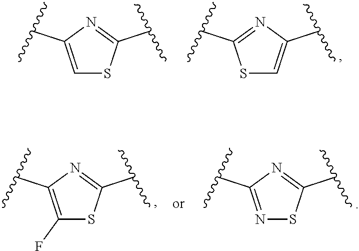 [SMILES: *c1csc(*)n1 ; *c1csc(*)n1 ; *c1nc(*)c(F)s1 ; *c1nsc(*)n1]

In some embodiments, A is

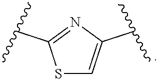 [SMILES: *c1csc(*)n1]

In some embodiments of compounds of the present invention, B is —CHR⁹—. In some embodiments, R⁹ is optionally substituted C₁-C₆ alkyl or optionally substituted 3 to 6-membered cycloalkyl. In some embodiments, R⁹ is:

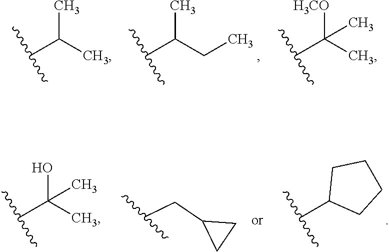

In some embodiments, R⁹ is:

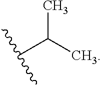 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, B is optionally substituted 6-membered arylene.

In some embodiments, B is 6-membered arylene. In some embodiments, B is:

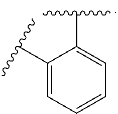 [SMILES: *c1ccccc1*]

In some embodiments B is absent.

In some embodiments of compounds of the present invention, R⁷ is methyl.

In some embodiments of compounds of the present invention, R⁸ is methyl.

In some embodiments of compounds of the present invention, R¹⁶ is hydrogen.

In some embodiments of compounds of the present invention, the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A². In some embodiments, the linker is acyclic. In some embodiments, the linker has the structure of Formula IIa:

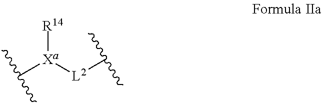

wherein X^{a} is absent or N;
R¹⁴ is absent, hydrogen or optionally substituted C₁-C₆ alkyl or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

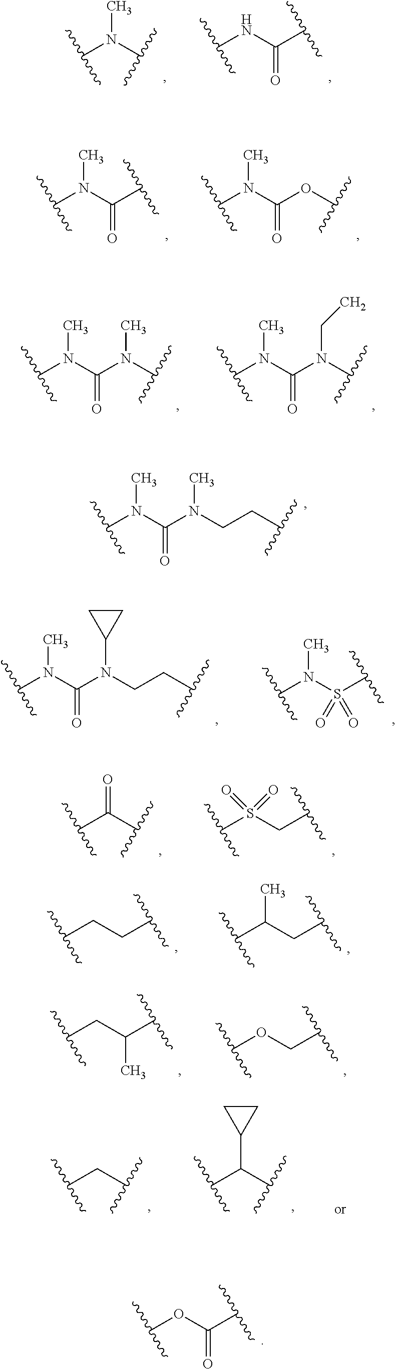

In some embodiments, L is

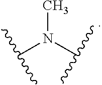 [SMILES: *N(*)C]

In some embodiments, L is

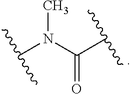 [SMILES: *C(=O)N(*)C]

In some embodiments, linker is or comprises a cyclic group. In some embodiments, linker has the structure of Formula IIb:

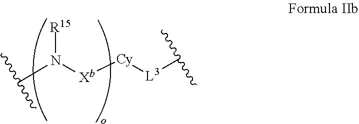

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene. In some embodiments, linker has the structure:

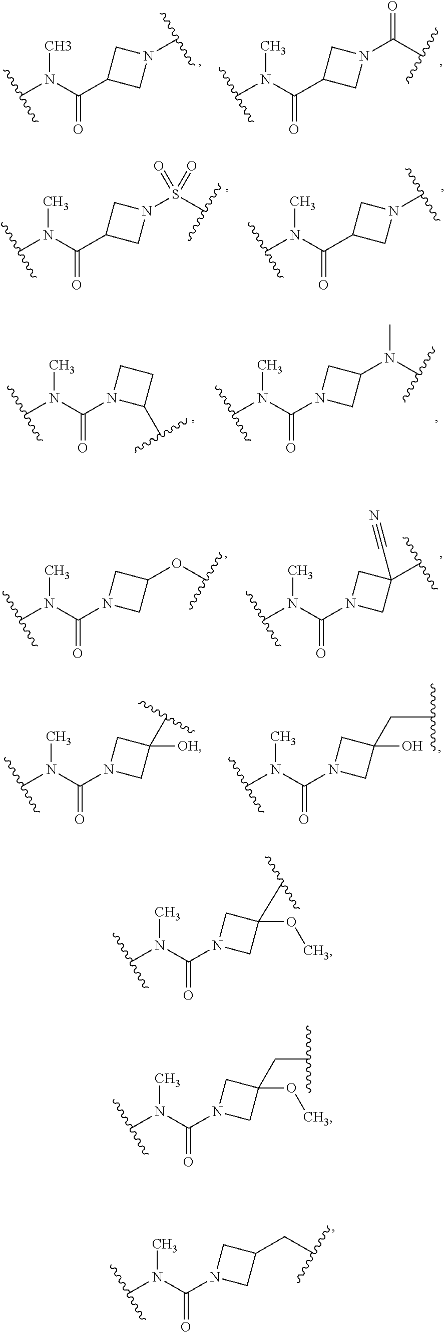

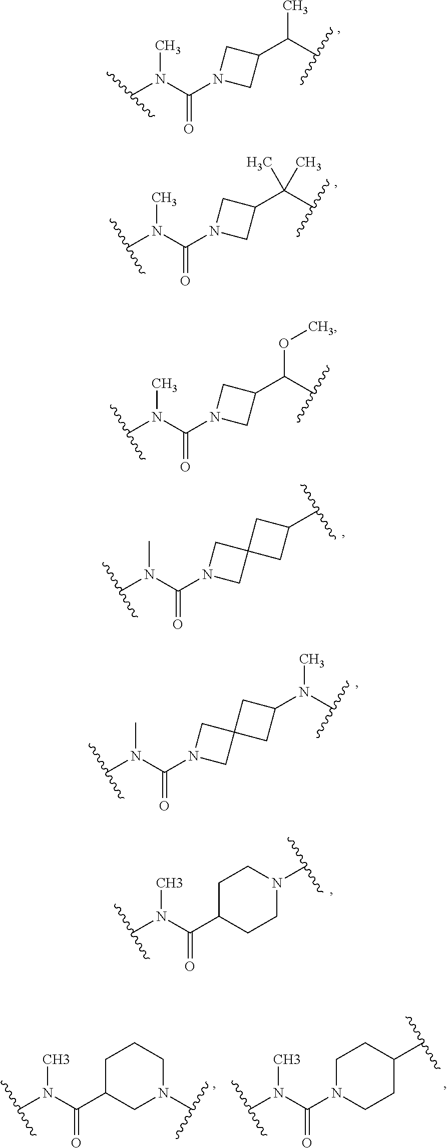

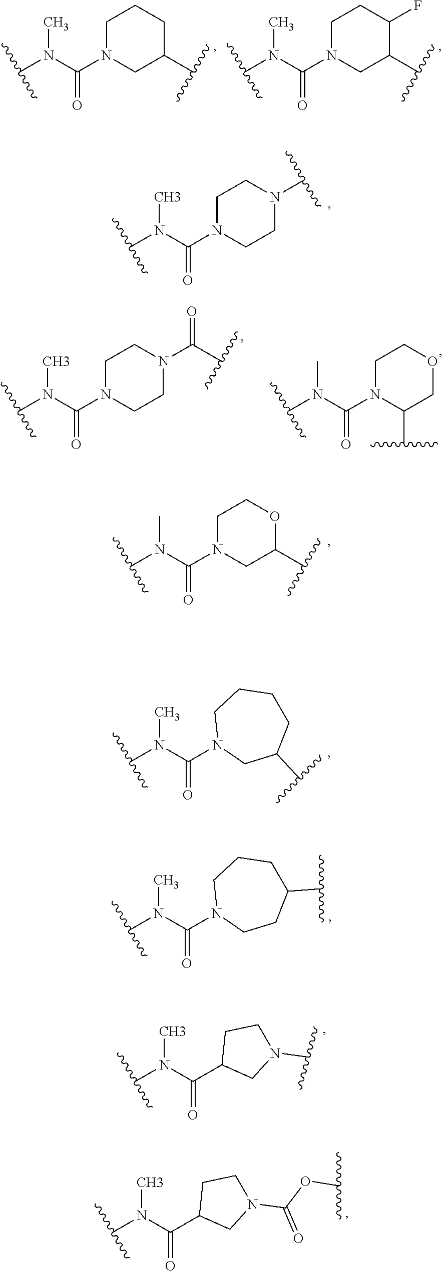

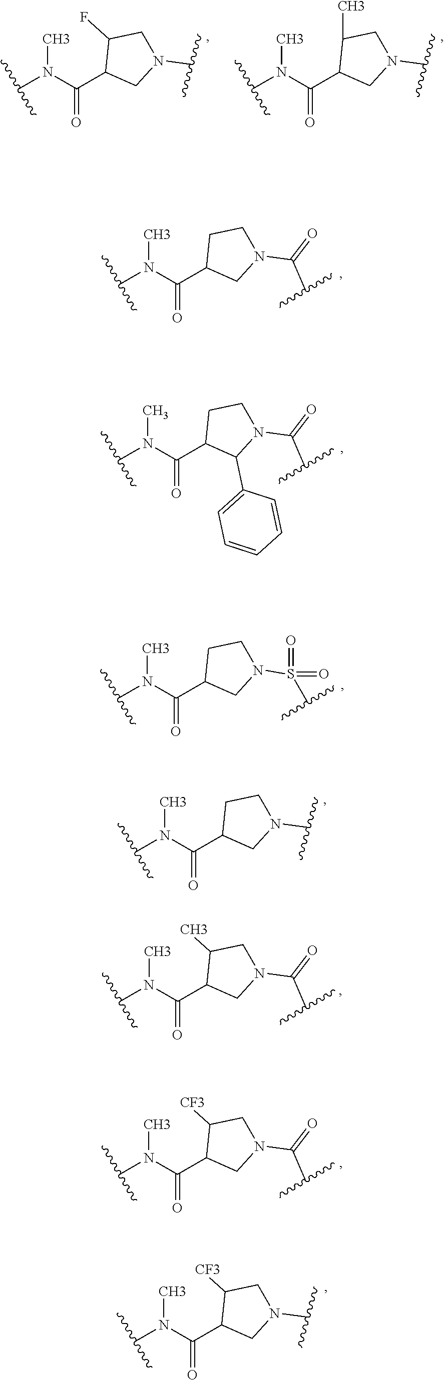

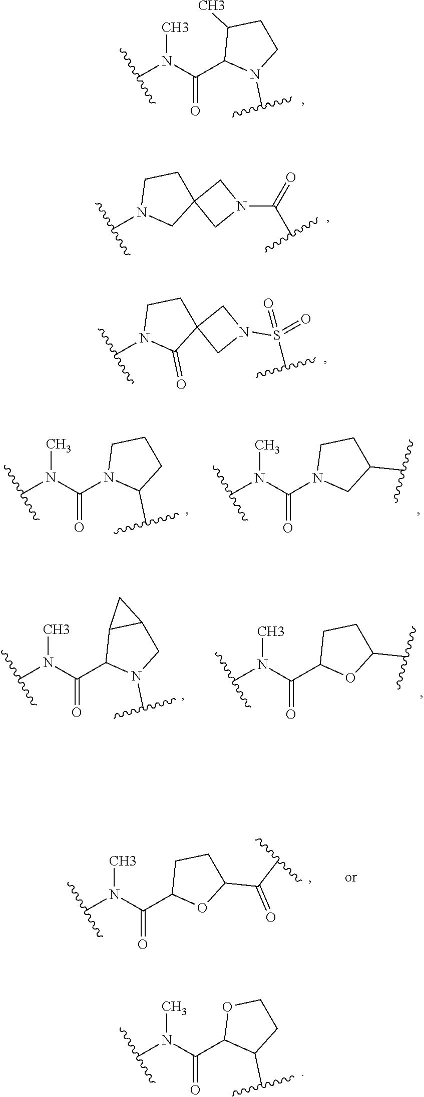

In some embodiments of compounds of the present invention, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of compounds of the present invention, W is hydrogen. In some embodiments, W is optionally substituted amino. In some embodiments, W is —NHCH₃ or —N(CH₃)₂. In some embodiments, W is optionally substituted C₁-C₄ alkoxy. In some embodiments, W is methoxy or iso-propoxy. In some embodiments, W is optionally substituted C₁-C₄ alkyl. In some embodiments, W is methyl, ethyl, iso-propyl, tert-butyl, or benzyl. In some embodiments, W is optionally substituted amido. In some embodiments, W is

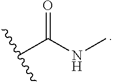 [SMILES: *C(=O)NC]

In some embodiments, W is optionally substituted amido. In some embodiments, W is

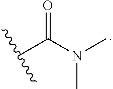 [SMILES: *C(=O)N(C)C]

In some embodiments, W is optionally substituted C₁-C₄ hydroxyalkyl. In some embodiments, W is

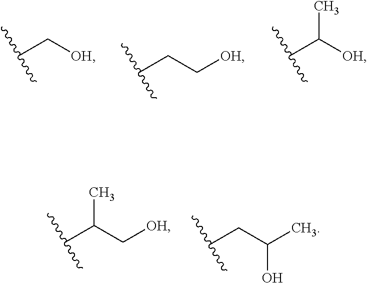

In some embodiments, W is optionally substituted C₁-C₄ aminoalkyl. In some embodiments, W is

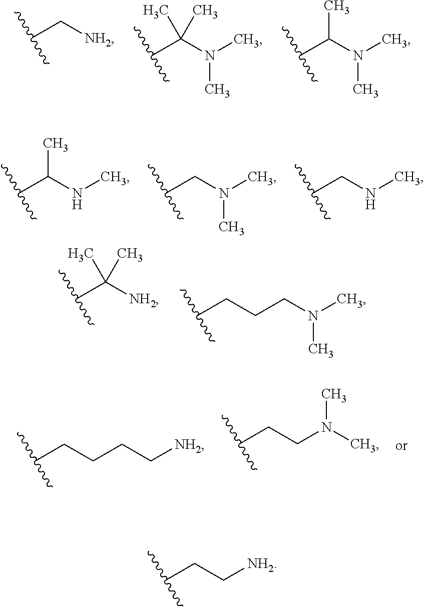

In some embodiments, W is optionally substituted C₁-C₄ haloalkyl. In some embodiments, W is

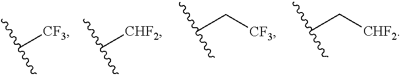

In some embodiments, W is optionally substituted C₁-C₄ guanidinoalkyl. In some embodiments, W is

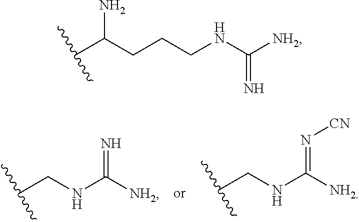 [SMILES: *C(N)CCCNC(=N)N ; *CNC(=N)N ; *CN/C(N)=N/C#N]

In some embodiments, W is C₀-C₄ alkyl optionally substituted 3 to 11-membered heterocycloalkyl. In some embodiments, W is

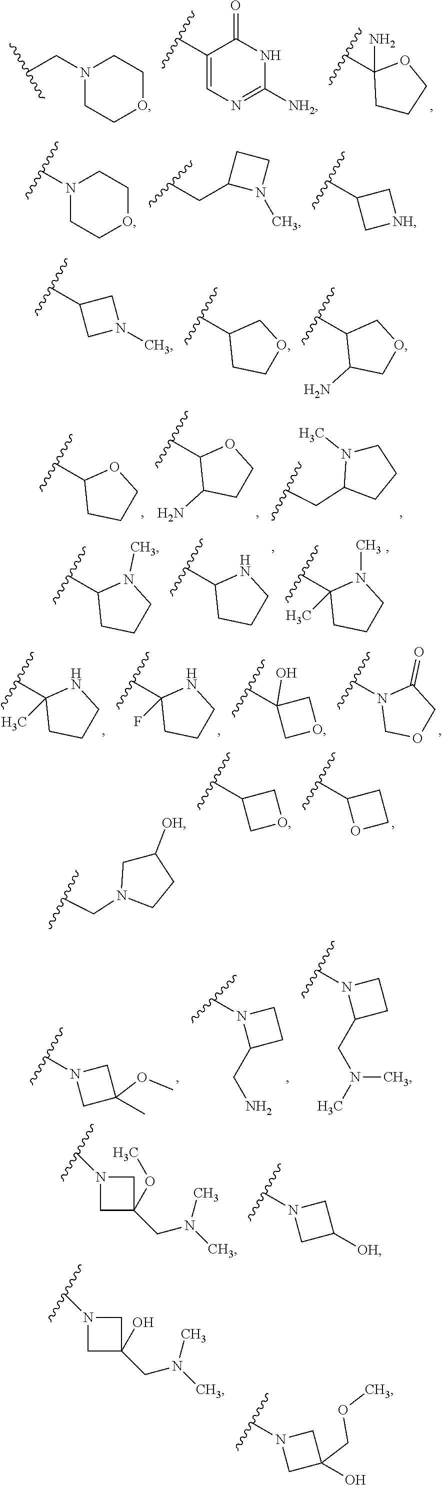

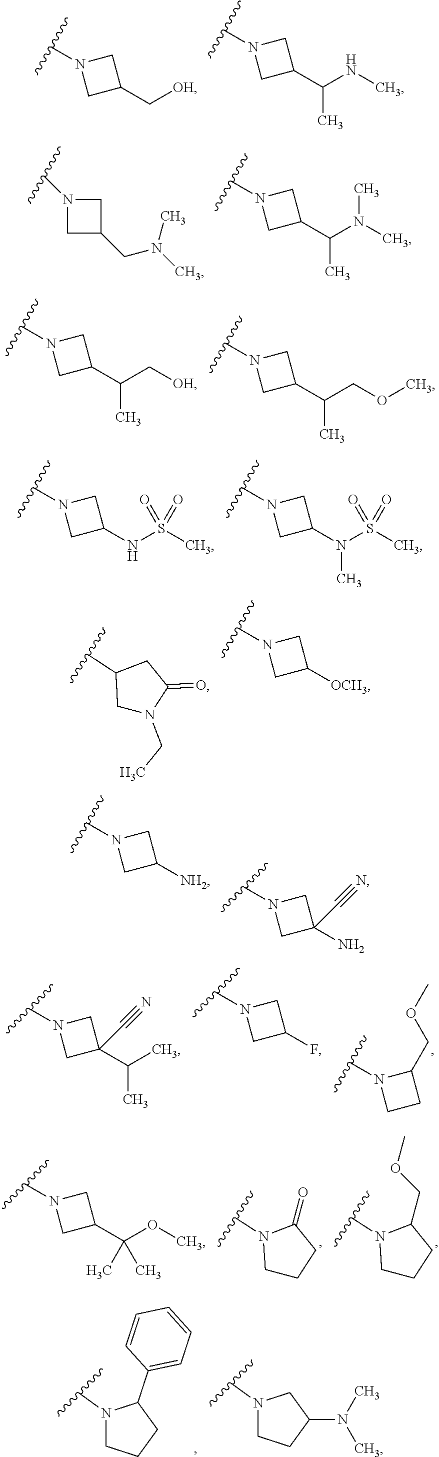

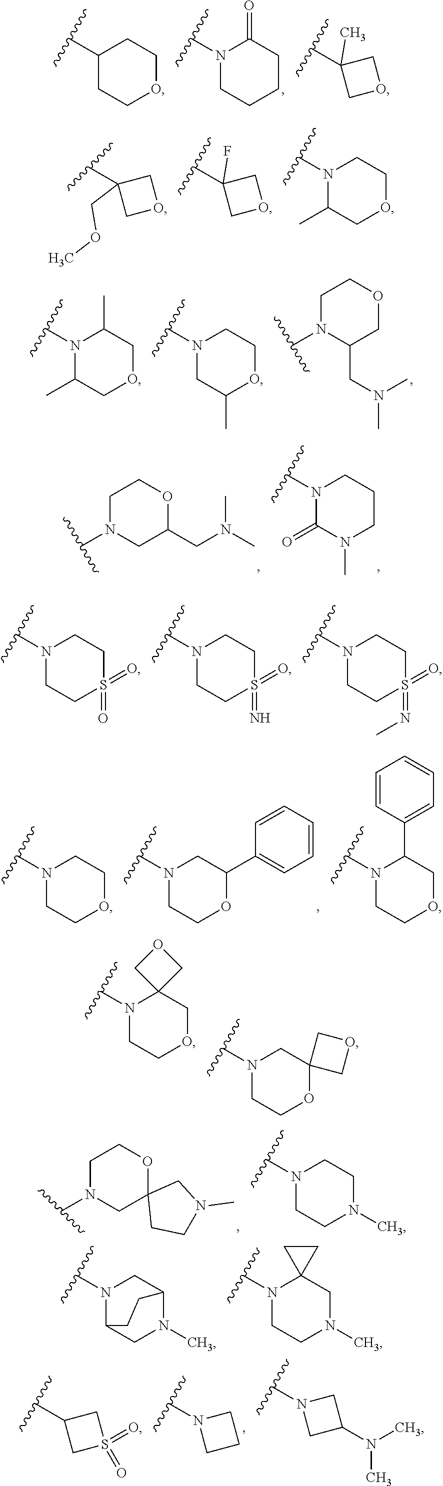

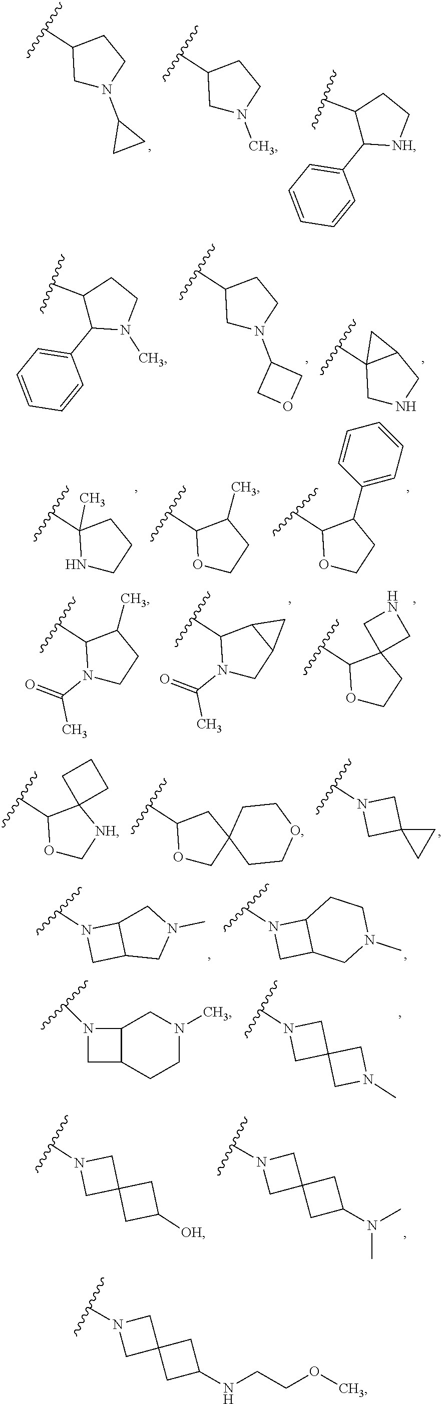

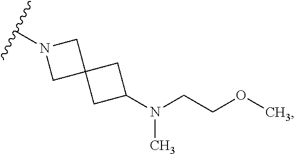 [SMILES: *N1CC2(CC(N(C)CCOC)C2)C1]

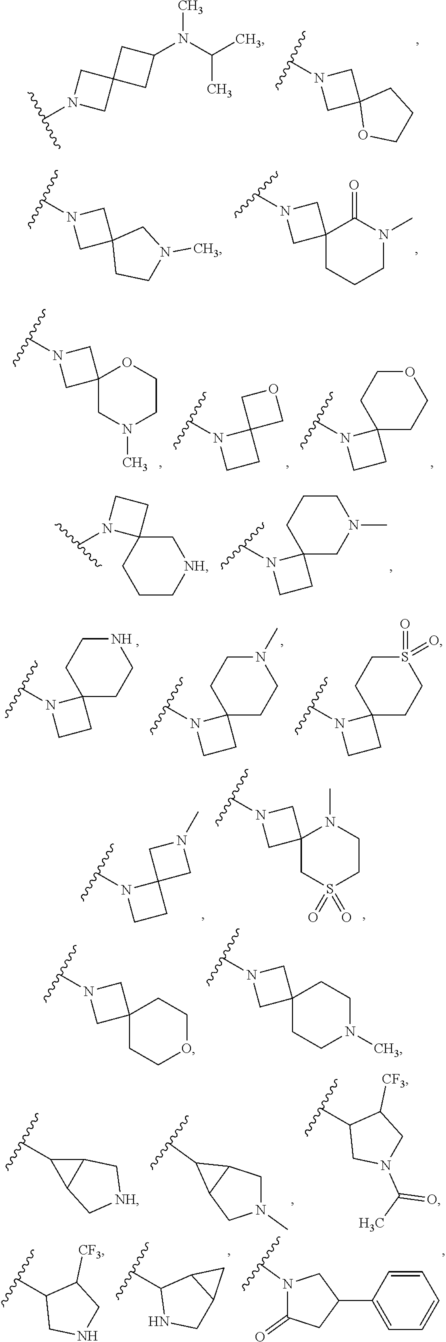

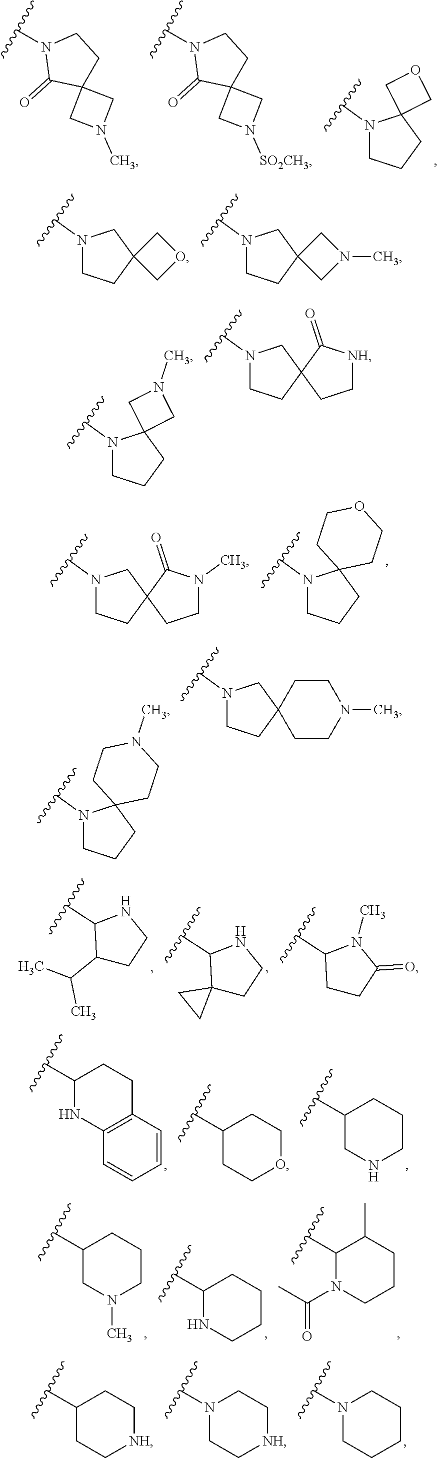

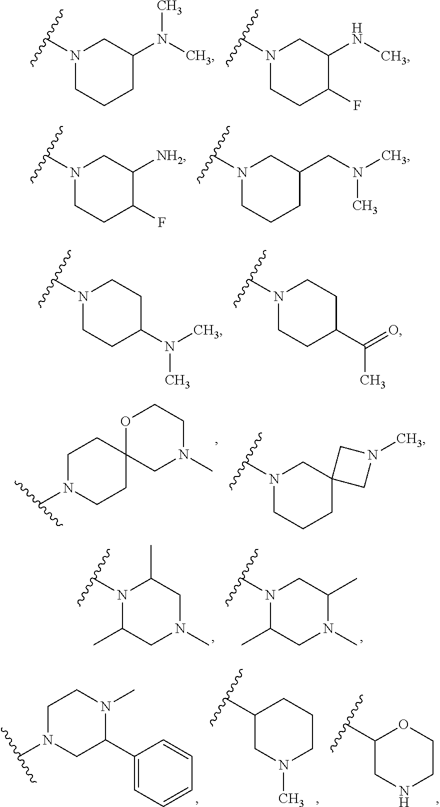

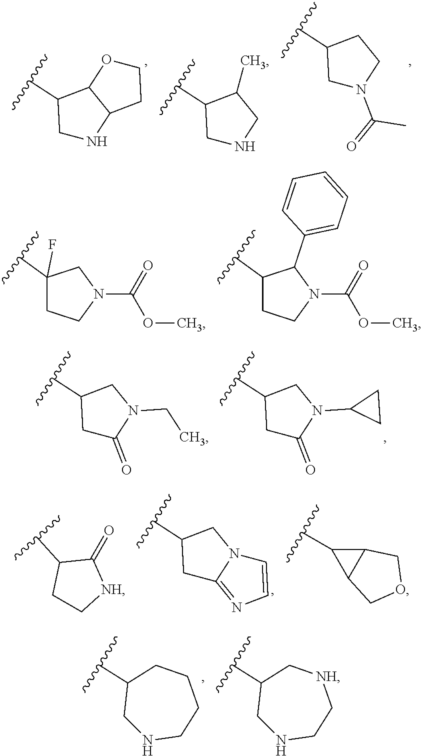

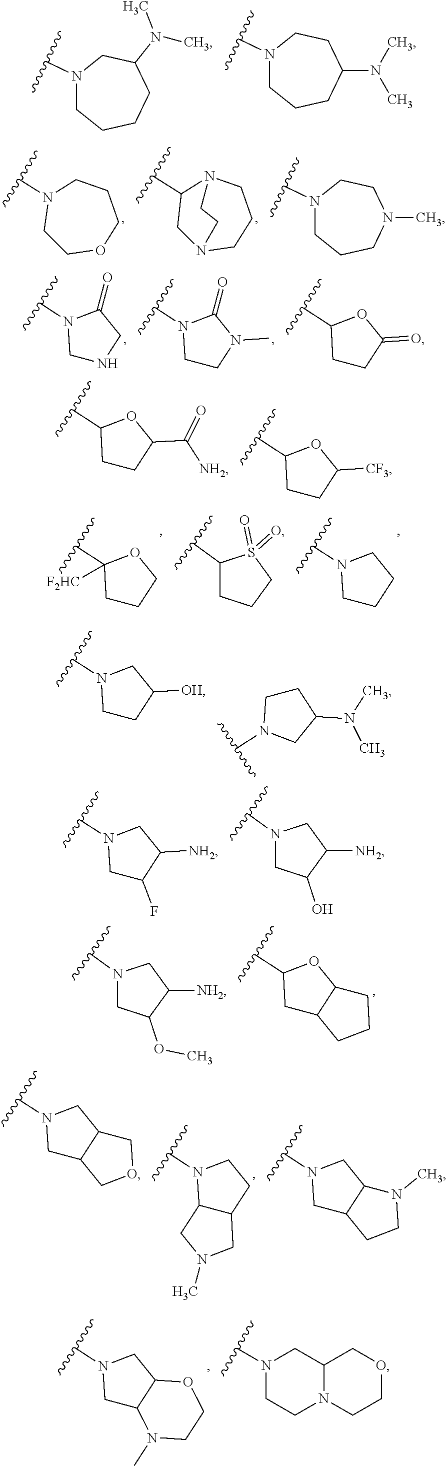

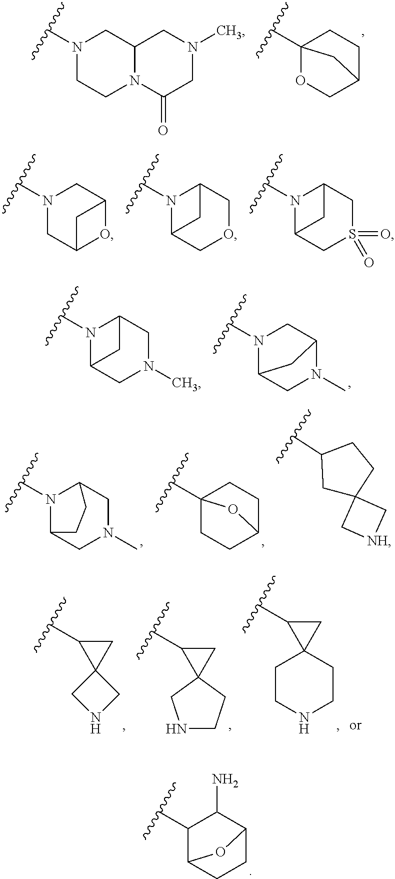

In some embodiments, W is optionally substituted 3 to 8-membered cycloalkyl. In some embodiments, W is

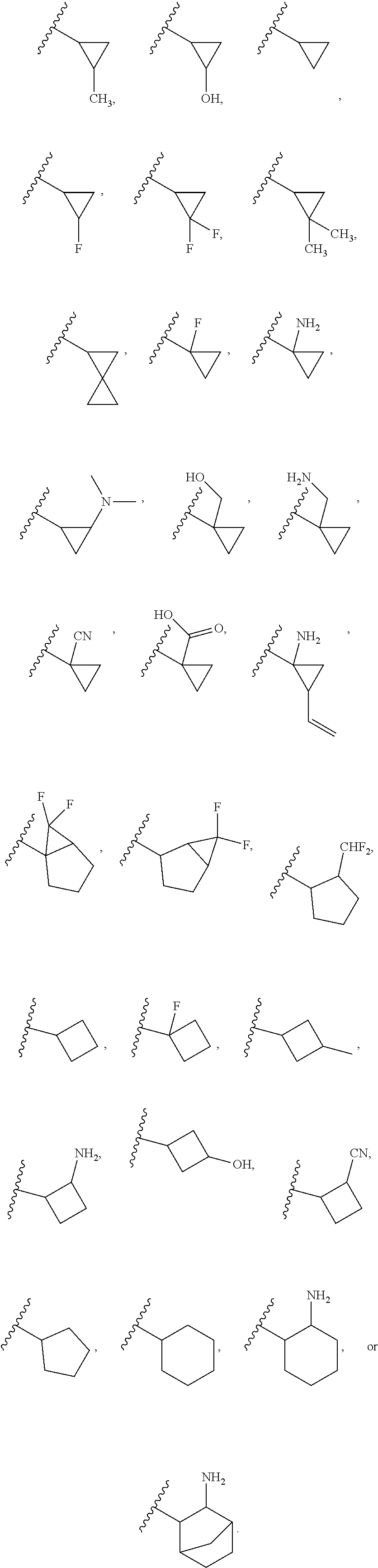

In some embodiments, W is optionally substituted 3 to 8-membered heteroaryl. In some embodiments, W is

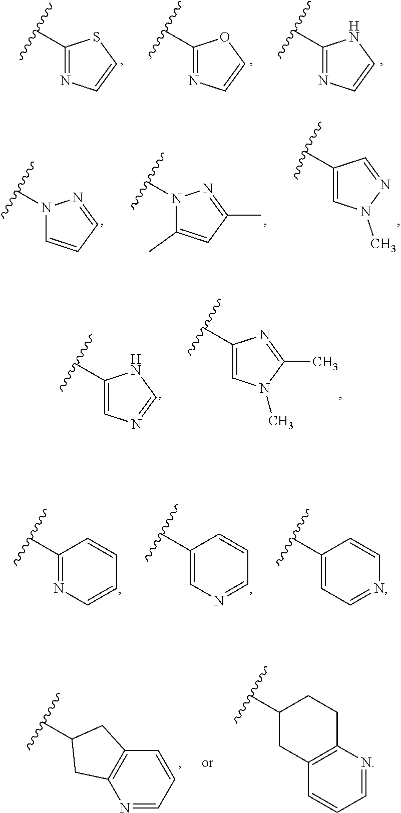

In some embodiments, W is optionally substituted 6- to 10-membered aryl (e.g., phenyl, 4-hydroxy-phenyl, or 2,4-methoxyphenyl).

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 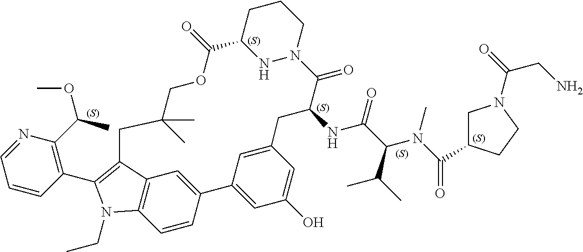 |
| A2 | 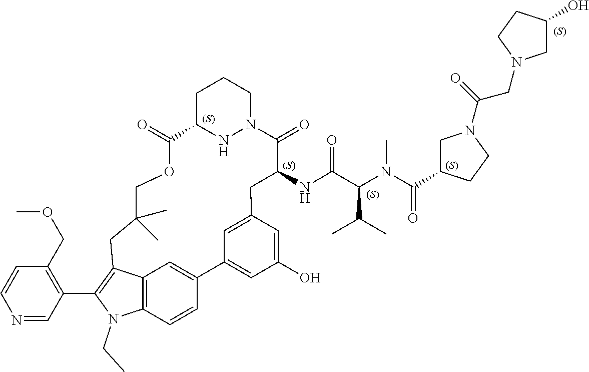 |
| A3 | 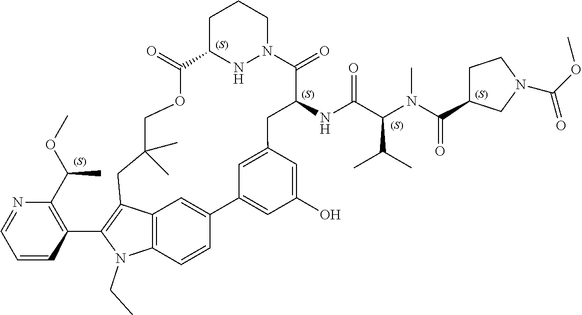 |
| A4 | 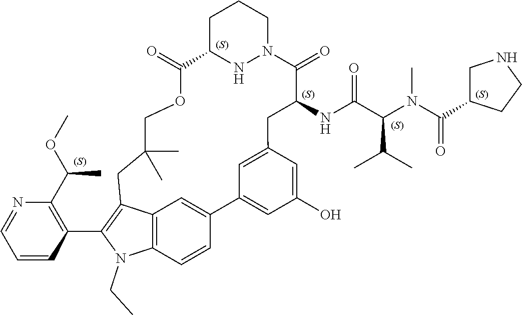 |
| A5 | 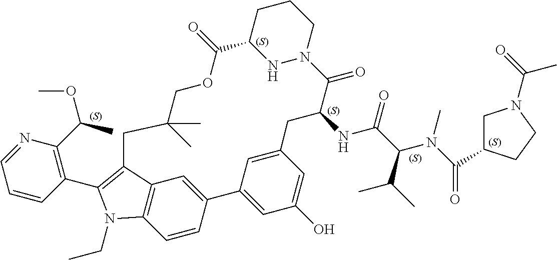 |
| A6 | 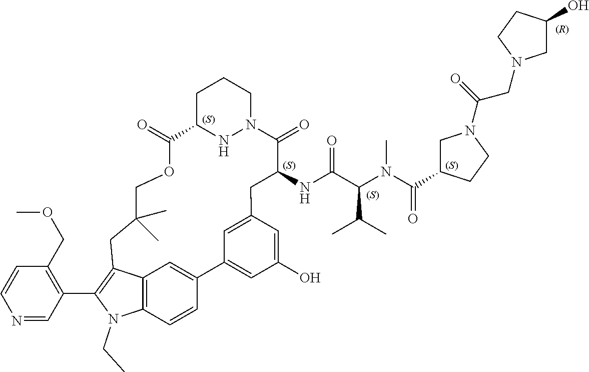 |
| A7 | 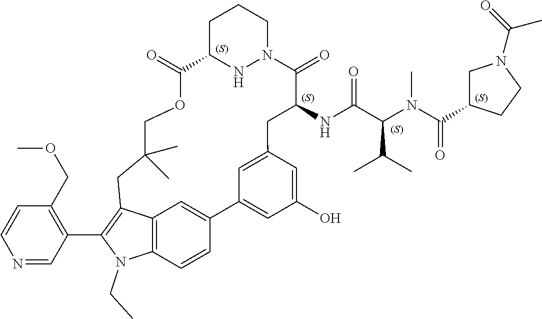 |
| A8 | 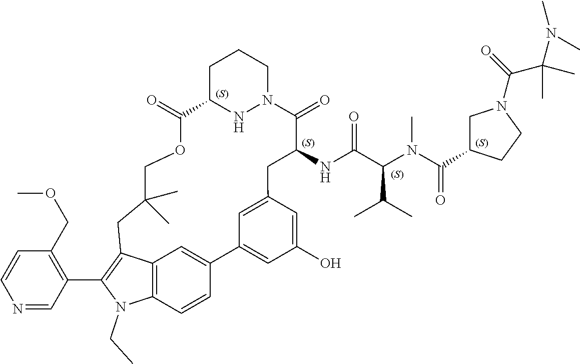 |
| A9 | 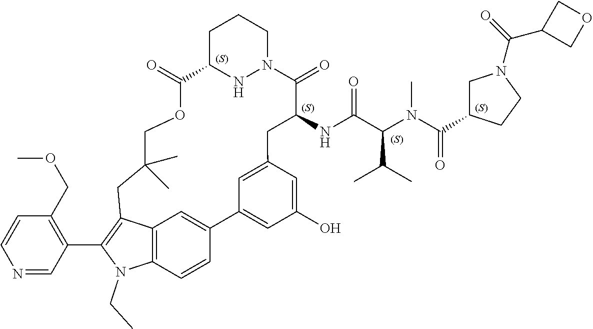 |
| A10 | 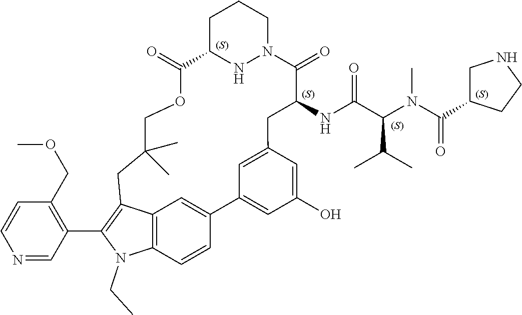 |
| A11 | 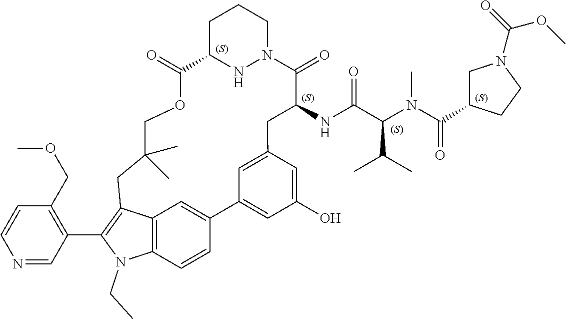 |
| A12 | 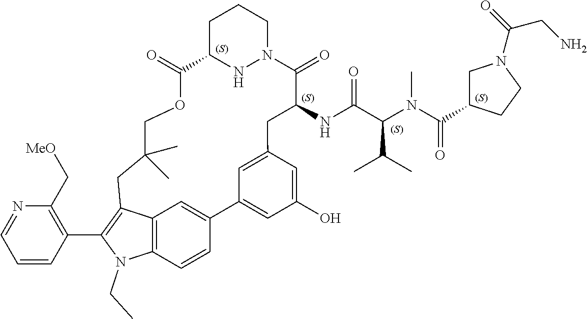 |
| A13 | 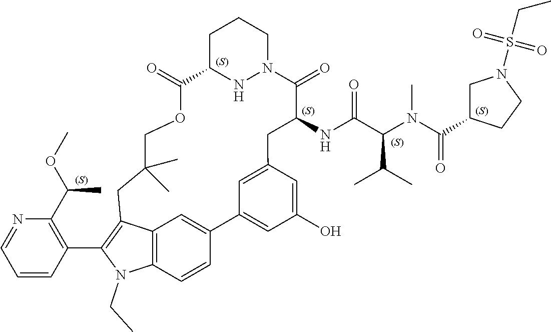 |
| A14 | 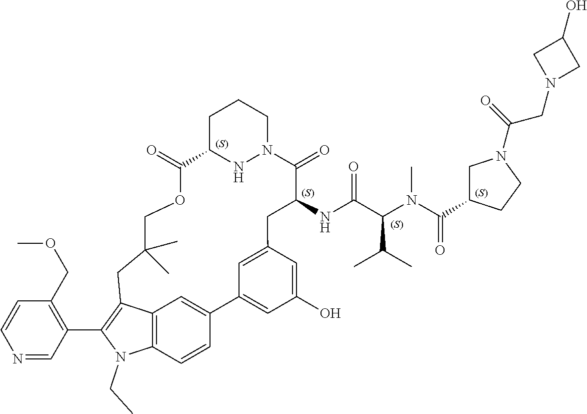 |
| A15 | 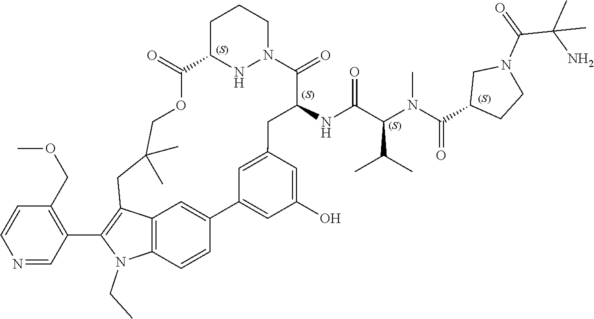 |
| A16 | 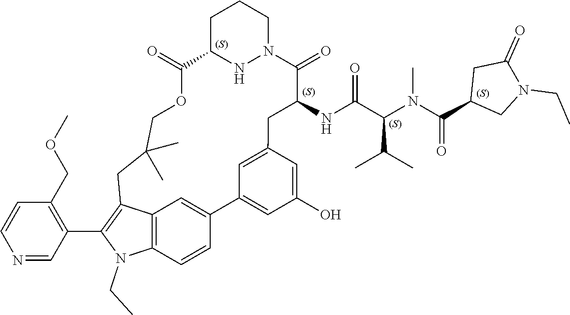 |
| A17 | 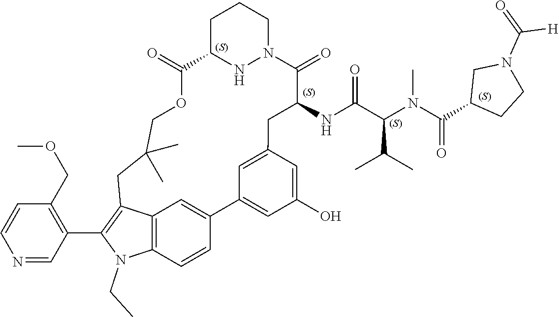 |
| A18 | 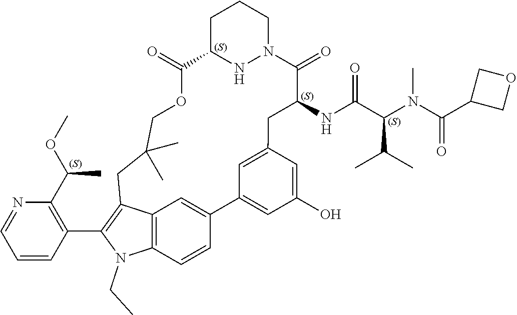 |
| A19 | 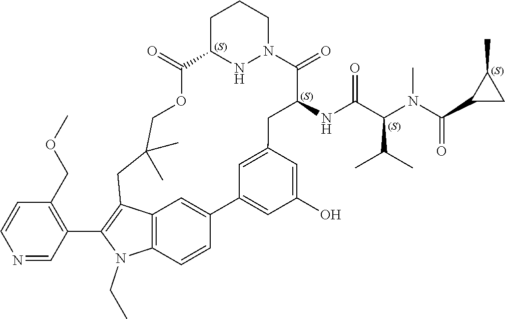 |
| A20 | 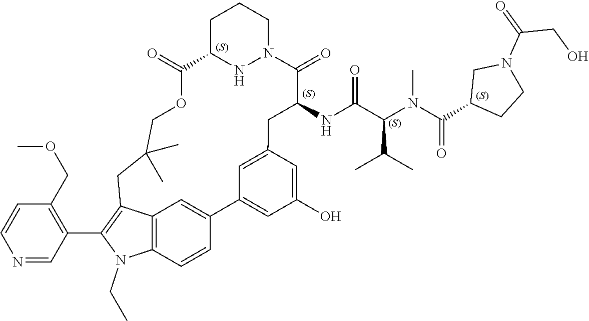 |
| A21 | 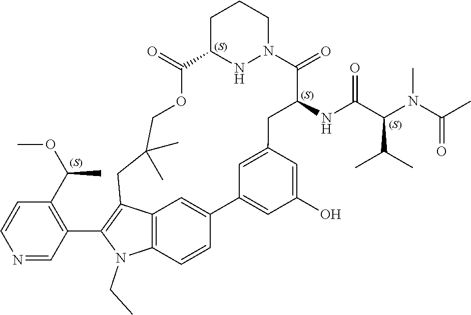 |
| A22 | 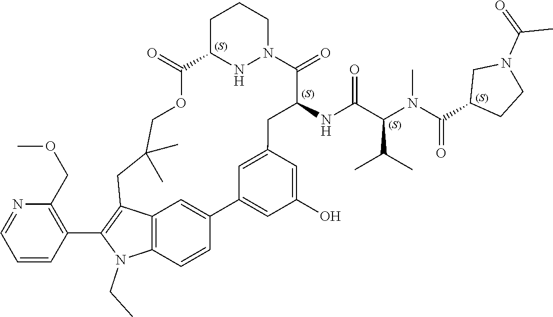 |
| A23 | 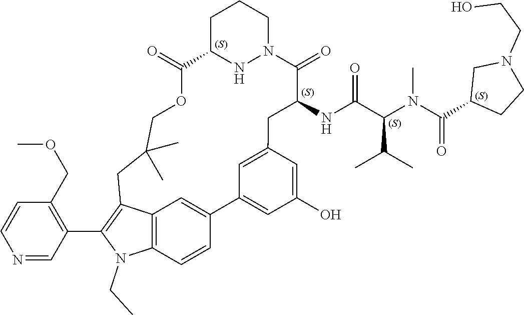 |
| A24 | 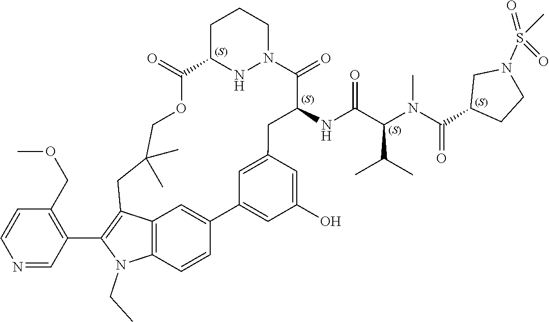 |
| A25 | 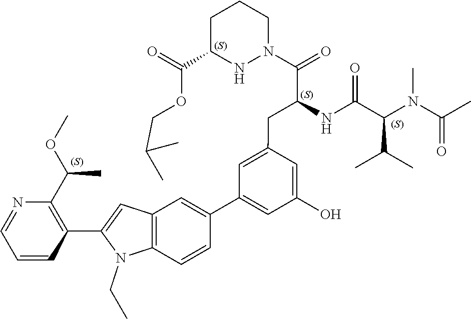 |
| A26 | 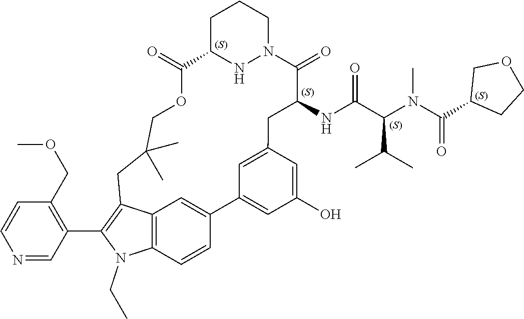 |
| A27 | 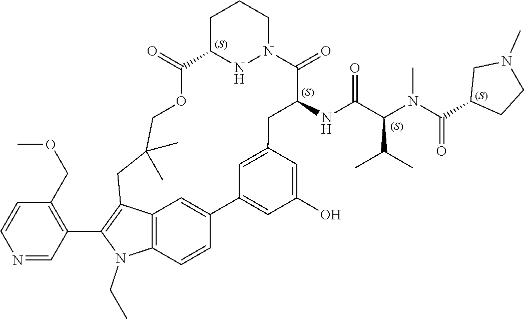 |
| A28 | 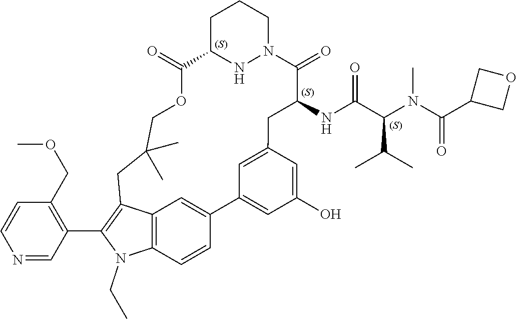 |
| A29 | 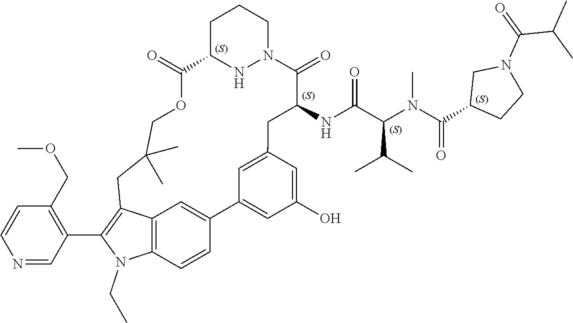 |
| A30 | 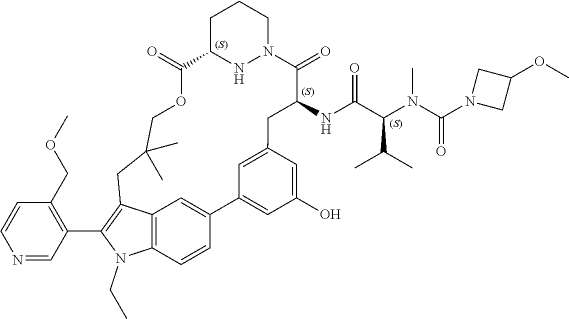 |
| A31 | 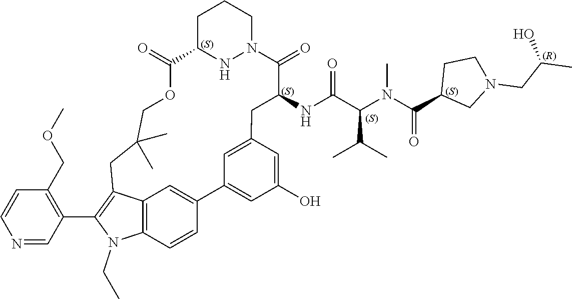 |
| A32 | 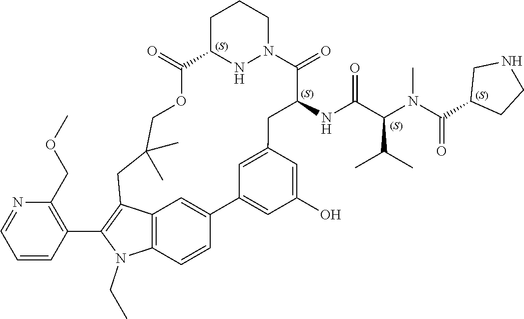 |
| A33 | 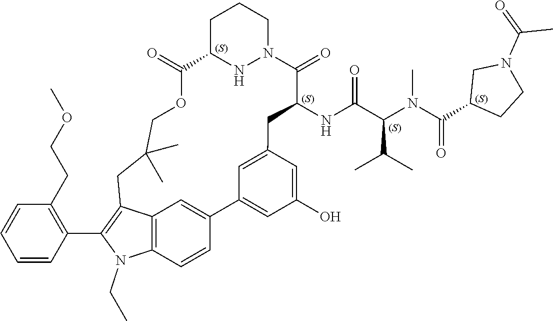 |
| A34 | 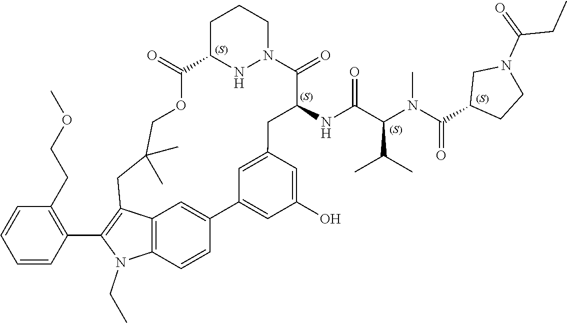 |
| A35 | 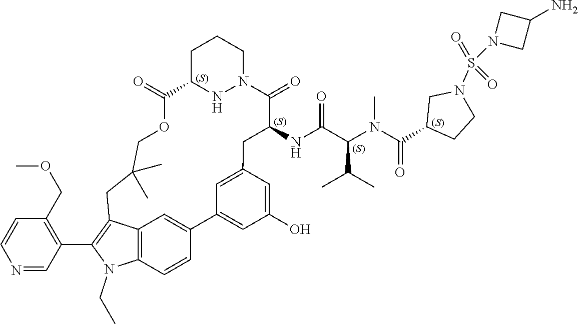 |
| A36 | 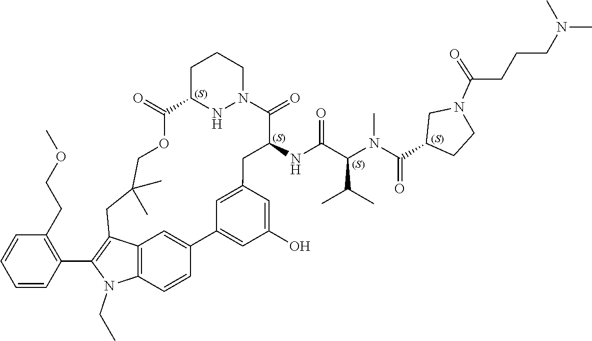 |
| A37 | 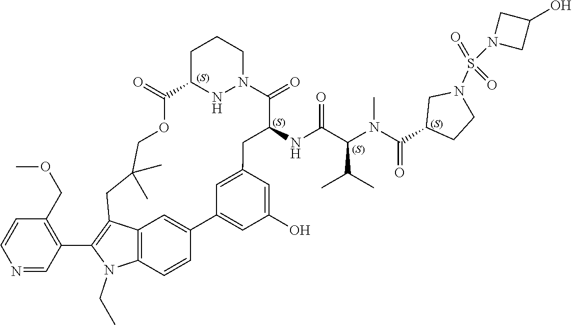 |
| A38 | 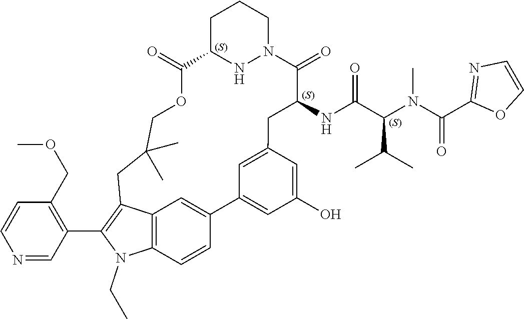 |
| A39 | 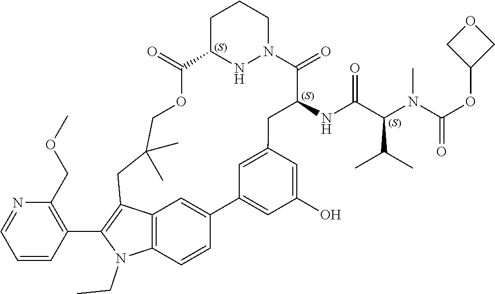 |
| A40 | 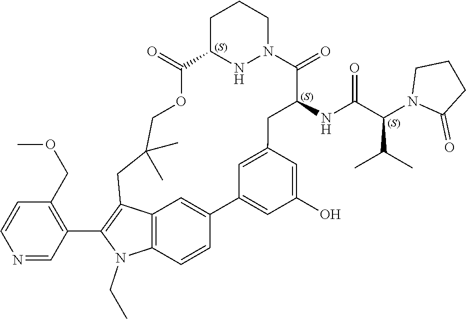 |
| A41 | 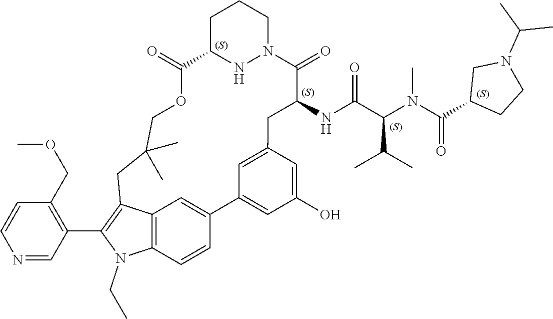 |
| A42 | 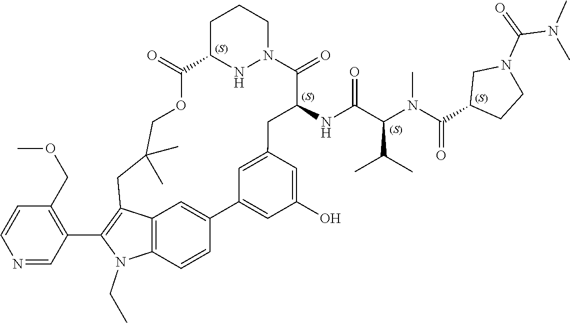 |
| A43 | 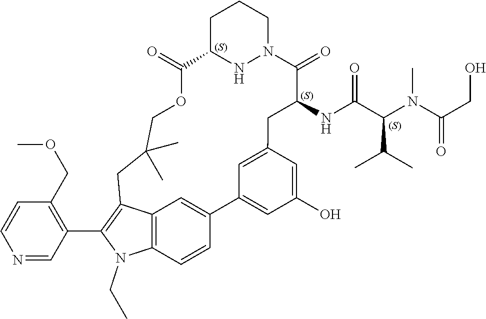 |
| A44 | 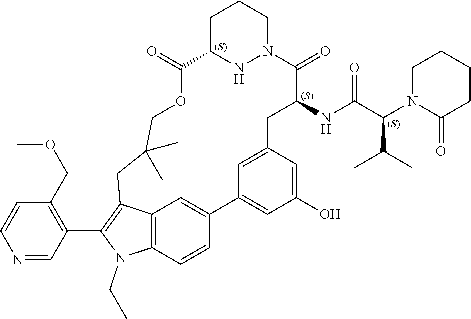 |
| A45 | 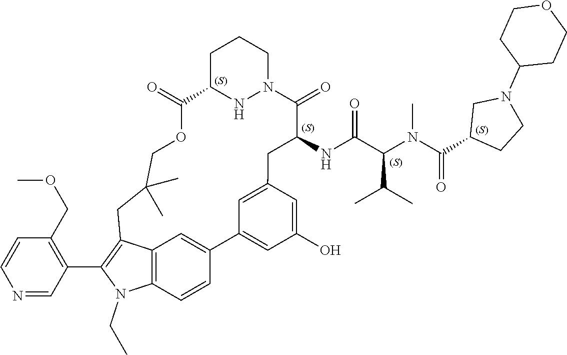 |
| A46 | 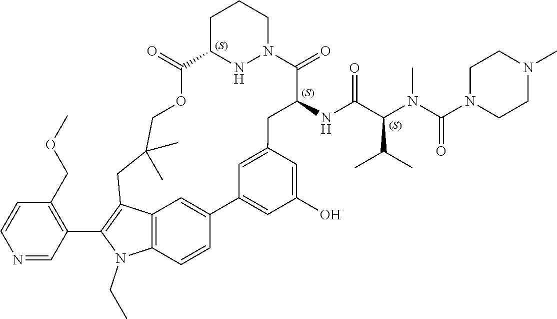 |
| A47 | 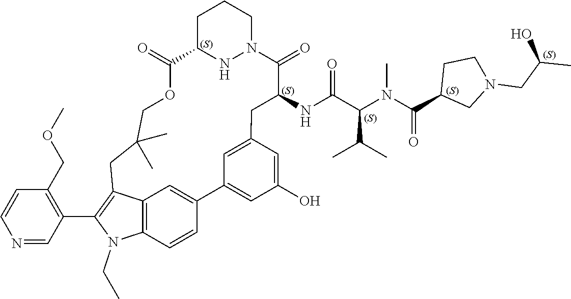 |
| A48 | 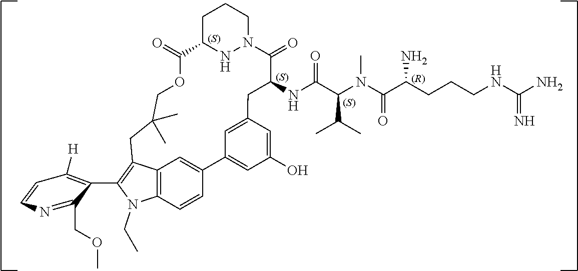 |
| A49 | 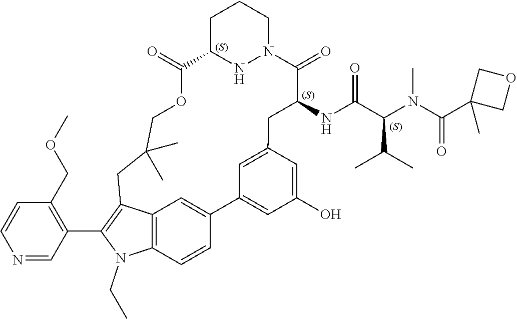 |
| A50 | 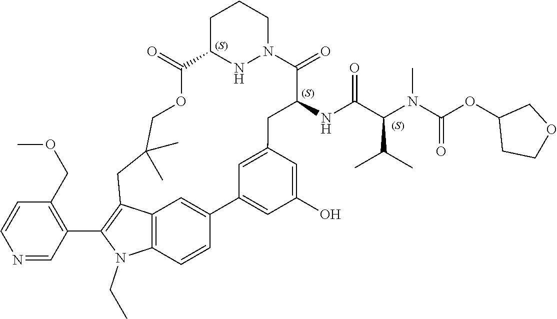 |
| A51 | 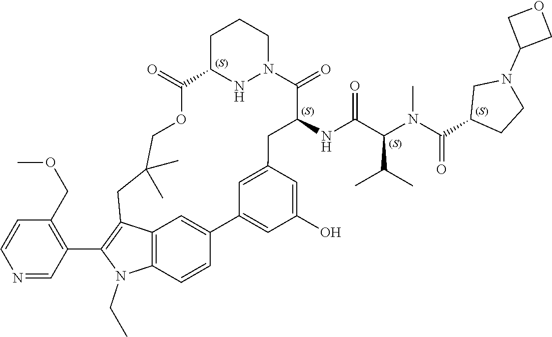 |
| A52 | 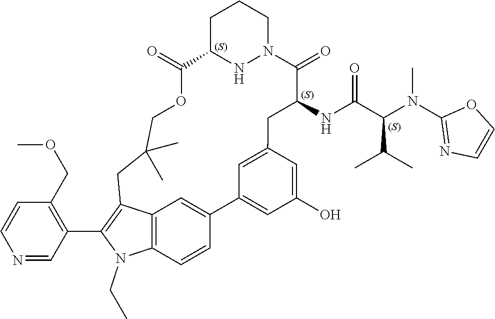 |
| A53 | 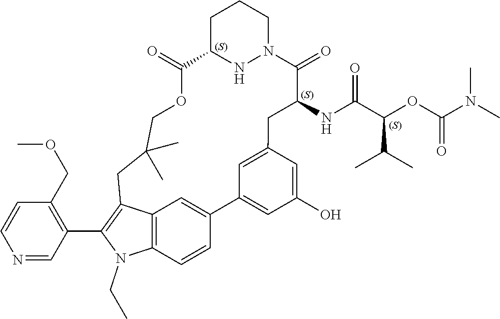 |
| A54 | 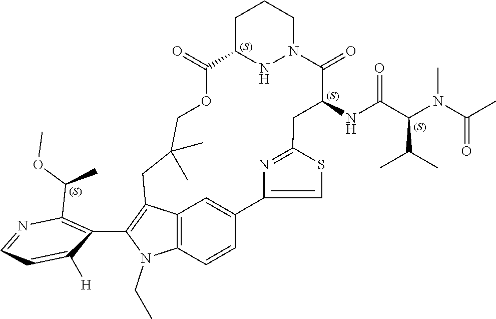 |
| A55 | 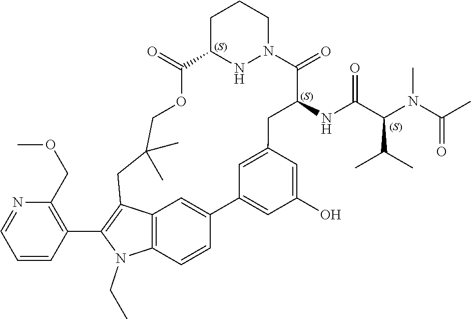 |
| A56 | 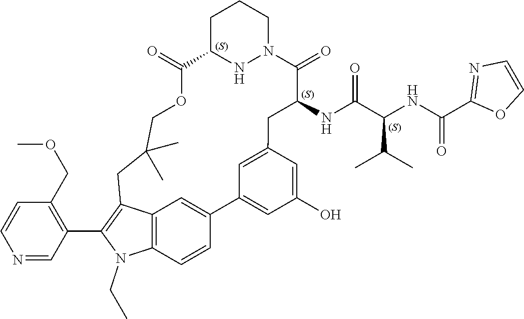 |
| A57 | 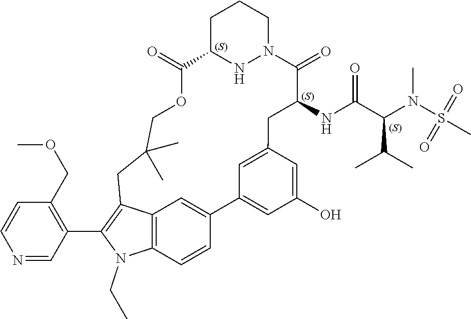 |
| A58 | 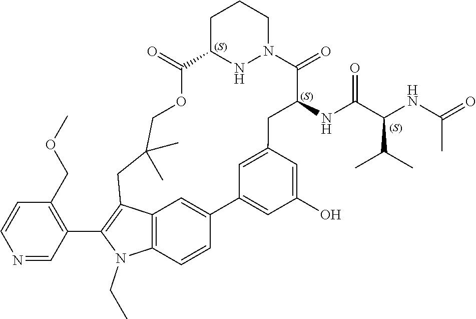 |
| A59 | 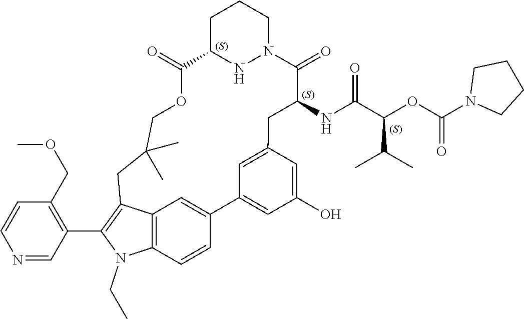 |
| A60 | 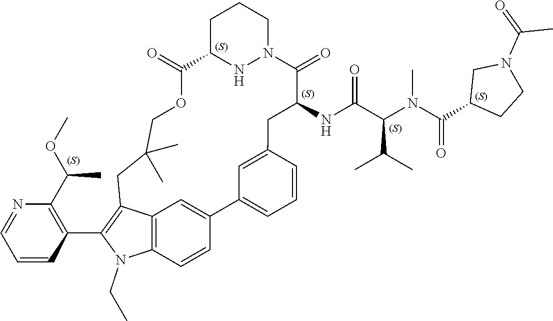 |
| A61 | 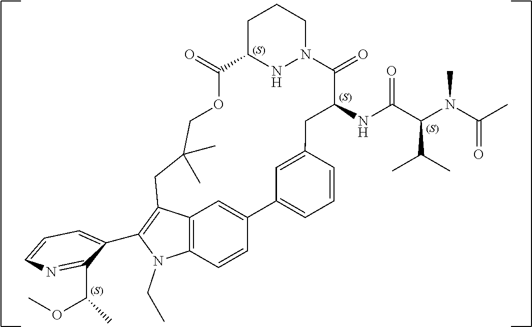 |
| A62 | 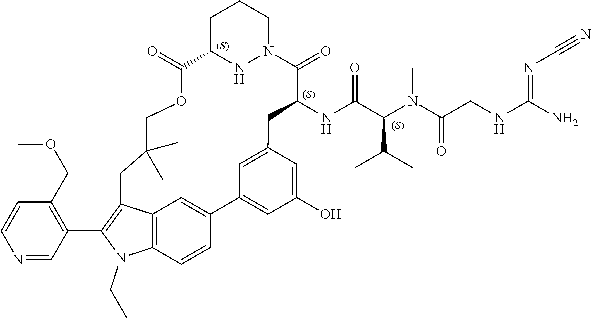 |
| A63 | 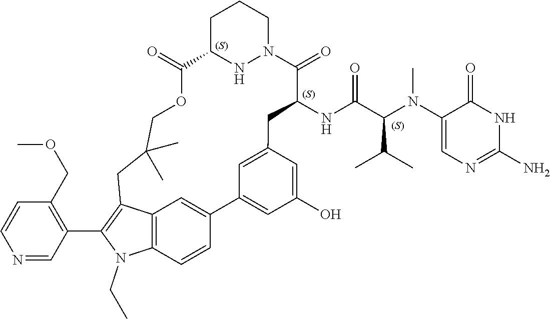 |
| A64 | 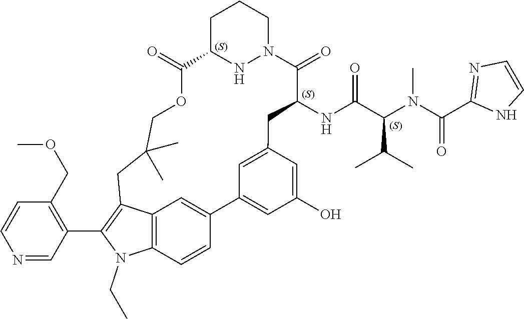 |
| A65 | 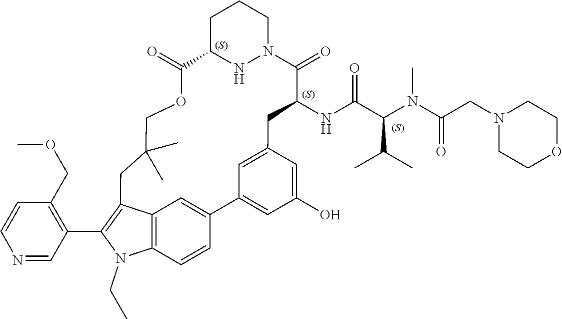 |
| A66 | 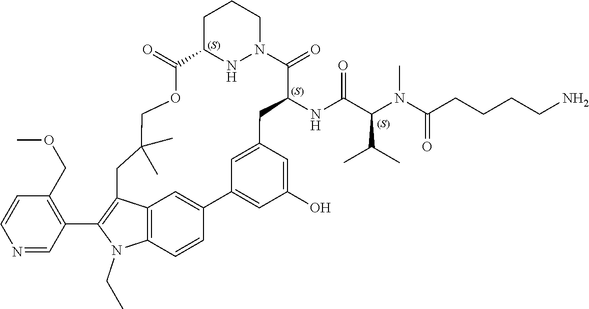 |
| A67 | 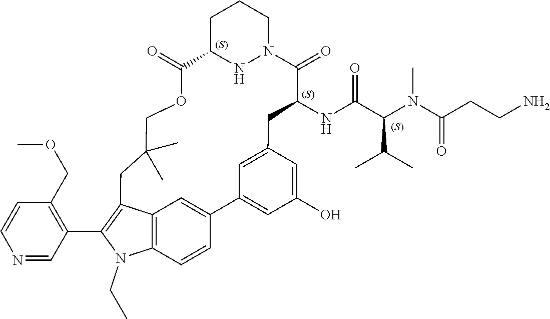 |
| A68 | 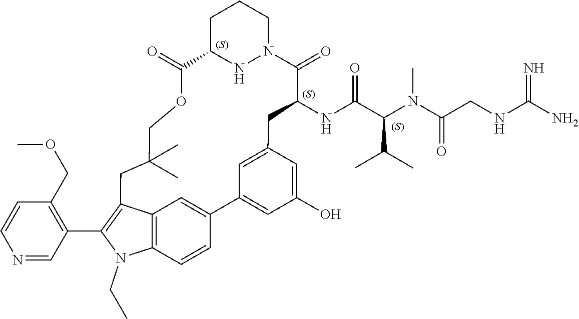 |
| A69 | 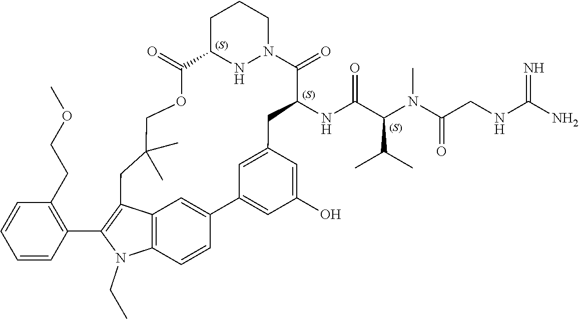 |
| A70 | 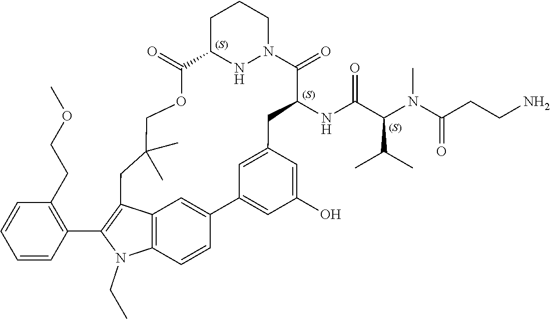 |
| A71 | 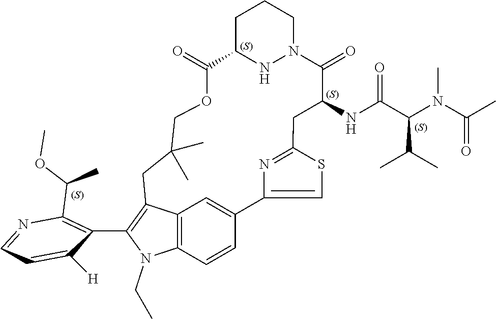 |
| A72 | 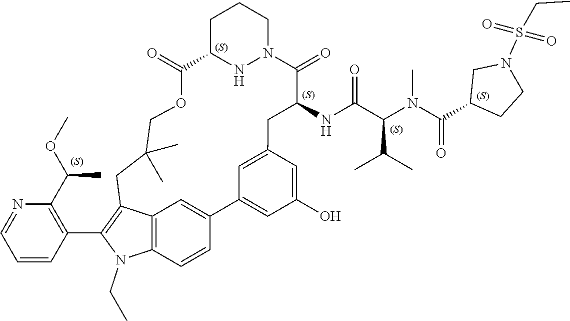 |
| A73 | 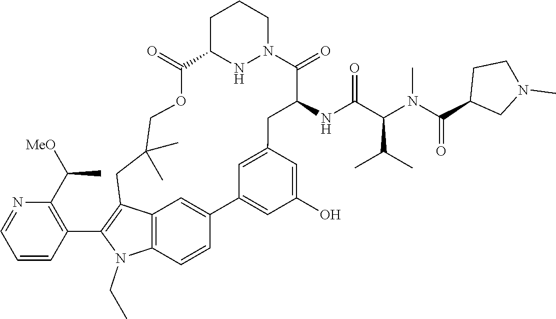 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A74 | 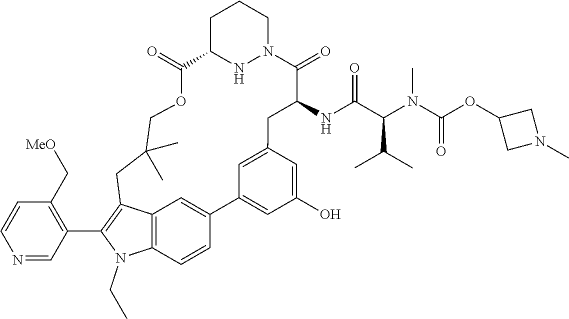 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| A75 | 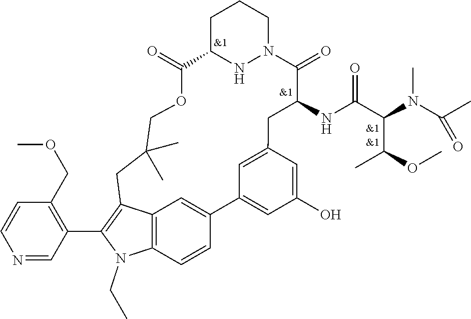 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| A76 | 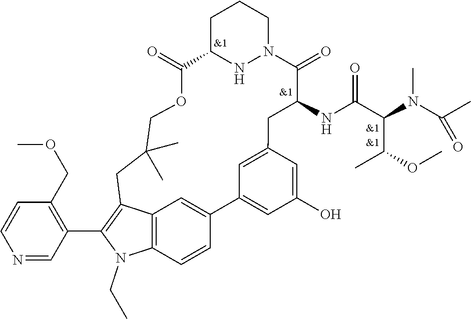 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| A77 | 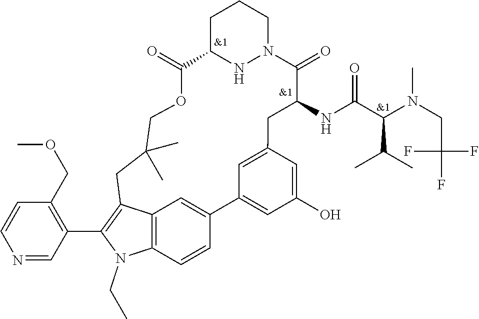 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| A78 | 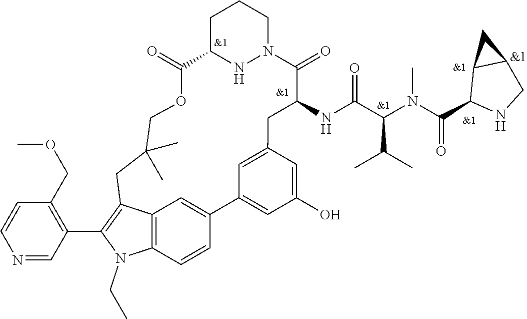 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| A79 | 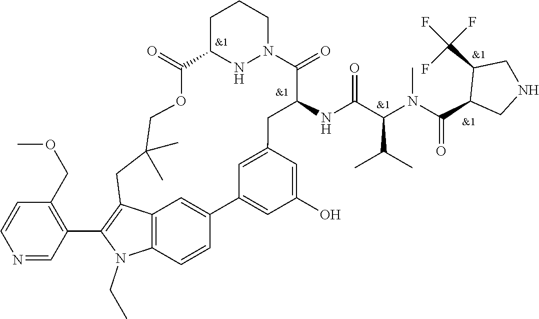 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| A80 | 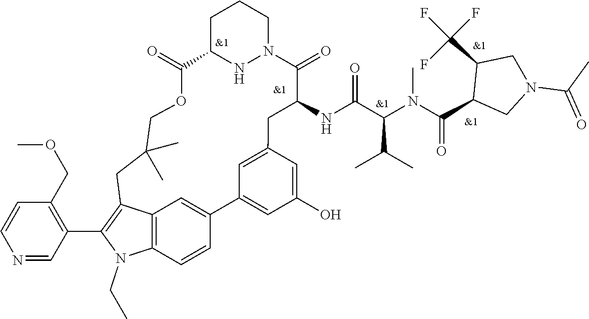 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| A81 | 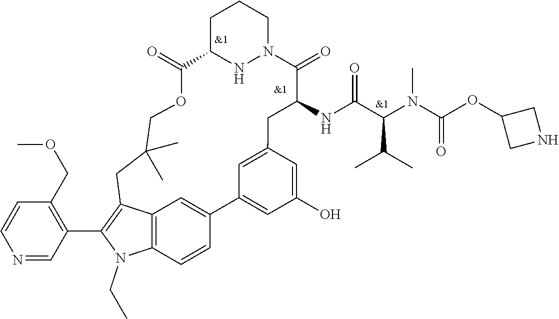 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| A82 | 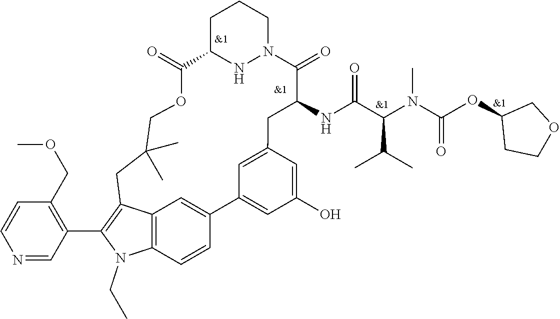 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| A83 | 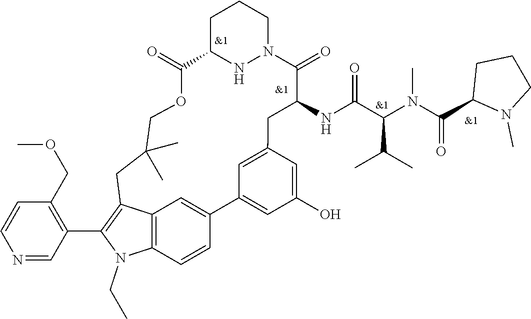 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCN1C)C(=O)N1CCC[C@H](N1)C(=O)OCC(C)(C)C2] |
| A84 | 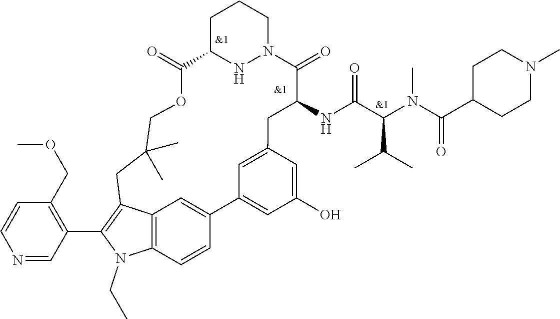 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| A85 | 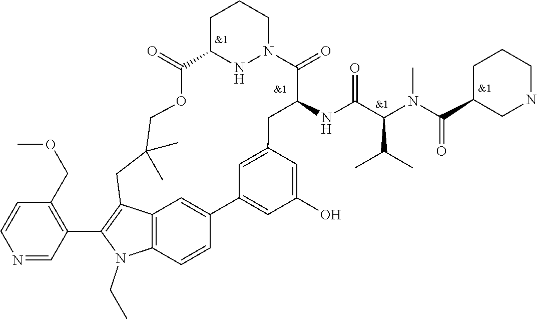 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCNC1)C(=O)N1CCC[C@H](N1)C(=O)OCC(C)(C)C2] |
| A86 | 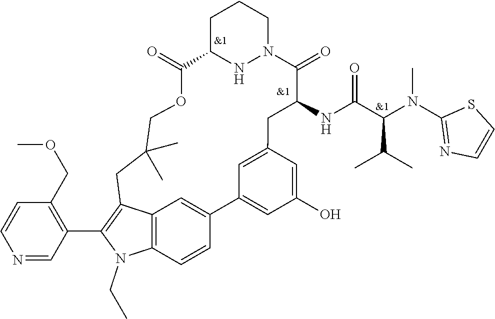 [SMILES: CCn1c(-c2cnccc2COC)c2c3cc(ccc31)-c1cc(O)cc(c1)C[C@H](NC(=O)[C@H](C(C)C)N(C)c1nccs1)C(=O)N1CCC[C@H](N1)C(=O)OCC(C)(C)C2] |
| A87 | 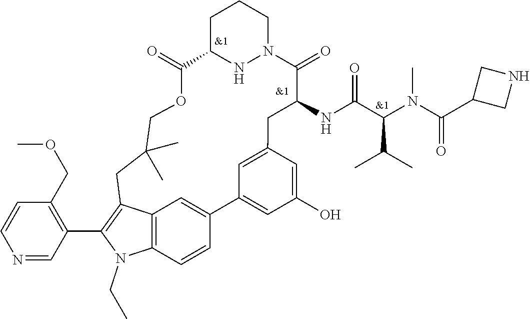 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| A88 | 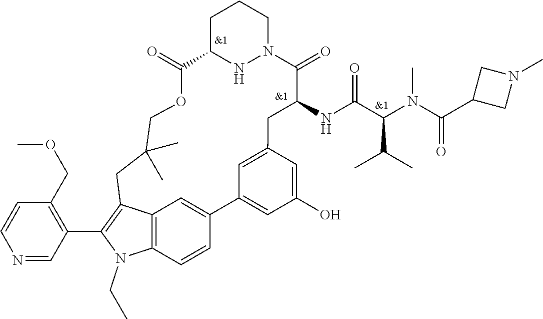 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| A89 | 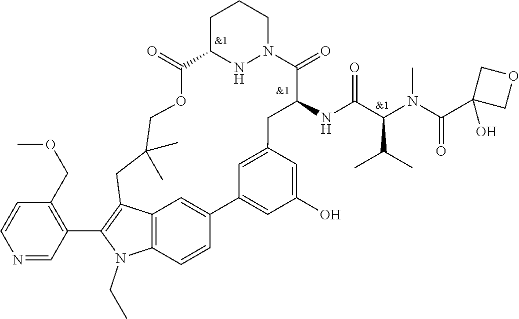 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| A90 | 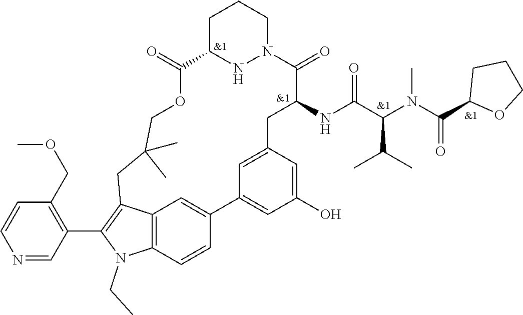 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCO1)C(=O)N1CCC[C@H](N1)C(=O)OCC(C)(C)C2] |
| A91 | 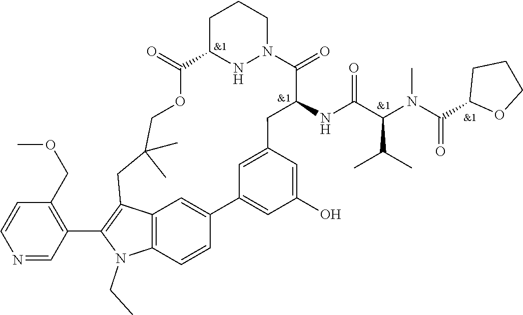 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| A92 | 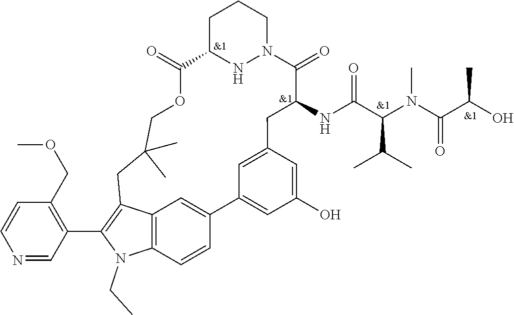 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| A93 | 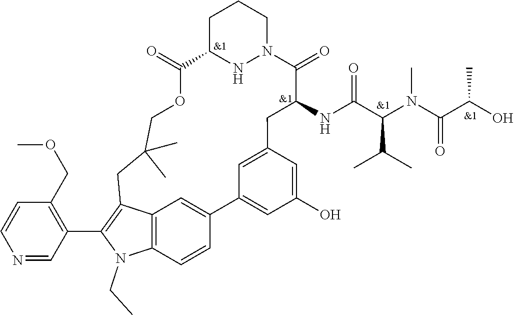 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| A94 | 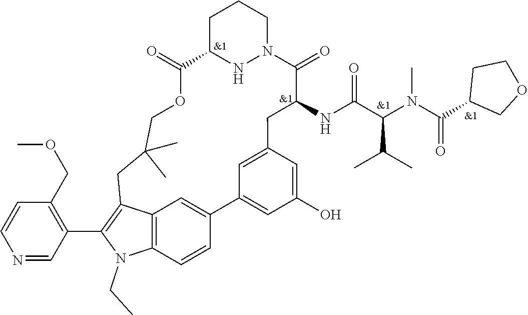 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| A95 | 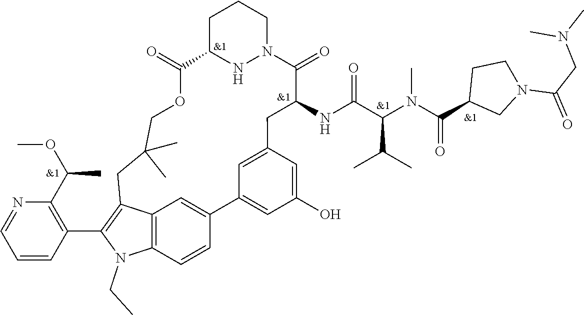 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A96 | 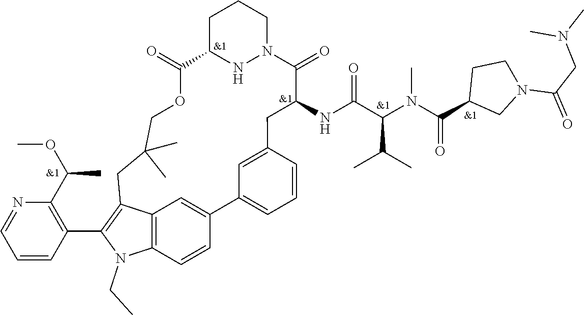 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A97 | 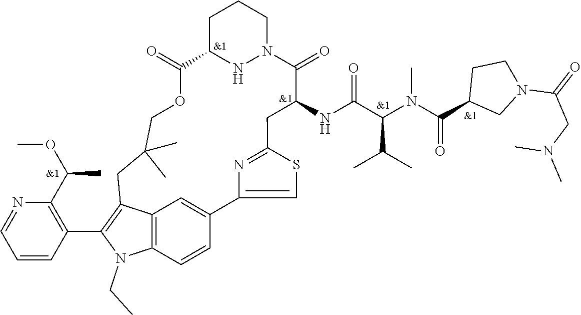 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| A98 | 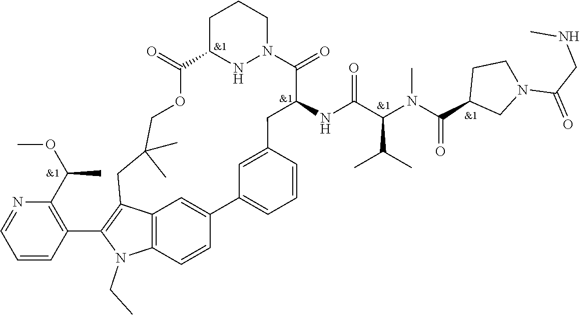 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A99 | 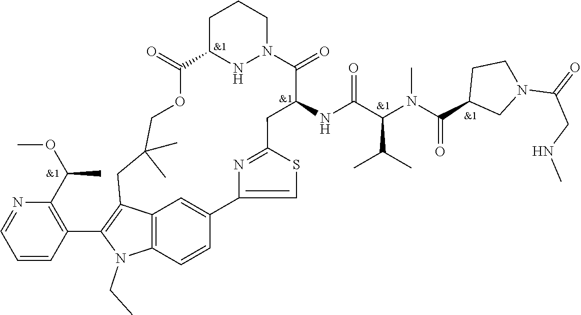 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A100 | 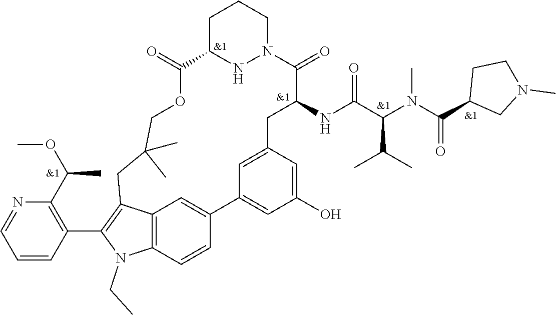 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A101 | 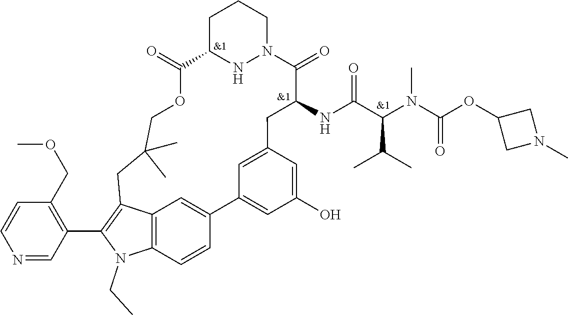 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| A102 | 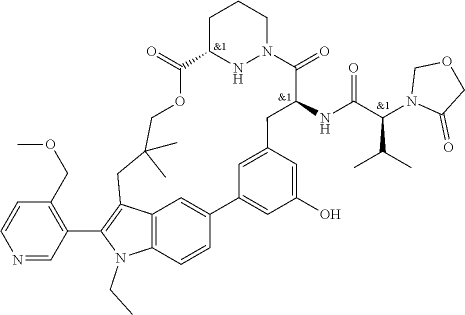 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| A103 | 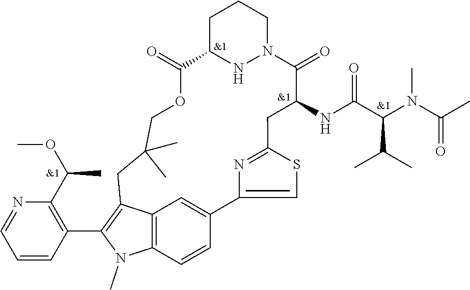 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| A104 | 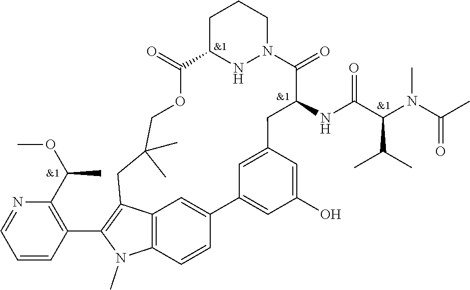 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| A105 | 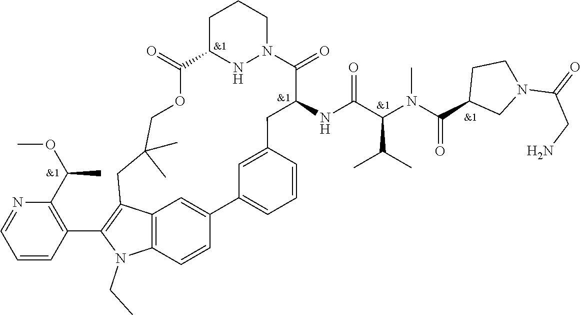 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| A106 | 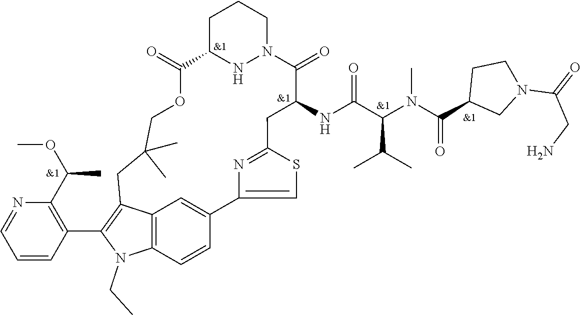 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| A107 | 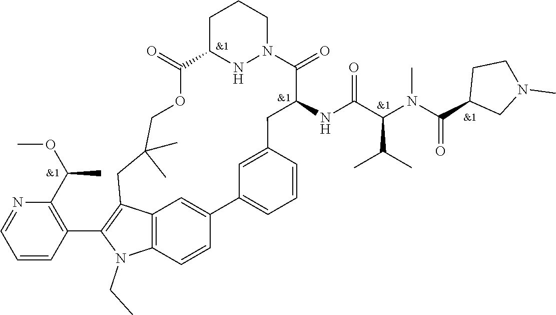 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| A108 | 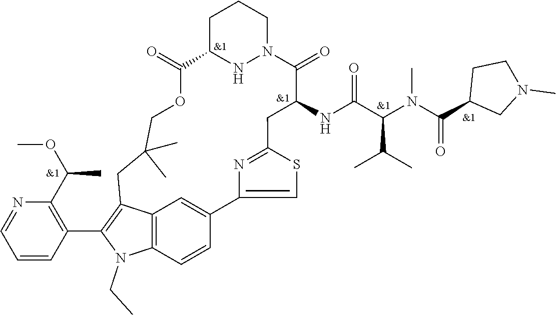 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| A109 | 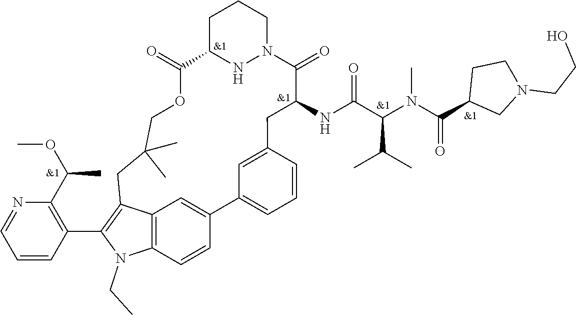 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A110 | 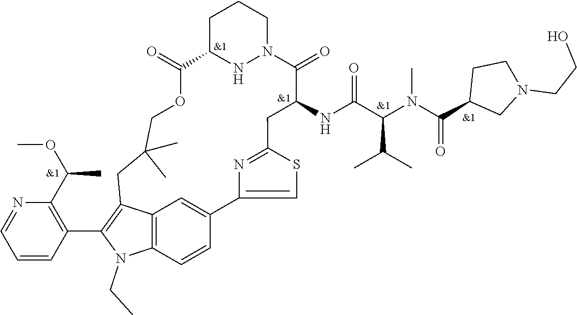 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A111 | 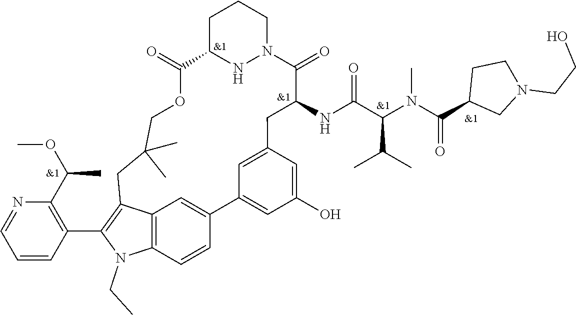 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| A112 | 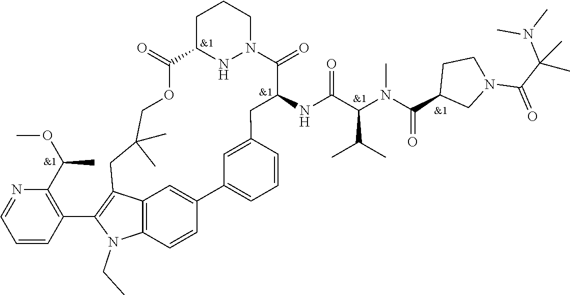 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A113 | 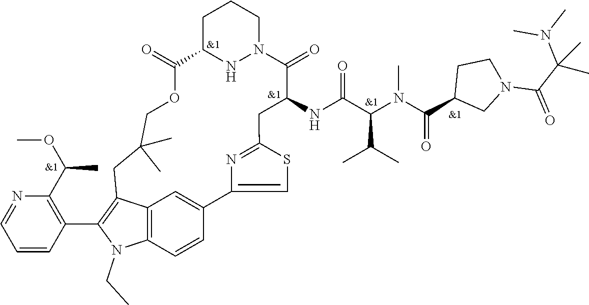 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A114 | 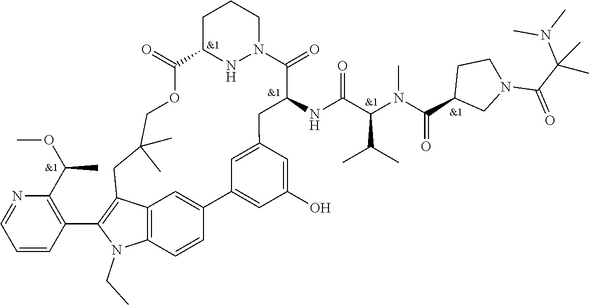 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| A115 | 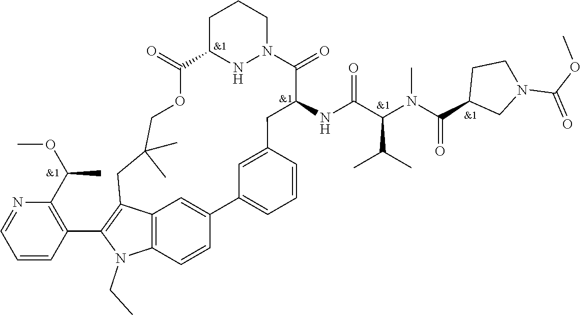 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| A116 | 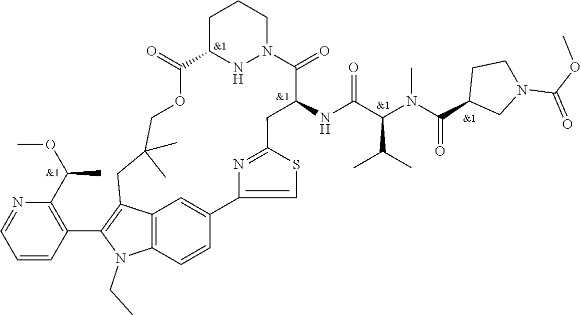 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| A117 | 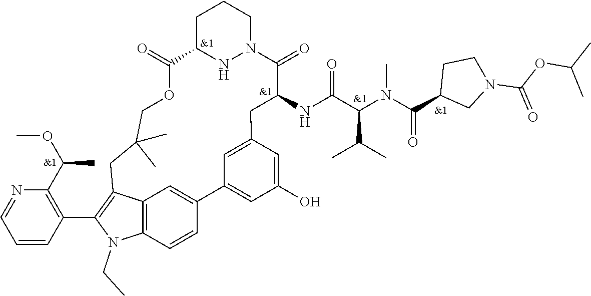 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| A118 | 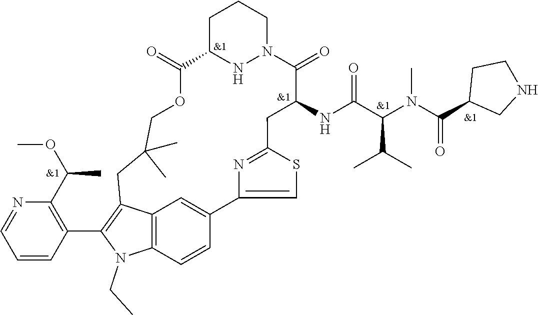 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| A119 | 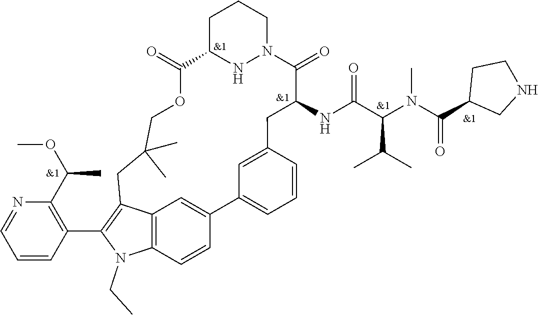 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| A120 | 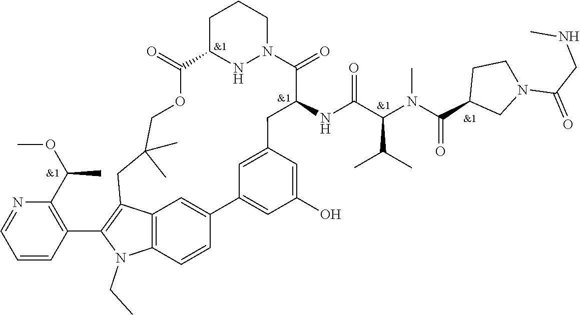 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| A121 | 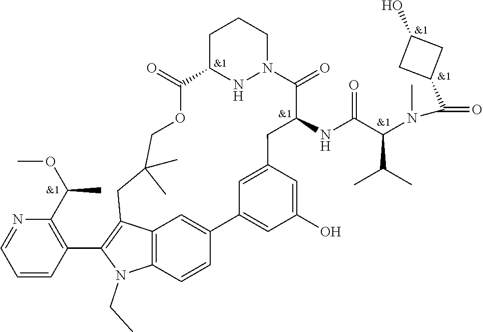 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| A122 | 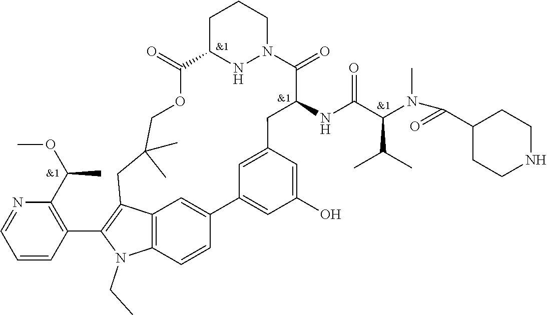 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| A123 | 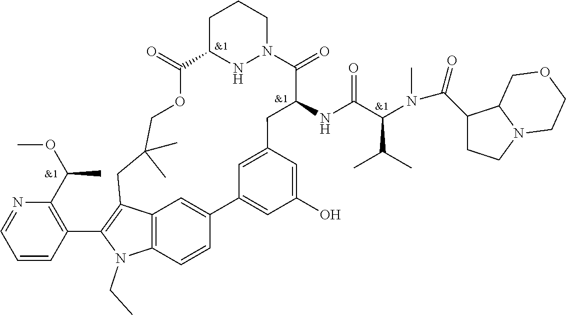 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| A124 | 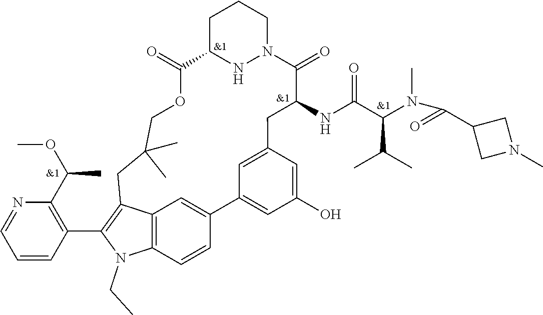 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| A125 | 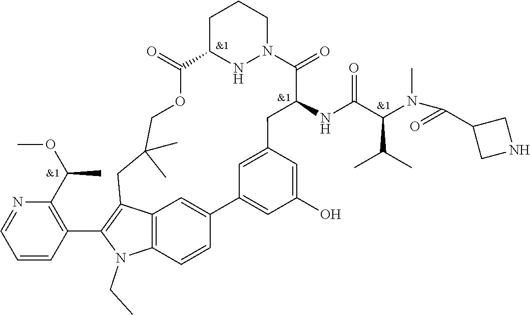 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| A126 | 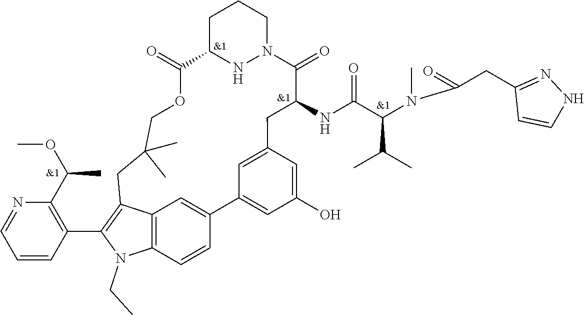 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| A127 | 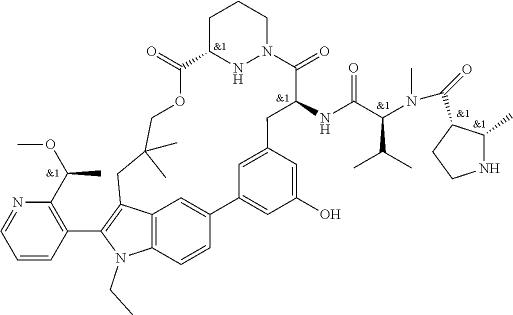 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| A128 | 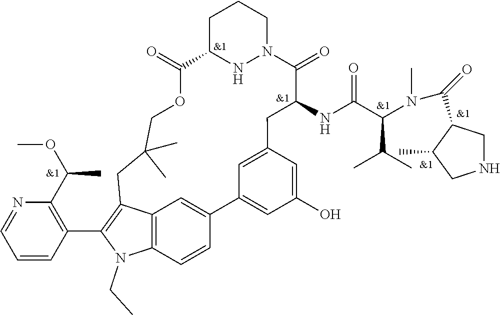 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| A129 | 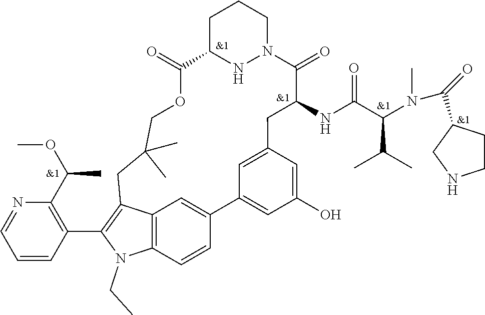 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| A130 | 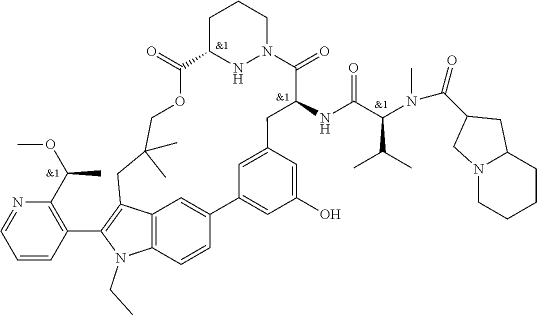 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CCCCN3C1)C(=O)N1CCC[C@H](N1)C(=O)OCC(C)(C)C2] |
| A131 | 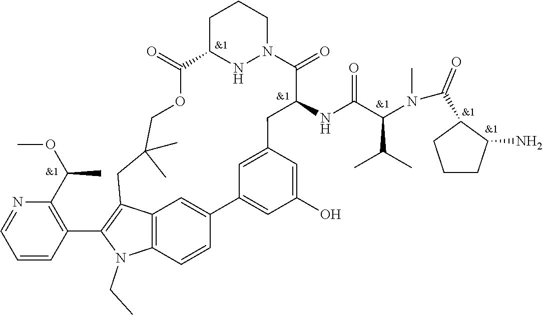 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| A132 | 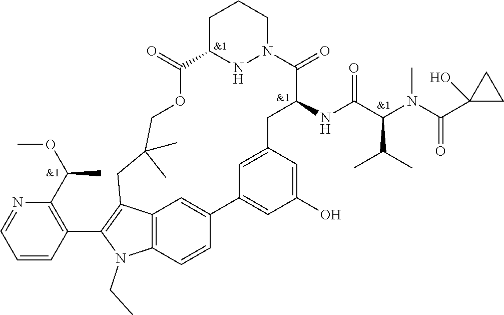 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| A133 | 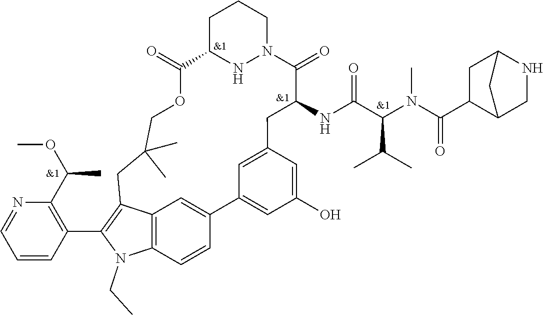 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| A134 | 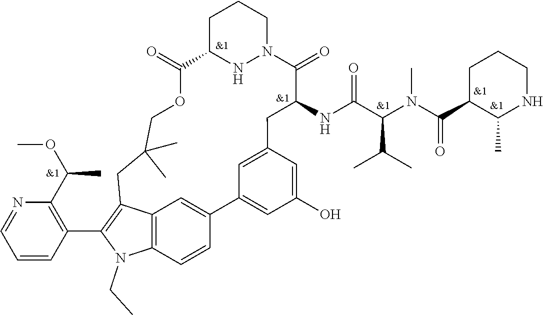 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| A135 | 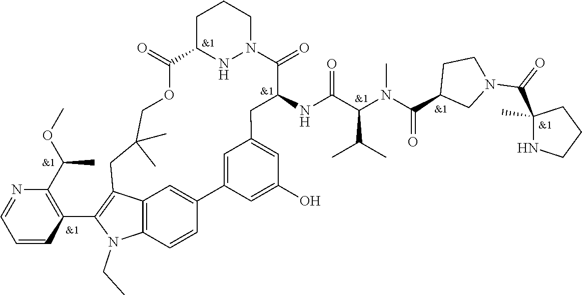 |
| A136 | 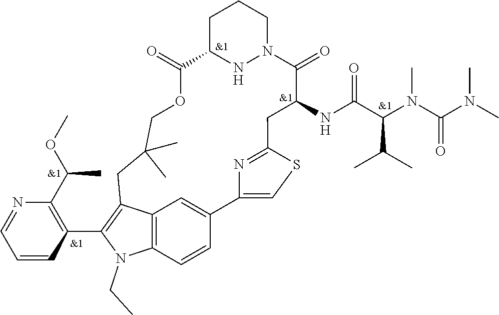 |
| A137 | 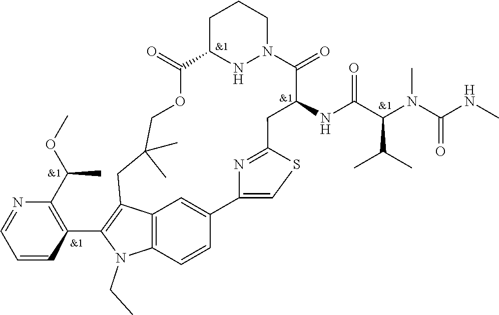 |
| A138 | 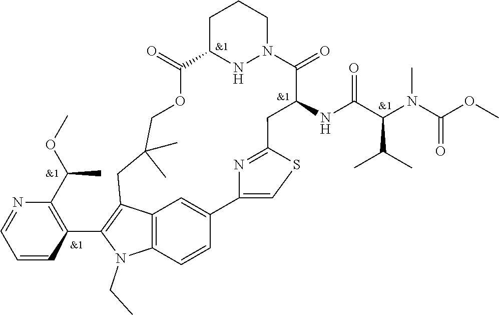 |
| A139 | 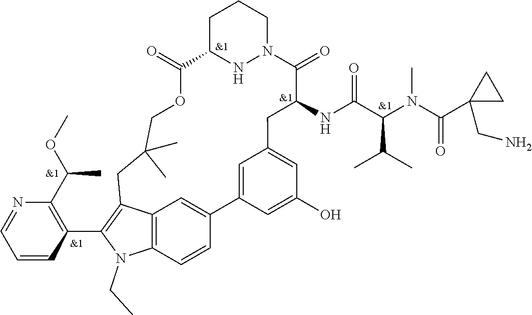 |
| A140 | 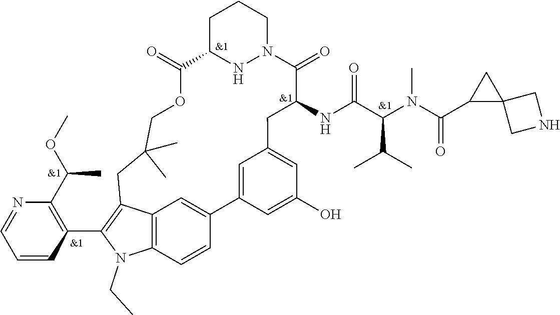 |
| A141 | 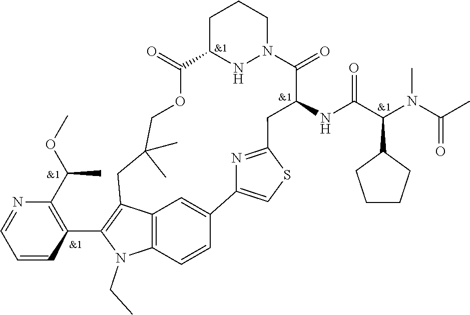 |
| A142 | 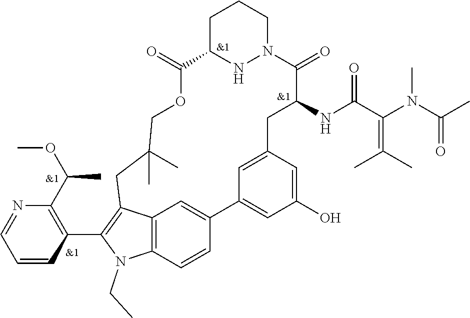 |
| A143 | 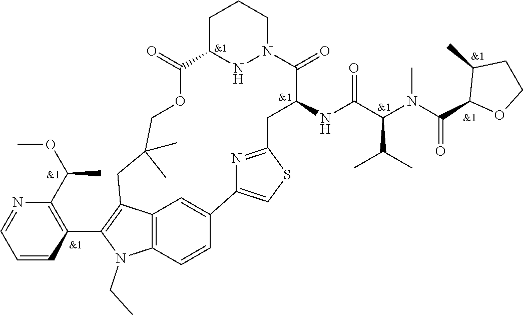 |
| A144 | 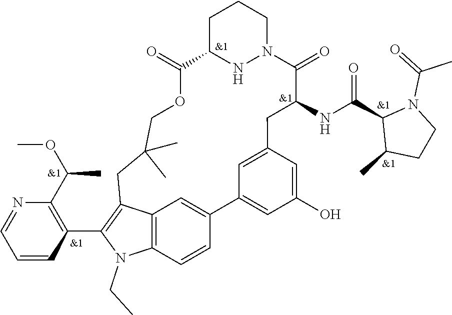 |
| A145 | 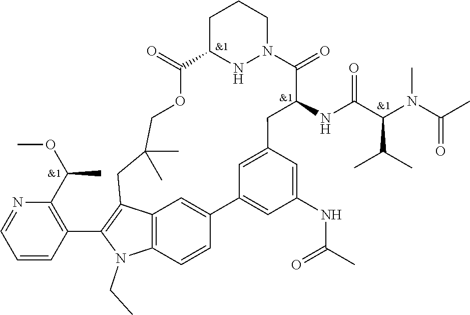 [SMILES: CCn1c(-c2cccnc2[C@H](C)OC)c2c3cc(ccc31)-c1cc(cc(NC(C)=O)c1)C[C@H](NC(=O)[C@H](C(C)C)N(C)C(C)=O)C(=O)N1CCC[C@H](N1)C(=O)OCC(C)(C)C2] |
| A146 | 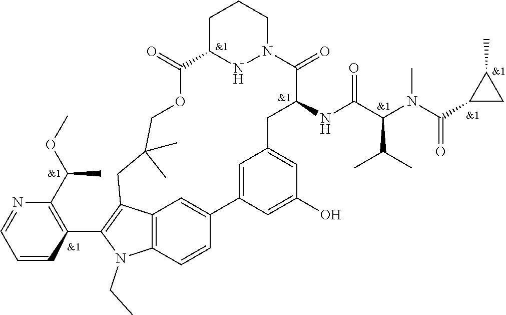 |
| A147 | 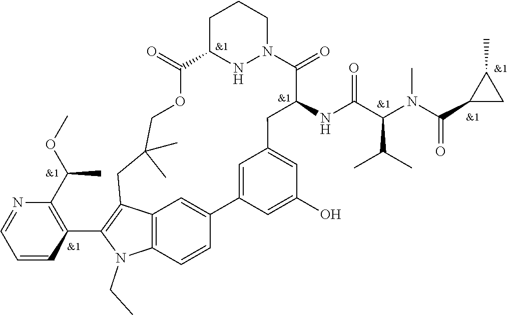 |
| A148 | 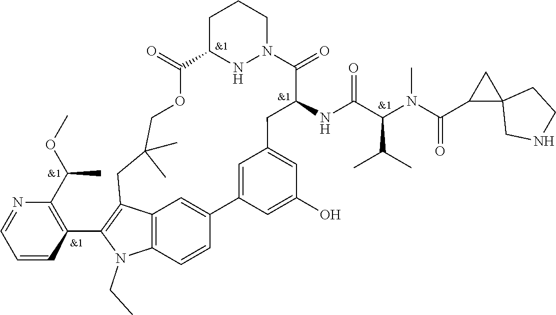 |
| A149 | 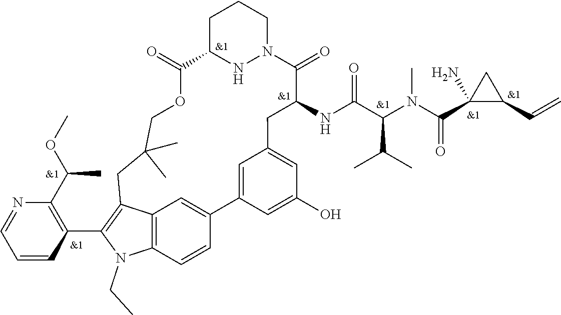 |
| A150 | 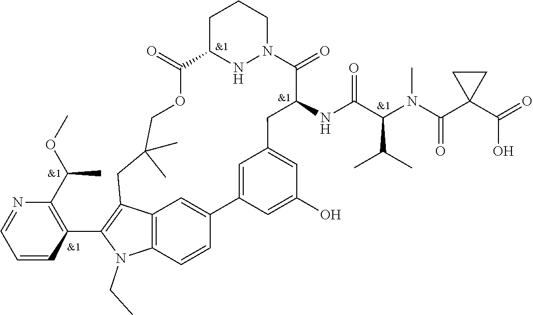 |
| A151 | 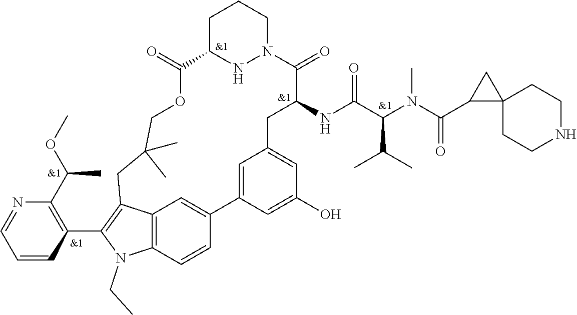 |
| A152 | 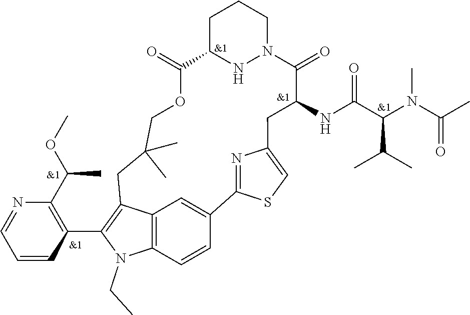 |
| A153 | 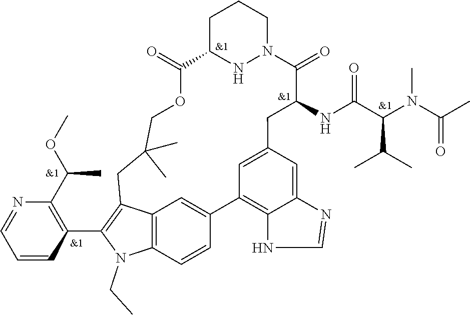 |
| A154 | 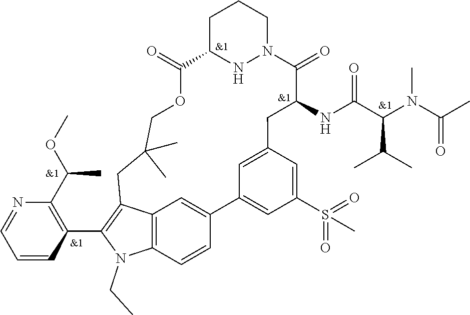 |
| A155 | 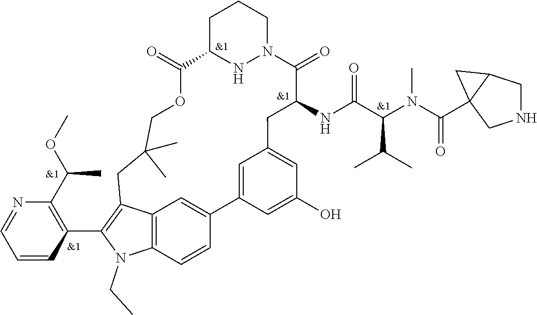 |
| A156 | 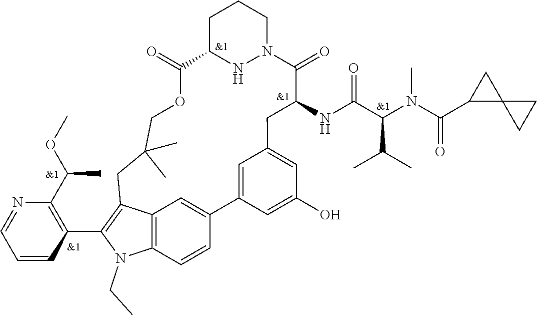 |
| A157 | 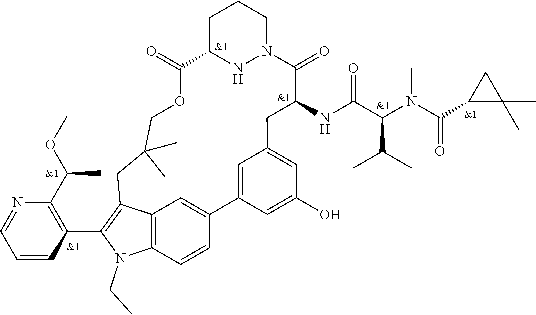 |
| A158 | 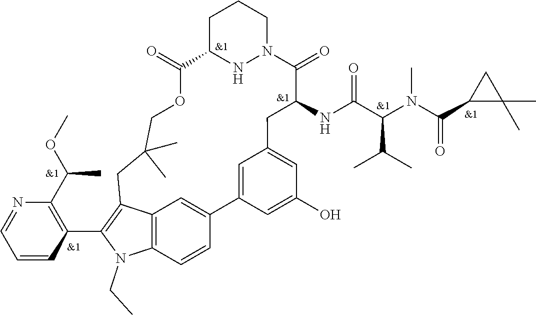 |
| A159 | 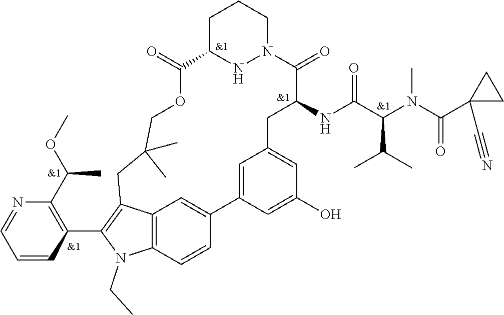 |
| A160 | 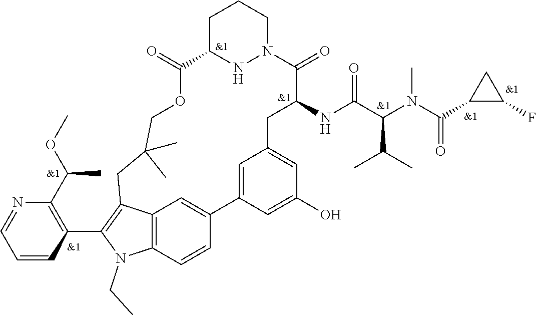 |
| A161 | 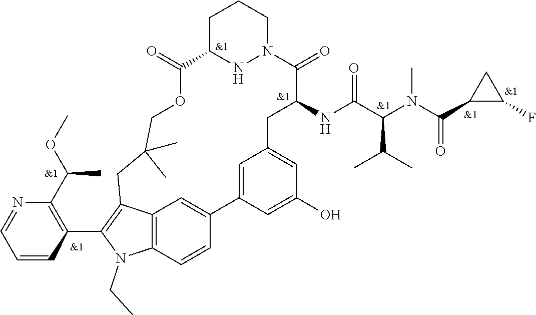 |
| A162 | 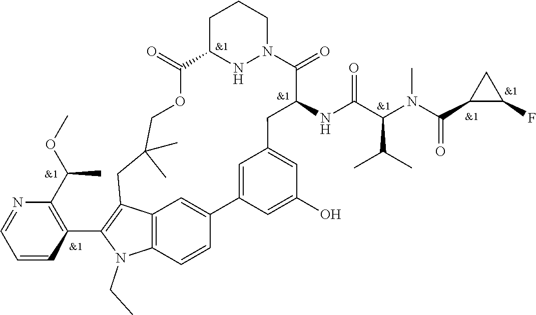 |
| A163 | 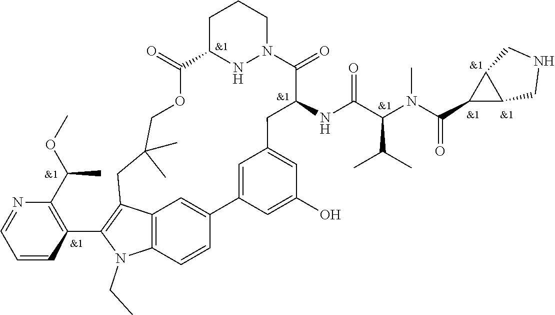 |
| A164 | 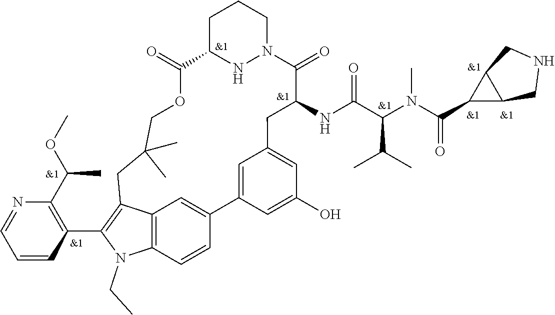 |
| A165 | 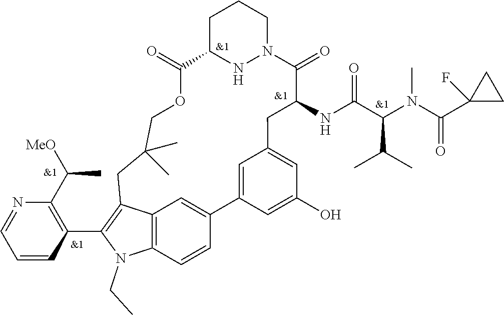 |
| A166 | 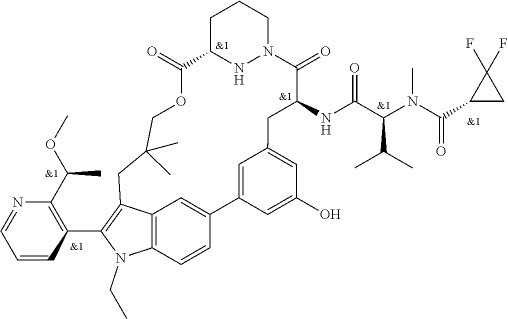 |
| A167 | 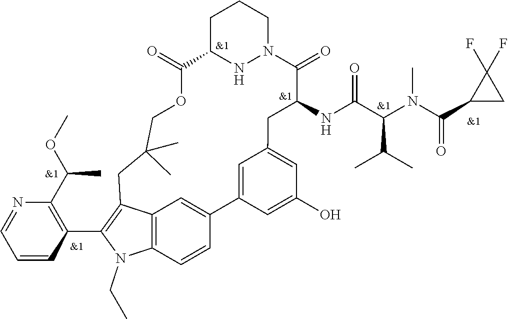 |
| A168 | 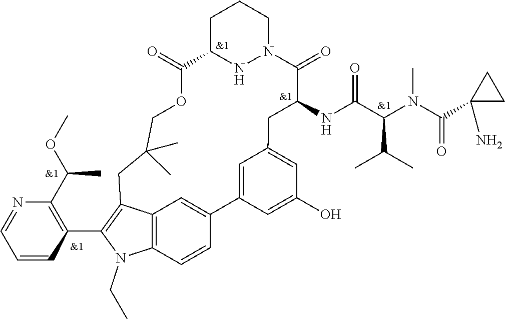 |
| A169 | 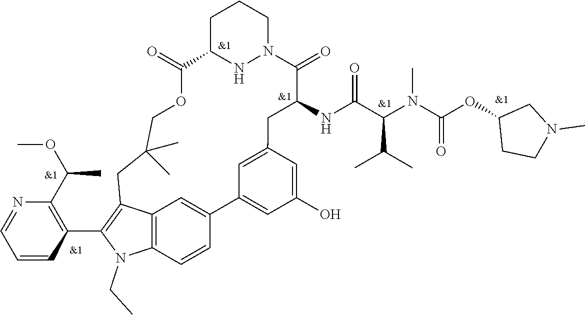 |
| A170 | 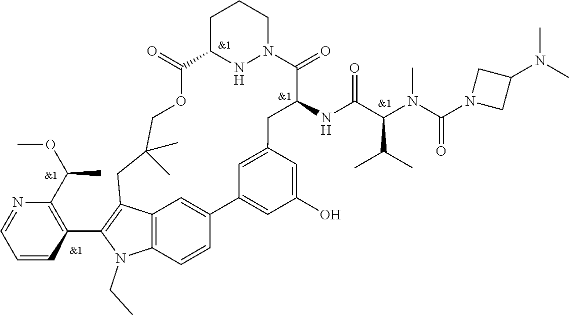 |
| A171 | 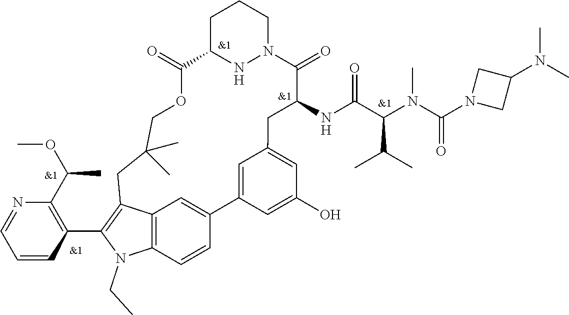 |
| A172 | 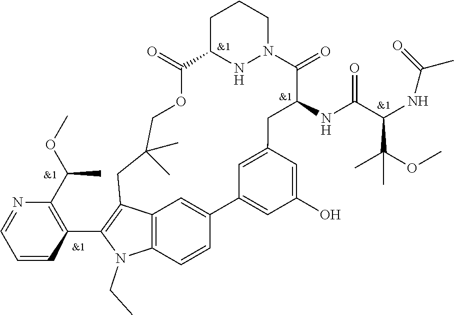 |
| A173 | 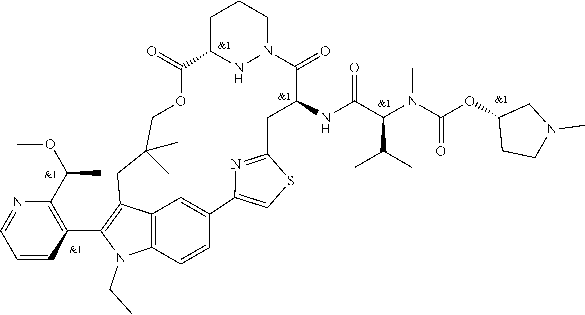 |
| A174 | 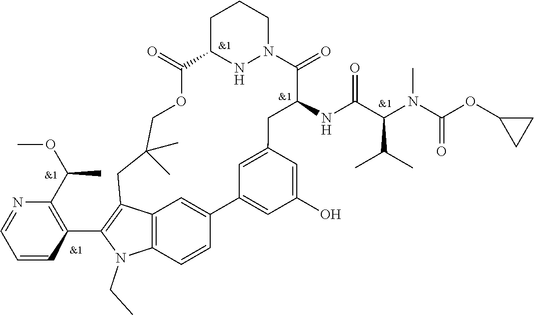 |
| A175 | 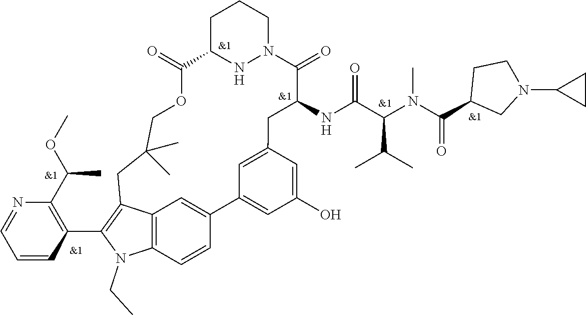 |
| A176 | 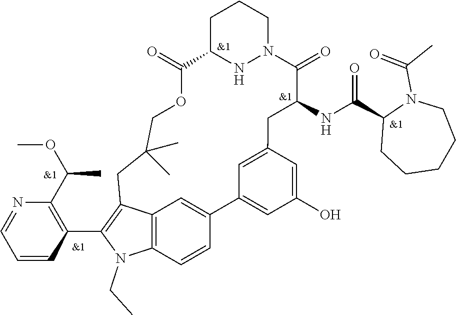 |
| A177 | 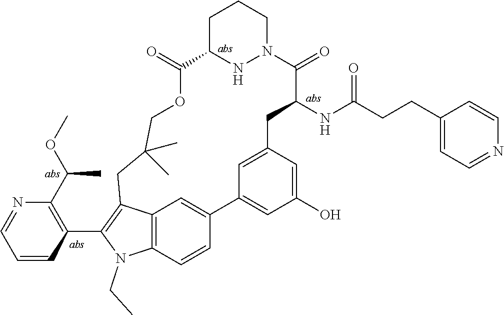 |
| A178 | 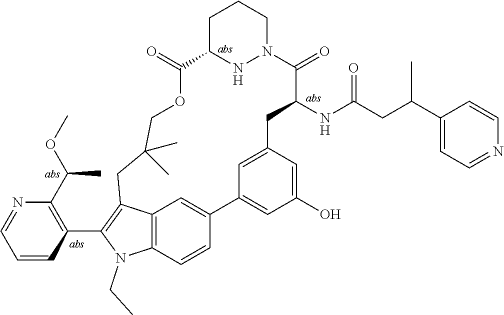 |
| A179 | 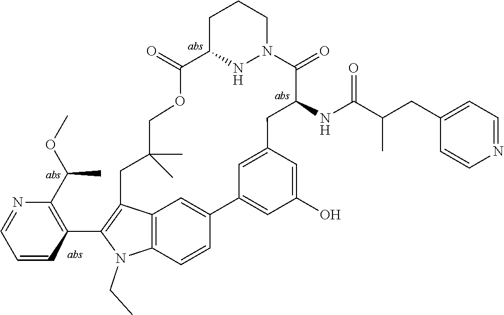 |
| A180 | 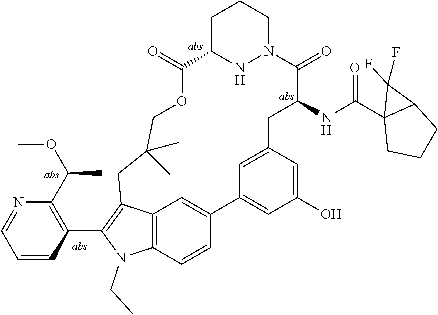 |
| A181 | 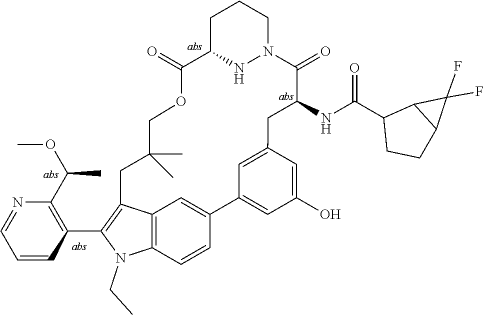 |
| A182 | 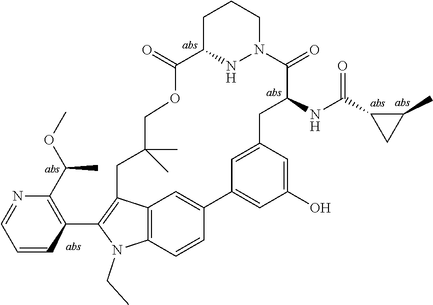 |
| A183 | 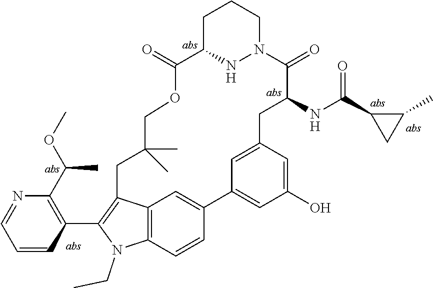 |
| A184 | 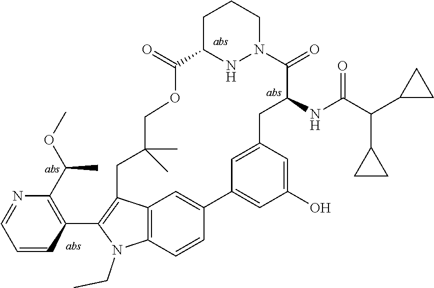 |
| A185 | 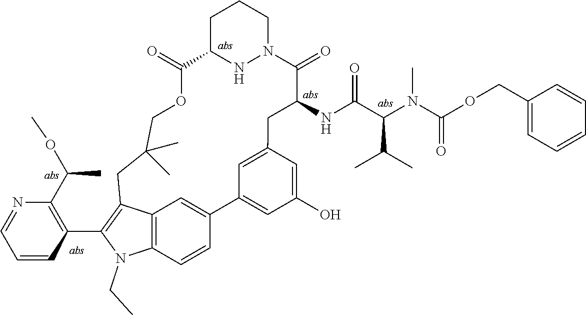 |
| A186 | 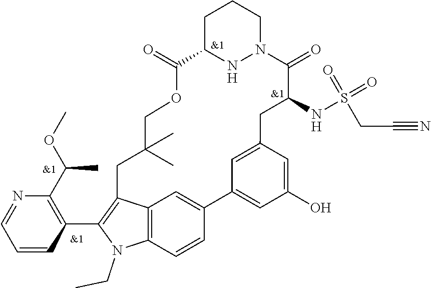 |
| A187 | 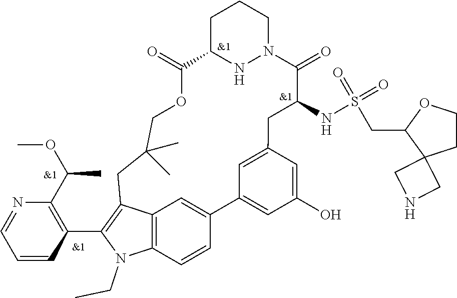 |
| A188 | 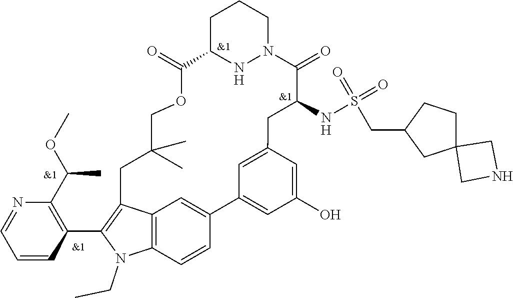 |
| A189 | 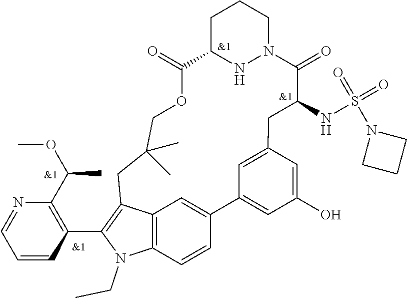 |
| A190 | 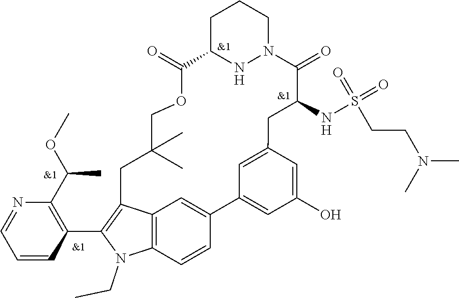 |
| A191 | 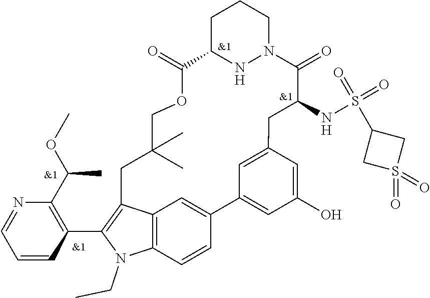 |
| A192 | 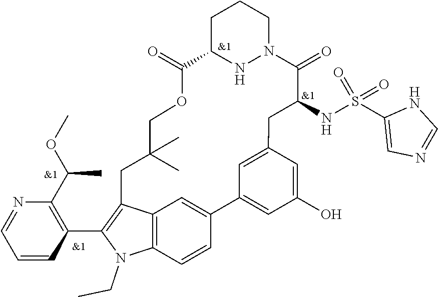 |
| A193 | 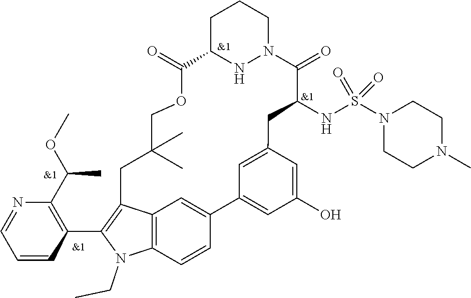 |
| A194 | 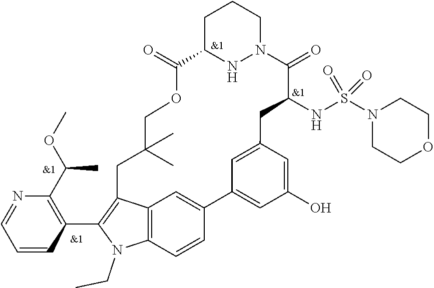 |
| A195 | 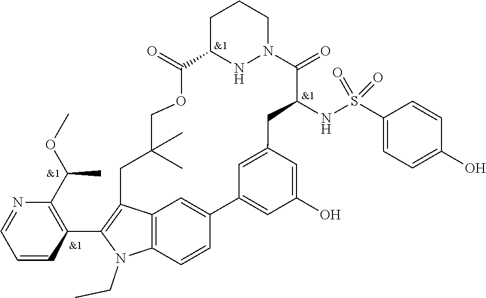 |
| A196 | 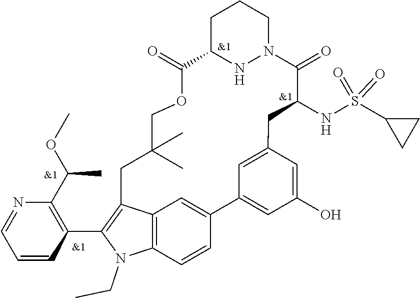 |
| A197 | 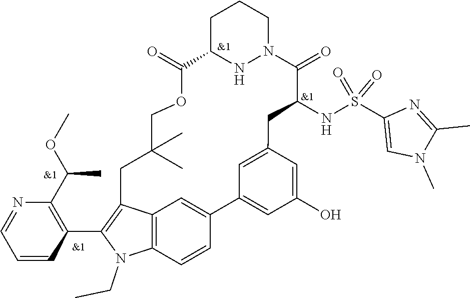 |
| A198 | 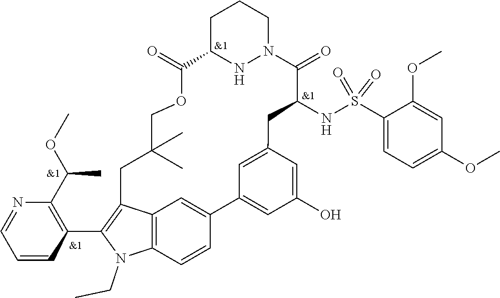 |
| A199 | 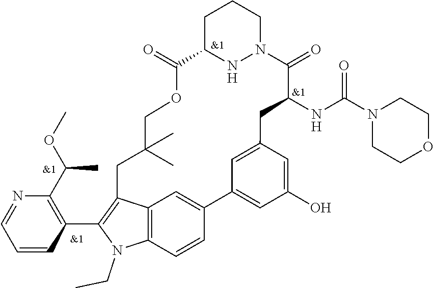 |
| A200 | 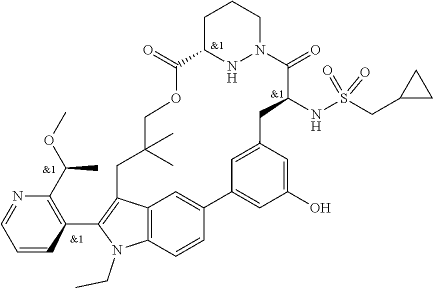 |
| A201 | 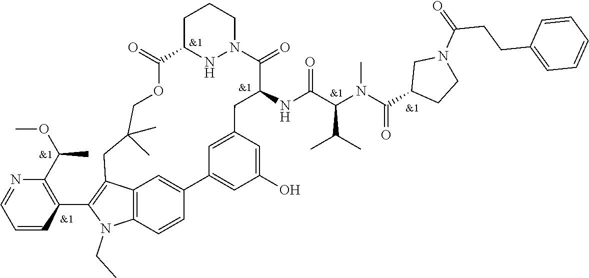 |
| A202 | 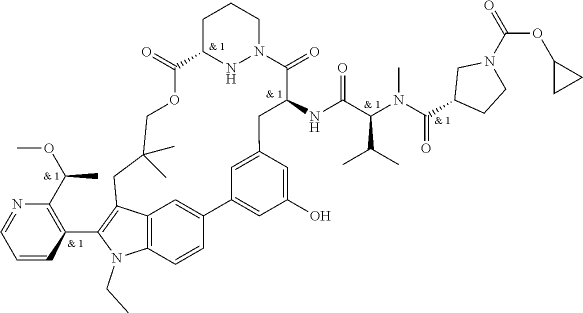 |
| A203 | 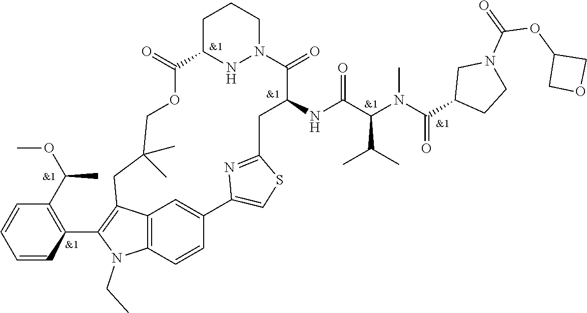 |
| A204 | 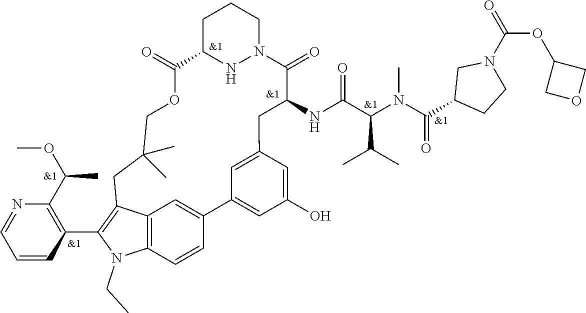 |
| A205 | 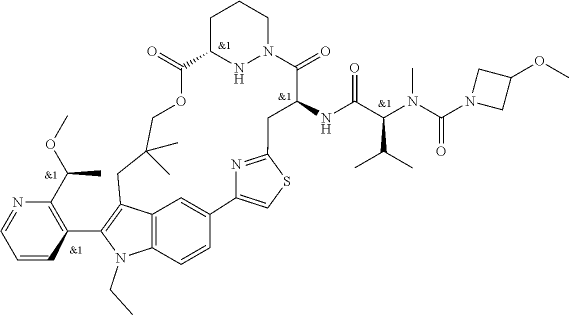 |
| A206 | 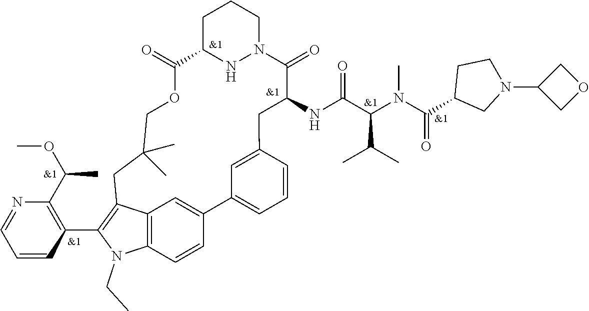 |
| A207 | 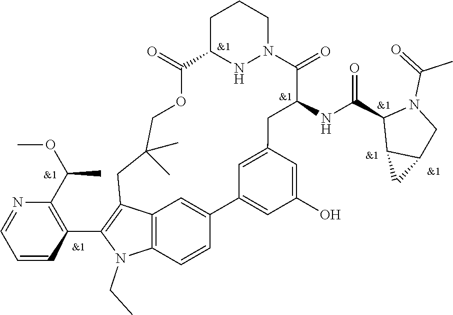 |
| A208 | 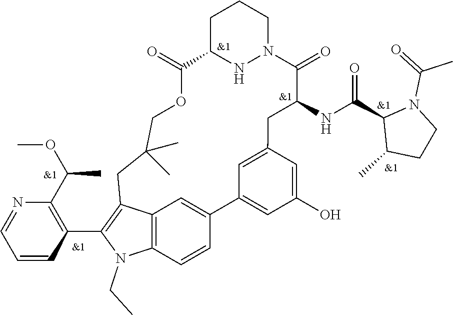 |
| A209 | 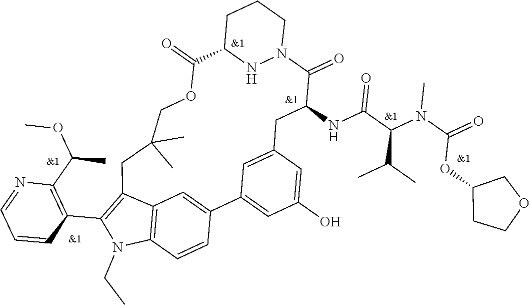 |
| A210 | 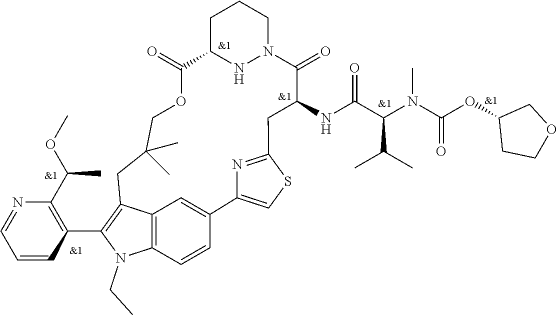 |
| A211 | 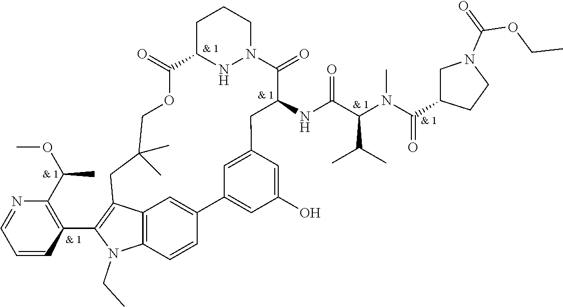 |
| A212 | 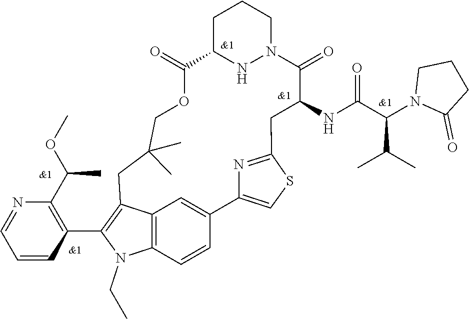 |
| A213 | 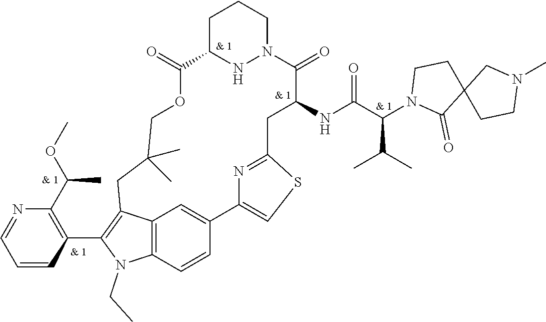 |
| A214 | 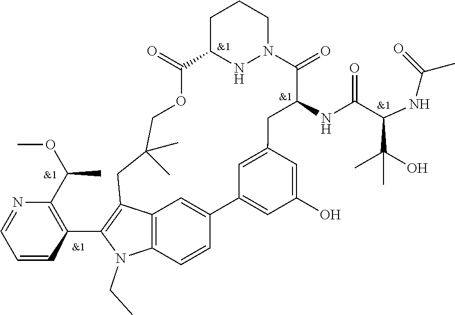 |
| A215 | 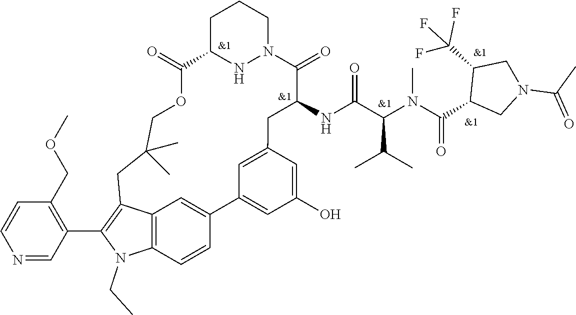 [SMILES: CCn1c(-c2cnccc2COC)c2c3cc(ccc31)-c1cc(O)cc(c1)C[C@H](NC(=O)[C@H](C(C)C)N(C)C(=O)[C@H]1CN(C(C)=O)C[C@H]1C(F)(F)F)C(=O)N1CCC[C@H](N1)C(=O)OCC(C)(C)C2] |
| A216 | 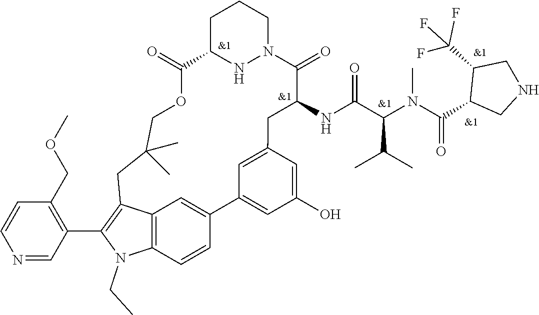 [SMILES: CCn1c(-c2cnccc2COC)c2c3cc(ccc31)-c1cc(O)cc(c1)C[C@H](NC(=O)[C@H](C(C)C)N(C)C(=O)[C@H]1CNC[C@H]1C(F)(F)F)C(=O)N1CCC[C@H](N1)C(=O)OCC(C)(C)C2] |
| A217 | 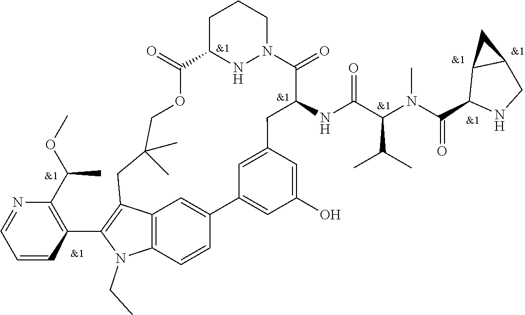 |
| A218 | 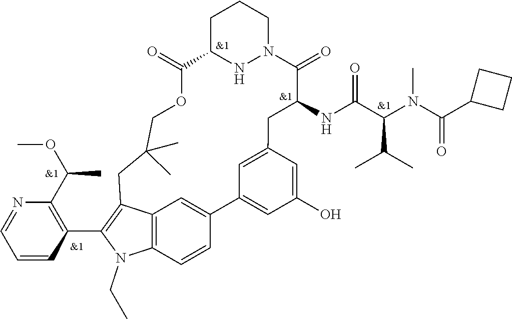 |
| A219 | 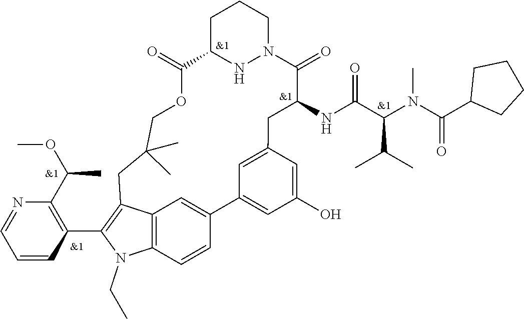 |
| A220 | 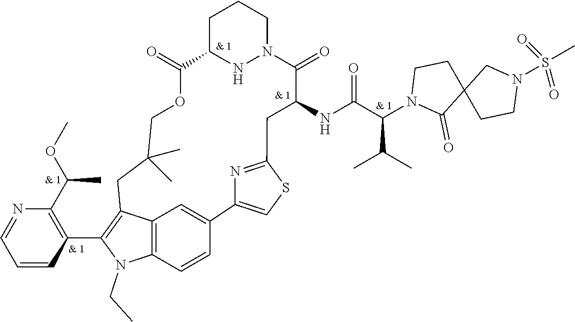 |
| A221 | 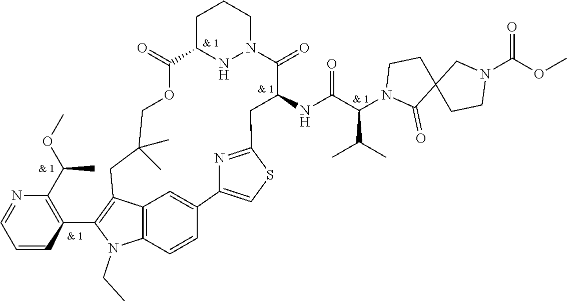 |
| A222 | 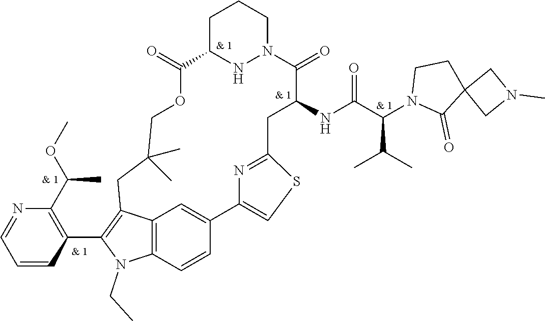 |
| A223 | 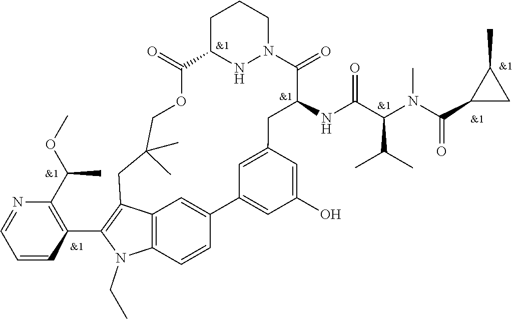 |
| A224 | 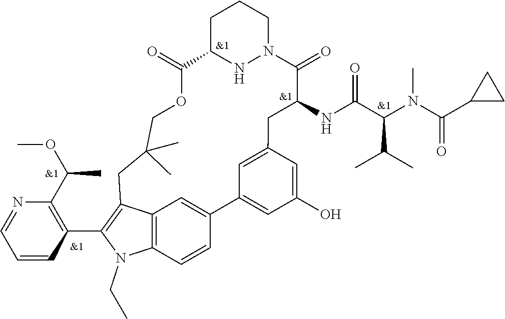 |
| A225 | 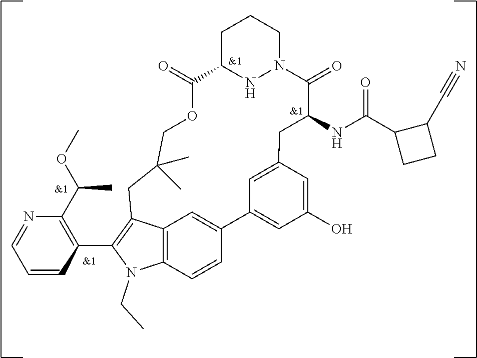 |
| A226 | 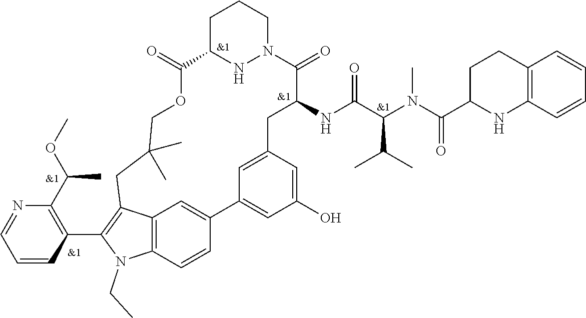 |
| A227 | 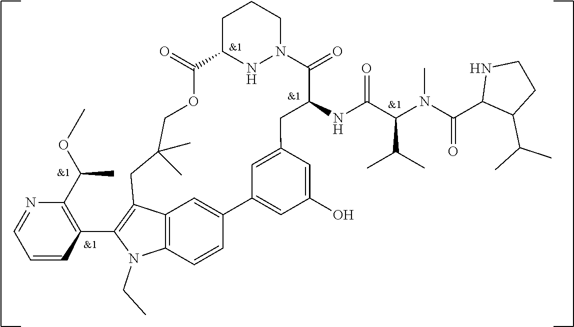 |
| A228 | 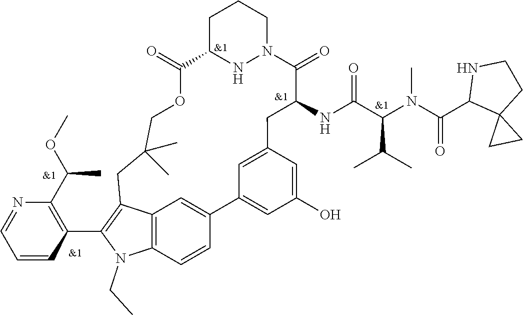 |
| A229 | 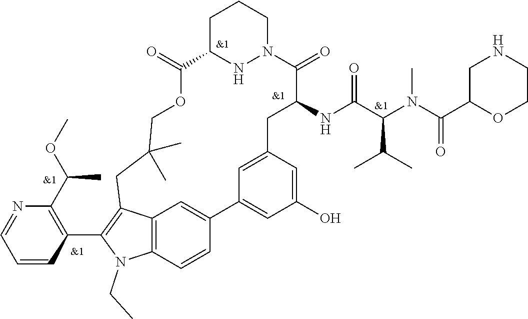 |
| A230 | 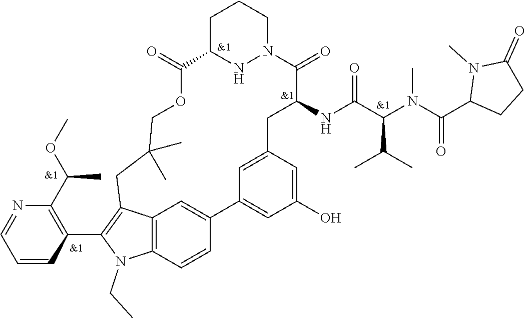 |
| A231 | 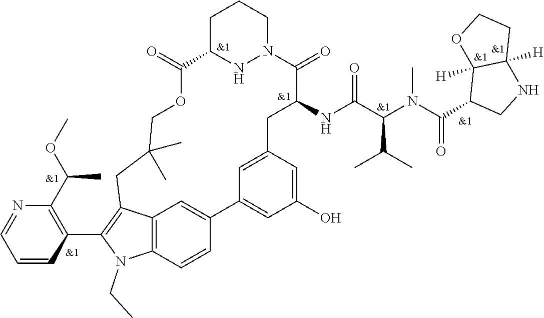 |
| A232 | 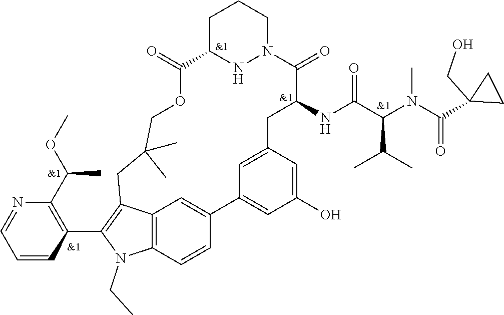 |
| A233 | 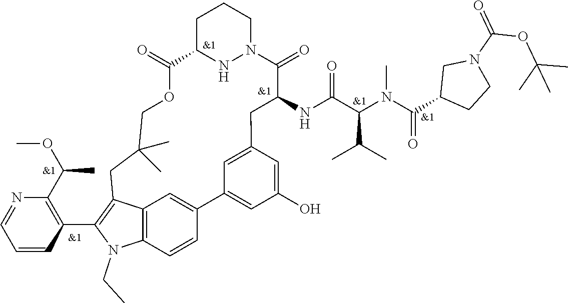 |
| A234 | 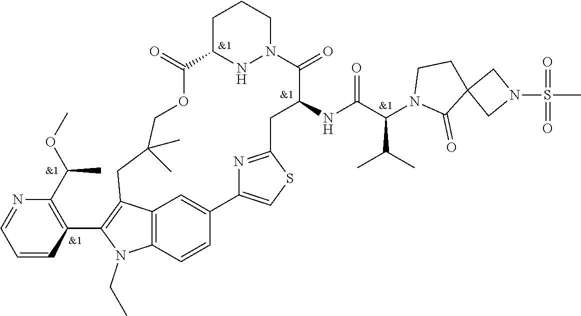 |
| A235 | 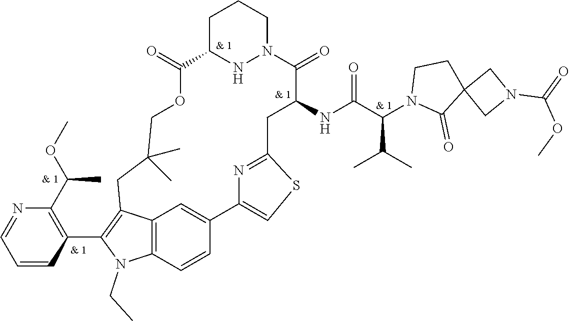 |
| A236 | 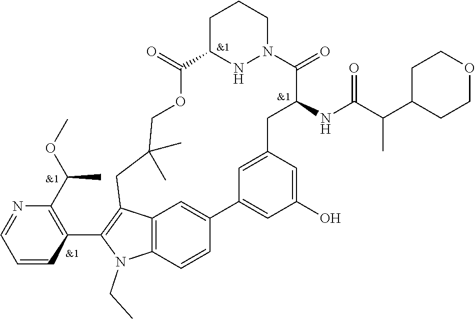 |
| A237 | 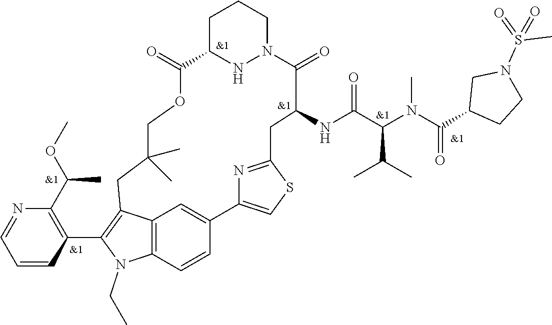 |
| A238 | 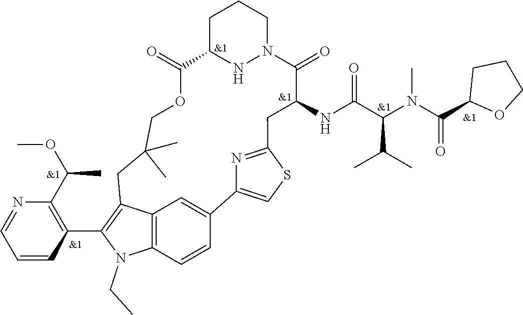 |
| A239 | 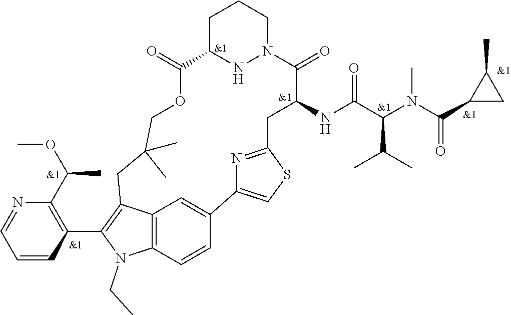 |
| A240 | 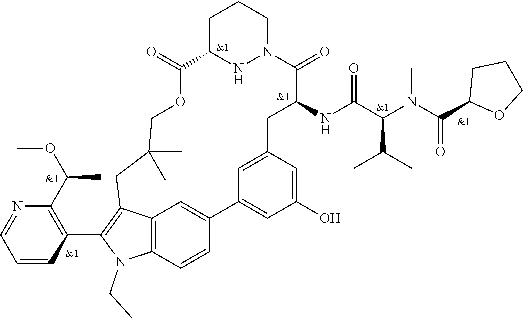 |
| A241 | 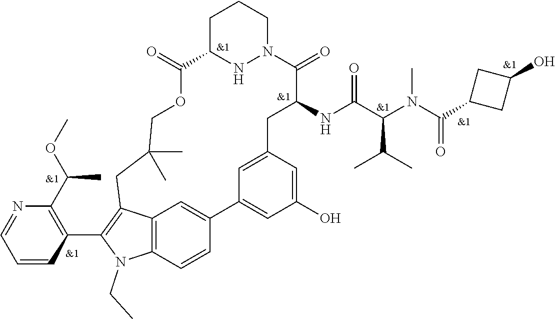 |
| A242 | 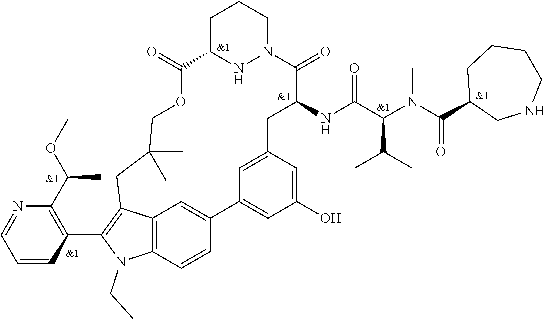 |
| A243 | 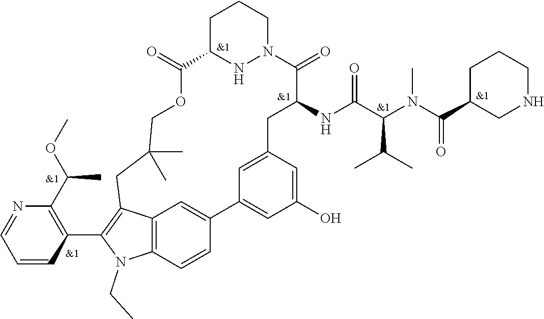 |
| A244 | 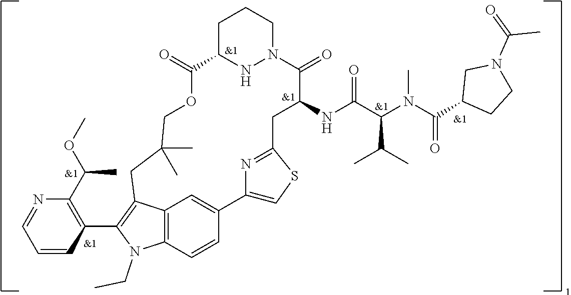 |
| A245 | 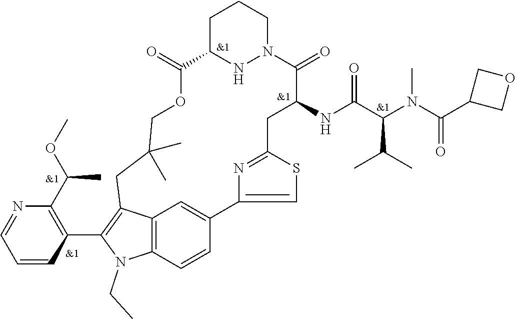 |
| A246 | 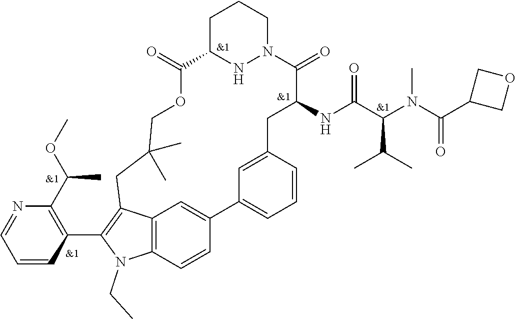 |
| A247 | 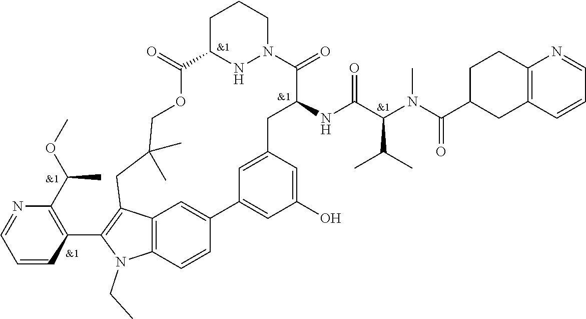 |
| A248 | 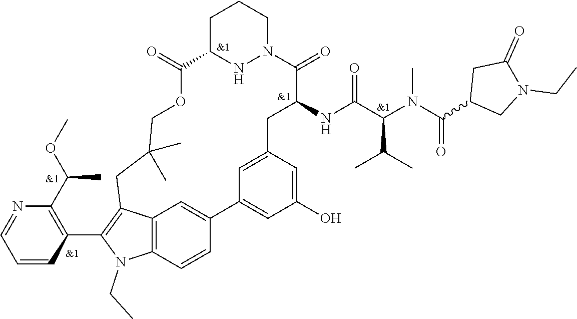 |
| A249 | 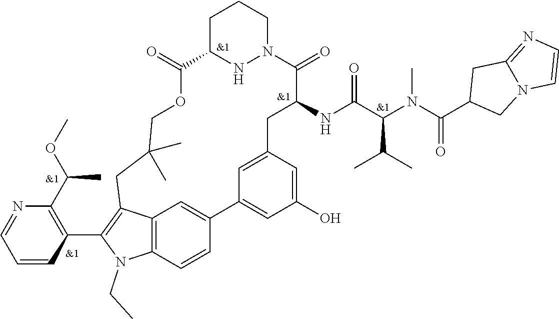 |
| A250 | 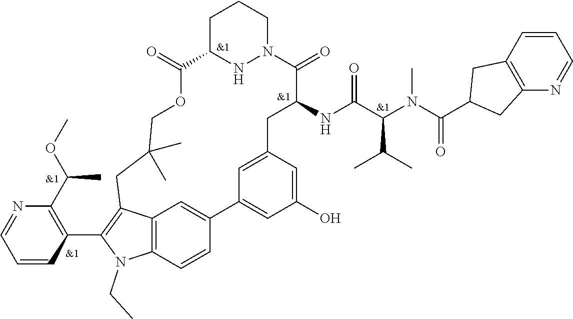 |
| A251 | 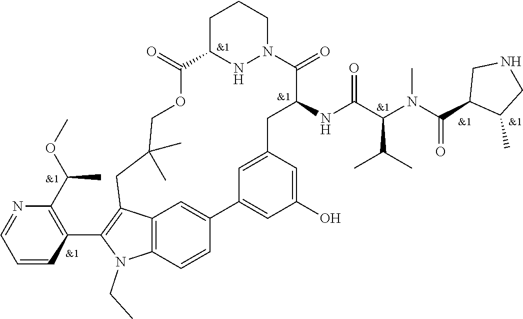 |
| A252 | 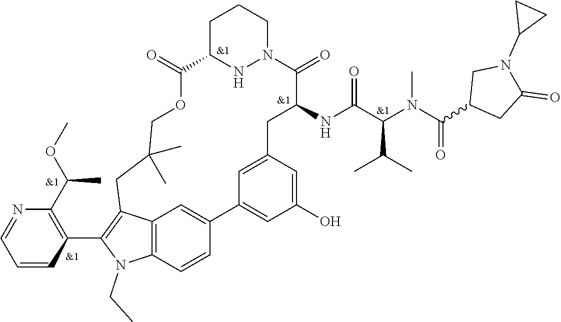 |
| A253 | 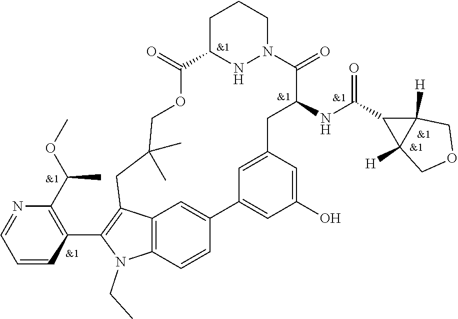 |
| A254 | 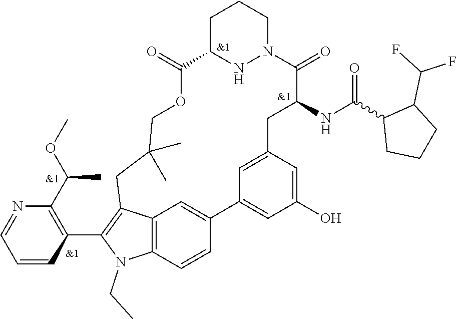 |
| A255 | 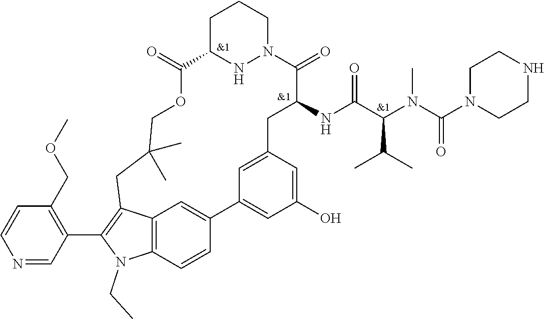 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| A256 | 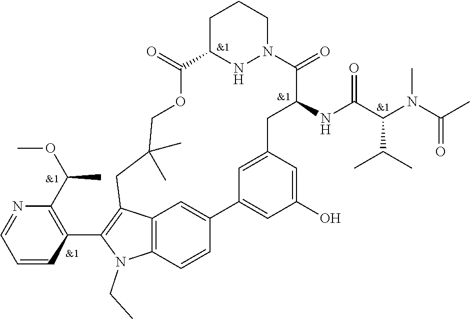 |
| A257 | 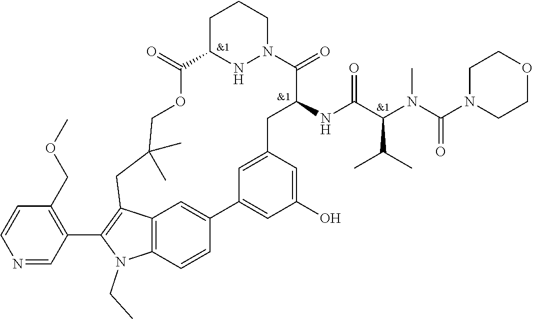 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| A258 | 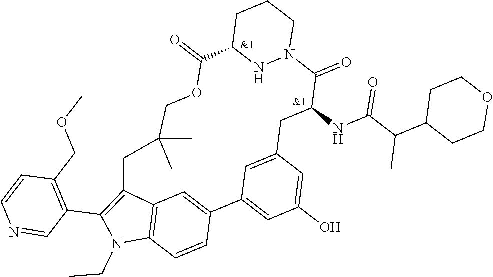 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| A259 | 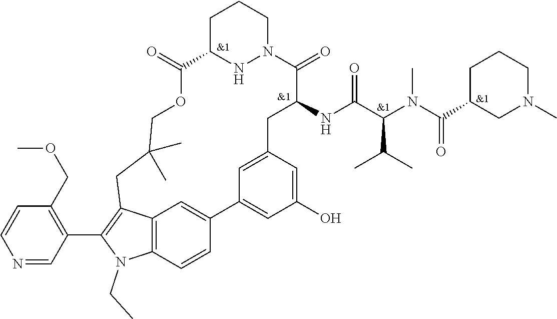 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| A260 | 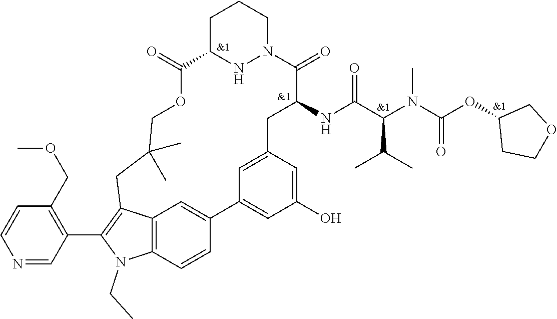 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| A261 | 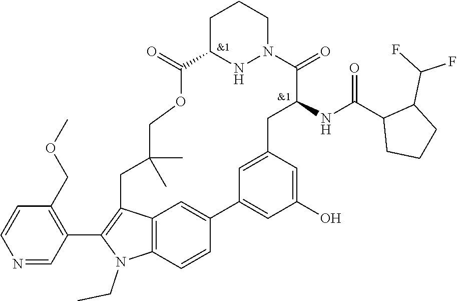 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| A262 | 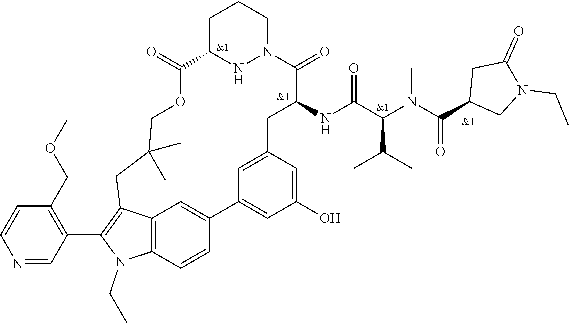 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| A263 | 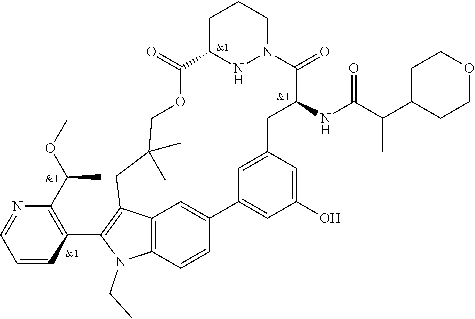 |
| A264 | 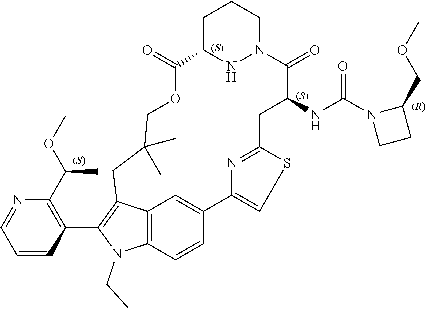 |
| A265 | 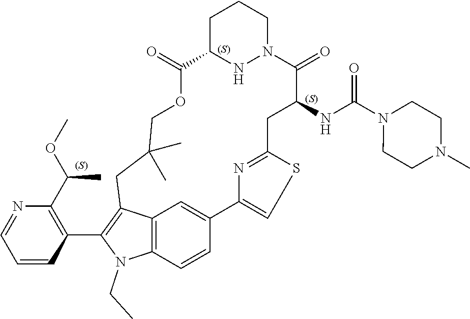 |
| A266 | 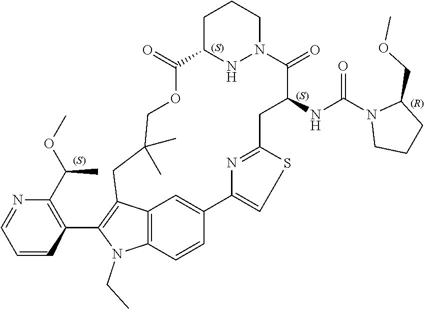 |
| A267 | 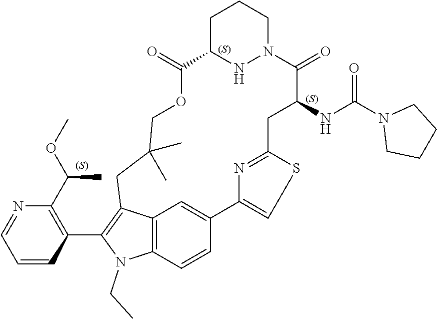 |
| A268 | 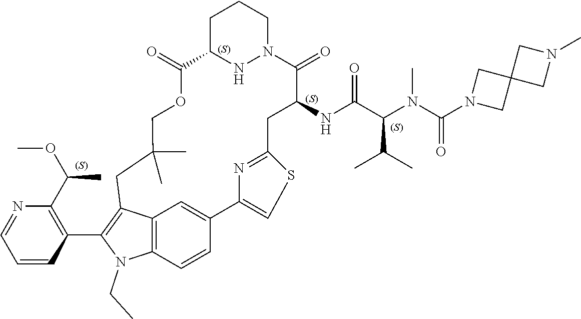 |
| A270 | 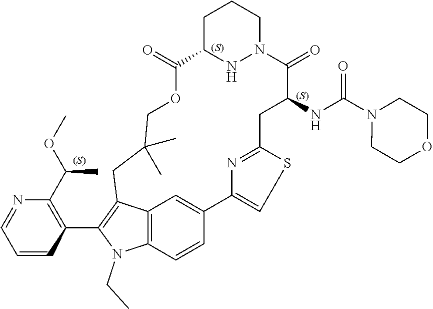 |
| A271 | 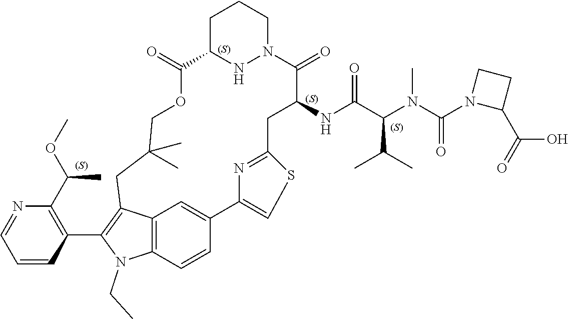 |
| A272 | 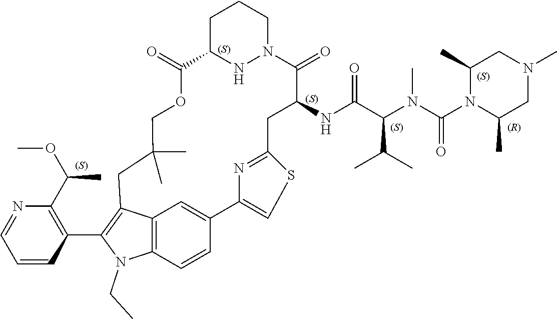 |
| A273 | 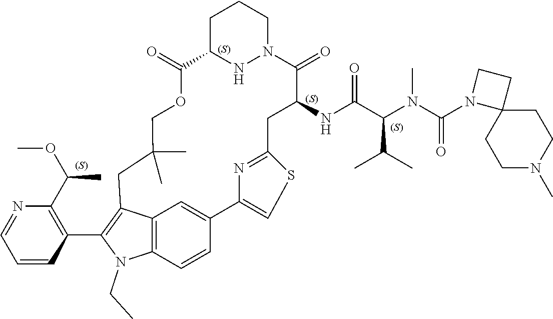 |
| A274 | 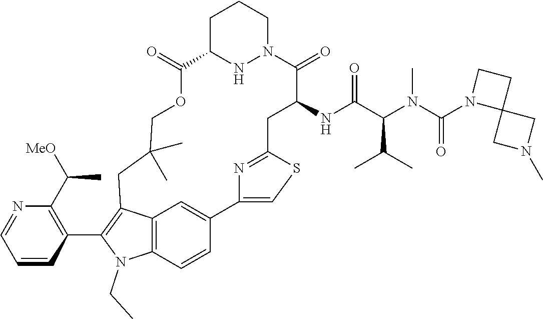 |
| A275 | 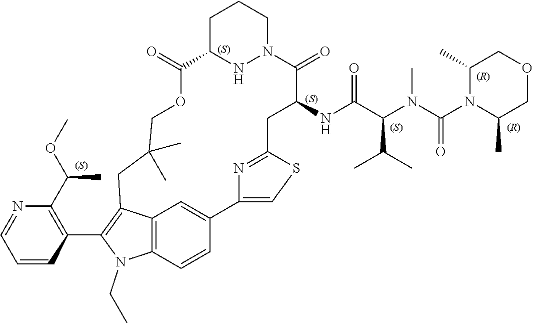 |
| A276 | 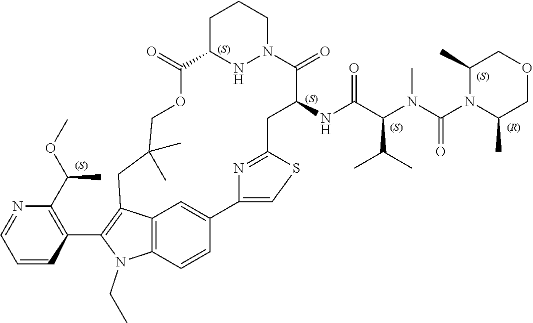 |
| A277 | 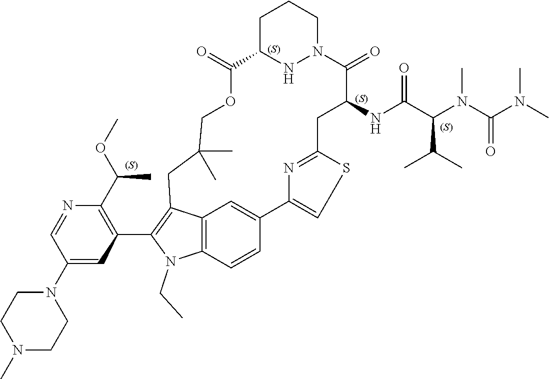 |
| A278 | 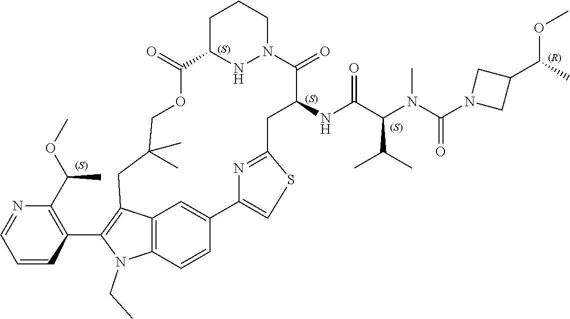 |
| A279 | 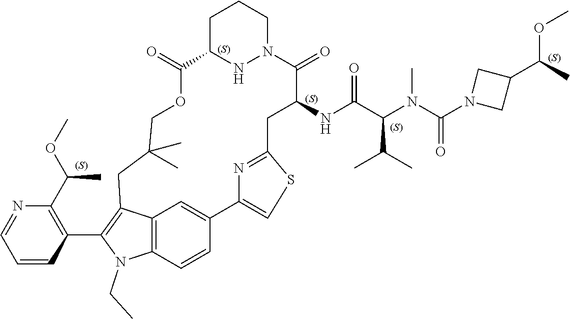 |
| A280 | 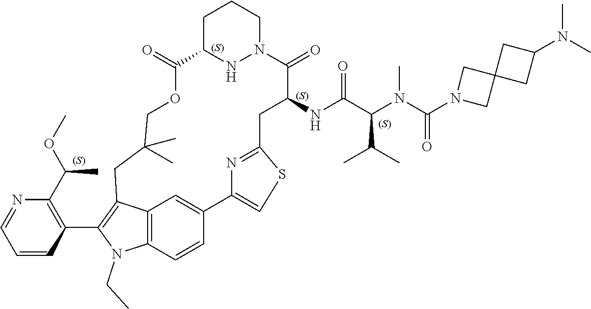 |
| A281 | 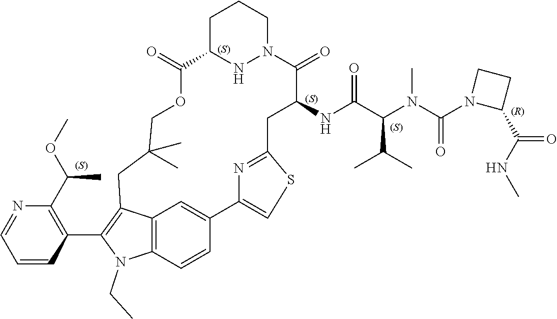 |
| A282 | 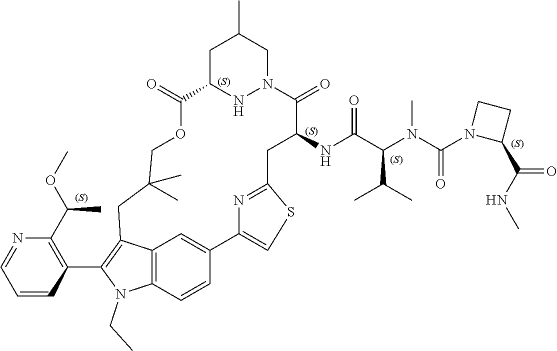 |
| A283 | 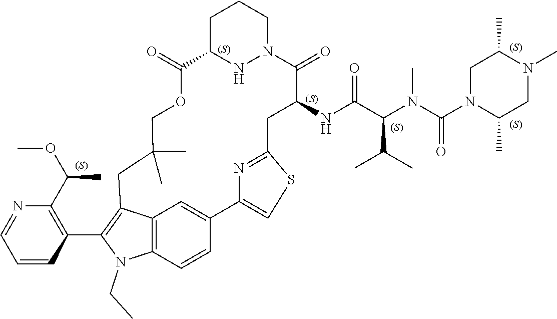 |
| A284 | 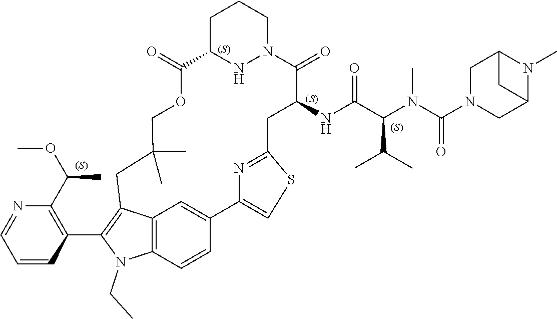 |
| A285 | 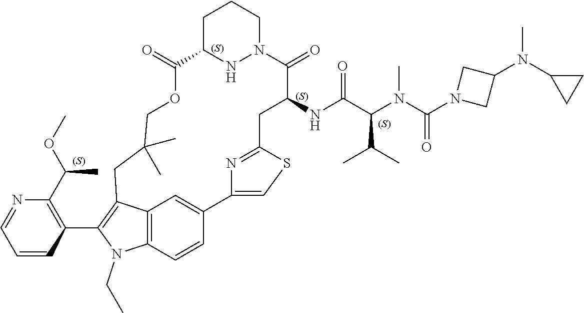 |
| A286 | 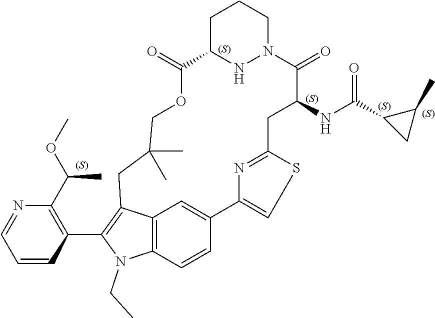 |
| A287 | 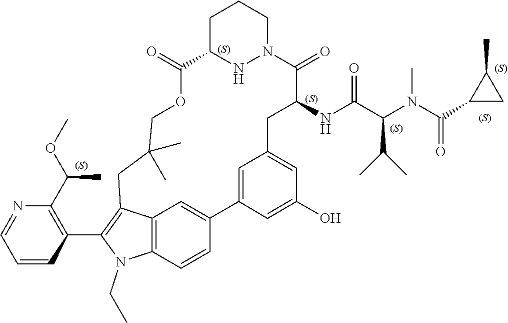 |
| A288 | 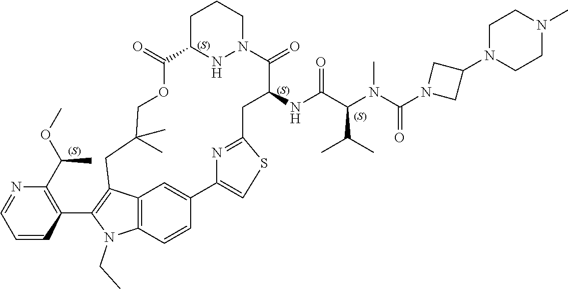 |
| A289 | 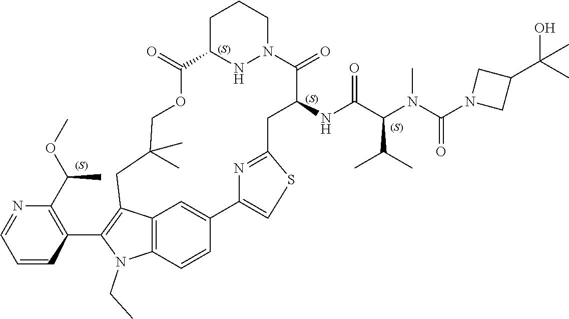 |
| A290 | 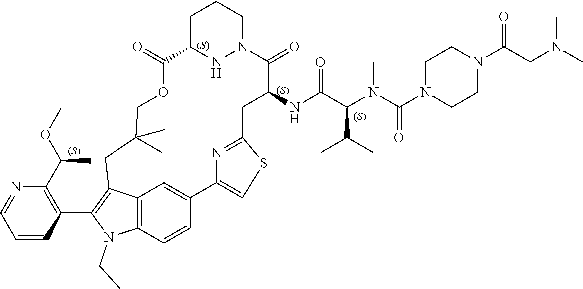 |
| A291 | 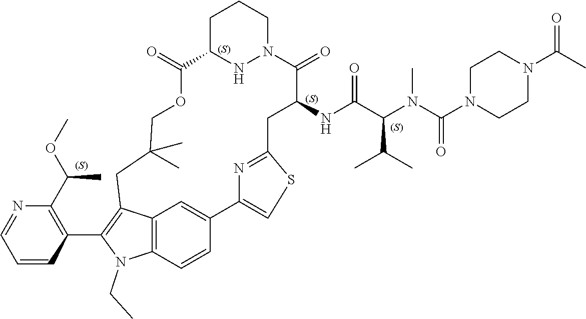 |
| A292 | 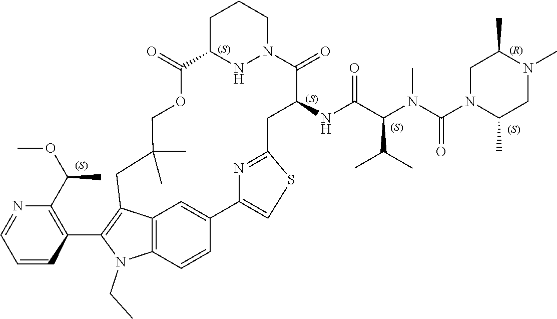 |
| A293 | 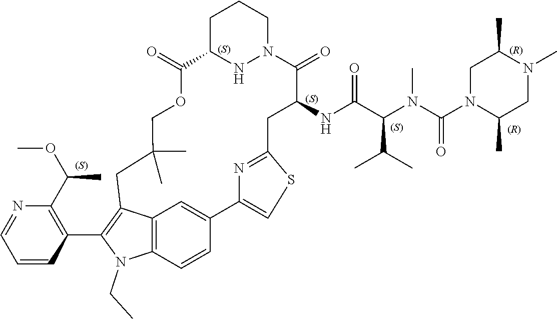 |
| A294 | 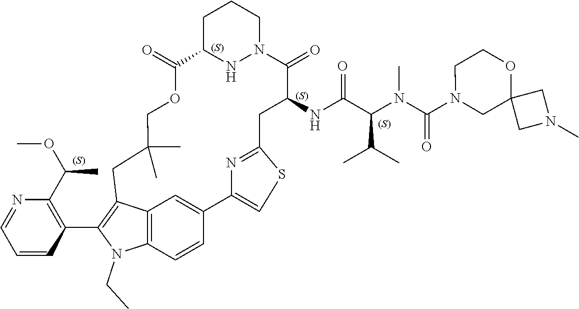 |
| A295 | 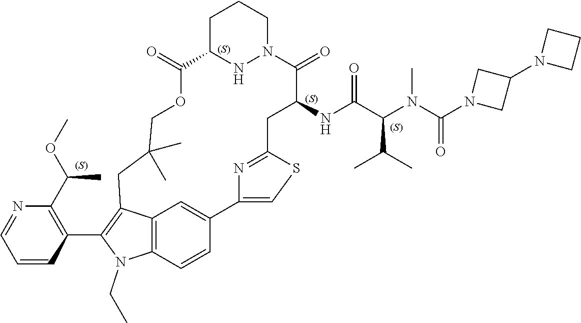 |
| A296 | 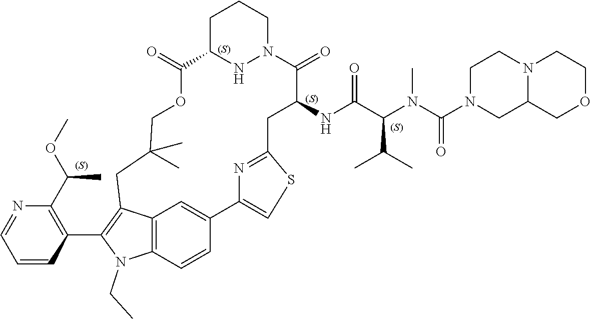 |
| A297 | 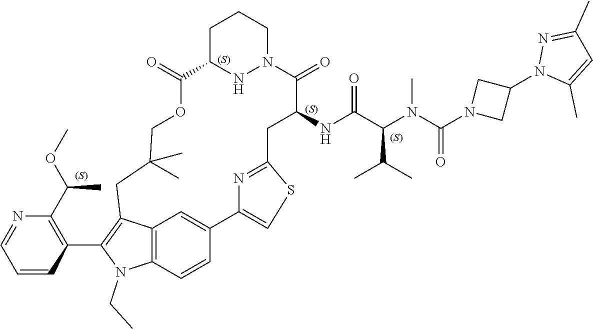 |
| A298 | 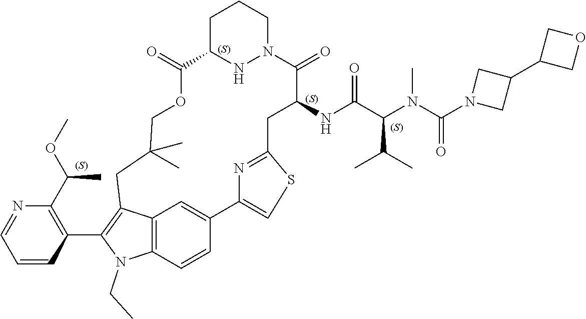 |
| A299 | 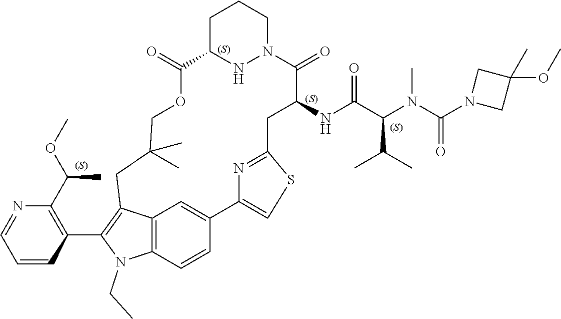 |
| A300 | 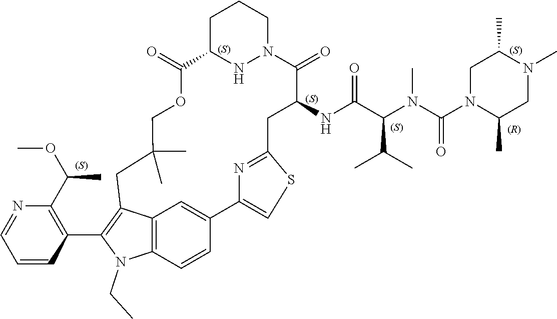 |
| A301 | 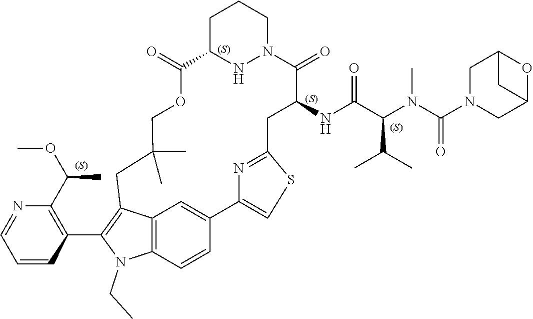 |
| A302 | 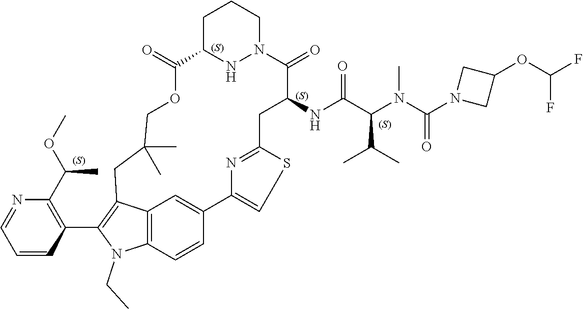 |
| A303 | 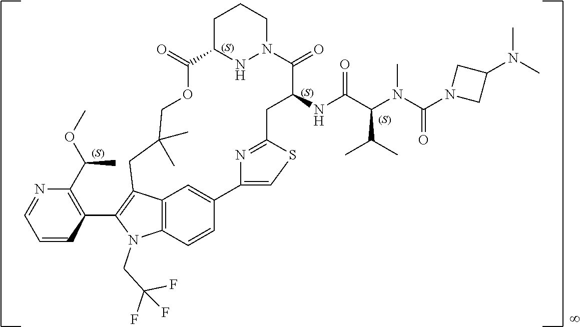 |
| A304 | 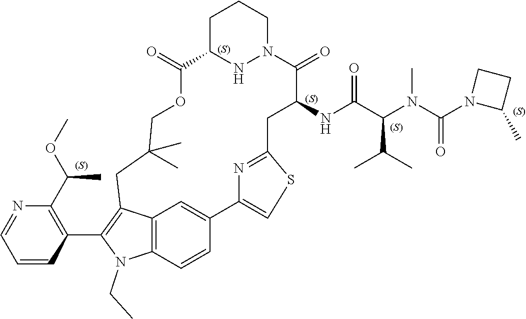 |
| A305 | 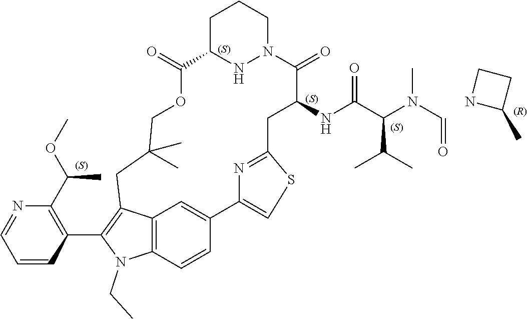 |
| A306 | 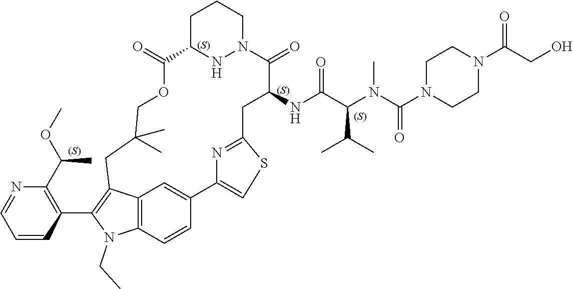 |
| A307 | 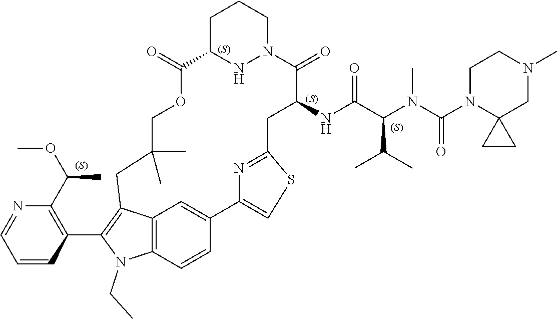 |
| A308 | 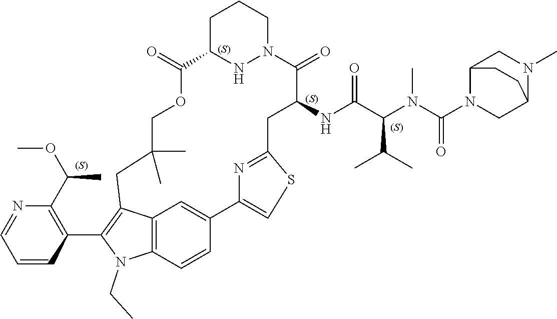 |
| A309 | 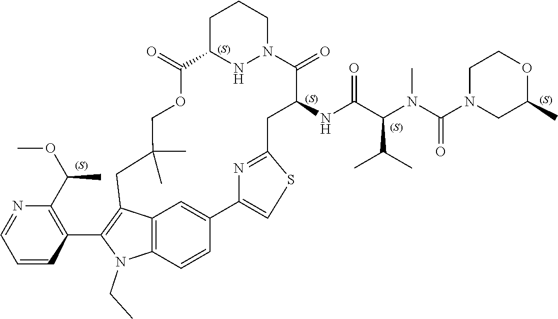 |
| A310 | 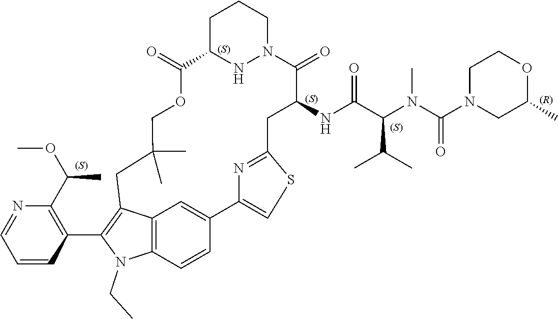 |
| A311 | 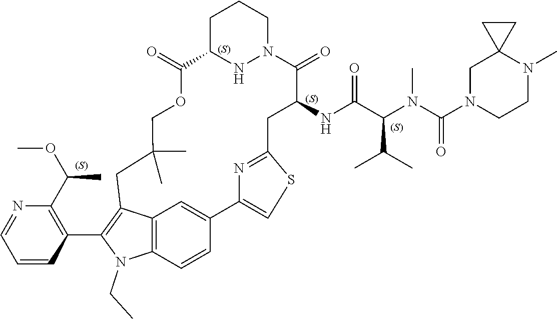 |
| A312 | 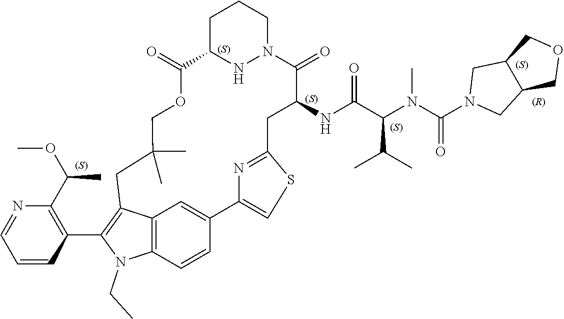 |
| A313 | 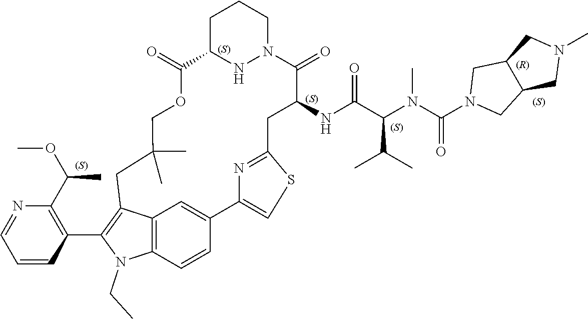 |
| A314 | 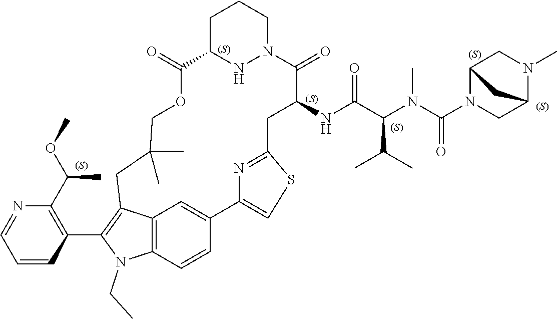 |
| A315 | 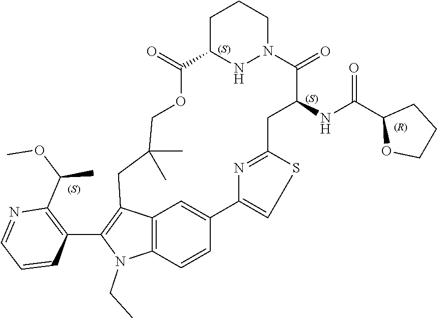 |
| A316 | 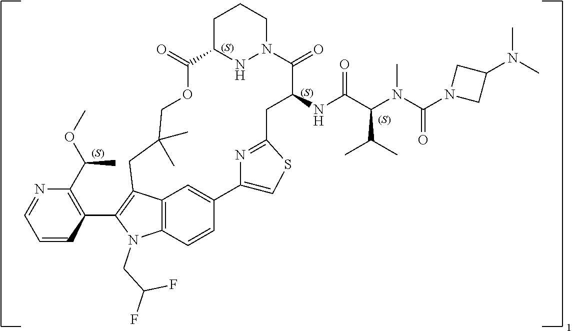 |
| A317 | 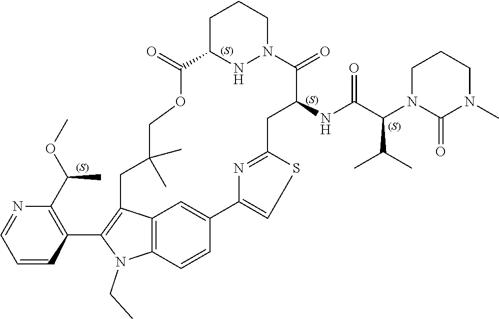 |
| A318 | 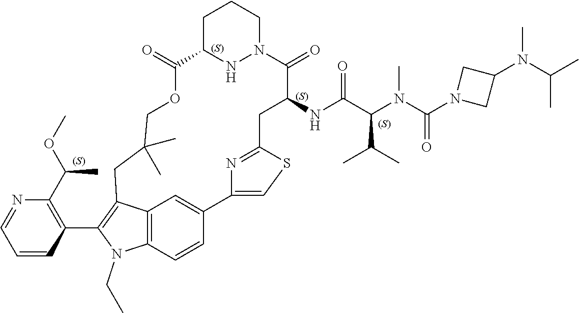 |
| A319 | 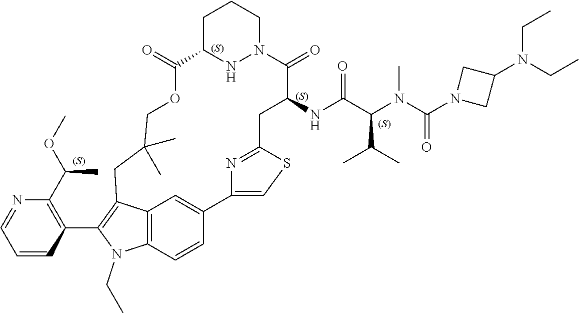 |
| A320 | 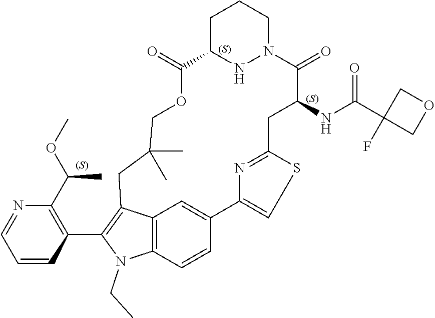 |
| A321 | 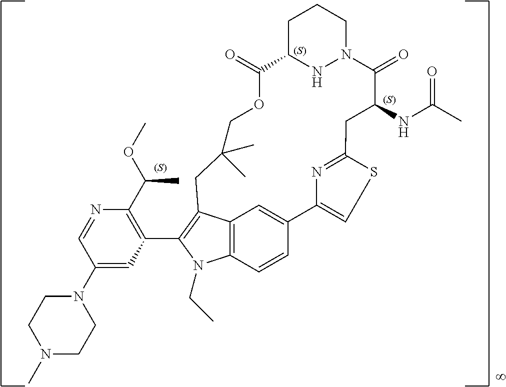 |
| A322 | 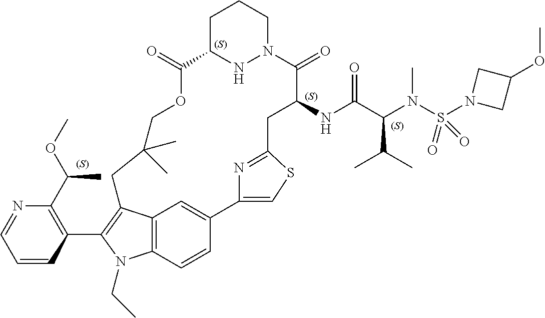 |
| A323 | 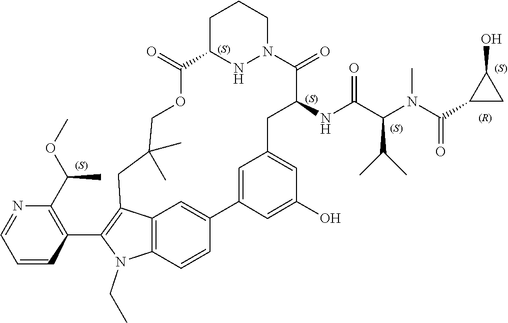 |
| A324 | 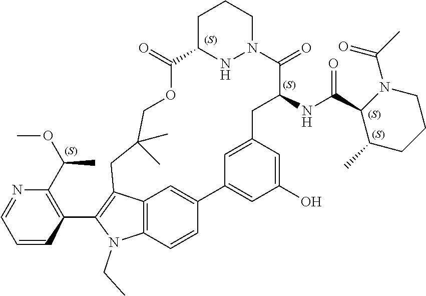 |
| A325 | 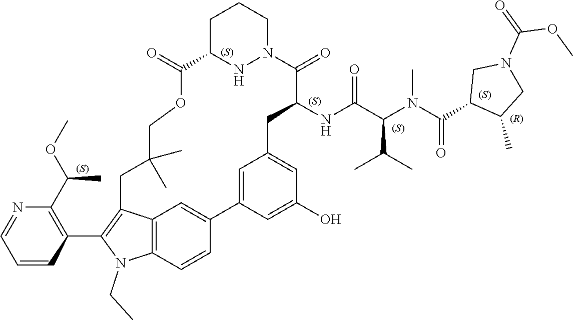 |
| A326 | 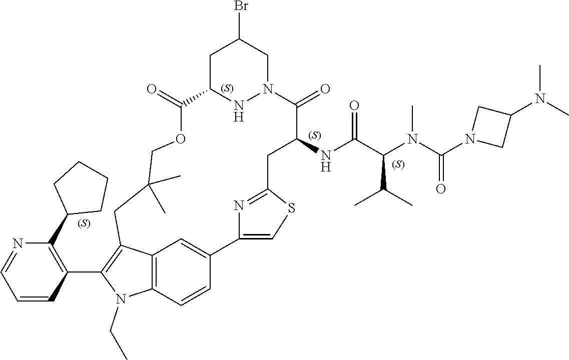 |
| A327 | 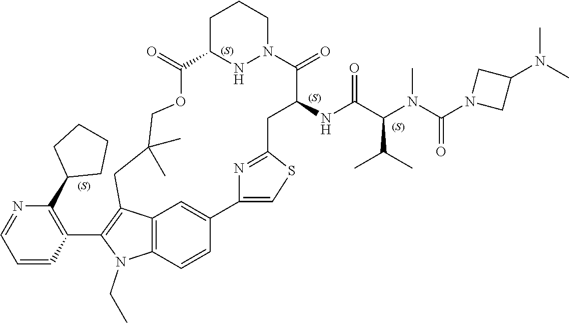 |
| A328 | 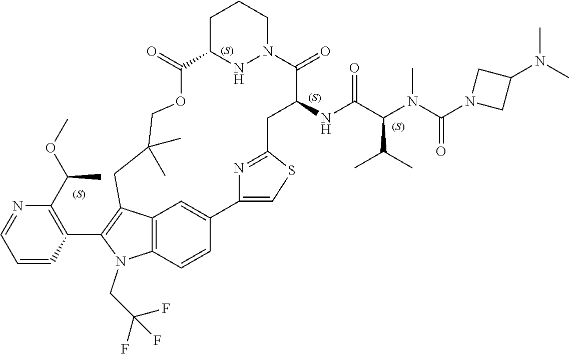 |
| A329 | 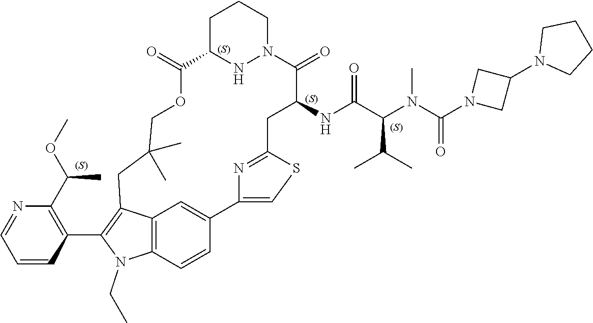 |
| A330 | 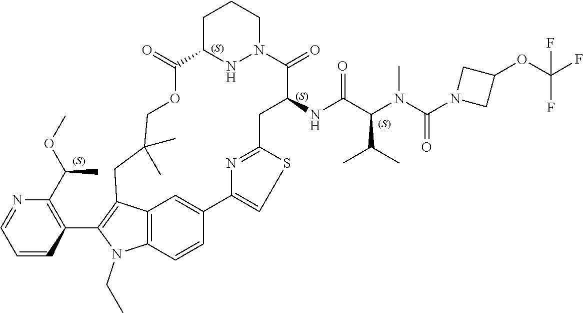 |
| A331 | 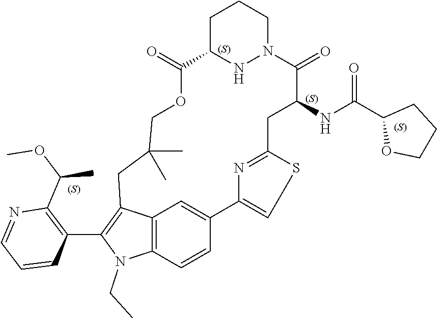 |
| A332 | 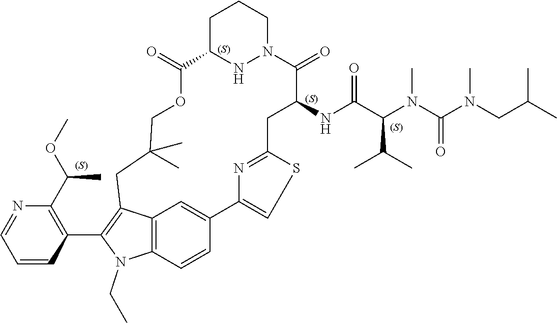 |
| A333 | 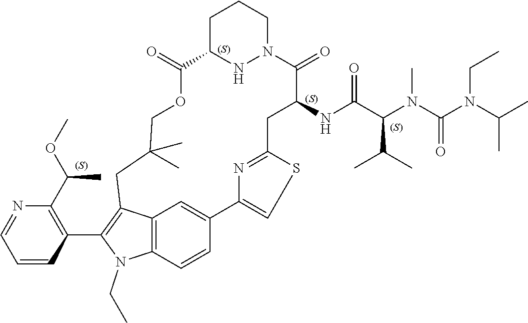 |
| A334 | 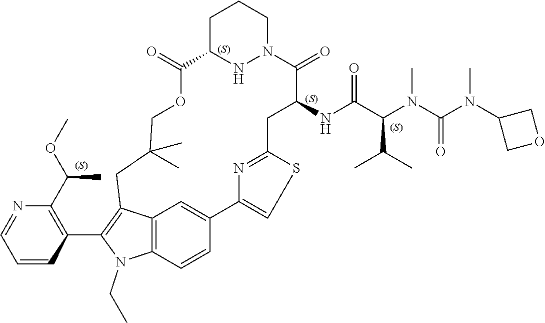 |
| A335 | 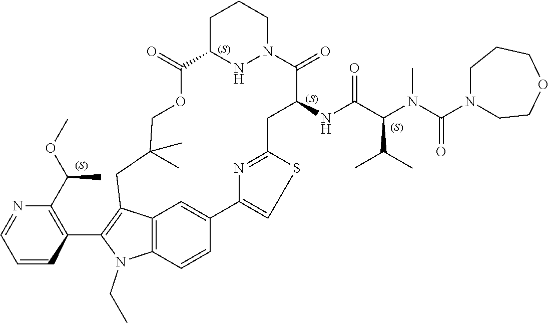 |
| A336 | 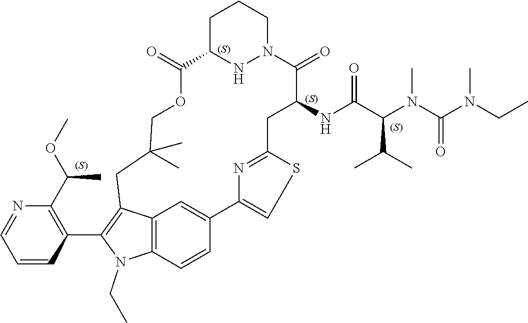 |
| A337 | 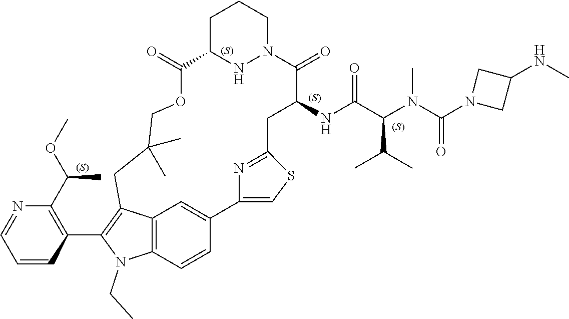 |
| A338 | 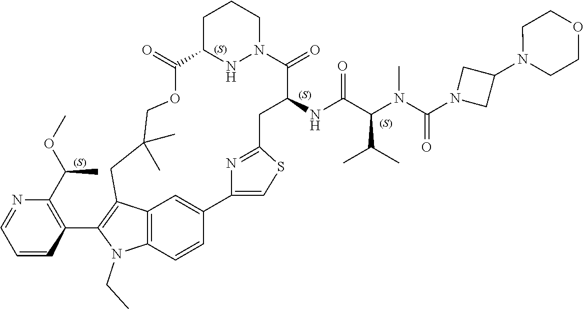 |
| A339 | 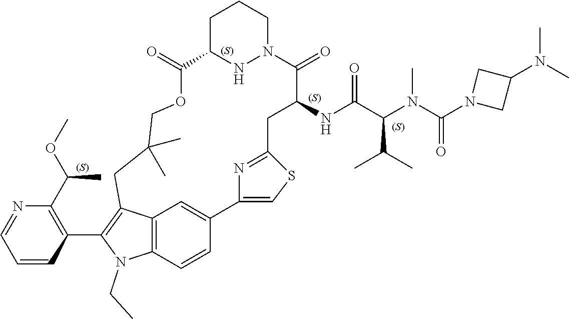 |
| A340 | 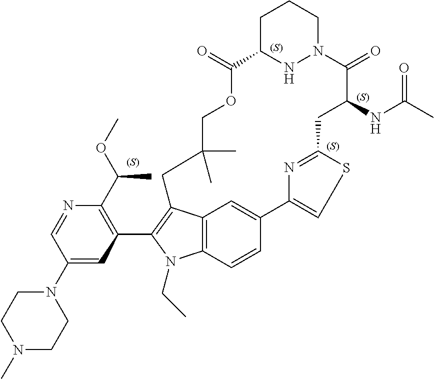 |
| A341 | 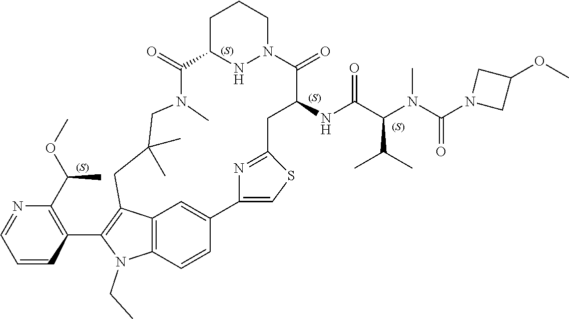 |
| A342 | 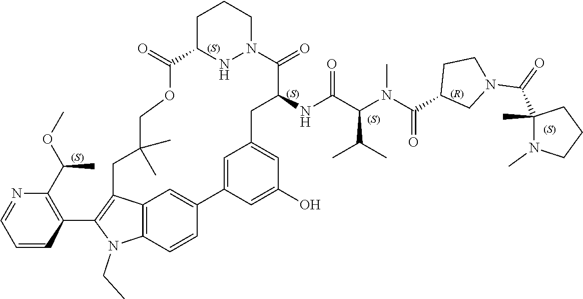 |
| A343 | 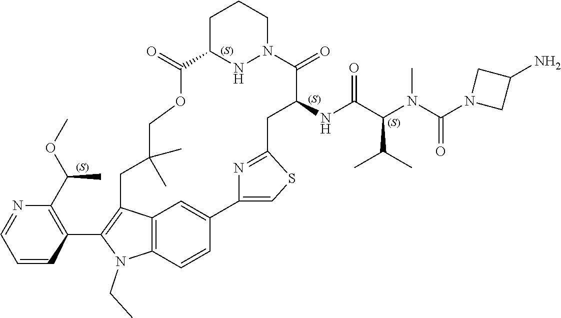 |
| A344 | 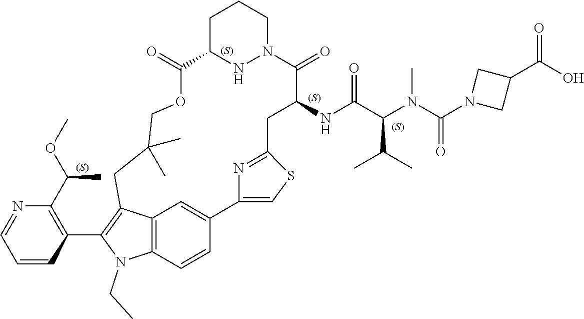 |
| A345 | 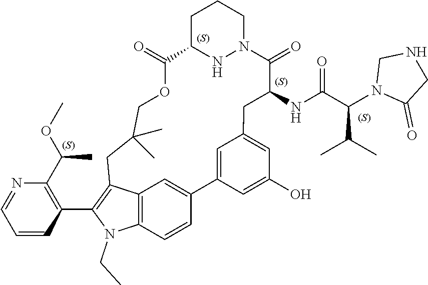 |
| A346 | 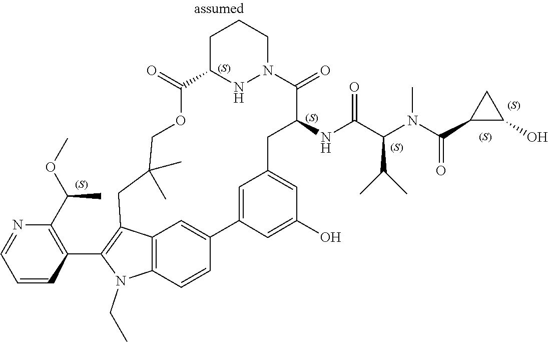 |
| A347 | 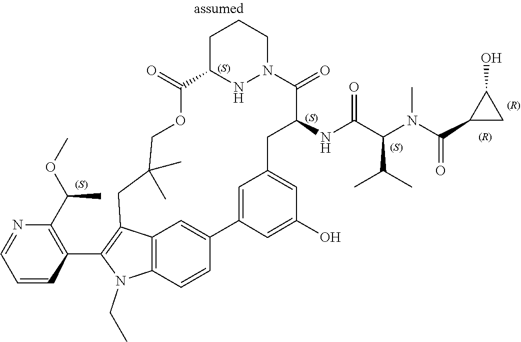 |
| A348 | 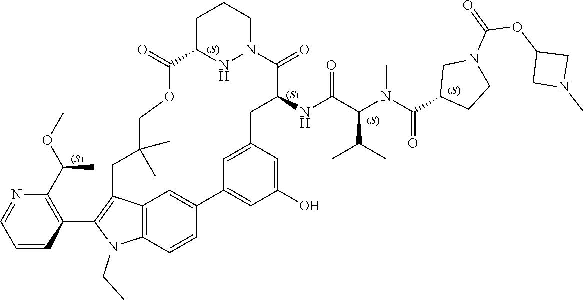 |
| A349 | 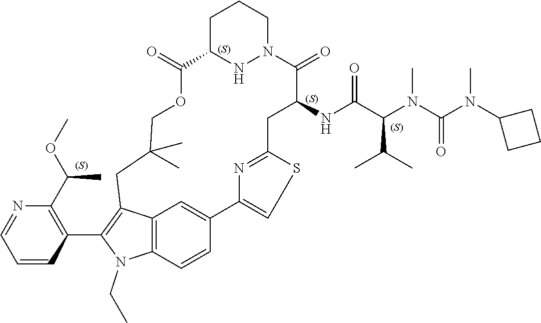 |
| A350 | 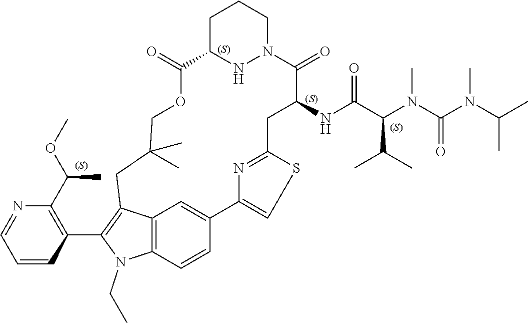 |
| A351 | 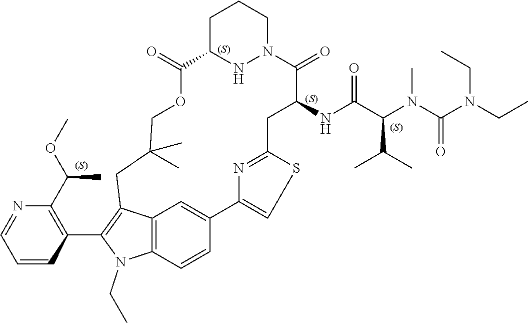 |
| A352 | 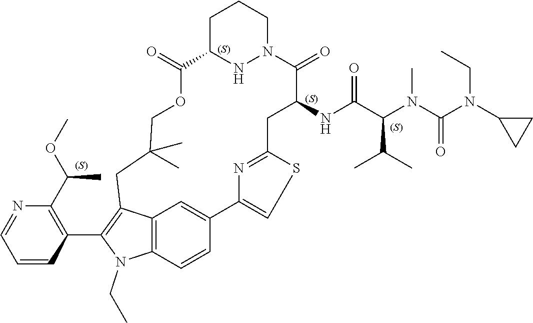 |
| A353 | 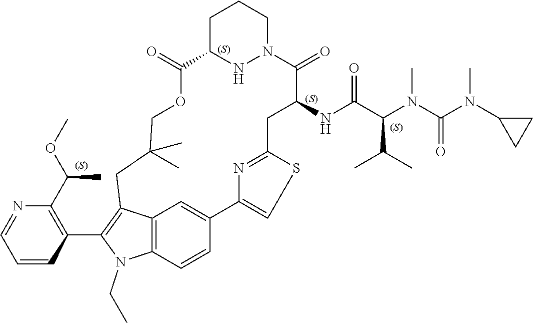 |
| A354 | 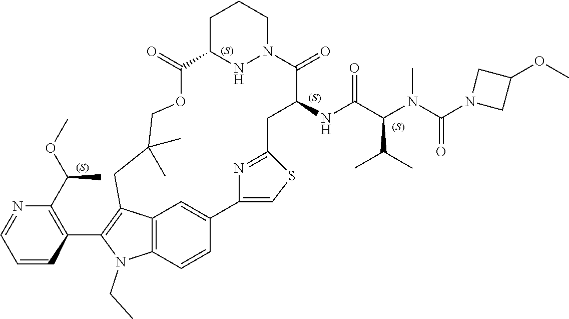 |
| A355 | 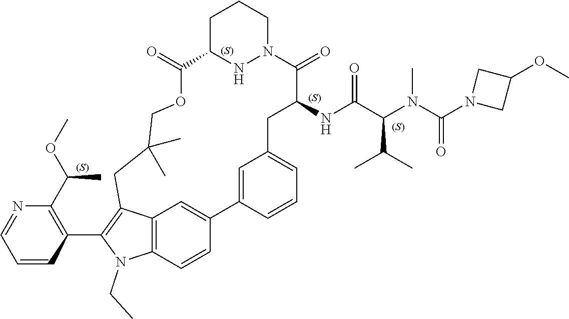 |
| A356 | 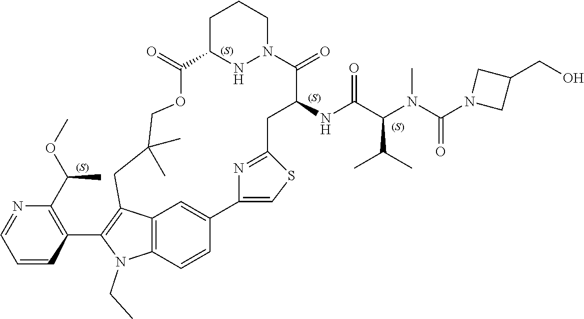 |
| A357 | 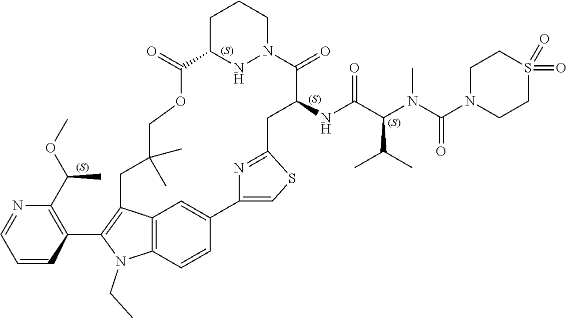 |
| A358 | 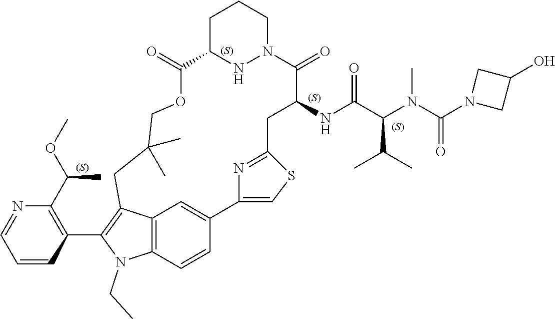 |
| A359 | 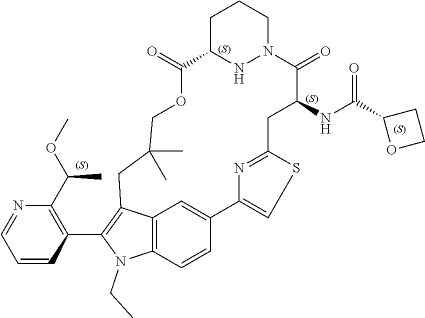 |
| A360 | 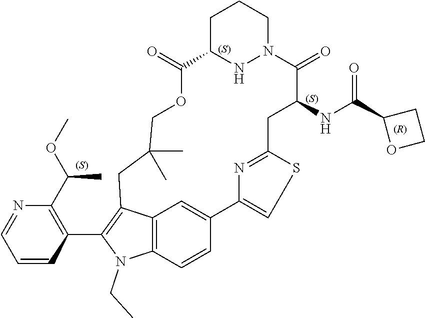 |
| A361 | 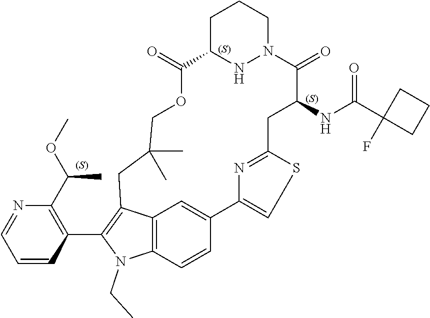 |
| A362 | 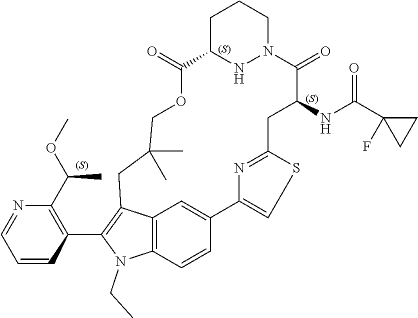 |
| A363 | 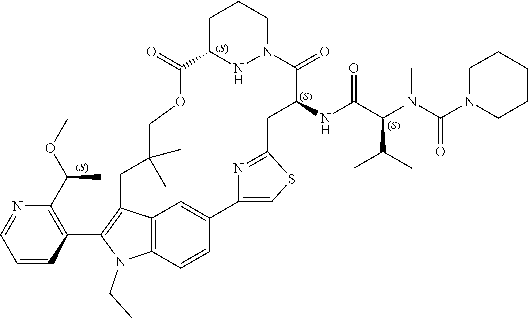 |
| A364 | 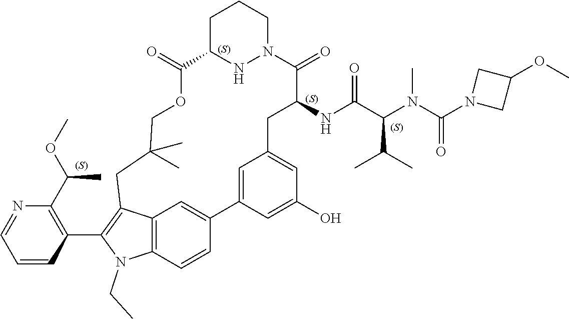 |
| A365 | 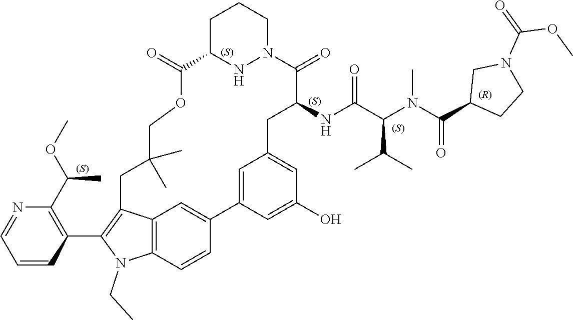 |
| A366 | 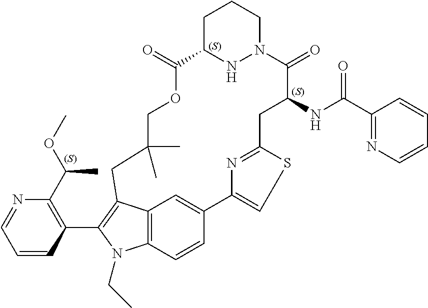 |
| A367 | 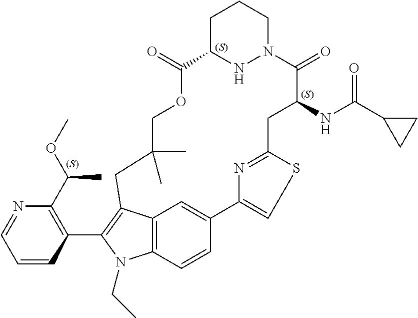 |
| A368 | 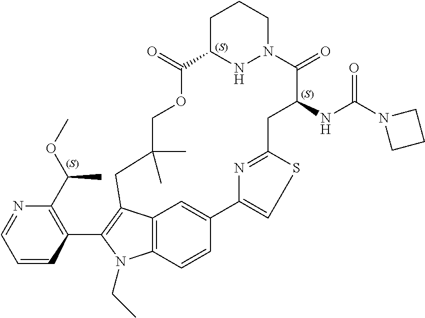 |
| A369 | 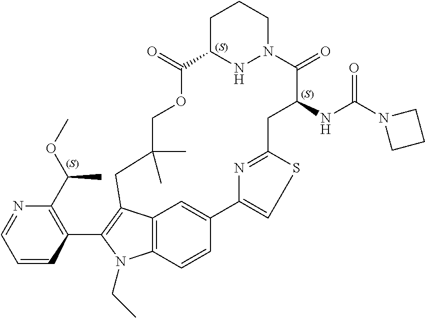 |
| A370 | 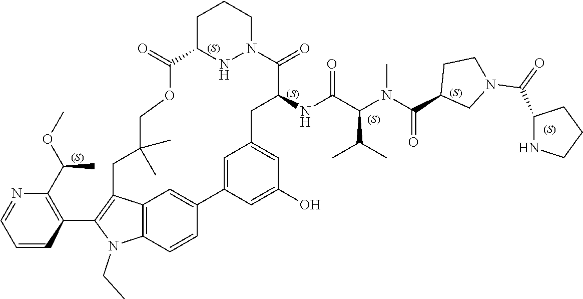 |
| A371 | 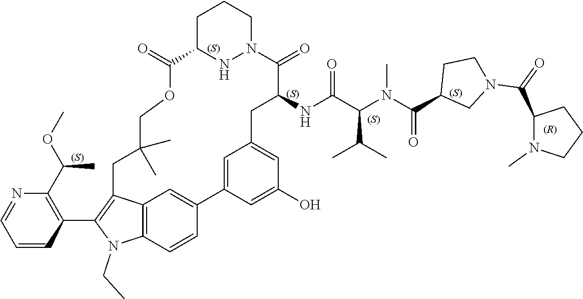 |
| A372 | 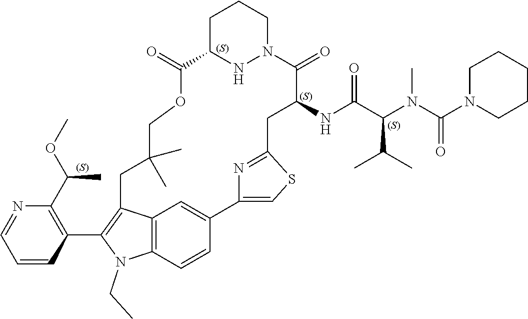 |
| A373 | 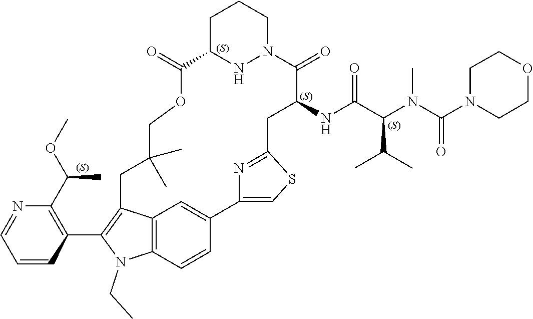 |
| A374 | 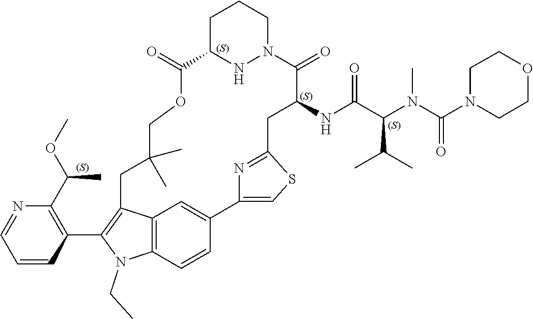 |
| A375 | 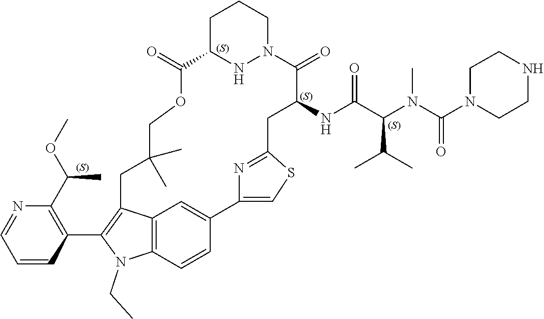 |
| A376 | 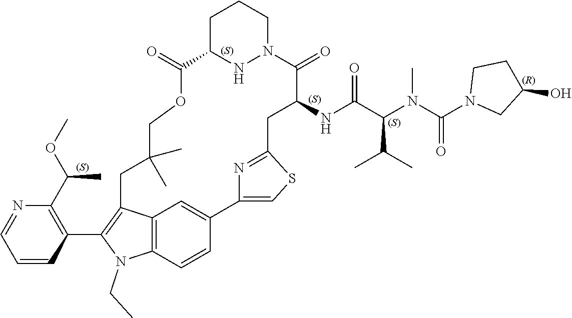 |
| A377 | 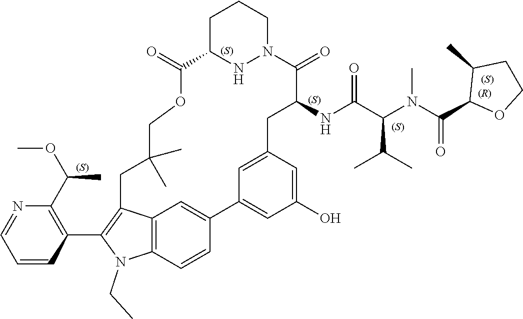 |
| A378 | 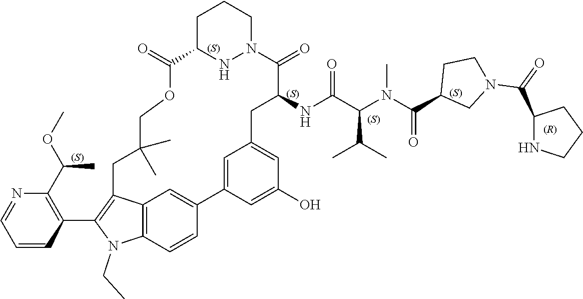 |
| A379 | 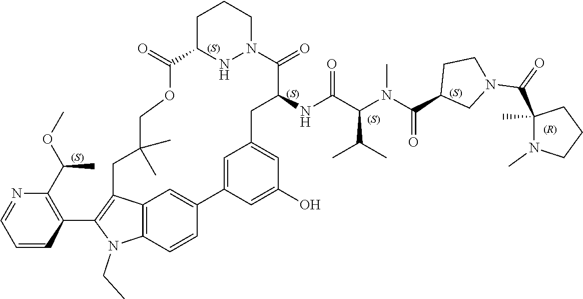 |
| A380 | 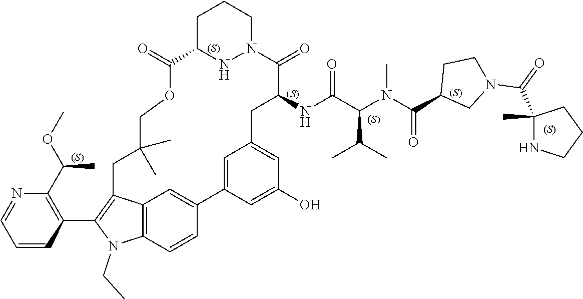 |
| A381 | 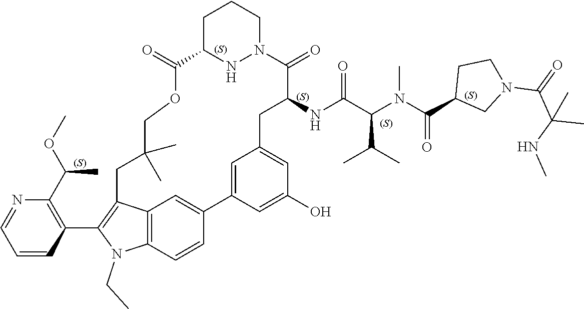 |
| A382 | 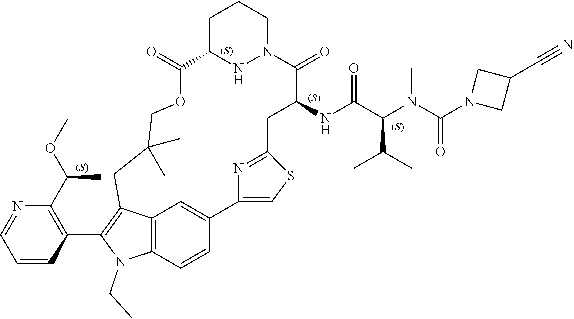 |
| A383 | 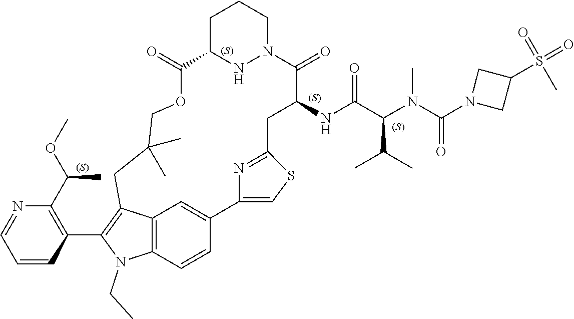 |
| A384 | 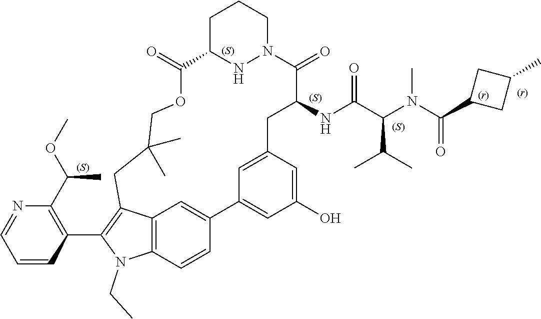 |
| A385 | 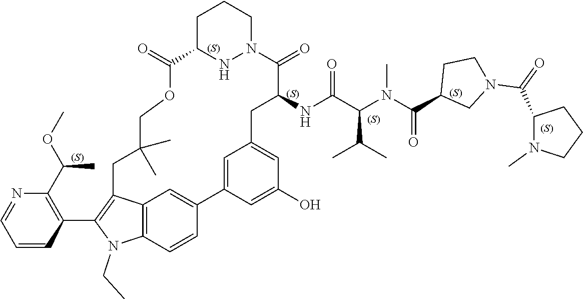 |
| A386 | 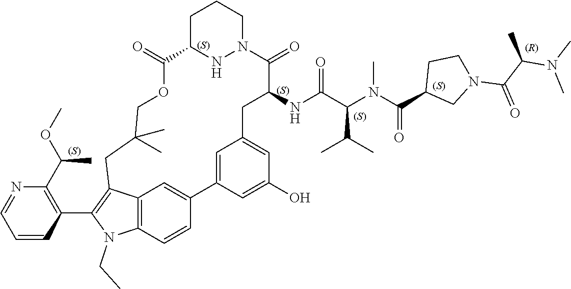 |
| A387 | 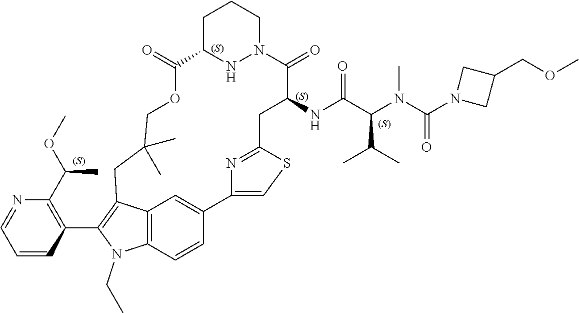 |
| A388 | 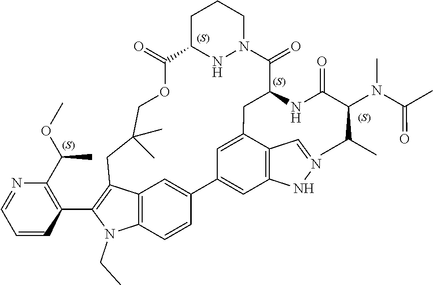 |
| A389 | 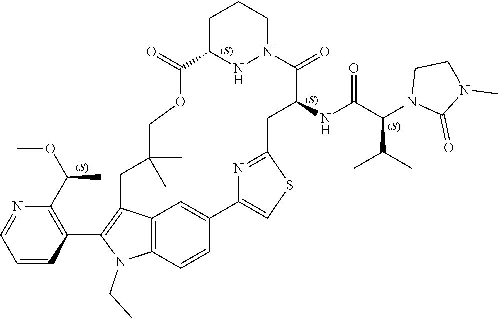 |
| A391 | 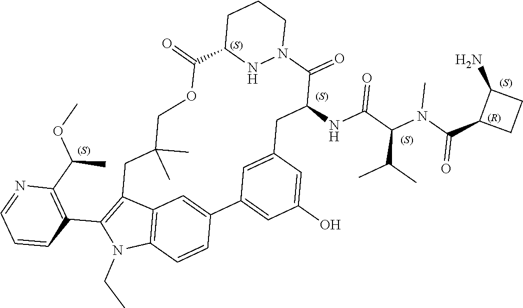 |
| A392 | 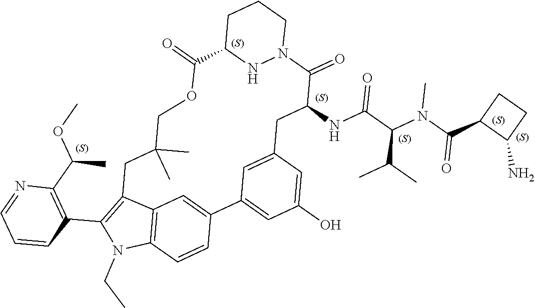 |
| A393 | 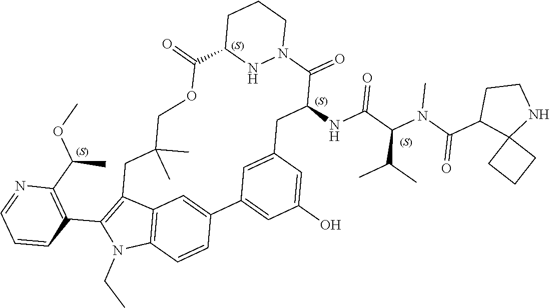 |
| A394 | 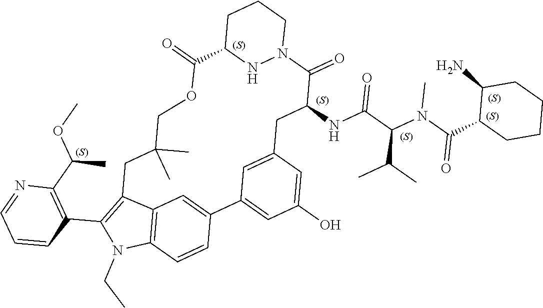 |
| A395 | 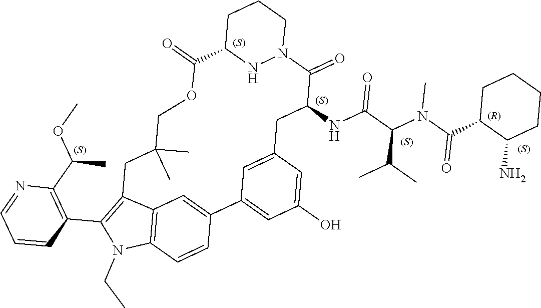 |
| A396 | 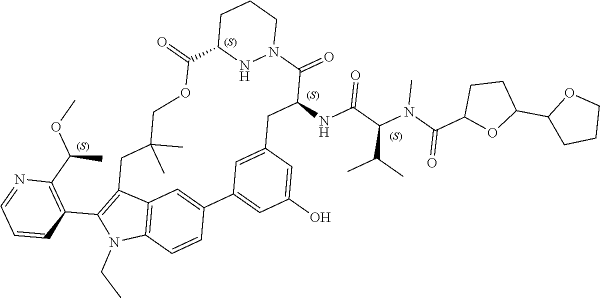 |
| A397 | 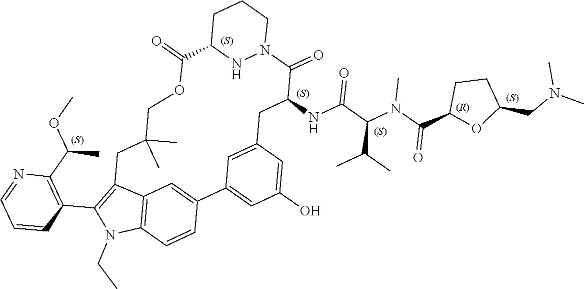 |
| A398 | 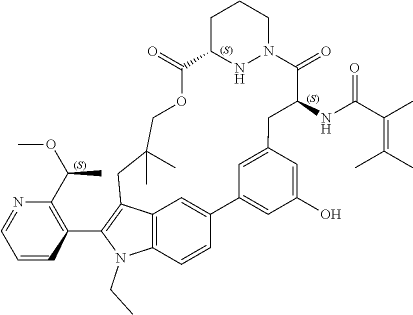 |
| A399 | 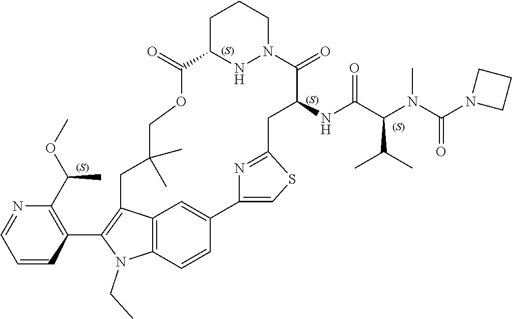 |
| A400 | 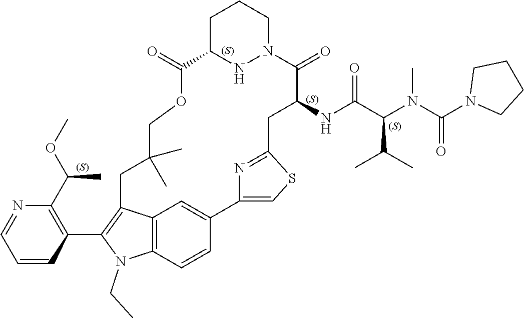 |
| A401 | 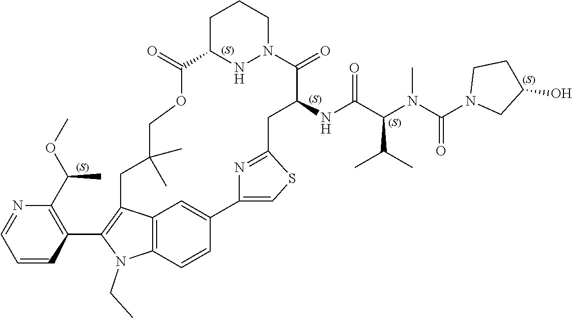 |
| A402 | 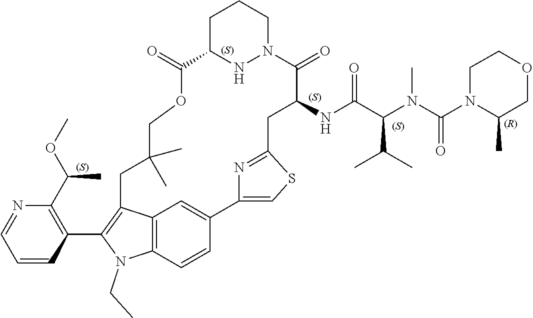 |
| A403 | 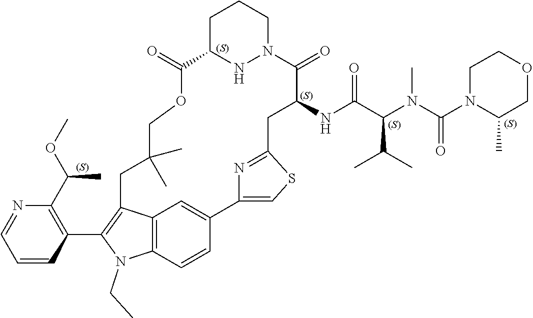 |
| A404 | 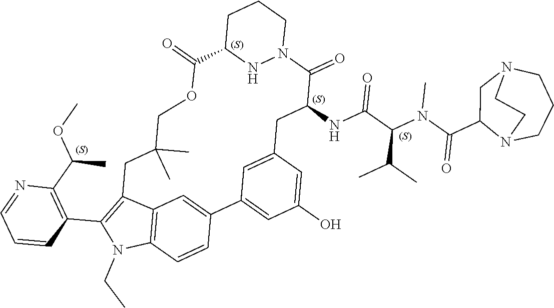 |
| A405 | 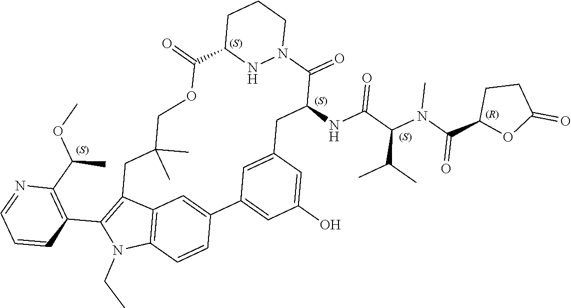 |
| A406 | 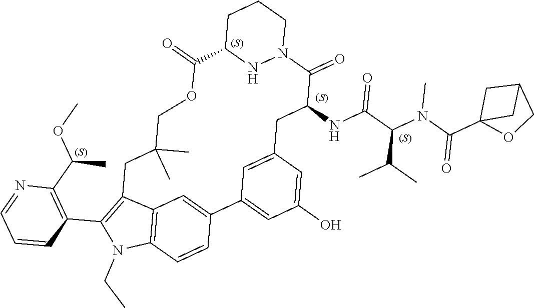 |
| A407 | 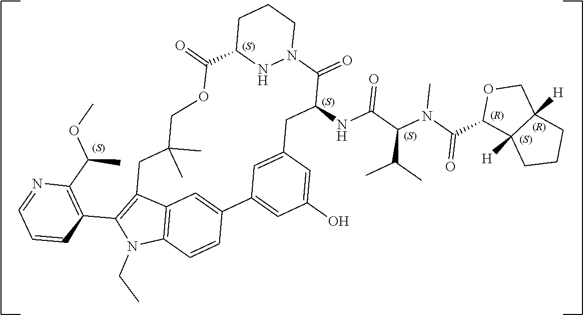 |
| A408 | 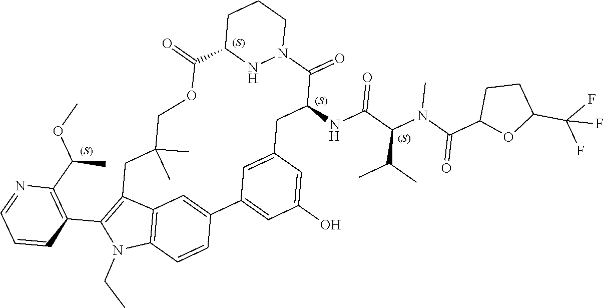 |
| A409 | 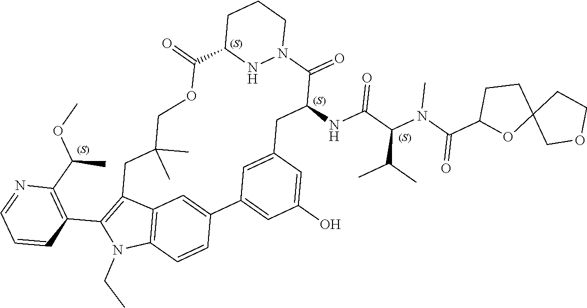 |
| A410 | 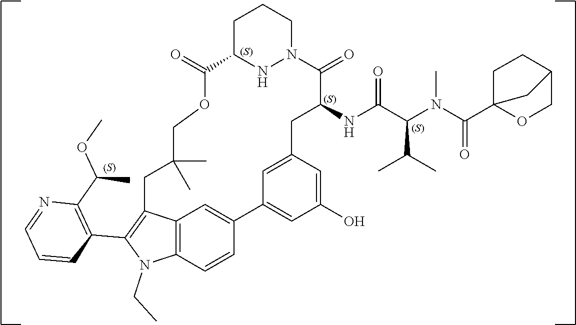 |
| A411 | 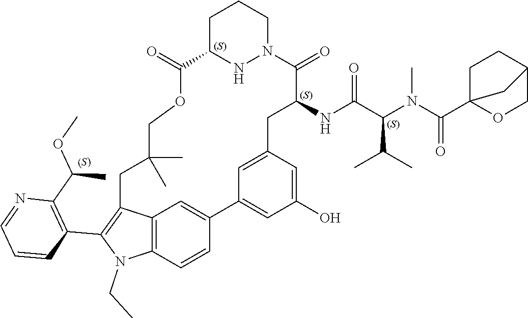 |
| A412 | 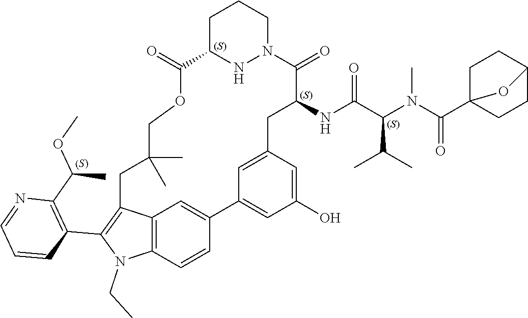 |
| A413 | 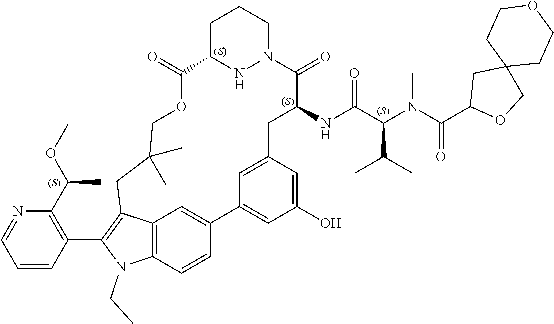 |
| A414 | 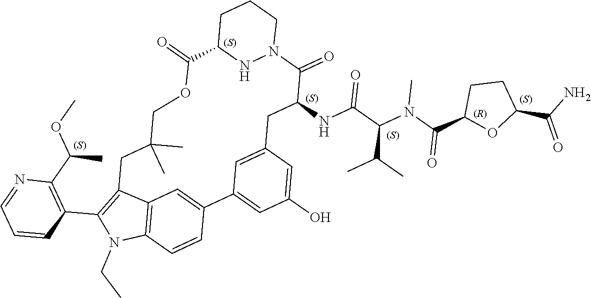 |
| A415 | 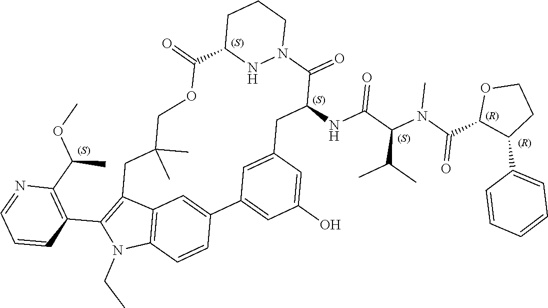 |
| A416 | 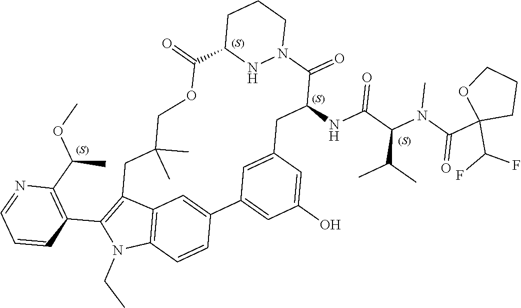 |
| A417 | 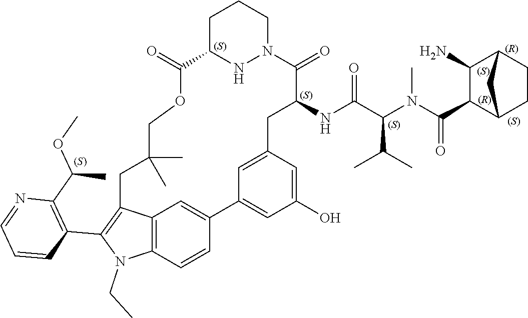 |
| A418 | 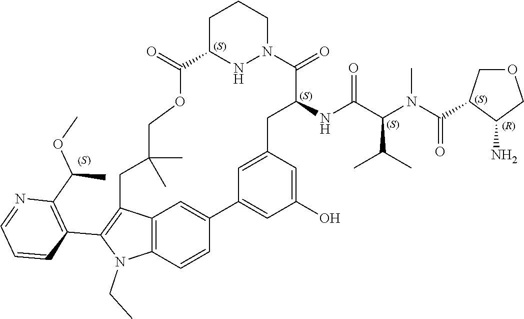 |
| A419 | 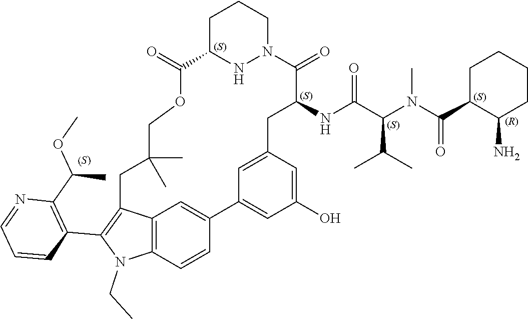 |
| A420 | 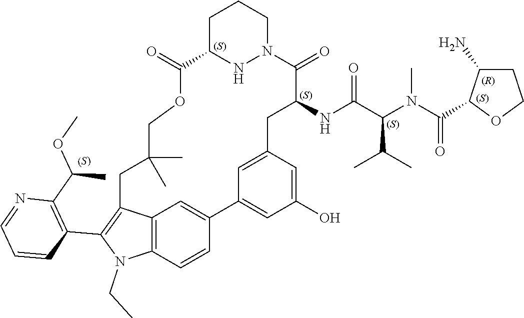 |
| A421 | 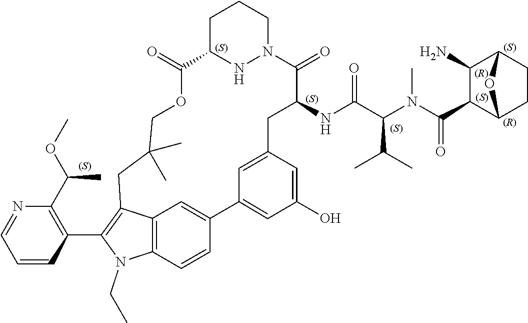 |
| A422 | 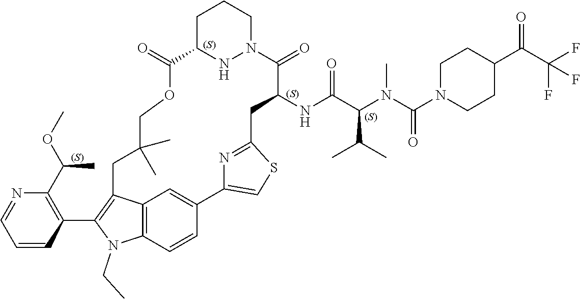 |
| A423 | 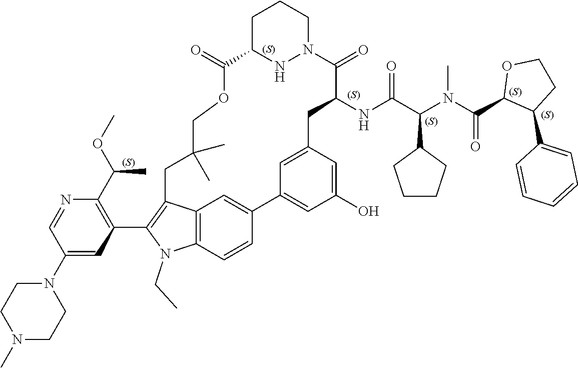 |
| A424 | 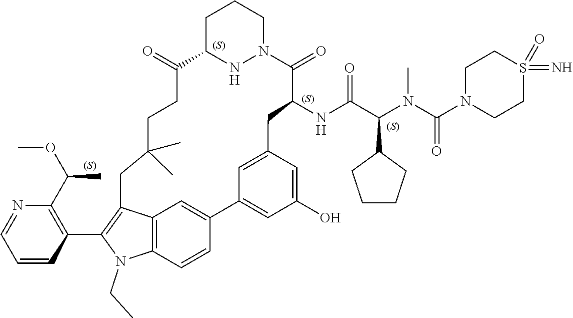 |
| A425 | 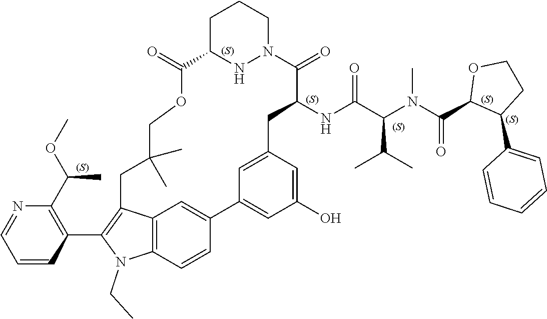 |
| A426 | 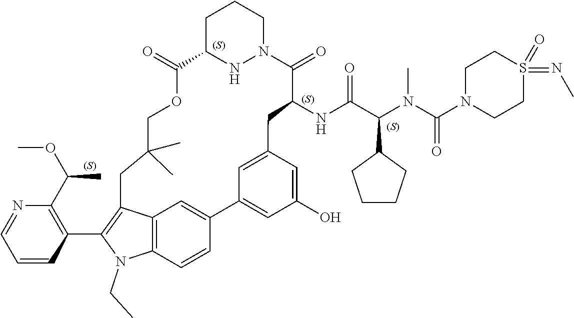 |
| A427 | 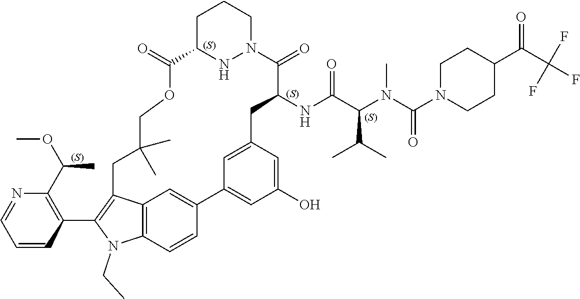 |
| A428 | 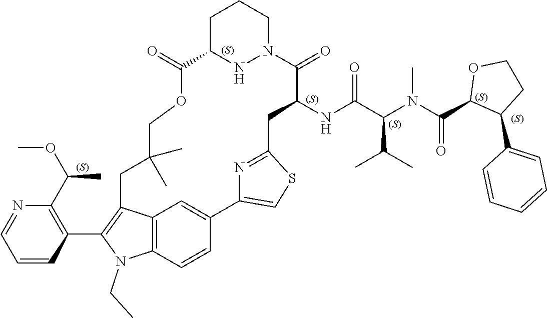 |
| A429 | 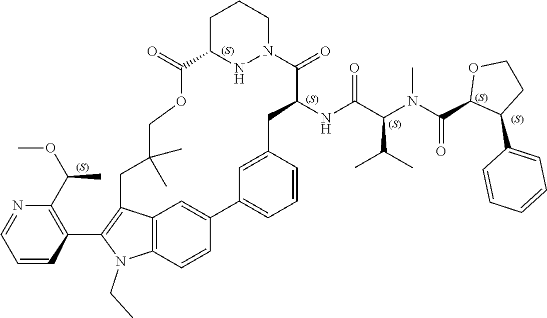 |
| A430 | 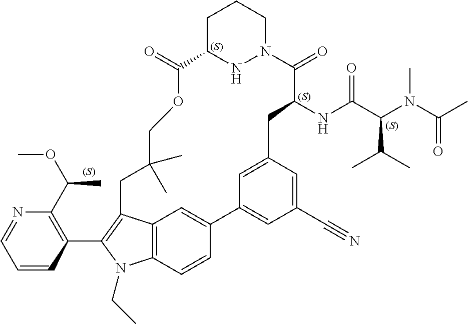 |
| A431 | 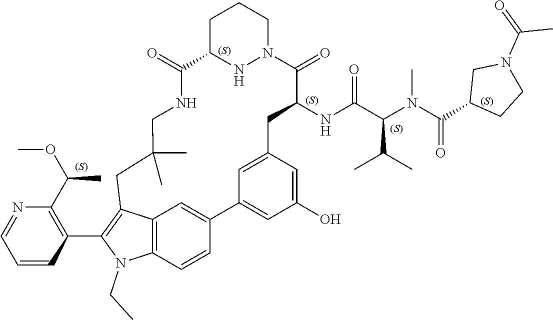 |
| A432 | 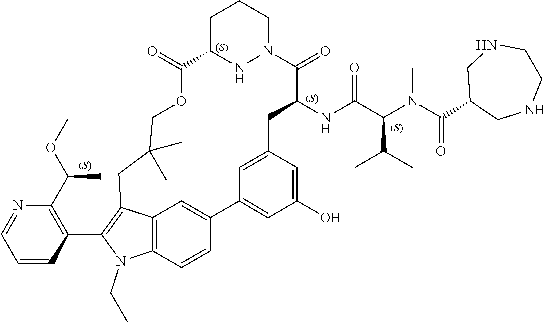 |
| A433 | 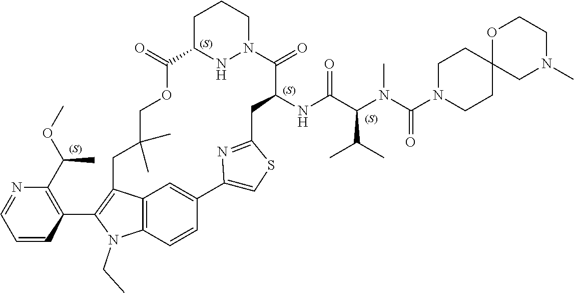 |
| A434 | 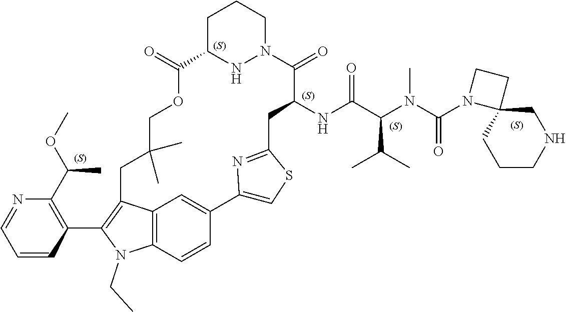 |
| A435 | 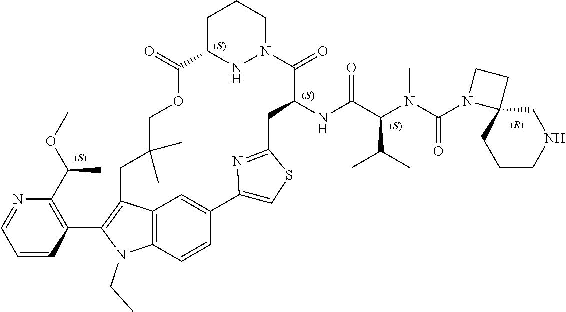 |
| A436 | 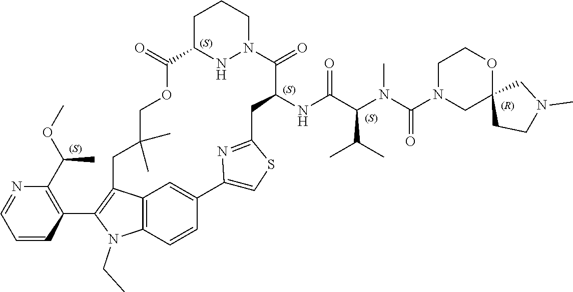 |
| A437 | 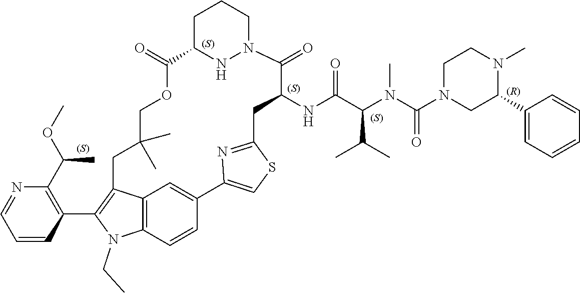 |
| A438 | 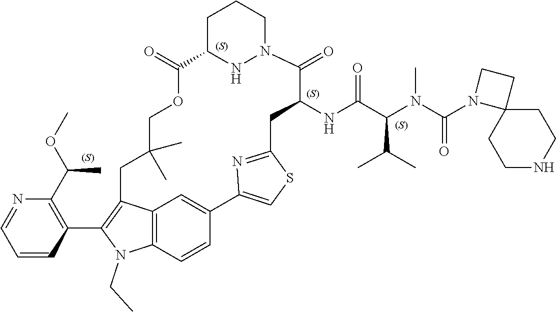 |
| A439 | 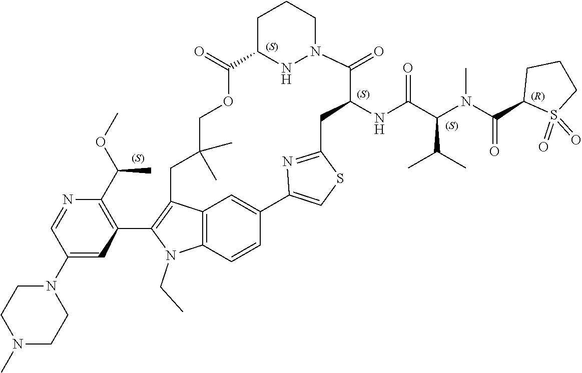 |
| A440 | 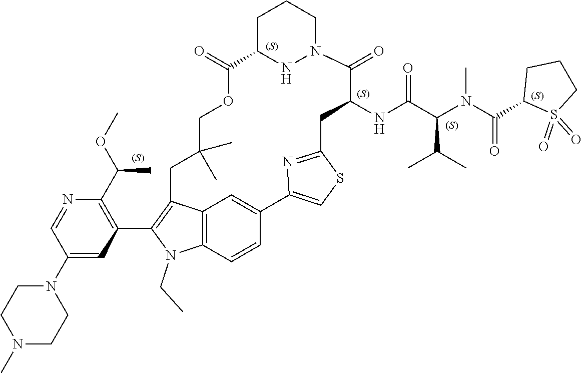 |
| A441 | 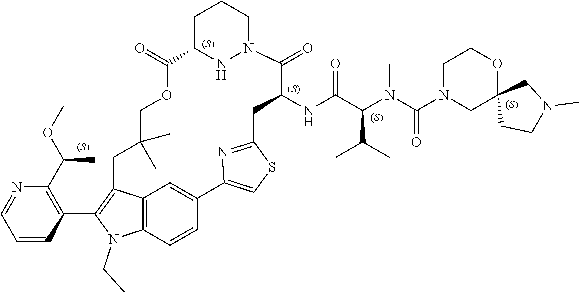 |
| A442 | 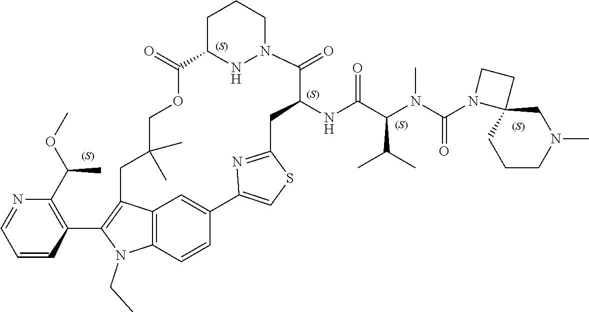 |
| A443 | 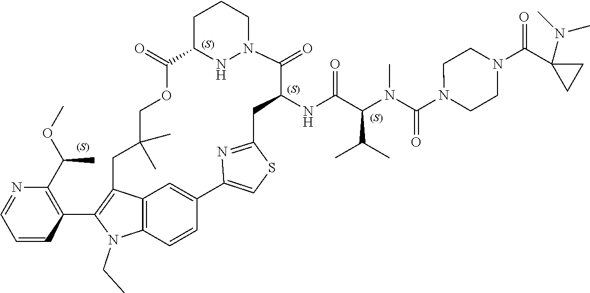 |
| A444 | 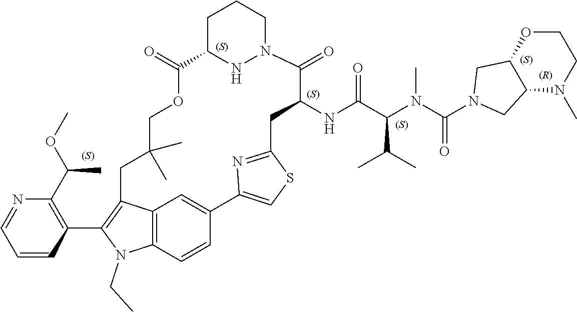 |
| A445 | 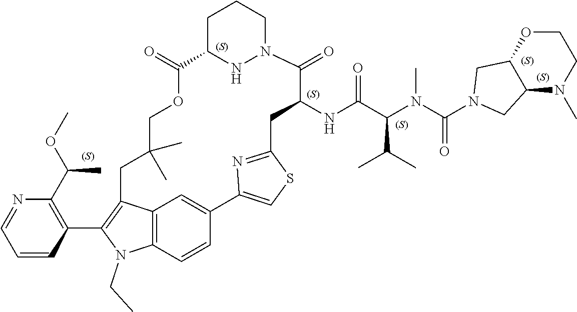 |
| A446 | 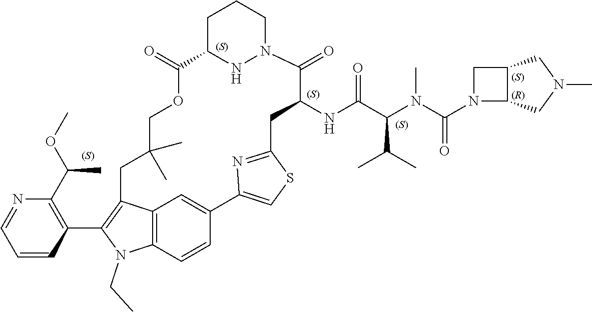 |
| A447 | 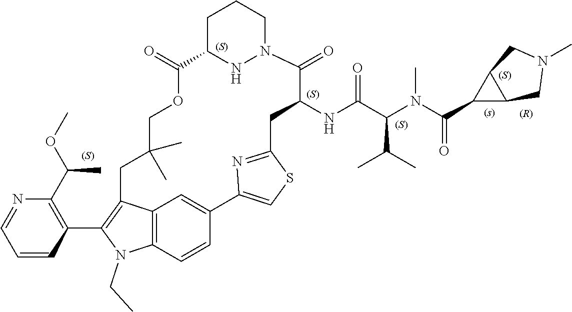 |
| A448 | 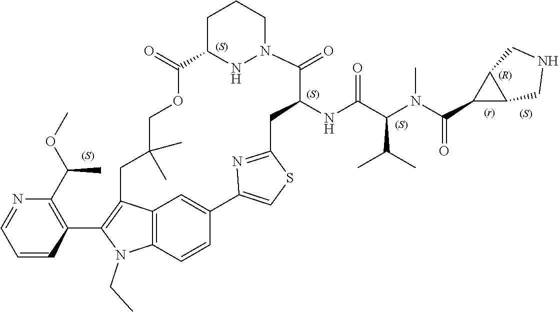 |
| A449 | 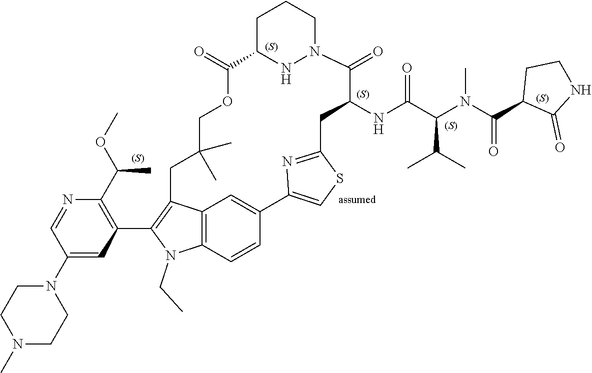 |
| A450 | 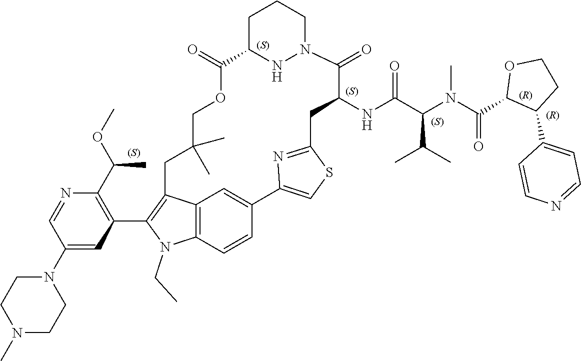 |
| A451 | 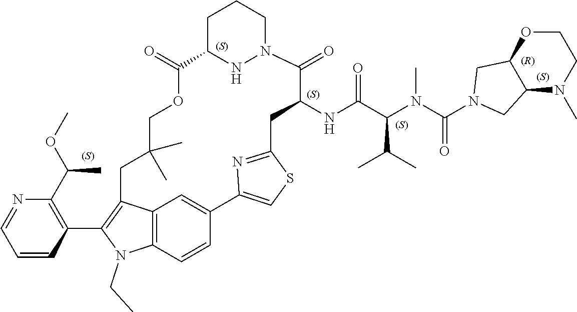 |
| A452 | 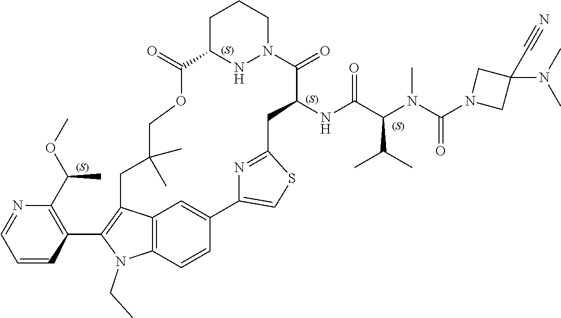 |
| A453 | 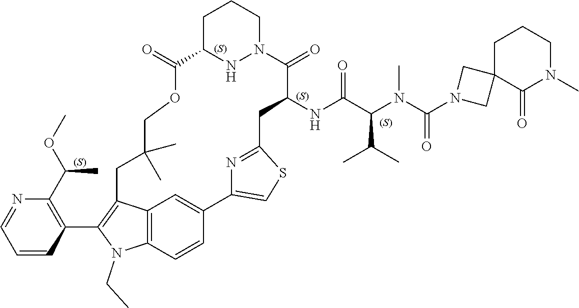 |
| A454 | 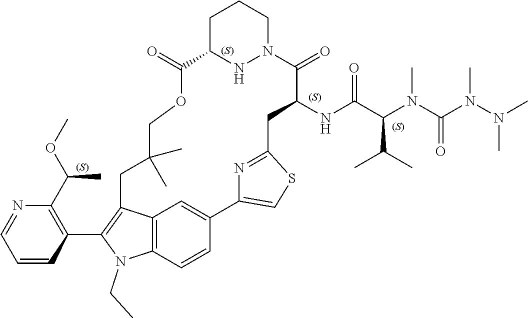 |
| A455 | 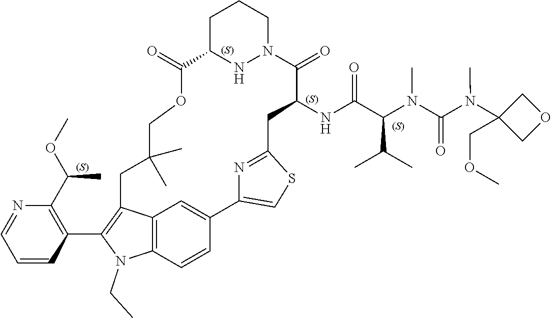 |
| A456 | 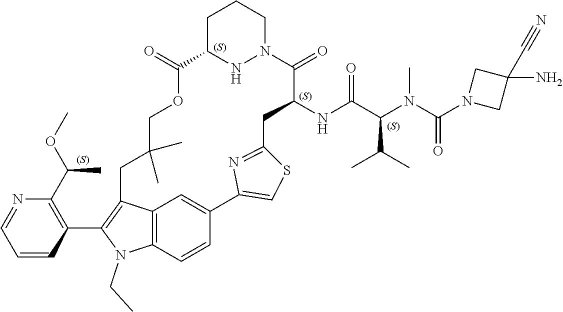 |
| A457 | 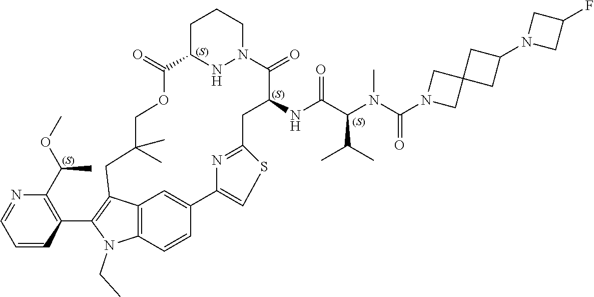 |
| A458 | 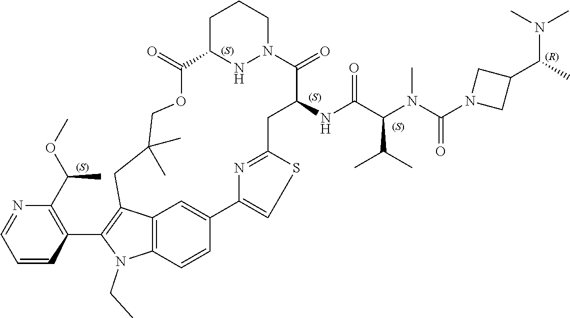 |
| A459 | 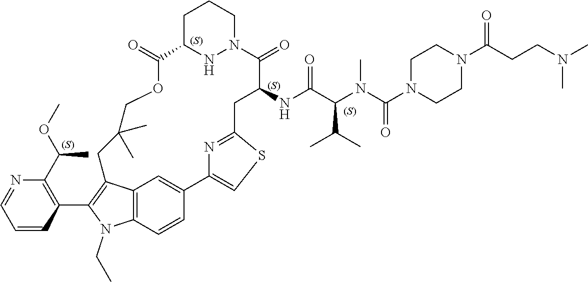 |
| A460 | 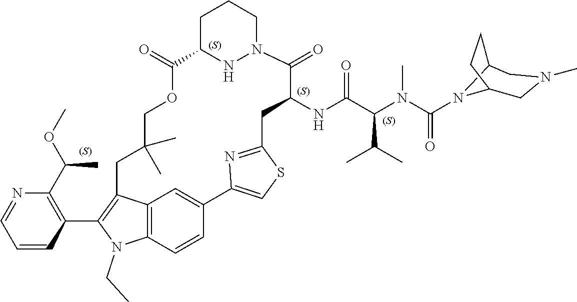 |
| A461 | 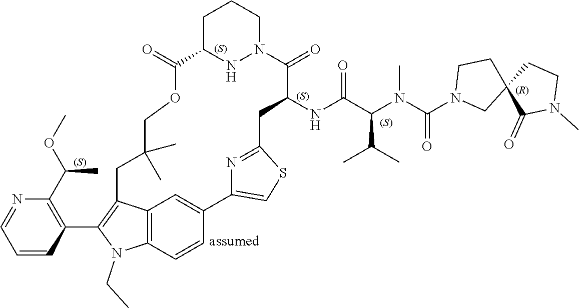 |
| A462 | 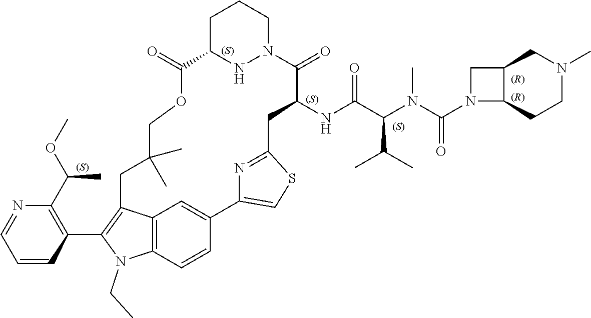 |
| A463 | 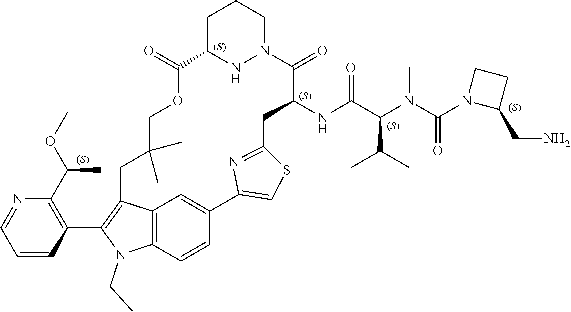 |
| A464 | 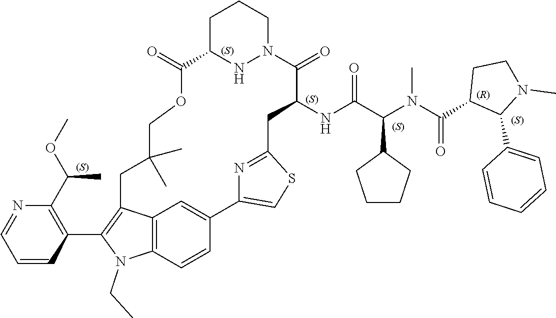 |
| A465 | 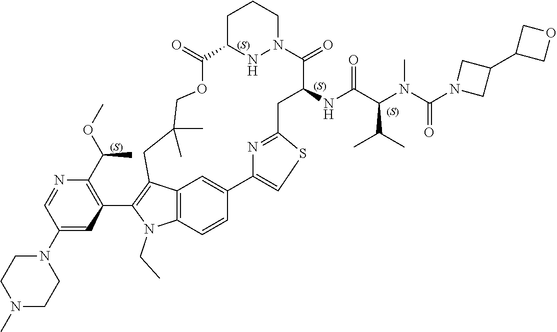 |
| A466 | 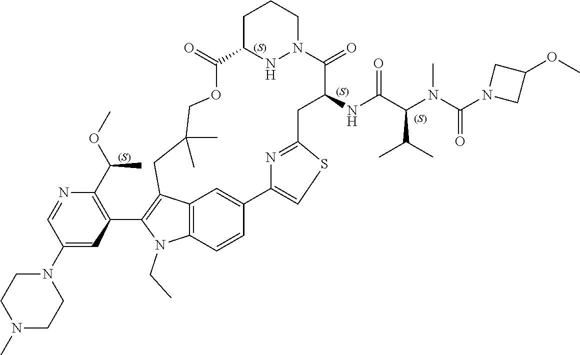 |
| A467 | 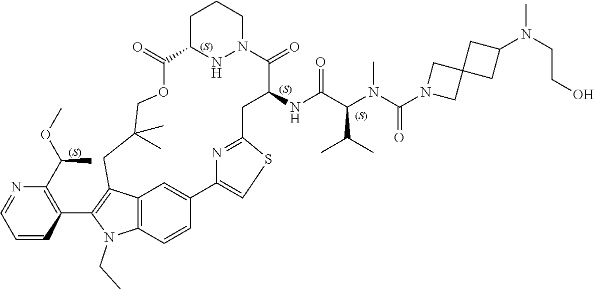 |
| A468 | 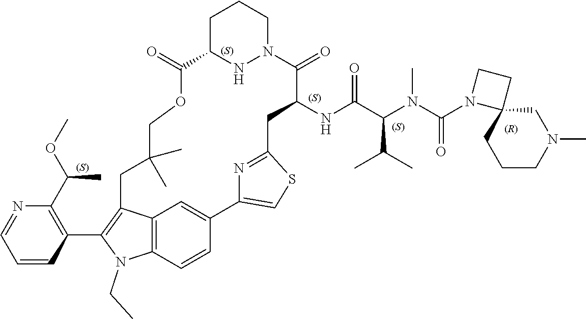 |
| A469 | 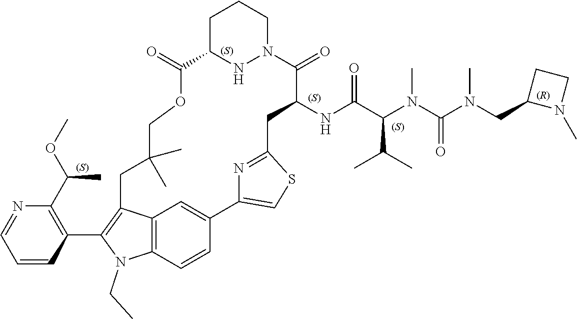 |
| A470 | 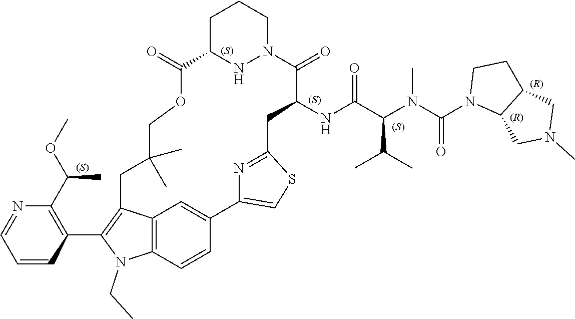 |
| A471 | 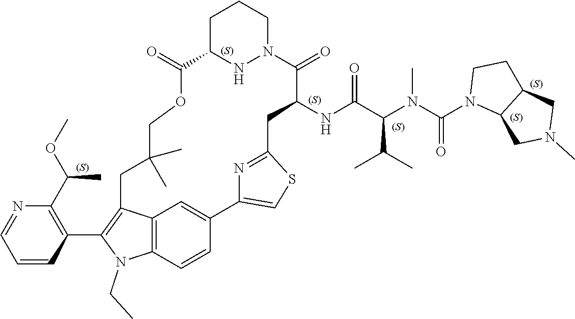 |
| A472 | 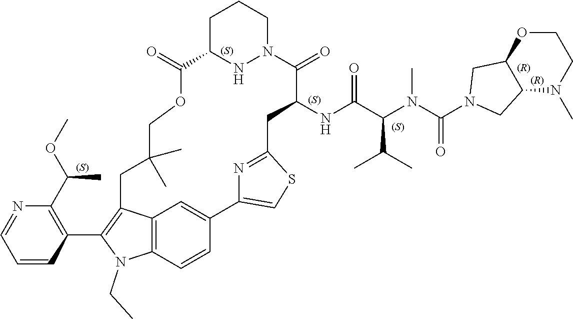 |
| A473 | 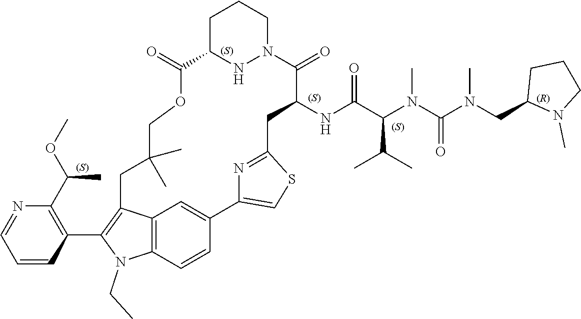 |
| A474 | 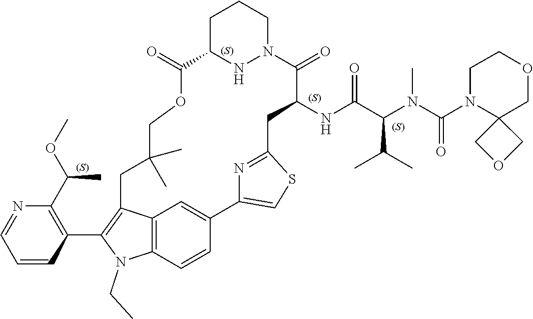 |
| A475 | 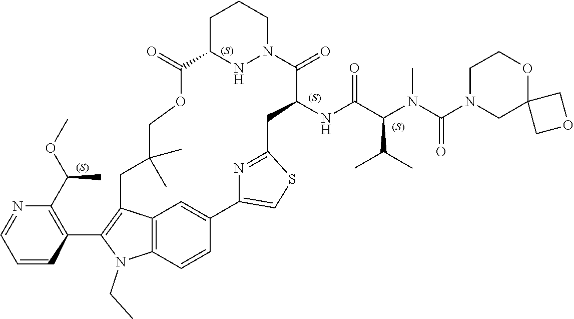 |
| A476 | 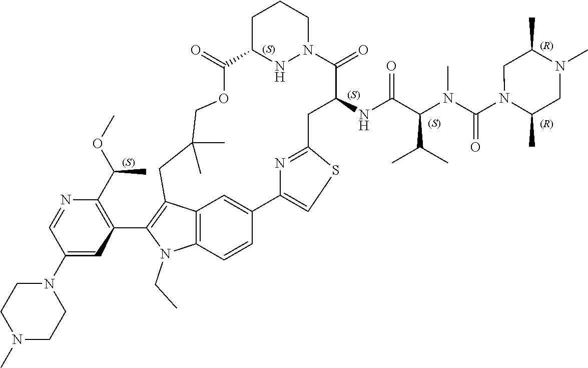 |
| A477 | 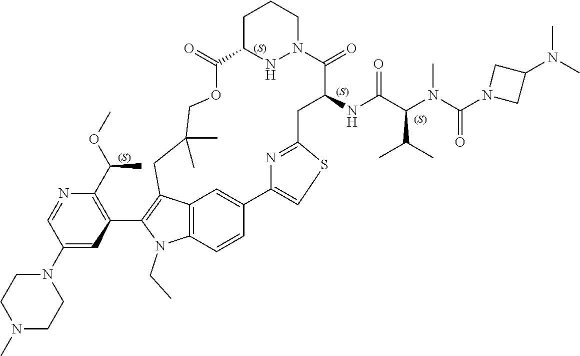 |
| A478 | 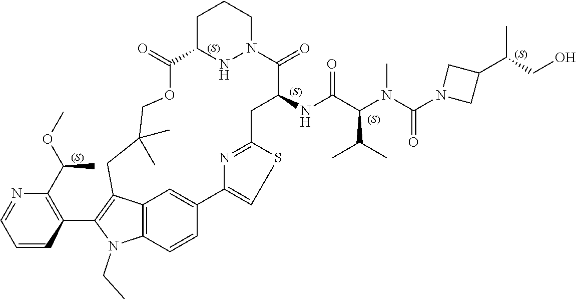 |
| A479 | 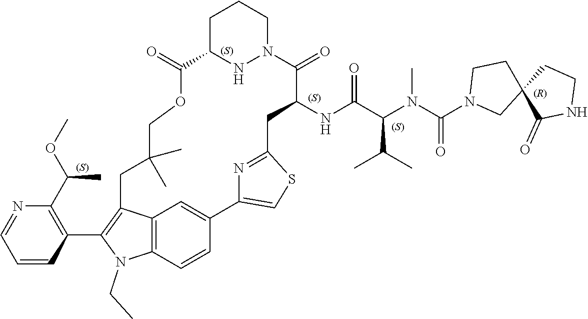 |
| A480 | 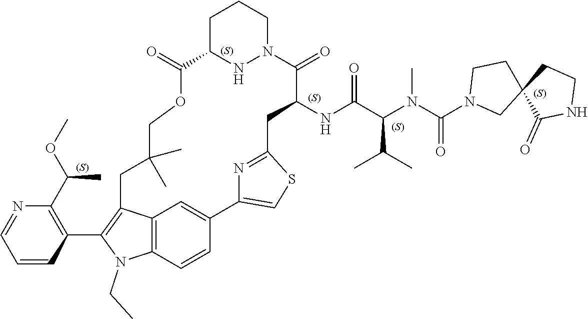 |
| A481 | 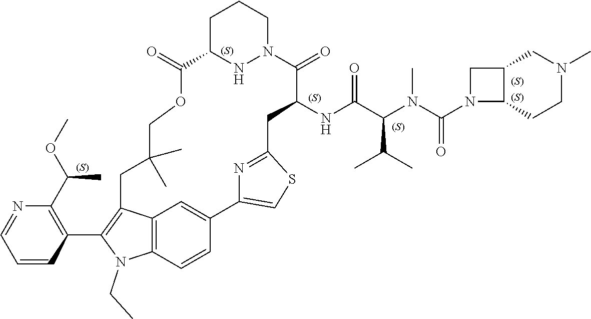 |
| A482 | 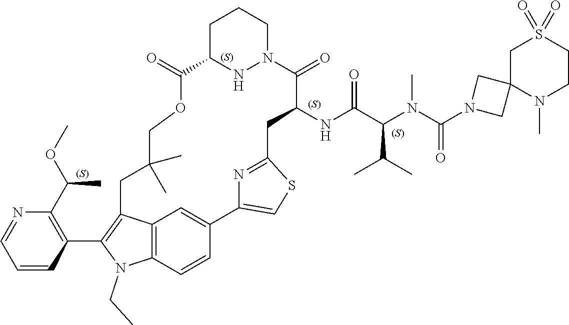 |
| A483 | 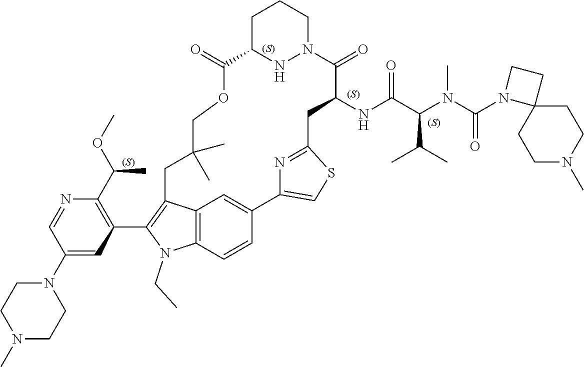 |
| A484 | 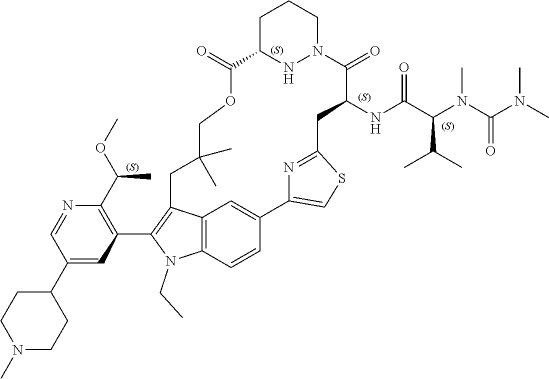 |
| A485 | 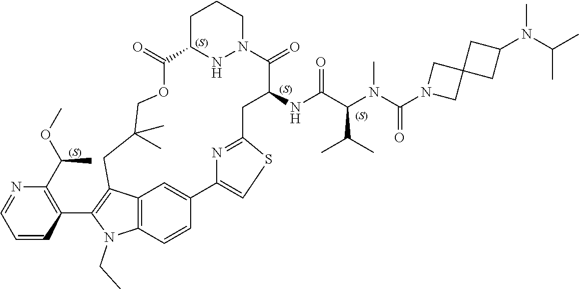 |
| A486 | 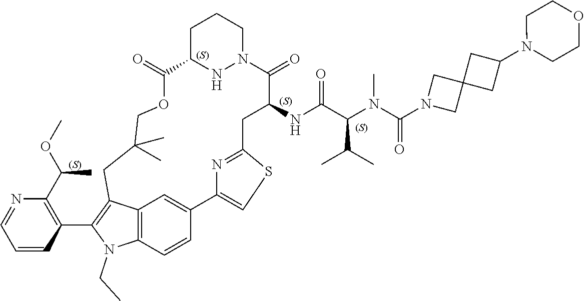 |
| A487 | 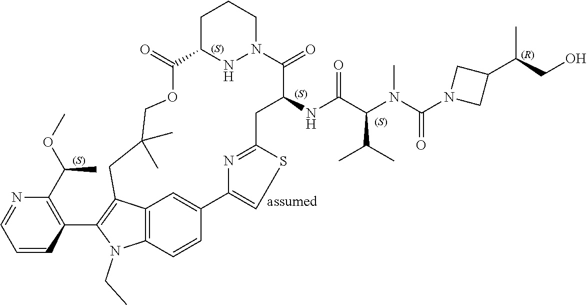 |
| A488 | 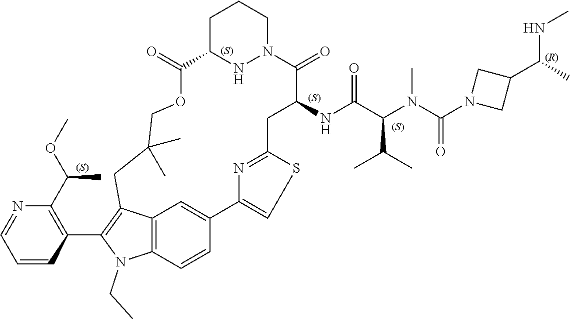 |
| A489 | 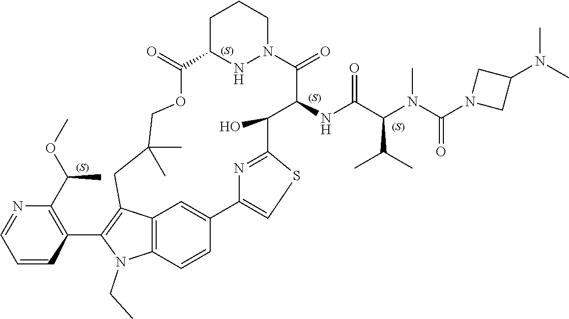 |
| A490 | 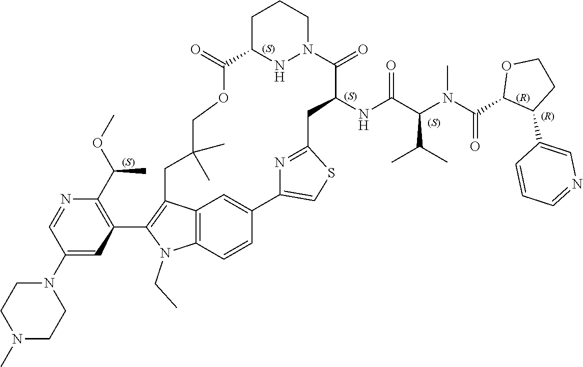 |
| A491 | 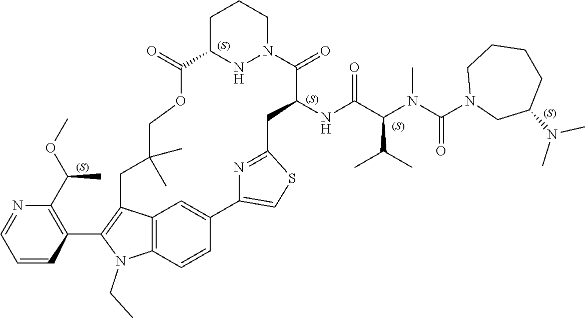 |
| A492 | 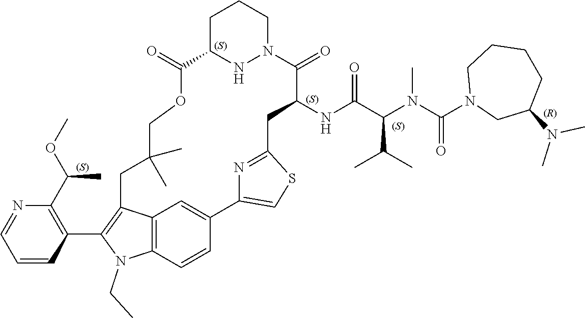 |
| A493 | 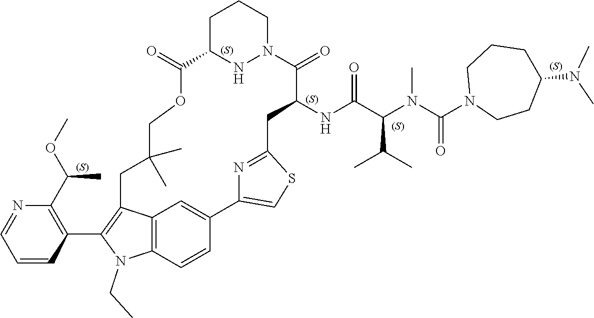 |
| A494 | 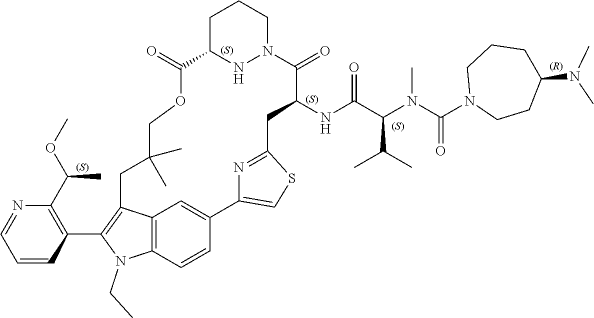 |
| A495 | 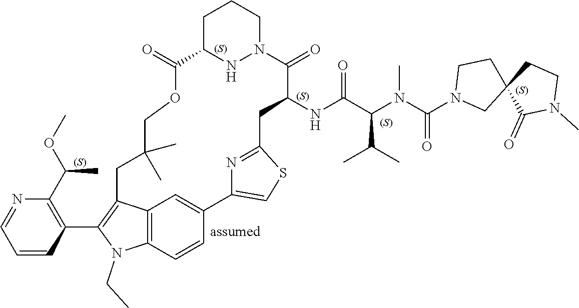 |
| A496 | 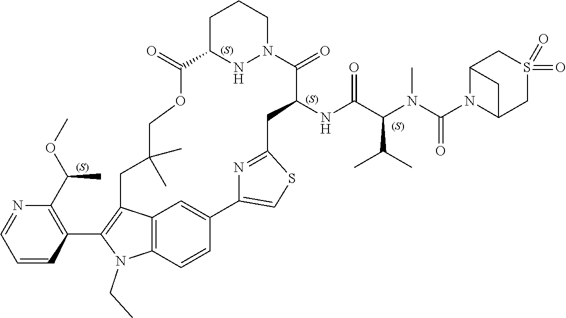 |
| A497 | 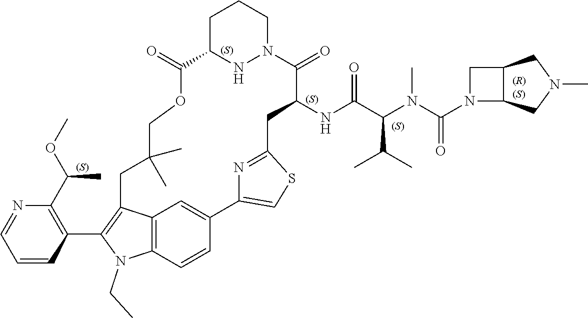 |
| A498 | 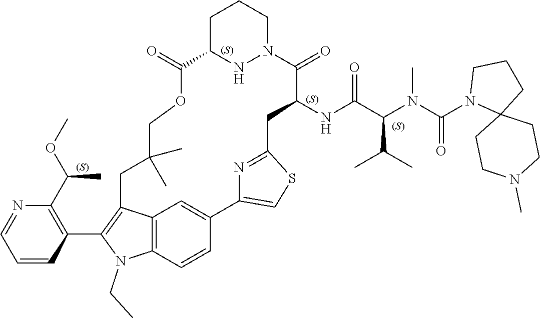 |
| A499 | 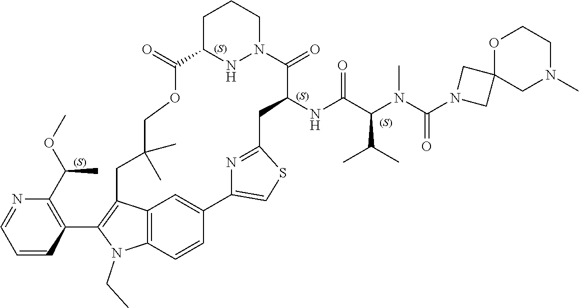 |
| A500 | 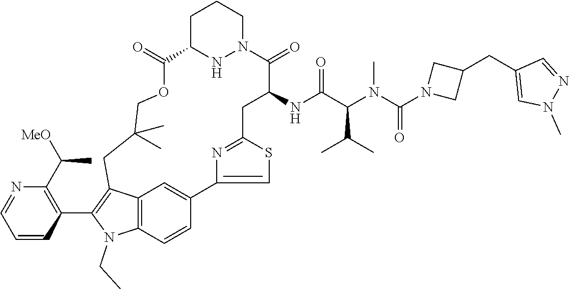 |
| A501 | 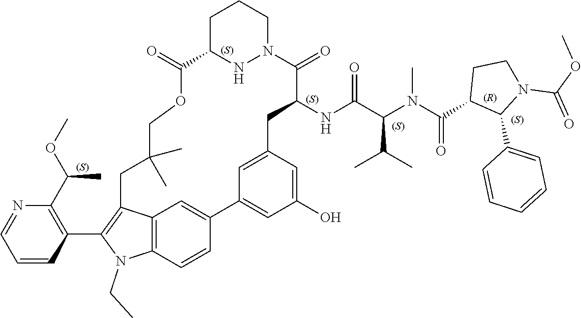 |
| A502 | 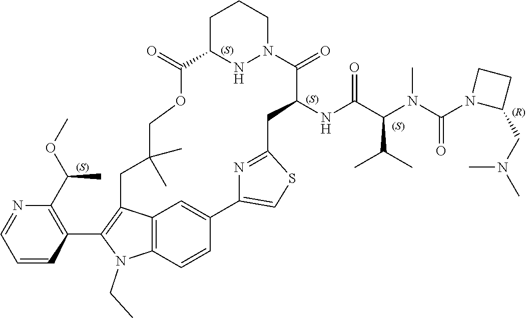 |
| A503 | 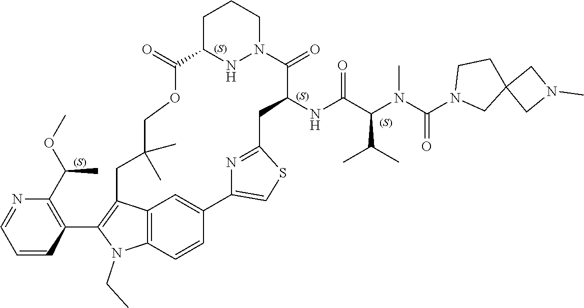 |
| A504 | 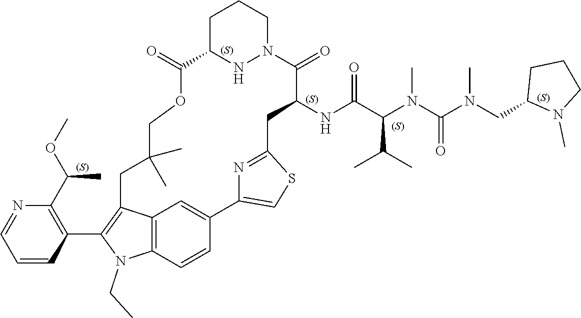 |
| A505 | 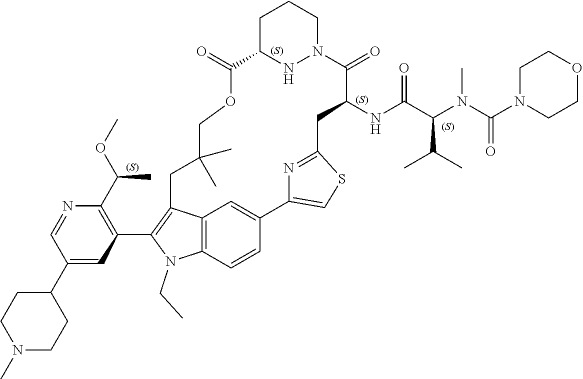 |
| A506 | 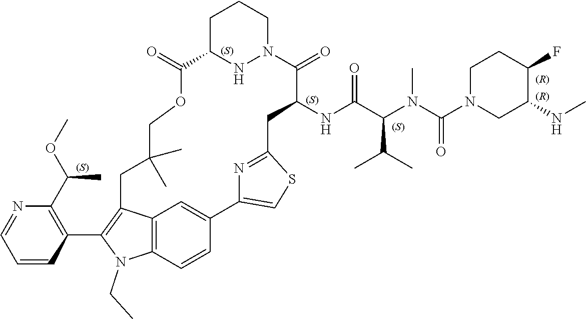 |
| A507 | 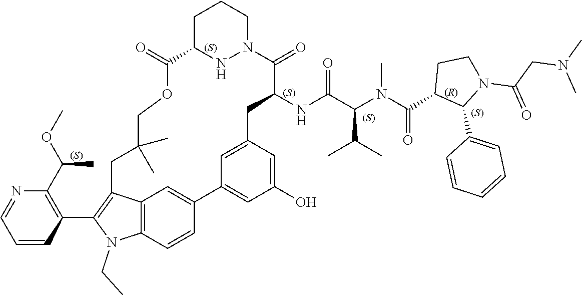 |
| A508 | 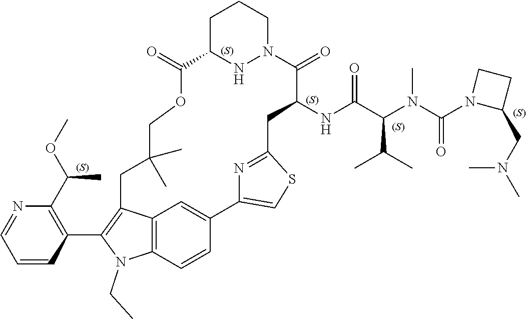 |
| A509 | 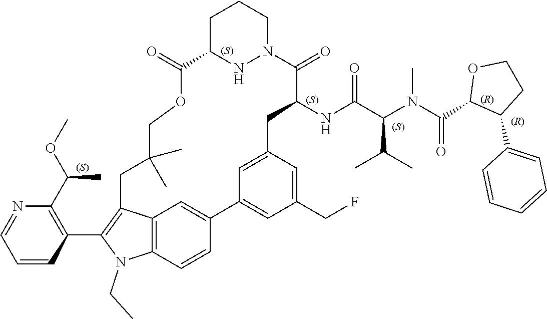 |
| A510 | 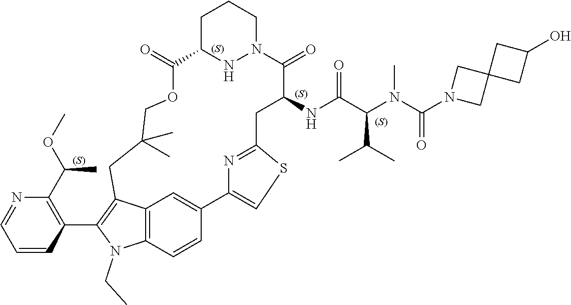 |
| A511 | 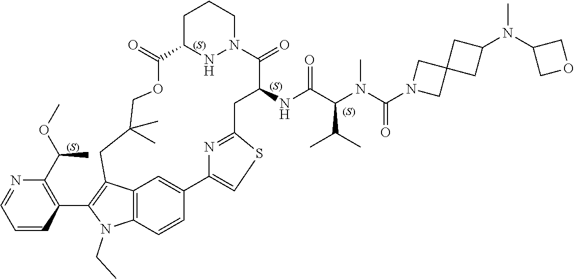 |
| A512 | 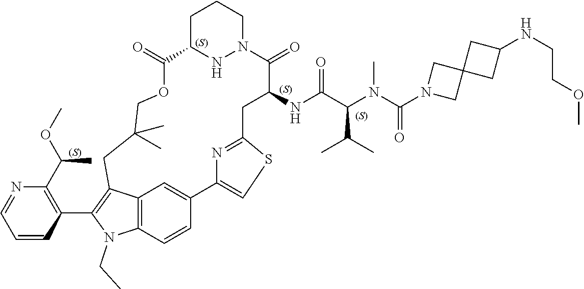 |
| A513 | 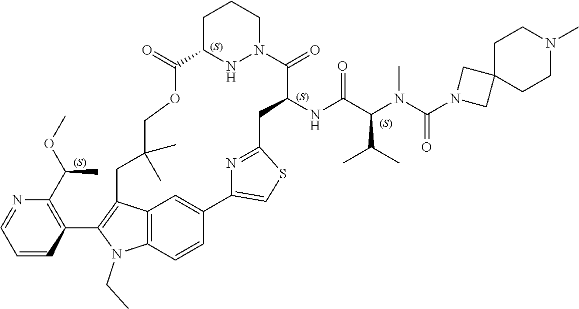 |
| A514 | 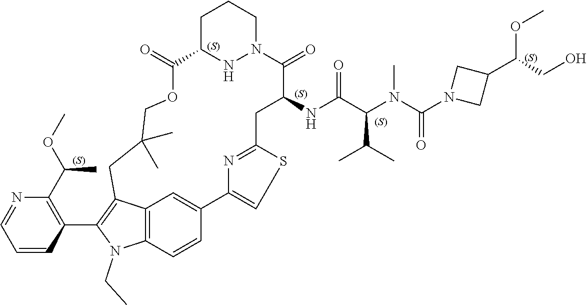 |
| A515 | 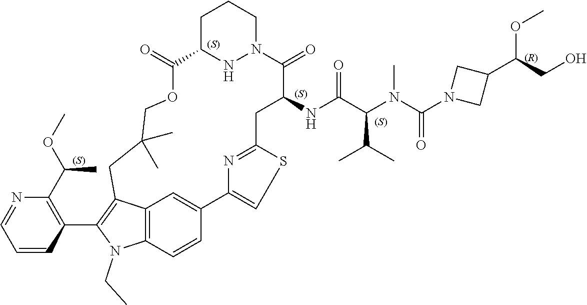 |
| A516 | 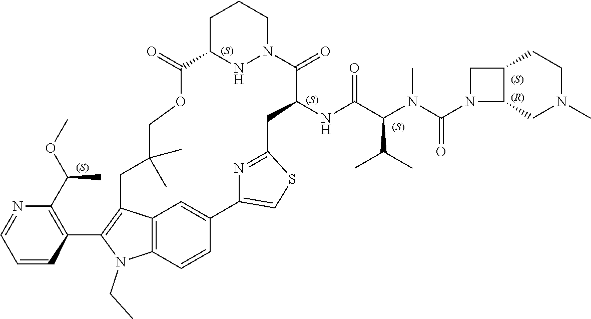 |
| A517 | 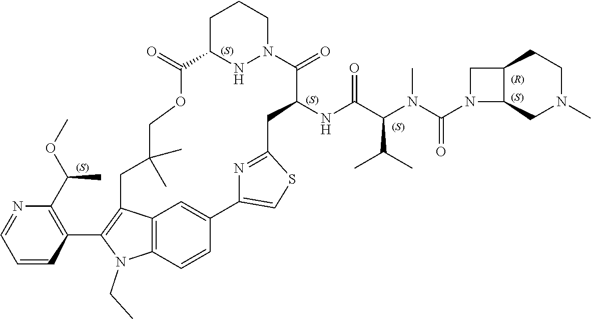 |
| A518 | 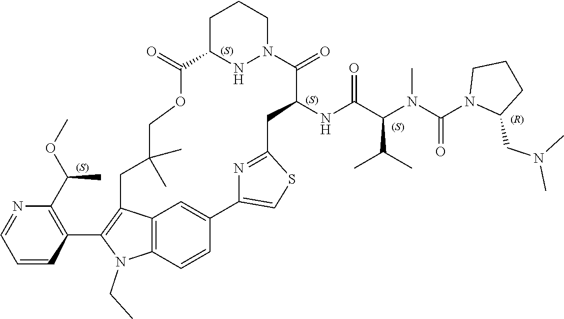 |
| A519 | 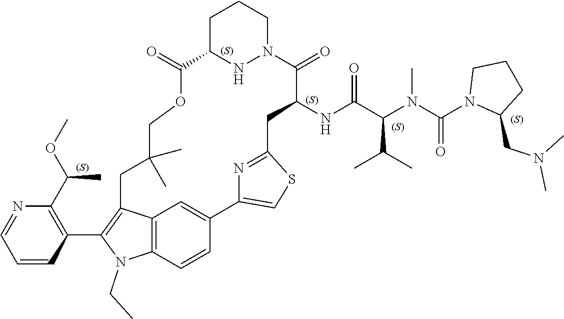 |
| A520 | 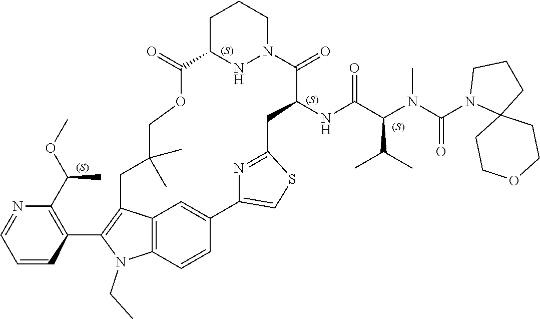 |
| A521 | 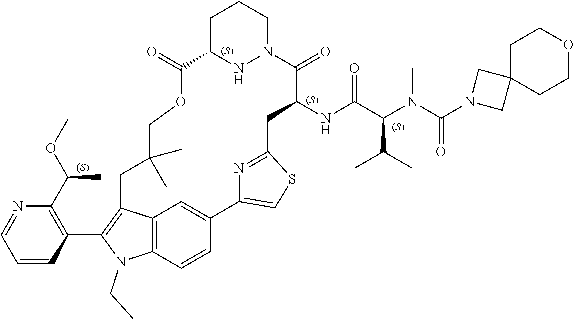 |
| A522 | 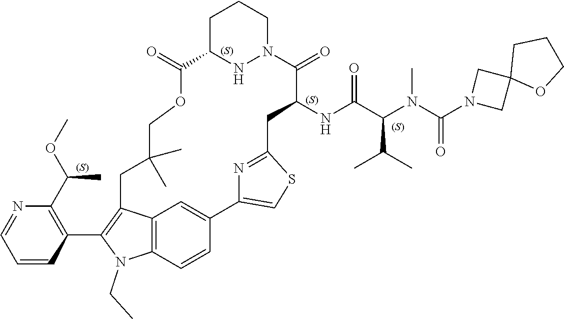 |
| A523 | 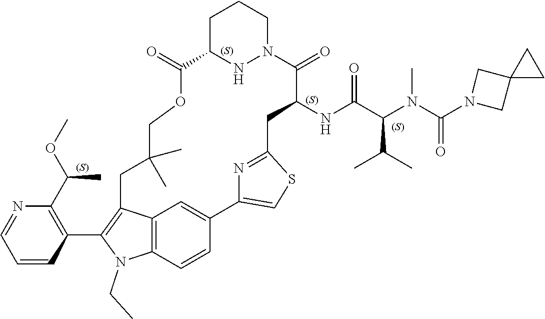 |
| A524 | 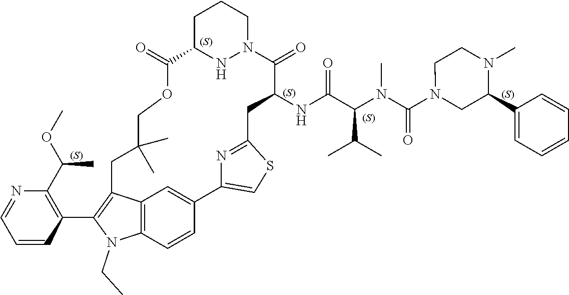 |
| A525 | 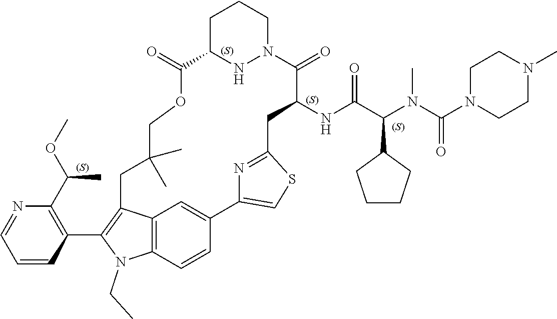 |
| A526 | 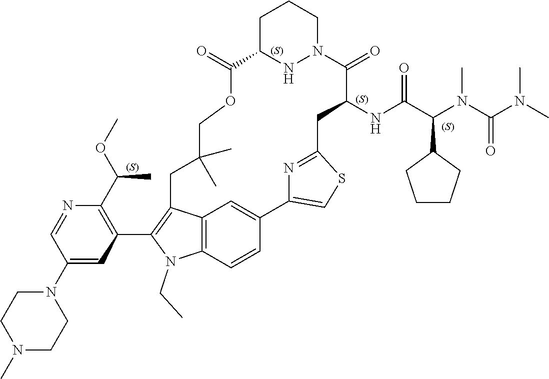 |
| A527 | 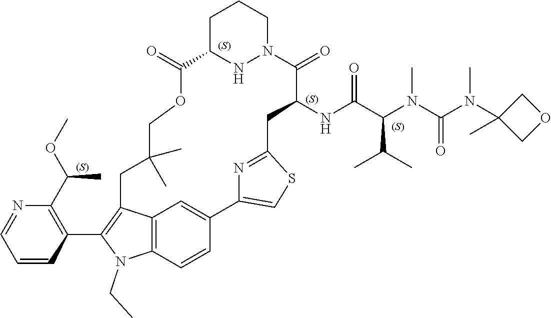 |
| A528 | 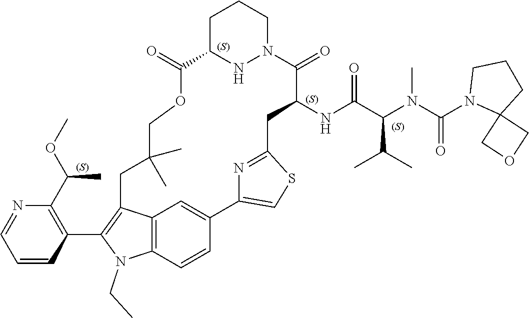 |
| A529 | 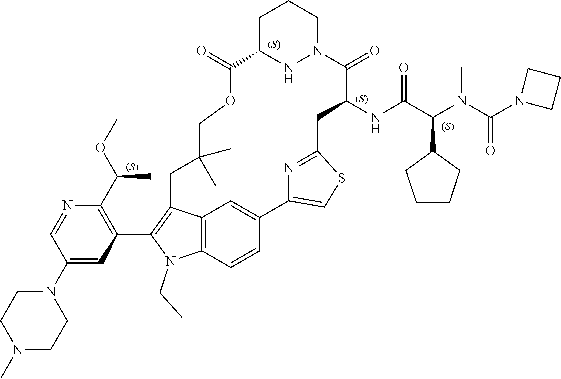 |
| A530 | 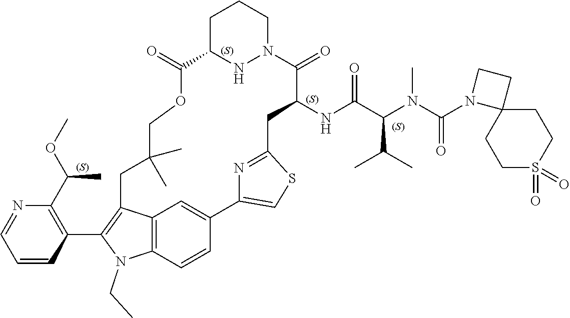 |
| A531 | 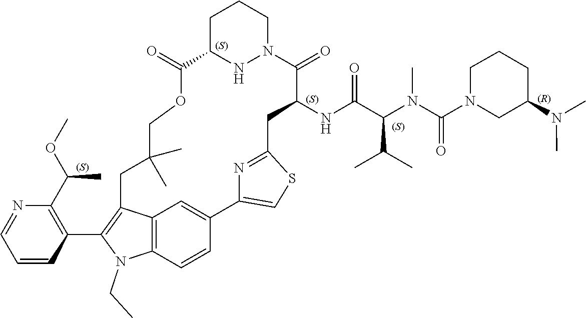 |
| A532 | 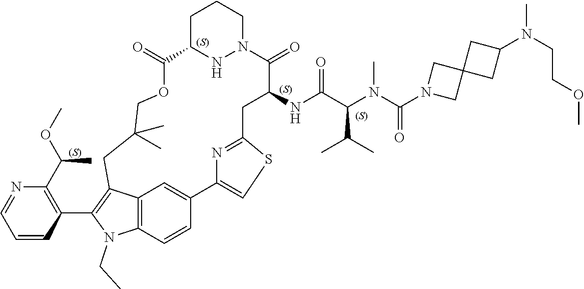 |
| A533 | 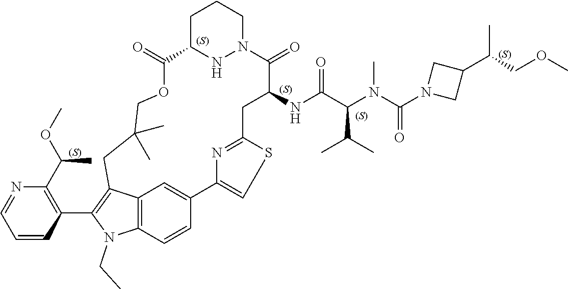 |
| A534 | 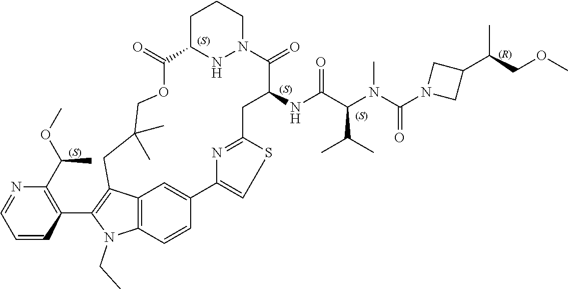 |
| A535 | 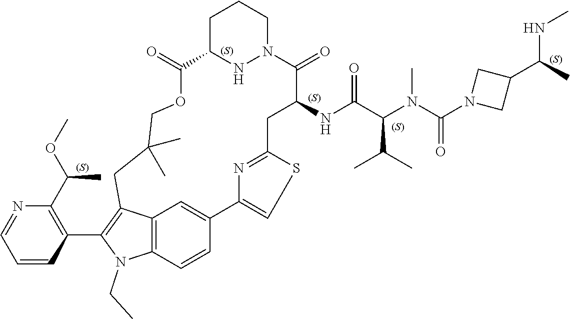 |
| A536 | 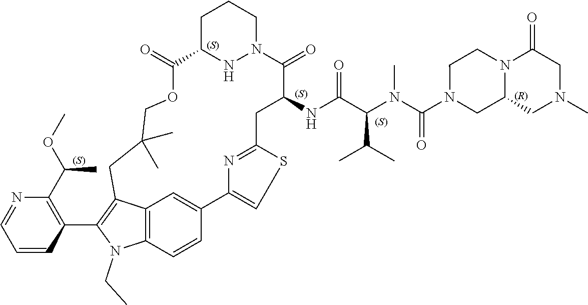 |
| A537 | 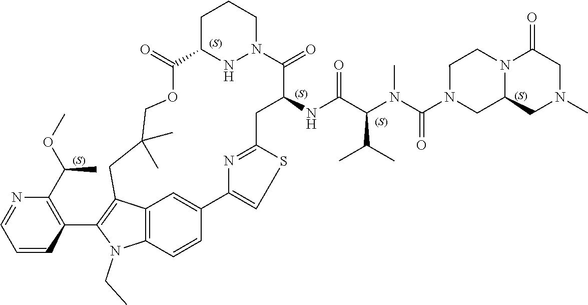 |
| A538 | 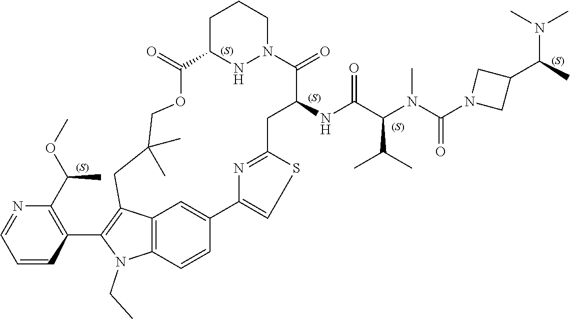 |
| A539 | 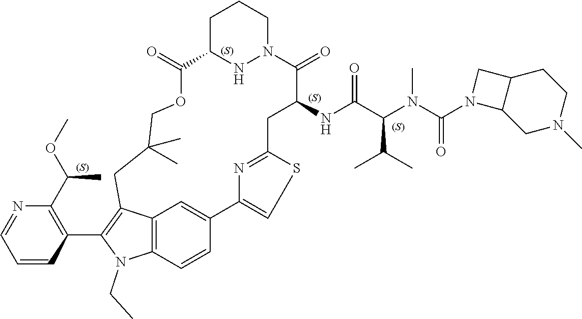 |
| A540 | 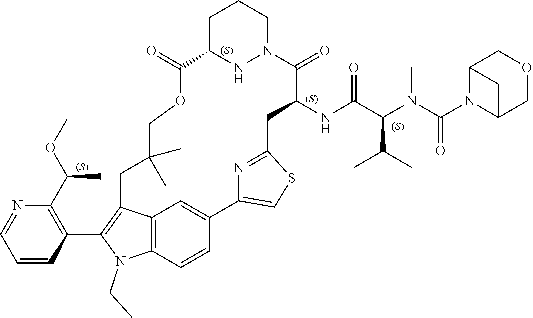 |
| A541 | 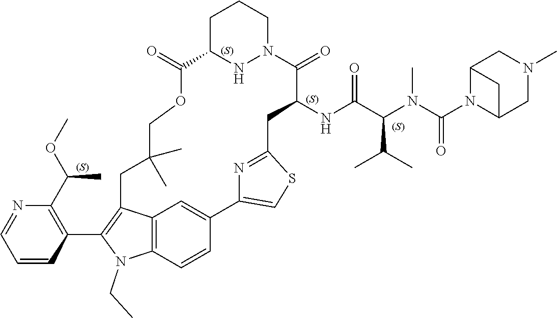 |
| A542 | 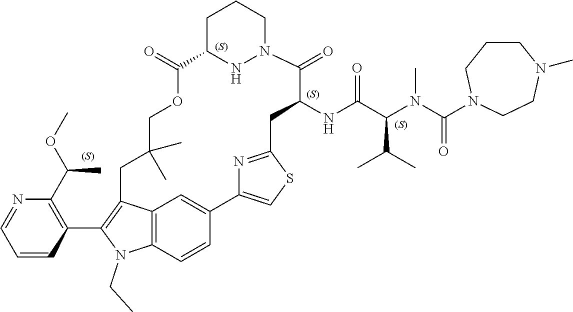 |
| A543 | 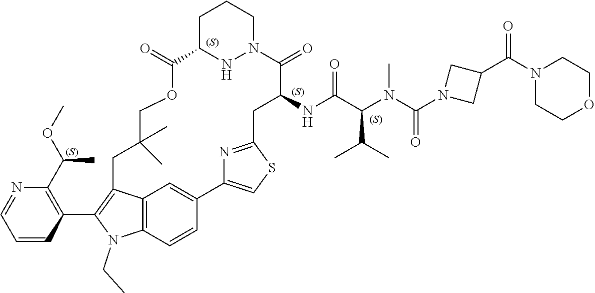 |
| A544 | 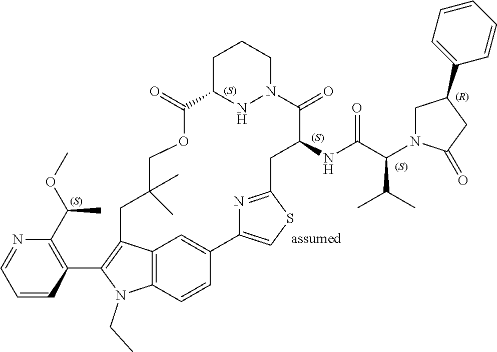 |
| A545 | 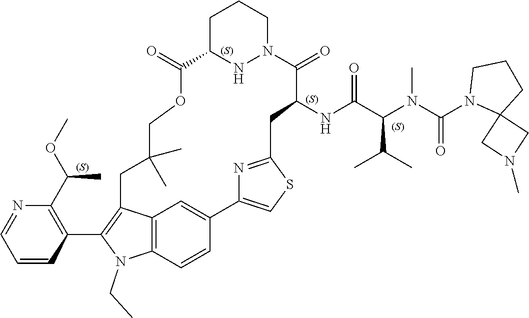 |
| A546 | 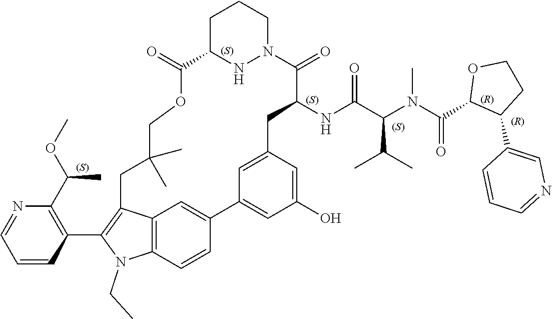 |
| A547 | 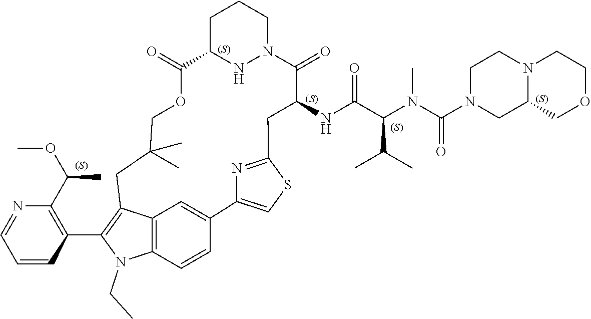 |
| A548 | 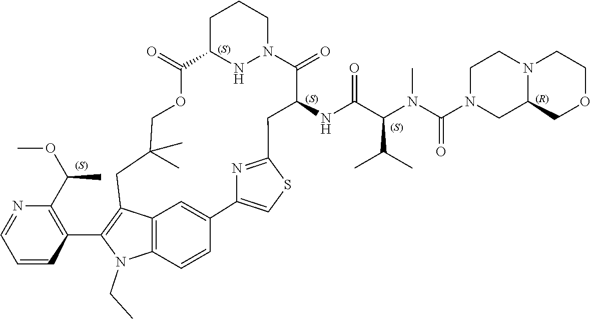 |
| A549 | 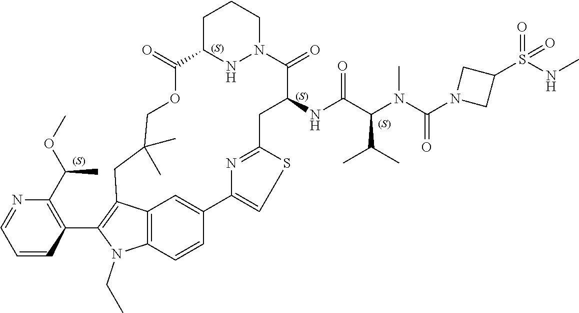 |
| A550 | 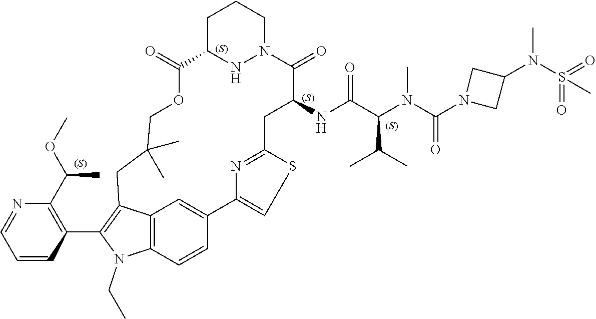 |
| A551 | 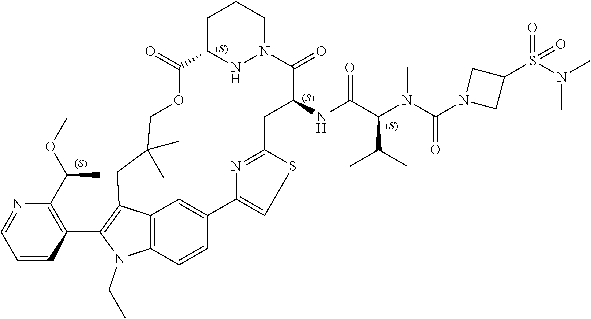 |
| A552 | 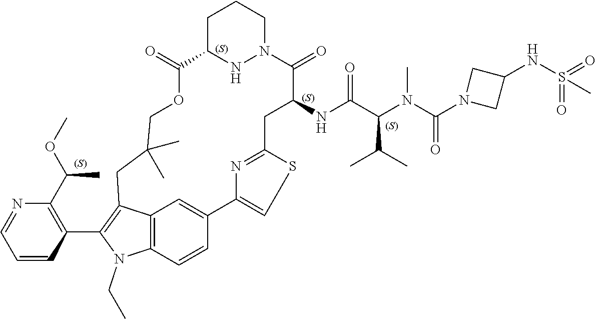 |
| A553 | 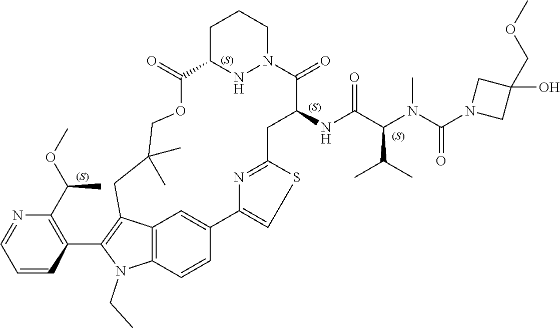 |
| A554 | 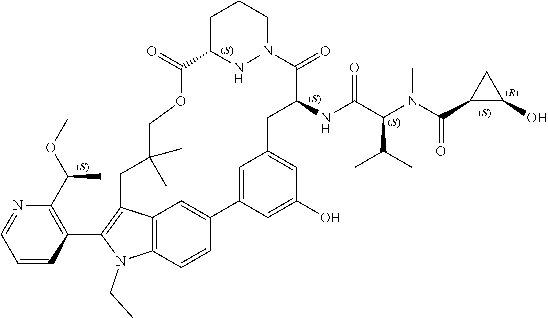 |
| A555 | 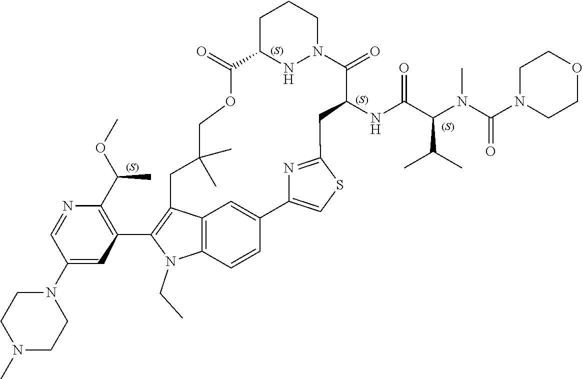 |
| A556 | 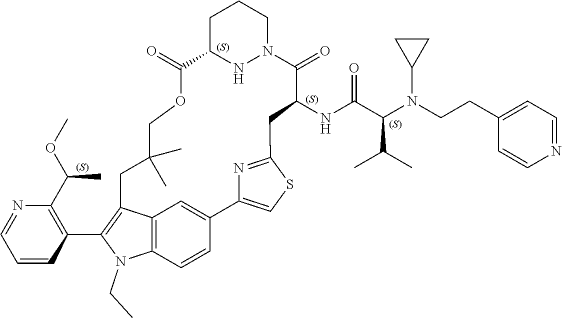 |
| A557 | 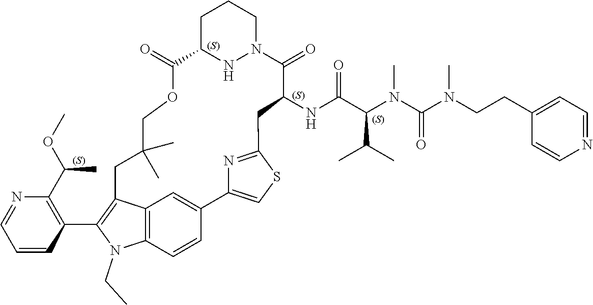 |
| A558 | 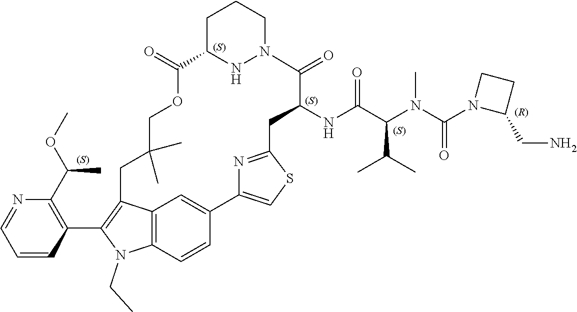 |
| A559 | 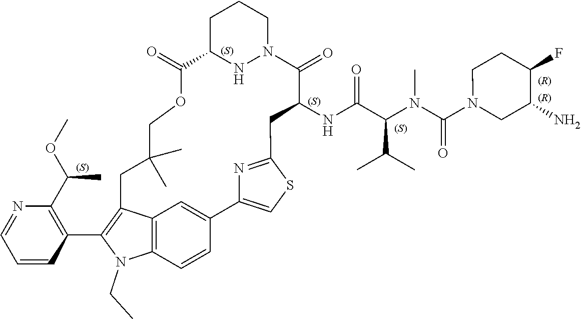 |
| A560 | 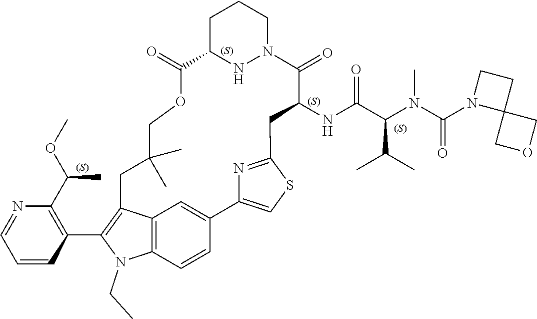 |
| A561 | 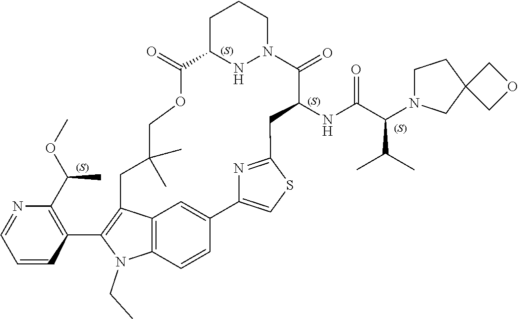 |
| A562 | 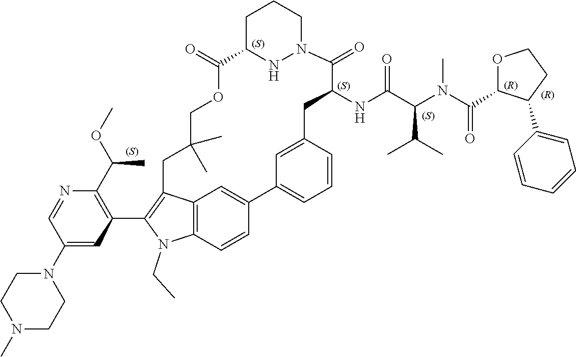 |
| A563 | 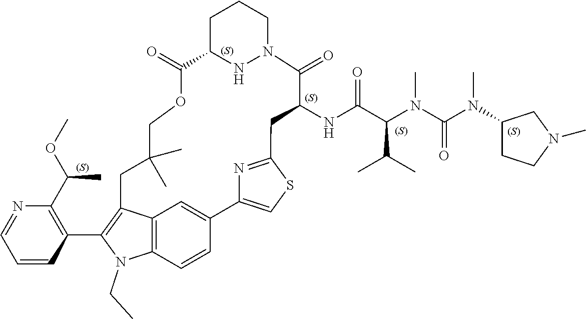 |
| A564 | 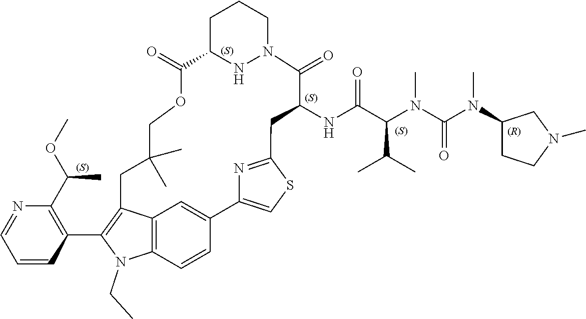 |
| A565 | 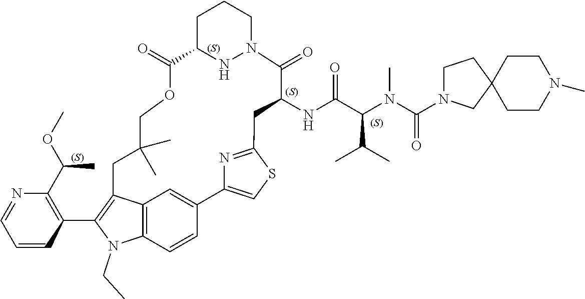 |
| A566 | 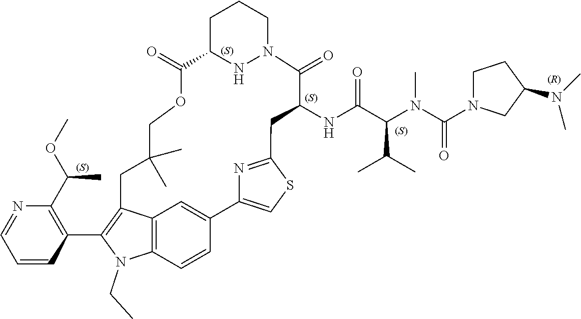 |
| A567 | 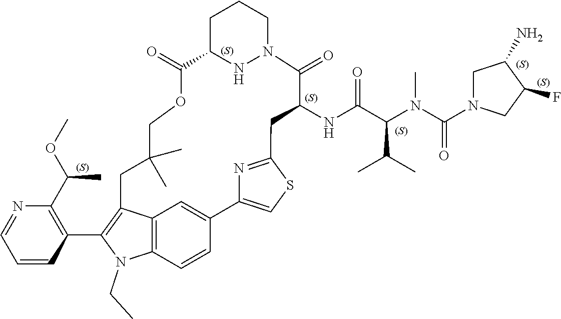 |
| A568 | 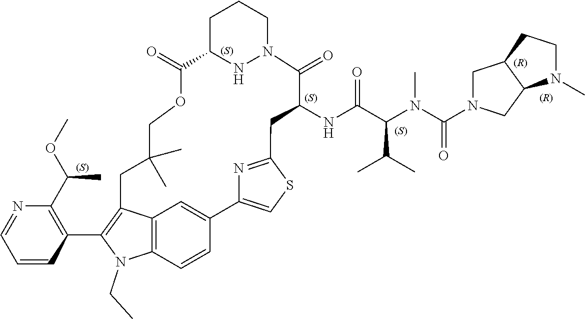 |
| A569 | 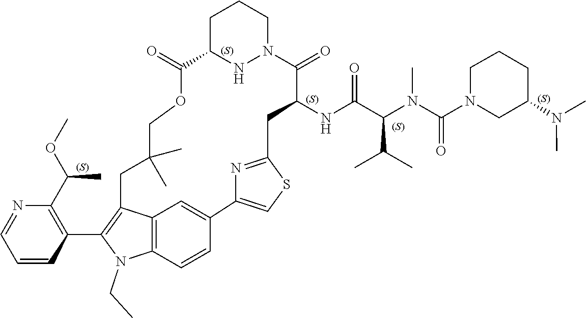 |
| A570 | 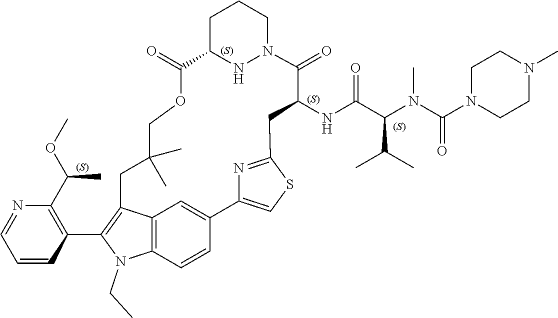 |
| A571 | 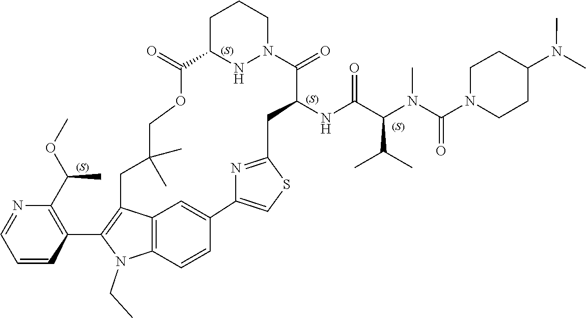 |
| A572 | 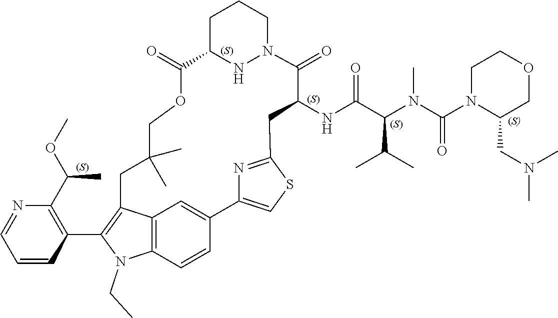 |
| A573 | 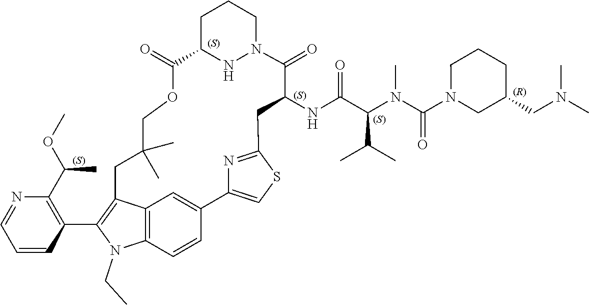 |
| A574 | 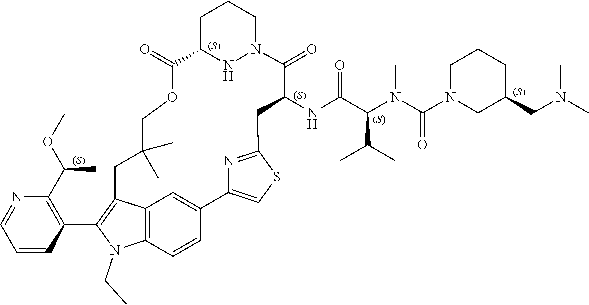 |
| A575 | 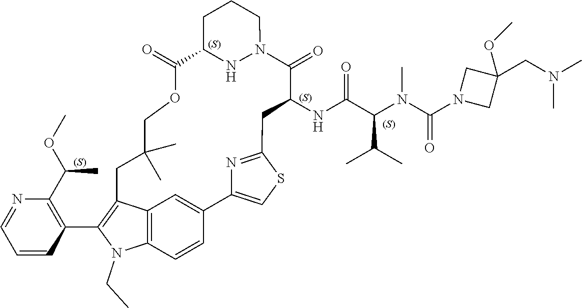 |
| A576 | 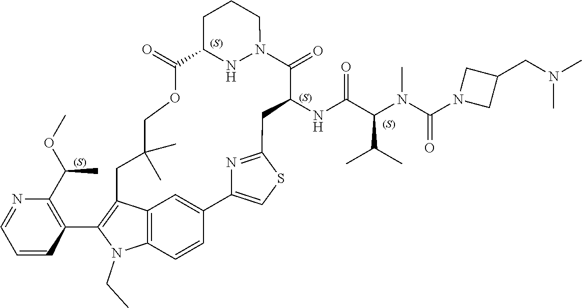 |
| A577 | 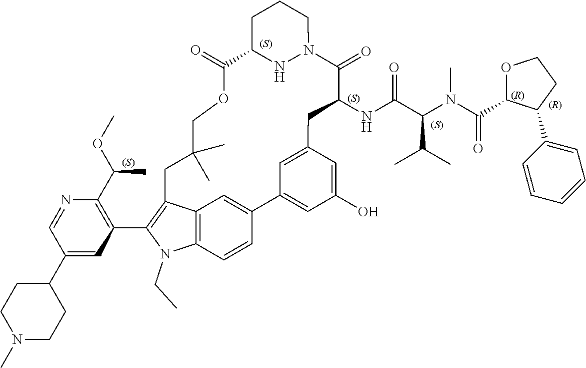 |
| A578 | 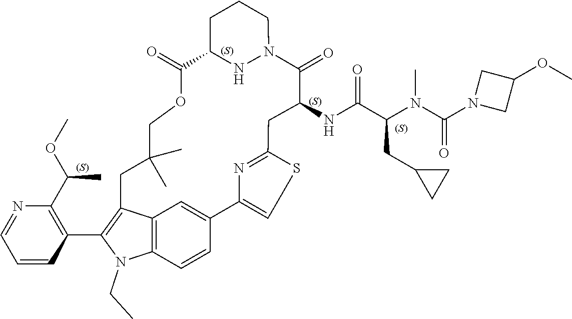 |
| A579 | 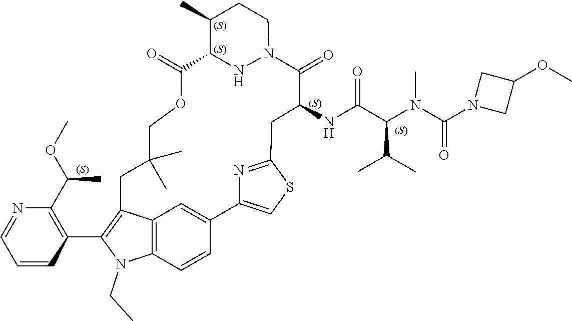 |
| A580 | 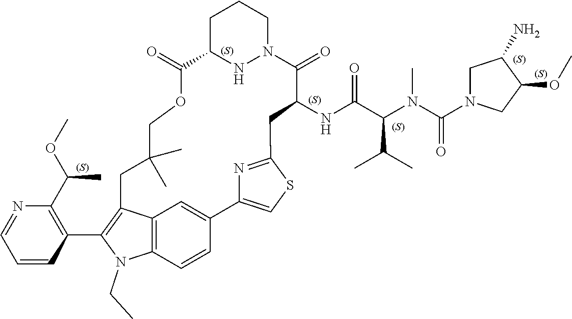 |
| A581 | 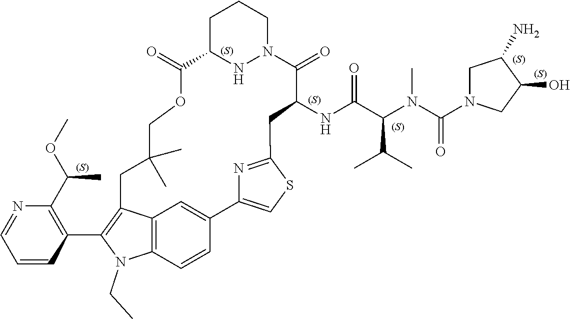 |
| A582 | 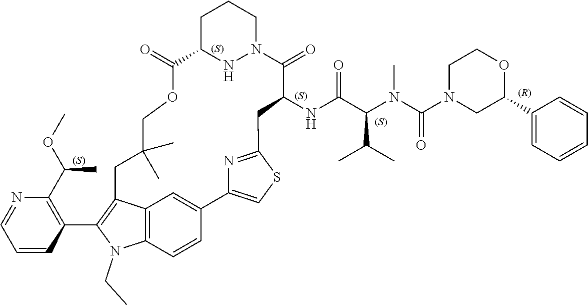 |
| A583 | 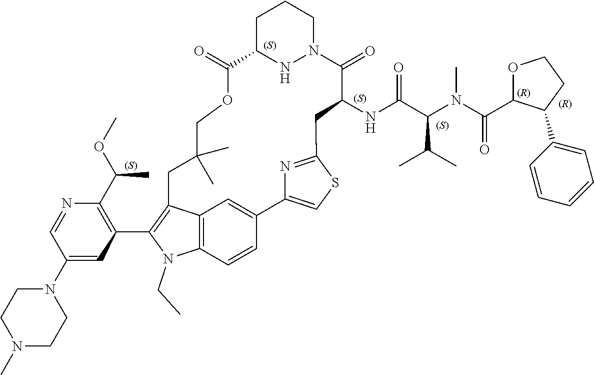 |
| A584 | 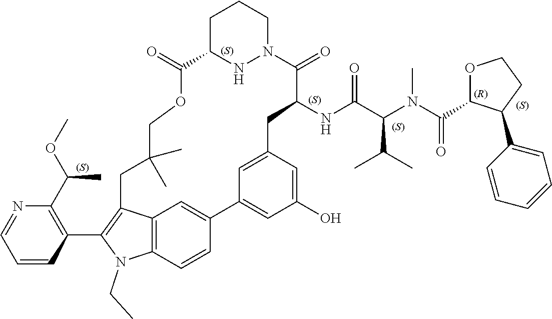 |
| A585 | 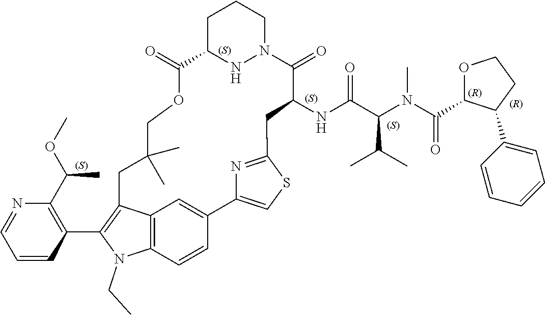 |
| A586 | 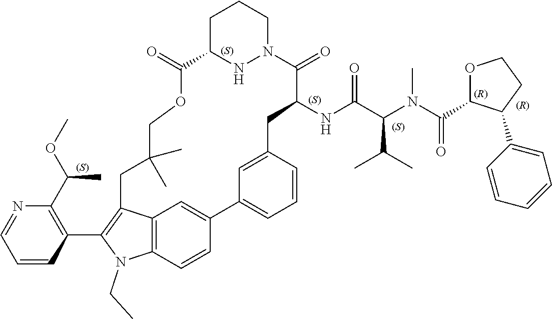 |
| A587 | 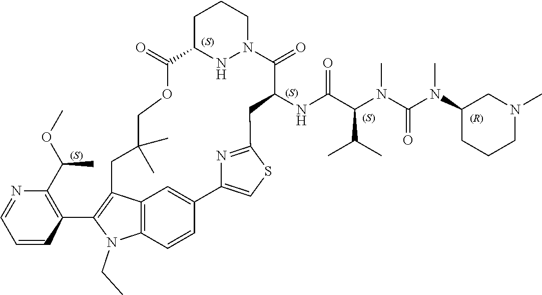 |
| A588 | 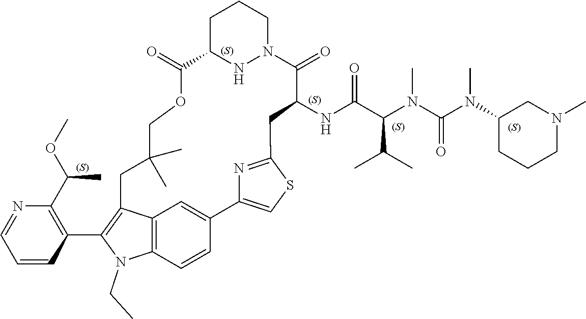 |
| A589 | 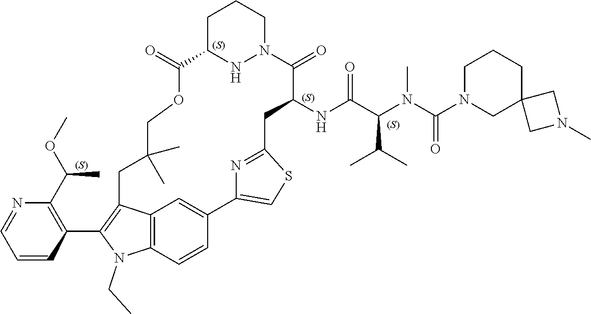 |
| A590 | 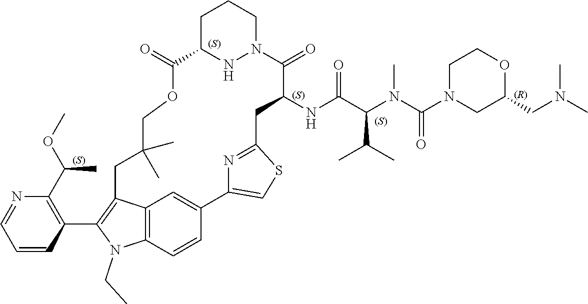 |
| A591 | 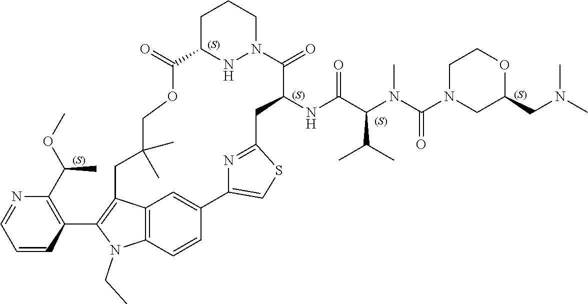 |
| A592 | 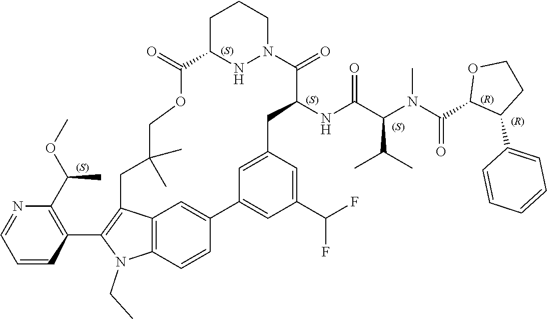 |
| A593 | 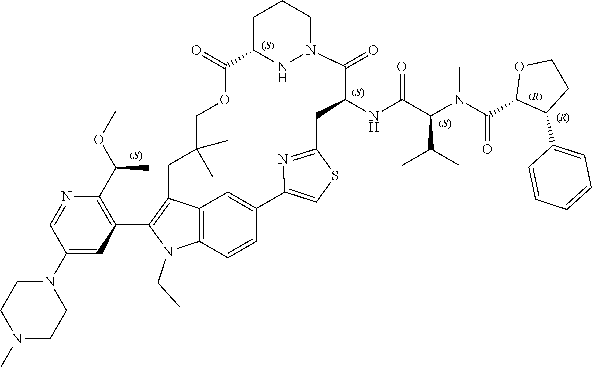 |
| A594 | 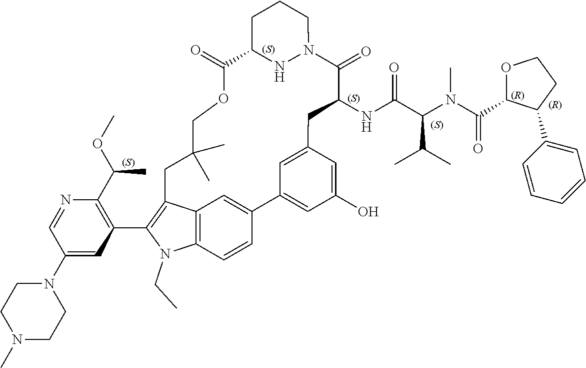 |
| A595 | 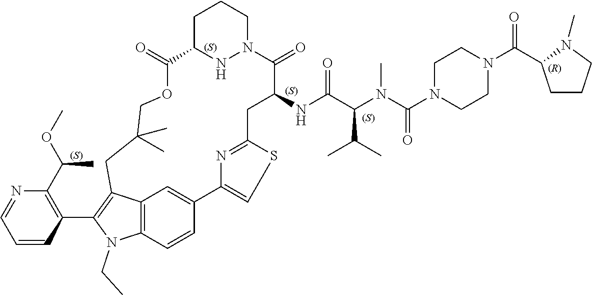 |
| A596 | 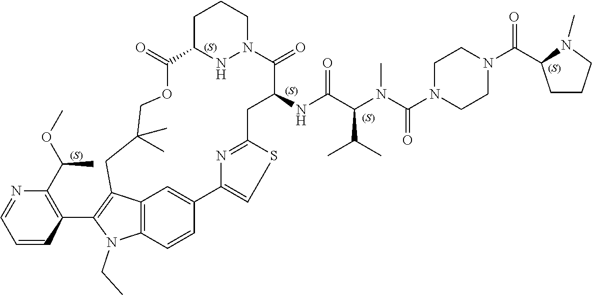 |
| A597 | 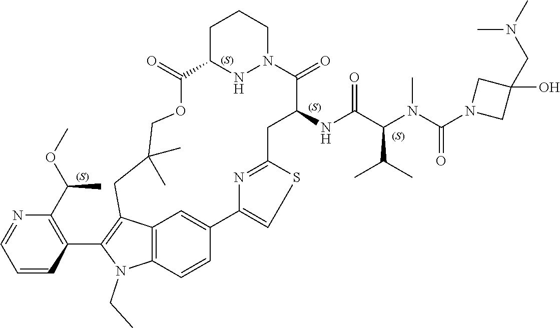 |
| A598 | 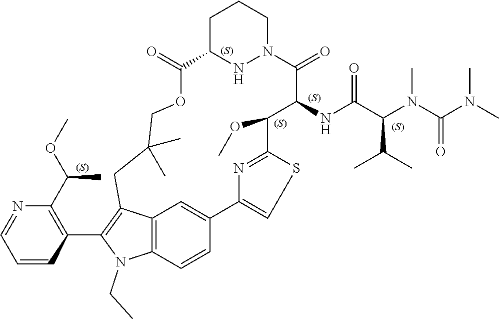 |
| A599 | 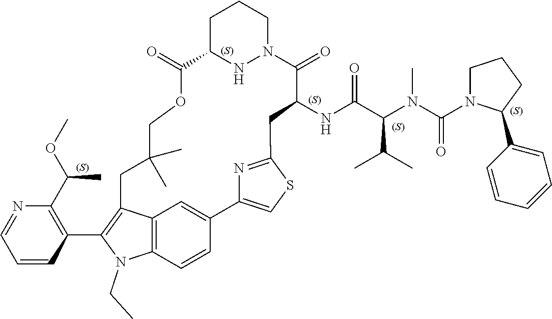 |
| A600 | 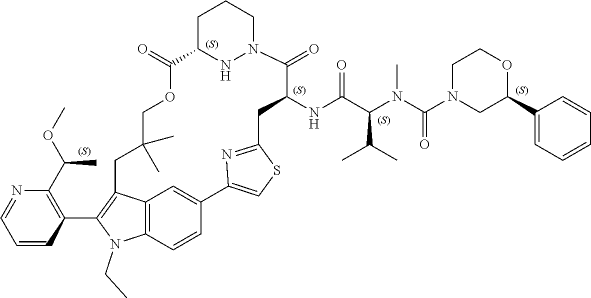 |
| A601 | 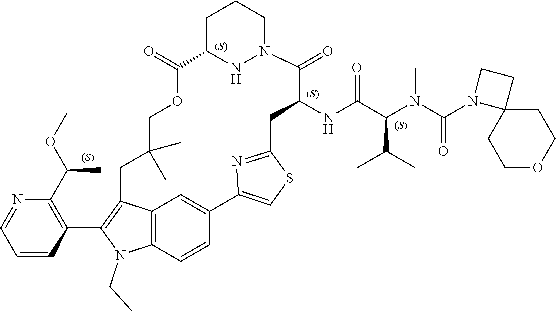 |
| A602 | 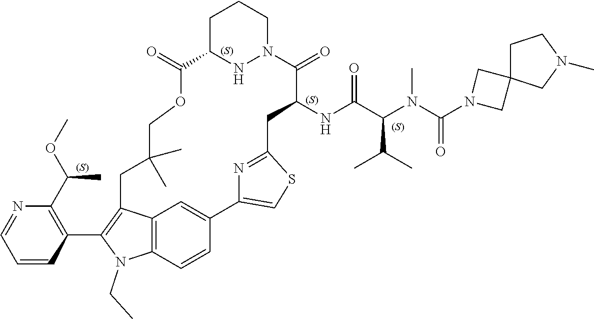 |
| A603 | 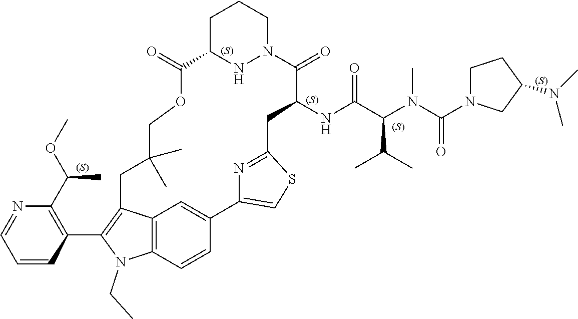 |
| A604 | 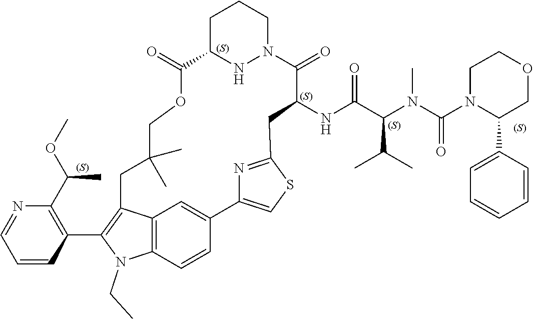 |
| A605 | 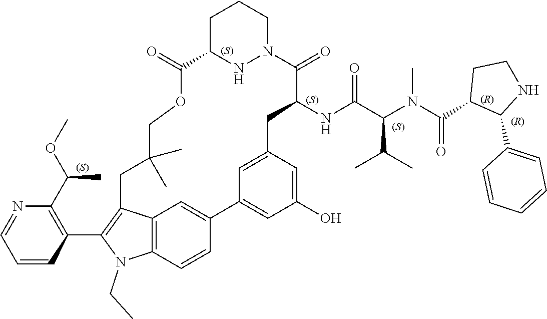 |
| A606 | 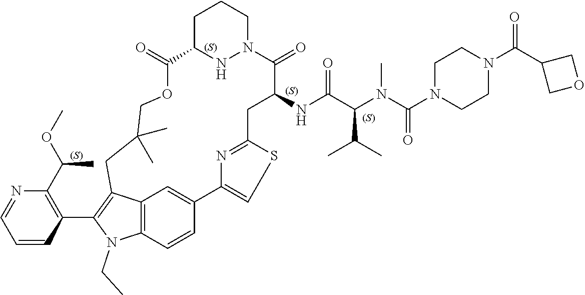 |
| A607 | 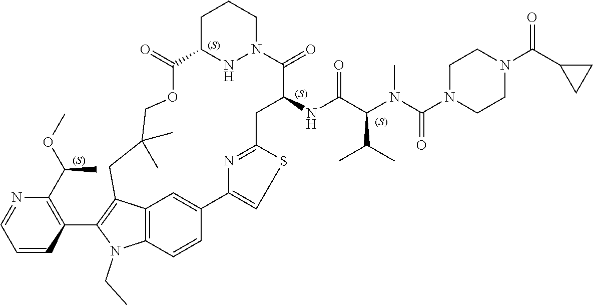 |
| A608 | 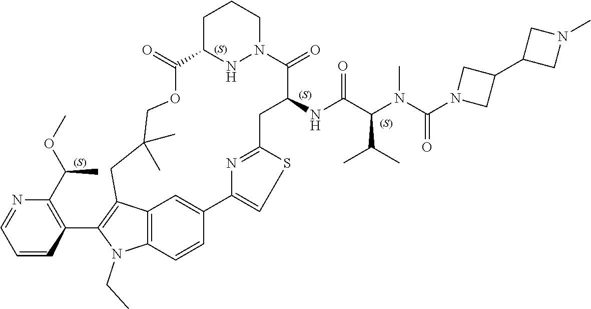 |
| A609 | 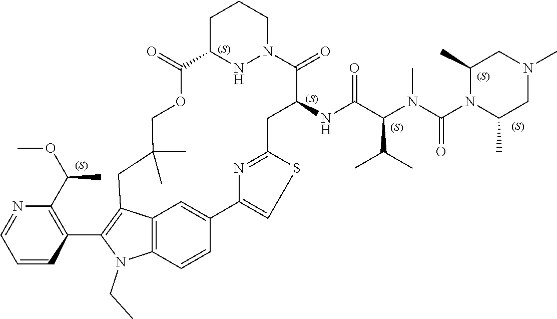 |
| A610 | 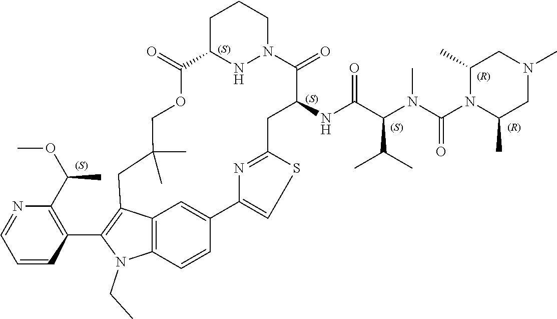 |
| A611 | 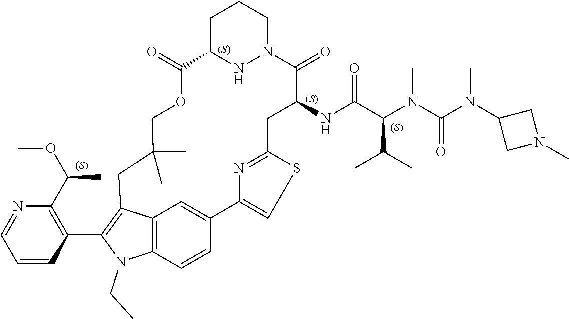 |
| A612 | 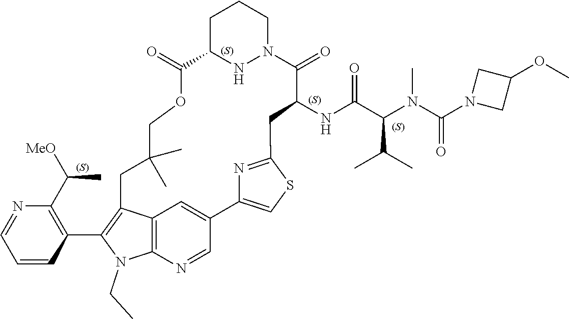 |
| A613 | 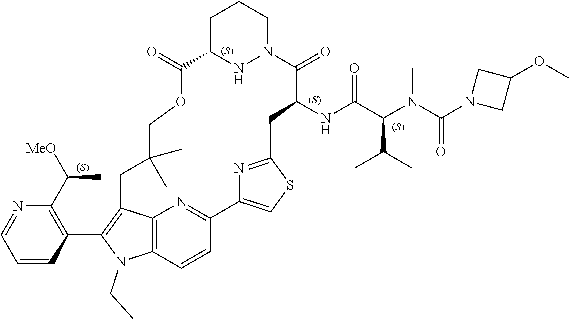 |
| A614 | 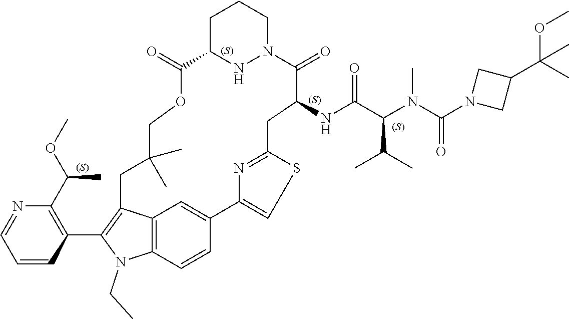 |
| A615 | 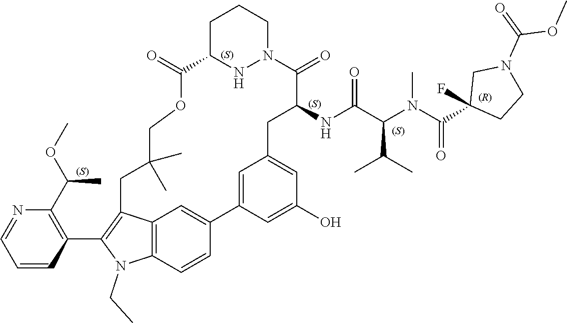 |
| A616 | 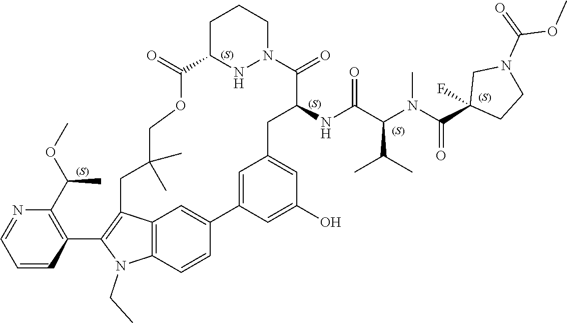 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Any compound shown in brackets indicates that the compound is a disastereomer, and the absolute stereochemistry of such diastereomer may not be known.

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B4 | 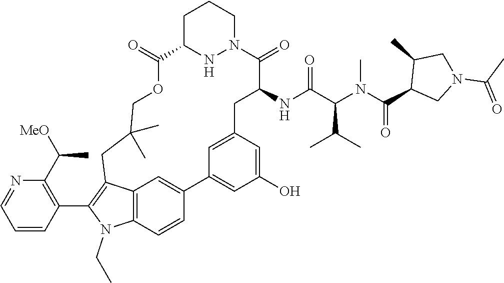 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| B5 | 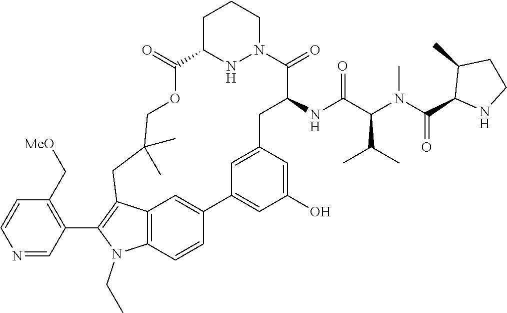 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| B6 | 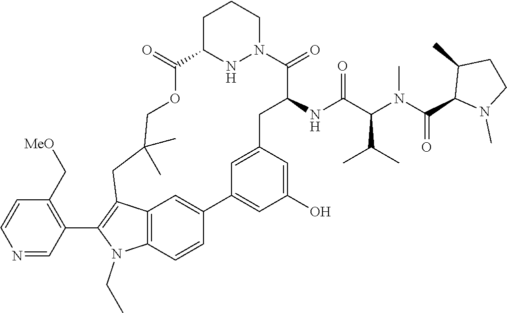 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| B8 | 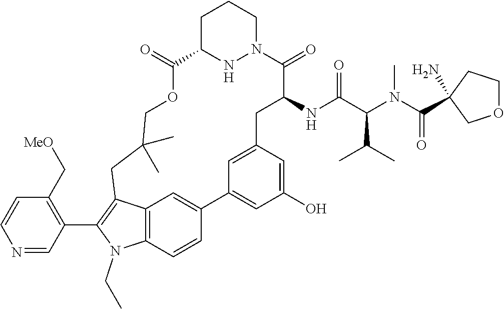 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| B9 | 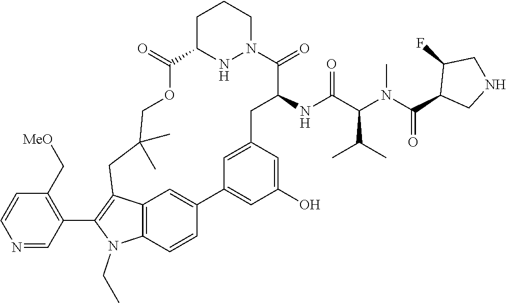 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| B12 | 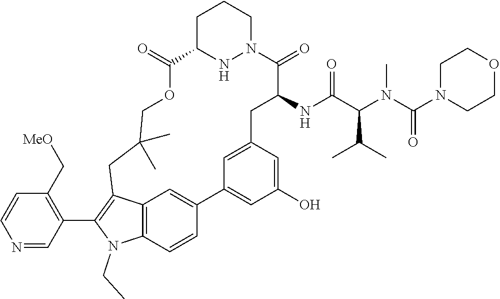 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| B13 | 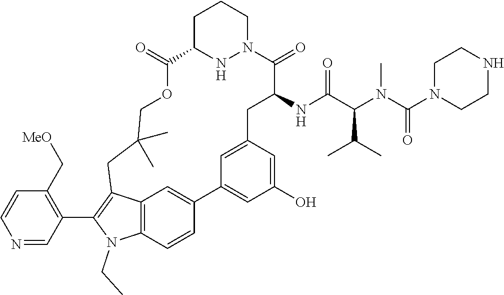 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| B19 | 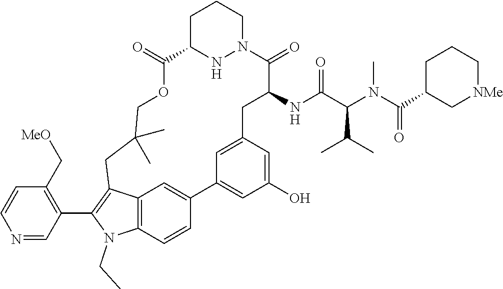 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| B44 | 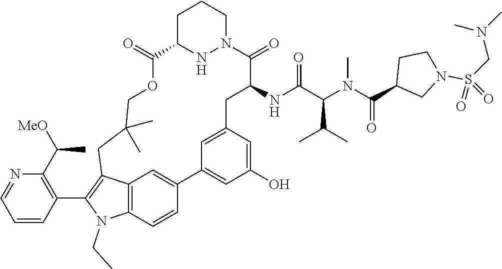 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| B47 | 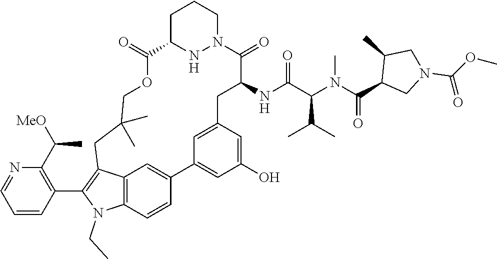 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| B57 | 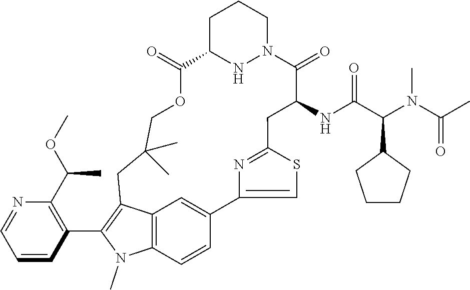 |
| B58 | 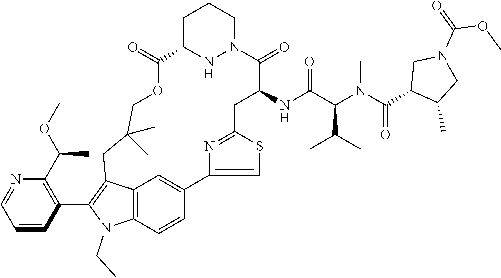 |
| B59 | 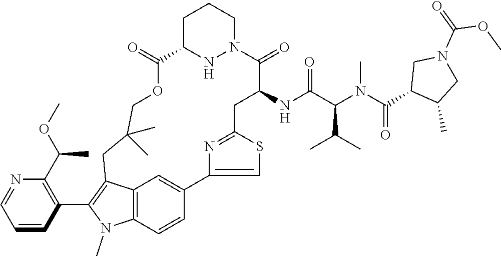 |
| B60 | 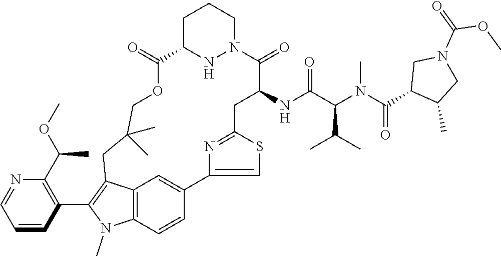 |
| B61 | 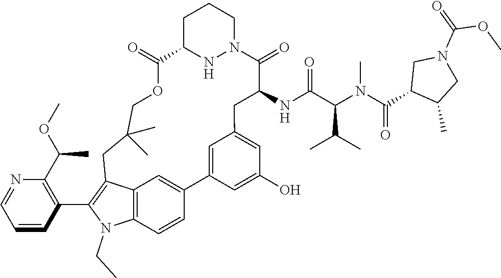 |
| B66 | 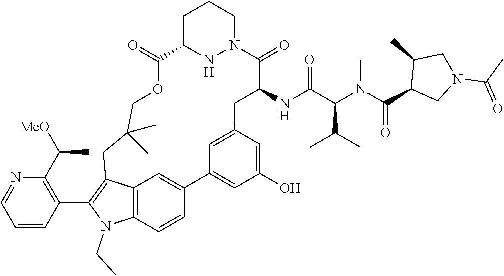 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| B67 | 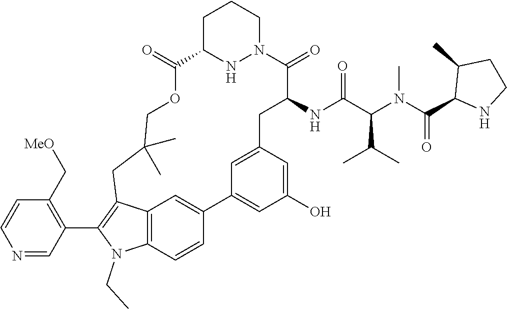 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| B69 | 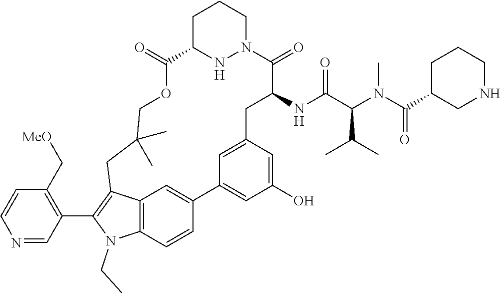 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| B71 | 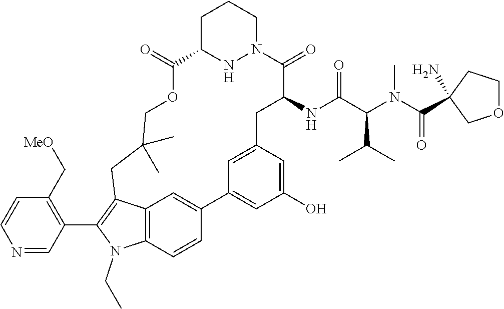 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| B73 | 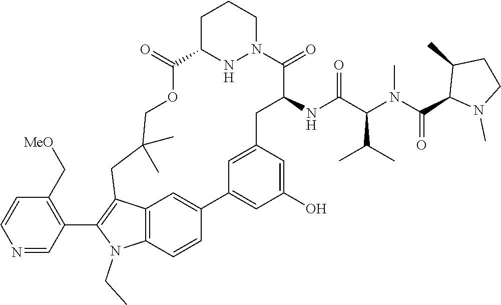 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| B74 | 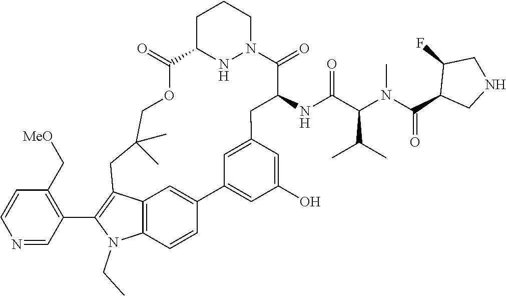 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| B80 | 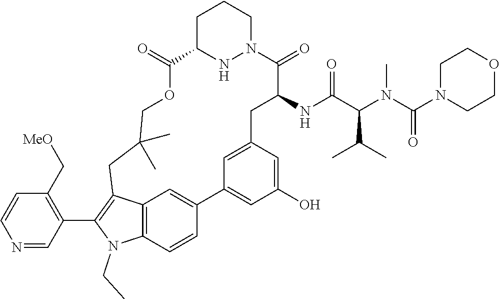 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| B81 | 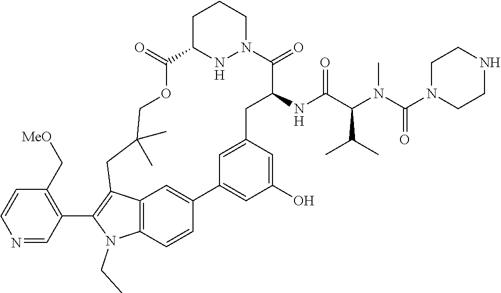 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| B94 | 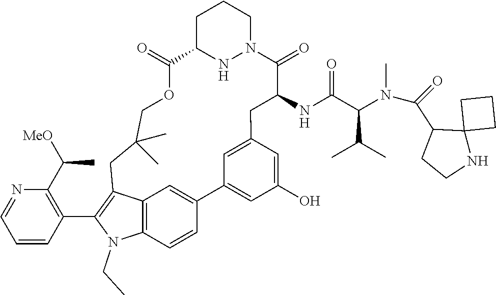 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| B95 | 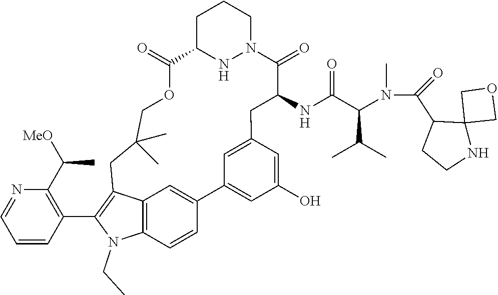 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| B96 | 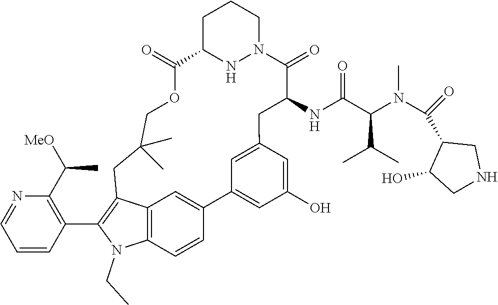 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| B97 | 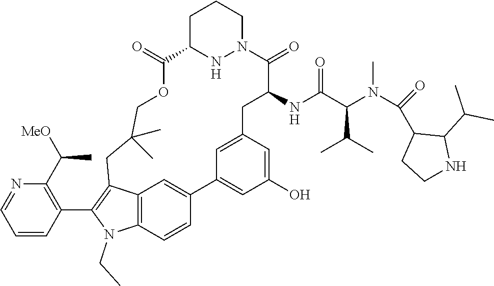 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| B99 | 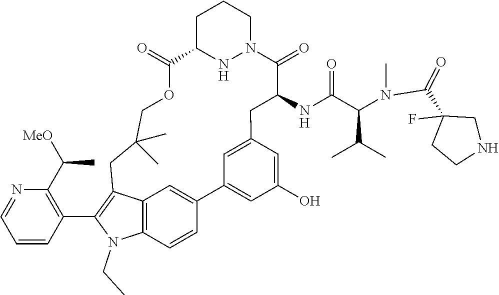 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| B100 | 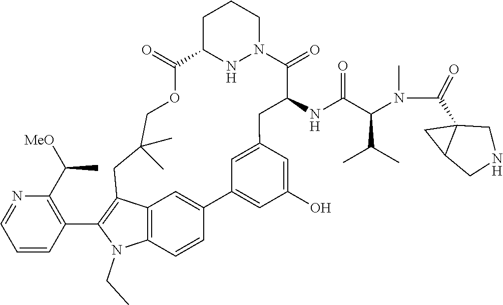 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| B104 | 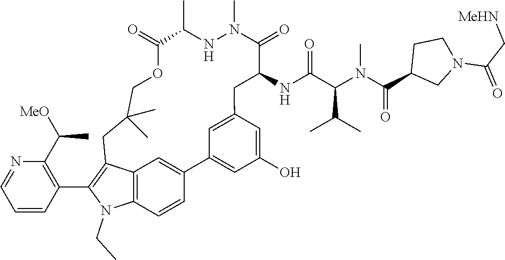 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N(C)N[C@@H](C)C(=O)OCC(C)(C)C2] |
| B106 | 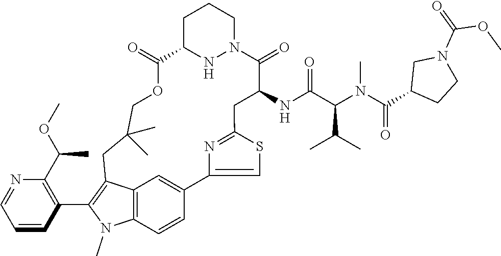 |
| B107 | 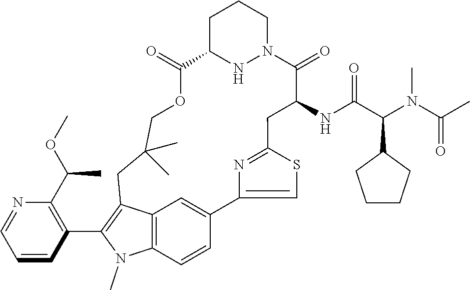 |
| B109 | 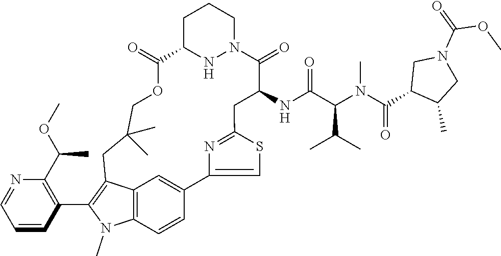 |
| B110 | 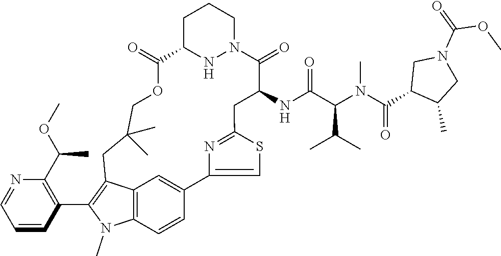 |
| B111 | 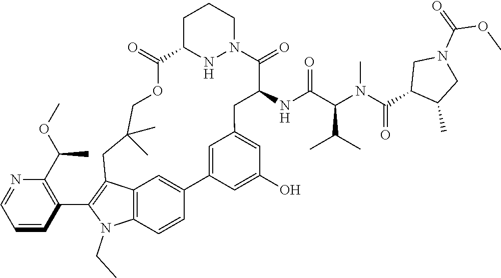 |
| B112 | 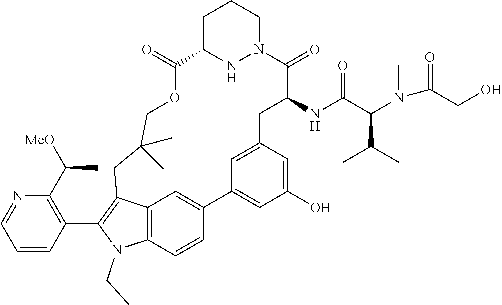 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| B113 | 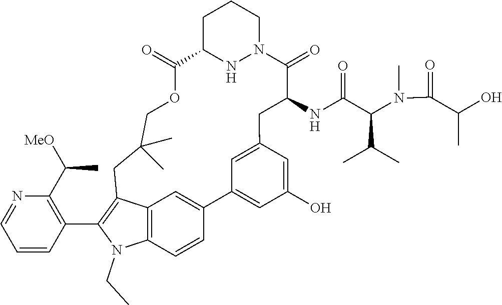 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| B114 | 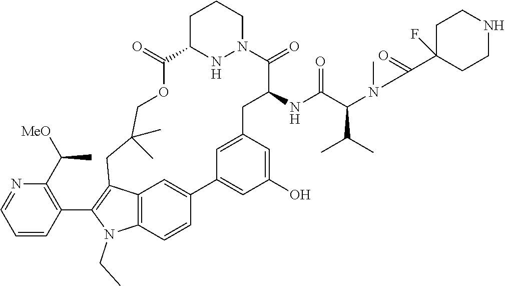 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| B117 | 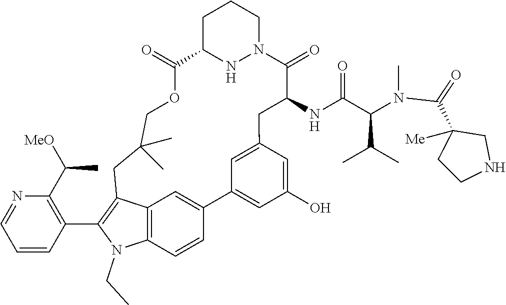 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| B119 | 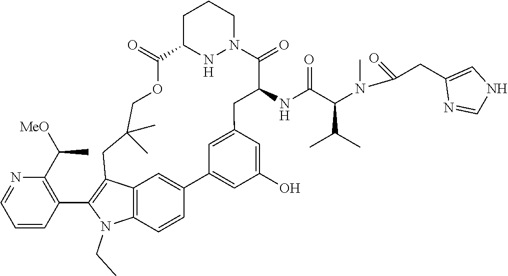 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| B122 | 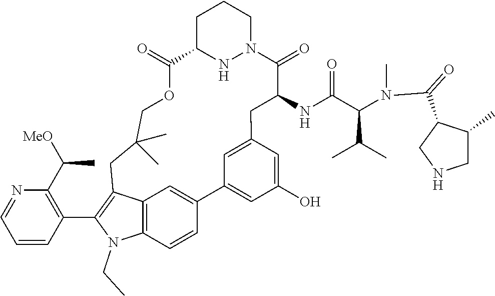 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| B123 | 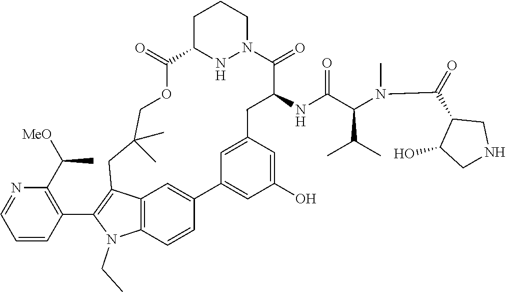 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| B124 | 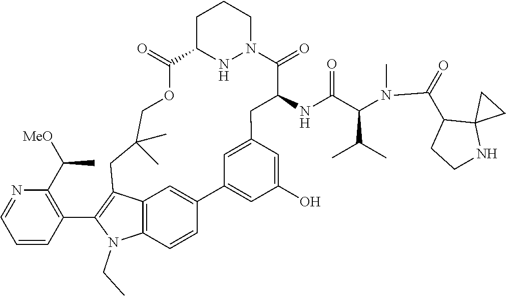 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| B126 | 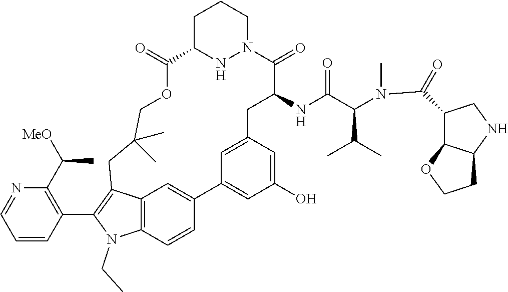 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN[C@H]3CCO[C@H]31)C(=O)N1CCC[C@H](N1)C(=O)OCC(C)(C)C2] |
| B128 | 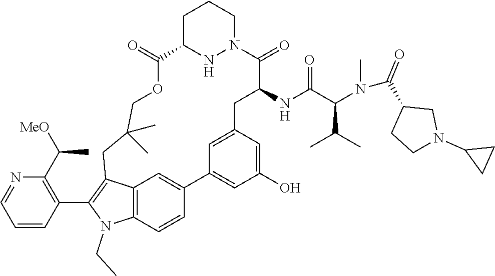 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| B129 | 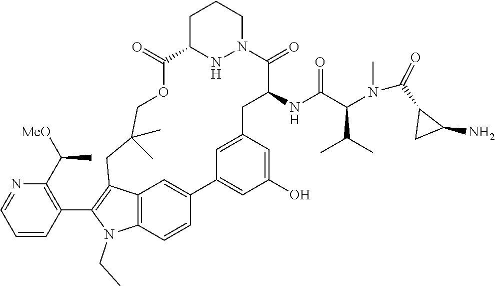 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| B130 | 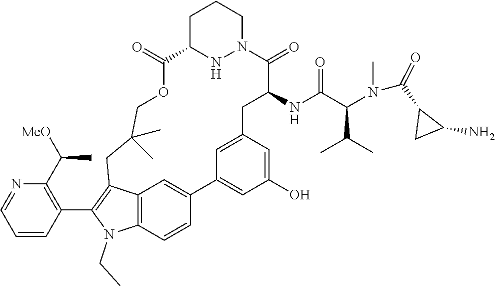 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| B133 | 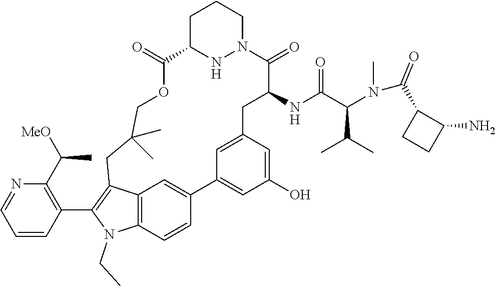 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CC[C@H]1N)C(=O)N1CCC[C@H](N1)C(=O)OCC(C)(C)C2] |
| B134 | 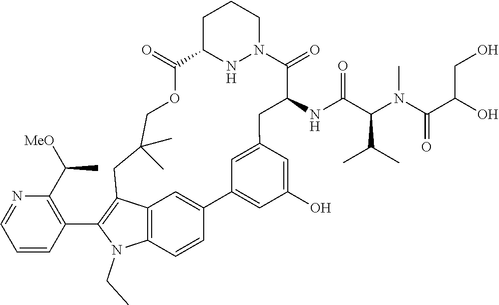 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| B135 | 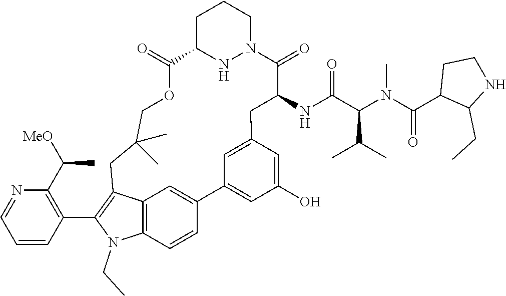 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| B137 | 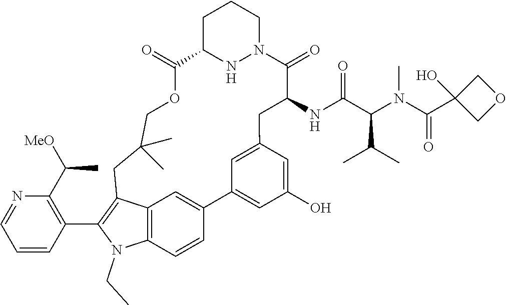 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| B138 | 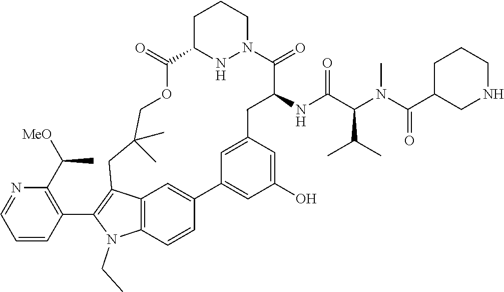 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| B139 | 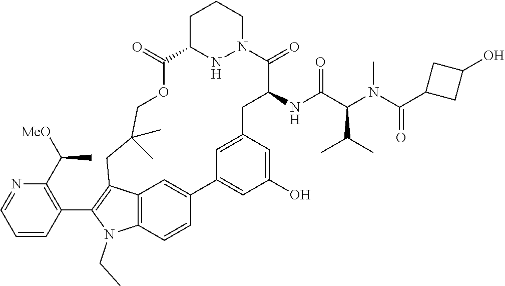 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| B141 | 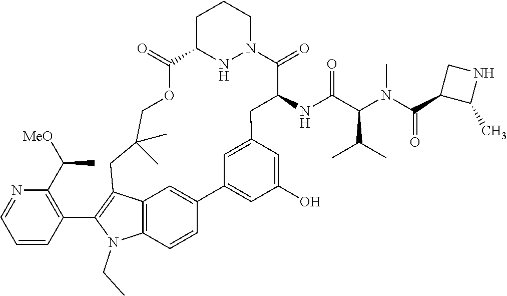 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| B143 | 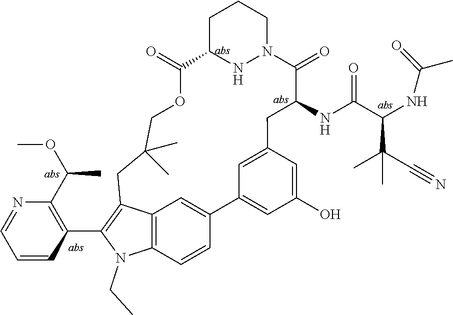 |
| B144 | 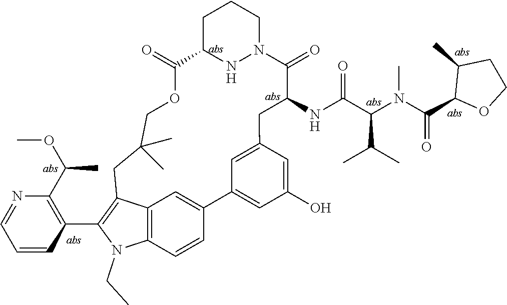 |
| B145 | 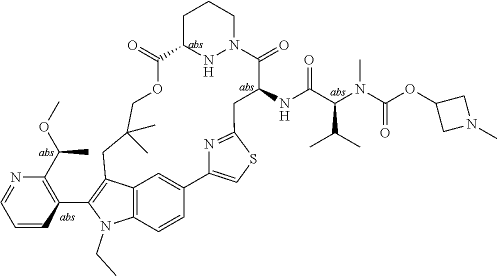 |
| B146 | 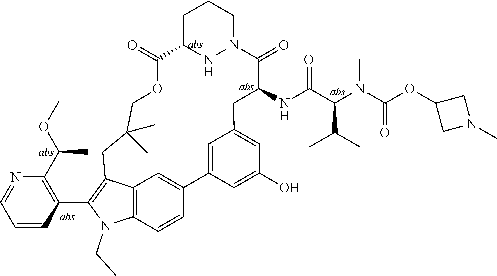 |
| B147 | 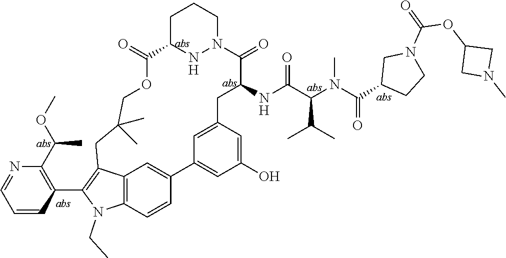 |
| B148 | 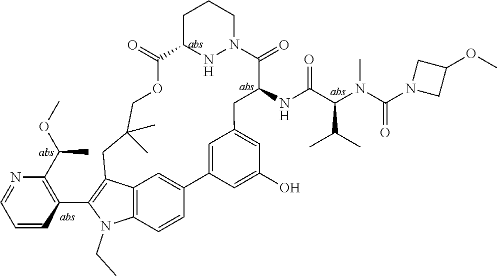 |
| B149 | 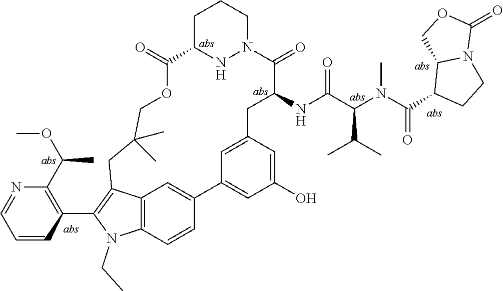 |
| B150 | 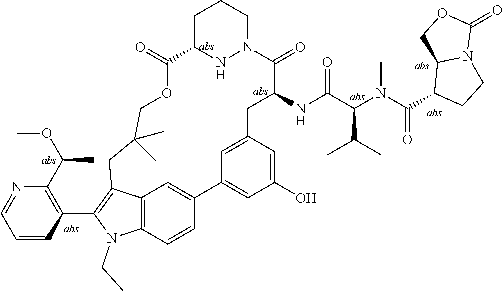 |
| B151 | 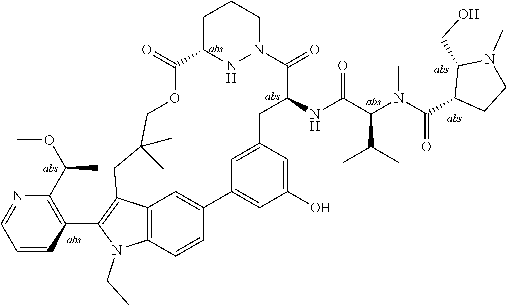 |
| B152 | 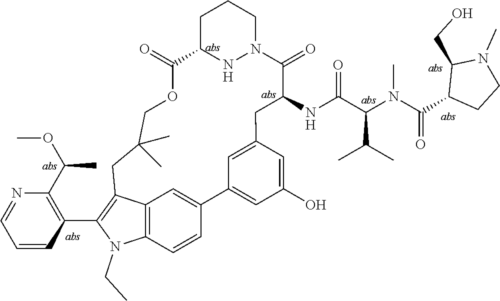 |
| B153 | 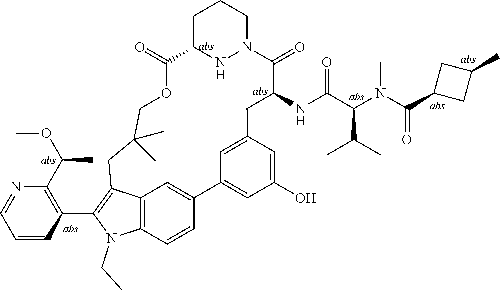 |
| B154 | 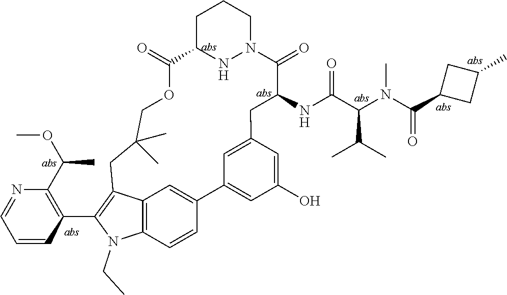 |
| B155 | 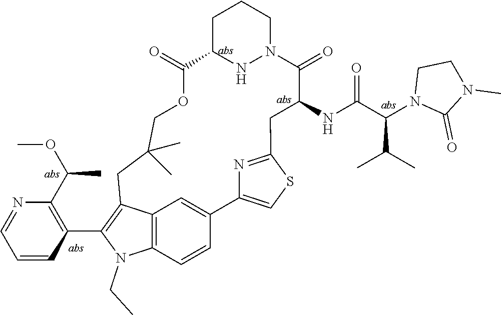 |
| B156 | 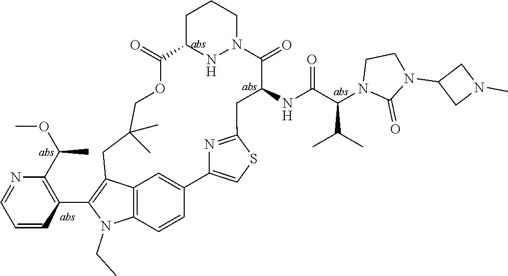 |
| B157 | 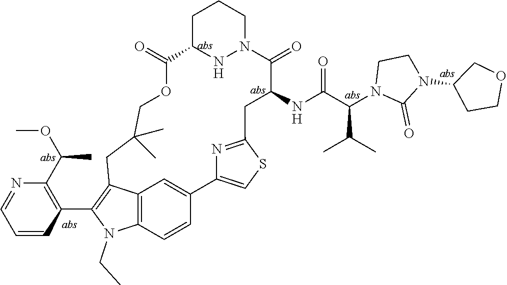 |
| B158 | 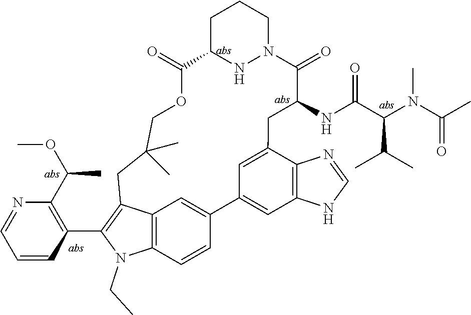 |
| B159 | 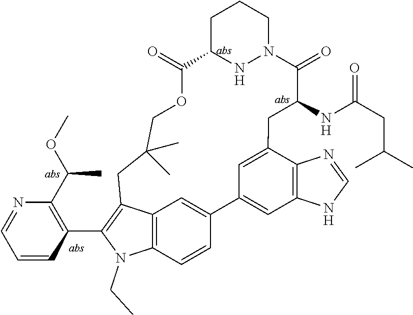 |
| B160 | 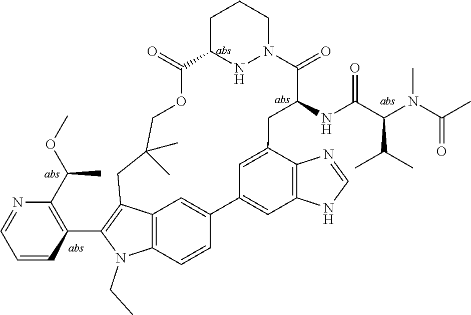 |
| B161 | 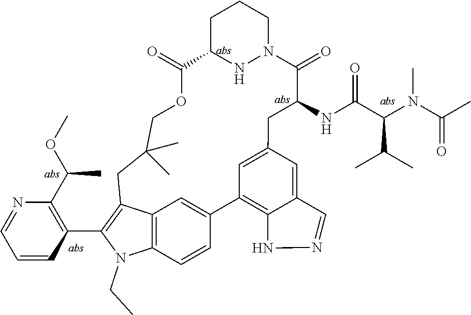 |
| B162 | 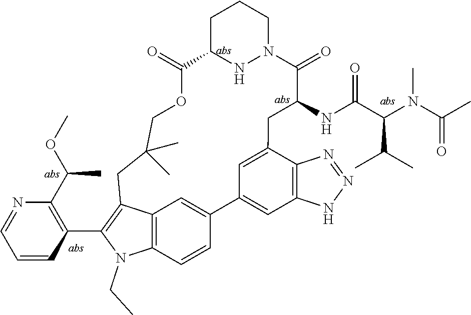 |
| B163 | 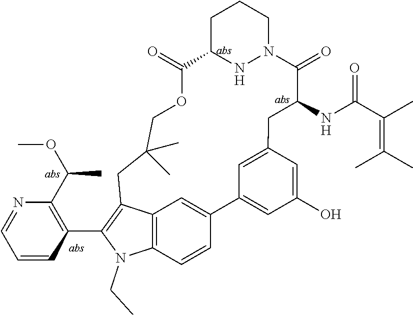 |
| B164 | 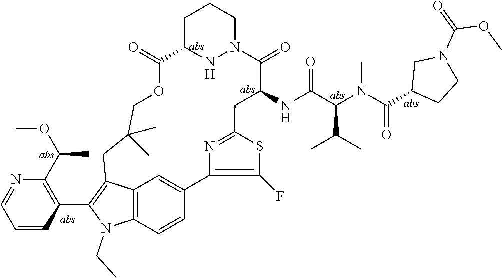 |
| B165 | 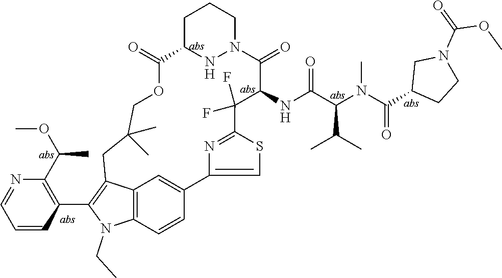 |
| B166 | 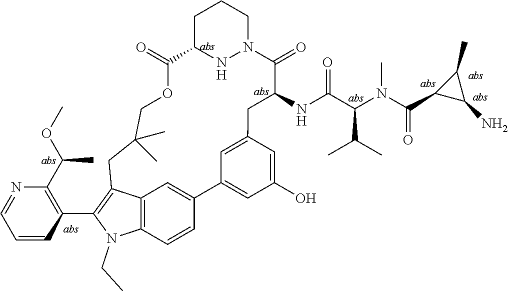 |
| B167 | 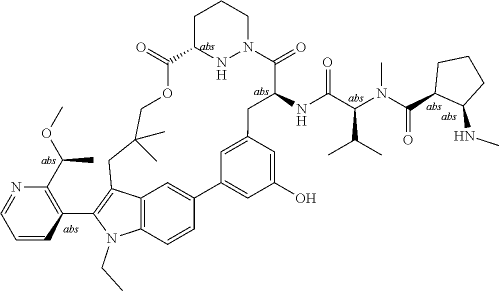 |
| B168 | 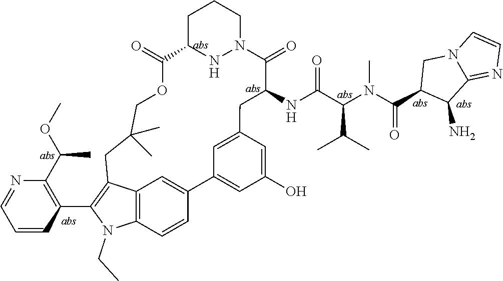 |
| B169 | 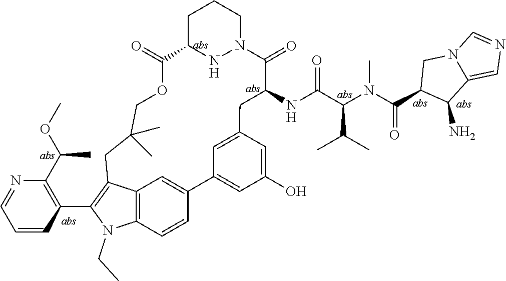 |
| B170 | 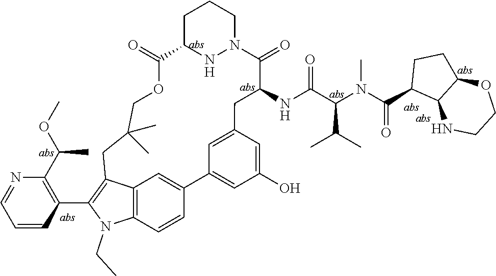 |
| B171 | 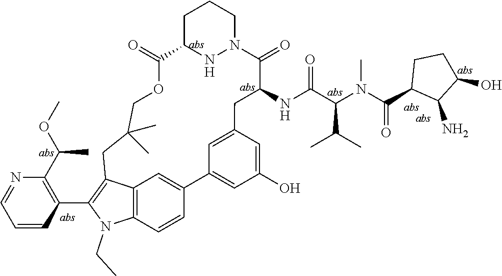 |
| B172 | 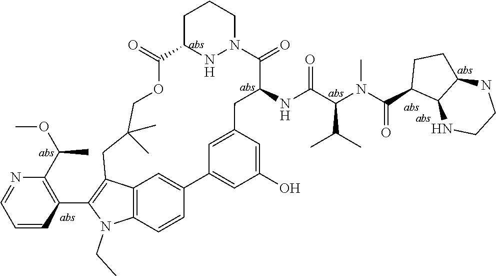 |
| B173 | 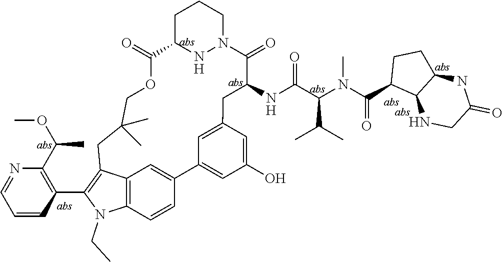 |
| B174 | 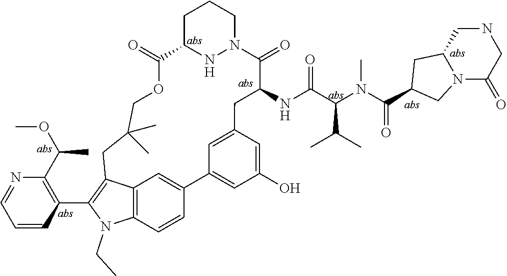 |
| B175 | 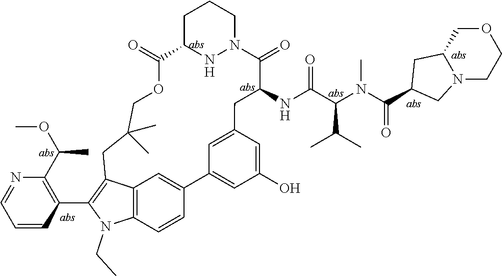 |
| B176 | 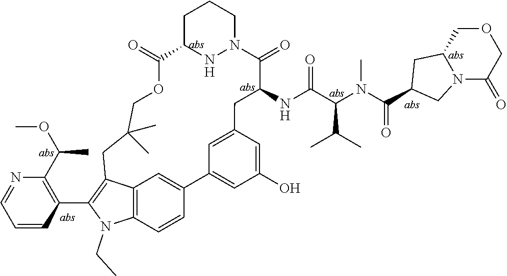 |
| B177 | 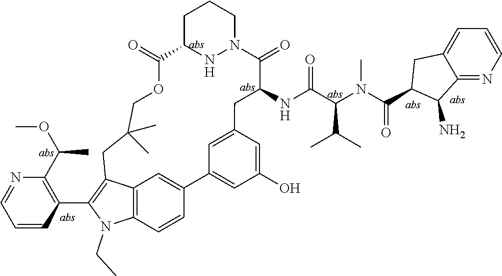 |
| B178 | 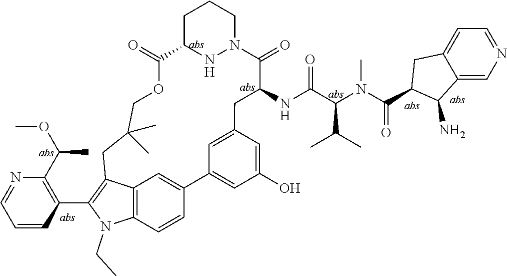 |
| B179 | 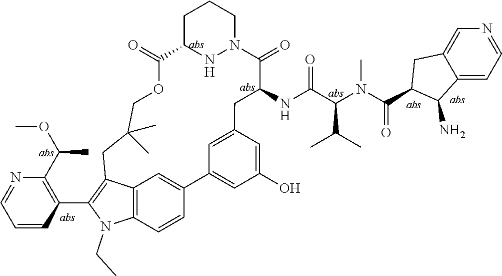 |
| B180 | 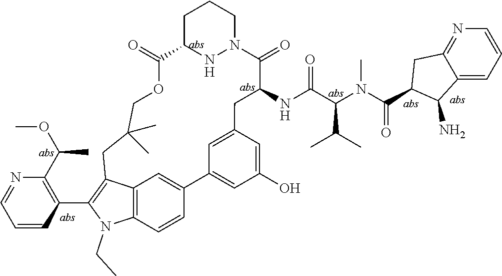 |
| B181 | 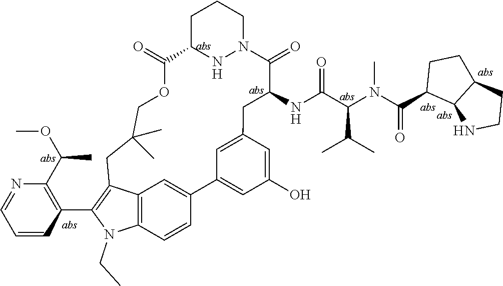 |
| B182 | 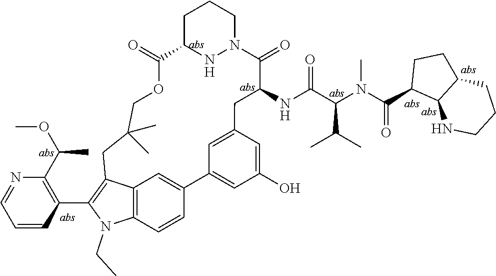 |
| B183 | 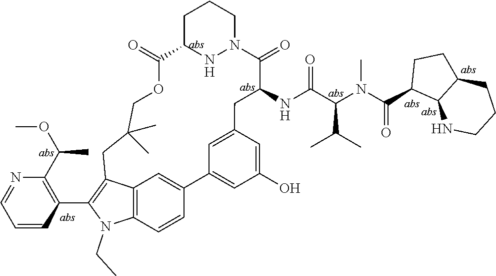 |
| B184 | 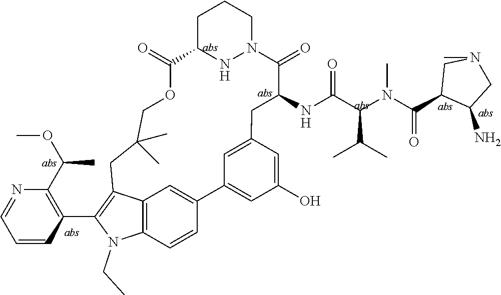 |
| B185 | 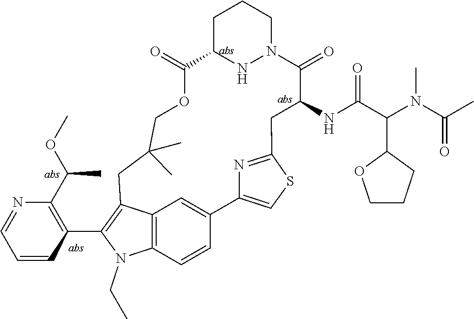 |
| B186 | 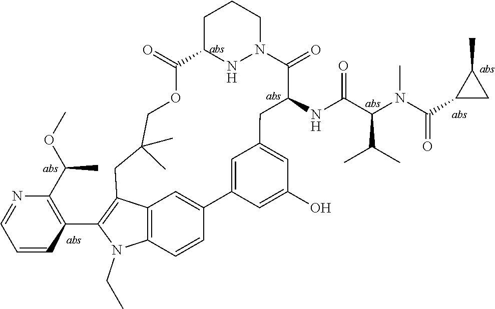 |
| B187 | 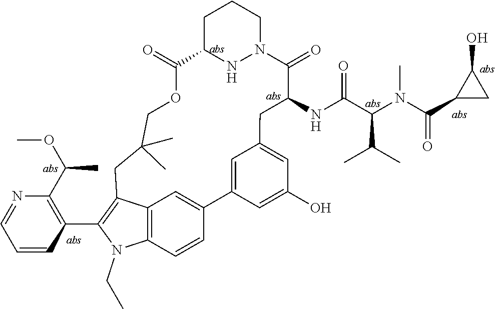 |
| B188 | 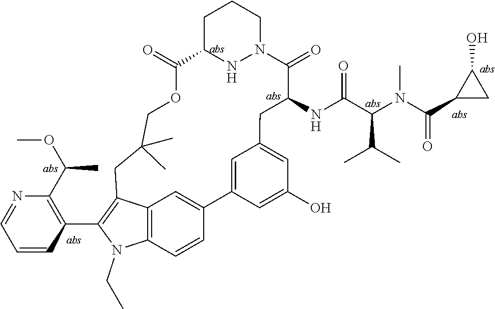 |
| B189 | 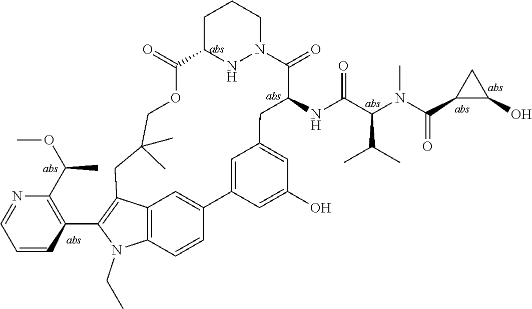 |
| B190 | 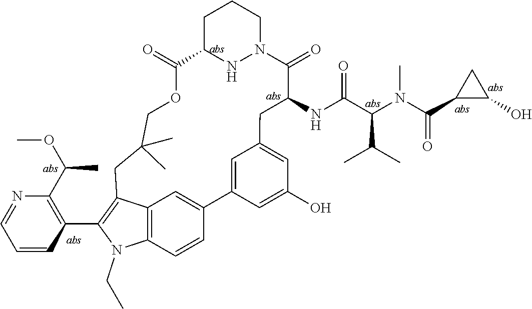 |
| B191 | 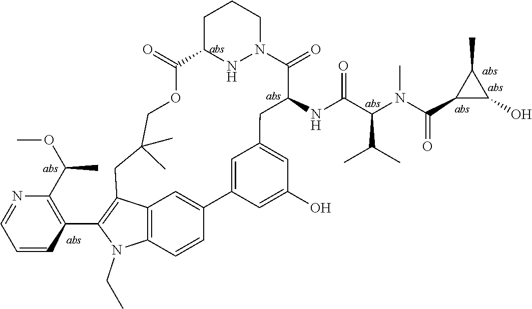 |
| B192 | 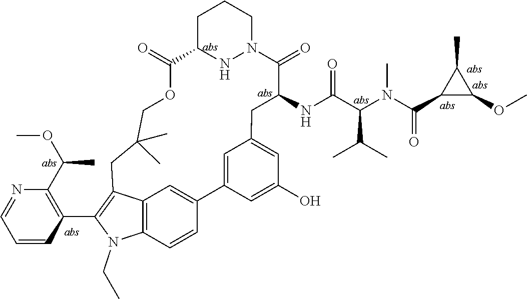 |
| B193 | 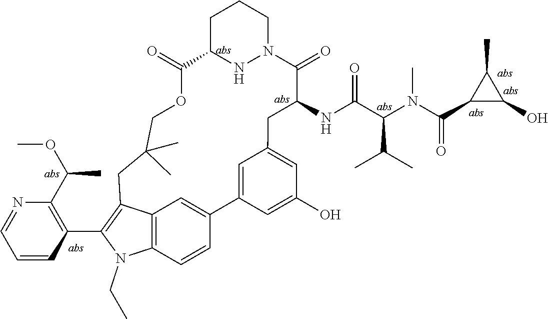 |
| B194 | 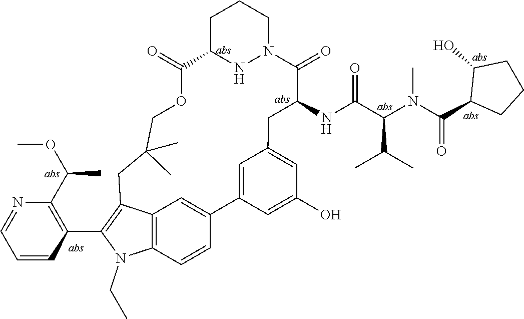 |
| B195 | 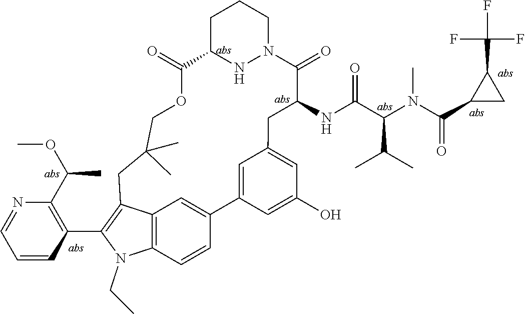 |
| B196 | 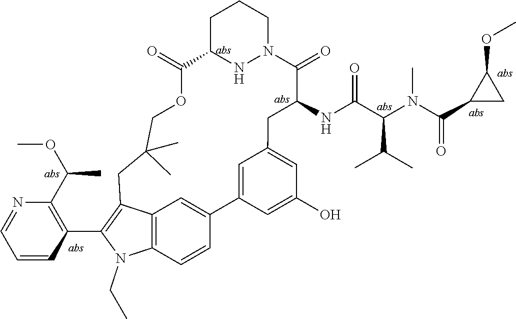 |
| B197 | 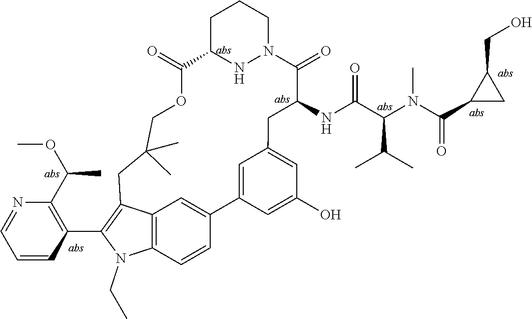 |
| B198 | 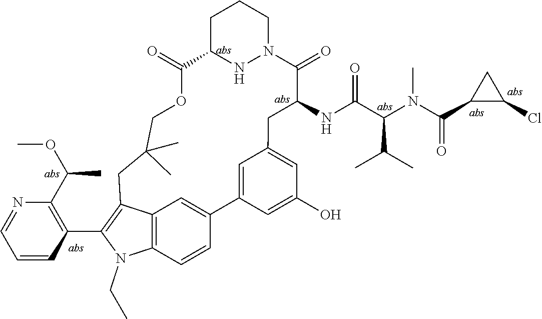 |
| B199 | 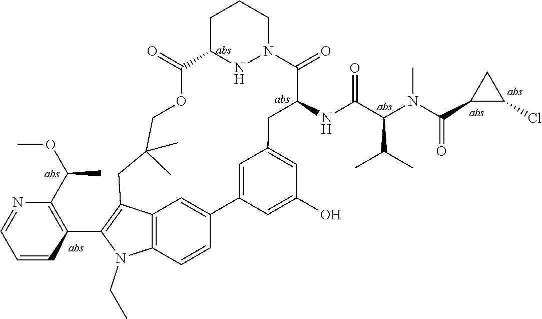 |
| B200 | 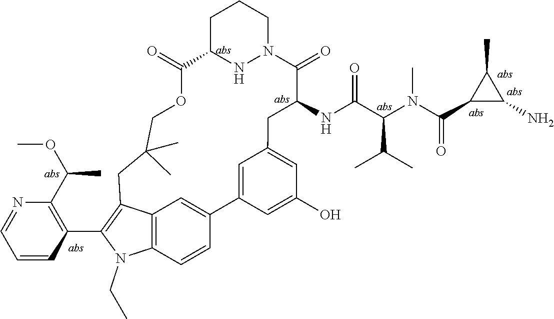 |
| B201 | 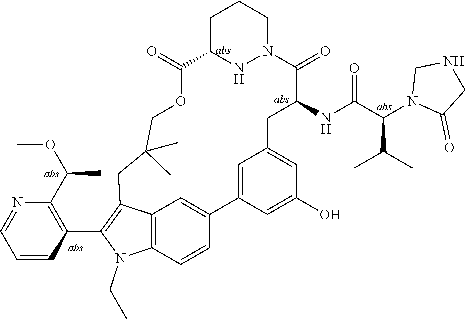 |
| B202 | 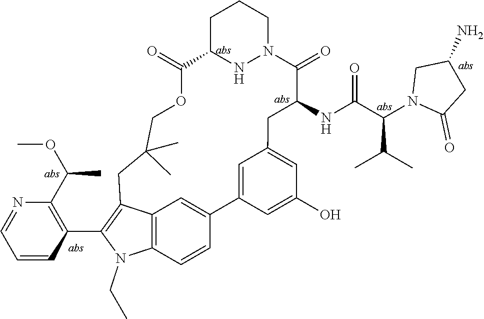 |
| B203 | 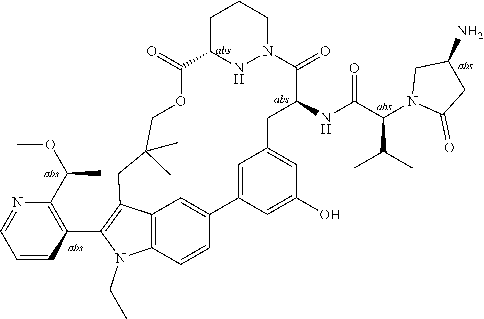 |
| B204 | 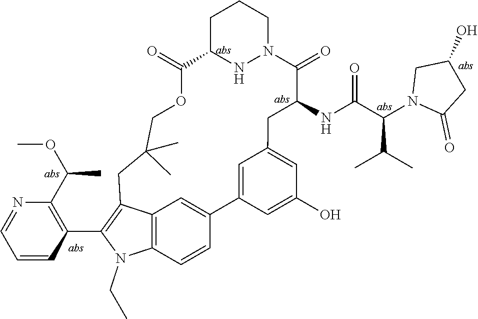 |
| B205 | 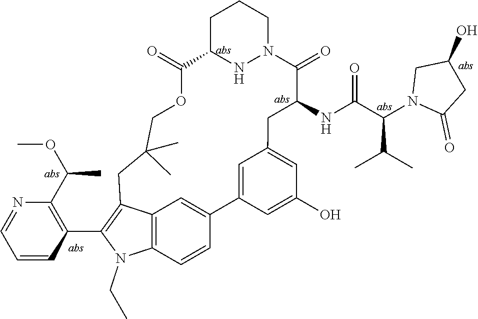 |
| B206 | 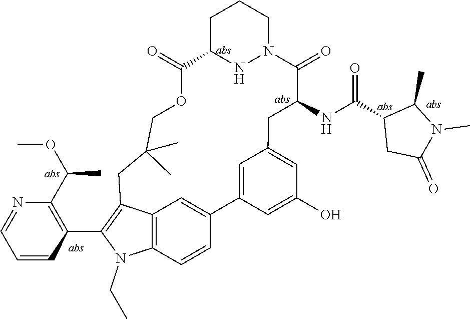 |
| B207 | 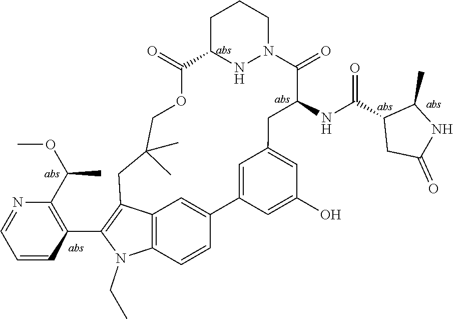 |
| B208 | 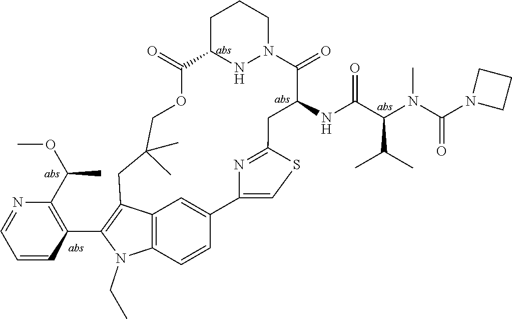 |
| B209 | 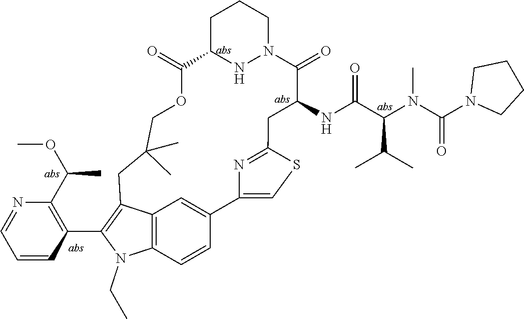 |
| B210 | 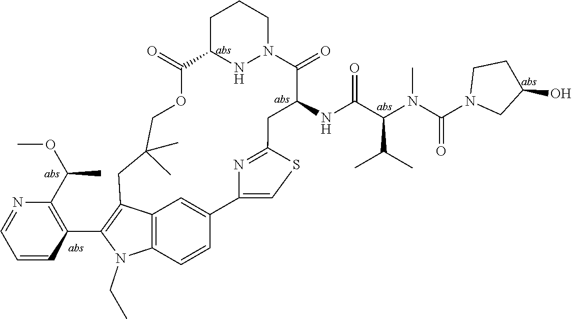 |
| B211 | 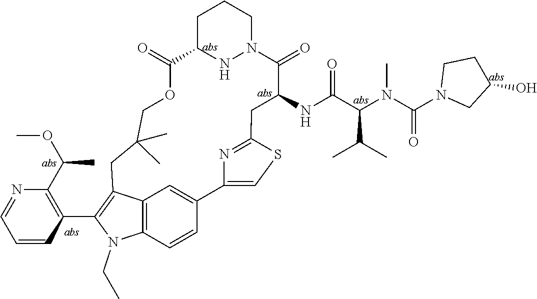 |
| B212 | 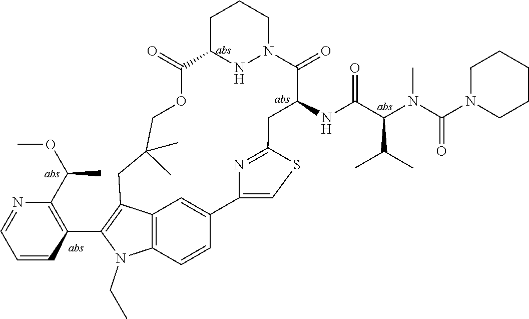 |
| B213 | 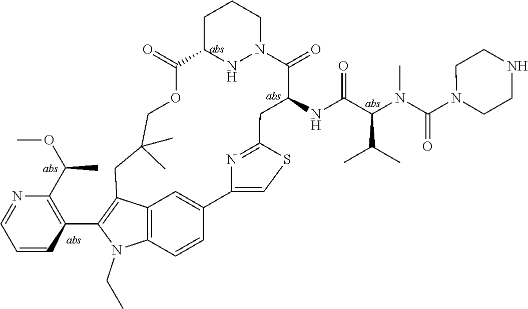 |
| B214 | 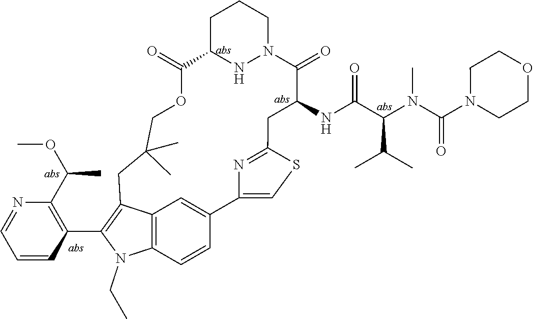 |
| B215 | 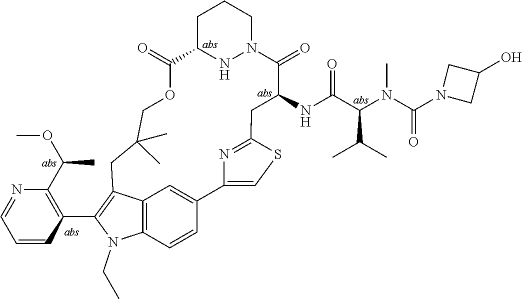 |
| B216 | 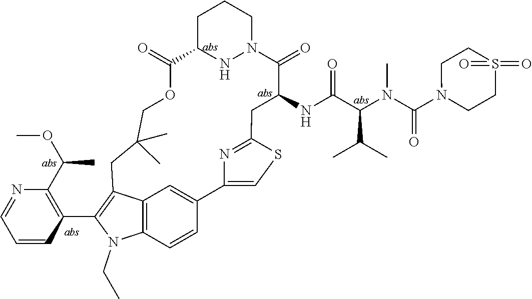 |
| B217 | 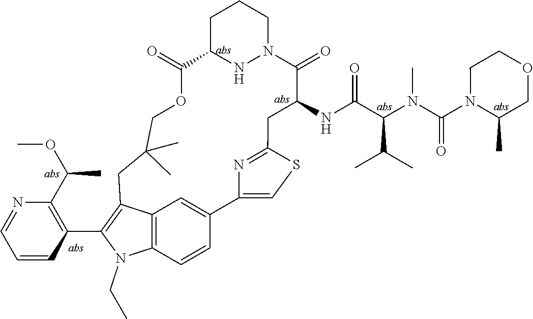 |
| B218 | 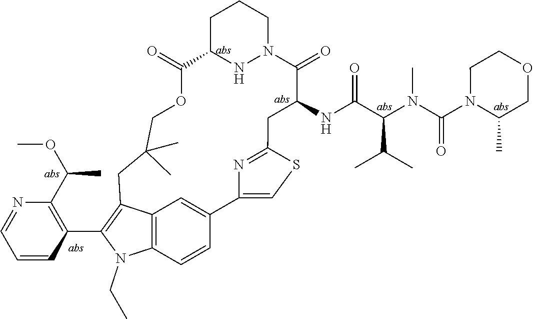 |
| B219 | 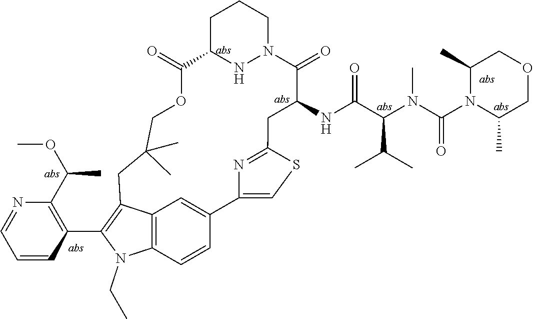 |
| B220 | 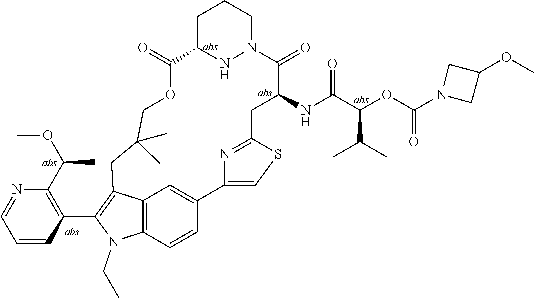 |
| B221 | 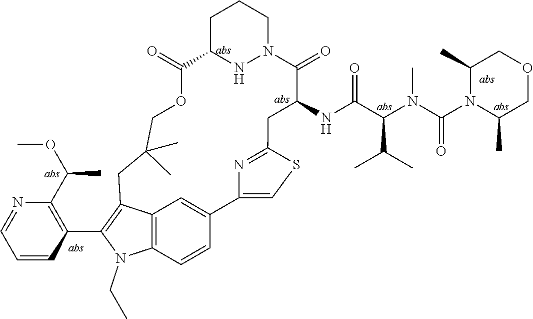 |
| B222 | 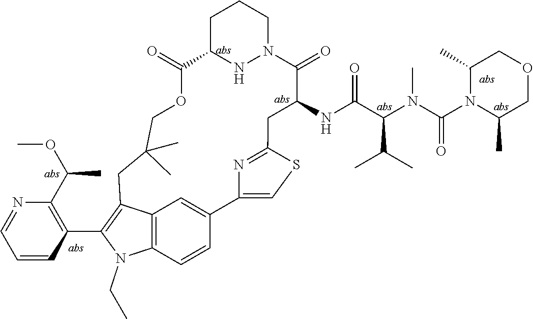 |
| B223 | 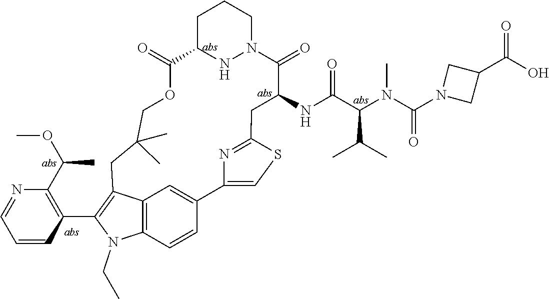 |
| B224 | 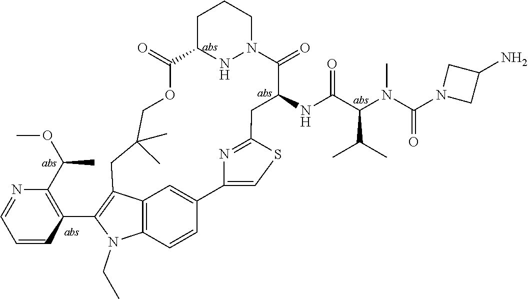 |
| B225 | 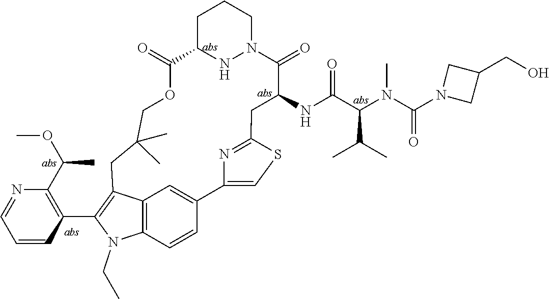 |
| B226 | 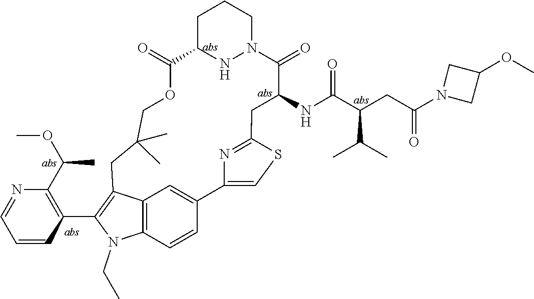 |
| B227 | 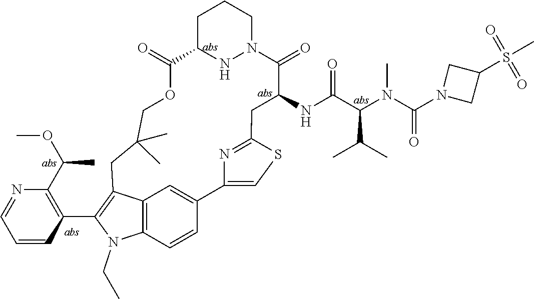 |
| B228 | 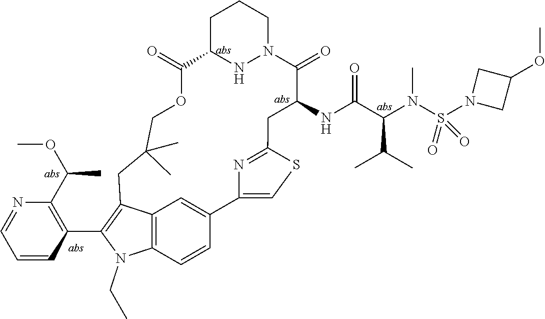 |
| B229 | 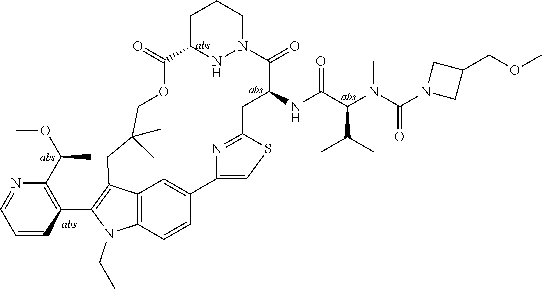 |
| B230 | 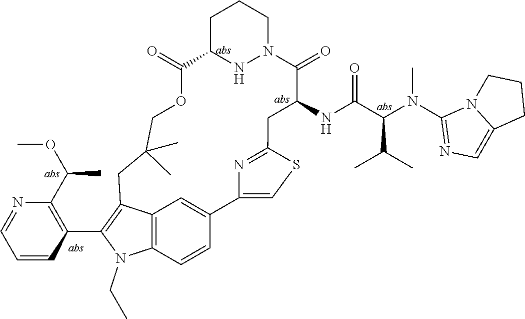 |
| B231 | 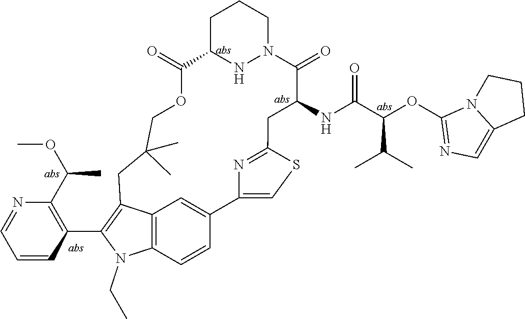 |
| B232 | 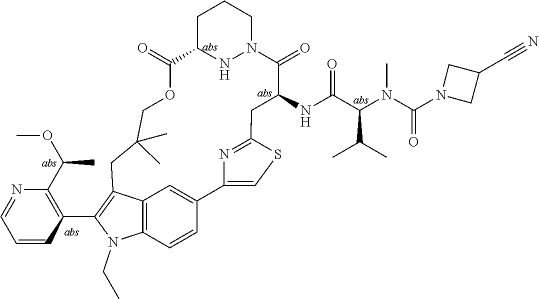 |
| B233 | 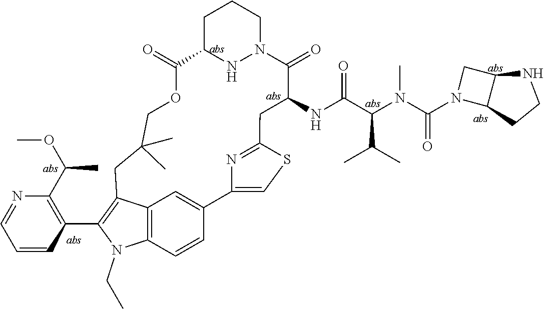 |
| B234 | 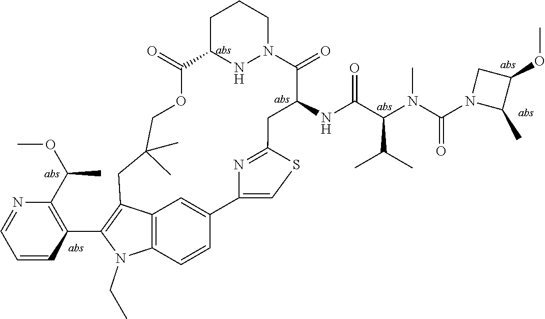 |
| B235 | 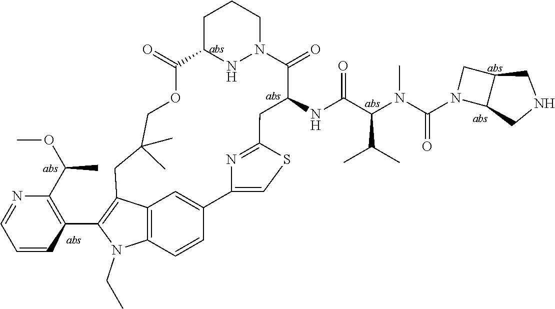 |
| B236 | 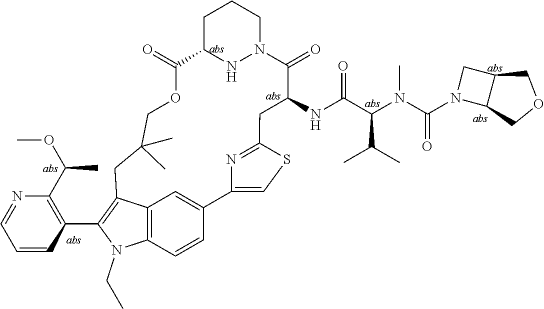 |
| B237 | 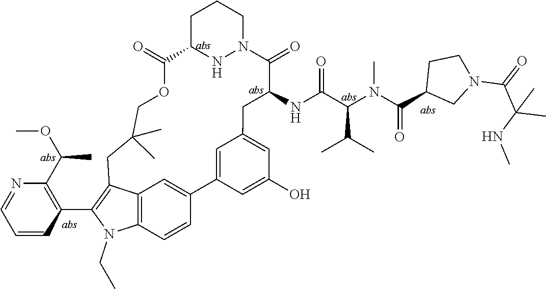 |
| B238 | 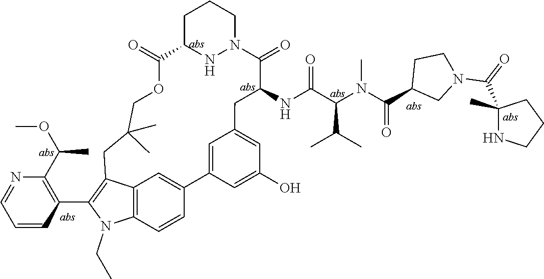 |
| B239 | 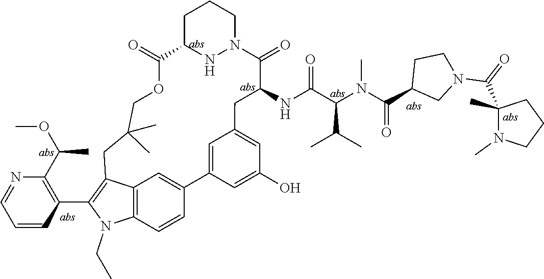 |
| B240 | 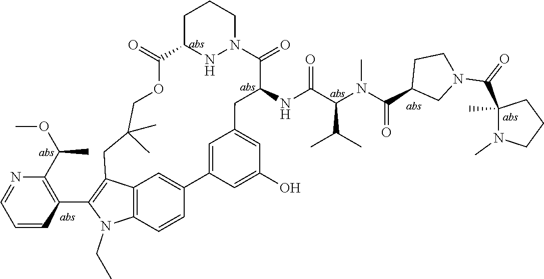 |
| B241 | 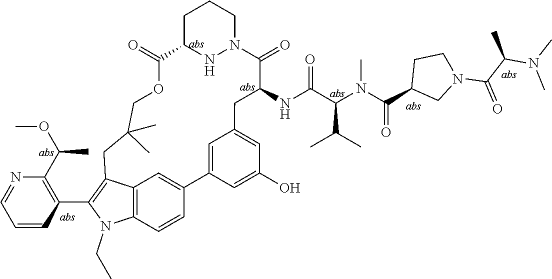 |
| B242 | 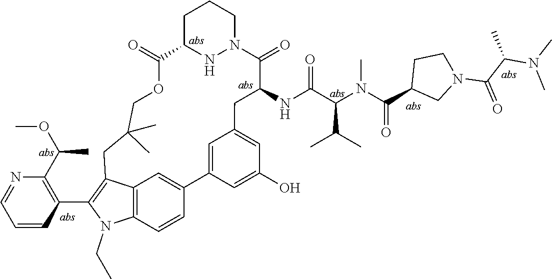 |
| B243 | 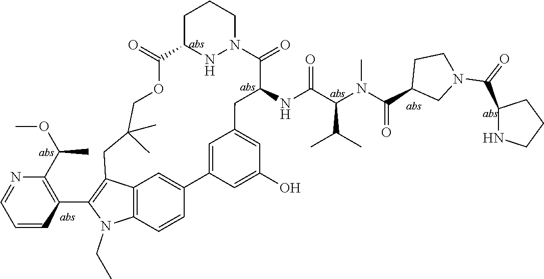 |
| B244 | 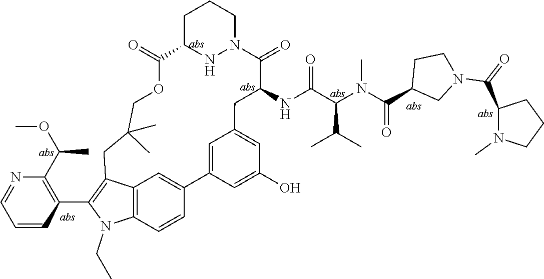 |
| B245 | 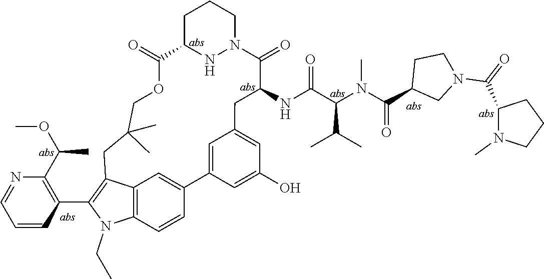 |
| B246 | 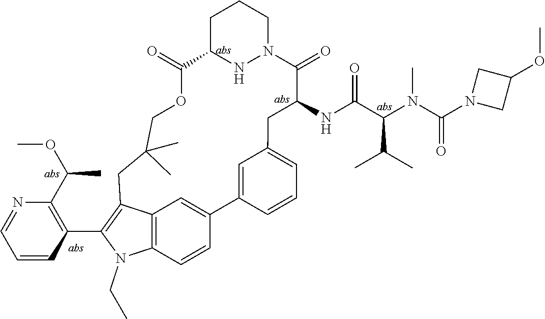 |
| B247 | 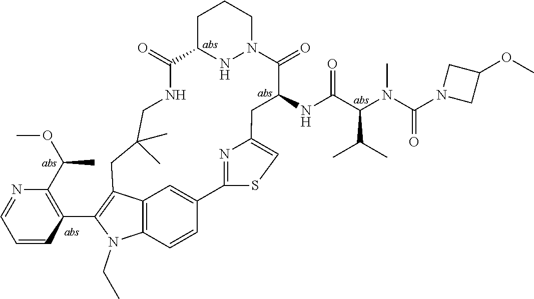 |
| B248 | 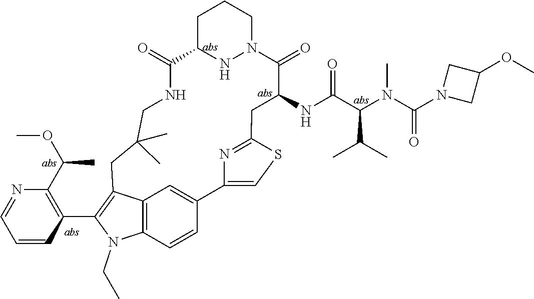 |
| B249 | 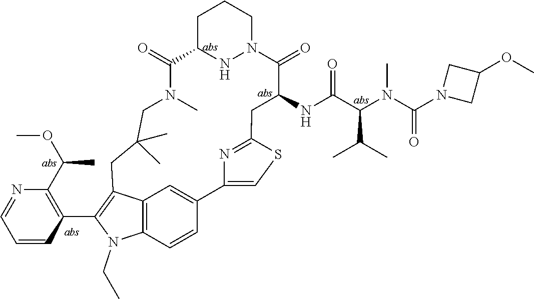 |
| B250 | 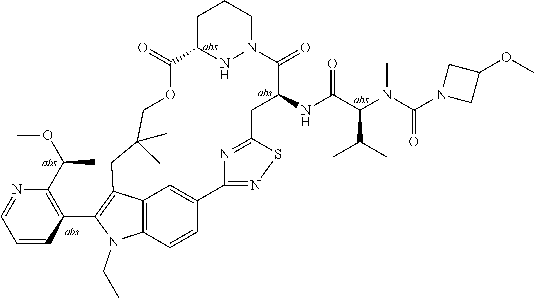 |
| B251 | 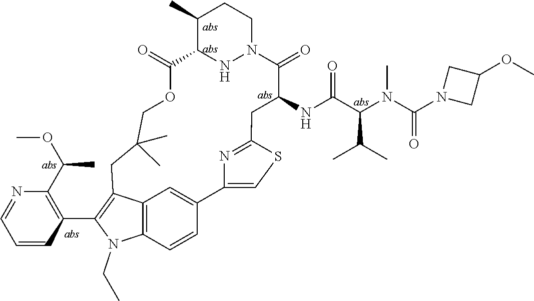 |
| B252 | 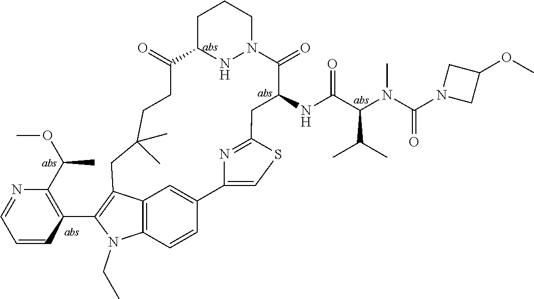 |
| B253 | 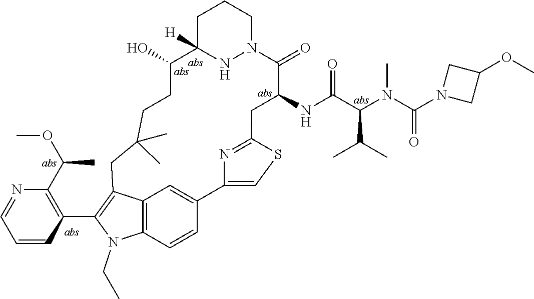 |
| B254 | 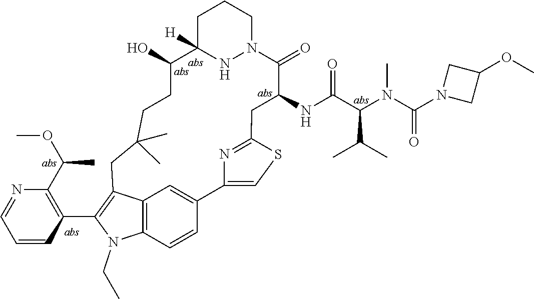 |
| B255 | 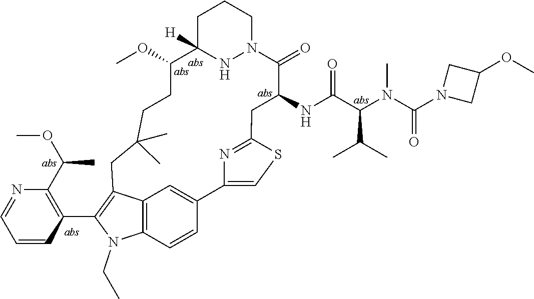 |
| B256 | 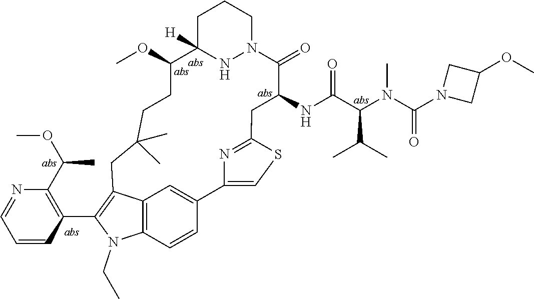 |
| B257 | 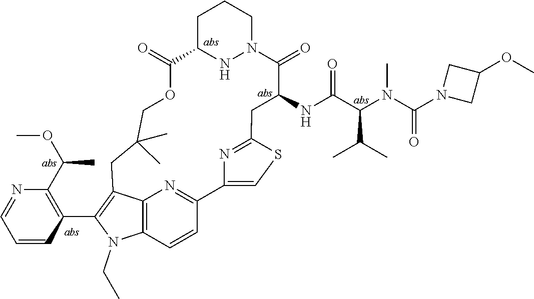 |
| B258 | 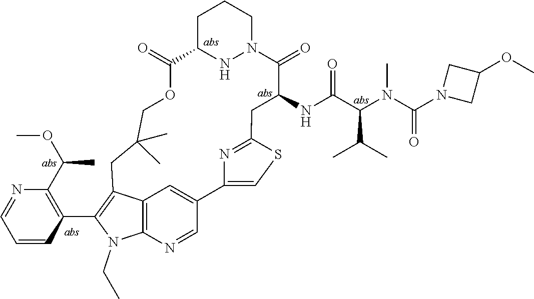 |
| B259 | 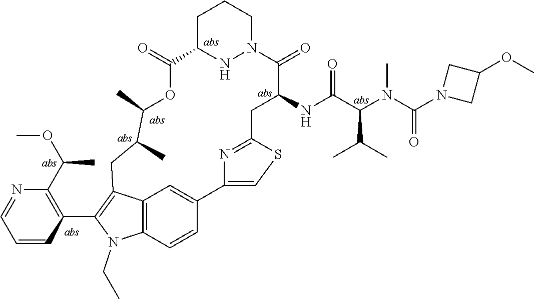 |
| B260 | 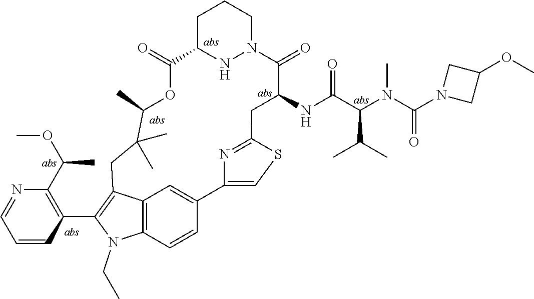 |
| B261 | 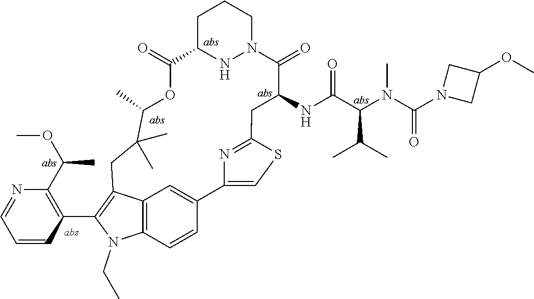 |
| B262 | 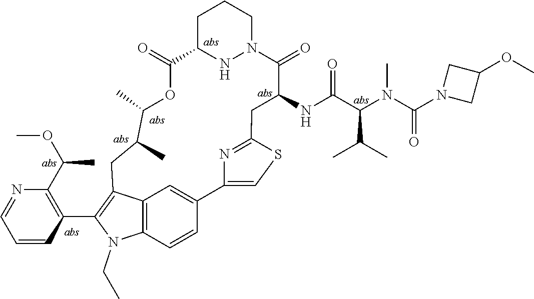 |
| B263 | 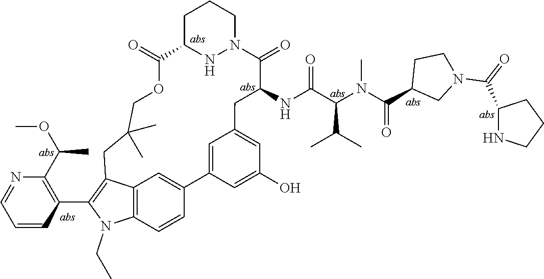 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Table 1 herein were prepared using methods disclosed herein or were prepared using methods disclosed herein combined with the knowledge of one of skill in the art.

Compounds of Table 2 may be prepared using methods disclosed herein or may be prepared using methods disclosed herein combined with the knowledge of one of skill in the art.

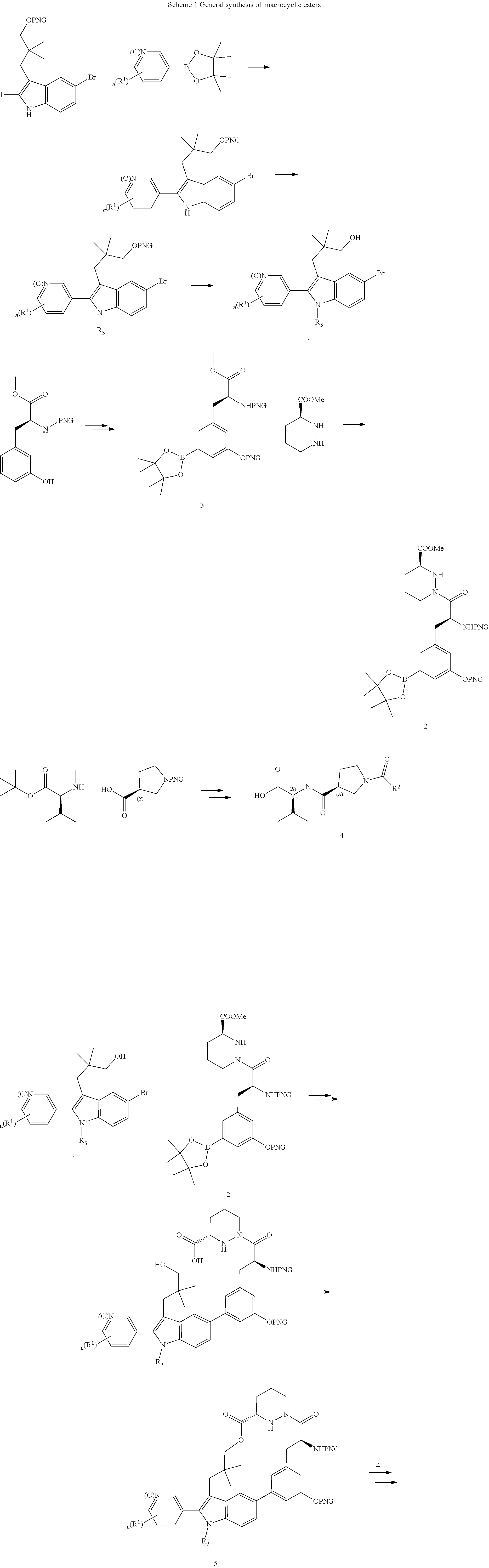

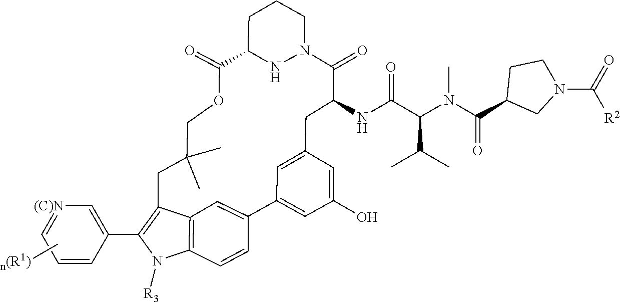

A general synthesis of macrocyclic esters is outlined in Scheme 1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions. Methyl-amino-hexahydropyridazine-3-carboxylate-boronic ester (2) can be prepared in three steps, including protection, Iridium catalyst mediated borylation, and coupling with methyl(S)-hexahydropyridazine-3-carboxylate.

An appropriately substituted acetylpyrrolidine-3-carbonyl-N-methyl-L-valine (4) can be made by coupling of methyl-L-valinate and protected(S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired-B-L-W group of a compound of Formula (I), where B, L and W are defined herein, including by using methods exemplified in the Example section herein.

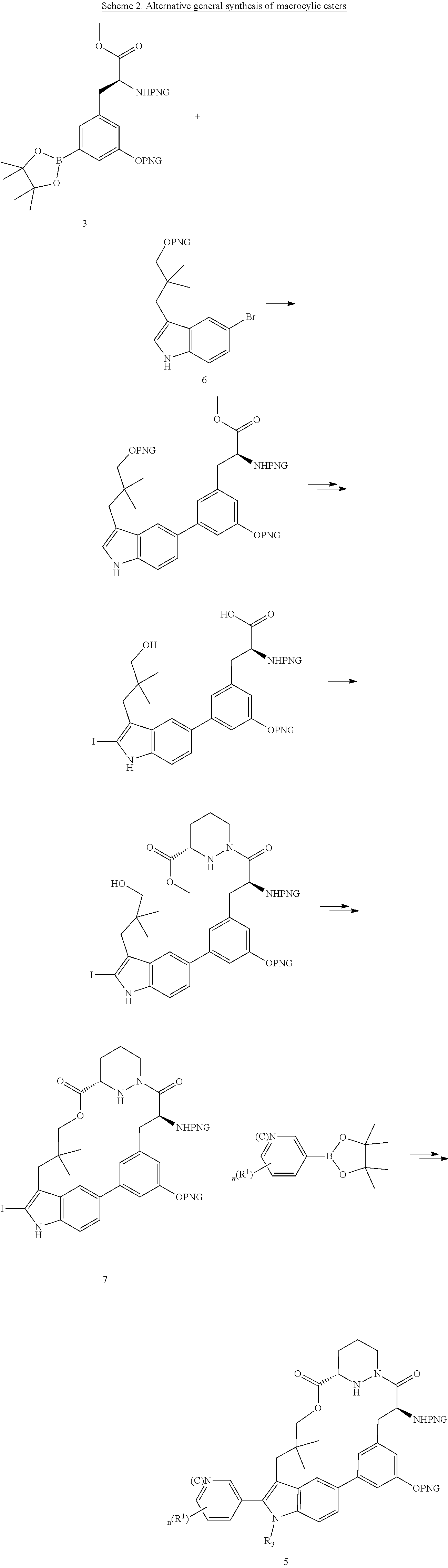

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl(S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired-B-L-W group of a compound of Formula (I), where B, L and W are defined herein, including by using methods exemplified in the Example section herein.

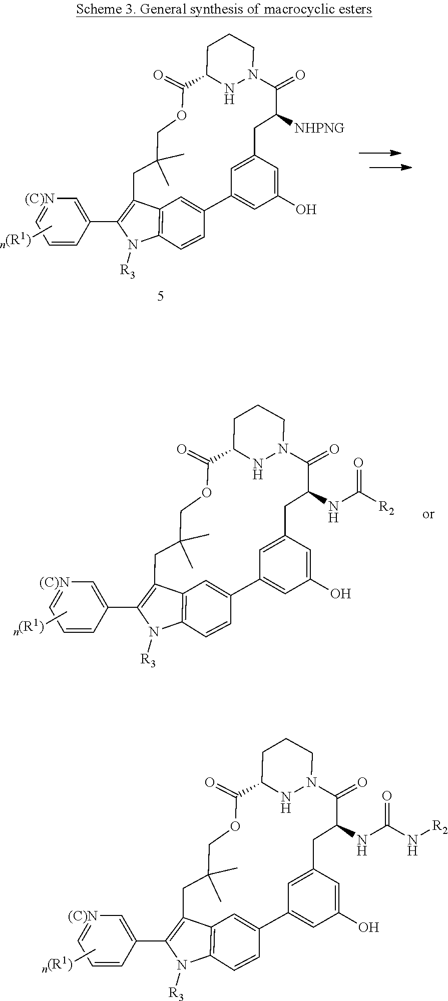

Alternatively, fully a protected macrocycle (5) can be deprotected and coupled with an appropriately substitututed coupling partners, and deprotected to results in a macrocyclic product. Additional deprotection or functionalization steps are be required to produce a final compound. For example, a person of skill in the art would be able to install into a macrocyclic ester a desired-B-L-W group of a compound of Formula (I), where B, L and W are defined herein, including by using methods exemplified in the Example section herein.

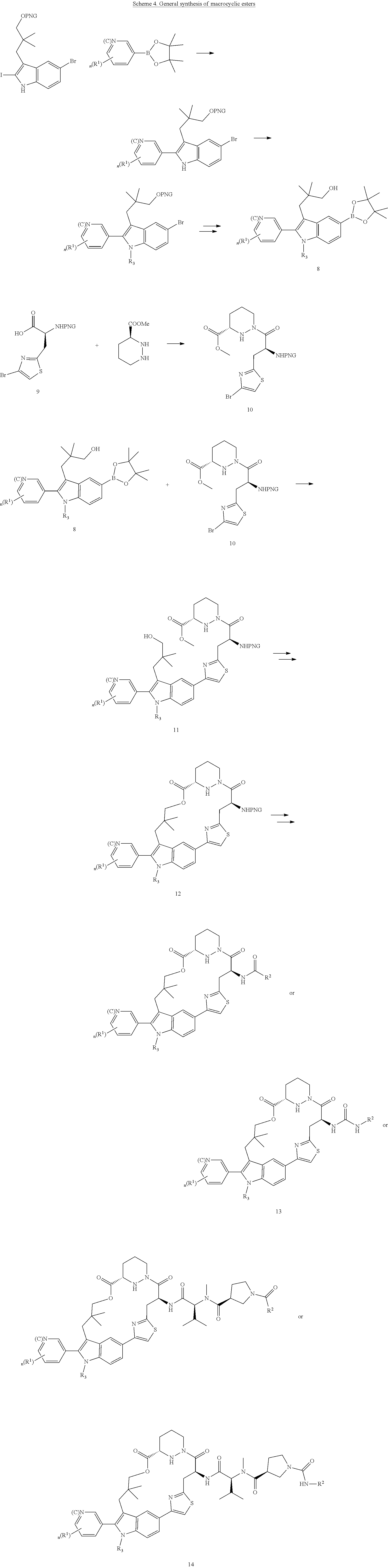

An alternative general synthesis of macrocyclic esters is outlined in Scheme 4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl) propanoyl) hexahydropyridazine-3-carboxylate (10) can be prepared via coupling of(S)-2-amino-3-(4-bromothiazol-2-yl) propanoic acid (9) with methyl (S)-hexahydropyridazine-3-carboxylate.

The final macrocyclic esters can be made by coupling of Methyl-amino-3-(4-bromothiazol-2-yl) propanoyl) hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

### Numbered Embodiments

[1] A compound, or pharmaceutically acceptable salt thereof, having the structure of Formula I:

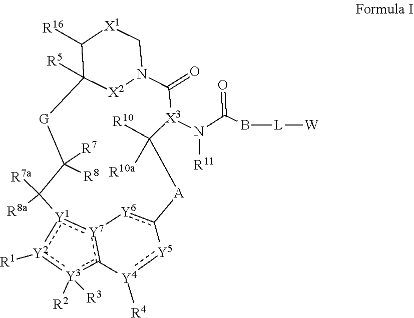

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R¹⁶ is hydrogen or C₁-C₃ alkyl.
[2] The compound, or pharmaceutically acceptable salt thereof, of paragraph [1], wherein G is optionally substituted C₁-C₄ heteroalkylene.
[3] The compound, or pharmaceutically acceptable salt thereof, of paragraph [1] or [2], wherein the compound has the structure of Formula Ic:

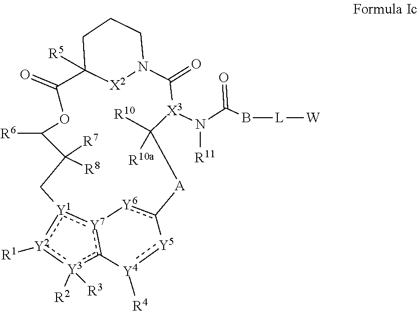

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.
[4] The compound, or pharmaceutically acceptable salt thereof, of any one of paragraphs [1] to [3], wherein X² is NH.
[5] The compound, or pharmaceutically acceptable salt thereof, of any one of paragraphs [1] to [4], wherein X³ is CH.
[6] The compound, or pharmaceutically acceptable salt thereof, of any one of paragraphs [1] to [5], wherein R¹¹ is hydrogen.
[7] The compound, or pharmaceutically acceptable salt thereof, of any one of paragraphs [1] to [5], wherein R¹¹ is C₁-C₃ alkyl.
[8] The compound, or pharmaceutically acceptable salt thereof, of paragraph [7], wherein R¹¹ is methyl.
[9] The compound, or pharmaceutically acceptable salt thereof, of any one of paragraphs [1] to [6], wherein the compound has the structure of Formula Id:

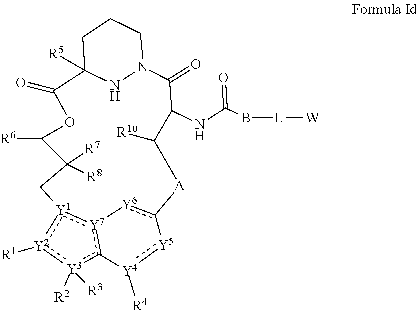

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.
[10] The compound, or pharmaceutically acceptable salt thereof, of any one of paragraphs [1] to [9] wherein X¹ is optionally substituted C₁-C₂ alkylene.
[11] The compound, or pharmaceutically acceptable salt thereof, of paragraph [10], wherein X¹ is methylene.
[12] The compound, or pharmaceutically acceptable salt thereof, of any one of paragraphs [1] to [11], wherein R⁵ is hydrogen.
[13] The compound, or pharmaceutically acceptable salt thereof, of any one of paragraphs [1] to [11], wherein R⁵ is C₁-C₄ alkyl optionally substituted with halogen.
[14] The compound, or pharmaceutically acceptable salt thereof, of paragraph [13], wherein R⁵ is methyl.
[15] The compound, or pharmaceutically acceptable salt thereof, of any one of paragraphs [1] to [14], wherein Y⁴ is C.
[16] The compound, or pharmaceutically acceptable salt thereof, of any one of paragraphs [1] to [15], wherein R⁴ is hydrogen.
[17] The compound, or pharmaceutically acceptable salt thereof, of any one of paragraphs [1] to [16], wherein Y⁵ is CH.
[18] The compound, or pharmaceutically acceptable salt thereof, of any one of paragraphs [1] to [17], wherein Y⁶ is CH.
[19] The compound, or pharmaceutically acceptable salt thereof, of any one of paragraphs [1] to [18], wherein Y¹ is C.
[20] The compound, or pharmaceutically acceptable salt thereof, of any one of paragraphs [1] to [19], wherein Y² is C.
[21] The compound, or pharmaceutically acceptable salt thereof, of any one of paragraphs [1] to [20], wherein Y³ is N.
[22] The compound, or pharmaceutically acceptable salt thereof, of any one of paragraphs [1] to [21], wherein R³ is absent.
[23] The compound, or pharmaceutically acceptable salt thereof, of any one of paragraphs [1] to [22], wherein Y⁷ is C.
[24] The compound, or pharmaceutically acceptable salt thereof, of any one of paragraphs [1] to [6] or [9] to [23], wherein the compound has the structure of Formula I:

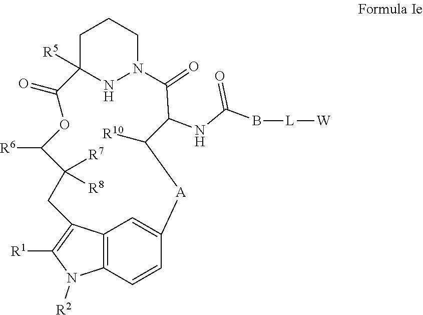

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.
[25] The compound, or pharmaceutically acceptable salt thereof, of any one of paragraphs [3] to [24], wherein R⁶ is hydrogen.
[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.
[27] The compound, or pharmaceutically acceptable salt thereof, of paragraph [26], wherein R² is optionally substituted C₁-C₆ alkyl.
[28] The compound, or pharmaceutically acceptable salt thereof, of paragraph [27], wherein R² is ethyl.
[29] The compound, or pharmaceutically acceptable salt thereof, of any one of paragraphs [1] to [28], wherein R⁷ is optionally substituted C₁-C₃ alkyl.
[30] The compound, or pharmaceutically acceptable salt thereof, of paragraph [29], wherein R⁷ is C₁-C₃ alkyl.
[31] The compound, or pharmaceutically acceptable salt thereof, of any one of paragraphs [1] to [30], wherein R⁸ is optionally substituted C₁-C₃ alkyl.
[32] The compound, or pharmaceutically acceptable salt thereof, of paragraph [31], wherein R⁸ is C₁-C₃ alkyl.
[33] The compound, or pharmaceutically acceptable salt thereof, of any one of paragraphs [1] to [32], wherein the compound has the structure of Formula If:

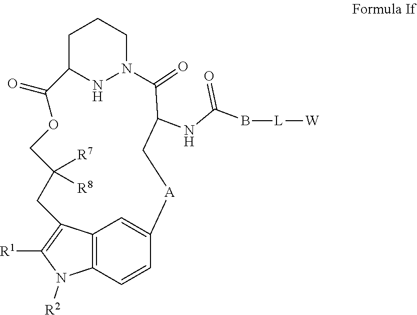

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[34] The compound, or pharmaceutically acceptable salt thereof, of any one of paragraphs [1] to [33], wherein R¹ is 5 to 10-membered heteroaryl.
[35] The compound, or pharmaceutically acceptable salt thereof, of paragraph [34], wherein R¹ is optionally substituted 6-membered aryl or optionally substituted 6-membered heteroaryl.
[36] The compound, or pharmaceutically acceptable salt thereof, of any one of paragraphs [1] to [35], wherein the compound has the structure of Formula Ig:

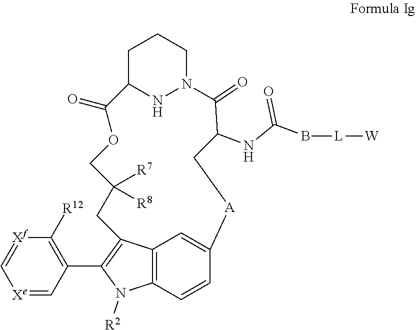

wherein A is, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.
[37] The compound, or pharmaceutically acceptable salt thereof, of paragraph [36], wherein X^{e} is N and X^{f} is CH.
[38] The compound, or pharmaceutically acceptable salt thereof, of paragraph [36], wherein X^{e} is CH and X^{f} is N.
[39] The compound, or pharmaceutically acceptable salt thereof, of paragraph [36], wherein X^{e} is CR¹⁷ and X^{f} is N.
[40] The compound, or pharmaceutically acceptable salt thereof, of any one of paragraphs [36] to [39], wherein R¹² is optionally substituted C₁-C₆ heteroalkyl.
[41] The compound, or pharmaceutically acceptable salt thereof, of any one of paragraphs [36] to [40], wherein R¹² is

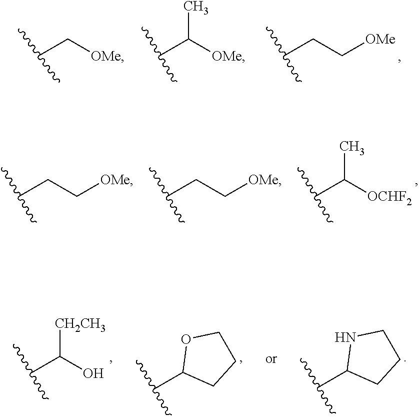

[42] The compound, or pharmaceutically acceptable salt thereof, of any one of paragraphs [1] to [41], wherein the compound has the structure of Formula Ih:

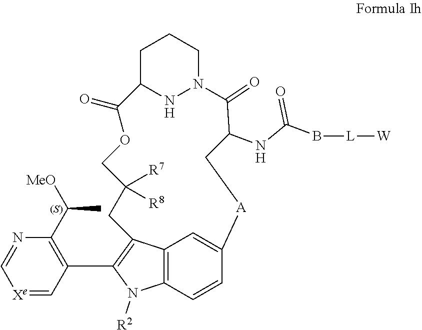

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₈alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.
[43] The compound, or pharmaceutically acceptable salt thereof, of any one of paragraphs [1] to [42], wherein the compound has the structure of Formula Ii:

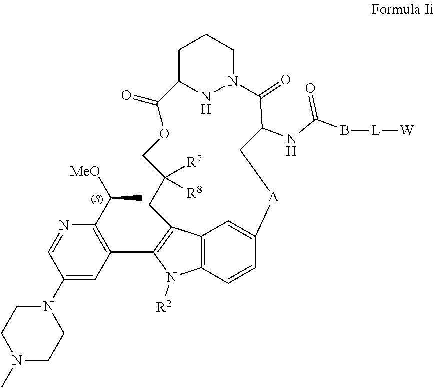

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[44] The compound, or pharmaceutically acceptable salt thereof, of any one of paragraphs [1] to [43], wherein A is optionally substituted 6-membered arylene.
[45] The compound, or pharmaceutically acceptable salt thereof, of paragraph [44], wherein A has the structure:

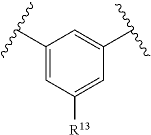

wherein R¹³ is hydrogen, hydroxy, amino, cyano, optionally substituted C₁-C₆ alkyl, or optionally substituted C₁-C₆ heteroalkyl.
[46] The compound, or pharmaceutically acceptable salt thereof, of paragraph [45], wherein R¹³ is hydrogen.
[47] The compound, or pharmaceutically acceptable salt thereof, of paragraph [45], wherein R¹³ is hydroxy.
[48] The compound, or pharmaceutically acceptable salt thereof, of any one of paragraphs [1] to [43], wherein A is optionally substituted 5 to 6-membered heteroarylene.
[49] The compound, or pharmaceutically acceptable salt thereof, of paragraph [48], wherein A is:

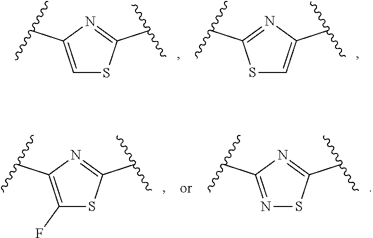 [SMILES: *c1csc(*)n1 ; *c1csc(*)n1 ; *c1nc(*)c(F)s1 ; *c1nsc(*)n1]

[50] The compound, or pharmaceutically acceptable salt thereof, of paragraph [49], wherein A is

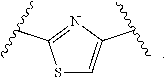 [SMILES: *c1csc(*)n1]

[51] The compound, or pharmaceutically acceptable salt thereof, of any one of paragraphs [1] to [50], wherein B is —CHR⁹—.
[52] The compound, or pharmaceutically acceptable salt thereof, of paragraph [51], wherein R⁹ is optionally substituted C₁-C₆ alkyl or optionally substituted 3 to 6-membered cycloalkyl.
[53] The compound, or pharmaceutically acceptable salt thereof, of paragraph [52], wherein R⁹ is:

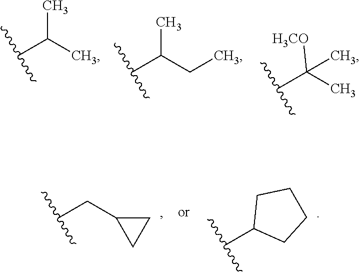

[54] The compound, or pharmaceutically acceptable salt thereof, of paragraph [53], wherein R⁹ is:

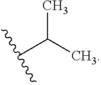 [SMILES: *C(C)C]

[55] The compound, or pharmaceutically acceptable salt thereof, of any one of paragraphs [1] to [50], wherein B is optionally substituted 6-membered arylene.
[56] The compound, or pharmaceutically acceptable salt thereof, of paragraph [55], wherein B is 6-membered arylene.
[57] The compound, or pharmaceutically acceptable salt thereof, of paragraph [56], wherein B is:

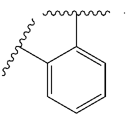 [SMILES: *c1ccccc1*]

[58] The compound, or pharmaceutically acceptable salt thereof, of any one of paragraphs [1] to [50], wherein B is absent.
[59] The compound, or pharmaceutically acceptable salt thereof, of any one of paragraphs [1] to [58], wherein R⁷ is methyl.
[60] The compound, or pharmaceutically acceptable salt thereof, of any one of paragraphs [1] to [59], wherein R⁸ is methyl.
[61] The compound, or pharmaceutically acceptable salt thereof, of any one of paragraphs [1] to [60], wherein the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A².
[62] The compound, or a pharmaceutically acceptable salt thereof, of any one of paragraphs [1] to [61], wherein the linker is acyclic.
[63] The compound, or a pharmaceutically acceptable salt thereof, of paragraph [62], wherein the linker has the structure of Formula IIa:

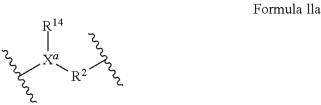

wherein X^{a} is absent or N;
R¹⁴ is absent, hydrogen, optionally substituted C₁-C₆ alkyl, or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present.
[64] The compound, or a pharmaceutically acceptable salt thereof, of paragraph [63], wherein the linker has the structure:

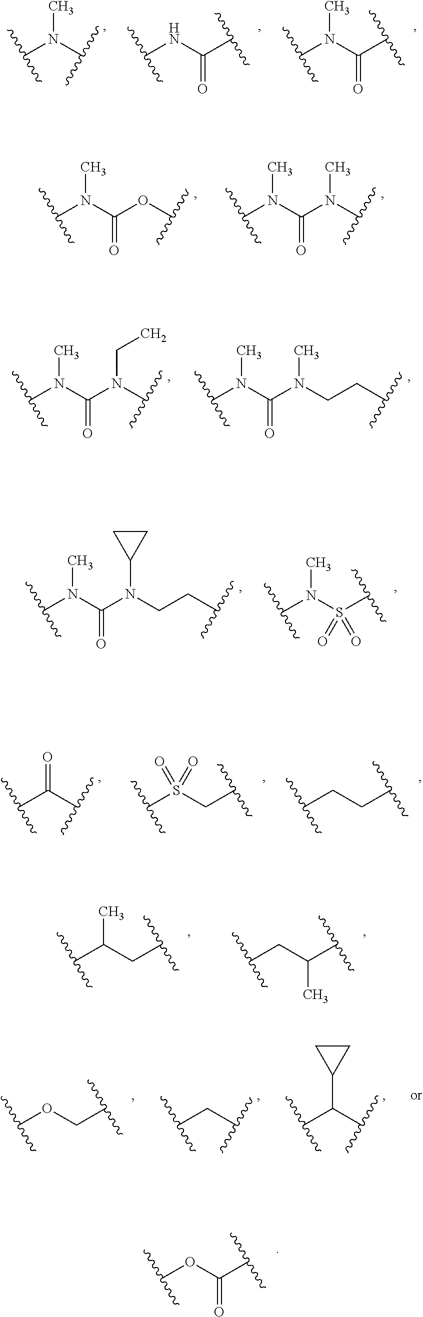

[65] The compound, or a pharmaceutically acceptable salt thereof, of paragraph [64], wherein the linker has the structure

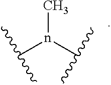

[66] The compound, or a pharmaceutically acceptable salt thereof, of any one of paragraphs [1] to [61], wherein the linker is or comprises a cyclic group.
[67] The compound, or a pharmaceutically acceptable salt thereof, of any one of paragraphs [1] to [61] or [66], wherein the linker has the structure of Formula IIb:

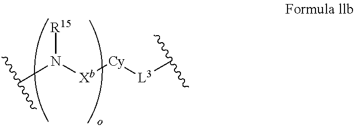

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene.
[68] The compound, or a pharmaceutically acceptable salt thereof, of paragraph [67], wherein the linker has the structure:

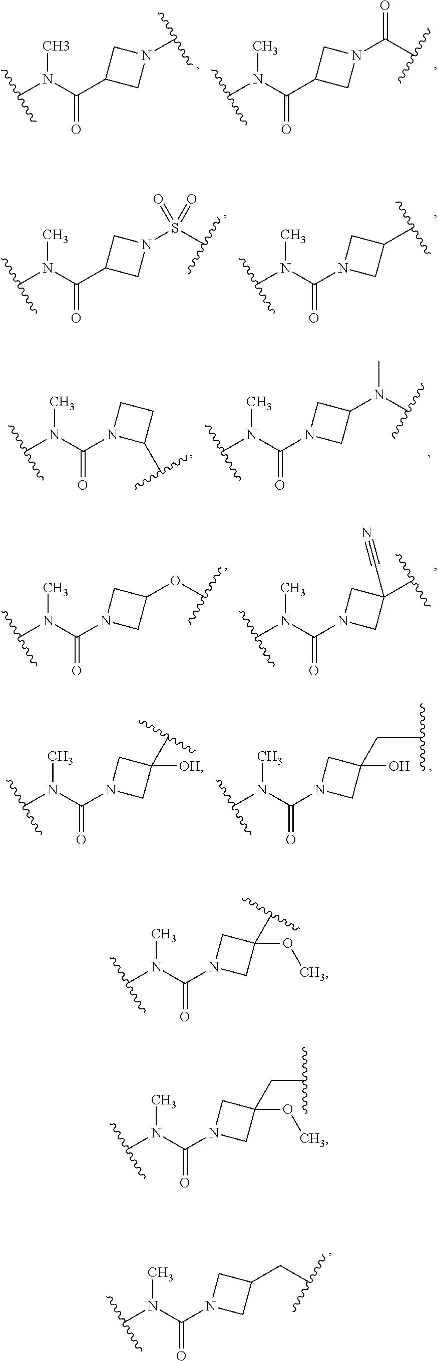

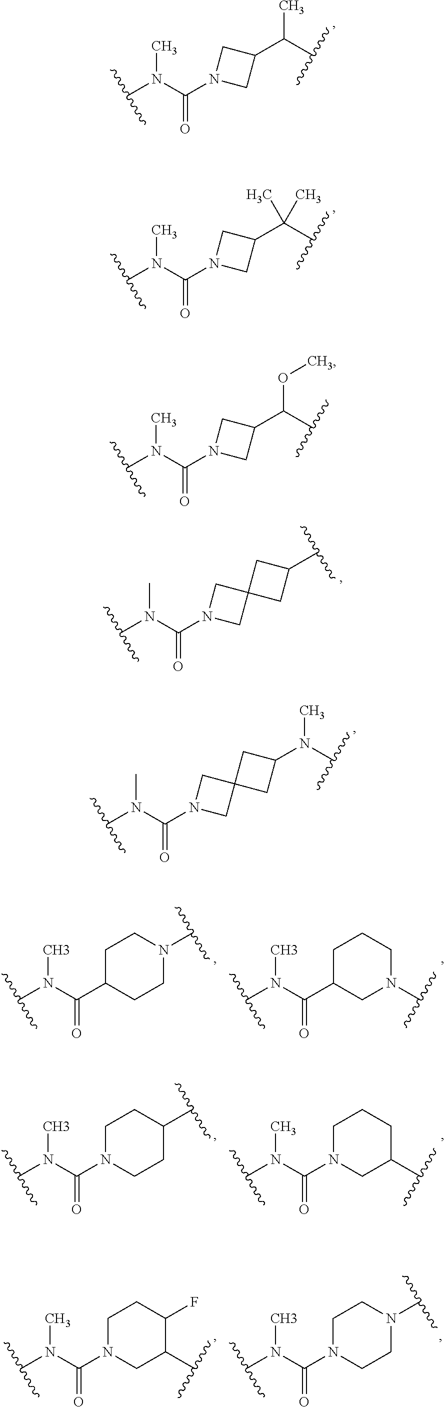

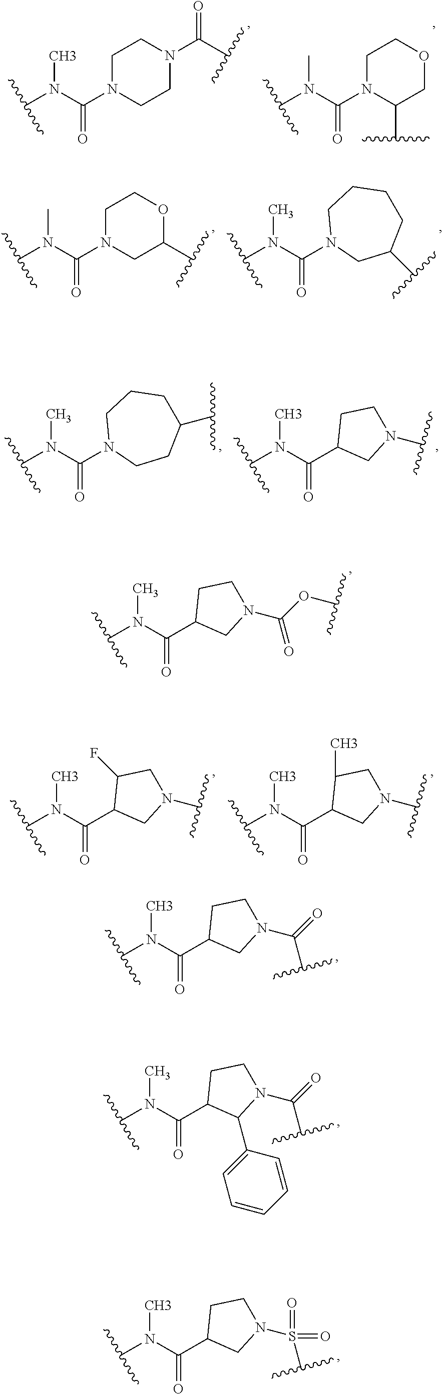

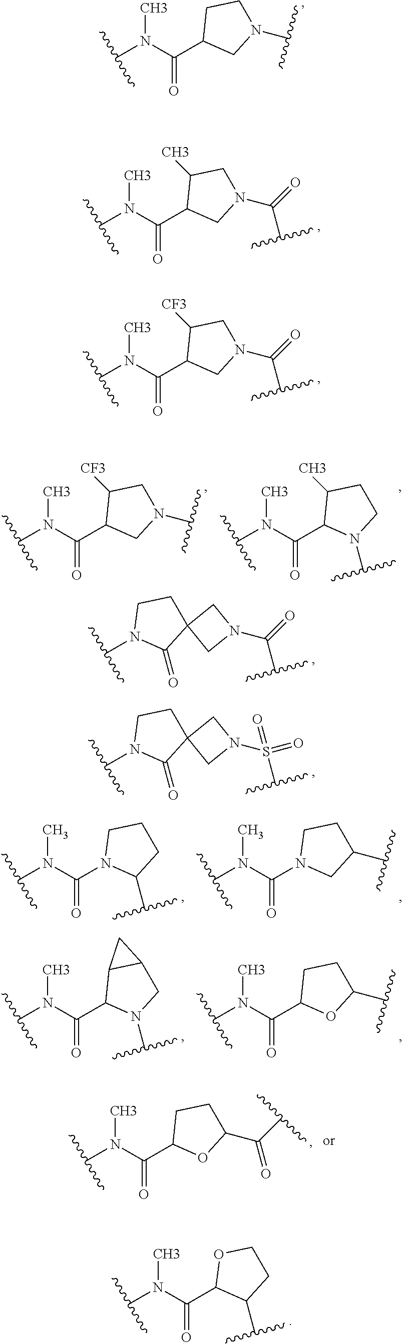

[69] The compound, or a pharmaceutically acceptable salt thereof, of any one of paragraphs [1] to [68], wherein W is hydrogen.
[70] The compound, or a pharmaceutically acceptable salt thereof, of any one of paragraphs [1] to [68], wherein W is optionally substituted amino.
[71] The compound, or a pharmaceutically acceptable salt thereof, of paragraph [70], wherein W is —NHCH₃ or —N(CH₃)₂.
[72] The compound, or a pharmaceutically acceptable salt thereof, of any one of paragraphs [1] to [68], wherein W is optionally substituted amido.
[73] The compound, or a pharmaceutically acceptable salt thereof, of paragraph [72], wherein W is

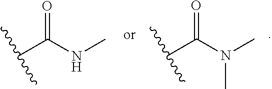

[74] The compound, or a pharmaceutically acceptable salt thereof, of any one of paragraphs [1] to [68], wherein W is optionally substituted C₁-C₄ alkoxy.
[75] The compound, or a pharmaceutically acceptable salt thereof, of paragraph [74], wherein W is methoxy or iso-propoxy.
[76] The compound, or a pharmaceutically acceptable salt thereof, of any one of paragraphs [1] to [68], wherein W is optionally substituted C₁-C₄ alkyl.
[77] The compound, or a pharmaceutically acceptable salt thereof, of paragraph [76], wherein W is methyl, ethyl, iso-propyl, tert-butyl, or benzyl.
[78] The compound, or a pharmaceutically acceptable salt thereof, of any one of paragraphs [1] to [68], wherein W is optionally substituted C₁-C₄ hydroxyalkyl.
[79] The compound, or a pharmaceutically acceptable salt thereof, of paragraph [78], wherein W is

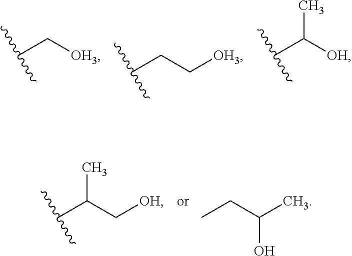

[80] The compound, or a pharmaceutically acceptable salt thereof, of any one of paragraphs [1] to [68], wherein W is optionally substituted C₁-C₄ aminoalkyl.
[81] The compound, or a pharmaceutically acceptable salt thereof, of paragraph [80], wherein W is

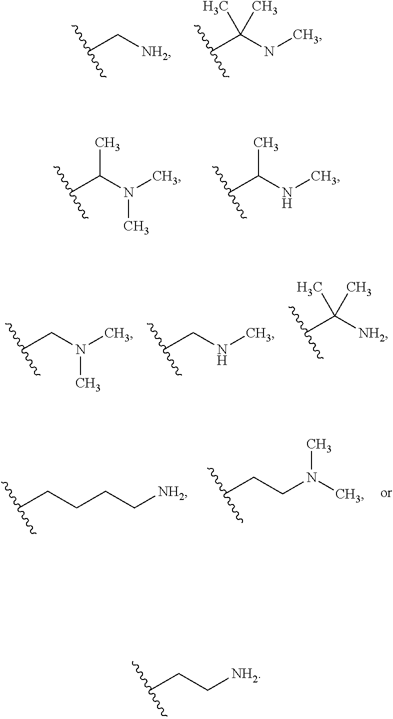

[82] The compound, or a pharmaceutically acceptable salt thereof, of any one of paragraphs [1] to [68], wherein W is optionally substituted C₁-C₄ haloalkyl.
[83] The compound, or a pharmaceutically acceptable salt thereof, of paragraph [82], wherein W is

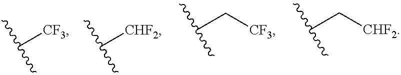

[84] The compound, or a pharmaceutically acceptable salt thereof, of any one of paragraphs [1] to [68], wherein W is optionally substituted C₁-C₄ guanidinoalkyl.
[85] The compound, or a pharmaceutically acceptable salt thereof, of paragraph [84], wherein W is

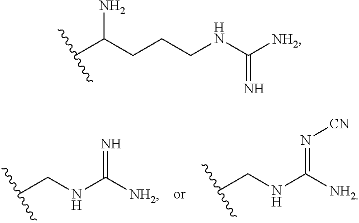 [SMILES: *C(N)CCCNC(=N)N ; *CNC(=N)N ; *CN/C(N)=N/C#N]

[86] The compound, or a pharmaceutically acceptable salt thereof, of any one of paragraphs [1] to [68], wherein W is C₀-C₄ alkyl optionally substituted 3 to 11-membered heterocycloalkyl.
[87] The compound, or a pharmaceutically acceptable salt thereof, of paragraph [86], wherein W is

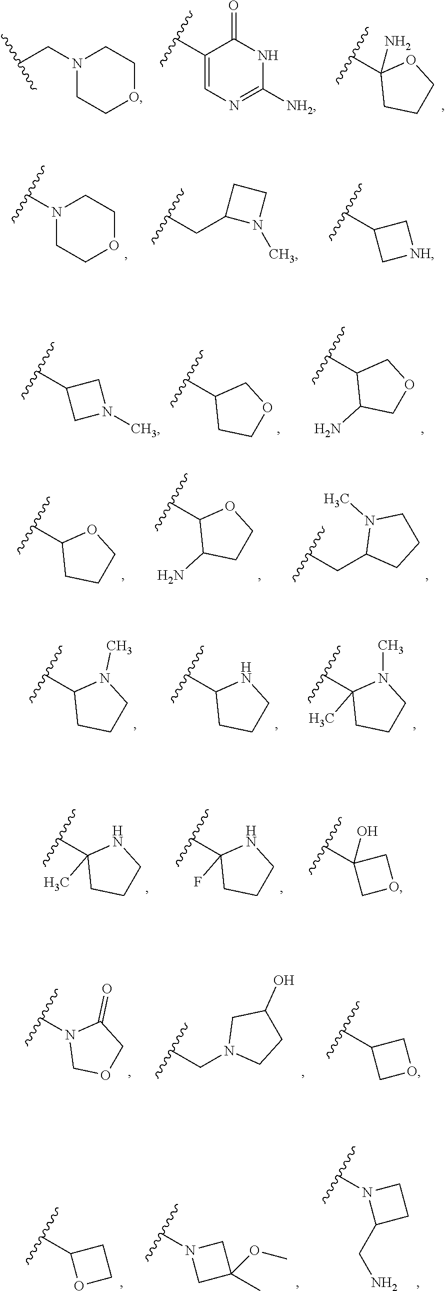

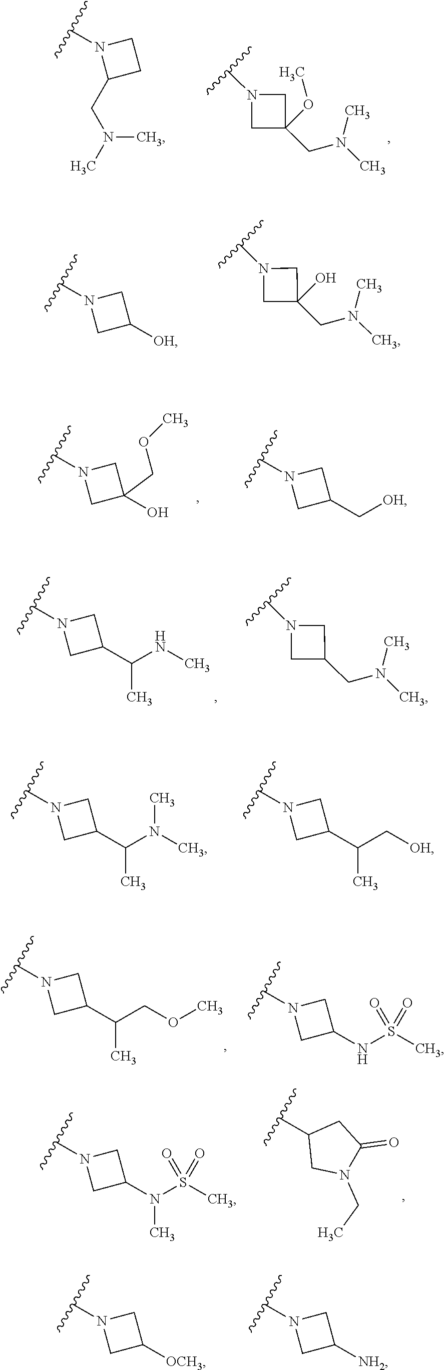

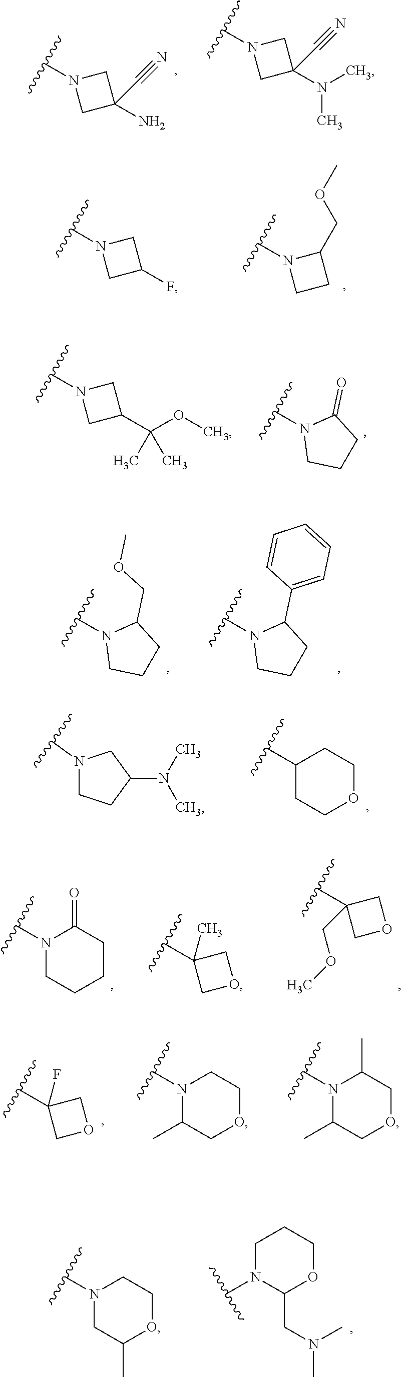

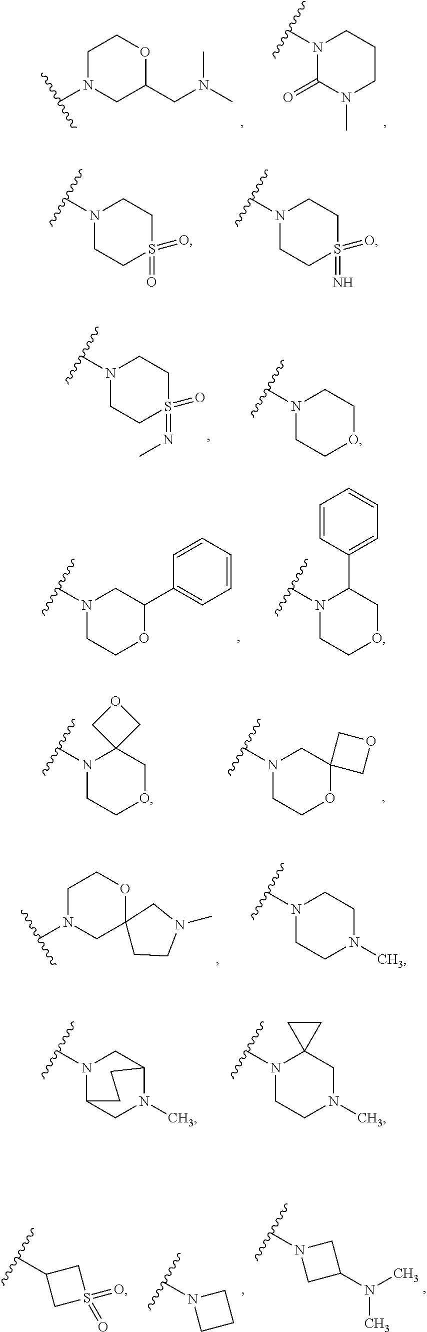

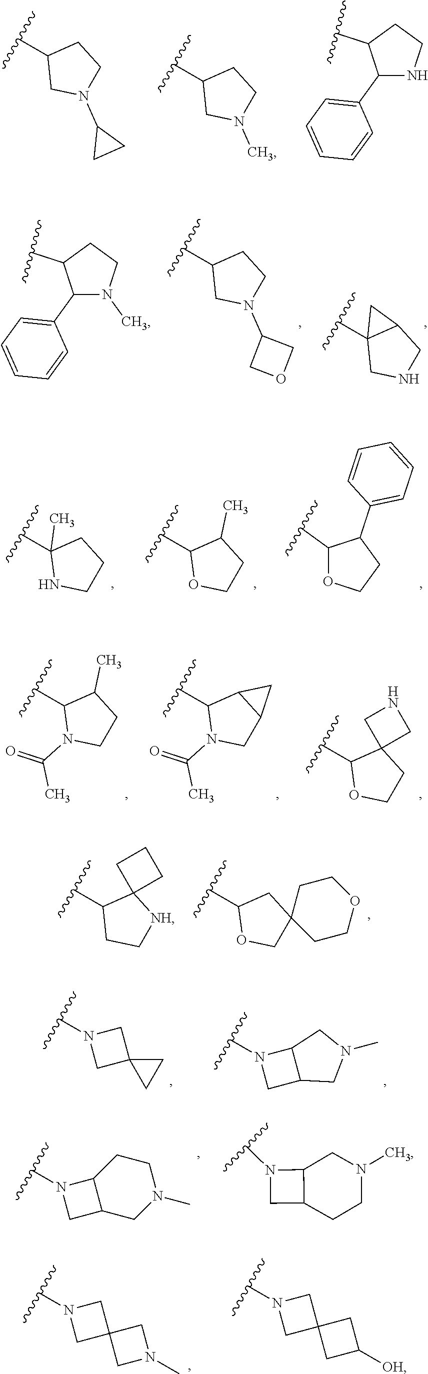

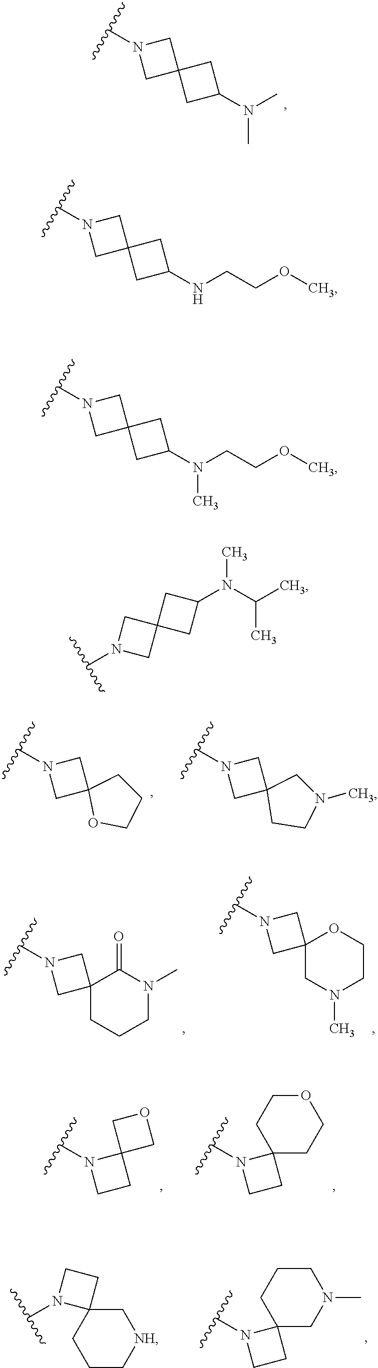

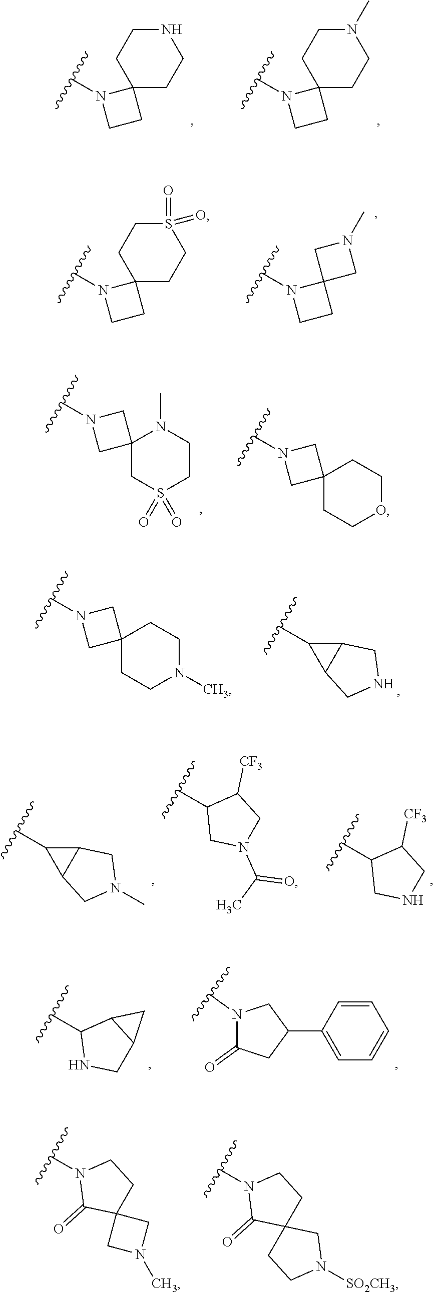

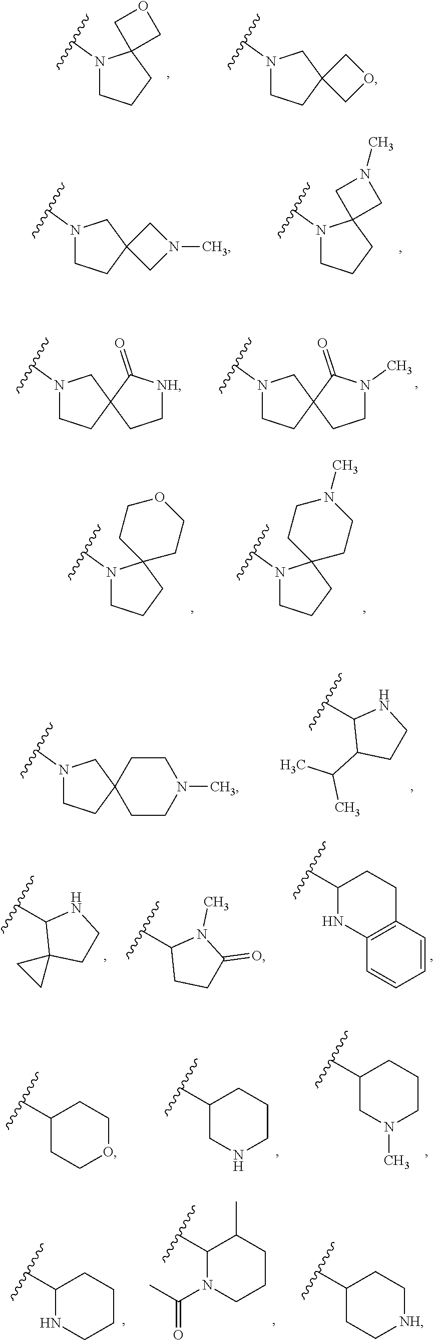

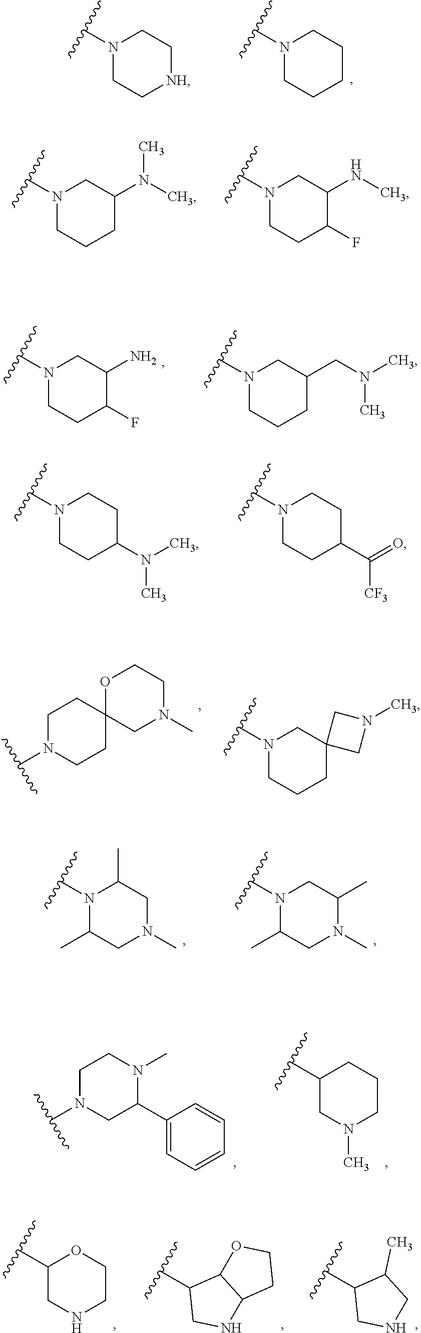

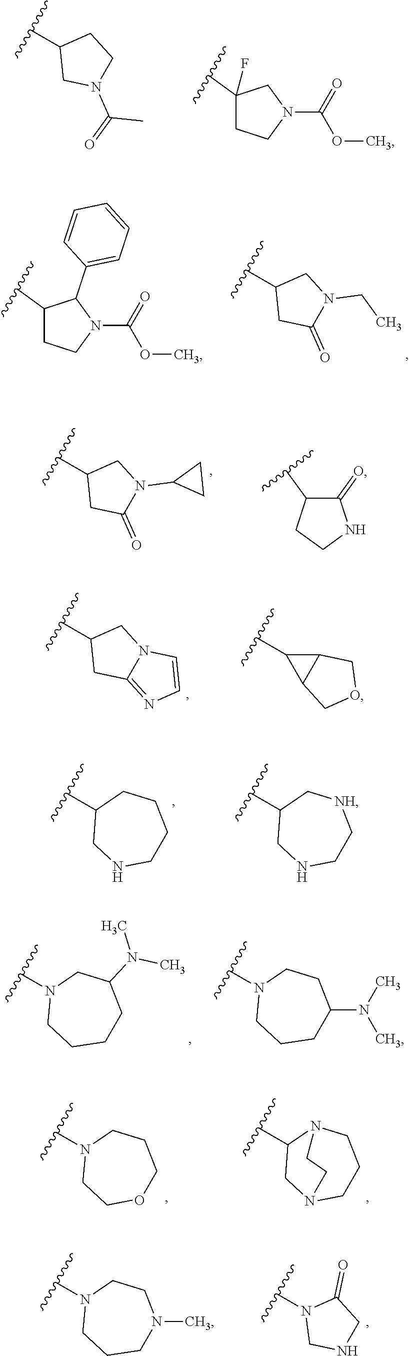

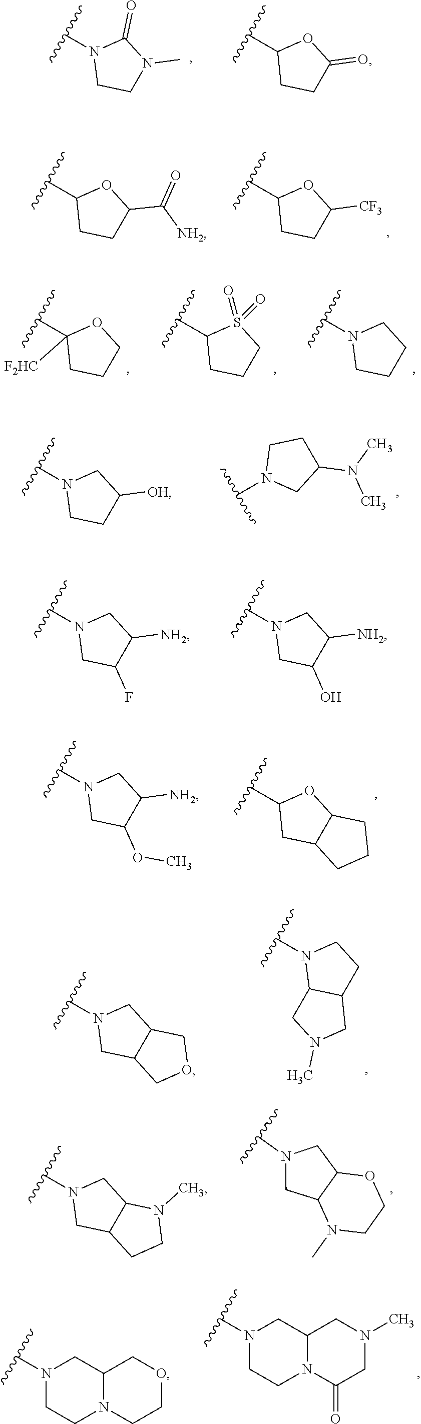

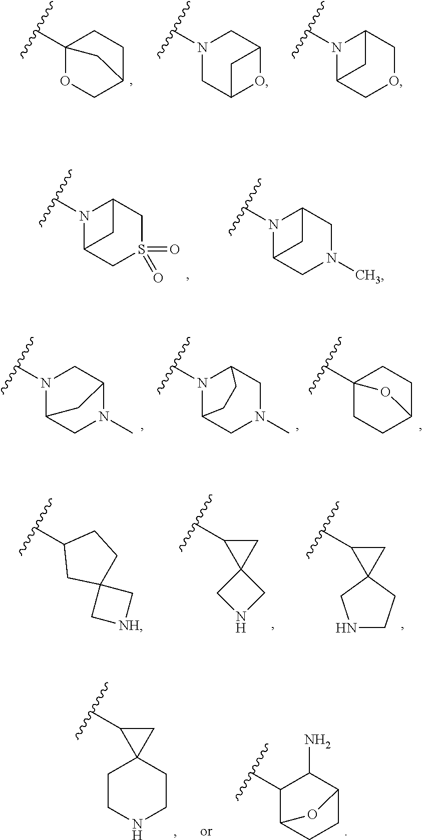

[88] The compound, or a pharmaceutically acceptable salt thereof, of any one of paragraphs [1] to [68], wherein W is optionally substituted 3 to 8-membered cycloalkyl.
[89] The compound, or a pharmaceutically acceptable salt thereof, of paragraph [88], wherein W is

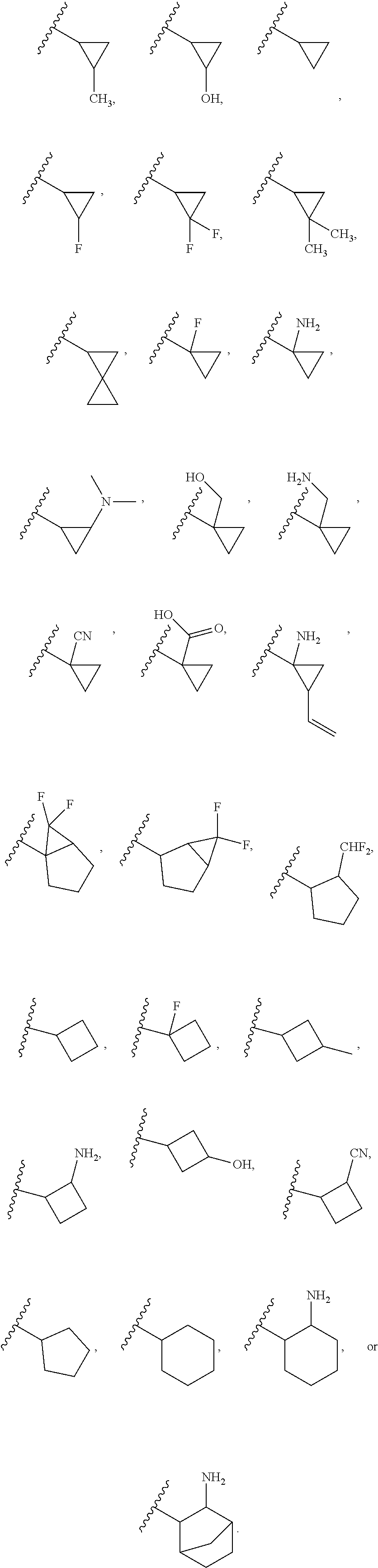

[90] The compound, or a pharmaceutically acceptable salt thereof, of any one of paragraphs [1] to [68], wherein W is optionally substituted 3 to 8-membered heteroaryl.
[91] The compound, or a pharmaceutically acceptable salt thereof, of paragraph [90], wherein W is

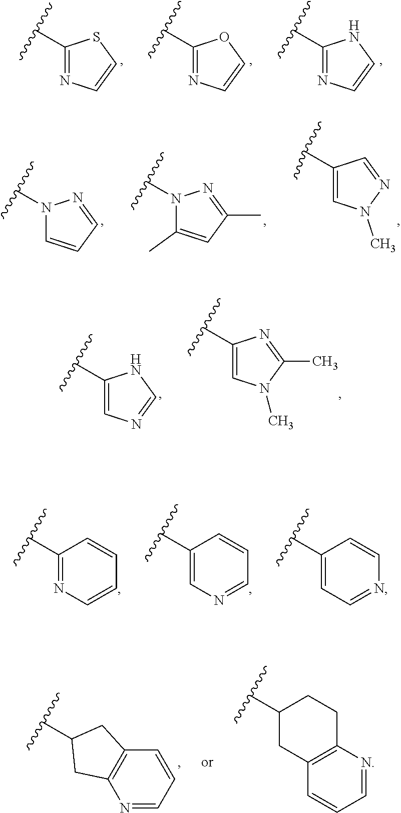

[92] The compound, or a pharmaceutically acceptable salt thereof, of any one of paragraphs [1] to [68], wherein W is optionally substituted 6- to 10-membered aryl.
[93] The compound, or a pharmaceutically acceptable salt thereof, or paragraph [92], wherein W is phenyl, 4-hydroxy-phenyl, or 2,4-methoxyphenyl.
[94] A compound, or a pharmaceutically acceptable salt thereof, of Table 1 or 2.
[95] A pharmaceutical composition comprising a compound, or a pharmaceutically acceptable salt thereof, of any one of paragraphs [1] to [94] and a pharmaceutically acceptable excipient.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:

| | |
|---|---|
| CH₂Cl₂, DCM | Methylene chloride, Dichloromethane |
| CH₃CN, MeCN | Acetonitrile |
| CuI | Copper (I) iodide |
| DIPEA | Diisopropylethyl amine |
| DMF | N, N-Dimethylformamide |
| EtOAc | Ethyl acetate |
| h | hour |
| H₂O | Water |
| HCl | Hydrochloric acid |
| K₃PO₄ | Potassium phosphate (tribasic) |
| MeOH | Methanol |
| Na₂SO₄ | Sodium sulfate |
| NMP | N-methyl pyrrolidone |
| Pd(dppf)Cl₂ | [1,1′-Bis(diphenylphosphino)ferrocene] |
| | dichloropalladium(II) |

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

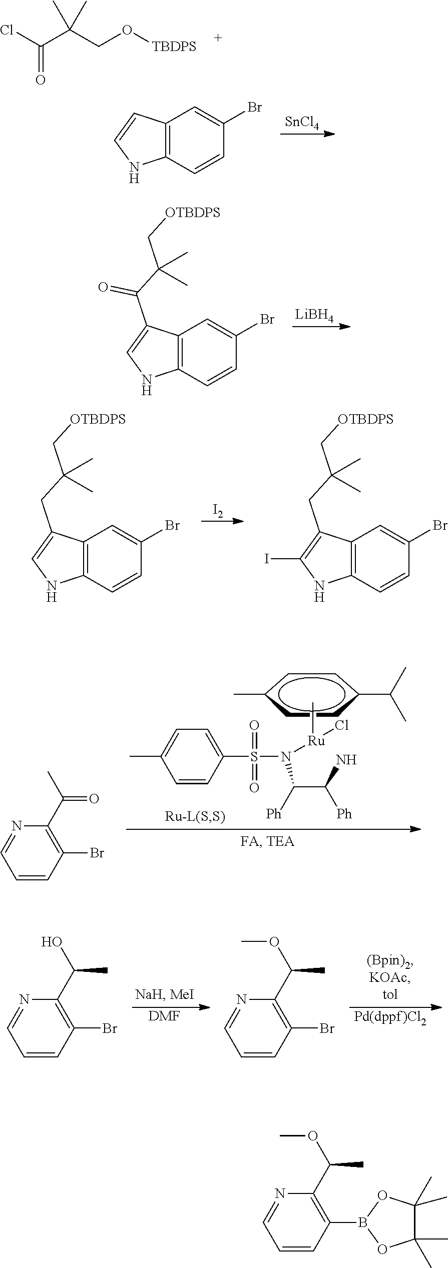

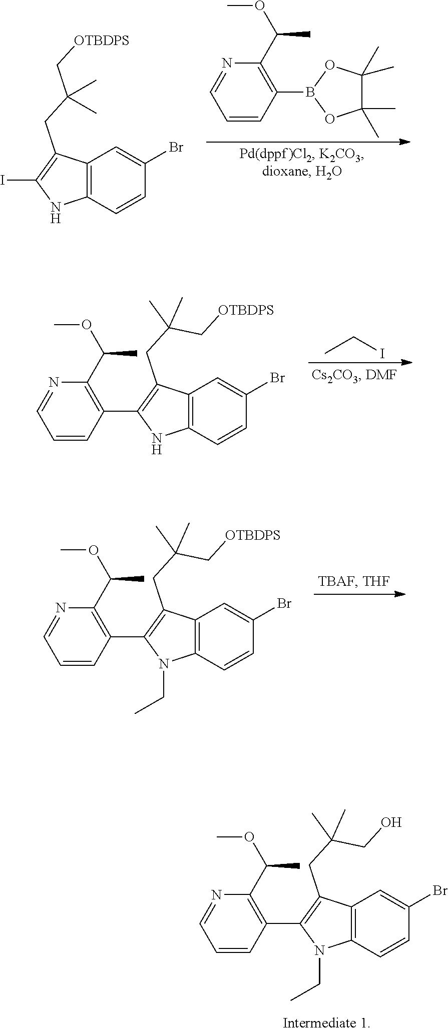

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na] calc'd for C₂₉H₃₂BrNO₂SiNa 556.1; found 556.3.

Step 2. To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ Was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C₂₉H₃₄BrNOSi 519.2; found 520.1; ¹H NMR (400 MHZ, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHZ, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl) ethanol (100 g, 74% yield) a an oil. LCMS (ESI): m/z [M+H] calc'd for C₇H₈BrNO 201.1; found 201.9.

Step 5. To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z [M+H] calc'd for C₈H₁₀BrNO 215.0; found 215.9.

Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis (pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl) pyridine (100 g, 63% yield) as a semi-solid. LCMS (ESI): m/z [M+H] calc'd for C₁₄H₂₂BNO₃ 263.2; found 264.1.

Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl) pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.

Step 8. To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to rt and stirred for 16 h then H₂O (4 L) added and the mixture extracted with EtOAc (3×1.5 L). The combined organic layers were washed with brine (2 ×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.

Step 9. To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI): m/z [M+H] calc'd for C₂₃H₂₉BrN₂O₂ 444.1; found 445.1.

### Intermediate 1. Alternative Synthesis Through Fisher Indole Route

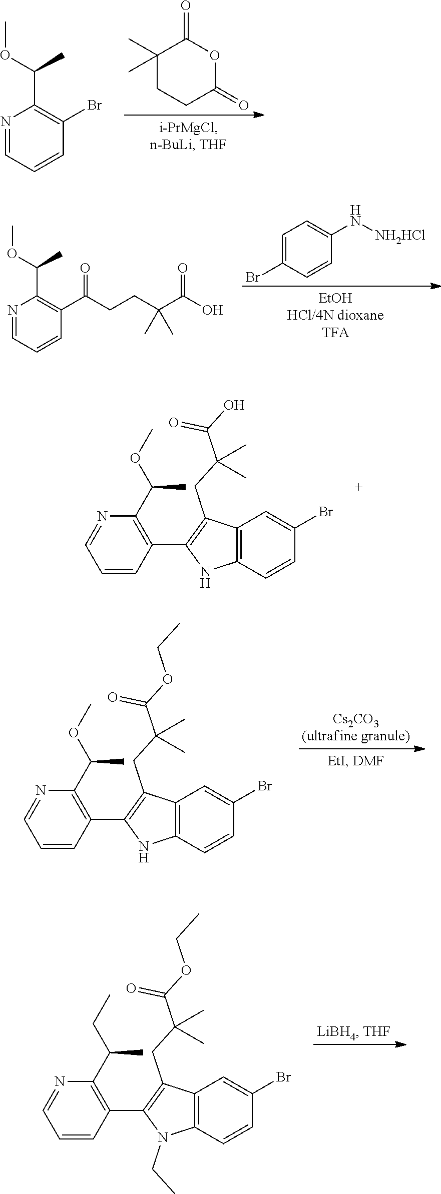

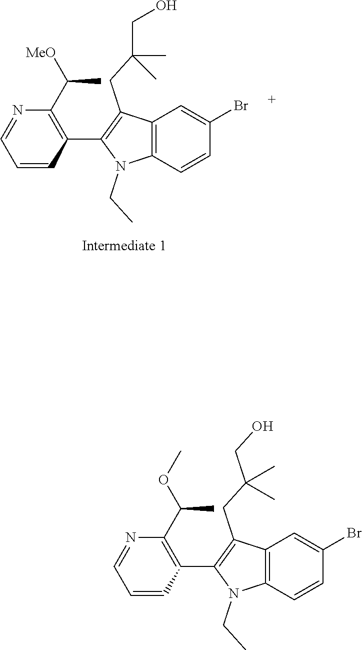

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.

Step 2. To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl) hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5, concentrated under reduced pressure and the residue adjusted to PH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

Step 3. To a mixture of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.

Step 4. To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH4 (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl) pyridin-3-yl)-1H-indol-3-yl)-2,2-dimethylpropan-1-ol (as single atropisomers) (60 g, 38% yield) and (40 g, 26% yield) both as solids. LCMS (ESI): m/z [M+H] calc'd for C₂₃H₂₉BrN₂O₂ 444.1; found 445.2.

### Intermediate 2 and Intermediate 4. Synthesis of(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate

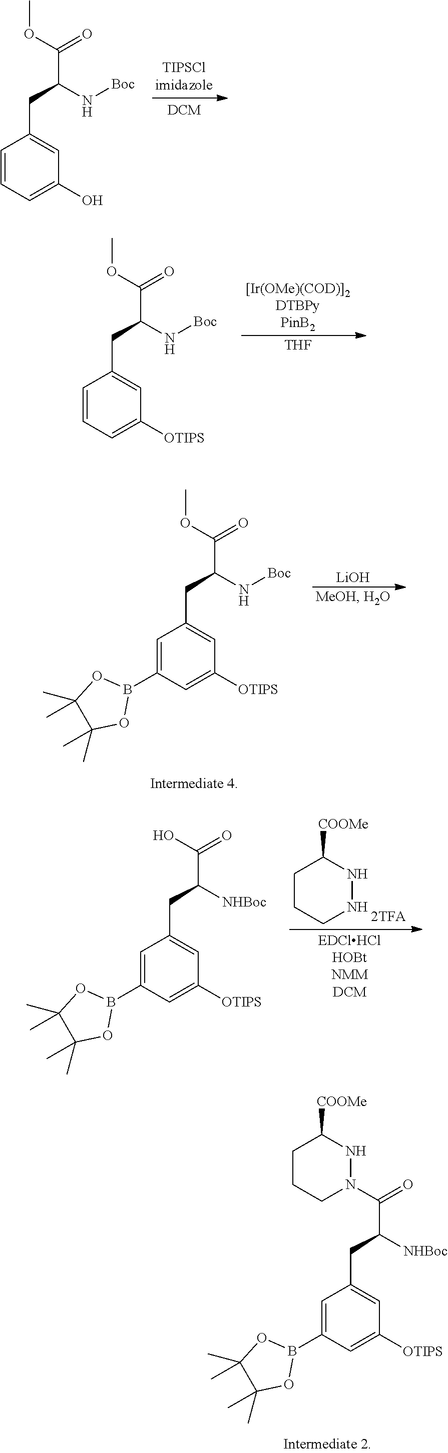

Step 1. To a mixture of(S)-methyl 2-(tert-butoxycarbonylamino)-3-(3-hydroxyphenyl) propanoate (10.0 g, 33.9 mmol) in DCM (100 mL) was added imidazole (4.6 g, 67.8 mmol) and TIPSCl (7.8 g, 40.7 mmol). The mixture was stirred at rt overnight then diluted with DCM (200 mL) and washed with H₂O (150 mL×3). The organic layer was dried over anhydrous Na₂SO₄, filtered, concentrated under reduced pressure and the residue was purified by silica gel column chromatography to give (S)-methyl 2-(tert-butoxycarbonylamino)-3-(3-(triisopropylsilyloxy)phenyl)-propanoate (15.0 g, 98% yield) as an oil. LCMS (ESI): m/z [M+Na] calc'd for C₂₄H₄₁NO₅SiNa 474.3; found 474.2.

Step 2. A mixture of(S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol) and 4-tert-butyl-2-(4-tert-butyl-2-pyridyl) pyridine (1.3 g, 5.0 mmol) was purged with Ar (×3), then THF (75 mL) was added and the mixture placed under an atmosphere of Ar and sealed. The mixture was heated to 80° C. and stirred for 16 h, concentrated under reduced pressure and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI): m/z [M+Na] calc'd for C₃₀H₅₂BNO₇SiNa 600.4; found 600.4; ¹H NMR (300 MHz, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

Step 3. To a mixture of triisopropylsilyl(S)-2-((tert-butoxycarbonyl) amino)-3-(3-(4,4,5,5-tetramethyl-1,3,2-dioxaborolan-2-yl)-5-((triisopropylsilyl)oxy) phenyl) propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to PH ˜5 with 1M HCl and extracted with EtOAc (250 mL×2). The combined organic layers were washed with brine (100 mL×3), dried over anhydrous Na₂SO₄, filtered and the filtrate concentrated under reduced pressure to give (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (3.7 g, 95% yield), which was used directly in the next step without further purification. LCMS (ESI): m/z [M+NH4] calc'd for C₂₉H₅₀BNO₇SiNH₄ 581.4; found 581.4.

Step 4. To a mixture of methyl(S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (24 g, 42.6 mmol) in DCM (50 mL) then HOBt (1.21 g, 9.0 mmol) and EDCl HCl salt (12.9 g, 67.6 mmol). The mixture was warmed to rt and stirred for 16 h, then diluted with DCM (200 mL) and washed with H₂O (3×150 mL). The organic layer was dried over anhydrous Na₂SO, filtered, the filtrate concentrated under reduced pressure and the residue was purified by silica gel column chromatography to give methyl(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate (22 g, 71% yield) as an oil. LCMS (ESI): m/z [M+H] calc'd for C₃₅H₆₀BN₃O₈Si 689.4; found 690.5.

### Intermediate 3. Synthesis of(S)-tert-butyl 3-methyl-2-((S)—N-methylpyrrolidine-3-carboxamido) butanoate

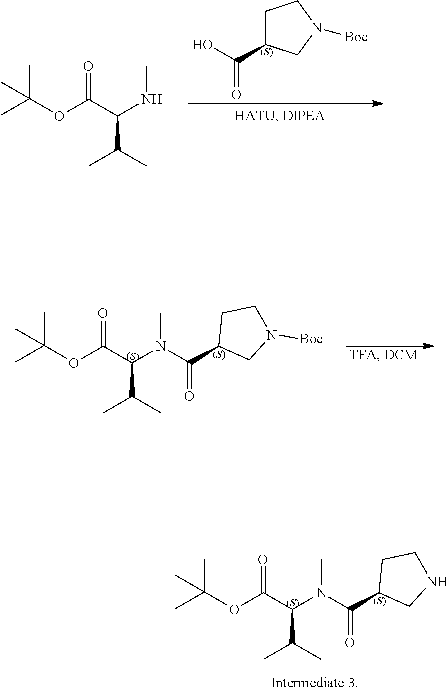

Step 1. To a mixture of(S)-1-(tert-butoxycarbonyl) pyrrolidine-3-carboxylic acid (2.2 g, 10.2 mmol) in DMF (10 mL) at rt was added HATU (7.8 g, 20.4 mmol) and DIPEA (5 mL). After stirring at rt for 10 min, tert-butyl methyl-L-valinate (3.8 g, 20.4 mmol) in DMF (10 mL) was added. The mixture was stirred at rt for 3 h, then diluted with DCM (40 mL) and H₂O (30 mL). The aqueous and organic layers were separated, and the organic layer was washed with H₂O (3×30 mL), brine (30 mL), dried over anhydrous Na₂SO₄ and filtered. The filtrate was concentrated under reduced pressure and the residue was purified by silica gel column chromatography to give (S)-tert-butyl 3-(((S)-1-(tert-butoxy)-3-methyl-1-oxobutan-2-yl) (methyl) carbamoyl) pyrrolidine-1-carboxylate (3.2 g, 82% yield) as an oil. LCMS (ESI): m/z [M+Na] calc'd for C₂₀H₃₆N₂O₅Na 407.3; found 407.2.

Step 2. A mixture of(S)-tert-butyl 3-(((S)-1-(tert-butoxy)-3-methyl-1-oxobutan-2-yl) (methyl) carbamoyl) pyrrolidine-1-carboxylate (3.2 g, 8.4 mmol) in DCM (13 mL) and TFA (1.05 g, 9.2 mmol) was stirred at rt for 5 h. The mixture was concentrated under reduced pressure to give (S)-tert-butyl 3-methyl-2-((S)—N-methylpyrrolidine-3-carboxamido) butanoate (2.0 g, 4% yield) as an oil. LCMS (ESI): m/z [M+H] calc'd for C₁₅H₂₈N₂O₃ 284.2; found 285.2.

### Intermediate 5. Synthesis of tert-butyl ((63S,4S)-11-ethyl-12-(2-((S)-1-methoxyethyl) pyridin-3-yl)-10,10-dimethyl-5,7-dioxo-2⁵-((triisopropylsilyl)oxy)-61,62,63,64,65,66-hexahydro-11H-8-oxa-1 (5,3)-indola-6 (1,3)-pyridazina-2 (1,3)-benzenacycloundecaphane-4-yl) carbamate

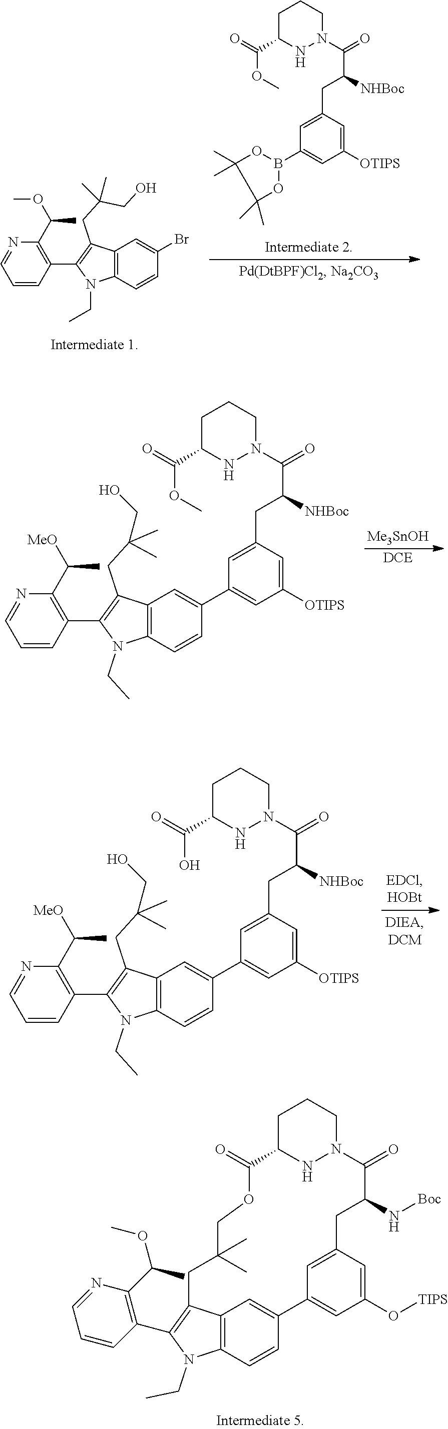

Step 1. To a stirred mixture of 3-(5-bromo-1-ethyl-2-[2-[(1S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at rt under an atmosphere of Ar was added Na₂CO₃ (17.9 g, 168.4 mmol), Pd(DtBPF)Cl₂ (4.39 g, 6.7 mmol) and H₂O (150.00 mL) in portions. The mixture was heated to 85° C. and stirred for 3 h, cooled, diluted with H₂O (2 L) and extracted with EtOAc (3×1 L). The combined organic layers were washed with brine (2×500 mL), dried over anhydrous Na₂SO₄ and filtered. The filtrate was concentrated under reduced pressure and the residue was purified by silica gel column chromatography to give methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI): m/z [M+H] calc'd for C₅₂H₇₇N₅O₈Si 927.6; found 928.8.

Step 2. To a stirred mixture of methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at rt was added trimethyltin hydroxide (48.7 g, 269 mmol) in portion. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI): m/z [M+H] calc'd for C₅₁H₇₅N₅O₈Si 913.5; found 914.6.

Step 3. To a stirred mixture of (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (297 g, 2.3 mol), HOBT (51.7 g, 383 mmol) and EDCl (411 g, 2.1 mol) in portions. The mixture was warmed to rt and stirred for 16 h, then diluted with DCM (1 L), washed with brine (3×1 L), dried over anhydrous Na₂SO₄ and filtered. The filtrate was concentrated under reduced pressure and the residue was purified by silica gel column chromatography to give tert-butyl ((63S,4S)-11-ethyl-12-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (36 g, 42% yield) as a solid. LCMS (ESI): m/z [M+H] calc'd for C₅₁H₇₃N₅O₇Si 895.5; found 896.5.

### Intermediate 6. Synthesis of tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]carbamate

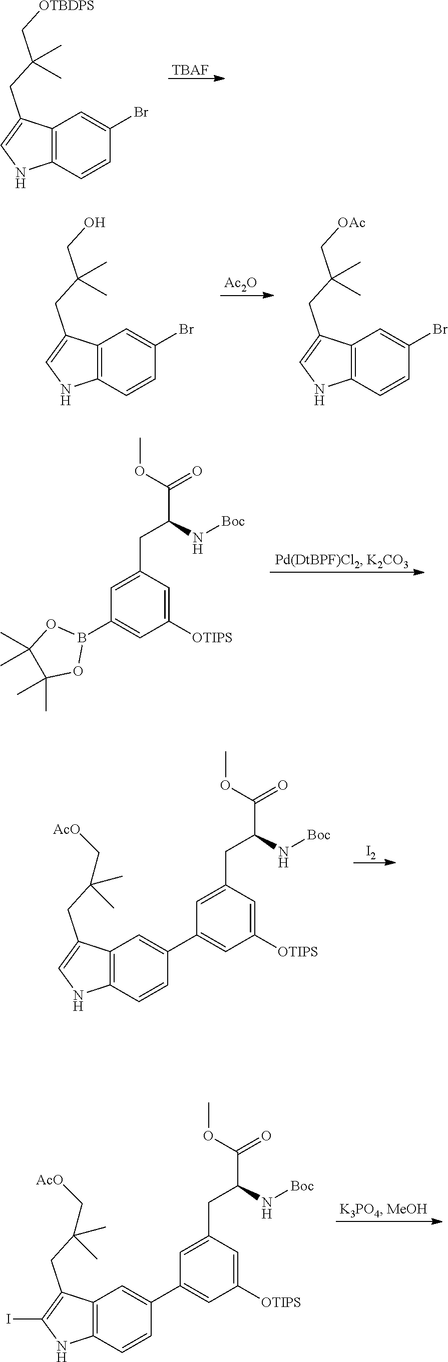

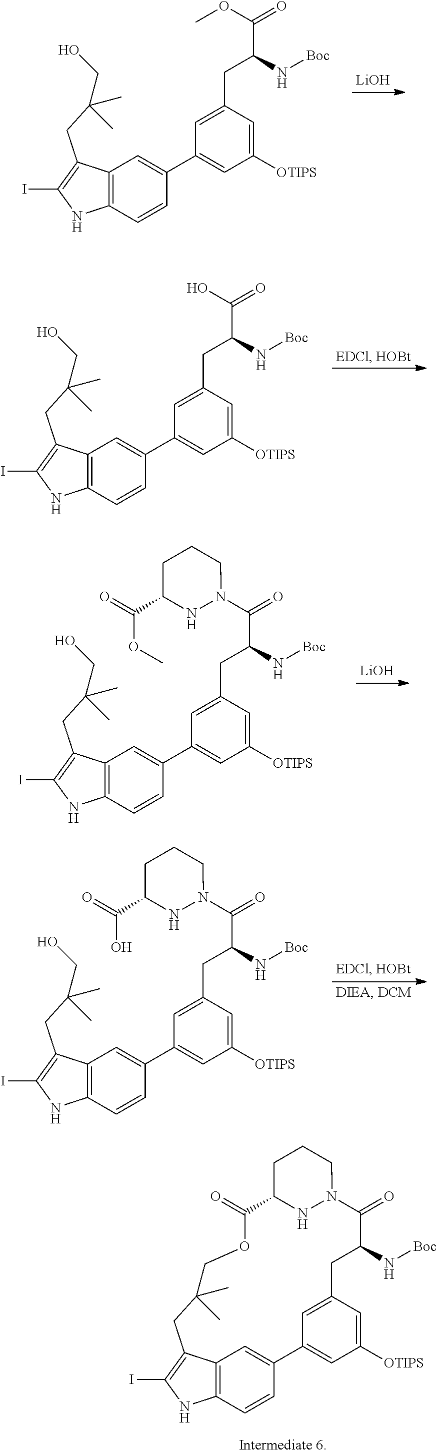

Step 1. This reaction was undertaken on 5-batches in parallel on the scale illustrated below.

Into a 2 L round-bottom flasks each were added 5-bromo-3-[3-[(tert-butyldiphenylsilyl)oxy]-2,2-dimethylpropyl]-1H-indole (100 g, 192 mmol) and TBAF (301.4 g, 1.15 mol) in THF (1.15 L) at rt. The resulting mixture was heated to 50° C. and stirred for 16 h, then the mixture was concentrated under reduced pressure. The combined residues were diluted with H₂O (5 L) and extracted with EtOAc (3×2 L). The combined organic layers were washed with brine (2×1.5 L), dried over anhydrous Na₂SO₄ and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI): m/z [M+H] calc'd for C₁₃H₁₆BrNO 281.0 and 283.0; found 282.1 and 284.1.

Step 2. This reaction was undertaken on 2-batches in parallel on the scale illustrated below.

To a stirred mixture of 3-(5-bromo-1H-indol-3-yl)-2,2-dimethylpropan-1-ol (135 g, 478 mmol) and TEA (145.2 g, 1.44 mol) in DCM (1.3 L) at 0° C. under an atmosphere of N₂ was added Ac₂O (73.3 g, 718 mmol) and DMAP (4.68 g, 38.3 mmol) in portions. The resulting mixture was stirred for 10 min at 0° C., then washed with H₂O (3×2 L). The organic layers from each experiment were combined and washed with brine (2×1 L), dried over anhydrous Na₂SO₄ and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. ¹H NMR (400 MHZ, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

Step 3. This reaction was undertaken on 4-batches in parallel on the scale illustrated below.

Into a 2 L round-bottom flasks were added methyl (2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol) and Pd(DtBPF)Cl₂ (7.05 g, 10.8 mmol) at rt under an atmosphere of Ar. The resulting mixture was heated to 65° C. and stirred for 2 h, then diluted with H₂O (10 L) and extracted with EtOAc (3×3 L). The combined organic layers were washed with brine (2×2 L), dried over anhydrous Na₂SO₄ and filtered. The filtrate was concentrated under reduced pressure and the residue was purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (500 g, 74% yield) as an oil. LCMS (ESI): m/z [M+Na] calc'd for C₃₉H₅₈N₂O₇SiNa 717.4; found 717.3.

Step 4. This reaction was undertaken on 3-batchs' in parallel on the scale illustrated below. To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (150 g, 216 mmol) and NaHCO₃ (21.76 g, 259 mmol) in THF (1.5 L) was added AgOTf (66.5 g, 259 mmol) in THE dropwise at 0° C. under an atmosphere of nitrogen. 12 (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄ and filtered. The filtrate was concentrated under reduced pressure and the residue was purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (420 g, 71% yield) as an oil. LCMS (ESI): m/z [M+Na] calc'd for C₃₉H₅₇IN₂O₇SiNa, 843.3; found 842.9.

Step 5. This reaction was undertaken on 3-batches in parallel on the scale illustrated below.

To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (140 g, 171 mmol), MeOH (1.4 L) and K3PO4 (108.6 g, 512 mmol) at 0° C. The mixture was warmed to rt and stirred for 1 h, then the combined experiments were diluted with H₂O (9 L) and extracted with EtOAc (3×3 L). The combined organic layers were washed with brine (2×2 L), dried over anhydrous Na₂SO₄, filtered and the filtrate was concentrated under reduced pressure to give methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI): m/z [M+Na] calc'd for C₃₇H₅₅IN₂O₆SiNa 801.3; found 801.6.

Step 6. This reaction was undertaken on 3-batches in parallel on the scale illustrated below.

To a stirred mixture of methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to rt and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered and the filtrate was concentrated under reduced pressure to give (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI): m/z [M+Na] calc'd for C₃₆H₅₃IN₂O₆SiNa 787.3; found 787.6.

Step 7. To a stirred mixture of (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCl (170 g, 889 mmol), HOBT (12.0 g, 88.9 mmol) portionwise at 0° C. The mixture was warmed to rt and stirred for 16 h, then washed with H₂O (3×2.5 L), brine (2×1 L), dried over anhydrous Na₂SO₄ and filtered. The filtrate was concentrated under reduced pressure and the residue was purified by column chromatography to give methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI): m/z [M+H] calc'd for C₄₂H₆₃IN₄O₇Si 890.4; found 890.8.

Step 8. This reaction was undertaken on 3-batches in parallel on the scale illustrated below.

To a stirred mixture of methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) each added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl and the combined experiments extracted with EtOAc (3×2 L). The combined organic layers were washed with brine (2×1.5 L), dried over anhydrous Na₂SO₄, filtered and the filtrate was concentrated under reduced pressure to give (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI): m/z [M+H] calc'd for C₄₁H₆₁IN₄O₇Si 876.3; found 877.6.

Step 9. This reaction was undertaken on 2-batches in parallel on the scale illustrated below.

To a stirred mixture of (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (265 g, 2.05 mol), EDCl (394 g, 2.05 mol), HOBT (37 g, 274 mmol) in portions at 0° C. under an atmosphere of N₂. The mixture was warmed to rt and stirred overnight, then the combined experiments were washed with H₂O (3×6 L), brine (2×6 L), dried over anhydrous Na₂SO₄ and filtered. After filtration, the filtrate was concentrated under reduced pressure. The filtrate was concentrated under reduced pressure and the residue was purified by column chromatography to give tert-butyl N-[(8S,14S)-21-iodo-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]]nonacosa-1 (26),2,4,6 (29),20,23 (27),24-heptaen-8-yl]carbamate (140 g, 50% yield) as a solid. LCMS (ESI): m/z [M+H] calc'd for C₄₁H₅₉IN₄O₆Si 858.9; found 858.3.

### Intermediate 7. Synthesis of (63S,45)-4-amino-11-ethyl-25-hydroxy-1²-(4-(methoxymethyl)pyridin-3-yl)-10,10-dimethyl-6¹,6²,6³,6⁴,6⁵,6⁶-hexahydro-11H-8-oxa-1 (5,3)-indola-6 (1,3)-pyridazina-2 (1,3)-benzenacycloundecaphane-5,7-dione

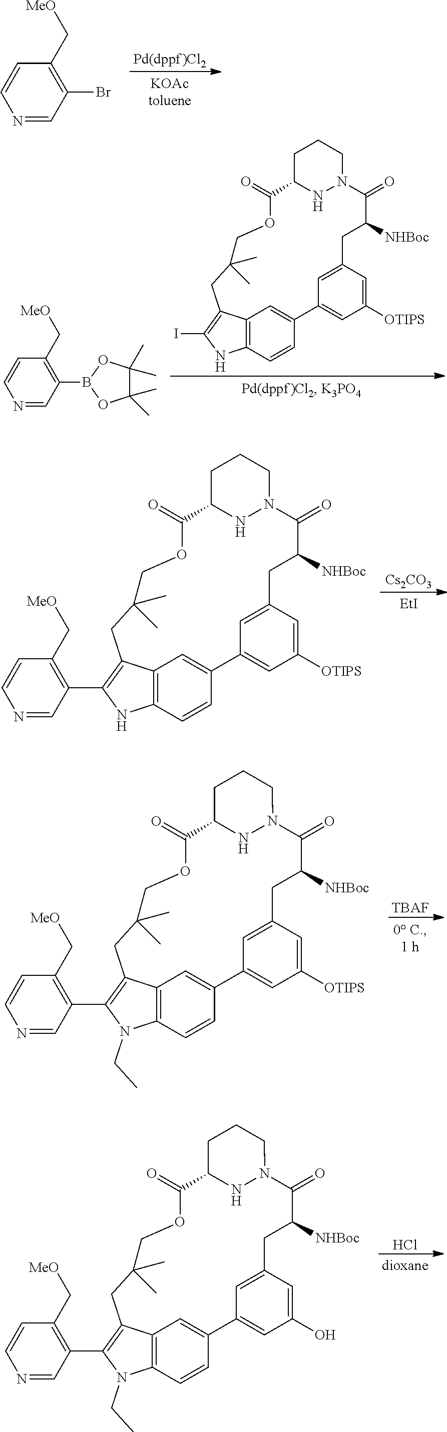

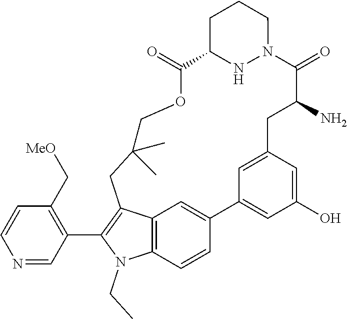 [SMILES: CCn1c(-c2cnccc2COC)c2c3cc(ccc31)-c1cc(O)cc(c1)C[C@H](N)C(=O)N1CCC[C@H](N1)C(=O)OCC(C)(C)C2]

Step 1. To a mixture of 3-bromo-4-(methoxymethyl)pyridine (1.00 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at rt under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl) pyridine, which was used directly in the next step directly without further purification. LCMS (ESI): m/z [M+H] calc'd for C₁₃H₂₀BNO₃ 249.2; found 250.3.

Step 2. To a mixture of 4-(methoxymethyl)-3-(4,4,5,5-tetramethyl-1,3,2-dioxaborolan-2-yl) pyridine (290 mg, 1.16 mmol), K₃PO₄ (371 mg, 1.75 mmol) and tert-butyl N-[(8S,14S)-21-iodo-18,18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (500 mg, 0.58 mmol) in 1,4-dioxane (5 mL) and H₂O (1 mL) at rt under an atmosphere of Ar was added Pd(dppf)Cl₂ (43 mg, 0.06 mmol). The mixture was heated to 70° C. and stirred for 2 h, then H₂O added and the mixture extracted with EtOAc (2×10 mL). The combined organic layers were washed with brine (10 mL), dried over anhydrous Na₂SO₄ and filtered. The filtrate was concentrated under reduced pressure and the residue was purified by silica gel column chromatography to give tert-butyl N-[(8S, 14S)-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]carbamate (370 mg,74% yield) as a foam. LCMS (ESI): m/z [M+H] calc'd for C₄₈H₆₇N₅O₇Si 853.6; found 854.6.

Step 3. A mixture of tert-butyl N-[(8S,14S)-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26), 2,4,6 (29),20,23 (27),24-heptaen-8-yl]carbamate (350 mg, 0.41 mmol), Cs₂CO₃ (267 mg, 0.82 mmol) and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄ and filtered. The filtrate was concentrated under reduced pressure and the residue was purified by silica gel column chromatography to give tert-butyl N-[(8S,14S)-22-ethyl-21-[4-(methoxymethyl)pyridin-3-yl]-18, 18-dimethyl-9,15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]carbamate (350 mg, 97% yield) as an oil. LCMS (ESI): m/z [M+H] calc'd for C₅₀H₇₁N₅O₇Si 881.5; found 882.6.

Step 4. A mixture of tert-butyl N-[(8S, 14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (350 mg, 0.4 mmol) and 1M TBAF in THF (0.48 mL, 0.480 mmol) in THF (3 mL) at 0° C. under an atmosphere of Ar was stirred for 1 h. The mixture was concentrated under reduced pressure and the residue was purified by silica gel column chromatography to give tert-butyl N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]carbamate (230 mg, 80% yield) as an oil. LCMS (ESI): m/z [M+H] calc'd for C₄₁H₅₁N₅O₇ 725.4; found 726.6.

Step 5. To a mixture of tert-butyl N-[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to rt and was stirred overnight, then concentrated under reduced pressure to give (6³S,4^{S})-4-amino-11-ethyl-25-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (200 mg). LCMS (ESI): m/z [M+H] calc'd for C₃₆H₄₃N₅O₅ 625.3; found 626.5.

### Intermediate 8. Synthesis of tert-butyl ((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

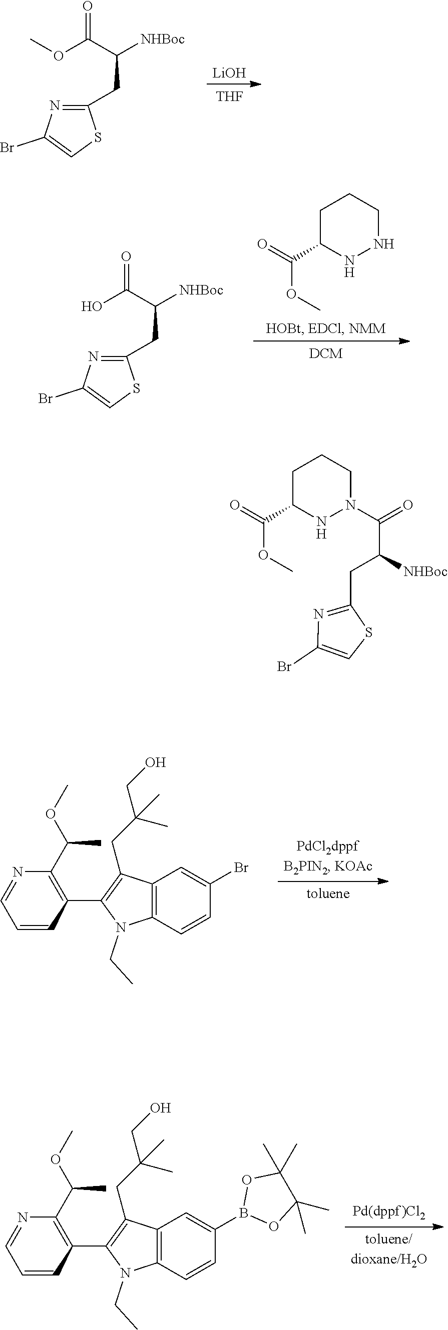

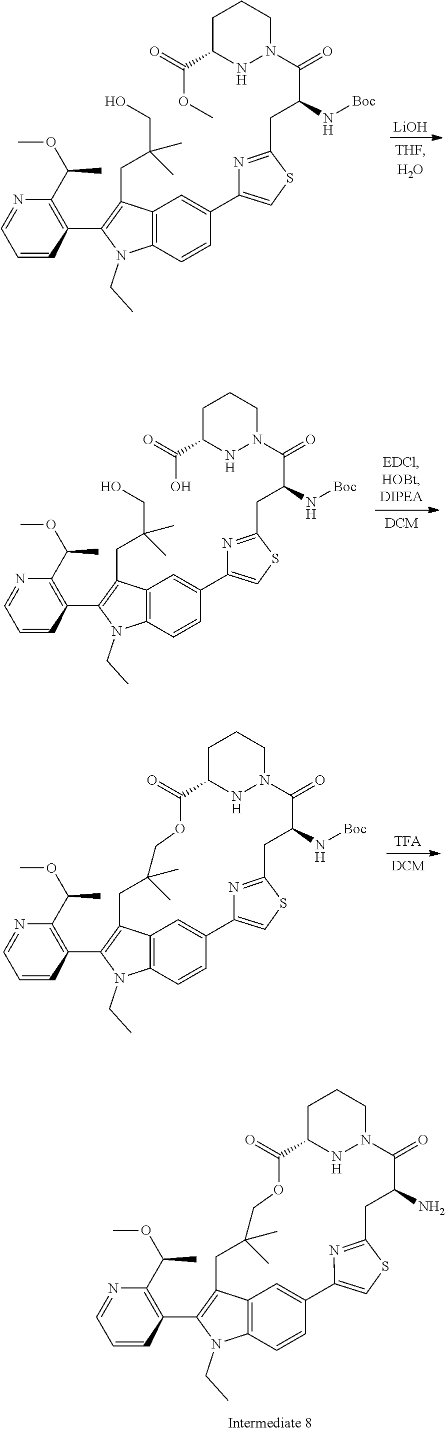

Step 1. To a solution of methyl (2S)-3-(4-bromo-1,3-thiazol-2-yl)-2-[(tert-butoxycarbonyl) amino]propanoate (110 g, 301.2 mmol) in THF (500 mL) and H₂O (200 mL) at room temperature was added LiOH (21.64 g, 903.6 mmol). The solution was stirred for 1 h and was then concentrated under reduced pressure. The residue was adjusted to pH 6 with 1 M HCl and then extracted with DCM (3×500 mL). The combined organic layers were, dried over Na₂SO₄, filtered, and concentrated under reduced pressure to give (S)-3-(4-bromothiazol-2-yl)-2-((tert-butoxycarbonyl) amino) propanoic acid (108 g, crude). LCMS (ESI): m/z [M+H] calc'd for C₁₁H₁₆BrN₂O₄S 351.0; found 351.0.

Step 2. To a solution of(S)-3-(4-bromothiazol-2-yl)-2-((tert-butoxycarbonyl) amino) propanoic acid (70 g, 199.3 mmol) in DCM (500 mL) at 0° C. was added methyl (3S)-1,2-diazinane-3-carboxylate bis (trifluoroacetic acid) salt (111.28 g, 298.96 mmol), NMM (219.12 mL. 1993.0 mmol), EDCl (76.41 g, 398.6 mmol) and HOBt (5.39 g, 39.89 mmol). The solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel column chromatography to give methyl(S)-1-((S)-3-(4-bromothiazol-2-yl)-2-((tert-butoxycarbonyl) amino) propanoyl) hexahydropyridazine-3-carboxylate (88.1 g, 93% yield). LCMS (ESI): m/z [M+H] calc'd for C₁₇H₂₆BrN₄O₅S 477.1; found 477.1.

Step 3. To a solution of 3-(5-bromo-1-ethyl-2-(2-((S)-1-methoxyethyl) pyridin-3-yl)-1H-indol-3-yl)-2,2-dimethylpropan-1-ol (60 g, 134.7 mmol) in toluene (500 mL) at room temperature was added bis (pinacolato)diboron (51.31 g, 202.1 mmol), Pd(dppf)Cl2 (9.86 g, 13.4 mmol), and KOAc (26.44 g, 269 mmol). The reaction mixture was then heated to 90° C. and stirred for 2 h. The reaction solution was then cooled to room temperature and concentrated under reduced pressure. The residue was purified by silica gel column chromatography to give (S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (60.6 g, 94% yield). LCMS (ESI): m/z [M+H] calc'd for C₂₉H₄₂BN₂O₄ 493.32; found 493.3.

Step 4. To a solution of(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl(S) -1-((S)-3-(4-bromothiazol-2-yl)-2-((tert-butoxycarbonyl) amino) propanoyl) hexahydropyridazine-3-carboxylate (43.62 g, 91.4 mmol), K3PO4 (32.23 g, 152.3 mmol) and Pd(dppf)Cl2 (8.91 g, 12.18 mmol). The resulting solution was heated to 70° C. and stirred overnight. The reaction mixture was then cooled to room temperature and was quenched with H₂O (200 mL). The mixture was extracted with EtOAc and the combined organic layers were dried over Na₂SO₄, filtered, and concentrated under reduced pressure. The residue was purified by silica gel column chromatography to give methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate (39.7 g, 85% yield). LCMS (ESI): m/z [M+H] calc'd for C₄₀H₅₅N₆O₇S 763.4; found 763.3.

Step 5. To a solution of methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate (39.7 g, 52.0 mmol) in THF (400 mL) and H₂O (100 mL) at room temperature was added LiOH·H₂O (3.74 g, 156.2 mmol). The mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to give (S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid (37.9 g, crude). LCMS (ESI): m/z [M+H] calc'd for C₃₉H₅₃N₆O₇S 749.4; found 749.4.

Step 6. To a solution of(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCl (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel column chromatography to give tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl) carbamate (30 g, 81% yield). LCMS (ESI): m/z [M+H] calc'd for C₃₉H₅₁N₆O₆S 731.4; found 731.3.

Step 7. To a solution of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (6 g, 8.21 mmol) in DCM (60 mL) at 0° C. was added TFA (30 mL). The mixture was stirred for 1 h and was then concentrated under reduced pressure to give (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (7.0 g, crude). LCMS (ESI): m/z [M+H] calc'd for C₃₄H₄₂N₆O₄S 631.3; found: 630.3.

### Intermediate 9. Synthesis of(S)-3-bromo-5-iodo-2-(1-methoxyethyl) pyridine

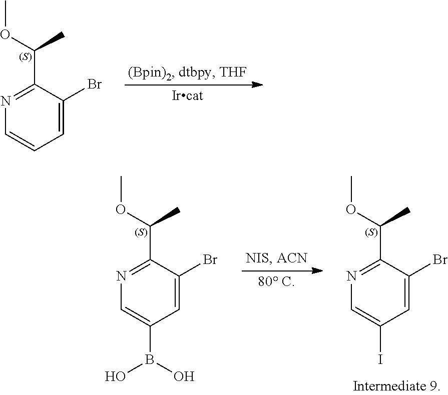

Step 1. To a stirred solution of 3-bromo-2-[(1S)-1-methoxyethyl]pyridine (80.00 g, 370.24 mmol, 1.00 equiv) and bis (pinacolato)diboron (141.03 g, 555.3 mmol, 1.50 equiv) in THF (320 mL) was added dtbpy (14.91 g, 55.5 mmol) and chloro (1,5-cyclooctadiene) iridium (I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL) and the mixture was adjusted to pH 10 with Na2CO3 (40 g) and NaOH (10 g) (mass 4:1) in water (600 mL). The aqueous layer was extracted with EtOAc (800 mL). The aqueous phase was acidified to pH=6 with HCl (6 N) to precipitate the desired solid to afford 5-bromo-6-[(1S)-1-methoxyethyl]pyridin-3-ylboronic acid (50 g, 52.0% yield) as a light-yellow solid. LCMS (ESI): m/z [M+H] calc'd for C8H11BBrNO₃ 259.0; found 260.0.

Step 2. To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na2S203 (3×500 mL). The organic layer was dried over anhydrous Na₂SO₄. After filtration, the filtrate was concentrated under reduced pressure. The residue was purified by silica gel column chromatography to afford(S)-3-bromo-5-iodo-2-(1-methoxyethyl) pyridine (20 g, 66.0% yield). LCMS (ESI): m/z [M+H] calc'd for C₈H₉BrINO 340.9; found 341.7.

### Intermediate 10. Synthesis of tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

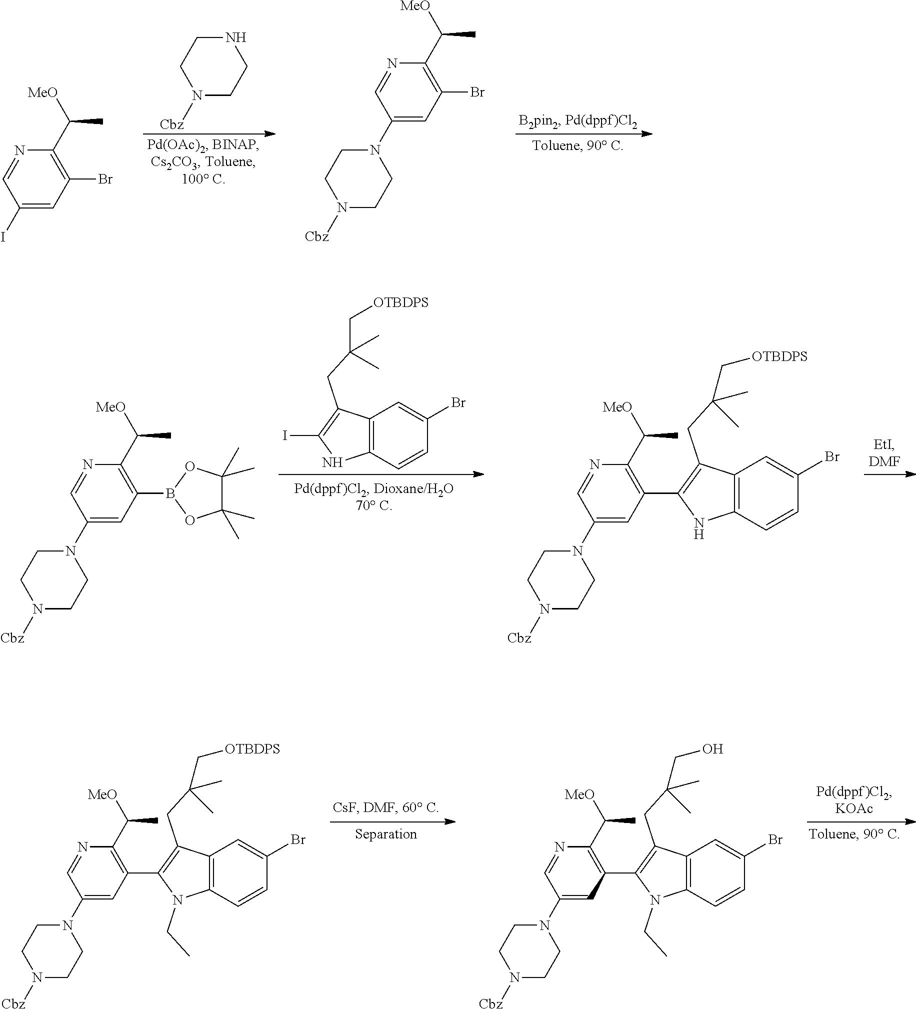

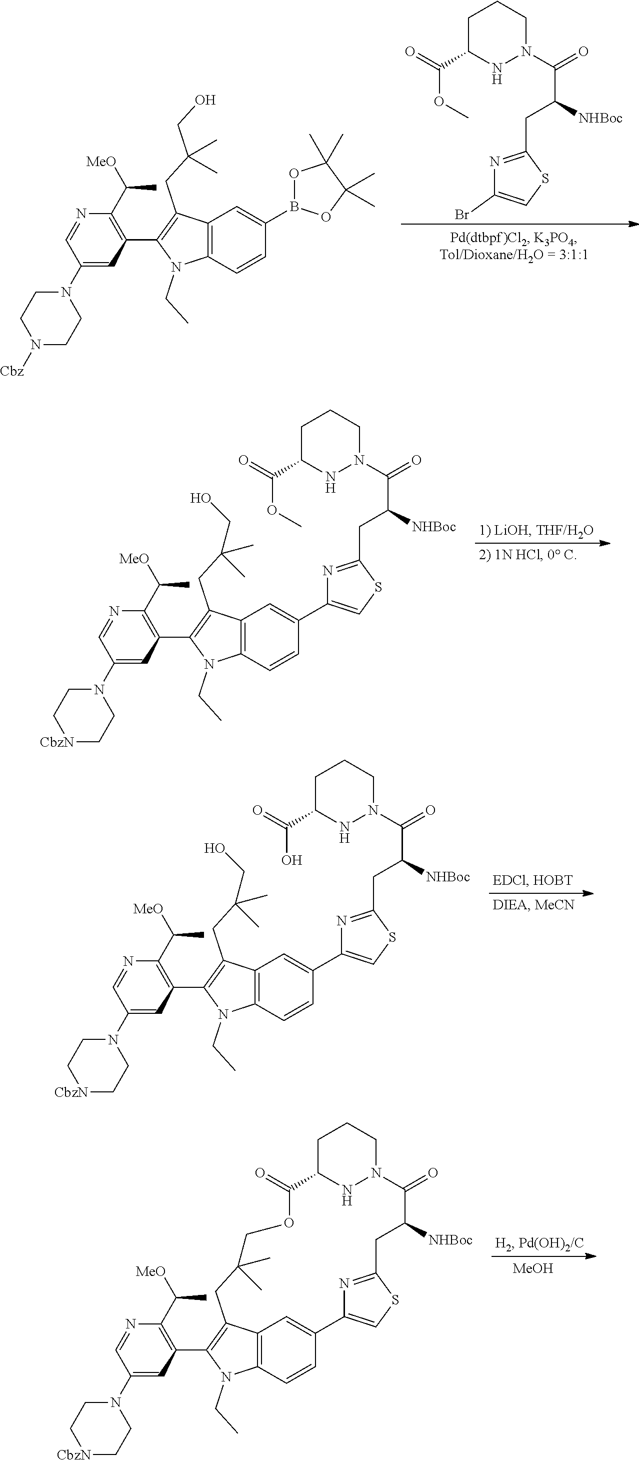

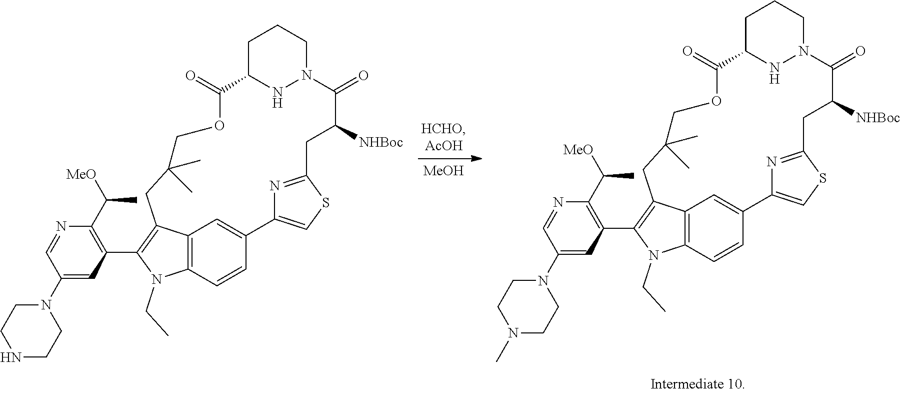

Step 1. Into a 3L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd (OAc)₂ (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was concentrated under reduced pressure. The residue was applied onto a silica gel column with ethyl acetate/hexane (1:1). Removal of solvent under reduced pressure gave benzyl(S)-4-(5-bromo-6-(1-methoxyethyl) pyridin-3-yl) piperazine-1-carboxylate (135 g, 65.1% yield) as a dark yellow solid. LCMS (ESI): m/z [M+H] calc'd for C₂₀H₂₄BrN₃O₃ 433.1; found 434.1.

Step 2. Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl(S)-4-(6-(1-methoxyethyl)-5-(4,4,5,5-tetramethyl-1,3,2-dioxaborolan-2-yl) pyridin-3-yl) piperazine-1-carboxylate (167 g, crude) as a dark yellow solid. LCMS (ESI): m/z [M+H] calc'd for C₂₆H₃₆BN₃O₅ 481.3; found 482.1.

Step 3. Into a 3-L 3-necked round-bottom flask purged and maintained with an inert atmosphere of argon, was placed(S)-4-(6-(1-methoxyethyl)-5-(4,4,5,5-tetramethyl-1,3,2-dioxaborolan-2-yl) pyridin-3-yl) piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (146 g, 48.1% yield) as a yellow solid. LCMS (ESI): m/z [M+H] calc'd for C₄₉H₅₇BrN₄O₄Si 872.3; found 873.3.

Step 4. To a stirred mixture of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (146 g, 167.0 mmol) and Cs₂CO₃ (163.28 g, 501.1 mmol) in DMF (1200 mL) was added C₂H₅I (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5L). The organic layers were dried over anhydrous Na₂SO₄. After filtration, the filtrate was concentrated under reduced pressure to give benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.

Step 5. To a stirred mixture of benzyl benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1L). Then the organic phase was concentrated under reduced pressure. The residue was purified by silica gel column chromatography, eluted with PE/EA (1/3) to afford two atropisomers of benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (38 g, 36% yield, RT=1.677 min in 3 min LCMS (0.1% FA)) and B (34 g, 34% yield, RT=1.578 min in 3 min LCMS (0.1% FA)) both as yellow solid. LCMS (ESI): m/z [M+H] calc'd for C₃₅H₄₃BrN₄O₄ 663.2; found 662.2.

Step 6. Into a 500-mL 3-necked round-bottom flask purged and maintained with an inert atmosphere of nitrogen, was placed benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (1/3) to give benzyl(S) -4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (12 g, 76.0% yield) as a yellow solid. LCMS (ESI): m/z [M+H] calc'd for C₄₁H₅₅BN₄O₆ 710.4; found 711.3.

Step 7. Into a 250-mL round-bottom flask purged and maintained with an inert atmosphere of argon, was placed benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (10.8 g, 15.2 mmol), methyl (3S)-1-[(2S)-3-(4-bromo-1,3-thiazol-2-yl)-2-[(tert-butoxycarbonyl) amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate (8 g, 50.9% yield) as a yellow solid. LCMS (ESI): m/z [M+H] calc'd for C₅₂H₆₈N₈O₉S 980.5; found 980.9.

Step 8. To a stirred mixture of methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate (12 g, 12.23 mmol) in THF (100 mL)/H₂O (100 mL) was added LiOH (2.45 g, 61.1 mmol) under N₂ atmosphere and the resulting mixture was stirred for 2 h at 25° C. Desired product could be detected by LCMS. THF was concentrated under reduced pressure. The pH of aqueous phase was acidified to 5 with HCL (1N) at 0° C. The aqueous layer was extracted with DCM (3×100 ml). The organic phase was concentrated under reduced pressure to give (S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid (10 g, 84.5% yield) as a light yellow solid. LCMS (ESI): m/z [M+H] calc'd for C₅₁H₆₆N₈O₉S 966.5; found 967.0.

Step 9. Into a 3-L round-bottom flask purged and maintained with an inert atmosphere of nitrogen, was placed(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid (18 g, 18.61 mmol), ACN (1.8 L), DIEA (96.21 g, 744.4 mmol), EDCl (107.03 g, 558.3 mmol), HOBT (25.15 g, 186.1 mmol). The resulting solution was stirred for overnight at 25° C. The resulting mixture was concentrated under reduced pressure after reaction completed. The resulting solution was diluted with DCM (1 L). The resulting mixture was washed with HCl (3×1 L, 1N aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl) amino)-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-1²-yl)-6-((S)-1-methoxyethyl) pyridin-3-yl) piperazine-1-carboxylate (10.4 g, 54.8% yield) as a light yellow solid. LCMS (ESI): m/z [M+H] calc'd for C₅₁H₆₄N₈O₈S 948.5; found 949.3.

Step 10. Into a 250-mL round-bottom flask purged and maintained with an inert atmosphere of nitrogen, was placed benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl) piperazine-1-carboxylate (10.40 g, 10.9 mmol), Pd(OH)₂/C (5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((63S,4S,Z)-11-ethyl-12-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate (8.5 g, 90.4% yield) as a light yellow solid. LCMS (ESI): m/z [M+H] calc'd for C₄₃H₅₈N₈O₆S 814.4; found 815.3.

Step 11. Into a 1000-mL round-bottom flask purged and maintained with an inert atmosphere of nitrogen, was placed tert-butyl ((6^{3S},4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 ml water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.2 g, 90.1% yield) as a yellow solid. LCMS (ESI): m/z [M+H] calc'd for C₄₄H₆₀N₈O₆S 828.4; found 829.3.

### Example A11. Synthesis of methyl (3S)-3-{[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl](methyl) carbamoyl}pyrrolidine-1-carboxylate

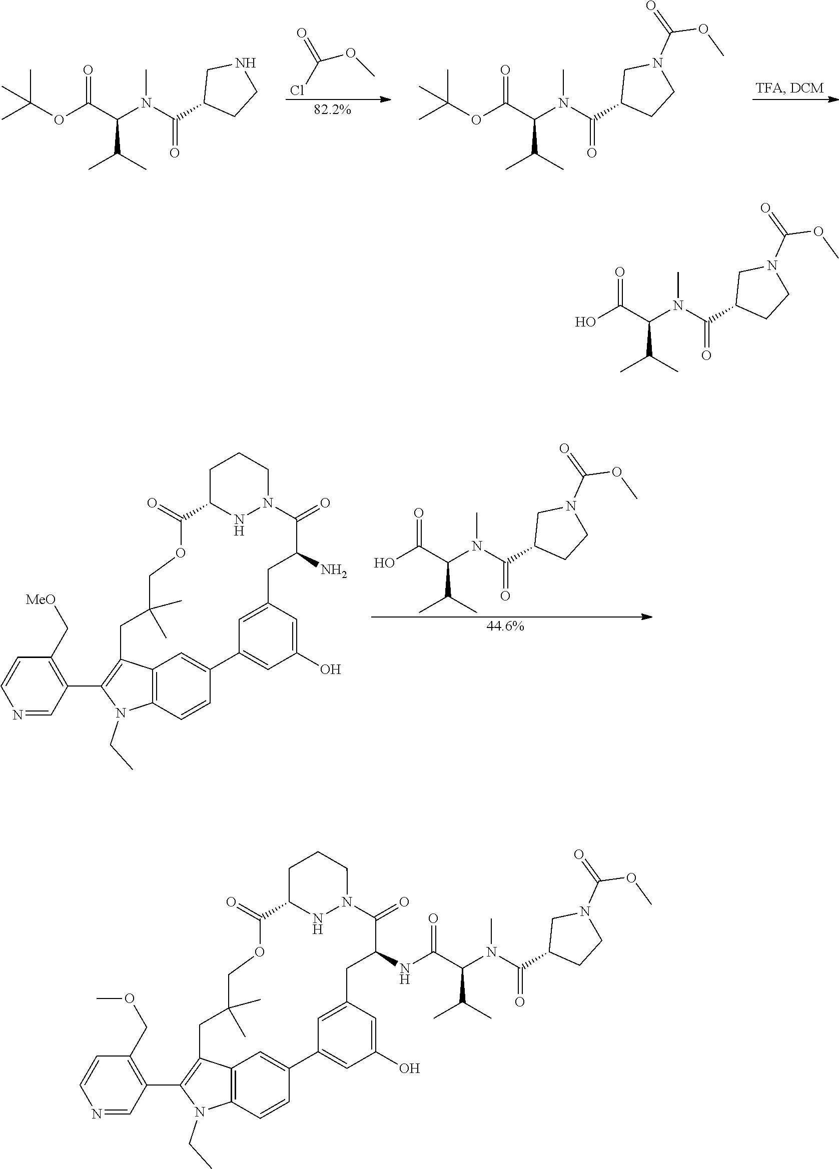

Step 1. To a mixture of tert-butyl N-methyl-N—((S)-pyrrolidine-3-carbonyl)-L-valinate (500 mg, 1.8 mmol) and TEA (356 mg, 3.5 mmol) in DCM (10 mL) at 0° C. was added methyl carbonochloridate (199 mg, 2.1 mmol) dropwise. The mixture was allowed to warm to rt and was stirred for 12 then concentrated under reduced pressure and the residue was purified by silica gel column chromatography to give methyl(S)-3-(((S)-1-(tert-butoxy)-3-methyl-1-oxobutan-2-yl) (methyl) carbamoyl) pyrrolidine-1-carboxylate (550 mg, 82%) as an oil. LCMS (ESI): m/z [M+H] calc'd for C₁₇H₃₀N₂O₅ 342.2; found 343.2.

Step 2. A mixture of methyl(S)-3-(((S)-1-(tert-butoxy)-3-methyl-1-oxobutan-2-yl) (methyl) carbamoyl) pyrrolidine-1-carboxylate (500 mg, 1.46 mmol), DCM (8 mL) and TFA (2 mL) was stirred at rt for 3 h. The mixture was concentrated under reduced pressure with azeotropic removal of H₂O using toluene (5 mL) to give N—((S)-1-(methoxycarbonyl) pyrrolidine-3-carbonyl)-N-methyl-L-valine (400 mg) as an oil. LCMS (ESI): m/z [M+H] calc'd for C₁₃H₂₂N₂O₅ 286.2; found 287.2.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (80 mg, 0.13 mmol), N—((S)-1-(methoxycarbonyl) pyrrolidine-3-carbonyl)-N-methyl-L-valine (55 mg, 0.19 mmol) and DIPEA (165 mg, 1.3 mmol) in DMF (2 mL) at 0° C. was added COMU (77 mg, 0.18 mmol). The mixture was stirred at 0° C. for 2 h, then concentrated under reduced pressure and the residue was purified by prep-HPLC to give methyl (3S)-3-{[(1S)-1-{[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl](methyl) carbamoyl}pyrrolidine-1-carboxylate (51 mg, 45% yield) as a solid. LCMS (ESI): m/z [M+H] calc'd for C₄₉H₆₃N₇O₉ 893.5; found 894.7; ¹H NMR (400 MHZ, DMSO-d₆) δ 9.33 (s, 1H), 8.88-8.66 (m, 2H), 8.62 (s, 1H), 8.17-8.06 (m, 1H), 7.92 (d, J=8.7 Hz, 1H), 7.79-7.68 (m, 1H), 7.65-7.49 (m, 2H), 7.21-7.11 (m, 1H), 7.01 (d, J=11.8 Hz, 1H), 6.71-6.40 (m, 1H), 5.54-5.30 (m, 1H), 5.28-4.99 (m, 1H), 4.87-4.56 (m, 1H), 4.46-4.21 (m, 3H), 4.11-3.89 (m, 3H), 3.70 (s, 1H), 3.65-3.59 (m, 4H), 3.35 (s, 2H), 3.24 (s, 2H), 3.18-3.07 (s, 1H), 3.00-2.58 (m, 8H), 2.22-2.01 (m, 4H), 1.81 (d, J=11.4 Hz, 2H), 1.72-1.42 (m, 2H), 1.15-0.64 (m, 13H), 0.43 (d, J=16.4 Hz, 3H).

### Example A17. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-2-{1-[(3S)-1-formylpyrrolidin-3-yl]-N-methylformamido}-3-methylbutanamide

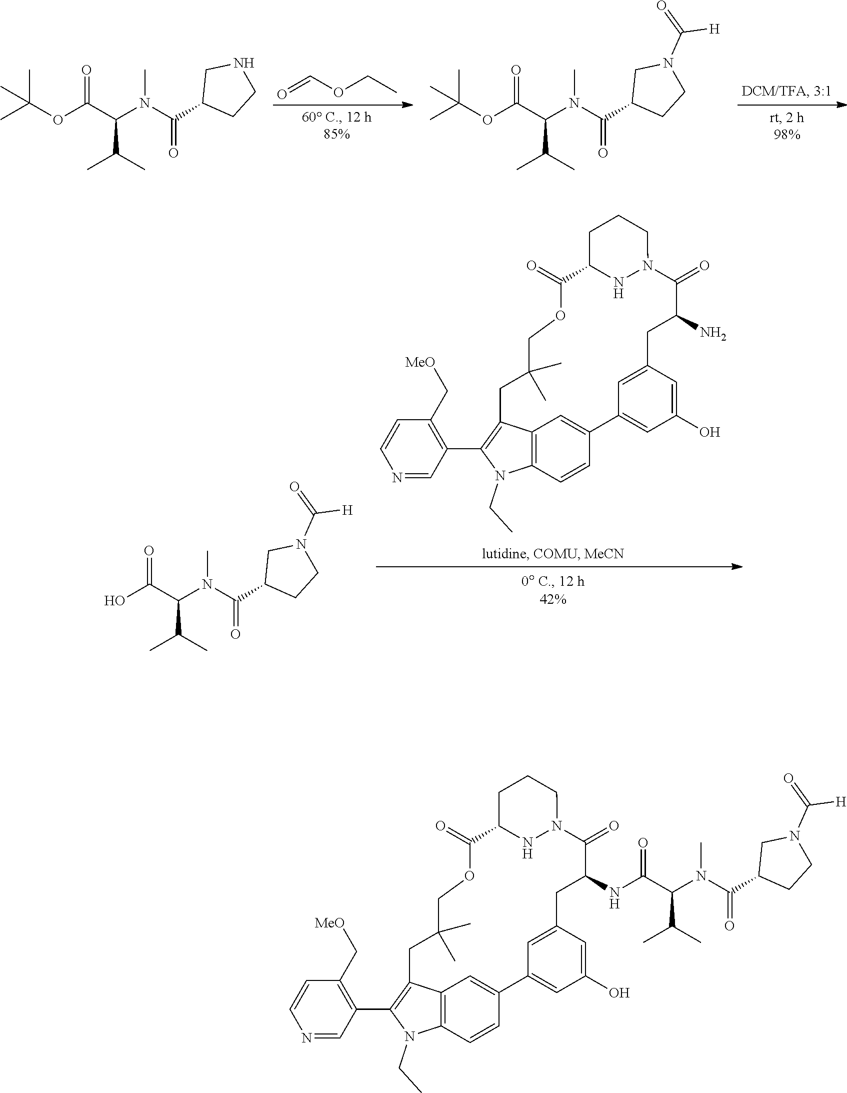

Step 1. A mixture of tert-butyl (2S)-3-methyl-2-[N-methyl-1-(3S)-pyrrolidin-3-ylformamido]butanoate (290 mg, 1.0 mmol) and ethyl formate (755 mg, 10.2 mmol) was heated to 60° C. and stirred for 12 h. The mixture was concentrated under reduced pressure to give tert-butyl (2S)-2-[1-[(3S)-1-formylpyrrolidin-3-yl]-N-methylformamido]-3-methylbutanoate (300 mg, 85% yield) as a solid. LCMS (ESI): m/z [M+H-tBu] calc'd for C₁₂H₂₀N₂O₄ 256.1; found 257.2.

Step 2. To a mixture of tert-butyl (2S)-2-[1-[(3S)-1-formylpyrrolidin-3-yl]-N-methylformamido]-3-methylbutanoate (290 mg, 0.93 mmol) in DCM (3 mL) at rt was added TFA (1 mL). The mixture was stirred at rt for 2 h, then concentrated under reduced pressure to give (2S)-2-[1-[(3S)-1-formylpyrrolidin-3-yl]-N-methylformamido]-3-methylbutanoic acid (260 mg, 98%) as an oil. LCMS (ESI): m/z [M+H] calc'd for C₁₂H₂₀N₂O₄ 256.1; found 257.2.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (60 mg, 0.1 mmol), 2,6-dimethylpyridine (15.4 mg, 0.14 mmol) and (2S)-2-[1-[(3S)-1-formylpyrrolidin-3-yl]-N-methylformamido]-3-methylbutanoic acid (37 mg, 0.14 mmol) in MeCN (2 mL) at 0° C. under an atmosphere of N₂ was added COMU (62 mg, 0.14 mmol). The mixture was stirred at 0° C. for 12 h, then concentrated under reduced pressure and the residue was purified by prep-HPLC to give (2S)—N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-{1-[(3S)-1-formylpyrrolidin-3-yl]-N-methylformamido}-3-methylbutanamide (35 mg, 42%) as a solid. LCMS (ESI): m/z [M+H] calc'd for C₄₈H₆₁N₇O₈ 863.5; found 864.5; ¹H NMR (400 MHz, DMSO-d₆) δ 8.79-8.61 (m, 2H), 8.51 (d, J=7.8 Hz, 3H), 8.31-8.09 (m, 1H), 7.93 (s, 1H), 7.68-7.48 (m, 3H), 7.25-6.97 (m, 2H), 6.71-6.43 (m, 1H), 5.40 (d, J=24.8 Hz, 1H), 5.22 (s, 1H), 4.86-4.34 (m, 1H), 4.23 (t, J=13.8 Hz, 3H), 4.12-3.84 (m, 3H), 3.83-3.54 (m, 4H), 3.22 (d, J=1.7 Hz, 2H), 3.09 (d, J=14.3 Hz, 1H), 3.01-2.92 (m, 1H), 2.99-2.93 (m, 2H), 2.92-2.65 (m, 5H), 2.07 (d, J=12.2 Hz, 4H), 1.80 (s, 1H), 1.74-1.48 (m, 2H), 1.08 (t, J=7.1 Hz, 2H), 1.03-0.54 (m, 12H), 0.43 (d, J=16.2 Hz, 3H).

### Example A6. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-{1-[(3S)-1-{2-[(3R)-3-hydroxypyrrolidin-1-yl]acetyl}pyrrolidin-3-yl]-N-methylformamido}-3-methylbutanamide

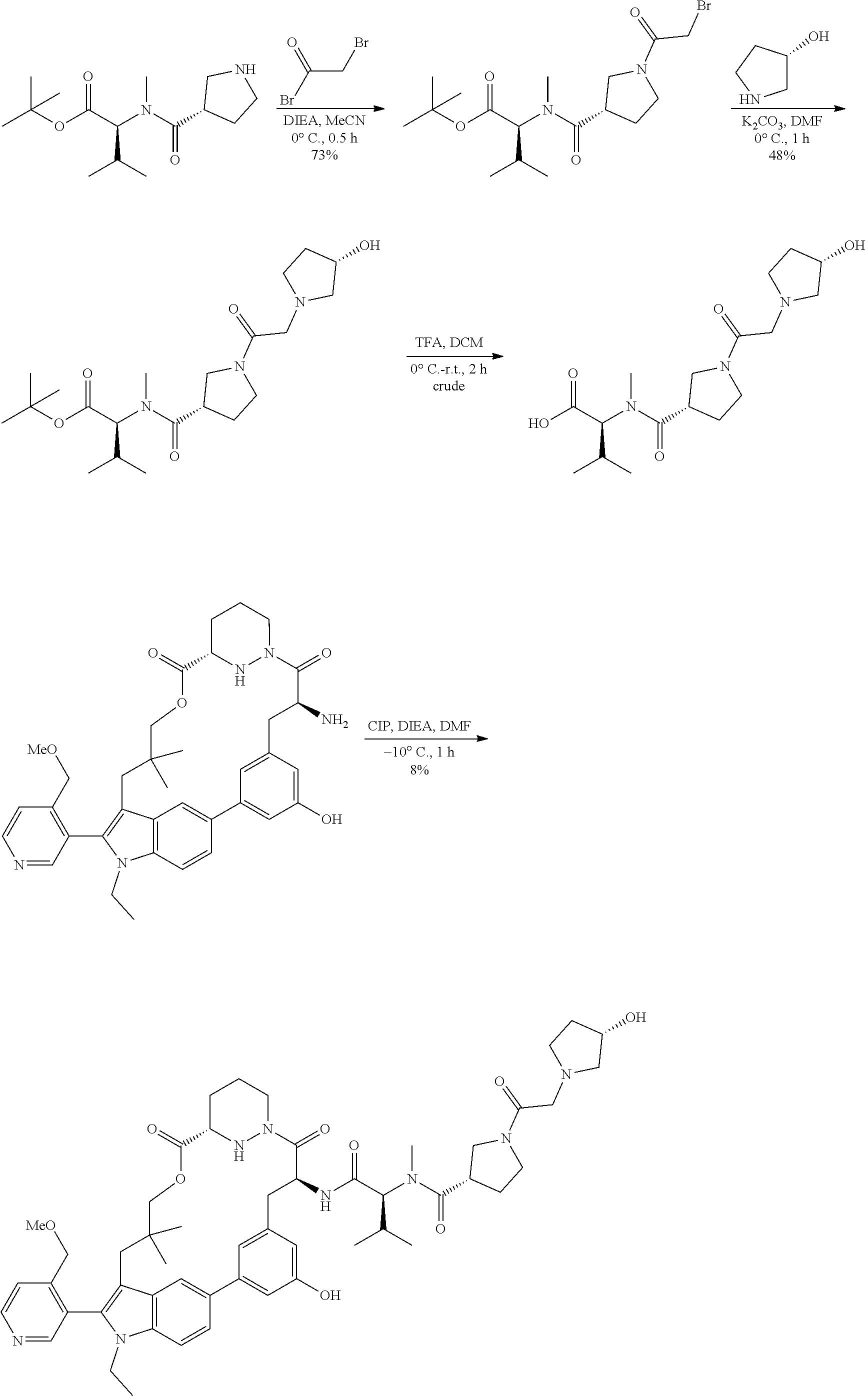

Step 1. A mixture of tert-butyl (2S)-3-methyl-2-[N-methyl-1-(3S)-pyrrolidin-3-ylformamido]butanoate (300 mg, 1.1 mmol) and DIPEA (409 mg, 3.2 mmol) in MeCN (4 mL) at 0° C. was added bromoacetyl bromide (256 mg, 1.3 mmol) dropwise. The mixture was stirred at 0° C. for 30 min, then concentrated under reduced pressure and the residue was purified by C18-silica gel column chromatography to give tert-butyl (2S)-2-[1-[(3S)-1-(2-bromoacetyl) pyrrolidin-3-yl]-N-methylformamido]-3-methylbutanoate (350 mg, 73% yield) as an oil. LCMS (ESI): m/z [M+H] calc'd for C₁₇H₂₉BrN₂O₄ 404.1; found 405.2 and 407.2.

Step 2. To a mixture of tert-butyl (2S)-2-[1-[(3S)-1-(2-bromoacetyl) pyrrolidin-3-yl]-N-methylformamido]-3-methylbutanoate (110 mg, 0.27 mmol) and K₂CO₃ (75 mg, 0.54 mmol) in DMF (2 mL) at 0° C. was added (3S)-pyrrolidin-3-ol (36 mg, 0.41 mmol) dropwise. The mixture was stirred at 0° C. for 1 h, then concentrated under reduced pressure and the residue was purified by prep-HPLC to give tert-butyl (2S)-2-[1-[(3S)-1-[2-[(3S)-3-hydroxypyrrolidin-1-yl]acetyl]pyrrolidin-3-yl]-N-methylformamido]-3-methylbutanoate (60 mg, 48% yield) as an oil. LCMS (ESI): m/z [M+H] calc'd for C₂₁H₃₇N₃O₅ 411.3; found 412.5.

Step 3. To a mixture of tert-butyl (2S)-2-[1-[(3S)-1-[2-[(3S)-3-hydroxypyrrolidin-1-yl]acetyl]pyrrolidin-3-yl]-N-methylformamido]-3-methylbutanoate (60 mg, 0.15 mmol) in DCM (0.50 mL) at 0° C. was added TFA (0.50 mL, 6.7 mmol) dropwise. The mixture was warmed to rt and stirred for 2 h, then concentrated under reduced pressure with toluene (×3) to give (2S)-2-[1-[(3S)-1-[2-[(3S)-3-hydroxypyrrolidin-1-yl]acetyl]pyrrolidin-3-yl]-N-methylformamido]-3-methylbutanoic acid (70 mg, crude) as an oil. LCMS (ESI): m/z [M+H] calc'd for C₁₇H₂₉N₃O₅ 355.2; found 356.4.

Step 4. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (60 mg, 0.1 mmol) and DIPEA (124 mg, 1.0 mmol) in DMF (1 mL) at −10° C. was added (2S)-2-[1-[(3S)-1-[2-[(3S)-3-hydroxypyrrolidin-1-yl]acetyl]pyrrolidin-3-yl]-N-methylformamido]-3-methylbutanoic acid (51 mg, 0.14 mmol) and CIP (40 mg, 0.14 mmol) in portions. The mixture was stirred at −10° C. for 1 h, then diluted with H₂O (30 mL) and extracted with EtOAc (3×10 mL). The combined organic layers were washed with brine (1×10 mL), dried over anhydrous Na₂SO₄ and filtered. The filtrate was concentrated under reduced pressure and the residue was purified by prep-HPLC to give (2S)—N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10, 14}.0^{23,27}]nonacosa-1 (26),2,4,6 (29),20,23 (27),24-heptaen-8-yl]-2-{1-[(3S)-1-{2-[(3R)-3-hydroxypyrrolidin-1-yl]acetyl}pyrrolidin-3-yl]-N-methylformamido}-3-methylbutanamide (8.6 mg, 8% yield) as a solid. LCMS (ESI): m/z [M+H] calc'd for C₅₃H₇₀N₈O₉ 962.5; found 963.5; ¹H NMR (400 MHZ, CD₃OD) δ 8.70 (td, J=5.1, 1.6 Hz, 1H), 8.66-8.48 (m, 1H), 8.07-7.90 (m, 1H), 7.76 (dd, J=9.9, 5.2 Hz, 1H), 7.61 (tt, J=9.9, 2.0 Hz, 1H), 7.52 (dt, J=8.7, 3.5 Hz, 1H), 7.11-6.97 (m, 1H), 6.62-6.47 (m, 1H), 5.68-5.48 (m, 1H), 4.79 (dt, J=11.2, 9.1 Hz, 1H), 4.53-4.18 (m, 4H), 4.16-3.86 (m, 3H), 3.85-3.56 (m, 7H), 3.55-3.46 (m, 1H), 3.42 (d, J=4.6 Hz, 4H), 3.26-3.01 (m, 3H), 3.01-2.60 (m, 9H), 2.42-2.01 (m, 6H), 1.92 (s, 1H), 1.75 (s, 2H), 1.62 (q, J=12.7 Hz, 1H), 1.26-0.80 (m, 13H), 0.61-0.40 (m, 3H).

### Example A24. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-2-{1-[(3S)-1-methanesulfonylpyrrolidin-3-yl]-N-methylformamido}-3-methylbutanamide

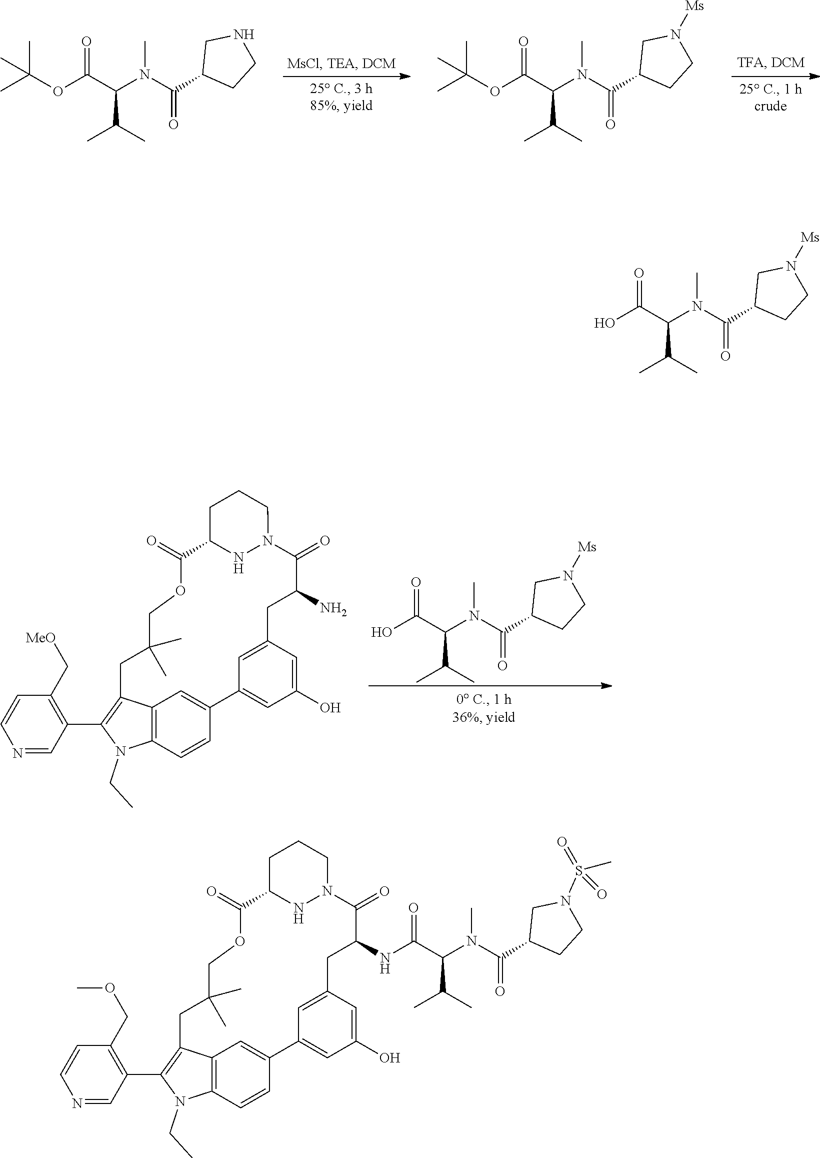 [SMILES: CC(C)[C@@H](C(=O)OC(C)(C)C)N(C)C(=O)[C@H]1CCNC1 ; CC(C)[C@@H](C(=O)OC(C)(C)C)N(C)C(=O)[C@H]1CCN(S(C)(=O)=O)C1 ; CC(C)[C@@H](C(=O)O)N(C)C(=O)[C@H]1CCN(S(C)(=O)=O)C1 ; CCn1c(-c2cnccc2COC)c2c3cc(ccc31)-c1cc(O)cc(c1)C[C@H](N)C(=O)N1CCC[C@H](N1)C(=O)OCC(C)(C)C2 ; CC(C)[C@@H](C(=O)O)N(C)C(=O)[C@H]1CCN(S(C)(=O)=O)C1 ; CCn1c(-c2cnccc2COC)c2c3cc(ccc31)-c1cc(O)cc(c1)C[C@H](NC(=O)[C@H](C(C)C)N(C)C(=O)[C@H]1CCN(S(C)(=O)=O)C1)C(=O)N1CCC[C@H](N1)C(=O)OCC(C)(C)C2]

Step 1. To a mixture of tert-butyl N-methyl-N—((S)-pyrrolidine-3-carbonyl)-L-valinate (500 mg, 1.8 mmol) in DCM (8 mL) at 0° C. under an atmosphere of N₂ was added TEA (356 mg, 3.5 mmol), followed by MsCl (242 mg, 2.1 mmol). The mixture was warmed to rt and was stirred for 3 h, then washed with brine (2×10 mL). The combined organic layers were dried over anhydrous Na₂SO₄, filtered, and the filtrate concentrated under reduced pressure and the residue was by purified by silica gel column chromatography to give tert-butyl N-methyl-N—((S)-1-(methylsulfonyl) pyrrolidine-3-carbonyl)-L-valinate (540 mg, 85%) as an oil. LCMS (ESI): m/z [M+H] calc'd for C₁₆H₃₀N₂O₅S 362.2; found 363.1.

Step 2. A mixture of tert-butyl N-methyl-N—((S)-1-(methylsulfonyl) pyrrolidine-3-carbonyl)-L-valinate (570 mg, 1.6 mmol), DCM (8 mL) and TFA (2 mL) at rt under an atmosphere of N₂ was stirred for 1 h. The mixture was concentrated under reduced pressure with toluene (5 mL) to give N-methyl-N—((S)-1-(methylsulfonyl) pyrrolidine-3-carbonyl)-L-valine (500 mg) as an oil. LCMS (ESI): m/z [M+H] calc'd for C₁₂H₂₂N₂O₅S 305.1; found 306.2.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (80 mg, 0.13 mmol) in DMF (2 mL) at 0° C. under an atmosphere of N₂ was added DIPEA (165 mg, 1.3 mmol), N-methyl-N—((S)-1-(methylsulfonyl) pyrrolidine-3-carbonyl)-L-valine (59 mg, 0.19 mmol) and COMU (71 mg, 0.17 mmol). The mixture was stirred at 0° C. for 1 h, then concentrated under reduced pressure and the residue was purified by prep-HPLC to give (2S)—N-[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-{1-[(3S)-1-methanesulfonylpyrrolidin-3-yl]-N-methylformamido}-3-methylbutanamide (42 mg, 36% yield) as a solid. LCMS (ESI): m/z [M+H] calc'd for C₄₈H₆₃N₇O₉S 913.4; found 914.6; 1H NMR (400 MHz, DMSO-d₆) δ 9.35-9.33 (m, 1H), 8.74-8.62 (m, 2H), 8.52 (s, 1H), 8.19-8.11 (m, 1H), 7.92 (s, 1H), 7.64-7.60 (m, 2H), 7.53 (t, J=9.0 Hz, 1H), 7.22-7.10 (m, 1H), 7.02 (s, 1H), 6.58-6.48 (m, 1H), 5.37-5.24 (m, 1H), 5.19-5.04 (m, 1H), 4.30-4.18 (m, 3H), 4.07-3.91 (m, 3H), 3.75-3.49 (m, 6H), 3.22 (d, J=1.5 Hz, 2H), 2.97-2.91 (m, 4H), 2.92-2.65 (m, 7H), 2.27 (s, 1H), 2.06 (d, J=14.4 Hz, 3H), 1.85 (d, J=35.3 Hz, 2H), 1.70-1.50 (m, 2H), 1.09-0.88 (m, 8H), 0.85-0.72 (m, 5H), 0.43 (d, J=17.8 Hz, 3H).

### Example A37. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-{1-[(3S)-1-[(3-hydroxyazetidin-1-yl) sulfonyl]pyrrolidin-3-yl]-N-methylformamido}-3-methylbutanamide

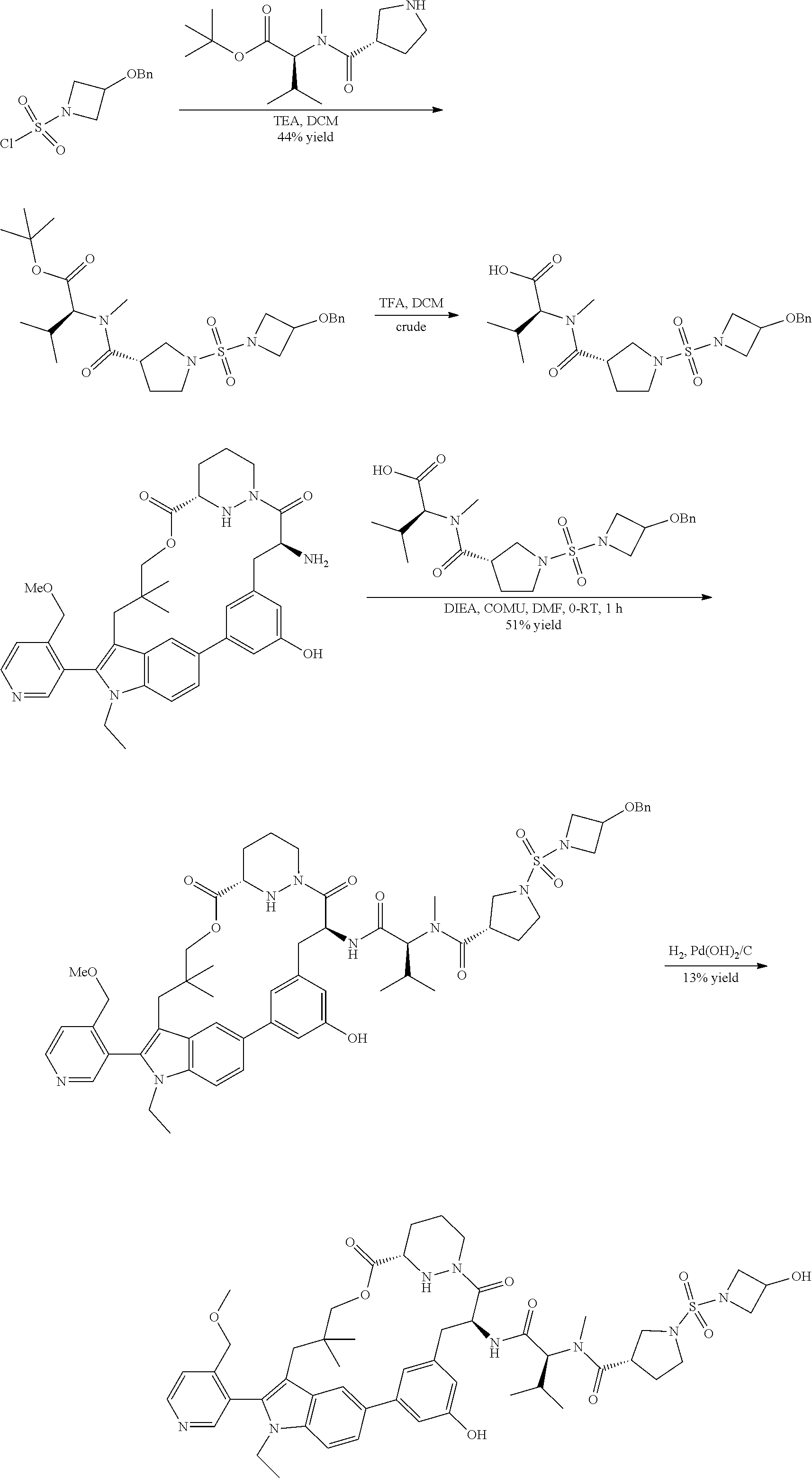

Step 1. To a mixture of tert-butyl N-methyl-N—((S)-pyrrolidine-3-carbonyl)-L-valinate (500 mg, 1.8 mmol) in DCM (20 mL) ar rt was added TEA (356 mg, 3.5 mmol) and 3-(benzyloxy) azetidine-1-sulfonyl chloride (460 mg, 1.8 mmol). The mixture was stirred at rt overnight, then concentrated under reduced pressure and the residue was purified by prep-HPLC to give tert-butyl N—((S)-1-((3-(benzyloxy) azetidin-1-yl) sulfonyl) pyrrolidine-3-carbonyl)-N-methyl-L-valinate (390 mg, 44% yield) of as an oil. LCMS (ESI): m/z [M+H] calc'd for C₂₅H₃₉N₃O₆S 509.3; found 510.5.

Step 2. A mixture of tert-butyl N—((S)-1-((3-(benzyloxy) azetidin-1-yl) sulfonyl) pyrrolidine-3-carbonyl)-N-methyl-L-valinate (390 mg, 0.77 mmol), DCM (4 mL) and TFA (1 mL) at rt under an atmosphere of N₂ was stirred at rt for 2 h. The mixture was concentrated under reduced pressure with toluene (10 mL×2) to give N—((S)-1-((3-(benzyloxy) azetidin-1-yl) sulfonyl) pyrrolidine-3-carbonyl)-N-methyl-L-valine (370 mg, crude) as a solid. LCMS (ESI): m/z [M+H] calc'd for C₂₁H₃₁N₃O₆S 453.2; found 454.5.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (60 mg, 0.1 mmol) in DMF (8 mL) at 0° C. under an atmosphere of N₂ was added DIPEA (124 mg, 0.96 mmol), N—((S)-1-((3-(benzyloxy) azetidin-1-yl) sulfonyl) pyrrolidine-3-carbonyl)-N-methyl-L-valine (65 mg, 0.14 mmol) and COMU (58 mg, 0.13 mmol). The mixture was stirred at 0° C. for 1 h, then concentrated under reduced pressure and the residue was purified by prep-HPLC to give (3S)-1-((3-(benzyloxy) azetidin-1-yl) sulfonyl)-N-((2S)-1-(((6³S,4S)-1¹-ethyl-2⁵-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide (52 mg, 51% yield) as an oil. LCMS (ESI): m/z [M+H] calc'd for C₅₇H₇₂N₈O₁₀S 1060.5; found 1061.3.

Step 4. A mixture of (3S)-1-((3-(benzyloxy) azetidin-1-yl) sulfonyl)-N-((2S)-1-(((6³S,4S)-1¹-ethyl-2⁵-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide (55 mg, 0.05 mmol), MeOH (3 mL) and Pd (OH)₂/C (11 mg, 20% by weight) was stirred under a H₂ atmosphere for 12 h. The mixture was filtered, the filtrate was concentrated under reduced pressure and the residue was purified by prep-HPLC to give (2S)—N-[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}. 1^{10, 14}.0^{23,27}]nonacosa-1 (26),2,4,6 (29),20,23 (27),24-heptaen-8-yl]-2-{1-[(3S)-1-[(3-hydroxyazetidin-1-yl) sulfonyl]pyrrolidin-3-yl]-N-methylformamido}-3-methylbutanamide (6.5 mg, 13% yield) as a solid. LCMS (ESI): m/z [M+H] calc'd for C₅₀H₆₆N₈O₁₀S 970.5; found 971.2; 1H NMR (400 MHZ, DMSO-d₆) δ 9.33-9.29 (m, 1H), 8.75-8.65 (m, 2H), 8.52 (s, 0.5H), 8.15-8.06 (m, 0.5H), 7.92 (s, 1H), 7.65-7.50 (m, 3H), 7.22-7.14 (m, 1H), 7.02 (s, 1H), 6.58-6.46 (m, 1H), 5.84-5.80 (m, 1H), 5.28-5.22 (m, 0.6H), 4.75-4.69 (m, 0.4H), 4.45-4.12 (m, 4H), 4.05-3.88 (m, 5H), 3.72-3.50 (m, 7H), 3.22 (s, 2H), 3.12-3.04 (m, 1H), 2.94-2.70 (m, 7H), 2.29-2.03 (m, 5H), 1.90-1.77 (m, 2H), 1.76-1.45 (m, 2H), 1.24 (s, 1H), 1.08-1.02 (m, 2H), 1.01-0.72 (m, 12H), 0.5-0.43 (m, 3H).

### Example A42. Synthesis of (3S)—N3-[(1S)-1-{[(8S, 14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl]-N1,N1,N3-trimethylpyrrolidine-1,3-dicarboxamide

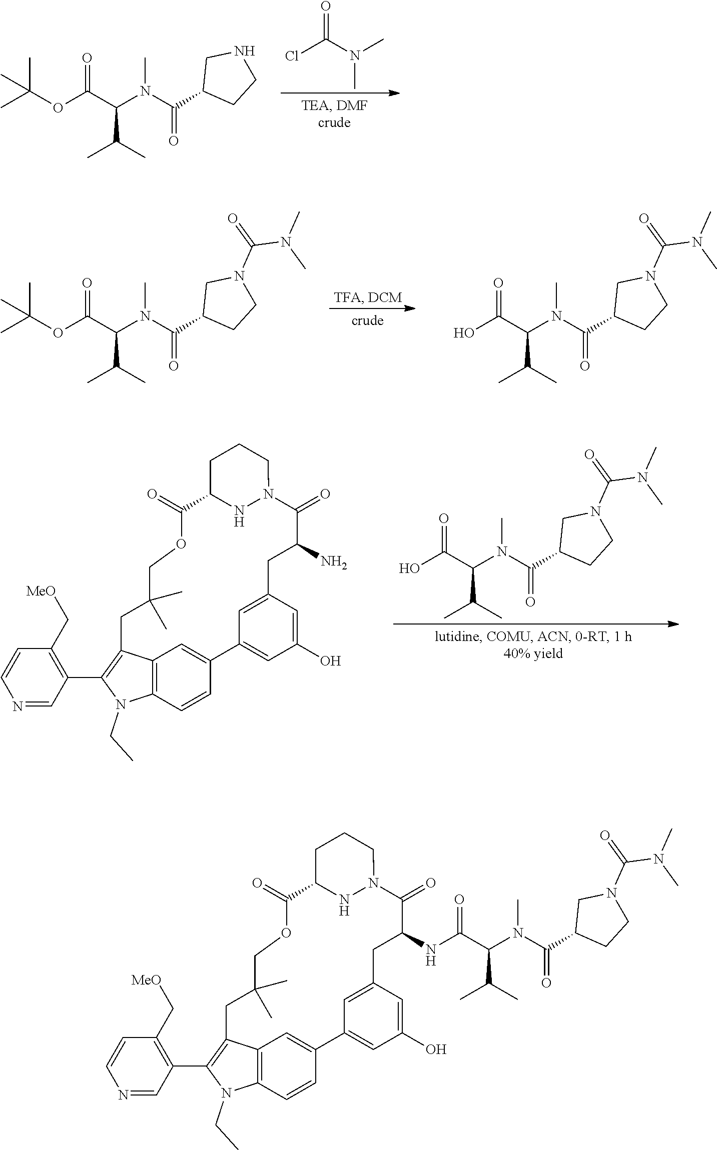

Step 1. To a mixture of tert-butyl (2S)-3-methyl-2-[N-methyl-1-(3S)-pyrrolidin-3-ylformamido]butanoate (200 mg, 0.7 mmol) and TEA (142 mg, 1.4 mmol) in DCM (10 mL) at 0° C. under an atmosphere of N₂ was added dimethylcarbamyl chloride (91 mg, 0.84 mmol) in portions. The mixture was warmed to rt and stirred for 1 h, then H₂O added and the mixture extracted with DCM (3×50 mL). The combined organic layers were washed with brine (1×5 mL), dried over anhydrous Na₂SO₄, filtered and the filtrate concentrated under reduced pressure to give tert-butyl (2S)-2-[1-[(3S)-1-(dimethylcarbamoyl) pyrrolidin-3-yl]-N-methylformamido]-3-methylbutanoate, which was used in the next step without further purification.

Step 2. A mixture of tert-butyl (2S)-2-[1-[(3S)-1-(dimethylcarbamoyl) pyrrolidin-3-yl]-N-methylformamido]-3-methylbutanoate (335 mg, 0.94 mmol) in DCM (10 mL) and TFA (2 mL, 26.9 mmol) was stirred at rt for 2 h. The mixture was concentrated under reduced pressure to give (2S)-2-[1-[(3S)-1-(dimethylcarbamoyl) pyrrolidin-3-yl]-N-methylformamido]-3-methylbutanoic acid, which was used directly in the next step without further purification. LCMS (ESI): m/z [M+H] calc'd for C₁₄H₂₅N₃O₄ 299.2; found 300.2.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (80 mg, 0.13 mmol) and (2S)-2-[1-[(3S)-1-(dimethylcarbamoyl) pyrrolidin-3-yl]-N-methylformamido]-3-methylbutanoic acid (57 mg, 0.19 mmol) in MeCN (3 mL) at 0° C. under an atmosphere of N₂ was added lutidine (137 mg, 1.3 mmol) and COMU (77 mg, 0.18 mmol) in portions. The mixture was stirred at 0° C. for 1 h, then concentrated under reduced pressure and the residue was purified by prep-HPLC to give (3S)—N3-[(1S)-1-{[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]carbamoyl}-2-methylpropyl]-N1,N1,N3-trimethylpyrrolidine-1,3-dicarboxamide (45.6 mg, 39% yield) as a solid. LCMS (ESI): m/z [M+H] calc'd for C₅₀H₆₆N₈O₈ 906.5; found 907.4; ¹H NMR (400 MHZ, DMSO-d₆) δ 9.31-9.30 (m, 1H), 8.72-8.71 (m, 1H), 8.59 (d, J=50.4 Hz, 1H), 7.92-7.90 (m, 1H), 7.74-7.42 (m, 3H), 7.23-7.08 (m, 1H), 7.00 (d, J=13.4 Hz, 1H), 6.56-6.49 (m, 1H), 5.45-5.32 (m, 1H), 5.26-5.04 (m, 1H), 4.87-4.64 (m, 1H), 4.53-4.35 (m, 1H), 4.32-4.09 (m, 3H), 4.12-3.81 (m, 3H), 3.81-3.37 (m, 6H), 3.23 (t, J=1.6 Hz, 2H), 3.12-3.10 (m, 1H), 3.01-2.52 (m, 13H), 2.23-1.95 (m, 4H), 1.81 (s, 1H), 1.67 (s, 1H), 1.60-1.47 (m, 1H), 1.28-1.22 (m, 1H), 1.21-1.14 (m, 1H), 1.11-1.02 (m, 2H), 1.02-0.66 (m, 12H), 0.43 (d, J=16.8 Hz, 3H).

### Example A27. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methyl-2-{N-methyl-1-[(3S)-1-methylpyrrolidin-3-yl]formamido}butanamide

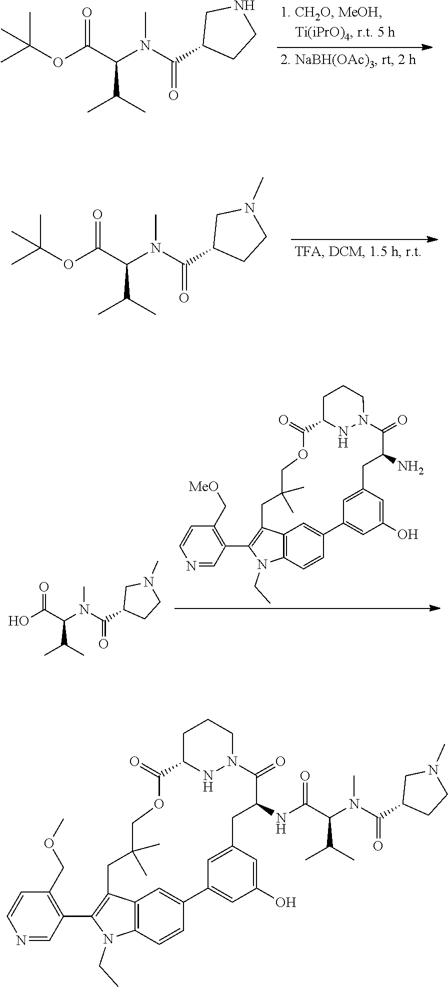

Step 1. A mixture of tert-butyl (2S)-3-methyl-2-[N-methyl-1-(3S)-pyrrolidin-3-ylformamido]butanoate (80 mg 0.28 mmol), Ti (Oi-Pr) 4 (88 mg, 0.31 mmol) and paraformaldehyde (26 mg 0.29 mmol) in MeOH (2 mL) was stirred at rt under an atmosphere of air overnight. The mixture was cooled to 0° C. and NaBH (OAc) 3 (107 mg, 0.51 mmol) was added. The mixture was warmed to rt and stirred for 2 h, then cooled to 0° C. and H₂O (0.2 mL) added. The mixture was concentrated under reduced pressure and the residue was purified by C18-silica gel column chromatography to give tert-butyl (2S)-3-methyl-2-[N-methyl-1-[(3S)-1-methylpyrrolidin-3-yl]formamido]butanoate (97 mg, crude) as an oil. LCMS (ESI): m/z [M+H] calc'd for C₁₆H₃₀N₂O₃ 298.2; found 299.3.

Step 2. A mixture of tert-butyl (2S)-3-methyl-2-[N-methyl-1-[(3S)-1-methylpyrrolidin-3-yl]formamido]butanoate (97 mg, 0.32 mmol) in DCM (2 mL) and TFA (1 mL, 13.5 mmol) was stirred at rt for 1 h, then the mixture was concentrated under reduced pressure to give (2S)-3-methyl-2-[N-methyl-1-[(3S)-1-methylpyrrolidin-3-yl]formamido]butanoic acid (100 mg, crude) as an oil. LCMS (ESI): m/z [M+H] calc'd for C₁₂H₂₂N₂O₃ 242.2; found 243.2.

Step 3. To a mixture of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (80 mg, 0.13 mmol) and (2S)-3-methyl-2-[N-methyl-1-[(3S)-1-methylpyrrolidin-3-yl]formamido]butanoic acid (47 mg, 0.19 mmol) in MeCN (2 mL) at 0° C. was added 2,6-dimethylpyridine (137 mg, 1.3 mmol) and COMU (77 mg, 0.18 mmol). The mixture was warmed to rt and stirred for 1 h, then concentrated under reduced pressure and the residue was purified by prep-HPLC to give (2S)—N-[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-3-methyl-2-{N-methyl-1-[(3S)-1-methylpyrrolidin-3-yl]formamido}butanamide (28 mg, 26% yield) as a solid. LCMS (ESI): m/z [M+H] calc'd for C₄₈H₆₃N₇O₇ 849.5; found 850.5; 1H NMR (400 MHZ, DMSO-d₆) δ 9.31 (s, 1H), 8.72 (t, J=5.1 Hz, 1H), 8.67-8.50 (m, 1H), 7.98-7.87 (m, 1H), 7.67-7.47 (m, 3H), 7.22-7.07 (m, 1H), 7.01 (s, 1H), 6.53 (d, J=40.1 Hz, 1H), 5.44-5.00 (m, 2H), 4.46-4.12 (m, 3H), 4.08-3.79 (m, 3H), 3.79-3.45 (m, 3H), 3.22 (d, J=1.2 Hz, 2H), 3.14-2.94 (m, 2H), 2.92-2.55 (m, 10H), 2.43-2.20 (m, 4H), 2.19-1.92 (m, 4H), 1.81 (d, J=11.9 Hz, 2H), 1.67 (s, 1H), 1.53 (s, 1H), 1.09 (t, J=7.1 Hz, 1H), 1.02-0.91 (m, 3H), 0.91-0.80 (m, 5H), 0.80-0.67 (m, 3H), 0.42 (d, J=21.7 Hz, 3H).

### Example A23. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29), 20,23 (27),24-heptaen-8-yl]-2-{1-[(3S)-1-(2-hydroxyethyl) pyrrolidin-3-yl]-N-methylformamido}-3-methylbutanamide

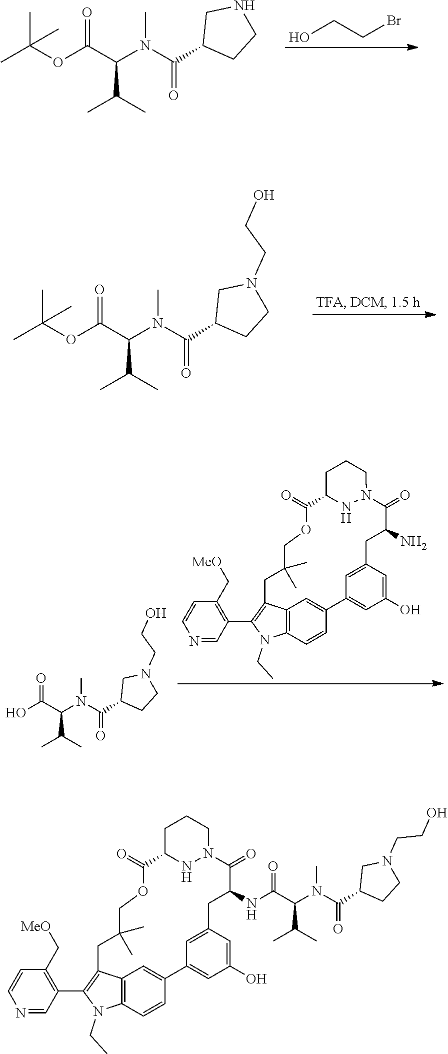

Step 1. To a mixture of tert-butyl (2S)-3-methyl-2-[N-methyl-1-(3S)-pyrrolidin-3-ylformamido]butanoate vanadium (200 mg, 0.6 mmol) and 2-bromoethanol (224 mg, 1.8 mmol) in DMF (5 mL) at rt was added Cs₂CO₃ (777 mg, 2.4 mmol) and KI (50 mg, 0.3 mmol). The mixture was stirred at rt for 16 h then diluted with H₂O and extracted with EtOAc (3×100 mL). The combined organic layers were washed with brine (2×100 mL), dried over anhydrous Na₂SO₄ and filtered. The filtrate was concentrated under reduced pressure and the residue was purified by C18-silica gel column chromatography to give tert-butyl (2S)-2-[1-[(3S)-1-(2-hydroxyethyl) pyrrolidin-3-yl]-N-methylformamido]-3-methylbutanoate (201 mg, crude) as a solid. LCMS (ESI): m/z [M+H] calc'd for C₁₇H₃₂N₂O₄ 328.2; found 329.4.

Step 2. A mixture of tert-butyl (2S)-2-[1-[(3S)-1-(2-hydroxyethyl) pyrrolidin-3-yl]-N-methylformamido]-3-methylbutanoate (100 mg, 0.3 mmol) in DCM (1 mL) and TFA (0.50 mL) at rt was stirred for 1 h, then concentrated under reduced pressure to give (2S)-2-[1-[(3S)-1-(2-hydroxyethyl) pyrrolidin-3-yl]-N-methylformamido]-3-methylbutanoic acid (110 mg, crude) as an oil. LCMS (ESI): m/z [M+H] calc'd for C₁₃H₂₄N₂O₄ 272.2; found 273.2.

Step 3. To a mixture of (6³S,4S)-4-amino-1¹-ethyl-2⁵-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (60 mg, 0.1 mmol) and (2S)-2-[1-[(3S)-1-(2-hydroxyethyl) pyrrolidin-3-yl]-N-methylformamido]-3-methylbutanoic acid (31 mg, 0.11 mmol) in MeCN (2 mL) at 0° C. under an atmosphere of N₂ was added 2,6-dimethylpyridine (103 mg, 1.0 mmol) and COMU (58 mg, 0.13 mmol). The mixture was warmed to rt and stirred for 1 h, then concentrated under reduced pressure and the residue was purified by prep-HPLC to give (2S)—N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-{1-[(3S)-1-(2-hydroxyethyl) pyrrolidin-3-yl]-N-methylformamido}-3-methylbutanamide (13 mg, 16% yield) as a solid. LCMS (ESI): m/z [M+H] calc'd for C₄₉H₆₅N₇O₈ 879.5; found 880.3; ¹H NMR (400 MHZ, DMSO-d₆) δ 8.72 (t, J=5.3 Hz, 1H), 8.68-8.58 (m, 1H), 8.52 (s, 1H), 7.93 (d, J=10.6 Hz, 1H), 7.68-7.58 (m, 2H), 7.53 (d, J=7.1 Hz, 1H), 7.21-7.07 (m, 1H), 7.01 (s, 1H), 6.52 (d, J=42.8 Hz, 1H), 5.35 (d, J=25.5 Hz, 1H), 5.22-4.97 (m, 1H), 4.59-4.35 (m, 1H), 4.23 (t, J=13.8 Hz, 3H), 4.11-3.81 (m, 3H), 3.81-3.56 (m, 2H), 3.56-3.47 (m, 3H), 3.22 (d, J=1.2 Hz, 2H), 3.09 (d, J=12.6 Hz, 1H), 2.99-2.65 (m, 10H), 2.57-2.53 (m, 1H), 2.47-2.19 (m, 2H), 2.14-2.08 (m, 1H), 2.08 (s, 1H), 2.06-1.98 (m, 2H), 1.81 (s, 2H), 1.59 (d, J=55.9 Hz, 2H), 1.14-0.67 (m, 13H), 0.42 (d, J=22.1 Hz, 3H).

### Example A57. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methyl-2-(N-methylmethanesulfonamido) butanamide

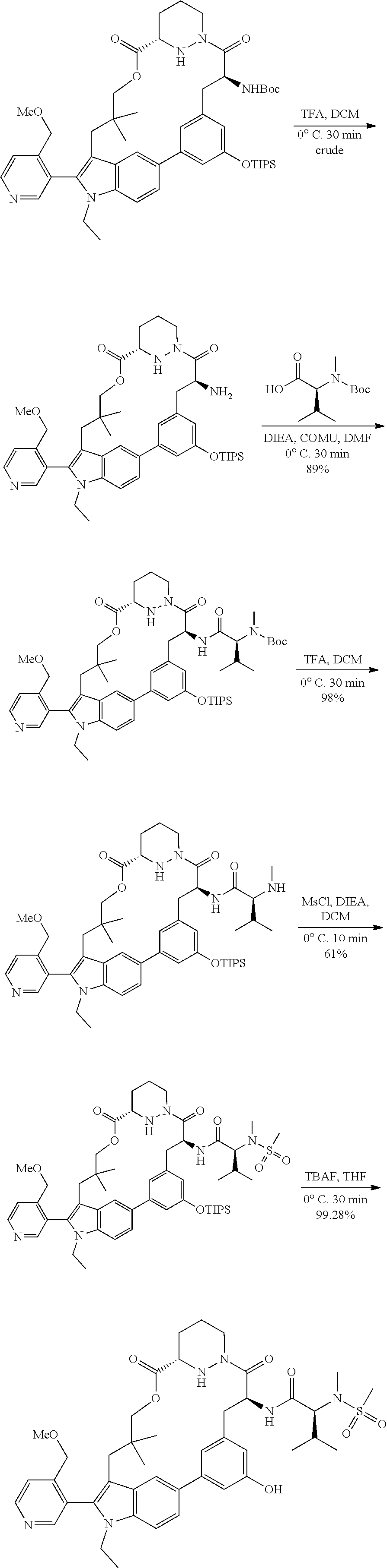

Step 1. A mixture of tert-butyl N-[(8S,14S)-22-ethyl-21-[2-(2-methoxyethyl)phenyl]-18,18-dimethyl-9, 15-dioxo-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (880 mg, 1.2 mmol), DCM (10 mL) and TFA (5 mL) was stirred at 0° C. for 30 min. The mixture was concentrated under reduced pressure to give (8S, 14S)-8-amino-22-ethyl-21-[4-(methoxymethyl)pyridin-3-yl]-18, 18-dimethyl-4-[(triisopropylsilyl)oxy]-16-oxa-10,22,28-triazapentacyclo[18.5.2.1\hat{ }[2,6]0.1\hat{ }[10,14]0.0\hat{ }[23,27]nonacosa-1 (26),2,4,6 (29),20,23 (27),24-heptaene-9, 15-dione, that was used directly in the next step without further purification. LCMS (ESI): m/z [M+H] calc'd for C₄₅H₆₃N₅O₅Si 781.5; found 782.7.

Step 2. To a mixture of (8S, 14S)-8-amino-22-ethyl-21-[4-(methoxymethyl)pyridin-3-yl]-18,18-dimethyl-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaene-9,15-dione (880 mg, 1.13 mmol) and (2S)-2-[(tert-butoxycarbonyl) (methyl) amino]-3-methylbutanoic acid (521 mg, 2.3 mmol) in DMF (8.8 mL) at 0° C. was added DIPEA (1.45 g, 11.3 mmol) and COMU (88 mg, 0.21 mmol). The mixture was stirred at 0° C. for 30 min, then diluted with H₂O (100 mL) and extracted with EtOAc (3×100 mL). The combined organic layers were washed with brine (3×100 mL), dried over anhydrous Na₂SO₄ and filtered. The filtrate was concentrated under reduced pressure and the residue was purified by prep-TLC to give tert-butyl N-[(1S)-1-[[(8S, 14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]]nonacosa-1 (26),2,4,6 (29),20,23 (27),24-heptaen-8-yl]carbamoyl]-2-methylpropyl]-N-methylcarbamate (1 g, 89% yield) as a solid. LCMS (ESI): m/z [M+H] calc'd for C₅₆H₈₂N₆O₈Si 994.6; found 995.5.

Step 3. A mixture of tert-butyl N-[(1S)-1-[[(8S,14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]. 1\hat{ }[10, 14]0.0\hat{ }[23,27]nonacosa-1 (26),2,4,6 (29),20,23 (27),24-heptaen-8-yl]carbamoyl]-2-methylpropyl]-N-methylcarbamate (1.0 g, 1.0 mmol), DCM (10 mL) and TFA (5 mL) was stirred for 30 min. The mixture was concentrated under reduced pressure and the residue was basified to PH ˜8 with saturated NaHCO₃, then extracted with EtOAc (3×10 mL). The combined organic layers were washed with brine (3×10 mL), dried over anhydrous Na₂SO₄, filtered and the filtrate concentrated under reduced pressure to give (2S)—N-[(8S, 14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]-3-methyl-2-(methylamino) butanamide (880 mg, 98% yield) as a solid. LCMS (ESI): m/z [M+H] calc'd for C₅₁H₇₄N₆O₆Si 894.5; found 895.5.

Step 4. To a mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]-3-methyl-2-(methylamino) butanamide (90 mg, 0.1 mmol) in DCM (2 mL) at 0° C. was added DIPEA (65 mg, 0.5 mmol) and MsCl (14 mg, 0.12 mmol). The mixture was stirred at 0 C for 30 min, then concentrated under reduced pressure and the residue diluted with H₂O (5 mL) and extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26), 2,4,6 (29),20,23 (27),24-heptaen-8-yl]-3-methyl-2-(N-methylmethanesulfonamido) butanamide (60 mg, 61% yield) as a solid. LCMS (ESI): m/z [M+H] calc'd for C₅₂H₇₆N₆O₈SSi 972.5; found 973.7.

Step 5. To a mixture of (2S)—N-[(8S,14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6]. 1\hat{ }[10, 14]0.0\hat{ }[23,27]nonacosa-1 (26), 2,4,6 (29),20,23 (27),24-heptaen-8-yl]-3-methyl-2-(N-methylmethanesulfonamido) butanamide (60 mg, 0.06 mmol) in THF (2 mL) at 0° C. was added 1M TBAF in THF (6 □L, 0.006 mmol). The mixture was stirred at 0° C. for 30 min, then diluted with H₂O (5 mL) and extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methyl-2-(N-methylmethanesulfonamido) butanamide (50 mg, 99% yield) as a solid. LCMS (ESI): m/z [M+H] calc'd for C₄₃H₅₆N₆O₈S 816.4; found 817.5; 1H NMR (400 MHZ, DMSO-d₆) δ 9.34 (d, J=1.8 Hz, 1H), 8.72 (t, J=5.2 Hz, 1H), 8.65 (d, J=5.8 Hz, 1H), 7.99-7.86 (m, 1H), 7.71-7.45 (m, 3H), 7.19 (d, J=41.5 Hz, 1H), 7.03 (t, J=1.9 Hz, 1H), 6.66 (d, J=10.4 Hz, 1H), 5.34 (q, J=8.1 Hz, 1H), 5.14 (dd, J=62.7, 12.2 Hz, 1H), 4.55-4.15 (m, 3H), 4.14-3.80 (m, 4H), 3.80-3.46 (m, 3H), 3.23 (s, 1H), 3.02-2.72 (m, 8H), 2.68 (s, 2H), 2.15-1.89 (m, 3H), 1.82 (d, J=12.4 Hz, 1H), 1.76-1.62 (m, 1H), 1.54 (q, J=12.7 Hz, 1H), 1.24 (s, 1H), 1.08 (t, J=7.1 Hz, 2H), 1.03-0.86 (m, 9H), 0.81 (s, 2H), 0.46 (s, 3H).

### Example A43. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-2-(2-hydroxy-N-methylacetamido)-3-methylbutanamide

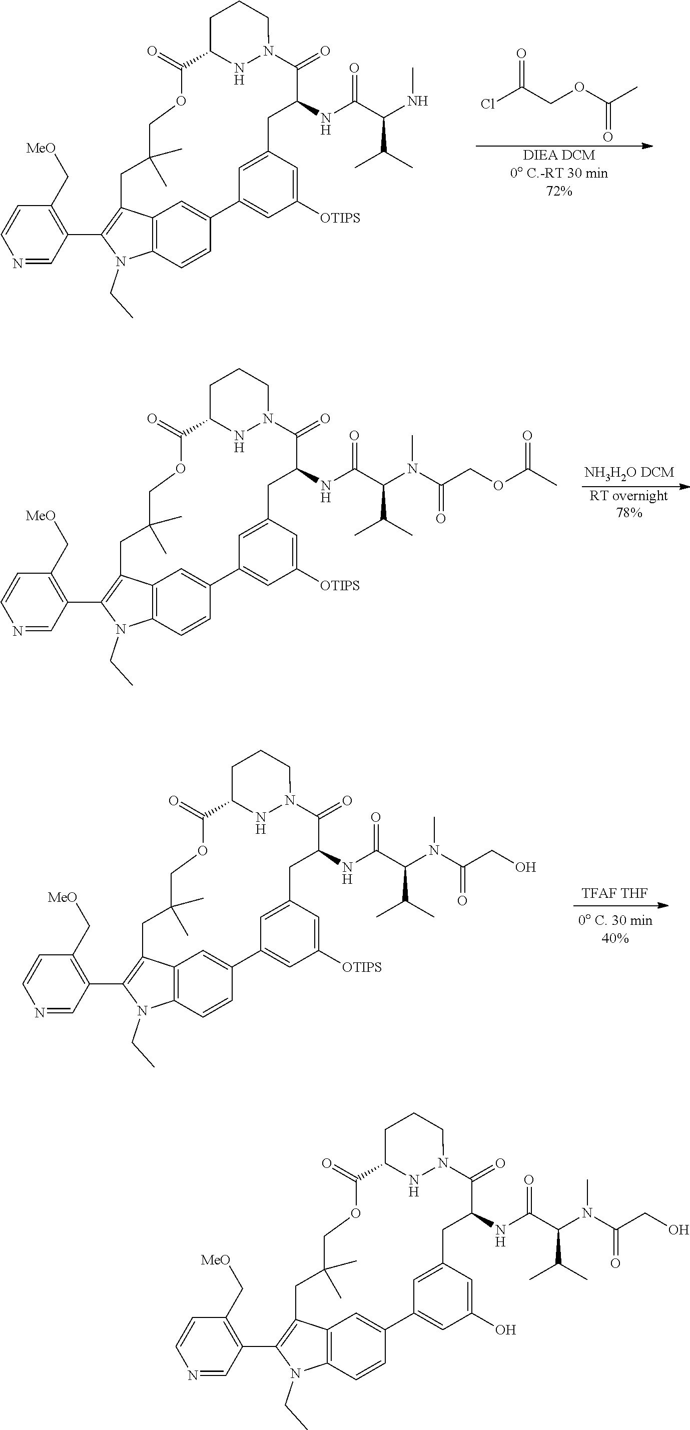

Step 1. To a mixture of (2S)—N-[(8S, 14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]. 1\hat{ }[10, 14]0.0\hat{ }[23,27]nonacosa-1 (26),2,4,6 (29),20,23 (27),24-heptaen-8-yl]-3-methyl-2-(methylamino) butanamide (100 mg, 0.11 mmol) in DCM (1 mL) at 0° C. was added DIPEA (72 mg, 0.56 mmol) and 2-chloro-2-oxoethyl acetate (11.53 mg, 0.11 mmol). The mixture was warmed to rt and stirred for 30 min, then concentrated under reduced pressure, diluted with water (3 mL) and extracted with EtOAc (3×3 mL). The combined organic layers were washed with brine (3×3 mL), dried over anhydrous Na₂SO₄ and filtered. The filtrate was concentrated under reduced pressure and the residue was purified by prep-TLC to give [[(1S)-1-[(8S,14S)-22-ethyl-21-[4-(methoxymethyl)pyridin-3-yl]-18, 18-dimethyl-9,15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]]nonacosa-1 (26), 2,4,6 (29),20,23 (27),24-heptaen-8-yl]carbamoyl]-2-methylpropyl](methyl) carbamoyl]methyl acetate (80 mg, 72% yield) as a solid. LCMS (ESI): m/z [M+H] calc'd for C₅₅H₇₈N₆OgSi 994.6; found 995.7.

Step 2. A mixture of [[(1S)-1-[(8S,14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26), 2,4,6 (29),20,23 (27),24-heptaen-8-yl]carbamoyl]-2-methylpropyl](methyl) carbamoyl]methyl acetate (80 mg, 0.080 mmol), DCM (1 mL) and aqueous NH₄OH (0.8 mL) was stirred at rt overnight. H₂O (5 mL) was added and the mixture was extracted with EtOAc (3×5 mL). The combined organic layers were washed with brine (3×5 mL), dried over anhydrous Na₂SO₄ and filtered. The filtrate was concentrated under reduced pressure and the residue was purified by prep-TLC to give (2S)—N-[(8S,14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26), 2,4,6 (29),20,23 (27),24-heptaen-8-yl]-2-(2-hydroxy-N-methylacetamido)-3-methylbutanamide (60 mg, 78% yield) as a solid. LCMS (ESI): m/z [M+H] calc'd for C₅₃H₇₆N₆O₈Si 952.6; found 953.7.

Step 3. A mixture of (2S)—N- [(8S,14S)-22-ethyl-21-[4-(methoxymethyl) pyridin-3-yl]-18,18-dimethyl-9, 15-dioxo-4-[(triisopropylsilyl)oxy]-16-oxa-10,22,28-triazapentacyclo[18.5.2.1\hat{ }[2,6]. 1\hat{ }[10, 14]0.0\hat{ }[23,27]nonacosa-1 (26), 2,4,6 (29),20,23 (27),24-heptaen-8-yl]-2-(2-hydroxy-N-methylacetamido)-3-methylbutanamide (60 mg, 0.06 mmol), THF (2 mL) and 1M TBAF in THF (6 DL, 0.006 mmol) at 0° C. was stirred for 30 min. H₂O (3 mL) was added and the mixture was extracted with EtOAc (3×3 mL). The combined organic layers were washed with brine (3×3 mL), dried over anhydrous Na₂SO₄. The filtrate was concentrated under reduced pressure and the residue was purified by prep-TLC to give (2S)—N-[(8S, 14S)-22-ethyl-4-hydroxy-21-[4-(methoxymethyl)pyridin-3-yl]-18, 18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-2-(2-hydroxy-N-methylacetamido)-3-methylbutanamide (20 mg, 40% yield) as a solid. LCMS (ESI): m/z [M+H] calc'd for C₄₄H₅₆N₆O₈ 796.4; found 797.6; 1H NMR (400 MHZ, CD₃OD) δ 8.70 (dd, J=5.7, 4.4 Hz, 1H), 8.66-8.49 (m, 1H), 8.00 (dd, J=4.6, 1.7 Hz, 1H), 7.76 (dd, J=9.9, 5.2 Hz, 1H), 7.60 (dt, J=8.7, 1.6 Hz, 1H), 7.56-7.47 (m, 1H), 7.29-7.18 (m, 1H), 7.10-6.98 (m, 1H), 6.54 (dt, J=3.6, 1.7 Hz, 1H), 5.67-5.55 (m, 1H), 4.77 (dd, J=11.2, 8.4 Hz, 1H), 4.57-4.39 (m, 3H), 4.39-4.20 (m, 3H), 4.19-3.91 (m, 2H), 3.90-3.65 (m, 3H), 3.60 (dd, J=11.0, 1.8 Hz, 1H), 3.42 (s, 1H), 3.32 (s, 1H), 3.29-3.15 (m, 1H), 3.10-2.97 (m, 1H), 2.97-2.82 (m, 5H), 2.82-2.63 (m, 2H), 2.35-2.11 (m, 3H), 1.94 (d, J=13.2 Hz, 1H), 1.82-1.49 (m, 3H), 1.31 (s, 1H), 1.19 (t, J=7.2 Hz, 2H), 1.09-0.95 (m, 7H), 0.95-0.83 (m, 5H), 0.50 (d, J=32.4 Hz, 3H).

### Example A50. Synthesis of oxolan-3-yl-N-[(1S)-1-{[(8S,14S)-22-ethyl-4-hydroxy-21-[4-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl]-N-methylcarbamate

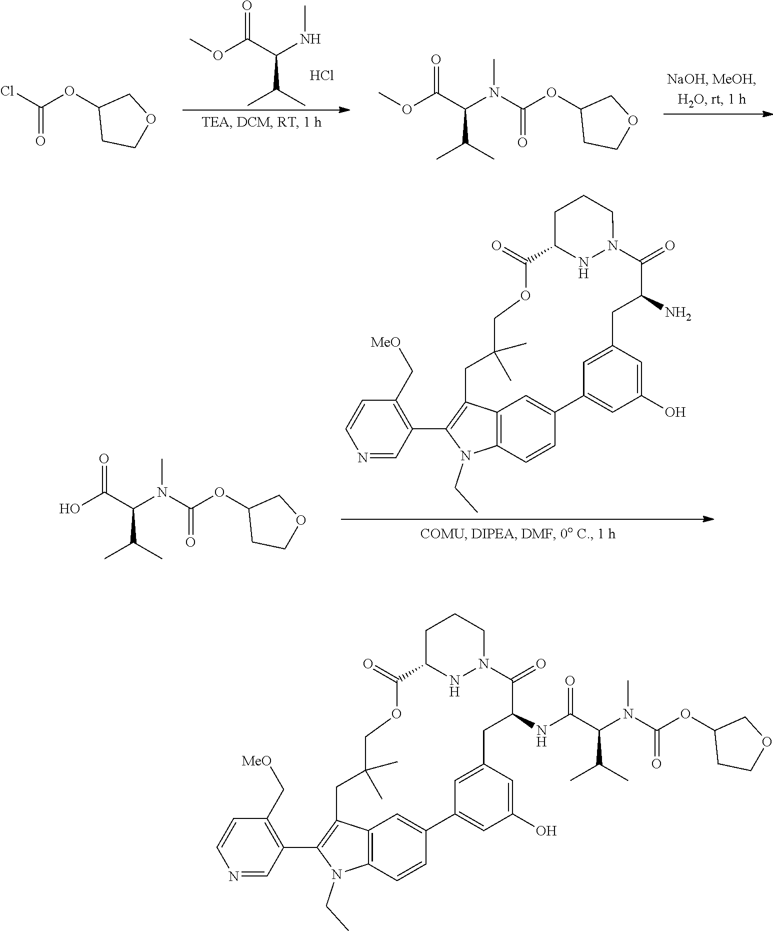

Step 1. To a mixture of methyl (2S)-3-methyl-2-(methylamino) butanoate (500 mg, 3.4 mmol) and TEA (1.44 mL, 14.2 mmol) in DCM (20 mL) at rt was added oxolan-3-yl carbonochloridate (1.04 g, 6.9 mmol). The mixture was stirred at rt for 1 h, then sat. NH₄Cl added and the mixture extracted with DCM (3×10 mL). The combined organic layers were washed with brine (1×10 mL), dried over anhydrous Na₂SO₄ and filtered. The filtrate was concentrated under reduced pressure and the residue was purified by silica gel column chromatography to give methyl (2S)-3-methyl-2 [methyl (oxolan-3-yloxy) carbonyl]amino]butanoate (800 mg, 89% yield) as an oil. 1H NMR (300 MHZ, CDCl₃) δ 4.57-4.05 (m, 1H), 3.99-3.78 (m, 4H), 3.70 (s, 3H), 3.26 (s, 1H), 2.99-2.68 (m, 3H), 2.26-1.83 (m, 3H), 1.06-0.76 (m, 6H).

Step 2. A mixture of methyl (2S)-3-methyl-2 [methyl (oxolan-3-yloxy) carbonyl]amino]butanoate (1 g, 3.9 mmol) and 2M NaOH (19.3 mL, 38.6 mmol) in MeOH (20 mL) was stirred at rt for 1 h. The mixture was concentrated under reduced pressure and the residue was extracted with MTBE (3×10 mL). The aqueous layer was acidified to PH ˜2 with 2 M HCl then extracted with DCM (3×20 mL). The combined organic layers were washed with brine (2×10 mL), dried over anhydrous Na₂SO₄, filtered and the filtrate was concentrated under reduced pressure to give (2S)-3-methyl-2-[methyl [(oxolan-3-yloxy) carbonyl]amino]butanoic acid (630 mg, 67% yield) as an oil. 1H NMR (300 MHZ, CDCl₃) δ 5.32 (br. s, 1H), 4.45-4.08 (m, 1H), 4.04-3.81 (m, 4H), 2.93 (d, J=6.9 Hz, 3H), 2.38-1.93 (m, 3H), 1.06 (t, J=5.6 Hz, 3H), 0.94 (d, J=6.7 Hz, 3H).

Step 3. To a mixture of (6³S,4S)-4-amino-1¹-ethyl-2⁵-hydroxyl-1²-(4-(methoxymethyl)pyridin-3-yl)-10, 10-dimethyl-6¹,6²,6³,6⁴,6⁵,6⁶-hexahydro-11H-8-oxa-1 (5,3)-indola-6 (1,3)-pyridazina-2 (1,3)-benzenacycloundecaphane-5,7-dione (80 mg, 0.13 mmol), (2S)-3-methyl-2-[methyl [(oxolan-3-yloxy) carbonyl]amino]butanoic acid (63 mg, 0.26 mmol) and DIPEA (165 mg, 1.3 mmol) in DMF (2 mL) at 0° C. was added COMU (38 mg, 0.19 mmol). The mixture was stirred at 0° C. for 30 min, then the mixture was concentrated under reduced pressure and the residue was purified by prep-HPLC to give oxolan-3-yl-N-[(1S)-1-{[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]carbamoyl}-2-methylpropyl]-N-methylcarbamate (50 mg, 45% yield) as a solid. LCMS (ESI): m/z [M+H] calc'd for C₄₇H₆₀N₆O₉ 852.4; found 853.5; 1H NMR (400 MHZ, DMSO-d₆) δ 9.34-9.18 (m, 1H), 8.72 (t, J=5.1 Hz, 1H), 8.58 (d, J=47.8 Hz, 1H), 8.48-8.15 (m, 1H), 7.91 (s, 1H), 7.70-7.57 (m, 2H), 7.55-7.46 (m, 1H), 7.13 (d, J=24.7 Hz, 1H), 7.01 (s, 1H), 6.56 (d, J=9.2 Hz, 1H), 5.34 (s, 1H), 5.28-5.00 (m, 2H), 4.40 (d, J=13.3 Hz, 1H), 4.33-4.14 (m, 4H), 4.12-3.45 (m, 10H), 3.23 (s, 1H), 3.10 (d, J=14.5 Hz, 1H), 2.99-2.62 (m, 6H), 2.20-1.99 (m, 4H), 1.80 (s, 1H), 1.66 (s, 1H), 1.52 (d, J=12.2 Hz, 1H), 1.09 (t, J=7.1 Hz, 2H), 0.99-0.89 (m, 6H), 0.87-0.76 (m, 5H), 0.42 (d, J=24.2 Hz, 3H).

### Example A277. The synthesis of (2S)—N-((6³S,4S,Z)-1¹-ethyl-1²-(2-((S)-1-methoxyethyl)-5-(4-methylpiperazin-1-yl) pyridin-3-yl)-10,10-trimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-3-methyl-2-(1,3,3-trimethylureido) butanamide

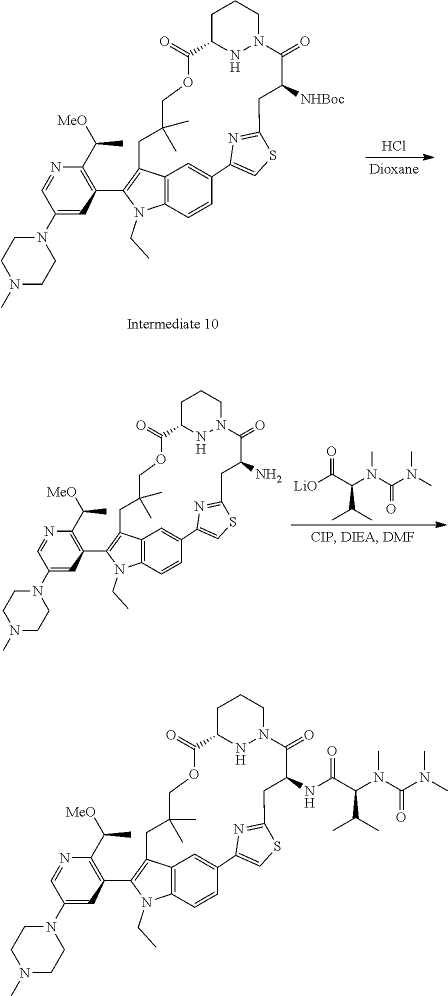

Step 1. A solution of Intermediate 10 (8.2 g, 9.89 mmol) in dioxane (40 mL) at 0° C. under nitrogen atmosphere, was added HCl (40 mL, 4M in dioxane). The reaction solution was stirred at 0° C. for 1 h, then concentrated under reduced pressure. The resulting mixture was diluted with DCM (600 mL) and saturated sodium bicarbonate aqueous solution (400 mL). The organic phase was separated and washed with brine (500 mL×2), then concentrated under reduced pressure to afford (63S,4S,Z)-4-amino-11-ethyl-12-(2-((S)-1-methoxyethyl)-5-(4-methylpiperazin-1-yl) pyridin-3-yl)-10,10-dimethyl-6¹,6²,6³,6⁴,6⁵,6⁶-hexahydro-11H-8-oxa-2 (4,2)-thiazola-1 (5,3)-indola-6 (1,3)-pyridazinacycloundecaphane-5,7-dione (7.2 g, 94.8% yield) as solid. LCMS (ESI): m/z [M+H] calc'd for C₃₉H₅₂N₈O₄S 728.4; found 729.3.

Step 2. A mixture of (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-11H-8-oxa-2 (4,2)-thiazola-1 (5,3)-indola-6 (1,3)-pyridazinacycloundecaphane-5,7-dione (6 g, 8.23 mmol) and lithium N-(dimethylcarbamoyl)-N-methyl-L-valinate (4.28 g, 20.58 mmol) in DMF (80 mL), was added DIEA (53.19 g, 411.55 mmol). The reaction mixture was stirred for 5 minutes, then added CIP (3.43 g, 12.35 mmol) in one portion. The resulting solution was stirred at 25° C. for 1 h, then quenched with water (100 mL), extracted with EtOAc (300 mL). The organic layer was separated and washed with saturated ammonium chloride aqueous solution (100 mL×3) and water (100 ml×2). The combined organic layers were concentrated under reduced pressure. The residue was purified by reverse phase chromatography to afford (2S)—N-((63S,4S,Z)-11-ethyl-12-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-3-methyl-2-(1,3,3-trimethylureido) butanamide (2.5 g, 33.2% yield) as a solid. ¹H NMR (400 MHZ, DMSO-d₆) δ 8.52-8.34 (m, 3H), 7.82 (s, 1H), 7.79-7.69 (m, 1H), 7.60-7.50 (m, 1H), 7.26-7.16 (m, 1H), 5.64-5.50 (m, 1H), 5.20-5.09 (m, 1H), 4.40-4.08 (m, 5H), 3.92-3.82 (m, 1H), 3.66-3.50 (m, 2H), 3.37-3.35 (m, 1H), 3.30-3.28 (m, 1H), 3.28-3.20 (m, 4H), 3.19-3.15 (m, 3H), 3.12-3.04 (m, 1H), 2.99-2.89 (m, 1H), 2.81 (s, 6H), 2.77 (s, 4H), 2.48-2.38 (m, 5H), 2.22 (s, 3H), 2.16-2.04 (m, 2H), 1.88-1.78 (m, 2H), 1.60-1.45 (m, 2H), 1.39-1.29 (m, 3H), 0.97-0.80 (m, 12H), 0.34 (s, 3H). LCMS (ESI): m/z [M+H] calc'd for C₄₈H₆₈N₁₀O₆S 912.5; found 913.6.

### Example A265. The synthesis of N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-4-methylpiperazine-1-carboxamide

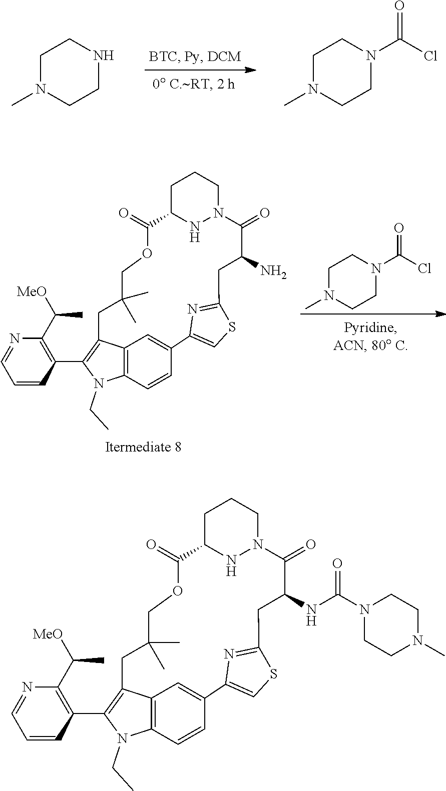

Step 1. To a stirred solution of 1-methylpiperazine (100 mg, 1.148 mmol) and Pyridine (275.78 mg, 3.44 mmol) in DCM (3 mL) were added BTC (112.5 mg, 0.38 mmol) in DCM (1 mL) dropwise at 0° C. under nitrogen atmosphere. The reaction was stirred for 2 hh 0° C. under nitrogen atmosphere. The resulting mixture was concentrated under reduced pressure to afford 4-methylpiperazine-1-carbonyl chloride (250 mg, crude) as an oil.

Step 2. To a stirred solution of Intermediate 8 (100 mg, 0.16 mmol) and pyridine (100 mg, 1.272 mmol) in ACN (2 mL) was added 4-methylpiperazine-1-carbonyl chloride (38.67 mg, 0.24 mmol) dropwise at 0° C. under nitrogen atmosphere. The reaction mixture was stirred for 2 hh at 0° C. under nitrogen atmosphere. The resulting mixture was diluted with water (100 mL) and extracted with EtOAc (100 mL×3). The combined organic layers were washed with brine (50 mL×3), dried over anhydrous Na₂SO₄, then filtered and concentrated under reduced pressure. The residue was purified by reverse flash chromatography to give N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-4-methylpiperazine-1-carboxamide (20 mg, 16.7% yield) as a solid. ¹H NMR (400 MHZ, DMSO-d₆) δ 8.76 (dd, J=4.8, 1.7 Hz, 1H), 8.50 (s, 1H), 8.14 (d, J=2.5 Hz, 1H), 7.79 (d, J=9.1 Hz, 2H), 7.77-7.72 (m, 1H), 7.58 (d, J=8.6 Hz, 1H), 7.52 (dd, J=7.7, 4.7 Hz, 1H), 6.82 (d, J=9.0 Hz, 1H), 5.32 (t, J=9.0 Hz, 1H), 4.99 (d, J=12.1 Hz, 1H), 4.43-4.02 (m, 5H), 3.57 (d, J=3.1 Hz, 2H), 3.26 (d, J=8.4 Hz, 6H), 2.97 (d, J=14.3 Hz, 1H), 2.80-2.66 (m, 1H), 2.55 (s, 1H), 2.40 (d, J=14.4 Hz, 1H), 2.32 (d, J=5.9 Hz, 4H), 2.21 (s, 3H), 2.09 (d, J=12.1 Hz, 1H), 1.77 (d, J=18.8 Hz, 2H), 1.52 (dd, J=11.8, 5.4 Hz, 1H), 1.37 (d, J=6.0 Hz, 3H), 1.24 (s, 1H), 0.90 (s, 3H), 0.85 (t, J=7.0 Hz, 3H), 0.32 (s, 3H). LCMS (ESI): m/z [M+H] calc'd for C₄₀H₅₂N₈O₅S 756.38; found 757.3.

### Example A598. The synthesis of (2S)—N-((6³S,3S,4S,Z)-1¹-ethyl-3-methoxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-3-methyl-2-(1,3,3-trimethylureido) butanamide

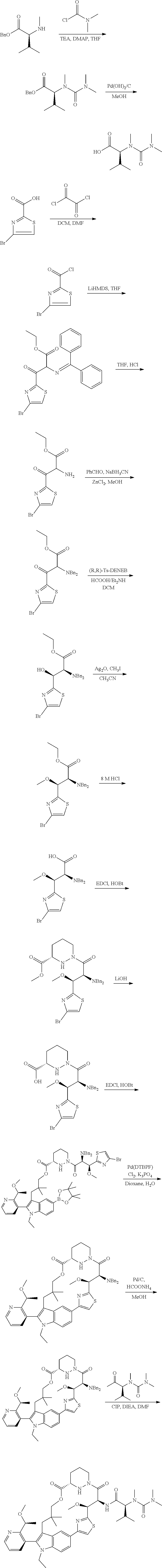

Step 1. A mixture of benzyl (2S)-3-methyl-2-(methylamino) butanoate (500 mg, 2.26 mmol) and dimethylcarbamyl chloride (1.215 g, 11.3 mmol) in THF (5 mL), was added TEA (2.286 g, 22.59 mmol) and DMAP (276.02 mg, 2.26 mmol) in portions under nitrogen atmosphere. The reaction mixture was stirred at 65° C. for 12 hh under nitrogen atmosphere, then quenched with water (100 mL) and was extracted with EtOAc (50 mL×3). The combined organic phase was dried over anhydrous sodium sulfate, filtered and concentrated under reduced pressure. The residue was purified by reverse phase chromatography to afford benzyl N-(dimethylcarbamoyl)-N-methyl-L-valinate (400 mg, 58.3% yield) as an oil. LCMS (ESI): m/z [M+H] calc'd for C₁₆H₂₄N₂O₃ 292.2; found 293.1.

Step 2. A mixture of benzyl N-(dimethylcarbamoyl)-N-methyl-L-valinate (400 mg, 1.37 mmol) and palladium hydroxide on carbon (400 mg, 2.85 mmol) in MeOH (10 mL) was stirred for 4 hh under hydrogen atmosphere. The reaction mixture was filtered and the filter cake was washed with MeOH (100 mL×3). The filtrate was concentrated under reduced pressure to afford N-(dimethylcarbamoyl)-N-methyl-L-valine (200 mg, crude) as an oil. LCMS (ESI): m/z [M+H] calc'd for C₉H₁₈N₂O₃ 202.1; found 203.1.

Step 3. A solution of 4-bromo-1,3-thiazole-2-carboxylic acid (10 g, 48.07 mmol) in DCM (100 mL), was added oxalyl chloride (16.27 mL, 192.28 mmol) and DMF (0.11 mL, 1.53 mmol) at 0° C. The reaction was stirred for at room temperature for 2 hh, then concentrated under reduced pressure to afford 4-bromo-1,3-thiazole-2-carbonyl chloride (10.8 g, crude).

Step 4. A solution of ethyl 2-[(diphenylmethylidene) amino]acetate (12.75 g, 47.69 mmol) in THF (100 mL) at −78° C., was added LiHMDS (47.69 mL, 47.69 mmol), and stirred at −40° C. for 30 minutes. Then the reaction mixture was added a solution of 4-bromo-1,3-thiazole-2-carbonyl chloride (10.8 g, 47.69 mmol) in THF (100 mL) at −78° C. and stirred at room temperature for 12 hh. The resulting mixture was quenched with water (100 mL), extracted with EtOAc (100 ml×3). The combined organic layers were dried over anhydrous sodium sulfate, filtered and concentrated under reduced pressure to afford ethyl 3-(4-bromothiazol-2-yl)-2-((diphenylmethylene) amino)-3-oxopropanoate (27 g, crude) as a solid. LCMS (ESI): m/z [M+H] calc'd for C₂₁H₁₇BrN₂O₃S 456.0; found 457.0.

Step 5. A solution of ethyl 3-(4-bromothiazol-2-yl)-2-((diphenylmethylene) amino)-3-oxopropanoate (20 g, 43.73 mmol) in THF (150 mL) at 0° C., was added 1 M HCl (100 mL) and stirred at room temperature for 2 hh. The resulting solution was concentrated and washed with ethyl ether (200 mL×2). The water phase was adjusted pH to 8 with sodium bicarbonate solution, then extracted with EtOAc (100 mL×3). The combined organic layers were dried over anhydrous sodium sulfate, filtered and concentrated under reduced pressure to afford ethyl 2-amino-3-(4-bromothiazol-2-yl)-3-oxopropanoate as an oil (9 g, crude). LCMS (ESI): m/z [M+H] calc'd for C₈H₉BrN₂O₃S 292.0; found 292.9.

Step 6. A solution of ethyl 2-amino-3-(4-bromothiazol-2-yl)-3-oxopropanoate (10 g, 34.11 mmol) in MeOH (200 mL) at 0° C., was added benzaldehyde (7.24 g, 68.23 mmol), zinc chloride (9.3 g, 68.23 mmol) and NaBH₃CN (4.29 g, 68.23 mmol). The reaction was stirred at room temperature for 2 hh, then quenched with water (100 mL) and concentrated. The resulting mixture was extracted with EtOAc (100 mL×3). The combined organic layers were dried over anhydrous sodium sulfate, filtered and concentrated under reduced pressure. The residue was purified by silica gel chromatography to afford ethyl 3-(4-bromothiazol-2-yl)-2-(dibenzylamino)-3-oxopropanoate as a solid (8.4 g, 52% yield). LCMS (ESI): m/z [M+H] calc'd for C₂₂H₂₁BrN₂O₃S 472.1; found 473.0.

Step 7. A mixture of ethyl 3-(4-bromothiazol-2-yl)-2-(dibenzylamino)-3-oxopropanoate (5 g, 10.56 mmol) and (R,R)-TS-DENEB (1.375 g, 2.11 mmol) in DCM (100 mL), was added HCOOH (1.99 mL, 43.29 mmol) and diethylamine (2.2 mL, 2.11 mmol) dropwise at room temperature under nitrogen atmosphere. The reaction mixture was stirred at 50° C. for 12 hh under nitrogen atmosphere. The resulting mixture was concentrated under reduced pressure. The residue was purified by silica gel column chromatography to afford ethyl (2S,3S)-3-(4-bromothiazol-2-yl)-2-(dibenzylamino)-3-hydroxypropanoate (3.148 g, 60% yield) as an oil. LCMS (ESI): m/z [M+H] calc'd for C₂₂H₂₃BrN₂O₃S 474.1; found 475.0.

Step 8. A mixture of ethyl (2S,3S)-3-(4-bromothiazol-2-yl)-2-(dibenzylamino)-3-hydroxypropanoate (1 g, 2.1 mmol) and Ag₂O (4.88 g, 21.06 mmol) in acetonitrile (10 mL), was added iodomethane (3.58 g, 25.22 mmol) in portions. The reaction mixture was stirred at 50° C. for 12 hh, then filtered. The filter cake was washed with MeOH (50 mL×2). The filtrate was concentrated under reduced pressure to afford ethyl (2S,3S)-3-(4-bromothiazol-2-yl)-2-(dibenzylamino)-3-methoxypropanoate (1.06 g, crude) as an oil. LCMS (ESI): m/z [M+H] calc'd for C₂₃H₂₅BrN₂O₃S 488.1; found 489.3.

Step 9. A mixture of ethyl (2S,3S)-3-(4-bromothiazol-2-yl)-2-(dibenzylamino)-3-hydroxypropanoate (1.06 g, 2.3 mmol) in HCl (10 ml, 8 M) was stirred at 80° C. for 12 hh and concentrated by reduced pressure. The residue was purified by reverse phase chromatography to afford (2S,3S)-3-(4-bromothiazol-2-yl)-2-(dibenzylamino)-3-methoxypropanoic acid (321 mg, 31.7% yield) as a solid. LCMS (ESI): m/z [M+H] calc'd for C₂₁H₂₁BrN₂O₃S 460.1; found 461.1.

Step 10. A solution of (2S,3S)-3-(4-bromothiazol-2-yl)-2-(dibenzylamino)-3-methoxypropanoic acid (4.61 g, 10 mmol) in DCM (100 mL) at 0° C. was added methyl (3S)-1,2-diazinane-3-carboxylate bis (trifluoroacetic acid) salt (3.72 g, 15 mmol), NMM (10.1 mL. 100 mmol), EDCl (3.8 g, 20 mmol) and HOBt (5.39 g, 39.89 mmol). The solution was warmed to room temperature and stirred for 1 h. The reaction was then quenched with H₂O (100 mL) and was extracted with EtOAc (100 mL×3). The combined organic layers were dried over anhydrous sodium sulfate, filtered, and concentrated under reduced pressured. The residue was purified by silica gel column chromatography to give methyl(S)-1-((2S,3S)-3-(4-bromothiazol-2-yl)-2-(dibenzylamino)-3-methoxypropanoyl) hexahydropyridazine-3-carboxylate (5.11 g, 90% yield) as a solid. LCMS (ESI): m/z [M+H] calc'd for C₂₇H₃₁BrN₄O₄S 587.1; found 586.1.

Step 11. A solution of methyl(S)-1-((2S,3S)-3-(4-bromothiazol-2-yl)-2-(dibenzylamino)-3-methoxypropanoyl) hexahydropyridazine-3-carboxylate (5.11 g, 9 mmol) in THF (100 mL)/H₂O (100 mL) was added LiOH (1.81 g, 45 mmol) under N₂ atmosphere and the resulting mixture was stirred for 2 hh at 25° C. The resulting mixture was concentrated under reduced pressure, the residue was acidified to pH 5 with HCL (1N). The aqueous layer was extracted with DCM (50 ml×3). The combined organic phase was concentrated under reduced pressure to give (S)-1-((2S,3S)-3-(4-bromothiazol-2-yl)-2-(dibenzylamino)-3-methoxypropanoyl) hexahydropyridazine-3-carboxylic acid (4.38 g, 85% yield) as a solid. LCMS (ESI): m/z [M+H] calc'd for C₂₆H₂₉BrN₄O₄S 572.1; found 573.1.

Step 12. A mixture of(S)-1-((2S,3S)-3-(4-bromothiazol-2-yl)-2-(dibenzylamino)-3-methoxypropanoyl) hexahydropyridazine-3-carboxylic acid (1.15 g, 2 mmol) and(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (985 mg, 2 mmol) in DCM (50 mL), was added DIEA (1.034 g, 8 mmol), EDCI (1.15 g, 558.3 mmol), HOBT (270.2 mg, 2 mmol). The reaction solution was stirred at 25° C. for 16 hh. The resulting mixture was diluted with DCM (200 mL), washed with water (50 mL×2) and brine (50 mL×3) and dried over anhydrous sodium sulfate, filtered and concentrated under reduced pressure. The residue was purified by silica gel column chromatography to afford 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-(S)-1-((2S,3S)-3-(4-bromothiazol-2-yl)-2-(dibenzylamino)-3-dimethylpropyl methoxypropanoyl) hexahydropyridazine-3-carboxylate (1.13 g, 54% yield) as a solid. LCMS (ESI): m/z [M+H] calc'd for C₅₅H₆₈BBrN₆O₇S 1046.4; found 1047.4.

Step 13. A mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((2S,3S)-3-(4-bromothiazol-2-yl)-2-(dibenzylamino)-3-methoxypropanoyl) hexahydropyridazine-3-carboxylate (250 mg, 0.24 mmol) and Pd(DtBPF)Cl₂ (15.55 mg, 0.024 mmol) in dioxane (5 mL) and water (1 mL), was added K₃PO₄ (126.59 mg, 0.6 mmol) in portions under nitrogen atmosphere. The reaction mixture was stirred at 80° C. for 2 hh under nitrogen atmosphere. The resulting mixture was diluted with water (20 mL) and extracted with EtOAc (10 mL×3), dried over anhydrous sodium sulfate, filtered and concentrated under reduced pressure. The residue was purified by silica gel column chromatography to afford (6³S,3S,4S,Z)-4-(dibenzylamino)-1¹-ethyl-3-methoxy-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (137 mg,44.38%) as a solid. LCMS (ESI): m/z [M+H] calc'd for C₄₉H₅₆N₆O₅S 840.4; found 841.5.

Step 14. A mixture of ((6³S,3S,4S,Z)-4-(dibenzylamino)-11-ethyl-3-methoxy-12-(2-((S)-1-methoxyethyl) pyridin-3-yl)-10, 10-dimethyl-6¹,6²,6³,6⁴,6⁵,6⁶-hexahydro-11H-8-oxa-2 (4,2)-thiazola-1 (5,3)-indola-6 (1,3)-pyridazinacycloundecaphane-5,7-dione (100 mg, 0.12 mmol) and Pd/C (253.06 mg, 2.38 mmol) in MeOH (10 mL), was added HCOONH₄ (149.94 mg, 2.38 mmol) in portions. The reaction mixture was stirred at 60° C. for 6 hh under hydrogen atmosphere. The resulting mixture was filtered, the filter cake was washed with MeOH (100 mL×10). The filtrate was concentrated under reduced pressure to afford (6³S,3S,4S,Z)-4-amino-1¹-ethyl-3-methoxy-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (56 mg, crude) as an oil. LCMS (ESI): m/z [M+H] calc'd for C₃₅H₄₄N₆O₅S 660.3; found 661.2.

Step 15. A mixture of (6³S,3S,4S,Z)-4-amino-11-ethyl-3-methoxy-12-(2-((S)-1-methoxyethyl) pyridin-3-yl)-10, 10-dimethyl-6¹,6²,6³,6⁴,6⁵,6⁶-hexahydro-11H-8-oxa-2 (4,2)-thiazola-1 (5,3)-indola-6 (1,3)-pyridazinacycloundecaphane-5,7-dione (56 mg, 0.085 mmol) and N-(dimethylcarbamoyl)-N-methyl-L-valine (51.42 mg, 0.25 mmol) in DMF (2 mL), was added 2-Chloro-1,3-dimethylimidazolidinium hexafluorophosphate (47.55 mg, 0.17 mmol) and DIEA (547.62 mg, 4.24 mmol) in portions. The reaction mixture was stirred for 12 hh. The resulting mixture was purified by reverse phase chromatography to afford (2S)—N-((6³S,3S,4S,Z)-1¹-ethyl-3-methoxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-3-methyl-2-(1,3,3-trimethylureido) butanamide (1.5 mg, 2.06% yield) as a solid. ¹H NMR (400 MHZ, Methanol-d₄) δ 8.74-8.77 (m, 1H), 8.61 (d, J=1.6 Hz, 1H), 7.99-7.87 (m, 1H), 7.73-7.66 (m, 1H), 7.68 (s, 1H), 7.60-7.55 (m, 1H), 7.49 (d, J=8.7 Hz, 1H), 7.31 (d, J=51.0 Hz, OH), 5.89 (s, 1H), 4.95 (s, 1H), 4.43 (d, J=13.0 Hz, 1H), 4.36 (q, J=6.2 Hz, 1H), 4.33-4.19 (m, 2H), 4.10-4.03 (m, 1H), 4.03 (d, J=11.2 Hz, 1H), 3.78-3.67 (m, 2H), 3.65 (s, OH), 3.46 (s, 3H), 3.34 (s, 4H), 3.01 (d, J=10.3 Hz, 1H), 2.93 (s, 6H), 2.88-2.81 (m, 1H), 2.78 (s, 3H), 2.70-2.60 (m, 1H), 2.23-2.01 (m, 2H), 2.03 (s, OH), 1.99 (d, J=13.3 Hz, 1H), 1.91-1.74 (m, 1H), 1.69-1.54 (m, 1H), 1.45 (d, J=6.2 Hz, 3H), 1.37-1.32 (m, 1H), 1.28 (s, 1H), 0.94 (p, J=6.7 Hz, 12H), 0.51 (s, 3H), 0.10 (s, 1H). LCMS (ESI): m/z [M+H] calc'd for C₄₄H₆₀N₈O₇ 844.4; found 845.4.

### Example A286. The synthesis of (1S,2S)—N-((6³S,4S,Z)-1¹-ethyl-1²-(2-((S)-1-methoxyethyl) pyridin-3-yl)-64,10, 10-trimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

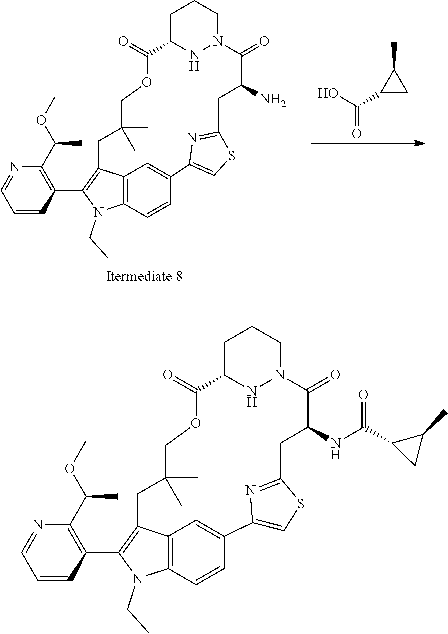

Step 1. A solution of Intermediate 8 (8 g, 10.95 mmol) in HCl (200 mL, 4M in 1,4-dioxane) was stirred at 0° C. for 2 hh, then concentrated under reduced pressure. The resulting mixture was diluted with DCM (60 mL) and saturated NaHCO₃ aqueous solution (40 mL). The organic phase was separated and washed with brine (50 mL×2) and concentrated under reduced pressure to give (6³S,4S,Z)-4-amino-11-ethyl-12-(2-((S)-1-methoxyethyl) pyridin-3-yl)-10,10-dimethyl-61,62,63,64,65,66-hexahydro-11H-8-oxa-2 (4,2)-thiazola-1 (5,3)-indola-6 (1,3)-pyridazinacycloundecaphane-5,7-dione (10.3 g, crude) as solid. LCMS (ESI): m/z [M+H] calc'd for C₃₄H₄₂N₆O₄S 630.3; found 631.2.

Step 2. A stirred solution of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)- pyridazinacycloundecaphane-5,7-dione (8 g, 12.68 mmol) in DMF (50 mL) at 0° C., was added DIEA (9.83 g, 76.09 mmol), (1S,2S)-2-methylcyclopropane-1-carboxylic acid (1.52 g, 15.22 mmol) and HATU (14.47 g, 38.05 mmol). The reaction mixture was stirred at 0° C. for 2 hh and concentrated under reduced pressure. The residue was purified by reverse phase chromatography to afford (1S,2S)—N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide (6.84 g, 56.37% yield) as a solid. ¹H NMR (400 MHZ, DMSO-d₆) δ 8.79 (dd, J=4.7, 1.9 Hz, 1H), 8.59-8.40 (m, 2H), 7.95-7.86 (m, 1H), 7.82-7.71 (m, 2H), 7.66-7.53 (m, 2H), 5.57 (t, J=9.0 Hz, 1H), 5.07 (s, 1H), 4.41-4.28 (m, 2H), 4.25 (d, J=12.4 Hz, 1H), 4.17 (d, J=10.8 Hz, 1H), 4.09 (d, J=7.2 Hz, 1H), 3.58 (s, 2H), 3.32 (d, J=14.6 Hz, 1H), 3.28 (s, 3H), 3.16 (dd, J=14.7, 9.1 Hz, 1H), 2.95 (d, J=14.4 Hz, 1H), 2.75 (m, J=12.1, 7.1 Hz, 1H), 2.43 (d, J=14.4 Hz, 1H), 2.13-2.00 (m, 1H), 1.76 (d, J=22.0 Hz, 2H), 1.60-1.44 (m, 2H), 1.38 (d, J=6.1 Hz, 3H), 1.07 (d, J=1.9 Hz, 4H), 0.86 (dd, J=14.1, 7.1 Hz, 7H), 0.59-0.49 (m, 1H), 0.34 (s, 3H). LCMS (ESI): m/z [M+H] calc'd for C₃₉H₄₈N₆O₅S 712.3; found 713.2.

### Example A613. The synthesis of N-((2S)-1-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) amino)-3-methyl-1-oxobutan-2-yl)-3-methoxy-N-methylazetidine-1-carboxamide

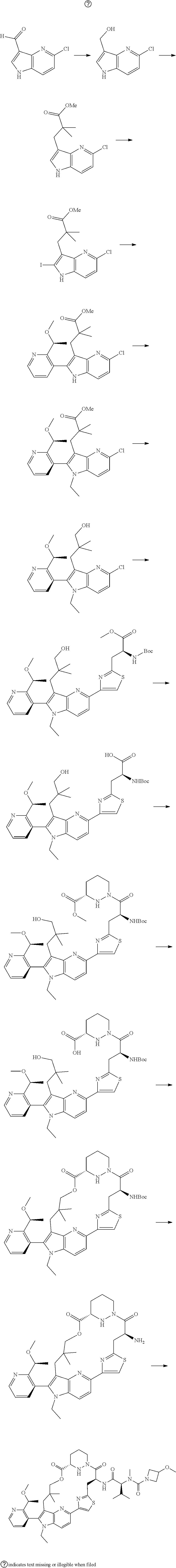

Step 1. A mixture of methyl(S)-3-(4-bromothiazol-2-yl)-2-((tert-butoxycarbonyl) amino) propanoate (920 mg, 2.5 mmol), 4,4,5,5-tetramethyl-2-(4,4,5,5-tetramethyl-1,3,2-dioxaborolan-2-yl)-1,3,2-dioxaborolane (1.6 g, 6.3 mmol), x-Phos (180 mg, 0.5 mmol), Pd₂(dba)₃-chloroform (130 mg, 0.13 mmol) and potassium acetate (740 mg, 7.5 mmol) in dioxane (25 mL) in a sealed tube under N₂ atmosphere, was stirred at 110° C. for 8 hh to afford crude methyl(S) -2-((tert-butoxycarbonyl) amino)-3-(4-(4,4,5,5-tetramethyl-1,3,2-dioxaborolan-2-yl) thiazol-2-yl) propanoate as a solution. LCMS (ESI): m/z [M+H] calc'd for C₁₈H₂₉BN₂O₆S 412.2; found 331.1.

Step 2. A mixture of 5-chloro-1H-pyrrolo [3,2-b]pyridine-3-carbaldehyde (7 g, 39 mmol) in MeOH (140 mL) under N₂ atmosphere, was added NaBH₄ (2.9 g, 78 mmol) at 0° C. The reaction mixture was stirred at 10° C. for 2 hh and concentrated under reduced pressure. The residue was diluted with EtOAc (200 mL), washed with brine (25 mL), dried over anhydrous sodium sulfate and concentrated under reduced pressure. The residue was purified by silica gel column chromatography to afford (5-chloro-1H-pyrrolo [3,2-b]pyridin-3-yl) methanol (3.5 g, 55% yield) as a solid. LCMS (ESI): m/z [M+H] calc'd for C₈H₇ClN₂O 182.0; found 183.0.

Step 3. A mixture of (5-chloro-1H-pyrrolo [3,2-b]pyridin-3-yl) methanol (3.5 g, 19 mmol) and ((1-methoxy-2-methylprop-1-en-1-yl)oxy) trimethylsilane (6.7 g, 38 mmol) in THF (50 mL), was dropwise added TMSOTf (3.8 g, 17.1 mmol) at 0° C. The reaction mixture was stirred at 5° C. for 2 hh, then diluted with EtOAc (100 mL), washed with saturated NaHCO₃ aqueous (50 mL), and brine (50 mL×2). The organic layer was dried over anhydrous sodium sulfate and concentrated under reduced pressure. The residue was purified by silica gel column chromatography to afford methyl 3-(5-chloro-1H-pyrrolo [3,2-b]pyridin-3-yl)-2,2-dimethylpropanoate (3 g, 59% yield) as a solid. LCMS (ESI): m/z [M+H] calc'd for C₁₃H₁₅ClN₂O₂ 266.1; found 267.1.

Step 4. A mixture of methyl 3-(5-chloro-1H-pyrrolo [3,2-b]pyridin-3-yl)-2,2-dimethylpropanoate (3 g, 11 mmol) in anhydrous THF (50 mL) at 0° C., was added AgOTf (4.3 g, 17 mmol) and 12 (2.9 g, 11 mmol). The reaction mixture was stirred at 0° C. for 2 hh, then quench with conc. Na₂SO₃ (20 mL), diluted with EtOAc (50 mL) and filtered. The filtrate was washed with brine (50 mL). The organic layer was dried over anhydrous sodium sulfate and concentrated under reduced pressure. The residue was purified with silica gel column chromatography to afford methyl 3-(5-chloro-2-iodo-1H-pyrrolo [3,2-b]pyridin-3-yl)-2,2-dimethylpropanoate (2.3 g, 52% yield) as a solid. LCMS (ESI): m/z [M+H] calc'd for C₁₃H₁₄ClIN₂O₂ 393.0; found 392.0 .

Step 5. A mixture of methyl 3-(5-chloro-2-iodo-1H-pyrrolo [3,2-b]pyridin-3-yl)-2,2-dimethylpropanoate (2.3 g, 5.9 mmol), 2-(2-(2-methoxyethyl)phenyl)-4,4,5,5-tetramethyl-1,3,2-dioxaborolane (1.6 g, 7.1 mmol) and K₂CO₃ (2.4 g, 18 mol) in dioxane (25 mL) and water (5 mL) under N₂ atmosphere, was added Pd(dppf)Cl₂·DCM (480 mg, 0.59 mmol). The reaction mixture was stirred at 70° C. for 4 hh, then diluted with EtOAc (200 mL) and washed with brine (25 mL). The separated organic layer was dried over anhydrous sodium sulfate and concentrated under reduced pressure. The residue was purified by silica gel column chromatography to afford methyl(S)-3-(5-chloro-2-(2-(1-methoxyethyl) pyridin-3-yl)-1H-pyrrolo [3,2-b]pyridin-3-yl)-2,2-dimethylpropanoate (2 g, yield 84%) as a solid. LCMS (ESI): m/z [M+H] calc'd for C₂₁H₂₄ClN₃O₃ 401.2; found 402.2.

Step 6. A mixture of methyl(S)-3-(5-chloro-2-(2-(1-methoxyethyl) pyridin-3-yl)-1H-pyrrolo [3,2-b]pyridin-3-yl)-2,2-dimethylpropanoate (2 g, 5 mmol), cesium carbonate (3.3 g, 10 mmol) and EtI (1.6 g, 10 mmol) in DMF (30 mL) was stirred at 25° C. for 10 hh. The resulting mixture was diluted with EtOAc (100 mL), washed with brine (20 mL×4). The separated organic layer was dried over anhydrous sodium sulfate, filtered and concentrated under reduced pressure. The residue was purified by silica gel column chromatography to afford methyl(S)-3-(5-chloro-1-ethyl-2-(2-(1-methoxyethyl) pyridin-3-yl)-1H-pyrrolo [3,2-b]pyridin-3-yl)-2,2-dimethylpropanoate as two diastereomers (P1: 0.7 g, 32% yield; P2: 0.6 g, 28% yield) both as a solid. LCMS (ESI): m/z [M+H] calc'd for C₂₃H₂₈ClN₃O₃ 429.2; found 430.2.

Step 7. A mixture of methyl(S)-3-(5-chloro-1-ethyl-2-(2-(1-methoxyethyl) pyridin-3-yl)-1H-pyrrolo [3,2-b]pyridin-3-yl)-2,2-dimethylpropanoate (P2, 1.2 g, 2.8 mmol) in anhydrous THF (20 mL) at 5° C., was added LiBH₄ (120 mg, 5.6 mmol). The reaction mixture was stirred at 60° C. for 4 hh, then quenched with conc. NH₄Cl (20 mL), diluted with EtOAc (50 mL) and washed with brine (30 mL). The organic layer was separated, dried over anhydrous sodium sulfate and concentrated under reduced pressure. The residue was purified with silica gel column chromatography to afford(S)-3-(5-chloro-1-ethyl-2-(2-(1-methoxyethyl) pyridin-3-yl)-1H-pyrrolo [3,2-b]pyridin-3-yl)-2,2-dimethylpropan-1-ol (1 g, 89% yield) as a solid. LCMS (ESI): m/z [M+H] calc'd for C₂₂H₂₈ClN₃O₂ 401.2; found 402.2.

Step 8. A mixture of solution from Step 1 (360 mg, crude, 1 mmol) in dioxane (10 mL) and water (2 mL), was added(S)-3-(5-chloro-1-ethyl-2-(2-(1-methoxyethyl) pyridin-3-yl)-1H-pyrrolo [3,2-b]pyridin-3-yl)-2,2-dimethylpropan-1-ol (200 mg, 0.5 mmol), potassium carbonate (200 mg, 1.5 mmol) and Pd-118 (30 mg, 0.05 mmol). This reaction mixture was stirred at 70° C. for 3 hh, then diluted with EtOAc (40 mL), filtered. The filtrate was washed with brine, dried over anhydrous sodium sulfate, filtered and concentrated under reduced pressure. The residue was purified with silica gel column chromatography to afford methyl(S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoate (300 mg, 65% yield) as a solid. LCMS (ESI): m/z [M+H] calc'd for C₃₄H₄₅N₅O₆S 651.3; found 652.3.

Step 9. A solution of methyl(S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoate (280 mg, 0.43 mmol) in MeOH (4 mL), was added a solution of lithium hydroxide (51 mg, 2.15 mmol) in water (2 mL) at 20° C. The reaction was stirred at 20° C. for 5 hh, then adjusted to pH=3˜4 with HCl (1 N). The resulting mixture was diluted with water (30 mL) and extracted with EtOAc (15 mL×3). The combined organic phase was washed with brine (10 mL), dried over anhydrous sodium sulfate, filtered and concentrated under reduced pressure to give (S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoic acid (280 mg, crude) as a solid. LCMS (ESI): m/z [M+H] calc'd for C₃₃H₄₃N₅O₆S 637.3; found 638.3.

Step 10. A solution of(S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoic acid (274 mg, 0.43 mmol) and methyl(S)-hexahydropyridazine-3-carboxylate (280 mg, 0.64 mmol) in DMF (3 mL) at 5° C., was added a solution of HATU (245 mg, 0.64 mmol) and DIEA (555 mg, 4.3 mmol) in DMF (2 mL). The reaction was stirred for 1 h, then diluted with EtOAc (20 mL) and water (20 mL). The organic layer was separated and washed with water (20 mL×3) and brine (20 mL), dried over anhydrous sodium sulfate, filtered concentrated under reduced pressure. The residue was purified by silica gel chromatography to give methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoyl) hexahydropyridazine-3-carboxylate (230 mg, 70% yield) as solid. LCMS (ESI): m/z [M+H] calc'd for C₃₉H₅₃N₇O₇S 763.4; found 764.3.

Step 11. A solution of methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoyl) hexahydropyridazine-3-carboxylate (230 mg, 0.3 mmol) in DCE (3 mL), was added trimethyltin hydroxide (300 mg, 1.4 mmol) under N₂ atmosphere. The reaction was stirred at 65° C. for 16 hh, then concentrated under reduced pressure. The residue was diluted with EtOAc (20 mL), washed with water (20 mL) and brine (10 mL), dried over anhydrous sodium sulfate, filtered and concentrated under reduced pressure to afford(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoyl) hexahydropyridazine-3-carboxylic acid (200 mg, crude) as foam. LCMS (ESI): m/z [M+H] calc'd for C₃₈H₅₁N₇O₇S 749.4; found 750.3.

Step 12. A solution of(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoyl) hexahydropyridazine-3-carboxylic acid (245 mg, 0.32 mmol) in DCM (50 mL) at 5° C., were added HOBt (432 mg, 3.2 mmol), EDCl (1.8 g, 9.6 mmol) and DIEA (1.65 g, 12.8 mmol). The reaction mixture was stirred at 20° C. for 16 hh, then concentrated under reduced pressure. The residue was diluted with EtOAc (20 mL) and water (20 mL). The organic layer was separated and washed with water (30 mL×3) and brine (30 mL), dried over anhydrous sodium sulfate, filtered and concentrated under reduced pressure. The residue was purified by silica gel chromatography to give tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) carbamate (100 mg, 43% yield) as solid. LCMS (ESI): m/z [M+H] calc'd for C₃₈H₄₉N₇O₆S 731.4; found 732.3.

Step 13. A solution of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) carbamate (80 mg, 0.11 mmol) in TFA (0.2 mL) and DCM (0.6 mL) was stirred at 20° C. for 1 h. The reaction was concentrated to afford (6³S,4S,Z)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-5,7-dione (72 mg, 95% yield) as a solid. LCMS (ESI): m/z [M+H] calc'd for C₃₃H₄₁N₇O₄S 631.3; found 632.3.

Step 14. A solution of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-5,7-dione (100 mg, 0.16 mmol) and (2S)-2-[(3-methoxyazetidin-1-yl) carbonyl (methyl) amino]-3-methylbutanoic acid (78 mg, 0.32 mmol) in DMF (5 mL) at 0° C., was dropwise added a solution of DIEA (620 mg, 4.8 mmol) and HATU (91 mg, 0.24 mmol) in DMF (5 mL). The reaction mixture was stirred at 0° C. for 2 hh, then diluted with EtOAc (50 mL), washed with water (25 mL×3), dried over anhydrous sodium sulfate, filtered and concentrated under reduced pressure. The residue was purified by silica gel column chromatography to afford N-((2S)-1-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) amino)-3-methyl-1-oxobutan-2-yl)-3-methoxy-N-methylazetidine-1-carboxamide (112.9 mg, 82% yield) as a solid. ¹H NMR (400 MHZ, CD₃OD) δ 8.77-8.75 (dd, J=4.8, 1.7 Hz, 1H), 7.96-7.94 (d, J=8.6 Hz, 1H), 7.89-7.87 (dd, J=8.4, 2.3 Hz, 2H), 7.77-7.74 (d, J=8.6 Hz, 1H), 7.58-7.55 (dd, J=7.8, 4.8 Hz, 1H), 5.73-5.70 (dd, J=8.0, 2.7 Hz, 1H), 4.41-4.38 (dt, J=8.5, 4.3 Hz, 2H), 4.33-4.26 (m, 3H), 4.24-4.17 (m, 3H), 4.04-4.01 (dd, J=11.9, 3.0 Hz, 1H), 3.99-3.96 (m, 1H), 3.89-3.83 (m, 2H), 3.53-3.49 (dd, J=9.7, 7.3 Hz, 2H), 3.46-3.45 (d, J=3.0 Hz, 1H), 3.35 (s, 3H), 3.34-3.33 (d, J=4.5 Hz, 3H), 3.28 (s, 1H), 2.89 (s, 3H), 2.78-2.71 (td, J=13.2, 3.4 Hz, 1H), 2.52-2.48 (d, J=14.1 Hz, 1H), 2.23-2.20 (m, 1H), 2.19-2.11 (d, J=10.2 Hz, 1H), 1.91-1.88 (d, J=13.5 Hz, 1H), 1.73-1.70 (dd, J=9.0, 3.9 Hz, 1H), 1.56-1.50 (m, 1H), 1.47-1.46 (d, J=6.1 Hz, 3H), 0.98-0.91 (m, 9H), 0.88 (s, 3H), 0.45 (s, 3H). LCMS (ESI): m/z [M+H] calc'd for C₄₄H₅₉N₉O₇S 857.4; found 858.3.

### Example A579. The synthesis of N-((2S)-1-(((6³S,6⁴S,4S,Z)-1¹-ethyl-1²-(2-((S)-1-methoxyethyl) pyridin-3-yl)-64,10,10-trimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-3-methoxy-N-methylazetidine-1-carboxamide

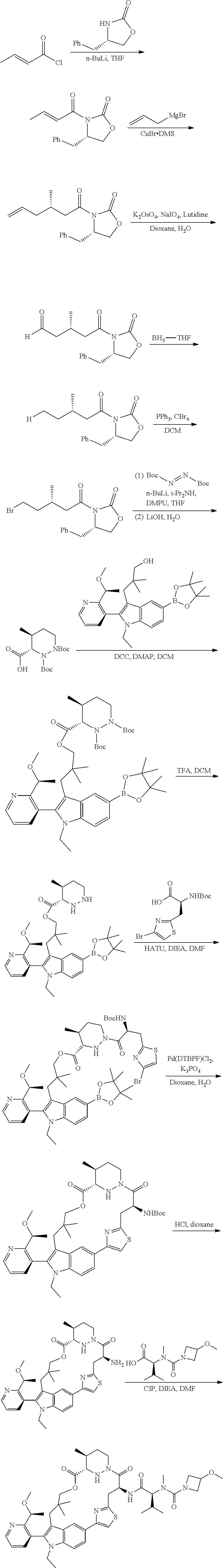

Step 1. A solution of(S)-4-benzyloxazolidin-2-one (10 g, 56.43 mmol) in THF (100 mL) was purged with nitrogen, was added of n-butyllithium (24.83 mL, 62.08 mmol) at −78° C. under nitrogen atmosphere, then stirred for at −78° C. for 15 minutes. The reaction mixture was added 2-butenoyl chloride (6.49 g, 62.08 mmol). The resulting solution was stirred at −78° C. for 30 minutes, then slowly warmed up to 0° C. and stirred for 15 minutes, quenched with saturated ammonium chloride solution (100 mL). The resulting solution was extracted with EtOAc (100 mL×3) and the combined organic phase was dried over anhydrous sodium sulfate and concentrated under reduced pressure. The residue was purified by silica gel column chromatography to afford (4S)-4-benzyl-3-[(2E)-but-2-enoyl]-1,3-oxazolidin-2-one (12.26 g, 88.57% yield) as an oil. LCMS (ESI): m/z [M+H] calc'd for C₁₄H₁₅NO₃ 245.1; found 246.1.

Step 2. A solution of CuBr·DMS (12.07 g, 58.71 mmol) in THF (120 mL) was purged and maintained nitrogen atmosphere, added of allylmagnesium bromide (58.71 mL, 58.71 mmol) at −78° C. The reaction was stirred at −60° C. for 30 minutes under nitrogen atmosphere followed by addition of (4S)-4-benzyl-3-[(2E)-but-2-enoyl]-1,3-oxazolidin-2-one (12 g, 48.92 mmol) at −78° C. The resulting solution was stirred at −50° C. for 3 more hh, then quenched with saturated ammonium chloride solution (100 mL) and extracted with EtOAc (60 mL×3). The combined organic phase was dried over anhydrous sodium sulfate and concentrated under reduced pressure. The residue was purified by silica gel column chromatography to afford(S)-4-benzyl-3-((S)-3-methylhex-5-enoyl) oxazolidin-2-one (13.2 g, 93.89% yield) as an oil. LCMS (ESI): m/z [M+H] calc'd for C₁₇H₂₁NO₃ 287.2; found 288.2.

Step 3. A solution of(S)-4-benzyl-3-((S)-3-methylhex-5-enoyl) oxazolidin-2-one (13.2 g, 45.94 mmol) in dioxane (200 mL) and water (200 mL), was added 2,4-Lutidine (9.84 g, 91.87 mmol) followed with K₂OsO₄·2H₂O (1.69 g, 4.59 mmol) at 0° C. The reaction solution was stirred at 0° C. for 15 minutes, then was added NaIO₄ (39.3 g, 183.74 mmol). The resulting mixture was stirred at 0° C. for 1 h, then extracted with EtOAc (150 mL×3). The combined organic phase was hydrochloric acid (100 mL×3), dried over anhydrous sodium sulfate and concentrated under reduced pressure to afford(S)-5-((S)-4-benzyl-2-oxooxazolidin-3-yl)-3-methyl-5-oxopentanal (12.3 g, crude) as an oil. LCMS (ESI): m/z [M+H] calc'd for C₁₆H₁₉NO₄ 289.1; found 290.1.

Step 4. A solution of(S)-5-((S)-4-benzyl-2-oxooxazolidin-3-yl)-3-methyl-5-oxopentanal (12.3 g, 42.51 mmol) in THF (200 mL) was purged and maintained with nitrogen atmosphere, then added borane-tetrahydrofuran complex (55.27 mL, 55.27 mmol) at 0° C. The reaction was stirred at 0° C. for 30 minutes, then quenched with methanol (40 mL). The resulting mixture was concentrated under reduced pressure. The residue was purified by silica gel column chromatography to afford(S) -4-benzyl-3-((S)-5-hydroxy-3-methylpentanoyl) oxazolidin-2-one (9.6 g, 77.51% yield) as an oil. LCMS (ESI): m/z [M+H] calc'd for C₁₆H₂₁NO₄ 291.1; found 292.1.

Step 5. A solution of(S)-4-benzyl-3-((S)-5-hydroxy-3-methylpentanoyl) oxazolidin-2-one (9.6 g, 32.95 mmol) and CBr₄ (16.39 g, 49.43 mmol) in DCM (120 mL) at 0° C., was added triphenylphosphine (12.96 g, 49.41 mmol). The reaction was stirred at 0° C. for 1 h, then quenched with ice water (100 mL) and extracted with DCM (100 mL×3). The combined organic phase was dried over anhydrous sodium sulfate and concentrated under reduced pressure. The residue was purified by silica gel column chromatography to afford(S)-4-benzyl-3-((R)-5-bromo-3-methylpentanoyl) oxazolidin-2-one (10 g, 85.67% yield) as an oil. LCMS (ESI): m/z [M+H] calc'd for C₁₆H₂₀BrNO₄ 353.1; found 354.1 .

Step 6. A mixture of n-BuLi (2.26 mL, 5.65 mmol) and diisopropylamine (571.3 mg, 5.65 mmol) in THF (10 mL) under nitrogen at −78° C., was added a cooled (−78° C.) solution of(S)-4-benzyl-3-((R)-5-bromo-3-methylpentanoyl) oxazolidin-2-one (2 g, 5.65 mmol) in THF (9 mL). The reaction mixture was stirred at −78° C. for 30 minutes, then was added a solution of (E)-N-[(tert-butoxycarbonyl) imino](tert-butoxy) formamide (1.3 g, 5.65 mmol) in THF (10 mL), stirred for another 30 minutes at −78° C. The resulting mixture was added DMPU (16 mL, 132.82 mmol) and warmed up to 0° C. and stirred for 90 minutes, followed by addition of a solution of LiOH·H₂O (1.18 g, 28.12 mmol) in water (20 mL). Then THF was removed under reduced pressure. The residue was washed with DCM (80 mL×3). The aqueous phase was acidified to pH 5˜6 with HCl (aq.), extracted with mixture of DCM/methanol (80 mL×3, 10:1). The combined organic layers were dried over anhydrous sodium sulfate, filtered, concentrated under reduced pressure. The residue was purified by reverse phase chromatography to afford (3S,4S)-1,2-bis (tert-butoxycarbonyl)-4-methylhexahydropyridazine-3-carboxylic acid (296 mg, 15.22% yield) as a solid. LCMS (ESI): m/z [M−H] calc'd for C₁₆H₂₈N₂O₆ 344.2; found 343.1.

Step 7. A mixture of (3S,4S)-1,2-bis (tert-butoxycarbonyl)-4-methylhexahydropyridazine-3-carboxylic acid (289 mg, 0.84 mmol) and(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (413.24 mg, 0.84 mmol) in DMF (10 mL) at 0° C., was added DMAP (51.26 mg, 0.42 mmol) and DCC (692.53 mg, 3.36 mmol). The reaction solution was stirred at room temperature for 1 h, then quenched with water/ice (10 mL), extracted with EtOAc (15 mL×3). The combined organic layers were washed with brine (50 mL×3), dried over anhydrous sodium sulfate and concentrated under reduced pressure. The residue was purified by silica gel column chromatography to afford 1,2-di-tert-butyl 3-(3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl) (3S,4S)-4-methyltetrahydropyridazine-1,2,3-tricarboxylate (538 mg, 78.3% yield) as a solid. LCMS (ESI): m/z [M−H] calc'd for C₄₅H₆₇BN₄O₉ 818.5; found 819.4.

Step 8. A solution of 1,2-di-tert-butyl 3-(3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl) (3S,4S)-4-methyltetrahydropyridazine-1,2,3-tricarboxylate (508 mg, 0.62 mmol) in DCM (25 mL), was added TFA (25 mL) at 0° C. The reaction solution was stirred at room temperature for 1 h. The resulting mixture was concentrated to afford 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl (3S,4S)-4-methylhexahydropyridazine-3-carboxylate (508 mg, crude) as an oil. LCMS (ESI): m/z [M−H] calc'd for C₃₅H₅₁BN₄O₅ 618.4; found 619.3.

Step 9. A solution of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl (3S,4S)-4-methylhexahydropyridazine-3-carboxylate (508 mg, 0.82 mmol) and(S)-3-(4-bromothiazol-2-yl)-2-((tert-butoxycarbonyl) amino) propanoic acid (288.41 mg, 0.82 mmol) in DMF (50 mL) at 0° C., was added DIEA (1061.31 mg, 8.21 mmol), HATU (468.35 mg, 1.23 mmol). The reaction solution was stirred at room temperature for 1 h, then quenched with ice water (30 mL) and extracted with EtOAc (30 mL×3). The combined organic phase was washed with brine (50 mL×3), dried over anhydrous sodium sulfate and concentrated under reduced pressure. The residue was purified by silica gel column chromatography to afford 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl (3S,4S)-1-((S)-3-(4-bromothiazol-2-yl)-2-((tert-butoxycarbonyl) amino) propanoyl)-4-methylhexahydropyridazine-3-carboxylate (431 mg, 55.14% yield) as a solid. LCMS (ESI): m/z [M−H] calc'd for C₄₆H₆₄BBrN₆O₈S 950.4; found 951.3.

Step 10. A mixture of Pd(DTBpf)Cl₂ (27.39 mg, 0.042 mmol) and K₃PO4 (89.2 mg, 0.42 mmol) in dioxane (5 mL) and water (1 mL) was purged nitrogen, stirred at 60° C. for 5 minutes under nitrogen atmosphere, then added a solution of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl (3S,4S)-1-((S)-3-(4-bromothiazol-2-yl)-2-((tert-butoxycarbonyl) amino) propanoyl)-4-methylhexahydropyridazine-3-carboxylate (200 mg, 0.21 mmol) in dioxane (5 mL) and water (1 mL) at 60° C. The reaction mixture was stirred at 60° C. for 1 h, then quenched with ice water (5 mL), extracted with EtOAc (15 mL×3). The combined organic layers were dried over anhydrous sodium sulfate and concentrated under reduced pressure. The residue was purified by silica gel column chromatography to afford tert-butyl ((6³S,6⁴S,4S,Z)-1¹-ethyl-1²-(2-((S)-1-methoxyethyl) pyridin-3-yl)-64,10,10-trimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate (70 mg, 44.72% yield) as a solid. LCMS (ESI): m/z [M−H] calc'd for C₄₀H₅₂N₆O₆S 744.4; found 745.4.

Step 11. A solution of tert-butyl ((6³S,6⁴S,4S,Z)-1¹-ethyl-1²-(2-((S)-1-methoxyethyl) pyridin-3-yl)-64,10,10-trimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (70 mg, 0.094 mmol) in dioxane (5 mL), was added HCl in dioxane (5 mL, 4M). The reaction was stirred at room temperature for 1 h, then concentrated under reduced pressure to afford (6³S,6⁴S,4S,Z)-4-amino-1¹-ethyl-1²-(2-((S)-1-methoxyethyl) pyridin-3-yl)-6⁴, 10, 10-trimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (124 mg, crude) as an oil. LCMS (ESI): m/z [M−H] calc'd for C₃₆H₄₅N₅O₄S 644.3; found 645.3.

Step 12. A mixture of (6³S,6⁴S,4S,Z)-4-amino-1¹-ethyl-1²-(2-((S)-1-methoxyethyl) pyridin-3-yl)-6⁴,10,10-trimethyl-6¹,6²,6³,6⁴,6⁵,6⁶-hexahydro-11H-8-oxa-2 (4,2)-thiazola-1 (5,3)-indola-6 (1,3)- pyridazinacycloundecaphane-5,7-dione (112 mg, 0.17 mmol) and N-(3-methoxyazetidine-1-carbonyl)-N-methyl-L-valine (50.92 mg, 0.21 mmol) in DMF (3 mL) at 0° C., was added DIEA (1.795 g, 13.9 mmol), 2-chloro-1,3-dimethylimidazolidinium hexafluorophosphate (72.57 mg, 0.26 mmol). The reaction was stirred at room temperature for 1 h and then filtered. The filtrate was purified by reverse phase chromatography to afford N-((2S)-1-(((6³S,6⁴S,4S,Z)-1¹-ethyl-1²-(2-((S)-1-methoxyethyl) pyridin-3-yl)-6⁴, 10, 10-trimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-3-methoxy-N-methylazetidine-1-carboxamide (25.6 mg, 16.92% yield) as a solid. ¹H NMR (400 MHZ, DMSO-d₆) δ 8.76 (dd, J=4.7, 1.8 Hz, 1H), 8.60 (s, 1H), 8.30-8.20 (m, 1H), 7.86-7.70 (m, 3H), 7.61-7.50 (m, 2H), 5.57-5.43 (m, 1H), 5.07 (d, J=12.1 Hz, 1H), 4.39-4.21 (m, 3H), 4.20-4.01 (m, 5H), 3.96 (d, J=11.1 Hz, 1H), 3.82 (dd, J=8.9, 3.6 Hz, 1H), 3.77-3.71 (m, 1H), 3.63-3.55 (m, 2H), 3.35-3.27 (m, 2H), 3.24 (s, 3H), 3.23-3.14 (m, 4H), 2.93-2.79 (m, 2H), 2.70 (s, 3H), 2.15-2.01 (m, 1H), 1.83-1.61 (m, 2H), 1.38 (d, J=6.1 Hz, 4H), 0.98 (d, J=6.4 Hz, 3H), 0.94-0.85 (m, 6H), 0.85-0.72 (m, 6H), 0.43 (s, 3H). LCMS (ESI): m/z [M−H] calc'd for C₄₆H₆₂N₈O₇S 870.4; found 871.4.

The following table of compounds (Table 3) were prepared using the aforementioned methods or variations thereof, as is known to those of skill in the art.

TABLE 3
Exemplary Compounds Prepared by Methods of the Present Invention

| | LCMS (ESI): m/z | | LCMS (ESI): m/z |
|---|---|---|---|
| Ex# | [M + H] Found | Ex# | [M + H] Found |
| A1 | 907.5 | A38 | 835.0 |
| A2 | 963.5 | A39 | 839.7 |
| A3 | 908.3 | A40 | 793.7 |
| A4 | 850.4 | A41 | 878.4 |
| A5 | 892.6 | A42 | 907.4 |
| A6 | 963.5 | A43 | 797.6 |
| A7 | 895.8 | A44 | 807.7 |
| A8 | 949.6 | A45 | 920.5 |
| A9 | 920.5 | A46 | 865.5 |
| A10 | 836.6 | A47 | 894.4 |
| A11 | 894.7 | A48 | 895.8 |
| A12 | 893.5 | A49 | 837.4 |
| A13 | 842.5 | A50 | 853.5 |
| A14 | 949.7 | A51 | 892.5 |
| A15 | 921.5 | A52 | 806.3 |
| A16 | 878.7 | A53 | 798.0 |
| A17 | 864.5 | A54 | 786.5 |
| A18 | 837.6 | A55 | 781.6 |
| A19 | 821.6 | A56 | 821.0 |
| A20 | 894.5 | A57 | 817.5 |
| A21 | 795.4 | A58 | 767.4 |
| A22 | 878.5 | A59 | 823.5 |
| A23 | 880.3 | A60 | 876.6 |
| A24 | 914.6 | A61 | 779.6 |
| A25 | 795.4 | A62 | 863.7 |
| A26 | 837.5 | A63 | 848.6 |
| A27 | 850.5 | A64 | 833.7 |
| A28 | 823.6 | A65 | 866.7 |
| A29 | 906.5 | A66 | 838.4 |
| A30 | 852.6 | A67 | 810.5 |
| A31 | 894.8 | A68 | 838.7 |
| A32 | 836.5 | A69 | 851.7 |
| A33 | | A70 | 823.5 |
| A34 | 906.0 | A71 | 786.5 |
| A35 | 970.7 | A72 | 842.5 |
| A36 | 964.5 | A73 | 864.5 |
| A37 | 971.2 | A74 | 852.5 |
| A75 | 797.6 | A170 | 870.5 |
| A76 | 796.4 | A171 | 879.5 |
| A77 | 822 | A172 | 811.5 |
| A78 | 848.5 | A173 | 871.2 |
| A79 | 904.8 | A174 | 837.4 |
| A80 | 946.5 | A175 | 874.5 |
| A81 | 838.5 | A176 | 807.5 |
| A82 | 853.5 | A177 | 773 |
| A83 | 850.45 | A178 | 787 |
| A84 | 864.5 | A179 | 787 |
| A85 | 864.5 | A180 | 784 |
| A86 | 822.6 | A181 | 784 |
| A87 | 822.3 | A182 | 722.9 |
| A88 | 836.3 | A183 | 722 |
| A89 | 839.6 | A184 | 762 |
| A90 | 837.6 | A185 | 872.18 |
| A91 | 837.5 | A186 | 745.7 |
| A92 | 811.5 | A187 | 829.9 |
| A93 | 811.5 | A188 | 829.9 |
| A94 | 837.5 | A189 | 759.6 |
| A95 | 935.6 | A190 | 775.9 |
| A96 | 919.6 | A191 | 808.7 |
| A97 | 926.5 | A192 | 770.8 |
| A98 | 905.5 | A193 | 802.7 |
| A99 | 912.3 | A194 | 789.8 |
| A100 | 864.5 | A195 | 796.7 |
| A101 | 852.5 | A196 | 744.7 |
| A102 | 795.4 | A197 | 798.9 |
| A103 | 772.3 | A198 | 840.9 |
| A104 | 781.4 | A199 | 753.9 |
| A105 | 891.5 | A200 | 758.9 |
| A106 | 898.5 | A201 | 984.4 |
| A107 | 848.5 | A202 | 934.4 |
| A108 | 855.5 | A203 | 941.5 |
| A109 | 878.8 | A204 | 950.4 |
| A110 | 885.6 | A205 | 857.3 |
| A111 | 894.6 | A206 | 890.4 |
| A112 | 947.7 | A207 | 791.7 |
| A113 | 954.7 | A208 | 793.6 |
| A114 | 963.6 | A209 | 867.5 |
| A115 | 892.4 | A210 | 858.5 |
| A116 | 889.5 | A211 | 922.6 |
| A117 | 936.5 | A212 | 798.4 |
| A118 | 841.4 | A213 | 867.7 |
| A119 | 834.8 | A214 | 797.5 |
| A120 | 921.5 | A215 | 946.5 |
| A121 | 852.8 | A216 | 904.8 |
| A122 | 865.8 | A217 | 862.6 |
| A123 | 907.8 | A218 | 835.5 |
| A124 | 851.8 | A219 | 849.6 |
| A125 | 838 | A220 | 931.4 |
| A126 | 862.5 | A221 | 911.3 |
| A127 | 864.8 | A222 | 853.2 |
| A128 | 864.8 | A223 | 835.5 |
| A129 | 850.8 | A224 | 821.6 |
| A130 | 906 | A225 | 748.8 |
| A131 | 865.8 | A226 | 913.8 |
| A132 | 838.9 | A227 | 894.0 |
| A133 | 877.9 | A228 | 877.9 |
| A134 | 879.8 | A229 | 897.8 |
| A135 | 961.6 | A230 | 879.9 |
| A136 | 815.5 | A231 | 893.9 |
| A137 | 801.5 | A232 | 852.9 |
| A138 | 802.4 | A233 | 950.6 |
| A139 | 850.5 | A234 | 917.3 |
| A140 | 862.6 | A235 | 897.3 |
| A141 | 811.4 | A236 | 780.8 |
| A142 | 793.3 | A237 | 919.4 |
| A143 | 856.2 | A238 | 842.4 |
| A144 | 793.5 | A239 | 826.4 |
| A145 | 836.2 | A240 | 851.8 |
| A146 | 835.4 | A241 | 851.7 |
| A147 | 835.3 | A242 | 878.9 |
| A148 | 876.6 | A243 | 864.8 |
| A149 | 862.6 | A244 | 883.5 |
| A150 | 865.5 | A245 | 828.4 |
| A151 | 890.3 | A246 | 821.4 |
| A152 | 786.2 | A247 | 912.8 |
| A153 | 819.5 | A248 | 893.6 |
| A154 | 857.2 | A249 | 888.8 |
| A155 | 862.6 | A250 | 899.8 |
| A156 | 847.5 | A251 | 864.7 |
| A157 | 849.5 | A252 | 905.8 |
| A158 | 849.5 | A253 | 750.7 |
| A159 | 846.6 | A254 | 787.8 |
| A160 | 839.6 | A255 | 851.6 |
| A161 | 839.5 | A256 | 795.4 |
| A162 | 839.5 | A257 | 852.6 |
| A163 | 862.6 | A258 | 766.8 |
| A164 | 862.7 | A259 | 864.5 |
| A165 | 839.5 | A260 | 853.4 |
| A166 | 857.5 | A261 | 773.8 |
| A167 | 857.5 | A262 | 878.7 |
| A168 | 836.5 | A263 | 780.8 |
| A169 | 880.3 | | |
| A264 | 758.4 | A293 | 898.7 |
| A265 | 757.3 | A294 | 912.7 |
| A266 | 772.4 | A295 | 882.3 |
| A267 | 728.4 | A296 | 912.3 |
| A268 | 882.4 | A297 | 921.3 |
| A270 | 744.3 | A298 | 883.2 |
| A271 | 871.2 | A299 | 871.3 |
| A272 | 898.6 | A300 | 898.5 |
| A273 | 910.5 | A301 | 869.3 |
| A274 | 882.3 | A302 | 893.5 |
| A275 | 885.5 | A303 | 924.4 |
| A276 | 885.5 | A304 | 841.2 |
| A277 | 913.6 | A305 | 841.5 |
| A278 | 885.6 | A306 | 914.5 |
| A279 | 885.4 | A307 | 896.5 |
| A280 | 910.6 | A308 | 896.4 |
| A281 | 884.3 | A309 | 871.3 |
| A282 | 882.2 | A310 | 871.4 |
| A283 | 898.5 | A311 | 896.5 |
| A284 | 882.5 | A312 | 883.5 |
| A285 | 896.2 | A313 | 896.6 |
| A286 | 713.1 | A314 | 882.6 |
| A287 | 835.3 | A315 | 729.3 |
| A288 | 925.4 | A316 | 906.5 |
| A289 | 885.0 | A317 | 827.4 |
| A290 | 941.3 | A318 | 898.5 |
| A291 | 898.3 | A319 | 898.3 |
| A292 | 898.7 | A320 | 733.2 |
| A321 | 771.4 | A354 | 844.6 |
| A322 | 893.2 | A355 | 850.5 |
| A323 | 837.5 | A356 | 855.5 |
| A324 | 807.3 | A357 | 905.4 |
| A325 | 922.5 | A358 | 843.5 |
| A326 | 882.5 | A359 | 715.2 |
| A327 | 882.5 | A360 | 715.2 |
| A328 | 924.4 | A361 | 731.3 |
| A329 | 896.3 | A362 | 717.3 |
| A330 | 911.1 | A363 | 855.5 |
| A331 | 729.4 | A364 | 866.5 |
| A332 | 857.4 | A365 | 908.6 |
| A333 | 857.5 | A366 | 736.1 |
| A334 | 857.2 | A367 | 699.1 |
| A335 | 871.5 | A368 | 714.1 |
| A336 | 829.5 | A369 | 713.3 |
| A337 | 856.5 | A370 | 947.6 |
| A338 | 912.2 | A371 | 961.4 |
| A339 | 857.5 | A372 | 857.5 |
| A340 | 771.4 | A373 | 857.5 |
| A341 | 870.5 | A374 | 857.5 |
| A342 | 975.3 | A375 | 856.5 |
| A343 | 842.5 | A376 | 857.5 |
| A344 | 871.7 | A377 | 865.6 |
| A345 | 808.5 | A378 | 947.3 |
| A346 | 837.5 | A379 | 975.6 |
| A347 | 837.5 | A380 | 961.3 |
| A348 | 963.5 | A381 | 850.6 |
| A349 | 855.5 | A382 | 852.2 |
| A350 | 843.5 | A383 | 905.1 |
| A351 | 843.5 | A384 | 849.5 |
| A352 | 855.5 | A385 | 961.6 |
| A353 | 841.5 | A386 | 949.3 |
| A387 | 871.5 | A421 | 892.9 |
| A388 | 819.3 | A422 | 951.3 |
| A389 | 813.2 | A423 | 1051.6 |
| A391 | 851.7 | A424 | 939.4 |
| A392 | 851.7 | A425 | 927.4 |
| A393 | 891.8 | A426 | 953.40 |
| A394 | 879.8 | A427 | 978.3 |
| A395 | 879.8 | A428 | 918.2 |
| A396 | 921.8 | A429 | 911.3 |
| A397 | 909.8 | A430 | 804.5 |
| A398 | 736.4 | A431 | 891.5 |
| A399 | 827.5 | A432 | 879.5 |
| A400 | 841.5 | A433 | 940.7 |
| A401 | 857.5 | A434 | 896.5 |
| A402 | 871.4 | A435 | 896.3 |
| A403 | 871.5 | A436 | 926.2 |
| A404 | 906.9 | A437 | 946.6 |
| A405 | 865.9 | A438 | 896.1 |
| A406 | 863.8 | A439 | 988.1 |
| A407 | 891.9 | A440 | 988.1 |
| A408 | 919.9 | A441 | 926.2 |
| A409 | 908 | A442 | 910.9 |
| A410 | 878.0 | A443 | 967.1 |
| A411 | 878.0 | A444 | 912.1 |
| A412 | 878.0 | A445 | 912.1 |
| A413 | 922.0 | A446 | 882.2 |
| A414 | 894.9 | A447 | 867.1 |
| A415 | 928.0 | A448 | 953.2 |
| A416 | 901.9 | A449 | 953.5 |
| A417 | 890.9 | A450 | 1017.2 |
| A418 | 867.9 | A451 | 912.2 |
| A419 | 879.9 | A452 | 895.2 |
| A420 | 866.9 | A453 | 924.2 |
| A454 | 844.2 | A482 | 960.1 |
| A455 | 901.9 | A483 | 1008.1 |
| A456 | 867.2 | A484 | 912.2 |
| A457 | 940.6 | A485 | 938.6 |
| A458 | 898.5 | A486 | 952.3 |
| A459 | 954.8 | A487 | 885.3 |
| A460 | 896.2 | A488 | 884.3 |
| A461 | 924.2 | A489 | 886.2 |
| A462 | 896.2 | A490 | 1017.9 |
| A463 | 856.2 | A491 | 912.6 |
| A464 | 931.2 | A492 | 912.6 |
| A465 | 981.7 | A493 | 912.6 |
| A466 | 955.3 | A494 | 912.6 |
| A467 | 940.6 | A495 | 924.2 |
| A468 | 910.6 | A496 | 917.0 |
| A469 | 884.5 | A497 | 882.1 |
| A470 | 896.2 | A498 | 924.6 |
| A471 | 896.1 | A499 | 912.1 |
| A472 | 912.1 | A500 | 921.2 |
| A473 | 898.6 | A501 | 984.1 |
| A474 | 899.2 | A502 | 884.6 |
| A475 | 899.1 | A503 | 896.1 |
| A476 | 996.3 | A504 | 898.1 |
| A477 | 968.6 | A505 | 954.7 |
| A478 | 885.5 | A506 | 902.1 |
| A479 | 910.9 | A507 | 1011.2 |
| A480 | 910.1 | A508 | 884.6 |
| A481 | 896.9 | A509 | 943.2 |
| A510 | 883.5 | A538 | 898.2 |
| A511 | 952.3 | A539 | 896.5 |
| A512 | 940.6 | A540 | 870.0 |
| A513 | 910.3 | A541 | 882.1 |
| A514 | 901.2 | A542 | 884.2 |
| A515 | 901.2 | A543 | 940.9 |
| A516 | 896.3 | A544 | 874.2 |
| A517 | 896.2 | A545 | 897.9 |
| A518 | 898.6 | A546 | 928.2 |
| A519 | 898.6 | A547 | 912.5 |
| A520 | 911.2 | A548 | 912.5 |
| A521 | 897.2 | A549 | 920.5 |
| A522 | 883.2 | A550 | 934.5 |
| A523 | 853.2 | A551 | 934.4 |
| A524 | 946.2 | A552 | 920.5 |
| A525 | 896.2 | A553 | 887.1 |
| A526 | 940.3 | A554 | 837.4 |
| A527 | 871.2 | A555 | 955.2 |
| A528 | 883.2 | A556 | 932.2 |
| A529 | 951.8 | A557 | 906.1 |
| A530 | 945.1 | A558 | 856.2 |
| A531 | 898.3 | A559 | 888.2 |
| A532 | 954.5 | A560 | 869.1 |
| A533 | 899.5 | A561 | 883.2 |
| A534 | 899.5 | A562 | 1009.3 |
| A535 | 884.2 | A563 | 884.6 |
| A536 | 939.5 | A564 | 884.6 |
| A537 | 939.5 | A565 | 924.2 |
| A566 | 884.9 | A592 | 961.2 |
| A567 | 874.2 | A593 | 1043.0 |
| A568 | 896.2 | A594 | 1025.8 |
| A569 | 898.2 | A595 | 967.5 |
| A570 | 870.2 | A596 | 967.5 |
| A571 | 899.0 | A597 | 900.1 |
| A572 | 914.2 | A598 | 845.4 |
| A573 | 912.2 | A599 | 917.2 |
| A574 | 913.9 | A600 | 933.1 |
| A575 | 914.2 | A601 | 898.0 |
| A576 | 885.0 | A602 | 897.0 |
| A577 | 1024.7 | A603 | 884.9 |
| A578 | 869.5 | A604 | 933.1 |
| A579 | 871.4 | A605 | 926.1 |
| A580 | 886.9 | A606 | 940.5 |
| A581 | 872.1 | A607 | 924.3 |
| A582 | 933.2 | A608 | 896.3 |
| A583 | 1016.2 | A609 | 898.4 |
| A584 | 927.2 | A610 | 898.5 |
| A585 | 918.2 | A611 | 870.5 |
| A586 | 911.3 | A612 | 858.4 |
| A587 | 899.4 | A613 | 858.3 |
| A588 | 898.6 | A614 | 899.4 |
| A589 | 910.2 | A615 | 926.4 |
| A590 | 914.6 | A616 | 926.4 |
| A591 | 915.4 | | |

Blank = not determined

### Biological Assays

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: this protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On day of assay, 40 nl of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μl lysis buffer, with shaking at 600 RPM at room temperature. Lysate (10 μl) was transferred to a 384-well Opti-plate (PerkinElmer) and 5 μl acceptor mix was added. After a 2-hour incubation in the dark, 5 μl donor mix was added, plate was sealed and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μl tris buffered saline (TBS) and fixed for 15 minutes with 150 μl 4% paraformaldehyde in TBS. Fixed cells were washed 4 times for 5 minutes with TBS containing 0.1% Triton X-100 (TBST) and then blocked with 100 μl Odyssey blocking buffer (LI-COR) for 60 minutes at room temperature. Primary antibody (PERK, CST-4370, Cell Signaling Technology) was diluted 1:200 in blocking buffer, and 50 μl was added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li—COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μl of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μl) were transferred to the next wells containing 30 μl of 100% DMSO, and repeated until a 9-concentration 3-fold serial dilution was made (starting assay concentration of 10 μM). Test compounds (132.5 nl) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μl) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC₅₀ values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} were combined in a 384-well assay plate at final concentrations of 25 μM, 12.5 nM and 50 nM, respectively. Compound was present in plate wells as a 10-point 3-fold dilution series starting at a final concentration of 30 μM. After incubation at 25° C. for 3 hours, a mixture of Anti-His Eu—W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras: RAF complex were identified as those eliciting a decrease in the TR-FRET ratio relative to DMSO control wells.

TABLE 4
Biological Assay Data for Representative Compounds of the Present Invention

| | FRET | FRET | FRET | FRET | FRET | H358 | Capan-1 | ASPC-1 | H358 Cell |
|---|---|---|---|---|---|---|---|---|---|
| | G12C | G12V | G12D | G13C | G13D | pERK | pERK | pERK | Viability |
| | IC50, | IC50, | IC50, | IC50, | IC50, | EC50, | EC50, | EC50, | IC50, |
| Ex# | uM | uM | uM | uM | uM | uM | uM | uM | uM |
| A74 | 0.45 | 3.67 | 0.228 | 0.091 | 0.212 | 0.334 | 0.413 | 0.468 | |
| A73 | 0.082 | 0.905 | 0.061 | 0.013 | 0.033 | 0.036 | 0.197 | 0.079 | |
| A3 | 0.029 | 0.043 | 0.545 | 0.099 | 0.16 | 0.018 | 0.008 | 0.144 | 0.057 |
| A25 | 0.128 | 0.197 | 1.29 | 0.097 | 0.235 | 0.064 | 0.052 | 0.706 | 0.226 |
| A12 | 0.068 | 0.329 | 0.154 | 0.148 | 0.219 | | | | 4.29 |

Blank = not determined
Additional H358 Cell Viability assay data
*Key:
+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 5
H358 Cell Viability assay data (K-Ras G12C, IC50, uM):

| IC50* | Examples |
|---|---|
| + | A136, A159, A205, A277, A278, A289, A291, A296, A298, |
| | A302, A303, A304, A306, A309, A310, A325, A335, A338, |
| | A356, A358, A365, A372, A373, A374, A382, A399, A439, |
| | A443, A450, A457, A465, A466, A476, A477, A478, A483, |
| | A484, A487, A490, A500, A501, A505, A514, A515, A526, |
| | A529, A536, A543, A546, A551, A555, A561, A562, A573, |
| | A577, A583, A590, A593, A594, A606, A607 |
| ++ | A114, A117, A13, A131, A138, A141, A147, A156, A160, |
| | A162, A164, A165, A170, A202, A204, A211, A217, A218, |
| | A223, A224, A233, A240, A242, A247, A248, A249, A250, |
| | A252, A273, A279, A280, A285, A286, A288, A290, A293, |
| | A294, A295, A297, A299, A3, A301, A305, A307, A311, |
| | A312, A313, A316, A318, A319, A326, A329, A330, A333, |
| | A334, A336, A337, A342, A348, A349, A350, A351, A352, |
| | A353, A357, A363, A364, A375, A376, A377, A379, A383, |
| | A387, A389, A391, A392, A400, A401, A402, A403, A406, |
| | A415, A422, A433, A436, A440, A441, A444, A445, A451, |
| | A452, A454, A458, A459, A467, A481, A485, A486, A488, |
| | A489, A491, A492, A493, A494, A495, A498, A499, A502, |
| | A503, A506, A507, A509, A510, A511, A512, A513, A518, |
| | A520, A521, A522, A523, A525, A527, A528, A531, A532, |
| | A533, A534, A537, A538, A540, A541, A542, A547, A548, |
| | A549, A550, A552, A553, A557, A559, A560, A563, A564, |
| | A565, A566, A567, A568, A569, A570, A571, A574, A576, |
| | A578, A582, A584, A585, A587, A588, A589, A591, A595, |
| | A596, A597, A601, A603, A605, A614, A615 |
| +++ | A100, A11, A116, A121, A123, A124, A126, A130, A132, |
| | A137, A139, A143, A146, A152, A155, A157, A161, A166, |
| | A167, A168, A169, A171, A173, A174, A18, A184, A19, |
| | A201, A203, A209, A21, A210, A219, A221, A226, A228, |
| | A231, A232, A238, A239, A241, A243, A245, A25, A252, |
| | A26, A260, A264, A266, A267, A268, A270, A274, A276, |
| | A28, A281, A282, A283, A284, A287, A29, A292, A30, |
| | A308, A314, A315, A317, A321, A322, A323, A332, A339, |
| | A343, A346, A35, A354, A355, A360, A361, A362, A367, |
| | A368, A369, A378, A381, A384, A385, A386, A39, A393, |
| | A395, A396, A397, A407, A408, A409, A410, A411, A412, |
| | A413, A414, A416, A417, A418, A420, A421, A423, A437, |
| | A438, A442, A447, A449, A455, A461, A462, A471, A480, |
| | A482, A496, A497, A5, A504, A508, A524, A535, A539, |
| | A54, A544, A545, A554, A556, A572, A575, A579, A580, |
| | A581, A586, A592, A598, A600, A602, A604, A608, A610, |
| | A611, A612, A613, A616, A64, A7, A78, A8, A90, A91, |
| | A94, A95 |
| ++++ | A1, A10, A101, A102, A104, A111, A117, A12, A120, |
| | A125, A127, A128, A129, A134, A148, A15, A16, A163, |
| | A17, A2, A20, A216, A22, A227, A23, A24, A244, A254, |
| | A254, A256, A258, A259, A261, A262, A27, A3, A320, |
| | A359, A36, A36, A37, A38, A4, A40, A405, A41, A42, |
| | A43, A44, A45, A453, A46, A464, A50, A51, A517, A519, |
| | A52, A53, A54, A55, A57, A58, A59, A599, A6, A60, |
| | A609, A61, A65, A66, A67, A70, A82, A83, A85, A9, A92, |
| | A97 |
| +++++ | A12, A133, A14, A31, A32, A4, A47, A48, A56, A62, A63, |
| | A68, A69, A84, A99 |

*Key:
+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 6
Capan-1 Cell Viability assay data (K-Ras G12V, IC50, uM):

| IC50* | Examples |
|---|---|
| + | A277, A450, A465, A466, A476, A477, A484, A500, A505, A526, A529, A555, A562, A577, |
| | A583, A593, A594 |
| ++ | A114, A117, A132, A136, A138, A141, A156, A159, A162, A165, A170, A202, A204, A205, |
| | A210, A211, A218, A224, A233, A240, A247, A250, A278, A279, A280, A285, A288, A289, A290, |
| | A291, A293, A295, A296, A298, A3, A302, A303, A304, A306, A309, A310, A312, A313, A316, |
| | A318, A319, A325, A329, A330, A334, A335, A336, A338, A353, A356, A357, A358, A363, A364, |
| | A365, A372, A373, A374, A376, A377, A382, A383, A387, A389, A399, A400, A401, A402, |
| | A403, A415, A433, A436, A439, A440, A443, A444, A445, A451, A452, A454, A457, A458, A467, |
| | A472, A474, A475, A478, A481, A483, A485, A486, A487, A490, A491, A494, A499, A503, A506, |
| | A509, A510, A511, A512, A513, A514, A515, A518, A520, A521, A523, A525, A527, A528, A531, |
| | A532, A533, A534, A536, A537, A538, A540, A543, A546, A547, A548, A549, A550, A551, |
| | A561, A563, A565, A566, A569, A570, A571, A573, A574, A576, A587, A590, A591, A601, A603, |
| | A606, A607, A608, A614, A615 |
| +++ | A102, A11, A116, A121, A123, A126, A13, A131, A137, A139, A143, A146, A147, A152, A157, |
| | A160, A161, A164, A166, A167, A168, A169, A171, A173, A174, A18, A19, A201, A203, A209, |
| | A21, A217, A219, A221, A223, A226, A228, A232, A238, A239, A241, A242, A244, A245, A248, |
| | A249, A25, A252, A252, A254, A26, A264, A266, A267, A268, A273, A274, A275, A276, A281, |
| | A282, A283, A284, A286, A287, A292, A294, A297, A299, A30, A301, A305, A307, A308, A311, |
| | A314, A315, A320, A321, A322, A323, A326, A332, A333, A337, A342, A343, A346, A347, A348, |
| | A349, A350, A351, A352, A354, A355, A360, A361, A362, A367, A368, A369, A375, A379, |
| | A384, A385, A386, A39, A395, A396, A397, A406, A407, A409, A410, A411, A412, A413, A416, |
| | A419, A420, A422, A423, A437, A441, A447, A449, A455, A459, A461, A462, A468, A469, |
| | A470, A471, A473, A480, A482, A488, A489, A492, A493, A495, A496, A497, A498, A5, A501, |
| | A502, A504, A507, A508, A516, A517, A522, A524, A530, A535, A539, A54, A541, A542, A544, |
| | A545, A552, A553, A556, A557, A558, A559, A560, A564, A567, A568, A575, A578, A579, A580, |
| | A582, A584, A585, A586, A588, A589, A595, A596, A597, A598, A600, A602, A605, A609, |
| | A610, A611, A612, A613, A616, A90, A91, A94, A95 |
| ++++ | A1, A100, A101, A106, A107, A111, A112, A113, A115, A124, A125, A129, A130, A135, A144, |
| | A148, A149, A155, A158, A16, A17, A175, A176, A179, A180, A182, A183, A184, A185, A200, |
| | A216, A220, A225, A225, A229, A230, A231, A235, A236, A237, A24, A246, A253, A254, A259, |
| | A260, A261, A262, A265, A270, A272, A28, A29, A300, A317, A324, A327, A331, A339, A345, |
| | A359, A366, A370, A371, A378, A380, A381, A388, A391, A392, A393, A394, A398, A40, A405, |
| | A408, A414, A417, A418, A421, A43, A434, A435, A438, A442, A453, A456, A460, A463, A464, |
| | A479, A519, A54, A554, A572, A581, A592, A599, A604, A61, A7, A76, A78, A8, A80, A82, |
| | A83, A85, A89, A93, A96, A97 |
| +++++ | A104, A104, A105, A108, A109, A110, A116, A117, A118, A119, A120, A122, A127, A128, |
| | A133, A134, A140, A142, A145, A150, A151, A153, A154, A163, A172, A177, A178, A178, A179, |
| | A181, A186, A187, A188, A189, A190, A191, A192, A193, A194, A195, A196, A197, A198, A199, |
| | A206, A207, A208, A212, A213, A214, A215, A222, A227, A227, A234, A236, A243, A251, |
| | A253, A255, A258, A271, A328, A340, A341, A344, A4, A404, A446, A448, A75, A79, A84, A86, |
| | A87, A87, A88, A92, A98, A99 |

Additional Ras-Raf disruption/FRET/MOA assay data (IC50, uM):
*Key:
+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 7
KRAS G12D FRET data

| IC50* | Examples |
|---|---|
| + | None |
| ++ | A1, A100, A111, A120, A124, A125, A127, A128, A129, A131, A133, A134, A135, A139, A140, |
| | A148, A159, A164, A223, A227, A228, A231, A242, A243, A247, A249, A325, A342, A348, A365, |
| | A370, A371, A378, A379, A380, A381, A385, A386, A391, A392, A393, A395, A397, A4, A415, |
| | A419, A427, A483, A494, A501, A507, A546, A573, A577, A584, A594, A605, A95 |
| +++ | A10, A101, A102, A106, A114, A12, A121, A122, A123, A126, A130, A132, A136, A14, A146, |
| | A147, A149, A15, A151, A155, A156, A157, A158, A160, A161, A162, A163, A165, A166, A167, |
| | A168, A169, A171, A174, A2, A201, A202, A204, A205, A209, A211, A216, A217, A218, A219, |
| | A224, A227, A229, A23, A230, A232, A233, A240, A241, A248, A248, A250, A251, A252, A252, |
| | A255, A259, A264, A265, A266, A267, A268, A27, A270, A273, A274, A275, A277, A278, A279, |
| | A280, A285, A286, A287, A288, A289, A290, A291, A294, A298, A3, A302, A303, A304, A306, |
| | A309, A31, A310, A311, A312, A313, A314, A32, A321, A323, A332, A333, A334, A335, A336, |
| | A343, A346, A347, A349, A350, A351, A353, A356, A358, A363, A364, A372, A373, A374, A376, |
| | A377, A382, A383, A384, A394, A396, A399, A400, A401, A402, A404, A405, A406, A407, |
| | A408, A409, A41, A410, A411, A412, A413, A414, A416, A417, A418, A420, A421, A422, A423, |
| | A424, A426, A432, A434, A435, A436, A438, A441, A443, A444, A447, A45, A450, A454, |
| | A457, A458, A459, A463, A465, A466, A467, A468, A469, A471, A475, A476, A477, A478, A48, |
| | A484, A485, A487, A488, A491, A492, A493, A498, A5, A500, A502, A503, A505, A506, A509, |
| | A514, A515, A518, A520, A523, A526, A528, A529, A531, A533, A534, A536, A537, A538, A542, |
| | A543, A545, A549, A551, A552, A554, A555, A557, A558, A559, A560, A561, A562, A563, |
| | A564, A565, A566, A567, A568, A569, A571, A574, A576, A578, A580, A581, A582, A583, A586, |
| | A587, A588, A589, A590, A591, A593, A595, A596, A6, A600, A601, A603, A606, A607, A608, |
| | A610, A611, A614, A615, A616, A62, A66, A67, A68, A7, A78, A79, A8, A80, A81, A83, A85, A87, |
| | A87, A88, A89, A99 |
| ++++ | A105, A107, A108, A109, A11, A110, A112, A113, A117, A118, A119, A12, A13, A137, A138, |
| | A144, A152, A16, A17, A170, A173, A175, A176, A177, A178, A179, A179, A18, A184, A19, A20, |
| | A208, A21, A210, A213, A215, A22, A222, A225, A226, A236, A239, A24, A25, A253, A254, |
| | A254, A257, A26, A260, A262, A272, A276, A28, A282, A283, A284, A292, A293, A295, A296, |
| | A297, A299, A30, A300, A301, A305, A307, A308, A315, A316, A318, A319, A320, A322, A324, |
| | A326, A329, A33, A330, A331, A337, A338, A339, A344, A345, A35, A352, A354, A355, A357, |
| | A359, A36, A36, A360, A361, A362, A366, A367, A368, A369, A375, A38, A387, A389, A39, A4, |
| | A40, A403, A425, A428, A43, A431, A433, A437, A439, A44, A440, A442, A445, A448, A449, |
| | A451, A452, A455, A456, A46, A460, A461, A462, A464, A47, A470, A472, A473, A474, A479, |
| | A480, A481, A486, A489, A49, A490, A495, A496, A497, A499, A50, A504, A508, A51, A510, |
| | A511, A512, A513, A516, A517, A519, A521, A522, A524, A525, A527, A530, A532, A535, A539, |
| | A540, A541, A544, A547, A548, A550, A553, A556, A570, A572, A575, A579, A585, A592, A597, |
| | A598, A599, A602, A604, A609, A612, A64, A65, A69, A70, A76, A82, A84, A9, A90, A91, A92, |
| | A93, A94, A96, A97, A98, A613 |
| +++++ | A103, A103, A104, A104, A115, A116, A116, A117, A141, A142, A143, A145, A150, A153, A154, |
| | A172, A178, A180, A181, A182, A183, A185, A186, A187, A188, A189, A190, A191, A192, |
| | A193, A194, A195, A196, A197, A198, A199, A200, A203, A206, A207, A212, A214, A220, A221, |
| | A225, A234, A235, A236, A237, A238, A244, A245, A246, A253, A256, A258, A261, A271, A281, |
| | A29, A3, A317, A327, A328, A340, A341, A37, A388, A398, A42, A429, A430, A446, A453, |
| | A482, A52, A53, A54, A54, A55, A56, A57, A58, A59, A60, A61, A63, A75, A77, A86 |

TABLE 8
KRAS G12C FRET data

| IC50* | Examples |
|---|---|
| + | A323, A325, A347, A501, A546, A577, A594 |
| ++ | A1, A10, A100, A11, A111, A114, A117, A12, A120, A121, A125, A126, A127, A128, A129, A13, |
| | A131, A132, A135, A136, A139, A14, A140, A146, A147, A148, A149, A15, A151, A155, A156, |
| | A157, A159, A16, A160, A162, A164, A165, A166, A168, A17, A18, A19, A2, A20, A201, A202, |
| | A204, A205, A211, A216, A217, A218, A219, A223, A224, A226, A227, A228, A229, A230, A231, |
| | A233, A240, A241, A242, A243, A247, A248, A248, A249, A250, A252, A252, A255, A262, |
| | A264, A265, A266, A273, A274, A275, A277, A278, A279, A280, A285, A288, A289, A290, A291, |
| | A298, A3, A302, A303, A304, A306, A309, A310, A312, A316, A321, A330, A333, A334, A335, |
| | A336, A338, A342, A343, A346, A348, A349, A350, A351, A353, A356, A358, A363, A364, A365, |
| | A370, A371, A372, A373, A374, A376, A377, A378, A379, A380, A381, A382, A383, A384, |
| | A385, A386, A387, A391, A392, A393, A395, A396, A397, A399, A4, A400, A401, A402, A405, |
| | A406, A407, A408, A409, A410, A411, A412, A413, A414, A415, A418, A419, A420, A422, A424, |
| | A426, A427, A432, A438, A443, A444, A450, A452, A454, A457, A458, A459, A465, A466, A467, |
| | A471, A475, A477, A478, A483, A484, A487, A488, A489, A491, A493, A494, A498, A5, A500, |
| | A503, A505, A507, A509, A510, A514, A515, A523, A526, A528, A529, A533, A534, A536, |
| | A537, A538, A540, A543, A549, A550, A551, A552, A554, A555, A557, A558, A560, A561, A562, |
| | A565, A567, A569, A571, A573, A574, A576, A578, A581, A582, A583, A584, A586, A590, |
| | A591, A593, A595, A596, A598, A6, A600, A601, A605, A606, A607, A608, A614, A615, A616, |
| | A62, A67, A68, A7, A8, A87, A9, A95 |
| +++ | A101, A102, A105, A106, A12, A122, A123, A124, A130, A133, A134, A137, A138, A144, A158, |
| | A161, A163, A167, A169, A170, A171, A173, A174, A176, A178, A179, A179, A182, A183, A184, |
| | A207, A208, A209, A21, A210, A215, A22, A225, A227, A23, A232, A236, A24, A25, A251, |
| | A253, A254, A254, A257, A259, A26, A260, A267, A268, A27, A270, A276, A28, A282, A283, |
| | A284, A286, A287, A29, A292, A293, A294, A295, A296, A297, A299, A30, A301, A305, A308, A31, |
| | A311, A313, A314, A315, A318, A319, A32, A320, A322, A324, A326, A329, A33, A331, A332, |
| | A337, A339, A34, A344, A345, A35, A352, A354, A355, A357, A359, A36, A36, A360, A361, |
| | A362, A366, A367, A368, A369, A37, A375, A38, A389, A39, A394, A4, A40, A403, A404, A41, |
| | A416, A417, A42, A421, A423, A425, A43, A431, A433, A434, A435, A436, A437, A439, A44, A440, |
| | A441, A445, A447, A449, A45, A451, A453, A455, A456, A46, A460, A461, A462, A463, A468, |
| | A469, A47, A472, A473, A474, A476, A479, A48, A480, A481, A485, A486, A49, A490, A492, |
| | A495, A496, A497, A499, A50, A502, A504, A506, A508, A51, A511, A512, A513, A518, A519, |
| | A52, A520, A521, A522, A524, A525, A527, A53, A530, A531, A532, A535, A541, A542, A544, |
| | A545, A547, A548, A553, A556, A559, A563, A564, A566, A568, A570, A579, A580, A585, A587, |
| | A588, A589, A592, A597, A602, A603, A610, A611, A612, A64, A65, A66, A69, A76, A78, A79, |
| | A80, A81, A82, A83, A84, A85, A86, A87, A88, A89, A90, A91, A93, A94, A96, A97, A98, A99 |
| ++++ | A104, A107, A108, A109, A110, A112, A113, A115, A116, A117, A118, A119, A141, A142, A143, |
| | A150, A152, A175, A177, A178, A180, A181, A185, A199, A203, A206, A212, A213, A214, |
| | A220, A221, A222, A225, A236, A237, A238, A239, A244, A245, A246, A253, A256, A258, A261, |
| | A271, A272, A281, A3, A300, A307, A317, A388, A398, A428, A429, A442, A446, A448, A464, |
| | A470, A482, A516, A517, A539, A54, A54, A55, A56, A57, A572, A575, A58, A59, A599, A60, |
| | A604, A609, A61, A63, A70, A75, A77, A92, A613 |
| +++++ | A103, A103, A104, A116, A145, A153, A154, A172, A186, A187, A188, A189, A190, A191, A192, |
| | A193, A194, A195, A196, A197, A198, A200, A234, A235, A327, A328, A340, A341, A430 |

TABLE 9
KRAS G12S FRET data

| IC50* | Examples |
|---|---|
| + | A501, A577, A594 |
| ++ | A1, A10, A100, A111, A114, A120, A121, A124, A125, A127, A128, A129, A131, A135, A139, |
| | A140, A147, A148, A156, A159, A162, A164, A165, A2, A202, A204, A211, A217, A218, A219, |
| | A223, A224, A227, A228, A230, A242, A243, A247, A248, A248, A249, A250, A252, A252, A273, |
| | A275, A277, A291, A3, A312, A323, A325, A335, A342, A347, A348, A349, A351, A363, A365, |
| | A370, A371, A377, A378, A379, A380, A381, A385, A386, A391, A392, A393, A395, A396, A397, |
| | A4, A400, A405, A406, A407, A408, A409, A411, A414, A415, A418, A419, A422, A424, A427, |
| | A459, A465, A483, A491, A494, A498, A5, A500, A503, A507, A526, A529, A537, A546, A554, |
| | A555, A558, A561, A565, A573, A578, A584, A590, A6, A605, A606, A607, A615, A68, A7, A87, |
| | A95 |
| +++ | A101, A102, A11, A117, A119, A12, A122, A123, A126, A13, A130, A132, A133, A134, A136, A14, |
| | A146, A149, A15, A151, A155, A157, A158, A16, A160, A161, A163, A166, A167, A168, A169, |
| | A17, A170, A171, A173, A174, A18, A184, A19, A20, A201, A205, A209, A21, A215, A216, |
| | A22, A226, A227, A229, A23, A231, A232, A233, A236, A24, A240, A241, A25, A251, A254, A254, |
| | A255, A257, A259, A26, A260, A262, A264, A265, A266, A267, A268, A27, A270, A274, A276, |
| | A278, A279, A28, A280, A284, A285, A286, A287, A288, A289, A29, A290, A293, A294, A295, |
| | A296, A297, A298, A30, A301, A302, A303, A304, A306, A309, A31, A310, A311, A313, A314, |
| | A315, A316, A318, A319, A32, A320, A321, A324, A326, A329, A33, A330, A331, A332, A333, |
| | A334, A336, A337, A338, A343, A344, A346, A35, A350, A352, A353, A354, A356, A357, |
| | A358, A36, A360, A361, A362, A364, A367, A368, A369, A37, A372, A373, A374, A375, A376, |
| | A38, A382, A383, A384, A387, A39, A394, A399, A40, A401, A402, A403, A404, A41, A410, A412, |
| | A413, A416, A417, A420, A421, A423, A426, A43, A431, A432, A433, A434, A435, A436, A437, |
| | A438, A441, A443, A444, A445, A447, A45, A450, A451, A452, A454, A455, A456, A457, A458, |
| | A46, A463, A466, A467, A468, A469, A47, A471, A472, A473, A474, A475, A476, A477, A478, |
| | A48, A480, A484, A485, A486, A487, A488, A489, A490, A492, A493, A495, A502, A505, |
| | A506, A508, A509, A51, A510, A511, A512, A513, A514, A515, A518, A520, A521, A522, A523, |
| | A525, A527, A528, A530, A531, A532, A533, A534, A535, A536, A538, A540, A541, A542, A543, |
| | A545, A547, A549, A550, A551, A552, A556, A557, A559, A560, A562, A563, A564, A566, A567, |
| | A568, A569, A570, A571, A574, A576, A580, A581, A582, A583, A585, A586, A587, A588, |
| | A589, A591, A593, A595, A596, A597, A598, A600, A601, A602, A603, A608, A610, A611, A614, |
| | A616, A62, A64, A65, A66, A67, A69, A76, A78, A79, A8, A80, A81, A83, A84, A85, A87, A88, |
| | A89, A9, A90, A91, A93, A94 |
| ++++ | A105, A106, A107, A108, A109, A110, A112, A113, A115, A116, A118, A12, A137, A138, A141, |
| | A142, A143, A144, A152, A175, A176, A177, A178, A178, A179, A179, A180, A181, A182, A183, |
| | A185, A203, A207, A208, A210, A213, A214, A220, A221, A222, A225, A225, A236, A237, |
| | A238, A239, A244, A245, A246, A253, A256, A258, A261, A271, A272, A281, A282, A283, A292, |
| | A299, A3, A300, A305, A307, A308, A317, A322, A339, A34, A345, A355, A359, A36, A366, |
| | A388, A389, A398, A4, A42, A425, A428, A439, A44, A440, A442, A446, A448, A449, A453, A460, |
| | A461, A462, A464, A470, A479, A481, A482, A49, A496, A497, A499, A50, A504, A516, A517, |
| | A519, A52, A524, A53, A539, A54, A54, A544, A548, A55, A553, A56, A57, A572, A575, A579, |
| | A58, A59, A592, A599, A60, A604, A609, A61, A612, A63, A70, A75, A82, A86, A92, A96, A97, |
| | A98, A99, A613 |
| +++++ | A103, A103, A104, A104, A116, A117, A145, A150, A153, A154, A172, A186, A187, A188, A189, |
| | A190, A191, A192, A193, A194, A195, A196, A197, A198, A199, A200, A206, A212, A234, |
| | A235, A253, A327, A328, A340, A341, A429, A430, A77 |

TABLE 10
KRAS G13C FRET data

| IC50* | Examples |
|---|---|
| + | A381, A325, A501, A594 |
| ++ | A1, A10, A100, A101, A102, A111, A114, A121, A123, A124, A125, A126, A127, A128, A129, |
| | A130, A131, A132, A133, A134, A135, A139, A140, A146, A147, A148, A149, A151, A155, A156, |
| | A159, A160, A162, A164, A165, A166, A168, A169, A171, A184, A201, A202, A204, A21, A211, |
| | A215, A216, A217, A218, A219, A223, A224, A226, A227, A227, A228, A229, A23, A230, A231, |
| | A233, A240, A241, A242, A243, A247, A248, A248, A249, A25, A250, A251, A252, A252, |
| | A255, A266, A27, A275, A277, A3, A31, A323, A324, A342, A346, A347, A348, A349, A351, A364, |
| | A365, A370, A371, A377, A378, A379, A380, A384, A385, A386, A391, A392, A393, A394, |
| | A395, A396, A397, A4, A405, A406, A407, A408, A409, A41, A410, A413, A414, A415, A418, |
| | A419, A420, A421, A422, A424, A426, A427, A432, A459, A465, A500, A507, A509, A526, A529, |
| | A546, A554, A555, A562, A573, A577, A578, A584, A605, A607, A615, A616, A67, A68, A78, |
| | A87, A88, A89, A95 |
| +++ | A105, A106, A107, A109, A11, A117, A118, A119, A12, A120, A122, A13, A136, A138, A14, A142, |
| | A144, A15, A157, A158, A16, A161, A163, A167, A17, A170, A174, A175, A176, A177, A178, |
| | A178, A179, A179, A18, A180, A181, A182, A183, A185, A19, A2, A20, A205, A207, A208, |
| | A209, A214, A22, A225, A225, A232, A236, A236, A24, A253, A254, A254, A256, A257, A258, |
| | A259, A26, A262, A264, A265, A267, A268, A270, A273, A274, A276, A278, A279, A28, A280, |
| | A284, A285, A286, A287, A288, A289, A290, A291, A293, A294, A295, A296, A297, A298, A30, |
| | A302, A303, A304, A306, A309, A310, A311, A312, A313, A314, A315, A316, A318, A319, A32, |
| | A320, A321, A322, A329, A33, A330, A331, A332, A333, A334, A335, A336, A337, A338, A343, |
| | A344, A345, A35, A350, A352, A353, A354, A355, A356, A357, A358, A359, A360, A361, A362, |
| | A363, A366, A367, A368, A369, A37, A372, A373, A374, A375, A376, A382, A383, A387, |
| | A388, A39, A398, A399, A4, A40, A400, A401, A402, A403, A404, A411, A412, A416, A417, A423, |
| | A425, A43, A431, A433, A434, A435, A436, A437, A438, A439, A441, A443, A444, A445, A447, |
| | A449, A45, A450, A451, A452, A454, A456, A457, A458, A46, A462, A463, A466, A467, A468, |
| | A469, A47, A470, A471, A472, A473, A474, A475, A476, A477, A478, A48, A480, A483, A484, |
| | A485, A486, A487, A488, A489, A490, A491, A492, A493, A494, A495, A497, A498, A5, A502, |
| | A503, A504, A505, A506, A508, A51, A510, A511, A512, A513, A514, A515, A518, A520, A521, |
| | A522, A523, A524, A525, A528, A530, A531, A532, A533, A534, A535, A536, A537, A538, A540, |
| | A541, A542, A543, A544, A545, A547, A548, A549, A55, A550, A551, A552, A553, A556, |
| | A557, A558, A559, A560, A561, A563, A564, A565, A566, A567, A568, A569, A570, A571, A574, |
| | A576, A579, A580, A581, A582, A583, A585, A586, A587, A588, A589, A590, A591, A592, A593, |
| | A595, A596, A597, A598, A6, A600, A601, A602, A603, A606, A608, A610, A611, A614, A62, |
| | A65, A66, A69, A7, A75, A76, A79, A8, A80, A81, A82, A83, A84, A85, A86, A87, A9, A90, A91, |
| | A92, A93, A94, A96, A98, A99 |
| ++++ | A104, A104, A108, A110, A112, A113, A115, A116, A12, A137, A141, A143, A150, A152, A172, |
| | A173, A188, A199, A203, A206, A210, A212, A213, A220, A221, A222, A234, A235, A237, A238, |
| | A239, A244, A245, A246, A253, A260, A261, A271, A272, A281, A282, A283, A29, A292, A299, |
| | A3, A300, A301, A305, A307, A308, A317, A326, A339, A34, A36, A36, A38, A389, A42, A428, |
| | A44, A440, A442, A446, A448, A453, A455, A460, A461, A464, A479, A481, A482, A49, A496, |
| | A499, A50, A516, A517, A519, A52, A527, A53, A539, A54, A54, A56, A57, A572, A575, A58, |
| | A599, A604, A609, A61, A612, A63, A64, A70, A77, A97, A613 |
| +++++ | A103, A103, A116, A117, A145, A153, A154, A186, A187, A189, A190, A191, A192, A193, A194, |
| | A195, A196, A197, A198, A200, A327, A328, A340, A341, A429, A430, A59, A60 |

TABLE 11
KRAS G12V FRET data

| IC50* | Examples |
|---|---|
| + | A325 |
| ++ | A1, A11, A114, A117, A121, A135, A139, A140, A146, A147, A156, A159, A160, A162, A164, |
| | A165, A2, A201, A202, A204, A211, A218, A219, A223, A224, A230, A233, A247, A248, A249, |
| | A250, A252, A264, A265, A266, A275, A277, A278, A279, A3, A323, A342, A347, A348, A349, |
| | A365, A370, A371, A377, A378, A379, A380, A385, A391, A396, A399, A405, A407, A415, A422, |
| | A423, A424, A427, A454, A465, A477, A487, A5, A500, A501, A507, A526, A529, A546, A554, |
| | A555, A562, A577, A578, A584, A594, A605, A607, A615, A95 |
| +++ | A10, A100, A102, A12, A120, A123, A124, A125, A126, A127, A128, A129, A13, A131, A132, |
| | A136, A137, A138, A14, A148, A149, A15, A151, A155, A157, A158, A16, A161, A166, A167, |
| | A168, A17, A170, A171, A174, A176, A18, A184, A19, A20, A205, A209, A21, A215, A217, A22, |
| | A226, A227, A228, A229, A231, A232, A236, A24, A240, A241, A242, A243, A248, A25, A251, |
| | A252, A254, A254, A255, A257, A26, A262, A267, A268, A270, A273, A274, A28, A280, A285, |
| | A286, A287, A288, A289, A29, A290, A291, A293, A294, A295, A296, A297, A298, A30, A301, |
| | A302, A303, A304, A306, A309, A310, A311, A312, A313, A314, A315, A316, A318, A319, A320, |
| | A321, A322, A324, A326, A329, A33, A330, A331, A332, A333, A334, A335, A336, A337, A338, |
| | A343, A344, A345, A346, A35, A350, A351, A352, A353, A354, A355, A356, A357, A358, A359, |
| | A36, A36, A360, A361, A362, A363, A364, A366, A367, A368, A369, A37, A372, A373, A374, |
| | A375, A376, A38, A381, A382, A383, A384, A386, A387, A39, A392, A393, A394, A395, A397, |
| | A4, A40, A400, A401, A402, A403, A406, A408, A409, A410, A411, A412, A413, A414, A416, |
| | A418, A419, A420, A425, A426, A43, A431, A433, A436, A437, A438, A44, A441, A443, A444, |
| | A445, A450, A451, A452, A456, A457, A458, A459, A463, A466, A467, A471, A472, A474, A475, |
| | A476, A478, A480, A483, A484, A485, A486, A488, A489, A49, A490, A491, A492, A493, A494, |
| | A495, A496, A498, A503, A505, A506, A509, A510, A511, A512, A513, A514, A515, A520, |
| | A521, A522, A523, A525, A528, A530, A531, A532, A533, A534, A535, A536, A537, A538, A540, |
| | A541, A543, A545, A547, A548, A549, A550, A551, A552, A553, A557, A558, A559, A560, |
| | A561, A564, A565, A566, A567, A568, A569, A570, A571, A573, A574, A576, A579, A580, A581, |
| | A582, A583, A585, A586, A589, A590, A591, A593, A595, A596, A597, A598, A6, A600, A601, |
| | A602, A603, A606, A608, A610, A614, A616, A62, A64, A67, A68, A7, A76, A78, A8, A83, A87, |
| | A89, A9, A90, A91, A93, A94 |
| ++++ | A101, A105, A106, A111, A112, A113, A115, A116, A117, A12, A122, A130, A133, A134, A141, |
| | A142, A143, A144, A152, A163, A169, A173, A177, A178, A178, A179, A179, A180, A181, A182, |
| | A183, A185, A199, A203, A207, A208, A210, A212, A214, A216, A220, A221, A225, A225, |
| | A227, A23, A236, A237, A238, A239, A244, A245, A246, A253, A253, A256, A258, A259, A260, |
| | A261, A27, A271, A276, A281, A282, A283, A284, A292, A299, A3, A300, A305, A307, A308, |
| | A31, A317, A32, A339, A34, A388, A389, A398, A404, A41, A417, A42, A421, A428, A432, A434, |
| | A435, A439, A440, A442, A447, A449, A453, A455, A46, A460, A461, A462, A464, A468, A469, |
| | A470, A473, A479, A48, A481, A482, A497, A499, A50, A502, A504, A508, A51, A516, A517, |
| | A518, A519, A52, A524, A527, A53, A539, A54, A54, A542, A544, A55, A556, A56, A563, A57, |
| | A572, A575, A58, A587, A588, A59, A592, A599, A60, A604, A609, A61, A611, A612, A63, A65, |
| | A66, A69, A75, A79, A80, A81, A82, A84, A85, A86, A87, A88, A92, A96, A97, A98, A99, A613 |
| +++++ | A103, A103, A104, A104, A107, A108, A109, A110, A116, A118, A119, A145, A150, A153, A154, |
| | A172, A175, A186, A187, A188, A189, A190, A191, A192, A193, A194, A195, A196, A197, |
| | A198, A200, A206, A213, A222, A234, A235, A272, A327, A328, A340, A341, A4, A429, A430, |
| | A446, A448, A45, A47, A70, A77 |

TABLE 12
KRAS WT FRET data

| IC50* | Examples |
|---|---|
| + | A594 |
| ++ | A1, A10, A100, A111, A114, A121, A124, A125, A126, A127, A128, A129, A130, A131, A135, |
| | A139, A140, A146, A147, A148, A149, A151, A155, A156, A157, A159, A160, A162, A164, A165, |
| | A166, A168, A2, A202, A204, A211, A216, A217, A218, A219, A223, A224, A227, A228, A23, |
| | A230, A231, A241, A242, A243, A247, A248, A249, A252, A252, A274, A275, A277, A278, A287, |
| | A290, A291, A298, A3, A306, A312, A323, A325, A333, A335, A336, A342, A346, A347, A348, |
| | A349, A351, A363, A365, A370, A371, A372, A373, A374, A377, A378, A379, A380, A381, A385, |
| | A386, A391, A392, A393, A395, A396, A397, A4, A400, A402, A405, A406, A407, A409, A410, |
| | A411, A412, A413, A415, A418, A419, A422, A424, A426, A427, A443, A454, A459, A465, A475, |
| | A483, A487, A491, A493, A494, A498, A500, A501, A503, A505, A507, A526, A528, A529, |
| | A536, A537, A545, A546, A554, A555, A558, A560, A561, A562, A565, A571, A573, A574, A577, |
| | A578, A584, A590, A596, A600, A605, A606, A607, A615, A616, A87, A95 |
| +++ | A101, A102, A106, A11, A117, A12, A120, A122, A123, A13, A132, A133, A134, A136, A137, |
| | A138, A14, A144, A15, A158, A16, A161, A163, A167, A169, A17, A170, A171, A173, A174, A176, |
| | A178, A179, A179, A18, A184, A19, A20, A201, A205, A208, A209, A21, A210, A215, A22, A226, |
| | A227, A229, A232, A233, A236, A24, A240, A248, A25, A250, A251, A254, A254, A255, A257, |
| | A259, A26, A260, A262, A264, A265, A266, A267, A268, A27, A270, A273, A276, A279, A28, |
| | A280, A283, A284, A285, A286, A288, A289, A292, A293, A294, A295, A296, A297, A299, A30, |
| | A301, A302, A303, A304, A308, A309, A31, A310, A311, A313, A314, A315, A316, A318, A319, |
| | A32, A320, A321, A322, A324, A326, A329, A330, A331, A332, A334, A337, A338, A339, |
| | A343, A344, A345, A35, A350, A352, A353, A354, A355, A356, A357, A358, A360, A361, A362, |
| | A364, A367, A368, A369, A37, A375, A376, A38, A382, A383, A384, A387, A39, A394, A399, |
| | A40, A401, A403, A404, A408, A41, A414, A416, A417, A420, A421, A423, A43, A432, A433, |
| | A434, A435, A436, A437, A438, A439, A44, A440, A441, A444, A445, A447, A449, A45, A450, |
| | A451, A452, A455, A456, A457, A458, A46, A461, A463, A466, A467, A468, A469, A47, A471, |
| | A472, A473, A474, A476, A477, A478, A48, A480, A484, A485, A486, A488, A489, A49, A490, |
| | A492, A495, A496, A5, A50, A502, A504, A506, A508, A509, A51, A510, A511, A512, A513, A514, |
| | A515, A518, A519, A520, A521, A522, A523, A524, A525, A527, A530, A531, A532, A533, A534, |
| | A535, A538, A540, A541, A542, A543, A547, A548, A549, A550, A551, A552, A553, A556, A557, |
| | A559, A563, A564, A566, A567, A568, A569, A570, A576, A579, A580, A581, A582, A583, |
| | A585, A586, A587, A588, A589, A591, A593, A595, A597, A598, A6, A601, A602, A603, A608, |
| | A610, A611, A614, A62, A64, A65, A66, A67, A68, A7, A76, A78, A79, A8, A80, A81, A82, A83, |
| | A84, A85, A87, A88, A89, A9, A90, A91, A93, A94, A99 |
| ++++ | A105, A107, A108, A109, A110, A112, A113, A115, A116, A118, A119, A12, A141, A142, A143, |
| | A150, A152, A175, A177, A178, A180, A181, A182, A183, A185, A199, A203, A206, A207, A213, |
| | A214, A220, A221, A222, A225, A225, A234, A235, A236, A237, A238, A239, A244, A245, |
| | A246, A253, A253, A256, A258, A261, A271, A272, A281, A282, A29, A3, A300, A305, A307, |
| | A317, A33, A34, A359, A36, A36, A366, A388, A389, A398, A4, A42, A425, A428, A429, A431, |
| | A442, A446, A448, A453, A460, A462, A464, A470, A479, A481, A482, A497, A499, A516, A517, |
| | A52, A53, A539, A54, A54, A544, A55, A56, A57, A572, A575, A58, A59, A592, A599, A60, A604, |
| | A609, A61, A612, A63, A69, A70, A75, A86, A92, A96, A97, A98, A613 |
| +++++ | A103, A103, A104, A104, A116, A117, A145, A153, A154, A172, A186, A187, A188, A189, A190, |
| | A191, A192, A193, A194, A195, A196, A197, A198, A200, A212, A327, A328, A340, A341, |
| | A430, A77 |

TABLE 13
KRAS G13D FRET data

| IC50* | Examples (Example A55 not tested) |
|---|---|
| + | None |
| ++ | A1, A10, A100, A111, A114, A121, A124, A125, A127, A128, A129, A130, A131, A133, A134, |
| | A135, A139, A140, A148, A151, A155, A159, A162, A163, A164, A165, A169, A2, A202, A204, |
| | A211, A216, A217, A223, A224, A227, A227, A228, A229, A23, A231, A242, A243, A247, A248, |
| | A249, A251, A252, A255, A27, A275, A277, A3, A323, A325, A342, A347, A348, A349, A365, |
| | A370, A371, A378, A379, A380, A381, A385, A386, A391, A392, A393, A395, A396, A397, A4, |
| | A405, A407, A409, A41, A415, A419, A424, A426, A427, A432, A45, A459, A501, A507, A529, |
| | A546, A558, A573, A577, A578, A584, A594, A605, A607, A615, A67, A87, A88, A95 |
| +++ | A101, A102, A11, A117, A119, A12, A120, A122, A123, A126, A13, A132, A136, A14, A146, A147, |
| | A149, A15, A156, A157, A158, A16, A160, A161, A166, A167, A168, A17, A170, A171, A174, |
| | A176, A179, A18, A184, A19, A20, A201, A205, A209, A21, A215, A218, A219, A226, A230, |
| | A232, A233, A236, A24, A240, A241, A248, A25, A250, A252, A254, A254, A257, A259, A26, |
| | A262, A264, A265, A266, A267, A268, A270, A273, A274, A278, A279, A28, A280, A284, A285, |
| | A286, A287, A288, A289, A290, A291, A293, A294, A295, A298, A302, A303, A304, A306, A309, |
| | A31, A310, A311, A312, A313, A314, A315, A316, A318, A319, A32, A321, A324, A329, A330, |
| | A332, A333, A334, A335, A336, A337, A338, A343, A345, A346, A35, A350, A351, A352, A353, |
| | A356, A357, A358, A361, A363, A364, A369, A372, A373, A374, A375, A376, A377, A38, A382, |
| | A383, A384, A387, A39, A394, A399, A4, A40, A400, A401, A402, A403, A404, A406, A408, |
| | A410, A411, A412, A413, A414, A416, A417, A418, A420, A421, A422, A423, A43, A433, A434, |
| | A435, A436, A437, A438, A441, A442, A443, A444, A445, A447, A450, A452, A454, A457, A458, |
| | A46, A463, A465, A466, A467, A468, A469, A47, A471, A473, A475, A476, A477, A478, A48, |
| | A480, A483, A484, A485, A486, A487, A488, A489, A491, A492, A493, A494, A498, A5, A500, |
| | A502, A503, A504, A505, A506, A509, A51, A510, A512, A513, A514, A515, A518, A520, A523, |
| | A525, A526, A528, A531, A532, A533, A534, A535, A536, A537, A538, A540, A542, A543, |
| | A545, A549, A550, A551, A552, A554, A555, A557, A559, A560, A561, A562, A563, A564, A565, |
| | A566, A567, A568, A569, A570, A571, A574, A576, A580, A581, A582, A583, A586, A587, A588, |
| | A589, A590, A591, A593, A595, A596, A597, A598, A6, A600, A601, A602, A603, A606, A608, |
| | A610, A611, A614, A616, A62, A64, A65, A66, A68, A7, A76, A78, A79, A8, A80, A81, A83, |
| | A84, A85, A87, A89, A9, A90, A93, A94 |
| ++++ | A105, A106, A107, A108, A109, A110, A112, A113, A115, A116, A118, A12, A137, A138, A141, |
| | A142, A143, A144, A152, A173, A175, A177, A178, A178, A179, A180, A181, A182, A183, A185, |
| | A199, A203, A206, A207, A208, A210, A213, A214, A22, A222, A225, A225, A236, A238, A239, |
| | A246, A253, A256, A258, A260, A261, A272, A276, A281, A282, A283, A29, A292, A296, |
| | A297, A299, A30, A300, A301, A305, A307, A308, A317, A320, A322, A326, A33, A331, A339, |
| | A34, A344, A354, A355, A359, A36, A36, A360, A362, A366, A367, A368, A37, A388, A389, |
| | A398, A42, A425, A431, A439, A44, A440, A446, A448, A449, A451, A455, A456, A460, A461, |
| | A462, A464, A470, A472, A474, A479, A481, A482, A49, A490, A495, A496, A497, A499, A50, |
| | A508, A511, A516, A517, A519, A52, A521, A522, A524, A527, A53, A530, A539, A54, A541, |
| | A544, A547, A548, A553, A556, A56, A57, A572, A575, A579, A58, A585, A59, A592, A599, A604, |
| | A609, A61, A612, A63, A69, A70, A75, A77, A82, A86, A91, A92, A96, A97, A98, A99, A613 |
| +++++ | A103, A103, A104, A104, A116, A117, A145, A150, A153, A154, A172, A186, A187, A188, A189, |
| | A190, A191, A192, A193, A194, A195, A196, A197, A198, A200, A212, A220, A221, A234, A235, |
| | A237, A244, A245, A253, A271, A3, A327, A328, A340, A341, A428, A429, A430, A453, A60 |

TABLE 14
KRAS Q61H FRET data

| IC50* | Examples |
|---|---|
| + | A159, A275, A415, A501, A546, A577, A594, A605 |
| ++ | A102, A124, A136, A174, A204, A205, A226, A230, A236, A24, A25, A250, A266, A268, A273, |
| | A274, A277, A278, A279, A280, A285, A287, A289, A290, A291, A3, A302, A304, A306, A309, |
| | A310, A312, A316, A324, A334, A335, A346, A349, A356, A358, A37, A372, A373, A374, A376, |
| | A38, A382, A383, A387, A396, A399, A400, A401, A405, A406, A409, A422, A424, A426, A434, |
| | A438, A443, A444, A450, A454, A457, A459, A465, A466, A467, A468, A471, A474, A475, A477, |
| | A478, A480, A483, A484, A485, A487, A488, A491, A492, A493, A494, A498, A500, A502, |
| | A503, A505, A507, A509, A510, A514, A515, A520, A523, A526, A528, A529, A530, A531, A533, |
| | A534, A536, A537, A538, A540, A543, A545, A549, A550, A551, A552, A554, A555, A557, |
| | A558, A559, A560, A561, A562, A563, A564, A565, A566, A567, A569, A571, A573, A574, A578, |
| | A581, A582, A583, A584, A589, A590, A591, A593, A595, A596, A600, A601, A606, A607, A608, |
| | A610, A614, A615, A616, A89 |
| +++ | A138, A144, A152, A163, A170, A176, A177, A178, A178, A179, A179, A180, A181, A207, A208, |
| | A210, A225, A225, A253, A264, A265, A267, A270, A271, A272, A276, A281, A282, A283, A284, |
| | A286, A293, A297, A299, A305, A315, A320, A321, A331, A337, A344, A354, A357, A359, |
| | A360, A361, A362, A366, A367, A368, A369, A375, A388, A389, A423, A433, A435, A436, A437, |
| | A439, A440, A441, A442, A445, A447, A449, A451, A452, A453, A455, A456, A458, A460, |
| | A461, A462, A463, A464, A469, A470, A472, A473, A476, A479, A481, A482, A486, A489, A490, |
| | A495, A496, A497, A499, A504, A506, A508, A511, A512, A513, A518, A519, A521, A522, A524, |
| | A525, A527, A532, A535, A54, A541, A542, A544, A547, A548, A553, A556, A568, A57, A570, |
| | A572, A576, A579, A580, A585, A586, A587, A588, A592, A597, A598, A599, A602, A603, |
| | A604, A609, A61, A611, A612, A99 |
| ++++ | A104, A145, A150, A220, A234, A237, A446, A448, A516, A517, A539, A575, A613 |
| +++++ | A154, A186, A189, A191, A328, A340 |

TABLE 15
NRAS G12C FRET data

| IC50* | Examples |
|---|---|
| + | A323, A325, A501, A577, A578, A594 |
| ++ | A1, A10, A100, A11, A114, A120, A121, A125, A127, A128, A129, A131, A135, A136, A139, A140, |
| | A146, A147, A148, A151, A156, A157, A159, A160, A162, A164, A165, A166, A168, A2, A201, |
| | A202, A204, A205, A211, A217, A218, A219, A223, A224, A228, A229, A230, A231, A233, |
| | A240, A242, A243, A247, A248, A248, A249, A250, A252, A252, A264, A265, A266, A267, A268, |
| | A273, A274, A275, A277, A278, A279, A280, A285, A288, A289, A290, A291, A298, A3, A302, |
| | A303, A304, A306, A309, A310, A312, A313, A316, A319, A321, A330, A333, A334, A335, A336, |
| | A338, A342, A343, A346, A347, A348, A349, A350, A351, A353, A356, A357, A358, A363, |
| | A364, A365, A370, A371, A372, A373, A374, A376, A377, A378, A379, A380, A381, A382, A383, |
| | A384, A385, A386, A387, A391, A392, A393, A395, A396, A397, A399, A4, A400, A401, A402, |
| | A405, A406, A407, A408, A409, A411, A413, A414, A415, A418, A419, A422, A424, A426, |
| | A427, A432, A436, A438, A443, A444, A450, A452, A454, A457, A458, A459, A465, A466, A467, |
| | A471, A475, A476, A477, A478, A483, A484, A487, A488, A489, A491, A493, A494, A498, A5, |
| | A500, A503, A505, A507, A509, A510, A514, A515, A523, A526, A528, A529, A531, A533, A534, |
| | A536, A537, A538, A540, A543, A545, A546, A549, A550, A551, A552, A554, A555, A557, |
| | A558, A559, A560, A561, A562, A565, A567, A569, A571, A573, A574, A576, A581, A582, A583, |
| | A584, A590, A591, A593, A595, A596, A598, A6, A600, A601, A605, A606, A607, A608, A614, |
| | A615, A616, A8, A87, A95 |
| +++ | A101, A102, A106, A111, A117, A122, A123, A124, A126, A13, A130, A132, A133, A134, A137, |
| | A138, A14, A149, A15, A155, A158, A16, A161, A163, A167, A169, A17, A170, A171, A173, A174, |
| | A176, A18, A184, A19, A20, A209, A21, A210, A215, A216, A226, A227, A227, A23, A232, A236, |
| | A24, A241, A25, A251, A254, A254, A255, A257, A259, A26, A260, A262, A27, A270, A276, |
| | A28, A282, A283, A284, A286, A287, A29, A292, A293, A294, A295, A296, A297, A299, A30, |
| | A301, A305, A308, A31, A311, A314, A315, A318, A32, A320, A322, A324, A326, A329, A331, |
| | A332, A337, A339, A344, A345, A35, A352, A354, A355, A359, A36, A36, A360, A361, A362, A366, |
| | A367, A368, A369, A37, A375, A38, A389, A39, A394, A40, A403, A404, A41, A410, A412, |
| | A416, A417, A420, A421, A423, A43, A431, A433, A434, A435, A437, A439, A44, A440, A441, |
| | A445, A447, A449, A451, A453, A455, A456, A46, A460, A461, A462, A463, A468, A469, A47, |
| | A472, A473, A474, A479, A480, A481, A482, A485, A486, A490, A492, A495, A496, A497, A499, |
| | A502, A504, A506, A508, A511, A512, A513, A518, A519, A520, A521, A522, A524, A525, A527, |
| | A530, A532, A535, A539, A541, A542, A544, A547, A548, A553, A556, A563, A564, A566, |
| | A568, A570, A579, A580, A585, A586, A587, A588, A589, A592, A597, A602, A603, A609, A610, |
| | A611, A612, A62, A64, A65, A66, A67, A68, A76, A78, A79, A80, A81, A83, A85, A87, A88, A89, |
| | A9, A90, A91, A93, A94, A96, A97, A98, A99 |
| ++++ | A105, A107, A108, A109, A110, A112, A113, A115, A116, A117, A118, A119, A12, A141, A142, |
| | A143, A144, A150, A152, A175, A177, A178, A178, A179, A179, A180, A181, A182, A183, A185, |
| | A199, A203, A206, A207, A208, A212, A213, A214, A220, A221, A225, A225, A235, A236, |
| | A237, A238, A239, A244, A245, A246, A253, A253, A256, A258, A261, A271, A272, A281, A3, |
| | A300, A307, A317, A388, A398, A4, A42, A425, A428, A429, A442, A446, A448, A45, A464, A470, |
| | A49, A50, A51, A516, A517, A52, A53, A54, A54, A56, A57, A572, A575, A58, A59, A599, A60, |
| | A604, A61, A63, A75, A82, A84, A86, A92, A613 |
| +++++ | A103, A103, A104, A104, A116, A145, A153, A154, A172, A186, A187, A188, A189, A190, A191, |
| | A192, A193, A194, A195, A196, A197, A198, A200, A222, A234, A327, A328, A340, A341, A430, |
| | A77 |

TABLE 16
NRAS WT FRET data

| IC50* | Examples |
|---|---|
| + | A501, A577, A594 |
| ++ | A124, A136, A159, A204, A230, A25, A250, A273, A274, A275, A277, A278, A279, A287, A290, |
| | A291, A3, A304, A306, A309, A310, A312, A335, A346, A349, A356, A372, A373, A374, A387, |
| | A396, A399, A400, A405, A406, A409, A415, A422, A424, A426, A434, A438, A443, A450, A454, |
| | A457, A459, A465, A466, A471, A475, A477, A478, A483, A484, A487, A491, A493, A494, |
| | A498, A500, A503, A505, A507, A526, A528, A529, A531, A533, A536, A537, A545, A546, A554, |
| | A555, A558, A560, A561, A562, A565, A569, A571, A573, A574, A578, A584, A590, A591, A596, |
| | A600, A605, A606, A607, A615 |
| +++ | A102, A138, A144, A163, A170, A174, A176, A178, A179, A179, A205, A208, A210, A225, A226, |
| | A236, A24, A253, A264, A265, A266, A267, A268, A270, A276, A280, A283, A284, A285, A286, |
| | A289, A293, A297, A299, A302, A305, A315, A316, A320, A321, A324, A331, A334, A337, |
| | A344, A354, A357, A358, A359, A360, A361, A362, A366, A367, A368, A369, A37, A375, A376, |
| | A38, A382, A383, A401, A423, A433, A435, A436, A437, A439, A440, A441, A444, A445, A447, |
| | A449, A451, A452, A455, A456, A458, A463, A467, A468, A469, A472, A473, A474, A476, A479, |
| | A480, A481, A485, A486, A488, A489, A490, A492, A495, A496, A497, A502, A504, A506, |
| | A508, A509, A510, A511, A512, A513, A514, A515, A518, A519, A520, A521, A522, A523, A524, |
| | A525, A527, A530, A532, A534, A535, A538, A540, A541, A542, A543, A547, A548, A549, |
| | A550, A551, A552, A553, A556, A557, A559, A563, A564, A566, A567, A568, A570, A576, A579, |
| | A580, A581, A582, A583, A585, A586, A587, A588, A589, A593, A595, A597, A598, A601, A602, |
| | A603, A608, A610, A611, A612, A614, A616, A89, A99 |
| ++++ | A104, A150, A152, A177, A178, A180, A181, A207, A220, A225, A234, A237, A271, A272, A281, |
| | A282, A388, A389, A442, A446, A448, A453, A460, A461, A462, A464, A470, A482, A499, A516, |
| | A517, A539, A54, A544, A57, A572, A575, A592, A599, A604, A609, A61, A613 |
| +++++ | A145, A154, A186, A189, A191, A328, A340 |

TABLE 17
NRAS Q61K FRET data

| IC50* | Examples |
|---|---|
| + | A275 |
| ++ | A136, A159, A170, A205, A266, A268, A277, A278, A279, A280, A285, A289, A290, A291, A302, |
| | A304, A306, A309, A310, A312, A316, A334, A335, A337, A344, A349, A356, A358, A372, |
| | A373, A374, A376, A382, A383, A387, A396, A399, A400, A401, A405, A409, A415, A422, A443, |
| | A444, A445, A450, A451, A452, A454, A457, A458, A459, A465, A466, A467, A471, A475, |
| | A477, A478, A483, A484, A485, A486, A487, A488, A489, A491, A493, A494, A500, A501, A503, |
| | A505, A510, A511, A512, A514, A515, A520, A522, A523, A526, A528, A529, A530, A531, |
| | A532, A533, A534, A536, A537, A538, A540, A543, A546, A549, A550, A551, A552, A555, A557, |
| | A560, A561, A565, A566, A567, A569, A571, A573, A574, A576, A577, A578, A580, A581, |
| | A582, A584, A590, A591, A594, A595, A596, A598, A600, A601, A606, A607, A608, A614 |
| +++ | A102, A124, A138, A144, A152, A174, A176, A204, A208, A210, A226, A230, A236, A24, A25, |
| | A250, A264, A265, A267, A270, A271, A273, A274, A276, A281, A283, A284, A286, A287, A293, |
| | A297, A299, A3, A305, A315, A320, A321, A324, A331, A346, A354, A357, A359, A360, |
| | A361, A362, A366, A367, A368, A369, A375, A38, A389, A406, A424, A426, A433, A434, A436, |
| | A437, A438, A439, A440, A441, A447, A449, A453, A455, A456, A460, A461, A462, A468, |
| | A469, A472, A474, A476, A479, A480, A481, A482, A490, A492, A495, A496, A497, A498, A499, |
| | A502, A506, A507, A509, A513, A521, A524, A525, A527, A535, A541, A542, A544, A545, |
| | A547, A548, A553, A554, A556, A558, A559, A562, A563, A564, A568, A570, A579, A583, A585, |
| | A586, A587, A588, A589, A592, A593, A597, A602, A603, A604, A605, A609, A610, A611, |
| | A612, A613, A615, A616, A89 |
| ++++ | A104, A150, A163, A177, A178, A178, A179, A179, A180, A181, A207, A220, A225, A225, A234, |
| | A237, A253, A272, A282, A37, A388, A423, A435, A442, A446, A448, A463, A464, A470, |
| | A473, A504, A508, A516, A517, A518, A519, A539, A54, A57, A572, A575, A599, A61, A99 |
| +++++ | A145, A154, A186, A189, A191, A328, A340 |

TABLE 18
NRAS Q61R FRET data

| IC50* | Examples |
|---|---|
| + | A577, A594 |
| ++ | A136, A159, A275, A277, A278, A279, A291, A304, A306, A309, A310, A312, A335, A349, A356, |
| | A372, A373, A374, A396, A399, A400, A415, A422, A450, A454, A459, A465, A466, A475, |
| | A477, A483, A487, A494, A500, A501, A503, A505, A520, A526, A528, A529, A536, A546, A555, |
| | A560, A561, A562, A565, A578, A583, A584, A593, A601, A606, A607 |
| +++ | A102, A138, A170, A174, A204, A205, A210, A226, A230, A24, A25, A250, A264, A265, A266, |
| | A267, A268, A270, A273, A274, A276, A280, A283, A285, A286, A287, A289, A290, A293, A297, |
| | A299, A3, A302, A305, A316, A321, A334, A337, A344, A346, A354, A357, A358, A361, |
| | A369, A375, A376, A38, A382, A383, A387, A401, A405, A406, A409, A423, A424, A426, A433, |
| | A434, A436, A437, A438, A439, A440, A441, A443, A444, A445, A449, A451, A452, A453, |
| | A455, A456, A457, A458, A461, A467, A468, A471, A472, A474, A476, A478, A480, A484, A485, |
| | A486, A488, A489, A490, A491, A492, A493, A495, A498, A499, A502, A506, A507, A509, |
| | A510, A511, A512, A513, A514, A515, A521, A522, A523, A525, A527, A530, A531, A532, A533, |
| | A534, A535, A537, A538, A540, A541, A542, A543, A545, A547, A548, A549, A550, A551, |
| | A552, A553, A554, A556, A557, A558, A559, A563, A564, A566, A567, A568, A569, A570, A571, |
| | A573, A574, A576, A579, A580, A581, A582, A585, A586, A587, A589, A590, A591, A595, |
| | A596, A597, A598, A600, A602, A603, A605, A608, A609, A610, A611, A614, A615, A616, A89 |
| ++++ | A124, A144, A152, A163, A176, A177, A178, A179, A179, A180, A207, A208, A220, A225, A225, |
| | A236, A237, A253, A271, A272, A281, A282, A284, A315, A320, A324, A331, A359, A360, |
| | A362, A366, A367, A368, A37, A388, A389, A435, A442, A447, A448, A460, A462, A463, A464, |
| | A469, A470, A473, A479, A481, A482, A496, A497, A504, A508, A516, A517, A518, A519, |
| | A524, A539, A54, A544, A57, A572, A575, A588, A592, A599, A604, A61, A612, A99, A613 |
| +++++ | A104, A145, A150, A154, A178, A181, A186, A189, A191, A234, A328, A340, A446 |

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC₅₀).

The assay took place over 7 days. On day 1, cells in 2D culture were harvested during logarithmic growth and suspended in culture medium at 1×105 cells/ml. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% CO₂. On day 2, one plate (for to reading) was removed and 10 μl growth medium plus 100 μl CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 37C and 5% CO₂. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC₅₀ for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite having a KRAS G12C mutation, is insensitive to the KRAS G12C(OFF) inhibitor MRTX-849 (Hallin et al., Cancer Discovery 10:54-71 (2020)), and the cell line SW1573, which is insensitive to the KRAS G12C(OFF) inhibitor AMG510 (Canon et al., Nature 575:217-223 (2019)).

TABLE 19
IC50 values for various cancer cell lines
with Compound B, Compound C, and Compound D

| Cell | | | Cmpd B | Cmpd C | Cmpd D |
|---|---|---|---|---|---|
| Line | Histotype | Mutant | IC50* | IC50* | IC50* |
| A-375 | Skin | BRAF V600E | low | low | low |
| | | | sensitivity | sensitivity | sensitivity |
| KYSE- | HN/Esophagus | KRAS G12C | moderately | | very |
| 410 | | | sensitive | | sensitive |
| MIA | Pancreas | KRAS G12C | moderately | very | very |
| PaCa-2 | | | sensitive | sensitive | sensitive |
| NCI- | Lung | KRAS G12C | moderately | very | very |
| H358 | | | sensitive | sensitive | sensitive |
| SW1573 | Lung | KRAS G12C | low | low | low |
| | | | sensitivity | sensitivity | sensitivity |
| SW837 | Intestine/Large/ | KRAS G12C | moderately | | moderately |
| | Colorectum | | sensitive | | sensitive |
| LS513 | Intestine/Large/ | KRAS G12D | moderately | | moderately |
| | Colorectum | | sensitive | | sensitive |
| HuCCT1 | Liver/Bile duct | KRAS G12D | moderately | | very |
| | | | sensitive | | sensitive |
| HCC1588 | Lung | KRAS G12D | low | low | moderately |
| | | | sensitivity | sensitivity | sensitive |
| HPAC | Pancreas | KRAS G12D | moderately | | very |
| | | | sensitive | | sensitive |
| AsPC-1 | Pancreas | KRAS G12D | moderately | moderately | moderately |
| | | | sensitive | sensitive | sensitive |
| AGS | Stomach | KRAS G12D | moderately | very | moderately |
| | | | sensitive | sensitive | sensitive |
| HEC-1-A | Uterus | KRAS G12D | moderately | | moderately |
| | | | sensitive | | sensitive |
| SW403 | Intestine/Large/ | KRAS G12V | moderately | | very |
| | Colorectum | | sensitive | | sensitive |
| NOZ | Liver/Bile duct | KRAS G12V | moderately | | moderately |
| | | | sensitive | | sensitive |
| NCI- | Lung | KRAS G12V | moderately | moderately | moderately |
| H441 | | | sensitive | sensitive | sensitive |
| NCI- | Lung | KRAS G12V | moderately | very | very |
| H727 | | | sensitive | sensitive | sensitive |
| OVCAR-5 | Ovary | KRAS G12V | moderately | | very |
| | | | sensitive | | sensitive |
| Capan-2 | Pancreas | KRAS G12V | moderately | | very |
| | | | sensitive | | sensitive |
| SW48 | Intestine/Large/ | not MAPK | low | low | low |
| | Colorectum | (PIK3CA | sensitivity | sensitivity | sensitivity |
| | | G914R, | | | |
| | | EGFR | | | |
| | | G719S) | | | |
| NCI- | Lung | other KRAS | moderately | | moderately |
| H2009 | | (G12A) | sensitive | | sensitive |
| CAL-62 | HN/Thyroid | other KRAS | moderately | | |
| | | (G12R) | sensitive | | |
| A549 | Lung | other KRAS | moderately | moderately | moderately |
| | | (G12S) | sensitive | sensitive | sensitive |
| TOV-21G | Ovary | other KRAS | low | | moderately |
| | | (G13C) | sensitivity | | sensitive |
| DV-90 | Lung | other KRAS | low | | moderately |
| | | (G13D) | sensitivity | | sensitive |
| HCT116 | Intestine/Large/ | other KRAS | moderately | | very |
| | Colorectum | (G13D) | sensitive | | sensitive |
| NCI- | Intestine/Large/ | other KRAS | moderately | | very |
| H747 | Colorectum | (G13D) | sensitive | | sensitive |
| NCI- | Lung | other KRAS | moderately | moderately | moderately |
| H460 | | (Q61H) | sensitive | sensitive | sensitive |
| Calu-6 | Lung | other KRAS | moderately | very | moderately |
| | | (Q61K) | sensitive | sensitive | sensitive |
| SNU-668 | Stomach | other KRAS | moderately | | very |
| | | (Q61K) | sensitive | | sensitive |
| OZ | Liver/Bile duct | other KRAS | moderately | | moderately |
| | | (Q61L) | sensitive | | sensitive |
| SW948 | Intestine/Large/ | other KRAS | low | moderately | moderately |
| | Colorectum | (Q61L) | sensitivity | sensitive | sensitive |
| BxPC-3 | Pancreas | other MAPK | low | low | low |
| | | (BRAF | sensitivity | sensitivity | sensitivity |
| | | V487_{—} | | | |
| | | P492delinsA) | | | |
| NCI- | Lung | other MAPK | moderately | moderately | very |
| H1975 | | (EGFR | sensitive | sensitive | sensitive |
| | | T790M, | | | |
| | | L858R) | | | |
| NCI- | Lung | other MAPK | moderately | | moderately |
| H3122 | | (EML4- | sensitive | | sensitive |
| | | ALK(E13, | | | |
| | | A20)) | | | |
| YCC-1 | Stomach | other MAPK | | | moderately |
| | | (KRAS Amp) | | | sensitive |
| MeWo | Skin | other MAPK | low | moderately | moderately |
| | | (NF1 mut) | sensitivity | sensitive | sensitive |
| NCI- | Lung | other MAPK | moderately | moderately | moderately |
| H1838 | | (NF1 mut) | sensitive | sensitive | sensitive |
| RL95-2 | Uterus | other RAS | | | very |
| | | (HRAS Q61H) | | | sensitive |
| NCI- | Lung | other RAS | | moderately | moderately |
| H1915 | | (HRAS Q61L) | | sensitive | sensitive |
| L-363 | Blood/Leukemia | other RAS | | | low |
| | | (NRAS Q61H) | | | sensitivity |
| CHP-212 | Brain&Nerves | other RAS | | | moderately |
| | | (NRAS Q61K) | | | sensitive |
| HT-1080 | Soft tissue | other RAS | | | moderately |
| | | (NRAS Q61K) | | | sensitive |
| NCI- | Lung | other RAS | | | very |
| H2087 | | (NRAS Q61K) | | | sensitive |
| OCI-LY- | Blood/Lymphoma | other RAS | | | moderately |
| 19 | | (NRAS Q61K) | | | sensitive |
| SNU-387 | Liver/bile duct | other RAS | | | moderately |
| | | (NRAS Q61K) | | | sensitive |
| Hep G2 | Liver/bile duct | other RAS | | moderately | very |
| | | (NRAS Q61L) | | sensitive | sensitive |
| HL-60 | Blood/Leukemia | other RAS | | | very |
| | | (NRAS Q61L) | | | sensitive |
| MOLP8 | Blood/Myeloma | other RAS | | | moderately |
| | | (NRAS Q61L) | | | sensitive |
| SNU-719 | Stomach | other RAS | | | moderately |
| | | (NRAS Q61L) | | | sensitive |
| TF-1 | Blood/Leukemia | other RAS | | | moderately |
| | | (NRAS Q61P) | | | sensitive |
| ASH-3 | HN/Thyroid | other RAS | | | moderately |
| | | (NRAS Q61R) | | | sensitive |
| SK-MEL- | Skin | other RAS | | | moderately |
| 2 | | (NRAS Q61R) | | | sensitive |
| SW1271 | Lung | other RAS | | | moderately |
| | | (NRAS Q61R) | | | sensitive |

*Key:
low sensitivity: IC50 ≥ 1 uM
moderately sensitive: 1 uM > IC50 ≥ 0.1 uM
very sensitive: IC50 < 0.1 uM

In Vivo PD and Efficacy Data with Compound a, a Compound of the Present Invention

### FIG. 1A:

Methods: The human pancreatic adenocarcinoma Capan-2 KRASG12V/wt xenograft model was used for a single-day treatment PK/PD study (FIG. 1A). Compound A (Capan-2 pERK K-Ras G12D EC50: 0.0037 μM) was administered at 100 mg/kg as a single dose or bid (second dose administered 8 hours after first dose) orally administered (po). The treatment groups with sample collections at various time points were summarized in Table 20 below. Tumor samples were collected to assess RAS/ERK signaling pathway modulation by measuring the mRNA level of human DUSP6 in qPCR assay, while accompanying blood plasma samples were collected to measure circulating Compound A levels.

TABLE 20
Summary of treatment groups, doses, and time points
for single-dose PK/PD study using Capan-2 tumors.

| | | PK, n = 3/ | PD, n = 3/ |
|---|---|---|---|
| Compound/group | Dose/Regimen | time point | time point |
| Vehicle control | 10 ml/kg ip | 1 h, 24 h | 1 h, 24 h |
| Compound A | 100 mg/kg po | 1 h, 2 h, 8 h, | 1 h, 2 h, 8 h, |
| | | 12 h, 24 h | 10 h, 24 h |
| Compound A | 100 mg/kg po bid | 1 h, 2 h, 8 h, | 1 h, 2 h, 8 h, |
| | | 12 h, 24 h | 10 h, 24 h |

Results: In FIG. 1A, Compound A delivered at 100 mg/kg as a single dose inhibited DUSP6 mRNA levels in tumors >95% through 10 hours. A second dose of Compound A 8 hours following first administration maintained pathway modulation of 93% through 24 hours. These data indicate Compound A provides strong MAPK pathway modulation with continued target coverage.

### FIG. 1B:

Methods: Effects of Compound A on tumor cell growth in vivo were evaluated in the human pancreatic adenocarcinoma Capan-2 KRASG12V/wt xenograft model using female BALB/c nude mice (6-8 weeks old). Mice were implanted with Capan-2 tumor cells in 50% Matrigel (4×106 cells/mouse) subcutaneously in the flank. Once tumors reached an average size of ˜180 mm3, mice were randomized to treatment groups to start the administration of test articles or vehicle. Compound A was orally administered (po) twice daily at 100 mg/kg. A SHP2 inhibitor, RMC-4550 (commercially available), was administered orally every other day at 20 mg/kg. Body weight and tumor volume (using calipers) was measured twice weekly until study endpoints. Tumor regressions calculated as >10% decrease in starting tumor volume. All dosing arms were well tolerated.

Results: In FIG. 1B, single agent SHP2i RMC-4550 dosed every other day at 20 mg/kg po resulted in 39% TGI. Single-agent Compound A administered at 100 mg/kg po bid daily led to a TGI of 98%, with 4/10 (40%) individual animals achieving tumor regressions. Combination of Compound A and RMC-4550 resulted in total tumor regression of 35%, with individual tumor regressions observed in 7/9 (77.8%) individual animals at the end of treatment (Day 40 after treatment started) in Capan-2 CDX model with heterozygous KRASG12V. The anti-tumor activity of Compound A, and Combination arms was statistically significant compared with control group (*** p<0.001, ordinary One-way ANOVA with multiple comparisons via a post-hoc Tukey's test), while RMC-4550 was not significant at these doses.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX B

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### Summary

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

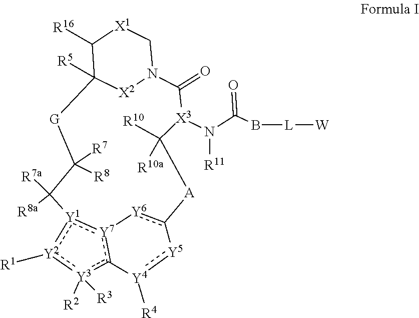

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

Also provided are pharmaceutical compositions comprising a compound of Formula I, or a pharmaceutically acceptable salt thereof, and a pharmaceutically acceptable excipient.

Further provided is a conjugate, or salt thereof, comprising the structure of Formula IV:

M-L-P Formula IV
wherein L is a linker;
P is a monovalent organic moiety; and
M has the structure of Formula V:

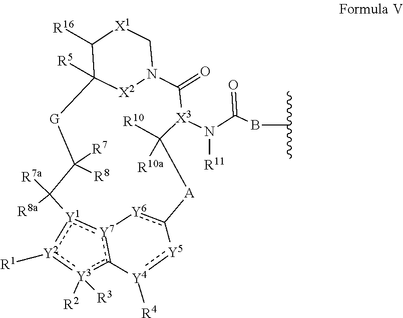

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is H or C₁-C₃ alkyl.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE DRAWINGS

FIG. 1A and FIG. 1B: These figures illustrate a matched pair analysis of potencies of certain compounds of the present invention (Formula BB) (points on the right) and corresponding compounds of Formula AA (points on the left) wherein a H is replaced with(S) Me in the context of two different cell-based assays. The y axes represent pERK EC50 (FIG. 1A) or CTG IC₅₀ (FIG. 1B) as measured in an H358 cell line.

FIG. 2A and FIG. 2B: A compound of the present invention, Compound A, drove deep regressions in vivo in a NSCLC(KRAS G12C) xenograft model. Some animals exhibited complete responses (CR)=3 consecutive tumor measurements≤30 mm3. FIG. 2A shows Compound A dosed at 100 mg/kg by daily oral gavage led to tumor regression in NCI-H358 KRAS^{G12C} xenograft model, which is a sensitive model to KRAS^{G12C} inhibition alone. The spaghetti titer plot (FIG. 2B) displaying individual tumor growth is shown next to the tumor volume plot (FIG. 2A).

FIG. 3A and FIG. 3B: A compound of the present invention, Compound B, drove tumor xenograft regressions in combination with a MEK inhibitor, cobimetinib, in a NSCLC(KRAS G12C) model. FIG. 3A shows the combination of intermittent intravenous administration of Compound B at 50 mg/kg plus daily oral administration of cobimetinib at 2.5 mg/kg drove tumor regression, whereas each single agent led to tumor growth inhibition. End of study responses were shown as waterfall plots (FIG. 3B), which indicate 6 out 10 mice had tumor regression in the combination group, whereas no tumor regressions recorded in each single agent group.

FIG. 4A and FIG. 4B: A compound of the present invention, Compound C, dosed weekly with daily SHP2 inhibitor, RMC-4550, drove xenograft regressions in a NSCLC(KRAS G12C) model. In FIG. 4A, the combinatorial activity of once weekly intravenous administration of Compound C at 60 mg/kg plus daily oral administration of SHP2 inhibitor at 30 mg/kg is shown. End of study responses in individual tumors were plotted as a waterfall plot (FIG. 4B).

FIG. 5: A compound of the present invention, Compound D, combined with a MEK inhibitor, trametinib, suppressed in vitro growth durably in a long-term cell growth NSCLC(KRAS G12C) model.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium;
halogen; —(CH₂)₀₋₄R^{∘}; —(CH₂)₀₋₄OR^{∘}; —O (CH₂)₀₋₄R^{∘};
—O—(CH₂)₀₋₄C(O)OR^{∘}; —(CH₂)₀₋₄CH (OR^{∘})₂; —(CH₂)₀₋₄SR^{∘}; —(CH₂)₀₋₄Ph, which may be substituted with R^{∘}; —(CH₂)₀₋₄O (CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or
cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S) R^{∘};)
(CH₂)₀₋₄N(R^{∘}C(O)NR^{∘}₂; —N(R^{∘}C(S)NR^{∘}₂; —(CH₂)₀₋₄N(R^{∘}C(O)OR^{∘}; —N(R^{∘}N(R^{∘}C(O)R^{∘}; —N(R^{∘}N(R^{∘}) C(O)NR^{∘}₂; —N(R^{∘}N(R^{∘}C(O)OR^{∘}; —(CH₂)₀₋₄C(O)R^{∘}; —C(S) R^{∘}; —(CH₂)₀₋₄C(O)OR^{∘}; —(CH₂)₀₋₄—C(O)—N(R^{∘})₂; —(CH₂)₀₋₄—C(O)—N(R^{∘}—S(O)₂—R^{∘}; —C(NCN) NR^{∘}₂; —(CH₂)₀₋₄C(O) SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S) SR^{∘}; —(CH₂)₀₋₄SC(O)R^{∘}; —(CH₂)₀₋₄C(O)NR^{∘}₂; —C(S) NR^{∘}₂; —C(S) SR^{∘}; —(CH₂)₀₋₄OC(O)NR^{∘}₂; —C(O)N (OR^{∘}) R^{∘}; —C(O) C(O)R^{∘}; —C(O) CH₂C(O)R^{∘}; —C(NOR^{∘}) R^{∘}; —(CH₂)_{0 -4}SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘}) R^{∘}; —C(NOR^{∘}) NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘}) R^{∘}, —SiR^{∘}₃; —(C₁₋₄ straight or branched) alkylene)O—N(R^{∘}₂; or —(C₁₋₄ straight or branched) alkylene) C(O)O—N(R^{∘}₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•},
(haloR^{•}), —(CH₂)₀₋₂OH, —(CH₂)₀₋₂OR*, —(CH₂)₀₋₂CH (OR^{•})₂; —O (haloR^{•}), —CN, —N₃, —(CH₂)₀₋₂C(O)R^{•}, —(CH₂)₀₋₂C(O)OH, (CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{•}, —(C₁₋₄ straight or branched alkylene) C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include:—O (CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include
halogen, —R^{•}-(haloR^{•}), —OH, —OR^{•},
—O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O) C(O)R^{†}, —C(O) CH₂C(O)R^{†}, —S(O)₂R^{†}, —S(O)₂NR^{†}₂, —C(S) NR^{†}₂, —C(N H) NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}-(haloR^{•}), —OH, —OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group-C(O) CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

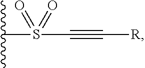

wherein R is any chemically feasible substituent described herein.

The term “amino,” as used herein, represents-N(R+)₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(Co alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

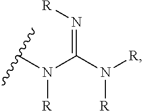

The term “guanidinyl,” refers to a group having the structure: wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more-OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC₅₀ of 2 μM or less in the Ras-RAF disruption assay protocol provided in the Examples below, and provided here:

The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras: RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

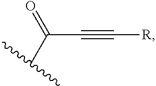

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that both covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., a sulfhydryl side chain of the cysteine at position 12 or 13 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art. One method of determining covalent adduct formation is to perform a “cross-linking” assay, such as under these conditions (Note—the following protocol describes a procedure for monitoring cross-linking of K-Ras G12C (GMP-PNP) to a compound of the invention. This protocol may also be executed substituting other Ras proteins or nucleotides).

The purpose of this biochemical assay is to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A and 2 μM test compound, a 5 μM stock of GMP-PNP-loaded K-Ras (1-169)^{G12C} is diluted 10-fold to yield a final concentration of 0.5 μM; with final sample volume being 100 μL.

The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras.

Accordingly, provided herein is a compound, or pharmaceutically acceptable salt thereof, having the structure of Formula I:

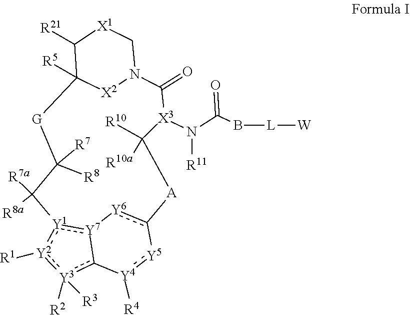

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, R²¹ is hydrogen.

In some embodiments, provided herein is a compound, or pharmaceutically acceptable salt thereof, having the structure of Formula Ia:

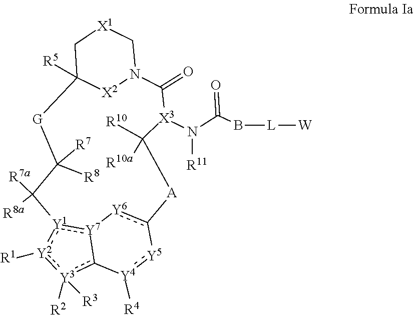

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or
S(O)₂N(R′)₂;
each R′ is, independently, H or optionally substituted C₁-C₄ alkyl; Y¹ is C, CH, or N; Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or

R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula Ib:

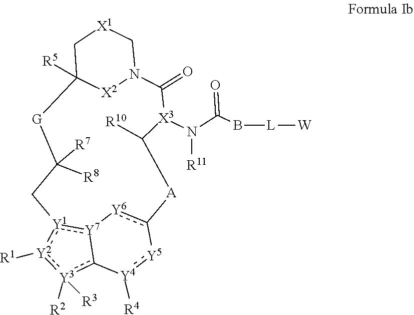

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, G is optionally substituted C₁-C₄ heteroalkylene.

In some embodiments, a compound having the structure of Formula Ic is provided, or a pharmaceutically acceptable salt thereof:

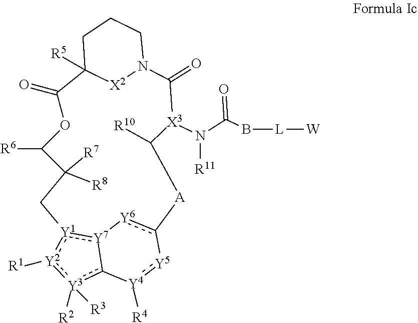

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, X² is NH. In some embodiments, X³ is CH. In some embodiments, R¹¹ is hydrogen. In some embodiments, R¹¹ is C₁-C₃ alkyl. In some embodiments, R¹¹ is methyl.

In some embodiments, a compound of the present invention has the structure of Formula Id, or a pharmaceutically acceptable salt thereof:

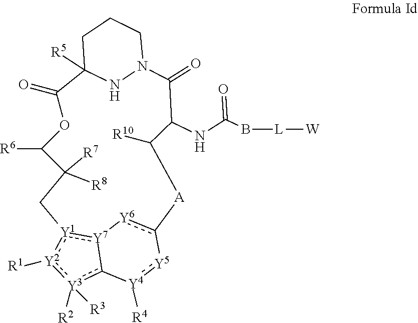

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of a compound of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH (Br)—. In some embodiments, X¹ is —CH (CH₃)—. In some embodiments, R⁵ is hydrogen. In some embodiments, R⁵ is C₁-C₄ alkyl optionally substituted with halogen. In some embodiments, R⁵ is methyl. In some embodiments, Y⁴ is C. In some embodiments, R⁴ is hydrogen. In some embodiments, Y⁵ is CH. In some embodiments, Y⁶ is CH. In some embodiments, Y¹ is C. In some embodiments, Y² is C. In some embodiments, Y³ is N. In some embodiments, R³ is absent. In some embodiments, Y⁷ is C.

In some embodiments, a compound of the present invention has the structure of Formula Ie, or a pharmaceutically acceptable salt thereof:

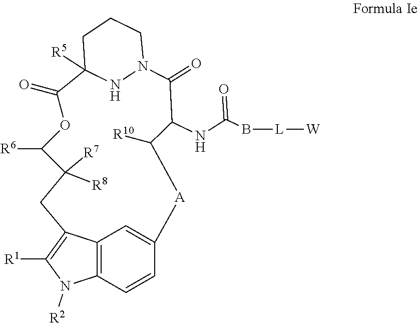

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.

In some embodiments of a compound of the present invention, R⁶ is hydrogen. In some embodiments, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R² is fluoroalkyl. In some embodiments, R² is ethyl. In some embodiments, R² is —CH₂CF₃. In some embodiments, R² is C₂-C₆ alkynyl. In some embodiments, R² is —CHC═CH. In some embodiments, R² is —CH₂C═CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃ alkyl.

In some embodiments, a compound of the present invention has the structure of Formula If, or a pharmaceutically acceptable salt thereof:

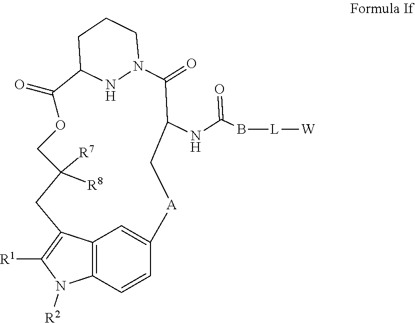

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a compound of the present invention, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of a compound of the present invention, R¹ is

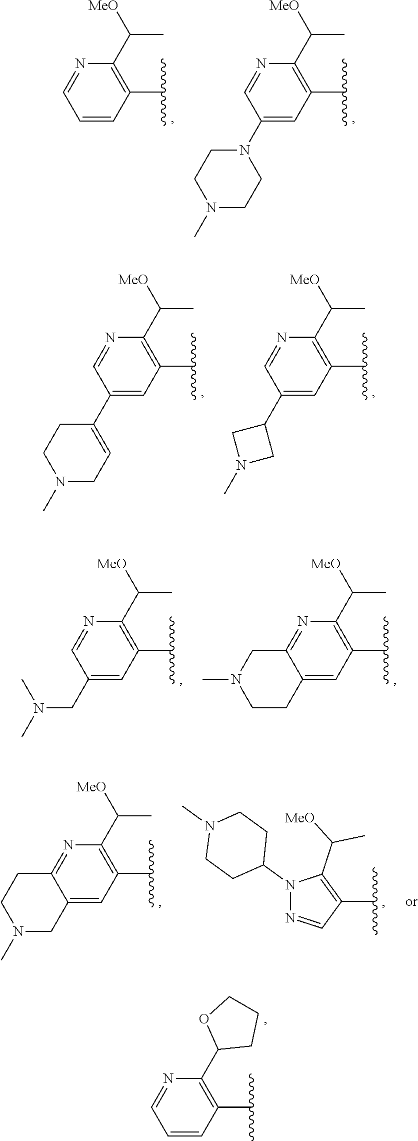

or a stereoisomer (e.g., atropisomer) thereof. In some embodiments of a compound of the present invention, R¹ is

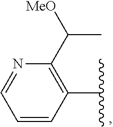 [SMILES: *c1cccnc1C(C)OC]

or a stereoisomer (e.g., atropisomer) thereof. In some embodiments of a compound of the present invention, R¹ is

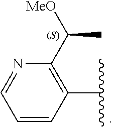

In some embodiments, a compound of the present invention has the structure of Formula Ig, or a pharmaceutically acceptable salt thereof:

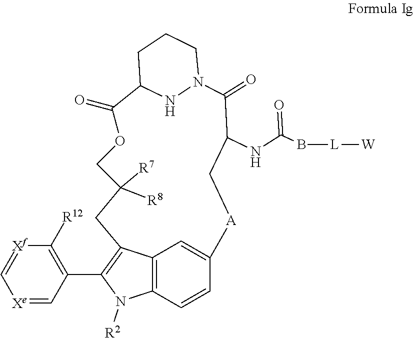

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} and X^{f} are, independently, N or CH; and
R¹² is optionally substituted C₁-C₆ alkyl, optionally substituted C₁-C₆ heteroalkyl, or optionally substituted 3 to 6-membered heterocycloalkylene.

In some embodiments of a compound of the present invention, X^{e} is N and X^{f} is CH. In some embodiments, X^{e} is CH and X^{f} is N.

In some embodiments of a compound of the present invention, R¹² is optionally substituted C₁-C₆ heteroalkyl. In some embodiments, R¹² is

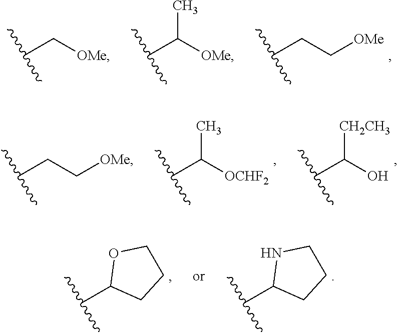

In some embodiments, R¹² is

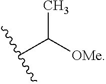 [SMILES: *C(C)OC]

In some embodiments, a compound of the present invention has the structure of Formula VI, or a pharmaceutically acceptable salt thereof:

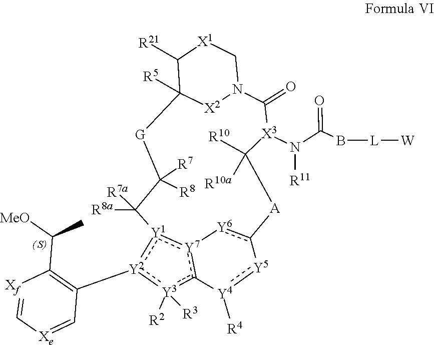

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, a compound of the present invention has the structure of Formula VIa, or a pharmaceutically acceptable salt thereof:

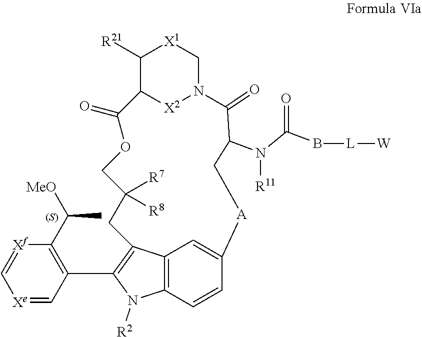

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of Formula VIb, or a pharmaceutically acceptable salt thereof:

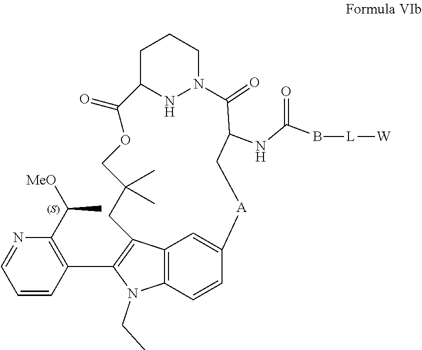

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone. In some embodiments of a compound of the present invention, A is optionally substituted 6-membered arylene.

In some embodiments, a compound of the present invention has the structure of Formula VIc (corresponding for Formula BB of FIG. 1A and FIG. 1B), or a pharmaceutically acceptable salt thereof:

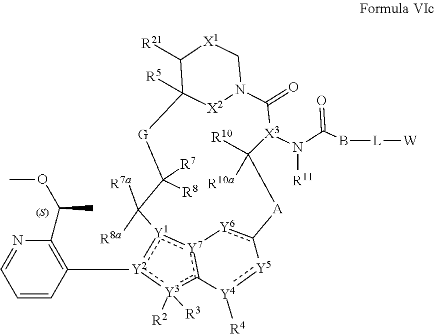

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments, A has the structure:

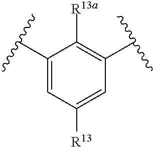

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments, A is optionally substituted 5 to 6-membered heteroarylene. In some embodiments, A is:

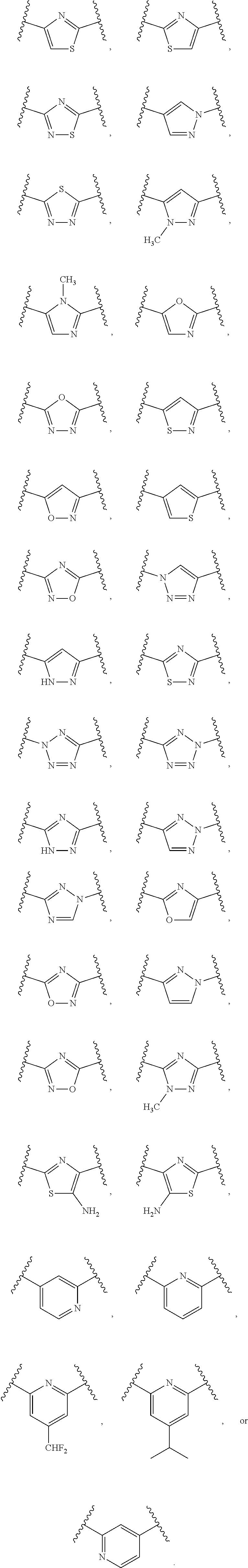

In some embodiments, A is optionally substituted C₁-C₄ heteroalkylene. In some embodiments, A is:

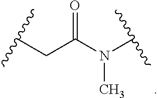 [SMILES: *CC(=O)N(*)C]

In some embodiments, A is optionally substituted 3 to 6-membered heterocycloalkylene. In some embodiments, A is:

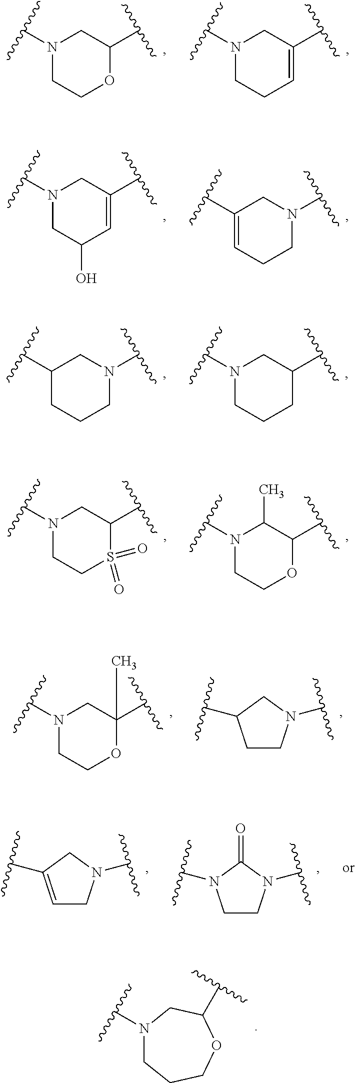

In some embodiments, A is

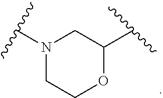 [SMILES: *C1CN(*)CCO1]

In some embodiments of a compound of the present invention, B is —CHR⁹—. In some embodiments, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

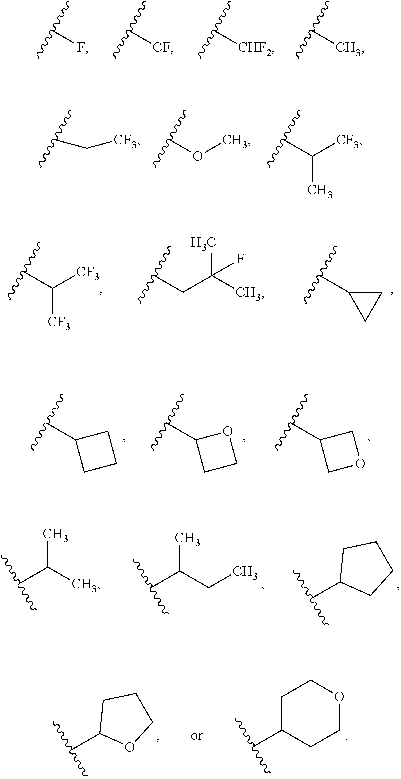

In some embodiments, R⁹ is:

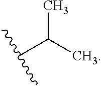 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a compound of the present invention, B is optionally substituted 6-membered arylene. In some embodiments, B is 6-membered arylene. In some embodiments, B is:

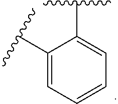 [SMILES: *c1ccccc1*]

In some embodiments of a compound of the present invention, R⁷ is methyl.

In some embodiments of a compound of the present invention, R⁸ is methyl.

In some embodiments, R²¹ is hydrogen.

In some embodiments of a compound of the present invention, the linker is the structure of Formula II:

A¹-(B¹)_{f}—(C¹)_{g}—(B²)ₕ-(D¹)-(B³)ᵢ —(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A². In some embodiments, the linker is acyclic. In some embodiments, linker has the structure of Formula IIa:

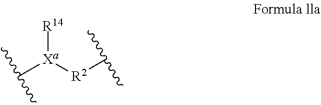

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

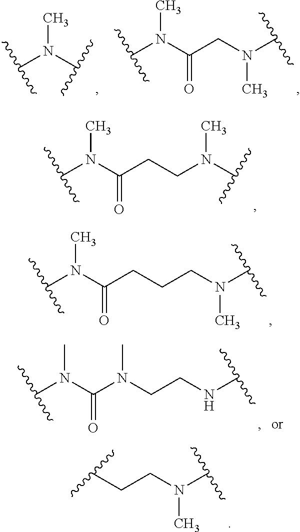

In some embodiments, the linker is or comprises a cyclic moiety. In some embodiments, the linker has the structure of Formula IIb:

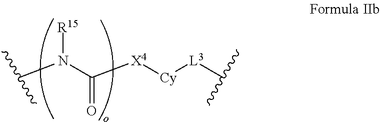

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments, the linker has the structure of Formula IIb-1:

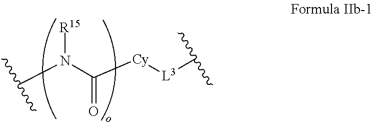

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments, the linker has the structure of Formula IIc:

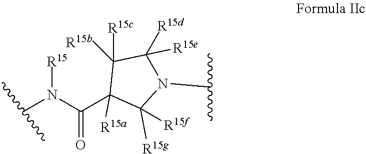

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a} R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments, the linker has the structure:

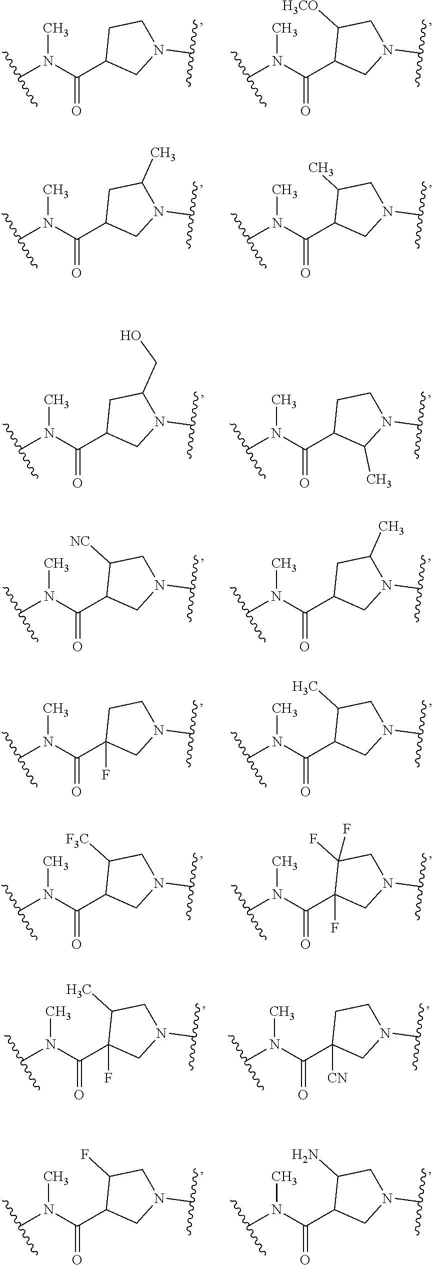

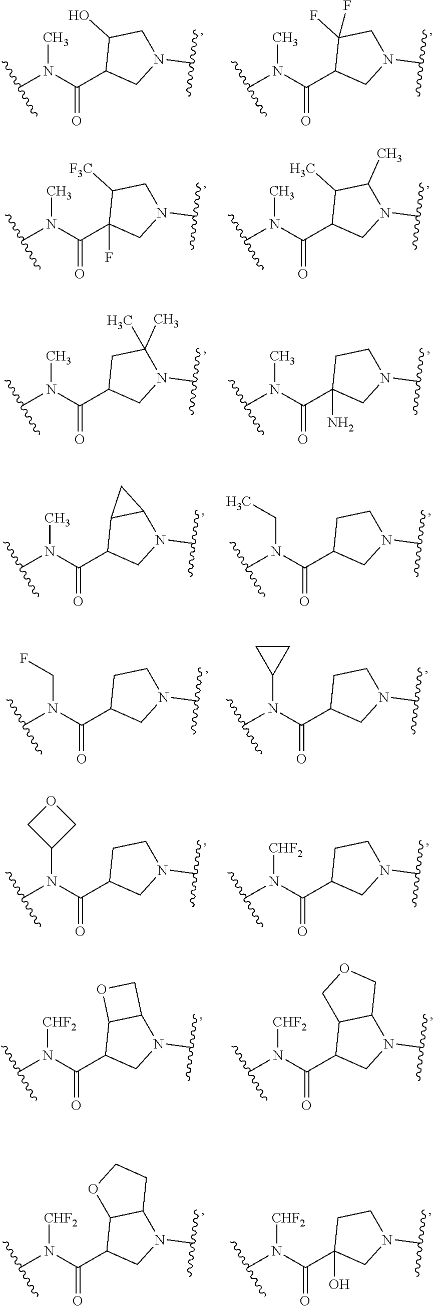

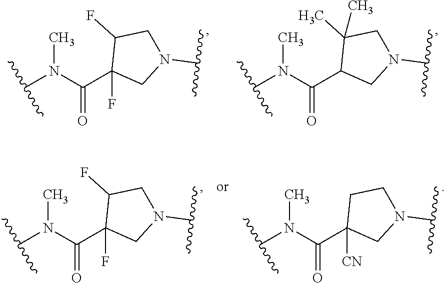 [SMILES: *N1CC(F)C(F)(C(=O)N(*)C)C1 ; *N1CC(C(=O)N(*)C)C(C)(C)C1 ; *N1CC(F)C(F)(C(=O)N(*)C)C1 ; *N1CCC(C#N)(C(=O)N(*)C)C1]

In some embodiments, the linker has the structure:

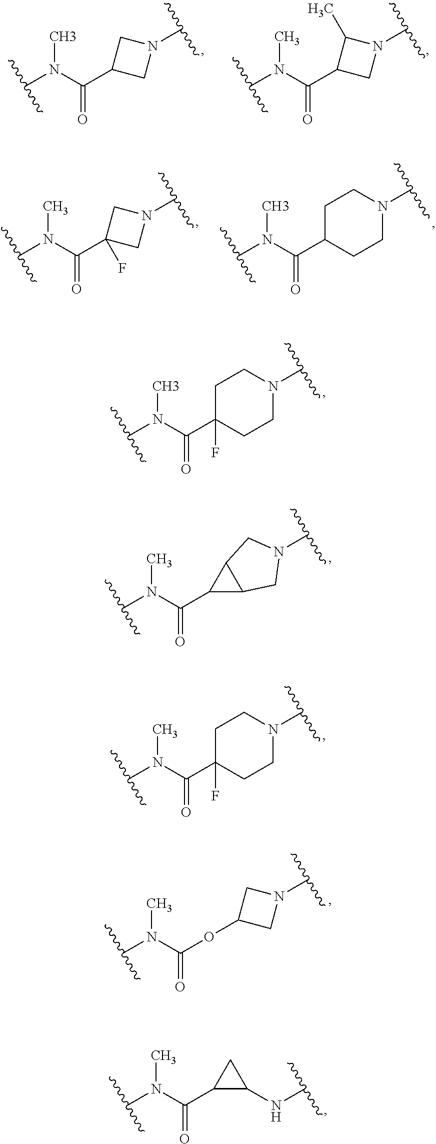

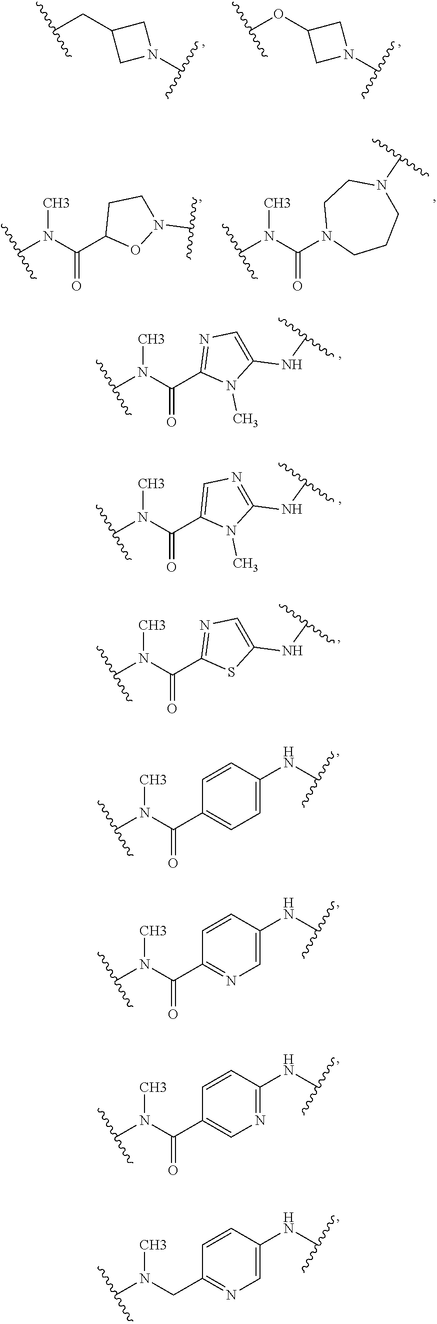

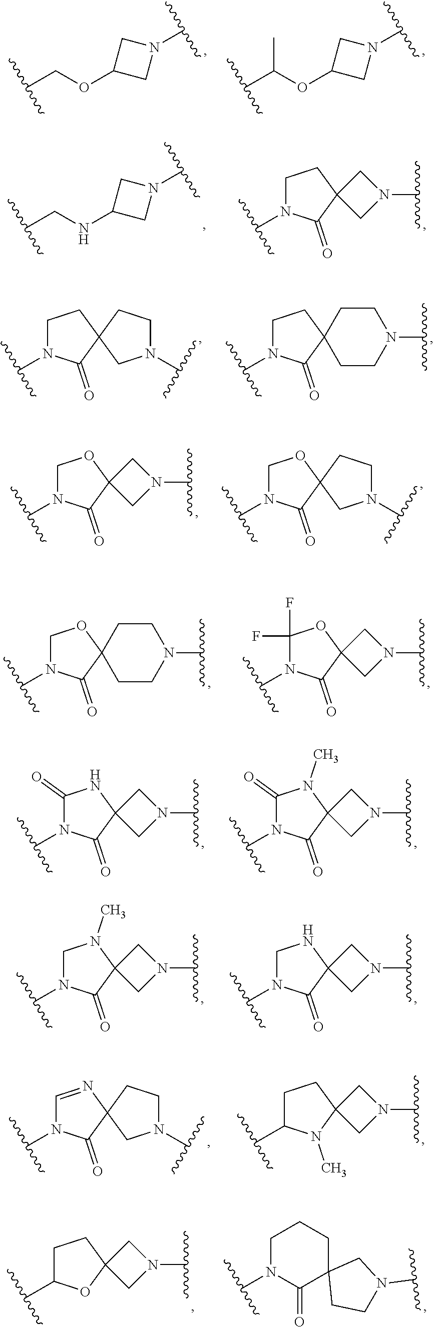

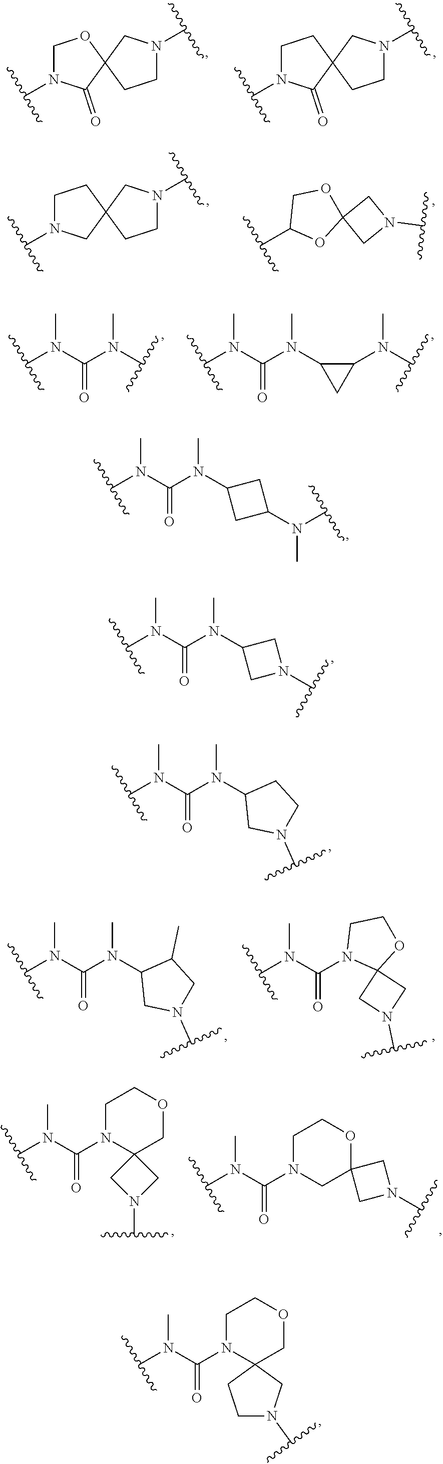

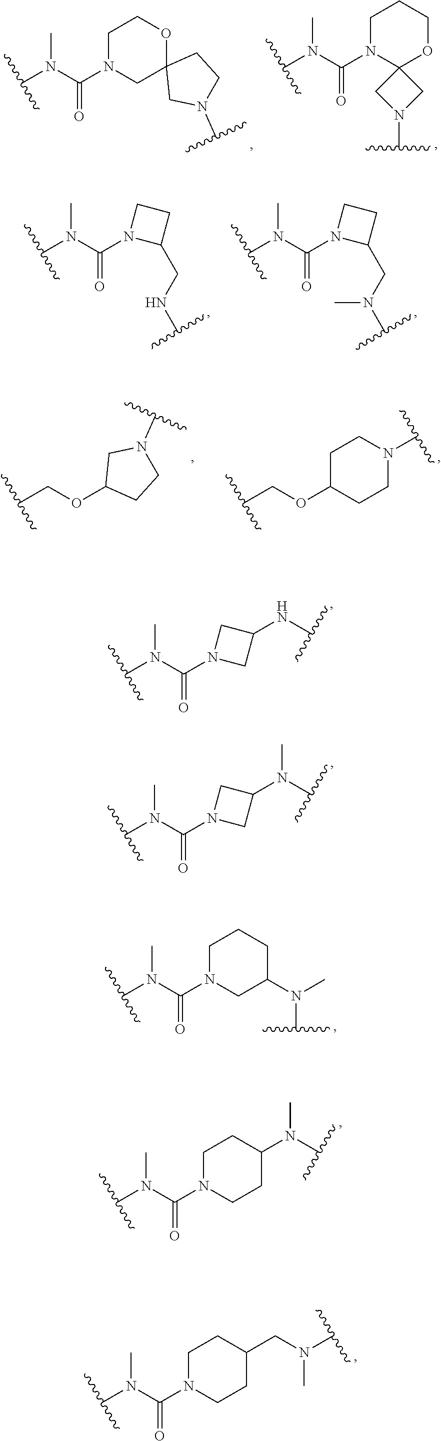

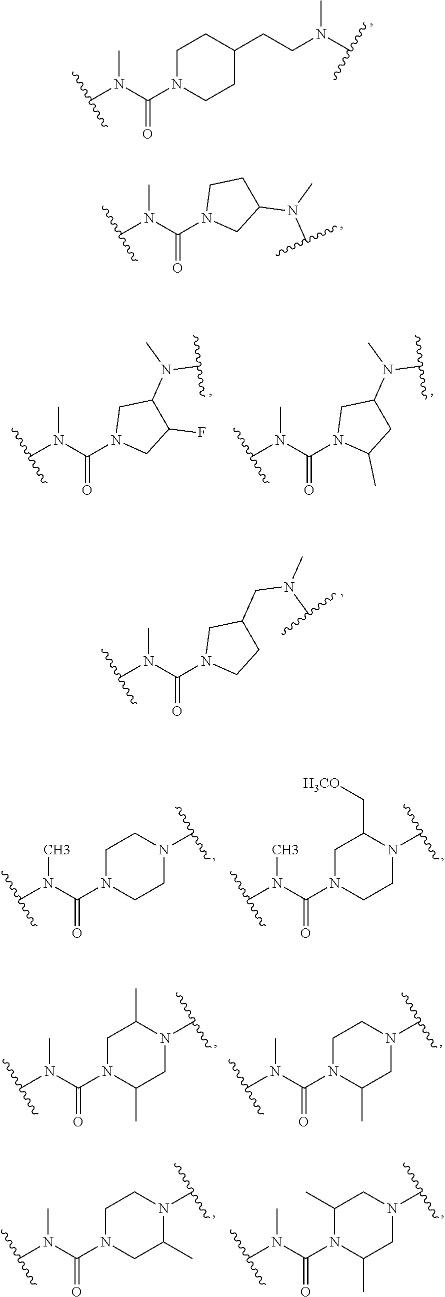

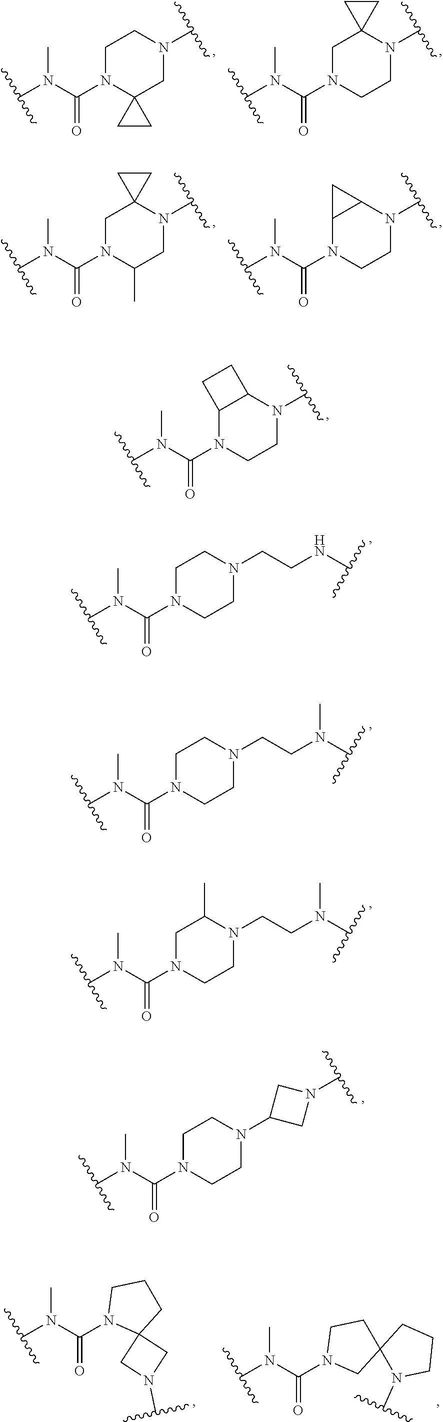

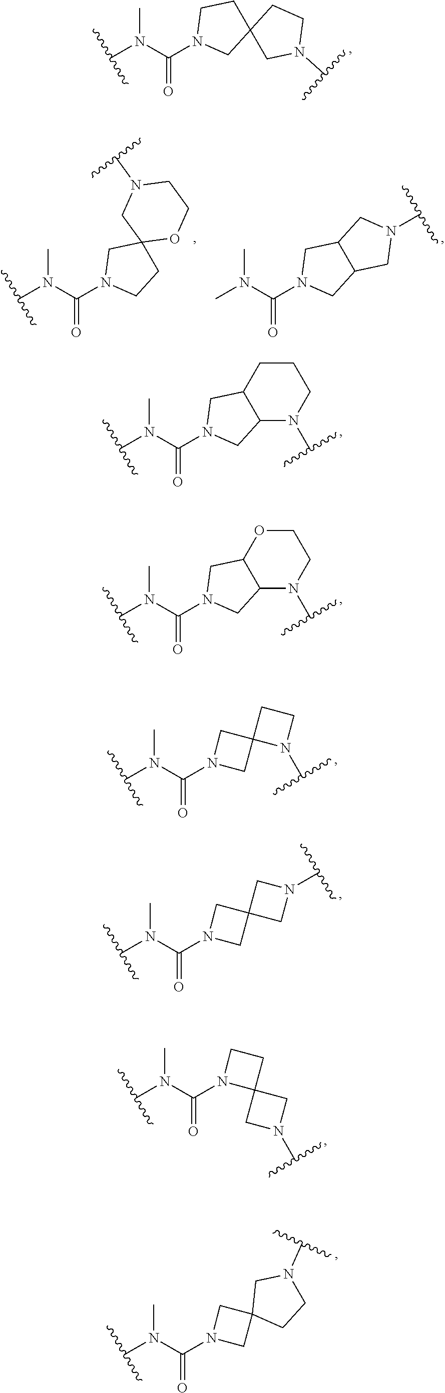

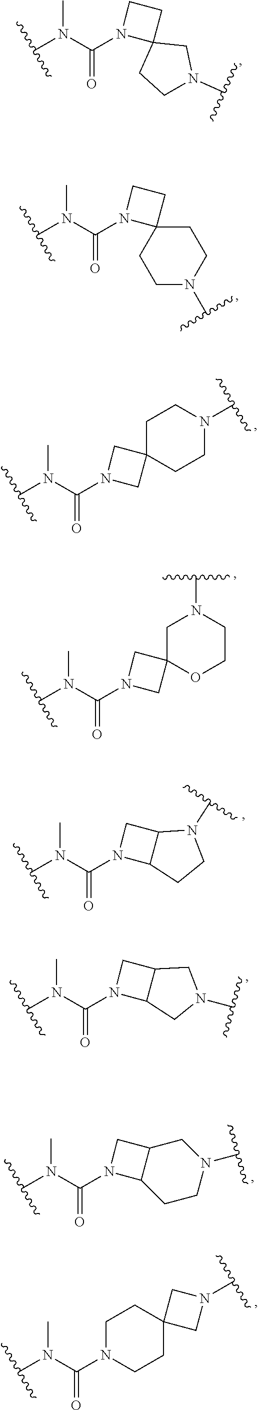

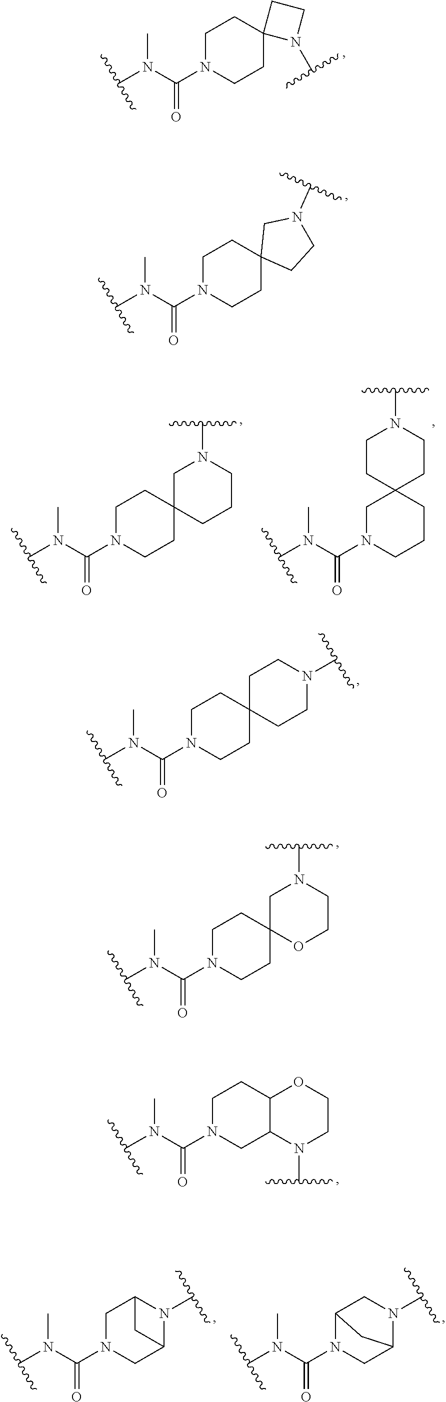

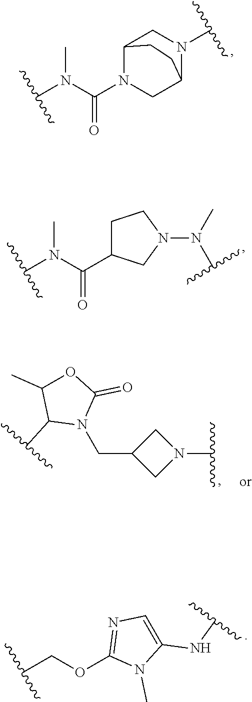 [SMILES: *N(C)C(=O)N1CC2CCC1CN2* ; *N(C)C(=O)C1CCN(N(*)C)C1 ; *C1C(C)OC(=O)N1CC1CN(*)C1 ; *COc1ncc(N*)n1C]

In some embodiments, the linker has the structure

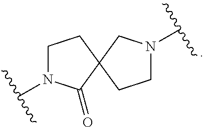 [SMILES: *N1CCC2(CCN(*)C2=O)C1]

In some embodiments, the linker has the structure

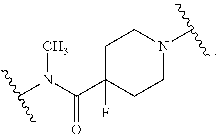 [SMILES: *N1CCC(F)(C(=O)N(*)C)CC1]

In some embodiments of a compound of the present invention, W is a cross-linking group comprising a vinyl ketone. In some embodiments, W has the structure of Formula IIIa:

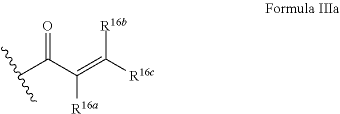

wherein R^{16a}, R^{16b}, and R^{16c} are, independently, hydrogen, —CN, halogen, or —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl. In some embodiments, W is:

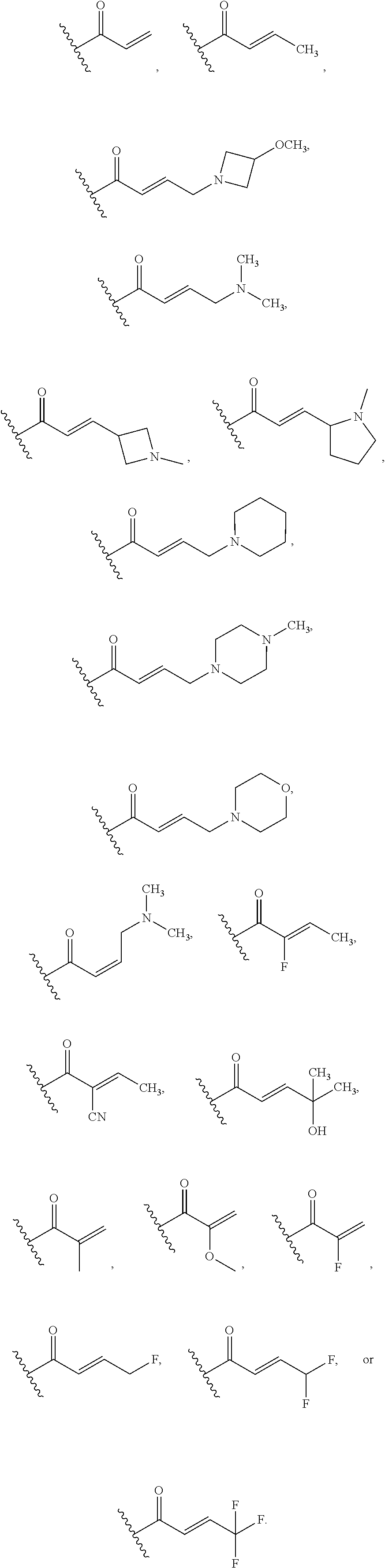

In some embodiments, W is a cross-linking group comprising an ynone. In some embodiments, W has the structure of Formula IIIb:

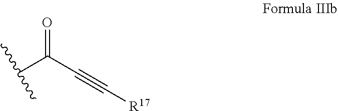

wherein R¹⁷ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl, or a 4 to 7-membered saturated heterocycloalkyl. In some embodiments, W is:

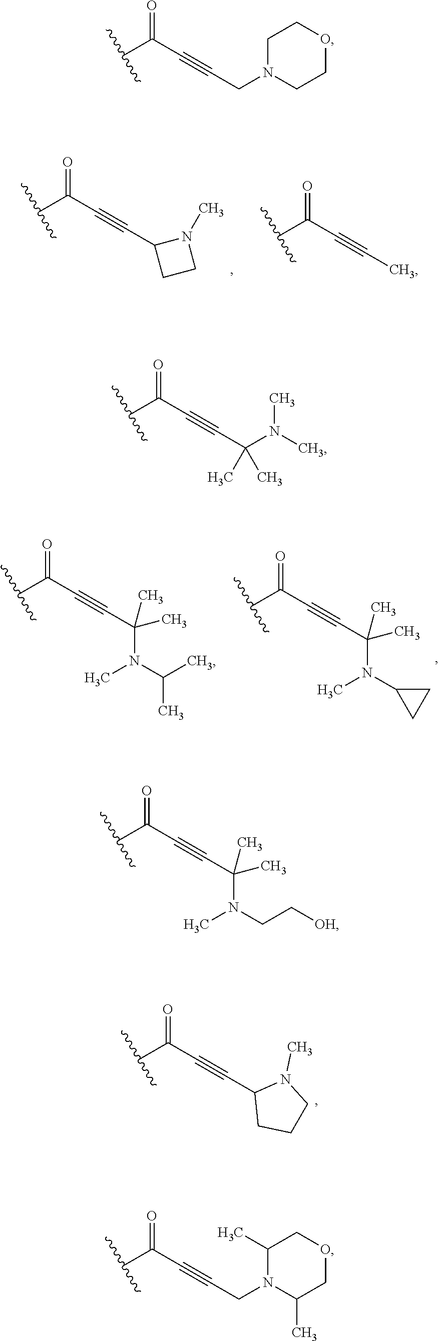

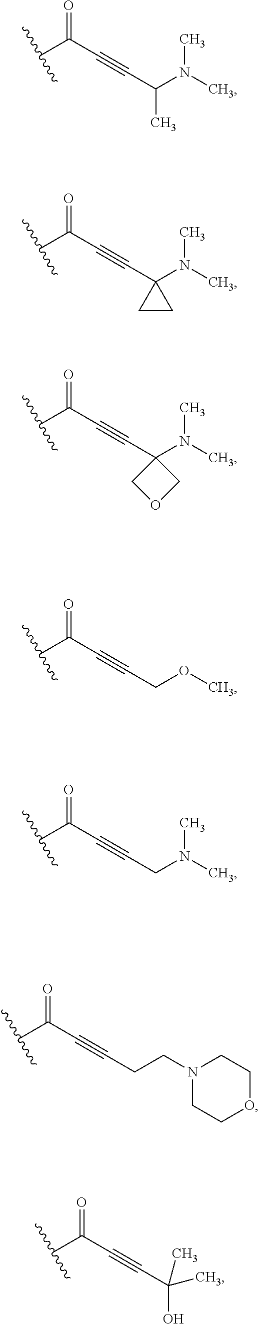

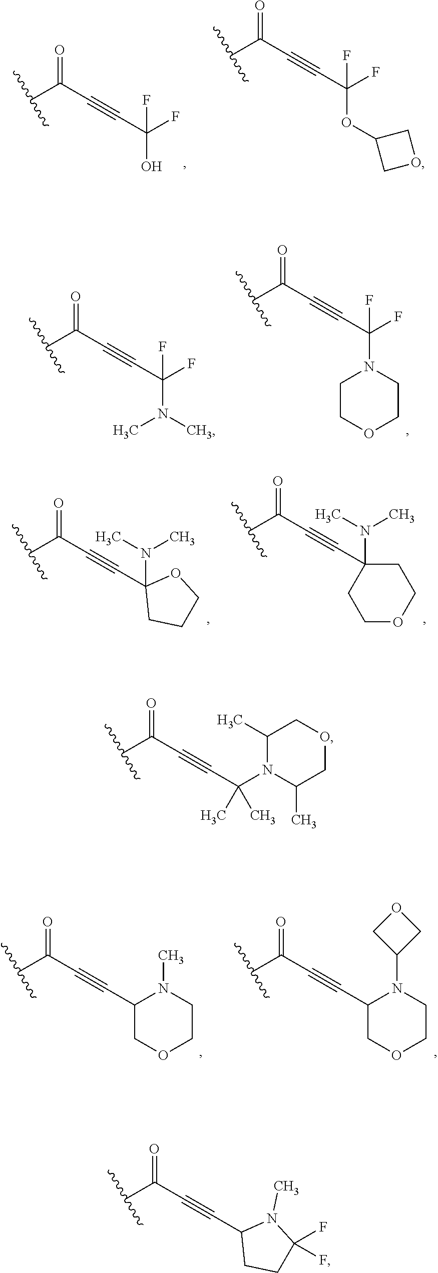

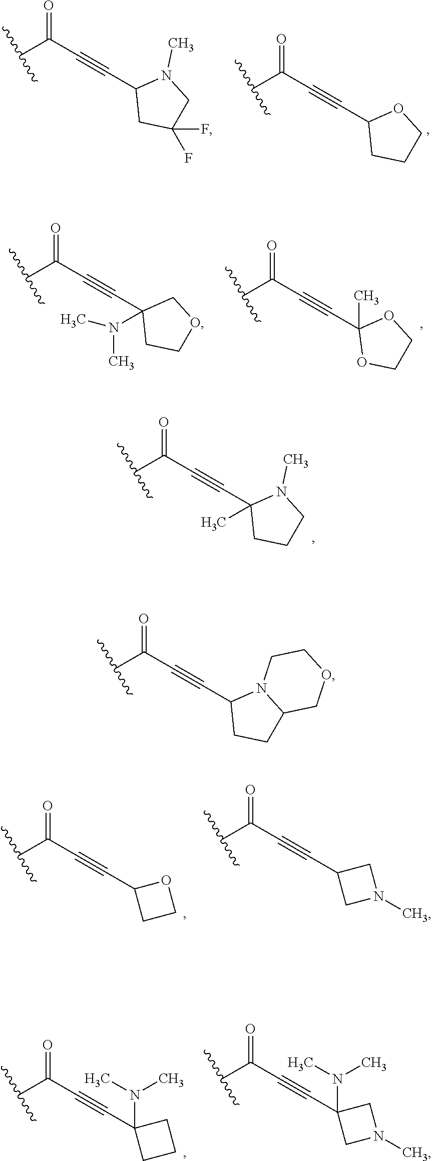

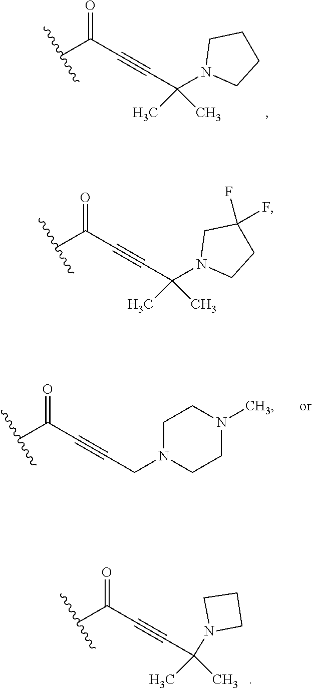 [SMILES: *C(=O)C#CC(C)(C)N1CCCC1 ; *C(=O)C#CC(C)(C)N1CCC(F)(F)C1 ; *C(=O)C#CCN1CCN(C)CC1 ; *C(=O)C#CC(C)(C)N1CCC1]

In some embodiments, W is

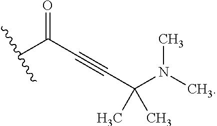 [SMILES: *C(=O)C#CC(C)(C)N(C)C]

In some embodiments, W is a cross-linking group comprising a vinyl sulfone. In some embodiments, W has the structure of Formula IIIc:

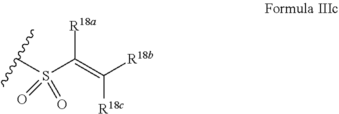

wherein R^{18a}, R^{18b}, and R^{18c} are, independently, hydrogen, —CN, or —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl. In some embodiments, W is:

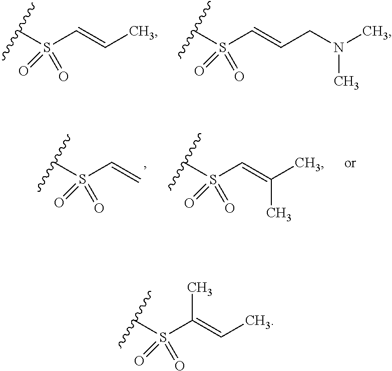

In some embodiments, W is a cross-linking group comprising an alkynyl sulfone. In some embodiments, W has the structure of Formula IIId:

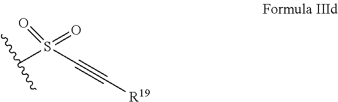

wherein R¹⁹ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl, or a 4 to 7-membered saturated heterocycloalkyl. In some embodiments, W is:

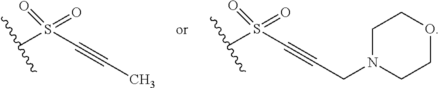

In some embodiments, W has the structure of Formula Ille:

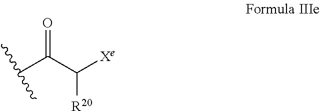

wherein X^{e} is a halogen; and
R²⁰ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl. In some embodiments, W is haloacetal. In some embodiments, W is not haloacetal.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 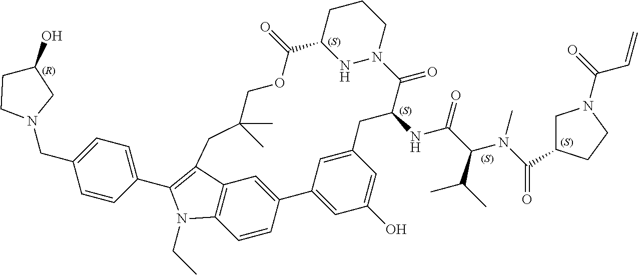 |
| A2 | 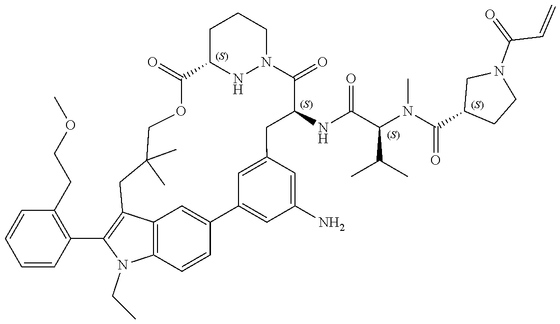 |
| A3 | 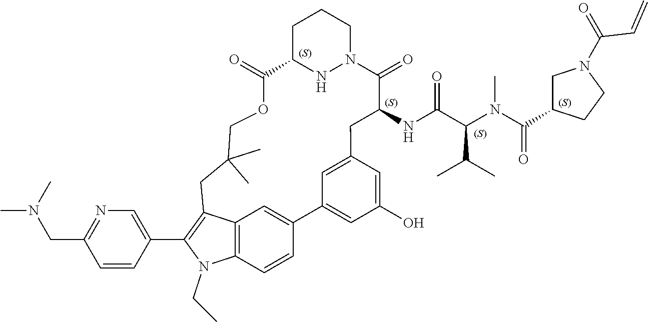 |
| A4 | 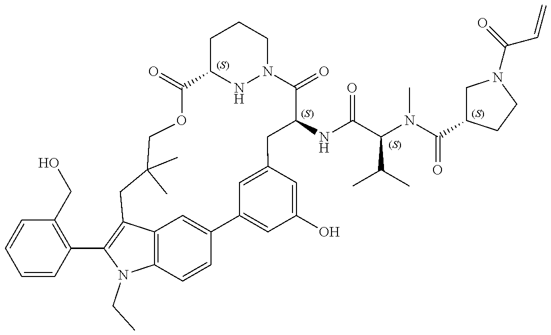 |
| A5 | 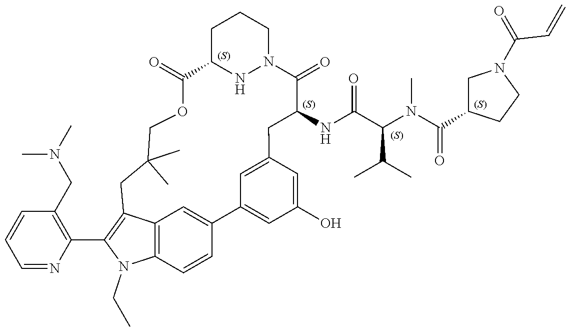 |
| A6 | 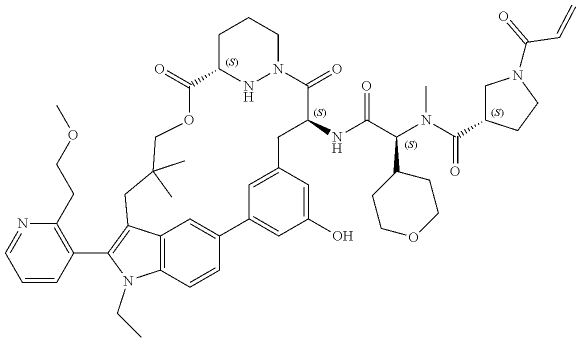 |
| A7 | 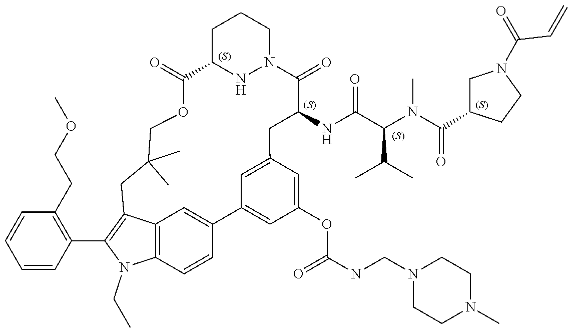 |
| A8 | 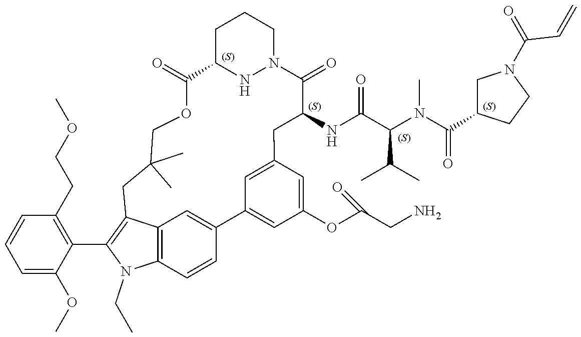 |
| A9 | 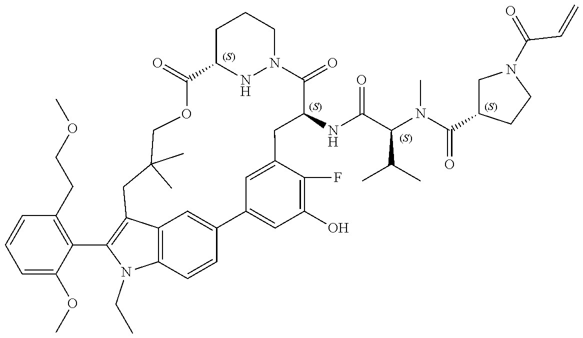 |
| A10 | 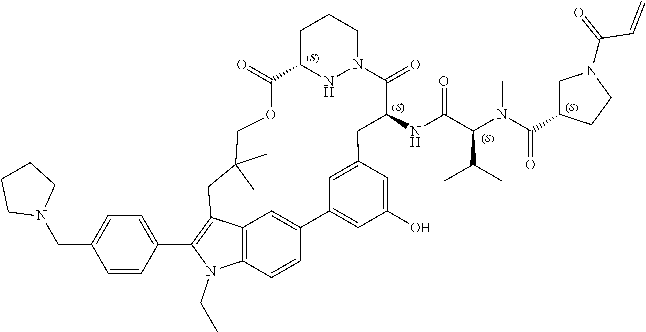 |
| A11 | 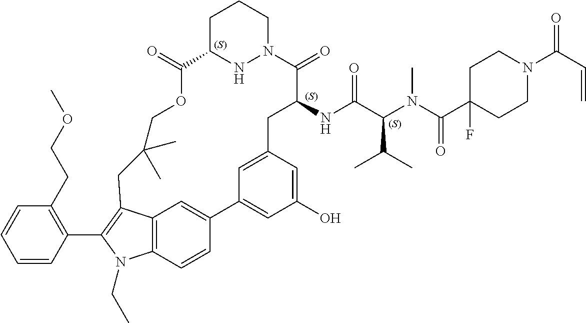 |
| A12 | 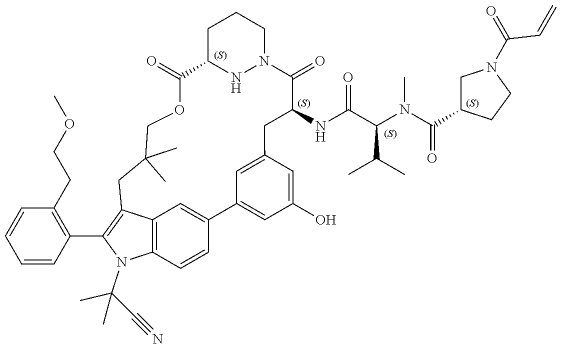 |
| A13 | 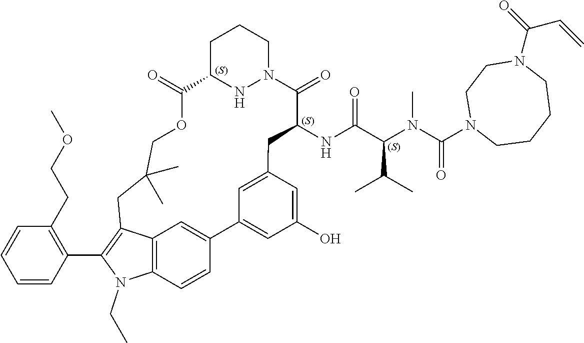 |
| A14 | 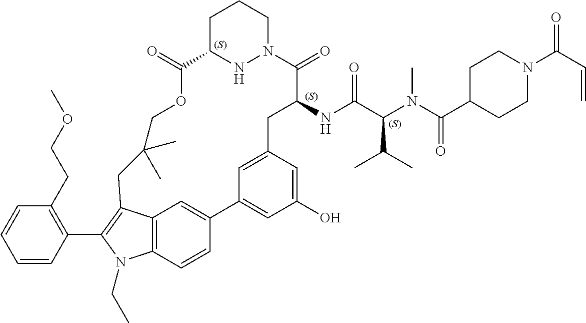 |
| A15 | 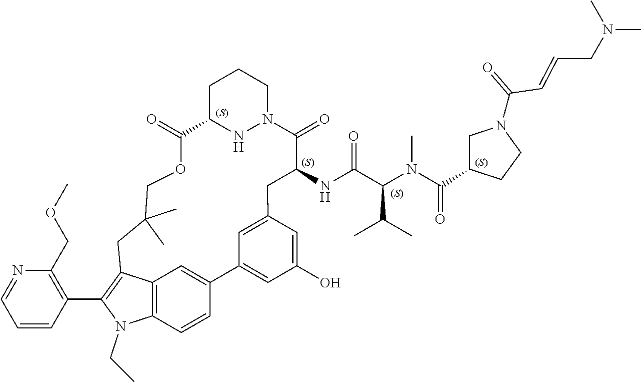 |
| A16 | 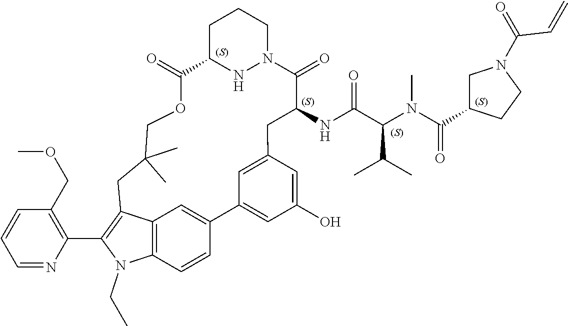 |
| A17 | 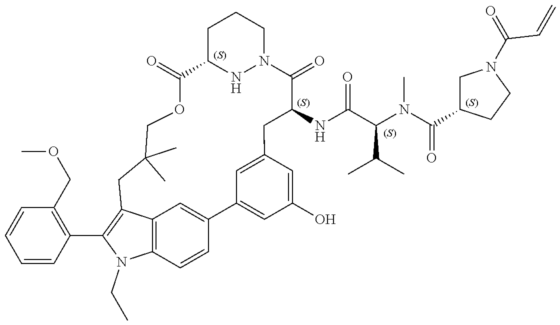 |
| A18 | 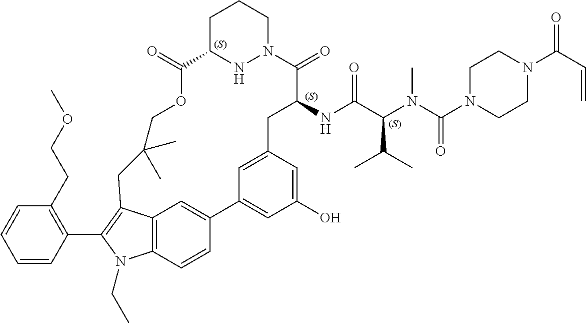 |
| A19 | 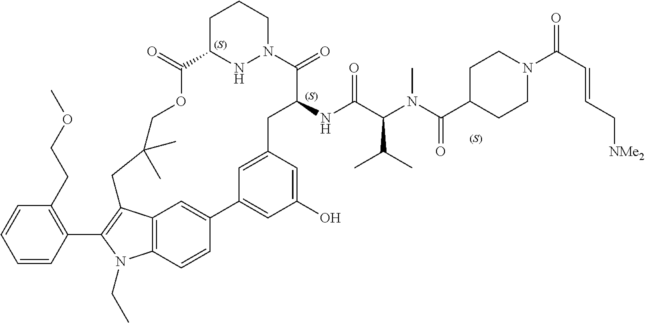 |
| A20 | 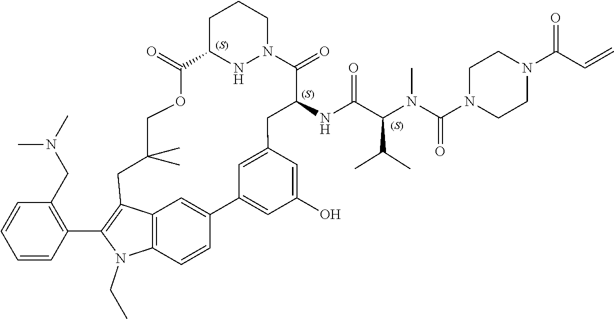 |
| A21 | 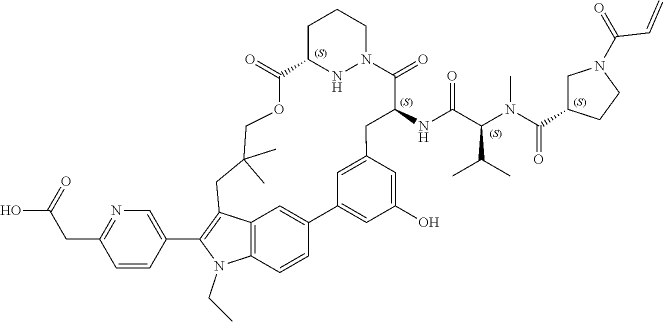 |
| A22 | 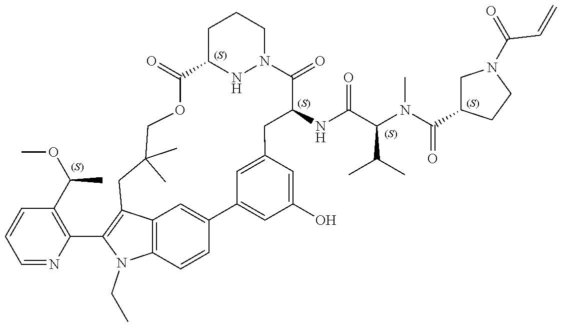 |
| A23 | 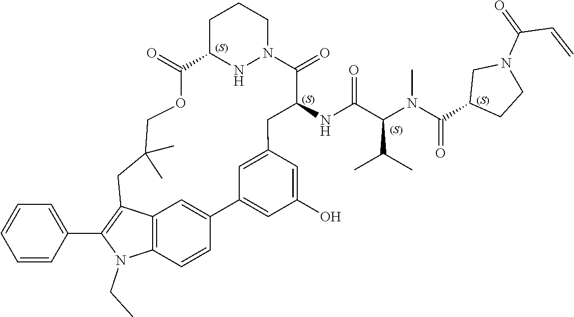 |
| A24 | 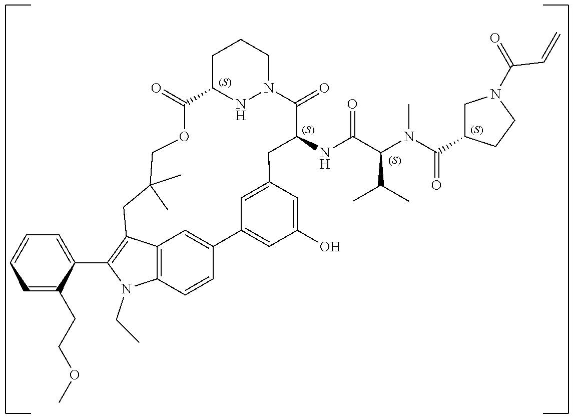 |
| A25 | 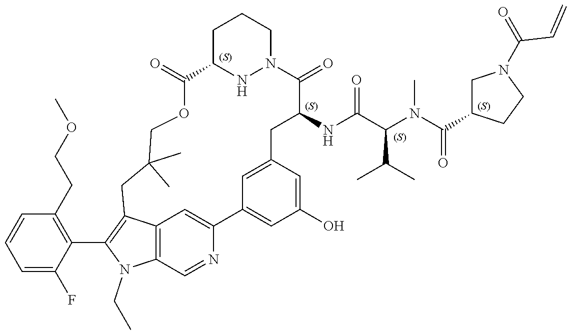 |
| A26 | 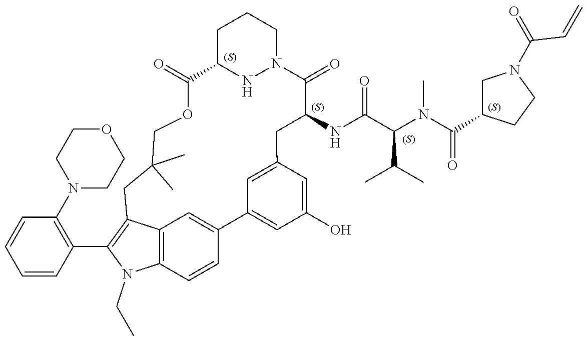 |
| A27 | 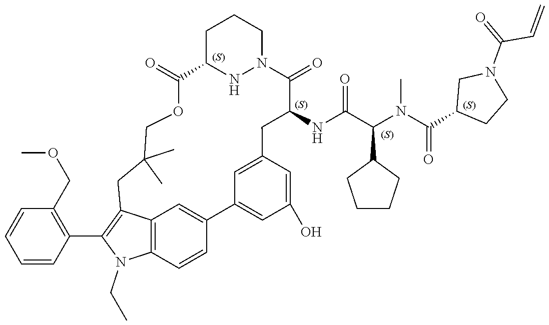 |
| A28 | 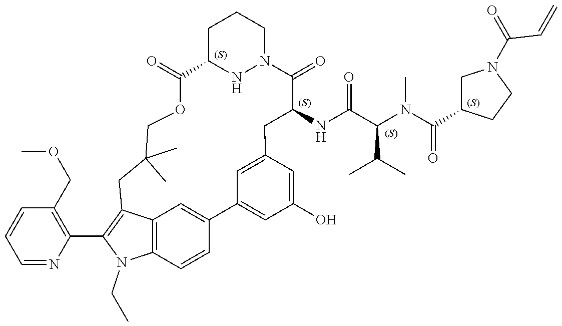 |
| A29 | 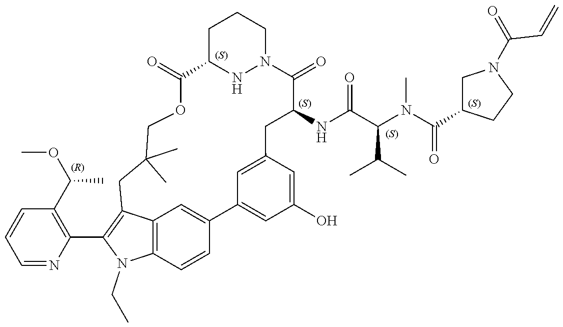 |
| A30 | 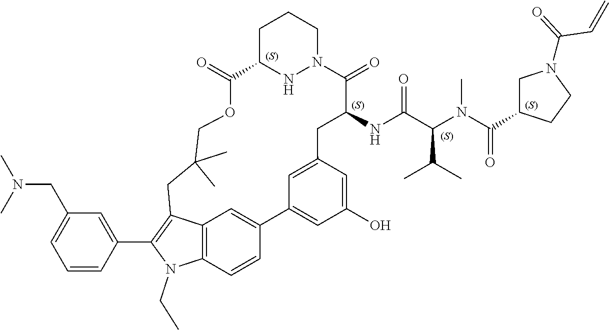 |
| A31 | 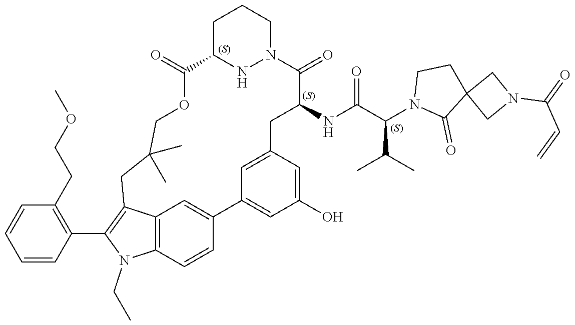 |
| A32 | 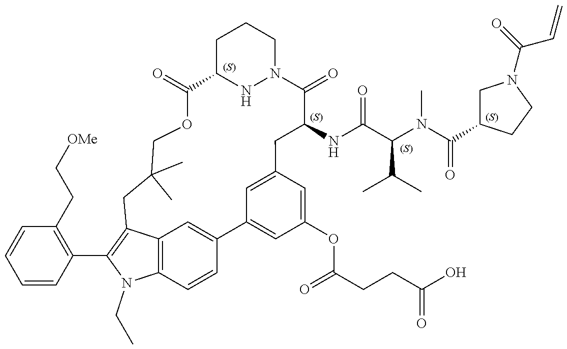 |
| A33 | 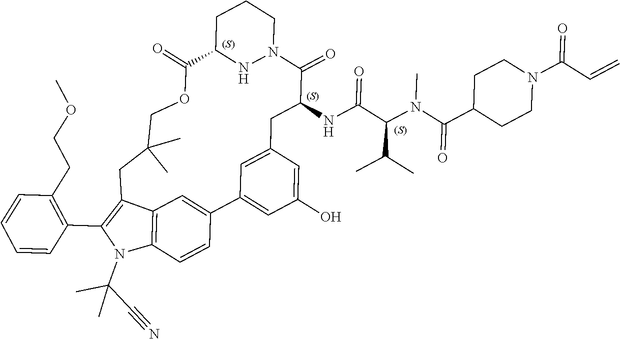 |
| A34 | 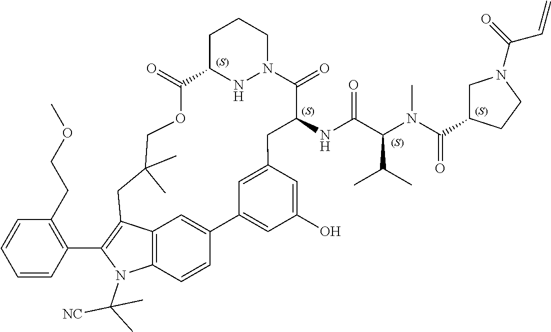 |
| A35 | 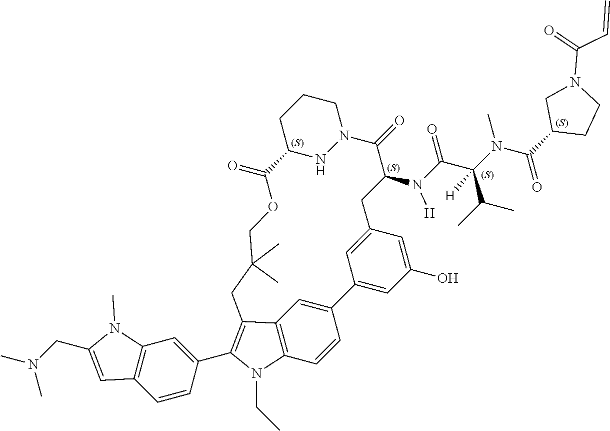 |
| A36 | 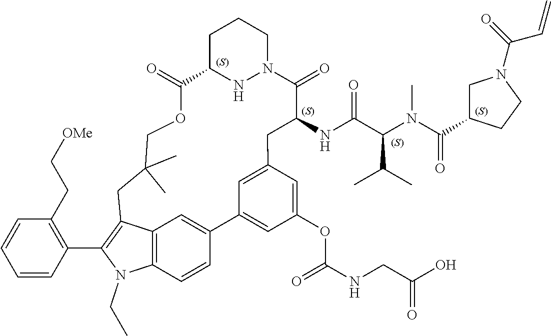 |
| A37 | 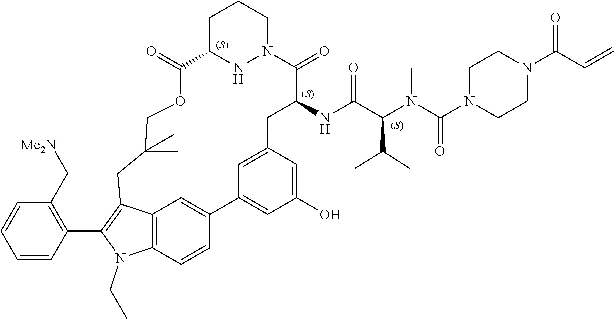 |
| A38 | 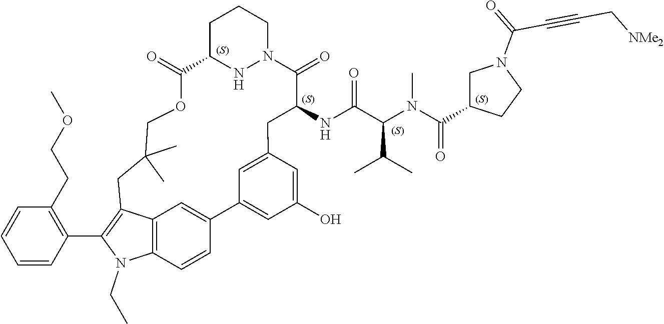 |
| A39 | 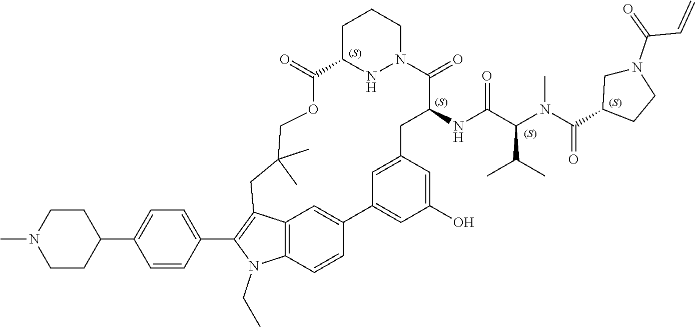 |
| A40 | 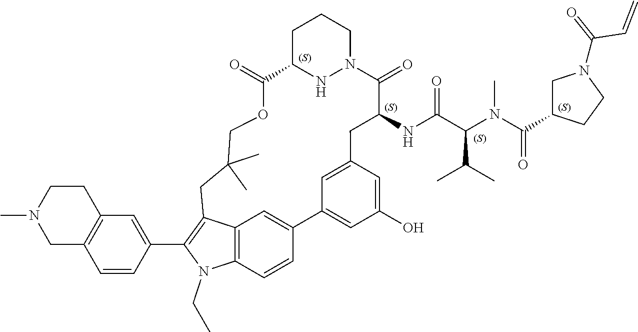 |
| A41 | 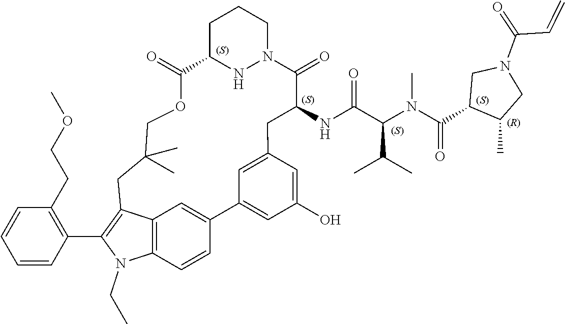 |
| A42 | 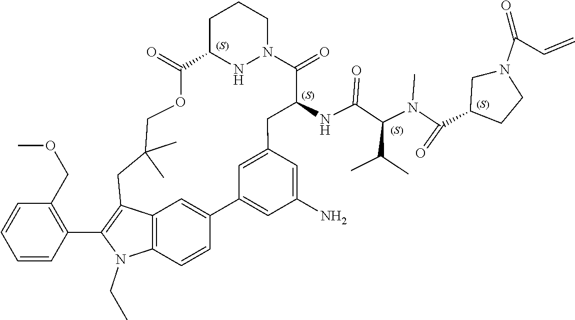 |
| A43 | 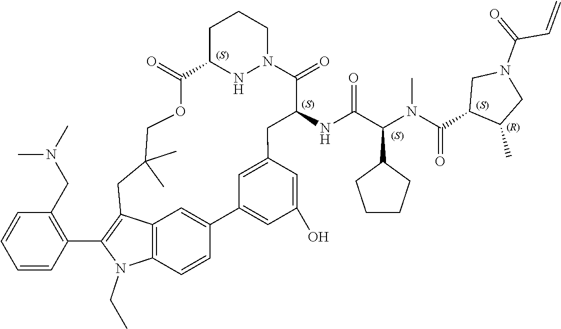 |
| A44 | 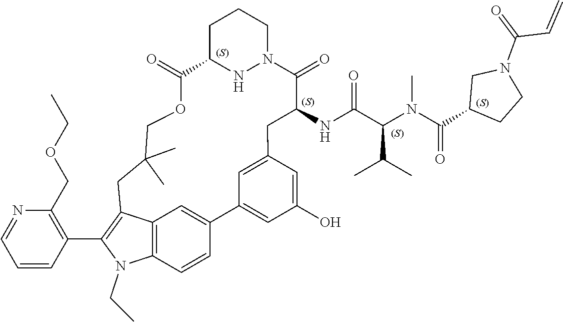 |
| A45 | 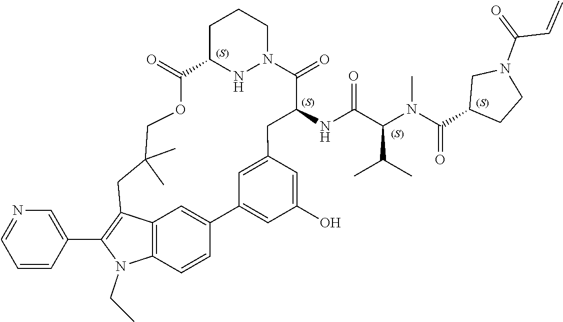 |
| A46 | 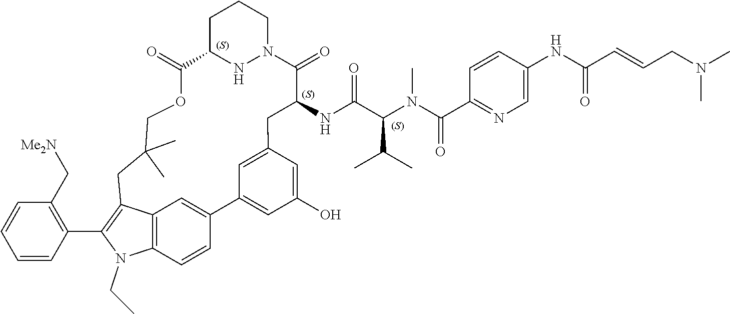 |
| A47 | 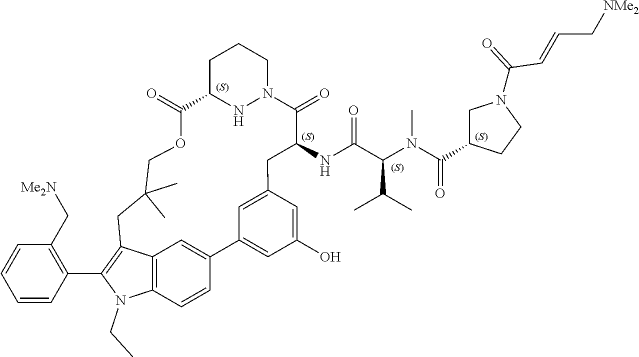 |
| A48 | 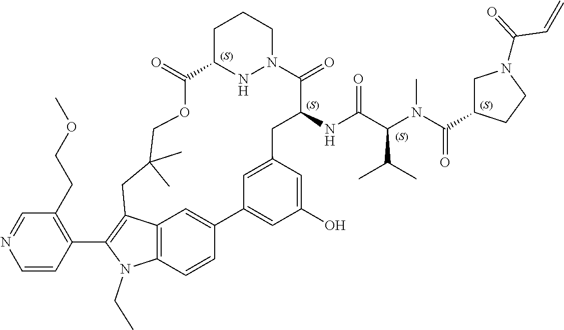 |
| A49 | 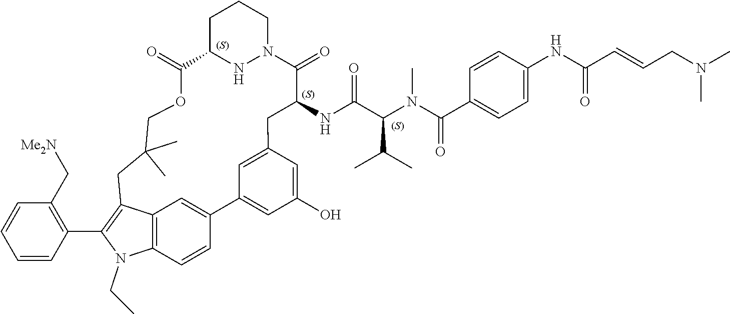 |
| A50 | 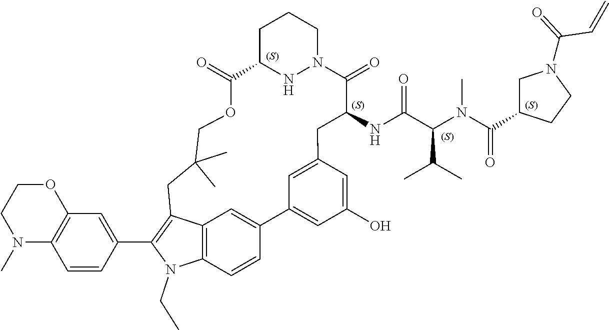 |
| A51 | 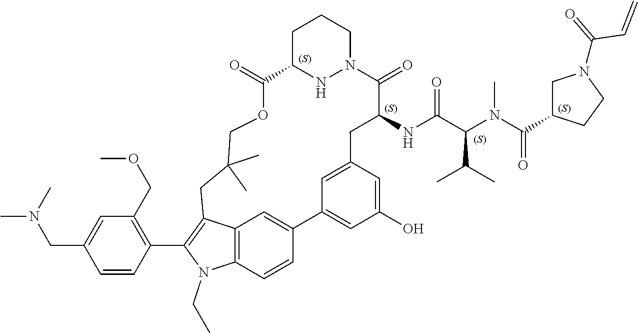 |
| A52 | 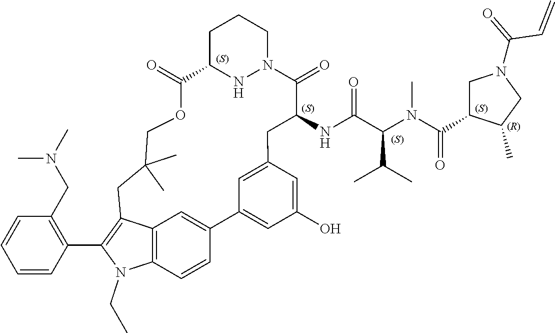 |
| A53 | 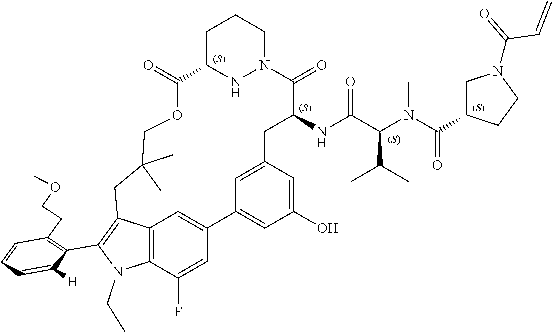 |
| A54 | 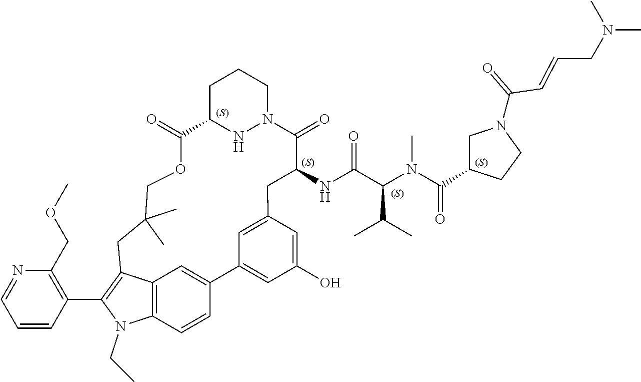 |
| A55 | 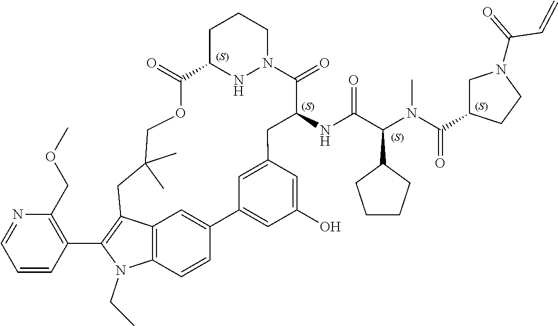 |
| A56 | 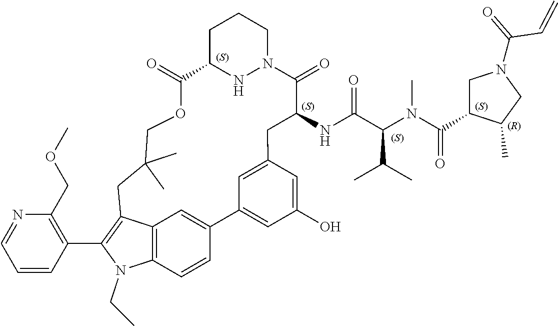 |
| A57 | 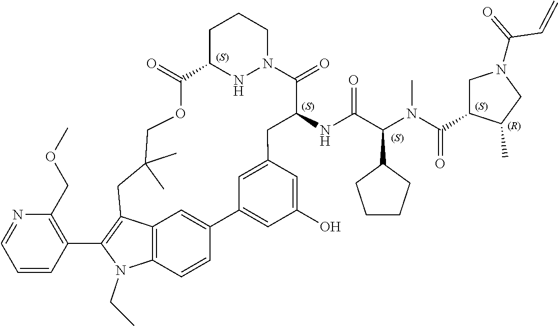 |
| A58 | 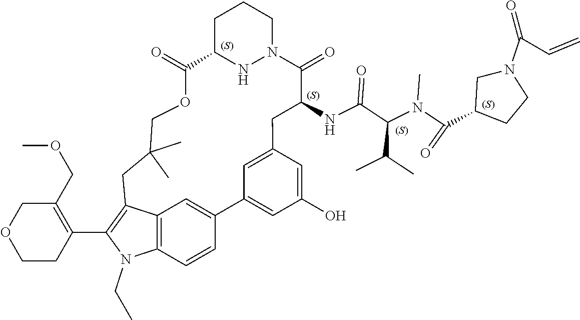 |
| A59 | 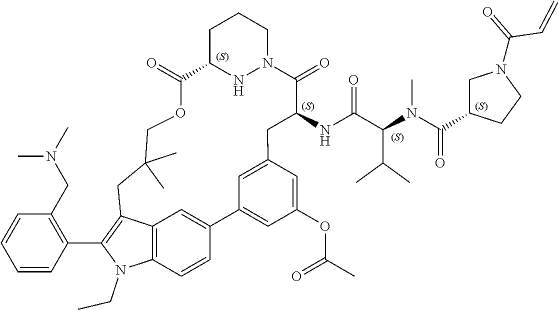 |
| A60 | 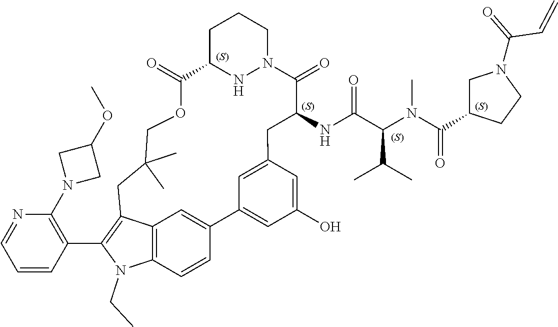 |
| A61 | 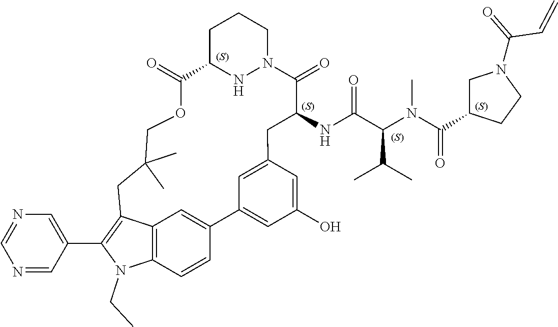 |
| A62 | 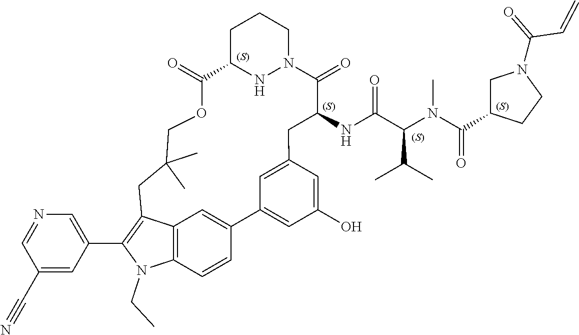 |
| A63 | 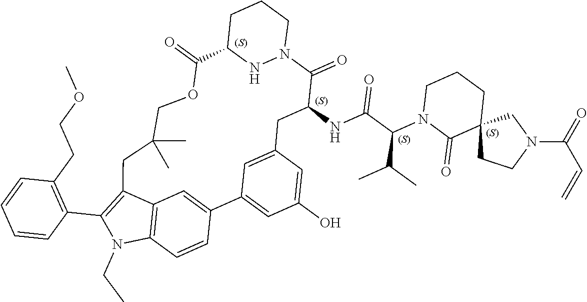 |
| A64 | 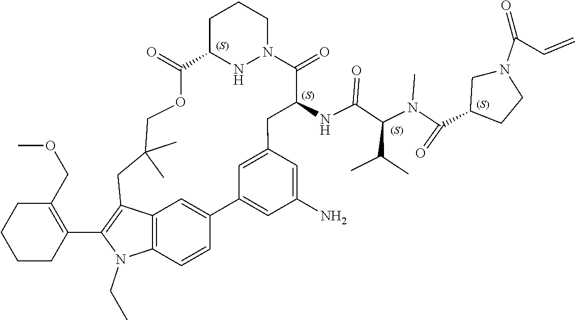 |
| A65 | 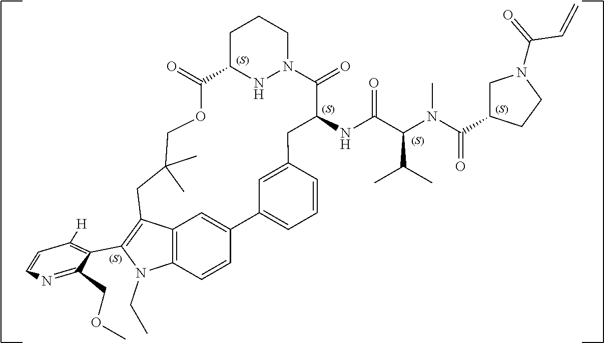 |
| A66 | 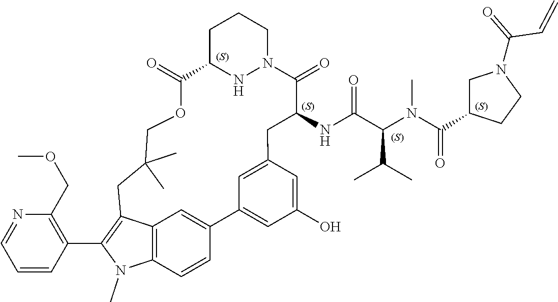 |
| A67 | 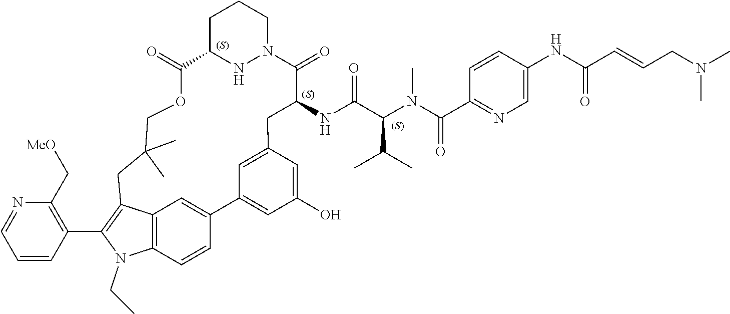 |
| A68 | 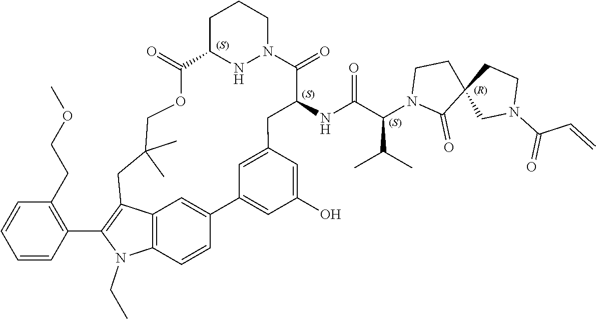 |
| A69 | 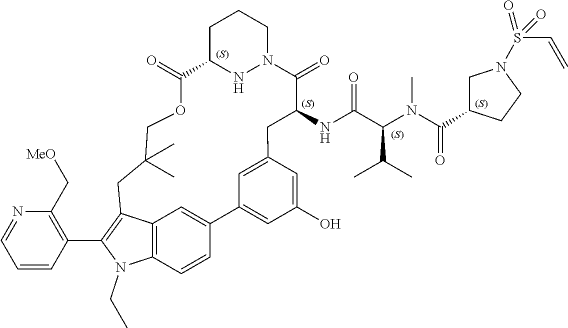 |
| A70 | 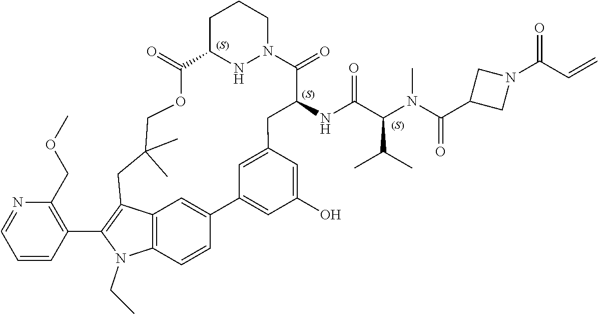 |
| A71 | 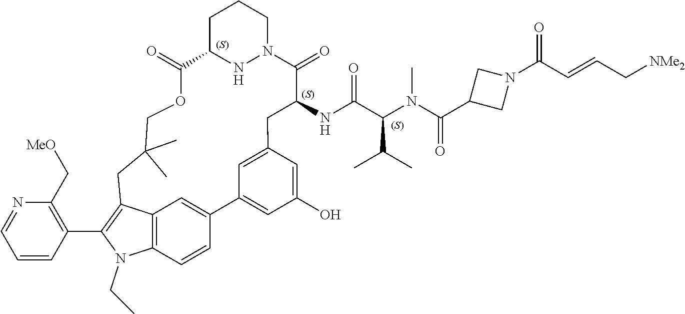 |
| A72 | 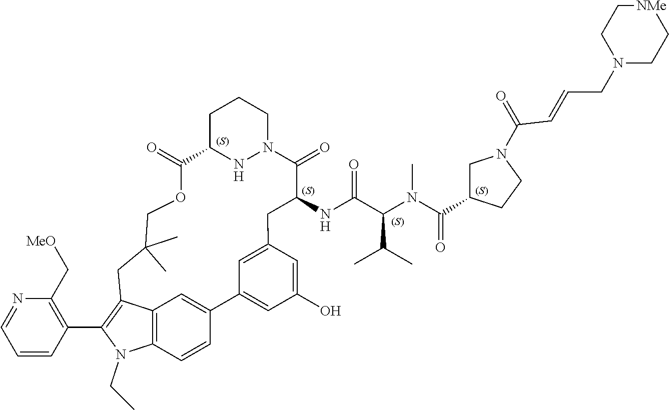 |
| A73 | 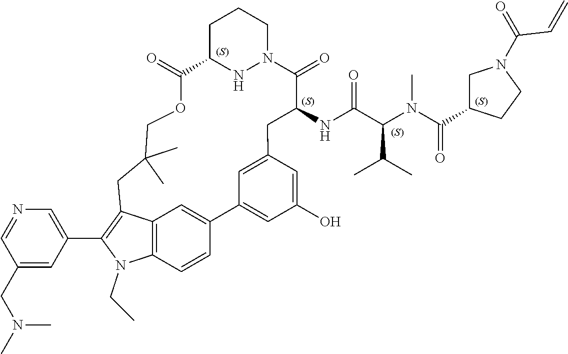 |
| A74 | 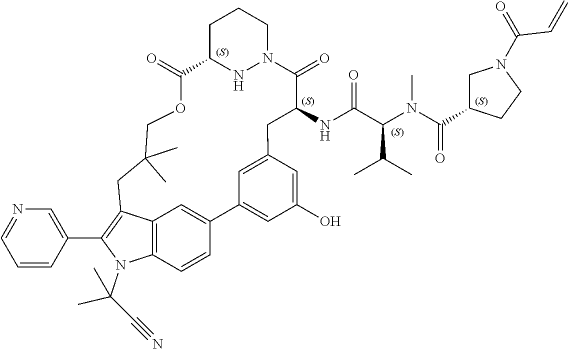 |
| A75 | 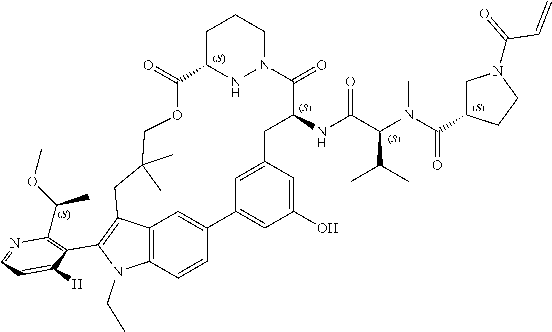 |
| A76 | 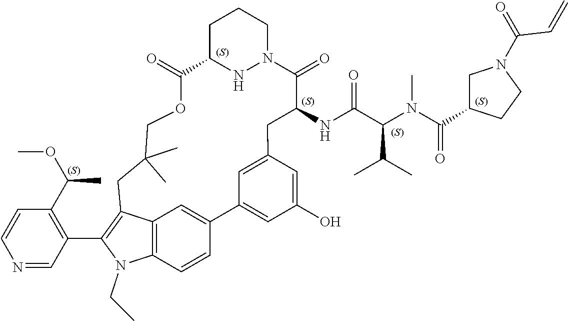 |
| A77 | 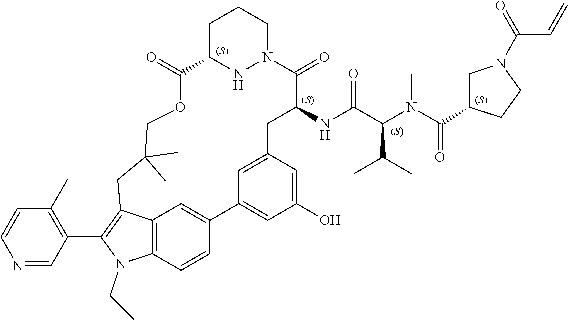 |
| A78 | 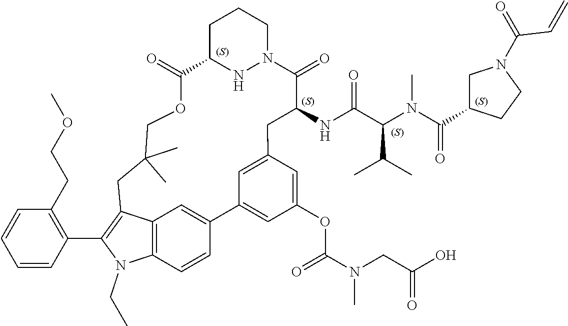 |
| A79 | 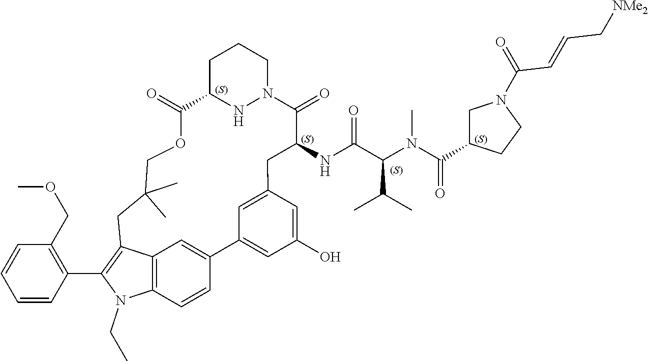 |
| A80 | 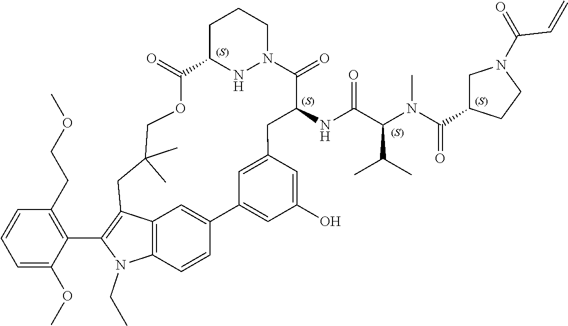 |
| A81 | 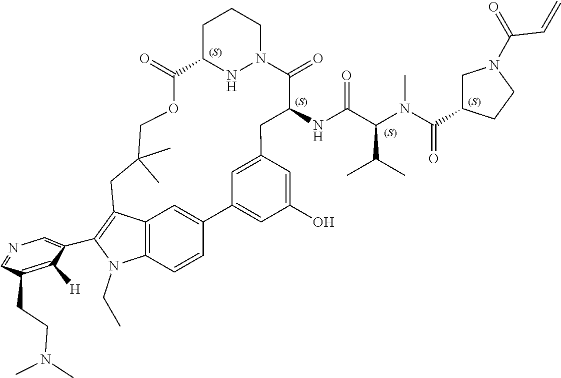 |
| A82 | 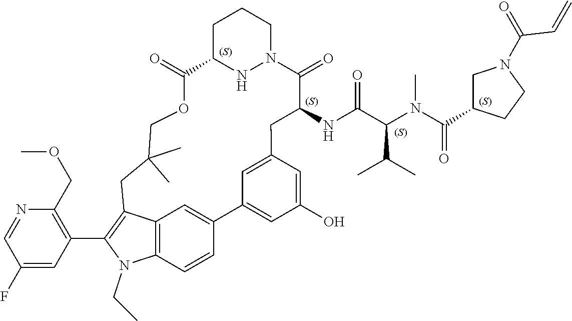 |
| A83 | 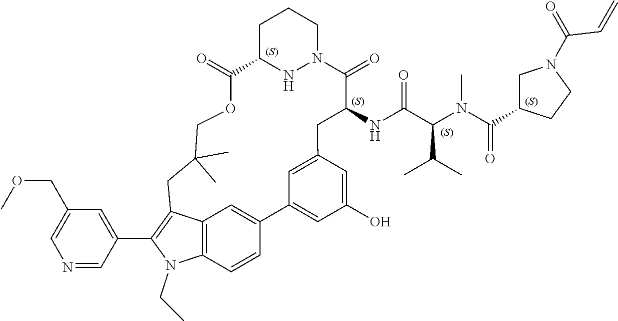 |
| A84 | 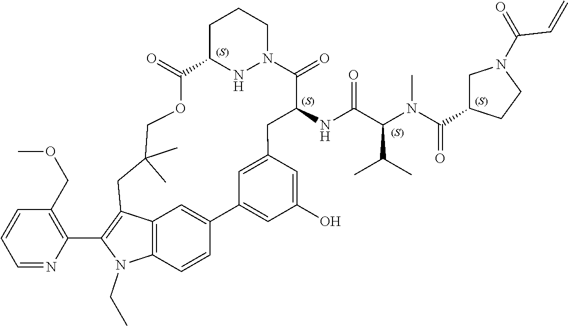 |
| A85 | 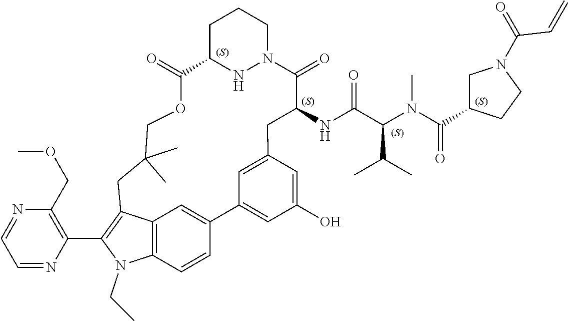 |
| A86 | 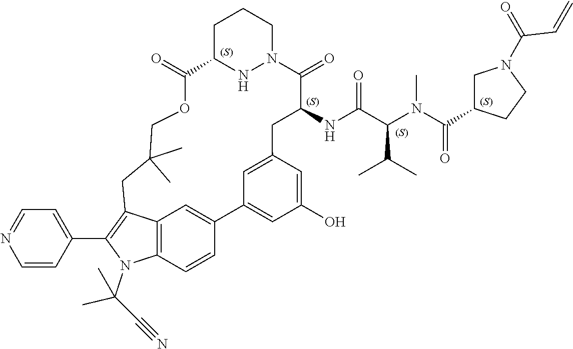 |
| A87 | 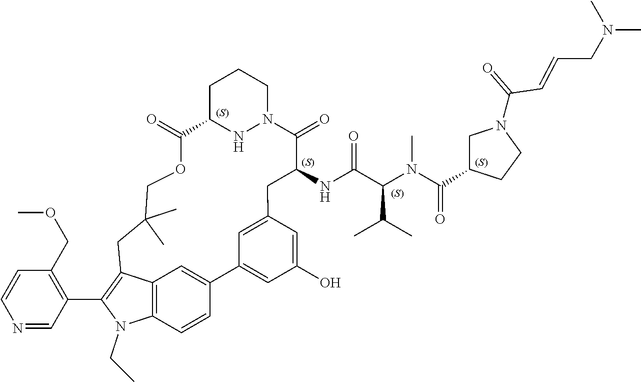 |
| A88 | 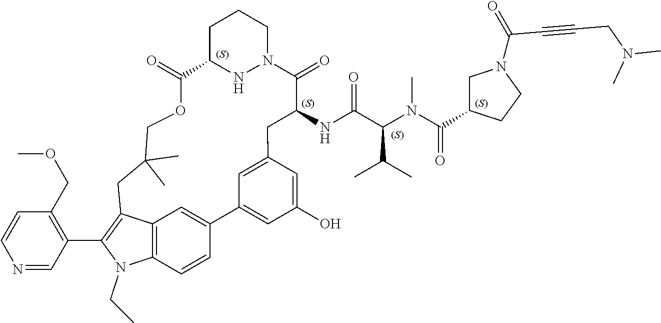 |
| A89 | 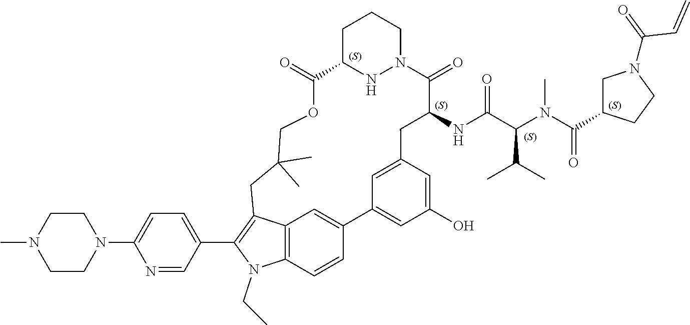 |
| A90 | 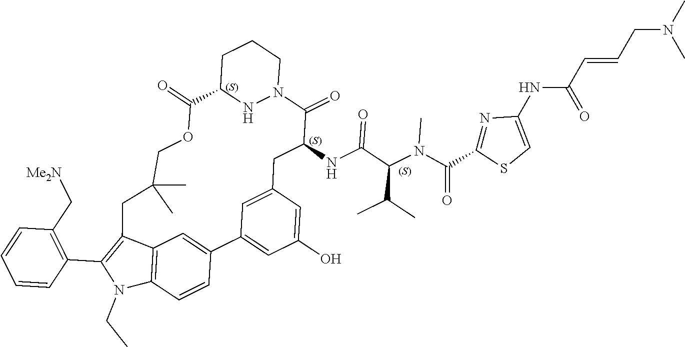 |
| A91 | 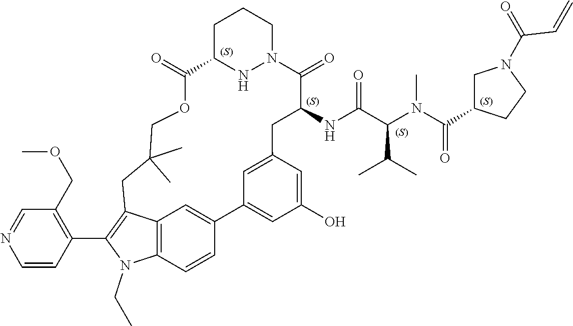 |
| A92 | 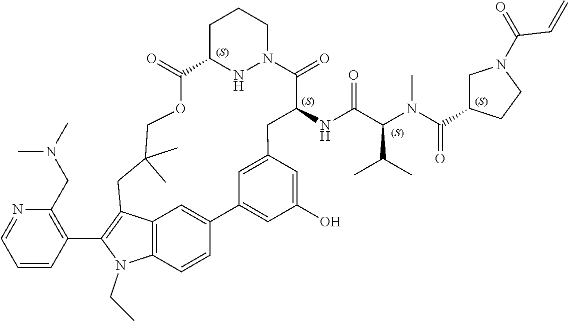 |
| A93 | 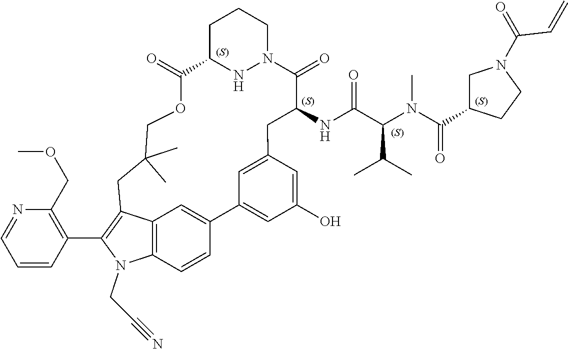 |
| A94 | 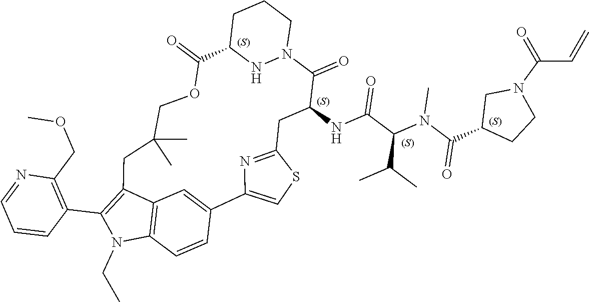 |
| A95 | 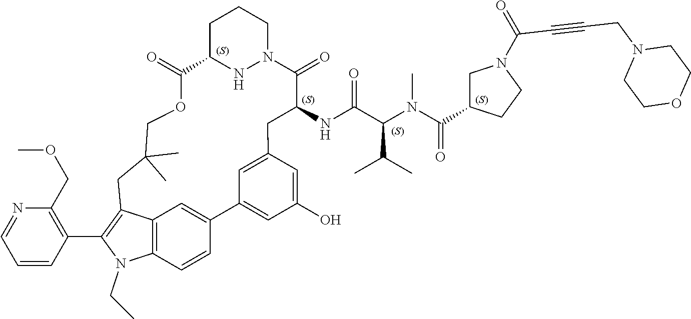 |
| A96 | 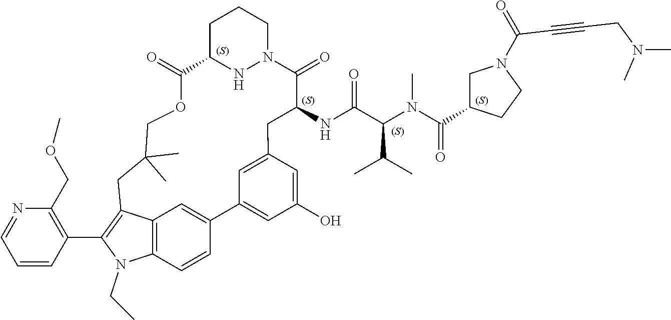 |
| A97 | 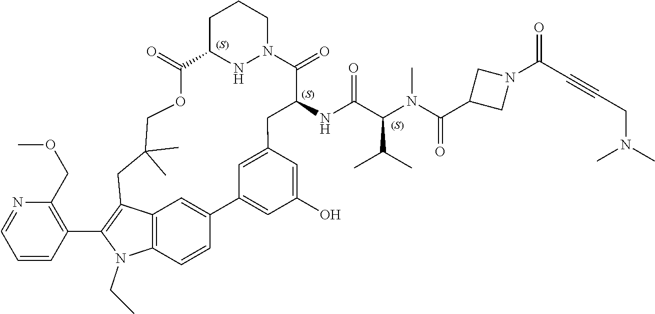 |
| A98 | 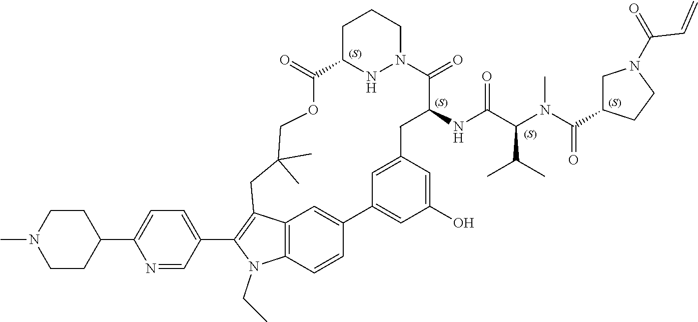 |
| A99 | 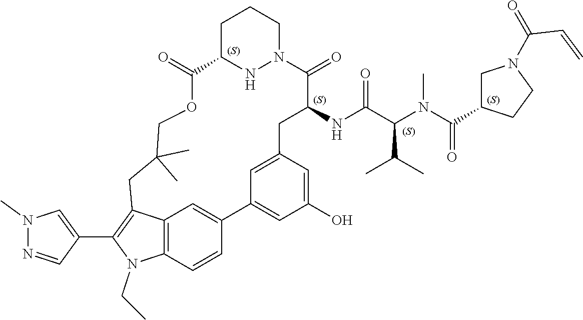 |
| A100 | 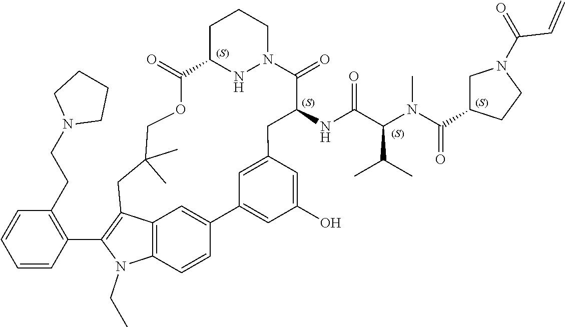 |
| A101 | 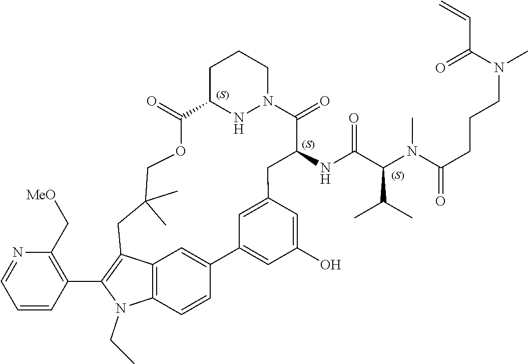 |
| A102 | 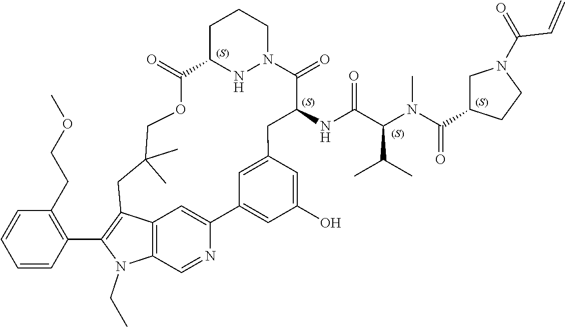 |
| A103 | 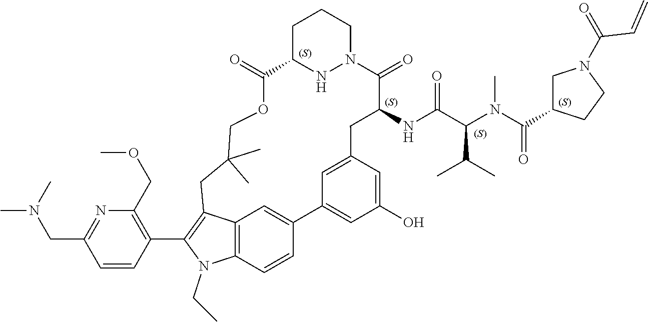 |
| A104 | 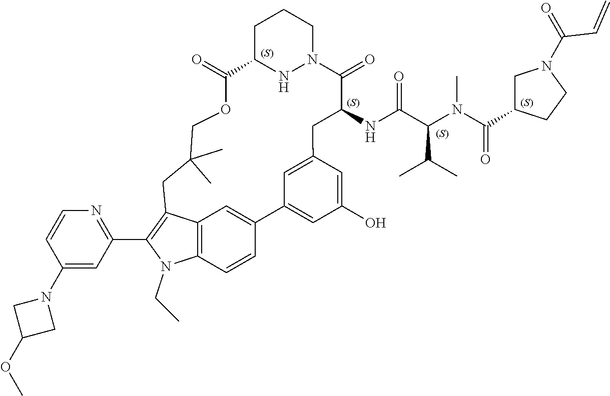 |
| A105 | 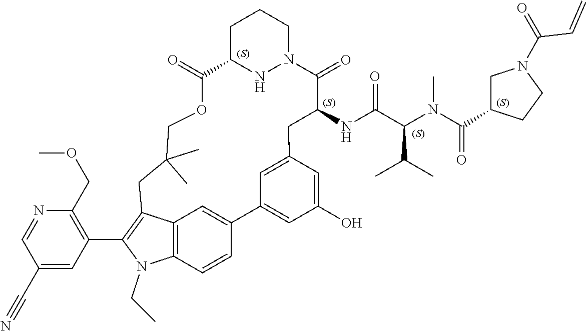 |
| A106 | 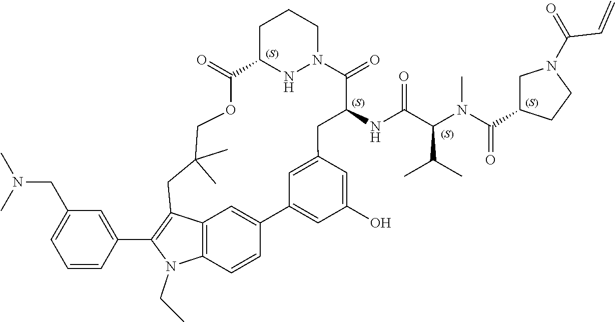 |
| A107 | 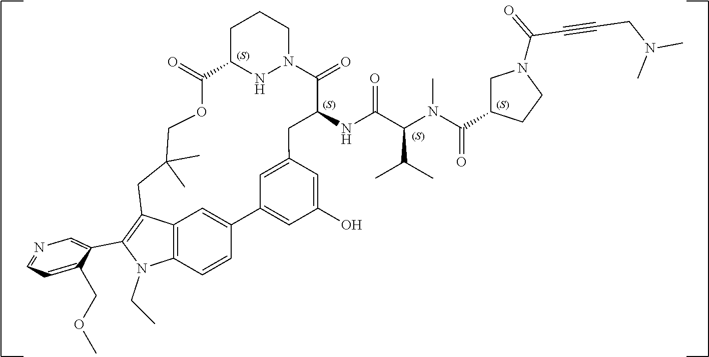 |
| A108 | 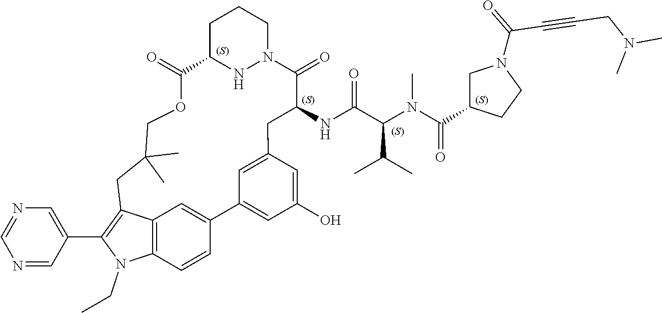 |
| A109 | 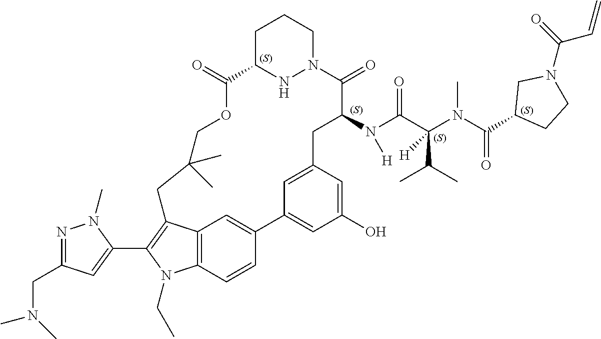 |
| A110 | 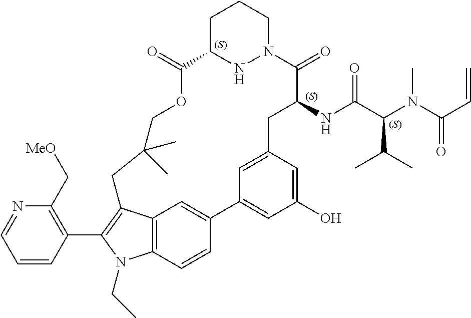 |
| A111 | 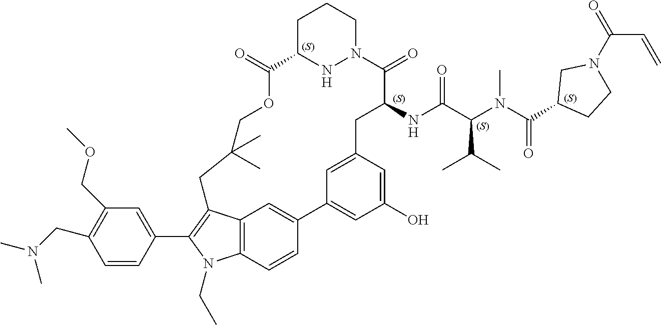 |
| A112 | 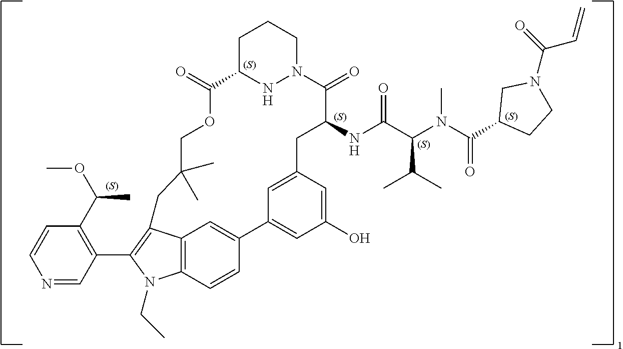 |
| A113 | 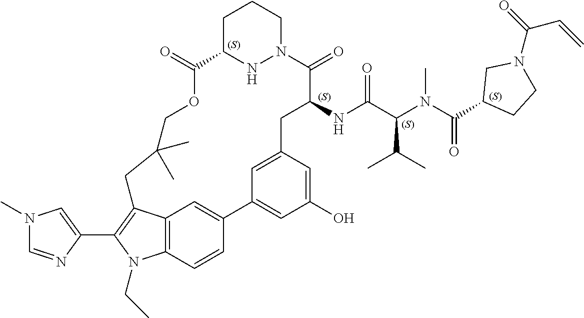 |
| A114 | 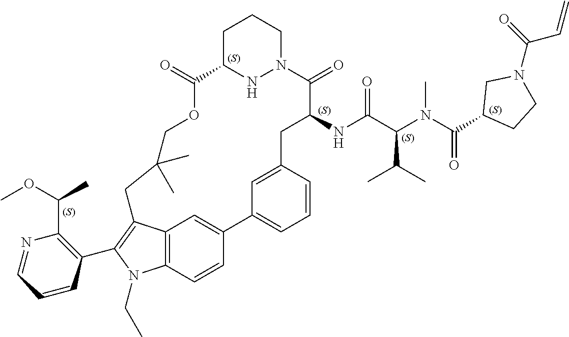 |
| A115 | 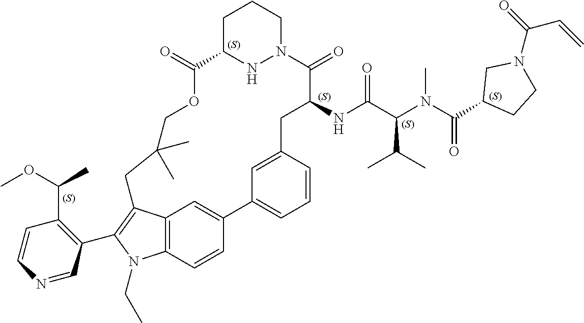 |
| A116 | 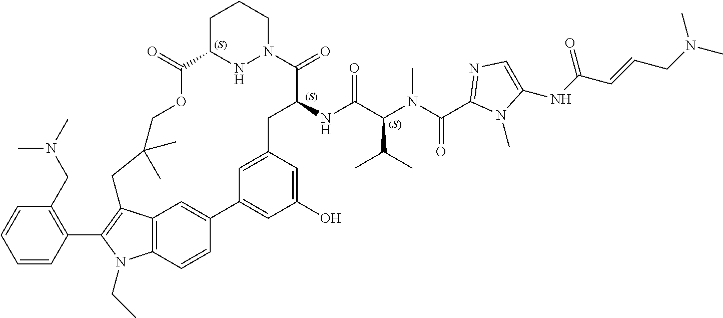 |
| A117 | 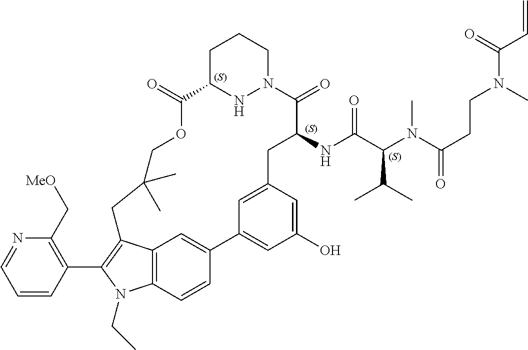 |
| A118 | 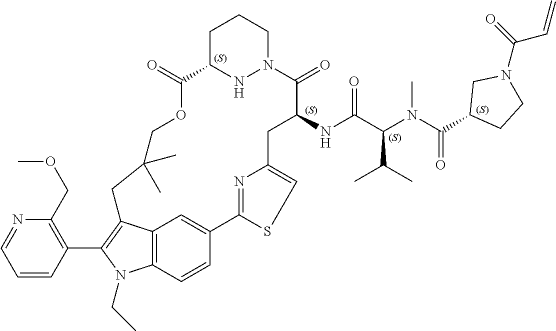 |
| A119 | 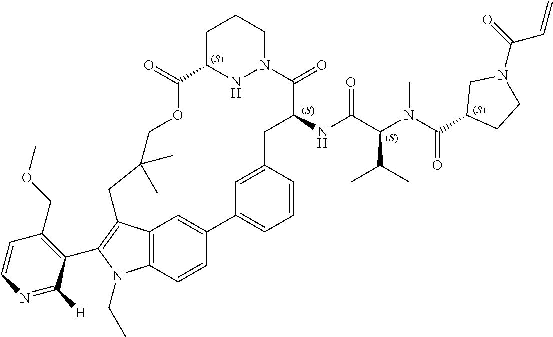 |
| A120 | 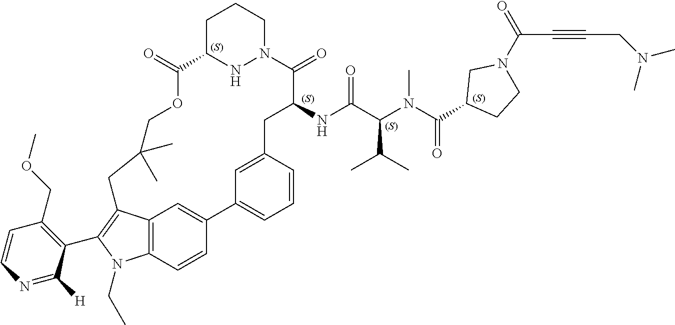 |
| A121 | 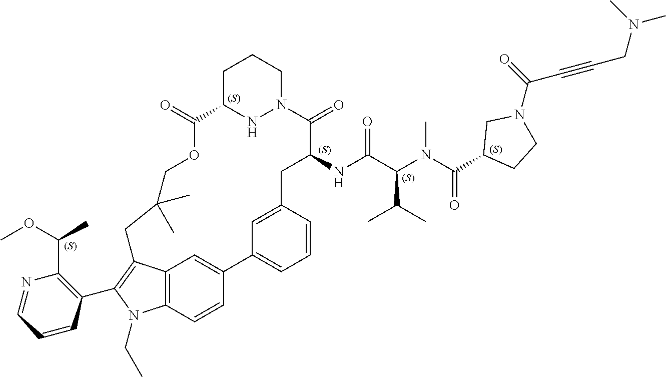 |
| A122 | 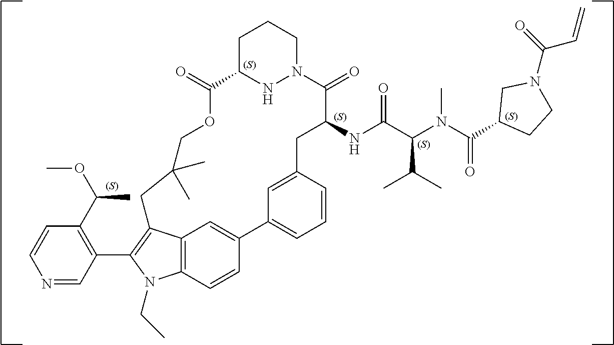 |
| A123 | 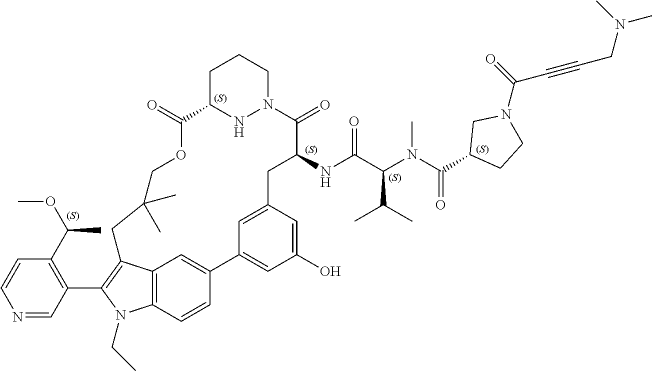 |
| A124 | 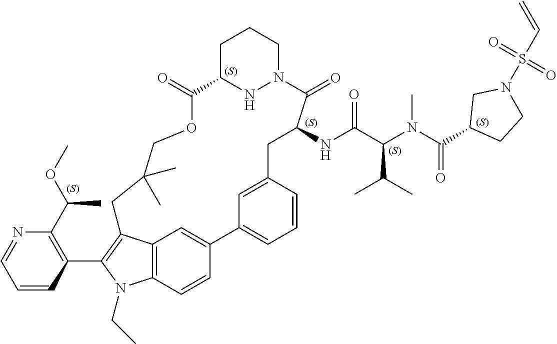 |
| A125 | 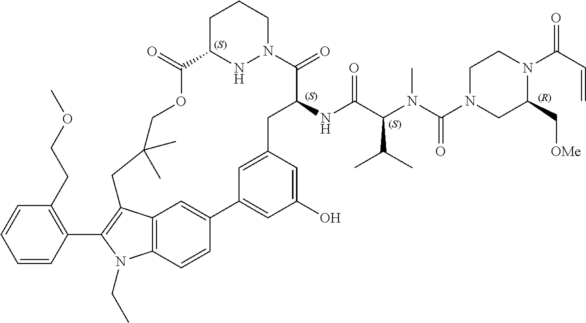 |
| A126 | 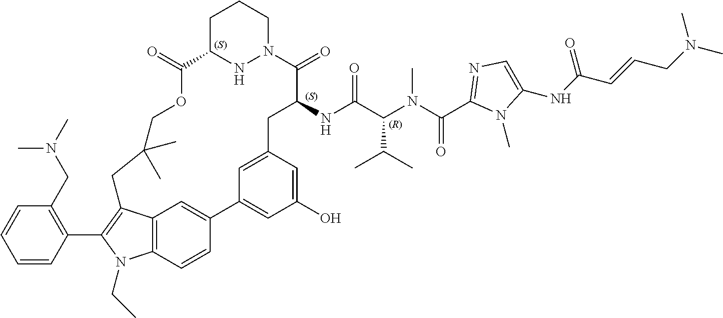 |
| A127 | 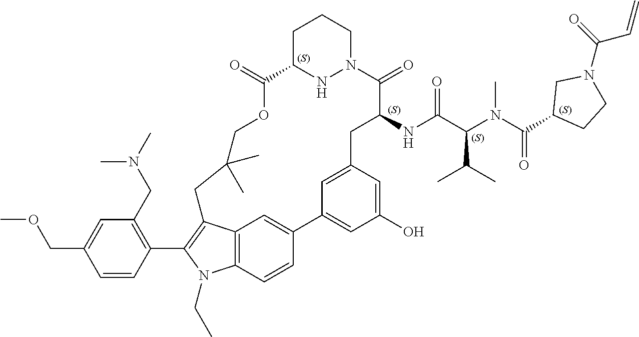 |
| A128 | 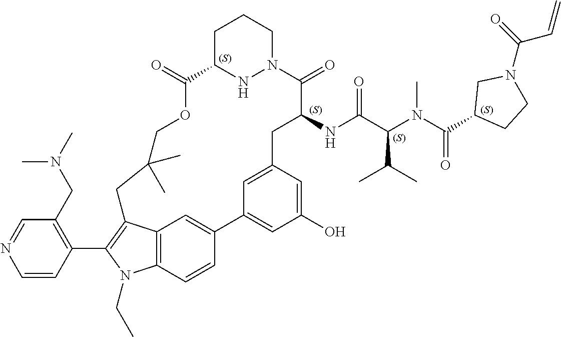 |
| A129 | 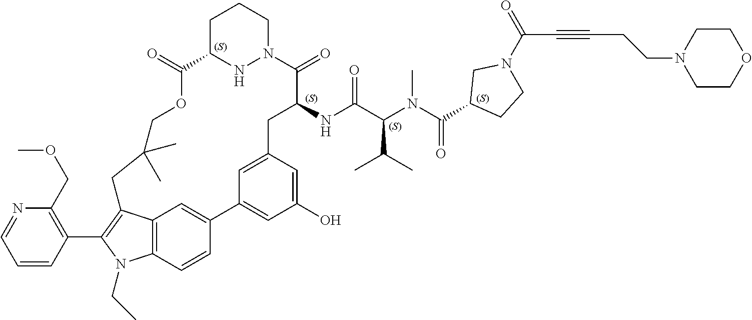 |
| A130 | 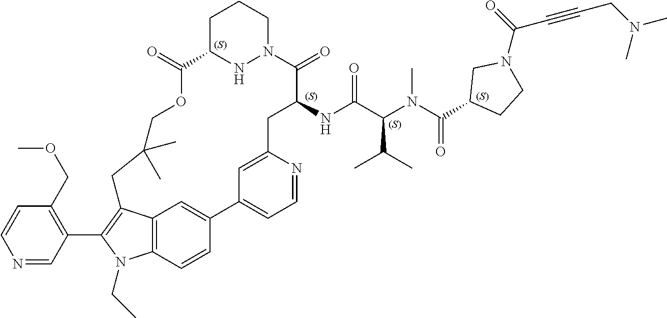 |
| A131 | 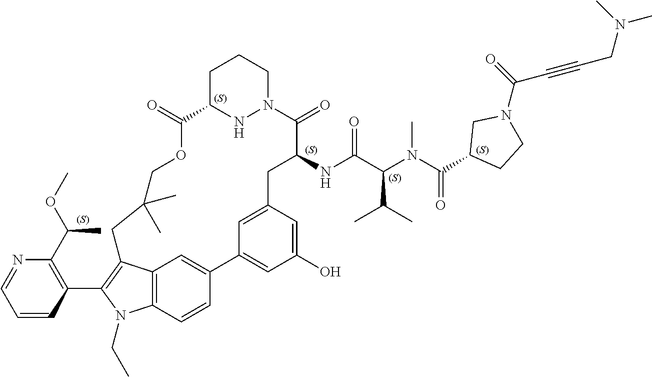 |
| A132 | 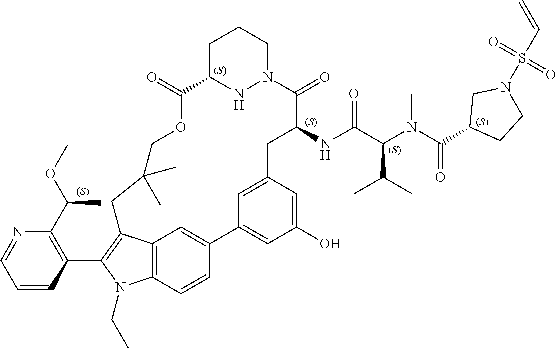 |
| A133 | 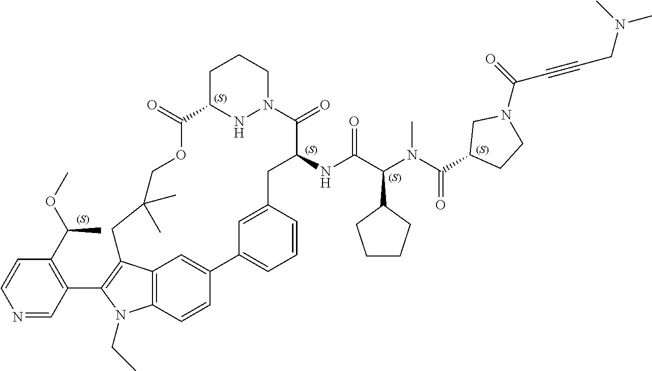 |
| A134 | 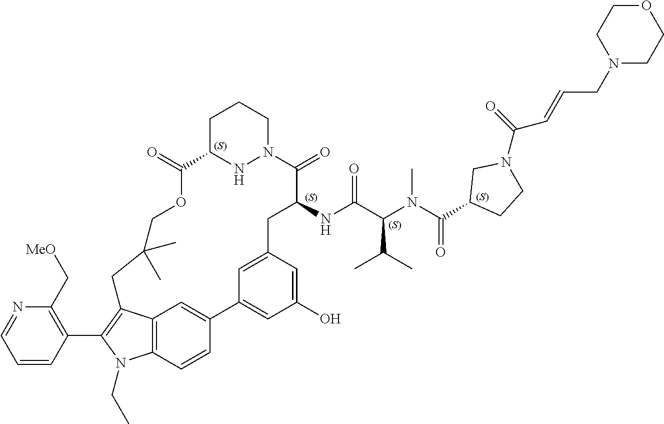 |
| A135 | 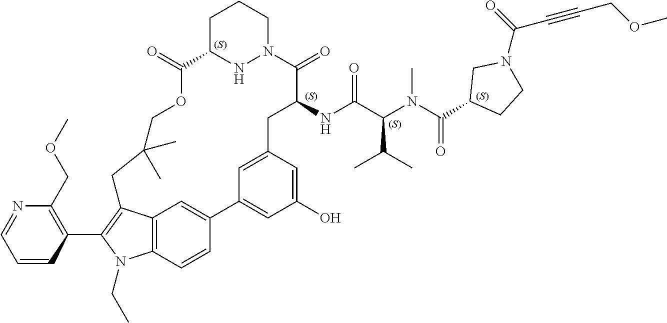 |
| A136 | 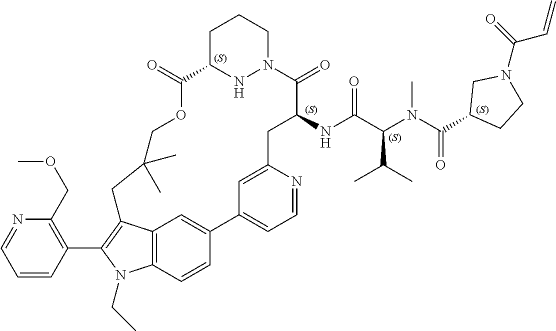 |
| A137 | 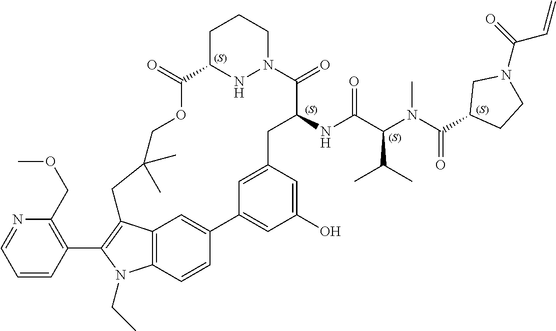 |
| A138 | 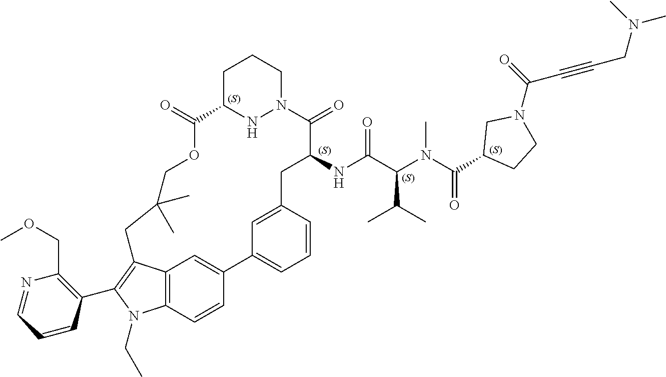 |
| A139 | 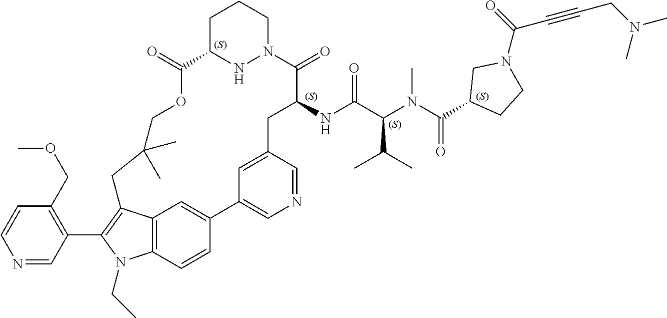 |
| A140 | 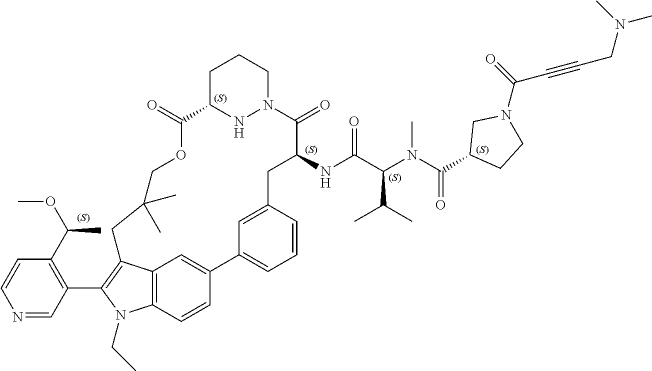 |
| A141 | 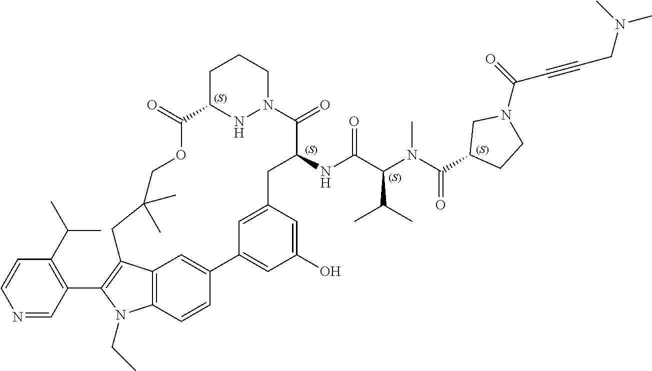 |
| A142 | 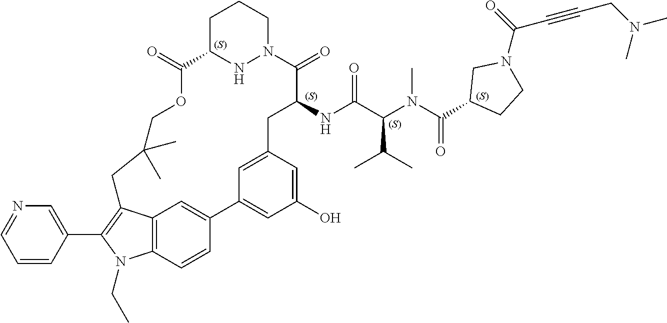 |
| A143 | 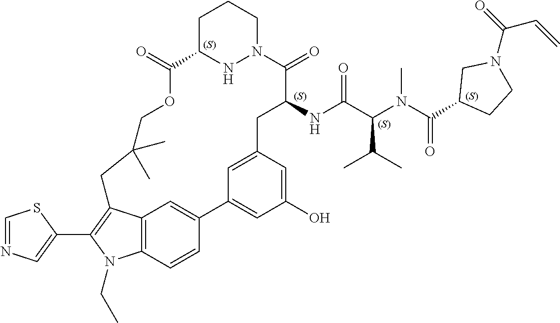 |
| A144 | 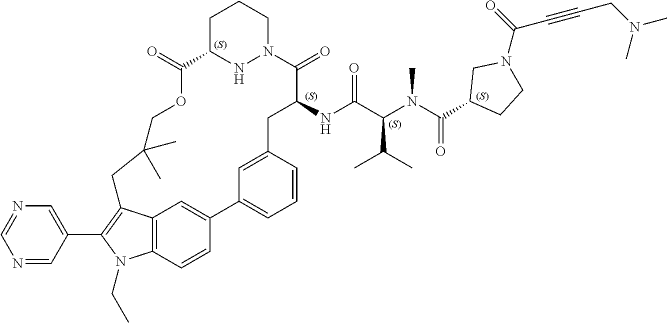 |
| A145 | 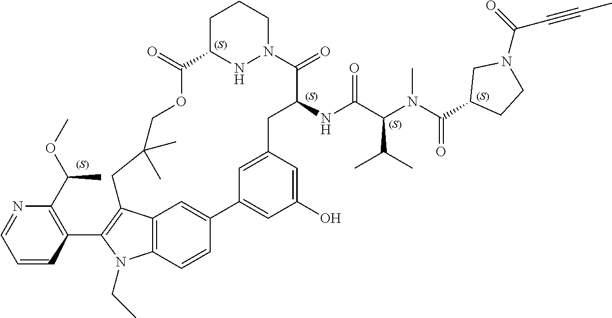 |
| A146 | 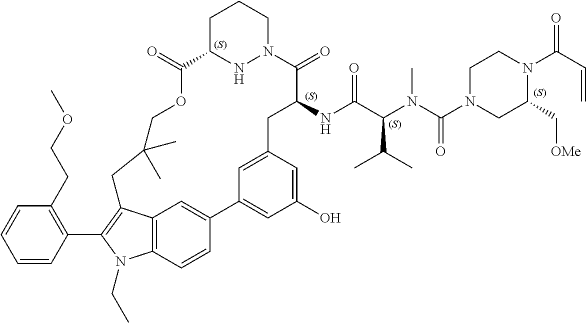 |
| A147 | 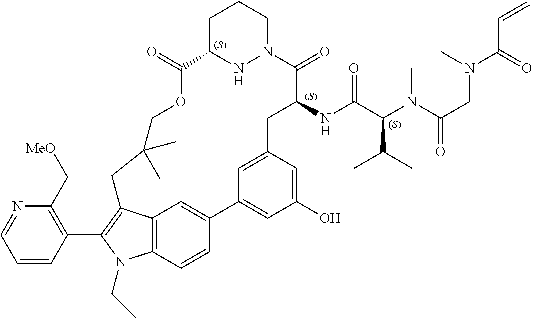 |
| A148 | 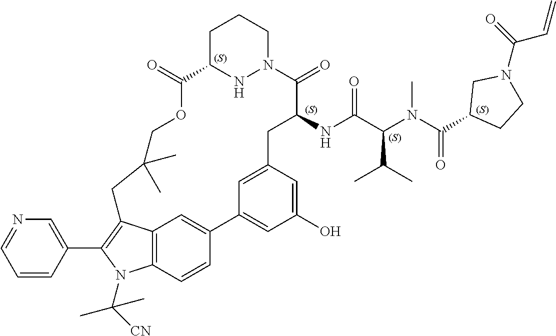 |
| A149 | 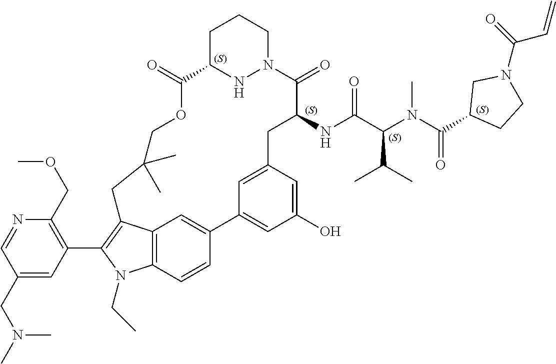 |
| A150 | 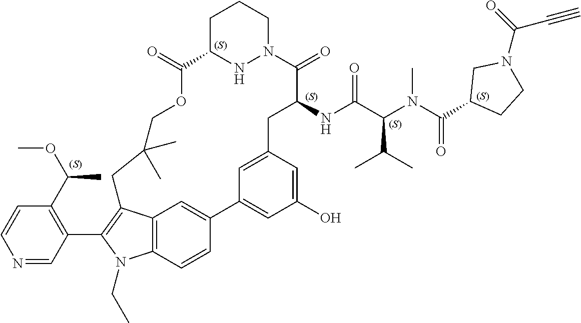 |
| A151 | 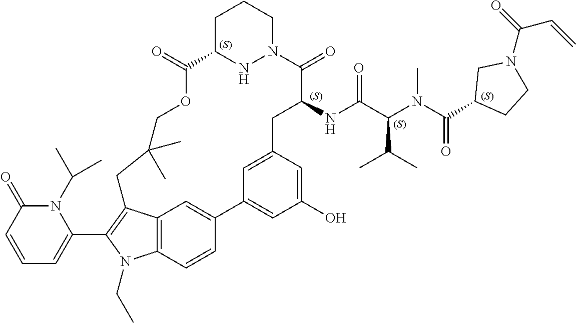 |
| A152 | 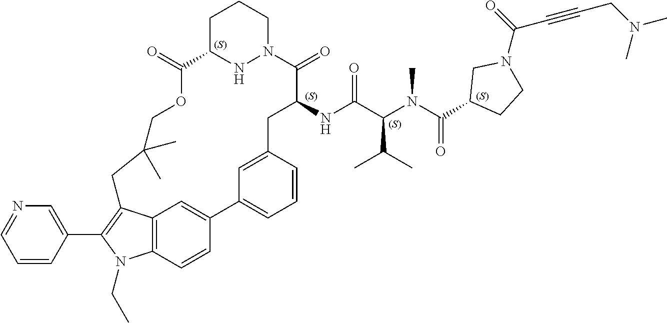 |
| A153 | 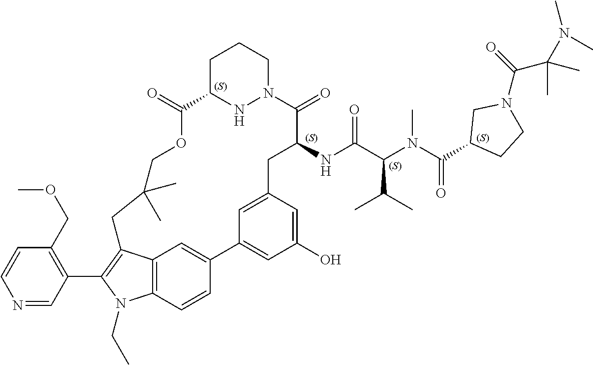 |
| A154 | 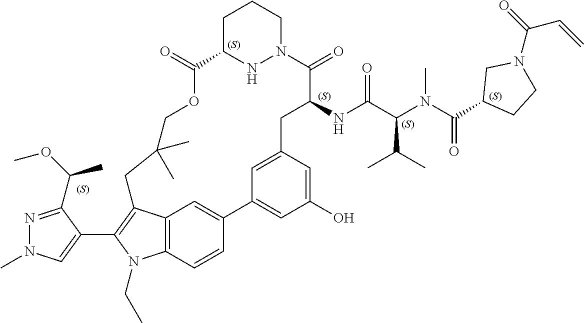 |
| A155 | 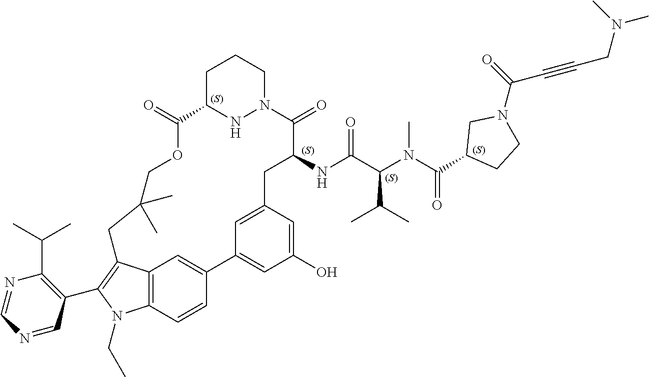 |
| A156 | 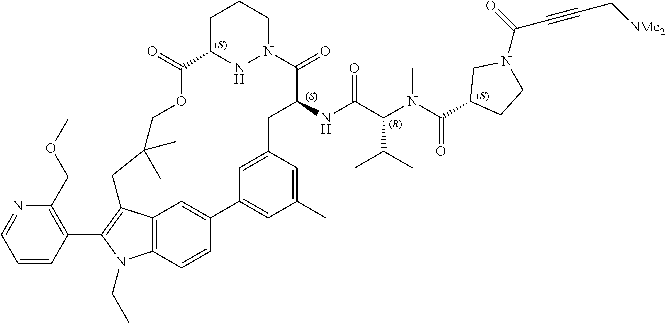 |
| A157 | 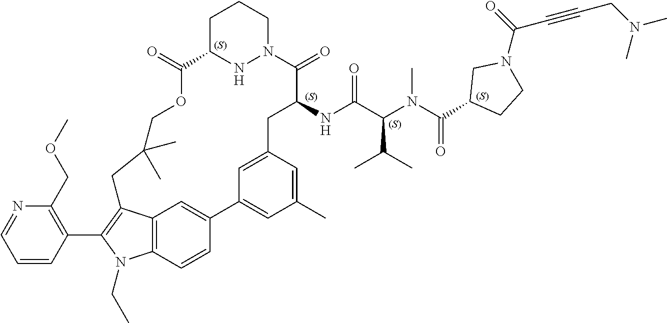 |
| A158 | 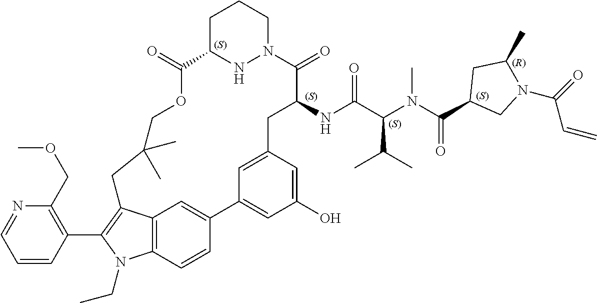 |
| A159 | 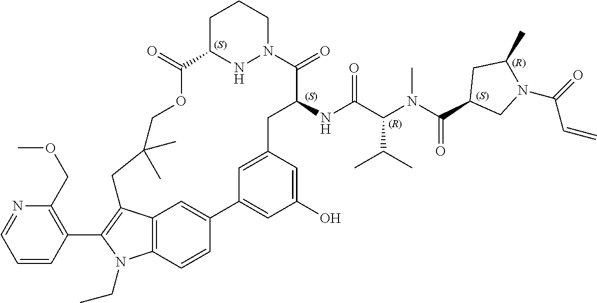 |
| A160 | 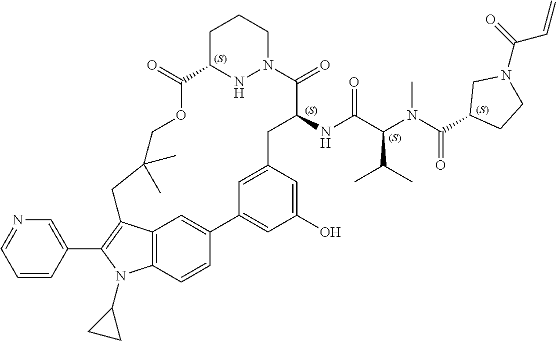 |
| A161 | 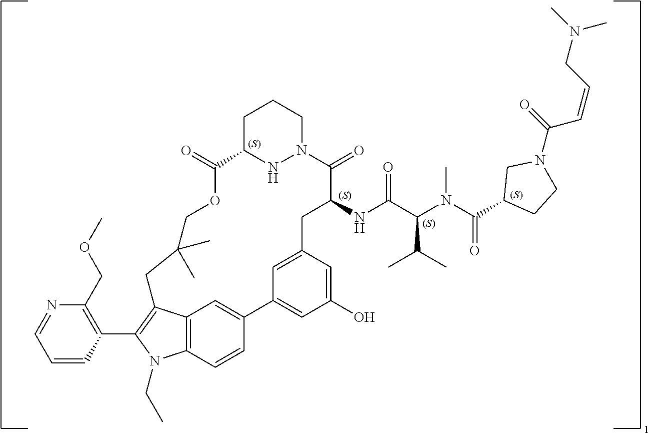 |
| A162 | 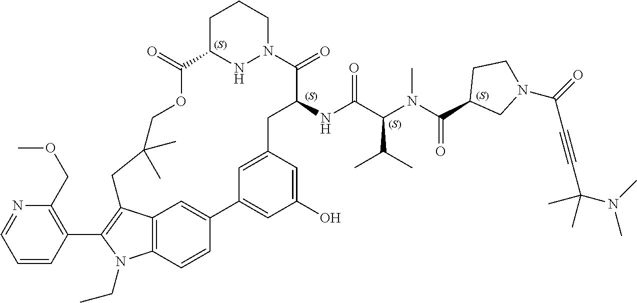 |
| A163 | 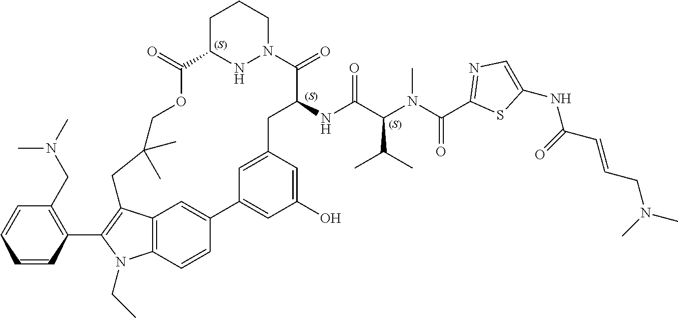 |
| A164 | 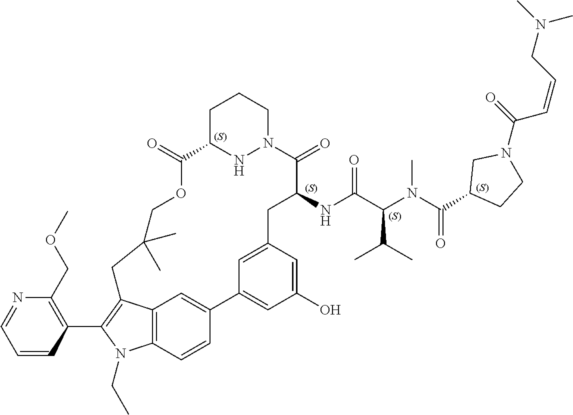 |
| A165 | 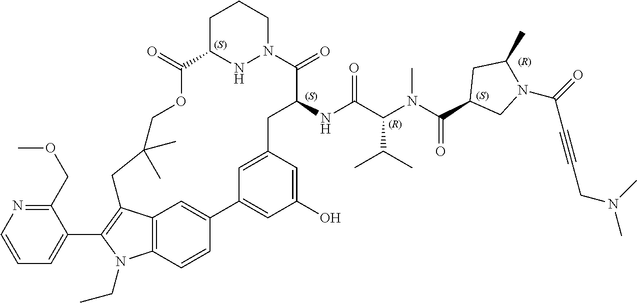 |
| A166 | 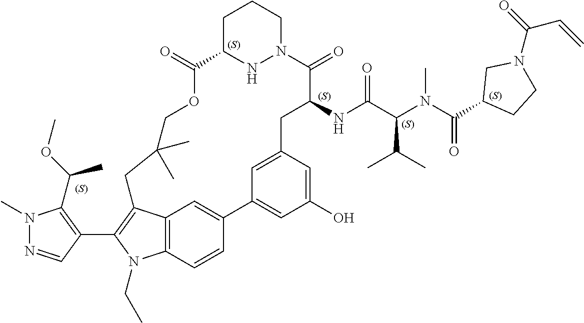 |
| A167 | 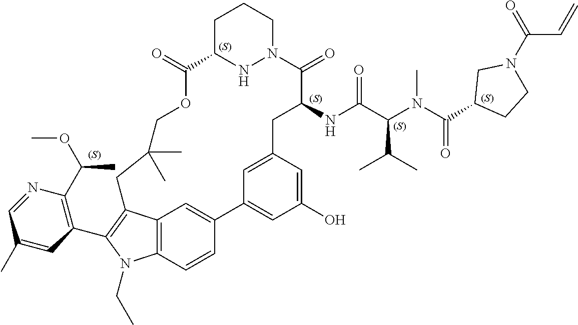 |
| A168 | 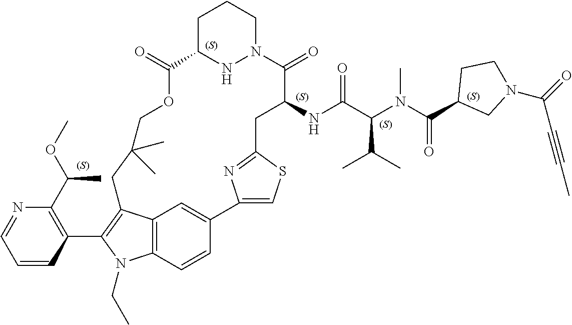 |
| A169 | 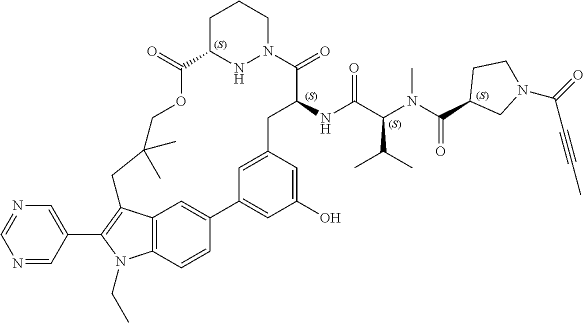 |
| A170 | 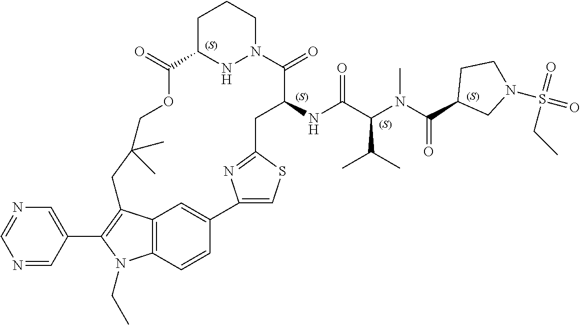 |
| A171 | 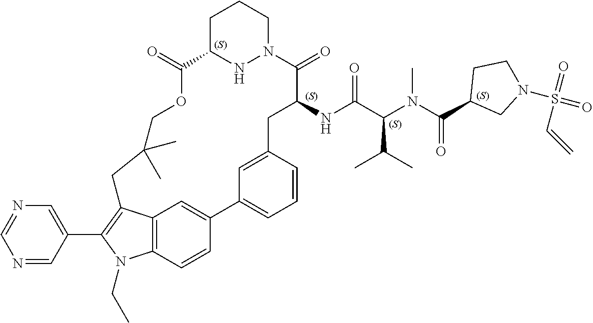 |
| A172 | 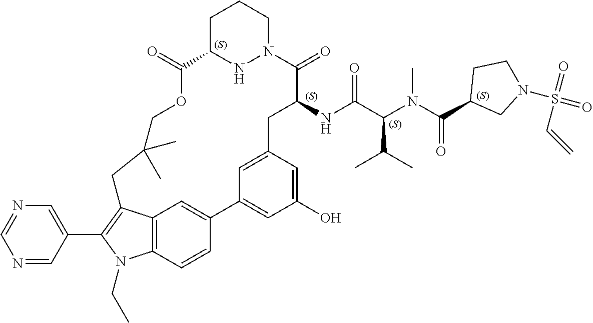 |
| A173 | 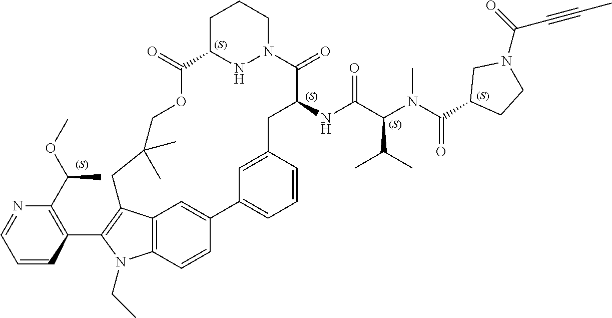 |
| A174 | 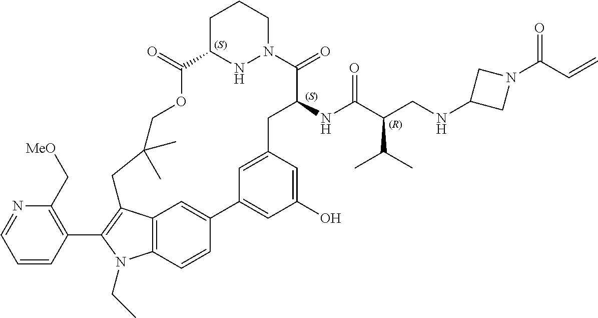 |
| A175 | 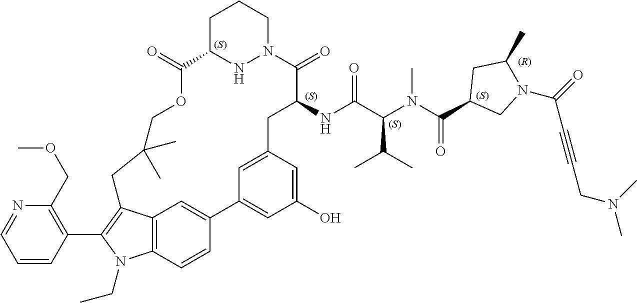 |
| A176 | 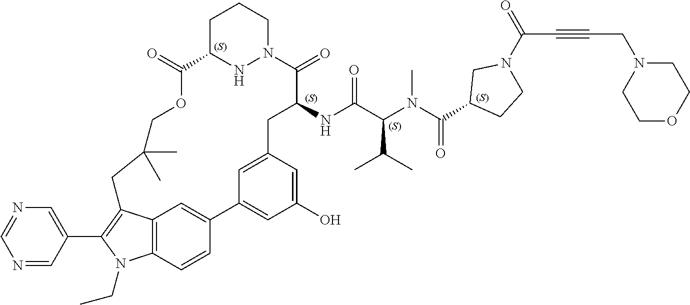 |
| A177 | 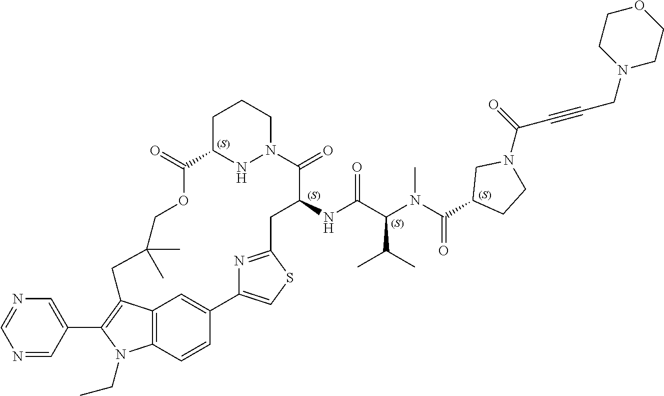 |
| A178 | 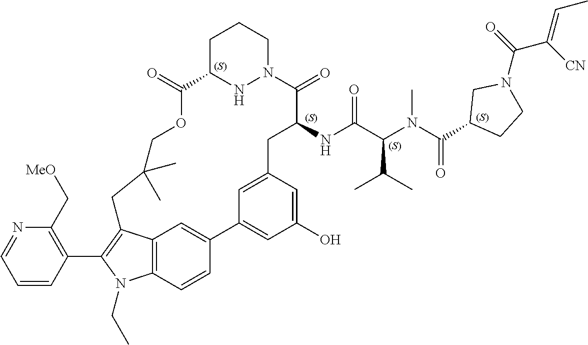 |
| A179 | 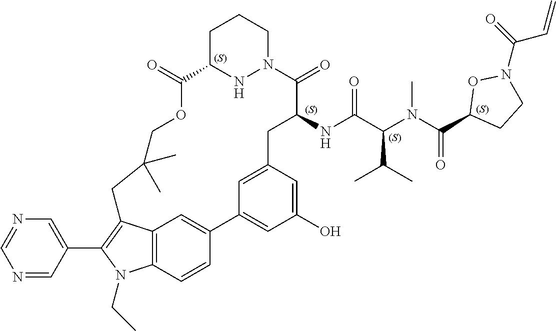 |
| A180 | 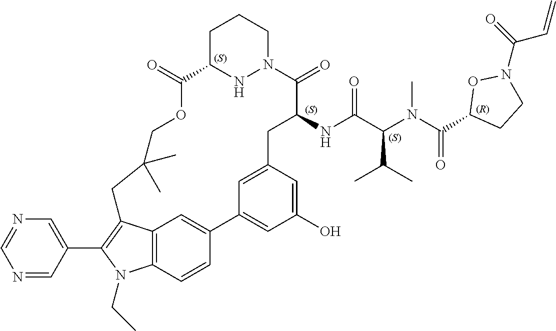 |
| A181 | 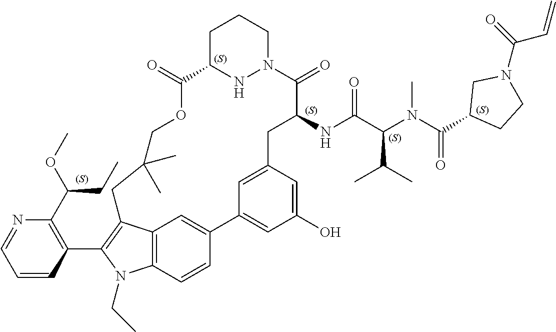 |
| A182 | 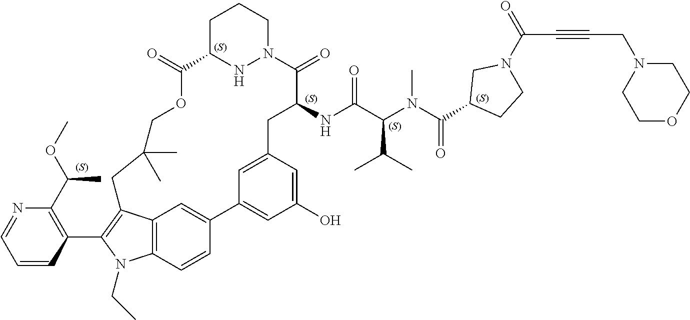 |
| A183 | 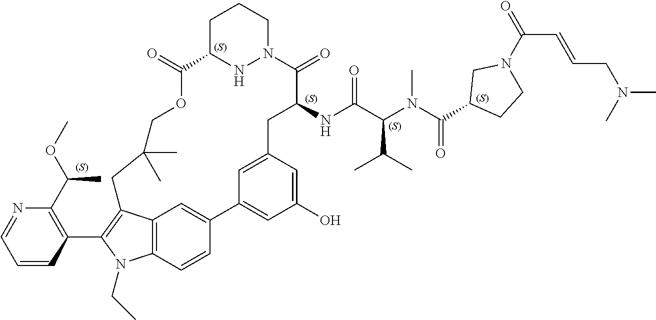 |
| A184 | 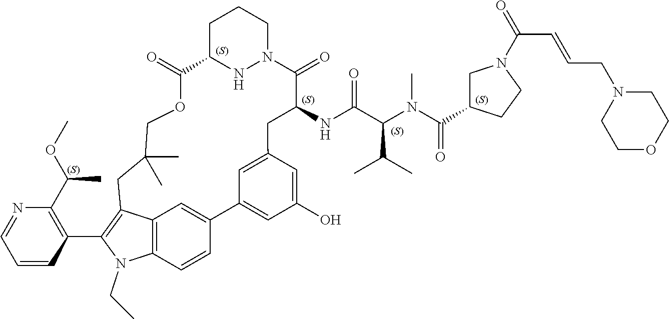 |
| A185 | 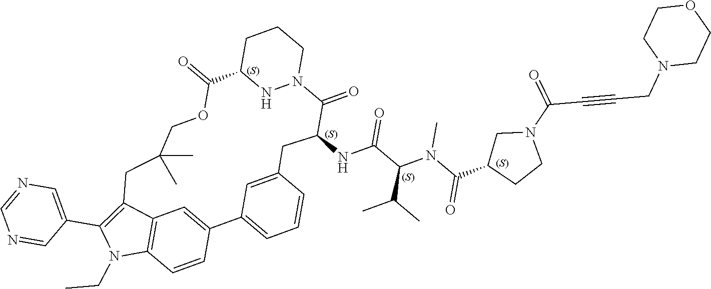 |
| A186 | 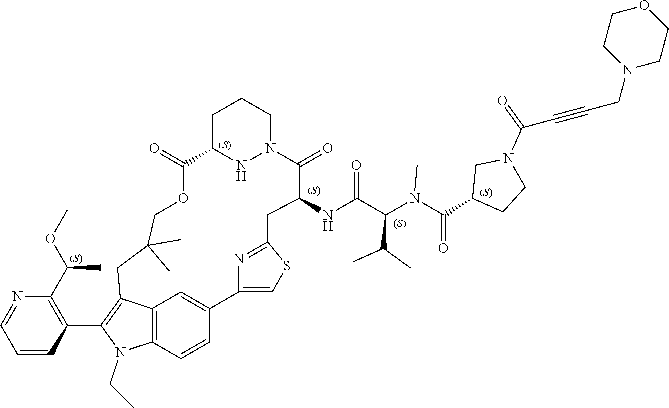 |
| A187 | 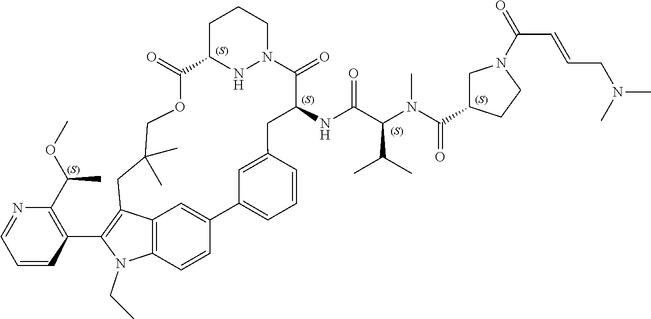 |
| A188 | 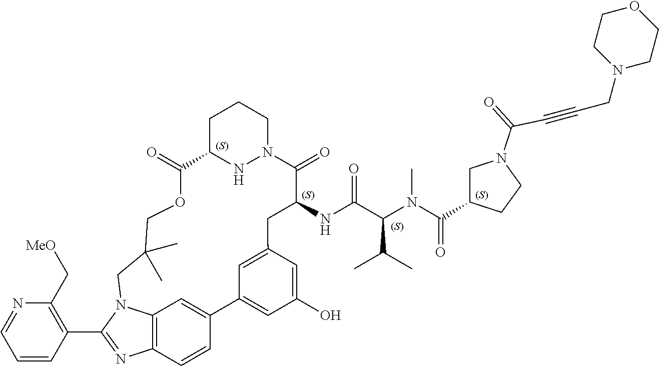 |
| A189 | 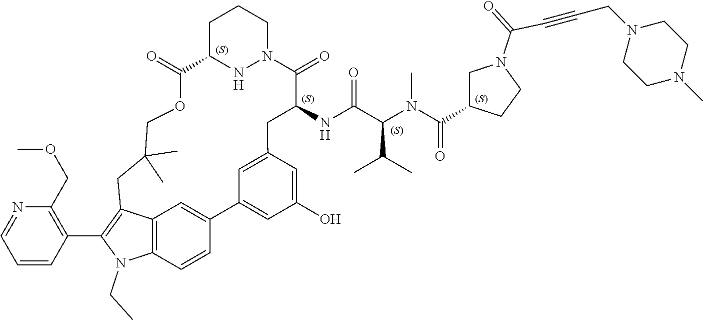 |
| A190 | 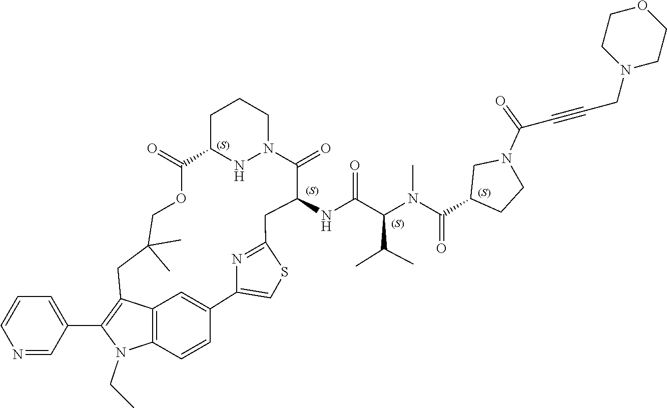 |
| A191 | 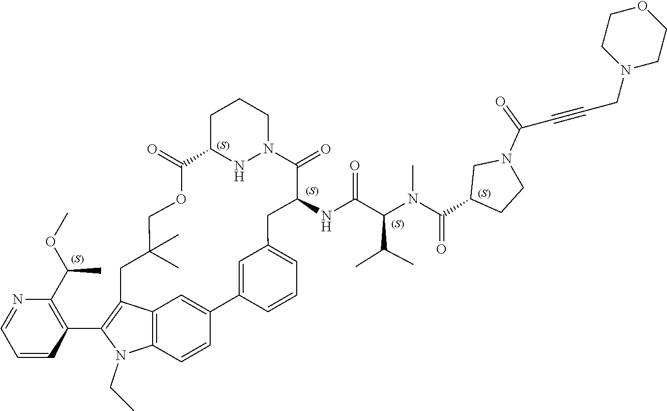 |
| A192 | 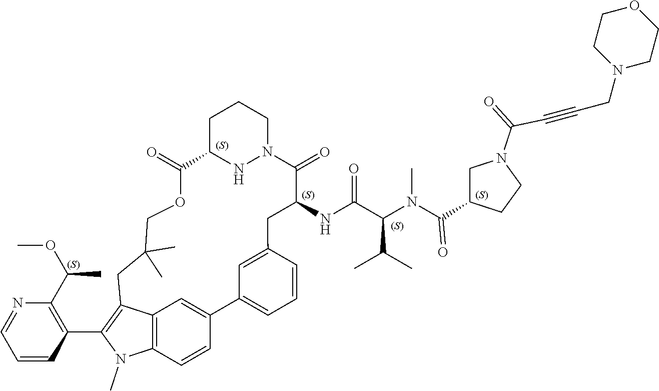 |
| A193 | 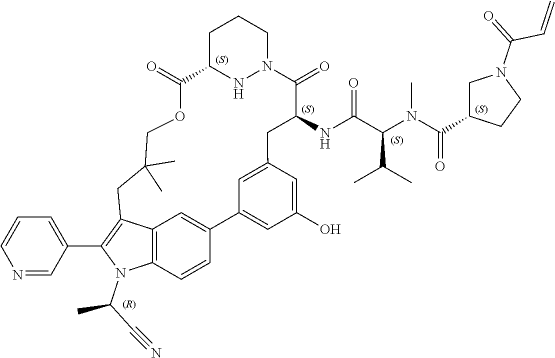 |
| A194 | 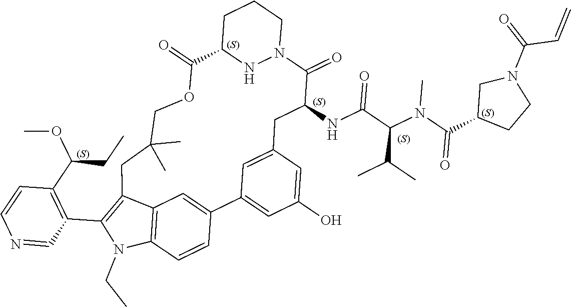 |
| A195 | 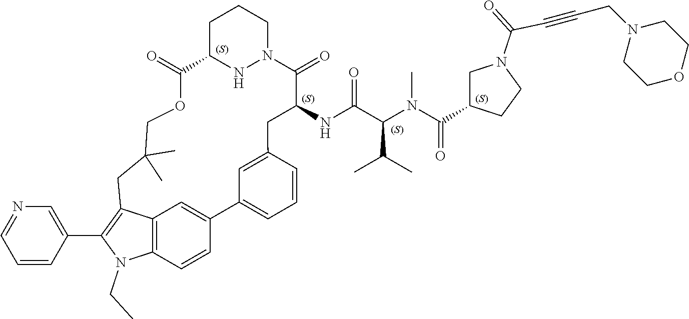 |
| A196 | 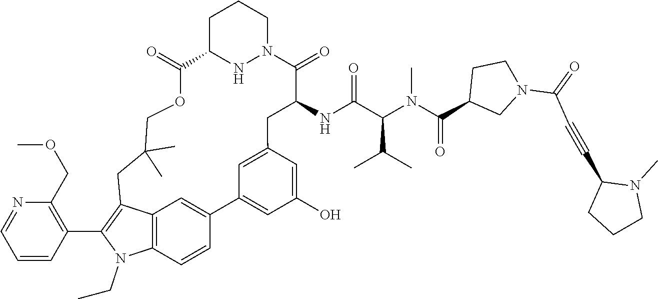 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| A197 | 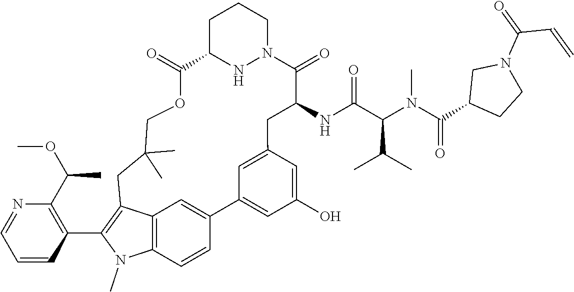 |
| A198 | 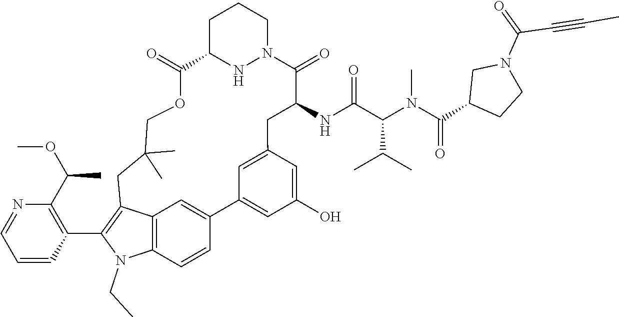 |
| A199 | 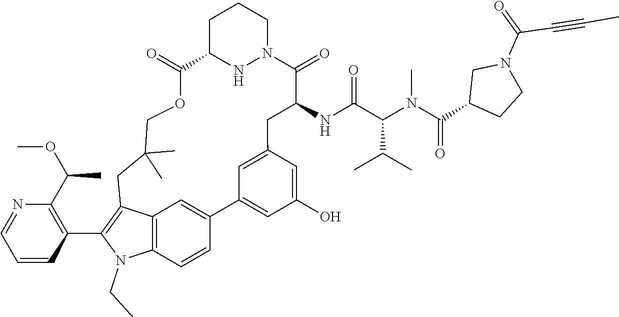 |
| A200 | 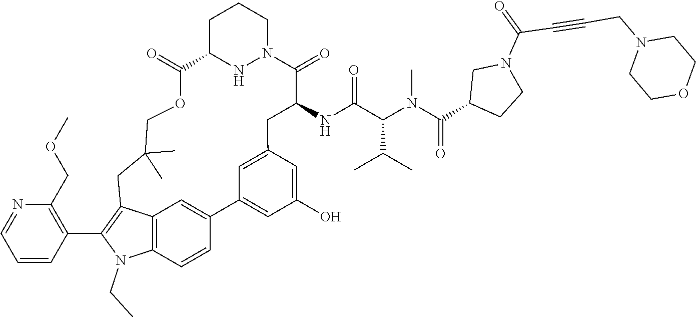 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN3CCOCC3)C1)C(=O)N1CCC[C@H](N1)C(=O)OCC(C)(C)C2] |
| A201 | 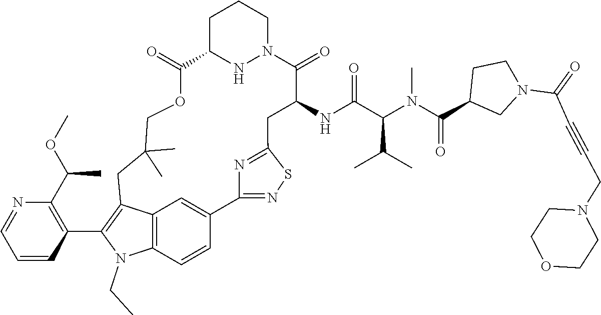 |
| A202 | 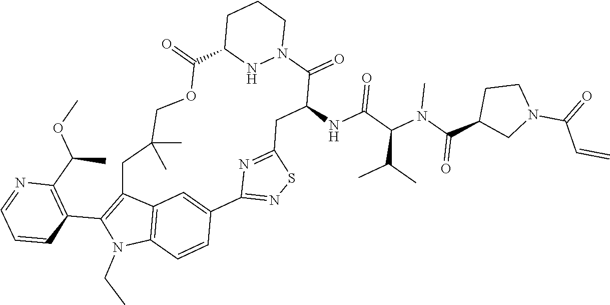 |
| A203 | 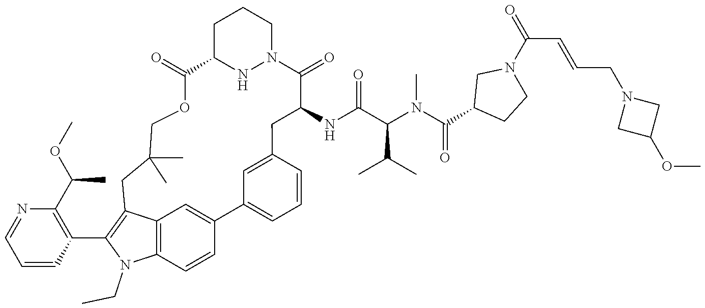 |
| A204 | 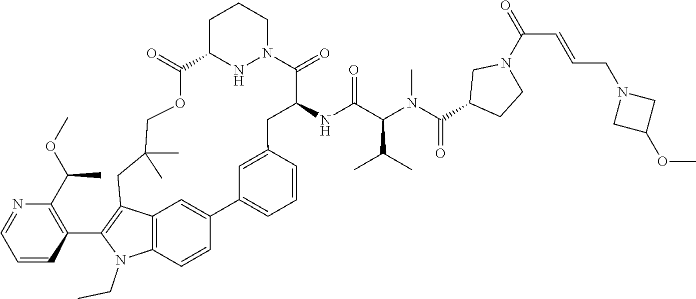 |
| A205 | 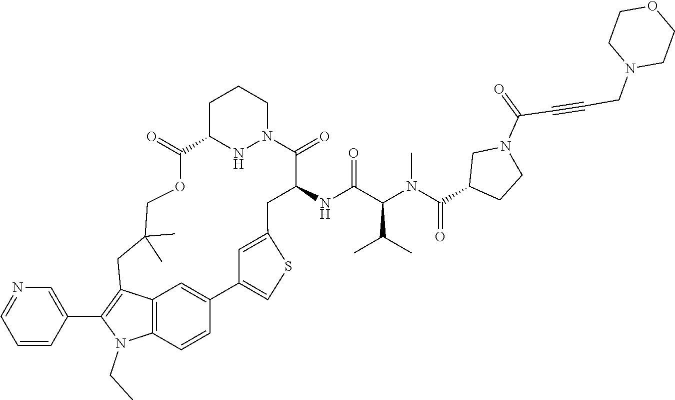 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A206 | 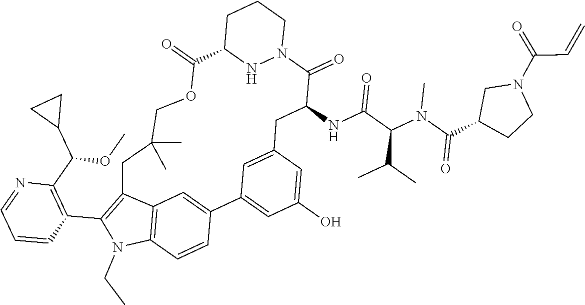 |
| A207 | 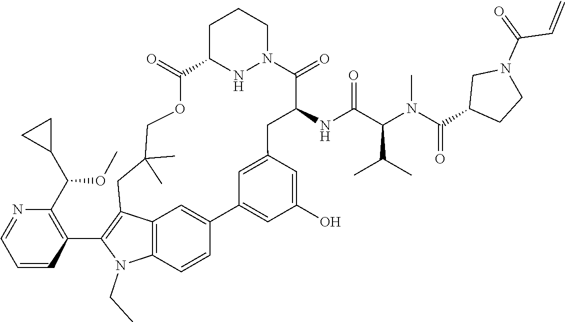 |
| A208 | 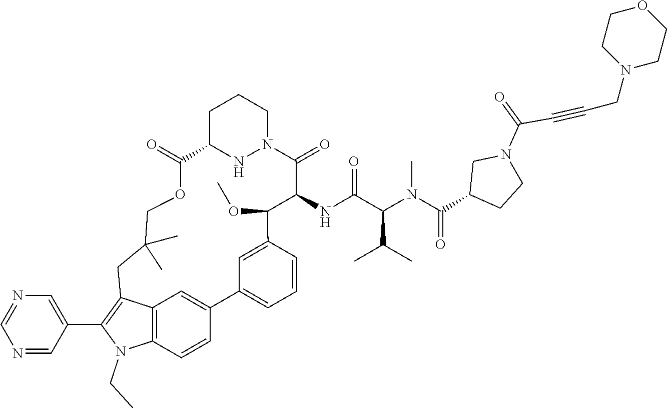 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| A209 | 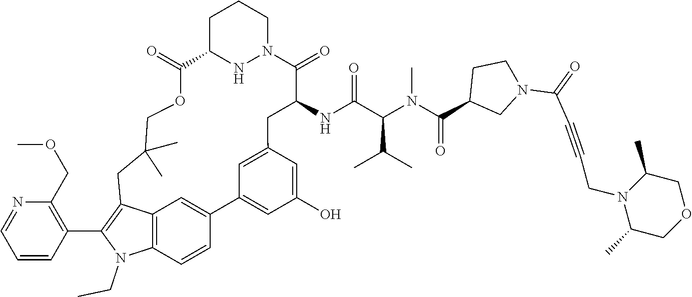 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| A210 | 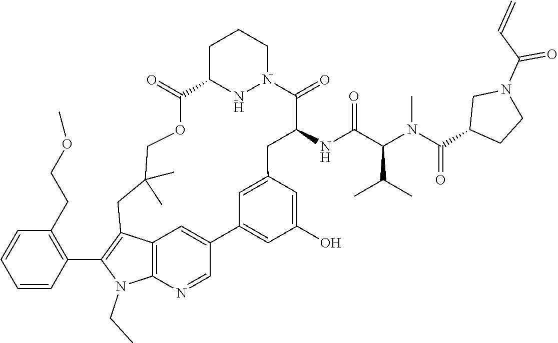 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| A211 | 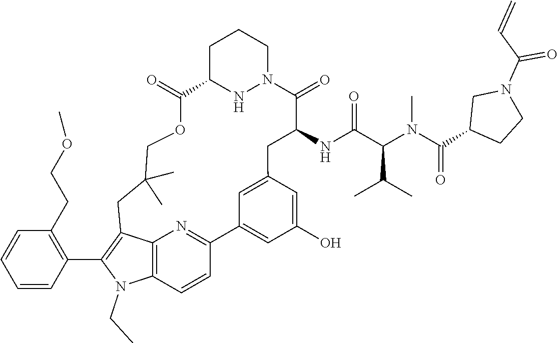 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| A212 | 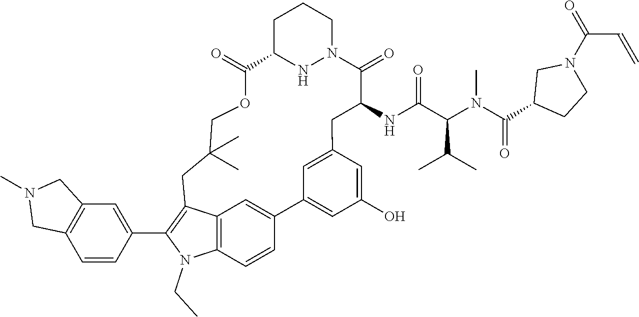 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| A213 | 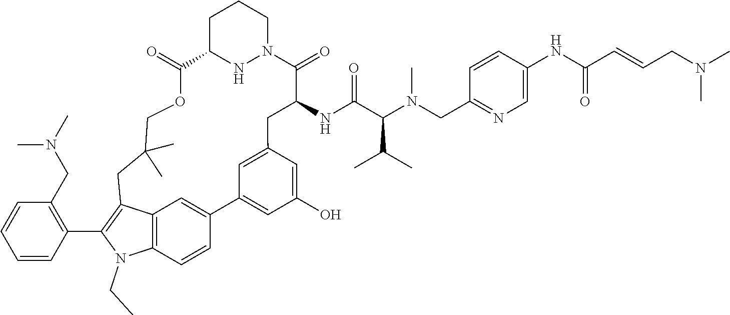 [SMILES: CCn1c(-c2ccccc2CN(C)C)c2c3cc(ccc31)-c1cc(O)cc(c1)C[C@H](NC(=O)[C@H](C(C)C)N(C)Cc1ccc(NC(=O)/C=C/CN(C)C)cn1)C(=O)N1CCC[C@H](N1)C(=O)OCC(C)(C)C2] |
| A214 | 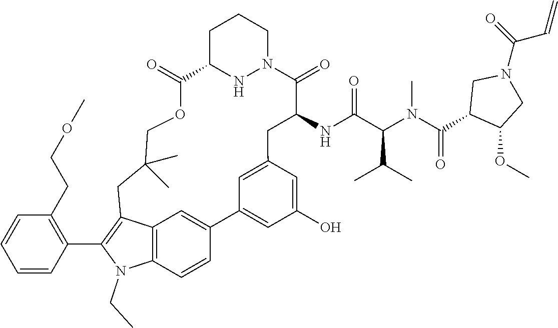 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| A215 | 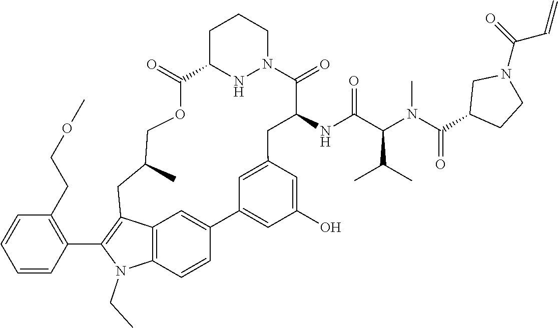 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| A216 | 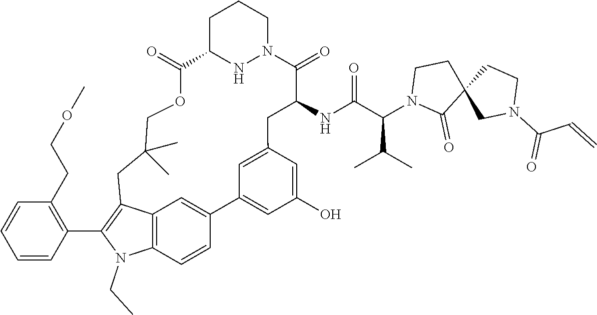 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| A217 | 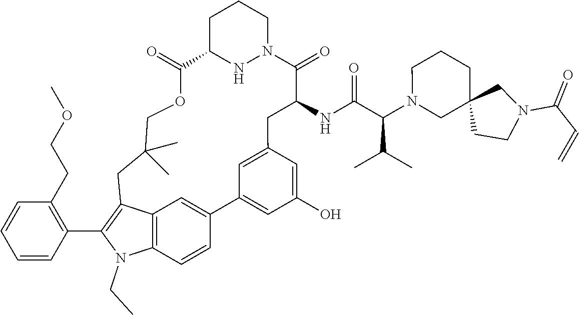 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2)C1] |
| A218 | 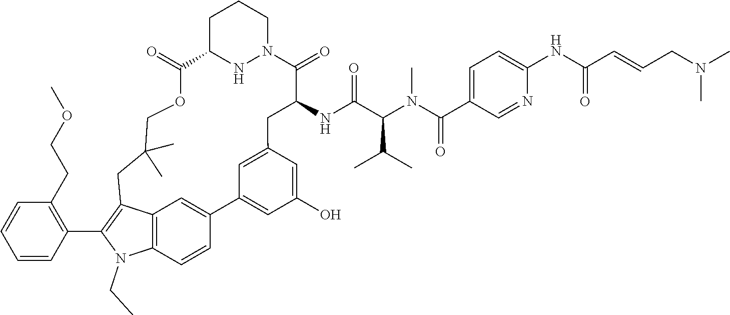 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| A219 | 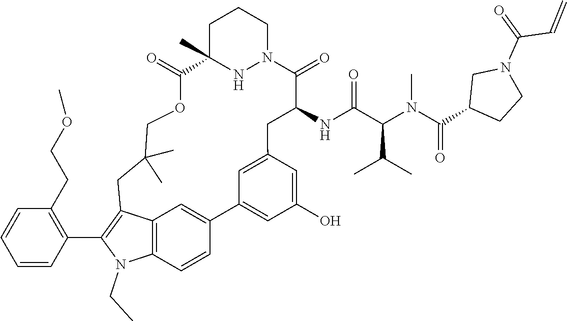 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| A220 | 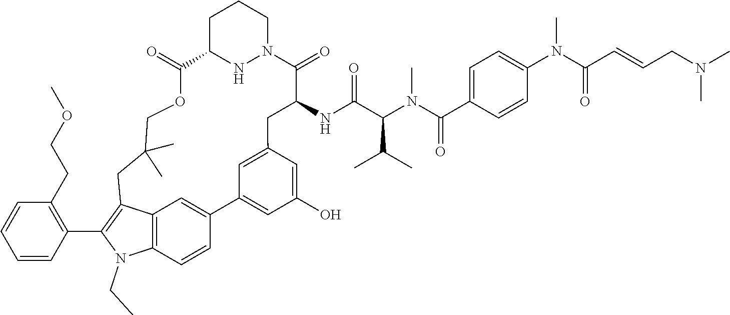 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| A221 | 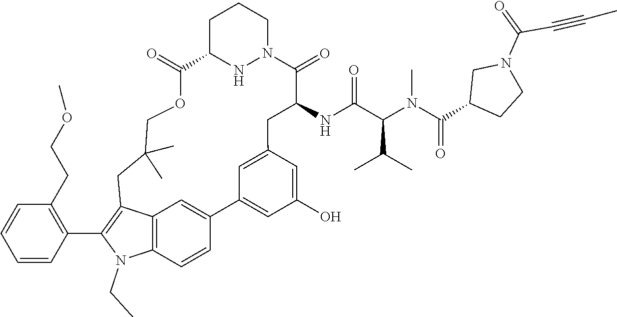 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| A222 | 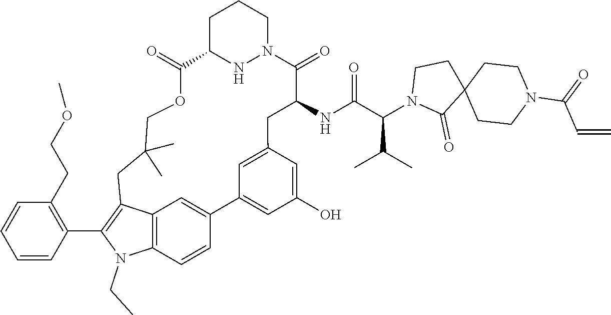 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| A223 | 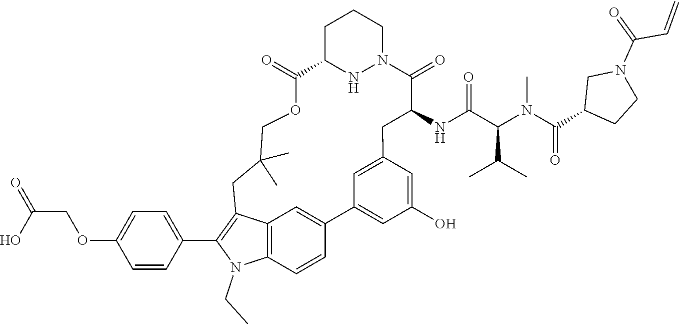 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| A224 | 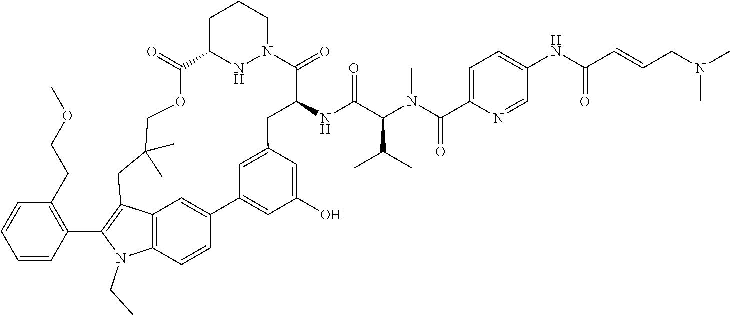 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| A225 | 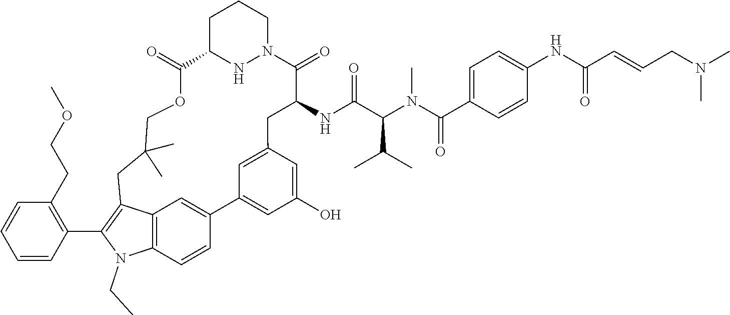 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| A226 | 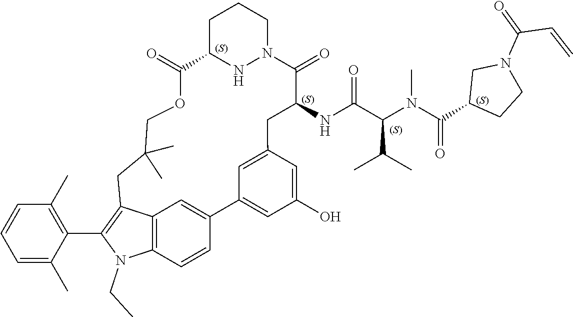 |
| A227 | 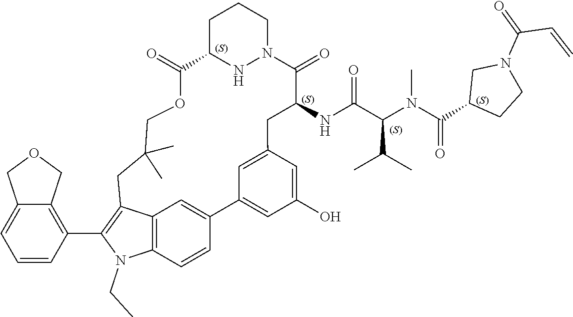 |
| A228 | 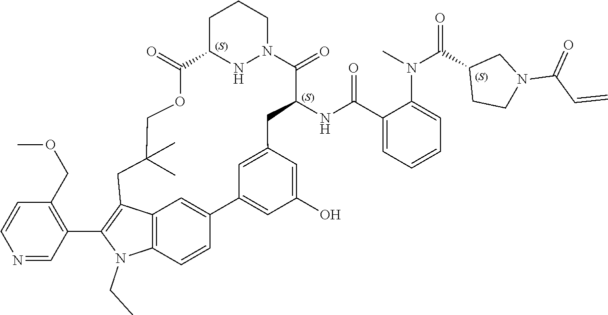 |
| A229 | 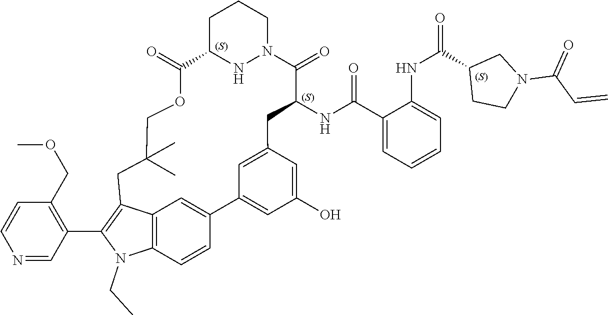 |
| A230 | 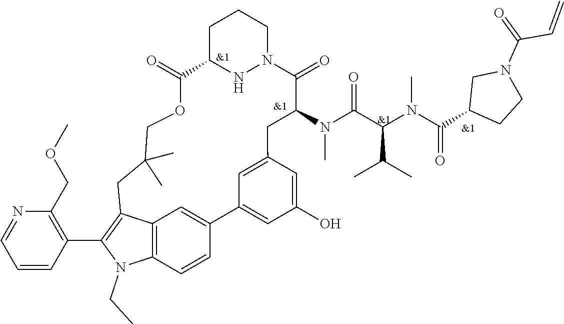 [SMILES: C=CC(=O)N1CC[C@H](C(=O)N(C)[C@H](C(=O)N(C)[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A231 | 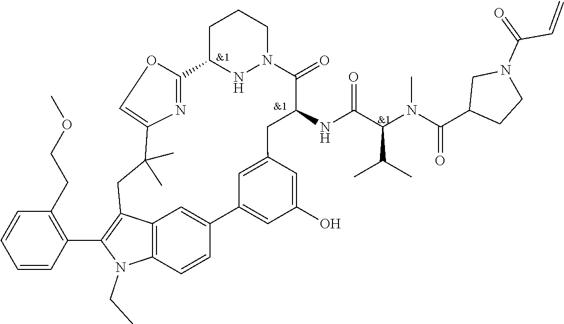 [SMILES: C=CC(=O)N1CCC(C(=O)N(C)[C@H](C(=O)N[C@H]2Cc3cc(O)cc(c3)-c3ccc4c(c3)c(c(-c3ccccc3CCOC)n4CC)CC(C)(C)c3coc(n3)[C@@H]3CCCN(N3)C2=O)C(C)C)C1] |
| A232 | 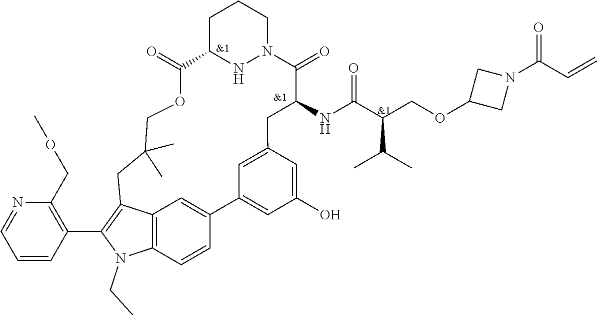 [SMILES: C=CC(=O)N1CC(OC[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A233 | 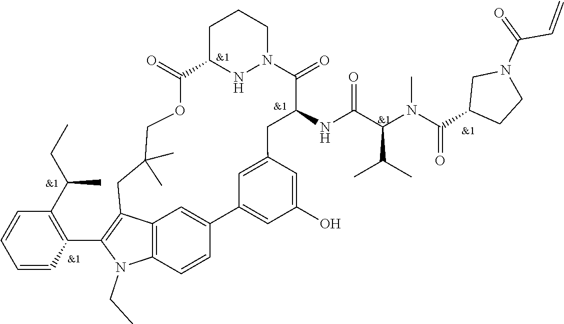 |
| A234 | 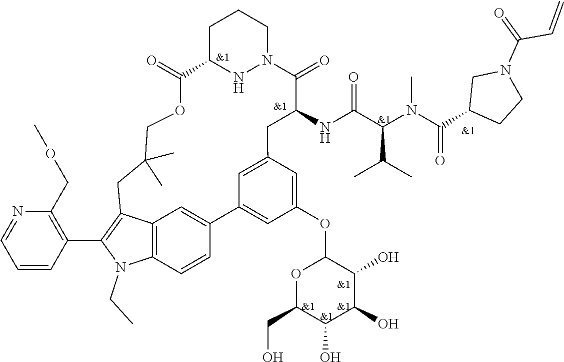 [SMILES: C=CC(=O)N1CC[C@H](C(=O)N(C)[C@H](C(=O)N[C@H]2Cc3cc(OC4O[C@H](CO)[C@@H](O)[C@H](O)[C@H]4O)cc(c3)-c3ccc4c(c3)c(c(-c3cccnc3COC)n4CC)CC(C)(C)COC(=O)[C@@H]3CCCN(N3)C2=O)C(C)C)C1] |
| A235 | 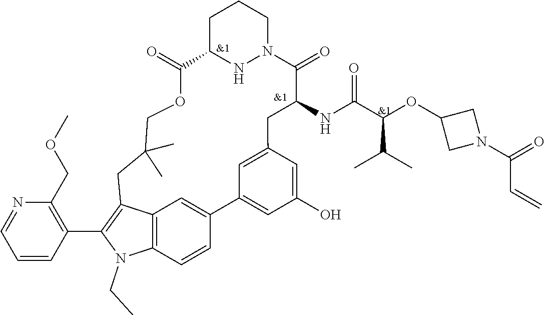 [SMILES: C=CC(=O)N1CC(O[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| A236 | 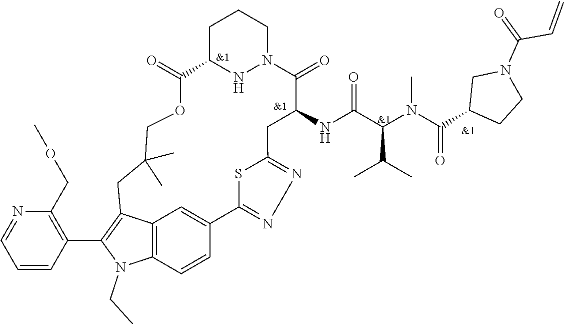 [SMILES: C=CC(=O)N1CC[C@H](C(=O)N(C)[C@H](C(=O)N[C@H]2Cc3nnc(s3)-c3ccc4c(c3)c(c(-c3cccnc3COC)n4CC)CC(C)(C)COC(=O)[C@@H]3CCCN(N3)C2=O)C(C)C)C1] |
| A237 | 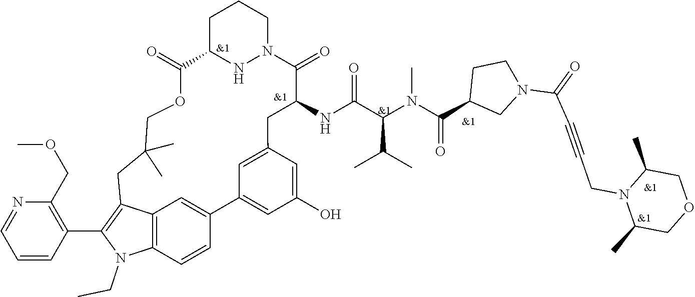 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@H](C)COC[C@@H]3C)C1)C(=O)N1CCC[C@H](N1)C(=O)OCC(C)(C)C2] |
| A238 | 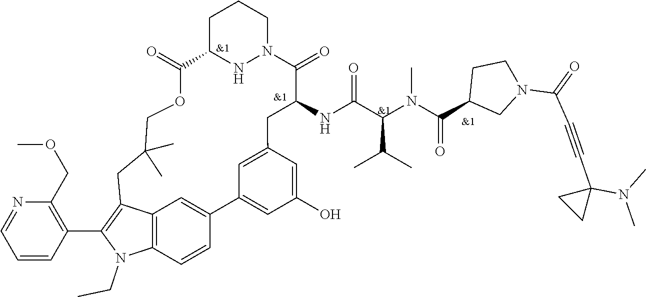 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)CC3)C1)C(=O)N1CCC[C@H](N1)C(=O)OCC(C)(C)C2] |
| A239 | 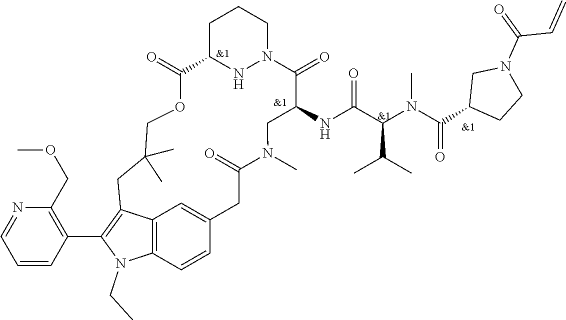 [SMILES: C=CC(=O)N1CC[C@H](C(=O)N(C)[C@H](C(=O)N[C@H]2CN(C)C(=O)Cc3ccc4c(c3)c(c(-c3cccnc3COC)n4CC)CC(C)(C)COC(=O)[C@@H]3CCCN(N3)C2=O)C(C)C)C1] |
| A240 | 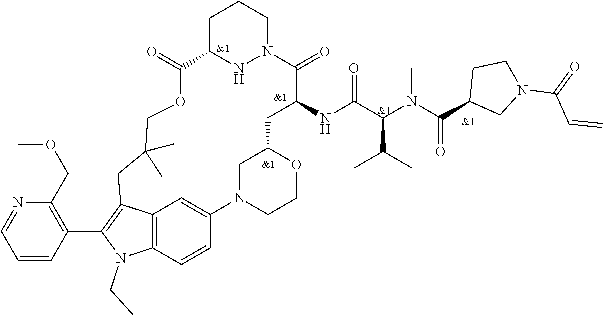 [SMILES: C=CC(=O)N1CC[C@H](C(=O)N(C)[C@H](C(=O)N[C@H]2C[C@H]3CN(CCO3)c3ccc4c(c3)c(c(-c3cccnc3COC)n4CC)CC(C)(C)COC(=O)[C@@H]3CCCN(N3)C2=O)C(C)C)C1] |
| A241 | 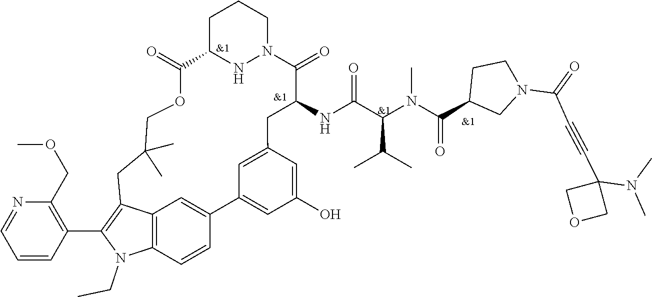 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)COC3)C1)C(=O)N1CCC[C@H](N1)C(=O)OCC(C)(C)C2] |
| A242 | 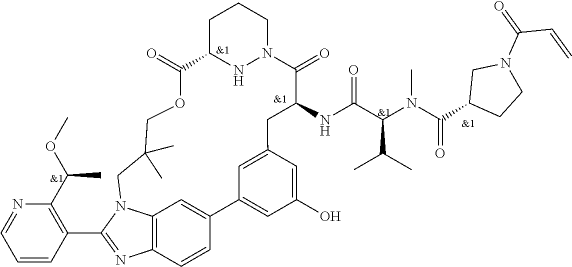 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)n(c4c3)CC(C)(C)COC(=O)[C@@H]3CCCN(N3)C2=O)C(C)C)C1] |
| A243 | 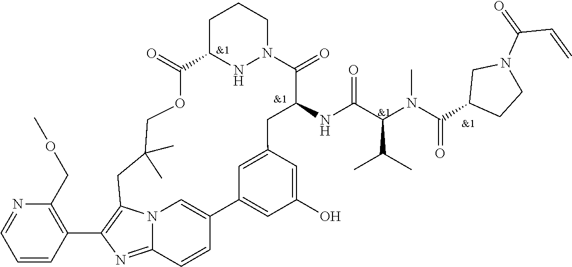 [SMILES: C=CC(=O)N1CC[C@H](C(=O)N(C)[C@H](C(=O)N[C@H]2Cc3cc(O)cc(c3)-c3ccc4nc(-c5cccnc5COC)c(n4c3)CC(C)(C)COC(=O)[C@@H]3CCCN(N3)C2=O)C(C)C)C1] |
| A244 | 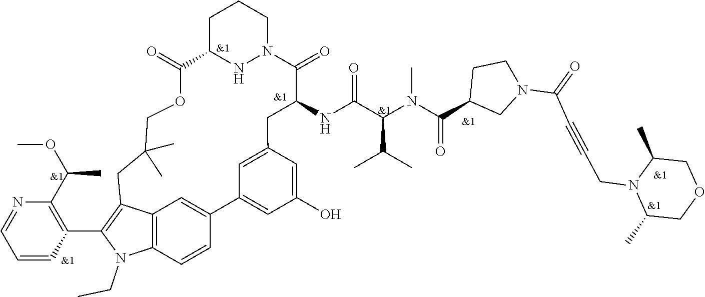 |
| A245 | 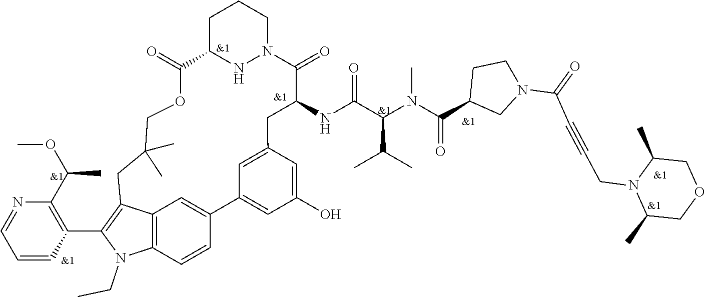 |
| A246 | 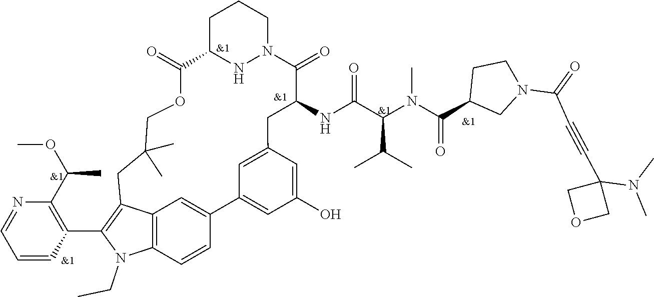 |
| A247 | 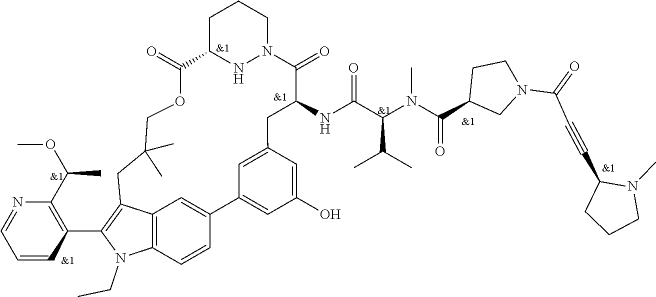 |
| A248 | 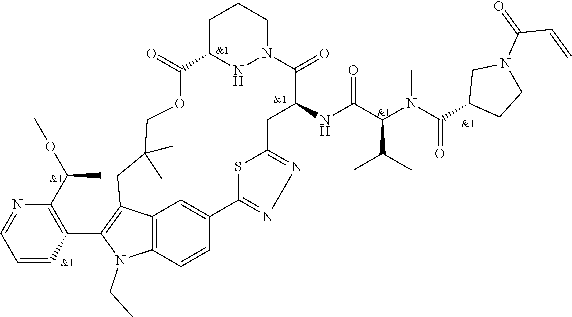 |
| A249 | 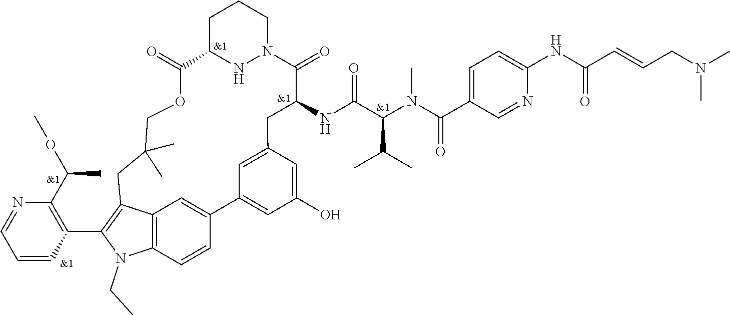 |
| A250 | 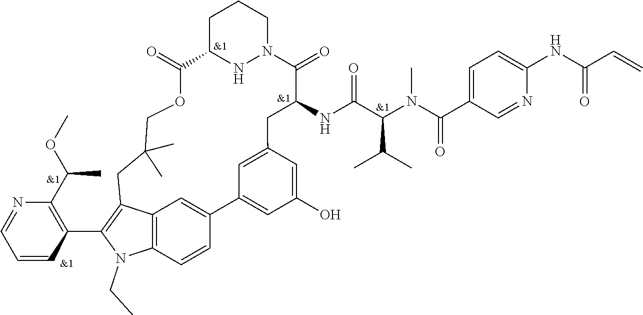 |
| A251 | 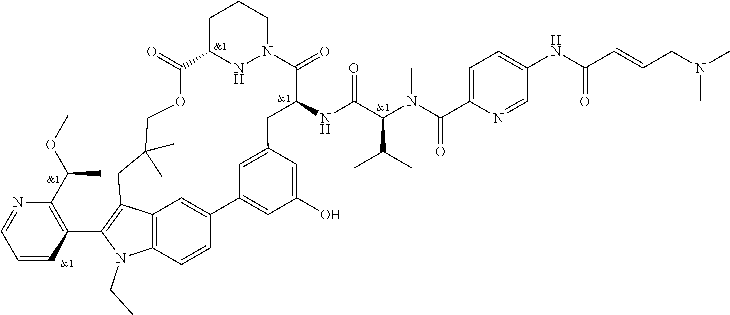 |
| A252 | 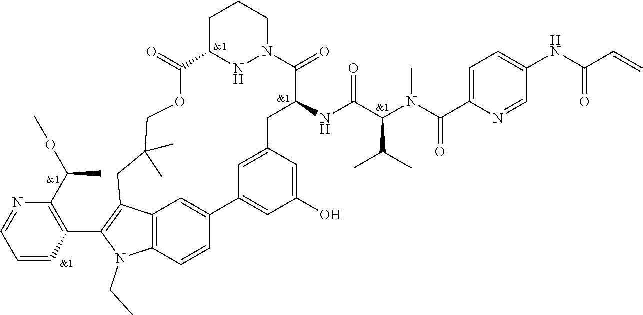 |
| A253 | 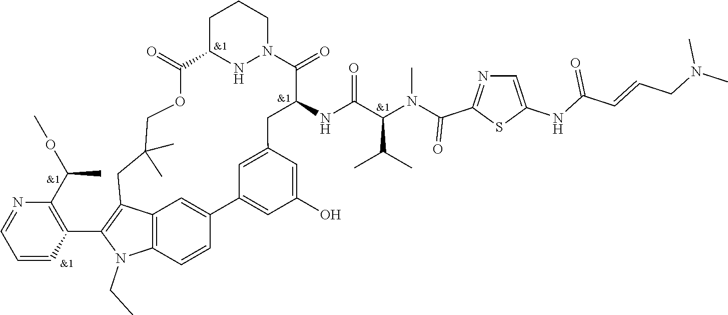 |
| A254 | 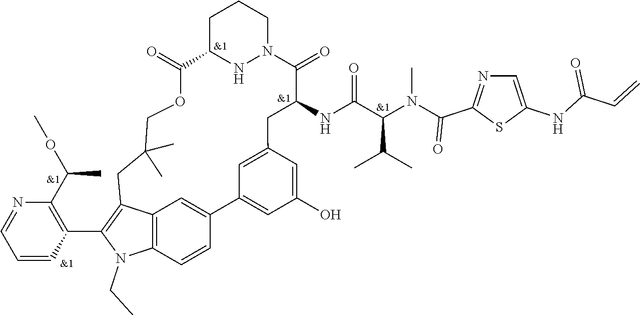 |
| A255 | 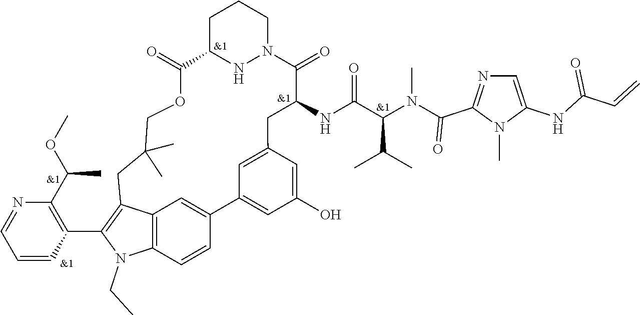 |
| A256 | 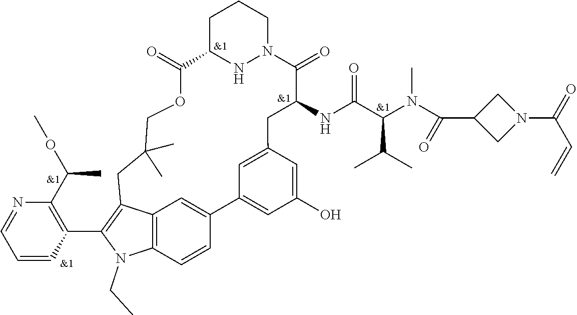 |
| A257 | 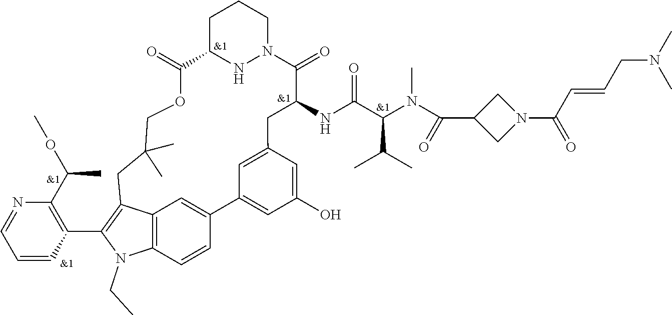 |
| A258 | 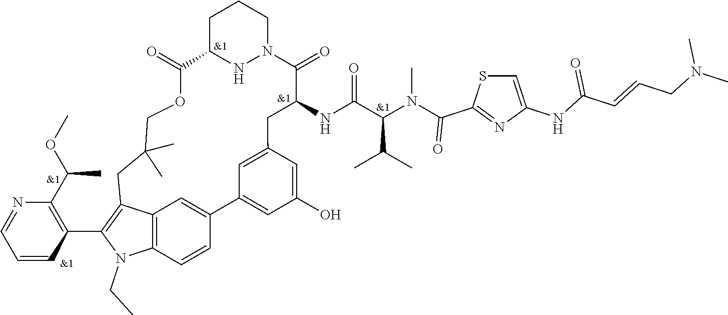 |
| A259 | 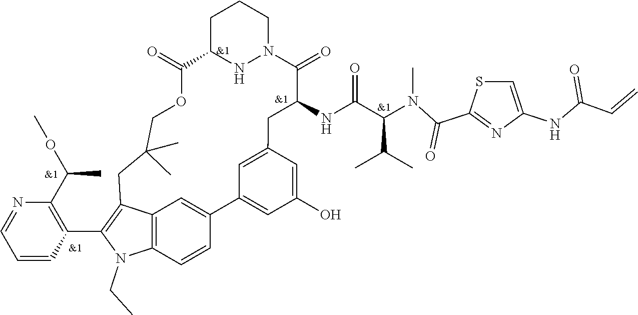 |
| A260 | 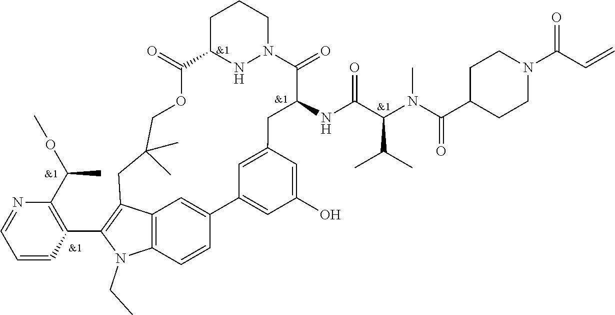 |
| A261 | 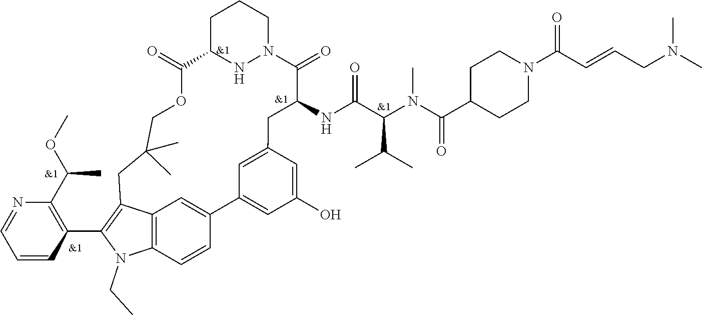 |
| A262 | 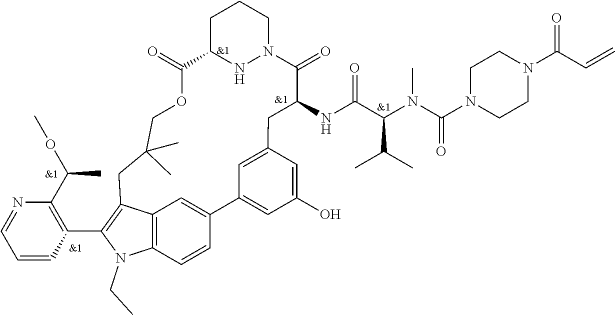 |
| A263 | 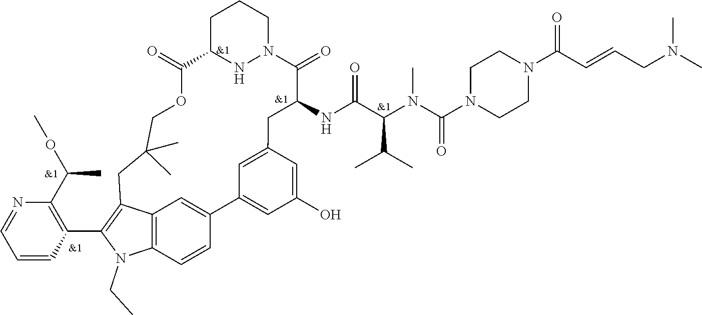 |
| A264 | 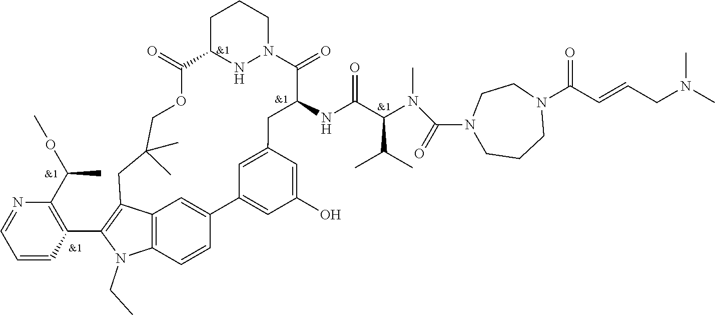 |
| A265 | 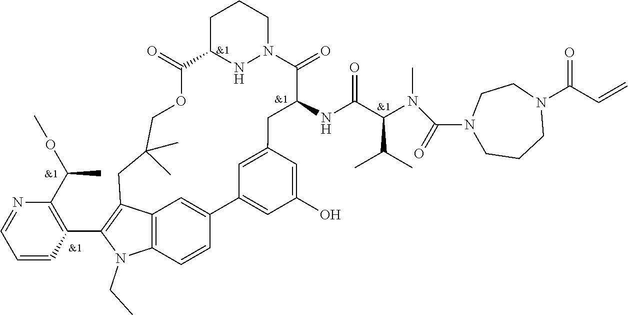 |
| A266 | 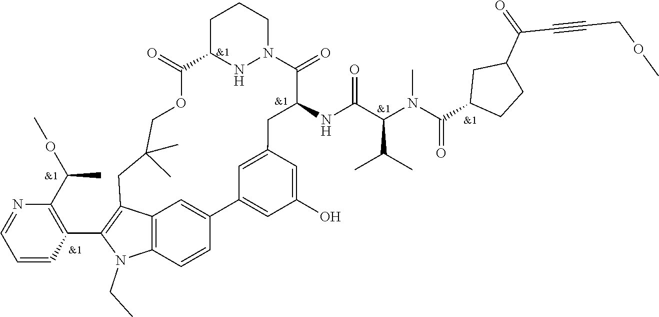 |
| A267 | 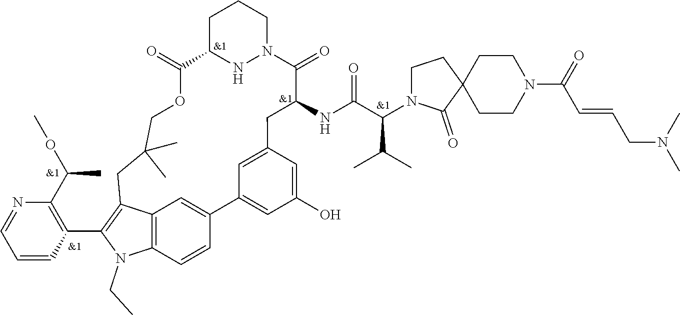 |
| A268 | 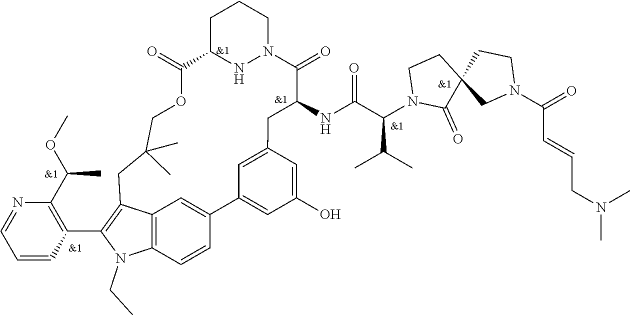 |
| A269 | 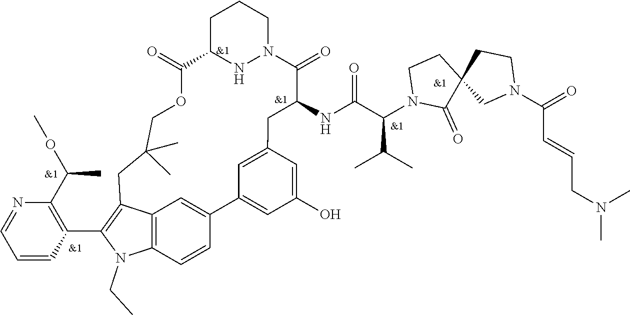 |
| A270 | 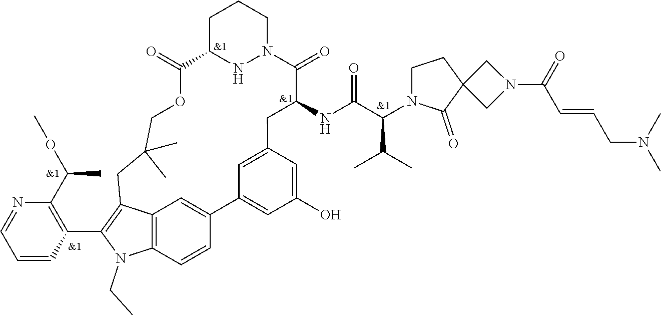 |
| A271 | 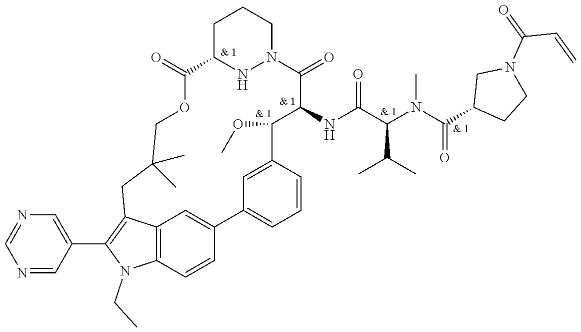 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@@H]2OC)C(C)C)C1] |
| A272 | 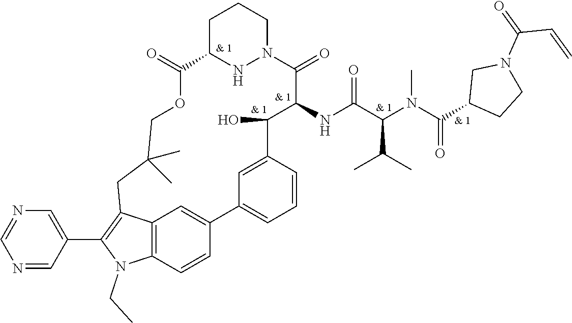 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2O)C(C)C)C1] |
| A273 | 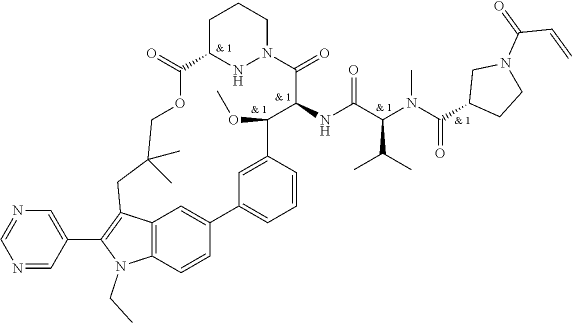 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2OC)C(C)C)C1] |
| A274 | 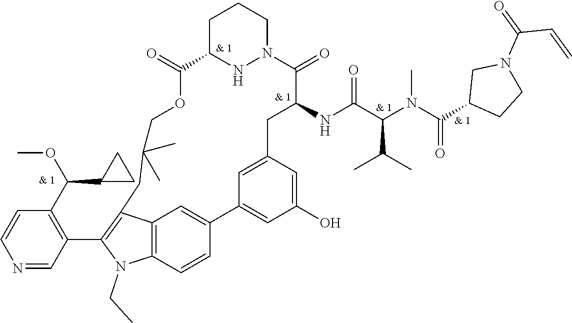 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@@H](OC)C3CC3)n4CC)CC(C)(C)COC(=O)[C@@H]3CCCN(N3)C2=O)C(C)C)C1] |
| A275 | 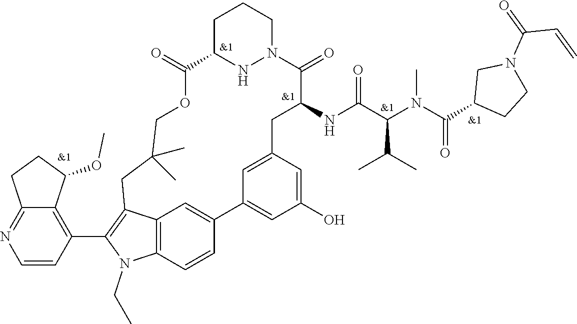 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CC5)n4CC)CC(C)(C)COC(=O)[C@@H]3CCCN(N3)C2=O)C(C)C)C1] |
| A276 | 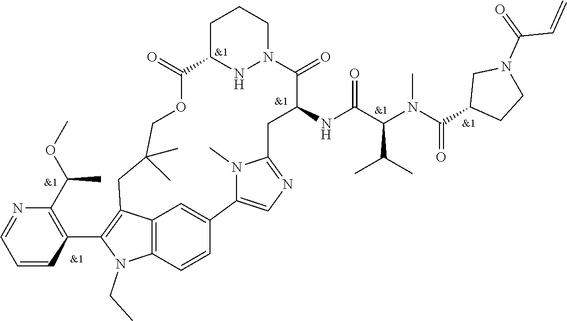 |
| A277 | 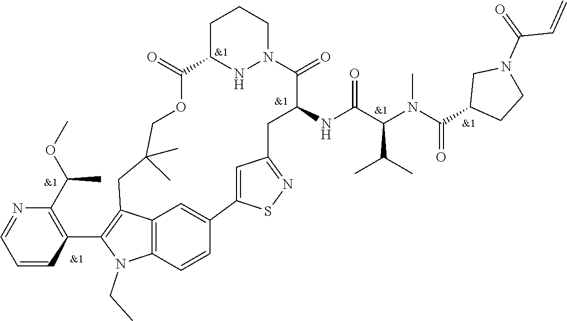 |
| A278 | 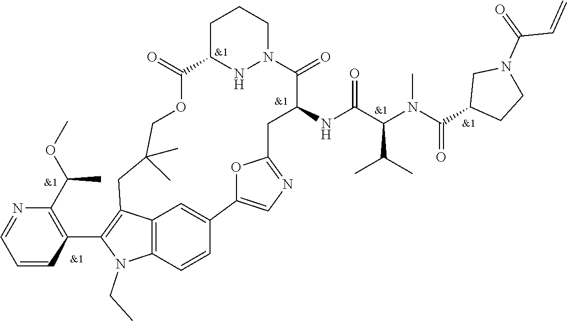 |
| A279 | 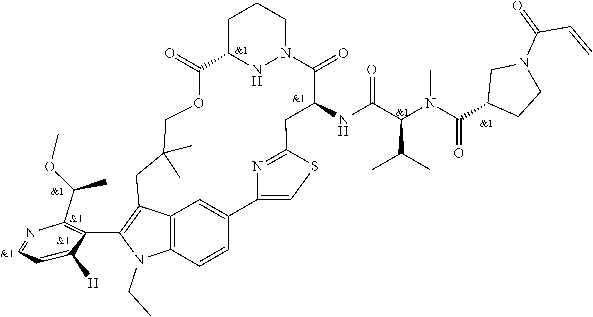 |
| A280 | 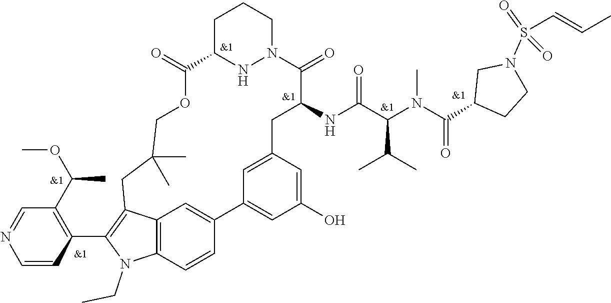 |
| A281 | 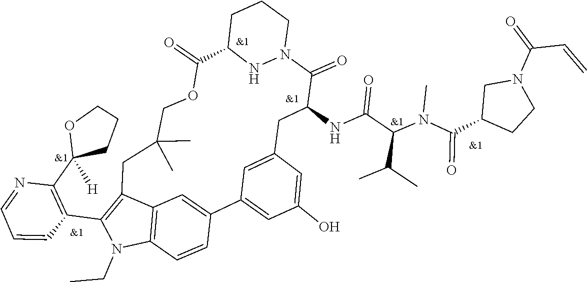 |
| A282 | 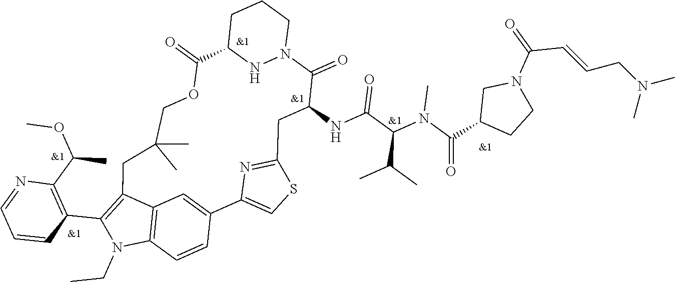 |
| A283 | 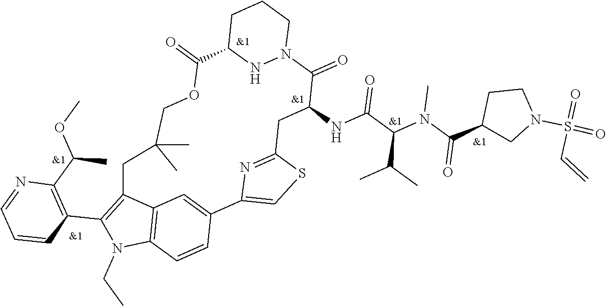 |
| A284 | 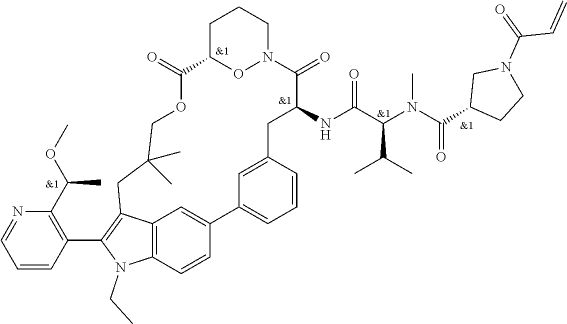 [SMILES: C=CC(=O)N1CC[C@H](C(=O)N(C)[C@H](C(=O)N[C@H]2Cc3cccc(c3)-c3ccc4c(c3)c(c(-c3cccnc3[C@H](C)OC)n4CC)CC(C)(C)COC(=O)[C@@H]3CCCN(O3)C2=O)C(C)C)C1] |
| A285 | 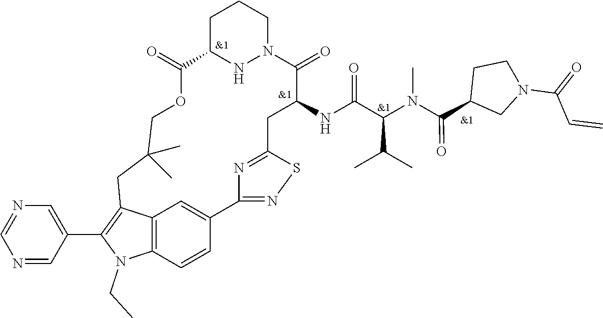 [SMILES: C=CC(=O)N1CC[C@H](C(=O)N(C)[C@H](C(=O)N[C@H]2Cc3nc(ns3)-c3ccc4c(c3)c(c(-c3cncnc3)n4CC)CC(C)(C)COC(=O)[C@@H]3CCCN(N3)C2=O)C(C)C)C1] |
| A286 | 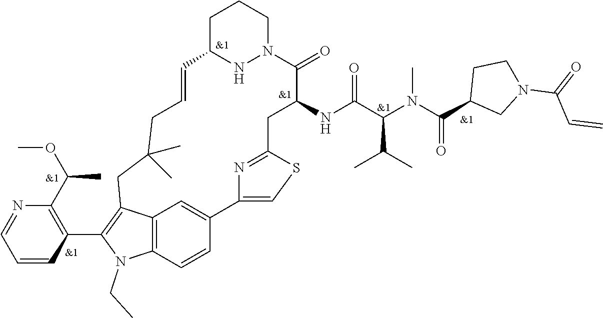 |
| A287 | 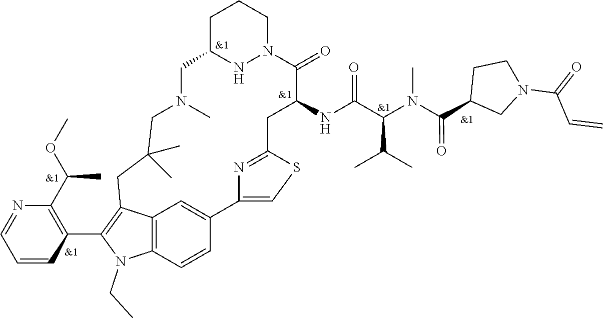 |
| A288 | 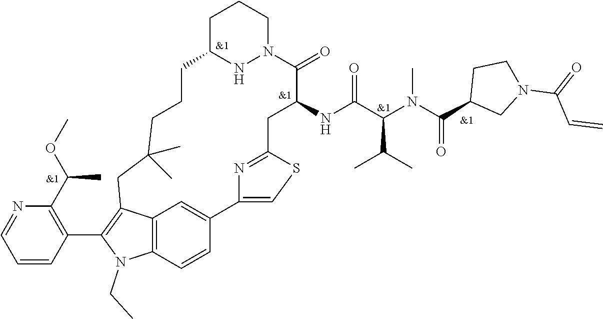 [SMILES: C=CC(=O)N1CC[C@H](C(=O)N(C)[C@H](C(=O)N[C@H]2Cc3nc(cs3)-c3ccc4c(c3)c(c(-c3cccnc3[C@H](C)OC)n4CC)CC(C)(C)CCC[C@@H]3CCCN(N3)C2=O)C(C)C)C1] |
| A289 | 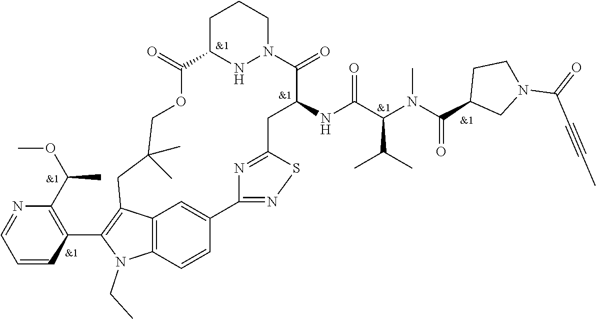 |
| A290 | 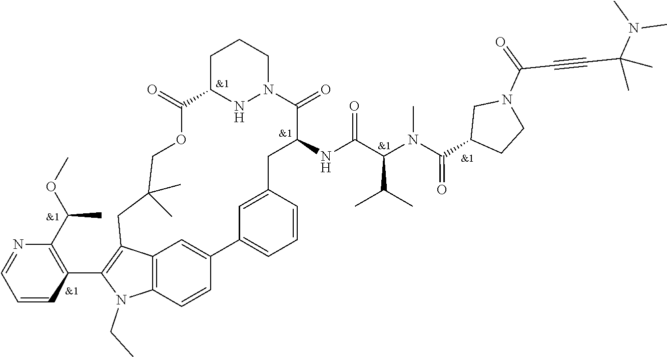 |
| A291 | 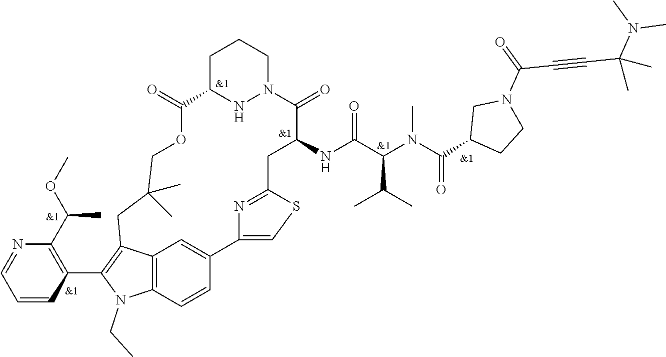 |
| A292 | 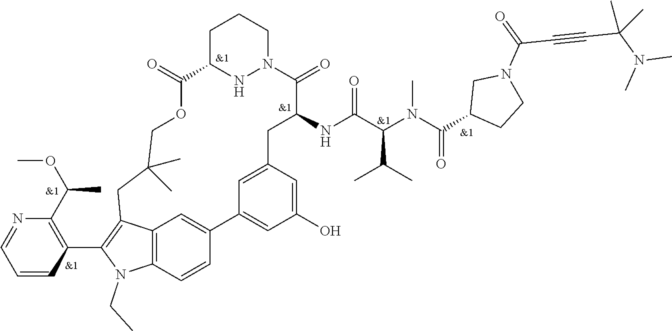 |
| A293 | 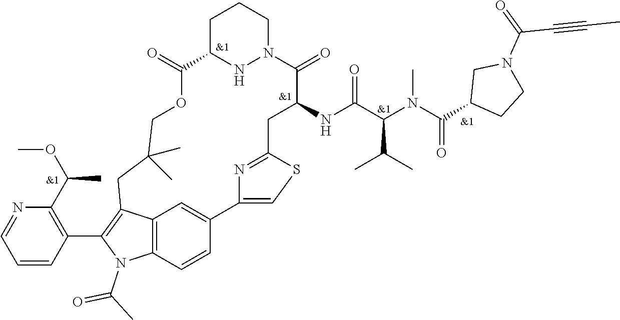 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(C)=O)CC(C)(C)COC(=O)[C@@H]3CCCN(N3)C2=O)C(C)C)C1] |
| A294 | 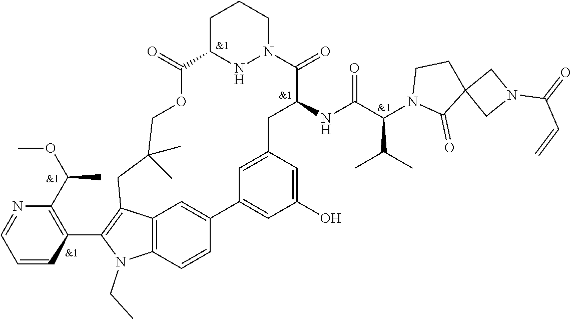 |
| A295 | 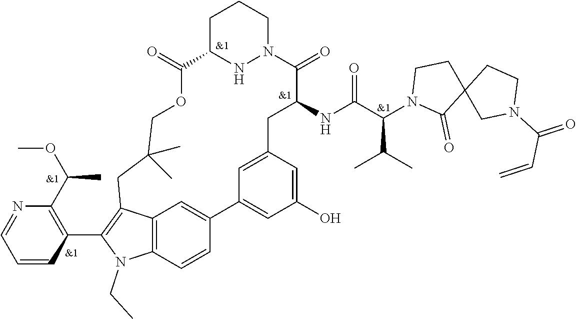 |
| A296 | 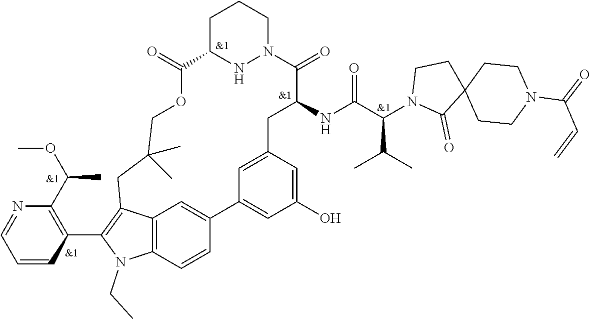 |
| A297 | 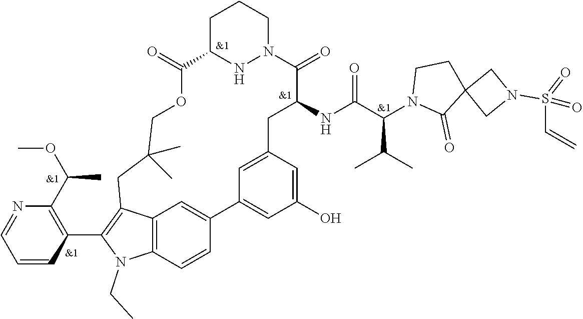 |
| A298 | 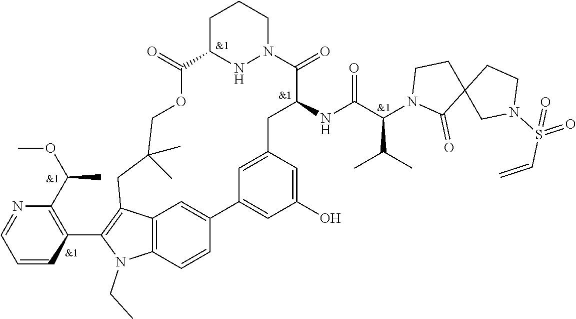 |
| A299 | 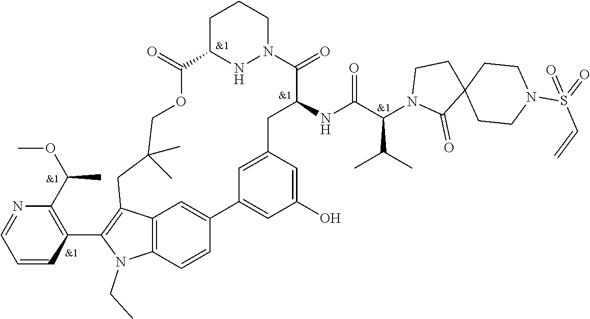 |
| A300 | 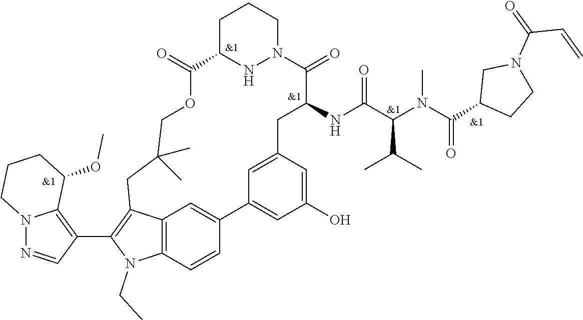 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| A301 | 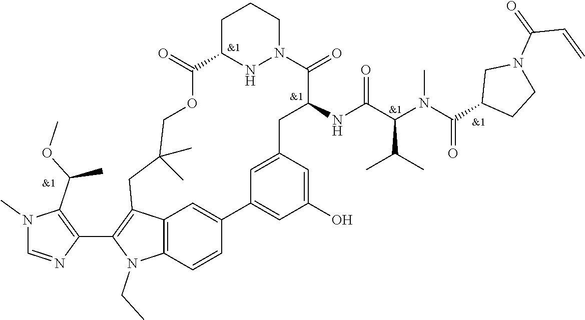 [SMILES: C=CC(=O)N1CC[C@H](C(=O)N(C)[C@H](C(=O)N[C@H]2Cc3cc(O)cc(c3)-c3ccc4c(c3)c(c(-c3ncn(C)c3[C@H](C)OC)n4CC)CC(C)(C)COC(=O)[C@@H]3CCCN(N3)C2=O)C(C)C)C1] |
| A302 | 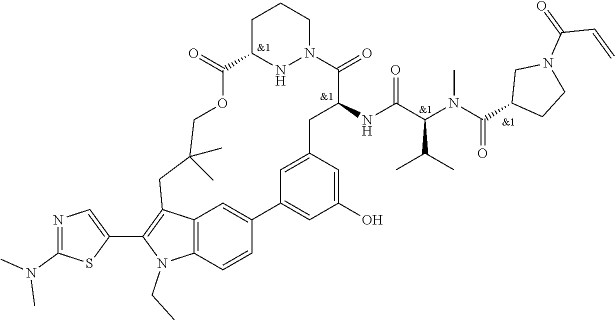 [SMILES: C=CC(=O)N1CC[C@H](C(=O)N(C)[C@H](C(=O)N[C@H]2Cc3cc(O)cc(c3)-c3ccc4c(c3)c(c(-c3cnc(N(C)C)s3)n4CC)CC(C)(C)COC(=O)[C@@H]3CCCN(N3)C2=O)C(C)C)C1] |
| A303 | 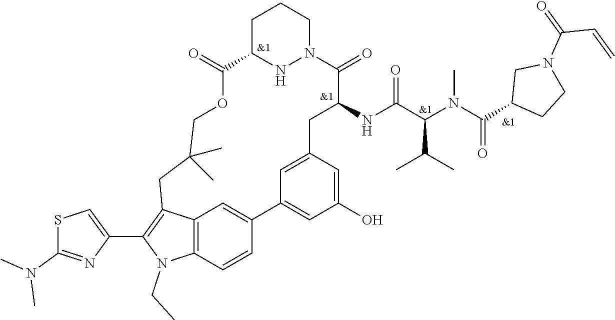 [SMILES: C=CC(=O)N1CC[C@H](C(=O)N(C)[C@H](C(=O)N[C@H]2Cc3cc(O)cc(c3)-c3ccc4c(c3)c(c(-c3csc(N(C)C)n3)n4CC)CC(C)(C)COC(=O)[C@@H]3CCCN(N3)C2=O)C(C)C)C1] |
| A304 | 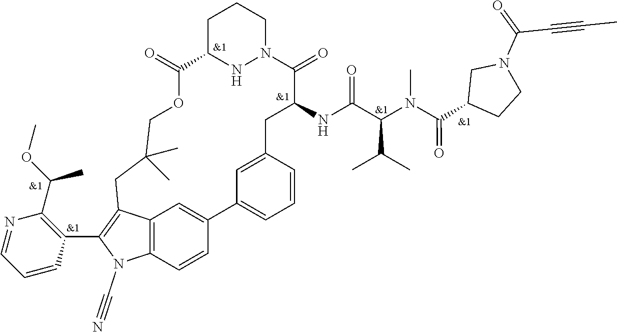 |
| A305 | 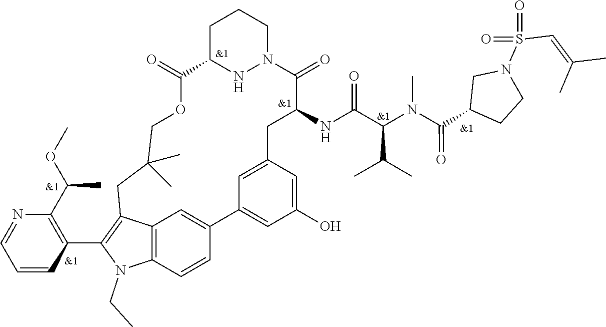 |
| A306 | 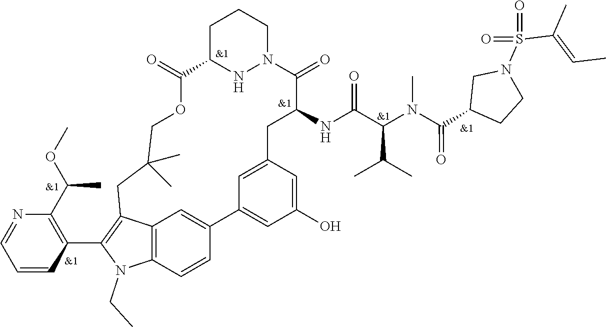 |
| A307 | 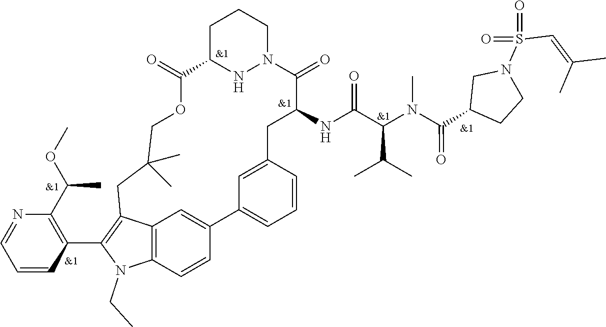 |
| A308 | 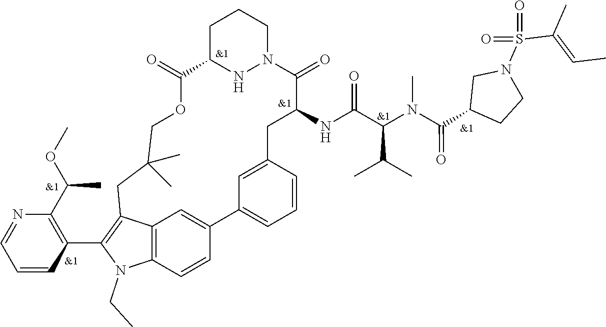 |
| A309 | 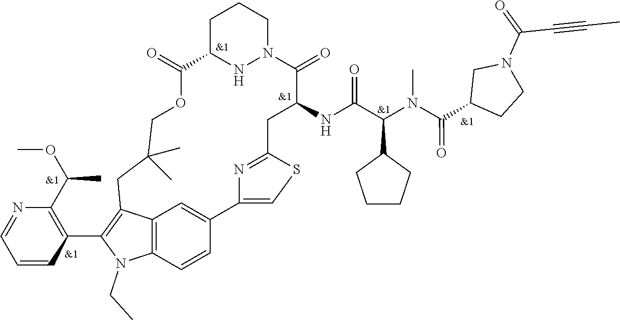 |
| A310 | 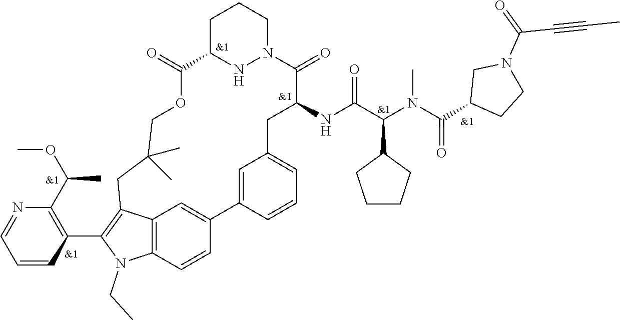 |
| A311 | 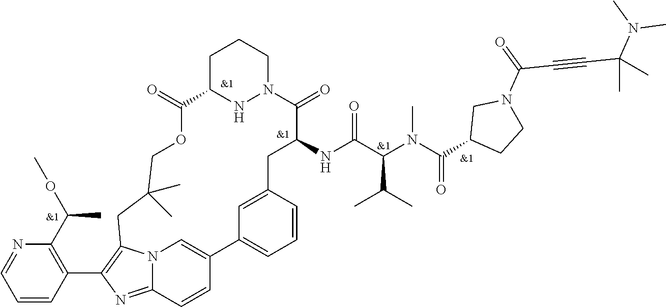 [SMILES: CO[C@@H](C)c1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CC(C)(C)N(C)C)C1)Cc1cccc-3c1] |
| A312 | 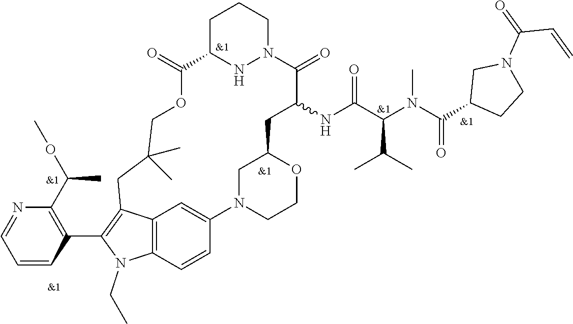 |
| A313 | 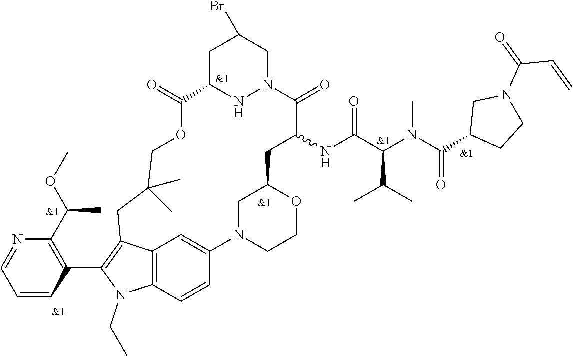 |
| A314 | 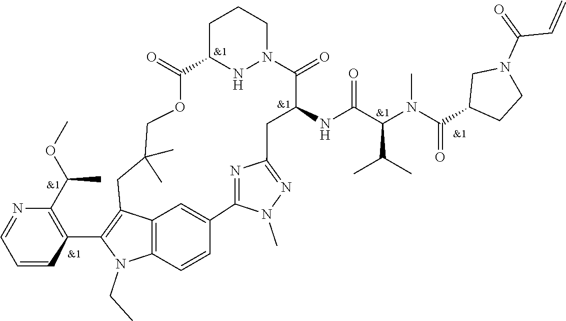 |
| A316 | 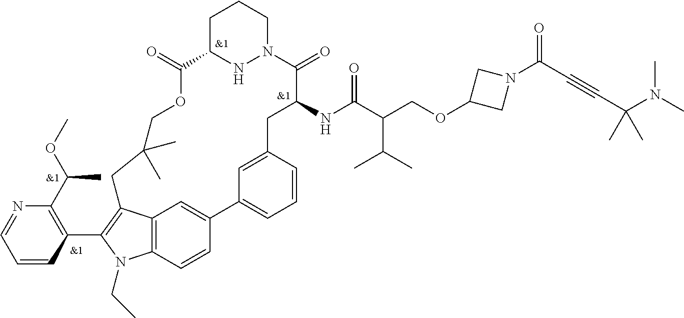 |
| A317 | 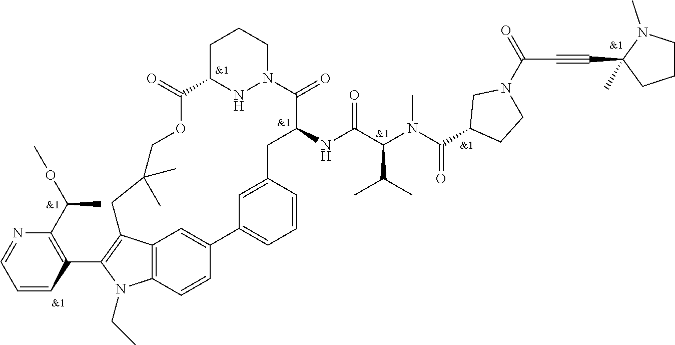 |
| A318 | 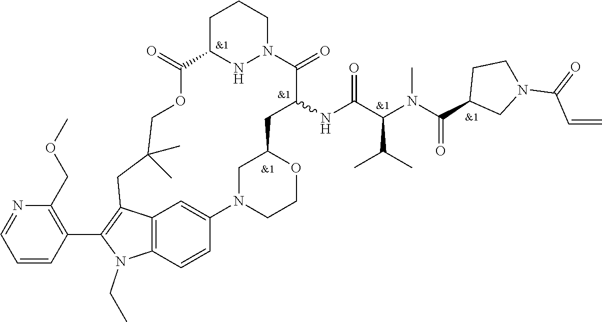 [SMILES: C=CC(=O)N1CC[C@H](C(=O)N(C)[C@H](C(=O)NC2C[C@@H]3CN(CCO3)c3ccc4c(c3)c(c(-c3cccnc3COC)n4CC)CC(C)(C)COC(=O)[C@@H]3CCCN(N3)C2=O)C(C)C)C1] |
| A319 | 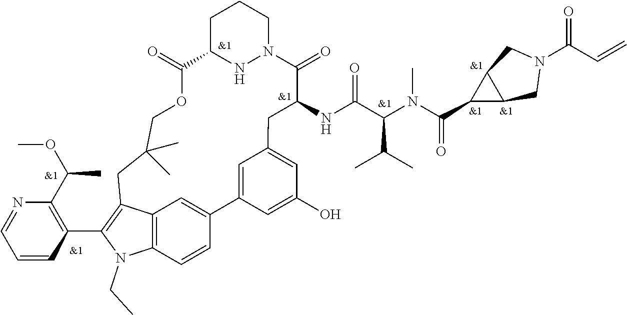 |
| A320 | 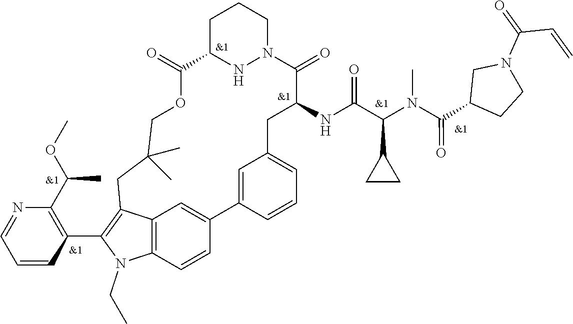 |
| A321 | 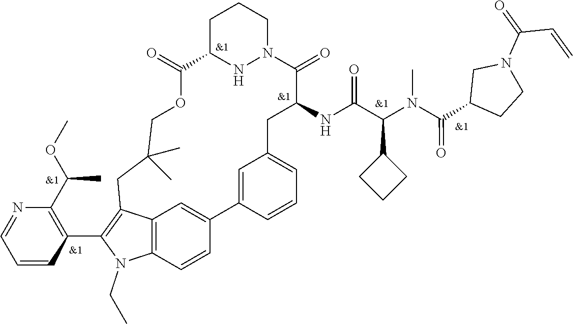 |
| A322 | 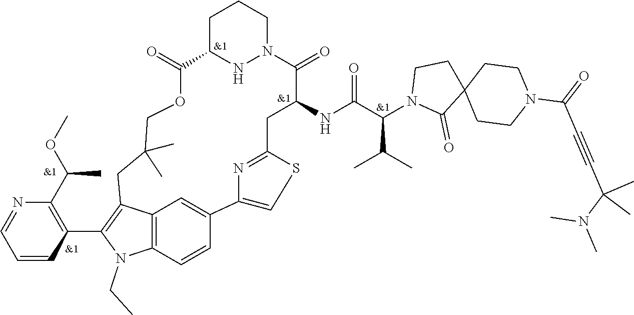 |
| A323 | 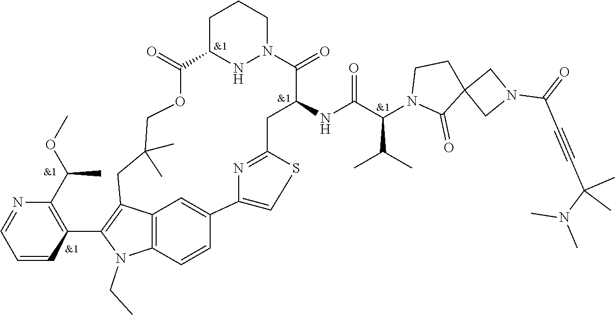 |
| A324 | 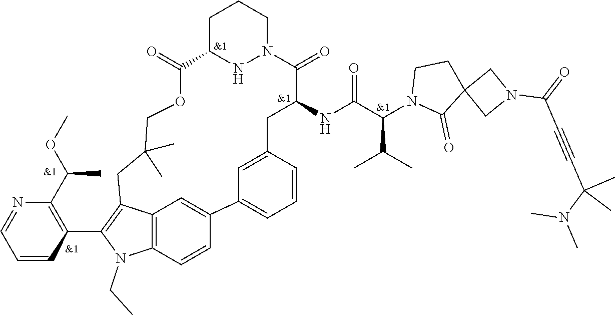 |
| A325 | 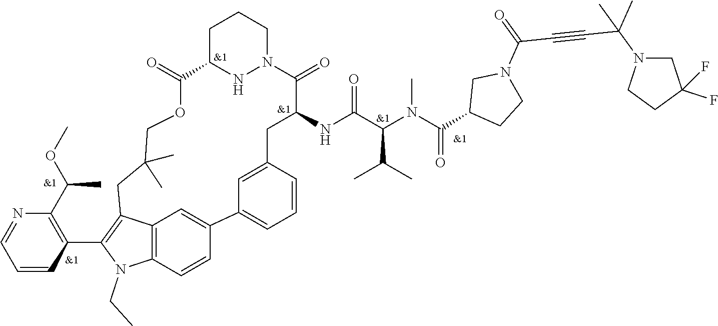 |
| A326 | 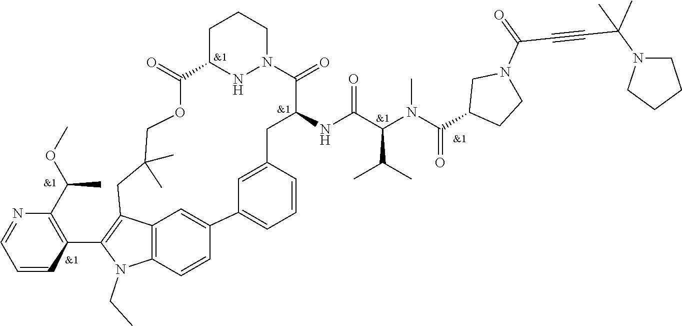 |
| A327 | 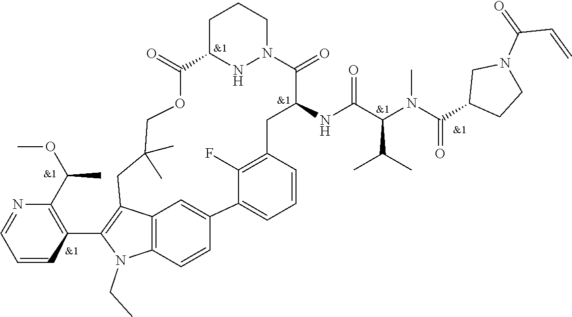 |
| A328 | 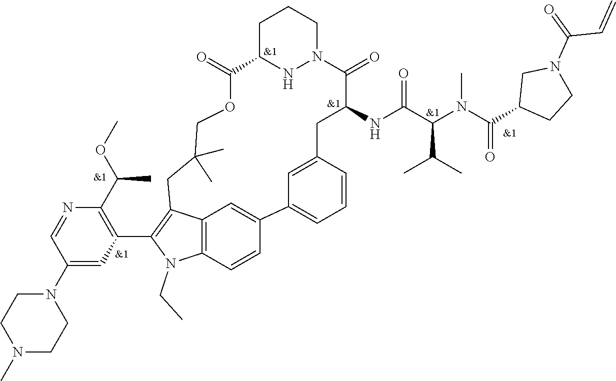 |
| A329 | 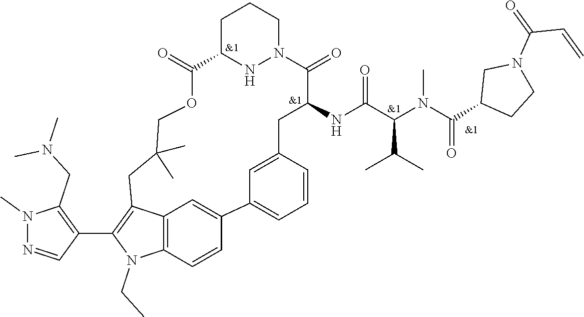 [SMILES: C=CC(=O)N1CC[C@H](C(=O)N(C)[C@H](C(=O)N[C@H]2Cc3cccc(c3)-c3ccc4c(c3)c(c(-c3cnn(C)c3CN(C)C)n4CC)CC(C)(C)COC(=O)[C@@H]3CCCN(N3)C2=O)C(C)C)C1] |
| A330 | 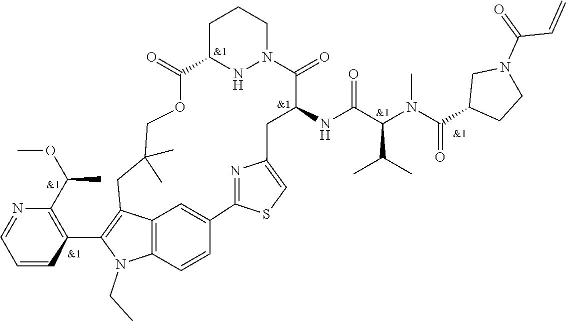 |
| A331 | 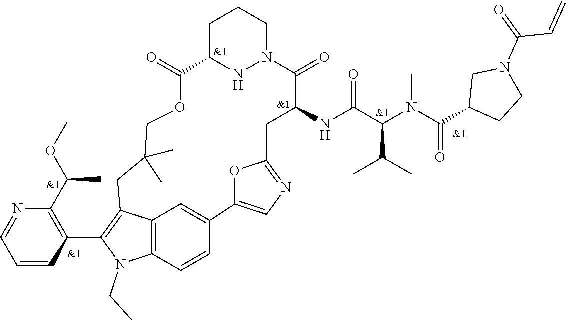 |
| A332 | 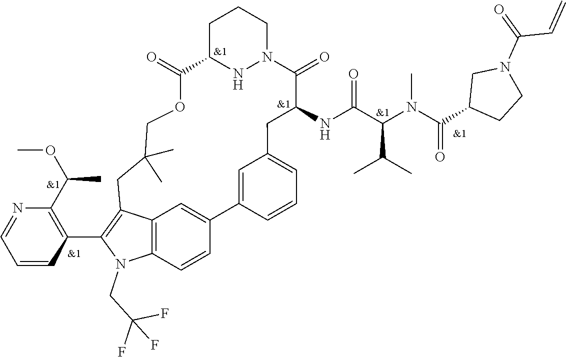 |
| A333 | 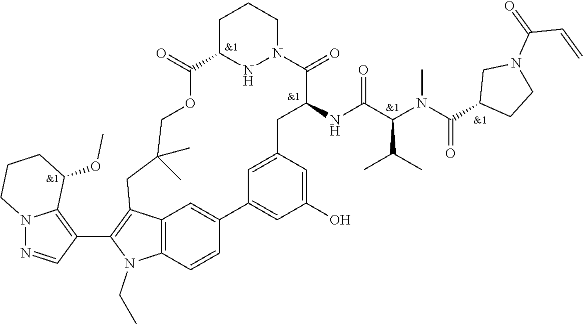 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| A334 | 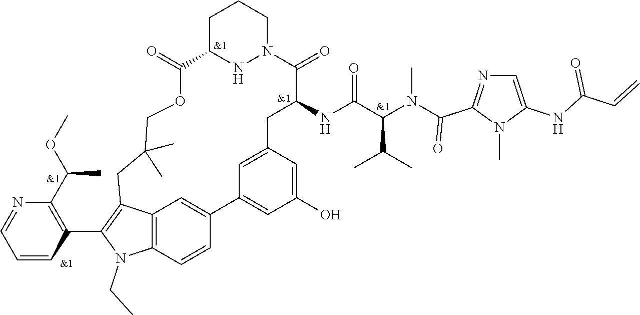 |
| A335 | 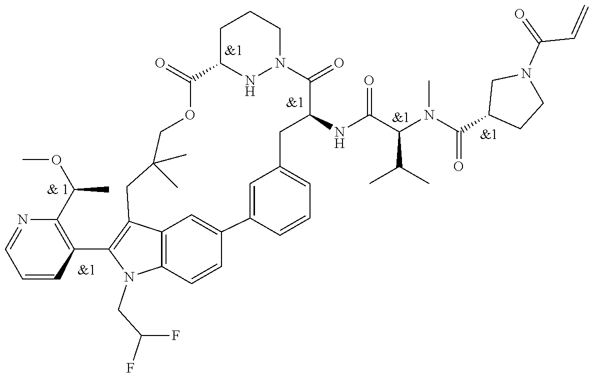 |
| A336 | 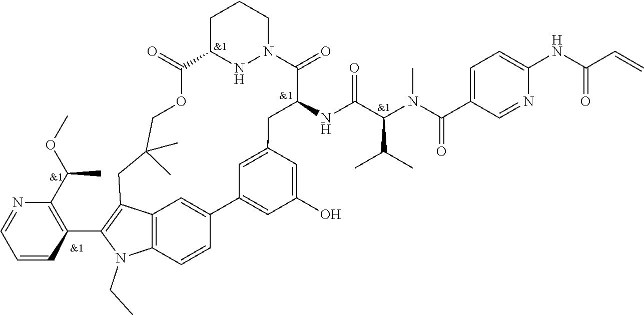 |
| A337 | 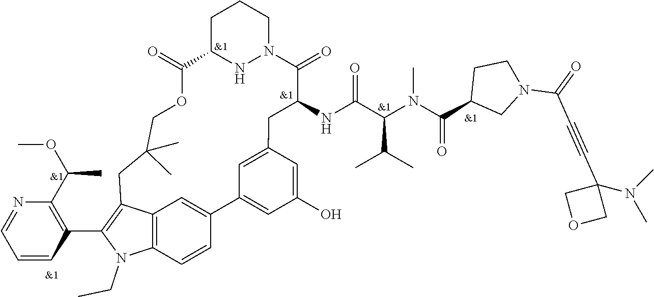 |
| A338 | 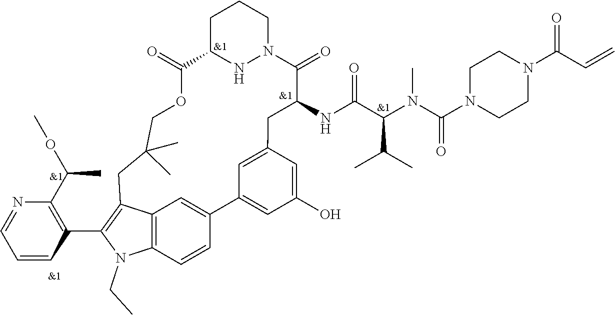 |
| A339 | 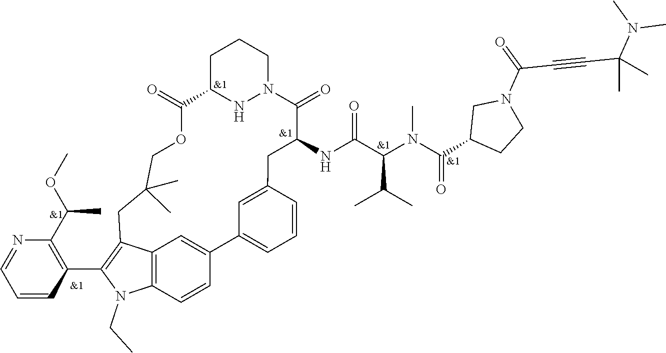 |
| A340 | 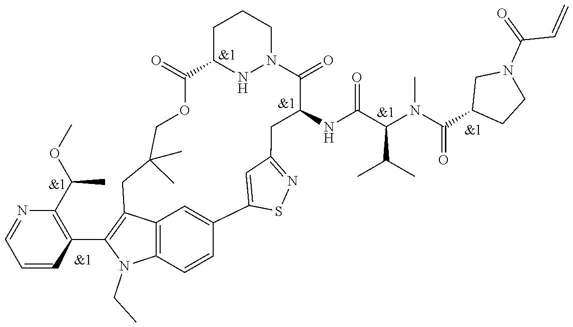 |
| A341 | 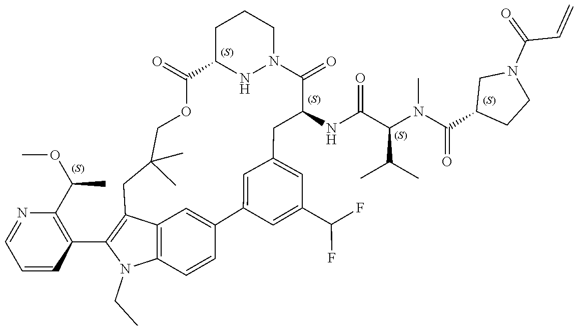 |
| A342 | 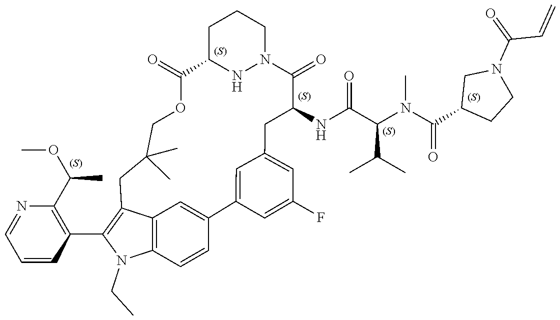 |
| A343 | 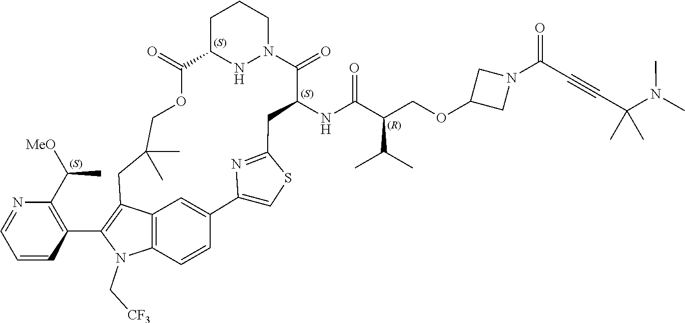 |
| A344 | 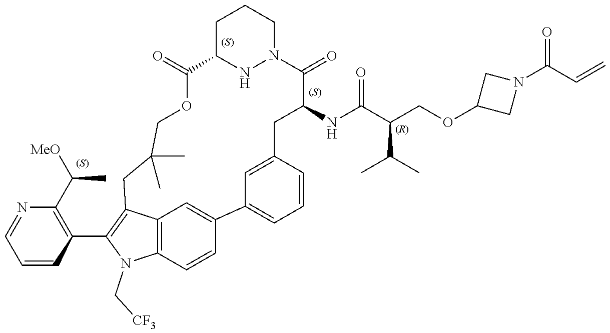 |
| A345 | 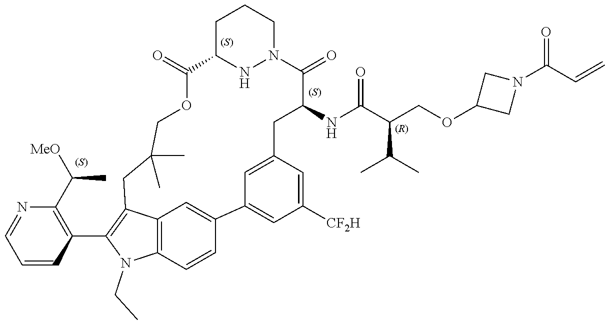 |
| A346 | 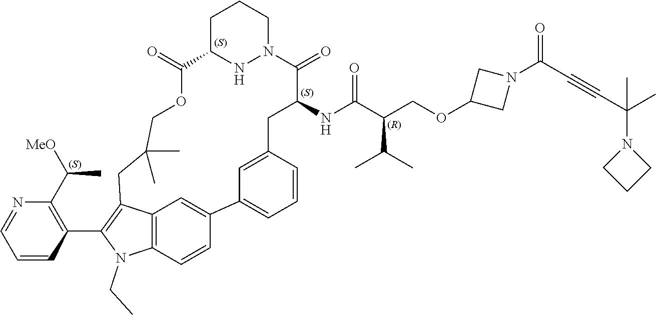 |
| A347 | 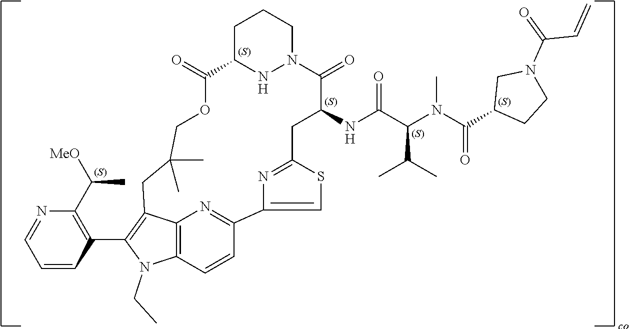 |
| A348 | 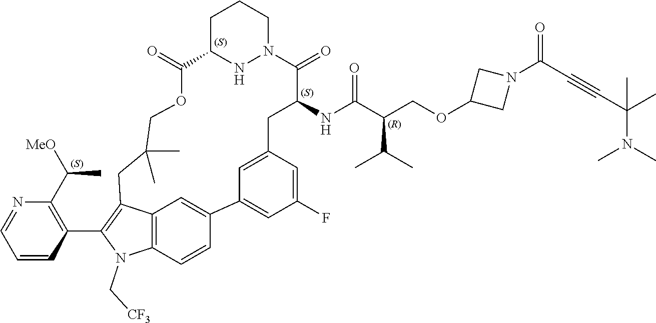 |
| A349 | 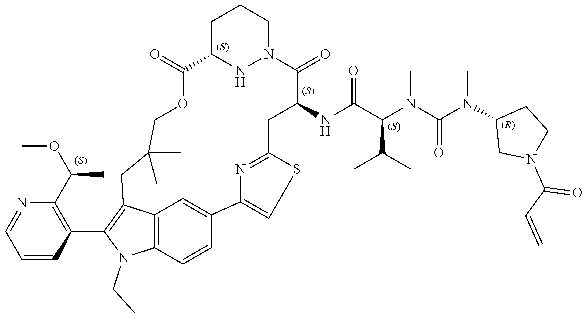 |
| A350 | 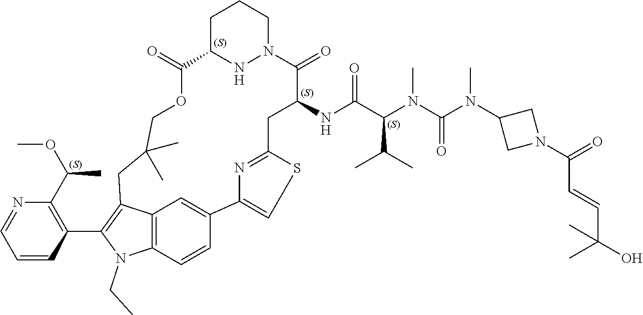 |
| A351 | 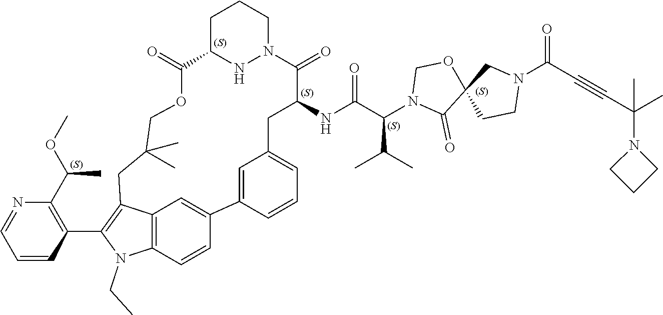 |
| A352 | 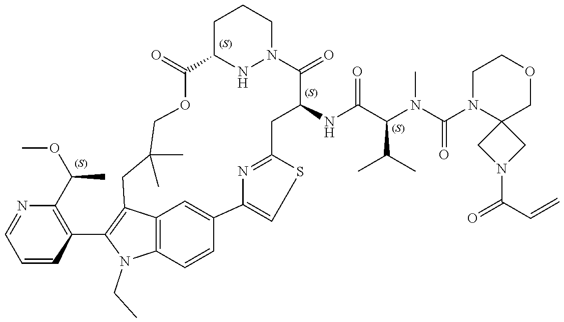 |
| A353 | 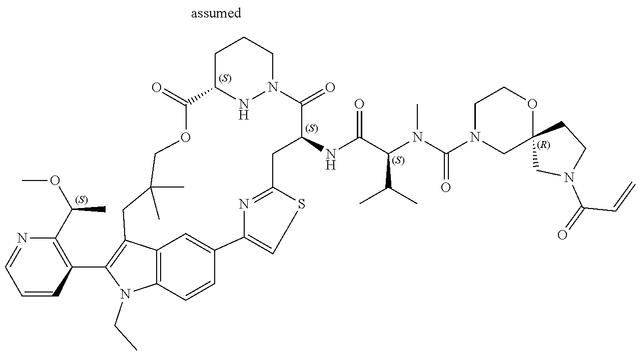 |
| A354 | 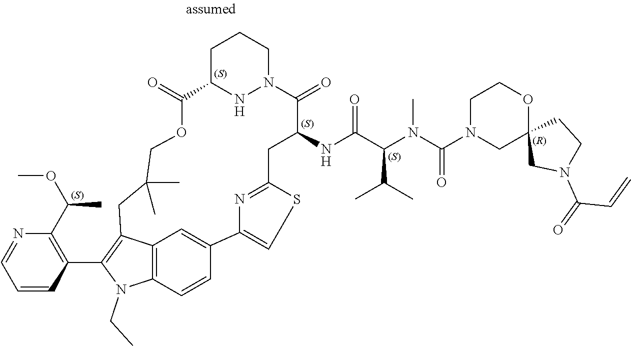 |
| A355 | 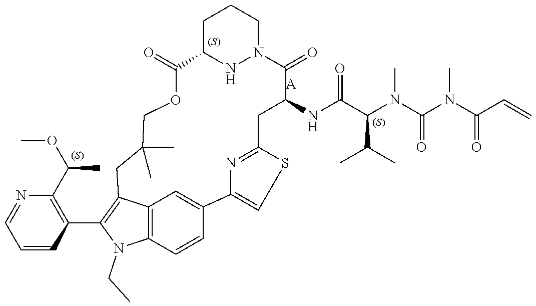 |
| A356 | 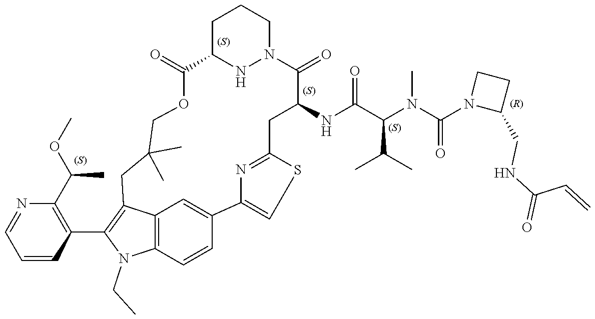 |
| A357 | 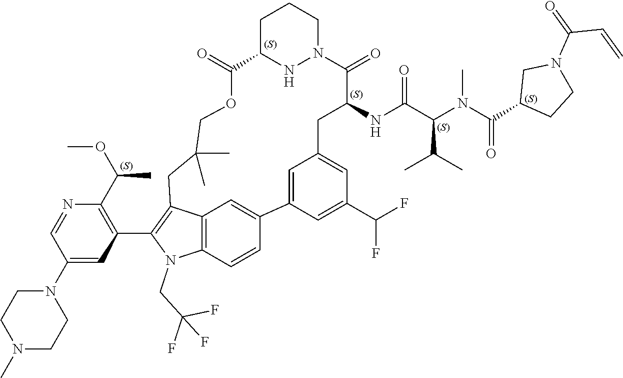 |
| A358 | 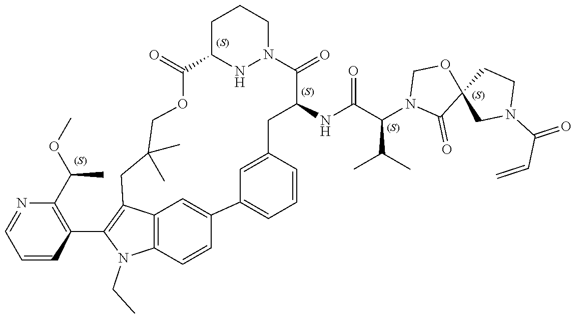 |
| A359 | 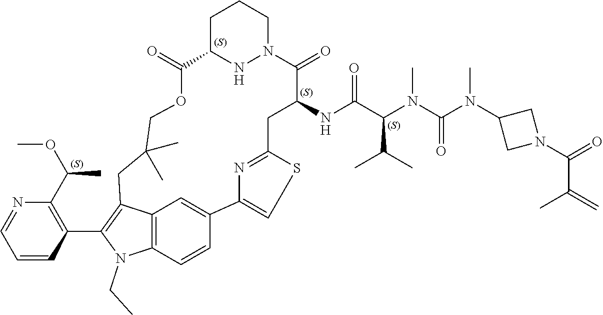 |
| A360 | 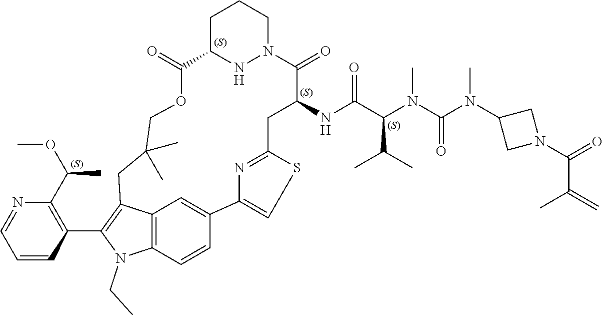 |
| A361 | 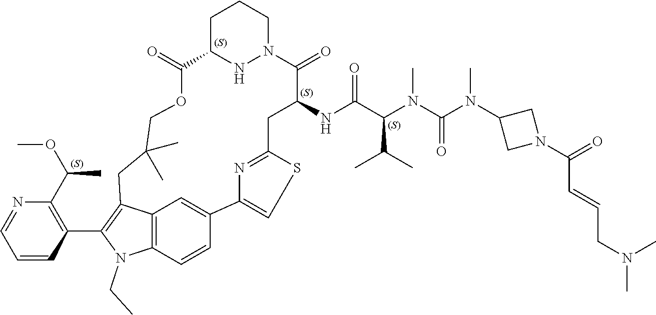 |
| A362 | 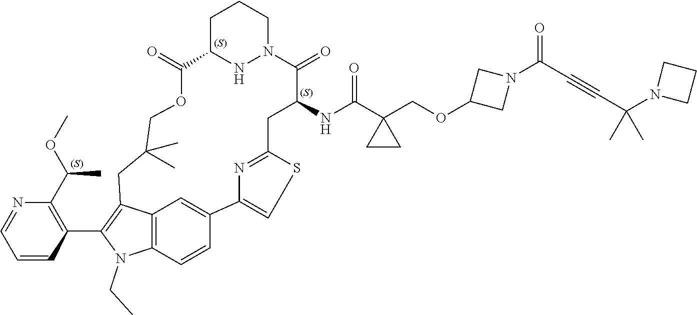 |
| A363 | 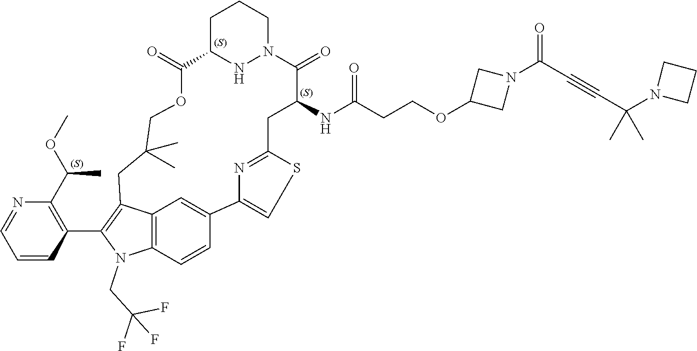 |
| A364 | 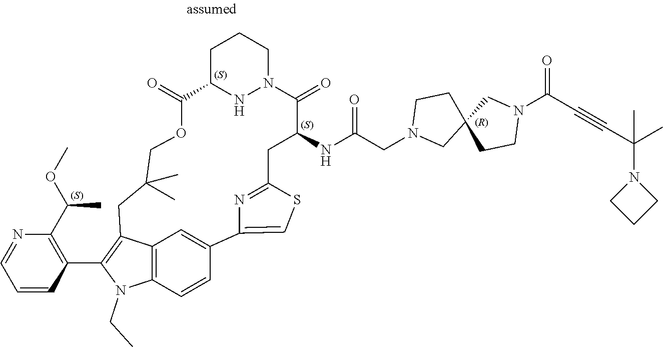 |
| A365 | 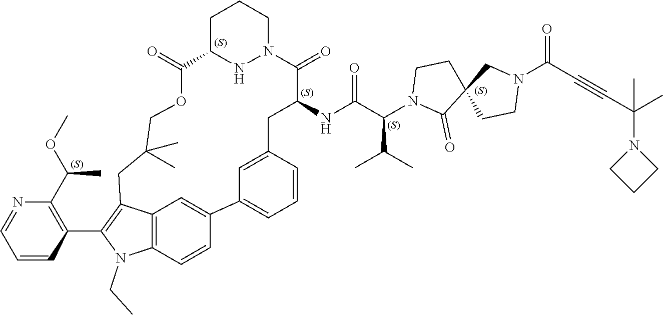 |
| A366 | 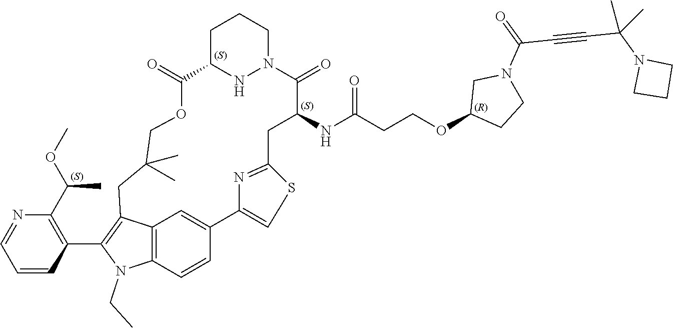 |
| A367 | 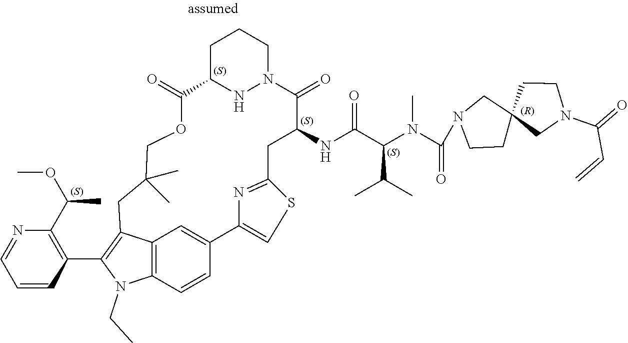 |
| A368 | 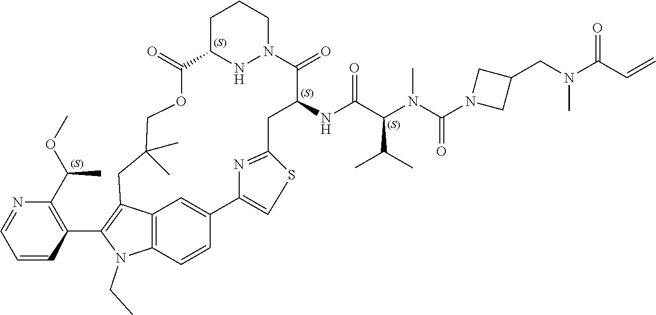 |
| A369 | 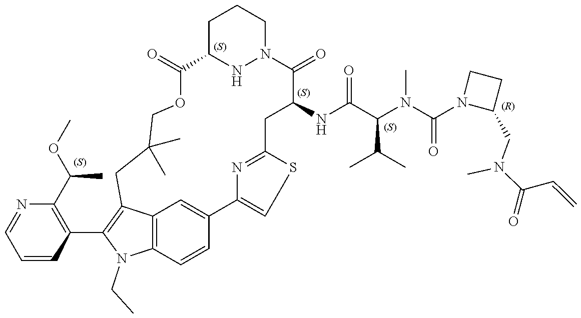 |
| A370 | 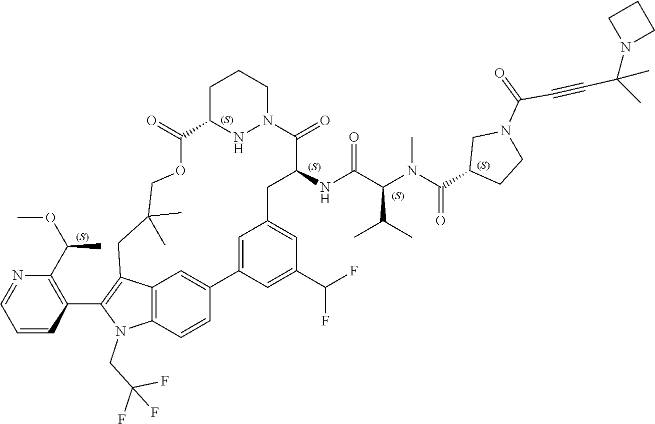 |
| A371 | 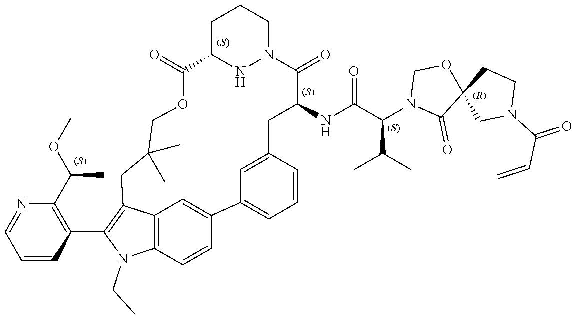 |
| A372 | 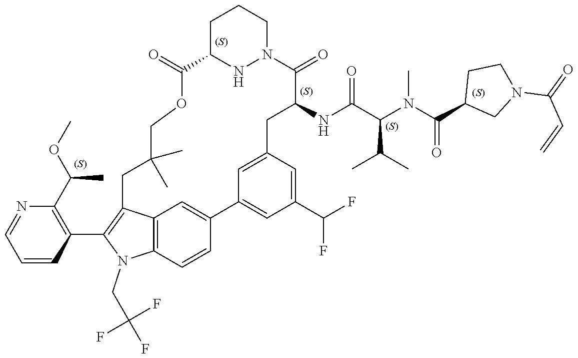 |
| A373 | 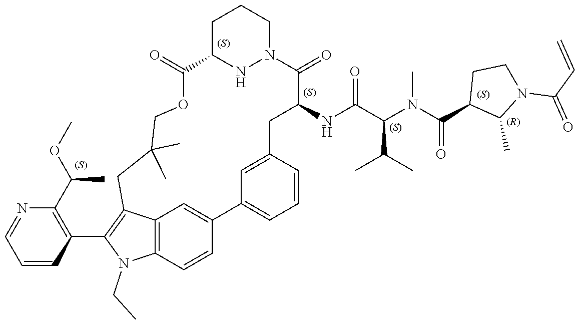 |
| A374 | 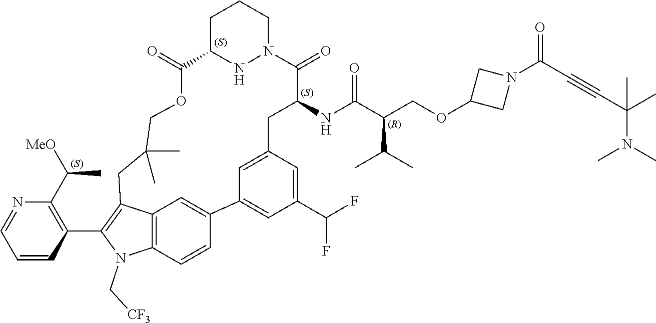 |
| A375 | 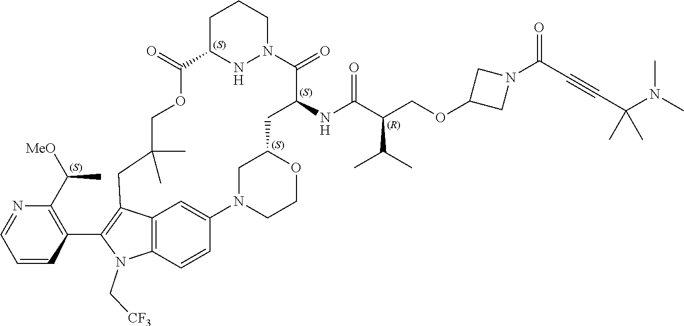 |
| A376 | 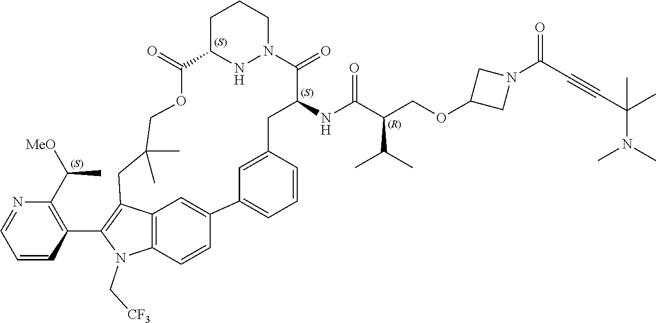 |
| A377 | 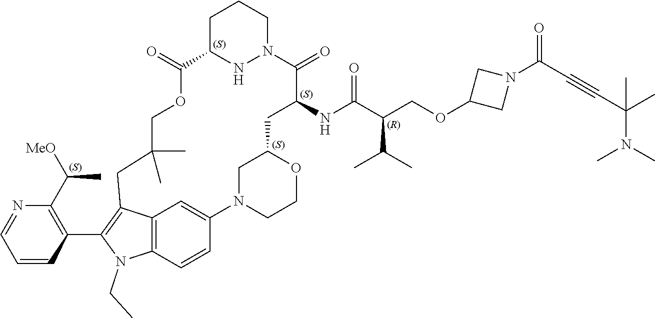 |
| A378 | 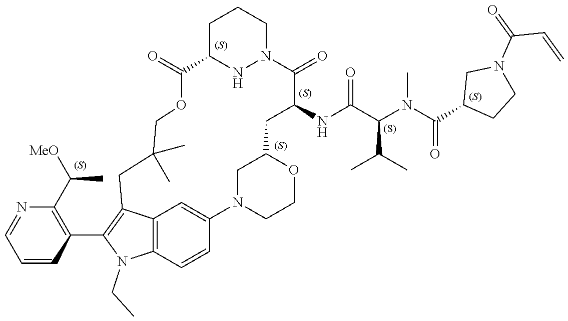 |
| A379 | 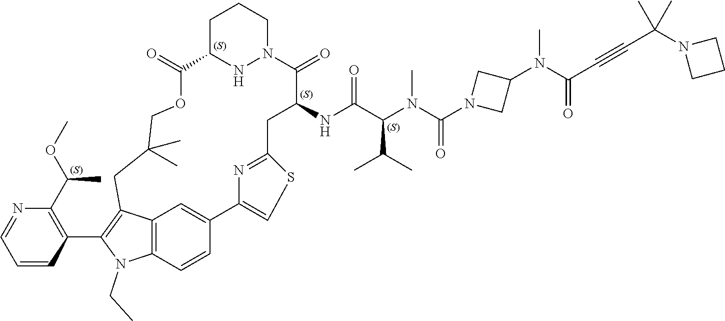 |
| A380 | 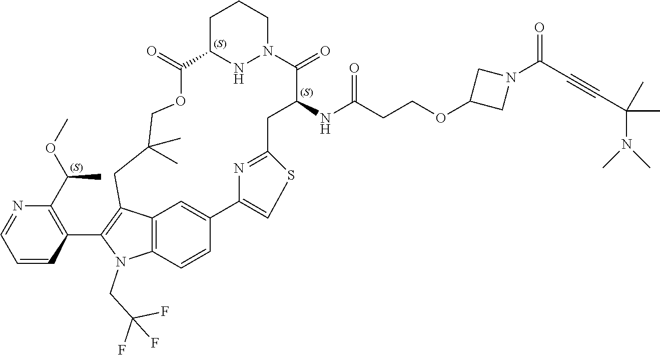 |
| A381 | 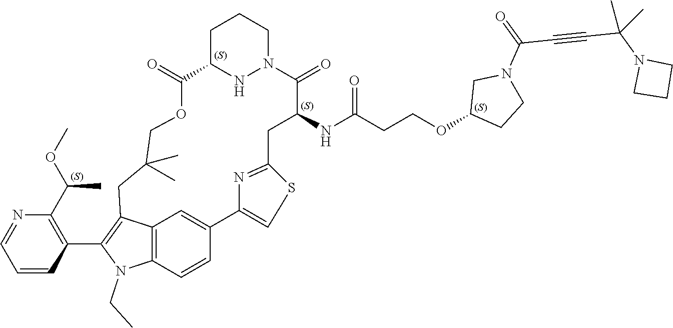 |
| A382 | 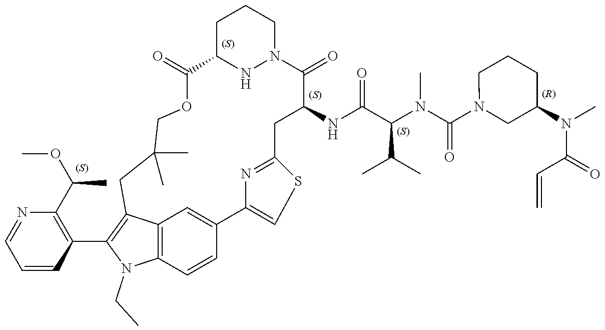 |
| A383 | 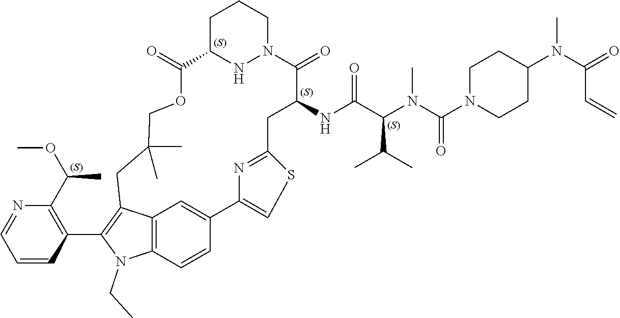 |
| A384 | 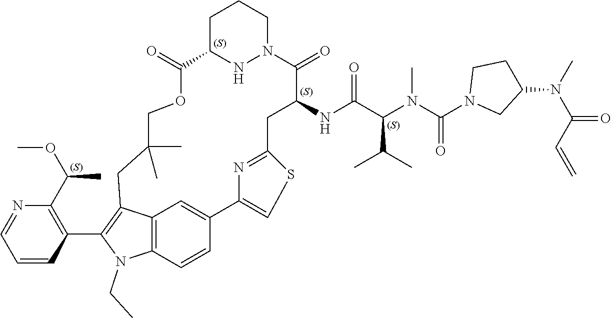 |
| A385 | 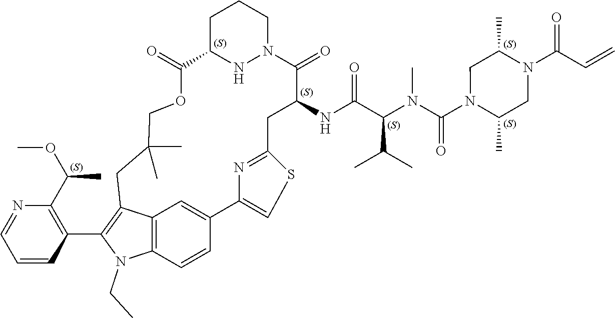 |
| A386 | 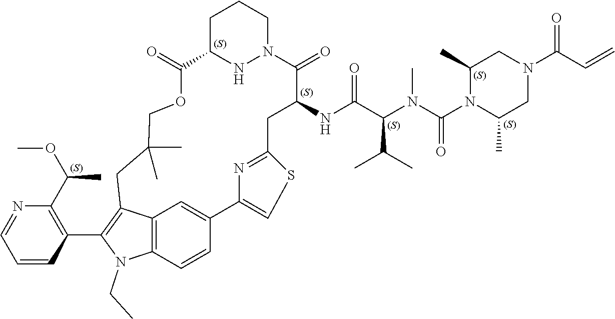 |
| A387 | 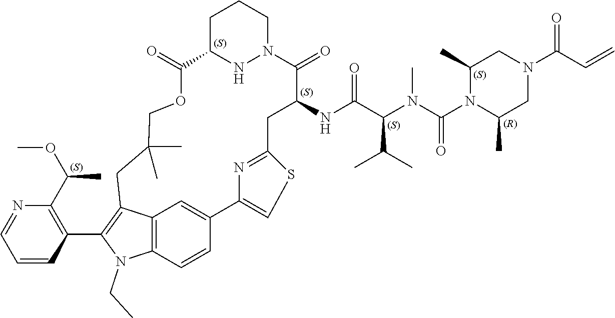 |
| A388 | 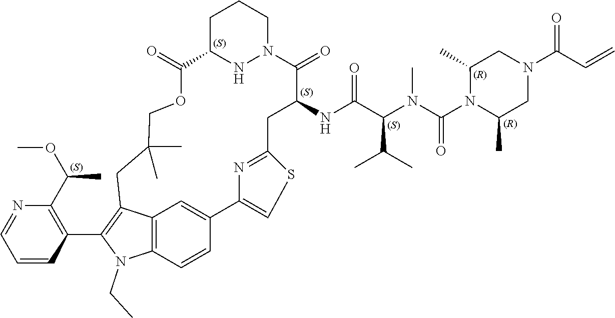 |
| A389 | 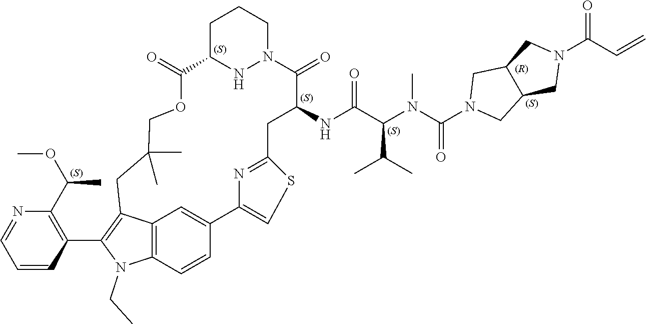 |
| A390 | 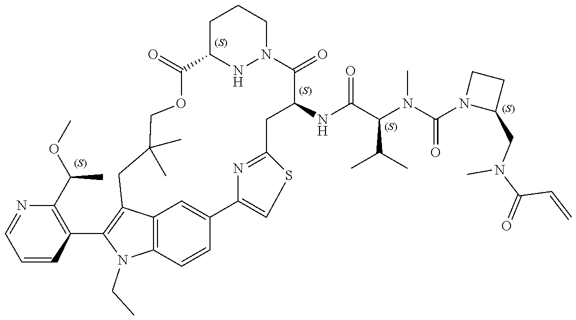 |
| A391 | 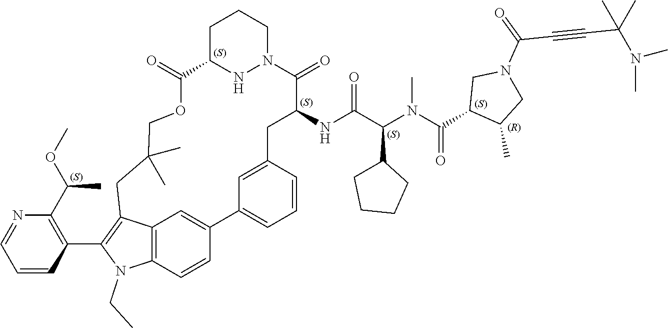 |
| A392 | 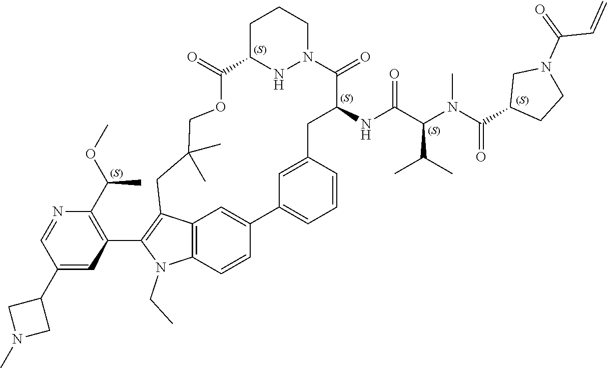 |
| A393 | 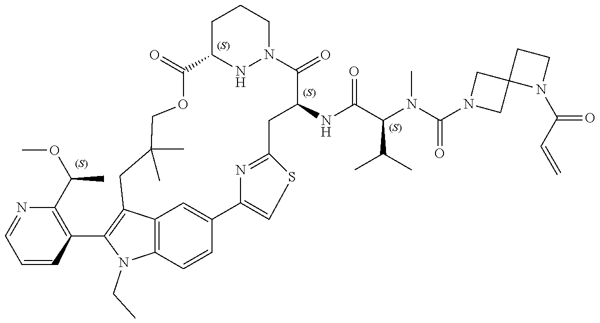 |
| A394 | 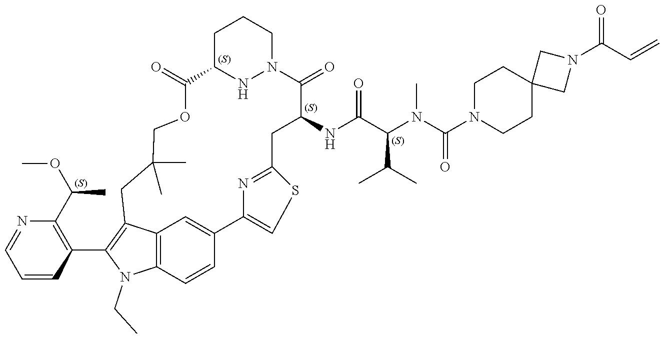 |
| A395 | 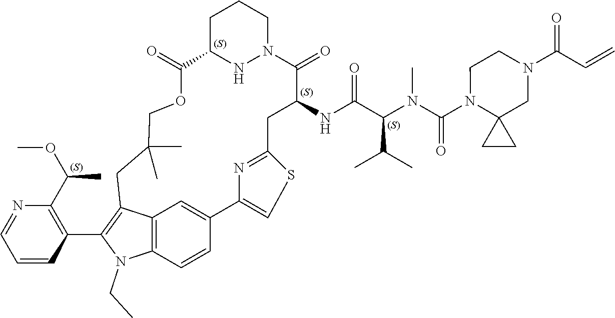 |
| A396 | 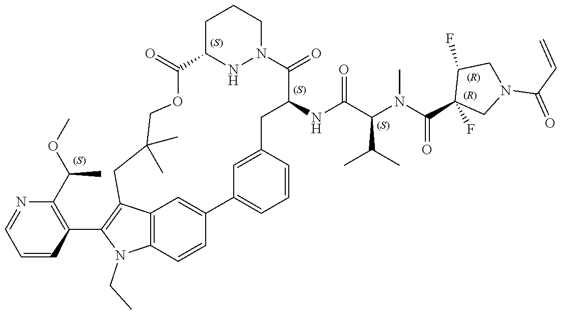 |
| A397 | 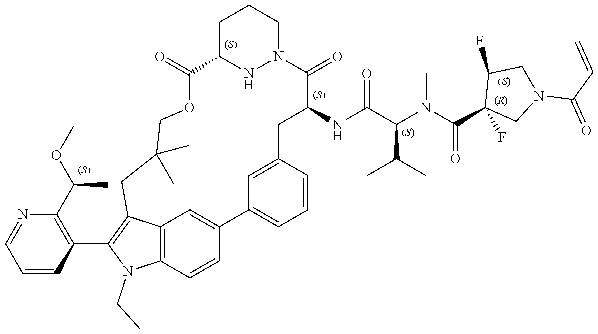 |
| A398 | 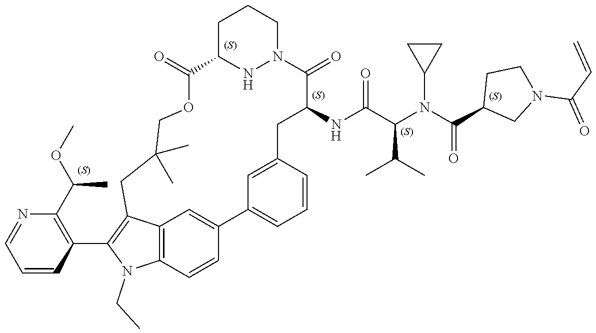 |
| A399 | 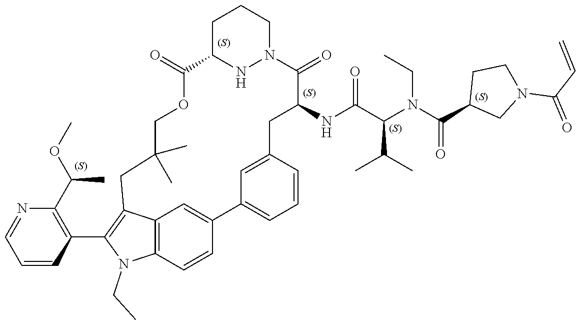 |
| A400 | 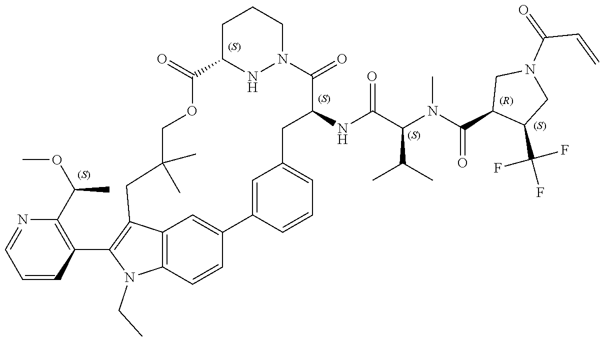 |
| A401 | 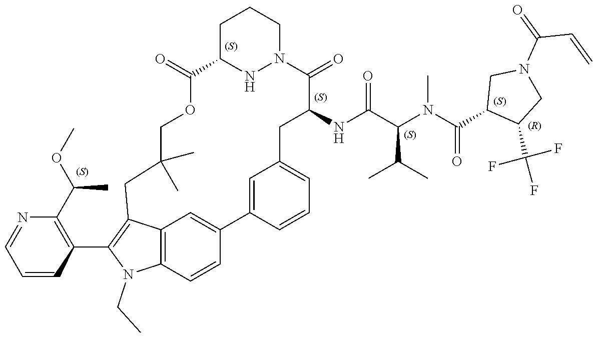 |
| A402 | 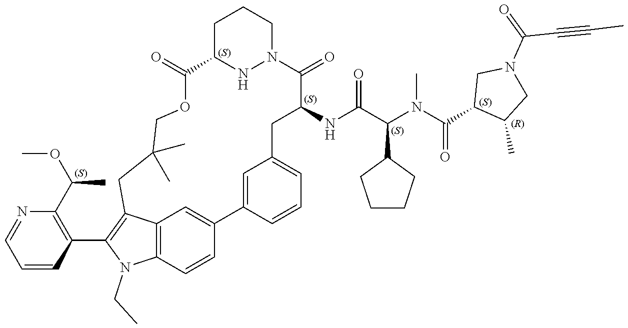 |
| A403 | 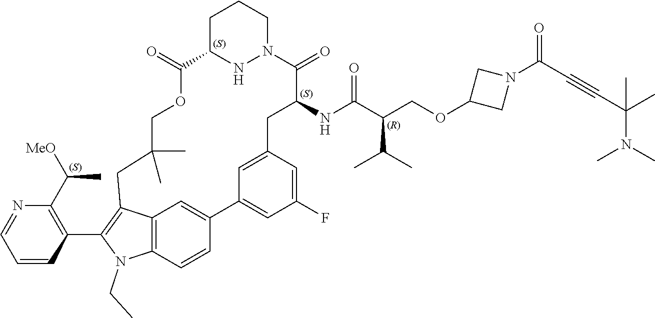 |
| A404 | 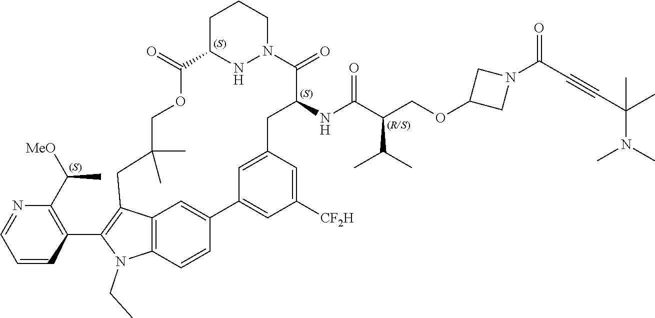 |
| A405 | 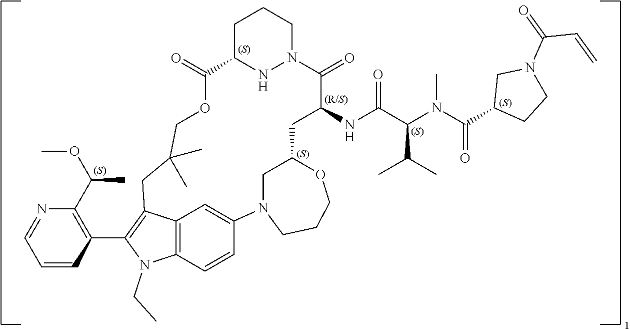 |
| A406 | 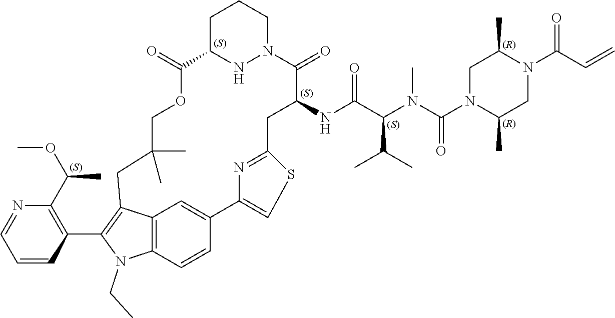 |
| A407 | 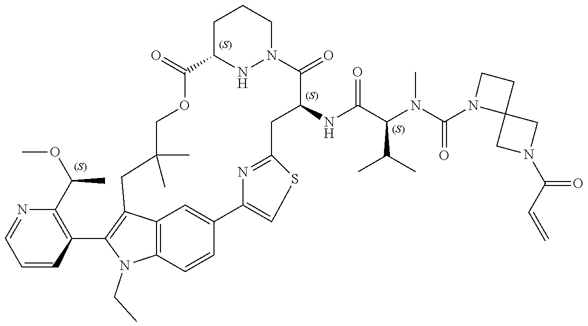 |
| A408 | 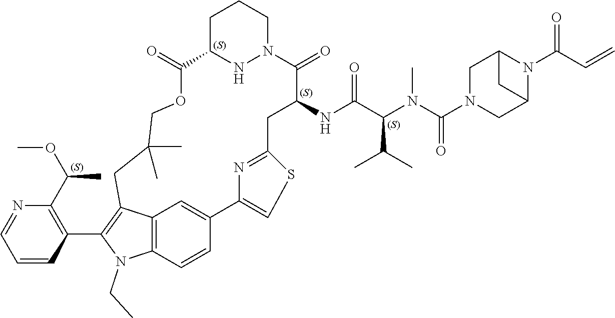 |
| A409 | 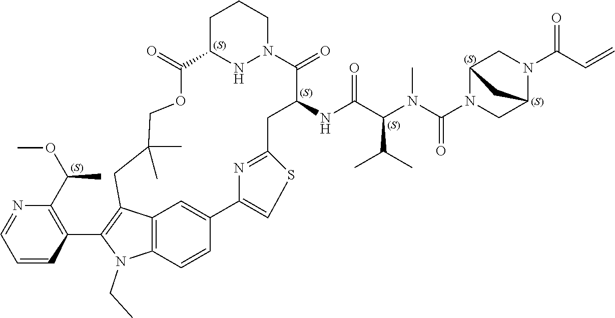 |
| A410 | 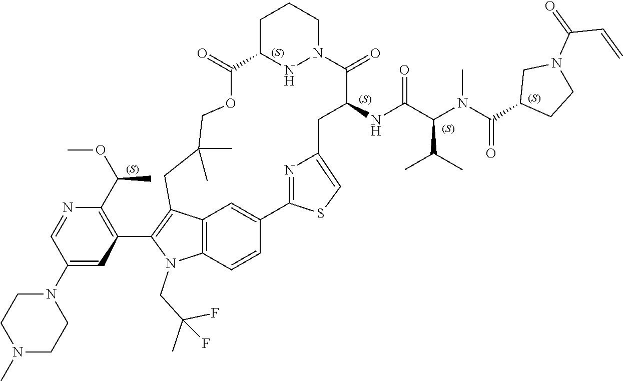 |
| A411 | 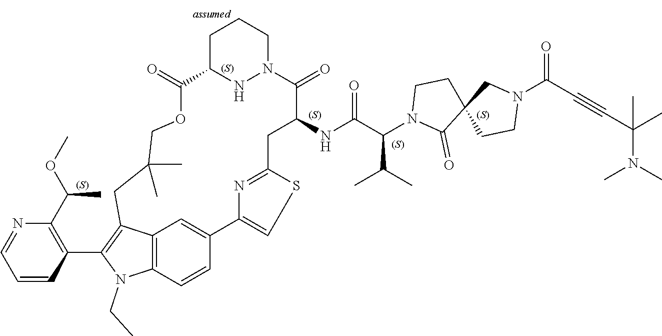 |
| A412 | 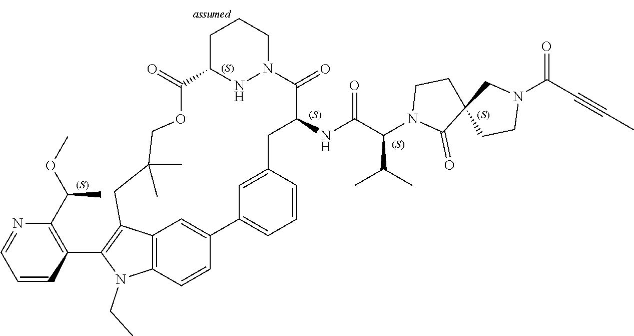 |
| A413 | 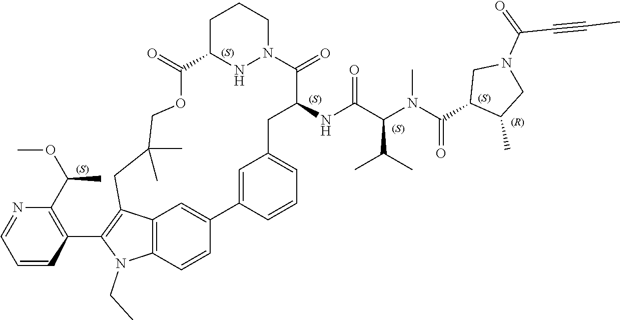 |
| A414 | 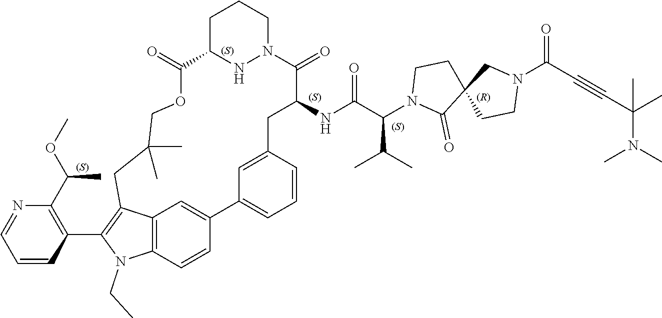 |
| A415 | 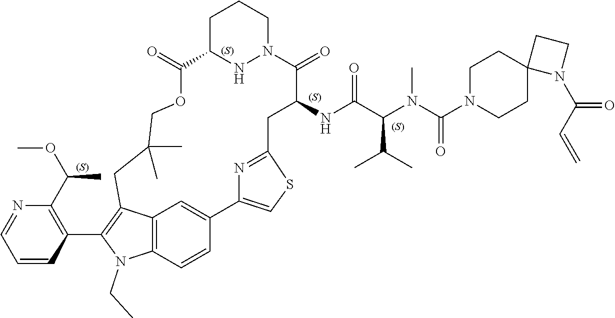 |
| A416 | 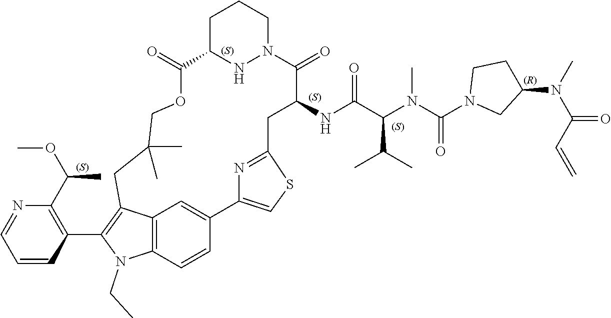 |
| A417 | 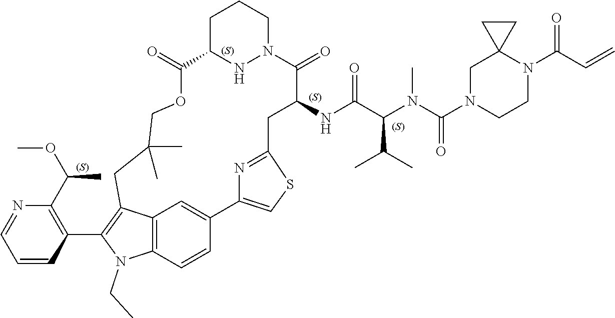 |
| A418 | 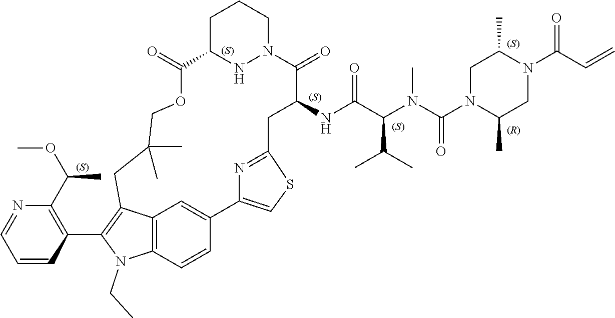 |
| A419 | 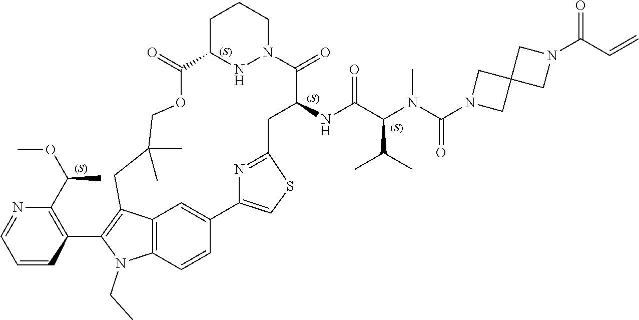 |
| A420 | 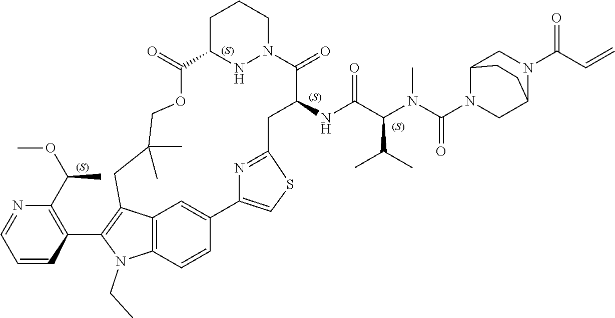 |
| A421 | 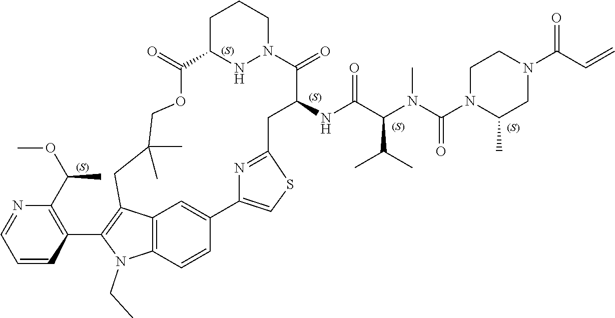 |
| A422 | 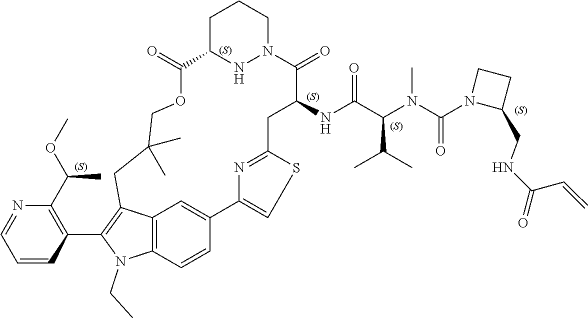 |
| A423 | 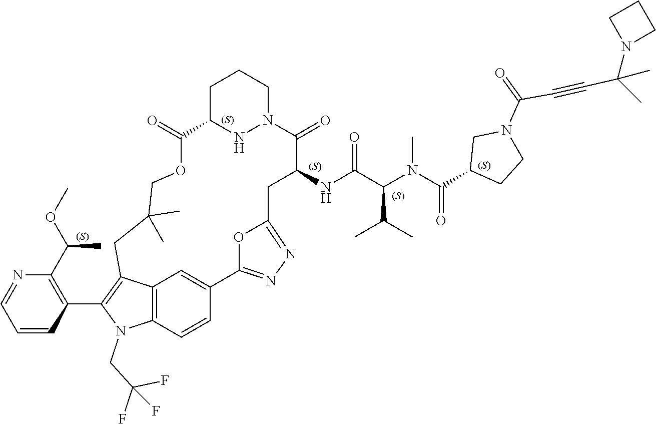 |
| A424 | 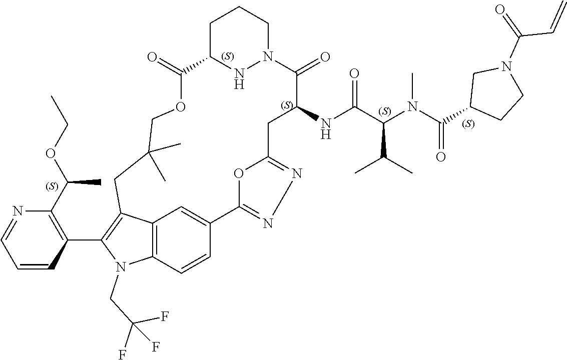 |
| A425 | 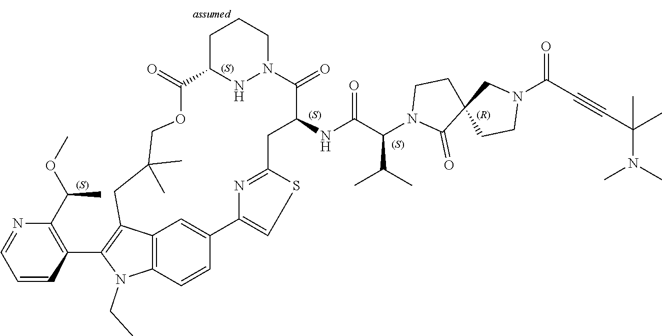 |
| A426 | 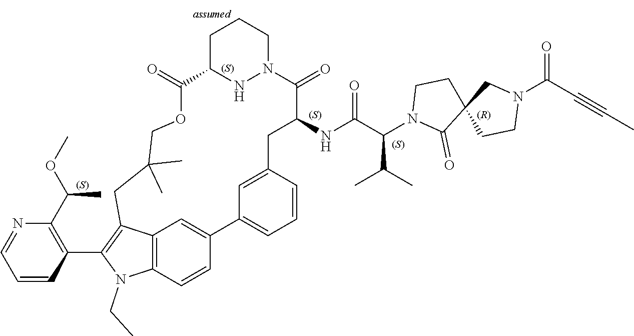 |
| A427 | 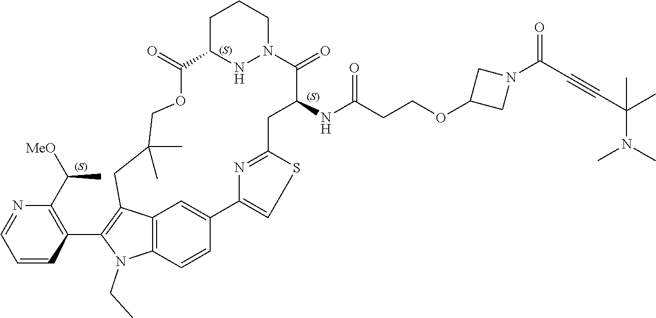 |
| A428 | 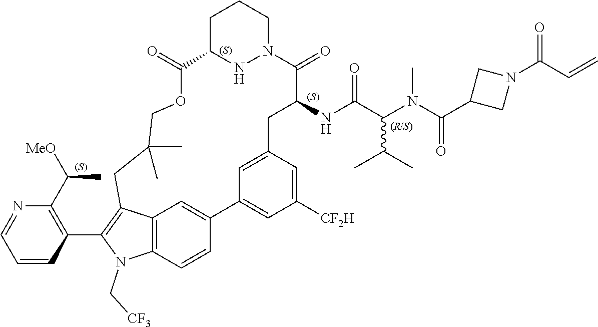 |
| A429 | 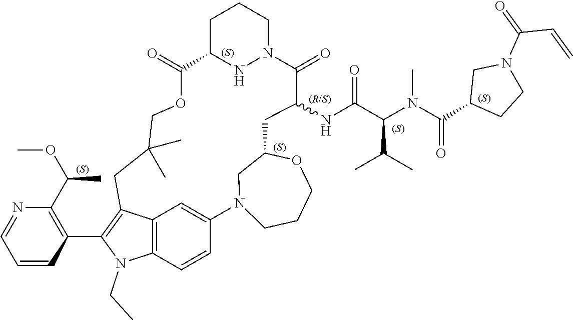 |
| A430 | 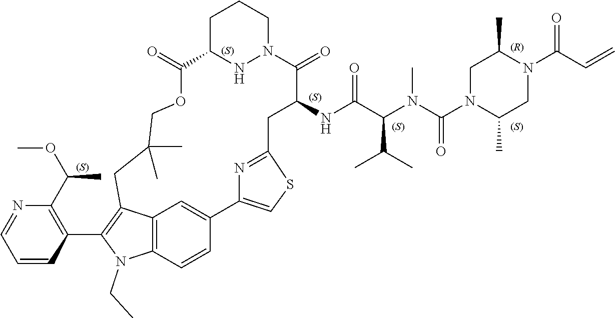 |
| A431 | 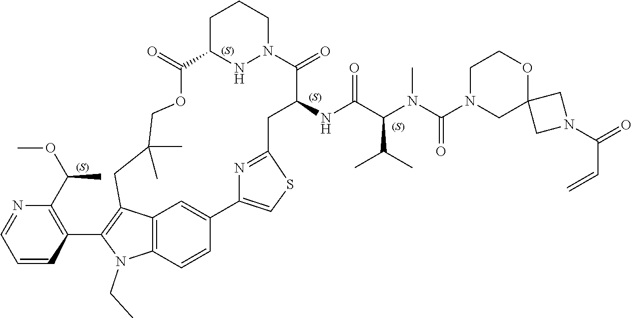 |
| A432 | 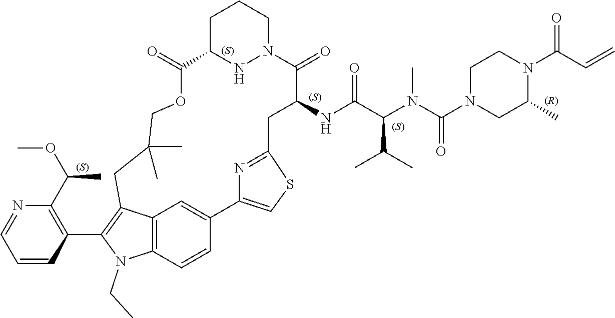 |
| A433 | 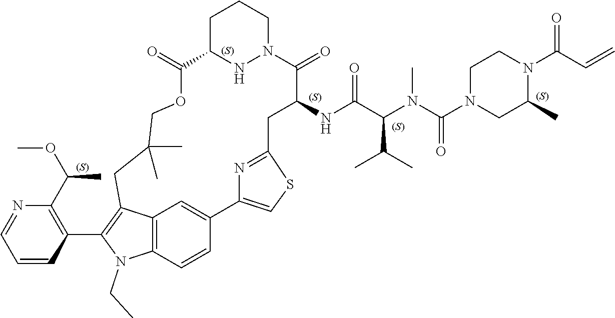 |
| A334 | 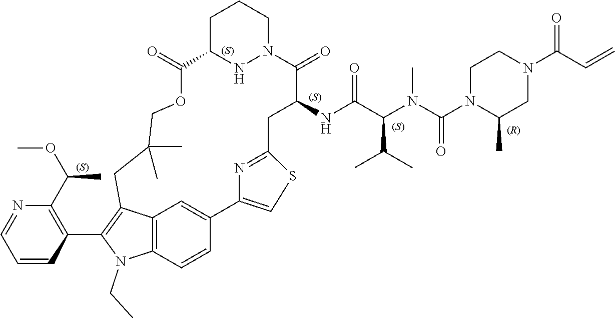 |
| A435 | 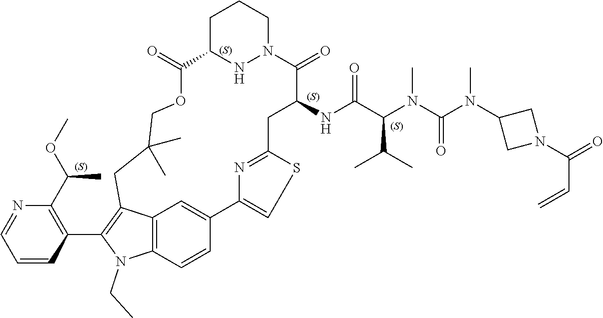 |
| A436 | 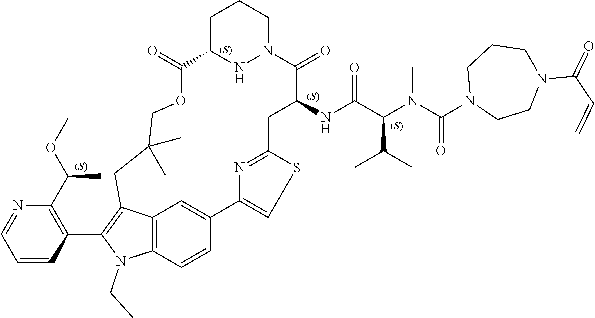 |
| A437 | 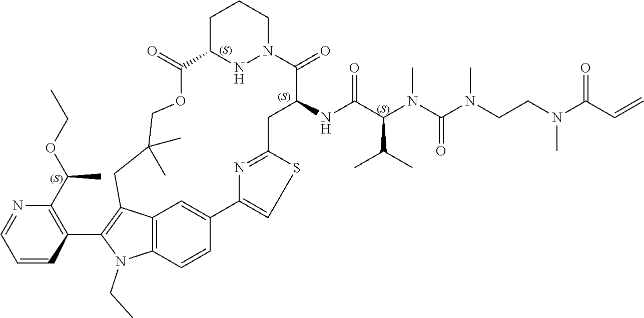 |
| A438 | 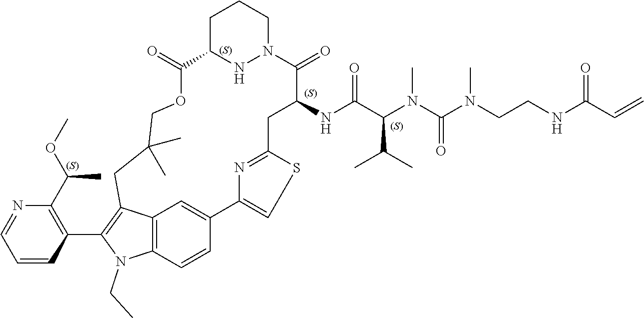 |
| A439 | 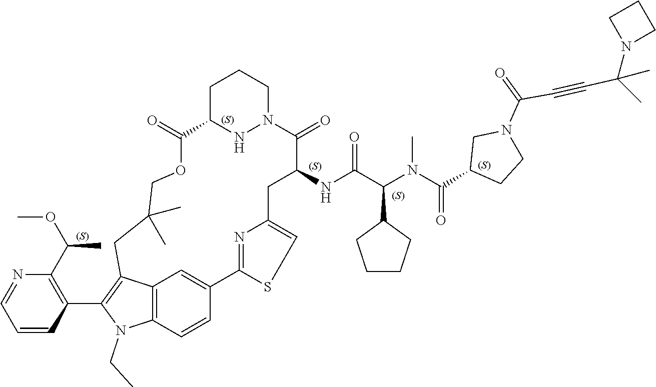 |
| A440 | 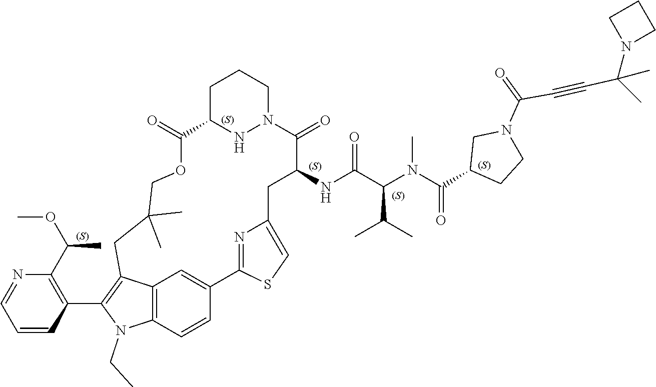 |
| A441 | 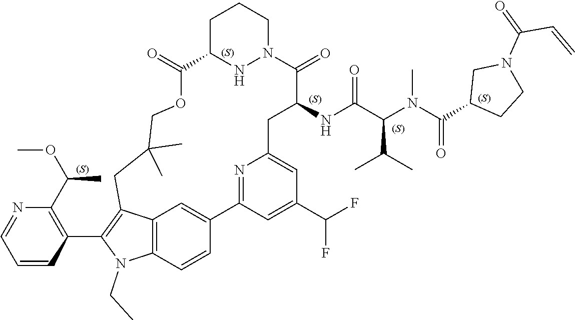 |
| A442 | 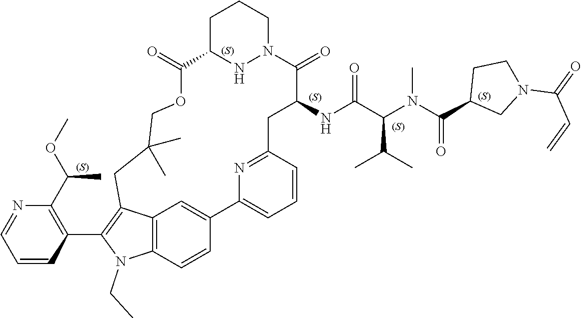 |
| A443 | 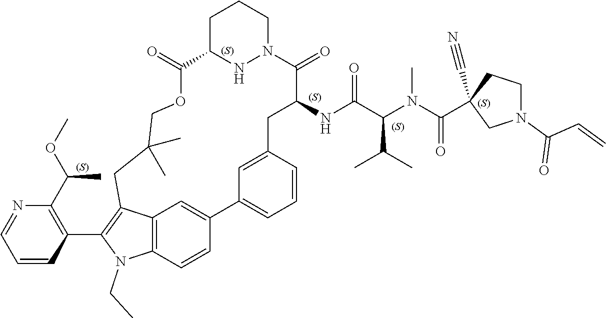 |
| A444 | 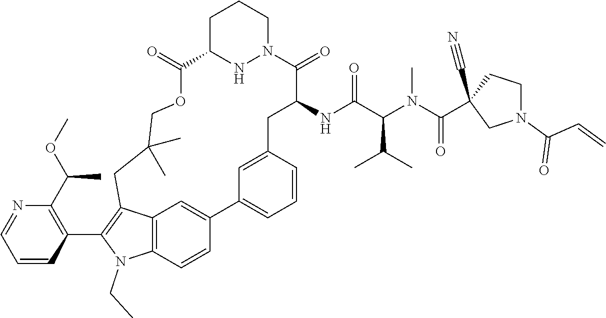 |
| A445 | 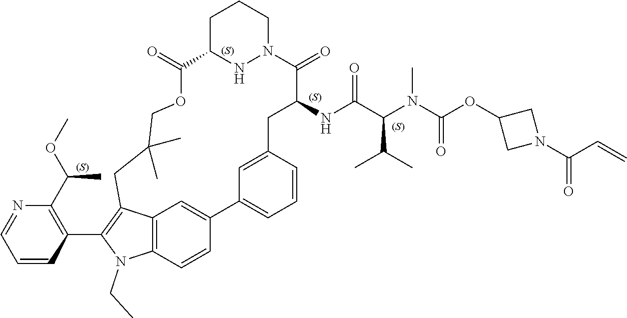 |
| A446 | 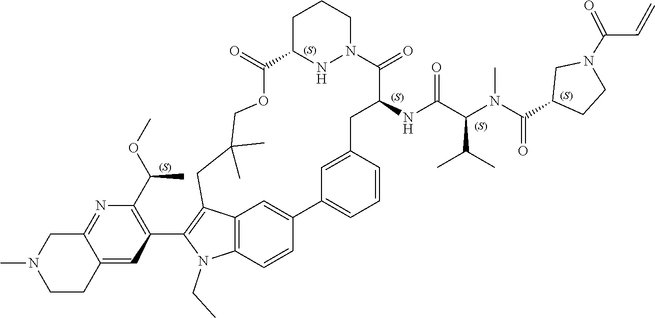 |
| A447 | 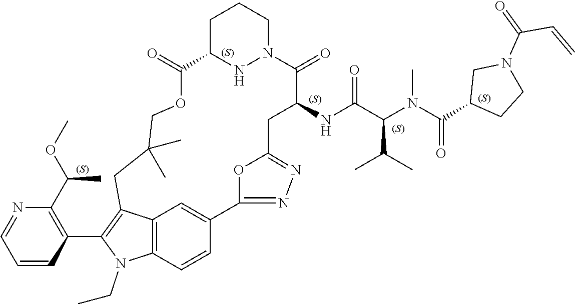 |
| A448 | 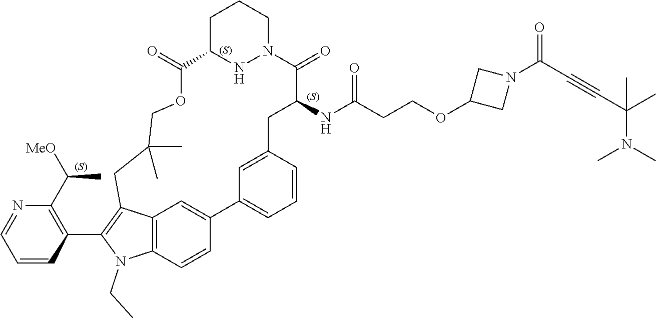 |
| A449 | 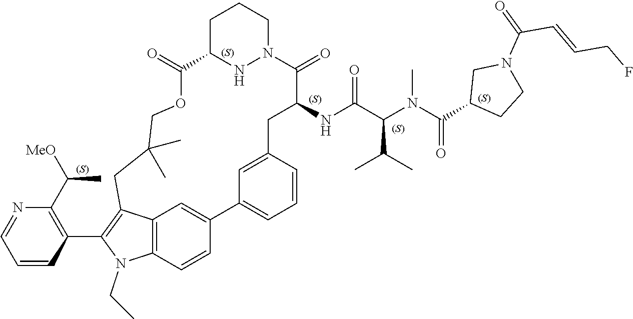 |
| A450 | 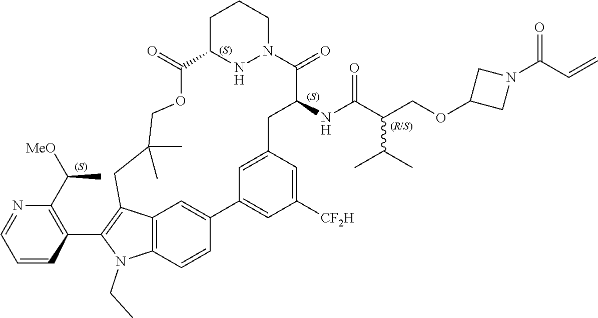 |
| A451 | 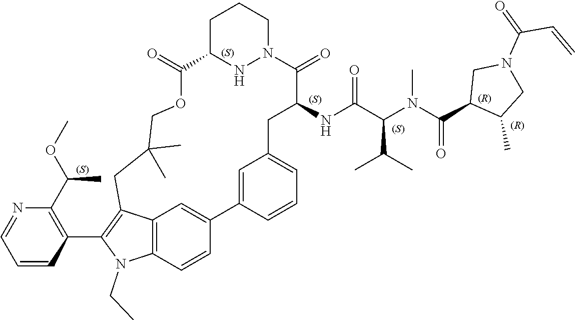 |
| A452 | 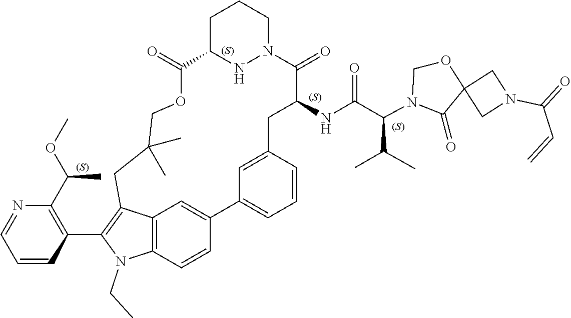 |
| A453 | 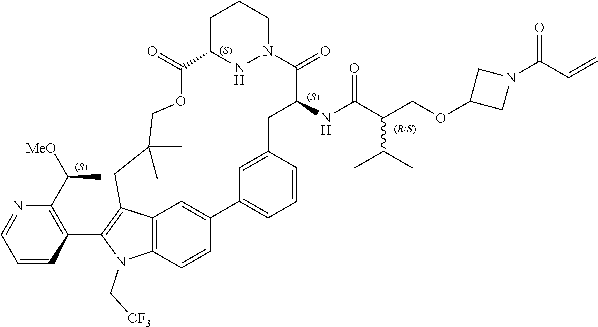 |
| A454 | 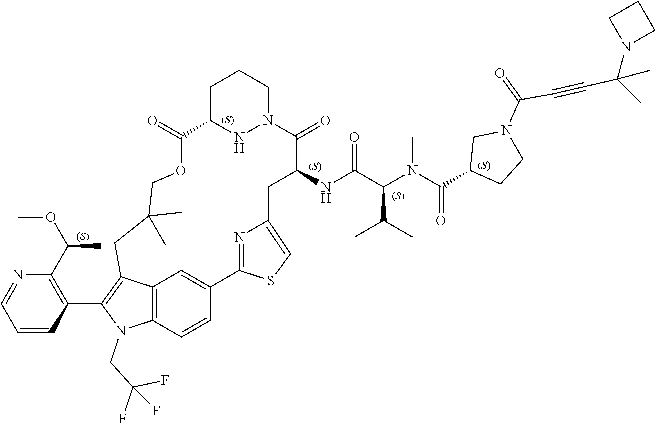 |
| A455 | 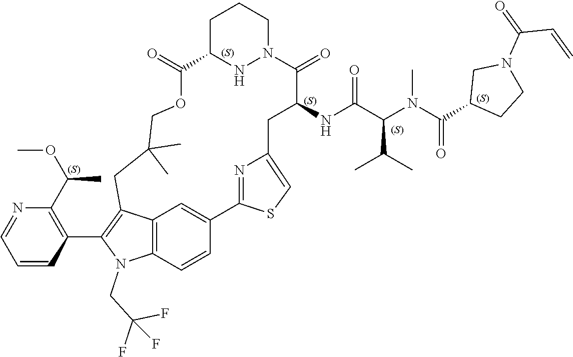 |
| A456 | 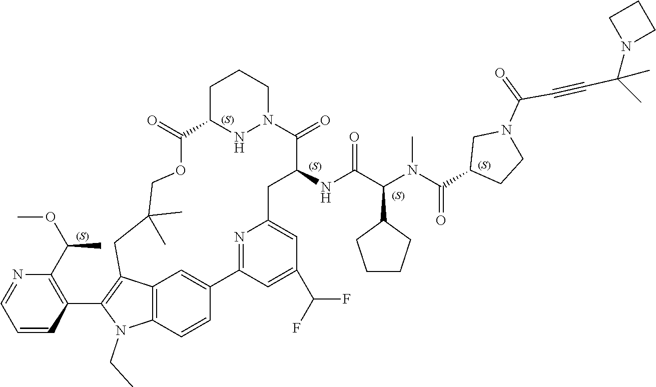 |
| A457 | 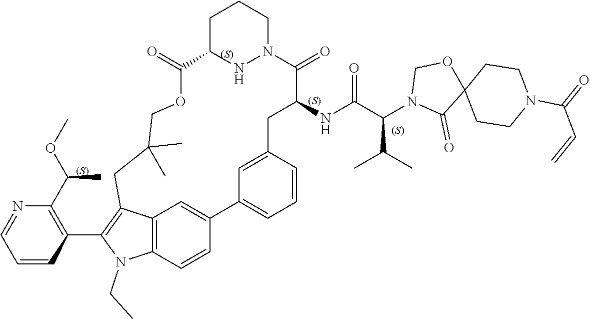 |
| A458 | 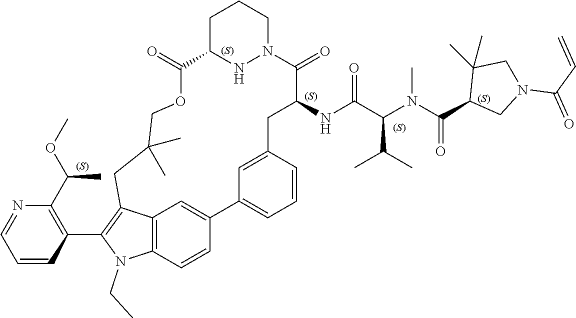 |
| A459 | 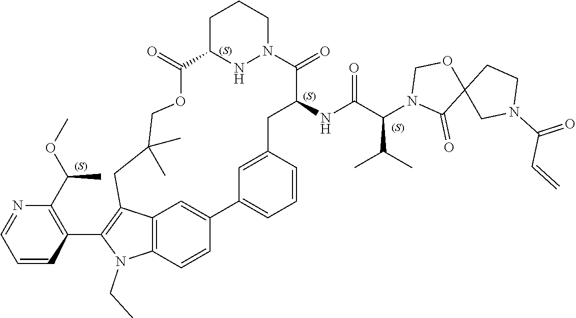 |
| A460 | 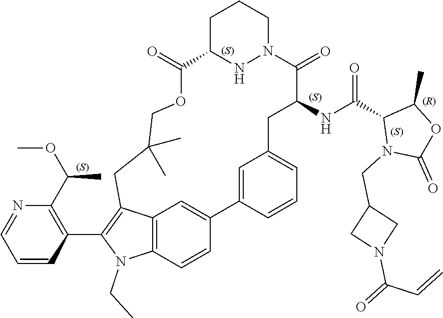 |
| A461 | 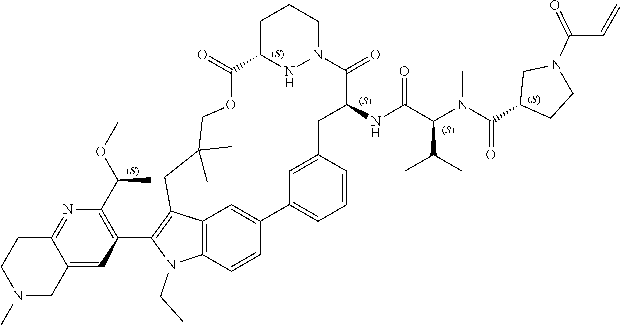 |
| A462 | 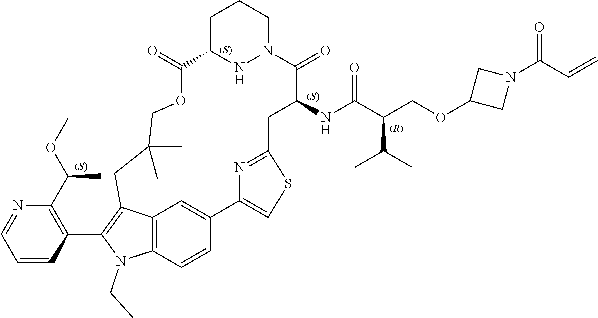 |
| A463 | 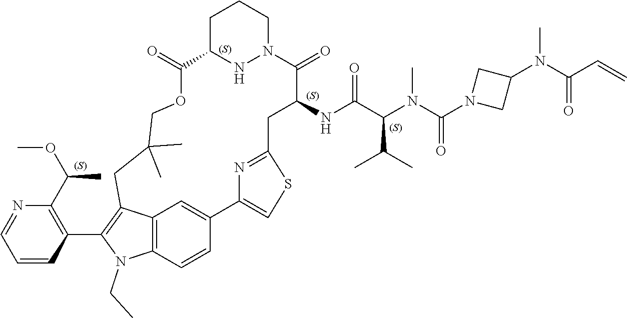 |
| A464 | 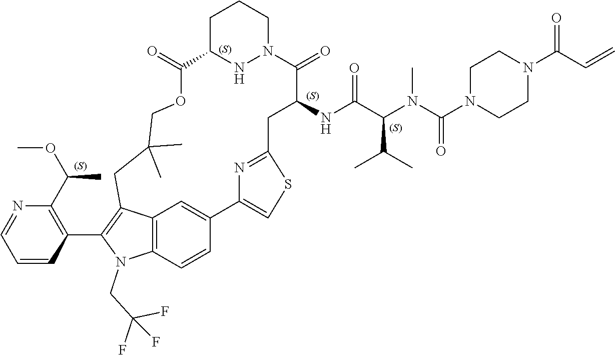 |
| A465 | 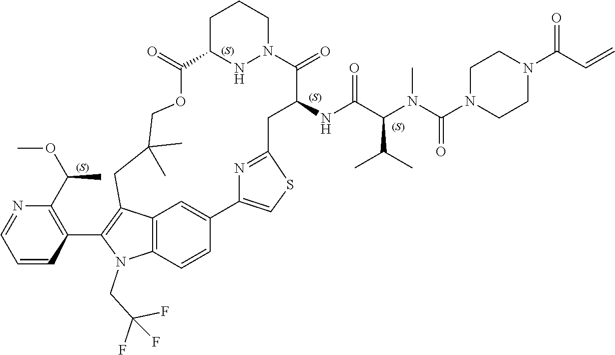 |
| A466 | 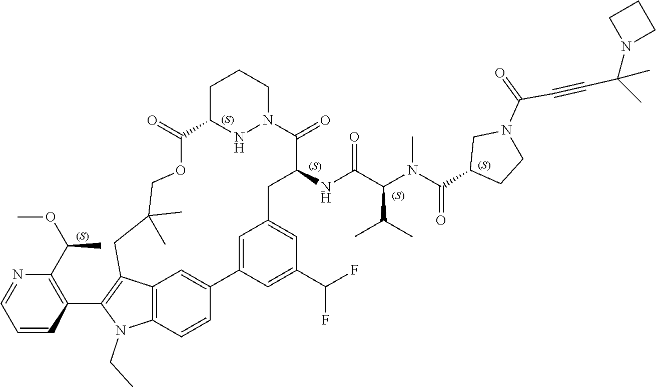 |
| A467 | 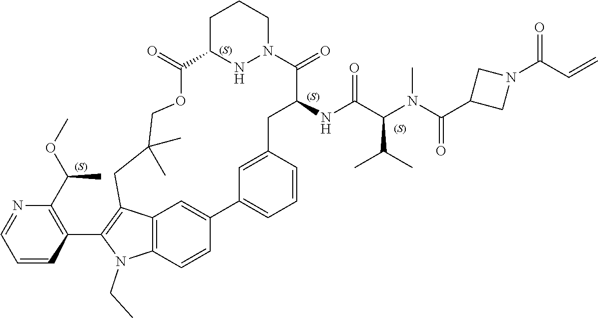 |
| A468 | 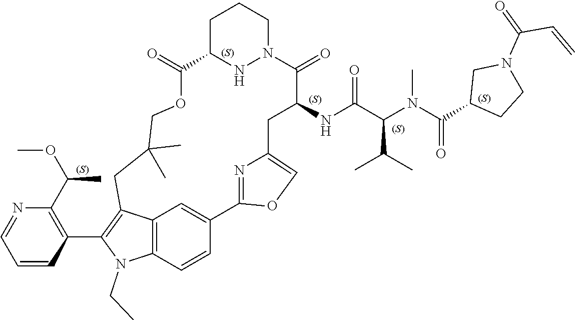 |
| A469 | 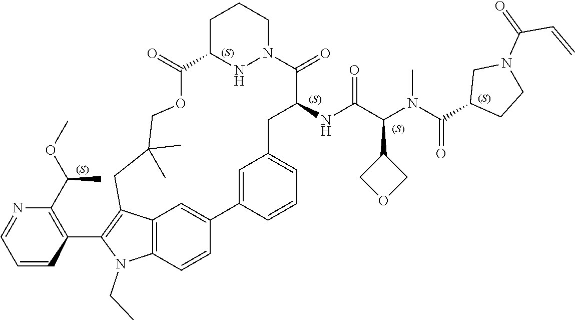 |
| A470 | 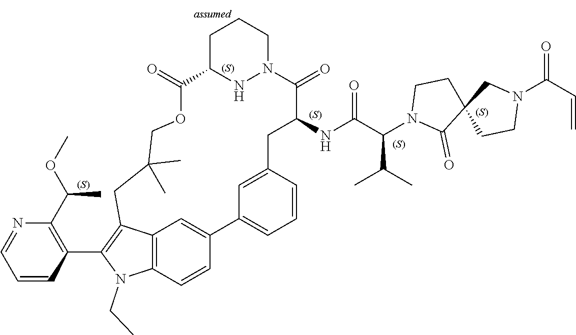 |
| A471 | 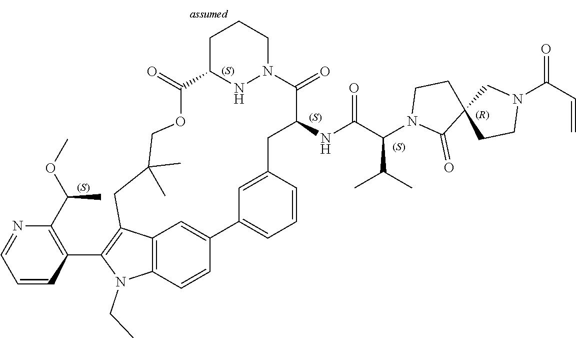 |
| A472 | 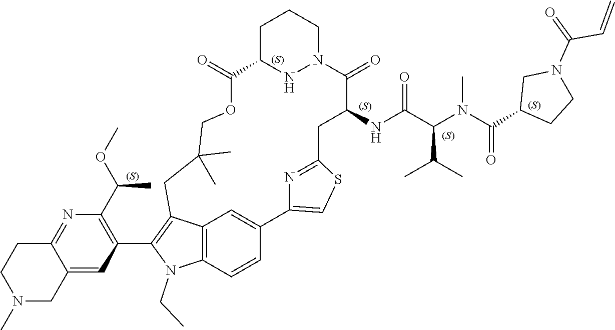 |
| A473 | 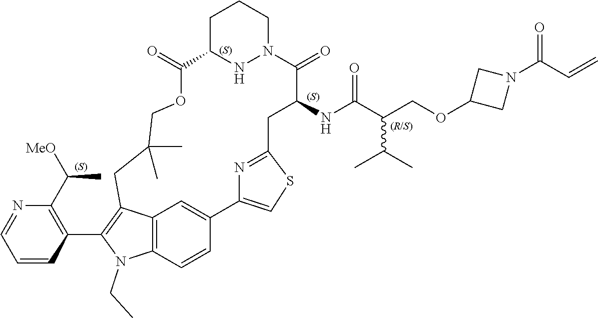 |
| A474 | 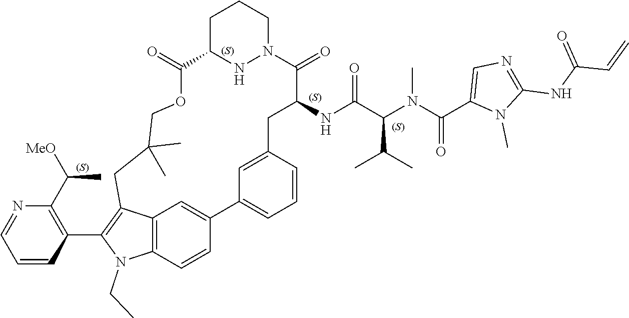 |
| A475 | 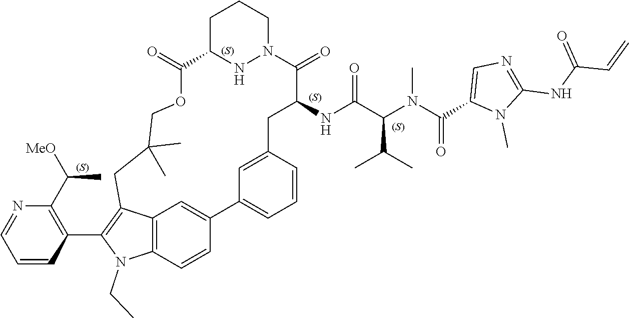 |
| A476 | 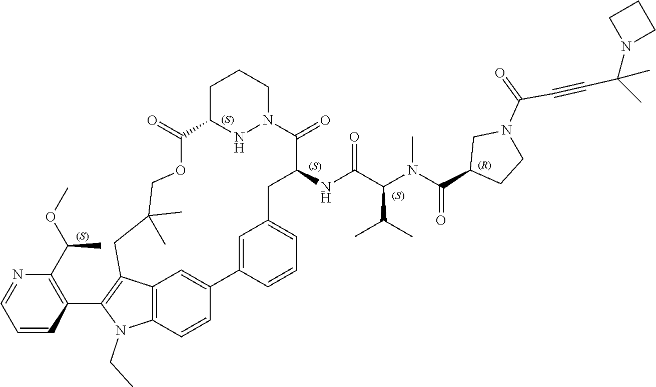 |
| A477 | 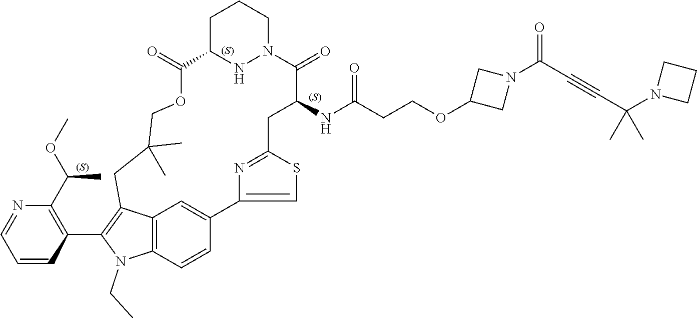 |
| A478 | 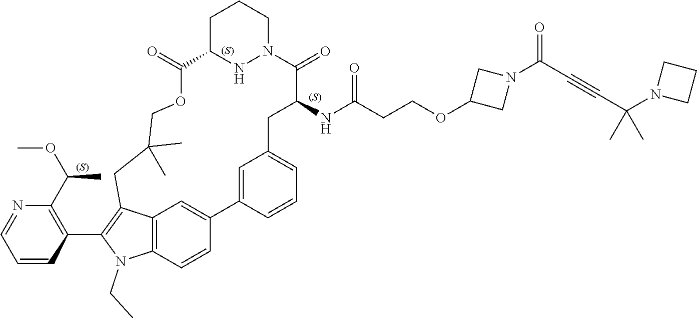 |
| A479 | 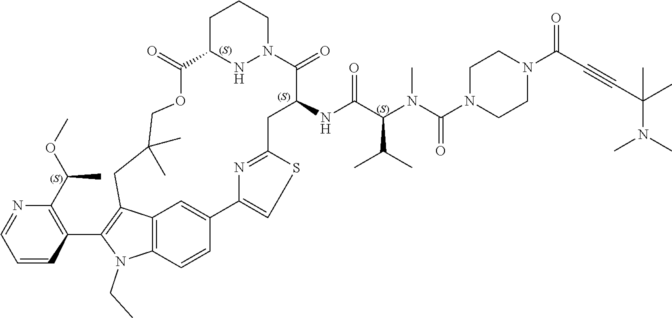 |
| A480 | 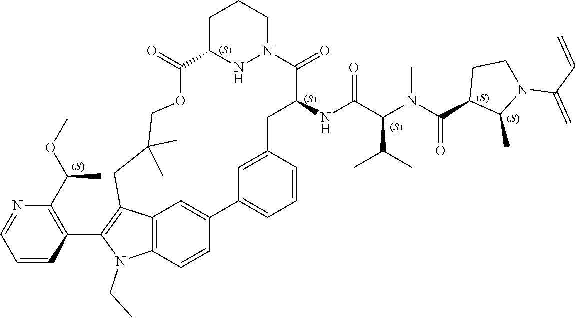 |
| A481 | 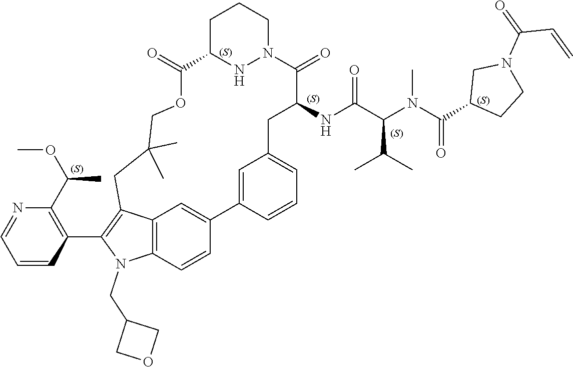 |
| A482 | 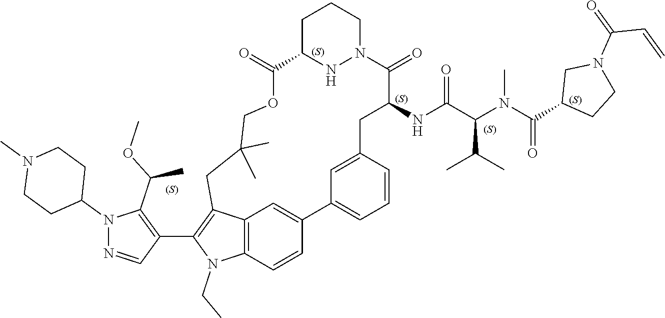 |
| A483 | 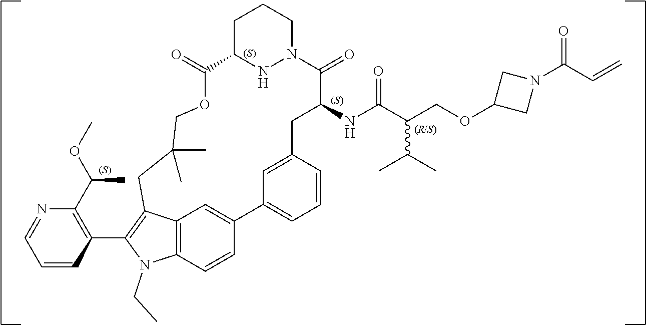 |
| A484 | 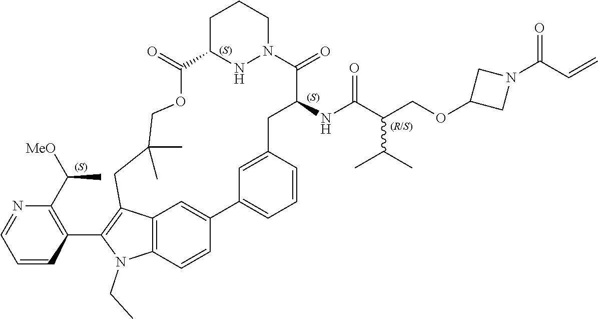 |
| A485 | 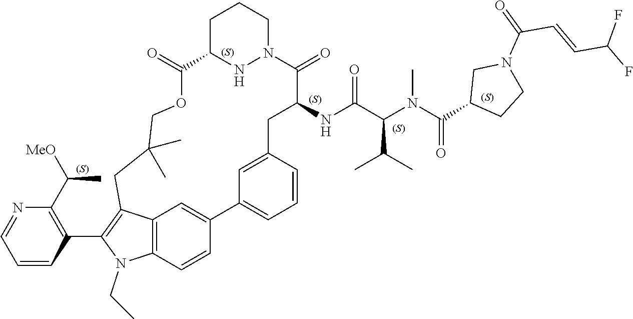 |
| A486 | 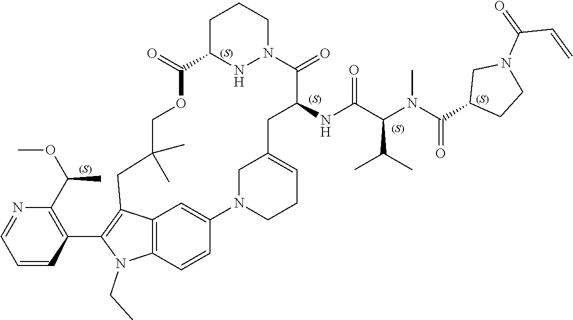 |
| A487 | 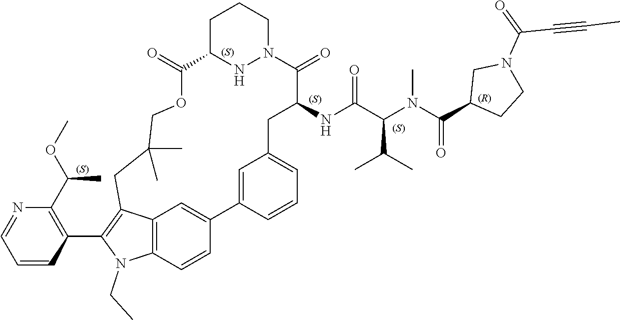 |
| A488 | 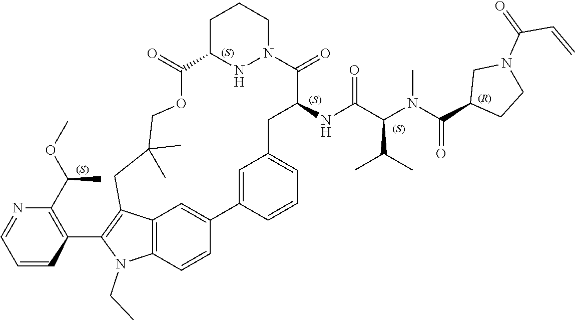 |
| A489 | 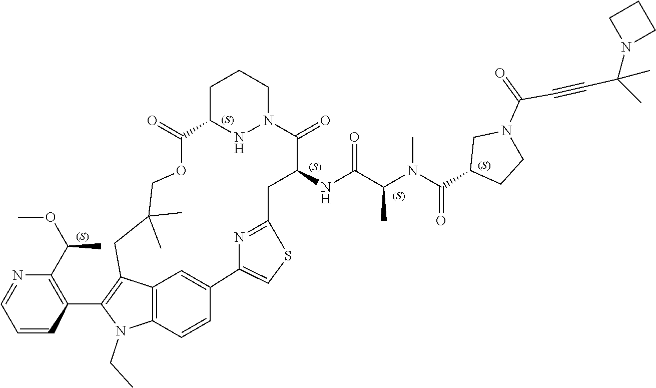 |
| A490 | 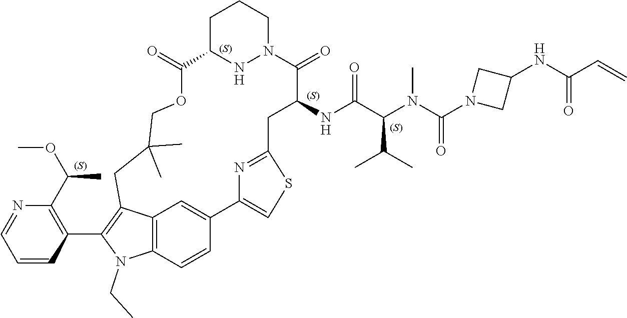 |
| A491 | 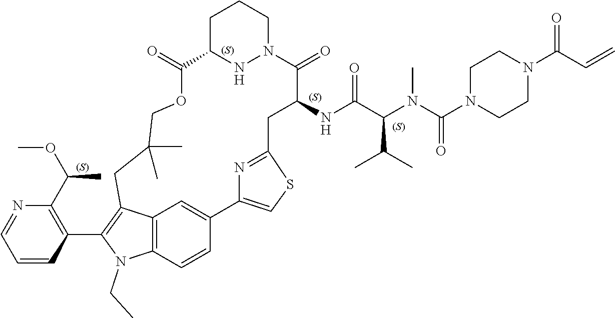 |
| A492 | 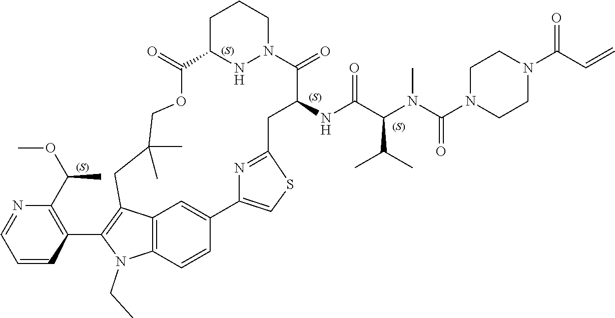 |
| A493 | 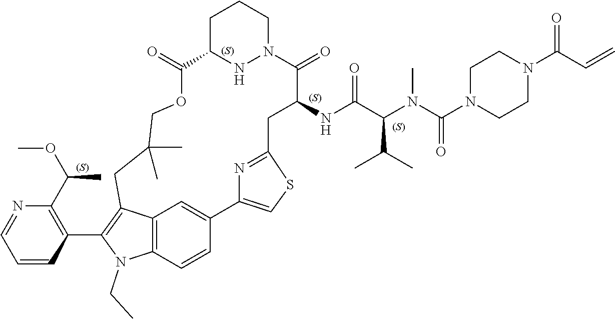 |
| A494 | 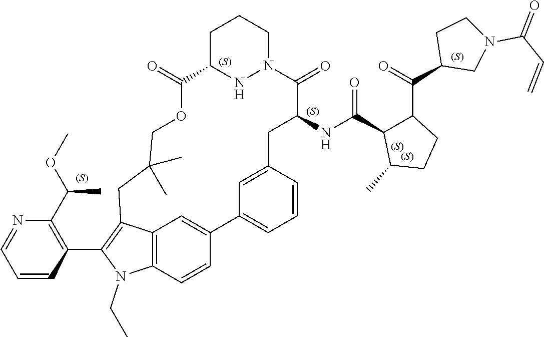 |
| A495 | 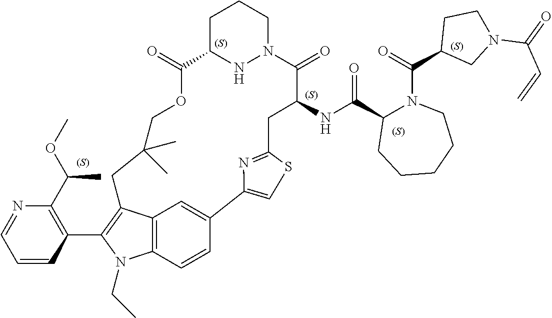 |
| A496 | 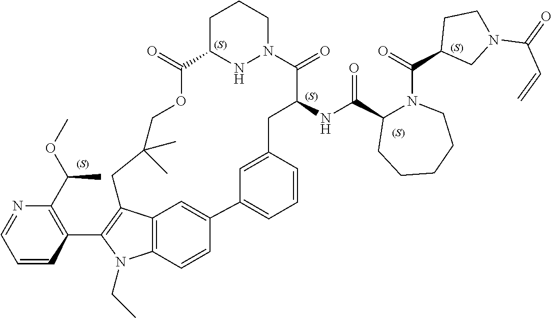 |
| A497 | 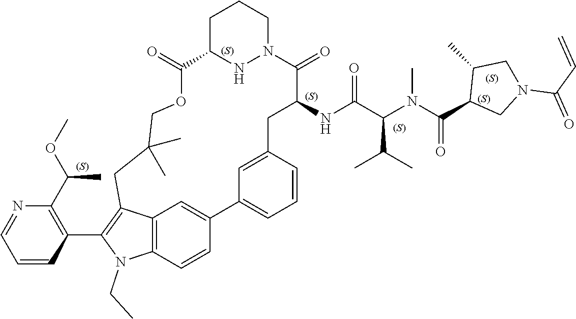 |
| A498 | 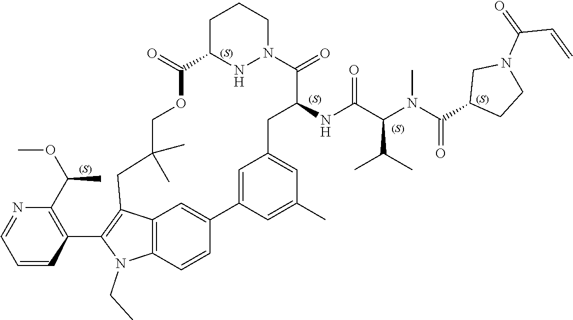 |
| A499 | 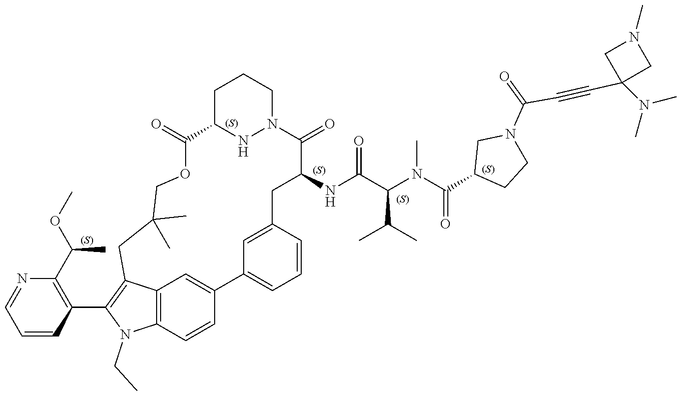 |
| A500 | 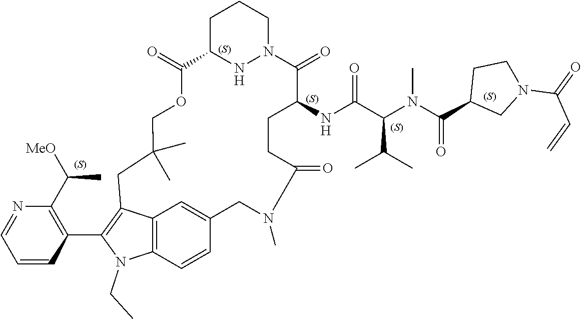 |
| A501 | 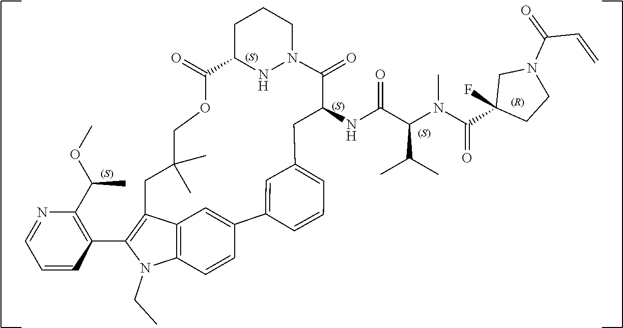 |
| A502 | 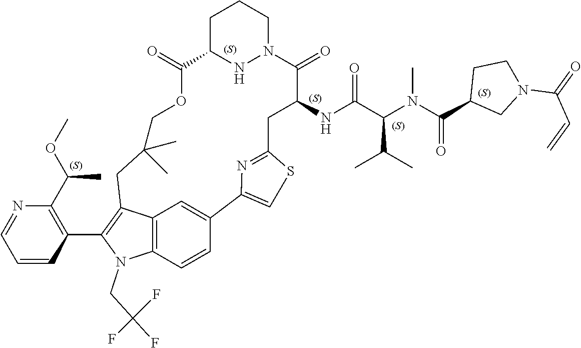 |
| A503 | 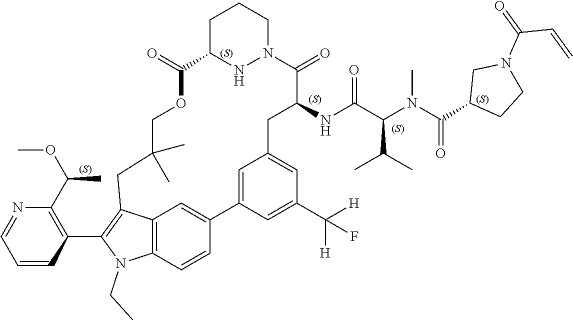 |
| A504 | 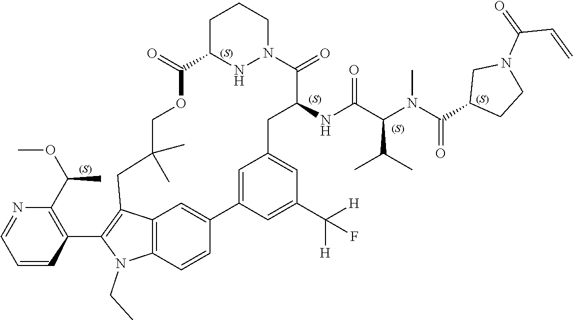 |
| A505 | 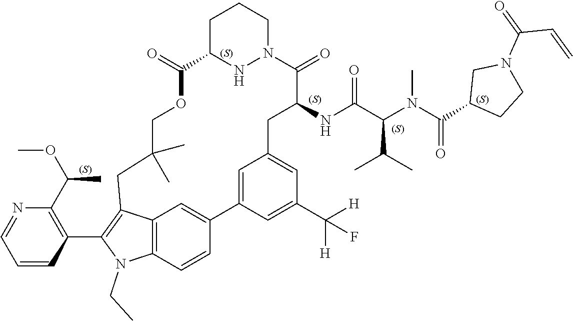 |
| A506 | 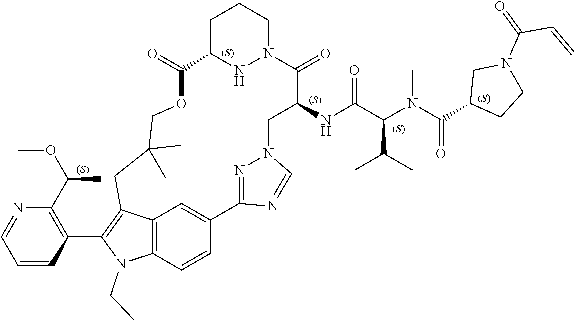 |
| A507 | 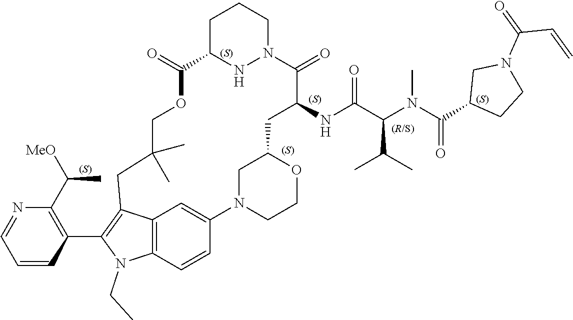 |
| A508 | 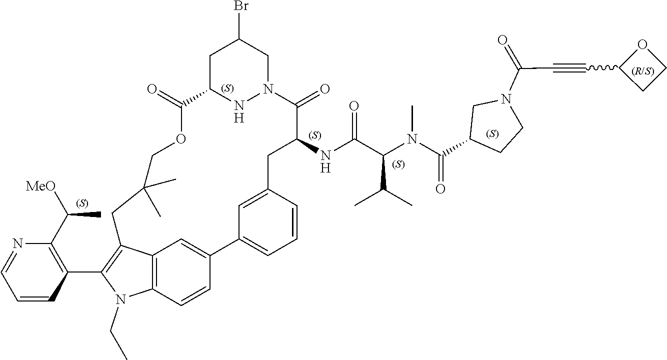 |
| A509 | 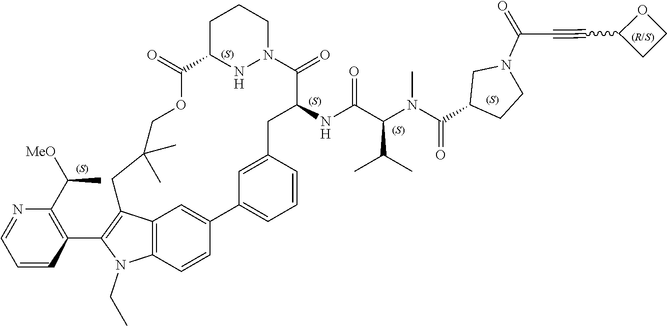 |
| A510 | 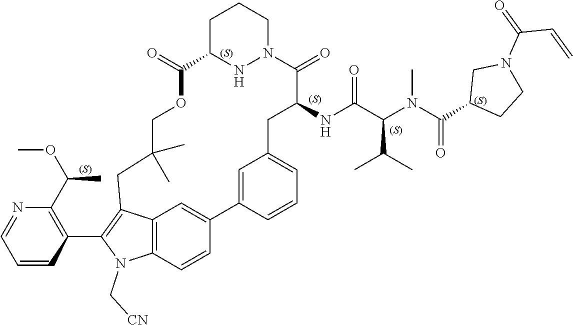 |
| A511 | 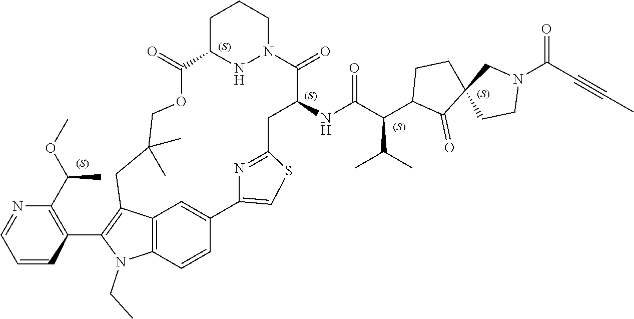 |
| A512 | 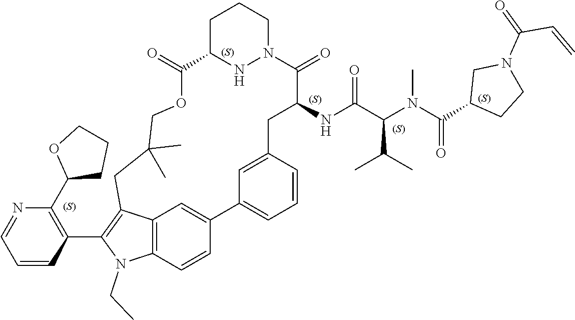 |
| A513 | 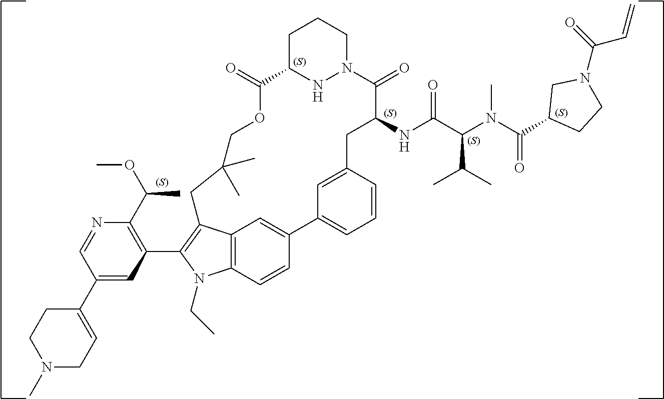 |
| A514 | 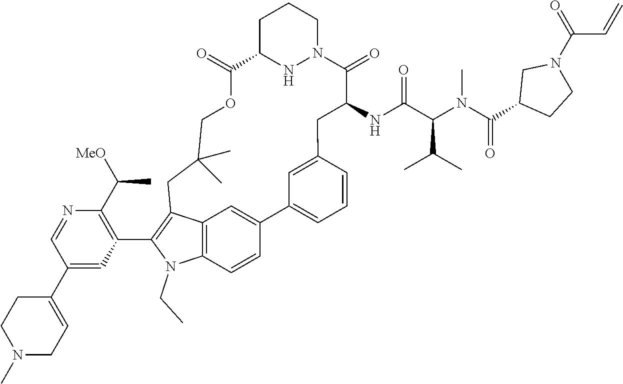 |
| A515 | 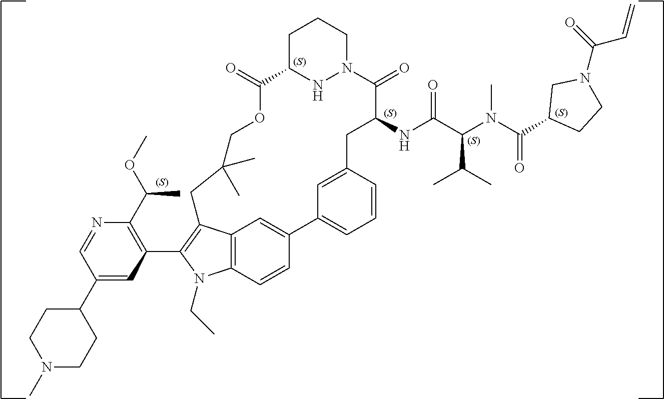 |
| A516 | 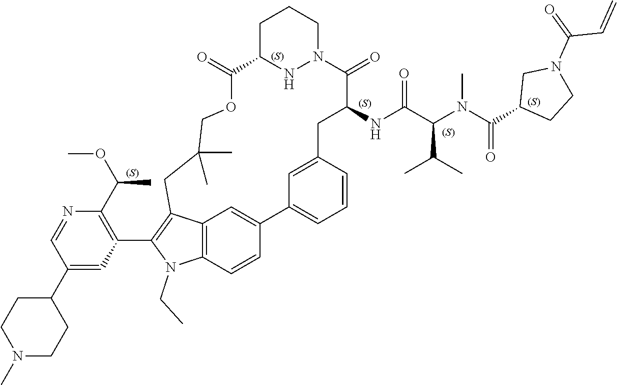 |
| A517 | 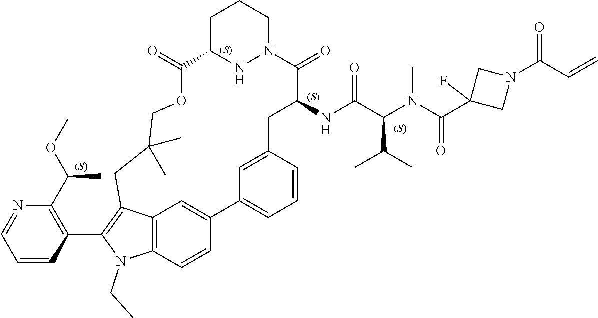 |
| A518 | 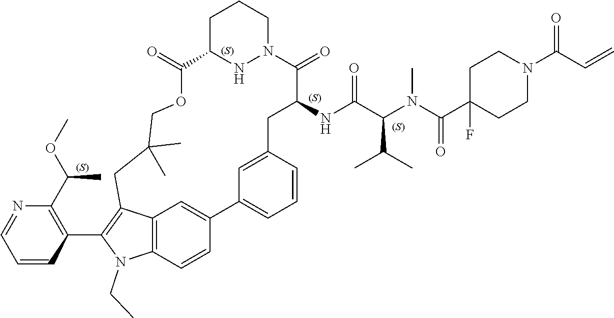 |
| A519 | 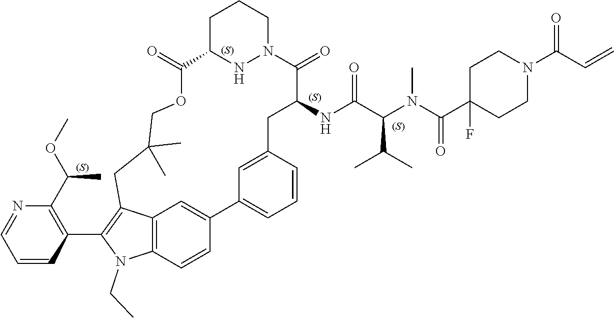 |
| A520 | 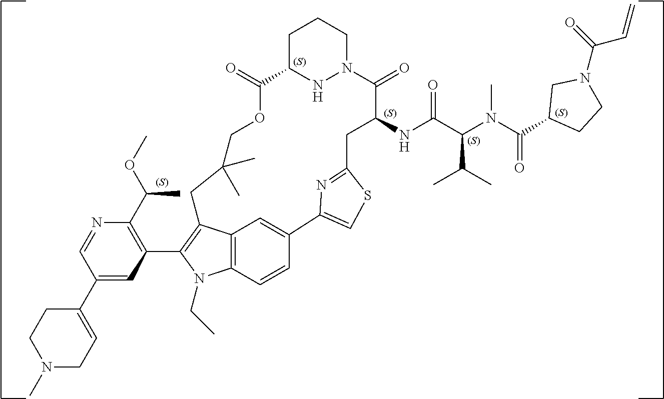 |
| A521 | 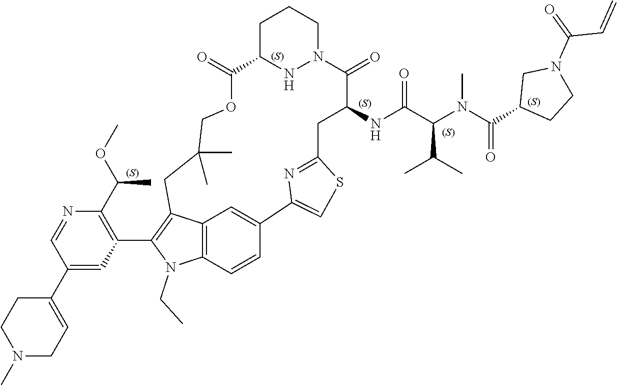 |
| A522 | 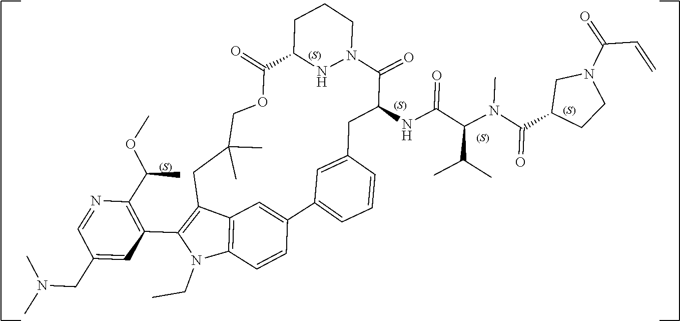 |
| A523 | 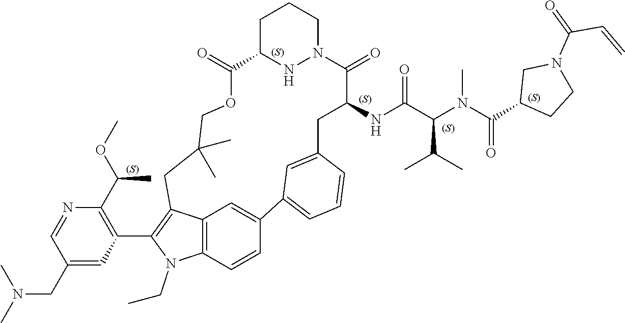 |
| A524 | 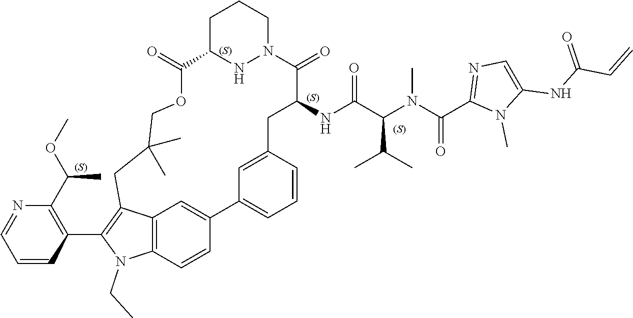 |
| A525 | 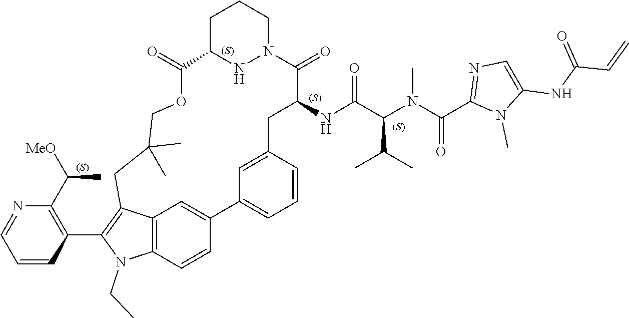 |
| A526 | 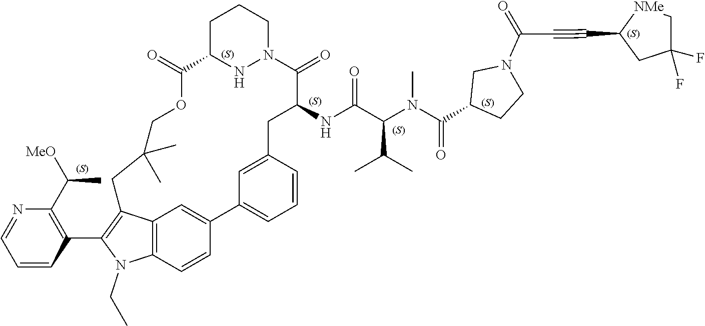 |
| A527 | 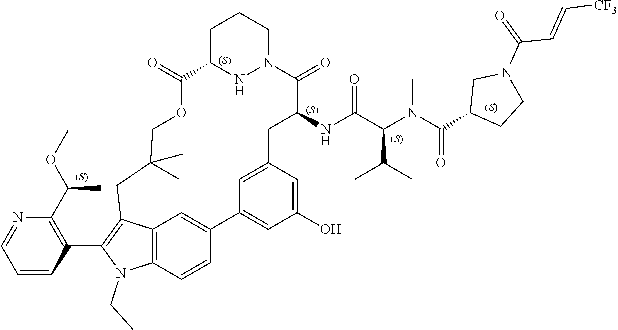 |
| A528 | 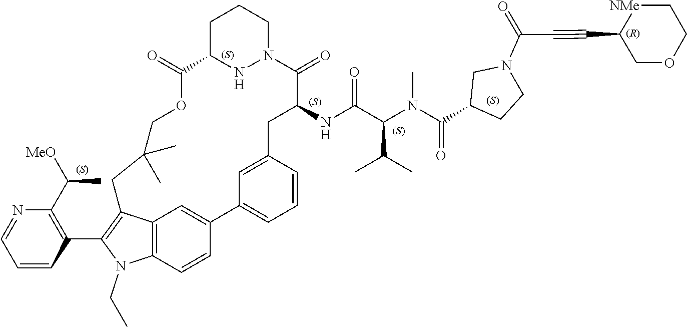 |
| A529 | 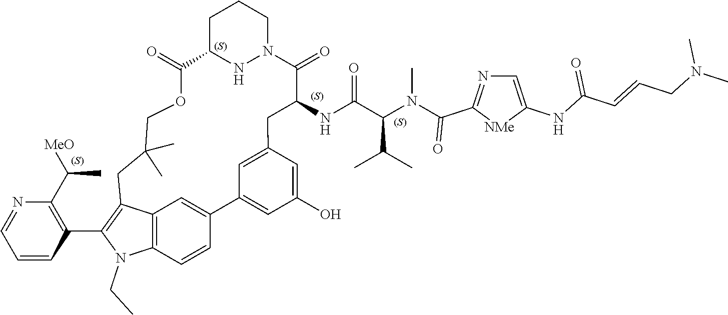 |
| A530 | 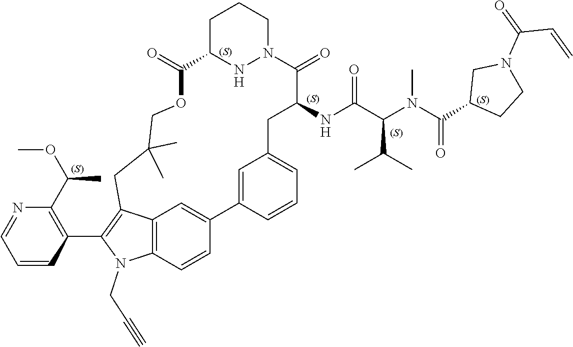 |
| A531 | 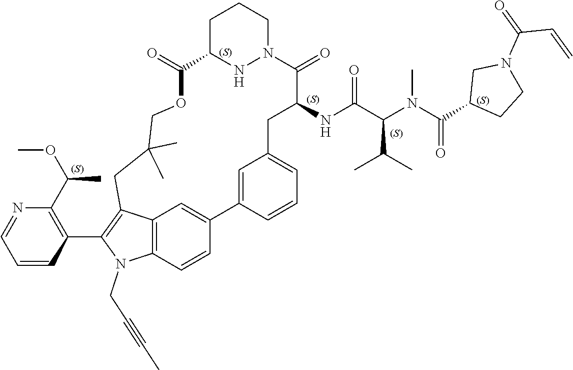 |
| A532 | 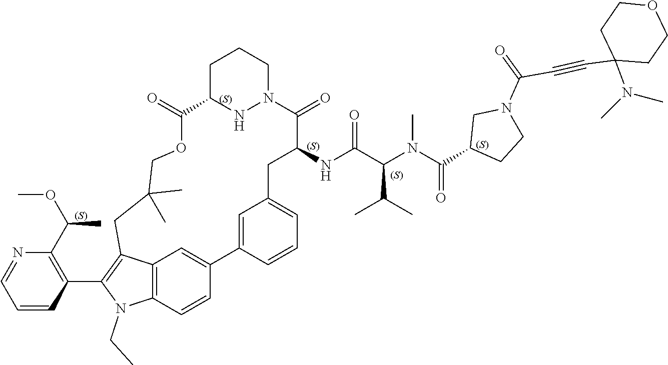 |
| A533 | 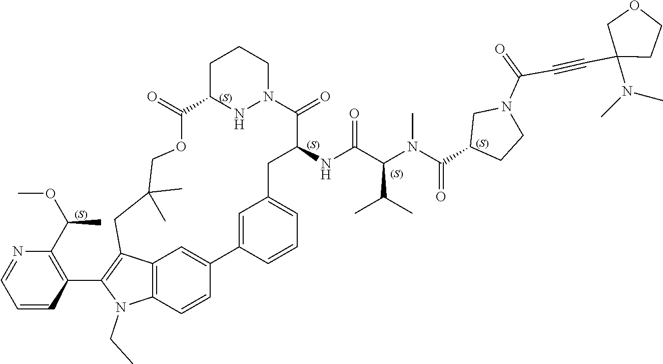 |
| A534 | 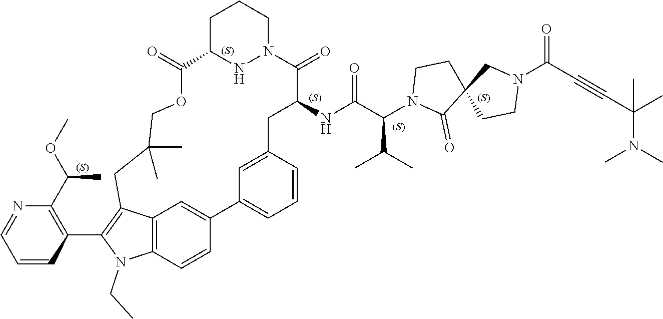 |
| A535 | 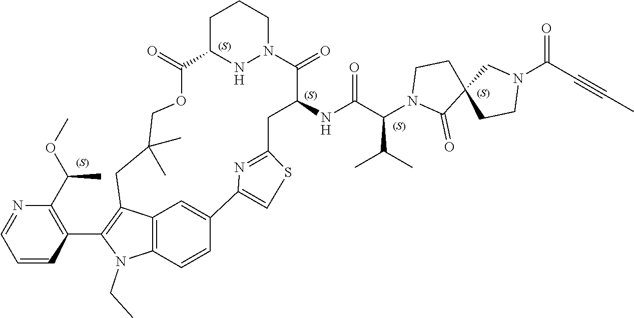 |
| A536 | 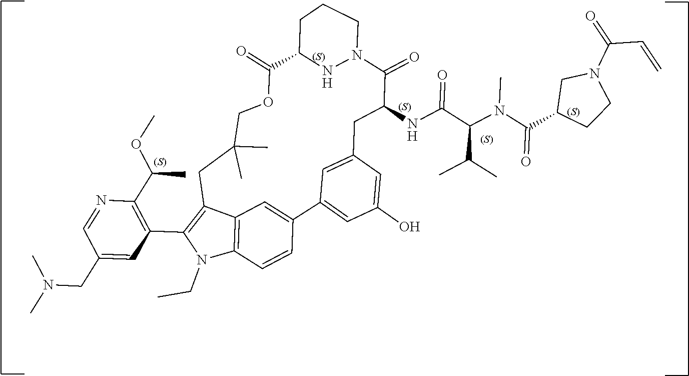 |
| A537 | 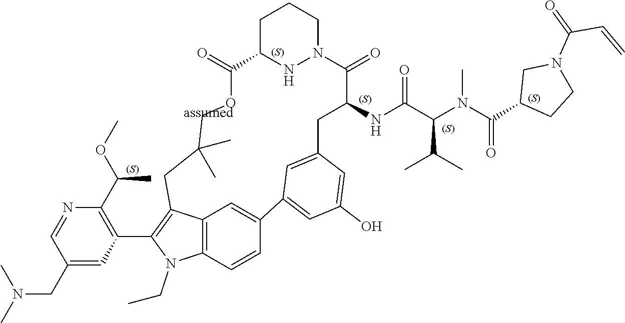 |
| A538 | 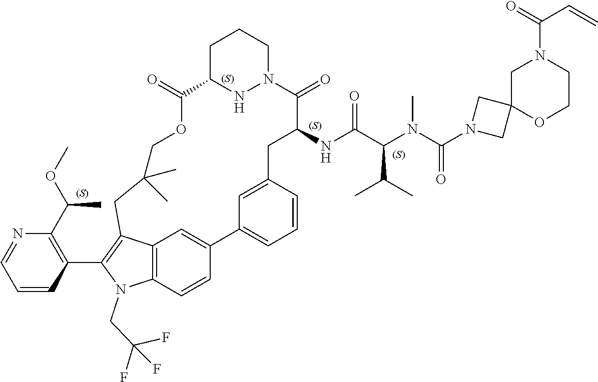 |
| A539 | 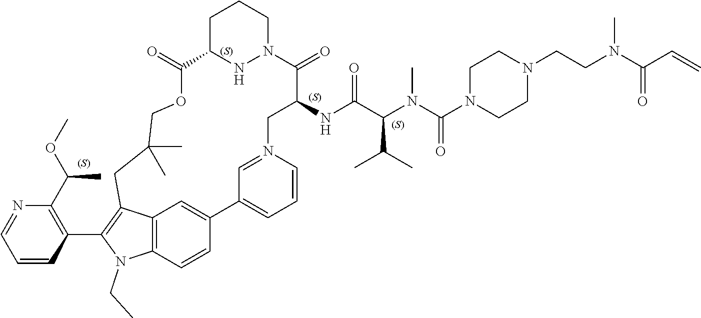 |
| A540 | 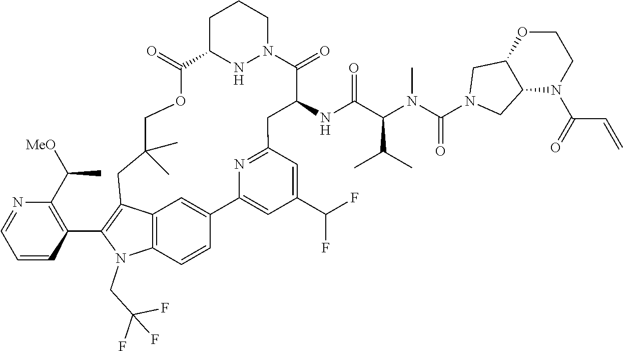 |
| A541 | 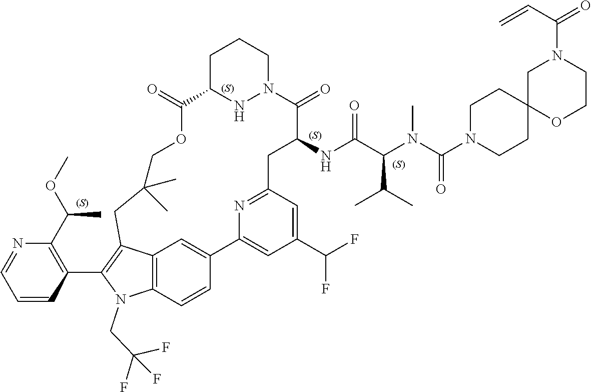 |
| A542 | 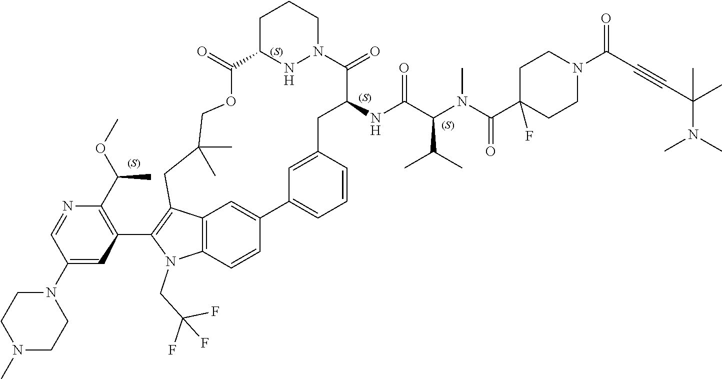 |
| A543 | 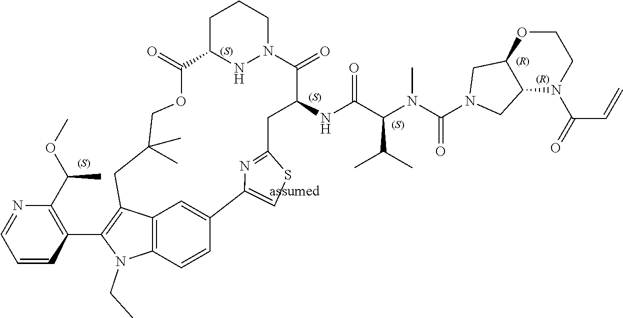 |
| A544 | 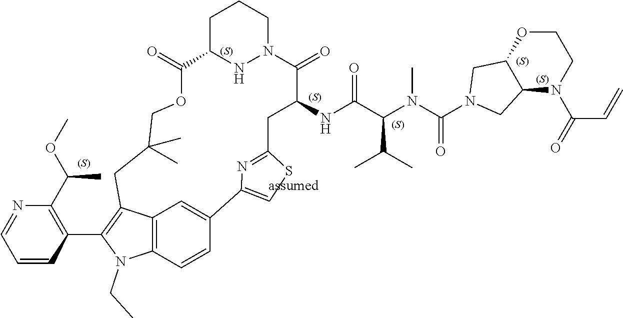 |
| A545 | 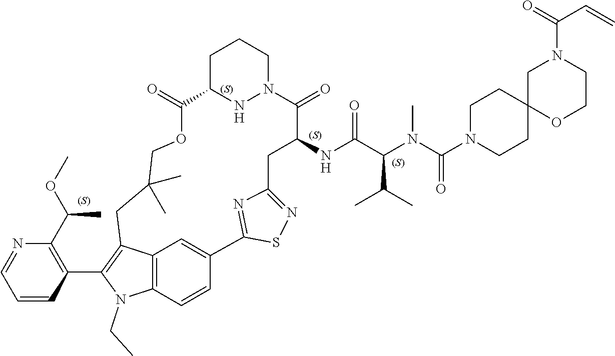 |
| A546 | 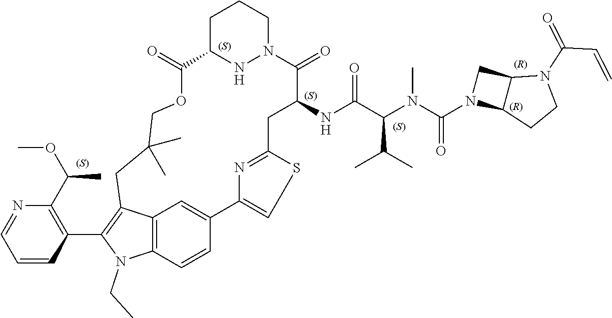 |
| A547 | 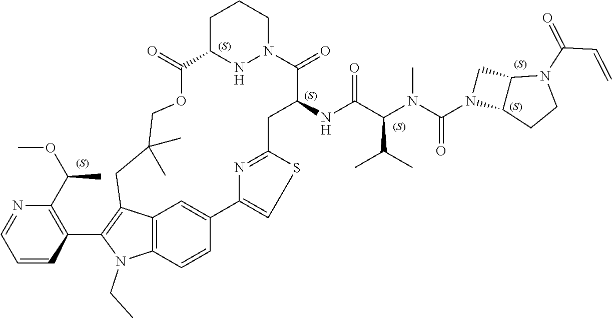 |
| A548 | 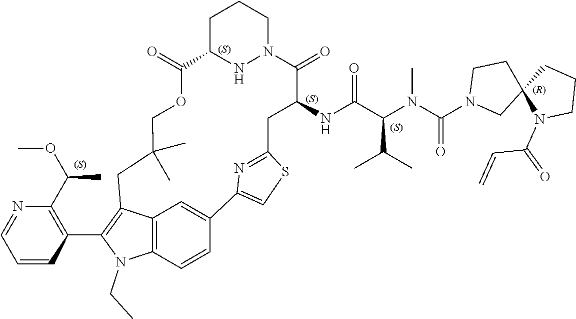 |
| A549 | 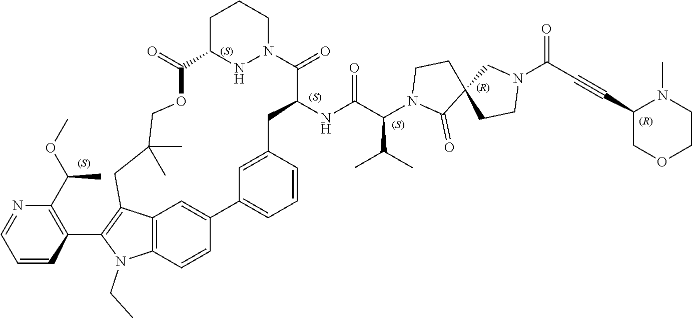 |
| A550 | 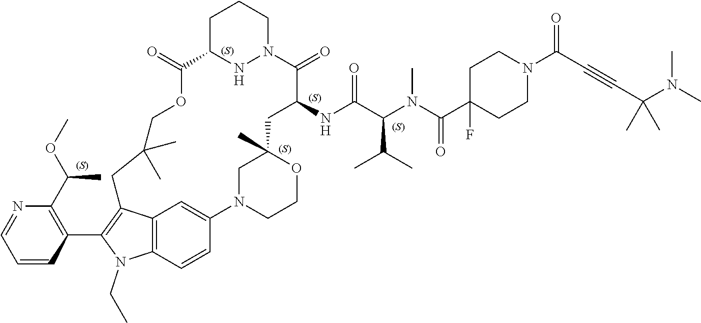 |
| A551 | 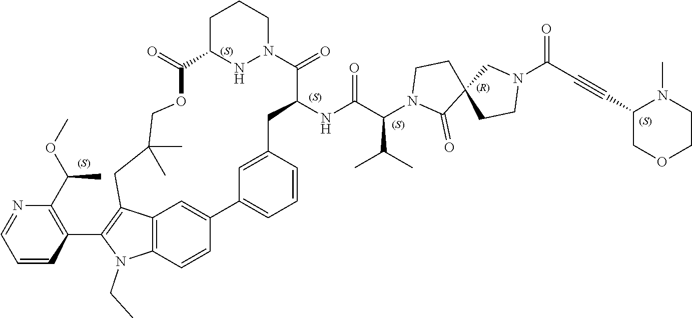 |
| A552 | 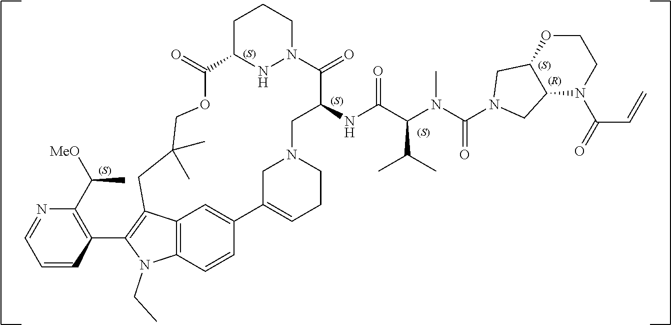 |
| A553 | 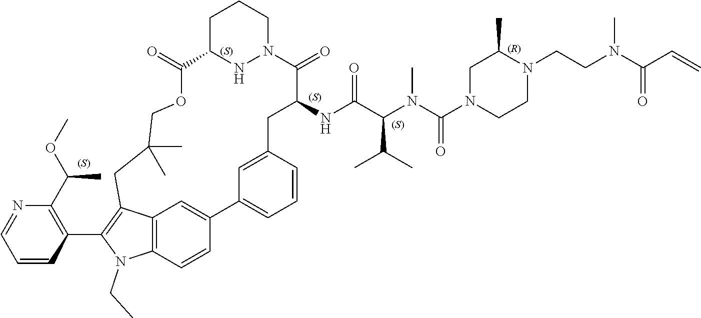 |
| A554 | 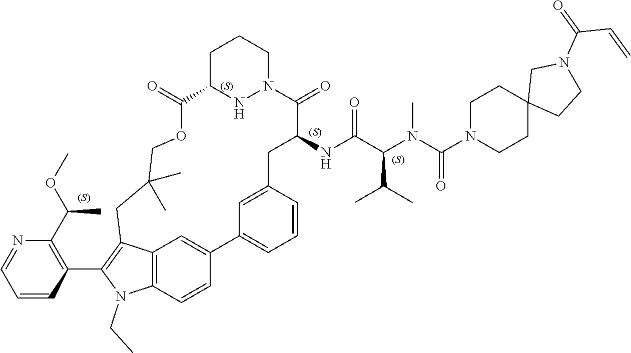 |
| A555 | 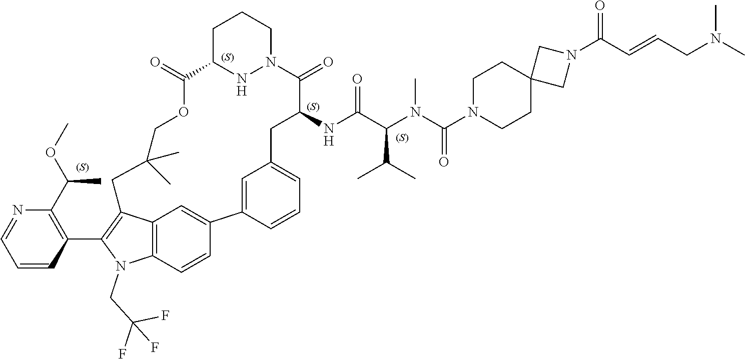 |
| A556 | 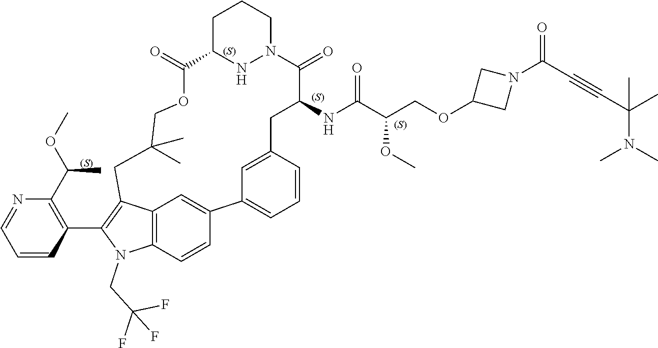 |
| A557 | 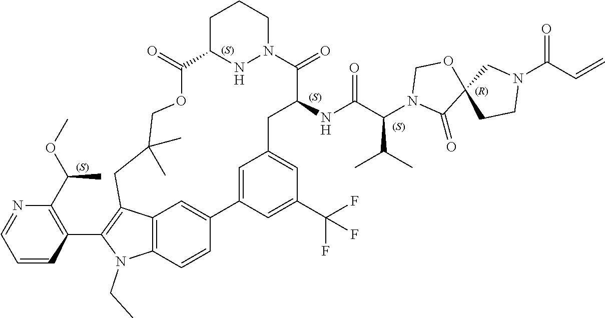 |
| A558 | 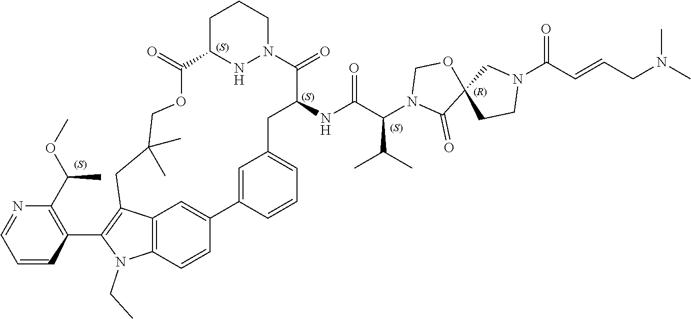 |
| A559 | 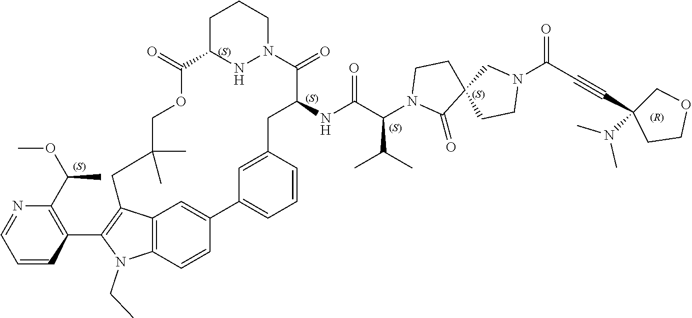 |
| A560 | 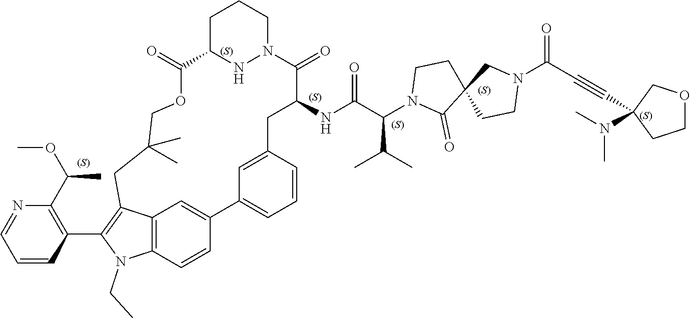 |
| A561 | 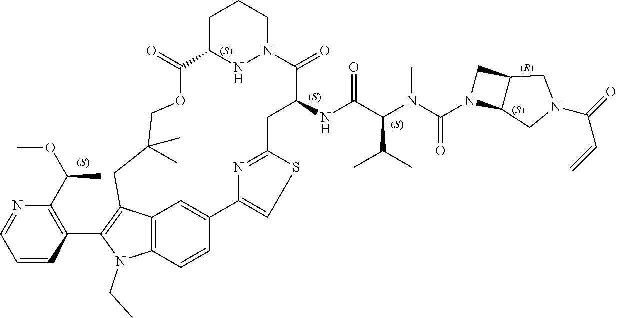 |
| A562 | 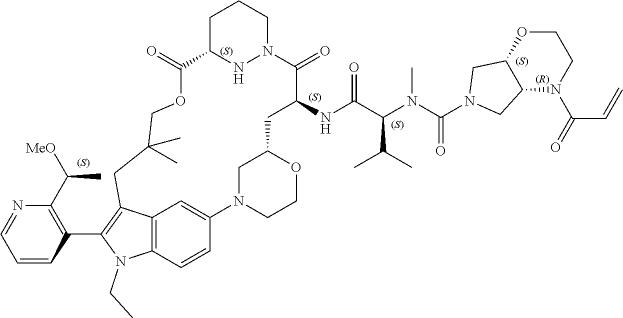 |
| A563 | 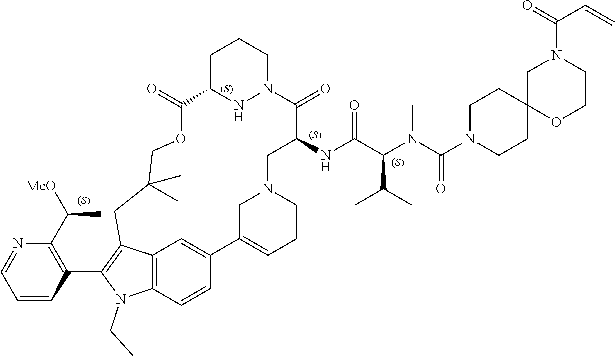 |
| A564 | 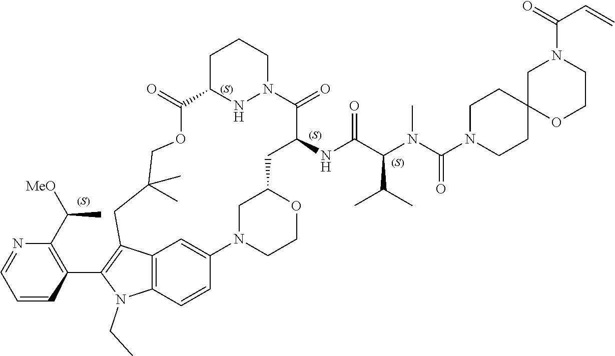 |
| A565 | 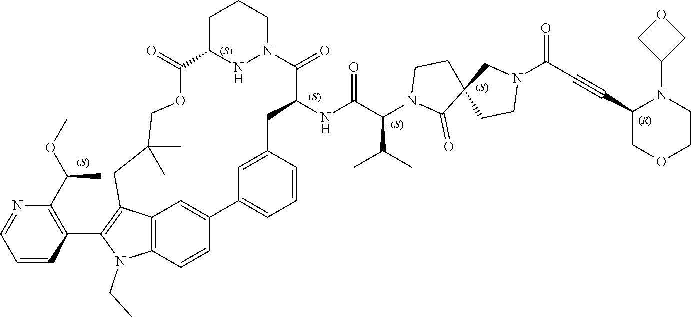 |
| A566 | 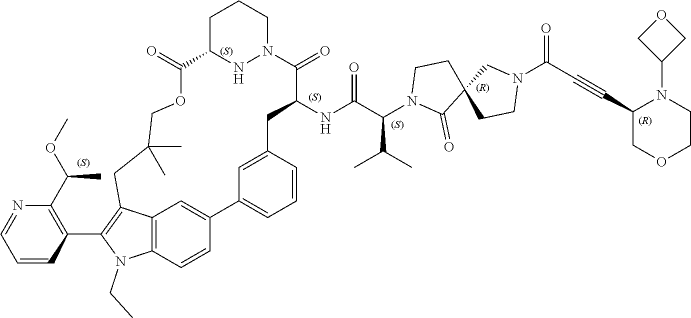 |
| A567 | 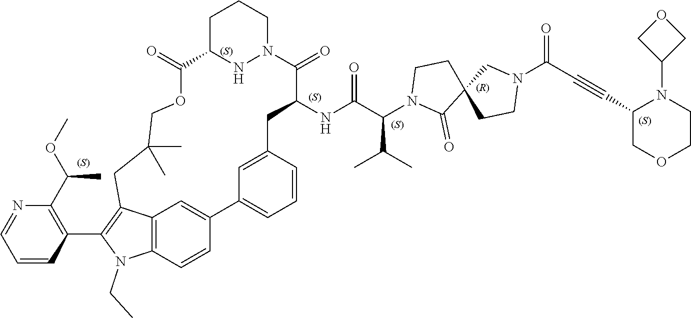 |
| A568 | 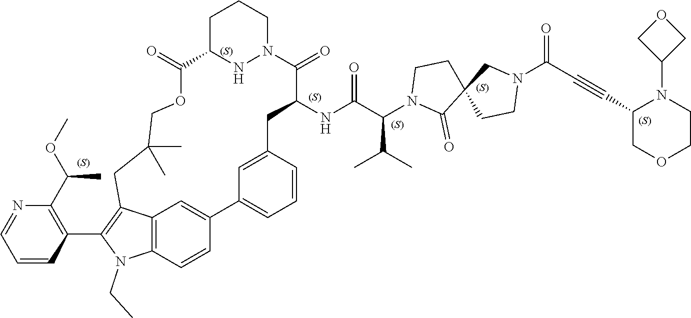 |
| A569 | 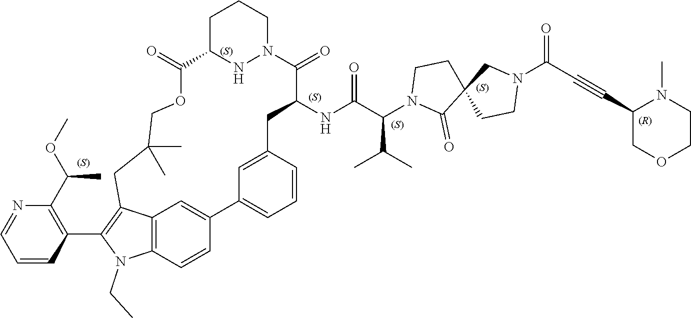 |
| A570 | 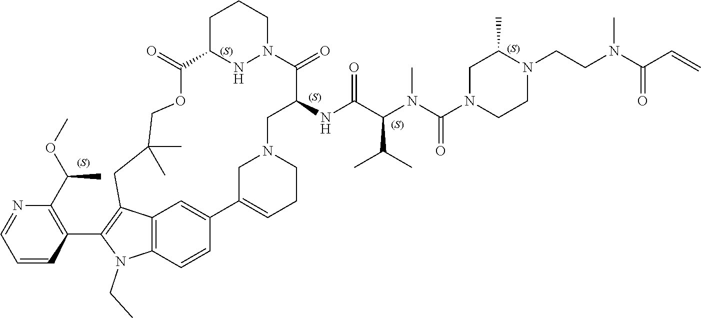 |
| A571 | 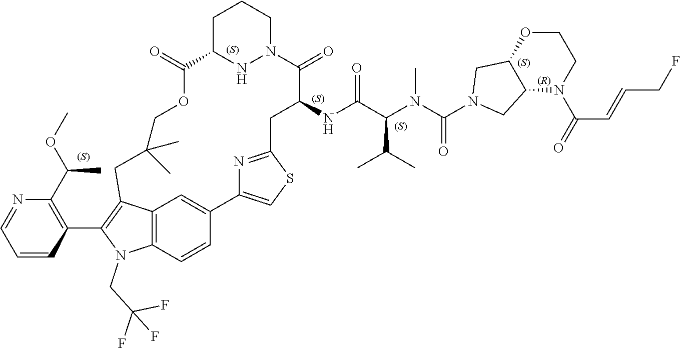 |
| A572 | 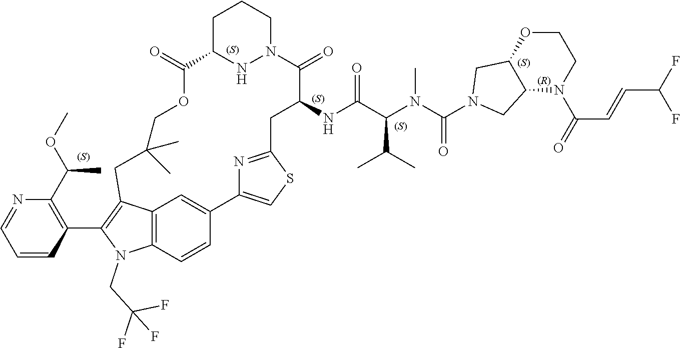 |
| A573 | 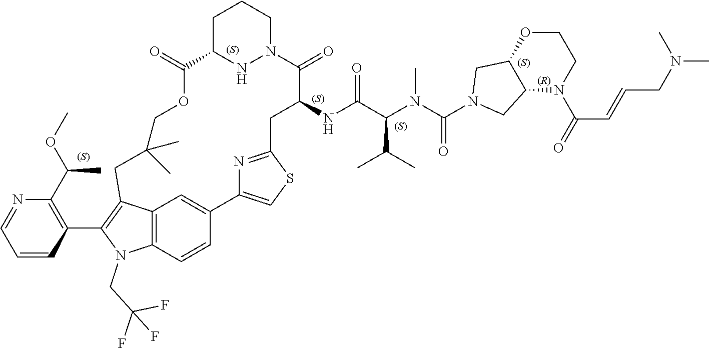 |
| A574 | 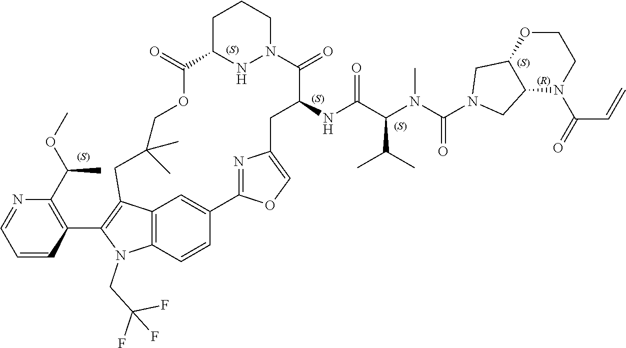 |
| A575 | 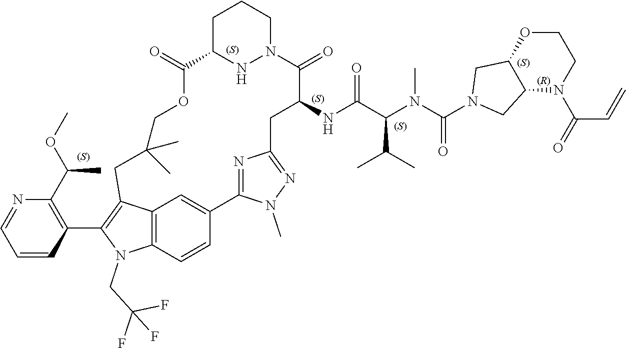 |
| A576 | 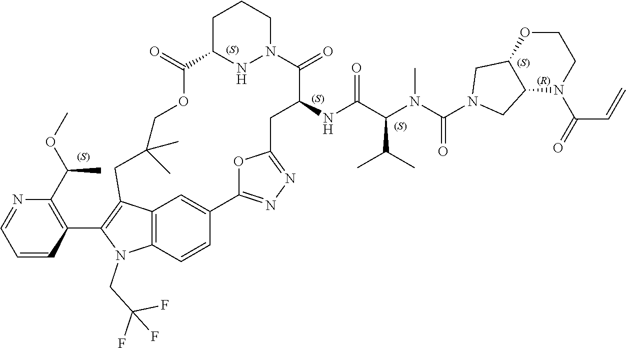 |
| A577 | 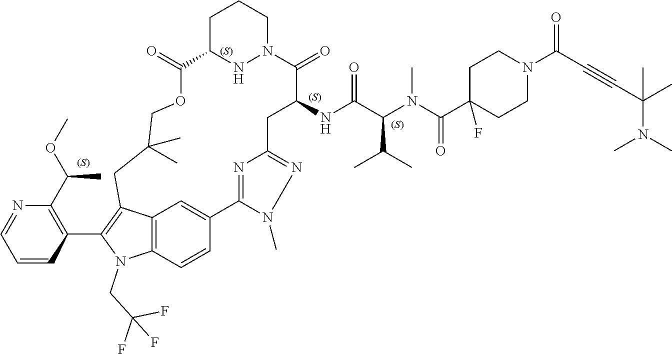 |
| A578 | 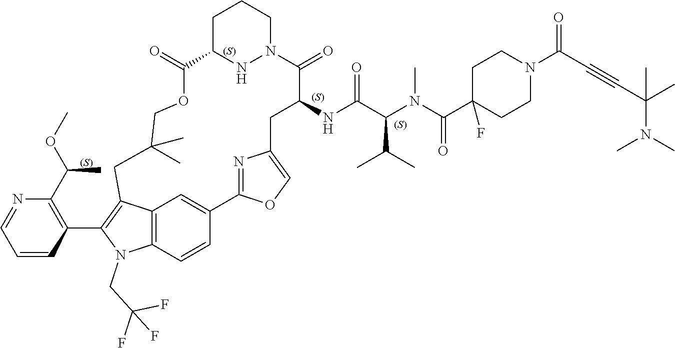 |
| A579 | 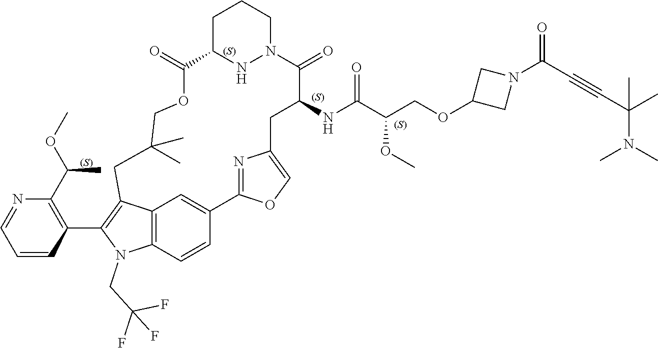 |
| A580 | 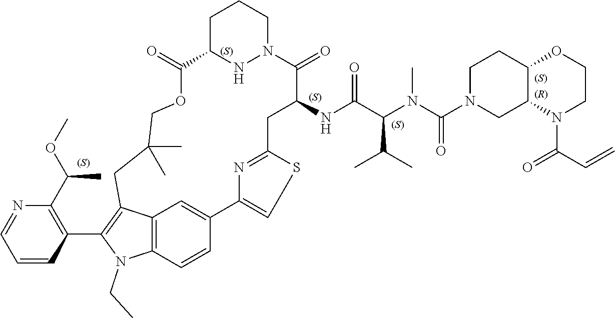 |
| A581 | 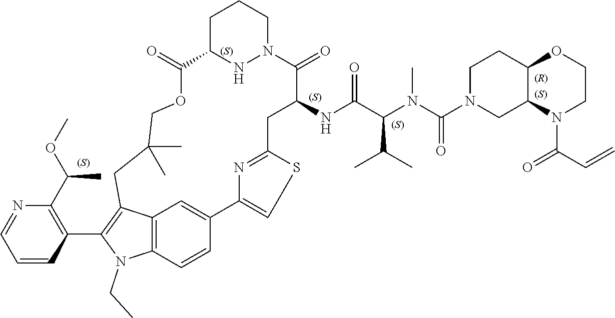 |
| A582 | 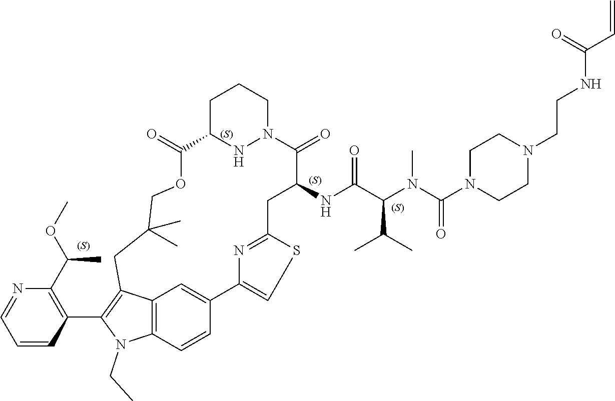 |
| A583 | 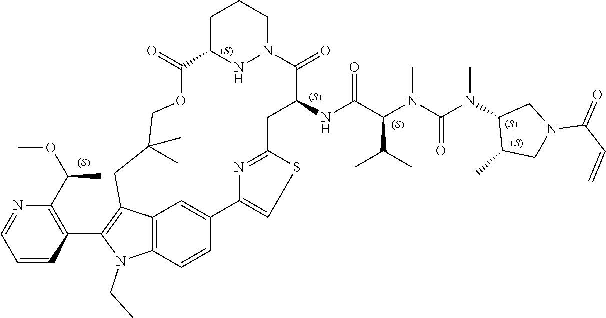 |
| A584 | 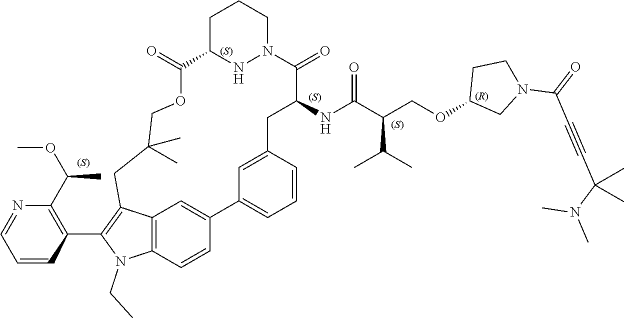 |
| A585 | 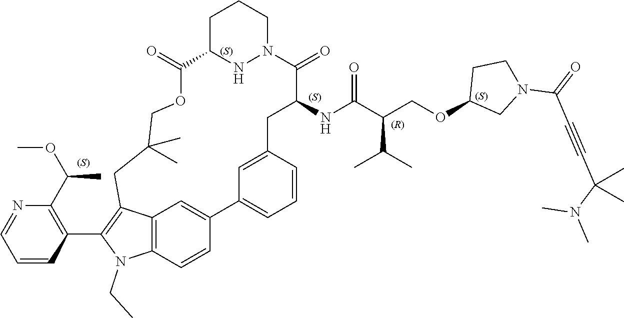 |
| A586 | 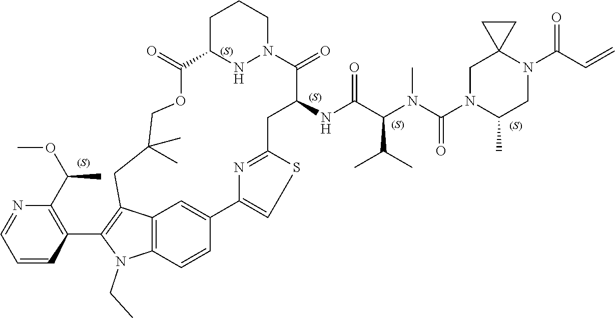 |
| A587 | 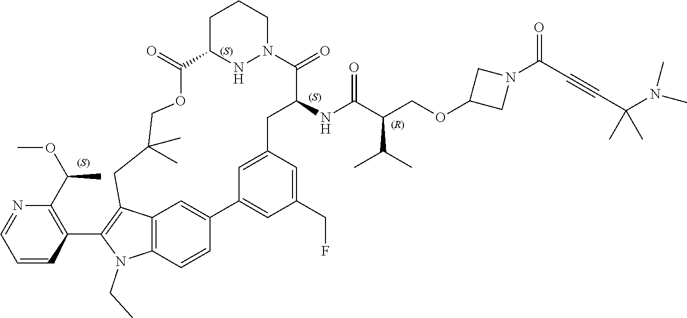 |
| A588 | 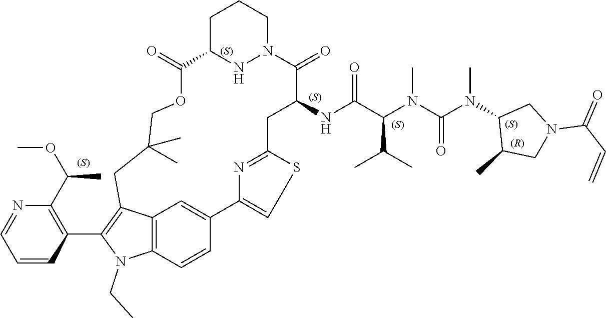 |
| A589 | 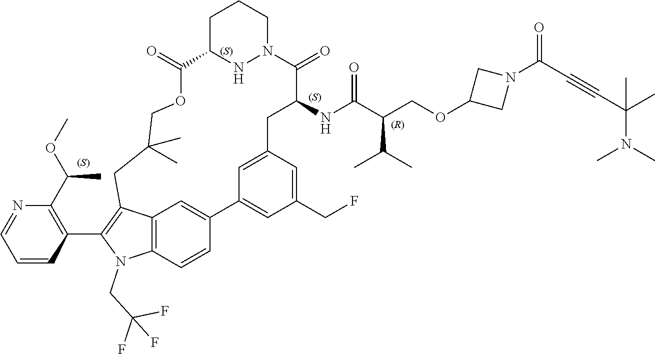 |
| A590 | 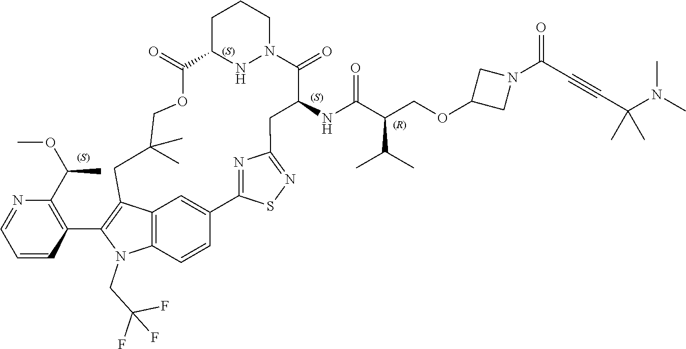 |
| A591 | 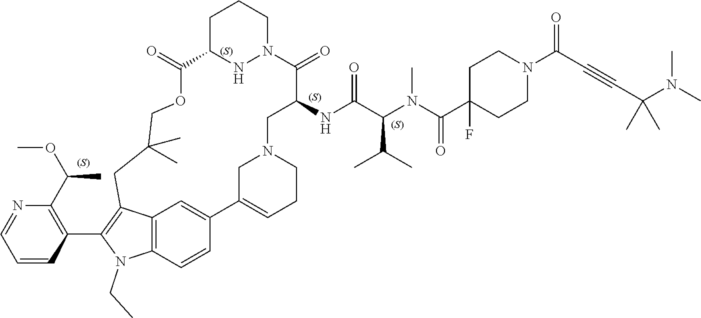 |
| A592 | 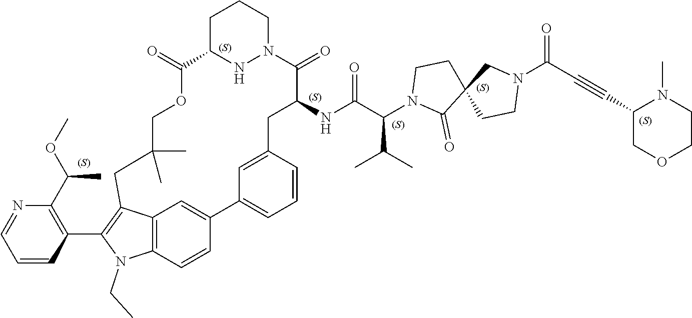 |
| A593 | 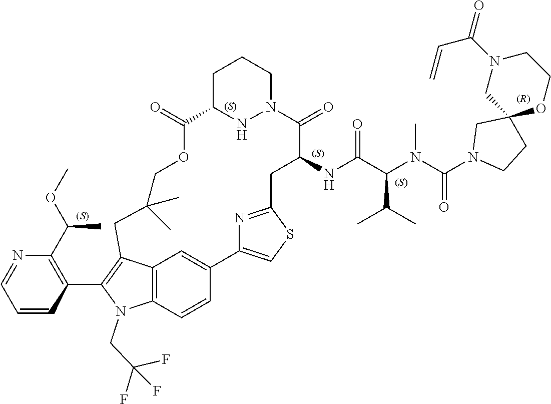 |
| A594 | 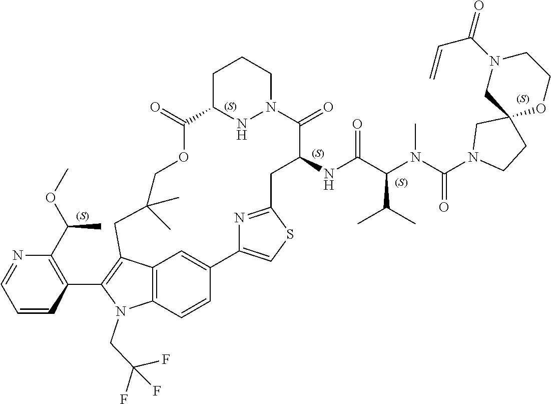 |
| A595 | 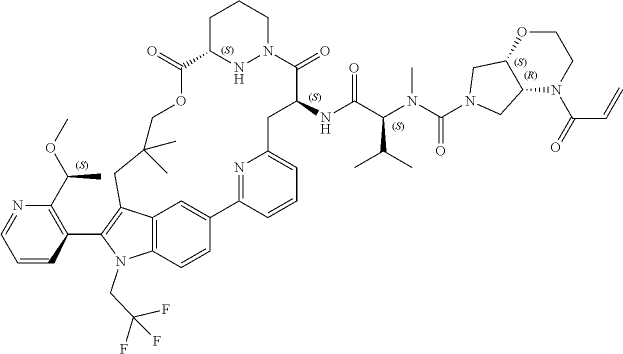 |
| A596 | 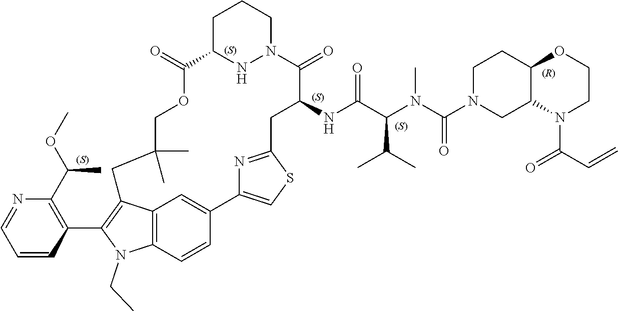 |
| A597 | 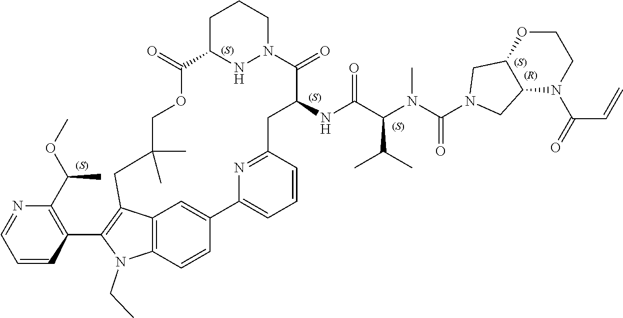 |
| A598 | 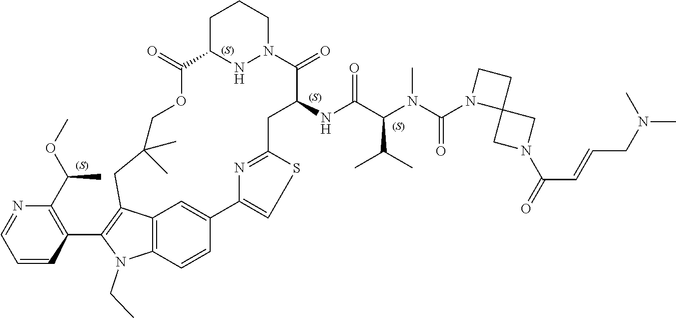 |
| A599 | 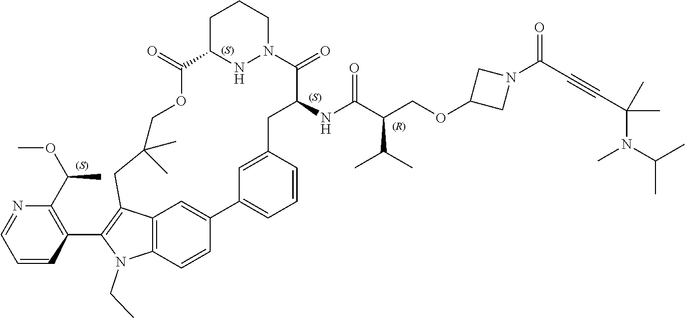 |
| A600 | 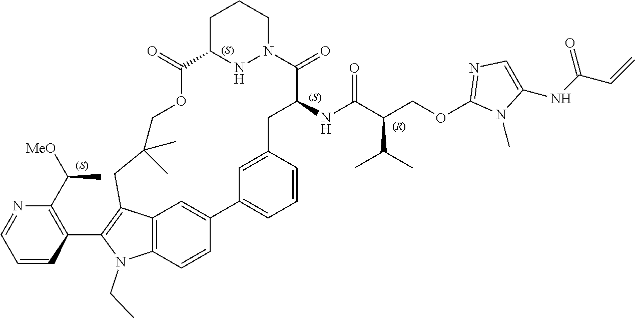 |
| A601 | 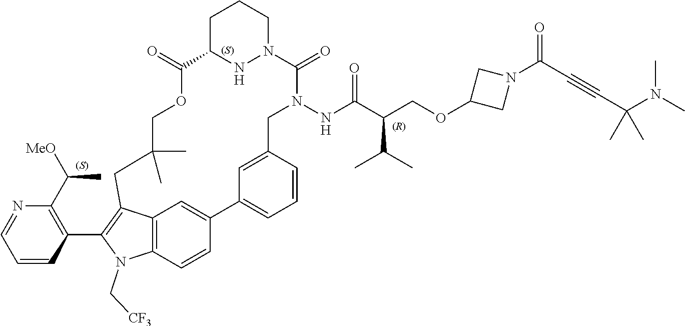 |
| A602 | 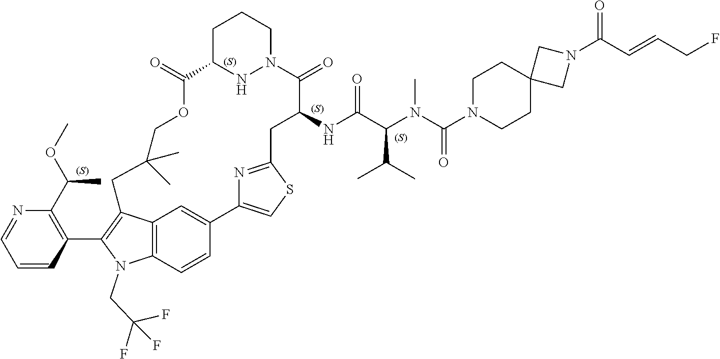 |
| A603 | 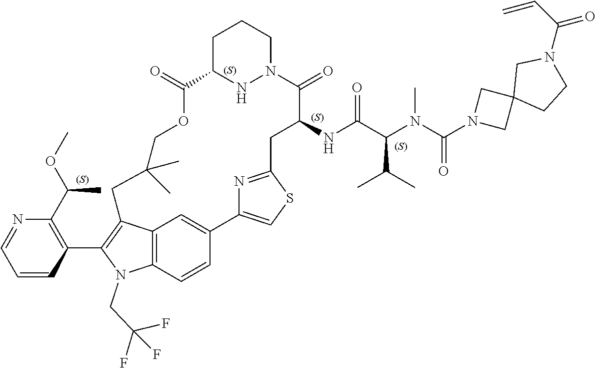 |
| A604 | 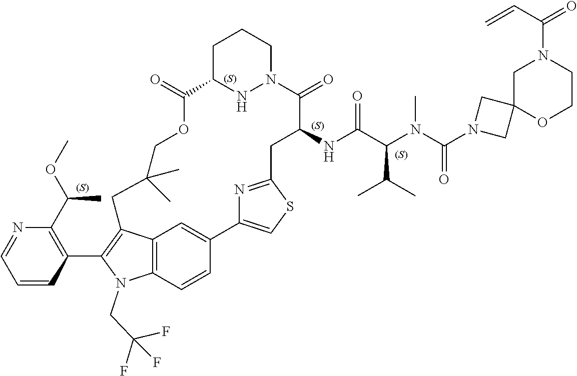 |
| A605 | 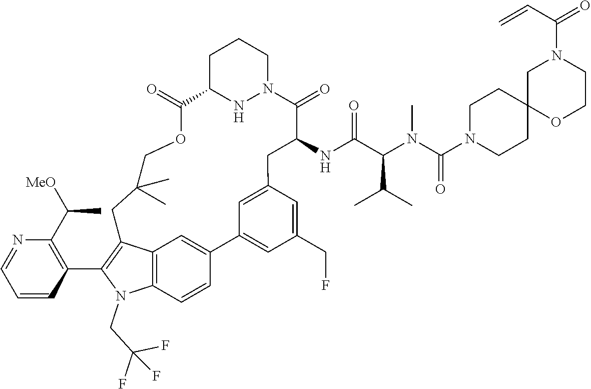 |
| A606 | 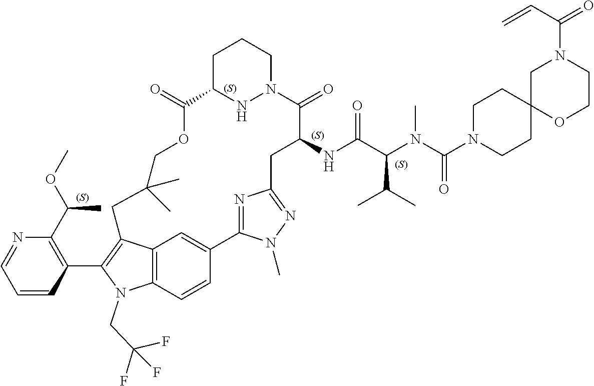 |
| A607 | 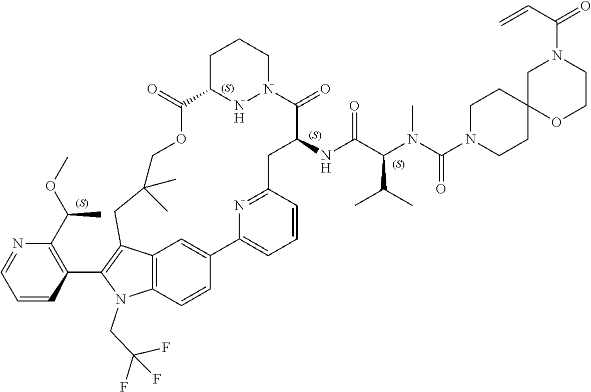 |
| A608 | 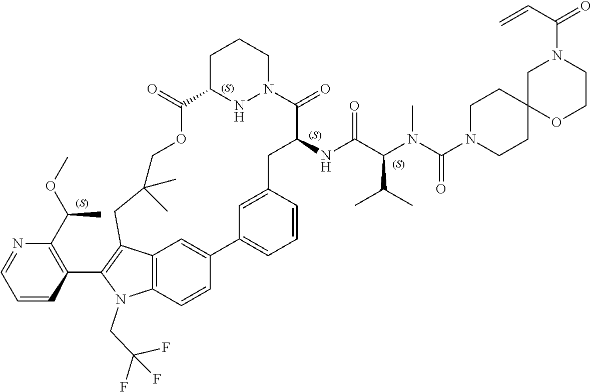 |
| A609 | 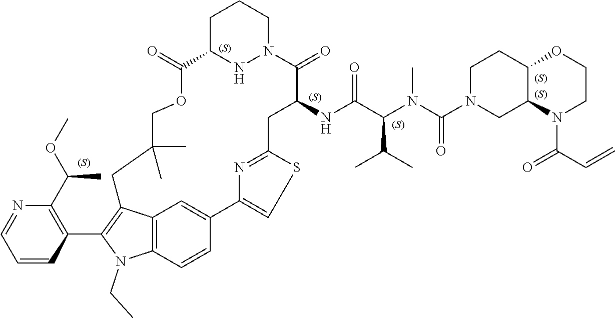 |
| A610 | 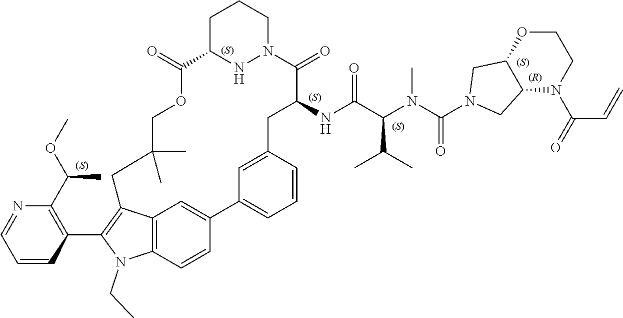 |
| A611 | 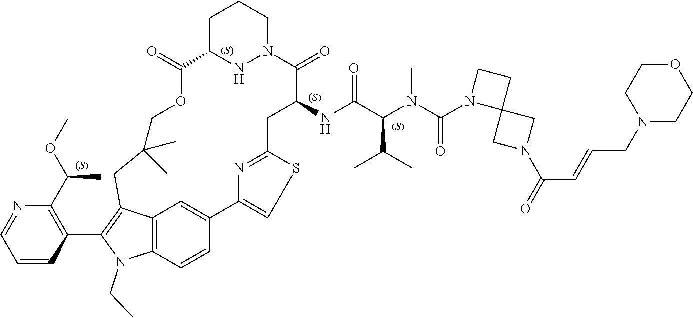 |
| A612 | 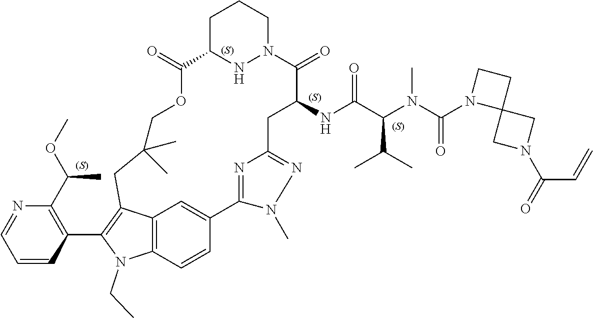 |
| A613 | 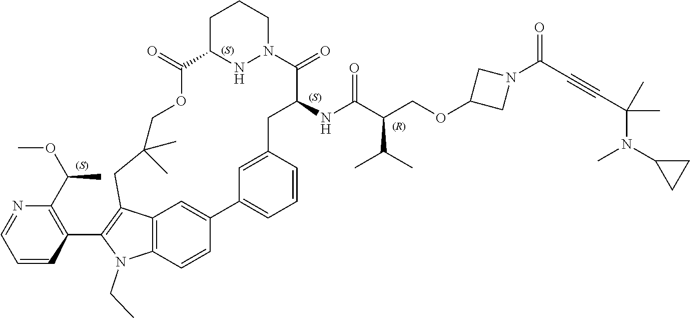 |
| A614 | 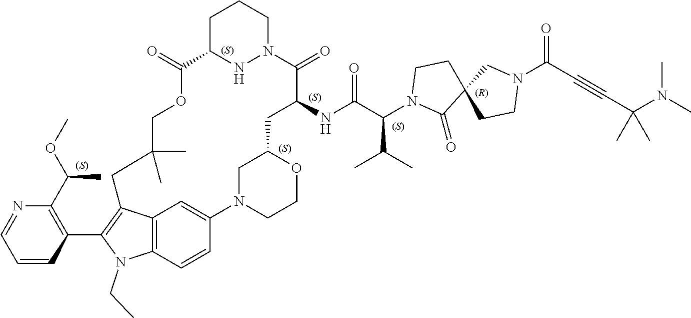 |
| A615 | 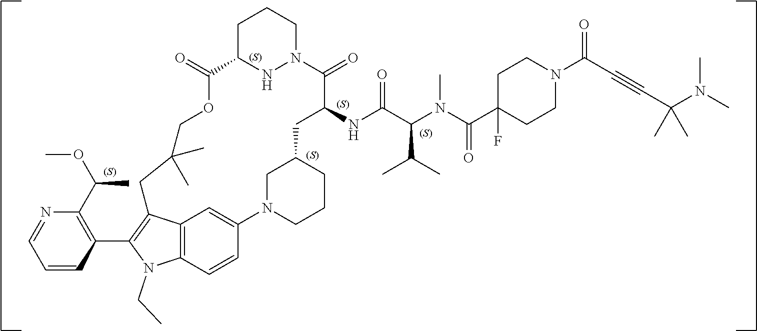 |
| A616 | 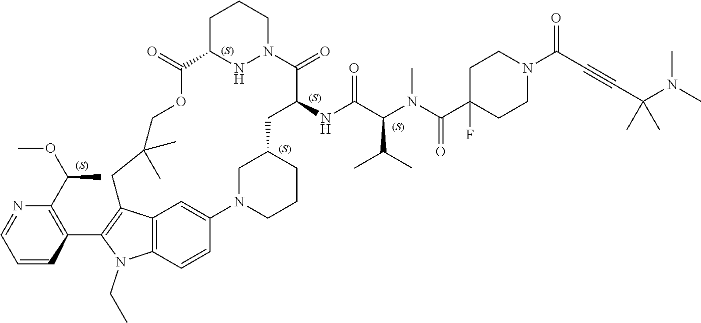 |
| A617 | 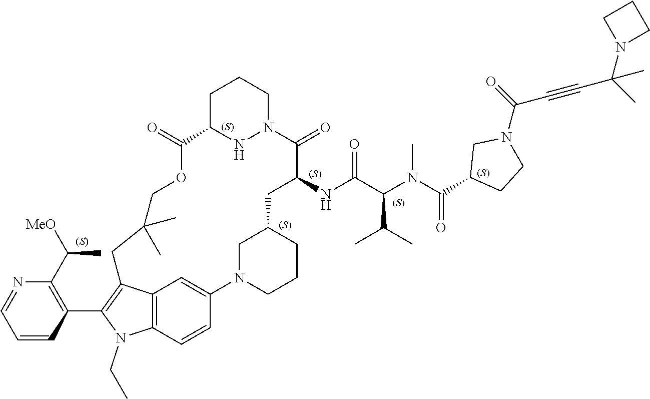 |
| A618 | 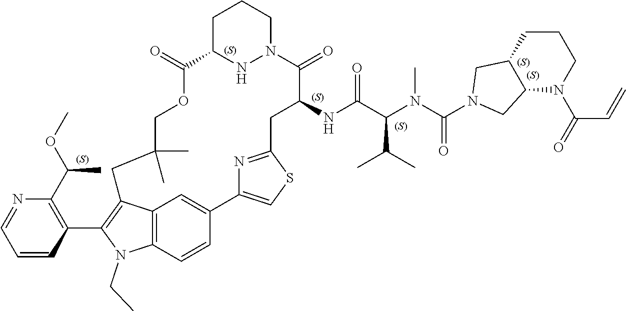 |
| A619 | 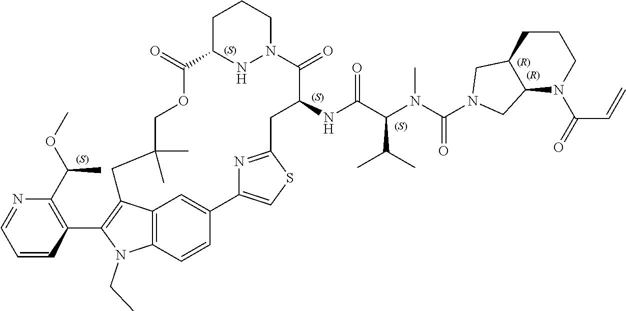 |
| A620 | 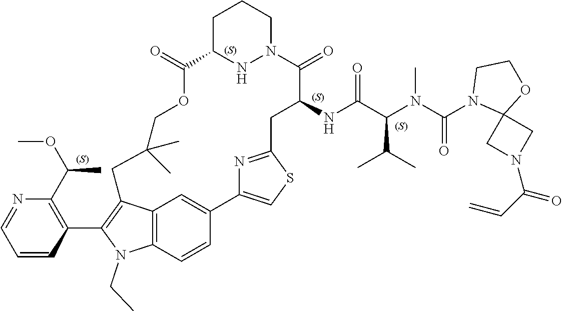 |
| A621 | 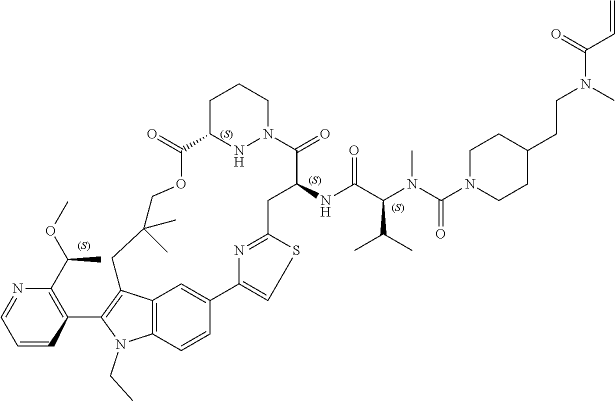 |
| A622 | 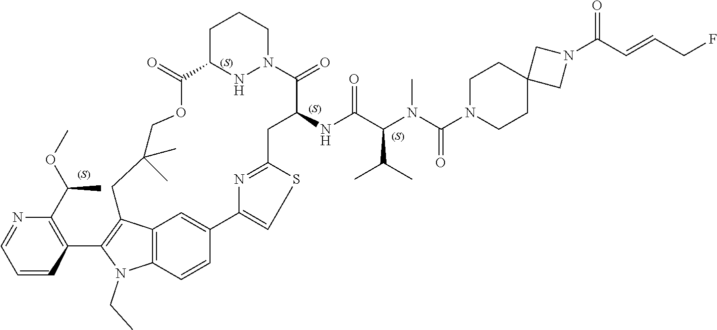 |
| A623 | 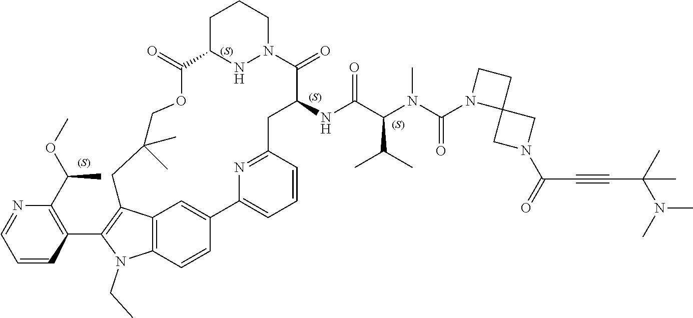 |
| A624 | 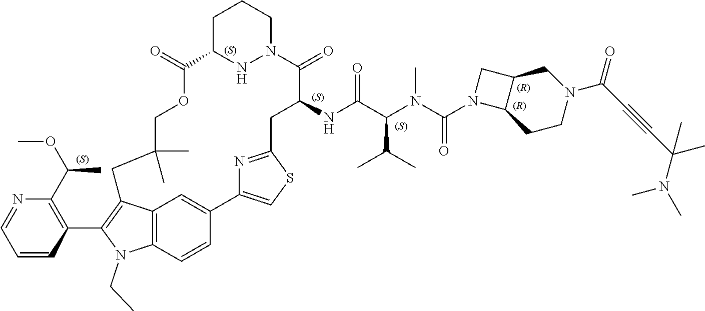 |
| A625 | 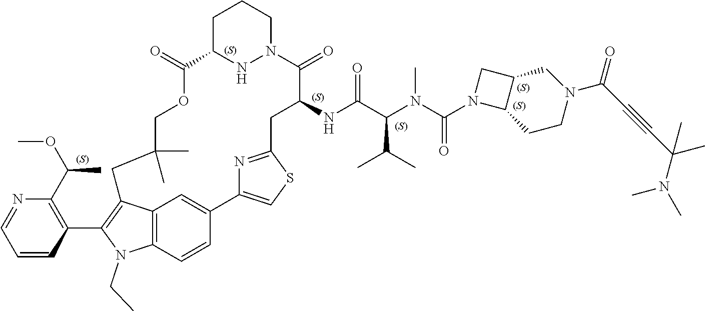 |
| A626 | 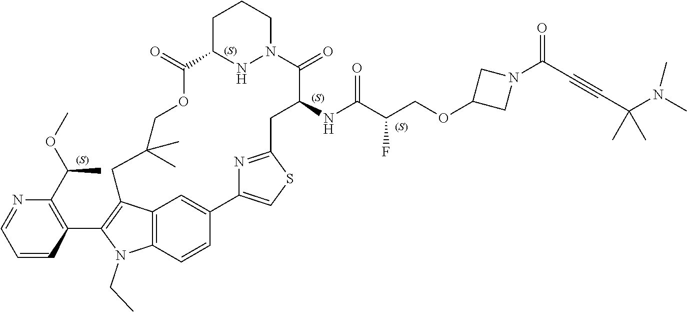 |
| A627 | 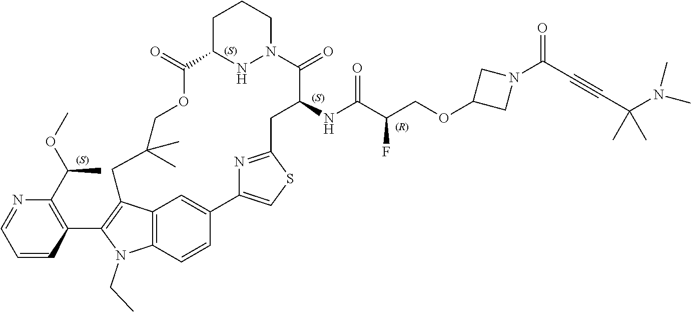 |
| A628 | 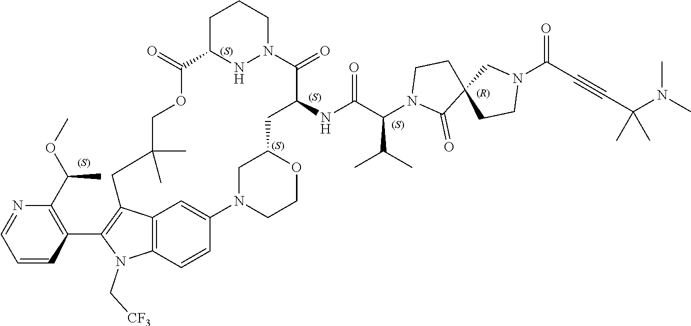 |
| A629 | 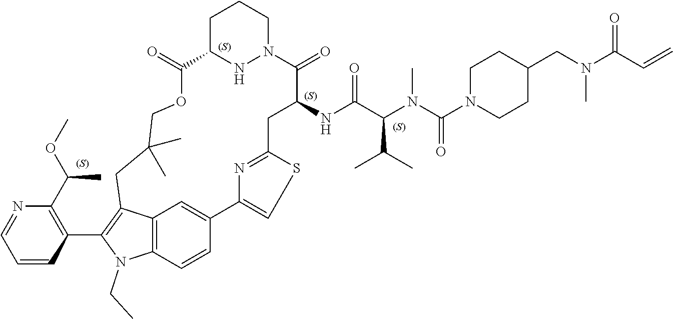 |
| A630 | 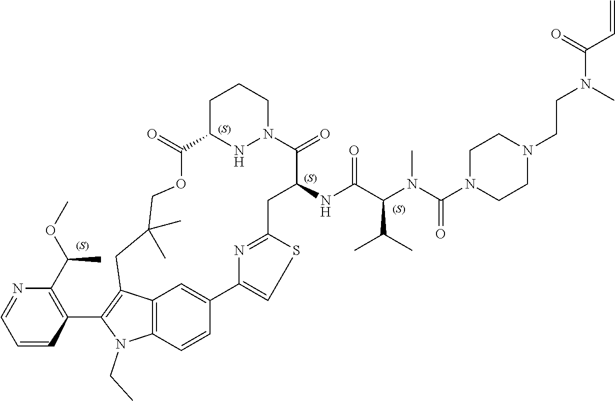 |
| A631 | 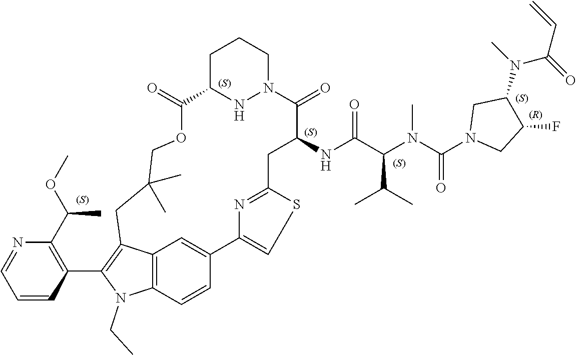 |
| A632 | 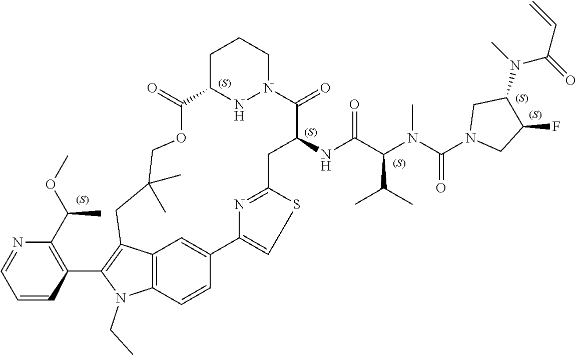 |
| A633 | 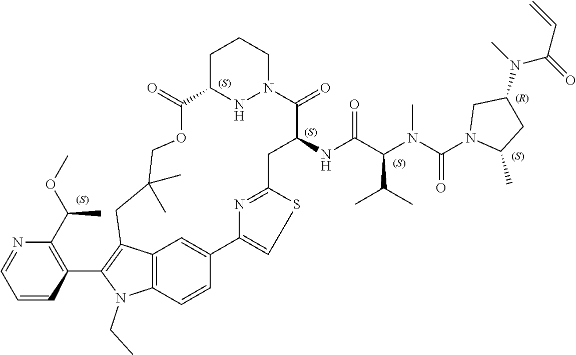 |
| A634 | 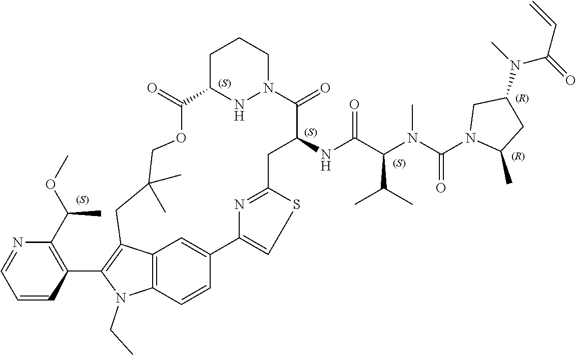 |
| A635 | 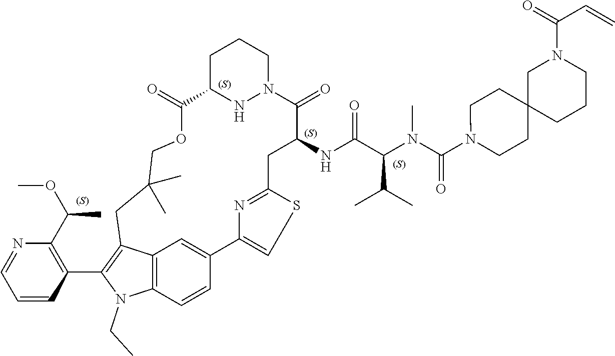 |
| A636 | 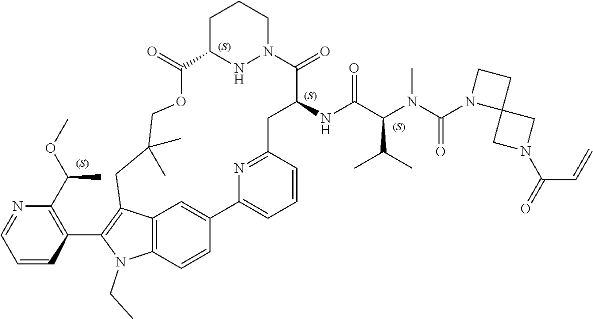 |
| A637 | 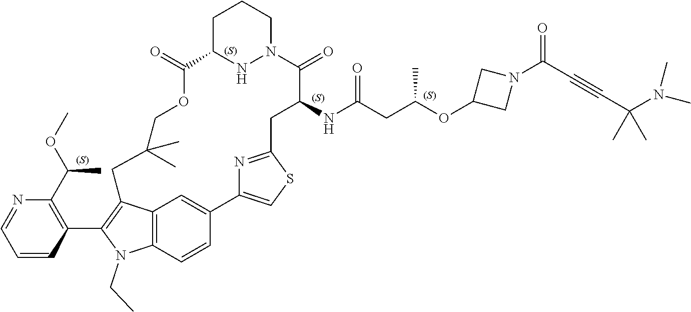 |
| A638 | 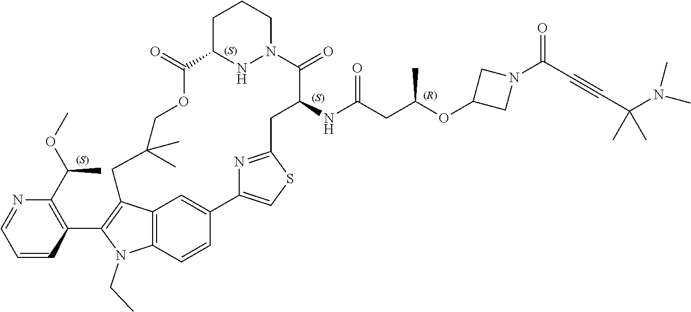 |
| A639 | 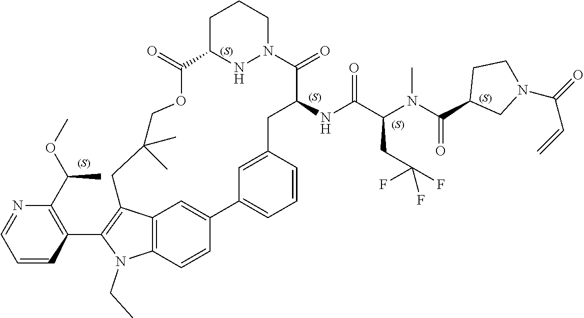 |
| A640 | 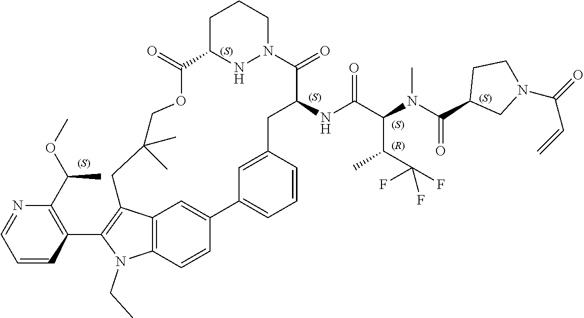 |
| A641 | 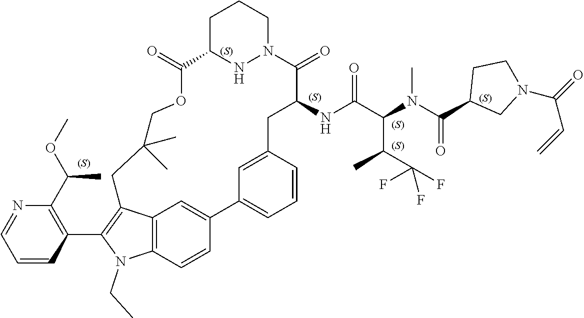 |
| A642 | 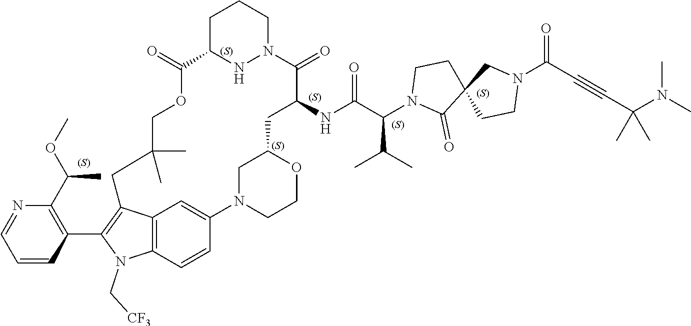 |
| A643 | 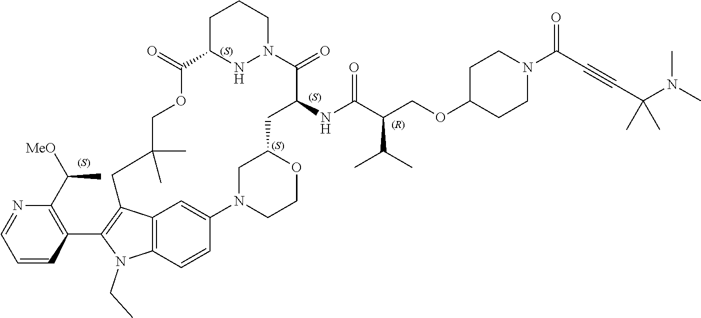 |
| A644 | 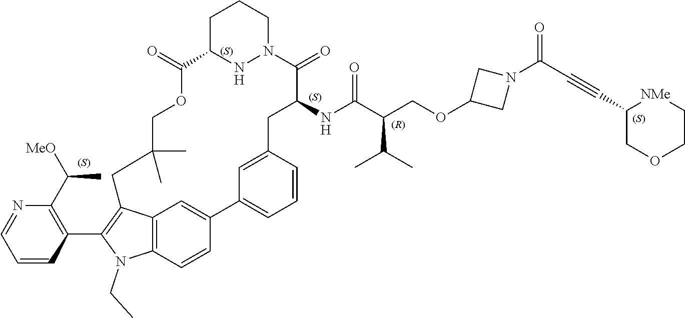 |
| A645 | 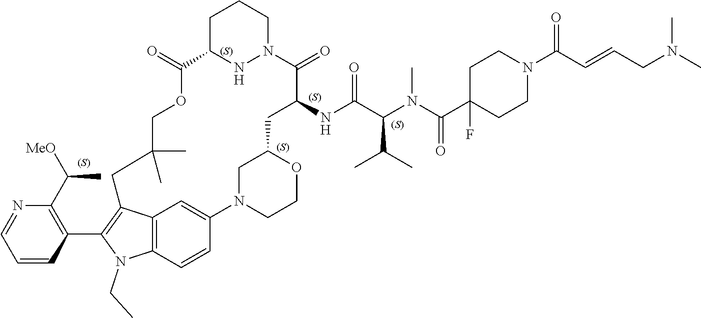 |
| A646 | 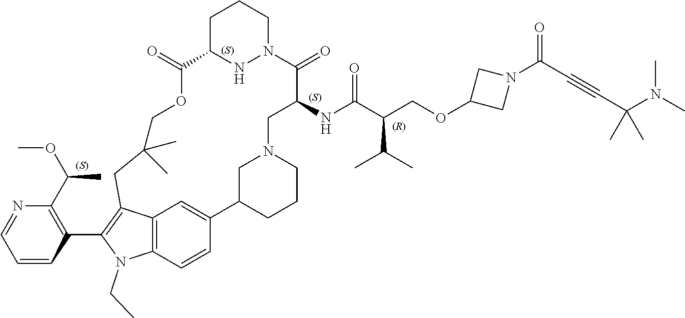 |
| A647 | 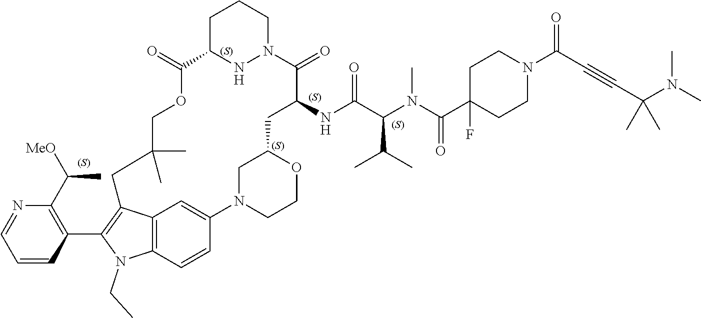 |
| A648 | 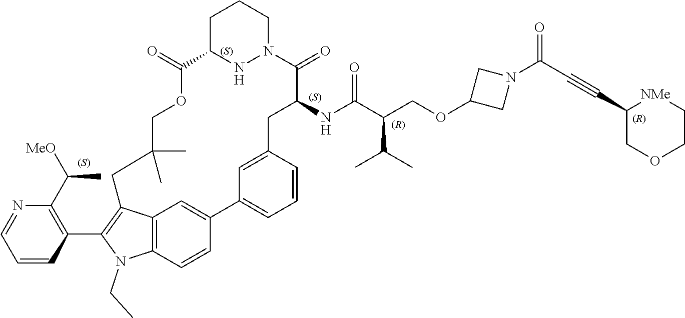 |
| A649 | 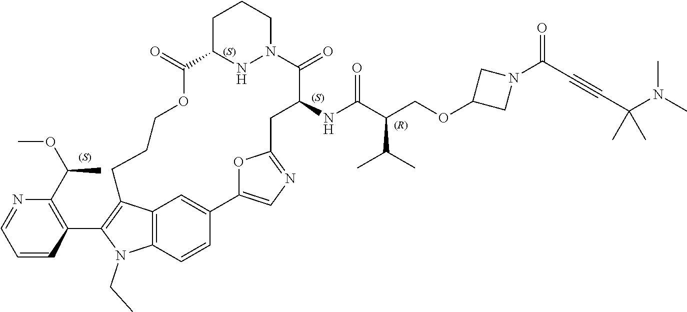 |
| A650 | 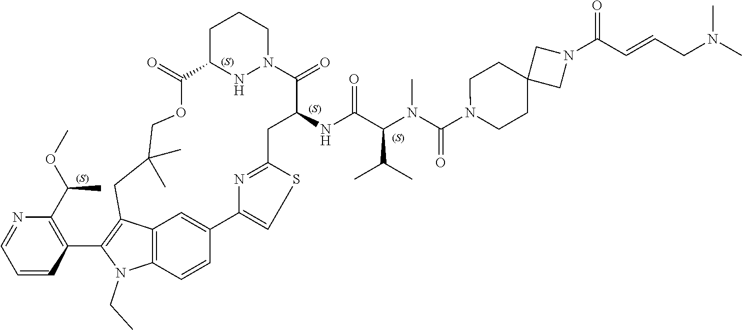 |
| A651 | 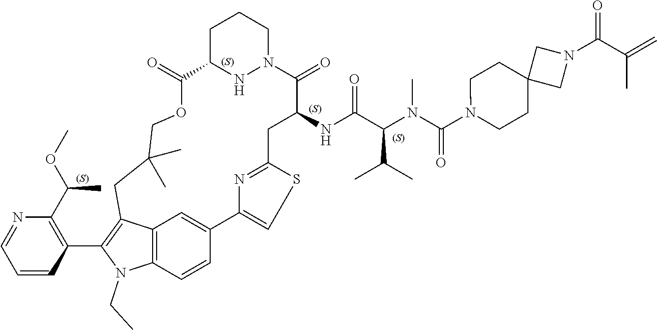 |
| A652 | 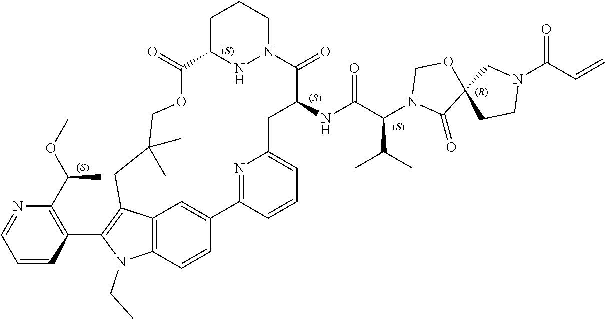 |
| A653 | 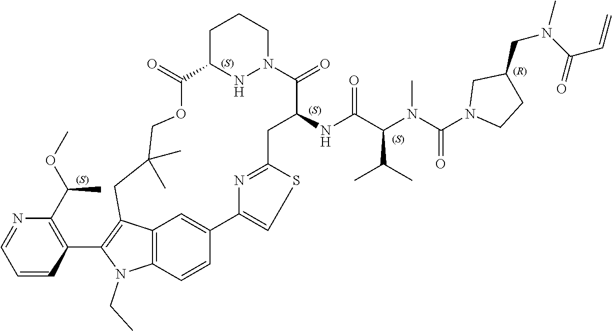 |
| A654 | 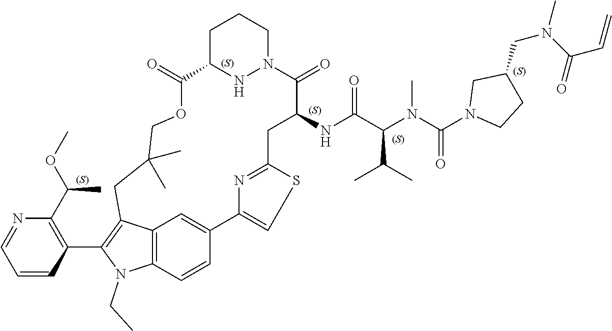 |
| A655 | 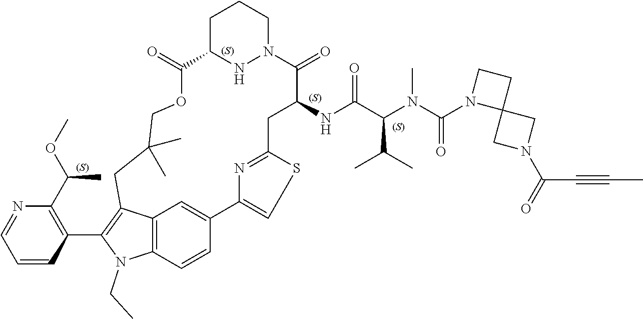 |
| A656 | 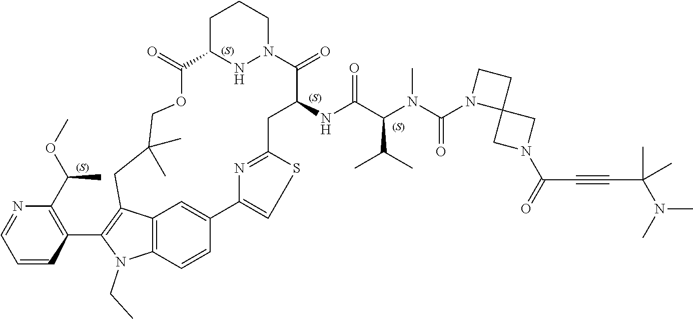 |
| A657 | 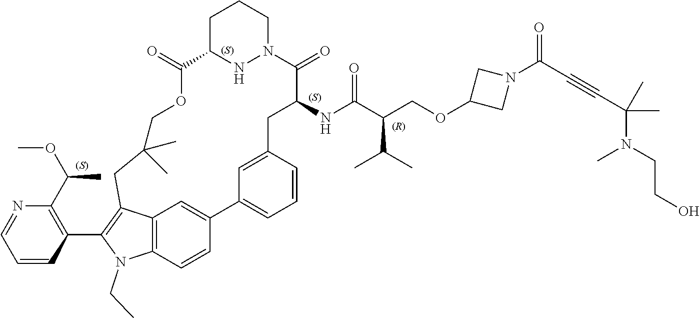 |
| A658 | 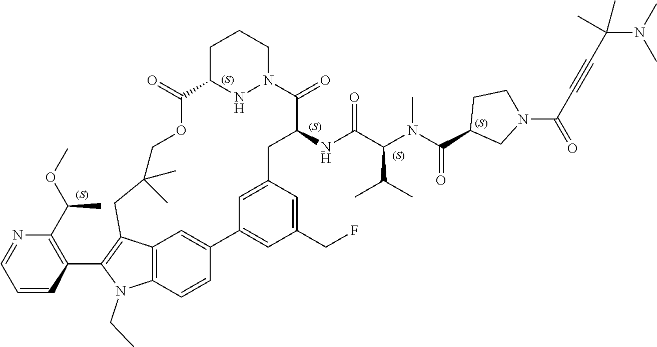 |
| A659 | 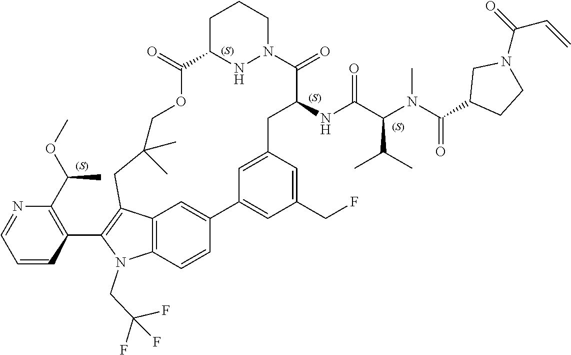 |
| A660 | 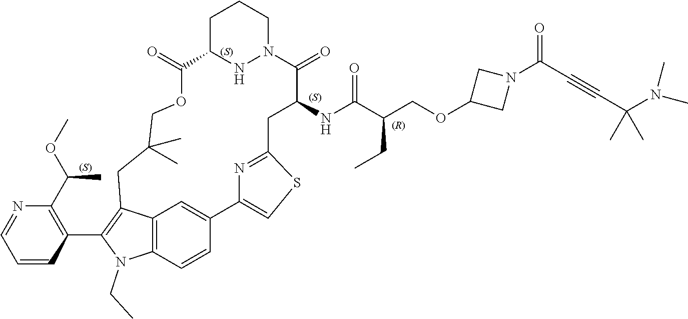 |
| A661 | 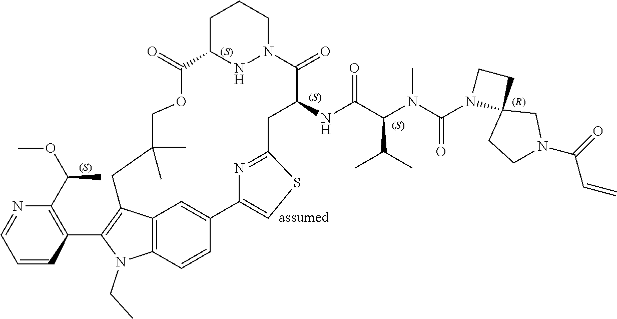 |
| A662 | 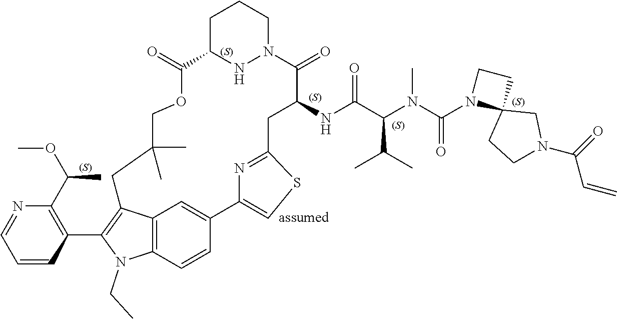 |
| A663 | 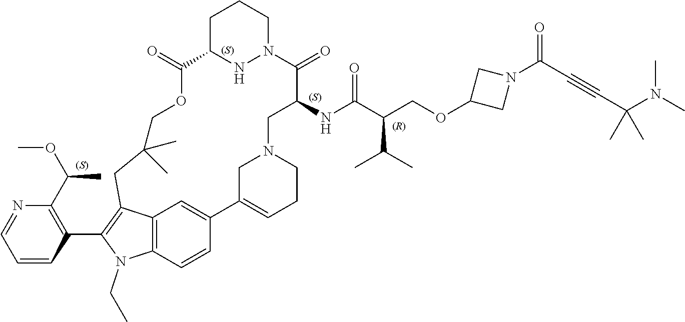 |
| A664 | 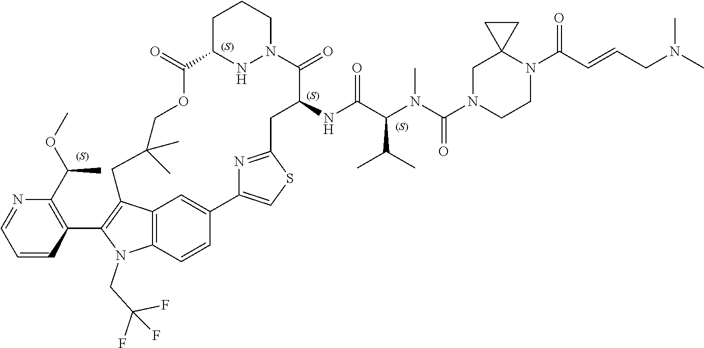 |
| A665 | 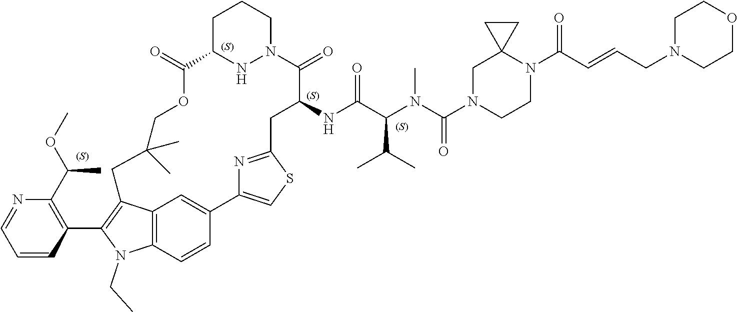 |
| A666 | 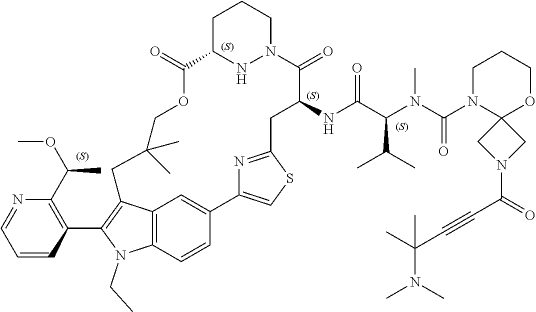 |
| A667 | 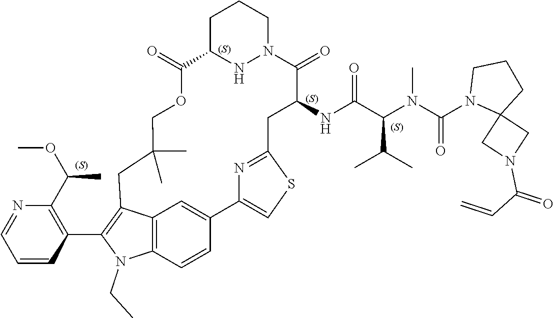 |
| A668 | 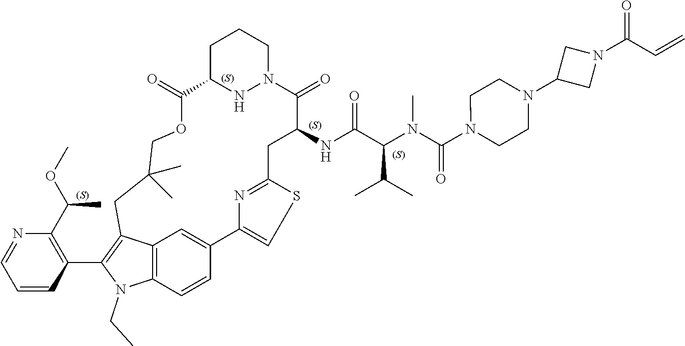 |
| A669 | 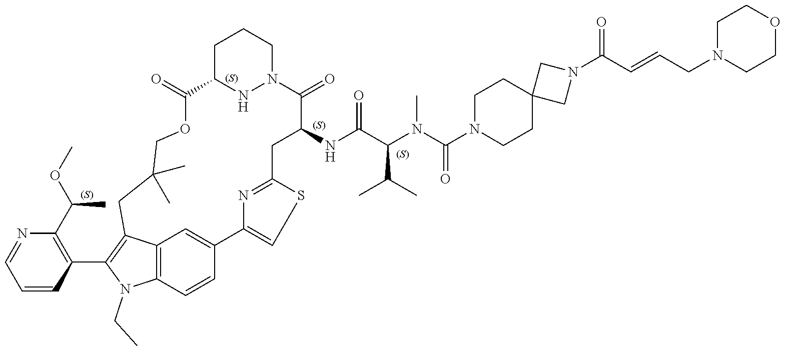 |
| A670 | 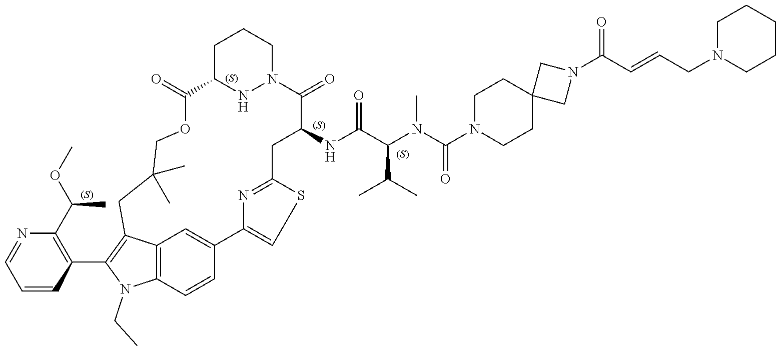 |
| A671 | 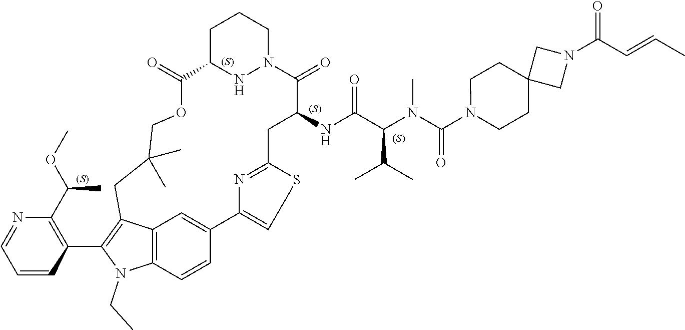 |
| A672 | 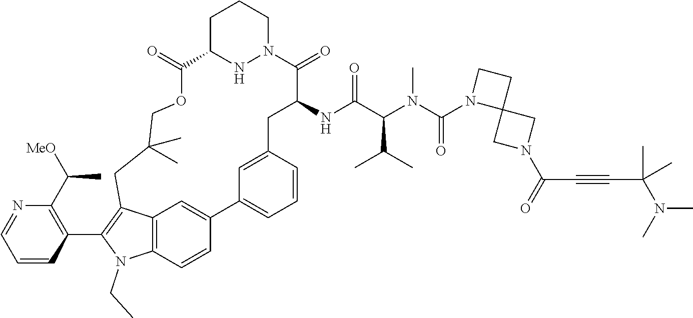 |
| A673 | 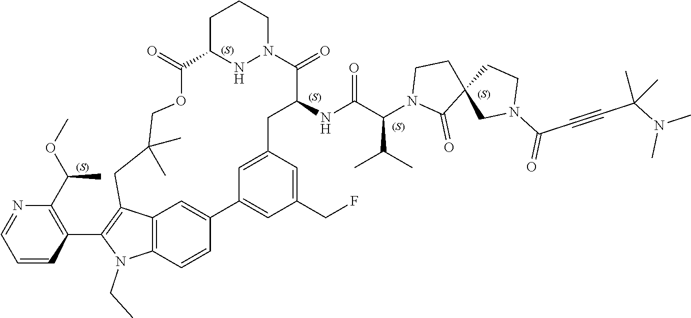 |
| A674 | 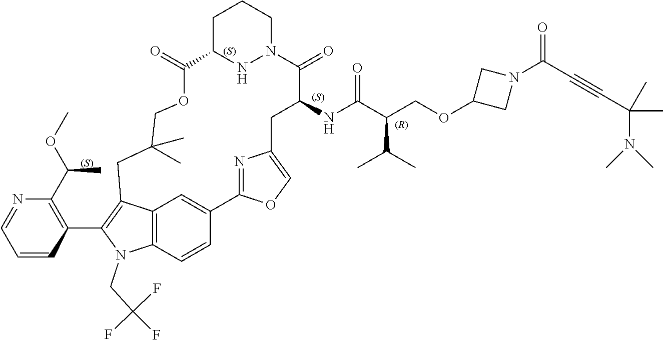 |
| A675 | 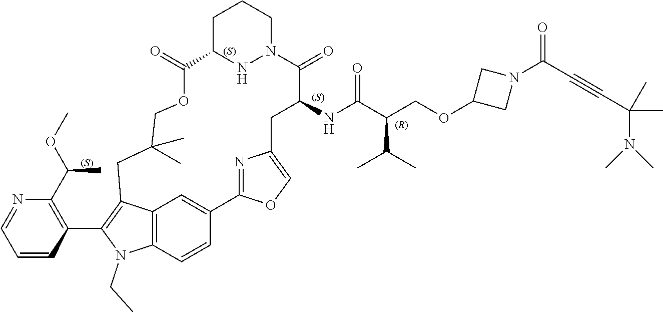 |
| A676 | 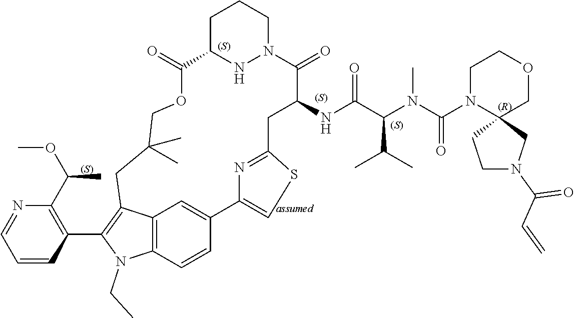 |
| A677 | 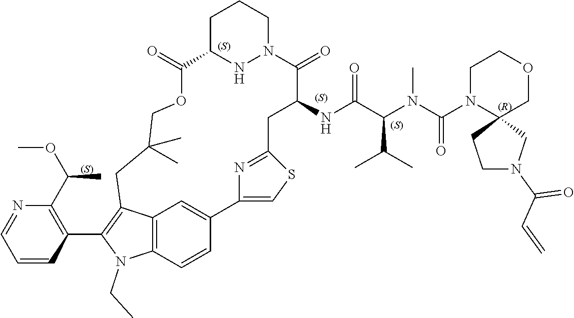 |
| A678 | 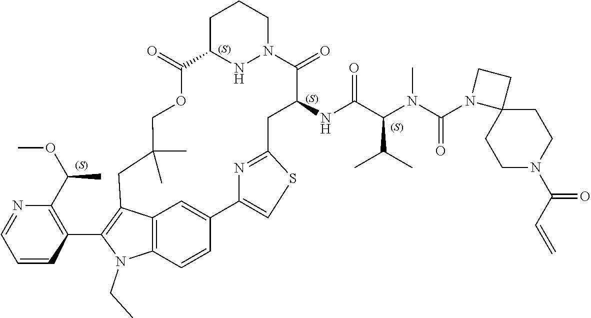 |
| A679 | 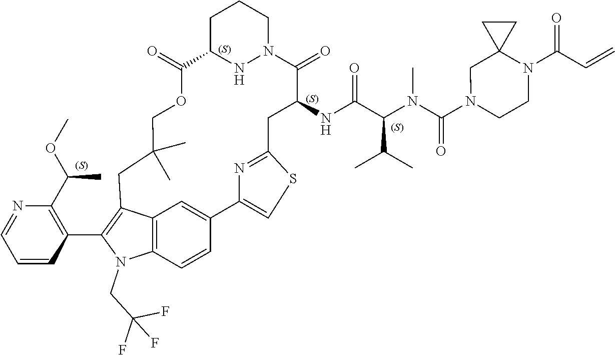 |
| A680 | 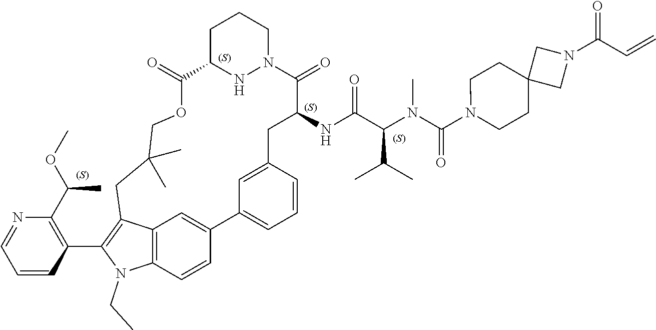 |
| A681 | 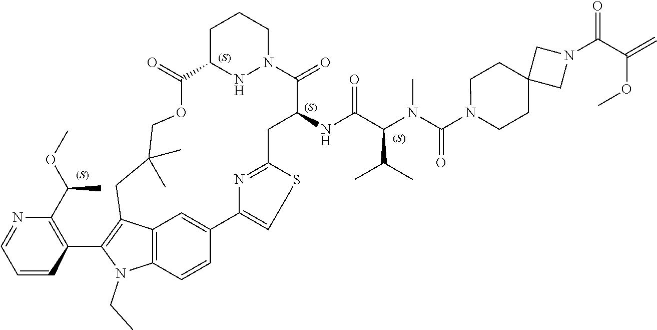 |
| A682 | 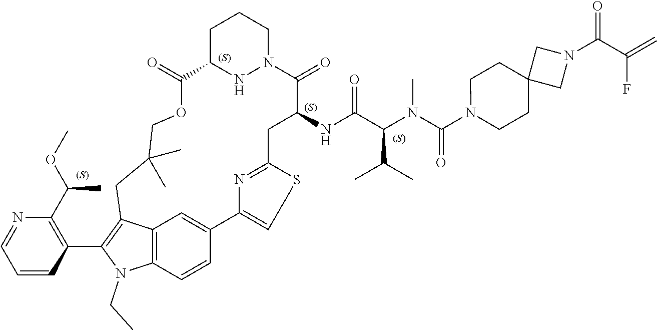 |
| A683 | 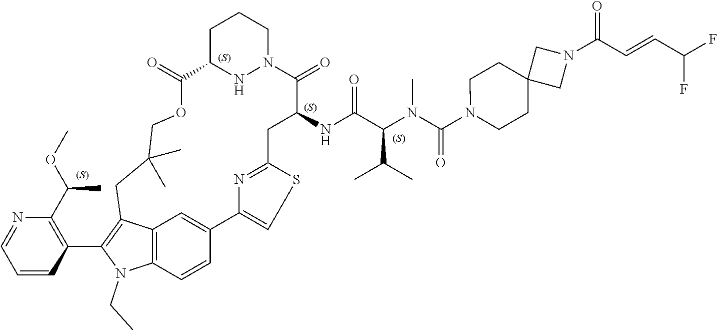 |
| A684 | 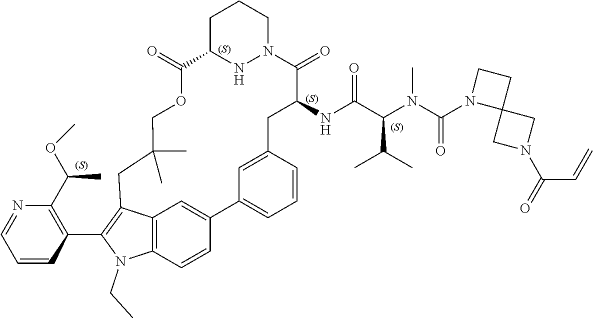 |
| A685 | 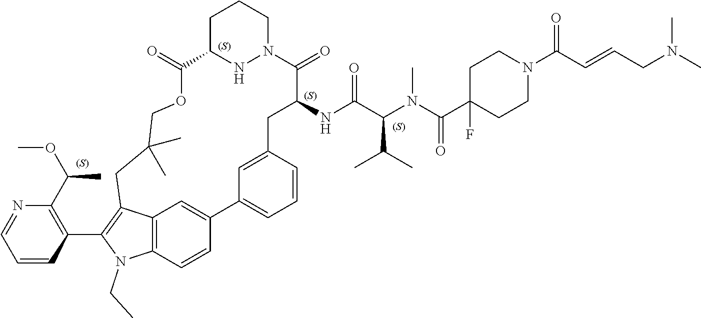 |
| A686 | 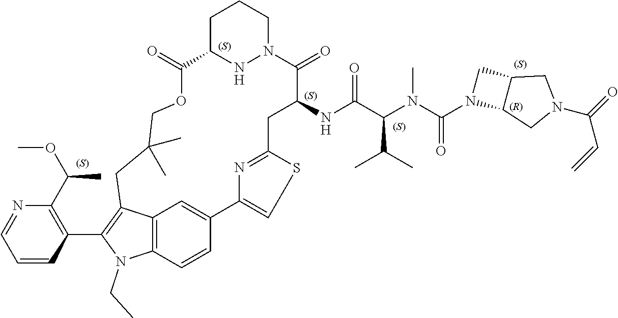 |
| A687 | 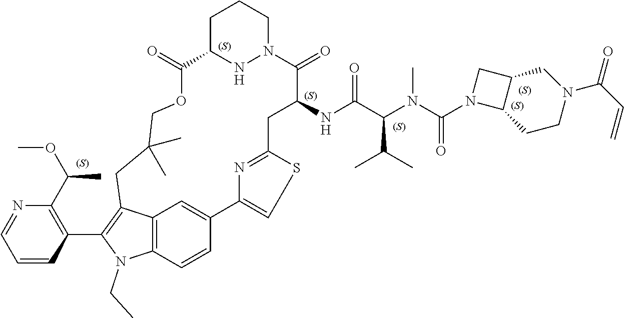 |
| A688 | 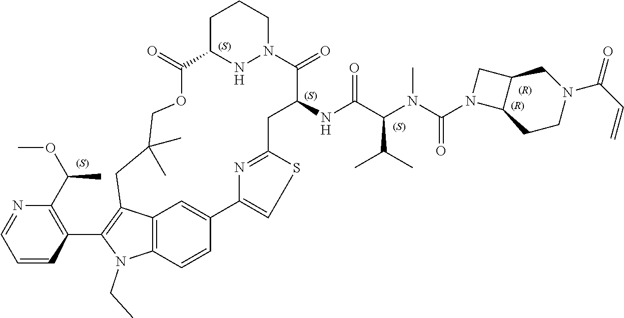 |
| A689 | 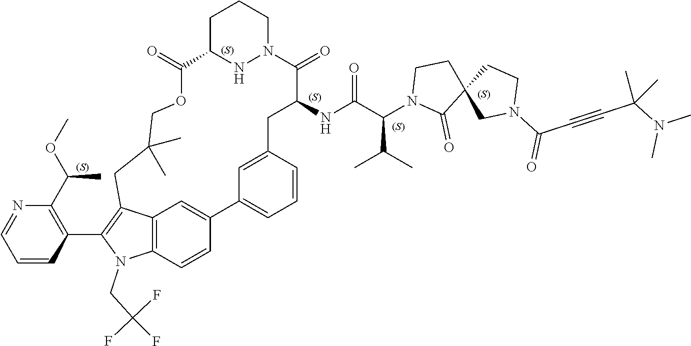 |
| A690 | 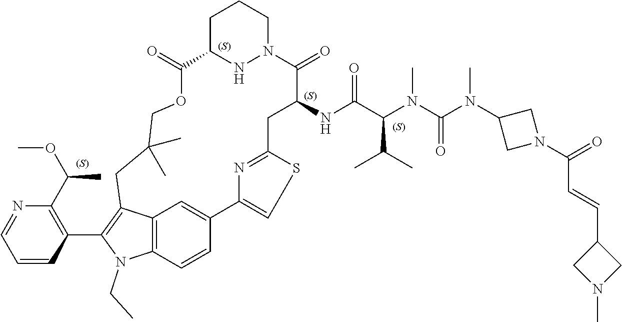 |
| A691 | 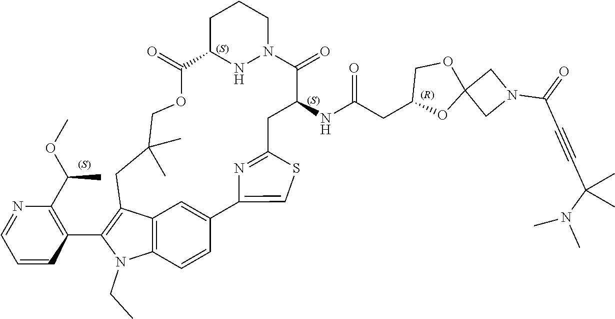 |
| A692 | 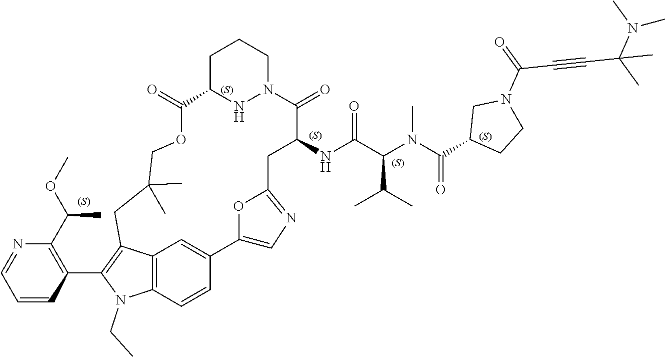 |
| A693 | 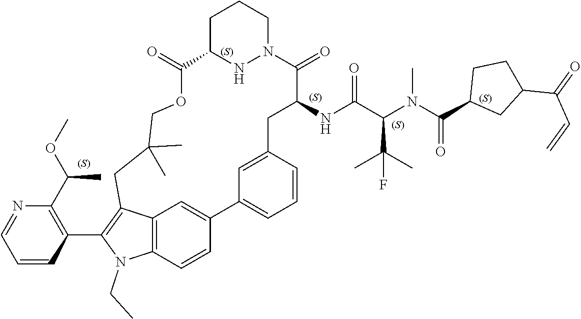 |
| A694 | 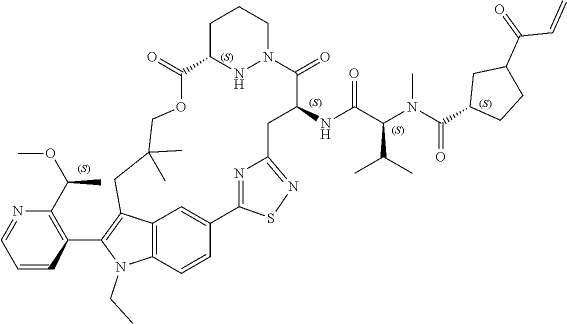 |
| A695 | 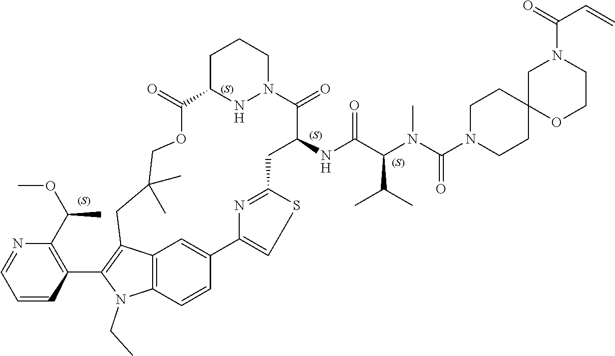 |
| A696 | 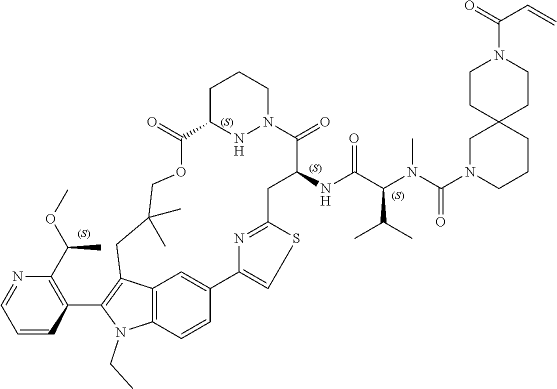 |
| A697 | 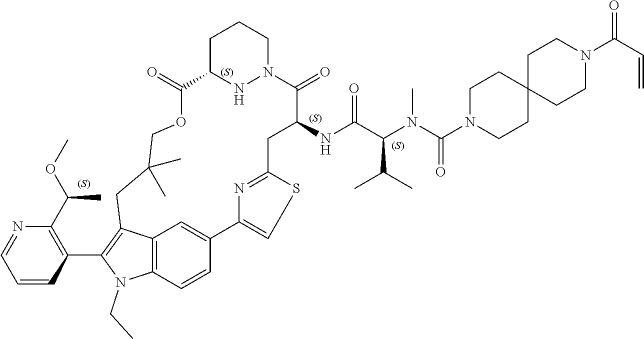 |
| A698 | 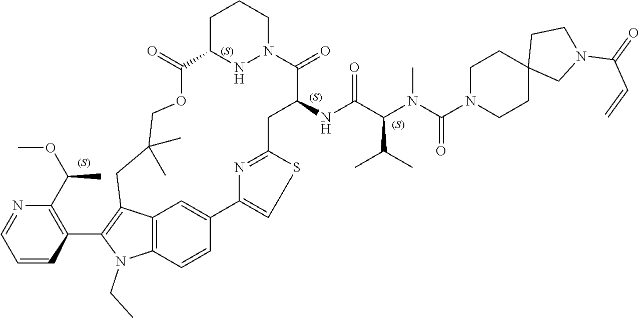 |
| A699 | 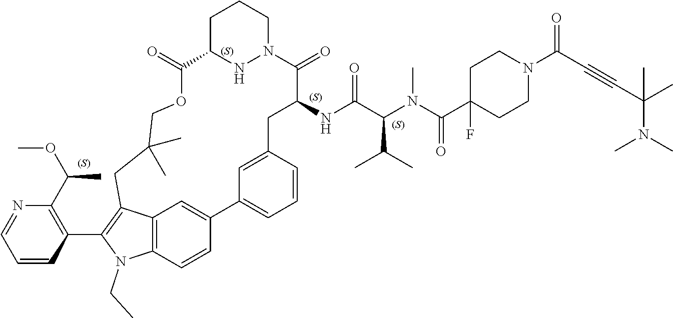 |
| A700 | 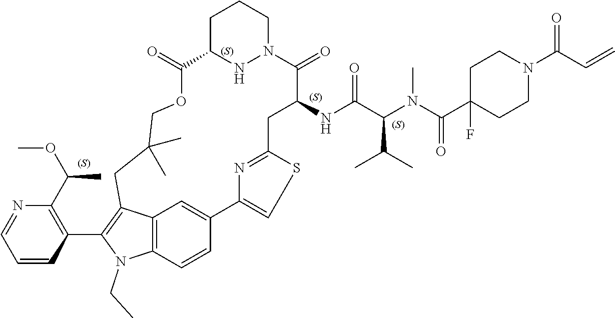 |
| A701 | 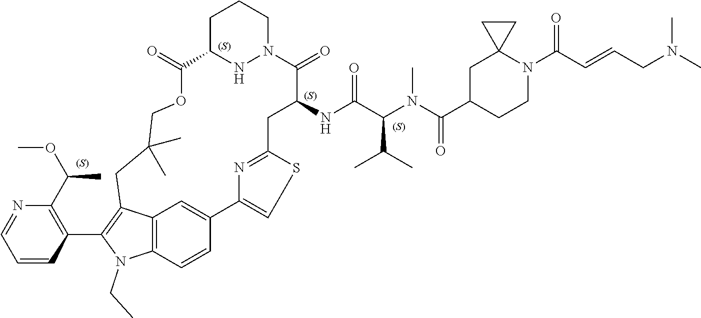 |
| A702 | 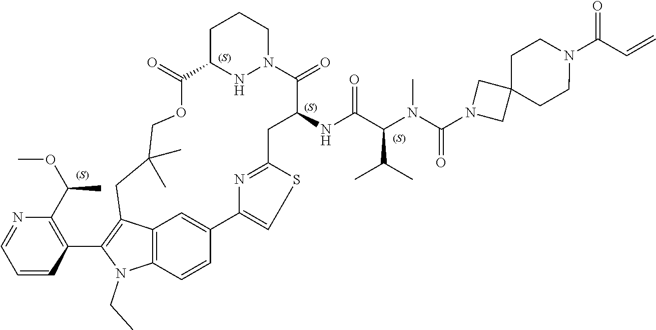 |
| A703 | 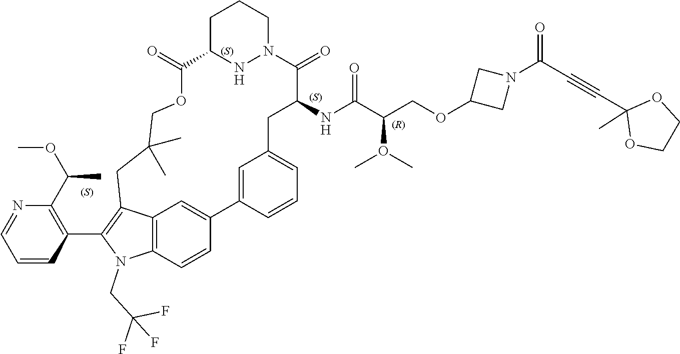 |
| A704 | 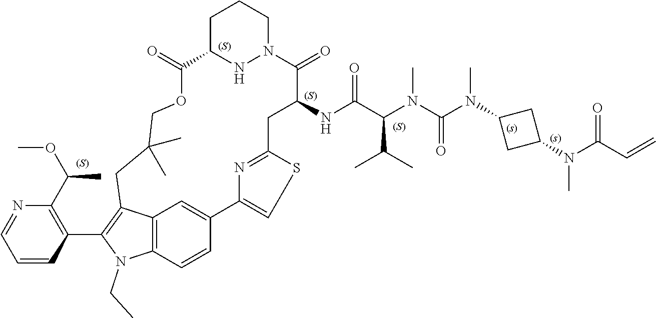 |
| A705 | 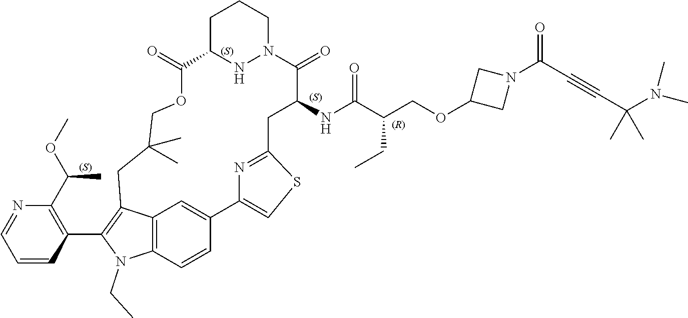 |
| A706 | 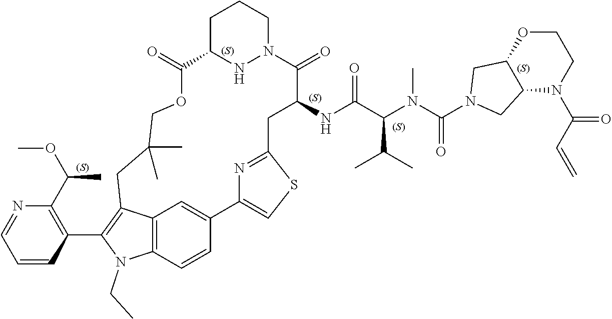 |
| A707 | 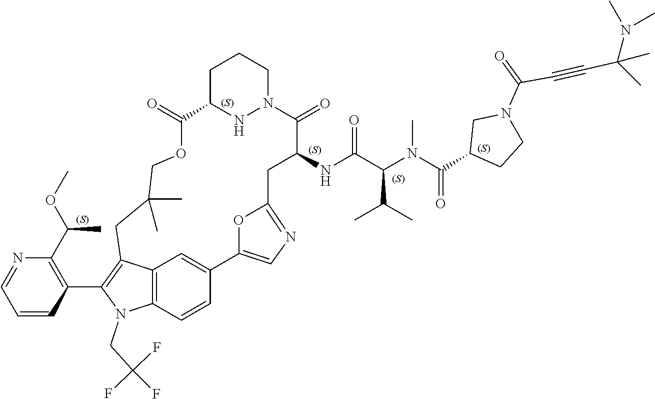 |
| A708 | 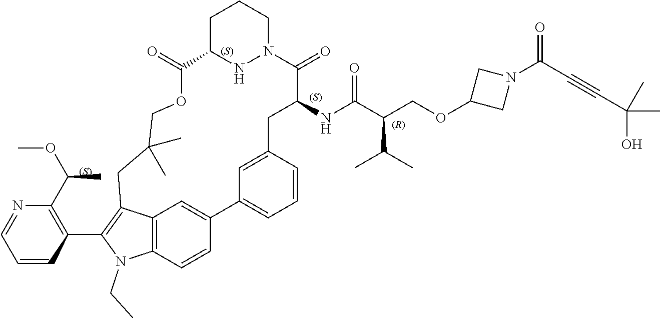 |
| A709 | 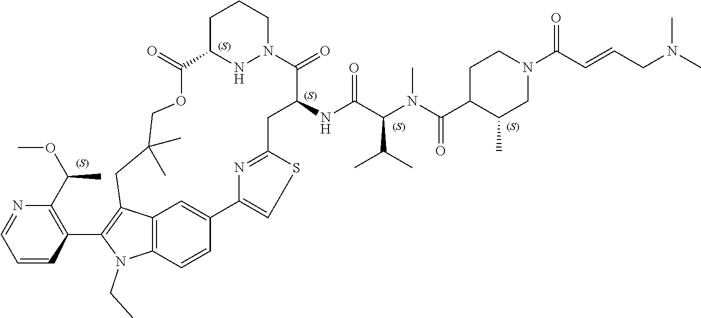 |
| A710 | 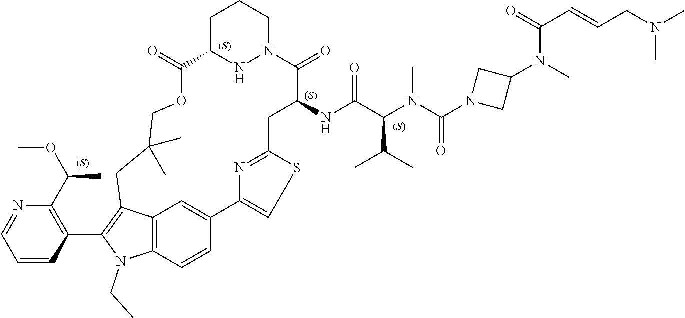 |
| A711 | 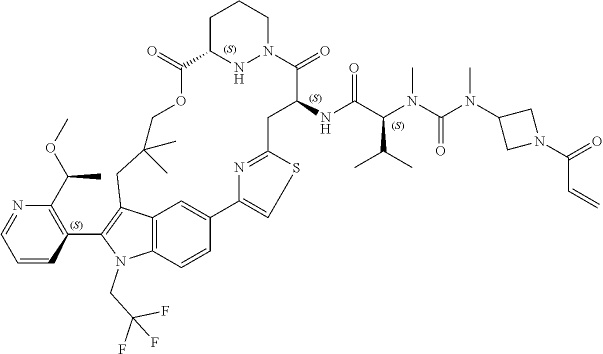 |
| A712 | 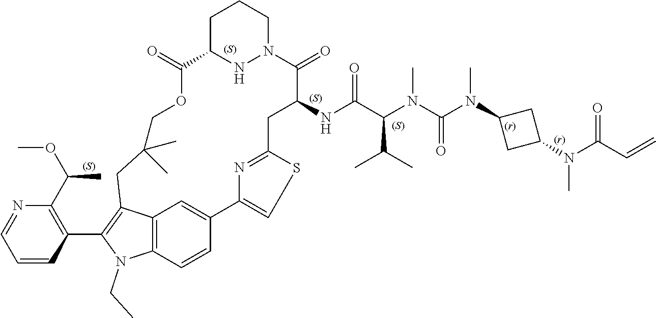 |
| A713 | 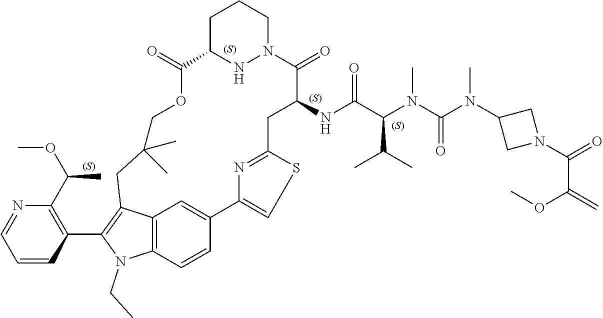 |
| A714 | 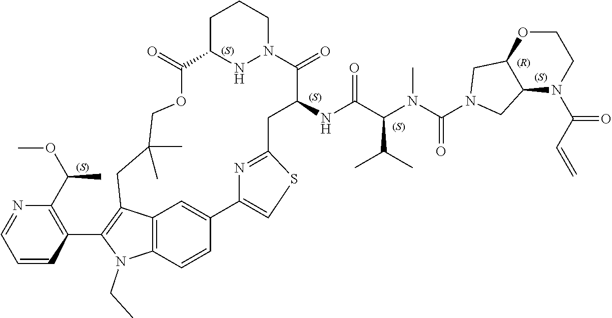 |
| A715 | 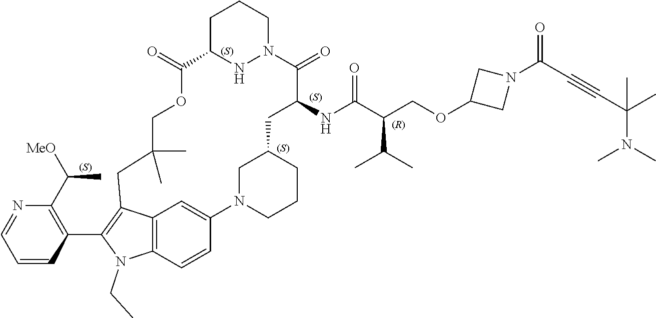 |
| A716 | 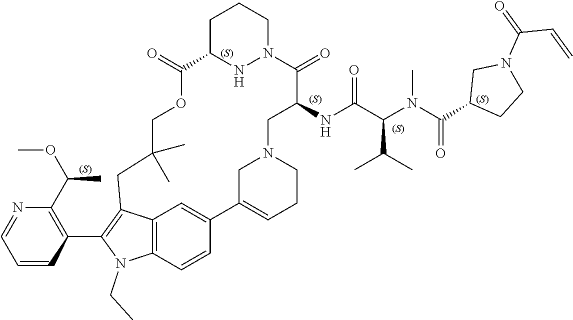 |
| A717 | 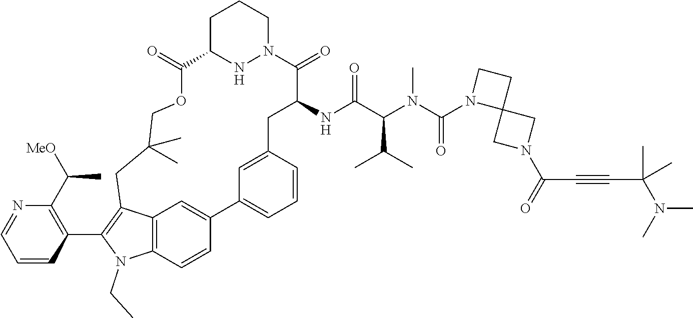 |
| A718 | 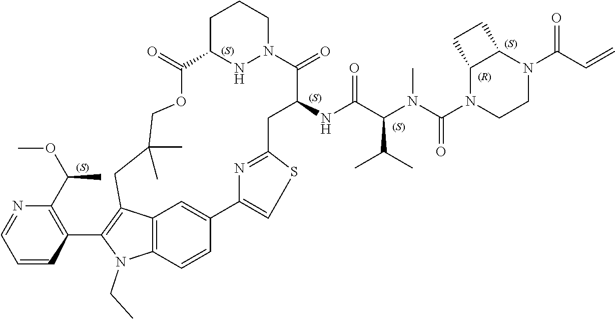 |
| A719 | 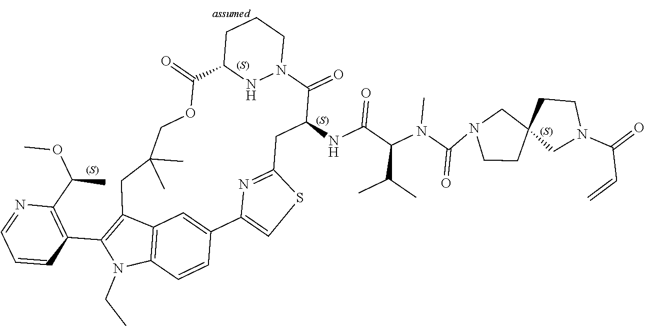 |
| A720 | 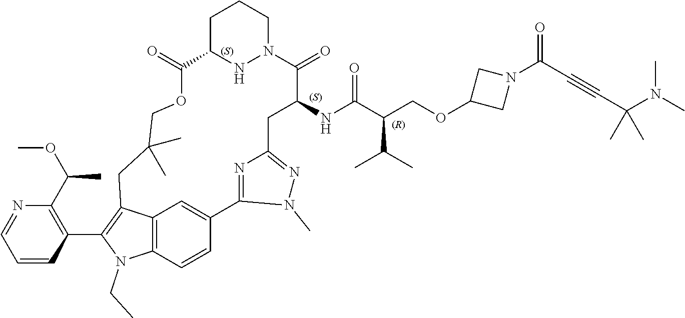 |
| A721 | 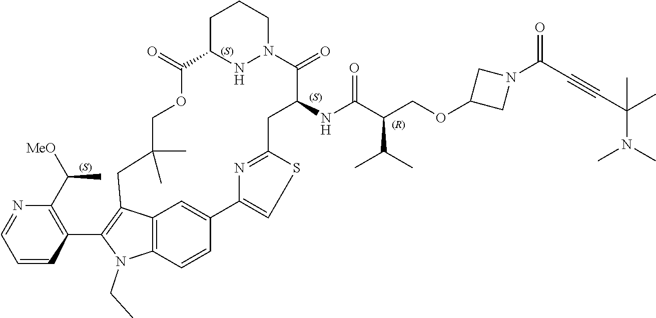 |
| A722 | 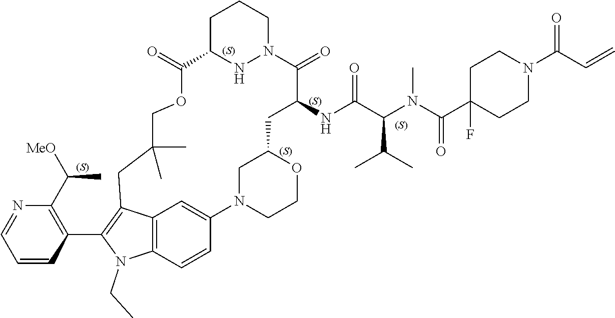 |
| A723 | 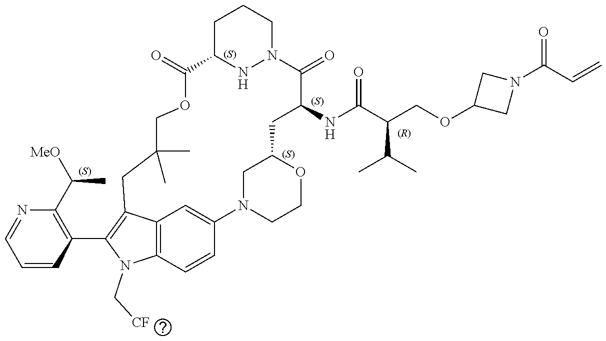 |
| A724 | 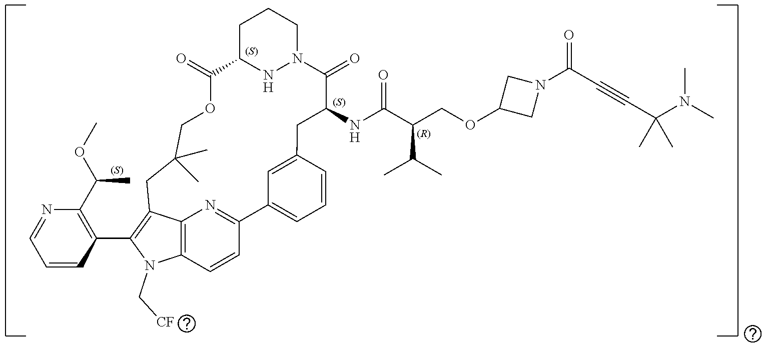 |
| A725 | 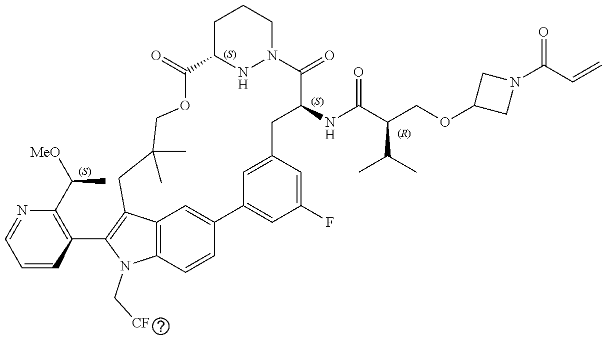 |
| A726 | 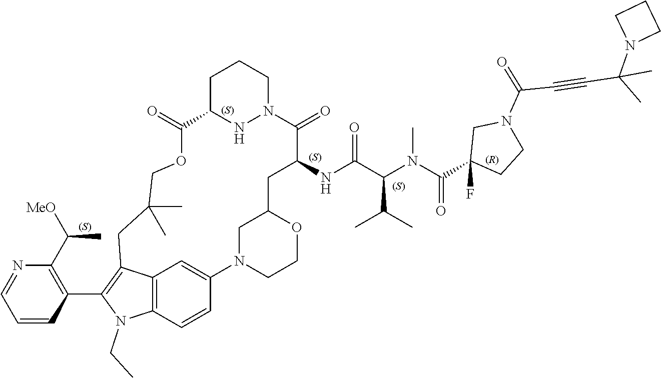 |
| A727 | 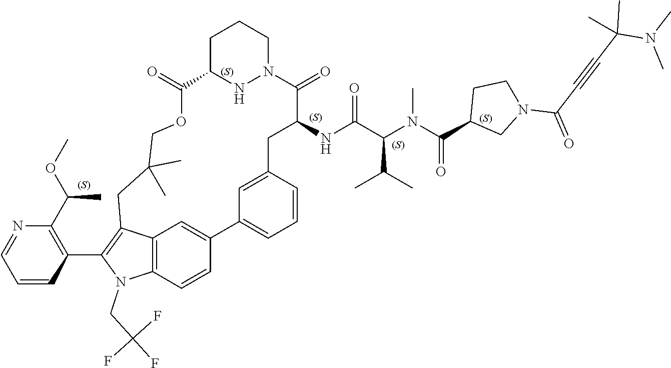 |
| A728 | 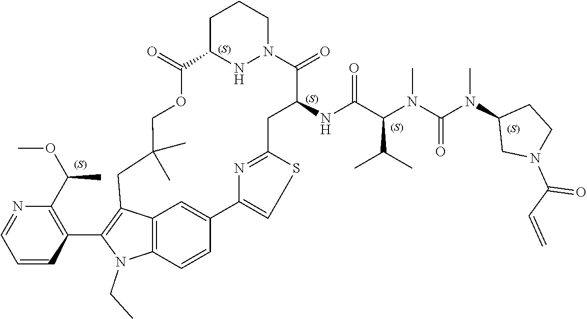 |
| A729 | 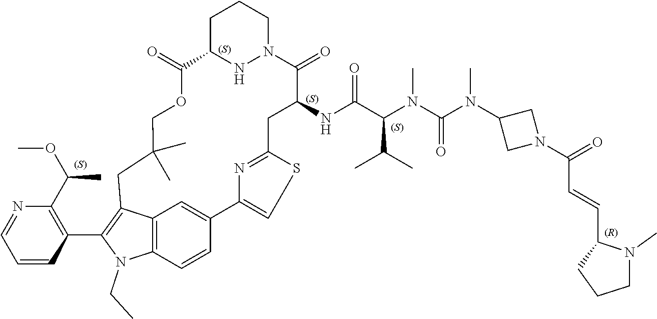 |
| A730 | 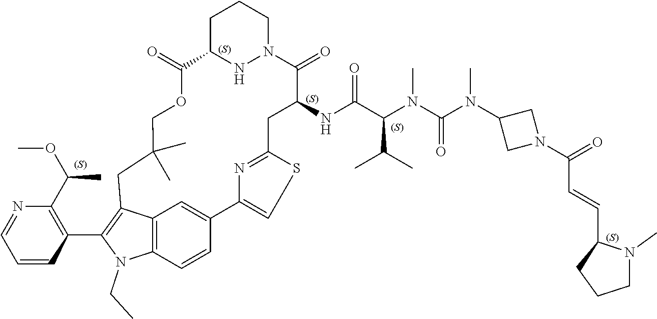 |
| A731 | 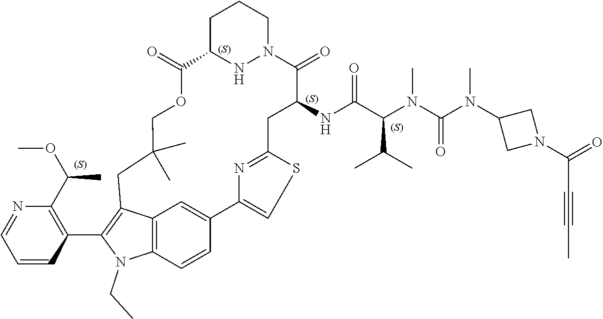 |
| A732 | 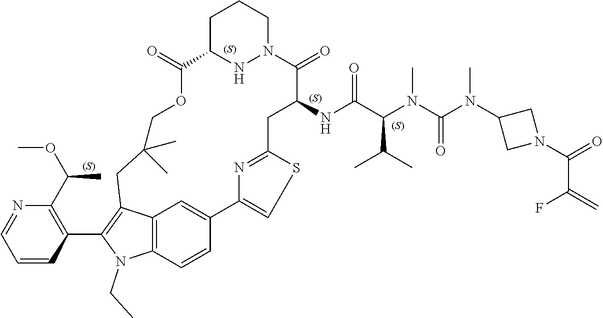 |
| A733 | 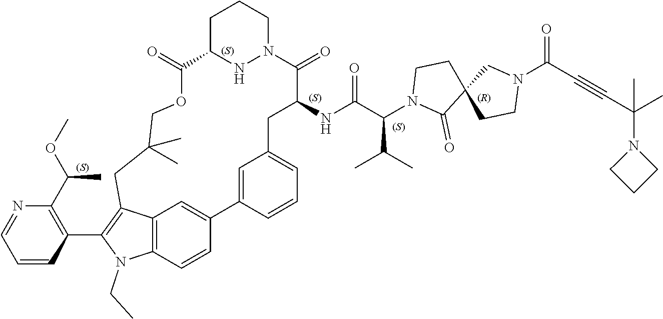 |
| A734 | 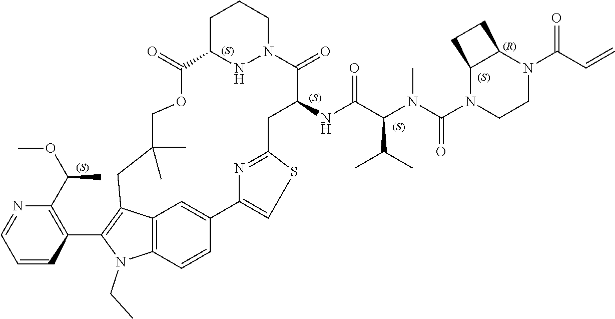 |
| A735 | 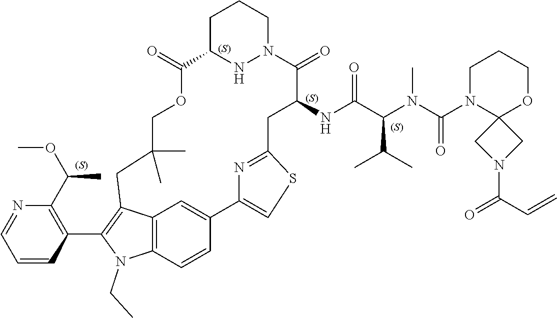 |
| A736 | 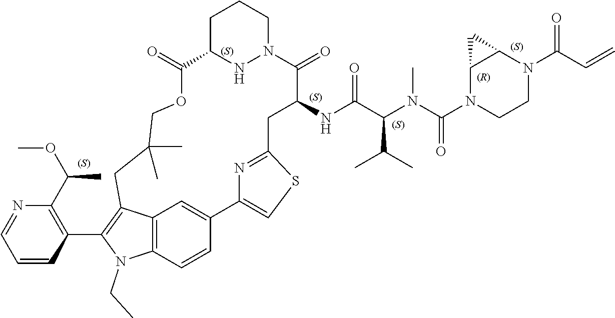 |
| A737 | 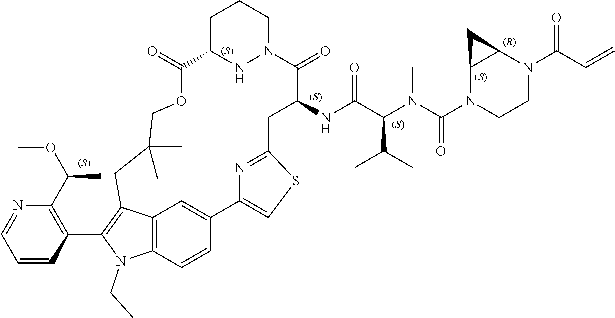 |
| A738 | 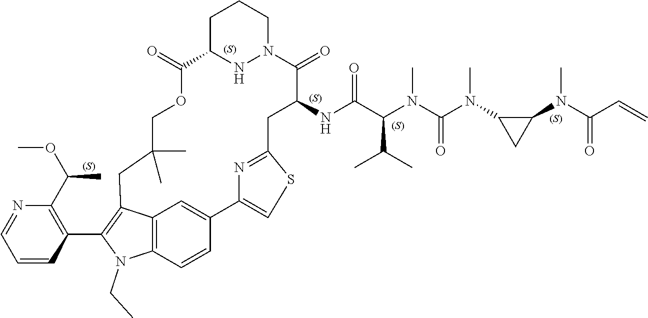 |
| A739 | 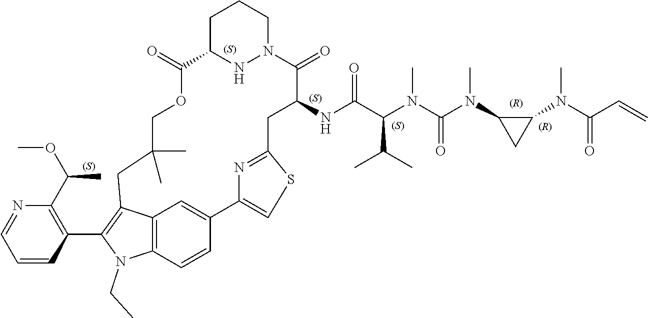 |
| A740 | 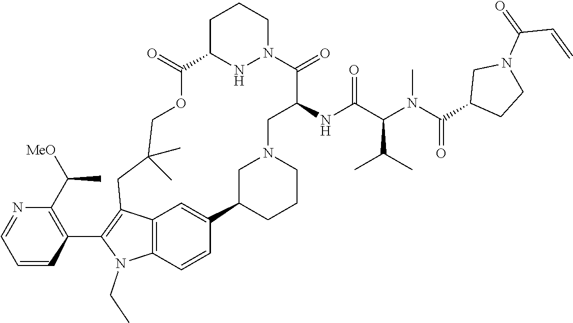 |
| A741 | 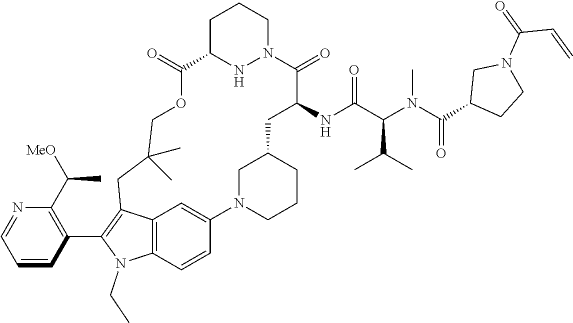 |

Note that some compounds are shown with bonds as flat or wedged. In some instances, the relative stereochemistry of steroisomers has been determined; in some instances, the abolute sterochemistry has been determined. In some instances, a single Example number corresponds to a mixture of steroisomers. All stereoisomers of the compounds of the foregoing table are contemplated by the present invention. In particular embodiments, an atropisomer of a compound of the foregoing table is contemplated.
Brackets are to be ignored.
*The activity of this stereoisomer may, in fact, be attributable to the presence of a small amount of the stereoisomer with the (S) configuration at the —NC(O)—CH(CH₃)₂—N(CH₃)— position.

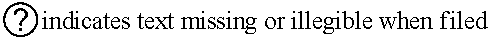

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B1 | 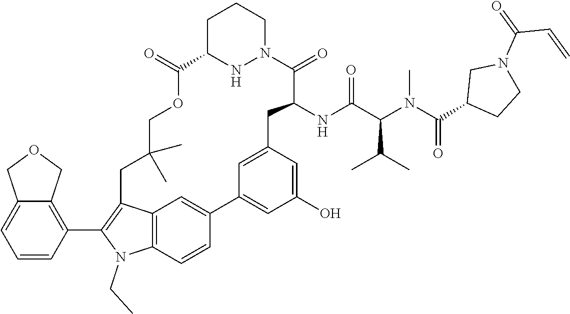 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| B2 | 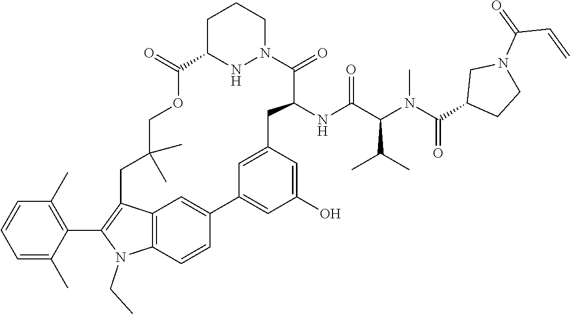 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| B3 | 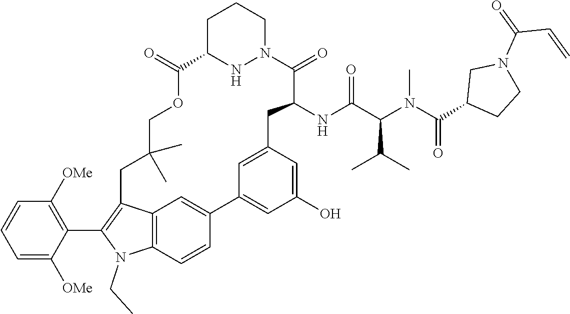 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| B4 | 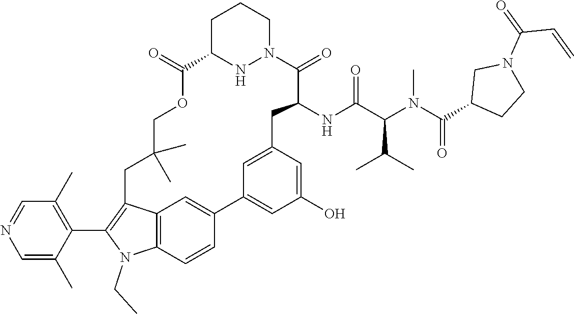 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| B5 | 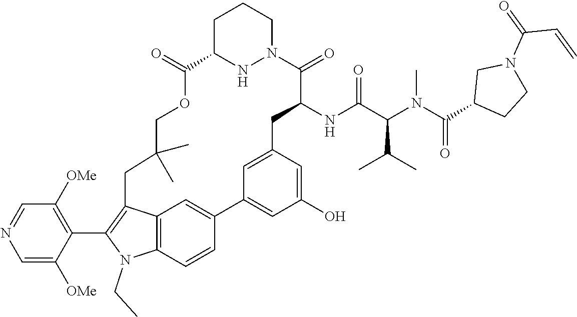 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| B6 | 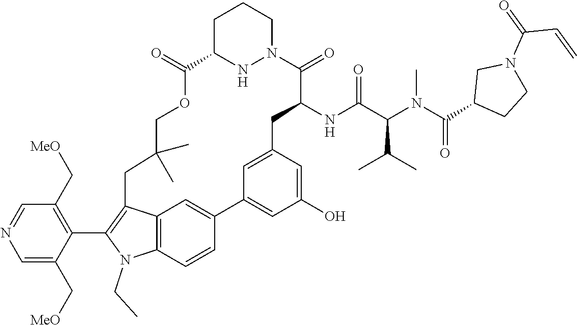 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| B7 | 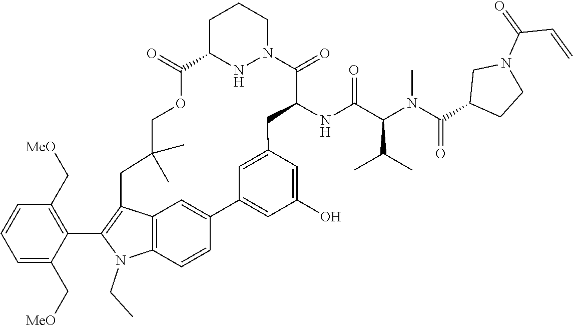 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| B11 | 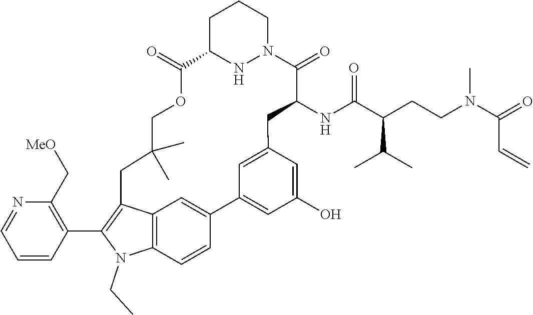 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| B12 | 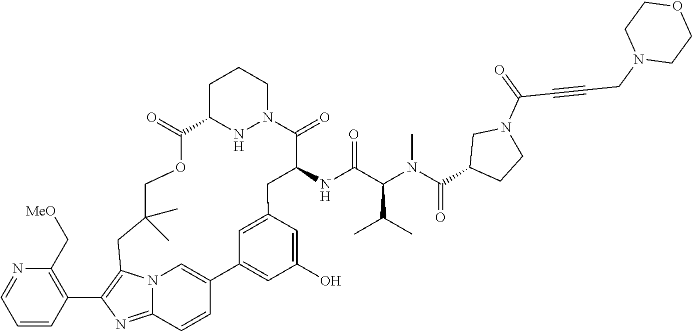 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| B13 | 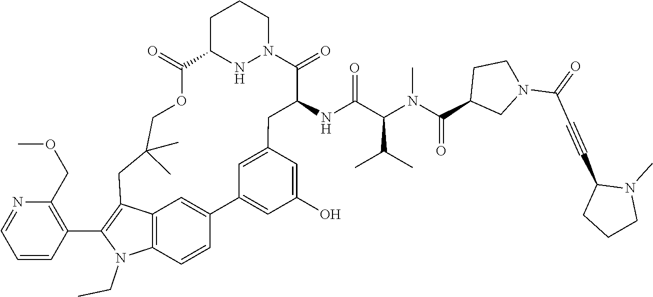 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| B18 | 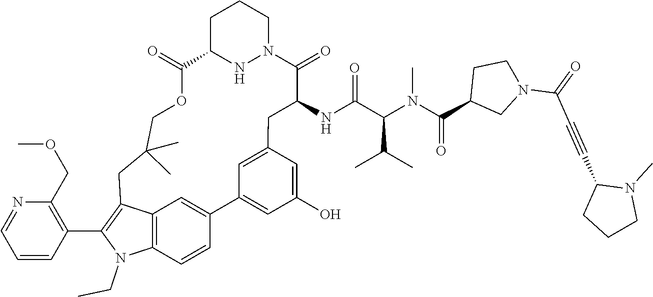 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| B21 | 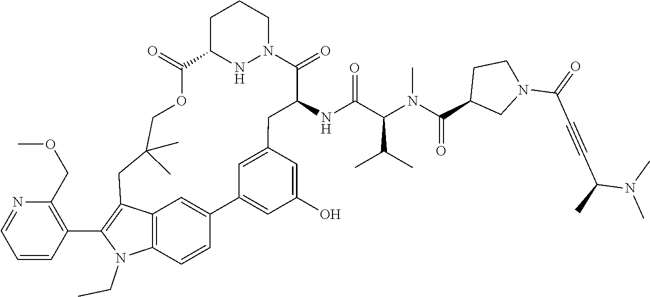 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)N1CCC[C@H](N1)C(=O)OCC(C)(C)C2] |
| B22 | 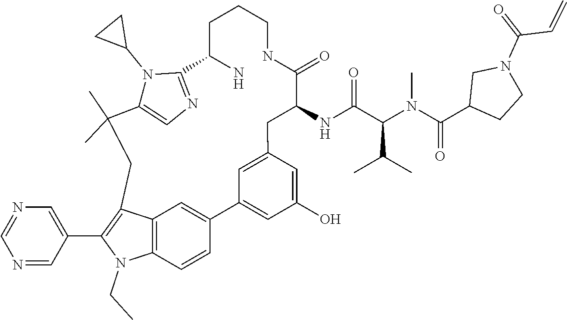 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)c3cnc(n3C3CC3)[C@@H](N)CCCNC2=O)C(C)C)C1] |
| B25 | 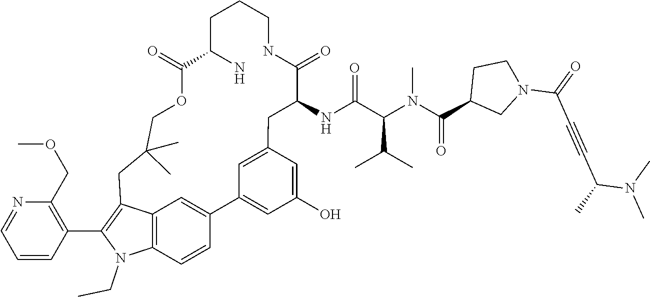 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)NCCC[C@H](N)C(=O)OCC(C)(C)C2] |
| B27 | 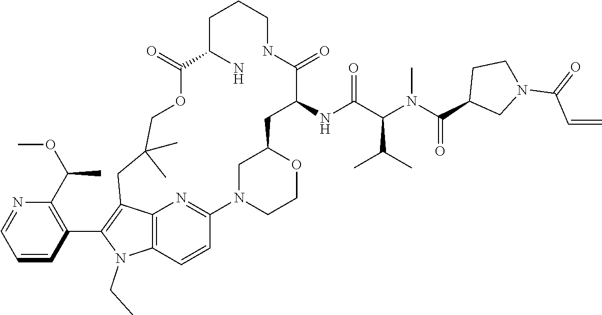 |
| B28 | 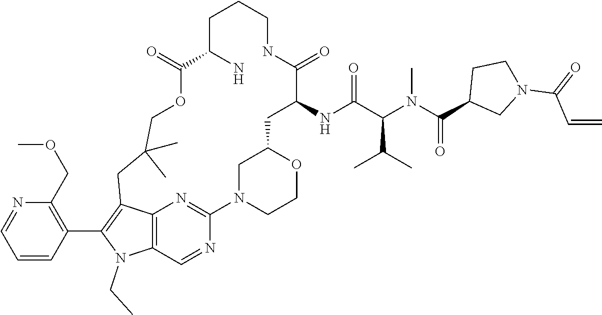 [SMILES: C=CC(=O)N1CC[C@H](C(=O)N(C)[C@H](C(=O)N[C@H]2C[C@H]3CN(CCO3)c3ncc4c(n3)c(c(-c3cccnc3COC)n4CC)CC(C)(C)COC(=O)[C@@H](N)CCCNC2=O)C(C)C)C1] |
| B29 | 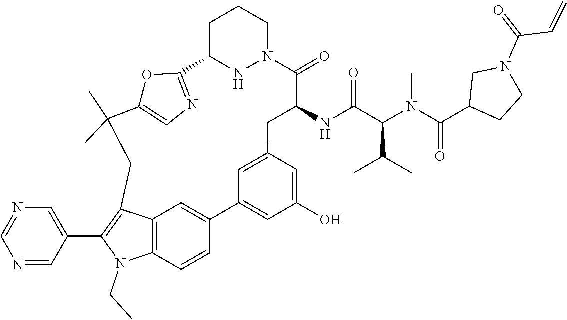 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| B30 | 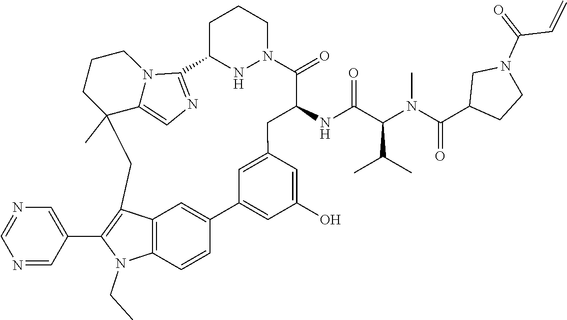 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| B32 | 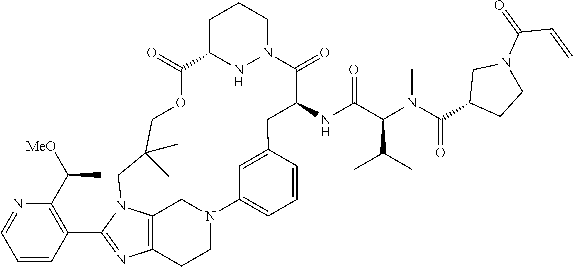 [SMILES: C=CC(=O)N1CC[C@H](C(=O)N(C)[C@H](C(=O)N[C@H]2Cc3cccc(c3)N3CCc4nc(-c5cccnc5[C@H](C)OC)n(c4C3)CC(C)(C)COC(=O)[C@@H]3CCCN(N3)C2=O)C(C)C)C1] |
| B34 | 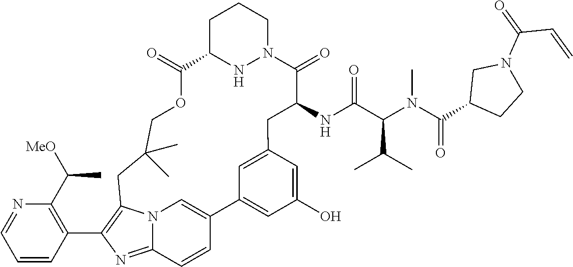 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| B38 | 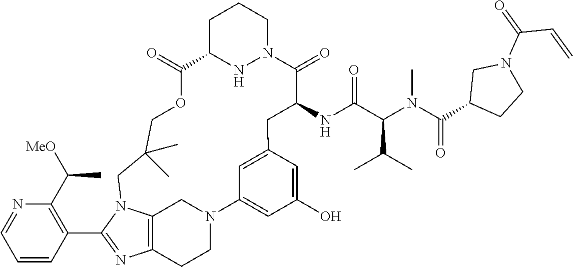 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| B47 | 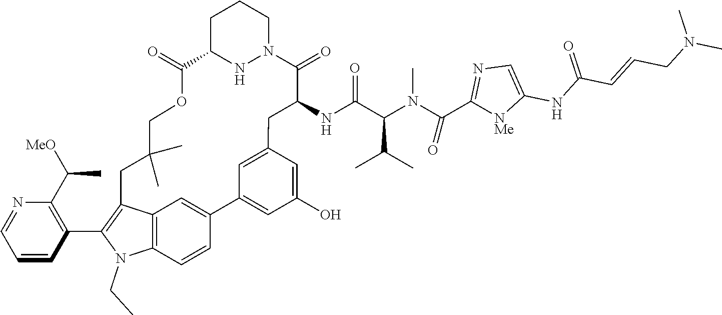 |
| B64 | 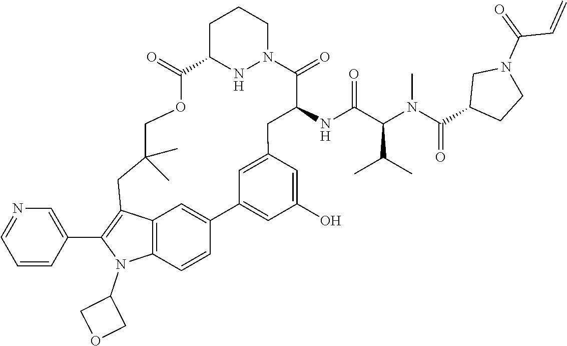 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| B65 | 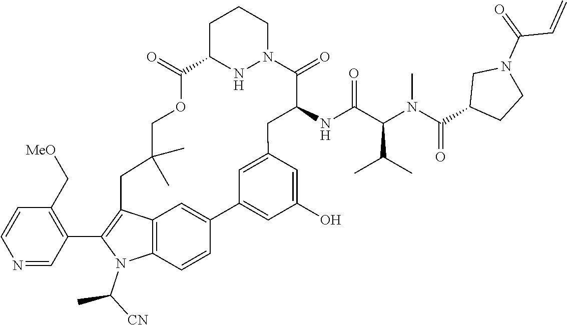 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| B66 | 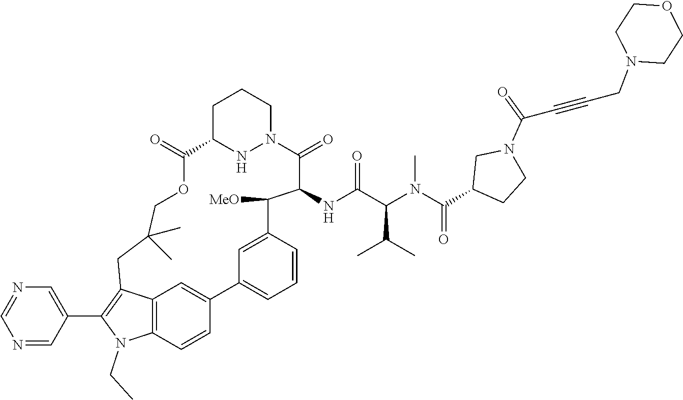 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B70 | 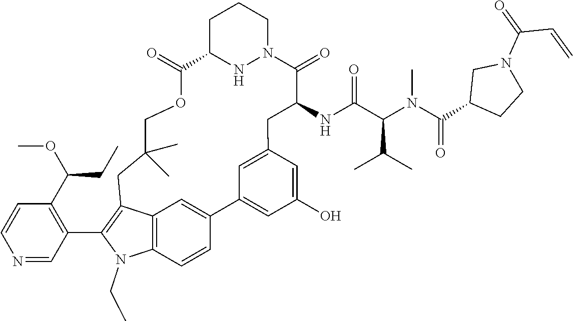 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](CC)OC)n4CC)CC(C)(C)COC(=O)[C@@H]3CCCN(N3)C2=O)C(C)C)C1] |
| B73 | 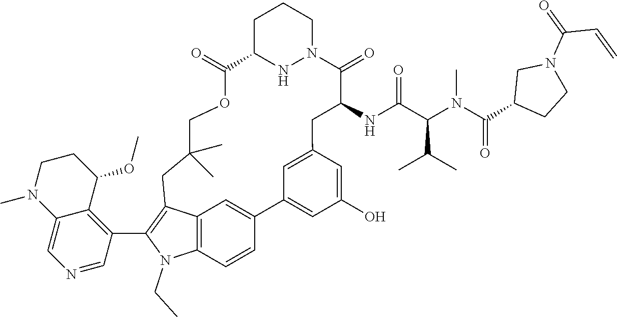 [SMILES: C=CC(=O)N1CC[C@H](C(=O)N(C)[C@H](C(=O)N[C@H]2Cc3cc(O)cc(c3)-c3ccc4c(c3)c(c(-c3cncc5c3[C@@H](OC)CCN5C)n4CC)CC(C)(C)COC(=O)[C@@H]3CCCN(N3)C2=O)C(C)C)C1] |
| B74 | 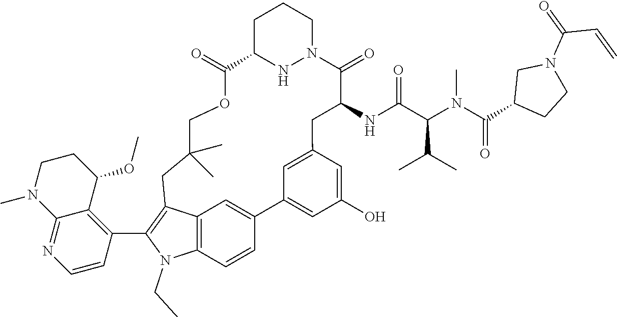 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| B75 | 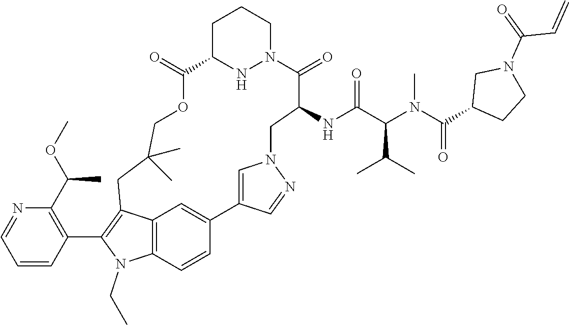 [SMILES: C=CC(=O)N1CC[C@H](C(=O)N(C)[C@H](C(=O)N[C@H]2Cn3cc(cn3)-c3ccc4c(c3)c(c(-c3cccnc3[C@H](C)OC)n4CC)CC(C)(C)COC(=O)[C@@H]3CCCN(N3)C2=O)C(C)C)C1] |
| B76 | 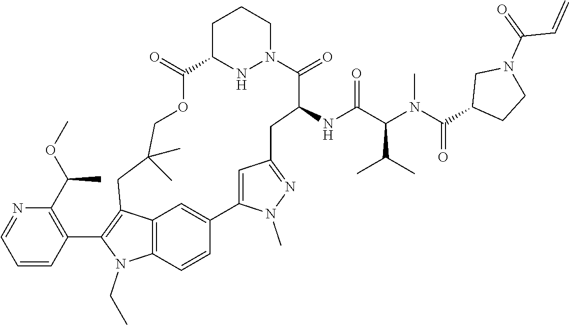 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| B77 | 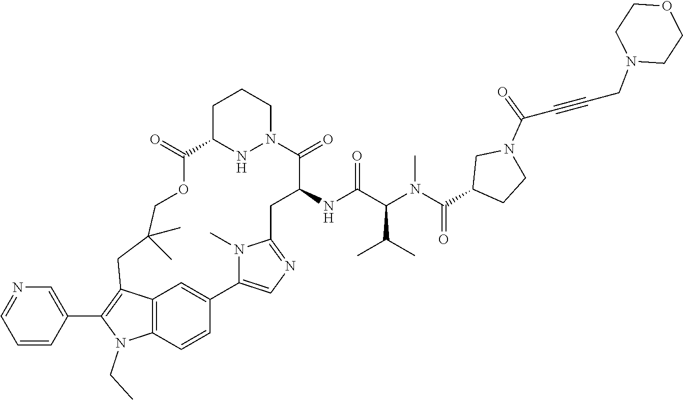 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B81 | 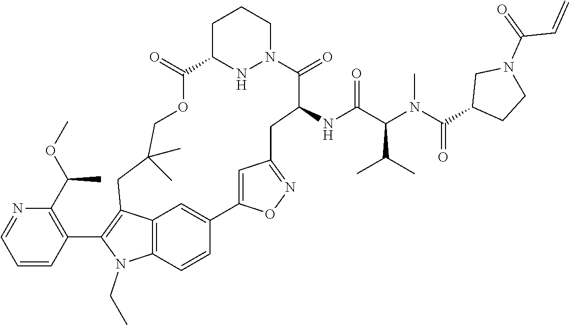 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| B83 | 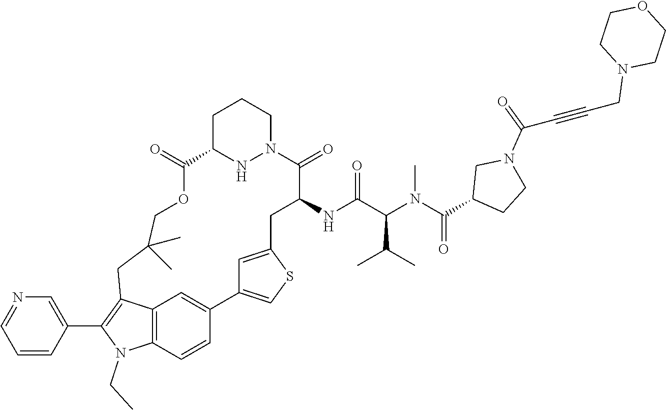 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B85 | 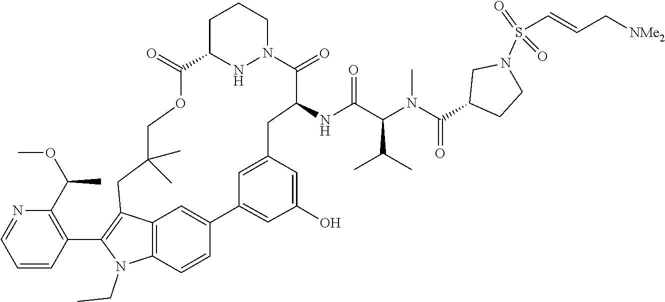 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| B86 | 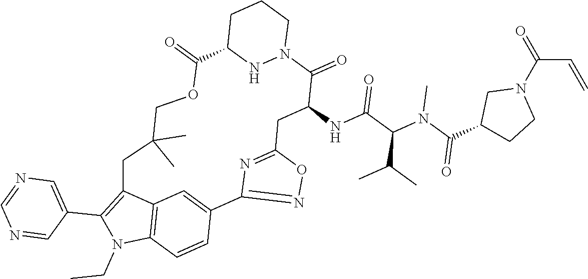 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| B87 | 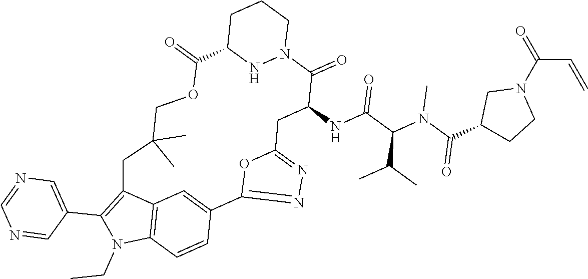 [SMILES: C=CC(=O)N1CC[C@H](C(=O)N(C)[C@H](C(=O)N[C@H]2Cc3nnc(o3)-c3ccc4c(c3)c(c(-c3cncnc3)n4CC)CC(C)(C)COC(=O)[C@@H]3CCCN(N3)C2=O)C(C)C)C1] |
| B88 | 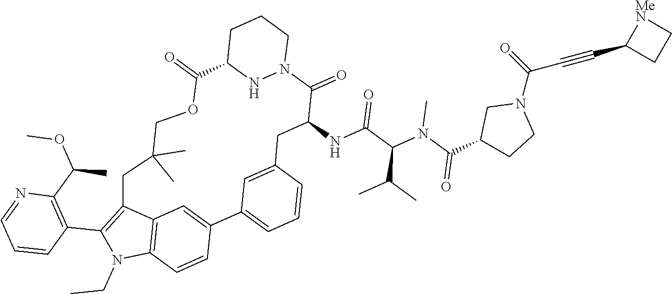 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| B89 | 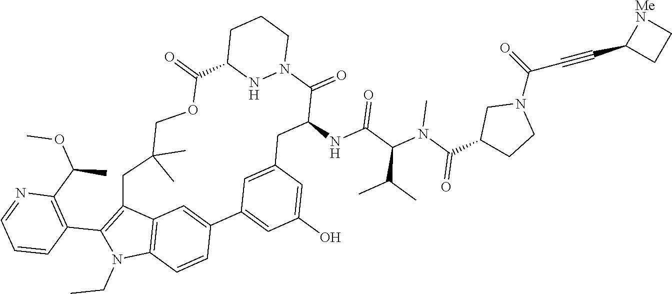 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| B90 | 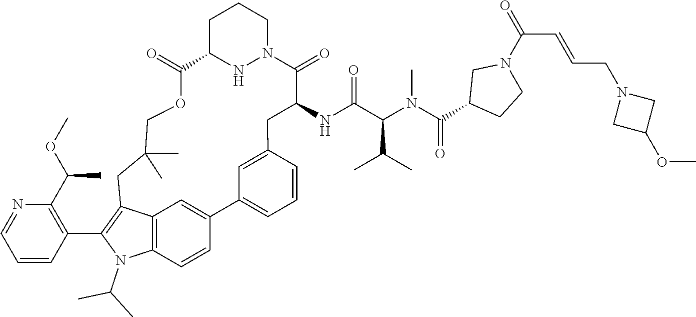 [SMILES: COC1CN(C/C=C/C(=O)N2CC[C@H](C(=O)N(C)[C@H](C(=O)N[C@H]3Cc4cccc(c4)-c4ccc5c(c4)c(c(-c4cccnc4[C@H](C)OC)n5C(C)C)CC(C)(C)COC(=O)[C@@H]4CCCN(N4)C3=O)C(C)C)C2)C1] |
| B91 | 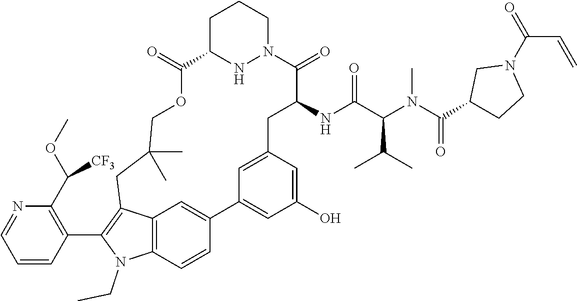 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| B96 | 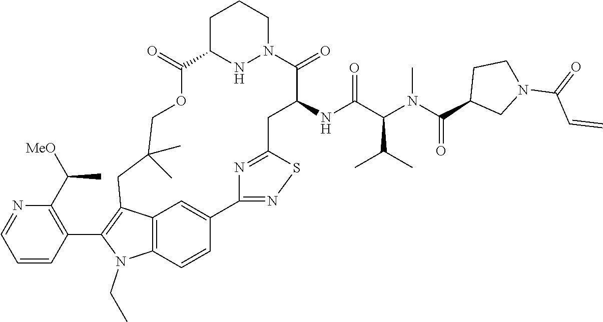 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| B97 | 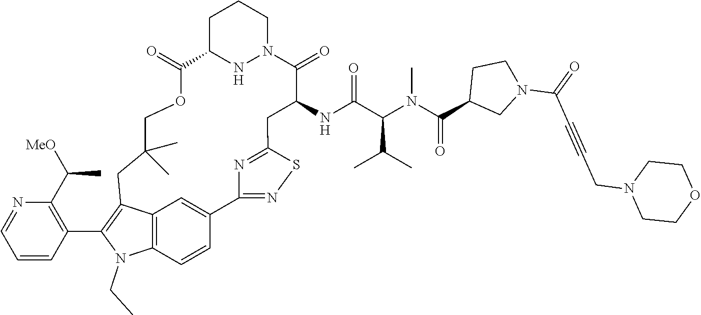 [SMILES: CCn1c(-c2cccnc2[C@H](C)OC)c2c3cc(ccc31)-c1nsc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B102 | 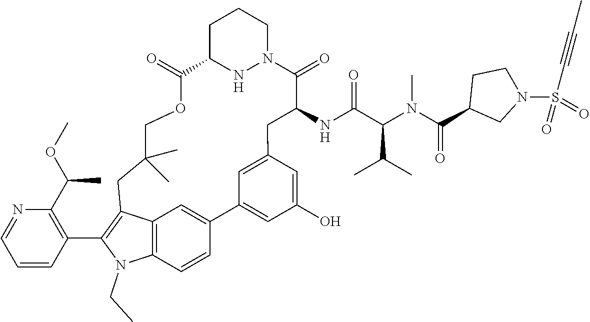 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| B103 | 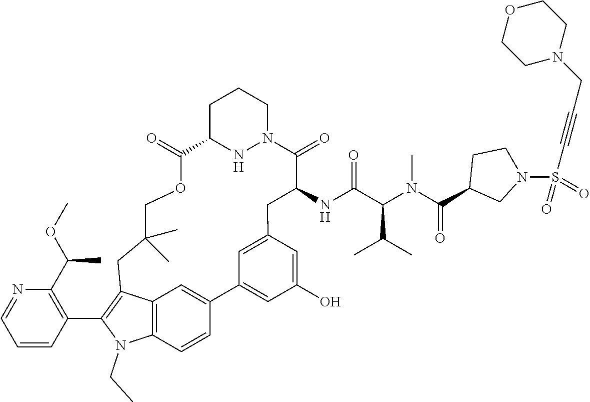 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| B104 | 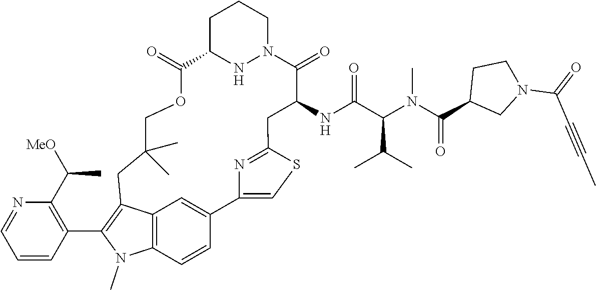 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| B106 | 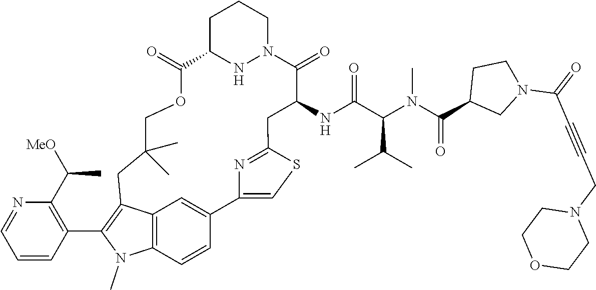 [SMILES: CO[C@@H](C)c1ncccc1-c1c2c3cc(ccc3n1C)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| B107 | 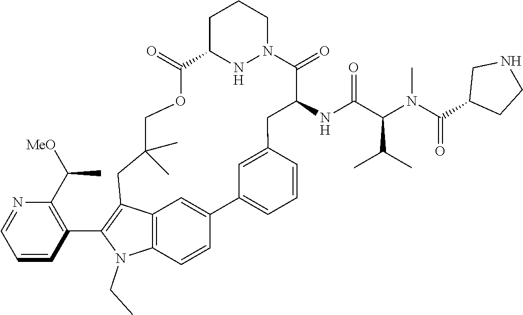 |
| B109 | 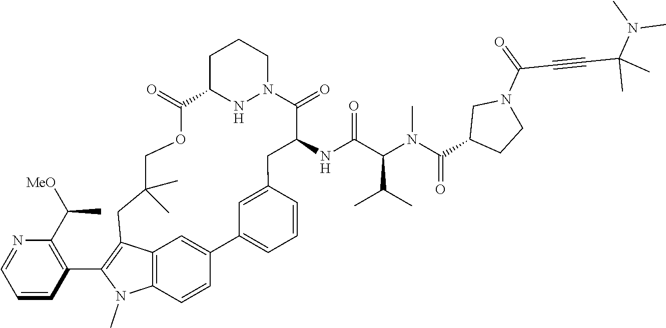 |
| B111 | 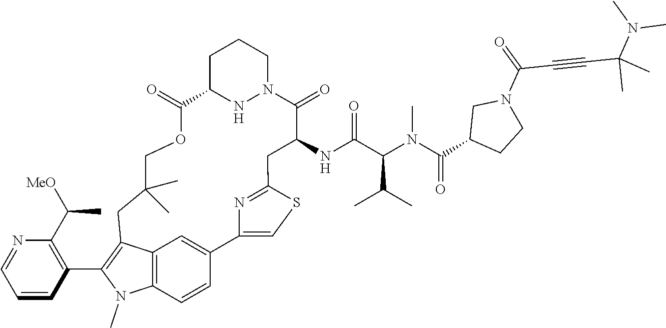 |
| B112 | 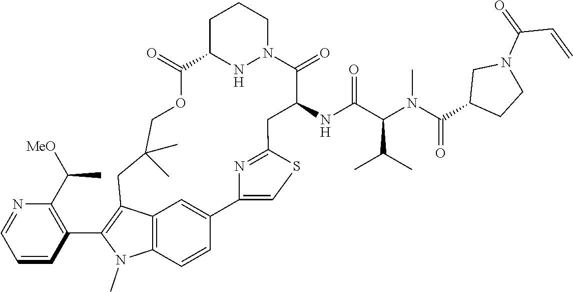 |
| B113 | 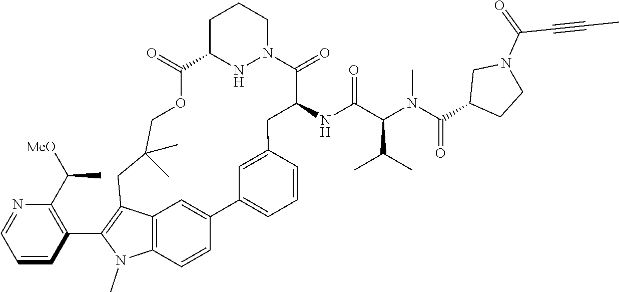 |
| B115 | 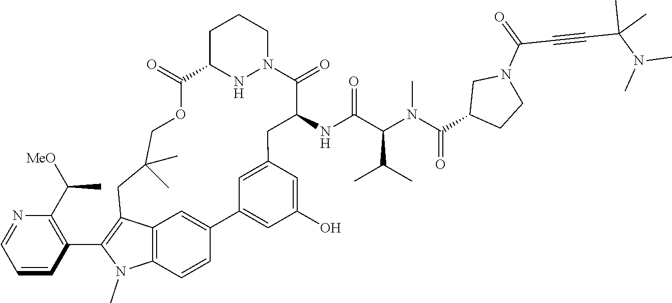 |
| B116 | 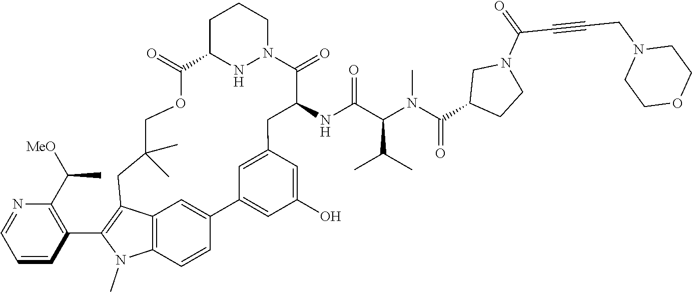 |
| B117 | 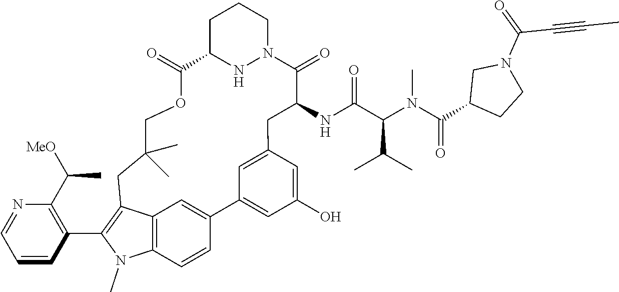 |
| B118 | 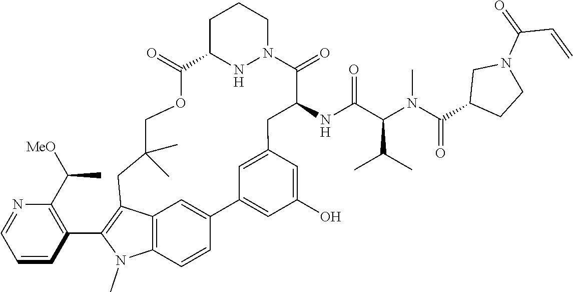 |
| B119 | 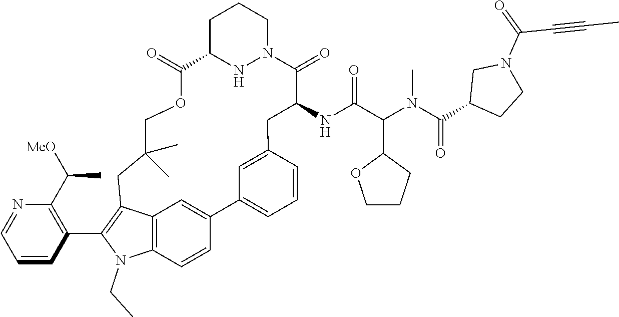 |
| B120 | 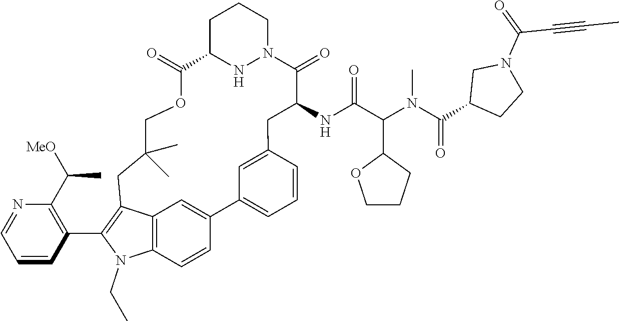 |
| B121 | 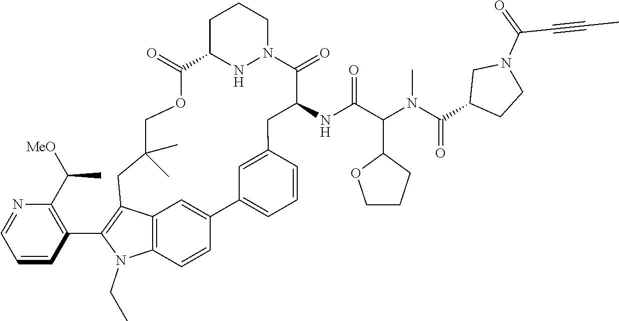 |
| B122 | 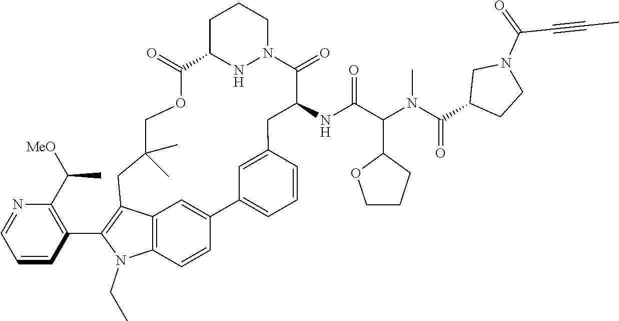 |
| B123 | 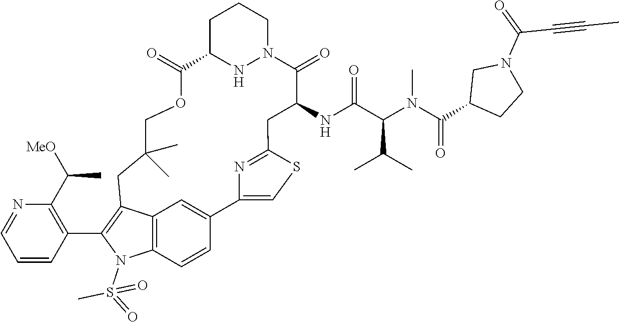 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| B124 | 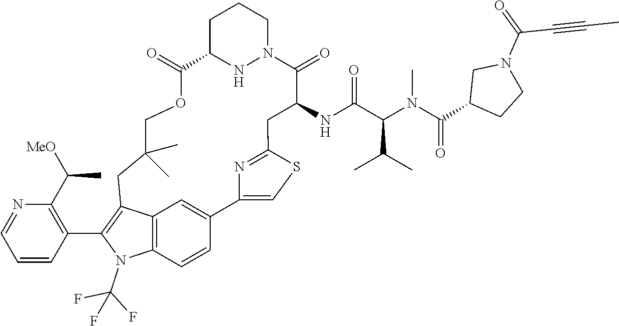 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| B126 | 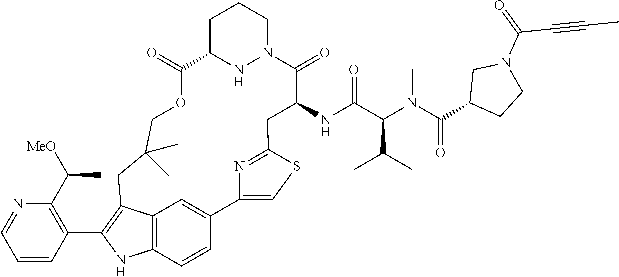 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| B127 | 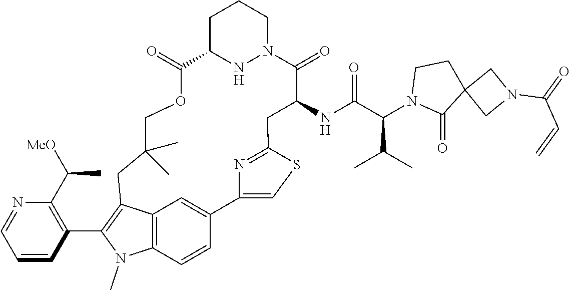 |
| B128 | 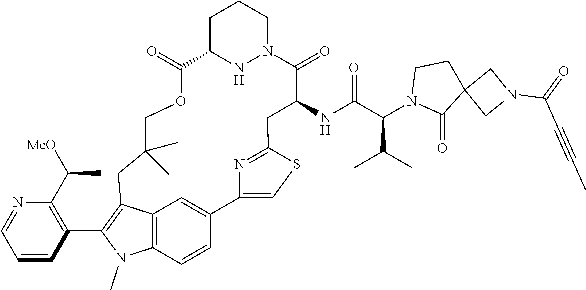 |
| B129 | 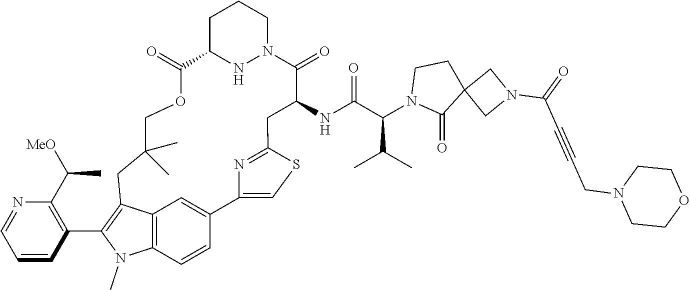 |
| B130 | 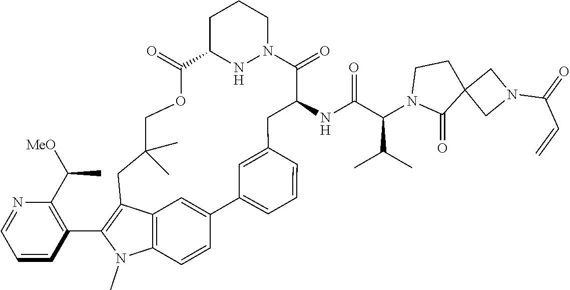 |
| B131 | 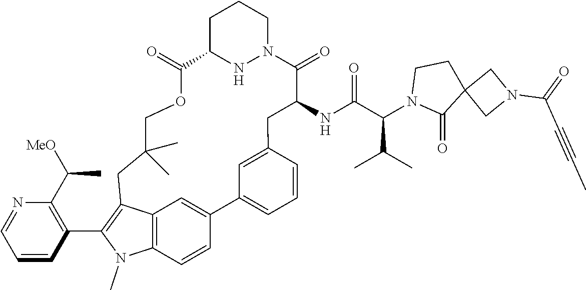 |
| B132 | 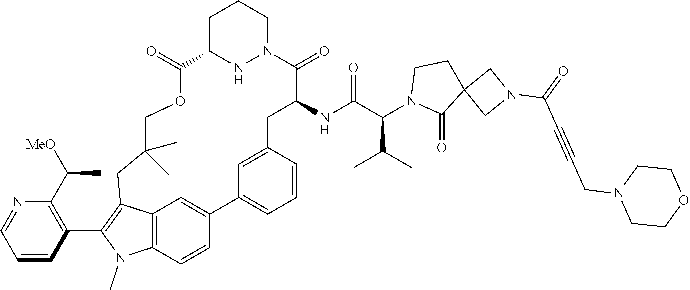 |
| B139 | 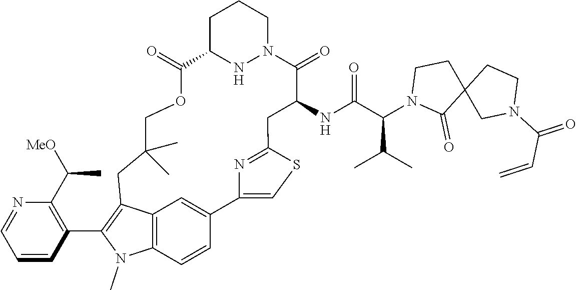 |
| B140 | 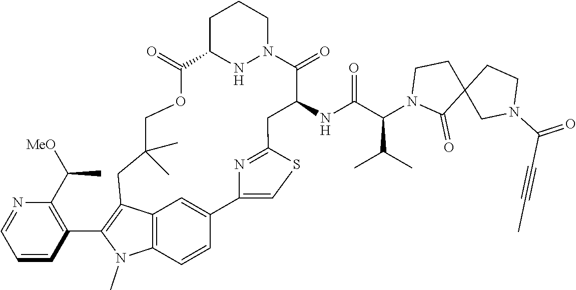 |
| B141 | 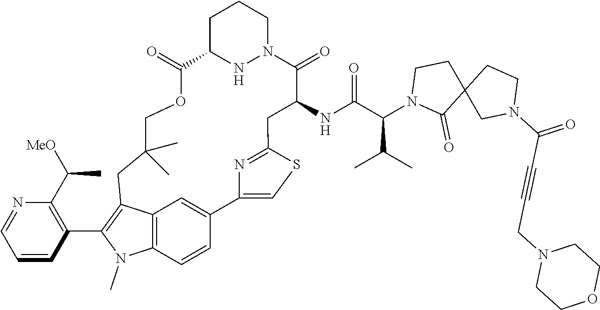 |
| B142 | 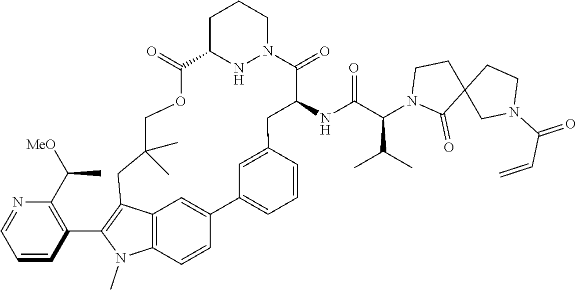 |
| B143 | 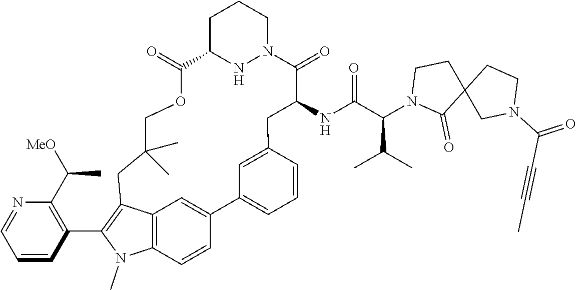 |
| B144 | 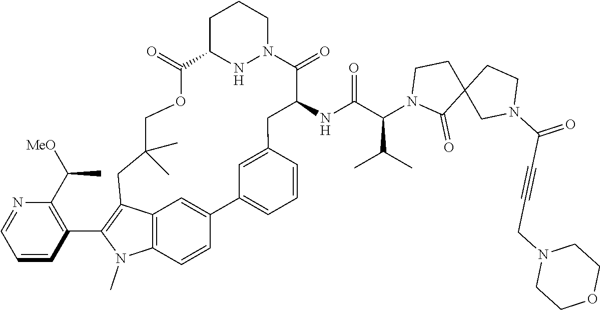 |
| B145 | 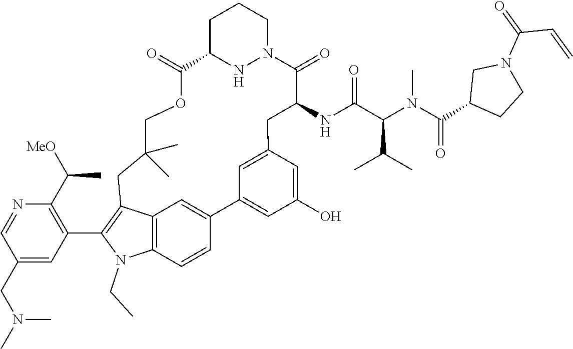 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B146 | 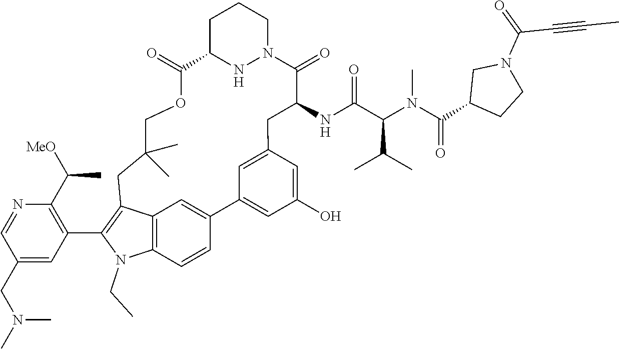 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B147 | 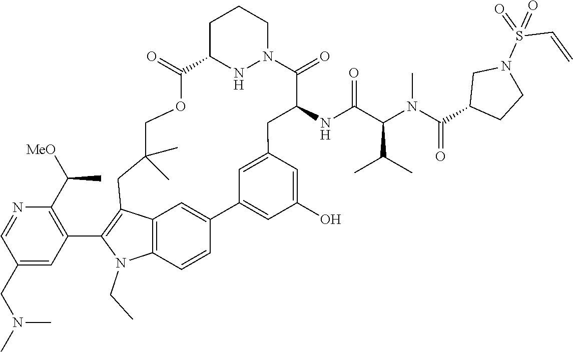 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| B148 | 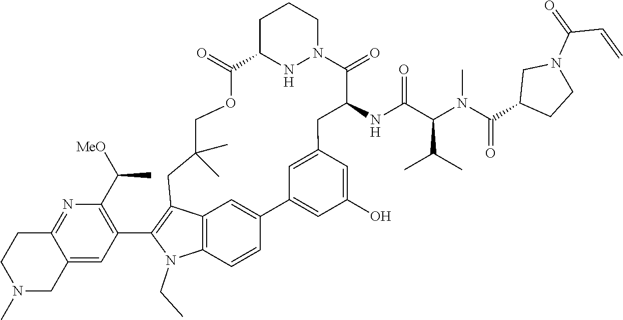 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| B149 | 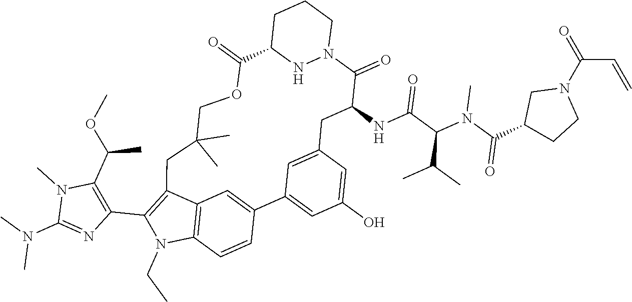 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| B150 | 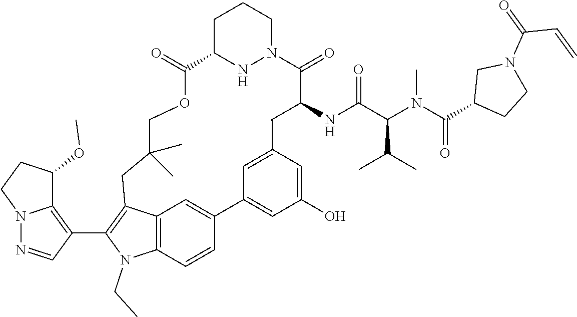 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| B161 | 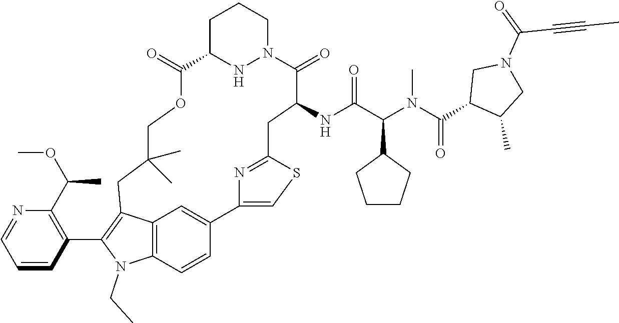 |
| B162 | 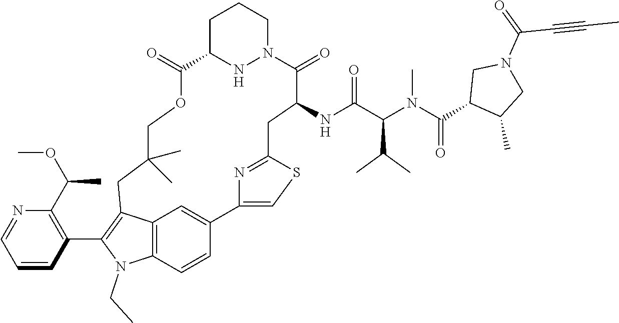 |
| B163 | 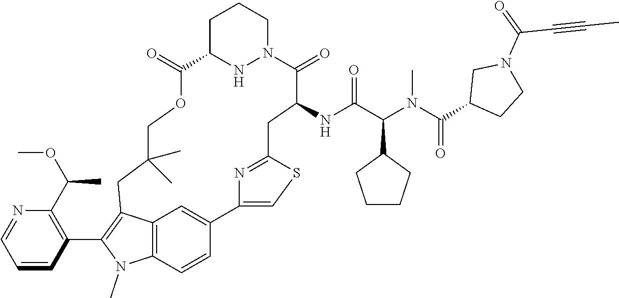 |
| B164 | 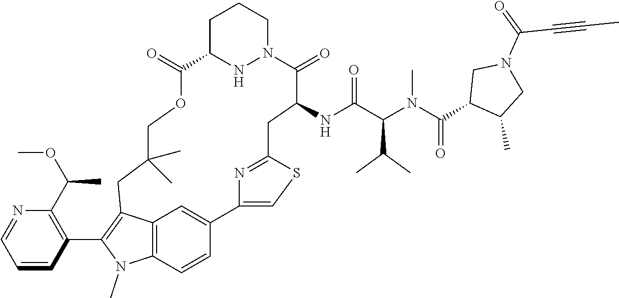 |
| B165 | 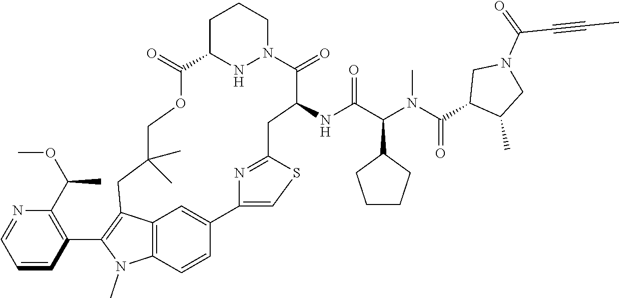 |
| B167 | 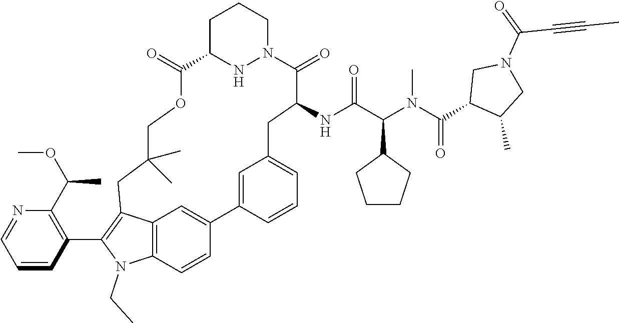 |
| B168 | 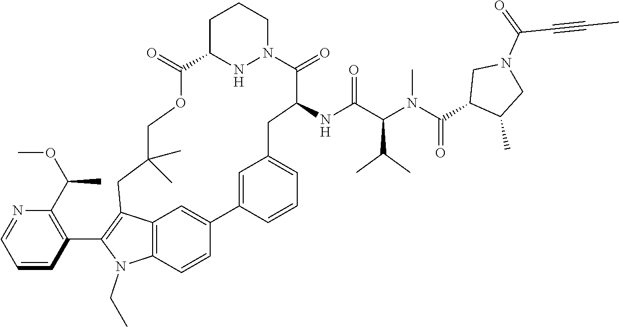 |
| B169 | 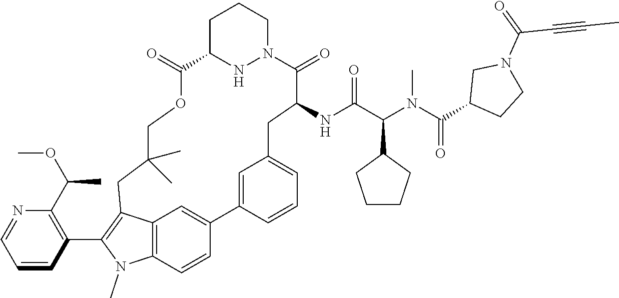 |
| B170 | 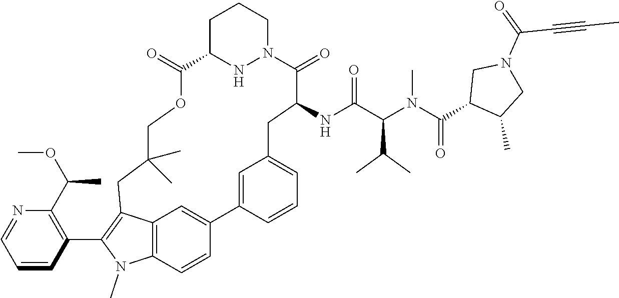 |
| B171 | 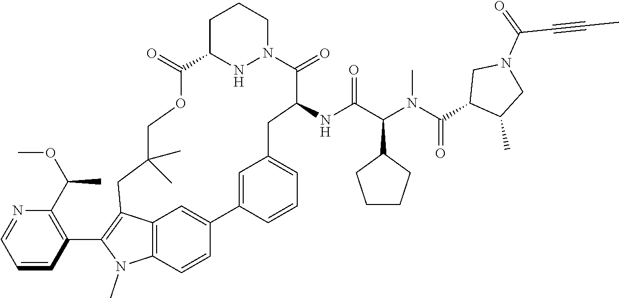 |
| B172 | 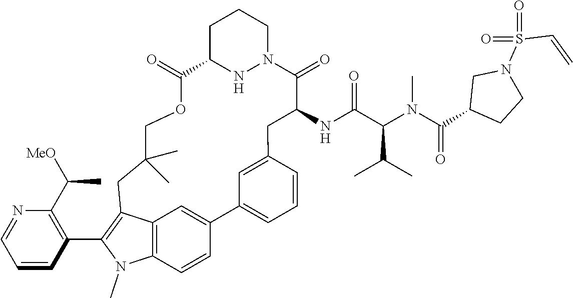 |
| B173 | 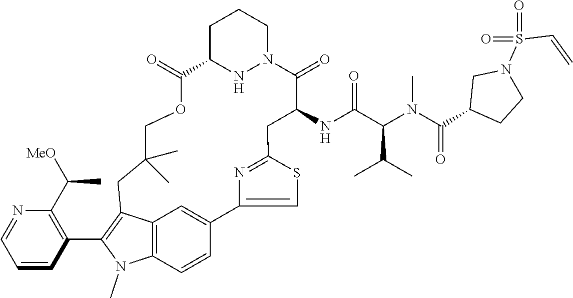 |
| B174 | 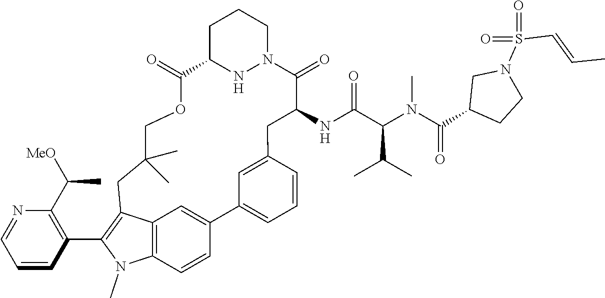 |
| B175 | 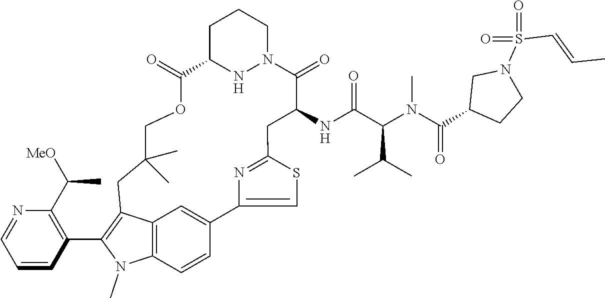 |
| B176 | 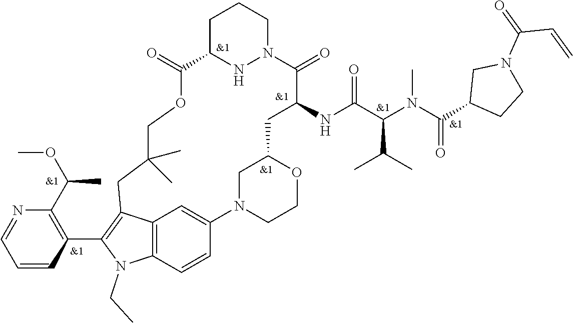 |
| B177 | 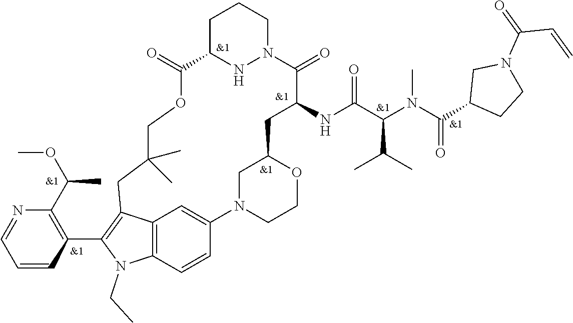 |
| B178 | 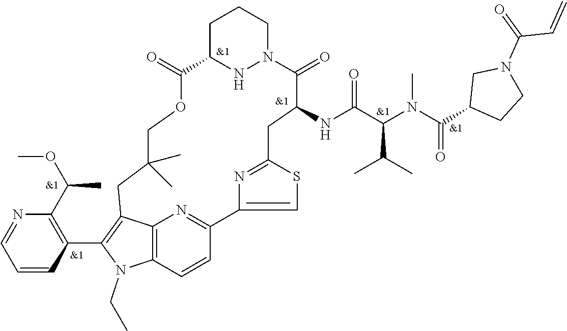 |
| B179 | 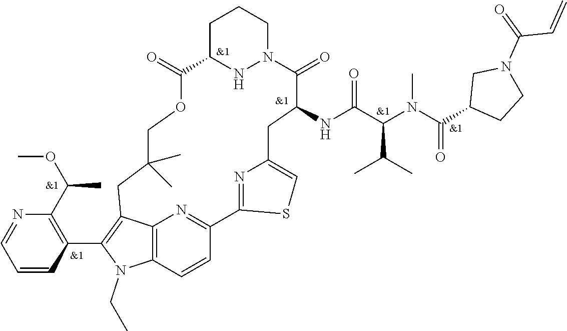 |
| B180 | 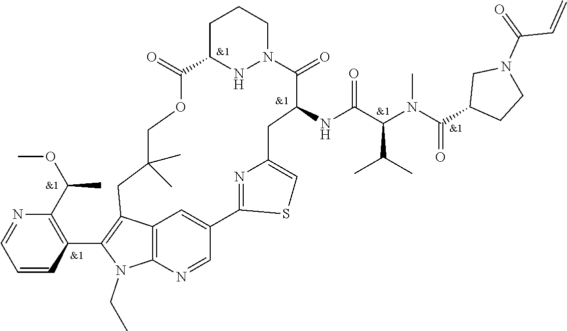 |
| B181 | 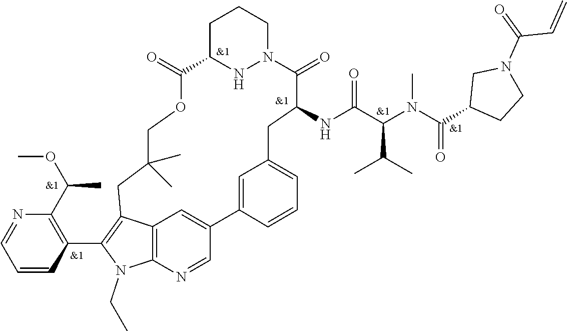 |
| B182 | 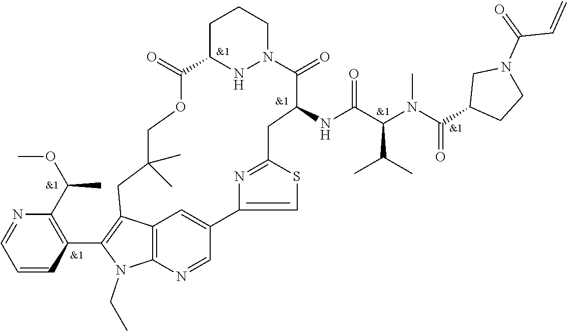 |
| B183 | 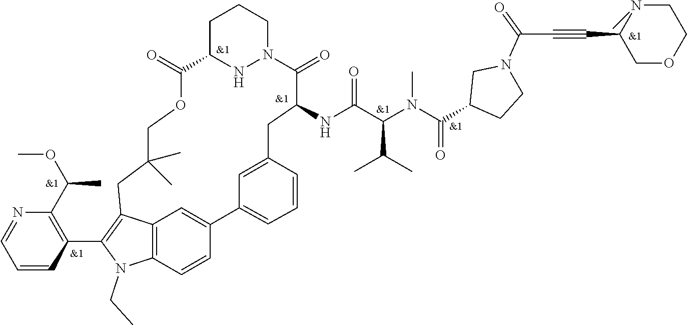 |
| B184 | 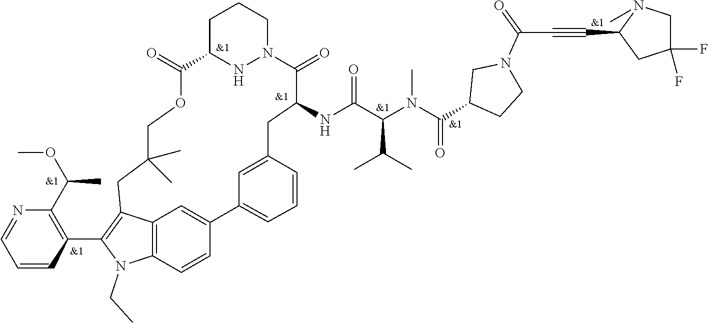 |
| B185 | 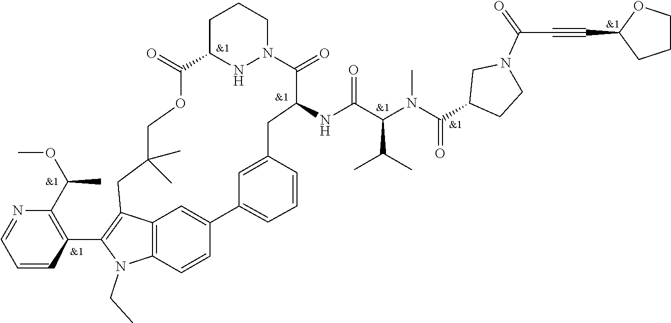 |
| B186 | 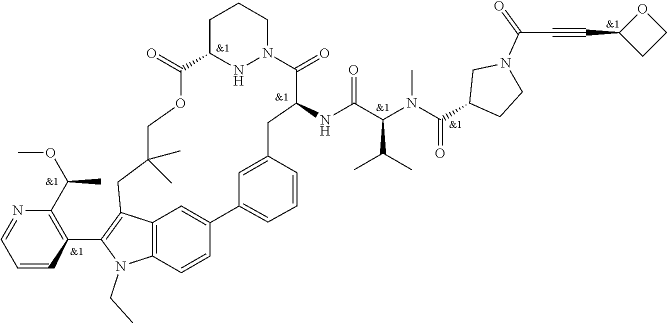 |
| B187 | 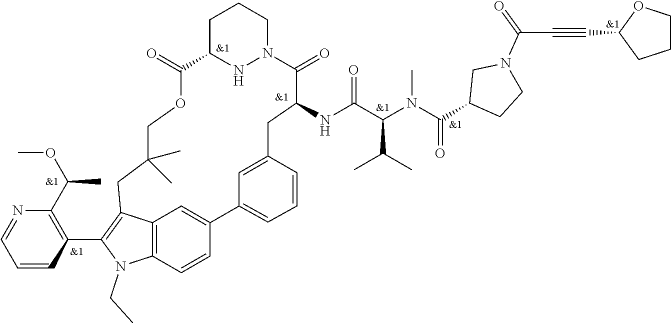 |
| B188 | 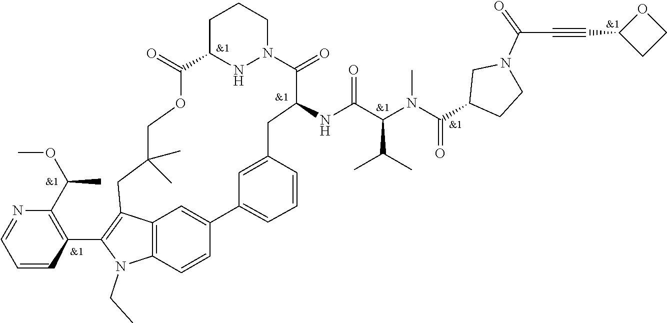 |
| B189 | 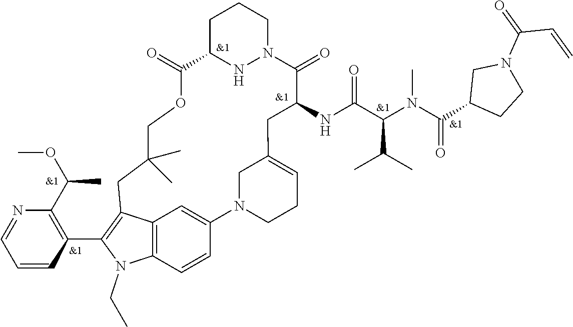 |
| B190 | 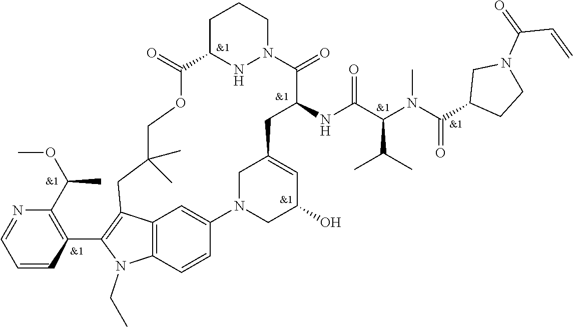 |
| B191 | 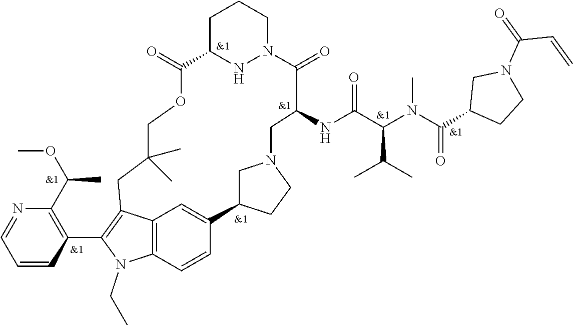 |
| B192 | 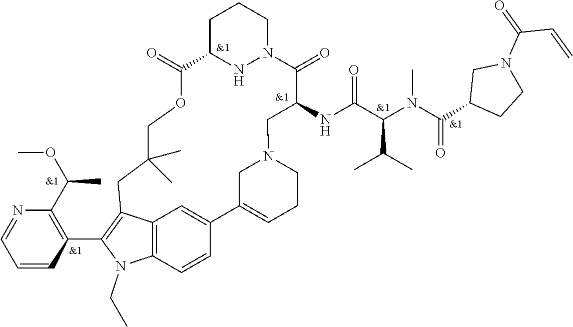 |
| B194 | 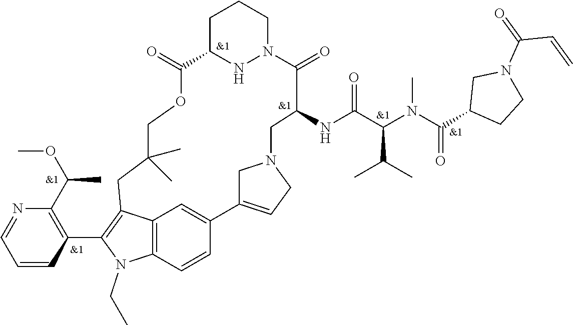 |
| B195 | 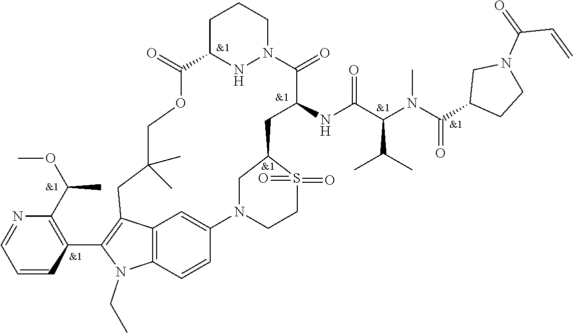 |
| B196 | 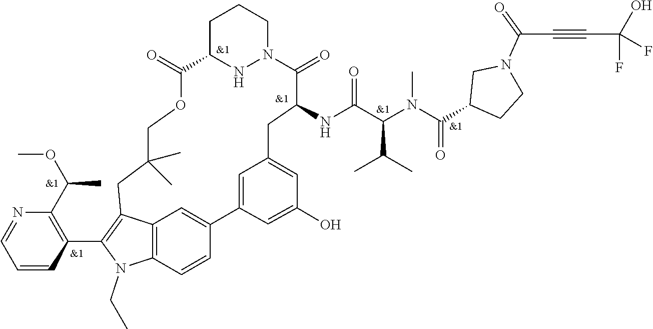 |
| B197 | 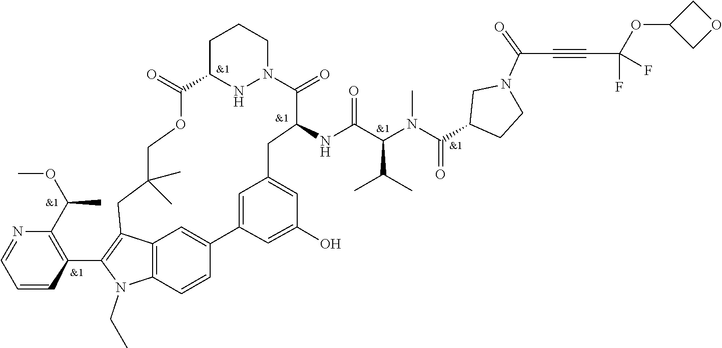 |
| B198 | 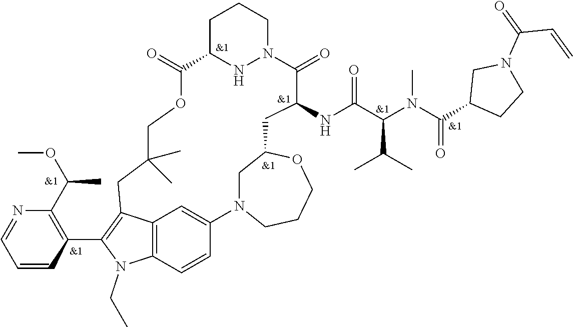 |
| B199 | 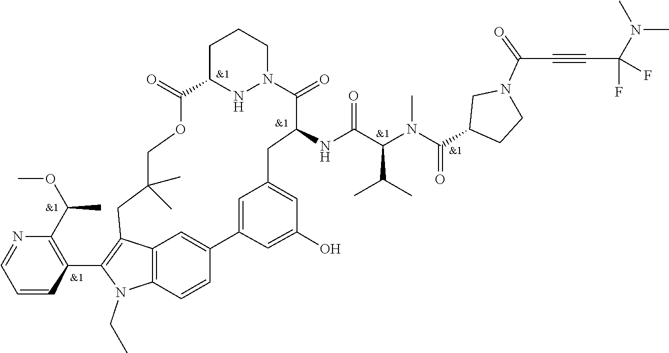 |
| B200 | 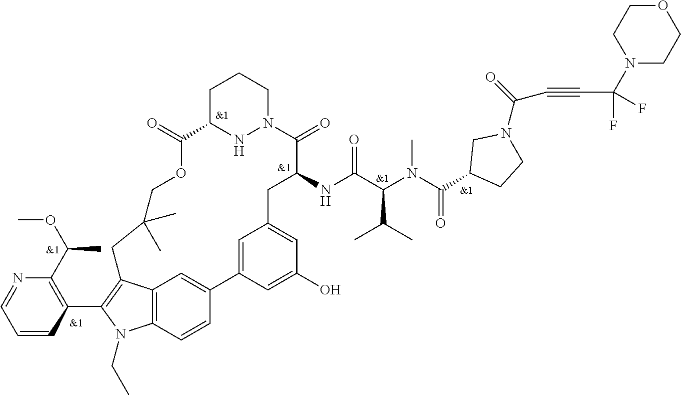 |
| B201 | 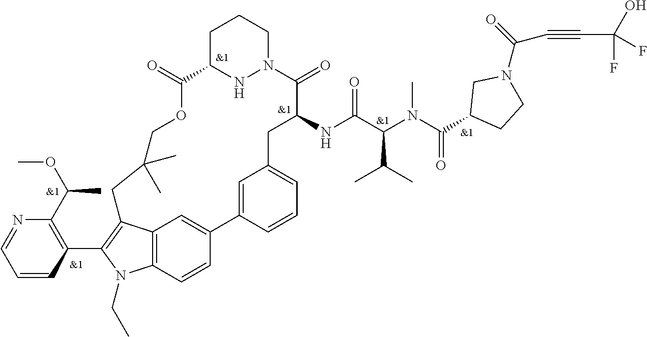 |
| B202 | 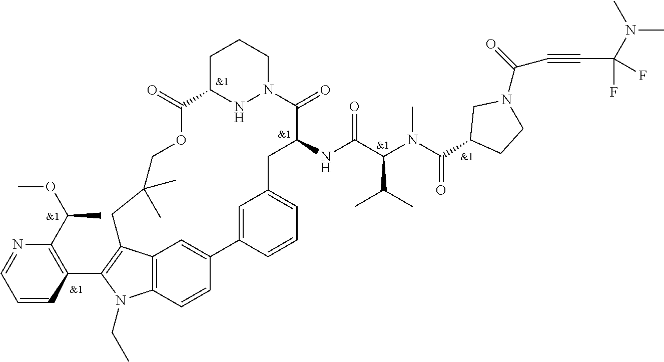 |
| B203 | 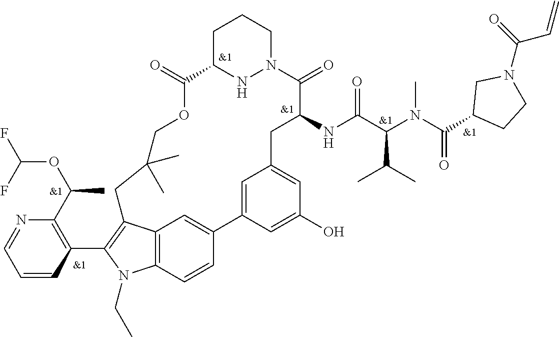 |
| B204 | 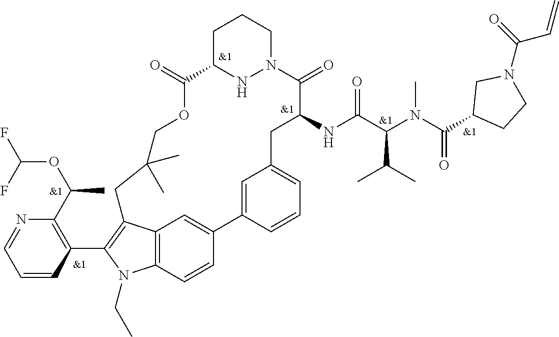 |
| B205 | 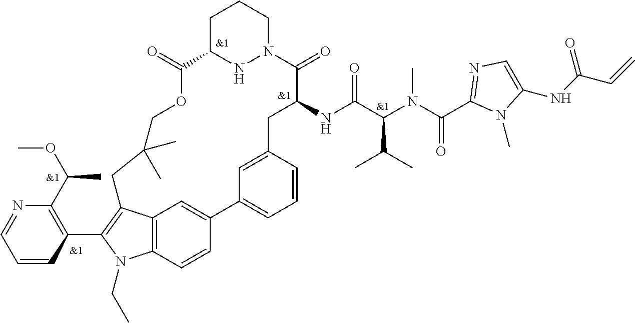 |
| B206 | 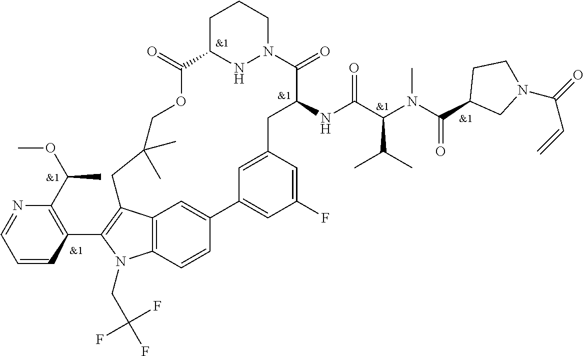 |
| B207 | 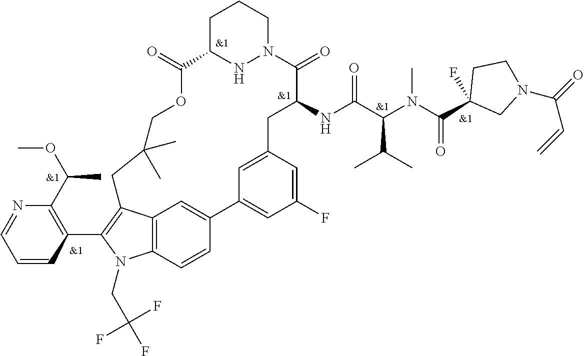 |
| B208 | 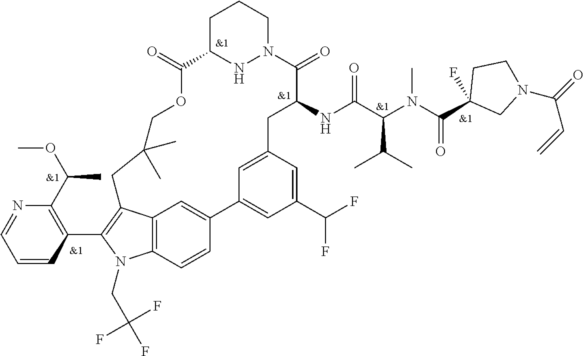 |
| B209 | 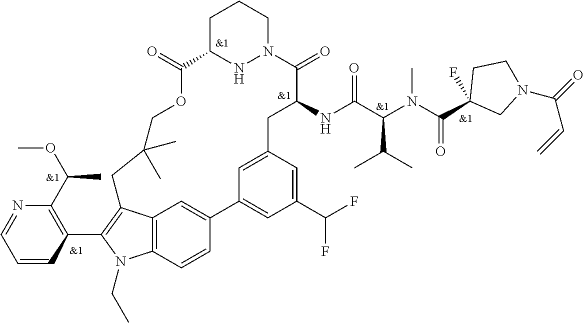 |
| B210 | 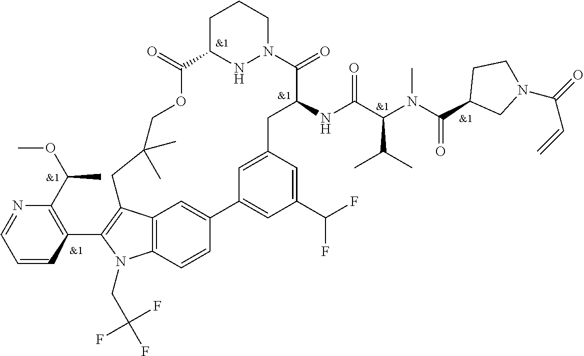 |
| B211 | 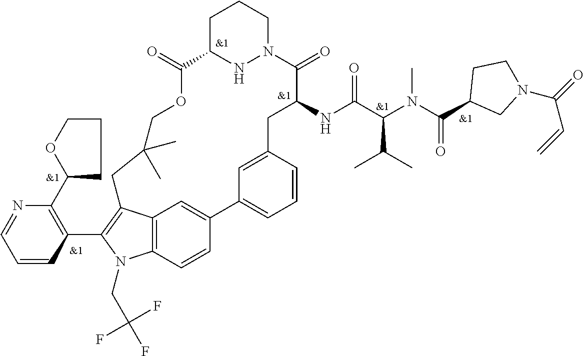 |
| B212 | 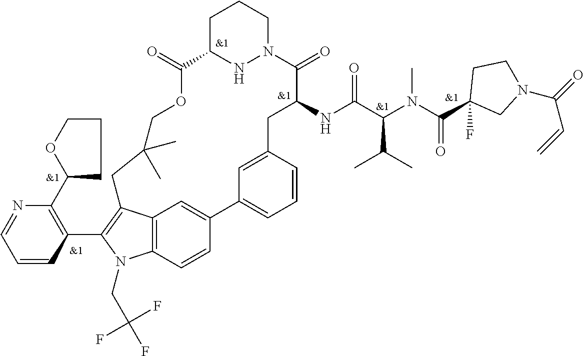 |
| B213 | 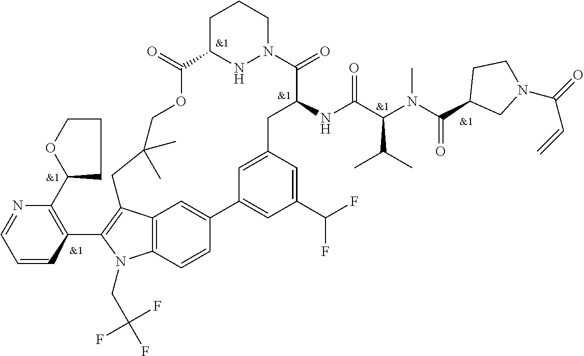 |
| B214 | 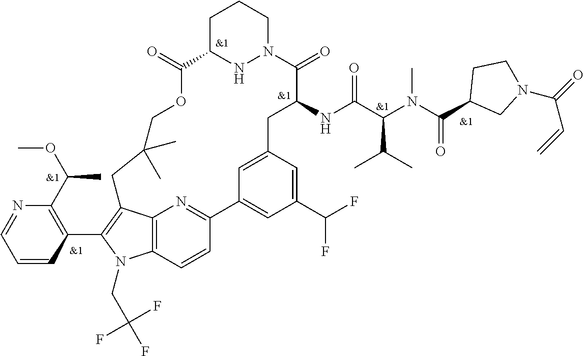 |
| B215 | 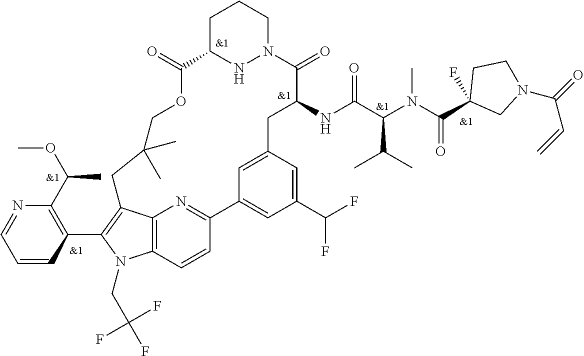 |
| B216 | 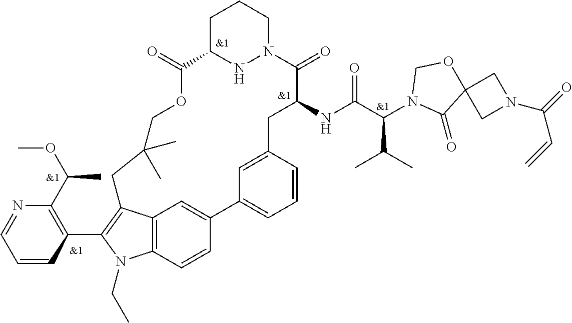 |
| B217 | 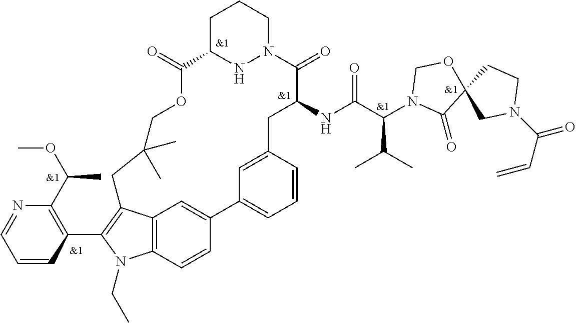 |
| B218 | 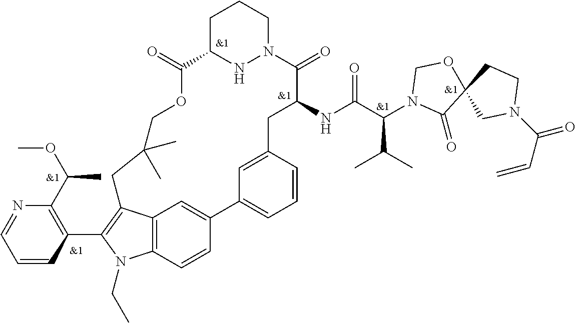 |
| B219 | 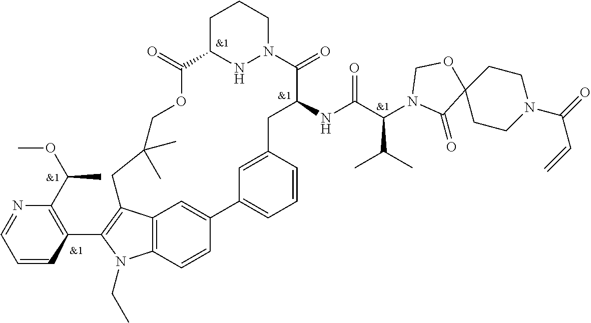 |
| B220 | 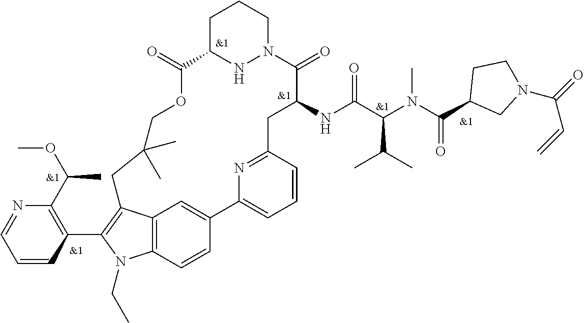 |
| B221 | 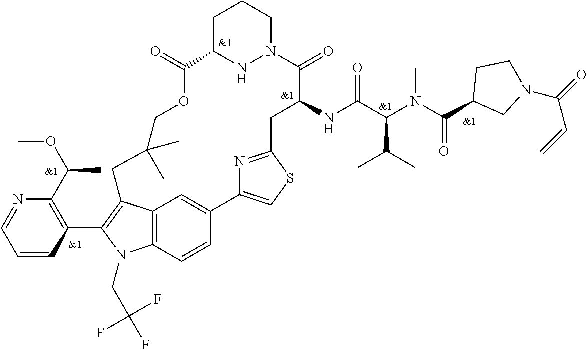 |
| B222 | 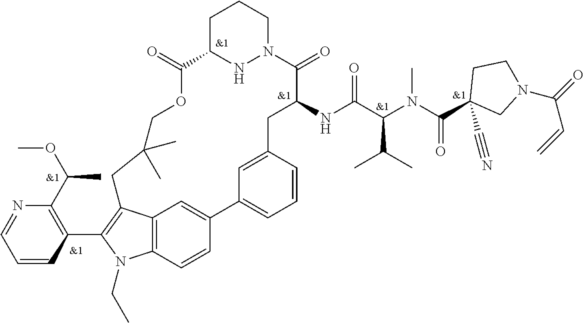 |
| B223 | 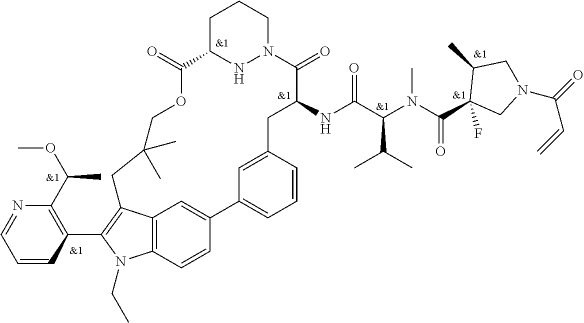 |
| B224 | 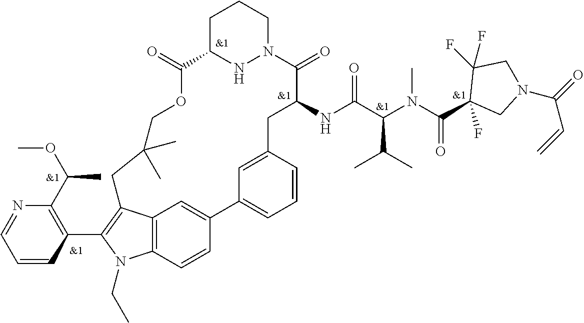 |
| B225 | 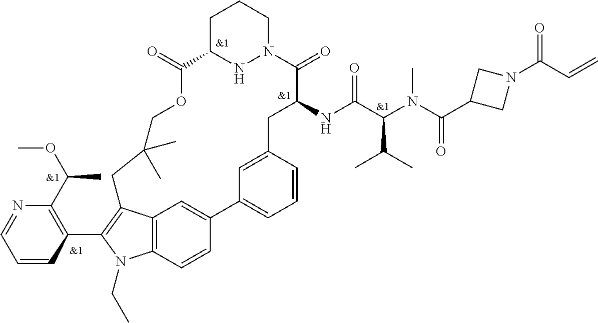 |
| B226 | 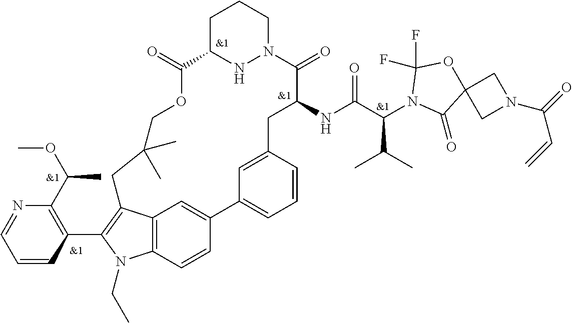 |
| B227 | 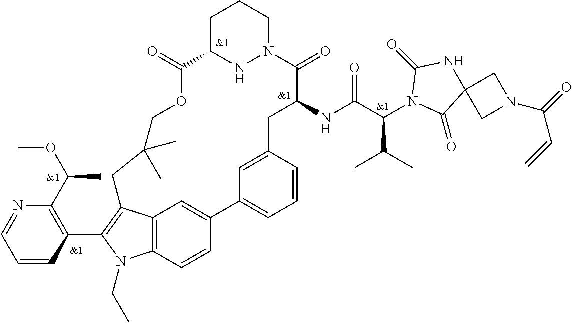 |
| B228 | 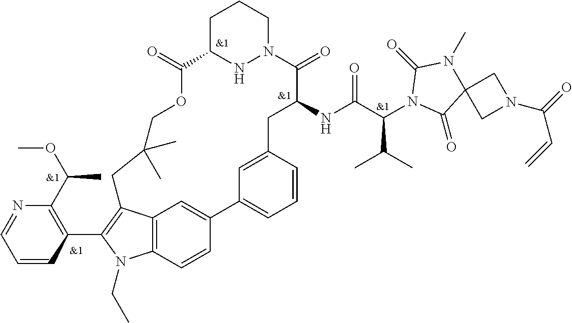 |
| B229 | 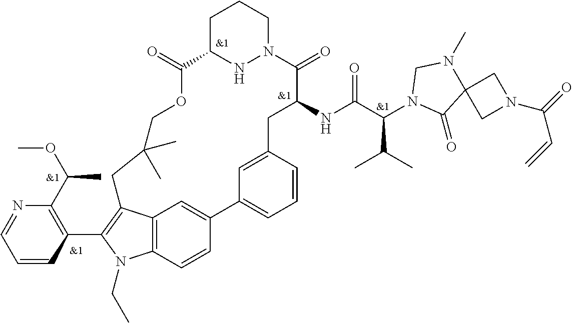 |
| B230 | 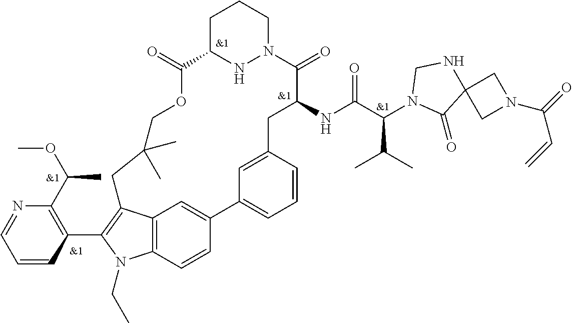 |
| B231 | 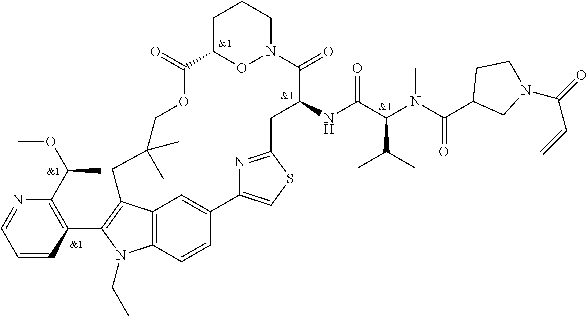 |
| B232 | 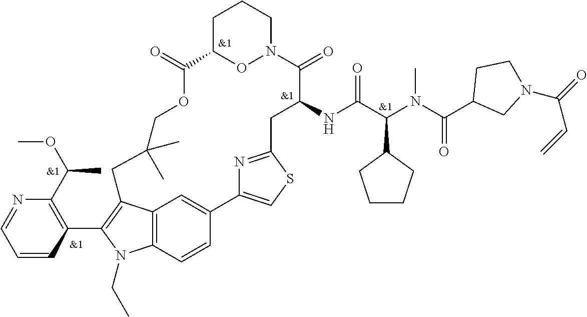 |
| B233 | 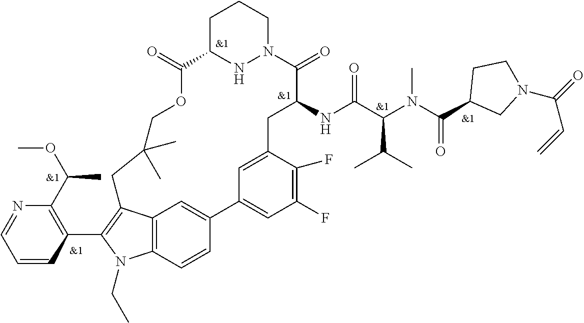 |
| B234 | 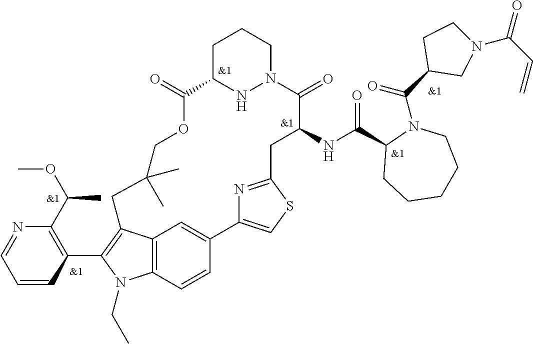 |
| B235 | 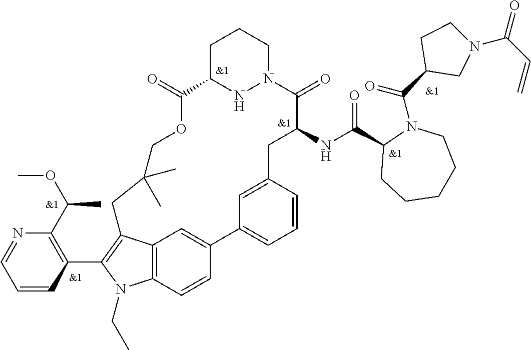 |
| B236 | 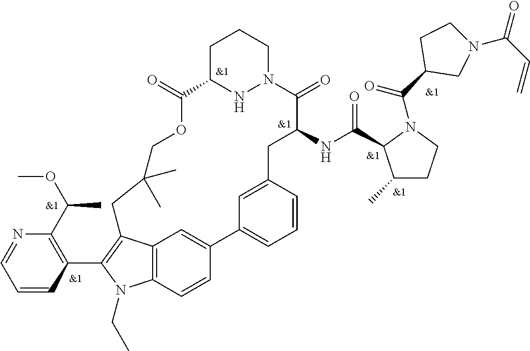 |
| B237 | 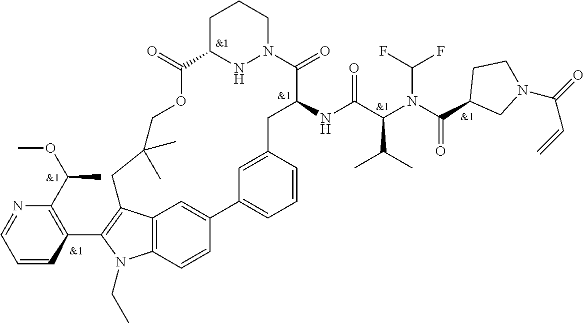 |
| B238 | 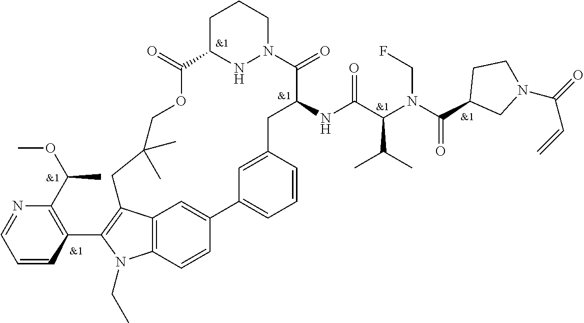 |
| B239 | 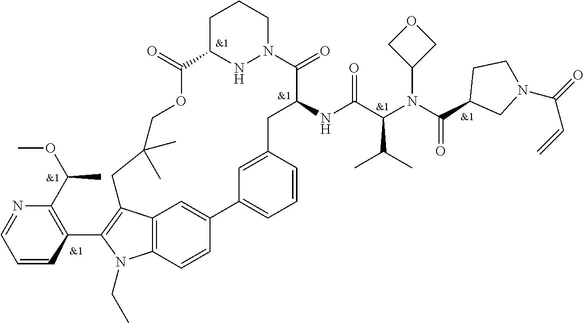 |
| B240 | 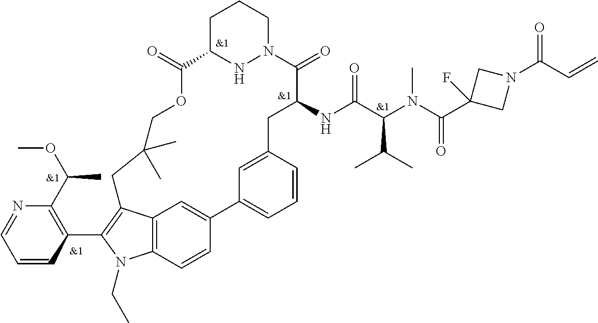 |
| B241 | 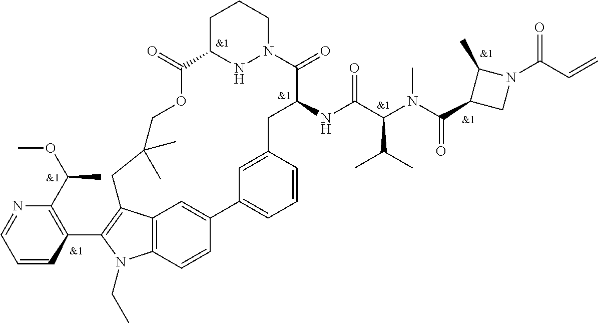 |
| B242 | 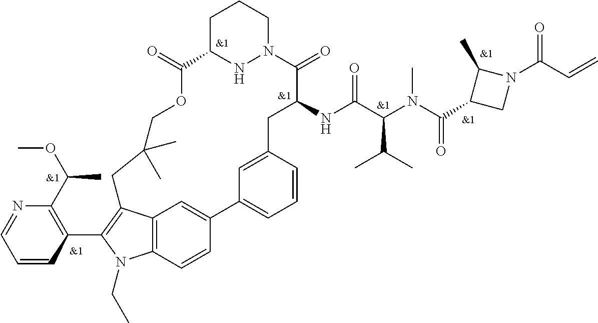 |
| B243 | 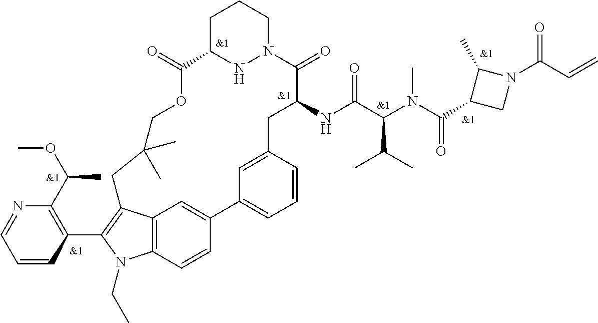 |
| B244 | 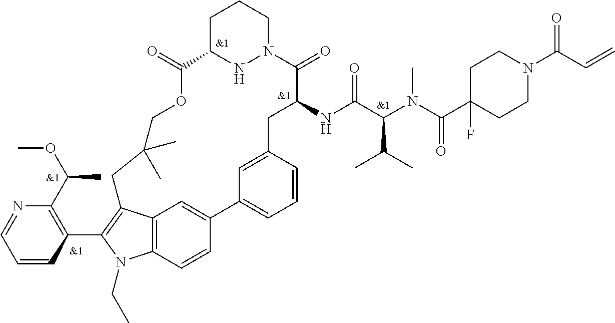 |
| B245 | 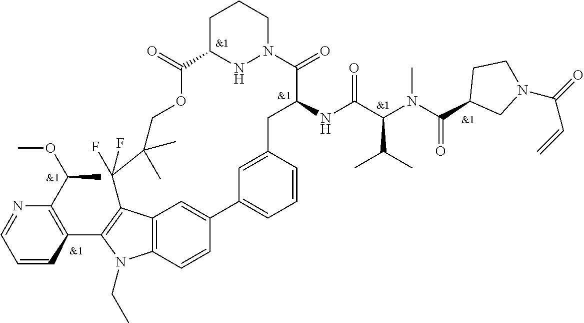 |
| B246 | 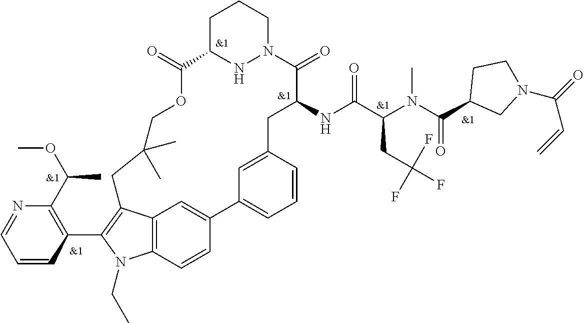 |
| B247 | 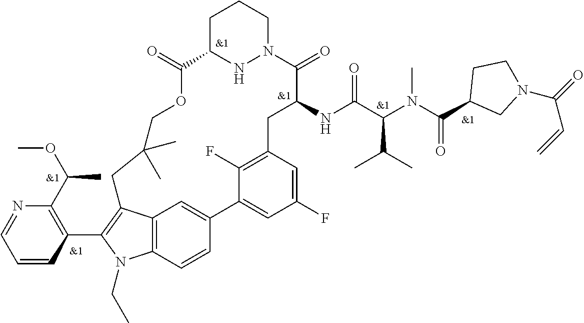 |
| B248 | 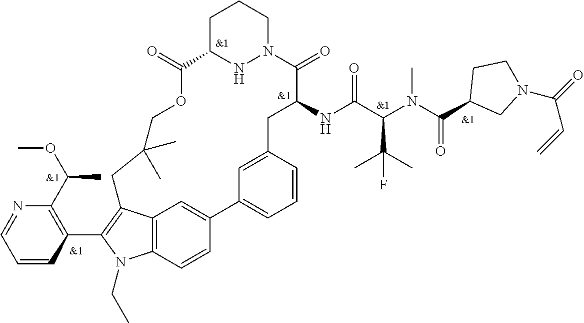 |
| B249 | 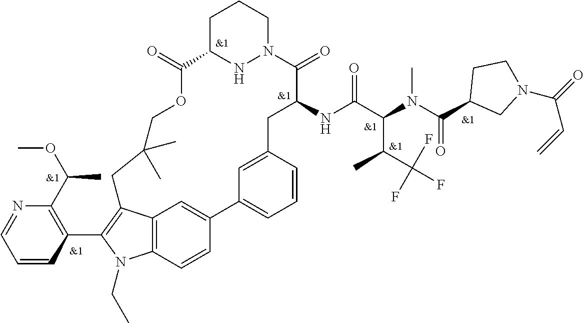 |
| B250 | 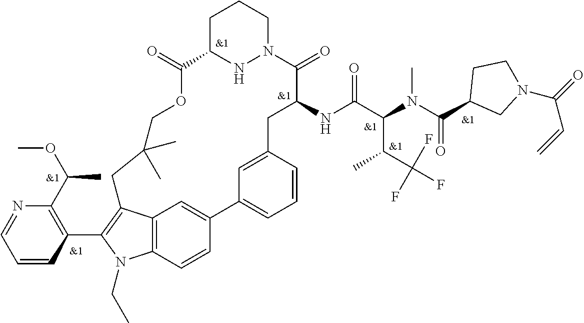 |
| B251 | 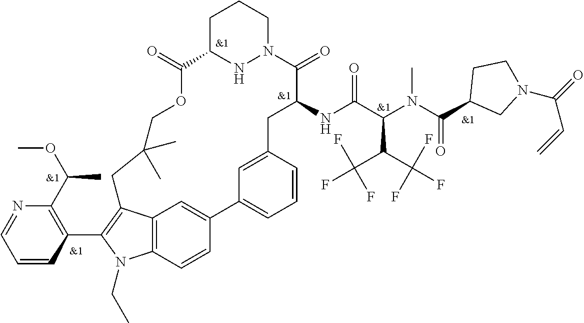 |
| B252 | 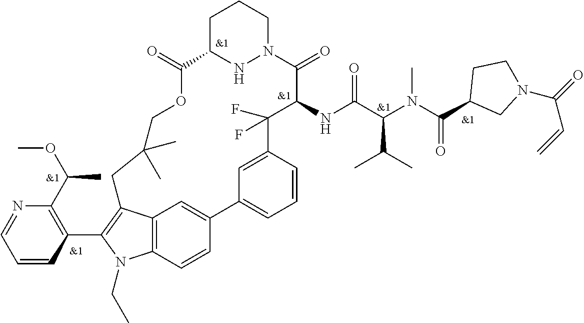 |
| B253 | 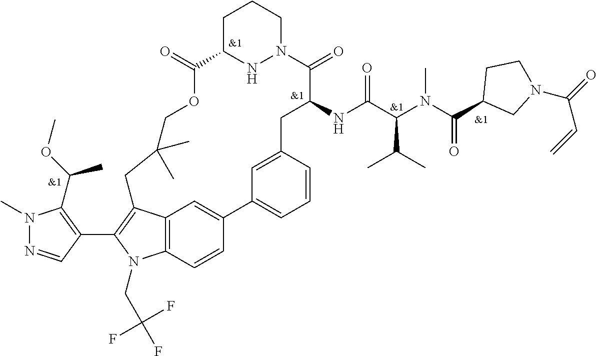 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| B254 | 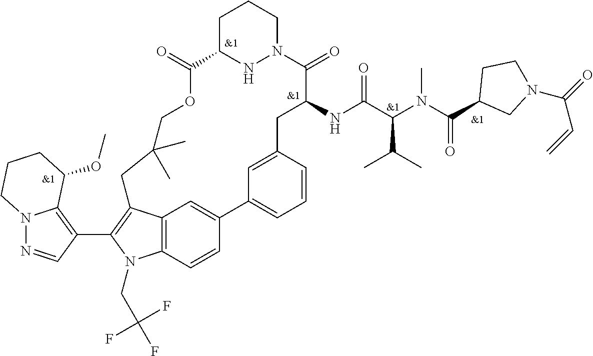 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| B255 | 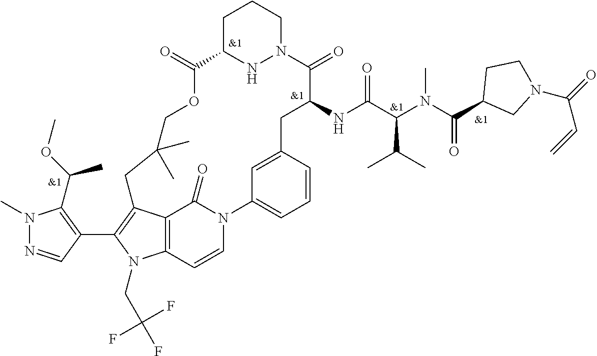 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| B256 | 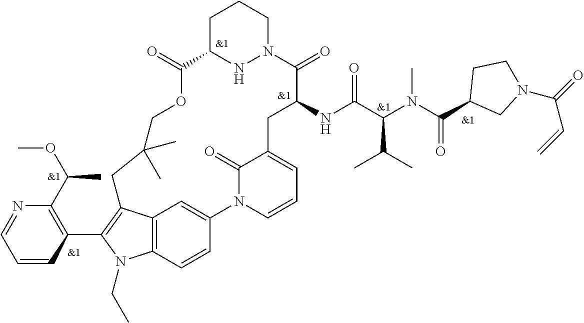 |
| B257 | 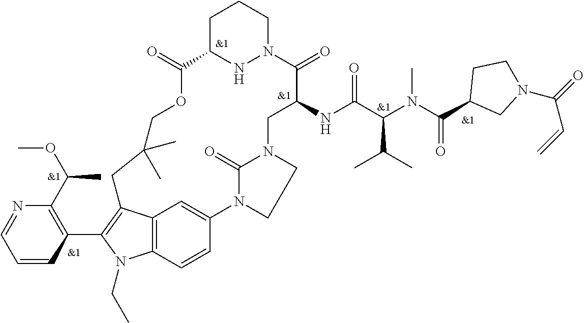 |
| B258 | 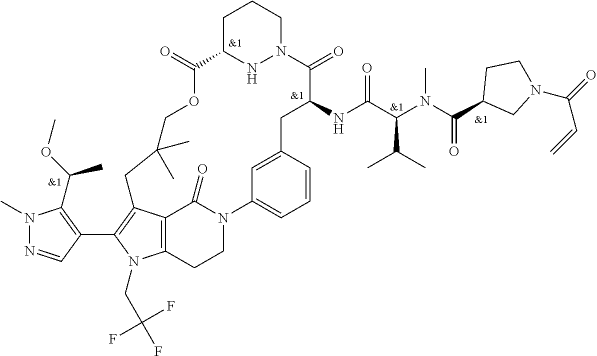 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| B259 | 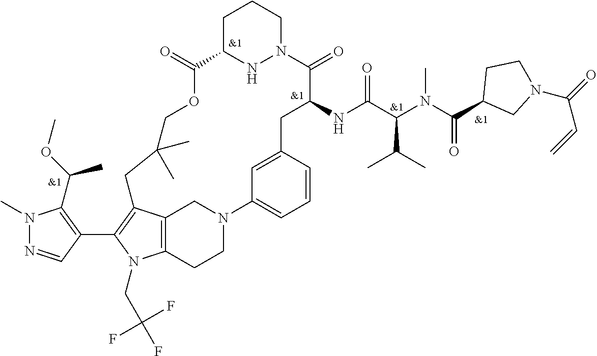 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| B260 | 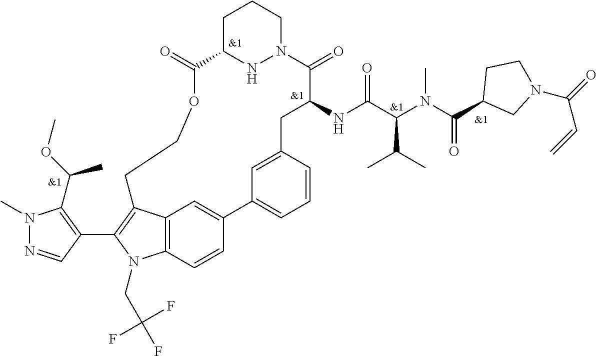 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| B261 | 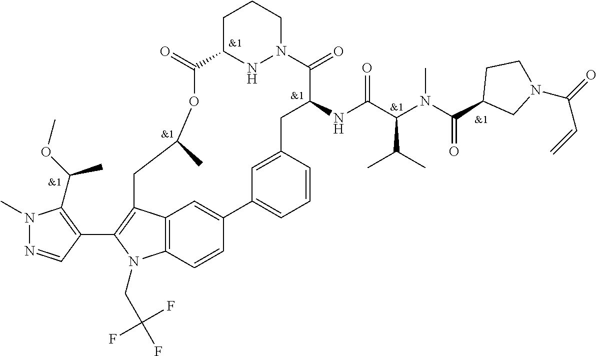 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| B262 | 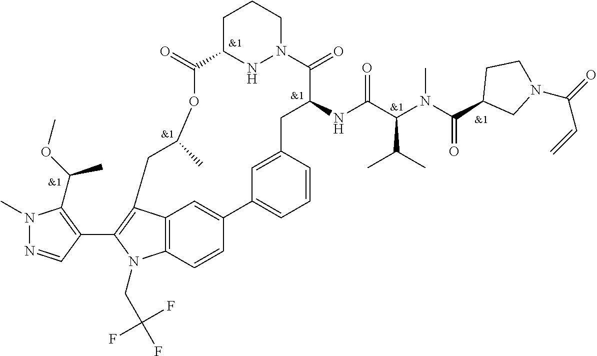 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| B263 | 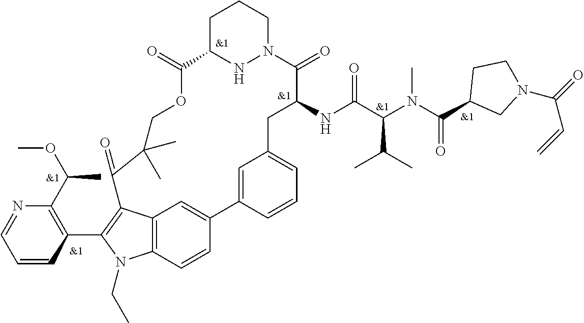 |
| B264 | 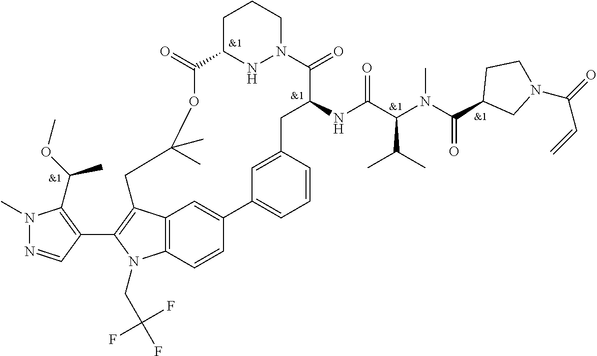 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)OC(=O)[C@@H]3CCCN(N3)C2=O)C(C)C)C1] |
| B265 | 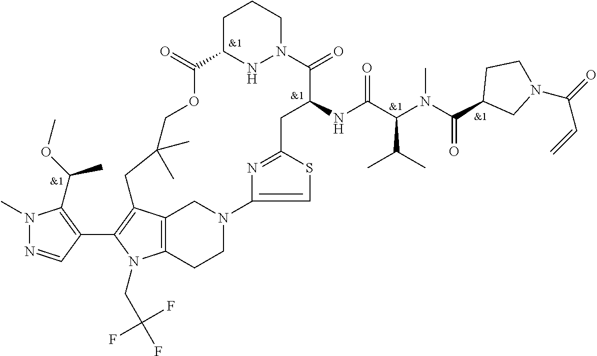 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| B266 | 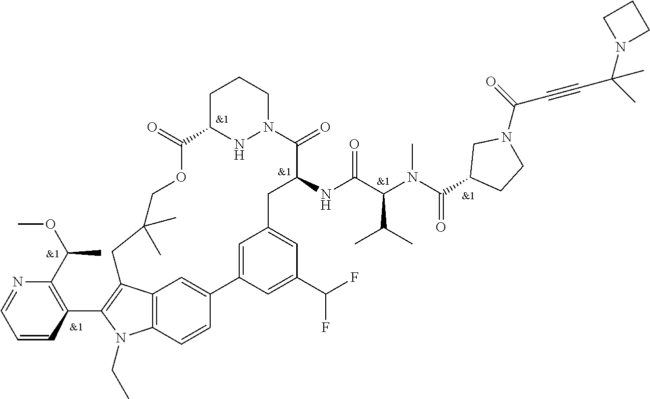 |
| B267 | 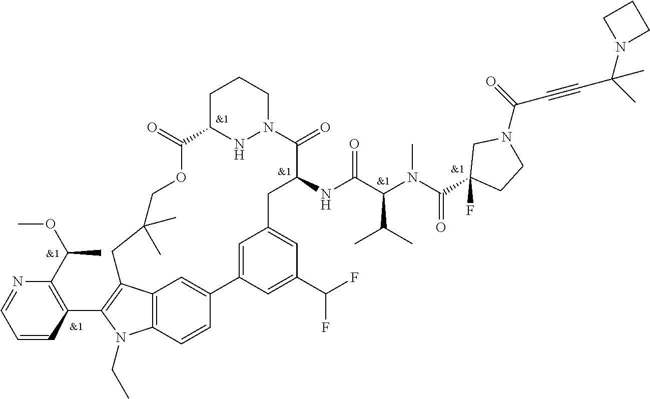 |
| B268 | 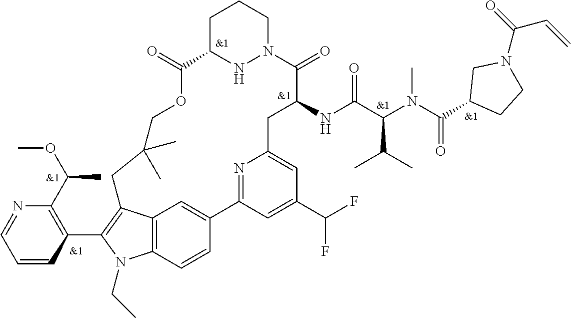 |
| B269 | 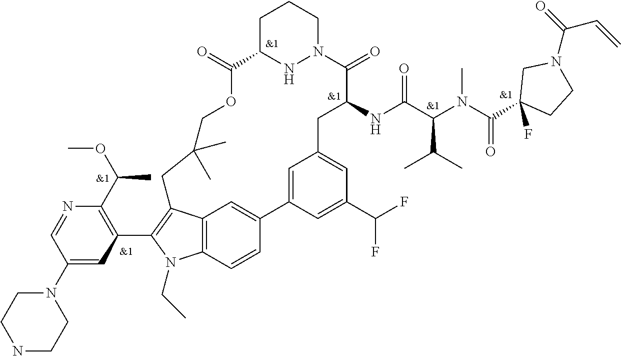 |
| B270 | 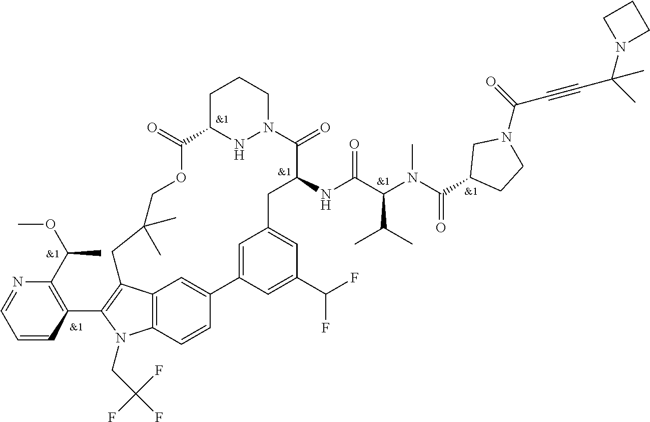 |
| B271 | 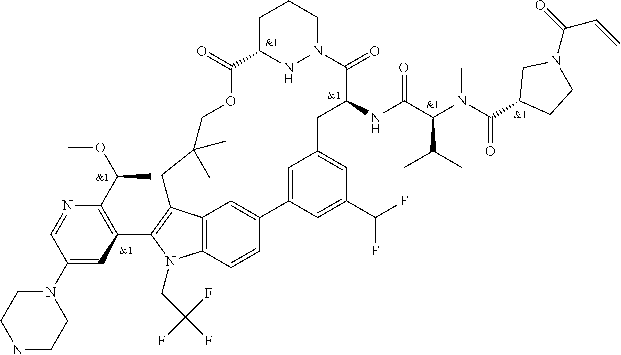 |
| B272 | 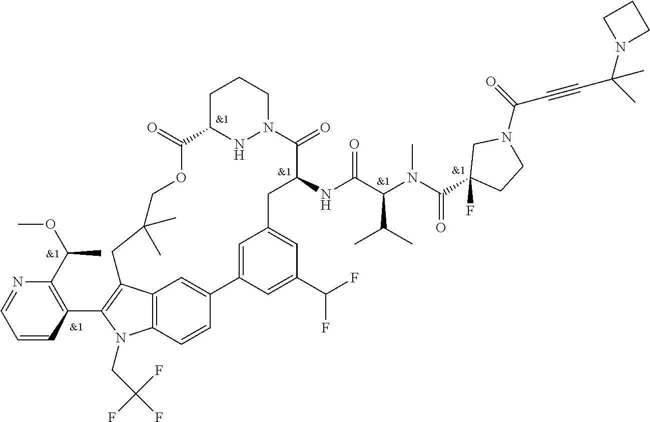 |
| B273 | 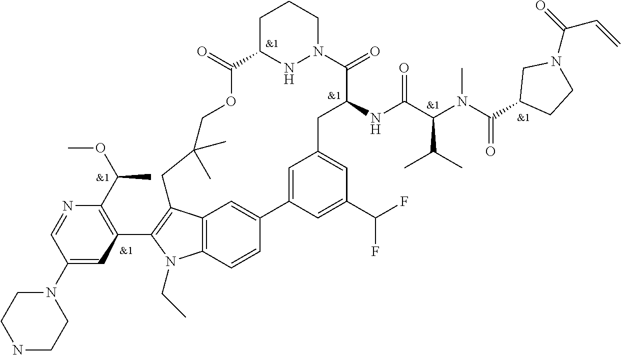 |
| B274 | 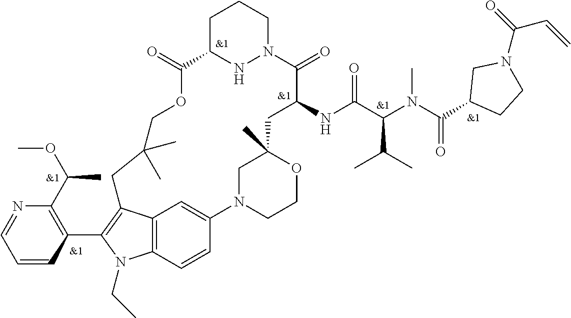 |
| B275 | 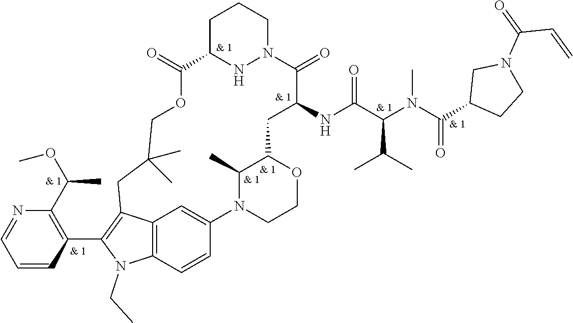 |
| B276 | 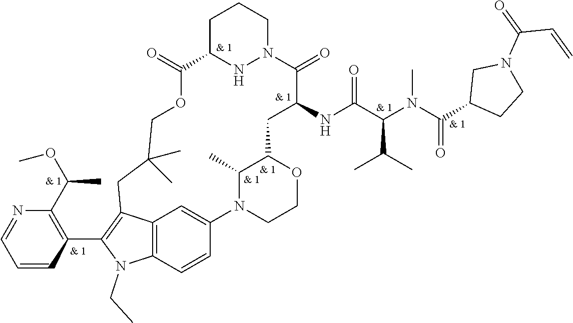 |
| B277 | 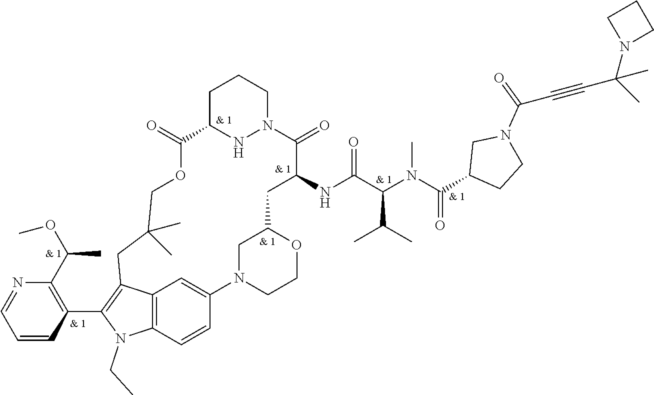 |
| B278 | 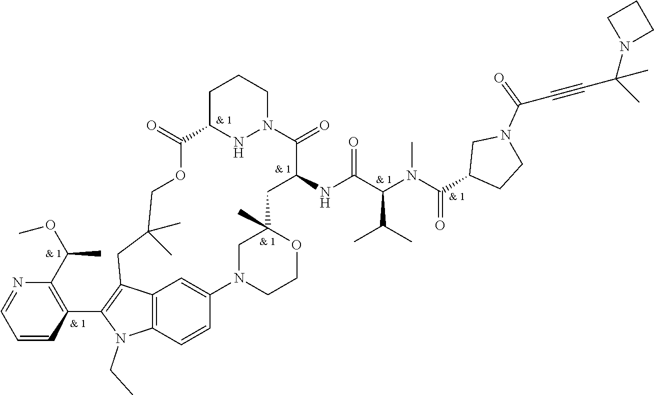 |
| B279 | 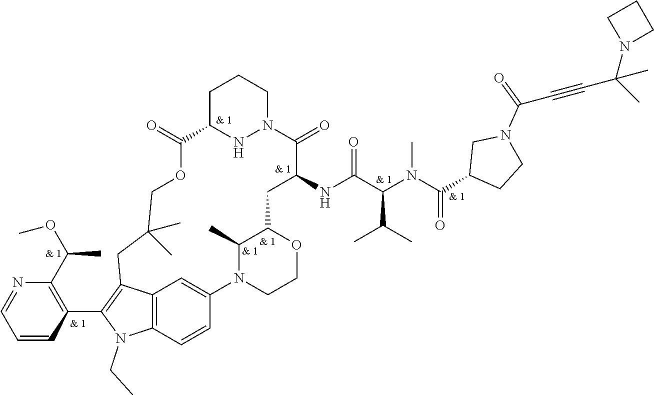 |
| B280 | 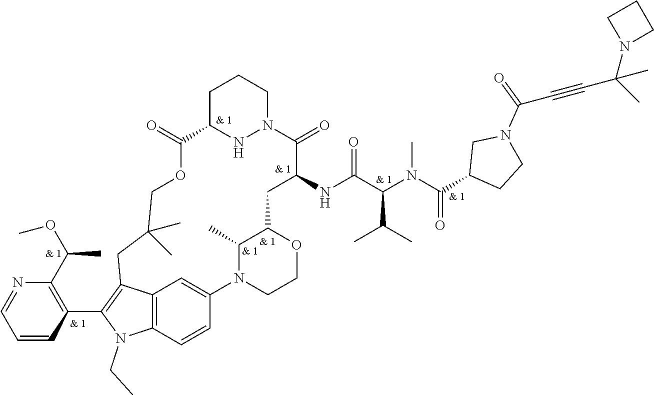 |
| B282 | 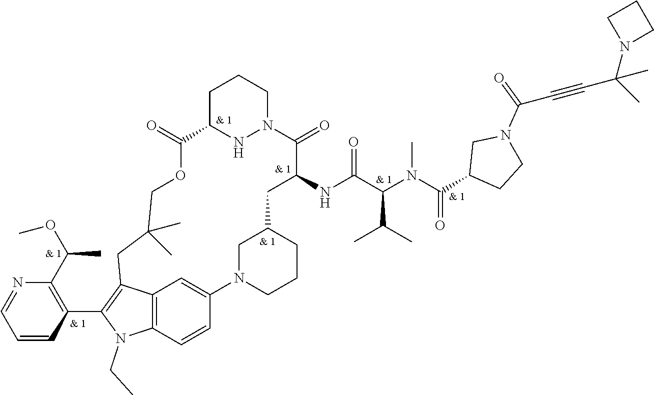 |
| B283 | 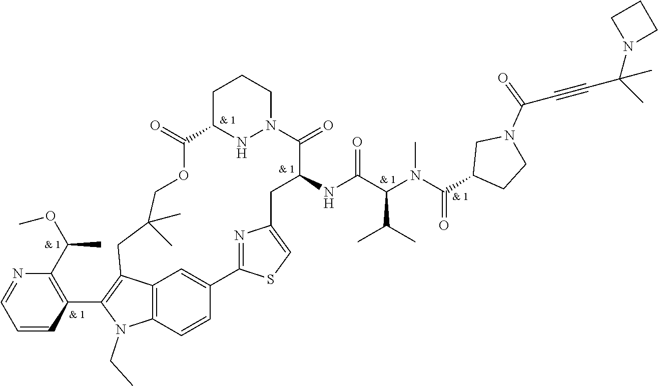 |
| B284 | 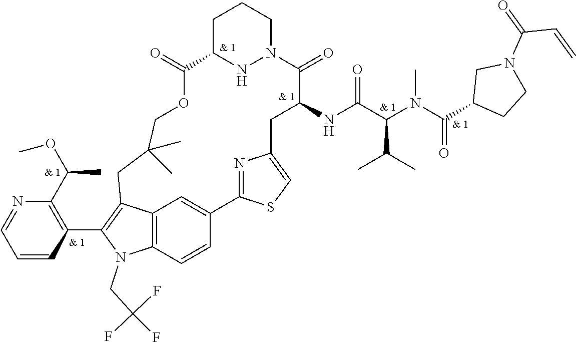 |
| B285 | 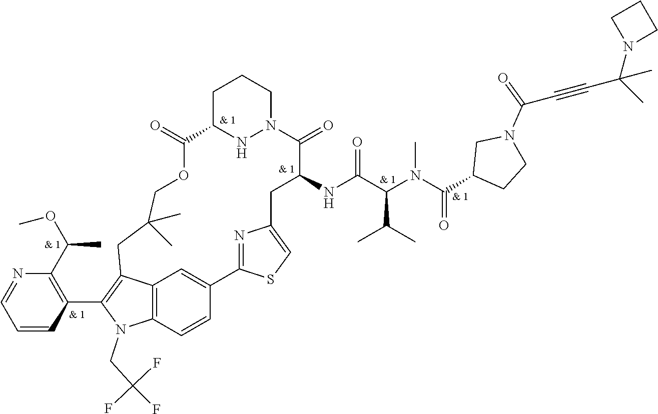 |
| B286 | 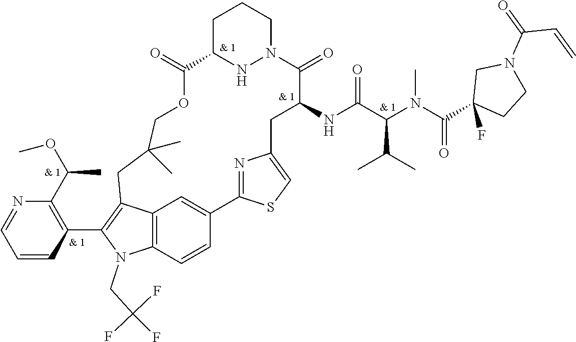 |
| B287 | 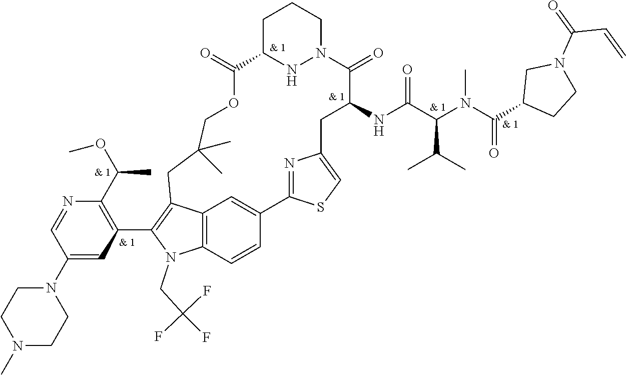 |
| B288 | 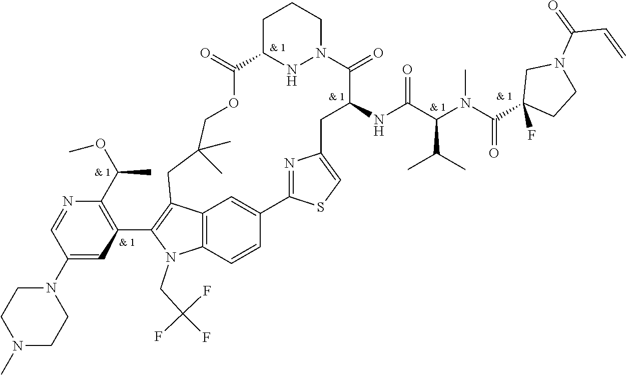 |
| B289 | 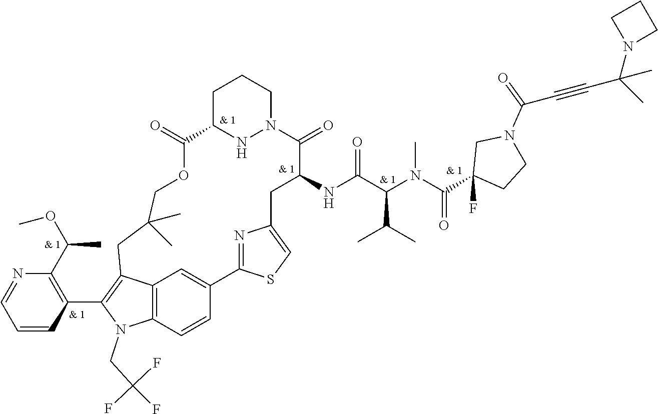 |
| B290 | 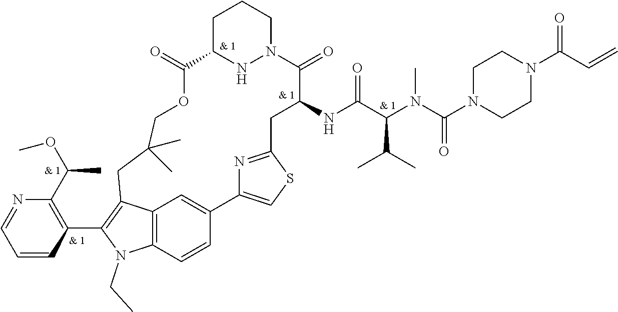 |
| B291 | 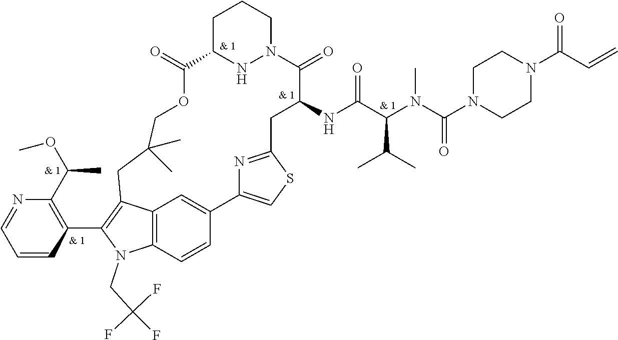 |
| B292 | 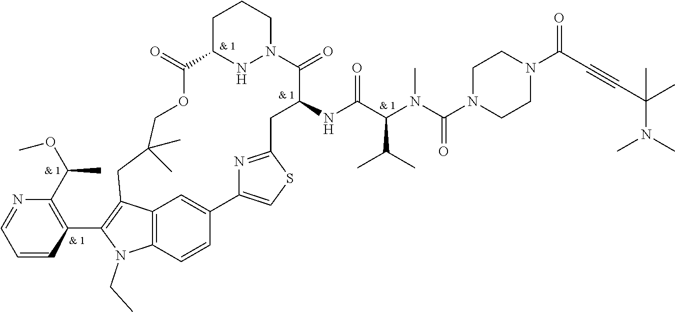 |
| B293 | 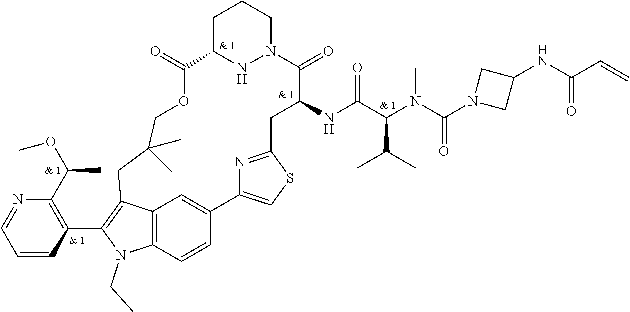 |
| B294 | 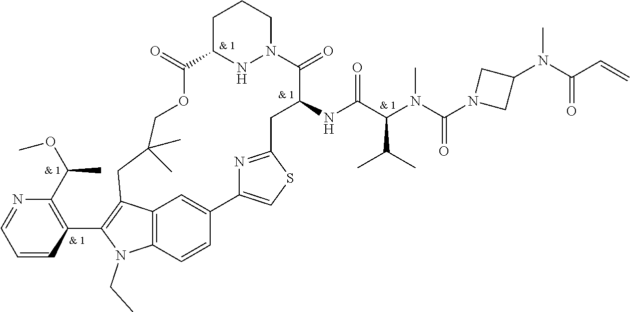 |
| B295 | 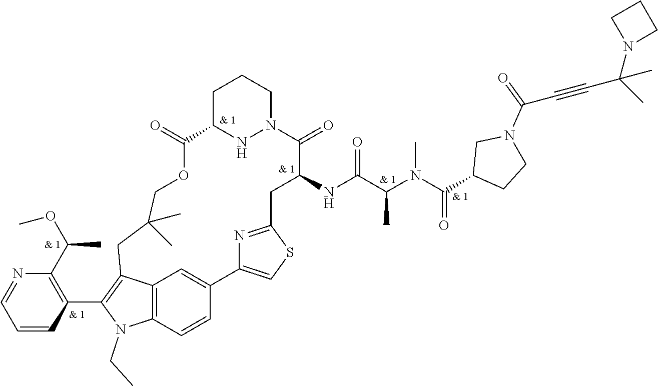 |
| B296 | 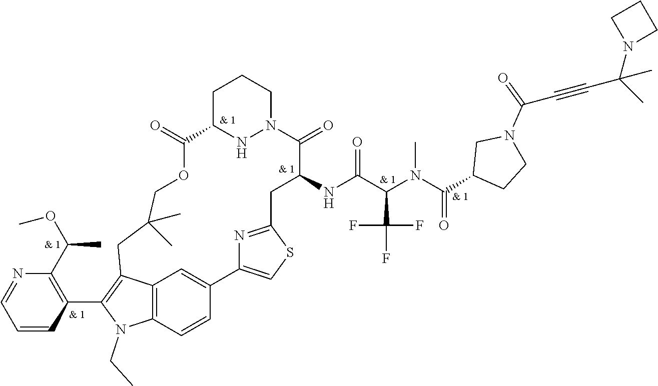 |
| B297 | 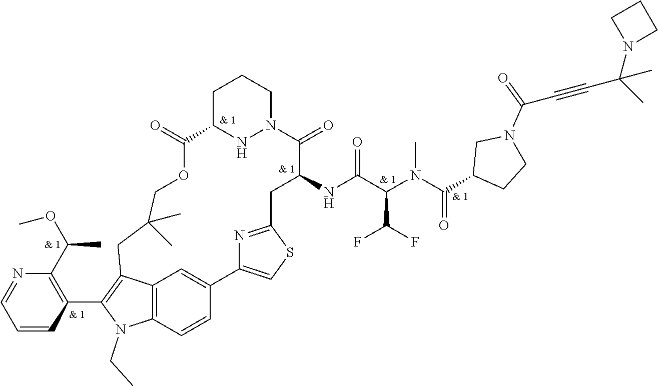 |
| B298 | 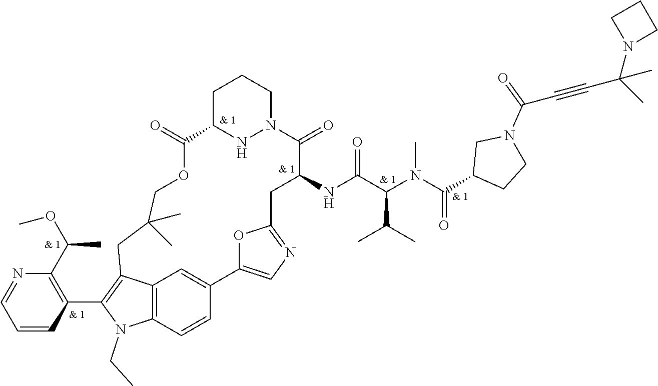 |
| B299 | 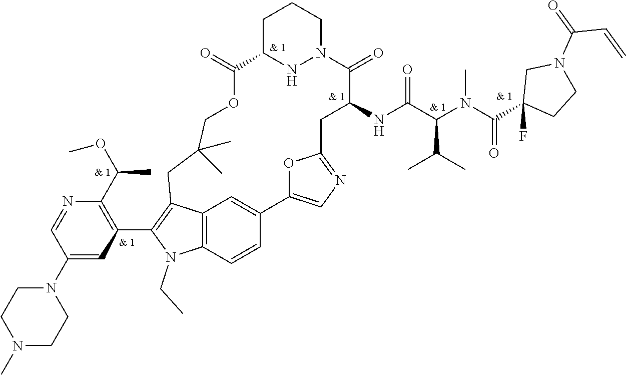 |
| B300 | 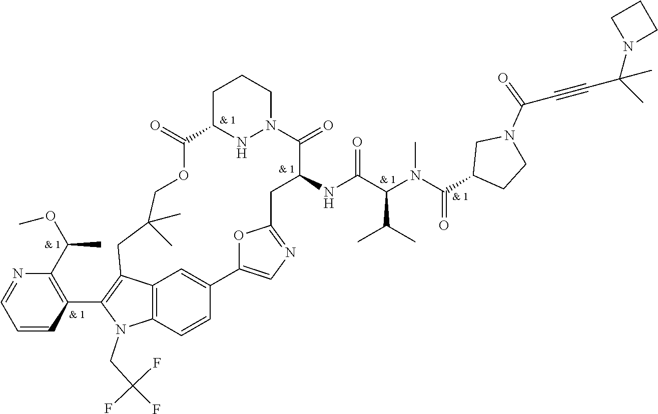 |
| B301 | 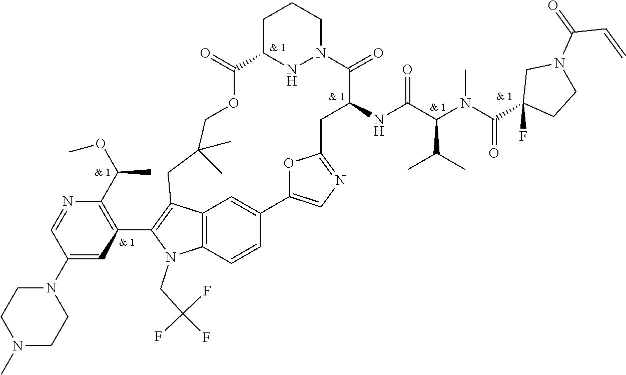 |
| B302 | 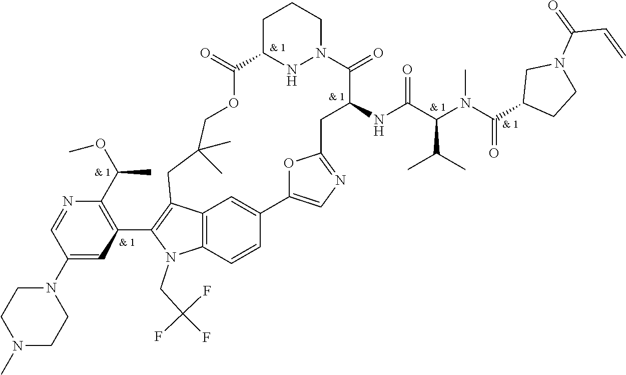 |
| B303 | 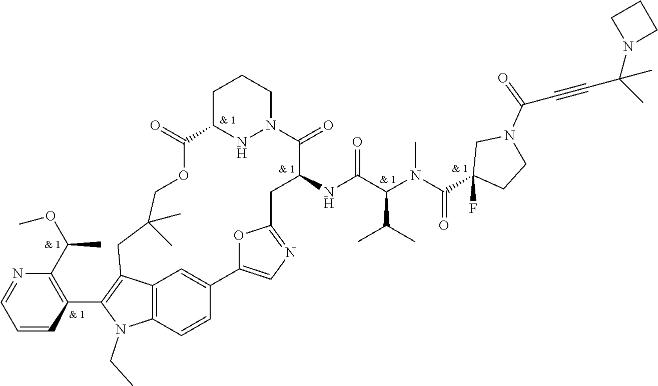 |
| B304 | 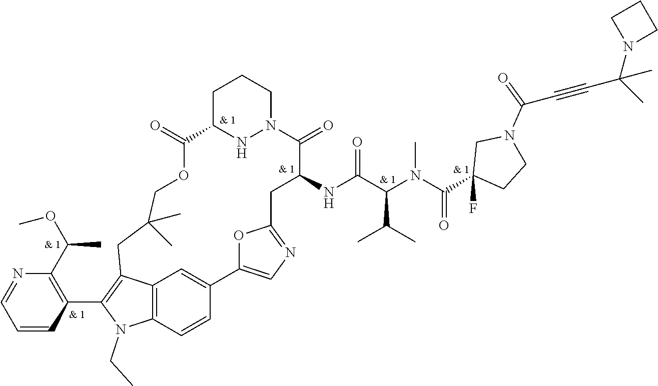 |
| B305 | 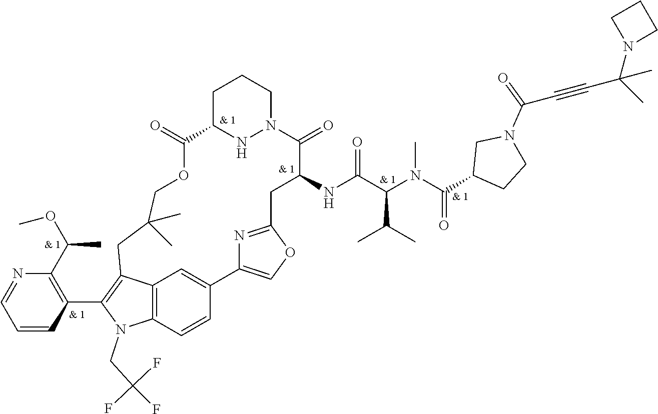 |
| B306 | 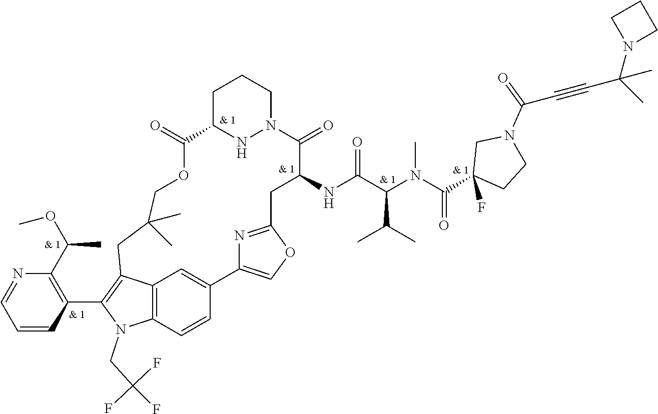 |
| B307 | 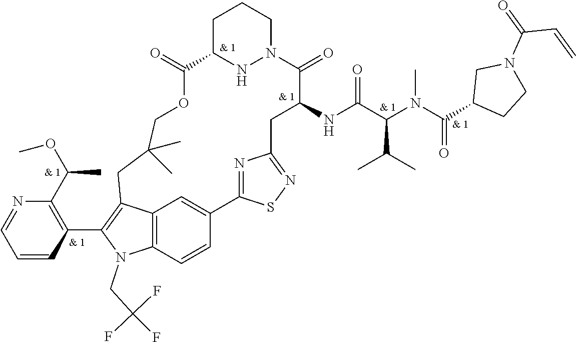 |
| B308 | 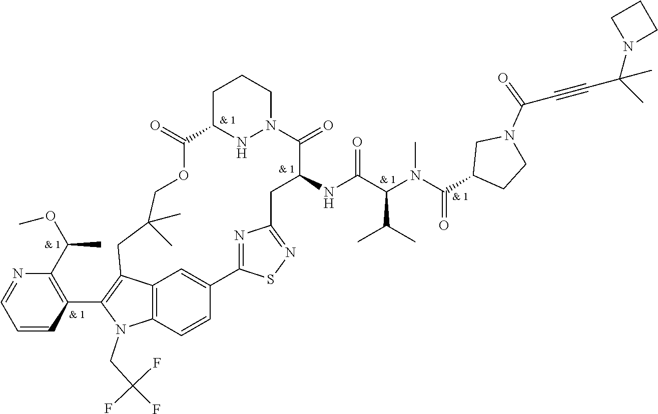 |
| B309 | 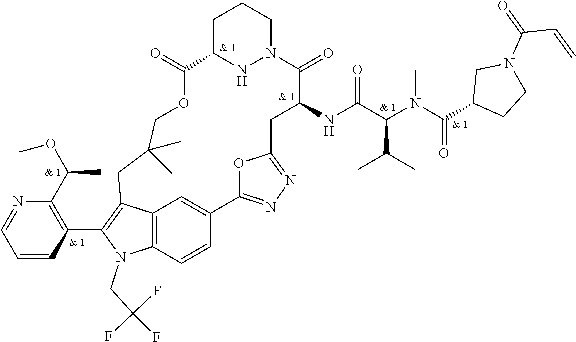 |
| B310 | 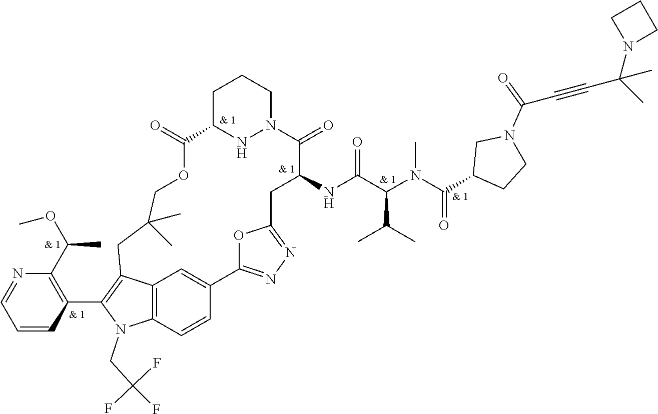 |
| B311 | 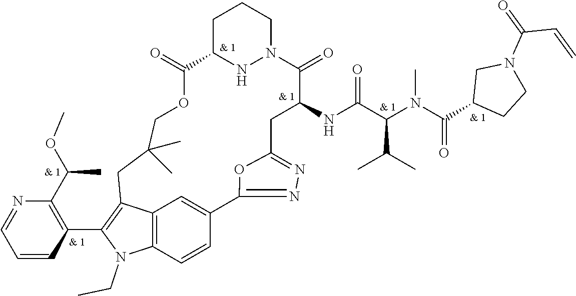 |
| B312 | 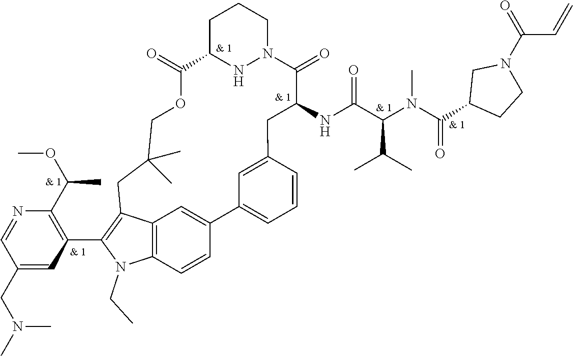 |
| B313 | 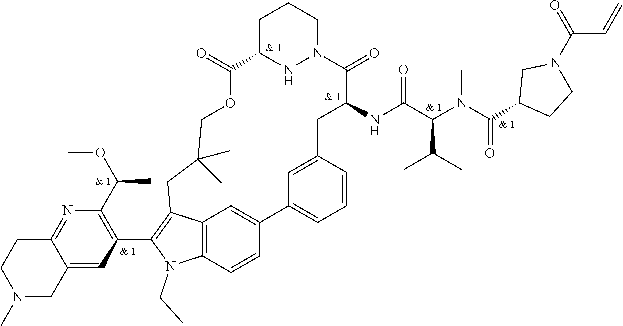 |
| B314 | 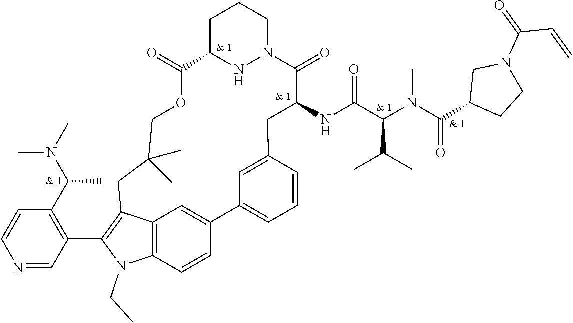 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| B315 | 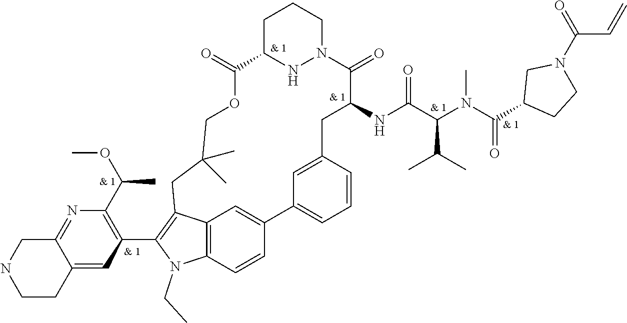 |
| B316 | 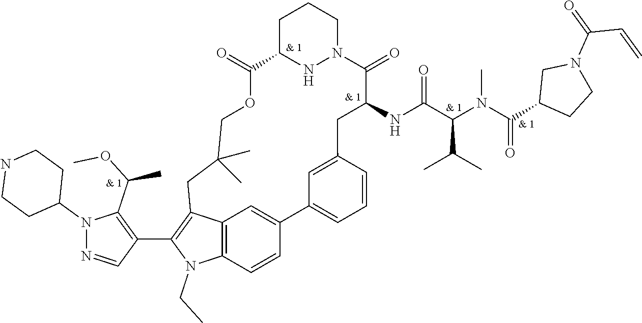 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| B317 | 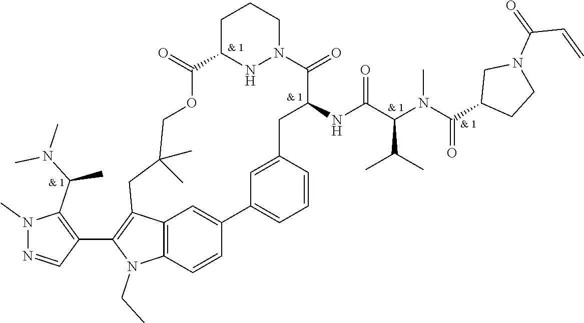 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| B318 | 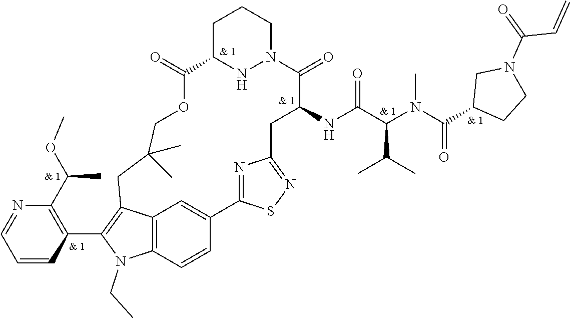 |
| B319 | 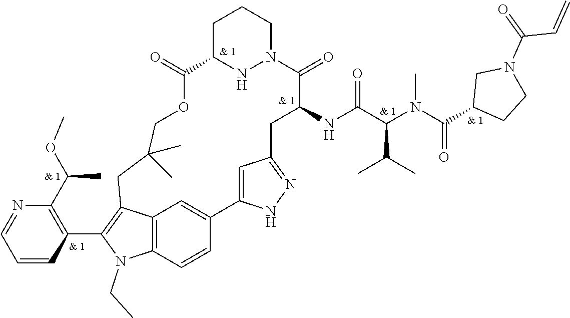 |
| B320 | 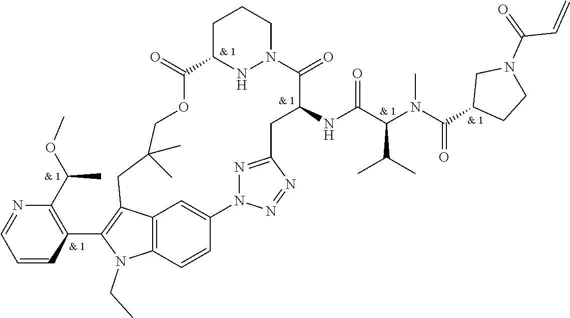 |
| B321 | 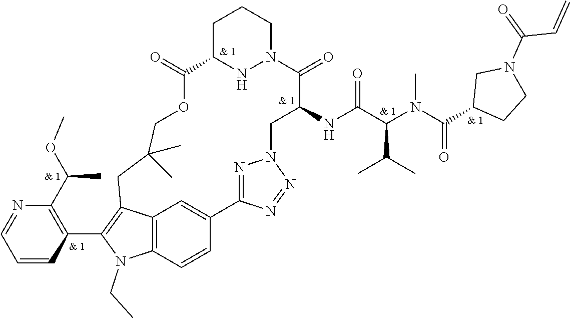 |
| B322 | 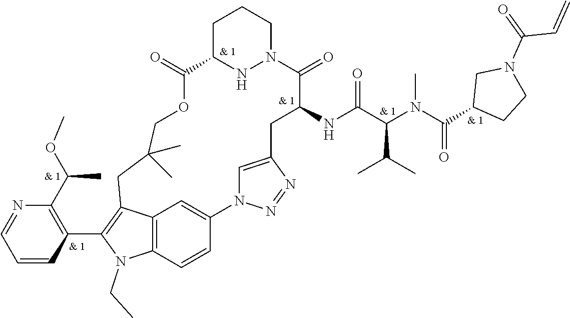 |
| B323 | 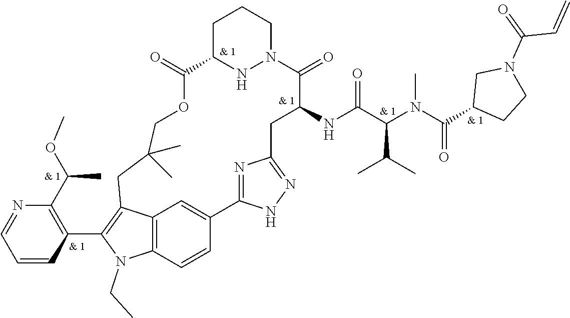 |
| B324 | 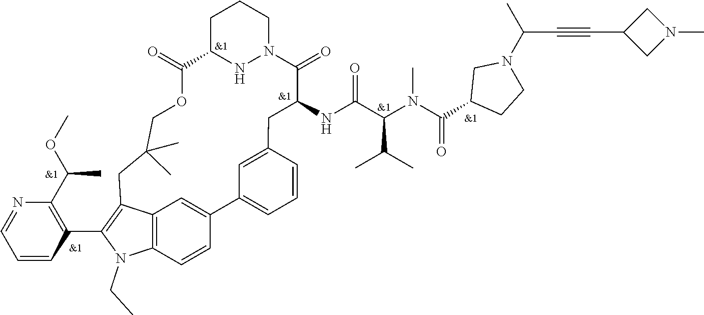 |
| B325 | 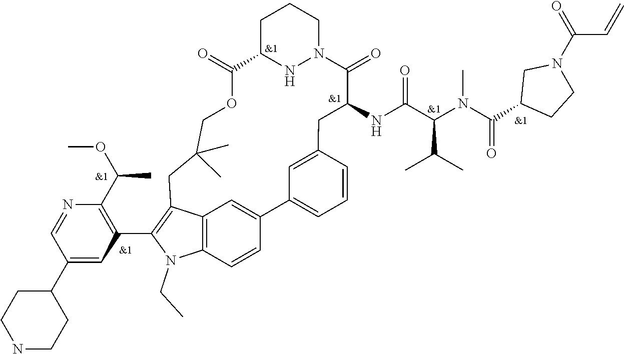 |
| B326 | 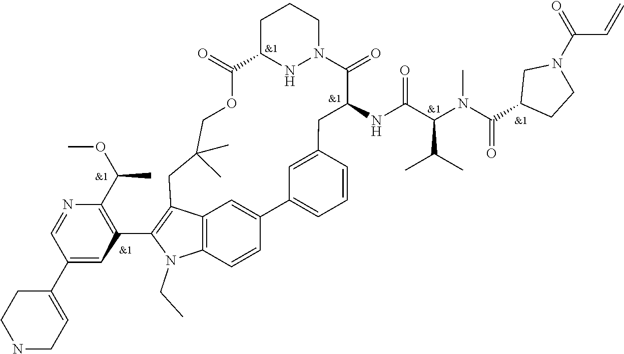 |
| B327 | 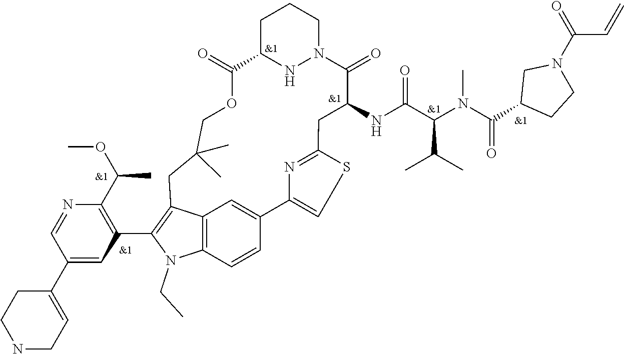 |
| B328 | 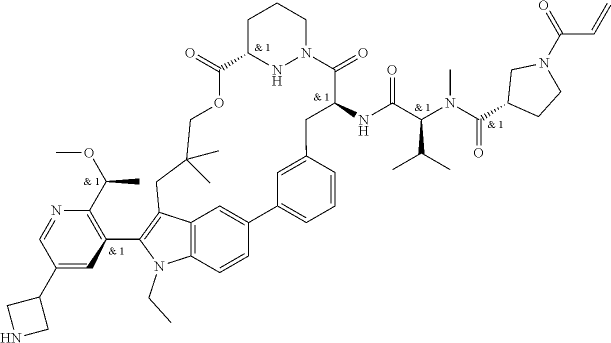 |
| B329 | 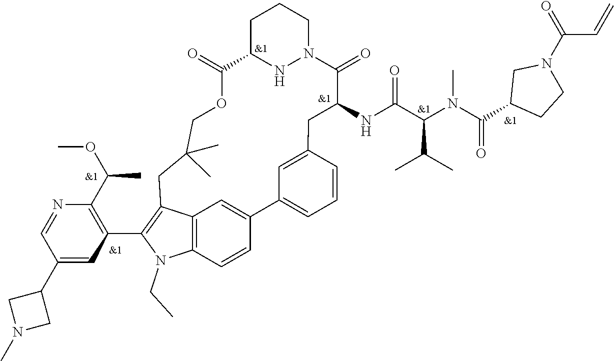 |
| B330 | 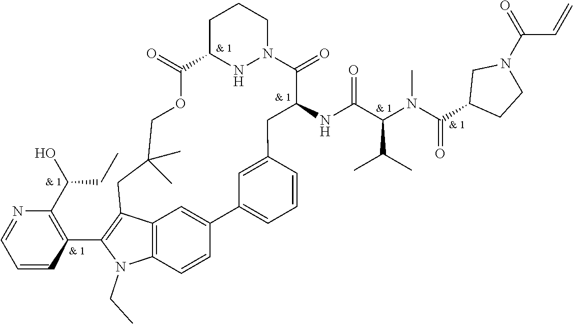 |
| B331 | 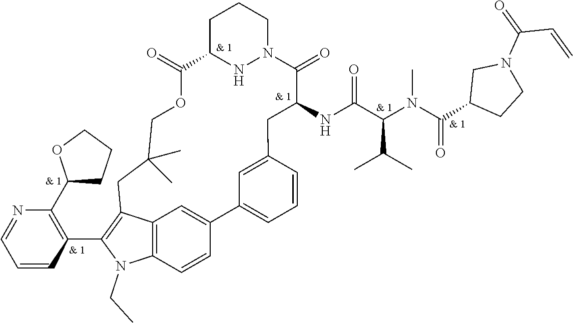 |
| B332 | 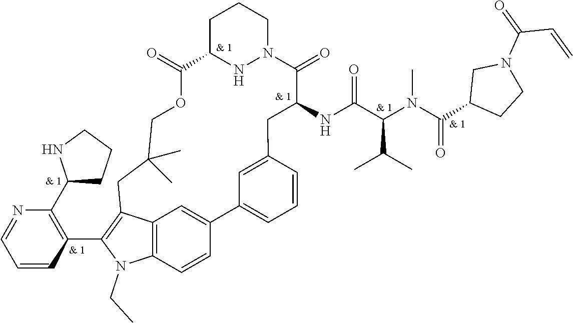 |
| B333 | 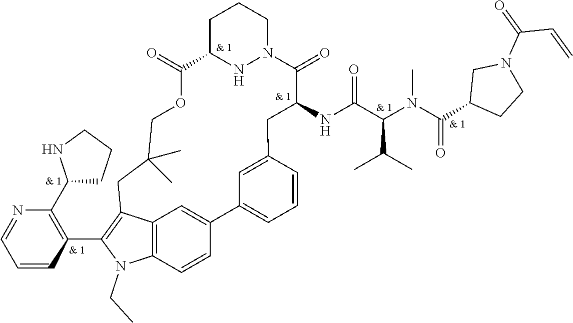 |
| B334 | 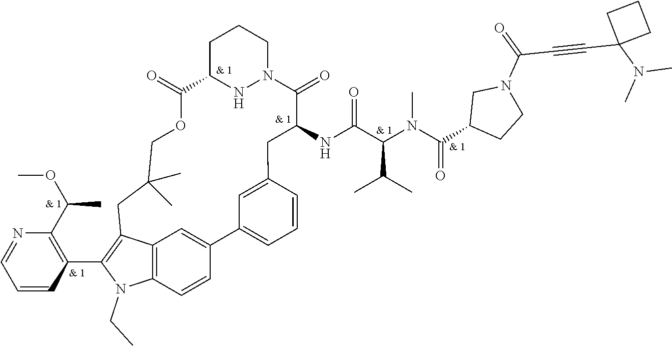 |
| B335 | 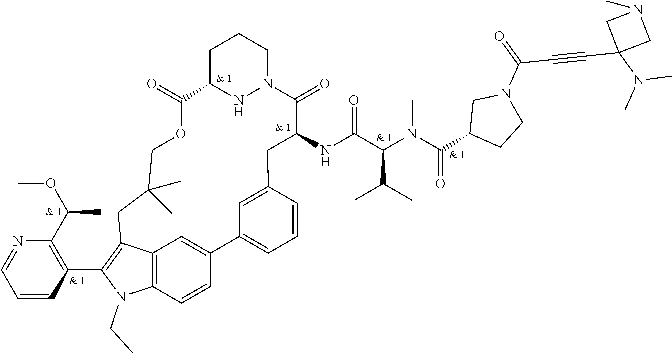 |
| B336 | 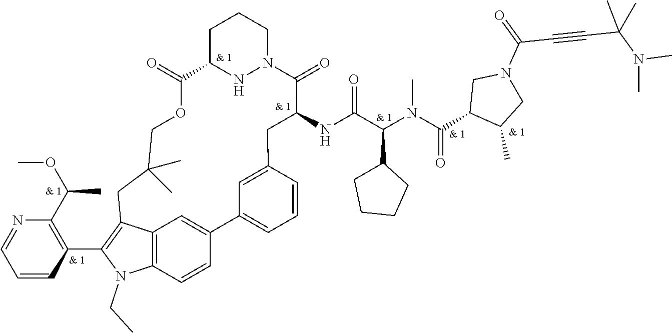 |
| B337 | 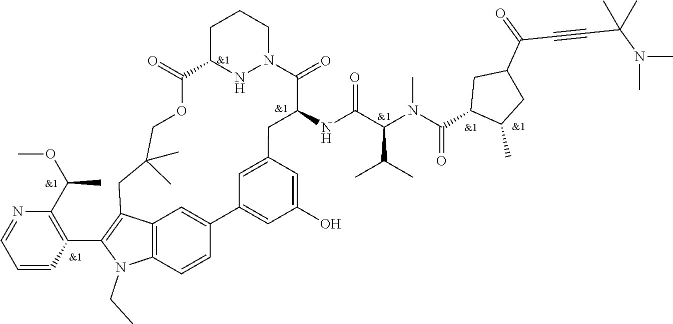 |
| B338 | 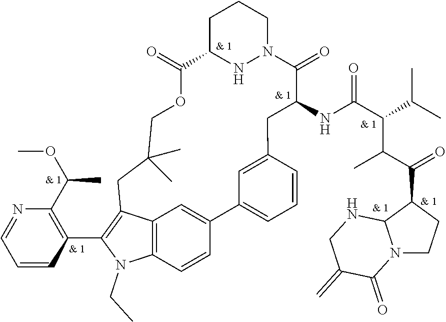 |
| B339 | 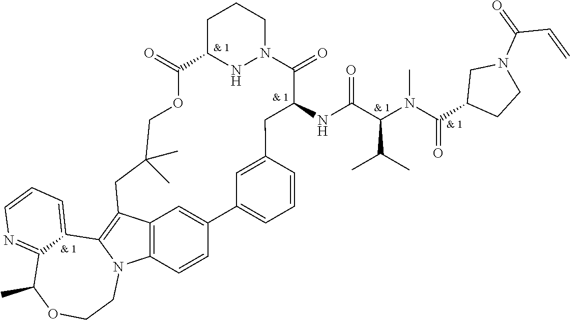 |
| B340 | 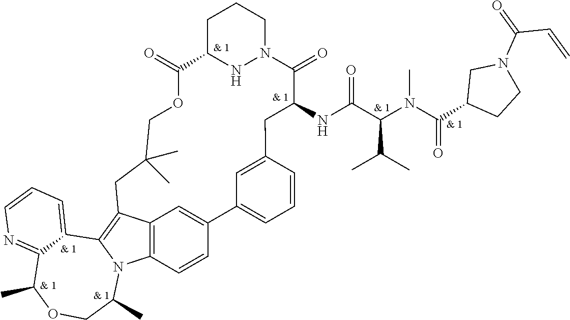 |
| B341 | 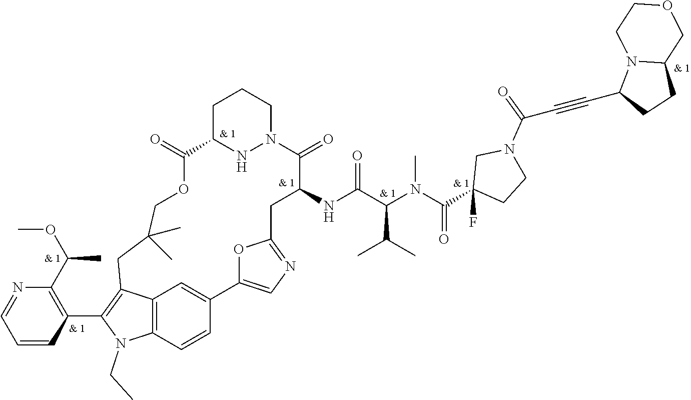 |
| B342 | 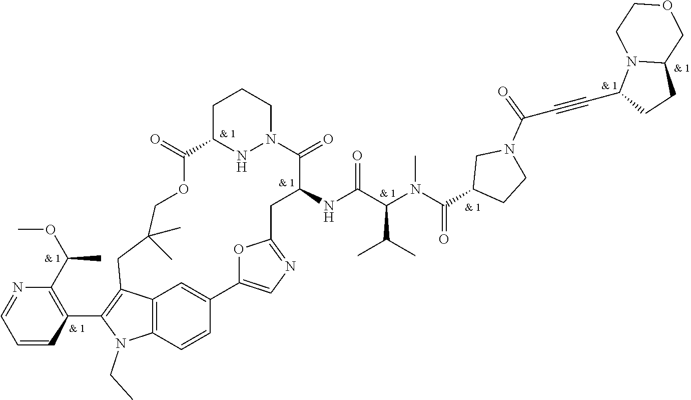 |
| B343 | 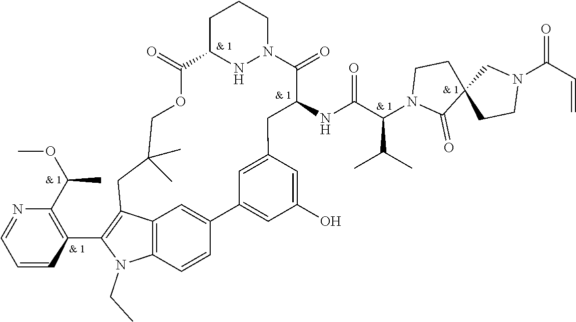 |
| B344 | 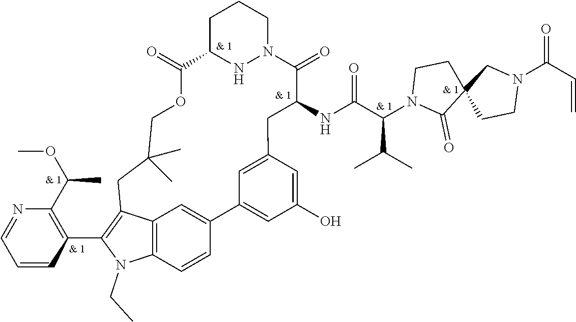 |
| B345 | 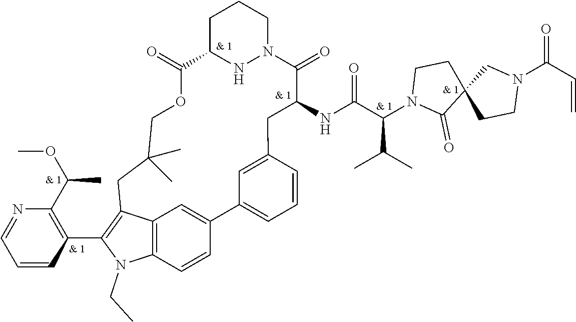 |
| B346 | 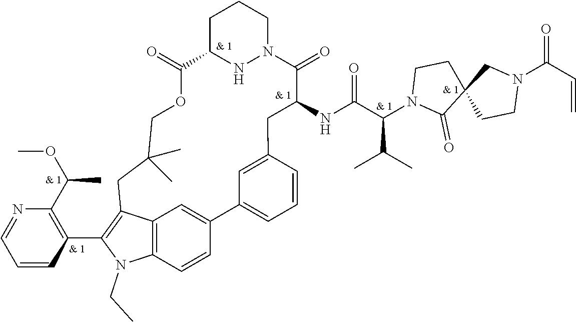 |
| B347 | 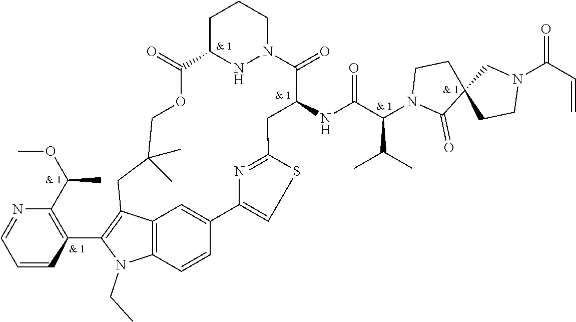 |
| B348 | 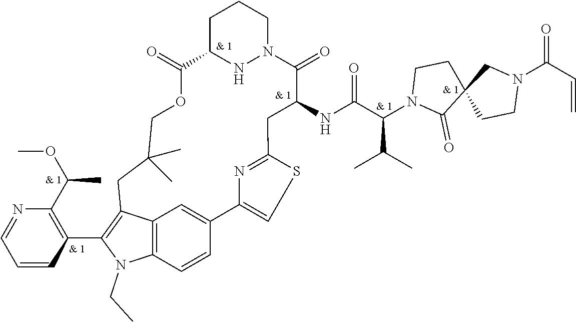 |
| B349 | 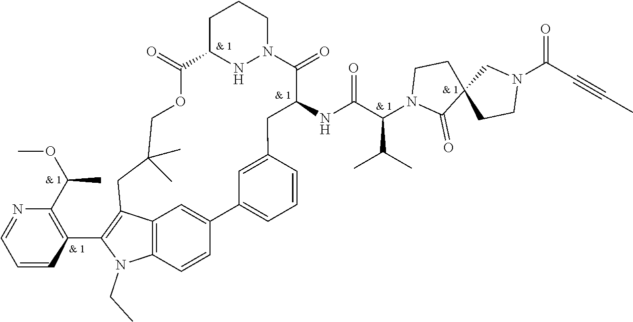 |
| B350 | 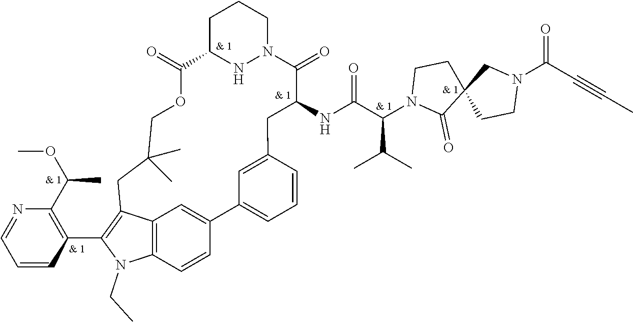 |
| B351 | 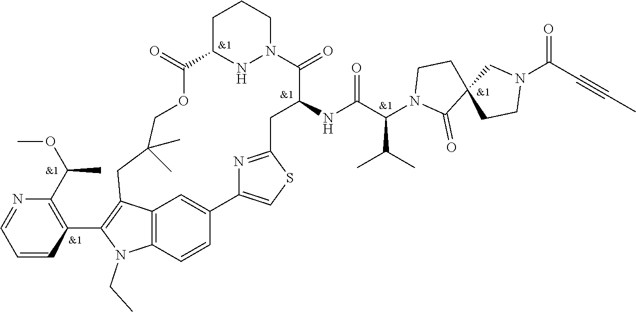 |
| B352 | 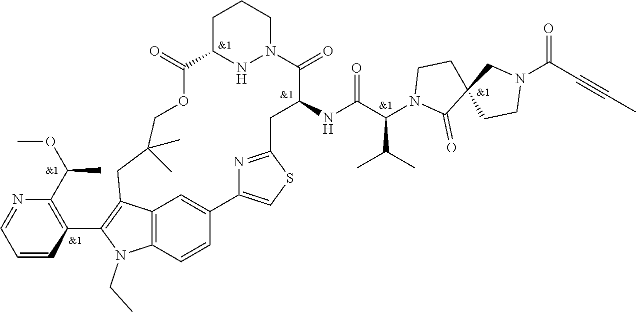 |
| B353 | 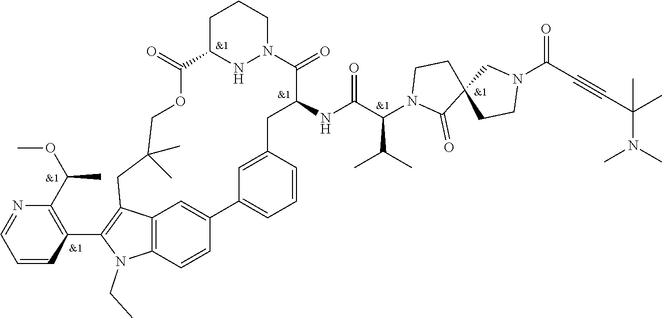 |
| B354 | 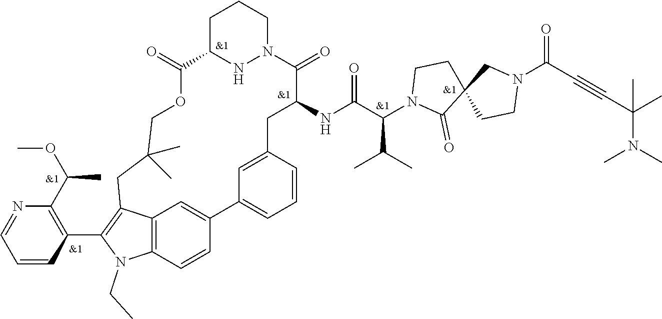 |
| B355 | 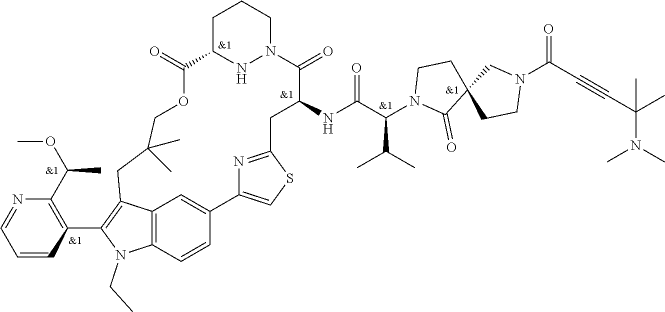 |
| B356 | 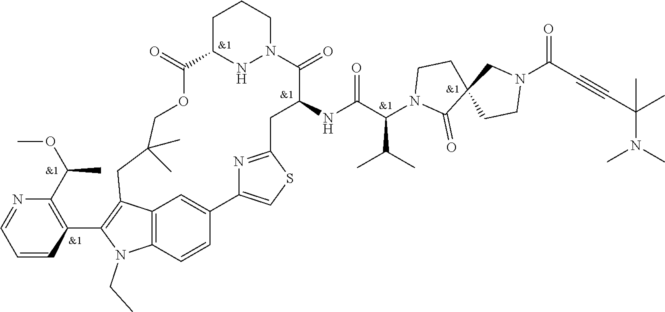 |
| B357 | 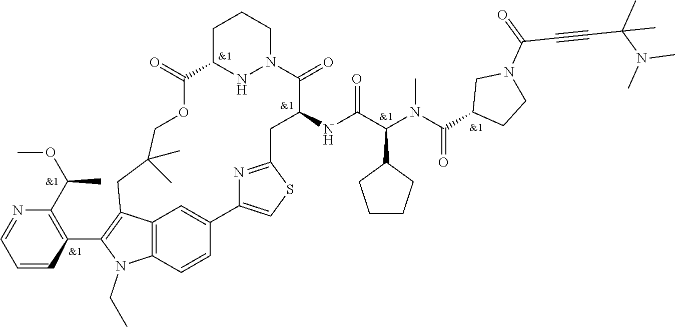 |
| B358 | 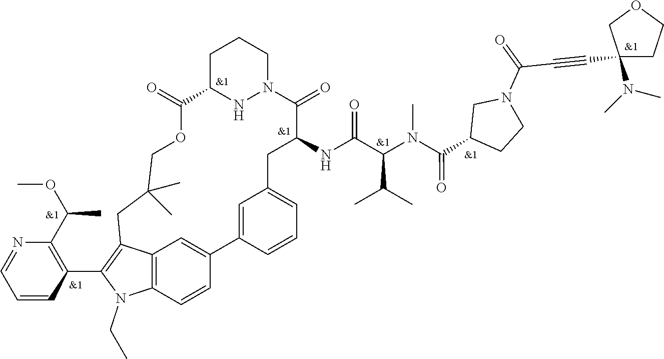 |
| B359 | 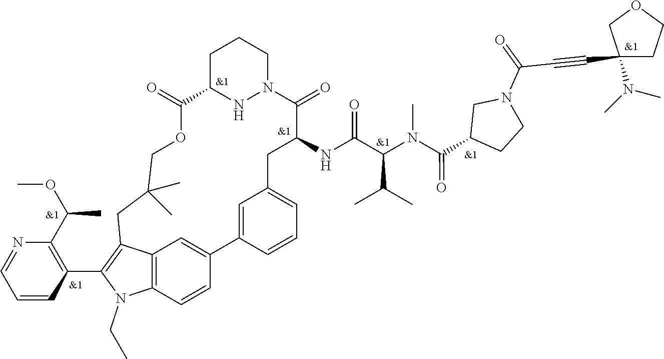 |
| B360 | 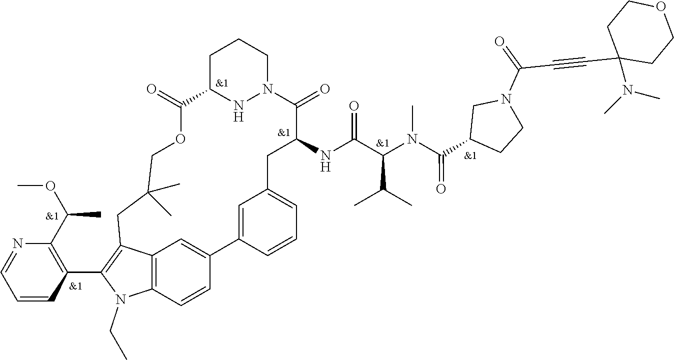 |
| B361 | 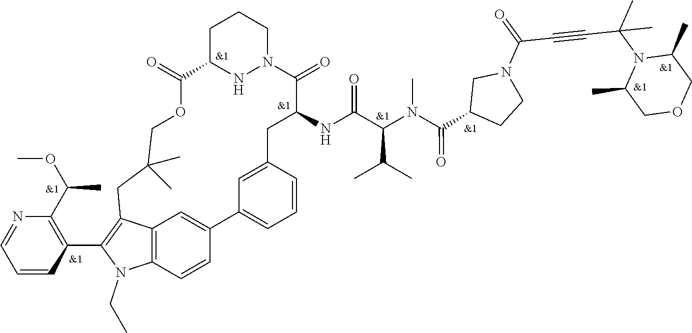 |
| B362 | 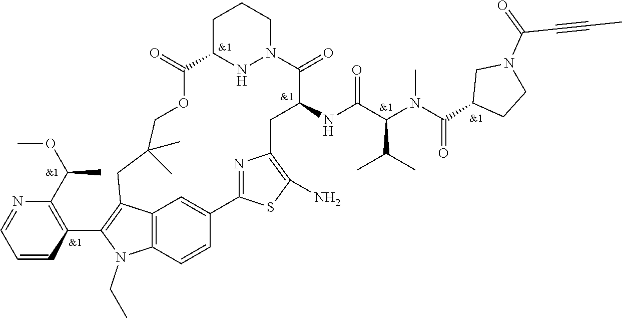 |
| B363 | 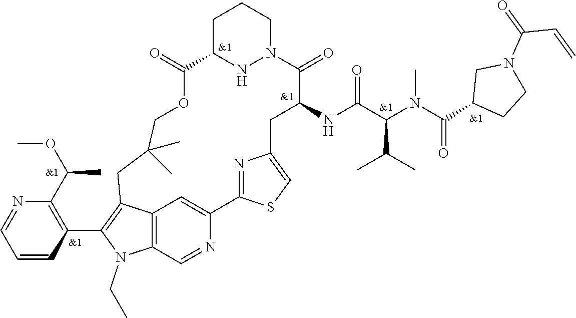 |
| B364 | 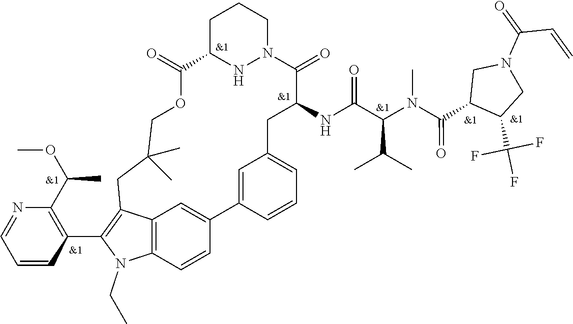 |
| B365 | 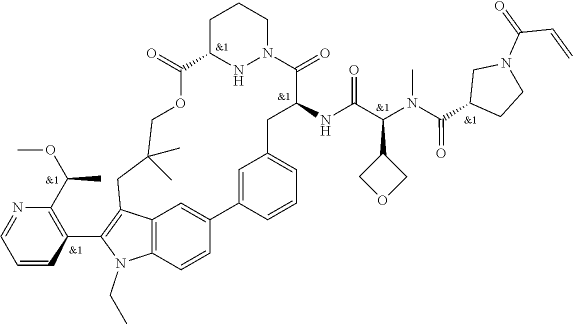 |
| B366 | 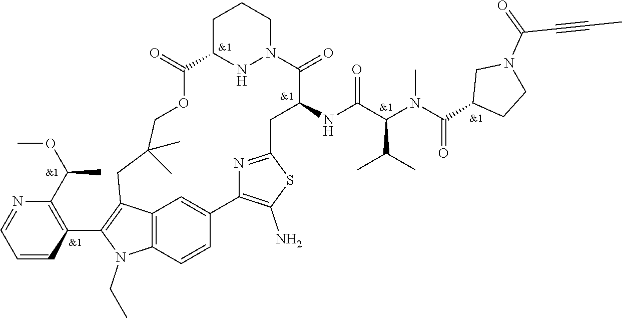 |
| B367 | 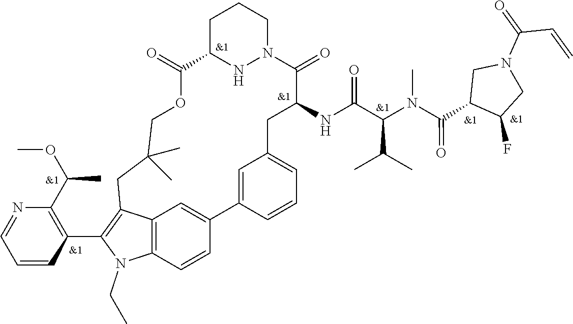 |
| B368 | 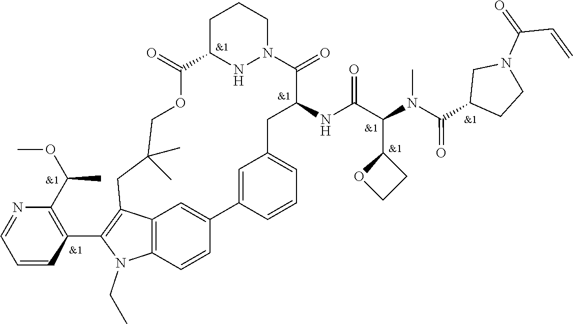 |
| B369 | 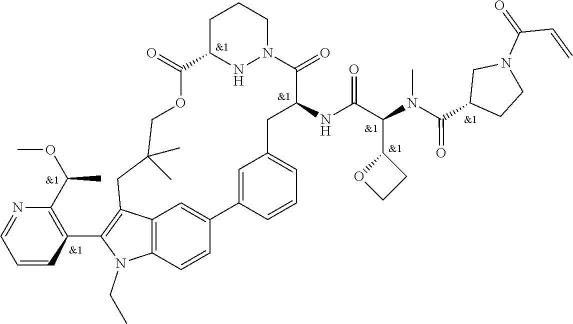 |
| B370 | 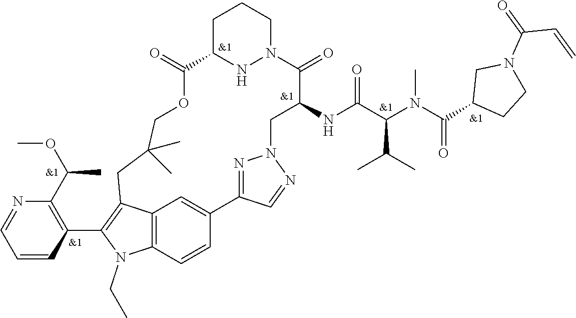 |
| B371 | 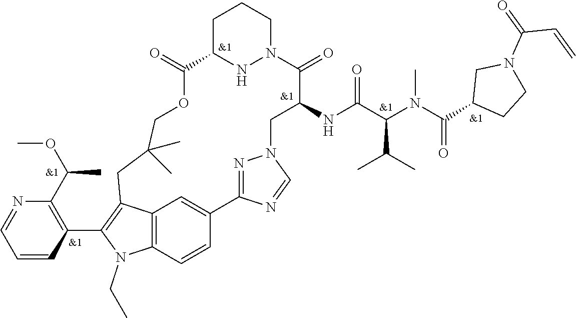 |
| B372 | 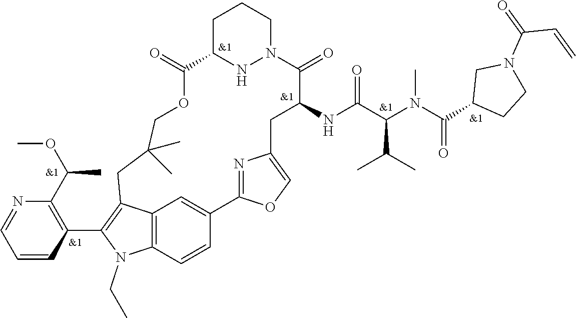 |
| B373 | 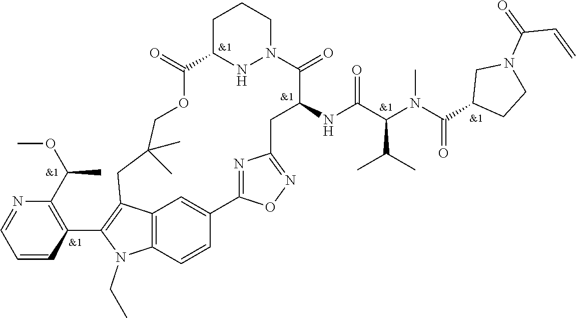 |
| B374 | 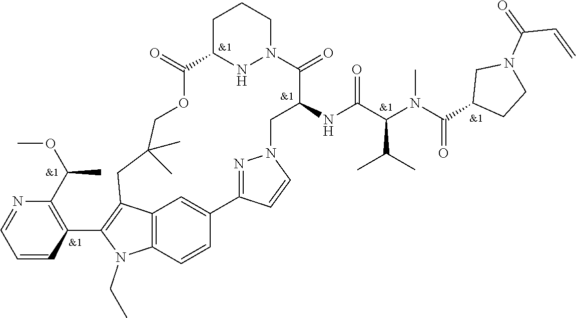 |
| B375 | 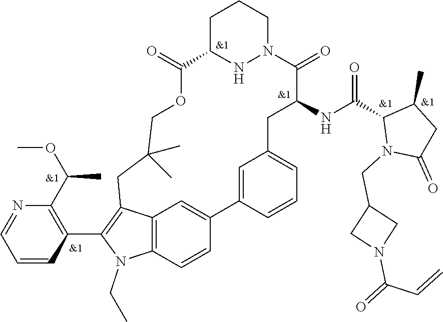 |
| B376 | 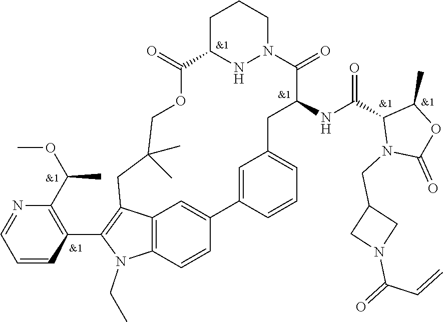 |
| B377 | 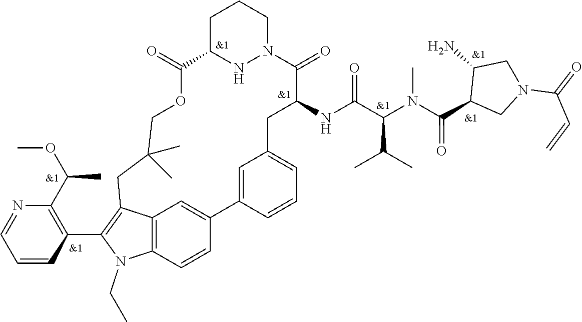 |
| B378 | 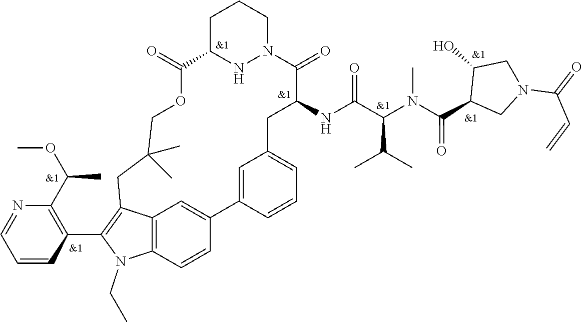 |
| B379 | 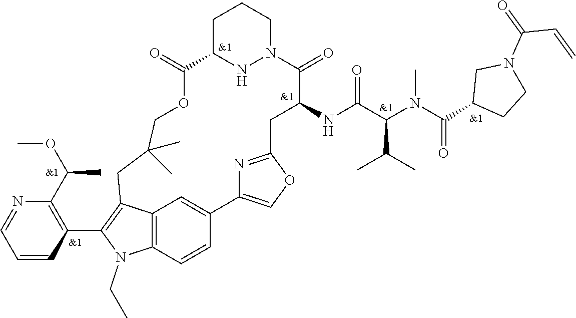 |
| B380 | 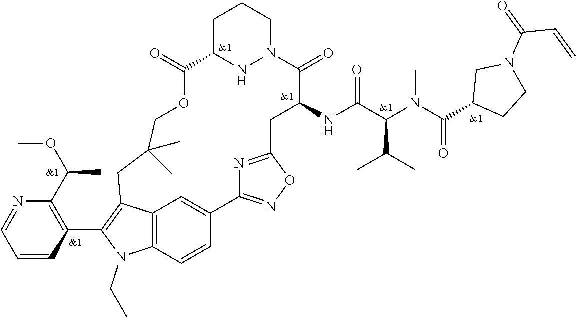 |
| B381 | 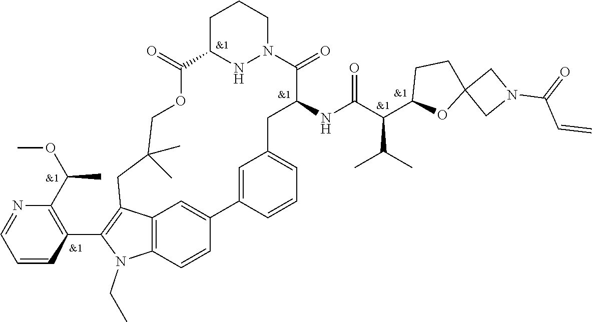 |
| B382 | 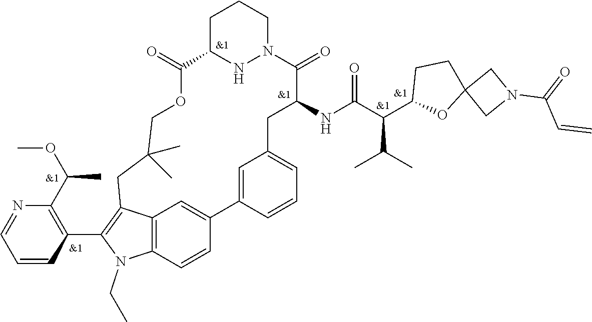 |
| B383 | 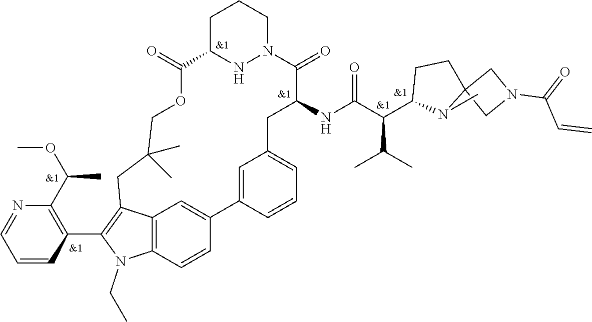 |
| B384 | 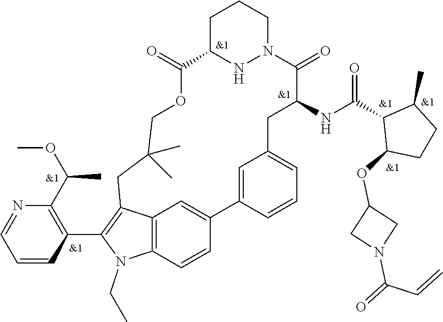 |
| B385 | 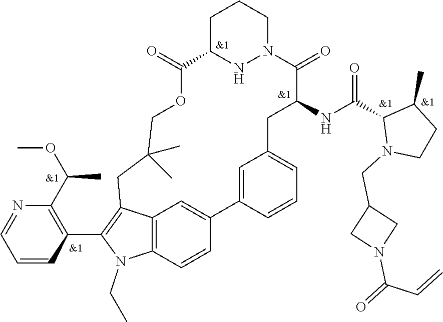 |
| B386 | 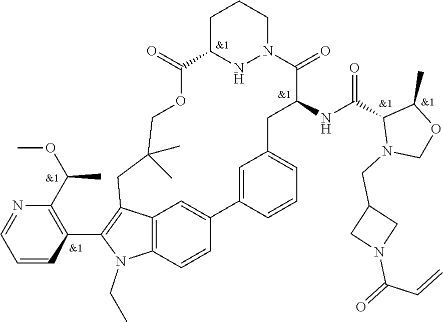 |
| B387 | 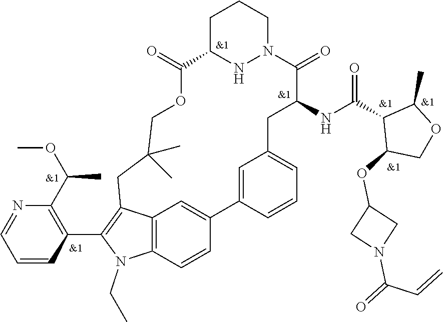 |
| B388 | 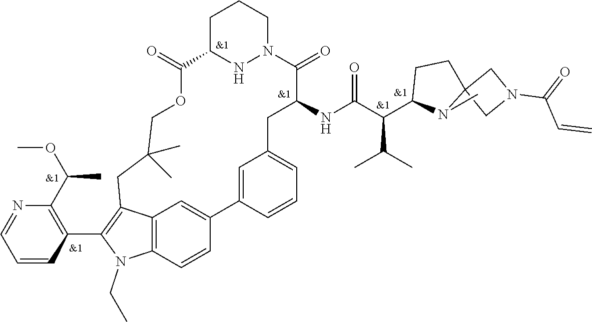 |
| B389 | 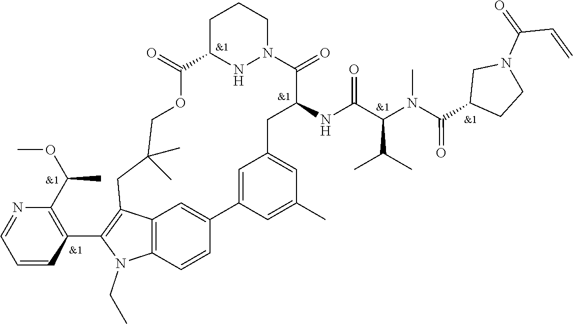 |
| B390 | 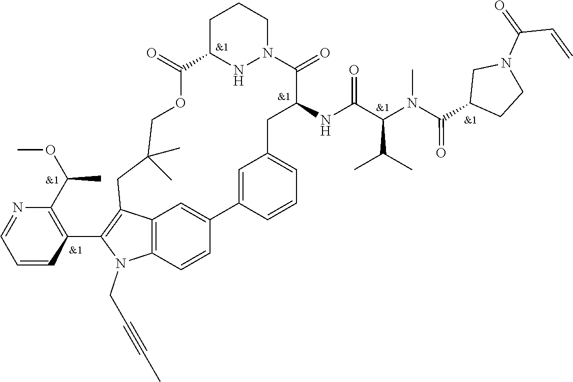 |
| B391 | 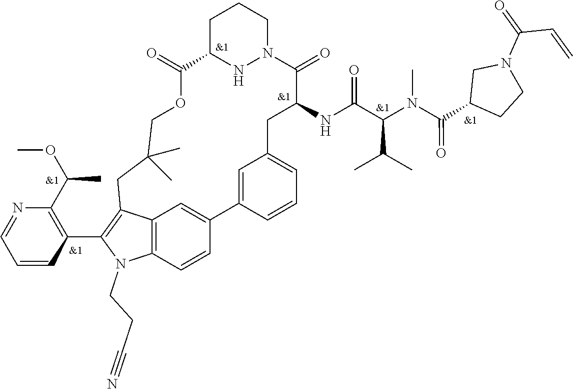 |
| B392 | 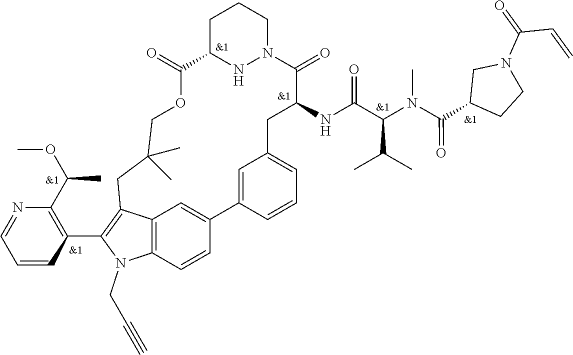 |
| B393 | 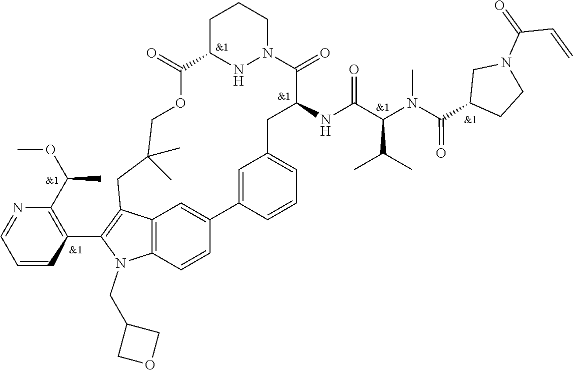 |
| B394 | 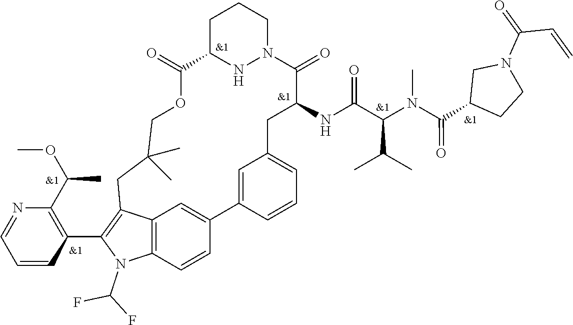 |
| B395 | 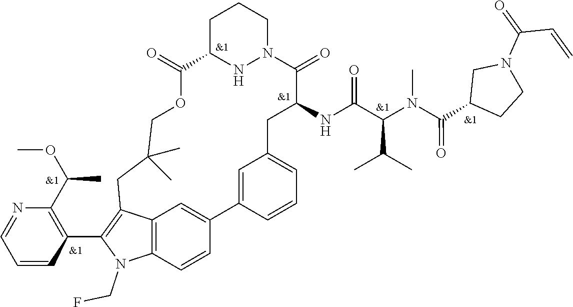 |
| B396 | 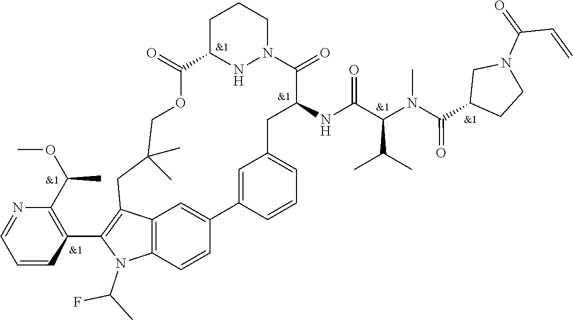 |
| B397 | 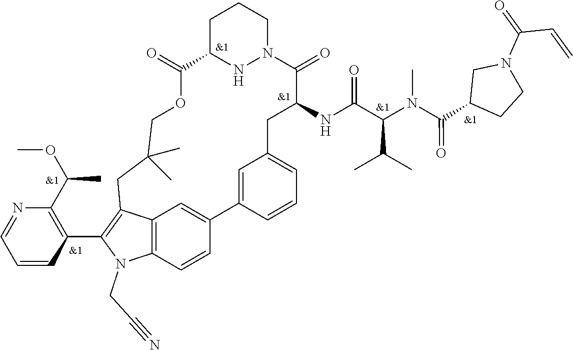 |
| B398 | 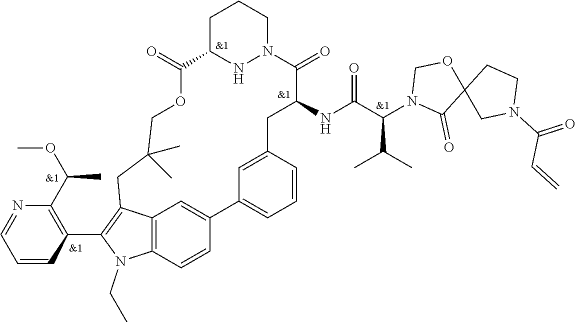 |
| B399 | 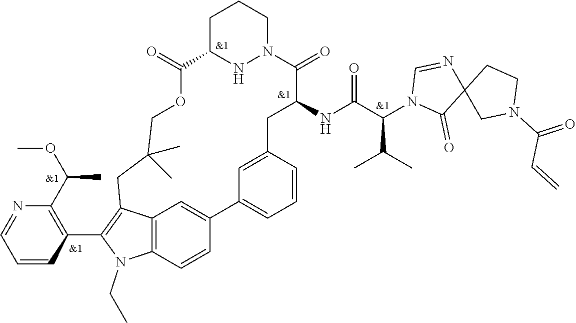 |
| B400 | 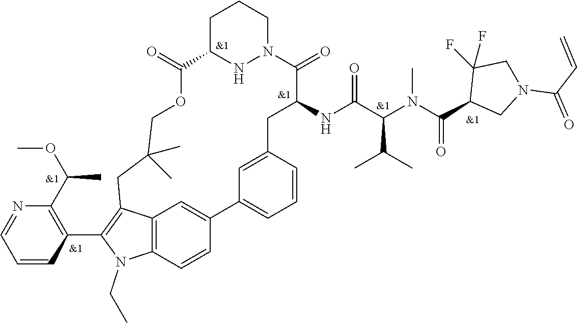 |
| B401 | 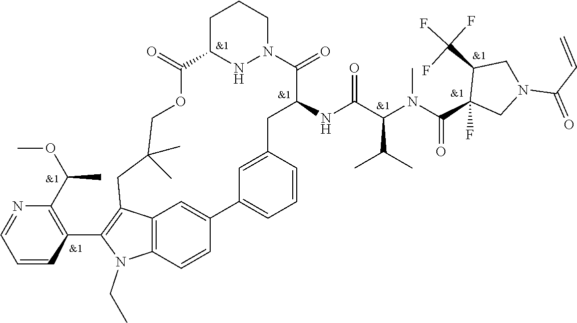 |
| B402 | 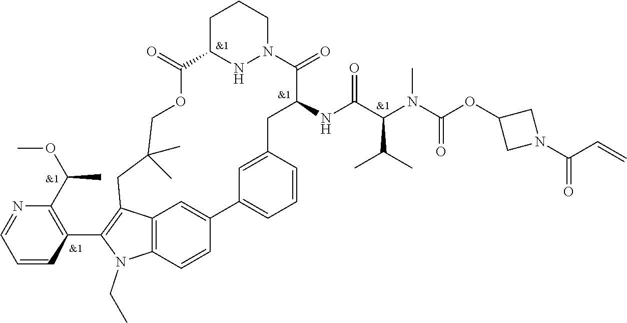 |
| B403 | 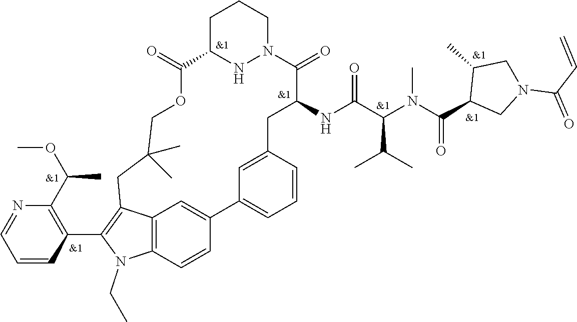 |
| B404 | 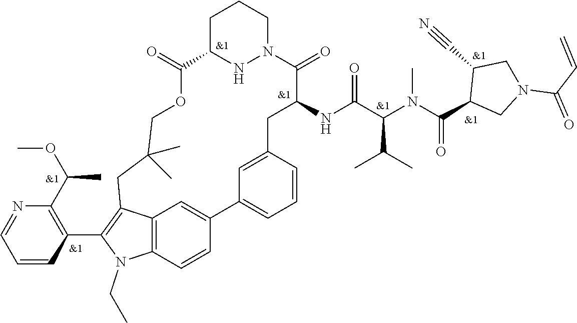 |
| B405 | 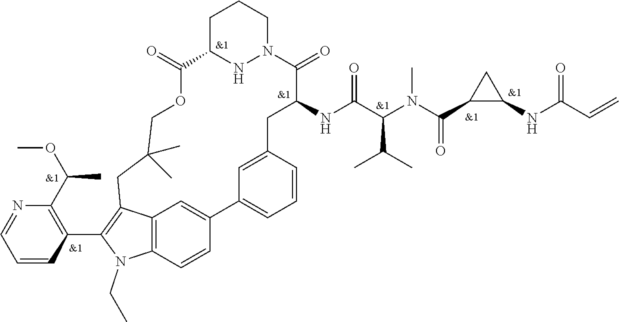 |
| B406 | 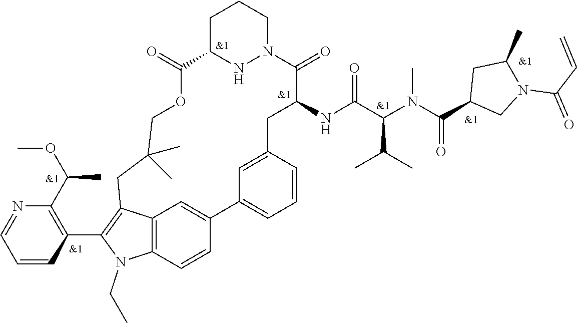 |
| B407 | 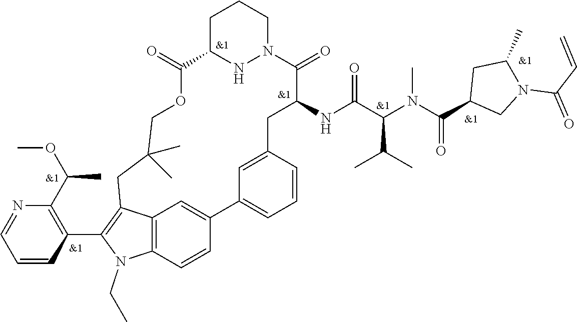 |
| B408 | 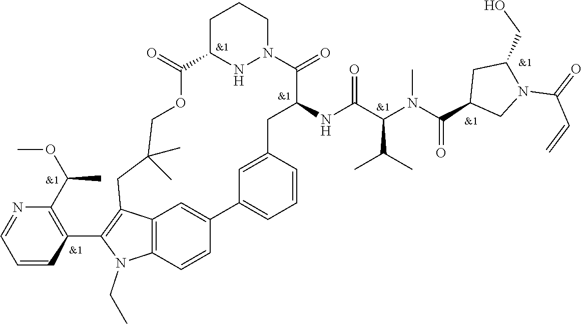 |
| B409 | 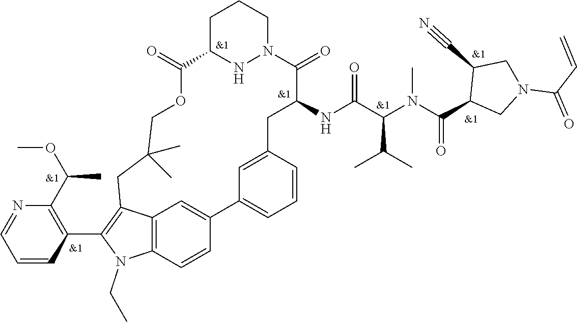 |
| B410 | 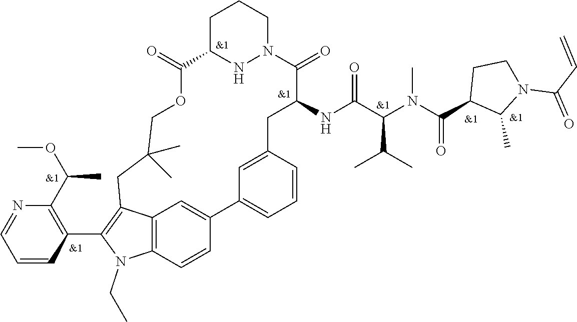 |
| B411 | 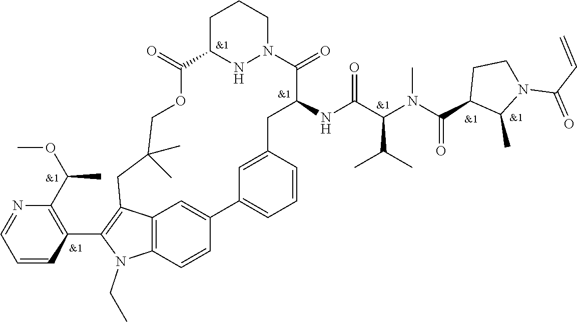 |
| B412 | 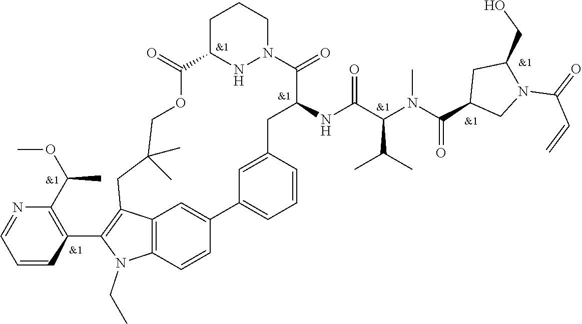 |
| B413 | 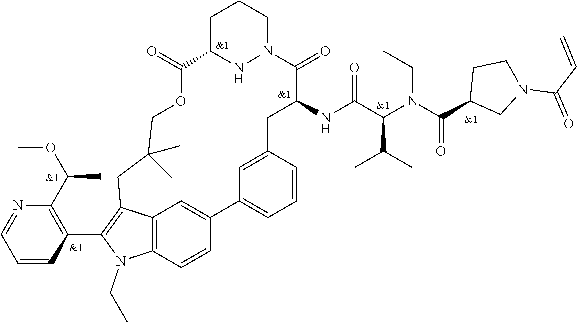 |
| B414 | 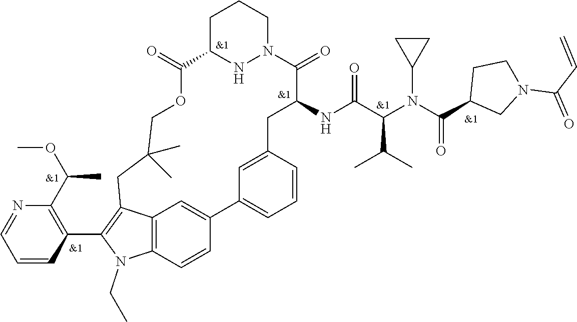 |
| B415 | 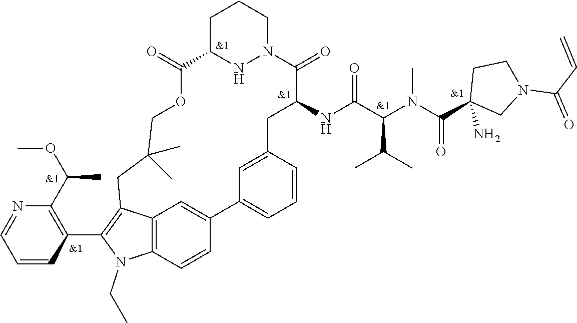 |
| B416 | 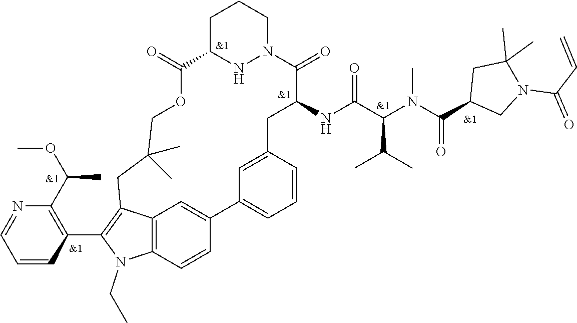 |
| B417 | 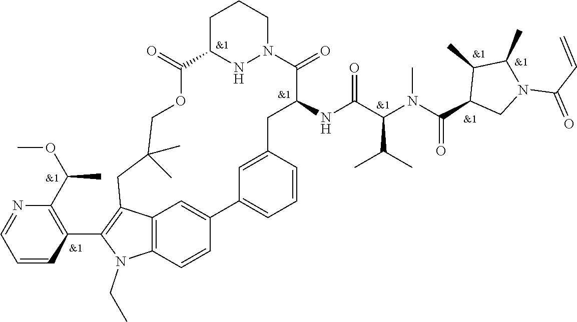 |
| B418 | 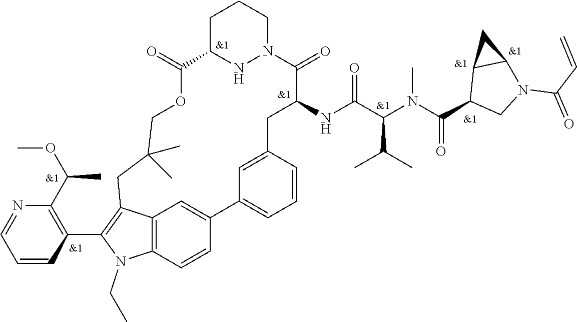 |
| B419 | 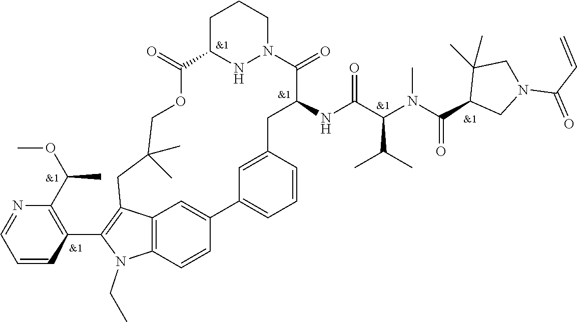 |
| B420 | 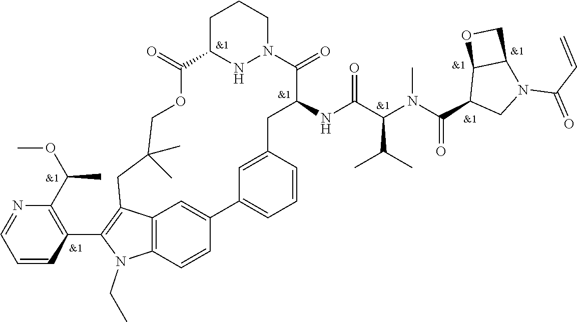 |
| B421 | 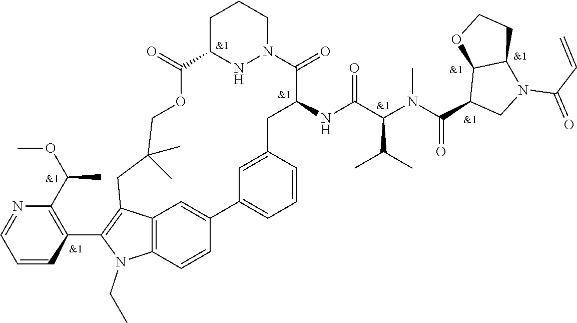 |
| B422 | 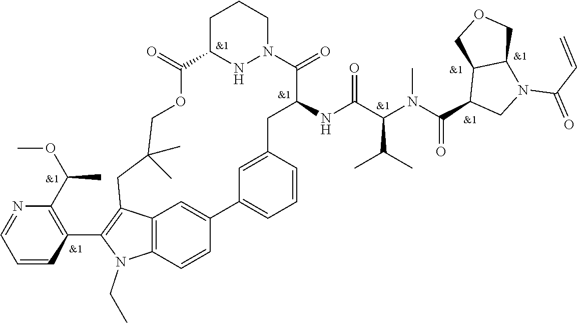 |
| B423 | 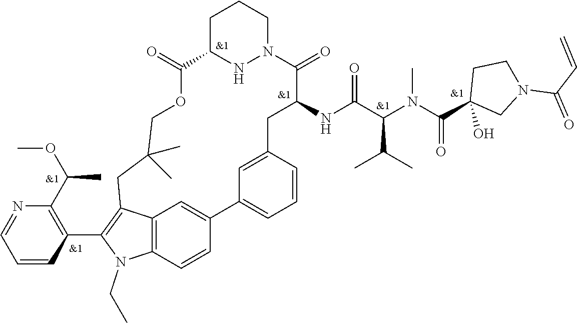 |
| B424 | 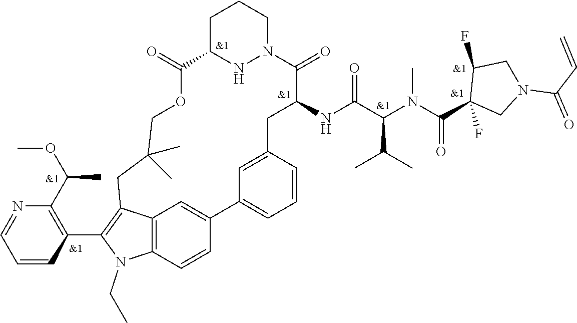 |
| B425 | 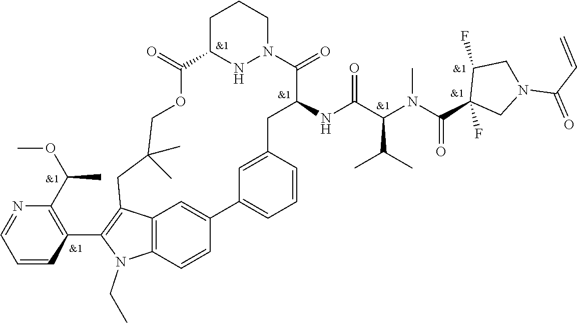 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Further provided is a conjugate, or salt thereof, comprising the structure of Formula IV:

M-L-P Formula IV
wherein L is a linker;
P is a monovalent organic moiety; and
M has the structure of Formula Va:

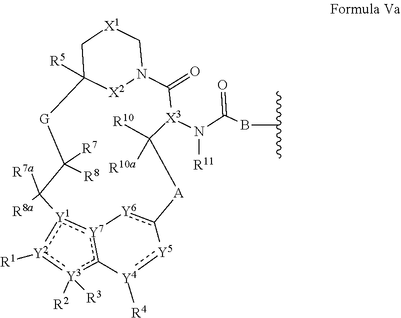

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments the conjugate, or salt thereof, comprises the structure of Formula IV:

M-L-P Formula IV
wherein L is a linker;
P is a monovalent organic moiety; and
M has the structure of Formula Vb:

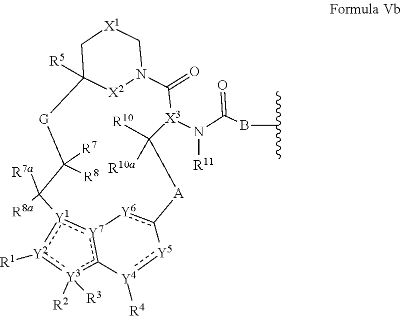

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula IV:

M-L-P Formula IV
wherein L is a linker;
P is a monovalent organic moiety; and
M has the structure of Formula Vc:

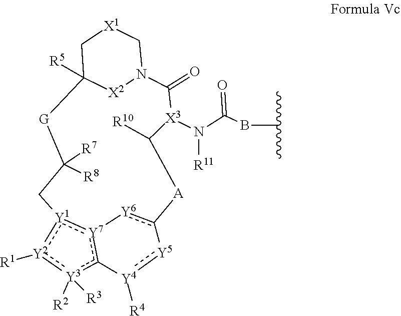

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, a compound of the present invention has the structure of of Formula IV:

M-L-P Formula IV
wherein L is a linker;
P is a monovalent organic moiety; and
M has the structure of Formula Vd:

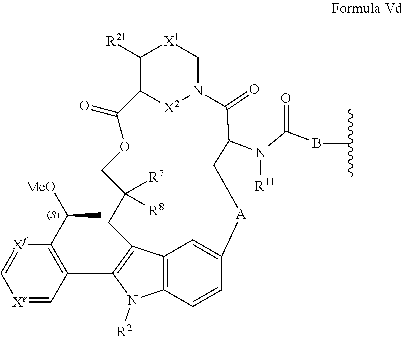

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of of Formula IV:

M-L-P Formula IV
wherein L is a linker;
P is a monovalent organic moiety; and
M has the structure of Formula Ve:

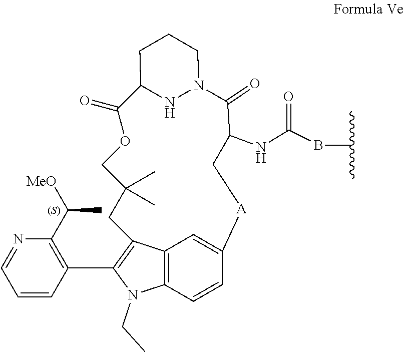

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a conjugate of the present invention, the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A².

In some embodiments of a conjugate of the present invention, the monovalent organic moiety is a protein, such as a Ras protein. In some embodiments, the Ras protein is K-Ras G12C, K-Ras G13C, H-Ras G12C, H-Ras G13C, N-Ras G12C, or N-Ras G13C. Other Ras proteins are described herein. In some embodiments, the linker is bound to the monovalent organic moiety through a bond to a sulfhydryl group of an amino acid residue of the monovalent organic moiety. In some embodiments, the linker is bound to the monovalent organic moiety through a bond to a carboxyl group of an amino acid residue of the monovalent organic moiety.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, an SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. Other additional anti-cancer therapies are described herein.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

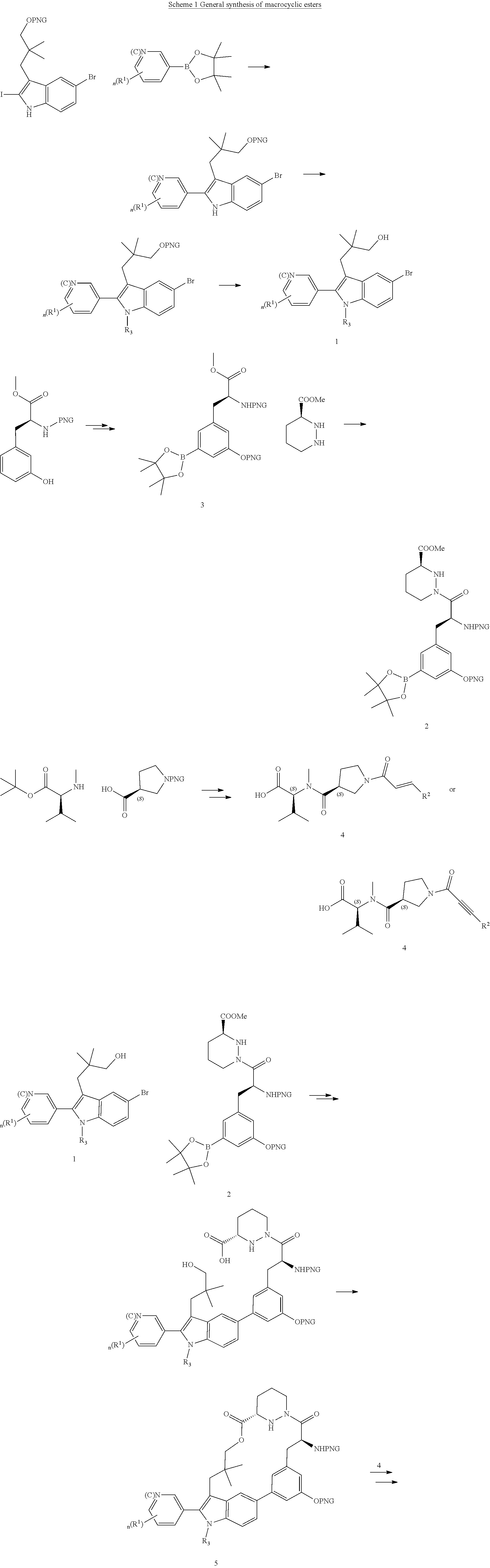

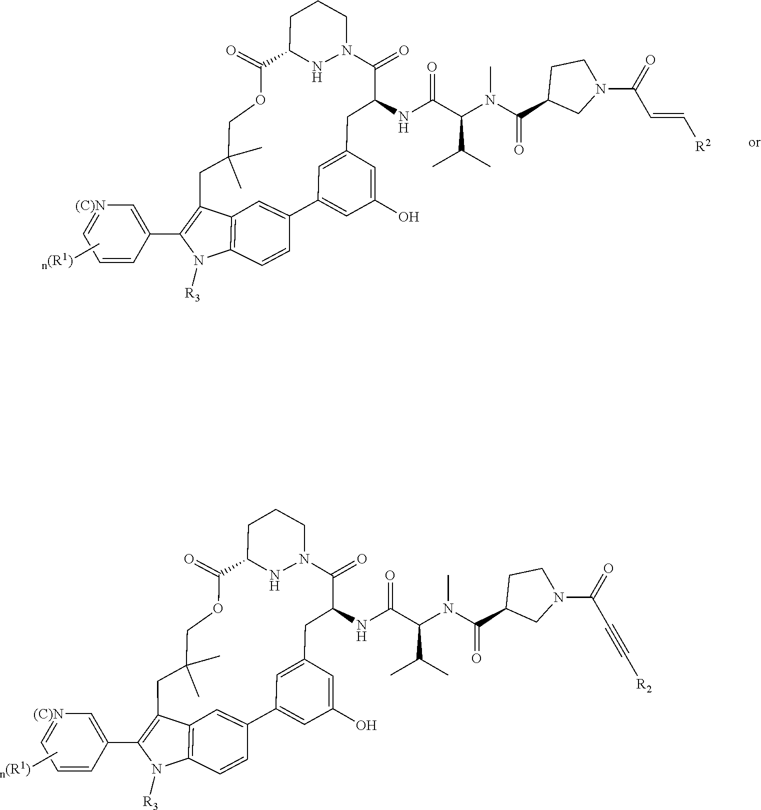

A general synthesis of macrocyclic esters is outlined in Scheme 1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl methyl(S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

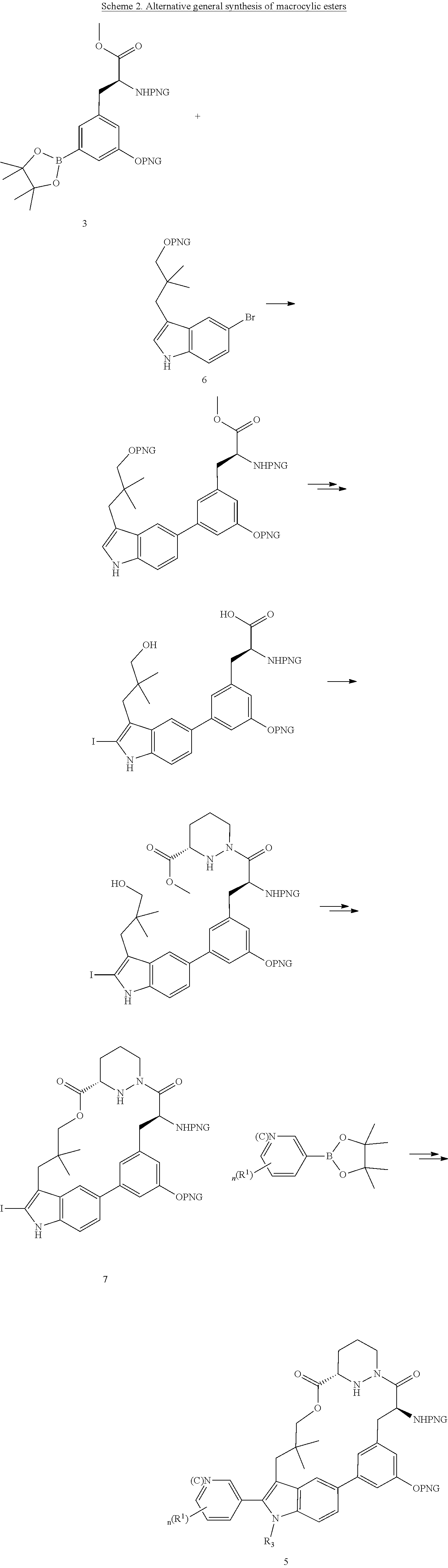

Alternatively, macrocyclic ester can be prepared as described in Scheme 2. An appropriately protected bromo-indolyl (6) coupled in the presence of a Pd catalyst with boronic ester (3), followed by iodination, deprotection, and ester hydrolysis. Subsequent coupling with methyl(S)-hexahydropyridazine-3-carboxylate, followed by hydrolysis and macrolactonization can result in iodo intermediate (7). Coupling in the presence of a Pd catalyst with an appropriately substituted boronic ester and alkyllation can yield fully protected macrocycle (5). Additional deprotection or functionalization steps are required to produce the final compound.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in the Example section herein.

Compounds of Table 1 herein were prepared using methods disclosed herein or were prepared using methods disclosed herein combined with the knowledge of one of skill in the art. Compounds of Table 2 may be prepared using methods disclosed herein or may be prepared using methods disclosed herein combined with the knowledge of one of skill in the art.

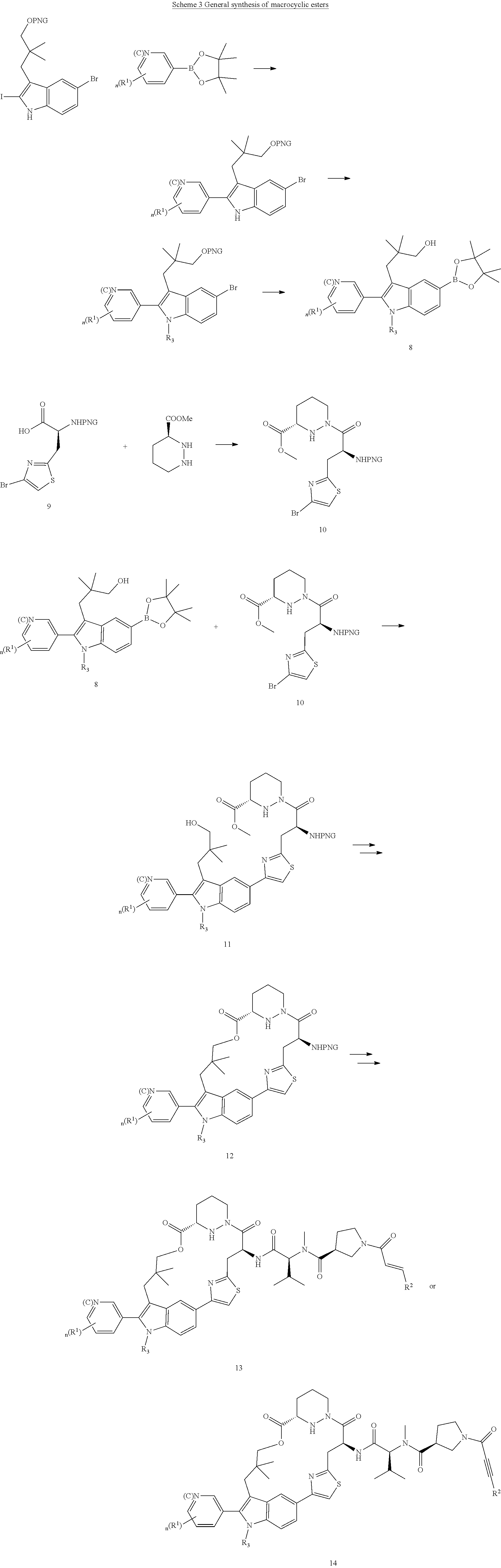

An alternative general synthesis of macrocyclic esters is outlined in Scheme 3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl) propanoyl) hexahydropyridazine-3-carboxylate (10) can be prepared via coupling of(S)-2-amino-3-(4-bromothiazol-2-yl) propanoic acid (9) with methyl(S) -hexahydropyridazine-3-carboxylate.

The final macrocyclic esters can be made by coupling of Methyl-amino-3-(4-bromothiazol-2-yl) propanoyl) hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

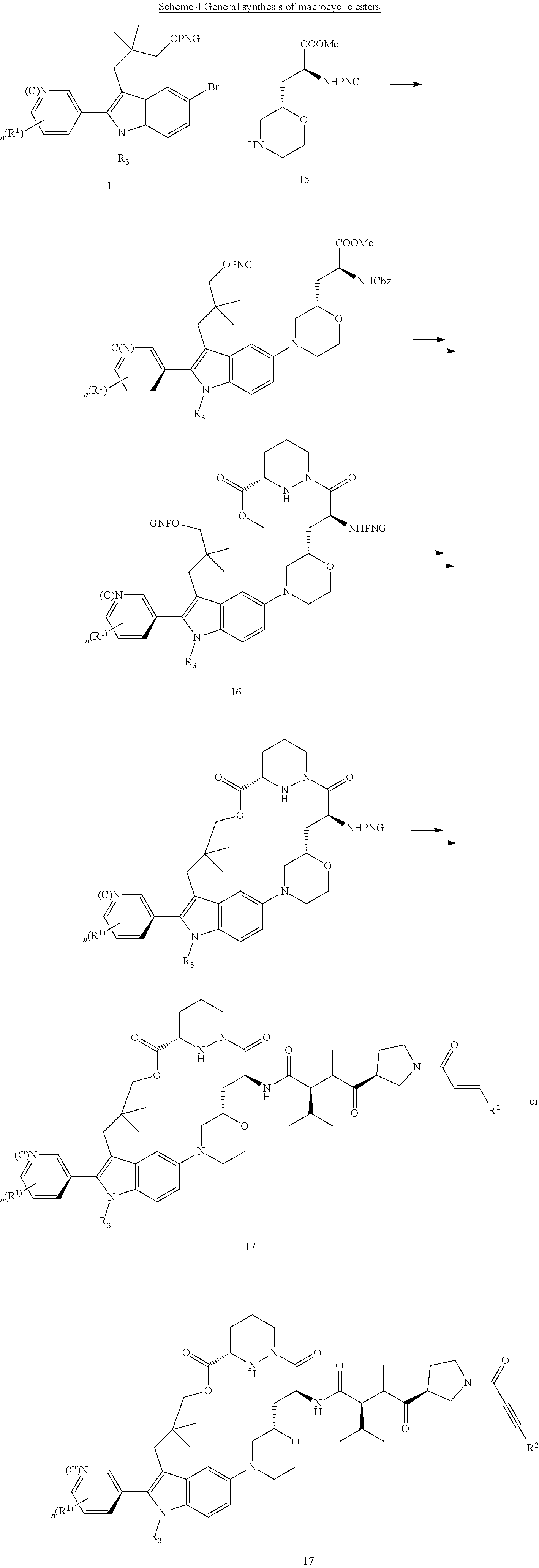

An alternative general synthesis of macrocyclic esters is outlined in Scheme 4. An appropriately substituted morpholine or an alternative herecyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

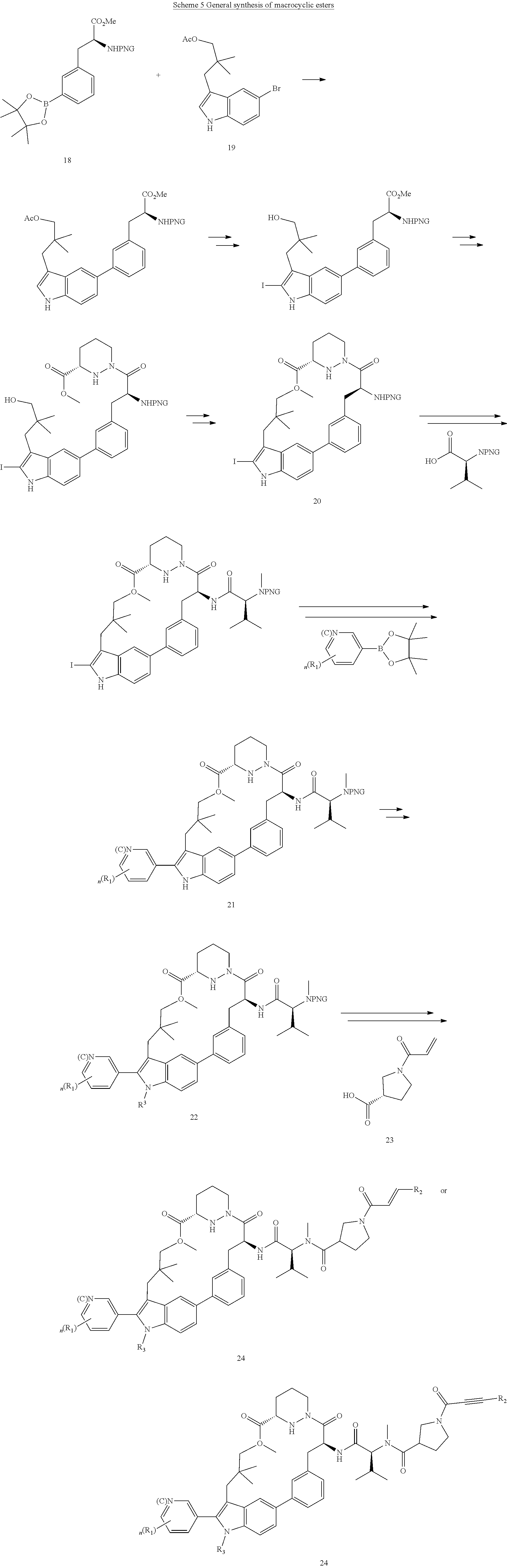

An alternative general synthesis of macrocyclic esters is outlined in Scheme 5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclization steps. Subsequent coupling with an appropriately substituted protected aminoacid followed by palladium mediated coupling yields intermediate 21. Additional deprotection and derivatization steps, including alkylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions.

Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

### Numbered Embodiments

[1] A compound, or pharmaceutically acceptable salt thereof, having the structure of Formula I:

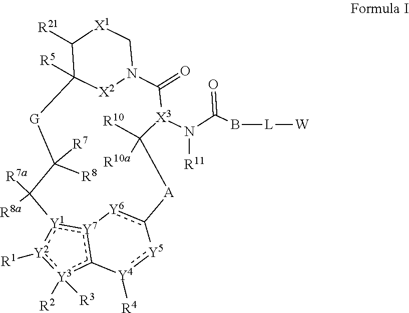

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is H or C₁-C₃ alkyl.
[2] The compound, or pharmaceutically acceptable salt thereof, of paragraph [1], wherein G is optionally substituted C₁-C₄ heteroalkylene.
[3] The compound, or pharmaceutically acceptable salt thereof, of paragraph [1] or [2], wherein the compound has the structure of Formula Ic:

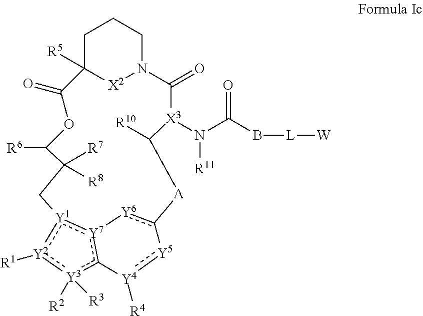

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.
[4] The compound, or pharmaceutically acceptable salt thereof, of any one of paragraphs [1] to [3], wherein X² is NH.
[5] The compound, or pharmaceutically acceptable salt thereof, of any one of paragraphs [1] to [4], wherein X³ is CH.
[6] The compound, or pharmaceutically acceptable salt thereof, of any one of paragraphs [1] to [5], wherein R¹¹ is hydrogen.
[7] The compound, or pharmaceutically acceptable salt thereof, of any one of paragraphs [1] to [5], wherein R¹¹ is C₁-C₃ alkyl.
[8] The compound, or pharmaceutically acceptable salt thereof, of paragraph [7], wherein R¹¹ is methyl.
[9] The compound, or pharmaceutically acceptable salt thereof, of any one of paragraphs [1] to [6], wherein the compound has the structure of Formula Id:

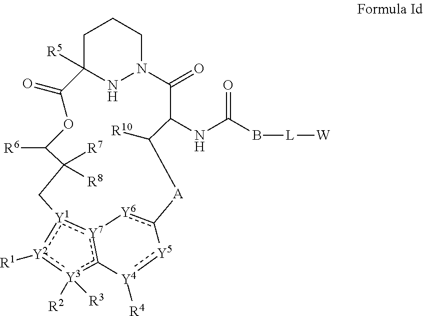

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′;

C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and

R¹⁰ is hydrogen, hydroxy, C₁-C₃ alkoxy, or C₁-C₃ alkyl.
[10] The compound, or pharmaceutically acceptable salt thereof, of any one of paragraphs [1] to [9] wherein X¹ is optionally substituted C₁-C₂ alkylene.
[11] The compound, or pharmaceutically acceptable salt thereof, of paragraph [10], wherein X¹ is methylene.
[12] The compound, or pharmaceutically acceptable salt thereof, of any one of paragraphs [1] to [11], wherein R⁵ is hydrogen.
[13] The compound, or pharmaceutically acceptable salt thereof, of any one of paragraphs [1] to [11], wherein R⁵ is C₁-C₄ alkyl optionally substituted with halogen.
[14] The compound, or pharmaceutically acceptable salt thereof, of paragraph [13], wherein R⁵ is methyl.
[15] The compound, or pharmaceutically acceptable salt thereof, of any one of paragraphs [1] to [14], wherein Y⁴ is C.
[16] The compound, or pharmaceutically acceptable salt thereof, of any one of paragraphs [1] to [15], wherein R⁴ is hydrogen.
[17] The compound, or pharmaceutically acceptable salt thereof, of any one of paragraphs [1] to [16], wherein Y⁵ is CH.
[18] The compound, or pharmaceutically acceptable salt thereof, of any one of paragraphs [1] to [17], wherein Y⁶ is CH.
[19] The compound, or pharmaceutically acceptable salt thereof, of any one of paragraphs [1] to [18], wherein Y¹ is C.
[20] The compound, or pharmaceutically acceptable salt thereof, of any one of paragraphs [1] to [19] wherein Y² is C.
[21] The compound, or pharmaceutically acceptable salt thereof, of any one of paragraphs [1] to [20] wherein Y³ is N.
[22] The compound, or pharmaceutically acceptable salt thereof, of any one of paragraphs [1] to [21], wherein R³ is absent.
[23] The compound, or pharmaceutically acceptable salt thereof, of any one of paragraphs [1] to [22], wherein Y⁷ is C.
[24] The compound, or pharmaceutically acceptable salt thereof, of any one of paragraphs [1] to [6] or [9] to [23], wherein the compound has the structure of Formula I:

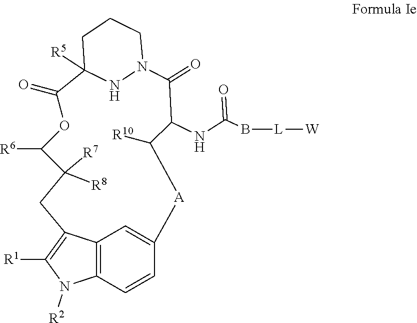

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.
[25] The compound, or pharmaceutically acceptable salt thereof, of any one of paragraphs [3] to [24], wherein R⁶ is hydrogen.
[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.
[27] The compound, or pharmaceutically acceptable salt thereof, of paragraph [26], wherein R² is optionally substituted C₁-C₆ alkyl.
[28] The compound, or pharmaceutically acceptable salt thereof, of paragraph [27], wherein R² is ethyl.
[29] The compound, or pharmaceutically acceptable salt thereof, of any one of paragraphs [1] to [28], wherein R⁷ is optionally substituted C₁-C₃ alkyl.
[30] The compound, or pharmaceutically acceptable salt thereof, of paragraph 29, wherein R⁷ is C₁-C₃ alkyl.
[31] The compound, or pharmaceutically acceptable salt thereof, of any one of paragraphs [1] to 30, wherein R⁸ is optionally substituted C₁-C₃ alkyl.
[32] The compound, or pharmaceutically acceptable salt thereof, of paragraph [31], wherein R⁸ is C₁-C₃ alkyl.
[33] The compound, or pharmaceutically acceptable salt thereof, of any one of paragraphs [1] to [32], wherein the compound has the structure of Formula If:

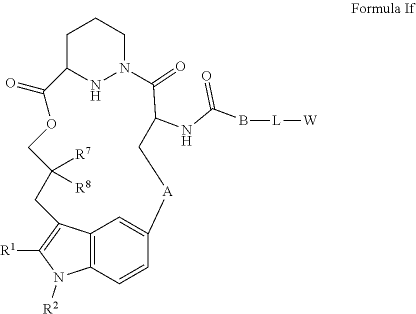

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[34] The compound, or pharmaceutically acceptable salt thereof, of any one of paragraphs [1] to [33], wherein R¹ is optionally substituted 6 to 10-membered aryl, optionally substituted 3 to 6-membered cycloalkenyl, or optionally substituted 5 to 10-membered heteroaryl.
[35] The compound, or pharmaceutically acceptable salt thereof, of paragraph [34], wherein R¹ is optionally substituted 6-membered aryl, optionally substituted 6-membered cycloalkenyl, or optionally substituted 6-membered heteroaryl.
[36] The compound, or pharmaceutically acceptable salt thereof, of paragraph [35], wherein R¹ is

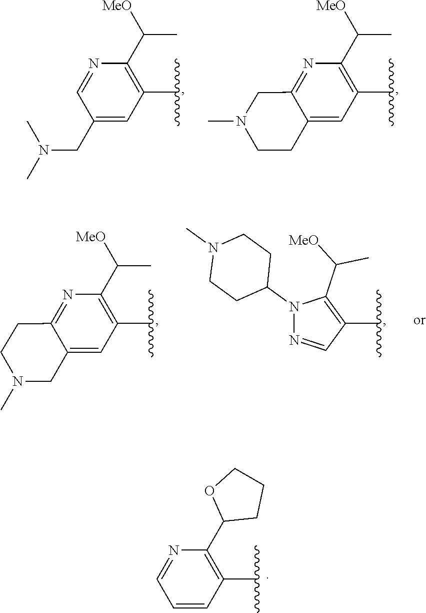

[37] The compound, or pharmaceutically acceptable salt thereof, of paragraph [36], wherein R¹ is

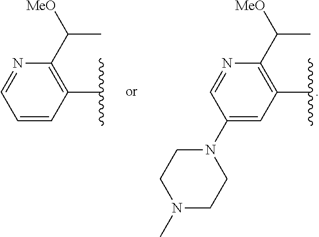 [SMILES: *c1cccnc1C(C)OC ; *c1cc(N2CCN(C)CC2)cnc1C(C)OC]

[38] The compound, or pharmaceutically acceptable salt thereof, of any one of paragraphs [1] to [37], wherein the compound has the structure of Formula Ig:

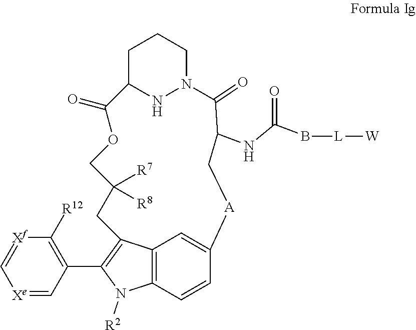

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} and X^{f} are, independently, N or CH; and
R¹² is optionally substituted C₁-C₆ alkyl, optionally substituted C₁-C₆ heteroalkyl, or optionally substituted 3 to 7-membered heterocycloalkyl.
[39] The compound, or pharmaceutically acceptable salt thereof, of paragraph [38], wherein X^{e} is N and X^{f} is CH.
[40] The compound, or pharmaceutically acceptable salt thereof, of paragraph [38], wherein X^{e} is CH and X^{f} is N.
[41] The compound, or pharmaceutically acceptable salt thereof, of any one of paragraphs [38] to [40], wherein R¹² is optionally substituted C₁-C₆ heteroalkyl.
[42] The compound, or pharmaceutically acceptable salt thereof, of any one of paragraphs [38] to [41], wherein R¹² is

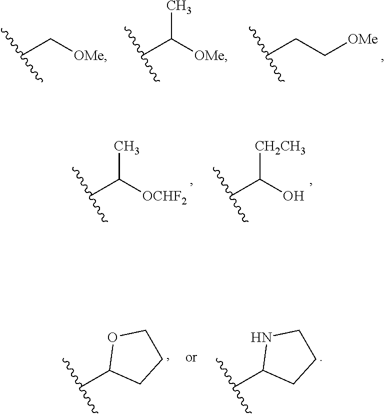

[43] The compound, or pharmaceutically acceptable salt thereof, of paragraph [1] or [2], wherein the compound has the structure of Formula VI:

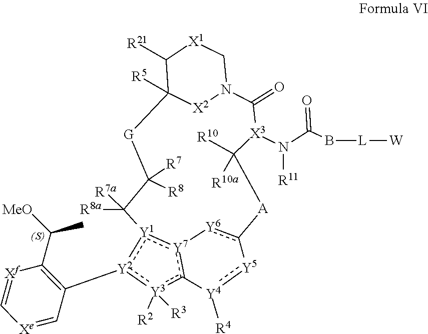

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.
[44] The compound, or pharmaceutically acceptable salt thereof, of paragraph [43], wherein the compound has the structure of Formula VIa:

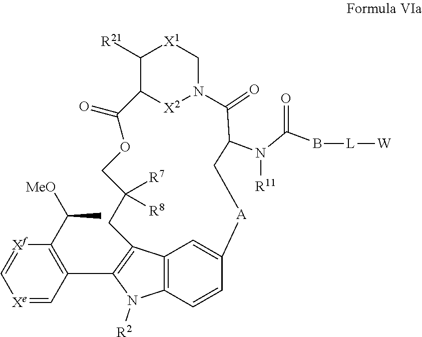

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.
[45] The compound, or pharmaceutically acceptable salt thereof, of paragraph [43] or [44], wherein the compound has the structure of Formula VIb:

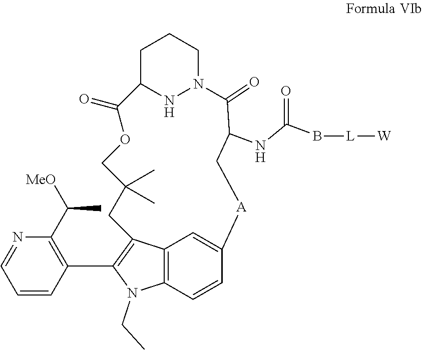

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone.
[46] The compound, or pharmaceutically acceptable salt thereof, of any one of paragraphs [1] to [45], wherein A is optionally substituted 6-membered arylene.
[47] The compound, or pharmaceutically acceptable salt thereof, of paragraph [46], wherein A has the structure:

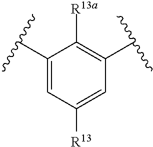

wherein R¹³ is hydrogen, halo, hydroxy, amino, optionally substituted C₁-C₆ alkyl, or optionally substituted C₁-C₆ heteroalkyl; and
R^{13a} is hydrogen or halo.
[48] The compound, or pharmaceutically acceptable salt thereof, of paragraph [47], wherein R¹³ and R^{13a} are each hydrogen.
[49] The compound, or pharmaceutically acceptable salt thereof, of paragraph [47], wherein R¹³ is hydroxy, methyl, fluoro, or difluoromethyl.
[50] The compound, or pharmaceutically acceptable salt thereof, of any one of paragraphs [1] to [45], wherein A is optionally substituted 5 to 6-membered heteroarylene.
[51] The compound, or pharmaceutically acceptable salt thereof, of paragraph [50], wherein A is:

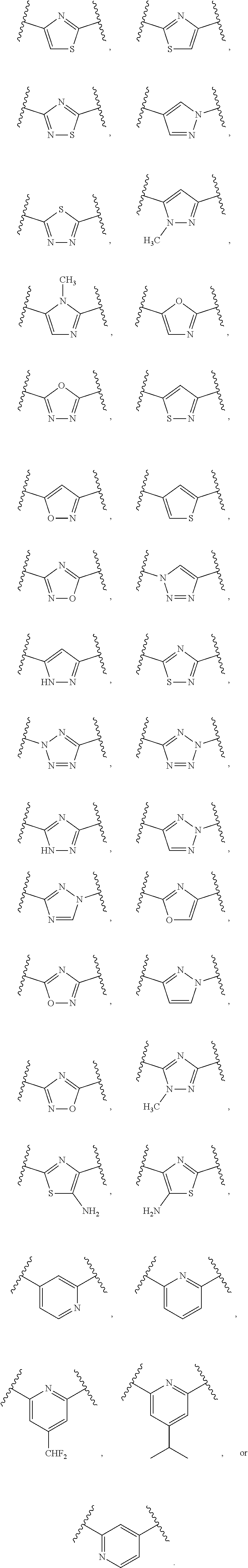

[52] The compound, or pharmaceutically acceptable salt thereof, of any one of paragraphs [1] to [45], wherein A is optionally substituted C₁-C₄ heteroalkylene.
[53] The compound, or pharmaceutically acceptable salt thereof, of paragraph [52], wherein A is:

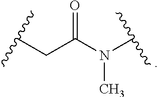 [SMILES: *CC(=O)N(*)C]

[54] The compound, or pharmaceutically acceptable salt thereof, of any one of paragraphs [1] to [45], wherein A is optionally substituted 3 to 6-membered heterocycloalkylene.
[55] The compound, or pharmaceutically acceptable salt thereof, of paragraph [54], wherein A is:

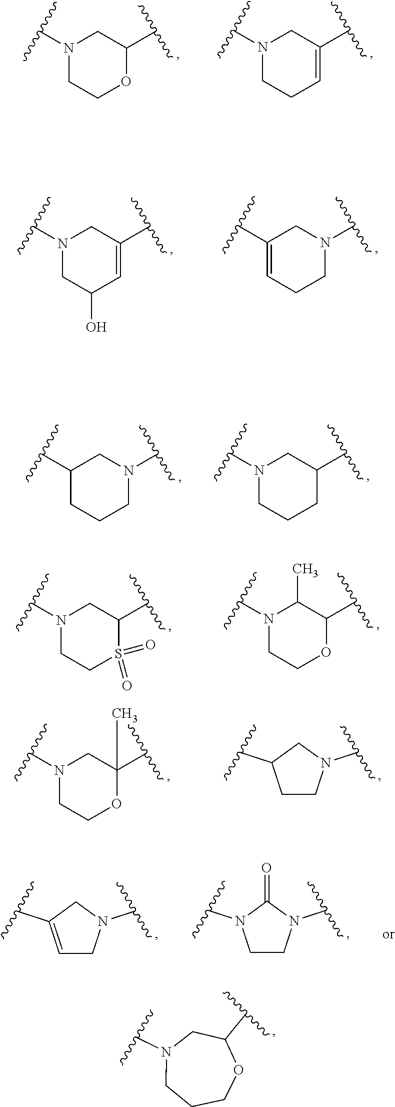

[56] The compound, or pharmaceutically acceptable salt thereof, of paragraph [55], wherein A is

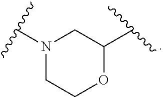 [SMILES: *C1CN(*)CCO1]

[57] The compound, or pharmaceutically acceptable salt thereof, of any one of paragraphs [1] to [56], wherein B is —CHR⁹—.
[58] The compound, or pharmaceutically acceptable salt thereof, of paragraph [57], wherein R⁹ is F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[59] The compound, or pharmaceutically acceptable salt thereof, of paragraph [58], wherein R⁹ is:

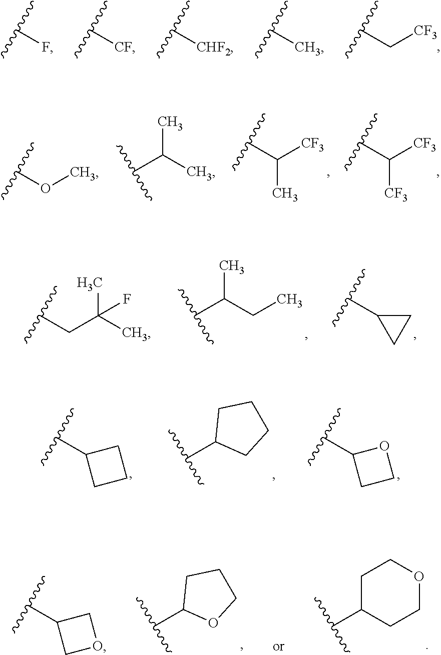

[60] The compound, or pharmaceutically acceptable salt thereof, of paragraph [59], wherein R⁹ is:

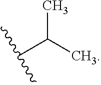 [SMILES: *C(C)C]

[61] The compound, or pharmaceutically acceptable salt thereof, of any one of paragraphs [1] to [56], wherein B is optionally substituted 6-membered arylene.
[62] The compound, or pharmaceutically acceptable salt thereof, of paragraph [61], wherein B is 6-membered arylene.
[63] The compound, or pharmaceutically acceptable salt thereof, of paragraph [61], wherein B is:

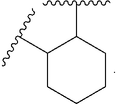 [SMILES: *C1CCCCC1*]

[64] The compound, or pharmaceutically acceptable salt thereof, of any one of paragraphs [1] to [63], wherein R⁷ is methyl.
[65] The compound, or pharmaceutically acceptable salt thereof, of any one of paragraphs [1] to [64], wherein R⁸ is methyl.
[66] The compound, or pharmaceutically acceptable salt thereof, of any one of paragraphs [1] to [65], wherein the linker is the structure of Formula II:

A¹-(B¹)^{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².
[67] The compound, or a pharmaceutically acceptable salt thereof, of any one of paragraphs [1] to [66], wherein the linker is acyclic.
[68] The compound, or a pharmaceutically acceptable salt thereof, of paragraph [67], wherein the linker has the structure of Formula IIa:

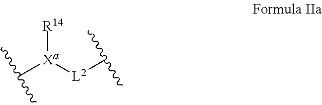

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present.
[69] The compound, or a pharmaceutically acceptable salt thereof, of paragraph [68], wherein the linker has the structure:

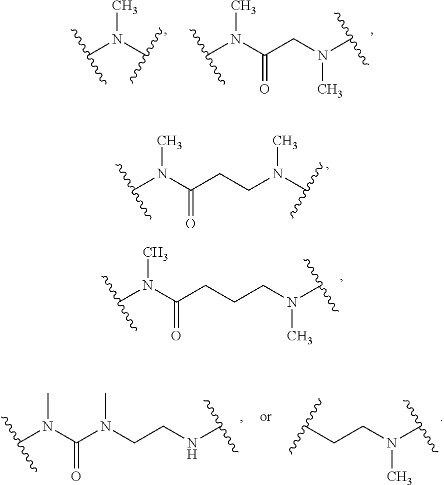

[70] The compound, or a pharmaceutically acceptable salt thereof, of any one of paragraphs [1] to [66], wherein the linker is or comprises a cyclic moiety.
[71] The compound, or a pharmaceutically acceptable salt thereof, of paragraph [70], wherein the linker has the structure of Formula IIb:

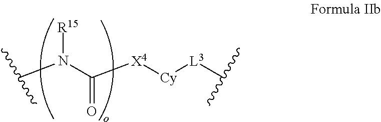

wherein o is 0 or 1;
R¹⁵ is hydrogen, optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.
[72] The compound, or a pharmaceutically acceptable salt thereof, of paragraph [71], wherein the linker has the structure:

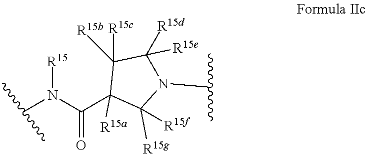

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.
[73] The compound, or a pharmaceutically acceptable salt thereof, of paragraph [72], wherein the linker has the structure:

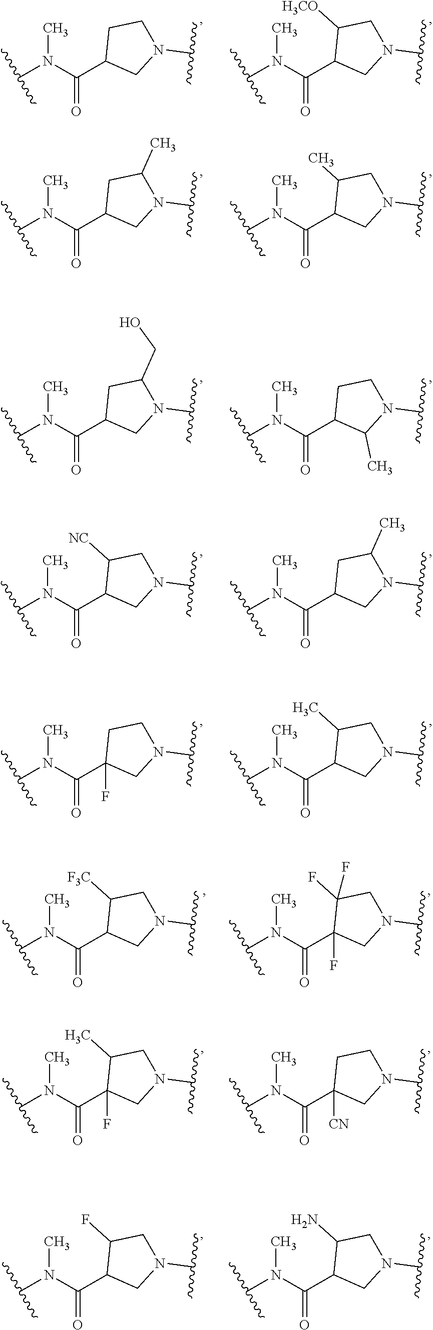

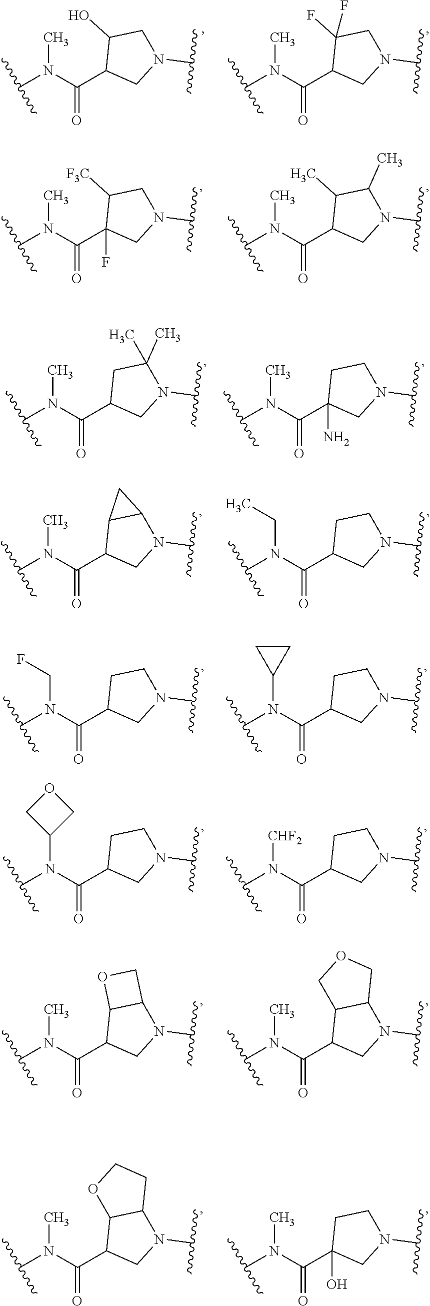

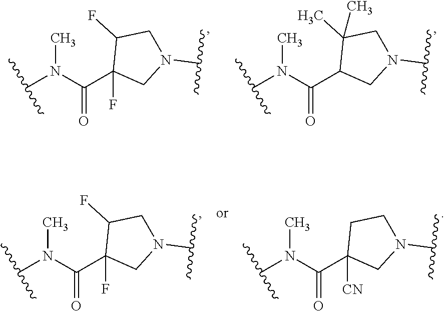 [SMILES: *N1CC(F)C(F)(C(=O)N(*)C)C1 ; *N1CC(C(=O)N(*)C)C(C)(C)C1 ; *N1CC(F)C(F)(C(=O)N(*)C)C1 ; *N1CCC(C#N)(C(=O)N(*)C)C1]

[74] The compound, or a pharmaceutically acceptable salt thereof, of paragraph [71], wherein the linker has the structure:

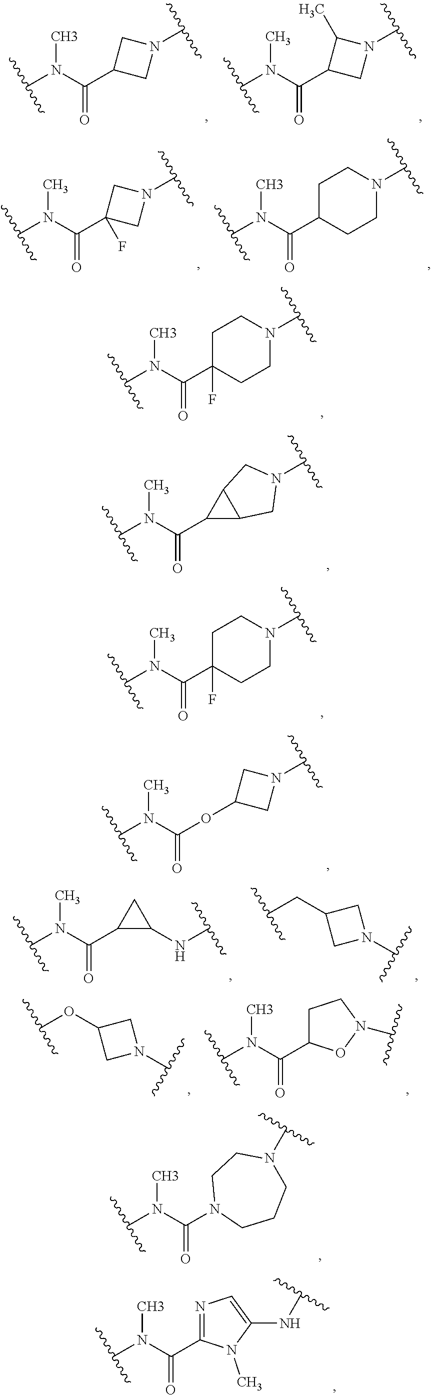

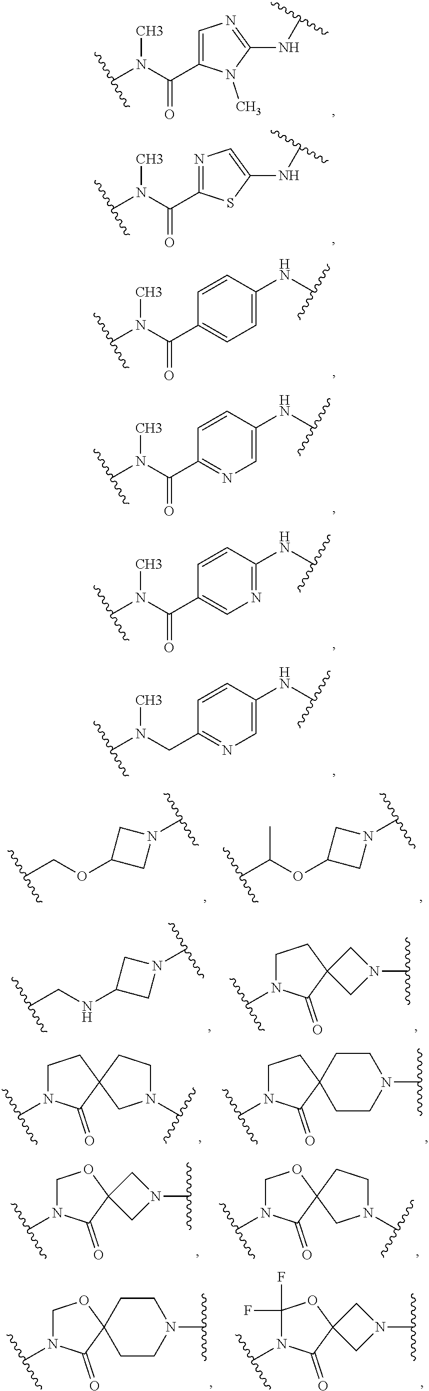

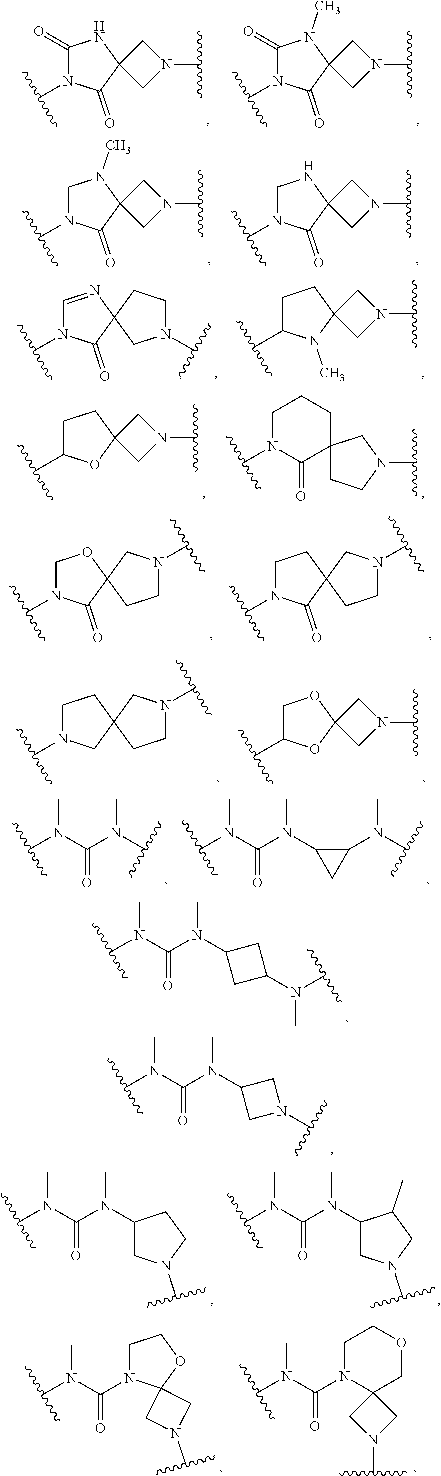

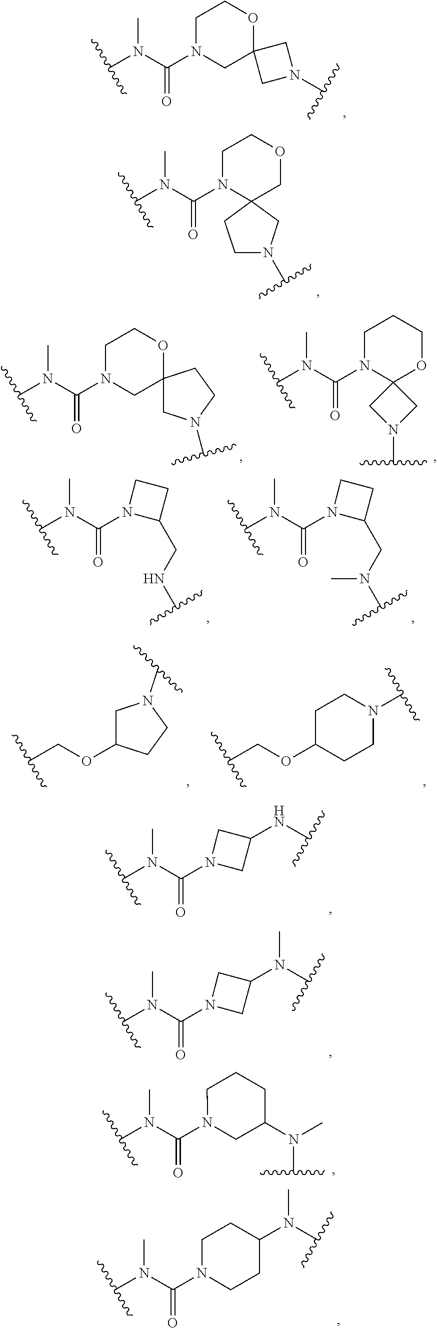

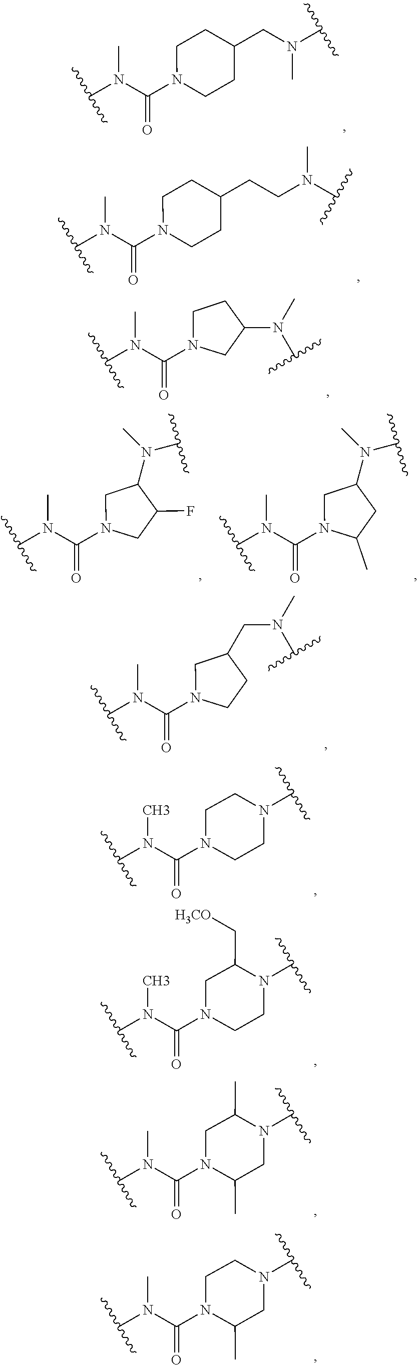

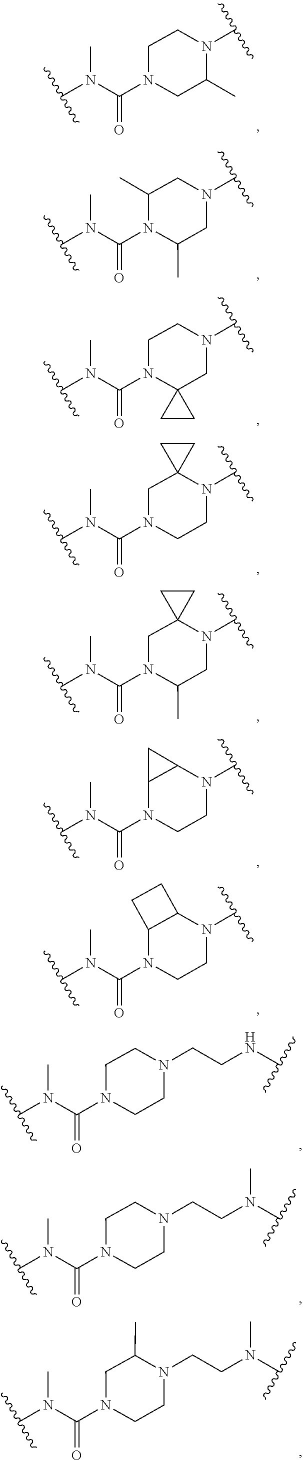

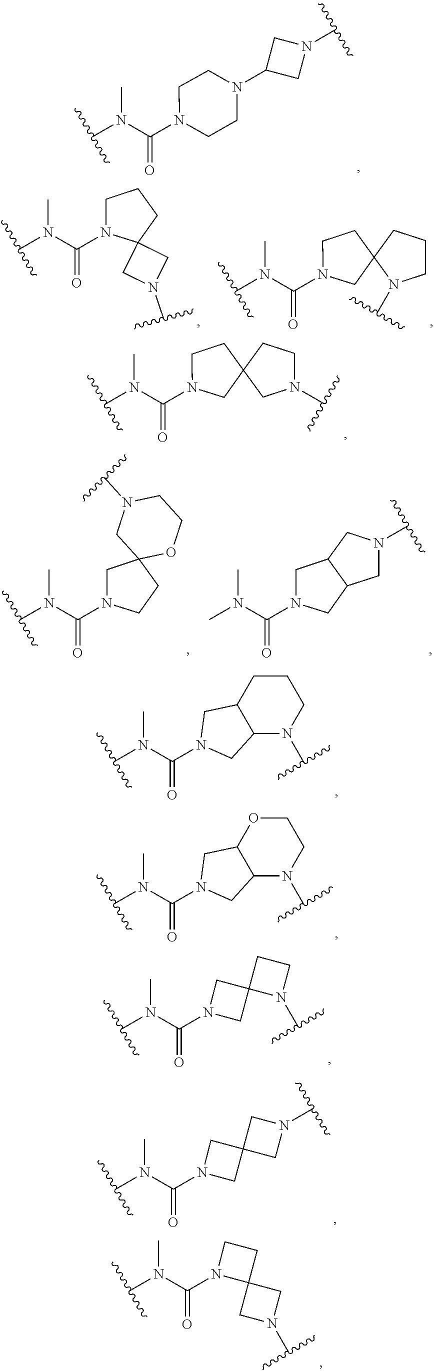

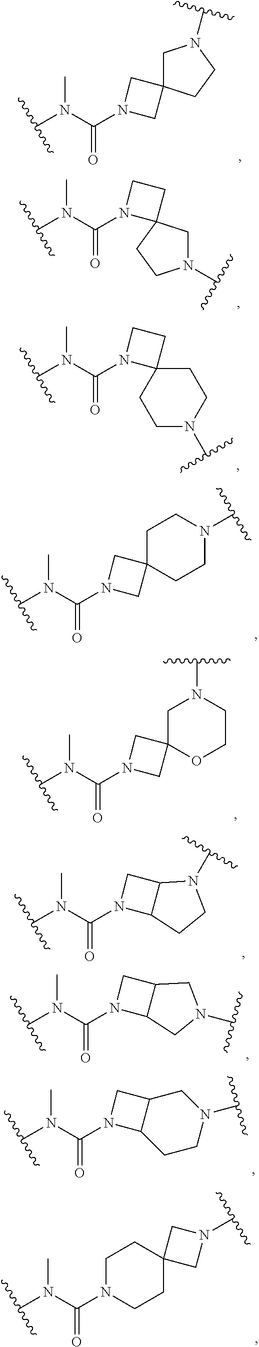

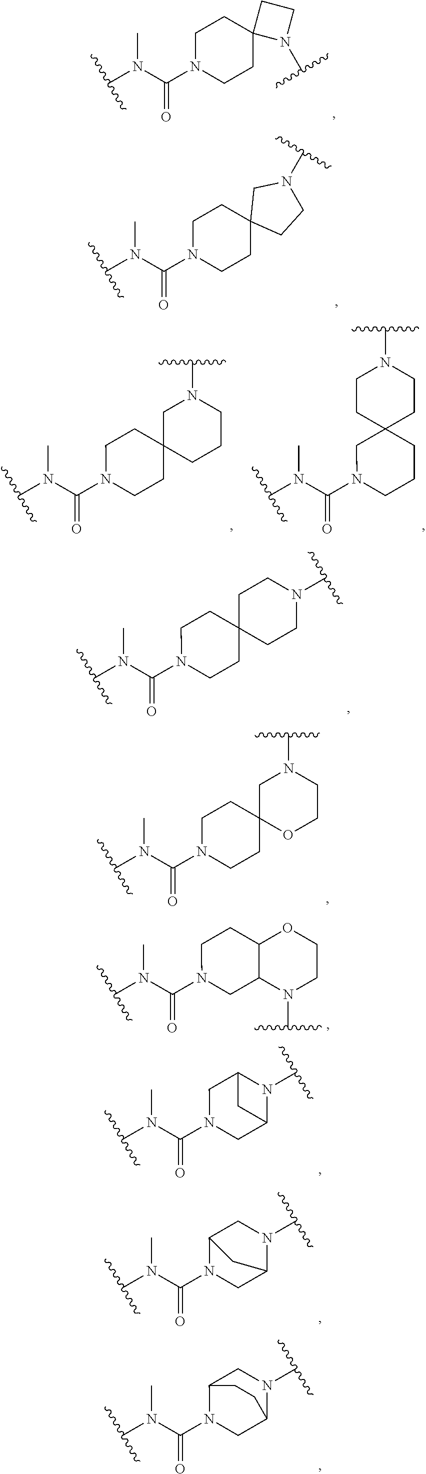

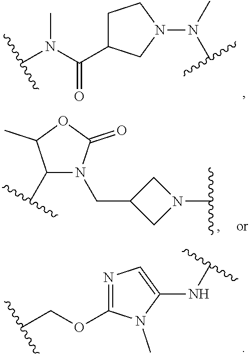

[75] The compound, or a pharmaceutically acceptable salt thereof, of any one of paragraphs [1] to [74], wherein W is a cross-linking group comprising a vinyl ketone.
[76]. The compound, or a pharmaceutically acceptable salt thereof, of paragraph [75], wherein W has the structure of Formula IIIa:

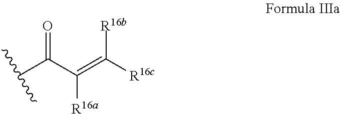

wherein R^{16a}, R^{16b}, and R^{16c} are, independently, hydrogen, —CN, halogen, or —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl.
[77] The compound, or a pharmaceutically acceptable salt thereof, of paragraph [76], wherein W is:

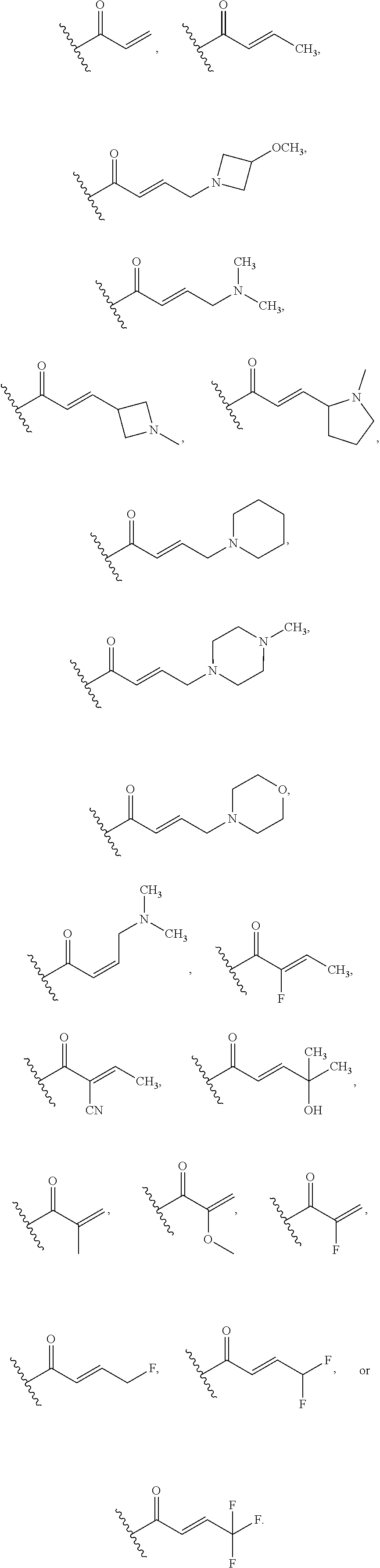

[78] The compound, or a pharmaceutically acceptable salt thereof, of any one of paragraphs [1] to [74], wherein W is a cross-linking group comprising an ynone.
[79] The compound, or a pharmaceutically acceptable salt thereof, of paragraph [78], wherein W has the structure of Formula IIIb:

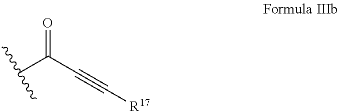

wherein R¹⁷ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated cycloalkyl, or a 4 to 7-membered saturated heterocycloalkyl.
[80] The compound, or a pharmaceutically acceptable salt thereof, of paragraph [79], wherein W is:

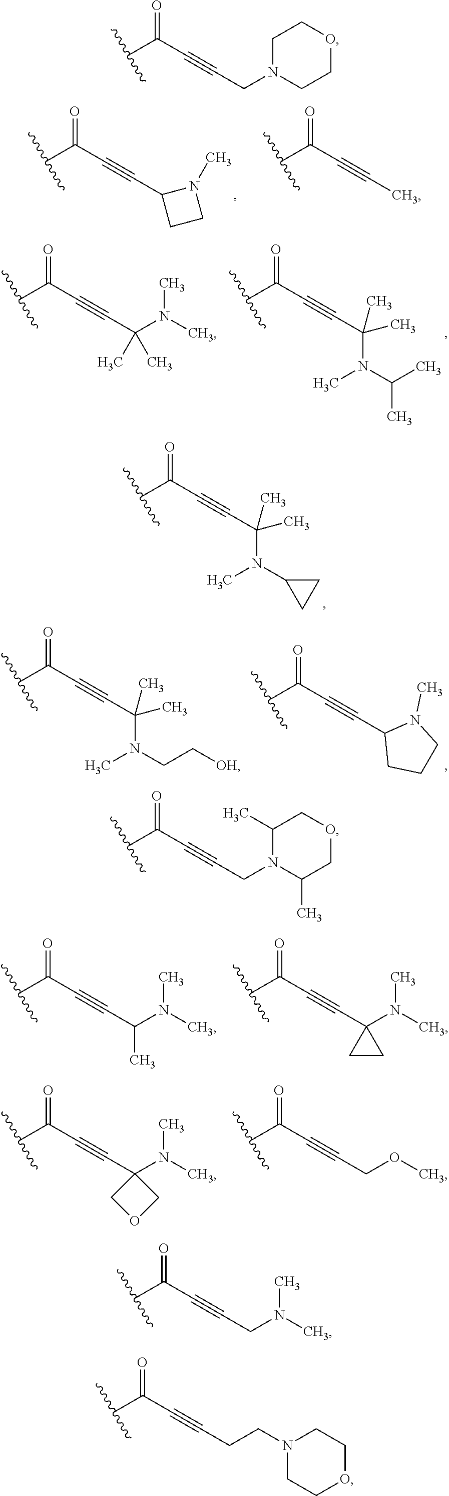

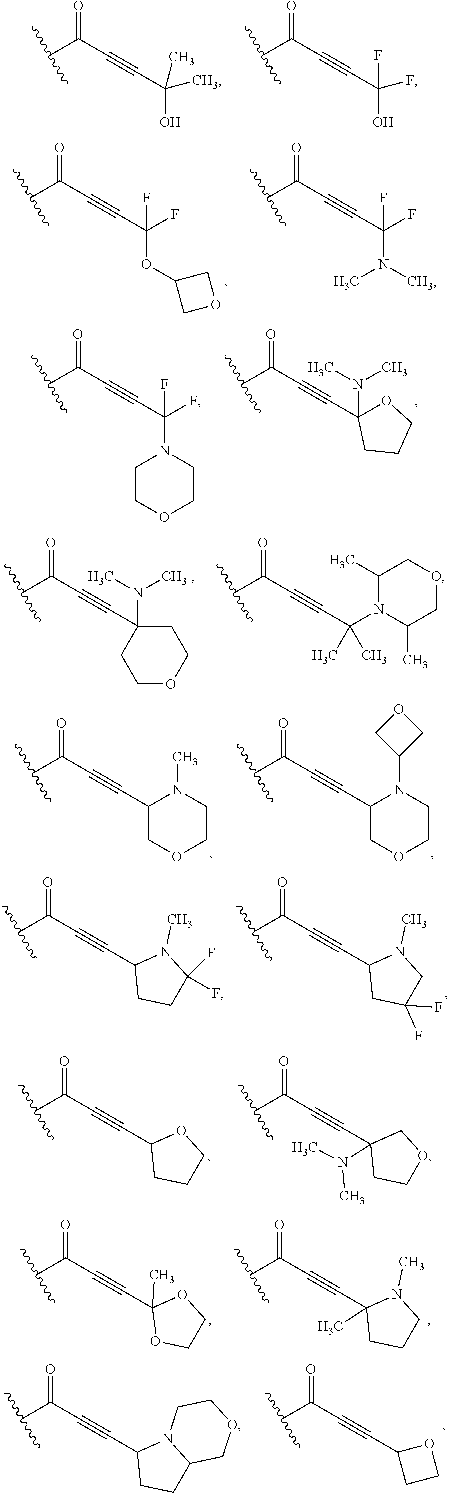

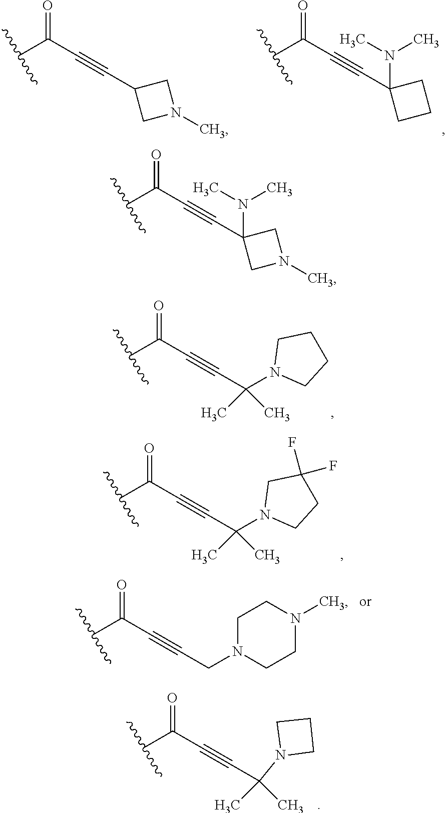

[81] The compound, or a pharmaceutically acceptable salt thereof, of any one of paragraphs [1] to [74], wherein W is a cross-linking group comprising a vinyl sulfone.
[82] The compound, or a pharmaceutically acceptable salt thereof, of paragraph [81], wherein W has the structure of Formula IIIc:

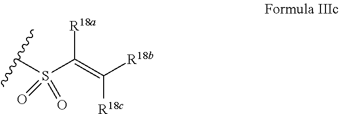

wherein R^{18a}, R^{18b}, and R^{18c} are, independently, hydrogen, —CN, or —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl.
[83] The compound, or a pharmaceutically acceptable salt thereof, of paragraph [82], wherein W is:

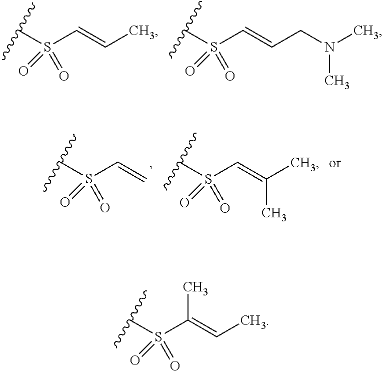

[84] The compound, or a pharmaceutically acceptable salt thereof, of any one of paragraphs [1] to [74], wherein W is a cross-linking group comprising an alkynyl sulfone.
[85] The compound, or a pharmaceutically acceptable salt thereof, of paragraph [84], wherein W has the structure of Formula IIId:

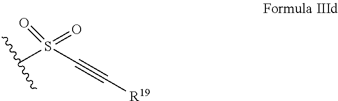

wherein R¹⁹ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl, or a 4 to 7-membered saturated heterocycloalkyl.
[86] The compound, or a pharmaceutically acceptable salt thereof, of paragraph [85], wherein W is:

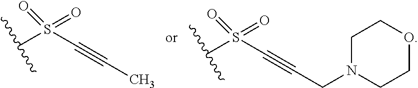

[87] The compound, or a pharmaceutically acceptable salt thereof, of any one of paragraphs [1] to [74], wherein W has the structure of Formula Ille:

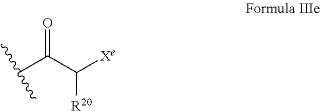

wherein X^{e} is a halogen; and
R²⁰ is hydrogen, —C₁-C₃ alkyl optionally substituted with one or more substituents independently selected from-OH, —O—C₁-C₃ alkyl, —NH₂, —NH(C₁-C₃ alkyl), —N(C₁-C₃ alkyl) 2, or a 4 to 7-membered saturated heterocycloalkyl.
[88] A compound, or a pharmaceutically acceptable salt thereof, selected from Table 1 or Table 2.
[89] A pharmaceutical composition comprising a compound, or a pharmaceutically acceptable salt thereof, of any one of paragraphs [1] to [88], and a pharmaceutically acceptable excipient.
[90] A conjugate, or salt thereof, comprising the structure of Formula IV:

M-L-P Formula IV
wherein L is a linker;
P is a monovalent organic moiety; and
M has the structure of Formula V:

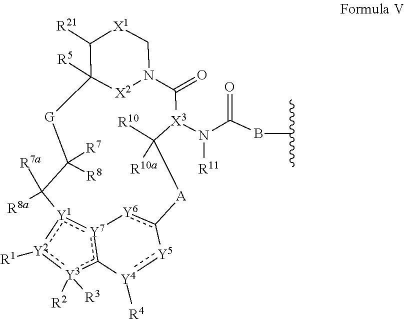

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R⁹′, or >CR⁹R⁹′ where the carbon is bound to the carbonyl carbon of-N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2; R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl; Y¹ is C, CH, or N; Y², Y³, Y⁴, and Y⁷ are, independently, C or N; Y⁵ is CH, CH₂, or N; Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or

R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is H or C₁-C₃ alkyl.
[91] The conjugate of paragraph [90], or salt thereof, wherein M has the structure of Formula Vd:

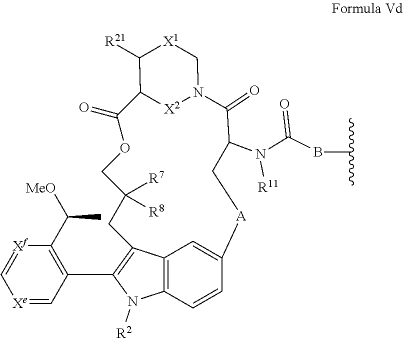

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.
[92] The conjugate of paragraph [91], or salt thereof, wherein M has the structure of Formula Ve:

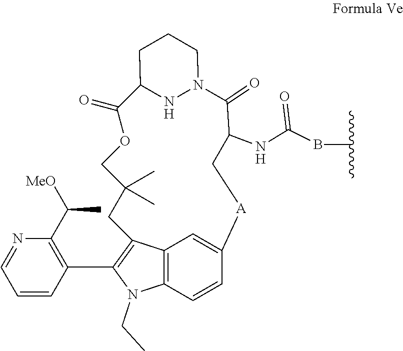

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of-NHC(O)—, optionally substituted 3 to 6-membered cycloalkylene, optionally substituted 3 to 6-membered heterocycloalkylene, optionally substituted 6-membered arylene, or 5 to 6-membered heteroarylene; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[93] The conjugate, or salt thereof, of any one of paragraphs [90] to [92], wherein the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A².
[94] The conjugate, or salt thereof, of any one of paragraphs [90] to [93], wherein the monovalent organic moiety is a protein.
[95] The conjugate, or salt thereof, of paragraph [94], wherein the protein is a Ras protein.
[96] The conjugate, or salt thereof, of paragraph [95], wherein the Ras protein is K-Ras G12C, K-Ras G13C, H-Ras G12C, H-Ras G13C, N-Ras G12C, or N-Ras G13C.
[97] The conjugate, or salt thereof, of any one of paragraphs [93] to [96], wherein the linker is bound to the monovalent organic moiety through a bond to a sulfhydryl group of an amino acid residue of the monovalent organic moiety.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:
CH₂Cl₂, DCM Methylene chloride, Dichloromethane
CH₃CN, MeCN Acetonitrile
CuI Copper (I) iodide
DIPEA Diisopropylethyl amine
DMF N,N-Dimethylformamide
EtOAc Ethyl acetate
h hour
H₂O Water
HCl Hydrochloric acid
K₃PO₄ Potassium phosphate (tribasic)
MeOH Methanol
Na₂SO₄ Sodium sulfate
NMP N-methyl pyrrolidone
Pd(dppf)Cl₂ [1,1′-Bis(diphenylphosphino) ferrocene]dichloropalladium (II)

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

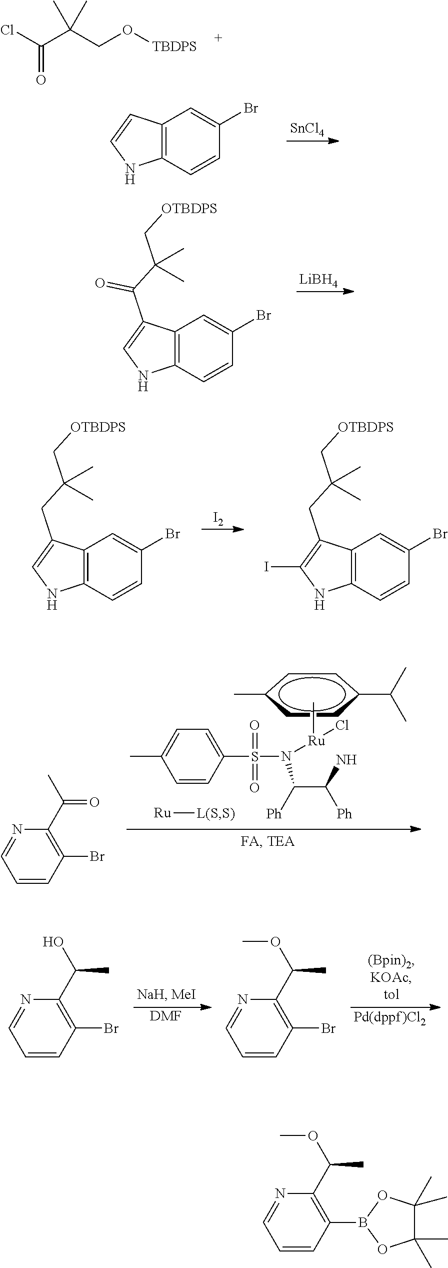

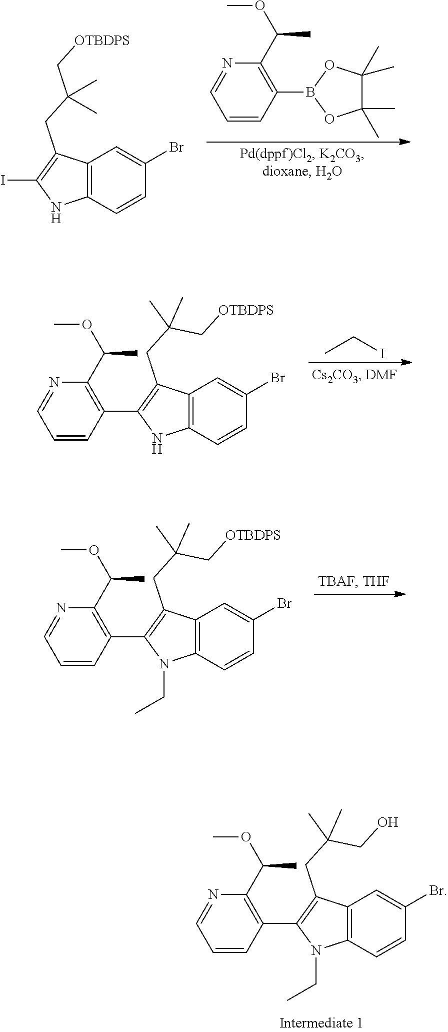

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄, and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na] calc'd for C₂₉H₃₂BrNO₂SiNa 556.1; found 556.3.

Step 2. To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered, and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C₂₉H₃₄BrNOSi 519.2; found 520.1; ¹H NMR (400 MHZ, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄, and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHZ, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄, and filtered. The filtrate was concentrated under reduced pressure and the residue was purified by silica gel column chromatography to give (1S)-1-(3-bromopyridin-2-yl) ethanol (100 g, 74% yield) a an oil. LCMS (ESI): m/z [M+H] calc'd for C₇H₈BrNO 201.1; found 201.9.

Step 5. To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z [M+H] calc'd for C₈H₁₀BrNO 215.0; found 215.9.

Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis (pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl) pyridine (100 g, 63% yield) as a semi-solid. LCMS (ESI): m/z [M+H] calc'd for C₁₄H₂₂BNO₃ 263.2; found 264.1.

Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl) pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol), and H₂O (280 mL) in portions. The mixture was heated to 85° C. and stirred for 4 h, then cooled, H₂O (5 L) added, and the mixture extracted with EtOAc (3×2 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.

Step 8. To a stirred mixture of 5-bromo-3-[3-[(tert-butyldiphenylsilyl)oxy]-2,2-dimethylpropyl]-2-[2-[(1 S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.

Step 9. To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L), and extracted with EtOAc (3×1.5 L). The combined organic layers were washed with brine (2×1 L), dried over anhydrous Na₂SO₄, and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI): m/z [M+H] calc'd for C₂₃H₂₉BrN₂O₂ 444.1; found 445.1.

### Intermediate 1. Alternative Synthesis Through Fisher Indole Route

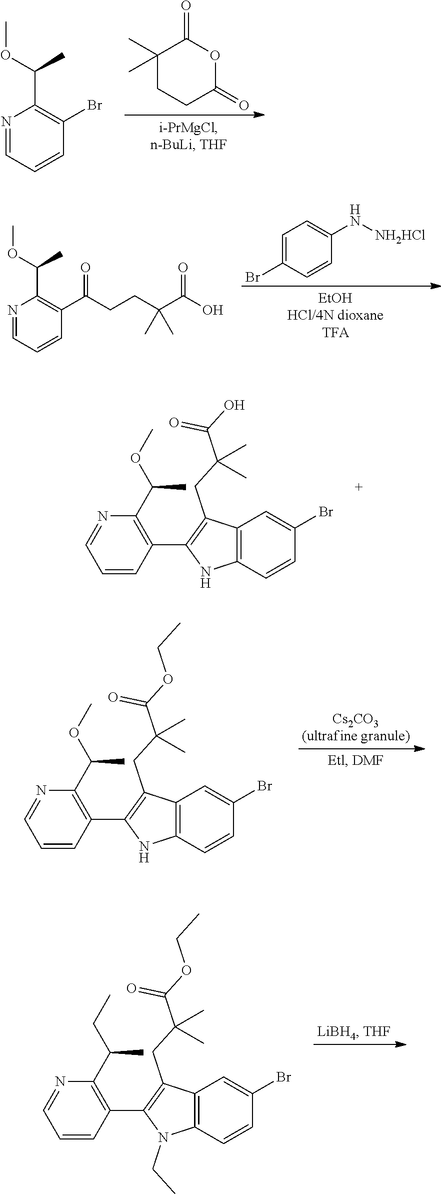

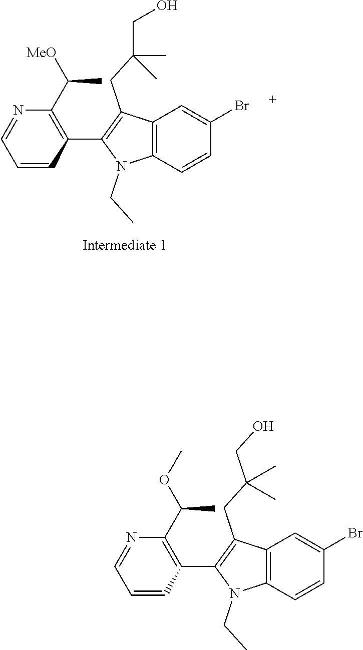

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure, and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.

Step 2. To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl) hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure, and the residue was dissolved in TFA (0.78 L). The mixture was heated to 60° C. and stirred for 1.5 h, concentrated under reduced pressure, and the residue adjusted to pH ˜5 with saturated NaHCO₃, then extracted with EtOAc (3×1.5 L). The combined organic layers were dried over anhydrous Na₂SO₄, filtered, the filtrate concentrated under reduced pressure, and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

Step 3. To a mixture of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄, and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.

Step 4. To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄, and filtered. The filtrate was concentrated under reduced pressure and the residue was purified by silica gel column chromatography to give to two atropisomers (as single atropisomers) of 3-(5-bromo-1-ethyl-2-(2-((S)-1-methoxyethyl) pyridin-3-yl)-1H-indol-3-yl)-2,2-dimethylpropan-1-ol (60 g, 38% yield) and (40 g, 26% yield) both as solids. LCMS (ESI): m/z [M+H] calc'd for C₂₃H₂₉BrN₂O₂ 444.1; found 445.2.

### Intermediate 2 and Intermediate 4. Synthesis of(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate

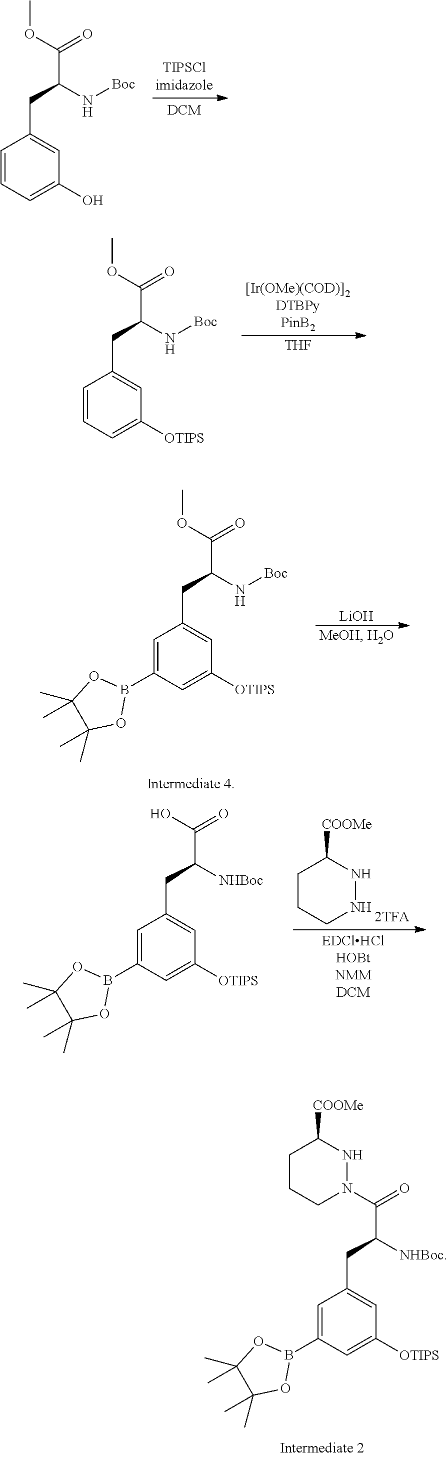

Step 1. To a mixture of(S)-methyl 2-(tert-butoxycarbonylamino)-3-(3-hydroxyphenyl) propanoate (10.0 g, 33.9 mmol) in DCM (100 mL) was added imidazole (4.6 g, 67.8 mmol) and TIPSCl (7.8 g, 40.7 mmol). The mixture was stirred at rt overnight then diluted with DCM (200 mL) and washed with H₂O (150 mL×3). The organic layer was dried over anhydrous Na₂SO₄, filtered, concentrated under reduced pressure, and the residue was purified by silica gel column chromatography to give (S)-methyl 2-(tert-butoxycarbonylamino)-3-(3-(triisopropylsilyloxy)phenyl)-propanoate (15 g, 98% yield) as an oil. LCMS (ESI): m/z [M+Na] calc'd for C₂₄H₄₁NO₅SiNa 474.3; found 474.2.

Step 2. A mixture of(S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol), and 4-tert-butyl-2-(4-tert-butyl-2-pyridyl) pyridine (1.3 g, 5.0 mmol) was purged with Ar (×3), then THF (75 mL) was added and the mixture placed under an atmosphere of Ar and sealed. The mixture was heated to 80° C. and stirred for 16 h, concentrated under reduced pressure, and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI): m/z [M+Na] calc'd for C₃₀H₅₂BNO₇SiNa 600.4; found 600.4; ¹H NMR (300 MHZ, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

Step 3. To a mixture of triisopropylsilyl(S)-2-((tert-butoxycarbonyl) amino)-3-(3-(4,4,5,5-tetramethyl-1,3,2-dioxaborolan-2-yl)-5-((triisopropylsilyl)oxy) phenyl) propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to pH ˜5 with 1M HCl and extracted with EtOAc (250 mL×2). The combined organic layers were washed with brine (100 mL×3), dried over anhydrous Na₂SO₄, filtered, and the filtrate concentrated under reduced pressure to give (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (3.7 g, 95% yield), which was used directly in the next step without further purification. LCMS (ESI): m/z [M+NH₄] calc'd for C₂₉H₅₀BNO₇SINH₄ 581.4; found 581.4.

Step 4. To a mixture of methyl(S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (24 g, 42.6 mmol) in DCM (50 mL) then HOBt (1.21 g, 9.0 mmol) and EDCl HCl salt (12.9 g, 67.6 mmol). The mixture was warmed to rt and stirred for 16 h, then diluted with DCM (200 mL) and washed with H₂O (3×150 mL). The organic layer was dried over anhydrous Na₂SO, filtered, the filtrate concentrated under reduced pressure, and the residue was purified by silica gel column chromatography to give methyl(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate (22 g, 71% yield) as an oil. LCMS (ESI): m/z [M+H] calc'd for C₃₅H₆₀BN₃O₈Si 689.4; found 690.5.

Intermediate 3. Synthesis of N—((S)-1-acryloylpyrrolidine-3-carbonyl)-N-methyl-L-valine

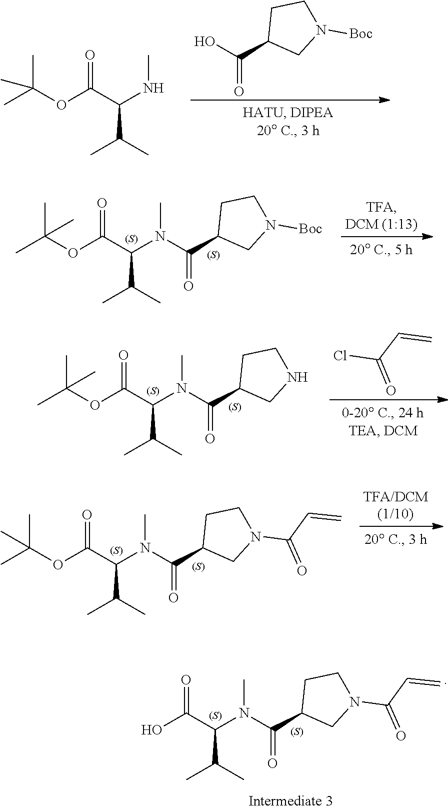

Step 1. To a mixture of(S)-1-(tert-butoxycarbonyl) pyrrolidine-3-carboxylic acid (2.2 g, 10.2 mmol) in DMF (10 mL) at rt was added HATU (7.8 g, 20.4 mmol) and DIPEA (5 mL). After stirring at rt for 10 min, tert-butyl methyl-L-valinate (3.8 g, 20.4 mmol) in DMF (10 mL) was added. The mixture was stirred at rt for 3 h, then diluted with DCM (40 mL) and H₂O (30 mL). The aqueous and organic layers were separated and the organic layer was washed with H₂O (3×30 mL), brine (30 mL), dried over anhydrous Na₂SO₄, and filtered. The filtrate was concentrated under reduced pressure and the residue was purified by silica gel column chromatography to give (S)-tert-butyl 3-(((S)-1-(tert-butoxy)-3-methyl-1-oxobutan-2-yl) (methyl) carbamoyl) pyrrolidine-1-carboxylate (3.2 g, 82% yield) as an oil. LCMS (ESI): m/z [M+Na] calc'd for C₂₀H₃₆N₂O₅Na 407.3; found 407.2.

Step 2. A mixture of(S)-tert-butyl 3-(((S)-1-(tert-butoxy)-3-methyl-1-oxobutan-2-yl) (methyl) carbamoyl) pyrrolidine-1-carboxylate (3.2 g, 8.4 mmol) in DCM (13 mL) and TFA (1.05 g, 9.2 mmol) was stirred at rt for 5 h. The mixture was concentrated under reduced pressure to give (S)-tert-butyl 3-methyl-2-((S)—N-methylpyrrolidine-3-carboxamido) butanoate (2.0 g, 84% yield) as an oil. LCMS (ESI): m/z [M+H] calc'd for C₁₅H₂₈N₂O₃ 284.2; found 285.2.

Step 3. To a mixture of(S)-tert-butyl 3-methyl-2-((S)—N-methylpyrrolidine-3-carboxamido) butanoate (600 mg, 2.1 mmol) in DCM (6 mL) at 0° C. was added TEA (342 mg, 3.36 mmol). After stirring at 0° C. for 10 mins, acryloyl chloride (284 mg, 3.2 mmol) in DCM (10 mL) was added. The mixture was warmed to rt and stirred for 24 h, then diluted with DCM (30 mL) and H₂O (30 mL). The aqueous and organic layers were separated and the organic layer was washed with H₂O (3×30 mL), brine (30 mL), dried over anhydrous Na₂SO₄, and filtered. The filtrate was concentrated under reduced pressure and the residue was purified by silica gel column chromatography to give tert-butyl N—((S)-1-acryloylpyrrolidine-3-carbonyl)-N-methyl-L-valinate (500 mg, 70% yield) as an oil.

Step 4. To a mixture of tert-butyl N—((S)-1-acryloylpyrrolidine-3-carbonyl)-N-methyl-L-valinate (100 mg, 0.29 mmol) in DCM (3.0 mL) at 15° C. was added TFA (0.3 mL). The mixture was warmed to rt and stirred for 5 h, then the mixture was concentrated under reduced pressure to give N—((S)-1-acryloylpyrrolidine-3-carbonyl)-N-methyl-L-valine (150 mg) as a solid. The crude product was used directly in the next step without further purification. LCMS (ESI): m/z [M+H] calc'd for C₁₄H₂₂N₂O₄ 282.2; found 283.2.

### Intermediate 5. Synthesis of tert-butyl ((63S,4S)-11-ethyl-12-(2-((S)-1-methoxyethyl) pyridin-3-yl)-10,10-dimethyl-5,7-dioxo-2⁵-((triisopropylsilyl)oxy)-61,62,63,64,65,66-hexahydro-11H-8-oxa-1 (5,3)-indola-6 (1,3)-pyridazina-2 (1,3)-benzenacycloundecaphane-4-yl) carbamate

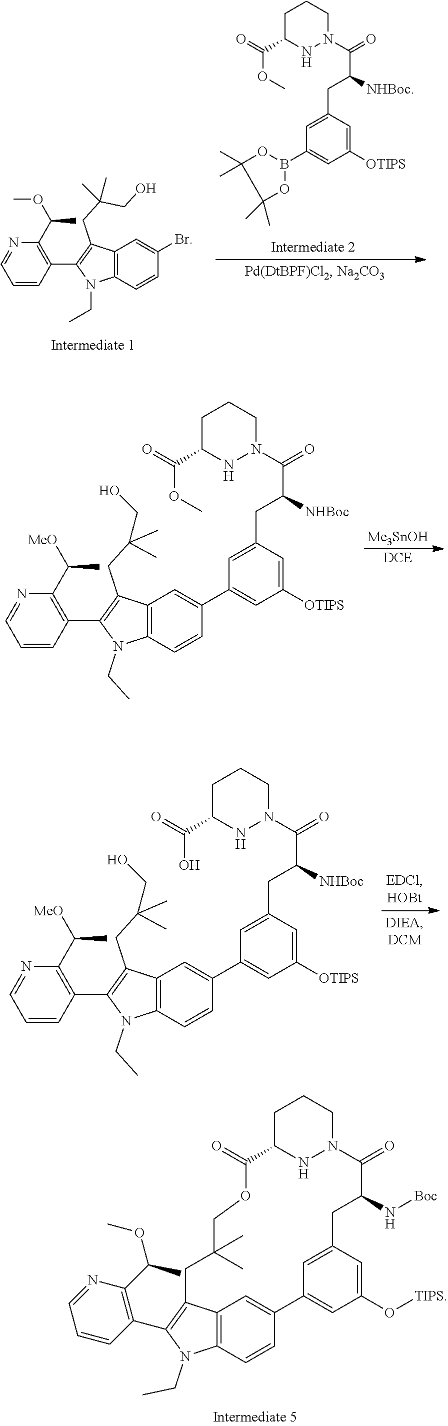

Step 1. To a stirred mixture of 3-(5-bromo-1-ethyl-2-[2-[(1S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at rt under an atmosphere of Ar was added Na₂CO₃ (17.9 g, 168.4 mmol), Pd(DtBPF)Cl₂ (4.39 g, 6.7 mmol), and H₂O (150.00 mL) in portions. The mixture was heated to 85° C. and stirred for 3 h, cooled, diluted with H₂O (2 L), and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI): m/z [M+H] calc'd for C₅₂H₇₇N₅O₈Si 927.6; found 928.8.

Step 2. To a stirred mixture of methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at rt was added trimethyltin hydroxide (48.7 g, 269 mmol) in portion. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI): m/z [M+H] calc'd for C₅₁H₇₅N₅O₈Si 913.5; found 914.6.

Step 3. To a stirred mixture of (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (297 g, 2.3 mol), HOBT (51.7 g, 383 mmol) and EDCl (411 g, 2.1 mol) in portions. The mixture was warmed to rt and stirred for 16 h, then diluted with DCM (1 L), washed with brine (3×1 L), dried over anhydrous Na₂SO₄, and filtered. The filtrate was concentrated under reduced pressure and the residue was purified by silica gel column chromatography to give tert-butyl ((63S,4S)-11-ethyl-12-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (36 g, 42% yield) as a solid. LCMS (ESI): m/z [M+H] calc'd for C₅₁H₇₃N₅O₇Si 895.5; found 896.5.

### Intermediate 6. Synthesis of tert-butyl N-[(8S,14S)-21-iodo-18,18-dimethyl-9,15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]nonacosa-1 (26),2,4,6 (29),20,23 (27),24-heptaen-8-yl]carbamate

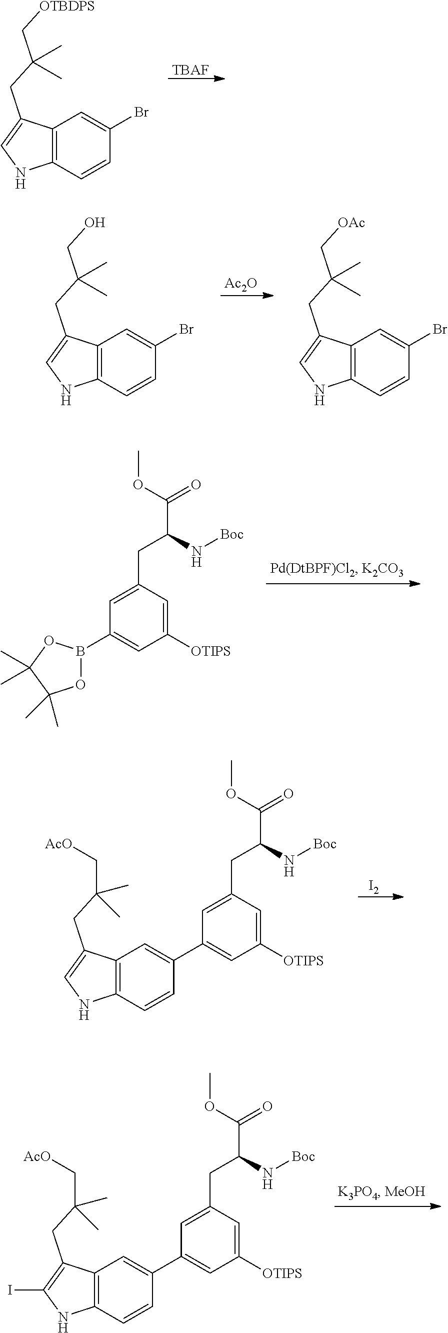

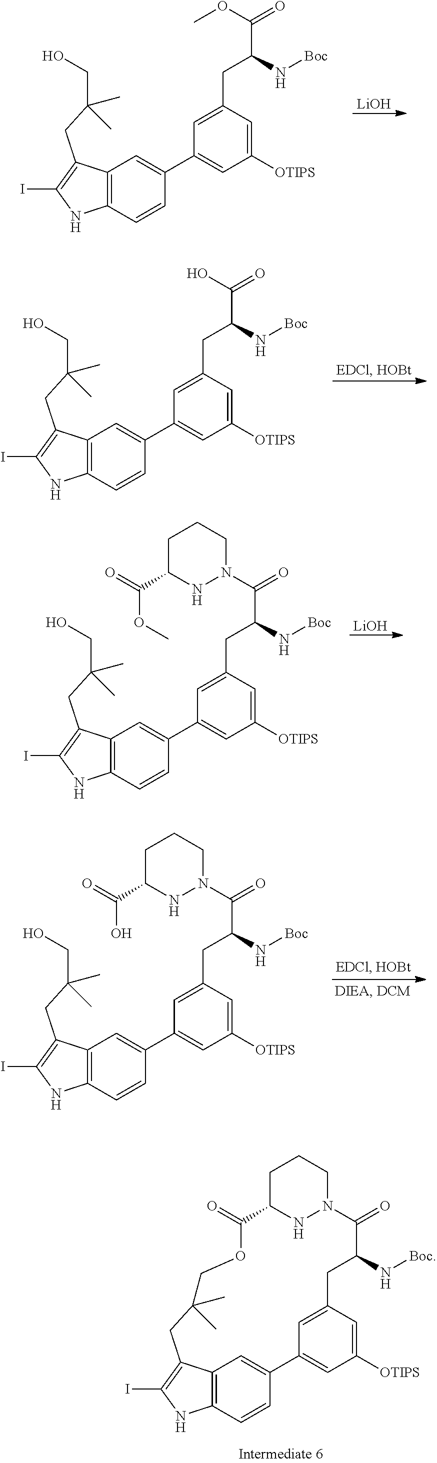

Step 1. This reaction was undertaken on 5-batches in parallel on the scale illustrated below.

Into a 2 L round-bottom flasks each were added 5-bromo-3-[3-[(tert-butyldiphenylsilyl)oxy]-2,2-dimethylpropyl]-1H-indole (100 g, 192 mmol) and TBAF (301.4 g, 1.15 mol) in THF (1.15 L) at rt. The resulting mixture was heated to 50° C. and stirred for 16 h, then the mixture was concentrated under reduced pressure. The combined residues were diluted with H₂O (5 L) and extracted with EtOAc (3×2 L). The combined organic layers were washed with brine (2×1.5 L), dried over anhydrous Na₂SO₄, and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI): m/z [M+H] calc'd for C₁₃H₁₆BrNO 281.0 and 283.0; found 282.1 and 284.1.

Step 2. This reaction was undertaken on two batches in parallel on the scale illustrated below.

To a stirred mixture of 3-(5-bromo-1H-indol-3-yl)-2,2-dimethylpropan-1-ol (135 g, 478 mmol) and TEA (145.2 g, 1.44 mol) in DCM (1.3 L) at 0° C. under an atmosphere of N₂ was added Ac₂O (73.3 g, 718 mmol) and DMAP (4.68 g, 38.3 mmol) in portions. The resulting mixture was stirred for 10 min at 0° C., then washed with H₂O (3×2 L). The organic layers from each experiment were combined and washed with brine (2×1 L), dried over anhydrous Na₂SO₄, and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. ¹H NMR (400 MHZ, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

Step 3. This reaction was undertaken on four batches in parallel on the scale illustrated below.

Into a 2 L round-bottom flasks were added methyl (2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol), and Pd(DtBPF)Cl₂ (7.05 g, 10.8 mmol) at rt under an atmosphere of Ar. The resulting mixture was heated to 65° C. and stirred for 2 h, then diluted with H₂O (10 L) and extracted with EtOAc (3×3 L). The combined organic layers were washed with brine (2×2 L), dried over anhydrous Na₂SO₄, and filtered. The filtrate was concentrated under reduced pressure and the residue was purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (500 g, 74% yield) as an oil. LCMS (ESI): m/z [M+Na] calc'd for C₃₉H₅₈N₂O₇SiNa 717.4; found 717.3.

Step 4. This reaction was undertaken on three batches in parallel on the scale illustrated below.

To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (150 g, 216 mmol) and NaHCO₃ (21.76 g, 259 mmol) in THF (1.5 L) was added AgOTf (66.5 g, 259 mmol) in THE dropwise at 0° C. under an atmosphere of nitrogen. I₂ (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄, and filtered. The filtrate was concentrated under reduced pressure and the residue was purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (420 g, 71% yield) as an oil. LCMS (ESI): m/z [M+Na] calc'd for C₃₉H₅₇IN₂O₇SiNa, 843.3; found 842.9.

Step 5. This reaction was undertaken on three batches in parallel on the scale illustrated below.

To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (140 g, 171 mmol), MeOH (1.4 L) and K₃PO₄ (108.6 g, 512 mmol) at 0° C. The mixture was warmed to rt and stirred for 1 h, then the combined experiments were diluted with H₂O (9 L) and extracted with EtOAc (3×3 L). The combined organic layers were washed with brine (2×2 L), dried over anhydrous Na₂SO₄, filtered, and the filtrate was concentrated under reduced pressure to give methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI): m/z [M+Na] calc'd for C₃₇H₅₅IN₂O₆SiNa 801.3; found 801.6.

Step 6. This reaction was undertaken on three batches in parallel on the scale illustrated below.

To a stirred mixture of methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to rt and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered, and the filtrate was concentrated under reduced pressure to give (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI): m/z [M+Na] calc'd for C₃₆H₅₃IN₂O₆SiNa 787.3; found 787.6.

Step 7. To a stirred mixture of (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCl (170 g, 889 mmol), and HOBT (12.0 g, 88.9 mmol) portionwise at 0° C. The mixture was warmed to rt and stirred for 16 h, then washed with H₂O (3×2.5 L), brine (2×1 L), dried over anhydrous Na₂SO₄ and filtered. The filtrate was concentrated under reduced pressure and the residue was purified by column chromatography to give methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI): m/z [M+H] calc'd for C₄₂H₆₃IN₄O₇Si 890.4; found 890.8.

Step 8. This reaction was undertaken on three batches in parallel on the scale illustrated below.

To a stirred mixture of methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) each added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl and the combined experiments extracted with EtOAc (3×2 L). The combined organic layers were washed with brine (2×1.5 L), dried over anhydrous Na₂SO₄, filtered, and the filtrate was concentrated under reduced pressure to give (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI): m/z [M+H] calc'd for C₄₁H₆₁IN₄O₇Si 876.3; found 877.6.

### Step 9.

This reaction was undertaken on two batches in parallel on the scale illustrated below.

To a stirred mixture of (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (265 g, 2.05 mol), EDCl (394 g, 2.05 mol), and HOBT (37 g, 274 mmol) in portions at 0° C. under an atmosphere of N₂. The mixture was warmed to rt and stirred overnight, then the combined experiments were washed with H₂O (3×6 L), brine (2×6 L), dried over anhydrous Na₂SO₄, and filtered. After filtration, the filtrate was concentrated under reduced pressure. The filtrate was concentrated under reduced pressure and the residue was purified by column chromatography to give tert-butyl N-[(8S,14S)-21-iodo-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (140 g, 50% yield) as a solid. LCMS (ESI): m/z [M+H] calc'd for C₄₁H₅₉IN₄O₆Si 858.9; found 858.3.

### Intermediate 7. Synthesis of(S)-methyl 2-((tert-butoxycarbonyl) amino)-3-(4-(4,4,5,5-tetramethyl-1,3,2-dioxaborolan-2-yl) pyridin-2-yl) propanoate

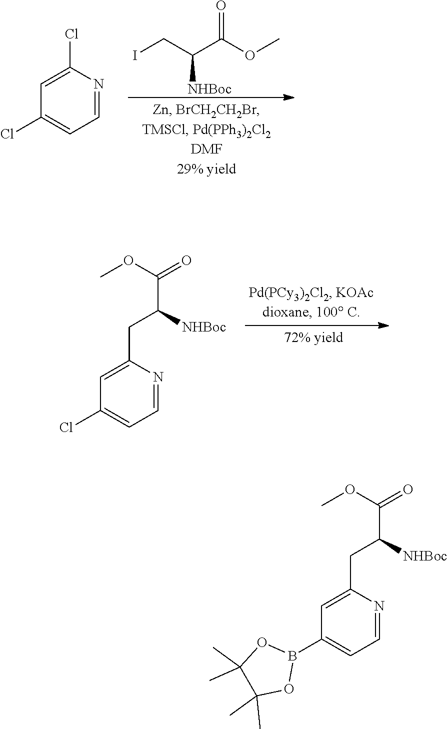

Step 1. Zn dust (28 g, 428 mmol) was added to a 1L, three necked, round bottomed flask, purged with N₂, and heated with a heat gun for 10 min under vacuum. The mixture was cooled to rt, and a solution of 1,2-dibromoethane (1.85 mL, 21.5 mmol) in DMF (90 mL) was added dropwise over 10 min. The mixture was heated at 90° C. for 30 min and re-cooled to rt. TMSCl (0.55 mL, 4.3 mmol) was added, and the mixture was stirred for 30 min at rt, then a mixture of (R)-methyl 2-((tert-butoxycarbonyl) amino)-3-iodopropanoate (22.5 g, 71.4 mmol) in DMF (200 mL) was added dropwise over a period of 10 min. The mixture was heated at 35° C. and stirred for 2 h, then cooled to rt, and 2,4-dichloropyridine (16 g, 109 mmol) and Pd(PPh₃)₂Cl₂ (4 g, 5.7 mmol) added. The mixture was heated at 45° C. and stirred for 2 h, cooled, and filtered, then H₂O (1 L) and EtOAc (0.5 L) were added to the filtrate. The organic and aqueous layers were separated, and the aqueous layer was extracted with EtOAc (2×500 mL). The organic layers were dried over anhydrous Na₂SO₄, filtered, and concentrated under reduced pressure. The crude residue was purified by silica gel column chromatography to give (S)-methyl 2-((tert-butoxycarbonyl) amino)-3-(4-chloropyridin-2-yl) propanoate (6.5 g, 29% yield) as a solid. LCMS (ESI): m/z [M+H] calc'd for C₁₄H₁₉ClN₂O₄ 314.1; found 315.1.

Step 2. To a mixture of(S)-methyl 2-((tert-butoxycarbonyl) amino)-3-(4-chloropyridin-2-yl) propanoate (6.5 g, 20.6 mmol) in 1,4-dioxane (80 mL) at rt under an atmosphere of N₂ was added bis (pinacolato)diboron (6.3 g, 24.7 mmol), KOAc (8.1 g, 82.4 mmol), and Pd(PCy₃)₂Cl₂ (1.9 g, 2.5 mmol). The mixture was heated to 100° C. and stirred for 3 h, then H₂O (100 mL) added and the mixture extracted with EtOAc (3×200 mL). The organic layers were combined, washed with brine (2×100 mL), dried over anhydrous Na₂SO₄, and filtered. The filtrate was concentrated under reduced pressure and the residue was purified by silica gel column chromatography to give (S)-methyl 2-((tert-butoxycarbonyl) amino)-3-(4-(4,4,5,5-tetramethyl-1,3,2-dioxaborolan-2-yl) pyridin-2-yl) propanoate (6 g, 72% yield) as a solid. LCMS (ESI): m/z [M+H] calc'd for C₂₀H₃₁BN₂O₆ 406.2; found 407.3.

### Synthesis of Intermediate 8

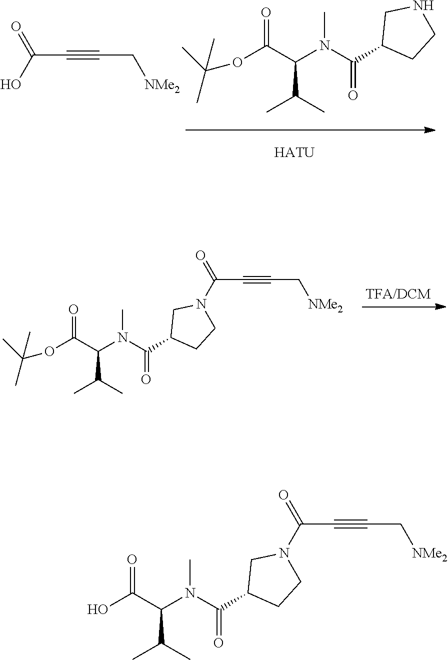

Step 1. To a mixture of 4-(dimethylamino) but-2-ynoic acid (900 mg, 7.0 mmol) in DMF (20 mL) at −5° C. was added tert-butyl N-methyl-N—((S)-pyrrolidine-3-carbonyl)-L-valinate (1.0 g, 3.5 mmol), DIPEA (2.2 g, 17.6 mmol) and HATU (2.7 g, 7.0 mmol) in portions. The mixture was stirred between-5 to 5° C. for 1 h, then diluted with EtOAc (100 mL) and ice-H₂O (100 mL). The aqueous and organic layers were separated and the organic layer was washed with H₂O (3×100 mL), brine (100 mL), dried over anhydrous Na₂SO₄, and filtered. The filtrate was concentrated under reduced pressure and the residue was purified by silica gel column chromatography to give tert-butyl N—((S)-1-(4-(dimethylamino) but-2-ynoyl) pyrrolidine-3-carbonyl)-N-methyl-L-valinate (900 mg, 55% yield) as an oil. LCMS (ESI): m/z [M+H] calc'd for C₂₁H₃₅N₃O₄ 393.5; found 394.3.

Step 2. To a mixture of tert-butyl N—((S)-1-(4-(dimethylamino) but-2-ynoyl) pyrrolidine-3-carbonyl)-N-methyl-L-valinate (260 mg, 0.66 mmol) in DCM (6 mL) was added TFA (3 mL) at rt. The mixture was stirred at rt for 2 h, then the solvent was concentrated under reduced pressure to give (2S)-2-{1-[(3S)-1-[4-(dimethylamino) but-2-ynoyl]pyrrolidin-3-yl]-N-methylformamido}-3-methylbutanoic acid (280 mg) as an impure oil. The crude product was used directly in the next step without further purification. LCMS (ESI): m/z [M+H] calc'd for C₁₇H₂₇N₃O₄ 337.2; found 338.3.

### Synthesis of Intermediate 9

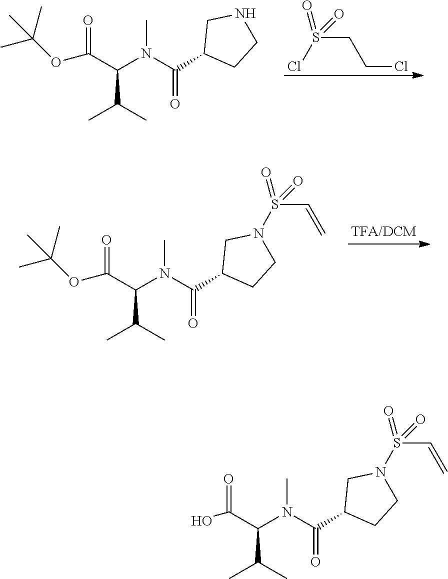 [SMILES: CC(C)[C@@H](C(=O)OC(C)(C)C)N(C)C(=O)[C@H]1CCNC1 ; O=S(=O)(Cl)CCCl ; C=CS(=O)(=O)N1CC[C@H](C(=O)N(C)[C@H](C(=O)OC(C)(C)C)C(C)C)C1 ; C=CS(=O)(=O)N1CC[C@H](C(=O)N(C)[C@H](C(=O)O)C(C)C)C1]

Step 1. To a mixture of tert-butyl N-methyl-N—((S)-pyrrolidine-3-carbonyl)-L-valinate (500 mg, 1.8 mmol) in DCM (8 mL) at 5° C. was added TEA (533 mg, 5.3 mmol) followed by dropwise addition of 2-chloroethane-1-sulfonyl chloride (574 mg, 3.5 mmol) in DCM (2 mL) The mixture was stirred at 5° C. for 1 h, then diluted with H₂O (20 mL) and extracted with EtOAC (3×10 mL). The combined organic layers were washed with brine (10 mL), dried over anhydrous Na₂SO₄, and filtered. The filtrate was concentrated under reduced pressure and the residue was purified by silica gel column chromatography to give tert-butyl N-methyl-N—((S)-1-(vinylsulfonyl) pyrrolidine-3-carbonyl)-L-valinate (300 mg, 45% yield) as an oil. LCMS (ESI): m/z [M+H] calc'd for C₁₇H₃ON₂O₅S 374.2; found 375.2.

Step 2. To a mixture of tert-butyl N-methyl-N—((S)-1-(vinylsulfonyl) pyrrolidine-3-carbonyl)-L-valinate (123 mg, 0.33 mmol) in DCM (3 mL) at rt was added TFA (1 mL). The mixture was stirred at rt for 1 h, then concentrated under reduced pressure to give N-methyl-N—((S)-1-(vinylsulfonyl) pyrrolidine-3-carbonyl)-L-valine (130 mg, crude) as a solid, which was used directly in the next step without further purification. LCMS (ESI): m/z [M+H] calc'd for C₁₃H₂₂N₂O₅S 318.1; found 319.1.

### Synthesis of Intermediate 10

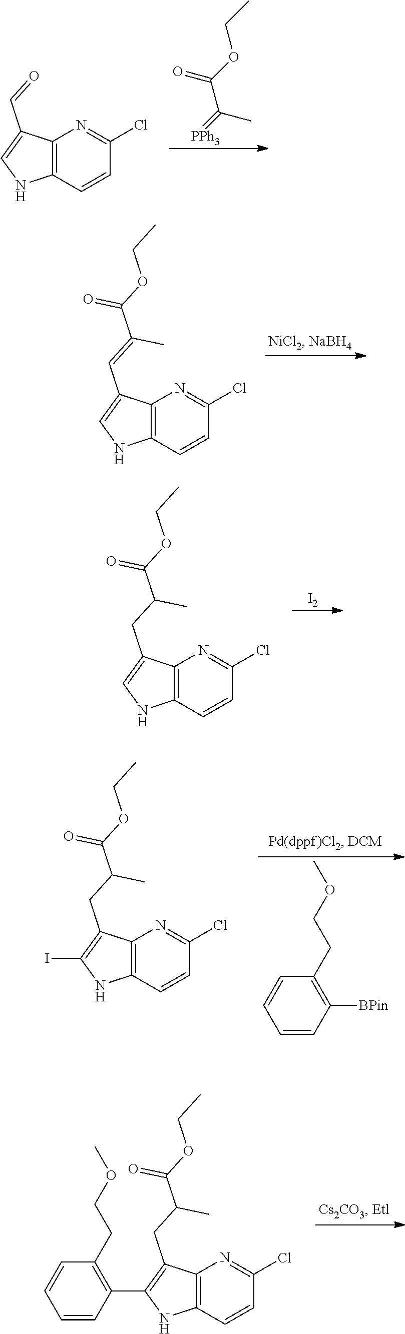

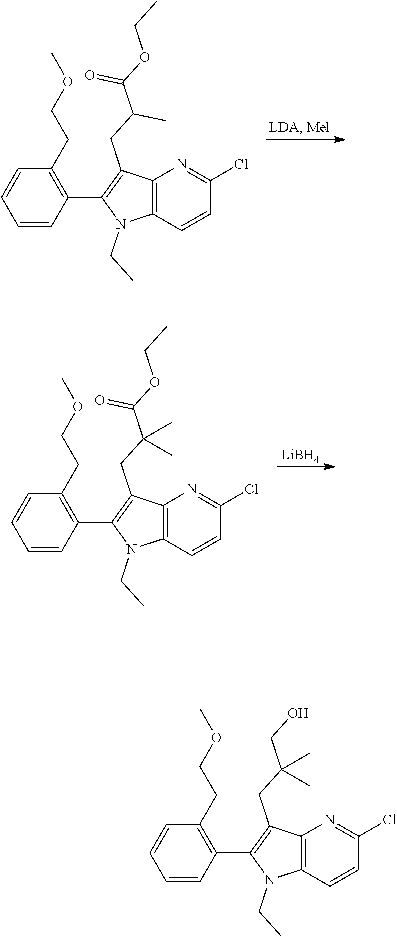 [SMILES: CCOC(=O)C(C)Cc1c(-c2ccccc2CCOC)n(CC)c2ccc(Cl)nc12 ; CCOC(=O)C(C)(C)Cc1c(-c2ccccc2CCOC)n(CC)c2ccc(Cl)nc12 ; "LiBH4" ; CCn1c(-c2ccccc2CCOC)c(CC(C)(C)CO)c2nc(Cl)ccc21]

Step 1. A mixture of 5-chloro-1H-pyrrolo [3,2-b]pyridine-3-carbaldehyde (8.5 g, 47.1 mmol) and ethyl 2-(triphenylphosphoranylidene) propionate (2.56 g, 70.7 mmol) in 1,4-dioxane (120 mL) was stirred at reflux for 4 h, then concentrated under reduced pressure. EtOAc (200 mL) was added and the mixture was washed with brine, dried over Na₂SO₄, and filtered. The filtrate was concentrated under reduced pressure and the residue was purified by silica gel column chromatography to give ethyl (E)-3-(5-chloro-1H-pyrrolo [3,2-b]pyridin-3-yl)-2-methylacrylate (7.5 g, 60% yield) as a solid. LCMS (ESI): m/z [M+H] calc'd for C₁₃H₁₃ClN₂O₂ 264.1; found 265.1.

Step 2. To a mixture of ethyl (E)-3-(5-chloro-1H-pyrrolo [3,2-b]pyridin-3-yl)-2-methylacrylate (7.5 g, 28.3 mmol) and NiCl₂ (4.8 g, 28.3 mmol) in 1:1 THF/MeOH (300 mL) was added NaBH₄ (21.5 g, 566 mmol) in 20 portions every 25 minutes. After complete addition, the mixture was stirred at rt for 30 min, then diluted with EtOAc (500 mL) and washed with brine, dried over Na₂SO₄, and filtered. The filtrate was concentrated under reduced pressure and the residue was purified by silica gel column chromatography to give ethyl 3-(5-chloro-1H-pyrrolo [3,2-b]pyridin-3-yl)-2-methylpropanoate (3.4 g, 45% yield) as a solid. LCMS (ESI): m/z [M+H] calc'd for C₁₃H₁₅ClN₂O₂ 266.1; found 267.1.

Step 3. To a mixture of ethyl 3-(5-chloro-1H-pyrrolo [3,2-b]pyridin-3-yl)-2-methylpropanoate (7.0 g, 26.2 mmol) and AgOTf (6.7 g, 26.2 mmol) in THF (50 mL) at 0° C. was added 12 (6.65 g, 26.2 mol). The mixture was stirred at 0° C. for 30 min then diluted with EtOAc (100 mL), washed with Na₂SO₃ (50 mL), brine (50 mL), dried over Na₂SO₄, and filtered. The filtrate was concentrated under reduced pressure and the residue was purified by silica gel column chromatography to give ethyl 3-(5-chloro-2-iodo-1H-pyrrolo [3,2-b]pyridin-3-yl)-2-methylpropanoate (6 g, 58% yield) as white solid. LCMS (ESI): m/z [M+H] calc'd for C₁₃H₁₄ClIN₂O₂ 392.0; found 393.0.

Step 4. To a mixture of ethyl 3-(5-chloro-2-iodo-1H-pyrrolo [3,2-b]pyridin-3-yl)-2-methylpropanoate (6.0 g, 15.3 mmol) and 2-(2-(2-methoxyethyl)phenyl)-4,4,5,5-tetramethyl-1,3,2-dioxaborolane (5.6 g, 21.4 mmol) and K₂CO₃ (6.3 g, 45.9 mmol) in 1,4-dioxane (150 mL) and H₂O (30 mL) under an atmosphere of N₂ was added Pd(dppf)Cl₂·DCM (1.3 g, 3.1 mmol). The mixture was heated to 80° C. and stirred for 4 h, then diluted with EtOAc (500 mL), washed with brine, dried over Na₂SO₄, and filtered. The filtrate was concentrated under reduced pressure and the residue was purified by silica gel column chromatography to give 3-(5-chloro-2-(2-(2-methoxyethyl)phenyl)-1H-pyrrolo [3,2-b]pyridin-3-yl)-2-methylpropanoate (5.5 g, 50% yield) as a viscous oil. LCMS (ESI): m/z [M+H] calc'd for C₂₂H₂₅ClN₂O₃ 400.2; found 401.2.

Step 5. A mixture of ethyl 3-(5-chloro-2-(2-(2-methoxyethyl)phenyl)-1H-pyrrolo [3,2-b]pyridin-3-yl)-2-methylpropanoate (5.5 g, 13.8 mmol), Cs₂CO₃ (8.9 g, 27.5 mmol), and EtI (3.5 g, 27.5 mmol) in DMF (30 mL) at rt was stirred for 10 h. The mixture was diluted with EtOAc (100 mL), washed with brine (20 mL×4), dried over Na₂SO₄, filtered, and concentrated in vacuo. The filtrate was concentrated under reduced pressure and the residue was purified by silica gel column chromatography to give ethyl 3-(5-chloro-1-ethyl-2-(2-(2-methoxyethyl)phenyl)-1H-pyrrolo [3,2-b]pyridin-3-yl)-2-methylpropanoate (5.6 g, 95% yield) as a viscous oil. LCMS (ESI): m/z [M+H] calc'd for C₂₅H₃₁ClN₂O₃ 428.2; found 429.2.

Step 6. To a mixture of ethyl 3-(5-chloro-1-ethyl-2-(2-(2-methoxyethyl)phenyl)-1H-pyrrolo [3,2-b]pyridin-3-yl)-2-methylpropanoate (5.4 g, 12.6 mmol) in THF (50 mL) at −65° C. was added 2M LDA (25 mL, 50 mmol) and stirred at −65° C. for 1 h. Mel (3.6 g, 25 mmol) was added and the mixture was stirred at −65° C. for 2.5 h, then aqueous NH₄Cl and EtOAc (50 mL) were added. The aqueous and organic layers were separated and the organic layer was washed with brine (30 mL), dried over Na₂SO₄, and filtered. The filtrate was concentrated under reduced pressure and the residue was purified by silica gel column chromatography to give ethyl 3-(5-chloro-1-ethyl-2-(2-(2-methoxyethyl)phenyl)-1H-pyrrolo [3,2-b]pyridin-3-yl)-2,2-dimethylpropanoate (3.2 g, 57% yield) as a viscous oil. LCMS (ESI): m/z [M+H] calc'd for C₂₅H₃₁ClN₂O₃ 442.2; found 443.2.

Step 7. To a mixture of ethyl 3-(5-chloro-1-ethyl-2-(2-(2-methoxyethyl)phenyl)-1H-pyrrolo [3,2-b]pyridin-3-yl)-2,2-dimethylpropanoate (1.0 g, 2.3 mmol) in THF (10 mL) at 5° C. was added LiBH₄ (196 mg, 9.0 mmol). The mixture was heated to 65° C. and stirred for 2 h then aqueous NH₄Cl and EtOAc (50 mL) added. The aqueous and organic layers were separated and the organic layer was washed with brine (30 mL), dried over Na₂SO₄, and filtered. The filtrate was concentrated under reduced pressure and the residue was purified by silica gel column chromatography to give 3-(5-chloro-1-ethyl-2-(2-(2-methoxyethyl)phenyl)-1H-pyrrolo [3,2-b]pyridin-3-yl)-2,2-dimethylpropan-1-ol (0.75 g, 82% yield) as a solid. LCMS (ESI): m/z [M+H] calc'd for C₂₃H₂₉ClN₂O₂ 400.2; found 401.2.

### Intermediate 11: Methyl (3S)-1-{(2S)-2-(tert-butoxycarbonyl) amino-3-[3-fluoro-5-(4,4,5,5-tetramethyl-1,3,2-dioxaborolan-2-yl)phenyl]propanoyl}-1,2-diazinane-3-carboxylate

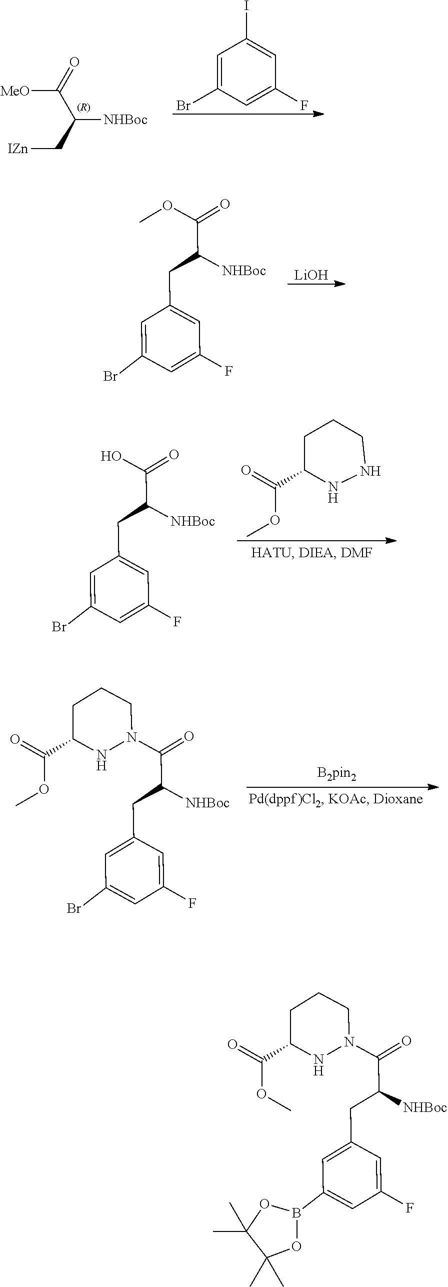

Step 1. To a stirred solution of methyl (2R)-2-{[(tert-butoxy) carbonyl]amino}-3-(iodozincio) propanoate (12 g, 30 mmol, 1.2 eq) in DMF (100 mL) was added 1-bromo-3-fluoro-5-iodobenzene (7.5 g, 25 mmol, 1 eq) and Pd(PPh₃)₂Cl₂ (1.7 g, 2.5 mmol, 0.1 equiv) at 20° C. under N₂ atmosphere. The resulting mixture was stirred for 2 hrs at 65° C. under N₂ atmosphere. The reaction mixture was quenched with water and extracted with EA (200 mL×2). The organic phase was washed with water (200 mL×1) and brine (100 mL×1) and concentrated to dryness to give a residue. The residue was purified by prep-TLC (PE/EA=10/1) to afford methyl 3-(3-bromo-5-fluorophenyl)-2-{[(tert-butoxy) carbonyl]amino}propanoate (6 g, 58% yield) as a colorless oil. LCMS (ESI) m/z=398.1 [M+Na]⁺, calculated for C₁₅H₁₉BrFNO₄: 375.0

Step 2. To a solution of methyl 3-(3-bromo-5-fluorophenyl)-2-{[(tert-butoxy) carbonyl]amino}propanoate (3.2 g, 8.5 mmol, 1 eq) in THF (50 mL) was added Lithium hydroxide (610.7 mg, 25.5 mmol, 3 eq) in H₂O (10 mL). Then the reaction mixture was stirred at 20° C. for 1 h. The mixture was adjusted to pH=5.0 with 1 M HCl aqueous solution. The mixture was quenched with H₂O (150 mL) and extracted with EA (200 mL×3). The combined organic layers was washed bine (50 mL), dried over Na₂SO₄ and concentrated to afford 3-(3-bromo-5-fluorophenyl)-2-{[(tert-butoxy) carbonyl]amino}propanoic acid (2.65 g, 68% yield) as a white solid. LCMS (ESI) m/z=384.1 [M+Na]⁺, calculated for C₁₄H₁₅BrFNO₄ MW: 361.0.

Step 3. To a mixture of 3-(3-bromo-5-fluorophenyl)-2-{[(tert-butoxy) carbonyl]amino}propanoic acid (2.3 g, 6.4 mmol, 1 eq) and methyl (3S)-1,2-diazinane-3-carboxylate (1.66 g, 11.5 mmol, 1.8 eq) in DMF (150 mL) was added HATU (4.9 g, 12.8 mmol, 2 eq) and DIEA (16.5 g, 128 mmol, 20 eq) in DMF (50 mL) at 0° C. Then the reaction mixture was stirred at 0° C. for 1 h. The mixture was quenched with H₂O (100 mL) and extracted with EA (300 ml×3). The combined organic layers was washed bine (50 mL), dried over Na₂SO₄ and concentrated to give the residue, which was purified by Pre-HPLC eluting with acetonitrile in water (0.1% FA) from 60% to 70% in 10 minutes to give methyl (3S)-1-[(2S)-3-(3-bromo-5-fluorophenyl)-2-{[(tert-butoxy) carbonyl]amino}propanoyl]-1,2-diazinane-3-carboxylate (2.7 g, 78% yield) as a pale yellow solid. LCMS (ESI) m/z=510.1 [M+Na]⁺,calculated for C₂₀H₂₇BrFNO₅: 487.1.

Step 4. A mixture of methyl(S)-1-((S)-3-(3-bromo-5-fluorophenyl)-2-((tert-butoxycarbonyl) amino) propanoyl) hexahydropyridazine-3-carboxylate (3 g, 6.16 mmol, 1 eq), 4,4,5,5-tetramethyl-2-(4,4,5,5-tetramethyl-1,3,2-dioxaborolan-2-yl)-1,3,2-dioxaborolane (1.9 g, 7.4 mmol, 1.2 eq), KOAc (900 mg, 9.24 mmol, 1.5 eq) and Pd(dppf)Cl₂DCM (0.3 g, 0.37 mmol, 0.05 eq) in dioxane (50 mL) was heated at 100° C. for 17 h under N₂ atmosphere. The mixture was concentrated and purified by column chromatography (DCM/MeOH=100/1 to 40/1) to give methyl (3S)-1-(2S)-2-{(tert-butoxycarbonyl) amino-3-[3-fluoro-5-(4,4,5,5-tetramethyl-1,3,2-dioxaborolan-2-yl)phenyl]propanoyl}-1,2-diazinane-3-carboxylate (2.6 g, 79% yield) as a yellow oil. LCMS (ESI) m/z=536.2 [M+H]⁺, calculated for C₂₆H₃₉BFNO₇: 535.3.

Compounds A341 and A342 may be prepared using methods disclosed herein via Intermediate 11.

### Example A75. Synthesis of two atropisomers of (2S)—N-[(8S,14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-3-methyl-2-{N-methyl-1-[(3S)-1-(prop-2-enoyl) pyrrolidin-3-yl]formamido}butanamide

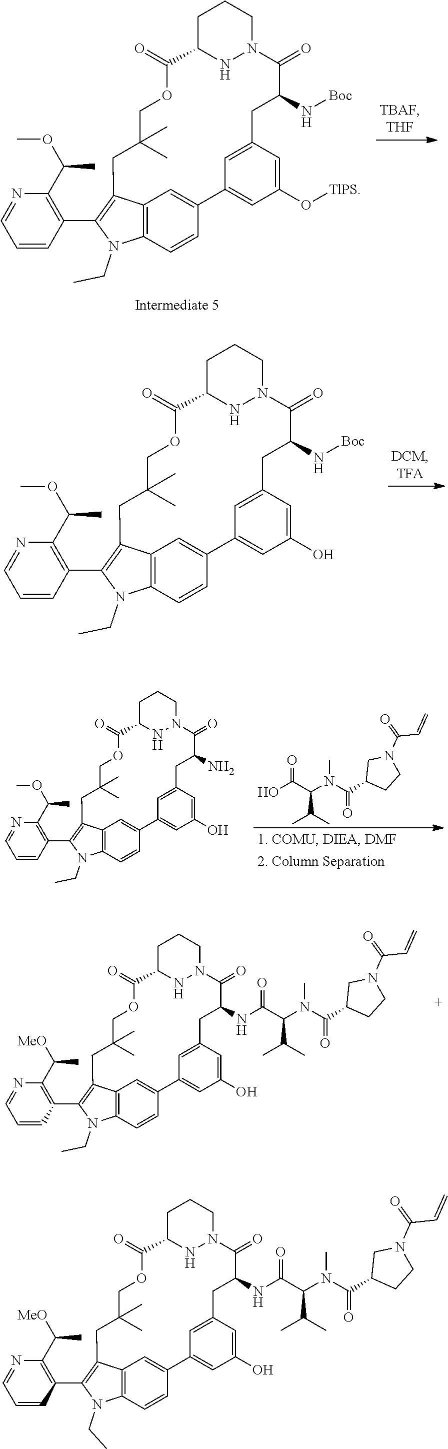

Step 1. To a stirred mixture of tert-butyl ((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (18.0 g, 20.1 mmol) in THF (180 mL) at 0° C. was added a 1M solution of TBAF in THF (24.1 mL, 24.1 mmol). The mixture was stirred at 0° C. for 1 h, then diluted with brine (1.5 L) and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give tert-butyl ((6³S,4S)-1¹-ethyl-2⁵-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (11.5 g, 69% yield) as a solid. LCMS (ESI): m/z [M+H] calc'd for C₄₂H₅₃N₅O₇ 739.4; found 740.4.

Step 2. To a stirred mixture of tert-butyl ((6³S,4S)-1¹-ethyl-2⁵-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (11.5 g, 15.5 mmol) in DCM (120 mL) at 0° C. was added TFA (60 mL, 808 mmol). The mixture was stirred at 0° C. for 1 h, then concentrated under reduced pressure and the residue again concentrated under reduced pressure with toluene (20 mL; repeated ×3) to give (6³S,4S)-4-amino-1¹-ethyl-2⁵-hydroxy-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (12 g, crude), which was used directly in the next step without further purification. LCMS (ESI): m/z [M+H] calc'd for C₃₇H₄₅N₅O₅ 639.3; found 640.6.

Step 3. To a stirred mixture of (6³S,4S)-4-amino-1¹-ethyl-2⁵-hydroxy-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (11.9 g, 18.6 mmol) in DMF (240 mL) at 0° C. under an atmosphere of N₂ was added DIPEA (48.1 g, 372 mmol), (2S)-3-methyl-2-[N-methyl-1-[(3S)-1-(prop-2-enoyl) pyrrolidin-3-yl]formamido]butanoic acid (9.45 g, 33.5 mmol) and COMU (11.95 g, 27.9 mmol) in portions. The mixture was stirred ay 0° C. for 90 min, then diluted with brine (1.5 L) and extracted with EtOAc (3×1 L). The combined organic layers were washed with brine (2×500 mL), dried over anhydrous Na₂SO₄, and filtered. After filtration, the filtrate was concentrated under reduced pressure. The filtrate was concentrated under reduced pressure and the residue was purified by silica gel column chromatography (×2) to give two atropisomers of (2S)—N-[(8S, 14S,20M)-22-ethyl-4-hydroxy-21-{2-[(1S)-1-methoxyethyl]pyridin-3-yl}-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-3-methyl-2-{N-methyl-1-[(3S)-1-(prop-2-enoyl) pyrrolidin-3-yl]formamido}butanamide (2.7 g, 15.5%, yield) and (4.2 g, 24.7% yield) both as solids. LCMS (ESI): m/z [M+H] calc'd for C₅₁H₆₅N₇O₈ 903.5; found 904.7; ¹H NMR (400 MHZ, DMSO-d₆) δ 9.35-9.27 (m, 1H), 8.77 (dd, J=4.7, 1.7 Hz, 1H), 7.95 (dq, J=6.2, 2.0 Hz, 2H), 7.55 (ddd, J=28.0, 8.2, 4.3 Hz, 3H), 7.08 (dd, J=37.9, 6.2 Hz, 2H), 6.69-6.48 (m, 2H), 6.17 (ddt, J=16.7, 7.2, 2.3 Hz, 1H), 5.74-5.62 (m, 1H), 5.43-5.34 (m, 1H), 5.12-5.00 (m, 1H), 4.25 (d, J=12.3 Hz, 1H), 4.17-3.99 (m, 3H), 3.89-3.65 (m, 4H), 3.66-3.45 (m, 3H), 3.12 (s, 4H), 2.95-2.70 (m, 6H), 2.41-2.06 (m, 5H), 1.99-1.88 (m, 1H), 1.82 (d, J=12.1 Hz, 2H), 1.54 (t, J=12.0 Hz, 1H), 1.21 (dd, J=6.3, 2.5 Hz, 3H), 1.11 (t, J=7.1 Hz, 3H), 0.99-0.88 (m, 6H), 0.79 (ddd, J=27.8, 6.7, 2.1 Hz, 3H), 0.48 (d, J=3.7 Hz, 3H) and LCMS (ESI): m/z [M+H] calc'd for C₅₁H₆₅N₇O₈ 903.5; found 904.7; ¹H NMR (400 MHZ, DMSO-d₆) δ 9.34-9.27 (m, 1H), 8.77 (dd, J=4.7, 1.7 Hz, 1H), 8.17-7.77 (m, 3H), 7.64-7.43 (m, 3H), 7.33 (d, J=13.7 Hz, 1H), 7.05-6.94 (m, 1H), 6.69-6.41 (m, 2H), 6.26-5.94 (m, 1H), 5.73-5.63 (m, 1H), 5.50-5.20 (m, 2H), 4.40-4.15 (m, 3H), 4.00-3.40 (m, 9H), 3.11 (d, J=4.4 Hz, 3H), 2.93-2.60 (m, 8H), 2.29-2.01 (m, 3H), 1.99 (s, 1H), 1.87-1.75 (m, 2H), 1.73-1.47 (m, 2H), 1.40 (d, J=6.0 Hz, 3H), 1.01-0.88 (m, 6H), 0.85-0.65 (m, 7H), 0.56 (s, 3H).

### Example A89. Synthesis of (2S)—N-[(8S,14S)-22-ethyl-4-hydroxy-18, 18-dimethyl-21-[6-(4-methylpiperazin-1-yl) pyridin-3-yl]-9,15-dioxo-16-oxa-10,22,28-triazapentacyclo [18.5.2.1^{2,6}.1^{10,14}.0^{23,27}]nonacosa-1 (26), 2,4,6 (29), 20,23 (27),24-heptaen-8-yl]-3-methyl-2-{N-methyl-1-[(3S)-1-(prop-2-enoyl) pyrrolidin-3-yl]formamido}butanamide

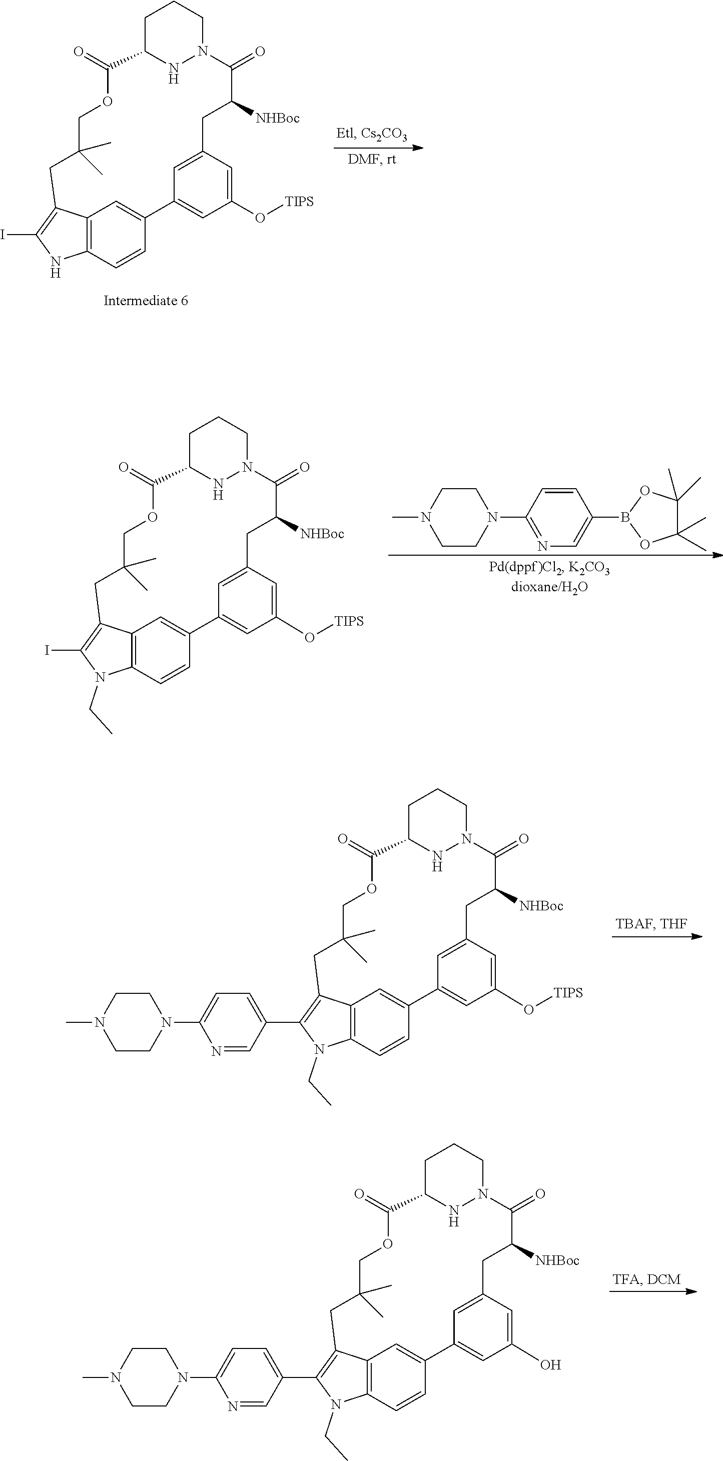

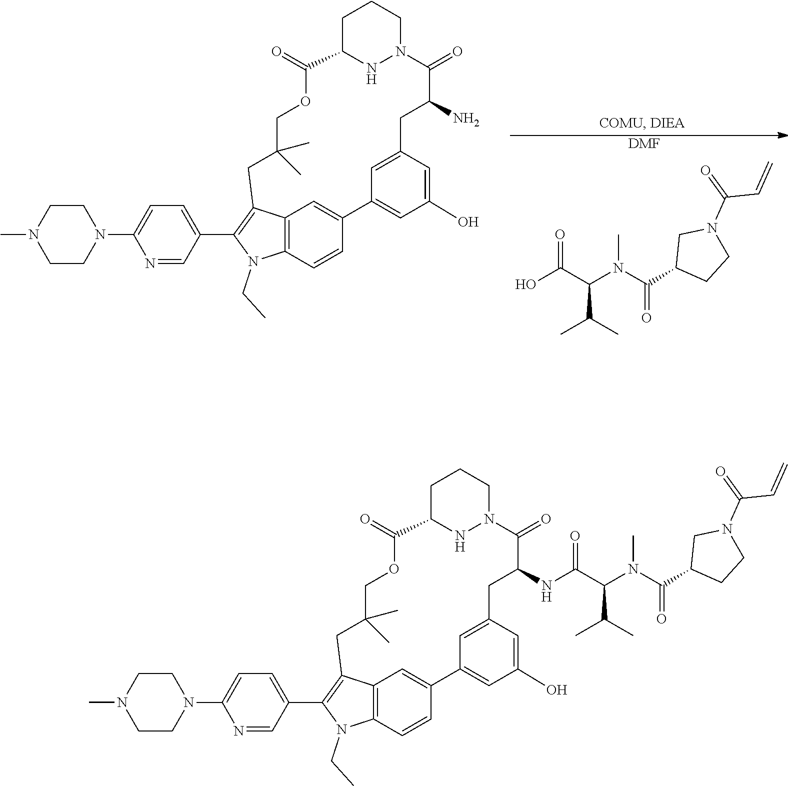

Step 1. To a mixture of tert-butyl ((6³S,4S)-1²-iodo-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (240.00 mg, 0.279 mmol, 1.00 equiv) and Cs₂CO₃ (182 mg, 0.558 mmol, 2 equiv) in DMF (5.00 mL) was added ethyl iodide (113.45 mg, 0.727 mmol, 2.60 equiv) dropwise at 0° C. The reaction was stirred for 16 h at 25° C. The resulting mixture was diluted with water (10 mL) and extracted with EtOAc (3×10 mL). The combined organic layers were washed with brine (3×10 mL), and dried over anhydrous Na₂SO₄. The filtrate was concentrated under reduced pressure and the remaining residue was purified by silica gel column chromatography to afford tert-butyl ((6³S,4S)-11-ethyl-12-iodo-10, 10-dimethyl-5,7-dioxo-25-((triisopropylsilyl)oxy)-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (190 mg, 77% yield) as a yellow solid.

Step 2. A mixture of tert-butyl ((6³S,4S)-11-ethyl-12-iodo-10,10-dimethyl-5,7-dioxo-25-((triisopropylsilyl)oxy)-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (500 mg, 0.54 mmol), 1-methyl-4-[5-(4,4,5,5-tetramethyl-1,3,2-dioxaborolan-2-yl) pyridin-2-yl]piperazine (257 mg, 0.8 mmol), Pd(dppf)Cl2 (83 mg, 0.11 mmol) and K₂CO₃ (156 mg, 1.1 mmol) in 1,4-dioxane (25 mL) and H₂O (5 mL) under an atmosphere of Ar was stirred at 80° C. for 2 h. The mixture was concentrated under reduced pressure and the residue was purified by prep-TLC to afford tert-butyl ((6³S,4S)-1¹-ethyl-10,10-dimethyl-1²-(6-(4-methylpiperazin-1-yl) pyridin-3-yl)-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (400 mg, 76% yield) as a solid. LCMS (ESI): m/z [M+H] calc'd for C₅₃H₇₇N₇O₆Si 935.6; found 936.6.

Step 3. A mixture of tert-butyl ((6³S,4S)-1¹-ethyl-10,10-dimethyl-1²-(6-(4-methylpiperazin-1-yl) pyridin-3-yl)-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (350 mg, 0.36 mmol) and 1M TBAF in THF (0.4 mL, 0.4 mmol) in THF (5 mL) was stirred at 0° C. for 1 h. The mixture was concentrated under reduced pressure and the residue was purified by silica gel column chromatography to give tert-butyl ((6³S,4S)-1¹-ethyl-2⁵-hydroxy-10, 10-dimethyl-12-(6-(4-methylpiperazin-1-yl) pyridin-3-yl)-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (290 mg, 100% yield) as a solid. LCMS (ESI): m/z [M+H] calc'd for C₄₄H₅₇N₇O₆ 779.4; found 780.4.

Step 4. A mixture of tert-butyl ((6³S,4S)-11-ethyl-25-hydroxy-10, 10-dimethyl-12-(6-(4-methylpiperazin-1-yl) pyridin-3-yl)-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (300 mg, 0.37 mmol) in TFA (5 mL) and DCM (5 mL) was stirred at rt for 1 h. The mixture was concentrated under reduced pressure to give (6³S,4S)-4-amino-11-ethyl-25-hydroxy-10, 10-dimethyl-12-(6-(4-methylpiperazin-1-yl) pyridin-3-yl)-6¹,6²,6³,6⁴,6⁵,6⁶-hexahydro-11H-8-oxa-1 (5,3)-indola-6 (1,3)-pyridazina-2 (1,3)-benzenacycloundecaphane-5,7-dione (300 mg, crude) as a solid. LCMS (ESI): m/z [M+H] calc'd for C₃₉H₄₉N₇O₄ 679.4; found 680.3.

Step 5. To a mixture of (6³S,4S)-4-amino-11-ethyl-25-hydroxy-10, 10-dimethyl-12-(6-(4-methylpiperazin-1-yl) pyridin-3-yl)-6¹,6²,6³,6⁴,6⁵,6⁶-hexahydro-11H-8-oxa-1 (5,3)-indola-6 (1,3)-pyridazina-2 (1,3)-benzenacycloundecaphane-5,7-dione (300 mg, 0.36 mmol) in DMF (3 mL) at 0° C. under an atmosphere of N₂ was added DIPEA (0.96 mL, 5.4 mmol) and (2S)-3-methyl-2-[N-methyl-1-[(3S)-1-(prop-2-enoyl) pyrrolidin-3-yl]formamido]butanoic acid (213 mg, 0.72 mmol), followed by dropwise addition of COMU (243 mg, 0.56 mmol). H₂O was added at 0° C. and the mixture was extracted with EtOAc (3×10 mL). The combined organic layers were washed with brine (3×10 mL), dried over anhydrous Na₂SO₄, and filtered. The filtrate was concentrated under reduced pressure and the crude residue was purified by Prep-HPLC to give (2S)—N-[(8S, 14S)-22-ethyl-4-hydroxy-18, 18-dimethyl-21-[6-(4-methylpiperazin-1-yl) pyridin-3-yl]-9, 15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methyl-2-{N-methyl-1-[(3S)-1-(prop-2-enoyl) pyrrolidin-3-yl]formamido}butanamide (45 mg, 13.2% yield) as a solid. LCMS (ESI): m/z [M+H] calc'd for C₅₃H₆₉N₉O₇ 943.5; found 944.8; ¹H NMR (400 MHz, DMSO-d₆) δ 9.39-9.23 (m, 1H), 8.64-8.60 (m, 1H), 8.19-8.16 (m, 1H), 8.15 (d, J=6.2 Hz, 1H), 7.86 (s, 1H), 7.66-7.62 (m, 1H), 7.56-7.54 (m, 1H), 7.50-7.43 (m, 1H), 7.13-7.11 (m, 1H), 7.03-6.95 (m, 1H), 6.70-6.47 (m, 2H), 6.17 (ddt, J=16.8, 6.4, 2.8 Hz, 1H), 5.76-5.63 (m, 1H), 5.45-5.33 (m, 1H), 5.11 (m, 1H), 4.75-4.72 (m, 1H), 4.28-4.24 (m, 1H), 4.11-3.98 (m, 4H), 3.91-3.76 (m, 1H), 3.73-3.71 (m, 1H), 3.59-3.56 (m, 7H), 3.51-3.40 (m, 2H), 3.08-2.94 (m, 1H), 2.94-2.92 (m, 2H), 2.92-2.87 (m, 2H), 2.86-2.83 (m, 2H), 2.80-2.65 (m, 2H), 2.83-2.82 (m, 3H), 2.28-2.25 (m, 3H), 2.08-2.05 (m, 2H), 2.02-1.96 (m, 1H), 1.87-1.78 (m, 1H), 1.74-1.66 (m, 1H), 1.56-1.48 (m, 1H), 1.11-1.08 (m, 4H), 0.99-0.92 (m, 2H), 0.89-0.87 (m, 5H), 0.82-0.73 (m, 2H).

### Example A115. Synthesis of two atropisomers of (2S)—N-[(8S,14S,20P)-22-ethyl-21-{4-[(1S)-1-methoxyethyl]pyridin-3-yl}-18, 18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-3-methyl-2-{N-methyl-1-[(3S)-1-(prop-2-enoyl) pyrrolidin-3-yl]formamido}butanamide

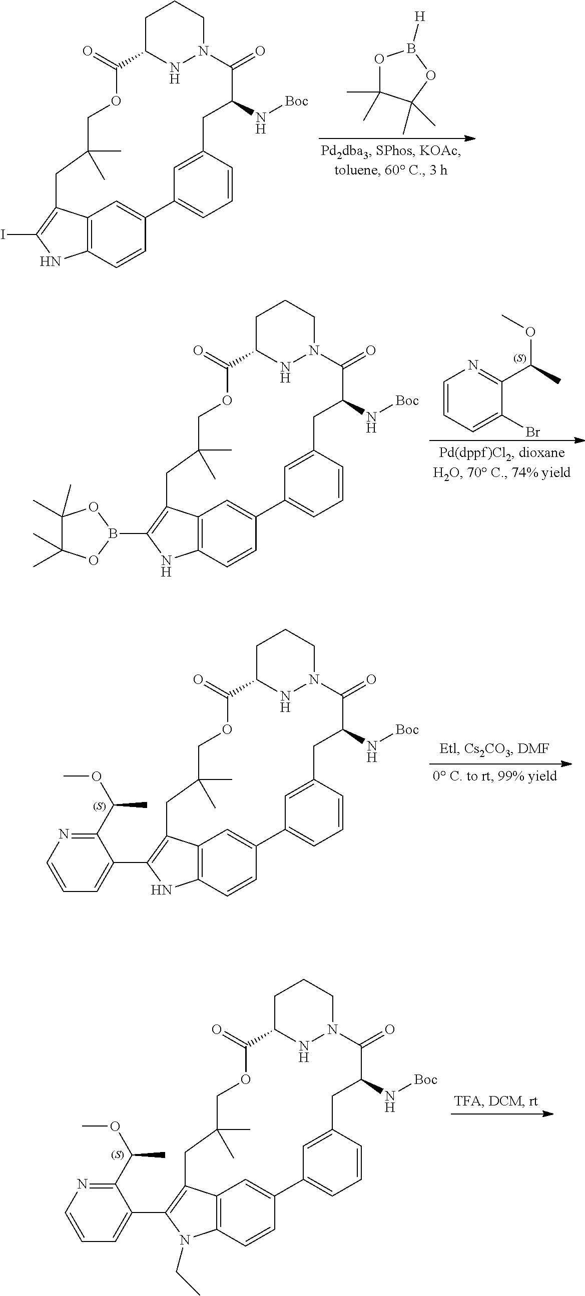

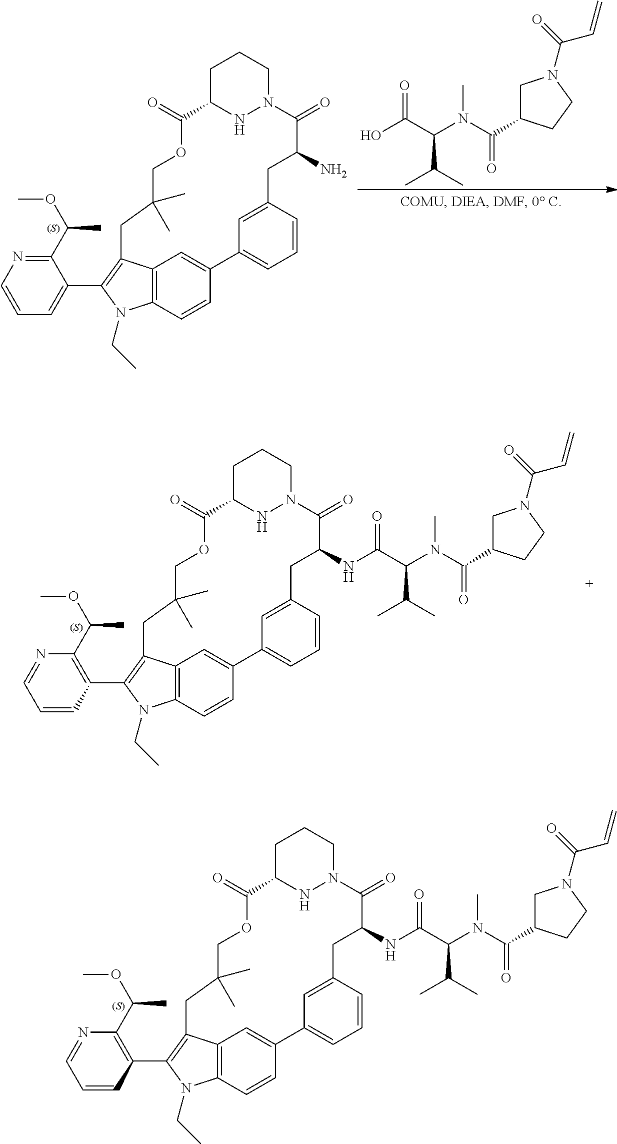

Step 1. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (22.00 g, 32.042 mmol, 1.00 equiv), toluene (300.00 mL), Pd₂ (dba) 3 (3.52 g, 3.845 mmol, 0.12 equiv), S-Phos (3.95 g, 9.613 mmol, 0.30 equiv), and KOAc (9.43 g, 96.127 mmol, 3.00 equiv) at room temperature. To the mixture was added 4,4,5,5-tetramethyl-1,3,2-dioxaborolane (26.66 g, 208.275 mmol, 6.50 equiv) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford tert-butyl ((6³S,4S)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (22 g, 90%) as a light yellow solid. ESI-MS m/z=687.3 [M+H]⁺; Calculated MW: 686.4.

Step 2. A mixture of tert-butyl ((6³S,4S)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (2.0 g, 2.8 mmol), 3-bromo-2-[(1S)-1-methoxyethyl]pyridine (0.60 g, 2.8 mmol), Pd(dppf)Cl₂ (0.39 g, 0.5 mmol), and K₃PO₄ (1.2 g, 6.0 mmol) in 1,4-dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.5 g, 74% yield) as a solid. LCMS (ESI): m/z [M+H] calc'd for C₄₀H₄₉N₅O₆ 695.4; found 696.5.

Step 3. A mixture of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.5 g, 2.1 mmol), Cs²CO₃ (2.1 g, 6.3 mmol), and ethyl iodide (0.43 mL, 5.1 mmol) in DMF (50 mL) was stirred at 0° C. for 16 h. The mixture was quenched at 0° C. with H₂O and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.5 g, 99% yield) as a solid. LCMS (ESI): m/z [M+H] calc'd for C₄₂H₅₃N₅O₆ 723.4; found 724.6.

Step 4. A mixture of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.3 g, 1.7 mmol) in TFA (10 mL) and DCM (20 mL) was stirred at 0° C. for 2 h. The mixture was concentrated under reduced pressure to afford (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (1.30 g, crude) as a solid. LCMS (ESI): m/z [M+H] calc'd for C₃₇H₄₅N₅O₄ 623.3; found 624.4.

Step 5. Into a 40-mL vial purged and maintained with an inert atmosphere of Ar, was placed (6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (250 mg, 0.4 mmol), (2S)-3-methyl-2-[N-methyl-1-[(3S)-1-(prop-2-enoyl) pyrrolidin-3-yl]formamido]butanoic acid (226 mg, 0.8 mmol), DIPEA (774 mg, 6.0 mmol), and DMF (3 mL). A solution of COMU (257 mg, 0.6 mmol) in DMF (2 mL) was added at 0° C. and the resulting mixture was stirred at 0° C. for 1 h. The mixture was filtered, the filtrate was concentrated under reduced pressure, and the residue was purified by prep-HPLC to give two atropisomers of (2S)—N-[(8S,14S,20P)-22-ethyl-21-{4-[(1S)-1-methoxyethyl]pyridin-3-yl}-18, 18-dimethyl-9,15-dioxo-16-oxa-10,22,28-triazapentacyclo [18.5.2.1^{2,6}.1^{10,14}.0^{23,27}]nonacosa-1 (26), 2,4,6 (29),20,23 (27),24-heptaen-8-yl]-3-methyl-2-{N-methyl-1-[(3S)-1-(prop-2-enoyl) pyrrolidin-3-yl]formamido}butanamide (56 mg, 15% yield) and (46 mg, 13% yield) both as a solid. LCMS (ESI): m/z [M+H] calc'd for C₅₁H₆₅N₇O₇ 887.5; found 888.4; ¹H NMR (400 MHZ, DMSO-d₆) δ 8.81 (s, 1H), 8.07 (s, 1H), 8.05-7.96 (m, 1H), 7.78-7.45 (m, 5H), 7.41-7.08 (m, 2H), 6.66-6.58 (m, 1H), 6.18 (d, J=17.0 Hz, 1H), 5.75-5.67 (m, 1H), 5.46-5.31 (m, 1H), 5.16-5.04 (m, 1H), 4.75 (dd, J=10.9, 4.5 Hz, 1H), 4.31-4.21 (m, 2H), 4.11-3.95 (m, 3H), 3.87-3.71 (m, 5H), 3.74-3.54 (m, 3H), 3.11 (s, 4H), 2.95 (d, J=9.7 Hz, 2H), 2.85-2.72 (m, 3H), 2.31-2.04 (m, 3H), 1.88-1.47 (m, 2H), 1.24-1.21 (m, 3H), 1.16-1.08 (m, 3H), 1.03-0.91 (m, 6H), 0.85-0.74 (m, 3H), 0.51-0.46 (m, 3H) and LCMS (ESI): m/z [M+H] calc'd for C₅₁H₆₅N₇O₇ 887.5; found 888.4; ¹H NMR (400 MHZ, DMSO-d₆) δ 8.77 (s, 1H), 8.71-8.63 (m, 0.5H), 8.23-8.17 (m, 0.5H), 8.00 (s, 1H), 7.85 (t, J=9.9 Hz, 2H), 7.77-7.62 (m, 3H), 7.57-7.50 (m, 1H), 7.33-7.22 (m, 1H), 7.15-7.06 (m, 1H), 6.73-6.56 (m, 1H), 6.17 (ddd, J=16.7, 6.1, 2.7 Hz, 1H), 5.76-5.64 (m, 1H), 5.49-5.29 (m, 2H), 4.70 (dd, J=10.8, 3.5 Hz, 1H), 4.33-4.22 (m, 3H), 4.14-3.95 (m, 2H), 3.86-3.77 (m, 1H), 3.72-3.65 (m, 2H), 3.61 (t, J=10.6 Hz, 3H), 3.46-3.42 (m, 1H), 3.13 (d, J=4.8 Hz, 3H), 2.99 (d, J=14.4 Hz, 1H), 2.95-2.70 (m, 6H), 2.24-1.99 (m, 4H), 1.95-1.44 (m, 4H), 1.40 (d, J=6.1 Hz, 3H), 0.98-0.87 (m, 6H), 0.86-0.64 (m, 6H), 0.64-0.54 (m, 3H).

### Example A2. Synthesis of (2S)—N-[(8S,14S)-4-amino-22-ethyl-21-[2-(2-methoxyethyl)phenyl]-18,18-dimethyl-9,15-dioxo-16-oxa-10,22,28-triazapentacyclo [18.5.2.1^{2,6}.1^{10,1}4.0^{23,27}]nonacosa-1 (26), 2,4,6 (29),20,23 (27),24-heptaen-8-yl]-3-methyl-2-{N-methyl-1-[(3S)-1-(prop-2-enoyl) pyrrolidin-3-yl]formamido}butanamide

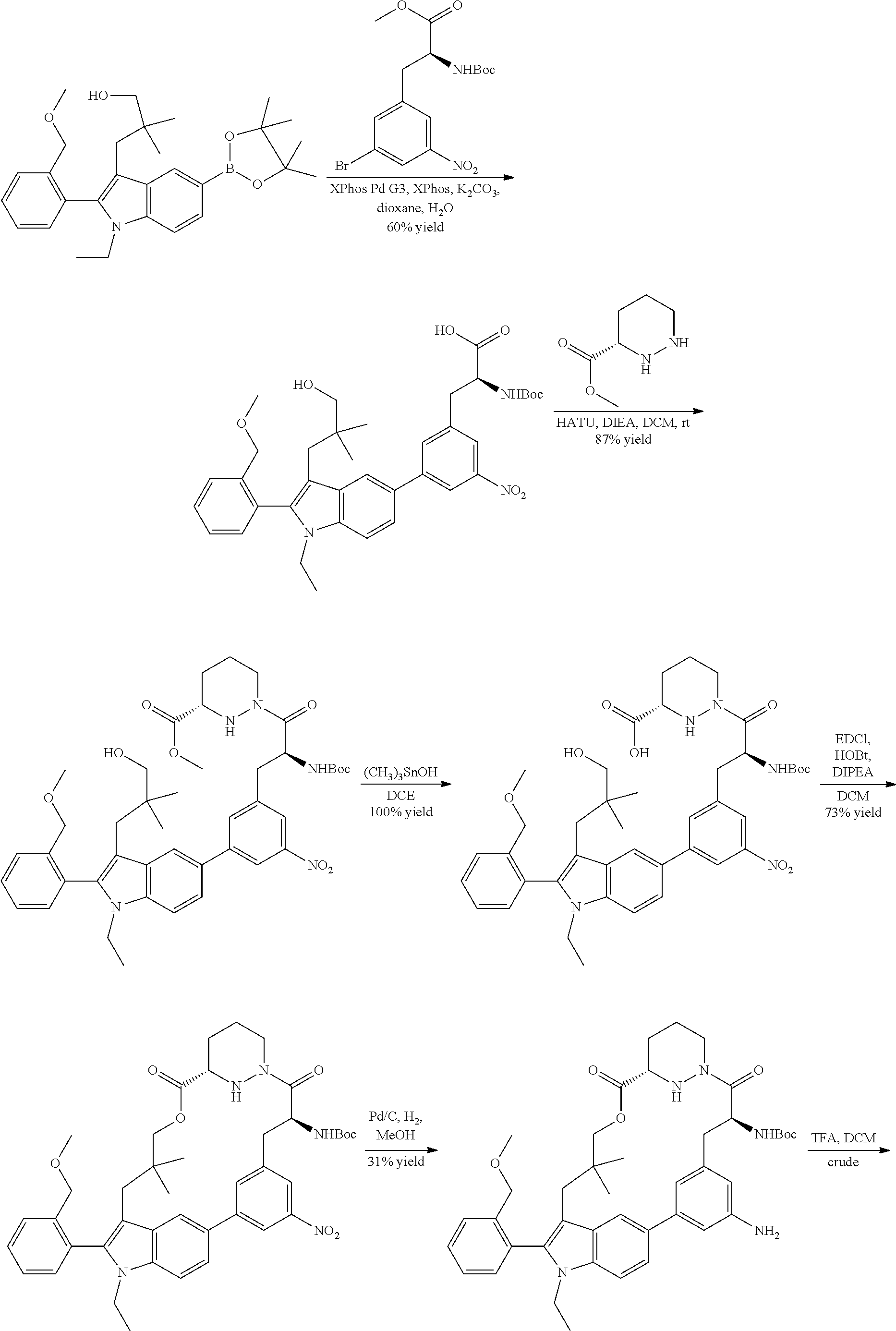

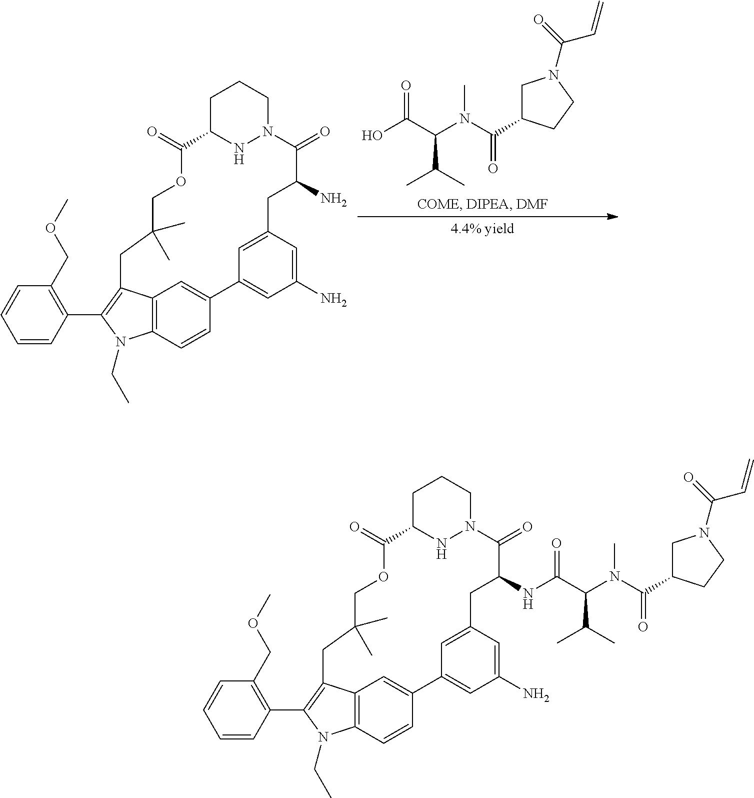 [SMILES: CCn1c(-c2ccccc2COC)c2c3cc(ccc31)-c1cc(N)cc(c1)C[C@H](N)C(=O)N1CCC[C@H](N1)C(=O)OCC(C)(C)C2 ; C=CC(=O)N1CC[C@H](C(=O)N(C)[C@H](C(=O)O)C(C)C)C1 ; C=CC(=O)N1CC[C@H](C(=O)N(C)[C@H](C(=O)N[C@H]2Cc3cc(N)cc(c3)-c3ccc4c(c3)c(c(-c3ccccc3COC)n4CC)CC(C)(C)COC(=O)[C@@H]3CCCN(N3)C2=O)C(C)C)C1]

Step 1. Into a 25 mL sealed tube were added 3-[1-ethyl-2-[2-(methoxymethyl)phenyl]-5-(4,4,5,5-tetramethyl-1,3,2-dioxaborolan-2-yl) indol-3-yl]-2,2-dimethylpropan-1-ol (590 mg, 1.2 mmol), methyl (2S)-3-(3-bromo-5-nitrophenyl)-2-[(tert-butoxycarbonyl) amino]propanoate (747 mg, 1.9 mmol), XPhos Pd G3 (105 mg, 0.12 mmol), XPhos (71 mg, 0.15 mmol), K₂CO₃ (427 mg, 3.1 mmol), and 1,4-dioxane (2 mL) under an atmosphere of N₂ at rt. The mixture was heated to 60° C. and stirred overnight, then cooled and H₂O added. The mixture was extracted with EtOAc (3×20 mL) and the combined organic layers were washed with brine (1×20 mL), dried over anhydrous Na₂SO₄, and filtered. The filtrate was concentrated under reduced pressure and the residue was purified by silica gel column chromatography to give (2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoic acid (500 mg, 61% yield) as a solid. LCMS (ESI): m/z [M+H] calc'd for C₃₇H₄₅N₃O₈ 659.3; found 660.4.

Step 2. A mixture of (2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoic acid (500 mg, 0.79 mmol), methyl (3S)-1,2-diazinane-3-carboxylate (164 mg, 1.1 mmol), DCM (6 mL), DIPEA (294 mg, 2.3 mmol) and HATU (432 mg, 1.1 mmol) was stirred at 0° C. for 1 h under an atmosphere of air. H₂O was added and the mixture was extracted with DCM (3×20 mL), then the combined organic layers were dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylate (520 mg, 87% yield) as a solid. LCMS (ESI): m/z [M+H] calc'd for C₄₃H₅₅N₅O₉ 785.4; found 786.8.

Step 3. Into a 40 mL sealed tube were added methyl (3S)-1-[(2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylate (510 mg, 0.65 mmol), DCE (5 mL) and trimethyltin hydroxide (587 mg, 3.3 mmol) at rt under an atmosphere of air. The mixture was heated to 60° C. and stirred overnight, cooled, and diluted with DCM (20 mL). The mixture was washed with 0.1 N KHSO₄ (3×20 mL), dried over anhydrous Na₂SO₄, filtered, and the filtrate was concentrated under reduced pressure to give (3S)-1-[(2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylic acid (500 mg, 100%) as a solid. LCMS (ESI): m/z [M+H] calc'd for C₄₂H₅₃N₅O₉ 771.4; found 772.7.

Step 4. A mixture of (3S)-1-[(2S)-2-[(tert-butoxycarbonyl) amino]-3-[3-[1-ethyl-3-(3-hydroxy-2,2-dimethylpropyl)-2-[2-(methoxymethyl)phenyl]indol-5-yl]-5-nitrophenyl]propanoyl]-1,2-diazinane-3-carboxylic acid (490 mg, 0.64 mmol), DCM (100 mL), DIPEA (2.5 g, 19.0 mmol), HOBT (429 mg, 3.2 mmol), and EDCl (3.65 g, 19.0 mmol) at room temperature was stirred at rt overnight under an atmosphere of air. H₂O was added and the mixture was extracted with DCM (3×60 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-(methoxymethyl)phenyl)-10, 10-dimethyl-25-nitro-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (350 mg, 73% yield) as a solid. LCMS (ESI): m/z [M+H] calc'd for C₄₂H₅₁N₅O₈ 753.4; found 754.2.

Step 5. A mixture of tert-butyl ((6³S,4S)-1¹-ethyl-1²-(2-(methoxymethyl)phenyl)-10,10-dimethyl-2⁵-nitro-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (200 mg, 0.27 mmol), MeOH (4 mL), and Pd on carbon (20 mg) was stirred at rt for 2 h under an atmosphere of H₂. The mixture was filtered, the filter cake was washed with MeOH (3×5 mL), and the filtrate was concentrated under reduced pressure to give tert-butyl ((6³S,4S)-2⁵-amino-1¹-ethyl-1²-(2-(methoxymethyl)phenyl)-10,10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (60 mg, 31% yield) as a solid. LCMS (ESI): m/z [M+H] calc'd for C₄₂H₅₃N₅O₆ 723.4; found 724.4.

Step 6. Into an 8 mL vial were added tert-butyl ((6³S,4S)-2⁵-amino-1¹-ethyl-1²-(2-(methoxymethyl)phenyl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (50 mg, 0.07 mmol), DCM (1 mL), and TFA (158 mg, 1.4 mmol) at 0° C. under an atmosphere of air. The mixture was stirred for at 0° C. for 2 h then concentrated under reduced pressure to give (6³S,4S)-2⁵,4-diamino-1¹-ethyl-1²-(2-(methoxymethyl)phenyl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (45 mg) as a solid, which was used directly in the next step directly without further purification. LCMS (ESI): m/z [M+H] calc'd for C₃₇H₄₅N₅O₄ 623.3; found 624.4.

Step 7. Into an 8 mL vial were added (6³S,4S)-2⁵,4-diamino-1¹-ethyl-1²-(2-(methoxymethyl)phenyl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (40 mg, 0.06 mmol), DMF (1 mL), DIPEA (75 mg, 0.58 mmol), and COMU (41 mg, 0.1 mmol) at 0° C. under an atmosphere of air. The mixture was stirred at 0° C. for 1 h, then H₂O added. The mixture was extracted with EtOAc (3×30 mL), the combined organic layers were concentrated under reduced pressure, and purified by prep-HPLC to give (2S)—N-[(8S, 14S)-4-amino-22-ethyl-21-[2-(2-methoxyethyl)phenyl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-3-methyl-2-{N-methyl-1-[(3S)-1-(prop-2-enoyl) pyrrolidin-3-yl]formamido}butanamide (2.5 mg, 4.4% yield) as a solid. LCMS (ESI): m/z [M+H] calc'd for C₅₁H₆₅N₇O₇ 887.5; found 888.6; ¹H NMR (400 MHZ, DMSO-d₆) δ 8.74-8.55 (m, 1H), 7.89 (d, J=9.6 Hz, 1H), 7.66-7.53 (m, 1H), 7.57-7.47 (m, 6H), 7.32 (t, J=6.4 Hz, 1H), 6.85 (d, J=8.4 Hz, 2H), 6.70-6.55 (m, 1H), 6.24-6.12 (m, 1H), 5.69 (ddd, J=14.8, 8.0, 3.9 Hz, 1H), 5.41 (s, 1H), 5.09-4.80 (m, 2H), 4.26 (d, J=10.1 Hz, 2H), 4.19 (s, 2H), 4.17-4.06 (m, 1H), 4.02 (dd, J=12.0, 3.9 Hz, 1H), 3.92 (d, J=8.0 Hz, 3H), 3.78 (d, J=8.7 Hz, 5H), 3.29 (s, 2H), 3.14 (d, J=1.9 Hz, 1H), 2.98-2.92 (m, 1H), 2.87-2.68 (m, 3H), 2.62 (d, J=12.5 Hz, 3H), 2.15-1.99 (m, 4H), 1.80 (s, 1H), 1.68-1.53 (m, 2H), 1.08 (t, J=7.1 Hz, 1H), 0.98-0.88 (m, 6H), 0.82 (dd, J=23.3, 16.4 Hz, 3H), 0.74 (t, J=7.2 Hz, 3H), 0.44 (s, 2H), 0.43 (s, 3H).

### Example A118. Synthesis of (2S)—N-[(7S,13S)-21-ethyl-20-[2-(methoxymethyl)pyridin-3-yl]-17,17-dimethyl-8,14-dioxo-15-oxa-3-thia-9,21,27,28-tetraazapentacyclo [17.5.2.1^{2,5}.1^{9, 13}.0^{22,26}]octacosa-1 (25),2 (28),4, 19,22 (26), 23-hexaen-7-yl]-3-methyl-2-{N-methyl-1-[(3S)-1-(prop-2-enoyl) pyrrolidin-3-yl]formamido}butanamide

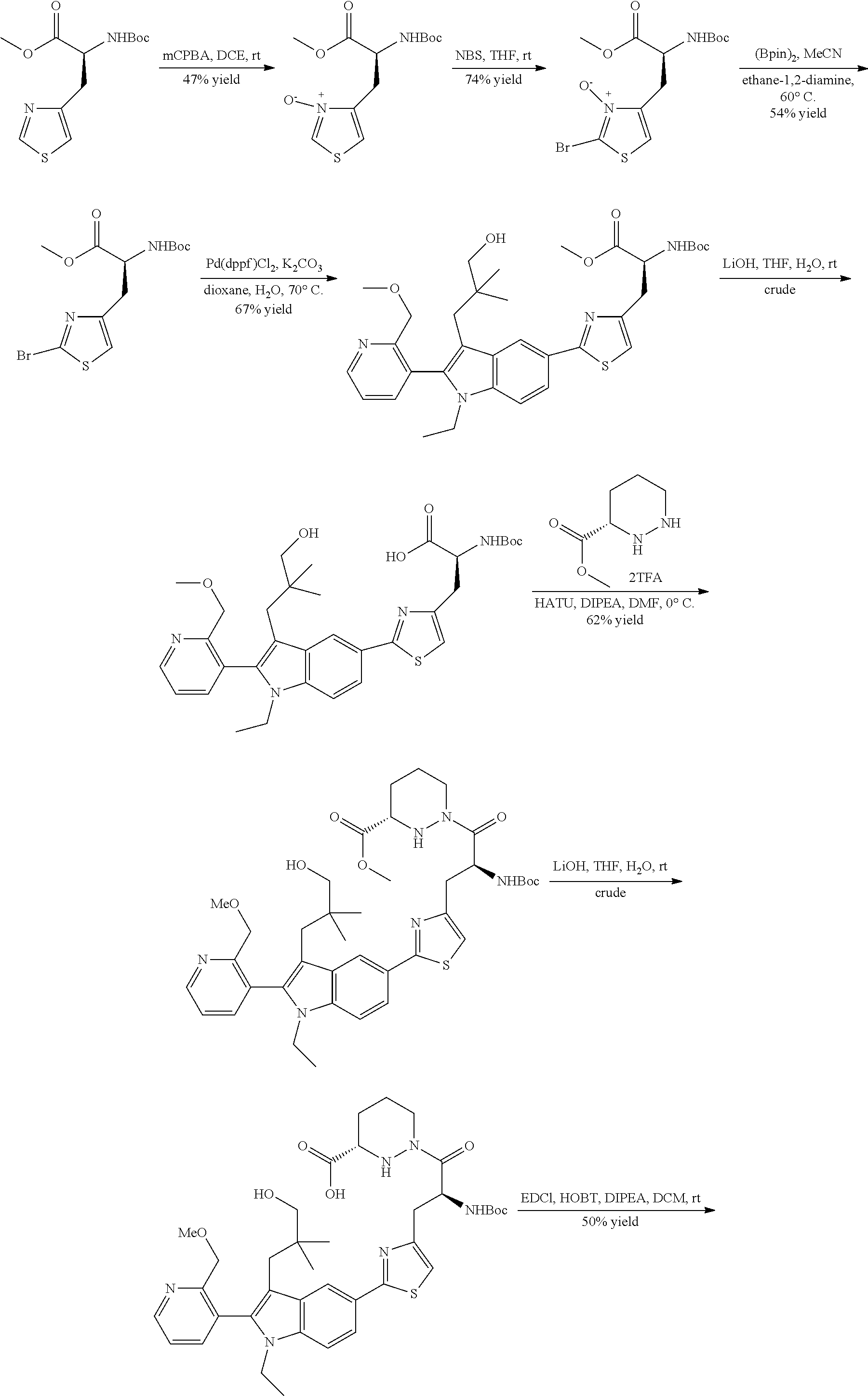

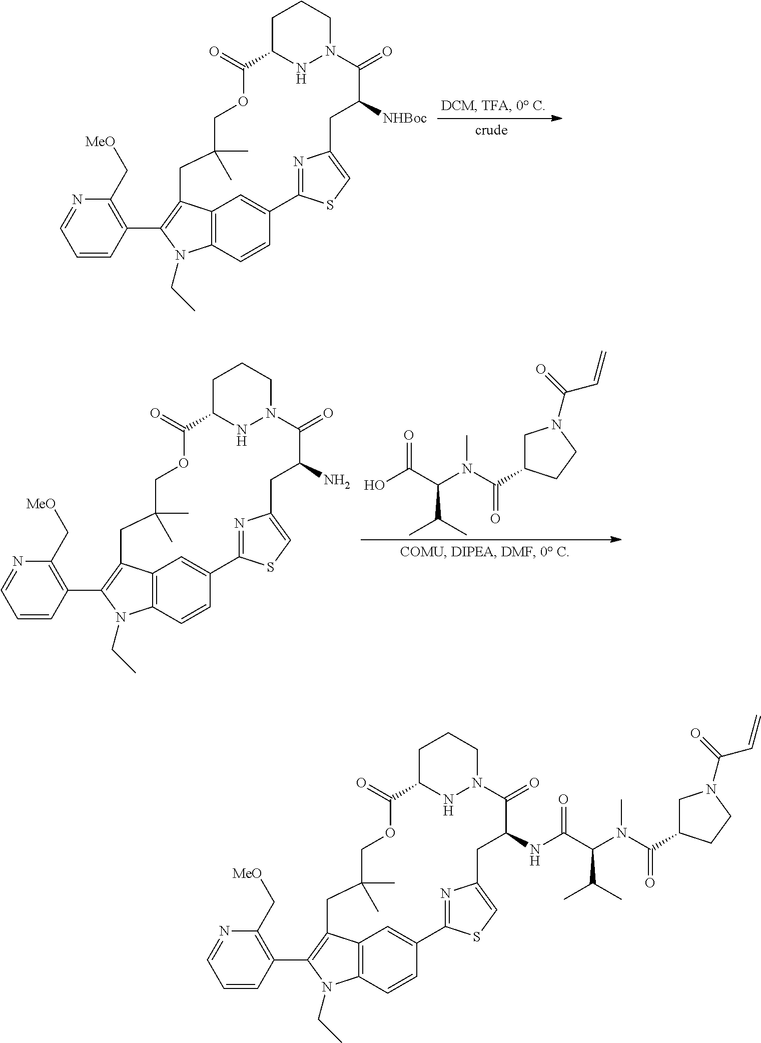 [SMILES: CCn1c(-c2cccnc2COC)c2c3cc(ccc31)-c1nc(cs1)C[C@H](NC(=O)OC(C)(C)C)C(=O)N1CCC[C@H](N1)C(=O)OCC(C)(C)C2 ; CCn1c(-c2cccnc2COC)c2c3cc(ccc31)-c1nc(cs1)C[C@H](N)C(=O)N1CCC[C@H](N1)C(=O)OCC(C)(C)C2 ; C=CC(=O)N1CC[C@H](C(=O)N(C)[C@H](C(=O)O)C(C)C)C1 ; C=CC(=O)N1CC[C@H](C(=O)N(C)[C@H](C(=O)N[C@H]2Cc3csc(n3)-c3ccc4c(c3)c(c(-c3cccnc3COC)n4CC)CC(C)(C)COC(=O)[C@@H]3CCCN(N3)C2=O)C(C)C)C1]

Step 1. A mixture of methyl (2S)-2-[(tert-butoxycarbonyl) amino]-3-(1,3-thiazol-4-yl) propanoate (2.08 g, 7.26 mmol) and mCPBA (1.88 g, 10.9 mmol) in DCE (15 mL) at 0° C. under an atmosphere of N₂ was diluted with DCM (100 mL). The mixture was allowed to warm to rt and stirred for 16 h, then diluted with DCM, washed with H₂O (1×30 mL), dried over anhydrous Na₂SO₄, and filtered. The filtrate was concentrated under reduced pressure and the residue was purified by silica gel column chromatography to give 4-[(2S)-2-[(tert-butoxycarbonyl) amino]-3-methoxy-3-oxopropyl]-1,3-thiazol-3-ium-3-olate (1.15 g, 47% yield) as a solid. LCMS (ESI): m/z [M+H] calc'd for C₁₂H₁₈N₂O₅S 302.1; found 303.2.

Step 2. To a mixture of 4-[(2S)-2-[(tert-butoxycarbonyl) amino]-3-methoxy-3-oxopropyl]-1,3-thiazol-3-ium-3-olate (1.15 g, 3.8 mmol) in THF at 0° C. under an atmosphere of N₂ was added NBS (0.74 g, 4.2 mmol) dropwise. The mixture was allowed to warm to rt and stirred for 2 h, then diluted with H₂O (500 mL) and extracted with EtOAc (3×500 mL). The combined organic layers were washed with water (2×30 mL), dried over anhydrous Na₂SO₄, and filtered. The filtrate was concentrated under reduced pressure and the residue was purified by silica gel column chromatography to give 2-bromo-4-[(2S)-2-[(tert-butoxycarbonyl) amino]-3-methoxy-3-oxopropyl]-1,3-thiazol-3-ium-3-olate (1.2 g, 74% yield) as a solid. LCMS (ESI): m/z [M+H] calc'd for C₁₂H₁₇BrN₂O₅S 380.0; found 381.0.

Step 3. To a stirred mixture of 2-bromo-4-[(2S)-2-[(tert-butoxycarbonyl) amino]-3-methoxy-3-oxopropyl]-1,3-thiazol-3-ium-3-olate (1.2 g, 3.2 mmol) and 4,4,5,5-tetramethyl-2-(tetramethyl-1,3,2-dioxaborolan-2-yl)-1,3,2-dioxaborolane (1.04 g, 4.1 mmol) in MeCN at 70° C. under an atmosphere of N₂ was added ethane-1,2-diamine (1.89 g, 31.5 mmol) in portions. The mixture was cooled to 60° C. and the mixture was stirred overnight, then diluted with water (500 mL) and extracted with EtOAc (3×400 mL). The combined organic layers were washed with brine (1×50 mL), dried over anhydrous Na₂SO₄, and filtered. The filtrate was concentrated under reduced pressure and the residue was purified by silica gel column chromatography to give methyl (2S)-3-(2-bromo-1,3-thiazol-4-yl)-2-[(tert-butoxycarbonyl) amino]propanoate (653 mg, 54% yield) as a solid.

Step 4. A 50 mL sealed tube was charged with 3-[1-ethyl-2-[2-(methoxymethyl)pyridin-3-yl]-5-(4,4,5,5-tetramethyl-1,3,2-dioxaborolan-2-yl) indol-3-yl]-2,2-dimethylpropan-1-ol (1.00 g, 2.1 mmol), K₂CO₃ (727 mg, 5.2 mmol), Pd(dppf)Cl2 (153 mg, 0.21 mmol), and 2,4-dibromo-1,3-thiazole (1.0 g, 4.2 mmol) at rt under an atmosphere of N₂, then 1,4-dioxane (1.0 mL) and H₂O (0.20 mL) were added. The mixture was heated to 70° C. and stirred for 4 h, then cooled, diluted with H₂O (100 mL) and extracted with EtOAc (3×100 mL). The combined organic layers were washed with brine (3×100 mL), dried over anhydrous Na₂SO₄, and filtered. The filtrate was concentrated under reduced pressure and the residue was purified by silica gel column chromatography to give 3-[5-(4-bromo-1,3-thiazol-2-yl)-1-ethyl-2-[2-(methoxymethyl)pyridin-3-yl]indol-3-yl]-2,2-dimethylpropan-1-ol (727 mg, 67% yield) as a solid. LCMS (ESI): m/z [M+H] calc'd for C₃₄H₄₄N₄O₆S 636.3; found 637.3.

Step 5. To a stirred mixture of methyl (2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoate (636 mg, 1.0 mmol) and LiOH·H₂O (126 mg, 3.0 mmol) in THF at 0° C. under an atmosphere of N₂ was added H₂O (1.24 mL) portionwise. The mixture was allowed to warm to rt and stirred for 1 h, then diluted with water (300 mL) and extracted with EtOAc (3×300 mL). The combined organic layers were washed with brine (1×100 mL), dried over anhydrous Na₂SO₄, filtered, and the filtrate concentrated under reduced pressure to give (2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid (622 mg, crude), which was used in the next step directly without further purification. LCMS (ESI): m/z [M+H] calc'd for C₃₃H₄₂N₄O₆S 622.3; found 623.2.

Step 6. To a stirred mixture of (2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid (622 mg, 1.0 mmol) and methyl (3S)-1,2-diazinane-3-carboxylate (288 mg, 2.0 mmol) in DMF at 0° C. under an atmosphere of N₂ was added HATU (570 mg, 1.5 mmol). The mixture was stirred at 0° C. for 1 h, then diluted with EtOAc and washed with H₂O (1×10 mL), dried over anhydrous Na₂SO₄, filtered, and the filtrate concentrated under reduced pressure to give methyl (3S)-1-[(2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate (550 mg, 62% yield) as a solid. LCMS (ESI): m/z [M+H] calc'd for C₃₉H₅₂N₆O₇S 748.4; found 749.6.

Step 7. (3S)-1-[(2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylic acid was synthesized in a manner similar to (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid except methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate was substituted with methyl (3S)-1-[(2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate. LCMS (ESI): m/z [M+H] calc'd for C₃₈H₅₀N₆O₇S 734.3; found 735.3.

Step 8. tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2 (2,4)-thiazola-1 (5,3)-indola-6 (1,3)-pyridazinacycloundecaphane-4-yl) carbamate was synthesized in a manner similar to tert-butyl ((6³S,4S)-1¹-ethyl-1²-(2-((S)-1-methoxyethyl) pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate except (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid was substituted with (3S)-1-[(2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylic acid. LCMS (ESI): m/z [M+H] calc'd for C₃₈H₄₈N₆O₆S 716.3; found 717.4.

Step 9. To a stirred mixture of tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-2 (2,4)-thiazola-1 (5,3)-indola-6 (1,3)-pyridazinacycloundecaphane-4-yl) carbamate (253 mg) in DCM at 0° C. under an atmosphere of N₂ was added TFA (1.0 mL) dropwise. The mixture was stirred at 0° C. for 1 h, then concentrated under reduced pressure and then repeated using toluene (20 mL×3) to give (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2 (2,4)-thiazola-1 (5,3)-indola-6 (1,3)-pyridazinacycloundecaphane-5,7-dione (253 mg, crude) as a solid. LCMS (ESI): m/z [M+H] calc'd for C₃₃H₄₀N₆O₄S 616.3; found 617.3.

Step 10. (2S)—N-[(7S,13S)-21-ethyl-20-[2-(methoxymethyl)pyridin-3-yl]-17,17-dimethyl-8,14-dioxo-15-oxa-3-thia-9,21,27,28-tetraazapentacyclo [17.5.2.1^{2,5}.1^{9,13}.0^{22,26}]octacosa-1 (25), 2 (28),4, 19,22 (26),23-hexaen-7-yl]-3-methyl-2-{N-methyl-1-[(3S)-1-(prop-2-enoyl) pyrrolidin-3-yl]formamido}butanamide was synthesized in a manner similar to (2S)—N-[(8S,14S)-4-amino-22-ethyl-21-[2-(2-methoxyethyl)phenyl]-18, 18-dimethyl-9, 15-dioxo-16-oxa-10,22,28-triazapentacyclo [18.5.2.1^{2,6}.110, 14.0^{23,27}]nonacosa-1 (26),2,4,6 (29),20,23 (27),24-heptaen-8-yl]-3-methyl-2-{N-methyl-1-[(3S)-1-(prop-2-enoyl) pyrrolidin-3-yl]formamido}butanamide except (6³S,4S)-4-amino-11-ethyl-25-hydroxy-1²-(2-((S)-1-methoxyethyl) pyridin-3-yl)-10,10-dimethyl-6¹,6²,6³,6⁴,6⁵,6⁶-hexahydro-11H-8-oxa-1 (5,3)-indola-6 (1,3)-pyridazina-2 (1,3)-benzenacycloundecaphane-5,7-dione was substituted with (6³S,4S,Z)-4-amino-1¹-ethyl-1²-(2-(methoxymethyl)pyridin-3-yl)-10, 10-dimethyl-6¹,6²,6³,6⁴,6⁵,6⁶-hexahydro-1¹H-8-oxa-2 (2,4)-thiazola-1 (5,3)-indola-6 (1,3)-pyridazinacycloundecaphane-5,7-dione. LCMS (ESI): m/z [M+H] calc'd for C₄₇H₆₀N₈O₇S 880.4; found 881.6; ¹H NMR (400 MHZ, DMSO-d₆) δ 8.75 (m, 1H), 8.55 (d, J=6.7 Hz, 1H), 8.32 (d, J=8.3 Hz, 1H), 7.99 (d, J=7.7 Hz, 1H), 7.65-7.51 (m, 3H), 7.11-6.92 (m, 1H), 6.72-6.56 (m, 1H), 6.18 (dd, J=16.8, 2.9 Hz, 1H), 5.82-5.65 (m, 1H), 5.61-5.46 (m, 1H), 5.02 (dd, J=24.2, 12.2 Hz, 1H), 4.69 (d, J=10.9 Hz, 1H), 4.37-4.11 (m, 5H), 4.05-3.79 (m, 4H), 3.76-3.50 (m, 6H), 3.47 (s, 2H), 3.08 (s, 3H), 3.04 (s, 1H), 2.98 (d, J=1.9 Hz, 1H), 2.95 (d, J=3.6 Hz, 2H), 2.83 (d, J=2.0 Hz, 2H), 2.24-2.03 (m, 4H), 1.81 (s, 2H), 1.56 (s, 1H), 1.11 (t, J=7.0 Hz, 2H), 1.02-0.87 (m, 8H), 0.80 (dd, J=24.6, 6.6 Hz, 3H), 0.41 (s, 2H), 0.31 (s, 1H).

### Example A194. Synthesis of (2S)—N-[(7S,13S)-21-ethyl-20-[2-(methoxymethyl)pyridin-3-yl]-17,17-dimethyl-8,14-dioxo-15-oxa-4-thia-9,21,27,28-tetraazapentacyclo [17.5.2.1^{2,5}.1^{9,13}.0^{22,26}]octacosa-1 (25),2,5 (28), 19,22 (26), 23-hexaen-7-yl]-3-methyl-2-{N-methyl-1-[(3S)-1-(prop-2-enoyl) pyrrolidin-3-yl]formamido}butanamide

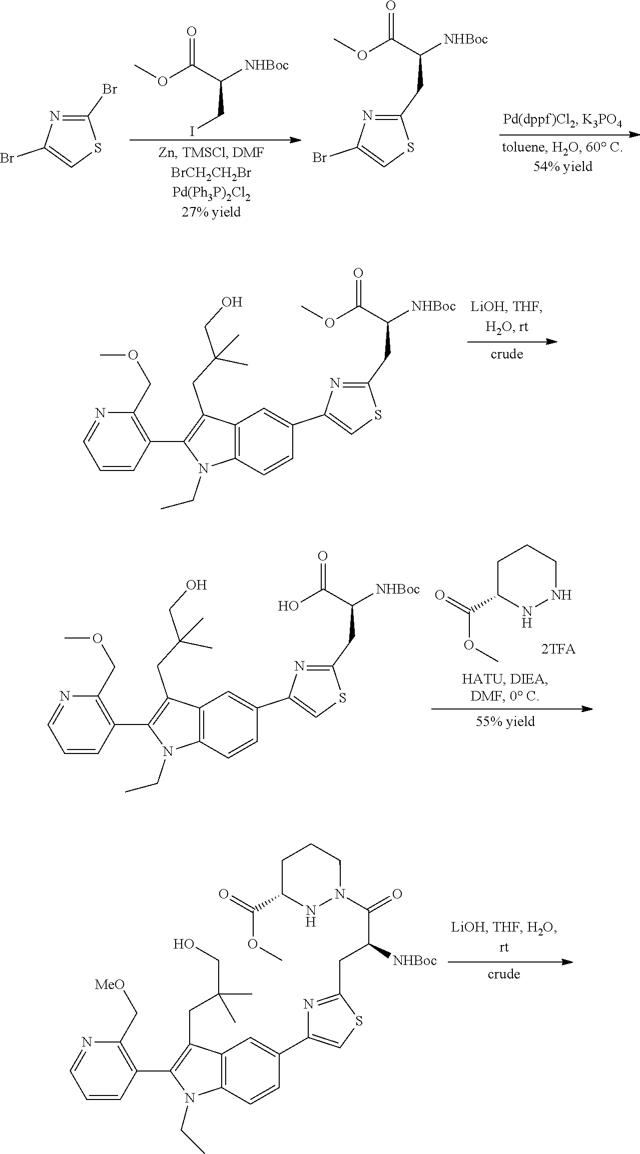

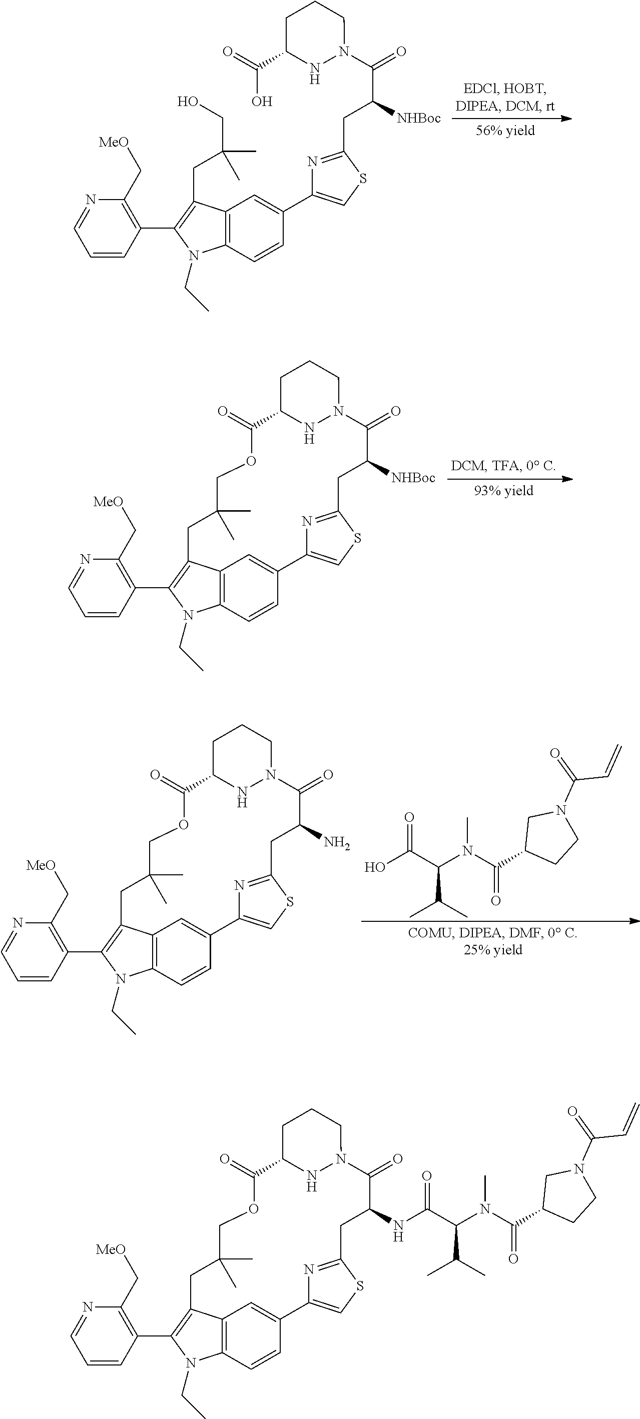 [SMILES: CCn1c(-c2cccnc2COC)c(CC(C)(C)CO)c2cc(-c3csc(C[C@H](NC(=O)OC(C)(C)C)C(=O)N4CCC[C@@H](C(=O)O)N4)n3)ccc21 ; CCn1c(-c2cccnc2COC)c2c3cc(ccc31)-c1csc(n1)C[C@H](NC(=O)OC(C)(C)C)C(=O)N1CCC[C@H](N1)C(=O)OCC(C)(C)C2 ; CCn1c(-c2cccnc2COC)c2c3cc(ccc31)-c1csc(n1)C[C@H](N)C(=O)N1CCC[C@H](N1)C(=O)OCC(C)(C)C2 ; C=CC(=O)N1CC[C@H](C(=O)N(C)[C@H](C(=O)O)C(C)C)C1 ; C=CC(=O)N1CC[C@H](C(=O)N(C)[C@H](C(=O)N[C@H]2Cc3nc(cs3)-c3ccc4c(c3)c(c(-c3cccnc3COC)n4CC)CC(C)(C)COC(=O)[C@@H]3CCCN(N3)C2=O)C(C)C)C1]

Step 1. A mixture of Zn (1.2 g, 182 mmol) and 1,2-dibromoethane (1.71 g, 9.1 mmol) and DMF (50 mL) was stirred for 30 min at 90° C. under an atmosphere of Ar. The mixture was allowed to rt, then TMSCl (198 mg, 1.8 mmol) was added dropwise over 30 min at rt. Methyl (2R)-2-[(tert-butoxycarbonyl) amino]-3-iodopropanoate (10.0 g, 30.4 mmol) in DMF (100 mL) was added dropwise over 10 min at rt. The mixture was heated to 35° C. and stirred for 2 h, then a mixture of 2,5-dibromo-1,3-thiazole (1.48 g, 60.8 mmol) and Pd(PPh₃)₂Cl₂ (2.1 g, 3.0 mmol) in DMF (100 mL) was added dropwise. The mixture was heated to 70° C. and stirred for 2 h, then filtered and the filtrate diluted with EtOAc (1 L) and washed with H₂O (3×1 L), dried with anhydrous Na₂SO₄, and filtered. The filtrate was concentrated under reduced pressure and the residue was purified by silica gel column chromatography to give methyl (2S)-3-(5-bromo-1,3-thiazol-2-yl)-2-[(tert-butoxycarbonyl) amino]propanoate (3 g, 27% yield) as a semi-solid. LCMS (ESI): m/z [M+H] calc'd for C₁₂H₁₇BrN₂O₄S 364.0; found 365.1.

Step 2. Into a 20 mL sealed tube were added 3-[1-ethyl-2-[2-(methoxymethyl)pyridin-3-yl]-5-(4,4,5,5-tetramethyl-1,3,2-dioxaborolan-2-yl) indol-3-yl]-2,2-dimethylpropan-1-ol (100 mg, 0.21 mmol), K₃PO₄ (111 mg, 0.52 mmol), Pd(dppf)Cl₂ (15 mg, 0.02 mmol), methyl (2S)-3-(4-bromo-1,3-thiazol-2-yl)-2-[(tert-butoxycarbonyl) amino]propanoate (153 mg, 0.42 mmol), toluene (1 mL), and H₂O (0.2 mL) at rt under an atmosphere of N₂. The mixture was heated to 60° C. and stirred for 3 h, cooled, diluted with H₂O (10 mL) and extracted with EtOAc (10 mL×3). The combined organic layers were washed with brine (3×10 mL), dried over anhydrous Na₂SO₄, and filtered. The filtrate was concentrated under reduced pressure and the residue was purified by silica gel column chromatography to give methyl (2S)-2-[(tert-butoxycarbonyl) amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoate (72 mg, 54% yield) as a solid. LCMS (ESI): m/z [M+H] calc'd for C₃₄H₄₄N₄O₆S 636.3; found 637.2.

Step 3. A mixture of methyl (2S)-2-[(tert-butoxycarbonyl) amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoate (40 mg, 0.06 mmol) and LiOH·H₂O (unspecified) in THF (1 mL) and H₂O (0.2 mL) was stirred at rt under an atmosphere of N₂ for 2 h. The mixture was acidified to pH 5 with aqueous NaHSO₄ and extracted with EtOAc (3×10 mL). The combined organic layers were washed with brine (3×10 mL), dried over anhydrous Na₂SO₄, filtered, and concentrated under reduced pressure to give (2S)-2-((tert-butoxycarbonyl) amino)-3-(4-(1-ethyl-3-(3-hydroxy-2,2-dimethylpropyl)-2-(2-(methoxymethyl)pyridin-3-yl)-1H-indol-5-yl) thiazol-2-yl) propanoic acid. The crude product was used in the next step directly without further purification. LCMS (ESI): m/z [M+H] calc'd for C₃₃H₄₂N₄O₆S 622.3; found 623.3.

Step 4. Methyl (3S)-1-((2S)-2-((tert-butoxycarbonyl) amino)-3-(4-(1-ethyl-3-(3-hydroxy-2,2-dimethylpropyl)-2-(2-(methoxymethyl)pyridin-3-yl)-1H-indol-5-yl) thiazol-2-yl) propanoyl) hexahydropyridazine-3-carboxylate was synthesized in a manner similar to methyl (3S)-1-[(2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoyl]-1,2-diazinane-3-carboxylate except (2S)-2-[(tert-butoxycarbonyl) amino]-3-[2-[1-ethyl-3-(3-hydroxy-2,2-dimethylpropyl)-2-[2-(methoxymethyl)pyridin-3-yl]indol-5-yl]-1,3-thiazol-4-yl]propanoic acid was substituted with (2S)-2-((tert-butoxycarbonyl) amino)-3-(4-(1-ethyl-3-(3-hydroxy-2,2-dimethylpropyl)-2-(2-(methoxymethyl)pyridin-3-yl)-1H-indol-5-yl) thiazol-2-yl) propanoic acid. LCMS (ESI): m/z [M+H] calc'd for C₃₉H₅₂N₆O₇S 748.4; found 749.4.

Step 5. (3S)-1-[(2S)-2-[(tert-butoxycarbonyl) amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoyl]-1,2-diazinane-3-carboxylic acid was synthesized in a manner similar to (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid except methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate was substituted with methyl (3S)-1-((2S)-2-((tert-butoxycarbonyl) amino)-3-(4-(1-ethyl-3-(3-hydroxy-2,2-dimethylpropyl)-2-(2-(methoxymethyl)pyridin-3-yl)-1H-indol-5-yl) thiazol-2-yl) propanoyl) hexahydropyridazine-3-carboxylate. LCMS (ESI): m/z [M+H] calc'd for C₃₈H₅₀N₆O₇S 734.3; found 735.4.

Step 6. Tert-butyl ((6³S,4S,Z)-1¹-ethyl-1²-(2-(methoxymethyl)pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate was synthesized in a manner similar to tert-butyl ((6³S,4S)-1¹-ethyl-1²-(2-((S)-1-methoxyethyl) pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate except (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid was substituted with (3S)-1-[(2S)-2-[(tert-butoxycarbonyl) amino]-3-[4-[1-ethyl-3-(3-hydroxy-2,2-dimethylpropyl)-2-[2-(methoxymethyl)pyridin-3-yl]indol-5-yl]-1,3-thiazol-2-yl]propanoyl]-1,2-diazinane-3-carboxylic acid. LCMS (ESI): m/z [M+H] calc'd for C₃₈H₄₈N₆O₆S 716.3; found 717.3.

Step 7. (6³S,4S,Z)-4-amino-1¹-ethyl-1²-(2-(methoxymethyl)pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione was synthesized in a manner similar to (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2 (2,4)-thiazola-1 (5,3)-indola-6 (1,3)-pyridazinacycloundecaphane-5,7-dione except tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-11H-8-oxa-2 (2,4)-thiazola-1 (5,3)-indola-6 (1,3)-pyridazinacycloundecaphane-4-yl) carbamate was substituted with tert-butyl ((6³S,4S,Z)-1¹-ethyl-1²-(2-(methoxymethyl)pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate. LCMS (ESI): m/z [M+Na] calc'd for C₃₃H₄₀N₆O₄SNa 639.3; found 640.3.

Step 8. (2S)—N-[(7S, 13S)-21-ethyl-20-[2-(methoxymethyl)pyridin-3-yl]-17, 17-dimethyl-8,14-dioxo-15-oxa-4-thia-9,21,27,28-tetraazapentacyclo [17.5.2.1^{2,5}.1^{9, 13}.0^{22,26}]octacosa-1 (25),2,5 (28), 19,22 (26),23-hexaen-7-yl]-3-methyl-2-{N-methyl-1-[(3S)-1-(prop-2-enoyl) pyrrolidin-3-yl]formamido}butanamide was synthesized in a manner similar to (2S)—N-[(7S,13S)-21-ethyl-20-[2-(methoxymethyl)pyridin-3-yl]-17,17-dimethyl-8, 14-dioxo-15-oxa-3-thia-9,21,27,28-tetraazapentacyclo [17.5.2.1^{2,5}.1^{9, 13}.0^{22,26}]octacosa-1 (25),2 (28),4, 19,22 (26), 23-hexaen-7-yl]-3-methyl-2-{N-methyl-1-[(3S)-1-(prop-2-enoyl) pyrrolidin-3-yl]formamido}butanamide except (6³S,4S)-4-amino-1¹-ethyl-2⁵-hydroxy-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione was substituted with (6³S,4S,Z)-4-amino-1¹-ethyl-1²-(2-(methoxymethyl)pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione. LCMS (ESI): m/z [M+H] calc'd for C₄₇H₆₀N₈O₇S 880.4; found 881.5; ¹H NMR (400 MHZ, DMSO-d₆) δ 8.70 (dt, J=16.2, 8.1 Hz, 1H), 8.54 (ddd, J=6.6, 4.7, 1.7 Hz, 1H), 8.50 (m, 1H), 7.96 (d, J=7.8 Hz, 1H), 7.88 (t, J=2.1 Hz, 2H), 6.70-6.57 (m, 2H), 6.24-6.13 (m, 2H), 5.75 (m, 1H), 5.55 (t, J=7.3 Hz, 1H), 5.46 (d, J=8.5 Hz, 1H), 5.14 (d, J=13.0 Hz, 1H), 4.84-4.75 (m, 1H), 4.35 (d, J=10.7 Hz, 1H), 4.28-4.19 (m, 4H), 3.91 (s, 3H), 3.87 (dd, J=10.4, 8.1 Hz, 1H), 3.78-3.70 (m, 2H), 3.63 (t, J=8.8 Hz, 2H), 3.61-3.49 (m, 2H), 2.87 (d, J=1.1 Hz, 2H), 2.79 (s, 1H), 2.38 (s, 1H), 2.18 (s, 1H), 2.13 (d, J=10.7 Hz, 4H), 1.96 (s, 2H), 1.81 (s, 1H), 1.53 (s, 2H), 1.11 (t, J=7.1 Hz, 2H), 0.99-0.89 (m, 7H), 0.93-0.81 (m, 2H), 0.78 (d, J=6.6 Hz, 2H), 0.28 (s, 3H).

### Example A71. Synthesis of (2S)-2-(1-{1-[(2E)-4-(dimethylamino) but-2-enoyl]azetidin-3-yl}-N-methylformamido)-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methylbutanamide

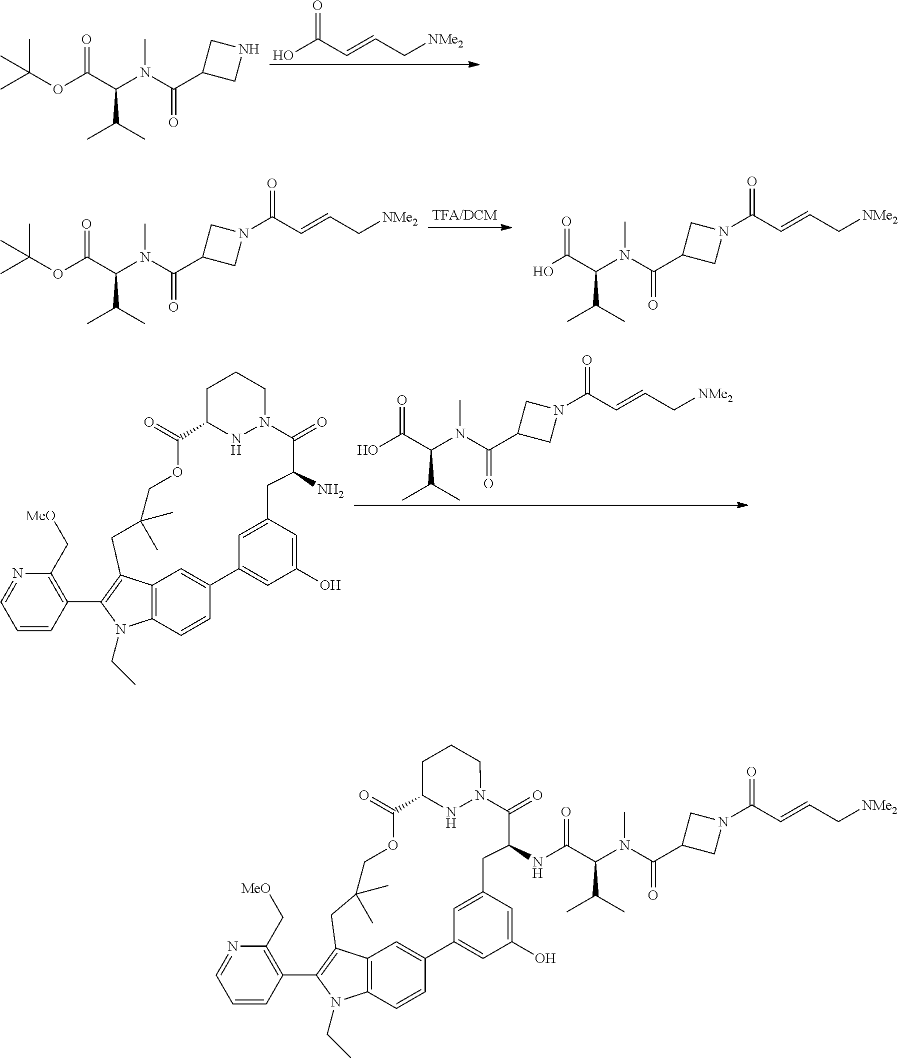

Step 1. To a mixture of tert-butyl N-(azetidine-3-carbonyl)-N-methyl-L-valinate (350 mg, 1.3 mmol) and (2E)-4-(dimethylamino) but-2-enoic acid (201 mg, 1.56 mmol) in DCM (8 mL) at 5° C. was added a solution of T3P, 50% in EtOAc (827 mg, 2.6 mmol) and DIPEA (1.7 g, 13 mmol) in DCM (2 mL). The mixture was stirred for 1 h, then diluted with EtOAc (20 mL) and H₂O (20 mL). The aqueous and organic layers were separated and the organic layer was washed with H₂O (3×10 mL), brine (10 mL), dried over anhydrous Na₂SO₄, and filtered. The filtrate was concentrated under reduced pressure and the residue was purified by prep-HPLC to give tert-butyl (E)-N-(1-(4-(dimethylamino) but-2-enoyl) azetidine-3-carbonyl)-N-methyl-L-valinate (200 mg, 39% yield) as a solid. LCMS (ESI): m/z [M+H] calc'd for C₂₀H₃₅N₃O₄ 381.3; found 382.3.

Step 2. To a mixture of tert-butyl (E)-N-(1-(4-(dimethylamino) but-2-enoyl) azetidine-3-carbonyl)-N-methyl-L-valinate (190 mg, 0.32 mmol) in DCM (3 mL) at rt was added TFA (1 mL).

The mixture was stirred at rt for 1 h, then concentrated under reduced pressure to give (E)-N-(1-(4-(dimethylamino) but-2-enoyl) azetidine-3-carbonyl)-N-methyl-L-valine (190 mg, 90%) as a solid, which was used directly in the next step without further purification. LCMS (ESI): m/z [M+H] calc'd for C₁₆H₂₇N₃O₄ 325.2; found 326.2.

Step 3. To a mixture of (6³S,4S)-4-amino-1¹-ethyl-25-hydroxy-1²-(2-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (172 mg, 0.27 mmol) and (E)-N-(1-(4-(dimethylamino) but-2-enoyl) azetidine-3-carbonyl)-N-methyl-L-valine (105 mg, 0.32 mmol) in DMF (2 mL) at 5° C. was added a mixture of HATU (133 mg, 0.297 mmol) and DIPEA (348 mg, 2.7 mmol) in DMF (1 mL). The mixture was stirred for 1 h, then diluted with EtOAc (20 mL) and H₂O (20 mL). The aqueous and organic layers were separated, and the organic layer was washed with H₂O (3×10 mL), brine (10 mL), dried over anhydrous Na₂SO₄, and filtered. The filtrate was concentrated under reduced pressure and the residue was purified by prep-TLC to give (2S)-2-(1-{1-[(2E)-4-(dimethylamino) but-2-enoyl]azetidin-3-yl}-N-methylformamido)-N-[(8S, 14S)-22-ethyl-4-hydroxy-21-[2-(methoxymethyl)pyridin-3-yl]-18, 18-dimethyl-9, 15-dioxo-16-oxa-10,22,28-triazapentacyclo [18.5.2.1^{2,6}.1^{10, 14}.0^{23,27}]nonacosa-1 (26),2,4,6 (29),20,23 (27),24-heptaen-8-yl]-3-methylbutanamide (4.8 mg, 2% yield over 2 steps) as a solid. LCMS (ESI): m/z [M+H] calc'd for C₅₂H₆₈N₈O₈ 932.5; found 933.5; ¹H NMR (400 MHZ, CD₃OD) δ 8.71 (d, J=3.2 Hz, 1H), 8.50 (s, 1.5H), 8.08-7.85 (m, 2H), 7.65-7.44 (m, 3H), 7.32-7.14 (m, 1H), 7.07-6.95 (m, 1H), 6.80 (dt, J =22.1, 6.8 Hz, 1H), 6.55 (d, J=35.8 Hz, 1H), 6.30 (d, J=15.4 Hz, 1H), 5.56 (dd, J=13.8, 6.7 Hz, 1H), 4.76 (dd, J=19.8, 10.5 Hz, 1H), 4.54 (dd, J=15.9, 7.5 Hz, 2H), 4.48-4.38 (m, 2H), 4.36-4.23 (m, 3H), 4.22-4.14 (m, 1H), 3.96 (qd, J=15.6, 7.9 Hz, 3H), 3.77 (ddd, J=25.8, 23.4, 11.9 Hz, 2H), 3.58 (dd, J=17.2, 8.3 Hz, 2H), 3.38 (s, 2H), 3.25-3.11 (m, 3H), 3.05-2.94 (m, 1H), 2.94-2.81 (m, 4H), 2.73 (dd, J=20.9, 11.0 Hz, 1H), 2.45 (d, J=6.9 Hz, 5H), 2.32-2.07 (m, 3H), 1.92 (d, J=13.2 Hz, 1H), 1.72 (s, 1H), 1.64-1.51 (m, 1H), 1.18 (t, J=7.0 Hz, 2H), 1.00 (ddd, J=14.6, 11.8, 8.5 Hz, 6H), 0.92-0.81 (m, 4H), 0.55-0.41 (m, 3H).

### Example A67. Synthesis of (2E)-4-(dimethylamino)-N-(6-{[(1S)-1-{[(8S,14S)-22-ethyl-4-hydroxy-21-[2-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl](methyl) carbamoyl}pyridin-3-yl) but-2-enamide

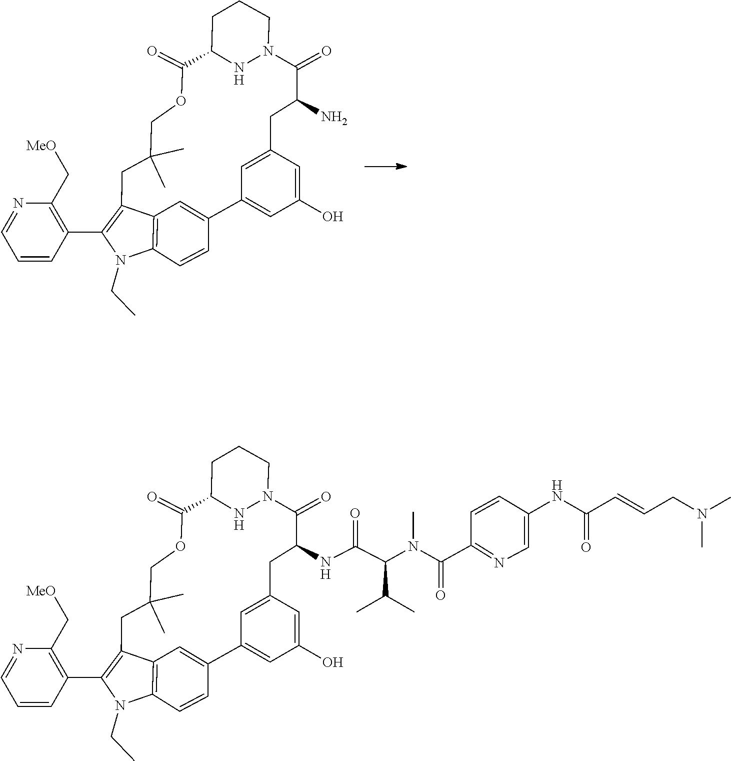 [SMILES: CCn1c(-c2cccnc2COC)c2c3cc(ccc31)-c1cc(O)cc(c1)C[C@H](N)C(=O)N1CCC[C@H](N1)C(=O)OCC(C)(C)C2 ; CCn1c(-c2cccnc2COC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2]

Step 1. To a mixture of (6³S,4S)-4-amino-1¹-ethyl-2⁵-hydroxy-1²-(2-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione TFA salt (225 mg, 0.28 mmol) and (E)-N-(5-(4-(dimethylamino) but-2-enamido) picolinoyl)-N-methyl-L-valine TFA salt (260 mg crude, 0.56 mmol) in DMF (5 mL) at 0° C. were added DIPEA (0.46 mL, 2.8 mmol) followed by HATU (140 mg, 0.36 mmol). The mixture was stirred at 0-10° C. for 1 h, then concentrated under reduced pressure and the residue was purified by prep-HPLC to give (2E)-4-(dimethylamino)-N-(6-{[(1S)-1-{[(8S,14S)-22-ethyl-4-hydroxy-21-[2-(methoxymethyl)pyridin-3-yl]-18, 18-dimethyl-9, 15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]carbamoyl}-2-methylpropyl](methyl) carbamoyl}pyridin-3-yl) but-2-enamide TFA salt (23.3 mg, 8% yield over 2 steps) as a solid. LCMS (ESI): m/z [M+Na] calc'd for C₅₄H₆₇N₉O₈Na 992.5; found 992.4; ¹H NMR (400 MHZ, CD₃OD) δ 9.05 (d, J=2.5 Hz, 1H), 8.85-8.71 (m, 1H), 8.43 (ddd, J=33.3, 18.0, 2.6 Hz, 2H), 8.01-7.87 (m, 2H), 7.83-7.70 (m, 1H), 7.60-7.47 (m, 2H), 7.31-7.19 (m, 1H), 7.07-6.90 (m, 2H), 6.70-6.36 (m, 3H), 5.81-5.61 (m, 1H), 4.50-4.20 (m, 4H), 4.01-3.68 (m, 3H), 3.64-3.35 (m, 5H), 3.27-3.08 (m, 3H), 3.04-2.44 (m, 11H), 2.36-2.10 (m, 3H), 1.93 (d, J=13.0 Hz, 1H), 1.61 (dd, J=34.3, 21.6 Hz, 3H), 1.39-1.16 (m, 3H), 1.12-0.81 (m, 6H), 0.78-0.45 (m, 6H).

### Example A54. Synthesis of (2S)-2-{1-[(3S)-1-[(2E)-4-(dimethylamino) but-2-enoyl]pyrrolidin-3-yl]-N-methylformamido}-N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-3-methylbutanamide

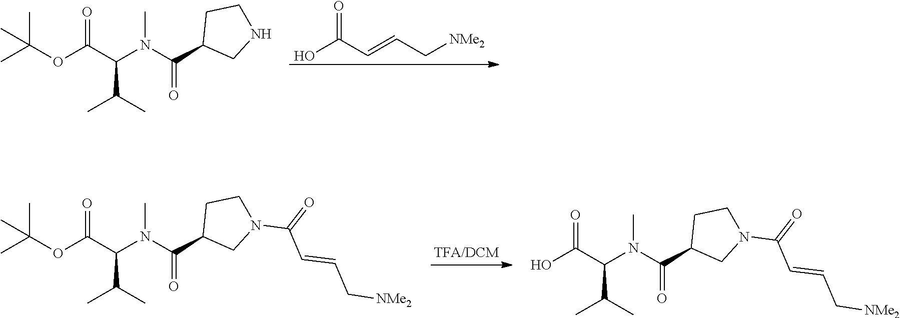 [SMILES: CC(C)[C@@H](C(=O)OC(C)(C)C)N(C)C(=O)[C@H]1CCNC1 ; CN(C)C/C=C/C(=O)O ; CC(C)[C@@H](C(=O)OC(C)(C)C)N(C)C(=O)[C@H]1CCN(C(=O)/C=C/CN(C)C)C1 ; CC(C)[C@@H](C(=O)O)N(C)C(=O)[C@H]1CCN(C(=O)/C=C/CN(C)C)C1]

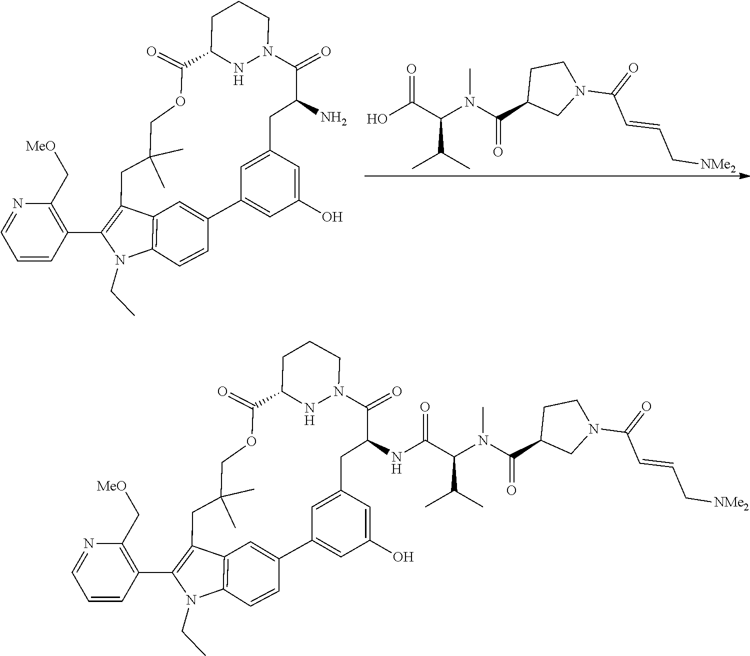 [SMILES: CCn1c(-c2cccnc2COC)c2c3cc(ccc31)-c1cc(O)cc(c1)C[C@H](N)C(=O)N1CCC[C@H](N1)C(=O)OCC(C)(C)C2 ; CC(C)[C@@H](C(=O)O)N(C)C(=O)[C@H]1CCN(C(=O)/C=C/CN(C)C)C1 ; CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)/C=C/CN(C)C)C1)C(=O)N1CCC[C@H](N1)C(=O)OCC(C)(C)C2]

Step 1. To a mixture of tert-butyl N-methyl-N—((S)-pyrrolidine-3-carbonyl)-L-valinate (210 mg, 0.73 mmol) in DMF (4 mL) at rt were added 4-(dimethylamino)-4-methylpent-2-ynoic acid (450 mg, 2.9 mmol), DIPEA (1.2 mL, 7.3 mmol), and HATU (332 mg, 0.88 mmol). The mixture was stirred at rt for 1 h then diluted with EtOAc, and the mixture washed with H₂O, brine, dried over Na₂SO₄, and filtered. The filtrate was concentrated under reduced pressure and the residue was purified by silica gel column chromatography to give tert-butyl N—((S)-1-(4-(dimethylamino)-4-methylpent-2-ynoyl) pyrrolidine-3-carbonyl)-N-methyl-L-valinate (140 mg, 45% yield) as an oil. LCMS (ESI): m/z [M+H] calc'd for C₂₃H₃₉N₃O₄ 421.3; found 422.3.

Step 2. A mixture of tert-butyl N—((S)-1-(4-(dimethylamino)-4-methylpent-2-ynoyl) pyrrolidine-3-carbonyl)-N-methyl-L-valinate (130 mg, 0.31 mmol) in DCM (2 mL) and TFA (1 mL) was stirred at rt for 90 min. The mixture was concentrated under reduced pressure to give N—((S)-1-(4-(dimethylamino)-4-methylpent-2-ynoyl) pyrrolidine-3-carbonyl)-N-methyl-L-valine TFA salt (150 mg) as an oil, which was used directly in the next step without further purification. LCMS (ESI): m/z [M+H] calc'd for C₁₉H₃₁N₃O₄ 365.2; found 366.2.

Step 3. (3S)-1-(4-(dimethylamino)-4-methylpent-2-ynoyl)-N-((2S)-1-(((6³S,4S)-1¹-ethyl-25-hydroxy-1²-(2-(methoxymethyl)pyridin-3-yl)-10, 10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide TFA salt was synthesized in a manner similar to 1-acryloyl-N-((2S)-1-(((6³S,4S)-1¹-ethyl-25-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylazetidine-3-carboxamide except (2S)-2-{1-[(3S)-1-[(2E)-4-(dimethylamino) but-2-enoyl]pyrrolidin-3-yl]-N-methylformamido}-N-[(8S, 14S)-22-ethyl-4-hydroxy-21-[2-(methoxymethyl)pyridin-3-yl]-18, 18-dimethyl-9, 15-dioxo-16-oxa-10,22,28-triazapentacyclo [18.5.2.1^{2,6}. 1^{10,1}4.0^{23,27}]nonacosa-1 (26),2,4,6 (29),20,23 (27),24-heptaen-8-yl]-3-methylbutanamide TFA salt. (120 mg, 54% yield over 2 steps) as a solid. 1H-NMR (400 MHZ, CD₃OD) δ 8.76-8.68 (m, 1H), 8.44 (s, 1H), 8.02-7.94 (m, 1H), 7.94-7.84 (m, 1H), 7.65-7.43 (m, 3H), 7.27-7.14 (m, 1H), 7.06-6.96 (m, 1H), 6.65-6.48 (m, 1H), 5.62-5.46 (m, 1H), 4.81-4.57 (m, 1H), 4.46-4.22 (m, 3H), 4.10-3.35 (m, 11H), 3.26-2.93 (m, 6H), 2.91-2.51 (m, 4H), 2.42-2.09 (m, 9H), 1.95-1.87 (m, 1H), 1.85-1.40 (m, 6H), 1.38 -1.10 (m, 6H), 1.07-0.81 (m, 9H), 0.56-0.38 (m, 3H). LCMS (ESI): m/z [M+H] C₅₂H₆₈N₈O₈ found 947.7.

### Example A95. Synthesis of (2S)—N-[(8S,14S)-22-ethyl-4-hydroxy-21-[2-(methoxymethyl)pyridin-3-yl]-18,18-dimethyl-9,15-dioxo-16-oxa-10,22,28-triazapentacyclo [18.5.2.1^{2,6}.1^{10,14}.0^{23,27}]nonacosa-1 (26), 2,4,6 (29), 20,23 (27),24-heptaen-8-yl]-3-methyl-2-{N-methyl-1-[(3S)-1-[4-(morpholin-4-yl) but-2-ynoyl]pyrrolidin-3-yl]formamido}butanamide

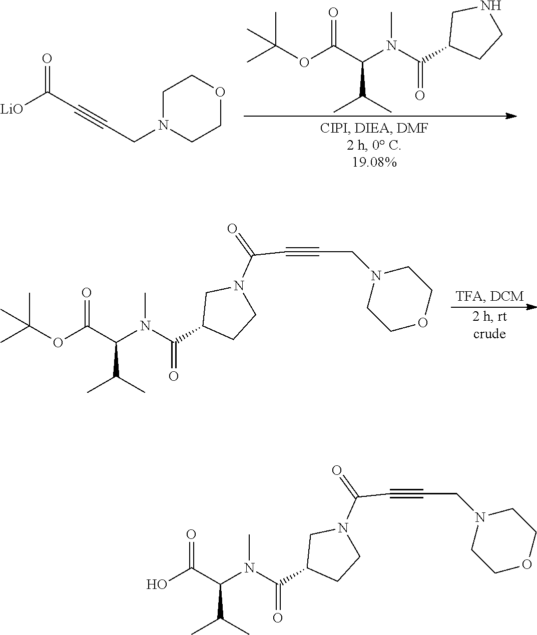

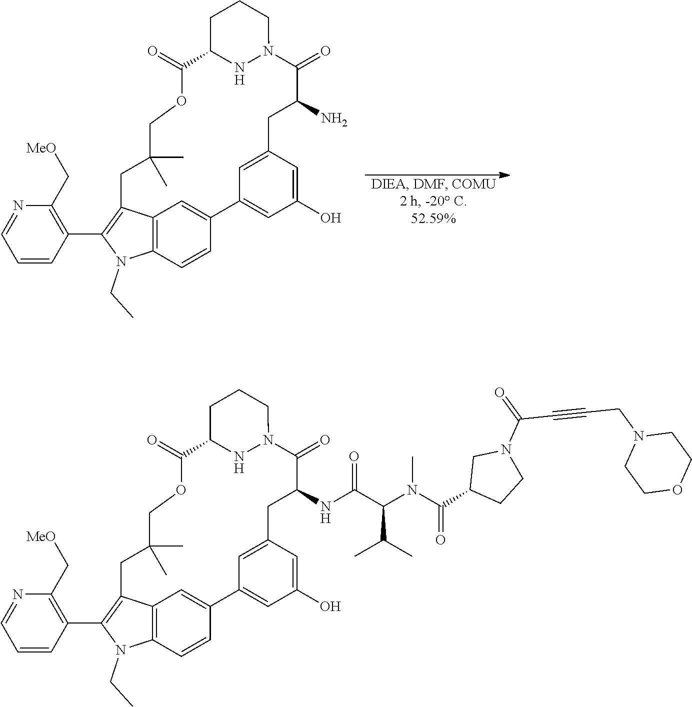 [SMILES: CCn1c(-c2cccnc2COC)c2c3cc(ccc31)-c1cc(O)cc(c1)C[C@H](N)C(=O)N1CCC[C@H](N1)C(=O)OCC(C)(C)C2 ; "DIEA, DMF, COMU2 h, -20° C.52.59%" ; CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2]

Step 1. A mixture of tert-butyl (2S)-3-methyl-2-[N-methyl-1-(3S)-pyrrolidin-3-ylformamido]butanoate (500 mg, 1.8 mmol), 4-(morpholin-4-yl) but-2-ynoic acid (1.49 g, 8.8 mmol), DIPEA (682 mg, 5.3 mmol) and CIP (635 mg, 2.3 mmol) in DMF (5 mL) was stirred at 0° C. for 2 h. The mixture was concentrated under reduced pressure and the residue was purified by silica gel column chromatography to give tert-butyl N-methyl-N—((S)-1-(4-morpholinobut-2-ynoyl) pyrrolidine-3-carbonyl)-L-valinate (150 mg, 19% yield) as an oil. LCMS (ESI): m/z [M+H] calc'd for C₂₃H₃₇N₃O₅ 435.3; found 436.5.

Step 2. A mixture of tert-butyl N-methyl-N—((S)-1-(4-morpholinobut-2-ynoyl) pyrrolidine-3-carbonyl)-L-valinate (250 mg, 0.57 mmol) in DCM (5 mL) and TFA (2.5 mL) was stirred at rt for 2 h. The mixture was concentrated under reduced pressure to give (2S)-3-methyl-2-[N-methyl-1-[(3S)-1-[4-(morpholin-4-yl) but-2-ynoyl]pyrrolidin-3-yl]formamido]butanoic acid (310 mg, crude) as an oil, which was used directly in the next step without further purification. LCMS (ESI): m/z [M+H] calc'd for C₁₉H₂₉N₃O₅ 379.2; found 380.2.

Step 3. A mixture of (6³S,4S)-4-amino-1¹-ethyl-25-hydroxy-1²-(2-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (250 mg, 0.4 mmol), DIPEA (516 mg, 4.0 mmol), (2S)-3-methyl-2-[N-methyl-1-[(3S)-1-[4-(morpholin-4-yl) but-2-ynoyl]pyrrolidin-3-yl]formamido]butanoic acid (182 mg, 0.48 mmol), and COMU (205 mg, 0.48 mmol) in DMF (3 mL) was stirred at −20° C. for 2 h. The mixture was diluted with H₂O (10 mL), then extracted with EtOAc (3×10 mL) and the combined organic layers were washed with brine (3×10 mL), dried over anhydrous Na₂SO₄, and filtered. The mixture was concentrated under reduced pressure and the residue was purified by reverse-phase silica gel column chromatography to give (2S)—N-[(8S, 14S)-22-ethyl-4-hydroxy-21-[2-(methoxymethyl)pyridin-3-yl]-18, 18-dimethyl-9, 15-dioxo-16-oxa-10,22,28-triazapentacyclo [18.5.2.1^{2,6}.1^{10,1}4.0^{23,27}]nonacosa-1 (26),2,4,6 (29),20,23 (27),24-heptaen-8-yl]-3-methyl-2-{N-methyl-1-[(3S)-1-[4-(morpholin-4-yl) but-2-ynoyl]pyrrolidin-3-yl]formamido}butanamide (207 mg, 53% yield) as a solid. LCMS (ESI): m/z [M+H] calc'd for C₅₅H₇₀N₈O₉ 986.5; found 987.8; 1H NMR (400 MHZ, DMSO-d₆) δ 9.39-9.28 (m, 1H), 8.74 (t, J=4.8, 1H), 8.70-8.04 (m, 1H), 7.98-7.90 (m, 1.5H), 7.82 (d, J=7.7 Hz, 0.5H), 7.63-7.46 (m, 3H), 7.26-7.10 (m, 1H), 7.03 (s, 1H), 6.58-6.43 (m, 1H), 5.44-5.30 (m, 1H), 5.06 (q, 0.5H), 4.72 (t, J=11.0, 0.5H), 4.39-4.20 (m, 3H), 4.15 (d, J=11.1 Hz, 1H), 4.09-3.85 (m, 4H), 3.66 (s, 2H), 3.65-3.58 (m, 4H), 3.58-3.55 (m, 2H), 3.55-3.48 (m, 3H), 3.47-3.41 (m, 3H), 3.31 (s, 2H), 3.10 (s, 2H), 2.92 (s, 1H), 2.89-2.65 (m, 5H), 2.68 (s, 1H), 2.45-2.38 (m, 1H), 2.29-2.24 (m, 1H), 2.23-1.99 (m, 3H), 1.82 (d, J=12.1 Hz, 1H), 1.76-1.62 (m, 1H), 1.61-1.45 (m, 1H), 1.14-1.04 (m, 2H), 1.02-0.92 (m, 3H), 0.91-0.86 (m, 3H), 0.83-0.77 (m, 3H), 0.77-0.70 (m, 2H), 0.50-0.35 (m, 3H).

### Example A145. Synthesis of two atropisomers of (2S)-2-{1-[(3S)-1-(but-2-ynoyl) pyrrolidin-3-yl]-N-methylformamido}-N-[(8S,14S,20M)-22-ethyl-4-hydroxy-21-{2-[(1S)-1-methoxyethyl]pyridin-3-yl}-18, 18-dimethyl-9,15-dioxo-16-oxa-10,22,28-triazapentacyclo [18.5.2.1^{2,6}.1^{10,14}.0^{23,27}]nonacosa-1 (26),2,4,6 (29),20,23 (27),24-heptaen-8-yl]-3-methylbutanamide

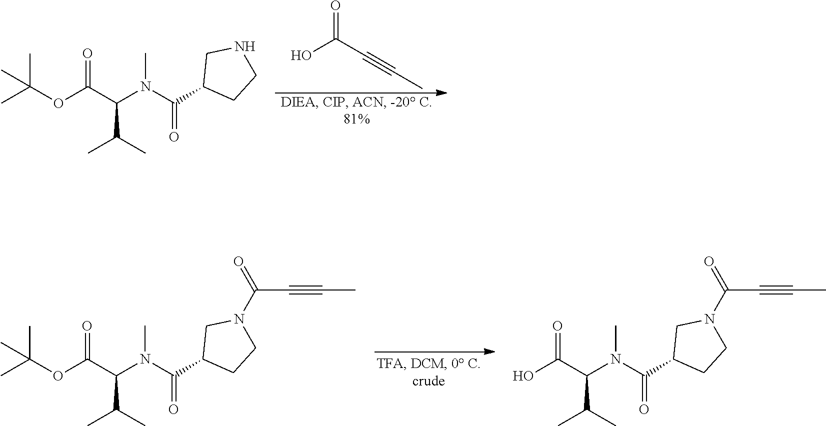

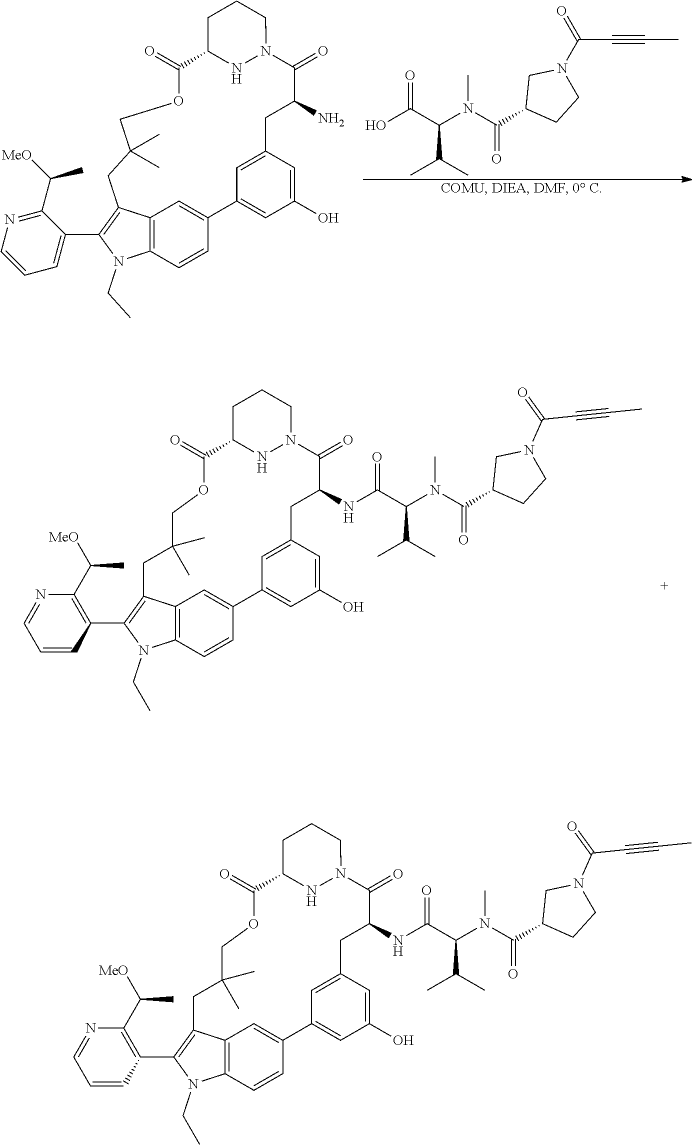

Step 1. To a mixture of but-2-ynoic acid (222 mg) and CIP (588 mg) in ACN (8 mL) at 0° C. under an atmosphere of Ar was added DIPEA (681 mg). The mixture was stirred at 0° C. then tert-butyl N-methyl-N—((S)-pyrrolidine-3-carbonyl)-L-valinate (500 mg) in ACN (3 mL) was added dropwise and the mixture stirred at 0° C. for 2 h. EtOAc was added and the mixture was washed with brine (3×20 mL), dried over anhydrous Na₂SO₄, and filtered. The filtrate was concentrated under reduced pressure and the residue was purified by silica gel column chromatography to give tert-butyl N—((S)-1-(but-2-ynoyl) pyrrolidine-3-carbonyl)-N-methyl-L-valinate as a solid. LCMS (ESI): m/z [M+H] calc'd for C₁₉H₃₀N₂O₄ 350.2; found 352.1.

Step 2. A mixture of tert-butyl N—((S)-1-(but-2-ynoyl) pyrrolidine-3-carbonyl)-N-methyl-L-valinate (200 mg) in DCM (4 mL) and TFA (2 mL) was stirred at 0° C. for 2 h. The mixture was concentrated under reduced pressure with azeotropic removal of H₂O using toluene (4 mL×2) to give N—((S)-1-(but-2-ynoyl) pyrrolidine-3-carbonyl)-N-methyl-L-valinate as a solid. LCMS (ESI): m/z [M+H] calc'd for C₁₅H₂₂N₂O₄ 294.2; found 295.2.

Step 3. Two atropisomers of (2S)-2-{1-[(3S)-1-(but-2-ynoyl) pyrrolidin-3-yl]-N-methylformamido}-N-[(8S, 14S,20M)-22-ethyl-4-hydroxy-21-{2-[(1S)-1-methoxyethyl]pyridin-3-yl}-18,18-dimethyl-9,15-dioxo-16-oxa-10,22,28-triazapentacyclo [18.5.2.1^{2,6}.1^{10, 14}.0^{23,27}]nonacosa-1 (26),2,4,6 (29),20,23 (27),24-heptaen-8-yl]-3-methylbutanamide was synthesized in a manner similar to (2S)—N-[(8S,14S)-22-ethyl-4-hydroxy-21-[2-(methoxymethyl)pyridin-3-yl]-18, 18-dimethyl-9,15-dioxo-16-oxa-10,22,28-triazapentacyclo [18.5.2.1^{2,6}. 1^{10,14}.0^{23,27}]nonacosa-1 (26),2,4,6 (29),20,23 (27),24-heptaen-8-yl]-3-methyl-2-{N-methyl-1-[(3S)-1-[4-(morpholin-4-yl) but-2-ynoyl]pyrrolidin-3-yl]formamido}butanamide except (2S)-3-methyl-2-[N-methyl-1-[(3S)-1-[4-(morpholin-4-yl) but-2-ynoyl]pyrrolidin-3-yl]formamido]butanoic acid was substituted with N—((S)-1-(but-2-ynoyl) pyrrolidine-3-carbonyl)-N-methyl-L-valinate. (43.3 mg, 12% yield) and (33 mg, 9% yield) both as solids. LCMS (ESI): m/z [M+H] calc'd for C₅₂H₆₅N₇O₈ 915.5; found 916.7; ¹H NMR (400 MHZ, DMSO-d₆) δ 9.34-9.27 (m, 1H), 8.78 (t, J=2.5 Hz, 1H), 8.68 (t, J=8.5 Hz, 0.5H), 8.20-8.11 (m, 0.6H), 7.95 (ddt, J=5.4, 3.5, 1.7 Hz, 2H), 7.63-7.60 (m, 1H), 7.61-7.49 (m, 2H), 7.13 (s, 1H), 7.03 (d, J=6.2 Hz, 1H), 6.60-6.49 (d, J=35.5 Hz, 1H), 5.43-5.39 (m, 1H), 5.12-5.00 (m, 0.7H), 4.74 (d, J=10.6 Hz, 0.4H), 4.32-4.25 (m, 1H), 4.18-3.85 (m, 5H), 3.81-3.45 (m, 8H), 3.18-3.02 (m, 5H), 2.93-2.80 (m, 4H), 2.80-2.70 (m, 2H), 2.42-2.36 (m, 1H), 2.31-2.20 (m, 1H), 2.18-1.96 (m, 6H), 1.85-1.74 (m, 1H), 1.74-1.63 (m, 1H), 1.62-1.42 (m, 1H), 1.32-1.16 (m, 4H), 1.15-1.05 (t, J=6.3 Hz, 4H), 1.04-0.95 (m, 2H), 0.95-0.85 (m, 5H), 0.68-0.52 (m, 4H), 0.52-0.37 (m, 4H). and LCMS (ESI): m/z [M+H] calc'd for C₅₂H₆₅N₇O₈ 915.5; found 916.7; ¹H NMR (400 MHZ, DMSO-d₆) δ 9.36-9.28 (m, 1H), 8.77 (dd, J=4.7, 1.8 Hz, 1H), 8.62-8.57 (m, 0.5H), 8.15-8.07 (m, 0.5H), 7.95 (s, 1H), 7.87-7.81 (m, 1H), 7.65-7.51 (m, 3H), 7.37-7.25 (m, 1H), 7.10-7.03 (m, 1H), 6.54 (d, J=35.5 Hz, 1H), 5.52-5.21 (m, 2H), 4.78-4.66 (m, 0.5H), 4.34-4.20 (m, 3H), 4.15-3.85 (m, 4H), 3.85-3.42 (m, 7H), 3.22-3.11 (m, 3H), 2.97-2.72 (m, 7H), 2.62-2.54 (m, 1H), 2.28-1.96 (m, 7H), 1.95-1.74 (m, 2H), 1.73-1.44 (m, 2H), 1.42-1.37 (m, 3H), 1.28-1.14 (m, 1H), 1.03-0.85 (m, 6H), 0.83-0.72 (m, 7H), 0.71-0.55 (m, 3H).

### Example A28. Synthesis of (2S)—N-[(8S,14S)-22-ethyl-4-hydroxy-21-[4-(methoxymethyl)pyridin-3-yl]-18,18-dimethyl-9,15-dioxo-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]-3-methyl-2-[N-methyl-1-[(3S)-1-(prop-2-enoyl) pyrrolidin-3-yl]formamido]butanamide

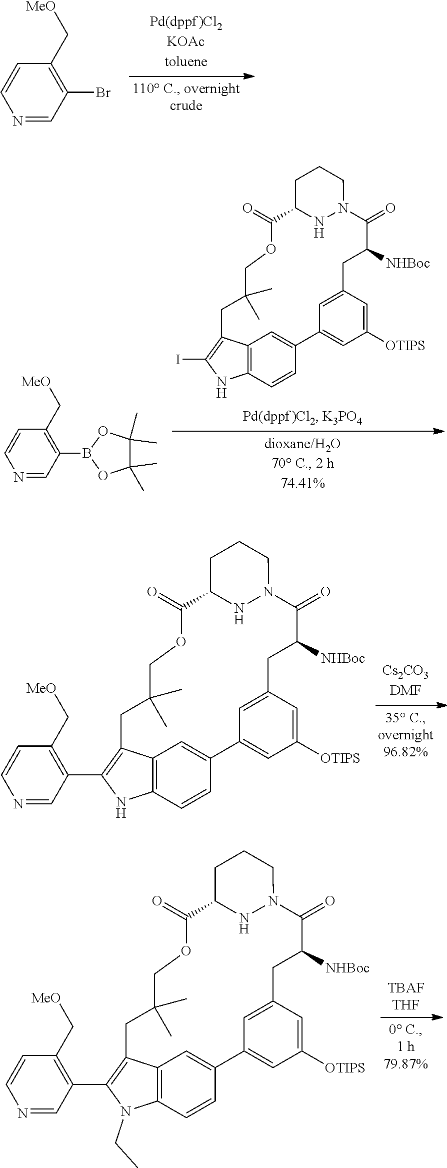

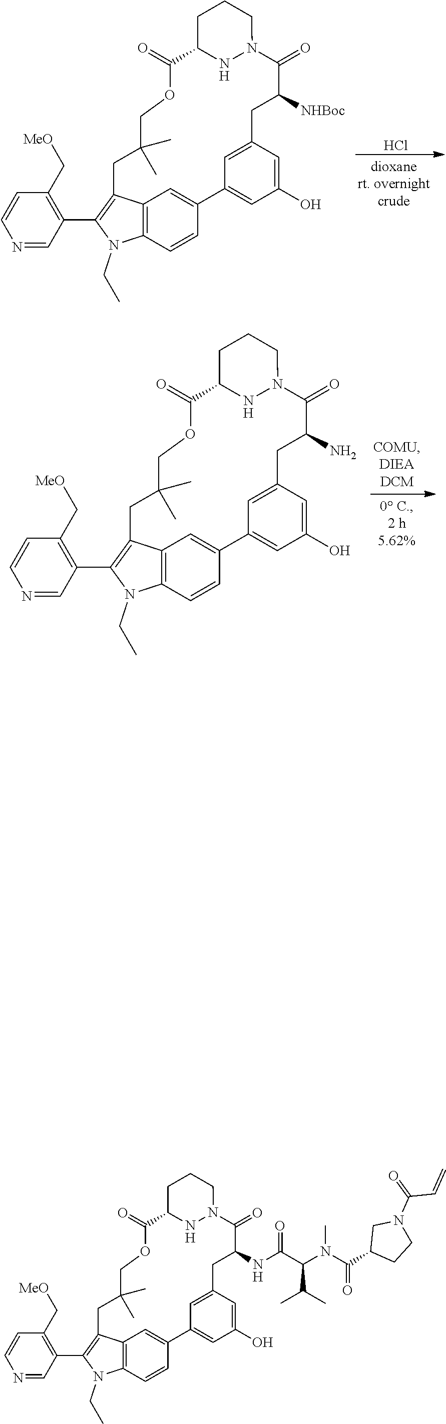 [SMILES: CCn1c(-c2cnccc2COC)c2c3cc(ccc31)-c1cc(O)cc(c1)C[C@H](NC(=O)OC(C)(C)C)C(=O)N1CCC[C@H](N1)C(=O)OCC(C)(C)C2 ; Cl ; CCn1c(-c2cnccc2COC)c2c3cc(ccc31)-c1cc(O)cc(c1)C[C@H](N)C(=O)N1CCC[C@H](N1)C(=O)OCC(C)(C)C2 ; C=CC(=O)N1CC[C@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)C1]

Step 1. To a mixture of 3-bromo-4-(methoxymethyl)pyridine (1.00 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at rt under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl) pyridine, which was used directly in the next step directly without further purification. LCMS (ESI): m/z [M+H] calc'd for C₁₃H₂₀BNO₃ 249.2; found 250.3.

Step 2. To a mixture of 4-(methoxymethyl)-3-(4,4,5,5-tetramethyl-1,3,2-dioxaborolan-2-yl) pyridine (290 mg, 1.16 mmol), K₃PO₄ (371 mg, 1.75 mmol) and tert-butyl N-[(8S,14S)-21-iodo-18,18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaen-8-yl]carbamate (500 mg, 0.58 mmol) in 1,4-dioxane (5 mL) and H₂O (1 mL) at rt under an atmosphere of Ar was added Pd(dppf)Cl₂ (43 mg, 0.06 mmol). The mixture was heated to 70° C. and stirred for 2 h, then H₂O added and the mixture extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography to give tert-butyl N-[(8S, 14S)-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]. 1\hat{ }[10, 14]0.0\hat{ }[23,27]nonacosa-1 (26),2,4,6 (29),20,23 (27),24-heptaen-8-yl]carbamate (370 mg, 74% yield) as a foam. LCMS (ESI): m/z [M+H] calc'd for C₄₈H₆₇N₅O₇Si 853.6; found 854.6.

Step 3. A mixture of tert-butyl N-[(8S,14S)-21-[4-(methoxymethyl)pyridin-3-yl]-18,18-dimethyl-9, 15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]nonacosa-1 (26),2,4,6 (29),20,23 (27),24-heptaen-8-yl]carbamate (350 mg, 0.41 mmol), Cs₂CO₃ (267 mg, 0.82 mmol) and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄, and filtered. The filtrate was concentrated under reduced pressure and the residue was purified by silica gel column chromatography to give tert-butyl N-[(8S, 14S)-22-ethyl-21-[4-(methoxymethyl)pyridin-3-yl]-18, 18-dimethyl-9, 15-dioxo-4-[(triisopropylsilyl)oxy]-16-oxa-10,22,28-triazapentacyclo[18.5.2.1\hat{ }[2,6]0.1\hat{ }[10, 14]0.0\hat{ }[23,27]]nonacosa-1 (26),2,4,6 (29),20,23 (27),24-heptaen-8-yl]carbamate (350 mg, 97% yield) as an oil. LCMS (ESI): m/z [M+H] calc'd for C₅₀H₇₁N₅O₇Si 881.5; found 882.6.

Step 4. A mixture of tert-butyl N-[(8S, 14S)-22-ethyl-21-[4-(methoxymethyl)pyridin-3-yl]-18,18-dimethyl-9,15-dioxo-4-[(triisopropylsilyl)oxy]-16-oxa-10,22,28-triazapentacyclo[18.5.2.1\hat{ }[2,6]0.1\hat{ }[10,14]0.0\hat{ }[23,27]nonacosa-1 (26),2,4,6 (29),20,23 (27),24-heptaen-8-yl]carbamate (350 mg, 0.4 mmol) and 1M TBAF in THF (0.48 mL, 0.480 mmol) in THF (3 mL) at 0° C. under an atmosphere of Ar was stirred for 1 h. The mixture was concentrated under reduced pressure and the residue was purified by silica gel column chromatography to give tert-butyl N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo[18.5.2.1\hat{ }[2,6]0.1\hat{ }[10, 14]0.0\hat{ }[23,27]]nonacosa-1 (26),2,4,6 (29),20,23 (27),24-heptaen-8-yl]carbamate (230 mg, 80% yield) as an oil. LCMS (ESI): m/z [M+H] calc'd for C₄₁H₅₁N₅O₇ 725.4; found 726.6.

Step 5. To a mixture of tert-butyl N-[(8S, 14S)-22-ethyl-4-hydroxy-21-[4-(methoxymethyl)pyridin-3-yl]-18, 18-dimethyl-9, 15-dioxo-16-oxa-10,22,28-triazapentacyclo[18.5.2.1\hat{ }[2,6]. 1\hat{ }[10, 14]0.0\hat{ }[23,27]nonacosa-1 (26),2,4,6 (29),20,23 (27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to rt and was stirred overnight, then concentrated under reduced pressure to give (8S,14S)-8-amino-22-ethyl-4-hydroxy-21-[4-(methoxymethyl)pyridin-3-yl]-18, 18-dimethyl-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaene-9,15-dione (200 mg). LCMS (ESI): m/z [M+H] calc'd for C₃₆H₄₃N₅O₅ 625.3; found 626.5.

Step 6. To a mixture of (2S)-3-methyl-2-[N-methyl-1-[(3S)-1-(prop-2-enoyl) pyrrolidin-3-yl]formamido]butanoic acid (108 mg, 0.38 mmol) and (8S,14S)-8-amino-22-ethyl-4-hydroxy-21-[4-(methoxymethyl)pyridin-3-yl]-18, 18-dimethyl-16-oxa-10,22,28-triazapentacyclo[18.5.2.1\hat{ }[2,6]0.1\hat{ }[10, 14]0.0\hat{ }[23,27]nonacosa-1 (26),2,4,6 (29),20,23 (27),24-heptaene-9,15-dione (200 mg, 0.32 mmol) in DCM (3 mL) at 0° C. was added DIPEA (165 mg, 1.3 mmol) and COMU (274 mg, 0.64 mmol) in portions. The mixture was stirred at 0° C. for 2 h, H₂O added and extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography then prep-HPLC to give (2S)—N-[(8S,14S)-22-ethyl-4-hydroxy-21-[4-(methoxymethyl)pyridin-3-yl]-18, 18-dimethyl-9, 15-dioxo-16-oxa-10,22,28-triazapentacyclo[18.5.2.1\hat{ }[2,6]0.1\hat{ }[10,14]0.0\hat{ }[23,27]]nonacosa-1 (26),2,4,6 (29),20,23 (27),24-heptaen-8-yl]-3-methyl-2-[N-methyl-1-[(3S)-1-(prop-2-enoyl) pyrrolidin-3-yl]formamido]butanamide (16 mg, 5.6% yield) as a solid. LCMS (ESI): m/z [M+H] calc'd for C₅₀H₆₃N₇O₈ 889.5; found 890.6; ¹H NMR (400 MHZ, DMSO-d₆) δ 9.33 (dd, J=9.1, 6.9 Hz, 1H), 8.79-8.46 (m, 2H), 7.93 (s, 1H), 7.68-7.58 (m, 2H), 7.53 (t, J=8.5 Hz, 1H), 7.26-6.98 (m, 2H), 6.71-6.47 (m, 2H), 6.24-6.07 (m, 1H), 5.80-5.60 (m, 1H), 5.49-5.18 (m, 1H), 4.45-4.07 (m, 4H), 4.08-3.87 (m, 3H), 3.87-3.64 (m, 4H), 3.64-3.40 (m, 5H), 3.34 (s, 2H), 3.30 (s, 2H), 3.23 (d, J=1.8 Hz, 1H), 2.94-2.74 (m, 6H), 2.16-2.01 (m, 3H), 1.82-1.47 (m, 3H), 1.08 (q, J=8.9, 8.0 Hz, 1H), 1.00-0.88 (m, 6H), 0.82 (d, J=10.8 Hz, 4H), 0.76-0.66 (m, 2H), 0.44 (d, J=14.2 Hz, 3H).

### Example A316. Synthesis of (2R)-2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

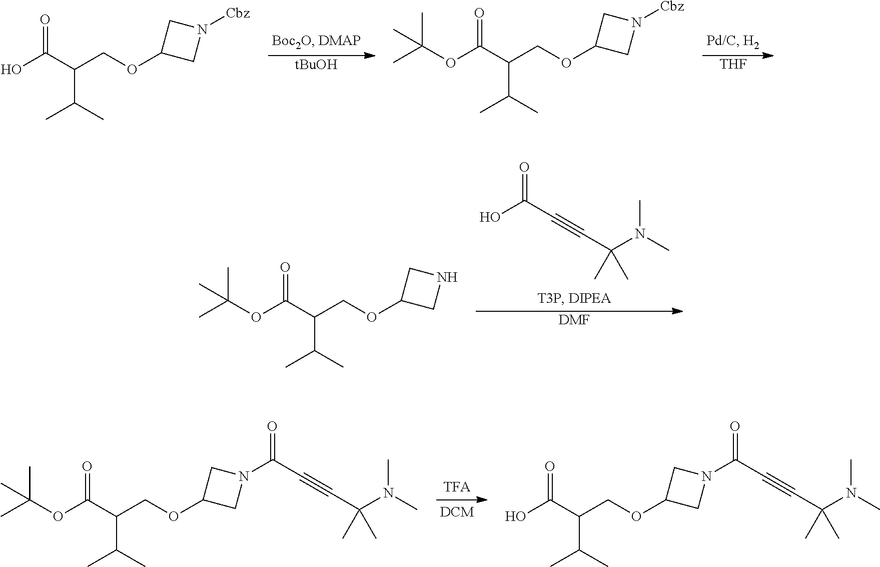

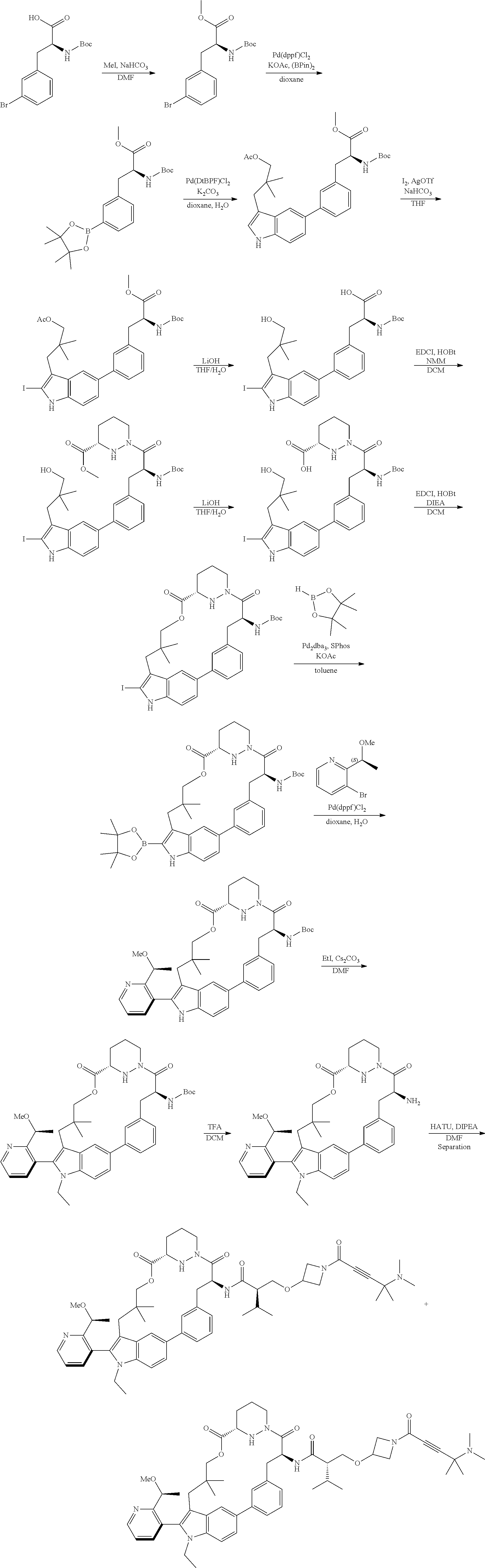

Step 1. To a mixture of 2-(((1-((benzyloxy) carbonyl) azetidin-3-yl)oxy) methyl)-3-methylbutanoic acid (650 mg, 2 mmol) and di-tert-butyl dicarbonate (883 mg, 4 mmol) in ^{t}BuOH (10 mL) was added 4-dimethylaminopyridine (124 mg, 1 mmol). The mixture was heated to 30° C. and stirred for 1 h, then diluted with H₂O (50 mL) and extracted with EtOAc (50 mL×3). The combined organic layers were concentrated under reduced pressure and the residue was purified by silica gel column chromatography to afford benzyl 3-(2-(tert-butoxycarbonyl)-3-methylbutoxy) azetidine-1-carboxylate (450 mg, 56% yield) as an oil. LCMS (ESI): m/z [M+Na] calc'd for C₂₁H₃₁NO₅Na 400.2; found 400.2.

Step 2. A mixture of benzyl 3-(2-(tert-butoxycarbonyl)-3-methylbutoxy) azetidine-1-carboxylate (450 mg, 1.19 mmol) and Pd/C (50 mg) in THF (30 mL) was stirred for 2 h under an atmosphere of H₂ (15 psi). The mixture was filtered and the filtrate was concentrated under reduced pressure to give tert-butyl 2-((azetidin-3-yloxy) methyl)-3-methylbutanoate (300 mg, 100% yield) as an oil. LCMS (ESI): m/z [M+H] calc'd for C₁₃H₂₆NO₃ 243.2; found 244.2; ¹H NMR (400 MHZ, CDCl₃) δ 4.35-4.25 (m, 1H), 3.71-3.63 (m, 2H), 3.63-3.56 (m, 2H), 3.50 (t, J=8.0 Hz, 1H), 3.43 (dd, J=9.0, 4.0 Hz, 1H), 2.37-2.26 (m, 1H), 2.21 (br. s, 1H), 1.92-1.81 (m, 1H), 1.47 (s, 9H), 0.93 (d, J=6.8 Hz, 6H).

Step 3. To a mixture of tert-butyl 2-((azetidin-3-yloxy) methyl)-3-methylbutanoate (270 mg, 1.11 mmol), 4-(dimethylamino)-4-methylpent-2-ynoic acid (860 mg, 5.55 mmol) and DIPEA (1.56 g, 11.1 mmol) in DMF (20 mL) at 0° C. was added T3P (2.12 g, 6.7 mmol). The mixture was stirred at 0° C. for 1 h, diluted with EtOAc (200 mL), then washed with H₂O (30 mL×5), brine (30 mL), dried over anhydrous Na₂SO₄ and filtered. The filtrate was concentrated under reduced pressure and the residue was purified by silica gel column chromatography to give tert-butyl 2-(((1-(4-(dimethylamino)-4-methylpent-2-ynoyl) azetidin-3-yl)oxy) methyl)-3-methylbutanoate (200 mg, 47% yield) as an oil. LCMS (ESI): m/z [M+H] calc'd for C₂₁H₃₆N₂O₄ 380.3; found 381.3.

Step 4. To a mixture of tert-butyl 2-(((1-(4-(dimethylamino)-4-methylpent-2-ynoyl) azetidin-3-yl)oxy) methyl)-3-methylbutanoate (190 mg, 0.5 mmol) in DCM (4 mL) was added TFA (2 mL). The mixture was stirred for 1 h, then concentrated under reduced pressure to give 2-(((1-(4-(dimethylamino)-4-methylpent-2-ynoyl) azetidin-3-yl)oxy) methyl)-3-methylbutanoic acid (162 mg, 100% yield) as an oil, which was used directly in the next step without further purification. LCMS (ESI): m/z [M+H] calc'd for C₁₇H₂₈N₂O₄ 324.2; found 325.3.

Step 5. To a solution of (2S)-3-(3-bromophenyl)-2-[(tert-butoxycarbonyl) amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel chromatography (13% EtOAc/pet. ether) to give methyl(S)-3-(3-bromophenyl)-2-((tert-butoxycarbonyl) amino) propanoate (109 g, crude). LCMS (ESI): m/z [M+Na] calc'd for C₁₅H₂₀BrNO₄ 380.05; found 380.0.

Step 6. To a stirred solution of methyl (2S)-3-(3-bromophenyl)-2-[(tert-butoxycarbonyl) amino]propanoate (108 g, 301.5 mmol) and bis (pinacolato)diboron (99.53 g, 391.93 mmol) in 1,4-dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel chromatography (5% EtOAc/pet. ether) to give methyl(S)-2-((tert-butoxycarbonyl) amino)-3-(3-(4,4,5,5-tetramethyl-1,3,2-dioxaborolan-2-yl)phenyl) propanoate (96 g, 78.6% yield). LCMS (ESI): m/z [M+Na] calc'd for C₂₁H₃₂BNO₆ 428.22; found 428.1.

Step 7. To a mixture of methyl (2S)-2-[(tert-butoxycarbonyl) amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in 1,4-dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel chromatography (20% EtOAc/pet. ether) to give methyl(S)-3-(3-(3-(3-acetoxy-2,2-dimethylpropyl)-1H-indol-5-yl)phenyl)-2-((tert-butoxycarbonyl) amino) propanoate (130 g, crude). LCMS (ESI): m/z [M+H] calc'd for C₃₀H₃₈N₂O₆ 523.28; found 523.1.

Step 8. To a solution of methyl (2S)-3-(3-[3-[3-(acetyloxy)-2,2-dimethylpropyl]-1H-indol-5-yl]phenyl)-2-[(tert-butoxycarbonyl) amino]propanoate (95.0 g, 181.8 mmol) and iodine (36.91 g, 145.41 mmol) in THF (1 L) at −10° C. was added AgOTf (70.0 g, 272.7 mmol) and NaHCO₃ (22.9 g, 272.65 mmol). The reaction mixture was stirred for 30 min and was then quenched by the addition of sat. Na₂S203 (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel chromatography (50% EtOAc/pet. ether) to give methyl(S)-3-(3-(3-(3-acetoxy-2,2-dimethylpropyl)-2-iodo-1H-indol-5-yl)phenyl)-2-((tert-butoxycarbonyl) amino) propanoate (49.3 g, 41.8% yield). LCMS (ESI) m/z: [M+H] calcd for C₃₀H₃₇IN₂O₆: 649.18; found 649.1.

Step 9. To a solution of methyl (2S)-3-(3-[3-[3-(acetyloxy)-2,2-dimethylpropyl]-2-iodo-1H-indol-5-yl]phenyl)-2-[(tert-butoxycarbonyl) amino]propanoate (60 g, 92.5 mmol) in THF (600 mL) was added a solution of LiOH H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with sat. brine (2×500 mL), dried over Na₂SO₄, filtered, and concentrated under reduced pressure to give (S)-2-((tert-butoxycarbonyl) amino)-3-(3-(3-(3-hydroxy-2,2-dimethylpropyl)-2-iodo-1H-indol-5-yl)phenyl) propanoic acid (45 g, 82.1% yield). LCMS (ESI): m/z [M+Na] calc'd for C₂₇H₃₃IN₂O₆ 615.13; found 615.1.

Step 10. To a solution of (2S)-2-[(tert-butoxycarbonyl) amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBT (2.05 g, 15.19 mmol), and EDCI (19.41 g, 101.27 mmol). The reaction mixture was stirred overnight and then the mixture was washed with sat. NH₄Cl (2×200 mL) and sat. brine (2×200 mL), and the mixture was dried over Na₂SO₄, filtered, and concentrated under reduced pressure to give methyl(S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylate (14 g, 38.5% yield). LCMS (ESI): m/z [M+H] calc'd for C₃₃H₄₃IN₄O₆ 718.23; found 719.4.

Step 11. To a solution of methyl(S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylate (92 g, 128.0 mmol) in THF (920 mL) at 0° C. was added a solution of LiOH H₂O (26.86 g, 640.10 mmol) in H₂O (640 mL). The reaction mixture was stirred for 2 h and was then concentrated under reduced pressure to give (S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylic acid (90 g, crude). LCMS (ESI): m/z [M+H] calc'd for C₃₂H₄₁IN₄O₆ 705.22; found 705.1.

Step 12. To a solution of (3S)-1-[(2S)-2-[(tert-butoxycarbonyl) amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel chromatography (50% EtOAc/pet. ether) to give tert-butyl ((6³S,4S)-1²-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (70 g, 79.8% yield). LCMS (ESI): m/z [M+H] calc'd for C₃₂H₃₉IN₄O₅ 687.21; found 687.1.

Step 13. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (22.0 g, 32.042 mmol), toluene (300.0 mL), Pd₂(dba)₃ (3.52 g, 3.845 mmol), S-Phos (3.95 g, 9.613 mmol), and KOAc (9.43 g, 96.127 mmol) at room temperature. To the mixture was added 4,4,5,5-tetramethyl-1,3,2-dioxaborolane (26.66 g, 208.275 mmol) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford tert-butyl ((6³S,4S)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (22 g, 90% yield) as a solid. LCMS (ESI): m/z [M+H] calc'd for C₃₈H₅₁BN₄O₇ 687.3; found 687.4.

Step 14. A mixture of tert-butyl ((6³S,4S)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (2.0 g, 2.8 mmol), 3-bromo-2-[(1S)-1-methoxyethyl]pyridine (0.60 g, 2.8 mmol), Pd(dppf)Cl₂ (0.39 g, 0.5 mmol), and K₃PO₄ (1.2 g, 6.0 mmol) in 1,4-dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to give tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.5 g, 74% yield) as a solid. LCMS (ESI): m/z [M+H] calc'd for C₄₀H₄₉N₅O₆ 695.4; found 696.5.

Step 15. To a solution of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (20 g, 28.7 mmol) and Cs₂CO₃ (18.7 g, 57.5 mmol) in DMF (150 mL) at 0° C. was added a solution of ethyl iodide (13.45 g, 86.22 mmol) in DMF (50 mL). The resulting mixture was stirred overnight at 35° C. and was then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel chromatography to give tert-butyl ((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (4.23 g, 18.8% yield) and the atropisomer (5.78 g, 25.7% yield) as solids. LCMS (ESI): m/z [M+H] calc'd for C₄₂H₅₃N₅O₆ 724.4; found 724.6.

Step 16. A mixture of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.3 g, 1.7 mmol) in TFA (10 mL) and DCM (20 mL) was stirred at 0° C. for 2 h. The mixture was concentrated under reduced pressure to afford (6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (1.30 g, crude) as a solid. LCMS (ESI): m/z [M+H] calc'd for C₃₇H₄₅N₅O₄ 623.3; found 624.4.

Step 17. To a mixture of (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (258 mg, 0.41 mmol) and 2-(((1-(4-(dimethylamino)-4-methylpent-2-ynoyl) azetidin-3-yl)oxy) methyl)-3-methylbutanoic acid (162 mg, 0.5 mmol) in DMF (4 mL) at 0° C. was added a mixture of HATU (188 mg, 0.5 mmol) and DIPEA (534 mg, 4.14 mmol) in DMF (2 mL). The mixture was stirred at 0° C. for 1 h, then diluted with H₂O (30 mL) and extracted with EtOAc (30 mL×3). The combined organic layers were concentrated under reduced pressure and the residue was purified by silica gel column chromatography to give 2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1H-8-oxa-1 (5,3)-indola-6 (1,3)-pyridazina-2 (1,3)-benzenacycloundecaphane-4-yl)-3-methylbutanamide (250 mg, 64% yield) as a solid. LCMS (ESI): m/z [M+H] calc'd for C₅₄H₇₁N₇O₇ 929.5; found 930.5; ¹H NMR (400 MHZ, CD₃OD) δ 8.90-8.79 (m, 1H), 8.54-8.21 (m, 1H), 8.15-7.91 (m, 2H), 7.88-7.67 (m, 2H), 7.65-7.52 (m, 2H), 7.47-7.15 (m, 2H), 5.80-5.52 (m, 1H), 4.53-4.23 (m, 5H), 4.23-3.93 (m, 3H), 3.90-3.76 (m, 2H), 3.75-3.58 (m, 3H), 3.57-3.44 (m, 1H), 3.38 (s, 1H), 3.29-3.26 (m, 2H), 3.21-2.85 (m, 8H), 2.82-2.65 (m, 3H), 2.51-2.30 (m, 1H), 2.24-2.03 (m, 1H), 1.99-1.87 (m, 1H), 1.86-1.69 (m, 6H), 1.67-1.57 (m, 2H), 1.57-1.39 (m, 4H), 1.45-1.05 (m, 2H), 1.04-0.96 (m, 3H), 0.96-0.88 (m, 3H), 0.88-0.79 (m, 3H), 0.79-0.63 (m, 3H), 0.56 (s, 1H).

Step 18. 2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1H-8-oxa-1 (5,3)-indola-6 (1,3)-pyridazina-2 (1,3)-benzenacycloundecaphane-4-yl)-3-methylbutanamide (180 mg, 0.194 mmol) was purified by prep-HPLC to afford (2R)-2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (41.8 mg, 23.2% yield) as a solid. LCMS (ESI): m/z [M+H] calc'd for C₅₄H₇₁N₇O₇ 930.5; found 930.5; ¹H NMR (400 MHZ, MeOD) δ 8.74 (d, J=4.0 Hz, 1H), 8.53-8.30 (m, 1H), 8.10-7.95 (m, 1H), 7.94-7.80 (m, 2H), 7.68 (t, J=8.0 Hz, 1H), 7.65-7.58 (m, 1H), 7.58-7.46 (m, 2H), 7.38-7.17 (m, 2H), 5.73-5.60 (m, 1H), 4.52-4.40 (m, 1H), 4.35-4.15 (m, 4H), 4.14-3.95 (m, 2H), 3.90-3.72 (m, 3H), 3.71-3.45 (m, 4H), 3.30-3.20 (m, 3H), 3.06-2.72 (m, 5H), 2.49-2.28 (m, 4H), 2.28-2.20 (m, 3H), 2.18-2.06 (m, 1H), 2.00-1.90 (m, 1H), 1.90-1.52 (m, 4H), 1.52-1.40 (m, 5H), 1.40-1.22 (m, 4H), 1.09-0.92 (m, 8H), 0.90-0.75 (m, 3H), 0.71-0.52 (m, 3H) and (2S)-2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (51.2 mg, 28.4% yield) as a solid. LCMS (ESI): m/z [M+H] calc'd for C₅₄H₇₁N₇O₇ 930.5; found 930.3; ¹H NMR (400 MHz, MeOD) δ 8.74 (d, J=4.0 Hz, 1H), 8.28-8.20 (m, 0.6H), 8.11-7.98 (m, 1H), 7.97-7.80 (m, 2H), 7.73-7.48 (m, 4H), 7.46-7.36 (m, 0.4H), 7.33-7.26 (m, 1H), 7.25-7.13 (m, 1H), 5.79-5.66 (m, 1H), 4.54-4.43 (m, 1H), 4.42-4.01 (m, 7H), 3.90-3.75 (m, 2H), 3.73-3.48 (m, 4H), 3.27-3.12 (m, 3H), 3.08-2.99 (m, 1H), 2.96-2.85 (m, 2H), 2.84-2.69 (m, 2H), 2.69-2.49 (m, 6H), 2.41-2.29 (m, 1H), 2.15-2.05 (m, 1H), 1.95-1.85 (m, 1H), 1.84-1.71 (m, 1H), 1.71-1.38 (m, 11H), 1.14-1.00 (m, 3H), 1.00-0.71 (m, 9H), 0.70-0.56 (m, 3H).

### Example A427. Synthesis of 3-((1-(4-(dimethylamino)-4-methylpent-2-ynoyl) azetidin-3-yl)oxy)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) propanamide

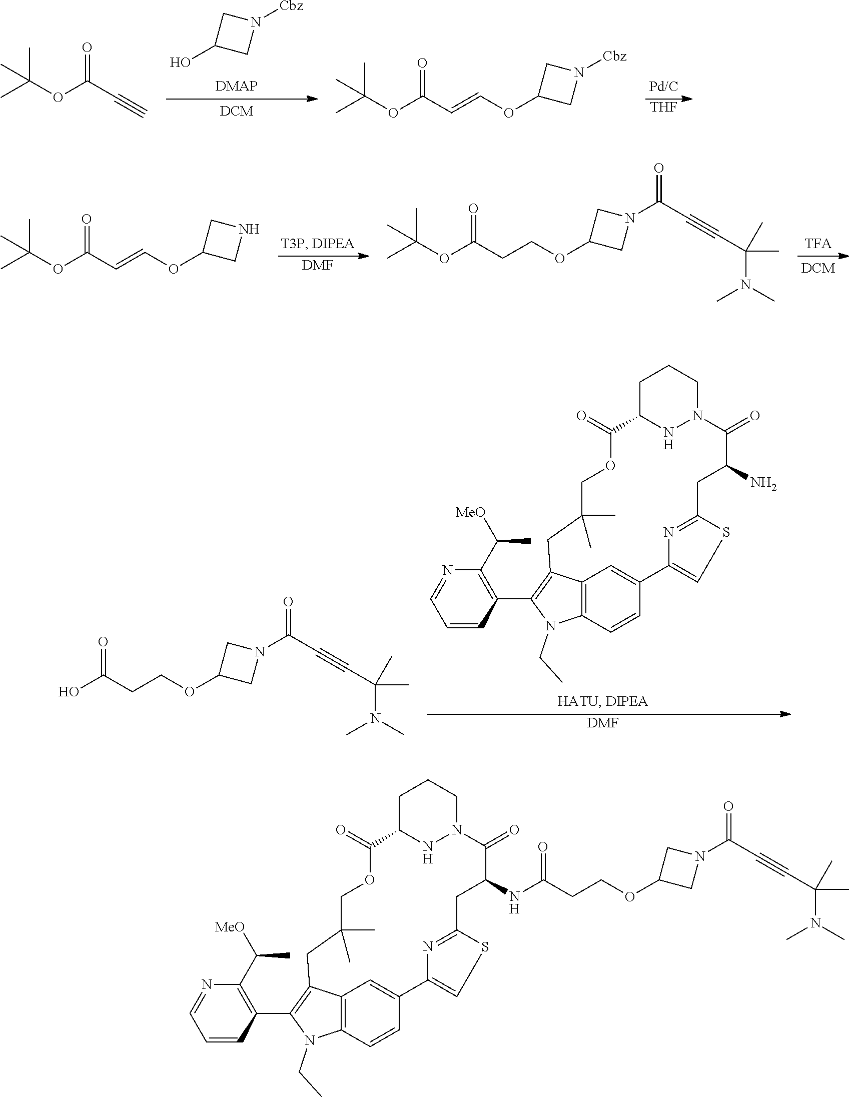

Step 1. To a mixture of tert-butyl prop-2-ynoate (5 g, 40 mmol) and [3-(3-hydroxyazetidin-1-yl)phenyl]methyl formate (4.1 g, 20 mmol) in DCM (150 mL) was added DMAP (9.8 g, 80 mmol). The mixture was stirred for 2 h, then diluted with H₂O and washed with H₂O (60 mL×3). The organic layer was dried over Na₂SO₄, filtered, the filtrate was concentrated under reduced pressure and the residue purified by silica gel column chromatography to give benzyl (E)-3-((3-(tert-butoxy)-3-oxoprop-1-en-1-yl)oxy) azetidine-1-carboxylate (6.6 g, 90% yield) as an oil. LCMS (ESI): m/z [M+Na] calc'd for C₁₈H₂₃NO₅Na 356.2; found 356.2.

Step 2. A mixture of benzyl (E)-3-((3-(tert-butoxy)-3-oxoprop-1-en-1-yl)oxy) azetidine-1-carboxylate (1.4 g, 4 mmol) and Pd/C (200 mg) in THF (10 mL) was stirred under an atmosphere of H₂ (1 atmosphere) for 16 h. The mixture was filtered and the filtrate and was concentrated under reduced pressure to give tert-butyl 3-(azetidin-3-yloxy) propanoate, which was used directly in the next step. LCMS (ESI): m/z [M+H] calc'd for C₁₀H₁₉NO₃ 201.1; found 202.2.

Step 3. To a mixture of tert-butyl 3-(azetidin-3-yloxy) propanoate (300 mg, 1.5 mmol) and 4-(dimethylamino)-4-methylpent-2-ynoic acid (2.3 g, 15 mmol) in DMF (15 mL) at 5° C. was added DIPEA (1.9 g, 15 mmol) and T3P (4.77 g, 7.5 mmol) dropwise. The mixture was stirred at 5° C. for 2 h, then H₂O and EtOAc (80 mL) were added. The organic and aqueous layers were separated and the organic layer was washed with H₂O (20 mL×3), brine (30 mL), dried over anhydrous Na₂SO₄ and filtered. The filtrate was concentrated under reduced pressure and the residue was purified by preparative-HPLC to afford tert-butyl 3-((1-(4-(dimethylamino)-4-methylpent-2-ynoyl) azetidin-3-yl)oxy) propanoate (60 mg, 12% yield) as an oil. LCMS (ESI): m/z [M+H] calc'd for C₁₈H₃₀N₂O₄ 338.2; found 339.2.

Step 4. A mixture tert-butyl 3-((1-(4-(dimethylamino)-4-methylpent-2-ynoyl) azetidin-3-yl)oxy) propanoate (70 mg, 0.21 mmol) in TFA/DCM (1:3, 2 mL) was stirred at 0-5° C. for 1 h, then concentrated under reduced pressure to give 3- ({1-[4-(dimethylamino)-4-methylpent-2-ynoyl)azetidin-3-yl}oxy) propanoic acid (56 mg, 95% yield) as a solid. LCMS (ESI): m/z [M+H] calc'd for C₁₄H₂₂N₂O₄ 282.2; found 283.3.

Step 5. To a mixture of 3-((1-(4-(dimethylamino)-4-methylpent-2-ynoyl) azetidin-3-yl)oxy) propanoic acid (56 mg, 0.19 mmol), (6³S,4S,Z)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (90 mg, 0.14 mmol) and DIPEA (200 mg, 1.9 mmol) in DMF (1 mL) at 0° C. was added HATU (110 mg, 0.38 mmol) portion-wise. The mixture was stirred at 0° C. for 1 h, then H₂O added and the mixture extracted with EtOAx (150 mL×2). The combined organic layers were washed with H₂O (150 mL) and brine (150 mL), then dried over anhydrous Na₂SO₄ and filtered. The filtrate was concentrated under reduced pressure and the residue was purified by preparative-HPLC to give 3-((1-(4-(dimethylamino)-4-methylpent-2-ynoyl) azetidin-3-yl)oxy)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl) propanamide (12.6 mg, 7.5% yield) as a solid. LCMS (ESI): m/z [M+H] calc'd for C₄₈H₆₂N₈O₇S 894.5; found 895.3; ¹H NMR (400 MHZ, CD₃OD) δ 8.73 (dd, J=4.8, 1.6 Hz, 1H), 8.57 (s, 1H), 8.28 (s, 0.3H), 7.84 (m, 1H), 7.71 (m, 1H), 7.52 (m, 3H), 5.76 (dd, J=30.2, 7.8 Hz, 1H), 4.40 (m, 4H), 4.32-4.12 (m, 4H), 4.06 (dd, J=12.4, 6.0 Hz, 1H), 3.97-3.86 (m, 1H), 3.79-3.66 (m, 4H), 3.46 (dd, J=14.8, 4.8 Hz, 1H), 3.41-3.33 (m, 3H), 3.29-3.19 (m, 1H), 3.17-3.05 (m, 1H), 2.79 (m, 1H), 2.73-2.50 (m, 3H), 2.49-2.43 (m, 3H), 2.38 (s, 3H), 2.21 (dd, J=12.6, 9.6 Hz, 1H), 1.95 (d, J=12.8 Hz, 1H), 1.86-1.73 (m, 1H), 1.61 (dd, J=12.6, 3.6 Hz, 1H), 1.51 (s, 2H), 1.46-1.43 (m, 4H), 1.38-1.27 (m, 3H), 1.01-0.86 (m, 6H), 0.44 (d, J=11.6 Hz, 3H).

### Example A716. Synthesis of (3S)-1-acryloyl-N-((2S)-1-(((6³S,4S)-1¹-ethyl-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl) amino)-3-methyl-1-oxobutan-2-yl)-N-methylpyrrolidine-3-carboxamide

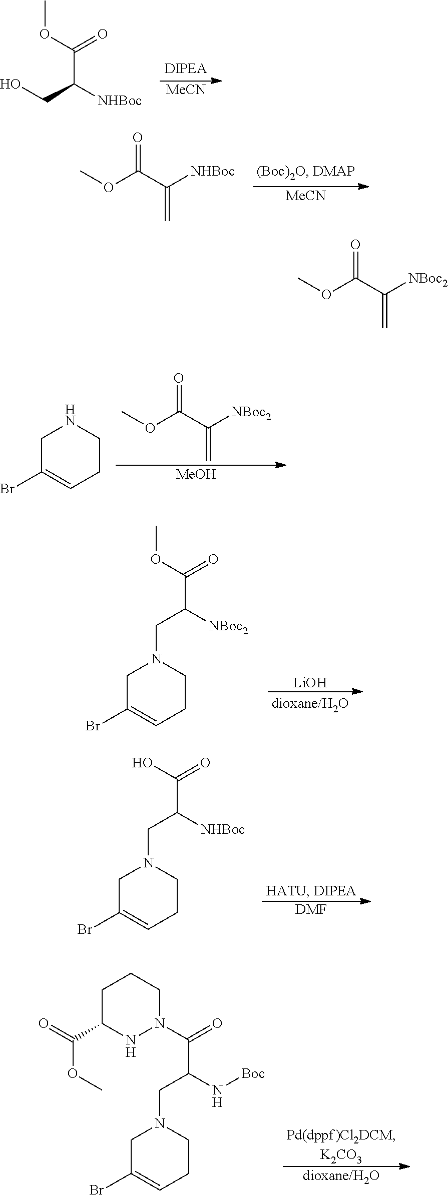

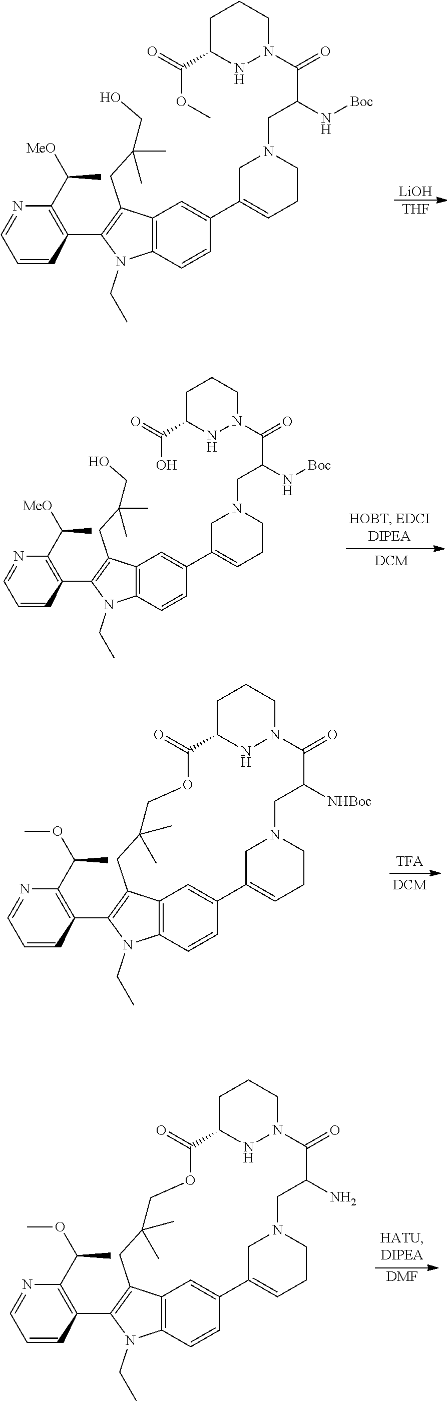

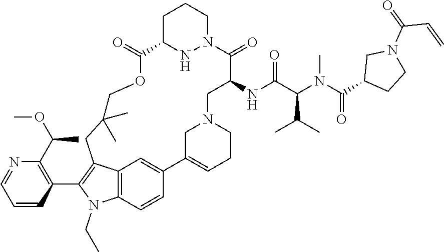

Step 1. To a solution of methyl (tert-butoxycarbonyl)-L-serinate (10 g, 45 mmol) in anhydrous MeCN (150 mL), was added DIPEA (17 g, 137 mmol). The reaction mixture was stirred at 45° C. for 2 h to give methyl 2-((tert-butoxycarbonyl) amino) acrylate in solution. LCMS (ESI): m/z [M+Na] calc'd for C₉H₁₅NO₄ 201.1; found 224.1.

Step 2. To a solution of methyl 2-((tert-butoxycarbonyl) amino) acrylate (12 g, 60 mmol) in anhydrous MeCN (150 mL) at 0° C., was added 4-DMAP (13 g, 90 mmol) and (Boc)₂O (26 g, 120 mmol). The reaction was stirred for 6 h, then quenched with H₂O (100 mL) and extracted with DCM (200 mL×3). The combined organic layers were washed with brine (150 mL), dried over anhydrous sodium sulfate, filtered and concentrated under reduced pressure. The residue was purified by silica gel column chromatography to give methyl 2-(bis (tert-butoxycarbonyl) amino) acrylate (12.5 g, 65% yield) as solid. LCMS (ESI): m/z [M+Na] calc'd for C₁₄H₂₃NO₆ 301.2; found 324.1.

Step 3. To a mixture of 5-bromo-1,2,3,6-tetrahydropyridine (8.0 g, 49 mmol) in MeOH (120 mL) under an atmosphere of Ar was added methyl 2-{bis [(tert-butoxy) carbonyl]amino}prop-2-enoate (22 g, 74 mmol). The mixture was stirred for 16 h, then concentrated under reduced pressure and the residue was purified by silica gel column chromatography to give methyl 2-(bis (tert-butoxycarbonyl) amino)-3-(5-bromo-3,6-dihydropyridin-1 (2H)-yl) propanoate (12 g, 47% yield) as an oil. LCMS (ESI): m/z [M+H] calc'd for C₁₉H₃₁BrN₂O₆ 462.1; found 463.1.

Step 4. To a mixture of methyl 2-(bis (tert-butoxycarbonyl) amino)-3-(5-bromo-3,6-dihydropyridin-1 (2H)-yl) propanoate (14 g, 30 mmol) in 1,4-dioxane (30 mL) and H₂O (12 mL) was added LiOH (3.6 g, 151 mmol). The mixture was heated to 35° C. and stirred for 12 h, then 1M HCl was added and the pH adjusted to ˜3-4. The mixture was extracted with DCM (300 ml×2) and the combined organic layers were dried over anhydrous Na₂SO₄ and filtered. The filtrate was concentrated under reduced pressure to give 3-(5-bromo-3,6-dihydropyridin-1 (2H)-yl)-2-((tert-butoxycarbonyl) amino) propanoic acid (10 g, 85% yield) as a solid. LCMS (ESI): m/z [M+H] calc'd for C₁₃H₂₁BrN₂O₄ 348.1; found 349.0.

Step 5. To a mixture of 3-(5-bromo-3,6-dihydropyridin-1 (2H)-yl)-2-((tert-butoxycarbonyl) amino) propanoic acid (10 g, 30 mmol), DIPEA (12 g, 93 mmol) and methyl (3S)-1,2-diazinane-3-carboxylate (5.4 g, 37 mmol) in DMF (100 mL) at 0° C. under an atmosphere of Ar was added HATU (13 g, 34 mmol). The mixture was stirred at 0° C. for 2 h, then H₂O added and the mixture extracted with EtOAc (300 mL×2). The combined organic layers were dried over anhydrous Na₂SO₄, filtered, the filtrate was concentrated under reduced pressure and the residue was purified by preparative-HPLC to give methyl (3S)-1-(3-(5-bromo-3,6-dihydropyridin-1 (2H)-yl)-2-((tert-butoxycarbonyl) amino) propanoyl) hexahydropyridazine-3-carboxylate (9.0 g, 55% yield) as a solid. LCMS (ESI): m/z [M+H] calc'd for C₁₉H₃₁BrN₄O₅ 474.1; found 475.1.

Step 6. A mixture of methyl (3S)-1-(3-(5-bromo-3,6-dihydropyridin-1 (2H)-yl)-2-((tert-butoxycarbonyl) amino) propanoyl) hexahydropyridazine-3-carboxylate (9.0 g, 18 mmol), K₂CO₃ (4.5 g, 32 mmol), Pd(dppf)Cl₂·DCM (1.4 g, 2 mmol), 3-(1-ethyl-2-{2-[(1S)-1-methoxyethyl]pyridin-3-yl}-5-(4,4,5,5-tetramethyl-1,3,2-dioxaborolan-2-yl) indol-3-yl)-2,2-dimethylpropan-1-ol (9.8 g, 20 mmol) in 1,4-dioxane (90 mL) and H₂O (10 mL) under an atmosphere of Ar was heated to 75° C. and stirred for 2 h. H₂O was added and the mixture was extracted with EtOAc (200 mL×3). The combined organic layers were dried over Na₂SO₄, filtered, the filtrate was concentrated under reduced pressure and the residue was purified by silica gel column chromatography to give methyl (3S)-1-(2-((tert-butoxycarbonyl) amino)-3-(5-(1-ethyl-3-(3-hydroxy-2,2-dimethylpropyl)-2-(2-((S)-1-methoxyethyl) pyridin-3-yl)-1H-indol-5-yl)-3,6-dihydropyridin-1 (2H)-yl) propanoyl) hexahydropyridazine-3-carboxylate (4.0 g, 25% yield) as a solid. LCMS (ESI): m/z [M+H] calc'd for C₄₂H₆₀N₆O₇ 760.5; found 761.4.

Step 7. To a mixture of methyl (3S)-1-(2-((tert-butoxycarbonyl) amino)-3-(5-(1-ethyl-3-(3-hydroxy-2,2-dimethylpropyl)-2-(2-((S)-1-methoxyethyl) pyridin-3-yl)-1H-indol-5-yl)-3,6-dihydropyridin-1 (2H)-yl) propanoyl) hexahydropyridazine-3-carboxylate (4.1 g, 5.0 mmol) in THF (35 mL) at 0° C. was added LiOH (0.60 g, 27 mmol). The mixture was stirred at 0° C. for 1.5 h, then 1M HCl added to adjust pH to ˜6-7 and the mixture extracted with EtOAc (200 mL×3). The combined organic layers were dried over Na₂SO₄, filtered and the filtrate was concentrated under reduced pressure to give (3S)-1-(2-((tert-butoxycarbonyl) amino)-3-(5-(1-ethyl-3-(3-hydroxy-2,2-dimethylpropyl)-2-(2-((S)-1-methoxyethyl) pyridin-3-yl)-1H-indol-5-yl)-3,6-dihydropyridin-1 (2H)-yl) propanoyl) hexahydropyridazine-3-carboxylic acid (3.6 g, 80% yield) as a solid. LCMS (ESI): m/z [M+H] calc'd for C₄₁H₅₈N₆O₇ 746.4; found 747.4.

Step 8. To a mixture of (3S)-1-(2-((tert-butoxycarbonyl) amino)-3-(5-(1-ethyl-3-(3-hydroxy-2,2-dimethylpropyl)-2-(2-((S)-1-methoxyethyl) pyridin-3-yl)-1H-indol-5-yl)-3,6-dihydropyridin-1 (2H)-yl) propanoyl) hexahydropyridazine-3-carboxylic acid (3.6 g, 5.0 mmol) and DIPEA (24 g, 190 mmol) in DCM (700 mL) under an atmosphere of Ar was added EDCI·HCl (28 g, 140 mmol) and HOBT (6.5 g, 50 mmol). The mixture was heated to 30° C. and stirred for 16 h at 30° C., then concentrated under reduced pressure. The residue was diluted with EtOAc (200 mL) and washed with H₂O (200 mL×2), brine (200 mL), dried over Na₂SO₄ and filtered. The filtrate was concentrated under reduced pressure and the residue was purified by silica gel column chromatography to give tert-butyl ((63S)-1¹-ethyl-1²-(2-((S)-1-methoxyethyl) pyridin-3-yl)-10, 10-dimethyl-5,7-dioxo-21,22,23,26,61,62,63,64,65,66-decahydro-1¹H-8-oxa-1 (5,3)-indola-6 (1,3)-pyridazina-2 (5,1)-pyridinacycloundecaphane-4-yl) carbamate (1.45 g, 40% yield) as a solid. LCMS (ESI): m/z [M+H] calc'd for C₄₁H₅₆N₆O₆ 728.4; found 729.4.

Step 9. To a mixture of tert-butyl ((63S)-1¹-ethyl-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)- pyridazina-2 (5, 1)-pyridinacycloundecaphane-4-yl) carbamate (130 mg, 0.20 mmol) in DCM (1.0 mL) at 0° C. was added TFA (0.3 mL). The mixture was warmed to room temperature and stirred for 2 h, then concentrated under reduced pressure to give (63S)-4-amino-1¹-ethyl-1²-(2-((S)-1-methoxyethyl) pyridin-3-yl)-10, 10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-5,7-dione, which was used directly in the next step directly without further purification. LCMS (ESI): m/z [M+H] calc'd for C₃₆H₄₈N₆O₄ 628.4; found 629.4.

Step 10. To a mixture of ((63S)-4-amino-1¹-ethyl-1²-(2-((S)-1-methoxyethyl) pyridin-3-yl)-10,10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-5,7-dione (130 mg, 0.2 mmol), DIPEA (270 mg, 2.0 mmol) and (2S)-3-methyl-2-{N-methyl-1-[(3S)-1-(prop-2-enoyl) pyrrolidin-3-yl]formamido}butanoic acid (118 mg, 0.40 mmol) in DMF (3.0 mL) at 0° C. under an atmosphere of Ar was added HATU (87 mg, 0.30 mmol) in portions. The mixture was stirred at 0° C. for 1 h, then diluted with H₂O extracted with EtOAc (30 mL×2). The combined organic layers were dried over anhydrous Na₂SO₄, filtered, the filtrate was concentrated under reduced pressure and the residue was purified by preparative-HPLC to give (3S)-1-acryloyl-N-((2S)-1-(((6³S,4S)-1¹-ethyl-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)- pyridazina-2 (5, 1)-pyridinacycloundecaphane-4-yl) amino)-3-methyl-1-oxobutan-2-yl)-N-methylpyrrolidine-3-carboxamide (17.2 mg, 10% yield) as a solid. LCMS (ESI): m/z [M+H] calc'd for C₅₀H₆₈N₆O₄ 892.5; found 893.5; ¹H NMR (400 MHZ, CD₃OD) δ 8.74 (d, J=4.4 Hz, 1H), 7.93-7.90 (m, 1H), 7.56-7.51 (m, 3H), 7.43 (d, J=4.4 Hz, 1H), 6.63-6.53 (m, 2H), 6.33-6.23 (m, 2H), 5.83-5.70 (m, 1H), 4.73-4.70 (d, J=11.0 Hz, 1H), 4.48-4.45 (d, J=13.0 Hz, 1H), 4.12-4.10 (m, 3H), 3.86-3.81 (m, 4H), 3.79-3.75 (m, 1H), 3.72-3.69 (m, 3H), 3.57-3.47 (m, 2H), 3.21-3.09 (m, 1H), 3.07-3.04 (q, 4H), 3.02-2.95 (m, 3H), 2.86-2.82 (m, 3H), 2.66-2.48 (m, 2H), 2.29-2.17 (m, 4H), 2.11-1.98 (m, 2H), 1.95-1.91 (m, 1H), 1.45 (d, J=6.2 Hz, 3H), 1.23-1.16 (m, 2H), 1.09-1.04 (m, 1H), 0.97-0.93 (m, 3H), 0.92-0.81 (m, 5H), 0.67-0.63 (m, 3H).

### Example A663. The synthesis of (2R)-2-(((1-(4-(dimethylamino)-4-methylpent-2-ynoyl) 373yridine373-3-yl)oxy) methyl)-N-((6³S,4S)-1¹-ethyl-1²-(2-((S)-1-methoxyethyl) 373yridine-3-yl)-10, 10-dimethyl-5,7-dioxo-2¹,2²,2³,2⁶,6¹,6²,6³,6⁴,6⁵,6⁶-decahydro-11H-8-oxa-1 (5,3)-indola-6 (1,3)-pyridazina-2 (5,1)-pyridinacycloundecaphane-4-yl)-3-methylbutanamide

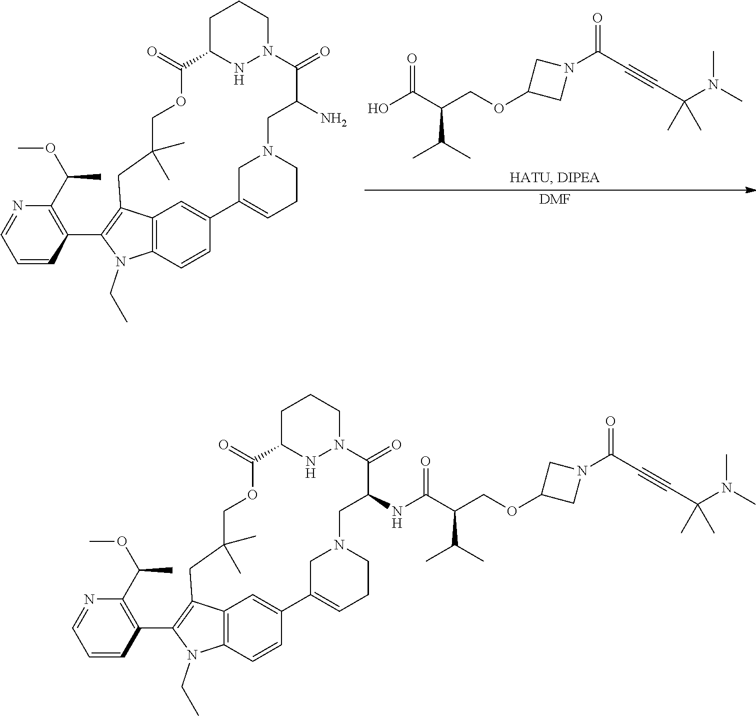

To a mixture of (6³S,4S)-4-amino-1¹-ethyl-1²-(2-((S)-1-methoxyethyl) 373yridine-3-yl)-10,10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)- pyridinacycloundecaphane-5,7-dione (100 mg, 0.16 mmol),®-2-(((1-(4-(dimethylamino)-4-methylpent-2-ynoyl) 373yridine373-3-yl)oxy) methyl)-3-methylbutanoic acid (80 mg, 0.24 mmol) and DIPEA (825 mg, 6.4 mmol) in DMF (2 mL) at 0° C., was added HATU (95 mg, 0.24 mmol). The reaction mixture was stirred at 0° C. for 1 h, then poured into H₂O (60 mL), extracted with EtOAc (80 mL×2). The combined organic layers were washed with H₂O (80 mL) and brine (80 mL), dried over anhydrous sodium sulfate, filtered and concentrated under reduced pressure. The residue was purified by silica gel chromatography to afford (2R)-2-(((1-(4-(dimethylamino)-4-methylpent-2-ynoyl) 373yridine373-3-yl)oxy) methyl)-N-((6³S,4S)-1¹-ethyl-1²-(2-((S)-1-methoxyethyl) 373yridine-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)- pyridazina-2 (5,1)-pyridinacycloundecaphane-4-yl)-3-methylbutanamide (55 mg, 36% yield) as solid. ¹H NMR (400 MHZ, CD₃OD) δ 8.76-8.70 (m, 1H), 8.49 (dd, J=4.3, 1.4 Hz, 0.1H), 7.93-7.87 (m, 1H), 7.58-7.50 (m, 3H), 7.41 (dd, J=8.8, 3.2 Hz, 1H), 6.26 (d, J=16.8 Hz, 1H), 5.96 (t, J=9.6 Hz, 1H), 4.47 (d, J=12.8 Hz, 1H), 4.39-4.28 (m, 2H), 4.21-3.97 (m, 5H), 3.96-3.70 (m, 5H), 3.68-3.54 (m, 3H), 3.51-3.35 (m, 1H), 3.11 (d, J=22.7 Hz, 3H), 3.00-2.67 (m, 5H), 2.46-2.30 (m, 7H), 2.24 (s, 3H), 2.11 (d, J=12.4 Hz, 1H), 1.92 (d, J=13.2 Hz, 1H), 1.85-1.60 (m, 3H), 1.45 (d, J=7.8 Hz, 6H), 1.32 (d, J=16.0 Hz, 3H), 1.12 (dt, J=24.5, 6.8 Hz, 3H), 0.95 (m, 6H), 0.76 (m, 6H). LCMS (ESI): m/z [M+H] calc'd for C₅₃H₇₄N₈O₇ 934.6; found 935.5.

### Example A646. The synthesis of (2R)-2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (3,1)-piperidinacycloundecaphane-4-yl)-3-methylbutanamide

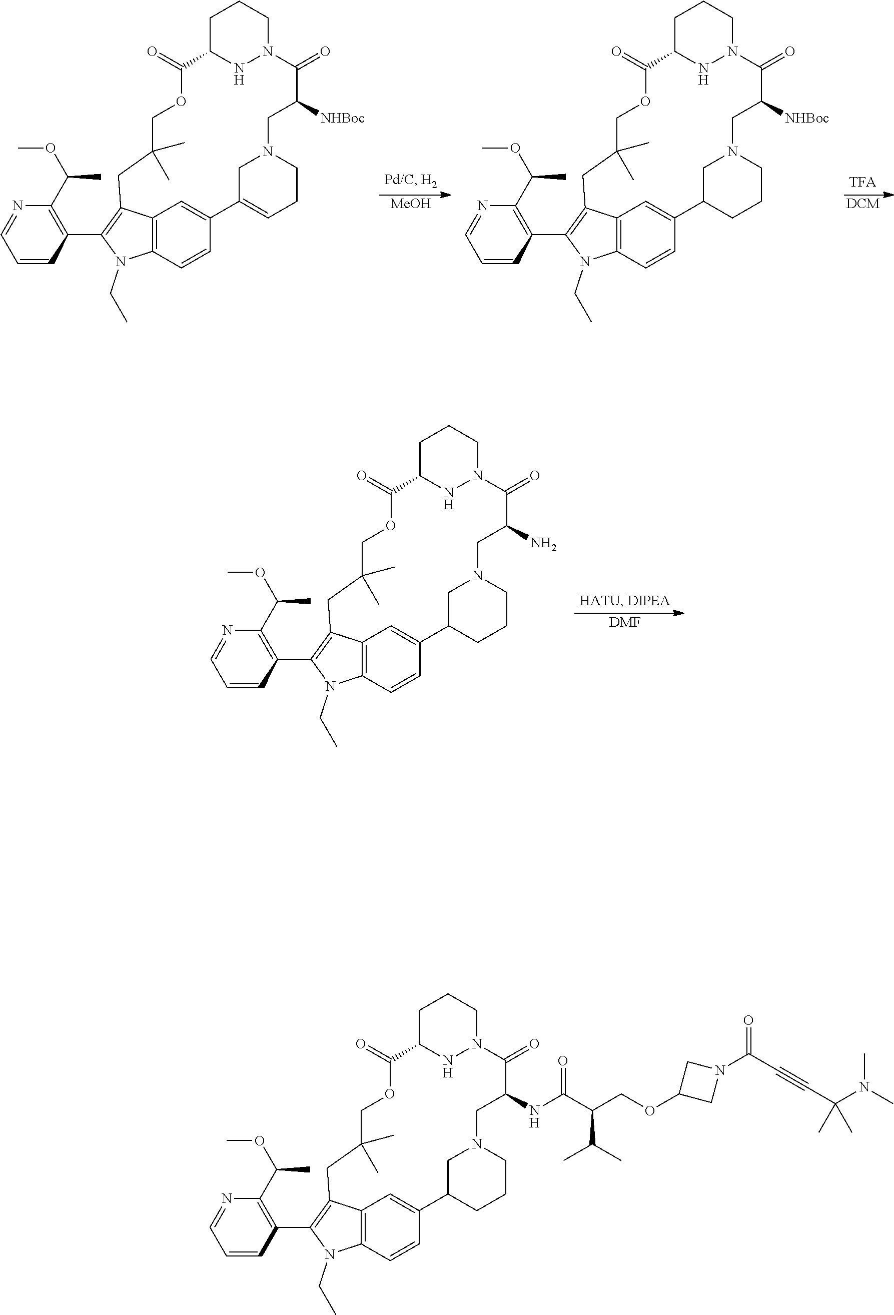

Step 1. A mixture of tert-butyl ((63S)-1¹-ethyl-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl) carbamate (0.2 g, 0.28 mmol) and Pd/C (0.2 g, 2 mmol) in MeOH (10 mL) was stirred at 25° C. for 16 h under an H₂ atmosphere. The reaction mixture was filtered through Celite, concentrated under reduced pressure to afford tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (3,1)-piperidinacycloundecaphane-4-yl) carbamate as solid. LCMS (ESI): m/z [M+H] calc'd for C₄₁H₅₈N₆O₆ 730.4; found 731.4. Step 2. To a solution of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (3,1)-piperidinacycloundecaphane-4-yl) carbamate (150 mg, 0.2 mmol) in DCM (1.5 mL) at 0° C. was added TFA (0.5 mL). The reaction mixture was stirred at 20° C. for 1 h, then concentrated under reduced pressure to afford (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (3,1)-piperidinacycloundecaphane-5,7-dione as solid. LCMS (ESI): m/z [M+H] calc'd for C₃₆H₅₀N₆O₄ 630.4; found 631.4.

Step 3. To a mixture of (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (3,1)-piperidinacycloundecaphane-5,7-dione (240 mg, 0.4 mmol), DIPEA (982 mg, 2 mmol) and (R)-2-(((1-(4-(dimethylamino)-4-methylpent-2-ynoyl) azetidin-3-yl)oxy) methyl)-3-methylbutanoic acid (148 mg, 0.45 mmol) in DMF (4 mL) at 0° C. under argon atmosphere, was added HATU (173 mg, 0.46 mmol) in portions. The reaction mixture was stirred at 0° C. under an argon atmosphere for 1 h, then quenched with H₂O at 0° C. The resulting mixture was extracted with EtOAc (30 ml×2). The combined organic layers were dried over anhydrous sodium sulfate, filtered and concentrated under reduced pressure. The residue was purified by reverse phase chromatography to afford (2R)-2-(((1-(4-(dimethylamino)-4-methylpent-2-ynoyl) azetidin-3-yl)oxy) methyl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (3,1)-piperidinacycloundecaphane-4-yl)-3-methylbutanamide (150 mg, 38% yield) as solid. ¹H NMR (400 MHZ, CD₃OD) δ 8.72 (d, J=4.8 Hz, 1H), 7.88 (d, J=8.0 Hz, 1H), 7.53-7.49 (m, 2H), 7.42 (d, J=8.4 Hz, 1H), 7.18 (d, J=8.4 Hz, 1H), 5.95-5.91 (m, 1H), 4.52-4.49 (m, 1H), 4.37-4.25 (m, 3H), 4.18-4.15 (m, 2H), 3.99-3.98 (m, 2H), 3.90-3.86 (m, 1H), 3.76-3.68 (m, 2H), 3.55-3.50 (m, 2H), 3.39-3.36 (m, 2H), 3.20 (s, 3H), 3.02 (s, 3H), 2.89-2.79 (m, 3H), 2.62-2.50 (m, 2H), 2.36 (s, 3H), 2.35-2.30 (m, 1H), 2.26 (s, 3H), 2.20-1.15 (m, 1H), 1.97-1.93 (m, 3H), 1.81-1.76 (m, 4H), 1.64-1.61 (m, 2H), 1.46-1.43 (m, 6H), 1.36 (d, J=14.8 Hz, 3H), 1.02 (s, 3H), 0.94 (m, 6H), 0.81 (s, 3H), 0.65 (s, 3H). LCMS (ESI): m/z [M+H] calc'd for C₅₃H₇₆N₈O₇ 936.6; found 937.5.

### Example A740. Synthesis of (3S)-1-acryloyl-N-((2S)-1-(((23S,6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (3,1)-piperidinacycloundecaphane-4-yl) amino)-3-methyl-1-oxobutan-2-yl)-N-methylpyrrolidine-3-carboxamide

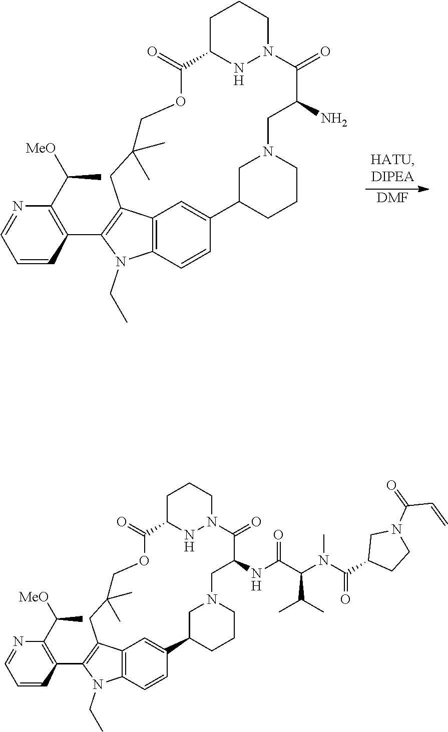

To a mixture of (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (3,1)-piperidinacycloundecaphane-5,7-dione (140 mg, 0.20 mmol), DIPEA (570 mg, 4.4 mmol) and (2S)-3-methyl-2-{N-methyl-1-[(3S)-1-(prop-2-enoyl) pyrrolidin-3-yl]formamido}butanoic acid (124 mg, 0.40 mmol) in DMF (3.0 mL) at 0° C. under an atmosphere of Ar was added HATU (100 mg, 0.30 mmol) in portions. The mixture was stirred at 0° C. for 1 h, then H₂O was added and the mixture extracted with EtOAc (2×30 mL). The combined organic layers were dried over anhydrous Na₂SO₄ and filtered. The filtrate was concentrated under reduced pressure and the residue was purified by preparative-HPLC to give (3S)-1-acryloyl-N-((2S)-1-(((23S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (3, 1)-piperidinacycloundecaphane-4-yl) amino)-3-methyl-1-oxobutan-2-yl)-N-methylpyrrolidine-3-carboxamide (41 mg, 20% yield) as a solid. LCMS (ESI): m/z [M+H] calc'd for C₅₀H₇₀N₈O₇ 894.5; found 895.5; ¹H NMR (400 MHZ, CD₃OD) δ 8.72 (d, J=4.8 Hz, 1H), 7.87 (d, J=7.6 Hz, 1H), 7.51-7.49 (m, 2H), 7.42-7.37 (m, 1H), 7.18-7.14 (m, 1H), 6.64-6.54 (m, 1H), 6.30-6.23 (m, 1H), 5.77-5.70 (m, 2H), 4.65-4.60 (m, 1H), 4.50-4.40 (m, 1H), 4.27-4.16 (m, 2H), 4.00-3.95 (m, 2H), 3.83-3.78 (m, 2H), 3.73-3.60 (m, 4H), 3.51-3.36 (m, 3H), 3.22-3.19 (m, 4H), 3.07 (d, J=6.8 Hz, 2H), 2.99 (d, J=12.0 Hz, 3H), 2.90-2.78 (m, 2H), 2.75-2.64 (m, 3H), 2.20-2.10 (m, 4H), 2.02-1.93 (m, 3H), 1.87-1.64 (m, 4H), 1.45 (d, J=4.8 Hz, 3H), 1.06-1.00 (m, 4H), 0.97-0.89 (m, 3H), 0.83-0.79 (m, 3H), 0.66 (s, 3H).

### Example A534. (2S)-2-((S)-7-(4-(dimethylamino)-4-methylpent-2-ynoyl)-1-oxo-2,7-diazaspiro[4.4]nonan-2-yl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

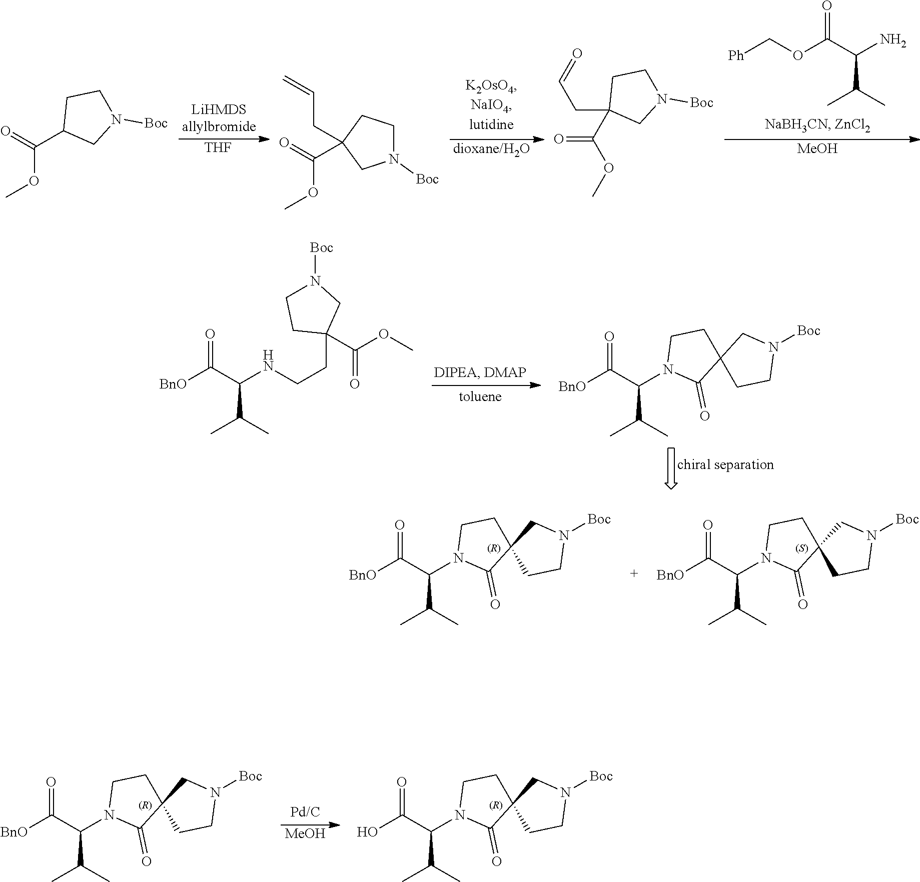

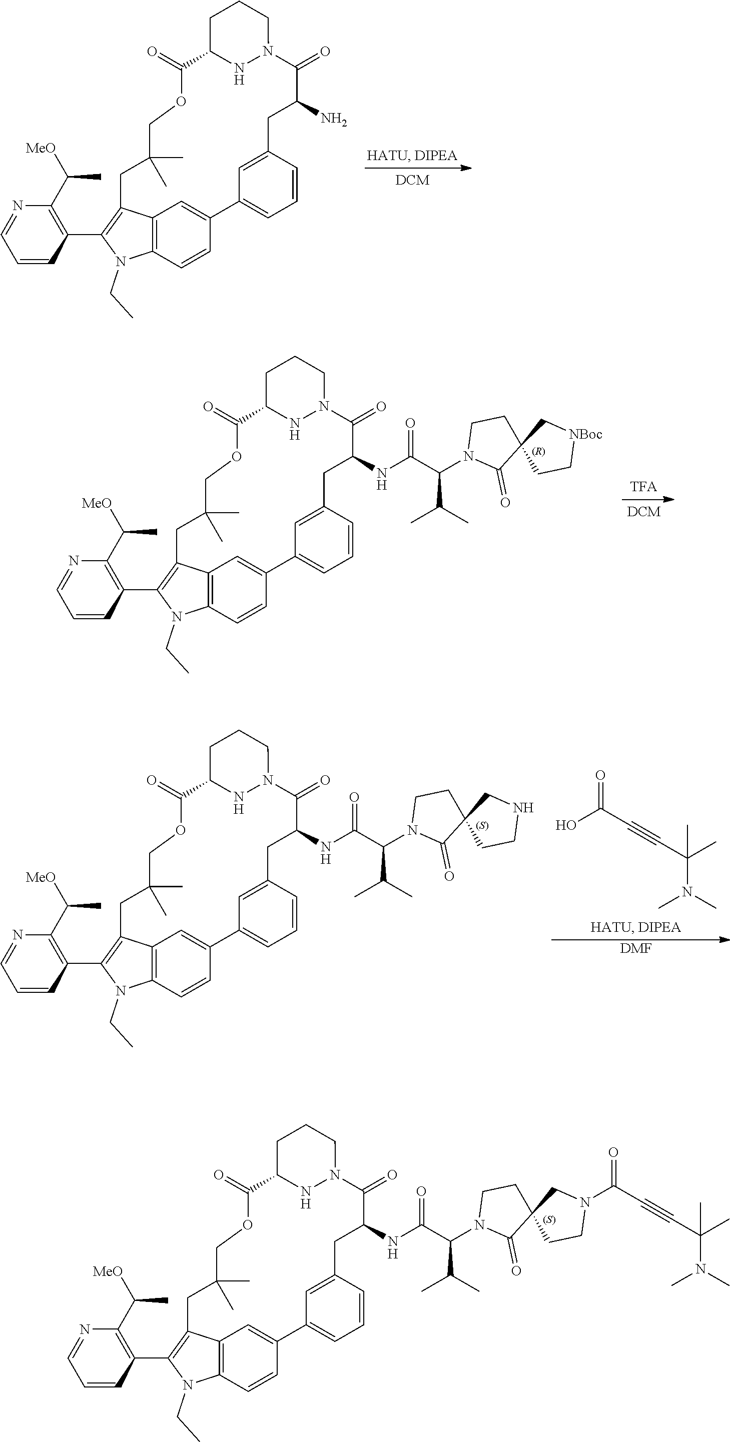

Step 1. To a mixture of 1-tert-butyl 3-methyl pyrrolidine-1,3-dicarboxylate (20.0 g, 87.2 mmol) in THF (150 mL) at −78° C. under an atmosphere of nitrogen was added 1M LiHMDS in THF (113.4 mL, 113.4 mmol). After stirring at −78° C. for 40 min, allyl bromide (13.72 g, 113.4 mmol) was added and the mixture was allowed to warm to room temperature and stirred for 4 h. The mixture was cooled to 0° C., saturated NaCl (30 mL) was added and the mixture extracted with EtOAc. The combined organic layers were dried over anhydrous Na₂SO₄ and filtered. The filtrate was concentrated under reduced pressure and the residue was purified by silica gel column chromatography to give 1-(tert-butyl) 3-methyl 3-allylpyrrolidine-1,3-dicarboxylate (17 g, 72% yield) as an oil. ¹H NMR (300 MHZ, CDCl₃) δ 5.80-5.60 (m, 1H), 5.16-5.02 (m, 2H), 3.71 (s, 4H), 3.42 (d, J=9.3 Hz, 2H), 3.27 (t, J=11.2 Hz, 1H), 2.42 (d, J=7.6 Hz, 2H), 2.38-2.24 (m, 1H), 2.05 (s, 1H), 1.85 (dt, J=14.3, 7.5 Hz, 1H), 1.46 (s, 10H), 1.27 (t, J=7.1 Hz, 1H).

Step 2. To a mixture of 1-(tert-butyl) 3-methyl 3-allylpyrrolidine-1,3-dicarboxylate (4.0 g, 14.9 mmol) and 2,6-dimethylpyridine (3.18 g, 29.7 mmol) in 1,4-dioxane (200 mL) and H₂O (100 mL) at 0° C. was added K₂OsO₄ 2H₂O (0.11 g, 0.3 mmol) in portions. The mixture was stirred for 15 min at 0° C., then NaIO₄ (6.35 g, 29.7 mmol) was added in portions. The mixture was stirred at room temperature for 3 h at room temperature, then cooled to 0° C. and saturated aqueous Na₂S203 (50 mL) added. The mixture was extracted with EtOAc (3×100 mL) and the combined organic layers were washed with 2 M HCl, then dried over anhydrous Na₂SO₄ and filtered. The filtrate was concentrated under reduced pressure to give 1-(tert-butyl) 3-methyl 3-(2-oxoethyl) pyrrolidine-1,3-dicarboxylate (4 g, 52% yield) as an oil. ¹H NMR (300 MHZ, CDCl₃) δ 5.80-5.60 (m, 1H), 5.16-5.04 (m, 2H), 3.72 (s, 3H), 3.41 (s, 3H), 3.28 (d, J=11.0 Hz, 1H), 2.44 (s, 2H), 2.31 (d, J=9.1 Hz, 1H), 1.85 (dt, J=12.7, 7.5 Hz, 1H), 1.69 (s, 1H), 1.47 (s, 10H).

Step 3. To a mixture of 1-(tert-butyl) 3-methyl 3-(2-oxoethyl) pyrrolidine-1,3-dicarboxylate (6.30 g, 23.2 mmol), in MeOH (70 mL) at 0° C. was added benzyl (2S)-2-amino-3-methylbutanoate (7.22 g, 34.8 mmol) and ZnCl₂ (4.75 g, 34.8 mmol). The mixture was warmed to room temperature and stirred for 30 min, then cooled to 0° C. and NaCNBH₃ (2.92 g, 46.4 mmol) was added in portions. The mixture was warmed to room temperature and stirred for 2 h, then cooled to 0° C. and saturated aqueous NH₄Cl added. The mixture was extracted with EtOAc (3×200 mL) and the combined organic layers were washed with brine (150 mL), dried over anhydrous Na₂SO₄ and filtered. The filtrate was concentrated under reduced pressure and the residue was purified by silica gel column chromatography to give 1-(tert-butyl) 3-methyl 3-(2-(((S)-1-(benzyloxy)-3-methyl-1-oxobutan-2-yl) amino) ethyl) pyrrolidine-1,3-dicarboxylate (6.4 g, 54% yield) as an oil. LCMS (ESI): m/z [M+H] calc'd for C₂₅H₃₈N₂O₆ 462.3; found 463.4.

Step 4. To a mixture of 1-(tert-butyl) 3-methyl 3-(2-(((S)-1-(benzyloxy)-3-methyl-1-oxobutan-2-yl) amino) ethyl) pyrrolidine-1,3-dicarboxylate (4.50 g, 9.7 mmol) in toluene (50 mL) was added DIPEA (12.57 g, 97.3 mmol) and DMAP (1.19 g, 9.7 mmol). The resulting mixture was heated to 80° C. and stirred for 24 h, then concentrated under reduced pressure and the residue was purified by preparative-HPLC, then by chiral-HPLC to give tert-butyl (R)-7-((S)-1-(benzyloxy)-3-methyl-1-oxobutan-2-yl)-6-oxo-2,7-diazaspiro[4.4]nonane-2-carboxylate (1.0 g, 32% yield) and tert-butyl(S)-7-((S)-1-(benzyloxy)-3-methyl-1-oxobutan-2-yl)-6-oxo-2,7-diazaspiro[4.4]nonane-2-carboxylate (1.0 g, 32% yield) and as a an oil. LCMS (ESI): m/z [M+H] calc'd for C₂₄H₃₄N₂O₅ 430.5; found 431.2 and LCMS (ESI): m/z [M+H] calc'd for C₂₄H₃₄N₂O₅ 430.3; found 431.2.

Step 5. A mixture of tert-butyl (R)-7-((S)-1-(benzyloxy)-3-methyl-1-oxobutan-2-yl)-6-oxo-2,7-diazaspiro[4.4]nonane-2-carboxylate (4.0 g) and 10% Pd/C (1 g) in MeOH (40 mL) was stirred at room temperature under an atmosphere of H₂. The mixture was filtered through a pad of Celite pad and the filtrae was concentrated under reduced pressure to give (S)-2-((R)-7-(tert-butoxycarbonyl)-1-oxo-2,7-diazaspiro[4.4]nonan-2-yl)-3-methylbutanoic acid (4.9 g) as a solid. LCMS (ESI): m/z [M−H] calc'd for C₁₇H₂₈N₂O₅ 340.2; found 339.3.

Step 6. To a mixture of (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (500 mg, 0.8 mmol) in DCM at 0° C. were added DIPEA (829 mg, 6.4 mmol), ((S)-2-((R)-7-(tert-butoxycarbonyl)-1-oxo-2,7-diazaspiro[4.4]nonan-2-yl)-3-methylbutanoic acid (273 mg, 0.8 mmol) and HATU (396 mg, 1.0 mmol) in portions over 1 min. The mixture was allowed to warm to room temperature and stirred 2 h, then concentrated under reduced pressure and the residue was purified by preparative-TLC to give tert-butyl (5R)-7-((2S)-1-(((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-6-oxo-2,7-diazaspiro[4.4]nonane-2-carboxylate (500 mg, 64% yield) as an oil. LCMS (ESI): m/z [M+H] calc'd for C₅₄H₇₁N₇O₈ 945.5; found 946.5.

Step 7. To a mixture of tert-butyl (5R)-7-((2S)-1-(((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-6-oxo-2,7-diazaspiro[4.4]nonane-2-carboxylate (1.0 g, 1.06 mmol) in DCM (10 mL) at 0° C. was added TFA (3 mL) dropwise. The mixture was warmed to room temperature and stirred for 1 h, then concentrated under reduced pressure to give (2S)—N-((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-2,7-diazaspiro[4.4]nonan-2-yl) butanamide (1.3 g). LCMS (ESI): m/z [M−H] calc'd for C₄₉H₆₃N₇O₆ 846.1; found 845.5.

Step 8. To a mixture of (2S)—N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-2,7-diazaspiro[4.4]nonan-2-yl) butanamide (500 mg, 0.59 mmol) and DIPEA (764 mg, 5.9 mmol) in DMF (5 mL) at 0° C. were added 4-(dimethylamino)-4-methylpent-2-ynoic acid (110 mg, 0.71 mmol) and HATU (292 mg, 0.77 mmol) in portions. The mixture was warmed to room temperature and stirred for 1 h, then H₂O (10 mL) was added and the mixture extracted with EtOAc (10 mL×3). The combined organic layers were washed with brine (10 mL), dried over anhydrous Na₂SO₄ and filtered. The filtrate was concentrated under reduced pressure and the residue was purified by preparative-HPLC to give (2S)-2-((S)-7-(4-(dimethylamino)-4-methylpent-2-ynoyl)-1-oxo-2,7-diazaspiro[4.4]nonan-2-yl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (177 mg, 28.94% yield) as a white solid. LCMS (ESI): m/z [M+H] calc'd for C₅₇H₇₄N₈O₇ 982.6; found 983.8; ¹H NMR (400 MHz, DMSO-d₆) δ 8.76 (dd, J=4.7, 1.7 Hz, 1H), 8.52 (d, J=7.9 Hz, 1H), 7.99 (d, J=1.7 Hz, 1H), 7.83 (d, J=10.2 Hz, 2H), 7.74-7.58 (m, 3H), 7.53 (dd, J=7.7, 4.8 Hz, 1H), 7.24 (t, J=7.6 Hz, 1H), 7.12 (d, J=7.6 Hz, 1H), 5.32 (d, J=9.7 Hz, 2H), 4.33-4.20 (m, 4H), 4.03 (dd, J=15.0, 8.6 Hz, 2H), 3.88-3.82 (m, 1H), 3.63 (dq, J=20.5, 10.3 Hz, 4H), 3.42-3.34 (m, 2H), 3.21 (s, 1H), 3.13 (d, J=2.8 Hz, 3H), 2.87 (s, 2H), 2.83-2.72 (m, 2H), 2.69-2.62 (m, 1H), 2.21 (d, J=22.6 Hz, 6H), 2.12-1.76 (m, 7H), 1.75-1.47 (m, 2H), 1.46-1.28 (m, 9H), 0.99-0.89 (m, 6H), 0.79-0.71 (m, 6H), 0.52 (s, 3H).

### Example A341. Synthesis of (3S)-1-acryloyl-N-((2S)-1-(((6³S,4S)-25-(difluoromethyl)-11-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

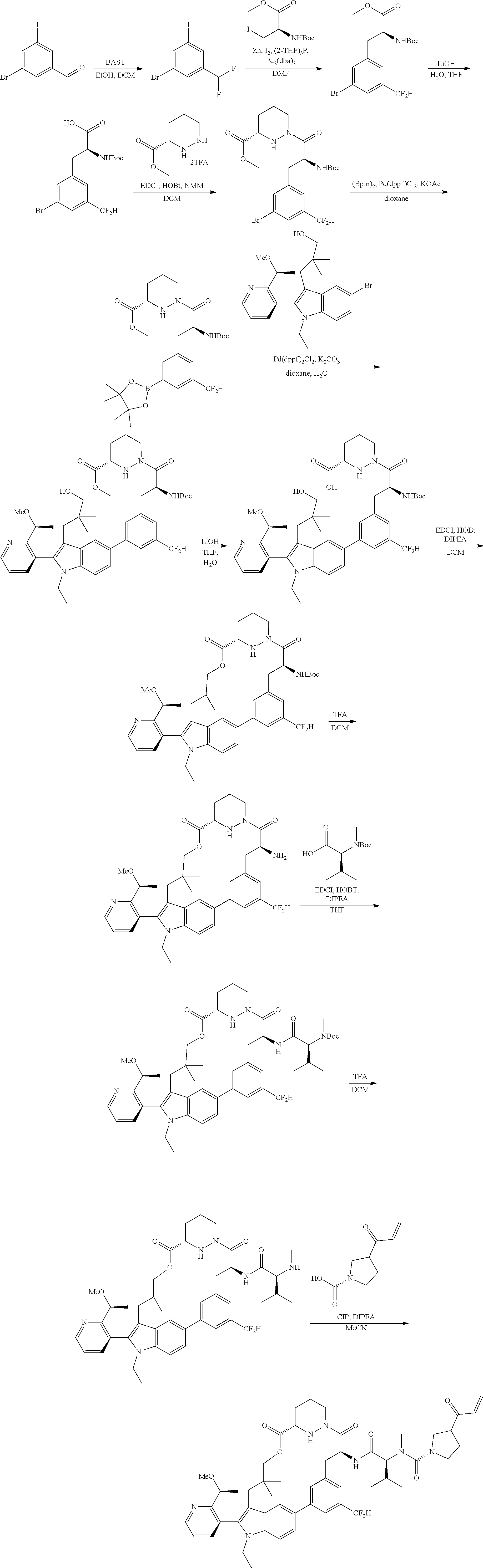

Step 1. To a mixture of 3-bromo-5-iodobenzaldehyde (4.34 g, 14.0 mmol) in DCM at 0° C. under an atmosphere of N₂ was added BAST (6.8 g, 30.7 mmol) and EtOH (129 mg, 2.8 mmol) dropwise. The mixture was heated with microwave heating at 27° C. for 14 h. H₂O (500 mL) was added and the mixture was extracted with DCM (200 mL×3), the combined organic layers were concentrated under reduced pressure and the residue was purified by silica gel column chromatography to give 1-bromo-3-(difluoromethyl)-5-iodobenzene (3.2 g, 65% yield) as a solid. 1H NMR (300 MHZ, DMSO-d₆) δ 8.16 (p, J=1.2 Hz, 1H), 7.94 (p, J=1.3 Hz, 1H), 7.81 (p, J=1.3 Hz, 1H), 7.00 (t, J=55.3 Hz, 1H).

Step 2. A mixture of Zn (2.28 g, 34.8 mmol) and 12 (442 mg, 1.74 mmol) in DMF (20 mL) under an atmosphere of Ar was stirred at 50° C. for 0.5 h. To this mixture was added a solution of methyl (methyl (R)-2-((tert-butoxycarbonyl) amino)-3-iodopropanoate (2.39 g, 7.25 mmol) in DMF (20 mL) and the mixture was stirred at 50° C. for 2 h. After cooling, the mixture was added to 1-bromo-3-(difluoromethyl)-5-iodobenzene (2.90 g, 8.7 mmol), Pd₂(dba)₃ (239 mg, 0.26 mmol) and tri-2-furylphosphine (162 mg, 0.7 mmol) in DMF (20 mL). The mixture was heated to 70° C. and stirred for 2 h, then H₂O (200 mL) was added and the mixture extracted with EtOAc (200 mL×3). The combined organic layers were concentrated under reduced pressure and the residue was purified by silica gel column chromatography to give methyl(S)-3-(3-bromo-5-(difluoromethyl)phenyl)-2-((tert-butoxycarbonyl) amino) propanoate (560 mg, 19% yield) as a solid. ¹H NMR (300 MHZ, DMSO-d₆) δ 7.65 (d, J=10.0 Hz, 2H), 7.47 (s, 1H), 7.36 (d, J=8.4 Hz, 1H), 7.00 (t, J=55.6 Hz, 1H), 4.25 (td, J=9.6, 4.7 Hz, 1H), 3.64 (s, 3H), 3.11 (dd, J=13.6, 4.9 Hz, 1H), 3.00-2.80 (m, 1H), 1.32 (s, 9H).

Step 3. To a mixture of methyl(S)-3-(3-bromo-5-(difluoromethyl)phenyl)-2-((tert-butoxycarbonyl) amino) propanoate (650 mg, 1.6 mmol) in THF (1.5 mL) at 0° C. under an atmosphere of N₂ was added LiOH (114 mg, 4.8 mmol) in H₂O (1.50 mL). The mixture was stirred at 0° C. for 1 h, then acidified to pH 5 with 1M HCl. The mixture was extracted with DCM/MeOH (10/1) (100 mL×3) and the combined organic layers were dried over anhydrous Na₂SO₄ and filtered. The filtrate was concentrated under reduced pressure to give (S)-3-(3-bromo-5-(difluoromethyl)phenyl)-2-((tert-butoxycarbonyl) amino) propanoic acid (500 mg), which was used directly in the next step without further purification. LCMS (ESI): m/z [M+H] calc'd for C₁₅H₁₈BrF₂NO₄ 393.0; found 392.1.

Step 4. To a mixture of methyl (3S)-1,2-diazinane-3-carboxylate (475 mg, 3.3 mmol) in DCM (10 mL) at 0° C. under an atmosphere of N₂ were added N-methylmorpholine (3.34 g, 33.0 mmol) and(S)-3-(3-bromo-5-(difluoromethyl)phenyl)-2-((tert-butoxycarbonyl) amino) propanoic acid (650 mg, 1.7 mmol) and HOBt (45 mg, 0.33 mmol) and EDCI (632 mg, 3.3 mmol). The mixture was warmed to room temperature and stirred for 16 h, then diluted with DCM (100 mL) and H₂O. The organic and aqueous layer was separated and the aqueous layer was extracted with DCM (100 ml×3). The combined organic layers were dried over anhydrous Na₂SO₄ and filtered. The filtrate was concentrated under reduced pressure and the residue was purified by silica gel column chromatography to give methyl(S)-1-((S)-3-(3-bromo-5-(difluoromethyl)phenyl)-2-((tert-butoxycarbonyl) amino) propanoyl) hexahydropyridazine-3-carboxylate (510 mg, 56% yield) as a solid. LCMS (ESI): m/z [M+H] calc'd for C₂₁H₂₈BrF₂N₃O₅ 519.1; found 520.3.

Step 5. To a mixture of 4,4,5,5-tetramethyl-2-(tetramethyl-1,3,2-dioxaborolan-2-yl)-1,3,2-dioxaborolane (488 mg, 1.92 mmol) and methyl(S)-1-((S)-3-(3-bromo-5-(difluoromethyl)phenyl)-2-((tert-butoxycarbonyl) amino) propanoyl) hexahydropyridazine-3-carboxylate (500 mg, 0.96 mmol) in 1,4-dioxane (5 mL) was added Pd(dppf)Cl₂ (70 mg, 0.07 mmol) and KOAc (236 mg, 2.4 mmol) in portions. The mixture was heated to 90° C. and stirred for 4 h then diluted with H₂O (100 mL). The mixture was extracted with DCM (100 mL×3) and the combined organic layers were dried over anhydrous Na₂SO₄ and filtered. The filtrate was concentrated under reduced pressure and the residue was purified by silica gel column chromatography to give methyl(S)-1-((S)-2-((tert-butoxycarbonyl) amino)-3-(3-(difluoromethyl)-5-(4,4,5,5-tetramethyl-1,3,2-dioxaborolan-2-yl)phenyl) propanoyl) hexahydropyridazine-3-carboxylate (423 mg, 73% yield) as a solid. LCMS (ESI): m/z [M+H] calc'd for C₂₇H₄₀BF₂N₃O₇ 567.3; found 568.2.

Step 6. To a mixture of methyl(S)-1-((S)-2-((tert-butoxycarbonyl) amino)-3-(3-(difluoromethyl)-5-(4,4,5,5-tetramethyl-1,3,2-dioxaborolan-2-yl)phenyl) propanoyl) hexahydropyridazine-3-carboxylate (260 mg, 0.47 mmol), (S)-3-(5-bromo-1-ethyl-2-(2-(1-methoxyethyl) pyridin-3-yl)-1H-indol-3-yl)-2,2-dimethylpropan-1-ol and Pd(dppf)Cl₂ (34 mg, 0.05 mmol) in 1,4-dioxane (3 mL) and H₂O (0.6 mL) was added K₂CO₃ (163 mg, 1.12 mmol). The mixture was heated to 60° C. and stirred for 16 h, then diluted with H₂O (100 mL) and extracted with DCM (100 mL×3). The combined organic layers were dried over anhydrous Na₂SO₄ and filtered. The filtrate was concentrated under reduced pressure and the residue was purified by silica gel column chromatography to give methyl(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylate (350 mg, 78% yield) as a solid. LCMS (ESI): m/z [M+H] calc'd for C₄₄H₅₇F₂N₅O₇ 805.4; found 806.6.

Step 7. To a mixture of methyl(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylate (350 mg, 0.43 mmol) in THF (2.8 mL) at 0° C. was added LiOH H₂O (54 mg, 1.3 mmol) in H₂O (0.7 mL). The mixture was warmed to room temperature and stirred for 2 h, then acidified to pH 5 with 1M HCl and extracted with EtOAc (50 mL×3). The combined organic layers were dried over anhydrous Na₂SO₄, filtered and the filtrate was concentrated under reduced pressure to give (S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylic acid (356 mg) was used directly in the next step without further purification. LCMS (ESI): m/z [M+H] calc'd for C₄₃H₅₅F₂N₅O₇ 791.4; found 792.6.

Step 8. To a mixture of S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylic acid (356 mg, 0.45 mmol) and DIPEA (1.74 g, 13.5 mmol) in DCM were added EDCI (2.41 g, 12.6 mmol) and HOBt (304 mg, 2.3 mmol). The mixture was stirred for 16 h then H₂O was added and the mixture extracted with EtOAc (200 mL×3). The combined organic layers were washed with brine (50 mL×4), dried over anhydrous Na₂SO₄ and filtered. The filtrate was concentrated under reduced pressure and the residue was purified by silica gel column chromatography to give tert-butyl ((6³S,4S)-25-(difluoromethyl)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (202 mg, 51% yield). LCMS (ESI): m/z [M+H] calc'd for C₄₃H₅₃F₂N₅O₆ 773.4; found 774.6.

Step 9. To a mixture of tert-butyl ((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (202 mg, 0.26 mmol) in DCM (2 mL) at 0° C. was added TFA (1.0 mL) dropwise. The mixture was stirred at 0° C. for 1.5 h, then concentrated under reduced pressure and dried azeotropically with toluene (3 mL×3) to give (6³S,4S)-4-amino-25-(difluoromethyl)-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione, which was used directly in the next without further purification. LCMS (ESI): m/z [M+H] calc'd for C₃₈H₄₅F₂N₅O₄ 673.3; found 674.5.

Step 10. To a mixture of (6³S,4S)-4-amino-25-(difluoromethyl)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (202 mg, 0.3 mmol) and (2S)-2-[(tert-butoxycarbonyl) (methyl) amino]-3-methylbutanoic acid (139 mg, 0.6 mmol) in THF under an atmosphere of Ar were added DIPEA (581 mg, 4.5 mmol), EDCI (86 mg, 0.45 mmol) and HOBt (61 mg, 0.45 mmol). The mixture was stirred for 16 h, then H₂O (100 mL) added and the mixture extracted with EtOAc (200 mL×3). The combined organic layers were washed with brine (50 mL×3), dried over anhydrous Na₂SO₄ and filtered. The filtrate was concentrated under reduced pressure and the residue was purified by silica gel column chromatography to give tert-butyl ((2S)-1-(((6³S,4S)-25-(difluoromethyl)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (135 mg, 46% yield) as a solid. LCMS (ESI): m/z [M+H] calc'd for C₄₉H₆₄F₂N₆O₇ 886.5; found 887.6.

Step 11. To a mixture of tert-butyl ((2S)-1-(((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (130 mg, 0.15 mmol) in DCM at 0° C. under an atmosphere of N₂ was added TFA (1.0 mL) dropwise. The mixture was stirred at 0° C. for 1.5 h, then concentrated under reduced pressure and dried azeotropically with toluene (3 mL×3) to give (2S)—N-((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (130 mg), which was used directly in the next step without further purification. LCMS (ESI): m/z [M+H] calc'd for C₄₄H₅₆F₂N₆O₅ 786.4; found 787.6.

Step 12. To a mixture of (2S)—N-((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (130 mg, 0.17 mmol) and (3S)-1-(prop-2-enoyl) pyrrolidine-3-carboxylic acid (56 mg, 0.33 mmol) in MeCN (1.5 mL) at 0° C. under an atmosphere of N₂ were added DIPEA (427 mg, 3.3 mmol) and CIP (69 mg, 0.25 mmol). The mixture was stirred at 0° C. for 1 h, then H₂O (100 mL) was added and the mixture extracted with EtOAc (200 mL×3). The combined organic layers were washed with brine (50 mL×3), dried over anhydrous Na₂SO₄ and filtered. The filtrate was concentrated under reduced pressure and the residue was purified by preparative-HPLC to give (3S)-1-acryloyl-N-((2S)-1-(((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide (58 mg, 36% yield) as a solid. LCMS (ESI): m/z [M+H] calc'd for C₅₂H₆₅F₂N₇O₇ 937.4; found 938.1; ¹H NMR (400 MHZ, DMSO-d₆) δ 8.78 (dd, J=4.8, 1.7 Hz, 1H), 8.43-8.21 (m, 1H), 8.02 (s, 2H), 7.93-7.81 (m, 2H), 7.76 (dd, J=9.3, 3.9 Hz, 1H), 7.66 (d, J=8.7 Hz, 1H), 7.56 (dd, J=7.7, 4.8 Hz, 1H), 7.34 (d, J=5.4 Hz, 1H), 7.20-6.86 (m, 1H), 6.80-6.40 (m, 1H), 6.15 (ddt, J=16.8, 4.9, 2.4 Hz, 1H), 5.90-5.60 (m, 1H), 5.59-5.19 (m, 2H), 4.71 (dd, J=10.7, 3.1 Hz, 1H), 4.40-4.17 (m, 3H), 4.12-3.90 (m, 3H), 3.85-3.71 (m, 1H), 3.61 (tdd, J=23.4, 9.9, 4.3 Hz, 6H), 3.40-3.30 (m, 2H), 3.11 (d, J=6.8 Hz, 3H), 3.08-2.90 (m, 2H), 2.87 (s, 2H), 2.84 (s, 3H), 2.69-2.30 (d, J=16.5 Hz, 1H), 2.30-1.79 (m, 5H), 1.75-1.45 (m, 2H), 1.40 (d, J=6.1 Hz, 3H), 1.05-0.85 (m, 6H), 0.85-0.66 (m, 6H), 0.57 (d, J=11.8 Hz, 3H).

### Example A741. Synthesis of (3S)-1-acryloyl-N-((2S)-1-(((23S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-piperidinacycloundecaphane-4-yl) amino)-3-methyl-1-oxobutan-2-yl)-N-methylpyrrolidine-3-carboxamide

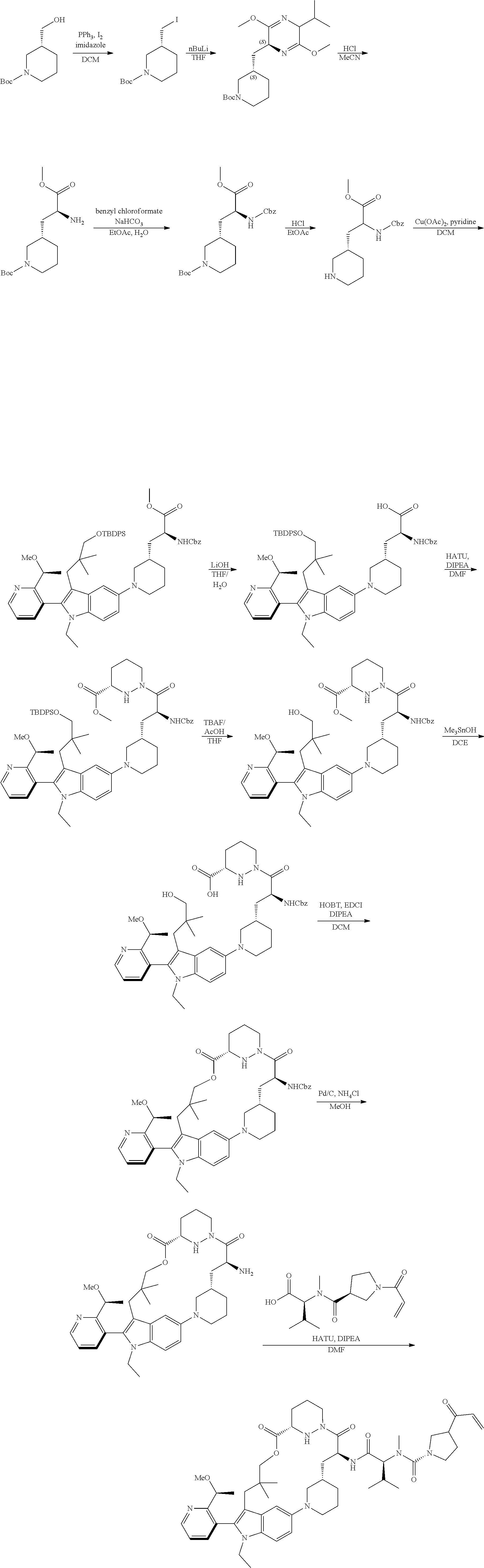

Step 1. A solution of tert-butyl (3R)-3-(hydroxymethyl) piperidine-1-carboxylate (10 g, 46.45 mmol) in DCM (200 mL) at 0° C., was added PPh₃ (15.8 g, 60.4 mmol), Imidazole (4.7 g, 69.7 mmol) and |2 (14.1 g, 55.74 mmol). The reaction suspension was stirred at 20° C. for 17 h, then concentrated under reduced pressure. The residue was purified by silica gel column chromatography to afford tert-butyl (3R)-3-(iodomethyl) piperidine-1-carboxylate (10 g, 66% yield) as oil. LCMS (ESI): m/z [M+H] calc'd for C₁₁H₂₀INO₂ 325.1; found no mass.

Step 2. To a mixture of 3-isopropyl-2,5-dimethoxy-3,6-dihydropyrazine (10.8 g, 58.9 mmol) in THF (150 mL) at −60° C. under an atmosphere of N₂ was added n-BuLi (47 mL, 2.5 M in hexane, 117.7 mmol) dropwise. The mixture was warmed to 0° C. and was stirred for 2 h, then re-cooled to-60° C., and a solution of tert-butyl (3R)-3-(iodomethyl) piperidin-1-yl formate (9.60 g, 29.4 mmol) in THF (50 mL) was slowly added dropwise. The mixture was stirred at −60° C. for 2 h then warmed to room temperature and stirred for 2 h. Saturated NH₄Cl (150 mL) was slowly added and the mixture extracted with EtOAc (150 mL×2). The combined organic layers were washed with brine (200 mL), dried over anhydrous Na₂SO₄ and filtered. The filtrate was reduced under reduced pressure and the residue was purified by silica gel column chromatography to give tert-butyl (3S)-3-{[(2S)-5-isopropyl-3,6-dimethoxy-2,5-dihydropyrazin-2-yl]methyl}piperidin-1-yl formate (5.3 g, 46% yield) as a gum. LCMS (ESI): m/z [M+H] calc'd for C₂₀H₃₅N₃O₄ 381.5; found 382.3.

Step 3. A mixture of tert-butyl (3S)-3-{[(2S)-5-isopropyl-3,6-dimethoxy-2,5-dihydropyrazin-2-yl]methyl}piperidin-1-yl formate (5.30 g, 13.9 mol) in MeCN (4 mL) was added 1M HCl (27.7 mL, 27.7 mmol) dropwise. The mixture was stirred for 2 h, then saturated NaHCO₃until ˜pH 7-8, then extracted with DCM (30 mL×2). The combined organic layers was dried over anhydrous Na₂SO₄, filtered and concentrated under reduced pressure to give methyl(S)-tert-butyl 3-((S)-2-amino-3-methoxy-3-oxopropyl) piperidine-1-carboxylate (4.3 g, 95% yield) as an oil, which was used in next step without further purification. LCMS (ESI): m/z [M+H] calc'd for C₁₄H₂₆N₂O₄ 286.2; found 287.3.

Step 4. To a mixture of methyl(S)-tert-butyl 3-((S)-2-amino-3-methoxy-3-oxopropyl) piperidine-1-carboxylate (4.30 g, 15.0 mmol) in EtOAc (30 mL) and H₂O (20 mL) at-10° C. was added NaHCO₃ (3.77 g, 44.88 mmol). The mixture was stirred at −10° C. for 10 min, then a solution of benzyl chloroformate (3.83 g, 22.44 mmol) was added dropwise. The mixture was warmed to 0° C. and stirred for 1 h, then H₂O (50 mL) was added and the mixture extracted with EtOAc (50 mL×2). The combined organic layers were dried over anhydrous Na₂SO₄, filtered, the filtrate was concentrated under reduced pressure to give tert-butyl (3S)-3-[(2S)-2-{[(benzyloxy) carbonyl]amino}-3-methoxy-3-oxopropyl]piperidine-1-carboxylate (4.0 g, 60% yield) as a gum. LCMS (ESI): m/z [M-Boc+H] calc'd for C₁₇H₂₄N₂O₄ 320.2; found 321.3.

Step 5. To a mixture of tert-butyl (3S)-3-[(2S)-2-{[(benzyloxy) carbonyl]amino}-3-methoxy-3-oxopropyl]piperidine-1-carboxylate (1.0 g, 2.38 mmol) in EtOAc (8 mL) was added 2M HCl in EtOAc (11.9 mL, 23.8 mmol). The mixture was stirred for 2 h, then saturated NaHCO₃added until ˜pH 8-9, and the mixture extracted with DCM (30 mL×3). The combined organic layer was dried over anhydrous Na₂SO₄, filtered and concentrated under reduced pressure to give (2S)-2-{[(benzyloxy) carbonyl]amino}-3-[(3S)-piperidin-3-yl]propanoate (740 mg, 91% yield) as a gum. LCMS (ESI): m/z [M+H] calc'd for C₁₇H₂₄N₂O₄ 320.2; found 321.2.

Step 6. To a mixture of (3-{3-[(tert-butyldiphenylsilyl)oxy]-2,2-dimethylpropyl}-1-ethyl-2-{2-[(1S)-1-methoxyethyl]pyridin-3-yl}indol-5-yl) boranediol (5.47 g, 8.43 mmol) and methyl (2S)-2-{[(benzyloxy) carbonyl]amino}-3-[(3S)-piperidin-3-yl]propanoate (2.70 g, 8.43 mmol) in DCM (70 mL) was added Cu (OAc) 2 (6.06 g, 16.86 mmol) and pyridine (2.0 g, 25.3 mmol). The mixture was stirred under an atmosphere of O₂ for 48 h, then diluted with DCM (200 mL) and washed with H₂O (150 mL×2). The organic layer was dried over anhydrous Na₂SO₄, filtered, concentrated under reduced pressure and the residue was purified by silica gel column chromatography to give methyl 2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoate (3.6 g, 42% yield) as a solid. LCMS (ESI): m/z [M/2+H] calc'd for C₅₆H₇₀N₄O₆Si 462.3; found 462.3.

Step 7. To a mixture of methyl 2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoate (3.60 g, 3.57 mmol) in THF (60 mL) and H₂O (30 mL) was added LiOH (342 mg, 14.28 mmol). The mixture was stirred for 2 h, then diluted with H₂O (150 mL), then 1M HCl was added slowly until ˜pH 3-4 and the mixture extracted with EtOAc (200 ml×3). The combined organic layers were dried over anhydrous Na₂SO₄, filtered and the filtrate concentrated under reduced pressure to give 2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoic acid (3.3 g, 85% yield) as a solid, which was used directly in the next step without further purification. LCMS (ESI): m/z [M/2+H] calc'd for C₅₅H₆₈N₄O₆Si 455.3; found 455.3.

Step 8. To a mixture of methyl 2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoate (3.30 g, 2.91 mmol) in DMF (40 mL) was added methyl (3S)-1,2-diazinane-3-carboxylate (0.42 g, 2.91 mmol), HATU (2.21 g, 5.82 mmol) and DIPEA (2.26 g, 17.46 mmol). The mixture was stirred for 3 h, then poured into ice-H₂O and extracted with EtOAc (120 mL×2). The combined organic layers were washed with saturated NaHCO₃ (150 mL), brine (150 mL), dried over anhydrous Na₂SO₄ and filtered. The filtrate was concentrated under reduced pressure and the residue was purified by silica gel column chromatography to give methyl (3S)-1-[(2S)-2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoyl]-1,2-diazinane-3-carboxylate (2.9 g, 95% yield) as a gum. LCMS (ESI): m/z [M/2+H] calc'd for C₆₁H₇₈N₆O₇Si 518.3; found 518.3.

Step 9. To a mixture of methyl (3S)-1-[(2S)-2-{[(benzyloxy) carbonyl]amino}-3-[(3S)-1-(3-{3-[(tert-butyldiphenylsilyl)oxy]-2,2-dimethylpropyl}-1-ethyl-2-{2-[(1S)-1-methoxyethyl]pyridin-3-yl}indol-5-yl) piperidin-3-yl]propanoyl]-1,2-diazinane-3-carboxylate (1.70 g, 1.64 mmol) was added a mixture of 1M TBAF in THF (19.68 mL, 19.68 mmol) and AcOH (1.18 g, 19.68 mmol). The reaction was heated to 60° C. and stirred for 22 h, then diluted with EtOAc (80 mL) and washed with saturated NaHCO₃ (80 mL), H₂O (60 mL×2) and brine (60 mL). The organic layer was dried over anhydrous Na₂SO₄, filtered, the filtrate was concentrated under reduced pressure and the residue was purified by preparative-HPLC to give methyl (3S)-1-[(2S)-2-{[(benzyloxy) carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylate (1.0 g, 73% yield) as a solid. LCMS (ESI): m/z [M/2+H] calc'd for C₄₅H₆₀N₆O₇ 399.2; found 399.4.

Step 10. To a mixture m methyl (3S)-1-[(2S)-2-{[(benzyloxy) carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylate (1.0 g, 1.1 mmol) in 1,2-dichloroethane (10 mL) was added Me₃SnOH (1.42 g 7.84 mmol). The mixture was heated to 65° C. and stirred for 10 h, then filtered and the filtrate was concentrated under reduced pressure to give (3S)-1-[(2S)-2-{[(benzyloxy) carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylic acid (1.0 g, 99% yield) as a gum. The product was used in the next step without further purification. LCMS (ESI): m/z [M/2+H] calc'd for C₄₄H₅₈N₆O₇ 392.2; found 392.3.

Step 11. To a mixture of (3S)-1-[(2S)-2-{[(benzyloxy) carbonyl]amino}-3-[(3S)-1-[1-ethyl-3-(3-hydroxy-2,2-dimethylpropyl)-2-{2-[(1S)-1-methoxyethyl]pyridin-3-yl}indol-5-yl]piperidin-3-yl]propanoyl]-1,2-diazinane-3-carboxylic acid (1.0 g, 1.1 mmol) in DCM (30 mL) at 0° C. was added HOBT (1.51 g, 11.2 mmol), N-(3-dimethylaminopropyl)-N′-ethylcarbodiimide HCl (6.44 g, 33.6 mmol) and DIPEA (5.79 g, 44.8 mmol). The mixture was warmed to room temperature and stirred for 6 h, then diluted with H₂O and extracted with EtOAc (100 mL×3). The combined organic layers were dried over anhydrous Na₂SO₄, filtered, the filtrate was concentrated under reduced pressure and the residue was purified by silica gel column chromatography to give benzyl N-[(6S,8S,14S)-22-ethyl-21-{2-[(1S)-1-methoxyethyl]pyridin-3-yl}-18, 18-dimethyl-9, 15-dioxo-16-oxa-2, 10,22,28-tetraazapentacyclo[18.5.2.1\hat{ }{2,6}0.1\hat{ }{10, 14}0.0\hat{ }{23,27}]nonacosa-1 (26),20,23 (27),24-tetraen-8-yl]carbamate (340 mg, 36% yield) as a solid. LCMS (ESI): m/z [M+H] calc'd for C₄₄H₅₆N₆O₆ 383.2; found 383.3.

Step 12. A mixture of benzyl N-[(6S,8S, 14S)-22-ethyl-21-{2-[(1S)-1-methoxyethyl]pyridin-3-yl}-18, 18-dimethyl-9,15-dioxo-16-oxa-2, 10,22,28-tetraazapentacyclo[18.5.2.1\hat{ }{2,6}0.1\hat{ }{10, 14}0.0\hat{ }{23,27}]nonacosa-1 (26),20,23 (27), 24-tetraen-8-yl]carbamate (250 mg, 0.33 mmol), Pd/C (100 mg) and NH₄Cl (353 mg, 6.6 mmol) in MeOH (5 mL) was stirred under an atmosphere of H₂ for 4 h. The mixture was filtered through Celite and the filtrate was concentrated under reduced pressure. The residue was dissolved in DCM (30 mL) and washed with saturated NaHCO₃ (20 mL), H₂O (20 mL) and brine (20 mL). The organic layer was dried over Na₂SO₄, filtered, and the filtrate concentrated under reduced pressure to give (6S,8S,14S)-8-amino-22-ethyl-21-{2-[(1S)-1-methoxyethyl]pyridin-3-yl}-18, 18-dimethyl-16-oxa-2, 10,22,28-tetraazapentacyclo[18.5.2.1\hat{ }{2,6}0.1\hat{ }{10,14}0.0\hat{ }{23,27}]nonacosa-1 (26),20,23 (27),24-tetraene-9,15-dione, which was used in next step without further purification. LCMS (ESI): m/z [M+H] calc'd for C₃₆H₅₀N₆O₄ 631.4; found 631.4.

Step 13. To a mixture of (6S,8S,14S)-8-amino-22-ethyl-21-{2-[(1S)-1-methoxyethyl]pyridin-3-yl}-18,18-dimethyl-16-oxa-2,10,22,28-tetraazapentacyclo[18.5.2.1\hat{ }{2,6}. 1\hat{ }{10,14}0.0\hat{ }{23,27}]nonacosa-1 (26),20,23 (27),24-tetraene-9,15-dione (300 mg, 0.48 mmol), (2S)-3-methyl-2-{N-methyl-1-[(3S)-1-(prop-2-enoyl) pyrrolidin-3-yl]formamido}butanoic acid (136 mg, 0.48 mmol) and DIPEA (620 mg, 4.8 mmol) in DMF (5 mL) at 0° C. was added HATU (183 mg, 0.48 mmol). The mixture was stirred at 0-5° C. for 1 h, then diluted with EtOAc (50 mL), washed with H₂O (50 mL×2), brine (50 mL), dried over Na₂SO₄ and filtered. The filtrate was concentrated under reduced pressure and the residue was purified by silica gel column chromatography to give (2S)—N-[(6S,8S, 14S)-22-ethyl-21-{2-[(1S)-1-methoxyethyl]pyridin-3-yl}-18,18-dimethyl-9,15-dioxo-16-oxa-2,10,22,28-tetraazapentacyclo[18.5.2.1\hat{ }{2,6}0.1\hat{ }{10,14}0.0\hat{ }{23,27}]nonacosa-1 (26),20,23 (27),24-tetraen-8-yl]-3-methyl-2-{N-methyl-1-[(3S)-1-(prop-2-enoyl) pyrrolidin-3-yl]formamido}butanamide (90 mg, 20% yield) as a solid. LCMS (ESI): m/z [M+H] calc'd for C₅₀H₇₀N₈O₇ 895.5; found 895.4; ¹H NMR (400 MHz, CD₃OD) δ 8.71 (dd, J=4.8, 1.5 Hz, 1H), 7.86 (dd, J=7.7, 1.5 Hz, 1H), 7.51 (dd, J=7.7, 4.8 Hz, 1H), 7.36 (dd, J=8.9, 1.9 Hz, 1H), 7.22 (d, J=12.1 Hz, 1H), 7.09 (dd, J=8.9, 1.9 Hz, 1H), 6.59 (dt, J=16.9, 9.9 Hz, 1H), 6.26 (ddd, J=16.8, 5.0, 1.9 Hz, 1H), 5.80-5.67 (m, 1H), 5.59-5.46 (m, 1H), 4.93 (d, J=12.4 Hz, 1H), 4.66 (dd, J=11.1, 6.4 Hz, 1H), 4.45 (d, J=12.6 Hz, 1H), 4.28-4.19 (m, 1H), 4.13 (dd, J=14.5, 7.2 Hz, 1H), 4.02-3.87 (m, 1H), 3.87-3.36 (m, 11H), 3.16 (s, 2H), 3.10 (d, J=3.4 Hz, 2H), 2.76 (dd, J=26.9, 13.5 Hz, 3H), 2.61 (s, 1H), 2.35-1.97 (m, 5H), 1.78 (dd, J=25.4, 22.1 Hz, 10H), 1.45 (d, J=6.2 Hz, 3H), 1.04 (d, J=6.2 Hz, 3H), 0.95 (dd, J=6.5, 1.8 Hz, 3H), 0.83 (d, J=6.6 Hz, 3H), 0.72 (d, J=31.8 Hz, 6H).

### Example A715. Synthesis of benzyl ((23S,6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-piperidinacycloundecaphane-4-yl) carbamate

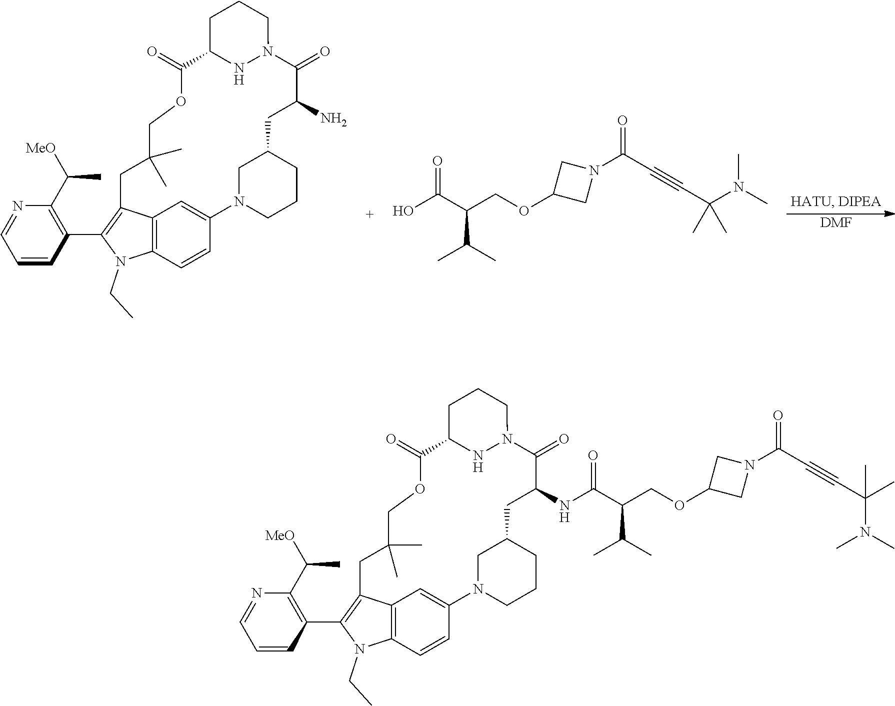

To a solution of ((23S,6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-piperidinacycloundecaphane-5,7-dione (50 mg, 0.08 mmol), (R)-2-(((1-(4-(dimethylamino)-4-methylpent-2-ynoyl) azetidin-3-yl)oxy) methyl)-3-methylbutanoic acid (26 mg, 0.08 mmol) and DIPEA (31 mg, 0.24 mmol) in DMF (1 mL) at 0° C., was added HATU (30 mg, 0.08 mmol). The reaction mixture was stirred at 0-5° C. for 1 h, then diluted with EtOAc (20 mL), washed with H₂O (20 mL×2) and brine (20 mL). The organic phase was separated and dried over anhydrous sodium sulfate, filtered and concentrated under reduced pressure. The residue was purified by silica gel column chromatography to give (2R)-2-(((1-(4-(dimethylamino)-4-methylpent-2-ynoyl) azetidin-3-yl)oxy) methyl)-N-((23S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-piperidinacycloundecaphane-4-yl)-3-methylbutanamide as solid. ¹H NMR (400 MHZ, CD₃OD) δ 8.71 (d, J=4.7 Hz, 1H), 8.24 (s, 1H), 8.10 (dd, J=26.7, 7.8 Hz, 1H), 7.86 (d, J=7.7 Hz, 1H), 7.51 (dd, J=7.7, 4.8 Hz, 1H), 7.39 (dd, J=8.9, 3.1 Hz, 1H), 7.26 (d, J=16.6 Hz, 1H), 7.11 (d, J=8.8 Hz, 1H), 5.61 (s, 1H), 4.50-4.28 (m, 3H), 4.27-4.07 (m, 3H), 3.98 (ddd, J=25.6, 13.4, 5.1 Hz, 2H), 3.84-3.72 (m, 2H), 3.62 (dd, J=10.7, 4.8 Hz, 2H), 3.55 (d, J=7.1 Hz, 2H), 3.47 (d, J=6.5 Hz, 2H), 3.16 (s, 3H), 3.03-2.91 (m, 1H), 2.76 (dd, J=28.7, 15.2 Hz, 3H), 2.62 (s, 1H), 2.40 (t, J=7.0 Hz, 3H), 2.33 (dd, J=14.3, 5.0 Hz, 4H), 2.05 (d, J=11.6 Hz, 1H), 1.99-1.64 (m, 10H), 1.64-1.55 (m, 1H), 1.51-1.42 (m, 6H), 1.37 (d, J=12.3 Hz, 3H), 1.05 (s, 3H), 0.94 (ddd, J=9.3, 6.7, 2.0 Hz, 6H), 0.76 (d, J=3.8 Hz, 3H), 0.69 (s, 3H). LCMS (ESI): m/z [M+H] calc'd for C₅₃H₇₆N₈O₇ 936.6; found 937.4.

### Example A347. Synthesis of (2S)—N-[(7S,13S)-21-ethyl-20-{2-[(1S)-1-methoxyethyl]pyridin-3-yl}-17,17-dimethyl-8,14-dioxo-15-oxa-4-thia-9,21,25,27,28-pentaazapentacyclo[17.5.2.1\hat{ }{2,5}0.1\hat{ }{9,13}0.0\hat{ }{22,26}]octacosa-1 (25),2,5 (28), 19,22 (26),23-hexaen-7-yl]-3-methyl-2-{N-methyl-1-[(3S)-1-(prop-2-enoyl) pyrrolidin-3-yl]formamido}butanamide

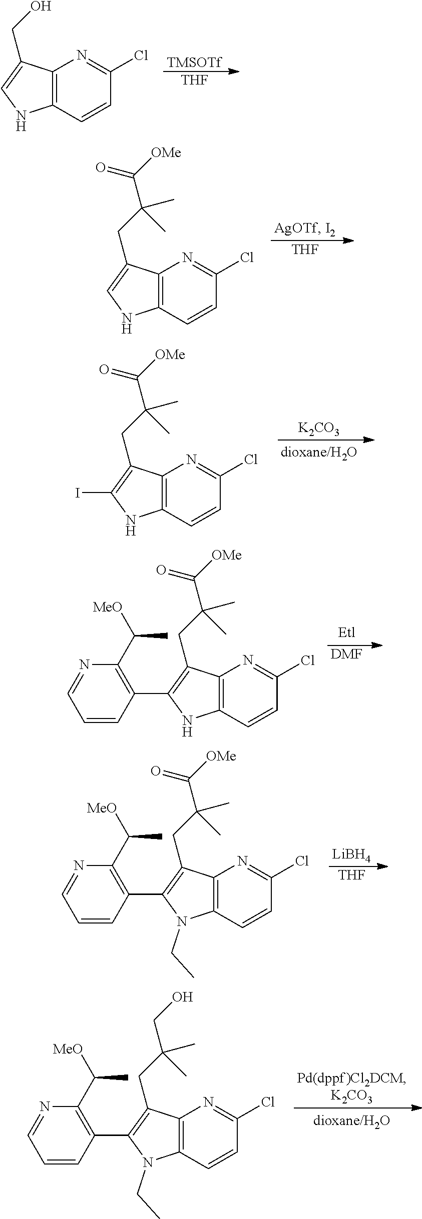

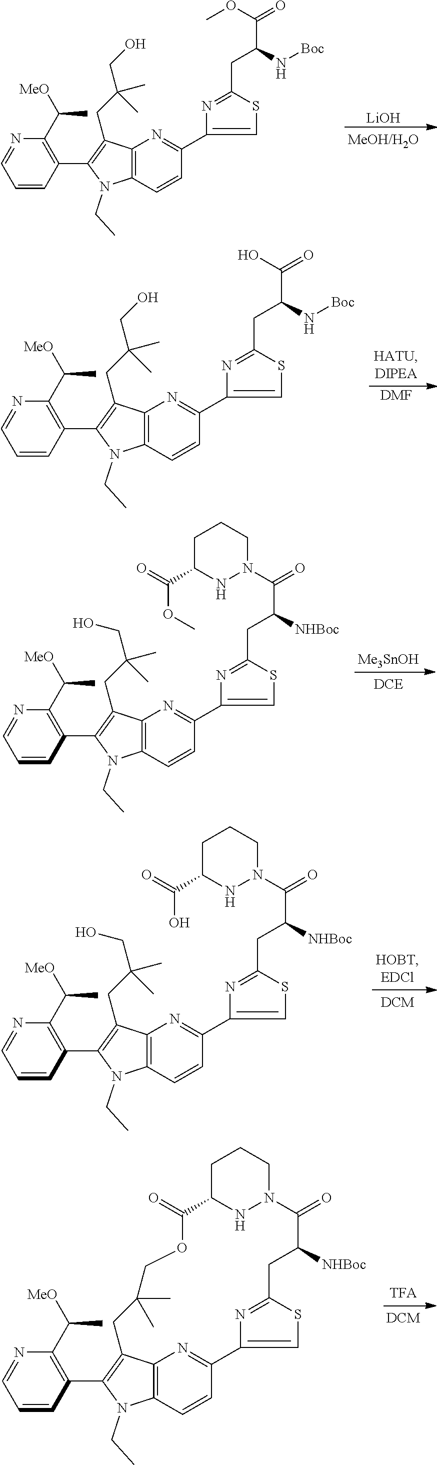

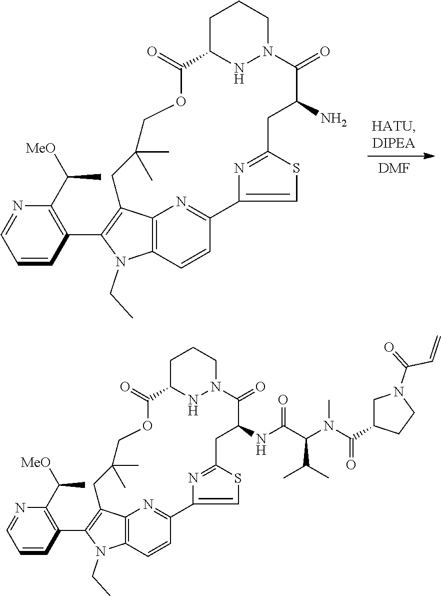

Step 1. To a mixture of (5-chloro-1H-pyrrolo [3,2-b]pyridin-3-yl) methanol (3.5 g, 19 mmol), and ((1-methoxy-2-methylprop-1-en-1-yl)oxy) trimethylsilane (6.7 g, 38 mmol) in THF (50 ml) at 0° C. was added TMSOTf (3.8 g, 17 mmol) dropwise. The mixture was stirred at 0-5° C. for 2 h, then diluted with EtOAc (100 mL) and washed with saturated NaHCO₃ (50 mL) and brine (50 mL×2). The organic layer was dried over Na₂SO₄, filtered and the filtrate was concentrated under reduced pressure. The residue was purified by silica gel column chromatography to give methyl 3-(5-chloro-1H-pyrrolo [3,2-b]pyridin-3-yl)-2,2-dimethylpropanoate (3.0 g, 59% yield) as a solid. LCMS (ESI): m/z [M+H] calc'd for C₁₃H₁₅ClN₂O₂ 266.1; found 267.1.

Step 2. To a mixture of methyl 3-(5-chloro-1H-pyrrolo [3,2-b]pyridin-3-yl)-2,2-dimethylpropanoate (3.0 g, 11 mmol) in anhydrous THF (50 mL) at 0° C. was added AgOTf (4.3 g, 17 mmol) and 12 (2.9 g, 11 mmol). The mixture was stirred at 0° C. for 2 h, then saturated Na₂SO₃ (20 mL) and EtOAc (50 mL) added. The mixture was filtered and the filtrate was washed with brine (50 mL), dried over Na₂SO₄ and filtered. The filtrate was concentrated under reduced pressure and the residue was purified by silica gel column chromatography to give methyl 3-(5-chloro-2-iodo-1H-pyrrolo [3,2-b]pyridin-3-yl)-2,2-dimethylpropanoate (2.3 g, 52% yield) as a solid. LCMS (ESI): m/z [M+H] calc'd for C₁₃H₁₅ClIN₂O₂ 392.0; found 393.0.

Step 3. To a mixture of methyl 3-(5-chloro-2-iodo-1H-pyrrolo [3,2-b]pyridin-3-yl)-2,2-dimethylpropanoate (2.3 g, 5.9 mmol) and 2-(2-(2-methoxyethyl)phenyl)-4,4,5,5-tetramethyl-1,3,2-dioxaborolane (1.6 g, 7.1 mmol) and K₂CO₃ (2.4 g, 18 mol) in 1,4-dioxane (25 mL) and H₂O (5 mL) under an atmosphere of N₂ was added Pd(dppf)Cl₂.DCM (480 mg, 0.59 mmol). The mixture was heated to 70° C. and for 4 h, then diluted with EtOAc (200 mL) and washed with brine (25 mL), dried over Na₂SO₄ and filtered. The filtrate was concentrated under reduced pressure and the residue was purified by silica gel column chromatography to give methyl(S)-3-(5-chloro-2-(2-(1-methoxyethyl) pyridin-3-yl)-1H-pyrrolo [3,2-b]pyridin-3-yl)-2,2-dimethylpropanoate (2.0 g, 84% yield) as a solid. LCMS (ESI): m/z [M+H] calc'd for C₂₁H₂₄ClN₃O₃ 401.2; found 402.2.

Step 4. A mixture of ethyl 3-(5-chloro-2-(2-((S)-1-methoxyethyl) pyridin-3-yl)-1H-pyrrolo [3,2-b]pyridin-3-yl)-2-methylpropanoate (2.0 g, 5.0 mmol), Cs₂CO₃ (3.3 g, 10 mmol) and EtI (1.6 g, 10 mmol) in DMF (30 mL) was stirred for 10 h. The mixture was diluted with EtOAc (100 mL) and washed with brine (20 mL×4), dried over Na₂SO₄ and filtered. The filtrate was concentrated under reduced pressure and the residue was purified by silica gel column chromatography to give two diastereomers of methyl(S)-3-(5-chloro-1-ethyl-2-(2-(1-methoxyethyl) pyridin-3-yl)-1H-pyrrolo [3,2-b]pyridin-3-yl)-2,2-dimethylpropanoate (0.7 g, 32% yield; 0.6 g, 28% yield) as a solid. LCMS (ESI): m/z [M+H] calc'd for C₂₃H₂₈ClN₃O₃ 429.2; found 430.2.

Step 5. To a mixture of methyl(S)-3-(5-chloro-1-ethyl-2-(2-(1-methoxyethyl) pyridin-3-yl)-1H-pyrrolo [3,2-b]pyridin-3-yl)-2,2-dimethylpropanoate (1.9 g, 4.4 mmol) in anhydrous THF (20 mL) at 0° C. was added LiBH₄ (200 mg, 8.8 mmol). The mixture was heated to 60° C. and stirred for 4 h, then saturated NH₄Cl (20 mL) and EtOAc (50 mL) added. The aqueous and organic layers were separated and the organic layer was washed with brine (30 mL), dried over Na₂SO₄ and filtered. The filtrate was concentrated under reduced pressure and the residue was purified by silica gel column chromatography to give (S)-3-(5-chloro-1-ethyl-2-(2-(1-methoxyethyl) pyridin-3-yl)-1H-pyrrolo [3,2-b]pyridin-3-yl)-2,2-dimethylpropan-1-ol (1.5 g, 85% yield) as a solid. LCMS (ESI): m/z [M+H] calc'd for C₂₂H₂₈ClN₃O₂ 401.2; found 402.2.

Step 6. To a mixture of(S)-3-(5-chloro-1-ethyl-2-(2-(1-methoxyethyl) pyridin-3-yl)-1H-pyrrolo [3,2-b]pyridin-3-yl)-2,2-dimethylpropan-1-ol (550 mg, 1.37 mmol), (S)-(2-(2-((tert-butoxycarbonyl) amino)-3-methoxy-3-oxopropyl) thiazol-4-yl) boronic acid (907.4 mg, 2.74 mmol, 2 eq) and K₂CO₃ (568 mg, 4.11 mmol) in 1,4-dioxane (25 mL) and H₂O (5 mL) under an atmosphere of N₂ was added Pd(dppf)Cl₂·DCM (89 mg, 0.14 mmol). The mixture was heated to 70° C. and stirred for 4 h, then H₂O (50 mL) added and the mixture extracted with EtOAc (100 ml×3). The combined organic layers were washed with brine (50 mL), dried over Na₂SO₄ and filtered. The filtrate was concentrated under reduced pressure and the residue was purified by silica gel column chromatography to give methyl(S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoate (440 mg, 22% yield) as a solid, which was used directly in the next step. LCMS (ESI): m/z [M+H] calc'd for C₃₄H₄₅N₅O₆S 651.3; found 652.3.

Step 7. To a mixture of (2S)-methyl 2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoate (280 mg, 0.43 mmol) in MeOH (4 mL) was added a solution of LiOH (51 mg, 2.2 mmol) in H₂O (2 mL). The mixture was stirred for 5 h, then pH adjusted to ˜3-4 by addition of 1M HCl. The mixture was diluted with H₂O (30 mL) and extracted with EtOAc (15 mL×3). The combined organic layers were washed with brine (10 mL), dried over anhydrous Na₂SO₄, filtered and the filtrate was concentrated under reduced pressure to give (2S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoic acid (280 mg) as solid, which was used directly in the next step without further purification. LCMS (ESI): m/z [M+H] calc'd for C₃₃H₄₃N₅O₆S 637.3; found 638.3.

Step 8. To a mixture of (2S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-pyrrolo [3,2-b]pyridin-5-yl) thiazol-2-yl) propanoic acid (274 mg, 0.43 mmol) and methyl (3S)-1,2-diazinane-3-carboxylate (280 mg, 0.64 mmol) in DMF (3 mL) at 0-5° C. was added a solution of HATU (245 mg, 0.64 mmol) and DIPEA (555 mg, 4.3 mmol) in DMF (2 mL). The mixture was stirred for 1 h, then diluted with EtOAc (20 mL) and H₂O (20 mL). The aqueous and organic layers were partitioned and the organic layer was washed with H₂O (20 mL×3), brine (20 mL), dried over anhydrous Na₂SO₄ and filtered. The filtrate was concentrated under reduced pressure and the residue was purified by silica gel column chromatography to give methyl (3S)-1-[(2S)-2-{[(tert-butoxy) carbonyl]amino}-3-{4-[1-ethyl-3-(3-hydroxy-2,2-dimethylpropyl)-2-{2-[(1S)-1-methoxyethyl]pyridin-3-yl}pyrrolo [3,2-b]pyridin-5-yl]-1,3-thiazol-2-yl}propanoyl]-1,2-diazinane-3-carboxylate (230 mg, 70% yield) as a solid. LCMS (ESI): m/z [M+H] calc'd for C₃₉H₅₃NO₇S 763.4; found 764.3.

Step 9. To a mixture of methyl (3S)-1-[(2S)-2-{[(tert-butoxy) carbonyl]amino}-3-{4-[1-ethyl-3-(3-hydroxy-2,2-dimethylpropyl)-2-{2-[(1S)-1-methoxyethyl]pyridin-3-yl}pyrrolo [3,2-b]pyridin-5-yl]-1,3-thiazol-2-yl}propanoyl]-1,2-diazinane-3-carboxylate (230 mg, 0.3 mmol) in DCE (3 mL) under an atmosphere of N₂ was added Me₃SnOH (300 mg). The mixture was heated to 65° C. and stirred for 16 h, then concentrated under reduced pressure. The residue was diluted with EtOAc (20 mL), washed with H₂O (20 mL) and brine (10 mL), dried over Na₂SO₄ and filtered. The filtrate was concentrated under reduced pressure to give (3S)-1-[(2S)-2-{[(tert-butoxy) carbonyl]amino}-3-{4-[1-ethyl-3-(3-hydroxy-2,2-dimethylpropyl)-2-{2-[(1S)-1-methoxyethyl]pyridin-3-yl}pyrrolo [3,2-b]pyridin-5-yl]-1,3-thiazol-2-yl}propanoyl]-1,2-diazinane-3-carboxylic acid (200 mg) as a foam, which was used directly in the next step without further purification. LCMS (ESI): m/z [M+H] calc'd for C₃₈H₅₁N₇O₇S 749.4; found 750.3.

Step 10. To a mixture of (3S)-1-[(2S)-2-{[(tert-butoxy) carbonyl]amino}-3-{4-[1-ethyl-3-(3-hydroxy-2,2-dimethylpropyl)-2-{2-[(1S)-1-methoxyethyl]pyridin-3-yl}pyrrolo [3,2-b]pyridin-5-yl]-1,3-thiazol-2-yl}propanoyl]-1,2-diazinane-3-carboxylic acid (245 mg, 0.32 mmol) in DCM (50 mL) at 0-5° C. were added HOBT (432 mg, 3.2 mmol), EDCI HCl (1.8 g, 9.6 mmol) and DIPEA (1.65 g, 12.8 mmol). The mixture was warmed to room temperature and stirred for 16 h, then concentrated under reduced pressure. The residue was diluted with EtOAc (20 mL) and H₂O (20 mL) and the aqueous and organic layers were partitioned. The organic layer was washed with H₂O (30 mL×3), brine (30 mL), dried over anhydrous Na₂SO₄ and filtered. The filtrate was concentrated under reduced pressure and the residue was purified by preparative-TLC to give tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) carbamate (100 mg, 43% yield) as a solid. LCMS (ESI): m/z [M+H] calc'd for C₃₈H₄₉N₇O₆S 731.4; found 732.3.

Step 11. A mixture of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) carbamate (80 mg, 0.11 mmol) in DCM (0.6 mL) and TFA (0.2 mL) was stirred for 1 h. The mixture was concentrated under reduced pressure to give (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-5,7-dione (72 mg, 95% yield) as a solid. LCMS (ESI): m/z [M+H] calc'd for C₃₃H₄₁N₇O₄S 631.3; found 632.3.

Step 12. To a mixture of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-5,7-dione (120 mg, 0.39 mmol) and DIPEA (335 mg, 2.6 mmol) in DMF (1 mL) at 0° C. was added HATU (60 mg, 0.16 mmol). The mixture was stirred at 0° C. for 1 h, then diluted with H₂O (110 mL) and extracted with EtOAc (80 mL×2). The combined organic layers were washed with H₂O (100 mL), brine (100 mL), dried over anhydrous Na₂SO₄ and filtered. The filtrate was concentrated under reduced pressure and the residue was purified by preparative-TLC to give (3S)-1-acryloyl-N-((2S)-1-(((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-pyrrolo [3,2-b]pyridina-6 (1,3)-pyridazinacycloundecaphane-4-yl) amino)-3-methyl-1-oxobutan-2-yl)-N-methylpyrrolidine-3-carboxamide (1.8 mg, 2% yield) as a solid. LCMS (ESI): m/z [M+H] calc'd for C₄₇H₆₁N₉O₇S 895.4; found 896.3; ¹H NMR (400 MHZ, CD₃OD) δ 8.72 (d, J=4.5 Hz, 1H), 7.98-7.77 (m, 3H), 7.72 (dd, J=12.0, 8.6 Hz, 1H), 7.54 (dd, J=7.6, 4.8 Hz, 1H), 6.67-6.54 (m, 1H), 6.26 (m, 1H), 5.79-5.58 (m, 2H), 4.83-4.75 (m, 1H), 4.39-4.16 (m, 4H), 4.02 (dd, J=28.0, 10.6 Hz, 2H), 3.89-3.65 (m, 6H), 3.50 (m, 4H), 3.34 (d, J=6.2 Hz, 3H), 3.12 (d, J=4.0 Hz, 2H), 3.00 (s, 1H), 2.73 (m, 1H), 2.48-2.37 (m, 1H), 2.31-2.07 (m, 4H), 1.88 (d, J=11.2 Hz, 1H), 1.71 (d, J=12.8 Hz, 1H), 1.44 (m, 7H), 0.97 (dd, J=6.2, 4.4 Hz, 3H), 0.92-0.84 (m, 8H), 0.41 (d, J=6.2 Hz, 3H).

Example 647. Synthesis of 1-(4-(dimethylamino)-4-methylpent-2-ynoyl)-N-((2S)-1-(((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) amino)-3-methyl-1-oxobutan-2-yl)-4-fluoro-N-methylpiperidine-4-carboxamide

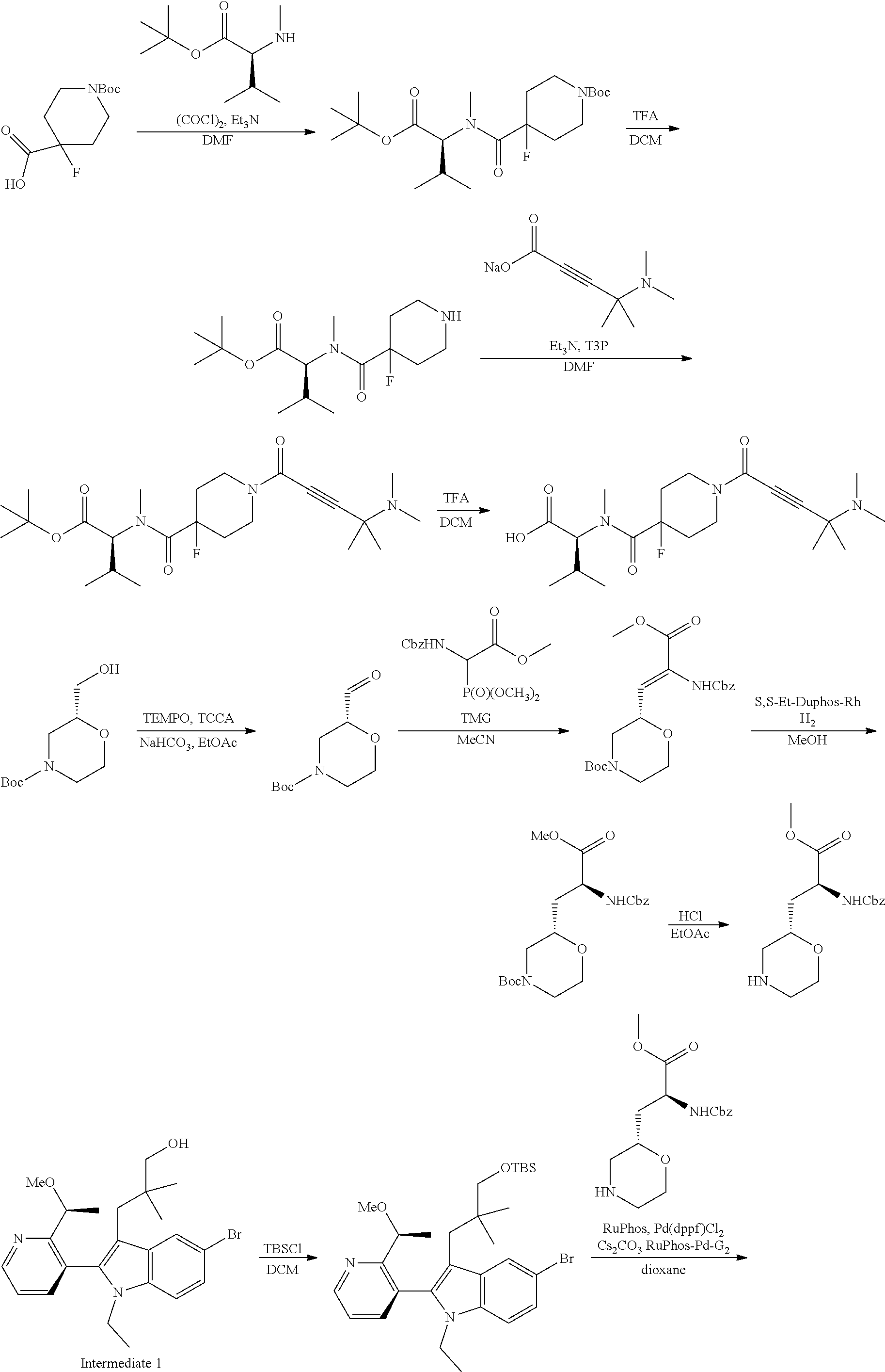

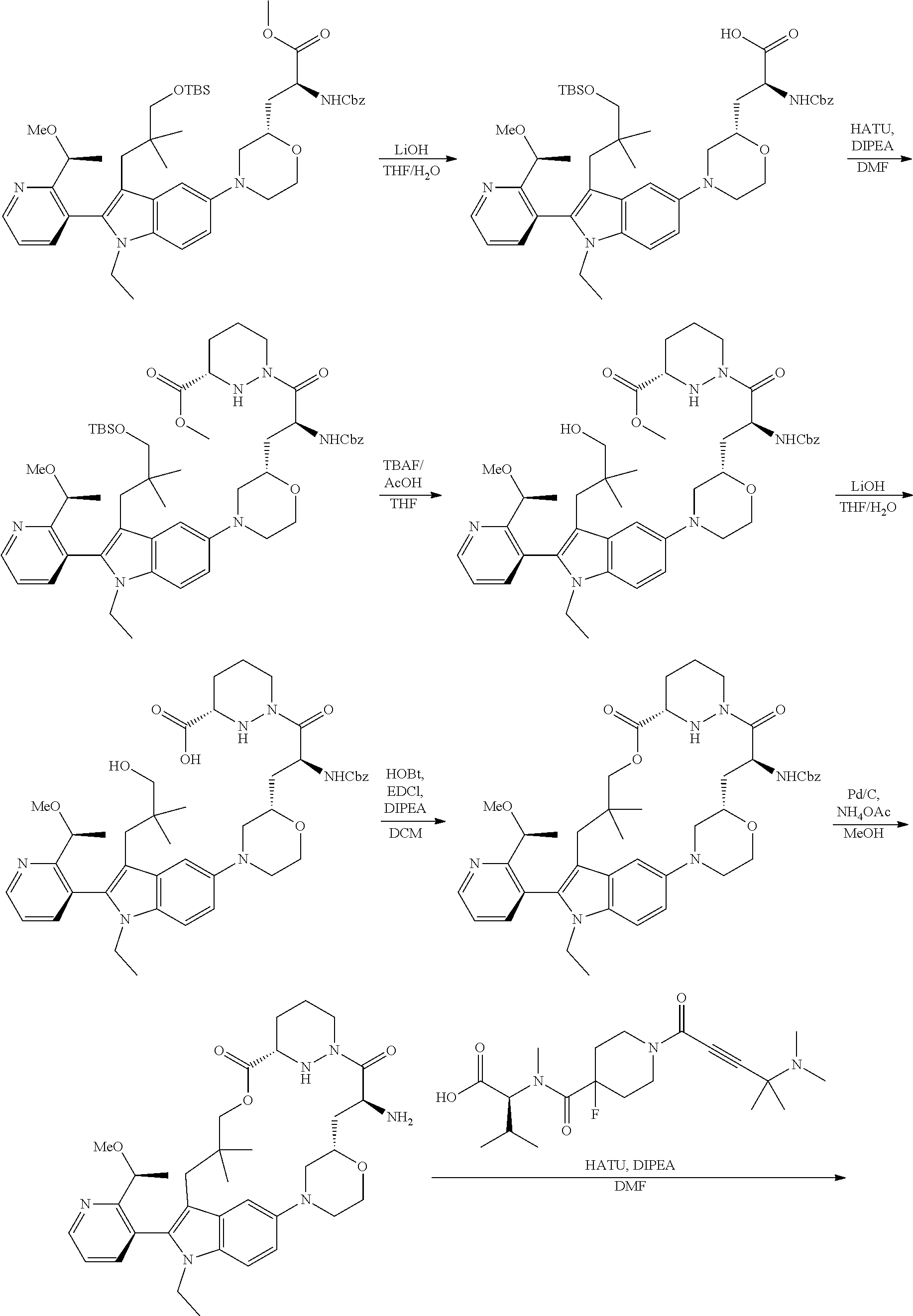

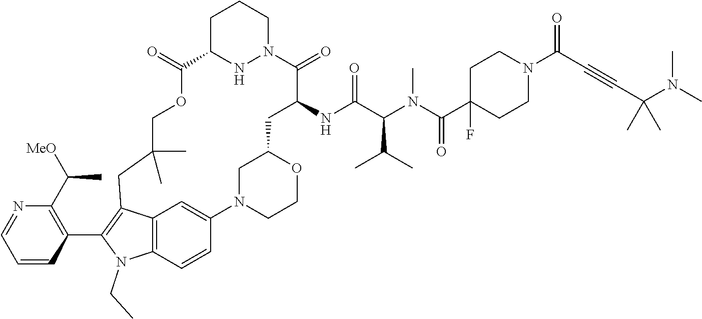

Step 1. A mixture of 1-[(tert-butoxy) carbonyl]-4-fluoropiperidine-4-carboxylic acid (2.0 g, 8.1 mmol) in DCM (20 mL) was added oxalic dichloride (1.34 g, 10.5 mmol) and DMF (30 mg, 0.4 mmol). The resulting solution was stirred at room temperature for 1 h. Et₃N (3.2 g, 3.2 mmol) and (2S)-3-methyl-2-(methylamino) butanoic acid (1.25 g, 9.5 mmol) were added and the mixture was stirred at room temperature for 1 h. H₂O (100 mL) was added and the mixture was extracted with EtOAc (50 mL×3). The combined organic layers were concentrated under reduced pressure and the residue was purified by silica gel column chromatography to give tert-butyl(S)-4-((1-(tert-butoxy)-3-methyl-1-oxobutan-2-yl) (methyl) carbamoyl)-4-fluoropiperidine-1-carboxylate (1.34 g, 45% yield) as a solid. LCMS (ESI): m/z [M+Na] calc'd for C₂₁H₃₇FN₂O₅Na 439.3; found 439.3.

Step 2. A mixture of tert-butyl(S)-4-((1-(tert-butoxy)-3-methyl-1-oxobutan-2-yl) (methyl) carbamoyl)-4-fluoropiperidine-1-carboxylate (290 mg, 0.70 mmol) in DCM (4 mL) and TFA (2 mL) was stirred at room temperature for 2 h, then concentrated under reduced pressure to give N-(4-fluoropiperidine-4-carbonyl)-N-methyl-L-valine, which was used directly in the next step without further purification. LCMS (ESI): m/z [M+H] calc'd for C₁₂H₂₁FN₂O₃ 260.2; found 261.2.

Step 3. To a solution of the tert-butyl N-(4-fluoropiperidine-4-carbonyl)-N-methyl-L-valinate (1.7 g, 5.3 mmol), sodium 4-(dimethylamino)-4-methylpent-2-ynoate (1.67 g, 9.4 mmol) and Et₃N (2.73 g, 36.9 mmol) in DMF (20 mL) stirred at 5° C. was added T3P (4.11 g, 10.7 mmol, 50 wt % in EtOAc). The reaction mixture was stirred at 5° C. for 1 h. The resulting mixture was quenched with H₂O (100 mL) and extracted with EtOAc (50 mL×3). The combined organic layers were concentrated and purified by silica gel column chromatography to give tert-butyl N-(1-(4-(dimethylamino)-4-methylpent-2-ynoyl)-4-fluoropiperidine-4-carbonyl)-N-methyl-L-valinate (1.6 g, 74.0% yield) as a solid. LCMS (ESI): m/z [M+H] calc'd for C₂₄H₄₀FN₃O₄ 453.3; found 454.2.

Step 4. To a solution of tert-butyl N-(1-(4-(dimethylamino)-4-methylpent-2-ynoyl)-4-fluoropiperidine-4-carbonyl)-N-methyl-L-valinate (50 mg, 0.11 mmol) in DCM (2 mL) was added TFA (1 mL). The reaction mixture was stirred at 20° C. for 2 h, then concentrated under reduced pressure to afford crude N-(1-(4-(dimethylamino)-4-methylpent-2-ynoyl)-4-fluoropiperidine-4-carbonyl)-N-methyl-L-valine. It was used for the next step directly without further purification. LCMS (ESI): m/z [M+H] calc'd for C₂₀H₃₂FN₃O₄ 397.2; found 398.3.

Step 5. To a solution of tert-butyl (2R)-2-(hydroxymethyl) morpholin-4-yl formate (50 g, 230 mmol) in EtOAc (1 L) was added TEMPO (715 mg, 4.6 mmol) and NaHCO₃ (58 g, 690 mmol) at 20° C. The mixture was cooled to −50° C., then TCCA (56 g, 241 mmol) in EtOAc (100 mL) was added dropwise over 30 min. The reaction mixture was warmed to 5° C. for 2 h, then quenched with 10% Na₂S203 (200 mL) and stirred for 20 min. The resulting mixture was filtered and the organic phase was separated from filtrate. The aqueous phase was extracted with EtOAc (100 ml×2). The combined organic layers were washed with H₂O (100 mL) and brine (100 mL), and dried over anhydrous Na₂SO₄. The organic layer was concentrated under reduced pressure to afford tert-butyl (2R)-2-formylmorpholin-4-yl formate (50 g, crude) as an oil.

Step 6. To a solution of tert-butyl (2R)-2-formylmorpholin-4-yl formate (49 g, 153 mmol) and methyl 2-{[(benzyloxy) carbonyl]amino}-2-(dimethoxyphosphoryl) acetate (60 g, 183 mmol) in CAN (300 mL) was added tetramethylguanidine (35 g, 306 mmol) at 0˜10° C. The reaction mixture was stirred at 10° C. for 30 min then warmed to 20° C. for 2 h. The reaction mixture was diluted with DCM (200 mL) and washed with Citric acid (10%, 200 mL) and 10% NaHCO₃ aqueous solution (200 mL). The organic phase was concentrated under reduced pressure, and purified by silica gel column chromatography to afford tert-butyl (S,Z)-2-(2-(((benzyloxy) carbonyl) amino)-3-methoxy-3-oxoprop-1-en-1-yl) morpholine-4-carboxylate (36 g, 90% yield) as solid. LCMS (ESI): m/z [M+Na] calc'd for C₂₁H₂₈N₂O₄ 420.2; found: 443.1.

Step 7. To a solution of tert-butyl (S,Z)-2-(2-(((benzyloxy) carbonyl) amino)-3-methoxy-3-oxoprop-1-en-1-yl) morpholine-4-carboxylate (49 g, 0.12 mol) in MeOH (500 mL) was added (S,S)-Et-DUPHOS-Rh (500 mg, 0.7 mmol). The mixture was stirred at 25° C. under an H₂ (60 psi) atmosphere for 48 h. The reaction was concentrated and purified by chromatography to give tert-butyl(S)-2-((S)-2-(((benzyloxy) carbonyl) amino)-3-methoxy-3-oxopropyl) morpholine-4-carboxylate (44 g, 89.8% yield) as solid. LCMS (ESI): m/z [M+Na] calc'd for C₂₁H₃₀N₂O₇ 422.2; found: 445.2.

Step 8. To a stirred solution of tert-butyl(S)-2-((S)-2-(((benzyloxy) carbonyl) amino)-3-methoxy-3-oxopropyl) morpholine-4-carboxylate (2.2 g, 5.2 mmol) in EtOAc (2 mL) was added HCl/EtOAc (25 mL) at 15° C. The reaction was stirred at 15° C. for 2 h, then concentrated under reduced pressure to afford methyl(S)-2-(((benzyloxy) carbonyl) amino)-3-((S)-morpholin-2-yl) propanoate (1.51 g, 90.4% yield) as an oil. LCMS (ESI): m/z [M+H] calc'd for C₁₆H₂₂N₂O₅ 322.1; found 323.2.

Step 9. To a solution of 3-(5-bromo-1-ethyl-2-{2-[(1S)-1-methoxyethyl]pyridin-3-yl}indol-3-yl)-2,2-dimethylpropan-1-ol (100 g, 0.22 mol) and 1H-imidazole (30.6 g, 0.45 mol) in DCM (800 mL) was added TBSCl (50.7 g, 0.34 mol) in DCM (200 mL) at 0° C. The reaction was stirred at 25° C. for 2 h. The resulting solution was washed with H₂O (300 mL×3) and brine (200 mL×2), dried over anhydrous sodium sulfate, filtered and concentrated under reduced pressure. The residue was purified with silica gel column chromatography to give (S)-5-bromo-3-(3-((tert-butyldimethylsilyl)oxy)-2,2-dimethylpropyl)-1-ethyl-2-(2-(1-methoxyethyl) pyridin-3-yl)-1H-indole (138 g, 90% yield) as an solid. LCMS (ESI): m/z [M+H] calc'd for C₂₉H₄₃BrN₂O₂Si 558.2; found 559.2.

Step 10. To a stirred solution of Intermediate 1 (50 g, 89.3 mmol) in dioxane (500 mL) was added methyl (2S)-2-{[(benzyloxy) carbonyl]amino}-3-[(2S)-morpholin-2-yl]propanoate from step 1 (31.7 g, 98.2 mmol), RuPhos (16.7 g, 35.7 mmol), Di-mu-chlorobis (2-amino-1,1-biphenyl-2-yl-C,N) dipalladium (II) (2.8 g, 4.4 mmol) and cesium carbonate (96 g, 295 mmol) followed by RuPhos-Pd-G2 (3.5 g, 4.4 mmol) at 105° C. under an N₂ atmosphere. The reaction mixture was stirred for 6 h at 105° C. under an N₂ atmosphere. The resulting mixture was filtered, and the filtrate was concentrated under reduced pressure. The residue was purified by prep-TLC chromatography to afford methyl (2S)-2-{[(benzyloxy) carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl) morpholin-2-yl]propanoate (55 g, 73% yield) as a solid. LCMS (ESI): m/z [M+H] calc'd for C₄₅H₆₄N₄O₇Si 800.5; found 801.5.

Step 11. To a solution of methyl (2S)-2-{[(benzyloxy) carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl) morpholin-2-yl]propanoate (10 g, 12 mmol) in THF (270 mL) was added LiOH (1.3 g, 31 mmol) in H₂O (45 mL) at 20° C. The reaction was stirred at 20° C. for 2 h, then treated with 1N HCl to adjust pH to 4˜5 at 0˜5° C. The resulting mixture was extracted with EtOAc (50 mL×2). The combined organic layers were washed with brine and dried over anhydrous Na₂SO₄. The organic phase was then concentrated under reduced pressure to afford (2S)-2-{[(benzyloxy) carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl) morpholin-2-yl]propanoic acid (9.5 g, 97% yield) as a solid. LCMS (ESI): m/z [M+H] calc'd for C₄₄H₆₂N₄O₇Si 786.4; found 787.4.

Step 12. To a stirred solution of (2S)-2-{[(benzyloxy) carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl) morpholin-2-yl]propanoic acid (10 g, 12.7 mmol) in DMF (150 mL), was added methyl(S)-hexahydropyridazine-3-carboxylate (2 g, 14 mmol), then cooled to 0° C., DIPEA (32.8 g, 254 mmol) was added followed by HATU (9.7 g, 25.4 mmol) at 0˜5° C. The reaction mixture was stirred at 0˜5° C. for 1 h. The resulting mixture was diluted with EtOAc (500 mL) and H₂O (200 mL). The organic layer was separated and washed with H₂O (100 ml×2) and brine (100 mL), dried over anhydrous sodium sulfate. The solution was filtered and concentrated under reduced pressure, and the residue was purified by silica gel column chromatography to afford methyl(S)-1-((S)-2-(((benzyloxy) carbonyl) amino)-3-((S)-4-(3-(3-((tert-butyldimethylsilyl)oxy)-2,2-dimethylpropyl)-1-ethyl-2-(2-((S)-1-methoxyethyl) pyridin-3-yl)-1H-indol-5-yl) morpholin-2-yl) propanoyl) hexahydropyridazine-3-carboxylate (8 g, 70% yield) as a solid. LCMS (ESI): m/z [M+H] calc'd for C₅₀H₇₂N₆O₈Si 912.5; found 913.4.

Step 13. A solution of methyl(S)-1-((S)-2-(((benzyloxy) carbonyl) amino)-3-((S)-4-(3-(3-((tert-butyldimethylsilyl)oxy)-2,2-dimethylpropyl)-1-ethyl-2-(2-((S)-1-methoxyethyl) pyridin-3-yl)-1H-indol-5-yl) morpholin-2-yl) propanoyl) hexahydropyridazine-3-carboxylate (8.5 g, 9 mmol) in THF (8 mL) was added a mixture of tetrabutylammonium fluoride (1M in THF, 180 mL, 180 mmol) and AcOH (11 g, 200 mmol) at 20° C. The reaction mixture was stirred at 75° C. for 3 h. The resulting mixture was diluted with EtOAc (150 mL) and washed with H₂O (20 mL×6). The organic phase was concentrated under reduced pressure to give methyl(S)-1-((S)-2-(((benzyloxy) carbonyl) amino)-3-((S)-4-(1-ethyl-3-(3-hydroxy-2,2-dimethylpropyl)-2-(2-((S)-1-methoxyethyl) pyridin-3-yl)-1H-indol-5-yl) morpholin-2-yl) propanoyl) hexahydropyridazine-3-carboxylate (7.4 g, 100% yield) as solid. LCMS (ESI): m/z [M+H] calc'd for C₄₄H₅₈N₆O₈ 799.4; found 798.4.

Step 14. To a solution of methyl(S)-1-((S)-2-(((benzyloxy) carbonyl) amino)-3-((S)-4-(1-ethyl-3-(3-hydroxy-2,2-dimethylpropyl)-2-(2-((S)-1-methoxyethyl) pyridin-3-yl)-1H-indol-5-yl) morpholin-2-yl) propanoyl) hexahydropyridazine-3-carboxylate (8 g, 10 mmol) in THF (200 mL) was added lithium hydroxide (600 mg, 25 mmol) in H₂O (30 mL). The reaction mixture was stirred at 20° C. for 1 h, then treated with 1N HCl to adjust pH to 4˜5 at 0˜5° C., and extracted with EtOAc (500 mL×2). The organic phase was washed with brine, and concentrated under reduced pressure to afford(S)-1-((S)-2-(((benzyloxy) carbonyl) amino)-3-((S)-4-(1-ethyl-3-(3-hydroxy-2,2-dimethylpropyl)-2-(2-((S)-1-methoxyethyl) pyridin-3-yl)-1H-indol-5-yl) morpholin-2-yl) propanoyl) hexahydropyridazine-3-carboxylic acid (8 g, crude) as a solid. LCMS (ESI): m/z [M+H] calc'd for C₄₃H₅₆N₆O₈ 784.4; found 785.4.

Step 15. To a stirred solution of(S)-1-((S)-2-(((benzyloxy) carbonyl) amino)-3-((S)-4-(1-ethyl-3-(3-hydroxy-2,2-dimethylpropyl)-2-(2-((S)-1-methoxyethyl) pyridin-3-yl)-1H-indol-5-yl) morpholin-2-yl) propanoyl) hexahydropyridazine-3-carboxylic acid (8 g, 10.2 mmol) and DIPEA (59 g, 459 mmol) in DCM (800 mL) was added EDCI (88 g, 458 mmol) and HOBT (27.6 g, 204 mmol) at 25° C. under an argon atmosphere. The reaction mixture was stirred at 25° C. for 16 h. The resulting mixture was concentrated under reduced pressure, and the residue was purified by silica gel column chromatography to afford benzyl ((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) carbamate (5 g, 66% yield) as a solid; LCMS (ESI): m/z [M+H] calc'd for C₄₃H₅₄N₆O₇ 766.4; found 767.4.

Step 16. To a solution of benzyl ((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) carbamate (400 mg, 0.5 mmol) in MeOH (20 mL) was added Pd/C (200 mg) and ammonium acetate (834 mg, 16 mmol) at 20° C. under an H₂ atmosphere and the mixture was stirred for 2 h. Then resulting mixture was filtered and concentrated under reduced pressure. The residue was redissolved in DCM (20 mL) and washed with H₂O (5 ml×2), then concentrated under reduced pressure to afford (22S,6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione (320 mg, 97% yield) as a solid. LCMS (ESI): m/z [M+H] calc'd for C₃₅H₄₈N₆O₅ 632.4; found 633.3.

Step 17. To a solution of the (22S,6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione (50 mg, 0.079 mmol), N-(1-(4-(dimethylamino)-4-methylpent-2-ynoyl)-4-fluoropiperidine-4-carbonyl)-N-methyl-L-valine (47 mg, 0.12 mmol) in DMF (2 mL) stirred at 0° C. was added HATU (36 mg, 0.09 mmol) and DIPEA (153 mg, 1.2 mmol) dropwise. The reaction was stirred at 0° C. for 1 h. The resulting mixture was purified by reverse phase to afford 1-(4-(dimethylamino)-4-methylpent-2-ynoyl)-N-((2S)-1-(((22S,6³S,4S)-11-ethyl-1²-(2-((S)-1-methoxyethyl) pyridin-3-yl)-10, 10-dimethyl-5,7-dioxo-6¹,6²,6³,6⁴,6⁵,6⁶-hexahydro-11H-8-oxa-2 (4,2)-morpholina-1 (5,3)-indola-6 (1,3)-pyridazinacycloundecaphane-4-yl) amino)-3-methyl-1-oxobutan-2-yl)-4-fluoro-N-methylpiperidine-4-carboxamide (11.9 mg, 13.9% yield) as a solid. ¹H NMR (400 MHZ, CD₃OD) δ 8.71 (dd, J=4.8, 1.7 Hz, 1H), 7.86 (d, J=7.8 Hz, 1H), 7.51 (dd, J=7.8, 4.8 Hz, 1H), 7.39 (d, J=8.9 Hz, 1H), 7.15-7.04 (m, 2H), 5.67 (d, J=8.8 Hz, 1H), 4.62 (d, J=11.2 Hz, 1H), 4.46 (d, J=12.4 Hz, 1H), 4.39-4.27 (m, 2H), 4.23 (d, J=6.1 Hz, 1H), 4.17-4.08 (m, 1H), 3.93 (s, 2H), 3.86 (s, 1H), 3.84-3.76 (m, 2H), 3.74-3.65 (m, 2H), 3.63-3.51 (m, 2H), 3.27-3.23 (m, 1H), 3.22-3.11 (m, 6H), 3.0-2.89 (m, 2H), 2.85-2.75 (m, 2H), 2.74-2.55 (m, 2H), 2.36 (d, J=8.2 Hz, 6H), 2.32-2.21 (m, 2H), 2.20-2.02 (m, 5H), 1.92 (d, J=12.5 Hz, 2H), 1.69 (dd, J=43.8, 12.6 Hz, 2H), 1.46 (dt, J=8.0, 4.9 Hz, 9H), 1.03 (d, J=3.5 Hz, 3H), 0.90 (dd, J=48.3, 6.5 Hz, 6H), 0.77 (d, J=3.0 Hz, 3H), 0.69 (s, 3H). LCMS (ESI): m/z [M+H] calc'd for C₅₅H₇₈FN₉O₈ 1011.6; found 1012.5.

### Example A375. Synthesis of (2R)-2-(((1-(4-(dimethylamino)-4-methylpent-2-ynoyl) azetidin-3-yl)oxy) methyl)-N-((22S,6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide

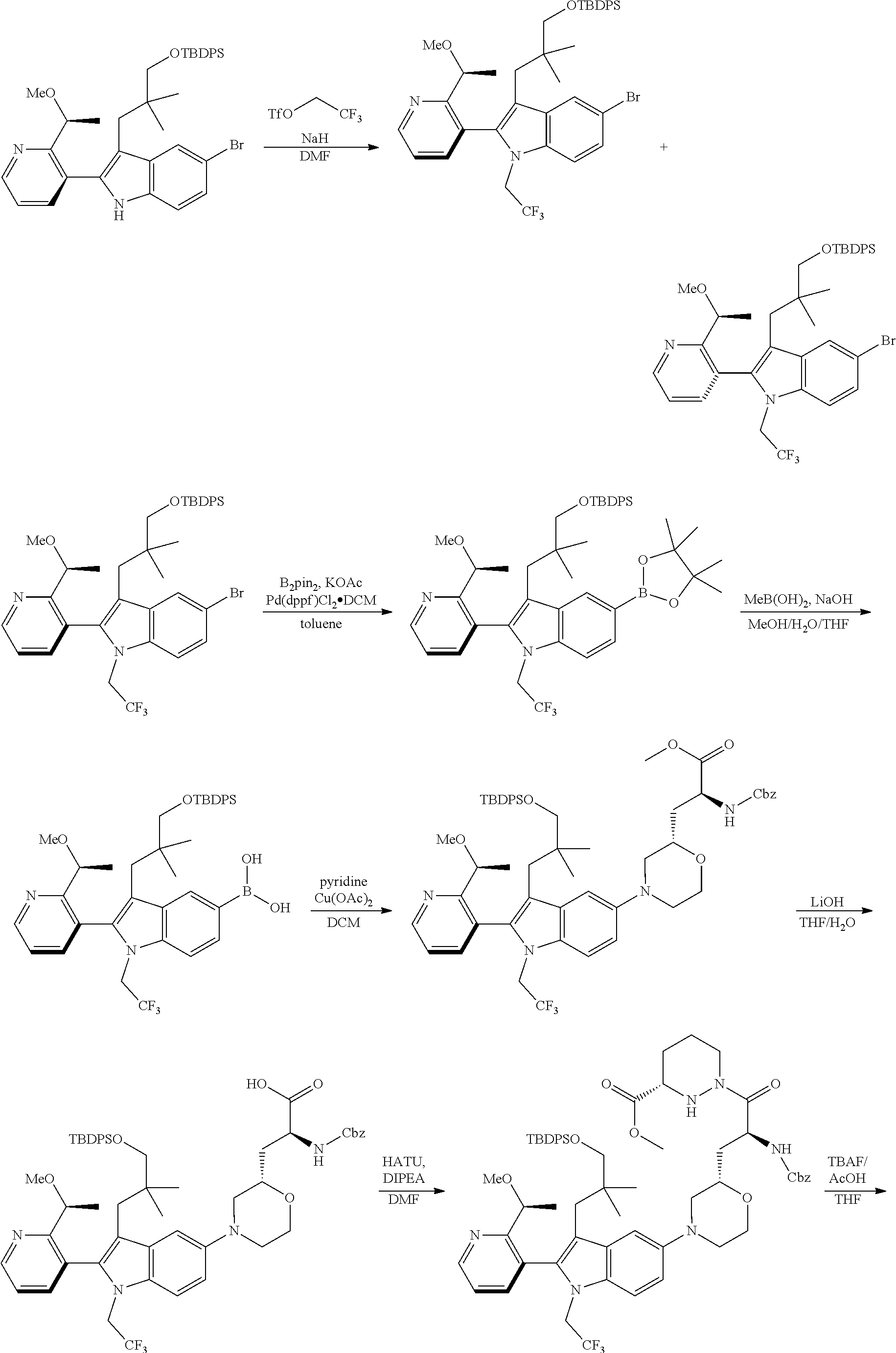

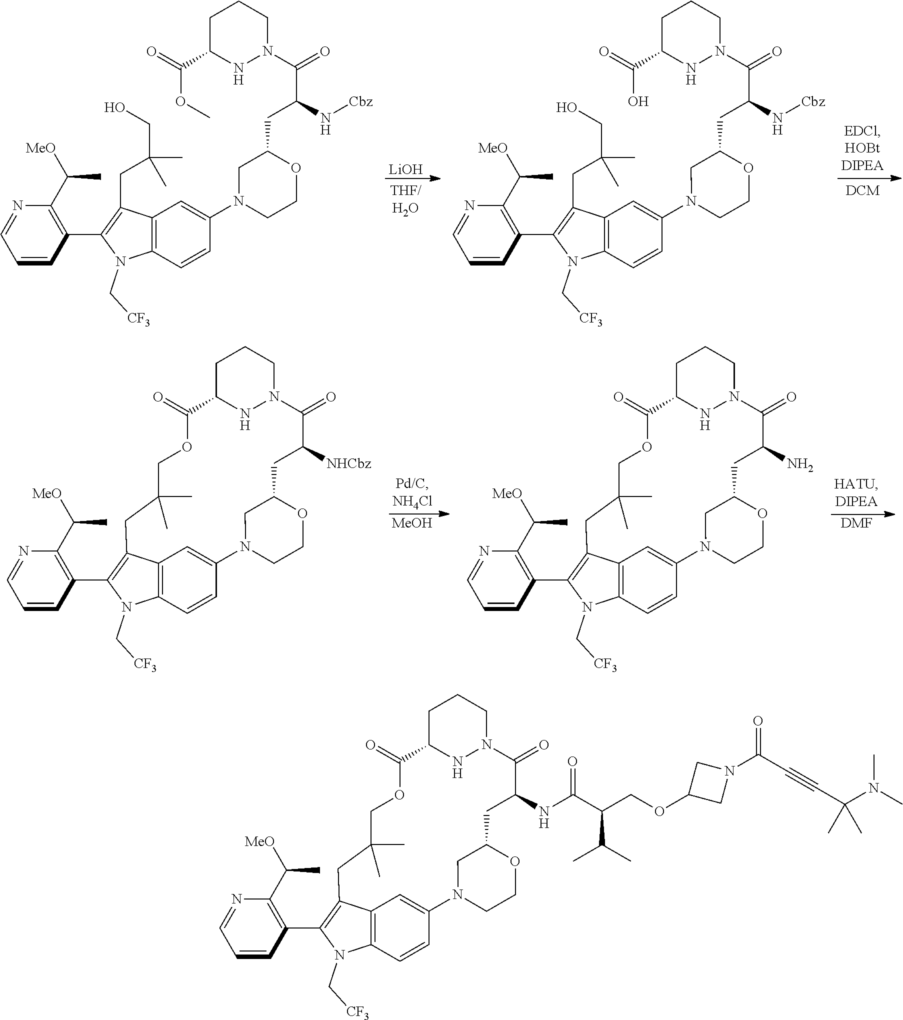

Step 1. To a mixture of 5-bromo-3-{3-[(tert-butyldiphenylsilyl)oxy]-2,2-dimethylpropyl}-2-{2-[(1S)-1-methoxyethyl]pyridin-3-yl}-1H-indole (10.0 g, 15.2 mmol) in anhydrous DMF (120 mL) at 0° C. under an atmosphere of N₂ was added NaH, 60% dispersion in oil (1.2 g, 30.4 mmol) and 2,2,2-trifluoroethyl trifluoromethanesulfonate (35.4 g, 152 mmol). The mixture was stirred at 0° C. for 1 h, then saturated NH₄Cl (30 mL) added and the mixture extracted with EtOAc (100 ml×3). The combined organic layers were washed with brine, dried over anhydrous Na₂SO₄ and filtered. The filtrate was concentrated under reduced pressure and the residue was purified by silica gel column chromatography to give (S)-5-bromo-3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indole (8 g) as an oil and the other atropisomer (6 g, 48% yield) as an oil. LCMS (ESI): m/z [M+H] calc'd for C₃₉H₄₄BrF₃N₂O₂Si 736.2; found 737.1.

Step 2. To a mixture of(S)-5-bromo-3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indole (7.2 g, 9.7 mmol) in toulene (80 mL) under an atmosphere of N₂ was added 4,4,4′, 4′, 5,5,5′, 5′-octamethyl-2,2′-bi (1,3,2-dioxaborolane) (2.7 g, 10.6 mmol), KOAc (1.9 g, 19.4 mmol) and Pd(dppf)Cl₂ DCM (0.8 g, 0.1 mmol). The mixture was heated to 90° C. and stirred for 8 h, then saturated NH₄Cl (30 mL) added and the mixture extracted with EtOAc (40 mL×3). The combined organic layers were dried over anhydrous Na₂SO₄ and filtered. The filtrate was concentrated under reduced pressure and the residue was purified by silica gel column chromatography to give (S)-3-(3-((tert-butyldiphenylsilyl)oxy)-2,2-dimethylpropyl)-2-(2-(1-methoxyethyl) pyridin-3-yl)-5-(4,4,5,5-tetramethyl-1,3,2-dioxaborolan-2-yl)-1-(2,2,2-trifluoroethyl)-1H-indole (6.1 g, 64% yield) as an oil. LCMS (ESI): m/z [M+H] calc'd for C₄₅H₅₆BF₃N₂O₄Si 784.4; found 785.3.

Step 3. To a mixture of(S)-3-(3-((tert-butyldiphenylsilyl)oxy)-2,2-dimethylpropyl)-2-(2-(1-methoxyethyl) pyridin-3-yl)-5-(4,4,5,5-tetramethyl-1,3,2-dioxaborolan-2-yl)-1-(2,2,2-trifluoroethyl)-1H-indole (33 g, 42 mmol) in THF (120 mL) and MeOH (330 mL) at 0° C. under an atmosphere of N₂ was added MeB(OH)₂ (50.4 g, 841 mmol), then a mixture of NaOH (33.6 g, 841 mmol) in H₂O (120 mL). The mixture was warmed to room temperature and stirred for 16 h, then concentrated under reduced pressure. H₂O (500 mL) was added to the residue and the mixture extracted with EtOAc (300 mL×3). The combined organic layers were washed with brine (300 mL), H₂O (300 mL), then concentrated under reduced pressure and the residue was purified by silica gel column chromatography to give (S)-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl) boronic acid (20 g, 68% yield) as a solid. LCMS (ESI): m/z [M+H] calc'd for C₃₉H₄₆BF₃N₂O₄Si 702.3; found 703.3.

Step 4. Note: Three reactions were run in parallel—the yield reflects the sum of the products.

A mixture of(S)-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl) boronic acid (1.85 g, 5.6 mmol) and methyl(S)-2-(((benzyloxy) carbonyl) amino)-3-((S)-morpholin-2-yl) propanoate in DCM (150 mL) under air was added pyridine (1.35 g, 16.9 mmol) and Cu (OAc) 2 (2.0 g, 11.3 mmol). The mixture was stirred for 48 h, then concentrated under reduced pressure. H₂O (300 mL) was added to the residue and the mixture was extracted with EtOAc (300 mL×2). The combined organic layers were washed with brine (100 mL), dried over anhydrous Na₂SO₄ and filtered. The filtrate was concentrated under reduced pressure and the residue was silica gel column chromatography to give methyl(S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoate (9.2 g, 55% yield) as a solid. LCMS (ESI): m/z [M/2+H] calc'd for C₅₅H₆₅F₃N₄O₇Si 490.2; found 490.3.

Step 5. To a mixture of methyl(S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoate (10.8 g, 11.0 mmol) in THF (50 mL) was added LiOH (528 mg, 22 mmol) in H₂O (10 mL). The mixture was stirred for 1 h, then cooled to 0-5° C. and acidified to pH˜7 using 2N HCl (10 mL). The mixture was extracted with DCM (100 mL×2) and the combined organic layers were dried over Na₂SO₄ and filtered. The filtrate was concentrated under reduced pressure to give (S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoic acid (10.6 g, 100% yield) as a solid. LCMS (ESI): m/z [M/2+H] calc'd for C₅₄H₆₃F₃N₄O₇Si 483.2; found 483.3.

Step 6. To a mixture of(S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoic acid (10.6 g, 11.0 mmol) and methyl (3S)-1,2-diazinane-3-carboxylate (15.8 g, 22.0 mmol) in DMF (150 mL) at 0° C. was added DIPEA (28.4 g, 220 mmol) and HATU (8.4 g, 22.0 mmol). The mixture was stirred at 0˜5° C. for 1 h, then EtOAc (500 mL) was added and the mixture was washed with H₂O (200 ml×2), brine (100 mL), dried over anhydrous Na₂SO₄ and filtered. The filtrate was concentrated under reduced pressure and the residue was purified by silica gel column chromatography to give methyl(S)-1-((S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylate (11 g, 90% yield) as a solid. LCMS (ESI): m/z [M/2+H] calc'd for C₆₀H₇₃F₃N₆O₈Si 546.3; found 546.3.

Step 7. To a mixture of methyl(S)-1-((S)-2-(((benzyloxy) carbonyl) amino)-3-((S)-4-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylate (11.0 g, 10.1 mmol) in THF (10 mL) was added a mixture of AcOH (21.2 g, 353 mmol) and 1M TBAF in THF (300 mL, 300 mmol). The mixture was heated to 80° C. and stirred for 16 h, then concentrated under reduced pressure. EtOAc (800 mL) was added to the residue and the mixture was washed with H₂O (80 mL×6), concentrated under reduced pressure and the residue was purified by preparative-HPLC to give methyl(S)-1-((S)-2-(((benzyloxy) carbonyl) amino)-3-((S)-4-(3-(3-hydroxy-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylate (7.9 g, 91% yield) as a solid. LCMS (ESI): m/z [M+H] calc'd for C₄₄H₅₅F₃N₆O₈ 852.4; found 853.3.

Step 8. To a mixture of methyl(S)-1-((S)-2-(((benzyloxy) carbonyl) amino)-3-((S)-4-(3-(3-hydroxy-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylate (7.9 g, 9.3 mmol) in THF (50 mL) was added LiOH (443 mg, 18.5 mmol) in H₂O (10 mL). The mixture was stirred for 1 h, then cooled to 0-5° C. and acidified to ˜pH 7 with 2N HCl (9 mL). The mixture was extracted with DCM (100 mL×2) and the combined organic layers were dried over Na₂SO₄ and filtered. The filtrate was concentrated under reduced pressure to give (S)-1-((S)-2-(((benzyloxy) carbonyl) amino)-3-((S)-4-(3-(3-hydroxy-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylic acid (7.6 g, 98% yield) as a solid. LCMS (ESI): m/z [M+H] calc'd for C₄₃H₅₃F₃N₆O₈ 838.4; found 839.3.

Step 9. To a mixture of(S)-1-((S)-2-(((benzyloxy) carbonyl) amino)-3-((S)-4-(3-(3-hydroxy-2,2-dimethylpropyl)-2-(2-((S)-1-methoxyethyl) pyridin-3-yl)-1-(2,2,2-trifluoroethyl)-1H-indol-5-yl) morpholin-2-yl) propanoyl) hexahydropyridazine-3-carboxylic acid (7.6 g, 9.0 mmol) and DIPEA (52.3 g, 405 mmol) in DCM (800 mL) under an atmosphere of Ar was added EDCI (77.6 g, 405 mmol) and HOBT (12 g, 90 mmol). The mixture was stirred for 16 h, then concentrated under reduced pressure. The residue was diluted with EtOAc (500 mL), washed with H₂O (100 mL×2) and filtered. The organic layer was concentrated under reduced pressure and the residue was purified by silica gel column chromatography to give benzyl ((22S,6³S,4S)-12-(2-((S)-1-methoxyethyl) pyridin-3-yl)-10, 10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) carbamate (6.1 g, 74% yield) as a solid. LCMS (ESI): m/z [M+H] calc'd for C₄₃H₅₁F₃N₆O₇ 820.4; found 821.3.

Step 10. To a mixture of benzyl ((22S,6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) carbamate (700 mg, 0.85 mmol) in MeOH (30 mL) was added 10% Pd on C(317 mg) and NH₄Cl (909 mg). The mixture was stirred under an atmosphere of H₂ (1 atm) for 16 h, then filtered through Celite and the filter cake was washed with MeOH (150 mL). The filtrate was concentrated under reduced pressure, DCM (20 mL) was added to the residue and the mixture was washed with saturated NaHCO₃ (20 mL×3). The organic layer was concentrated under reduced pressure to give (22S,6³S,4S)-4-amino-12-(2-((S)-1-methoxyethyl) pyridin-3-yl)-10, 10-dimethyl-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione (660 mg, 95% yield) as a solid. LCMS (ESI): m/z [M+H] calc'd for C₃₅H₄₅F₃N₆O₅ 686.3; found 687.3; ¹H NMR (400 MHz, CDCl₃) δ 8.80 (dd, J=4.7, 1.7 Hz, 1H), 7.66 (d, J=7.4 Hz, 1H), 7.43-7.30 (m, 2H), 7.12-7.01 (m, 2H), 4.90-4.83 (m, 1H), 4.68 (d, J=12.5 Hz, 1H), 4.57 (dd, J=16.2, 8.1 Hz, 1H), 4.24 (q, J=6.1 Hz, 1H), 4.08 (d, J=10.6 Hz, 1H), 3.97-3.82 (m, 4H), 3.80-3.68 (m, 2H), 3.55 (d, J=11.6 Hz, 1H), 3.21 (d, J=9.4 Hz, 1H), 2.93 (dd, J=19.9, 9.3 Hz, 3H), 2.66 (t, J=11.6 Hz, 1H), 2.47 (d, J=14.5 Hz, 1H), 2.19-2.04 (m, 4H), 1.96 (d, J=13.6 Hz, 2H), 1.80-1.71 (m, 2H), 1.66-1.59 (m, 1H), 1.47 (d, J=6.1 Hz, 3H), 0.88 (s, 3H), 0.42 (s, 3H).

Step 11. To a mixture of (22S,6³S,4S)-4-amino-12-(2-((S)-1-methoxyethyl) pyridin-3-yl)-10,10-dimethyl-1¹-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione (300 mg, 0.4 mmol), (2R)-2-[({1-[4-(dimethylamino)-4-methylpent-2-ynoyl]azetidin-3-yl}oxy) methyl]-3-methylbutanoic acid (157 mg, 0.48 mmol) and DIPEA (569.0 mg, 0.4 mmol) in DMF (5 mL) at 0° C. was added HATU (217 mg, 0.57 mmol). The mixture was stirred at 0° C. for 0.5 h, then diluted with H₂O and extracted with EtOAc (20 mL×3). The combined organic layers were washed with brine (20 mL×3), dried over Na₂SO₄ and filtered. The filtrate was concentrated under reduced pressure and the residue was purified by preparative-TLC to give (2R)-2-(((1-(4-(dimethylamino)-4-methylpent-2-ynoyl) azetidin-3-yl)oxy) methyl)-N-((22S,6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)- pyridazinacycloundecaphane-4-yl)-3-methylbutanamide (200 mg, 46% yield) as a solid. LCMS (ESI): m/z [M+H] calc'd for C₅₂H₇₁F₃N₈O₈ 992.5; found 993.4; ¹H NMR (400 MHZ, CD₃OD) δ 8.74 (m, 1H), 8.00 (m, 1H), 7.85 (d, J=7.7 Hz, 1H), 7.60-7.43 (m, 2H), 7.14 (t, J=9.5 Hz, 2H), 5.63 (s, 1H), 5.06 (m, 1H), 4.64 (s, 1H), 4.52-4.31 (m, 3H), 4.27-4.05 (m, 3H), 3.97 (m, 1H), 3.92-3.66 (m, 6H), 3.59 (m, 2H), 3.46 (m, 2H), 3.25 (d, J=5.3 Hz, 3H), 3.07-2.89 (m, 2H), 2.86-2.59 (m, 3H), 2.38-2.32 (m, 3H), 2.28 (s, 3H), 2.13 (m, 2H), 2.03-1.51 (m, 6H), 1.50-1.41 (m, 6H), 1.38 (d, J=7.5 Hz, 3H), 0.98 (t, J=8.7 Hz, 6H), 0.89 (t, J=6.4 Hz, 3H), 0.54 (d, J=8.4 Hz, 3H).

### Example A722. Synthesis of 1-acryloyl-N-((2S)-1-(((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) amino)-3-methyl-1-oxobutan-2-yl)-4-fluoro-N-methylpiperidine-4-carboxamide

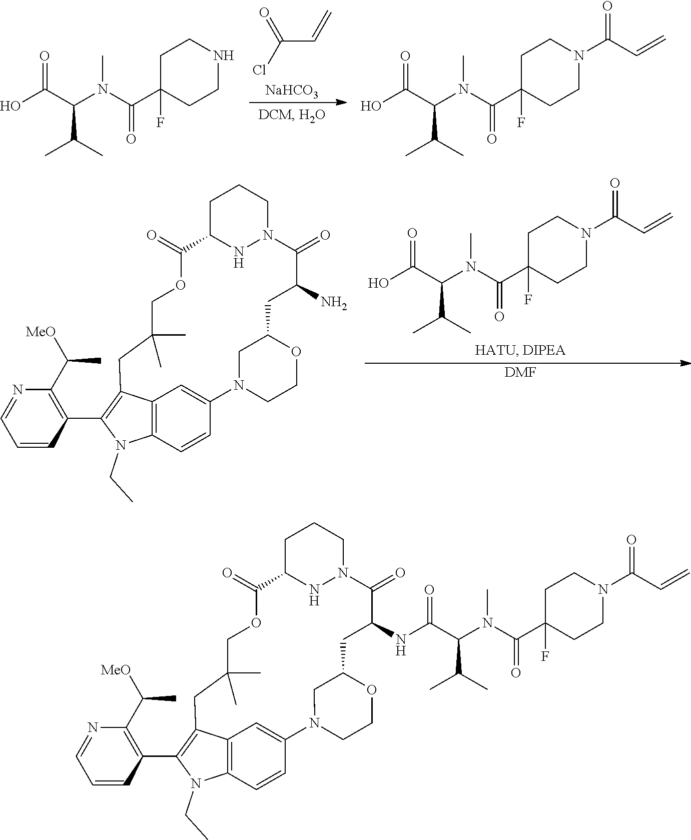

Step 1. To a mixture of N-(4-fluoropiperidine-4-carbonyl)-N-methyl-L-valine (190 mg, 0.73 mmol) and NaHCO₃ (306 mg, 3.6 mmol) in DCM (2 mL) and H₂O (1 mL) at −10° C. was added prop-2-enoyl chloride (132 mg, 1.45 mmol). The mixture was stirred at 0-5° C. for 1 h, then diluted with DCM (20 mL) and washed with H₂O (20 mL×2), brine (20 mL) and the organic layer dried over Na₂SO₄ and filtered. The filtrate was concentrated under reduced pressure and the residue was purified by silica gel column chromatography to give N-(1-acryloyl-4-fluoropiperidine-4-carbonyl)-N-methyl-L-valine (120 mg, 52% yield) as a solid. LCMS (ESI): m/z [M+H] calc'd for C₁₅H₂₃FN₂O₄ 314.2; found 315.2.

Step 2. To a mixture of (6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione (153 mg, 0.24 mmol), N-(1-acryloyl-4-fluoropiperidine-4-carbonyl)-N-methyl-L-valine (106 mg, 0.34 mmol) in DMF (2 mL) at 5° C. was added HATU (110 mg, 0.29 mmol) and DIPEA (468 mg, 3.6 mmol) dropwise. The mixture was stirred at 5° C. for 1 h, then purified by preparative-HPLC to give 1-acryloyl-N-((2S)-1-(((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl) amino)-3-methyl-1-oxobutan-2-yl)-4-fluoro-N-methylpiperidine-4-carboxamide (69.5 mg, 29% yield) as a solid. LCMS (ESI): m/z [M+H] calc'd for C₅₀H₆₉FN₈O₈ 928.5; found 929.4; ¹H NMR (400 MHZ, CD₃OD) δ 8.71 (d, J=3.2 Hz, 1H), 7.86 (d, J=7.7 Hz, 1H), 7.51 (dd, J=7.7, 4.8 Hz, 1H), 7.39 (d, J=8.9 Hz, 1H), 7.18-7.02 (m, 2H), 6.80 (dd, J=16.8, 10.7 Hz, 1H), 6.23 (d, J=16.8 Hz, 1H), 5.77 (d, J=10.6 Hz, 1H), 5.67 (d, J=6.5 Hz, 1H), 4.61 (d, J=11.1 Hz, 1H), 4.44 (t, J=15.1 Hz, 2H), 4.23 (q, J=6.1 Hz, 1H), 4.18-4.10 (m, 1H), 4.09-4.01 (m, 1H), 3.99-3.83 (m, 3H), 3.83-3.65 (m, 4H), 3.58-3.46 (m, 2H), 3.27 (s, 1H), 3.21-3.11 (m, 6H), 3.00-2.91 (m, 2H), 2.85-2.75 (m, 2H), 2.73-2.64 (m, 1H), 2.62-2.54 (m, 1H), 2.36-2.21 (m, 2H), 2.19-2.01 (m, 5H), 1.92 (d, J=12.7 Hz, 2H), 1.79-1.57 (m, 2H), 1.44 (d, J=6.2 Hz, 3H), 1.04 (t, J=6.3 Hz, 3H), 0.98-0.81 (m, 6H), 0.76 (s, 3H), 0.68 (s, 3H).

### Example A377. Synthesis of (2R)-2-(((1-(4-(dimethylamino)-4-methylpent-2-ynoyl) azetidin-3-yl)oxy) methyl)-N-((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide

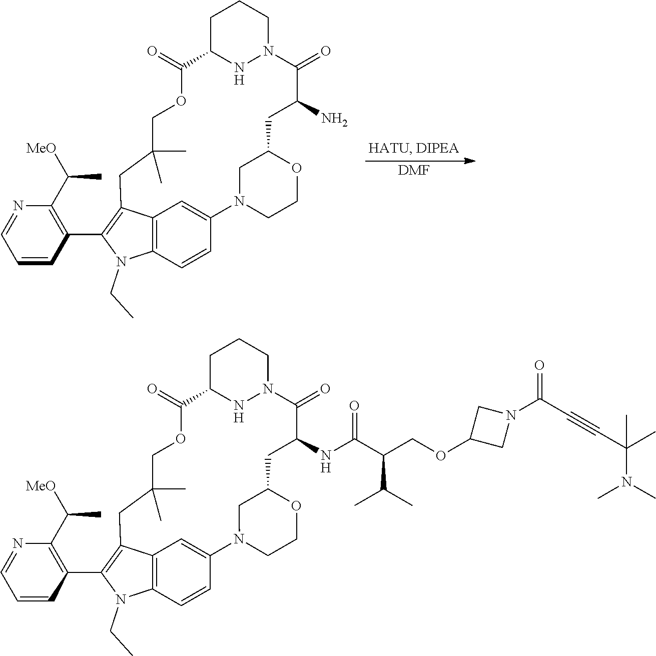

Step 1. (2R)-2-(((1-(4-(dimethylamino)-4-methylpent-2-ynoyl) azetidin-3-yl)oxy) methyl)-N-((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide was synthesized in a manner similar to (2R)-2-(((1-(4-(dimethylamino)-4-methylpent-2-ynoyl) azetidin-3-yl)oxy) methyl)-N-((22S,6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide except (22S,6³S,4S)-4-amino-12-(2-((S)-1-methoxyethyl) pyridin-3-yl)-10, 10-dimethyl-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione was substituted with (22S,6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione and 3- ({1-[4-(dimethylamino)-4-methylpent-2-ynoyl]azetidin-3-yl}oxy) propanoic acid was substituted with (2R)-2-[({1-[4-(dimethylamino)-4-methylpent-2-ynoyl]azetidin-3-yl}oxy) methyl]-3-methylbutanoic acid to give the desired product (25.6 mg, 26% yield) as a solid. LCMS (ESI): m/z [M+H] calc'd for C₅₂H₇₄N₈O₈ 938.6; found 939.5; 1H NMR (400 MHZ, CD₃OD) δ 8.72 (dd, J=4.8, 1.5 Hz, 1H), 7.97 (dd, J=20.0, 6.8 Hz, 1H), 7.87 (dd, J=5.8, 2.6 Hz, 1H), 7.54-7.51 (m, 1H), 7.41 (d, J=8.9 Hz, 1H), 7.14 (dd, J=36.0, 10.4 Hz, 2H), 5.65 (s, 1H), 4.49-4.33 (m, 3H), 4.27-4.08 (m, 4H), 3.96 (d, J=8.6 Hz, 2H), 3.87 (dd, J=10.8, 3.6 Hz, 2H), 3.79 (dd, J=10.8, 7.7 Hz, 3H), 3.69-3.58 (m, 3H), 3.43 (dd, J=23.9, 11.7 Hz, 2H), 3.17 (d, J=21.9 Hz, 3H), 3.00-2.95 (m, 1H), 2.70 (t, J=14.0 Hz, 8H), 2.34-2.24 (m, 1H), 2.05 (d, J=34.4 Hz, 3H), 1.92-1.82 (m, 2H), 1.69-1.62 (m, 5H), 1.57 (d, J=11.5 Hz, 3H), 1.45 (d, J=6.2 Hz, 3H), 1.33 (d, J=12.6 Hz, 1H), 1.05-0.93 (m, 10H), 0.80 (d, J=9.8 Hz, 3H), 0.64 (d, J=12.2 Hz, 2H).

### Example A643. Synthesis of (2R)-2-(((1-(4-(dimethylamino)-4-methylpent-2-ynoyl) piperidin-4-yl)oxy) methyl)-N-((22S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide

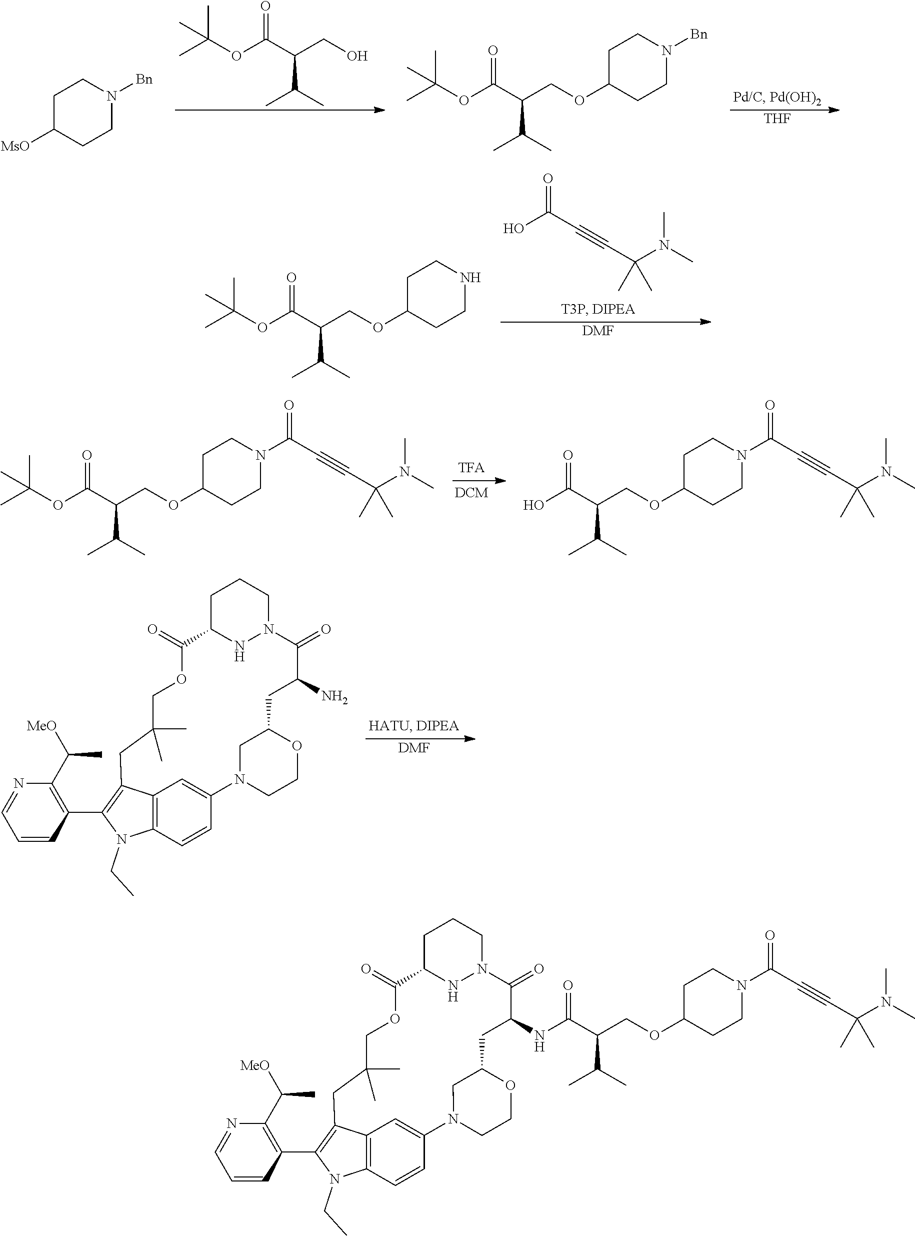

Step 1. A mixture of 1-(1-methylphenyl) piperidin-4-yl methanesulfonate (2 g, 7.4 mmol) and tert-butyl (2R)-2-(hydroxymethyl)-3-methylbutanoate (1.39 g, 7.4 mmol) was stirred at 120° C. for 1 h, then purified by silica gel column chromatography to give tert-butyl (R)-2-(((1-benzylpiperidin-4-yl)oxy) methyl)-3-methylbutanoate (800 mg, 28% yield) as an oil. LCMS (ESI): m/z [M+H] calc'd for C₂₂H₃₅NO₃ 361.3; found 362.3.10 Step 2. A mixture of tert-butyl (R)-2-(((1-benzylpiperidin-4-yl)oxy) methyl)-3-methylbutanoate (700 mg, 1.9 mmol), 10% wet Pd/C (411 mg, 3.9 mmol) and 20% wet Pd (OH) 2/C (542 mg, 3.9 mmol) in THF (30 mL) was stirred under an atmosphere of H₂ (15 psi) for 16 h. The mixture was filtered and the filtrate was concentrated under reduced pressure to give tert-butyl (R)-3-methyl-2-((piperidin-4-yloxy) methyl) butanoate (440 mg, 80% yield) as a an oil. LCMS (ESI): m/z [M+H] calc'd for C₁₅H₂₉NO₃ 271.2; found 272.2.

Step 3. To a mixture of tert-butyl (R)-3-methyl-2-((piperidin-4-yloxy) methyl) butanoate (440 mg, 1.6 mmol) 4-(dimethylamino)-4-methylpent-2-ynoic acid (3.77 g, 24.3 mmol) and DIPEA (2.09 g, 16.2 mmol) in DMF (50 mL) at 0° C. was added T3P (2.57 g, 8.1 mmol). The mixture was stirred at 0° C. for 1 h, then poured into H₂O (50 mL) and extracted with EtOAc (50 mL×3). The combined organic layers were washed with brine, concentrated under reduced pressure and the residue purified by silica gel column chromatography to give tert-butyl (R)-2-(((1-(4-(dimethylamino)-4-methylpent-2-ynoyl) piperidin-4-yl)oxy) methyl)-3-methylbutanoate (190 mg, 27% yield) as an oil. LCMS (ESI): m/z [M+H] calc'd for C₂₃H₄₀N₂O₄ 408.3; found 409.4.

Step 4. To a mixture of tert-butyl (R)-2-(((1-(4-(dimethylamino)-4-methylpent-2-ynoyl) piperidin-4-yl)oxy) methyl)-3-methylbutanoate (180 mg, 0.47 mmol) in DCM (2 mL) was added TFA (1 mL). The mixture was stirred at room temperature for 1 h, then concentrated under reduced pressure to give (R)-2-(((1-(4-(dimethylamino)-4-methylpent-2-ynoyl) piperidin-4-yl)oxy) methyl)-3-methylbutanoic acid (170 mg, 98% yield) as an oil. LCMS (ESI): m/z [M+H] calc'd for C₁₉H₃₂N₂O₄ 352.2; found 353.2.

Step 5. (2R)-2-(((1-(4-(dimethylamino)-4-methylpent-2-ynoyl) piperidin-4-yl)oxy) methyl)-N-((22S,6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide was synthesized in a manner similar to (2R)-2-(((1-(4-(dimethylamino)-4-methylpent-2-ynoyl) azetidin-3-yl)oxy) methyl)-N-((22S,6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-4-yl)-3-methylbutanamide except (22S,6³S,4S)-4-amino-12-(2-((S)-1-methoxyethyl) pyridin-3-yl)-10, 10-dimethyl-11-(2,2,2-trifluoroethyl)-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione was substituted with (22S,6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-morpholina-1 (5,3)-indola-6 (1,3)-pyridazinacycloundecaphane-5,7-dione and 3- ({1-[4-(dimethylamino)-4-methylpent-2-ynoyl]azetidin-3-yl}oxy) propanoic acid was substituted with (2R)-2-[({1-[4-(dimethylamino)-4-methylpent-2-ynoyl]piperidin-4-yl}oxy) methyl]-3-methylbutanoic acid. (101 mg, 42% yield) as a solid. LCMS (ESI): m/z [M+H] calc'd for C₅₄H₇₈N₈O₈ 966.6; found 969.5; ¹H NMR (400 MHZ, CD₃OD) δ 8.70 (d, J=4.8 Hz, 1H), 7.81-7.76 (m, 1H), 7.56-7.46 (m, 1H), 7.43-7.34 (m, 1H), 7.24-7.01 (m, 2H), 5.66-5.54 (m, 1H), 4.50-4.40 (m, 1H), 4.31-4.22 (m, 1H), 4.19-4.08 (m, 1H), 4.02-3.82 (m, 4H), 3.80-3.53 (m, 10H), 3.47-3.34 (m, 2H), 3.26-3.15 (m, 3H), 2.98-2.57 (m, 5H), 2.37-2.30 (m, 3H), 2.27-2.18 (m, 4H), 2.15-2.02 (m, 2H), 2.00-1.80 (m, 4H), 1.78-1.71 (m, 2H), 1.68-1.55 (m, 3H), 1.49-1.37 (m, 6H), 1.35-1.28 (m, 3H), 1.05-0.92 (m, 9H), 0.85-0.72 (m, 3H), 0.68-0.51 (m, 3H).

### Example A328. Synthesis of two atropisomers of (3S)-1-acryloyl-N-((2S)-1-(((6³S,4S)-11-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

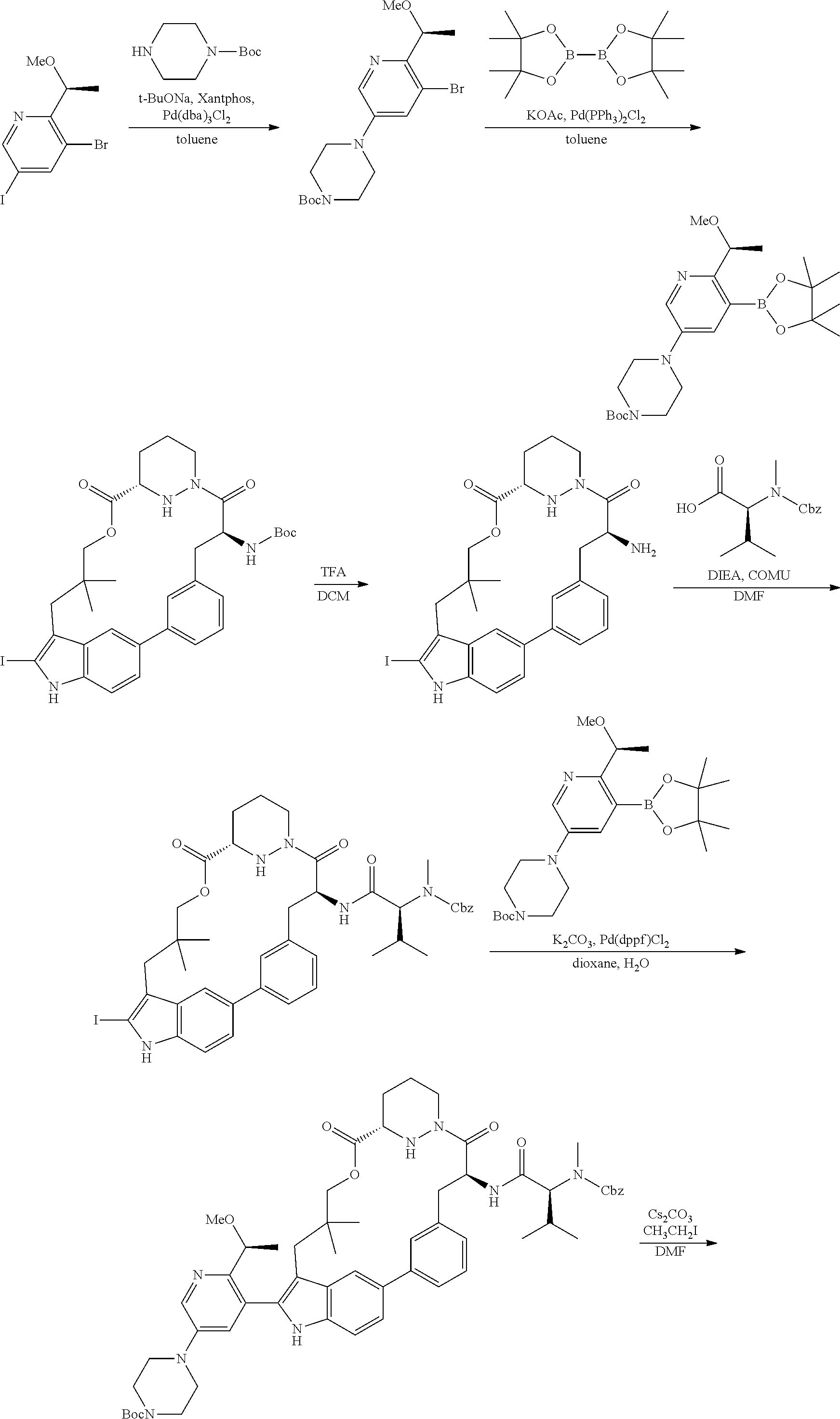

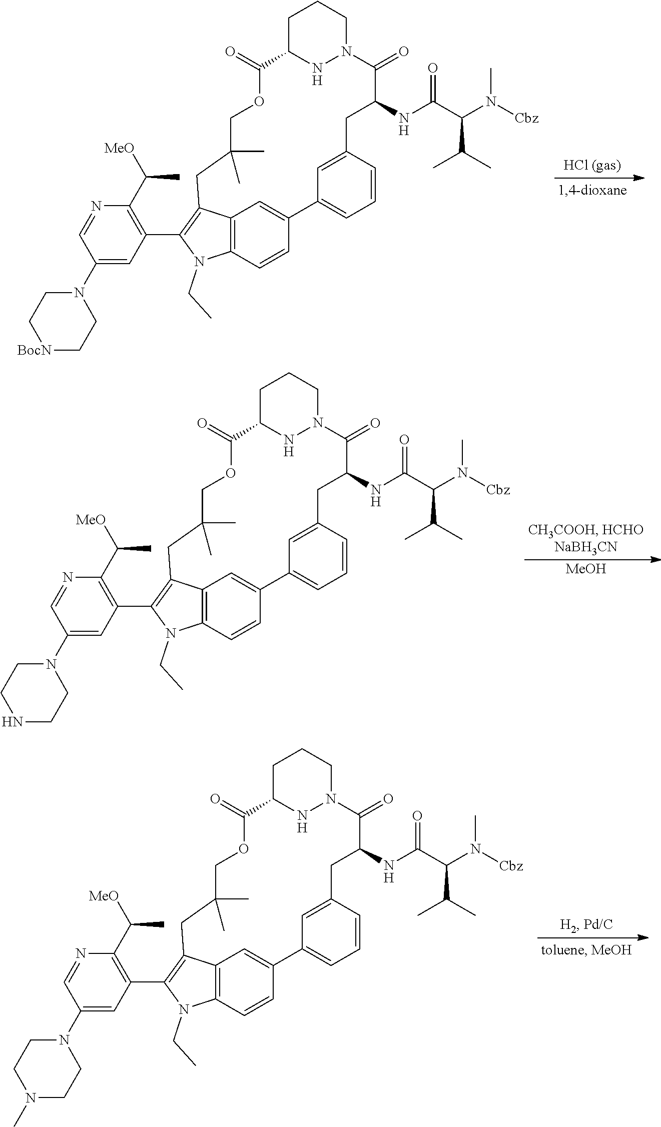

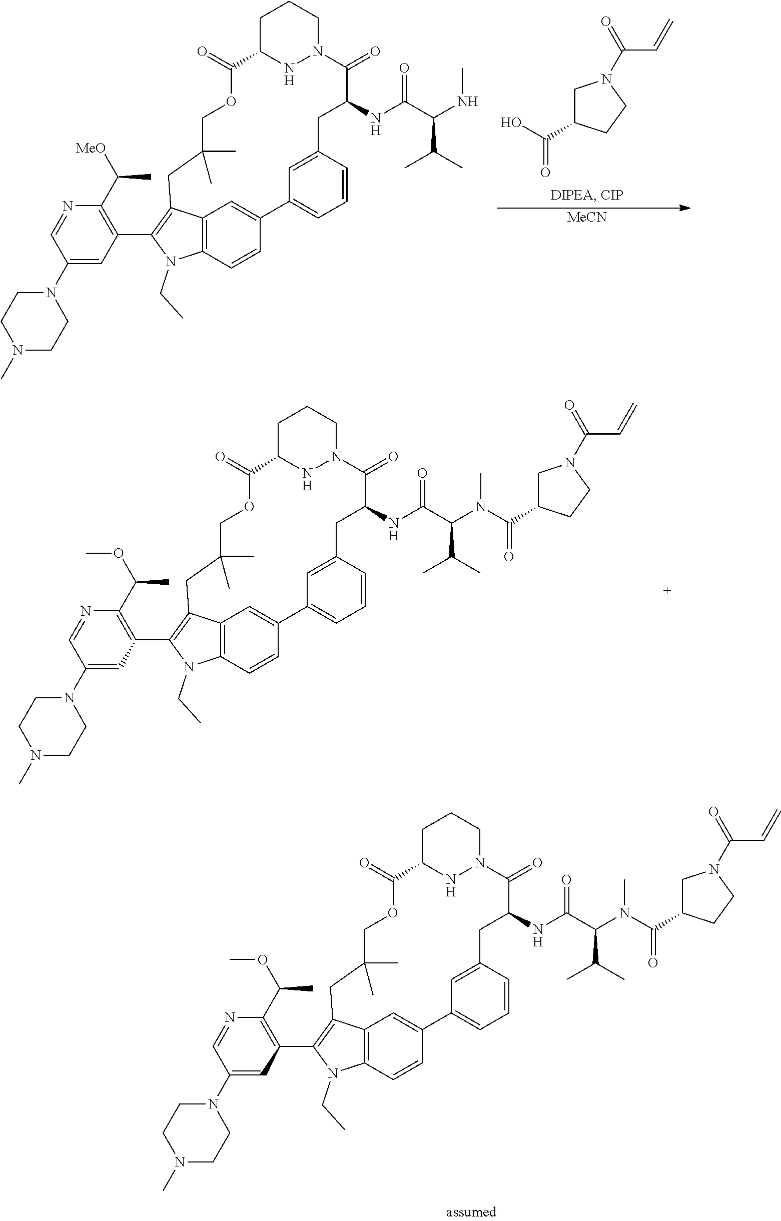

Step 1. To a mixture of 3-bromo-5-iodo-2-[(1S)-1-methoxyethyl]pyridine (2.20 g, 6.4 mmol) and tert-butyl piperazine-1-carboxylate (1.20 g, 6.4 mmol) in toluene (50 mL) under an atmosphere of Ar were added (BuONa (0.74 g, 7.7 mmol) and portion-wise addition of Pd₂(dba)₃ (0.59 g, 0.64 mmol), followed by portion-wise addition of Xantphos (0.74 g, 1.3 mmol). The mixture was heated to 100° C. and stirred for 16 h then H₂O added and the mixture extracted with EtOAc (400 mL×3). The combined organic layers were washed with brine (150 mL×3), dried over anhydrous Na₂SO₄ and filtered. The filtrate was concentrated under reduced pressure and the residue was purified by preparative-HPLC to give tert-butyl 4-[5-bromo-6-[(1 S)-1-methoxyethyl]pyridin-3-yl]piperazine-1-carboxylate (1.7 g, 61% yield) as a solid. LCMS (ESI): m/z [M+H] calc'd for C₁₇H₂₆BrN₃O₃ 399.1; found 400.1.

Step 2. A mixture of tert-butyl 4-[5-bromo-6-[(1S)-1-methoxyethyl]pyridin-3-yl]piperazine-1-carboxylate (1.76 g, 4.4 mmol) and 4,4,4′, 4′, 5,5,5′, 5′-octamethyl-2,2′-bi (1,3,2-dioxaborolane) (1.67 g, 6.6 mmol) in toluene (18 mL) under an atmosphere of Ar were added KOAc (0.95 g, 9.7 mmol) and Pd(PPh₃)₂Cl₂ (0.31 g, 0.44 mmol) in portions. The mixture was heated to 80° C. and stirred for 16 h, then diluted with H₂O and the mixture extracted with EtOAc (500 mL×3). The combined organic layers were washed with brine (100 mL×3), dried over anhydrous Na₂SO₄ and filtered. The filtrate was concentrated under reduced pressure and the residue was purified by silica gel column chromatography to give tert-butyl 4-[6-[(1S)-1-methoxyethyl]-5-(4,4,5,5-tetramethyl-1,3,2-dioxaborolan-2-yl) pyridin-3-yl]piperazine-1-carboxylate (1.4 g, 68% yield) as a solid. LCMS (ESI): m/z [M+H] calc'd for C₂₃H₃₈BN₃O₅ 447.4; found 448.2.

Step 3. To a mixture of tert-butyl ((6³S,4S)-1²-iodo-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.0 g, 1.5 mmol) in DCM (10 mL) at 0° C. under an atmosphere of N₂ was added TFA (5.0 mL, 67.3 mmol) in portions. The mixture was stirred at 0° C. for 1 h then concentrated under reduced pressure and dried azeotropically with toluene (3 mL×3) to give (6³S,4S)-4-amino-12-iodo-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (1.0 g), which was used directly in the next step without further purification. LCMS (ESI): m/z [M+H] calc'd for C₂₇H₃₁IN₄O₃ 586.1; found 587.3.

Step 4. To a mixture of (6³S,4S)-4-amino-12-iodo-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (1.0 g, 1.7 mmol) in DMF (15 mL) at 0° C. under an atmosphere of N₂ were added DIPEA (2.20 g, 17.0 mmol) and (2S)-2-[(benzyloxy) carbonyl](methyl) amino]-3-methylbutanoic acid (0.90 g, 3.4 mmol) in portions, followed by COMU (1.10 g, 2.6 mmol) in portions over 10 min. The mixture was stirred at 0° C. for 1.5 h, then diluted with H₂O and the mixture was extracted with EtOAc (300 mL×3). The combined organic layers were washed with brine (100 mL×3), dried over anhydrous Na₂SO₄ and filtered. The filtrate was concentrated under reduced pressure and the residue was purified by preparative-HPLC to give benzyl ((2S)-1-(((6³S,4S)-1²-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (790 mg, 53% yield) as a solid.

Step 5. To a mixture of benzyl ((2S)-1-(((6³S,4S)-1²-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (480 mg, 0.58 mmol) and tert-butyl 4-[6-[(1S)-1-methoxyethyl]-5-(4,4,5,5-tetramethyl-1,3,2-dioxaborolan-2-yl) pyridin-3-yl]piperazine-1-carboxylate (309 mg, 0.69 mmol) in 1,4-dioxane (8.0 mL) and H₂O (1.6 mL) under an atmosphere of Ar were added K₂CO₃ (199 mg, 1.4 mmol) and Pd(dppf)Cl₂ (42 mg, 0.06 mmol) in portions. The mixture was heated to 70° C. and stirred for 16 h, then diluted with H₂O and extracted with EtOAc (200 mL×3). The combined organic layers were washed with brine (150 mL×3), dried over anhydrous Na₂SO₄ and filtered. The filtrate was concentrated under reduced pressure and the residue was purified by silica gel column chromatography to give tert-butyl 4-(5-((6³S,4S)-4-((S)-2-(((benzyloxy) carbonyl) (methyl) amino)-3-methylbutanamido)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (335 mg, 51% yield) as a solid. LCMS (ESI): m/z [M+H] calc'd for C₅₈H₇₄N₈O₉ 1026.6; found 1027.4.

Step 6. To a mixture of tert-butyl 4-(5-((6³S,4S)-4-((S)-2-(((benzyloxy) carbonyl) (methyl) amino)-3-methylbutanamido)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (335 mg, 0.33 mmol) in DMF (5 mL) at 0° C. under an atmosphere of N₂ were added Cs₂CO₃ (234 mg, 0.72 mmol) and iodoethane (102 mg, 0.65 mmol) in portions. The mixture was warmed to room temperature and stirred for 16 h, then diluted with H₂O and the mixture extracted with EtOAc (100 mL×3). The combined organic layers were washed with brine (50 mL×3), dried over anhydrous Na₂SO₄ and filtered. The filtrate was concentrated under reduced pressure and the residue was purified by preparative-TLC to give tert-butyl 4-(5-((6³S,4S)-4-((S)-2-(((benzyloxy) carbonyl) (methyl) amino)-3-methylbutanamido)-1¹-ethyl-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (320 mg, 84% yield) as a light yellow solid. LCMS (ESI): m/z [M+H] calc'd for C₆₀H₇₈N₈O₉ 1054.6; found 1055.8.

Step 7. A mixture of tert-butyl 4-(5-((6³S,4S)-4-((S)-2-(((benzyloxy) carbonyl) (methyl) amino)-3-methylbutanamido)-1¹-ethyl-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (320 mg) in xx M HCl in 1,4-dioxane (3.0 mL) at 0° C. under an atmosphere of N₂ was stirred at room temperature for 2 h, then concentrated under reduced pressure to give benzyl ((2S)-1-(((6³S,4S)-1¹-ethyl-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate, which was used directly in the next step further purification. LCMS (ESI): m/z [M+H] calc'd for C₅₅H₇₀N₈O₇ 954.5; found 955.3.

Step 8. To a mixture of benzyl ((2S)-1-(((6³S,4S)-1¹-ethyl-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (320 mg, 0.34 mmol) and HCHO (60 mg, 2.0 mmol) in MeOH (3.0 mL) at 0° C. under an atmosphere of N₂ were added NaCNBH₃ (42 mg, 0.67 mmol) and AcOH (60 mg, 1.0 mmol) in portions. The mixture was warmed to room temperature and stirred for 2 h, then diluted with H₂O and the mixture extracted with DCM/MeOH (5:1) (200 mL×3). The combined organic layers were washed with brine (100 mL×3), dried over anhydrous Na₂SO₄ and filtered. The filtrate was concentrated under reduced pressure and the residue was purified by preparative-HPLC to give benzyl ((2S)-1-(((6³S,4S)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (160 mg, 59% yield) as a solid. LCMS (ESI): m/z [M+H] calc'd for C₅₆H₇₂N₈O₇ 968.6; found 969.6.

Step 9. To a mixture of benzyl ((2S)-1-(((6³S,4S)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (160 mg, 0.17 mmol) in toluene (10 mL) and MeOH (1.0 mL) was added Pd/C (130 mg, 1.2 mmol) in portions. The mixture was evacuated and re-filled with H₂ (×3), then stirred under an atmosphere of H₂ for 16 h. The mixture was filtered and the filtrate was concentrated under reduced pressure to give (2S)—N-((6³S,4S)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (140 mg), which was used directly in the next step without further purification. LCMS (ESI): m/z [M+H] calc'd for C₄₈H₆₆N₈O₅ 834.5; found 835.5.

Step 10. To a mixture of (2S)—N-((6³S,4S)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (140 mg, 0.17 mmol) in ACN (2.0 mL) at 0° C. under an atmosphere of N₂ were added DIPEA (433 mg, 3.35 mmol), (3S)-1-(prop-2-enoyl) pyrrolidine-3-carboxylic acid (57 mg, 0.34 mmol) in portions and CIP (70 mg, 0.25 mmol) in portions over 10 min. The mixture was stirred at 0° C. for 1.5 h, then H₂O added and the mixture extracted with EtOAc (150 mL×3). The combined organic layers were washed with brine (100 mL×3), dried over anhydrous Na₂SO₄ and filtered. The filtrate was concentrated under reduced pressure and the residue was purified by preparative-HPLC to give two atropisomers of (3S)-1-acryloyl-N-((2S)-1-(((6³S,4S)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide (40 mg, 24% yield) as a solid and (20 mg, 12% yield) as a solid. LCMS (ESI): m/z [M+H] calc'd for C₅₆H₇₅N₉O₇ 985.6; found 986.7; ¹H NMR (400 MHZ, DMSO-d₆) 8.47 (t, J=2.1 Hz, 1H), 8.00 (d, J=4.7 Hz, 1H), 7.78-7.59 (m, 3H), 7.58-7.48 (m, 1H), 7.42-7.30 (m, 1H), 7.23 (dq, J=8.0, 4.0, 3.5 Hz, 1H), 7.15-7.03 (m, 1H), 6.75-6.50 (m, 1H), 6.18 (dt, J=16.8, 2.7 Hz, 1H), 5.70 (tt, J=9.3, 2.7 Hz, 1H), 5.48-5.23 (m, 1H), 5.06 (dd, J=31.1, 12.3 Hz, 1H), 4.74 (dd, J=11.0, 4.3 Hz, 1H), 4.33-4.15 (m, 2H), 4.01 (ddd, J=36.1, 12.6, 7.6 Hz, 2H), 3.91-3.56 (m, 6H), 3.52-3.39 (m, 2H), 3.31-3.28 (m, 2H), 3.24 (d, J=5.7 Hz, 4H), 3.06 (s, 4H), 2.93 (d, J=9.8 Hz, 2H), 2.81 (d, J=5.4 Hz, 3H), 2.47-2.43 (m, 4H), 2.22 (s, 4H), 2.09 (tq, J=12.0, 7.4, 6.6 Hz, 3H), 1.81 (s, 1H), 1.74 (d, J=11.7 Hz, 1H), 1.56 (d, J=11.7 Hz, 1H), 1.20 (dd, J=6.3, 1.5 Hz, 3H), 1.10 (td, J=7.2, 2.4 Hz, 3H), 1.00-0.86 (m, 6H), 0.86-0.72 (m, 3H), 0.54 (d, J=3.5 Hz, 3H) and LCMS (ESI): m/z [M−H] calc'd for C₅₆H₇₅N₉O₇ 985.6; found 984.4; ¹H NMR (400 MHZ, DMSO-d₆) δ 8.46 (d, J=3.0 Hz, 2H), 7.98 (s, 1H), 7.89-7.83 (m, 1H), 7.76-7.57 (m, 3H), 7.24 (s, 2H), 7.07 (s, 1H), 6.70-6.58 (m, 1H), 6.17 (d, J=16.5 Hz, 1H), 5.73-5.67 (m, 1H), 5.36-5.30 (m, 1H), 4.31-3.97 (m, 6H), 3.83-3.77 (m, 2H), 3.74-3.49 (m, 6H), 3.48-3.41 (m, 1H), 3.40-3.37 (m, 2H) 3.28-3.24 (m, 4H), 3.07 (s, 3H), 2.88-2.82 (m, 1H), 2.80-2.64 (m, 7H), 2.49-2.44 (m, 4H), 2.22 (s, 3H), 2.04 (d, J=26.1 Hz, 3H), 1.85-1.79 (m, 1H), 1.67-1.55 (m, 2H), 1.35 (d, J=6.1 Hz, 3H), 1.27-1.22 (m, 1H), 1.05-0.93 (m, 4H), 0.89 (d, J=6.9 Hz, 2H), 0.79 (d, J=12.4 Hz, 5H), 0.73 (d, J=6.5 Hz, 1H), 0.56 (s, 3H).

### Example A542. The synthesis of 1-(4-(dimethylamino)-4-methylpent-2-ynoyl)-4-fluoro-N-((2S)-1-(((6³S,4S)-1²-(2-((S)-1-methoxyethyl)-5-(4-methylpiperazin-1-yl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperidine-4-carboxamide

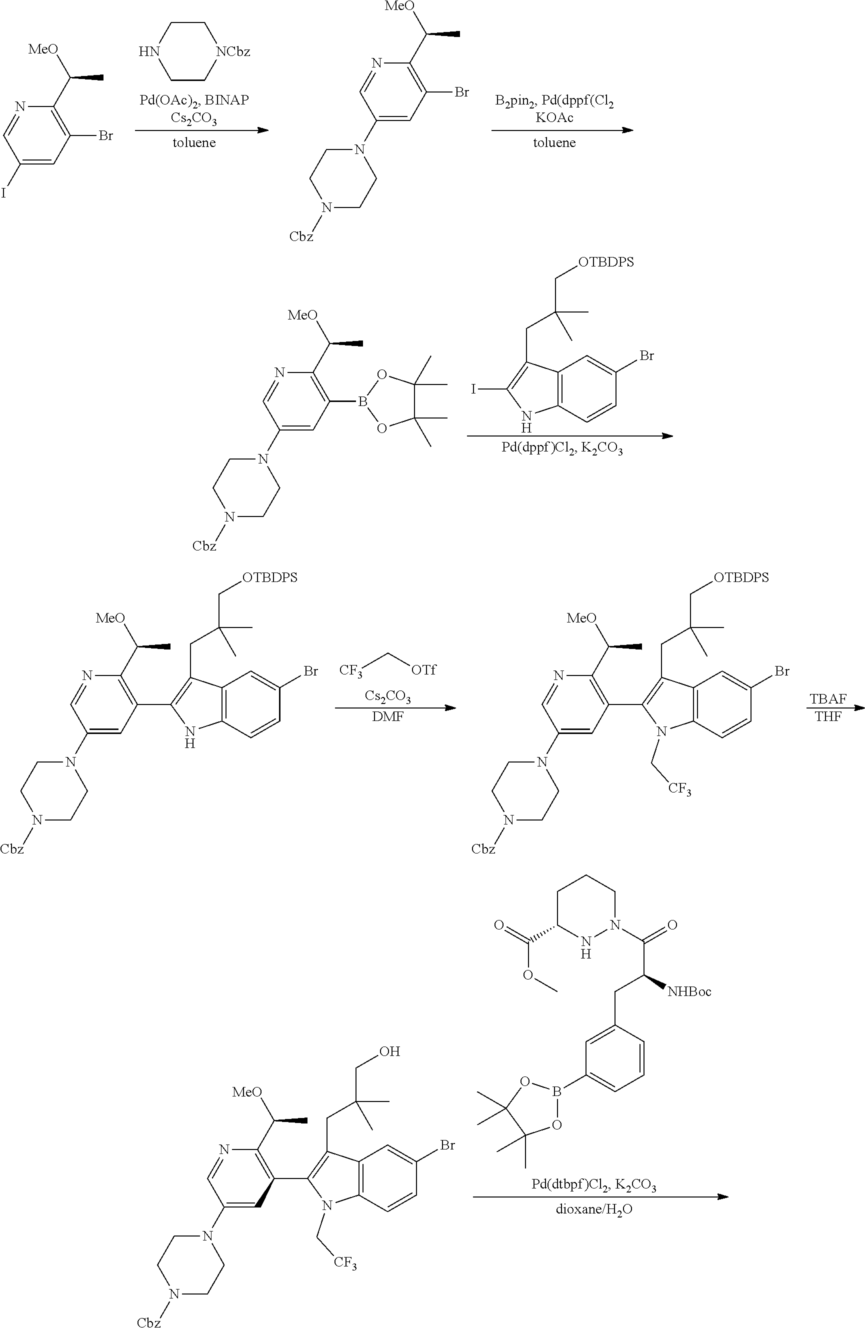

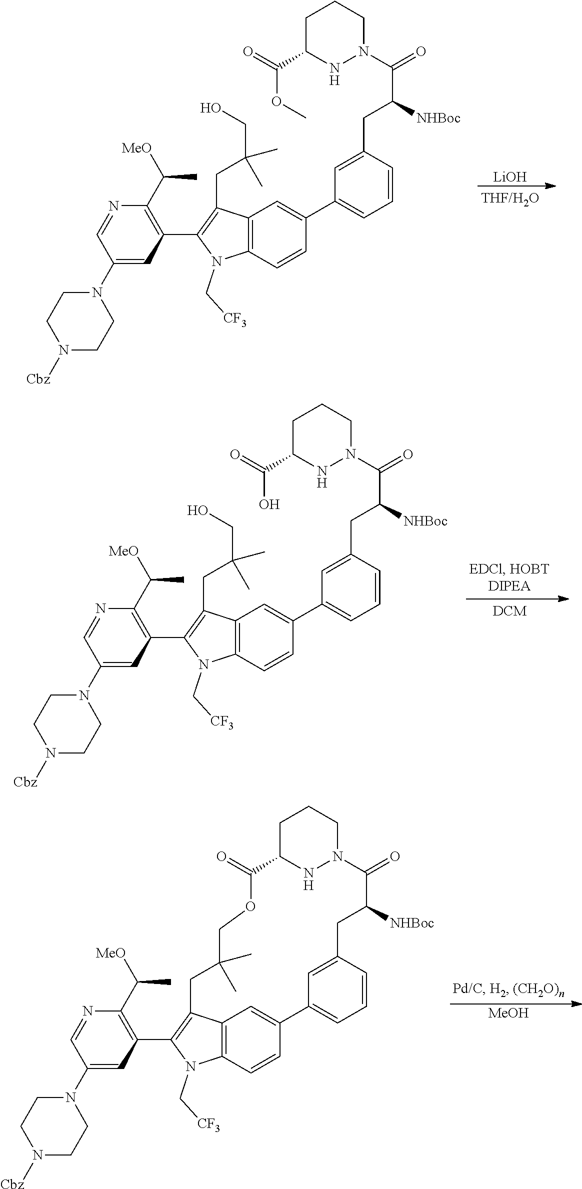

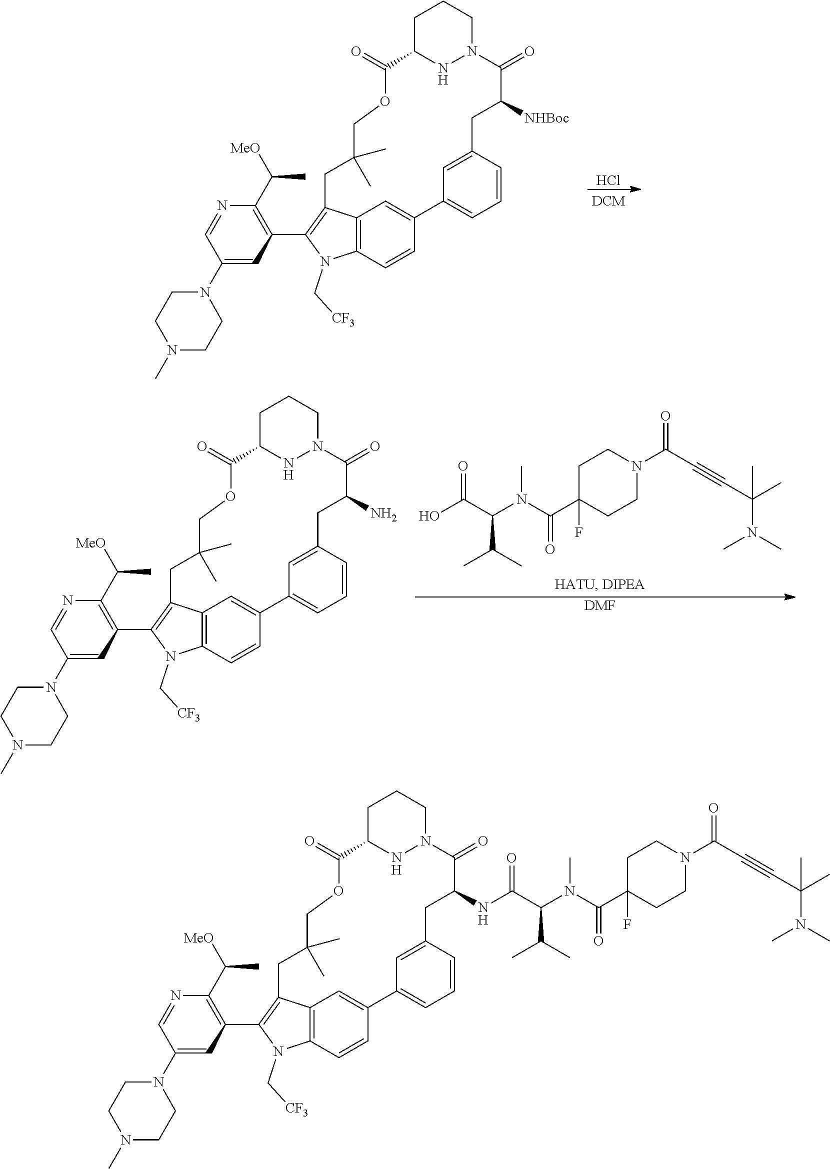

Step 1. To a solution of(S)-3-bromo-5-iodo-2-(1-methoxyethyl) pyridine (15 g, 43.86 mmol), and benzyl piperazine-1-carboxylate (8.7 g, 39.48 mmol) in toluene (150 mL) at 0° C., were added cesium carbonate (71.46 g, 219.32 mmol), BINAP (0.55 g, 0.88 mmol) and palladium acetate (0.49 g, 2.19 mmol) in portions. The reaction mixture was stirred at 90° C. for 12 h under an argon atmosphere. The resulting mixture was cooled down to room temperature, filtered and the filter cake was washed with EtOAc (150 mL×3). The filtrate was concentrated under reduced pressure. The residue was purified by silica gel column chromatography to afford benzyl(S)-4-(5-bromo-6-(1-methoxyethyl) pyridin-3-yl) piperazine-1-carboxylate (16 g, 84% yield) as solid. LCMS (ESI): m/z [M+H] calc'd for C₄₄H₅₈N₆O₇ 433.1; found 434.0.

Step 2. To a stirred solution of benzyl(S)-4-(5-bromo-6-(1-methoxyethyl) pyridin-3-yl) piperazine-1-carboxylate (22.7 g, 52.26 mmol), bis(pinacolato)diboron (19.91 g, 78.4 mmol) in toluene (230 mL) at 0° C., were added potassium acetate (12.82 g, 130.66 mmol) and Pd(dppf)Cl₂: DCM (4.26 g, 5.23 mmol) in portions. The reaction mixture was stirred at 90° C. for 6 h under an argon atmosphere. The resulting mixture was filtered and the filter cake was washed with EtOAc (200 mL×3). The filtrate was concentrated under reduced pressure. The residue was purified by silica gel column chromatography to afford benzyl(S)-4-(6-(1-methoxyethyl)-5-(4,4,5,5-tetramethyl-1,3,2-dioxaborolan-2-yl) pyridin-3-yl) piperazine-1-carboxylate (14.7 g, 58% yield) as solid. LCMS (ESI): m/z [M+H] calc'd for C₂₆H₃₆BN₃O₅ 481.3; found 482.3.

Step 3. To a stirred solution of 5-bromo-3-(3-((tert-butyldiphenylsilyl)oxy)-2,2-dimethylpropyl)-2-iodo-1H-indole (17.46 g, 27 mmol) in 1,4-dioxane (150 mL) and H₂O (30 mL) at 0° C., were added potassium carbonate (9.33 g, 67.51 mmol) and Pd(dppf)Cl₂ DCM (2.2 g, 2.7 mmol) in portions, followed by benzyl(S)-4-(6-(1-methoxyethyl)-5-(4, 4, 5, 5-tetramethyl-1, 3, 2-dioxaborolan-2-yl) pyridin-3-yl) piperazine-1-carboxylate (13 g, 27 mmol). The reaction mixture was stirred at 70° C. for 12 h under an argon atmosphere. The resulting mixture was cooled to room temperature and quenched with H₂O, then extracted with EtOAc (200 mL×3). The organic phase was dried over anhydrous sodium sulfate and concentrated under reduced pressure. The residue was purified by silica gel column chromatography to afford benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (20 g, 84.7% yield) as solid. LCMS (ESI): m/z [M+H] calc'd for C₄₉H₅₇BN₄O₄Si 873.2; found 873.3.

Step 4. To a mixture of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (19 g, 21.74 mmol) and Cs₂CO₃ (49.58 g, 152.17 mmol) in DMF (190 mL) at 0° C. under argon atmosphere, was dropwise added 2,2,2-trifluoroethyl trifluoromethanesulfonate (50.46 g, 217.39 mmol). The reaction mixture was stirred at room temperature for 12 h under an argon atmosphere, then quenched with H₂O, extracted with EtOAc (200 mL×3). The combined organic layers were washed with brine (200 mL×3), dried over anhydrous sodium sulfate and concentrated under reduced pressure. The residue was purified by silica gel column chromatography to afford benzyl(S)-4-(5-(5-bromo-3-(3-((tert-butyldiphenylsilyl)oxy)-2,2-dimethylpropyl)-1-(2,2,2-trifluoroethyl)-1H-indol-2-yl)-6-(1-methoxyethyl) pyridin-3-yl) piperazine-1-carboxylate (17.6 g, 84.7% yield) as solid. LCMS (ESI): m/z [M+H] calc'd for C₅₁H₅₈BF₃N₄O₄Si 954.2; found 955.3.

Step 5. To a solution of benzyl(S)-4-(5-(5-bromo-3-(3-((tert-butyldiphenylsilyl)oxy)-2,2-dimethylpropyl)-1-(2,2,2-trifluoroethyl)-1H-indol-2-yl)-6-(1-methoxyethyl) pyridin-3-yl) piperazine-1-carboxylate (18 g, 18.83 mmol), was added TBAF in THF (180.0 mL) at 0° C. The reaction mixture was stirred at 40° C. for 12 h under an argon atmosphere, then quenched with cold H₂O. The resulting mixture was extracted with EtOAc (200 mL×3). The combined organic layers were washed with brine (200 mL×3), dried over anhydrous sodium sulfate and concentrated under reduced pressure. The residue was purified by silica gel column chromatography to afford benzyl (S)-4-(5-(5-bromo-3-(3-hydroxy-2,2-dimethylpropyl)-1-(2,2,2-trifluoroethyl)-1H-indol-2-yl)-6-(1-methoxyethyl) pyridin-3-yl) piperazine-1-carboxylate (7.8 g, 57.7% yield) as solid. LCMS (ESI): m/z [M+H] calc'd for C₃₅H₄₀BrF₃N₄O₄ 716.2; found 717.1.

Step 6. A solution of benzyl(S)-4-(5-(5-bromo-3-(3-hydroxy-2,2-dimethylpropyl)-1-(2,2,2-trifluoroethyl)-1H-indol-2-yl)-6-(1-methoxyethyl) pyridin-3-yl) piperazine-1-carboxylate (1 g, 1.39 mmol) in 1,4-dioxane (10 mL) and H₂O (2 mL), was added methyl(S)-1-((S)-2-((tert-butoxycarbonyl) amino)-3-(3-(4,4,5,5-tetramethyl-1,3,2-dioxaborolan-2-yl)phenyl) propanoyl) hexahydropyridazine-3-carboxylate (1.08 g, 2.09 mmol), potassium carbonate (481.47 mg, 3.48 mmol) and Pd(dtbpf)Cl₂ (181.64 mg, 0.28 mmol) in portions at 0° C. The reaction mixture was stirred at 70° C. for 3 h under an argon atmosphere. The resulting mixture was cooled to room temperature, then quenched with H₂O and extracted with EtOAc (50 mL×3). The combined organic layers were washed with brine (20 mL×3), dried over anhydrous sodium sulfate and concentrated under reduced pressure. The residue was purified by silica gel chromatography to afford methyl(S)-1-((S)-3-(3-(2-(5-(4-((benzyloxy) carbonyl) piperazin-1-yl)-2-((S)-1-methoxyethyl) pyridin-3-yl)-3-(3-hydroxy-2,2-dimethylpropyl)-1-(2,2,2-trifluoroethyl)-1H-indol-5-yl)phenyl)-2-((tert-butoxycarbonyl) amino) propanoyl) hexahydropyridazine-3-carboxylate (1.1 g, 77% yield) as solid. LCMS (ESI): m/z [M+H] calc'd for C₅₅H₆₈F₃N₇O₉ 1027.5; found 1028.3.

Step 7. To a solution of methyl(S)-1-((S)-3-(3-(2-(5-(4-((benzyloxy) carbonyl) piperazin-1-yl)-2-((S)-1-methoxyethyl) pyridin-3-yl)-3-(3-hydroxy-2,2-dimethylpropyl)-1-(2,2,2-trifluoroethyl)-1H-indol-5-yl)phenyl)-2-((tert-butoxycarbonyl) amino) propanoyl) hexahydropyridazine-3-carboxylate (1.1 g, 1.07 mmol) in THF (8 mL) and H₂O (2 mL) at 0° C., was dropwise added LiOH (2.2 mL, 1M aqueous) under an argon atmosphere. The reaction mixture was stirred for 2 h then concentrated under reduced pressure. The residue was acidified to pH 5 with citric acid (1M) and extracted with EtOAc (20 mL×3). The combined organic layers were concentrated under reduced pressure. The residue was purified by reverse phase chromatography to afford(S)-1-((S)-3-(3-(2-(5-(4-((benzyloxy) carbonyl) piperazin-1-yl)-2-((S)-1-methoxyethyl) pyridin-3-yl)-3-(3-hydroxy-2,2-dimethylpropyl)-1-(2,2,2-trifluoroethyl)-1H-indol-5-yl)phenyl)-2-((tert-butoxycarbonyl) amino) propanoyl) hexahydropyridazine-3-carboxylic acid (750 mg, 69% yield) as solid. LCMS (ESI): m/z [M+H] calc'd for C₅₄H₆₆F₃N₇O₉ 1013.5; found 1014.3.

Step 8. To a solution of(S)-1-((S)-3-(3-(2-(5-(4-((benzyloxy) carbonyl) piperazin-1-yl)-2-((S)-1-methoxyethyl) pyridin-3-yl)-3-(3-hydroxy-2,2-dimethylpropyl)-1-(2,2,2-trifluoroethyl)-1H-indol-5-yl)phenyl)-2-((tert-butoxycarbonyl) amino) propanoyl) hexahydropyridazine-3-carboxylic acid (0.75 g, 0.74 mmol) in DCM (75 mL) at 0° C., were added in portions HOBT (0.5 g, 3.7 mmol), DIPEA (3.82 g, 29.58 mmol), and EDCI (4.25 g, 22.19 mmol) at 0° C. The reaction mixture was stirred at room temperature for 12 h under an argon atmosphere. The resulting mixture was concentrated under reduced pressure and extracted with EtOAc (100 mL×3). The combined organic layers were washed with brine (50 mL×3), dried over anhydrous sodium sulfate and concentrated under reduced pressure. The residue was purified by silica gel chromatography to afford benzyl 4-(5-((6³S,4S)-4-((tert-butoxycarbonyl) amino)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (0.5 g, 67.9% yield) as solid. LCMS (ESI): m/z [M+H] calc'd for C₅₄H₆₄F₃N₇O₈ 995.5; found 996.3.

Step 9. To a mixture of benzyl 4-(5-((6³S,4S)-4-((tert-butoxycarbonyl) amino)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (500 mg, 0.5 mmol) in MeOH (15 mL) at 0° C., was added paraformaldehyde (135.64 mg, 1.5 mmol), and Pd/C (750 mg) in portions. The reaction mixture was stirred at room temperature for 12 h under a hydrogen atmosphere. The resulting mixture was filtered and the filter cake was washed with EtOAc (50 mL×5). The filtrate was concentrated under reduced pressure. The residue was purified by silica gel chromatography to afford tert-butyl ((6³S,4S)-1²-(2-((S)-1-methoxyethyl)-5-(4-methylpiperazin-1-yl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (350 mg, 79.6% yield) as an solid. LCMS (ESI): m/z [M+H] calc'd for C₄₇H₆₀F₃N₇O₈ 875.5; found 876.5.

Step 10. To a solution of tert-butyl ((6³S,4S)-1²-(2-((S)-1-methoxyethyl)-5-(4-methylpiperazin-1-yl) pyridin-3-yl)-10, 10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (300 mg, 0.34 mmol) in DCM (2 mL) at 0° C., was dropwise added HCl in 1,4-dioxane (1 mL, 4M, 4 mmol). The reaction mixture was stirred at room temperature for 2 h, then concentrated under reduced pressure to give (6³S,4S)-4-amino-12-(2-((S)-1-methoxyethyl)-5-(4-methylpiperazin-1-yl) pyridin-3-yl)-10, 10-dimethyl-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione hydrochloride (350 mg, crude) as solid. LCMS (ESI): m/z [M+H] calc'd for C₄₂H₅₂F₃N₇O₄ 775.4; found 766.4.

Step 11. To a solution of (6³S,4S)-4-amino-12-(2-((S)-1-methoxyethyl)-5-(4-methylpiperazin-1-yl) pyridin-3-yl)-10,10-dimethyl-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione hydrochloride (150 mg, 0.19 mmol) and N-(1-(4-(dimethylamino)-4-methylpent-2-ynoyl)-4-fluoropiperidine-4-carbonyl)-N-methyl-L-valine (154 mg, 0.39 mmol) in DMF (2 mL) at 0° C., was dropwise added a mixture of DIPEA (1 g, 7.72 mmol) and HATU (110 mg, 0.29 mmol) in DMF (0.2 mL). The reaction mixture was stirred at 0° C. for 2 h under an argon atmosphere, then quenched with H₂O. The resulting mixture was extracted with EtOAc (20 mL×3). The combined organic phase was washed with brine (10 mL×3), dried over anhydrous sodium sulfate and concentrated under reduced pressure. The residue was purified by reverse phase chromatography to afford 1-(4-(dimethylamino)-4-methylpent-2-ynoyl)-4-fluoro-N-((2S)-1-(((6³S,4S)-1²-(2-((S)-1-methoxyethyl)-5-(4-methylpiperazin-1-yl) pyridin-3-yl)-10, 10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperidine-4-carboxamide (39.5 mg, 17% yield) as solid. ¹H NMR (400 MHZ, DMSO-d₆) δ 8.48 (d, J=2.9 Hz, 1H), 8.32 (t, J=7.3 Hz, 1H), 7.97 (s, 1H), 7.82-7.69 (m, 3H), 7.66 (t, J=7.8 Hz, 1H), 7.33-7.07 (m, 3H), 5.50 (dd, J=16.7, 8.6 Hz, 1H), 5.33 (t, J=9.2 Hz, 1H), 5.16 (d, J=12.2 Hz, 1H), 4.94-4.80 (m, 1H), 4.64 (d, J=10.8 Hz, 1H), 4.33-4.16 (m, 3H), 4.12-4.02 (m, 2H), 3.71-3.50 (m, 3H), 3.25 (s, 3H), 3.21-3.16 (m, 3H), 3.14-3.05 (m, 1H), 2.96 (t, J=4.7 Hz, 4H), 2.84 (s, 1H), 2.82-2.72 (m, 2H), 2.59-2.53 (m, 1H), 2.47-2.40 (m, 4H), 2.22 (d, J=2.9 Hz, 9H), 2.18-2.12 (m, 2H), 2.11-1.99 (m, 3H), 1.87-1.78 (m, 1H), 1.74-1.62 (m, 1H), 1.59-1.48 (m, 1H), 1.40-1.32 (m, 9H), 1.01 (t, J=7.7 Hz, 1H), 0.89 (s, 5H), 0.83 (d, J=6.3 Hz, 1H), 0.77 (d, J=6.6 Hz, 2H), 0.38 (s, 3H). LCMS (ESI): m/z [M+H] calc'd for C₄₄H₅₈N₆O₇ 1154.6; found 1155.7.

### Example A735. Synthesis of 2-acryloyl-N-((2S)-1-(((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-N-methyl-5-oxa-2,9-diazaspiro[3.5]nonane-9-carboxamide

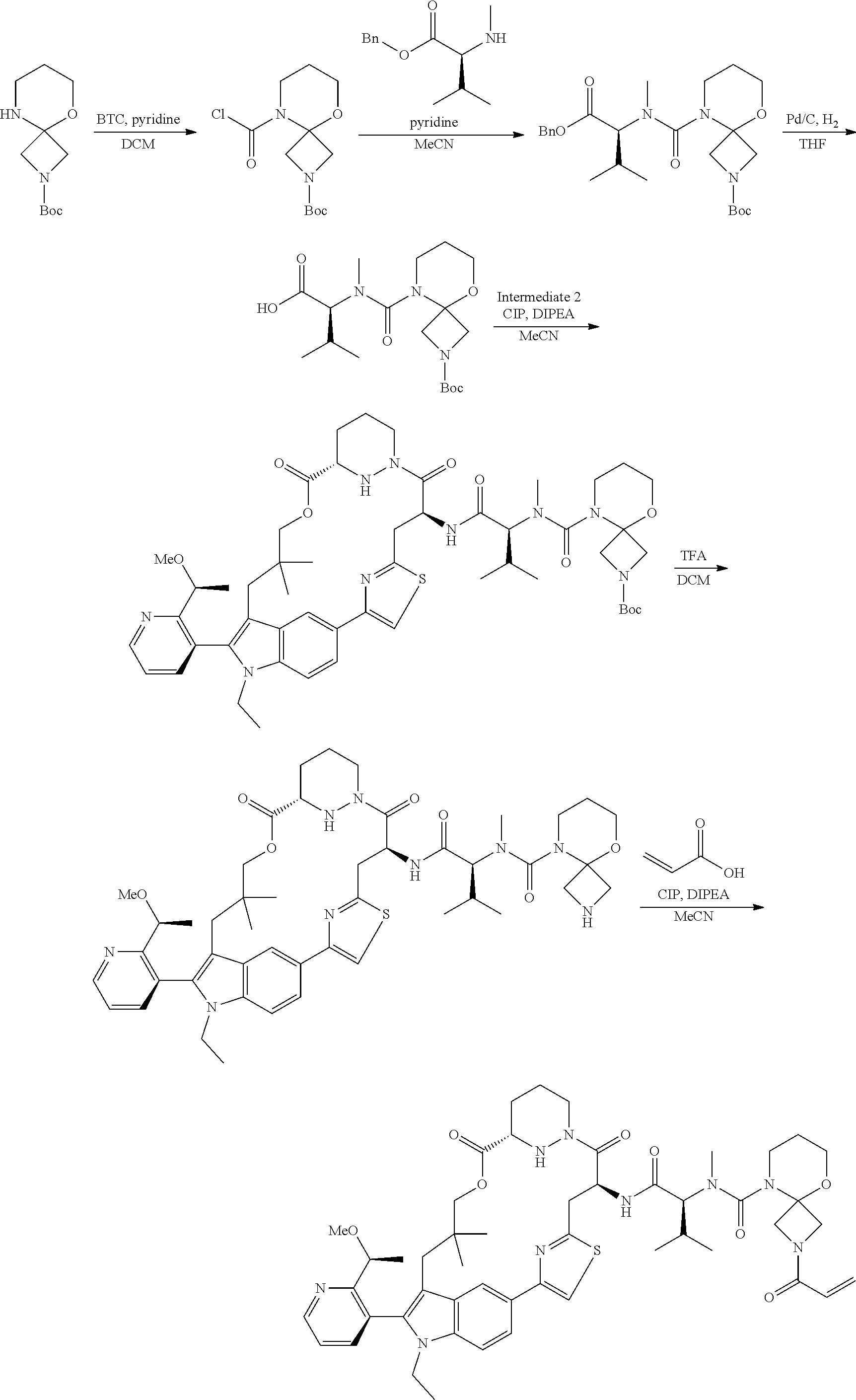

Step 1. To a stirred mixture of BTC (425.2 mg, 1.448 mmol) in DCM (10 mL) was added dropwise pyridine (1.04 g, 13.16 mmol) and tert-butyl 5-oxa-2,9-diazaspiro[3.5]nonane-2-carboxylate (1 g, 4.39 mmol), the reaction mixture was stirred at room temperature for 2 h. The resulting mixture was concentrated under reduced pressure to give crude tert-butyl 9-(chlorocarbonyl)-5-oxa-2,9-diazaspiro[3.5]nonane-2-carboxylate.

Step 2. To a stirred solution of tert-butyl 9-(chlorocarbonyl)-5-oxa-2,9-diazaspiro[3.5]nonane-2-carboxylate (2.5 g, crude) in MeCN (20 mL) were added dropwise pyridine (1.04 g, 13.16 mmol) and benzyl (2S)-3-methyl-2-(methylamino) butanoate (970.66 mg, 4.38 mmol) at room temperature. The reaction mixture was stirred at 80° C. for 12 h and concentrated under reduced pressure. The residue was purified by silica gel column chromatography to afford tert-butyl(S)-9-((1-(benzyloxy)-3-methyl-1-oxobutan-2-yl) (methyl) carbamoyl)-5-oxa-2,9-diazaspiro[3.5]nonane-2-carboxylate (783 mg, 37.6% yield, two steps) as an oil. LCMS (ESI): m/z [M+H] calc'd for C₂₅H₃₇N₃O₆ 475.3; found 476.3.

Step 3. A solution of tert-butyl(S)-9-((1-(benzyloxy)-3-methyl-1-oxobutan-2-yl) (methyl) carbamoyl)-5-oxa-2,9-diazaspiro[3.5]nonane-2-carboxylate (783 mg, 1.65 mmol) and 10 wt % palladium on carbon (226.29 mg) in THF (10 mL) was stirred for 2 h at 50° C. under a hydrogen atmosphere. The resulting mixture was cooled to room temperature, filtered and the filter cake was washed with MeCN (10 mL×3). The filtrate was concentrated under reduced pressure to give N-(2-(tert-butoxycarbonyl)-5-oxa-2,9-diazaspiro[3.5]nonane-9-carbonyl)-N-methyl-L-valine (591 mg, 98.8% yield) as solid. LCMS (ESI): m/z [M+H] calc'd for C₁₈H₃₁N₃O₆ 385.2; found 386.3.

Step 4. To a stirred solution of intermediate 2 (731 mg, 1.16 mmol) and DIPEA (2.25 g, 17.38 mmol) in MeCN (50 mL) was added CIP (644.31 mg, 2.32 mmol) and N-(2-(tert-butoxycarbonyl)-5-oxa-2,9-diazaspiro[3.5]nonane-9-carbonyl)-N-methyl-L-valine (446.68 mg, 1.16 mmol) at room temperature. The reaction mixture was stirred for 2 h then concentrated under reduced pressure. The residue was purified by silica gel column chromatography to afford tert-butyl 9-(((2S)-1-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl) (methyl) carbamoyl)-5-oxa-2,9-diazaspiro[3.5]nonane-2-carboxylate (752 mg, 65% yield) as solid. LCMS (ESI): m/z [M+H] calc'd for C₅₂H₇₁N₉O₉S 997.5; found 996.6.

Step 5. To a stirred solution of tert-butyl 9-(((2S)-1-(((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl) (methyl) carbamoyl)-5-oxa-2,9-diazaspiro[3.5]nonane-2-carboxylate (752 mg, 0.75 mmol) in DCM (40 mL) was added TFA (10 mL) in portions at room temperature. The reaction mixture was stirred for 2 h, then concentrated under reduced pressure. To the residue was added saturated aqueous sodium bicarbonate (100 mL) and DCM (100 mL). The aqueous layer was separated and extracted with DCM (100 mL×2). The combined organic phase was dried over anhydrous sodium sulfate, filtrated and concentrated under reduced pressure to afford N-((2S)-1-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-N-methyl-5-oxa-2,9-diazaspiro[3.5]nonane-9-carboxamide (587 mg, 87% yield) as solid. LCMS (ESI): m/z [M+H] calc'd for C₄₇H₆₃N₉O₇S 897.5; found 898.4.

Step 6. A stirred solution of N-((2S)-1-(((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-N-methyl-5-oxa-2,9-diazaspiro[3.5]nonane-9-carboxamide (586 mg, 0.65 mmol) in MeCN (10 mL) was added acrylic acid (47 mg, 0.65 mmol), DIPEA (421 mg, 3.26 mmol), CIP (362 mg, 1.3 mmol). The reaction mixture was stirred for 12 h and concentrated under reduced pressure. The residue was purified by reverse phase chromatography to afford 2-acryloyl-N-((2S)-1-(((6³S,4S,Z)-1¹-ethyl-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) amino)-3-methyl-1-oxobutan-2-yl)-N-methyl-5-oxa-2,9-diazaspiro[3.5]nonane-9-carboxamide (194 mg, 27% yield) as a solid. ¹H NMR (400 MHZ, DMSO-d₆) δ 8.78 (dd, J=4.8, 1.7 Hz, 1H), 8.48 (d, J=1.6 Hz, 2H), 7.85-7.65 (m, 3H), 7.65-7.42 (m, 2H), 6.30 (mm, 1H), 6.10 (m, J=17.0, 1H), 5.78-5.50 (m, J=10.3, 2H), 5.10 (dd, 1H), 4.40-3.80 (m, 14H), 3.60-3.10 (m, 10H), 2.94 (d, J=14.5 Hz, 1H), 2.85 (s, 4H), 2.42 (dd, 1H), 2.07 (dd, 2H), 1.80 (s, 2H), 1.55 (s, 3H), 1.32 (d, 3H), 0.95-0.75 (m, 12H), 0.33 (s, 3H). LCMS (ESI): m/z [M+H] calc'd for C₅₀H₆₅N₉O₈S 951.5; found 952.6.

### Example A720. Synthesis of (2R)-2-(((1-(4-(dimethylamino)-4-methylpent-2-ynoyl) azetidin-3-yl)oxy) methyl)-N-((6³S,4S,Z)-1¹-ethyl-1²-(2-((S)-1-methoxyethyl) pyridin-3-yl)-21,10,10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide

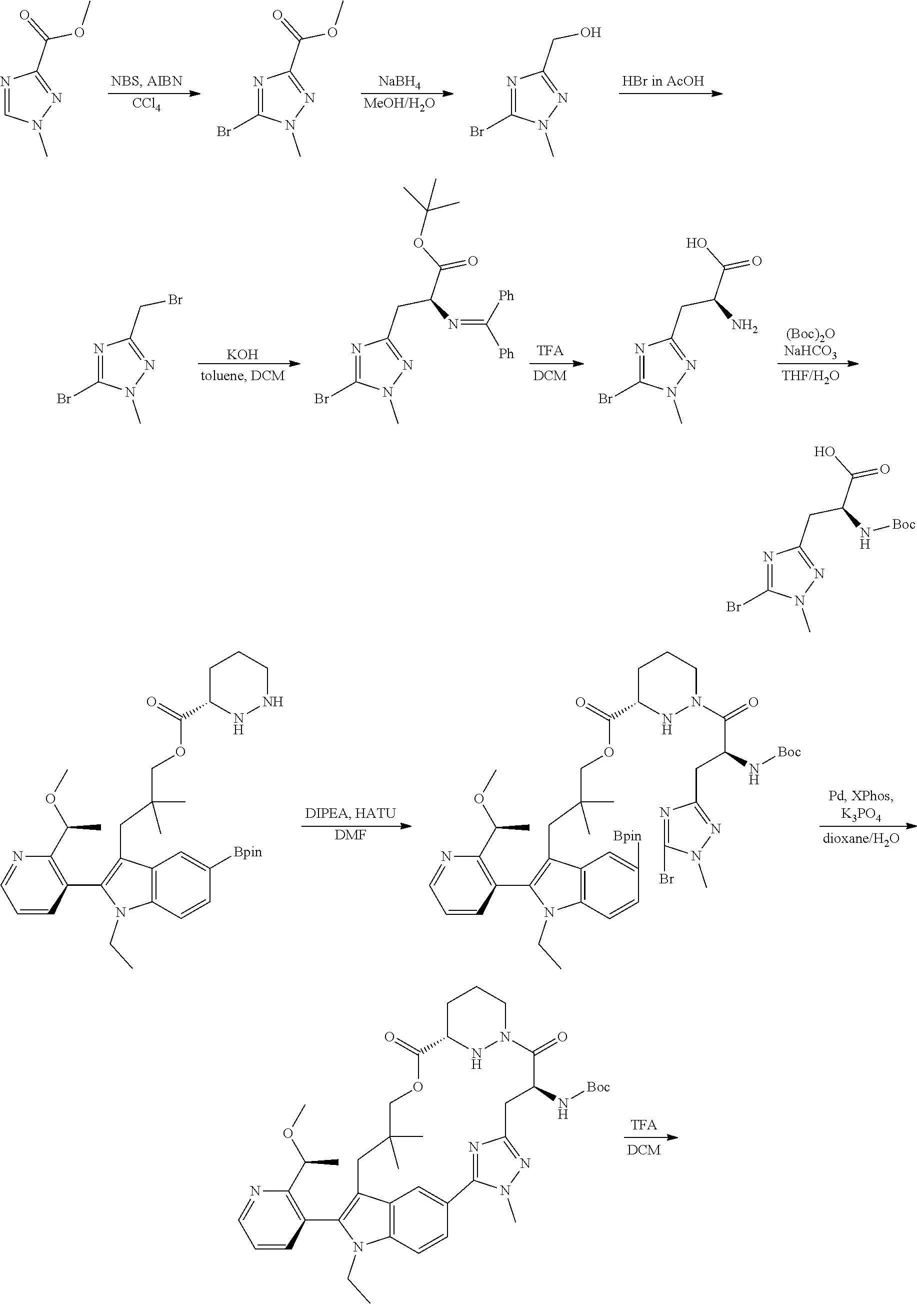

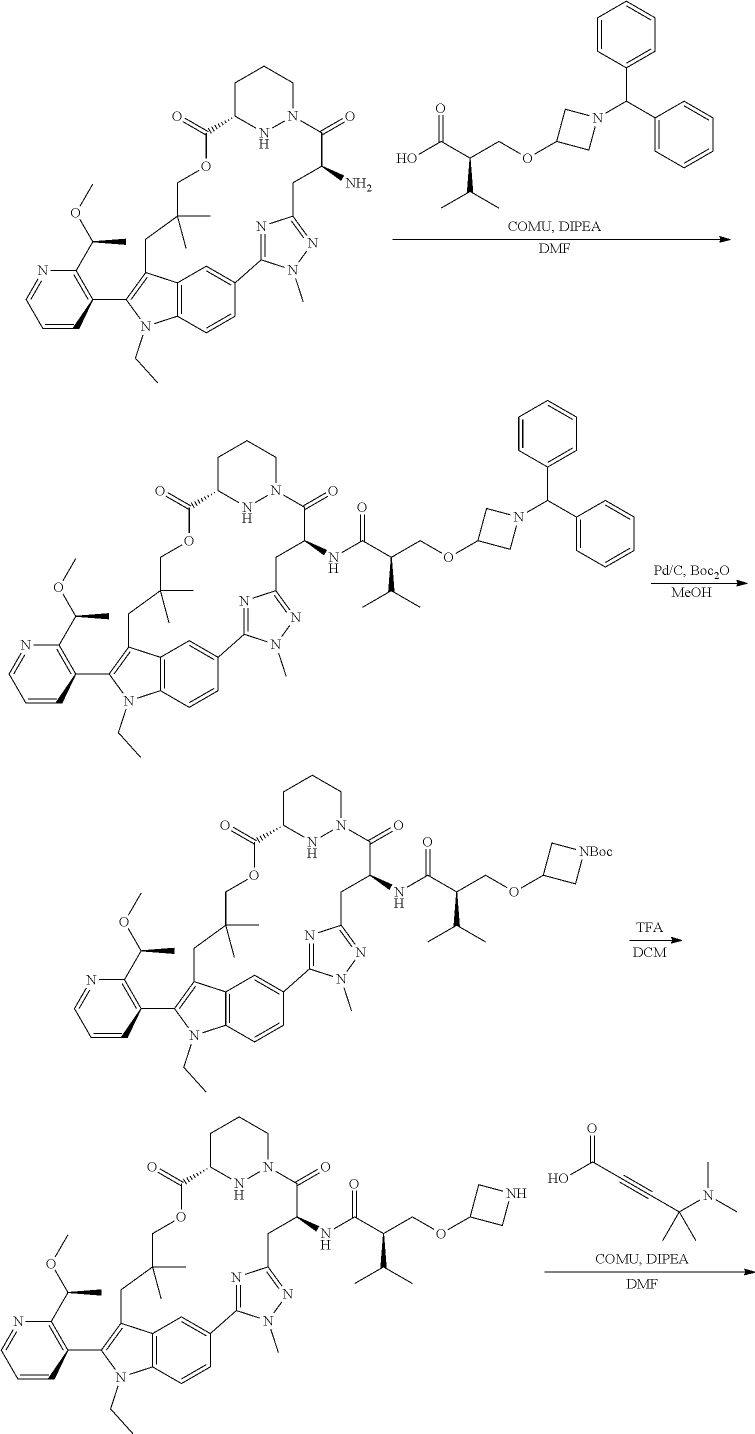

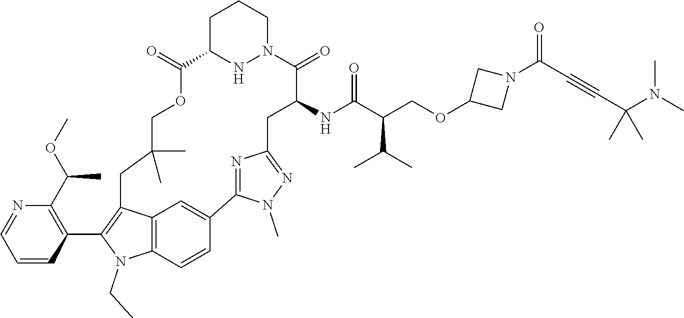

Step 1. To a stirred solution of methyl 1-methyl-1,2,4-triazole-3-carboxylate (7.0 g, 49.60 mmol) in CCl₄ (70. mL) was added NBS (13.24 g, 74.40 mmol) and AIBN (11.40 g, 69.44 mmol) in portions at 25° C. under an argon atmosphere. The resulting mixture was stirred for 24 h at 80° C. The resulting mixture was filtered, the filtrate was cooled to 20° C. and kept at 20° C. for 30 min. The resulting mixture was filtered. The filter cake was washed with H₂O (3×50 mL) and pet. ether (3×100 mL). The filter cake was dried under reduced pressure. This resulted in methyl 5-bromo-1-methyl-1,2,4-triazole-3-carboxylate (10 g, crude) as a light yellow solid. LCMS (ESI): m/z [M+H] calc'd for C₅H₆BrN₃O₂ 219.0; found 219.9.

Step 2. To a stirred solution of methyl 5-bromo-1-methyl-1,2,4-triazole-3-carboxylate (10.0 g, 45.50 mmol) in MeOH (150.0 mL) and H₂O (30.0 mL) was added NaBH₄ (6.88 g, 181.80 mmol) in portions at −5° C. under a nitrogen atmosphere. The resulting mixture was stirred for 2 h at 0˜10° C. Desired product could be detected by LCMS. The reaction was quenched with brine (100 mL) at 0° C. The resulting mixture was extracted with pet. ether (100 mL). The aqueous layer was separated and filtered. The filter cake was washed with MeOH (2×50 mL). The filtrate was concentrated under reduced pressure to afford (5-bromo-1-methyl-1,2,4-triazol-3-yl) methanol (6 g, crude) as a light yellow solid. LCMS (ESI): m/z [M+H] calc'd for C₄H₆BrN₃O 191.98; found 192.0.

Step 3. A solution of (5-bromo-1-methyl-1,2,4-triazol-3-yl) methanol (6.0 g) and HBr in AcOH (144.0 mL) was stirred for overnight at 80° C. . . . The mixture was neutralized to pH 9 with saturated NaHCO₃(aq.). The resulting mixture was extracted with EtOAc (3×60 mL). The combined organic layers were washed with brine (3×100 mL), dried over anhydrous Na₂SO₄. After filtration, the filtrate was concentrated under reduced pressure. This resulted in 5-bromo-3-(bromomethyl)-1-methyl-1,2,4-triazole (6 g, crude) as a white solid. LCMS (ESI): m/z [M+H] calc'd for C₄H₅Br₂N₃ 253.89; found 253.8.

Step 4. To a stirred mixture of 5-bromo-3-(bromomethyl)-1-methyl-1,2,4-triazole (6.0 g, 23.54 mmol) and tert-butyl 2-[(diphenylmethylidene) amino]acetate (6.95 g, 23.54 mmol) in toluene (42 mL) and DCM (18.0 mL) was added (2R,4R,5S)-1-(anthracen-9-ylmethyl)-5-ethenyl-2-[(S)-(prop-2-en-1-yloxy) (quinolin-4-yl)methyl]-1-azabicyclo[2.2.2]octan-1-ium bromide (1.43 g, 2.35 mmol) in portions at 0° C. under argon atmosphere. The resulting mixture was stirred and KOH (60 mL) in H₂O was added. The resulting mixture was stirred for 24 h at −10° C. under an argon atmosphere. Desired product could be detected by LCMS. The reaction was quenched with sat. NH₄Cl (aq.) at 0° C. The resulting mixture was extracted with EtOAc (3×100 mL). The combined organic layers were washed with brine (1×200 mL), dried over anhydrous Na₂SO₄. After filtration, the filtrate was concentrated under reduced pressure. The residue was purified by Prep-TLC to afford tert-butyl (2S)-3-(5-bromo-1-methyl-1,2,4-triazol-3-yl)-2-[(diphenylmethylidene) amino]propanoate (5 g, 38.6% yield) as a yellow oil. LCMS (ESI): m/z [M+H] calc'd for C₂₁H₂₁BrN₄O₂ 469.12; found 469.1.

Step 5. To a stirred solution of tert-butyl (2S)-3-(5-bromo-1-methyl-1,2,4-triazol-3-yl)-2-[(diphenylmethylidene) amino]propanoate (5.0 g, 10.65 mmol) in DCM (50.0 mL) was added TFA (25.0 mL) dropwise at 0° C. under argon atmosphere. The resulting mixture was stirred for 16 h at room temperature under an argon atmosphere. The resulting mixture was concentrated under reduced pressure to afford (2S)-2-amino-3-(5-bromo-1-methyl-1,2,4-triazol-3-yl) propanoic acid (6 g, crude) as a brown oil. LCMS (ESI): m/z [M+H] calc'd for C₆H₉BrN₄O₂ 249.00; found 249.0.

Step 6. To a stirred solution of (2S)-2-amino-3-(5-bromo-1-methyl-1,2,4-triazol-3-yl) propanoic acid (6.0 g, 24.09 mmol) in THF (36.0 mL) was added NaHCO₃ (10.14 g, 120.69 mmol), Boc₂O (7.89 g, 36.14 mmol) in portions at 0° C. under an argon atmosphere. The resulting mixture was stirred for 16 h at room temperature. Desired product could be detected by LCMS. The resulting mixture was concentrated under reduced pressure. The mixture was purified by reverse phase chromatography to afford (2S)-3-(5-bromo-1-methyl-1,2,4-triazol-3-yl)-2-[(tert-butoxycarbonyl) amino]propanoic acid (3 g, 33.9% yield) as a white solid. LCMS (ESI): m/z [M+H] calc'd for C₁₁H₁₇BrN₄O₄ 349.05; found 349.0.

Step 7. To a stirred solution of 2-[(2M)-1-ethyl-2-[2-[(1S)-1-methoxyethyl]pyridin-3-yl]-5-(4,4,5,5-tetramethyl-1,3,2-dioxaborolan-2-yl) indol-3-yl]methyl]-2-methylpropyl (3S)-1,2-diazinane-3-carboxylate (1.0 g, 1.65 mmol) in DMF (10.0 mL) was added DIPEA (4.28 g, 33.08 mmol), (2S)-3-(5-bromo-1-methyl-1,2,4-triazol-3-yl)-2-[(tert-butoxycarbonyl) amino]propanoic acid (0.69 g, 1.98 mmol) and HATU (0.75 g, 1.99 mmol) in portions at 0° C. The resulting mixture was stirred for 2 h at 20° C. under an argon atmosphere. Desired product could be detected by LCMS. The resulting mixture was quenched with H₂O (100 mL) and extracted with EtOAc (3×30 mL). The combined organic layers were washed with brine (3×100 mL), dried over anhydrous Na₂SO₄. After filtration, the filtrate was concentrated under reduced pressure. The residue was purified by reverse phase chromatography to afford 2-[(2M)-1-ethyl-2-[2-[(1S)-1-methoxyethyl]pyridin-3-yl]-5-(4,4,5,5-tetramethyl-1,3,2-dioxaborolan-2-yl) indol-3-yl]methyl]-2-methylpropyl (3S)-1-[(2S)-3-(5-bromo-1-methyl-1,2,4-triazol-3-yl)-2-[(tert-butoxycarbonyl) amino]propanoyl]-1,2-diazinane-3-carboxylate (800 mg, 46.5% yield) as a light yellow solid. LCMS (ESI): m/z [M+H] calc'd for C₄₅H₆₄BBrN₈O₈ 935.42; found 935.2.

Step 8. To a stirred solution of 2-[(2M)-1-ethyl-2-[2-[(1S)-1-methoxyethyl]pyridin-3-yl]-5-(4,4,5,5-tetramethyl-1,3,2-dioxaborolan-2-yl) indol-3-yl]methyl]-2-methylpropyl (3S)-1-[(2S)-3-(5-bromo-1-methyl-1,2,4-triazol-3-yl)-2-[(tert-butoxycarbonyl) amino]propanoyl]-1,2-diazinane-3-carboxylate (800.0 mg, 0.86 mmol) in dioxane (10.0 mL) were added K₃PO₄ (0.45 g, 2.12 mmol), XPhos (122.26 mg, 0.27 mmol), XPhos Pd G3 (0.22 g, 0.27 mmol) and H₂O (2.0 mL) at room temperature. The resulting mixture was stirred for 3 h at 75° C. under an argon atmosphere. Desired product could be detected by LCMS. The resulting mixture was extracted with EtOAc (3×100 mL). The combined organic layers were washed with brine (3×60 mL), dried over anhydrous Na₂SO₄. After filtration, the filtrate was concentrated under reduced pressure. The residue was purified by reverse phase chromatography to afford tert-butyl ((6³S,4S,Z)-1¹-ethyl-1²-(2-((S)-1-methoxyethyl) pyridin-3-yl)-21, 10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl) carbamate (400 mg, 56.8% yield) as a light yellow solid. LCMS (ESI): m/z [M+H] calc'd for C₃₉H₅₂N₈O₆ 729.41; found 729.3.

Step 9. To a solution of tert-butyl ((6³S,4S,Z)-1¹-ethyl-1²-(2-((S)-1-methoxyethyl) pyridin-3-yl)-21,10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl) carbamate (400.0 mg, 0.56 mmol) in DCM (1 mL) was added TFA (0.5 mL). The reaction was stirred for 1 h at room temperature under an argon atmosphere. After concentration, the mixture was neutralized to pH 8 with saturated NaHCO₃(aq., 20 mL). The mixture was extracted with DCM (3×20 mL). The organic layers were dried over Na₂SO₄ and concentrated to afford (6³S,4S,Z)-4-amino-1¹-ethyl-1²-(2-((S)-1-methoxyethyl) pyridin-3-yl)-21,10, 10-trimethyl-6¹,6²,6³,6⁴,6⁵,6⁶-hexahydro-11H,21H-8-oxa-1 (5,3)-indola-6 (1,3)-pyridazina-2 (5,3)-triazolacycloundecaphane-5,7-dione (500 mg, crude) as a light yellow solid. ESI-MS m/z=629.3 [M+H]⁺; Calculated MW: 628.3. LCMS (ESI): m/z [M+H] calc'd for C₃₄H₄₄N₈O₄ 629.36; found 629.3.

Step 10. To a stirred solution of (6³S,4S,Z)-4-amino-1¹-ethyl-1²-(2-((S)-1-methoxyethyl) pyridin-3-yl)-21, 10, 10-trimethyl-6¹,6²,6³,6⁴,6⁵,6⁶-hexahydro-11H,21H-8-oxa-1 (5,3)-indola-6 (1,3)-pyridazina-2 (5,3)-triazolacycloundecaphane-5,7-dione (170.0 mg, 0.27 mmol) and (R)-2-(((1-benzhydrylazetidin-3-yl)oxy) methyl)-3-methylbutanoic acid (114.68 mg, 0.32 mmol) in DMF (5 mL) were added DIPEA (698.86 mg, 5.41 mmol) and HATU (123.36 mg, 0.32 mmol) dropwise at 0° C. under an air atmosphere. The resulting mixture was stirred for 2 h at 0° C. The resulting mixture was diluted with 25 mL H₂O. The resulting mixture was extracted with EtOAc (3×25 mL). The combined organic layers were washed with brine (3×25 mL), dried over anhydrous Na₂SO₄. After filtration, the filtrate was concentrated under reduced pressure to afford (2R)-2-(((1-benzhydrylazetidin-3-yl)oxy) methyl)-N-((6³S,4S,Z)-1¹-ethyl-1²-(2-((S)-1-methoxyethyl) pyridin-3-yl)-21,10,10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide (180 mg, crude) as an off-white oil. LCMS (ESI): m/z [M+H] calc'd for C₅₆H₆₉N₉O₆ 964.54; found 964.4.

Step 11. To a stirred solution of (2R)-2-(((1-benzhydrylazetidin-3-yl)oxy) methyl)-N-((6³S,4S,Z)-1¹-ethyl-1²-(2-((S)-1-methoxyethyl) pyridin-3-yl)-21,10,10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide (180.0 mg, 0.19 mmol) and Pd/C (90.0 mg, 0.85 mmol) in MeOH (10 mL) was added Boc₂O (81.48 mg, 0.37 mmol) at room temperature under a hydrogen atmosphere. The resulting mixture was stirred overnight at room temperature. The resulting mixture was filtered, the filter cake was washed with MeOH (3×10 mL). The filtrate was concentrated under reduced pressure to afford tert-butyl 3-((2R)-2-(((6³S,4S,Z)-1¹-ethyl-1²-(2-((S)-1-methoxyethyl) pyridin-3-yl)-21, 10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl) carbamoyl)-3-methylbutoxy) azetidine-1-carboxylate (80 mg, 47.7% yield) as an off-white solid. LCMS (ESI): m/z [M+H] calc'd for C₄₈H₆₇N₉O₈ 898.52; found 898.4.

Step 12. To a stirred solution of tert-butyl 3-((2R)-2-(((6³S,4S,Z)-1¹-ethyl-1²-(2-((S)-1-methoxyethyl) pyridin-3-yl)-21, 10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl) carbamoyl)-3-methylbutoxy) azetidine-1-carboxylate in DCM (2 mL) was added TFA (1.0 mL) dropwise at 0° C. under an air atmosphere. The resulting mixture was stirred for 1 h at 0° C. The resulting mixture was concentrated under reduced pressure to afford (2R)-2-((azetidin-3-yloxy) methyl)-N-((6³S,4S,Z)-11-ethyl-1²-(2-((S)-1-methoxyethyl) pyridin-3-yl)-21, 10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide (85 mg, crude) as a yellow green oil.

Step 13. To a stirred solution of (2R)-2-((azetidin-3-yloxy) methyl)-N-((6³S,4S,Z)-1¹-ethyl-12-(2-((S)-1-methoxyethyl) pyridin-3-yl)-21,10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide (80.0 mg, 0.10 mmol) and 4-(dimethylamino)-4-methylpent-2-ynoic acid (38.90 mg, 0.25 mmol) in DMF (2 mL) were added DIPEA (518.27 mg, 4.01 mmol) and COMU (51.52 mg, 0.12 mmol) in portions at 0° C. The reaction mixture was stirred under an air atmosphere for 2 h. The crude product (150 mg) was purified by reverse phase chromatography to afford (2R)-2-(((1-(4-(dimethylamino)-4-methylpent-2-ynoyl) azetidin-3-yl)oxy) methyl)-N-((6³S,4S,Z)-1¹-ethyl-1²-(2-((S)-1-methoxyethyl) pyridin-3-yl)-21, 10, 10-trimethyl-5,7-dioxo-6¹,6²,6³,6⁴,6⁵,6⁶-hexahydro-11H,21H-8-oxa-1 (5,3)-indola-6 (1,3)-pyridazina-2 (5,3)-triazolacycloundecaphane-4-yl)-3-methylbutanamide (15.3 mg, 16.3% yield) as an off-white solid. ¹H NMR (400 MHZ, DMSO-d₆) δ 8.77 (dd, J=4.8, 1.7 Hz, 1H), 8.15 (d, J=1.7 Hz, 1H), 8.07 (d, J=8.1 Hz, 1H), 7.85-7.78 (m, 1H), 7.70 (d, J=8.6 Hz, 1H), 7.58-7.48 (m, 2H), 5.82 (s, 1H), 4.95 (d, J=11.7 Hz, 1H), 4.41-4.30 (m, 5H), 4.30 (d, J=8.2 Hz, 2H), 4.25 (d, J=5.6 Hz, 4H), 4.10 (td, J=17.1, 16.1, 9.1 Hz, 2H), 3.99-3.82 (m, 3H), 3.71-3.60 (m, 1H), 3.54-3.43 (m, 3H), 3.39 (s, 2H), 3.22 (d, J=1.6 Hz, 1H), 2.92 (d, J=13.6 Hz, 1H), 2.86-2.77 (m, 2H), 2.45 (s, 6H), 2.37 (q, J=7.7 Hz, 1H), 2.17 (d, J=6.6 Hz, 2H), 2.03 (d, J=10.2 Hz, 2H), 1.78-1.66 (m, 3H), 1.47 (t, J=10.9 Hz, 6H), 1.35-1.28 (m, 12H), 0.32 (s, 3H). LCMS (ESI): m/z [M+H] calc'd for C₅₁H₇₀N₁₀O₇ 935.55; found 935.3.

### Example A692. Synthesis of (3S)-1-(4-(dimethylamino)-4-methylpent-2-ynoyl)-N-((2S)-1-(((63,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl) amino)-3-methyl-1-oxobutan-2-yl)-N-methylpyrrolidine-3-carboxamide

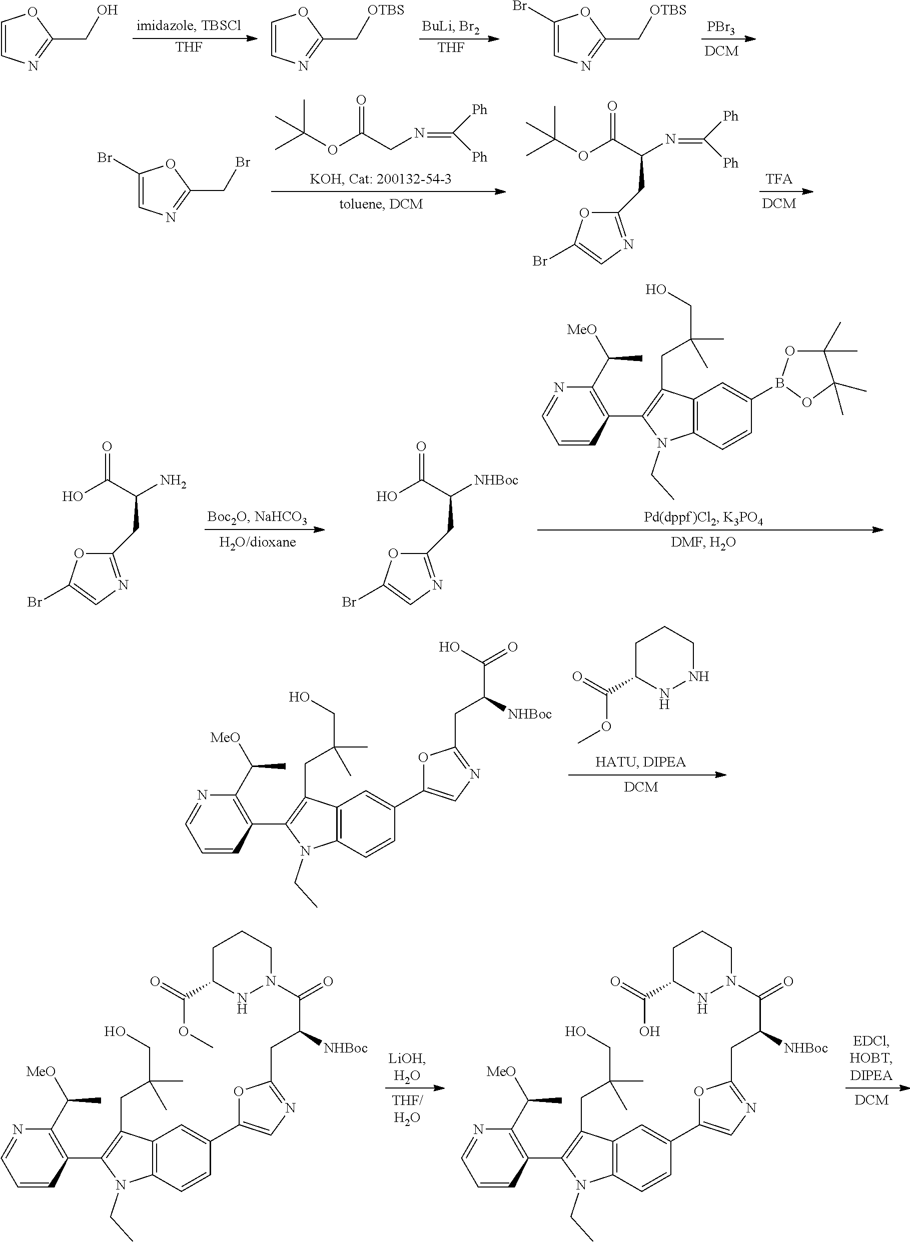

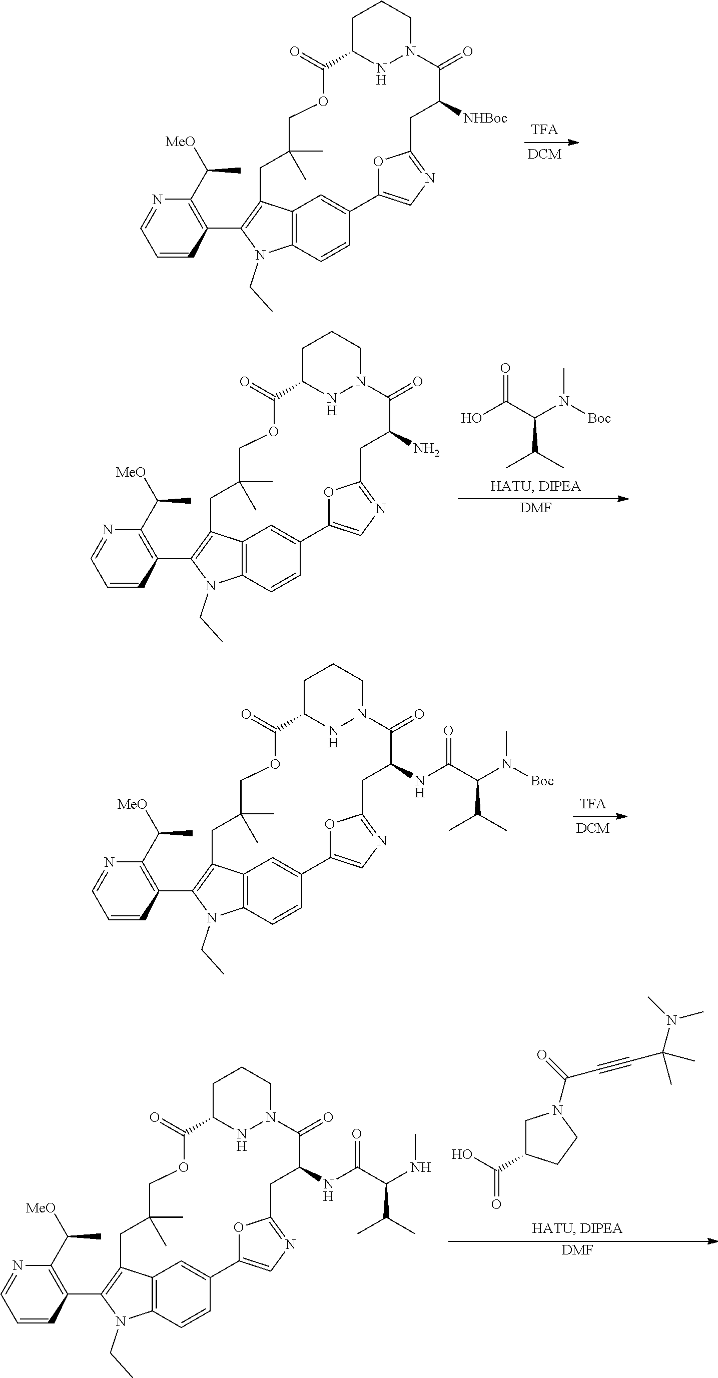

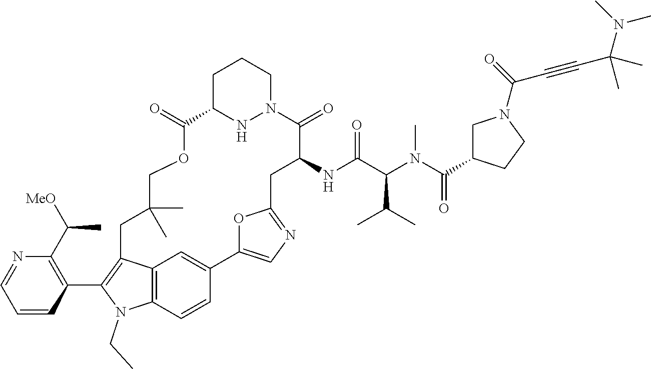

Step 1. To a solution of 1,3-oxazol-2-ylmethanol (5.0 g, 50.46 mmol) in THF (75 mL), were added imidazole (8.59 mg, 0.13 mmol), and TBSCl (11.41 mg, 0.08 mmol) at 0° C. The resulting solution was stirred for 5 h then concentrated under reduced pressure. The crude material was purified by silica gel column chromatography to afford 2-[(tert-butyldimethylsilyl)oxy]methyl]-1,3-oxazole (10 g, 92.8% yield) as colorless oil. LCMS (ESI): m/z [M+H] calc'd for C₁₀H₁₉NO₂Si 214.13; found 214.3.

Step 2. To a solution of 2-[(tert-butyldimethylsilyl)oxy]methyl]-1,3-oxazole (10.0 g, 46.87 mmol) in THF (150.0 mL, 1851.45 mmol) at −78° C. was added n-BuLi (22.4 mL, 56.25 mmol) over 10 min and stirred for 30 min at −78° C. under an argon atmosphere. Then the solution of Br₂ (3.6 mL, 70.31 mmol) in THF (10 mL) was added over 10 min to the solution at −78° C. The resulting solution was slowly warmed to room temperature and stirred for 2 h. The resulting mixture was diluted with NH₄Cl/H₂O (100 mL) and extracted with EtOAc (3×100 mL). The combined organic layers were dried over anhydrous Na₂SO₄. After filtration, the filtrate was concentrated under reduced pressure and purified by silica gel column chromatography to afford 5-bromo-2-[(tert-butyldimethylsilyl)oxy]methyl]-1,3-oxazole (5.3 g, 38.6% yield) as yellow oil. LCMS (ESI): m/z [M+H] calc'd for C₁₀H₁₈BrNO₂Si 292.04; found 292.0.

Step 3. To a solution of 5-bromo-2-[(tert-butyldimethylsilyl)oxy]methyl]-1,3-oxazole (4.0 g, 13.69 mmol) in DCM (60.0 mL) was added PBr₃ (7.41 g, 27.37 mmol) at 0° C. under an argon atmosphere. The resulting solution was stirred for 4 h then diluted with NaHCO₃/H₂O (30 mL). The mixture was extracted with EtOAc (3×40 mL). The organic layers were concentrated under reduced pressure and purified by silica gel column chromatography to afford 5-bromo-2-(bromomethyl)-1,3-oxazole (2.5 g, 75.7% yield) as yellow oil. LCMS (ESI): m/z [M+H] calc'd for C₄H₃Br₂NO 239.87; found 241.9.

Step 4. A mixture of 5-bromo-2-(bromomethyl)-1,3-oxazole (9.0 g, 37.36 mmol), Cat: 200132-54-3 (2.26 g, 3.74 mmol), DCM (45.0 mL), toluene (90.0 mL), KOH (20.96 g, 373.63 mmol), H₂O (42 mL), and tert-butyl 2-[(diphenylmethylidene) amino]acetate (13.24 g, 44.82 mmol) at 0° C. was stirred for 4 h then diluted with H₂O (30 mL). The mixture was extracted with DCM (3×40 mL). The organic layers were concentrated under reduced pressure and purified by reverse phase column chromatography to afford tert-butyl (2S)-3-(5-bromo-1,3-oxazol-2-yl)-2-[(diphenylmethylidene) amino]propanoate (4.8 g, 28.2% yield) as a yellow solid. LCMS (ESI): m/z [M+H] calc'd for C₂₃H₂₃BrN₂O₃ 455.10; found 457.1.

Step 5. A mixture of tert-butyl (2S)-3-(5-bromo-1,3-oxazol-2-yl)-2-[(diphenylmethylidene) amino]propanoate (1.20 g, 2.64 mmol), DCM (10.0 mL, 157.30 mmol), and TFA (5.0 mL, 67.32 mmol) at 0° C. was stirred for 2 h then concentrated under reduced pressure to afford(S)-3-(5-bromooxazol-2-yl)-2-((2,2,2-trifluoroacetyl)-14-azaneyl) propanoic acid (0.5 g, 81.3% yield) as a yellow solid. LCMS (ESI): m/z [M+H] calc'd for C₆H₇BrN₂O₃ 234.97; found 237.0.

Step 6. A mixture of(S)-3-(5-bromooxazol-2-yl)-2-((2,2,2-trifluoroacetyl)-14-azaneyl) propanoic acid (500.0 mg, 2.13 mmol), Boc₂O (928.56 mg, 4.26 mmol), dioxane (2.50 mL), H₂O (2.50 mL), and NaHCO₃ (714.84 mg, 8.51 mmol) at 0° C. was stirred for 3 h. The resulting solution was purified by reverse phase column chromatography to afford (2S)-3-(5-bromo-1,3-oxazol-2-yl)-2-[(tert-butoxycarbonyl) amino]propanoic acid (0.65 g, 91.1% yield) as a yellow solid. LCMS (ESI): m/z [M+H] calc'd for C₁₁H₁₅BrN₂O₅ 335.02; found 334.8.

Step 7. To a solution of (2S)-3-(5-bromo-1,3-oxazol-2-yl)-2-[(tert-butoxycarbonyl) amino]propanoic acid (500.0 mg, 1.49 mmol) and 3-[(2M)-1-ethyl-2-[2-[(1S)-1-methoxyethyl]pyridin-3-yl]-5-(4,4,5,5-tetramethyl-1,3,2-dioxaborolan-2-yl) indol-3-yl]-2,2-dimethylpropan-1-ol (808.16 mg, 1.64 mmol) in DMF (5.0 mL) and H₂O (1.0 mL) were added K₃PO₄ (791.67 mg, 3.73 mmol) and Pd(dppf)Cl₂ (109.16 mg, 0.15 mmol). The resulting mixture was stirred for 2 h at 70° C. under an argon atmosphere. The mixture was purified by reverse phase column chromatography to afford (2S)-2-[(tert-butoxycarbonyl) amino]-3-[5-[(2M)-1-ethyl-3-(3-hydroxy-2,2-dimethylpropyl)-2-[2-[(1 S)-1-methoxyethyl]pyridin-3-yl]indol-5-yl]-1,3-oxazol-2-yl]propanoic acid (600 mg, 64.79% yield) as a light brown solid. LCMS (ESI): m/z [M+H] calc'd for C₃₄H₄₄N₄O₇ 621.33; found 621.3.

Step 8. To a stirred mixture of methyl (3S)-1,2-diazinane-3-carboxylate (627.10 mg, 4.350 mmol) and (2S)-2-[(tert-butoxycarbonyl) amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoic acid (900.0 mg, 1.45 mmol) in DCM (10.0 mL) were added HATU (661.54 mg, 1.74 mmol) and DIPEA (3747.71 mg, 29.00 mmol) at 0° C. The resulting mixture was stirred for 2 h. Desired product could be detected by LCMS. The resulting mixture was concentrated under reduced pressure and the crude material was purified by silica gel column chromatography to afford methyl (3S)-1-[(2S)-2-[(tert-butoxycarbonyl) amino]-3-[5-[(2M)-1-ethyl-3-(3-hydroxy-2,2-dimethylpropyl)-2-[2-[(1 S)-1-methoxyethyl]pyridin-3-yl]indol-5-yl]-1,3-oxazol-2-yl]propanoyl]-1,2-diazinane-3-carboxylate (900 mg, 83.11%) as a brown yellow solid. LCMS (ESI): m/z [M+H] calc'd for C₄₀H₅₄N₆O₈ 747.41; found 747.2.

Step 9. To a stirred mixture of methyl (3S)-1-[(2S)-2-[(tert-butoxycarbonyl) amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylate (2000.0 mg, 2.68 mmol) in THF (18. mL) and H₂O (6.0 mL) was added LiOH·H₂O (337.10 mg, 8.03 mmol) at 0° C. The resulting mixture was stirred for 2 h. Desired product could be detected by LCMS. The reaction was quenched with H₂O at 0° C. and adjusted to pH 6 with 1N HCl solution. The resulting mixture was extracted with EtOAc (3×50 mL). The combined organic layers were washed with brine (1×10 mL), dried over anhydrous Na₂SO₄. After filtration, the filtrate was concentrated under reduced pressure to afford (3S)-1-[(2S)-2-[(tert-butoxycarbonyl) amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylic acid (1300 mg, 66.2% yield) as a yellow solid. LCMS (ESI): m/z [M+H] calc'd for C₃₉H₅₂N₆O₈ 733.39; found 733.3.

Step 10. To a stirred mixture of (3S)-1-[(2S)-2-[(tert-butoxycarbonyl) amino]-3-[5-[(2M)-1-ethyl-3-(3-hydroxy-2,2-dimethylpropyl)-2-[2-[(1S)-1-methoxyethyl]pyridin-3-yl]indol-5-yl]-1,3-oxazol-2-yl]propanoyl]-1,2-diazinane-3-carboxylic acid (1.2 g, 1.64 mmol) and DIPEA (8.5 g, 65.50 mmol) in DCM (120.0 mL) were added HOBT (1.8 g, 13.10 mmol) and EDCI (7.8 g, 40.93 mmol) at 0° C.

The resulting mixture was stirred for 2 h. Desired product could be detected by LCMS. The mixture was concentrated under reduced pressure and purified by silica gel column chromatography to afford tert-butyl ((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl) carbamate (660 mg, 56.4% yield) as a brown yellow solid. LCMS (ESI): m/z [M+H] calc'd for C₃₉H₅₀N₆O₇ 715.38; found 715.3.

Step 11. A mixture of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)- pyridazinacycloundecaphane-4-yl) carbamate (20.0 mg, 0.028 mmol) and TFA (3.0 mL) in DCM (6.0 mL) at 0° C. was stirred for 2 h. Desired product could be detected by LCMS. The resulting mixture was concentrated under reduced pressure to afford (6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (5,2)-oxazola-1 (5,3)-indola-6 (1,3)-pyridazinacycloundecaphane-5,7-dione (160 mg, crude) as a yellow green solid. LCMS (ESI): m/z [M+H] calc'd for C₃₄H₄₂N₆O₅ 615.33; found 615.2.

Step 12. To a stirred mixture of (6³S,4S)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (5,2)-oxazola-1 (5,3)-indola-6 (1,3)-pyridazinacycloundecaphane-5,7-dione (180.0 mg, 0.293 mmol) and (2S)-2-[(tert-butoxycarbonyl) (methyl) amino]-3-methylbutanoic acid (135.45 mg, 0.59 mmol) in DMF (2.0 mL) were added HATU (133.60 mg, 0.35 mmol) and DIPEA (756.86 mg, 5.86 mmol) at 0° C. The resulting mixture was stirred for 2 h. Desired product could be detected by LCMS. The mixture was purified by reverse phase column chromatography to afford tert-butyl ((2S)-1-(((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl) amino)-3-methyl-1-oxobutan-2-yl) (methyl) carbamate (160 mg, 66% yield) as a brown yellow solid. LCMS (ESI): m/z [M+H] calc'd for C₄₅H₆₁N₇O₈ 828.47; found 828.4.

Step 13. To a stirred mixture of tert-butyl ((2S)-1-(((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl) amino)-3-methyl-1-oxobutan-2-yl) (methyl) carbamate (160.0 mg, 0.19 mmol) in DCM (2.0 mL) was added TFA (1.0 mL) at 0° C. The resulting mixture was stirred for 2 h. Desired product could be detected by LCMS. The resulting mixture was concentrated under reduced pressure to afford (2S)—N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl)-3-methyl-2-(methylamino) butanamide (130 mg, crude) as a yellow solid. LCMS (ESI): m/z [M+H] calc'd for C₄₀H₅₃N₇O₆ 728.41; found 728.5.

Step 14. To a stirred mixture of ((2S)—N-((6³S,4S)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl)-3-methyl-2-(methylamino) butanamide (130.0 mg, 0.18 mmol) and (3S)-1-[4-(dimethylamino)-4-methylpent-2-ynoyl]pyrrolidine-3-carboxylic acid (180.25 mg, 0.72 mmol) in DMF (2.0 mL) were added DIPEA (461.64 mg, 3.57 mmol) and HATU (135.81 mg, 0.36 mmol) at 0° C. The resulting mixture was stirred for 2 h. Desired product could be detected by LCMS. The mixture was purified by reverse phase column chromatography to afford (3S)-1-(4-(dimethylamino)-4-methylpent-2-ynoyl)-N-((2S)-1-(((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (5,2)-oxazola-1 (5,3)-indola-6 (1,3)-pyridazinacycloundecaphane-4-yl) amino)-3-methyl-1-oxobutan-2-yl)-N-methylpyrrolidine-3-carboxamide (55.3 mg, 31.3% yield) as a solid. ¹H NMR (400 MHZ, DMSO-d₆) δ 8.77 (dd, J=4.8, 1.7 Hz, 1H), 8.09-8.00 (m, 1H), 7.92 (s, 1H), 7.88-7.80 (m, 1H), 7.62 (d, J=8.7 Hz, 1H), 7.59-7.49 (m, 2H), 7.39-7.30 (m, 1H), 5.69 (p, J=8.8 Hz, 1H), 5.44 (d, J=12.1 Hz, 1H), 4.67 (d, J=10.7 Hz, 1H), 4.30-4.15 (m, 3H), 3.99 (dt, J=13.2, 6.4 Hz, 3H), 3.89-3.79 (m, 1H), 3.62 (ddd, J=30.5, 18.6, 11.4 Hz, 5H), 3.39 (dd, J=9.4, 3.8 Hz, 2H), 3.21-3.13 (m, 1H), 3.08 (d, J=15.0 Hz, 3H), 2.99-2.74 (m, 6H), 2.26-2.18 (m, 5H), 2.16 (s, 2H), 2.14-1.94 (m, 3H), 1.86-1.68 (m, 2H), 1.57 (q, J=9.2, 5.8 Hz, 1H), 1.44-1.27 (m, 9H), 0.94 (d, J=6.6 Hz, 4H), 0.89 (dd, J=6.5, 2.5 Hz, 2H), 0.80 (d, J=6.3 Hz, 2H), 0.77-0.69 (m, 4H), 0.58 (d, J=20.4 Hz, 3H). LCMS (ESI): m/z [M+H] calc'd for C₅₃H₇₁N₉O₈ 962.55; found 962.5.

### Example A675. Synthesis of (2R)-2-(((1-(4-(dimethylamino)-4-methylpent-2-ynoyl) azetidin-3-yl)oxy) methyl)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-3-methylbutanamide

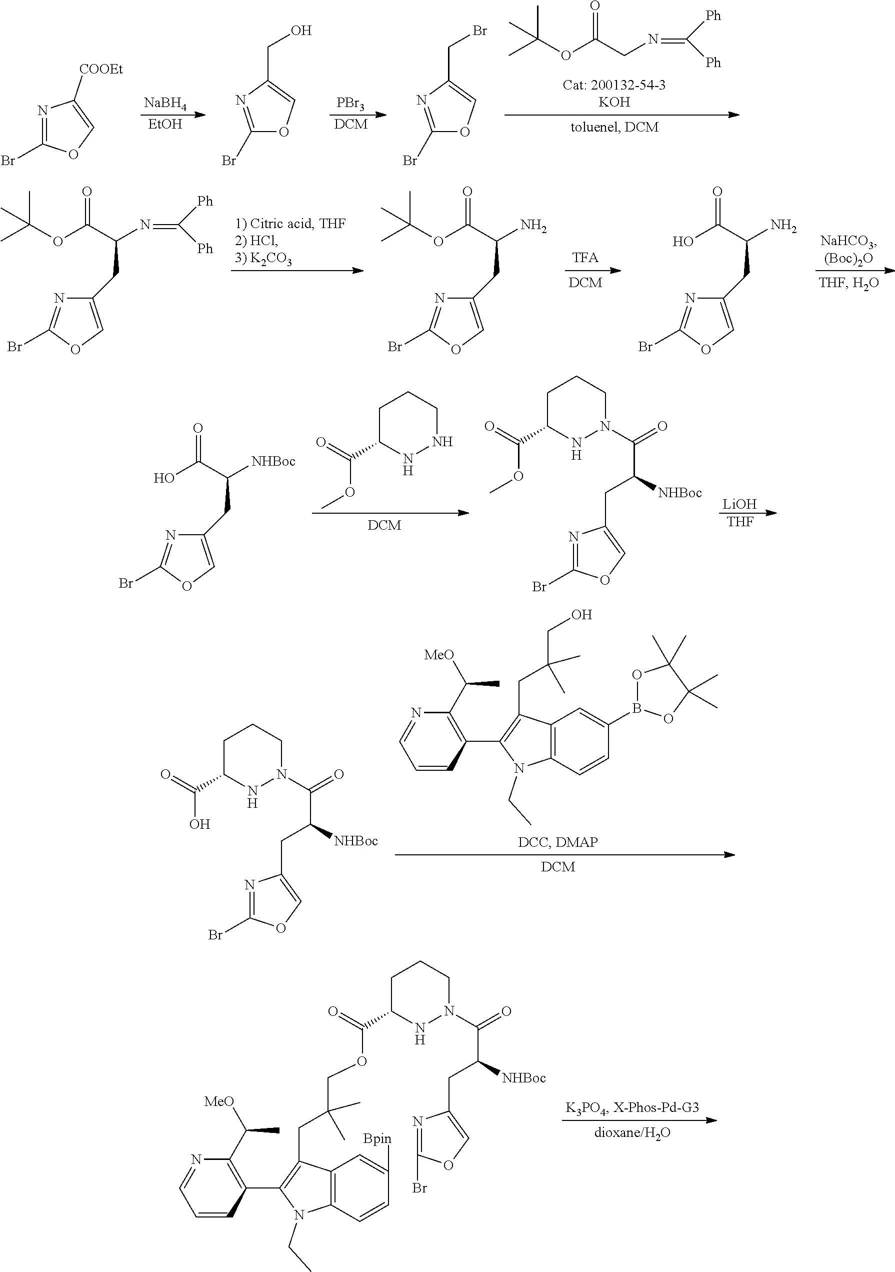

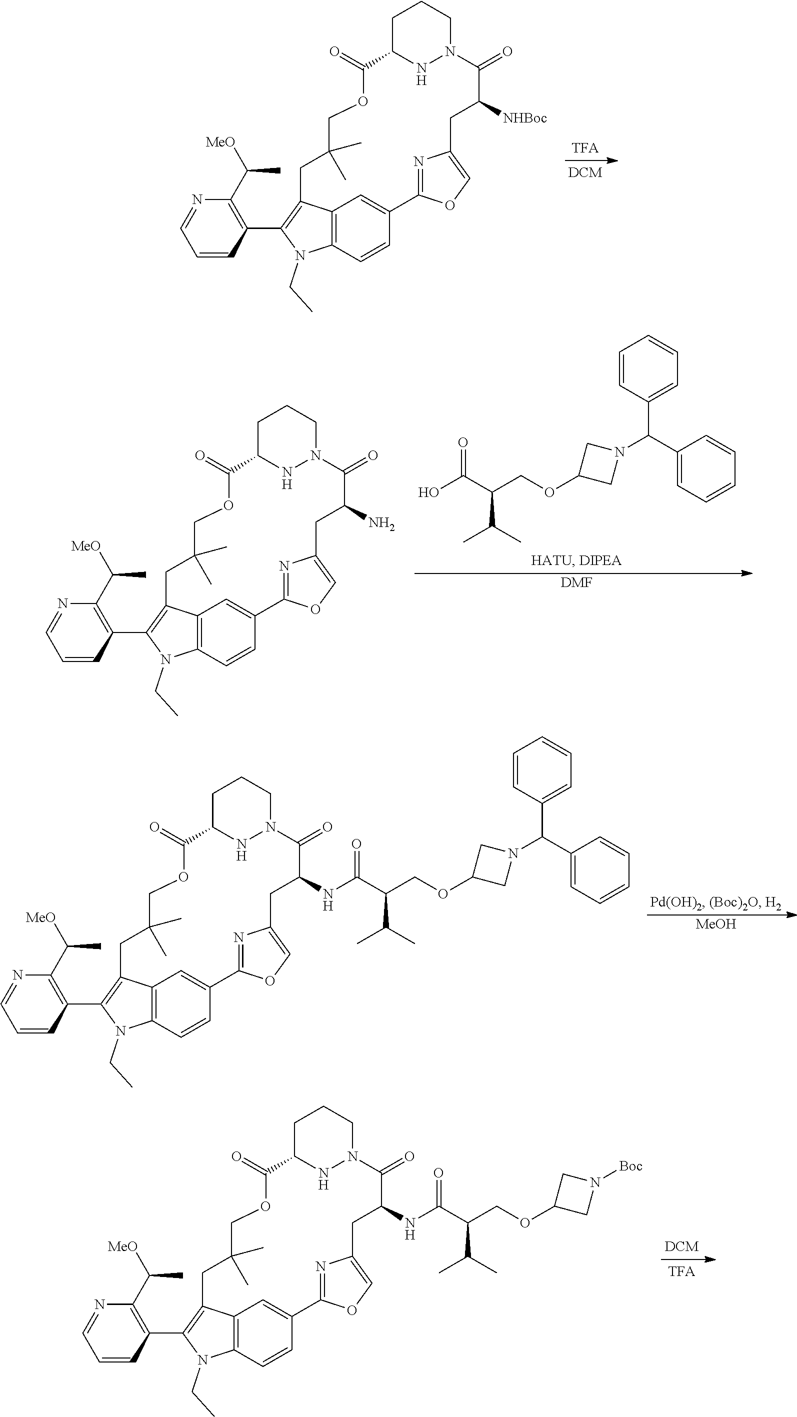

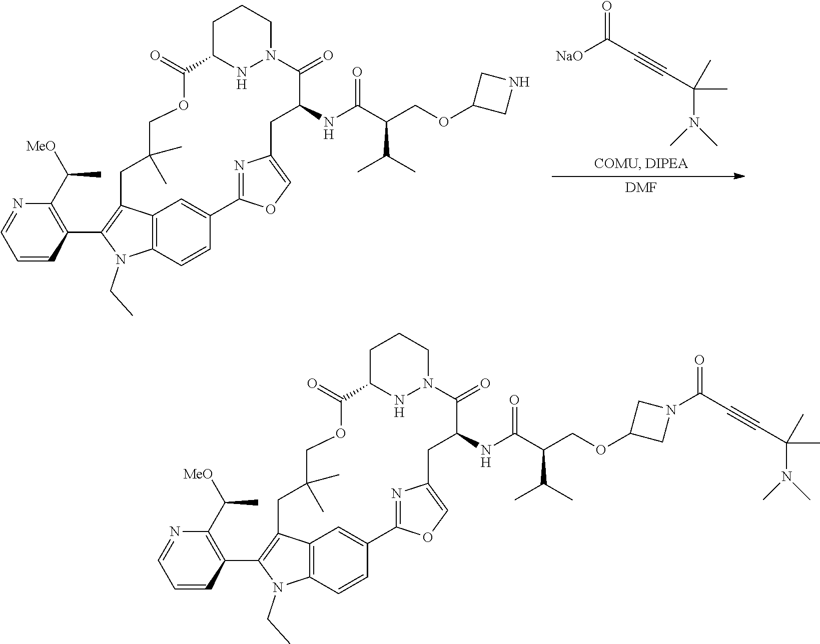

Step 1. A mixture of 2-bromo-4-(ethoxycarbonyl)-1,3-oxazol-5-ylium (6.83 g, 31.19 mmol), EtOH (100.0 mL) and NaBH₄ (4.72 g, 124.76 mmol) at 0° C. was stirred for 6 h at 0° C. under an air atmosphere. The reaction was quenched with H₂O at 0° C. The resulting mixture was extracted with EtOAc (3×100 mL). The combined organic layers were dried over anhydrous Na₂SO₄. After filtration, the filtrate was concentrated under reduced pressure to afford (2-bromo-1,3-oxazol-4-yl) methanol (4.122 g, 74.3% yield) as a solid. LCMS (ESI): m/z [M+H] calc'd for C₄H₄BrNO₂ 177.95; found 178.0.

Step 2. A mixture of (2-bromo-1,3-oxazol-4-yl) methanol (4.30 g, 24.16 mmol), DCM (50 mL) and phosphorus tribromide (9809.39 mg, 36.24 mmol) at 0° C. was stirred overnight at 0° C. under an air atmosphere. The reaction was quenched by the addition of NaHCO₃(aq.) at 0° C. The resulting mixture was extracted with EtOAc (3×100 mL). The combined organic layers were dried over anhydrous Na₂SO₄. After filtration, the filtrate was concentrated under reduced pressure to afford 2-bromo-4-(bromomethyl)-1,3-oxazole (3.28 g, 56.4% yield) as a liquid. LCMS (ESI): m/z [M+H] calc'd for C₄H₃Br₂NO 239.87; found 239.9.

Step 3. A mixture of 2-bromo-4-(bromomethyl)-1,3-oxazole (3280.0 mg, 13.62 mmol), KOH (9M, 10 mL), 30 mL mixture of toluene/DCM (7/3) and tert-butyl 2-[(diphenylmethylidene) amino]acetate (5228.74 mg, 17.70 mmol) at −16° C. was stirred overnight under an air atmosphere. The resulting mixture was extracted with EtOAc (3×50 mL). The combined organic layers were dried over anhydrous Na₂SO₄. After filtration, the filtrate was concentrated under reduced pressure to afford tert-butyl (2S)-3-(2-bromo-1,3-oxazol-4-yl)-2-[(diphenylmethylidene) amino]propanoate (8.33 g, 80.6% yield) as a solid. LCMS (ESI): m/z [M+H] calc'd for C₂₃H₂₃BrN₂O₃ 455.10; found 455.1.

Step 4. A mixture of tert-butyl (2S)-3-(2-bromo-1,3-oxazol-4-yl)-2-[(diphenylmethylidene) amino]propanoate (4100.0 mg, 9.0 mmol) and citric acid (1 N) (40.0 mL, 0.21 mmol), in THF (40 mL) at room temperature was stirred overnight under an air atmosphere. The reaction was quenched by the addition of HCl (aq.) (100 mL) at 0° C. The aqueous layer was extracted with EtOAc (3×100 mL). K₂CO₃ (aq.) (200 mL) was added to the resulting mixture and extracted with EtOAc (3×100 mL). The organic layer was concentrated under reduced pressure to afford tert-butyl (2S)-2-amino-3-(2-bromo-1,3-oxazol-4-yl) propanoate (1730 mg, 66% yield) as a dark yellow solid. LCMS (ESI): m/z [M+H] calc'd for C₁₀H₁₅BrN₂O₃ 291.03; found 291.0.

Step 5. A mixture of tert-butyl (2S)-2-amino-3-(2-bromo-1,3-oxazol-4-yl) propanoate (1780.0 mg, 6.11 mmol), TFA (10.0 mL) and DCM (10.0 mL) at 0° C. was stirred for overnight under an air atmosphere. The resulting mixture was concentrated under reduced pressure to afford (2S)-2-amino-3-(2-bromo-1,3-oxazol-4-yl) propanoic acid (1250 mg, 87% yield) as a dark yellow solid. LCMS (ESI): m/z [M+H] calc'd for C₆H₇BrN₂O₃ 234.97; found 234.9.

Step 6. A mixture of di-tert-butyl dicarbonate (4178.58 mg, 19.15 mmol), THF (10 mL), H₂O (10 mL), (2S)-2-amino-3-(2-bromo-1,3-oxazol-4-yl) propanoic acid (1500.0 mg, 6.38 mmol) and NaHCO₃ (3216.74 mg, 38.29 mmol) at room temperature was stirred overnight under an air atmosphere. The reaction was quenched with H₂O at room temperature. The resulting mixture was concentrated under reduced pressure. The resulting mixture was extracted with EtOAc (3×100 mL). The aqueous layer was acidified to pH 6 with 1 M HCl (aq.). The resulting mixture was extracted with EtOAc (3×100 mL). The combined organic layers were dried over anhydrous Na₂SO₄. After filtration, the filtrate was concentrated under reduced pressure to afford (2S)-3-(2-bromo-1,3-oxazol-4-yl)-2-[(tert-butoxycarbonyl) amino]propanoic acid (920 mg, 43.0% yield) as an oil. LCMS (ESI): m/z [M+H] calc'd for C₁₁H₁₅BrN₂O₅ 335.02; found 335.0.

Step 7. A mixture of 3-(2-bromo-1,3-oxazol-4-yl)-2-[(tert-butoxycarbonyl) amino]propanoic acid (850.0 mg, 2.54 mmol), methyl 1,2-diazinane-3-carboxylate (1.88 g, 13.04 mmol), DIPEA (1966.68 mg, 15.22 mmol), DCM (30.0 mL) and HATU (1446.48 mg, 3.80 mmol) at 0° C. was stirred for 3 h under an air atmosphere. The resulting mixture was extracted with DCM (3×50 mL). The combined organic layers were dried over anhydrous Na₂SO₄. After filtration, the filtrate was concentrated under reduced pressure. The resulting mixture was purified by reverse flash chromatography to afford methyl 1-[3-(2-bromo-1,3-oxazol-4-yl)-2-[(tert-butoxycarbonyl) amino]propanoyl]-1,2-diazinane-3-carboxylate (610 mg, 52.1% yield) as a solid. LCMS (ESI): m/z [M+H] calc'd for C₁₇H₂₅BrN₄O₆ 461.10; found 461.0.

Step 8. A mixture of methyl 1-[3-(2-bromo-1,3-oxazol-4-yl)-2-[(tert-butoxycarbonyl) amino]propanoyl]-1,2-diazinane-3-carboxylate (570.0 mg, 1.24 mmol), LiOH (2.0 mL, 1 M aq.) and THF (2.0 mL) at 0° C. was stirred for 3 h under an air atmosphere. The resulting mixture was concentrated under reduced pressure. The resulting mixture was extracted with EtOAc (3×50 mL). The combined aqueous layers were acidified to pH 5 with 1N HCl (aq.). The aqueous phase was extracted with EtOAc (3×50 mL). The combined organic layers were dried over anhydrous Na₂SO₄. After filtration, the filtrate was concentrated under reduced pressure to afford 1-[3-(2-bromo-1,3-oxazol-4-yl)-2-[(tert-butoxycarbonyl) amino]propanoyl]-1,2-diazinane-3-carboxylic acid (500 mg, 90.5% yield) as a solid. LCMS (ESI): m/z [M+H] calc'd for C₁₆H₂₃BrN₄O₆ 447.09; found 446.8.

Step 9. A mixture of 1-[3-(2-bromo-1,3-oxazol-4-yl)-2-[(tert-butoxycarbonyl) amino]propanoyl]-1,2-diazinane-3-carboxylic acid (450.0 mg, 1.01 mmol), 3-[(2M)-1-ethyl-2-[2-[(1S)-1-methoxyethyl]pyridin-3-yl]-5-(4,4,5,5-tetramethyl-1,3,2-dioxaborolan-2-yl) indol-3-yl]-2,2-dimethylpropan-1-ol (743.19 mg, 1.51 mmol), DMAP (24.58 mg, 0.20 mmol), DCM (15.0 mL) and DCC (311.37 mg, 1.51 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((S)-3-(2-bromooxazol-4-yl)-2-((tert-butoxycarbonyl) amino) propanoyl) hexahydropyridazine-3-carboxylate (330 mg, 35.6% yield) as a white solid. LCMS (ESI): m/z [M+H] calc'd for C₄₅H₆₂BBrN₆O₉ 921.39; found 921.4.

Step 10. A mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((S)-3-(2-bromooxazol-4-yl)-2-((tert-butoxycarbonyl) amino) propanoyl) hexahydropyridazine-3-carboxylate (290.0 mg, 0.33 mmol), K₃PO₄ (206.64 mg, 0.97 mmol), X-Phos (30.94 mg, 0.07 mmol), XPhos Pd G3 (54.93 mg, 0.07 mmol), dioxane (5. mL) and H₂O (1.0 mL) at 70° C. was stirred for 4 h under an argon atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl) carbamate (130 mg, 56.0% yield) as a solid. LCMS (ESI): m/z [M+H] calc'd for C₃₉H₅₀N₆O₇ 715.38; found 715.3.

Step 11. A mixture of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)- pyridazinacycloundecaphane-4-yl) carbamate (120.0 mg), DCM (2.0 mL) and TFA (0.2 mL) at room temperature was stirred for 6 h under an air atmosphere. The resulting mixture was concentrated under reduced pressure. to afford (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (2,4)-oxazola-1 (5,3)-indola-6 (1,3)-pyridazinacycloundecaphane-5,7-dione (90 mg, 87.2% yield) as a solid. LCMS (ESI): m/z [M+H] calc'd for C₃₄H₄₂N₆O₅ 615.33; found 615.3.

Step 12. A mixture of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (2,4)-oxazola-1 (5,3)-indola-6 (1,3)-pyridazinacycloundecaphane-5,7-dione (200.0 mg, 0.33 mmol), (2R)-2-([[1-(diphenylmethyl) azetidin-3-yl]oxy]methyl)-3-methylbutanoic acid (172.49 mg, 0.49 mmol), DIPEA (420.48 mg, 3.25 mmol), DMF (3.0 mL) and HATU (148.44 mg, 0.39 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The residue was purified by Prep-TLC to afford (2R)-2-(((1-benzhydrylazetidin-3-yl)oxy) methyl)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)- pyridazinacycloundecaphane-4-yl)-3-methylbutanamide (154 mg, 77.0% yield) as a solid. LCMS (ESI): m/z [M+H] calc'd for C₅₆H₆₇N₇O₇ 950.52; found 950.6.

Step 13. A mixture of (2R)-2-(((1-benzhydrylazetidin-3-yl)oxy) methyl)-N-((6³S,4S,Z)-11-ethyl-1²-(2-((S)-1-methoxyethyl) pyridin-3-yl)-10, 10-dimethyl-5,7-dioxo-6¹,6²,6³,6⁴,6⁵,6⁶-hexahydro-11H-8-oxa-2 (2,4)-oxazola-1 (5,3)-indola-6 (1,3)-pyridazinacycloundecaphane-4-yl)-3-methylbutanamide (240.0 mg, 0.25 mmol), (Boc)₂O (165.37 mg, 0.76 mmol), MeOH (5.0 mL) and Pd(OH)₂ (72.0 mg, 0.51 mmol) at room temperature was stirred overnight under an H₂ atmosphere. The resulting mixture was filtered, the filter cake was washed with MeOH (3×5 mL). The filtrate was concentrated under reduced pressure. The residue was purified by Prep-TLC to afford tert-butyl 3-((2R)-2-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl) carbamoyl)-3-methylbutoxy) azetidine-1-carboxylate (150 mg, 67.2% yield) as a solid. LCMS (ESI): m/z [M+H] calc'd for C₄₈H₆₅N₇O₉ 884.49; found 884.2.

Step 14. A mixture of tert-butyl 3-((2R)-2-(((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl) carbamoyl)-3-methylbutoxy) azetidine-1-carboxylate (150.0 mg), DCM (2.0 mL) and TFA (0.40 mL) at 0° C. was stirred for 3 h under an air atmosphere. The resulting mixture was concentrated under reduced pressure to afford (2R)-2-((azetidin-3-yloxy) methyl)-N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-3-methylbutanamide (120 mg, 90.2% yield) as a solid. LCMS (ESI): m/z [M+H] calc'd for C₄₃H₅₇N₇O₇ 784.44; found 784.2.

Step 15. A mixture of (2R)-2-((azetidin-3-yloxy) methyl)-N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-3-methylbutanamide (130.0 mg, 0.17 mmol), sodium 4-(dimethylamino)-4-methylpent-2-ynoate (44.07 mg, 0.25 mmol), DMF (3.0 mL), DIPEA (64.29 mg, 0.50 mmol) and COMU (106.46 mg, 0.25 mmol) at 0° C. was stirred for 3 h under an air atmosphere. The resulting mixture was extracted with EtOAc (3×20 mL). The combined organic layers were dried over anhydrous Na₂SO₄. After filtration, the filtrate was concentrated under reduced pressure. The crude product was purified by reverse phase chromatography to afford (2R)-2-(((1-(4-(dimethylamino)-4-methylpent-2-ynoyl) azetidin-3-yl)oxy) methyl)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-3-methylbutanamide (25 mg, 16.4% yield) as a solid. ¹H NMR (400 MHZ, DMSO-d₆) δ 8.77 (dd, J=4.8, 1.8 Hz, 1H), 8.55 (s, 1H), 8.14 (dd, J=8.3, 2.9 Hz, 1H), 7.82 (d, J=7.6 Hz, 1H), 7.76-7.65 (m, 3H), 7.54 (dd, J=7.7, 4.7 Hz, 1H), 7.54-7.02 (m, 1H), 5.72 (td, J=7.4, 3.4 Hz, 1H), 4.97 (d, J=11.9 Hz, 1H), 4.46-4.24 (m, 6H), 4.18-4.04 (m, 2H), 3.94 (dd, J=32.9, 7.8 Hz, 1H), 3.77-3.63 (m, 2H), 3.57 (s, 1H), 3.49 (s, 2H), 3.21 (s, 3H), 2.90 (d, J=14.6 Hz, 1H), 2.87-2.79 (m, 1H), 2.72 (td, J=15.5, 14.6, 3.1 Hz, 2H), 2.46 (s, 1H), 2.43-2.26 (m, 6H), 2.11-1.99 (m, 1H), 1.82-1.66 (m, 2H), 1.56-1.37 (m, 11H), 0.89 (dt, J=12.3, 7.7 Hz, 12H), 0.35 (s, 3H). LCMS (ESI): m/z [M+H] calc'd for C₅₁H₆₈N₈O₈ 921.52; found 921.5.

### Example A607. The synthesis of (2R)-2-(((1-(4-(dimethylamino)-4-methylpent-2-ynoyl) azetidin-3-yl)oxy) methyl)-N-((23S,6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-piperidinacycloundecaphane-4-yl)-3-methylbutanamide

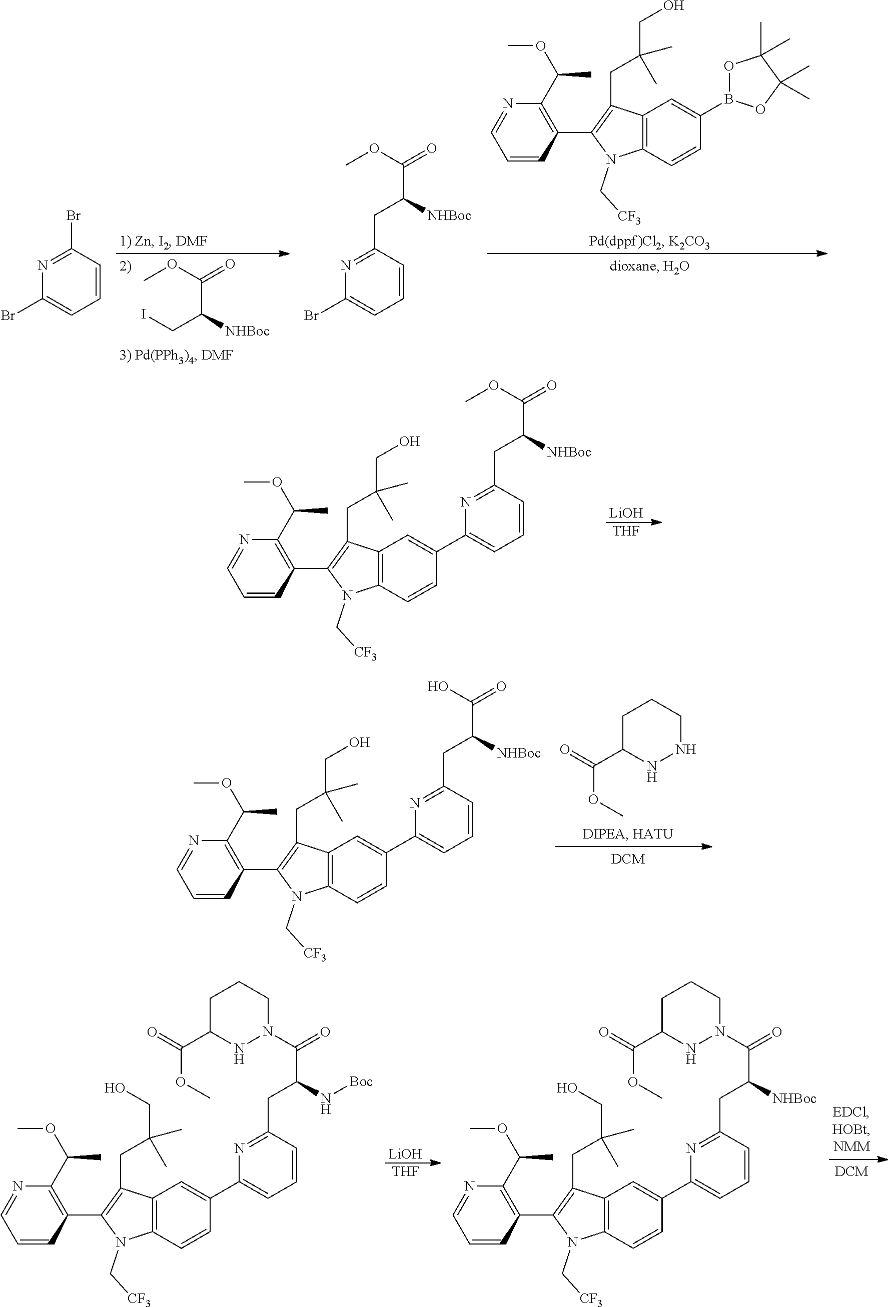

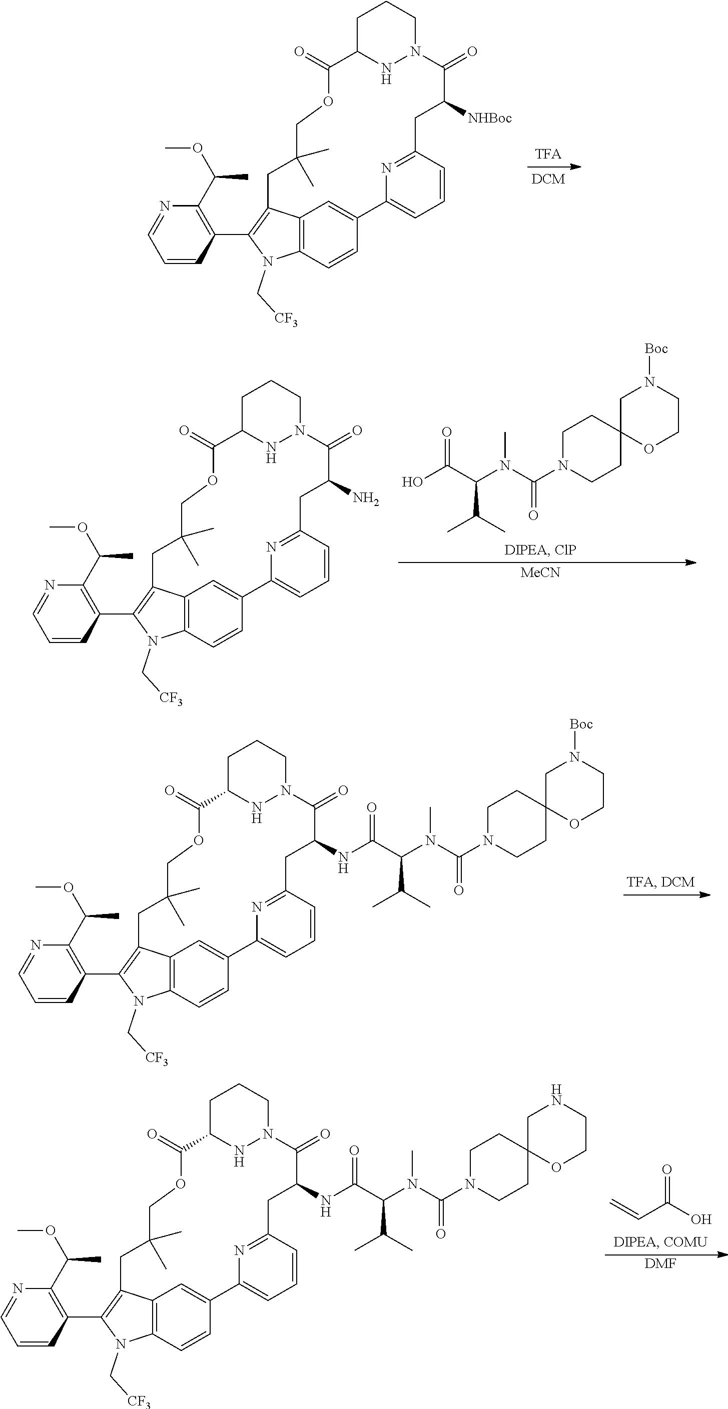

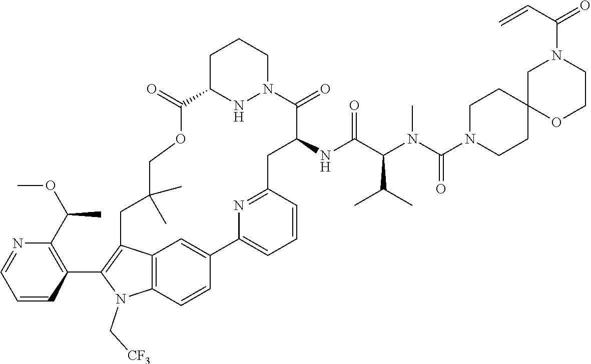

Step 1. A mixture of Zn (44.18 g, 675.41 mmol) and 12 (8.58 g, 33.77 mmol) in DMF (120 mL) was stirred for 30 min at 50° C. under an argon atmosphere, followed by the addition of methyl (2R)-2-[(tert-butoxycarbonyl) amino]-3-iodopropanoate (72.24 g, 219.51 mmol) in DMF (200 mL). The reaction mixture was stirred at 50° C. for 2 h under an argon atmosphere. Then a mixture of 2,6-dibromo-pyridine (40 g, 168.85 mmol) and Pd(PPh₃)₄ (39.02 g, 33.77 mmol) in DMF (200 mL) was added. The resulting mixture was stirred at 75° C. for 2 h, then cooled down to room temperature and extracted with EtOAc (1 L×3). The combined organic layers were washed with H₂O (1 L×3), dried over anhydrous sodium sulfate, filtered and concentrated under reduced pressure. The residue was purified by silica gel column chromatography to afford methyl (2S)-3-(6-bromopyridin-2-yl)-2-[(tert-butoxycarbonyl) amino]propanoate (41 g, 67% yield) as oil. LCMS (ESI): m/z [M+H] calc'd for C₁₄H₁₉BrN₂O₄ 358.1; found 359.1.

Step 2. To a solution of 3-[(2M)-2-[2-[(1S)-1-methoxyethyl]pyridin-3-yl]-5-(4,4,5,5-tetramethyl-1,3,2-dioxaborolan-2-yl)-1-(2,2,2-trifluoroethyl) indol-3-yl]-2,2-dimethylpropan-1-ol (45.0 g, 82.35 mmol) in dioxane (400 mL) and H₂O (80 mL), were added potassium carbonate (28.45 g, 205.88 mmol), methyl (2S)-3-(6-bromopyridin-2-yl)-2-[(tert-butoxycarbonyl) amino]propanoate (35.5 g, 98.8 mmol), Pd(dtbpf)Cl₂ (5.37 g, 8.24 mmol) at room temperature. The reaction mixture was stirred at 70° C. for 2 h under a nitrogen atmosphere. The resulting mixture was extracted with EtOAc (500 mL×3). The combined organic layers were washed with H₂O (300 mL×3), dried over anhydrous sodium sulfate, filtered and concentrated under reduced pressure. The residue was purified by silica gel chromatography to afford methyl(S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoate (48 g, 83% yield) as solid. LCMS (ESI): m/z [M+H] calc'd for C₃₇H₄₅F₃N₄O₆ 698.3; found 699.4.

Step 3. A solution of methyl(S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoate (52 g, 74.42 mmol) in THF (520 mL), was added LiOH (74.41 mL, 223.23 mmol) at 0° C. The reaction mixture was stirred at room temperature for 3 h. The resulting mixture was acidified to pH 5 with HCl (aq.) and extracted with EtOAc (1 L×3). The combined organic layers were washed with H₂O (1 L×3), dried over anhydrous sodium sulfate, filtered and concentrated under reduced pressure to afford(S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoic acid (50 g, 98% yield) as solid. LCMS (ESI): m/z [M+H] calc'd for C₃₆H₄₃F₃N₄O₆ 684.3; found 685.1.

Step 4. To a solution of(S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoic acid (55 g, 80.32 mmol) in DCM (600 mL), were added DIPEA (415.23 g, 3212.82 mmol), and HATU (45.81 g, 120.48 mmol) at 0° C. The reaction mixture was stirred at room temperature for 12 h and then quenched with H₂O. The resulting mixture was extracted with EtOAc (1 L×3). The combined organic layers were washed with H₂O (1 L), dried over anhydrous sodium sulfate, filtered and concentrated under reduced pressure. The residue was purified by silica gel column chromatography to afford methyl 1-((S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoyl) hexahydropyridazine-3-carboxylate (63 g, 96% yield) as solid. LCMS (ESI): m/z [M+H] calc'd for C₄₂H₅₃F₃N₆O₇ 810.4; found 811.3.

Step 5. A solution of methyl 1-((S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoyl) hexahydropyridazine-3-carboxylate (50 g, 61.66 mmol) in THF (500 mL) and 3M LiOH (61.66 mL, 184.980 mmol) at 0° C. was stirred at room temperature for 3 h, then acidified to pH 5 with HCl (aq.). The resulting mixture was extracted with EtOAc (800 mL×3). The combined organic layers were washed with H₂O (800 mL), dried over anhydrous sodium sulfate, filtered and concentrated under reduced pressure to afford 1-((S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoyl) hexahydropyridazine-3-carboxylic acid (48 g, 97% yield) as solid. LCMS (ESI): m/z [M+H] calc'd for C₄₁H₅₁F₃N₆O₇ 796.3; found 797.1.

Step 6. To a solution of 1-((S)-2-((tert-butoxycarbonyl) amino)-3-(6-(3-(3-hydroxy-2,2-dimethylpropyl)-2-(2-((S)-1-methoxyethyl) pyridin-3-yl)-1-(2,2,2-trifluoroethyl)-1H-indol-5-yl) pyridin-2-yl) propanoyl) hexahydropyridazine-3-carboxylic acid (50 g, 62.74 mmol) in DCM (10 L) at 0° C., were added DIPEA (243.28 g, 1882.32 mmol), EDCI (360.84 g, 1882.32 mmol) and HOBT (84.78 g, 627.44 mmol). The reaction mixture was stirred at room temperature for 3 h, quenched with H₂O and concentrated under reduced pressure. The residue was extracted with EtOAc (2 L×3). The combined organic layers were washed with H₂O (2 L×3), dried over anhydrous sodium sulfate, filtered and concentrated under reduced pressure. The residue was purified by silica gel column chromatography to afford tert-butyl ((4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) carbamate (43.6 g, 89% yield) as solid. LCMS (ESI): m/z [M+H] calc'd for C₄₁H₄₉F₃N₆O₆ 778.3; found 779.3.

Step 7. To a solution of tert-butyl ((4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) carbamate (300 mg) in DCM (10 mL), was added TFA (3 mL) at 0° C. The reaction mixture was stirred at room temperature for 1 h. The resulting mixture was diluted with toluene (10 mL) and concentrated under reduced pressure three times to afford (4S)-4-amino-12-(2-((S)-1-methoxyethyl) pyridin-3-yl)-10, 10-dimethyl-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-5,7-dione (280 mg, crude) as oil. LCMS (ESI): m/z [M+H] calc'd for C₃₆H₄₁F₃N₆O₄ 679.2; found 678.3.

Step 8. To a solution of (4S)-4-amino-12-(2-((S)-1-methoxyethyl) pyridin-3-yl)-10,10-dimethyl-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-5,7-dione (140 mg, 0.21 mmol) in MeCN (2 mL), were added DIPEA (266.58 mg, 2.06 mmol), N-(4-(tert-butoxycarbonyl)-1-oxa-4,9-diazaspiro[5.5]undecane-9-carbonyl)-N-methyl-L-valine (127.94 mg, 0.31 mmol) and CIP (114.68 mg, 0.41 mmol) at 0° C. The reaction mixture was stirred at room temperature for 1 h. The resulting mixture was concentrated under reduced pressure. The residue was extracted with EtOAc (10 mL×3). The combined organic layers were washed with H₂O (10 mL×3), dried over anhydrous sodium sulfate, filtered and concentrated under reduced pressure. The residue was purified by silica gel chromatography to afford tert-butyl 9-(((2S)-1-(((6³S,4S)-1²-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) amino)-3-methyl-1-oxobutan-2-yl) (methyl) carbamoyl)-1-oxa-4,9-diazaspiro[5.5]undecane-4-carboxylate (170 mg, 76% yield) as solid. LCMS (ESI): m/z [M+H] calc'd for C₅₆H₇₄F₃N₉O₉ 1073.5; found 1074.6.

Step 9. To a solution of tert-butyl 9-(((2S)-1-(((6³S,4S)-1²-(2-((S)-1-methoxyethyl) pyridin-3-yl)-10, 10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) amino)-3-methyl-1-oxobutan-2-yl) (methyl) carbamoyl)-1-oxa-4,9-diazaspiro[5.5]undecane-4-carboxylate (160 mg, 0.15 mmol) in DCM (5 mL) at 0° C., was dropwise added TFA (1.5 mL). The reaction mixture was stirred at 0° C. for 1 h. The resulting mixture was diluted with toluene (10 mL) and concentrated under reduced pressure three times to afford N-((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) amino)-3-methyl-1-oxobutan-2-yl)-N-methyl-1-oxa-4,9-diazaspiro[5.5]undecane-9-carboxamide (150 mg, crude) as oil. LCMS (ESI): m/z [M+H] calc'd for C₅₁H₆₅F₃N₉O₇ 973.5; found 974.4.

Step 10. To a solution of N-((2S)-1-(((6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) amino)-3-methyl-1-oxobutan-2-yl)-N-methyl-1-oxa-4,9-diazaspiro[5.5]undecane-9-carboxamide (150 mg, 0.15 mmol) in DMF (3 mL), were added DIPEA (199.01 mg, 1.54 mmol), acrylic acid (16.64 mg, 0.23 mmol) and COMU (98.39 mg, 0.23 mmol) at 0° C. The reaction mixture was stirred at room temperature for 1 h and concentrated under reduced pressure. The residue was extracted with EtOAc (10 mL×3). The combined organic layers were washed with H₂O (10 mL×3), dried over anhydrous sodium sulfate, filtered and concentrated under reduced pressure. The residue was purified by silica gel chromatography and reverse phase chromatography to afford 4-acryloyl-N-((2S)-1-(((6³S,4S)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1 (5,3)-indola-6 (1,3)-pyridazina-2 (2,6)-pyridinacycloundecaphane-4-yl) amino)-3-methyl-1-oxobutan-2-yl)-N-methyl-1-oxa-4,9-diazaspiro[5.5]undecane-9-carboxamide (53 mg, 33% yield) as solid. ¹H NMR (400 MHZ, DMSO-d₆) δ 8.79 (dd, J=4.8, 1.9 Hz, 2H), 8.09 (d, J=31.4 Hz, 1H), 7.96 (d, J=8.9 Hz, 1H), 7.90-7.72 (m, 3H), 7.64 (t, J=7.8 Hz, 1H), 7.56 (dd, J=7.8, 4.7 Hz, 1H), 7.03 (s, 1H), 6.86 (dd, J=16.6, 10.4 Hz, 1H), 6.20 (d, J=14.0 Hz, 1H), 5.73 (d, J=12.2 Hz, 2H), 5.47 (s, 1H), 5.35 (d, J=11.8 Hz, 1H), 4.68 (s, 1H), 4.28 (d, J=12.5 Hz, 1H), 4.13 (d, J=6.4 Hz, 1H), 3.92 (s, 1H), 3.84-3.64 (m, 6H), 3.59 (d, J=14.0 Hz, 3H), 3.50 (s, 3H), 3.10 (s, 5H), 3.02 (d, J=13.3 Hz, 2H), 2.85 (d, J=12.1 Hz, 3H), 2.08-1.89 (m, 2H), 1.81 (s, 1H), 1.75-1.51 (m, 5H), 1.39 (d, J=6.1 Hz, 4H), 1.24 (s, OH), 0.91-0.66 (m, 10H), 0.53 (s, 3H). LCMS (ESI): m/z [M+H] calc'd for C₅₄H₆₈F₃N₉O₈ 1027.5; found 1028.1.

### Example A590. The synthesis of (2R)-2-(((1-(4-(dimethylamino)-4-methylpent-2-ynoyl) azetidin-3-yl)oxy) methyl)-N-((6³S,4S,Z)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl)-3-methylbutanamide

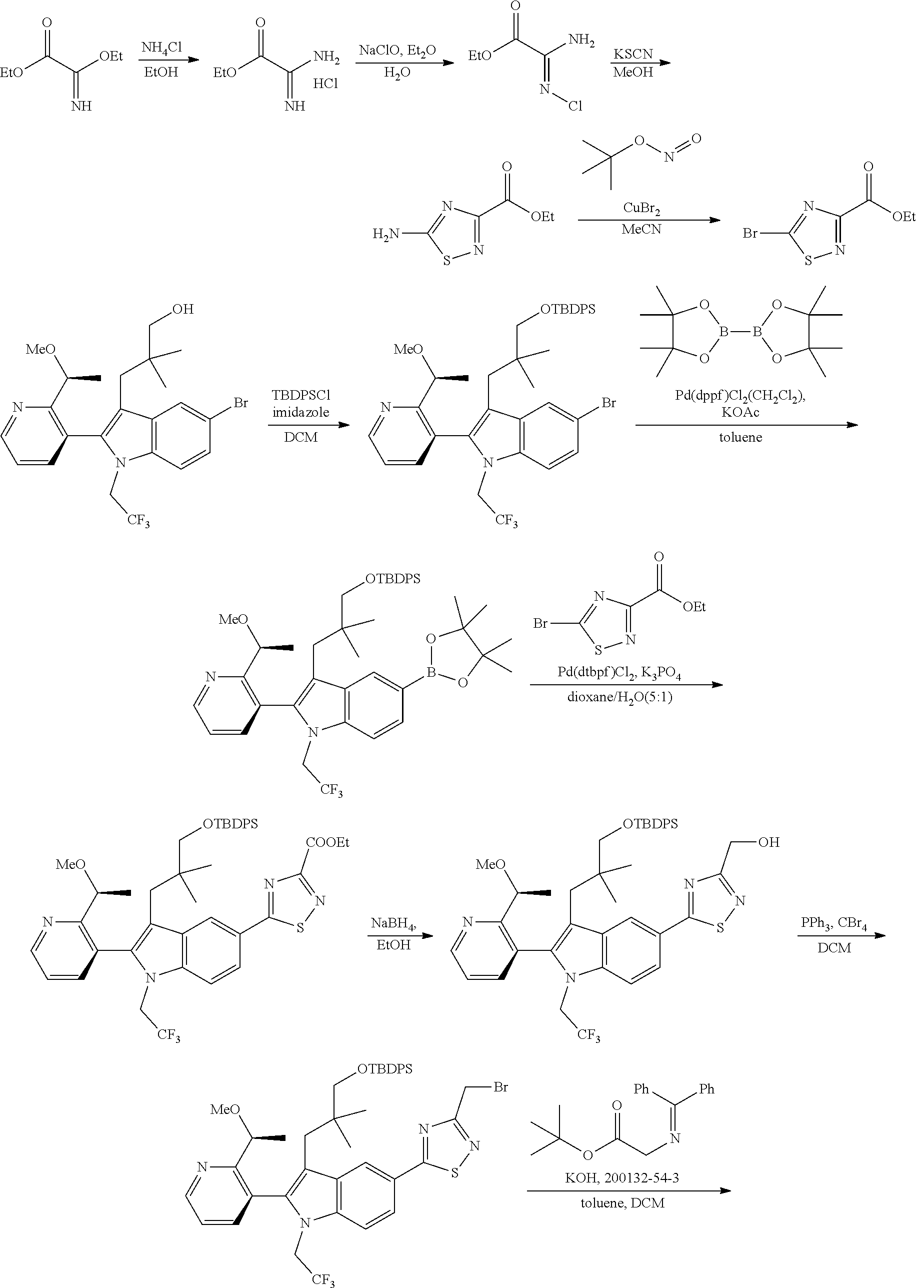

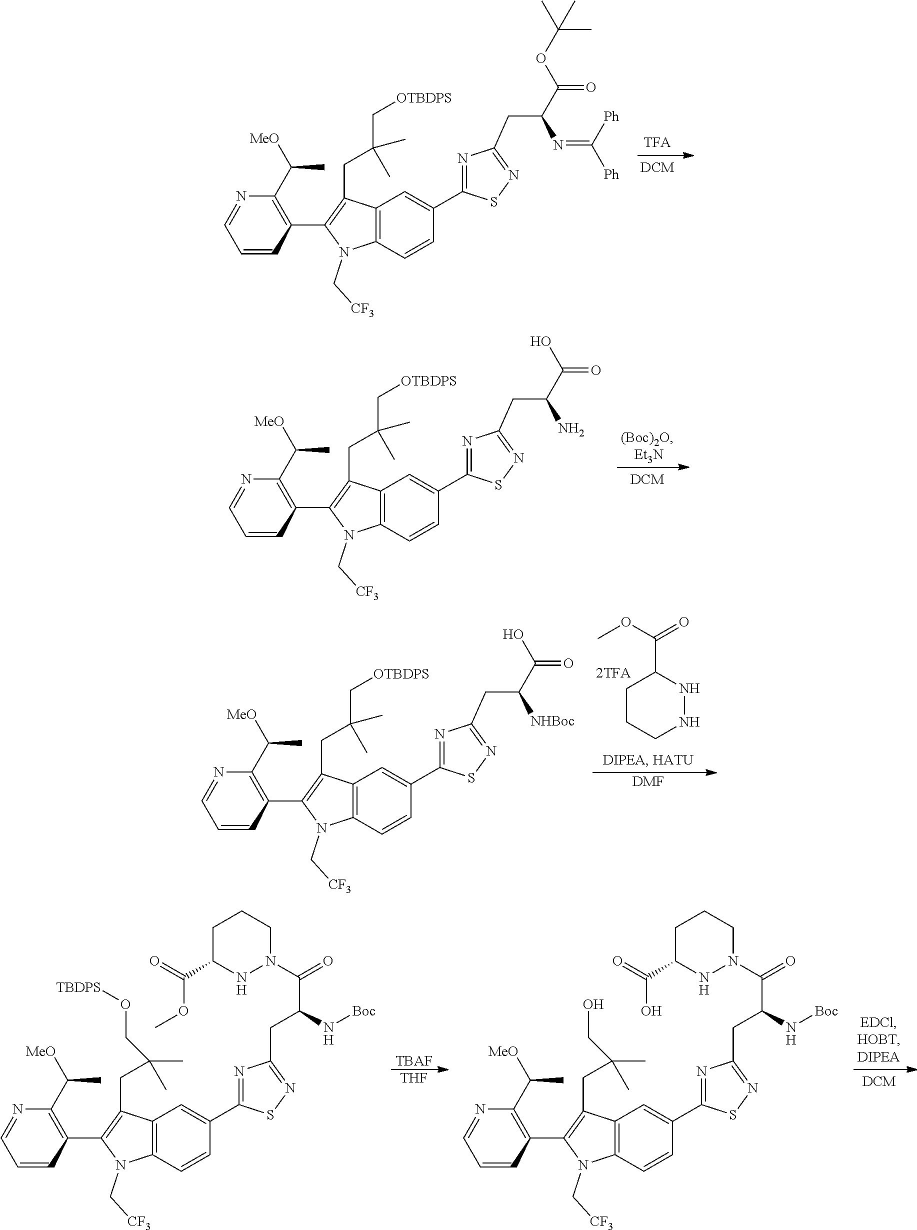

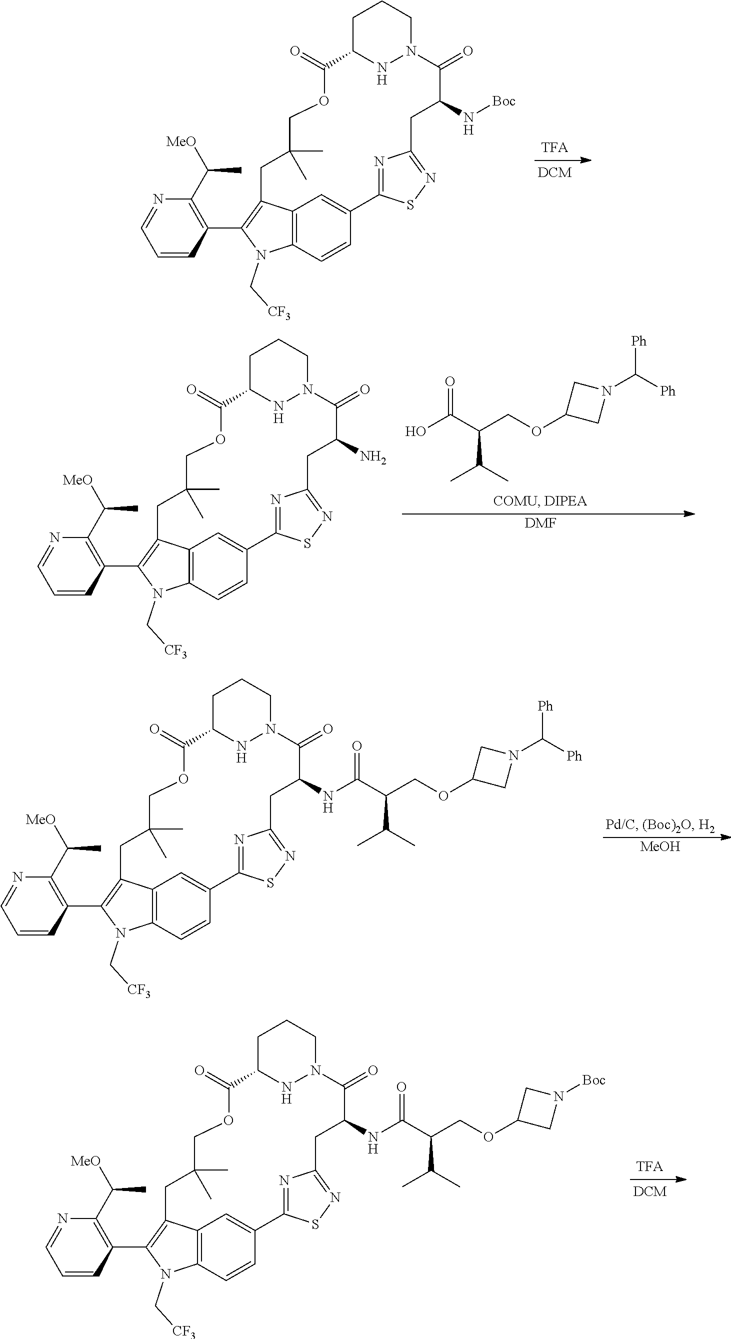

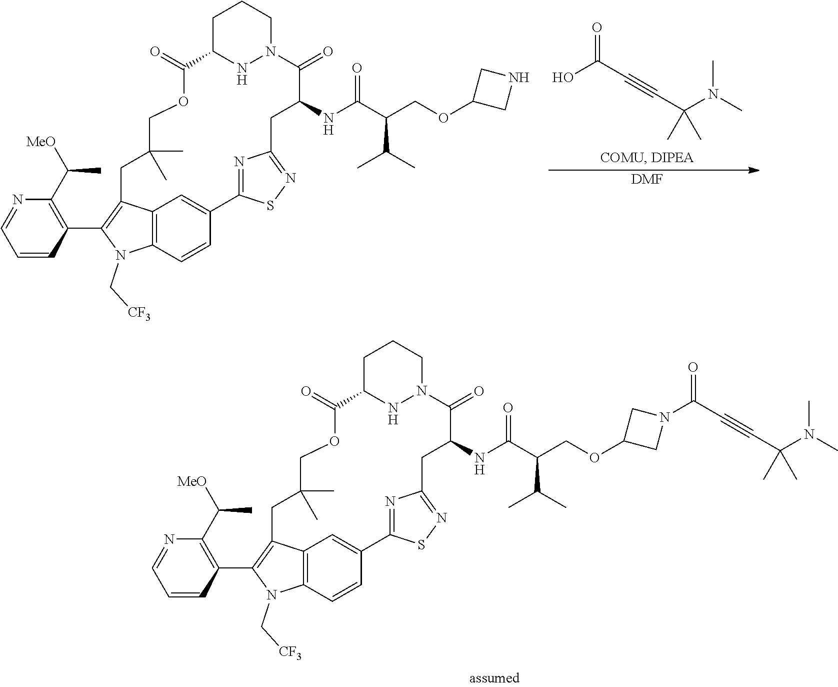

Step 1. To a mixture of ethyl 2-ethoxy-2-iminoacetate (25.0 g, 172.23 mmol) and EtOH (250.0 mL) at 0° C. was added ammonium chloride (9.21 g, 172.23 mmol) in portions then stirred for 4 h at room temperature under an argon atmosphere. The resulting mixture was concentrated under reduced pressure and washed with Et₂O (3×200 mL). The organic layers were combined and concentrated under reduced pressure. This resulted in ethyl 2-amino-2-iminoacetate hydrochloride (20 g, crude) as a light yellow solid. LCMS (ESI): m/z [M+H] calc'd for C₄H₃N₂O₂ 117.07; found 116.9.

Step 2. To a mixture of ethyl 2-amino-2-iminoacetate hydrochloride (13.30 g, 87.17 mmol), H₂O (50.0 mL) and Et₂O (100.0 mL) at 0° C. was added sodium hypochlorite pentahydrate (7.79 g, 104.60 mmol) dropwise. The resulting mixture was stirred for 3 h under an argon atmosphere. The mixture was extracted with Et₂O (3×200 mL). The resulting solution was washed with brine (3×100 mL). The organic phase was dried over anhydrous Na₂SO₄ and concentrated under reduced pressure to afford ethyl (Z)-2-amino-2-(chloroimino) acetate (7 g, crude) as a light yellow solid. LCMS (ESI): m/z [M+H] calc'd for C₄H₇ClN₂O₂ 151.03; found 150.8.

Step 3. To a solution of ethyl (Z)-2-amino-2-(chloroimino) acetate (8.40 g, 55.792 mmol) and MeOH (130.0 mL) at 0° C. was added potassium thiocyanate (5.42 g, 55.79 mmol) in portions. The resulting mixture was stirred for 4 h at room temperature under an argon atmosphere. The reaction was quenched with H₂O/Ice. The mixture was extracted with EtOAc (5×100 mL). The resulting organic phase was dried over anhydrous Na₂SO₄ and concentrated under reduced pressure. The residue was purified by silica gel column chromatography to afford ethyl 5-amino-1, 2, 4-thiadiazole-3-carboxylate (2.3 g, 23.8% yield) as a solid. LCMS (ESI): m/z [M+H] calc'd for C₅H₇N₃O₂S 174.03; found 173.8.

Step 4. To a solution of ethyl 5-amino-1, 2, 4-thiadiazole-3-carboxylate (5.80 g, 33.49 mmol), MeCN (90.0 mL) and CuBr₂ (11.22 g, 50.23 mmol) at 0° C. was added 2-methyl-2-propylnitrit (6.91 g, 66.98 mmol) dropwise under an argon atmosphere. The mixture was stirred for 30 min. The mixture was then stirred for 4 h at 50° C. The mixture was cooled to 0° C. and quenched with H₂O/Ice. The mixture was extracted with EtOAc (3×100 mL). The resulting organic phase was dried over anhydrous Na₂SO₄ and concentrated under reduced pressure. The residue was purified by silica gel chromatography to afford ethyl 5-bromo-1, 2, 4-thiadiazole-3-carboxylate (6.2 g, 78.1% yield) as a solid. LCMS (ESI): m/z [M+H] calc'd for C₅H₅BrN₂O₂S 236.93; found 237.1.

Step 5. To a solution of (S)-3-(5-bromo-2-(2-(1-methoxyethyl) pyridin-3-yl)-1-(2,2,2-trifluoroethyl)-1H-indol-3-yl)-2,2-dimethylpropan-1-ol (16.60 g, 33.24 mmol), DCM (170.0 mL) and imidazole (5.66 g, 83.10 mmol) at 0° C. was added tert-butyl-chlorodiphenylsilane (11.88 g, 43.21 mmol) dropwise. The resulting mixture was stirred for 3 h at room temperature under an argon atmosphere. The reaction was quenched with H₂O/Ice. The mixture was extracted with EtOAc (3×200 mL). The organic layer was washed with brine (3×100 mL), dried over anhydrous Na₂SO₄ and concentrated under reduced pressure. The residue was purified by silica gel column chromatography to afford(S)-5-bromo-3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indole (22 g, 89.7% yield) as a solid. LCMS (ESI): m/z [M+H] calc'd for C₃₉H₄₄BrF₃N₂O₂Si 737.24; found 737.0.

Step 6. To a solution of (S)-5-bromo-3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indole (28.0 g, 37.95 mmol), toluene (270.0 mL), KOAc (9.31 g, 94.88 mmol) and bis (pinacolato)diboron (19.27 g, 75.90 mmol) at 0° C. was added Pd(dppf)Cl₂·CH₂Cl₂ (6.18 g, 7.59 mmol) in portions. The resulting mixture was stirred for 3 h at 90° C. under an argon atmosphere. The mixture was cooled to room temperature and quenched with H₂O/Ice. The mixture was extracted with EtOAc (3×200 mL). The resulting organic phase was dried over anhydrous Na₂SO₄ and concentrated under reduced pressure. The residue was purified by silica gel column chromatography to afford(S)-3-(3-((tert-butyldiphenylsilyl)oxy)-2,2-dimethylpropyl)-2-(2-(1-methoxyethyl) pyridin-3-yl)-5-(4,4,5,5-tetramethyl-1,3,2-dioxaborolan-2-yl)-1-(2,2,2-trifluoroethyl)-1H-indole (28.2 g, 94.7% yield) as a solid. LCMS (ESI): m/z [M+H] calc'd for C₄₅H₅₆BF₃N₂O₄Si 785.41; found 785.4.

Step 7. To a solution of(S)-3-(3-((tert-butyldiphenylsilyl)oxy)-2,2-dimethylpropyl)-2-(2-(1-methoxyethyl) pyridin-3-yl)-5-(4,4,5,5-tetramethyl-1,3,2-dioxaborolan-2-yl)-1-(2,2,2-trifluoroethyl)-1H-indole (19.60 g, 24.97 mmol), 1,4-dioxane (200 mL), H₂O (40 mL), ethyl 5-bromo-1, 2, 4-thiadiazole-3-carboxylate (5.92 g, 24.97 mmol) and K₃PO₄ (13.25 g, 62.43 mmol) at 0° C. was added Pd(dtbpf)Cl₂ (1.63 g, 2.50 mmol) in portions. The resulting mixture was stirred for 1.5 h at 75° C. under an argon atmosphere. The mixture was cooled to 0° C. and quenched with H₂O/Ice and extracted with EtOAc (3×200 mL). The resulting organic phase was dried over anhydrous Na₂SO₄ and concentrated under reduced pressure. The residue was purified by silica gel column chromatography to afford ethyl(S)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazole-3-carboxylate (14 g, 68.8% yield) as a yellow solid. LCMS (ESI): m/z [M+H] calc'd for C₄₄H₄₉F₃N₄O₄SSi 815.33; found 815.2.

Step 8. To a solution of ethyl(S)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazole-3-carboxylate (13.60 g, 16.69 mmol) and EtOH (140.0 mL) at 0° C. was added NaBH₄ (3.16 g, 83.43 mmol) in portions. The resulting mixture was stirred for 3 h then quenched with H₂O/Ice. The resulting mixture was washed with brine (3×100 mL) and extracted with EtOAc (3×200 mL). The combined organic layers were dried over anhydrous Na₂SO₄ and concentrated under reduced pressure. The residue was purified by silica gel column chromatography to afford(S)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazol-3-ol (9.7 g, 75.2% yield) as a solid. LCMS (ESI): m/z [M+H] calc'd for C₄₂H₄₇F₃N₄O₃SSi 773.32; found 773.3.

Step 9. To a solution of(S)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazol-3-ol (9.70 g, 12.55 mmol), DCM (100.0 mL) and CBr₄ (8.32 g, 25.10 mmol) at 0° C. was added PPh₃ (6.58 g, 25.10 mmol) in DCM (20.0 mL) dropwise. The resulting mixture was stirred for 2 h under an argon atmosphere then quenched with H₂O/Ice. The mixture was extracted with DCM (3×200 mL). The resulting organic phase was dried over anhydrous Na₂SO₄ and concentrated under reduced pressure. The residue was purified by reverse flash chromatography to afford(S)-3-(bromomethyl)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazole (9.5 g, 90.6% yield) as a solid. LCMS (ESI): m/z [M+H] calc'd for C₄₂H₄₆BrF₃N₄O₂SSi 835.23; found 834.9.

Step 10. To a stirred solution of(S)-3-(bromomethyl)-5-(3-(3-((tert-butyldiphenylsilyl)oxy)-2,2-dimethylpropyl)-2-(2-(1-methoxyethyl) pyridin-3-yl)-1-(2,2,2-trifluoroethyl)-1H-indol-5-yl)-1,2,4-thiadiazole (9.40 g, 11.25 mmol), toluene (84.0 mL), DCM (36.0 mL), tert-butyl 2-[(diphenylmethylidene) amino]acetate (3.32 g, 11.25 mmol) and O-Allyl-N-(9-anthracenylmethyl) cinchonidinium bromide (0.68 g, 1.13 mmol) at 0° C. was added 9M KOH aqueous (94.0 mL) dropwise. The resulting mixture was stirred overnight under an argon atmosphere. The mixture was extracted with EtOAc (3×200 mL) and the organic phase was concentrated under reduced pressure. The residue was purified by silica gel column chromatography to afford tert-butyl(S)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl)-2-((diphenylmethylene) amino) propanoate (9 g, 76.2% yield) as a solid. LCMS (ESI): m/z [M+H] calc'd for C₆₁H₆₆F₃N₅O₄SSi 1050.46; found 1050.8.

Step 11. To a solution of tert-butyl(S)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl)-2-((diphenylmethylene) amino) propanoate (8.0 g, 7.62 mmol) and DCM (40.0 mL) at 0° C. solution was added TFA (40.0 mL) dropwise. The resulting mixture was stirred overnight at room temperature then concentrated under reduced pressure. The residue was basified to pH 8 with NaHCO₃. The mixture was extracted with EtOAc (3×200 mL). The organic phase was concentrated under reduced pressure. The residue was purified by reverse flash chromatography to afford(S)-2-amino-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoic acid (5 g, 79.1% yield) as a solid. LCMS (ESI): m/z [M+H] calc'd for C₄₄H₅₀F₃N₅O₄SSi 830.34; found 830.2.

Step 12. To a solution of(S)-2-amino-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoic acid (4.70 g, 5.66 mmol), DCM (50.0 mL) and EtsN (2.86 g, 28.31 mmol) at 0° C. was added (Boc) 2O (1.36 g, 6.23 mmol) dropwise. The resulting mixture was stirred for 3 h at room temperature under an argon atmosphere then concentrated under reduced pressure and purified by reverse flash chromatography to afford(S)-2-((tert-butoxycarbonyl) amino)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoic acid (5 g, 94.9% yield) as a solid. LCMS (ESI): m/z [M+H] calc'd for C₄₉H₅₈F₃N₅O₆SSi 930.39; found 930.3.

Step 13. To a mixture of(S)-2-((tert-butoxycarbonyl) amino)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoic acid (5.30 g, 5.70 mmol), DMF (60.0 mL), methyl 1,2-diazinane-3-carboxylate (1.64 g, 11.40 mmol) and DIPEA (22.09 g, 170.94 mmol) at 0° C. was added HATU (2.82 g, 7.41 mmol) in DMF (5 mL) dropwise. The resulting mixture was stirred for 3 h at room temperature under an argon atmosphere. The reaction was then quenched with H₂O/Ice. The mixture was extracted with EtOAc (3×100 mL) and the organic phase was washed with brine (3×100 mL). The resulting mixture was dried over anhydrous Na₂SO₄ and concentrated under reduced pressure. The residue was purified by silica gel column chromatography to afford methyl(S)-1-((S)-2-((tert-butoxycarbonyl) amino)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoyl) hexahydropyridazine-3-carboxylate (5.6 g) as a solid. LCMS (ESI): m/z [M+H] calc'd for C₅₅H₆₈F₃N₇O₇SSi 1056.47; found 1056.2.

Step 14. A mixture of methyl(S)-1-((S)-2-((tert-butoxycarbonyl) amino)-3-(5-(3-(3-((tert-butyldiphenylsilyl)oxy)-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoyl) hexahydropyridazine-3-carboxylate (5.60 g, 5.30 mmol) and TBAF in THF (56.0 mL) was stirred overnight at 40° C. under an argon atmosphere. The reaction was quenched with sat. NH₄Cl (aq.). The mixture was extracted with EtOAc (3×100 mL) and the organic phase was concentrated under reduced pressure. The residue was purified by reverse flash chromatography to afford(S)-1-((S)-2-((tert-butoxycarbonyl) amino)-3-(5-(3-(3-hydroxy-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoyl) hexahydropyridazine-3-carboxylic acid (4.1 g, 96.2% yield) as a solid. LCMS (ESI): m/z [M+H] calc'd for C₃₈H₄₈F₃N₇O₇S 804.34; found 804.3.

Step 15. To a solution of(S)-1-((S)-2-((tert-butoxycarbonyl) amino)-3-(5-(3-(3-hydroxy-2,2-dimethylpropyl)-2-(2-((S)-1-methoxyethyl) pyridin-3-yl)-1-(2,2,2-trifluoroethyl)-1H-indol-5-yl)-1,2,4-thiadiazol-3-yl) propanoyl) hexahydropyridazine-3-carboxylic acid (4.0 g, 5.0 mmol) and DCM (450.0 mL) at 0° C. were added DIPEA (51.45 g, 398.08 mmol), HOBt (6.72 g, 49.76 mmol) and EDCI (57.23 g, 298.55 mmol) in portions. The resulting mixture was stirred for 16 h at room temperature under an argon atmosphere. The reaction was quenched with H₂O/Ice and extracted with EtOAc (3×30 mL). The organic phase was concentrated under reduced pressure. The residue was purified by silica gel column chromatography to afford tert-butyl ((6³S,4S,Z)-12-(2-((S)-1-methoxyethyl) pyridin-3-yl)-10, 10-dimethyl-5,7-dioxo-11-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl) carbamate (1.7 g, 43.5% yield) as a solid. LCMS (ESI): m/z [M+H] calc'd for C₃₈H₄₆F₃N₇O₆S 786.33; found 786.3.

Step 16. To a solution of ((6³S,4S,Z)-12-(2-((S)-1-methoxyethyl) pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl) carbamate (300.0 mg, 0.38 mmol) and DCM (2.0 mL) at 0° C. was added TFA (1.0 mL) dropwise. The resulting mixture was stirred for 2 h at room temperature then concentrated under reduced pressure. The residue was basified to pH 8 with saturated NaHCO₃(aq.). The mixture was extracted with EtOAc (3×20 mL). The organic phase was concentrated under reduced pressure to afford (6³S,4S,Z)-4-amino-12-(2-((S)-1-methoxyethyl) pyridin-3-yl)-10, 10-dimethyl-11-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-5,7-dione (270 mg, crude) as a solid. LCMS (ESI): m/z [M+H] calc'd for C₃₃H₃₈F₃N₇O₄S 686.27; found 686.1.

Step 17. To a solution of (6³S,4S,Z)-4-amino-12-(2-((S)-1-methoxyethyl) pyridin-3-yl)-10,10-dimethyl-11-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-5,7-dione (160.0 mg, 0.23 mmol), (R)-2-(((1-benzhydrylazetidin-3-yl)oxy) methyl)-3-methylbutanoic acid (123.70 mg, 0.35 mmol) and DMF (2.0 mL) at 0° C. were added DIPEA (603.09 mg, 4.660 mmol) and COMU (119.91 mg, 0.28 mmol) in DMF (0.5 mL). The resulting mixture was stirred for 2 h at room temperature under an argon atmosphere. The reaction was quenched with H₂O/Ice and extracted with EtOAc (3×20 mL). The organic phase was washed with brine (3×10 mL) and concentrated under reduced pressure. The residue was purified by Prep-TLC to afford (2R)-2-(((1-benzhydrylazetidin-3-yl)oxy) methyl)-N-((6³S,4S,Z)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl)-3-methylbutanamide (160 mg, 67.2% yield) as a solid. LCMS (ESI): m/z [M+H] calc'd for C₅₅H₆₃F₃N₈O₆S 1021.46; found 1021.4.

Step 18. To a solution of (2R)-2-(((1-benzhydrylazetidin-3-yl)oxy) methyl)-N-((6³S,4S,Z)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl)-3-methylbutanamide (160.0 mg, 0.16 mmol) and MeOH (5.0 mL) at 0° C. was added (Boc) 2O (85.49 mg, 0.39 mmol) dropwise followed by Pd/C (320.0 mg) in portions. The resulting mixture was stirred overnight at room temperature under a hydrogen atmosphere. The resulting mixture was filtered and the filter cake was washed with EtOAc (3×20 mL). The filtrate was concentrated under reduced pressure. The residue was purified by Prep-TLC to afford tert-butyl 3-((2R)-2-(((6³S,4S,Z)-1²-(2-((S)-1-methoxyethyl) pyridin-3-yl)-10, 10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl) carbamoyl)-3-methylbutoxy) azetidine-1-carboxylate (80 mg, 53.5% yield) as a solid. LCMS (ESI): m/z [M+H] calc'd for C₄₇H₆₁F₃N₈O₈S 955.44; found 955.2.

Step 19. To a solution of tert-butyl 3-((2R)-2-(((6³S,4S,Z)-12-(2-((S)-1-methoxyethyl) pyridin-3-yl)-10, 10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl) carbamoyl)-3-methylbutoxy) azetidine-1-carboxylate (120.0 mg, 0.13 mmol) and DCM (0.80 mL) at 0° C. was added TFA (0.4 mL) dropwise and the resulting mixture was stirred for 2 h at room temperature. The mixture was basified to pH 8 with saturated NaHCO₃(aq.). The mixture was extracted with EtOAc (3×10 mL) and concentrated under reduced pressure. The residue was purified by reverse flash chromatography to afford (2R)-2-((azetidin-3-yloxy) methyl)-N-((6³S,4S,Z)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl)-3-methylbutanamide (40 mg, 37.2% yield) as a solid. LCMS (ESI): m/z [M+H] calc'd for C₄₂H₅₃F₃N₈O₆S 855.38; found 855.3.

Step 20. To a solution of (2R)-2-((azetidin-3-yloxy) methyl)-N-((6³S,4S,Z)-1²-(2-((S)-1-methoxyethyl) pyridin-3-yl)-10, 10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)-pyridazinacycloundecaphane-4-yl)-3-methylbutanamide (32.0 mg, 0.037 mmol),4-(dimethylamino)-4-methylpent-2-ynoic acid (11.62 mg, 0.074 mmol) and DMF (0.50 mL) at 0° C. were added DIPEA (193.49 mg, 1.48 mmol) and COMU (19.23 mg, 0.044 mmol) in DMF (0.1 mL) dropwise. The resulting mixture was stirred for 2 h at room temperature under an argon atmosphere. The reaction was quenched with H₂O/Ice and extracted with EtOAc (3×20 mL). The organic phase was washed with brine (3×10 mL) and concentrated under reduced pressure. The crude product (60 mg) was purified by reverse phase chromatography to afford (2R)-2-(((1-(4-(dimethylamino)-4-methylpent-2-ynoyl) azetidin-3-yl)oxy) methyl)-N-((6³S,4S,Z)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2 (5,3)-thiadiazola-1 (5,3)-indola-6 (1,3)- pyridazinacycloundecaphane-4-yl)-3-methylbutanamide (11.7 mg, 30.9% yield) as a solid. ¹H NMR (400 MHZ, DMSO-d₆) δ 8.80 (dd, J=4.7, 1.8 Hz, 1H), 8.58 (s, 1H), 8.30 (d, J=8.9 Hz, 1H), 7.92 (d, J=8.6 Hz, 1H), 7.84-7.69 (m, 2H), 7.56 (dd, J=7.8, 4.8 Hz, 1H), 5.78 (t, J=8.6 Hz, 2H), 5.10 (d, J=12.1 Hz, 1H), 4.91 (dd, J=16.9, 8.8 Hz, 1H), 4.31 (d, J=6.6 Hz, 6H), 4.05 (dd, J=16.3, 6.6 Hz, 2H), 3.87 (d, J=6.3 Hz, 1H), 3.75 (d, J=11.3 Hz, 1H), 3.61 (d, J=11.0 Hz, 1H), 3.53 (d, J=9.8 Hz, 1H), 3.45 (s, 3H), 3.25 (s, 1H), 3.09 (d, J=10.4 Hz, 1H), 3.01 (d, J=14.5 Hz, 1H), 2.79 (s, 1H), 2.45-2.35 (m, 2H), 2.20 (d, J=5.4 Hz, 6H), 2.15 (d, J=12.0 Hz, 1H), 1.81 (d, 2H), 1.74-1.65 (m, 1H), 1.54 (s, 1H), 1.41-1.30 (m, 9H), 1.24 (s, 1H), 0.94 (s, 3H), 0.90-0.81 (m, 5H), 0.29 (s, 3H). LCMS (ESI): m/z [M+H] calc'd for C₅₀H₆₄F₃N₉O₇S 992.47; found 992.5.

The following table of compounds (Table 3) were prepared using the aforementioned methods or variations thereof, as is known to those of skill in the art.

TABLE 3
Exemplary Compounds Prepared by
Methods of the Present Invention

| | LCMS (ESI): m/z |
|---|---|
| Ex# | [M + H] Found |
| A1 | 944.5 |
| A2 | 888.6 |
| A3 | 903.2 |
| A4 | 876.3 |
| A5 | 903.4 |
| A6 | 945.8 |
| A7 | 1058.1 |
| A8 | 960.8 |
| A9 | 921.7 |
| A10 | 928.8 |
| A11 | 936.4 |
| A12 | 942.5 |
| A13 | 932.5 |
| A14 | 917.5 |
| A15 | 961.5 |
| A16 | 890.6 |
| A17 | 875.4 |
| A18 | 918.5 |
| A19 | 975.6 |
| A20 | 934.5 |
| A21 | 903.3 |
| A22 | 903.6 |
| A23 | 844.6 |
| A24 | 887.6 |
| A25 | 921.4 |
| A26 | 930.4 |
| A27 | 915.6 |
| A28 | 890.6 |
| A29 | 903.5 |
| A30 | 902.7 |
| A31 | 901.4 |
| A32 | 1003.4 |
| A33 | 917.4 |
| A34 | 942.7 |
| A35 | 956.1 |
| A36 | 1005.5 |
| A37 | 917.5 |
| A38 | 959.5 |
| A39 | 942.9 |
| A40 | 915.7 |
| A41 | 917.5 |
| A42 | 888.6 |
| A43 | 942.5 |
| A44 | 904.35 |
| A45 | 846.35 |
| A46 | 982.5 |
| A47 | 960.1 |
| A48 | 905.3 |
| A49 | 981.8 |
| A50 | 916.9 |
| A51 | 946.7 |
| A52 | 916.5 |
| A53 | 921.6 |
| A54 | 947.7 |
| A55 | 916.5 |
| A56 | 904.5 |
| A57 | 930.5 |
| A58 | 895.5 |
| A59 | 944.7 |
| A60 | 931.5 |
| A61 | 847.7 |
| A62 | 871.5 |
| A63 | 930.4 |
| A64 | 893.7 |
| A65 | 874.7 |
| A66 | 876.5 |
| A67 | 970.5 |
| A68 | 915.4 |
| A69 | 927.3 |
| A70 | 876.4 |
| A71 | 933.5 |
| A72 | 1003.5 |
| A73 | 903.6 |
| A74 | 885.5 |
| A75 | 904.7 |
| A76 | 904.5 |
| A77 | 860.6 |
| A78 | 1018.5 |
| A79 | 947.5 |
| A80 | 933.3 |
| A81 | 917.8 |
| A82 | 908.7 |
| A83 | 890.6 |
| A84 | 890.6 |
| A85 | 891.5 |
| A86 | 885.5 |
| A87 | 947.7 |
| A88 | 945.5 |
| A89 | 944.8 |
| A90 | 898 |
| A91 | 890.5 |
| A92 | 903.4 |
| A93 | 901.4 |
| A94 | 881.5 |
| A95 | 987.8 |
| A96 | 945.7 |
| A97 | 931.8 |
| A98 | 943.8 |
| A99 | 849.7 |
| A100 | 942.8 |
| A101 | 893.4 |
| A102 | 904.4 |
| A103 | 947.5 |
| A104 | 931.7 |
| A105 | 915.6 |
| A106 | 902.5 |
| A107 | 945.7 |
| A108 | 902.3 |
| A109 | 906.5 |
| A110 | 793.4 |
| A111 | 946.7 |
| A112 | 905.14 |
| A113 | 849.6 |
| A114 | 888.4 |
| A115 | 888.4 |
| A116 | 986.5 |
| A117 | 879.5 |
| A118 | 881.6 |
| A119 | 874.5 |
| A120 | 929.7 |
| A121 | 943.6 |
| A122 | 888.8 |
| A123 | 959.3 |
| A124 | 924.6 |
| A125 | 962.5 |
| A126 | 986.5 |
| A127 | 946.6 |
| A128 | 903.4 |
| A129 | 1001.2 |
| A130 | 930.5 |
| A131 | 959.5 |
| A132 | 940.5 |
| A133 | 969.7 |
| A134 | 990.5 |
| A135 | 933.5 |
| A136 | 875.4 |
| A137 | 889.5 |
| A138 | 929.4 |
| A139 | 930.4 |
| A140 | 943.7 |
| A141 | 943.8 |
| A142 | 901.7 |
| A143 | 852.3 |
| A144 | 886.7 |
| A145 | 916.7 |
| A146 | 963.4 |
| A147 | 864.4 |
| A148 | 885.5 |
| A149 | 947.5 |
| A150 | 902.7 |
| A151 | 904.5 |
| A152 | 885.5 |
| A153 | 949.6 |
| A154 | 907.5 |
| A155 | 944.5 |
| A156 | 943.7 |
| A157 | 943.5 |
| A158 | 904.5 |
| A159 | 904.5 |
| A160 | 858.5 |
| A161 | 947.4 |
| A162 | 973.5 |
| A163 | 989.4 |
| A164 | 947.5 |
| A165 | 960.3 |
| A166 | 907.7 |
| A167 | 918.5 |
| A168 | 907.4 |
| A169 | 859.4 |
| A170 | 874.3 |
| A171 | 867.5 |
| A172 | 883.6 |
| A173 | 900.5 |
| A174 | 848.4 |
| A175 | 959.5 |
| A176 | 944.5 |
| A177 | 935.4 |
| A178 | 930.4 |
| A179 | 849.5 |
| A180 | 849.5 |
| A181 | 918.8 |
| A182 | 1002.2 |
| A183 | 961.5 |
| A184 | 1003.5 |
| A185 | 928.5 |
| A186 | 992.5 |
| A187 | 945.5 |
| A188 | 960.4 |
| A189 | 1000.6 |
| A190 | 934.3 |
| A191 | 985.5 |
| A192 | 971.5 |
| A193 | 871.6 |
| A194 | 918.5 |
| A195 | 927.4 |
| A196 | 972.3 |
| A197 | 896.4 |
| A198 | 916.6 |
| A199 | 916.6 |
| A200 | 987.4 |
| A201 | 993.7 |
| A202 | 896.4 |
| A203 | 987.6 |
| A204 | 987.6 |
| A205 | 933.5 |
| A206 | 930.5 |
| A207 | 930.5 |
| A208 | 958.8 |
| A209 | 1015.7 |
| A210 | 906.5 |
| A211 | 905.5 |
| A212 | 901.3 |
| A213 | 969.5 |
| A214 | 933.6 |
| A215 | 890.3 |
| A216 | 916.4 |
| A217 | 930.5 |
| A218 | 983.5 |
| A219 | 917.4 |
| A220 | 996.5 |
| A221 | 915.4 |
| A222 | 930.05 |
| A223 | 920.01 |
| A224 | 983.5 |
| A225 | 982.7 |
| A226 | 873.7 |
| A227 | 887.7 |
| A228 | 911 |
| A229 | 896.7 |
| A230 | 905.5 |
| A231 | 912.4 |
| A232 | 849.4 |
| A233 | 905.4 |
| A234 | 1053.3 |
| A235 | 835.5 |
| A236 | 882.3 |
| A237 | 1015.4 |
| A238 | 971.5 |
| A239 | 869.4 |
| A240 | 884.4 |
| A241 | 987.4 |
| A242 | 877.4 |
| A243 | 863.5 |
| A244 | 1029.6 |
| A245 | 1029.5 |
| A246 | 501.4 |
| A247 | 985.5 |
| A248 | 896.4 |
| A249 | 984.5 |
| A250 | 927.4 |
| A251 | 985.4 |
| A252 | 927.3 |
| A253 | 990.4 |
| A254 | 933.3 |
| A255 | 930.7 |
| A256 | 890.4 |
| A257 | 947.4 |
| A258 | 990.3 |
| A259 | 933.3 |
| A260 | 918.4 |
| A261 | 975.5 |
| A262 | 919.4 |
| A263 | 976.5 |
| A264 | 990.5 |
| A265 | 933.5 |
| A266 | 945.5 |
| A267 | 987.5 |
| A268 | 973.5 |
| A269 | 487.3 |
| A270 | 959.4 |
| A271 | 861.3 |
| A272 | 847.6 |
| A273 | 861.6 |
| A274 | 930.5 |
| A275 | 916.3 |
| A276 | 892.8 |
| A277 | 895.7 |
| A278 | 879.5 |
| A279 | 895.4 |
| A280 | 954.6 |
| A281 | 916.6 |
| A282 | 952.5 |
| A283 | 931.4 |
| A284 | 889.5 |
| A285 | 839.4 |
| A286 | 877.4 |
| A287 | 894.7 |
| A288 | 879.3 |
| A289 | 908.7 |
| A290 | 971.6 |
| A291 | 978.5 |
| A292 | 987.5 |
| A293 | 921.4 |
| A294 | 902.8 |
| A295 | 916.4 |
| A296 | 930.8 |
| A297 | 938.4 |
| A298 | 952.6 |
| A299 | 966.7 |
| A300 | 919.6 |
| A301 | 907.7 |
| A302 | 895.8 |
| A303 | 895.4 |
| A304 | 897.5 |
| A305 | 968.6 |
| A306 | 968.7 |
| A307 | 952.6 |
| A308 | 952.7 |
| A309 | 933.6 |
| A310 | 926.8 |
| A311 | 944.7 |
| A312 | 897.5 |
| A313 | 976.5 |
| A314 | 893.3 |
| A316 | 930.5 |
| A317 | 982.6 |
| A318 | 883.5 |
| A319 | 916.5 |
| A320 | 886.6 |
| A321 | 900.6 |
| A322 | 1004.4 |
| A323 | 976.5 |
| A324 | 969.5 |
| A325 | 1033.7 |
| A326 | 997.5 |
| A327 | 906.6 |
| A328 | 986.7 |
| A329 | 890.5 |
| A330 | 895.6 |
| A331 | 879.5 |
| A332 | 942.5 |
| A333 | 919.6 |
| A334 | 930.7 |
| A335 | 924.5 |
| A336 | 927.4 |
| A337 | 501.4 |
| A338 | 919.4 |
| A339 | 971.6 |
| A340 | 895.7 |
| A341 | 938.5 |
| A342 | 906.8 |
| A343 | 992.4 |
| A344 | 901.40 |
| A345 | 897.40 |
| A346 | 942.50 |
| A347 | 896.30 |
| A348 | 1,002.30 |
| A349 | 924.6 |
| A350 | 968.2 |
| A351 | 997.6 |
| A352 | 952.7 |
| A353 | 966.3 |
| A354 | 966.2 |
| A355 | 855.6 |
| A356 | 910.4 |
| A357 | 1091.0 |
| A358 | 902.5 |
| A359 | 904.7 |
| A360 | 924.5 |
| A361 | 967.5 |
| A362 | 936.6 |
| A363 | 961.4 |
| A364 | 946.5 |
| A365 | 995.6 |
| A366 | 921.5 |
| A367 | 950.6 |
| A368 | 924.5 |
| A369 | 924.5 |
| A370 | 1087.3 |
| A371 | 902.5 |
| A372 | 992.1 |
| A373 | 902.6 |
| A374 | 1,034.40 |
| A375 | 993.50 |
| A376 | 984.40 |
| A377 | 939.50 |
| A378 | 897.50 |
| A379 | 1005.6 |
| A380 | 949.4 |
| A381 | 921.5 |
| A382 | 938.5 |
| A383 | 938.5 |
| A384 | 924.5 |
| A385 | 938.3 |
| A386 | 938.4 |
| A387 | 938.5 |
| A388 | 938.5 |
| A389 | 936.5 |
| A390 | 924.5 |
| A391 | 1011.6 |
| A392 | 958.0 |
| A393 | 922.4 |
| A394 | 950.3 |
| A395 | 936.5 |
| A396 | 924.4 |
| A397 | 924.7 |
| A398 | 914.6 |
| A399 | 902.5 |
| A400 | 956.6 |
| A401 | 955.9 |
| A402 | 940.8 |
| A403 | 948.40 |
| A404 | 980.40 |
| A405 | 911.50 |
| A406 | 938.7 |
| A407 | 922.5 |
| A408 | 922.5 |
| A409 | 922.6 |
| A410 | 1046.9 |
| A411 | 990.7 |
| A412 | 912.7 |
| A413 | 914.7 |
| A414 | 983.5 |
| A415 | 950.5 |
| A416 | 924.6 |
| A417 | 936.7 |
| A418 | 938.5 |
| A419 | 922.3 |
| A420 | 936.7 |
| A421 | 924.5 |
| A422 | 910.5 |
| A423 | 1028.9 |
| A424 | 934.6 |
| A425 | 1012.5 |
| A426 | 912.5 |
| A427 | 895.30 |
| A428 | 951.30 |
| A429 | 911.50 |
| A430 | 938.5 |
| A431 | 952.3 |
| A432 | 924.2 |
| A433 | 924.2 |
| A434 | 924.7 |
| A435 | 910.5 |
| A436 | 924.6 |
| A437 | 912.5 |
| A438 | 898.5 |
| A439 | 1016.6 |
| A440 | 990.5 |
| A441 | 939.6 |
| A442 | 889.2 |
| A443 | 913.5 |
| A444 | 913.5 |
| A445 | 890.1 |
| A446 | 957.6 |
| A447 | 880.5 |
| A448 | 888.4 |
| A449 | 920.5 |
| A450 | 897.2 |
| A451 | 902.6 |
| A452 | 888.4 |
| A453 | 901.3 |
| A454 | 1044.6 |
| A455 | 949.2 |
| A456 | 1059.5 |
| A457 | 916.7 |
| A458 | 916.2 |
| A459 | 902.3 |
| A460 | 874.5 |
| A461 | 957.9 |
| A462 | 854.3 |
| A463 | 910.7 |
| A464 | 964.5 |
| A465 | 964.5 |
| A466 | 1033.7 |
| A467 | 874.5 |
| A468 | 879.6 |
| A469 | 902.6 |
| A470 | 900.6 |
| A471 | 900.6 |
| A472 | 964.6 |
| A473 | 854.3 |
| A474 | 914.4 |
| A475 | 914.40 |
| A476 | 983.6 |
| A477 | 907.5 |
| A478 | 900.5 |
| A479 | 993.6 |
| A480 | 902.2 |
| A481 | 930.6 |
| A482 | 974.6 |
| A483 | 847.5 |
| A484 | 847.4 |
| A485 | 938.4 |
| A486 | 893.5 |
| A487 | 900.6 |
| A488 | 888.5 |
| A489 | 962.6 |
| A490 | 896.5 |
| A491 | 910.2 |
| A492 | 910.5 |
| A493 | 910.5 |
| A494 | 886.3 |
| A495 | 907.5 |
| A496 | 900.4 |
| A497 | 902.7 |
| A498 | 902.6 |
| A499 | 998.7 |
| A500 | 897.5 |
| A501 | 906.3 |
| A502 | 949.2 |
| A503 | 920.6 |
| A504 | 920.8 |
| A505 | 920.0 |
| A506 | 879.3 |
| A507 | 897.4 |
| A508 | 942.4 |
| A509 | 942.4 |
| A510 | 899.3 |
| A511 | 919.5 |
| A512 | 900.5 |
| A513 | 983.7 |
| A514 | 983.7 |
| A515 | 985.7 |
| A516 | 985.7 |
| A517 | 892.6 |
| A518 | 920.6 |
| A519 | 920.4 |
| A520 | 990.6 |
| A521 | 990.6 |
| A522 | 945.3 |
| A523 | 945.4 |
| A524 | 914.5 |
| A525 | 914.4 |
| A526 | 1005.4 |
| A527 | 972.4 |
| A528 | 985.4 |
| A529 | 987.4 |
| A530 | 898.5 |
| A531 | 912.3 |
| A532 | 1013.6 |
| A533 | 999.3 |
| A534 | 983.4 |
| A535 | 919.5 |
| A536 | 961.7 |
| A537 | 961.6 |
| A532 | 1013.6 |
| A533 | 999.3 |
| A534 | 983.4 |
| A535 | 919.5 |
| A536 | 961.7 |
| A537 | 961.6 |
| A538 | 999.7 |
| A539 | 960.4 |
| A540 | 1050.2 |
| A541 | 1078.7 |
| A542 | 1155.7 |
| A543 | 952.6 |
| A544 | 952.6 |
| A545 | 981.2 |
| A546 | 922.3 |
| A547 | 922.5 |
| A548 | 950.5 |
| A549 | 997.4 |
| A550 | 1025.6 |
| A551 | 997.5 |
| A552 | 950.4 |
| A553 | 974.6 |
| A554 | 957.5 |
| A555 | 1054.4 |
| A556 | 972.5 |
| A557 | 952.4 |
| A558 | 959.6 |
| A559 | 1011.7 |
| A560 | 1011.6 |
| A561 | 922.5 |
| A562 | 954.3 |
| A563 | 978.5 |
| A564 | 982.5 |
| A565 | 1039.3 |
| A566 | 1039.4 |
| A567 | 1039.4 |
| A568 | 1039.3 |
| A569 | 997.4 |
| A570 | 974.7 |
| A571 | 1038.5 |
| A572 | 1056.5 |
| A573 | 1063.5 |
| A574 | 990.6 |
| A575 | 1004.5 |
| A576 | 991.5 |
| A577 | 1062.3 |
| A578 | 1048.3 |
| A579 | 963.3 |
| A580 | 966.6 |
| A581 | 966.6 |
| A582 | 953.5 |
| A583 | 938.5 |
| A584 | 944.7 |
| A585 | 944.6 |
| A586 | 950.6 |
| A587 | 962.6 |
| A588 | 938.5 |
| A589 | 1016.6 |
| A590 | 992.5 |
| A591 | 1008.7 |
| A592 | 997.5 |
| A593 | 1020.5 |
| A594 | 1020.6 |
| A595 | 1000.6 |
| A596 | 966.7 |
| A597 | 946.6 |
| A598 | 979.6 |
| A599 | 958.7 |
| A600 | 887.4 |
| A601 | 985.5 |
| A602 | 1036.5 |
| A603 | 989.9 |
| A604 | 1006.1 |
| A605 | 1059.7 |
| A606 | 1032.6 |
| A607 | 1028.1 |
| A608 | 1027.1 |
| A609 | 966.6 |
| A610 | 945.6 |
| A611 | 1021.5 |
| A612 | 920.6 |
| A613 | 956.1 |
| A614 | 992.5 |
| A615 | 1010.5 |
| A616 | 1010.5 |
| A617 | 990.5 |
| A618 | 950.7 |
| A619 | 950.4 |
| A620 | 938.1 |
| A621 | 966.1 |
| A622 | 982.6 |
| A623 | 999.6 |
| A624 | 1019.5 |
| A625 | 1019.5 |
| A626 | 913.5 |
| A627 | 913.4 |
| A628 | 1046.5 |
| A629 | 952.2 |
| A630 | 967.6 |
| A631 | 942.5 |
| A632 | 942.6 |
| A633 | 938.6 |
| A634 | 938.6 |
| A635 | 978.6 |
| A636 | 916.6 |
| A637 | 909.5 |
| A638 | 909.5 |
| A639 | 928.3 |
| A640 | 942.0 |
| A641 | 942.6 |
| A642 | 1047.5 |
| A643 | 966.6 |
| A644 | 944.4 |
| A645 | 986.5 |
| A646 | 937.5 |
| A647 | 1012.5 |
| A648 | 944.5 |
| A649 | 921.6 |
| A650 | 1007.6 |
| A651 | 964.2 |
| A652 | 1903.1 |
| A653 | 938.6 |
| A654 | 938.6 |
| A655 | 934.4 |
| A656 | 1005.5 |
| A657 | 960.7 |
| A658 | 1003.3 |
| A659 | 974.6 |
| A660 | 925.5 |
| A661 | 936.6 |
| A662 | 936.6 |
| A663 | 935.5 |
| A664 | 1047.5 |
| A665 | 1035.5 |
| A666 | 1035.2 |
| A667 | 936.3 |
| A668 | 965.6 |
| A669 | 1049.7 |
| A670 | 1047.7 |
| A671 | 964.6 |
| A672 | 997.9 |
| A673 | 1015.3 |
| A674 | 975.5 |
| A675 | 921.5 |
| A676 | 966.5 |
| A677 | 966.4 |
| A678 | 950.5 |
| A679 | 990.2 |
| A680 | 943.6 |
| A681 | 980.6 |
| A682 | 968.6 |
| A683 | 1000.6 |
| A684 | 915.0 |
| A685 | 977.6 |
| A686 | 922.5 |
| A687 | 936.4 |
| A688 | 936.5 |
| A689 | 1036.9 |
| A690 | 979.6 |
| A691 | 923.5 |
| A692 | 962.5 |
| A693 | 906.5 |
| A694 | 896.1 |
| A695 | 980.7 |
| A696 | 978.4 |
| A697 | 978.3 |
| A698 | 964.3 |
| A699 | 1003.3 |
| A700 | 927.2 |
| A701 | 993.7 |
| A702 | 950.6 |
| A703 | 985.5 |
| A704 | 938.6 |
| A705 | 925.5 |
| A706 | 952.5 |
| A707 | 1016.5 |
| A708 | 903.2 |
| A709 | 981.5 |
| A710 | 967.5 |
| A711 | 964.5 |
| A712 | 937.9 |
| A713 | 939.9 |
| A714 | 952.5 |
| A715 | 937.4 |
| A716 | 893.5 |
| A717 | 924.6 |
| A718 | 936.6 |
| A719 | 950.5 |
| A720 | 935.3 |
| A721 | 937.3 |
| A722 | 929.4 |
| A723 | 910.4 |
| A724 | 985.4 |
| A725 | 919.3 |
| A726 | 1010.4 |
| A727 | 1025.5 |
| A728 | 924.6 |
| A729 | 993.5 |
| A730 | 993.2 |
| A731 | 922.7 |
| A732 | 928.6 |
| A733 | 997.6 |
| A734 | 936.5 |
| A735 | 952.5 |
| A736 | 922.2 |
| A737 | 922.1 |
| A738 | 924.5 |
| A739 | 924.5 |
| A730 | 993.2 |
| A731 | 922.7 |
| A732 | 928.6 |
| A733 | 997.6 |
| A734 | 936.5 |
| A735 | 952.5 |
| A736 | 922.2 |
| A737 | 922.1 |
| A738 | 924.5 |
| A739 | 924.5 |
| A740 | 895.5 |
| A741 | 895.4 |

Blank = not determined

### Matched Pair Analysis

FIGS. 1A-1B compare the potency in two different cell-based assays of compounds of Formula BB of the present invention (points on the right) and corresponding compounds of Formula AA (points on the left) wherein a H is replaced with(S) Me. The y axes represent pERK EC50 (FIG. 1A) or CTG IC50 (FIG. 1B) as measured in an H358 cell line. Assay protocols are below. The linked points represent a matched pair that differs only between H and(S) Me substitution. Each compound of Formula BB demonstrated reduced potency in cell assays compared to the corresponding compound of Formula AA.

### Biological Assays

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H₃₅₈ cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 L tris buffered saline (TBS) and fixed for 15 minutes with 150 μL 4% paraformaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (PERK, CST-4370, Cell Signaling Technology) was diluted 1:200 in blocking buffer, and 50 μL was added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li—COR Odyssey CLx Imager. Analysis of raw data was carried out in Excel (Microsoft) and Prism (GraphPad). Signal was plotted vs. the decadal logarithm of compound concentration, and IC50 was determined by fitting a 4-parameter sigmoidal concentration response model.

The following compounds exhibited a pERK EC50 of under 5 μM (H358 KRAS G12C): A48, A15, A272, A174, A163, A453, A447, A279, A240, A214, A225, A136, A226, A219, A228, A21, A12, A78, A424, A219, A378, A224, A4, A53, A187, A218, A213, A314, A220, A208, A24, A9, A126, A345, A46, A203, A210, A184, A469, A366, A113, A328, A693, A639, A364, A100, A249, A486, A307, A347, A33, A210, A192, A285, A468, A185, A612, A109, A284, A200, A2, A6, A606, A325, A139, A496, A393, A561, A125, A494, A54 7, A215, A258, A195, A259, A212, A637, A53, A63, A68, A178, A189, A205, A78, A254, A690, A563, A14, A1 9, A92, A576, A278, A331, A42, A67, A209, A350, A562, A652, A703, A623, A191, A241, A199, A193, A478, A251, A177, A222, A23, A59, A26, A211, A106, A279, A120, A7, A134, A521, A116, A467, A694, A729, A151, A110, A277, A340, A221, A723, A13, A442, A611, A50, A190, A553, A696, A211, A303, A613, A37, A146,A 666, A688, A216, A390, A548, A238, A160, A183, A164, A451, A481, A524, A1, A186, A37, A635, A71, A269, A289, A489, A400, A731, A497, A568, A274, A253, A471, A720, A241, A179, A180, A426, A117, A363, A71 6, A423, A217, A708, A227, A3, A12, A8, A381, A84, A408, A85, A171, A263, A473, A258, A564, A118, A103, A565, A641, A655, A47, A11, A392, A169, A487, A640, A206, A449, A358, A192, A148, A4, A41, A5, A18, A3 01, A10, A65, A554, A159, A264, A99, A79, A142, A143, A25, A98, A80, A101, A730, A212, A359, A61, A441, A283, A413, A717, A145, A182, A62, A181, A233, A232, A634, A495, A34, A251, A539, A632, A54, A327, A3 7, A196, A607, A645, A35, A214, A225, A638, A40, A52, A268, A448, A575, A176, A593, A15, A17, A94, A17 0, A713, A93, A402, A64, A261, A399, A422, A214, A225, A625, A31, A119, A135, A281, A676, A709, A81,A 32, A633, A39, A646, A662, A124, A732, A320, A81, A187, A354, A45, A570, A165, A66, A20, A455, A431,A 270, A250, A457, A153, A404, A710, A541, A127, A373, A369, A557, A349, A598, A618, A60, A636, A499,A 87, A156, A680, A477, A406, A330, A202, A535, A617, A737, A201, A302, A722, A209, A374, A631, A29, A5 55, A420, A380, A111, A306, A173, A628, A672, A51, A167, A588, A512, A194, A282, A412, A701, A583, A3 96, A678, A649, A27, A204, A626, A257, A614, A409, A172, A372, A353, A58, A728, A74, A619, A144, A183, A538, A445, A531, A360, A361, A459, A536, A344, A267, A574, A677, A530, A415, A30, A73, A152, A490, A702, A714, A483, A567, A43, A310, A319, A86, A321, A656, A739, A115, A130, A155, A608, A648, A168,A 485, A738, A129, A650, A715, A488, A147, A121, A470, A115, A133, A510, A421, A309, A335, A387, A386, A734, A95, A430, A604, A458, A592, A384, A664, A197, A725, A89, A83, A586, A622, A305, A498, A668, A4 27, A630, A158, A644, A735, A70, A683, A352, A341, A719, A674, A70, A44, A501, A438, A698, A377, A417, A154, A433, A104, A184, A603, A280, A712, A237, A105, A394, A605, A517, A704, A566, A77, A356, A454, A600, A643, A112, A569, A529, A247, A463, A437, A718, A472, A461, A558, A48, A671, A395, A670, A681, A687, A382, A82, A686, A342, A436, A296, A16, A545, A533, A416, A149, A207, A371, A596, A675, A132, A419, A56, A579, A733, A573, A707, A597, A697, A75, A653, A362, A615, A332, A69, A162, A128, A432, A6 54, A22, A397, A526, A582, A418, A91, A260, A97, A191, A55, A581, A375, A522, A108, A367, A610, A552, A571, A57, A543, A661, A138, A196, A246, A337, A446, A265, A96, A509, A123, A627, A651, A682, A157,A 572, A624, A691, A532, A462, A580, A695, A186, A316, A540, A590, A665, A244, A166, A587, A629, A595, A518, A519, A131, A502, A726, A452, A141, A181, A262, A338, A155, A389, A124, A275, A414, A546, A67 9, A425, A669, A28, A520, A88, A131, A589, A621, A182, A297, A594, A283, A194, A250, A336, A706, A252, A440, A107, A724, A525, A388, A175, A300, A333, A659, A346, A150, A476, A368, A528, A503, A504, A50 5, A684, A76, A736, A551, A383, A491, A492, A493, A410, A316, A295, A559, A511, A38, A140, A663, A334, A700, A692, A348, A584, A513, A657, A328, A515, A317, A135, A660, A351, A544, A281, A685, A602, A556, A385, A326, A464, A465, A403, A133, A299, A667, A255, A334, A256, A585, A642, A133, A443, A435, A5 60, A444, A439, A324, A120, A407, A527, A245, A370, A537, A247, A474, A475, A705, A323, A112, A298,A 609, A673, A292, A599, A132, A145, A266, A601, A466, A549, A379, A727, A167, A711, A75, A76, A121, A3 57, A620, A316, A479, A290, A339, A322, A376, A456, A391, A291, A550, A343, A721, A689, A411, A578,A 616, A534, A365, A658, A699, A577, A647, A591, A542, A279, A294.

### Determination of Cell Viability in RAS Mutant Cancer Cell Lines Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 UL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-binding Domain (BRAF^{RBD}) Interaction with K-Ras by Compounds of the Invention (also called a FRET assay or an MOA assay)

Note—The following protocol describes a procedure for monitoring disruption of K-Ras G12C(GMP-PNP) binding to BRAF^{RBD} by a compound of the invention. This protocol may also be executed substituting other Ras proteins or nucleotides.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} were combined in a 384-well assay plate at final concentrations of 25 μM, 12.5 nM and 50 nM, respectively. Compound was present in plate wells as a 10-point 3-fold dilution series starting at a final concentration of 30 μM. After incubation at 25° C. for 3 hours, a mixture of Anti-His Eu—W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras: RAF complex were identified as those eliciting a decrease in the TR-FRET ratio relative to DMSO control wells.

TABLE 4
Biological Assay Data for Representative Compounds
of the Present Invention

| | H358 pERK | H358 Cell | | |
|---|---|---|---|---|
| | (K-Ras | Viability | FRET | FRET |
| | G12C) | (K-Ras G12C) | (K-Ras G12C) | (K-Ras G13C) |
| Ex# | EC50, uM | IC50, uM | IC50, uM | IC50, uM |
| A208 | 0.013 | 0.004 | 0.023 | 7.48 |
| A193 | 0.067 | 0.054 | 0.295 | 10.2 |
| A186 | 0.013 | 0.005 | 0.038 | 0.94 |
| A89a | 0.003 | 0.0008 | 0.009 | 0.82 |
| A89b | 0.028 | 0.011 | 0.034 | 0.58 |

TABLE 5
Additional H358 Cell Viability assay data (K-Ras G12C, IC50, uM):
*Key:
++++: IC50 ≥ 10 uM
+++ 1 uM > IC50 ≥ 0.1 uM
++: 0.1 uM > IC50 ≥ 0.01 uM
+: IC50 < 0.01 uM

| IC50* | Examples |
|---|---|
| + | A104, A107, A108, A112, A120, A121, A123, |
| | A124, A128, A131, A131, A132, A133, |
| | A133, A135, A138, A140, A141, A145, A150, |
| | A155, A157, A162, A166, A167, A175, |
| | A181, A182, A183, A186, A191, A194, A196, |
| | A237, A244, A245, A246, A247, A250, A255, |
| | A256, A260, A266, A275, A28, A281, A283, |
| | A290, A291, A292, A295, A296, A298, |
| | A299, A300, A309, A316, A316, A316, A317, |
| | A322, A323, A324, A326, A328, A332, |
| | A333, A334, A334, A336, A337, A339, A343, |
| | A344, A346, A348, A351, A352, A356, |
| | A357, A365, A368, A370, A371, A375, A376, |
| | A377, A379, A38, A380, A382, A383, A384, |
| | A385, A388, A389, A391, A394, A395, A396, |
| | A397, A403, A407, A410, A411, A414, |
| | A416, A419, A425, A427, A435, A439, A440, |
| | A443, A444, A445, A446, A452, A454, |
| | A456, A461, A462, A463, A464, A465, A466, |
| | A472, A474, A475, A476, A479, A483, |
| | A485, A488, A491, A492, A493, A502, A503, |
| | A504, A505, A509, A511, A513, A515, A517, |
| | A518, A519, A520, A522, A525, A526, A527, |
| | A528, A532, A533, A534, A537, A540, |
| | A542, A543, A544, A546, A549, A55, A550, |
| | A551, A552, A556, A559, A56, A560, A566, |
| | A567, A57, A571, A572, A577, A578, A579, |
| | A581, A584, A585, A587, A589, A590, |
| | A591, A592, A594, A595, A596, A597, A599, |
| | A600, A601, A602, A604, A609, A610, |
| | A615, A616, A620, A621, A624, A627, A629, |
| | A631, A636, A642, A643, A644, A647, A657, |
| | A658, A659, A660, A661, A663, A665, A667, |
| | A673, A674, A675, A677, A684, A685, |
| | A686, A689, A691, A692, A699, A700, A704, |
| | A705, A706, A707, A711, A712, A714, |
| | A718, A721, A724, A726, A727, A733, A735, |
| | A736, A738, A75, A76, A76, A88, A91, A96 |
| ++ | A10, A105, A111, A112, A115, A115, A119, |
| | A12, A121, A124, A129, A130, A132, A133, |
| | A135, A143, A144, A145, A147, A149, A15, |
| | A152, A154, A155, A156, A158, A16, |
| | A165, A167, A168, A172, A173, A176, A181, |
| | A182, A184, A187, A194, A197, A20, A201, |
| | A202, A204, A207, A209, A212, A214, A214, |
| | A22, A225, A225, A232, A233, A238, |
| | A241, A247, A250, A251, A252, A257, A261, |
| | A262, A264, A265, A267, A268, A269, |
| | A27, A270, A280, A281, A282, A283, A297, |
| | A30, A302, A305, A306, A31, A310, A319, |
| | A320, A321, A327, A330, A335, A338, A34, |
| | A341, A342, A349, A35, A353, A354, A358, |
| | A359, A360, A361, A362, A363, A367, A369, |
| | A37, A372, A373, A374, A381, A386, |
| | A387, A39, A392, A393, A399, A40, A400, A402, |
| | A404, A406, A408, A409, A41, A412, |
| | A413, A415, A417, A418, A420, A421, A422, |
| | A423, A426, A43, A430, A431, A432, |
| | A433, A436, A437, A438, A44, A441, A442, |
| | A448, A449, A45, A451, A455, A457, A458, |
| | A459, A467, A468, A47, A470, A471, A473, |
| | A477, A478, A48, A481, A487, A489, A490, |
| | A495, A497, A498, A499, A5, A501, A51, |
| | A510, A512, A52, A524, A529, A530, A531, |
| | A535, A536, A538, A54, A541, A545, A555, |
| | A558, A564, A565, A568, A569, A573, |
| | A574, A575, A58, A580, A582, A583, A586, |
| | A588, A598, A60, A603, A605, A607, A608, |
| | A61, A611, A613, A614, A617, A618, A619, |
| | A62, A622, A623, A625, A626, A628, A630, |
| | A633, A634, A637, A638, A64, A640, A641, |
| | A645, A646, A648, A649, A650, A651, |
| | A653, A654, A655, A656, A66, A662, A664, |
| | A666, A668, A669, A670, A671, A672, |
| | A676, A678, A679, A680, A681, A682, A683, |
| | A687, A69, A694, A695, A697, A698, A70, |
| | A70, A701, A702, A703, A708, A709, A710, |
| | A713, A715, A716, A717, A719, A720, |
| | A722, A723, A725, A728, A73, A730, A732, |
| | A734, A737, A739, A74, A75, A77, A79, |
| | A81, A82, A83, A86, A87, A89, A93, A94, |
| | A95, A97, A98, A99 |
| +++ | A1, A100, A101, A103, A106, A109, A11, A110, |
| | A113, A114, A116, A117, A118, A120, |
| | A127, A13, A134, A139, A14, A142, A144, |
| | A146, A148, A151, A153, A159, A160, A164, |
| | A169, A17, A170, A171, A177, A18, A183, |
| | A185, A186, A19, A190, A191, A192, A192, |
| | A193, A195, A196, A199, A200, A205, A206, |
| | A209, A210, A211, A211, A212, A215, |
| | A216, A217, A219, A219, A221, A222, A226, |
| | A227, A23, A241, A249, A25, A251, |
| | A253, A254, A258, A258, A259, A26, A263, |
| | A274, A277, A278, A279, A284, A289, A29, |
| | A3, A301, A303, A304, A304, A304, A304, |
| | A308, A314, A32, A325, A328, A329, A33, |
| | A331, A340, A345, A347, A350, A355, A364, |
| | A366, A37, A37, A378, A390, A398, A4, |
| | A401, A42, A424, A447, A453, A469, A482, |
| | A484, A486, A494, A496, A50, A506, A507, |
| | A508, A514, A521, A523, A53, A53, A539, |
| | A547, A548, A553, A554, A557, A561, |
| | A562, A563, A570, A576, A59, A593, A6, |
| | A606, A612, A63, A632, A635, A639, A65, |
| | A652, A67, A68, A688, A690, A693, A696, |
| | A7, A71, A729, A731, A78, A8, A80, A81, A84, |
| | A85, A9, A92 |
| ++++ | A102, A12, A122, A125, A126, A136, A137, |
| | A148, A15, A161, A163, A174, A178, A179, |
| | A180, A184, A187, A188, A189, A198, A2, |
| | A203, A208, A21, A210, A210, A210, A211, |
| | A211, A213, A213, A214, A214, A215, A216, |
| | A217, A218, A220, A220, A221, A222, |
| | A223, A223, A224, A224, A225, A225, A228, |
| | A229, A230, A231, A231, A231, A234, |
| | A235, A236, A239, A239, A24, A240, A242, |
| | A243, A248, A261, A271, A272, A273, |
| | A276, A279, A279, A283, A285, A286, A287, |
| | A288, A293, A307, A311, A312, A313, A318, |
| | A318, A32, A4, A405, A428, A429, A450, |
| | A46, A460, A48, A480, A49, A500, A516, |
| | A72, A78, A90 |

Additional Ras-Raf disruption/FRET/MOA assay data (IC50, uM):
*Key:
+++++: IC50 ≥ 10 uM
++++: 10 uM > IC50 ≥ 1 uM
+++: 1 uM > IC50 ≥ 0.1 uM
++: 0.1 uM > IC50 ≥ 0.01 uM
+: IC50 < 0.01 uM

TABLE 6
KRAS G12S FRET data

| IC50* | Examples |
|---|---|
| + | none |
| ++ | A028, A075, A076, A076, A087, A112, A145, A155, A167, |
| | A181, A183, A184, A194, A233, A255, A256, A260, A262, |
| | A264, A265, A266, A268, A270, A275, A292, A294, A295, |
| | A298, A299, A300, A319, A333, A334, A334, A338, A367, |
| | A382, A383, A385, A388, A389, A418, A433, A464, A465, |
| | A479, A491, A492, A493, A527, A537, A542, A577, A596, |
| | A598, A602, A609, A621, A629, A664, A665, A679, A712, |
| | A734, A736 |
| +++ | A004, A005, A012, A015, A016, A022, A027, A029, A032, |
| | A034, A037, A037, A041, A043, A044, A047, A048, A051, |
| | A052, A054, A055, A056, A057, A058, A064, A070, A070, |
| | A072, A074, A077, A079, A082, A083, A086, A088, A091, |
| | A093, A095, A096, A097, A101, A103, A105, A107, A123, |
| | A127, A128, A129, A131, A132, A135, A141, A147, A148, |
| | A149, A150, A153, A154, A158, A164, A166, A178, A182, |
| | A189, A193, A194, A196, A206, A207, A237, A241, A244, |
| | A245, A246, A247, A250, A251, A252, A257, A261, A261, |
| | A263, A267, A269, A280, A281, A281, A296, A297, A305, |
| | A306, A328, A336, A337, A349, A350, A351, A353, A354, |
| | A357, A360, A361, A368, A369, A379, A384, A386, A387, |
| | A393, A394, A395, A397, A406, A407, A408, A409, A410, |
| | A415, A416, A417, A419, A420, A421, A430, A431, A432, |
| | A435, A436, A437, A438, A443, A455, A463, A470, A490, |
| | A502, A515, A517, A518, A519, A529, A536, A538, A539, |
| | A541, A543, A544, A545, A548, A554, A555, A557, A558, |
| | A560, A564, A570, A571, A572, A573, A575, A578, A580, |
| | A581, A582, A583, A586, A591, A594, A603, A604, A605, |
| | A606, A608, A611, A612, A618, A620, A622, A624, A625, |
| | A630, A631, A632, A633, A634, A635, A636, A650, A651, |
| | A653, A654, A655, A656, A659, A661, A662, A667, A668, |
| | A669, A670, A671, A672, A677, A678, A680, A681, A682, |
| | A683, A684, A685, A686, A687, A689, A690, A695, A696, |
| | A697, A698, A699, A700, A701, A702, A704, A709, A710, |
| | A711, A713, A714, A717, A718, A719, A728, A729, A730, |
| | A731, A732, A737 |
| ++++ | A001, A003, A006, A007, A008, A010, A013, A014, A017, |
| | A018, A019, A020, A025, A030, A031, A033, A035, A036, |
| | A038, A039, A040, A045, A050, A053, A060, A061, A062, |
| | A063, A066, A067, A068, A069, A071, A073, A075, A081, |
| | A081, A089, A092, A098, A099, A104, A108, A109, A110, |
| | A111, A112, A113, A115, A115, A117, A119, A120, A121, |
| | A124, A125, A131, A132, A133, A133, A134, A135, A137, |
| | A140, A143, A145, A146, A151, A155, A159, A161, A162, |
| | A167, A168, A169, A172, A173, A175, A176, A179, A180, |
| | A181, A182, A183, A184, A186, A187, A197, A199, A202, |
| | A204, A209, A210, A211, A212, A213, A214, A214, A215, |
| | A216, A217, A221, A223, A225, A225, A226, A227, A238, |
| | A241, A247, A249, A250, A251, A253, A254, A258, A258, |
| | A259, A277, A278, A282, A283, A290, A291, A301, A302, |
| | A309, A310, A314, A317, A320, A321, A324, A325, A326, |
| | A327, A330, A331, A332, A335, A339, A340, A341, A342, |
| | A343, A344, A347, A348, A356, A358, A359, A363, A364, |
| | A365, A366, A370, A371, A372, A373, A376, A378, A380, |
| | A381, A390, A391, A392, A396, A399, A401, A402, A411, |
| | A412, A413, A414, A422, A423, A424, A425, A426, A427, |
| | A439, A440, A442, A444, A445, A446, A447, A449, A451, |
| | A452, A454, A456, A457, A458, A459, A461, A462, A466, |
| | A468, A469, A471, A472, A473, A474, A475, A476, A481, |
| | A485, A487, A488, A489, A495, A498, A499, A501, A503, |
| | A504, A505, A506, A507, A509, A510, A511, A512, A513, |
| | A520, A521, A522, A525, A526, A528, A530, A531, A532, |
| | A533, A534, A535, A540, A546, A547, A549, A550, A551, |
| | A552, A553, A559, A561, A562, A563, A565, A566, A567, |
| | A568, A569, A574, A579, A585, A588, A590, A592, A593, |
| | A595, A597, A600, A601, A607, A610, A616, A619, A623, |
| | A626, A628, A637, A638, A639, A640, A641, A642, A645, |
| | A647, A652, A658, A660, A673, A676, A688, A691, A692, |
| | A693, A694, A705, A706, A707, A716, A720, A722, A723, |
| | A725, A726, A727, A733, A738, A739 |
| +++++ | A002, A004, A009, A011, A012, A015, A021, A023, A024, |
| | A026, A032, A036, A037, A038, A042, A046, A048, A049, |
| | A053, A059, A065, A078, A078, A080, A084, A085, A090, |
| | A094, A100, A102, A106, A114, A116, A118, A120, A121, |
| | A122, A124, A126, A130, A133, A136, A138, A139, A142, |
| | A144, A144, A148, A152, A156, A157, A160, A163, A165, |
| | A170, A171, A174, A177, A185, A186, A187, A188, A190, |
| | A191, A191, A192, A192, A195, A196, A198, A200, A201, |
| | A203, A205, A208, A209, A210, A210, A210, A211, A211, |
| | A211, A212, A213, A214, A214, A215, A216, A217, A218, |
| | A218, A219, A219, A219, A219, A220, A220, A221, A222, |
| | A222, A223, A224, A224, A225, A225, A228, A229, A230, |
| | A231, A231, A231, A232, A234, A235, A236, A239, A239, |
| | A240, A242, A243, A248, A271, A272, A273, A274, A276, |
| | A279, A279, A279, A283, A283, A284, A285, A286, A287, |
| | A288, A289, A293, A303, A304, A304, A304, A304, A307, |
| | A308, A311, A312, A313, A316, A316, A316, A318, A318, |
| | A322, A323, A328, A329, A345, A346, A352, A355, A362, |
| | A374, A375, A377, A398, A400, A403, A404, A405, A428, |
| | A429, A441, A448, A450, A453, A460, A467, A477, A478, |
| | A480, A482, A483, A484, A486, A494, A496, A497, A500, |
| | A508, A514, A516, A523, A524, A556, A576, A584, A587, |
| | A589, A599, A613, A614, A615, A617, A627, A643, A644, |
| | A646, A648, A649, A657, A663, A666, A674, A675, A703, |
| | A708, A715, A721, A724, A735 |

TABLE 7
KRAS G12D FRET data

| IC50* | Examples |
|---|---|
| + | none |
| ++ | A183, A264, A265, A268, A319, A388, A464, A465, A664 |
| +++ | A028, A054, A075, A076, A076, A087, A107, A112, A123, |
| | A131, A145, A153, A155, A167, A184, A189, A194, A233, |
| | A247, A255, A256, A257, A260, A261, A262, A263, A266, |
| | A269, A270, A275, A292, A294, A295, A298, A300, A333, |
| | A334, A334, A338, A349, A353, A354, A367, A368, A382, |
| | A383, A384, A385, A386, A387, A389, A394, A407, A409, |
| | A417, A418, A432, A433, A436, A479, A491, A492, A493, |
| | A527, A529, A537, A542, A555, A577, A578, A594, A596, |
| | A598, A602, A603, A605, A608, A609, A612, A621, A622, |
| | A629, A633, A650, A651, A653, A654, A662, A665, A667, |
| | A669, A671, A678, A679, A681, A682, A683, A685, A696, |
| | A697, A698, A700, A701, A704, A709, A710, A711, A712, |
| | A719, A734, A736, A737 |
| ++++ | A005, A007, A012, A015, A016, A022, A029, A034, A037, |
| | A037, A038, A038, A039, A043, A044, A046, A047, A048, |
| | A051, A052, A055, A056, A057, A058, A067, A070, A070, |
| | A071, A072, A074, A077, A079, A082, A086, A088, A090, |
| | A091, A093, A096, A097, A101, A103, A105, A108, A110, |
| | A116, A120, A120, A121, A121, A127, A128, A129, A130, |
| | A131, A132, A133, A133, A133, A134, A135, A138, A139, |
| | A140, A141, A142, A144, A144, A147, A148, A149, A150, |
| | A152, A154, A155, A156, A157, A162, A163, A164, A166, |
| | A175, A178, A179, A180, A181, A182, A183, A194, A196, |
| | A207, A213, A218, A218, A220, A224, A224, A237, A241, |
| | A244, A245, A246, A247, A250, A251, A252, A253, A254, |
| | A258, A261, A267, A280, A281, A281, A282, A296, A297, |
| | A299, A305, A306, A314, A326, A328, A330, A332, A336, |
| | A337, A341, A342, A350, A351, A356, A357, A358, A360, |
| | A361, A365, A366, A369, A370, A373, A379, A380, A381, |
| | A390, A393, A395, A396, A397, A406, A408, A410, A411, |
| | A412, A415, A416, A419, A420, A421, A422, A430, A431, |
| | A435, A437, A438, A440, A443, A444, A451, A454, A455, |
| | A456, A459, A463, A470, A476, A487, A488, A490, A499, |
| | A501, A502, A503, A504, A505, A510, A513, A515, A517, |
| | A518, A519, A520, A525, A534, A536, A538, A539, A541, |
| | A543, A544, A545, A546, A547, A548, A550, A551, A552, |
| | A553, A554, A557, A558, A559, A560, A561, A562, A564, |
| | A566, A567, A570, A571, A572, A573, A575, A580, A581, |
| | A582, A583, A586, A588, A591, A593, A595, A600, A604, |
| | A606, A607, A610, A611, A618, A619, A620, A623, A624, |
| | A625, A626, A630, A631, A632, A634, A635, A636, A640, |
| | A645, A647, A655, A656, A658, A659, A660, A661, A668, |
| | A670, A672, A673, A676, A677, A680, A684, A686, A687, |
| | A688, A689, A690, A691, A694, A695, A699, A702, A707, |
| | A713, A714, A717, A718, A722, A727, A728, A729, A730, |
| | A731, A732, A738, A739 |
| +++++ | A001, A002, A003, A004, A004, A006, A008, A009, A010, |
| | A011, A012, A013, A014, A015, A017, A018, A019, A020, |
| | A021, A023, A024, A025, A026, A027, A030, A031, A032, |
| | A032, A033, A035, A036, A036, A037, A040, A041, A042, |
| | A045, A048, A049, A050, A053, A053, A059, A060, A061, |
| | A062, A063, A064, A065, A066, A068, A069, A073, A075, |
| | A078, A078, A080, A081, A081, A083, A084, A085, A089, |
| | A092, A094, A095, A098, A099, A100, A102, A104, A106, |
| | A109, A111, A112, A113, A114, A115, A115, A117, A118, |
| | A119, A122, A124, A124, A125, A126, A132, A135, A136, |
| | A137, A143, A145, A146, A148, A151, A158, A159, A160, |
| | A161, A165, A167, A168, A169, A170, A171, A172, A173, |
| | A174, A176, A177, A181, A182, A184, A185, A186, A186, |
| | A187, A187, A188, A190, A191, A191, A192, A192, A193, |
| | A195, A196, A197, A198, A199, A200, A201, A202, A203, |
| | A204, A205, A206, A208, A209, A209, A210, A210, A210, |
| | A210, A211, A211, A211, A211, A212, A212, A213, A214, |
| | A214, A214, A214, A215, A215, A216, A216, A217, A217, |
| | A219, A219, A219, A219, A220, A221, A221, A222, A222, |
| | A223, A223, A225, A225, A225, A225, A226, A227, A228, |
| | A229, A230, A231, A231, A231, A232, A234, A235, A236, |
| | A238, A239, A239, A240, A241, A242, A243, A248, A249, |
| | A250, A251, A258, A259, A271, A272, A273, A274, A276, |
| | A277, A278, A279, A279, A279, A283, A283, A283, A284, |
| | A285, A286, A287, A288, A289, A290, A291, A293, A301, |
| | A302, A303, A304, A304, A304, A304, A307, A308, A309, |
| | A310, A311, A312, A313, A316, A316, A316, A317, A318, |
| | A318, A320, A321, A322, A323, A324, A325, A327, A328, |
| | A329, A331, A335, A339, A340, A343, A344, A345, A346, |
| | A347, A348, A352, A355, A359, A362, A363, A364, A371, |
| | A372, A374, A375, A376, A377, A378, A391, A392, A398, |
| | A399, A400, A401, A402, A403, A404, A405, A413, A414, |
| | A423, A424, A425, A426, A427, A428, A429, A439, A441, |
| | A442, A445, A446, A447, A448, A449, A450, A452, A453, |
| | A457, A458, A460, A461, A462, A466, A467, A468, A469, |
| | A471, A472, A473, A474, A475, A477, A478, A480, A481, |
| | A482, A483, A484, A485, A486, A489, A494, A495, A496, |
| | A497, A498, A500, A506, A507, A508, A509, A511, A512, |
| | A514, A516, A521, A522, A523, A524, A526, A528, A530, |
| | A531, A532, A533, A535, A540, A549, A556, A563, A565, |
| | A568, A569, A574, A576, A579, A584, A585, A587, A589, |
| | A590, A592, A597, A599, A601, A613, A614, A615, A616, |
| | A617, A627, A628, A637, A638, A639, A641, A642, A643, |
| | A644, A646, A648, A649, A652, A657, A663, A666, A674, |
| | A675, A692, A693, A703, A705, A706, A708, A715, A716, |
| | A720, A721, A723, A724, A725, A726, A733, A735 |

TABLE 8
KRAS G13C FRET data

| IC50* | Examples |
|---|---|
| + | A088, A096, A097, A107, A131, A132, A155, A175, A247, |
| | A250, A251, A252, A253, A255, A258, A262, A270, A292, |
| | A294, A297, A298, A299, A334, A336, A338, A474, A475, |
| | A529, A542, A683 |
| ++ | A031, A037, A038, A046, A067, A069, A071, A075, A076, |
| | A076, A090, A108, A112, A116, A121, A123, A124, A131, |
| | A132, A133, A133, A135, A140, A141, A145, A150, A155, |
| | A163, A167, A172, A178, A182, A183, A194, A196, A218, |
| | A224, A233, A244, A245, A246, A247, A249, A250, A251, |
| | A254, A256, A257, A258, A259, A260, A261, A263, A264, |
| | A265, A266, A267, A268, A275, A280, A281, A283, A295, |
| | A296, A300, A305, A306, A319, A333, A334, A337, A379, |
| | A383, A385, A388, A389, A394, A396, A397, A417, A418, |
| | A421, A432, A433, A438, A452, A464, A465, A479, A491, |
| | A492, A493, A517, A525, A527, A537, A538, A539, A540, |
| | A541, A545, A546, A552, A553, A554, A555, A564, A570, |
| | A571, A572, A573, A574, A575, A577, A578, A591, A594, |
| | A595, A597, A600, A602, A603, A604, A605, A606, A607, |
| | A608, A609, A610, A621, A622, A629, A630, A632, A650, |
| | A664, A668, A679, A680, A695, A698, A699, A706, A711, |
| | A714, A718, A719, A734, A736, A737 |
| +++ | A005, A012, A013, A016, A018, A022, A027, A028, A029, |
| | A033, A036, A037, A038, A043, A044, A047, A048, A049, |
| | A052, A054, A055, A056, A057, A070, A070, A072, A074, |
| | A077, A082, A086, A087, A091, A093, A095, A101, A105, |
| | A110, A117, A120, A125, A126, A127, A128, A129, A130, |
| | A135, A138, A142, A146, A147, A149, A153, A157, A158, |
| | A162, A164, A165, A166, A179, A180, A181, A184, A189, |
| | A196, A199, A207, A209, A213, A216, A218, A220, A220, |
| | A222, A224, A232, A237, A238, A241, A241, A261, A269, |
| | A277, A281, A302, A314, A322, A323, A324, A328, A332, |
| | A340, A341, A342, A344, A349, A350, A351, A353, A354, |
| | A356, A357, A358, A360, A361, A365, A366, A367, A368, |
| | A369, A370, A371, A372, A373, A380, A382, A384, A386, |
| | A387, A390, A393, A395, A401, A406, A407, A408, A409, |
| | A410, A411, A412, A415, A416, A419, A420, A422, A423, |
| | A425, A430, A431, A435, A436, A437, A439, A440, A443, |
| | A444, A445, A449, A451, A454, A455, A457, A458, A459, |
| | A463, A466, A470, A471, A476, A485, A487, A488, A490, |
| | A497, A501, A502, A503, A504, A505, A509, A510, A513, |
| | A515, A518, A519, A524, A534, A536, A543, A544, A547, |
| | A549, A550, A551, A557, A558, A559, A560, A561, A562, |
| | A563, A566, A567, A576, A580, A581, A582, A583, A586, |
| | A593, A596, A598, A601, A611, A612, A618, A619, A620, |
| | A624, A625, A631, A633, A634, A635, A636, A640, A642, |
| | A645, A647, A651, A652, A653, A654, A655, A658, A659, |
| | A660, A661, A662, A665, A667, A669, A670, A671, A672, |
| | A673, A678, A681, A682, A684, A685, A686, A687, A688, |
| | A689, A690, A692, A694, A696, A697, A700, A701, A702, |
| | A704, A707, A709, A710, A712, A717, A722, A725, A726, |
| | A727, A728, A729, A730, A731, A732, A733, A738, A739 |
| ++++ | A001, A003, A004, A006, A007, A008, A010, A014, A015, |
| | A017, A019, A020, A025, A030, A032, A034, A035, A039, |
| | A040, A041, A045, A051, A058, A060, A061, A063, A064, |
| | A068, A073, A075, A078, A078, A079, A081, A081, A083, |
| | A089, A092, A098, A099, A103, A104, A111, A112, A115, |
| | A115, A119, A120, A121, A124, A133, A134, A139, A144, |
| | A144, A145, A148, A152, A154, A156, A161, A167, A168, |
| | A169, A170, A171, A174, A176, A181, A182, A183, A186, |
| | A187, A191, A193, A194, A197, A200, A201, A204, A206, |
| | A212, A213, A214, A214, A214, A217, A221, A225, A225, |
| | A225, A227, A235, A242, A278, A279, A282, A283, A283, |
| | A284, A289, A290, A291, A301, A307, A308, A309, A310, |
| | A316, A316, A317, A320, A321, A325, A326, A327, A330, |
| | A331, A335, A339, A343, A345, A346, A347, A348, A359, |
| | A362, A363, A364, A375, A376, A377, A378, A381, A391, |
| | A392, A398, A399, A400, A402, A403, A413, A414, A424, |
| | A426, A427, A441, A442, A446, A447, A456, A460, A461, |
| | A462, A467, A468, A469, A472, A473, A477, A480, A481, |
| | A483, A484, A486, A489, A494, A495, A496, A498, A499, |
| | A506, A507, A508, A511, A512, A514, A516, A520, A521, |
| | A522, A526, A528, A530, A531, A532, A533, A535, A548, |
| | A556, A565, A568, A569, A579, A584, A585, A588, A589, |
| | A590, A592, A599, A614, A616, A623, A626, A628, A637, |
| | A638, A639, A641, A643, A644, A648, A656, A657, A663, |
| | A674, A676, A677, A691, A693, A703, A705, A708, A713, |
| | A716, A720, A721, A723, A735 |
| +++++ | A210, A173, A109, A042, A062, A137, A303, A228, A666, |
| | A214, A225, A627, A675, A229, A015, A066, A184, A209, |
| | A328, A288, A011, A587, A448, A613, A617, A226, A240, |
| | A316, A113, A059, A002, A374, A223, A313, A190, A212, |
| | A219, A065, A032, A615, A143, A215, A080, A026, A050, |
| | A191, A219, A216, A217, A106, A649, A151, A053, A478, |
| | A222, A219, A037, A211, A404, A724, A085, A248, A202, |
| | A122, A159, A094, A004, A009, A012, A021, A023, A024, |
| | A036, A048, A053, A084, A100, A102, A114, A118, A136, |
| | A148, A160, A177, A185, A186, A187, A188, A192, A192, |
| | A195, A198, A203, A205, A208, A210, A210, A210, A211, |
| | A211, A211, A215, A219, A221, A223, A230, A231, A231, |
| | A231, A234, A236, A239, A239, A243, A271, A272, A273, |
| | A274, A276, A279, A279, A285, A286, A287, A293, A304, |
| | A304, A304, A304, A311, A312, A318, A318, A329, A352, |
| | A355, A405, A428, A429, A450, A453, A482, A500, A523, |
| | A646, A715 |

TABLE 9
KRAS G12V FRET data

| IC50* | Examples |
|---|---|
| + | None |
| ++ | A028, A075, A076, A076, A087, A112, A132, A145, A155, |
| | A167, A183, A184, A194, A233, A245, A262, A264, A265, |
| | A266, A268, A275, A292, A295, A298, A300, A305, A319, |
| | A333, A334, A338, A368, A383, A388, A389, A418, A464, |
| | A465, A479, A491, A492, A493, A527, A537, A542, A602, |
| | A609, A621, A629, A665, A679, A734 |
| +++ | A004, A005, A012, A015, A016, A022, A027, A029, A034, |
| | A037, A037, A041, A043, A044, A047, A048, A051, A052, |
| | A054, A055, A056, A057, A058, A069, A070, A072, A074, |
| | A077, A079, A082, A086, A088, A091, A093, A095, A096, |
| | A097, A101, A103, A105, A107, A123, A127, A128, A129, |
| | A131, A135, A141, A147, A148, A149, A150, A153, A154, |
| | A155, A162, A164, A166, A178, A180, A181, A182, A183, |
| | A189, A193, A194, A196, A206, A207, A209, A237, A238, |
| | A241, A244, A246, A247, A250, A251, A252, A253, A255, |
| | A256, A257, A258, A260, A261, A263, A267, A269, A270, |
| | A280, A281, A281, A294, A296, A297, A299, A306, A334, |
| | A336, A337, A349, A350, A353, A354, A357, A360, A361, |
| | A366, A367, A369, A373, A379, A380, A382, A384, A385, |
| | A386, A387, A394, A395, A397, A406, A407, A408, A409, |
| | A410, A415, A416, A417, A419, A420, A421, A431, A432, |
| | A433, A435, A436, A437, A438, A443, A444, A463, A490, |
| | A517, A518, A519, A529, A536, A538, A539, A543, A544, |
| | A545, A546, A548, A550, A554, A555, A564, A570, A571, |
| | A572, A577, A578, A580, A581, A582, A586, A591, A594, |
| | A596, A598, A603, A604, A605, A606, A608, A611, A612, |
| | A618, A620, A622, A624, A625, A630, A631, A632, A633, |
| | A634, A635, A650, A651, A653, A654, A655, A659, A660, |
| | A662, A664, A667, A668, A669, A670, A671, A672, A673, |
| | A678, A680, A681, A682, A683, A684, A685, A686, A687, |
| | A695, A696, A697, A698, A699, A700, A701, A702, A704, |
| | A709, A710, A711, A712, A714, A717, A718, A719, A728, |
| | A736, A737 |
| ++++ | A001, A003, A006, A007, A008, A010, A013, A017, A018, |
| | A025, A030, A031, A032, A035, A036, A038, A039, A040, |
| | A045, A046, A050, A060, A061, A062, A063, A064, A066, |
| | A067, A068, A070, A071, A073, A075, A080, A081, A081, |
| | A083, A089, A092, A098, A099, A104, A108, A109, A110, |
| | A111, A112, A113, A115, A115, A117, A120, A121, A124, |
| | A125, A131, A132, A133, A133, A134, A135, A137, A138, |
| | A140, A143, A145, A146, A151, A152, A158, A159, A161, |
| | A167, A168, A169, A172, A173, A175, A176, A179, A181, |
| | A182, A184, A186, A187, A191, A197, A199, A204, A210, |
| | A211, A212, A213, A215, A216, A217, A218, A221, A223, |
| | A224, A226, A227, A232, A241, A242, A247, A249, A251, |
| | A254, A258, A259, A261, A277, A278, A282, A283, A289, |
| | A290, A291, A301, A302, A307, A309, A310, A314, A316, |
| | A317, A320, A321, A324, A325, A326, A328, A330, A331, |
| | A332, A335, A339, A340, A341, A342, A343, A344, A346, |
| | A348, A351, A356, A358, A359, A362, A363, A364, A365, |
| | A370, A372, A375, A376, A378, A381, A390, A391, A392, |
| | A393, A396, A398, A399, A401, A402, A403, A411, A412, |
| | A413, A414, A422, A423, A424, A426, A427, A430, A439, |
| | A440, A445, A446, A447, A449, A451, A452, A454, A455, |
| | A456, A457, A458, A459, A461, A462, A466, A468, A469, |
| | A470, A472, A473, A474, A475, A476, A477, A481, A483, |
| | A485, A487, A488, A489, A495, A498, A499, A501, A502, |
| | A503, A504, A505, A506, A507, A509, A510, A511, A512, |
| | A513, A515, A520, A521, A522, A525, A526, A528, A530, |
| | A531, A532, A533, A534, A540, A541, A547, A549, A551, |
| | A552, A553, A556, A557, A558, A559, A560, A561, A562, |
| | A563, A565, A566, A567, A573, A574, A575, A579, A583, |
| | A584, A585, A588, A590, A592, A593, A595, A597, A599, |
| | A600, A601, A607, A610, A616, A619, A623, A626, A628, |
| | A636, A637, A638, A639, A640, A641, A642, A645, A647, |
| | A652, A656, A658, A661, A676, A677, A688, A689, A690, |
| | A691, A692, A693, A694, A703, A705, A706, A707, A708, |
| | A713, A716, A720, A721, A722, A723, A725, A726, A727, |
| | A729, A730, A731, A732, A733, A738, A739 |
| +++++ | A002, A004, A009, A011, A012, A014, A015, A019, A020, |
| | A021,A023, A024, A026, A032, A033, A036, A037, A038, |
| | A042, A048, A049, A053, A053, A059, A065, A078, A078, |
| | A084, A085, A090, A094, A100, A102, A106, A114, A116, |
| | A118, A119, A120, A121, A122, A124, A126, A130, A133, |
| | A136, A139, A142, A144, A144, A148, A156, A157, A160, |
| | A163, A165, A170, A171, A174, A177, A185, A186, A187, |
| | A188, A190, A191, A192, A192, A195, A196, A198, A200, |
| | A201, A202, A203, A205, A208, A209, A210, A210, A210, |
| | A211, A211, A211, A212, A213, A214, A214, A214, A214, |
| | A215, A216, A217, A218, A219, A219, A219, A219, A220, |
| | A220,A221, A222, A222, A223, A224, A225, A225, A225, |
| | A225, A228, A229, A230, A231, A231, A231, A234, A235, |
| | A236, A239, A239, A240, A243, A248, A250, A271, A272, |
| | A273, A274, A276, A279, A279, A279, A283, A283, A284, |
| | A285, A286, A287, A288, A293, A303, A304, A304, A304, |
| | A304, A308, A311, A312, A313, A316, A316, A318, A318, |
| | A322, A323, A327, A328, A329, A345, A347, A352, A355, |
| | A371, A374, A377, A400, A404, A405, A425, A428, A429, |
| | A441, A442, A448, A450, A453, A460, A467, A471, A478, |
| | A480, A482, A484, A486, A494, A496, A497, A500, A508, |
| | A514, A516, A523, A524, A535, A568, A569, A576, A587, |
| | A589, A613, A614, A615, A617, A627, A643, A644, A646, |
| | A648, A649, A657, A663, A666, A674, A675, A715, A724, |
| | A735 |

TABLE 10
KRAS WT FRET data

| IC50* | Examples |
|---|---|
| + | A388 |
| ++ | A075, A076, A076, A112, A132, A155, A167, A183, A194, |
| | A233, A256, A260, A261, A262, A263, A264, A265, A268, |
| | A270, A280, A294, A295, A296, A297, A298, A299, A300, |
| | A319, A333, A334, A338, A367, A382, A383, A385, A386, |
| | A387, A389, A418, A432, A433, A436, A464, A465, A479, |
| | A491, A492, A493, A527, A537, A542, A569, A594, A598, |
| | A602, A603, A605, A609, A621, A629, A633, A664, A665, |
| | A667, A679, A704, A711, A712, A734, A736, A737 |
| +++ | A005, A012, A016, A022, A028, A029, A037, A037, A043, |
| | A052, A054, A056, A069, A070, A070, A072, A074, A077, |
| | A082, A087, A088, A091, A093, A096, A097, A101, A105, |
| | A107, A110, A123, A128, A131, A141, A145, A147, A148, |
| | A149, A150, A153, A158, A164, A166, A180, A181, A182, |
| | A184, A189, A207, A244, A245, A246, A247, A250, A251, |
| | A252, A253, A254, A255, A257, A258, A259, A266, A267, |
| | A269, A275, A281, A281, A292, A305, A306, A334, A336, |
| | A337, A349, A350, A351, A353, A354, A356, A357, A358, |
| | A360, A361, A368, A369, A379, A384, A393, A394, A395, |
| | A397, A406, A407, A408, A409, A410, A415, A416, A417, |
| | A419, A420, A421, A430, A431, A435, A437, A438, A452, |
| | A454, A455, A457, A463, A490, A501, A517, A518, A519, |
| | A529, A536, A538, A539, A541, A543, A544, A545, A547, |
| | A548, A550, A552, A553, A554, A555, A557, A558, A564, |
| | A570, A571, A572, A573, A575, A577, A578, A580, A581, |
| | A582, A583, A586, A591, A595, A600, A604, A606, A607, |
| | A608, A610, A611, A612, A618, A620, A622, A624, A625, |
| | A630, A631, A632, A634, A635, A636, A647, A650, A651, |
| | A653, A654, A655, A656, A659, A660, A661, A662, A668, |
| | A669, A670, A671, A672, A676, A677, A678, A680, A681, |
| | A682, A683, A684, A685, A686, A687, A688, A690, A695, |
| | A696, A697, A698, A699, A700, A701, A702, A709, A710, |
| | A713, A714, A717, A718, A719, A722, A728, A729, A730, |
| | A731, A732, A738, A739 |
| ++++ | A004, A006, A007, A008, A010, A013, A014, A015, A017, |
| | A018,A019, A020, A025, A027, A030, A031, A032, A033, |
| | A034, A035, A036, A038, A039, A040, A041, A044, A045, |
| | A047, A048, A051, A055, A057, A058, A061, A063, A064, |
| | A067, A068, A071, A073, A075, A079, A081, A081, A083, |
| | A086, A089, A092, A095, A098, A103, A104, A108, A111, |
| | A112, A115, A115, A116, A117, A120, A121, A124, A125, |
| | A127, A129, A131, A132, A133, A133, A134, A135, A137, |
| | A138, A140, A145, A146, A152, A154, A155, A159, A161, |
| | A162, A163, A167, A168, A169, A172, A175, A178, A179, |
| | A181, A182, A183, A187, A193, A194, A196, A197, A199, |
| | A204, A206, A209, A212, A213, A214, A214, A214, A214, |
| | A216, A217, A222, A225, A225, A225, A225, A227, A232, |
| | A237, A238, A241, A241, A247, A249, A250, A251, A258, |
| | A261, A277, A278, A282, A283, A290, A291, A302, A309, |
| | A310, A314, A317, A320, A321, A324, A326, A328, A330, |
| | A331, A332, A335, A339, A340, A341, A342, A343, A344, |
| | A347, A348, A352, A359, A363, A364, A365, A366, A370, |
| | A371, A372, A373, A375, A376, A378, A380, A381, A390, |
| | A391, A392, A396, A398, A399, A401, A402, A411, A412, |
| | A413, A422, A423, A424, A426, A427, A439, A440, A443, |
| | A444, A445, A446, A447, A449, A451, A456, A458, A459, |
| | A461, A462, A466, A468, A469, A470, A471, A472, A473, |
| | A474, A475, A476, A477, A481, A483, A485, A487, A488, |
| | A489, A494, A495, A497, A498, A499, A502, A503, A504, |
| | A505, A506, A509, A510, A511, A512, A513, A515, A520, |
| | A521, A522, A525, A526, A528, A530, A531, A532, A533, |
| | A534, A540, A546, A549, A551, A556, A559, A560, A561, |
| | A562, A563, A566, A567, A574, A576, A579, A585, A588, |
| | A589, A590, A593, A597, A601, A616, A619, A623, A626, |
| | A637, A638, A639, A640, A641, A642, A645, A648, A652, |
| | A658, A673, A689, A691, A692, A693, A694, A703, A705, |
| | A706, A707, A708, A716, A720, A723, A725, A726, A727, |
| | A735 |
| +++++ | A001, A002, A003, A004, A009, A011, A012, A015, A021, |
| | A023, A024, A026, A032, A036, A037, A038, A042, A046, |
| | A048, A049, A050, A053, A053, A059, A060, A062, A065, |
| | A066, A078, A078, A080, A084, A085, A090, A094, A099, |
| | A100, A102, A106, A109, A113, A114, A118, A119, A120, |
| | A121, A122, A124, A126, A130, A133, A135, A136, A139, |
| | A142, A143, A144, A144, A148, A151, A156, A157, A160, |
| | A165, A170, A171, A173, A174, A176, A177, A184, A185, |
| | A186, A186, A187, A188, A190, A191, A191, A192, A192, |
| | A195, A196, A198, A200, A201, A202, A203, A205, A208, |
| | A209, A210, A210, A210, A210, A211, A211, A211, A211, |
| | A212, A213, A215, A215, A216, A217, A218, A218, A219, |
| | A219, A219, A219, A220, A220, A221, A221, A222, A223, |
| | A223, A224, A224, A226, A228, A229, A230, A231, A231, |
| | A231, A234, A235, A236, A239, A239, A240, A242, A243, |
| | A248, A271, A272, A273, A274, A276, A279, A279, A279, |
| | A283, A283, A284, A285, A286, A287, A288, A289, A293, |
| | A301, A303, A304, A304, A304, A304, A307, A308, A311, |
| | A312, A313, A316, A316, A316, A318, A318, A322, A323, |
| | A325, A327, A328, A329, A345, A346, A355, A362, A374, |
| | A377, A400, A403, A404, A405, A414, A425, A428, A429, |
| | A441, A442, A448, A450, A453, A460, A467, A478, A480, |
| | A482, A484, A486, A496, A500, A507, A508, A514, A516, |
| | A523, A524, A535, A565, A568, A584, A587, A592, A596, |
| | A599, A613, A614, A615, A617, A627, A628, A643, A644, |
| | A646, A649, A657, A663, A666, A674, A675, A715, A721, |
| | A724, A733 |

TABLE 11
KRAS G12C FRET data

| IC50* | Examples |
|---|---|
| + | A038, A075, A076, A088, A095, A096, A097, A107, A108, |
| | A112, A116, A120, A121, A129, A130, A131, A131, A132, |
| | A133, A133, A135, A140, A141, A145, A157, A162, A167, |
| | A175, A176, A182, A183, A184, A194, A196, A209, A244, |
| | A245, A246, A247, A247, A255, A256, A257, A266, A268, |
| | A270, A290, A291, A292, A294, A298, A299, A300, A316, |
| | A316, A316, A317, A322, A323, A324, A326, A333, A334, |
| | A337, A339, A343, A346, A348, A351, A365, A375, A376, |
| | A377, A379, A380, A381, A388, A391, A403, A407, A411, |
| | A414, A425, A427, A474, A475, A477, A479, A509, A527, |
| | A529, A534, A542, A543, A544, A549, A550, A551, A556, |
| | A577, A578, A579, A591, A599, A600, A601, A609, A620, |
| | A626, A627, A628, A638, A642, A647, A658, A660, A663, |
| | A667, A673, A684, A689, A691, A692, A699, A705, A707, |
| | A711, A721, A726, A727, A735 |
| ++ | A004, A005, A012, A015, A016, A020, A022, A027, A028, |
| | A029, A031, A032, A037, A037, A038, A043, A044, A047, |
| | A048, A051, A052, A054, A055, A056, A057, A058, A064, |
| | A067, A069, A070, A070, A071, A072, A074, A076, A077, |
| | A079, A082, A083, A086, A087, A091, A093, A103, A104, |
| | A105, A115, A121, A123, A124, A126, A127, A128, A132, |
| | A133, A134, A135, A138, A139, A142, A144, A148, A149, |
| | A150, A152, A153, A154, A155, A155, A156, A158, A164, |
| | A165, A166, A172, A178, A179, A181, A182, A183, A186, |
| | A187, A189, A191, A193, A196, A200, A201, A204, A207, |
| | A209, A214, A214, A225, A225, A233, A237, A238, A241, |
| | A241, A250, A250, A251, A251, A252, A253, A260, A261, |
| | A262, A263, A264, A265, A267, A269, A275, A280, A281, |
| | A283, A295, A296, A297, A305, A319, A328, A332, A334, |
| | A336, A338, A342, A344, A349, A350, A352, A353, A359, |
| | A361, A362, A363, A366, A367, A368, A370, A371, A382, |
| | A383, A384, A385, A386, A387, A389, A394, A396, A397, |
| | A400, A410, A416, A417, A418, A421, A423, A432, A433, |
| | A435, A436, A437, A438, A439, A440, A443, A444, A445, |
| | A448, A452, A454, A456, A458, A459, A463, A464, A465, |
| | A466, A476, A478, A485, A489, A490, A491, A492, A493, |
| | A499, A501, A503, A504, A505, A510, A513, A515, A517, |
| | A518, A519, A525, A526, A528, A532, A533, A536, A537, |
| | A538, A545, A552, A558, A559, A560, A566, A567, A568, |
| | A569, A572, A573, A575, A580, A581, A584, A585, A587, |
| | A589, A590, A592, A594, A595, A596, A597, A598, A602, |
| | A603, A610, A612, A614, A615, A616, A617, A621, A622, |
| | A624, A629, A632, A634, A636, A637, A643, A644, A645, |
| | A646, A649, A650, A651, A653, A657, A659, A661, A662, |
| | A664, A665, A668, A669, A671, A672, A674, A675, A678, |
| | A679, A681, A682, A683, A685, A686, A697, A698, A700, |
| | A701, A703, A704, A706, A709, A710, A712, A715, A719, |
| | A720, A722, A724, A730, A733, A734, A736, A737 |
| +++ | A001, A003, A006, A007, A008, A010, A011, A013, A014, |
| | A017, A018, A019, A023, A025, A026, A030, A033, A034, |
| | A035, A039, A040, A041, A042, A045, A046, A050, A053, |
| | A053, A060, A061, A062, A063, A065, A066, A068, A073, |
| | A075, A078, A078, A080, A081, A081, A089, A092, A094, |
| | A098, A099, A101, A109, A110, A111, A112, A113, A115, |
| | A117, A118, A119, A120, A125, A137, A143, A144, A145, |
| | A146, A147, A151, A159, A160, A161, A167, A168, A169, |
| | A173, A174, A177, A180, A181, A184, A185, A188, A190, |
| | A191, A192, A194, A195, A197, A199, A205, A206, A208, |
| | A210, A211, A212, A213, A215, A216, A217, A218, A220, |
| | A221, A222, A223, A224, A226, A227, A229, A232, A249, |
| | A254, A258, A258, A259, A261, A277, A278, A281, A282, |
| | A284, A301, A302, A306, A310, A314, A320, A321, A327, |
| | A330, A331, A335, A340, A341, A347, A354, A356, A357, |
| | A358, A360, A364, A369, A372, A373, A374, A378, A390, |
| | A392, A393, A395, A399, A402, A404, A406, A408, A409, |
| | A412, A413, A415, A419, A420, A422, A424, A426, A430, |
| | A431, A442, A446, A447, A449, A451, A455, A457, A461, |
| | A462, A468, A469, A470, A471, A472, A473, A481, A483, |
| | A484, A486, A487, A488, A494, A495, A497, A498, A502, |
| | A506, A507, A508, A511, A512, A520, A522, A524, A530, |
| | A531, A539, A540, A541, A546, A547, A548, A553, A554, |
| | A555, A557, A561, A562, A563, A564, A565, A570, A571, |
| | A574, A576, A582, A583, A586, A588, A593, A604, A605, |
| | A606, A607, A608, A611, A613, A618, A619, A623, A625, |
| | A630, A631, A633, A635, A639, A640, A641, A648, A652, |
| | A654, A655, A656, A670, A676, A677, A680, A687, A688, |
| | A690, A693, A694, A695, A696, A702, A708, A713, A714, |
| | A716, A717, A718, A723, A725, A728, A729, A731, A732, |
| | A738, A739 |
| ++++ | A002, A004, A009, A012, A015, A021, A024, A032, A036, |
| | A037, A048, A049, A059, A084, A085, A090, A100, A102, |
| | A106, A124, A136, A148, A163, A170, A171, A186, A187, |
| | A192, A202, A203, A210, A211, A212, A214, A214, A216, |
| | A218, A219, A219, A220, A222, A224, A225, A225, A228, |
| | A235, A240, A242, A248, A272, A274, A276, A279, A283, |
| | A283, A289, A303, A307, A309, A313, A325, A328, A345, |
| | A398, A401, A405, A441, A453, A460, A467, A480, A496, |
| | A514, A516, A521, A523, A535, A666 |
| +++++ | A036, A114, A122, A198, A210, A210, A211, A211, A213, |
| | A215, A217, A219, A219, A221, A223, A230, A231, A231, |
| | A231, A234, A236, A239, A239, A243, A271, A273, A279, |
| | A279, A285, A286, A287, A288, A293, A304, A304, A304, |
| | A304, A308, A311, A312, A318, A318, A329, A355, A428, |
| | A429, A450, A482, A500 |

TABLE 12
KRAS G13D FRET data

| IC50* | Examples |
|---|---|
| + | None |
| ++ | A075, A076, A112, A155, A183, A260, A261, A262, A263, |
| | A264, A265, A267, A268, A270, A294, A296, A298, A300, |
| | A319, A333, A338, A388, A464, A465, A527, A537, A542, |
| | A664 |
| +++ | A028, A054, A096, A105, A107, A123, A128, A131, A132, |
| | A141, A145, A149, A150, A153, A158, A164, A167, A181, |
| | A182, A184, A189, A194, A196, A207, A233, A244, A245, |
| | A246, A247, A250, A251, A252, A253, A255, A256, A257, |
| | A258, A261, A266, A269, A275, A280, A281, A281, A292, |
| | A295, A297, A299, A305, A306, A334, A334, A336, A337, |
| | A349, A350, A353, A354, A357, A361, A367, A368, A379, |
| | A382, A383, A384, A385, A386, A387, A389, A394, A395, |
| | A397, A406, A407, A410, A415, A416, A417, A418, A421, |
| | A432, A433, A436, A438, A463, A479, A490, A491, A492, |
| | A493, A518, A519, A529, A536, A538, A543, A544, A545, |
| | A555, A558, A573, A577, A578, A580, A581, A582, A591, |
| | A594, A596, A598, A602, A603, A605, A606, A608, A609, |
| | A611, A612, A620, A621, A622, A624, A629, A631, A633, |
| | A650, A651, A653, A654, A655, A659, A661, A662, A665, |
| | A667, A669, A670, A671, A672, A678, A679, A681, A682, |
| | A683, A684, A685, A686, A687, A690, A695, A697, A698, |
| | A699, A700, A701, A704, A709, A710, A711, A712, A718, |
| | A719, A729, A730, A734, A736, A737 |
| ++++ | A016, A038, A069, A075, A088, A095, A103, A104, A108, |
| | A110, A111, A112, A116, A117, A120, A120, A121, A121, |
| | A125, A127, A129, A130, A131, A132, A133, A133, A133, |
| | A134, A135, A138, A140, A142, A144, A144, A145, A146, |
| | A147, A148, A152, A154, A155, A156, A157, A159, A161, |
| | A162, A163, A166, A175, A178, A179, A180, A181, A182, |
| | A183, A187, A193, A194, A197, A206, A209, A212, A213, |
| | A232, A237, A238, A241, A241, A247, A249, A251, A254, |
| | A258, A259, A277, A278, A282, A290, A302, A314, A321, |
| | A326, A328, A330, A331, A332, A335, A339, A340, A341, |
| | A342, A344, A351, A356, A358, A359, A360, A363, A364, |
| | A365, A366, A369, A370, A372, A373, A380, A381, A390, |
| | A391, A393, A396, A399, A401, A408, A409, A411, A412, |
| | A413, A419, A420, A422, A424, A427, A430, A431, A435, |
| | A437, A439, A440, A443, A444, A445, A446, A449, A451, |
| | A452, A454, A455, A456, A457, A458, A459, A466, A468, |
| | A469, A470, A472, A473, A474, A475, A476, A485, A487, |
| | A488, A489, A499, A501, A502, A503, A504, A505, A509, |
| | A510, A511, A513, A515, A517, A520, A521, A522, A525, |
| | A534, A539, A540, A541, A546, A547, A548, A549, A550, |
| | A551, A552, A553, A554, A557, A559, A560, A561, A562, |
| | A563, A564, A566, A567, A570, A571, A572, A574, A575, |
| | A583, A586, A588, A593, A595, A597, A600, A601, A604, |
| | A607, A610, A616, A618, A619, A623, A625, A626, A630, |
| | A632, A634, A635, A636, A637, A638, A640, A641, A642, |
| | AA645, 647, A652, A656, A658, A660, A668, A673, A676, |
| | A677, A680, A688, A689, A691, A693, A694, A696, A702, |
| | A706, A707, A713, A714, A717, A720, A722, A725, A726, |
| | A727, A728, A731, A732, A738, A739 |
| +++++ | A024, A065, A094, A102, A106, A109, A113, A114, A115, |
| | A115, A118, A119, A122, A124, A124, A126, A135, A136, |
| | A137, A139, A143, A148, A151, A160, A165, A167, A168, |
| | A169, A170, A171, A172, A173, A174, A176, A177, A184, |
| | A185, A186, A186, A187, A188, A190, A191, A191, A192, |
| | A192, A195, A196, A198, A199, A200, A201, A202, A203, |
| | A204, A205, A208, A209, A210, A210, A210, A210, A211, |
| | A211, A211, A211, A212, A213, A214, A214, A214, A225, |
| | A225, A225, A226, A227, A228, A229, A230, A231, A231, |
| | A231, A234, A235, A236, A239, A239, A240, A242, A243, |
| | A248, A250, A271, A272, A273, A274, A276, A279, A279, |
| | A279, A283, A283, A283, A284, A285, A286, A287, A288, |
| | A289, A291, A293, A301, A303, A304, A304, A304, A304, |
| | A307, A308, A309, A310, A311, A312, A313, A316, A316, |
| | A316, A317, A318, A318, A320, A322, A323, A324, A325, |
| | A327, A328, A329, A343, A345, A346, A347, A348, A352, |
| | A355, A362, A371, A374, A375, A376, A377, A378, A392, |
| | A398, A400, A402, A403, A404, A405, A414, A423, A425, |
| | A426, A428, A429, A441, A442, A447, A448, A450, A453, |
| | A460, A461, A462, A467, A471, A477, A478, A480, A481, |
| | A482, A483, A484, A486, A494, A495, A496, A497, A498, |
| | A500, A506, A507, A508, A512, A514, A516, A523, A524, |
| | A526, A528, A530, A531, A532, A533, A535, A556, A565, |
| | A568, A569, A576, A579, A584, A585, A587, A589, A590, |
| | A592, A599, A613, A614, A615, A617, A627, A628, A639, |
| | A643, A644, A646, A648, A649, A657, A663, A666, A674, |
| | A675, A692, A703, A705, A708, A715, A716, A721, A723, |
| | A724, A733, A735 |

TABLE 13
KRAS Q61H FRET data

| IC50* | Examples |
|---|---|
| + | A297, A299, A388, A598, A621 |
| ++ | A012, A022, A075, A076, A077, A082, A087, A105, A148, |
| | A167, A181, A207, A262, A275, A281, A300, A333, A338, |
| | A349, A350, A353, A354, A367, A368, A379, A382, A383, |
| | A384, A385, A386, A387, A389, A394, A407, A416, A417, |
| | A463, A491, A492, A493, A537, A542, A543, A544, A545, |
| | A557, A577, A578, A580, A582, A586, A594, A596, A602, |
| | A603, A605, A606, A608, A609, A611, A612, A620, A622, |
| | A629, A631, A633, A650, A651, A653, A654, A655, A656, |
| | A661, A662, A664, A665, A667, A668, A669, A671, A672, |
| | A676, A678, A679, A681, A682, A683, A684, A686, A687, |
| | A696, A697, A698, A701, A702, A704, A709, A710, A711, |
| | A712, A718, A719, A734, A736, A737, A739 |
| +++ | A016, A045, A064, A074, A083, A086, A089, A104, A115, |
| | A166, A172, A193, A252, A277, A314, A328, A330, A332, |
| | A340, A341, A342, A344, A351, A356, A357, A358, A360, |
| | A361, A365, A366, A369, A372, A373, A390, A393, A468, |
| | A474, A475, A494, A502, A503, A504, A505, A522, A534, |
| | A538, A539, A540, A541, A546, A547, A548, A549, A551, |
| | A552, A553, A554, A555, A558, A559, A560, A561, A564, |
| | A566, A567, A570, A571, A572, A573, A574, A575, A581, |
| | A583, A588, A591, A593, A595, A597, A600, A604, A607, |
| | A610, A618, A619, A623, A624, A625, A626, A630, A632, |
| | A634, A635, A636, A640, A659, A660, A670, A673, A677, |
| | A680, A685, A688, A689, A690, A694, A695, A699, A700, |
| | A706, A713, A714, A717, A725, A728, A729, A730, A731, |
| | A732, A738 |
| ++++ | A023, A025, A026, A050, A053, A060, A061, A062, A080, |
| | A084, A085, A102, A113, A136, A143, A151, A160, A168, |
| | A202, A210, A211, A226, A227, A242, A278, A290, A301, |
| | A302, A303, A331, A335, A339, A343, A345, A346, A347, |
| | A348, A352, A359, A362, A363, A364, A370, A371, A376, |
| | A380, A381, A391, A392, A403, A427, A442, A447, A461, |
| | A472, A481, A495, A496, A498, A531, A550, A562, A563, |
| | A565, A568, A569, A576, A579, A584, A585, A589, A590, |
| | A592, A601, A616, A627, A637, A638, A639, A641, A642, |
| | A645, A647, A652, A658, A666, A674, A691, A692, A693, |
| | A703, A705, A707, A708, A716, A720, A721, A722, A723, |
| | A726, A727, A733, A735 |
| +++++ | A170, A171, A205, A240, A243, A248, A285, A304, A304, |
| | A313, A316, A329, A355, A374, A375, A377, A378, A482, |
| | A486, A507, A587, A599, A613, A614, A615, A617, A628, |
| | A643, A644, A646, A648, A649, A657, A663, A675, A715, |
| | A724 |

TABLE 14
NRAS G12C FRET data

| IC50* | Examples |
|---|---|
| + | A038, A075, A076, A088, A095, A096, A107, A108, A112, |
| | A116, A120, A121, A123, A129, A130, A131, A131, A132, |
| | A133, A133, A135, A140, A141, A145, A155, A157, A162, |
| | A167, A175, A176, A182, A183, A184, A194, A196, A209, |
| | A244, A245, A246, A247, A247, A250, A255, A257, A266, |
| | A268, A270, A290, A292, A294, A297, A298, A299, A300, |
| | A316, A316, A316, A323, A324, A326, A333, A334, A336, |
| | A337, A339, A343, A348, A351, A365, A375, A376, A377, |
| | A380, A388, A391, A407, A411, A414, A427, A435, A474, |
| | A475, A479, A509, A527, A529, A534, A537, A542, A543, |
| | A544, A550, A551, A556, A577, A578, A579, A591, A600, |
| | A601, A609, A612, A616, A620, A626, A627, A628, A638, |
| | A642, A647, A658, A660, A663, A667, A673, A684, A689, |
| | A691, A692, A699, A705, A707, A711, A721, A727, A735 |
| ++ | A016, A054, A069, A103, A104, A105, A115, A121, A124, |
| | A126, A127, A128, A132, A133, A134, A135, A138, A142, |
| | A144, A148, A149, A150, A152, A153, A154, A155, A158, |
| | A164, A165, A166, A172, A178, A179, A181, A182, A183, |
| | A186, A187, A189, A191, A196, A200, A204, A207, A233, |
| | A237, A241, A241, A250, A251, A251, A252, A256, A260, |
| | A261, A262, A263, A264, A265, A267, A269, A275, A281, |
| | A291, A295, A296, A305, A317, A319, A322, A328, A332, |
| | A334, A338, A344, A346, A349, A350, A352, A353, A354, |
| | A361, A362, A363, A367, A368, A370, A371, A379, A381, |
| | A382, A383, A384, A385, A386, A387, A389, A394, A396, |
| | A397, A400, A403, A410, A417, A418, A419, A421, A423, |
| | A425, A432, A433, A436, A437, A438, A439, A440, A443, |
| | A444, A445, A448, A452, A454, A456, A458, A459, A463, |
| | A464, A465, A466, A476, A477, A478, A485, A489, A490, |
| | A491, A492, A493, A499, A501, A503, A504, A505, A510, |
| | A513, A515, A517, A518, A519, A525, A526, A528, A532, |
| | A533, A536, A538, A545, A549, A552, A558, A559, A560, |
| | A566, A567, A569, A572, A573, A574, A575, A580, A581, |
| | A582, A584, A585, A587, A589, A590, A592, A594, A595, |
| | A596, A597, A598, A599, A602, A603, A610, A614, A615, |
| | A617, A621, A622, A624, A629, A631, A632, A633, A634, |
| | A636, A637, A643, A644, A645, A646, A649, A650, A651, |
| | A653, A654, A657, A659, A661, A662, A664, A665, A668, |
| | A669, A671, A672, A674, A675, A676, A678, A679, A681, |
| | A682, A683, A685, A686, A695, A697, A698, A700, A701, |
| | A703, A704, A706, A709, A710, A712, A715, A719, A720, |
| | A722, A724, A726, A730, A733, A734, A736, A737 |
| +++ | A065, A075, A094, A109, A111, A112, A113, A115, A117, |
| | A118, A119, A120, A137, A139, A143, A144, A145, A146, |
| | A147, A151, A156, A159, A161, A167, A169, A173, A177, |
| | A180, A181, A184, A185, A188, A190, A192, A193, A194, |
| | A195, A197, A199, A201, A206, A209, A210, A211, A212, |
| | A214, A225, A226, A227, A232, A238, A249, A253, A254, |
| | A258, A258, A259, A261, A277, A278, A280, A281, A282, |
| | A283, A301, A302, A306, A310, A314, A320, A321, A327, |
| | A330, A331, A335, A340, A341, A342, A347, A356, A357, |
| | A358, A359, A360, A364, A366, A369, A372, A373, A374, |
| | A378, A390, A392, A393, A395, A399, A402, A404, A406, |
| | A408, A409, A412, A413, A415, A416, A420, A422, A424, |
| | A430, A431, A442, A446, A447, A449, A451, A455, A457, |
| | A461, A462, A467, A468, A469, A470, A471, A472, A473, |
| | A481, A483, A486, A487, A488, A494, A495, A497, A498, |
| | A502, A506, A507, A508, A511, A512, A520, A522, A524, |
| | A530, A531, A539, A540, A541, A546, A547, A548, A553, |
| | A554, A555, A557, A561, A562, A563, A564, A565, A568, |
| | A570, A571, A576, A583, A586, A588, A593, A604, A605, |
| | A606, A607, A608, A611, A618, A619, A623, A625, A630, |
| | A635, A639, A640, A641, A648, A652, A655, A656, A670, |
| | A677, A680, A687, A688, A690, A693, A694, A696, A702, |
| | A708, A713, A714, A716, A717, A718, A723, A725, A728, |
| | A729, A731, A732, A738, A739 |
| ++++ | A024, A102, A106, A110, A124, A125, A136, A160, A163, |
| | A168, A170, A171, A174, A186, A187, A191, A192, A202, |
| | A203, A205, A208, A210, A211, A212, A213, A228, A229, |
| | A235, A240, A242, A248, A274, A276, A279, A283, A284, |
| | A289, A303, A307, A309, A313, A325, A328, A345, A398, |
| | A401, A426, A441, A453, A460, A480, A484, A496, A514, |
| | A516, A521, A523, A535, A613, A666 |
| +++++ | A114, A122, A148, A198, A210, A210, A211, A211, A213, |
| | A214, A214, A225, A225, A230, A231, A231, A231, A234, |
| | A236, A239, A239, A243, A271, A272, A273, A279, A279, |
| | A283, A285, A286, A287, A288, A293, A304, A304, A304, |
| | A304, A308, A311, A312, A318, A318, A329, A355, A405, |
| | A428, A429, A450, A482, A500 |

TABLE 15
NRAS Q61R FRET data

| IC50* | Examples |
|---|---|
| + | A388 |
| ++ | A367, A368, A382, A383, A385, A386, A387, A389, A407, |
| | A417, A491, A492, A493, A542, A577, A594, A596, A598, |
| | A602, A603, A609, A611, A612, A621, A629, A633, A655, |
| | A664, A665, A667, A669, A678, A679, A697, A704, A712, |
| | A719, A734, A736, A737 |
| +++ | A012, A022, A075, A076, A082, A087, A105, A148, A167, |
| | A181, A207, A262, A275, A281, A297, A299, A300, A314, |
| | A333, A338, A349, A350, A353, A354, A356, A357, A360, |
| | A361, A369, A379, A384, A393, A394, A416, A463, A502, |
| | A537, A538, A539, A541, A543, A544, A545, A546, A548, |
| | A554, A555, A570, A571, A572, A573, A575, A578, A580, |
| | A581, A582, A583, A586, A591, A593, A595, A597, A604, |
| | A605, A606, A607, A608, A610, A618, A619, A620, A622, |
| | A624, A625, A630, A631, A632, A634, A635, A636, A650, |
| | A651, A653, A654, A656, A659, A661, A662, A668, A670, |
| | A671, A672, A676, A677, A680, A681, A682, A683, A684, |
| | A685, A686, A687, A688, A690, A695, A696, A698, A699, |
| | A700, A701, A702, A706, A709, A710, A711, A713, A714, |
| | A717, A718, A728, A732, A738 |
| ++++ | A016, A064, A074, A077, A083, A086, A089, A104, A115, |
| | A166, A168, A193, A252, A277, A290, A328, A330, A332, |
| | A335, A339, A340, A341, A342, A343, A344, A347, A351, |
| | A352, A358, A359, A363, A364, A365, A366, A370, A372, |
| | A373, A380, A381, A390, A391, A392, A442, A461, A472, |
| | A474, A475, A481, A494, A495, A496, A498, A503, A504, |
| | A505, A522, A531, A534, A540, A547, A549, A550, A551, |
| | A552, A553, A557, A558, A559, A560, A561, A564, A566, |
| | A567, A574, A588, A600, A601, A623, A626, A638, A639, |
| | A640, A641, A647, A652, A658, A660, A673, A689, A691, |
| | A693, A694, A720, A722, A725, A727, A729, A730, A731, |
| | A739 |
| +++++ | A023, A025, A026, A045, A050, A053, A060, A061, A062, |
| | A080, A084, A085, A102, A113, A136, A143, A151, A160, |
| | A170, A171, A172, A202, A205, A210, A211, A226, A227, |
| | A240, A242, A243, A248, A278, A285, A301, A302, A303, |
| | A304, A304, A313, A316, A329, A331, A345, A346, A348, |
| | A355, A362, A371, A374, A375, A376, A377, A378, A403, |
| | A427, A447, A468, A482, A486, A507, A556, A562, A563, |
| | A565, A568, A569, A576, A579, A584, A585, A587, A589, |
| | A590, A592, A599, A613, A614, A615, A616, A617, A627, |
| | A628, A637, A642, A643, A644, A645, A646, A648, A649, |
| | A657, A663, A666, A674, A675, A692, A703, A705, A707, |
| | A708, A715, A716, A721, A723, A724, A726, A733, A735 |

TABLE 16
NRAS Q61K FRET data

| IC50* | Examples |
|---|---|
| + | A388 |
| ++ | A262, A297, A299, A338, A349, A350, A353, A354, A367, |
| | A368, A369, A382, A383, A384, A385, A386, A387, A389, |
| | A394, A407, A416, A417, A463, A491, A492, A493, A542, |
| | A543, A544, A545, A577, A580, A582, A594, A596, A598, |
| | A602, A603, A609, A611, A612, A620, A621, A622, A629, |
| | A631, A633, A650, A651, A653, A654, A655, A661, A662, |
| | A664, A665, A667, A668, A669, A671, A678, A679, A681, |
| | A682, A683, A686, A687, A696, A697, A698, A701, A702, |
| | A704, A709, A710, A711, A712, A718, A719, A734, A736, |
| | A737, A739 |
| +++ | A012, A022, A074, A075, A076, A077, A082, A086, A087, |
| | A105, A148, A167, A181, A207, A275, A277, A281, A300, |
| | A314, A330, A333, A340, A356, A360, A361, A379, A390, |
| | A393, A502, A537, A538, A539, A541, A546, A547, A548, |
| | A554, A555, A561, A570, A571, A572, A573, A574, A575, |
| | A578, A581, A583, A586, A588, A593, A595, A597, A604, |
| | A605, A606, A607, A608, A610, A618, A619, A623, A624, |
| | A625, A630, A632, A634, A635, A636, A656, A660, A670, |
| | A672, A676, A677, A680, A684, A685, A688, A690, A694, |
| | A695, A699, A700, A706, A713, A714, A717, A728, A729, |
| | A730, A731, A732, A738 |
| ++++ | A016, A025, A045, A050, A060, A061, A064, A080, A083, |
| | A089, A104, A115, A143, A151, A166, A168, A172, A193, |
| | A202, A210, A211, A227, A252, A278, A290, A328, A331, |
| | A332, A335, A339, A341, A342, A343, A344, A347, A351, |
| | A352, A357, A358, A359, A363, A364, A365, A366, A370, |
| | A372, A373, A380, A381, A391, A427, A442, A461, A468, |
| | A472, A474, A475, A481, A494, A495, A496, A498, A503, |
| | A504, A505, A522, A531, A534, A540, A549, A550, A551, |
| | A552, A553, A557, A558, A559, A560, A562, A563, A564, |
| | A566, A567, A576, A590, A591, A600, A601, A626, A637, |
| | A638, A639, A640, A641, A647, A652, A659, A673, A689, |
| | A691, A693, A705, A720, A722, A725, A727, A735 |
| +++++ | A023, A026, A053, A062, A084, A085, A102, A113, A136, |
| | A160, A170, A171, A205, A226, A240, A242, A243, A248, |
| | A285, A301, A302, A303, A304, A304, A313, A316, A329, |
| | A345, A346, A348, A355, A362, A371, A374, A375, A376, |
| | A377, A378, A392, A403, A447, A482, A486, A507, A556, |
| | A565, A568, A569, A579, A584, A585, A587, A589, A592, |
| | A599, A613, A614, A615, A616, A617, A627, A628, A642, |
| | A643, A644, A645, A646, A648, A649, A657, A658, A663, |
| | A666, A674, A675, A692, A703, A707, A708, A715, A716, |
| | A721, A723, A724, A726, A733 |

TABLE 17
NRAS WT FRET data

| IC50* | Examples |
|---|---|
| + | A388 |
| ++ | A075, A076, A087, A167, A262, A275, A297, A299, A300, |
| | A333, A338, A349, A367, A368, A382, A383, A385, A386, |
| | A387, A389, A407, A417, A491, A492, A493, A537, A542, |
| | A577, A594, A596, A598, A602, A603, A605, A609, A612, |
| | A621, A629, A633, A664, A665, A667, A669, A679, A697, |
| | A704, A709, A711, A712, A719, A734, A736, A737 |
| +++ | A012, A016, A022, A074, A077, A082, A086, A105, A148, |
| | A166, A181, A207, A252, A277, A281, A340, A350, A351, |
| | A353, A354, A356, A357, A358, A360, A361, A369, A379, |
| | A384, A393, A394, A416, A463, A502, A538, A539, A541, |
| | A543, A544, A545, A546, A547, A548, A550, A552, A554, |
| | A555, A557, A558, A564, A570, A571, A572, A573, A575, |
| | A578, A580, A581, A582, A583, A586, A591, A593, A595, |
| | A600, A604, A606, A607, A608, A610, A611, A618, A620, |
| | A622, A624, A625, A630, A631, A632, A634, A635, A636, |
| | A647, A650, A651, A653, A654, A655, A656, A659, A660, |
| | A661, A662, A668, A670, A671, A672, A676, A677, A678, |
| | A680, A681, A682, A683, A684, A685, A686, A687, A688, |
| | A690, A695, A696, A698, A699, A700, A701, A702, A710, |
| | A713, A714, A717, A718, A722, A728, A729, A730, A731, |
| | A732, A738, A739 |
| ++++ | A025, A045, A060, A061, A062, A064, A083, A089, A104, |
| | A115, A151, A168, A172, A193, A202, A210, A211, A227, |
| | A278, A290, A314, A328, A330, A331, A332, A335, A339, |
| | A341, A342, A343, A344, A347, A348, A352, A359, A362, |
| | A363, A364, A365, A366, A370, A371, A372, A373, A375, |
| | A376, A378, A380, A381, A390, A391, A392, A427, A447, |
| | A461, A468, A472, A474, A475, A481, A494, A495, A496, |
| | A498, A503, A504, A505, A507, A522, A531, A534, A540, |
| | A549, A551, A553, A556, A559, A560, A561, A562, A563, |
| | A566, A567, A574, A576, A579, A584, A585, A588, A589, |
| | A590, A597, A601, A616, A619, A623, A626, A637, A638, |
| | A639, A640, A641, A642, A645, A648, A652, A658, A673, |
| | A689, A691, A693, A694, A703, A705, A706, A707, A708, |
| | A716, A720, A721, A723, A725, A726, A727, A735 |
| +++++ | A023, A026, A050, A053, A080, A084, A085, A102, A113, |
| | A136, A143, A160, A170, A171, A205, A226, A240, A242, |
| | A243, A248, A285, A302, A303, A304, A304, A313, A316, |
| | A329, A345, A346, A355, A374, A377, A403, A442, A482, |
| | A486, A565, A568, A569, A587, A592, A599, A613, A614, |
| | A615, A617, A627, A628, A643, A644, A646, A649, A657, |
| | A663, A666, A674, A675, A692, A715, A724, A733 |

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC50).

The assay took place over 7 days. On day 1, cells in 2D culture were harvested during logarithmic growth and suspended in culture medium at 1×105 cells/ml. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% CO₂. On day 2, one plate (for t0 reading) was removed and 10 μl growth medium plus 100 μl CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 37C and 5% CO₂. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC50 for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite8 having a KRAS G12C mutation, is insensitive to the KRAS G12C(OFF) inhibitor MRTX-849 (Hallin et al., Cancer Discovery 10:54-71 (2020)), and the cell line SW1573, which is insensitive to the KRAS G12C(OFF) inhibitor AMG510 (Canon et al., Nature 575:217-223 (2019)).

TABLE 18
IC50 values for various cancer cell lines with Compound B

| Cell Line | Histotype | Mutant | IC50* |
|---|---|---|---|
| NCI-H358 | Lung | KRAS G12C | very sensitive |
| MIA PaCa-2 | Pancreas | KRAS G12C | very sensitive |
| SW837 | Intestine/Large/ | KRAS G12C | very sensitive |
| | Colorectum | | |
| KYSE-410 | HN/Esophagus | KRAS G12C | moderately |
| | | | sensitive |
| NCI-H727 | Lung | KRAS G12V | moderately |
| | | | sensitive |
| OVCAR-5 | Ovary | KRAS G12V | moderately |
| | | | sensitive |
| Capan-2 | Pancreas | KRAS G12V | moderately |
| | | | sensitive |
| NCI-H747 | Intestine/Large/ | other KRAS (G13D) | moderately |
| | Colorectum | | sensitive |
| NCI-H441 | Lung | KRAS G12V | moderately |
| | | | sensitive |
| HEC-1-A | Uterus | KRAS G12D | moderately |
| | | | sensitive |
| NOZ | Liver/Bile duct | KRAS G12V | moderately |
| | | | sensitive |
| HCT116 | Intestine/Large/ | other KRAS (G13D) | moderately |
| | Colorectum | | sensitive |
| Calu-6 | Lung | other KRAS (Q61K) | moderately |
| | | | sensitive |
| HuCCT1 | Liver/Bile duct | KRAS G12D | moderately |
| | | | sensitive |
| NCI-H2009 | Lung | other KRAS (G12A) | low sensitivity |
| NCI-H1975 | Lung | other MAPK (EGFR | low sensitivity |
| | | T790M, L858R) | |
| SW1573 | Lung | KRAS G12C | not sensitive |
| AGS | Stomach | KRAS G12D | low sensitivity |
| AsPC-1 | Pancreas | KRAS G12D | low sensitivity |
| SNU-668 | Stomach | other KRAS (Q61K) | low sensitivity |
| HPAC | Pancreas | KRAS G12D | low sensitivity |
| NCI-H1838 | Lung | other MAPK (NF1 | low sensitivity |
| | | mut) | |
| NCI-H3122 | Lung | other MAPK (EML4- | low sensitivity |
| | | ALK(E13, A20)) | |
| NCI-H460 | Lung | other KRAS (Q61H) | low sensitivity |
| SW403 | Intestine/Large/ | KRAS G12V | low sensitivity |
| | Colorectum | | |
| A549 | Lung | other KRAS (G12S) | low sensitivity |
| CAL-62 | HN/Thyroid | other KRAS (G12R) | low sensitivity |
| DV-90 | Lung | other KRAS (G13D) | low sensitivity |
| OZ | Liver/Bile duct | other KRAS (Q61L) | low sensitivity |
| A-375 | Skin | BRAF V600E | low sensitivity |
| BxPC-3 | Pancreas | other MAPK (BRAF | low sensitivity |
| | | V487_P492delinsA) | |
| SW48 | Intestine/Large/ | not MAPK (PIK3CA | low sensitivity |
| | Colorectum | G914R, EGFR | |
| | | G719S) | |
| HCC1588 | Lung | KRAS G12D | low sensitivity |
| TOV-21G | Ovary | other KRAS (G13C) | low sensitivity |
| SW948 | Intestine/Large/ | other KRAS (Q61L) | low sensitivity |
| | Colorectum | | |
| LS513 | Intestine/Large/ | KRAS G12D | low sensitivity |
| | Colorectum | | |
| MeWo | Skin | other MAPK (NF1 | low sensitivity |
| | | mut) | |

*Key:
low sensitivity: IC50 ≥ 1 uM
moderately sensitive: 1 uM > IC50 ≥ 0.1 uM
very sensitive: IC50 < 0.1 uM

TABLE 19
Summary of IC50 results for various cancer cell lines with several
compounds of the present invention (Compounds B and E-M)

| Cell Line | Histotype | Mutant | E | F | G | H | I | J | K | L | B | M |
|---|---|---|---|---|---|---|---|---|---|---|---|---|
| SW837 | Intestine/ | KRAS G12C | V | V | V | V | V | V | V | V | V | V |
| | Large/ | | | | | | | | | | | |
| | Colorectum | | | | | | | | | | | |
| MIA | Pancreas | KRAS G12C | V | V | V | V | V | V | V | V | V | V |
| PaCa-2 | | | | | | | | | | | | |
| KYSE- | HN/ | KRAS G12C | L | L | L | L | L | L | L | L | L | L |
| 410 | Esophagus | | | | | | | | | | | |
| H358 | Lung | KRAS G12C | V | V | V | V | V | V | V | V | V | V |
| H2122 | Lung | KRAS G12C | V | V | V | V | V | V | V | V | V | V |
| H1373 | Lung | KRAS G12C | V | V | V | V | V | V | V | V | V | V |
| H23 | Lung | KRAS G12C | V | V | V | V | V | V | V | V | V | V |
| H1792 | Lung | KRAS G12C | V | V | V | V | V | V | V | V | V | V |
| AsPC-1 | Pancreas | KRAS G12D | L | L | M | L | L | M | L | L | M | L |
| A375 | Skin | BRAF | L | L | L | L | L | L | L | L | L | L |
| | | V600E | | | | | | | | | | |
| H1975 | Lung | other | M | L | M | L | L | M | L | M | M | L |
| | | MAPK | | | | | | | | | | |
| | | (EGFR | | | | | | | | | | |
| | | T790M, | | | | | | | | | | |
| | | L858R) | | | | | | | | | | |
| HCC1588 | Lung | KRAS G12D | L | L | L | L | L | L | L | L | L | L |
| H441 | Lung | KRAS G12V | M | M | M | L | M | V | L | M | V | L |

*Key:
(L) low sensitivity: IC50 ≥ 1 uM
(M) moderately sensitive: 1 uM > IC50 ≥ 0.1 uM
(V) very sensitive: IC50 < 0.1 uM

### In Vivo NSCLC K-Ras G12C Xenograft Models

### Compound A:

### Methods:

The effects of a compound of the present invention, Compound A (H358 pERK K-Ras G12C EC50: 0.001 μM), on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×10⁶ cells/mouse) subcutaneously in the flank. At the indicated tumor volume (dotted line, FIG. 2A), mice were randomized to treatment groups to start the administration of test articles or vehicle. Compound A was administered by oral gavage daily at the dose of 100 mg/kg. Body weight and tumor volume (using calipers) was measured twice weekly until study endpoints. Spaghetti plot (FIG. 2B) shows the tumor volume change in individual tumors during the course of treatment.

### Results:

FIG. 2A shows Compound A dosed at 100 mg/kg by daily oral gavage led to tumor regression in NCI-H358 KRASG12C xenograft model, which is a sensitive model to KRASG12C inhibition alone. The spaghetti titer plot (FIG. 2B) displaying individual tumor growth is shown next to the tumor volume plot (FIG. 2A). Over the treatment course of 28 days, Compound A drove tumor regression in all 10 animals bearing NCI-H358 KRASG12C tumors.

### Compound B:

### Methods:

The combinatorial effect of a compound of the present invention, Compound B (H358 pERK K-Ras G12C EC50: 0.003 μM), with cobimetinib on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×10⁶ cells/mouse) subcutaneously in the flank. At indicated tumor volume (dotted line, FIG. 3A), mice were randomized to treatment groups to start the administration of test articles or vehicle. Compound B was administered by intermittent (twice weekly) intravenous injection at the dose of 50 mg/kg. Cobimetinib was administered by daily oral gavage at 2.5 mg/kg. The combination of Compound B and cobimetinib at their respective single-agent dose and regimen was also tested. Body weight and tumor volume (using calipers) was measured twice weekly until study endpoints. End of study responses in individual tumors were plotted as a waterfall plot (FIG. 3B), and the numbers indicate number of tumor regression in each group. Tumor regression is defined as greater than 10% reduction of tumor volume at the end of study relative to initial volume.

### Results:

FIG. 3A shows the combination of intermittent intravenous administration of Compound B at 50 mg/kg plus daily oral administration of cobimetinib at 2.5 mg/kg drove tumor regression, whereas each single agent led to tumor growth inhibition. End of study responses were shown as waterfall plots (FIG. 3B), which indicate 6 out 10 mice had tumor regression in the combination group, whereas no tumor regressions recorded in each single agent group.

### Compound C:

### Methods:

The combinatorial effect of a compound of the present invention, Compound C(H358 pERK K-Ras G12C EC50: 0.007 μM), with a SHP2 inhibitor, RMC-4550, on tumor cell growth in vivo were evaluated in the human non-small cell lung cancer NCI-H358 KRASG12C xenograft model using female BALB/c nude mice (6-8 weeks old). Mice were implanted with NCI-H358 tumor cells in 50% Matrigel (5×10⁶ cells/mouse) subcutaneously in the flank. At indicated tumor volume (dotted line, FIG. 4A), mice were randomized to treatment groups to start the administration of test articles or vehicle. Compound C was administered by once weekly intravenous injection at the dose of 60 mg/kg. SHP2 inhibitor was administered by daily oral gavage at 30 mg/kg. The combination of Compound C and SHP2 inhibitor at their respective single-agent dose and regimen was also tested. Body weight and tumor volume (using calipers) was measured twice weekly until study endpoints. End of study responses in individual tumors were plotted as a waterfall plot (FIG. 4B), and the numbers indicate number of tumor regression in each group. Tumor regression is defined as greater than 10% reduction of tumor volume at the end of study relative to initial volume.

### Results:

In FIG. 4A, the combinatorial activity of once weekly intravenous administration of Compound C at 60 mg/kg plus daily oral administration of SHP2 inhibitor at 30 mg/kg is shown. The combination treatment had similar anti-tumor activity as the single agent SHP2 inhibitor, but the combination treatment led to 8 out of 10 mice with tumor regression, whereas single agent SHP2 inhibitor led to 5 out of 10 mice with tumor regressions. Single agent Compound C administered once weekly via intravenous injection led to tumor growth inhibition with one tumor regression.

### Cell Proliferation Assay

### Methods:

NCI-H358 cells were plated in 12-well tissue culture plates at a density of 100,000 cells/well in RPMI 1640 (10% FBS, 1% PenStrep) and cultured overnight at 37° C., 5% CO₂. The following day, cells were treated with either trametinib (10 nM) or a compound of the present invention, Compound D (H358 pERK K-Ras G12C EC50: 0.024 μM), (17 nM). These concentrations represent the EC50 values from a 72-hour proliferation assay using the CellTiter-Glo® reagent (Promega). Additionally, cells were treated with the combination of trametinib and Compound D at the above indicated concentrations. The plate was placed in the Incucyte S3 live cell analysis system (37° C., 5% CO₂) and confluence was measured by recording images at 6-hour intervals for a maximum of 40 days, or until wells reached maximal confluence. Media and drug were replaced at 3-4 day intervals. Data are plotted as % confluence over the time course of the experiment for each single agent and respective combination (FIG. 5).

### Results:

As shown in FIG. 5, treatment of NCI-H358 cells with submaximal (EC50) concentrations of Compound D or MEK inhibitor results in a short period of growth inhibition, followed by proliferation. Cells reach maximal confluence in ˜10 days after the addition of drug. The combination of the MEK inhibitor, trametinib, with Compound D resulted in complete and sustained inhibition of cell growth throughout the duration of the assay.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX C

Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

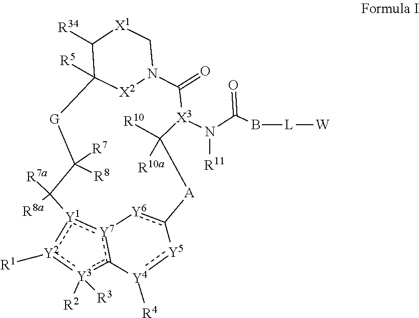

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

Also provided are pharmaceutical compositions comprising a compound of Formula I, or a pharmaceutically acceptable salt thereof, and a pharmaceutically acceptable excipient.

Further provided is a conjugate, or salt thereof, comprising the structure of Formula IV:

M-L-P Formula IV
wherein L is a linker;
P is a monovalent organic moiety; and
M has the structure of Formula V:

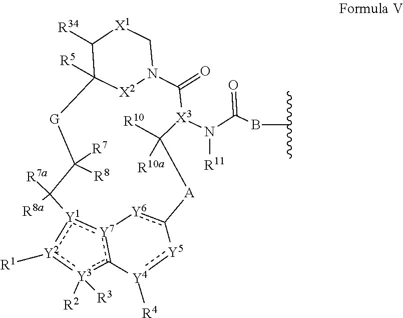

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1A: A compound of the present invention, Compound A, deeply and durably inhibits oncogenic signals in a pancreatic CDX model (HPAC CDX model, PDAC, KRAS G12D/WT). Single dose experiment, n=3/time point, all dose levels well tolerated.

FIG. 1B: Treatment of KRAS G12D tumors in vivo with a compound of the present invention, Compound A, drives tumor regressions in a pancreatic CDX model (HPAC CDX model, PDAC, KRAS G12D/WT). n=10/group, *** p<0.001. All dose levels well tolerated.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value). As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, ¹H- and ³H-imidazole, ¹H—, ²H— and ⁴H-1,2,4-triazole, ¹H- and ²H-isoindole, and ¹H- and ²H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium;
halogen; —(CH₂)₀₋₄R^{∘}; —(CH₂)₀₋₄OR^{∘}; —O(CH₂)₀₋₄R^{∘};
—O—(CH₂)₀₋₄C(O)OR^{∘}; —(CH₂)₀₋₄CH (OR^{∘})₂; —(CH₂)₀₋₄SR^{∘}; —(CH₂)₀₋₄Ph, which may be substituted with R^{∘}; —(CH₂)₀₋₄O (CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4 to 8-membered saturated or unsaturated heterocycloalkyl (e.g., pyridyl); 3 to 8-membered saturated or unsaturated cycloalkyl (e.g., cyclopropyl, cyclobutyl, or
cyclopentyl); —NO₂; —CN; —N₃; —(CH₂)₀₋₄N(R^{∘}))₂; —(CH₂)₀₋₄N(R^{∘}C(O)R^{∘};
—N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N (R^{∘})C(O)R^{∘}; —N(R^{∘})N(R^{∘})C(O)NR^{∘}₂; —N(R^{∘})N (R^{∘})C(O)OR^{∘}; —(CH₂)₀₋₄C(O)R^{∘}; —C(S)R^{∘}; —(CH₂)₀₋₄C(O)OR^{∘};)
—(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O) (CH₂)₀₋₄SR^{∘}; —SC(S)SR^{∘}; —(CH₂)₀₋₄SC(O)R^{∘}; —(CH₂)₀₋₄C(O)NR^{∘}₂;
—C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}); —N(R^{∘})S(O)₂NR^{∘)}₂; —N(R^{∘}S(O)₂R^{∘}; —N(OR^{∘})R^{∘}; —C(NOR^{∘}) NR^{∘}₂; —C(NH)NR^{∘}₂; —P(O)₂ R^{∘};
—P(O)R^{∘}₂; —P(O)(OR^{∘})₂; —OP(O)R^{∘}₂; —OP(O)(OR^{∘})₂; —OP(O)(OR^{∘}) R^{∘}, —SiR^{∘}₃; —(C₁-C₄ straight or branched) alkylene)O—N(R^{∘}₂; or —(C₁-C₄ straight or branched) alkylene) C(O)O—N(R^{∘}₂, wherein each R^{∘} may be substituted as defined below and is independently hydrogen, —C₁-C₆ aliphatic, —CH₂Ph, —O(CH₂)₀₋₁Ph, —CH₂-(5 to 6 membered heteroaryl ring), or a 3 to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3 to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•},
(haloR^{•}), —(CH₂)₀₋₂OH, —(CH₂)₀₋₂OR^{•}, —(CH₂)₀₋₂CH (OR^{•})₂; —O (haloR^{•}), —CN, —N₃, —(CH₂)₀₋₂C(O)R^{•}, —(CH₂)₀₋₂C(O)OH,
(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{∘}, —(C₁₋₄ straight or branched alkylene )C(O)OR′, or —SSR, wherein each R^{•} is unsubstituted or where preceded by “halo” is substituted only with one or more halogens, and is independently selected from C₁-C₄ aliphatic, —CH₂Ph, —O (CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur.

Suitable substituents on the aliphatic group of R* include
halogen, —R^{•}, -(haloR^{•}), —OH, —OR^{•},
—O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently
C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)Rt, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rt) S(O)₂R^{†}; wherein each R^{†} is independently hydrogen, C₁-C₆ aliphatic which may be substituted as defined below, unsubstituted —OPh, or an unsubstituted 3 to 6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3 to 12-membered saturated, partially unsaturated, or aryl mono- or bicyclic ring having 0-4 heteroatoms independently selected from nitrogen, oxygen, or sulfur.

Suitable substituents on an aliphatic group of Rt are independently halogen, —R^{•}, -(haloR^{•}), —OH, —OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

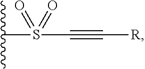

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by-N (C(O)—H)—.

The terms “carbocyclic” and “carbocyclyl,” as used herein, refer to a monovalent, optionally substituted 3 to 12-membered monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O) (OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused, or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused, or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

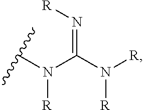

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent, monocyclic, bicyclic or polycyclic ring system, which may be bridged, fused, or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more-OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting moiety B to moiety W in a compound of Formula I, such that the resulting compound is capable of achieving an IC50 of 2 μM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras: RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

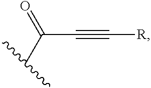

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### Detailed Description

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that both covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., the —CH₂—COOH or —CH₂—COO-side chain of the aspartic acid at position 12 or 13 of a mutant Ras protein). Covalent adducts may also be formed with other side chains of Ras. In addition or alternatively, non-covalent interactions may be at play: for example, van der Waals, hydrophobic, hydrophilic, and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art. One method of determining covalent adduct formation is to perform a “cross-linking” assay, such as described in the Examples, and below:
Note—the following protocol describes a procedure for monitoring cross-linking of K-Ras G12C (GMP-PNP) to a compound of the invention. This protocol may also be executed substituting other Ras proteins or nucleotides, such as K-Ras G12D.
The purpose of this biochemical assay is to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A and 2 μM test compound, a 5 μM stock of GMP-PNP-loaded K-Ras (1-169) G12C is diluted 10-fold to yield a final concentration of 0.5 μM; with final sample volume being 100 μL.
The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras.

Accordingly, provided herein is a compound, or pharmaceutically acceptable salt thereof, having the structure of Formula I:

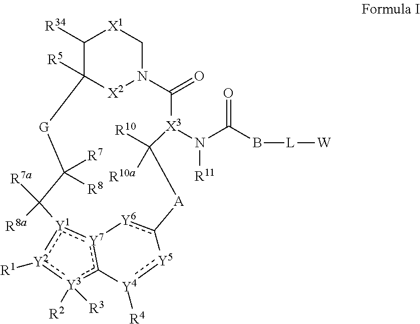

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl (e.g., methyl).

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, R³⁴ is hydrogen.

In some embodiments of compounds of the present invention, G is optionally substituted C₁-C₄ heteroalkylene.

In some embodiments, a compound of the present invention has the structure of Formula Ia, or a pharmaceutically acceptable salt thereof:

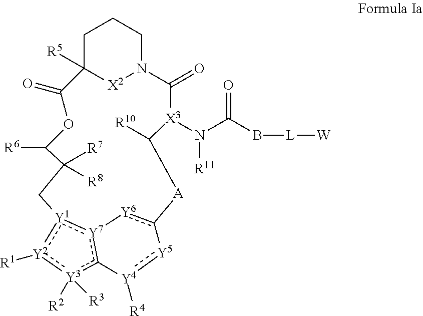

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, X² is NH. In some embodiments, X³ is CH.

In some embodiments of compounds of the present invention, R¹¹ is hydrogen. In some embodiments, R¹¹ is C₁-C₃ alkyl, such as methyl.

In some embodiments, a compound of the present invention has the structure of Formula Ib, or a pharmaceutically acceptable salt thereof:

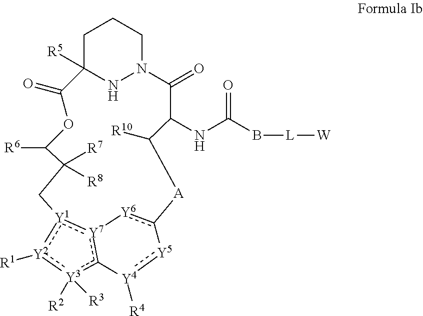

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an NV-ethoxycarbonyl-2-ethoxy-1,2-dihydroquinoline (EEDQ), an iso-EEDQ or other EEDQ derivative, an epoxide, an oxazolium, or a glycal;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene.

In some embodiments of compounds of the present invention, R⁴ is hydrogen.

In some embodiments of compounds of the present invention, R⁵ is hydrogen. In some embodiments, R⁵ is C₁-C₄ alkyl optionally substituted with halogen. In some embodiments, R⁵ is methyl.

In some embodiments of compounds of the present invention, Y⁴ is C. In some embodiments, R⁴ is hydrogen. In some embodiments, Y⁵ is CH. In some embodiments, Y⁶ is CH. In some embodiments, Y¹ is C. In some embodiments, Y² is C. In some embodiments, Y³ is N. In some embodiments, R³ is absent. In some embodiments, Y⁷ is C.

In some embodiments, a compound of the present invention has the structure of Formula Ic, or a pharmaceutically acceptable salt thereof:

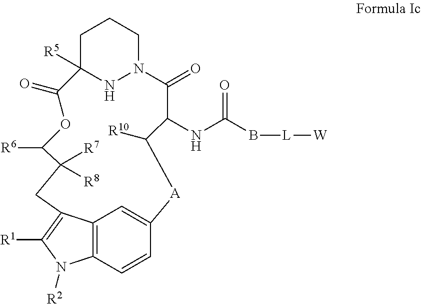

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, R⁶ is hydrogen.

In some embodiments of compounds of the present invention, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl.

In some embodiments of compounds of the present invention, R⁷ is optionally substituted C₁-C₃ alkyl. In some embodiments, R⁷ is C₁-C₃ alkyl.

In some embodiments of compounds of the present invention, R⁸ is optionally substituted C₁-C₃ alkyl. In some embodiments, R⁸ is C₁-C₃ alkyl.

In some embodiments, a compound of the present invention has the structure of Formula Id, or a pharmaceutically acceptable salt thereof:

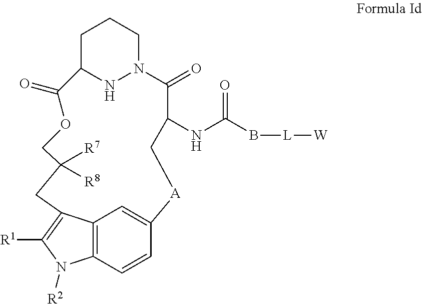

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of compounds of the present invention, R¹ is 5 to 10-membered heteroaryl. In some embodiments, R¹ is optionally substituted 6-membered aryl or optionally substituted 6-membered heteroaryl.

In some embodiments, a compound of the present invention has the structure of Formula Ie, or a pharmaceutically acceptable salt thereof:

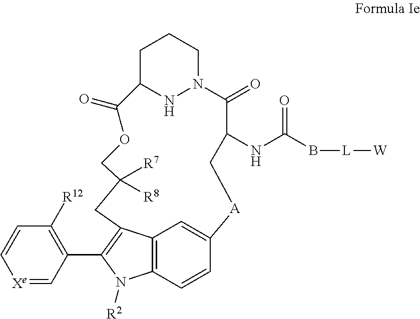

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} is N or CH; and
R¹² is optionally substituted C₁-C₆ alkyl or optionally substituted C₁-C₆ heteroalkyl.

In some embodiments of compounds of the present invention, X^{e} is N. In some embodiments, X^{e} is CH.

In some embodiments of compounds of the present invention, R¹² is optionally substituted C₁-C₆ heteroalkyl. In some embodiments, R¹² is

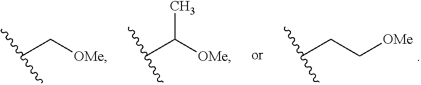

In some embodiments, R¹² is

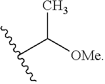 [SMILES: *C(C)OC]

In some embodiments, a compound of the present invention has the structure of Formula If, or a pharmaceutically acceptable salt thereof:

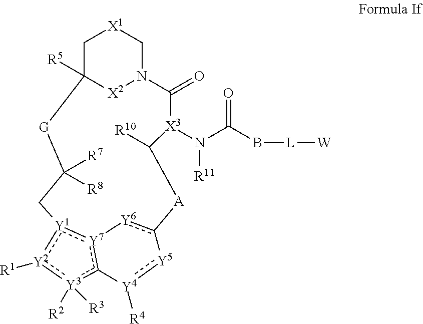

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O) NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or
a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, a compound of the present invention has the structure of Formula VI, or a pharmaceutically acceptable salt thereof:

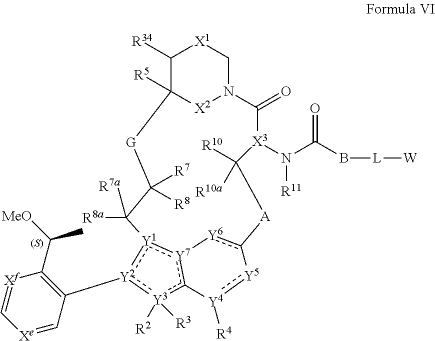

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R³⁴ is hydrogen or C₁-C₃ alkyl; and
X^{e} and X^{f} are, independently, N or CH.

In some embodiments, a compound of the present invention has the structure of Formula Va, or a pharmaceutically acceptable salt thereof:

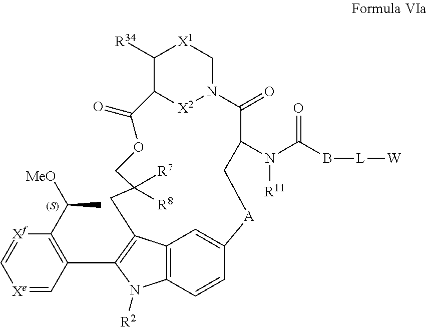

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of Formula VIb, or a pharmaceutically acceptable salt thereof:

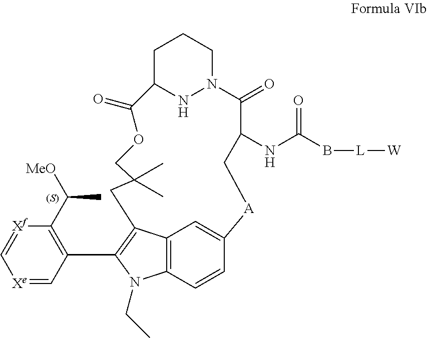

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of a compound of the present invention, X^{e} is N and X^{f} is CH. In some embodiments, X^{e} is CH and X^{f} is N.

In some embodiments, a compound of the present invention has the structure of Formula VII, or a pharmaceutically acceptable salt thereof:

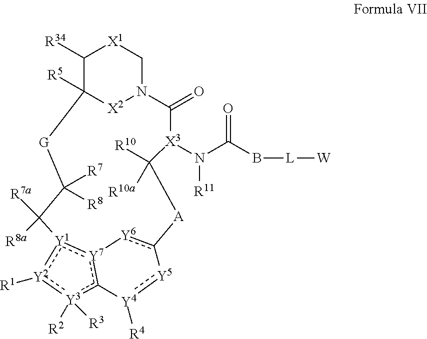

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O) NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is

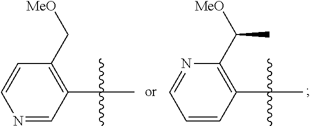 [SMILES: *c1cnccc1COC ; *c1cccnc1[C@H](C)OC]

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

In some embodiments of compounds of the present invention, A is optionally substituted 6-membered arylene. In some embodiments, A has the structure:

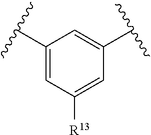

wherein R¹³ is hydrogen, hydroxy, amino, optionally substituted C₁-C₆ alkyl, or optionally substituted C₁-C₆ heteroalkyl. In some embodiments, R¹³ is hydrogen. In some embodiments, R¹³ is hydroxy.

In some embodiments of compounds of the present invention, B is —CHR⁹—. In some embodiments, R⁹ is optionally substituted C₁-C₆ alkyl or optionally substituted 3 to 6-membered cycloalkyl. In some embodiments, R⁹ is:

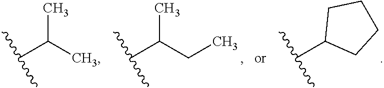

In some embodiments, R⁹ is:

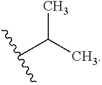 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, B is optionally substituted 6-membered arylene. In some embodiments, B is 6-membered arylene. In some embodiments, B is:

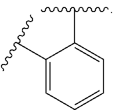 [SMILES: *c1ccccc1*]

In some embodiments of compounds of the present invention, R⁷ is methyl.

In some embodiments of compounds of the present invention, R⁸ is methyl.

In some embodiments, R³⁴ is hydrogen.

In some embodiments of compounds of the present invention, the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A². In some embodiments, the linker is acyclic. In some embodiments, the linker has the structure of Formula IIa:

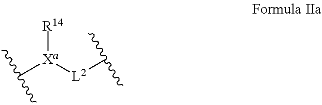

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

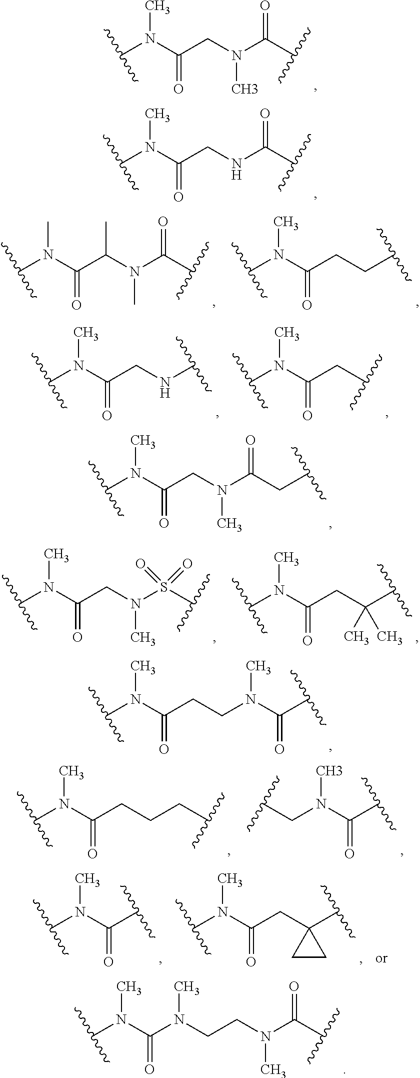

In some embodiments, the linker is or a comprises a cyclic group. In some embodiments, the linker has the structure of Formula IIb:

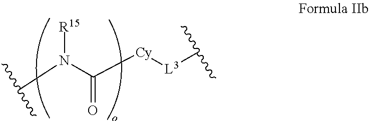

wherein o is 0 or 1;
R¹⁵ is hydrogen or optionally substituted C₁-C₆ alkyl; Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene. In some embodiments, the linker has the structure:

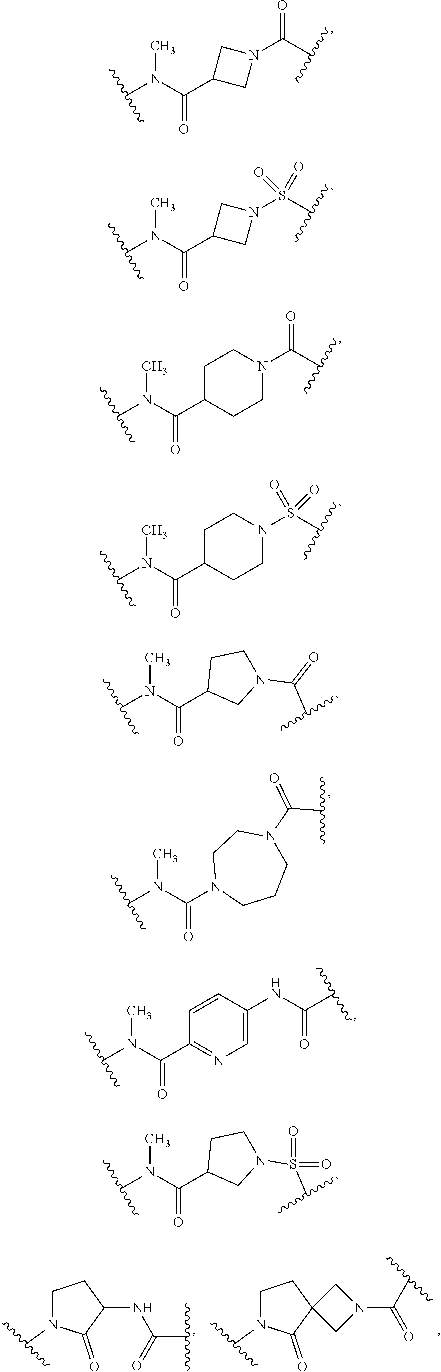

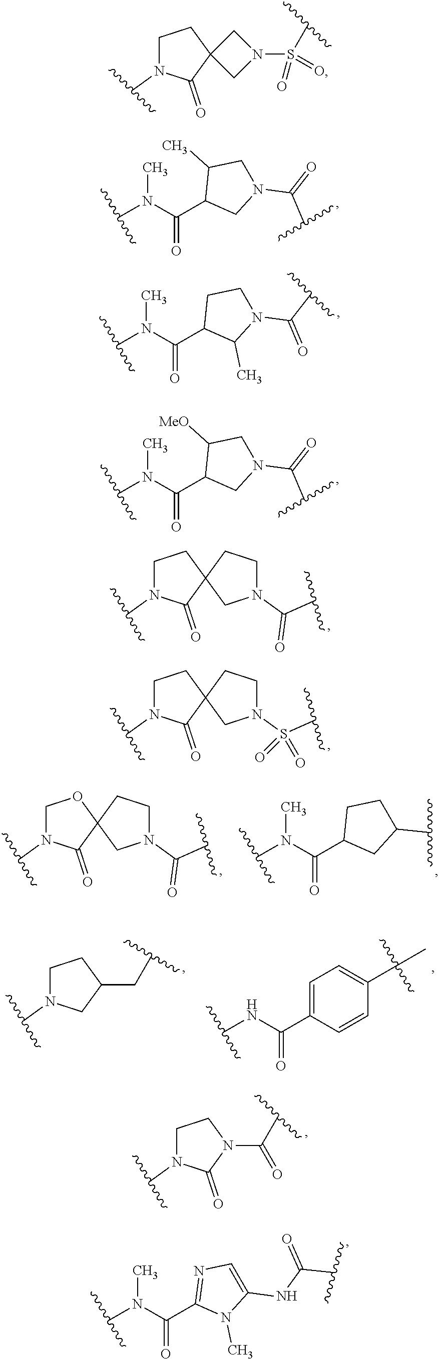

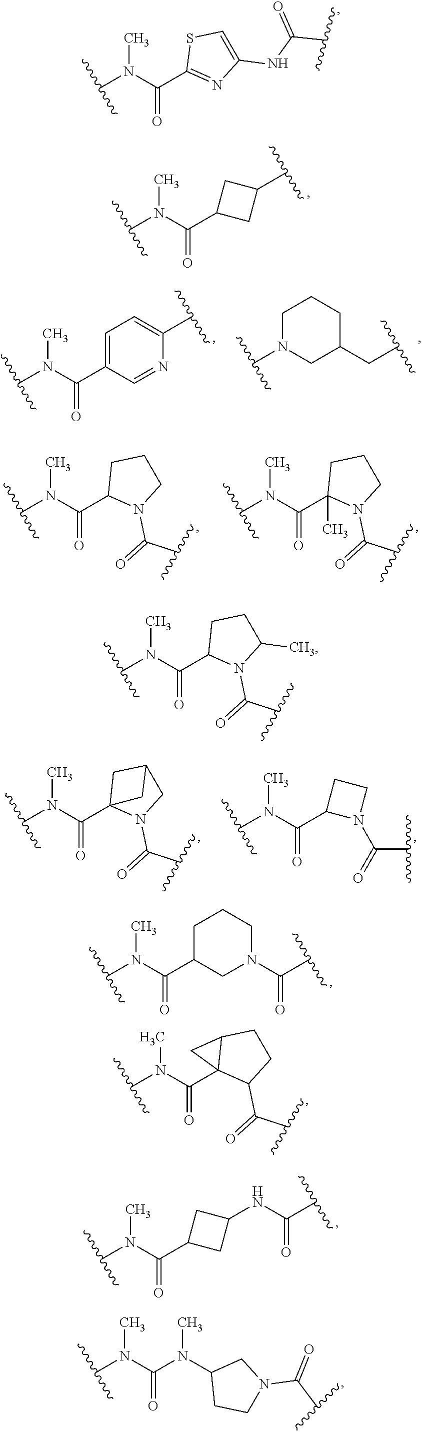

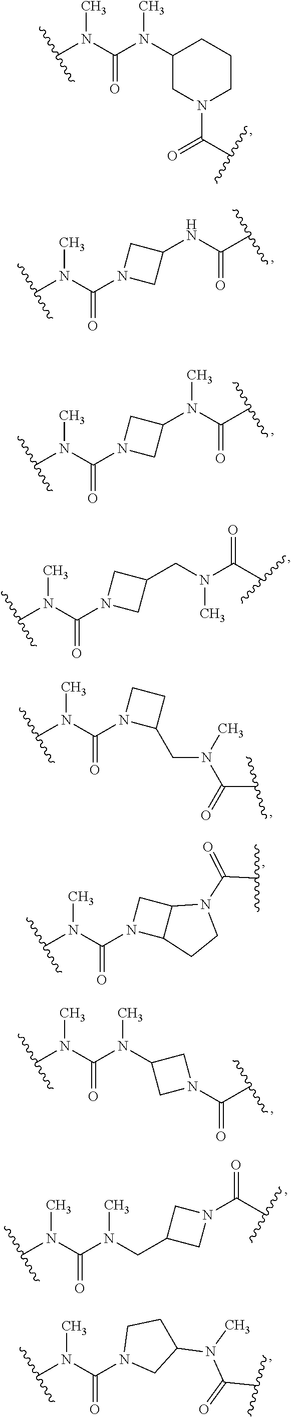

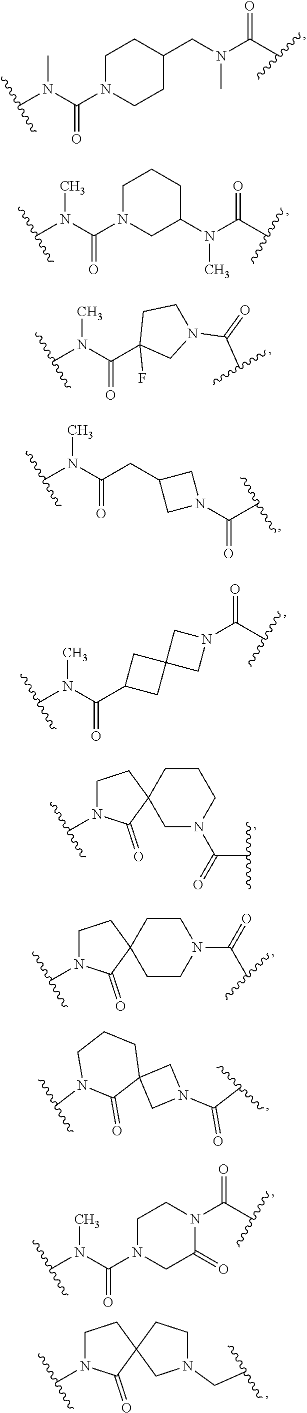

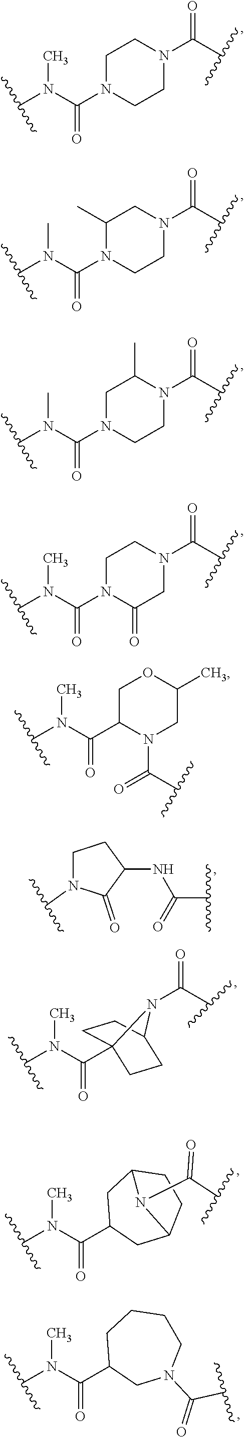

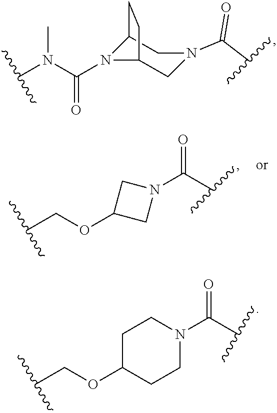 [SMILES: *C(=O)N1CC2CCC(C1)N2C(=O)N(*)C ; *COC1CN(C(*)=O)C1 ; *COC1CCN(C(*)=O)CC1]

In some embodiments, a linker of Formula II is selected from the group consisting of

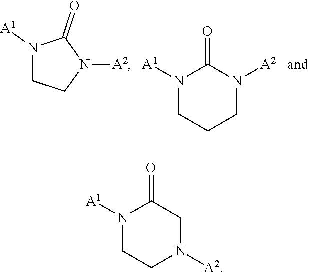

In some embodiments of compounds of the present invention, W comprises a carbodiimide. In some embodiments, W has the structure of Formula IIIa:

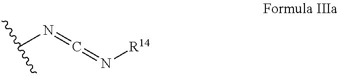

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl. In some embodiments, W has the structure:

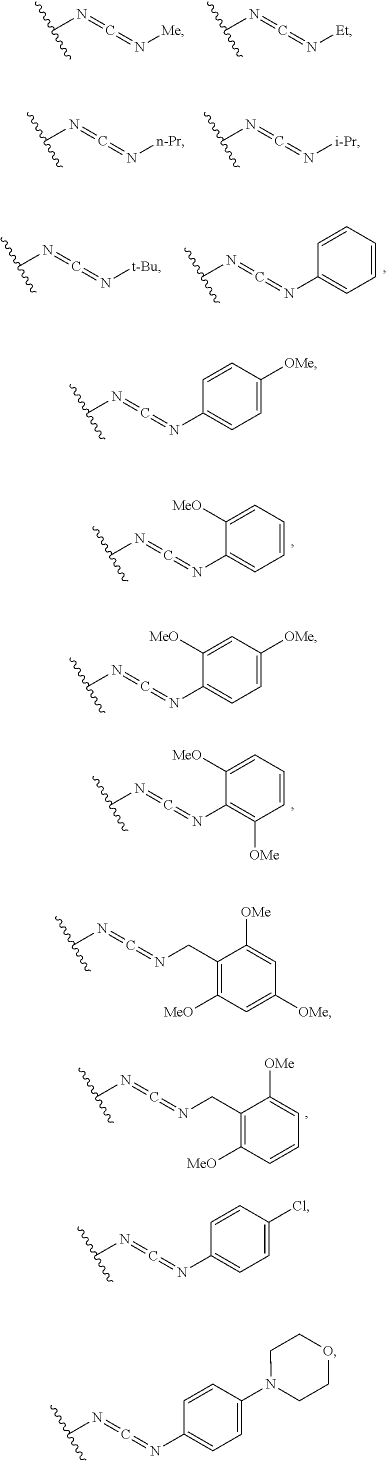

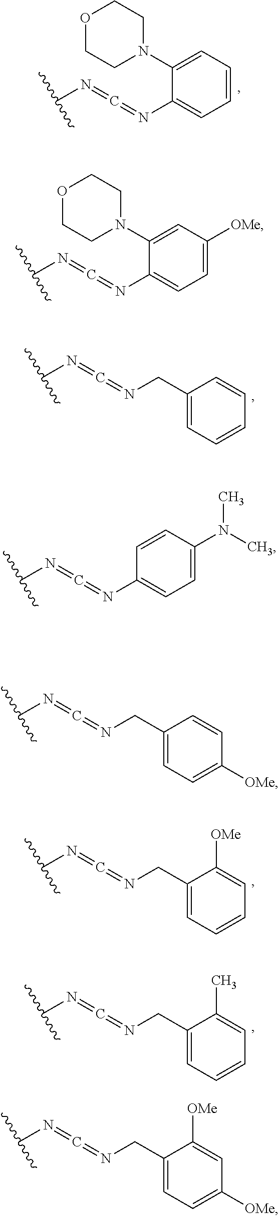

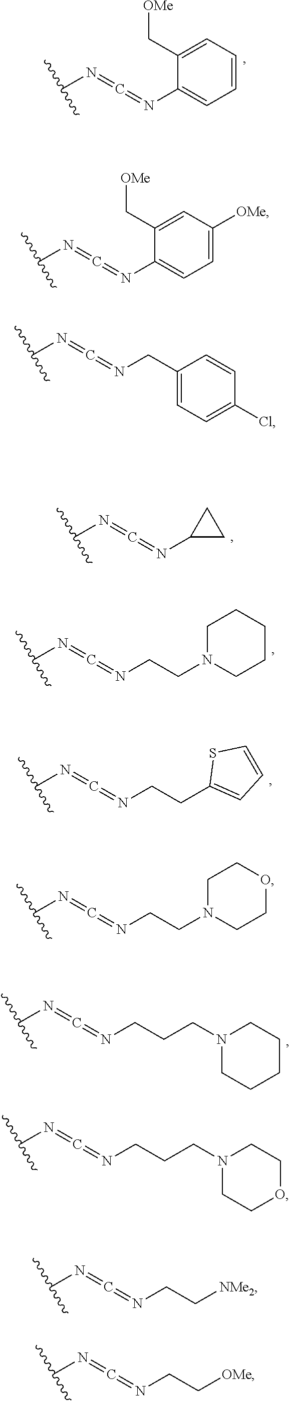

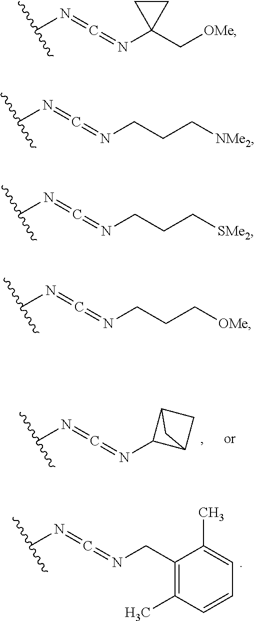

In some embodiments, W comprises an oxazoline or thiazoline. In some embodiments, W has the structure of Formula IIIb:

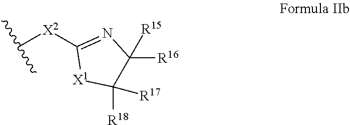

wherein X¹ is O or S;
X² is absent or NR¹⁹;
R¹⁵, R¹⁶, R¹⁷, and R¹⁸ are, independently, hydrogen or optionally substituted C₁-C₆ alkyl; and
R¹⁹ is hydrogen, C(O) (optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

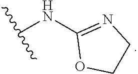 [SMILES: *NC1=NCCO1]

In some embodiments, W comprises a chloroethyl urea, a chloroethyl thiourea, a chloroethyl carbamate, or a chloroethyl thiocarbamate. In some embodiments, W has the structure of Formula IIIc:

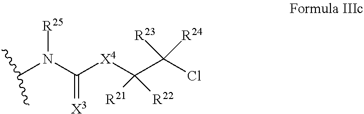

wherein X³ is O or S;
X⁴ is O, S, NR²⁶;
R²¹, R²², R²³, R²⁴, and R²⁶ are, independently, hydrogen or optionally substituted C₁-C₆ alkyl; and
R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

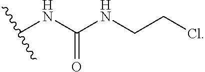 [SMILES: *NC(=O)NCCCl]

In some embodiments, W comprises an aziridine. In some embodiments, W has the structure of Formula IIId1, Formula IIld2, Formula IIld3, or Formula IIld4:

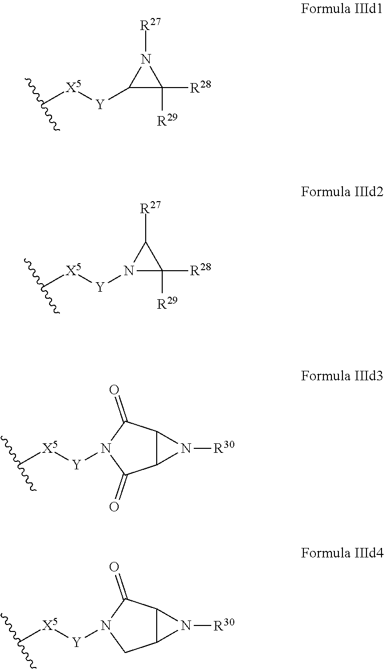

wherein X⁵ is absent or NR³⁰;
Y is absent or C(O), C(S), S (O), SO₂, or optionally substituted C₁-C₃ alkylene;
R²⁷ is hydrogen, —C(O)R³², —C(O)OR³², —SOR³³, —SO₂R³³, optionally substituted C₁-C₆ alkyl, optionally substituted 6 to 10-membered aryl, optionally substituted 3 to 14-membered heterocycloalkyl, or optionally substituted 5 to 10-membered heteroaryl;
R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;
each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;
R³⁰ is hydrogen or optionally substituted C₁-C₆ alkyl; and
R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is:

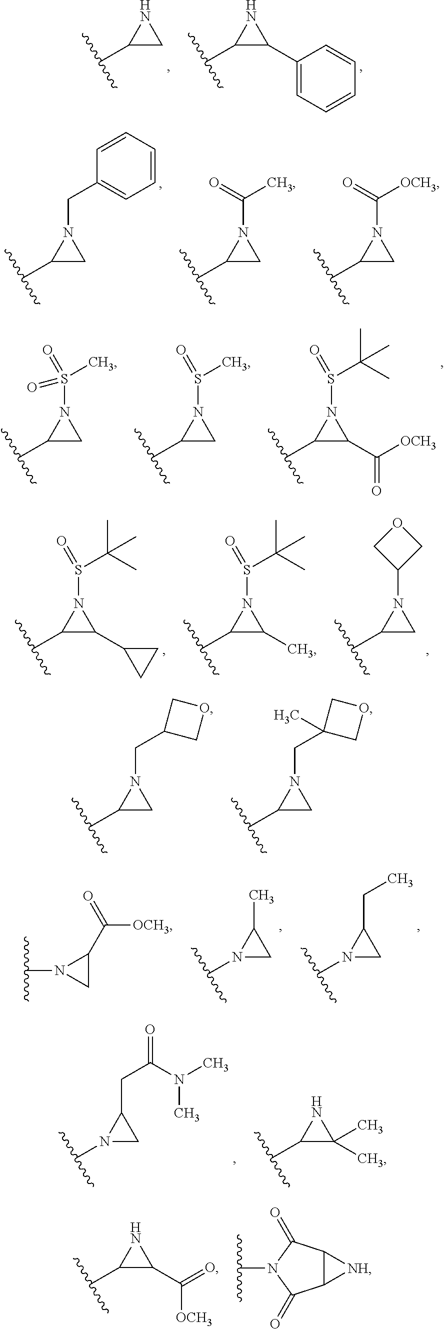

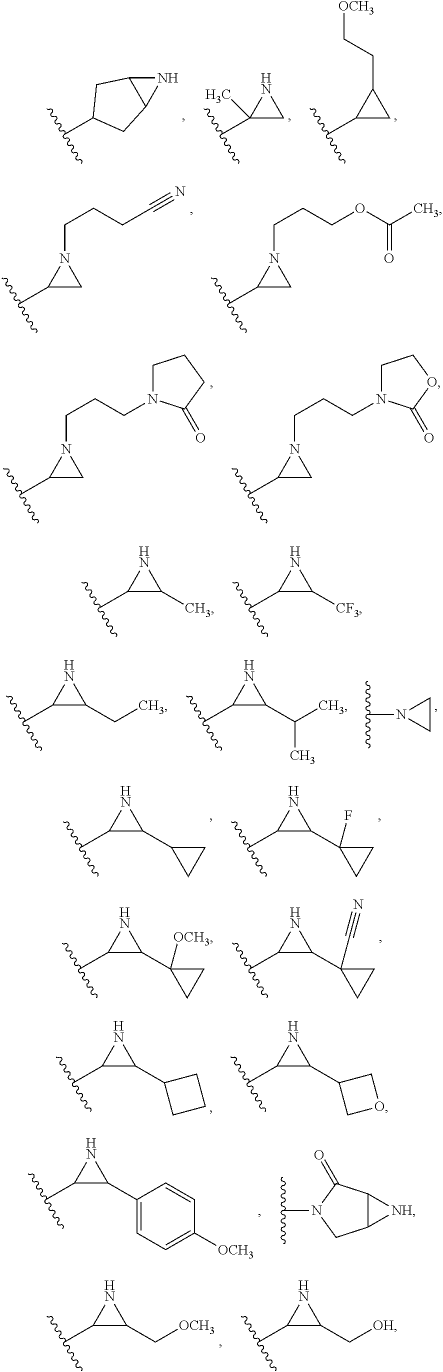

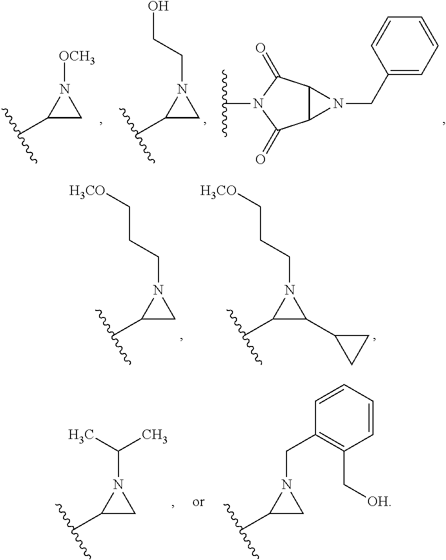

In some embodiments, W comprises an epoxide. In some embodiments, W is

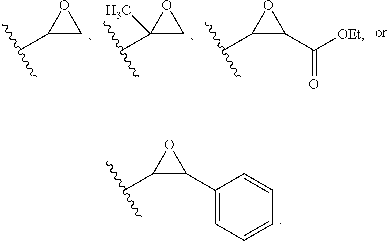

In some embodiments, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| 1 | 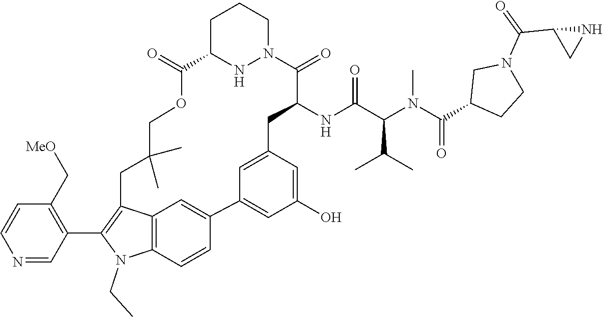 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@H]3CN3)C1)C(=O)N1CCC[C@H](N1)C(=O)OCC(C)(C)C2] |
| 2 | 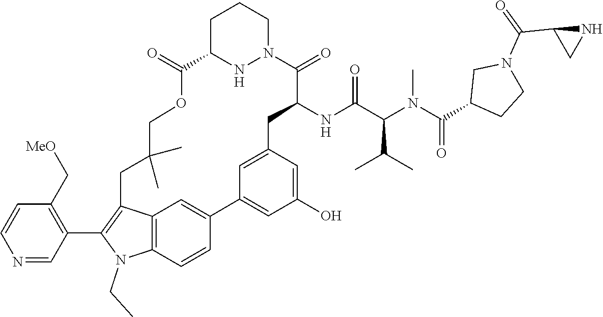 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CN3)C1)C(=O)N1CCC[C@H](N1)C(=O)OCC(C)(C)C2] |
| 3 | 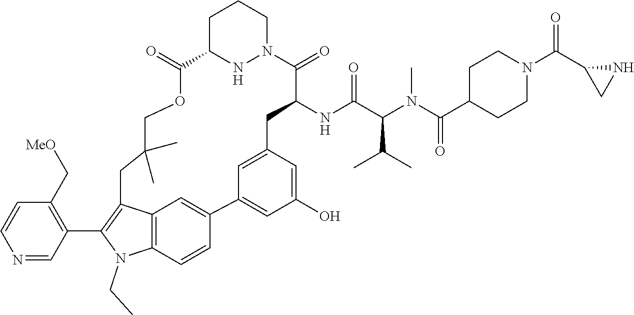 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@H]3CN3)CC1)C(=O)N1CCC[C@H](N1)C(=O)OCC(C)(C)C2] |
| 4 | 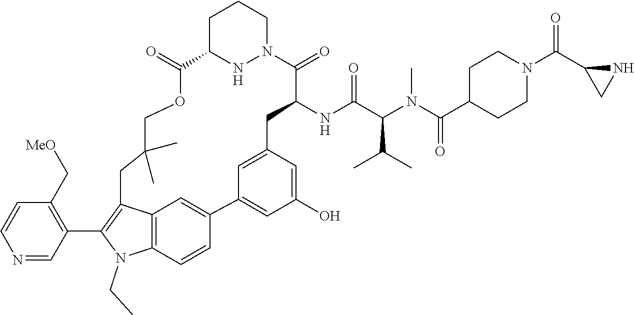 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@@H]3CN3)CC1)C(=O)N1CCC[C@H](N1)C(=O)OCC(C)(C)C2] |
| 5 | 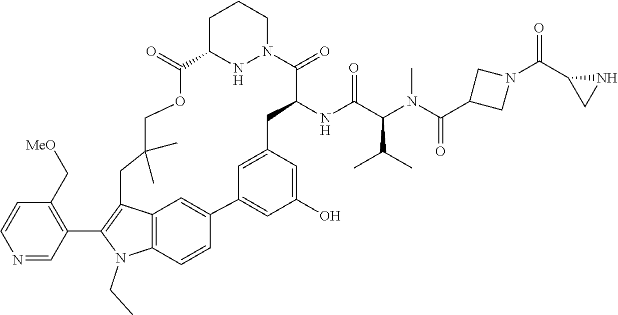 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@H]3CN3)C1)C(=O)N1CCC[C@H](N1)C(=O)OCC(C)(C)C2] |
| 6 | 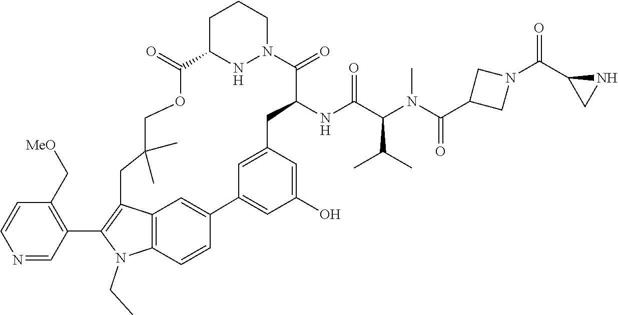 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@@H]3CN3)C1)C(=O)N1CCC[C@H](N1)C(=O)OCC(C)(C)C2] |
| 7 | 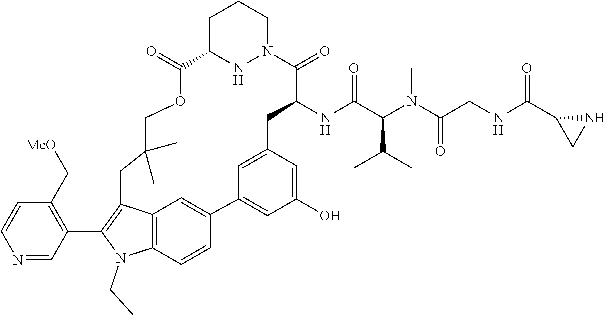 [SMILES: CCn1c(-c2cnccc2COC)c2c3cc(ccc31)-c1cc(O)cc(c1)C[C@H](NC(=O)[C@H](C(C)C)N(C)C(=O)CNC(=O)[C@H]1CN1)C(=O)N1CCC[C@H](N1)C(=O)OCC(C)(C)C2] |
| 8 | 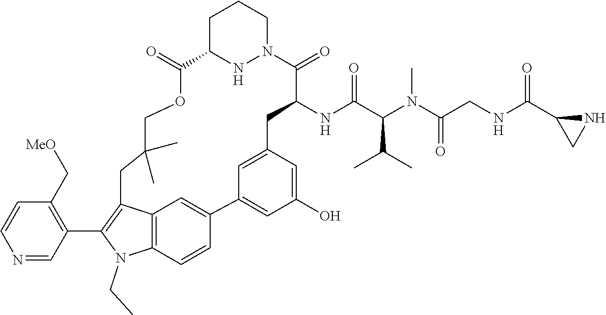 [SMILES: CCn1c(-c2cnccc2COC)c2c3cc(ccc31)-c1cc(O)cc(c1)C[C@H](NC(=O)[C@H](C(C)C)N(C)C(=O)CNC(=O)[C@@H]1CN1)C(=O)N1CCC[C@H](N1)C(=O)OCC(C)(C)C2] |
| 9 | 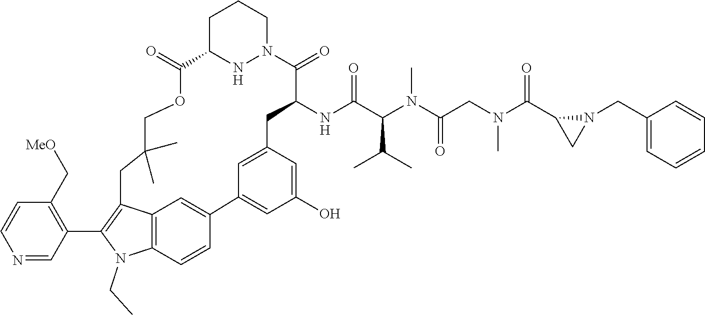 |
| 10 | 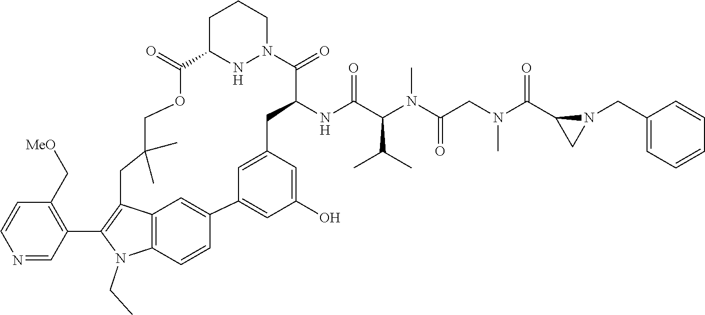 |
| 11 | 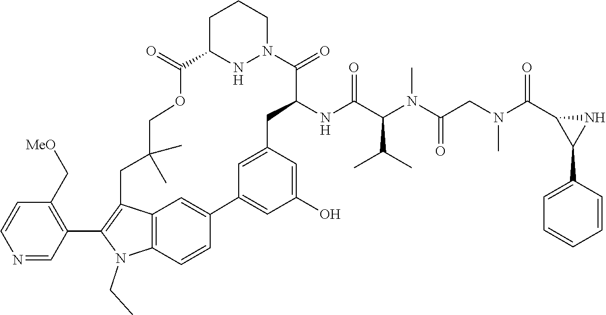 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccccc1)C(=O)N1CCC[C@H](N1)C(=O)OCC(C)(C)C2] |
| 12 | 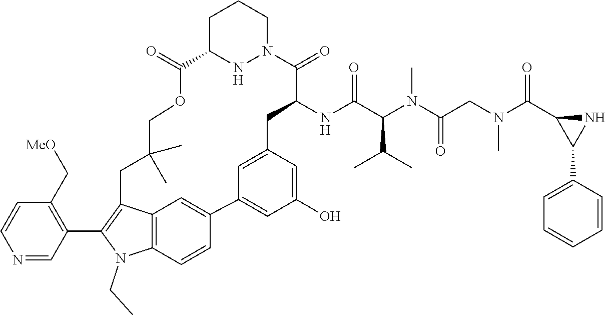 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccccc1)C(=O)N1CCC[C@H](N1)C(=O)OCC(C)(C)C2] |
| 13 | 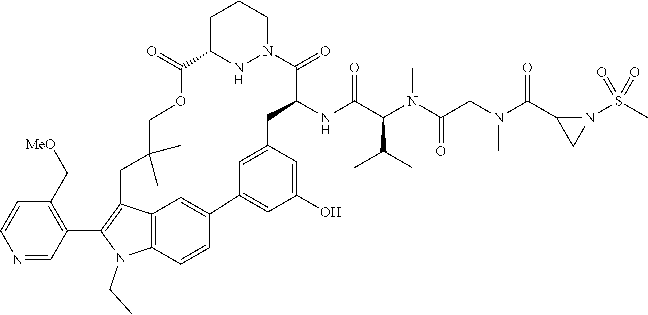 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1S(C)(=O)=O)C(=O)N1CCC[C@H](N1)C(=O)OCC(C)(C)C2] |
| 14 | 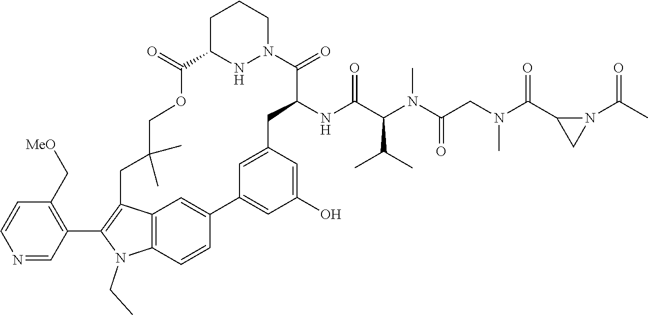 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1C(C)=O)C(=O)N1CCC[C@H](N1)C(=O)OCC(C)(C)C2] |
| 15 A and B | 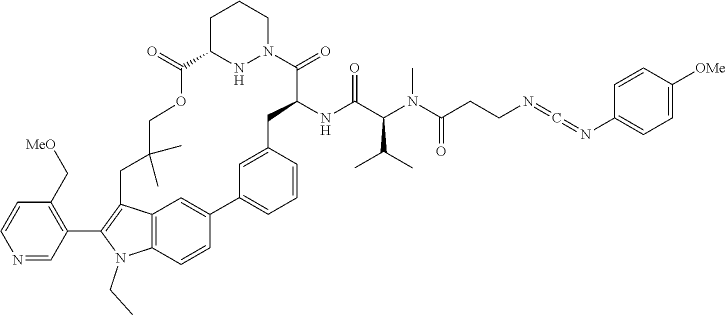 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1)C(=O)N1CCC[C@H](N1)C(=O)OCC(C)(C)C2] |
| 16 A and B | 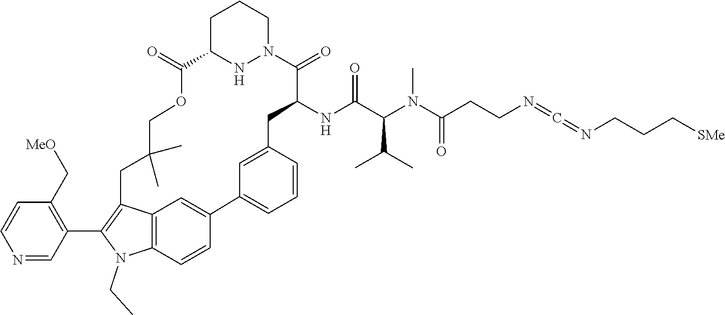 [SMILES: CCn1c(-c2cnccc2COC)c2c3cc(ccc31)-c1cccc(c1)C[C@H](NC(=O)[C@H](C(C)C)N(C)C(=O)CCN=C=NCCCSC)C(=O)N1CCC[C@H](N1)C(=O)OCC(C)(C)C2] |
| 17 A and B | 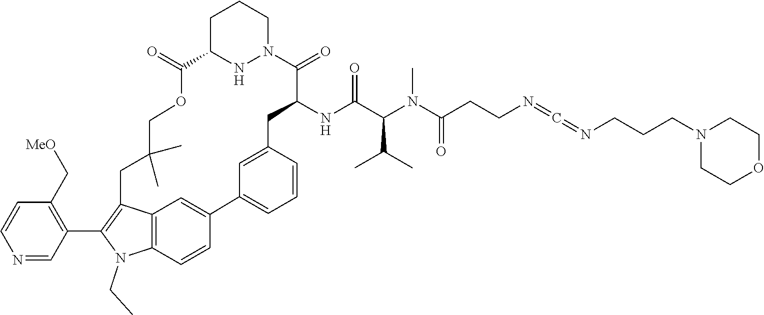 [SMILES: CCn1c(-c2cnccc2COC)c2c3cc(ccc31)-c1cccc(c1)C[C@H](NC(=O)[C@H](C(C)C)N(C)C(=O)CCN=C=NCCCN1CCOCC1)C(=O)N1CCC[C@H](N1)C(=O)OCC(C)(C)C2] |
| 18 A and B | 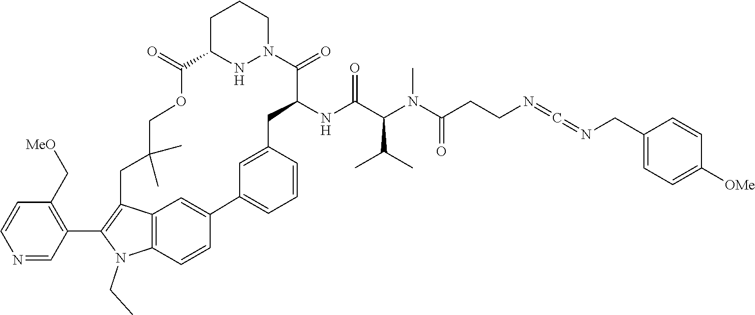 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1)C(=O)N1CCC[C@H](N1)C(=O)OCC(C)(C)C2] |
| 19 A and B | 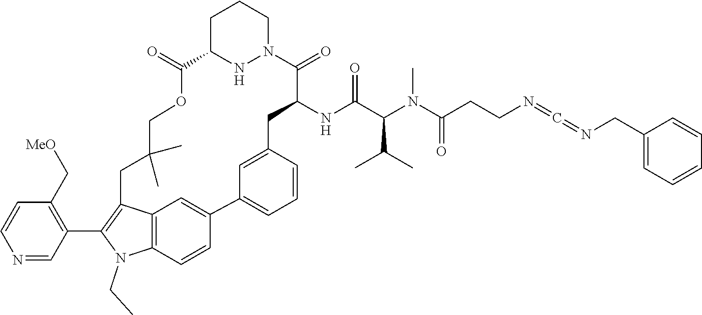 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1)C(=O)N1CCC[C@H](N1)C(=O)OCC(C)(C)C2] |
| 20 A and B | 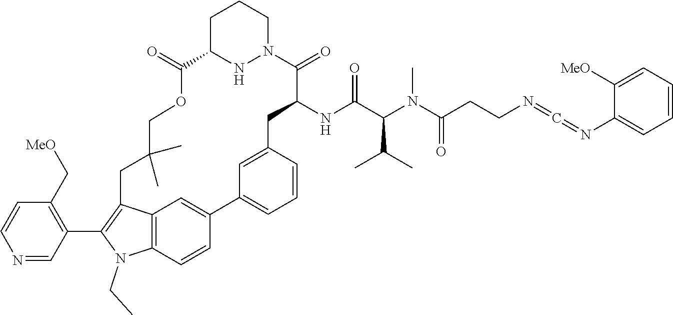 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1OC)C(=O)N1CCC[C@H](N1)C(=O)OCC(C)(C)C2] |
| 21 A and B | 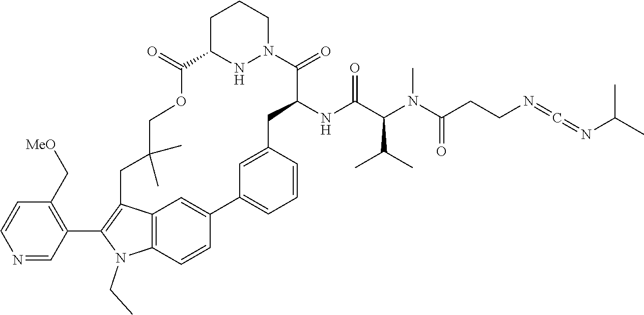 [SMILES: CCn1c(-c2cnccc2COC)c2c3cc(ccc31)-c1cccc(c1)C[C@H](NC(=O)[C@H](C(C)C)N(C)C(=O)CCN=C=NC(C)C)C(=O)N1CCC[C@H](N1)C(=O)OCC(C)(C)C2] |
| 22 A and B | 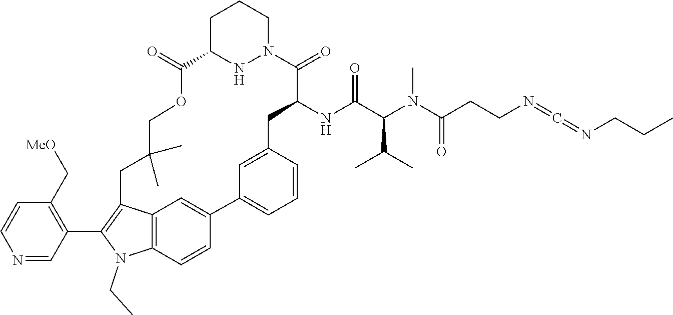 [SMILES: CCCN=C=NCCC(=O)N(C)[C@H](C(=O)N[C@H]1Cc2cccc(c2)-c2ccc3c(c2)c(c(-c2cnccc2COC)n3CC)CC(C)(C)COC(=O)[C@@H]2CCCN(N2)C1=O)C(C)C] |
| 23 A and B | 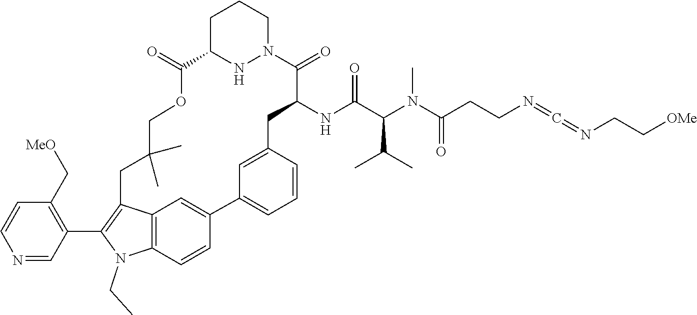 [SMILES: CCn1c(-c2cnccc2COC)c2c3cc(ccc31)-c1cccc(c1)C[C@H](NC(=O)[C@H](C(C)C)N(C)C(=O)CCN=C=NCCOC)C(=O)N1CCC[C@H](N1)C(=O)OCC(C)(C)C2] |
| 24 A and B | 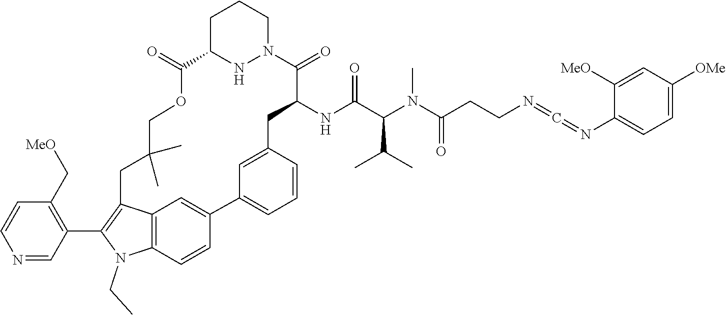 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1OC)C(=O)N1CCC[C@H](N1)C(=O)OCC(C)(C)C2] |
| 25 | 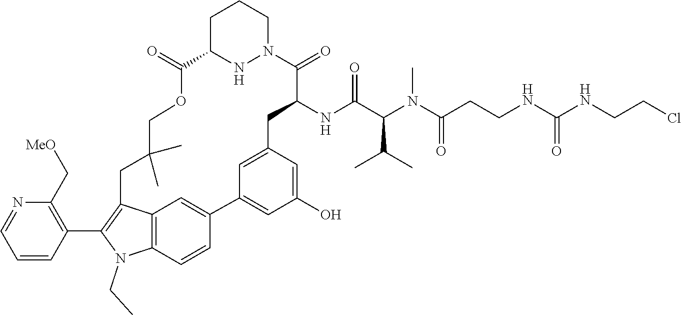 [SMILES: CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2] |
| 26 | 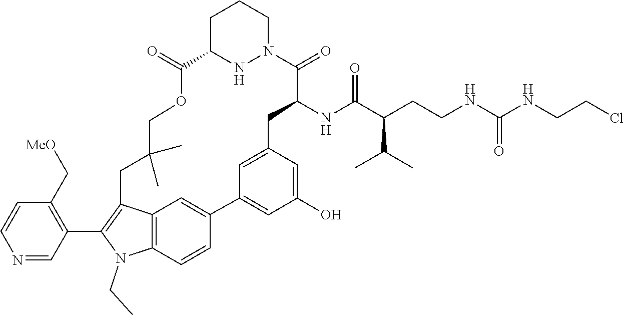 [SMILES: CCn1c(-c2cnccc2COC)c2c3cc(ccc31)-c1cc(O)cc(c1)C[C@H](NC(=O)[C@@H](CCNC(=O)NCCCl)C(C)C)C(=O)N1CCC[C@H](N1)C(=O)OCC(C)(C)C2] |
| 27 | 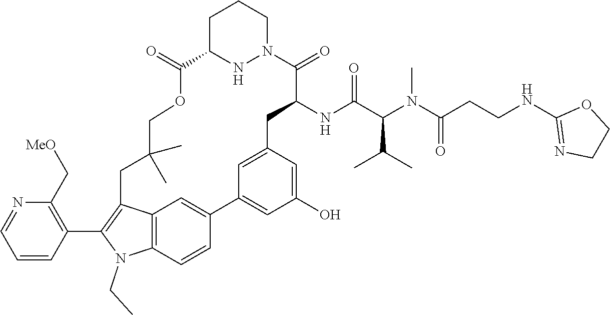 [SMILES: CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| 28 | 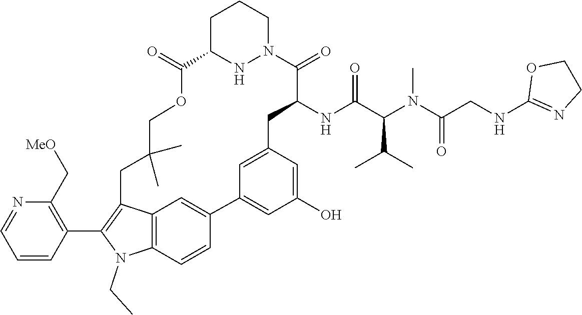 [SMILES: CCn1c(-c2cccnc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| 29 | 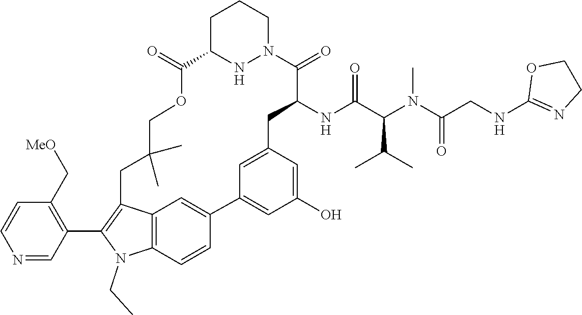 [SMILES: CCn1c(-c2cnccc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| 30 | 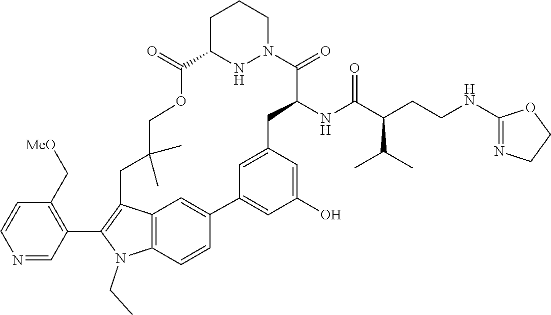 [SMILES: CCn1c(-c2cnccc2COC)c2c3cc(ccc31)-c1cc(O)cc(c1)C[C@H](NC(=O)[C@@H](CCNC1=NCCO1)C(C)C)C(=O)N1CCC[C@H](N1)C(=O)OCC(C)(C)C2] |
| 31 | 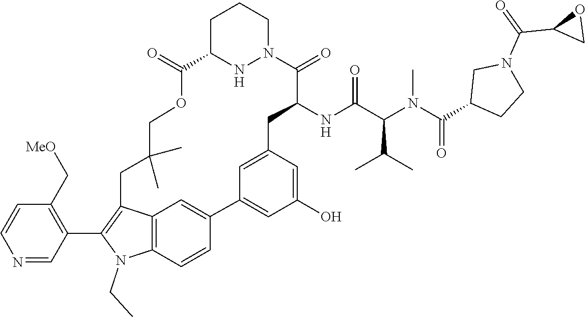 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CO3)C1)C(=O)N1CCC[C@H](N1)C(=O)OCC(C)(C)C2] |
| 32 A and B | 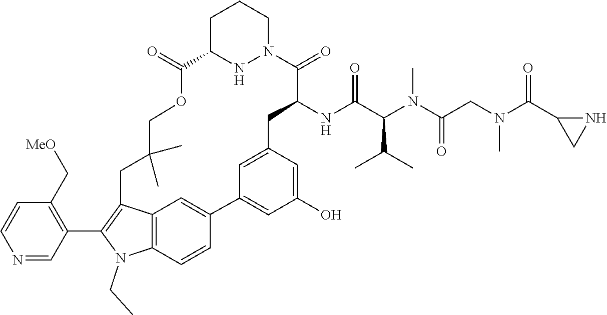 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| 33 | 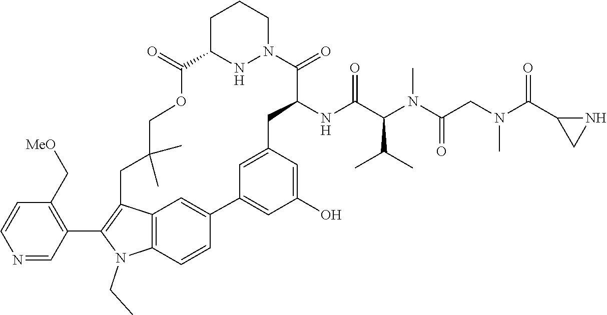 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| 34 | 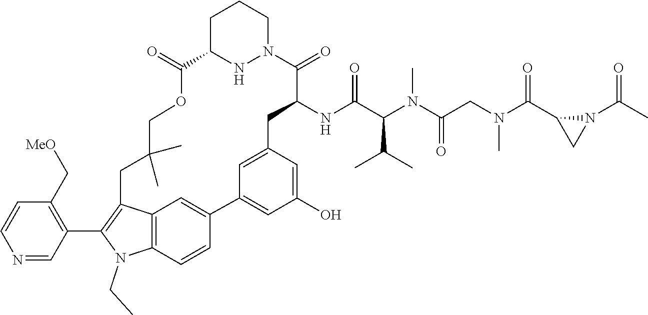 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(C)=O)C(=O)N1CCC[C@H](N1)C(=O)OCC(C)(C)C2] |
| 35 | 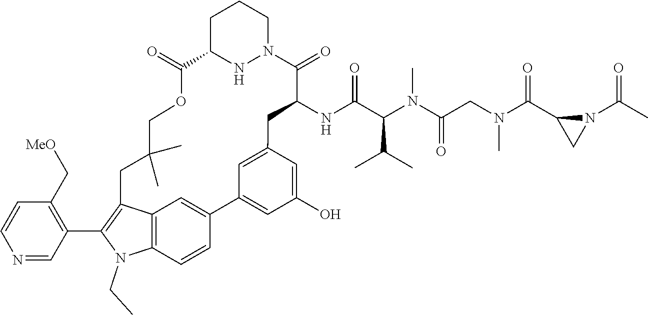 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(C)=O)C(=O)N1CCC[C@H](N1)C(=O)OCC(C)(C)C2] |
| 36 | 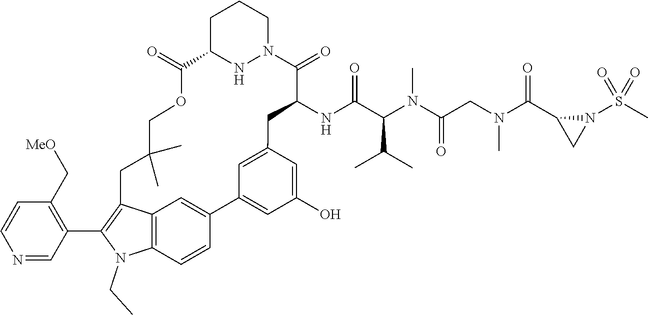 |
| 37 | 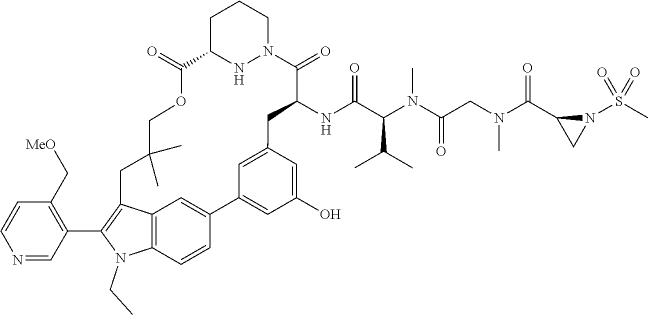 |
| 38 | 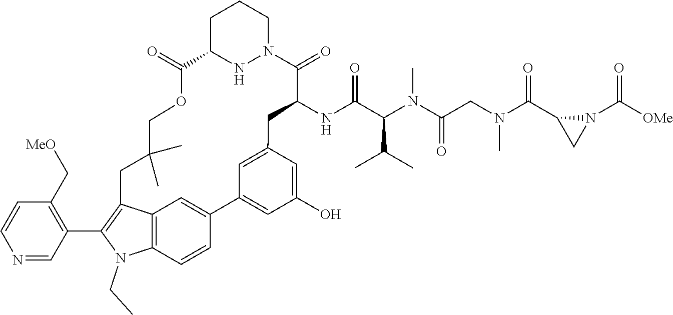 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(=O)OC)C(=O)N1CCC[C@H](N1)C(=O)OCC(C)(C)C2] |
| 39 | 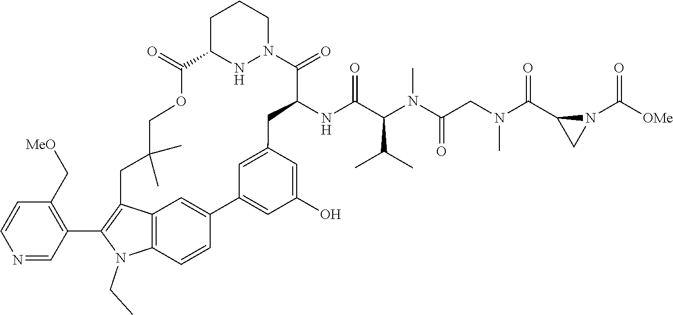 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(=O)OC)C(=O)N1CCC[C@H](N1)C(=O)OCC(C)(C)C2] |
| 40 | 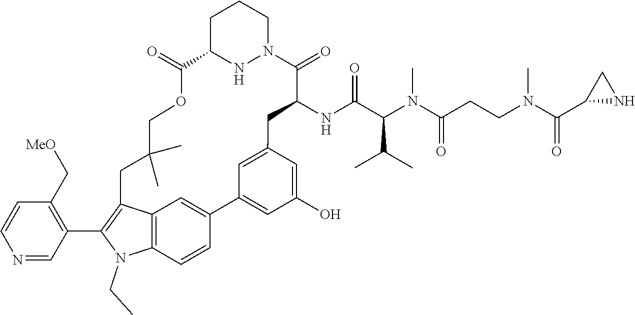 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@@H]1CN1)C(=O)N1CCC[C@H](N1)C(=O)OCC(C)(C)C2] |
| 41 | 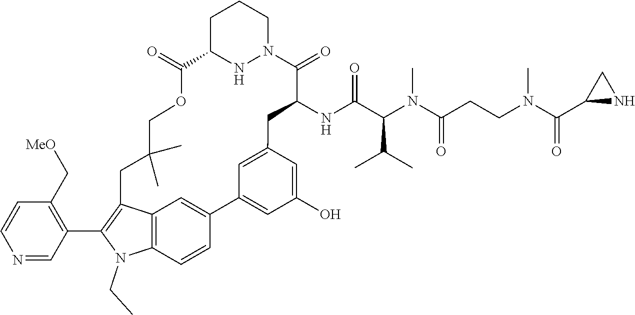 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@H]1CN1)C(=O)N1CCC[C@H](N1)C(=O)OCC(C)(C)C2] |
| 42A | 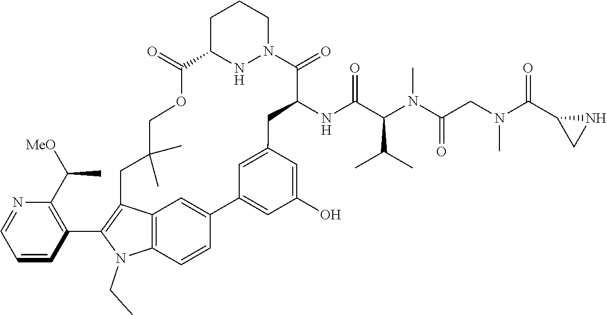 |
| 42B | 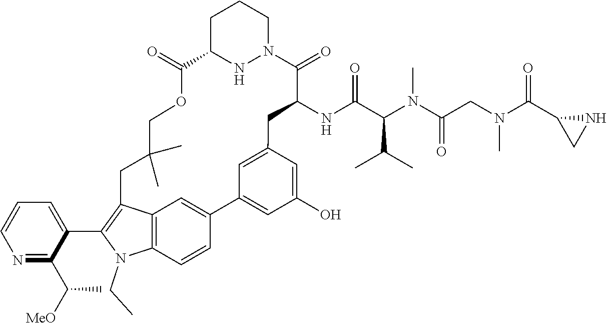 |
| 43A | 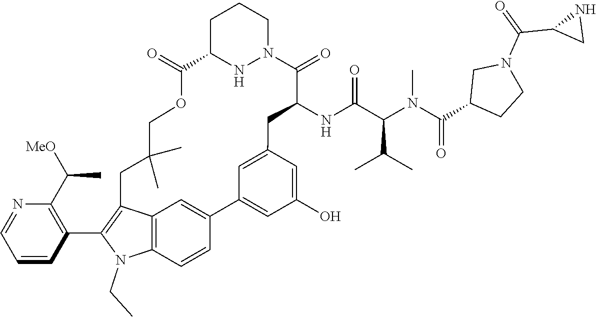 |
| 43B | 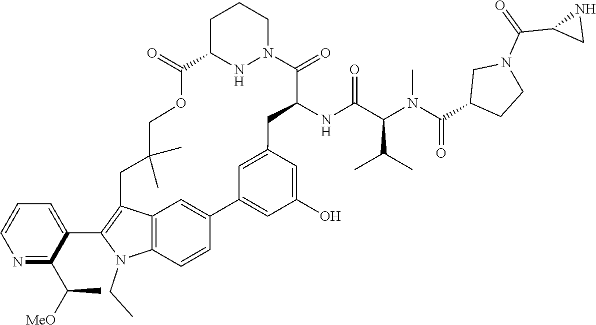 |
| 44 | 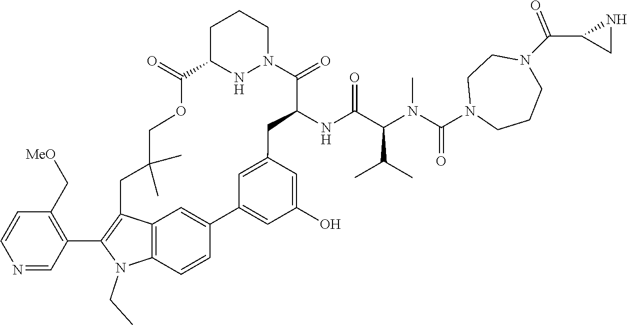 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@H]3CN3)CC1)C(=O)N1CCC[C@H](N1)C(=O)OCC(C)(C)C2] |
| 45 | 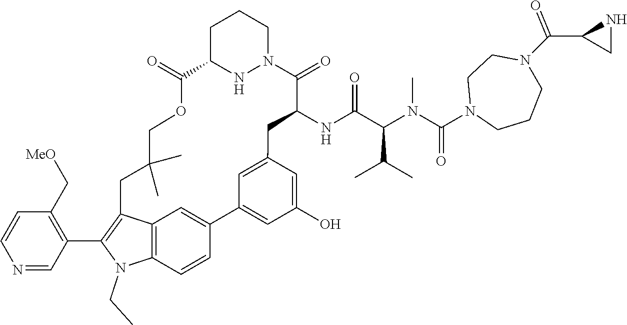 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@@H]3CN3)CC1)C(=O)N1CCC[C@H](N1)C(=O)OCC(C)(C)C2] |
| 46 | 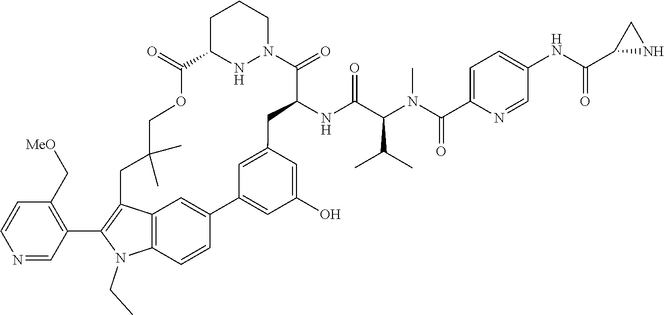 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3CN3)cn1)C(=O)N1CCC[C@H](N1)C(=O)OCC(C)(C)C2] |
| 47 A and B | 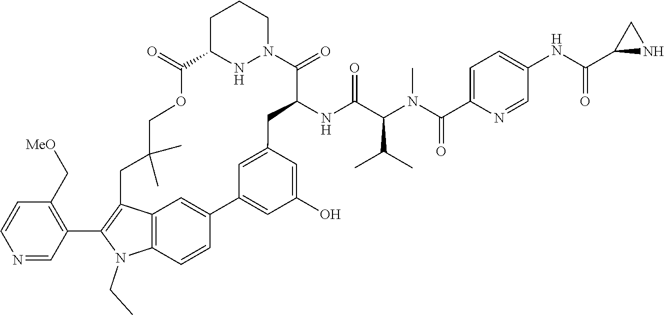 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3CN3)cn1)C(=O)N1CCC[C@H](N1)C(=O)OCC(C)(C)C2] |
| 48 | 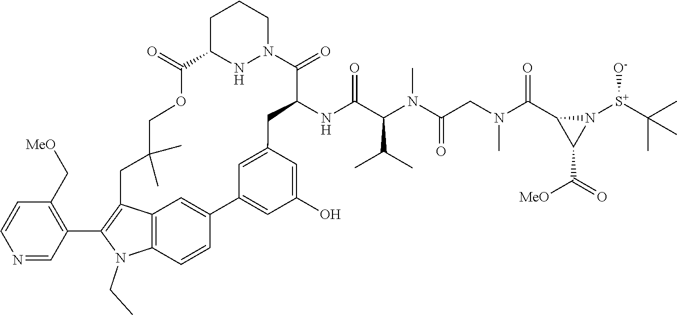 |
| 49 | 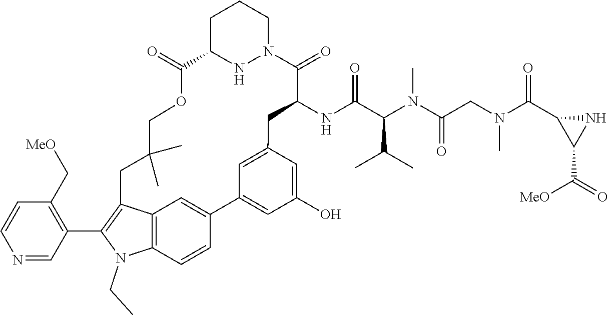 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C(=O)OC)C(=O)N1CCC[C@H](N1)C(=O)OCC(C)(C)C2] |
| 50 | 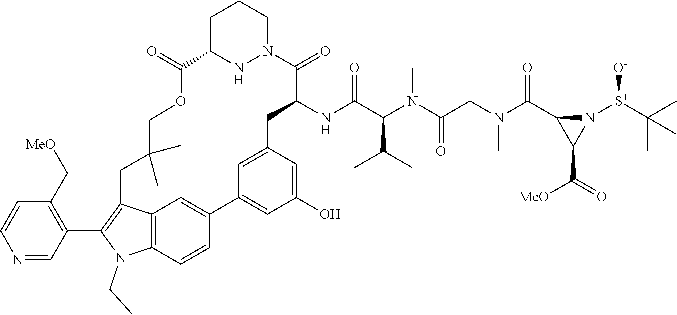 |
| 51 | 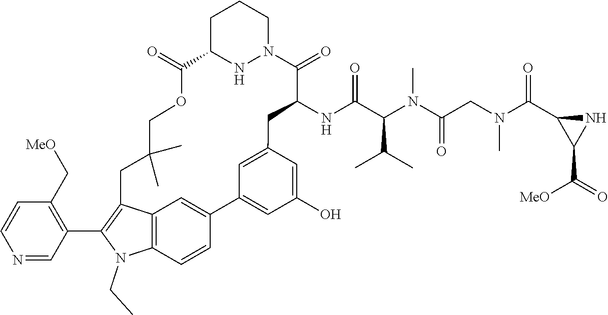 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C(=O)OC)C(=O)N1CCC[C@H](N1)C(=O)OCC(C)(C)C2] |
| 52 | 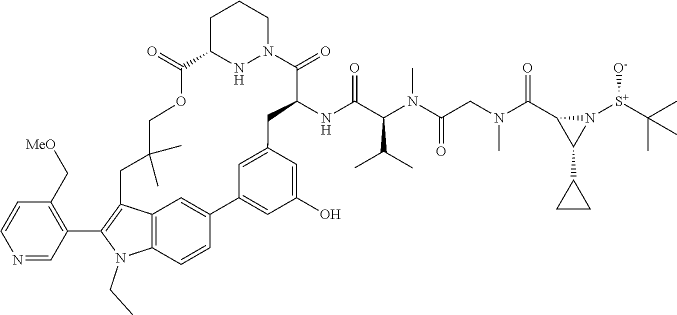 |
| 53 | 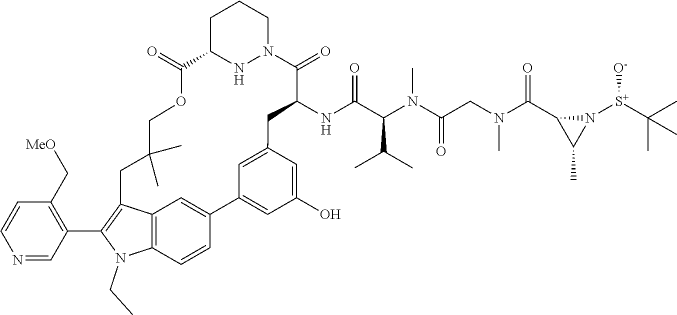 |
| 54 | 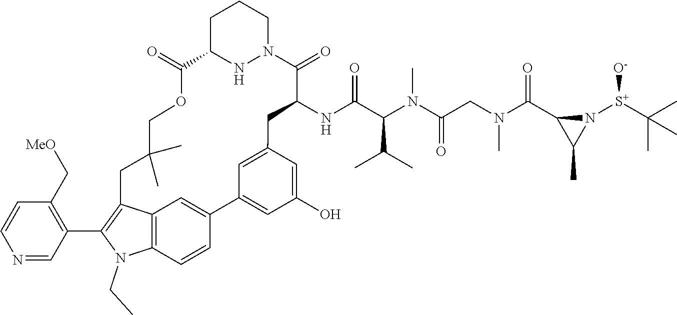 |
| 55 | 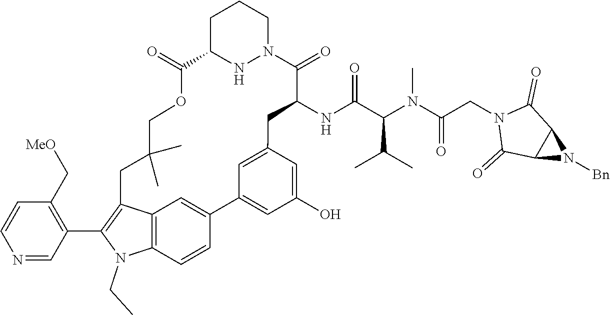 |
| 56* | 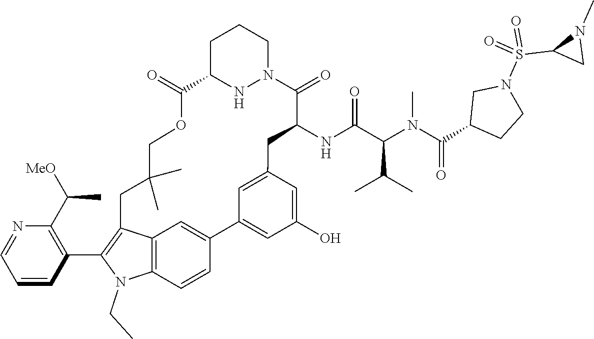 |
| 57* | 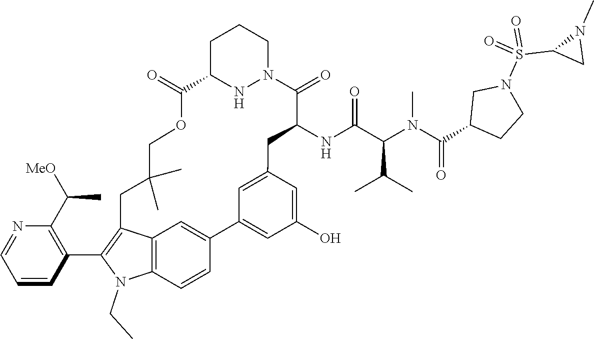 |
| 58 | 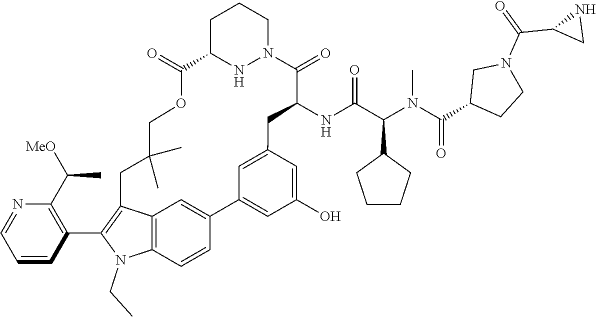 |
| 59 | 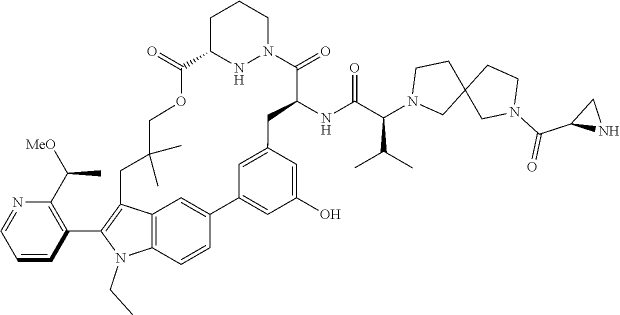 |
| 60 | 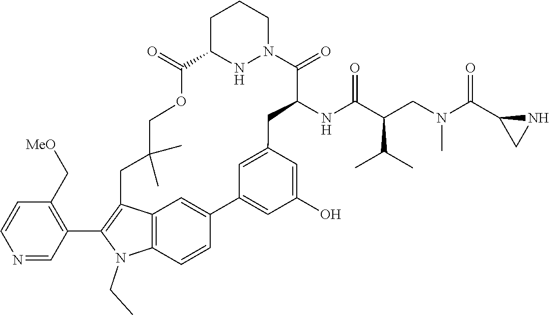 [SMILES: CCn1c(-c2cnccc2COC)c2c3cc(ccc31)-c1cc(O)cc(c1)C[C@H](NC(=O)[C@@H](CN(C)C(=O)[C@@H]1CN1)C(C)C)C(=O)N1CCC[C@H](N1)C(=O)OCC(C)(C)C2] |
| 61 A and B | 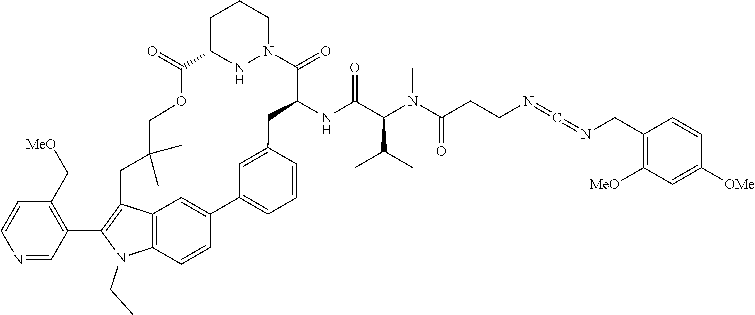 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1OC)C(=O)N1CCC[C@H](N1)C(=O)OCC(C)(C)C2] |
| 62 A and B | 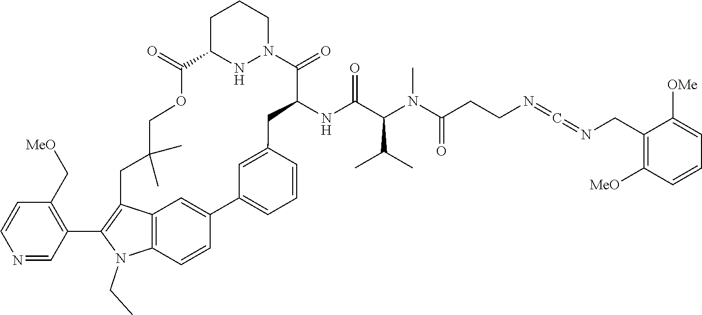 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| 63 | 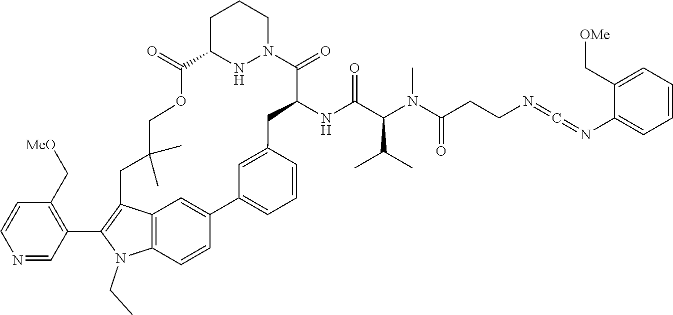 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1COC)C(=O)N1CCC[C@H](N1)C(=O)OCC(C)(C)C2] |
| 64 A and B | 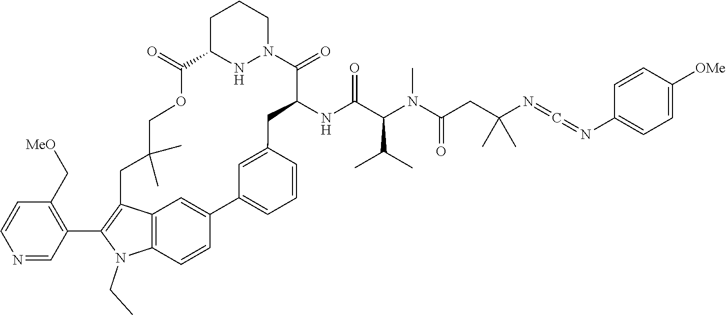 [SMILES: CCn1c(-c2cnccc2COC)c2c3cc(ccc31)-c1cccc(c1)C[C@H](NC(=O)[C@H](C(C)C)N(C)C(=O)CC(C)(C)N=C=Nc1ccc(OC)cc1)C(=O)N1CCC[C@H](N1)C(=O)OCC(C)(C)C2] |
| 65 A and B | 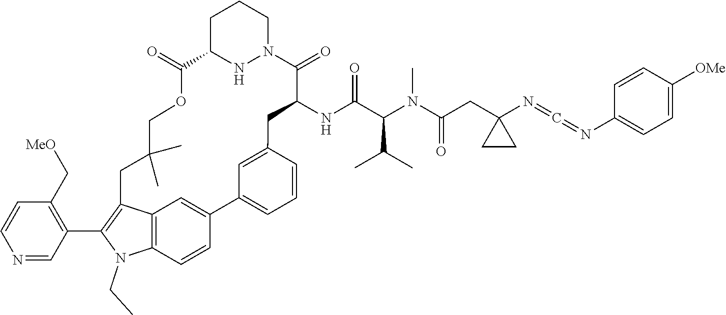 [SMILES: CCn1c(-c2cnccc2COC)c2c3cc(ccc31)-c1cccc(c1)C[C@H](NC(=O)[C@H](C(C)C)N(C)C(=O)CC1(N=C=Nc3ccc(OC)cc3)CC1)C(=O)N1CCC[C@H](N1)C(=O)OCC(C)(C)C2] |
| 66 | 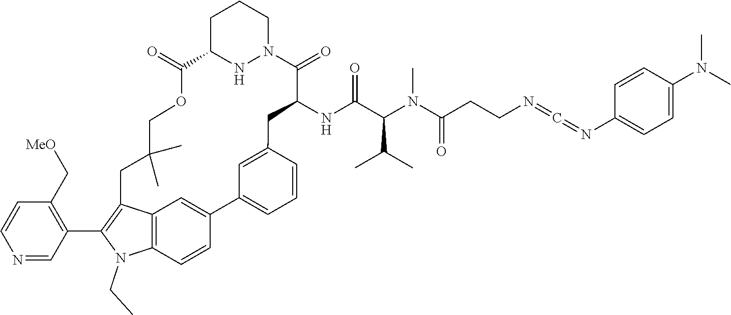 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N(C)C)cc1)C(=O)N1CCC[C@H](N1)C(=O)OCC(C)(C)C2] |
| 67 | 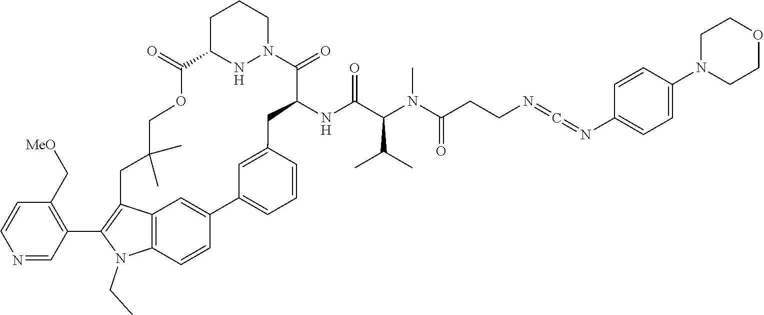 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N3CCOCC3)cc1)C(=O)N1CCC[C@H](N1)C(=O)OCC(C)(C)C2] |
| 68 A and B | 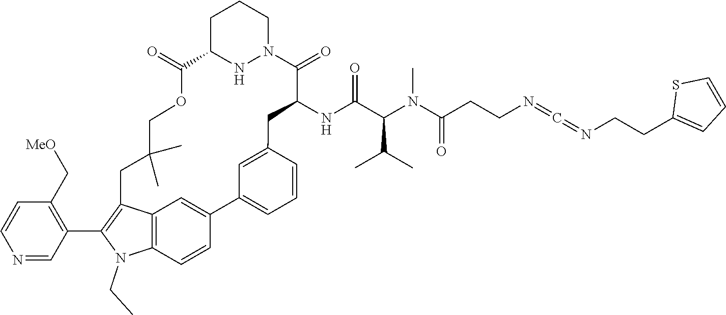 [SMILES: CCn1c(-c2cnccc2COC)c2c3cc(ccc31)-c1cccc(c1)C[C@H](NC(=O)[C@H](C(C)C)N(C)C(=O)CCN=C=NCCc1cccs1)C(=O)N1CCC[C@H](N1)C(=O)OCC(C)(C)C2] |
| 69 A and B | 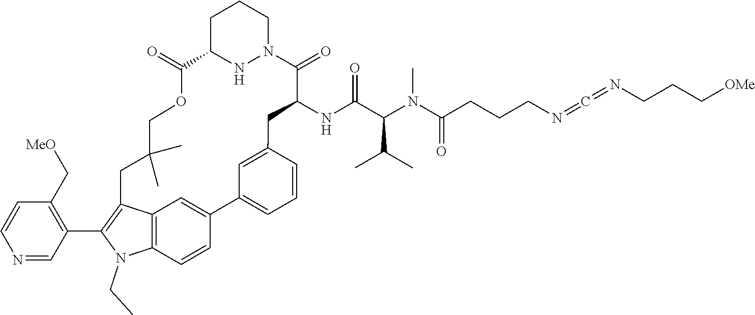 [SMILES: CCn1c(-c2cnccc2COC)c2c3cc(ccc31)-c1cccc(c1)C[C@H](NC(=O)[C@H](C(C)C)N(C)C(=O)CCCN=C=NCCCOC)C(=O)N1CCC[C@H](N1)C(=O)OCC(C)(C)C2] |
| 70 A and B | 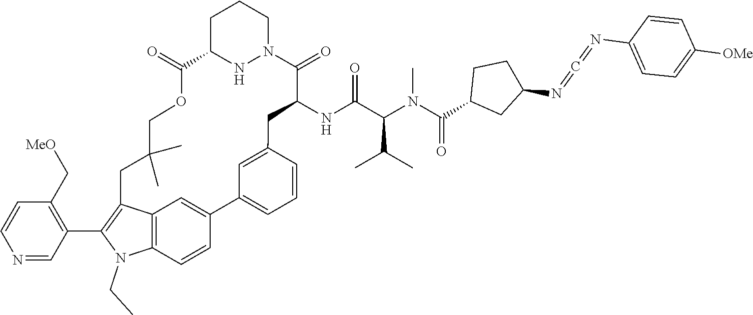 [SMILES: CCn1c(-c2cnccc2COC)c2c3cc(ccc31)-c1cccc(c1)C[C@H](NC(=O)[C@H](C(C)C)N(C)C(=O)[C@@H]1CC[C@@H](N=C=Nc3ccc(OC)cc3)C1)C(=O)N1CCC[C@H](N1)C(=O)OCC(C)(C)C2] |
| 71 A and B | 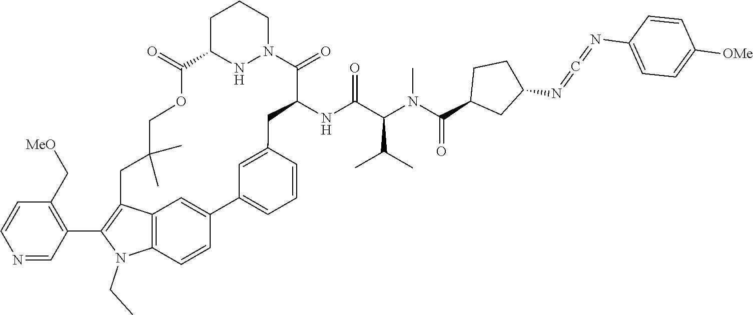 [SMILES: CCn1c(-c2cnccc2COC)c2c3cc(ccc31)-c1cccc(c1)C[C@H](NC(=O)[C@H](C(C)C)N(C)C(=O)[C@H]1CC[C@H](N=C=Nc3ccc(OC)cc3)C1)C(=O)N1CCC[C@H](N1)C(=O)OCC(C)(C)C2] |
| 72 A and B | 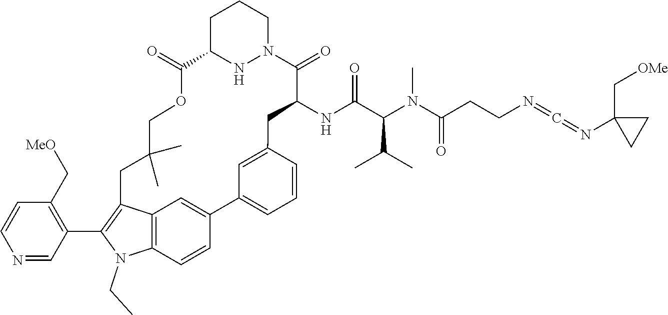 [SMILES: CCn1c(-c2cnccc2COC)c2c3cc(ccc31)-c1cccc(c1)C[C@H](NC(=O)[C@H](C(C)C)N(C)C(=O)CCN=C=NC1(COC)CC1)C(=O)N1CCC[C@H](N1)C(=O)OCC(C)(C)C2] |
| 73 | 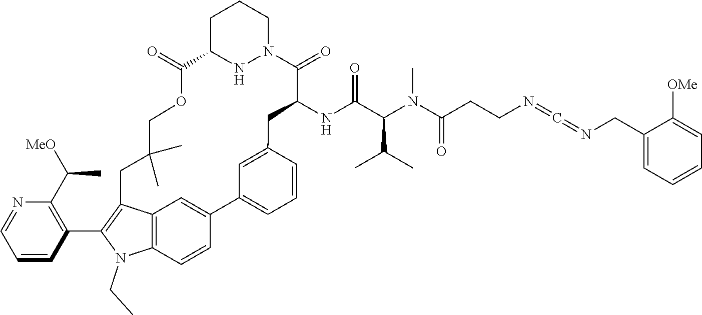 |
| 74 | 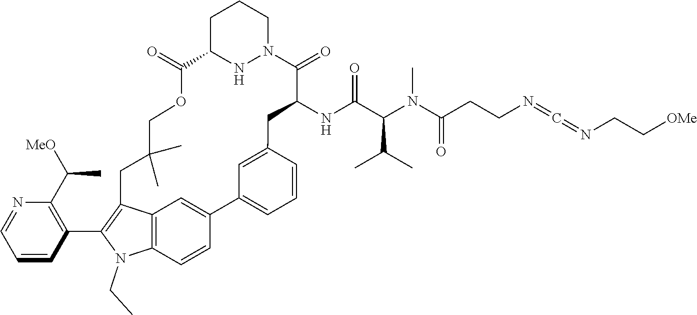 |
| 75 | 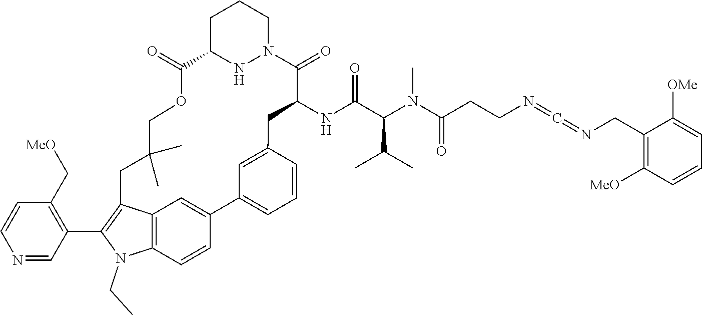 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| 76 | 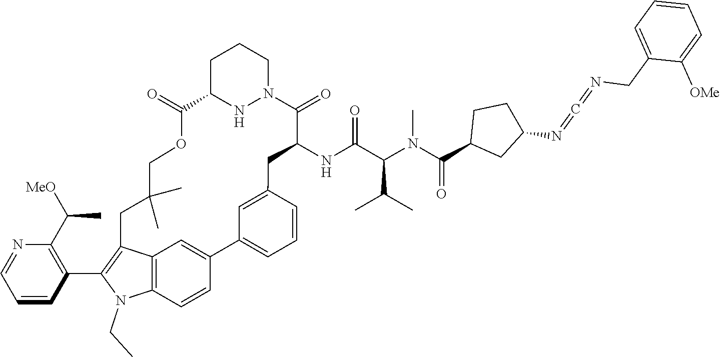 |
| 77 | 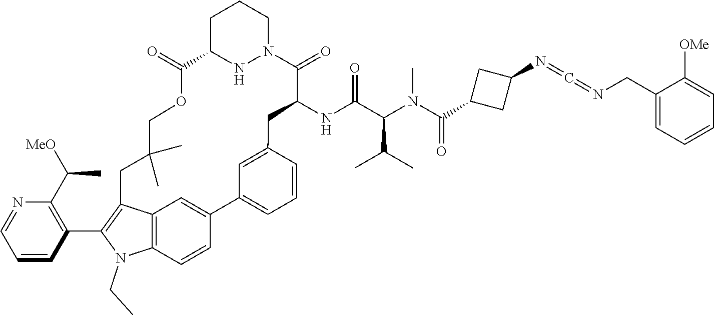 |
| 78 | 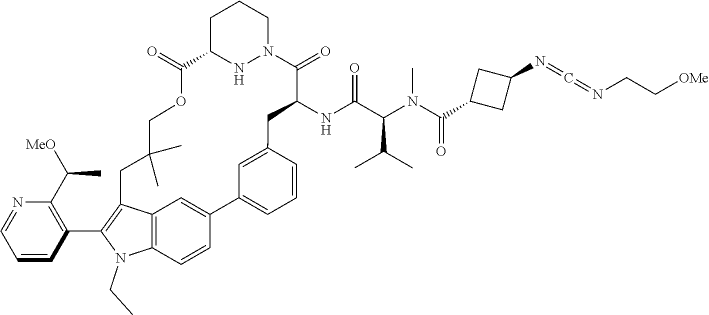 |
| 79 A and B | 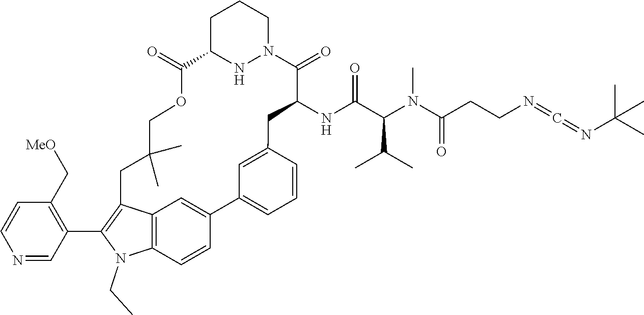 [SMILES: CCn1c(-c2cnccc2COC)c2c3cc(ccc31)-c1cccc(c1)C[C@H](NC(=O)[C@H](C(C)C)N(C)C(=O)CCN=C=NC(C)(C)C)C(=O)N1CCC[C@H](N1)C(=O)OCC(C)(C)C2] |
| 80 | 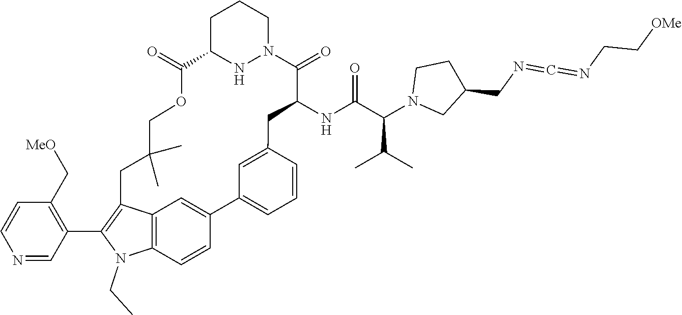 [SMILES: CCn1c(-c2cnccc2COC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| 81 | 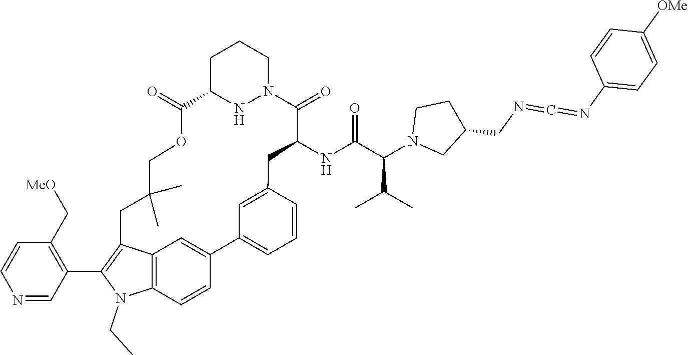 [SMILES: CCn1c(-c2cnccc2COC)c2c3cc(ccc31)-c1cccc(c1)C[C@H](NC(=O)[C@H](C(C)C)N1CC[C@H](CN=C=Nc3ccc(OC)cc3)C1)C(=O)N1CCC[C@H](N1)C(=O)OCC(C)(C)C2] |
| 82 A and B | 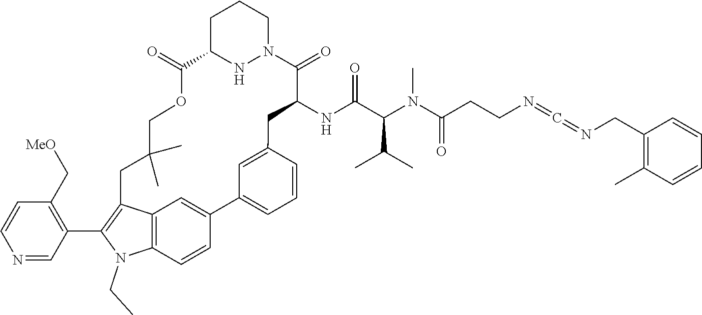 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1C)C(=O)N1CCC[C@H](N1)C(=O)OCC(C)(C)C2] |
| 83 | 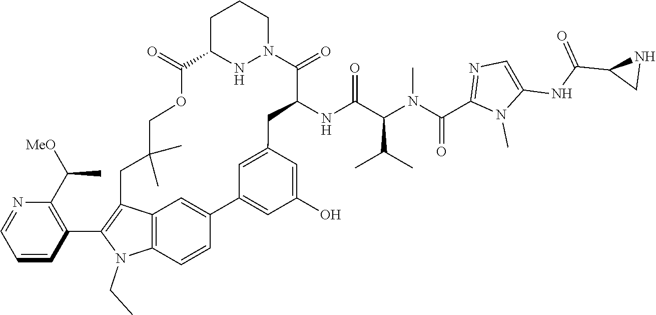 |
| 84 | 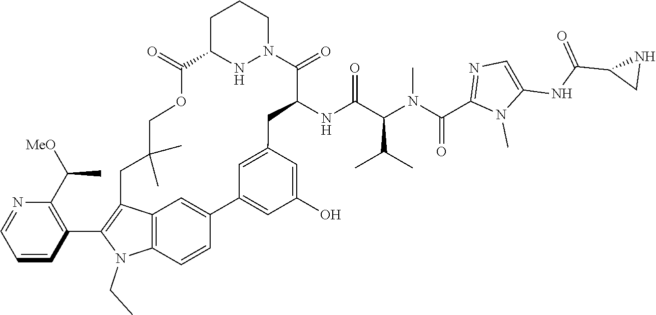 |
| 85 | 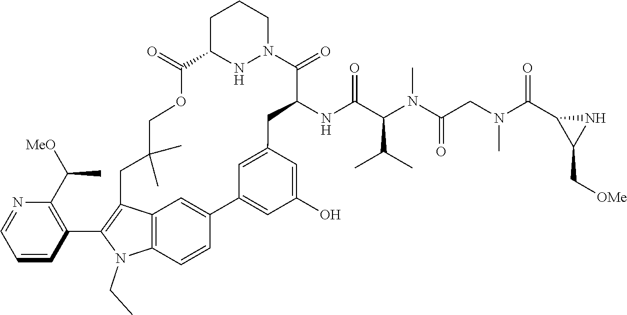 |
| 86 | 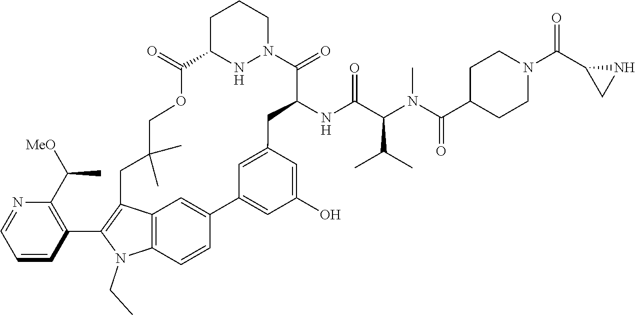 |
| 87 | 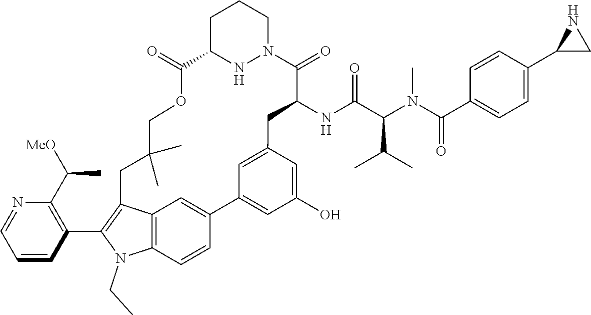 |
| 88 | 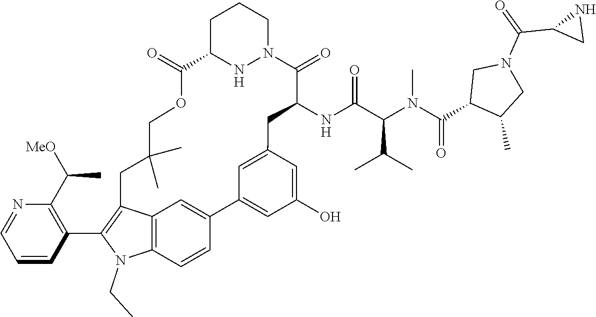 |
| 89 | 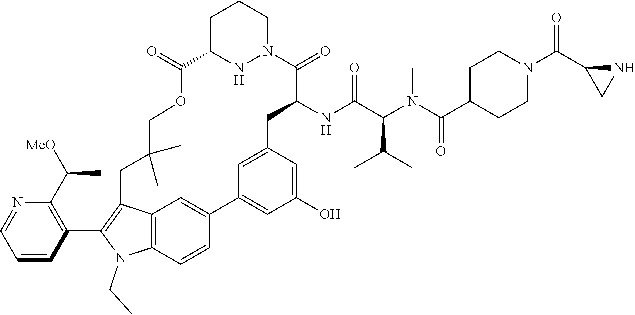 |
| 90 | 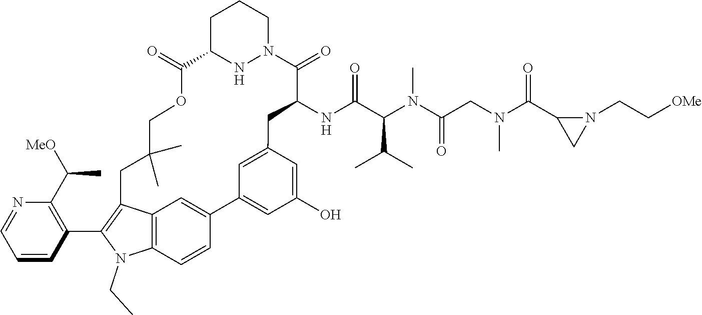 |
| 91* | 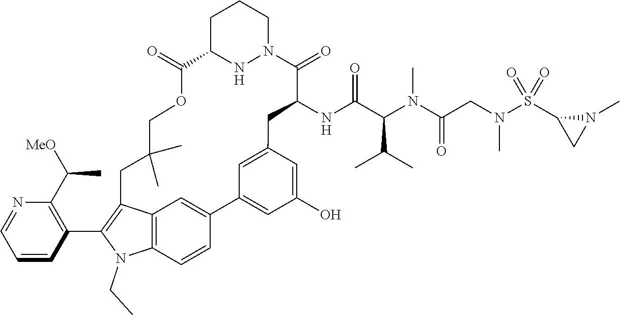 |
| 92* | 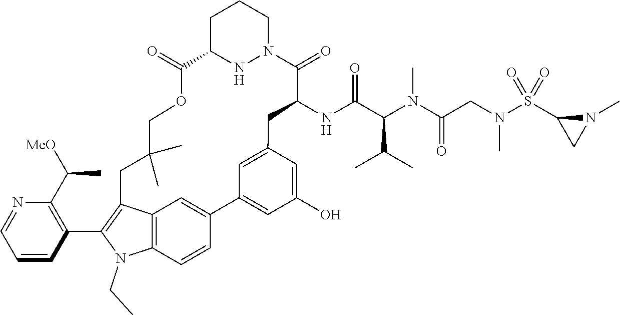 |
| 93 | 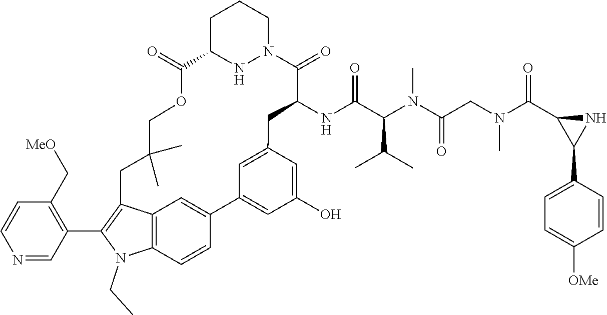 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccc(OC)cc1)C(=O)N1CCC[C@H](N1)C(=O)OCC(C)(C)C2] |
| 94 | 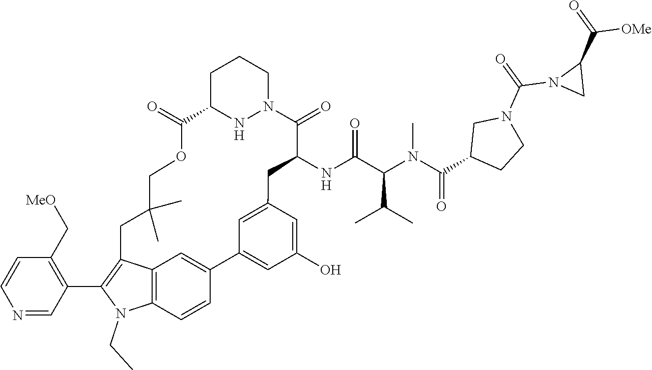 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@@H]3C(=O)OC)C1)C(=O)N1CCC[C@H](N1)C(=O)OCC(C)(C)C2] |
| 95 | 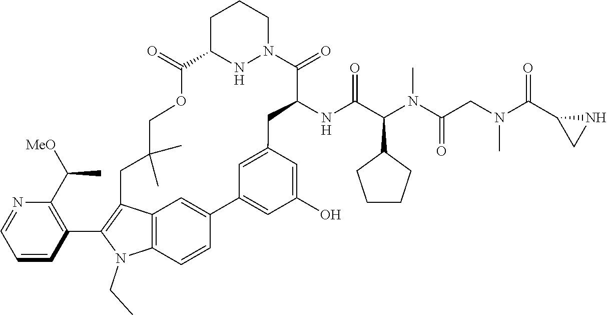 |
| 96 | 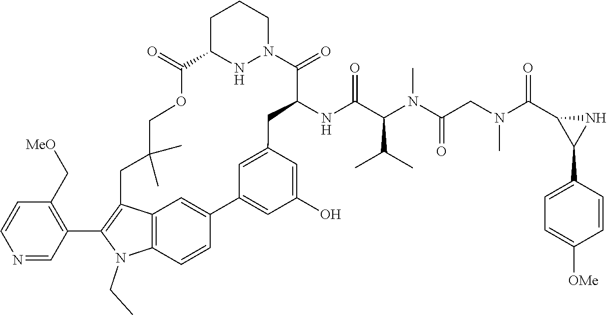 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccc(OC)cc1)C(=O)N1CCC[C@H](N1)C(=O)OCC(C)(C)C2] |
| 97 | 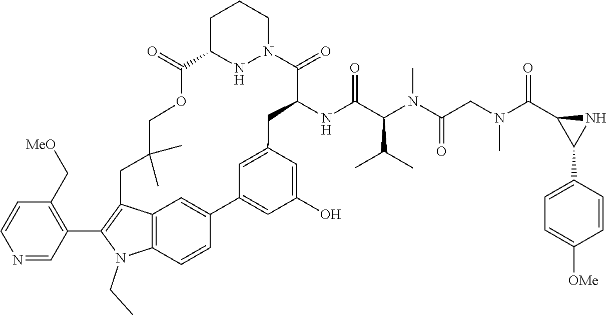 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccc(OC)cc1)C(=O)N1CCC[C@H](N1)C(=O)OCC(C)(C)C2] |
| 98 | 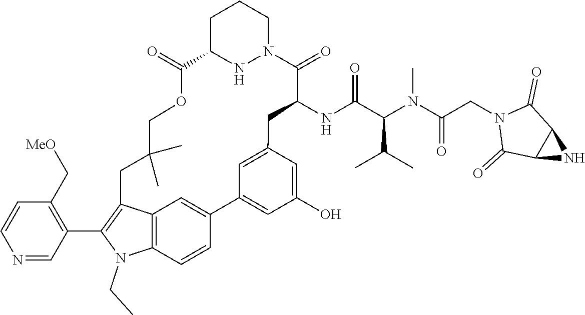 [SMILES: CCn1c(-c2cnccc2COC)c2c3cc(ccc31)-c1cc(O)cc(c1)C[C@H](NC(=O)[C@H](C(C)C)N(C)C(=O)CN1C(=O)[C@H]3N[C@H]3C1=O)C(=O)N1CCC[C@H](N1)C(=O)OCC(C)(C)C2] |
| 99 | 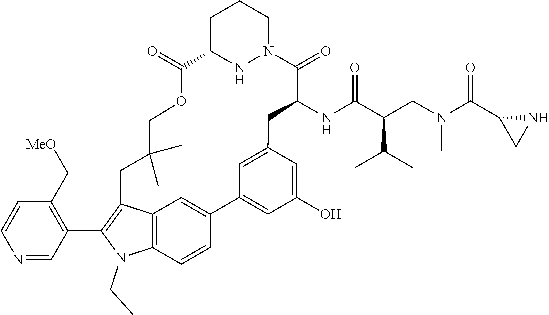 [SMILES: CCn1c(-c2cnccc2COC)c2c3cc(ccc31)-c1cc(O)cc(c1)C[C@H](NC(=O)[C@@H](CN(C)C(=O)[C@H]1CN1)C(C)C)C(=O)N1CCC[C@H](N1)C(=O)OCC(C)(C)C2] |
| 100 | 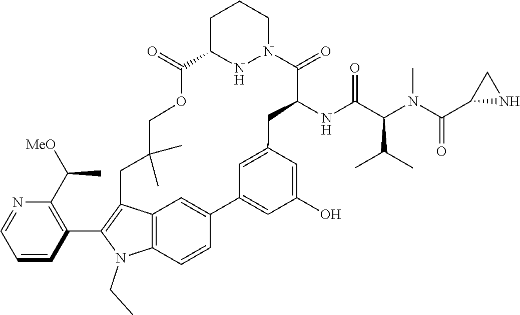 |
| 101 | 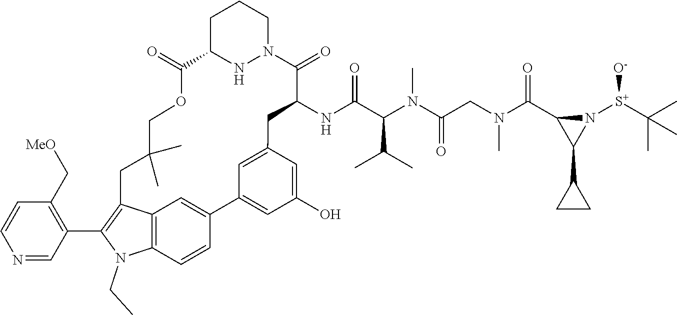 |
| 102 | 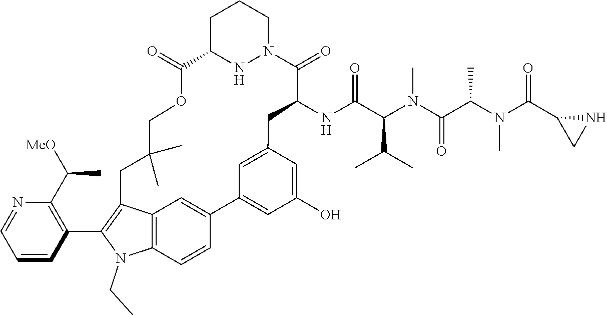 |
| 103 | 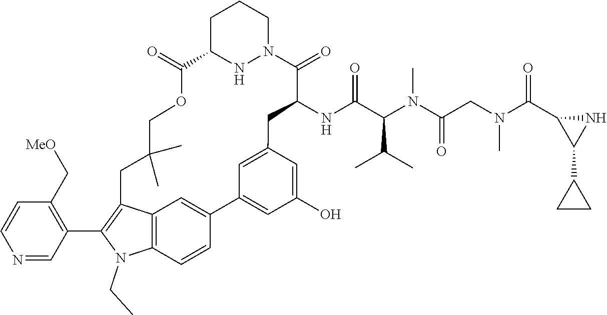 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C1CC1)C(=O)N1CCC[C@H](N1)C(=O)OCC(C)(C)C2] |
| 104 | 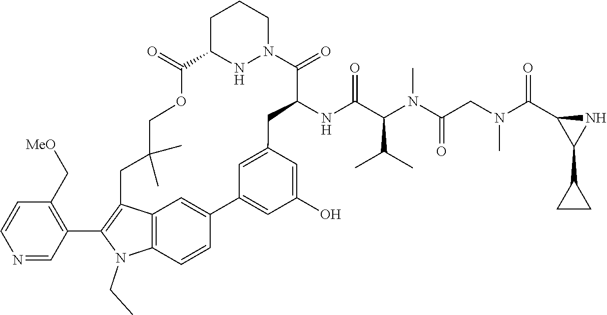 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C1CC1)C(=O)N1CCC[C@H](N1)C(=O)OCC(C)(C)C2] |
| 105 | 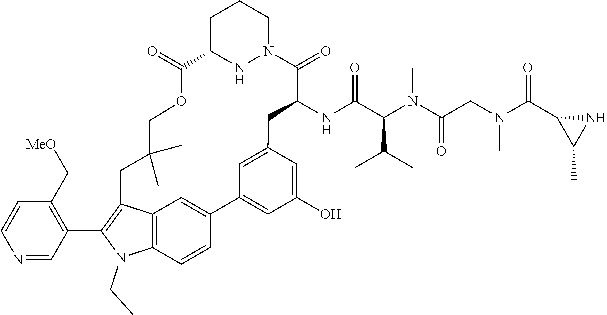 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C)C(=O)N1CCC[C@H](N1)C(=O)OCC(C)(C)C2] |
| 106 | 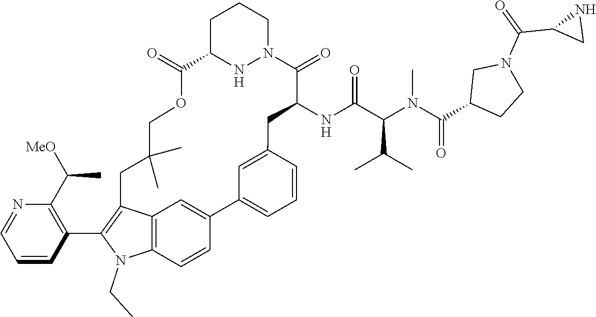 |
| 107 | 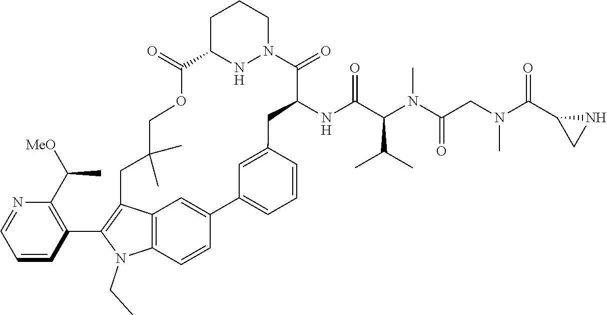 |
| 108* | 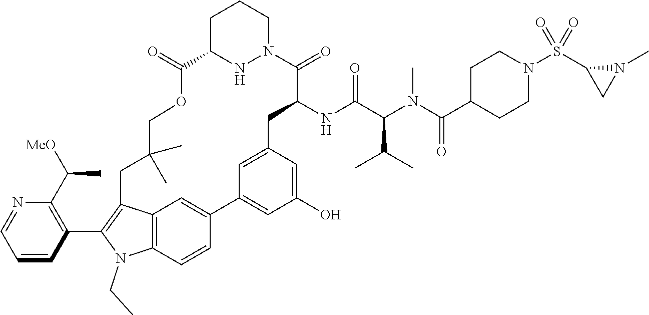 |
| 109 | 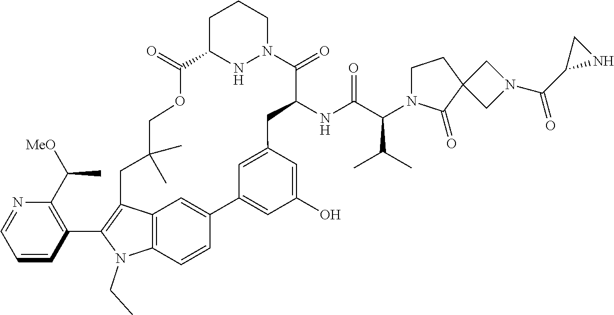 |
| 110 | 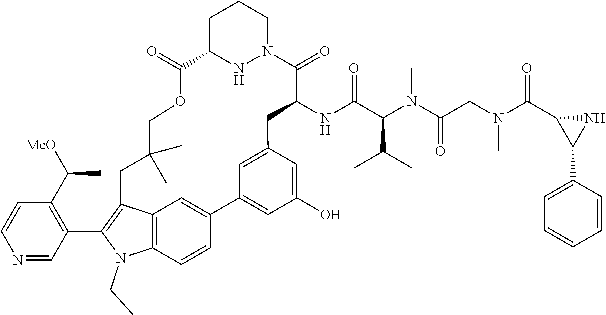 [SMILES: CCn1c(-c2cnccc2[C@H](C)OC)c2c3cc(ccc31)-c1cc(O)cc(c1)C[C@H](NC(=O)[C@H](C(C)C)N(C)C(=O)CN(C)C(=O)[C@@H]1N[C@@H]1c1ccccc1)C(=O)N1CCC[C@H](N1)C(=O)OCC(C)(C)C2] |
| 111 | 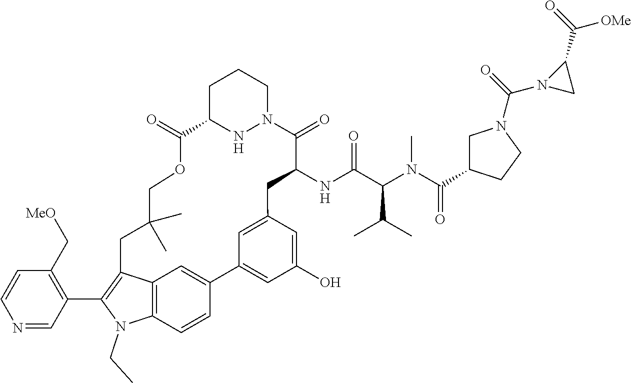 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@H]3C(=O)OC)C1)C(=O)N1CCC[C@H](N1)C(=O)OCC(C)(C)C2] |
| 112 | 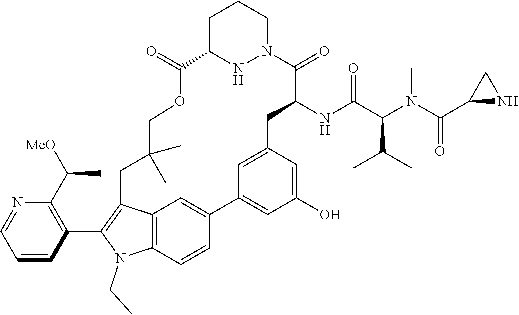 |
| 113 | 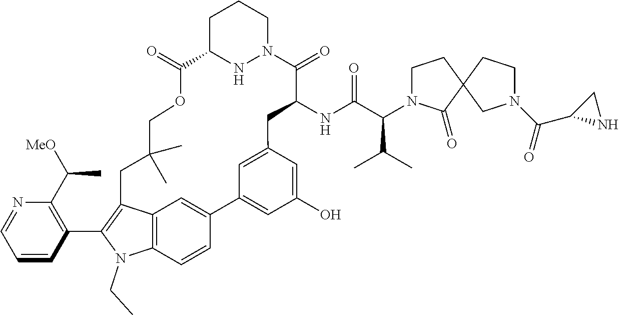 |
| 114 | 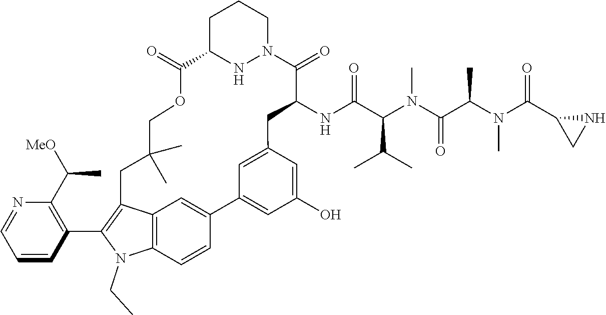 |
| 115 | 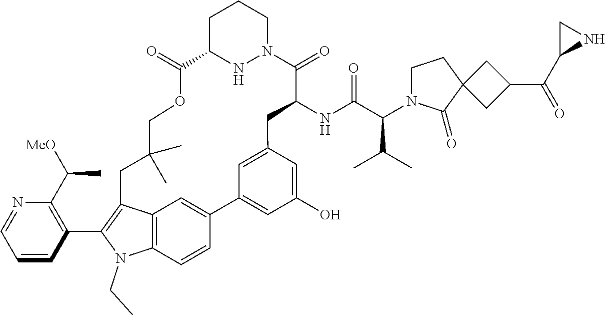 |
| 116 | 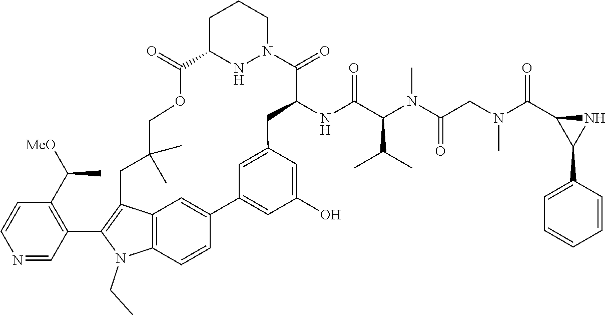 [SMILES: CCn1c(-c2cnccc2[C@H](C)OC)c2c3cc(ccc31)-c1cc(O)cc(c1)C[C@H](NC(=O)[C@H](C(C)C)N(C)C(=O)CN(C)C(=O)[C@H]1N[C@H]1c1ccccc1)C(=O)N1CCC[C@H](N1)C(=O)OCC(C)(C)C2] |
| 117* | 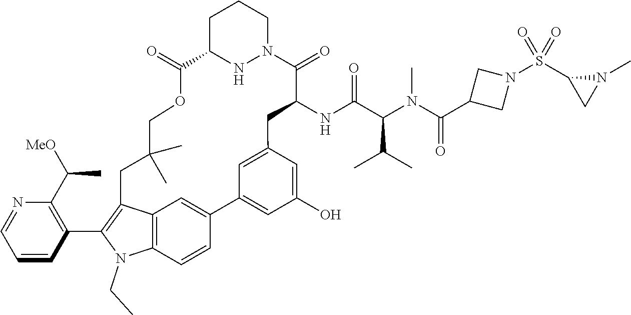 |
| 118* | 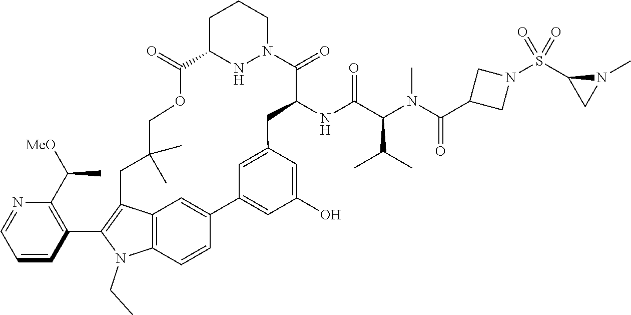 |
| 119* | 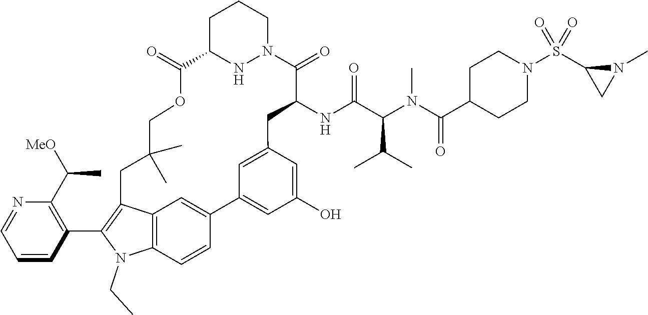 |
| 120 | 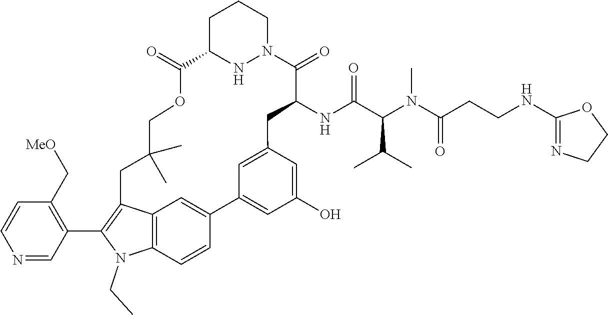 [SMILES: CCn1c(-c2cnccc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| 121 | 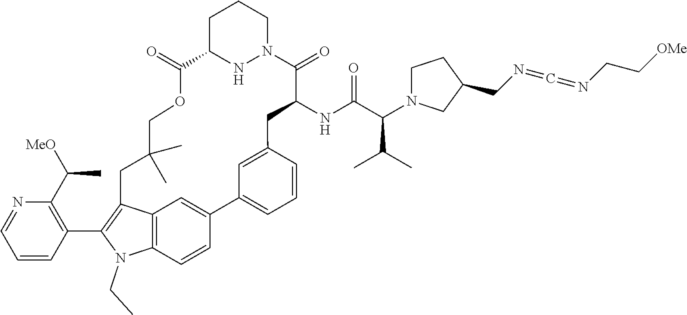 [SMILES: CCn1c(-c2cccnc2[C@H](C)OC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| 122 | 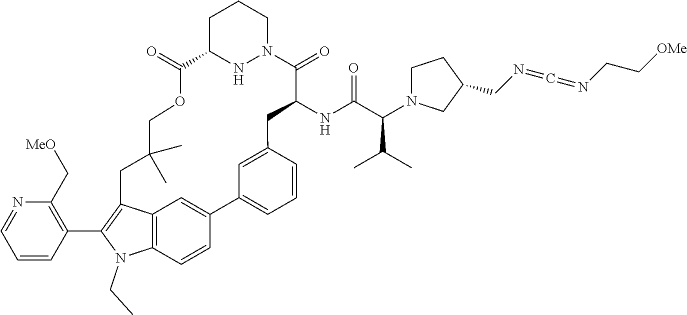 [SMILES: CCn1c(-c2cccnc2COC)c2c3cc(ccc31)-c1cccc(c1)C[C@H](NC(=O)[C@H](C(C)C)N1CC[C@H](CN=C=NCCOC)C1)C(=O)N1CCC[C@H](N1)C(=O)OCC(C)(C)C2] |
| 123 | 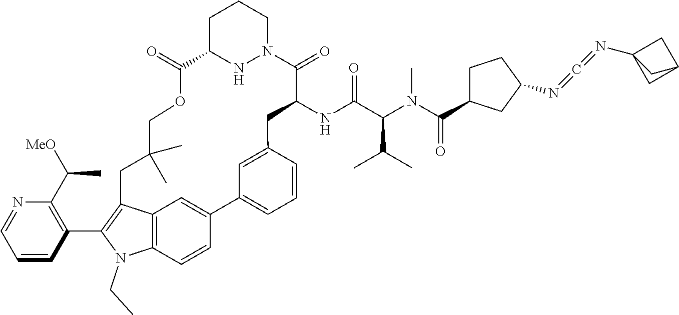 |
| 124 | 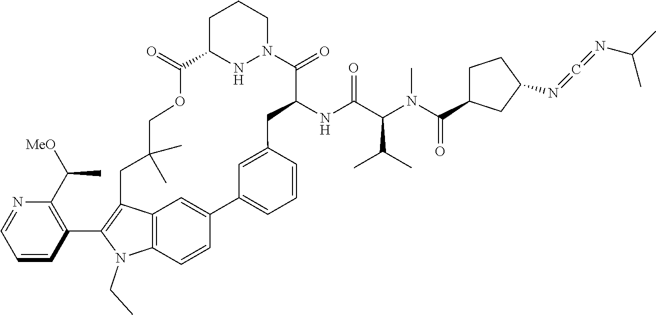 |
| 125 | 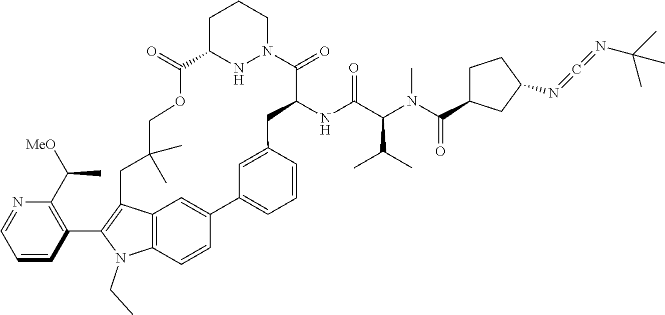 |
| 126 | 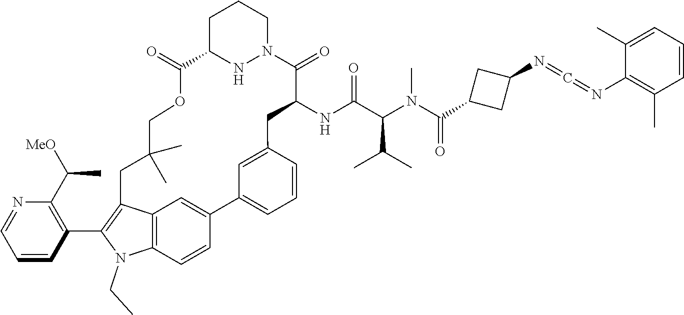 |
| 127 | 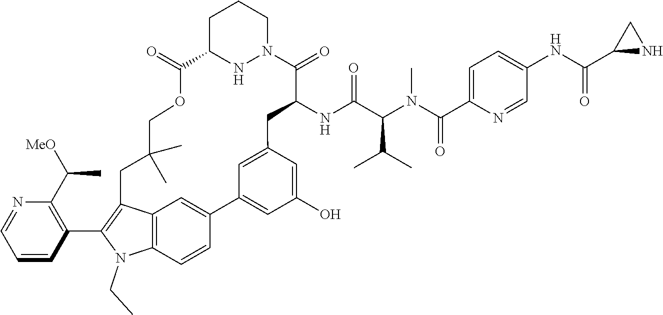 |
| 128 | 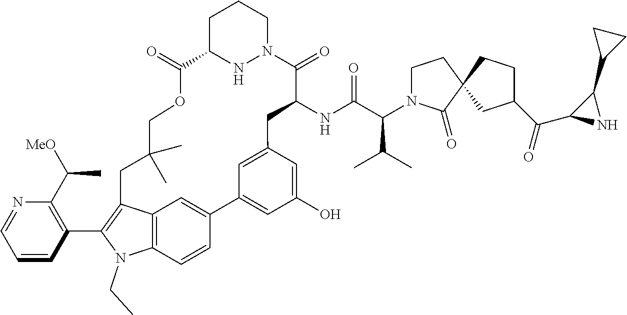 |
| 129 | 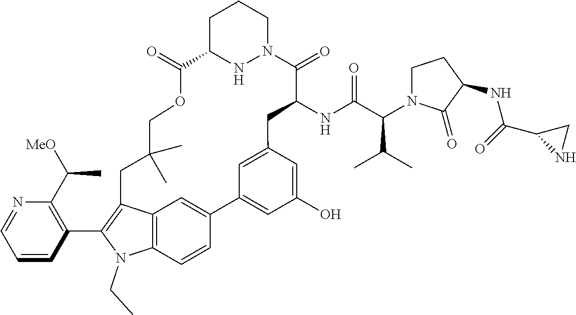 |
| 130 | 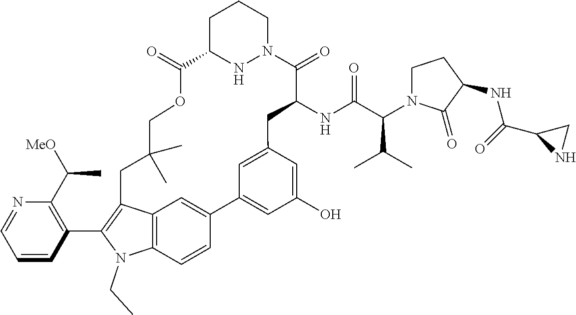 |
| 131 | 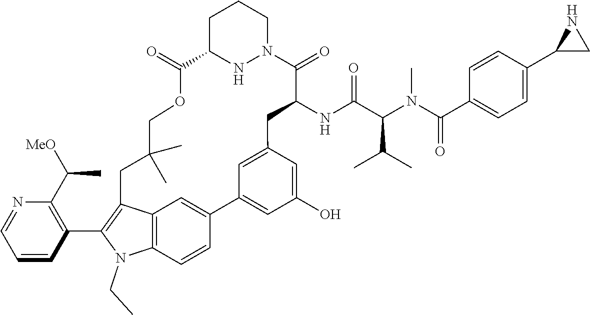 |
| 132 | 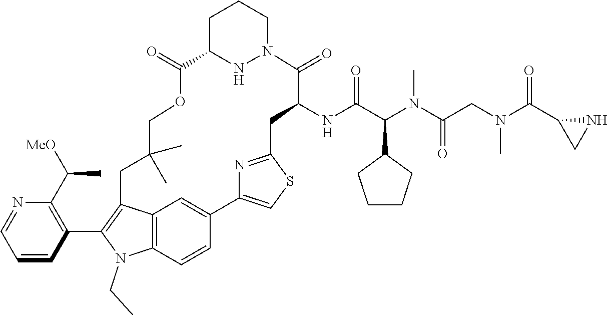 |
| 133 | 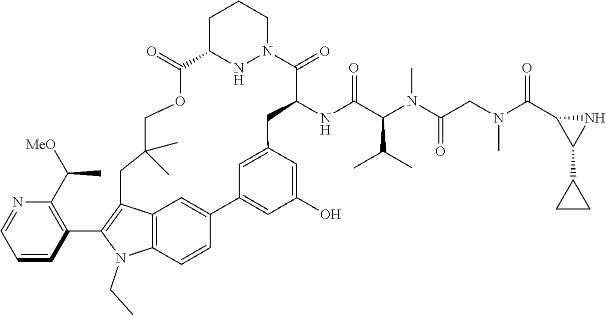 |
| 134 | 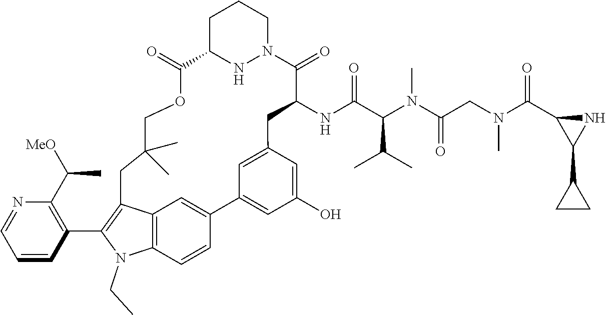 |
| 135 | 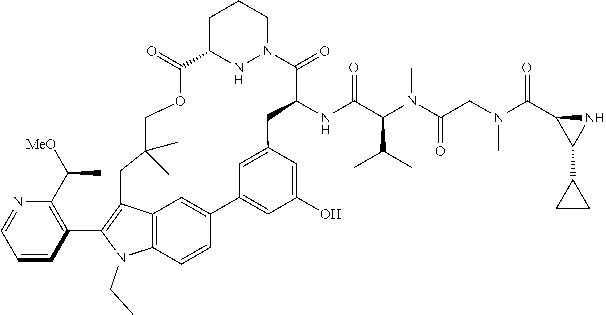 |
| 136 | 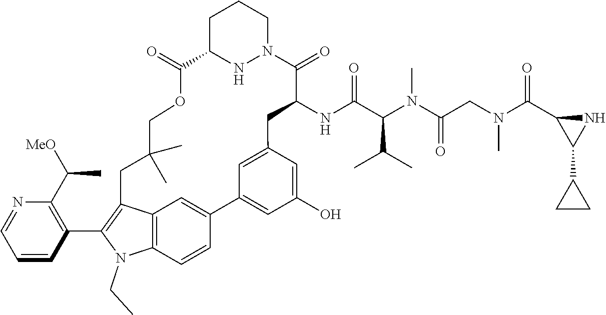 |
| 137 | 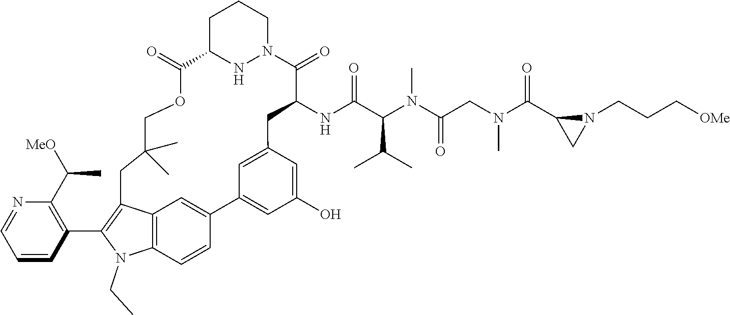 |
| 138 | 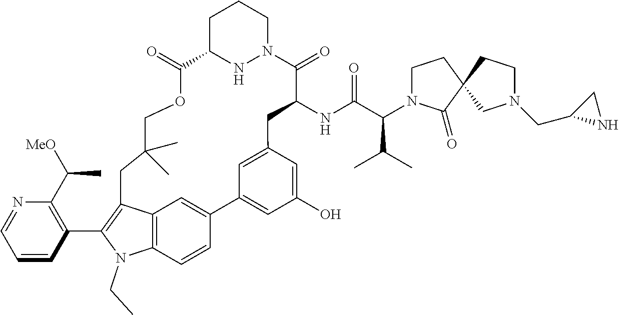 |
| 139 | 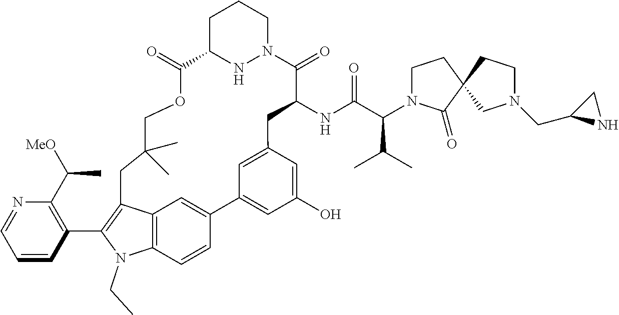 |
| 140 | 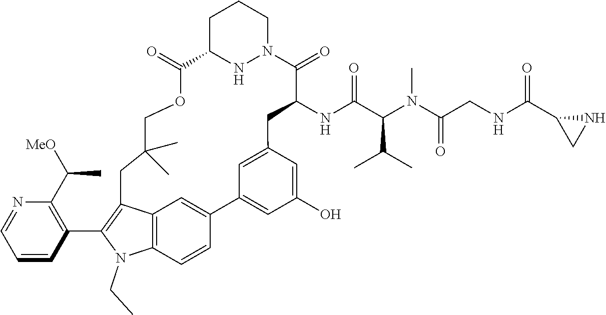 |
| 141 | 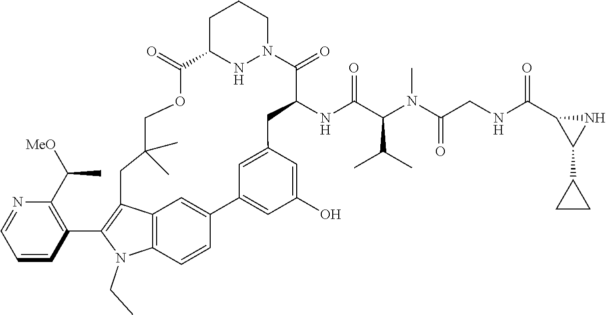 |
| 142 | 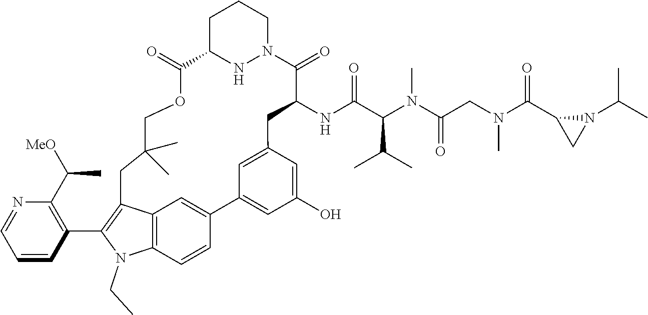 |
| 143 | 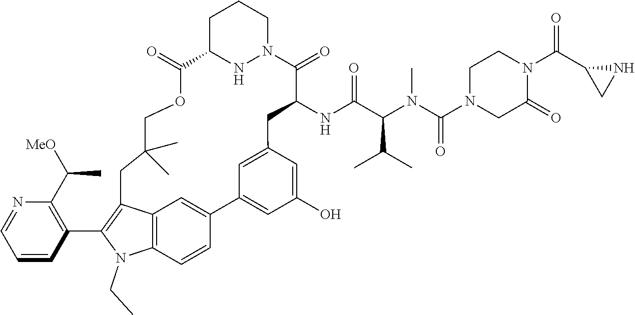 |
| 144 | 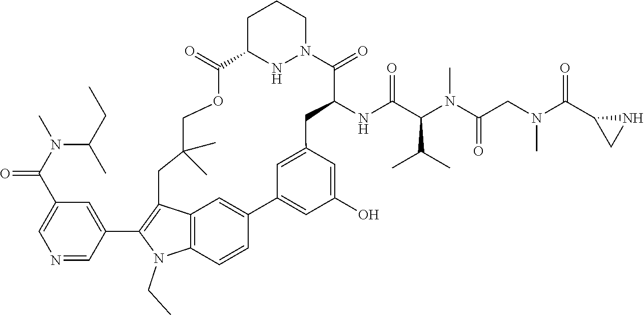 [SMILES: CCC(C)N(C)C(=O)c1cncc(-c2c3c4cc(ccc4n2CC)-c2cc(O)cc(c2)C[C@H](NC(=O)[C@H](C(C)C)N(C)C(=O)CN(C)C(=O)[C@H]2CN2)C(=O)N2CCC[C@H](N2)C(=O)OCC(C)(C)C3)c1] |
| 145 | 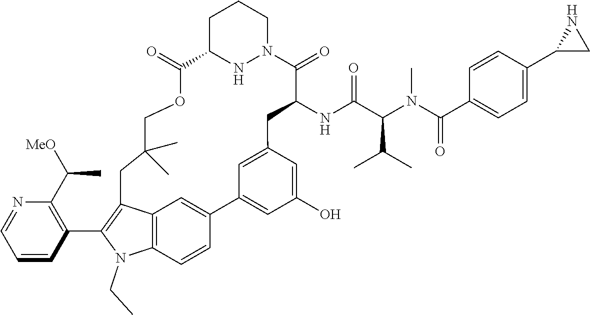 |
| 146 | 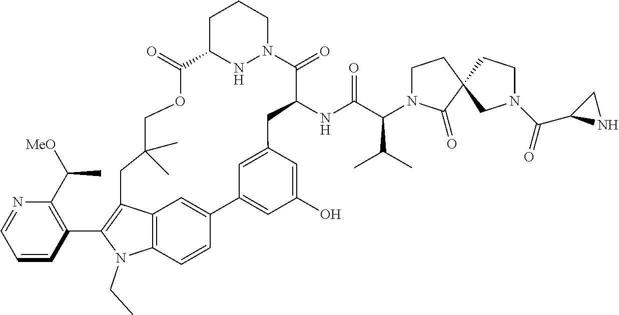 |
| 147 | 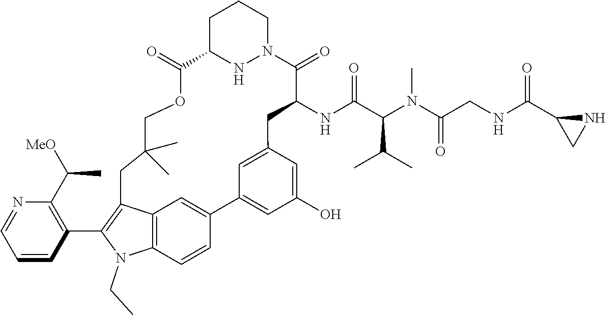 |
| 148 | 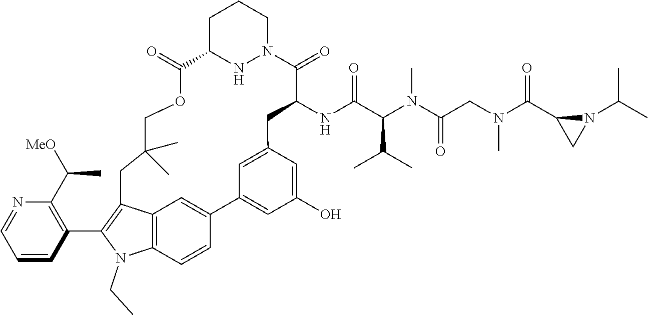 |
| 149 | 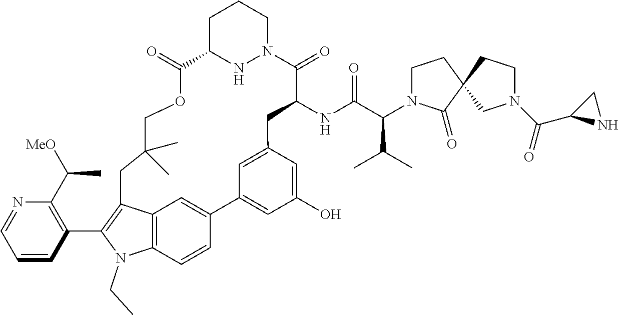 |
| 150 | 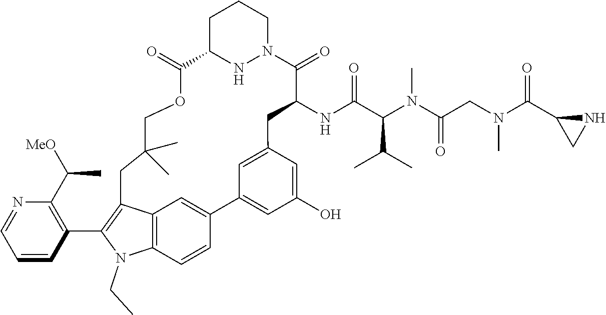 |
| 151 | 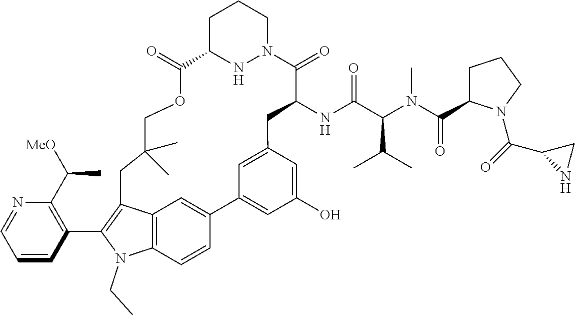 |
| 152 | 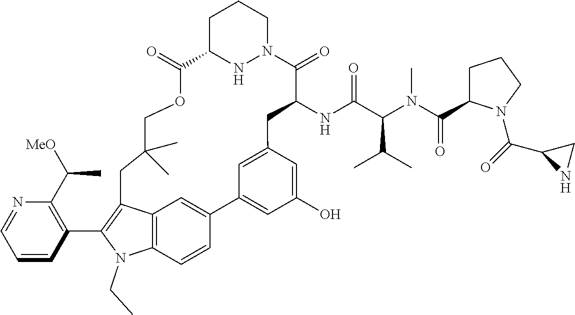 |
| 153 | 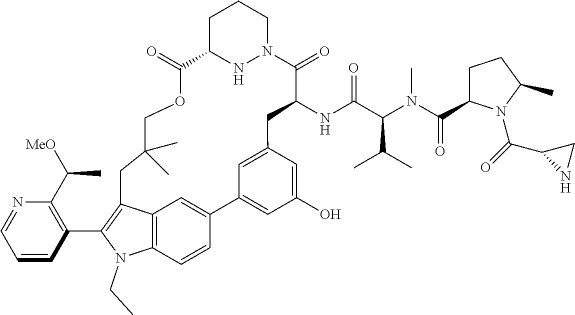 |
| 154 | 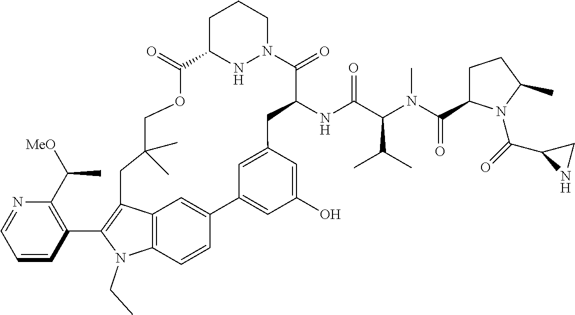 |
| 155 | 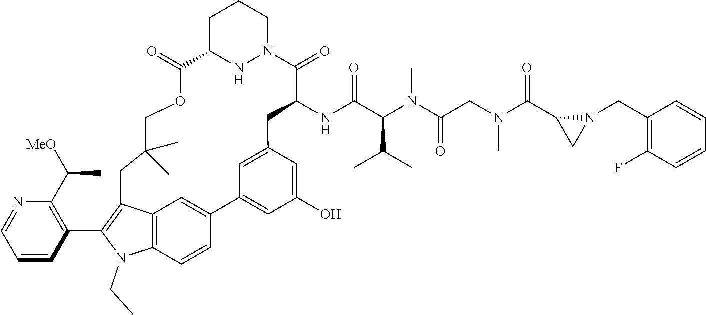 |
| 156 | 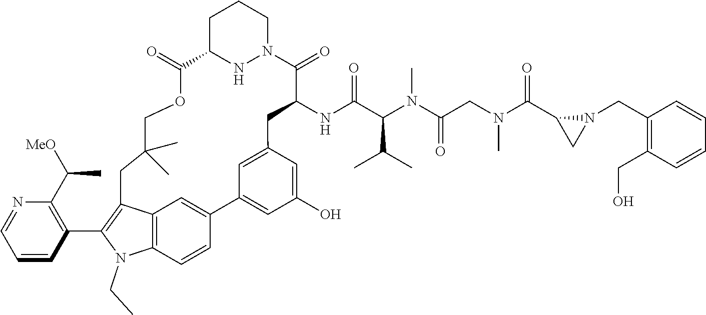 |
| 157 | 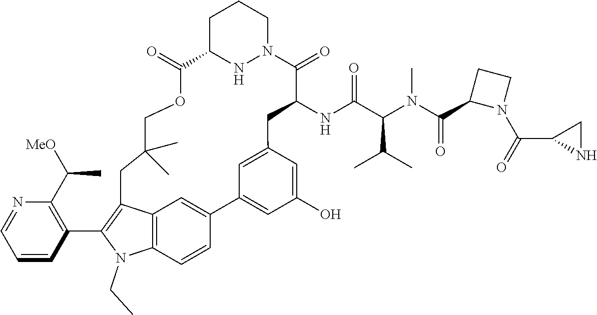 |
| 158 | 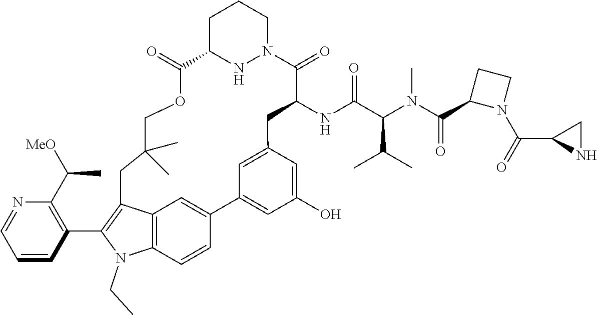 |
| 159 | 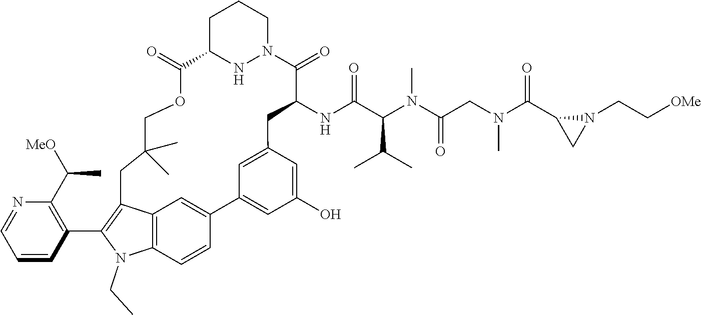 |
| 160 | 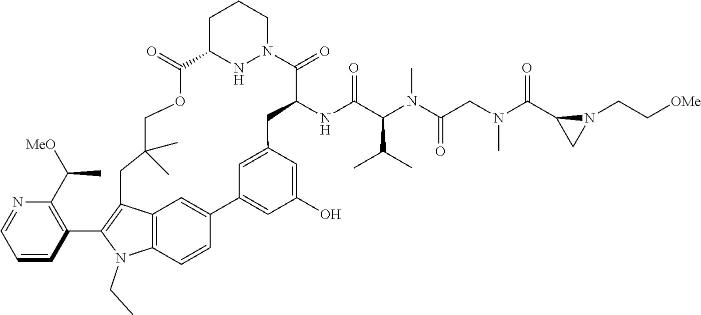 |
| 161 | 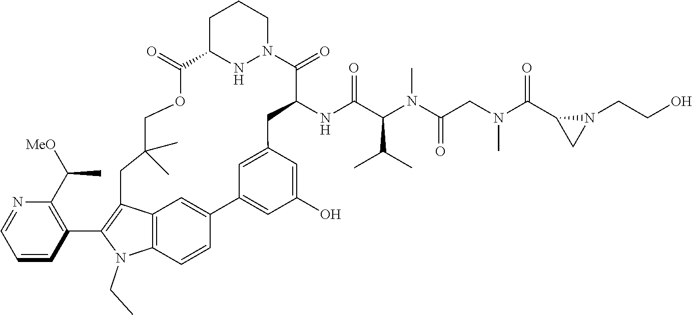 |
| 162 | 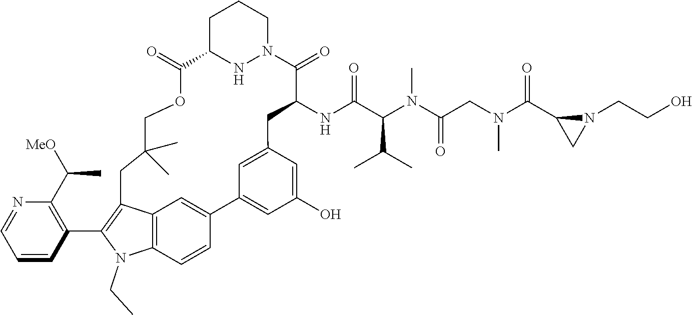 |
| 163 | 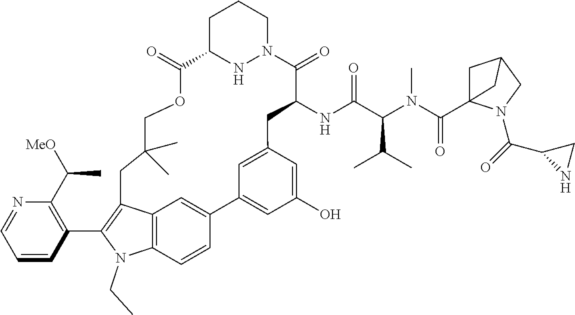 |
| 164 | 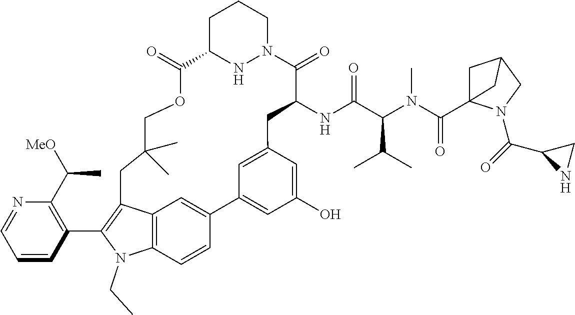 |
| 165 | 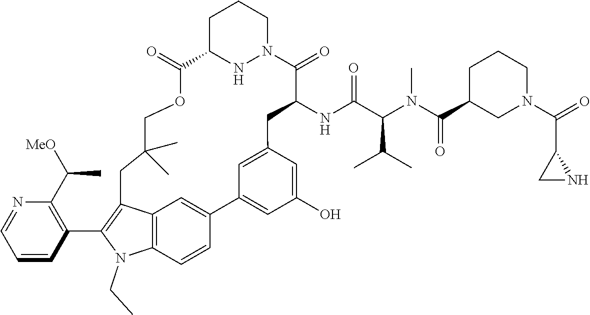 |
| 166 | 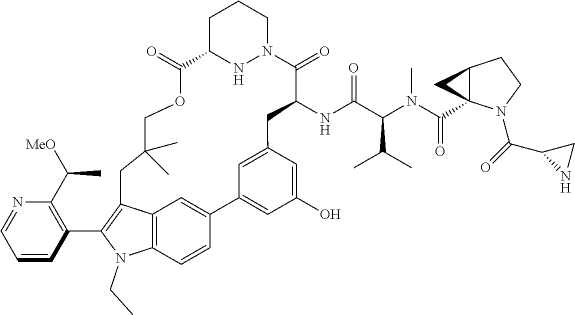 |
| 167 | 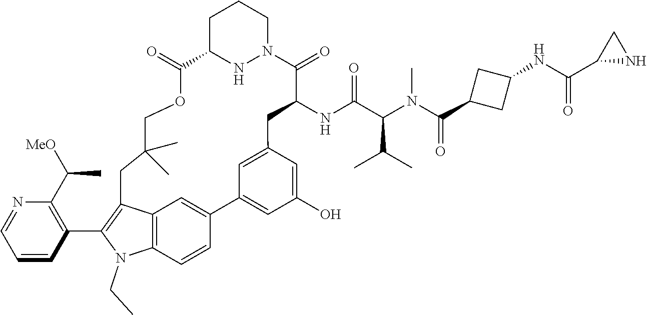 |
| 168 | 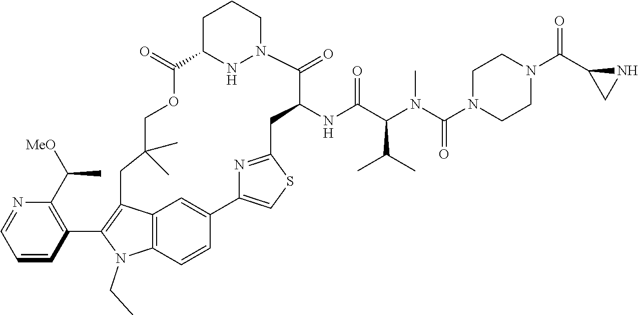 |
| 169 | 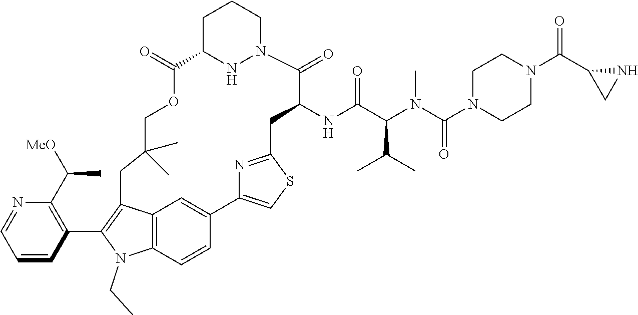 |
| 170 | 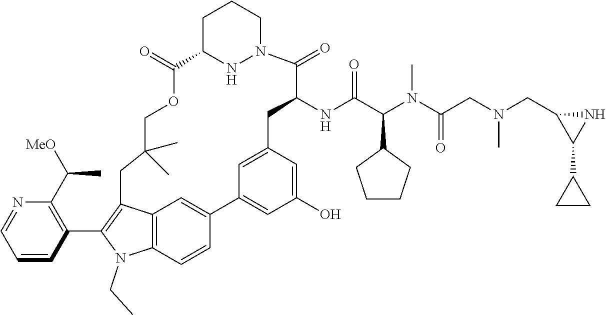 |
| 171 | 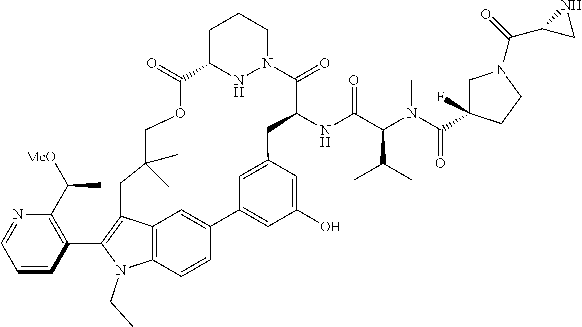 |
| 172 | 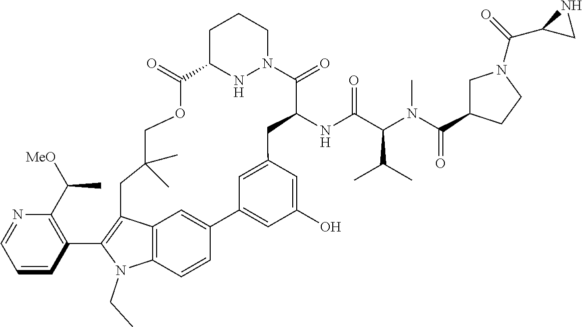 |
| 173 | 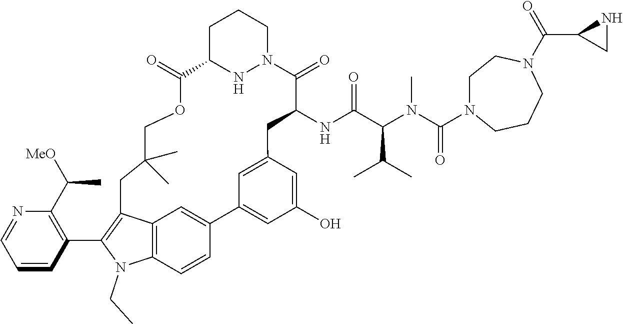 |
| 174 | 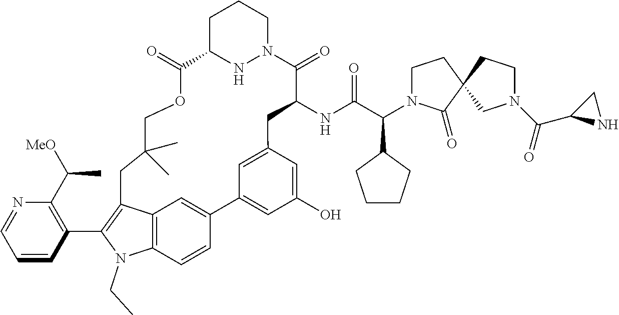 |
| 175 | 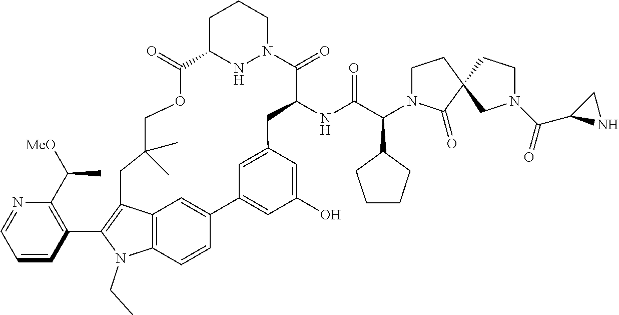 |
| 176 | 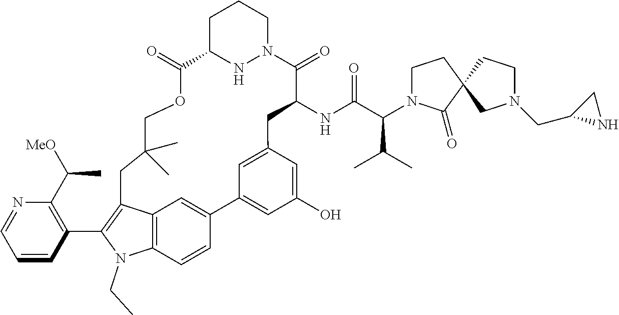 |
| 177 | 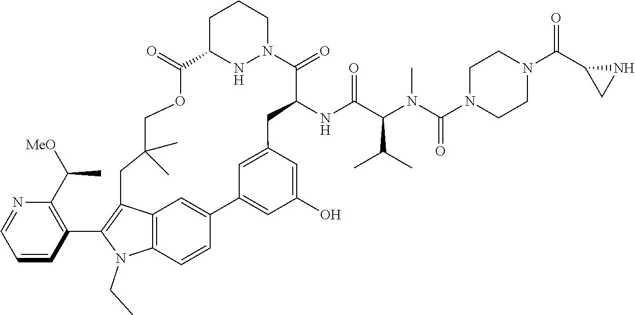 |
| 178 | 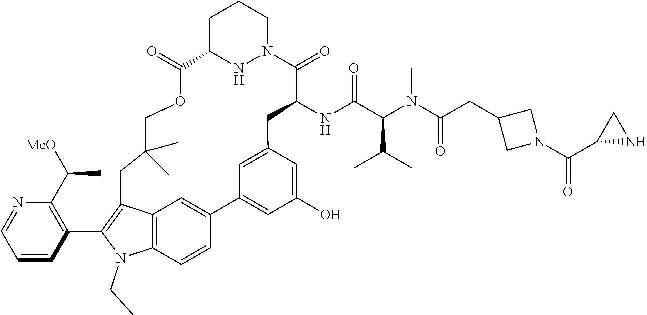 |
| 179 | 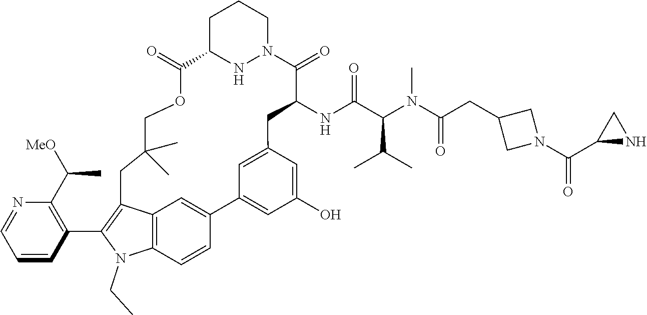 |
| 180 | 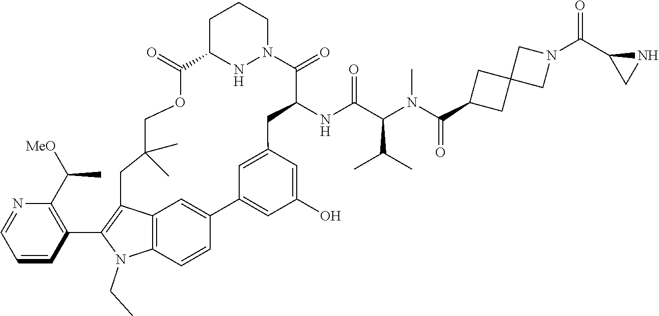 |
| 181 | 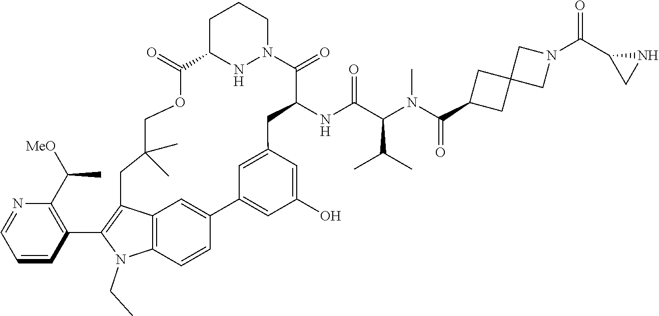 |
| 182 | 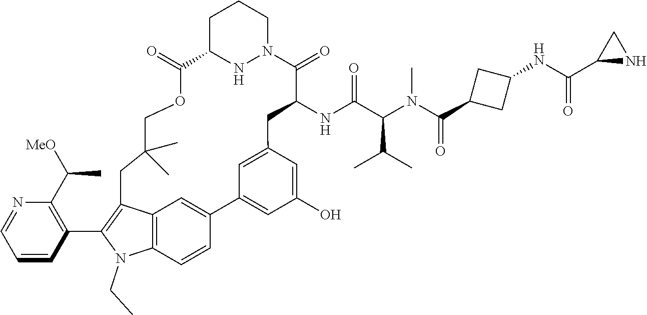 |
| 183 | 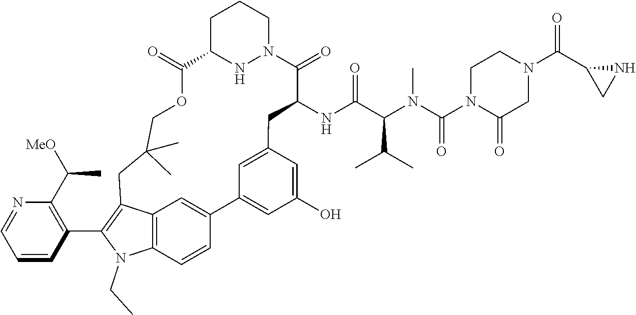 |
| 184 | 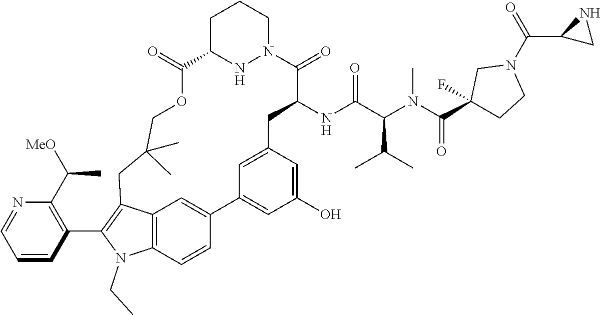 |
| 185 | 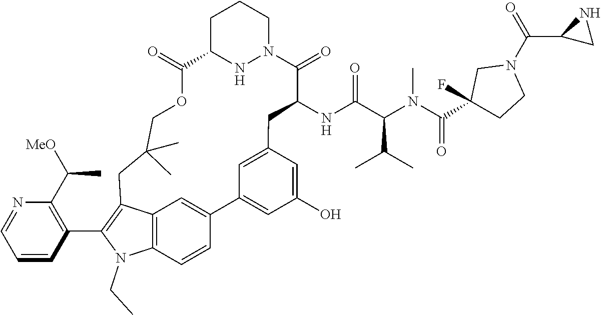 |
| 186 | 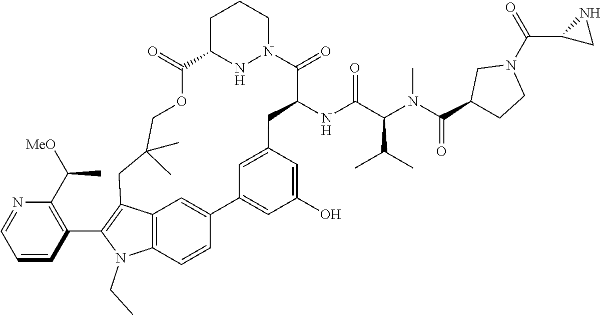 |
| 187 | 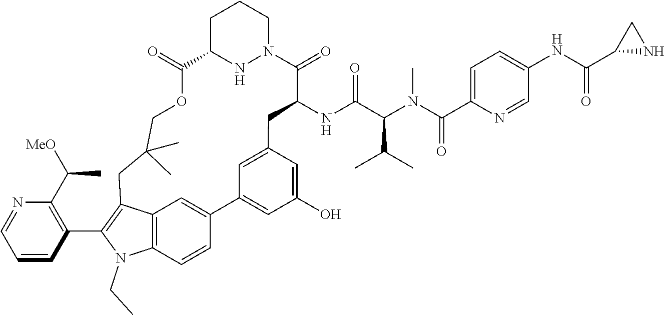 |
| 188 | 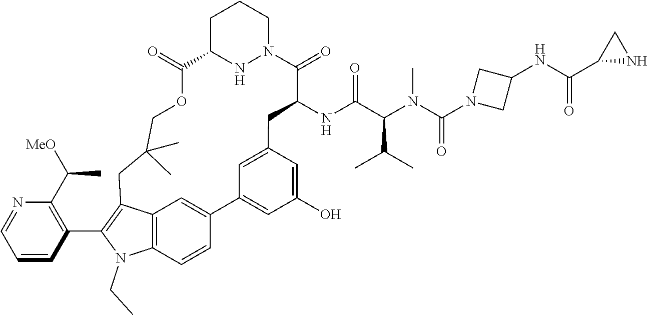 |
| 189 | 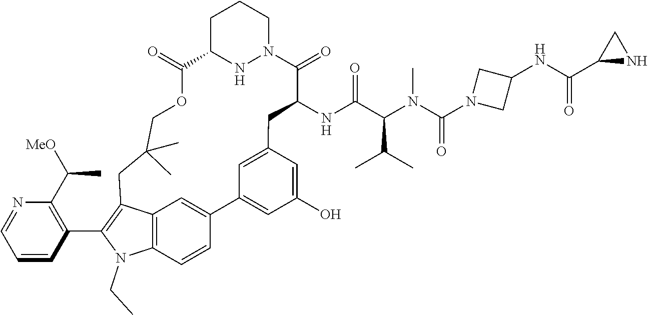 |
| 190 | 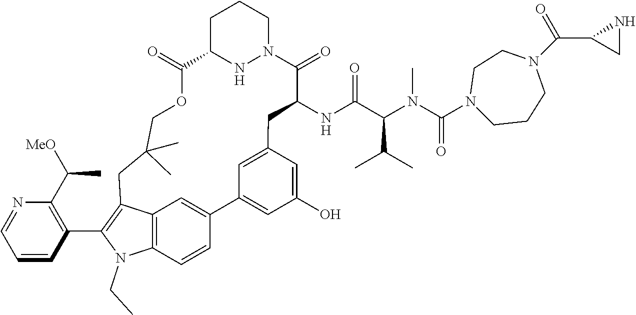 |
| 191 | 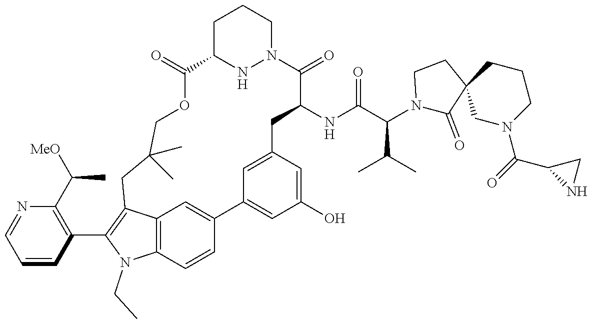 |
| 192 | 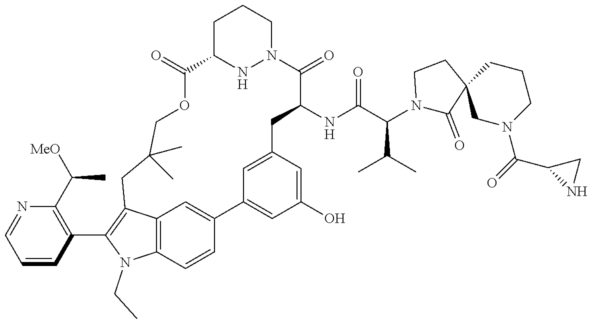 |
| 193 | 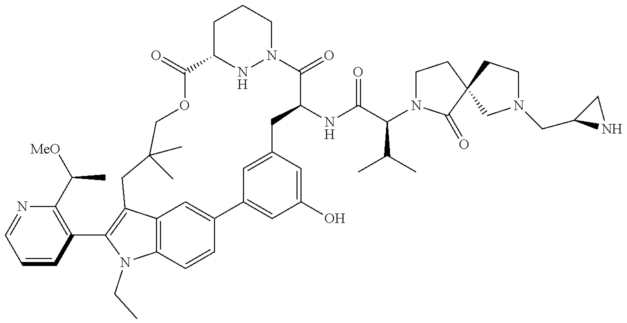 |
| 194 | 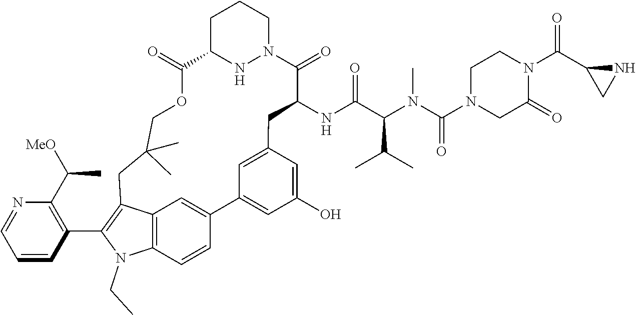 |
| 195 | 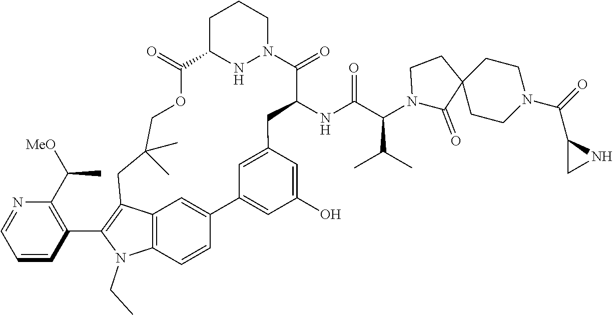 |
| 196 | 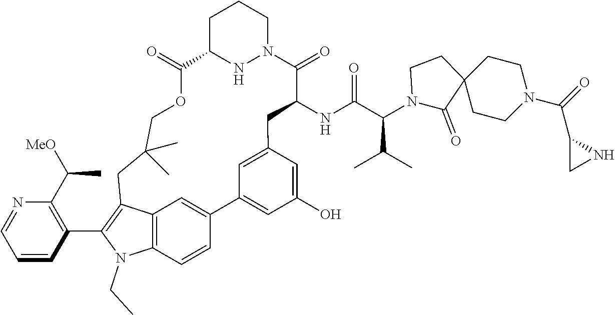 |
| 197 | 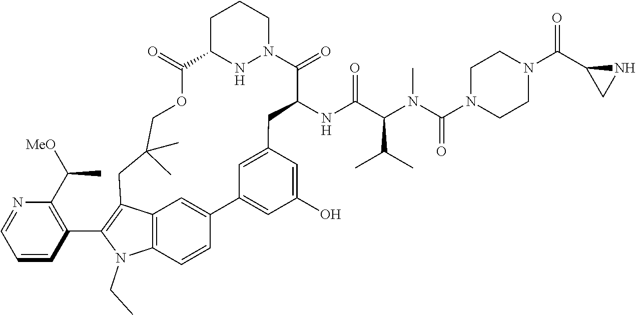 |
| 198 | 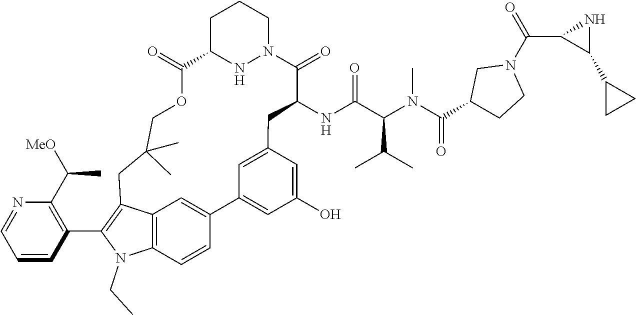 |
| 199 | 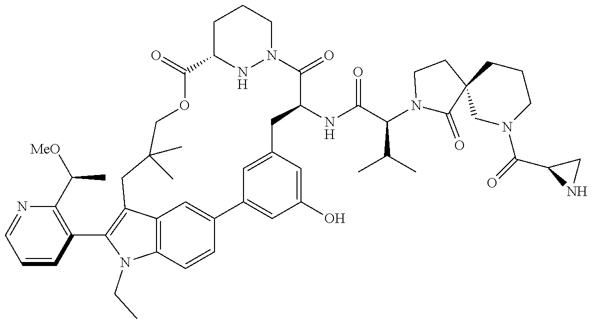 |
| 200 | 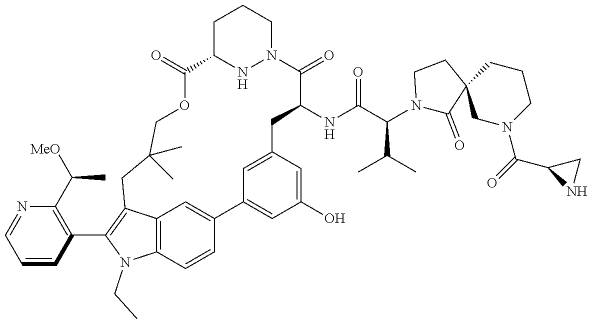 |
| 201 | 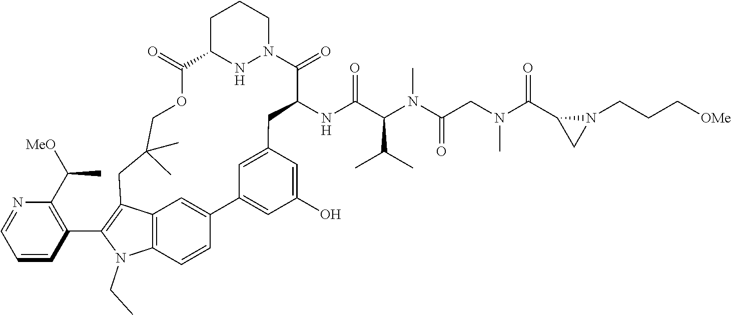 |
| 202 | 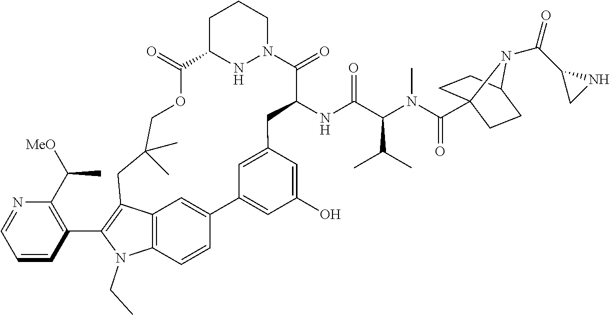 |
| 203 | 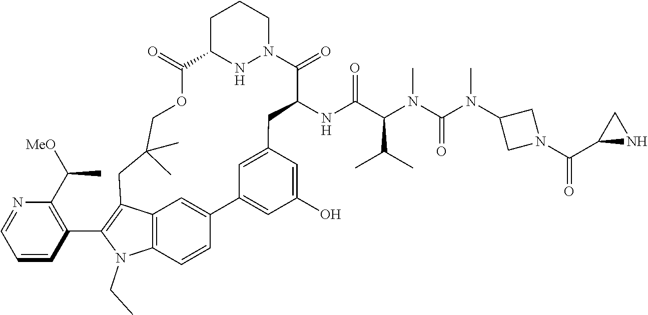 |
| 204 | 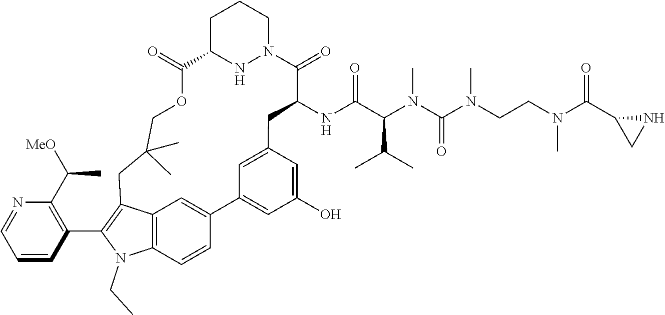 |
| 205 | 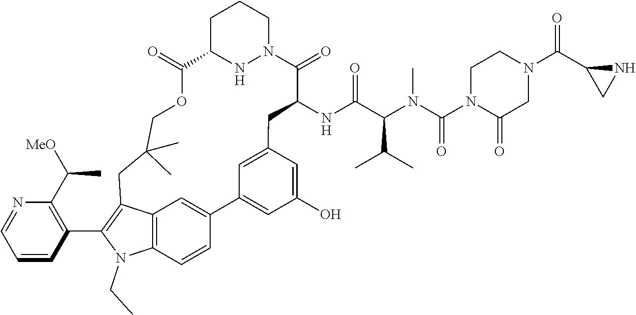 |
| 206 | 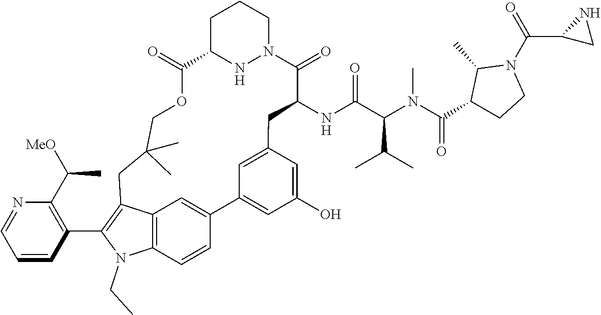 |
| 207 | 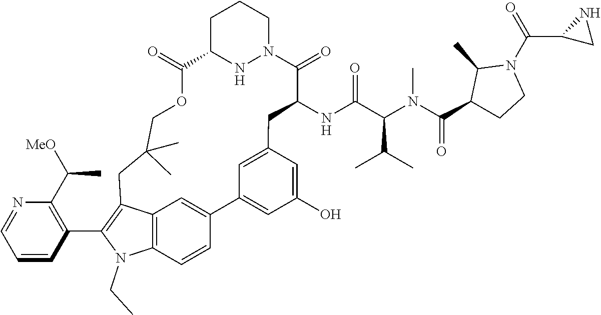 |
| 208 | 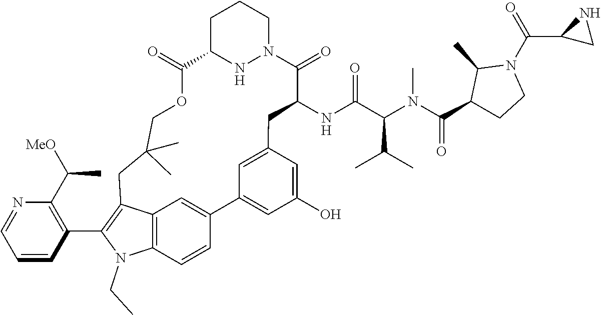 |
| 209 | 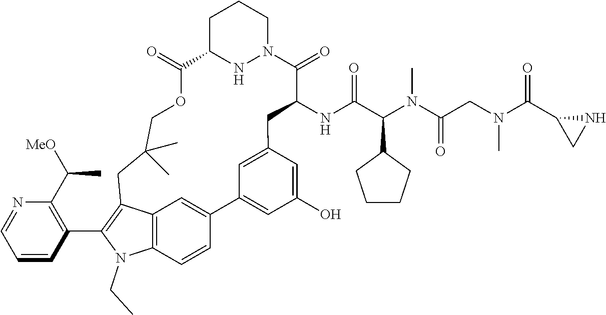 |
| 210 | 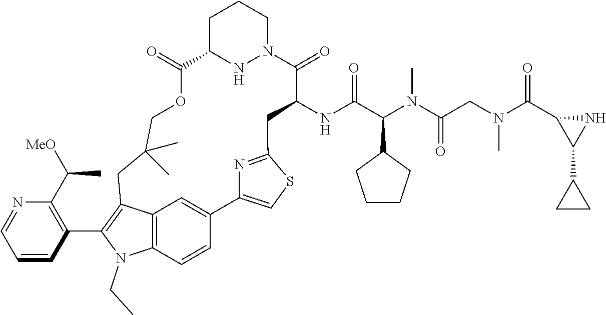 |
| 211 | 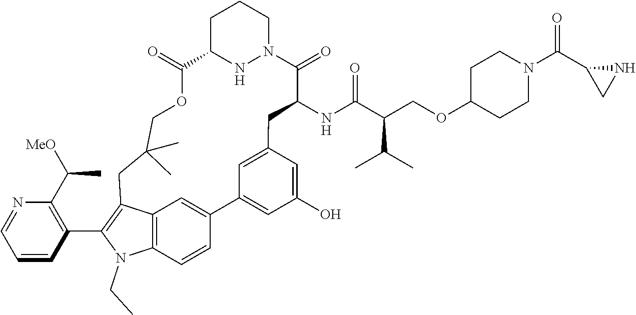 |
| 212 | 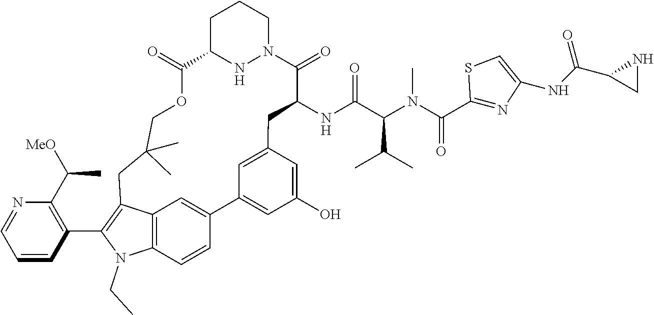 |
| 213 | 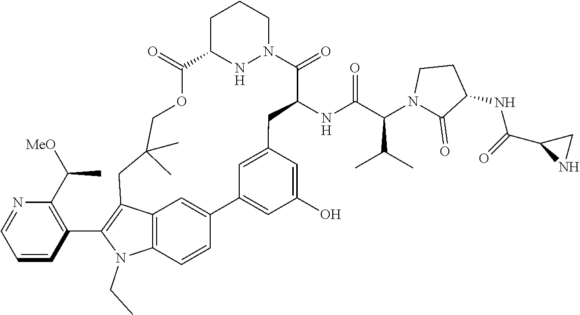 |
| 214 | 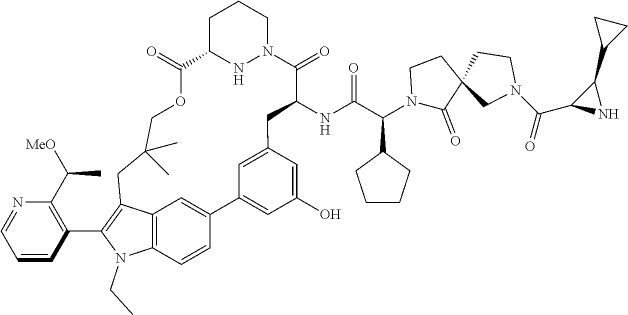 |
| 215 | 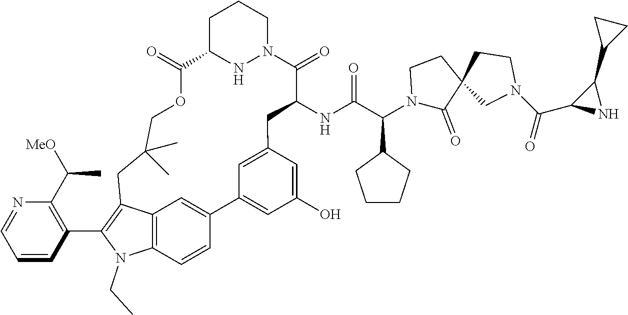 |
| 216 | 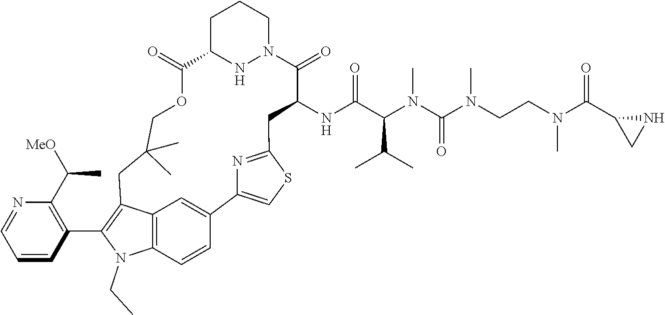 |
| 217 | 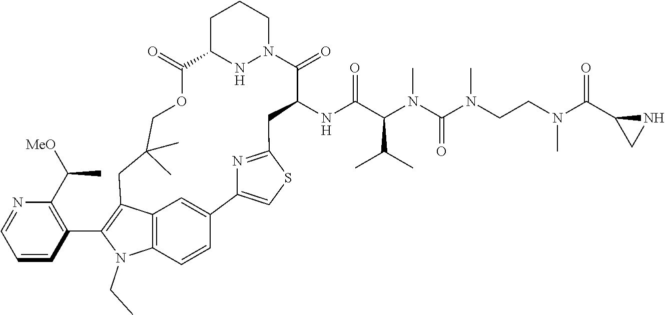 |
| 218 | 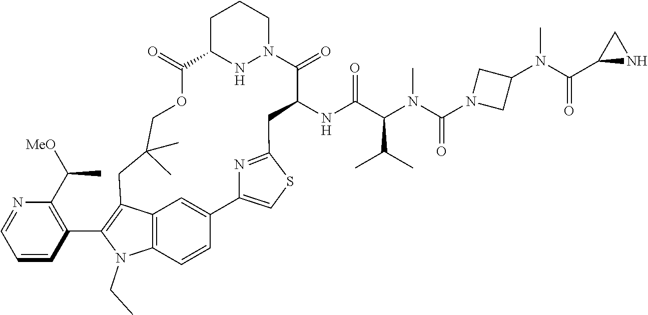 |
| 219 | 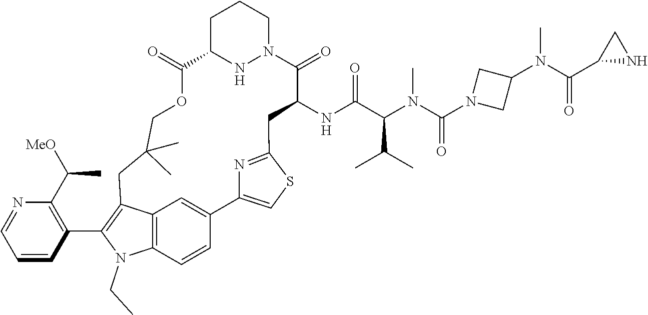 |
| 220 | 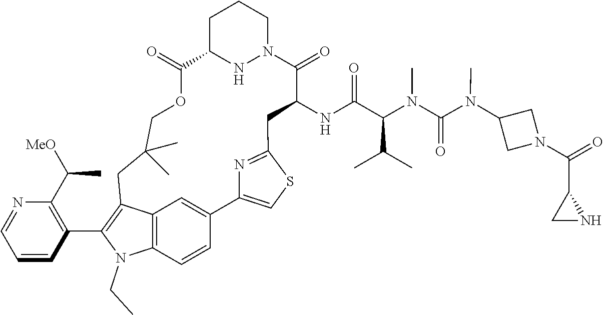 |
| 221 | 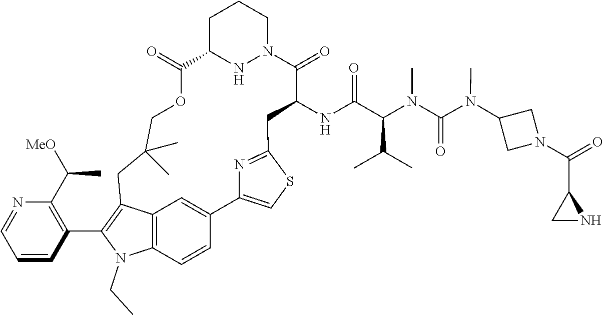 |
| 222 | 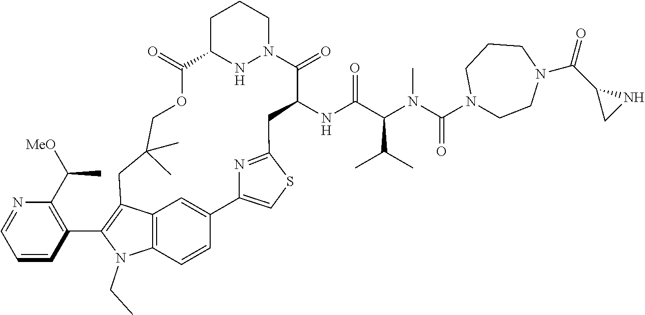 |
| 223 | 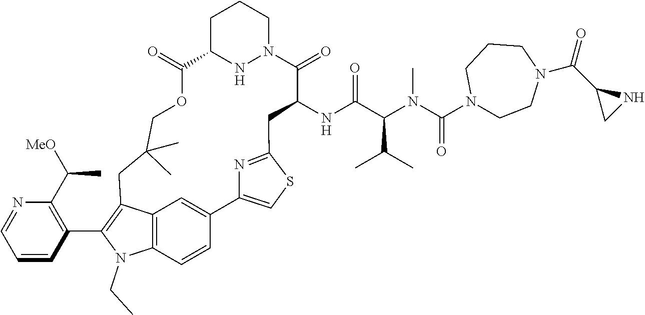 |
| 224 | 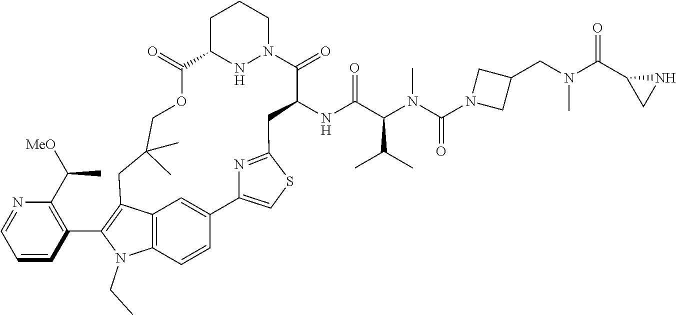 |
| 225 | 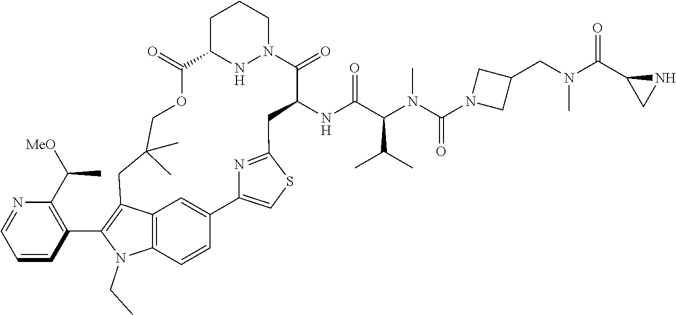 |
| 226 | 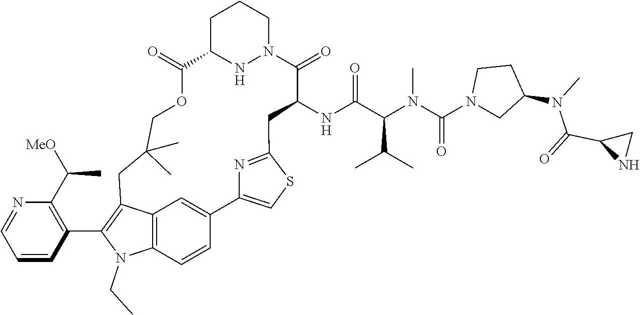 |
| 227 | 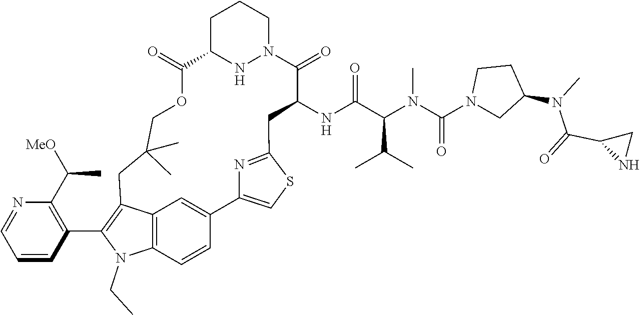 |
| 228 | 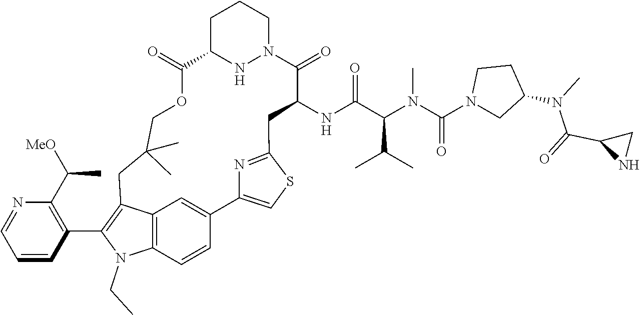 |
| 229 | 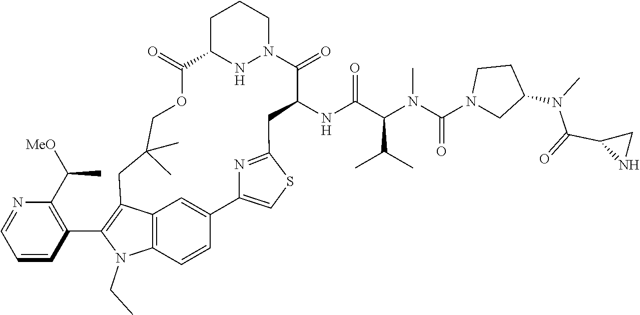 |
| 230 | 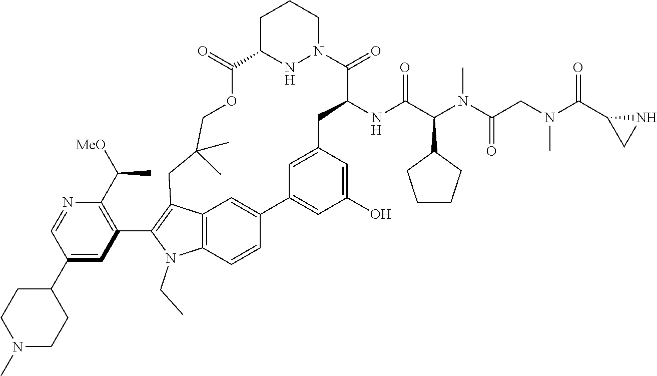 |
| 231 | 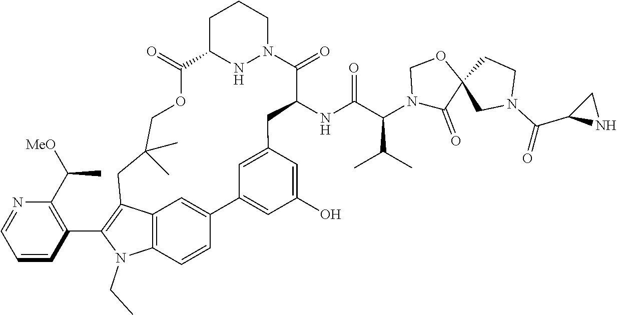 |
| 232 | 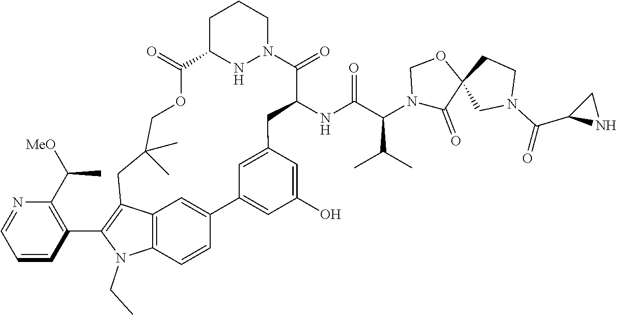 |
| 233 | 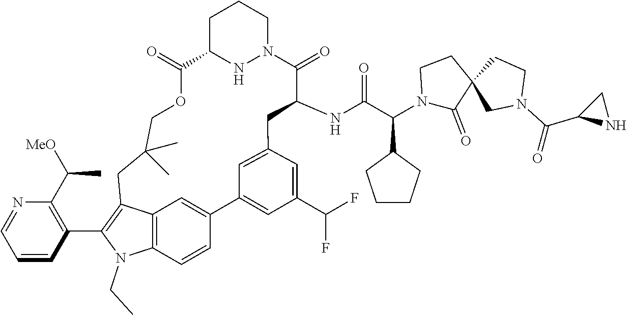 |
| 234 | 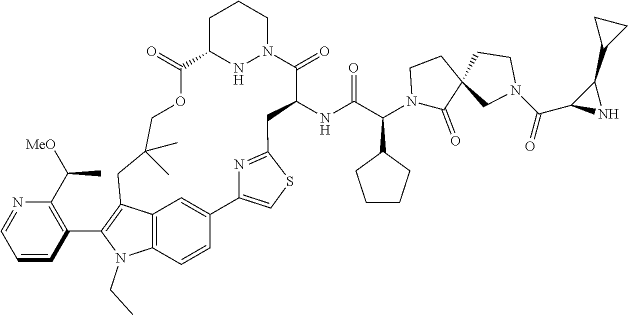 |
| 235 | 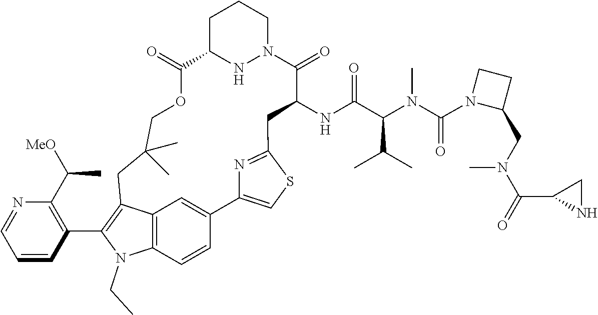 |
| 236 | 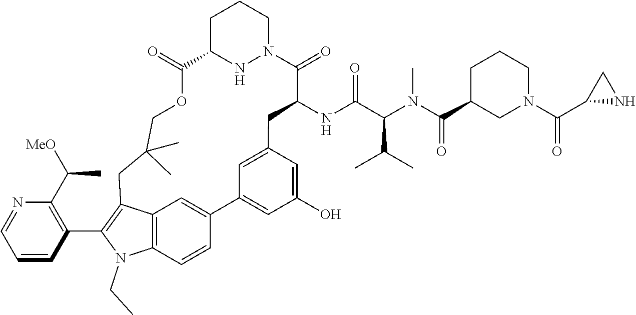 |
| 237 | 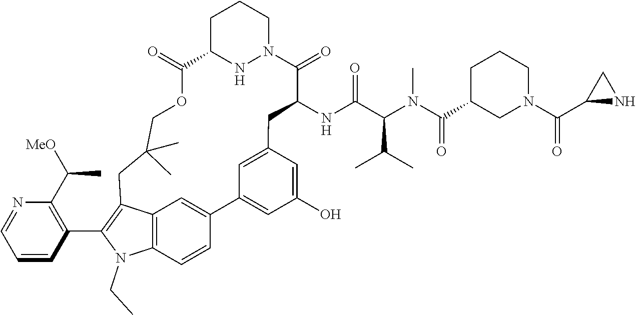 |
| 238 | 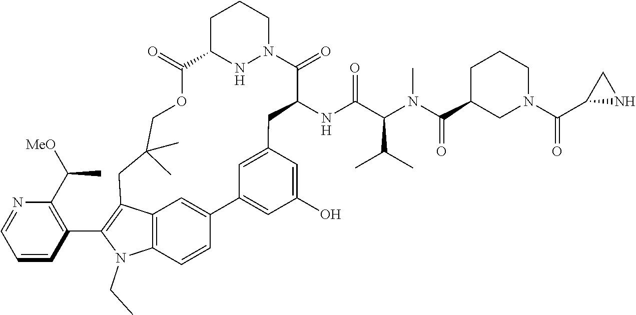 |
| 239 | 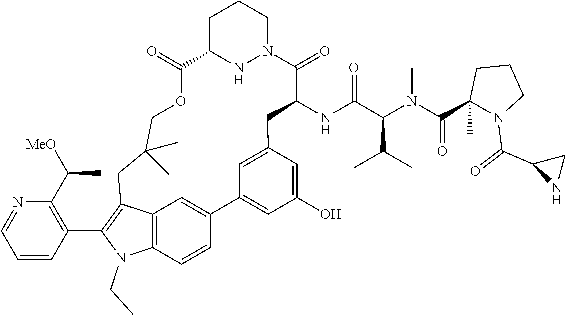 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCCN1C(=O)[C@H]1CN1)C(=O)N1CCC[C@H](N1)C(=O)OCC(C)(C)C2] |
| 240 | 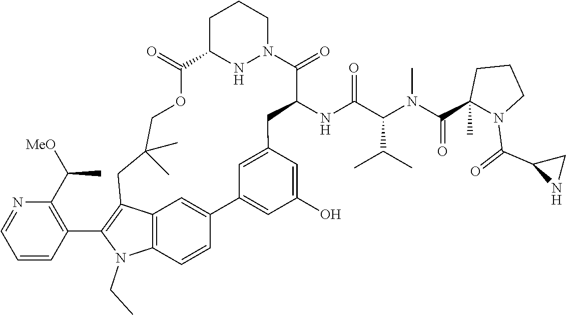 [SMILES: CCn1c(-c2cccnc2[C@H](C)OC)c2c3cc(ccc31)-c1cc(O)cc(c1)C[C@H](NC(=O)[C@@H](C(C)C)N(C)C(=O)[C@@]1(C)CCCN1C(=O)[C@H]1CN1)C(=O)N1CCC[C@H](N1)C(=O)OCC(C)(C)C2] |
| 241 | 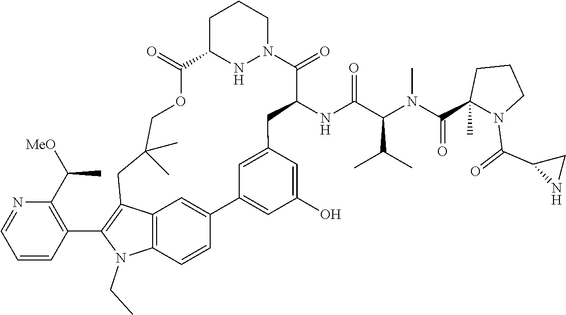 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCCN1C(=O)[C@@H]1CN1)C(=O)N1CCC[C@H](N1)C(=O)OCC(C)(C)C2] |
| 242 | 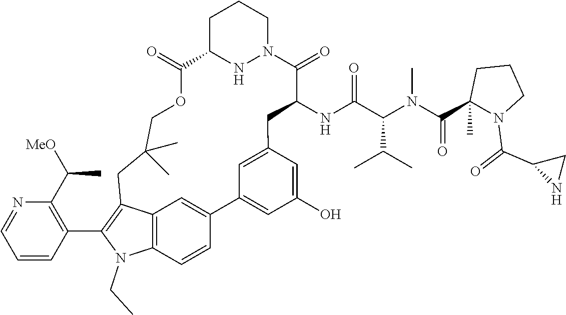 [SMILES: CCn1c(-c2cccnc2[C@H](C)OC)c2c3cc(ccc31)-c1cc(O)cc(c1)C[C@H](NC(=O)[C@@H](C(C)C)N(C)C(=O)[C@@]1(C)CCCN1C(=O)[C@@H]1CN1)C(=O)N1CCC[C@H](N1)C(=O)OCC(C)(C)C2] |
| 243 | 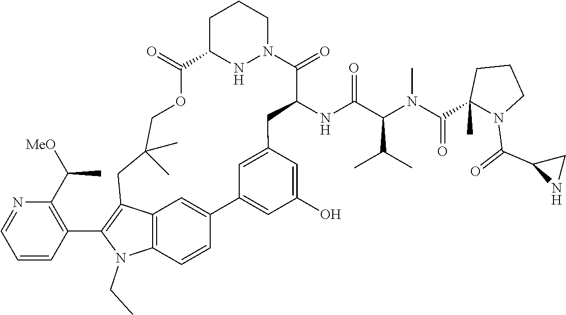 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]1(C)CCCN1C(=O)[C@H]1CN1)C(=O)N1CCC[C@H](N1)C(=O)OCC(C)(C)C2] |
| 244 | 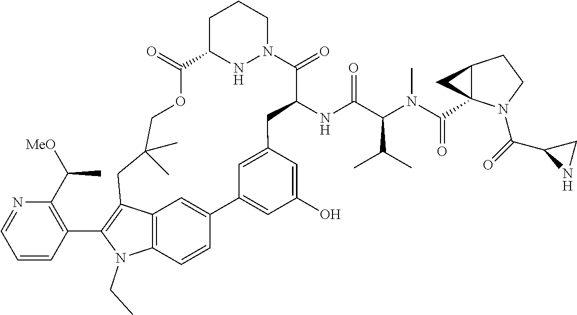 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13C[C@H]1CCN3C(=O)[C@H]1CN1)C(=O)N1CCC[C@H](N1)C(=O)OCC(C)(C)C2] |
| 245 | 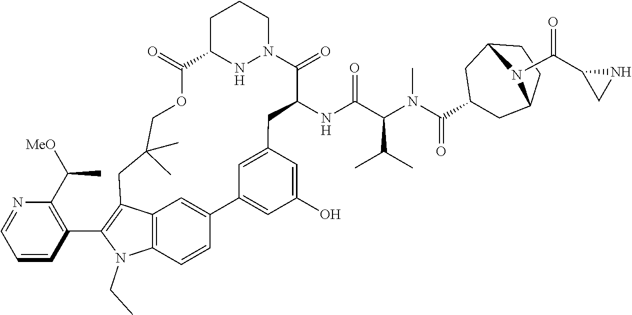 |
| 246 | 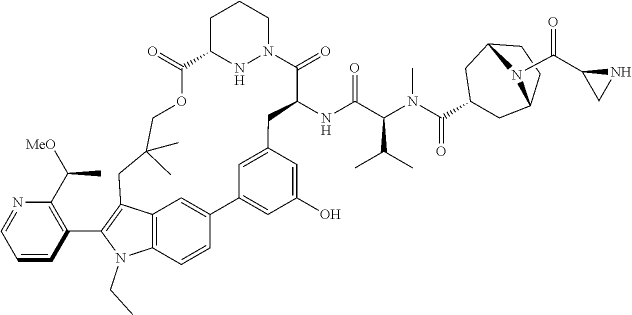 |
| 247 | 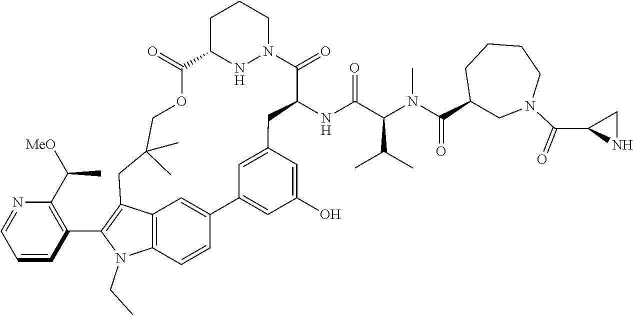 |
| 248 | 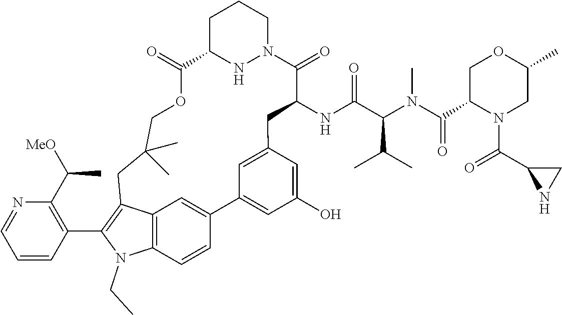 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CO[C@H](C)CN1C(=O)[C@H]1CN1)C(=O)N1CCC[C@H](N1)C(=O)OCC(C)(C)C2] |
| 249 | 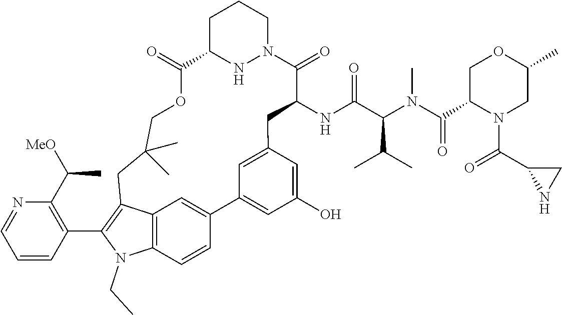 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CO[C@H](C)CN1C(=O)[C@@H]1CN1)C(=O)N1CCC[C@H](N1)C(=O)OCC(C)(C)C2] |
| 250 | 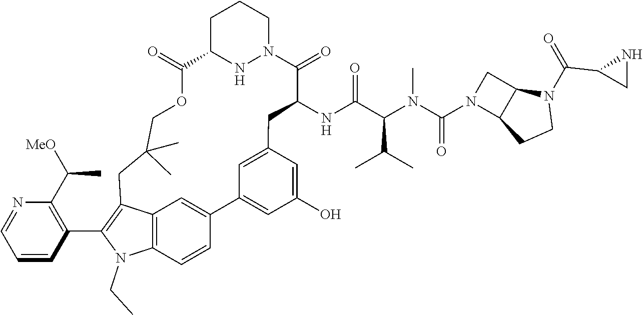 |
| 251 | 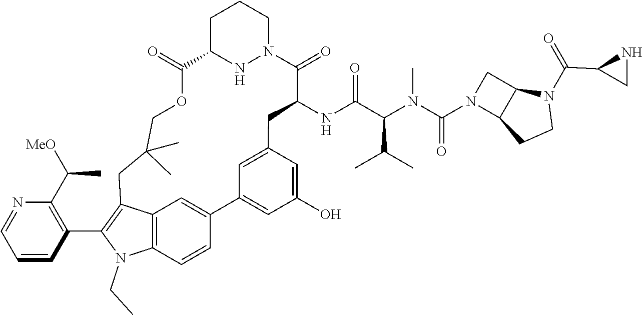 |
| 252 | 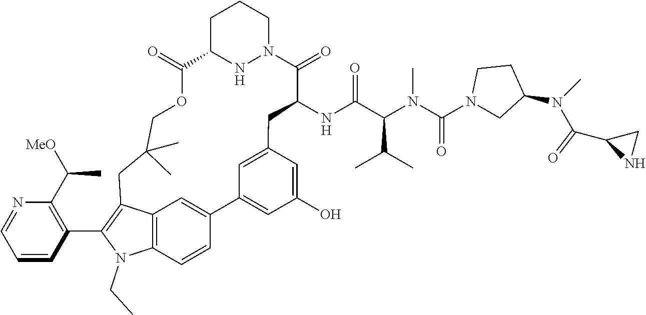 |
| 253 | 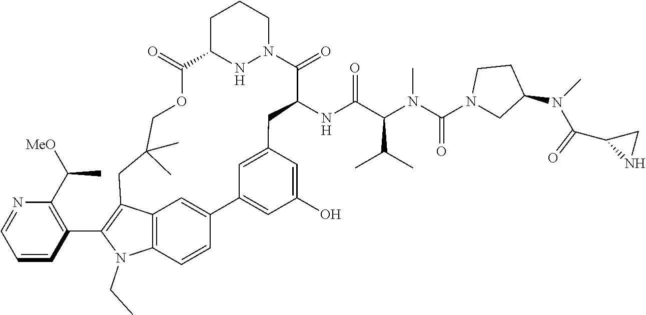 |
| 254 | 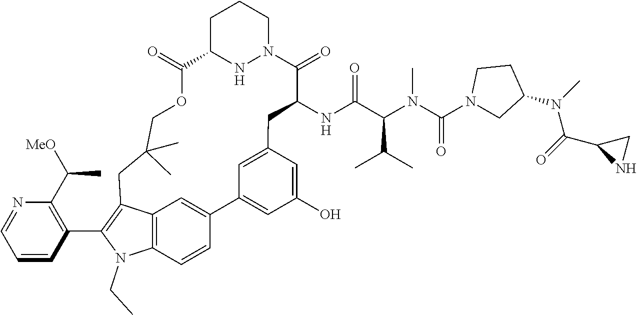 |
| 255 | 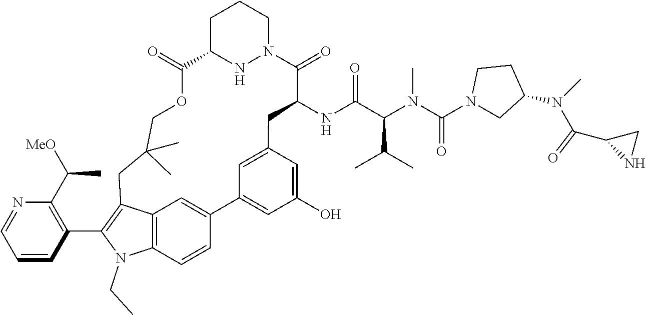 |
| 256 | 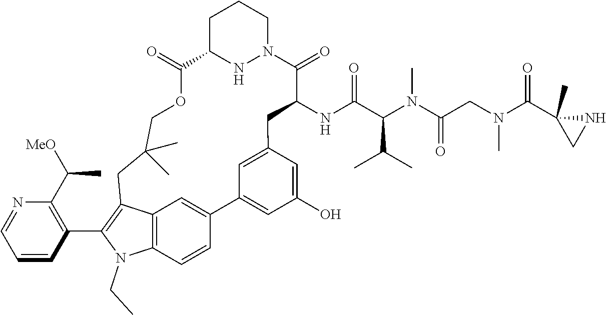 |
| 257 | 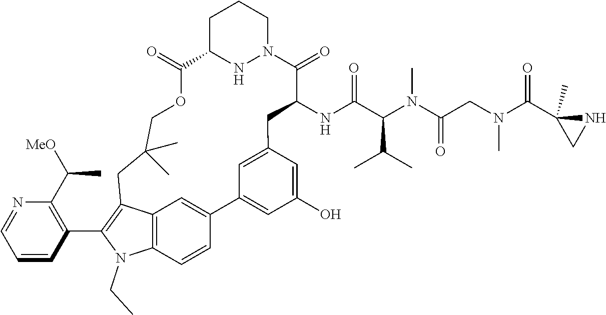 |
| 258* | 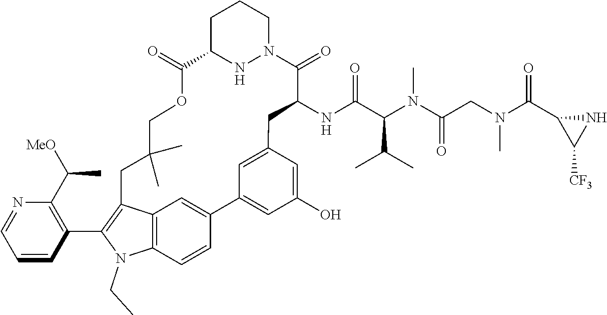 |
| 259 | 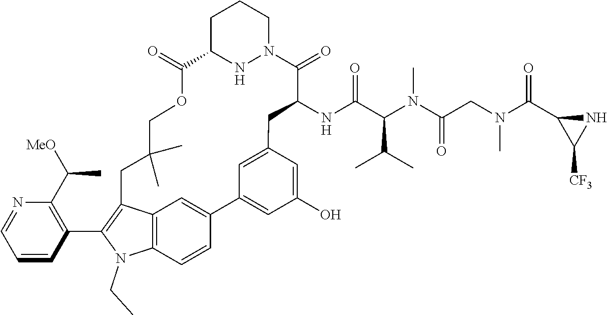 |
| 260* | 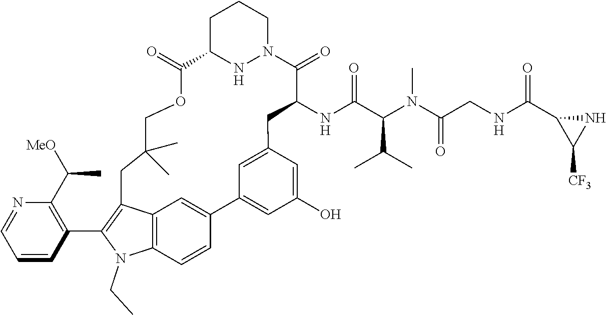 |
| 261* | 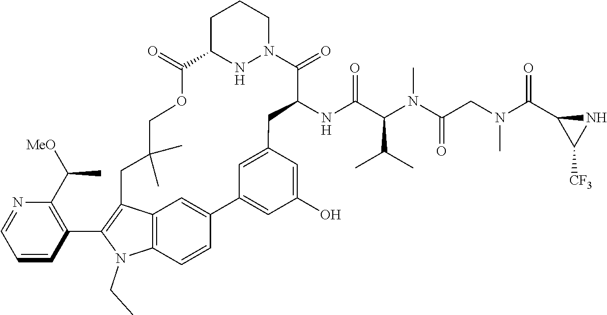 |
| 262 | 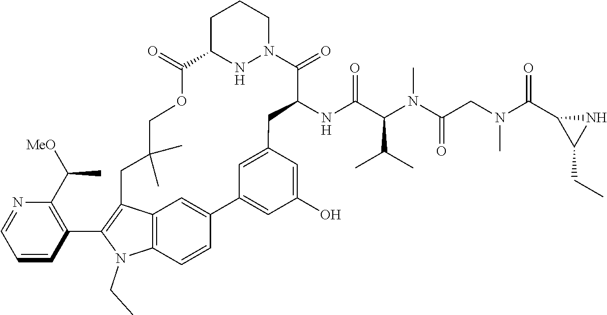 |
| 263 | 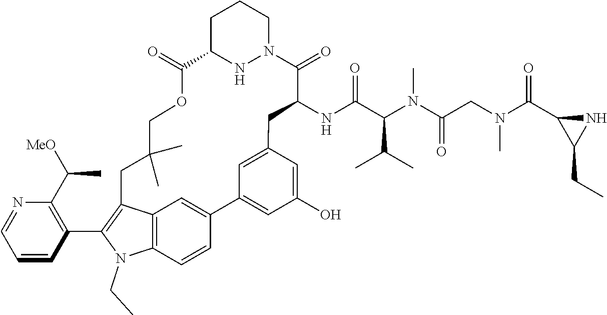 |
| 264 | 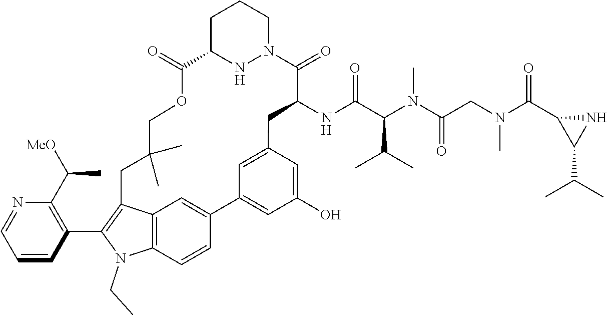 |
| 265 | 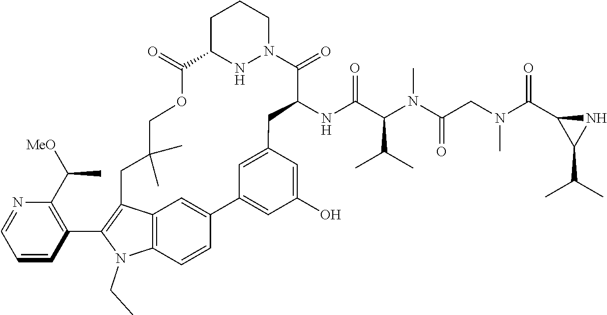 |
| 266 | 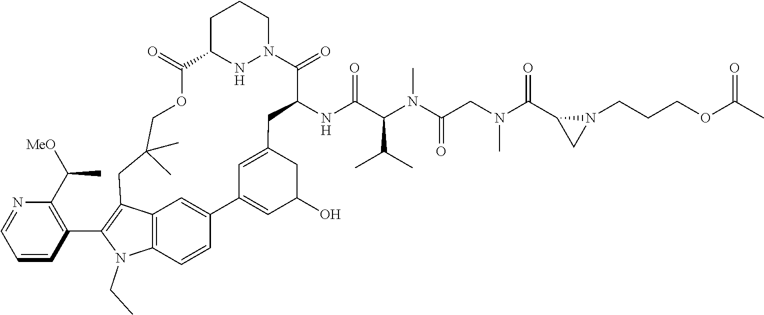 |
| 267 | 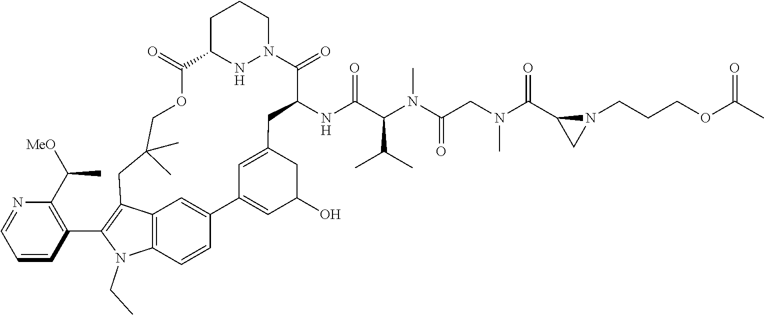 |
| 268 | 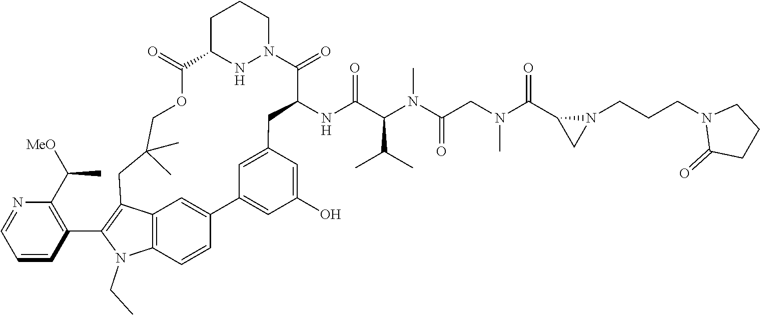 |
| 269 | 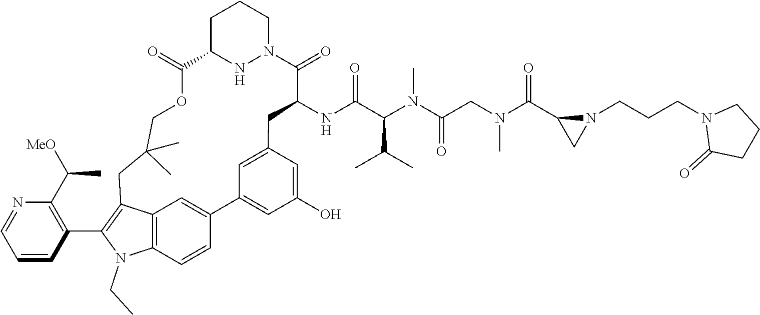 |
| 270 | 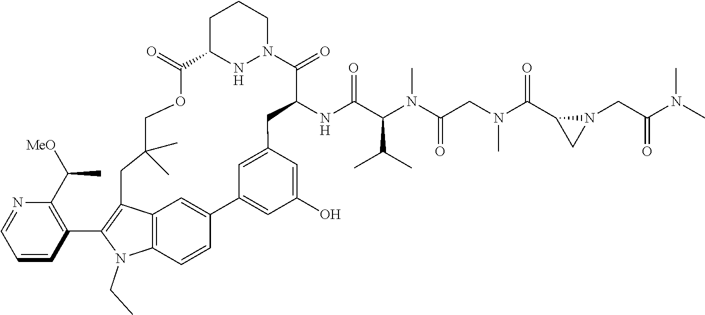 |
| 271 | 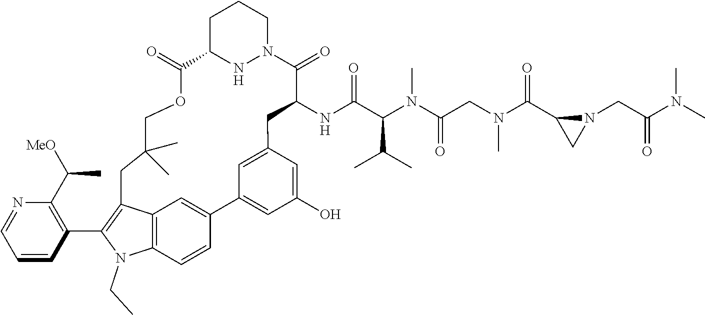 |
| 272 | 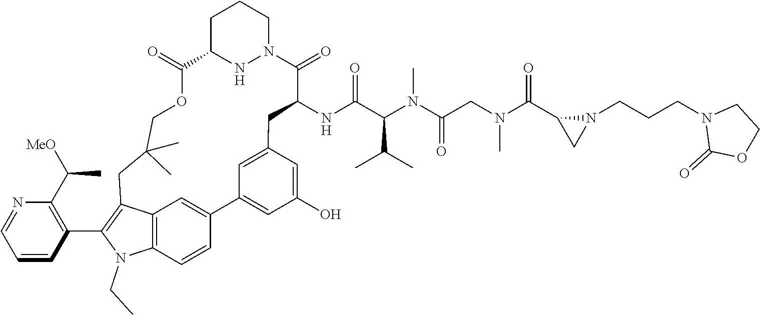 |
| 273 | 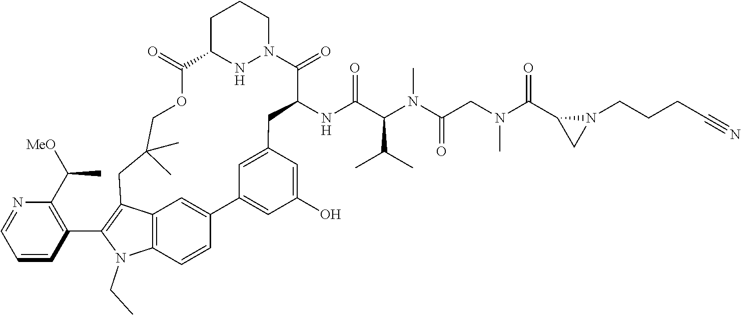 |
| 274 | 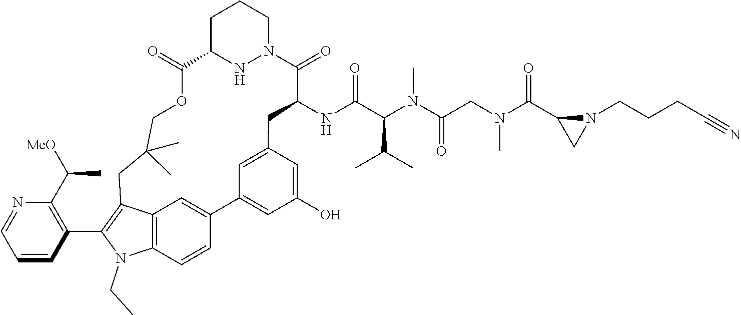 |
| 275 | 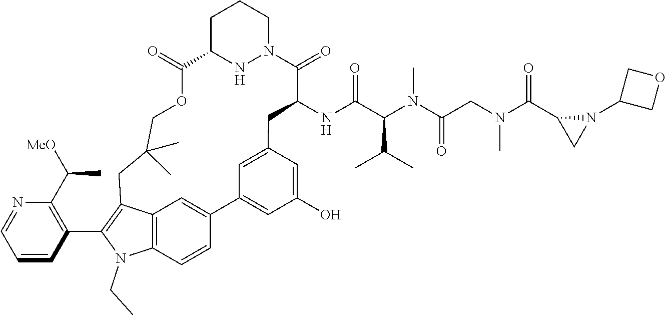 |
| 276 | 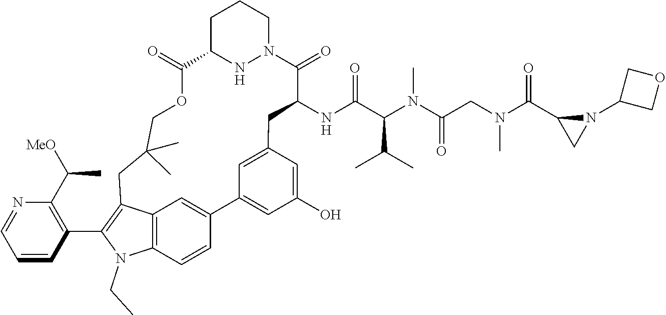 |
| 277 | 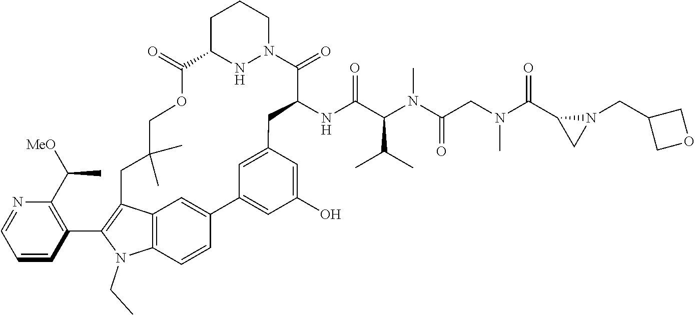 |
| 278 | 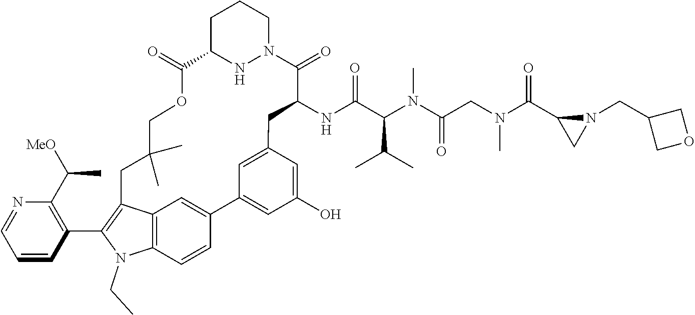 |
| 279 | 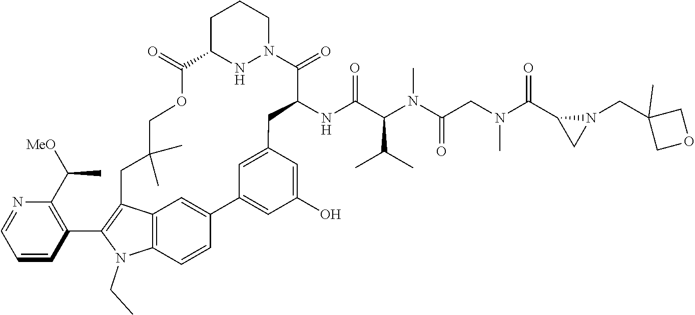 |
| 280 | 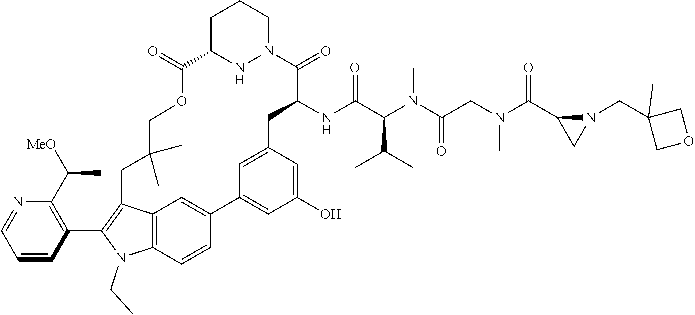 |
| 281* | 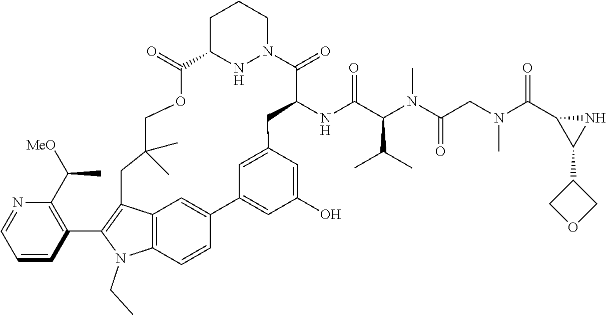 |
| 282* | 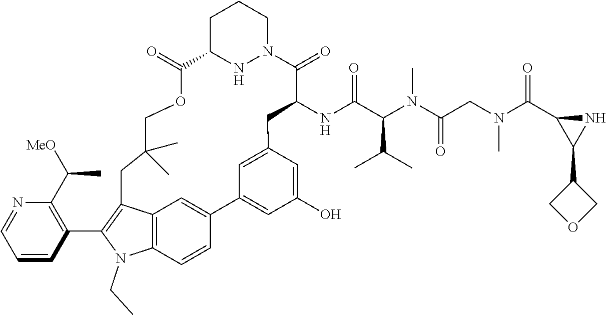 |
| 283* | 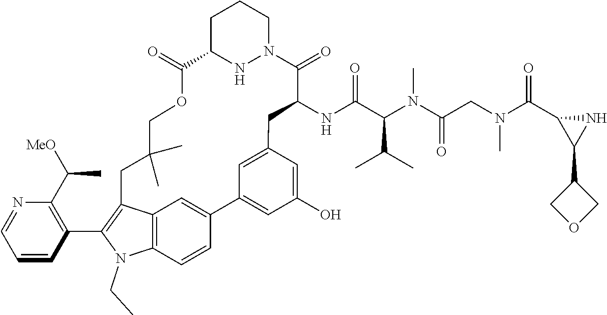 |
| 284* | 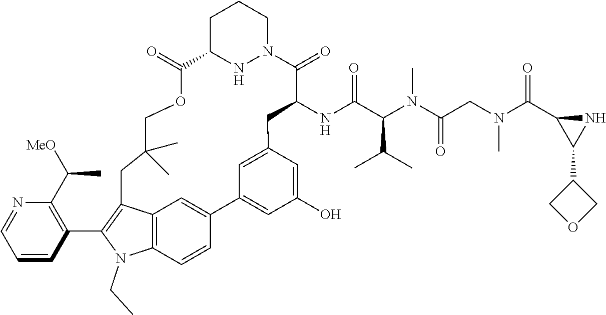 |
| 285 | 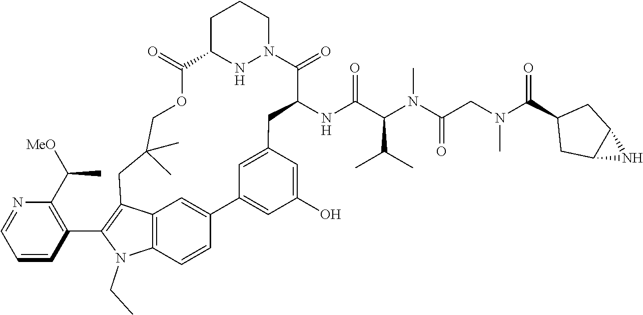 |
| 286 | 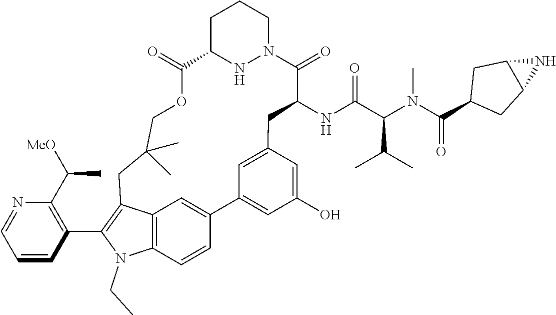 |
| 287 | 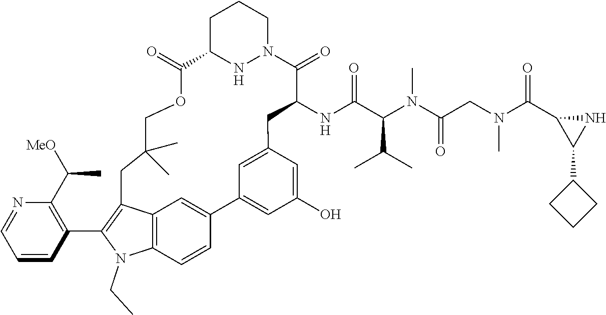 |
| 288 | 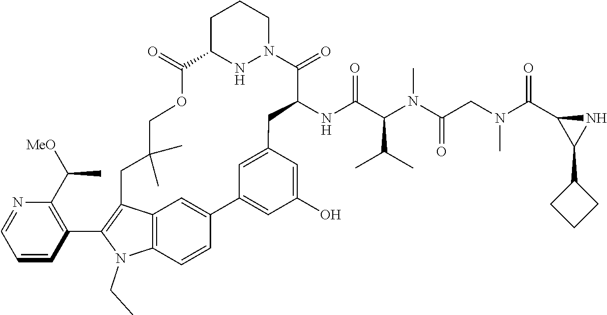 |
| 289 | 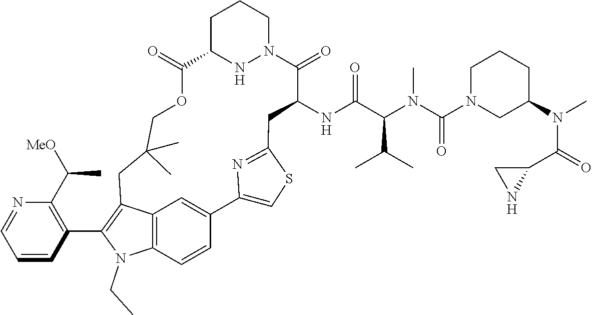 |
| 290 | 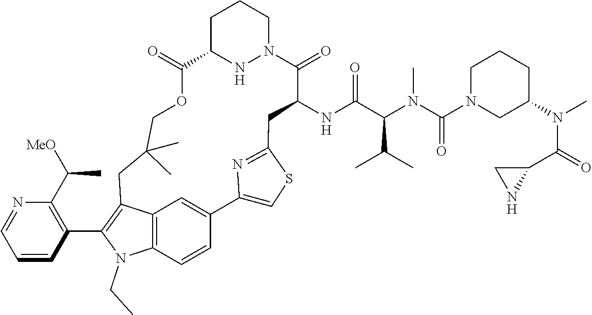 |
| 291 | 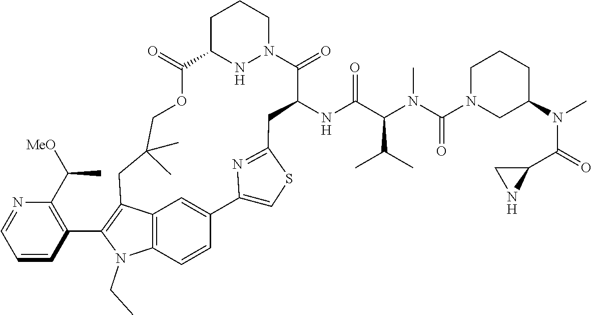 |
| 292 | 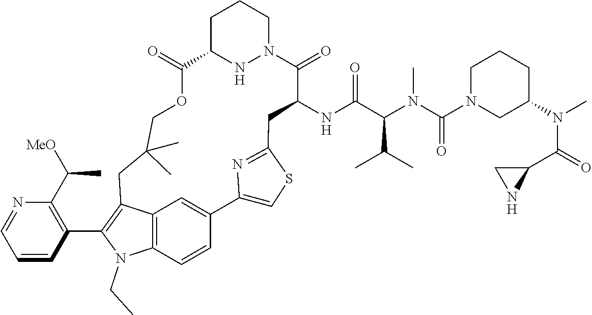 |
| 293 | 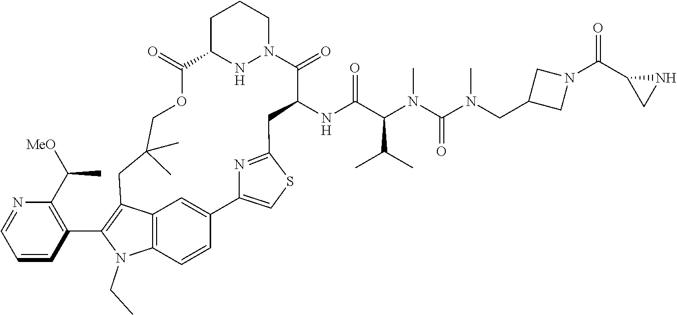 |
| 294 | 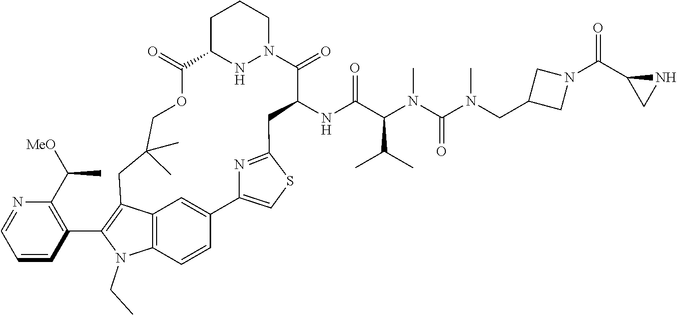 |
| 295 | 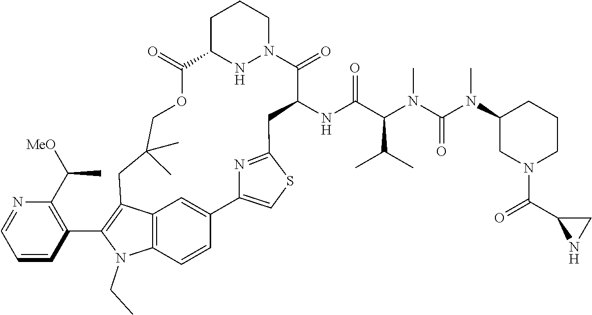 |
| 296 | 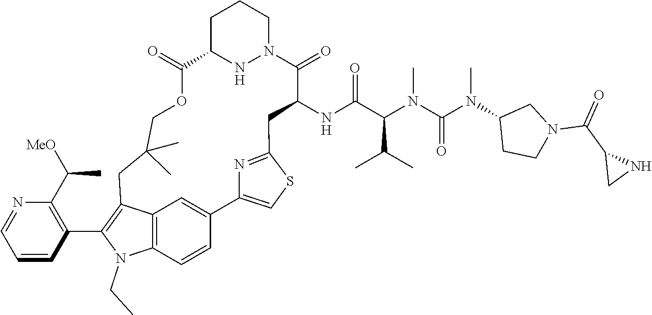 |
| 297 | 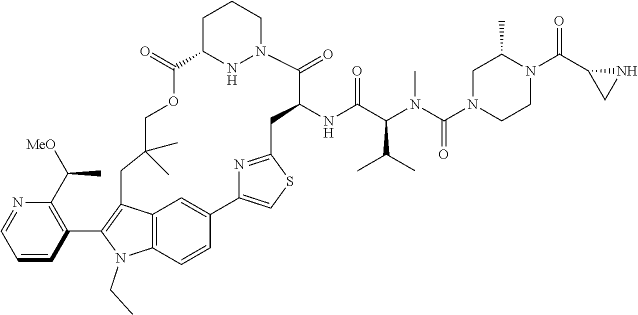 |
| 298 | 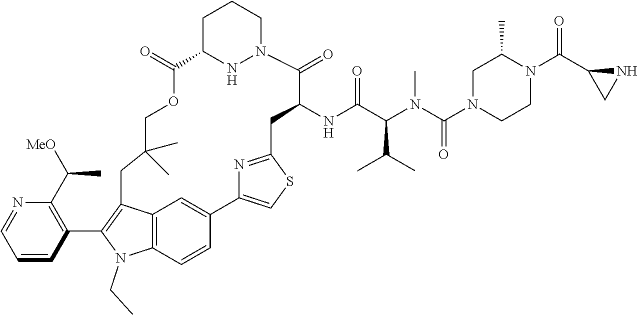 |
| 299 | 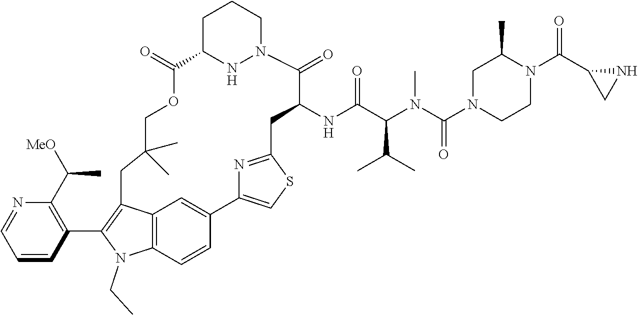 |
| 300 | 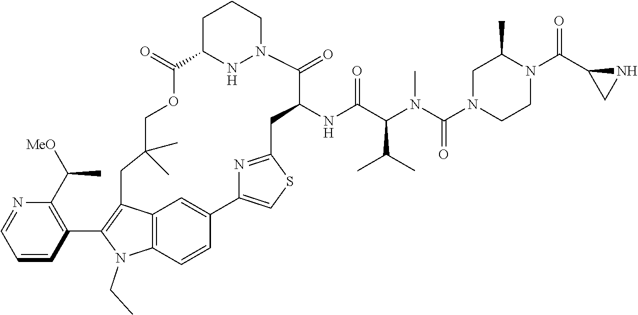 |
| 301 | 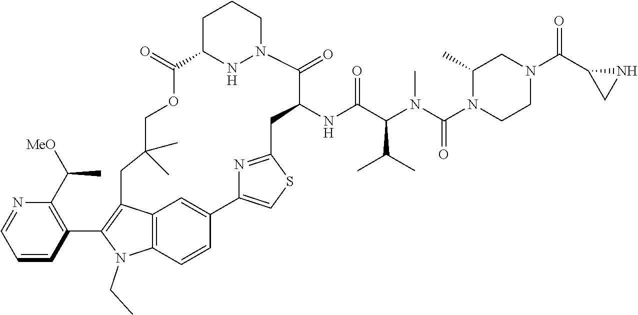 |
| 302 | 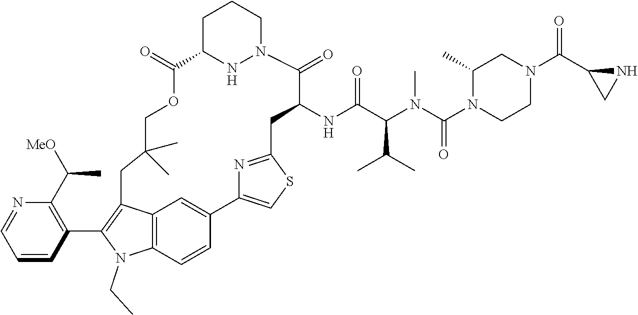 |
| 303 | 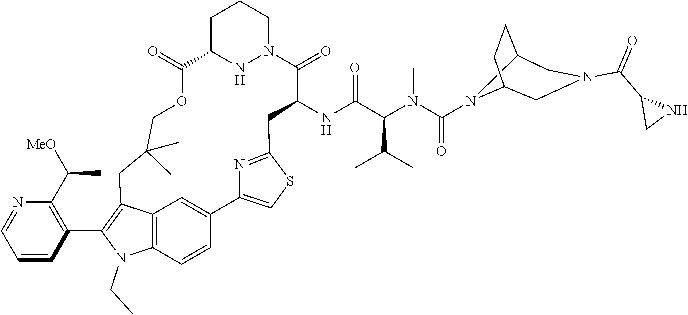 |
| 304 | 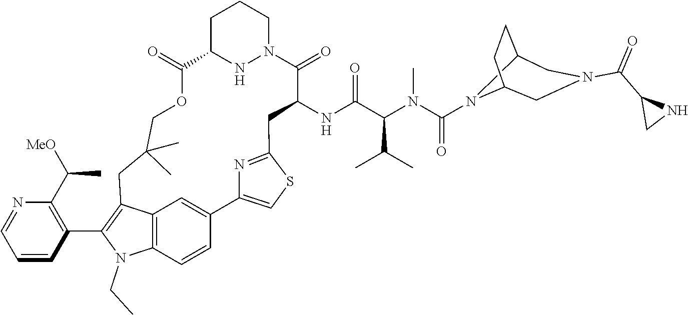 |
| 305 | 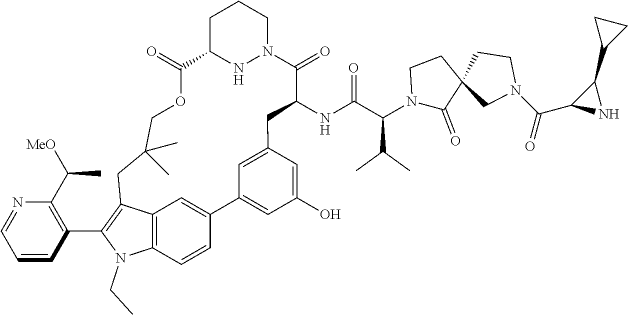 |
| 306 | 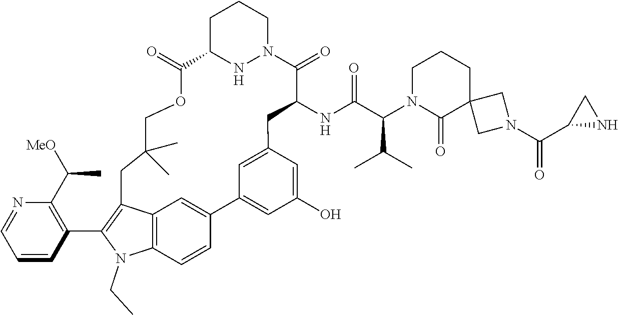 |
| 307 | 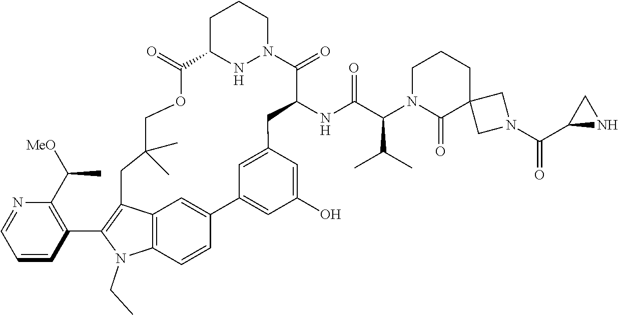 |
| 308* | 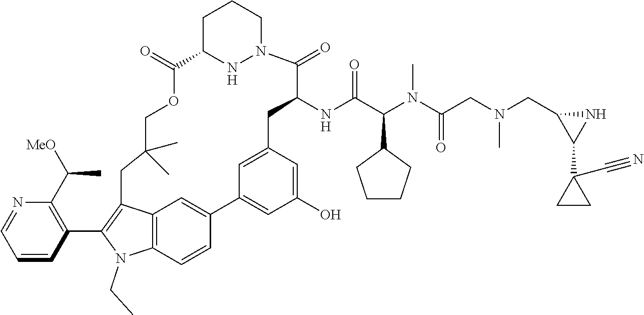 |
| 309 | 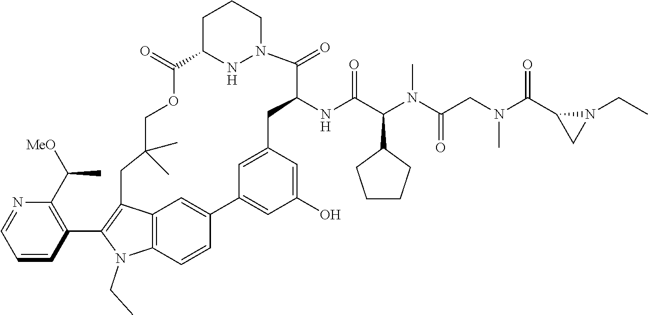 |
| 310 | 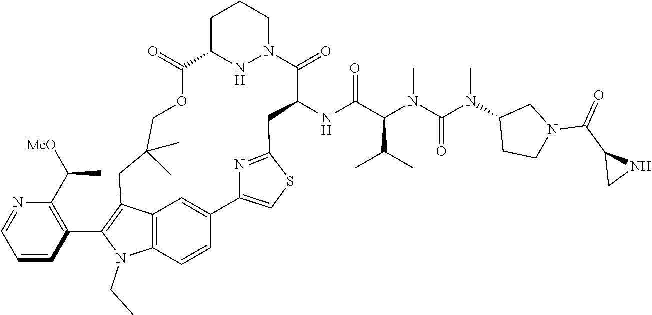 |
| 311 | 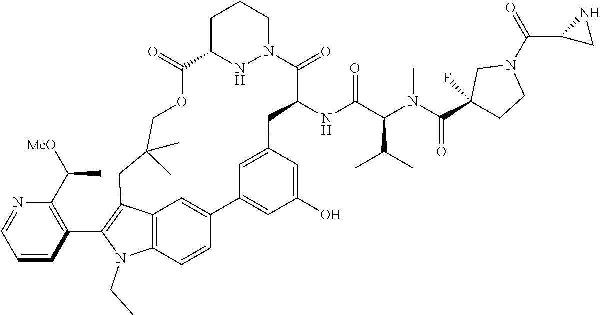 |
| 312 | 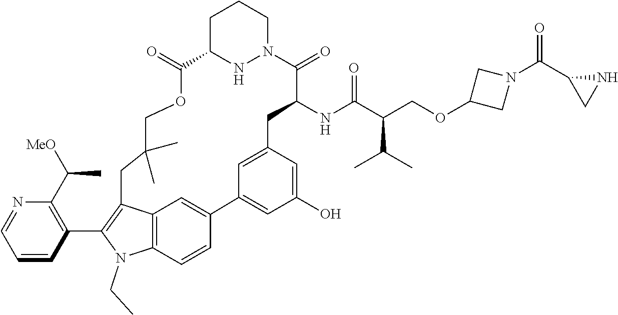 |
| 313 | 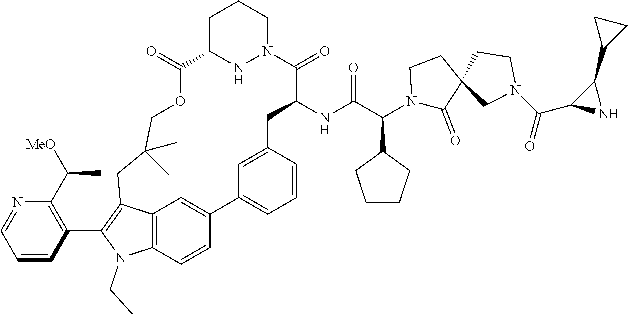 |
| 314* | 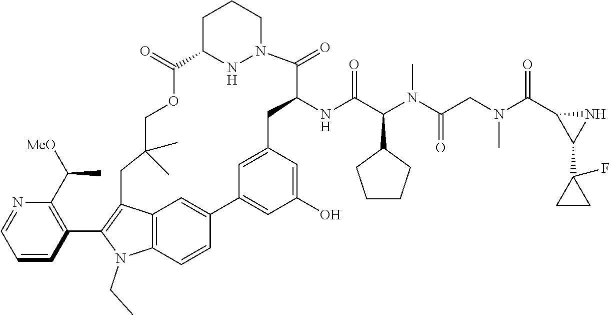 |
| 315* | 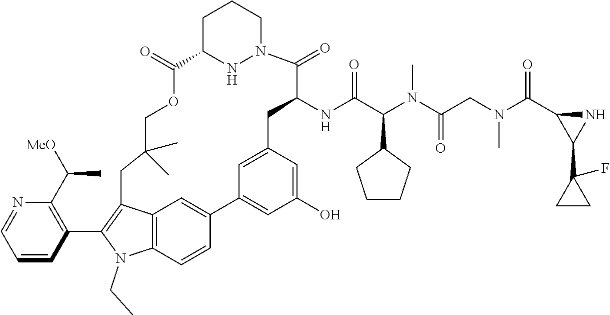 |
| 316* | 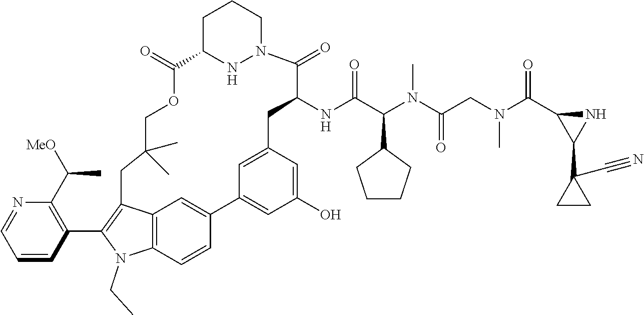 |
| 317 | 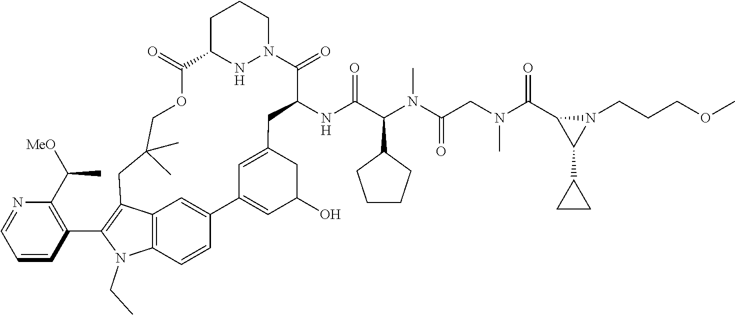 |
| 318 | 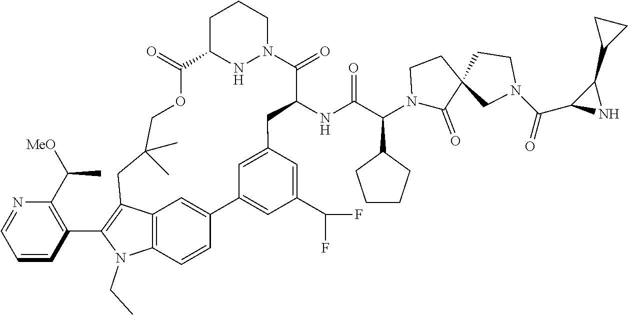 |
| 319 | 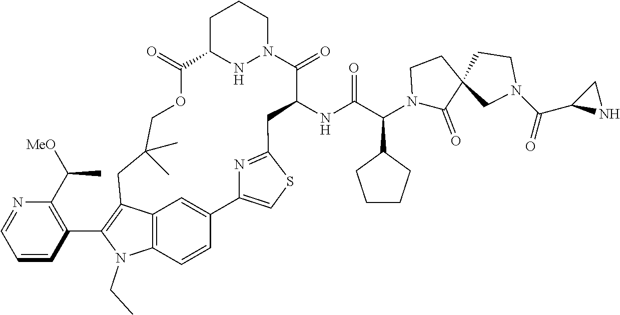 |
| 320 | 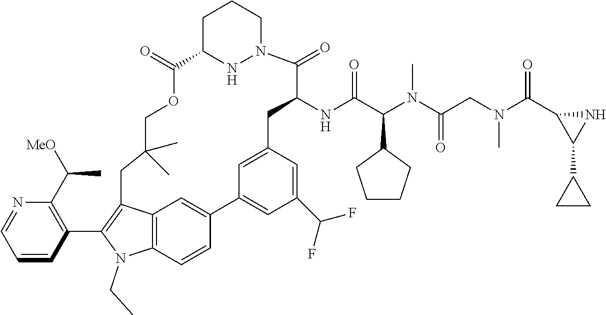 |
| 321 | 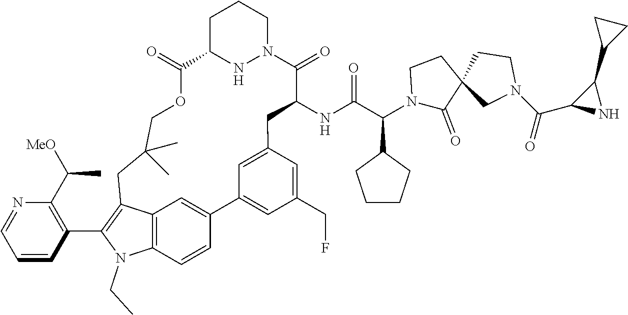 |
| 322* | 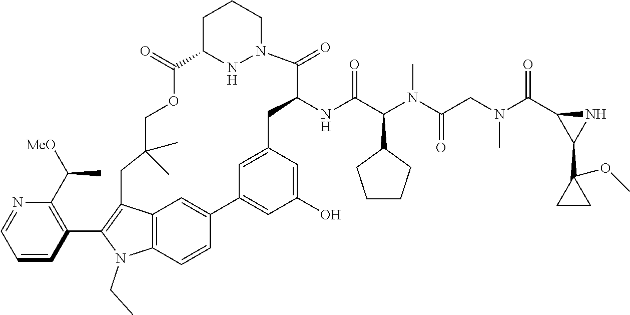 |
| 323* | 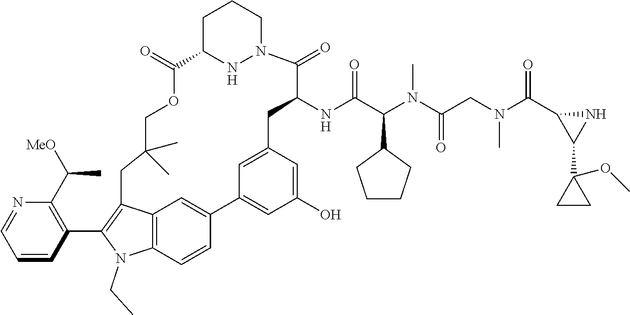 |
| 324 | 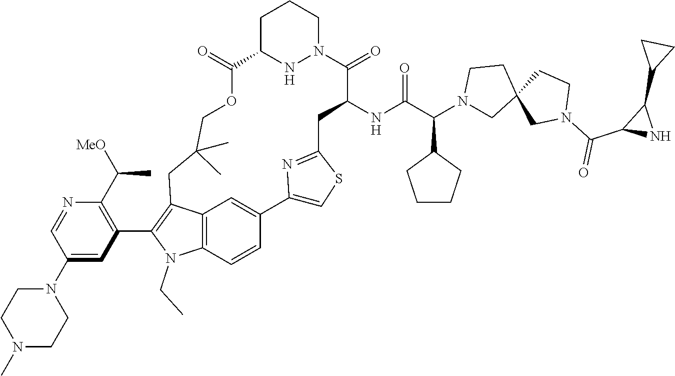 |
| 325 | 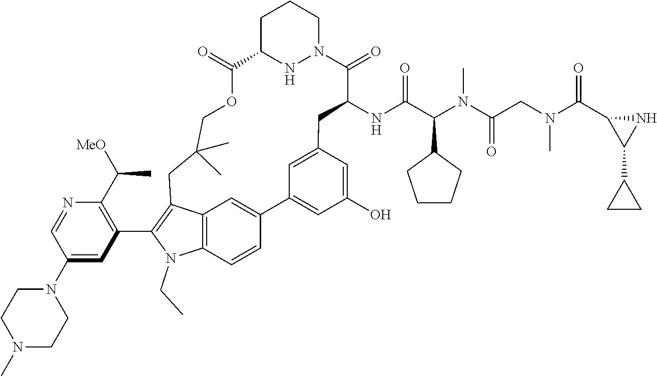 |
| 326 | 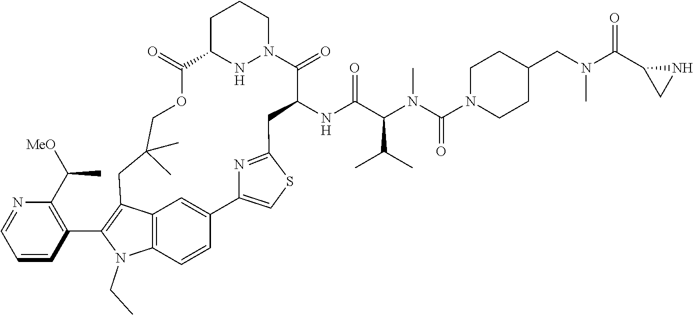 |
| 327 | 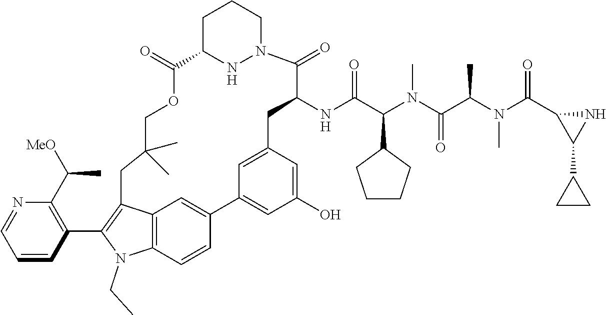 |
| 328 | 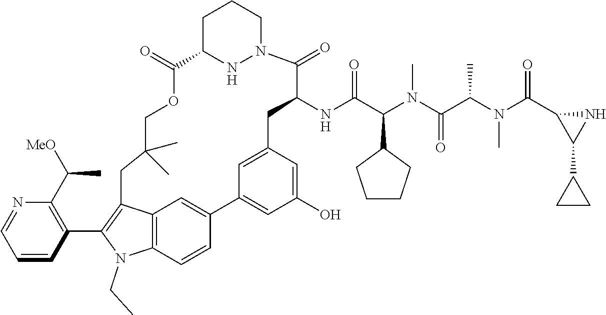 |
| 329 | 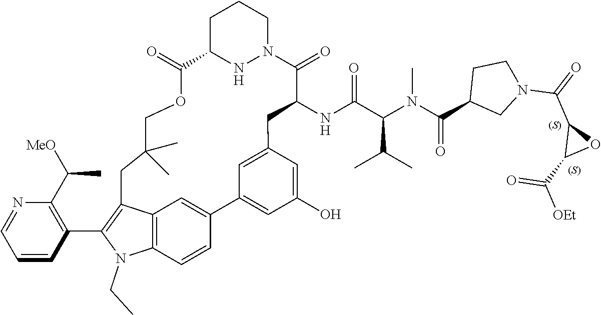 |
| 330 | 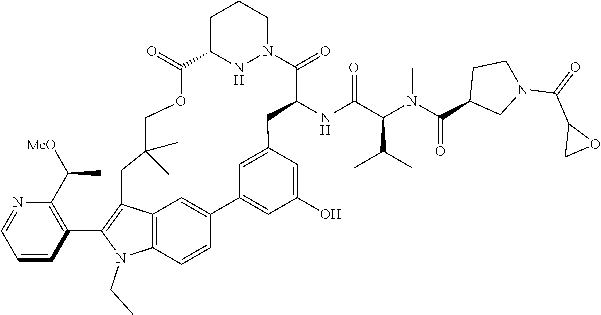 |
| 331 | 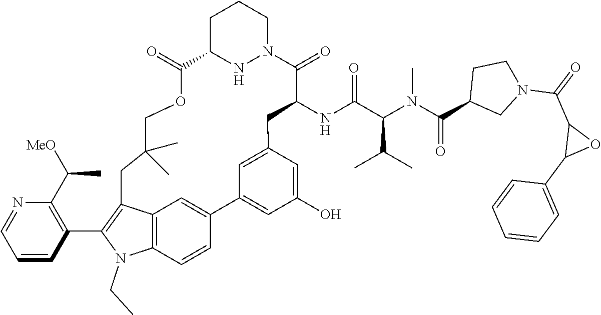 |
| 332 | 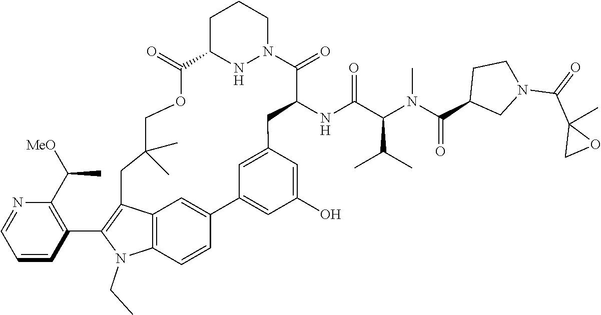 |

*Stereochemistry of the aziridine carbon is assumed.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, a compound of Table 2 is provided, or a pharmaceutically acceptable salt thereof. In some embodiments, a compound of the present invention is selected from Table 2, or a pharmaceutically acceptable salt or atropisomer thereof.

TABLE 2
Certain Compounds of the Present Invention

| Ex# | Structure |
|---|---|
| B5 | 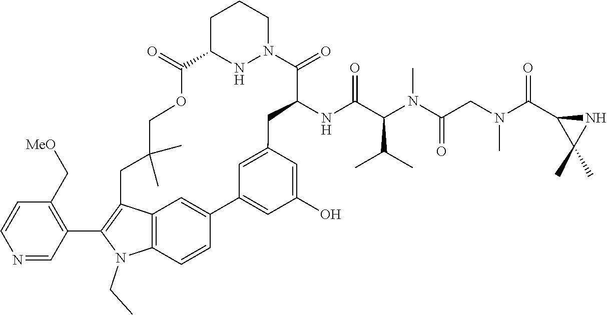 |
| B6 | 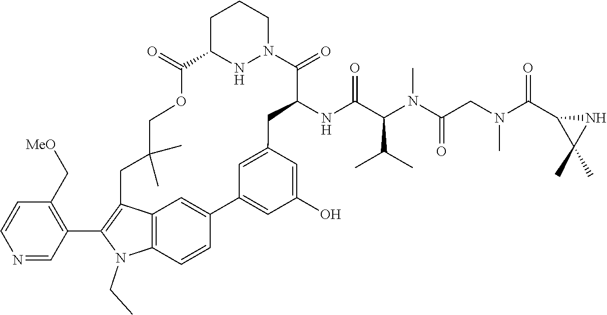 |
| B8 | 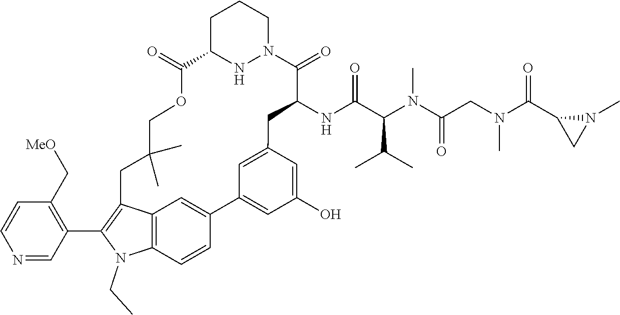 |
| B9 | 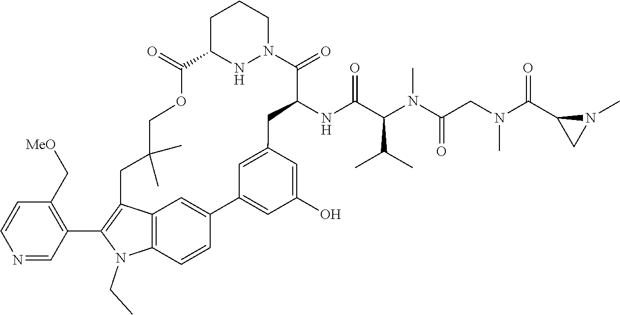 |
| B16 | 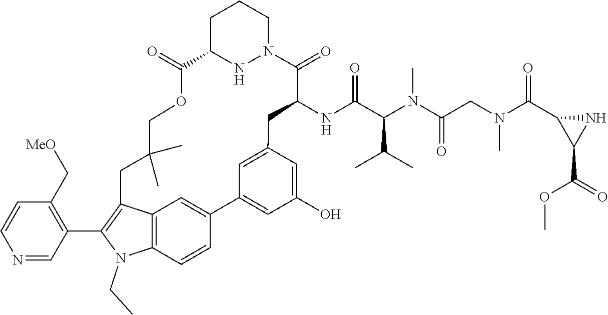 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C(=O)OC)C(=O)N1CCC[C@H](N1)C(=O)OCC(C)(C)C2] |
| B19 | 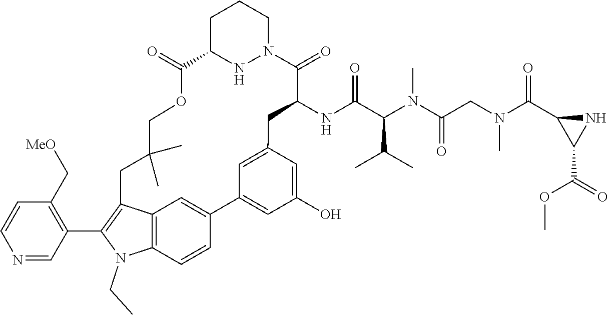 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C(=O)OC)C(=O)N1CCC[C@H](N1)C(=O)OCC(C)(C)C2] |
| B29 | 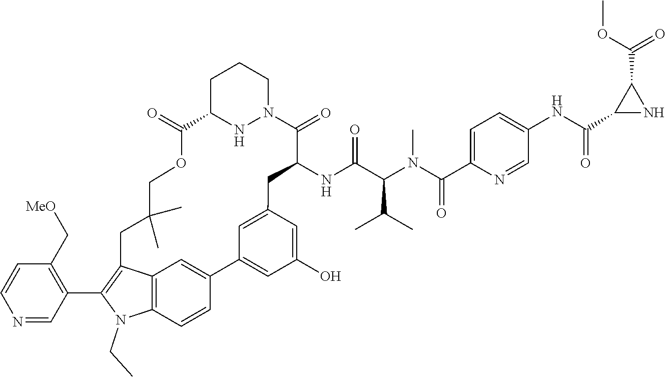 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@H]3C(=O)OC)cn1)C(=O)N1CCC[C@H](N1)C(=O)OCC(C)(C)C2] |
| B30 | 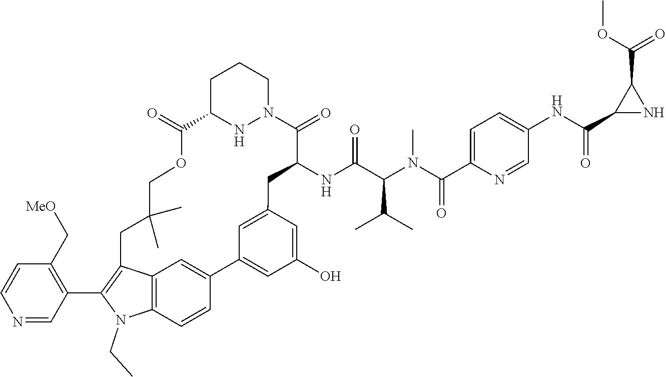 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@@H]3C(=O)OC)cn1)C(=O)N1CCC[C@H](N1)C(=O)OCC(C)(C)C2] |
| B31 | 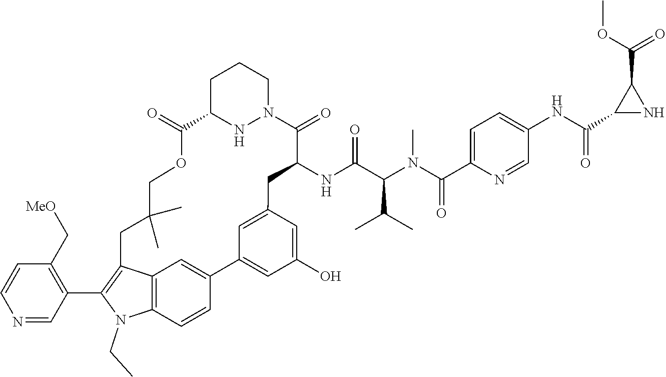 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@@H]3C(=O)OC)cn1)C(=O)N1CCC[C@H](N1)C(=O)OCC(C)(C)C2] |
| B32 | 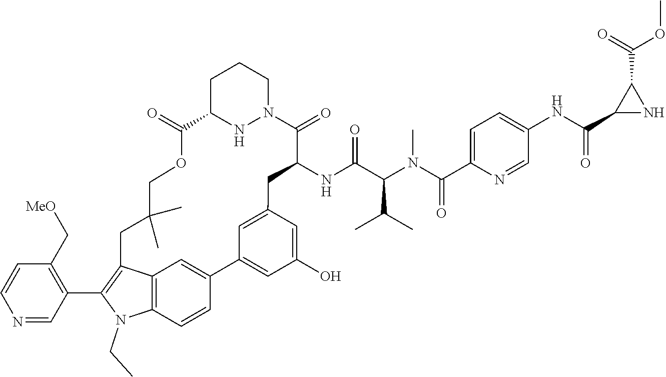 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@H]3C(=O)OC)cn1)C(=O)N1CCC[C@H](N1)C(=O)OCC(C)(C)C2] |
| B35 | 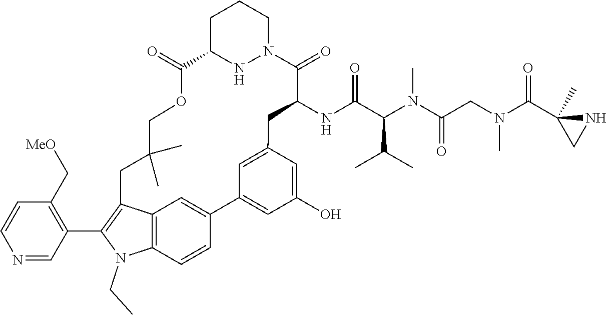 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@]1(C)CN1)C(=O)N1CCC[C@H](N1)C(=O)OCC(C)(C)C2] |
| B36 | 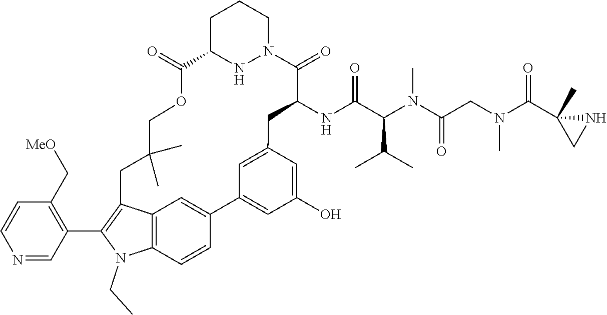 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@]1(C)CN1)C(=O)N1CCC[C@H](N1)C(=O)OCC(C)(C)C2] |
| B37 | 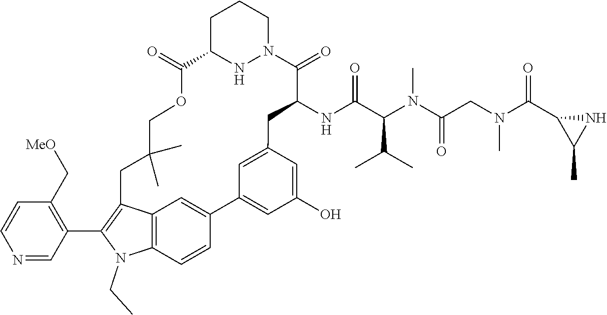 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C)C(=O)N1CCC[C@H](N1)C(=O)OCC(C)(C)C2] |
| B38 | 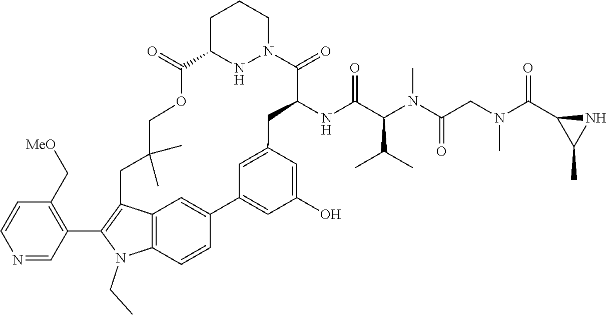 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C)C(=O)N1CCC[C@H](N1)C(=O)OCC(C)(C)C2] |
| B40 | 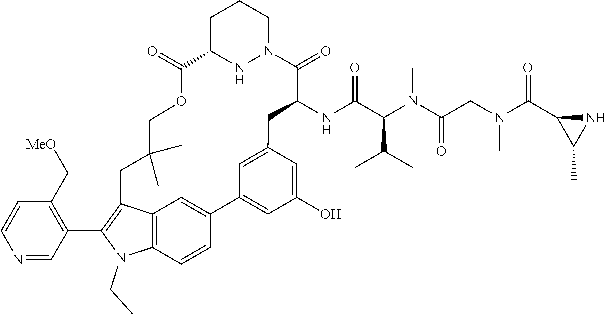 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C)C(=O)N1CCC[C@H](N1)C(=O)OCC(C)(C)C2] |
| B41 | 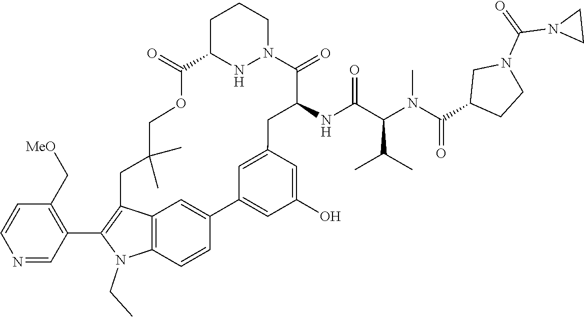 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3CC3)C1)C(=O)N1CCC[C@H](N1)C(=O)OCC(C)(C)C2] |
| B42 | 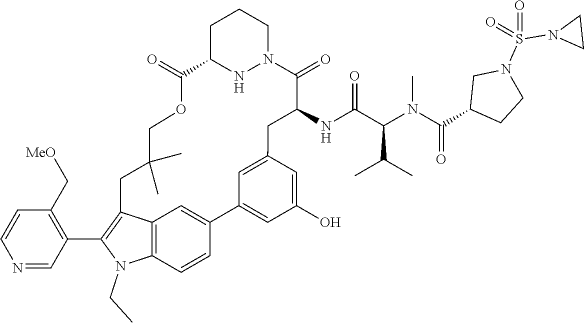 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3CC3)C1)C(=O)N1CCC[C@H](N1)C(=O)OCC(C)(C)C2] |
| B43 | 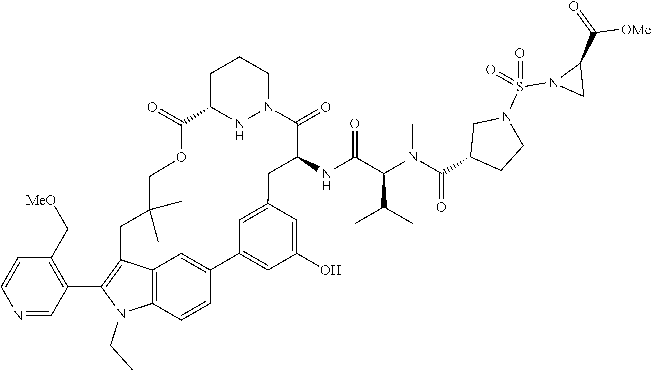 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@@H]3C(=O)OC)C1)C(=O)N1CCC[C@H](N1)C(=O)OCC(C)(C)C2] |
| B44 | 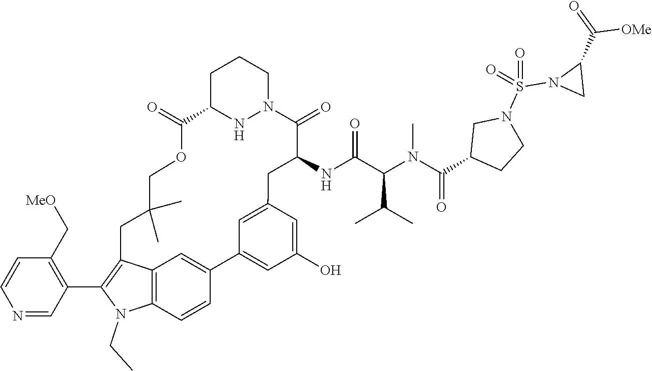 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@H]3C(=O)OC)C1)C(=O)N1CCC[C@H](N1)C(=O)OCC(C)(C)C2] |
| B46 | 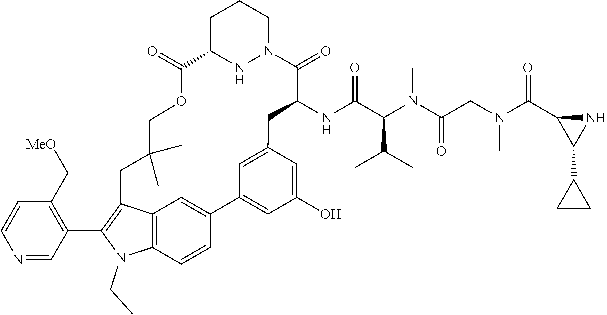 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C1CC1)C(=O)N1CCC[C@H](N1)C(=O)OCC(C)(C)C2] |
| B48 | 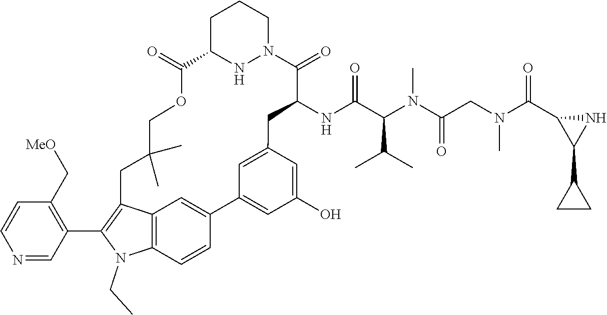 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C1CC1)C(=O)N1CCC[C@H](N1)C(=O)OCC(C)(C)C2] |
| B51 | 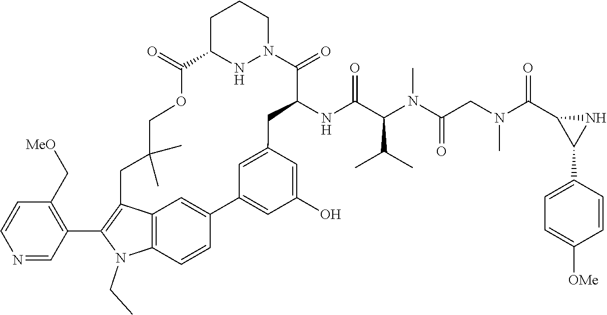 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| B53 | 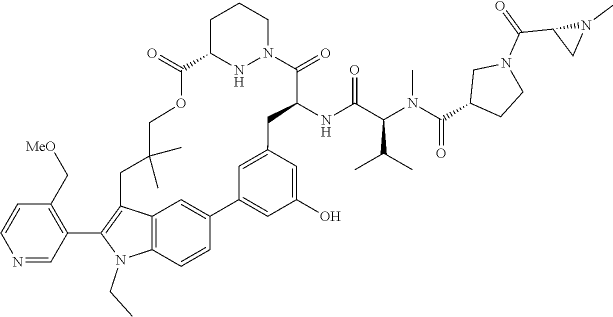 |
| B54 | 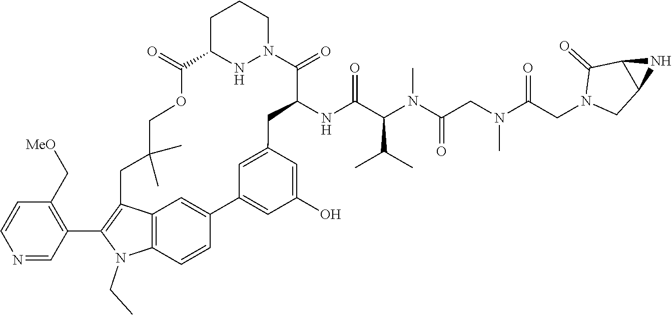 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@@H]3N[C@@H]3C1=O)C(=O)N1CCC[C@H](N1)C(=O)OCC(C)(C)C2] |
| B55 | 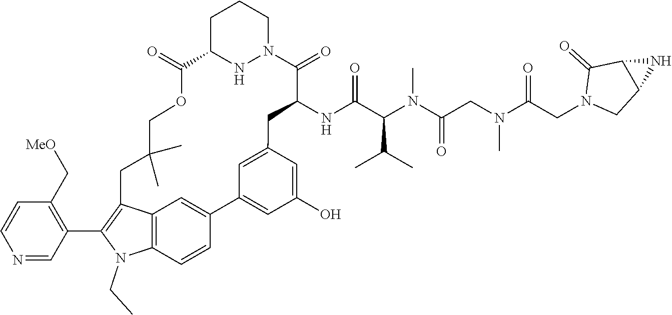 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@H]3N[C@H]3C1=O)C(=O)N1CCC[C@H](N1)C(=O)OCC(C)(C)C2] |
| B56 | 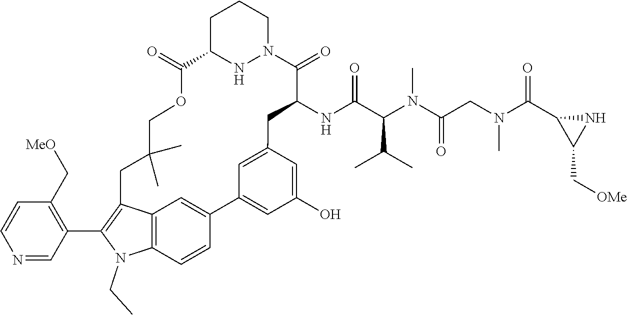 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1COC)C(=O)N1CCC[C@H](N1)C(=O)OCC(C)(C)C2] |
| B57 | 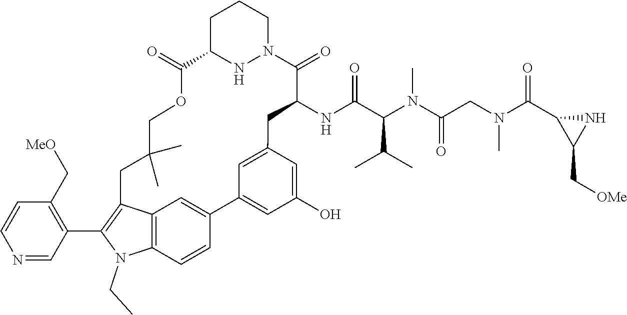 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1COC)C(=O)N1CCC[C@H](N1)C(=O)OCC(C)(C)C2] |
| B58 | 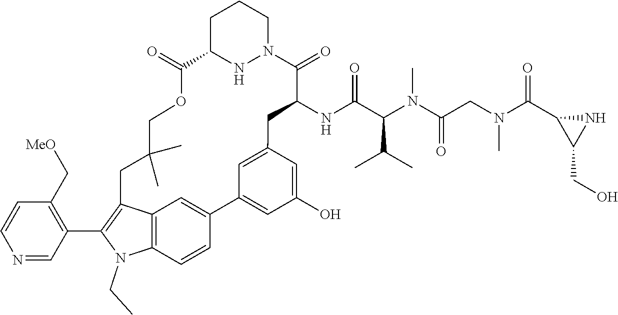 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1CO)C(=O)N1CCC[C@H](N1)C(=O)OCC(C)(C)C2] |
| B59 | 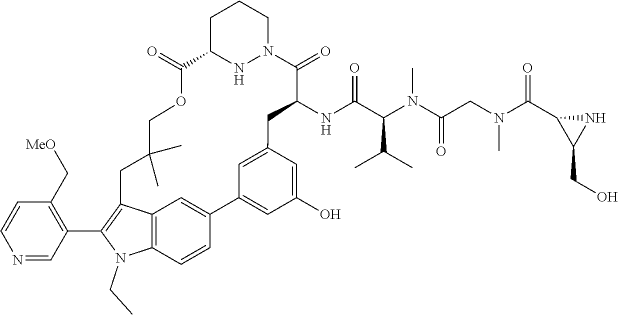 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1CO)C(=O)N1CCC[C@H](N1)C(=O)OCC(C)(C)C2] |
| B60 | 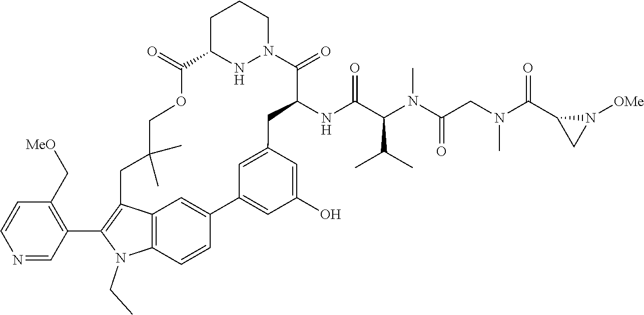 |
| B61 | 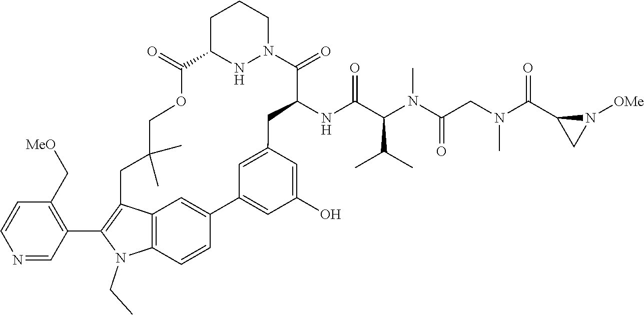 |
| B62 | 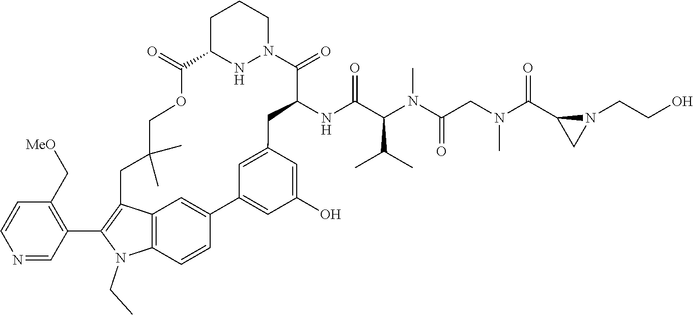 |
| B63 | 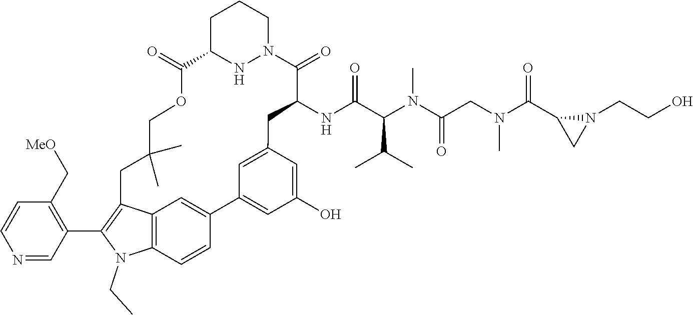 |
| B73 | 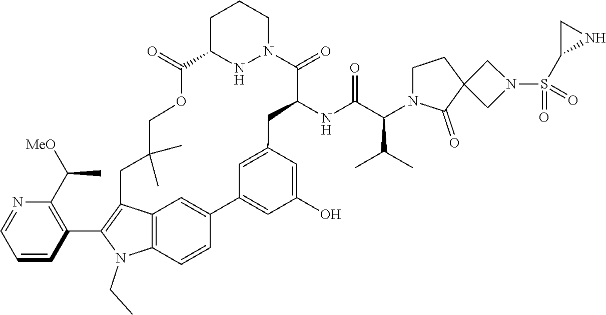 |
| B74 | 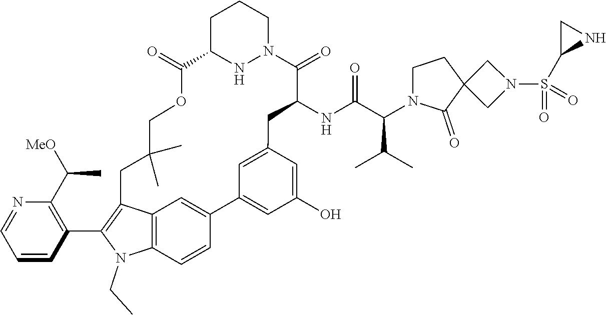 |
| B76 | 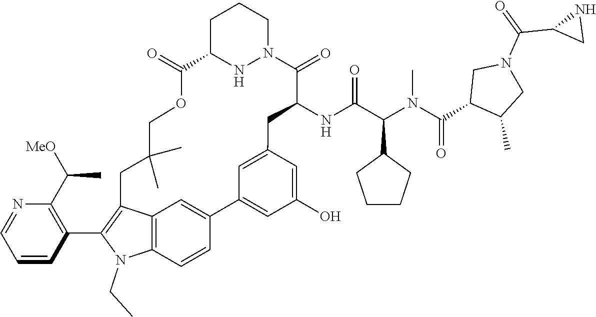 |
| B78 | 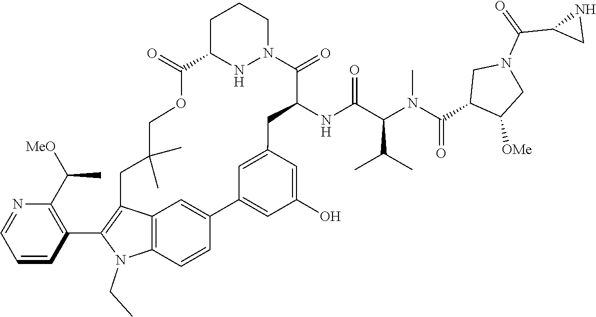 |
| B79 | 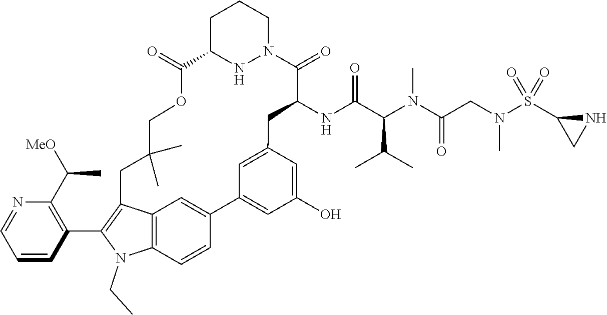 |
| B80 | 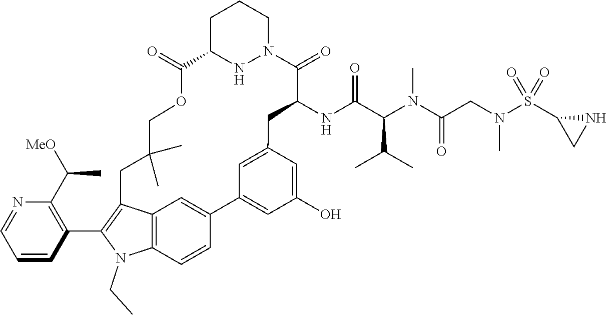 |
| B83 | 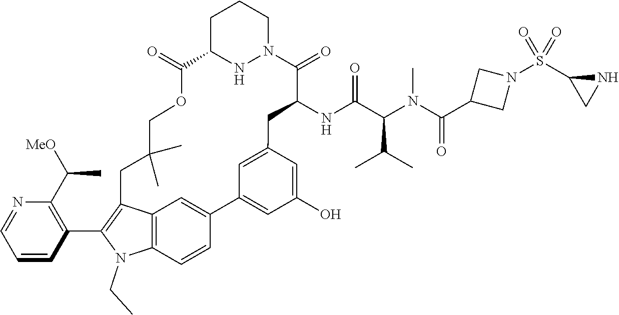 |
| B84 | 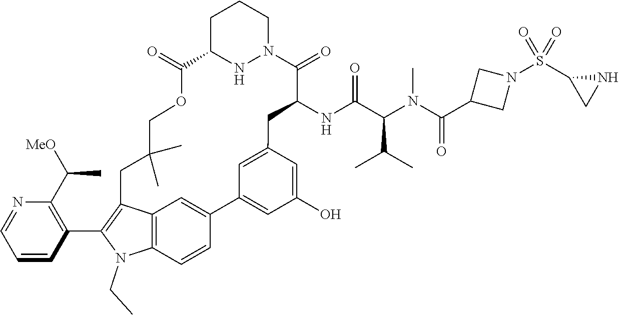 |
| B87 | 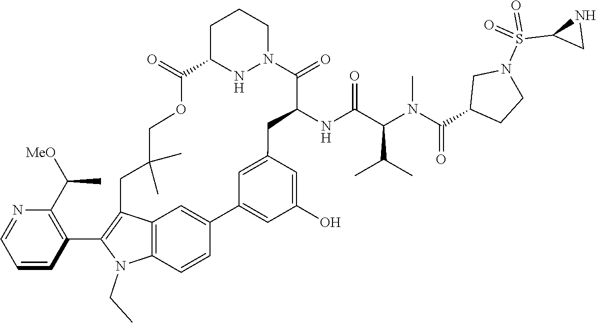 |
| B88 | 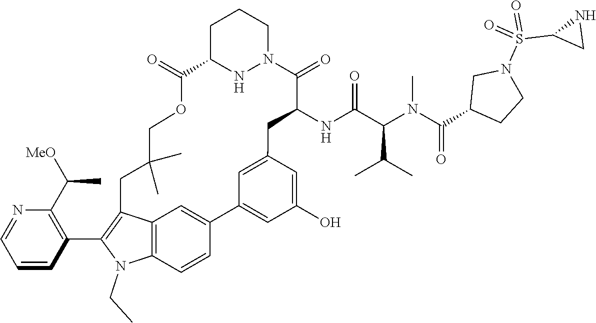 |
| B91 | 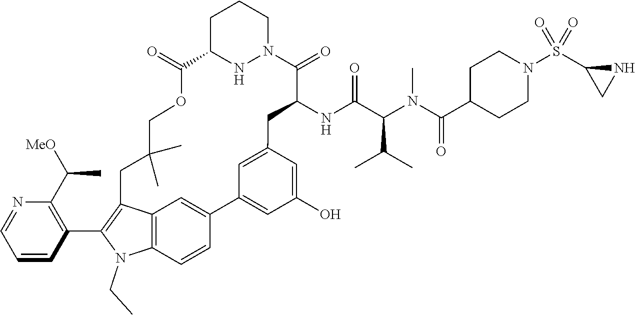 |
| B92 | 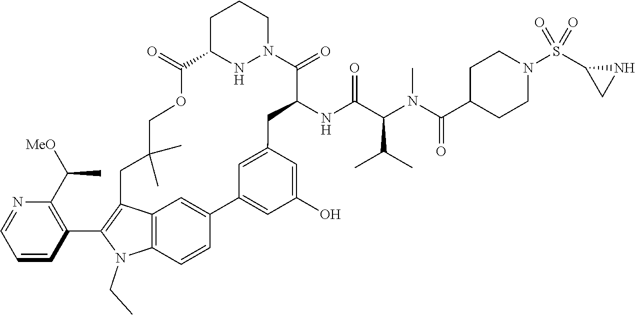 |
| B97 | 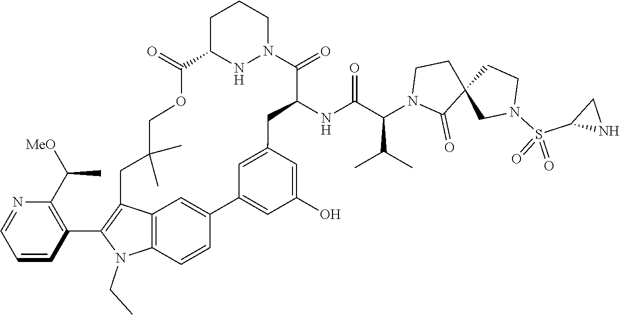 |
| B98 | 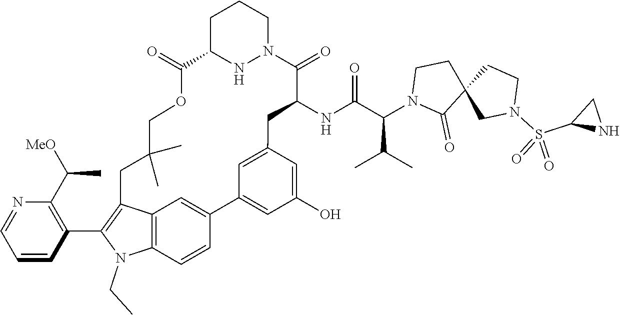 |
| B101 | 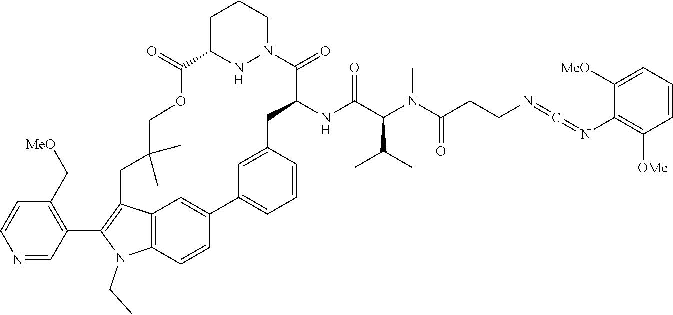 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cccc1OC)C(=O)N1CCC[C@H](N1)C(=O)OCC(C)(C)C2] |
| B108 | 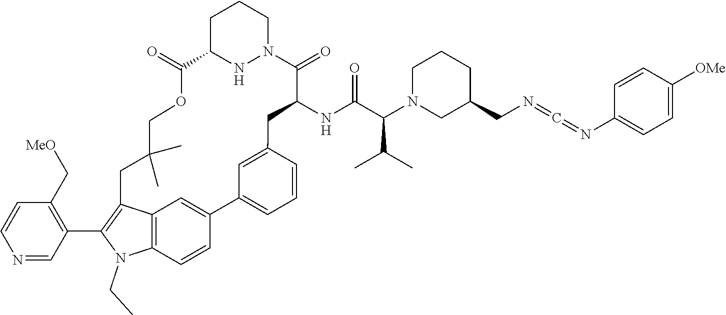 [SMILES: CCn1c(-c2cnccc2COC)c2c3cc(ccc31)-c1cccc(c1)C[C@H](NC(=O)[C@H](C(C)C)N1CCC[C@@H](CN=C=Nc3ccc(OC)cc3)C1)C(=O)N1CCC[C@H](N1)C(=O)OCC(C)(C)C2] |
| B109 | 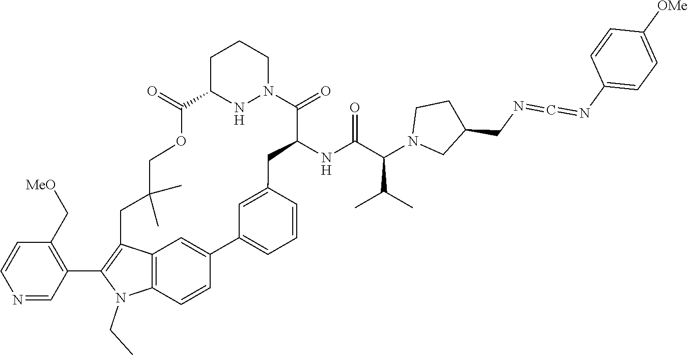 [SMILES: CCn1c(-c2cnccc2COC)c2c3cc(ccc31)-c1cccc(c1)C[C@H](NC(=O)[C@H](C(C)C)N1CC[C@@H](CN=C=Nc3ccc(OC)cc3)C1)C(=O)N1CCC[C@H](N1)C(=O)OCC(C)(C)C2] |
| B113 | 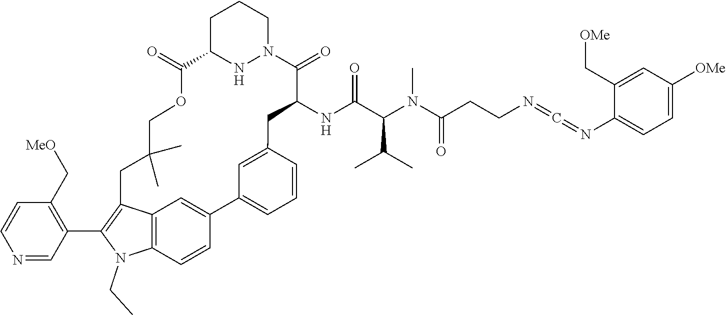 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1COC)C(=O)N1CCC[C@H](N1)C(=O)OCC(C)(C)C2] |
| B116 | 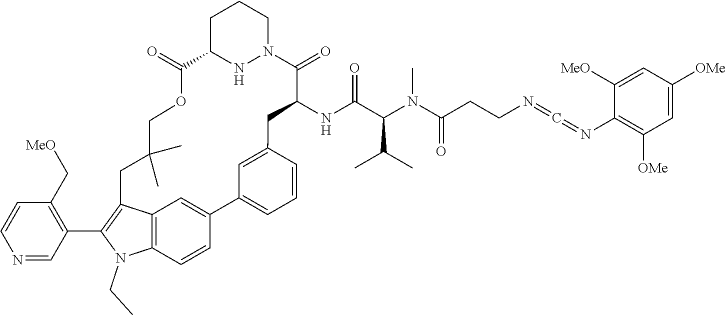 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cc(OC)cc1OC)C(=O)N1CCC[C@H](N1)C(=O)OCC(C)(C)C2] |
| B117 | 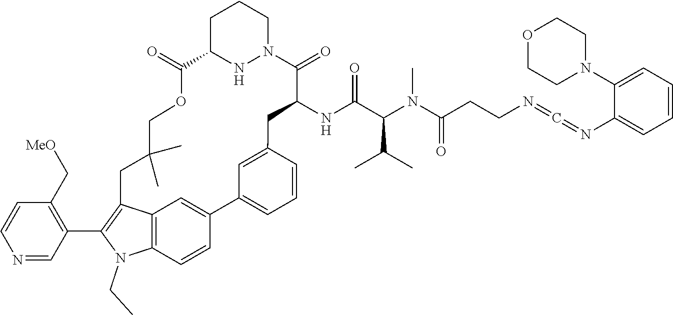 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1N1CCOCC1)C(=O)N1CCC[C@H](N1)C(=O)OCC(C)(C)C2] |
| B118 | 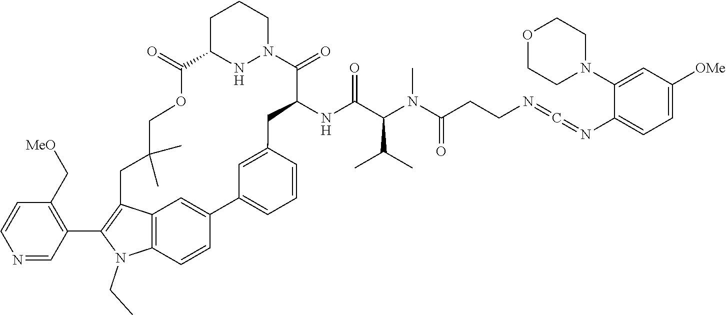 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1N1CCOCC1)C(=O)N1CCC[C@H](N1)C(=O)OCC(C)(C)C2] |
| B119 | 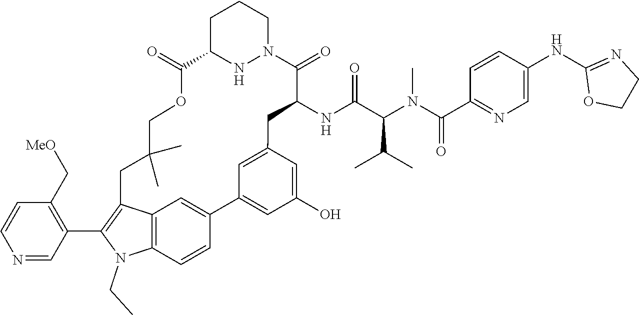 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC3=NCCO3)cn1)C(=O)N1CCC[C@H](N1)C(=O)OCC(C)(C)C2] |
| B120 | 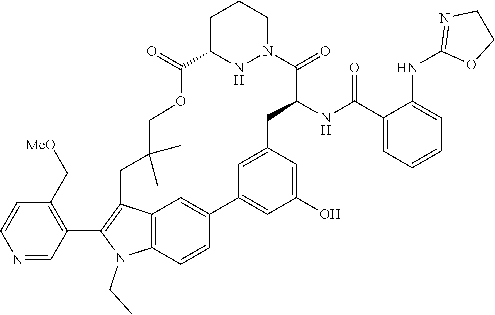 [SMILES: CCn1c(-c2cnccc2COC)c2c3cc(ccc31)-c1cc(O)cc(c1)C[C@H](NC(=O)c1ccccc1NC1=NCCO1)C(=O)N1CCC[C@H](N1)C(=O)OCC(C)(C)C2] |
| B122 | 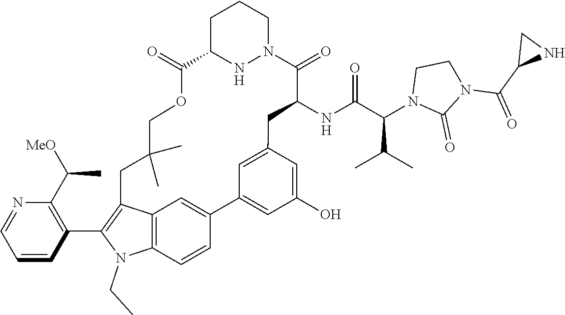 |
| B123 | 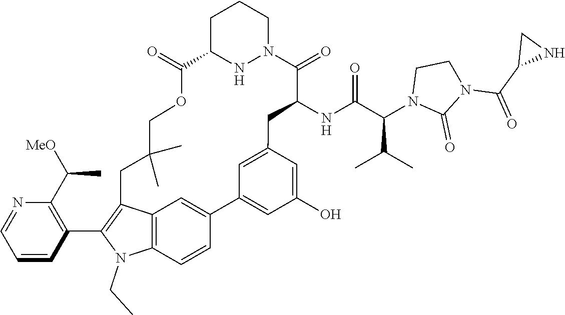 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Further provided is a conjugate, or salt thereof, comprising the structure of Formula IV:

M-L-P Formula IV
wherein L is a linker;
P is a monovalent organic moiety; and
M has the structure of Formula Va:

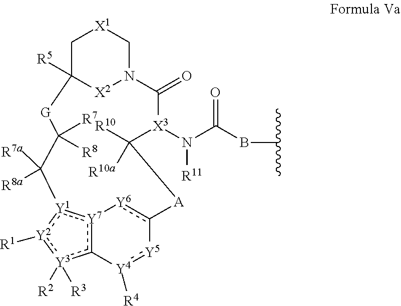

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O) NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R& combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula IV:

M-L-P Formula IV
wherein L is a linker;
P is a monovalent organic moiety; and
M has the structure of Formula Vb:

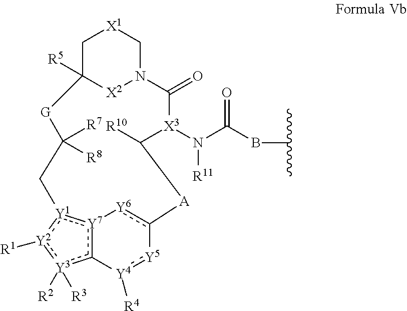

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the conjugate has the structure of Formula IV:

M-L-P Formula IV
wherein L is a linker;
P is a monovalent organic moiety; and
M has the structure of Formula Vc:

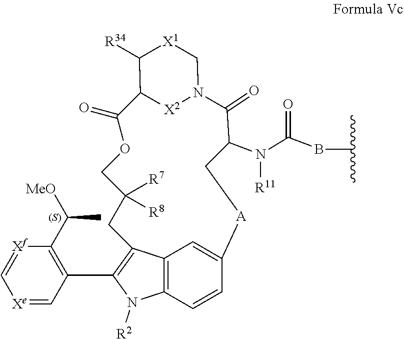

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
X^{e} and X^{f} are, independently, N or CH;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹¹ is hydrogen or C₁-C₃ alkyl; and
R³⁴ is hydrogen or C₁-C₃ alkyl.

In some embodiments of a compound of the present invention, X^{e} is N and X^{f} is CH. In some embodiments, X^{e} is CH and X^{f} is N.

In some embodiments, the conjugate has the structure of Formula IV:

M-L-P Formula IV
wherein L is a linker;
P is a monovalent organic moiety; and
M has the structure of Formula Vd:

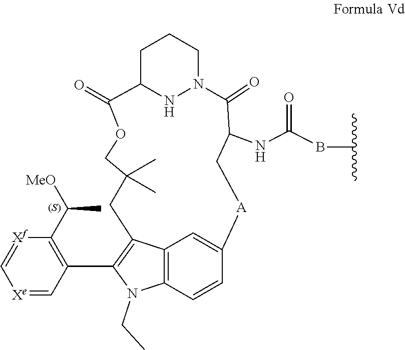

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of a compound of the present invention, X^{e} is N and X^{f} is CH. In some embodiments, X^{e} is CH and X^{f} is N.

In some embodiments of conjugates of the present invention, the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ- to —(B³)ᵢ—(C²)ⱼ—(B⁴)ₖ-A². In some embodiments of conjugates of the present invention, the linker is bound to the monovalent organic moiety through a bond to a carboxyl group of an amino acid residue of the monovalent organic moiety.

In some embodiments of conjugates of the present invention, the monovalent organic moiety is a protein. In some embodiments, the protein is a Ras protein. In some embodiments, the Ras protein is K-Ras G12D or K-Ras G13D.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Table 1 herein were prepared using methods disclosed herein or were prepared using methods disclosed herein combined with the knowledge of one of skill in the art.

Compounds of Table 2 may be prepared using methods disclosed herein or may be prepared using methods disclosed herein combined with the knowledge of one of skill in the art.

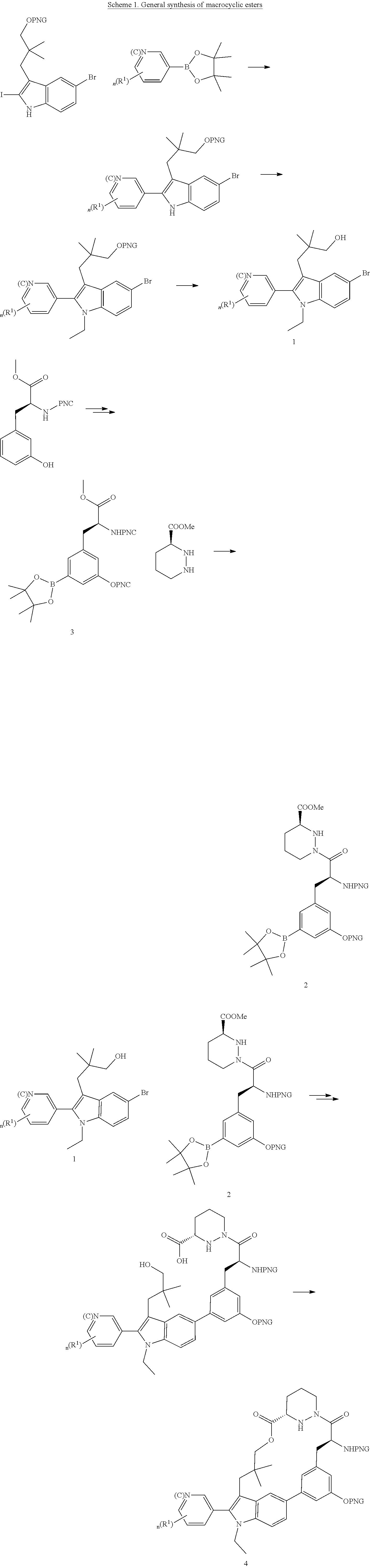

A general synthesis of macrocyclic esters is outlined in Scheme 1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl(S)-hexahydropyridazine-3-carboxylate.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of Pd catalyst followed by hydrolysis and macrolactonization steps to result in an appropriately protected macrocyclic intermediate (4). Additional deprotection or functionalization steps are required to produce a final compound. For example, a person of skill in the art would be able to install into a macrocyclic ester a desired-B-L-W group of a compound of Formula (I), where B, L and W are defined herein, including by using methods exemplified in certain Schemes below and in the Example section herein.

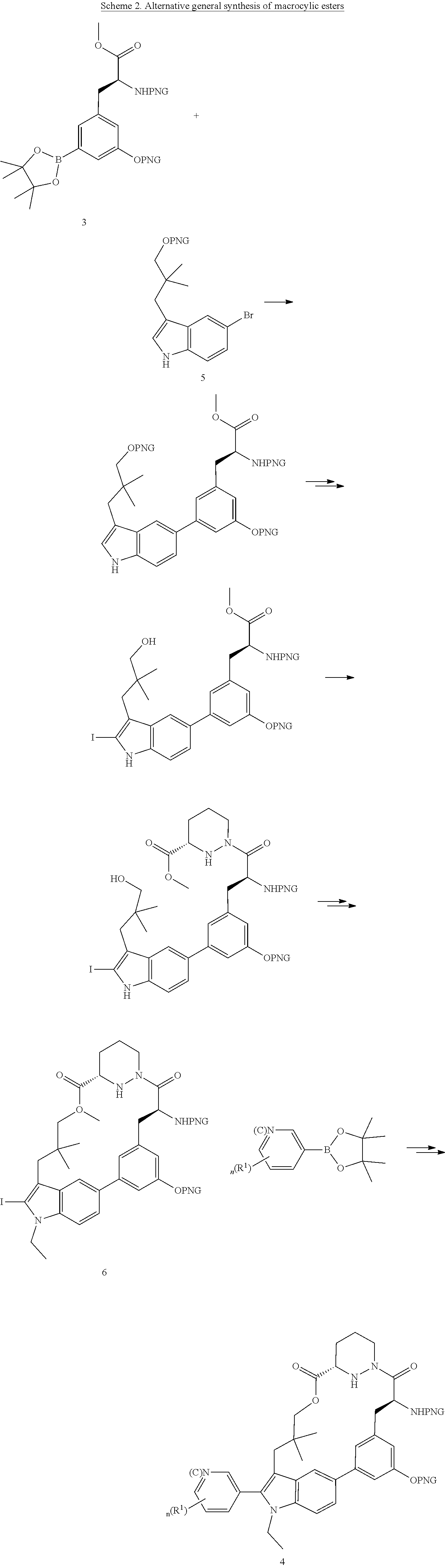

Alternatively, macrocyclic esters can be prepared as described in Scheme 2. An appropriately protected bromo-indolyl (5) can be coupled in the presence of Pd catalyst with boronic ester (3), followed by iodination, deprotection, and ester hydrolysis. Subsequent coupling with methyl(S)-hexahydropyridazine-3-carboxylate, followed by hydrolysis and macrolactonization can result in iodo intermediate (6). Coupling in the presence of Pd catalyst with an appropriately substituted boronic ester can yield fully a protected macrocycle (4). Additional deprotection or functionalization steps are required to produce a final compound. For example, a person of skill in the art would be able to install into a macrocyclic ester a desired-B-L-W group of a compound of Formula (I), where B, L and W are defined herein, including by using methods exemplified in certain Schemes below and in the Example section herein.

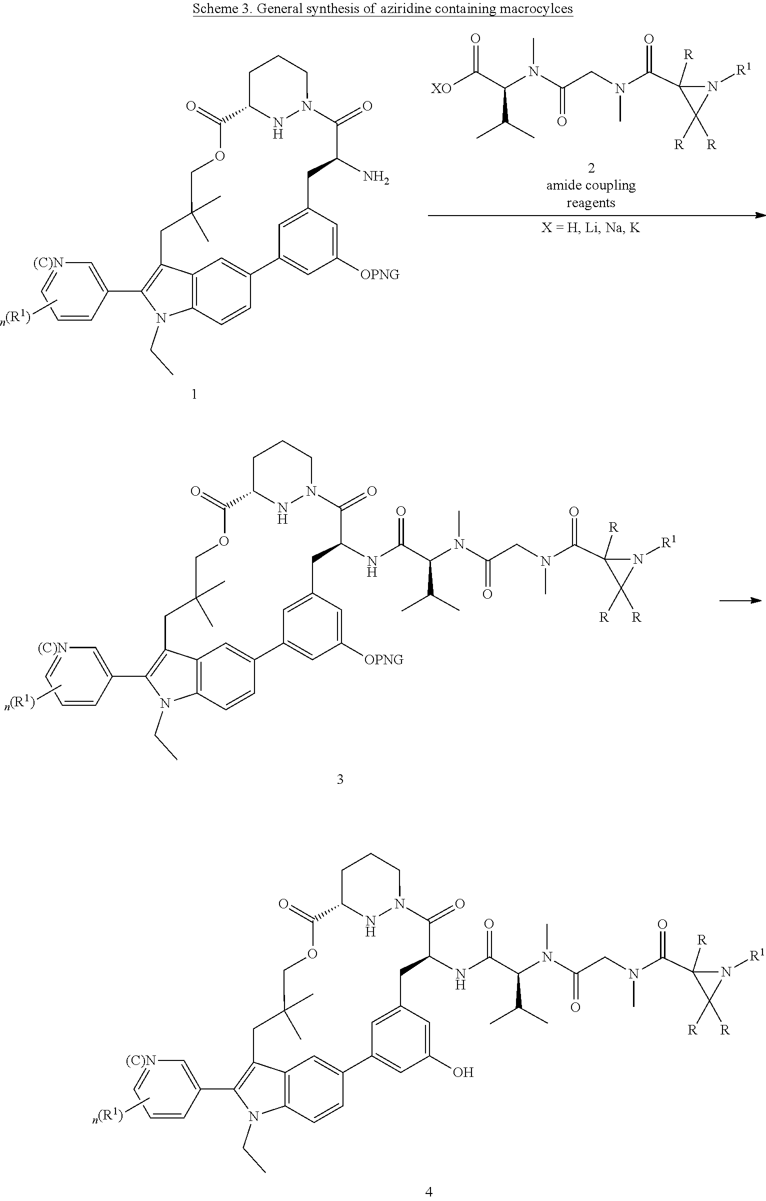

As shown in Scheme 3, compounds of this type may be prepared by the reaction of an appropriate amine (1) with an aziridine containing carboxylic acid (2) in the presence of standard amide coupling reagents, followed by deprotection of the aziridine, if R¹ is a protecting group, and deprotection of the phenol, if required, to produce the final compound (4).

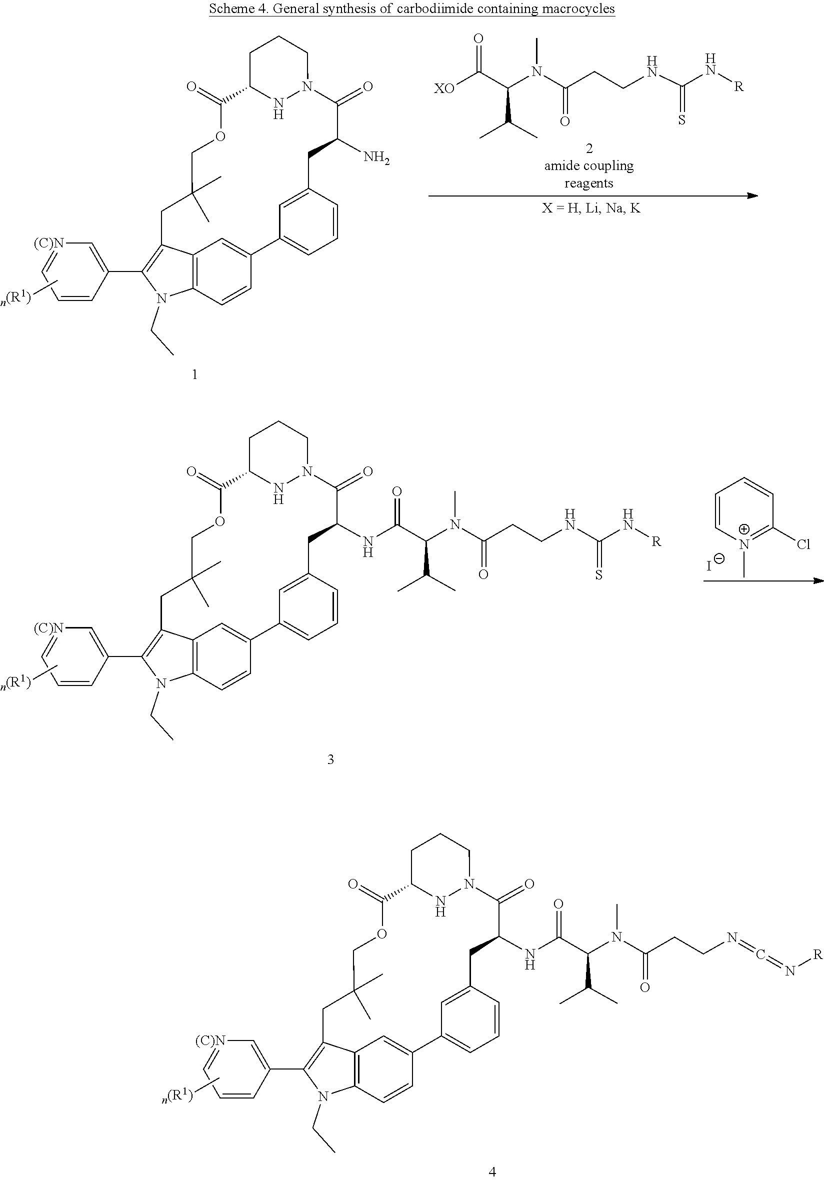

As shown in Scheme 4, compounds of this type may be prepared by the reaction of an appropriate amine (1) with a thiourea containing carboxylic acid (2) in the presence of standard amide coupling reagents, followed by conversion of the thiourea (3) to a carbodiimide (4) in the presence of 2-chloro-1-methylpyridin-1-ium iodide.

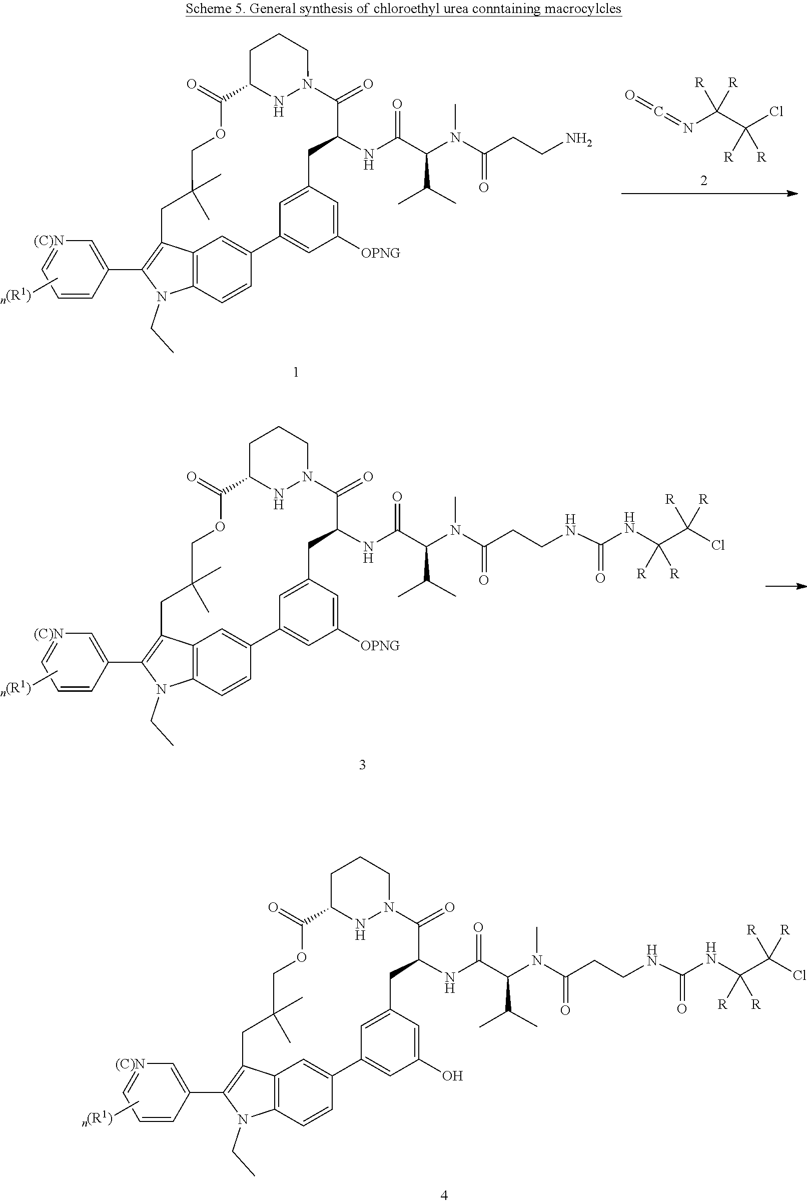

As shown in Scheme 5, compounds of this type may be prepared by the reaction of an appropriate amine (1) with an isocyanate (2) under basic conditions, followed by deprotection of the phenol, if required, to produce the final compound (4).

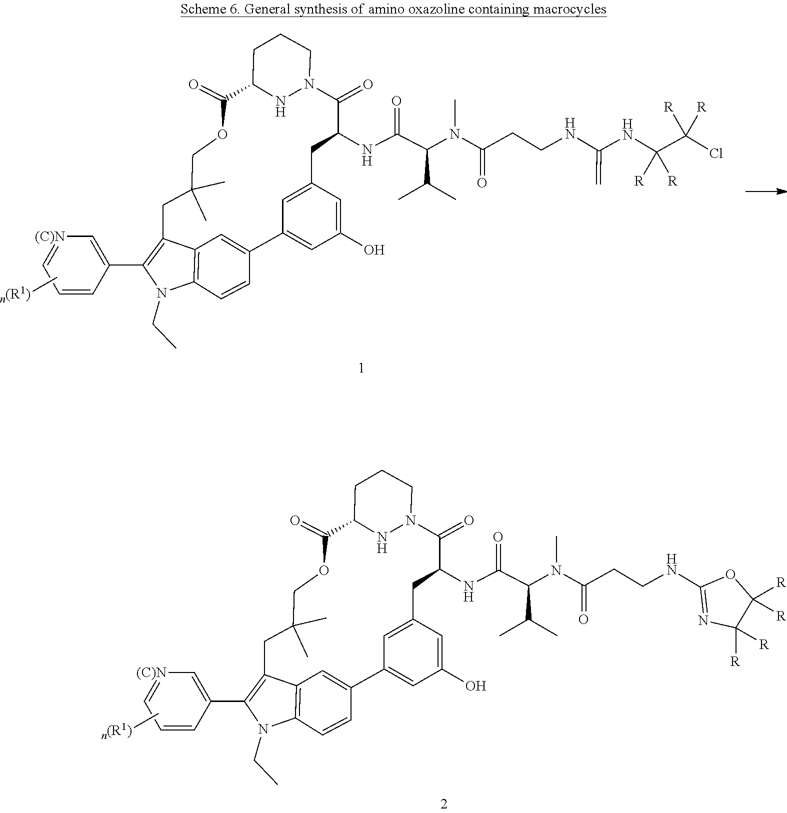

As shown in Scheme 6, compounds of this type may be prepared by cyclization of an appropriate chloroethyl urea (1) under elevated temperatures to produce the final compound (2).

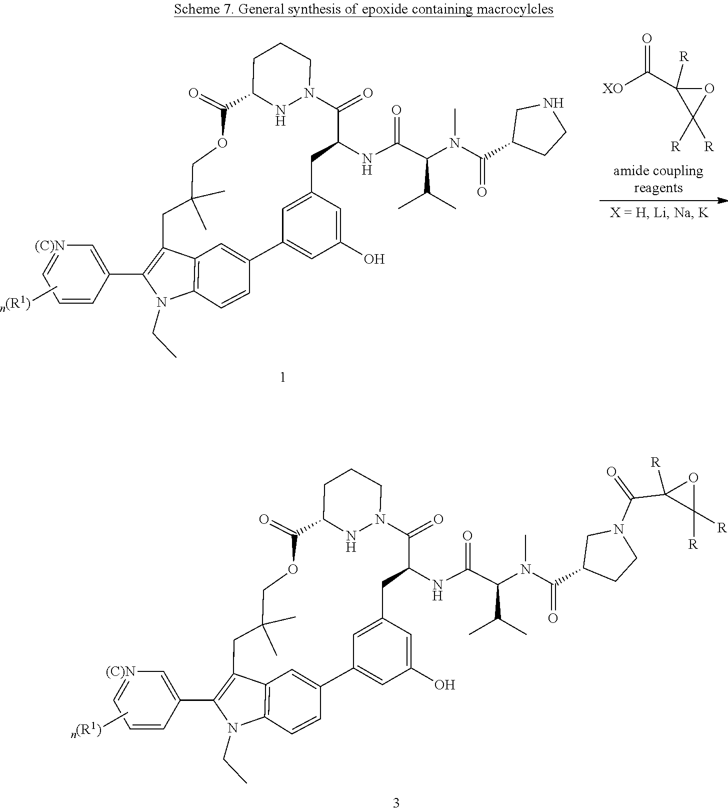

As shown in Scheme 7, compounds of this type may be prepared by the reaction of an appropriate amine (1) with an epoxide containing carboxylic acid (2) in the presence of standard amide coupling reagents to produce the final compound (3).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in certain Schemes above and in the Example section herein.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition, or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol, and the like. Such compositions may also contain amounts of nontoxic auxiliary substances such as wetting or emulsifying agents, pH buffering agents, and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery, and intranasal administration. Oral administration is also suitable for compounds of the invention, or pharmaceutically acceptable salts thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

### Numbered Embodiments

[1] A compound, or pharmaceutically acceptable salt thereof, having the structure of Formula I:

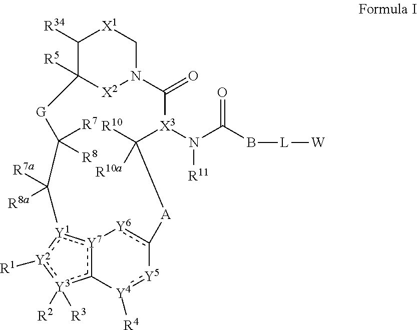

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N (R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O) NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂ or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl.
[2] The compound, or pharmaceutically acceptable salt thereof, of paragraph [1], wherein G is optionally substituted C₁-C₄ heteroalkylene.
[3] The compound, or pharmaceutically acceptable salt thereof, of paragraph [1] or [2], wherein the compound has the structure of Formula Ia:

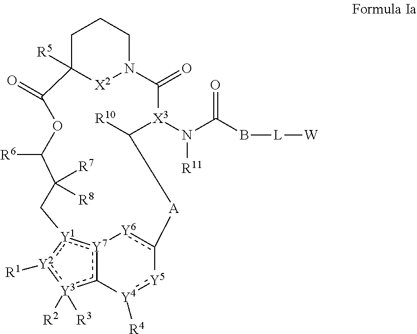

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; R¹⁰ is hydrogen, hydroxy, C₁-C₃ alkoxy, or C₁-C₃ alkyl; and R¹¹ is hydrogen or C₁-C₃ alkyl.
[4] The compound, or pharmaceutically acceptable salt thereof, of any one of paragraphs [1] to [3], wherein X² is NH.
[5] The compound, or pharmaceutically acceptable salt thereof, of any one of paragraphs [1] to [4], wherein X³ is CH.
[6] The compound, or pharmaceutically acceptable salt thereof, of any one of paragraphs [1] to [5], wherein R¹¹ is hydrogen.
[7] The compound, or pharmaceutically acceptable salt thereof, of any one of paragraphs [1] to [5], wherein R¹¹ is C₁-C₃ alkyl.
[8] The compound, or pharmaceutically acceptable salt thereof, of paragraph [7], wherein R¹¹ is methyl.
[9] The compound, or pharmaceutically acceptable salt thereof, of any one of paragraphs [1] to [6], wherein the compound has the structure of Formula Ib:

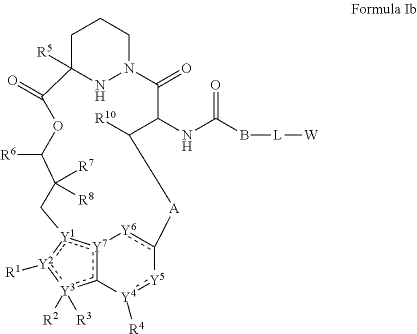

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
RT is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R&′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.
[10] The compound, or pharmaceutically acceptable salt thereof, of any one of paragraphs [1] to [9] wherein X¹ is optionally substituted C₁-C₂ alkylene.
[11] The compound, or pharmaceutically acceptable salt thereof, of paragraph [10], wherein X¹ is methylene.
[12] The compound, or pharmaceutically acceptable salt thereof, of any one of paragraphs [1] to [11], wherein R⁵ is hydrogen.
[13] The compound, or pharmaceutically acceptable salt thereof, of any one of paragraphs [1] to [11], wherein R⁵ is C₁-C₄ alkyl optionally substituted with halogen.
[14] The compound, or pharmaceutically acceptable salt thereof, of paragraph [13], wherein R⁵ is methyl.
[15] The compound, or pharmaceutically acceptable salt thereof, of any one of paragraphs [1] to [14], wherein Y⁴ is C.
[16] The compound, or pharmaceutically acceptable salt thereof, of any one of paragraphs [1] to [15], wherein R⁴ is hydrogen.
[17] The compound, or pharmaceutically acceptable salt thereof, of any one of paragraphs [1] to [16], wherein Y⁵ is CH.
[18] The compound, or pharmaceutically acceptable salt thereof, of any one of paragraphs [1] to [17], wherein Y⁶ is CH.
[19] The compound, or pharmaceutically acceptable salt thereof, of any one of paragraphs [1] to [18], wherein Y¹ is C.
[20] The compound, or pharmaceutically acceptable salt thereof, of any one of paragraphs [1] to [19], wherein Y² is C.
[21] The compound, or pharmaceutically acceptable salt thereof, of any one of paragraphs [1] to [20], wherein Y³ is N.
[22] The compound, or pharmaceutically acceptable salt thereof, of any one of paragraphs [1] to [21], wherein R³ is absent.
[23] The compound, or pharmaceutically acceptable salt thereof, of any one of paragraphs [1] to [22], wherein Y⁷ is C.
[24] The compound, or pharmaceutically acceptable salt thereof, of any one of paragraphs [1] to [6] or [9] to [23], wherein the compound has the structure of Formula Ic:

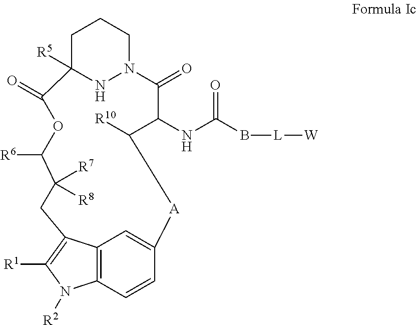

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.
[25] The compound, or pharmaceutically acceptable salt thereof, of any one of paragraphs [1] to [24], wherein R⁶ is hydrogen.
[26] The compound, or pharmaceutically acceptable salt thereof, of any one of paragraphs [1] to [25], wherein R² is hydrogen, cyano, optionally substituted C₁-C₆ alkyl, optionally substituted 3 to 6-membered cycloalkyl, or optionally substituted 3 to 6-membered heterocycloalkyl.
[27] The compound, or pharmaceutically acceptable salt thereof, of paragraph [26], wherein R² is optionally substituted C₁-C₆ alkyl.
[28] The compound, or pharmaceutically acceptable salt thereof, of paragraph [27], wherein R² is ethyl.
[29] The compound, or pharmaceutically acceptable salt thereof, of any one of paragraphs [1] to [28], wherein R⁷ is optionally substituted C₁-C₃ alkyl.
[30] The compound, or pharmaceutically acceptable salt thereof, of paragraph [29], wherein R⁷ is C₁-C₃ alkyl.
[31] The compound, or pharmaceutically acceptable salt thereof, of any one of paragraphs [1] to [30], wherein R⁸ is optionally substituted C₁-C₃ alkyl.
[32] The compound, or pharmaceutically acceptable salt thereof, of paragraph [31], wherein R⁸ is C₁-C₃ alkyl.
[33] The compound, or pharmaceutically acceptable salt thereof, of any one of paragraphs [1] to [32], wherein the compound has the structure of Formula Id:

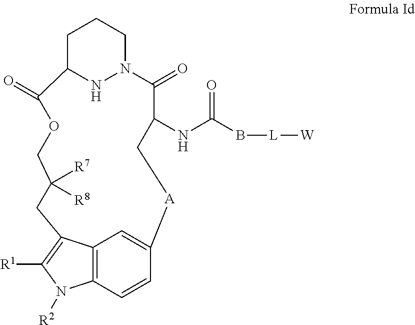

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.
[34] The compound, or pharmaceutically acceptable salt thereof, of any one of paragraphs [1] to [33], wherein R¹ is 5 to 10-membered heteroaryl.
[35] The compound, or pharmaceutically acceptable salt thereof, of paragraph [34], wherein R¹ is optionally substituted 6-membered aryl or optionally substituted 6-membered heteroaryl.
[36] The compound, or pharmaceutically acceptable salt thereof, of any one of paragraphs [1] to [35], wherein the compound has the structure of Formula I:

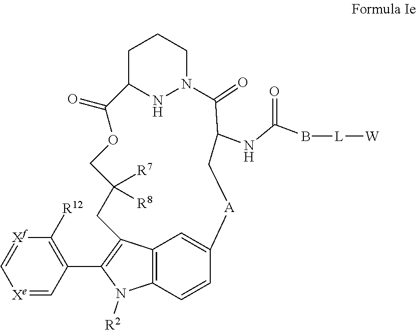

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} and X^{f} are, independently, N or CH; and
R¹² is optionally substituted C₁-C₆ alkyl or optionally substituted C₁-C₆ heteroalkyl, or optionally substituted 3 to 7-membered heterocycloalkyl.
[37] The compound, or pharmaceutically acceptable salt thereof, of paragraph [36], wherein X^{e} is N and X^{f} is CH.
[38] The compound, or pharmaceutically acceptable salt thereof, of paragraph [36], wherein X^{e} is CH and X^{f} is N.
[39] The compound, or pharmaceutically acceptable salt thereof, of any one of paragraphs [36] to [38], wherein R¹² is optionally substituted C₁-C₆ heteroalkyl.
[40] The compound, or pharmaceutically acceptable salt thereof, of any one of paragraphs [36] to [39], wherein R¹² is

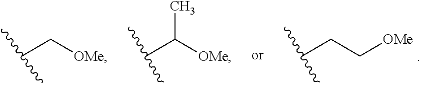

[41] The compound, or pharmaceutically acceptable salt thereof, of paragraph [1] or [2], wherein the compound has the structure of Formula VI:

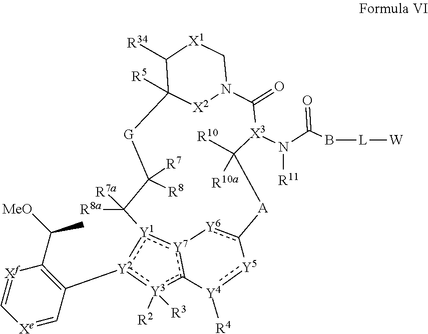

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N (R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O) NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R³⁴ is hydrogen or C₁-C₃ alkyl; and
X^{e} and X^{f} are, independently, N or CH.
[42] The compound, or pharmaceutically acceptable salt thereof, of paragraph [41], wherein the compound has the structure of Formula VIa:

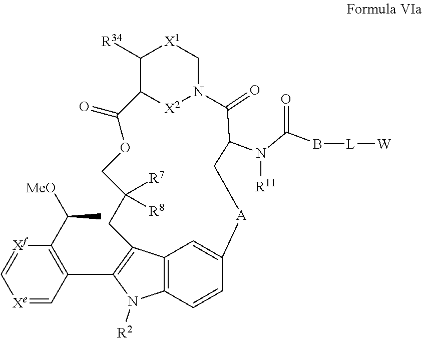

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.
[43] The compound, or pharmaceutically acceptable salt thereof, of paragraph [41] or [42], wherein the compound has the structure of Formula VIb:

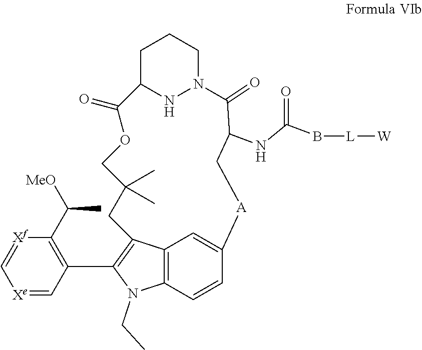

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.
[44] The compound, or pharmaceutically acceptable salt thereof, of any one of paragraphs [1] to [43], wherein A is optionally substituted 6-membered arylene.
[45] The compound, or pharmaceutically acceptable salt thereof, of paragraph [44], wherein A has the structure:

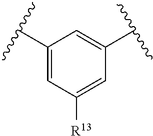

wherein R¹³ is hydrogen, hydroxy, amino, optionally substituted C₁-C₆ alkyl, or optionally substituted C₁-C₆ heteroalkyl.
[46] The compound, or pharmaceutically acceptable salt thereof, of paragraph [45], wherein R¹³ is hydrogen.
[47] The compound, or pharmaceutically acceptable salt thereof, of paragraph [45], wherein R¹³ is hydroxy.
[48] The compound, or pharmaceutically acceptable salt thereof, of any one of paragraphs [1] to [47], wherein B is —CHR⁹—
[49] The compound, or pharmaceutically acceptable salt thereof, of paragraph [48], wherein R⁹ is optionally substituted C₁-C₆ alkyl or optionally substituted 3 to 6-membered cycloalkyl.
[50] The compound, or pharmaceutically acceptable salt thereof, of paragraph [49], wherein R⁹ is:

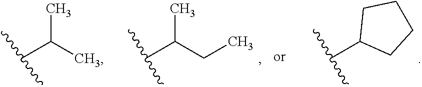

[51] The compound, or pharmaceutically acceptable salt thereof, of paragraph [50], wherein R⁹ is:

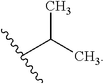 [SMILES: *C(C)C]

[52] The compound, or pharmaceutically acceptable salt thereof, of any one of paragraphs [1] to [47], wherein B is optionally substituted 6-membered arylene.
[53] The compound, or pharmaceutically acceptable salt thereof, of paragraph [52], wherein B is 6-membered arylene.
[54] The compound, or pharmaceutically acceptable salt thereof, of paragraph [53], wherein B is:

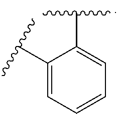 [SMILES: *c1ccccc1*]

[55] The compound, or pharmaceutically acceptable salt thereof, of any one of paragraphs [1] to [54], wherein R⁷ is methyl.
[56] The compound, or pharmaceutically acceptable salt thereof, of any one of paragraphs [1] to [55], wherein R⁸ is methyl.
[57] The compound, or pharmaceutically acceptable salt thereof, of any one of paragraphs [1] to [56], wherein the linker is the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between W and the linker; B¹, B², B³, and B⁴ each, independently, is selected from optionally substituted C₁-C₂ alkylene, optionally substituted C₁—Cs heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².
[58] The compound, or a pharmaceutically acceptable salt thereof, of any one of paragraphs [1] to [57], wherein the linker is acyclic.
[59] The compound, or a pharmaceutically acceptable salt thereof, of paragraph [58], wherein the linker has the structure of Formula IIa:

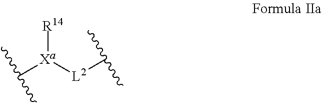

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene,
wherein at least one of X^{a}, R¹⁴, or L² is present.
[60] The compound, or a pharmaceutically acceptable salt thereof, of paragraph [59], wherein the linker has the structure:

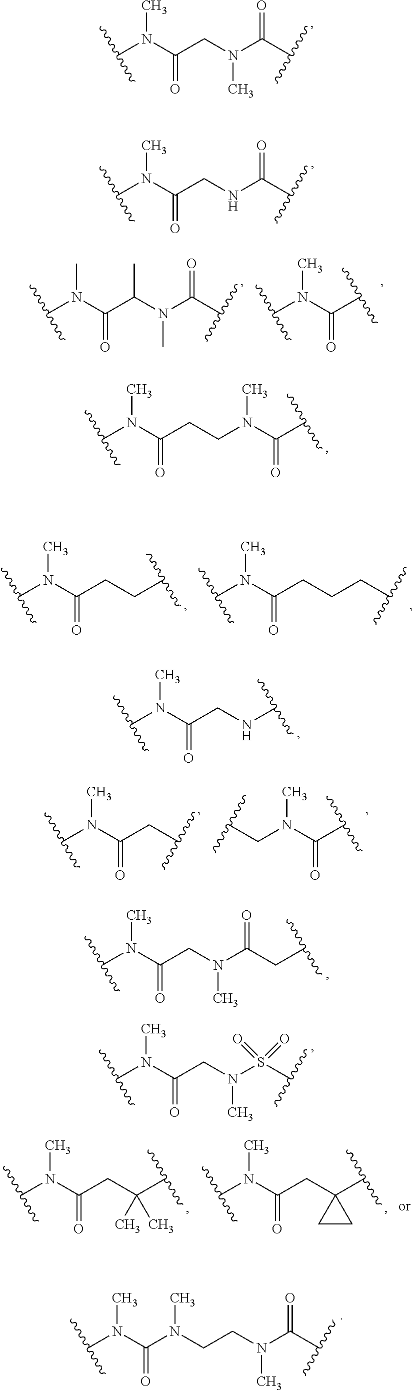

[61] The compound, or a pharmaceutically acceptable salt thereof, of any one of paragraphs [1] to [57], wherein the linker is or a comprises a cyclic group.
[62] The compound, or a pharmaceutically acceptable salt thereof, of any one of paragraphs [1] to [57] or [61], wherein the linker has the structure of Formula IIb:

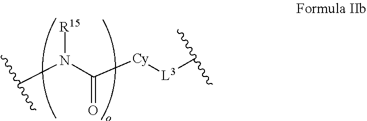

wherein o is 0 or 1;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.
[63] The compound, or a pharmaceutically acceptable salt thereof, of paragraph [62], wherein the linker has the structure:

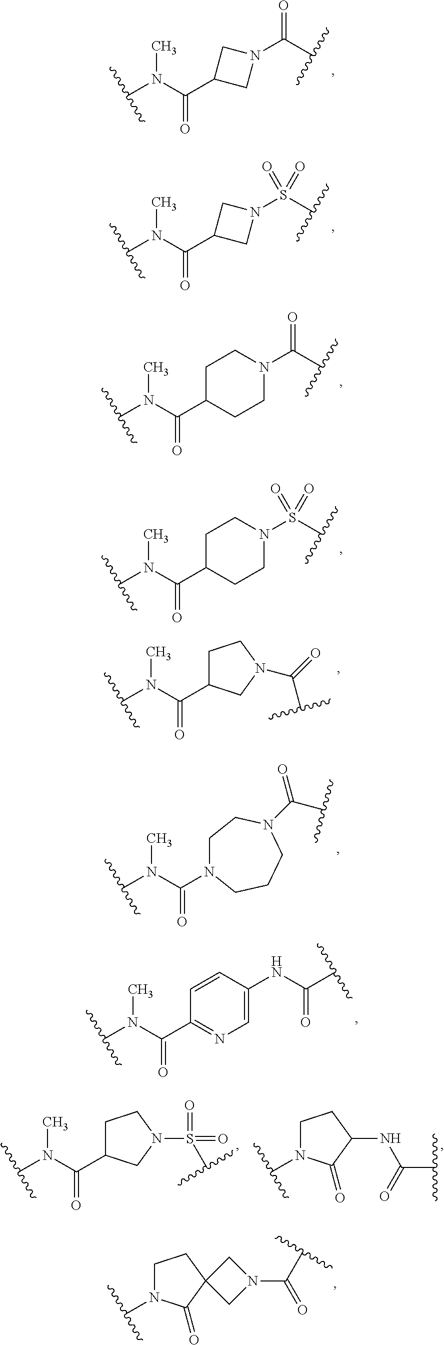

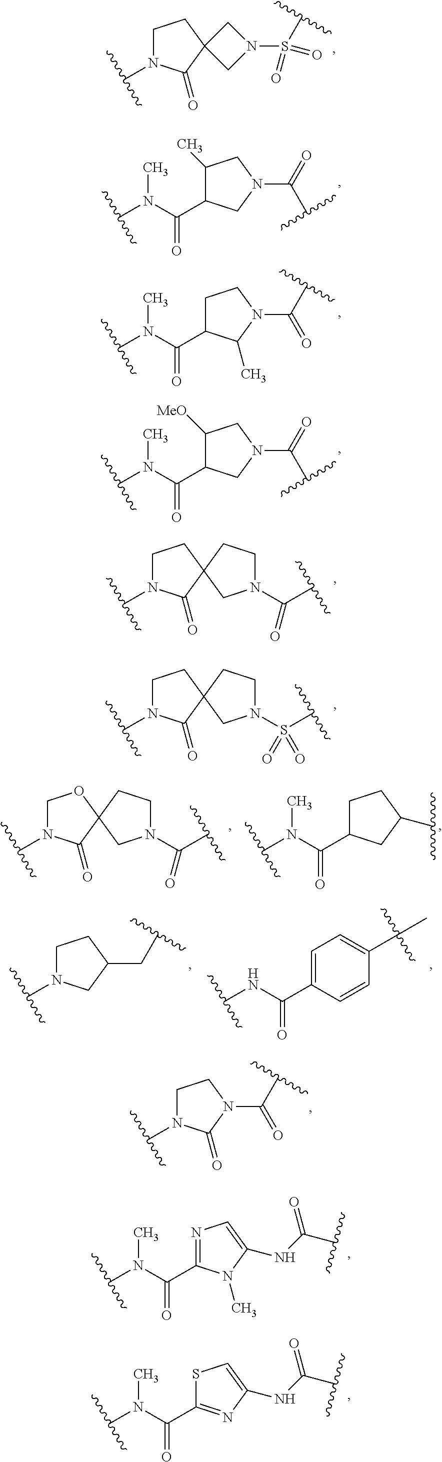

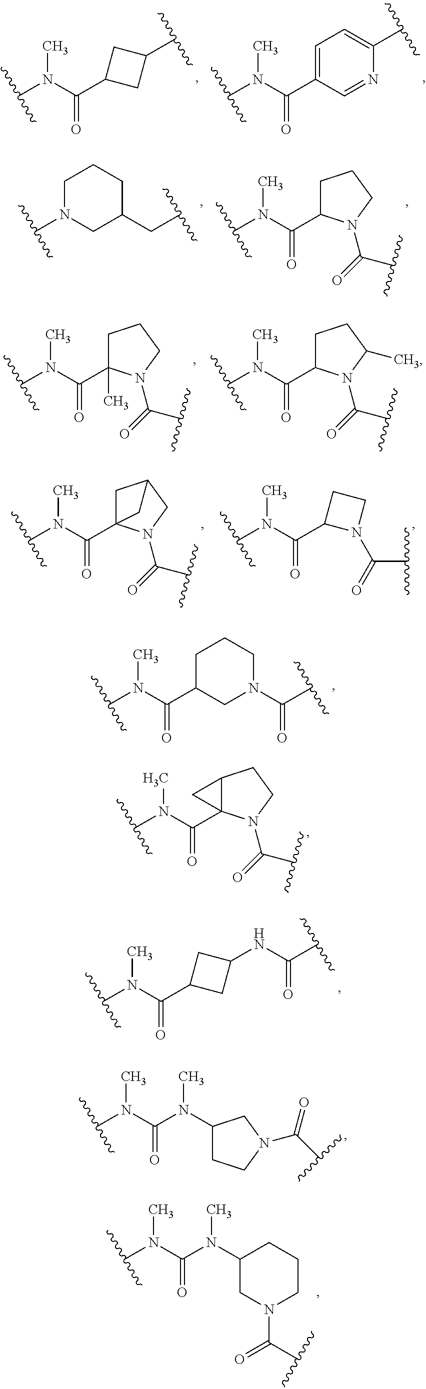

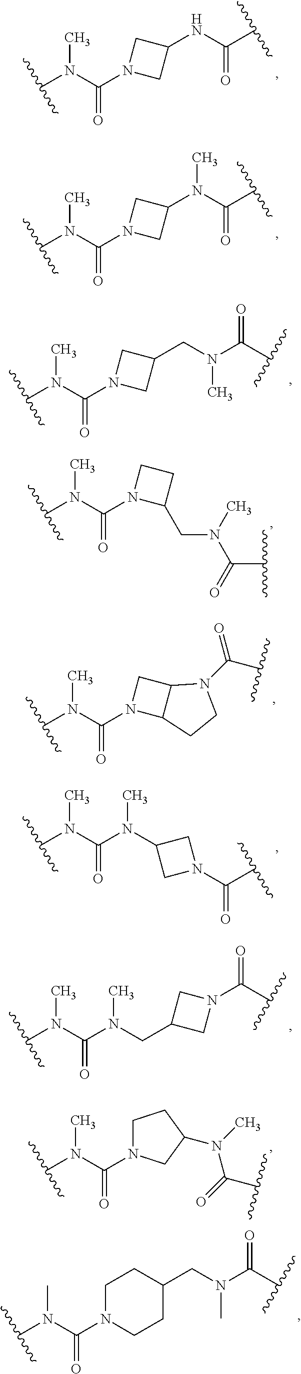

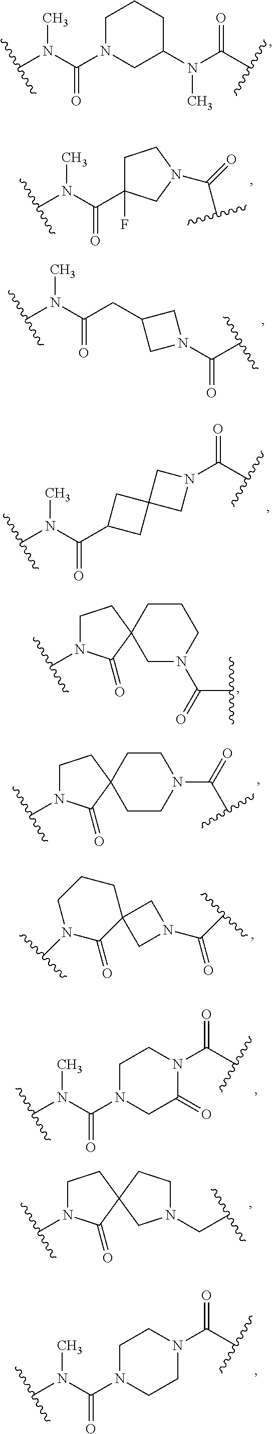

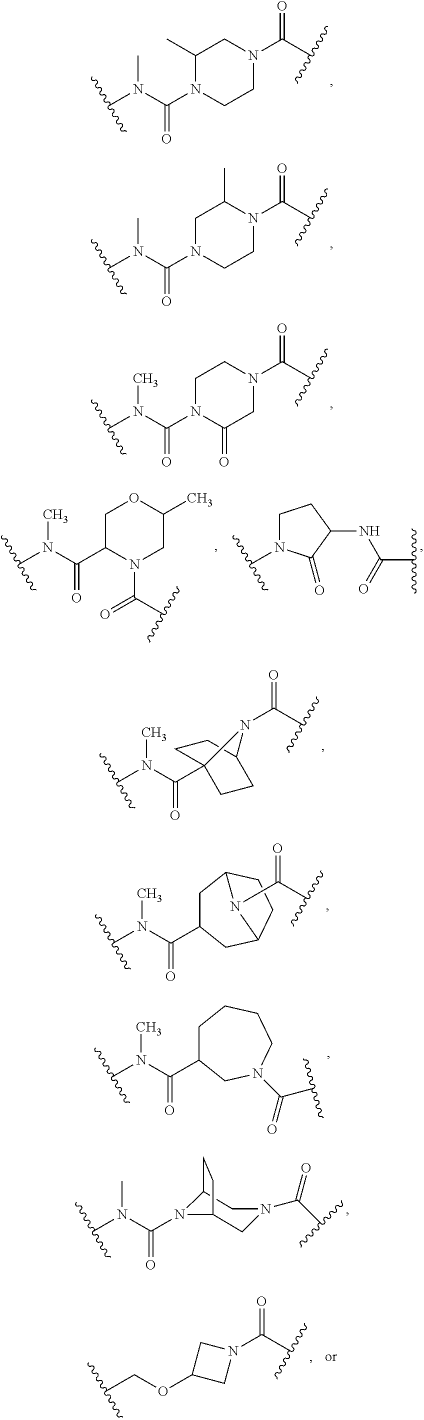

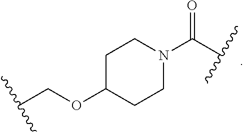 [SMILES: *COC1CCN(C(*)=O)CC1]

[64] The compound, or a pharmaceutically acceptable salt thereof, of any one of paragraphs [1] to [63], wherein W comprises a carbodiimide.
[65] The compound, or a pharmaceutically acceptable salt thereof, of paragraph [64], wherein W has the structure of Formula IIIa:

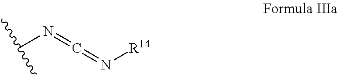

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl.
[66] The compound, or a pharmaceutically acceptable salt thereof, of paragraph [65], wherein W has the structure:

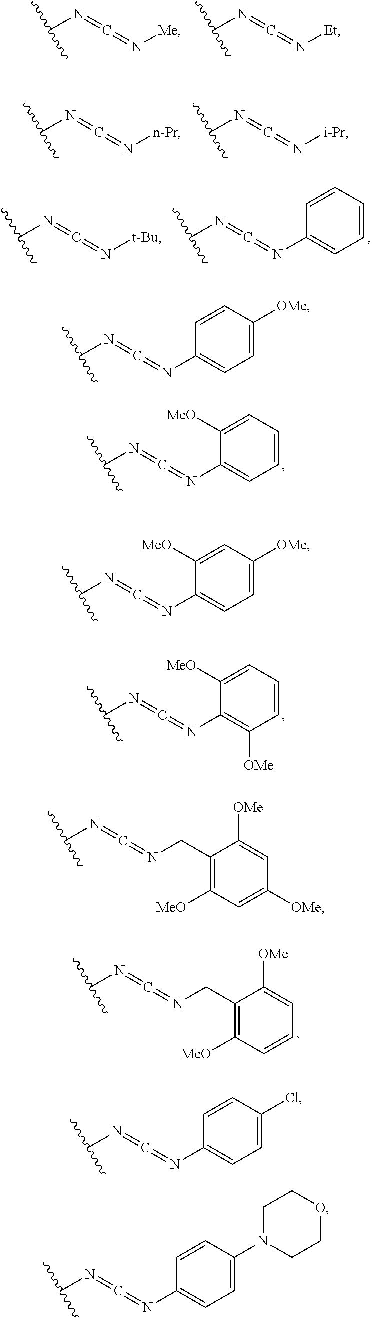

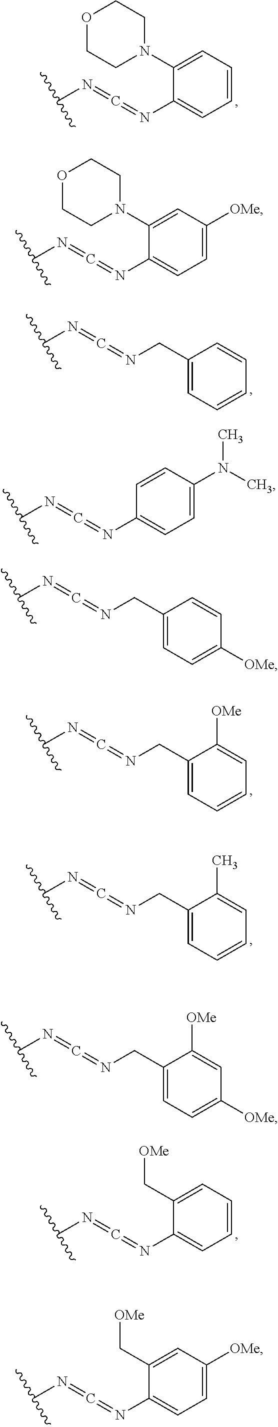

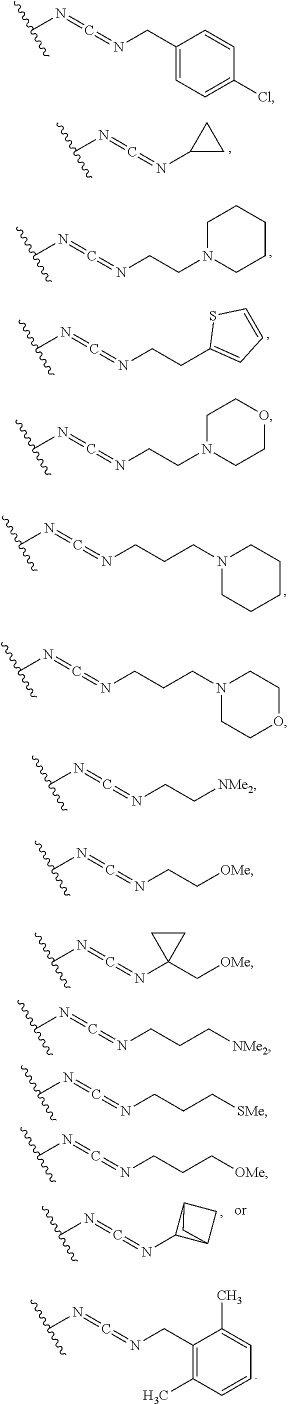

[67] The compound, or a pharmaceutically acceptable salt thereof, of any one of paragraphs [1] to [63], wherein W comprises an oxazoline or thiazoline.
[68] The compound, or a pharmaceutically acceptable salt thereof, of paragraph [67], wherein W has the structure of Formula IIIb:

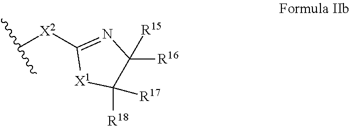

wherein X¹ is O or S;
X² is absent or NR¹⁹;
R¹⁵, R¹⁶, R¹⁷, and R¹⁸ are, independently, hydrogen or optionally substituted C₁-C₆ alkyl; and
R¹⁹ is hydrogen, C(O) (optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl.
[69] The compound, or a pharmaceutically acceptable salt thereof, of paragraph [68], wherein W is

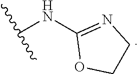 [SMILES: *NC1=NCCO1]

[70] The compound, or a pharmaceutically acceptable salt thereof, of any one of paragraphs [1] to [63], wherein W comprises a chloroethyl urea, a chloroethyl thiourea, a chloroethyl carbamate, or a chloroethyl thiocarbamate.
[71] The compound, or a pharmaceutically acceptable salt thereof, of paragraph [70], wherein W has the structure of Formula IIIc:

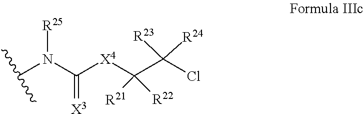

wherein X³ is O or S;
X⁴ is O, S, NR²⁶;
R²¹, R²², R²³, R²⁴, and R²⁶ are, independently, hydrogen or optionally substituted C₁-C₆ alkyl; and
R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl.
[72] The compound, or a pharmaceutically acceptable salt thereof, of paragraph [71], wherein W is

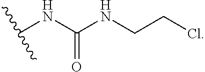 [SMILES: *NC(=O)NCCCl]

[73] The compound, or a pharmaceutically acceptable salt thereof, of any one of paragraphs [1] to [63], wherein W comprises an aziridine.
[74] The compound, or a pharmaceutically acceptable salt thereof, of paragraph [73], wherein W has the structure of Formula IIId1, Formula IIld2, Formula IIld3, or Formula IIld4:

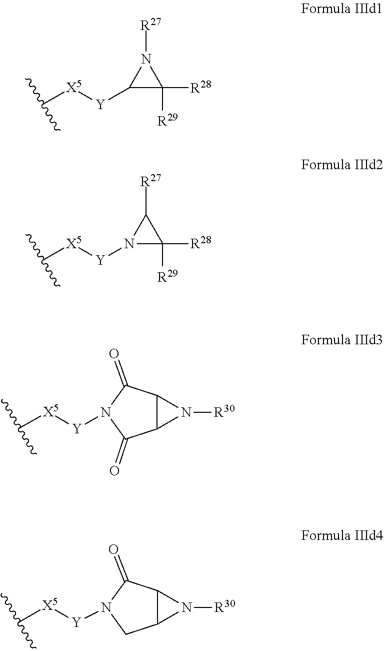

wherein X⁵ is absent or NR³⁰;
Y is absent or C(O), C(S), S (O), SO₂, or optionally substituted C₁-C₃ alkylene;
R²⁷ is hydrogen, —C(O)R³², —C(O)OR³², —SO₂R³³, —SOR³³, optionally substituted C₁-C₆ alkyl, optionally substituted 6 to 10-membered aryl, optionally substituted 3 to 14-membered heterocycloalkyl, or optionally substituted 5 to 10-membered heteroaryl;
R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;
each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;
R³⁰ is hydrogen or optionally substituted C₁-C₆ alkyl; and
R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl.
[75] The compound, or a pharmaceutically acceptable salt thereof, of paragraph [73] or [74], wherein W is:

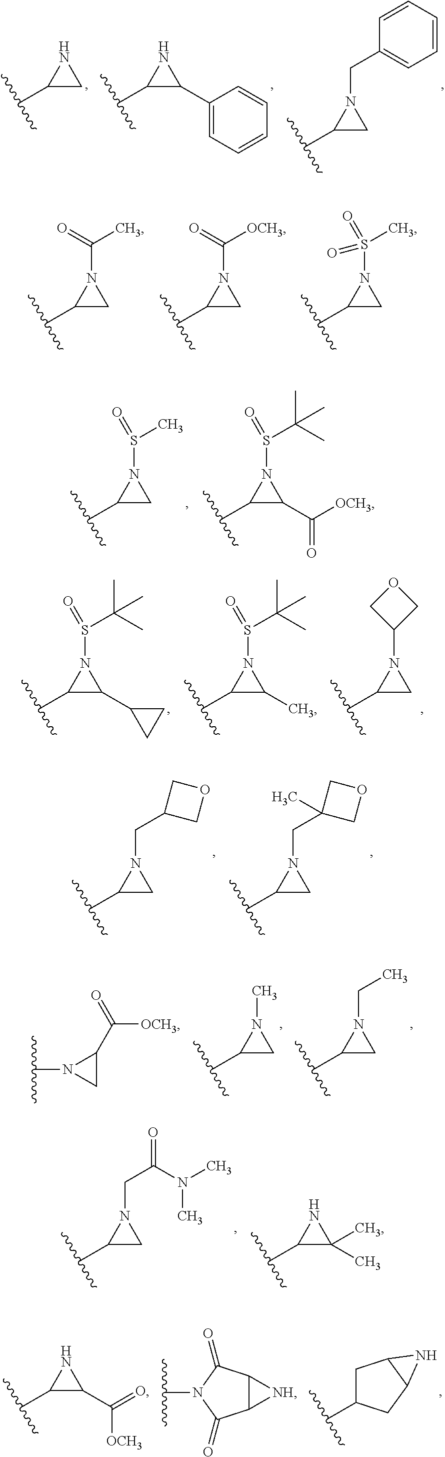

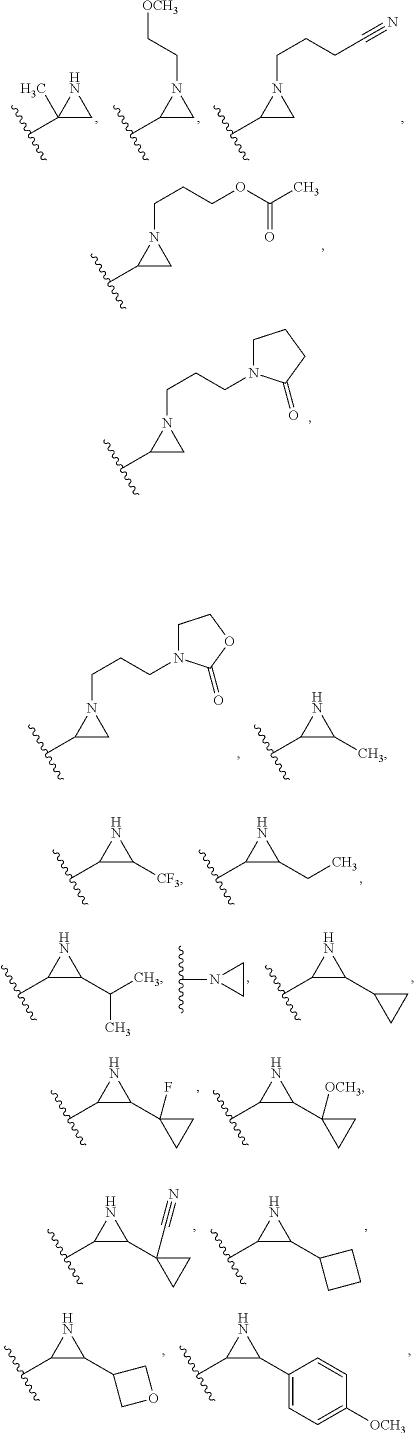

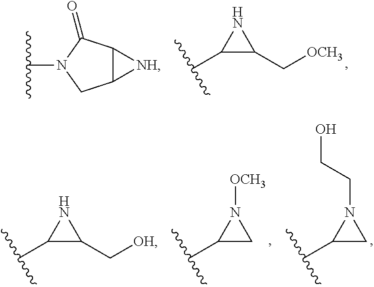 [SMILES: *N1CC2NC2C1=O ; *C1NC1COC ; *C1NC1CO ; *C1CN1OC ; *C1CN1CCO]

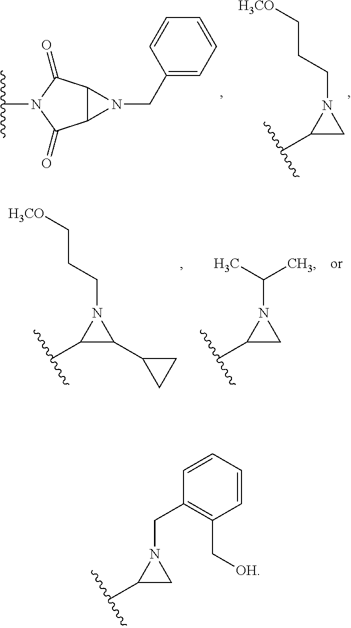

[76] The compound, or a pharmaceutically acceptable salt thereof, of any one of paragraphs [1] to [63], wherein W comprises an epoxide.
[77] The compound, or a pharmaceutically acceptable salt thereof, of paragraph [76], wherein W is

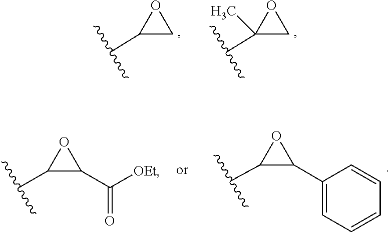

[78] A compound, or a pharmaceutically acceptable salt thereof, of Table 1 or Table 2.
[79] A pharmaceutical composition comprising a compound, or a pharmaceutically acceptable salt thereof, of any one of paragraphs [1] to [78] and a pharmaceutically acceptable excipient.
[80] A conjugate, or salt thereof, comprising the structure of Formula IV:

M-L-P Formula IV

wherein L is a linker;
P is a monovalent organic moiety; and
M has the structure of Formula V:

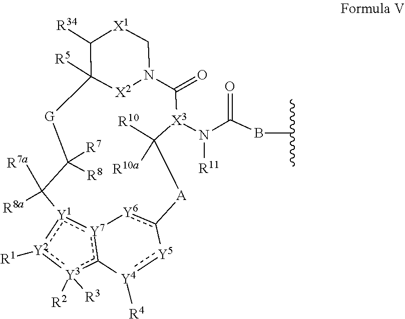

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of —N (R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷R⁸′; C═N (OH), C═N (O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R^{10a} is hydrogen or halo;
R¹¹ is hydrogen or C₁-C₃ alkyl; and
R³⁴ is hydrogen or C₁-C₃ alkyl.
[81] A conjugate, or salt thereof, of paragraph [80], wherein M has the structure of Formula Vc:

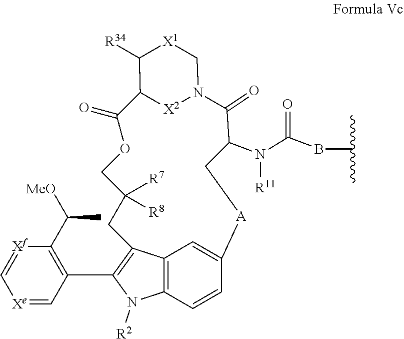

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
X^{e} and X^{f} are, independently, N or CH;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹¹ is hydrogen or C₁-C₃ alkyl; and
R³⁴ is hydrogen or C₁-C₃ alkyl.

In some embodiments of a compound of the present invention, X^{e} is N and X^{f} is CH. In some embodiments, X^{e} is CH and X^{f} is N.
[82] The conjugate, or salt thereof, of paragraph [80] or [81], wherein M has the structure of Formula Vd:

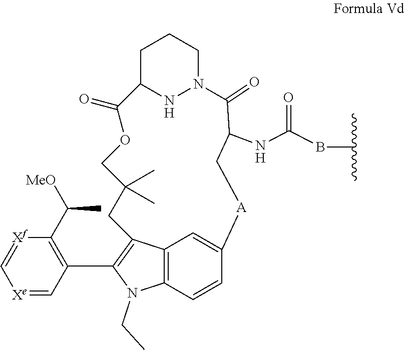

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.
[83] The conjugate, or salt thereof, of any one of paragraphs [80] to [82], wherein the linker has the structure of Formula II:

A¹-(B¹)_{f}—(C¹)_{g}—(B²)ₕ-(D¹)-(B³)ᵢ—(C²)ⱼ—(B⁴)ₖ-A² Formula II
where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².
[84] The conjugate, or salt thereof, of any one of paragraphs [80] to [83], wherein the monovalent organic moiety is a protein.
[85] The conjugate, or salt thereof, of paragraph [84], wherein the protein is a Ras protein.
[86] The conjugate, or salt thereof, of paragraph [85], wherein the Ras protein is K-Ras G12D or K-Ras G13D.
[87] The conjugate, or salt thereof, of any one of paragraphs [80] to [86], wherein the linker is bound to the monovalent organic moiety through a bond to a carboxyl group of an amino acid residue of the monovalent organic moiety.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:

| | |
|---|---|
| CH₂Cl₂, DCM | Methylene chloride, Dichloromethane |
| CH₃CN, MeCN | Acetonitrile |
| CuI | Copper (I) iodide |
| DIPEA | Diisopropylethyl amine |
| DMF | N,N-Dimethylformamide |
| EtOAc | Ethyl acetate |
| h | hour |
| H₂O | Water |
| HCl | Hydrochloric acid |
| K₃PO₄ | Potassium phosphate (tribasic) |
| MeOH | Methanol |
| Na₂SO₄ | Sodium sulfate |
| NMP | N-methyl pyrrolidone |
| Pd(dppf)Cl₂ | [1,1′-Bis(diphenylphos- |
| | phino)ferrocene]dichloropalladium(II) |

### Instrumentation

Mass spectrometry data collection took place with a Shimadzu LCMS-2020 or Waters Acquity UPLC with either a QDa detector or SQ Detector 2. Samples were injected in their liquid phase onto a C-18 reverse phase column to remove assay buffer and prepare the samples for the mass spectrometer. The compounds were eluted from the column using an acetonitrile gradient and fed into the mass analyzer. Initial data analysis took place with either Shimadzu LabSolutions or Waters MassLynx. NMR data was collected with either a Bruker AVANCE III HD 400 MHz or a Bruker Ascend 500 MHz instrument and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

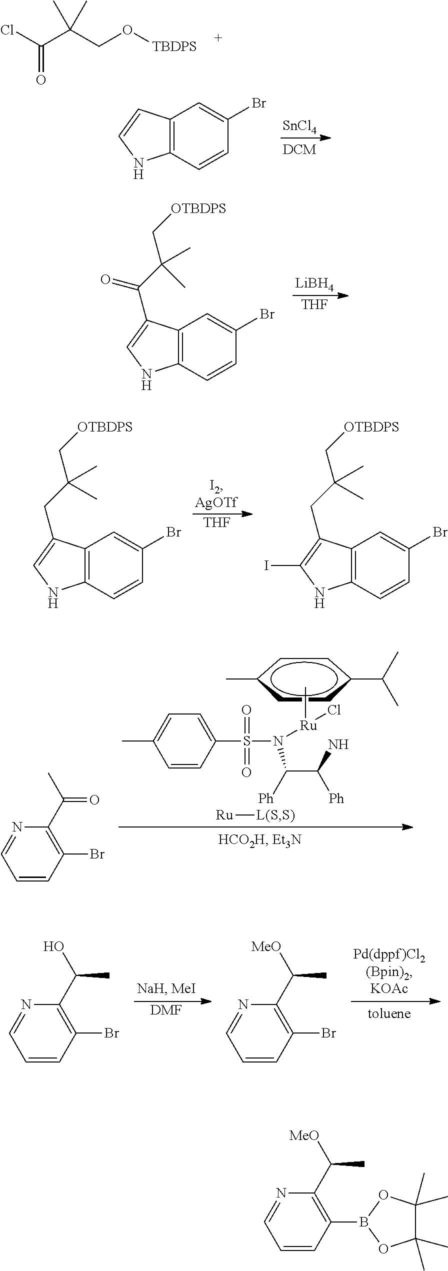

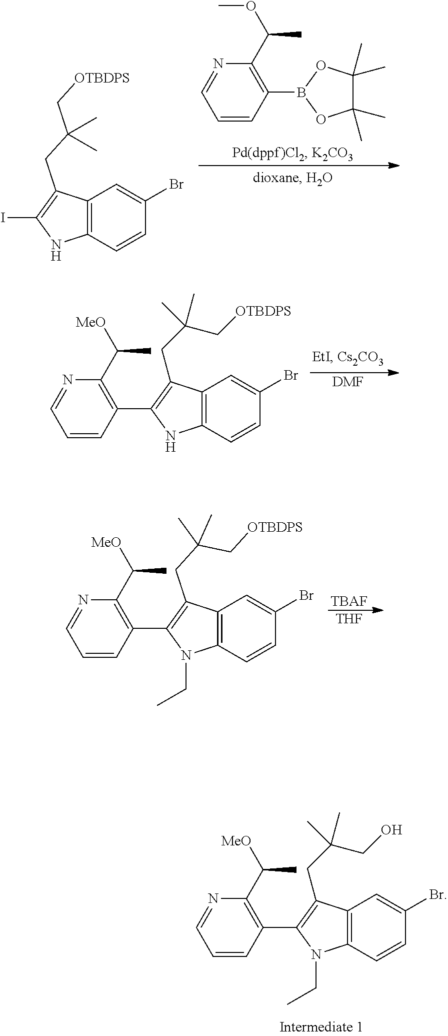

### Step 1: Synthesis of 1-(5-bromo-1H-indol-3-yl)-3-((tert-butyldiphenylsilyl)oxy)-2,2-dimethylpropan-1-one

To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (4×100 mL), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI) m/z: [M+Na] calcd for C₂₉H₃₂BrNO₂SiNa 556.1; found 556.3.

### Step 2: Synthesis of 1-(5-bromo-1H-indol-3-yl)-3-((tert-butyldiphenylsilyl)oxy)-2,2-dimethylpropan-1-one

To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH·H₂O (890 mg, 4.7 mmol) were added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI) m/z: [M+H] calcd for C₂₉H₃₄BrNOSi: 519.2; found 520.1; ¹H NMR (400 MHZ, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

### Step 3: Synthesis of 5-bromo-3-(3-((tert-butyldiphenylsilyl)oxy)-2,2-dimethylpropyl)-2-iodo-1H-indole

To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at room temperature was added AgOTf (888 mg, 3.5 mmol). The mixture was stirred at room temperature for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. 1H NMR (400 MHZ, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

### Step 4: Synthesis of (1S)-1-(3-bromopyridin-2-yl) ethanol

To a stirred mixture of HCOOH (66.3 g, 1.44 mol) in Et₃N (1002 mL, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to room temperature and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl) ethanol (100 g, 74% yield) a an oil. LCMS (ESI) m/z: [M+H] calcd for C₇H₈BrNO: 201.98; found 201.9.

### Step 5: Synthesis of 3-bromo-2-[(1S)-1-methoxyethyl]pyridine

To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to room temperature and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI) m/z: [M+H] calcd for C&H₁₀BrNO: 215.99; found 215.9.

### Step 6: Synthesis of 2-[(1S)-1-methoxyethyl]-3-(4,4,5,5-tetramethyl-1,3,2-dioxaborolan-2-yl) pyridine

To a stirred mixture of 3-bromo-2-[(1 S)-1-methoxyethyl]pyridine (90 g, 417 mmol) in toluene (900 mL) at room temperature under an atmosphere of Ar was added bis (pinacolato)diboron (127 g, 500 mmol) and KOAc (81.8 g, 833 mmol) and Pd(dppf)Cl₂ (30.5 g, 41.7 mmol). The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl) pyridine (100 g, 63% yield) as a semi-solid. LCMS (ESI) m/z: [M+H] calcd for C₁₄H₂₂BNO₃: 264.17; found 264.1.

### Step 7: Synthesis of 5-bromo-3-[3-[(tert-butyldiphenylsilyl)oxy]-2,2-dimethylpropyl]-2-[2-[(1S)-1-methoxyethyl]pyridin-3-yl]-1H-indole

To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl) pyridine (100 g, 380 mmol) in 1,4-dioxane (1.4 L) at room temperature under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1 S)-1-methoxyethyl]pyridin-3-yl]-1H-indole (71 g, 45% yield) as a solid. LCMS (ESI) m/z: [M+H] calcd for C₃₇H₄₃BrN₂O₂Si: 655.23; found 655.1.

### Step 8: Synthesis of 5-bromo-3-[3-[(tert-butyldiphenylsilyl)oxy]-2,2-dimethylpropyl]-1-ethyl-2-[2-[(1S)-1-methoxyethyl]pyridin-3-yl]indole

To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to room temperature and stirred for 16 h then H₂O (4 L) added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1 S)-1-methoxyethyl]pyridin-3-yl]indole (66 g, 80% yield) as an oil. LCMS (ESI) m/z: [M+H] calcd for C₃₉H₄₇BrN₂O₂Si: 683.26; found 683.3.

### Step 9: Synthesis of 3-(5-bromo-1-ethyl-2-[2-[(1S)-1-methoxyethyl]pyridin-3-yl]indol-3-yl)-2,2-dimethylpropan-1-ol

To a stirred mixture of TBAF (172.6 g, 660 mmol) in THF (660 mL) at room temperature under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI) m/z: [M+H] calcd for C₂₃H₂₉BrN₂O₂: 445.14; found 445.1.

### Intermediate 1. Alternative Synthesis through Fisher Indole Route

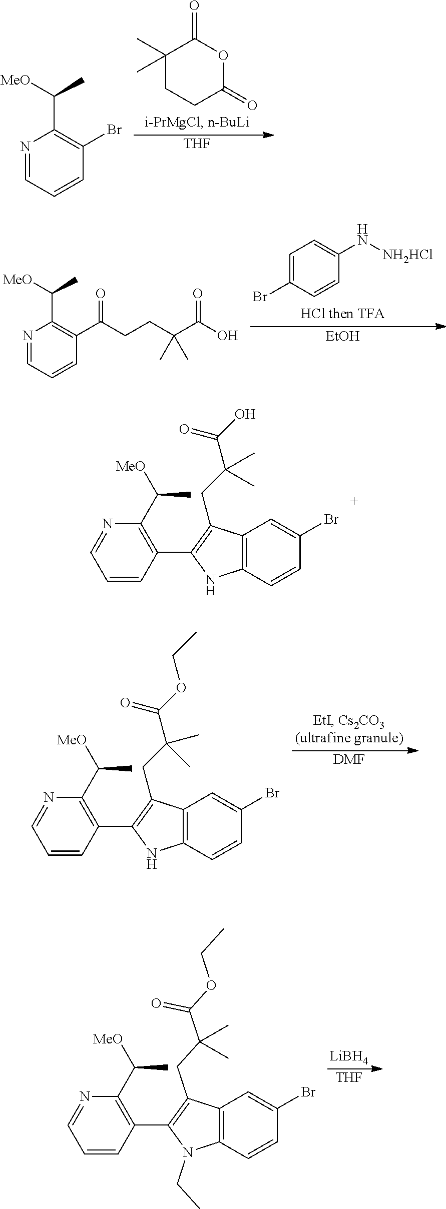

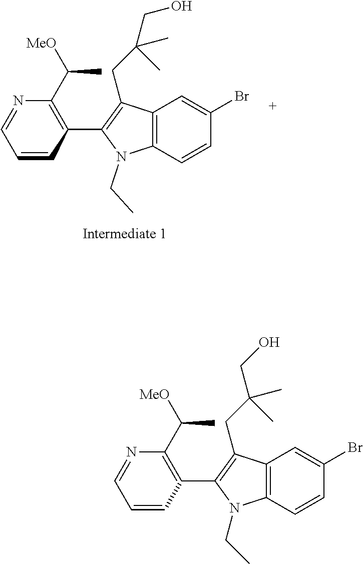

### Step 1: Synthesis of 5-[2-[(1 S)-1-methoxyethyl]pyridin-3-yl]-2,2-dimethyl-5-oxopentanoic acid

To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with H₂O (3 L) at 0° C. and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1 S)-1-methoxyethyl]pyridin-3-yl]-2,2-dimethyl-5-oxopentanoic acid (87 g, 34% yield) as a solid. LCMS (ESI) m/z: [M+H] calcd for C₁₅H₂₁NO₄: 280.15; found 280.1.

### Step 2: Synthesis of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate

To a mixture of 5-[2-[(1S)-1-methoxyethyl]pyridin-3-yl]-2,2-dimethyl-5-oxopentanoic acid (78 g, 279 mmol) in EtOH (0.78 L) at room temperature under an atmosphere of N₂ was added (4-bromophenyl) hydrazine HCl salt (68.7 g, 307 mmol) in portions. The mixture was heated to 85° C. and stirred for 2 h, cooled to room temperature, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5 h, concentrated under reduced pressure and the residue adjusted to PH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (78 g, crude). LCMS (ESI) m/z: [M+H] calcd for C₂₁H₂₃BrN₂O₃: 430.1 and C₂₃H₂₇BrN₂O₃: 459.12; found 431.1 (carboxylic acid) and 459.1.

### Step 3: Synthesis of ethyl 3-(5-bromo-1-ethyl-2-[2-[(1 S)-1-methoxyethyl]pyridin-3-yl]indol-3-yl)-2,2-dimethylpropanoate

To a mixture of 3-(5-bromo-2-[2-[(1 S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (198 g, 459 mmol) in DMF (1.8 L) at 0° C. under an atmosphere of N₂ was added CS₂CO₃ (449 g, 1.38 mol) in portions. EtI (215 g, 1.38 mmol) in DMF (200 mL) was then added dropwise at 0° C. The mixture was warmed to room temperature and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI) m/z: [M+H] calcd for C₂₅H₃₁BrN₂O₃: 487.17; found 487.2.

### Step 4: Synthesis of 3-(5-bromo-1-ethyl-2-(2-((S)-1-methoxyethyl) pyridin-3-yl)-1H-indol-3-yl)-2,2-dimethylpropan-1-ol

To a mixture of ethyl 3-(5-bromo-1-ethyl-2-[2-[(1 S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl) pyridin-3-yl)-1H-indol-3-yl)-2,2-dimethylpropan-1-ol (as single atropisomers) (60 g, 38% yield) and (40 g, 26% yield) both as solids. LCMS (ESI) m/z: [M+H] calcd for C₂₃H₂₉BrN₂O₂: 445.14; found 445.2.

### Intermediate 2 and Intermediate 4. Synthesis of(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate

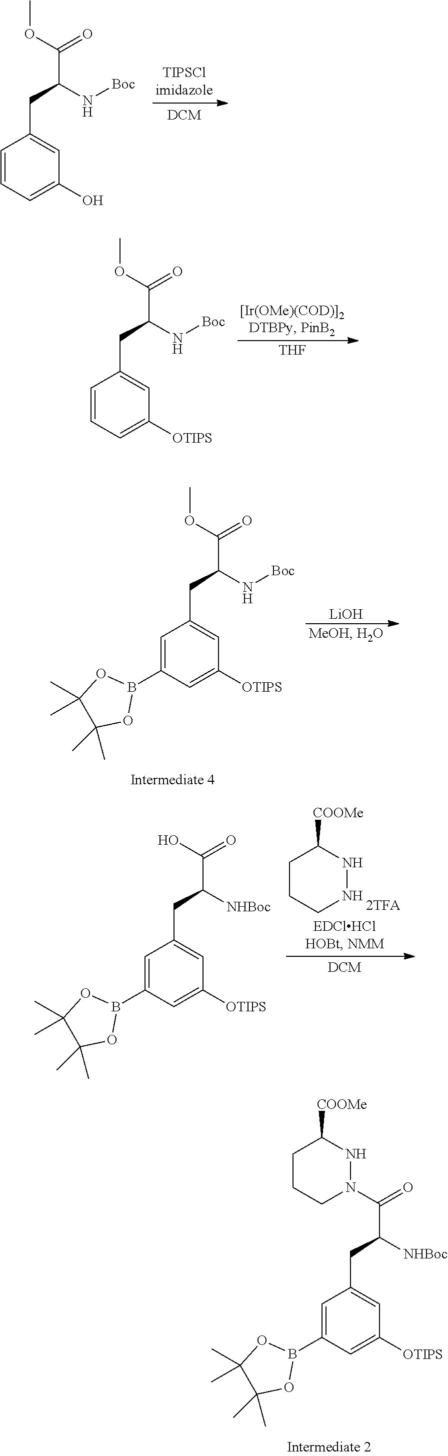

### Step 1: Synthesis of(S)-methyl 2-(tert-butoxycarbonylamino)-3-(3-(triisopropylsilyloxy)phenyl)-propanoate

To a mixture of(S)-methyl 2-(tert-butoxycarbonylamino)-3-(3-hydroxyphenyl) propanoate (10.0 g, 33.9 mmol) in DCM (100 mL) was added imidazole (4.6 g, 67.8 mmol) and TIPSCl (7.8 g, 40.7 mmol). The mixture was stirred at room temperature overnight then diluted with DCM (200 mL) and washed with H₂O (3×150 mL). The organic layer was dried over anhydrous Na₂SO₄, filtered, concentrated under reduced pressure and the residue was purified by silica gel column chromatography to give (S)-methyl 2-(tert-butoxycarbonylamino)-3-(3-(triisopropylsilyloxy)phenyl)-propanoate (15.0 g, 98% yield) as an oil. LCMS (ESI) m/z: [M+Na] calcd for C₂₄H₄₁NO₅SiNa: 474.22; found 474.2.

### Step 2: Synthesis of(S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate

A mixture of(S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]₂ (1.1 g, 1.7 mmol) and 4-tert-butyl-2-(4-tert-butyl-2-pyridyl) pyridine (1.3 g, 5.0 mmol) was purged with Ar, then THF (75 mL) was added and the mixture placed under an atmosphere of Ar and sealed. The mixture was heated to 80° C. and stirred for 16 h, concentrated under reduced pressure, and the residue was purified by silica gel column chromatography to give (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate (7.5 g, 78% yield) as a solid. LCMS (ESI) m/z: [M+Na] calcd for C₃₀H₅₂BNO₇SiNa: 600.35; found 600.4; ¹H NMR (300 MHz, CD₃OD) δ 7.18 (s, 1H), 7.11 (s, 1H), 6.85 (s, 1H), 4.34 (m, 1H), 3.68 (s, 3H), 3.08 (m, 1H), 2.86 (m, 1H), 1.41-1.20 (m, 26H), 1.20-1.01 (m, 22H), 0.98-0.79 (m, 4H).

### Step 3: Synthesis of(S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid

To a mixture of triisopropylsilyl(S)-2-((tert-butoxycarbonyl) amino)-3-(3-(4,4,5,5-tetramethyl-1,3,2-dioxaborolan-2-yl)-5-((triisopropylsilyl)oxy) phenyl) propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to PH ˜5 with 1M HCl and extracted with EtOAc (2×250 mL). The combined organic layers were washed with brine (3×100 mL), dried over anhydrous Na₂SO₄, filtered and the filtrate concentrated under reduced pressure to give (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (3.7 g, 95% yield), which was used directly in the next step without further purification. LCMS (ESI) m/z: [M+NH₄] calcd for C₂₉H₅₀BNOSiNH₄: 581.38; found 581.4.

### Step 4: Synthesis of methyl(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate

To a mixture of methyl(S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl) amino)-3-(3-(4,4,5,5-tetramethyl-1,3,2-dioxaborolan-2-yl)-5-((triisopropylsilyl)oxy) phenyl) propanoic acid (24 g, 42.6 mmol) in DCM (50 mL) then HOBt (1.21 g, 9.0 mmol) and EDCI HCl salt (12.9 g, 67.6 mmol). The mixture was warmed to room temperature and stirred for 16 h, then diluted with DCM (200 mL) and washed with H₂O (3×150 mL). The organic layer was dried over anhydrous Na₂SO₄, filtered, the filtrate concentrated under reduced pressure and the residue was purified by silica gel column chromatography to give methyl(S)-1-((S)-2-((tert-butoxycarbonyl) amino)-3-(3-(4,4,5,5-tetramethyl-1,3,2-dioxaborolan-2-yl)-5-((triisopropylsilyl)oxy) phenyl) propanoyl) hexahydropyridazine-3-carboxylate (22 g, 71% yield) as an oil. LCMS (ESI) m/z: [M+H] calcd for C₃₅H₆₀BN₃O₈Si: 690.42; found 690.5.

### Intermediate 3. Synthesis of(S)-tert-butyl 3-methyl-2-((S)—N-methylpyrrolidine-3-carboxamido) butanoate

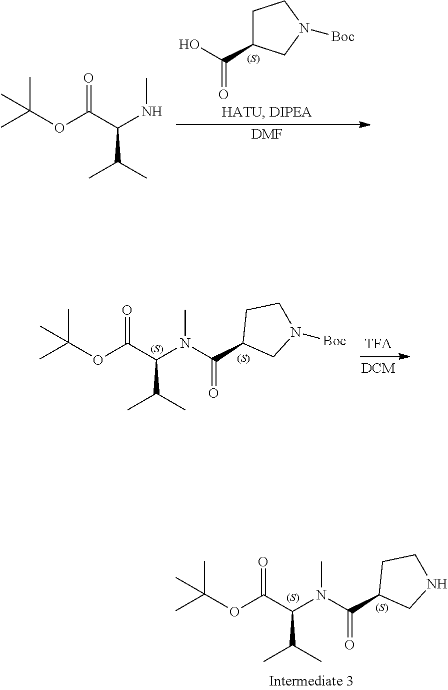

### Step 1: Synthesis of(S)-tert-butyl 3-(((S)-1-(tert-butoxy)-3-methyl-1-oxobutan-2-yl) (methyl) carbamoyl) pyrrolidine-1-carboxylate

To a mixture of(S)-1-(tert-butoxycarbonyl) pyrrolidine-3-carboxylic acid (2.2 g, 10.2 mmol) in DMF (10 mL) at room temperature was added HATU (7.8 g, 20.4 mmol) and DIPEA (5 mL). After stirring at room temperature for 10 min, tert-butyl methyl-L-valinate (3.8 g, 20.4 mmol) in DMF (10 mL) was added. The mixture was stirred at room temperature for 3 h, then diluted with DCM (40 mL) and H₂O (30 mL). The aqueous and organic layers were separated, and the organic layer was washed with H₂O (3×30 mL), brine (30 mL), dried over anhydrous Na₂SO₄ and filtered. The filtrate was concentrated under reduced pressure and the residue was purified by silica gel column chromatography to give (S)-tert-butyl 3-(((S)-1-(tert-butoxy)-3-methyl-1-oxobutan-2-yl) (methyl) carbamoyl) pyrrolidine-1-carboxylate (3.2 g, 82% yield) as an oil. LCMS (ESI) m/z: [M+Na] calcd for C₂₀H₃₆N₂O₅Na: 407.25; found 407.2.

### Step 2: Synthesis of(S)-tert-butyl 3-methyl-2-((S)—N-methylpyrrolidine-3-carboxamido) butanoate

A mixture of(S)-tert-butyl 3-(((S)-1-(tert-butoxy)-3-methyl-1-oxobutan-2-yl) (methyl) carbamoyl) pyrrolidine-1-carboxylate (3.2 g, 8.4 mmol) in DCM (13 mL) and TFA (1.05 g, 9.2 mmol) was stirred at room temperature for 5 h. The mixture was concentrated under reduced pressure to give (S)-tert-butyl 3-methyl-2-((S)—N-methylpyrrolidine-3-carboxamido) butanoate (2.0 g, 84% yield) as an oil. LCMS (ESI) m/z: [M+H] calcd for C₁₅H₂₈N₂O₃: 285.21; found 285.2.

### Intermediate 5. Synthesis of tert-butyl ((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

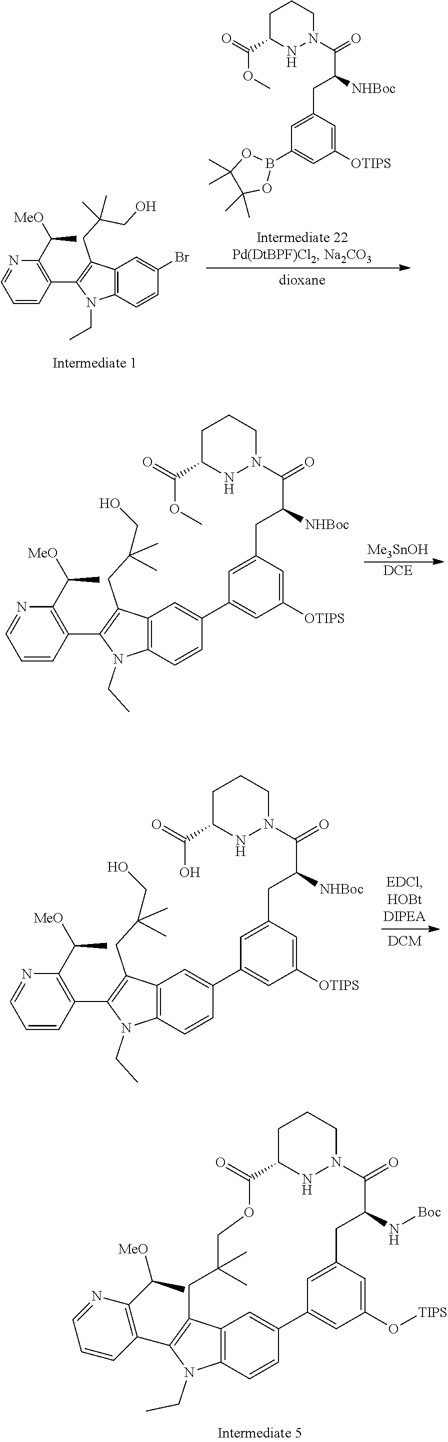

### Step 1: Synthesis of methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate

To a stirred mixture of 3-(5-bromo-1-ethyl-2-[2-[(1 S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in 1,4-dioxane (750 mL) at room temperature under an atmosphere of Ar was added Na₂CO₃ (17.9 g, 168.4 mmol), Pd(DtBPF)Cl2 (4.39 g, 6.7 mmol), and H₂O (150 mL) in portions. The mixture was heated to 85° C. and stirred for 3 h, cooled, diluted with H₂O (2 L), and extracted with EtOAc (3×1 L). The combined organic layers were washed with brine (2×500 mL), dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 72% yield) as a solid. LCMS (ESI) m/z: [M+H] calcd for C₅₂H₇₇N₅O₈Si: 928.56; found 928.8.

### Step 2: Synthesis of (3S)-1-[(2S)-2-[(tert-butoxycarbonyl) amino]-3-[3-[1-ethyl-3-(3-hydroxy-2,2-dimethylpropyl)-2-[2-[(1 S)-1-methoxyethyl]pyridin-3-yl]indol-5-yl]-5-[(triisopropylsilyl)oxy]phenyl]propanoyl]-1,2-diazinane-3-carboxylic acid

To a stirred mixture of methyl (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at room temperature was added trimethyltin hydroxide (48.7 g, 269 mmol) in portions. The mixture was heated to 65° C. and stirred for 16 h, then filtered and the filter cake washed with DCM (3×150 mL). The filtrate was concentrated under reduced pressure to give (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g, crude), which was used directly in the next step without further purification. LCMS (ESI) m/z: [M+H] calcd for C₅₁H₇₅N₅O₈Si: 914.55; found 914.6.

### Step 3: Synthesis of tert-butyl ((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred mixture of (3S)-1-[(2S)-2-[(tert-butoxycarbonyl) amino]-3-[3-[1-ethyl-3-(3-hydroxy-2,2-dimethylpropyl)-2-[2-[(1S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (400 mL, 2.3 mol), HOBT (51.7 g, 383 mmol) and EDCI (411 g, 2.1 mol) in portions. The mixture was warmed to room temperature and stirred for 16 h, then diluted with DCM (1 L), washed with brine (3×1 L), dried over anhydrous Na₂SO₄, and filtered. The filtrate was concentrated under reduced pressure and the residue was purified by silica gel column chromatography to give tert-butyl ((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (36 g, 42% yield) as a solid. LCMS (ESI) m/z: [M+H] calcd for C₅₁H₇₃N₅O₇Si: 896.54; found 896.5.

### Intermediate 6. Synthesis of tert-butyl ((6³S,4S)-12-iodo-10,10-dimethyl-5,7-dioxo-25-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate

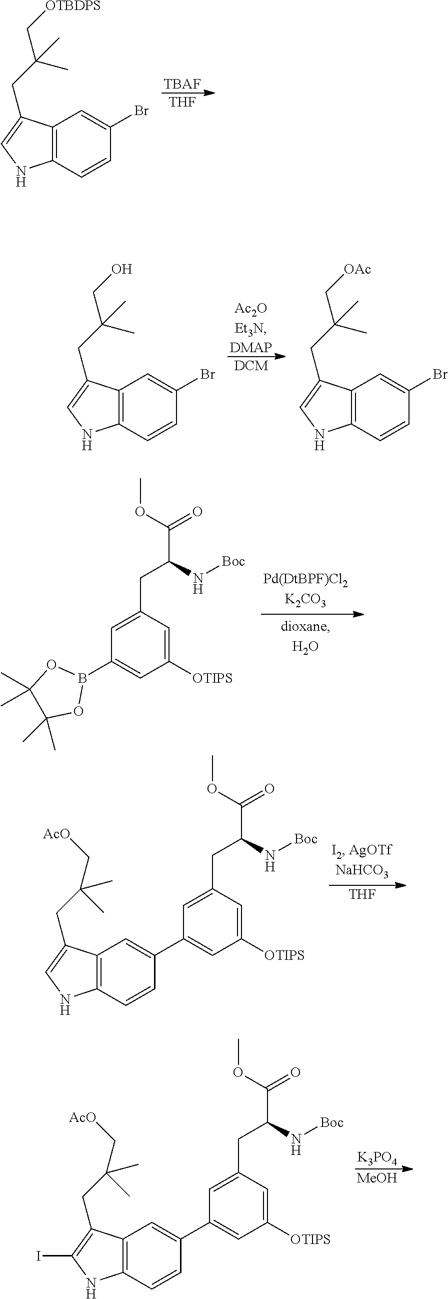

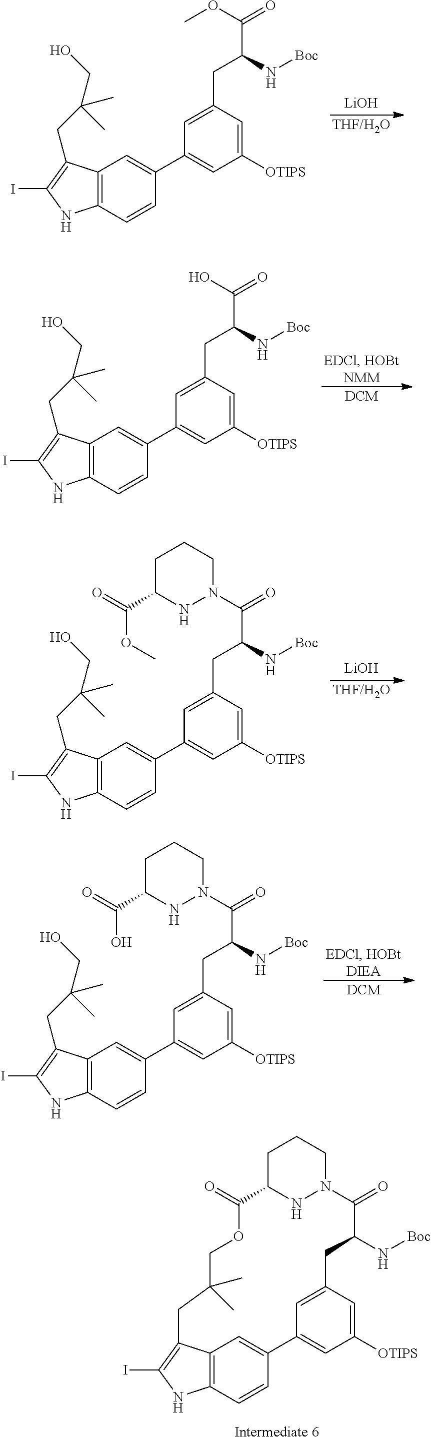

### Step 1: Synthesis of 3-(5-bromo-1H-indol-3-yl)-2,2-dimethylpropan-1-ol

This reaction was undertaken on five batches in parallel on the scale illustrated below. Into a 2 L round-bottom flask were added 5-bromo-3-[3-[(tert-butyldiphenylsilyl)oxy]-2,2-dimethylpropyl]-1H-indole (100 g, 192 mmol) and TBAF (301.4 g, 1.15 mol) in THF (1.15 L) at room temperature. The resulting mixture was heated to 50° C. and stirred for 16 h, then the mixture was concentrated under reduced pressure.

At this stage the residues from all five batches were combined, diluted with H₂O (5 L), and extracted with EtOAc (3×2 L). The combined organic layers were washed with brine (2×1.5 L), dried over anhydrous Na₂SO₄, and filtered. The filtrate was concentrated under reduced pressure and the residue was purified by silica gel column chromatography to give 3-(5-bromo-1H-indol-3-yl)-2,2-dimethylpropan-1-ol (310 g, crude) as a solid. LCMS (ESI) m/z: [M+H] calcd for C₁₃H₁₆BrNO: 282.05 and 284.05; found 282.1 and 284.1.

### Step 2: Synthesis of 3-(5-bromo-1H-indol-3-yl)-2,2-dimethylpropyl acetate

This reaction was undertaken on two batches in parallel in accordance with the procedure below. To a stirred mixture of 3-(5-bromo-1H-indol-3-yl)-2,2-dimethylpropan-1-ol (135 g, 478 mmol) and Et₃N (200 mL, 1.44 mol) in DCM (1.3 L) at 0° C. under an atmosphere of N₂ was added AC₂O (73.3 g, 718 mmol) and DMAP (4.68 g, 38.3 mmol) in portions. The resulting mixture was stirred for 10 min at 0° C., then washed with H₂O (3×2 L).

At this stage, the organic layers from both batches were combined and washed with brine (2×1 L), dried over anhydrous Na₂SO₄, and filtered. The filtrate was concentrated under reduced pressure and the residue was purified by column chromatography to give 3-(5-bromo-1H-indol-3-yl)-2,2-dimethylpropyl acetate (304 g, 88% yield) as a solid. ¹H NMR (400 MHZ, DMSO-d₆) δ 11.16-11.11 (m, 1H), 7.69 (d, J=2.0 Hz, 1H), 7.32 (d, J=8.6 Hz, 1H), 7.19-7.12 (m, 2H), 3.69 (s, 2H), 2.64 (s, 2H), 2.09 (s, 3H), 0.90 (s, 6H).

### Step 3: Synthesis of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate

This reaction was undertaken on four batches in parallel in accordance with the procedure below. Into a 2 L round-bottom flasks were added methyl (2S)-2-[(tert-butoxycarbonyl) amino]-3-[3-(4,4,5,5-tetramethyl-1,3,2-dioxaborolan-2-yl)-5-[(triisopropylsilyl)oxy]phenyl]propanoate (125 g, 216 mmol), 1,4-dioxane (1 L), H₂O (200 mL), 3-(5-bromo-1H-indol-3-yl)-2,2-dimethylpropyl acetate (73.7 g, 227 mmol), K₂CO₃ (59.8 g, 433 mmol), and Pd(DtBPF)Cl₂ (7.05 g, 10.8 mmol) at room temperature under an atmosphere of Ar. The resulting mixture was heated to 65° C. and stirred for 2 h, then diluted with H₂O (10 L) and extracted with EtOAc (3×3 L). The combined organic layers were washed with brine (2 ×2 L), dried over anhydrous Na₂SO₄, and filtered. The filtrate was concentrated under reduced pressure.

At this point the residue from all four batches was combined and purified by column chromatography to give methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (500 g, 74% yield) as an oil. LCMS (ESI) m/z: [M+Na] calcd for C₃₉H₅₈N₂O₇SiNa: 717.39; found 717.3.

### Step 4: Synthesis of methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate

This reaction was undertaken on three batches in parallel in accordance with the procedure below. To a stirred mixture of methyl (2S)-3-(3-[3-[3-(acetyloxy)-2,2-dimethylpropyl]-1H-indol-5-yl]-5-[(triisopropylsilyl)oxy]phenyl)-2-[(tert-butoxycarbonyl) amino]propanoate (150 g, 216 mmol) and NaHCO₃ (21.76 g, 259 mmol) in THF (1.5 L) was added AgOTf (66.5 g, 259 mmol) in THE dropwise at 0° C. under an atmosphere of nitrogen. 12 (49.3 g, 194 mmol) in THF was added dropwise over 1 h at 0° C. and the resulting mixture was stirred for an additional 10 min at 0° C. The combined experiments were diluted with aqueous Na₂S₂O₃ (5 L) and extracted with EtOAc (3×3 L). The combined organic layers were washed with brine (2×1.5 L), dried over anhydrous Na₂SO₄, filtered, and concentrated under reduced pressure to a residue.

At this stage, the residue from all three batches was combined and purified by column chromatography to give methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (420 g, 71% yield) as an oil. LCMS (ESI) m/z: [M+Na] calcd for C₃₉H₅₇IN₂O₇SiNa: 843.29; found 842.9.

### Step 5: Synthesis of methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate

This reaction was undertaken on three batches in parallel in accordance with the procedure below. To a 2 L round-bottom flask were added methyl (2S)-3-(3-[3-[3-(acetyloxy)-2,2-dimethylpropyl]-2-iodo-1H-indol-5-yl]-5-[(triisopropylsilyl)oxy]phenyl)-2-[(tert-butoxycarbonyl) amino]propanoate (140 g, 171 mmol), MeOH (1.4 L), and K₃PO₄ (108.6 g, 512 mmol) at 0° C. The mixture was warmed to room temperature and stirred for 1 h, then the combined experiments were diluted with H₂O (9 L) and extracted with EtOAc (3×3 L). The combined organic layers were washed with brine (2×2 L), dried over anhydrous Na₂SO₄, filtered, and the filtrate was concentrated under reduced pressure.

At this stage the residue from all three batches was combined to give methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (438 g, crude) as a solid. LCMS (ESI) m/z: [M+Na] calcd for C₃₇H₅₅IN₂O₆SiNa: 801.28; found 801.6.

### Step 6: Synthesis of (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid

This reaction was undertaken on three batches in parallel in accordance with the procedure below. To a stirred mixture of methyl (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoate (146 g, 188 mmol) in THF (1.46 L) was added LiOH (22.45 g, 937 mmol) in H₂O (937 mL) dropwise at 0° C. The resulting mixture was warmed to room temperature and stirred for 1.5 h [note: LCMS showed 15% de-TIPS product]. The mixture was acidified to pH 5 with 1M HCl (1M) and the combined experiments were extracted with EtOAc (3×3 L). The combined organic layers were washed with brine (2×2 L), dried over anhydrous Na₂SO₄, filtered, and the filtrate was concentrated under reduced pressure.

At this stage the residue from all three batches was combined to give (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (402 g, crude) as a solid. LCMS (ESI) m/z: [M+Na] calcd for C₃₆H₅₃IN₂O₈SiNa: 787.26; found 787.6.

### Step 7: Synthesis of methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate

To a stirred mixture of (2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoic acid (340 g, 445 mmol) and methyl (3S)-1,2-diazinane-3-carboxylate (96.1 g, 667 mmol) in DCM (3.5 L) was added NMM (225 g, 2.2 mol), EDCI (170 g, 889 mmol), and HOBt (12.0 g, 88.9 mmol) portionwise at 0° C. The mixture was warmed to room temperature and stirred for 16 h, then washed with H₂O (3×2.5 L), brine (2×1 L), dried over anhydrous Na₂SO₄, and filtered. The filtrate was concentrated under reduced pressure and the residue was purified by column chromatography to give methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (310 g, 62% yield) as an oil. LCMS (ESI) m/z: [M+H] calcd for C₄₂H₆₃IN₄O₇Si: 891.36; found 890.8.

### Step 8: Synthesis of (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid

This reaction was undertaken on three batches in parallel in accordance with the procedure below. To a stirred mixture of methyl (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylate (85.0 g, 95.4 mmol) in THF (850 mL) was added LiOH (6.85 g, 286 mmol) in H₂O (410 mL) dropwise at 0° C. under an atmosphere of N₂. The mixture was stirred at 0° C. for 1.5 h [note: LCMS showed 15% de-TIPS product], then acidified to pH 5 with 1M HCl

At this stage the mixtures from all three batches was combined and extracted with EtOAc (3×2 L). The combined organic layers were washed with brine (2×1.5 L), dried over anhydrous Na₂SO₄, filtered, and the filtrate was concentrated under reduced pressure to give (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (240 g, crude) as a solid. LCMS (ESI) m/z: [M+H] calcd for C₄₁H₆₁IN4O₇Si: 877.35; found 877.6.

### Step 9: Synthesis of tert-butyl ((6³S,4S)-12-iodo-10, 10-dimethyl-5,7-dioxo-25-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate

This reaction was undertaken on two batches in parallel in accordance with the procedure below. To a stirred mixture of (3S)-1-[(2S)-2-[(tert-butoxycarbonyl) amino]-3-[3-[3-(3-hydroxy-2,2-dimethylpropyl)-2-iodo-1H-indol-5-yl]-5-[(triisopropylsilyl)oxy]phenyl]propanoyl]-1,2-diazinane-3-carboxylic acid (120 g, 137 mmol) in DCM (6 L) was added DIPEA (357 mL, 2.05 mol), EDCI (394 g, 2.05 mol), and HOBT (37 g, 274 mmol) in portions at 0° C. under an atmosphere of N₂. The mixture was warmed to room temperature and stirred overnight.

At this stage the solutions from both batches were combined and washed with H₂O (3×6 L), brine (2×6 L), dried over anhydrous Na₂SO₄, and filtered. The filtrate was concentrated under reduced pressure and the residue was purified by column chromatography to give tert-butyl ((6³S,4S)-12-iodo-10, 10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (140 g, 50% yield) as a solid. LCMS (ESI) m/z: [M+H] calcd for C₄₁H₅₉IN₄O₆Si: 859.33; found 858.3.

### Intermediate 7. Synthesis of (6³S,4S)-4-amino-1¹-ethyl-25-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

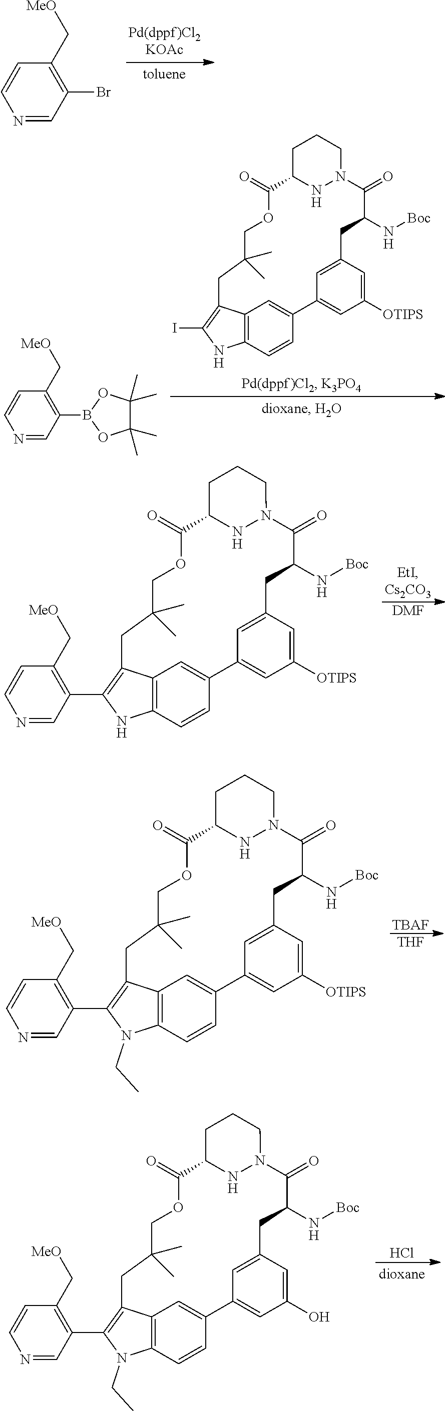

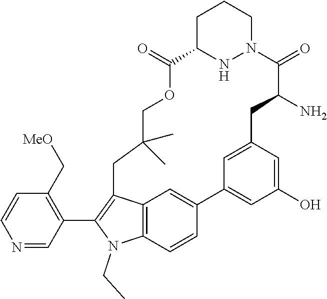 [SMILES: CCn1c(-c2cnccc2COC)c2c3cc(ccc31)-c1cc(O)cc(c1)C[C@H](N)C(=O)N1CCC[C@H](N1)C(=O)OCC(C)(C)C2]

### Step 1: Synthesis of 4-(methoxymethyl)-3-(4,4,5,5-tetramethyl-1,3,2-dioxaborolan-2-yl) pyridine

To a mixture of 3-bromo-4-(methoxymethyl)pyridine (1.0 g, 5.0 mmol), 4,4,5,5-tetramethyl-2-(tetramethyl-1,3,2-dioxaborolan-2-yl)-1,3,2-dioxaborolane (1.51 g, 5.9 mmol) and KOAc (1.21 g, 12.3 mmol) in toluene (10 mL) at room temperature under an atmosphere of Ar was added Pd(dppf)Cl₂ (362 mg, 0.5 mmol). The mixture was heated to 110° C. and stirred overnight, then concentrated under reduced pressure to give 4-(methoxymethyl)-3-(4,4,5,5-tetramethyl-1,3,2-dioxaborolan-2-yl) pyridine, which was used directly in the next step directly without further purification. LCMS (ESI) m/z: [M+H] calcd for C₁₃H₂₀BNO₃: 250.16; found 250.3.

### Step 2: Synthesis of give tert-butyl ((6³S,4S)-12-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate

To a mixture of 4-(methoxymethyl)-3-(4,4,5,5-tetramethyl-1,3,2-dioxaborolan-2-yl) pyridine (290 mg, 1.16 mmol), K₃PO₄ (371 mg, 1.75 mmol) and tert-butyl ((6³S,4S)-12-iodo-10, 10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (500 mg, 0.58 mmol) in 1,4-dioxane (5 mL) and H₂O (1 mL) at room temperature under an atmosphere of Ar was added Pd(dppf)Cl₂ (43 mg, 0.06 mmol). The mixture was heated to 70° C. and stirred for 2 h, then H₂O was added and the mixture extracted with EtOAc (2×10 mL). The combined organic layers were washed with brine (10 mL), dried over anhydrous Na₂SO₄, and filtered. The filtrate was concentrated under reduced pressure and the residue was purified by silica gel column chromatography to give tert-butyl ((6³S,4S)-12-(4-(methoxymethyl) pyridin-3-yl)-10,10-dimethyl-5,7-dioxo-2⁵-((triisopropylsilyl) oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (370 mg, 74% yield) as a foam. LCMS (ESI) m/z: [M+H] calcd for C₄₈H₆₇N₅₀₇Si: 854.49; found 854.6.

### Step 3: Synthesis of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate

A mixture of tert-butyl ((6³S,4S)-12-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (350 mg, 0.41 mmol), CS₂CO₃ (267 mg, 0.82 mmol), and EtI (128 mg, 0.82 mmol) in DMF (4 mL) was stirred at 35° C. overnight. H₂O was added and the mixture was extracted with EtOAc (2×15 mL). The combined organic layers were washed with brine (15 mL), dried over anhydrous Na₂SO₄, and filtered. The filtrate was concentrated under reduced pressure and the residue was purified by silica gel column chromatography to give tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (350 mg, 97% yield) as an oil. LCMS (ESI) m/z: [M+H] calcd for C₅₀H₇₁N₅O₇Si: 882.52; found 882.6.

### Step 4: Synthesis of tert-butyl ((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

A mixture of tert-butyl ((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-11-H-8-oxa-1 (5,3)-indola-6 (1,3)-pyridazina-2 (1,3)-benzenacycloundecaphane-4-yl) carbamate (350 mg, 0.4 mmol) and 1M TBAF in THF (0.48 mL, 0.480 mmol) in THF (3 mL) at 0° C. under an atmosphere of Ar was stirred for 1 h.

The mixture was concentrated under reduced pressure and the residue was purified by silica gel column chromatography to give tert-butyl ((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (230 mg, 80% yield) as an oil. LCMS (ESI) m/z: [M+H] calcd for C₄₁H₅₁N₅O₇: 726.39; found 726.6.

### Step 5: Synthesis of (6³S,4S)-4-amino-1¹-ethyl-25-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a mixture of tert-butyl N-[(8S, 14S)-22-ethyl-4-hydroxy-21-[4-(methoxymethyl)pyridin-3-yl]-18,18-dimethyl-9, 15-dioxo-16-oxa-10,22,28-triazapentacyclo[18.5.2.1\hat{ }[2,6]0.1\hat{ }[10,14]0.0\hat{ }[23,27]nonacosa-1 (26),2,4,6 (29),20,23 (27),24-heptaen-8-yl]carbamate (200 mg, 0.28 mmol) in 1,4-dioxane (2 mL) at 0° C. under an atmosphere of Ar was added 4M HCl in 1,4-dioxane (2 mL, 8 mmol). The mixture was allowed to warm to room temperature and was stirred overnight, then concentrated under reduced pressure to give (6³S,4S)-4-amino-1¹-ethyl-25-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (200 mg). LCMS (ESI) m/z: [M+H] calcd for C₃₆H₄₃N₅O₅: 626.34; found 626.5.

### Intermediate 8. Synthesis of (6³S,4S)-4-amino-1¹-ethyl-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

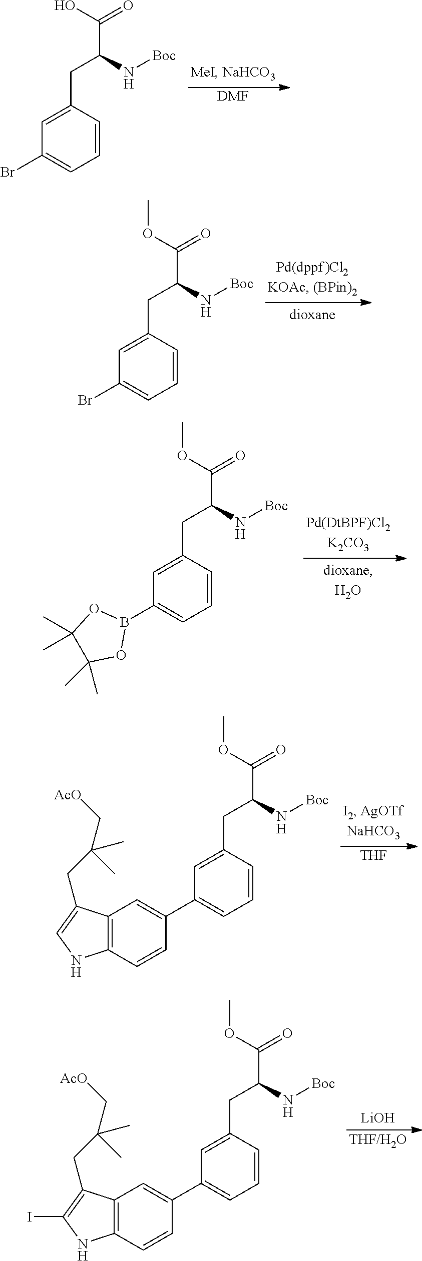

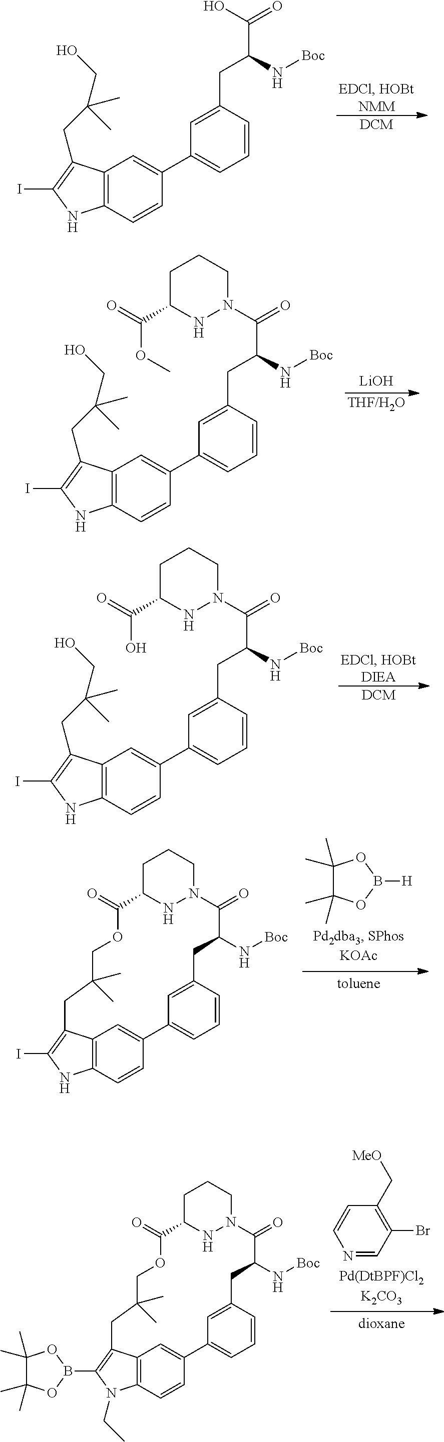

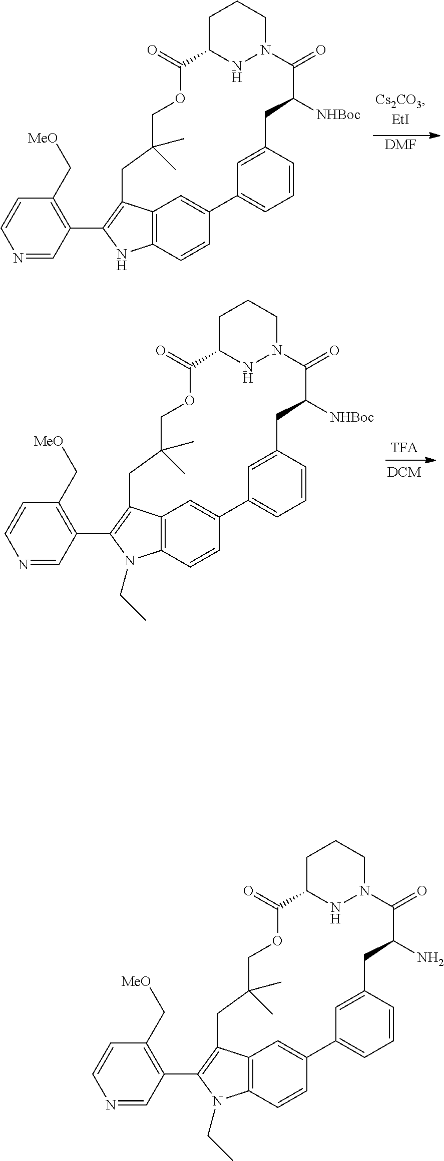

### Step 1: Synthesis of methyl(S)-3-(3-bromophenyl)-2-((tert-butoxycarbonyl) amino) propanoate

To a solution of (2S)-3-(3-bromophenyl)-2-[(tert-butoxycarbonyl) amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel chromatography (13% EtOAc/pet. ether) to afford the desired product (109 g, crude). LCMS (ESI) m/z: [M+Na] calcd for C₁₅H₂₀BrNO₄: 380.05; found 380.0.

### Step 2: Synthesis of methyl(S)-2-((tert-butoxycarbonyl) amino)-3-(3-(4,4,5,5-tetramethyl-1,3,2-dioxaborolan-2-yl)phenyl) propanoate

To a stirred solution of methyl (2S)-3-(3-bromophenyl)-2-[(tert-butoxycarbonyl) amino]propanoate (108 g, 301.5 mmol) and bis (pinacolato)diboron (99.53 g, 391.93 mmol) in 1,4-dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel chromatography (5% EtOAc/pet. ether) to afford the desired product (96 g, 78.6% yield). LCMS (ESI) m/z: [M+Na] calcd for C₂₁H₃₂BNO₆: 428.22; found 428.1.

### Step 3: Synthesis of methyl(S)-3-(3-(3-(3-acetoxy-2,2-dimethylpropyl)-1H-indol-5-yl)phenyl)-2-((tert-butoxycarbonyl) amino) propanoate

To a mixture of methyl (2S)-2-[(tert-butoxycarbonyl) amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in 1,4-dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel chromatography (20% EtOAc/pet. ether) to afford the desired product (130 g, crude). LCMS (ESI) m/z: [M+H] calcd for C₃₀H₃₈N₂O₆: 523.28; found 523.1.

### Step 4: Synthesis of methyl(S)-3-(3-(3-(3-acetoxy-2,2-dimethylpropyl)-2-iodo-1H-indol-5-yl)phenyl)-2-((tert-butoxycarbonyl) amino) propanoate

To a solution of methyl (2S)-3-(3-[3-[3-(acetyloxy)-2,2-dimethylpropyl]-1H-indol-5-yl]phenyl)-2-[(tert-butoxycarbonyl) amino]propanoate (95.0 g, 181.8 mmol) and iodine (36.91 g, 145.41 mmol) in THF (1 L) at −10° C. was added AgOTf (70.0 g, 272.7 mmol) and NaHCO₃ (22.9 g, 272.65 mmol). The reaction mixture was stirred for 30 min and was then quenched by the addition of sat. Na₂S₂O₃ (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel chromatography (50% EtOAc/pet. ether) to afford the desired product (49.3 g, 41.8% yield). LCMS (ESI) m/z: [M+H] calcd for C₃₀H₃₇IN₂O₆: 649.18; found 649.1.

### Step 5: Synthesis of(S)-2-((tert-butoxycarbonyl) amino)-3-(3-(3-(3-hydroxy-2,2-dimethylpropyl)-2-iodo-1H-indol-5-yl)phenyl) propanoic acid

To a solution of methyl (2S)-3-(3-[3-[3-(acetyloxy)-2,2-dimethylpropyl]-2-iodo-1H-indol-5-yl]phenyl)-2-[(tert-butoxycarbonyl) amino]propanoate (60 g, 92.5 mmol) in THF (600 mL) was added a solution of LiOH·H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with sat. brine (2×500 mL), dried over Na₂SO₄, filtered, and concentrated under reduced pressure to afford the desired product (45 g, 82.1% yield). LCMS (ESI) m/z: [M+Na] calcd for C₂₇H₃₃IN₂O₅: 615.13; found 615.1.

### Step 6: Synthesis of methyl(S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylate

To a solution of (2S)-2-[(tert-butoxycarbonyl) amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBT (2.05 g, 15.19 mmol), and EDCI (19.41 g, 101.27 mmol). The reaction mixture was stirred overnight and then the mixture was washed with sat. NH₄Cl (2×200 mL) and sat. brine (2×200 mL), and the mixture was dried over Na₂SO₄, filtered, and concentrated under reduced pressure to afford the desired product (14 g, 38.5% yield). LCMS (ESI) m/z: [M+H] calcd for C₃₃H₄₃IN₄O₆: 718.23; found 719.4.

### Step 7: Synthesis of(S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylic acid

To a solution of methyl(S)-1-((S)-2-((tert-butoxycarbonyl) amino)-3-(3-(3-(3-hydroxy-2,2-dimethylpropyl)-2-iodo-1H-indol-5-yl)phenyl) propanoyl) hexahydropyridazine-3-carboxylate (92 g, 128.0 mmol) in THF (920 mL) at 0° C. was added a solution of LiOH·H₂O (26.86 g, 640.10 mmol) in H₂O (640 mL). The reaction mixture was stirred for 2 h and was then concentrated under reduced pressure to afford the desired product (90 g, crude). LCMS (ESI) m/z: [M+H] calcd for C₃₂H₄₁IN₄O₆: 705.22; found 705.1).

### Step 8: Synthesis of tert-butyl ((6³S,4S)-12-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of (3S)-1-[(2S)-2-[(tert-butoxycarbonyl) amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel chromatography (50% EtOAc/pet. ether) to afford the desired product (70 g, 79.8% yield). LCMS (ESI) m/z: [M+H] calcd for C₃₂H₃₉IN₄O₅: 687.21; found 687.1.

### Step 9: Synthesis of tert-butyl ((6³S,4S)-12-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-12-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (22.0 g, 32.0 mmol) in toluene (300.0 mL) was added Pd₂(dba)₃ (3.52 g, 3.85 mmol), S-Phos (3.95 g, 9.61 mmol), and KOAc (9.43 g, 96.13 mmol) followed by 4,4,5,5-tetramethyl-1,3,2-dioxaborolane (26.66 g, 208.3 mmol), dropwise. The resulting solution was heated to 60° C. and stirred for 3 h. The reaction mixture was then cooled to room temperature, filtered, the filter cake was washed with EtOAc, and the filtrate was concentrated under reduced pressure. The residue was purified by normal phase chromatography to afford the desired product (22 g, 90% yield) as a solid. LCMS (ESI) m/z: [M+H] calcd for C₃₈H₅₁BN₄O₇: 687.39; found 687.3.

### Step 10: Synthesis of tert-butyl ((6³S,4S)-12-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a mixture of tert-butyl ((6³S,4S)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (3.0 g, 4.37 mmol) and 3-bromo-4-(methoxymethyl)pyridine (1.766 g, 8.74 mmol) in dioxane/H₂O (5/1) at 60° C. was added K₂CO₃ (2.415 g, 17.48 mmol) and Pd(DTBPF)Cl₂ (0.5695 g, 0.874 mmol). The reaction mixture was stirred for 4 h. The reaction mixture was cooled to room temperature and was extracted with EtOAc (300 mL). The solution was washed with brine (3×100 mL), dried over Na₂SO₄, filtered, and concentrated under reduced pressure. The residue was purified by normal phase chromatography (50% EtOAc/pet. ether) to afford the desired product (1.96 g, 65.8% yield) as a solid. LCMS (ESI) m/z: [M+H] calcd for C₃₉H₄₇N₅O₆: 682.36; found 682.7.

### Step 11: Synthesis of tert-butyl ((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-12-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.96 g, 2.88 mmol) and ethyl iodide (0.347 mL, 4.31 mmol) in DMF (20.0 mL) was added Cs₂CO₃ (2.342 g, 7.19 mmol). The resulting mixture was stirred at room temperature for 5 h and then diluted with EtOAc (200 mL). The mixture was washed with H₂O (3×100 mL), dried over Na₂SO₄, filtered, and concentrated under reduced pressure. The residue was purified by normal phase chromatography (50% EtOAc/pet. ether) to afford the desired product (1.24 g, 61% yield) as a solid. LCMS (ESI) m/z: [M+H] calcd for C₄₁H₅₁N₅O₆: 710.39; found 710.7.

### Step 12: Synthesis of (6³S,4S)-4-amino-1¹-ethyl-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a solution of tert-butyl ((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.09 g, 1.54 mmol) in DCM (1.5 mL) at 0° C. was added TFA (1.50 mL). The reaction mixture was stirred for 1 h, concentrated under reduced pressure, and then azeotroped with toluene (3×20 mL) to afford the desired crude product (1.09 g) as a solid. LCMS (ESI) m/z: [M+H] calcd for C₃₆H₄₃N₅O₄: 610.34; found 610.4.

### Intermediate 9. Synthesis of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

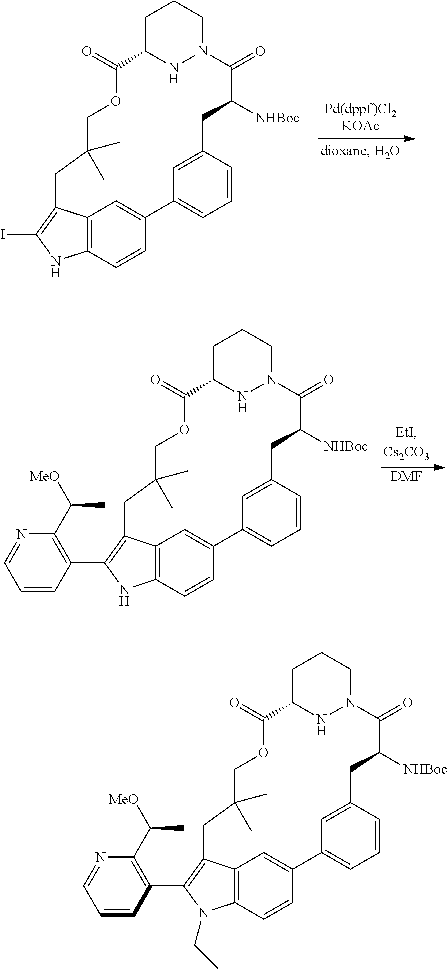

### Step 1: Synthesis of tert-butyl ((6³S,4S)-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-12-iodo-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (13 g, 18.93 mmol) and 2-[(1S)-1-methoxyethyl]-3-(4,4,5,5-tetramethyl-1,3,2-dioxaborolan-2-yl) pyridine (14.95 g, 56.8 mmol) in dioxane (130 mL) and H₂O (26 mL) was added K₂CO₃ (5.23 g, 37.9 mmol) and Pd(dppf)Cl₂ (1.39 g, 1.89 mmol). The reaction mixture was stirred for 4 h at 70° C. The mixture was cooled to room temperature, filtered, and washed with EtOAc (3×100 mL). The filtrate was washed with brine (2×100 mL), dried over Na₂SO₄, filtered, and concentrated under reduced pressure. The residue was purified by prep-TLC(10% MeOH/DCM) to afford the desired product (21 g, 85.3% yield). LCMS (ESI) m/z: [M+H] calcd for C₄₀H₄₉N₅O₆: 696.38; found 696.4.

### Step 2: Synthesis of tert-butyl ((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (20 g, 28.7 mmol) and CS₂CO₃ (18.7 g, 57.5 mmol) in DMF (150 mL) at 0° C. was added a solution of ethyl iodide (13.45 g, 86.22 mmol) in DMF (50 mL). The resulting mixture was stirred overnight at 35° C. and was then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel chromatography (10%->50% EtOAc/pet. ether) to afford the desired product (4.23 g, 18.8% yield) and the atropisomer (5.78 g, 25.7% yield). LCMS (ESI) m/z: [M+H] calcd for C₄₂H₅₃N₅O₆: 724.41; found 724.4.

### Intermediate 10. Synthesis of (2S)—N-((63,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide

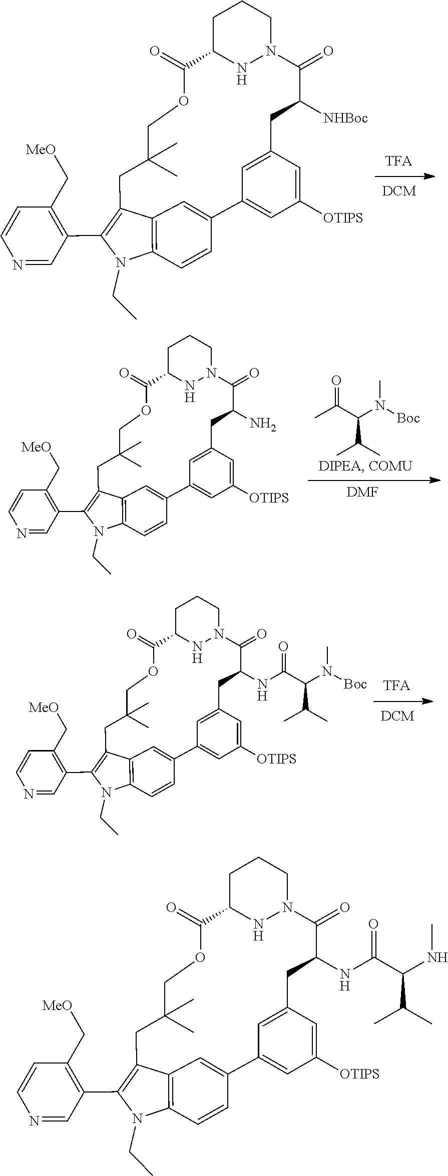

### Step 1: Synthesis of (6³S,4S)-4-amino-1¹-ethyl-1²-(4-(methoxymethyl)pyridin-3-yl)-10,10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

A mixture of tert-butyl ((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (880 mg, 1.2 mmol), DCM (10 mL), and TFA (5 mL) was stirred at 0° C. for 30 min. The mixture was concentrated under reduced pressure to afford the desired product, which was used directly in the next step without further purification. LCMS (ESI) m/z: [M+H] calcd for C₄₅H₆₃N₅O₅Si: 782.47; found 782.7.

### Step 2: Synthesis of tert-butyl ((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate

To a mixture of (6³S,4S)-4-amino-1¹-ethyl-1²-(4-(methoxymethyl)pyridin-3-yl)-10,10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (880 mg, 1.13 mmol) and N-(tert-butoxycarbonyl)-N-methyl-L-valine (521 mg, 2.3 mmol) in DMF (8.8 mL) at 0° C. was added DIPEA (1.95 mL, 11.3 mmol) and COMU (88 mg, 0.21 mmol). The mixture was stirred at 0° C. for 30 min, then diluted with H₂O (100 mL) and extracted with EtOAc (3×100 mL). The combined organic layers were washed with brine (3×100 mL), dried over anhydrous Na₂SO₄, and filtered. The filtrate was concentrated under reduced pressure and the residue was purified by prep-TLC to afford the desired product (1 g, 89% yield) as a solid. LCMS (ESI) m/z: [M+H] calcd for C₅₆H₈₂N₆O₈Si: 995.61; found 995.5.

### Step 3: Synthesis of (2S)—N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide

A mixture of tert-butyl ((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (1.0 g, 1.0 mmol), DCM (10 mL) and TFA (5 mL) was stirred for 30 min. The mixture was concentrated under reduced pressure and the residue was basified to PH ˜8 with sat. NaHCO₃, then extracted with EtOAc (3×10 mL). The combined organic layers were washed with brine (3×10 mL), dried over Na₂SO₄, filtered, and the filtrate concentrated under reduced pressure to afford the desired product (880 mg, 98% yield) as a solid. LCMS (ESI) m/z: [M+H] calcd for C₅₁H₇₄N₆O₆Si: 895.55; found 895.5.

### Intermediate 11. Synthesis of (2S)—N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide

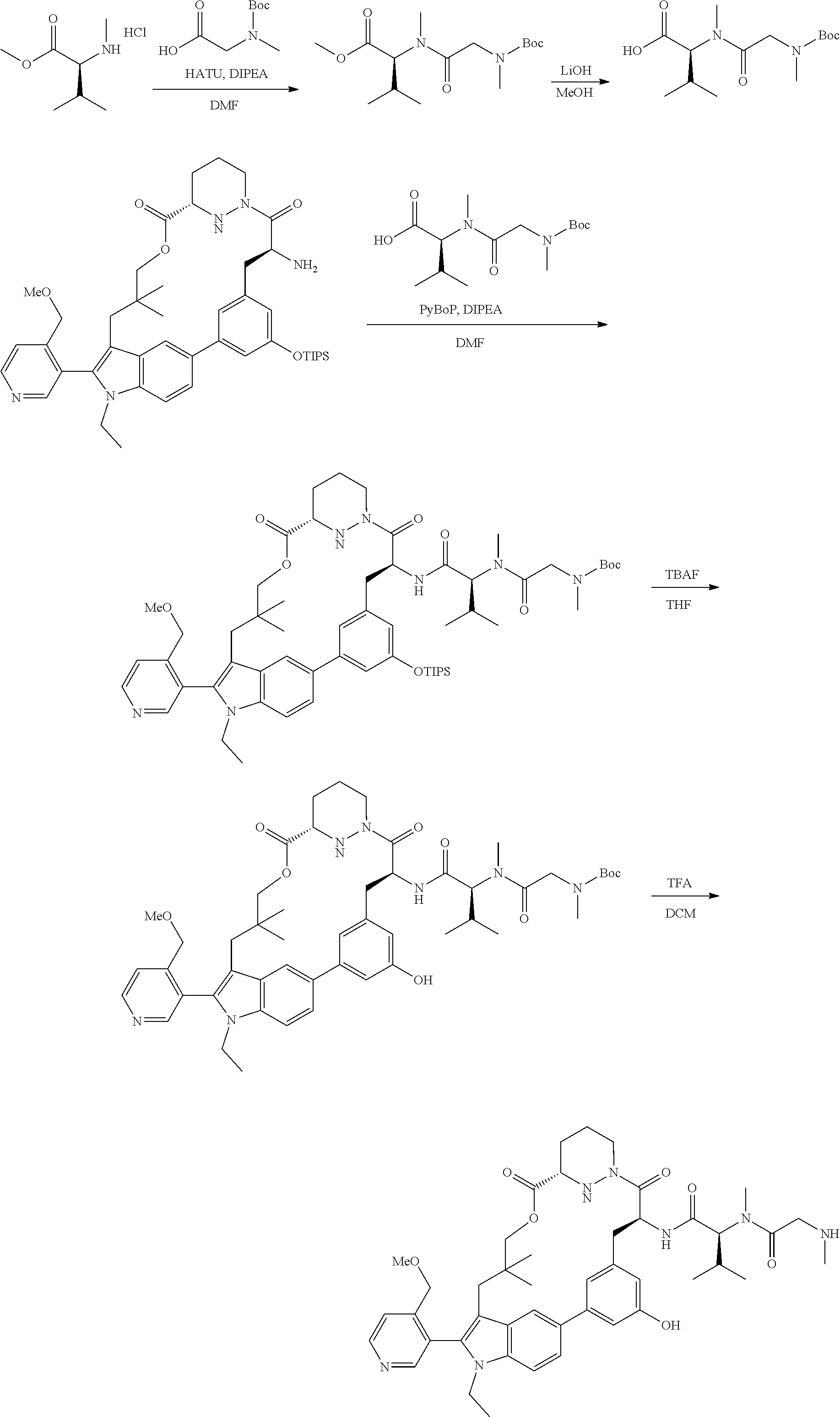

### Step 1: Synthesis of methyl N—(N-(tert-butoxycarbonyl)-N-methylglycyl)-N-methyl-L-valinate

To a solution of methyl methyl-L-valinate hydrochloride (2.0 g, 11.01 mmol) and N-(tert-butoxycarbonyl)-N-methylglycine (3.12 g, 16.51 mmol) in DMF (60.0 mL) at 0° C. was added DIPEA (9.58 mL, 55.01 mmol) and HATU (8.37 g, 22.02 mmol). The reaction mixture was stirred overnight and was then quenched with H₂O (100 mL). The mixture was extracted with EtOAc (3×100 mL) and the combined organic layers were washed with brine (100 mL), dried over Na₂SO₄, and concentrated under reduced pressure. The residue was purified by reverse phase chromatography (40→60% MeCN/H₂O) to afford the desired product (2.9 g, 83% yield) as an oil. LCMS (ESI) m/z: [M+H] calcd for C₁₅H₂₈N₂O₅: 317.21; found 317.4.

### Step 2: Synthesis of N—(N-(tert-butoxycarbonyl)-N-methylglycyl)-N-methyl-L-valine

To a solution of methyl N—(N-(tert-butoxycarbonyl)-N-methylglycyl)-N-methyl-L-valinate (3.70 g, 11.69 mmol) in THF (37.0 mL) was added a solution of LiOH·H₂O (1.96 g, 46.71 mmol) in H₂O (47.0 mL). The reaction mixture was stirred for 4 h, and then 1M HCl was added until the pH was adjusted to 5. The resulting solution was extracted with EtOAc (3×100 mL) and the combined organic layers were washed with brine (3×50 mL), dried over Na₂SO₄, and concentrated under reduced pressure. The residue was purified by reverse phase chromatography (60→60% MeCN/H₂O) to afford the desired product (1.47 g, 41.6% yield) as a solid. LCMS (ESI) m/z: [M+H] calcd for C₁₄H₂₆N₂O₅: 303.19; found 303.4.

### Step 3: Synthesis of tert-butyl (2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate

To a solution of (6³S,4S)-4-amino-1¹-ethyl-1²-(4-(methoxymethyl)pyridin-3-yl)-10, 10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (300.0 mg, 0.384 mmol) and N—(N-(tert-butoxycarbonyl)-N-methylglycyl)-N-methyl-L-valine (173.9 mg, 0.575 mmol) in DMF (3.0 mL) at 0° C. was added DIPEA (0.534 mL, 3.069 mmol) and PyBOP (399.2 mg, 0.767 mmol). The reaction mixture was stirred for 2 h and was then diluted with H₂O (30 mL). The resulting mixture was extracted with EtOAc (3×20 mL) and the combined organic layers were washed with brine (3×20 mL), dried over Na₂SO₄, filtered, and concentrated under reduced pressure. The residue was purified by normal phase chromatography (25% EtOAc/pet. ether) to afford the desired product (300 mg, 73% yield) as a solid. LCMS (ESI) m/z: [M+H] calcd for C₅₉H₈₇N₇O₉Si: 1066.64; found 1067.4.

### Step 4: Synthesis of tert-butyl (2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate

To a solution of tert-butyl (2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate (355.0 mg) in THF (4.0 mL) at 0° C. was added TBAF (1.0 mL). The reaction mixture was stirred for 1 h and was then concentrated under reduced pressure. The residue was purified by normal phase chromatography (25% EtOAc/pet. ether) to afford the desired product (280 mg, 92% yield) as a solid. LCMS (ESI) m/z: [M+H] calcd for C₅₀H₆₇N₇O₉: 910.51; found 911.0.

### Step 5: Synthesis of (2S)—N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide

To a solution of tert-butyl (2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate (150.0 mg, 0.165 mmol) in DCM (2.0 mL) at 0° C. was added TFA (0.70 mL). The reaction mixture was stirred for 1 h and was then concentrated under reduced pressure to afford the desired crude product (150 mg) as a solid. LCMS (ESI) m/z: [M+H] calcd for C₄₅H₅₉N₇O₇: 810.46; found 810.4.

### Intermediate 12. Synthesis of (3S)—N-((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

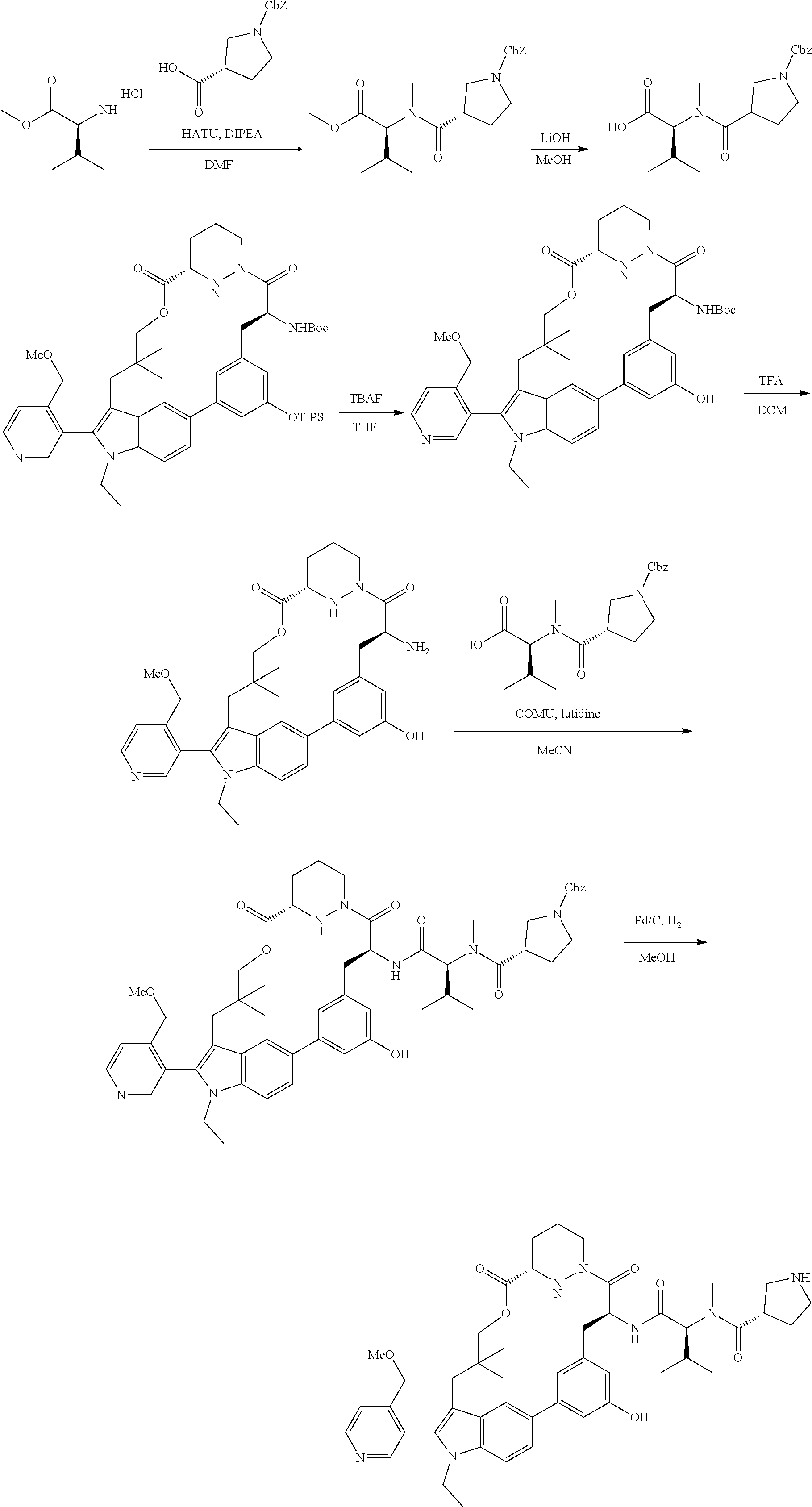

### Step 1: Synthesis of benzyl(S)-3-(((S)-1-methoxy-3-methyl-1-oxobutan-2-yl) (methyl) carbamoyl) pyrrolidine-1-carboxylate

To a solution of methyl methyl-L-valinate hydrochloride (2.0 g, 13.8 mmol) and(S)-1-((benzyloxy) carbonyl) pyrrolidine-3-carboxylic acid (4.12 mg, 16.5 mmol) in DMF (20.0 mL) at 0° C. was added DIPEA (12 mL, 68.870 mmol). The reaction mixture was stirred for 0.5 h, and then HATU (7.856 mg, 20.66 mmol) was added. The resulting mixture was warmed to room temperature and stirred for 1 h. The reaction mixture was then diluted with EtOAc (800 mL) and was washed with sat. NH₄Cl (500 mL) and brine (3×350 mL). The combined organic layers were dried over Na₂SO₄, filtered, and concentrated under reduced pressure. The residue was purified by normal phase chromatography (0→80% EtOAc/pet. ether) to afford the desired product (3.8 g, 73% yield) as an oil. LCMS (ESI) m/z: [M+H] calcd for C₂₀H₂₈N₂O₅: 377.21; found 377.2.

### Step 2: Synthesis of N—((S)-1-((benzyloxy) carbonyl) pyrrolidine-3-carbonyl)-N-methyl-L-valine

To a solution of benzyl(S)-3-(((S)-1-methoxy-3-methyl-1-oxobutan-2-yl) (methyl) carbamoyl) pyrrolidine-1-carboxylate (1.125 g, 2.99 mmol) in MeOH (10.0 mL) was added a solution of LiOH (180.0 mg, 7.52 mmol) in H₂O (2 mL). The reaction mixture was stirred for 4 h and was then quenched with sat. aq. NH₄Cl. The mixture with extracted with EtOAc (3×60 mL) and the combined organic layers were concentrated under reduced pressure to afford the desired product. LCMS (ESI) m/z: [M+H] calcd for C₁₉H₂₆N₂O₅: 363.19; found 363.2.

### Step 3: Synthesis of tert-butyl ((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (1.70 g, 1.93 mmol) in THF (20 mL) at 0° C. was added TBAF (755.7 mg, 2.89 mmol). The reaction mixture was stirred for 2 h and was then quenched with H₂O (200 mL). The resulting mixture was extracted with EtOAc (3×200 mL) and the combined organic layers were washed with brine (3×200 mL), dried over Na₂SO₄, filtered, and concentrated under reduced pressure. The residue was purified by normal phase chromatography (17% EtOAc/pet. ether) to afford the desired product (1.1 g, 70% yield) as a solid. LCMS (ESI) m/z: [M+H] calcd for C₄₁H₅₁N₅O₇: 726.39; found 726.7.

### Step 4: Synthesis of (6³S,4S)-4-amino-1¹-ethyl-25-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a solution of tert-butyl ((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (500.0 mg, 0.689 mmol) in DCM (10.0 mL) at 0° C. was added TFA (0.527 mL, 6.888 mmol). The resulting mixture was stirred for 1 h and then was concentrated under reduced pressure to afford the desired crude product (500 mg) as a solid. LCMS (ESI) m/z: [M+H] calcd for C₃₆H₄₃N₅O₅: 626.34; found 626.4.

### Step 5: Synthesis of benzyl (3S)-3-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) pyrrolidine-1-carboxylate

To a solution of N—((S)-1-((benzyloxy) carbonyl) pyrrolidine-3-carbonyl)-N-methyl-L-valine (676.4 mg, 6.31 mmol) in MeCN (10.0 mL) at 0° C. was added COMU (432.5 mg, 1.01 mmol). The reaction mixture was stirred for 5 min followed by the addition of (6³S,4S)-4-amino-1¹-ethyl-2⁵-hydroxy-1²-(4-(methoxymethyl)pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (395.0 mg, 0.631 mmol). The reaction mixture was warmed to room temperature and stirred for 20 h. The mixture was then concentrated under reduced pressure, taken up in EtOAc (100 mL), and washed with brine (3×5 mL). The organic layer was dried over Na₂SO₄, filtered, and concentrated under reduced pressure. The residue was purified by normal phase chromatography to afford a crude solid (0.81 g), which was then purified by reversed phase chromatography (MeCN/H₂O) to afford the desired product (174 mg, 29% yield) as a solid. LCMS (ESI) m/z: [M+H] calcd for C₅₅H₆₇N₇O₉: 970.51; found 970.8.

### Step 6: Synthesis of (3S)—N-((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

To a solution of benzyl (3S)-3-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) pyrrolidine-1-carboxylate (174.0 mg, 0.179 mmol) in MeOH (20.0 mL) was added Pd/C (87.0 mg, 0.08 mmol) followed by 2% aq. HCl (one drop). The reaction mixture was stirred at room temperature under a H₂ atmosphere (1 atm) for 14 h, at which point the reaction mixture was purged with N₂, filtered, and concentrated under reduced pressure to afford the crude product (130 mg, 86.7% yield) as a solid. LCMS (ESI) m/z: [M+H] calcd for C₄₇H₆₁N₇O₇: 836.47; found 836.5.

### Intermediate 13. Synthesis of (2S)-2-(3-amino-N-methylpropanamido)-N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

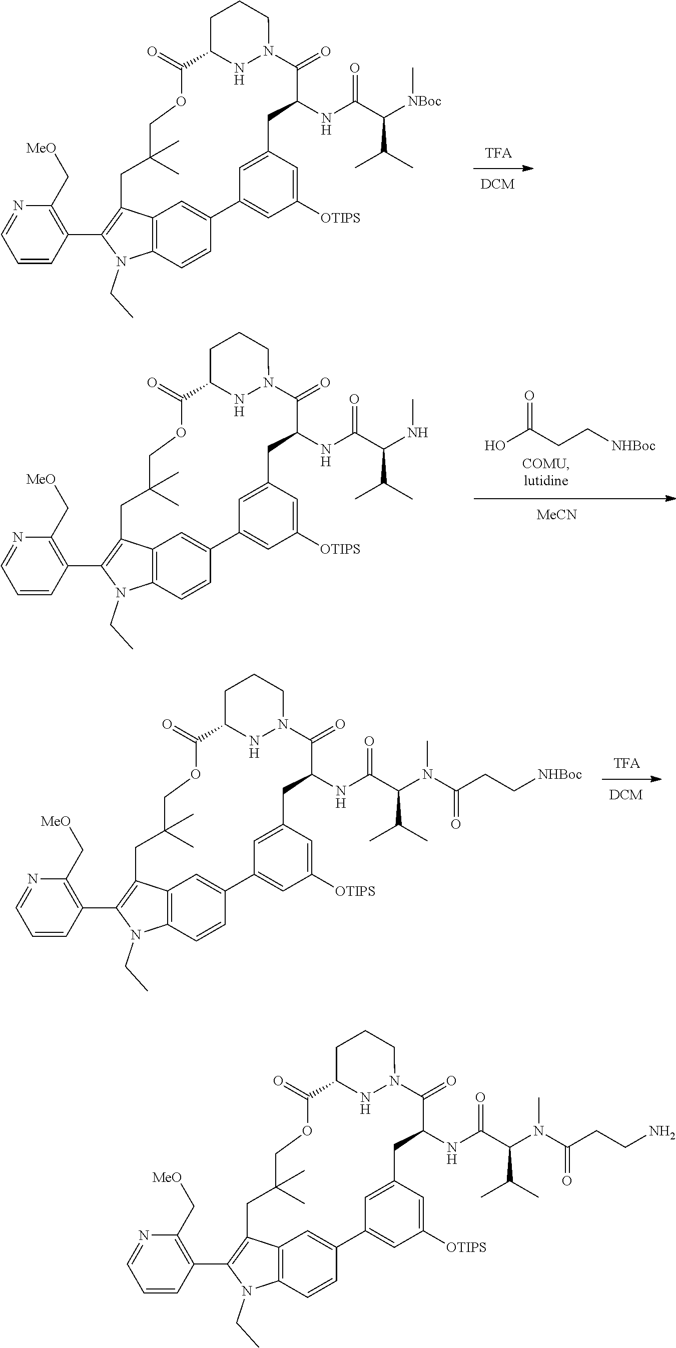

### Step 1: Synthesis of (2S)—N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide

To a solution of tert-butyl ((2S)-1-(((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (212.4 mg, 212 μmol) in DCM (500 μL) at 0° C. was added TFA (500 μL, 6.52 mmol). After 2 h, the reaction was diluted with DCM (10 mL) and H₂O (10 mL), and then sat. aq. NaHCO₃ was added until the solution was pH 9. The aqueous layer was extracted with DCM (10 mL) and the combined organic layers were dried over Na₂SO₄, filtered, and concentrated under reduced pressure to afford the crude product (194 mg, 103% yield). LCMS (ESI) m/z: [M+H] calcd for C₅₁H₇₄N₆O₆Si: 895.55; found 895.7.

### Step 2: Synthesis of tert-butyl (3-(((2S)-1-(((6³S,4S)-1¹-ethyl-1²-(2-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-3-oxopropyl) carbamate

To a mixture of (2S)—N-((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (150 mg, 167 μmol), COMU (88.5 mg, 206 μmol), and 3-((tert-butoxycarbonyl) amino) propanoic acid (39.6 mg, 209 μmol) in MeCN (1.66 mL) was added 2,6-lutidine (77.7 μL, 668 μmol). The reaction was stirred for 18 h at room temperature and then for 1 h at 55° C. The reaction mixture was cooled to room temperature and was concentrated under reduced pressure. The crude residue was purified by reverse phase chromatography (20→60% MeCN/H₂O) to afford the product (132 mg, 67% yield). LCMS (ESI) m/z: [M+H] calcd for C₅₉H₈₇N₇O₉Si: 1066.64; found 1066.7.

### Step 3: Synthesis of (2S)-2-(3-amino-N-methylpropanamido)-N-((6³S,4S)-1¹-ethyl-1²-(2-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of tert-butyl (3-(((2S)-1-(((6³S,4S)-1¹-ethyl-1²-(2-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-3-oxopropyl) carbamate (120 mg, 112 μmol) in DCM (560 μL) at 0° C. was added TFA (560 UL, 7.30 mmol). After 40 min, the reaction was diluted with DCM (10 mL) and then sat. aq. NaHCO₃ was added. The organic layer was dried over Na₂SO₄, filtered, and then concentrated under reduced pressure to afford the product (106 mg, 98% yield), which was used in the next step without purification. LCMS (ESI) m/z: [M+H] calcd for C₅₄H₇₉N₇O₇Si: 966.59; found 966.8.

### Intermediate 14. Synthesis of (2S)-2-cyclopentyl-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(methylamino) acetamide

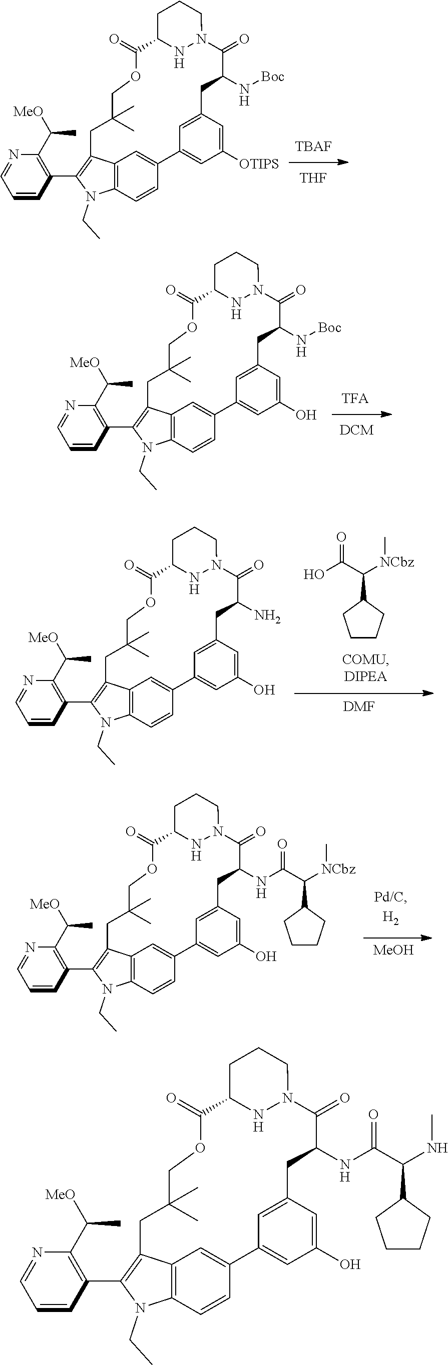

### Step 1: Synthesis of tert-butyl ((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred solution of tert-butyl ((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (18.0 g, 20.1 mmol) in THF (180 mL) at 0° C. was added a 1M solution of TBAF in THF (24.1 mL, 24.1 mmol). The mixture was stirred at 0° C. for 1 h, then diluted with brine (1.5 L), and extracted with EtOAc (3×1 L). The combined organic layers were washed with brine (2×500 mL), dried over anhydrous Na₂SO₄, filtered, and concentrated under reduced pressure. Purification by normal phase chromatography afforded the desired product (11.5 g, 69% yield). LCMS (ESI) m/z: [M+H] calcd for C₄₂H₅₃N₅O₇: 740.40; found 740.4.

### Step 2: Synthesis of (6³S,4S)-4-amino-1¹-ethyl-25-hydroxy-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a stirred solution of tert-butyl ((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (11.5 g, 15.5 mmol) in DCM (120 mL) at 0° C. was added TFA (60 mL, 808 mmol). The mixture was stirred at 0° C. for 1 h, then concentrated under reduced pressure and the residue again concentrated under reduced pressure with toluene (3×20 mL) to afford the desired crude product (12 g). LCMS (ESI) m/z: [M+H] calcd for C₃₇H₄₅N₅O₇: 640.35; found 640.6.

### Step 3: Synthesis of benzyl ((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) carbamate

To a stirred solution of (6³S,4S)-4-amino-1¹-ethyl-25-hydroxy-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (400.0 mg, 0.63 mmol) in DMF (4.0 mL) at 0° C. was added DIPEA (1.09 mL, 6.25 mmol) and(S)-2-(((benzyloxy) carbonyl) (methyl) amino)-2-cyclopentylacetic acid (255.0 mg, 0.88 mmol) followed by COMU (347.8 mg, 0.81 mmol). The resulting mixture was stirred at 0° C. for 1 h and was then diluted with H₂O (40 mL). The aqueous layer was extracted with EtOAc (3×15 mL) and the combined organic layers were washed with brine (2×10 mL), dried over Na₂SO₄, filtered, and concentrated under reduced pressure. The residue was purified by prep-TLC(25% EtOAc/pet. ether) to afford the desired product (510 mg, 80% yield). LCMS (ESI) m/z: [M+H] calcd for C₅₃H₆₄N₆O₈: 913.49; found 913.6.

### Step 4: Synthesis of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(methylamino) acetamide

To a stirred solution of benzyl ((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) carbamate (480.0 mg, 0.53 mmol), in MeOH (25 mL) was added Pd/C (200.0 mg, 1.88 mmol). The resulting mixture was placed under an atmosphere of H₂ (1 atm) and stirred for 2 h. The mixture was filtered, the filter cake was washed with MeOH (3×10 mL), and the filtrate was concentrated under reduced pressure to afford the desired crude product (440 mg). LCMS (ESI) m/z: [M+H] calcd for C₄₅H₅₈N₆O₆: 779.45; found 779.4.

### Intermediate 15. Synthesis of (2S)—N-((6³S,4S)-1¹-ethyl-25-hydroxy-1²-(4-(methoxymethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-3-(methylamino) propanamido) butanamide

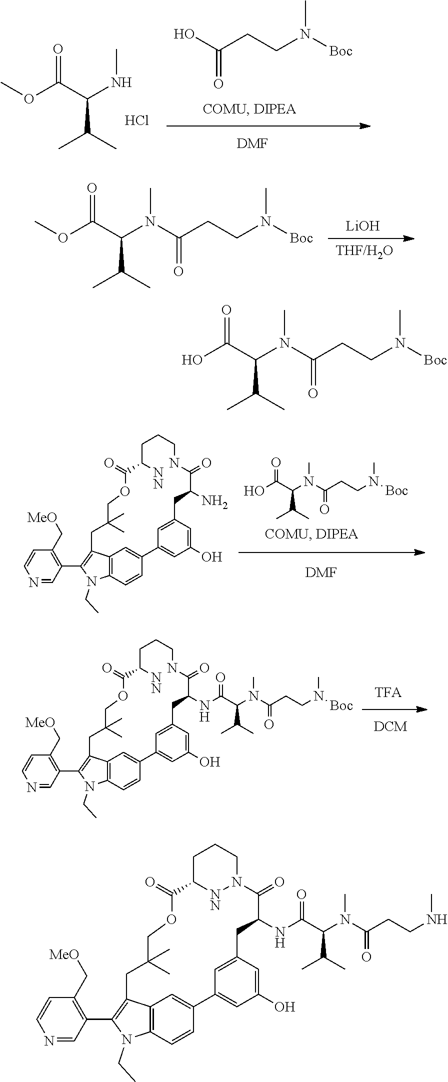

### Step 1: Synthesis of methyl N-(3-((tert-butoxycarbonyl) (methyl) amino) propanoyl)-N-methyl-L-valinate

To a solution of methyl methyl-L-valinate hydrochloride (1.0 g, 6.89 mmol) in DMF (20.0 mL) at 0° C. was added DIPEA (5.92 mL, 0.034 mmol), 3-((tert-butoxycarbonyl) (methyl) amino) propanoic acid (2.10 g, 0.010 mmol), and COMU (3.54 g, 8.27 mmol). The resulting mixture was stirred for 30 min and then quenched with H₂O (20 mL). The aqueous layer was extracted with EtOAc (3×20 mL) and the combined organic layers were washed with brine (3×20 mL), dried over Na₂SO₄, filtered, and concentrated under reduced pressure. The residue was purified by reverse phase chromatography (0→100% MeCN/H₂O) to afford the desired product (2 g, 87.9% yield). LCMS (ESI) m/z: [M+H] calcd for C₁₆H₃₀N₂O₅: 331.22; found 331.2.

### Step 2: Synthesis of N-(3-((tert-butoxycarbonyl) (methyl) amino) propanoyl)-N-methyl-L-valine

To a solution of N-(3-((tert-butoxycarbonyl) (methyl) amino) propanoyl)-N-methyl-L-valinate (1.0 g, 3.03 mmol) in THF (20.0 mL) and H₂O (4.0 mL) was added LiOH (0.14 g, 6.05 mmol). The resulting mixture was stirred for 3 h at room temperature. The mixture was acidified to pH 3 with HCl (1N) and was then extracted with EtOAc (3×20 mL). The combined organic layers were washed with brine (3×20 mL), dried over Na₂SO₄, filtered, and concentrated under reduced pressure to afford the desired crude product (800 mg, 83.6% yield). LCMS (ESI) m/z: [M+H] calcd for C₁₅H₂₈N₂O₅: 317.21; found 317.2.

### Step 3: Synthesis of tert-butyl (3-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-3-oxopropyl) (methyl) carbamate

To a solution of (6³S,4S)-4-amino-1¹-ethyl-25-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (600.0 mg, 0.96 mmol) in DMF (6.0 mL) at 0° C. was added DIPEA (1.67 mL, 9.59 mmol), N-(3-((tert-butoxycarbonyl) (methyl) amino) propanoyl)-N-methyl-L-valine (455.1 mg, 1.44 mmol), and COMU (492.5 mg, 1.15 mmol). The resulting mixture was stirred for 30 min and was then quenched with H₂O (60 mL). The aqueous layer was extracted with EtOAc (3×60 mL) and the combined organic layers were washed with brine (3×60 mL), dried over Na₂SO₄, filtered, and concentrated under reduced pressure. The residue was purified by reverse phase chromatography (0→100% MeCN/H₂O) to afford the desired product (650 mg, 73.4% yield). LCMS (ESI) m/z: [M+H] calcd for C₅₁H₆₉N₇O₉: 924.52; found 924.6.

### Step 4: Synthesis of (2S)—N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-3-(methylamino) propanamido) butanamide

To a solution of tert-butyl (3-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-3-oxopropyl) (methyl) carbamate (650.0 mg) in DCM (7.0 mL) at 0° C. was added TFA (3.5 mL). The resulting mixture was stirred for 30 min and was then concentrated under reduced pressure. The resulting residue was diluted with toluene (3×10 mL) and concentrated under reduced pressure to afford the desired crude product. LCMS (ESI) m/z: [M+H] calcd for C₄₆H₆₁N₇O₇: 824.47; found 824.6.

### Intermediate 16. Synthesis of (2S)-2-cyclopentyl-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(N-methyl-2-(methylamino) acetamido) acetamide

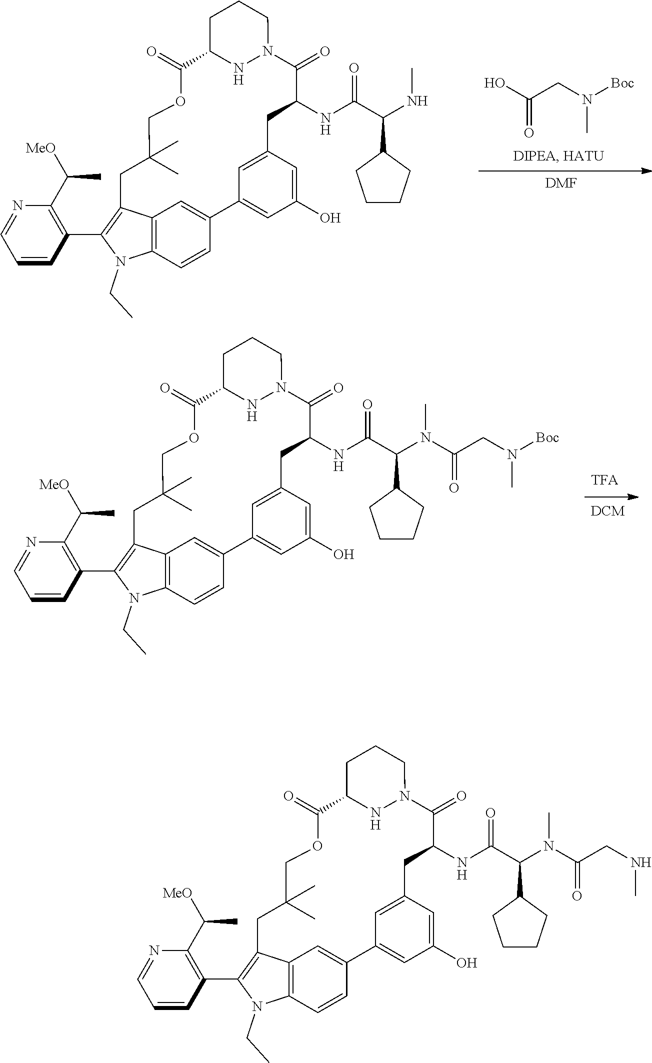

### Step 1: Synthesis of tert-butyl (2-(((1S)-1-cyclopentyl-2-(((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl) (methyl) carbamate

To a mixture of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(methylamino) acetamide (300.0 mg, 0.385 mmol), DIPEA (0.657 mL, 3.851 mmol), and N-(tert-butoxycarbonyl)-N-methylglycine (109.30 mg, 0.578) in DMF (3.0 mL) at 0° C. was added HATU (175.72 mg, 0.462 mmol). The resulting mixture was stirred at 0° C. for 30 min and was then diluted with H₂O (30 mL). The resulting mixture was extracted with EtOAc (3×30 mL). The combined organic layers were washed with brine (3×30 mL), dried with Na₂SO₄, filtered, and concentrated under reduced pressure. Purification by prep-TLC(50% EtOAc/pet. ether) to afford the desired product (300 mg, 82.0% yield). LCMS (ESI) m/z: [M+H] calcd for C₅₃H₇₁N₇O₉: 950.54; found 950.4.

### Step 2: Synthesis of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(N-methyl-2-(methylamino) acetamido) acetamide

To a mixture of tert-butyl (2-(((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl) (methyl) carbamate (300.0 mg, 0.316 mmol) in DCM (3.0 mL) at 0° C. was added TFA (1.50 mL). The resulting mixture was stirred at 0° C. for 30 min and was then concentrated under reduced pressure to afford the desired crude product. LCMS (ESI) m/z: [M+H] calcd for C₄₈H₆₃N₇O₇: 850.49; found 850.5.

### Intermediate 17. Synthesis of (2R,5R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,5-dimethylpyrrolidine-2-carboxamide

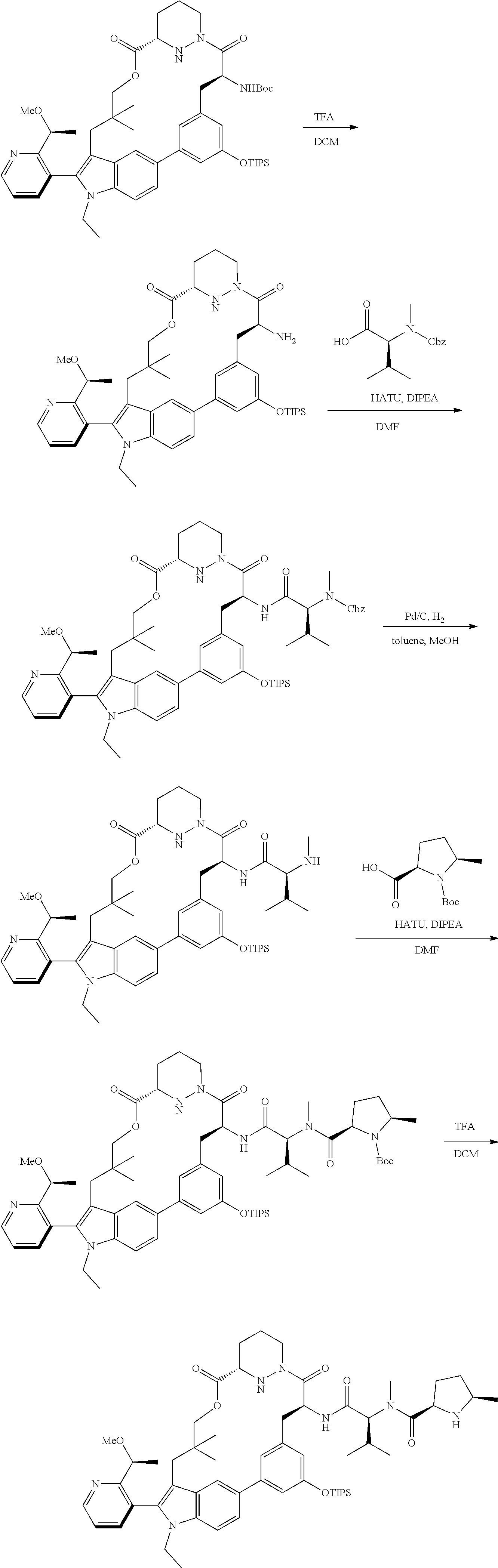

### Step 1: Synthesis of (6³S,4S)-4-amino-1¹-ethyl-1²-(2-((S)-1-methoxyethyl) pyridin-3-yl)-10,10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a solution of tert-butyl ((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (20.0 g, 22.315 mmol) in DCM (150.0 mL) at 0° C. was added TFA (50.0 mL). The resulting mixture was warmed to room temperature and stirred for 2 h and then concentrated under reduced pressure. The residue was dissolved in EtOAc (100 mL) and the solution was neutralized to pH 8 with sat. aq. NaHCO₃. The solution was extracted with EtOAc (3×150 mL) and the combined organic layers were washed with brine (100 mL), dried over Na₂SO₄, filtered, and concentrated under reduced pressure to afford the desired product (17.86 g, crude). LCMS (ESI) m/z: [M+H] calcd for C₄₆H₆₅N₅O₅Si: 796.49; found 795.5 .

### Step 2: Synthesis of benzyl ((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate

To a solution of (6³S,4S)-4-amino-1¹-ethyl-1²-(2-((S)-1-methoxyethyl) pyridin-3-yl)-10,10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (17.86 g, 22.433 mmol) and (2S)-2-[[(benzyloxy) carbonyl](methyl) amino]-3-methylbutanoic acid (8.93 g, 33.65 mmol) in DMF (150.0 mL) at 0° C. was added DIPEA (19.5 mL, 112.17 mmol) and HATU (17.06 g, 44.87 mmol). The resulting mixture was warmed to room temperature and stirred for 2 h. The reaction mixture was cooled to 0° C. and was quenched by the addition of H₂O (500 mL). The mixture was extracted with EtOAc (3×150 mL) and the combined organic layers were washed with brine (200 mL), dried over Na₂SO₄, filtered, and concentrated under reduced pressure. The residue was purified by normal phase chromatography (25% EtOAc/pet. ether) to afford the desired product (19.0 g, 81.2% yield). LCMS (ESI) m/z: [M+H] calcd for C₆₀H₈₂N₆O₈Si: 1043.61; found 1042.6 .

### Step 3: Synthesis of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide

To a solution of benzyl ((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (1.20 g, 1.150 mmol) in MeOH (1.2 mL) and toluene (1.2 mL) was added Pd/C (10%, 240 mg). The resulting mixture was placed under an atmosphere of H₂ (1 atm) and stirred overnight. The mixture was filtered and concentrated under reduced pressure to afford the desired product (1.05 g, 97.4% yield). LCMS (ESI) m/z: [M+H] calcd for C₅₂H₇₆N₆O₆Si: 909.57; found 909.3.

### Step 4: Synthesis of tert-butyl (2R,5R)-2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl)-5-methylpyrrolidine-1-carboxylate

To a solution of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (500 mg, 0.550 mmol) in DMF (5 mL) at 0° C. was added DIPEA (0.94 mL, 5.499 mmol) and (2R,5R)-1-(tert-butoxycarbonyl)-5-methylpyrrolidine-2-carboxylic acid (504.29 mg, 2.199 mmol) followed by HATU (627.23 mg, 1.650 mmol) in portions. The resulting mixture was warmed to room temperature and stirred for 1 h. Purification by reverse phase chromatography (0→100% MeCN/H₂O) afforded the desired product (147 mg, 22.2% yield). LCMS (ESI) m/z: [M+H] calcd for C₆₃H₉₃N₇O₉Si: 1120.69; found 1120.6.

### Step 5: Synthesis of (2R,5R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,5-dimethylpyrrolidine-2-carboxamide

To a solution of tert-butyl (2R,5R)-2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl)-5-methylpyrrolidine-1-carboxylate (150.0 mg, 0.134 mmol) in DCM at 0° C. was added TFA (1.50 mL, 13.155 mmol) dropwise. The resulting mixture was warmed to room temperature and stirred for 2 h and was then basified to pH 8 with sat. NaHCO₃. The resulting mixture was extracted with EtOAc (3×5 mL) and the combined organic layers were washed with brine (2×5 mL), dried with Na₂SO₄, filtered, and concentrated under reduced pressure to afford the desired product (85 mg, 54.1% yield). LCMS (ESI) m/z: [M+H] calcd for C₅₈H₈₅N₇O₇Si: 1020.64; found 1020.4.

### Intermediate 18. Synthesis of (2R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl) oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-1-methylpyrrolidine-2-carboxamide

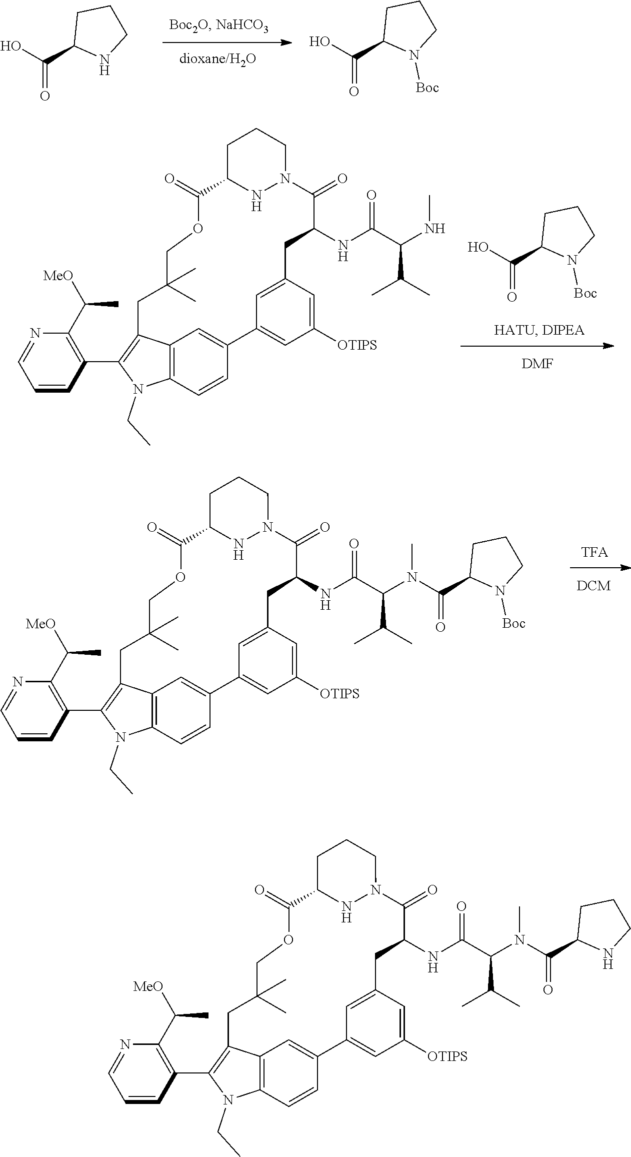

### Step 1: Synthesis of (tert-butoxycarbonyl)-D-proline

To a solution of D-proline (5.0 g, 43.43 mmol) in 1,4-dioxane (50 mL) and sat. NaHCO₃ (50 mL) at 0° C. was added Boc₂O (14.217 g, 65.143 mmol) in portions. The resulting mixture was stirred for 2 h at room temperature and was then extracted with EtOAc (100 mL). The aqueous layer was acidified to pH 6 with HCl and was then extracted into EtOAc (3×100 mL). The combined organic layers were washed with H₂O (2×100 mL), dried with Na₂SO₄, filtered, and concentrated under reduced pressure to afford the desired product which was used without further purification. LCMS (ESI) m/z: [M−H] calcd for C₁₀H₁₇NO₄: 214.11; found 214.0.

### Step 2: Synthesis of tert-butyl (2R)-2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) pyrrolidine-1-carboxylate

To a solution of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (142.03 mg, 0.660 mmol) in DMF was added DIPEA (0.710 mL, 5.499 mmol) followed by HATU (250.89 mg, 0.660 mmol) in portions. The resulting mixture was heated to 40° C. and stirred for 2 h. Purification by reverse phase chromatography (0→100% MeCN/H₂O) afforded the desired product (350 mg, 54.6% yield). LCMS (ESI) m/z: [M+H] calcd for C₆₂H₉₁N₇O₉Si: 1106.67; found 1106.8.

### Step 3: Synthesis of (2R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl) oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-1-methylpyrrolidine-2-carboxamide

To a solution of tert-butyl (2R)-2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) pyrrolidine-1-carboxylate (350.0 mg, 0.325 mmol) in DCM (4 mL) at 0° C. was added TFA (2.0 mL). The resulting mixture was stirred for 30 min at 0° C. and then was concentrated under reduced pressure. The residue was dissolved in toluene (5 mL) then concentrated under reduced pressure three times to afford the desired product which was used without further purification. LCMS (ESI) m/z: [M+H] calcd for C₅₇H₈₃N₇O₇Si: 1006.62; found 1006.4.

### Intermediate 19. Synthesis of (2R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylazetidine-2-carboxamide

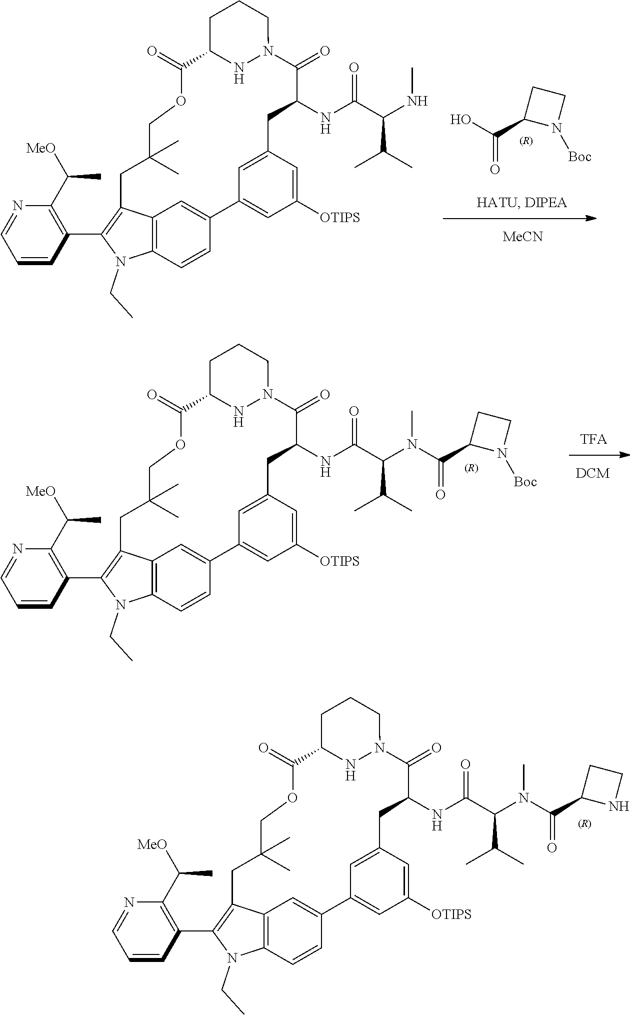

### Step 1: Synthesis of tert-butyl (2R)-2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) azetidine-1-carboxylate

To a mixture of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (1.0 g, 1.10 mmol), (R)-1-(tert-butoxycarbonyl) azetidine-2-carboxylic acid (0.33 g, 1.650 mmol) and HATU (1.25 g, 3.299 mmol) in MeCN (20 mL) at 0° C. was added DIPEA (0.94 mL, 5.499 mmol). The resulting mixture was stirred at 0° C. for 3 h and then was concentrated under reduced pressure. Purification by prep-TLC(10% MeOH/DCM) afforded the desired product (800 mg, 59.9% yield). LCMS (ESI) m/z: [M+H] calcd for C₆₁H₈₉N₇O₉Si: 1092.65; found 1092.6.

### Step 2: Synthesis of (2R)—N-((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylazetidine-2-carboxamide

To a mixture of tert-butyl (2R)-2-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) azetidine-1-carboxylate (400.0 mg, 0.366 mmol) in DCM (8.0 mL) at 0° C. was added TFA (4.0 mL). When the reaction was complete the mixture was concentrated under reduced pressure to afford the desired product (400 mg, crude). LCMS (ESI) m/z: [M+H] calcd for C₅₆H₈₁N₇O₇Si: 992.61; found 992.4.

### Intermediate 20. Synthesis of N-(sec-butyl)-5-((6³S,4S)-1¹-ethyl-25-hydroxy-10,10-dimethyl-4-((S)-3-methyl-2-(N-methyl-2-(methylamino) acetamido) butanamido)-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-methylnicotinamide

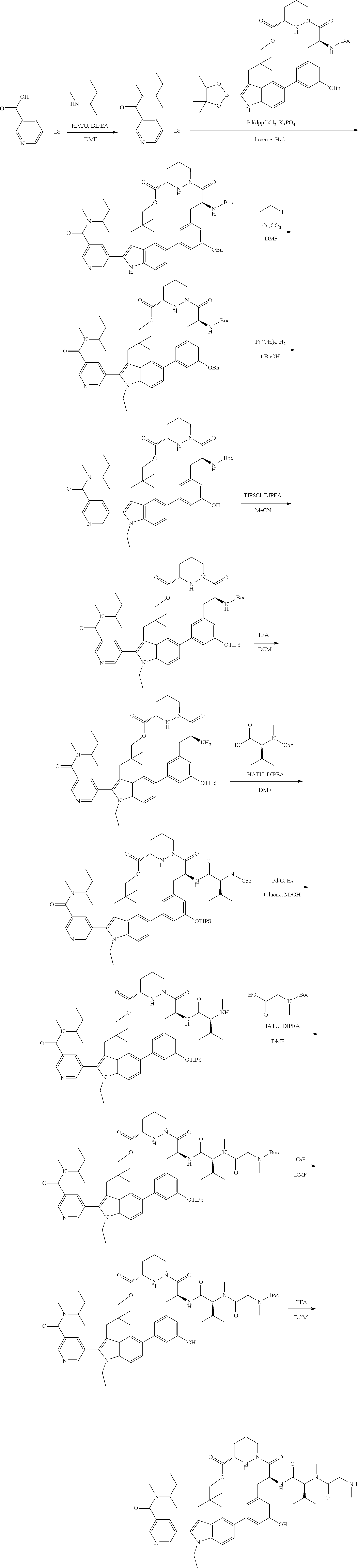

### Step 1: Synthesis of 5-bromo-N-(sec-butyl)-N-methylnicotinamide

To a solution of 5-bromonicotinic acid (2.0 g, 9.901 mmol) and HATU (5.65 g, 14.851 mmol) in DMF (40 mL) at 0° C. was added DIPEA (5.2 mL 9.9 mmol). The resulting mixture was stirred for 30 min at 0° C. and then N-methylbutan-2-amine (0.91 g, 10.396 mmol) was added. The resulting mixture was warmed to room temperature and stirred overnight, then diluted with H₂O (40 mL). The mixture was extracted with EtOAc (3×30 mL) and the combined organic layers were washed with brine (50 mL), dried over Na₂SO₄, filtered, and concentrated under reduced pressure. The residue was purified by silica gel chromatography (50% EtOAc/pet. ether) to afford the desired product (1.96 g, 73.2% yield). LCMS (ESI) m/z: [M+H] calcd for C₁₁H₁₅BrN₂O: 271.04; found 271.1.

### Step 2: Synthesis of tert-butyl ((6³S,4S)-2⁵-(benzyloxy)-1²-(5-(sec-butyl (methyl) carbamoyl) pyridin-3-yl)-10, 10-dimethyl-5,7-dioxo-6¹, 6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of 5-bromo-N-(sec-butyl)-N-methylnicotinamide (800.0 mg, 2.95 mmol) and K₃PO₃ (1.565 g, 7.376 mmol) in 1,4-dioxane (30.0 mL) and H₂O (6.0 mL) was added tert-butyl ((6³S,4S)-2⁵-(benzyloxy)-10, 10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (2.81 g, 3.540 mmol) and Pd(dppf)Cl₂ (215.87 mg, 0.295 mmol). The resulting mixture was heated to 85° C. and stirred for 3 h. The mixture was then cooled to room temperature, quenched with H₂O, and extracted with EtOAc (3×100 mL). The combined organic layers were washed with H₂O (100 mL), dried over Na₂SO₄, filtered, and concentrated. The residue was purified by silica gel chromatography (10% MeOH/DCM) to afford the desired product (2.2 g, crude). LCMS (ESI) m/z: [M+H] calcd for C₅₀H₆₀N₆O₇: 857.46; found 857.5.

### Step 3: Synthesis of tert-butyl ((6³S,4S)-25-(benzyloxy)-12-(5-(sec-butyl (methyl) carbamoyl) pyridin-3-yl)-1¹-ethyl-10, 10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate

To a solution of tert-butyl ((6³S,4S)-25-(benzyloxy)-12-(5-(sec-butyl (methyl) carbamoyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (2.10 g, 2.450 mmol) and CS₂CO₃ (2.39 g, 7.351 mmol) in DMF (20.0 mL) was added ethyl iodide (0.57 g, 3.675 mmol). The resulting mixture was stirred for 3 h at room temperature and was then quenched with H₂O (200 mL). The resulting mixture was extracted with EtOAc (3×100 mL) and the combined organic layers were washed with brine (50 mL), dried over Na₂SO₄, filtered, and concentrated under reduced pressure. The residue was purified by silica gel chromatography (10% MeOH/DCM) to afford the desired product (800 mg, 36.9% yield). LCMS (ESI) m/z: [M+H] calcd for C₅₂H₆₄N₆O₇: 885.49; found 885.5.

### Step 4: Synthesis of tert-butyl ((6³S,4S)-12-(5-(sec-butyl (methyl) carbamoyl) pyridin-3-yl)-11-ethyl-25-hydroxy-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-25-(benzyloxy)-12-(5-(sec-butyl (methyl) carbamoyl) pyridin-3-yl)-1¹-ethyl-10, 10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) carbamate (770.0 mg, 0.870 mmol) in tert-BuOH (20.0 mL) was added Pd (OH) 2/C(24.42 mg, 0.174 mmol). The resulting suspension was stirred overnight at 50° C. under a hydrogen atmosphere (1 atm). The mixture was then cooled to room temperature, filtered and the filter cake was washed with MeOH (3×30 mL). The filtrate was concentrated under reduced pressure to afford the desired product (810 mg, crude). LCMS (ESI) m/z: [M+H] calcd for C₄₅H₅₈N₆O₇: 795.44; found 795.5.

### Step 5: Synthesis of tert-butyl ((6³S,4S)-12-(5-(sec-butyl (methyl) carbamoyl) pyridin-3-yl)-11-ethyl-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-12-(5-(sec-butyl (methyl) carbamoyl) pyridin-3-yl)-1¹-ethyl-25-hydroxy-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (800.0 mg, 1.0 mmol) and DIPEA (0.876 mL, 5.031 mmol) in MeCN (10.0 mL) was added chlorotris (propan-2-yl) silane (291.02 mg, 1.509 mmol). The resulting mixture was stirred for 3 h and was then quenched with H₂O. The resulting mixture was extracted with EtOAc (3×50 mL) and the combined organic layers were washed with H₂O (3×30 mL), dried over Na₂SO₄, filtered, and concentrated under reduced pressure. The residue was purified by silica gel chromatography (10% MeOH/DCM) to afford the desired product (800 mg, 83.6% yield). LCMS (ESI) m/z: [M+H] calcd for C₅₄H₇₈N₆O₇Si: 951.58; found 950.8.

### Step 6: Synthesis of 5-((6³S,4S)-4-amino-1¹-ethyl-10, 10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-(sec-butyl)-N-methylnicotinamide

To a solution of tert-butyl ((6³S,4S)-12-(5-(sec-butyl (methyl) carbamoyl) pyridin-3-yl)-1¹-ethyl-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (720.0 mg, 0.757 mmol) in DCM (10.0 mL) at 0° C. was added TFA (3.0 mL, 40.4 mmol). The resulting mixture was stirred for 2 h and was then concentrated under reduced pressure. The residue was cooled to at 0° C. and neutralized with sat. aq. NaHCO₃. The resulting mixture was extracted with EtOAc (3×100 mL) and the combined organic layers were washed with H₂O (3×30 mL), dried over Na₂SO₄, filtered, and concentrated under reduced pressure to afford the desired product (540 mg, crude). LCMS (ESI) m/z: [M+H] calcd for C₄₉H₇₀N₆O₅Si: 851.53; found 851.8.

### Step 7: Synthesis of benzyl ((2S)-1-(((6³S,4S)-12-(5-(sec-butyl (methyl) carbamoyl) pyridin-3-yl)-1¹-ethyl-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate

To a solution of 5-((6³S,4S)-4-amino-1¹-ethyl-10, 10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-(sec-butyl)-N-methylnicotinamide (530.0 mg, 0.623 mmol) and N-((benzyloxy) carbonyl)-N-methyl-L-valine (198.23 mg, 0.747 mmol) in DMF (10.0 mL) were added HATU (473.49 mg, 1.245 mmol) and DIPEA (0.542 mL, 3.113 mmol). The resulting mixture was stirred for 2 h and was then quenched with H₂O and extracted with EtOAc (3×50 mL). The combined organic layers were washed with H₂O (3×30 mL), dried over Na₂SO₄, filtered, and concentrated under reduced pressure. The residue was purified by silica gel chromatography (10% MeOH/DCM) to afford the desired product (720 mg, crude). LCMS (ESI) m/z: [M+H] calcd for C₆₃H₈₇NO₈Si: 1098.65; found 1098.7.

### Step 8: Synthesis of N-(sec-butyl)-5-((6³S,4S)-1¹-ethyl-10,10-dimethyl-4-((S)-3-methyl-2-(methylamino) butanamido)-5, 7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-methylnicotinamide

To a solution of benzyl ((2S)-1-(((6³S,4S)-12-(5-(sec-butyl (methyl) carbamoyl) pyridin-3-yl)-11-ethyl-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamate (670.0 mg, 0.610 mmol) in toluene (10.0 mL) and MeOH (1.0 mL) was added Pd/C (12.98 mg, 0.122 mmol). The suspension was stirred overnight under a hydrogen atmosphere (1 atm) and was then filtered, and the filter cake washed with MeOH (3×50 mL). The filtrate was concentrated under reduced pressure to afford the desired product (600 mg, crude). LCMS (ESI) m/z: [M+H] calcd for C₅₅H₈₁N₇O₆Si: 964.61; found 964.8.

### Step 9: Synthesis of tert-butyl (2-(((2S)-1-(((6³S,4S)-1²-(5-(sec-butyl (methyl) carbamoyl) pyridin-3-yl)-1¹-ethyl-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate

To a solution of N-(sec-butyl)-5-((6³S,4S)-1¹-ethyl-10, 10-dimethyl-4-((S)-3-methyl-2-(methylamino) butanamido)-5, 7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-methylnicotinamide (490.0 mg, 0.508 mmol) and N-(tert-butoxycarbonyl)-N-methylglycine (114.4 mg, 0.610 mmol) in DMF (10.0 mL) was added HATU (386.39 mg, 1.016 mmol) and DIPEA (0.443 mL, 2.540 mmol). The resulting mixture was stirred for 2 h and was then quenched with H₂O and extracted with EtOAc (3×30 mL). The combined organic layers were washed with H₂O (3×30 mL), dried over Na₂SO₄, filtered, and concentrated under reduced pressure to afford the desired product (560 mg, 79.3% yield). LCMS (ESI) m/z: [M+H] calcd for C₆₃H₉₄N₈O₉Si: 1135.70; found 1136.3.

### Step 10: Synthesis of tert-butyl (2-(((2S)-1-(((6³S,4S)-12-(5-(sec-butyl (methyl) carbamoyl) pyridin-3-yl)-1¹-ethyl-25-hydroxy-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate

To a solution of tert-butyl (2-(((2S)-1-(((6³S,4S)-12-(5-(sec-butyl (methyl) carbamoyl) pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate (540.0 mg, 0.476 mmol) in DMF (10.0 mL) was added CsF (288.94 mg, 1.90 mmol). The resulting mixture was stirred for 2 h and was then quenched with H₂O and extracted with EtOAc (3×50 mL). The combined organic layers were washed with H₂O (3×30 mL), dried over Na₂SO₄, filtered, and concentrated under reduced pressure. The residue was purified by silica gel chromatography (10% MeOH/DCM) to afford the desired product (430 mg, crude). LCMS (ESI) m/z: [M+H] calcd for C₅₄H₇₄N₈O₉: 979.57; found 980.0.

### Step 11: Synthesis of N-(sec-butyl)-5-((6³S,4S)-1¹-ethyl-25-hydroxy-10, 10-dimethyl-4-((S)-3-methyl-2-(N-methyl-2-(methylamino) acetamido) butanamido)-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-N-methylnicotinamide

To a solution of tert-butyl (2-(((2S)-1-(((6³S,4S)-1²-(5-(sec-butyl (methyl) carbamoyl) pyridin-3-yl)-1¹-ethyl-2⁵-hydroxy-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamate (400.0 mg, 0.408 mmol) in DCM (10.0 mL) at 0° C. was added TFA (3.0 mL, 40.4 mmol). The reaction was stirred for 1 h and was then quenched with sat. aq. NaHCO₃. The mixture was extracted with EtOAc (3×50 mL) and the combined organic layers were washed with H₂O (3×30 mL), dried over Na₂SO₄, filtered, and concentrated under reduced pressure to afford the desired product (380 mg, crude). LCMS (ESI) m/z: [M+H] calcd for C₄₉H₆₆N₈O₇: 879.51; found 879.5.

### Intermediate 21. Synthesis of (2S)-2-(2-amino-N-methylacetamido)-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

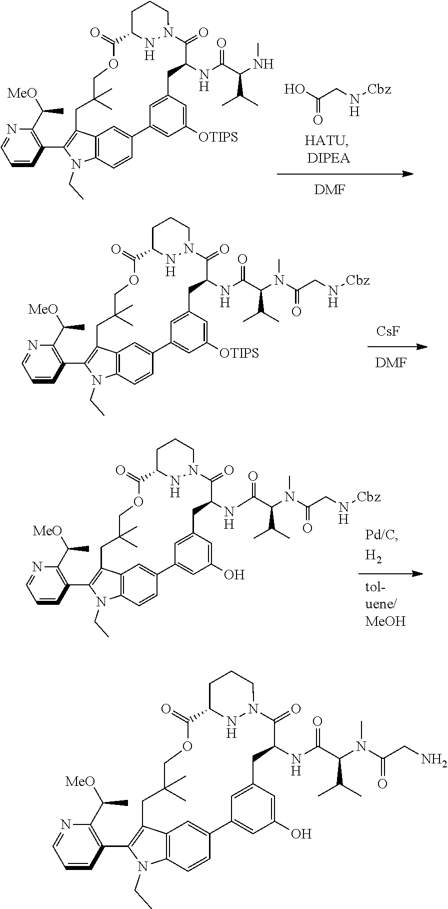

### Step 1: Synthesis of benzyl (2-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) carbamate

To a solution of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (2.50 g, 2.75 mmol) and ((benzyloxy) carbonyl) glycine (690 mg, 3.30 mmol) in DMF (25 mL) at 0° C. was added HATU (2.10 g, 5.50 mmol) followed by DIPEA (1.5 mL, 8.25 mmol). The reaction mixture was stirred for 2 h and was then quenched with H₂O and extracted with EtOAc (3×50 mL). The combined organic layers were washed with H₂O (3×10 mL), dried with Na₂SO₄, filtered, and concentrated under reduced pressure. Purification by silica gel column chromatography (50% EtOAC/hexanes) afforded desired product (2.0 g, 72% yield). LCMS (ESI) m/z: [M+H] calcd for C₆₂H₈₅N₇O₉Si: 1100.63; found 1100.7.

### Step 2: Synthesis of benzyl (2-(((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) carbamate

To a solution of benzyl (2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) carbamate (400 mg, 0.36 mmol) in DMF at 0° C. was added CsF (220 mg, 1.5 mmol). The reaction mixture was stirred for 2 h and was then quenched with H₂O and extracted with EtOAc (3×50 mL). The combined organic layers were washed with H₂O (3×10 mL), dried with Na₂SO₄, filtered, and concentrated under reduced pressure to afford the desired product (300 mg, 87% yield). LCMS (ESI) m/z: [M+H] calcd for C₅₃H₆₅N₇O₉: 944.49; found 944.4.

### Step 3: Synthesis of (2S)-2-(2-amino-N-methylacetamido)-N-((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of benzyl (2-(((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) carbamate (300 mg, 0.32 mmol) in toluene (10 mL) and MeOH (1 mL) was added Pd/C (50 mg, 0.47 mmol). The suspension was stirred overnight under an atmosphere of hydrogen (1 atm). The reaction mixture was then was filtered and the filter cake was washed with EtOAc (3×10 mL). The filtrate was concentrated under reduced pressure to afford the desired product (180 mg, 43% yield). LCMS (ESI) m/z: [M+H] calcd for C₄₅H₅₉N₇O₇: 810.46; found 810.5.

### Intermediate 22. (3S,4R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl) oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,4-dimethylpyrrolidine-3-carboxamide

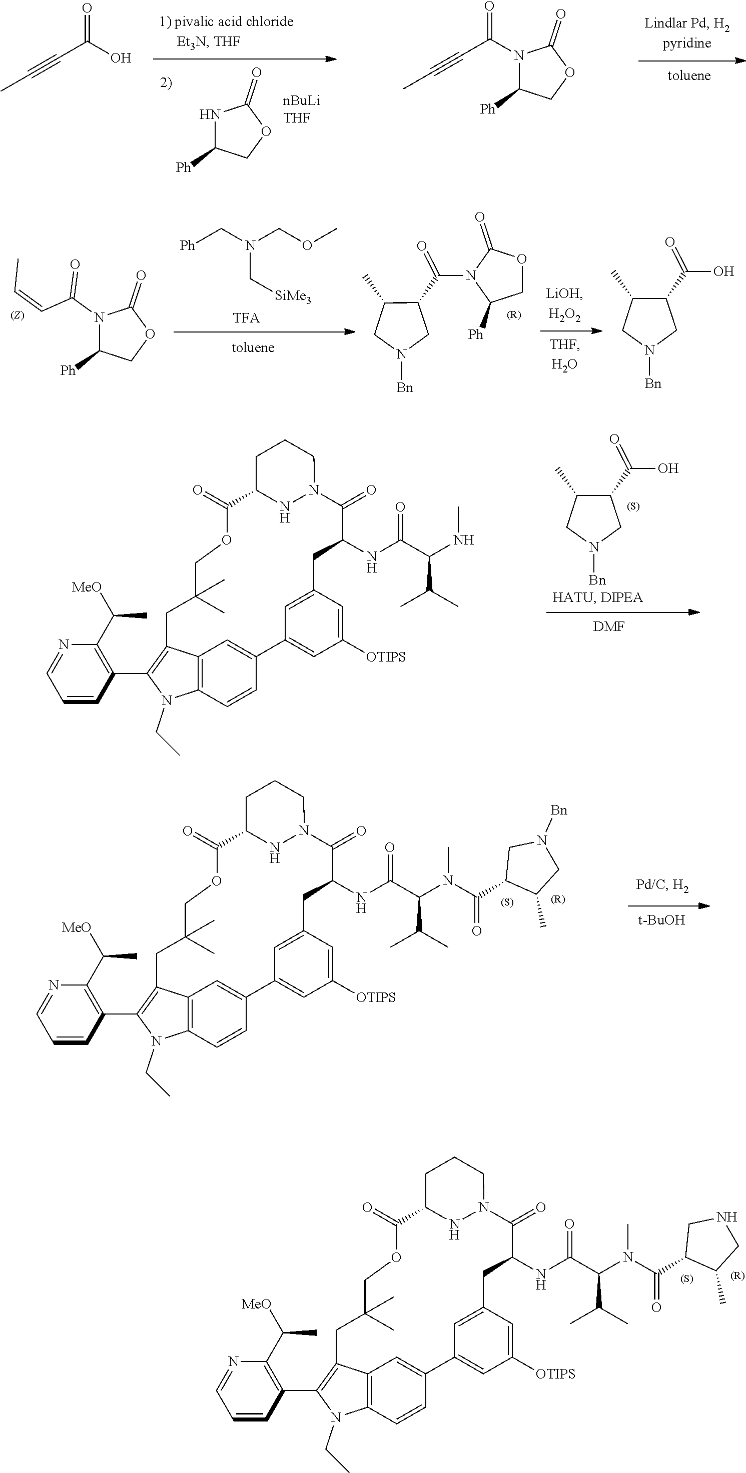

### Step 1: Synthesis of (R)-3-(but-2-ynoyl)-4-phenyloxazolidin-2-one

To a solution of 2-butynoic acid (5.0 g, 59.47 mmol) in THF (100 mL) at −78° C. was added pivalic acid chloride (7.39 g, 61.26 mmol) and Et₃N (6.2 mL, 61.85 mmol) and then the mixture was stirred for 15 min and then warmed to 0° C. and stirred for 45 min. In a second flask, to a solution of (4R)-4-phenyl-1,3-oxazolidin-2-one (9.70 g, 59.47 mmol) in THF (100 mL) at −78° C. was added n-BuLi (2.5 M in hexane, 25 mL, 62.5 mmol). The mixture was stirred at −78° C. for 15 min and was then added to the initial mixture. The combined solutions were warmed to room temperature and stirred overnight. The reaction solution was quenched with sat. NH₄Cl (200 mL) and then the mixture was extracted with EtOAc (3×100 mL). The combined organic layers were washed with brine (200 mL), dried over Na₂SO₄, filtered, and concentrated under reduced pressure. Purification by normal phase chromatography (20% EtOAc/pet. ether) afforded the desired product (6.0 g, 44.0% yield). LCMS (ESI) m/z: [M+H] calcd for C₁₃H₁₁NO₃: 230.08; found 229.9.

### Step 2: Synthesis of (R,Z)-3-(but-2-enoyl)-4-phenyloxazolidin-2-one

To a solution of (R)-3-(but-2-ynoyl)-4-phenyloxazolidin-2-one (6.0 g, 26.17 mmol) in pyridine (6.0 mL) and toluene (60.0 mL) at 0° C. was added Lindlar Pd catalyst (594.57 mg, 2.88 mmol). The resulting mixture was stirred for 30 min at 0° C. under a hydrogen atmosphere (1 atm). The mixture was filtered, and the filter cake was washed with toluene (10.0 mL). The filtrate was concentrated under reduced pressure to afford the desired product (5.5 g, crude). LCMS (ESI) m/z: [M+H] calcd for C₁₃H₁₃NO₃: 232.10; found 231.9.

### Step 3: (R)-3-((3S,4R)-1-benzyl-4-methylpyrrolidine-3-carbonyl)-4-phenyloxazolidin-2-one

To a solution of (R,Z)-3-(but-2-enoyl)-4-phenyloxazolidin-2-one (3.0 g, 12.97 mmol) and benzyl (methoxymethyl) [(trimethylsilyl)methyl]amine (3.70 g, 15.57 mmol) in toluene (20.0 mL) at 0° C. was added TFA (1.30 mL, 0.87 mmol). The resulting mixture was warmed to room temperature and stirred overnight. The mixture was then concentrated under reduced pressure. The residue was purified by silica gel column chromatography (20% EtOAc/pet. ether) to afford the desired product (2 g, 42.3% yield). LCMS (ESI) m/z: [M+H] calcd for C₂₂H₂₄N₂O₃: 365.19; found 365.2.

### Step 4: Synthesis of (3S,4R)-1-benzyl-4-methylpyrrolidine-3-carboxylic acid

A solution of LiOH·H₂O (0.16 g, 6.860 mmol) and H₂O₂ (0.13 g, 3.76 mmol) in H₂O (5 mL) was added to a solution of (R)-3-((3S,4R)-1-benzyl-4-methylpyrrolidine-3-carbonyl)-4-phenyloxazolidin-2-one (1.0 g, 2.74 mmol) in THF (15.0 mL) at 0° C. The resulting mixture was stirred for 2 h and was then quenched with H₂O (30 mL) and sodium sulfite (0.69 g, 5.48 mmol) and the solution was extracted with EtOAc (2×50 mL). The aqueous phase was adjusted to pH 4 with NaH₂PO₄·H₂O and 10% HCl, and the brine was added. The solution was extracted with i-PrOH/DCM (1:3, 5×50 mL) and the combined organic layers were washed with brine (40 mL), dried over Na₂SO₄, filtered, and concentrated under reduced pressure to afford the desired product (400 mg, crude). LCMS (ESI) m/z: [M+H] calcd for C₁₃H₁₇NO₂: 220.14; found 220.2.

### Step 5: Synthesis of (3S,4R)-1-benzyl-N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,4-dimethylpyrrolidine-3-carboxamide

To a mixture of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (414.67 mg, 0.456 mmol) and (3S,4R)-1-benzyl-4-methylpyrrolidine-3-carboxylic acid (200.0 mg, 0.912 mmol) in DMF (5.0 mL) at 0° C. was added HATU (693.58 mg, 1.824 mmol) and DIPEA (0.794 mL, 4.560 mmol). The resulting mixture was warmed to room temperature and stirred for 2 h. The reaction was quenched with the addition of sat. aq. NH₄Cl (40 mL) and then extracted with EtOAc (3×30 mL). The combined organic layers were washed with brine (2×20 mL), dried over Na₂SO₄, filtered, and concentrated under reduced pressure. The residue was purified by prep-TLC (9% MeOH/DCM) to afford the desired product (350 mg, 34.6% yield). LCMS (ESI) m/z: [M+H] calcd for C₆₅H₉₁N₇O₇Si: 1110.68; found 1110.9.

### Step 6: (3S,4R)—N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,4-dimethylpyrrolidine-3-carboxamide

To a solution of (3S,4R)-1-benzyl-N-((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N,4-dimethylpyrrolidine-3-carboxamide (300.0 mg, 0.270 mmol) in t-BuOH (10.0 mL) was added Pd/C (60.08 mg, 0.565 mmol). The resulting suspension was stirred overnight under a hydrogen atmosphere (1 atm). The mixture was then filtered, the filter cake was washed with MeOH (2×5 mL), and the filtrate was concentrated under reduced pressure to afford the desired product (280 mg, crude). LCMS (ESI) m/z: [M+H] calcd for C₅₈H₈₅N₇O₇Si: 1020.64; found 1020.8.

### Intermediate 23. Synthesis of (2S)—N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)—N-methyl-2-(methylamino) propanamido) butanamide

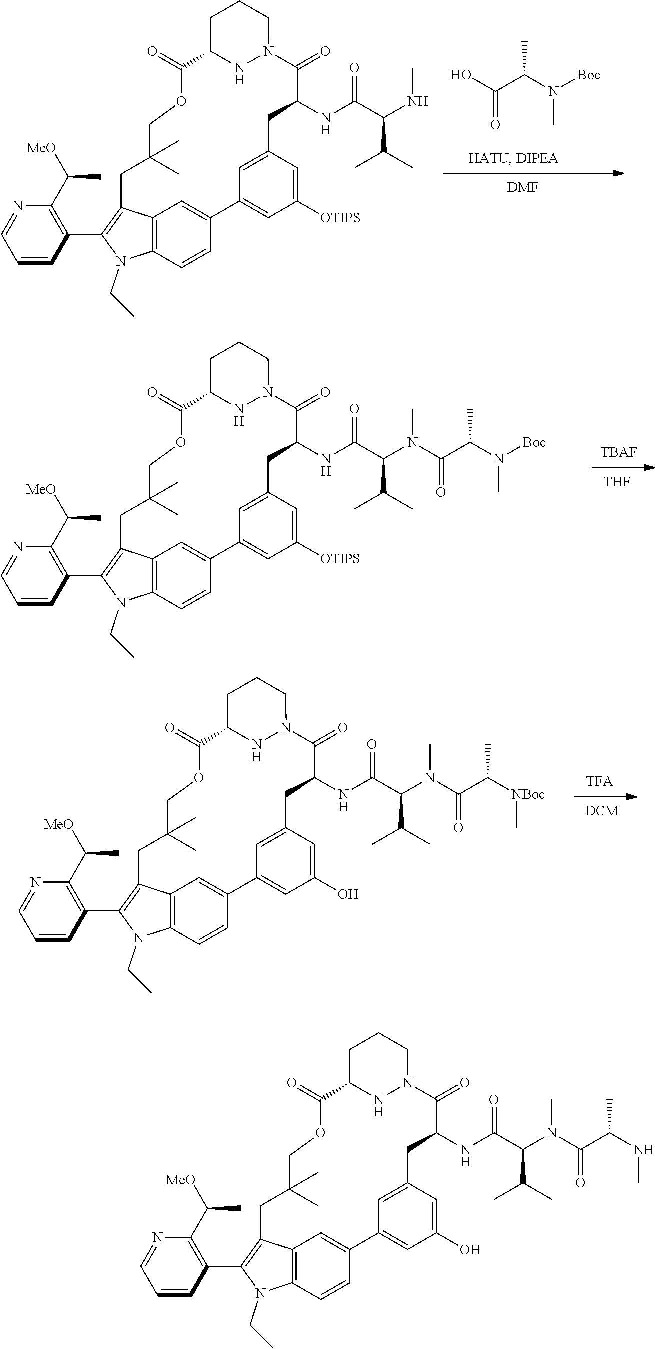

### Step 1: Synthesis of tert-butyl ((2S)-1-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-1-oxopropan-2-yl) (methyl) carbamate

To a solution of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (500.0 mg, 0.55 mmol), DIPEA (480 mL, 2.75 mmol) and (2S)-2-[(tert-butoxycarbonyl) (methyl) amino]propanoic acid (167.63 mg, 0.825 mmol) in DMF (5.0 mL) at 0° C. was added HATU (271.80 mg, 0.715 mmol). The mixture was warmed to room temperature and stirred for 4 h. The reaction was then quenched with H₂O and extracted with EtOAc (2×10 mL). The combined organic layers were washed with brine (5 mL), dried over Na₂SO₄, filtered, and concentrated under reduced pressure. The residue was purified by silica gel column chromatography (50% EtOAc/pet. ether) to afford the desired product (550 mg, 91.4% yield). LCMS (ESI) m/z: [M+H] calcd for C₆₁H₉₁N₇O₉Si: 1094.67; found 1094.5.

### Step 2: Synthesis of tert-butyl ((2S)-1-(((2S)-1-(((6³S,4S)-1¹-ethyl-25-hydroxy-1²-(2-((S)-1-methoxyethyl) pyridin-3-yl)-10, 10-dimethyl-5,7-dioxo-61, 62,63,64,65,66-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-1-oxopropan-2-yl) (methyl) carbamate

To a solution of tert-butyl ((2S)-1-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-1-oxopropan-2-yl) (methyl) carbamate (540 mg, 0.493 mmol) in THF (5.0 mL) at 0° C. was added TBAF (1M in THF, 0.59 mL, 0.592 mmol). The mixture was warmed to room temperature and stirred for 30 min. The reaction was quenched with H₂O and was then extracted with EtOAc (2×10 mL). The combined organic layers were washed with brine (10 mL), dried over Na₂SO₄, filtered, After filtration, and concentrated under reduced pressure. The residue was purified by silica gel column chromatography (20% EtOAc/pet. ether) to afford the desired product (320 mg, 69.1% yield). LCMS (ESI) m/z: [M+H] calcd for C₅₂H₇₁N₇O₉: 938.534; found 938.4.

### Step 3: Synthesis of (2S)—N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)—N-methyl-2-(methylamino) propanamido) butanamide

To a solution of tert-butyl ((2S)-1-(((2S)-1-(((6³S,4S)-1¹-ethyl-25-hydroxy-1²-(2-((S)-1-methoxyethyl) pyridin-3-yl)-10, 10-dimethyl-5,7-dioxo-61, 62,63,64,65,66-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-1-oxopropan-2-yl) (methyl) carbamate (300.0 mg, 0.320 mmol) in DCM (3.0 mL) at 0° C. and was added TFA (1.0 mL). The mixture was warmed to room temperature and stirred for 2 h. The mixture was concentrated under reduced pressure to afford the desired product (300 mg, crude). LCMS (ESI) m/z: [M+H] calcd for C₄₇H₆₃N₇O₇: 838.49; found 838.4.

### Intermediate 24. Synthesis of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

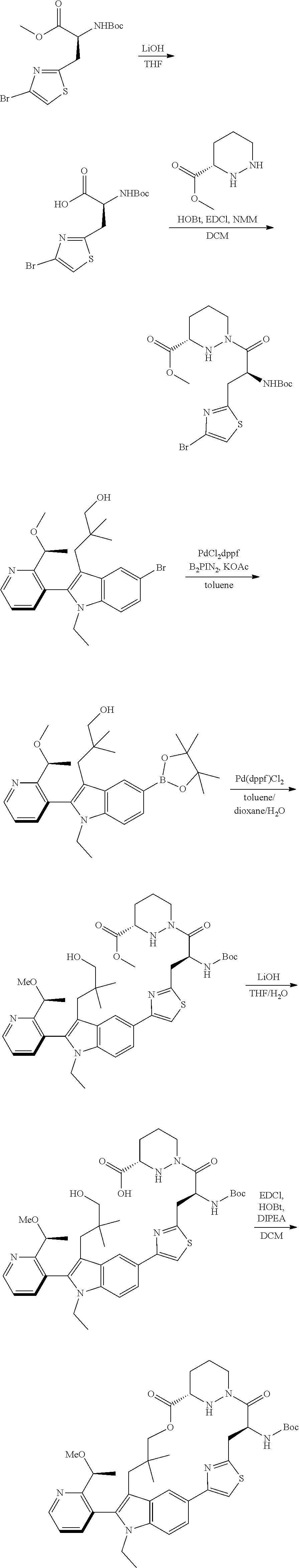

### Step 1: Synthesis of(S)-3-(4-bromothiazol-2-yl)-2-((tert-butoxycarbonyl) amino) propanoic acid

To a solution of methyl (2S)-3-(4-bromo-1,3-thiazol-2-yl)-2-[(tert-butoxycarbonyl) amino]propanoate (110 g, 301.2 mmol) in THF (500 mL) and H₂O (200 mL) at room temperature was added LiOH (21.64 g, 903.6 mmol). The resulting solution was stirred for 1 h and was then concentrated under reduced pressure. The resulting residue was adjusted to pH 6 with 1 M HCl and then extracted with DCM (3×500 mL). The combined organic layers were, dried over Na₂SO₄, filtered, and concentrated under reduced pressure to afford the desired product (108 g, crude). LCMS (ESI) m/z: [M+H] calcd for C₁₁H₁₅BrN₂O₄S: 351.00; found 351.0.

### Step 2: Synthesis of methyl(S)-1-((S)-3-(4-bromothiazol-2-yl)-2-((tert-butoxycarbonyl) amino) propanoyl) hexahydropyridazine-3-carboxylate

To a solution of(S)-3-(4-bromothiazol-2-yl)-2-((tert-butoxycarbonyl) amino) propanoic acid (70 g, 199.3 mmol) in DCM (500 mL) at 0° C. was added methyl (3S)-1,2-diazinane-3-carboxylate bis (trifluoroacetic acid) salt (111.28 g, 298.96 mmol), NMM (219.12 mL. 1993.0 mmol), EDCI (76.41 g, 398.6 mmol) and HOBt (5.39 g, 39.89 mmol). The resulting solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel chromatography (0→50% EtOAc/pet. ether) to afford the desired product (88.1 g, 92.6% yield). LCMS (ESI) m/z: [M+H] calcd for C₁₇H₂₅BrN₄O₅S: 477.08; found 477.1.

### Step 3: Synthesis of(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol

To a solution of 3-(5-bromo-1-ethyl-2-(2-((S)-1-methoxyethyl) pyridin-3-yl)-1H-indol-3-yl)-2,2-dimethylpropan-1-ol (60 g, 134.7 mmol) in toluene (500 mL) at room temperature was added bis (pinacolato)diboron (51.31 g, 202.1 mmol), Pd(dppf)Cl₂ (9.86 g, 13.48 mmol) and KOAc (26.44 g, 269.4 mmol). Then reaction mixture was then heated to 90° C. and stirred for 2 h. The reaction solution was then cooled to room temperature and concentrated under reduced pressure. Purification by silica gel chromatography (0→50% EtOAc/pet. ether) afforded the desired product (60.6 g, 94.0% yield). LCMS (ESI) m/z: [M+H] calcd for C₂₉H₄₁BN204: 493.32; found 493.3.

### Step 4: Synthesis of methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate

To a solution of(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl(S)-1-((S)-3-(4-bromothiazol-2-yl)-2-((tert-butoxycarbonyl) amino) propanoyl) hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The resulting mixture was extracted with EtOAc (3×1000 mL) and the combined organic layers were dried over Na₂SO₄, filtered, and concentrated under reduced pressure. The residue was purified by silica gel chromatography (0→90% EtOAc/pet. ether) to afford the desired product (39.7 g, 85.4% yield). LCMS (ESI) m/z: [M+H] calcd for C₄₀H₅₄N₆O₇S: 763.39; found 763.3.

### Step 5: Synthesis of(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid

To a solution of methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate (39.7 g, 52.0 mmol) in THF (400 mL) and H₂O (100 mL) at room temperature was added LiOH·H₂O (3.74 g, 156.2 mmol). The resulting mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to afford the desired product (37.9 g, crude). LCMS (ESI) m/z: [M+H] calcd for C₃₉H₅₂N₆O₇S: 749.37; found 749.4.

### Step 6: Synthesis of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate

To a solution of(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel chromatography (0→70% EtOAc/pet. ether) to afford the desired product (30 g, 81.1% yield). LCMS (ESI) m/z: [M+H] calcd for C₃₉H₅₀N₆O₆S: 731.36; found 731.3.

### Intermediate 25. Synthesis of (6³S,4S)-4-amino-1¹-ethyl-25-hydroxy-1²-(2-((S)-1-methoxyethyl)-5-(1-methylpiperidin-4-yl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

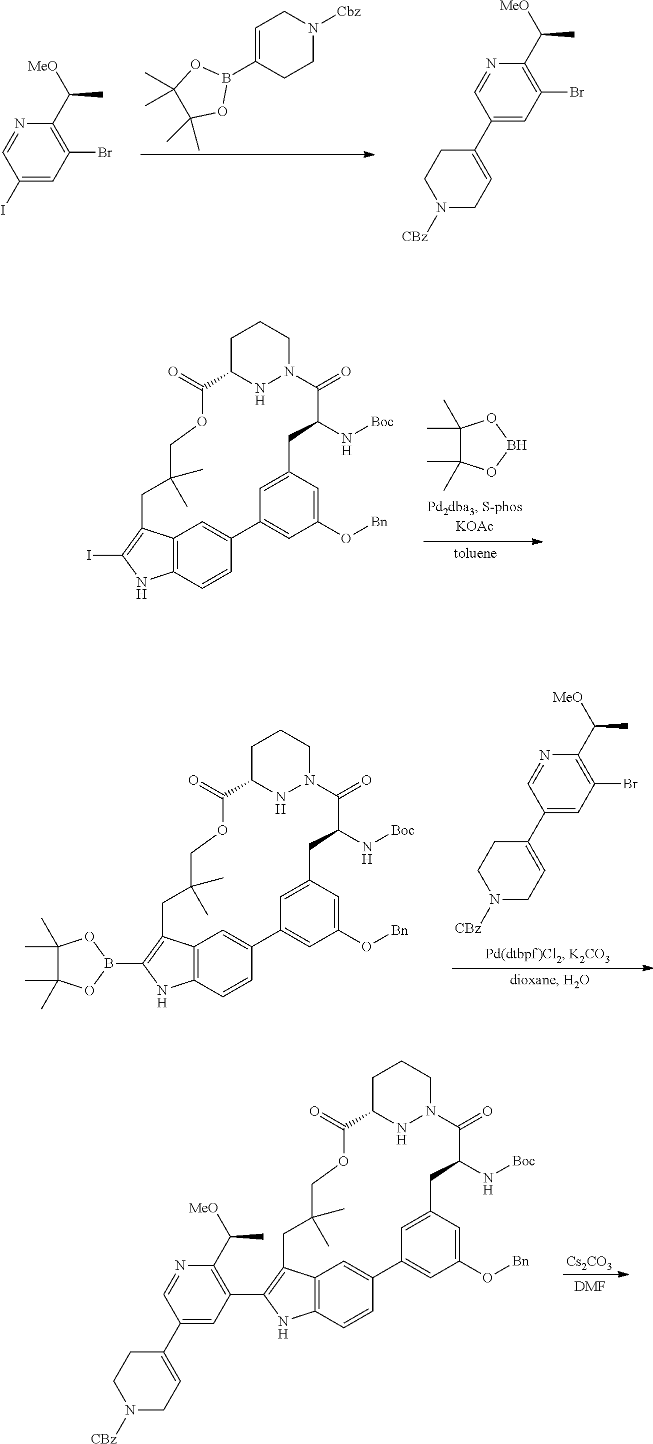

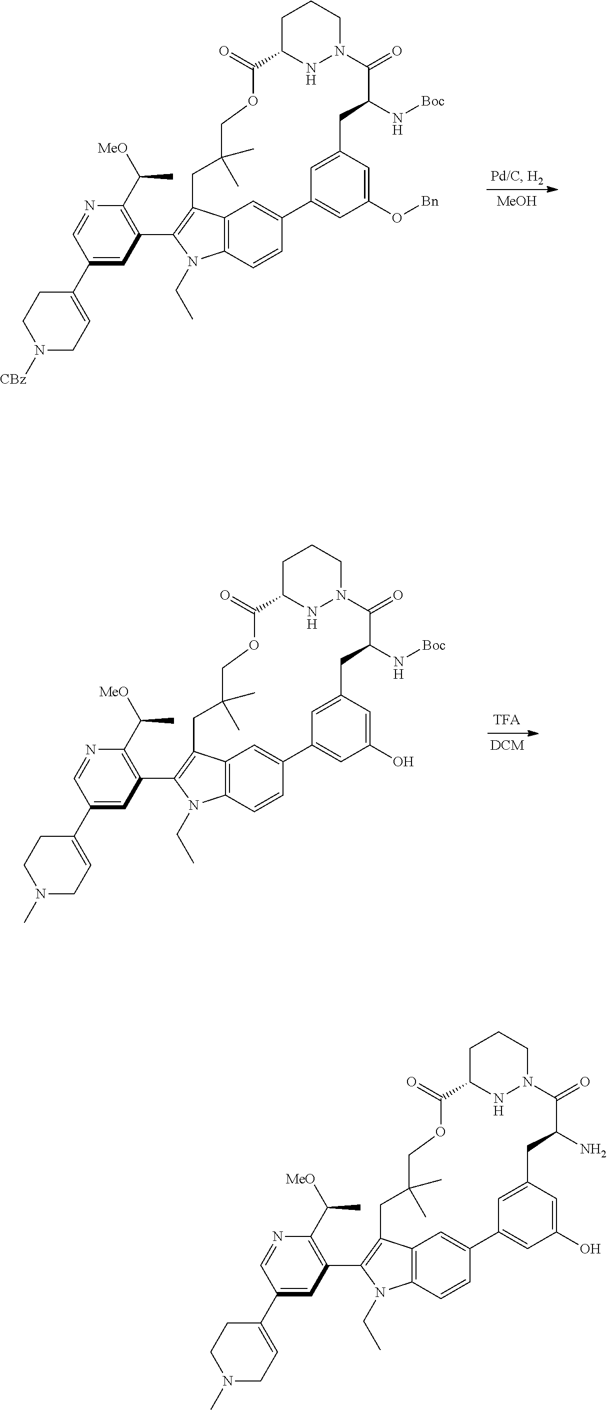

### Step 1: Synthesis of benzyl(S)-5-bromo-6-(1-methoxyethyl)-3′, 6′-dihydro-[3,4′-bipyridine]-1′ (2′H)-carboxylate

To a solution of(S)-3-bromo-5-iodo-2-(1-methoxyethyl) pyridine (6.0 g, 17.55 mmol) and benzyl 4-(4,4,5,5-tetramethyl-1,3,2-dioxaborolan-2-yl)-3,6-dihydropyridine-1 (2H)-carboxylate (7.23 g, 21.05 mmol) in dioxane (70 mL) and H₂O (14 mL) was added K₂CO₃ (6.06 g, 43.86 mmol) and Pd(dppf)Cl₂ (1.28 g, 1.76 mmol). The reaction mixture was heated to 60° C. and stirred for 3 h. The mixture was diluted with H₂O (50 mL) then extracted into EtOAc (3×100 mL). The combined organic layers were washed with brine (3×50 mL), dried over Na₂SO₄, filtered, and concentrated under reduced pressure. Purification by silica gel column chromatography (25% EtOAc/pet. ether) afforded the desired product (7.1 g, 94% yield). LCMS (ESI) m/z: [M+H] calcd for C₂₁H₂₃BrN₂O₃: 431.10; found 431.1.

### Step 2: Synthesis of tert-butyl ((6³S,4S)-25-(benzyloxy)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a solution of tert-butyl ((6³S,4S)-25-(benzyloxy)-12-iodo-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (5.0 g, 6.31 mmol), Pd₂(dba)₃ (690 mg, 757 μmol), S-Phos (0.78 g, 1.89 mmol), and KOAc (2.17 g, 22.08 mmol) in toluene (75 mL) was added 4,4,5,5-tetramethyl-1,3,2-dioxaborolane (5.65 g, 44.15 mmol). The reaction mixture was heated to 60° C. and stirred for 3 h. The reaction was quenched with H₂O at 0° C. then extracted into EtOAc (3×100 mL). The combined organic layers were washed with brine (3×30 mL), dried over Na₂SO₄, filtered, and concentrated under reduced pressure. Purification by silica gel column chromatography (50% EtOAc/pet. ether) afforded the desired product (4.5 g, 90% yield). LCMS (ESI) m/z: [M+H] calcd for C₇₅H₅₇BN₄O₈: 793.43; found 793.4.

### Step 3: Synthesis of benzyl 5-((6³S,4S)-25-(benzyloxy)-4-((tert-butoxycarbonyl) amino)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl)-3′, 6′-dihydro-[3,4′-bipyridine]-1′ (2′H)-carboxylate

To a solution of tert-butyl ((6³S,4S)-25-(benzyloxy)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (4.0 g, 5.05 mmol) and benzyl(S)-5-bromo-6-(1-methoxyethyl)-3′, 6′-dihydro-[3,4′-bipyridine]-1′ (2′H)-carboxylate (2.61 g, 6.06 mmol) in dioxane (50 mL) and H₂O (10 mL) was added K₂CO₃ (1.74 g, 12.6 mmol) and Pd(dtbpf)Cl₂ (330 mg, 505 μmol). The reaction mixture was heated to 70° C. After 3 h the reaction was diluted with H₂O (40 mL) and extracted into EtOAc (3×100 mL). The combined organic layers were washed with brine (2×50 mL), dried over Na₂SO₄, filtered, and concentrated under reduced pressure. Purification by silica gel column chromatography (50% EtOAc/pet. ether) afforded the desired product (4.1 g, 80% yield). LCMS (ESI) m/z: [M+H] calcd for C₆₀H₆₈N₆O₉: 1017.51; found 1017.4.

### Step 4: Synthesis of benzyl 5-((6³S,4S)-25-(benzyloxy)-4-((tert-butoxycarbonyl) amino)-11-ethyl-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl)-3′, 6′-dihydro-[3,4′-bipyridine]-1′ (2′H)-carboxylate

To a solution of benzyl 5-((6³S,4S)-25-(benzyloxy)-4-((tert-butoxycarbonyl) amino)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl)-3′, 6′-dihydro-[3,4′-bipyridine]-1′ (2′H)-carboxylate (4.0 g, 3.93 mmol) and CS₂CO₃ (3.84 g, 11.80 mmol) in DMF (30 mL) at 0° C. was added iodoethane (2.45 g, 15.73 mmol). The reaction mixture was warmed to room temperature. After 3 h the reaction mixture was diluted with H₂O (100 mL) and extracted into EtOAc (3×200 mL). The combined organic layers were washed with brine (3×100 mL), dried over Na₂SO₄, filtered, and concentrated under reduced pressure. Purification by silica gel column chromatography (66% EtOAc/pet. ether) afforded the desired product (1.4 g, 34% yield). LCMS (ESI) m/z: [M+H] calcd for C₆₂H₇₂N₆O₉: 1045.54; found 1045.5.

### Step 5: Synthesis of tert-butyl ((6³S,4S)-1¹-ethyl-25-hydroxy-1²-(2-((S)-1-methoxyethyl)-5-(1-methylpiperidin-4-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

A solution of benzyl 5-((6³S,4S)-25-(benzyloxy)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl)-3′, 6′-dihydro-[3,4′-bipyridine]-1′ (2′H)-carboxylate (1.29 g, 1.23 mmol) and Pd/C (700 mg) in MeOH (30 mL) was stirred for 72 h at room temperature under H₂ atmosphere. The reaction mixture was then filtered with MeOH (3×50 mL). The filtrate was concentrated under reduced pressure which afforded the desired product (850 mg, crude). LCMS (ESI) m/z: [M+H] calcd for C₄₈H₆₄N₆O₇: 837.49; found 837.7.

### Step 6: Synthesis of (6³S,4S)-4-amino-1¹-ethyl-25-hydroxy-1²-(2-((S)-1-methoxyethyl)-5-(1-methylpiperidin-4-yl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a solution of tert-butyl ((6³S,4S)-1¹-ethyl-25-hydroxy-1²-(2-((S)-1-methoxyethyl)-5-(1-methylpiperidin-4-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (840 mg, 1.00 mmol) in DCM (10 mL) at 0° C. was added TFA (3.0 mL, 40.4 mmol). The reaction mixture was warmed to room temperature. After 2 h the reaction was cooled to 0° C., quenched with sat. at. NaHCO₃, and extracted into EtOAc (3×50 mL). The combined organic layers were washed with brine (2×30 mL), dried over Na₂SO₄, filtered, and concentrated under reduced pressure which afforded product (670 mg, crude). LCMS (ESI) m/z: [M+H] calcd for C₄₃H₅₆N₆O₅: 737.44; found 737.3.

### Intermediate 26. Synthesis of (63S, 4S)-4-amino-1¹-ethyl-25-hydroxy-1²-(2-((S)-1-methoxyethyl)-5-(4-methylpiperazin-1-yl) pyridine-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

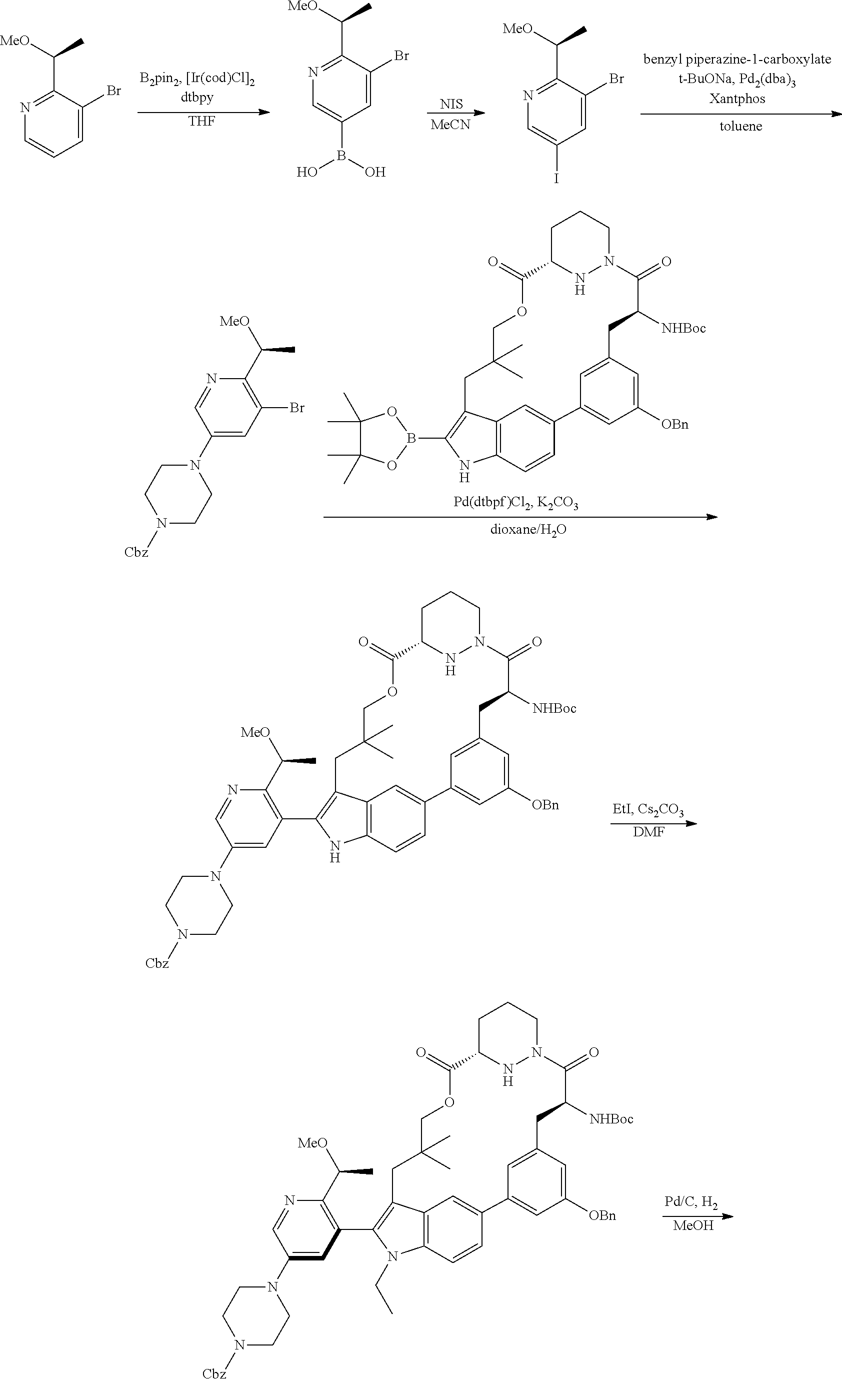

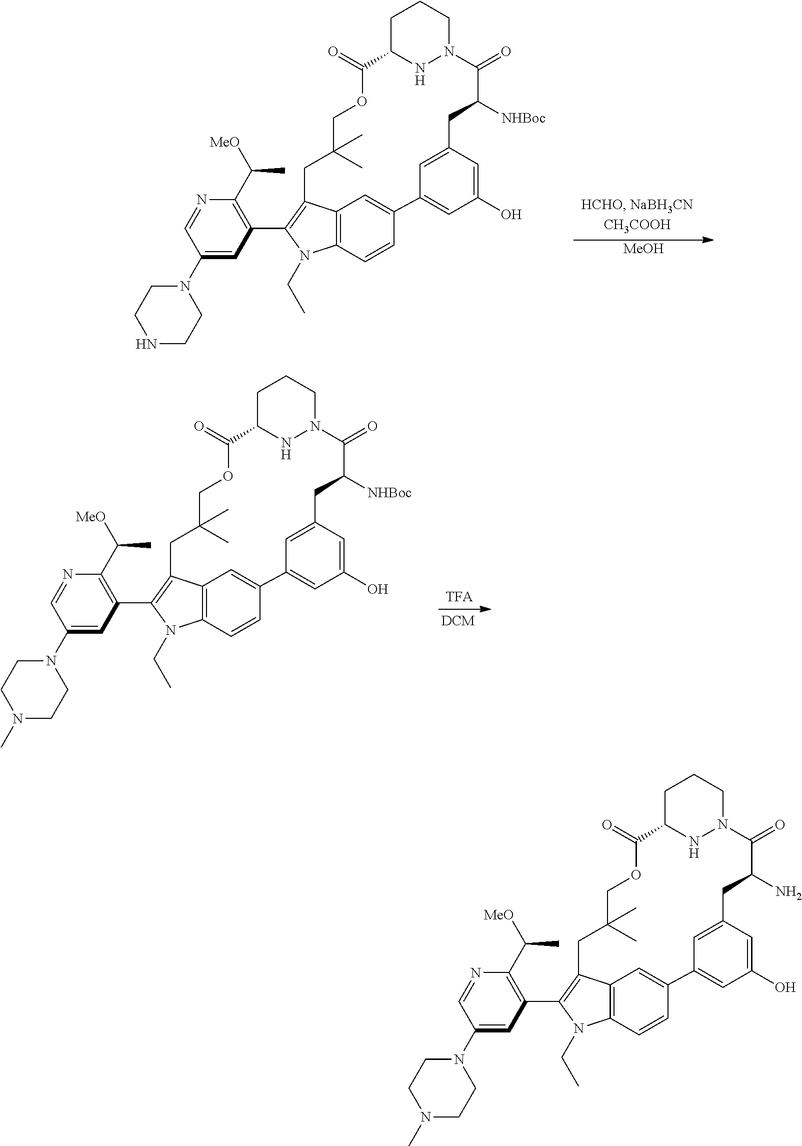

### Step 1: Synthesis of(S)-(5-bromo-6-(1-methoxyethyl) pyridin-3-yl) boronic acid

To a solution of(S)-3-bromo-2-(1-methoxyethyl) pyridine (40 g, 185 mmol) and bis (pinacolato)diboron (70.5 g, 278 mmol) in THF (1.6 L) at 75° C. was added 4,4′-di-tert-butyl-2,2′-bipyridine (7.45 g, 27.7 mmol) and [Ir(cod)Cl]₂ (1.24 mg, 1.85 mmol). After 16 h the mixture was concentrated under reduced pressure and the residue diluted with H₂O (1 L). The aqueous layer extracted with DCM/MeOH (2 L, 5:1), dried with Na₂SO₄, filtered, and concentrated under reduced pressure. Following purification by reverse phase chromatography (10→50% MeCN/H₂O, 0.1% HCO₂H) the combined product fractions were partially concentrated under reduced pressure. The aqueous layer was extracted with DCM/MeOH (3000 mL, 5:1), dried with Na₂SO₄, filtered, and concentrated under reduced pressure to afford the desired product (35.0 g, 65.5% yield). LCMS (ESI) m/z: [M+Na] calcd for C₈H₁₁BBrNO₃: 282.00; found 281.1.

### Step 2: Synthesis of(S)-3-bromo-5-iodo-2-(1-methoxyethyl) pyridine

To a solution of(S)-(5-bromo-6-(1-methoxyethyl) pyridin-3-yl) boronic acid (35.0 g, 135 mmol) in MeCN (100 mL) was added N-iodosuccinimide (60.6 g, 269 mmol). The resulting reaction mixture was stirred overnight and then concentrated under reduced pressure. Purification by normal phase chromatography (10% EtOAc/pet. ether) afforded the desired product (40.0 g, 78.1% yield). LCMS (ESI) m/z: [M+H] calcd for C₈H₉BrINO: 341.90; found 341.8.

### Step 3: Synthesis of benzyl(S)-4-(5-bromo-6-(1-methoxyethyl) pyridin-3-yl) piperazine-1-carboxylate

To a solution of(S)-3-bromo-5-iodo-2-(1-methoxyethyl) pyridine (7.0 g, 20.5 mmol) and benzyl piperazine-1-carboxylate (9.0 g, 40.8 mmol) in toluene (70 mL) were added Pd₂(dba)₃ (375 mg, 0.409 mmol), Xantphos (1.18 g, 2.05 mmol) and sodium tert-butoxide (2.29 g, 24.6 mmol). The resulting mixture was heated to 120° C. and stirred for 16 h then cooled to room temperature and concentrated under reduced pressure. Purification by normal phase chromatography (25% EtOAc/pet. ether) afforded the desired product (5.0 g, 50.6% yield). LCMS (ESI) m/z: [M+H] calcd for C₂₀H₂₄BrN₃O₃: 434.11; found 434.0.

### Step 4: Synthesis of benzyl 4-(5-((6³S,4S)-25-(benzyloxy)-4-((tert-butoxycarbonyl) amino)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate

To a solution of benzyl(S)-4-(5-bromo-6-(1-methoxyethyl) pyridin-3-yl) piperazine-1-carboxylate (3.29 g, 7.56 mmol) and tert-butyl ((6³S,4S)-25-(benzyloxy)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (50 g, 6.31 mmol) dioxane (40 mL) and H₂O (10 mL) were added K₂CO₃ (1.74 g, 12.614 mmol) and Pd(dtbpf)Cl₂ (822 mg, 1.26 mmol) and the resulting mixture was heated to 80° C. for 2 h. The reaction mixture was then concentrated under reduced pressure and diluted with H₂O (1 L). The aqueous layer was extracted with EtOAc (3×200 mL) and the combined organic layers were washed with H₂O, dried with Na₂SO₄, filtered, and concentrated under reduced pressure. Purification by normal phase chromatography (50% EtOAc/pet. ether) afforded the desired product (5.0 g, 73.8% yield). LCMS (ESI) m/z: [M+H] calcd for C₅₉H₆₉N₇O₉: 1020.54; found 1020.6.

### Step 5: Synthesis of benzyl 4-(5-((63S, 4S)-25-(benzyloxy)-4-((tert-butoxycarbonyl) amino)-11-ethyl-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate

To a stirred solution of benzyl 4-(5-((6³S,4S)-25-(benzyloxy)-4-((tert-butoxycarbonyl) amino)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (5.0 g, 5 mmol) in DMF (50 mL) at 0° C. was added Cs₂CO₃ (3.19 g, 9.80 mmol) and ethyl iodide (1.53 g, 10 mmol). The resulting mixture was stirred for 2 h at room temperature and then diluted with H₂O (200 mL). The aqueous layer was extracted with EtOAc (3×100 mL) and the combined organic layers were washed with H₂O, dried with Na₂SO₄, filtered, and concentrated under reduced pressure. Purification by normal phase chromatography (33% EtOAc/pet. ether) afforded the desired product (1.8 g, 35% yield). LCMS (ESI) m/z: [M+H] calcd for C₆₁H₇₃N₇O₉: 1048.56; found 1048.4.

### Step 6: Synthesis of tert-butyl ((63S, 4S)-1¹-ethyl-25-hydroxy-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred solution of benzyl 4-(5-((63S, 4S)-25-(benzyloxy)-4-((tert-butoxycarbonyl) amino)-1¹-ethyl-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-12-yl)-6-((S)-1-methoxyethyl) pyridin-3-yl) piperazine-1-carboxylate (1.80 g, 1.72 mmol) in MeOH (20 mL) was added Pd/C (900 mg). The resulting mixture was stirred for 2 h at room temperature under a hydrogen atmosphere, filtered, and the filter cake washed with MeOH. The filtrate was concentrated under reduced pressure to afford the crude desired product which was used without further purification. LCMS (ESI) m/z: [M+H] calcd for C₄₆H₆₁N₇O₇: 824.47; found 824.3.

### Step 7: Synthesis of tert-butyl ((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred solution of tert-butyl ((63S, 4S)-1¹-ethyl-25-hydroxy-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate carbamate (590 mg, 0.716 mmol) and HCHO (129 mg, 1.43 mmol, 37 wt % in H₂O) in MeOH (6 ml) at 0° C. were added CH₃COOH (122 mg, 2.02 mmol) and NaBH₃CN (85.3 mg, 1.35 mmol). The resulting mixture was warmed to room temperature and stirred for 2 h. The reaction mixture was then concentrated under reduced pressure and diluted with H₂O (100 mL). The aqueous layer was extracted with EtOAc (3×100 mL) and the combined organic layers were washed with H₂O, dried with Na₂SO₄, filtered, concentrated under reduced pressure. pressure to afford the crude desired product which was used without further purification. LCMS (ESI) m/z: [M+H] calcd for C₄₇H₆₃N₇O₉: 838.49; found 838.4.

### Step 8: Synthesis of (63S, 4S)-4-amino-1¹-ethyl-25-hydroxy-1²-(2-((S)-1-methoxyethyl)-5-(4-methylpiperazin-1-yl) pyridine-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a stirred solution of tert-butyl ((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (590 mg, 0.704 mmol) in DCM (6 mL) at 0° C. was added TFA (3.0 mL). The resulting mixture was stirred for 30 min and then concentrated under reduced pressure to afford the crude desired product which was used without further purification. LCMS (ESI) m/z: [M+H] calcd for C₄₂H₅₅N₇O₅: 738.44; found 738.4.

### Intermediate 27. Synthesis of (6³S,4S,Z)-4-amino-1¹-ethyl-1²-(2-((S)-1-methoxyethyl)-5-(4-methylpiperazin-1-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione

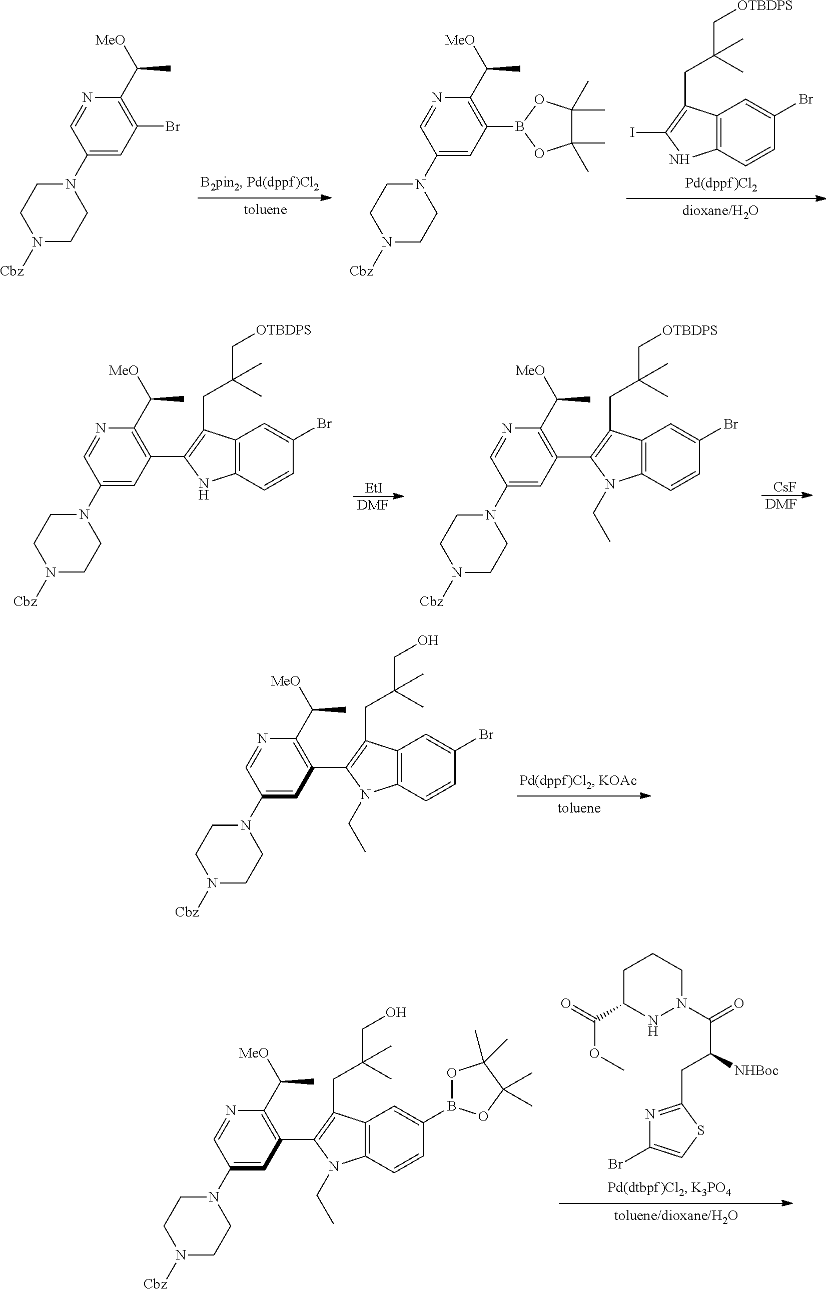

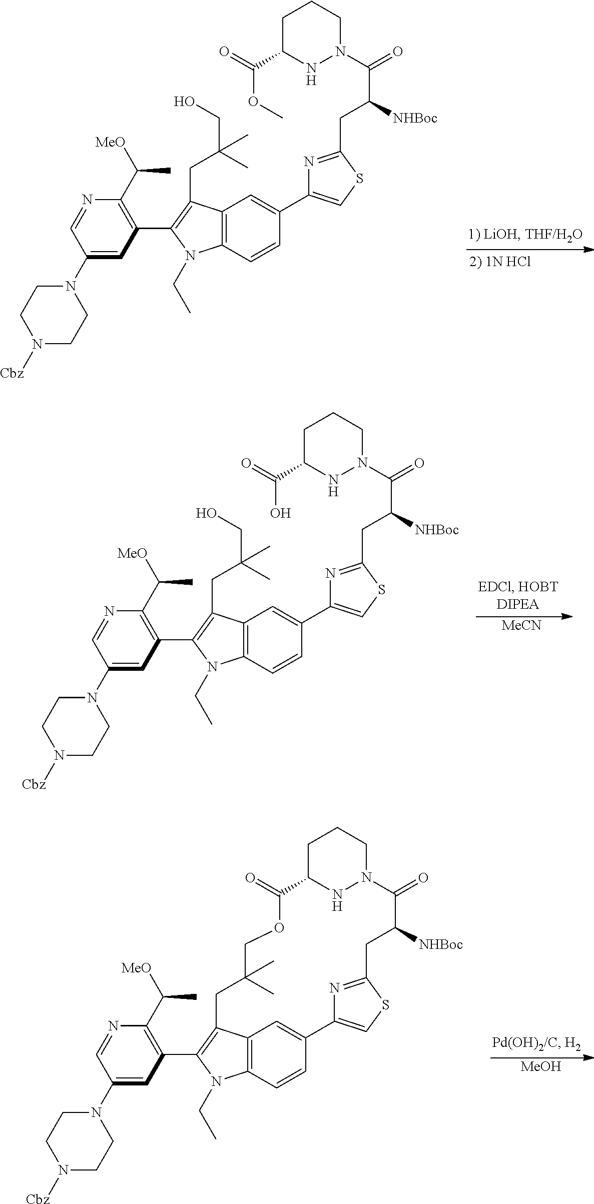

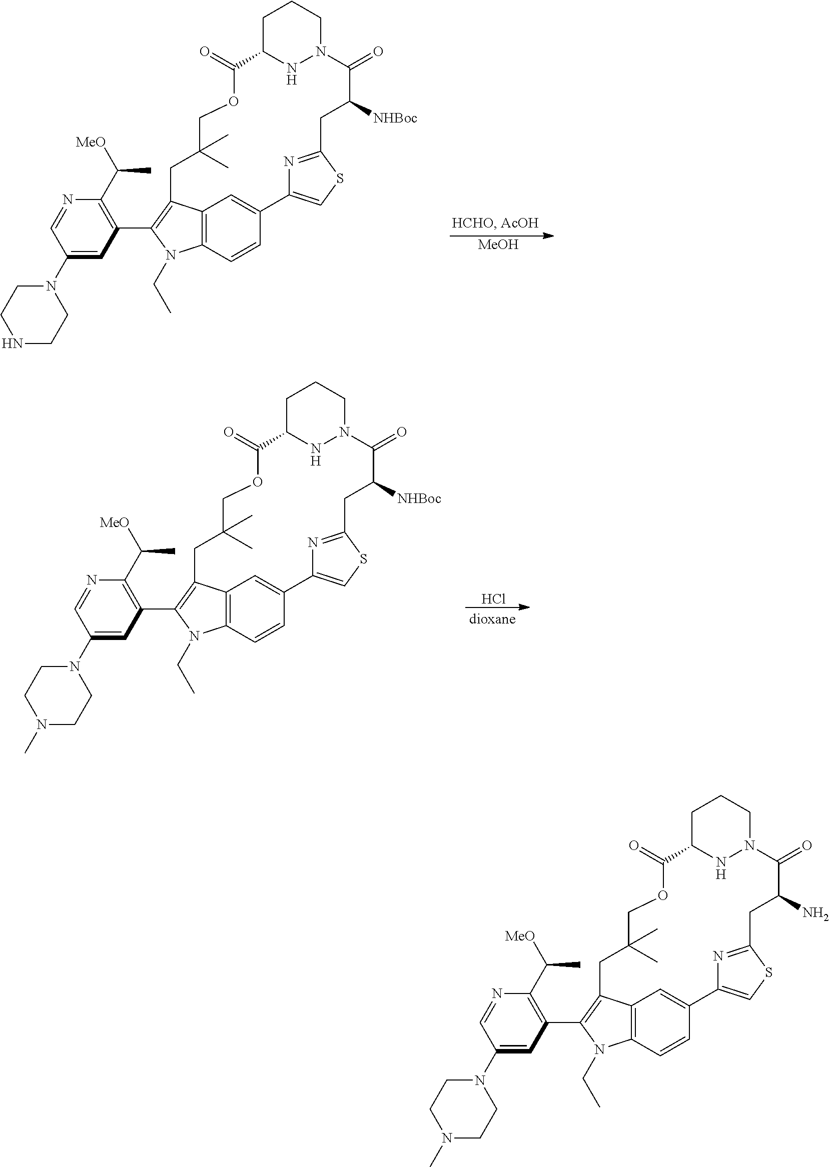

### Step 1: Synthesis of benzyl(S)-4-(5-bromo-6-(1-methoxyethyl) pyridin-3-yl) piperazine-1-carboxylate

Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.821 mmol), bis(pinacolato)diboron (86.82 g, 341.903 mmol), Pd(dppf)Cl₂ (22.74 g, 31.082 mmol), KOAc (76.26 g, 777.052 mmol), and toluene (1 L). The resulting solution was stirred for 2 days at 90° C. in an oil bath. The reaction mixture was cooled to room temperature. The resulting mixture was concentrated under reduced pressure. The residue was purified by neutral alumina column chromatography (25% EtOAc/hexanes) to afford the desired product (167 g, crude). LCMS (ESI) m/z: [M+H] calcd for C₂₆H₃₆BN₃O₅: 481.3; found 482.1.

### Step 2: Synthesis of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate

Into a 3-L 3-necked round-bottom flask purged and maintained with an inert atmosphere of argon, was placed(S)-4-(6-(1-methoxyethyl)-5-(4,4,5,5-tetramethyl-1,3,2-dioxaborolan-2-yl) pyridin-3-yl) piperazine-1-carboxylate (167 g, 346.905 mmol), 5-bromo-3-[3-[(tert-butyldiphenylsilyl)oxy]-2,2-dimethylpropyl]-2-iodo-1H-indole (224.27 g, 346.905 mmol), Pd(dppf)Cl₂ (25.38 g, 34.69 mmol), dioxane (600 mL), H₂O (200 mL), K₃PO₄ (184.09 g, 867.262 mmol), and toluene (200 mL). The resulting solution was stirred for overnight at 70° C. in an oil bath. The reaction mixture was cooled to room temperature after reaction completed. The resulting mixture was concentrated under reduced pressure. The residue was purified by normal phase column chromatography (50% EtOAc/hexanes) to afford the desired product (146 g, 48.2% yield). LCMS (ESI) m/z: [M+H] calcd for C₄₉H₅₇BrN₄O₄Si: 872.3; found 873.3.

### Step 3: Synthesis of benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate

To a stirred mixture of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (146 g, 167.047 mmol) and Cs₂CO₃ (163.28 g, 501.14 mmol) in DMF (1200 mL) was added C₂H₅I (52.11 g, 334.093 mmol) in portions at 0° C. under N₂ atmosphere. The final reaction mixture was stirred at room temperature for 12 h. The resulting mixture was diluted with EtOAc (1 L) and washed with brine (3×1.5L). The organic layers were dried over anhydrous Na₂SO₄. After filtration, the filtrate was concentrated under reduced pressure to afford the desired product (143 g, crude). LCMS (ESI) m/z: [M+H] calcd for C₅₁H₆₁BrN₄O₄Si: 900.4; found 901.4.

### Step 4: Synthesis of benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate

To a stirred mixture of benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate (143 g, 158.526 mmol) in DMF (1250 mL) was added CsF (72.24 g, 475.578 mmol). The reaction mixture was stirred at 60° C. for 2 days under N₂ atmosphere. The resulting mixture was diluted with EtOAc (1 L) and washed with brine (3×1 L). The organic phase was concentrated under reduced pressure and the residue was purified by silica gel column chromatography to afford two atropisomers of benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (38 g, 36% yield, RT=1.677 min in 3 min LCMS (0.1% FA)) and B (34 g, 34% yield, RT=1.578 min in 3 min LCMS (0.1% FA)). LCMS (ESI) m/z: [M+H] calcd for C₃₅H₄₃BrN₄O₄: 663.2; found 662.2.

### Step 5: Synthesis of benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate

Into a 500-mL 3-necked round-bottom flask purged and maintained with an inert atmosphere of nitrogen, was placed benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (14 g, 21.095 mmol), bis(pinacolato)diboron (5.89 g, 23.205 mmol), Pd(dppf)Cl₂ (1.54 g, 2.11 mmol), KOAc (5.18 g, 52.738 mmol), and toluene (150 mL). The resulting solution was stirred for 5 h at 90° C. in an oil bath. The reaction mixture was then cooled to room temperature. The resulting mixture was concentrated under reduced pressure and the residue was purified by silica gel column chromatography to afford the desired product (12 g, 76.0% yield). LCMS (ESI) m/z: [M+H] calcd for C₄₁H₅₅BN4O₆: 710.4; found 711.3.

### Step 6: Synthesis of methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate

Into a 250-mL round-bottom flask purged and maintained with an inert atmosphere of argon, was placed benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (10.8 g, 15.196 mmol), methyl (3S)-1-[(2S)-3-(4-bromo-1,3-thiazol-2-yl)-2-[(tert-butoxycarbonyl) amino]propanoyl]-1,2-diazinane-3-carboxylate (7.98 g, 16.716 mmol), Pd(dtbpf)Cl₂ (0.99 g, 1.52 mmol), K₃PO₄ (8.06 g, 37.99 mmol), toluene (60 mL), dioxane (20 mL), and H₂O (20 mL). The resulting solution was stirred for 3 h at 70° C. in an oil bath. The reaction mixture was then cooled to room temperature. The resulting solution was extracted with EtOAc (2×50 mL) and concentrated under reduced pressure. The residue was purified by normal phase column chromatography to afford the desired product (8 g, 50.9% yield). LCMS (ESI) m/z: [M+H] calcd for C₅₂H₆₈N₈O₉S: 980.5; found 980.9.

### Step 7: Synthesis of(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid

To a stirred mixture of methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate (12 g, 12.230 mmol) in THF (100 mL) and H₂O (100 mL) was added LiOH (2.45 g, 61.148 mmol) under N₂ atmosphere and the resulting mixture was stirred for 2 h at room temperature. The resulting mixture was concentrated under reduced pressure and the pH of aqueous phase was acidified to 5 with HCl (1N) at 0° C. The aqueous layer was extracted with DCM (3×100 mL). The organic phase was concentrated under reduced pressure to afford the desired product (10 g, 84.5% yield). LCMS (ESI) m/z: [M+H] calcd for C₅₁H₆₆N₈O₉S: 966.5; found 967.0.

### Step 8: Synthesis of benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl) piperazine-1-carboxylate

Into a 3-L round-bottom flask purged and maintained with an inert atmosphere of nitrogen, was placed(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid (18 g, 18.61 mmol), MeCN (1.8 L), DIPEA (96.21 g, 744.417 mmol), EDCI (107.03 g, 558.313 mmol), HOBT (25.15 g, 186.104 mmol). The resulting solution was stirred for overnight at room temperature. The resulting mixture was concentrated under reduced pressure after reaction completed. The resulting solution was diluted with DCM (1 L) and was washed with HCl (3×1 L, 1N aqueous). The resulting mixture was washed with H₂O (3×1 L) and then the organic layer was concentrated. The residue was purified by normal phase column chromatography (50% EtOAc/hexanes) to afford the desired product (10.4 g, 54.9% yield). LCMS (ESI) m/z: [M+H] calcd for C₅₁H₆₄N₈O₈S: 948.5; found 949.3.

### Step 9: Synthesis of tert-butyl ((6³S,4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

Into a 250-mL round-bottom flask purged and maintained with an inert atmosphere of nitrogen, was placed benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl) piperazine-1-carboxylate (10.40 g, 10.957 mmol), Pd (OH) 2/C(5 g, 46.984 mmol), and MeOH (100 mL). The resulting solution was stirred for 3 h at room temperature under a 2 atm H₂ atmosphere. The solids were filtered out and the filter cake was washed with MeOH (3×100 mL). The combined organic phase was concentrated under reduced pressure to afford the desired product (8.5 g, 90.4% yield). LCMS (ESI) m/z: [M+H] calcd for C₄₃H₅₈N₈O₆S: 814.4; found 815.3.

### Step 10: Synthesis of tert-butyl ((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

Into a 1000-mL round-bottom flask purged and maintained with an inert atmosphere of nitrogen, was placed tert-butyl ((6³S,4S,Z)-1¹-ethyl-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.5 g, 10.429 mmol), MeOH (100 mL), AcOH (1.88 g, 31.286 mmol) and stirred for 15 min. Then HCHO (1.88 g, 23.15 mmol, 37% aqueous solution) and NaBH₃CN (788 mg, 12.5 mmol) was added at room temperature. The resulting solution was stirred for 3 hr. The resulting mixture was quenched with H₂O (100 mL) and concentrated under reduced pressure to remove MeOH. The resulting solution was diluted with DCM (300 mL) and was washed with H₂O (3×100 mL). The organic phase was concentrated under reduced pressure to afford the desired product (8.2 g, 90.1% yield). LCMS (ESI) m/z: [M+H] calcd for C₄₄H₆₀N₈OBS: 828.4; found 829.3.

### Step 10: Synthesis of (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione

Into a 250-mL round-bottom flask purged and maintained with an inert atmosphere of nitrogen, was placed tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.20 g, 9.891 mmol) and dioxane (40 mL), followed by the addition of HCl in 1,4-dioxane (4M, 40 mL) at 0° C. The resulting solution was stirred for 1 h at 0° C. The mixture was then concentrated under reduced pressure. The resulting solution was diluted with DCM (600 mL) and sat. aq. NaHCO₃ (400 mL). The organic phase was then washed twice with brine (500 mL). The organic phase was concentrated under reduced pressure to afford the desired product (7.2 g, 94.9% yield).

### Intermediate 28. Synthesis of (6³S,4S)-4-amino-25-(difluoromethyl)-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

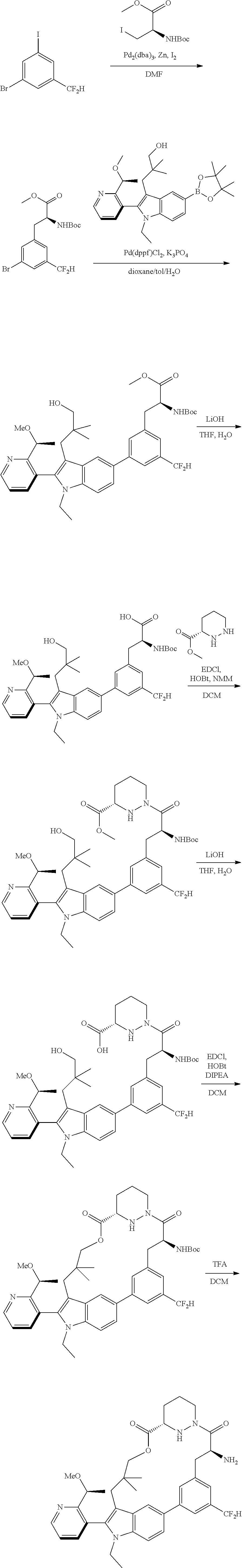

### Step 1: Synthesis of methyl(S)-3-(3-bromo-5-(difluoromethyl)phenyl)-2-((tert-butoxycarbonyl) amino) propanoate

Into a 1000 mL 3-necked round-bottom flask was added Zn powder (43.42 g, 663.835 mmol) and 12 (1.30 g, 5.106 mmol) in DMF (400 mL) at room temperature. To the above mixture was added a solution of methyl (2R)-2-[(tert-butoxycarbonyl) amino]-3-iodopropanoate (36.42 g, 110.64 mmol) in DMF (10 mL). The mixture was heated to 30° C. for 10 min. To the mixture was then added a solution of methyl (2R)-2-[(tert-butoxycarbonyl) amino]-3-iodopropanoate (72.83 g, 221.28 mmol) in DMF (20 mL) dropwise at room temperature. The resulting mixture was stirred for 30 min. The resulting mixture was filtered and the solution was added to a mixture of 1-bromo-3-(difluoromethyl)-5-iodobenzene (85.0 g, 255.321 mmol), tris (furan-2-yl) phosphane (3.56 g, 15.319 mmol), and Pd₂(dba)₃ (4.68 g, 5.106 mmol) in DMF (400 mL) at room temperature under argon atmosphere. The reaction mixture was heated to 60° C. for 10 min and was then removed from the oil bath and was stirred for 1 h until the temperature of the resulting mixture cooled down to 50° C. The reaction was quenched with aq. NH₄Cl (3000 mL) and the aqueous layer was extracted with EtOAc (3×1000 mL). The combined organic layers were washed with brine (2×1000 mL), dried over anhydrous Na₂SO₄, filtered, and concentrated under reduced pressure. The residue was purified by silica gel column chromatography (9% EtOAc/pet. ether) to afford the desired product (59 g, 56.6% yield).

### Step 2: Synthesis of methyl(S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoate

A mixture of methyl (2S)-3-[3-bromo-5-(difluoromethyl)phenyl]-2-[(tert-butoxycarbonyl) amino]propanoate (90.0 g, 220.459 mmol), (S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (1.50 g, 3.046 mmol), Pd(dppf)Cl₂ (16.13 g, 22.046 mmol) and K₃PO₄ (116.99 g, 551.148 mmol) in dioxane (600 mL), H₂O (200 mL), and toluene (200 mL) was stirred for 2 h at 70° C. The resulting mixture was concentrated under reduced pressure and then diluted with H₂O (300 mL). The mixture was extracted with EtOAc (3×500 mL). The combined organic layers were washed with H₂O (3×500 mL), dried over anhydrous Na₂SO₄. After filtration, the filtrate was concentrated under reduced pressure and the residue was purified by silica gel column chromatography (50% EtOAc/pet. ether) to afford the desired product (128 g, 83.7% yield). LCMS (ESI) m/z: [M+H] calcd for C₃₉H₄₉F₂N₃O₆: 694.37; found 694.2.

### Step 3: Synthesis of(S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoic acid

To a stirred solution of methyl(S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoate (125.0 g, 180.159 mmol) in THF (800 mL) was added LiOH·H₂O (11.48 g, 479.403 mmol) in H₂O (200 mL) dropwise at 0° C. The resulting mixture was stirred for 2 h at 0° C. The mixture was acidified to pH 6 with 1 M HCl (aq.) and was then extracted with EtOAc (3×800 mL). The combined organic layers were washed with brine (2×200 mL), dried over anhydrous Na₂SO₄. After filtration, the filtrate was concentrated under reduced pressure to afford the desired product (125 g, crude). LCMS (ESI) m/z: [M+H] calcd for C₃₈H₄₇F2N₃O₆: 680.37; found 680.2.

### Step 4: Synthesis of methyl(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylate

To a stirred solution of methyl (3S)-1,2-diazinane-3-carboxylate (39.77 g, 275.814 mmol) and NMM (185.98 g, 1838.760 mmol) in DCM (1500 mL) was(S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoic acid (125.0 g, 183.876 mmol), HOBt (12.42 g, 91.938 mmol) and

EDCI (70.50 g, 367.752 mmol) in portions at 0° C. The resulting mixture was stirred at room temperature for 16 h. The reaction mixture was then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet.ether) to afford the desired product (110 g, 74.2% yield). LCMS (ESI) m/z: [M+H] calcd for C₄₄H₅₇F₂N₅O₇: 806.43; found 806.2.

### Step 5: Synthesis of(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylic acid

To a stirred solution of methyl(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylate (110.0 g, 136.482 mmol) in THF (800 mL) was added a solution of LiOH·H₂O (17.18 g, 409.446 mmol) in H₂O (200 mL) in portions at 0° C. The resulting mixture was stirred for 2 h at 0° C. and was then neutralized to pH 6 with 0.5 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (100 g, crude). LCMS (ESI) m/z: [M+H] calcd for C₄₃H₅₅F₂N₅O₇: 792.42; found 792.4.

### Step 6: Synthesis of tert-butyl ((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred solution of(S)-1-((S)-2-((tert-butoxycarbonyl) amino)-3-(3-(difluoromethyl)-5-(1-ethyl-3-(3-hydroxy-2,2-dimethylpropyl)-2-(2-((S)-1-methoxyethyl) pyridin-3-yl)-1H-indol-5-yl)phenyl) propanoyl) hexahydropyridazine-3-carboxylic acid (100.0 g, 126.273 mmol) in DCM (6000 mL) was added DIPEA (163.20 g, 1262.730 mmol), HOBt (85.31 g, 631.365 mmol), and EDCI (363.10 g, 1894.095 mmol) dropwise at 0° C. The resulting mixture was stirred overnight at room temperature. The mixture was then washed with 0.5 M HCl (2 ×2 000 mL) and brine (2×2000 mL), dried over anhydrous Na₂SO₄, filtered, and concentrated under reduced pressure. The residue was purified by silica gel column chromatography (50% EtOAc/pet. ether) to afford the desired product (70 g, 71.6% yield). LCMS (ESI) m/z: [M+H] calcd for C₄₃H₅₃F₂N₅O₆: 774.41; found 774.0.

### Step 7: Synthesis of (6³S,4S)-4-amino-25-(difluoromethyl)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

To a stirred solution of tert-butyl ((6³S,4S)-25-(difluoromethyl)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (202.0 mg, 0.261 mmol) in DCM (2 mL) was added TFA (1.0 mL) dropwise at 0° C. The resulting mixture was stirred for 1.5 h at 0° C. and was then concentrated under reduced pressure to afford the desired product. LCMS (ESI) m/z: [M+H] calcd for C₃₈H₄₅F₂N₅O₄: 674.35; found 674.5.

### Intermediate 29. Synthesis of (6³S,4S)-4-amino-1¹-ethyl-25-(fluoromethyl)-12-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione

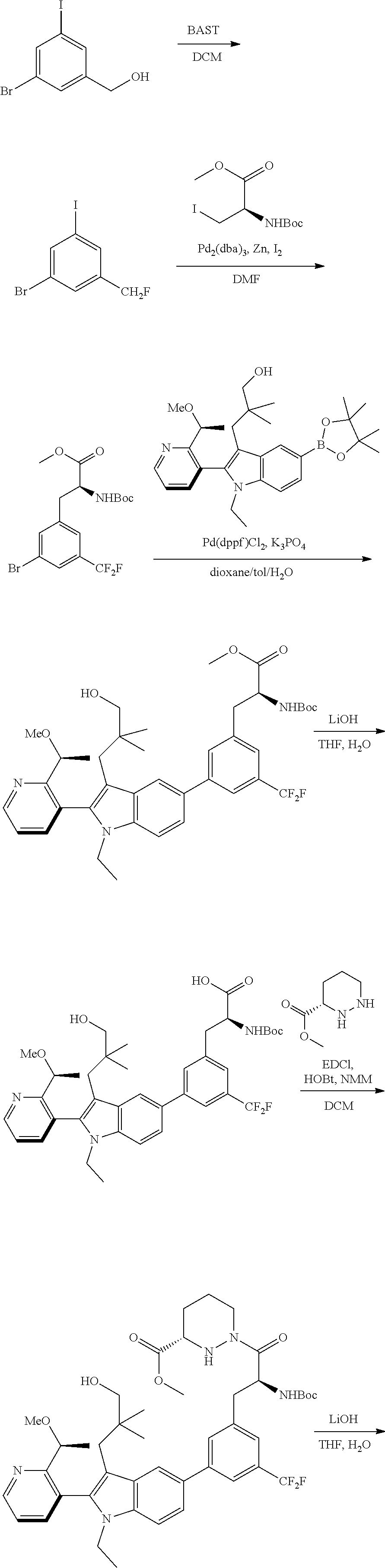

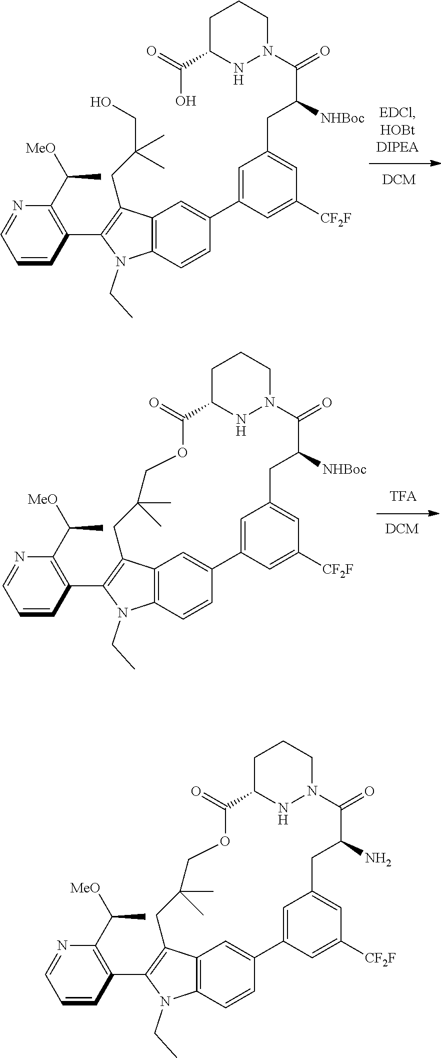

### Step 1: Synthesis of 1-bromo-3-(fluoromethyl)-5-iodobenzene

To a solution of (3-bromo-5-iodophenyl) methanol (175.0 g, 559.227 mmol) in DCM (2 L) was added BAST (247.45 g, 1118.454 mmol) dropwise at 0° C. The resulting mixture was stirred for 16 h at room temperature. The reaction was quenched with sat. aq. NaHCO₃ at 0° C. The organic layers were washed with H₂O (3×700 mL) and dried over anhydrous Na₂SO₄. After filtration, the filtrate was concentrated under reduced pressure. The residue was purified by silica gel column chromatography (3% EtOAc/pet. ether) to afford the desired product (120 g, 68% yield).

### Step 2: Synthesis of methyl (2S)-3-[3-bromo-5-(fluoromethyl)phenyl]-2-[(tert-butoxycarbonyl) amino]propanoate

Into a 1000 mL 3-necked round-bottom flask was added Zn powder (32.40 g, 495.358 mmol) in DMF (350.0 mL) and 12 (967.12 mg, 3.810 mmol). To the mixture was added a solution of methyl (2R)-2-[(tert-butoxycarbonyl) amino]-3-iodopropanoate (27.0 g, 82.03 mmol) in DMF (10 mL). The mixture was heated to 30° C. for 10 min. To the mixture was then added a solution of methyl (2R)-2-[(tert-butoxycarbonyl) amino]-3-iodopropanoate (54.0 g, 164.07 mmol) in DMF (20 mL). The resulting mixture was stirred for 30 min at room temperature and was filtered. The resulting solution was added to a mixture of 1-bromo-3-(fluoromethyl)-5-iodobenzene (60 g, 190.522 mmol), tris (furan-2-yl) phosphane (2.65 g, 11.431 mmol), and Pd₂(dba)₃ (3.49 g, 3.810 mmol) in DMF (400 mL) at room temperature under argon atmosphere and the reaction mixture was heated to 60° C. for 10 min then removed the oil bath. The resulting mixture was stirred for about 1 h until the temperature cooled down to 50° C. The reaction was quenched with aq. NH₄Cl (3000 mL) and the resulting mixture was extracted with EtOAc (3×1000 mL). The combined organic layers were washed with brine (2×1000 mL) and dried over anhydrous Na₂SO₄. After filtration, the filtrate was concentrated under reduced pressure. The residue was purified by silica gel column chromatography (9% EtOAc/pet. ether) to afford the desired product (45 g, 60% yield).

### Step 3: Synthesis of methyl(S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoate

A mixture of methyl (2S)-3-[3-bromo-5-(fluoromethyl)phenyl]-2-[(tert-butoxycarbonyl) amino]propanoate (75.28 g, 192.905 mmol), (S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (95 g, 192.905 mmol), Pd(dppf)Cl₂ (14.11 g, 19.291 mmol) and K₂CO₃ (53.32 g, 385.810 mmol) in dioxane (900 mL) and H₂O (180 mL) was stirred for 2 h at 80° C. The resulting mixture was concentrated under reduced pressure and was then diluted with H₂O. The resulting mixture was extracted with EtOAc (3×1200 mL) and the combined organic layers were washed with H₂O (3×500 mL) and dried over anhydrous Na₂SO₄. After filtration, the filtrate was concentrated under reduced pressure. The residue was purified by silica gel column chromatography (50% EtOAc/pet. ether) to afford the desired product (105 g, 80% yield). LCMS (ESI) m/z: [M+H] calcd for C₃₉H₅₀FN₃O₆: 676.38; found 676.1.

### Step 4: Synthesis of(S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoic acid

To a stirred solution of methyl(S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoate (108 g, 159.801 mmol) in THF (500 mL) was added a solution of LiOH·H₂O (11.48 g, 479.403 mmol) in H₂O (500 mL) at 0° C. The resulting mixture was stirred for 2 h at 0° C. and was then acidified to pH 6 with 1 M HCl (aq.). The mixture was extracted with EtOAc (3×800 mL) and the combined organic layers were washed with brine (2×200 mL) and dried over anhydrous Na₂SO₄. After filtration, the filtrate was concentrated under reduced pressure to afford the desired product (101 g, crude). LCMS (ESI) m/z: [M+H] calcd for C₃₈H₄₈FN₃O₆: 662.36; found 662.1.

### Step 5: Synthesis of methyl(S)-1-((S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoyl) hexahydropyridazine-3-carboxylate

To a stirred solution of(S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoic acid (103 g, 155.633 mmol) and NMM (157.42 g, 1556.330 mmol) in DCM (1200 mL) was added methyl (3S)-1,2-diazinane-3-carboxylate (33.66 g, 233.449 mmol), HOBt (10.51 g, 77.816 mmol) and EDCI (59.67 g, 311.265 mmol) in portions at 0° C. The resulting mixture was stirred at room temperature for 16 h. The organic layers were then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet. ether) to afford the desired product (103 g, 83% yield). LCMS (ESI) m/z: [M+H] calcd for C₄₄H₅₈FN₅O₇: 788.44; found 788.1.

### Step 6: Synthesis of(S)-1-((S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoyl) hexahydropyridazine-3-carboxylic acid

To a stirred solution of methyl(S)-1-((S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoyl) hexahydropyridazine-3-carboxylate (103 g, 130.715 mmol) in THF (700 mL) was added a solution of LiOH·H₂O (27.43 g, 653.575 mmol) in H₂O (700 mL) at 0° C. . . . The resulting mixture was stirred for 2 h at 0° C. and was then neutralized to pH 6 with 1 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (101 g, crude). LCMS (ESI) m/z: [M+H] calcd for C₄₃H₅₆FN₅O₇: 774.43; found 774.1.

### Step 7: Synthesis of tert-butyl ((6³S,4S)-1¹-ethyl-25-(fluoromethyl)-12-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate

To a stirred solution of(S)-1-((S)-2-((tert-butoxycarbonyl) amino)-3-(3-(1-ethyl-3-(3-hydroxy-2,2-dimethylpropyl)-2-(2-((S)-1-methoxyethyl) pyridin-3-yl)-1H-indol-5-yl)-5-(fluoromethyl)phenyl) propanoyl) hexahydropyridazine-3-carboxylic acid (101 g, 130.50 mmol) in DCM (5500 mL) was added DIPEA (227.31 mL, 1305.0 mmol) and HOBt (88.17 g, 652.499 mmol), and EDCI (375.26 g, 1957.498 mmol) at 0° C. The resulting mixture was stirred at room temperature overnight. The mixture was then washed with 0.5 M HCl (2×2000 mL), brine (2×2000 mL), dried over anhydrous Na₂SO₄, filtered, and concentrated under reduced pressure. The residue was purified by silica gel column chromatography (50% EtOAc/pet. ether) to afford the desired product (68 g, 65% yield). LCMS (ESI) m/z: [M+H] calcd for C₄₃H₅₄FN₅O₆: 756.42; found 756.4.

### Step 8: Synthesis of (2S)—N-((6³S,4S)-1¹-ethyl-25-(fluoromethyl)-12-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide

To a stirred solution of tert-butyl ((6³S,4S)-1¹-ethyl-25-(fluoromethyl)-12-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) carbamate (350 mg, 0.403 mmol) in DCM (4 mL) was added TFA (1.50 mL) at 0° C. The resulting mixture was stirred at room temperature for 1.5 h and was then concentrated under reduced pressure to afford the desired product (600 mg, crude). LCMS (ESI) m/z: [M+H] calcd for C₃₈H₄₆FN_{504: 656.36}; found 656.4.

Intermediate A-1. Synthesis of N-methyl-N—((S)-1-((R)-1-tritylaziridine-2-carbonyl) pyrrolidine-3-carbonyl)-L-valine

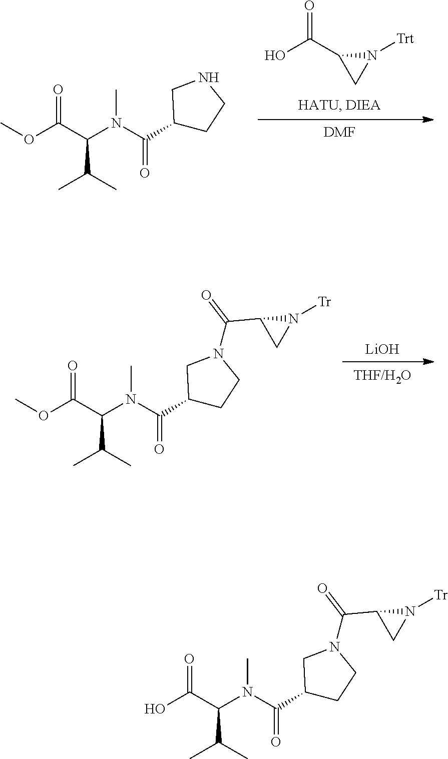

### Step 1: Synthesis of methyl N-methyl-N—((S)-1-((R)-1-tritylaziridine-2-carbonyl) pyrrolidine-3-carbonyl)-L-valinate

To a mixture of methyl N-methyl-N—((S)-pyrrolidine-3-carbonyl)-L-valinate (0.840 g, 3.47 mmol) and (R)-1-tritylaziridine-2-carboxylic acid (1.713 g, 5.2 mmol) in DMF (20 mL) at 0° C. was added DIPEA (3.0 mL, 17.33 mmol) and HATU (2.636 g, 6.93 mmol). The reaction mixture was stirred for 3 h, at which point the mixture was extracted with EtOAc (200 mL). The EtOAc layer was washed with brine (3×50 mL), dried over Na₂SO₄, filtered, and concentrated under reduced pressure. The crude residue was purified by reverse phase chromatography (10→50% MeCN/H₂O) to afford the desired product (1.02 g, 53% yield) as a solid. LCMS (ESI) m/z: [M+H] calcd for C₃₄H₃₉N₃O₄: 554.30; found 554.3.

### Step 2: Synthesis of N-methyl-N—((S)-1-((R)-1-tritylaziridine-2-carbonyl) pyrrolidine-3-carbonyl)-L-valine

To a solution of methyl N-methyl-N—((S)-1-((R)-1-tritylaziridine-2-carbonyl) pyrrolidine-3-carbonyl)-L-valinate (1.0 g, 1.81 mmol) in THF (10 mL) at 0° C. was added a solution of LiOH. H₂O (0.3789 g, 9.03 mmol) in H₂O (9.0 mL). After 3 h, the reaction solution was neutralized to pH 7 with sat. aq. NH₄Cl. The resulting mixture was extracted with EtOAc (3×50 mL) and the combined organic layers were washed with brine (3×20 mL), dried over Na₂SO₄, filtered, and concentrated under reduced pressure to afford the crude product (740 mg, 75.9% yield) as a solid. LCMS (ESI) m/z: [M
H] calcd for C₃₃H₃₇N₃O₄: 538.27; found 538.2.

Intermediate A-2. Synthesis of N-methyl-N—((S)-1-((S)-1-tritylaziridine-2-carbonyl) pyrrolidine-3-carbonyl)-L-valine

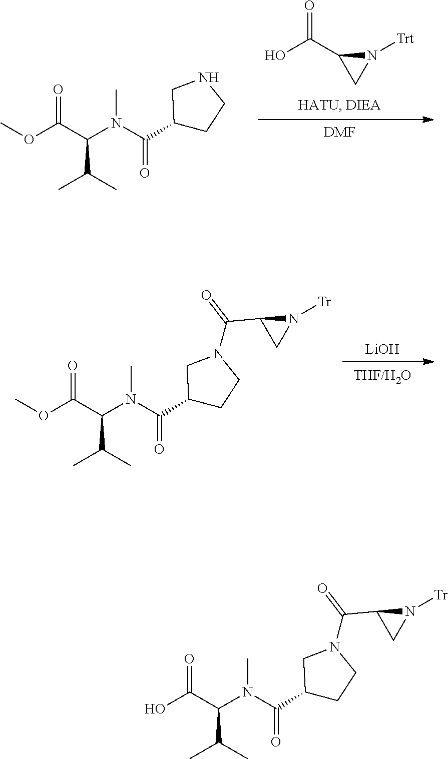

### Step 1: Synthesis of methyl N-methyl-N—((S)-1-((S)-1-tritylaziridine-2-carbonyl) pyrrolidine-3-carbonyl)-L-valinate

To a mixture of methyl N-methyl-N—((S)-pyrrolidine-3-carbonyl)-L-valinate (0.800 g, 3.30 mmol) and(S)-1-tritylaziridine-2-carboxylic acid (1.305 g, 3.96 mmol) in DMF (16 mL) at 0° C. was added DIPEA (2.9 mL, 16.5 mmol) and HATU (1.88 g, 4.9 mmol). The reaction mixture was warmed to room temperature and stirred for 1 h, at which point the mixture was diluted with EtOAc. The mixture was washed with sat. NH₄Cl and the resulting aqueous layer extracted with EtOAc. The combined organic layers were washed with brine, dried over Na₂SO₄, filtered, and concentrated under reduced pressure. The crude residue was purified by reverse phase chromatography (10→80% MeCN/H₂O) to afford the desired product (1.17 g, 64% yield) as a solid. LCMS (ESI) m/z: [M+H] calcd for C₃₄H₃₉N₃O₄: 554.30; found 554.3.

### Step 2: Synthesis of N-methyl-N—((S)-1-((S)-1-tritylaziridine-2-carbonyl) pyrrolidine-3-carbonyl)-L-valine

To a stirred solution of methyl N-methyl-N—((S)-1-((S)-1-tritylaziridine-2-carbonyl) pyrrolidine-3-carbonyl)-L-valinate (1.10 g, 1.99 mmol) in THF (10.0 mL) at 0° C. was added a 1M solution of LiOH (9.93 mL, 9.93 mmol). The reaction mixture was warmed to room temperature and stirred for 16 h. The reaction mixture was cooled to 0° C. and quenched with sat. aq. NH₄Cl to pH 6. The resulting mixture was extracted with EtOAc. The combined organic layers were washed with brine, dried over Na₂SO₄, filtered, and concentrated under reduced pressure to afford the desired crude product (1.2 g). LCMS (ESI) m/z: [M+H] calcd for C₃₃H₃₇N₃O₄: 540.29; found 540.3.

Intermediate A-3. Synthesis of N-methyl-N-(1-((R)-1-tritylaziridine-2-carbonyl) piperidine-4-carbonyl)-L-valine

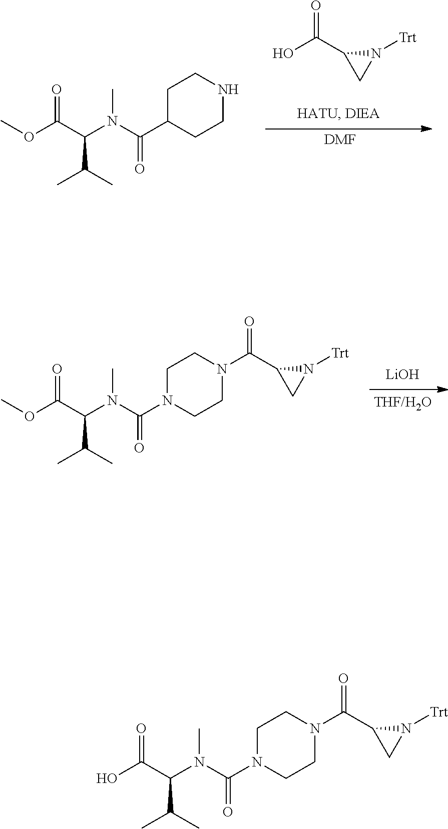

### Step 1: Synthesis of methyl N-methyl-N-(1-((R)-1-tritylaziridine-2-carbonyl) piperidine-4-carbonyl)-L-valinate

To a solution of (R)-1-tritylaziridine-2-carboxylic acid (1.157 g, 3.51 mmol) and methyl NV-methyl-N-(piperidine-4-carbonyl)-L-valinate (0.600 g, 2.34 mmol) in DMF (20 mL) at 0° C. was added DIPEA (0.204 mL, 11.70 mmol) and HATU (1.780 g, 4.68 mmol. After 3 h, the reaction mixture was extracted with EtOAc (200 mL). The combined organic layers were washed with brine (3×50 mL), dried over Na₂SO₄, filtered, and concentrated under reduced pressure. The residue was purified by reverse phase chromatography (10→50% MeCN/H₂O) to afford the desired product (740 mg, 55.7% yield) as a solid. LCMS (ESI) m/z: [M+H] calcd for C₃₅H₄₁N₃O₄: 568.32; found 568.3.

### Step 2: Synthesis of N-methyl-N-(1-((R)-1-tritylaziridine-2-carbonyl) piperidine-4-carbonyl)-L-valine

To a solution of methyl N-methyl-N-(1-((R)-1-tritylaziridine-2-carbonyl) piperidine-4-carbonyl)-L-valinate (0.700 g, 1.23 mmol) in THF (7.0 mL) at 0° C. was added a solution of LIOH·H₂O (0.259 g, 6.17 mmol) in H₂O (6.0 mL). The resulting solution was warmed to room temperature and stirred for 3 h. The reaction mixture was diluted with EtOAc (100 mL) and was washed with sat. brine (5×50 mL). The organic layer was dried over Na₂SO₄, filtered, and concentrated under reduced pressure to afford the crude product (700 mg) as a solid. LCMS (ESI) m/z: [M−H] calcd for C₃₄H₃₉N₃O₄: 552.29; found 552.2.

Intermediate A-4. Synthesis of N-methyl-N-(1-((S)-1-tritylaziridine-2-carbonyl) piperidine-4-carbonyl)-L-valine.

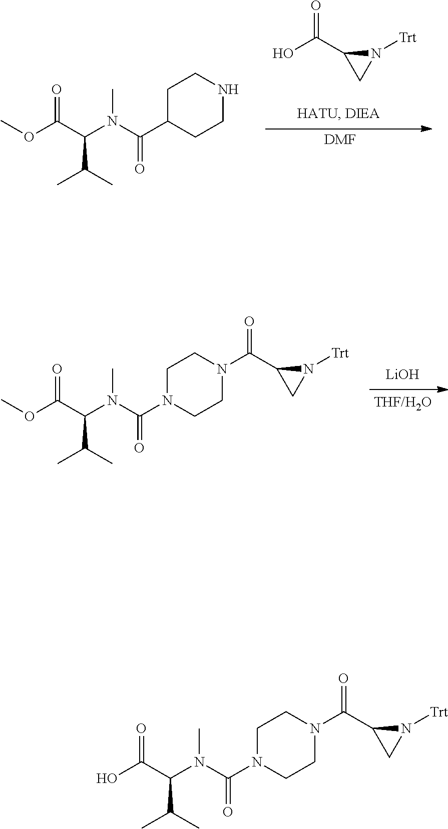

### Step 1: Synthesis of methyl N-methyl-N-(1-((S)-1-tritylaziridine-2-carbonyl) piperidine-4-carbonyl)-L-valinate

To a solution of methyl N-methyl-N-(piperidine-4-carbonyl)-L-valinate (0.550 g, 2.15 mmol) and(S)-1-tritylaziridine-2-carboxylic acid (0.848 g, 2.57 mmol) in DMF (10.0 mL) at 0° C. was added DIPEA (1.9 mL, 10.7 mmol) and HATU (1.2 g, 3.2 mmol). The reaction mixture was warmed to room temperature and stirred for 1 h. The reaction mixture was diluted with EtOAc (50 mL) and washed with sat. NH₄Cl (60 mL). The aqueous layer was extracted with EtOAc (3×50 mL). The combined organic layers were washed with brine (200 mL), dried over Na₂SO₄, filtered, and concentrated under reduced pressure. The residue was purified by reverse phase chromatography (10→80% MeCN/H₂O) to afford the desired product (1.2 g, 98.5% yield) as a solid. LCMS (ESI) m/z: [M+H] calcd for C₃₅H₄₁N₃O₄: 568.32; found 568.3.

### Step 2: Synthesis of N-methyl-N-(1-((S)-1-tritylaziridine-2-carbonyl) piperidine-4-carbonyl)-L-valine

To a solution of methyl N-methyl-N-(1-((S)-1-tritylaziridine-2-carbonyl) piperidine-4-carbonyl)-L-valinate (1.20 g, 2.11 mmol) in THF (11.0 mL) at 0° C. was added 1M LiOH (10.57 mL, 10.57 mmol). The resulting solution was warmed to room temperature and stirred for 16 h. The reaction mixture was cooled to 0° C. and quenched with sat. NH₄Cl until pH 6. The resulting mixture was extracted with EtOAc (3×50 mL) and the combined organic layers were washed with brine (3×50 mL), dried over Na₂SO₄, filtered, and concentrated under reduced pressure to afford the crude product (900 mg). LCMS (ESI) m/z: [M−H] calcd for C₃₄H₃₉N₃O₄: 554.29; found 554.3.

Intermediate A-5. Synthesis of N-methyl-N-(1-((R)-1-tritylaziridine-2-carbonyl) azetidine-3-carbonyl)-L-valine

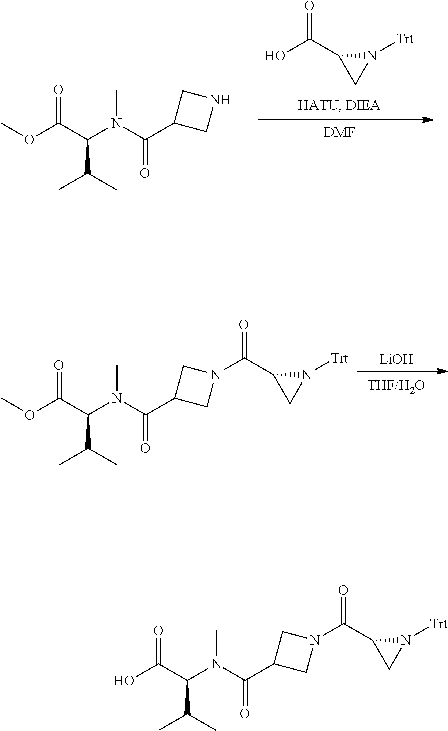

### Step 1: Synthesis of methyl N-methyl-N-(1-((R)-1-tritylaziridine-2-carbonyl) azetidine-3-carbonyl)-L-valinate

To a solution of methyl N-(azetidine-3-carbonyl)-N-methyl-L-valinate (0.410 g, 1.79 mmol) and (R)-1-tritylaziridine-2-carboxylic acid (0.887 g, 2.69 mmol) in DMF (10 mL) at 0° C. was added DIPEA (1.56 mL, 8.98 mmol) and HATU (1.37 g, 3.59 mmol). The reaction mixture was stirred for 1 h. The resulting mixture was then extracted with EtOAc (3×50 mL) and the combined organic layers were washed with brine (3×20 mL), dried over Na₂SO₄, filtered, and concentrated under reduced pressure. The residue was purified by reverse phase chromatography (10→80% MeCN/H₂O) to afford the desired product (650 mg, 67% yield) as a solid. LCMS (ESI) m/z: [M+H] calcd for C₃₃H₃₇N₃O₄: 540.29; found 540.3.

### Step 2: Synthesis of N-methyl-N-(1-((R)-1-tritylaziridine-2-carbonyl) azetidine-3-carbonyl)-L-valine

To a solution of methyl N-methyl-N-(1-((R)-1-tritylaziridine-2-carbonyl) azetidine-3-carbonyl)-L-valinate (0.650 mg, 1.20 mmol) in THF (10 mL) at 0° C. was added a 1M solution of LiOH·H₂O (6.03 mL). The reaction mixture was stirred for 3 h. The resulting mixture was then quenched with sat. NH₄Cl until pH 7. The resulting mixture was extracted with EtOAc (3×20 mL) and the combined organic layers were washed with brine (3×20 mL), dried over Na₂SO₄, filtered, and concentrated under reduced pressure to afford the desired crude product (588 mg) as a solid. LCMS (ESI) m/z: [M−H] calcd for C₃₂H₃₅N₃O₄: 526.27; found 526.3.

Intermediate A-6. Synthesis of N-methyl-N-(1-((S)-1-tritylaziridine-2-carbonyl) azetidine-3-carbonyl)-L-valine

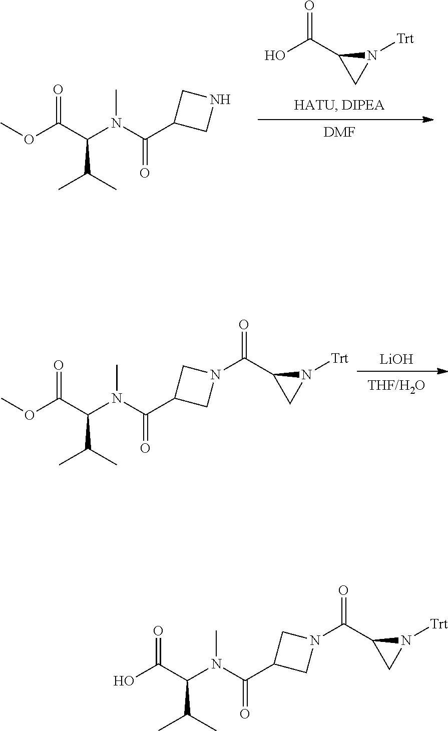

### Step 1: Synthesis of methyl N-methyl-N-(1-((S)-1-tritylaziridine-2-carbonyl) azetidine-3-carbonyl)-L-valinate

To a solution of methyl N-(azetidine-3-carbonyl)-N-methyl-L-valinate (0.550 g, 2.41 mmol) and(S)-1-tritylaziridine-2-carboxylic acid (0.952 g, 2.89 mmol) in DMF (10 mL) at 0° C. was added DIPEA (2.1 mL, 12.05 mmol) and HATU (1.37 g, 3.61 mmol). The reaction mixture was warmed to room temperature and stirred for 1 h. The resulting mixture was diluted with EtOAc (50 mL) and washed with sat. NH₄Cl (60 mL). The aqueous layer was then extracted with EtOAc (3×50 mL) and the combined organic layers were washed with brine (200 mL), dried over Na₂SO₄, filtered, and concentrated under reduced pressure. The residue was purified by reverse phase chromatography (10→80% MeCN/H₂O) to afford the desired product (820 mg, 63% yield) as a solid. LCMS (ESI) m/z: [M+H] calcd for C₃₃H₃₇N₃O₄: 540.29; found 540.3.

### Step 2: Synthesis of N-methyl-N-(1-((S)-1-tritylaziridine-2-carbonyl) azetidine-3-carbonyl)-L-valine

To a solution of methyl N-methyl-N-(1-((S)-1-tritylaziridine-2-carbonyl) azetidine-3-carbonyl)-L-valinate (0.800 g, 1.48 mmol) in THF (8.0 mL) at 0° C. was added 1M LiOH (7.41 mL, 7.41 mmol). The reaction mixture was warmed to room temperature and stirred for 16 h and was then cooled to 0° C. and quenched with sat. NH₄Cl until pH 6. The resulting mixture was extracted with EtOAc (3×50 mL) and the combined organic layers were washed with brine (150 mL), dried over Na₂SO₄, filtered, and concentrated under reduced pressure. The residue was purified by reverse phase chromatography (10→80% MeCN/H₂O+0.5% NH₄HCO₃) to afford the desired product (440 mg, 56% yield) as a solid. LCMS (ESI) m/z: [M−H] calcd for C₃₂H₃₅N₃O₄: 524.25; found 524.2.

Intermediate A-7. Synthesis of (2R,3S)-3-phenylaziridine-2-carboxylic acid

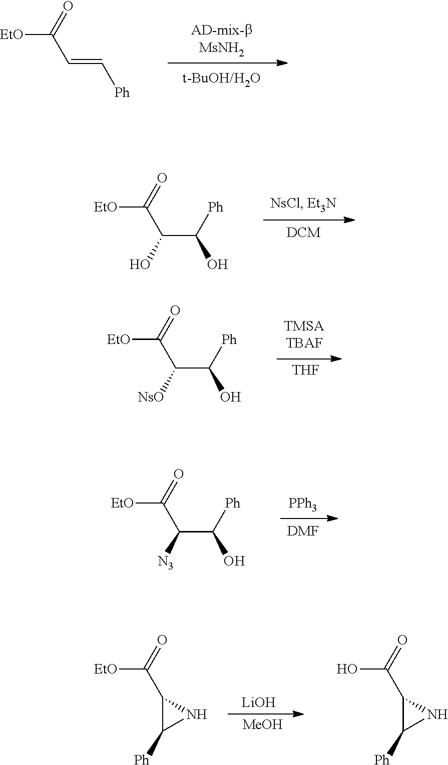

### Step 1: Synthesis of ethyl (2S,3R)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-β (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×90 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2S,3R)-3-hydroxy-2-(((4-nitrophenyl) sulfonyl)oxy)-3-phenylpropanoate

To a solution of ethyl (2S,3R)-2,3-dihydroxy-3-phenylpropanoate (2.0 g, 9.5 mmol) and Et₃N (3.97 mL, 28.5 mmol) in DCM (30.0 mL) at 0° C. was added 4-nitrobenzenesulfonyl chloride (2.11 g, 9.51 mmol). The resulting mixture was stirred for 1 h and was then diluted with H₂O (300 mL). The mixture was extracted with DCM (3×100 mL) and the combined organic layers were washed with brine (2×100 mL), dried over Na₂SO₄, filtered, and concentrated under reduced pressure. The residue was purified by prep-TLC(50% EtOAc/pet. ether) to afford the desired product (2.8 g, 67% yield) as a solid.

### Step 3: Synthesis of ethyl (2R,3R)-2-azido-3-hydroxy-3-phenylpropanoate

To a solution of ethyl (2S,3R)-3-hydroxy-2-(((4-nitrophenyl) sulfonyl)oxy)-3-phenylpropanoate (2.80 g, 7.08 mmol) in THF (30 mL) at room temperature was added trimethylsilyl azide (1.63 g, 14.2 mmol) and TBAF (1M in THF, 14.16 mL, 14.16 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50% EtOAc/pet. ether) to afford the desired product (1.2 g, 64% yield) as an oil.

### Step 4: Synthesis of ethyl (2R,3S)-3-phenylaziridine-2-carboxylate

To a solution of ethyl (2R,3R)-2-azido-3-hydroxy-3-phenylpropanoate (1.20 g, 5.10 mmol) in DMF (15.0 mL) was added PPh₃ (1.61 g, 6.12 mmol). The reaction mixture was stirred at room temperature for 30 min and then heated to 80° C. for an additional 16 h. The reaction mixture was then cooled to room temperature, diluted with H₂O (100 mL), and extracted with EtOAc (3×40 mL). The combined organic layers were washed with brine (20 mL), dried over Na₂SO, filtered, and concentrated under reduced pressure. The residue was purified by normal phase chromatography (16% EtOAc/pet. ether) to afford the desired product (620 mg, 57% yield) as an oil. LCMS (ESI) m/z: [M+H] calcd for C₁₁H₁₃NO_{2: 192.10}; found 192.0.

### Step 5: Synthesis of (2R,3S)-3-phenylaziridine-2-carboxylic acid

To a solution of ethyl (2R,3S)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL).

The reaction mixture was stirred for 1 h. The mixture was then diluted with MeCN (10 mL), and the resulting precipitate was collected by filtration and washed with MeCN (2×10 mL) to afford the crude desired product (70 mg) as a solid. LCMS (ESI) m/z: [M+H] calcd for C₉H₉NO₂: 164.07; found 164.0.

Intermediate A-8. Synthesis of (2,3R)-3-phenylaziridine-2-carboxylic acid

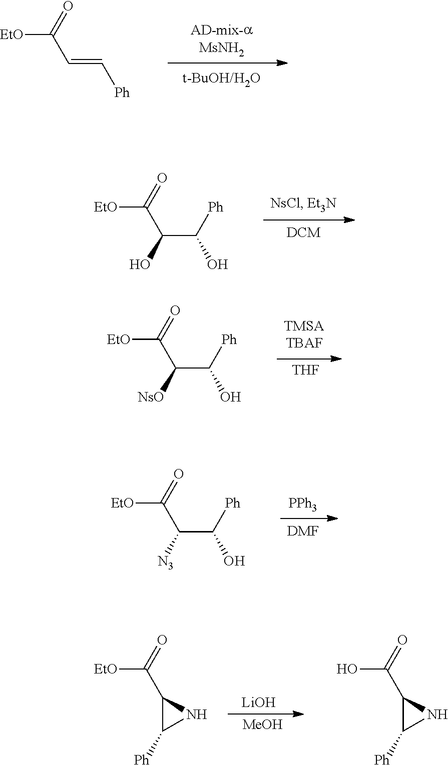

### Step 1: Synthesis of ethyl (2R,3S)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-α (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×80 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2R,3S)-3-hydroxy-2-(((4-nitrophenyl) sulfonyl)oxy)-3-phenylpropanoate

To a solution of ethyl (2R,3S)-2,3-dihydroxy-3-phenylpropanoate (2.10 g, 9.99 mmol) and Et₃N (4.18 mL, 29.9 mmol) in DCM (30.0 mL) at 0° C. was added 4-nitrobenzenesulfonyl chloride (2.21 g, 9.99 mmol). The resulting mixture was stirred for 1 h and was then diluted with H₂O (200 mL). The mixture was extracted with DCM (3×80 mL) and the combined organic layers were washed with brine (2×80 mL), dried over Na₂SO₄, filtered, and concentrated under reduced pressure. The residue was purified by prep-TLC(50% EtOAc/pet. ether) to afford the desired product (3.0 g, 68% yield) as a solid.

### Step 3: Synthesis of ethyl (2S,3S)-2-azido-3-hydroxy-3-phenylpropanoate

To a solution of ethyl (2R,3S)-3-hydroxy-2-(((4-nitrophenyl) sulfonyl)oxy)-3-phenylpropanoate (3.0 g, 7.59 mmol) in THF (30 mL) at room temperature was added trimethylsilyl azide (1.75 g, 15.2 mmol) and TBAF (1M in THF, 15.18 mL, 15.18 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50% EtOAc/pet. ether) to afford the desired product (1.4 g, 70% yield) as an oil.

### Step 4: Synthesis of ethyl (2S,3R)-3-phenylaziridine-2-carboxylate

To a solution of ethyl (2S,3S)-2-azido-3-hydroxy-3-phenylpropanoate (1.40 g, 5.95 mmol) in DMF (20.0 mL) was added PPh₃ (1.87 g, 7.14 mmol). The reaction mixture was stirred at room temperature for 30 min and then heated to 80° C. for an additional 16 h. The reaction mixture was then cooled to room temperature, diluted with H₂O (150 mL), and extracted with EtOAc (3×50 mL). The combined organic layers were washed with brine (40 mL), dried over Na₂SO, filtered, and concentrated under reduced pressure. The residue was purified by normal phase chromatography (16% EtOAc/pet. ether) to afford the desired product (720 mg, 56% yield) as an oil. LCMS (ESI) m/z: [M+H] calcd for C₁₁H₁₃NO_{2: 192.10}; found 192.0.

### Step 5: Synthesis of (2S,3R)-3-phenylaziridine-2-carboxylic acid

To a solution of ethyl (2S,3R)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL). The reaction mixture was stirred for 1 h. The mixture was then diluted with MeCN (10 mL), and the resulting precipitate was collected by filtration and washed with MeCN (2×10 mL) to afford the crude desired product (68 mg) as a solid. LCMS (ESI) m/z: [M+H] calcd for C₉H₉NO₂: 164.07; found 164.0.

Intermediate A-9. Synthesis of N—(N—((R)-1-benzylaziridine-2-carbonyl)-N-methylglycyl)-N-methyl-L-valine

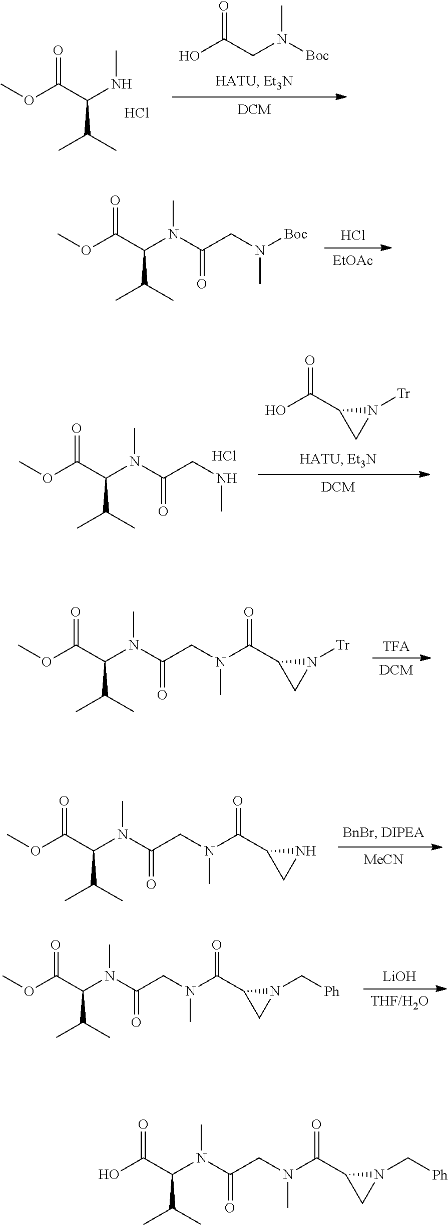

### Step 1: Synthesis of methyl N—(N-(tert-butoxycarbonyl)-N-methylglycyl)-N-methyl-L-valinate

To a solution of methyl methyl-L-valinate hydrochloride (4.0 g, 22.0 mmol) and N-(tert-butoxycarbonyl)-N-methylglycine (5.0 g, 26.4 mmol) in DCM (100.0 mL) was added Et₃N (9.2 mL, 66.1 mmol) and HATU (10.88 g, 28.63 mmol). The reaction mixture was stirred for 4 h. The reaction was then neutralized to pH 7 with sat. aq. NaHCO₃. The mixture was extracted with DCM and the combined organic layers were washed with brine, dried over Na₂SO₄, filtered, and concentrated under reduced pressure. The residue was purified by normal phase chromatography (50% EtOAc/pet. ether) to afford the desired product (6.2 g, 89% yield) as an oil. LCMS (ESI) m/z: [M+H] calcd for C₁₅H₂₈N₂O₅: 317.21; found 317.2.

### Step 2: Synthesis of methyl N-methyl-N-(methylglycyl)-L-valinate hydrochloride

To a solution of methyl N—(N-(tert-butoxycarbonyl)-N-methylglycyl)-N-methyl-L-valinate (4.97 g, 15.7 mmol) in EtOAc (150.0 mL) at 0° C. was added HCl (4M in dioxane, 50.0 mL, 200 mmol). The reaction mixture was stirred for 3 h and then concentrated under reduced pressure to afford the desired crude product (4.26 g, 107% yield) as an oil. LCMS (ESI) m/z: [M+H] calcd for C₁₀H₂₀N₂O₃: 217.16; found 217.1.

### Step 3: Synthesis of methyl N-methyl-N—(N-methyl-N—((R)-1-tritylaziridine-2-carbonyl) glycyl)-L-valinate

To a solution of methyl N-methyl-N-(methylglycyl)-L-valinate hydrochloride (1.0 g, 3.9 mmol) and (R)-1-tritylaziridine-2-carboxylic acid (1.30 g, 3.94 mmol) in DCM (25.0 mL) was added Et₃N (2.76 mL, 19.8 mmol) and HATU (1.81 g, 4.76 mmol). The reaction mixture was stirred for 1 h. The reaction was then neutralized to pH 7 with sat. aq. NaHCO₃. The mixture was extracted with DCM and the combined organic layers were washed with brine, dried over Na₂SO₄, filtered, and concentrated under reduced pressure. The residue was purified by normal phase chromatography (50% EtOAc/pet. ether) to afford the desired product (1.1 g, 52.6% yield) as a solid. LCMS (ESI) m/z: [M+H] calcd for C₃₂H₃₇N₃O₄: 528.29; found 528.2.

### Step 4: Synthesis of methyl N—(N—((R)-aziridine-2-carbonyl)-N-methylglycyl)-N-methyl-L-valinate

To a solution methyl N-methyl-N—(N-methyl-N—((R)-1-tritylaziridine-2-carbonyl) glycyl)-L-valinate (1.0 g, 3.9 mmol) in DCM (6 mL) at 0° C. was added TFA (2 mL). The reaction mixture was warmed to room temperature and stirred for 1 h, then concentrated under reduced pressure to afford the desired crude product (250 mg) as an oil. LCMS (ESI) m/z: [M+H] calcd for C₁₃H₂₃N₃O₄: 286.18; found 286.1.

### Step 5: Synthesis of methyl N—(N—((R)-1-benzylaziridine-2-carbonyl)-N-methylglycyl)-N-methyl-L-valinate

To a solution of methyl N—(N—((R)-aziridine-2-carbonyl)-N-methylglycyl)-N-methyl-L-valinate (220.0 mg, 0.771 mmol) in MeCN (2.0 mL) was added DIPEA (537 μL, 3.08 mmol) and benzyl bromide (101 μL, 0.848 mmol). The reaction mixture was stirred for 6 h. The reaction mixture was then concentrated under reduced pressure. The residue was purified by prep-TLC(9% MeOH/DCM) to afford the desired product (261 mg, 90% yield) as an oil. LCMS (ESI) m/z: [M+H] calcd for C₂₀H₂₉N₃O₄: 376.22; found 376.2.

### Step 6: Synthesis of N—(N—((R)-1-benzylaziridine-2-carbonyl)-N-methylglycyl)-N-methyl-L-valine

To a solution of methyl N—(N—((R)-1-benzylaziridine-2-carbonyl)-N-methylglycyl)-N-methyl-L-valinate (261.0 mg, 0.695 mmol) in THF (3.38 mL) was added a solution of LiOH (83.2 mg, 3.48 mmol) in H₂O (3.50 mL). The reaction mixture was stirred for 1 h. The reaction was then quenched with sat. aq. NH₄Cl. The resulting mixture was extracted with EtOAc and the combined organic layers were washed with brine, dried over Na₂SO₄, filtered, and concentrated under reduced pressure. The residue was purified by reverse phase chromatography (10→50% MeCN/H₂O) to afford the desired product (230 mg, 91% yield) as an oil. LCMS (ESI) m/z: [M+H] calcd for C₁₉H₂₇N₃O₄: 362.21; found 362.2.

Intermediate A-10. Synthesis of N—(N—((R)-1-benzylaziridine-2-carbonyl)-N-methylglycyl)-N-methyl-L-valine

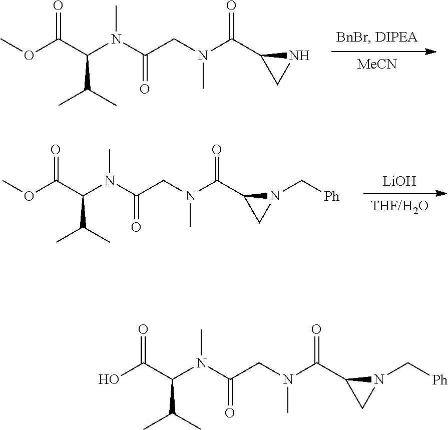

### Step 1: Synthesis of methyl N—(N—((S)-1-benzylaziridine-2-carbonyl)-N-methylglycyl)-N-methyl-L-valinate

To a solution of methyl N—(N—((S)-aziridine-2-carbonyl)-N-methylglycyl)-N-methyl-L-valinate (362.0 mg, 1.269 mmol) in MeCN (6.0 mL) at 0° C. was added DIPEA (883 μL, 5.08 mmol) and benzyl bromide (165 μL, 1.39 mmol). The reaction mixture was then warmed to room temperature and stirred overnight. The reaction mixture was then concentrated under reduced pressure. The residue was purified by prep-TLC(7% MeOH/DCM) to afford the desired product (287 mg, 60% yield) as an oil. LCMS (ESI) m/z: [M+H] calcd for C₂₀H₂₉N₃O₄: 376.22; found 376.2.

### Step 2: Synthesis of N—(N—((S)-1-benzylaziridine-2-carbonyl)-N-methylglycyl)-N-methyl-L-valine

To a solution of methyl N—(N—((S)-1-benzylaziridine-2-carbonyl)-N-methylglycyl)-N-methyl-L-valinate (270.0 mg, 0.719 mmol) in THF (3.6 mL) was added a solution of LiOH (86.1 mg, 3.59 mmol) in H₂O (3.60 mL). The reaction mixture was stirred for 30 min. The reaction was then quenched with sat. aq. NH₄Cl. The resulting mixture was extracted with EtOAc (3×15 mL) and the combined organic layers were washed with brine, dried over Na₂SO₄, filtered, and concentrated under reduced pressure to afford the desired crude product (240 mg, 92% yield) as an oil. LCMS (ESI) m/z: [M+H] calcd for C₁₉H₂₇N₃O₄: 362.21; found 362.2.

Intermediate A-11. Synthesis of N-methyl-N—(N-methyl-N—((R)-1-tritylaziridine-2-carbonyl) glycyl)-L-valine

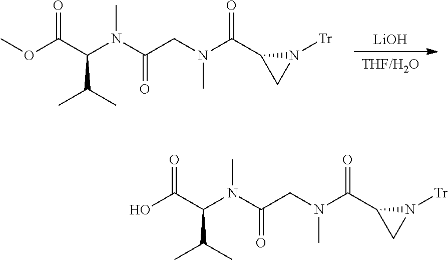

To a solution of methyl N-methyl-N—(N-methyl-N—((R)-1-tritylaziridine-2-carbonyl) glycyl)-L-valinate (1.30 g, 2.46 mmol) in THF (10.0 mL) at 0° C. was added a solution of LiOH (177.0 mg, 7.39 mmol) in H₂O (7.40 mL). The resulting mixture was warmed to room temperature, stirred for 3 h, and was then acidified to pH 5 with HCl (aq). The resulting mixture was extracted with EtOAc (3×80 mL) and the combined organic layers were washed with brine (2×50 mL), dried over Na₂SO₄, filtered, and concentrated under reduced pressure to afford the desired crude product (1 g, 71% yield). LCMS (ESI) m/z: [M+H] calcd for C₃₁H₃₅N₃O₄: 514.27; found 514.3.

Intermediate A-12. Synthesis of N-methyl-N-(4-((R)-1-tritylaziridine-2-carbonyl)-1,4-diazepane-1-carbonyl)-L-valine

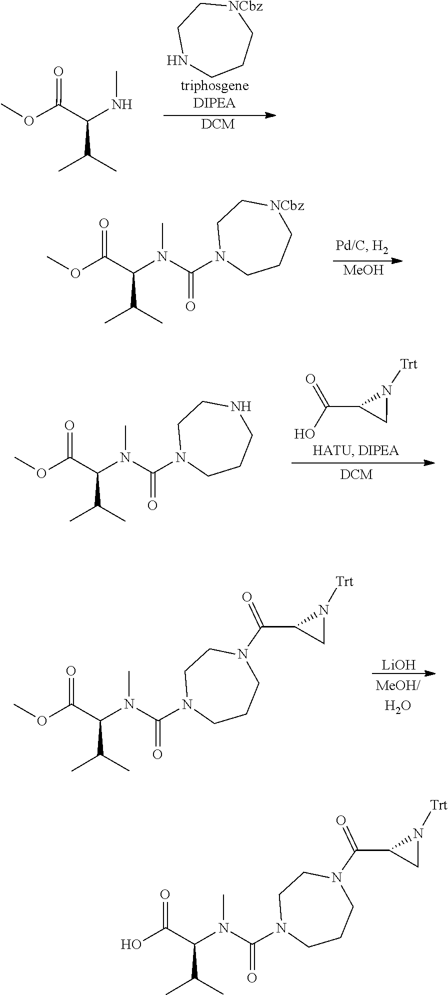

### Step 1: Synthesis of benzyl(S)-4-((1-methoxy-3-methyl-1-oxobutan-2-yl) (methyl) carbamoyl)-1,4-diazepane-1-carboxylate

To a solution of methyl N-methyl-L-valinate (2.50 g, 17.22 mmol) in DCM at 0° C. was added DIPEA (1.8 mL, 10.33 mmol) followed by triphosgene (2.55 g, 8.61 mmol). The resulting mixture was stirred for 3 h at 0° C. To the mixture was then added benzyl 1,4-diazepane-1-carboxylate (4.03 g, 17.20 mmol). The resulting mixture was warmed to room temperature and stirred overnight. The reaction was cooled to 0° C. and was quenched with NaHCO₃. The aqueous layer was extracted with EtOAc (2×30 mL) and the combined organic layers were washed with brine (2×30 mL), dried over Na₂SO₄, filtered, and concentrated under reduced pressure. The residue was purified by normal phase chromatography (25% EtOAc/pet. ether) to afford the desired product (3.5 g, 50.1% yield). LCMS (ESI) m/z: [M+H] calcd for C₂₁H₃₁N₃O₅: 406.23; found 406.5.

### Step 2: Synthesis of methyl N-(1,4-diazepane-1-carbonyl)-N-methyl-L-valinate

To a solution of benzyl(S)-4-((1-methoxy-3-methyl-1-oxobutan-2-yl) (methyl) carbamoyl)-1,4-diazepane-1-carboxylate (2.0 g, 4.93 mmol) in MeOH (20 mL) was added Pd/C (10% wt, 1 g). The mixture was placed under a hydrogen atmosphere (1 atm) and stirred for 2 h. The reaction mixture was filtered through a Celite and concentrated under reduced pressure to afford the desired crude product (1.3 g, 97.1% yield). LCMS (ESI) m/z: [M+H] calcd for C₁₃H₂₅N₃O₃: 272.20; found 272.3.

### Step 3: Synthesis of methyl N-methyl-N-(4-((R)-1-tritylaziridine-2-carbonyl)-1,4-diazepane-1-carbonyl)-L-valinate

To a solution of methyl N-(1,4-diazepane-1-carbonyl)-N-methyl-L-valinate (1.0 g, 3.69 mmol) and (R)-1-tritylaziridine-2-carboxylic acid (1.46 g, 4.42 mmol) in DMF at 0° C. was added DIPEA (1.93 mL, 11.06 mmol) followed by HATU (2.10 g, 5.52 mmol). The resulting mixture was warmed to room temperature and stirred for 1 h. The reaction mixture was then diluted with H₂O (15 mL) and the aqueous layer was extracted with EtOAc (3×30 mL). The combined organic layers were washed with brine (3×30 mL), dried over Na₂SO₄, filtered, and concentrated under reduced pressure. The residue was purified by normal phase chromatography (25% EtOAc/pet. ether) to afford the desired product (1.6 g, 74.5% yield). LCMS (ESI) m/z: [M+H] calcd for C₃₅H₄₂N₄O₄: 583.33; found 583.5.

### Step 4: Synthesis of N-methyl-N-(4-((R)-1-tritylaziridine-2-carbonyl)-1,4-diazepane-1-carbonyl)-L-valine

To a solution of methyl N-methyl-N-(4-((R)-1-tritylaziridine-2-carbonyl)-1,4-diazepane-1-carbonyl)-L-valinate (1.60 g, 2.75 mmol) in MeOH (10.0 mL) and H₂O (5.0 mL) at 0° C. was added LiOH (0.66 g, 27.56 mmol). The resulting mixture was warmed to room temperature and stirred overnight. The reaction mixture was acidified to pH 5 with HCl (aq) and the aqueous layer was extracted with EtOAc (3×30 mL). The combined organic layers were washed with brine (3×30 mL), dried over Na₂SO₄, filtered, concentrated under reduced pressure to afford the desired crude product (1.4 g, 95.6% yield). LCMS (ESI) m/z: [M+H] calcd for C₃₄H₄₀N₄O₄: 569.31; found 569.5.

Intermediate A-13. Synthesis of N-methyl-N-(4-((S)-1-tritylaziridine-2-carbonyl)-1,4-diazepane-1-carbonyl)-L-valine

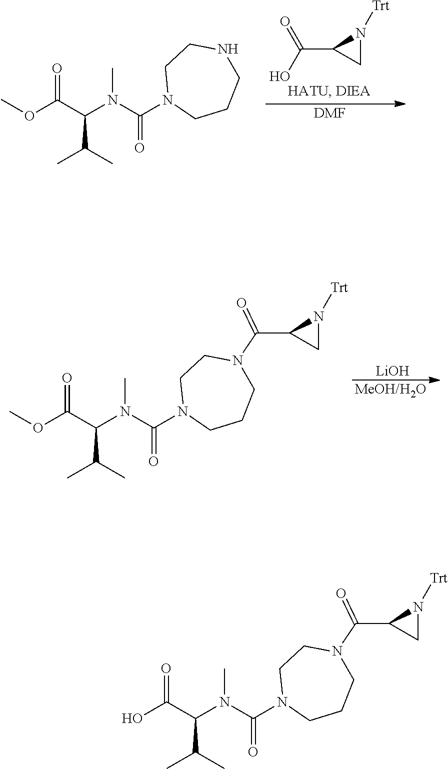

### Step 1: Synthesis of methyl N-methyl-N-(4-((S)-1-tritylaziridine-2-carbonyl)-1,4-diazepane-1-carbonyl)-L-valinate

To a solution of methyl N-(1,4-diazepane-1-carbonyl)-N-methyl-L-valinate (1.16 g, 4.28 mmol) and(S)-1-tritylaziridine-2-carboxylic acid (1.69 g, 5.13 mmol) in DMF (10 mL) at 0° C. was added DIPEA (2.23 mL, 12.82 mmol) followed by HATU (2.44 g, 6.41 mmol). The resulting mixture was stirred for 1 h at 0° C. The reaction mixture was then diluted with H₂O (15 mL) and the aqueous layer was extracted with EtOAc (3×15 mL). The combined organic layers were washed with brine (3×15 mL), dried over Na₂SO₄, filtered, and concentrated under reduced pressure. The residue was purified by normal phase chromatography (17% EtOAc/pet. ether) to afford the desired product (2 g, 80.3% yield). LCMS (ESI) m/z: [M+H] calcd for C₃₅H₄₂N₄O₄: 583.33; found 583.5.

### Step 2: Synthesis of N-methyl-N-(4-((S)-1-tritylaziridine-2-carbonyl)-1,4-diazepane-1-carbonyl)-L-valine

To a solution of methyl N-methyl-N-(4-((S)-1-tritylaziridine-2-carbonyl)-1,4-diazepane-1-carbonyl)-L-valinate (1.0 g, 1.72 mmol) in MeOH (8.0 mL) and H₂O (4.0 mL) at 0° C. was added LiOH (411 mg, 17.16 mmol). The resulting mixture was warmed to room temperature and stirred overnight. The reaction mixture was acidified to pH 5 with HCl (aq) and the aqueous layer was extracted with EtOAc (3×10 mL). The combined organic layers were washed with brine (3×10 mL), dried over Na₂SO₄, filtered, concentrated under reduced pressure to afford the desired crude product (0.6 g, 61.5% yield). LCMS (ESI) m/z: [M+H] calcd for C₃₄H₄₀N₄O₄: 569.31; found 569.5.

Intermediate A-14. Synthesis of N-methyl-N-(5-((S)-1-tritylaziridine-2-carboxamido) picolinoyl)-L-valine

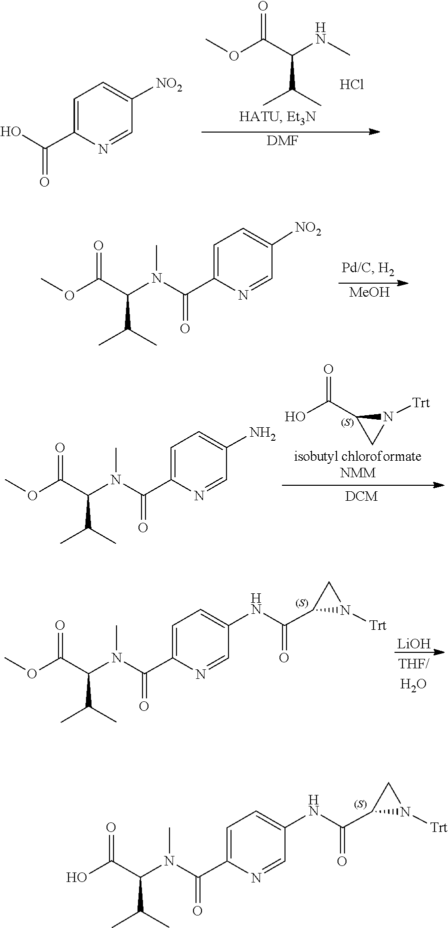

### Step 1: Synthesis of methyl N-methyl-N-(5-nitropicolinoyl)-L-valinate

To a solution of methyl N-methyl-L-valinate hydrochloride (190.0 mg, 1.31 mmol) and 5-nitropicolinic acid (200.0 mg, 1.19 mmol) in DMF (2 mL) at 0° C. was added HATU (678.6 mg, 1.79 mmol) and Et₃N (0.332 mL, 2.38 mmol). The resulting mixture was warmed to room temperature and stirred for 2 h. The resulting mixture was then extracted with EtOAc (2×50 mL) and the combined organic layers were washed with H₂O (20 mL) and brine (20 mL), dried over Na₂SO₄, filtered, and concentrated under reduced pressure. The residue was purified by normal phase chromatography (33% EtOAc/pet. ether) to afford the desired product (210 mg, 59.8% yield). LCMS (ESI) m/z: [M+H] calcd for C₁₃H₁₇N₃O₅: 296.12; found 296.0.

### Step 2: Synthesis of methyl N-(5-aminopicolinoyl)-N-methyl-L-valinate

To a solution of methyl N-methyl-N-(5-nitropicolinoyl)-L-valinate (5.0 g, 16.93 mmol) in MeOH (50.0 mL) was added Pd/C (2.50 g). The reaction mixture was placed under a hydrogen atmosphere (1 atm) and was stirred for 2 h. The mixture was filtered, the filter cake was washed with MeOH (2×20 mL), and the filtrate was concentrated under reduced pressure to afford the desired crude product (5.3 g). LCMS (ESI) m/z: [M+H] calcd for C₁₃H₁₉N₃O₃: 266.15; found 266.0.

### Step 3: Synthesis of methyl N-methyl-N-(5-((S)-1-tritylaziridine-2-carboxamido) picolinoyl)-L-valinate

To a solution(S)-1-tritylaziridine-2-carboxylic acid (55.9 mg, 0.17 mmol) in DCM at 0° C. was added isobutyl chloroformate (21.7 μL, 0.23 mmol) and N-methylmorpholine (66.8 μL, 0.61 mmol). The resulting mixture was stirred for 1 h and then methyl N-(5-aminopicolinoyl)-N-methyl-L-valinate (30.0 mg, 0.11 mmol) was added. The resulting mixture was warmed to room temperature and stirred for an additional 5 h. The mixture was extracted with DCM (3×50 mL) and the combined organic layers were washed with sat. NaHCO₃ (30 mL) and brine, dried over Na₂SO₄, filtered, and concentrated under reduced pressure. The residue was purified by normal phase chromatography (33% EtOAc/pet. ether) to afford the desired product (1.09 g, 66.9% yield). LCMS (ESI) m/z: [M+H] calcd for C₃₅H₃₆N₄O₄: 577.28; found 577.1.

### Step 4: Synthesis of N-methyl-N-(5-((S)-1-tritylaziridine-2-carboxamido) picolinoyl)-L-valine

To a solution methyl N-methyl-N-(5-((S)-1-tritylaziridine-2-carboxamido) picolinoyl)-L-valinate (100.0 mg, 0.17 mmol) in THF (0.5 mL) at 0° C. was added a solution of LiOH (20.76 mg, 0.87 mmol) in H₂O (0.5 mL). The resulting mixture was warmed to room temperature and stirred for 6 h. The mixture was acidified to pH 5 with 1 M citric acid. The resulting mixture was extracted with EtOAc (3×20 mL) and the combined organic layers were washed with brine (5 mL), dried over Na₂SO₄, filtered, and concentrated under reduced pressure to afford the desired crude product (76.8 mg, 78.7% yield). LCMS (ESI) m/z: [M+H] calcd for C₃₄H₃₄N₄O₄: 563.27; found 563.3.

Intermediate A-15. Synthesis of N-methyl-N-(5-((R)-1-tritylaziridine-2-carboxamido) picolinoyl)-L-valine

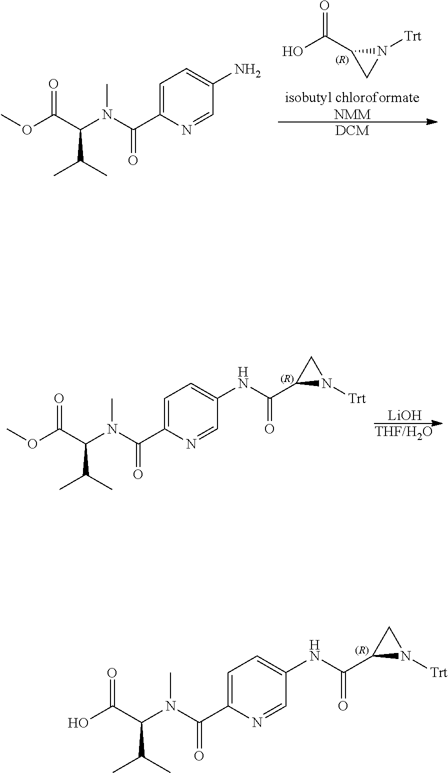

### Step 1: Synthesis of methyl N-methyl-N-(5-((R)-1-tritylaziridine-2-carboxamido) picolinoyl)-L-valinate

To a solution (R)-1-tritylaziridine-2-carboxylic acid (1396.7 mg, 4.24 mmol) in DCM (8 mL) at 0° C. was added isobutyl chloroformate (440 μL, 3.39 mmol) and N-methylmorpholine (466 μL, 4.24 mmol). The resulting mixture was stirred for 1 h and then methyl N-(5-aminopicolinoyl)-N-methyl-L-valinate (750.0 mg, 2.83 mmol) was added. The resulting mixture was warmed to room temperature and stirred for an additional 5 h. The mixture was quenched by the addition of NaHCO₃ and the aqueous layer was extracted with DCM (2×100 mL). The combined organic layers were washed with brine (120 mL), dried over Na₂SO₄, filtered, and concentrated under reduced pressure. The residue was purified by normal phase chromatography (50% EtOAc/pet. ether) to afford the desired product (580 mg, 35.6% yield). LCMS (ESI) m/z: [M+H] calcd for C₃₅H₃₆N₄O₄: 577.28; found 577.2.

### Step 2: Synthesis of N-methyl-N-(5-((R)-1-tritylaziridine-2-carboxamido) picolinoyl)-L-valine

To a solution methyl N-methyl-N-(5-((R)-1-tritylaziridine-2-carboxamido) picolinoyl)-L-valinate (558.0 mg, 0.97 mmol) in THF (14 mL) at 0° C. was added a solution of LiOH (115.9 mg, 4.84 mmol) in H₂O (14 mL). The resulting mixture was warmed to room temperature and stirred for 6 h. The mixture was acidified to pH 5 with 1 M citric acid. The resulting mixture was extracted with EtOAc (3×100 mL) and the combined organic layers were washed with brine (30 mL), dried over Na₂SO₄, filtered, and concentrated under reduced pressure to afford the desired crude product (580 mg, 78.7% yield). LCMS (ESI) m/z: [M+H] calcd for C₃₄H₃₄N₄O₄: 563.27; found 563.2.

Intermediate A-16. Synthesis of (2R,3S)-1-((R)-tert-butylsulfinyl)-3-(methoxycarbonyl) aziridine-2-carboxylic acid

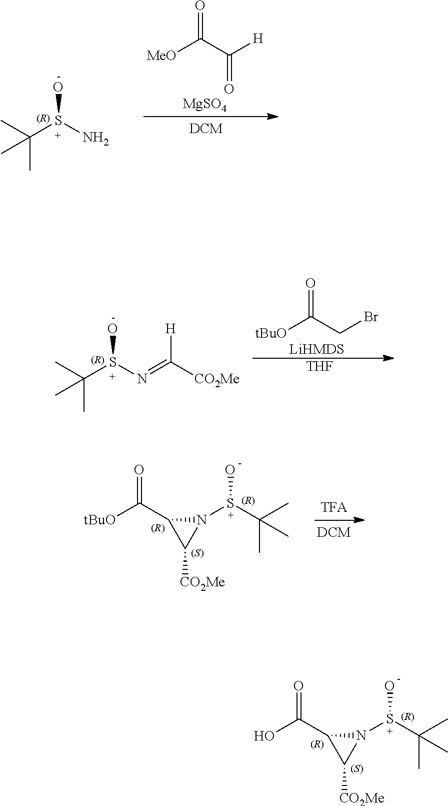

### Step 1: Synthesis of methyl (R,E)-2-((tert-butylsulfinyl) imino) acetate

To a solution of (R)-2-methylpropane-2-sulfinamide (13.21 g, 109.01 mmol) and methyl 2-oxoacetate (8.0 g, 90.85 mmol) in DCM (130 mL) at room temperature was added MgSO4 (54.67 g, 454.23 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.8 g, 33.4% yield). LCMS (ESI) m/z: [M+H] calcd for C₇H₁₃NO₃S: 192.07; found 191.9.

### Step 2: Synthesis of 2-(tert-butyl) 3-methyl (2R,3S)-1-((R)-tert-butylsulfinyl) aziridine-2,3-dicarboxylate

To a solution of 1M LiHMDS (61.40 mL, 61.40 mmol) in THF (300.0 mL) at −78° C. was added tert-butyl 2-bromoacetate (11.83 g, 60.65 mmol). The resulting mixture was stirred for 30 min. To the reaction mixture was then added methyl methyl (R,E)-2-((tert-butylsulfinyl) imino) acetate (5.8 g, 30.33 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.34 g, 4.5% yield). LCMS (ESI) m/z: [M+H] calcd for C₁₃H₂₃NO₅S: 306.14; found 306.2.

### Step 3: Synthesis of (2R,3S)-1-((R)-tert-butylsulfinyl)-3-(methoxycarbonyl) aziridine-2-carboxylic acid

To a solution of 2-(tert-butyl) 3-methyl (2R,3S)-1-((R)-tert-butylsulfinyl) aziridine-2,3-dicarboxylate (302.0 mg, 0.99 mmol) in DCM (3.0 mL) at 0° C. was added TFA (1.50 mL). The resulting mixture was stirred for 1 h and then concentrated under reduced pressure to afford the desired crude product (300 mg). LCMS (ESI) m/z: [M+H] calcd for C₉H₁₅NO₅S: 250.07; found 250.1.

Intermediate A-17. Synthesis of (2R,3S)-1-((S)-tert-butylsulfinyl)-3-(methoxycarbonyl) aziridine-2-carboxylic acid

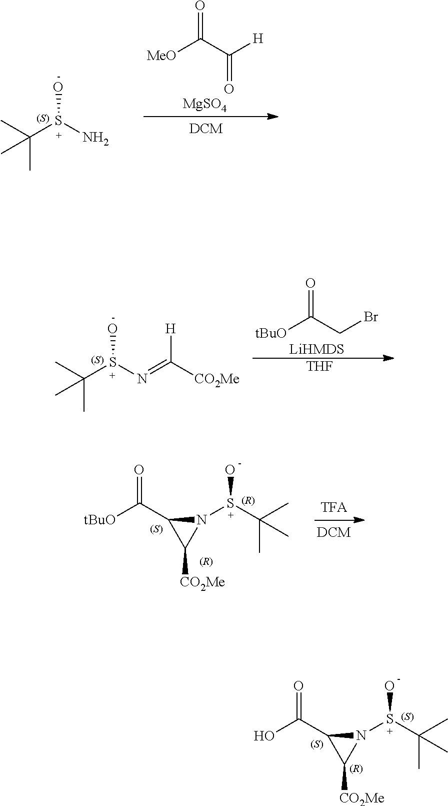

### Step 1: Synthesis of methyl (S,E)-2-((tert-butylsulfinyl) imino) acetate

To a solution of(S)-2-methylpropane-2-sulfinamide (9.81 g, 80.94 mmol) and methyl 2-oxoacetate (5.94 g, 67.45 mmol) in DCM (100 mL) at room temperature was added MgSO4 (40.60 g, 337.26 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.68 g, 44.0% yield). LCMS (ESI) m/z: [M+H] calcd for C₇H₁₃NO₃S: 192.07; found 191.1.

### Step 2: Synthesis of 2-(tert-butyl) 3-methyl (2R,3S)-1-((S)-tert-butylsulfinyl) aziridine-2,3-dicarboxylate

To a solution of 1M LiHMDS (59.40 mL, 59.40 mmol) in THF (300.0 mL) at −78° C. was added tert-butyl 2-bromoacetate (11.59 g, 59.40 mmol). The resulting mixture was stirred for 30 min. To the reaction mixture was then added methyl methyl (S,E)-2-((tert-butylsulfinyl) imino) acetate (5.68 g, 29.70 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.26 g, 13.9% yield). LCMS (ESI) m/z: [M+H] calcd for C₁₃H₂₃NO₅S: 306.14; found 306.1.

### Step 3: Synthesis of (2R,3S)-1-((S)-tert-butylsulfinyl)-3-(methoxycarbonyl) aziridine-2-carboxylic acid

To a solution of 2-(tert-butyl) 3-methyl (2R,3S)-1-((S)-tert-butylsulfinyl) aziridine-2,3-dicarboxylate (457.0 mg, 1.50 mmol) in DCM (6.0 mL) at 0° C. was added TFA (3.0 mL). The resulting mixture was stirred for 1 h and then concentrated under reduced pressure to afford the desired crude product (450 mg). LCMS (ESI) m/z: [M+H] calcd for C₉H₁₅NO₅S: 250.07; found 250.1.

Intermediate A-18. Synthesis of (2R,3R)-1-((R)-tert-butylsulfinyl)-3-cyclopropylaziridine-2-carboxylic acid

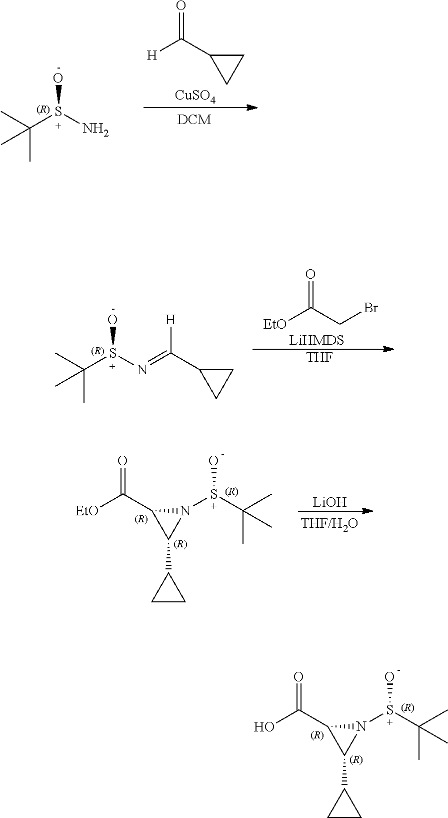

### Step 1: Synthesis of (R,E)-N-(cyclopropylmethylene)-2-methylpropane-2-sulfinamide

To a solution of (R)-2-methylpropane-2-sulfinamide (1.0 g, 8.25 mmol) and cyclopropanecarbaldehyde (1.16 g, 16.55 mmol) in DCM (50 mL) at room temperature was added CuSO₄ (3.95 g, 24.75 mmol). The resulting mixture was stirred overnight. The reaction mixture was then filtered, the filter cake washed with EtOAc, and the filtrate was concentrated under reduced pressure. The residue was purified by prep-TLC(17% EtOAc/pet. ether) to afford the desired product (1.4 g, 97.9% yield). LCMS (ESI) m/z: [M+H] calcd for C₈H₁₅NOS: 174.10; found 174.1.

### Step 2: Synthesis of ethyl (2R,3R)-1-((R)-tert-butylsulfinyl)-3-cyclopropylaziridine-2-carboxylate

To a solution of 1M LiHMDS (23 mL, 23 mmol) in THF (50.0 mL) at −78° C. was added ethyl bromoacetate (3.83 g, 22.95 mmol). The resulting mixture was warmed to −70° C. and stirred for 1 h. To the reaction mixture was then added (R,E)-N-(cyclopropylmethylene)-2-methylpropane-2-sulfinamide (2.0 g, 11.48 mmol). The resulting mixture was stirred for 1 h at −70° C. The reaction mixture was warmed to 0° C. and quenched with H₂O. The aqueous layer was extracted with EtOAc (3×100 mL). The combined organic layers were washed with brine, dried over Na₂SO₄, filtered, and concentrated under reduced pressure. The residue was purified by prep-TLC(25% EtOAc/pet. ether) to afford the desired product (1.8 g, 60.5% yield). LCMS (ESI) m/z: [M+H] calcd for C₁₂H₂₁NO₃S: 306.14; found 260.13.

### Step 3: Synthesis of (2R,3R)-1-((R)-tert-butylsulfinyl)-3-cyclopropylaziridine-2-carboxylic acid

To a solution of ethyl (2R,3R)-1-((R)-tert-butylsulfinyl)-3-cyclopropylaziridine-2-carboxylate (900.0 mg, 3.47 mmol) in THF (3.0 mL) and H₂O (3.0 mL) at 0° C. was added LiOH·H₂O (218.4 mg, 5.21 mmol). The resulting mixture was stirred for 1 h and was then quenched by H₂O. The aqueous layer was extracted with EtOAc (3×50) and the combined organic layers were washed with brine, dried over Na₂SO₄, filtered, and concentrated under reduced pressure to afford the desired crude product (400 mg, 29.9% yield). LCMS (ESI) m/z: [M+H] calcd for C₁₀H₁₇NO₃S: 232.10; found 232.1.

Intermediate A-19. Synthesis of (2R,3R)-1-((R)-tert-butylsulfinyl)-3-methylaziridine-2-carboxylic acid

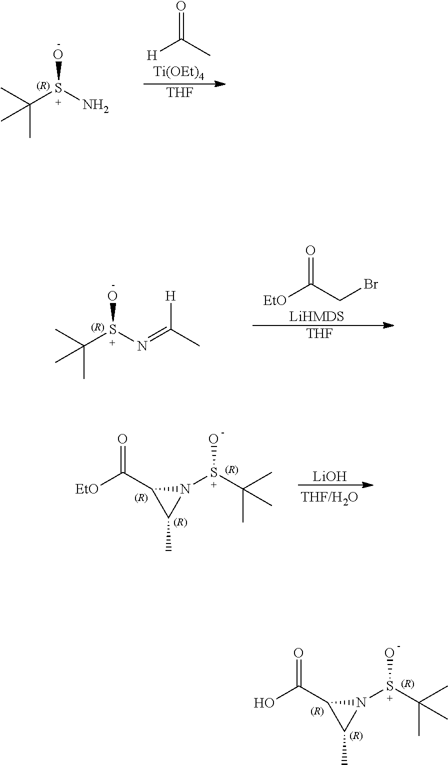

### Step 1: Synthesis of (R,E)-N-ethylidene-2-methylpropane-2-sulfinamide

To a solution of (R)-2-methylpropane-2-sulfinamide (3.0 g, 24.75 mmol) and tetraethoxytitanium (1.7 g, 7.43 mmol) in THF (30 mL) at 0° C. was added acetaldehyde (218.1 mg, 4.95 mmol). The resulting mixture was stirred for 20 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure. Purification by normal phase chromatography (9% EtOAc/pet. ether) afforded desired product (3 g, 82% yield). LCMS (ESI) m/z: [M+H] calcd for C₆H₁₃NOS: 148.08; found 148.0.

### Step 2: Synthesis of ethyl (2R,3R)-1-((R)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (R,E)-N-ethylidene-2-methylpropane-2-sulfinamide (3.0 g, 20.38 mmol). The resulting mixture was stirred for 2 h at −78° C. and then quenched with H₂O (300 mL). The aqueous layer was extracted with EtOAc (3×300 mL) and the combined organic layers were washed with brine (3×100 mL), dried over Na₂SO₄, filtered, and concentrated under reduced pressure. The residue was purified by reverse phase chromatography (10→50% MeCN/H₂O) to afford the desired product (1.4 g, 29.5% yield). LCMS (ESI) m/z: [M+H] calcd for C₁₀H₁₉NO₃S: 234.12; found 234.1.

### Step 3: Synthesis of (2R,3R)-1-((R)-tert-butylsulfinyl)-3-methylaziridine-2-carboxylic acid

To a solution of ethyl (2R,3R)-1-((R)-tert-butylsulfinyl)-3-methylaziridine-2-carboxylate (1.0 g 4.29 mmol) in THF (6.4 mL) and H₂O (6.4 mL) at 0° C. was added LiOH·H₂O (539.5 mg, 12.86 mmol). The resulting mixture was warmed to room temperature and stirred for 2 h and was then neutralized to pH 5 with HCl (aq.) and sat. NH₄Cl (aq.). The aqueous layer was extracted with EtOAc (3×10 mL) and the combined organic layers were washed with brine, dried over Na₂SO₄, filtered, and concentrated under reduced pressure to afford the desired crude product (489 mg, 55.6% yield). LCMS (ESI) m/z: [M+H] calcd for C₈H₁₅NO₃S: 206.09; found 206.0.

### Intermediate A-20. Synthesis of (2S,3S)-1-(S)-tert-butylsulfinyl)-3-methylaziridine-2-carboxylic acid

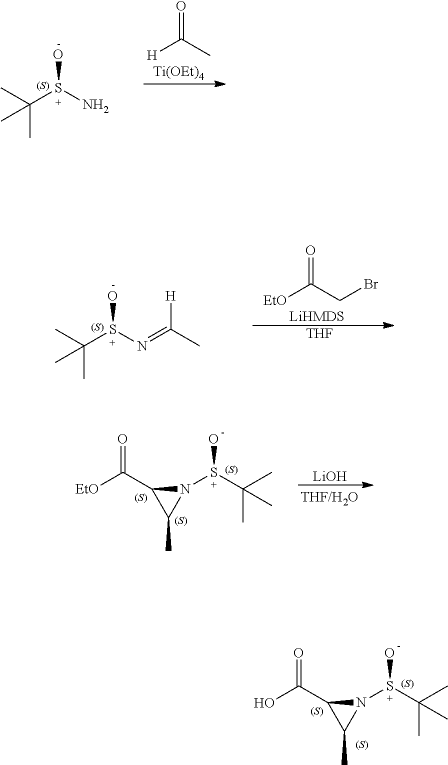

### Step 1: Synthesis of (S,E)-N-ethylidene-2-methylpropane-2-sulfinamide

To a mixture of(S)-2-methylpropane-2-sulfinamide (5.0 g, 41.25 mmol) and tetraethoxytitanium (18.82 g, 82.51 mmol) at 0° C. was added acetaldehyde (3.63 g, 82.51 mmol). The resulting mixture was warmed to room temperature and stirred for 30 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure to afford desired crude product (3.9 g, 64% yield). LCMS (ESI) m/z: [M+H] calcd for C₆H₁₃NOS: 148.08; found 148.2.

### Step 2: Synthesis of ethyl (2S,3S)-1-((S)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (S,E)-N-ethylidene-2-methylpropane-2-sulfinamide (3.0 g, 20.38 mmol). The resulting mixture was stirred for 2 h at −78° C. and then quenched with H₂O. The aqueous layer was extracted with EtOAc (3×200 mL) and the combined organic layers were washed with brine (3×300 mL), dried over Na₂SO₄, filtered, and concentrated under reduced pressure. The residue was purified by reverse phase chromatography (10→50% MeCN/H₂O) to afford the desired product (2 g, 42% yield). LCMS (ESI) m/z: [M+H] calcd for C₁₀H₁₉NO₃S: 234.12;

found 234.0.

### Step 3: Synthesis of (2S,3S)-1-((S)-tert-butylsulfinyl)-3-methylaziridine-2-carboxylic acid

To a solution of ethyl (2S,3S)-1-((S)-tert-butylsulfinyl)-3-methylaziridine-2-carboxylate (80.0 mg, 0.34 mmol) in THF (1.0 mL) and H₂O (0.2 mL) at 0° C. was added LiOH·H₂O (32.9 mg, 1.37 mmol). The resulting mixture was warmed to room temperature and stirred for 4 h and was then acidified to pH 3 with HCl (aq.). The aqueous layer was extracted with EtOAc (3×10 mL) and the combined organic layers were washed with brine, dried over Na₂SO₄, filtered, and concentrated under reduced pressure to afford the desired crude product (70 mg, 99% yield). LCMS (ESI) m/z: [M+H] calcd for C₈H₁₅NO₃S: 206.09; found 206.0.

### Intermediate A-21 and A-22. Synthesis of N-methyl-N—((S)-1-(((R)-1-methylaziridin-2-yl) sulfonyl) pyrrolidine-3-carbonyl)-L-valine and N-methyl-N—((S)-1-(((S)-1-methylaziridin-2-yl) sulfonyl) pyrrolidine-3-carbonyl)-L-valine

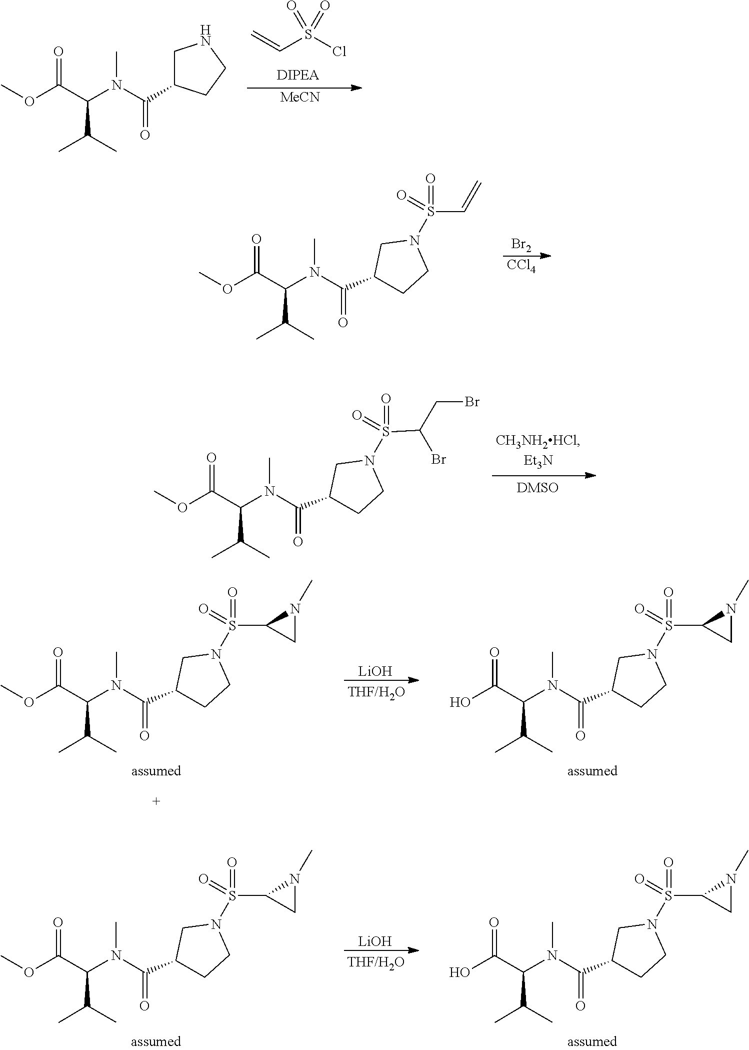

### Step 1: Synthesis of methyl N-methyl-N—((S)-1-(vinylsulfonyl) pyrrolidine-3-carbonyl)-L-valinate

To a mixture of methyl N-methyl-N—((S)-pyrrolidine-3-carbonyl)-L-valinate (7.0 g, 28.89 mmol) in MeCN (200 mL) at −20° C. was added DIPEA (10.0 mL, 57.78 mmol) followed by ethenesulfonyl chloride (4.0 g, 31.78 mmol). The resulting solution was stirred for 2 h at −20° C. and was then diluted with EtOAc (800 mL). The resulting solution was washed with brine (3×100 mL) and concentrated under reduced pressure. Purification by normal phase chromatography (50% EtOAc/pet. ether) afforded the desired product (4.8 g, 49.9%, yield). LCMS (ESI) m/z: [M+H] calcd for C₁₄H₂₄N₂O₅S: 333.15; found 333.1.

### Step 2: Synthesis of methyl N-((3S)-1-((1,2-dibromoethyl) sulfonyl) pyrrolidine-3-carbonyl)-N-methyl-L-valinate

To a solution of methyl N-methyl-N—((S)-1-(vinylsulfonyl) pyrrolidine-3-carbonyl)-L-valinate (4.5 g, 13.54 mmol) in CCl₄ (100 mL) at 0° C. was added Br₂ (2.77 mL, 54.15 mmol). The resulting solution was stirred for overnight and was then quenched by the addition of sat. NaHCO₃ (100 mL). The aqueous layer was extracted with EtOAc (3×200 mL) and the combined organic layers were washed with brine, dried with Na₂SO₄, filtered, and concentrated under reduced pressure. Purification by normal phase chromatography (25% EtOAc/pet. ether) afforded the desired product (2.6 g, 39.0% yield). LCMS (ESI) m/z: [M+H] calcd for C₁₄H₂₄Br₂N₂O₅S: 492.99; found 493.0.

### Step 3: Synthesis of methyl N-methyl-N—((S)-1-(((R)-1-methylaziridin-2-yl) sulfonyl) pyrrolidine-3-carbonyl)-L-valinate and methyl N-methyl-N—((S)-1-(((S)-1-methylaziridin-2-yl) sulfonyl) pyrrolidine-3-carbonyl)-L-valinate

To a solution of methyl N-((3S)-1-((1,2-dibromoethyl) sulfonyl) pyrrolidine-3-carbonyl)-N-methyl-L-valinate (2.6 g, 5.28 mmol) in DMSO (250 mL) was added methanamine hydrochloride (1.07 g, 15.85 mmol) and Et₃N (7.37 mL, 52.82 mmol). The reaction mixture was heated to 75° C. and stirred overnight. The mixture was then cooled to room temperature and diluted with EtOAc (1.5 L). The resulting mixture was washed with sat. NH₄Cl (2×200 mL) and brine (2×200 mL) and the organic layer was then concentrated under reduced pressure. Purification by reverse phase chromatography (40→60% MeCN/H₂O) afforded a mixture of the desired products. The diastereomers were separated by prep-SFC(28% MeOH/CO₂) to afford methyl N-methyl-N—((S)-1-(((R)-1-methylaziridin-2-yl) sulfonyl) pyrrolidine-3-carbonyl)-L-valinate (0.46 g, 24% yield) and methyl N-methyl-N—((S)-1-(((S)-1-methylaziridin-2-yl) sulfonyl) pyrrolidine-3-carbonyl)-L-valinate (0.35 g, 18.3% yield). LCMS (ESI) m/z: [M+H] calcd for C₁₅H₂₇N₃O₅S: 362.17; found 362.1.

### Step 4: Synthesis of N-methyl-N—((S)-1-(((R)-1-methylaziridin-2-yl) sulfonyl) pyrrolidine-3-carbonyl)-L-valine

To a solution of methyl N-methyl-N—((S)-1-(((R)-1-methylaziridin-2-yl) sulfonyl) pyrrolidine-3-carbonyl)-L-valinate (200.0 mg, 0.55 mmol) in THF (2.0 mL) and H₂O (2.0 mL) at 0° C. was added LiOH (53.0 mg, 2.21 mmol). The resulting solution was stirred for 2 h at 0° C. and then the reaction mixture was acidified to pH 6 with 1M HCl. The aqueous layer was extracted with EtOAc and the combined organic layers were washed with brine, dried with Na₂SO₄, filtered, and concentrated under reduced pressure. Purification by reverse phase chromatography (5→55% MeCN/H₂O) afforded the desired product (110 mg, 57.2%, yield). LCMS (ESI) m/z: [M+H] calcd for C₁₄H₂₅N₃O₅S: 348.16; found 348.1.

### Step 5: Synthesis of N-methyl-N—((S)-1-(((S)-1-methylaziridin-2-yl) sulfonyl) pyrrolidine-3-carbonyl)-L-valine

To a solution of methyl N-methyl-N—((S)-1-(((S)-1-methylaziridin-2-yl) sulfonyl) pyrrolidine-3-carbonyl)-L-valinate (200.0 mg, 0.55 mmol) in THF (2.0 mL) and H₂O (2.0 mL) at 0° C. was added LiOH (53.0 mg, 2.21 mmol). The resulting solution was stirred for 2 h at 0° C. and then the reaction mixture was acidified to pH 6 with 1M HCl. The aqueous layer was extracted with EtOAc and the combined organic layers were washed with brine, dried with Na₂SO₄, filtered, and concentrated under reduced pressure. Purification by reverse phase chromatography (5→55 MeCN/H₂O) afforded the desired product (121 mg, 62.9%, yield). LCMS (ESI) m/z: [M+H] calcd for C₁₄H₂₅N₃O₅S: 348.16; found 348.1.

### Intermediate A-23. Synthesis of (2S)-3-methyl-2-(1-oxo-7-((S)-1-tritylaziridine-2-carbonyl)-2,7-diazaspiro[4.4]nonan-2-yl) butanoic acid

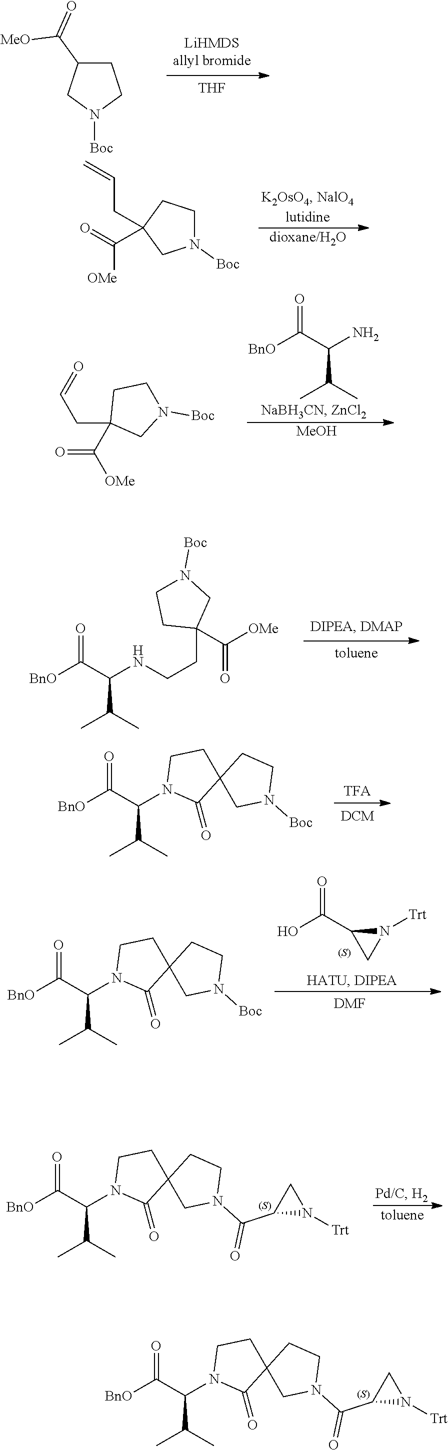

### Step 1: Synthesis of 1-(tert-butyl) 3-methyl 3-allylpyrrolidine-1,3-dicarboxylate

To a mixture of 1-(tert-butyl) 3-methyl pyrrolidine-1,3-dicarboxylate (10 g, 43.616 mmol) in THF (100 mL) at −78° C. was added 1M LiHMDS (65.42 mL, 65.424 mmol) dropwise. The resulting mixture was stirred at −78° C. for 1 h and then a solution of allyl bromide (7.91 g, 65.423 mmol) in THF was added dropwise over 10 min. The resulting mixture was stirred at −78° C. for an additional 2 h and was then quenched by the addition of sat. NH₄Cl at 0° C. The resulting mixture was extracted with EtOAc (3×100 mL) and the combined organic layers were washed with brine (2×80 mL), dried with Na₂SO₄, filtered, and concentrated under reduced pressure. Purification by silica gel column chromatography (20% EtOAc/pet. ether) afforded the desired product (10 g, 76% yield).

### Step 2: Synthesis of 1-(tert-butyl) 3-methyl 3-(2-oxoethyl) pyrrolidine-1,3-dicarboxylate

To a mixture of 1-(tert-butyl) 3-methyl 3-allylpyrrolidine-1,3-dicarboxylate (11.0 g, 40.84 mmol) and 2,6-lutidine (8.75 g, 81.68 mmol) in dioxane (190 mL) and H₂O (19 mL) at 0° C. was added K₂OsO₄·2H₂O (0.75 g, 2.04 mmol). The resulting mixture was stirred at 0° C. for 15 min and then NaIO₄ (34.94 g, 163.36 mmol) was added in portions. The mixture was warmed to room temperature and stirred for an additional 3 h, then was quenched by the addition of sat. Na₂S₂O₃ at 0° C. The resulting mixture was extracted with EtOAc (3×300 mL) and the combined organic layers were washed with brine (200 mL), dried with Na₂SO₄, filtered, and concentrated under reduced pressure. Purification by reverse phase chromatography (0→40% MeCN/H₂O, 0.1% HCO₂H) afforded the desired product (6.4 g, 51% yield).

### Step 3: Synthesis of 1-(tert-butyl) 3-methyl 3-(2-(((S)-1-(benzyloxy)-3-methyl-1-oxobutan-2-yl) amino) ethyl) pyrrolidine-1,3-dicarboxylate

To a mixture of 1-(tert-butyl) 3-methyl 3-(2-oxoethyl) pyrrolidine-1,3-dicarboxylate (6.30 g, 23.220 mmol) and benzyl L-valinate (7.22 g, 34.831 mmol) in MeOH (70 mL) at 0° C. was added ZnCl₂ (4.75 g, 34.831 mmol). The resulting mixture was warmed to room temperature and stirred for 30 min, then cooled to 0° C. NaBH₃CN (2.92 g, 46.441 mmol) was added in portions then the mixture was warmed to room temperature and stirred for 2 h. The reaction was quenched by the addition of sat. NH₄Cl at 0° C. and the resulting mixture was then extracted with EtOAc (3×200 mL). The combined organic layers were washed with brine (150 mL), dried with Na₂SO₄, filtered, and concentrated under reduced pressure. Purification by silica gel column chromatography (33% EtOAc/pet. ether) afforded the desired product (6.4 g, 53% yield). LCMS (ESI) m/z: [M+H] calcd for C₂₅H₃₈N₂O₆: 463.28; found 463.3.

### Step 4: Synthesis of tert-butyl 7-((S)-1-(benzyloxy)-3-methyl-1-oxobutan-2-yl)-6-oxo-2,7-diazaspiro[4.4]nonane-2-carboxylate

To a mixture of 1-(tert-butyl) 3-methyl 3-(2-(((S)-1-(benzyloxy)-3-methyl-1-oxobutan-2-yl) amino) ethyl) pyrrolidine-1,3-dicarboxylate (4.50 g, 9.728 mmol) and DIPEA (16.6 mL, 97.28 mmol) in toluene (50 mL) was added DMAP (1.19 g, 9.728 mmol) and then mixture was heated to 80° C. After 24 h the reaction was cooled to room temperature and concentrated under reduced pressure. Purification by reverse phase chromatography (15→60% MeCN/H₂O, 0.1% HCO₂H) afforded the desired product (3 g, 64% yield). LCMS (ESI) m/z: [M+H] calcd for C₂₄H₃₄N₂O₅: 431.26; found 431.2.

### Step 5: Synthesis of benzyl (2S)-3-methyl-2-(1-oxo-2,7-diazaspiro[4.4]nonan-2-yl) butanoate

To a solution of tert-butyl 7-((S)-1-(benzyloxy)-3-methyl-1-oxobutan-2-yl)-6-oxo-2,7-diazaspiro[4.4]nonane-2-carboxylate (400.0 mg, 0.929 mmol) in DCM (3.0 mL) at 0° C. was added TFA (1.50 mL, 20.195 mmol) dropwise. The resulting mixture was stirred at 0° C. for 1 h and was then concentrated under reduced pressure. The TFA residue was further removed by azeotropic distillation with toluene three times to afford the desired product (400 mg, crude). LCMS (ESI) m/z: [M+H] calcd for C₁₉H₂₆N₂O₃: 331.20; found 331.1.

### Step 6: Synthesis of benzyl (2S)-3-methyl-2-(1-oxo-7-((S)-1-tritylaziridine-2-carbonyl)-2,7-diazaspiro[4.4]nonan-2-yl) butanoate

To a solution of benzyl (2S)-3-methyl-2-(1-oxo-2,7-diazaspiro[4.4]nonan-2-yl) butanoate (400.0 mg, 1.21 mmol) and DIPEA (2.06 mL, 12.11 mmol) in DMF (5 mL) at 0° C. was added(S)-1-tritylaziridine-2-carboxylic acid (558.26 mg, 1.695 mmol) followed by COMU (673.55 mg, 1.574 mmol) in portions. The resulting mixture was stirred at 0° C. for 1 h and was then diluted with H₂O (50 mL). The aqueous layer was extracted with EtOAc (3×20 mL) and the combined organic layers were washed with brine (20 mL), dried with Na₂SO₄, filtered, and concentrated under reduced pressure. Purification by prep-TLC(33% EtOAc/pet. ether) afforded the desired product (510 mg, 59% yield). LCMS (ESI) m/z: [M+H] calcd for C₄₁H₄₃N₃O₄: 642.34; found 642.3.

### Step 7: Synthesis of (2S)-3-methyl-2-(1-oxo-7-((S)-1-tritylaziridine-2-carbonyl)-2,7-diazaspiro[4.4]nonan-2-yl) butanoic acid

To a mixture of benzyl (2S)-3-methyl-2-(1-oxo-7-((S)-1-tritylaziridine-2-carbonyl)-2,7-diazaspiro[4.4]nonan-2-yl) butanoate (480.0 mg, 0.748 mmol) in toluene (35.0 mL) was added Pd/C 200.0 mg, 1.879 mmol). The resulting mixture was placed under an atmosphere of H₂ (1 atm), heated to 50° C. and stirred for 3 h. The mixture was cooled to room temperature, filtered, the filter cake was washed with MeOH (3×10 mL), and the filtrate was concentrated under reduced pressure to afford the desired product (310 mg, 67% yield). LCMS (ESI) m/z: [M−H] calcd for C₃₄H₃₇N₃O₄: 550.27; found 550.3.

### Intermediate A-24. Synthesis of (2S)-3-methyl-2-(1-oxo-7-((R)-1-tritylaziridine-2-carbonyl)-2,7-diazaspiro[4.4]nonan-2-yl) butanoic acid

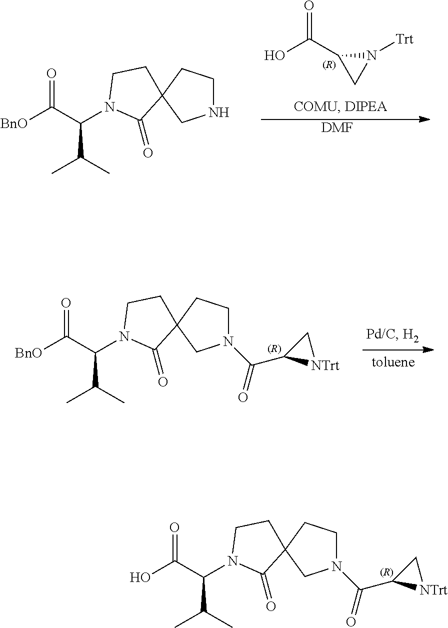

### Step 1: Synthesis of benzyl (2S)-3-methyl-2-(1-oxo-7-((R)-1-tritylaziridine-2-carbonyl)-2,7-diazaspiro[4.4]nonan-2-yl) butanoate

To a solution of benzyl (2S)-3-methyl-2-(1-oxo-2,7-diazaspiro[4.4]nonan-2-yl) butanoate (400.0 mg, 1.21 mmol) and (R)-1-tritylaziridine-2-carboxylic acid (518.4 mg, 1.57 mmol) in DMF (4.0 mL) at 0° C. was added DIPEA (1.0 mL, 6.05 mmol) followed by COMU (621.7 mg, 1.45 mmol). The resulting mixture was stirred for 1 h and was then diluted with H₂O (40 mL). The aqueous layer was extracted with EtOAc (3×15 mL) and the combined organic layers were washed with brine (2×10 mL), dried over Na₂SO₄, filtered and concentrated under reduced pressure. The residue was purified by prep-TLC(33% EtOAc/pet. ether) to afford the desired product (540 mg, 62% yield). LCMS (ESI) m/z: [M+H] calcd for C₄₁H₄₃N₃O₄: 642.33; found 642.4.

### Step 2: Synthesis of (2S)-3-methyl-2-(1-oxo-7-((R)-1-tritylaziridine-2-carbonyl)-2,7-diazaspiro[4.4]nonan-2-yl) butanoic acid

To a solution of benzyl (2S)-3-methyl-2-(1-oxo-7-((R)-1-tritylaziridine-2-carbonyl)-2,7-diazaspiro[4.4]nonan-2-yl) butanoate (510.0 mg, 0.80 mmol) in toluene (30 mL) was added Pd/C (250.0 mg, 2.35 mmol). The resulting mixture was placed under a hydrogen atmosphere (1 atm), heated to 50° C., and stirred for 3 h. The reaction was then cooled to room temperature, filtered, the filter cake was washed with MeOH (3×10 mL), and the filtrate was concentrated under reduced pressure to afford the desired crude product (330 mg). LCMS (ESI) m/z: [M+H] calcd for C₃₄H₃₇N₃O₄: 552.29; found 552.3.

### Intermediate A-25 and A-26. Synthesis of benzyl(S)-3-methyl-2-((S)-1-oxo-2,7-diazaspiro[4.4]nonan-2-yl) butanoate and benzyl(S)-3-methyl-2-((R)-1-oxo-2,7-diazaspiro[4.4]nonan-2-yl) butanoate

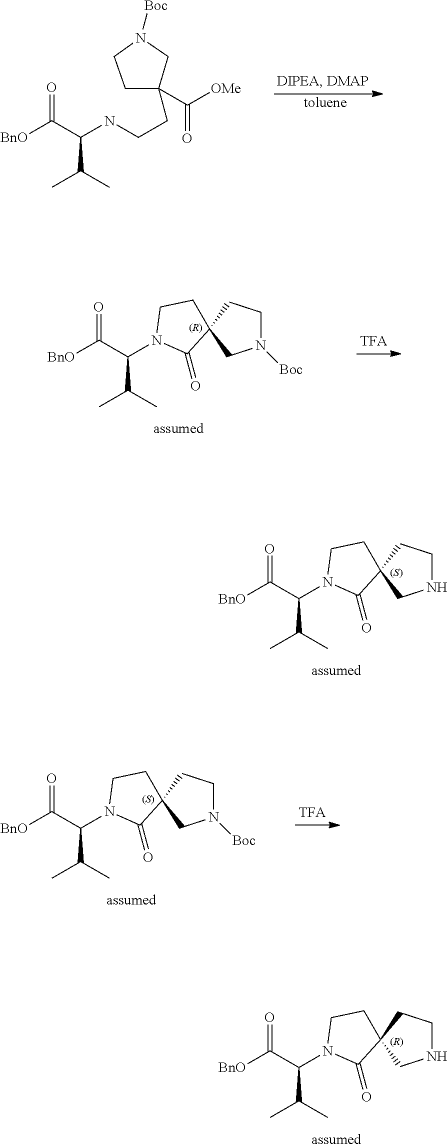

### Step 1: Synthesis of tert-butyl (R)-7-((S)-1-(benzyloxy)-3-methyl-1-oxobutan-2-yl)-6-oxo-2,7-diazaspiro[4.4]nonane-2-carboxylate and tert-butyl(S)-7-((S)-1-(benzyloxy)-3-methyl-1-oxobutan-2-yl)-6-oxo-2,7-diazaspiro[4.4]nonane-2-carboxylate

To a mixture of 1-(tert-butyl) 3-methyl 3-(2-(((S)-1-(benzyloxy)-3-methyl-1-oxobutan-2-yl) amino) ethyl) pyrrolidine-1,3-dicarboxylate (4.50 g, 9.728 mmol) and DIPEA (16.6 mL, 97.28 mmol) in toluene (50 mL) was added DMAP (1.19 g, 9.728 mmol) and then mixture was heated to 80° C. After 24 h the reaction was cooled to room temperature and concentrated under reduced pressure. Purification by reverse phase chromatography (10→50% MeCN/H₂O, 0.1% HCO₂H). The diastereomers were then separated by chiral prep-SFC(30% EtOH/CO₂) to afford tert-butyl (R)-7-((S)-1-(benzyloxy)-3-methyl-1-oxobutan-2-yl)-6-oxo-2,7-diazaspiro[4.4]nonane-2-carboxylate (1.0 g, 32% yield, LCMS (ESI) m/z: [M+H] calcd for C₂₄H₃₄N₂O₅: 431.26; found 431.2) and tert-butyl(S)-7-((S)-1-(benzyloxy)-3-methyl-1-oxobutan-2-yl)-6-oxo-2,7-diazaspiro[4.4]nonane-2-carboxylate carboxylate (1.0 g, 32% yield, LCMS (ESI) m/z: [M+H] calcd for C₂₄H₃₄N₂O₅: 431.26; found 431.2).

### Step 2: Synthesis of benzyl(S)-3-methyl-2-((S)-1-oxo-2,7-diazaspiro[4.4]nonan-2-yl) butanoate

To a solution of tert-butyl (5R)-7-[(2S)-1-(benzyloxy)-3-methyl-1-oxobutan-2-yl]-6-oxo-2,7-diazaspiro[4.4]nonane-2-carboxylate (1.40 g, 3.25 mmol) in DCM (14 mL) at 0° C. was added TFA (5.0 mL, 67.3 mmol). The resulting mixture was stirred at 0° C. for 1 h and was then concentrated under reduced pressure. The mixture was diluted with H₂O (20 mL) and was basified to pH 8 with sat. NaHCO₃(aq.) at 0° C. The resulting mixture was extracted with EtOAc (3×50 mL) and combined organic layers were washed with brine (40 mL), dried over Na₂SO₄, filtered, and concentrated under reduced pressure to afford the desired product (1.4 g, crude). LCMS (ESI) m/z: [M+H] calcd for C₁₉H₂₆N₂O₃: 331.20; found 331.2).

### Step 3: Synthesis of benzyl(S)-3-methyl-2-((R)-1-oxo-2,7-diazaspiro[4.4]nonan-2-yl) butanoate

To a solution of tert-butyl (5S)-7-[(2S)-1-(benzyloxy)-3-methyl-1-oxobutan-2-yl]-6-oxo-2,7-diazaspiro[4.4]nonane-2-carboxylate (1.0 g, 2.3 mmol) in DCM (10 mL) at 0° C. was added TFA (4.0 mL, 53.9 mmol). The resulting mixture was stirred at 0° C. for 1 h and was then concentrated under reduced pressure. The mixture was diluted with H₂O (10 mL) and was basified to pH 8 with sat. NaHCO₃(aq.) at 0° C. The resulting mixture was extracted with EtOAc (3×20 mL) and combined organic layers were washed with brine (20 mL), dried over Na₂SO₄, filtered, and concentrated under reduced pressure to afford the desired product (1.0 g, crude). LCMS (ESI) m/z: [M+H] calcd for C₁₉H₂₆N₂O₃: 331.20; found 331.1).

### Intermediate A-27. Synthesis of (2S)-3-methyl-2-(1-oxo-7-((R)-1-tritylaziridine-2-carbonyl)-2,7-diazaspiro[4.4]nonan-2-yl) butanoic acid

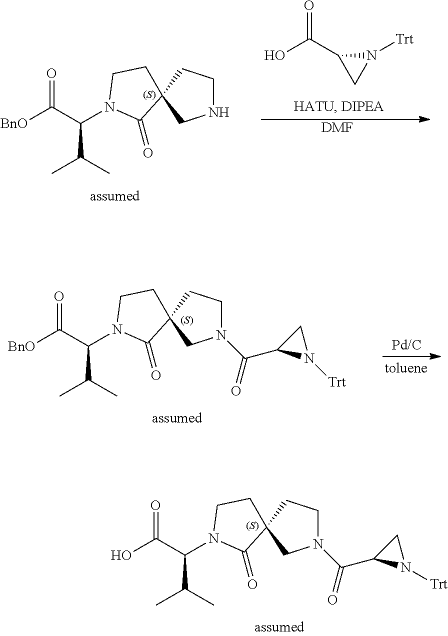

### Step 1: Synthesis of benzyl(S)-3-methyl-2-((S)-1-oxo-7-((R)-1-tritylaziridine-2-carbonyl)-2,7-diazaspiro[4.4]nonan-2-yl) butanoate

To a solution of benzyl(S)-3-methyl-2-((S)-1-oxo-2,7-diazaspiro[4.4]nonan-2-yl) butanoate (400 mg, 1.2 mmol) and DIPEA (1.1 mL, 6.1 mmol) in DMF (5.0 mL) at 0° C. was added (R)-1-tritylaziridine-2-carboxylic acid (480 mg, 1.5 mmol) and HATU (550 mg, 1.5 mmol). The resulting mixture was stirred for 1 h then purified by reverse phase chromatography (15→80% MeCN/H₂O, 0.5% NH₄HCO₃) to afford the desired product (500 mg, 57% yield). LCMS (ESI) m/z: [M+H] calcd for C₄₁H₄₃N₃O₄: 642.34; found 642.3.

### Step 2: Synthesis of (2S)-3-methyl-2-(1-oxo-7-((R)-1-tritylaziridine-2-carbonyl)-2,7-diazaspiro[4.4]nonan-2-yl) butanoic acid

A solution of benzyl(S)-3-methyl-2-((S)-1-oxo-7-((R)-1-tritylaziridine-2-carbonyl)-2,7-diazaspiro[4.4]nonan-2-yl) butanoate (450 mg, 0.70 mmol) and Pd/C (120 mg, 1.13 mmol) in toluene (30 mL) at 50° C. was stirred under a hydrogen atmosphere (1 atm). The mixture was stirred for 3 h and then was filtered, and the filter cake was washed with MeOH (3×30 mL). The filtrate was concentrated under reduced pressure to afford the desired product (430 mg, crude). LCMS (ESI) m/z: [M+H] calcd for C₃₄H₃₇N₃O₄: 552.29; found 552.3.

### Intermediate 28. Synthesis of(S)-3-methyl-2-((R)-1-oxo-7-((R)-1-tritylaziridine-2-carbonyl)-2,7-diazaspiro[4.4]nonan-2-yl) butanoic acid

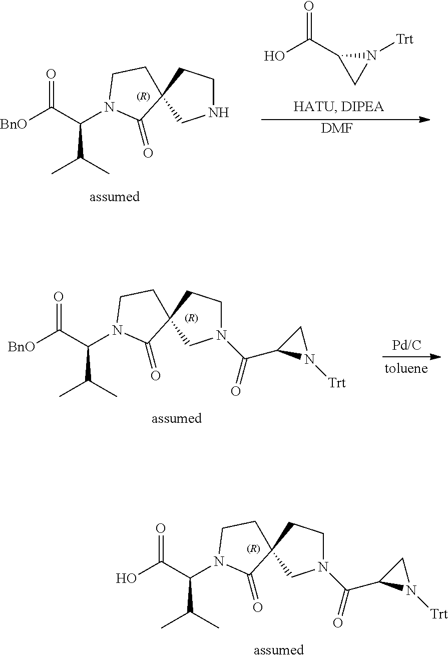

### Step 1: Synthesis of benzyl(S)-3-methyl-2-((R)-1-oxo-7-((R)-1-tritylaziridine-2-carbonyl)-2,7-diazaspiro[4.4]nonan-2-yl) butanoate

To a solution of benzyl(S)-3-methyl-2-((R)-1-oxo-2,7-diazaspiro[4.4]nonan-2-yl) butanoate (500 mg, 1.5 mmol) and DIPEA (1.3 mL, 7.6 mmol) in DMF (7.0 mL) at 0° C. was added (R)-1-tritylaziridine-2-carboxylic acid (550 mg, 1.7 mmol) and HATU (630 mg, 1.7 mmol). The resulting mixture was stirred for 1 h then purification by reverse phase chromatography (10→80% MeCN/H₂O, 0.5% NH₄HCO₃) afforded desired product (700 mg, 64% yield) as an off-white solid. LCMS (ESI) m/z: [M+H] calcd for C₄₁H₄₃N₃O₄: 642.34; found 642.3.

### Step 2: Synthesis of(S)-3-methyl-2-((R)-1-oxo-7-((R)-1-tritylaziridine-2-carbonyl)-2,7-diazaspiro[4.4]nonan-2-yl) butanoic acid

A solution of benzyl(S)-3-methyl-2-((R)-1-oxo-7-((R)-1-tritylaziridine-2-carbonyl)-2,7-diazaspiro[4.4]nonan-2-yl) butanoate (650 mg, 0.70 mmol) and Pd/C (140 mg, 1.3 mmol) in toluene (30 mL) at 50° C. was stirred under a hydrogen atmosphere (1 atm). The mixture was stirred for 3 h and then was filtered, and the filter cake was washed with MeOH (3×30 mL). The filtrate was concentrated under reduced pressure to afford the desired product (550 mg, crude). LCMS (ESI) m/z: [M+H] calcd for C₃₄H₃₇N₃O₄: 552.29; found 552.3.

### Intermediate A-29. Synthesis of(S)-2-((R)-7-(tert-butoxycarbonyl)-1-oxo-2,7-diazaspiro[4.4]nonan-2-yl)-3-methylbutanoic acid

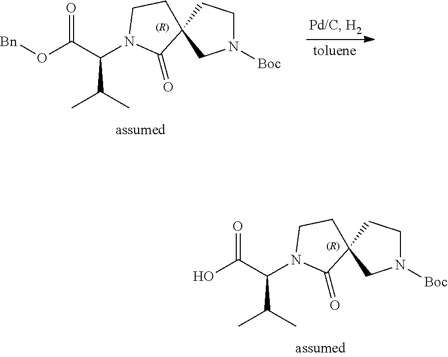

To a solution of tert-butyl (R)-7-((S)-1-(benzyloxy)-3-methyl-1-oxobutan-2-yl)-6-oxo-2,7-diazaspiro[4.4]nonane-2-carboxylate (600 mg, 1.4 mmol) in toluene (20 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z: [M−H] calcd for C₁₇H₂₈N₂O₅: 339.19; found 339.1.

### Intermediate A-30. Synthesis of(S)-2-((S)-7-(tert-butoxycarbonyl)-1-oxo-2,7-diazaspiro[4.4]nonan-2-yl)-3-methylbutanoic acid

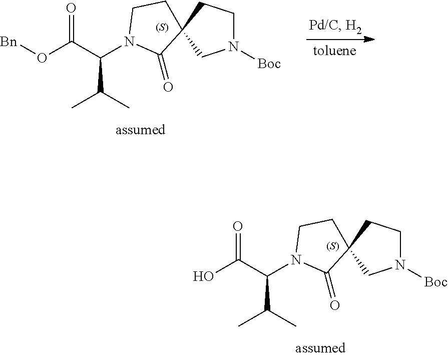

To a solution of tert-butyl(S)-7-((S)-1-(benzyloxy)-3-methyl-1-oxobutan-2-yl)-6-oxo-2,7-diazaspiro[4.4]nonane-2-carboxylate (550 mg, 1.3 mmol) in toluene (30 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z: [M−H] calcd for C₁₇H₂₈N₂O₅: 339.19; found 339.2.

### Intermediate A-31. Synthesis of (R)-3-methyl-2-(((S)—N-methyl-1-tritylaziridine-2-carboxamido) methyl) butanoic acid

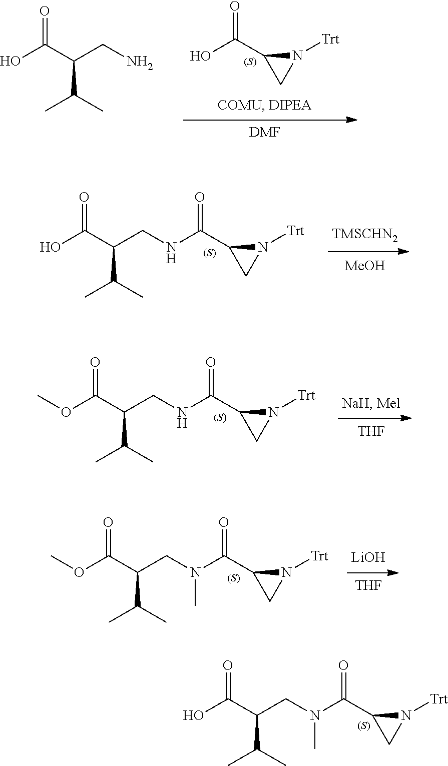

### Step 1: Synthesis of (R)-3-methyl-2-(((S)-1-tritylaziridine-2-carboxamido) methyl) butanoic acid

To a solution of(S)-1-tritylaziridine-2-carboxylic acid (1 g, 2.9 mmol) in DMF (10 mL) at 0° C. was added DIPEA (2.5 mL, 14.55 mmol) followed by COMU (1.12 g, 2.62 mmol). The resulting mixture was stirred for 20 min and (R)-2-(aminomethyl)-3-methylbutanoic acid (382.0 mg, 2.91 mmol) was added. The resulting mixture was warmed to room temperature and stirred for an additional 2 h. The reaction mixture was then quenched with H₂O and the aqueous layer was extracted with EtOAc. The combined organic layers were washed with brine, dried over Na₂SO₄, filtered, and concentrated under reduced pressure. The crude residue was purified by reverse phase chromatography (30→70% MeCN/H₂O+0.1% NH₄HCO₃) to afford the desired product (850 mg, 63% yield). LCMS (ESI) m/z: [M−H] calcd for C₂₈H₃₀N₂O₃: 441.22; found 441.2.

### Step 2: Synthesis of methyl (R)-3-methyl-2-(((S)-1-tritylaziridine-2-carboxamido) methyl) butanoate

To a solution of (R)-3-methyl-2-(((S)—N-methyl-1-tritylaziridine-2-carboxamido) methyl) butanoic acid (840.0 mg, 1.90 mmol) in MeOH (5.0 mL) at 0° C. was added TMSCHN₂ (10.0 mL, 0.45 mmol). The resulting mixture was warmed to room temperature and stirred for 2 h, at which point the reaction mixture was concentrated under reduced pressure. The residue was purified by reverse phase chromatography (30→80% MeCN/H₂O+0.1% NH₄HCO₃) to afford the desired product (450 mg, 52% yield). LCMS (ESI) m/z: [M−H] calcd for C₂₉H₃₂N₂O₃: 455.23; found 455.1.

### Step 3: Synthesis of methyl (R)-3-methyl-2-(((S)—N-methyl-1-tritylaziridine-2-carboxamido) methyl) butanoate

To a solution of methyl (R)-3-methyl-2-(((S)-1-tritylaziridine-2-carboxamido) methyl) butanoate (440.0 mg, 0.96 mmol) in THF (5.0 mL) at 0° C. was added NaH (46.25 mg, 1.93 mmol). The resulting mixture was stirred for 30 min and then Mel (1.37 g, 9.65 mmol) was added. The resulting mixture was warmed to room temperature and stirred for an additional 4 h. The reaction mixture was then quenched with H₂O and the aqueous layer was extracted with EtOAc (3×300 mL). The combined organic layers were washed with brine (3×200 mL), dried over Na₂SO₄, filtered, and concentrated under reduced pressure. The residue was purified by reverse phase chromatography (10→90% MeCN/H₂O+0.1% NH₄HCO₃) to afford the desired product (340 mg, 75% yield). LCMS (ESI) m/z: [M+H] calcd for C₃₀H₃₄N₂O₃: 471.26; found 471.3.

### Step 4: Synthesis of (R)-3-methyl-2-(((S)—N-methyl-1-tritylaziridine-2-carboxamido) methyl) butanoic acid

To a solution of methyl (R)-3-methyl-2-(((S)—N-methyl-1-tritylaziridine-2-carboxamido) methyl) butanoate (340.0 mg, 0.72 mmol) in MeOH (3.0 mL) and H₂O (3.0 mL) was added LiOH·H₂O (242.5 mg, 5.78 mmol). The resulting mixture was stirred for 16 h at room temperature and was then acidified to pH 4 with KHSO₄ (1 N). The resulting mixture was extracted with EtOAc (3×300 mL) and the combined organic layers were washed with brine (3×300 mL), dried over Na₂SO₄, filtered, and concentrated under reduced pressure. The residue was purified by reverse phase chromatography (10→80% MeCN/H₂O+0.1% NH₄HCO₃) to afford the desired product (260 mg). LCMS (ESI) m/z: [M+H] calcd for C₂₉H₃₂N₂O₃: 455.23; found 455.1.

### Intermediate A-32. Synthesis of N-methyl-N-(1-((R)-1-tritylaziridine-2-carbonyl) piperidine-4-carbonyl)-L-valine

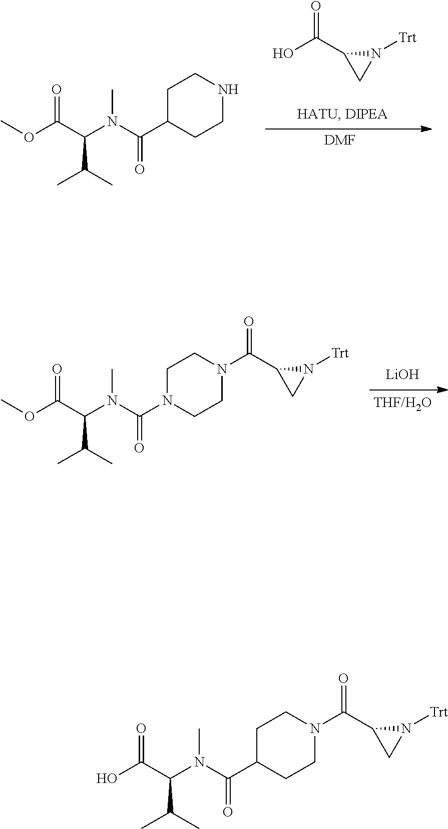

### Step 1: Synthesis of methyl N-methyl-N-(1-((R)-1-tritylaziridine-2-carbonyl) piperidine-4-carbonyl)-L-valinate

To a mixture of methyl N-methyl-N-(piperidine-4-carbonyl)-L-valinate (750 mg, 2.93 mmol) and (R)-1-tritylaziridine-2-carboxylic acid (1.13 g, 3.43 mmol) in DMF (7 mL) at 0° C. was added DIPEA (2.50 mL, 14.62 mmol) followed by HATU (2.20 g, 5.79 mmol) in portions. The resulting mixture was warmed to room temperature and stirred for 3 h. The reaction mixture was diluted with EtOAc (300 mL) and the mixture was washed with brine (2×150 mL), dried with Na₂SO₄, filtered, and concentrated under reduced pressure. Purification by normal phase chromatography (50% EtOAc/hexanes) afforded the desired product (1.5 g, 90.3% yield). LCMS (ESI) m/z: [M+H] calcd for C₃₅H₄₁N₃O₄: 568.32; found 568.3.

### Step 2: Synthesis of N-methyl-N-(1-((R)-1-tritylaziridine-2-carbonyl) piperidine-4-carbonyl)-L-valine

To a solution of methyl N-methyl-N-(1-((R)-1-tritylaziridine-2-carbonyl) piperidine-4-carbonyl)-L-valinate (500 mg, 0.881 mmol) in THF (5 mL) at 0° C. was added a solution of LiOH (111 mg, 2.64 mmol) in H₂O (2.6 mL). The resulting mixture was warmed to room temperature and stirred for 4 h. The reaction mixture was diluted with H₂O (300 mL) and acidified to pH 5 with 1M HCl. The resulting mixture was extracted with DCM (3×100 mL) and the combined organic layers were washed with brine (2×150 mL), dried with Na₂SO₄, filtered, and concentrated under reduced pressure to afford the desired product (600 mg, crude) which was used without further purification. LCMS (ESI) m/z: [M−H] calcd for C₃₄H₃₉N₃O₄: 552.29; found 552.3.

### Intermediate A-33. Synthesis of N-methyl-N-(1-((S)-1-tritylaziridine-2-carbonyl) piperidine-4-carbonyl)-L-valine

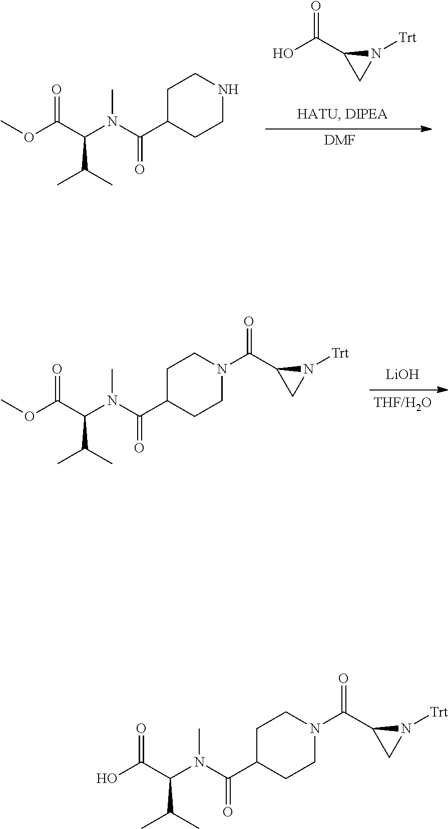

### Step 1: Synthesis of methyl N-methyl-N-(1-((S)-1-tritylaziridine-2-carbonyl) piperidine-4-carbonyl)-L-valinate

To a mixture of methyl N-methyl-N-(piperidine-4-carbonyl)-L-valinate (0.90 g, 3.511 mmol) and(S)-1-tritylaziridine-2-carboxylic acid (2.31 g, 7.022 mmol) in DMF (10 mL) at 0° C. was added DIPEA (3.06 mL, 17.57 mmol) and HATU (2.67 g, 7.022 mmol). The resulting mixture was warmed to room temperature and stirred for 2 h. The reaction mixture was diluted with EtOAc (50 mL) and the mixture was washed with H₂O, brine (100 mL), dried with Na₂SO₄, filtered, and concentrated under reduced pressure. Purification by normal phase chromatography (100% EtOAc) afforded the desired product (1.47 g, 73.7% yield). LCMS (ESI) m/z: [M+H] calcd for C₃₅H₄₁N₃O₄: 568.32; found 568.3.

### Step 2: Synthesis of N-methyl-N-(1-((S)-1-tritylaziridine-2-carbonyl) piperidine-4-carbonyl)-L-valine

To a solution of methyl N-methyl-N-(1-((S)-1-tritylaziridine-2-carbonyl) piperidine-4-carbonyl)-L-valinate (1.0 g, 1.76 mmol) in THF (15 mL) at 0° C. was added a solution of LiOH (370 mg, 8.80 mmol) in H₂O (15 mL). The resulting mixture was warmed to room temperature and stirred for 3 h. The reaction mixture was acidified to pH 6 with 1M HCl. The aqueous layer was extracted with EtOAc (2×50 mL) and the combined organic layers were dried with Na₂SO₄, filtered, and concentrated under reduced pressure to afford the desired product (1.33 g, crude) which was used without further purification. LCMS (ESI) m/z: [M+H] calcd for C₃₄H₃₉N₃O₄: 554.30; found 554.3.

### Intermediate A-34. Synthesis of sodium (R)-1-methyl-5-(1-tritylaziridine-2-carboxamido)-1H-imidazole-2-carboxylate

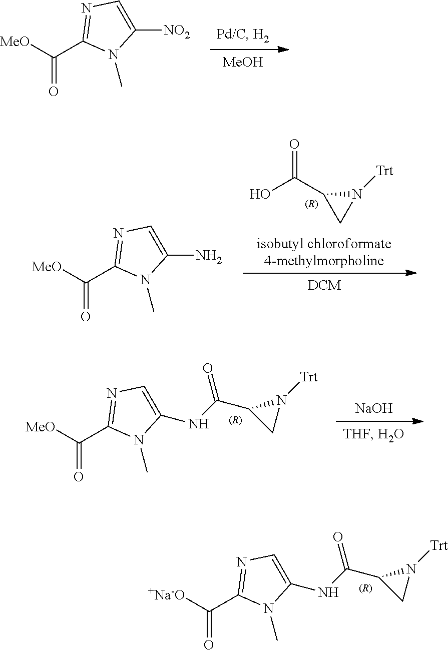

### Step 1: Synthesis of methyl 5-amino-1-methyl-1H-imidazole-2-carboxylate

To a mixture of methyl 1-methyl-5-nitro-1H-imidazole-2-carboxylate (1.0 g, 5.401 mmol) in MeOH (15 mL) was added Pd/C (500 mg). The resulting mixture was placed under an atmosphere of H₂ (1 atm) and stirred for 3 h. The mixture was filtered, the filter cake was washed with MeOH (3×20 mL), and the filtrate was concentrated under reduced pressure to afford the desired product (1.0 g, crude). LCMS (ESI) m/z: [M+H] calcd for C₆H₉N₃O_{2: 156.08}; found 156.1.

### Step 2: Synthesis of methyl (R)-1-methyl-5-(1-tritylaziridine-2-carboxamido)-1H-imidazole-2-carboxylate

To a mixture of (R)-1-tritylaziridine-2-carboxylic acid (2.55 g, 7.741 mmol) in DCM (12.0 mL) at 0° C. was added a solution of isobutyl chloroformate (845.06 mg, 6.187 mmol) and N-methylmorpholine (1.04 g, 10.282 mmol) in DCM in portions over 30 min. To the resulting mixture was added methyl 5-amino-1-methyl-1H-imidazole-2-carboxylate (800.0 mg, 5.156 mmol). The mixture was stirred at room temperature overnight then diluted with DCM (300 mL) and washed with H₂O (3×100 mL). The organic layer was washed with brine (2×150 mL), dried with Na₂SO₄, filtered, and concentrated under reduced pressure. Purification by silica gel column chromatography (25% EtOAc/hexanes) afforded the desired product (1.2 g, 49.9% yield). LCMS (ESI) m/z: [M+H] calcd for C₂₈H₂₆N₄O₃: 467.21; found 467.2.

### Step 3: Synthesis of sodium (R)-1-methyl-5-(1-tritylaziridine-2-carboxamido)-1H-imidazole-2-carboxylate

To a mixture of methyl (R)-1-methyl-5-(1-tritylaziridine-2-carboxamido)-1H-imidazole-2-carboxylate (300 mg, 0.643 mmol) in THF (3 mL) was added a solution of NaOH (38.58 mg, 0.965 mmol) in H₂O. The resulting mixture was stirred for 2 h and then concentrated under reduced pressure to afford the desired product (400 mg, crude). LCMS (ESI) m/z: [M+H] calcd for C₂₇H₂₄N₄O₃: 453.19; found 453.2.

### Intermediate A-35. Synthesis of(S)-1-methyl-5-(1-tritylaziridine-2-carboxamido)-1H-imidazole-2-carboxylic acid

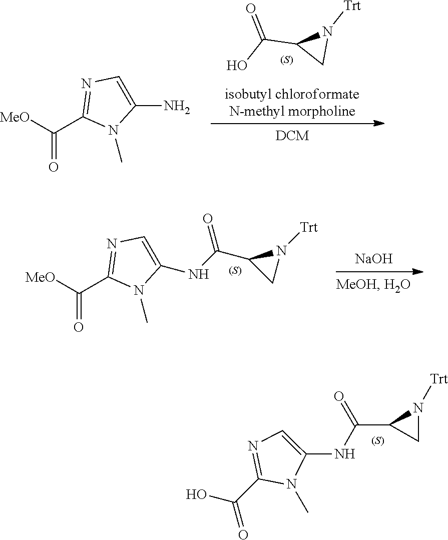

### Step 1: Synthesis of methyl(S)-1-methyl-5-(1-tritylaziridine-2-carboxamido)-1H-imidazole-2-carboxylate

To a mixture of(S)-1-tritylaziridine-2-carboxylic acid (1.18 g, 3.577 mmol) in DCM (15 mL) at 0° C. was added isobutyl chloroformate (423.41 mg, 3.100 mmol) and N-methylmorpholine (0.39 mL, 3.862 mmol) dropwise. The resulting mixture was stirred at 0° C. for 1 h then methyl 5-amino-1-methyl-1H-imidazole-2-carboxylate (370.0 mg, 2.385 mmol) was added. The mixture was warmed to room temperature and stirred overnight. The reaction was quenched with sat. NaHCO₃ at 0° C. and the resulting mixture was extracted with DCM (2×100 mL). The combined organic layers were washed with brine (150 mL), dried with Na₂SO₄, filtered, and concentrated under reduced pressure. Purification by silica gel column chromatography (100% EtOAc) afforded the desired product (380 mg, 34.2% yield). LCMS (ESI) m/z: [M+H] calcd for C₂₈H₂₆N₄O₃: 467.21; found 467.3.

### Step 2: Synthesis of(S)-1-methyl-5-(1-tritylaziridine-2-carboxamido)-1H-imidazole-2-carboxylic acid

To a mixture of methyl(S)-1-methyl-5-(1-tritylaziridine-2-carboxamido)-1H-imidazole-2-carboxylate (380.0 mg, 0.815 mmol) in MeOH (5 mL) at 0° C. was added NaOH (146.60 mg, 3.665 mmol) in H₂O (3.6 mL) dropwise. The resulting mixture was warmed to room temperature and stirred for 6 h then was acidified to pH 6 with 1M HCl. The resulting mixture was extracted with DCM (2×100 mL), and the combined organic layers were washed with brine (150 mL), dried with Na₂SO₄, filtered, and concentrated under reduced pressure to afford the desired product (350 mg, crude). LCMS (ESI) m/z: [M−H] calcd for C₂₇H₂₄N₄O₃: 451.17; found 451.1.

### Intermediate A-36 and A-37. Synthesis of (R)—N-methyl-N-((1-methylaziridin-2-yl) sulfonyl) glycine and(S)—N-methyl-N-((1-methylaziridin-2-yl) sulfonyl) glycine

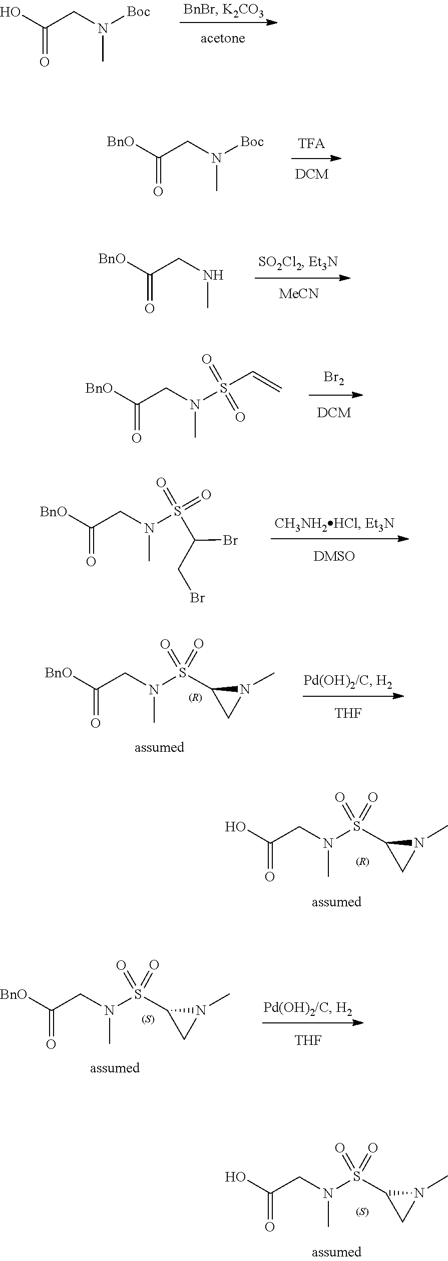

### Step 1: Synthesis of benzyl N-(tert-butoxycarbonyl)-N-methylglycinate

To a stirred mixture of [(tert-butoxycarbonyl) (methyl) amino]acetic acid (15. g, 79.28 mmol) in acetone (150 mL) was added BnBr (14.14 mL, 82.70 mmol) and K₂CO₃ (21.91 g, 158.55 mmol) in portions at 0° C. . . . The resulting mixture was stirred for 4 h at room temperature. The resulting mixture was filtered, the filter cake was washed with acetone (3×100 mL), and the filtrate was concentrated under reduced pressure. The residue was purified by silica gel column chromatography (33% EtOAc/pet. ether) to afford the desired product (15.2 g, 68.6% yield). LCMS (ESI) m/z: [M+Na] calcd for C₁₅H₂₁NO₄: 302.14; found 302.0.

### Step 2: Synthesis of benzyl methylglycinate

To a stirred solution of benzyl N-(tert-butoxycarbonyl)-N-methylglycinate (10.0 g, 35.80 mmol) in DCM (100 mL) was added TFA (50 mL) dropwise at 0° C. The resulting mixture was stirred for 1 h at 0° C. and then the resulting mixture was concentrated under reduced pressure to afford the desired product (7.80 g, crude). LCMS (ESI) m/z: [M+H] calcd for C₁₀H₁₃NO_{2: 180.10}; found 179.1.

### Step 3: Synthesis of benzyl N-methyl-N-(vinylsulfonyl) glycinate

To a solution of benzyl methylglycinate (15.60 g, 87.04 mmol) and Et₃N (36.4 mL, 261.1 mmol) in MeCN (300 mL) at −70° C. was added a solution of 2-chloroethanesulfonyl chloride (17.03 g, 104.47 mmol) in MeCN (150 mL). The resulting mixture was warmed to room temperature and stirred for 20 min. The reaction mixture was cooled −50° C. and additional Et₃N (36.4 mL, 261.1 mmol) was added to reaction mixture. The reaction mixture was warmed to room temperature and stirred for 1 h. The reaction was then quenched with H₂O at 0° C. The mixture was acidified to pH 6 with 1 M HCl aq and was then extracted with DCM (800 mL), dried over Na₂SO₄, filtered, and concentrated under reduced pressure. The residue was purified by silica gel column chromatography (33% EtOAc/pet. ether) to afford the desired product (7.53 g, 32.1% yield). LCMS (ESI) m/z: [M+H₂O] calcd for C₁₂H₁₅NO₄S: 287.08; found 287.2.

### Step 4: Synthesis of benzyl N-((1,2-dibromoethyl) sulfonyl)-N-methylglycinate

To a solution of benzyl N-methyl-N-(vinylsulfonyl) glycinate (5.58 g, 20.7 mmol) in DCM (50 mL) at −20° C. was added a solution of Br₂ (1.06 mL, 6.64 mmol) in DCM (10 mL). The resulting mixture was warmed to room temperature and stirred overnight. The reaction mixture was then cooled to 0° C. and quenched with sat. aq. Na₂S₂O₃ (30 mL). The resulting mixture was washed with sat. aq. Na₂HCO₃ and then extracted with DCM (2×200 mL), the combined organic layers dried over Na₂SO₄, filtered, and concentrated under reduced pressure. The residue was purified by silica gel column chromatography (33% EtOAc/pet. ether) to afford the desired product (5.1 g, 57.4% yield). LCMS (ESI) m/z: [M+H₂O] calcd for C₁₂H₁₅Br₂NO₄S: 444.92; found 444.9.

### Step 5: Synthesis of benzyl (R)—N-methyl-N-((1-methylaziridin-2-yl) sulfonyl) glycinate and benzyl(S)—N-methyl-N-((1-methylaziridin-2-yl) sulfonyl) glycinate

To a stirred solution of benzyl N-((1,2-dibromoethyl) sulfonyl)-N-methylglycinate (7.20 g, 16.78 mmol) and methylamine hydrochloride (3.39 g, 50.2 mmol) in DMSO (750 mL) was added Et₃N (23.32 mL, 230.47 mmol). The resulting mixture was stirred for 2 h at room temperature then heated to 75° C. and stirred overnight. The reaction mixture was cooled to room temperature and extracted with EtOAc (2×1000 mL). The combined organic layers were washed with H₂O (1500 mL) and brine (1500 mL), dried over Na₂SO₄, filtered, and concentrated under reduced pressure. The residue was purified by silica gel column chromatography (EtOAc) to afford a mixture of diastereomers. The diastereomers were separated by prep-SFC(10% EtOH/Hex) to afford benzyl (R)—N-methyl-N-((1-methylaziridin-2-yl) sulfonyl) glycinate (500 mg, 31.3% yield) and benzyl(S)—N-methyl-N-((1-methylaziridin-2-yl) sulfonyl) glycinate (600 mg, 37.5% yield). LCMS (ESI) m/z: [M+H] calcd for C₁₃H₁₈N₂O₄S: 299.11; found 299.0.

### Step 6: Synthesis of (R)—N-methyl-N-((1-methylaziridin-2-yl) sulfonyl) glycine

A suspension of benzyl (R)—N-methyl-N-((1-methylaziridin-2-yl) sulfonyl) glycinate (300.0 mg) and Pd (OH) 2/C(150.0 mg) in THF at room temperature was stirred under an atmosphere of hydrogen (1 atm) for 3 h. The mixture was filtered, the filter cake was washed with MeOH (3×20 mL), and the filtrate was concentrated under reduced pressure to afford the desired product (206 mg, crude). LCMS (ESI) m/z: [M+H] calcd for C₆H₁₂N₂O₄S: 209.06; found 209.0.

### Step 7: Synthesis of(S)—N-methyl-N-((1-methylaziridin-2-yl) sulfonyl) glycine

A suspension of benzyl (R)—N-methyl-N-((1-methylaziridin-2-yl) sulfonyl) glycinate (300.0 mg, 1.01 mmol) and Pd (OH) 2/C(150.0 mg) in THF at room temperature was stirred under an atmosphere of hydrogen (1 atm) for 3 h. The mixture was filtered, the filter cake was washed with MeOH (3×20 mL), and the filtrate was concentrated under reduced pressure to afford the desired product (216 mg, crude). LCMS (ESI) m/z: [M+H] calcd for C₆H₁₂N₂O₄S: 209.06; found 209.1.

### Intermediate A-38. Synthesis of (2S,3S)-1-(tert-butylsulfinyl)-3-cyclopropylaziridine-2-carboxylic acid

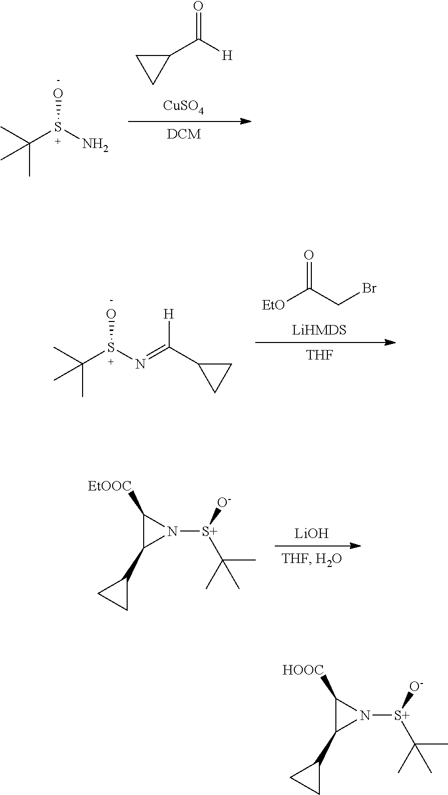

### Step 1: Synthesis of (E)-N-(cyclopropylmethylene)-2-methylpropane-2-sulfinamide

To a suspension of(S)-2-methylpropane-2-sulfinamide (4.0 g, 33.0 mmol) and CuSO₄ (15.80 g, 99.01 mmol) in DCM (200.0 mL) was added cyclopropanecarbaldehyde (4.63 g, 66.0 mmol). The resulting mixture was stirred overnight and was then filtered, the filter cake was washed with DCM (3×100 mL), and the filtrate was concentrated under reduced pressure to afford the desired product (3.5 g, 61.2% yield). LCMS (ESI) m/z: [M+H] calcd for C₈H₁₅NOS: 174.10; found 174.1.

### Step 2: Synthesis of ethyl (2S,3S)-1-(tert-butylsulfinyl)-3-cyclopropylaziridine-2-carboxylate

To a solution of ethyl bromoacetate (481.91 mg, 2.886 mmol) in THF (5.0 mL) at −78° C. was added LiHMDS (2.90 mL, 2.90 mmol). The resulting mixture was stirred for 2 h at −78° C. and then a solution of (E)-N-(cyclopropylmethylene)-2-methylpropane-2-sulfinamide (250.0 mg, 1.443 mmol) was added. The resulting mixture was stirred for 2 h at −78° C. and was then was then quenched with H₂O at 0° C. The aqueous layer was extracted with EtOAc (3×50 mL), and the combined organic layers were dried over Na₂SO₄, filtered, and concentrated under reduced pressure. The residue was purified by prep-TLC(17% EtOAc/pet. ether) to afford the desired product (250 mg, 66.8% yield). LCMS (ESI) m/z: [M+H] calcd for C₁₂H₂₁NO₃S: 260.13; found 260.1.

### Step 3: Synthesis of (2S,3S)-1-(tert-butylsulfinyl)-3-cyclopropylaziridine-2-carboxylic acid

A solution of ethyl (2S,3S)-1-(tert-butylsulfinyl)-3-cyclopropylaziridine-2-carboxylate (500.0 mg, 1.928 mmol) in THF (2.0 mL) and H₂O (2.0 mL) at 0° C. was added LiOH·H₂O (121.34 mg, 2.89 mmol). The reaction mixture was stirred for 1 h and was then acidified to pH 6 with 1 M HCl (aq.). The resulting mixture was extracted with EtOAc (2×10 mL) and the combined organic layers were washed with brine (10 mL), dried over Na₂SO₄, filtered, and the filtrate was concentrated under reduced pressure to afford the desired product (400 mg, 89.7% yield). LCMS (ESI) m/z: [M+H] calcd for C₁₀H₁₇NO₃S: 232.10; found 232.0.

### Intermediate A-39. Synthesis of (2R,3R)-3-(methoxymethyl)-1-tritylaziridine-2-carboxylic acid

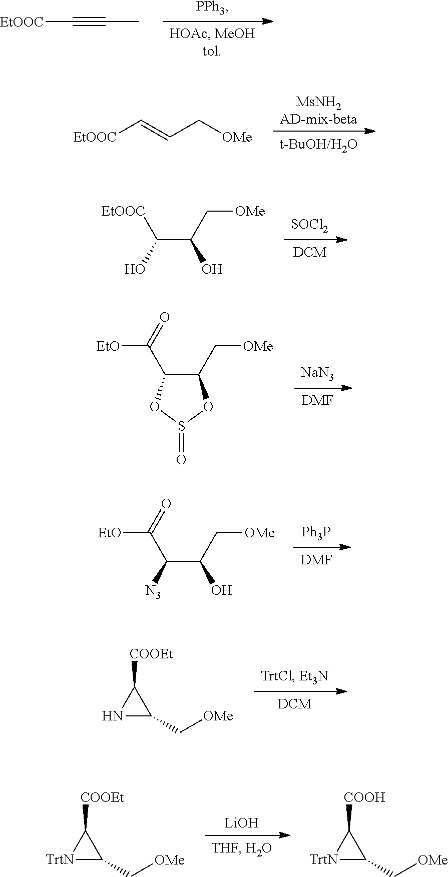

### Step 1: Synthesis of ethyl (E)-4-methoxybut-2-enoate

To a solution of ethyl but-2-ynoate (10.0 g, 89.18 mmol) in MeOH (8.80 mL, 118.594 mmol) and HOAc (1.05 mL, 18.3 mmol) was added a solution of PPh₃ (1.20 g, 4.58 mmol) in toluene (60.0 mL). The resulting solution heated to 110° C. and stirred overnight. The reaction mixture was cooled to room temperature and was then diluted with H₂O (60 mL). The resulting solution was extracted with EtOAc (2×60), and the combined organic layers were washed with brine (2×20 mL), dried over Na₂SO₄, filtered, and concentrated under reduced pressure. The residue was purified by silica gel column chromatography (9% EtOAc/pet. ether) to afford the desired product (4.9 g, 38.1% yield). LCMS (ESI) m/z: [M+H] calcd for C₇H₁₂O₃: 145.09; found 144.9.

### Step 2: Synthesis of ethyl (2S,3R)-2,3-dihydroxy-4-methoxybutanoate

To a solution of ethyl (E)-4-methoxybut-2-enoate (5.0 g, 34.68 mmol), and methanesulfonamide (3.30 g, 34.68 mmol) in t-BuOH (150.0 mL) and H₂O (100.0 mL) was added AD-mix-β (48.63 g, 62.43 mmol). The resulting solution was heated to 30° C. and stirred overnight. The solution was then cooled to room temperature and adjusted to pH 2 with KHSO₄. The resulting solution was extracted with EtOAc (2×100 mL) and the combined organic layers were dried over Na₂SO₄, filtered, and concentrated under reduced pressure to afford the desired product (1.28 g, crude). LCMS (ESI) m/z: [M+H] calcd for C₇H₁₄O₅: 179.09; found 179.0.

### Step 3: Synthesis of ethyl (4S,5R)-5-(methoxymethyl)-1,3,2-dioxathiolane-4-carboxylate 2-oxide

To a solution of ethyl (2S,3R)-2,3-dihydroxy-4-methoxybutanoate (4.10 g, 23.01 mmol) in DCM (20.0 mL) at 0° C. was added SOCl₂ (5.47 g, 45.9 mmol). The resulting mixture was heated to 50° C. and stirred for 3 h. The reaction mixture was then cooled to room temperature and concentrated under reduced pressure to afford the desired product (4.0 g, crude).

### Step 4: Synthesis of ethyl (2R,3S)-2-azido-3-hydroxy-4-methoxybutanoate

To a solution of ethyl (4S,5R)-5-(methoxymethyl)-1,3,2-dioxathiolane-4-carboxylate 2-oxide (4.0 g crude, 17.84 mmol) in DMF (20.0 mL) at 0° C. was added NaN₃ (5.80 g, 89.22 mmol). The resulting mixture was heated to 35° C. and stirred overnight. The reaction mixture was then diluted with H₂O (200 mL) and was extracted with EtOAc (3×50 mL). The combined organic layers were washed with brine (3×50 mL), dried over Na₂SO₄, filtered, and concentrated under reduced pressure. The residue was purified by silica gel column chromatography (17% EtOAc/pet. ether) to afford the desired product (1.0 g, 27.6% yield). LCMS (ESI) m/z: [M+H] calcd for C₇H₁₃N₃O₄: 204.10; found 204.0.

### Step 5: Synthesis of ethyl (2R,3R)-3-(methoxymethyl) aziridine-2-carboxylate

To a solution of ethyl (2R,3S)-2-azido-3-hydroxy-4-methoxybutanoate (1.0 g, 4.92 mmol) in DMF (10 mL) at 0° C. was added PPh₃ (1.29 g, 4.92 mmol) in portions over 30 min. The reaction solution was then warmed to room temperature and stirred for 30 min. The reaction mixture was then heated to 85° C. and stirred until the reaction was complete. The reaction mixture was then concentrated under reduced pressure and purified by prep-TLC(33% EtOAc/pet. ether) to afford the desired product (480 mg, 61.3% yield). LCMS (ESI) m/z: [M+H] calcd for C₇H₁₃NO₃: 160.10; found 160.1.

### Step 6: Synthesis of ethyl (2R,3R)-3-(methoxymethyl)-1-tritylaziridine-2-carboxylate

To a solution of ethyl (2R,3R)-3-(methoxymethyl) aziridine-2-carboxylate (480.0 mg, 3.02 mmol) and Et₃N (2.1 mL, 15.0 mmol) in DCM (10 mL) at 0° C. was added Trt-Cl (1.681 g, 6.031 mmol). The resulting mixture was warmed to room temperature and stirred for 2 h. The mixture was concentrated then concentrated under reduced pressure and the residue was purified by prep-TLC (5% EtOAc/pet. ether) to afford the desired product (700 mg, crude).

### Step 7: Synthesis of (2R,3R)-3-(methoxymethyl)-1-tritylaziridine-2-carboxylic acid

To a solution of ethyl (2R, 3R)-3-(methoxymethyl)-1-(triphenylmethyl) aziridine-2-carboxylate (200.0 mg, 0.498 mmol) in THF (5.0 mL) and H₂O (5 mL) was added LiOH·H₂O (41.81 mg, 0.996 mmol). The resulting solution was stirred at room temperature for 24 h. The mixture was then diluted with H₂O (10 mL) and extracted with EtOAc (20 mL). The aqueous layer was then acidified to pH 7 with sat. aq. NH₄Cl and extracted with EtOAc (2×10 mL). The combined organic layers were dried over Na₂SO₄, filtered, and concentrated under reduced pressure to afford the desired product (60 mg, 32.3% yield). LCMS (ESI) m/z: [M−H] calcd for C₂₄H₂₃NO_{3: 372.16}; found 372.1.

### Intermediate A-40. Synthesis of (25,3S)-1-(tert-butylsulfinyl)-3-(4-methoxyphenyl) aziridine-2-carboxylic acid

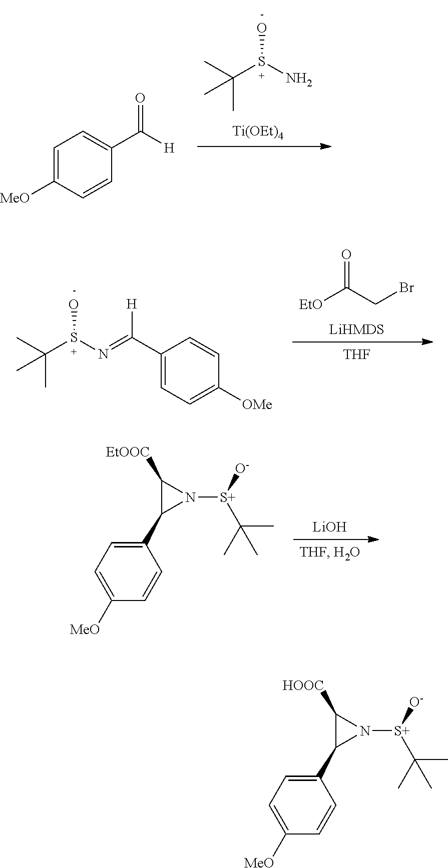

### Step 1: (E)-N-(4-methoxybenzylidene)-2-methylpropane-2-sulfinamide

A solution of(S)-2-methylpropane-2-sulfinamide (2.50 g) and anisaldehyde (2.81 g) in Ti(OEt)₄ (20.0 mL) was stirred at 70° C. for 1 h. The resulting mixture was cooled to room temperature, diluted with EtOAc (60 mL), and then poured into H₂O. The mixture was filtered, and the filter cake was washed with EtOAc (3×50 mL). The resulting mixture was extracted with EtOAc (3×50 mL) and the combined organic layers were washed with brine (50 mL), dried over Na₂SO₄, filtered, and concentrated under reduced pressure. The residue was purified by silica gel column chromatography (25% EtOAc/pet. ether) to afford the desired product (4 g, 81.0% yield). LCMS (ESI) m/z: [M+H] calcd for C₁₂H₁₇NO₂S: 240.11; found 240.1.

### Step 2: Synthesis of ethyl (2S,3S)-1-(tert-butylsulfinyl)-3-(4-methoxyphenyl) aziridine-2-carboxylate

To a solution of ethyl 2-bromoacetate (5.60 g, 33.5 mmol) in THF (100 mL) at −78° C. was added LiHMDS (1M in THF, 34 mL, 33.473 mmol). After 30 min a solution of (E)-N-(4-methoxybenzylidene)-2-methylpropane-2-sulfinamide (4 g, 16.74 mmol) in THF (20 mL) was added. The resulting mixture was stirred at −78° C. for additional 3 h. The reaction was then quenched with sat. aq. NH₄Cl. The mixture was extracted with EtOAc (3×100 mL) and the combined organic layers were washed with brine (50 mL), dried over Na₂SO₄, filtered, and concentrated under reduced pressure. The residue was purified by silica gel chromatography (25% EtOAc/pet. ether) to afford the desired product (2.7 g, 49.6% yield). LCMS (ESI) m/z: [M+H] calcd for C₁₆H₂₃NO₄S: 326.14; found 326.1.

### Step 3: Synthesis of (2S,3S)-1-(tert-butylsulfinyl)-3-(4-methoxyphenyl) aziridine-2-carboxylic acid

To a solution of ethyl (2S,3S)-1-(tert-butylsulfinyl)-3-(4-methoxyphenyl) aziridine-2-carboxyla the (800.0 mg, 2.68 mmol) in THF (2.0 mL) at 0° C. was added a solution of LiOH·H₂O (309.46 mg, 7. 38 mmol) in H₂O (3.0 mL). The resulting mixture was warmed to room temperature and stirred for 4 h. The mixture was then acidified to pH 6 with sat. aq. NH₄Cl and then extracted with EtOAc (3×50 mL). The combined organic layers were dried over Na₂SO₄, filtered. and concentrated under reduce d pressure to afford the desired product (690 mg, 94.4% yield). LCMS (ESI) m/z: [M−H] calcd for C ₁₄H₁₉NO₄S: 296.10; found 296.2.

### Intermediate A-41. Synthesis of (2S,3R)-3-(4-methoxyphenyl) aziridine-2-carboxylic acid

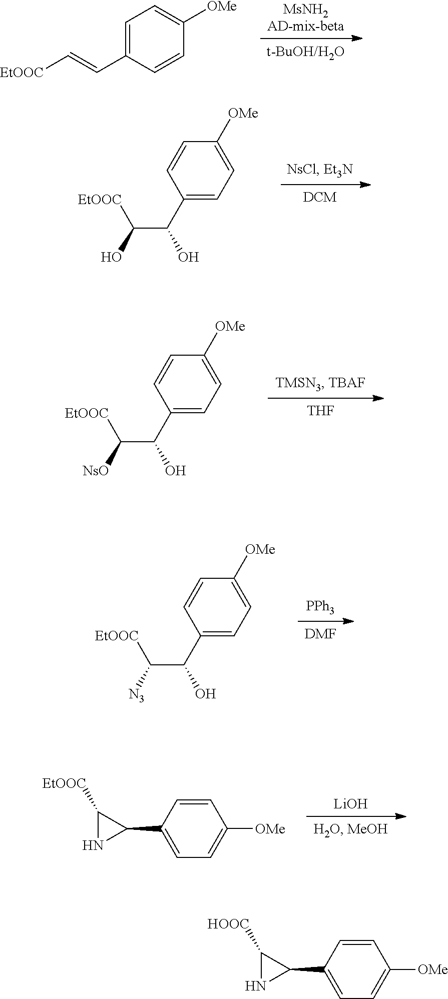

### Step 1: Synthesis of ethyl (2R,3S)-2,3-dihydroxy-3-(4-methoxyphenyl) propanoate

To a solution of ethyl p-methoxycinnamate (5.0 g, 24.24 mmol) in (BuOH (70.0 mL) and H₂O (70.0 mL) at 0° C. was added AD-mix-α (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL) and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88.1% yield).

### Step 2: Synthesis of ethyl (2R,3S)-3-hydroxy-3-(4-methoxyphenyl)-2-(((4-nitrophenyl) sulfonyl)oxy) propanoate

To a solution of ethyl (2R,3S)-2,3-dihydroxy-3-(4-methoxyphenyl) propanoate (3.0 g, 12.49 mmol) and Et₃N (0.174 mL, 1.249 mmol) in DCM (30.0 mL) at 0° C. was added 4-nitrobenzenesulfonyl chloride (2.76 g, 12.49 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC(50% EtOAc/pet. ether) to afford the desired product (3.8 g, 68.0% yield). LCMS (ESI) m/z: [M+Na] calcd for C₁₈H₁₉NO₉S: 448.07; found 448.2.

### Step 3: Synthesis of ethyl (2S,3S)-2-azido-3-hydroxy-3-(4-methoxyphenyl) propanoate

To a solution of ethyl (2R,3S)-3-hydroxy-3-(4-methoxyphenyl)-2-(((4-nitrophenyl) sulfonyl)oxy) propanoate (1.20 g, 2.82 mmol) in THF at 0° C. was added TBAF (1M in THF, 5.64 mL, 5.64 mmol) and TMSN₃ (648.79 mg, 5.64 mmol). The resulting mixture was heated to at 60° C. and stirred for 16 h. The reaction was then cooled to at 0° C. and quenched with sat. aq. NH₄Cl. The mixture was extracted with EtOAc (3×100 mL) and the combined organic layers were washed with H₂O (2×100 mL), dried over Na₂SO₄, filtered, and concentrated under reduced pressure. The residue was purified by prep-TLC(33% EtOAc/pet. ether) to afford the desired product (540 mg, 70.7% yield).

### Step 4: Synthesis of ethyl (2S,3R)-3-(4-methoxyphenyl) aziridine-2-carboxylate

To a solution of ethyl (2S,3S)-2-azido-3-hydroxy-3-(4-methoxyphenyl) propanoate (440.0 mg, 1.659 mmol) in DMF was added PPh₃ (522.06 mg, 1.99 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC(25% EtOAc/pet. ether) to afford the desired product (200 mg, 51.8% yield). LCMS (ESI) m/z: [M+H] calcd for C₁₂H₁₅NO₃: 222.12; found 222.1.

### Step 5: Synthesis of (2S,3R)-3-(4-methoxyphenyl) aziridine-2-carboxylic acid

To a solution of ethyl (2S,3R)-3-(4-methoxyphenyl) aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL) and the combined organic layers were washed with H₂O (2×100 mL), dried over Na₂SO₄, filtered, and concentrated under reduced pressure to afford the desired product (180 mg, 97.9% yield). LCMS (ESI) m/z: [M−H] calcd for C₁₀H₁₁NO₃: 192.07; found 192.0.

### Intermediate A-42. Synthesis of (2R,3S)-3-(4-methoxyphenyl) aziridine-2-carboxylic acid

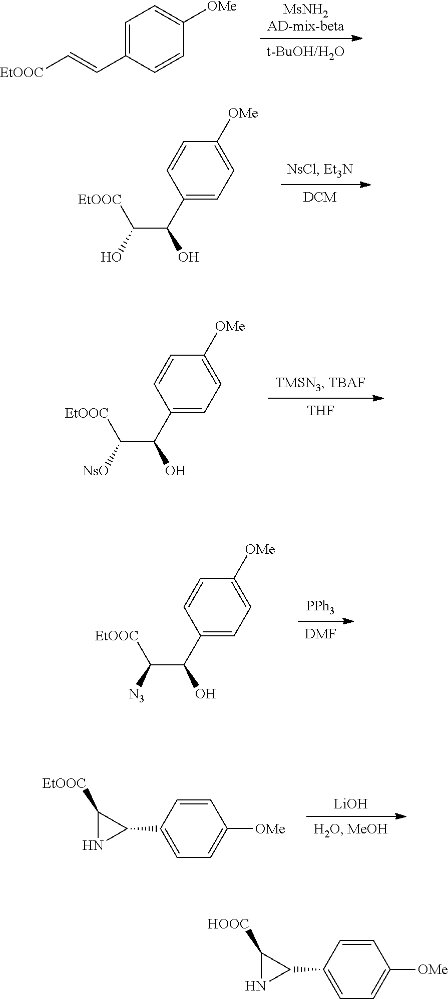

### Step 1: Synthesis of ethyl (2S,3R)-2,3-dihydroxy-3-(4-methoxyphenyl) propanoate

To a solution of ethyl p-methoxycinnamate (5.0 g, 24.24 mmol) in (BuOH (70.0 mL) and H₂O (70.0 mL) at 0° C. was added AD-mix-β (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL) and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88.1% yield).

### Step 2: Synthesis of ethyl (2S,3R)-3-hydroxy-3-(4-methoxyphenyl)-2-(((4-nitrophenyl) sulfonyl)oxy) propanoate

To a solution of ethyl (2S,3R)-2,3-dihydroxy-3-(4-methoxyphenyl) propanoate (5.80 g, 24.14 mmol) and Et₃N (10.1 mL, 72.42 mmol) in DCM (30.0 mL) at 0° C. was added 4-nitrobenzenesulfonyl chloride (5.34 g, 24.1 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC(50% EtOAc/pet. ether) to afford the desired product (7.2 g, 67.0% yield). LCMS (ESI) m/z: [M+H] calcd for C₁₈H₁₉NO₉S: 426.09; found 426.2.

### Step 3: Synthesis of ethyl (2R,3R)-2-azido-3-hydroxy-3-(4-methoxyphenyl) propanoate

To a solution of ethyl (2S,3R)-3-hydroxy-3-(4-methoxyphenyl)-2-(((4-nitrophenyl) sulfonyl)oxy) propanoate (5.0 g, 11.75 mmol) in THF at 0° C. was added TBAF (1M in THF, 23.5 mL, 23.51 mmol) and TMSN₃ (2.7 g, 23.5 mmol). The resulting mixture was heated to at 60° C. and stirred for 16 h. The reaction was then cooled to at 0° C. and quenched with sat. aq. NH₄Cl. The mixture was extracted with EtOAc (3×100 mL) and the combined organic layers were washed with H₂O (2×100 mL), dried over Na₂SO₄, filtered, and concentrated under reduced pressure. The residue was purified by prep-TLC(33% EtOAc/pet. ether) to afford the desired product (2.3 g, 70.1% yield).

### Step 4: Synthesis of ethyl (2R,3S)-3-(4-methoxyphenyl) aziridine-2-carboxylate

To a solution of ethyl (2R,3R)-2-azido-3-hydroxy-3-(4-methoxyphenyl) propanoate (2.30 g, 8.67 mmol) in DMF was added PPh₃ (2.73 g, 10.4 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC(25% EtOAc/pet. ether) to afford the desired product (1.6 g, 79.2% yield). LCMS (ESI) m/z: [M+H] calcd for C₁₂H₁₅NO₃: 222.12; found 222.1.

### Step 5: Synthesis of (2R,3S)-3-(4-methoxyphenyl) aziridine-2-carboxylic acid

To a solution of ethyl (2S,3R)-3-(4-methoxyphenyl) aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL) and the combined organic layers were washed with H₂O (2×100 mL), dried over Na₂SO₄, filtered, and concentrated under reduced pressure to afford the desired product (180 mg, 97.9% yield). LCMS (ESI) m/z: [M−H] calcd for C₁₀H₁₁NO₃: 192.07; found 192.0.

### Intermediate A-43. Synthesis of (2S,3S)-1-((S)-tert-butylsulfinyl)-3-phenylaziridine-2-carboxylic acid

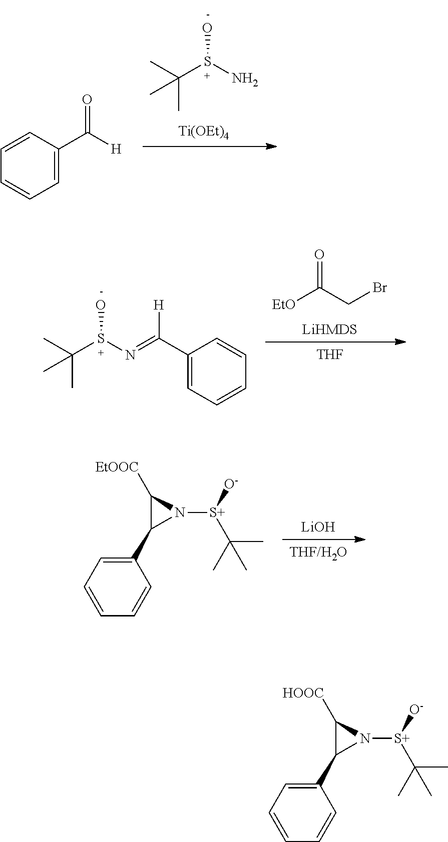

### Step 1: Synthesis of (S,E)-N-benzylidene-2-methylpropane-2-sulfinamide

A solution of(S)-2-methylpropane-2-sulfinamide (2.50 g, 20.6 mmol), titanium ethoxide (9.41 g, 41.25 mmol) and benzaldehyde (2.19 g, 20.7 mmol) was heated at 70° C. for 1 h, cooled, and diluted with H₂O (250 mL). The aqueous layer was extracted with EtOAc (3×80 mL) and the combined organic layers were washed with brine (2×100 mL), dried with Na₂SO₄, filtered and concentrated under reduced pressure to afford the desired product (4.3 g, crude) which was used without further purification. LCMS (ESI) m/z: [M+H] calcd for C₁₁H₁₅NOS: 210.10; found 210.2.

### Step 2: Synthesis of ethyl (2S,3S)-1-((S)-tert-butylsulfinyl)-3-phenylaziridine-2-carboxylate

To a solution of ethyl bromoacetate (798 mg, 4.78 mmol) in THF (15 mL) at −78° C. was added LiHMDS (1M in THF, 4.78 mL, 4.78 mmol). After 1 h, (S,E)-N-benzylidene-2-methylpropane-2-sulfinamide (500 mg, 2.39 mmol) in THF (5 mL) was added in portions over 20 min. The reaction mixture was stirred at −78° C. for 2 h and then quenched by the addition of sat. NH₄Cl. The aqueous layer was extracted with EtOAc (3×40 mL) and the combined organic layers were washed with brine (2×30 mL), dried with Na₂SO₄, filtered, and concentrated under reduced pressure. Purification by reverse phase chromatography (30→60% MeCN/H₂O, 0.1% HCO₂H) afforded the desired product (480 mg, 61% yield). LCMS (ESI) m/z: [M+H] calcd for C₁₅H₂₁NO₃S: 296.13; found 296.2.

### Step 3: Synthesis (2S,3S)-1-((S)-tert-butylsulfinyl)-3-phenylaziridine-2-carboxylic acid

To a solution of ethyl (2S,3S)-1-((S)-tert-butylsulfinyl)-3-phenylaziridine-2-carboxylate (600 mg, 2.03 mmol) in THF (4.0 mL) at 0° C. was added a solution of LiOH (97.2 mg, 4.06 mmol) in H₂O (4.0 mL). The resulting mixture was stirred for 2 h at 0° C. and then acidified to pH 5 with 1 M HCl. The aqueous layer was extracted with EtOAc (3×40 mL) and the combined organic layers were washed with brine (2×20 mL), dried with Na₂SO₄, filtered, and concentrated under reduced pressure to afford the desired compound (450 mg, crude) which was used without further purification. LCMS (ESI) m/z: [M+H] calcd for C₁₃H₁₇NO₃S: 268.10; found 268.1.

### Intermediate A-44. Synthesis of (2R,3R)-1-((R)-tert-butylsulfinyl)-3-phenylaziridine-2-carboxylic acid

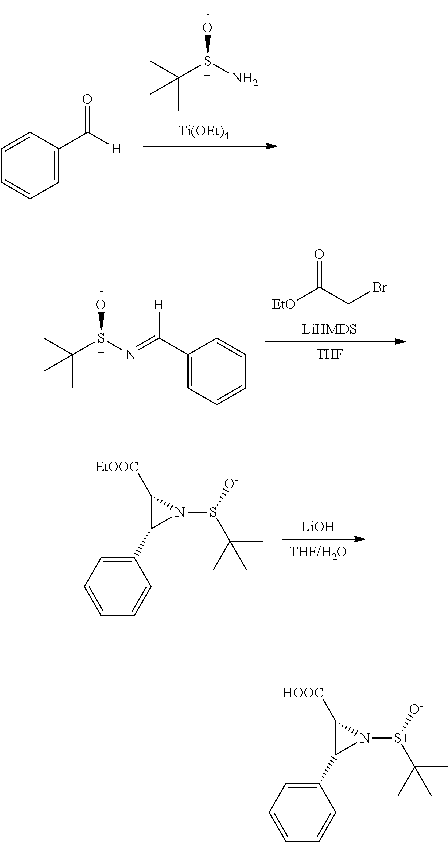

### Step 1: Synthesis (R,E)-N-benzylidene-2-methylpropane-2-sulfinamide

A solution (R)-2-methylpropane-2-sulfinamide (2.50 g, 20.6 mmol), titanium tetraethoxide (9.41 g, 41.3 mmol) and benzaldehyde (2.19 g, 20.6 mmol) was heated 70° C. for 1 h, cooled, and diluted with H₂O (250 mL). The aqueous layer was extracted with EtOAc (3×90 mL) and the combined organic layers were washed with brine (2×100 mL), dried with Na₂SO₄, filtered and concentrated under reduced pressure to afford the desired product (4.2 g, crude) which was used without further purification. LCMS (ESI) m/z: [M+H] calcd for C₁₁H₁₅NOS: 210.10; found 210.1.

### Step 2: Synthesis of ethyl (2R,3R)-1-((R)-tert-butylsulfinyl)-3-phenylaziridine-2-carboxylate

To a solution of ethyl bromoacetate (6.38 g, 38.2 mmol) in THF (150 mL) at −78° C. was added LiHMDS (1M in THF, 7.19 mL, 42.9 mmol). After 1 h, (R,E)-N-benzylidene-2-methylpropane-2-sulfinamide (4.0 g, 19.1 mmol) in THF (50 mL) was added in portions over 20 min. The reaction mixture was stirred at −78° C. for 2 h and then quenched by the addition of sat. NH₄Cl. The aqueous layer was extracted with EtOAc (3×80 mL) and the combined organic layers were washed with brine (2×60 mL), dried with Na₂SO₄, filtered and concentrated under reduced pressure. Purification by reverse phase chromatography (30→60% MeCN/H₂O, 0.1% HCO₂H) afforded the desired product (3.9 g, 62% yield). LCMS (ESI) m/z: [M+H] calcd for C₁₅H₂₁NO₃S: 296.13; found 296.2.

### Step 3: Synthesis (2R,3R)-1-((R)-tert-butylsulfinyl)-3-phenylaziridine-2-carboxylic acid

To a solution of ethyl (2R,3R)-1-((R)-tert-butylsulfinyl)-3-phenylaziridine-2-carboxylate (200 mg, 0.677 mmol) in THF (1.5 mL) at 0° C. was added a solution of LiOH (32.4 mg, 1.35 mmol) in H₂O (1.3 mL). The resulting mixture was stirred for 2 h at 0° C. and then acidified to pH 5 with 1M HCl. The aqueous layer was extracted with EtOAc (3×20 mL) and the combined organic layers were washed with brine (2×10 mL), dried with Na₂SO₄, filtered, and concentrated under reduced pressure to afford the desired compound (220 mg, crude) which was used without further purification. LCMS (ESI) m/z: [M+H] calcd for C₁₃H₁₇NO₃S: 268.10; found 268.4.

### Intermediate A-45 and A-46. Synthesis of and(S)—N-(1-(2-methoxyethyl) aziridine-2-carbonyl)-N-methylglycine and (R)—N-(1-(2-methoxyethyl) aziridine-2-carbonyl)-N-methylglycine

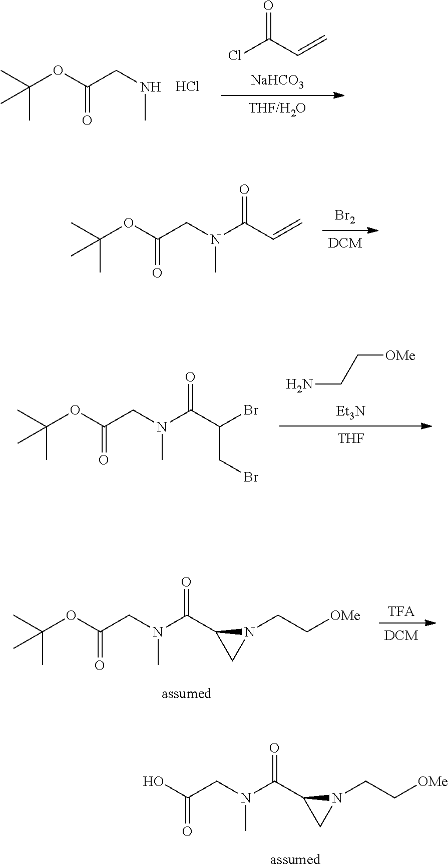

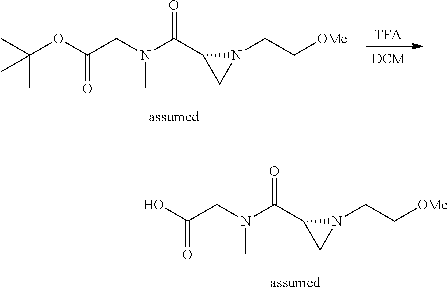

### Step 1: Synthesis of tert-butyl N-acryloyl-N-methylglycinate

To a mixture of tert-butyl methylglycinate hydrochloride (1.0 g, 5.5 mmol) and NaHCO₃ (1.39 g, 16.5 mmol) in THF (10 mL) and H₂O (5.0 mL) at 0° C. was added acryloyl chloride (750 mg, 8.26 mmol). The resulting solution was stirred for 2 h at room temperature and the reaction was then quenched by the addition H₂O (50 mL). The aqueous layer was extracted with EtOAc (2×50 mL) and the combined organic layers were washed with brine, dried with Na₂SO₄, filtered, and concentrated under reduced pressure. Purification by normal phase chromatography (10→33% EtOAc/pet. ether) afforded the desired product (900 mg, 73.8% yield). LCMS (ESI) m/z: [M+H] calcd for C₁₀H₁₇NO₃: 200.13; found 200.2.

### Step 2: Synthesis of tert-butyl N-(2,3-dibromopropanoyl)-N-methylglycinate

To a solution of tert-butyl N-acryloyl-N-methylglycinate (2.0 g, 10.1 mmol) in DCM (40 mL) at −20° C. was added Br₂ (3.21 g, 20.1 mmol). The resulting mixture was stirred for 2 h at −20° C. and then quenched by the addition of Na₂S₂O₃ (100 mL). The aqueous layer was extracted with DCM (2×100 mL) and the combined organic layers were washed with brine, dried with Na₂SO₄, and concentrated under reduced pressure to afford the desired product (2.4 g, crude) which was used without further purification. LCMS (ESI) m/z: [M+Na] calcd for C₁₀H₁₇Br₂NO₃: 381.96; found 381.8.

### Step 3: Synthesis of tert-butyl(S)—N-(1-(2-methoxyethyl) aziridine-2-carbonyl)-N-methylglycinate and tert-butyl (R)—N-(1-(2-methoxyethyl) aziridine-2-carbonyl)-N-methylglycinate

To a solution of tert-butyl N-(2,3-dibromopropanoyl)-N-methylglycinate (4.0 g, 11.1 mmol) and 2-methoxyethan-1-amine (4.18 g, 55.7 mmol) in THF (40 mL) was added Et₃N (4.66 mL, 33.4 mmol). The resulting solution was stirred at 35° C. overnight was then quenched by the addition of H₂O. The aqueous layer was extracted with DCM (2×100 mL) and the combined organic layers were washed with brine, dried with Na₂SO₄, filtered, and concentrated under reduced pressure. Purification by reverse phase chromatography (30→50% MeCN/H₂O) afforded a mixture of the desired products. The enantiomers were separated by chiral preparative normal phase chromatography (hexane, 10 mM NH₃-MeOH/EtOH) to afford tert-butyl(S)—N-(1-(2-methoxyethyl) aziridine-2-carbonyl)-N-methylglycinate (400 mg, 33.3% yield) and tert-butyl (R)—N-(1-(2-methoxyethyl) aziridine-2-carbonyl)-N-methylglycinate (360 mg, 30% yield). LCMS (ESI) m/z: [M+H] calcd for C₁₃H₂₄N₂O₄: 273.18; found 273.0.

### Step 4: Synthesis of(S)—N-(1-(2-methoxyethyl) aziridine-2-carbonyl)-N-methylglycine

To a solution of tert-butyl(S)—N-(1-(2-methoxyethyl) aziridine-2-carbonyl)-N-methylglycinate (250 mg, 0.918 mmol) in DCM (6.0 mL) at 0° C. was added TFA (3.0 mL). The resulting mixture was stirred at 2 h at 0° C. and then concentrated under reduced pressure to afford the desired product (250 mg, crude) which was used without further purification. LCMS (ESI) m/z: [M+H] calcd for C₉H₁₆N₂O₄: 217.12; found 217.1.

### Step 5: Synthesis of (R)—N-(1-(2-methoxyethyl) aziridine-2-carbonyl)-N-methylglycine

To a solution tert-butyl (R)—N-(1-(2-methoxyethyl) aziridine-2-carbonyl)-N-methylglycinate (180 mg, 0.661 mmol) in DCM (6.0 mL) at 0° C. was added TFA (3.0 mL). The resulting mixture was stirred at 2 h at 0° C. and then concentrated under reduced pressure to afford the desired product (150 mg, crude) which was used without further purification. LCMS (ESI) m/z: [M+H] calcd for C₉H₁₆N₂O₄: 217.12; found 217.1.

### Intermediate A-47 and A-48. Synthesis of N-methyl-N-(1-(((R)-1-methylaziridin-2-yl) sulfonyl) piperidine-4-carbonyl)-L-valine and N-methyl-N-(1-(((S)-1-methylaziridin-2-yl) sulfonyl) piperidine-4-carbonyl)-L-valine

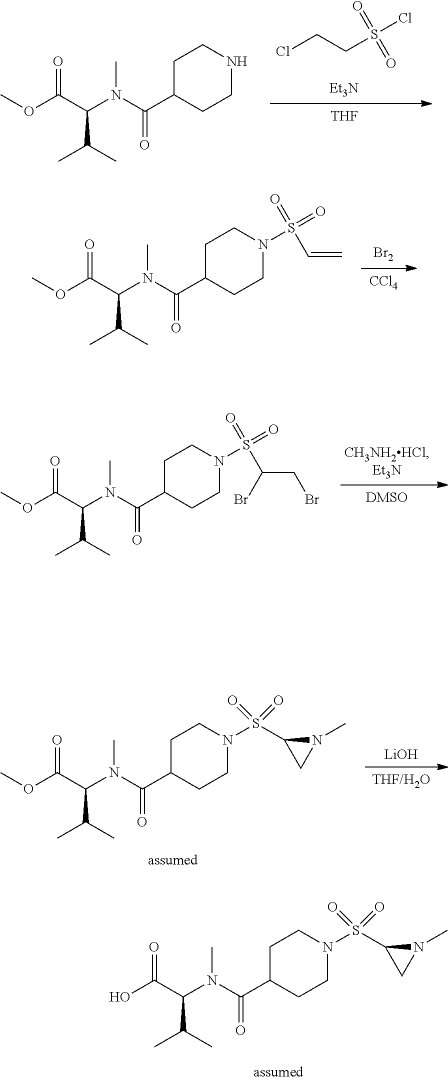

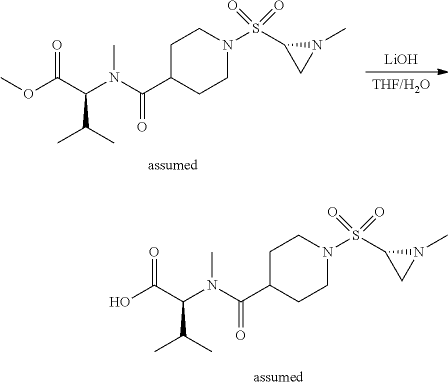

### Step 1: Synthesis of methyl N-methyl-N-(1-(vinylsulfonyl) piperidine-4-carbonyl)-L-valinate

To a solution of 2-chloroethanesulfonyl chloride (1.91 g, 11.7 mmol) in THF (20 mL) at −70° C. was added methyl N-methyl-N-(piperidine-4-carbonyl)-L-valinate (2.0 g, 7.8 mmol) followed by Et₃N (790, 780 mol). After warming to −50° C. additional Et₃N (790 IL, 780 mol) was added and the reaction mixture warmed to room temperature. After 1 h the reaction was quenched at 0° C. by the addition of H₂O (30 mL), acidified to pH 6 with 1M HCl, and extracted with CHCl₃ (3×30 mL). The combined organic layers were dried with MgSO₄, filtered, and concentrated under reduced pressure. Purification by normal phase chromatography (50% EtOAc/pet. ether) afforded the desired product (560 mg, 20.7% yield). LCMS (ESI) m/z: [M+H] calcd for C₁₅H₂₆N₂O₅S: 347.17; found 347.2.

### Step 2: Synthesis of methyl N-(1-((1,2-dibromoethyl) sulfonyl) piperidine-4-carbonyl)-N-methyl-L-valinate

To a solution of methyl N-methyl-N-(1-(vinylsulfonyl) piperidine-4-carbonyl)-L-valinate (580 mg, 1.67 mmol) in CCl4 (28 mL) at room temperature was added Br₂ (580 mg, 1.67 mmol) The resulting mixture was stirred overnight at room temperature and then quenched by the addition of sat. NaHCO₃ (30 mL). The aqueous layer was extracted with DCM (3×30 mL) and the combined organic layers were dried with Na₂SO₄, filtered and concentrated under reduced pressure to afford the desired product which was used without further purification. LCMS (ESI) m/z: [M+H] calcd for C₁₅H₂₆Br₂N₂O₅S: 506.99; found 506.9.

### Step 3: Synthesis of methyl N-methyl-N-(1-(((R)-1-methylaziridin-2-yl) sulfonyl) piperidine-4-carbonyl)-L-valinate and methyl N-methyl-N-(1-(((S)-1-methylaziridin-2-yl) sulfonyl) piperidine-4-carbonyl)-L-valinate

To a solution of methyl N-(1-((1,2-dibromoethyl) sulfonyl) piperidine-4-carbonyl)-N-methyl-L-valinate (4.80 g, 9.481 mmol) in DMSO (48 mL) was added methanamine hydrochloride (1.92 g, 28.436 mmol) and Et₃N (13.2 mL, 94.8 mmol). The reaction mixture was heated to 75° C. and stirred overnight. The mixture was then cooled to 0° C., diluted NH₄Cl, and extracted with EtOAc (600 mL). The organic layer was washed with brine, dried with Na₂SO₄, filtered, concentrated under reduced pressure. Purification with normal phase chromatography (86% EtOAc/hexane) afforded a mixture of the desired products. The diastereomers were separated by prep-SFC chromatography (20% IPA/CO₂) to afford methyl N-methyl-N-(1-(((R)-1-methylaziridin-2-yl) sulfonyl) piperidine-4-carbonyl)-L-valinate (700 mg, 38.9% yield) and methyl N-methyl-N-(1-(((S)-1-methylaziridin-2-yl) sulfonyl) piperidine-4-carbonyl)-L-valinate (790 mg, 43.9% yield). LCMS (ESI) m/z: [M+H] calcd for C₁₆H₂₉N₃O₅S: 376.19; found 376.1.

### Step 4: Synthesis of N-methyl-N-(1-(((R)-1-methylaziridin-2-yl) sulfonyl) piperidine-4-carbonyl)-L-valine

To a solution of methyl N-methyl-N-(1-(((R)-1-methylaziridin-2-yl) sulfonyl) piperidine-4-carbonyl)-L-valinate (200 mg, 0.533 mmol) in THF (2.0 mL) at 0° C. was added 1M LiOH (1 mL) The resulting mixture was stirred for 3 h at room temperature and then acidified to pH 6 with 1M HCl. The aqueous layer was extracted with EtOAc (3×10 mL) and the combined organic layers were dried with Na₂SO₄, filtered, and concentrated under reduced pressure to afford the desired product which was used without further purification. LCMS (ESI) m/z: [M+H] calcd for C₁₅H₂₇N₃O₅S: 362.18; found 362.2.

### Step 5: Synthesis of N-methyl-N-(1-(((S)-1-methylaziridin-2-yl) sulfonyl) piperidine-4-carbonyl)-L-valine

To a solution methyl N-methyl-N-(1-(((S)-1-methylaziridin-2-yl) sulfonyl) piperidine-4-carbonyl)-L-valinate (300 mg, 0.799 mmol) in THF (3.0 mL) at 0° C. was added 1M LiOH (3.0 mL). The resulting mixture was stirred for 3 h at room temperature and then acidified to pH 6 with 1M HCl. The aqueous layer was extracted with EtOAc (3×10 mL) and the combined organic layers dried with Na₂SO₄, filtered and concentrated under reduced pressure to afford the desired product which was used without further purification. LCMS (ESI) m/z: [M+H] calcd for C₁₅H₂₇N₃O₅S: 362.18; found 362.2.

### Intermediate A-49 and A-50. Synthesis of N-methyl-N-(1-(((R)-1-methylaziridin-2-yl) sulfonyl) azetidine-3-carbonyl)-L-valine and N-methyl-N-(1-(((S)-1-methylaziridin-2-yl) sulfonyl) azetidine-3-carbonyl)-L-valine

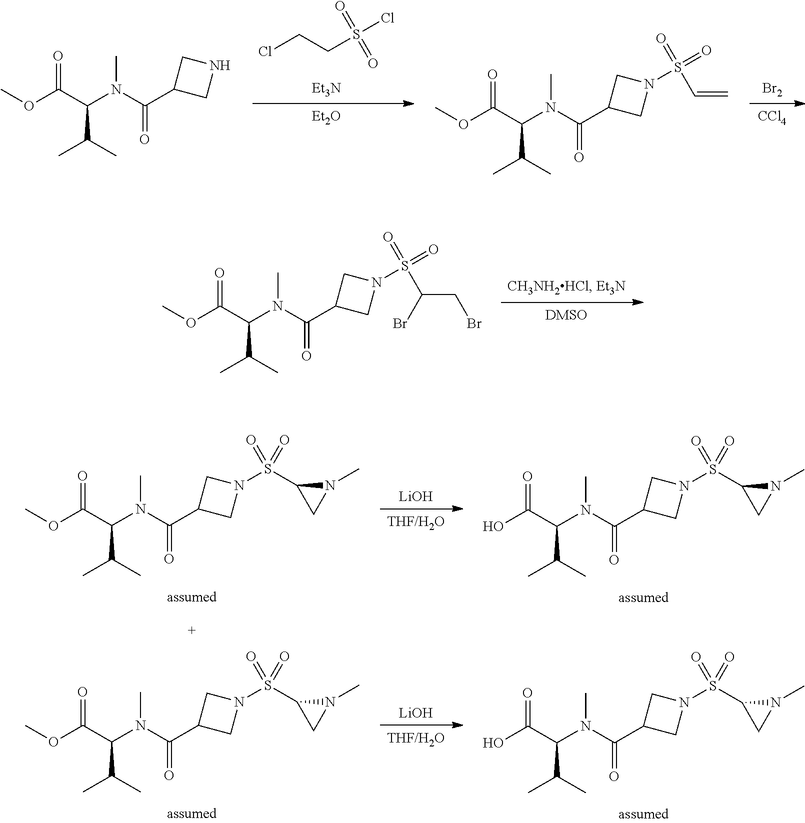

### Step 1: Synthesis of methyl N-methyl-N-(1-(vinylsulfonyl) azetidine-3-carbonyl)-L-valinate

To a solution of 2-chloroethanesulfonyl chloride (357 mg, 2.19 mmol) in Et₂O (4.0 mL) at-70° C. was added an Et₂O (4.0 mL) solution of methyl N-(azetidine-3-carbonyl)-N-methyl-L-valinate (500 mg, 2.19 mmol) followed by Et₃N (0.304 mL, 2.19 mmol). The resulting mixture was stirred for 30 min at −50° C. at which time Et₃N (0.304 mL, 2.19 mmol) was added. The resulting mixture was stirred for 1 h at room temperature and then quenched with H₂O at 0° C. The mixture was acidified to pH 6 with 1M HCl and extracted with CHCl₃ (3×10 mL). The combined organic layers were washed with brine, dried with Na₂SO₄, filtered, and concentrated under reduced pressure. Purification by normal phase chromatography (50% EtOAc/pet. ether) afforded the desired product (180 mg, 25.8% yield). LCMS (ESI) m/z: [M+H] calcd for C₁₃H₂₂N₂O₅S: 319.13; found 319.1.

### Step 2: Synthesis of methyl N-(1-((1,2-dibromoethyl) sulfonyl) azetidine-3-carbonyl)-N-methyl-L-valinate

To a solution of methyl N-methyl-N-(1-(vinylsulfonyl) azetidine-3-carbonyl)-L-valinate (460 mg, 1.45 mmol) in CCl₄ (6.0 mL) at room temperature was added a CCl₄ (2.0 mL) solution of Br₂ (346 mg, 2.17 mmol). The resulting mixture was stirred overnight and then quenched at 0° C. by the addition of sat. NaHCO₃ and Na₂S₂O₃. The aqueous layer was extracted with DCM (3×10 mL) and the combined organic layers were washed with brine, dried with Na₂SO₄, and concentrated under reduced pressure to afford the desired product (500 mg) which was used without further purification. LCMS (ESI) m/z: [M+H] calcd for C₁₃H₂₂Br₂N₂O₅S: 478.97; found 478.0.

### Step 3: Synthesis of methyl N-methyl-N-(1-(((R)-1-methylaziridin-2-yl) sulfonyl) azetidine-3-carbonyl)-L-valinate and methyl N-methyl-N-(1-(((S)-1-methylaziridin-2-yl) sulfonyl) azetidine-3-carbonyl)-L-valinate

To a solution of methyl N-(1-((1,2-dibromoethyl) sulfonyl) azetidine-3-carbonyl)-N-methyl-L-valinate (260 mg, 0.54 mmol) in DMSO (4.0 mL) was added methanamine hydrochloride (110.0 mg, 1.63 mmol) and Et₃N (0.758 mL, 5.44 mmol). The resulting mixture was heated to 75° C. and stirred overnight. The mixture was then cooled to room temperature, diluted with H₂O (10 mL), and extracted with EtOAc (3×5 mL). The combined organic layers were washed with brine, dried with Na₂SO₄, filtered and concentrated under reduced pressure. Purification by normal phase chromatography (50% EtOAc/pet. ether) afforded a mixture of the desired products. The diastereomers were separated by chiral prep normal phase chromatography (hexane, 10 mM NH₃-MeOH/IPA) to afford methyl N-methyl-N-(1-(((R)-1-methylaziridin-2-yl) sulfonyl) azetidine-3-carbonyl)-L-valinate (0.59 g, 35% yield) and methyl N-methyl-N-(1-(((S)-1-methylaziridin-2-yl) sulfonyl) azetidine-3-carbonyl)-L-valinate (0.56 g, 33% yield). LCMS (ESI) m/z: [M+H] calcd for C₁₄H₂₅N₃O₅S: 348.16; found 348.2.

### Step 4: Synthesis of N-methyl-N-(1-(((R)-1-methylaziridin-2-yl) sulfonyl) azetidine-3-carbonyl)-L-valine

To a solution of methyl N-methyl-N-(1-(((R)-1-methylaziridin-2-yl) sulfonyl) azetidine-3-carbonyl)-L-valinate (225.0 mg, 0.65 mmol) in THF (1.5 mL) at 0° C. was added LiOH (77.0 mg, 3.23 mmol) dissolved in H₂O (1.5 mL). The resulting mixture was stirred for 2 h at room temperature and then acidified to pH 6 with 1M HCl. The aqueous layer was extracted with EtOAc and the combined organic layers were washed with brine, dried with Na₂SO₄, filtered, and concentrated under reduced pressure to afford the desired product (270 mg) which was used without further purification. LCMS (ESI) m/z: [M+H] calcd for C₁₃H₂₃N₃O₅S: 334.15; found 334.0.

### Step 5: Synthesis of N-methyl-N-(1-(((S)-1-methylaziridin-2-yl) sulfonyl) azetidine-3-carbonyl)-L-valine

To a solution of methyl N-methyl-N-(1-(((S)-1-methylaziridin-2-yl) sulfonyl) azetidine-3-carbonyl)-L-valinate (365.0 mg, 1.05 mmol) in THF (2.0 mL) and H₂O (2.0 mL) at 0° C. was added LiOH hydrate (132.0 mg, 3.15 mmol). The resulting mixture was stirred for 2 h at room temperature then acidified to pH 6 with 1M HCl and diluted with H₂O (20 mL). The aqueous layer was extracted with EtOAc and the combined organic layers were washed with brine, dried with Na₂SO₄, filtered, and concentrated under reduced pressure to afford the desired product (257 mg, crude) which was used without further purification. LCMS (ESI) m/z: [M+H] calcd for C₁₃H₂₃N₃O₅S: 334.15; found 334.3.

### Intermediate A-51. Synthesis of 2-((1R,5S)-2,4-dioxo-6-trityl-3,6-diazabicyclo[3.1.0]hexan-3-yl) acetic acid

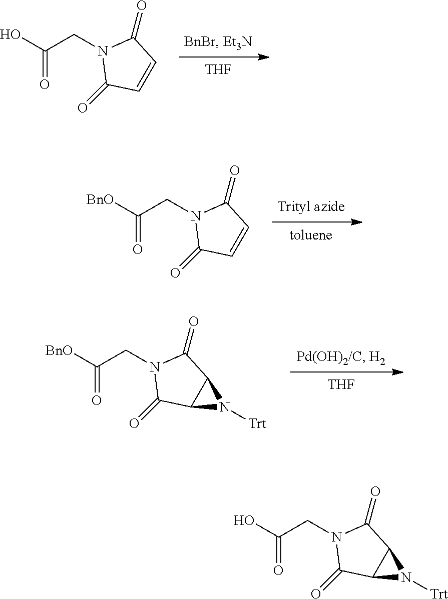

### Step 1: Synthesis of benzyl 2-(2,5-dioxo-2,5-dihydro-1H-pyrrol-1-yl) acetate

To a solution of 2-(2,5-dioxo-2,5-dihydro-1H-pyrrol-1-yl) acetic acid (5.0 g, 32.2 mmol) and Et₃N (13.5 mL, 96.7 mmol) in THF (80 mL) at 0° C. was added benzyl bromide (11.03 g, 64.5 mmol). The resulting mixture was stirred overnight at room temperature and then filtered. The filter cake was washed with THF (3×40 mL) and the filtrate was concentrated under reduced pressure. Purification by silica gel chromatography (16% EtOAc/hexanes) afforded the desired product (4.4 g, 55.7% yield) LCMS (ESI) m/z: [2M+Na] calcd for C₁₃H₁₁NO₄: 513.14; found 513.2.

### Step 2: Synthesis of benzyl 2-((1R,5S)-2,4-dioxo-6-trityl-3,6-diazabicyclo[3.1.0]hexan-3-yl) acetate

To a solution of benzyl 2-(2,5-dioxo-2,5-dihydro-1H-pyrrol-1-yl) acetate of (1.0 g, 4.0 mmol) in toluene (10 mL) was added trityl azide (1.36 g, 4.89 mmol). The resulting mixture was stirred overnight at 120° C. and then concentrated under reduced pressure. Purification by reverse flash chromatography (50→80% MeCN/H₂O) afforded the desired product (400 mg, 19.5% yield). LCMS (ESI) m/z: [M+Na] calcd for C₃₂H₂₆N₂O₄: 525.19; found 525.2.

### Step 3: Synthesis of 2-((1R,5S)-2,4-dioxo-6-trityl-3,6-diazabicyclo[3.1.0]hexan-3-yl) acetic acid

To a solution of benzyl 2-((1R,5S)-2,4-dioxo-6-trityl-3,6-diazabicyclo[3.1.0]hexan-3-yl) acetate (220 mg, 0.438 mmol) in THF (8.0 mL) was added Pd (OH) 2/C(60 mg). The resulting solution was placed under a hydrogen atmosphere for 3 h using a H₂ balloon, filtered through Celite, and concentrated under reduced pressure to afford the desired product (160 mg, crude) which was used without further purification. LCMS (ESI) m/z: [M−H] calcd for C₂₅H₂₀N₂O₄: 411.13; found 411.2.

### Intermediate A-52. Synthesis of(S)-3-methyl-2-(5-oxo-2-((S)-1-tritylaziridine-2-carbonyl)-2,6-diazaspiro[3.4]octan-6-yl) butanoic acid

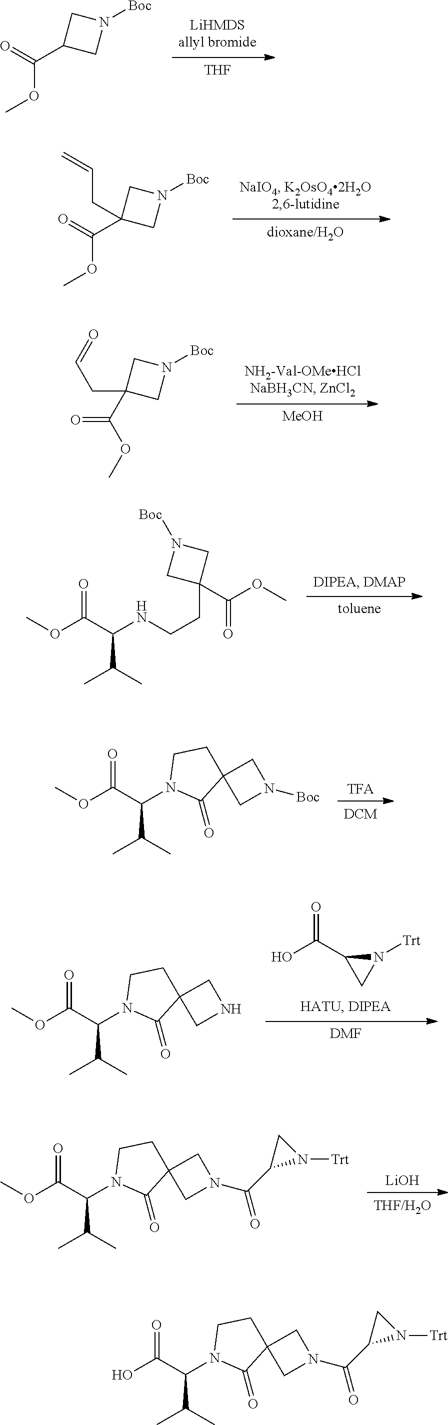

### Step 1: Synthesis of 1-(tert-butyl) 3-methyl 3-allylazetidine-1,3-dicarboxylate

To a solution of 1-(tert-butyl) 3-methyl azetidine-1,3-dicarboxylate (20.0 g, 92.9 mmol) and LiHMDS (140 mL, 1M in THF, 139 mmol) in THF (200 mL) at −78° C. was added allyl bromide (16.9 g, 139 mmol). The resulting solution was stirred overnight at room temperature and then quenched with the addition of sat. NH₄Cl (100 mL) and diluted with EtOAc (800 mL). The organic layer was washed with brine (3×300 mL), dried with Na₂SO₄, filtered and concentrated under reduced pressure. Purification by silica gel column chromatography (17% EtOAc/pet. ether) afforded the desired product (15.0 g, 63.2% yield). LCMS (ESI) m/z: [M+H-tBu] calcd for C₁₃H₂₁NO₄: 200.10; found 200.0 .

### Step 2: Synthesis of 1-(tert-butyl) 3-methyl 3-(2-oxoethyl) azetidine-1,3-dicarboxylate

To a solution of 1-(tert-butyl) 3-methyl 3-allylazetidine-1,3-dicarboxylate (6.0 g, 23 mmol) and 2,6-lutidine (504 mg, 47.0 mmol) in dioxane (60 mL) and H₂O (60 mL) at 0° C. was added K₂OsO₄·2H₂O (433 mg, 1.18 mmol). The resulting mixture was stirred at room temperature for 15 min then NaIO₄ (20.1 g, 94.0 mmol) was added at 0° C. The reaction was stirred for 3 h at room temperature and then quenched with sat. Na₂S₂O₃ at 0° C. The aqueous layer was extracted with EtOAc (2×400 mL) and the combined organic layers were washed with 1 M HCl (2×80 mL), brine (2×100 mL), dried with Na₂SO₄, filtered and concentrated under reduced pressure to afford the desired product (2.84 g, crude) which was used without further purification. LCMS (ESI) m/z: [M−H] calcd for C₁₂H₁₉NO₅: 256.12; found 256.0 .

### Step 3: Synthesis of 1-(tert-butyl) 3-methyl(S)-3-(2-((1-methoxy-3-methyl-1-oxobutan-2-yl) amino) ethyl) azetidine-1,3-dicarboxylate

To a solution of 1-(tert-butyl) 3-methyl 3-(2-oxoethyl) azetidine-1,3-dicarboxylate (13.0 g, 50.5 mmol) and methyl L-valinate hydrochloride (7.29 g, 55.6 mmol) in MeOH (130 mL) at 0° C. were added ZnCl₂ (7.57 g, 55.6 mmol) and NaBH₃CN (6.35 g, 101 mmol). The resulting mixture was stirred at room temperature overnight, partially concentrated under reduced pressure and diluted with EtOAc (500 mL). The resulting solution was washed with brine (3×200 mL), dried with Na₂SO₄, filtered, and concentrated under reduced pressure. Purification by normal phase chromatography (10→66% EtOAc/pet. ether) afforded the desired product (7.72 g, 41.0% yield). LCMS (ESI) m/z: [M+H] calcd for C₁₈H₃₂N₂O₆: 373.24; found 373.1.

### Step 4: Synthesis of tert-butyl(S)-6-(1-methoxy-3-methyl-1-oxobutan-2-yl)-5-oxo-2,6-diazaspiro[3.4]octane-2-carboxylate

To a solution of 1-(tert-butyl) 3-methyl(S)-3-(2-((1-methoxy-3-methyl-1-oxobutan-2-yl) amino) ethyl) azetidine-1,3-dicarboxylate (6.0 g, 16 mmol) and DIPEA (28.0 mL, 161 mmol) in toluene (60 mL) at room temperature was added DMAP (197 mg, 1.61 mmol). The resulting mixture was stirred at 80° C. overnight, diluted with EtOAc (50 mL), washed with H₂O (50 mL), brine (3×50 mL) dried with Na₂SO₄, and filtered, and concentrated under reduced pressure. Purification by reverse phase chromatography 45→80% MeCN/H₂O) afforded the desired product (4.3 g, 78.4% yield). LCMS (ESI) m/z: [M+H-^{t}Bu] calcd for C₁₇H₂₈N₂O₅: 285.15; found 285.0 .

### Step 5: Synthesis of methyl(S)-3-methyl-2-(5-oxo-2,6-diazaspiro[3.4]octan-6-yl) butanoate

To a solution of tert-butyl(S)-6-(1-methoxy-3-methyl-1-oxobutan-2-yl)-5-oxo-2,6-diazaspiro[3.4]octane-2-carboxylate (2.7 g, 7.9 mmol) in DCM (27 mL) at room temperature was added TFA (8.10 mL, 71.0 mmol). The resulting mixture was stirred for 1 h and then concentrated under reduced pressure to afford the desired product, (1.70 g, crude) which was used without further purification. LCMS (ESI) m/z: [M+H] calcd for C₁₂H₂₀N₂O₃: 241.16; found 240.1.

### Step 6: Synthesis of methyl(S)-3-methyl-2-(5-oxo-2-((S)-1-tritylaziridine-2-carbonyl)-2,6-diazaspiro[3.4]octan-6-yl) butanoate

To a solution methyl(S)-3-methyl-2-(5-oxo-2,6-diazaspiro[3.4]octan-6-yl) butanoate (700 mg, 2.91 mmol) and(S)-1-tritylaziridine-2-carboxylic acid (1.15 g, 3.50 mmol) in DMF (7.0 mL) at 0° C. was added DIPEA (2.5 mL, 14.6 mmol). After 30 min HATU (1.66 g, 4.37 mmol) was added and the resulting mixture was stirred at room temperature for 1 h. The reaction was then diluted with EtOAc (20 mL) and the organic layer was washed with sat. NH₄Cl (50 mL), brine (3×50 mL), dried with Na₂SO₄, filtered, and concentrated under reduced pressure. Purification by normal phase chromatography (0→80% EtOAc/pet. ether) afforded the desired product (300 mg, 18.7% yield).

LCMS (ESI) m/z: [M+H] calcd for C₃₄H₃₇N₃O₄: 552.29; found 552.2.

### Step 7: Synthesis of(S)-3-methyl-2-(5-oxo-2-((S)-1-tritylaziridine-2-carbonyl)-2,6-diazaspiro[3.4]octan-6-yl) butanoic acid

To a solution of methyl(S)-3-methyl-2-(5-oxo-2-((S)-1-tritylaziridine-2-carbonyl)-2,6-diazaspiro[3.4]octan-6-yl) butanoate (700 mg, 1.27 mmol) in THF (10 mL) and H₂O (2.0 mL) at 0° C. was added LiOH (152 mg, 6.34 mmol). After 30 min the reaction mixture was warmed to room temperature for 1 h and then acidified to pH 6 with 1M HCl. The aqueous layer was extracted with EtOAc (3×50 mL) and the combined organic layers were washed with brine, dried with Na₂SO₄, filtered, and concentrated under reduced pressure to afford the desired product (300 mg, 18.7% yield) which was used without further purification. LCMS (ESI) m/z: [M+H] calcd for C₃₃H₃₅N₃O₄: 538.27; found 538.2.

### Intermediate A-53. Synthesis of(S)-3-methyl-2-(5-oxo-2-((R)-1-tritylaziridine-2-carbonyl)-2,6-diazaspiro[3.4]octan-6-yl) butanoic acid

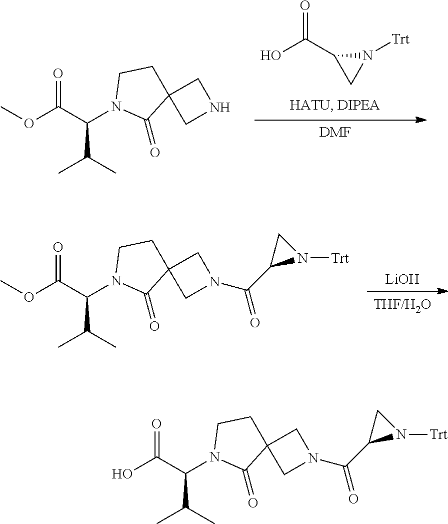

### Step 1: Synthesis of methyl(S)-3-methyl-2-(5-oxo-2-((R)-1-tritylaziridine-2-carbonyl)-2,6-diazaspiro[3.4]octan-6-yl) butanoate

To a solution (R)-1-tritylaziridine-2-carboxylic acid (1.0 g, 3.0 mmol) and methyl(S)-3-methyl-2-(5-oxo-2,6-diazaspiro[3.4]octan-6-yl) butanoate (875 mg, 3.64 mmol) in DMF (10 mL) at 0° C. was added DIPEA (2.64 mL, 15.2 mmol). After 30 min HATU (1.73 g, 4.554 mmol) was added and the resulting mixture was stirred for 1 h at room temperature. The reaction was then diluted with EtOAc (20 mL) and the organic layer was washed with sat. NH₄Cl (50 mL), brine (3×50 mL), dried with Na₂SO₄, filtered, and concentrated under reduced pressure. Purification by silica gel chromatography (0→80% EtOAc/pet. ether) afforded the desired product (789 mg, 47% yield). LCMS (ESI) m/z: [M+H] calcd for C₃₄H₃₇N₃O₄: 552.29; found 552.3.

### Step 2: Synthesis of(S)-3-methyl-2-(5-oxo-2-((R)-1-tritylaziridine-2-carbonyl)-2,6-diazaspiro[3.4]octan-6-yl) butanoic acid

To a stirred solution of methyl(S)-3-methyl-2-(5-oxo-2-((R)-1-tritylaziridine-2-carbonyl)-2,6-diazaspiro[3.4]octan-6-yl) butanoate (900 mg, 1.63 mmol) in THF (10 mL) and H₂O (2.5 mL) at 0° C. was added LiOH (156 mg, 6.53 mmol). After 30 min the reaction mixture was warmed to room temperature for 1 h and then acidified to pH 6 with 1M HCl. The aqueous layer was extracted with EtOAc (3×50 mL) and the combined organic layers were washed with brine, dried with Na₂SO₄, filtered, and concentrated under reduced pressure to afford the desired product (240 mg, 27.4% yield). LCMS (ESI) m/z: [M+H] calcd for C₃₃H₃₅N₃O₄: 538.27; found 538.2.

### Intermediate A-54. Synthesis of(S)-1-((R)-2-(methoxycarbonyl) aziridine-1-carbonyl) pyrrolidine-3-carboxylic acid

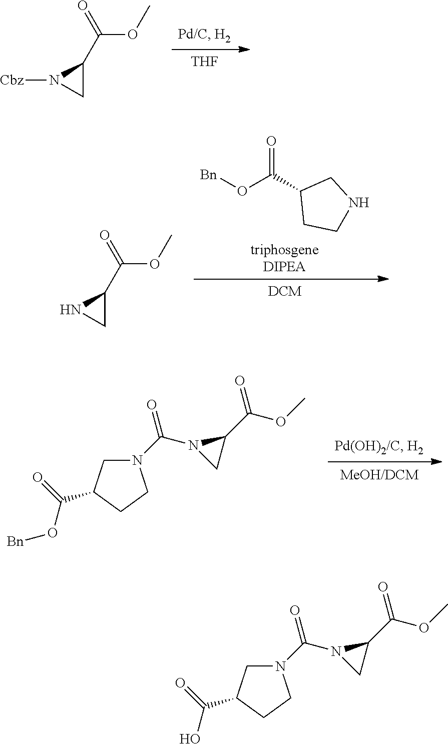

### Step 1: Synthesis of methyl (R)-aziridine-2-carboxylate

A suspension of 1-benzyl 2-methyl (R)-aziridine-1,2-dicarboxylate (1.50 g, 6.4 mmol) and Pd/C (300 mg, 2.8 mmol) in THF (15 mL) under an atmosphere of hydrogen (1 atm) was stirred for 3 h before the solids were removed by filtration. The crude solution was concentrated under reduced pressure which afforded desired product (600 mg, crude). LCMS (ESI) m/z: [M+H] calcd for C₄H₇NO₂: 102.06; found 102.3.

### Step 2: Synthesis of benzyl(S)-1-((R)-2-(methoxycarbonyl) aziridine-1-carbonyl) pyrrolidine-3-carboxylate

To solution of methyl (R)-aziridine-2-carboxylate (1.0 g, 9.90 mmol) and benzyl(S)-pyrrolidine-3-carboxylate (2.63 g, 10.9 mmol, HCl salt) in DCM (30.0 mL) at −10° C. was added DIPEA (10.3 mL, 59.3 mmol) followed by triphosgene (880 mg, 2.97 mmol). The resulting solution was stirred for 30 min and was then quenched by the addition of H₂O (50 mL). The aqueous layer was extracted with DCM (2×100 mL), washed with brine (2×50 mL), dried over Na₂SO₄, and concentrated under reduced pressure. Purification by prep-TLC(50% EtOAc/pet. ether) afforded desired product (1.30 g, 28% yield). LCMS (ESI) m/z: [M+H] calcd for C₁₇H₂₀N₂O₅: 333.15; found 333.2.

### Step 3: Synthesis of(S)-1-((R)-2-(methoxycarbonyl) aziridine-1-carbonyl) pyrrolidine-3-carboxylic acid

To a solution of benzyl(S)-1-((R)-2-(methoxycarbonyl) aziridine-1-carbonyl) pyrrolidine-3-carboxylate (200 mg, 600 μmol) in MeOH (5 mL) and DCM (5 mL) under H₂ was added Pd (OH) 2/C (130 mg, 90 μmol). The resulting mixture was stirred for 30 min at room temperature and then the mixture was filtered. The filter cake was washed with MeOH (2×10 mL) and the filtrate was concentrated under reduced pressure which afforded desired product (140 mg, 96% yield) as an off-white solid. LCMS (ESI) m/z: [M+H] calcd for C₁₀H₁₄N₂O₅: 243.10; found 243.3.

### Intermediate A-55. Synthesis of(S)-1-((S)-2-(methoxycarbonyl) aziridine-1-carbonyl) pyrrolidine-3-carboxylic acid

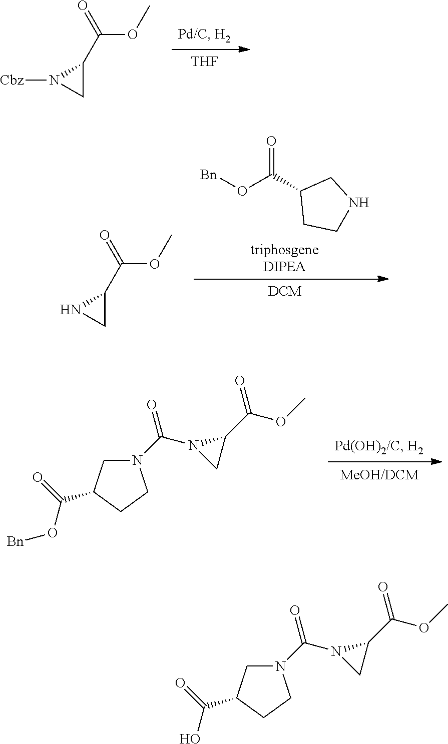

### Step 1: Synthesis of methyl(S)-aziridine-2-carboxylate

A suspension of 1-benzyl 2-methyl(S)-aziridine-1,2-dicarboxylate (200 mg, 850 μmol) and Pd/C (20 mg, 38 μmol) in THF (4.0 mL) under an atmosphere of hydrogen (1 atm) was stirred for 2 h before the solids were removed by filtration. The crude solution was concentrated under reduced pressure which afforded desired product (92 mg, crude). LCMS (ESI) m/z: [M+H] calcd for C₄H₇NO₂: 102.06; found 102.3.

### Step 2: Synthesis of benzyl(S)-1-((S)-2-(methoxycarbonyl) aziridine-1-carbonyl) pyrrolidine-3-carboxylate

To a solution of methyl(S)-aziridine-2-carboxylate (900 mg, 8.9 mmol) and benzyl(S)-pyrrolidine-3-carboxylate (2.37 g, 9.80 mmol, HCl salt) in DCM (30 mL) at −10° C. was added DIPEA (9.30 mL, 53.4 mmol) followed by triphosgene (790 mg, 2.67 mmol). The resulting solution was stirred for 30 min and was then quenched by the addition of H₂O (50 mL). The aqueous layer was extracted with DCM (2×100 mL), washed with brine (2×50 mL), dried over Na₂SO₄, filtered, and concentrated under reduced pressure. Purification by prep-TLC(50% EtOAc/pet. ether) afforded desired product (360 mg, 8.5% yield) as an off-white oil. LCMS (ESI) m/z: [M+H] calcd for C₁₇H₂₀N₂O₅: 333.15; found 333.2.

### Step 3: Synthesis of(S)-1-((S)-2-(methoxycarbonyl) aziridine-1-carbonyl) pyrrolidine-3-carboxylic acid

To a solution of benzyl(S)-1-((S)-2-(methoxycarbonyl) aziridine-1-carbonyl) pyrrolidine-3-carboxylate (130 mg, 390 μmol) in MeOH (3 mL) and DCM (3 mL) under H₂ was added Pd (OH) 2/C (55 mg, 39 μmol). The resulting solution was stirred for 30 min at room temperature and then the reaction mixture was filtered. The filter cake was washed with MeOH (2×10 mL) and the filtrate was concentrated under reduced pressure which afforded desired product (90 mg, 95% yield) as an off-white solid. LCMS (ESI) m/z: [M+H] calcd for C₁₀H₁₄N₂O₅: 243.10; found 243.3.

### Intermediate A-56. Synthesis of (2R,3S)-3-cyclopropylaziridine-2-carboxylic acid

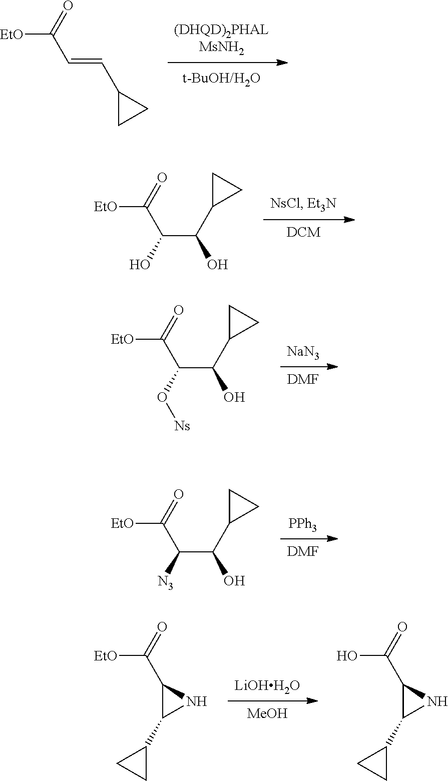

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD)₂PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAc/pet. ether) afforded desired product (5.5 g, 44% yield).

### Step 2: Synthesis of ethyl (2S,3R)-3-cyclopropyl-3-hydroxy-2-(((4-nitrophenyl) sulfonyl)oxy) propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenyl) sulfonyl)oxy) propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH·H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

### Intermediate A-57. Synthesis of (2R,3S)-3-cyclopropylaziridine-2-carboxylic acid

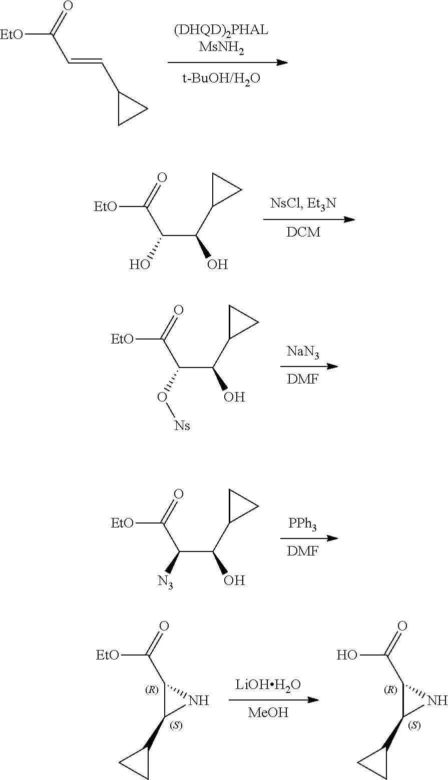

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD) 2PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAC/pet. ether) afforded desired product (5.5 g, 44% yield).

### Step 2: Synthesis of ethyl (2S,3R)-3-cyclopropyl-3-hydroxy-2-(((4-nitrophenyl) sulfonyl)oxy) propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenyl) sulfonyl)oxy) propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH·H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

### Intermediate A-58. Synthesis of (2S,3R)-3-cyclopropylaziridine-2-carboxylic acid

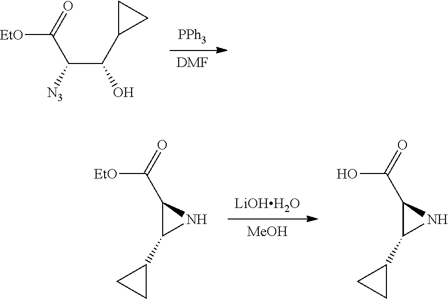

### Step 1: Synthesis of ethyl (2S,3R)-3-cyclopropylaziridine-2-carboxylate

A mixture of PPh₃ (1.4 g, 5.4 mmol) in DMF (15.0 mL) was stirred at 0° C. After 30 min, ethyl (2S,3S)-2-azido-3-cyclopropyl-3-hydroxypropanoate (980 mg, 4.92 mmol) was added. The reaction mixture was heated to 80° C. After 2 h the reaction was quenched by the addition of H₂O (20 mL) and was extracted with EtOAc (3×30 mL). Purification by silica gel column chromatography (17% EtOAc/pet. ether) afforded desired product (500 mg, 65% yield).

### Step 2: Synthesis of lithium (2S,3R)-3-cyclopropylaziridine-2-carboxylate

To a solution of ethyl (2S,3R)-3-cyclopropylaziridine-2-carboxylate (450 mg, 2.9 mmol) in THF (6.0 mL) and H₂O (2.0 mL) was added LiOH (90 mg, 3.8 mmol). The reaction was stirred for 2 h and then filtered. The filtrate was concentrated under reduced pressure which afforded the desired product (300 mg, crude).

### Intermediate A-59. Synthesis of (R)-3-methyl-2-(((R)—N-methyl-1-tritylaziridine-2-carboxamido) methyl) butanoic acid

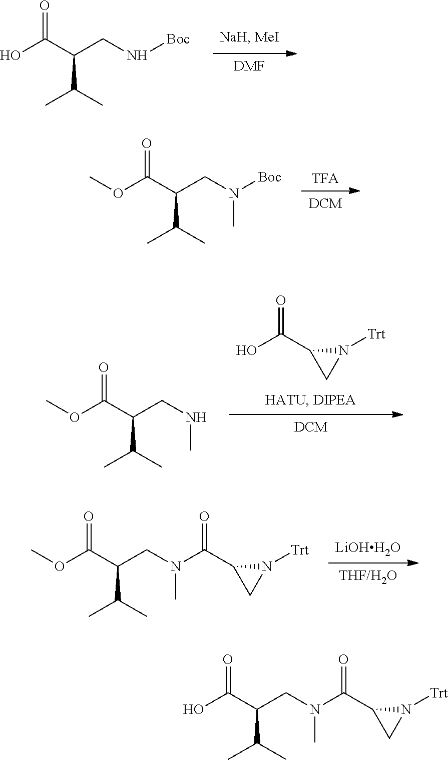

### Step 1: Synthesis of methyl (R)-2-(((tert-butoxycarbonyl) (methyl) amino) methyl)-3-methylbutanoate

To a solution of (R)-2-(((tert-butoxycarbonyl) amino) methyl)-3-methylbutanoic acid (500 mg, 2.16 mmol) in DMF (10.0 mL) at 0° C. was added NaH (130 mg, 5.40 mmol). After 30 min, Mel (540 μL, 8.65 mmol) was added and the reaction was warmed to room temperature. After 2 h the reaction was cooled to 0° C. and quenched by the addition of sat. aq. NH₄Cl (10 mL). The resulting mixture was extracted with EtOAc (3×20 mL) and the combined organic layers were washed with brine (40 mL), dried with Na₂SO₄, and concentrated under reduced pressure. Purification by prep-TLC(9% EtOAc/pet. ether) afforded the desired product (500 mg, 89.2% yield). LCMS (ESI) m/z: [M+H] calcd for C₁₃H₂₅NO₄: 260.19; found 260.2.

### Step 2: Synthesis of methyl (R)-3-methyl-2-((methylamino) methyl) butanoate

To a solution of methyl (R)-2-(((tert-butoxycarbonyl) (methyl) amino) methyl)-3-methylbutanoate (500 mg, 1.93 mmol) in DCM (5.0 mL) at 0° C. was added TFA (2.50 mL) dropwise. The resulting mixture was warmed to room temperature. After 2 h the reaction mixture was concentrated under reduced pressure to afford desired product (600 mg, crude) as a yellow solid.

### Step 3: Synthesis of methyl (R)-3-methyl-2-(((R)—N-methyl-1-tritylaziridine-2-carboxamido) methyl) butanoate

To a solution of methyl (R)-3-methyl-2-((methylamino) methyl) butanoate (550 mg, 3.45 mmol) and (R)-1-tritylaziridine-2-carboxylic acid (1.25 g, 3.80 mmol) in DCM (5.0 mL) at 0° C. was added DIPEA (1.81 mL, 10.4 mmol) followed by HATU (1.58 g, 4.15 mmol). The resulting mixture was warmed to room temperature. After 2 h the reaction was quenched with H₂O (30 mL) and extracted with EtOAc (3×30 mL). The combined organic layers were washed with brine (50 mL), dried with Na₂SO₄, and concentrated under reduced pressure. Purification by silica gel column chromatography (9% EtOAc/pet. ether) afforded the desired product (300 mg, 19% yield) as a yellow oil. LCMS (ESI) m/z: [M+H] calcd for C₃₀H₃₄N₂O₃: 471.27; found 471.3.

### Step 4: Synthesis of (R)-3-methyl-2-(((R)—N-methyl-1-tritylaziridine-2-carboxamido) methyl) butanoic acid

To a solution of methyl (R)-3-methyl-2-(((R)—N-methyl-1-tritylaziridine-2-carboxamido) methyl) butanoate (200 mg, 0.425 mmol) in THF (2.0 mL) at 0° C. was added LiOH·H₂O (89 mg, 2.13 mmol) in H₂O (2.0 mL) dropwise. The resulting mixture was warmed to room temperature. After 5 h the mixture was neutralized to pH 7 with 1M HCl. The reaction was extracted with EtOAc (3×20 mL). The combined organic layers were washed with brine (30 mL), dried with Na₂SO₄, and concentrated under reduced pressure to afford product (200 mg, crude) as an off-white solid. The crude product was used in the next step directly without further purification. LCMS (ESI) m/z: [M+H] calcd for C₂₉H₃₂N₂O₃: 457.25; found 457.2.

### Intermediate A-60. Synthesis of sodium (R)-1-methyl-5-(1-tritylaziridine-2-carboxamido)-1H-imidazole-2-carboxylate

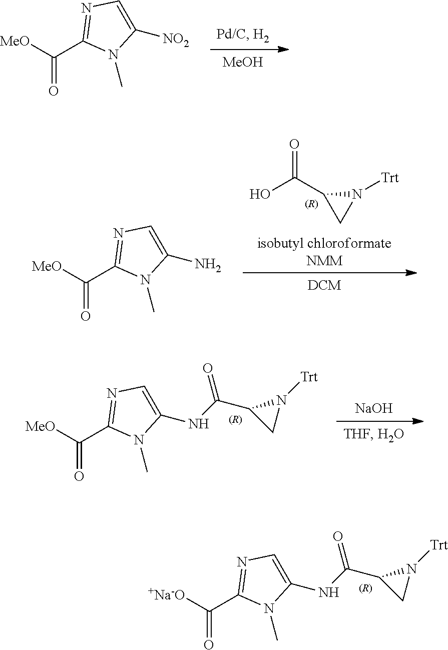

### Step 1: Synthesis of methyl 5-amino-1-methyl-1H-imidazole-2-carboxylate

A mixture of methyl 1-methyl-5-nitro-1H-imidazole-2-carboxylate (1.0 g, 5.401 mmol) and Pd/C (500.0 mg) in MeOH (15 mL) at room temperature was stirred under an atmosphere of hydrogen (1 atm) for 3 h. The mixture was filtered and the filter cake was washed with MeOH (3×20 mL). The filtrate was concentrated under reduced pressure to afford the desired product (1.0 g, crude). LCMS (ESI) m/z: [M+Na] calcd for C₆H₉N₃O₂: 156.08; found 156.1.

### Step 2: Synthesis of methyl (R)-1-methyl-5-(1-tritylaziridine-2-carboxamido)-1-imidazole-2-carboxylate

A solution of (R)-1-tritylaziridine-2-carboxylic acid (2.55 g, 7.741 mmol) in DCM (12.0 mL) at 0° C. was added in portions over 30 min to a solution of isobutyl chloroformate (845.1 mg, 6.187 mmol) and N-methylmorpholine (1.04 g, 10.282 mmol) in DCM. To the mixture was then added methyl 5-amino-1-methyl-1H-imidazole-2-carboxylate (800.0 mg, 5.156 mmol) at 0° C. The resulting mixture was warmed to room temperature and stirred overnight. The mixture was diluted with DCM (300 mL) and washed with H₂O (3×100 mL), washed with brine (2×150 mL), dried over Na₂SO₄, filtered, and concentrated under reduced pressure. The residue was purified by silica gel chromatography (25% EtOAc/hexanes) to afford the final product (1.2 g, 49.9% yield). LCMS (ESI) m/z: [M+H] calcd for C₂₈H₂₆N₄O₃: 467.21; found 467.2.

### Step 3: Synthesis of sodium (R)-1-methyl-5-(1-tritylaziridine-2-carboxamido)-1H-imidazole-2-carboxylate

To a solution of methyl (R)-1-methyl-5-(1-tritylaziridine-2-carboxamido)-1-imidazole-2-carboxylate (300.0 mg, 0.643 mmol) in THF (3 mL) at room temperature was added NaOH·H₂O (38.6 mg, 0.965 mmol). The resulting solution was warmed to room temperature and stirred for 2 h. The solution was concentrated under reduced pressure to afford the desired product (400 mg, crude). LCMS (ESI) m/z: [M+H] calcd for C₂₇H₂₄N₄O₃: 453.19; found 453.2.

### Intermediate A-61. Synthesis of(S)-1-methyl-5-(1-tritylaziridine-2-carboxamido)-1H-imidazole-2-carboxylic acid

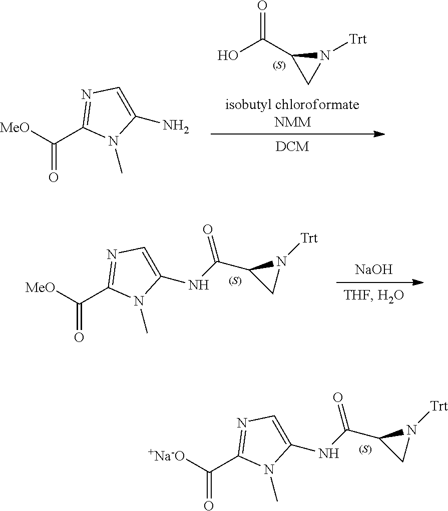

### Step 1: Synthesis of methyl(S)-1-methyl-5-(1-tritylaziridine-2-carboxamido)-1H-imidazole-2-carboxylate

To a solution of(S)-1-tritylaziridine-2-carboxylic acid (1.18 g, 3.577 mmol) in DCM (15 mL) at 0° C. was added isobutyl chloroformate (423.4 mg, 3.100 mmol) and N-methylmorpholine (0.39 mL) 3.862 mmol). The resulting mixture was stirred for 1 h at 0° C. and then methyl 5-amino-1-methyl-1H-imidazole-2-carboxylate (370.0 mg, 2.385 mmol) was added and the resulting mixture was warmed to at room temperature and stirred overnight. The reaction mixture was quenched with sat. aq. NaHCO₃ at 0° C. before being extracted with DCM (2×100 mL). The combined organic layers were washed with brine (150 mL), dried over Na₂SO₄, filtered, and concentrated under reduced pressure. The resulting residue was purified by silica gel chromatography (100% EtOAc) to afford the final product (380 mg, 34.2% yield) as a yellow solid. LCMS (ESI) m/z: [M+Na] calcd for C₂₈H₂₆N₄O₃: 467.21; found 467.3.

### Step 2: Synthesis of(S)-1-methyl-5-(1-tritylaziridine-2-carboxamido)-1H-imidazole-2-carboxylic

To a solution of NaOH (146.6 mg, 3.665 mmol) in H₂O (3.6 mL) at 0° C. was added a solution of methyl(S)-1-methyl-5-(1-tritylaziridine-2-carboxamido)-1H-imidazole-2-carboxylate (380.0 mg, 0.815 mmol) in MeOH (5 mL). The resulting solution was warmed to room temperature and stirred for 6 h. The mixture was acidified to pH 6 with aq. 1 M HCl before being extracted with DCM (2×100 mL). The combined organic layers were washed with brine (150 mL), dried over Na₂SO₄, filtered, and concentrated under reduced pressure to afford the desired product (350 mg, crude). LCMS (ESI) m/z: [M+H] calcd for C₂₇H₂₄N₄O₃: 451.18; found 451.1.

### Intermediate A-62. Synthesis of 4-((2S)-1-(tert-butylsulfinyl) aziridin-2-yl)benzoic acid

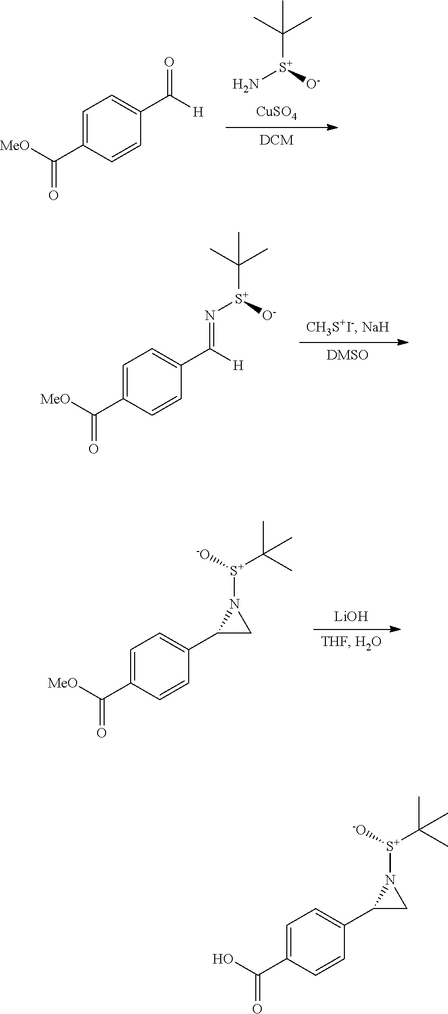

### Step 1: Synthesis of methyl (E)-4-(((tert-butylsulfinyl) imino) methyl)benzoate

To a solution of methyl 4-formylbenzoate (100.0 mg, 0.609 mmol) and 2-methylpropane-2-sulfinamide (76.0 mg, 0.627 mmol) in DCM (2.0 mL) was added CuSO₄ (291.7 mg, 1.827 mmol). The resulting solution was stirred overnight at room temperature and was then filtered. The filter cake was washed with EtOAc (3×200 mL) and the filtrate was concentrated under reduced pressure. The resulting residue was purified by silica gel column chromatography (67% EtOAc/pet. ether) to afford the desired product (2 g, 61.4% yield). LCMS (ESI) m/z: [M+H] calcd for C₁₃H₁₇NO₃S: 268.10; found 268.0.

### Step 2: Synthesis of methyl 4-((2S)-1-(tert-butylsulfinyl) aziridin-2-yl)benzoate

Methyl (E)-4-(((tert-butylsulfinyl) imino) methyl)benzoate (500.0 mg, 1.863 mmol), Me₃S⁺I⁻(1.14 g, 5.590 mmol), and 60% NaH (134.15 mg, 5.590 mmol) were dissolved in DMSO (10.0 mL) at room temperature. The resulting mixture was stirred for 2 h and then the reaction was quenched by the addition of sat. aq. NH₄Cl (10 mL). The resulting mixture was extracted with EtOAc (3×50 mL) and the combined organic layers were washed with brine (2×100 mL), dried over Na₂SO₄, filtered, and concentrated under reduced pressure. The resulting residue was purified by silica gel chromatography (10% EtOAc/pet. ether) to afford the desired product (300 mg, 57.0% yield). LCMS (ESI) m/z: [M+H] calcd for C₁₄H₁₉NO₃S: 282.12; found 282.1.

### Step 3: Synthesis of 4-((2S)-1-(tert-butylsulfinyl) aziridin-2-yl)benzoic acid

To a solution of methyl 4-((2S)-1-(tert-butylsulfinyl) aziridin-2-yl)benzoate (400.0 mg, 1.422 mmol) in THF (5.0 mL) and H₂O (1.0 mL) was added LiOH (103.0 mg, 4.301 mmol). The resulting mixture was stirred overnight at room temperature and was then acidified to PH ˜3 with 1 M HCl. The mixture was extracted with EtOAc (3×200 mL), dried over Na₂SO₄, filtered, and concentrated under reduced pressure. The resulting residue was purified by prep-TLC(10% MeOH/DCM) to afford the desired product (130 mg, 91.2% yield). LCMS (ESI) m/z: [M−H] calcd for C₁₃H₁₇NO₃S: 266.09; found 266.0.

### Intermediate A-63. Synthesis of(S)-3-methyl-2-((R)-2-oxo-3-((R)-1-tritylaziridine-2-carboxamido) pyrrolidin-1-yl) butanoic acid

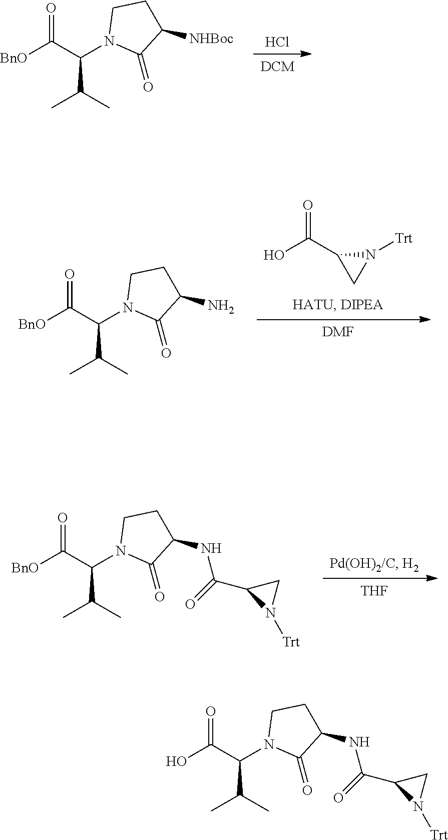

### Step 1: Synthesis of benzyl(S)-2-((R)-3-amino-2-oxopyrrolidin-1-yl)-3-methylbutanoate

To a solution of benzyl(S)-2-((R)-3-((tert-butoxycarbonyl) amino)-2-oxopyrrolidin-1-yl)-3-methylbutanoate (1.0 g, 2.561 mmol) in DCM (10.0 mL) was added 4M HCl in 1,4-dioxane (5.0 mL) at 0° C. The resulting mixture was stirred for 2 h at room temperature under an argon atmosphere. The resulting mixture was concentrated under reduced pressure to afford the desired crude product (890 mg, crude) as a yellow solid. LCMS (ESI) m/z: [M+H] calcd for C₁₆H₂₂N₂O₃: 291.17; found 291.1.

### Step 2: Synthesis of benzyl(S)-3-methyl-2-((R)-2-oxo-3-((R)-1-tritylaziridine-2-carboxamido) pyrrolidin-1-yl) butanoate

To a solution of benzyl(S)-2-((R)-3-amino-2-oxopyrrolidin-1-yl)-3-methylbutanoate (450.0 mg, 1.550 mmol) and (R)-1-tritylaziridine-2-carboxylic acid (765.8 mg, 2.325 mmol) in DMF were added HATU (1.179 g, 3.100 mmol) and DIPEA (1.35 mL, 7.75 mmol) dropwise at 0° C. The resulting mixture was stirred for 2 h at room temperature and was then extracted with EtOAc (2×100 mL). The combined organic layers were washed with H₂O, brine (20 mL), dried over Na₂SO₄, filtered, and concentrated under reduced pressure. The resulting residue was purified by silica gel chromatography (50% EtOAc/pet. ether) to afford the desired product (470 mg, 50.4% yield). LCMS (ESI) m/z: [M+H] calcd for C₃₈H₃₉N₃O₄: 602.31; found 602.3.

### Step 3: Synthesis of(S)-3-methyl-2-((R)-2-oxo-3-((R)-1-tritylaziridine-2-carboxamido) pyrrolidin-1-yl) butanoic acid

A suspension of benzyl(S)-3-methyl-2-((R)-2-oxo-3-((R)-1-tritylaziridine-2-carboxamido) pyrrolidin-1-yl) butanoate (430.0 mg, 0.715 mmol) and Pd (OH) 2/C(230.0 mg, 1.638 mmol) were in THF (5 mL) was stirred for 3 h under and atmosphere of hydrogen (1 atm). The resulting mixture was filtered and the filter cake was washed with MeOH (2×50 mL). The filtrate was concentrated under reduced pressure to afford the crude final product (16 mg, crude) as a white solid. LCMS (ESI) m/z: [M+H] calcd for C₃₁H₃₃N₃O₄: 510.24; found 510.1.

### Intermediate A-64. Synthesis of potassium(S)-1-isopropylaziridine-2-carboxylate

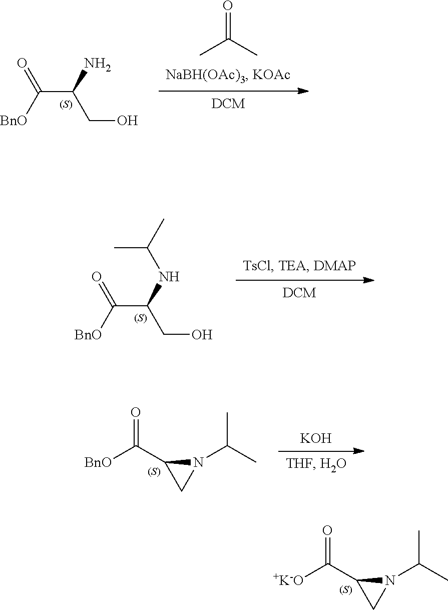

### Step 1: Synthesis benzyl isopropyl-L-serinate

To a solution of benzyl L-serinate (3.65 g, 18.69 mmol), KOAc (1.83 g, 18.69 mmol), and acetone (2.5 mL, 33.66 mmol) in DCM (60.0 mL) was added NaBH(AcO)₃ (4.76 g, 22.436 mmol) in portions at 0° C. The resulting mixture was stirred overnight at room temperature. The reaction was quenched by the addition of sat. aq. NaHCO₃ (50 mL) at room temperature. The resulting mixture was extracted with DCM (3×80 mL). The combined organic layers were washed with brine (50 mL), dried over Na₂SO₄, filtered, and concentrated under reduced pressure. The residue was purified by silica gel chromatography (67% EtOAc/hexanes) to afford the desired product (2.7 g, 60.9% yield) as an off-white solid. LCMS (ESI) m/z: [M+H] calcd for C₁₃H₁₉NO_{3: 238.14}; found 238.2.

### Step 2: Synthesis of benzyl(S)-1-isopropylaziridine-2-carboxylate

To a solution of benzyl isopropyl-L-serinate (2.70 g, 11.378 mmol), Et₃N (4.75 mL, 34.134 mmol) and DMAP (2.57 mg, 0.021 mmol) in DCM (50.0 mL) was added a solution of TsCl (2.60 g, 13.65 mmol) in DCM dropwise at 0° C. The resulting mixture was stirred overnight at room temperature and was then stirred for 4 h at 40° C. The reaction mixture was diluted with H₂O (80 mL) and was then extracted with DCM (2×50 mL). The combined organic layers were washed with brine (30 mL), dried over Na₂SO₄, filtered, and concentrated under reduced pressure. The residue was purified by silica gel chromatography (20% EtOAc/hexanes) to afford the desired product (2.3 g, 93.2% yield). LCMS (ESI) m/z: [M+H] calcd for C₁₃H₁₇NO₂: 220.13; found 220.1.

### Step 3: Synthesis of potassium(S)-1-isopropylaziridine-2-carboxylate

To a solution of benzyl(S)-1-isopropylaziridine-2-carboxylate (800.0 mg, 3.65 mmol) and H₂O (6.0 mL) and THF (8.0 mL) was added a solution of KOH (245.62 mg, 4.378 mmol) in H₂O (2.0 mL) dropwise at 0° C. The resulting mixture was stirred for 2 h at room temperature. The mixture was diluted with H₂O (10 mL) and the aqueous layer was washed with MTBE (3×8 mL). The aqueous layer was dried by lyophilization to afford the desired product (400 mg, crude). LCMS (ESI) m/z: [M+H] calcd for C₆H₁₁NO₂: 130.09; found 130.0.

### Intermediate A-65. Synthesis of potassium (R)-1-isopropylaziridine-2-carboxylate

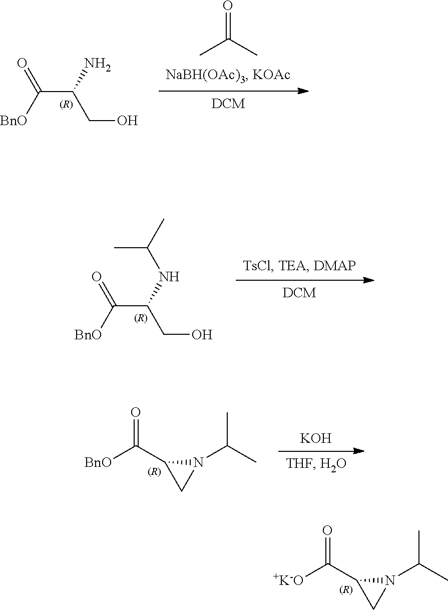

### Step 1: Synthesis benzyl isopropyl-D-serinate

To a solution of benzyl D-serinate (2.10 g, 10.757 mmol), KOAc (1.06 g, 10.757 mmol), and acetone (1.2 mL, 16.136 mmol) in DCM (40.0 mL) was added a solution of NaBH(AcO)₃ (2.96 g, 13.984 mmol) in portions at 0° C. The resulting mixture was stirred overnight at room temperature. The reaction was quenched by the addition of sat. aq. NaHCO₃ (50 mL) and the mixture was extracted with DCM (3×50 mL). The combined organic layers were washed with brine (50 mL), dried over Na₂SO₄, filtered, and concentrated under reduced pressure. The residue was purified by silica gel chromatography (67% EtOAc/hexanes) to afford the desired product (1.7 g, 66.6% yield). LCMS (ESI) m/z: [M+H] calcd for C₁₃H₁₉NO₃: 238.14; found 238.0.

### Step 2: Synthesis of benzyl (R)-1-isopropylaziridine-2-carboxylate

To a solution of benzyl isopropyl-D-serinate (1.75 g, 7.375 mmol), Et₃N (2.58 mL, 18.437 mmol) and DMAP (90.09 mg, 0.737 mmol) in DCM (30.0 mL) was added a solution of TsCl (1.69 g, 8.850 mmol) in DCM dropwise at 0° C. The resulting mixture was stirred overnight at room temperature before being stirred for 4 h at 40° C. The mixture was diluted with H₂O (80 mL) and then extracted with DCM (3×50 mL). The combined organic layers were washed with brine (50 mL), dried over Na₂SO₄, filtered, and concentrated under reduced pressure. The residue was purified by silica gel chromatography (20% EtOAc/hexanes) to afford the desired product (1.4 g, 86.6% yield). LCMS (ESI) m/z: [M+H] calcd for C₁₃H₁₇NO₂: 220.13; found 219.9.

### Step 3: Synthesis of potassium (R)-1-isopropylaziridine-2-carboxylate

To a solution of benzyl (R)-1-isopropylaziridine-2-carboxylate (600.0 mg, 2.736 mmol) in H₂O (3.0 mL) and THF (5.0 mL) was added a solution of KOH (184.22 mg, 3.283 mmol) in H₂O (2.0 mL) dropwise at 0° C. The resulting mixture was stirred for 2 h at room temperature. The mixture was then diluted with H₂O (10 mL) and the aqueous layer was washed with MTBE (3×8 mL). The aqueous layer was then dried by lyophilization to afford the desired product (260 mg, crude). LCMS (ESI) m/z: [M+H] calcd for C₆H₁₁NO₂: 130.09; found 130.1.

### Intermediate A-66. Synthesis of N-methyl-N-(3-oxo-4-((R)-1-tritylaziridine-2-carbonyl) piperazine-1-carbonyl)-L-valine

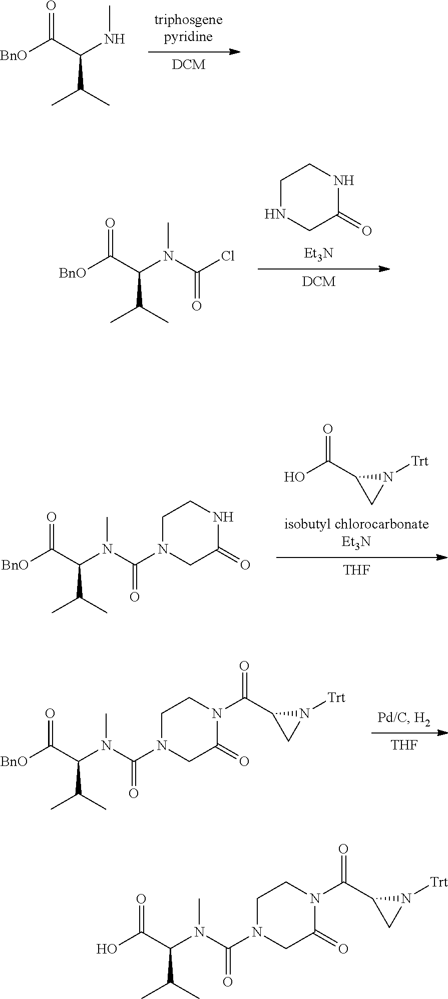

### Step 1: Synthesis of benzyl N-(chlorocarbonyl)-N-methyl-L-valinate

To a solution of benzyl methyl-L-valinate (2.0 g, 9.038 mmol) in DCM (20.0 mL) was added a solution of triphosgene (800 mg, 2.711 mmol) and pyridine (2.14 g, 27.113 mmol) in DCM (20.0 mL) dropwise at 0° C. The resulting mixture was stirred for 2 h at room temperature before being diluted with EtOAc. The solution was stirred for 30 min at room temperature and was then filtered. The filtrate was concentrated under reduced pressure to afford the crude product which was used in the next step directly without further purification. LCMS (ESI) m/z: [M+H] calcd for C₁₄H₁₈ClNO₃: 284.11; found 283.1.

### Step 2: Synthesis of benzyl N-methyl-N-(3-oxopiperazine-1-carbonyl)-L-valinate

To a solution of piperazin-2-one (100.0 mg, 0.999 mmol) and Et₃N (0.487 mL, 3.496 mmol) in DCM (5.0 mL) was added a solution of benzyl N-(chlorocarbonyl)-N-methyl-L-valinate (311.75 mg, 1.099 mmol) in DCM (5 mL) dropwise at 0° C. The resulting mixture was stirred for 4 h at room temperature. The mixture was then diluted with H₂O (5 mL), the aqueous layer was extracted with DCM (3×5 mL), and the combined organic layers were concentrated under reduced pressure. The residue was purified by prep-TLC(100% EtOAc) to afford the desired product (200 mg, 57.6% yield). LCMS (ESI) m/z: [M+H] calcd for C₁₈H₂₅N₃O₄: 348.19; found 348.1.

### Step 3: Synthesis of benzyl N-methyl-N-(3-oxo-4-((R)-1-tritylaziridine-2-carbonyl) piperazine-1-carbonyl)-L-valinate

To a solution of (R)-1-tritylaziridine-2-carboxylic acid (711.11 mg, 2.159 mmol) in THF was added Et₃N (0.40 mL, 2.878 mmol) and isobutyl chlorocarbonate (255.53 mg, 1.871 mmol) dropwise at 0° C. under a nitrogen atmosphere. The resulting mixture was stirred for 1 h at room temperature and then benzyl N-methyl-N-(3-oxopiperazine-1-carbonyl)-L-valinate (500.0 mg, 1.439 mmol) was added at room temperature. The resulting mixture was warmed to 70° C. and stirred overnight. The reaction was then cooled to room temperature and concentrated under reduced pressure. The residue was purified by prep-TLC(EtOAc/50% pet. ether) to afford the desired product (200 mg, 21.1% yield). LCMS (ESI) m/z: [M+H] calcd for C₄₀H₄₂N₄O₅: 659.32; found 677.4.

### Step 4: Synthesis of N-methyl-N-(3-oxo-4-((R)-1-tritylaziridine-2-carbonyl) piperazine-1-carbonyl)-L-valine

A suspension of benzyl N-methyl-N-(3-oxo-4-((R)-1-tritylaziridine-2-carbonyl) piperazine-1-carbonyl)-L-valinate (140.0 mg, 0.212 mmol) and Pd/C (50.0 mg) in THF (3 mL) was stirred for 2 h under a hydrogen atmosphere (1 atm). The mixture was then filtered and the filter cake was washed with MeOH (3×15 mL). The filtrate was concentrated under reduced pressure to afford the desired product (100 mg, crude). LCMS (ESI) m/z: [M−H] calcd for C₃₃H₃₆N₄O₅: 567.26; found 567.1.

### Intermediate A-67. Synthesis of(S)-1-(2-((tert-butyldiphenylsilyl)oxy) ethyl) aziridine-2-carboxylic acid

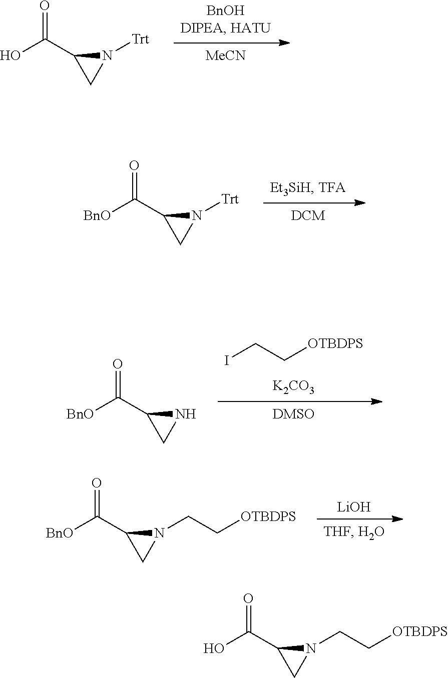

### Step 1: Synthesis of benzyl(S)-1-tritylaziridine-2-carboxylate

To a solution of(S)-1-tritylaziridine-2-carboxylic acid (500.0 mg, 1.518 mmol), benzyl alcohol (246.2 mg, 2.277 mmol) and DIPEA (0.793 mL, 4.554 mmol) in MeCN (10.0 mL) was added HATU (1.73 mg, 4.554 mmol). The resulting solution was stirred for 3 h at room temperature and was then concentrated under reduced pressure. The crude residue was purified by prep-TLC(50% EtOAc/pet. ether) to afford the desired product (300 mg, 47.1% yield) as an off-white slid. LCMS (ESI) m/z: [M+Na] calcd for C₂₉H₂₅NO₂: 442.18; found 442.3.

### Step 2: Synthesis of benzyl(S)-aziridine-2-carboxylate

To a solution of benzyl(S)-1-tritylaziridine-2-carboxylate (300.0 mg, 0.715 mmol) in DCM (5.0 mL) at 0° C. was added TFA (326.2 mg, 2.860 mmol) and Et₃SiH (332.6 mg, 2.860 mmol). The resulting mixture was stirred at 0° C. for 3 h and was then concentrated under reduced pressure. The residue was purified by prep-TLC(10% MeOH/DCM) to afford the desired product (130 mg, 82.1% yield). LCMS (ESI) m/z: [M+H] calcd for C₁₀H₁₁NO₂: 178.09; found 178.2.

### Step 3: Synthesis of benzyl(S)-1-(2-((tert-butyldiphenylsilyl)oxy) ethyl) aziridine-2-carboxylate

To a solution of benzyl(S)-aziridine-2-carboxylate (400.0 mg, 2.257 mmol) and tert-butyl (2-iodoethoxy) diphenylsilane (1.85 g, 4.52 mmol) in DMSO (10.0 mL) was added K₂CO₃ (935.9 mg, 6.772 mmol) at room temperature. The mixture was stirred at 60° C. for 5 h. The mixture was diluted with H₂O (30.0 mL) and was extracted with EtOAc (2×30 mL). The combined organic layers were washed with brine (2×50 mL), dried over Na₂SO₄, filtered, and concentrated under reduced pressure. The resulting residue was purified by prep-TLC(20% EtOAc/pet. ether) to afford the desired product (200 mg, 15.4% yield). LCMS (ESI) m/z: [M+H] calcd for C₂₈H₃₃NO₃Si: 460.23; found 460.0.

### Step 4: Synthesis of lithium(S)-1-(2-((tert-butyldiphenylsilyl)oxy) ethyl) aziridine-2-carboxylate

To a solution of benzyl(S)-1-(2-((tert-butyldiphenylsilyl)oxy) ethyl) aziridine-2-carboxylate (200.0 mg, 0.435 mmol) in MeOH (2.0 mL) was added LiOH·H₂O (36.5 mg, 0.870 mmol). The resulting mixture was stirred overnight and was then concentrated under reduced pressure to afford the desired product (200 mg, crude). LCMS (ESI) m/z: [M+H] calcd for C₂₁H₂₇NO₃Si: 370.18; found 370.1.

### Intermediate A-68. Synthesis of (R)-1-(2-((tert-butyldiphenylsilyl)oxy) ethyl) aziridine-2-carboxylic acid

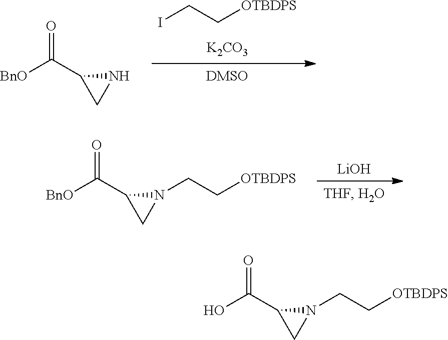

### Step 1: Synthesis of methyl benzyl (R)-1-(2-((tert-butyldiphenylsilyl)oxy) ethyl) aziridine-2-carboxylate

To a solution of benzyl (R)-aziridine-2-carboxylate (600.0 mg, 3.386 mmol) and K₂CO₃ (1.87 g, 13.544 mmol) in DMSO (8.0 mL) was added tert-butyl (2-iodoethoxy) diphenylsilane (1.39 g, 3.386 mmol) in portions at room temperature. The resulting mixture was stirred at 80° C. for 16 h. The reaction mixture was then cooled to room temperature and concentrated under reduced pressure. The residue was purified by reverse phase chromatography (60→90% MeCN/H₂O) to afford the desired product (150 mg, 9.6% yield) as a colorless solid. LCMS (ESI) m/z: [M+Na] calcd for C₂₈H₃₃NO₃Si: 482.21; found 482.3.

### Step 2: Synthesis of (R)-1-(2-((tert-butyldiphenylsilyl)oxy) ethyl) aziridine-2-carboxylic acid

To a solution of methyl benzyl (R)-1-(2-((tert-butyldiphenylsilyl)oxy) ethyl) aziridine-2-carboxylate (180.0 mg, 0.392 mmol) in H₂O (2.0 mL) and THF (3.0 mL) at 0° C. was added a solution of LiOH·H₂O (32.87 mg, 0.392 mmol) in H₂O (1.0 mL). The resulting mixture was diluted with H₂O (6.0 mL) and the aqueous layer was washed with MTBE (3×4 mL). The aqueous layer was dried by lyophilization which afforded the desired product (140 mg, crude). LCMS (ESI) m/z: [M+H] calcd for C₂₁H₂₇NO₃Si: 370.18; found 370.0.

### Intermediate A-69. Synthesis of 6-((2S)-1-(tert-butylsulfinyl) aziridin-2-yl) nicotinic acid

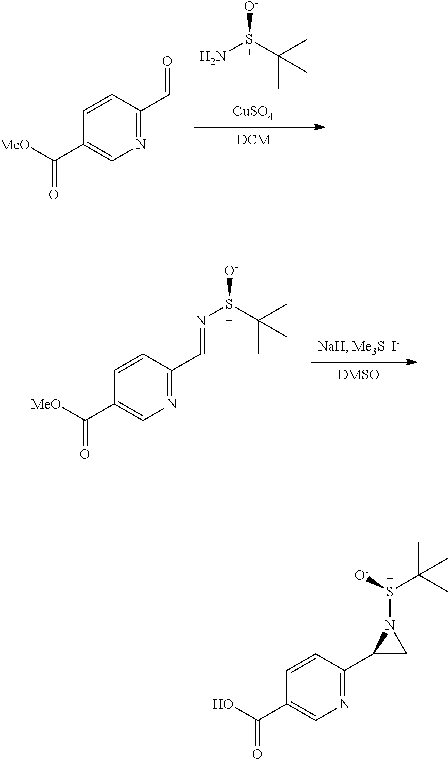

### Step 1: Synthesis of methyl (E)-6-(((tert-butylsulfinyl) imino) methyl) nicotinate

To a mixture of methyl 6-formylnicotinate (2.0 g, 12.11 mmol) and 2-methylpropane-2-sulfinamide (2.94 g, 24.26 mmol) in DCM (60 mL) was added CuSO₄ (5.80 g, 36.34 mmol). The resulting mixture was stirred at room temperature for 18 h. The reaction mixture was filtered, the filter cake was washed with DCM (3×30 mL), and the filtrate was concentrated under reduced pressure. Purification by normal phase chromatography (66% EtOAc/pet. ether) afforded the desired product (2.581 g, 80% yield). LCMS (ESI) m/z: [M+H] calcd for C₁₂H₁₆N₂O₃S: 269.10; found 269.1.

### Step 2: Synthesis of 6-((2S)-1-(tert-butylsulfinyl) aziridin-2-yl) nicotinic acid

To a suspension of NaH (60%, 179.76 mg, 7.491 mmol) in DMSO (20 mL) at 0° C. was added MesS⁺I⁻ (1.53 g, 7.491 mmol) and the resulting mixture was warmed to room temperature and stirred for 1 h. To the reaction mixture was added a solution of methyl (E)-6-(((tert-butylsulfinyl) imino) methyl) nicotinate (670.0 mg, 2.497 mmol) in DMSO (20 mL) in portions. The mixture was stirred at room temperature for 3 h and was then diluted with EtOAc. The mixture was acidified to pH 4 with 1 M HCl and then the aqueous layer was extracted with EtOAc. The combined organic layers were concentrated under reduced pressure. Purification by reverse phase chromatography (10→15% MeCN/H₂O) afforded the desired product (313 mg, 45% yield). LCMS (ESI) m/z: [M+H] calcd for C₁₂H₁₆N₂O₃S: 269.10; found 269.1.

### Intermediate A-70. Synthesis of N-methyl-N—(N-methyl-N—((R)-1-tritylaziridine-2-carbonyl)-D-alanyl)-L-valine

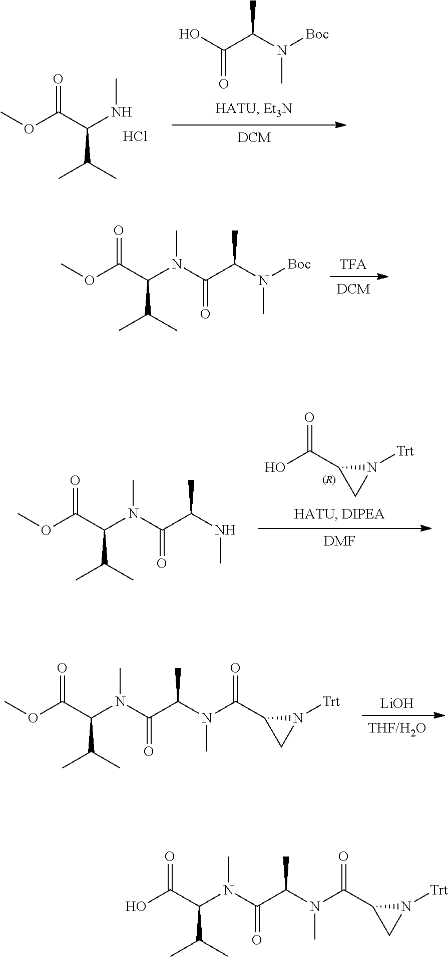

### Step 1: Synthesis of methyl N—(N-(tert-butoxycarbonyl)-N-methyl-D-alanyl)-N-methyl-L-valinate

To a solution of methyl-L-valinate hydrochloride (1.0 g, 5.51 mmol) and N-(tert-butoxycarbonyl)-N-methyl-D-alanine (1.34 g, 6.59 mmol) in DCM (20.0 mL) at 0° C. was added Et₃N (2.3 mL, 16.51 mmol) and HATU (2.72 g, 7.16 mmol). The mixture was warmed to room temperature and stirred for 4 h. The reaction mixture was then diluted with DCM (20 mL) and washed with sat. aq. NH₄Cl (2×40 mL) and brine (40 mL), dried over Na₂SO₄, filtered, and concentrated under reduced pressure. The residue was purified by silica gel column chromatography (20% EtOAc/pet. ether) to afford the desired product (1.5 g, 82.5% yield). LCMS (ESI) m/z: [M+H] calcd for C₁₆H₃₀N₂O₅: 331.23; found 331.1.

### Step 2: Synthesis of methyl N-methyl-N-(methyl-D-alanyl)-L-valinate

To a solution of methyl N—(N-(tert-butoxycarbonyl)-N-methyl-D-alanyl)-N-methyl-L-valinate (1.50 g, 4.54 mmol) in DCM (9.0 mL) at 0° C. was added TFA (4.5 mL). The resulting mixture was warmed to room temperature and stirred for 1 h. The reaction mixture was concentrated under reduced pressure to afford the desired product (1 g, crude). LCMS (ESI) m/z: [M+H] calcd for C₁₁H₂₂N₂O₃: 231.17; found 231.2.

### Step 3: Synthesis of methyl N-methyl-N—(N-methyl-N—((R)-1-tritylaziridine-2-carbonyl)-D-alanyl)-L-valinate

To a solution of methyl N-methyl-N-(methyl-D-alanyl)-L-valinate (900.0 mg, 3.91 mmol) and (R)-1-tritylaziridine-2-carboxylic acid (1.544 g, 4.689 mmol) in DMF (20.0 mL) at 0° C. was added DIPEA (3.4 mL, 19.54 mmol) and HATU (2.228 g, 5.86 mmol). The mixture was warmed to room temperature and stirred for 1 h. The reaction mixture was diluted with H₂O (50 mL) and extracted with EtOAc (3×50 mL). The combined organic layers were dried over Na₂SO₄, filtered, and concentrated under reduced pressure. The residue was purified by silica gel column chromatography (33% EtOAc/pet. ether) to afford the desired product (1.2 g, 56.7% yield). LCMS (ESI) m/z: [M+H] calcd for C₃₃H₃₉N₃O₄: 542.30; found 542.3.

### Step 4: Synthesis of N-methyl-N—(N-methyl-N—((R)-1-tritylaziridine-2-carbonyl)-D-alanyl)-L-valine

To a solution of methyl N-methyl-N—(N-methyl-N—((R)-1-tritylaziridine-2-carbonyl)-D-alanyl)-L-valinate (200.0 mg, 0.369 mmol) in THF (2.0 mL) at 0° C. was added a solution of LiOH·H₂O (77 mg, 1.84 mmol) in H₂O (1.85 mL). The resulting mixture was warmed to room temperature and stirred overnight. The mixture was adjusted to pH 9 with 1 M HCl and then adjusted to pH 7 with aq. NH₄Cl. The aqueous layer was extracted with EtOAc (3×20 mL). The combined organic layers were dried over Na₂SO₄, filtered, and concentrated under reduced pressure to afford the desired product (200 mg, crude. LCMS (ESI) m/z: [M+H] calcd for C₃₂H₃₇N₃O₄: 528.29; found 528.3.

### Intermediate A-71 and A-72. Synthesis of (2R,3S)-1-(4-methoxybenzyl)-3-(trifluoromethyl) aziridine-2-carboxylic acid and (2S,3R)-1-(4-methoxybenzyl)-3-(trifluoromethyl) aziridine-2-carboxylic acid

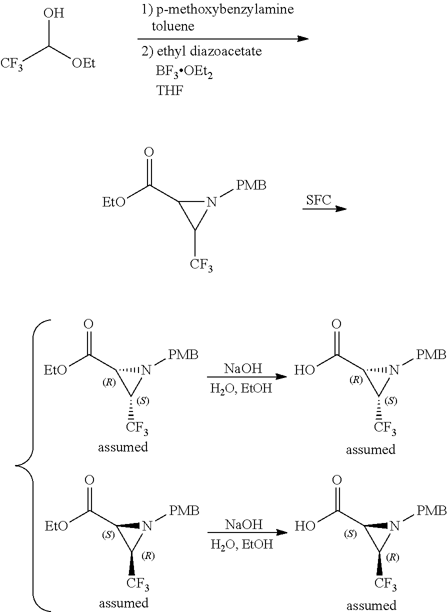

### Step 1: Synthesis of ethyl 1-(4-methoxybenzyl)-3-(trifluoromethyl) aziridine-2-carboxylate

A solution of 1-ethoxy-2,2,2-trifluoroethan-1-ol (2.17 mL, 18.37 mmol) and p-methoxybenzylamine (1.89 mL, 14.58 mmol) in toluene (46 mL) was refluxed for 16 h under Dean-Stark conditions. The reaction was concentrated under reduced pressure and the resulting residue was dissolved in THF (80 mL) and cooled to −78° C. BF₃·Et₂O (0.360 mL, 2.92 mmol) was added to the solution, followed by dropwise addition of ethyl diazoacetate (1.83 mL, 17.50 mmol). The reaction was stirred for 4 h at room temperature. The reaction mixture was quenched by addition of aq. sat. NaHCO₃ (5 mL), and the resulting solution was extracted with DCM (3×50 mL). The combined organic layers were washed with H₂O (20 mL) and brine (10 mL). The organic phase was dried over Na₂SO₄, filtered, and concentrated under reduced pressure. The residue was purified by silica gel column chromatography (1→10% EtOAc/pet. ether) afford the desired product (2 g, 45.2 yield).

### Step 2: Synthesis of ethyl (2R,3S)-1-(4-methoxybenzyl)-3-(trifluoromethyl) aziridine-2-carboxylate and ethyl (2S,3R)-1-(4-methoxybenzyl)-3-(trifluoromethyl) aziridine-2-carboxylate

Ethyl 1-(4-methoxybenzyl)-3-(trifluoromethyl) aziridine-2-carboxylate (1 g) was purified by SFC separation (column: REGIS (S,S) WHELK-01 (250 mm*25 mm, 10 μm); mobile phase: [Neu-IPA]; B %: 13%-13%, min) to afford ethyl (2R,3S)-1-(4-methoxybenzyl)-3-(trifluoromethyl) aziridine-2-carboxylate (530 mg) and ethyl (2S,3R)-1-(4-methoxybenzyl)-3-(trifluoromethyl) aziridine-2-carboxylate (470 mg).

### Step 3: Synthesis of (2R,3S)-1-(4-methoxybenzyl)-3-(trifluoromethyl) aziridine-2-carboxylic acid

To a solution of ethyl (2R,3S)-1-(4-methoxybenzyl)-3-(trifluoromethyl) aziridine-2-carboxylate (430 mg, 1.42 mmol) in EtOH (4 mL) and H₂O (6 mL) was added NaOH (113.42 mg, 2.84 mmol). The mixture was stirred at room temperature for 5 h. The mixture was acidified with aq. HCl (2M) to pH=1-2. The reaction mixture was poured into H₂O (3 mL) and the aqueous phase was extracted with EtOAc (3×3 mL). The combined organic phase was washed with brine (5 ml), dried over Na₂SO₄, filtered, and concentrated under reduced pressure to afford the desired product (350 mg, 89.1% yield). LCMS (ESI) m/z: [M+H] calcd for C₁₂H₁₁FNO_{3: 274.08}; found 274.1 .

### Step 4: Synthesis of (2S,3R)-1-(4-methoxybenzyl)-3-(trifluoromethyl) aziridine-2-carboxylic acid

To a solution of ethyl (2S,3R)-1-(4-methoxybenzyl)-3-(trifluoromethyl) aziridine-2-carboxylate (370 mg, 1.22 mmol) in H₂O (2 mL) and EtOH (4 mL) was added NaOH (97.59 mg, 2.44 mmol). The mixture was stirred at room temperature for 5 h. The mixture was brought to pH=1-2 with the addition of aq. HCl (2 M). The reaction mixture was poured into H₂O (3 mL) and the aqueous phase was extracted with EtOAc (3×3 mL). The combined organic phase was washed with brine (5 mL), dried over Na₂SO₄, filtered, and concentrated under reduced pressure to afford the desired product (300 mg, 89.0% yield). LCMS (ESI) m/z: [M+H] calcd for C₁₂H₁₁FNO₃: 234.08; found 234.2 .

### Intermediate A-73 and A-74. Synthesis of (2S,3S)-1-benzyl-3-(trifluoromethyl) aziridine-2-carboxylic acid and (2R,3R)-1-benzyl-3-(trifluoromethyl) aziridine-2-carboxylic acid

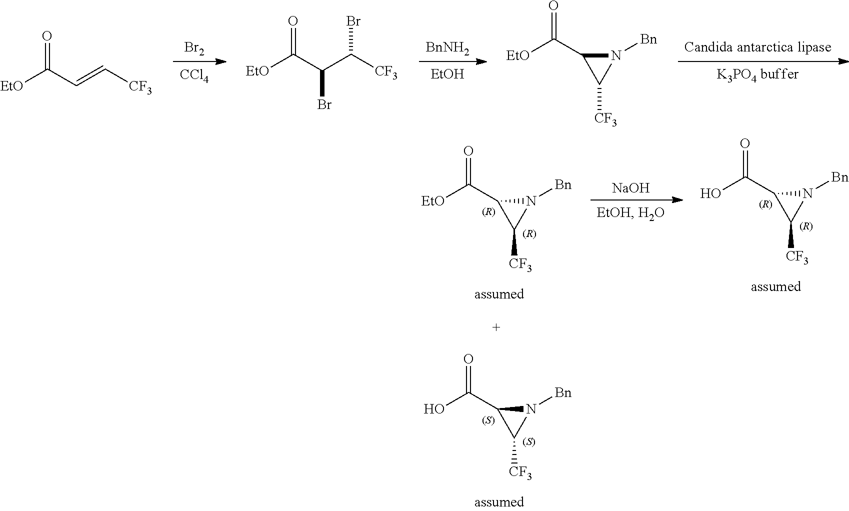

### Step 1: Synthesis of ethyl (2S,3R)-2,3-dibromo-4,4,4-trifluorobutanoate

To a solution of ethyl (E)-4,4,4-trifluorobut-2-enoate (5 g, 29.74 mmol, 4.42 mL) in CCl₄ (90 mL) was added Br₂ (1.69 mL, 32.72 mmol) and the solution was stirred at 75° C. for 5 h. The reaction mixture was concentrated under reduced pressure to give the desired product (10.72 g, crude).

### Step 2: Synthesis of ethyl (2S,3S)-1-benzyl-3-(trifluoromethyl) aziridine-2-carboxylate

To a solution of ethyl (2S,3R)-2,3-dibromo-4,4,4-trifluorobutanoate (10.72 g, 32.69 mmol) in EtOH (30 mL) was slowly added the solution of BnNH₂ (12.47 mL, 114.42 mmol) in EtOH (120 mL) at −5° C. under N₂. The mixture was warmed to room temperature and stirred for 15 h. The mixture was concentrated under reduced pressure and EtOAc (120 mL) was added to the residue. The precipitate was filtered off and the filtrate was washed with aqueous HCl (3%, 180 mL) and H₂O (100 mL), dried over Na₂SO₄, filtered and concentrated under reduced pressure. The residue was purified by silica gel chromatography (20% EtOAc/pet. ether) to afford the desired product (6.02 g, 67.4% yield).

### Step 3: Synthesis of ethyl (2R,3R)-1-benzyl-3-(trifluoromethyl) aziridine-2-carboxylate and (2S,3S)-1-benzyl-3-(trifluoromethyl) aziridine-2-carboxylic acid

Ethyl (2R,3R)-1-benzyl-3-(trifluoromethyl) aziridine-2-carboxylate and (2S,3S)-1-benzyl-3-(trifluoromethyl) aziridine-2-carboxylic acid were synthesized in Enzyme Screening Platform, based on the procedure in Tetrahedron Asymmetry 1999, 10, 2361.

### Step 5: Synthesis of (2R,3R)-1-benzyl-3-(trifluoromethyl) aziridine-2-carboxylic acid

To a solution of ethyl (2R,3R)-1-benzyl-3-(trifluoromethyl) aziridine-2-carboxylate (200 mg, 731.93 μmol) in EtOH (5 mL) was added NaOH (2 M, 548.95 μL) and the mixture was stirred at room temperature for 1 h. The reaction mixture was concentrated under reduced pressure to remove EtOH. Then to the mixture was added HCl (1 M) to adjust pH to 1, and extracted with EtOAc (3×5 mL). The combined organic layers were washed with brine (2×10 mL), dried over Na₂SO₄, filtered and concentrated under reduced pressure to afford the desired product (138 mg, 76.9% yield). LCMS (ESI) m/z: [M+H] calcd for C₁₁H₁₀F₃NO₂: 246.07; found 245.9.

### Intermediate A-75. Synthesis of 1-(oxetan-3-yl) aziridine-2-carboxylic acid

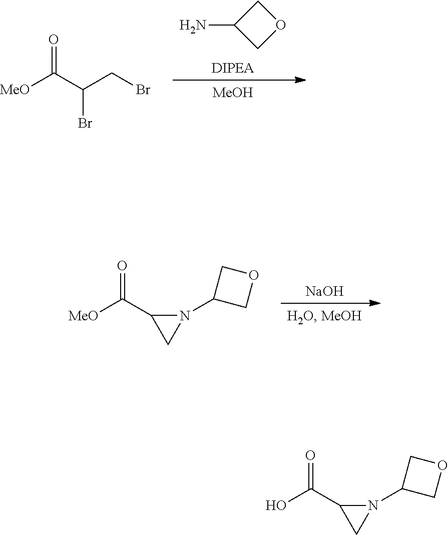

### Step 1: Synthesis of methyl 1-(oxetan-3-yl) aziridine-2-carboxylate

To a solution of methyl 2,3-dibromopropanoate (515.46 UL, 4.07 mmol) in MeOH (15 mL) was added DIPEA (3.54 mL, 20.33 mmol). After addition, the mixture was stirred for 15 min, and then oxetan-3-amine (297.25 mg, 4.07 mmol) was added dropwise. The resulting mixture was stirred at room temperature for 12 h. The reaction mixture was poured into H₂O (20 mL), the aqueous phase was extracted with DCM (2×25 mL). The combined organic phase was washed with brine (20 mL), dried with Na₂SO₄, filtered and concentrated under reduced pressure. The residue was purified by silica gel chromatography (10%->30% EtOAc/pet. ether) to afford the desired product (380 mg, 59.5% yield).

### Step 2: Synthesis of 1-(oxetan-3-yl) aziridine-2-carboxylic acid

To a solution of methyl 1-(oxetan-3-yl) aziridine-2-carboxylate (280 mg, 1.78 mmol) in EtOH (3 mL) was added NaOH (2 M, 1.34 mL) at room temperature and the resulting mixture was stirred for 3 h. The reaction mixture was adjusted to pH 8 by the addition of HCl (1 M), and lyophilized to afford the desired product (200 mg, 78.4% yield).

### Intermediate A-76. Synthesis of (2S,3S)-1-((S)-tert-butylsulfinyl)-3-cyclobutylaziridine-2-carboxylic acid

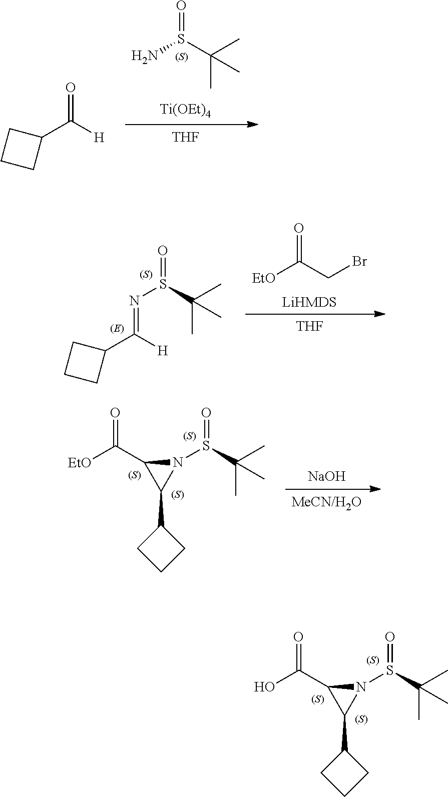

### Step 1: Synthesis of (S,E)-N-(cyclobutylmethylene)-2-methylpropane-2-sulfinamide

To a solution of cyclobutanecarbaldehyde (0.5 g, 5.94 mmol) in THF (10 mL) was added(S) -2-methylpropane-2-sulfinamide (792.48 mg, 6.54 mmol) and Ti(OEt)₄ (2.47 mL, 11.89 mmol).

The mixture was stirred at 75° C. for 3 h. The reaction mixture was cooled to room temperature and quenched by addition brine (30 mL), and filtered to remove solids. The mixture was extracted with EtOAc (3×30 mL). The combined organic layers were washed with brine (2×10 mL), dried over Na₂SO₄, filtered and concentrated under reduced pressure to give a residue. The residue was purified by silica gel chromatography (2%->10% EtOAc/pet. ether) to afford the desired product (907.3 mg, 39.9% yield). LCMS (ESI) m/z: [M+H] calcd for C₉H₁₇NOS: 188.1; found 188.3.

### Step 2: Synthesis of ethyl (2S,3S)-1-((S)-tert-butylsulfinyl)-3-cyclobutylaziridine-2-carboxylate

To a solution of ethyl 2-bromoacetate (1.60 g, 9.61 mmol, 1.06 mL) in THF (9 mL) was added LiHMDS (1 M, 9.61 mL) at −78° C., after 2 min, (S,E)-N-(cyclobutylmethylene)-2-methylpropane-2-sulfinamide (0.9 g, 4.81 mmol) was added. The mixture was stirred at −78° C. for 2 h. The reaction mixture was quenched by addition H₂O (25 mL) at −78° C. and warmed to room temperature, then the mixture extracted with EtOAc (3×20 mL). The combined organic layers were washed with brine (2×5 mL), dried over Na₂SO₄, filtered and concentrated under reduced pressure to give a residue, which was purified by silica gel chromatography (10%->20% EtOAc/pet. ether) to afford the desired product (426 mg, crude). LCMS (ESI) m/z: [M+H] calcd for C₁₃H₂₃NO₃S: 274.14; found 274.3.

### Step 3: Synthesis of (2S,3S)-1-((S)-tert-butylsulfinyl)-3-cyclobutylaziridine-2-carboxylic acid

To a solution of (2,3S)-1-((S)-tert-butylsulfinyl)-3-cyclobutylaziridine-2-carboxylate (100 mg, 365.78 μmol) in MeCN (0.5 mL) and H₂O (0.5 mL) was added NaOH (21.95 mg, 548.67 μmol) at 0° C., the mixture was warmed to room temperature and stirred for 2 h. The reaction mixture was adjusted to pH 5 by addition aq. 10% citric acid (˜10 mL) and was then extracted with EtOAc (3×20 mL). The combined organic layers were washed with brine (2×5 mL), dried over Na₂SO₄, filtered and concentrated under reduced pressure to afford the desired product (92.6 mg, crude). LCMS (ESI) m/z: [M+H] calcd for C₁₁H₁₉NO₃S: 246.11; found 246.3.

### Intermediate A-77. Synthesis of (2R,3R)-1-((R)-tert-butylsulfinyl)-3-cyclobutylaziridine-2-carboxylic acid

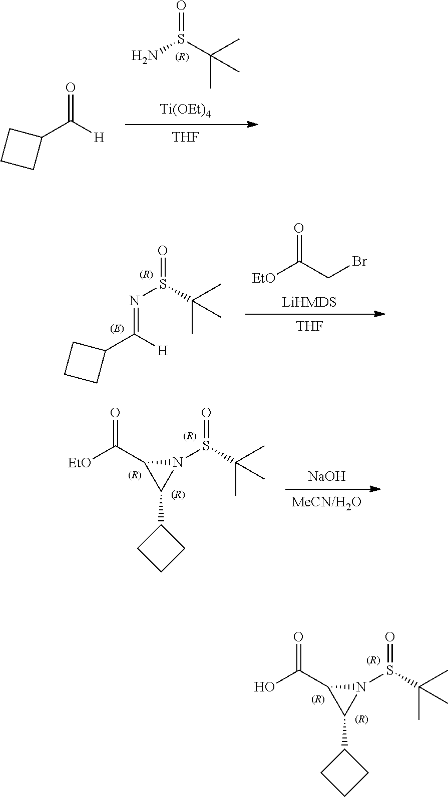

### Step 1: Synthesis of (R,E)-N-(cyclobutylmethylene)-2-methylpropane-2-sulfinamide

To a solution of cyclobutanecarbaldehyde (0.25 g, 2.97 mmol) in THF (5 mL) was added (R)-2-methylpropane-2-sulfinamide (396.24 mg, 3.27 mmol) and Ti(OEt)₄ (1.36 g, 5.94 mmol, 1.23 mL). The mixture was stirred at 75° C. for 3 h in two batches. The two batches were combined and the reaction mixture was quenched by the addition of brine (15 mL). The solution was extracted with EtOAc (3×20 mL) and the combined organic layers were washed with brine (2×5 mL), dried over Na₂SO₄, filtered and concentrated under reduced pressure to give a residue, which was purified by silica gel chromatography (10%->20% EtOAc/pet. ether) to afford the desired product (786.7 mg, 70.7% yield). LCMS (ESI) m/z: [M+H] calcd for C₉H₁₇NOS: 188.1; found 188.3.

### Step 2: Synthesis of ethyl (2R,3R)-1-((R)-tert-butylsulfinyl)-3-cyclobutylaziridine-2-carboxylate

To a solution of ethyl 2-bromoacetate (236.19 μL, 2.14 mmol) in THF (2 mL) was added LiHMDS (1 M, 2.14 mL) at −78° C., after 30 min, (R,E)-N-(cyclobutylmethylene)-2-methylpropane-2-sulfinamide (0.2 g, 1.07 mmol) was added. The mixture was warmed to −40° C. and stirred for 4 h. The reaction mixture was quenched by addition H₂O (18 mL) at −40° C. and warmed to room temperature The mixture was extracted with EtOAc (3×15 mL) and the combined organic layers were washed with brine (2×5 mL), dried over Na₂SO₄, filtered and concentrated under reduced pressure to give a residue, which was purified by prep-TLC(20% EtOAc/pet. ether) to afford the desired product (0.1 g, crude). LCMS (ESI) m/z: [M+H] calcd for C₁₃H₂₃NO₃S: 274.14; found 274.3.

### Step 3: Synthesis of (2R,3R)-1-((R)-tert-butylsulfinyl)-3-cyclobutylaziridine-2-carboxylic acid

In two batches, to a solution ethyl (2R,3R)-1-((R)-tert-butylsulfinyl)-3-cyclobutylaziridine-2-carboxylate (25 mg, 91.44 μmol) in MeCN (0.25 mL) and H₂O (0.25 mL) was added NaOH (5.49 mg, 137.17 μmol) at 0° C., the mixture was warmed to room temperature and stirred for 5 h. The reaction mixtures were combined, and adjust to pH to 5 with aq. 10% citric acid (10 mL), then extracted with EtOAc (3×20 mL). The combined organic layers were washed with brine (2×5 mL), dried over Na₂SO₄, filtered and concentrated under reduced pressure to afford the desired product (53 mg, crude). LCMS (ESI) m/z: [M+H] calcd for C₁₁H₁₉NO₃S: 246.11; found 246.2.

### Intermediate A-78. Synthesis of N-methyl-N-(methyl ((S)-1-((R)-1-tritylaziridine-2-carbonyl) piperidin-3-yl) carbamoyl)-L-valine

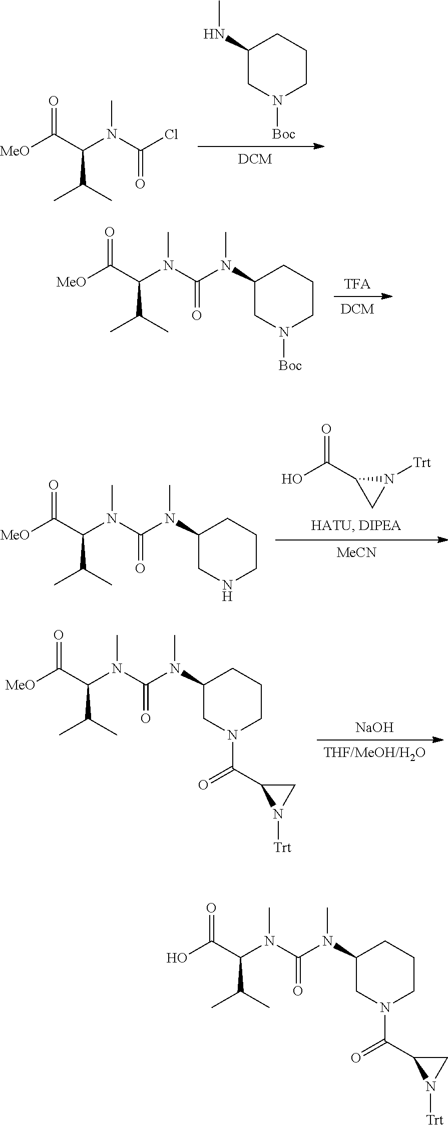

### Step 1: Synthesis of tert-butyl(S)-3-(3-((S)-1-methoxy-3-methyl-1-oxobutan-2-yl)-1,3-dimethylureido) piperidine-1-carboxylate

A mixture of methyl N-(chlorocarbonyl)-N-methyl-L-valinate (1.94 g, 9.34 mmol) in DCM was added to a solution of(S)-tert-butyl 3-(methylamino) piperidine-1-carboxylate (2.80 g, 13.08 mmol) in DCM (18 mL) at 0° C. The mixture was stirred at 40° C. for 3 h. The mixture was added to saturated aq. NH₄Cl (80 mL), and the aqueous phase was extracted with DCM (3×40 mL). the combined organic phase was washed with brine (50 mL), dried over Na₂SO₄, filtered and concentrated under reduced pressure. The crude product was purified by silica gel chromatography (30%->100% EtOAc/pet. ether) to afford the desired product (1.9 g, 55.3% yield). LCMS (ESI) m/z: [M+H] calculated for C₁₉H₃₅N₃O₅: 386.26; found 386.2.

### Step 2: Synthesis of methyl N-methyl-N-(methyl ((S)-piperidin-3-yl) carbamoyl)-L-valinate

To a solution of give tert-butyl(S)-3-(3-((S)-1-methoxy-3-methyl-1-oxobutan-2-yl)-1,3-dimethylureido) piperidine-1-carboxylate (1 g, 2.59 mmol) in DCM (10 mL) was added TFA (3.84 mL, 51.88 mmol) at 0° C. The reaction was stirred at room temperature for 1 h. The mixture was added into saturated aq. Na₂CO₃ (100 mL) at 0° C. to adjust to pH 9. The aqueous phase was extracted with DCM (3×50 mL) and the combined organic phases were washed with brine (10 mL), dried with Na₂SO₄, filtered and concentrated under reduced pressure to afford the desired product (710 mg, crude). LCMS (ESI) m/z: [M+H] calculated for C₁₄H₂₇N₃O₃: 286.21; found 286.1.

### Step 3: Synthesis of methyl N-methyl-N-(methyl ((S)-1-((R)-1-tritylaziridine-2-carbonyl) piperidin-3-yl) carbamoyl)-L-valinate

To a solution of (R)-1-tritylaziridine-2-carboxylic acid (1.24 g, 2.63 mmol, 70% purity) in MeCN (5 mL) at 0° C. was added DIPEA (1.22 mL, 7.01 mmol) and HATU (1.33 g, 3.50 mmol) followed by methyl N-methyl-N-(methyl ((S)-piperidin-3-yl) carbamoyl)-L-valinate (500 mg, 1.75 mmol). The reaction mixture was warmed to room temperature and stirred 30 min. The mixture was added to saturated aq. NH₄Cl (100 mL) and the aqueous phase was extracted with DCM (3×50 mL). The combined organic phase was washed with brine (60 mL), dried over Na₂SO₄, filtered and concentrated under reduced pressure. The residue was purified by silica gel chromatography (50%->100% EtOAc/pet. ether) to afford the desired product (650 mg, 49.7% yield). LCMS (ESI) m/z: [M+H] calculated for C₃₆H₄₄N₄O₄: 597.34; found 597.3.

### Step 4: Synthesis of N-methyl-N-(methyl ((S)-1-((R)-1-tritylaziridine-2-carbonyl) piperidin-3-yl) carbamoyl)-L-valine

NaOH (58.34 mg, 1.46 mmol) was added to a solution of methyl N-methyl-N-(methyl ((S)-1-((R)-1-tritylaziridine-2-carbonyl) piperidin-3-yl) carbamoyl)-L-valinate (640 mg, 857.97 μmol) in THF (4 mL), MeOH (1.3 mL), and H₂O (1.3 mL). The mixture was stirred at room temperature for 20 h. The reaction solution was directly lyophilized to afford the desired product (700 mg, crude). LCMS (ESI) m/z: [M+H] calculated for C₃₅H₄₂N₄O₄: 583.32; found 583.4.

### Intermediate A-79. Synthesis of N-methyl-N-(methyl ((S)-1-((R)-1-tritylaziridine-2-carbonyl) pyrrolidin-3-yl) carbamoyl)-L-valine

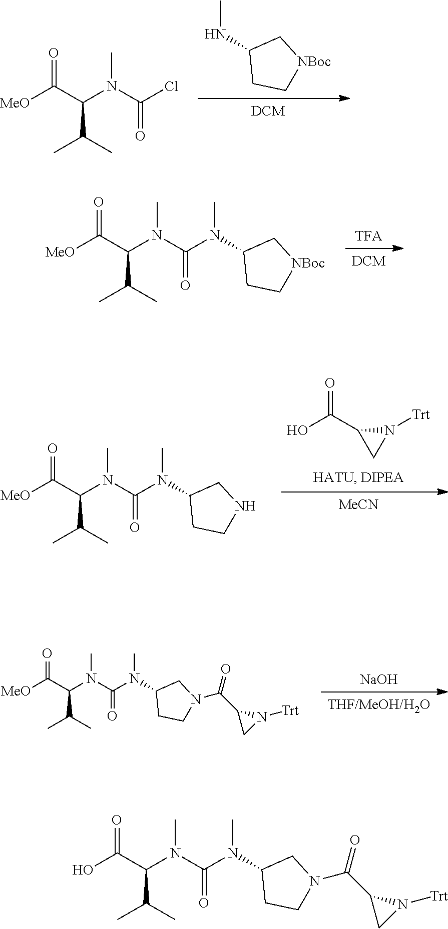

### Step 1: Synthesis of tert-butyl(S)-3-(3-((S)-1-methoxy-3-methyl-1-oxobutan-2-yl)-1,3-dimethylureido) pyrrolidine-1-carboxylate

A solution of methyl N-(chlorocarbonyl)-N-methyl-L-valinate (1.14 g, 5.49 mmol) in DCM (10 mL) was added to a solution of tert-butyl(S)-3-(methylamino) pyrrolidine-1-carboxylate (1.54 g, 7.69 mmol) in DCM (10 mL) at 0° C. The mixture was warmed to room temperature and stirred for 2 h. The mixture was then added to sat. NH₄Cl (50 mL), and the aqueous phase was extracted with DCM (3×30 mL). The combined organic phase was washed with brine (30 mL), dried over Na₂SO₄, filtered and concentrated under reduced pressure. The crude product was purified by silica gel chromatography (30%->100% EtOAc/pet. ether) to afford the desired product (1.07 g, 52.5% yield).

### Step 2: Synthesis of methyl N-methyl-N-(methyl ((S)-pyrrolidin-3-yl) carbamoyl)-L-valinate

To a solution of tert-butyl(S)-3-(3-((S)-1-methoxy-3-methyl-1-oxobutan-2-yl)-1,3-dimethylureido) pyrrolidine-1-carboxylate (1.05 g, 2.83 mmol) in DCM (11 mL) at 0° C. was added TFA (4.19 mL, 56.53 mmol). The reaction was then warmed to room temperature and stirred for 1 h. The mixture was added to sat. Na₂CO₃ (200 mL) at 0° C. dropwise to adjust to pH 9. The aqueous phase was extracted with DCM (3×100 mL), and the combined organic phase was washed with brine (100 mL), dried with Na₂SO₄, filtered and concentrated under reduced pressure to afford the desired product (800 mg, crude).

### Step 3: Synthesis of methyl N-methyl-N-(methyl ((S)-1-((R)-1-tritylaziridine-2-carbonyl) pyrrolidin-3-yl) carbamoyl)-L-valinate

To a solution of (R)-1-tritylaziridine-2-carboxylic acid (1.04 g, 2.21 mmol) in MeCN (4 mL) at 0° C. was added HATU (1.12 g, 2.95 mmol), and DIPEA (1.03 mL, 5.90 mmol) followed by methyl N-methyl-N-(methyl ((S)-pyrrolidin-3-yl) carbamoyl)-L-valinate (400 mg, 1.47 mmol). The mixture was warmed to room temperature and stirred for 0.5 h. The mixture was poured into NH₄Cl aq. (50 mL) and extracted with DCM (3×20 mL). The combined organic phases were washed with brine (30 mL), dried with Na₂SO₄, filtered and concentrated under reduced pressure. The crude product was purified by silica gel chromatography (50%→100% EtOAc/pet. ether) to afford the desired product (580 mg, 67.5% yield).

### Step 4: Synthesis of N-methyl-N-(methyl ((S)-1-((R)-1-tritylaziridine-2-carbonyl) pyrrolidin-3-yl) carbamoyl)-L-valine

To a solution of methyl N-methyl-N-(methyl ((S)-1-((R)-1-tritylaziridine-2-carbonyl) pyrrolidin-3-yl) carbamoyl)-L-valinate (650 mg, 1.12 mmol) in THF (3.9 mL) and MeOH (1.3 mL) was added a solution of NaOH (89.23 mg, 2.23 mmol) in H₂O (1.3 mL). The mixture was stirred at room temperature for 16 h. The reaction mixture was diluted with H₂O (10 mL) and then lyophilized directly to afford the desired product (700 mg, crude). LCMS (ESI) m/z: [M+H] calcd for C₃₄H₄₀N₄O₄: 569.30; found, 569.4.

### Intermediate A-80. Synthesis of N-methyl-N—((S)-3-methyl-4-((R)-1-tritylaziridine-2-carbonyl) piperazine-1-carbonyl)-L-valine

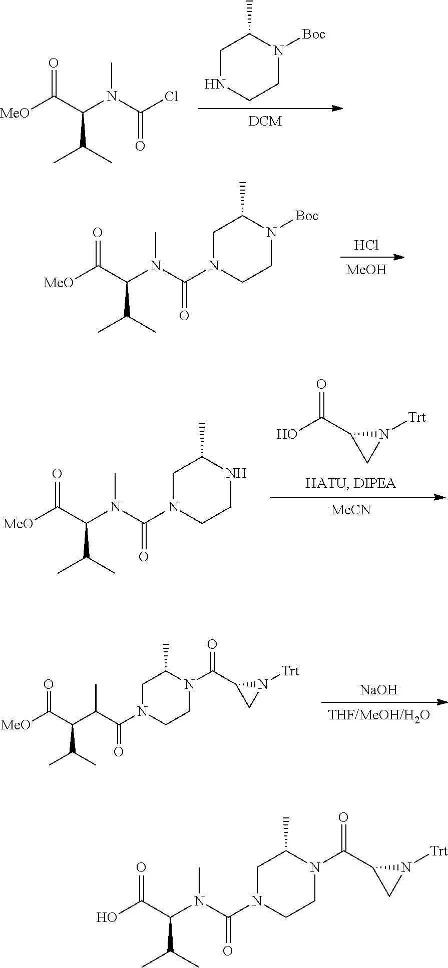

### Step 1: Synthesis of tert-butyl(S)-4-(((S)-1-methoxy-3-methyl-1-oxobutan-2-yl) (methyl) carbamoyl)-2-methylpiperazine-1-carboxylate

To a solution of methyl tert-butyl(S)-2-methylpiperazine-1-carboxylate (3.31 g, 16.52 mmol) in DCM (30 mL) at 0° C. was added a solution of methyl N-(chlorocarbonyl)-N-methyl-L-valinate in DCM (0.55 M, 30 mL). The mixture was stirred at room temperature for 30 min. The reaction mixture was diluted with H₂O (30 mL) and extracted with DCM (3×20 mL). The combined organic layers were washed with brine (2×15 mL), dried over Na₂SO₄, filtered and concentrated under reduced pressure to give a residue. The residue was purified by silica gel chromatography (20%->50% EtOAc/pet. ether) to afford the desired product (5 g, 81.5% yield). LCMS (ESI) m/z: [M+H] calcd for C₁₈H₃₃N₃O₅: 372.2; found 372.1.

### Step 2: Synthesis of methyl N-methyl-N—((S)-3-methylpiperazine-1-carbonyl)-L-valinate

To tert-butyl(S)-4-(((S)-1-methoxy-3-methyl-1-oxobutan-2-yl) (methyl) carbamoyl)-2-methylpiperazine-1-carboxylate (3 g, 8.08 mmol) was added a solution of 4M HCl in MeOH (30 mL). The mixture was stirred at room temperature for 1 h. The reaction mixture was adjusted to pH 8 with saturated aq. NaHCO₃, and was then diluted with H₂O (50 mL) and extracted with DCM (3×30 mL). The combined organic layers were washed with brine (50 mL), dried over Na₂SO₄, filtered and concentrated under reduced pressure to afford the desired product (1.8 g, 82.1% yield).

### Step 3: Synthesis of methyl N-methyl-N—((S)-3-methyl-4-((R)-1-tritylaziridine-2-carbonyl) piperazine-1-carbonyl)-L-valinate

To a solution of (2R)-1-tritylaziridine-2-carboxylic acid (971.10 mg, 2.95 mmol) in MeCN (10 mL) was added HATU (1.35 g, 3.54 mmol), DIPEA (1.54 mL, 8.84 mmol) and methyl N-methyl-N—((S)-3-methylpiperazine-1-carbonyl)-L-valinate (0.8 g, 2.95 mmol). The mixture was stirred at room temperature for 12 h. The reaction mixture was then diluted with H₂O (20 mL) and extracted with DCM (3×15 mL). The combined organic layers were washed with brine 20 mL (2×10 mL), dried over Na₂SO₄, filtered and concentrated under reduced pressure to give a residue. The residue was purified by silica gel chromatography (30%->50% EtOAc/pet. ether) to afford the desired product (0.35 g, 20.4% yield). LCMS (ESI) m/z: [M+H] calculated for C₃₅H₄₂N₄O₄: 583.3; found 583.2.

### Step 4: Synthesis of N-methyl-N—((S)-3-methyl-4-((R)-1-tritylaziridine-2-carbonyl) piperazine-1-carbonyl)-L-valine

To a solution of methyl N-methyl-N—((S)-3-methyl-4-((R)-1-tritylaziridine-2-carbonyl) piperazine-1-carbonyl)-L-valinate (200 mg, 343.21 μmol) in H₂O (1 mL), THF (1 mL), and MeOH (1 mL) at 0° C. was added LiOH·H₂O (14.40 mg, 343.21 μmol). The mixture was stirred at room temperature for 8 h and was then lyophilized directly to afford the desired product (390 mg, 98.7% yield). LCMS (ESI) m/z: [M+Na] calculated for C₃₄H₄₀N₄O₄: 591.3; found 591.2.

### Intermediate A-81. Synthesis of N-methyl-N—((S)-3-methyl-4-((S)-1-tritylaziridine-2-carbonyl) piperazine-1-carbonyl)-L-valine

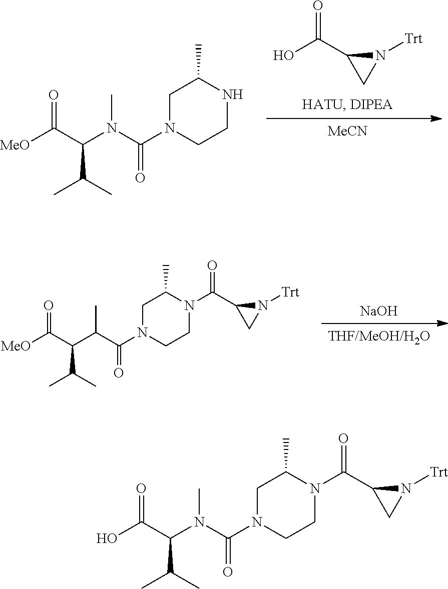

### Step 1: Synthesis of methyl N-methyl-N—((S)-3-methyl-4-((S)-1-tritylaziridine-2-carbonyl) piperazine-1-carbonyl)-L-valinate

To a solution of methyl N-methyl-N—((S)-3-methylpiperazine-1-carbonyl)-L-valinate (500 mg, 1.84 mmol) in MeCN (5 mL) at 0° C. was added(S)-1-tritylaziridine-2-carboxylic acid (1.30 g, 2.76 mmol, 70% purity), HATU (1.05 g, 2.76 mmol) and DIPEA (962.85 μL, 5.53 mmol). The mixture was stirred at room temperature for 30 min. The reaction mixture was then diluted with H₂O (10 mL) and extracted with DCM (3×5 mL). The combined organic layers were washed with brine (2×5 mL), dried over Na₂SO₄, filtered and concentrated under reduced pressure to give a residue. The residue was purified by silica gel chromatography (20%->33% EtOAc/pet. ether) to afford the desired product (0.5 g, 46.6% yield). LCMS (ESI) m/z: [M+Na] calculated for C₃₅H₄₂O₄N₄: 605.2; found 605.2 .

### Step 2: Synthesis of N-methyl-N—((S)-3-methyl-4-((S)-1-tritylaziridine-2-carbonyl) piperazine-1-carbonyl)-L-valine

To a solution of methyl N-methyl-N—((S)-3-methyl-4-((S)-1-tritylaziridine-2-carbonyl) piperazine-1-carbonyl)-L-valinate (400 mg, 686.42 μmol) in H₂O (2 mL), THF (2 mL), and MeOH (2 mL) at 0° C. was added LiOH·H₂O (28.80 mg, 686.42 μmol). The mixture was stirred at room temperature for 8 h. The mixture was lyophilized directly to afford then desired product (390 mg, 98.7% yield). LCMS (ESI) m/z: [M+Na] calculated for C₃₄H₄₀N₄O₄: 591.3; found 591.2.

### Intermediate A-82. Synthesis of N-methyl-N—((R)-3-methyl-4-((R)-1-tritylaziridine-2-carbonyl) piperazine-1-carbonyl)-L-valine

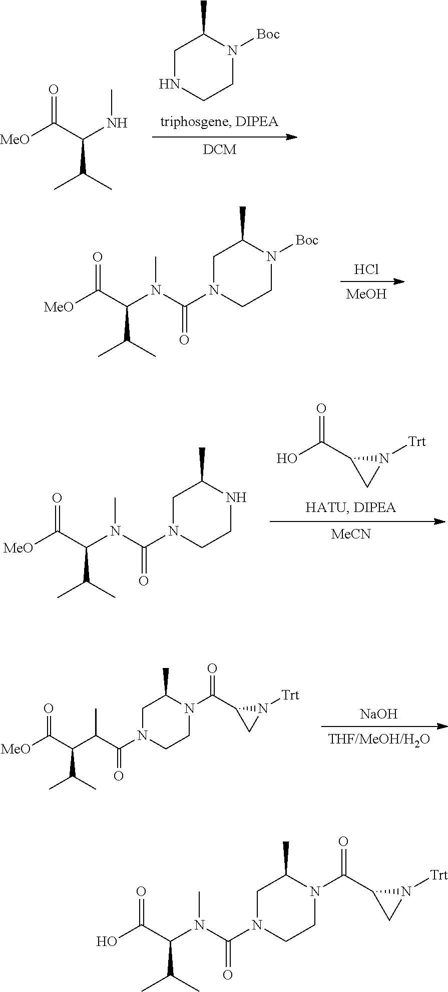

### Step 1: Synthesis of tert-butyl (R)-4-(((S)-1-methoxy-3-methyl-1-oxobutan-2-yl) (methyl) carbamoyl)-2-methylpiperazine-1-carboxylate

To a mixture of methyl methyl-L-valinate hydrochloride (3 g, 16.51 mmol) and DIPEA (17.26 mL, 99.09 mmol) in DCM (60 mL) at 0° C. was added bis (trichloromethyl) carbonate (2.45 g, 8.26 mmol) in one portion. The mixture was stirred at 0° C. for 30 min, then tert-butyl (R)-2-methylpiperazine-1-carboxylate (3.31 g, 16.51 mmol) was added to the mixture. The mixture was stirred at 0° C. for 1 h, then the pH of the solution was adjusted to 8 with sat. NaHCO₃. The residue was poured into H₂O (20 mL) and stirred for 5 min. The aqueous phase was extracted with EtOAc (2×20 mL), and the combined organic phase was washed with sat. NaHCO₃ (20 mL), dried with Na₂SO₄, filtered and concentrated under reduced pressure. The residue was purified by silica gel chromatography (1%->10% EtOAc/pet. ether) to afford the desired product (3.1 g, 50.5% yield). LCMS (ESI) m/z: [M+H] calculated for C₁₈H₃₃N₃O₅: 372.3; found 372.2.

### Step 2: Synthesis of methyl N-methyl-N—((R)-3-methylpiperazine-1-carbonyl)-L-valinate

To a mixture of tert-butyl (R)-4-(((S)-1-methoxy-3-methyl-1-oxobutan-2-yl) (methyl) carbamoyl)-2-methylpiperazine-1-carboxylate (2.5 g, 6.73 mmol) was added 4M HCl in MeOH (25 mL) at 0° C. The mixture was stirred at room temperature for 1 h. The mixture was then concentrated under reduced pressure to afford the desired product (2 g, 96.5% yield).

### Step 3: Synthesis of methyl N-methyl-N—((R)-3-methyl-4-((R)-1-tritylaziridine-2-carbonyl) piperazine-1-carbonyl)-L-valinate

To a mixture of (R)-1-tritylaziridine-2-carboxylic acid (1.03 g, 3.12 mmol) and HATU (1.11 g, 2.92 mmol) in MeCN (1 mL) was added DIPEA (1.36 mL, 7.80 mmol) followed by methyl N-methyl-N—((R)-3-methylpiperazine-1-carbonyl)-L-valinate (600 mg, 1.95 mmol). The mixture was stirred at room temperature for 1 h. The mixture was then concentrated under reduced pressure. The residue was purified by silica gel chromatography (1%->50% EtOAc/pet. ether) to afford the desired product (450 mg, 39.62% yield). LCMS (ESI) m/z: [M+H] calculated for C₃₅H₄₂N₄O₄: 583.3; found 583.2.

### Step 4: Synthesis of N-methyl-N—((R)-3-methyl-4-((R)-1-tritylaziridine-2-carbonyl) piperazine-1-carbonyl)-L-valine

To a mixture of methyl N-methyl-N—((R)-3-methyl-4-((R)-1-tritylaziridine-2-carbonyl) piperazine-1-carbonyl)-L-valinate (450 mg, 772.23 μmol) in H₂O (1 mL), MeOH (1 mL), and THF (3 mL) was added LiOH·H₂O (48.60 mg, 1.16 mmol). The mixture was stirred at room temperature for 10 h and was then lyophilized to afford the desired product (410 mg, 92.4% yield). LCMS (ESI) m/z: [M+Na] calculated for C₃₄H₄₀N₄O₄: 591.3; found 591.3.

### Intermediate A-83. Synthesis of N-methyl-N—((R)-3-methyl-4-((S)-1-tritylaziridine-2-carbonyl) piperazine-1-carbonyl)-L-valine

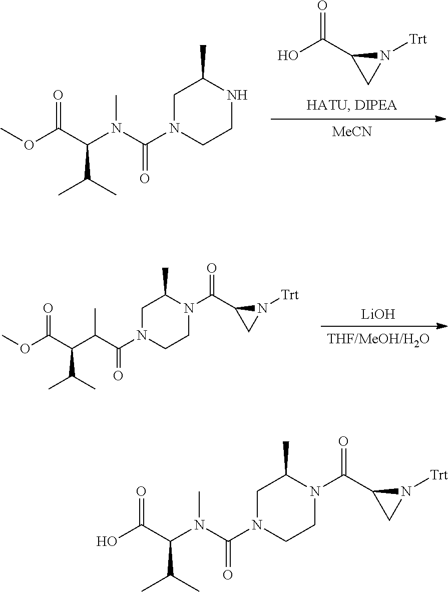

### Step 1: Synthesis of methyl N-methyl-N—((R)-3-methyl-4-((S)-1-tritylaziridine-2-carbonyl) piperazine-1-carbonyl)-L-valinate

To a mixture of(S)-1-tritylaziridine-2-carboxylic acid (1.03 g, 3.12 mmol) and HATU (1.11 g, 2.92 mmol) in MeCN (1 mL) was added DIPEA (1.36 mL, 7.80 mmol) followed by methyl N-methyl-N—((R)-3-methylpiperazine-1-carbonyl)-L-valinate (600 mg, 1.95 mmol). The mixture was stirred at room temperature for 1 h and was then concentrated under reduced pressure. The residue was purified by silica gel chromatography (0%->50% EtOAc/pet. ether) to afford the desired product (430 mg, 37.8% yield). LCMS (ESI) m/z: [M+H] calculated for C₃₅H₄₂N₄O₄: 583.3; found 583.2 Step 2: Synthesis of N-methyl-N—((R)-3-methyl-4-((S)-1-tritylaziridine-2-carbonyl) piperazine-1-carbonyl)-L-valine

To a mixture of methyl N-methyl-N—((R)-3-methyl-4-((S)-1-tritylaziridine-2-carbonyl) piperazine-1-carbonyl)-L-valinate (430 mg, 737.91 μmol) in H₂O (1 mL), MeOH (1 mL), and THF (3 mL) was added LiOH·H₂O (46.44 mg, 1.11 mmol). The mixture was stirred at room temperature for 10 h and was then lyophilized to afford the desired product (370 mg, 87.3% yield). LCMS (ESI) m/z: [M+Na] calculated for C₃₄H₄₀N₄O₄: 591.3; found 591.3.

### Intermediate A-84. Synthesis of N—((R)-4-(tert-butoxycarbonyl)-2-methylpiperazine-1-carbonyl)-N-methyl-L-valine

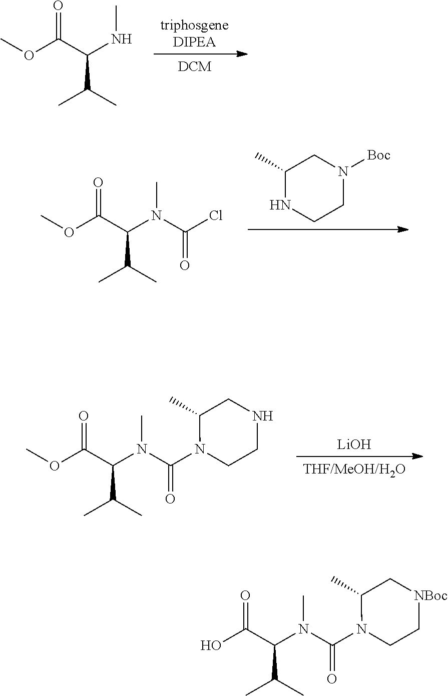

### Step 1: Synthesis of methyl N-(chlorocarbonyl)-N-methyl-L-valinate

To a solution of methyl methyl-L-valinate hydrochloride (1.8 g, 9.91 mmol) in DCM (20 mL) at 0° C. was added DIPEA (5.18 mL, 29.73 mmol) followed by bis (trichloromethyl) carbonate (1.47 g, 4.95 mmol). The mixture was stirred at 0° C. for 20 min. The reaction mixture used for the next step directly without workup.

### Step 2: Synthesis of tert-butyl (R)-4-(((S)-1-methoxy-3-methyl-1-oxobutan-2-yl) (methyl) carbamoyl)-3-methylpiperazine-1-carboxylate

A solution of methyl N-(chlorocarbonyl)-N-methyl-L-valinate (1.03 g, 4.96 mmol) in DCM (10 mL) was added to a solution of tert-butyl (3R)-3-methylpiperazine-1-carboxylate (993.41 mg, 4.96 mmol) in DCM (1 mL) at 0° C. The mixture was then stirred at 0° C. for 15 min. The mixture was added to aq. NH₄Cl (10 mL) and the solution was then extracted with DCM (3×10 mL). The combined organic phase was washed with brine (2 mL), dried over Na₂SO₄, filtered and concentrated under reduced pressure. The residue was purified by silica gel chromatography (0%->50% EtOAc/pet. ether) to afford the desired product (750 mg, 36.2% yield).

### Step 3: Synthesis of N—((R)-4-(tert-butoxycarbonyl)-2-methylpiperazine-1-carbonyl)-N-methyl-L-valine

To a solution of tert-butyl (R)-4-(((S)-1-methoxy-3-methyl-1-oxobutan-2-yl) (methyl) carbamoyl)-3-methylpiperazine-1-carboxylate (700 mg, 1.88 mmol) in THF (0.5 mL) and H₂O (0.5 mL) at 0° C. was added LiOH·H₂O (237.23 mg, 5.65 mmol). The mixture was stirred at room temperature for 3 h. The pH of the reaction mixture was adjusted to 6-7 with 1 N HCl. The mixture was extracted with EtOAc (3×10 mL), dried over Na₂SO₄, and concentrated under reduced pressure to afford the desired product (600 mg, 85.5% yield).

### Intermediate A-85. Synthesis of(S)-3-methyl-2-((R)-2-oxo-3-((S)-1-tritylaziridine-2-carboxamido) pyrrolidin-1-yl) butanoic acid

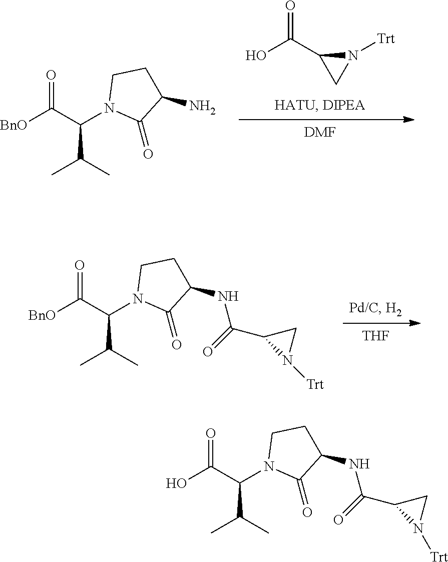

### Step 1: Synthesis of benzyl(S)-3-methyl-2-((R)-2-oxo-3-((S)-1-tritylaziridine-2-carboxamido) pyrrolidin-1-yl) butanoate

To a mixture of benzyl (2S)-2-[(3R)-3-amino-2-oxopyrrolidin-1-yl]-3-methylbutanoate (420.0 mg, 1.446 mmol), DIPEA (934.73 mg, 7.232 mmol) and (2S)-1-(triphenylmethyl) aziridine-2-carboxylic acid (619.40 mg, 1.880 mmol) in DMF (5 mL) at 0° C. was added HATU (659.99 mg, 1.736 mmol). The resulting mixture was stirred at room temperature for 2 h. The reaction mixture was quenched with H₂O. The resulting mixture was extracted with EtOAc (2×10 mL), and the combined organic layers were washed with brine (10 mL), dried over Na₂SO₄, filtered, and concentrated under reduced pressure. The residue was purified by silica gel column chromatography (30% EtOAc/pet. ether) to afford the desired product (480 mg, 55.2% yield). LCMS (ESI) m/z: [M−H]⁻ calcd for C₃₈H₃₈N₃O₄: 600.29; found 600.3.

### Step 2: Synthesis of(S)-3-methyl-2-((R)-2-oxo-3-((S)-1-tritylaziridine-2-carboxamido) pyrrolidin-1-yl) butanoic acid

A suspension of benzyl (2S)-3-methyl-2-[(3R)-2-oxo-3-[(2S)-1-(triphenylmethyl) aziridine-2-amido]pyrrolidin-1-yl]butanoate (450.0 mg, 0.748 mmol) and Pd/C (200 mg) in THF (5 mL) at room temperature was stirred for 3 h under a hydrogen atmosphere. The mixture was then filtered and concentrated under reduced pressure to afford the desired product (400 mg, crude). LCMS (ESI) m/z: [M−H] calcd for C₃₁H₃₂N₃O₄: 510.24; found 510.2.

### Intermediate A-86. Synthesis of(S)-2-(8-(tert-butoxycarbonyl)-1-oxo-2,8-diazaspiro[4.5]decan-2-yl)-3-methylbutanoic acid

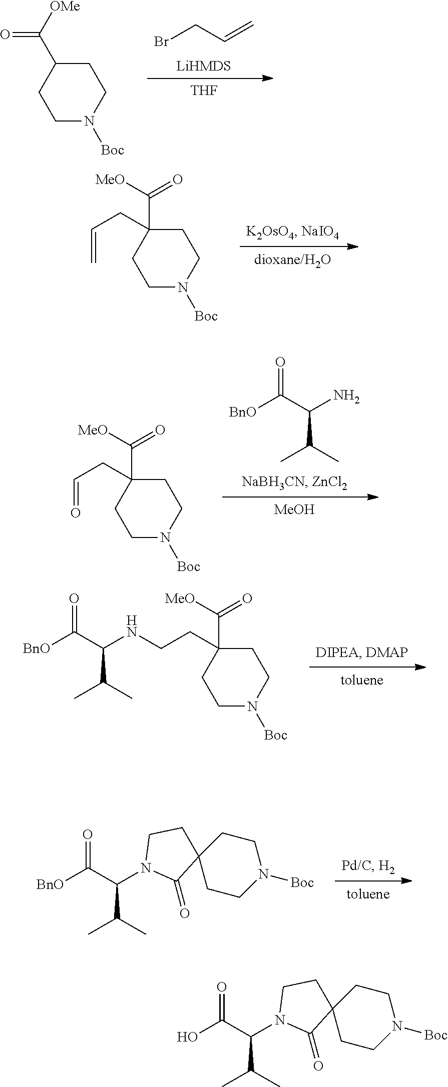

### Step 1: Synthesis of 1-(tert-butyl) 4-methyl 4-allylpiperidine-1,4-dicarboxylate

To a solution of 1-tert-butyl 4-methyl piperidine-1,4-dicarboxylate (5.0 g, 20.551 mmol) in THF (50 mL) at −78° C. was added LiHMDS (27 mL, 26.714 mmol, 1M in THF) followed by allyl bromide (3.23 g, 26.716 mmol). The resulting mixture was stirred overnight at room temperature. The reaction was quenched with sat. aq. NH₄Cl (aq.) and the combined organic layers were washed with brine (3×100 mL), dried over Na₂SO₄, filtered and concentrated under reduced pressure. The residue was purified by silica gel column chromatography (50% EtOAc/pet. ether) to afford the desired product (4.5 g, 73.4% yield).

### Step 2: Synthesis of 1-(tert-butyl) 4-methyl 4-(2-oxoethyl) piperidine-1,4-dicarboxylate

To a solution of 1-tert-butyl 4-methyl 4-(prop-2-en-1-yl) piperidine-1,4-dicarboxylate (1.0 g, 3.529 mmol) and K₂OsO₄·2H₂O (1.3 g, 3.529 mmol) in 1,4-dioxane (5 mL) and H₂O (5 mL) at 0° C. was added NaIO₄ (1.51 g, 7.058 mmol). The resulting mixture was stirred at room temperature for 5 h. The mixture was extracted with EtOAc (3×50 mL) and the combined organic layers were washed with H₂O (3×100 mL), dried over Na₂SO₄, filtered, and concentrated under reduced pressure. The crude product was used in the next step directly without further purification to afford the desired product (800 mg, 75. % yield). LCMS (ESI) m/z: [M−H] calcd for C₁₄H₂₃NO_{5: 284.16}; found 284.0.

### Step 3: Synthesis of 1-(tert-butyl) 4-methyl(S)-4-(2-((1-(benzyloxy)-3-methyl-1-oxobutan-2-yl) amino)ethyl) piperidine-1,4-dicarboxylate

To a solution of 1-tert-butyl 4-methyl 4-(2-oxoethyl) piperidine-1,4-dicarboxylate (4.0 g, 14.018 mmol) and benzyl (2S)-2-amino-3-methylbutanoate (3.49 g, 16.822 mmol) in MeOH (40 mL) at 0° C. was added ZnCl₂ (2.10 g, 15.420 mmol) and NaBH₃CN (1.76 g, 28.037 mmol). The resulting mixture was stirred at room temperature for 2 h. The reaction was quenched with sat. aq. NH₄Cl and the combined organic layers were washed with brine (3×100 mL), dried over Na₂SO₄, filtered, and concentrated under reduced pressure. The residue was purified by silica gel column chromatography (50% EtOAc/pet. ether) to afford the desired product. LCMS (ESI) m/z: [M+H] calcd for C₂₆H₄₀N₂O₆: 477.29; found 477.3.

### Step 4: Synthesis of tert-butyl(S)-2-(1-(benzyloxy)-3-methyl-1-oxobutan-2-yl)-1-oxo-2,8-diazaspiro[4.5]decane-8-carboxylate

To a solution of 1-tert-butyl 4-methyl 4-(prop-2-en-1-yl) piperidine-1,4-dicarboxylate (2.20 g, 4.616 mmol) and DIPEA (5.97 g, 46.159 mmol) in toluene was added DMAP (0.56 g, 4.616 mmol) in portions at 120° C. The resulting mixture was stirred overnight at 120° C. The reaction was cooled to room temperature and quenched with sat. aq. NH₄Cl. The combined organic layers were washed with brine (3×100 mL), dried over Na₂SO₄, filtered, and concentrated under reduced pressure. The residue was purified by Prep-TLC(50% EtOAc/pet. ether) to afford the desired product (1.5 g, 50.2% yield). LCMS (ESI) m/z: [M+H] calcd for C₂₅H₃₆N₂O₅: 445.26; found 445.3.

### Step 5: Synthesis of(S)-2-(8-(tert-butoxycarbonyl)-1-oxo-2,8-diazaspiro[4.5]decan-2-yl)-3-methylbutanoic acid

To a solution of tert-butyl 2-[(2S)-1-(benzyloxy)-3-methyl-1-oxobutan-2-yl]-1-oxo-2,8-diazaspiro[4.5]decane-8-carboxylate (2.40 g, 5.398 mmol) in toluene (25 mL) at room temperature was added Pd/C (2.40 g, 22.552 mmol). The resulting suspension was stirred overnight at room temperature under an H₂ atmosphere. The mixture was concentrated under reduced pressure, filtered, the filter cake washed with EtOAc (3×50 mL), and the filtrate was concentrated under reduced pressure. The residue was purified by silica gel column chromatography (50% EtOAc/pet. ether) to afford the desired product (2.2 g, 72.5% yield). LCMS (ESI) m/z: [M−H] calcd for C₁₈H₃₀N₂O₅: 353.22; found 353.2.

### Intermediate A-87. Synthesis of N-methyl-N-(3-oxo-4-((S)-1-tritylaziridine-2-carbonyl) piperazine-1-carbonyl)-L-valine

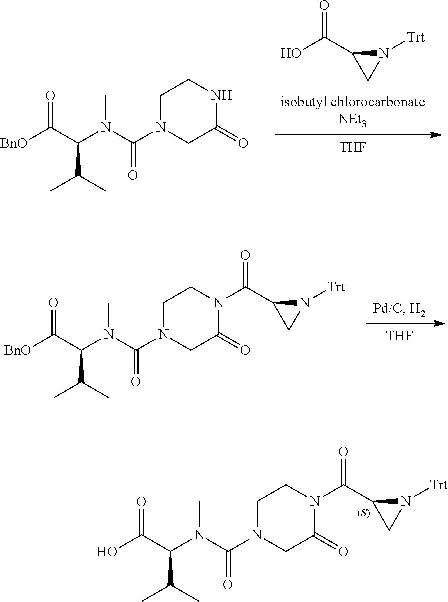

### Step 1: Synthesis of benzyl N-methyl-N-(3-oxo-4-((S)-1-tritylaziridine-2-carbonyl) piperazine-1-carbonyl)-L-valinate

To a solution of (2S)-1-(triphenylmethyl) aziridine-2-carboxylic acid (2.13 g, 6.466 mmol) in THF (10 mL) at 0° C. was added Et₃N (0.87 g, 8.598 mmol) and isobutyl chlorocarbonate (1.44 g, 10.54 mmol). The resulting mixture was stirred at room temperature for 1 h, and then benzyl (2S)-3-methyl-2-[methyl (3-oxopiperazine-1-carbonyl) amino]butanoate (1.50 g, 4.318 mmol) was added. The resulting mixture was stirred overnight at 70° C. The reaction mixture was then concentrated under reduced pressure. The residue was purified by Prep-TLC(50% EtOAc/pet. ether) to afford the desired product (900 mg, 31.6% yield). LCMS (ESI) m/z: [M−H] calcd for C₄₀H₄₂N₄O₅: 657.32; found 657.1.

### Step 2: Synthesis of N-methyl-N-(3-oxo-4-((S)-1-tritylaziridine-2-carbonyl) piperazine-1-carbonyl)-L-valine

A solution of benzyl N-methyl-N-(3-oxo-4-((S)-1-tritylaziridine-2-carbonyl) piperazine-1-carbonyl)-L-valinate (500 mg) and Pd/C (50 mg) in THF (5 mL) was stirred for 2 h at room temperature under a hydrogen atmosphere. The resulting mixture was filtered, the filter cake was washed with MeOH (3×30 mL), and the filtrate was concentrated under reduced pressure to afford the desired product (460 mg, crude). LCMS (ESI) m/z: [M−H] calcd for C₃₃H₃₆N₄O₅: 567.27; found 567.1.

### Intermediate A-88. Synthesis of lithium (R)-1-(3-methoxypropyl) aziridine-2-carboxylate

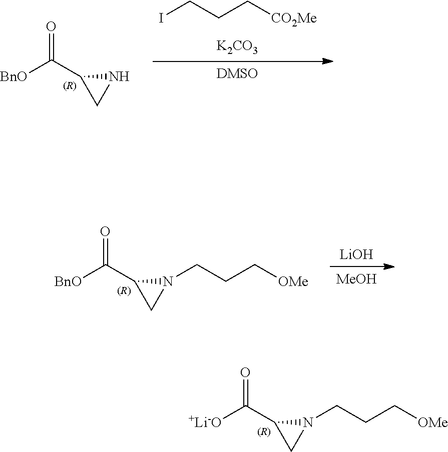

### Step 1: Synthesis of benzyl (R)-1-(3-methoxypropyl) aziridine-2-carboxylate

To a mixture of benzyl (R)-aziridine-2-carboxylate (350.0 mg, 1.975 mmol) and K₂CO₃ (545.95 mg, 3.950 mmol) in DMSO (4 mL) at 60° C. was added 1-iodo-3-methoxypropane (790.13 mg, 3.950 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (30%→38% MeCN/H₂O) to afford the desired product (170 mg, 31.1% yield). LCMS (ESI) m/z: [M+H] calcd for C₁₄H₁₉NO₃: 250.14; found 250.2.

### Step 2: Synthesis of lithium (R)-1-(3-methoxypropyl) aziridine-2-carboxylate

A mixture of benzyl (R)-1-(3-methoxypropyl) aziridine-2-carboxylate (170 mg, 0.682 mmol) and LiOH (57.23 mg, 1.364 mmol) in MeOH (2 mL) was stirred at 0° C. for 1 h. The mixture was concentrated under reduced pressure to afford the desired product (200 mg, crude). LCMS (ESI) m/z: [M+H] calcd for C₇H₁₃NO₃: 160.09; found 160.3.

### Intermediate A-89. Synthesis of lithium(S)-1-(3-methoxypropyl) aziridine-2-carboxylate

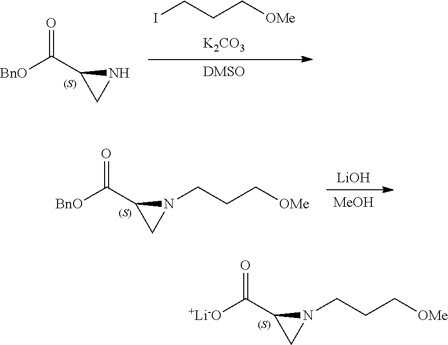

### Step 1: Synthesis of benzyl(S)-1-(3-methoxypropyl) aziridine-2-carboxylate

To a mixture of benzyl(S)-aziridine-2-carboxylate (250 mg, 1.411 mmol) and K₂CO₃ (389.96 mg, 2.822 mmol) in DMSO (4 mL) at 60° C. was added 1-iodo-3-methoxypropane (564.38 mg, 2.822 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (25%→40% H₂O/MeCN) to afford the desired product (234 mg, 63.2% yield). LCMS (ESI) m/z: [M+H] calcd for C₁₄H₁₉NO₃: 250.14; found 250.2.

### Step 2: Synthesis of lithium(S)-1-(3-methoxypropyl) aziridine-2-carboxylate

A mixture of benzyl(S)-1-(3-methoxypropyl) aziridine-2-carboxylate (230 mg, 0.923 mmol) and LiOH·H₂O (77.43 mg, 1.845 mmol) in MeOH (3 mL) was stirred for 1 h at 0° C. The resulting mixture was concentrated under reduced pressure to afford the desired product (320 mg, crude). LCMS (ESI) m/z: [M+H] calcd for C₇H₁₃NO₃: 160.09; found 160.1.

### Intermediate A-90. Synthesis of tert-butyl(S)-2-((S)-1-(benzyloxy)-3-methyl-1-oxobutan-2-yl)-1-oxo-2,7-diazaspiro[4.5]decane-7-carboxylate and tert-butyl (R)-2-((S)-1-(benzyloxy)-3-methyl-1-oxobutan-2-yl)-1-oxo-2,7-diazaspiro[4.5]decane-7-carboxylate

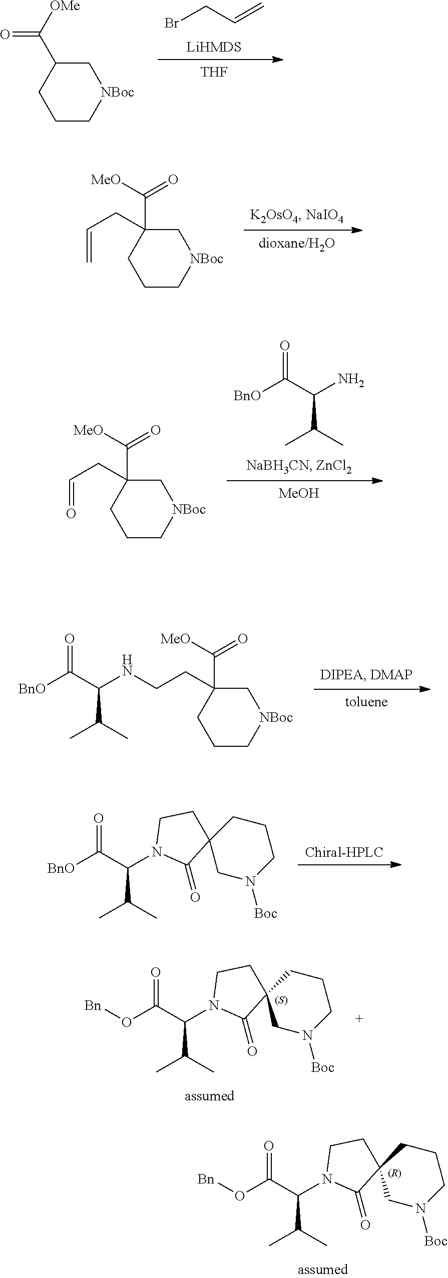

### Step 1: Synthesis of 1-(tert-butyl) 3-methyl 3-allylpiperidine-1,3-dicarboxylate

To a solution of 1-tert-butyl 3-methyl piperidine-1,3-dicarboxylate (10.0 g, 41.101 mmol) and LiHMDS (82 mL, 82.202 mmol, 1M in THF) in THF (100 mL) at −78° C. was added allyl bromide (9.94 g, 82.202 mmol). The reaction was warmed to room temperature and stirred overnight. The solution was then quenched with sat. aq. NH₄Cl and diluted with EtOAc (500 mL). The organic layer was washed with brine (3×150 mL), dried over Na₂SO₄, filtered and concentrated under reduced pressure. The residue was purified by silica gel column chromatography to afford the desired product (9.9 g, 85% yield). LCMS (ESI) m/z: [M+H] calcd for C₁₅H₂₅NO₄: 284.18; found 284.0.

### Step 2: Synthesis of 1-(tert-butyl) 3-methyl 3-(2-oxoethyl) piperidine-1,3-dicarboxylate

To a solution of 1-(tert-butyl) 3-methyl 3-allylpiperidine-1,3-dicarboxylate1-(tert-butyl) 3-methyl 3-allylpiperidine-1,3-dicarboxylate (9.1 g, 32.114 mmol) and 2,6-lutidine (6.88 g, 64.227 mmol) in dioxane (180 mL) and H₂O (180 mL) at 0° C. was added K₂OsO4.2H₂O (591.61 mg, 1.606 mmol). The resulting mixture was stirred for 15 min at room temperature and was then cooled to at 0° C. and NaIO₄ (27.47 g, 128.455 mmol) was added in portions. The resulting mixture was stirred for 3 h at room temperature. And thee reaction was then quenched with sat. aq. Na₂S₂O₃ at 0° C. The resulting mixture was extracted with EtOAc (2×500 mL), and thee combined organic layers were washed with 1M HCl (2×200 mL), brine (2×200 mL), dried over Na₂SO₄, filtered, and concentrated under reduced pressure to afford the desired product (7.5 g, 81.9% yield). LCMS (ESI) m/z: [M+H] calcd for C₁₄H₂₃NO₅: 286.16; found 286.1.

### Step 3: Synthesis of 1-(tert-butyl) 3-methyl 3-(2-(((R)-1-(benzyloxy)-3-methyl-1-oxobutan-2-yl) amino)ethyl) piperidine-1,3-dicarboxylate

To a solution of 1-(tert-butyl) 3-methyl 3-(2-oxoethyl) piperidine-1,3-dicarboxylate (9.0 g, 31.541 mmol) and benzyl (2S)-2-amino-3-methylbutanoate (7.19 g, 34.695 mmol) in MeOH (90 mL) at 0° C. was added ZnCl₂ (4.73 g, 34.695 mmol) and NaBH₃CN (3.96 g, 63.083 mmol). The resulting mixture was stirred overnight at room temperature. Desired product could be detected by LCMS, and it was concentrated under reduced pressure and extracted with EtOAc (1200 mL). The organic layer was washed with brine (3×150 mL), dried over Na₂SO₄, filtered, and concentrated under reduced pressure. Purification by reverse phase chromatography afforded the desired product (9.9 g, 65.9% yield). LCMS (ESI) m/z: [M+H] calcd for C₂₆H₄₀N₂O₆: 477.29; found 477.2.

### Step 4: Synthesis of tert-butyl(S)-2-((S)-1-(benzyloxy)-3-methyl-1-oxobutan-2-yl)-1-oxo-2,7-diazaspiro[4.5]decane-7-carboxylate and tert-butyl (R)-2-((S)-1-(benzyloxy)-3-methyl-1-oxobutan-2-yl)-1-oxo-2,7-diazaspiro[4.5]decane-7-carboxylate

To a solution of 1-(tert-butyl) 3-methyl 3-(2-(((R)-1-(benzyloxy)-3-methyl-1-oxobutan-2-yl) amino)ethyl) piperidine-1,3-dicarboxylate (9.9 g, 20.772 mmol) and DIPEA (26.84 g, 207.715 mmol) in toluene (100 mL) was added DMAP (5.07 g, 41.543 mmol). The resulting mixture was stirred at 80° C. for 50 h. The resulting mixture was concentrated under reduced pressure and the residue was taken up in EtOAc (1000 mL). The organic layer was washed with brine (3×150 mL), dried over Na₂SO₄, filtered, and concentrated under reduced pressure. The crude product was purified by CHIRAL-HPLC(50% EtOH/Hex) to afford tert-butyl(S)-2-((S)-1-(benzyloxy)-3-methyl-1-oxobutan-2-yl)-1-oxo-2,7-diazaspiro[4.5]decane-7-carboxylate (1.75 g) and tert-butyl (R)-2-((S)-1-(benzyloxy)-3-methyl-1-oxobutan-2-yl)-1-oxo-2,7-diazaspiro[4.5]decane-7-carboxylate (1.98 g). LCMS (ESI) m/z: [M+H] calcd for C₂₅H₃₆N₂O₅: 445.26; found 445.2.

Intermediates A-91 and A-92. Synthesis of(S)-2-((S)-7-(tert-butoxycarbonyl)-4-oxo-1-oxa-3,7-diazaspiro[4.4]nonan-3-yl)-3-methylbutanoic acid and(S)-2-((R)-7-(tert-butoxycarbonyl)-4-oxo-1-oxa-3,7-diazaspiro[4.4]nonan-3-yl)-3-methylbutanoic acid

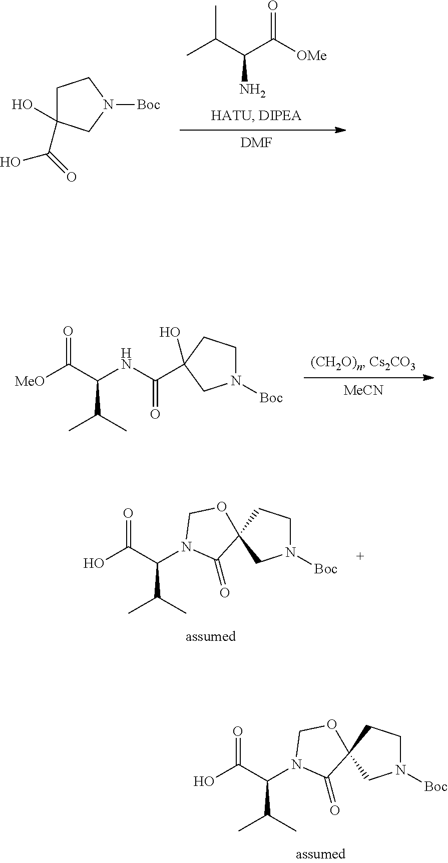

### Step 1: Synthesis of tert-butyl 3-hydroxy-3-(((S)-1-methoxy-3-methyl-1-oxobutan-2-yl) carbamoyl) pyrrolidine-1-carboxylate

To a solution of 1-(tert-butoxycarbonyl)-3-hydroxypyrrolidine-3-carboxylic acid (800 mg, 3.46 mmol) and DIPEA (3.01 mL, 17.3 mmol) in DMF (10 mL) at 0° C. was added methyl L-valinate (681 mg, 5.19 mmol) and HATU (1.71 g, 4.497 mmol). The resulting mixture was warmed to room temperature and stirred for 2 h then diluted with H₂O (20 mL) and extracted into EtOAc (3×20 mL). The combined organic layers were washed with brine (2×20 mL), dried over Na₂SO₄, filtered, and concentrated under reduced pressure. Purification by reverse phase chromatography (30→55% MeCN/H₂O, 0.1% NH₄HCO₃) afforded the desired product (1 g, 76% yield). LCMS (ESI) m/z: [M+Na] calcd for C₁₆H₂₈N₂O₆: 367.18; found 366.9.

### Step 2: Synthesis of(S)-2-((S)-7-(tert-butoxycarbonyl)-4-oxo-1-oxa-3,7-diazaspiro[4.4]nonan-3-yl)-3-methylbutanoic acid and(S)-2-((R)-7-(tert-butoxycarbonyl)-4-oxo-1-oxa-3,7-diazaspiro[4.4]nonan-3-yl)-3-methylbutanoic acid

To a solution of tert-butyl 3-hydroxy-3-(((S)-1-methoxy-3-methyl-1-oxobutan-2-yl) carbamoyl) pyrrolidine-1-carboxylate (1.0 g, 2.90 mmol) and CS₂CO₃ (1.89 g, 5.81 mmol) in MeCN (15 mL) at 0° C. was added paraformaldehyde (436 mg, 14.5 mmol). The resulting mixture was heated to 80° C. and stirred overnight. Purification by reverse phase chromatography (10→40% MeCN/H₂O, 0.1% NH₄HCO₃) afforded a mixture of the desired products. The diastereomers were separated by prep-SFC(30% EtOH/hexanes, 0.3% TFA) to afford(S)-2-((S)-7-(tert-butoxycarbonyl)-4-oxo-1-oxa-3,7-diazaspiro[4.4]nonan-3-yl)-3-methylbutanoic acid (250 mg, 24% yield) and(S)-2-((R)-7-(tert-butoxycarbonyl)-4-oxo-1-oxa-3,7-diazaspiro[4.4]nonan-3-yl)-3-methylbutanoic acid (200 mg, 19% yield). LCMS (ESI) m/z: [M+Na] calcd for C₁₆H₂₆N₂O₆: 365.17; found 365.0.

### Intermediate A-93. Synthesis of(S)-1-((3-methyloxetan-3-yl)methyl) aziridine-2-carboxylic acid

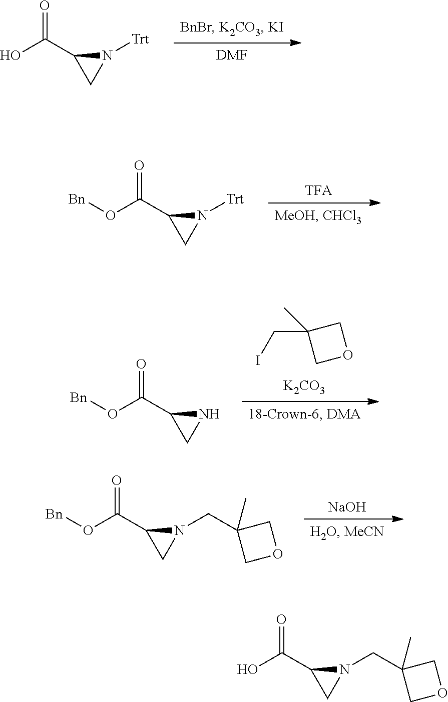

### Step 1: Synthesis of benzyl(S)-1-tritylaziridine-2-carboxylate

To a mixture of(S)-1-tritylaziridine-2-carboxylic acid (3.0 g, 9.11 mmol) and benzyl bromide (2.16 mL, 18.22 mmol) in DMF (30 mL) was added K₂CO₃ (2.25 g, 18.22 mmol) and KI (76 mg, 455 μmol). The reaction mixture was heated to 50° C. and stirred for 30 min then was cooled to room temperature and diluted with H₂O (30 mL) and EtOAc (30 mL). The aqueous layer was extracted with EtOAc (3×40 mL), and the combined organic layers were washed with brine (5×70 mL), dried over Na₂SO₄, filtered, and concentrated under reduced pressure to afford the desired product (4.7 g, crude).

### Step 2: Synthesis of benzyl(S)-aziridine-2-carboxylate

To a mixture of benzyl(S)-1-tritylaziridine-2-carboxylate (3.4 g, 8.10 mmol) in MeOH (17.5 mL) and CHCl₃ (17.5 mL) at 0° C. was added TFA (9.0 mL, 122 mmol). The reaction mixture was stirred for 30 min then was poured into sat. aq. NaHCO₃ (50 mL), extracted into DCM (4×35 mL), dried over Na₂SO₄, filtered, and concentrated under reduced pressure. Purification by silica gel column chromatography (6→100% EtOAc/pet. ether) afforded the desired product (445 mg, 31% yield).

### Step 3: Synthesis of benzyl(S)-1-((3-methyloxetan-3-yl)methyl) aziridine-2-carboxylate

To a mixture of benzyl(S)-aziridine-2-carboxylate (440 mg, 2.48 mmol) and 3-(iodomethyl)-3-methyloxetane (2.11 g, 9.93 mmol) in DMA (5 mL) was added K₂CO₃ (1.72 g, 12.42 mmol) and 18-crown-6 (32.8 mg, 124 μmol). The reaction mixture was heated to 80° C. and stirred for 12 h, and was then was diluted with H₂O (25 mL) and EtOAc (25 mL). The aqueous layer was extracted with EtOAc (3×20 mL), and the combined organic layers were washed with brine (5×45 mL), dried over Na₂SO₄, filtered, and concentrated under reduced pressure. Purification by prep-TLC (50% EtOAc/pet. ether) afforded the desired product (367 mg, 57% yield). LCMS (ESI) m/z: [M+H] calcd for C₁₅H₁₉NO₃: 262.14; found 262.0.

### Step 4: Synthesis of(S)-1-((3-methyloxetan-3-yl)methyl) aziridine-2-carboxylic acid

To a mixture of benzyl(S)-1-((3-methyloxetan-3-yl)methyl) aziridine-2-carboxylate (100 mg, 383 μmol) in MeCN (500 μL) and H₂O (500 μL) at 0° C. was added NaOH (23 mg, 574 μmol). The reaction mixture was stirred at 0° C. for 1 h then was concentrated under reduced pressure to afford the desired product (100 mg, crude). LCMS (ESI) m/z: [M+H] calcd for C₈H₁₃NO₃: 172.10; found 172.0.

### Intermediate A-94. Synthesis of (2R,3R)-1-(tert-butylsulfinyl)-3-ethylaziridine-2-carboxylic acid

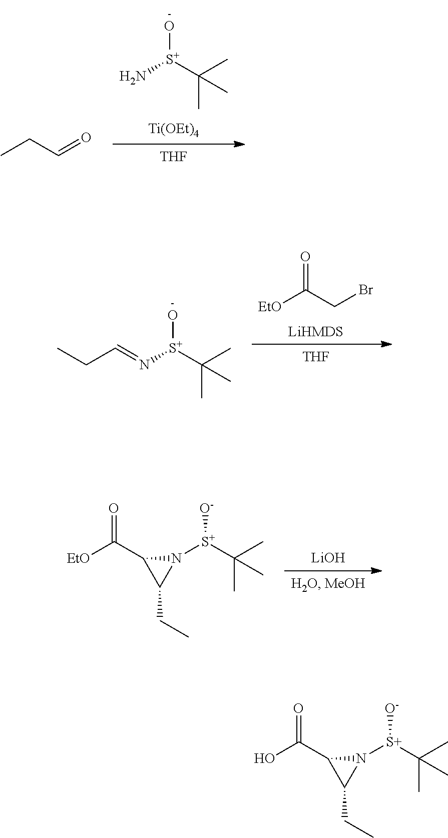

### Step 1: Synthesis of (R,E)-2-methyl-N-propylidenepropane-2-sulfinamide

To a solution of propionaldehyde (6.27 mL, 86.1 mmol) in THF (200 mL) was added (R)-2-methylpropane-2-sulfinamide (10.4 g, 86.1 mmol) and titanium ethoxide (51 mL, 170 mmol). The reaction mixture was heated to 70° C. for 3 h then cooled to room temperature and quenched with H₂O (50 mL), filtered, and extracted into EtOAc (3×30 mL). The combined organic layers were washed with brine (30 mL), dried over Na₂SO₄, filtered, and concentrated under reduced pressure. Purification by silica gel column chromatography (9→17% EtOAc/pet. ether) afforded the desired product (4.0 g, 29% yield).

### Step 2: Synthesis of ethyl (2R,3R)-1-(tert-butylsulfinyl)-3-ethylaziridine-2-carboxylate

To a solution of ethyl 2-bromoacetate (2.74 mL, 24.8 mmol) in THF (40 mL) at −78° C. was added LiHMDS (24.80 mL, 1 M in THF). After 30 min (R,E)-2-methyl-N-propylidenepropane-2-sulfinamide (2.0 g, 12.4 mmol) in THF (20 mL) was added to the reaction mixture. The mixture was stirred for 1 h then warmed to room temperature, quenched with H₂O (50 mL), and extracted into EtOAc (3×50 mL). The combined organic layers were washed with brine (2×50 mL), dried over Na₂SO₄, filtered, and concentrated under reduced pressure. Purification by silica gel column chromatography (17→25% EtOAc/pet. ether) afforded product (1.34 g, 44% yield). LCMS (ESI) m/z: [M+H] calcd for C₁₁H₂₁NO₃S: 248.13; found 248.1.

### Step 3: Synthesis of (2R,3R)-1-(tert-butylsulfinyl)-3-ethylaziridine-2-carboxylic acid

To a solution of ethyl (2R,3R)-1-(tert-butylsulfinyl)-3-ethylaziridine-2-carboxylate (600 mg, 2.4 mmol) in MeOH (3 mL) and H₂O (3 mL) was added LiOH (70 mg, 2.9 mmol). The resulting mixture was stirred for 16 h then diluted with H₂O (20 mL) and washed with DCM (3×10 mL). Lyophilization of the aqueous layer afforded product (600 mg, crude). LCMS (ESI) m/z: [M+H] calcd for C₉H₁₇NO₃S: 220.10; found 220.3.

### Intermediate A-95. Synthesis of (2S,3S)-1-(tert-butylsulfinyl)-3-ethylaziridine-2-carboxylic acid

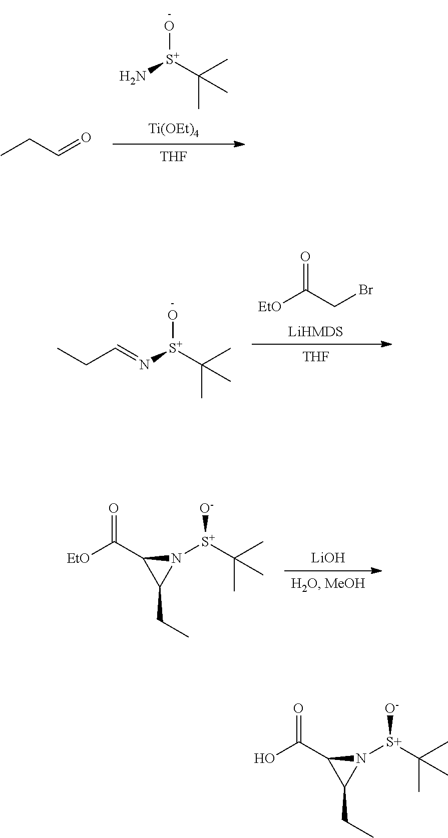

### Step 1: Synthesis of (S,E)-2-methyl-N-propylidenepropane-2-sulfinamide

To a solution of propionaldehyde (6.27 mL, 86.1 mmol) in THF (50 mL) was added(S)-2-methylpropane-2-sulfinamide (10.4 g, 86.1 mmol) and titanium ethoxide (51 mL, 170 mmol). The reaction mixture was heated to 70° C. for 3 h then cooled to room temperature and quenched with H₂O (30 mL), filtered, and extracted into DCM (3×100 mL). The combined organic layers were washed with brine (10 mL), dried over Na₂SO₄, filtered, and concentrated under reduced pressure.

Purification by silica gel column chromatography (25% EtOAc/pet. ether) afforded product (4.6 g, 33% yield).

### Step 2: Synthesis of ethyl (2S,3S)-1-(tert-butylsulfinyl)-3-ethylaziridine-2-carboxylate

To a solution of ethyl 2-bromoacetate (2.74 mL, 24.8 mmol) in THF (40 mL) at −78° C. was added LiHMDS (24.80 mL, 1M in THF). After 30 min (S,E)-2-methyl-N-propylidenepropane-2-sulfinamide (2.0 g, 12.4 mmol) in THF (20 mL) was added to the reaction mixture. The mixture was stirred for 1 h then warmed to room temperature, quenched with H₂O (20 mL), and extracted into EtOAc (3×20 mL). The combined organic layers were washed with brine (2×25 mL), dried over Na₂SO₄, filtered, and concentrated under reduced pressure. Purification by reverse phase chromatography (31→51% MeCN/H₂O, 10 mM NH₄HCO₃) afforded product (600 mg, 20% yield).

LCMS (ESI) m/z: [M+H] calcd for C₁₁H₂₁NO₃S: 248.13; found 248.1.

### Step 3: Synthesis of (2S,3S)-1-(tert-butylsulfinyl)-3-ethylaziridine-2-carboxylic acid

To a solution of ethyl (2S,3S)-1-(tert-butylsulfinyl)-3-ethylaziridine-2-carboxylate (600 mg, 2.4 mmol) in MeOH (300 μL) and H₂O (300 μL) was added LiOH (87 mg, 3.6 mmol). The resulting mixture was stirred for 12 h then diluted with H₂O (20 mL) and washed with DCM (3×10 mL). Lyophilization of the aqueous layer afforded product (600 mg, crude). LCMS (ESI) m/z: [M+H] calcd for C₉H₁₇NO₃S: 220.10; found 220.2.

### Intermediate A-96. Synthesis of (2R,3R)-3-isopropyl-1-tritylaziridine-2-carboxylic acid

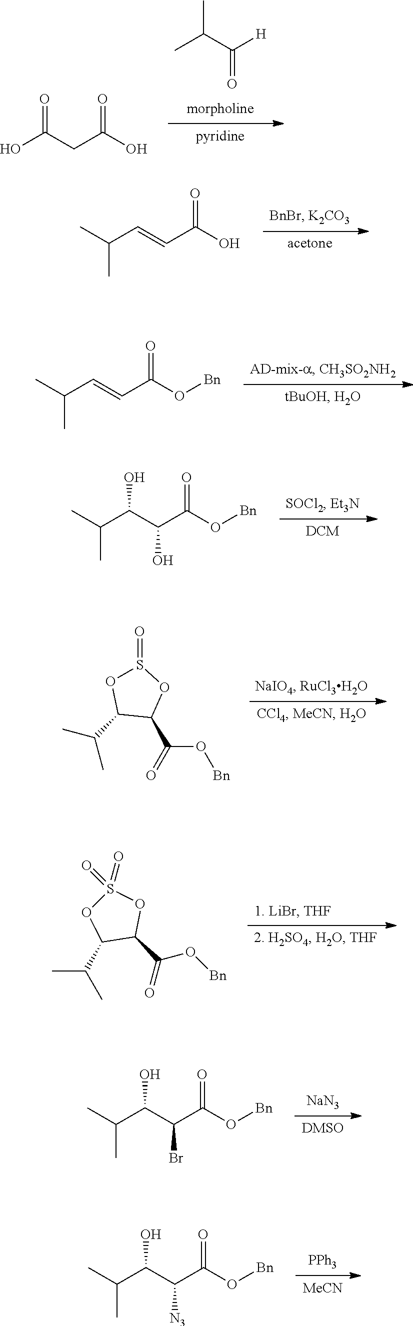

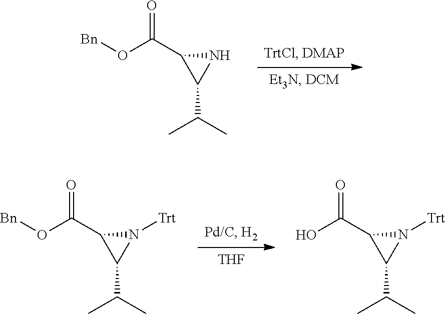

### Step 1: Synthesis of (E)-4-methylpent-2-enoic acid

Two batches of a solution of malonic acid (25.0 mL, 240 mmol), isobutyraldehyde (34.7 mL, 380 mmol) and morpholine (380 μL, 4.32 mmol) in pyridine (75 mL) were stirred for 24 h then were heated to 115° C. and stirred for 12 h. The combined reaction mixtures were poured into H₂SO₄ (1M, 800 mL) and extracted into EtOAc (3×300 mL). The combined organic layers were washed with brine (300 mL), dried over Na₂SO₄, filtered, and concentrated under reduced pressure. The residue was dissolved in NaOH (1 M, 500 mL), washed with EtOAc (2×200 mL), acidified to pH=4-2 with HCl (4M), and extracted into EtOAc (3×300 mL). The combined organic layers were washed with brine (300 mL), dried over Na₂SO₄, filtered, and concentrated under reduced pressure which afforded product (54 g, 98% yield).

### Step 2: Synthesis of benzyl (E)-4-methylpent-2-enoate

To two batches of a solution of (E)-4-methylpent-2-enoic acid (6.25 mL, 52.6 mmol) in acetone (90 mL) was added K₂CO₃ (13.8 g, 100 mmol) and the mixtures were stirred for 30 min. Then a solution of benzyl bromide (6.31 mL, 53.1 mmol) in acetone (10 mL) was added and the mixtures were heated to 75° C. for 5 h. The reaction mixtures were cooled to room temperature and concentrated under reduced pressure. The residue was dissolved in EtOAc (200 mL) and H₂O (200 mL) then extracted into EtOAc (2×200 mL). The combined organic layers were washed with brine (300 mL), dried over Na₂SO₄, filtered, and concentrated under reduced pressure. Purification by silica gel chromatography (0→10% EtOAc/pet. ether) afforded product (9.0 g, 42% yield).

### Step 3: Synthesis of benzyl (2R,3S)-2,3-dihydroxy-4-methylpentanoate

To a solution of AD-mix-α (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.3 g, 79% yield). LCMS (ESI) m/z: [M+Na] calcd for C₁₃H_{1804: 261.11}; found 261.0.

### Step 4: Synthesis of benzyl (4R,5S)-5-isopropyl-1,3,2-dioxathiolane-4-carboxylate 2-oxide

To a solution of benzyl (2R,3S)-2,3-dihydroxy-4-methylpentanoate (10 g, 42.0 mmol) in DCM (100 mL) at 0° C. was added Et₃N (17.5 mL, 126 mmol) and SOCl₂ (4.26 mL, 58.8 mmol). The reaction mixture was stirred 30 min then was diluted with DCM (30 mL) and H₂O (100 mL), extracted into DCM (3×50 mL), washed with brine (100 mL), dried over Na₂SO₄, filtered, and concentrated under reduced pressure which afforded product (11.0 g, 92% yield).

### Step 5: Synthesis of benzyl (4R,5S)-5-isopropyl-1,3,2-dioxathiolane-4-carboxylate 2,2-dioxide

To a solution of benzyl (4R,5S)-5-isopropyl-1,3,2-dioxathiolane-4-carboxylate 2-oxide (11 g, 38.7 mmol) in H₂O (250 mL), MeCN (125 mL), and CCl₄ (125 mL) was added NaIO₄ (3.22 mL, 58.0 mmol) and RuCl₃·H₂O (872 mg, 3.87 mmol). The mixture was stirred at room temperature for 1 h then was diluted with EtOAc (200 mL) and H₂O (50 mL), filtered, and the filtrate was extracted into EtOAc (3×200 mL). The combined organic layers were washed sequentially with brine (200 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11 g, 95% yield).

### Step 6: Synthesis of benzyl (2S,3S)-2-bromo-3-hydroxy-4-methylpentanoate

To a solution of benzyl (4R,5S)-5-isopropyl-1,3,2-dioxathiolane-4-carboxylate 2,2-dioxide (11 g, 36.6 mmol) in THF (520 mL) was added LiBr (3.49 mL, 139 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), extracted into EtOAc (2×300 mL), washed sequentially with Na₂CO₃ (sat. aq., 300 mL) and brine (300 mL), then was concentrated under reduced pressure. Purification by silica gel chromatography (0→17% EtOAc/pet. ether) afforded product (10 g, 81% yield).

### Step 7: Synthesis of benzyl (2R,3S)-2-azido-3-hydroxy-4-methylpentanoate

To a solution of benzyl (2S,3S)-2-bromo-3-hydroxy-4-methylpentanoate (10 g, 33.2 mmol) in DMSO (100 mL) was added NaN₃ (4.32 g, 66.4 mmol). The reaction mixture was stirred at room temperature for 12 h then was diluted with EtOAc (300 mL) and H₂O (200 mL). The aqueous phase was extracted into EtOAc (2×200 mL), washed with brine (200 mL), dried over Na₂SO₄, filtered, and concentrated under reduced pressure. Purification by silica gel chromatography (0→17% EtOAc/pet. ether) afforded product (7.5 g, 79% yield).

### Step 8: Synthesis of benzyl (2R,3R)-3-isopropylaziridine-2-carboxylate

To a solution of benzyl (2R,3S)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 4 h. The reaction mixture was concentrated under reduced pressure and purification by silica gel chromatography (0→17% EtOAc/pet. ether) afforded product (4.5 g, 66% yield). LCMS (ESI) m/z: [M+H] calcd for C₁₃H₁₇NO_{2: 220.13}; found 220.0.

### Step 9: Synthesis of benzyl (2R,3R)-3-isopropyl-1-tritylaziridine-2-carboxylate

To a solution of benzyl (2R,3R)-3-isopropylaziridine-2-carboxylate (2 g, 9.12 mmol) in DCM (30 mL) at 0° C. was added Et₃N (3.81 mL, 27.4 mmol) and trityl chloride (3.05 g, 10.9 mmol) followed by DMAP (111 mg, 912 μmol). The reaction mixture was stirred at 0° C. for 1 h and then was diluted with DCM (50 mL) and H₂O (50 mL) then extracted into DCM (2×30 mL). The combined organic layers were washed with brine (50 mL), dried over Na₂SO₄, filtered, and concentrated under reduced pressure. Purification by silica gel chromatography (0→25% DCM/pet. ether) afforded product (3.1 g, 72% yield).

### Step 10: Synthesis of (2R,3R)-3-isopropyl-1-tritylaziridine-2-carboxylic acid

Two solutions of benzyl (2R,3R)-3-isopropyl-1-tritylaziridine-2-carboxylate (200 mg, 430 μmol) and Pd/C (100 mg) in THF (4 mL) were stirred for 1 h at room temperature under H₂ atmosphere. The reaction mixtures were combined, filtered, and concentrated under reduced pressure. Purification by silica gel chromatography (0→50% EtOAc/pet. ether) afforded product (160 mg, 51% yield).

### Intermediate A-97. Synthesis of (2S,3S)-1-benzyl-3-isopropylaziridine-2-carboxylic acid

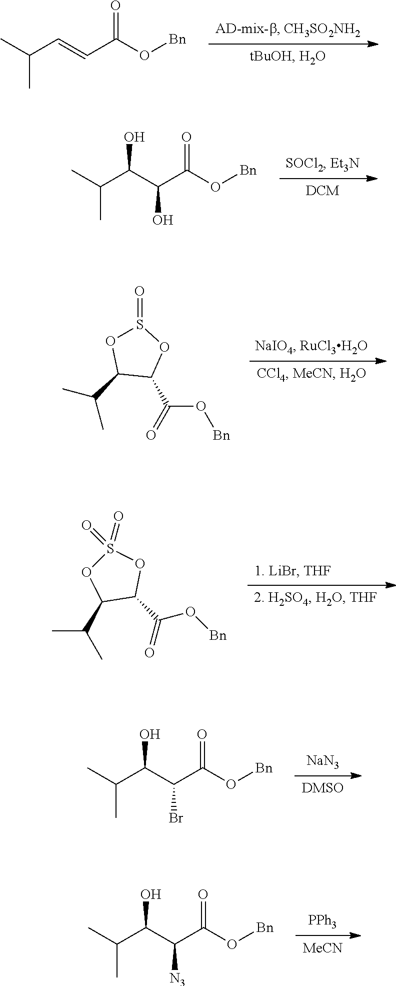

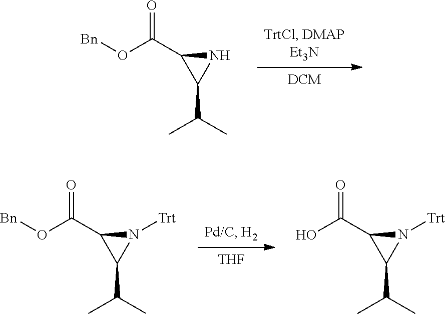

### Step 1: Synthesis of benzyl (2S,3R)-2,3-dihydroxy-4-methylpentanoate

To a solution of AD-mix-β (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.8 g, 84% yield). LCMS (ESI) m/z: [M+Na] calcd for C₁₃H₁₈O₄: 261.11; found 261.0.

### Step 2: Synthesis of benzyl (4S,5R)-5-isopropyl-1,3,2-dioxathiolane-4-carboxylate 2-oxide

To a solution of benzyl (2S,3R)-2,3-dihydroxy-4-methylpentanoate (11.6 g, 48.7 mmol) in DCM (116 mL) at 0° C. was added Et₃N (20.3 mL, 146 mmol) and SOCl₂ (4.94 mL, 68.2 mmol). The reaction mixture was stirred 30 min then was diluted with DCM (100 mL) and H₂O (100 mL), extracted into DCM (3×100 mL), washed with brine (200 mL), dried over Na₂SO₄, filtered, and concentrated under reduced pressure which afforded product (13.0 g, 94% yield).

### Step 3: Synthesis of benzyl (4S,5R)-5-isopropyl-1,3,2-dioxathiolane-4-carboxylate 2,2-dioxide

To a solution of benzyl (4S,5R)-5-isopropyl-1,3,2-dioxathiolane-4-carboxylate 2-oxide (13 g, 45.7 mmol) in H₂O (290 mL), MeCN (145 mL), and CCl₄ (145 mL) was added NaIO₄ (3.80 mL, 68.6 mmol) and RuCl₃·H₂O (1.03 g, 4.57 mmol). The mixture was stirred at room temperature for 1 h then was diluted with DCM (500 mL) and H₂O (300 mL), filtered, and the filtrate was extracted into DCM (3×200 mL). The combined organic layers were washed sequentially with brine (500 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11.5 g, 80% yield).

### Step 4: Synthesis of benzyl (2R,3R)-2-bromo-3-hydroxy-4-methylpentanoate

To a solution of benzyl (4S,5R)-5-isopropyl-1,3,2-dioxathiolane-4-carboxylate 2,2-dioxide (11.5 g, 38.3 mmol) in THF (520 mL) was added LiBr (3.65 mL, 146 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), washed with Na₂CO₃ (sat. aq., 300 mL), then was concentrated under reduced pressure. Purification by silica gel chromatography (0→17% EtOAc/pet. ether) afforded product (10 g, 83% yield).

### Step 5: Synthesis of benzyl (2S,3R)-2-azido-3-hydroxy-4-methylpentanoate

To a solution of benzyl (2R,3R)-2-bromo-3-hydroxy-4-methylpentanoate (10 g, 33.2 mmol) in DMSO (100 mL) was added NaN₃ (4.33 g, 66.6 mmol). The reaction mixture was stirred at room temperature for 12 h then was diluted with EtOAc (300 mL) and H₂O (200 mL). The mixture was extracted into EtOAc (2×200 mL), dried over Na₂SO₄, filtered, and concentrated under reduced pressure. Purification by silica gel chromatography (0→17% EtOAc/pet. ether) afforded product (7.5 g, 76% yield).

### Step 6: Synthesis of benzyl (2S,3S)-3-isopropylaziridine-2-carboxylate

To a solution of benzyl (2S,3R)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 3 h. The reaction mixture was concentrated under reduced pressure and purification by silica gel chromatography (0→17% EtOAc/pet. ether) afforded product (4.5 g, 64% yield). LCMS (ESI) m/z: [M+H] calcd for C₁₃H₁₇NO₂: 220.13; found 220.1.

### Step 7: Synthesis of benzyl (2S,3S)-1-benzyl-3-isopropylaziridine-2-carboxylate

To a solution of benzyl (2S,3S)-3-isopropylaziridine-2-carboxylate (1 g, 4.56 mmol) in MeCN (10 mL) was added K₂CO₃ (3.15 g, 22.8 mmol) and benzyl bromide (812 μL, 6.84 mmol). The reaction mixture was stirred at room temperature for 6 h then was diluted with EtOAc (30 mL) and H₂O (30 mL), extracted into EtOAc (2×30 mL), washed with brine (50 mL), dried over Na₂SO₄, filtered, and concentrated under reduced pressure. Purification by silica gel chromatography (0→17% EtOAc/pet. ether) afforded product (1.3 g, 89% yield). LCMS (ESI) m/z: [M+H] calcd for C₂₀H₂₃NO₂: 310.18; found 310.1.

### Step 8: Synthesis of (2S,3S)-1-benzyl-3-isopropylaziridine-2-carboxylic acid

To a solution of benzyl (2S,3S)-1-benzyl-3-isopropylaziridine-2-carboxylate (600 mg, 1.94 mmol) in THF (6 mL), MeCN (3 mL), and H₂O (6 mL) at 0° C. was added LiOH·H₂O (163 mg, 3.88 mmol). The reaction mixture was stirred at room temperature for 1 h and was adjusted to pH=7-8 with HCl (0.5M). Lyophilization afforded product (750 mg, crude). LCMS (ESI) m/z: [M+H] calcd for C₁₃H₁₇NO₂: 220.13; found 220.1.

### Intermediate A-98, A-99, A-100, and A-101. Synthesis of ethyl (2R,3R)-1-benzhydryl-3-(oxetan-3-yl) aziridine-2-carboxylate, ethyl (2S,3S)-1-benzhydryl-3-(oxetan-3-yl) aziridine-2-carboxylate, ethyl (2R,3S)-1-benzhydryl-3-(oxetan-3-yl) aziridine-2-carboxylate, and ethyl (2S,3R)-1-benzhydryl-3-(oxetan-3-yl) aziridine-2-carboxylate

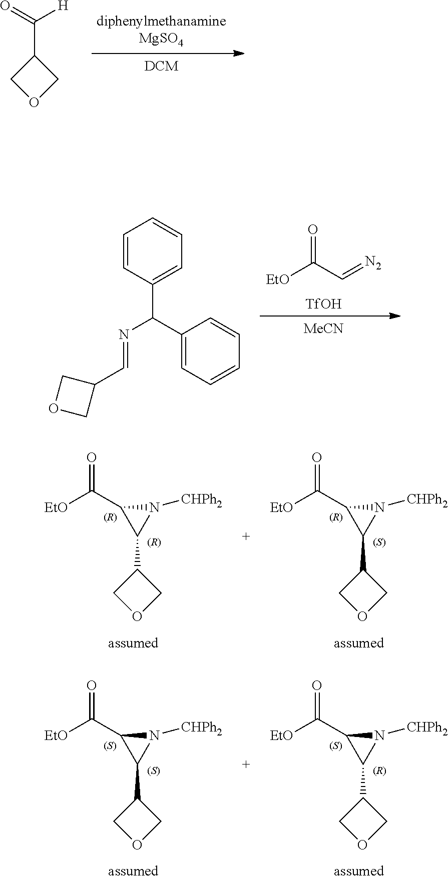

### Step 1: Synthesis of N-benzhydryl-1-(oxetan-3-yl) methanimine

To a solution oxetane-3-carbaldehyde (5.0 g, 58 mmol) and MgSO₄ (6.99 g, 58.1 mmol) in DCM (120 mL) at 0° C. was added diphenylmethanamine (12.1 mL, 69.7 mmol). The mixture was stirred for 12 h at room temperature then filtered and concentrated under reduced pressure to afford the desired compound (14 g, 95.9% yield) which was used without further purification.

### Step 2: Synthesis of ethyl cis-1-benzhydryl-3-(oxetan-3-yl) aziridine-2-carboxylate and ethyl trans-1-benzhydryl-3-(oxetan-3-yl) aziridine-2-carboxylate

To a solution of N-benzhydryl-1-(oxetan-3-yl) methanimine (10 g, 39.79 mmol) in MeCN (150 mL) was added TfOH (878 mL, 9.95 mmol) and after 5 min ethyl diazoacetate (5.0 mL, 47.8 mmol) was added. The reaction mixture was stirred for 12 h at room temperature then cooled to 0° C. and quenched by the addition of saturated NaHCO₃ (300 mL). The aqueous layer was extracted with EtOAc (3×200 mL) and the combined organic layers were washed with brine, dried with Na₂SO₄, filtered, and concentrated under reduced pressure. Purification by reverse phase chromatography (50→65% MeCN/H₂O, 10 mM NH₄HCO₃) afforded racemic ethyl cis-1-benzhydryl-3-(oxetan-3-yl) aziridine-2-carboxylate (1.1 g, 8.2% yield) and racemic ethyl trans-1-benzhydryl-3-(oxetan-3-yl) aziridine-2-carboxylate (780 mg, 5.8% yield)

### Step 3: Separation of racemic ethyl cis-1-benzhydryl-3-(oxetan-3-yl) aziridine-2-carboxylate: ethyl (2R,3R)-1-benzhydryl-3-(oxetan-3-yl) aziridine-2-carboxylate and ethyl (2S,3S)-1-benzhydryl-3-(oxetan-3-yl) aziridine-2-carboxylate

Racemic ethyl cis-1-benzhydryl-3-(oxetan-3-yl) aziridine-2-carboxylate (800 mg, 2.37 mmol) was separated by chiral prep-SFC(25% MeOH/CO₂) to afford ethyl (2R,3R)-1-benzhydryl-3-(oxetan-3-yl) aziridine-2-carboxylate (320 mg, 40% yield) and ethyl (2S,3S)-1-benzhydryl-3-(oxetan-3-yl) aziridine-2-carboxylate (320 mg, 40% yield).

### Step 4: Separation of racemic ethyl trans-1-benzhydryl-3-(oxetan-3-yl) aziridine-2-carboxylate: ethyl (2R,3S)-1-benzhydryl-3-(oxetan-3-yl) aziridine-2-carboxylate and ethyl (2S,3R)-1-benzhydryl-3-(oxetan-3-yl) aziridine-2-carboxylate

Racemic ethyl trans1-benzhydryl-3-(oxetan-3-yl) aziridine-2-carboxylate (700 mg, 2.07 mmol) was separated by chiral prep-SFC(25% EtOH, 0.1% NH₄OH/CO₂) to afford ethyl (2R,3S)-1-benzhydryl-3-(oxetan-3-yl) aziridine-2-carboxylate (300 mg, 42% yield) and ethyl (2S,3R)-1-benzhydryl-3-(oxetan-3-yl) aziridine-2-carboxylate (320 mg, 43% yield).

### Intermediate A-102 and A-103. Synthesis of (2R,3R)-1-benzhydryl-3-(oxetan-3-yl) aziridine-2-carboxylic acid and (2S,3S)-1-benzhydryl-3-(oxetan-3-yl) aziridine-2-carboxylic acid

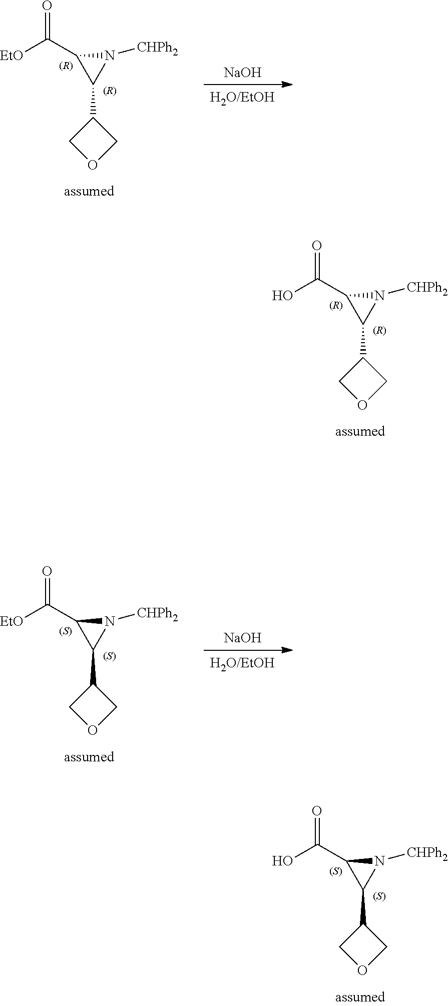

### Step 1: Synthesis of (2R,3R)-1-benzhydryl-3-(oxetan-3-yl) aziridine-2-carboxylic acid

To a solution of ethyl (2R,3R)-1-benzhydryl-3-(oxetan-3-yl) aziridine-2-carboxylate (156 mg, 463 mmol) in EtOH (3 mL) was added 2M NaOH (347 mL, 696 mmol). The reaction mixture was stirred for 3 h at room temperature and then concentrated under reduced pressure. The concentrate was acidified to pH 5 with 1M HCl and extracted with DCM (3×5 mL) and the combined organic layers were washed with brine, dried with Na₂SO₄, filtered and concentrated under reduced pressure to afford the desired compound (110 mg, 72.6% yield).

### Step 2: Synthesis of (2S,3S)-1-benzhydryl-3-(oxetan-3-yl) aziridine-2-carboxylic acid

To a solution of ethyl (2S,3S)-1-benzhydryl-3-(oxetan-3-yl) aziridine-2-carboxylate (150 mg, 444 mmol) in EtOH (5 mL) was added 2M NaOH (333 mL, 666 mmol). The reaction mixture was stirred for 3 h at room temperature and then acidified to pH 5 with 1M HCl. The aqueous layer extracted with DCM (3×10 mL) and the combined organic layers were washed with brine, dried with Na₂SO₄, filtered, and concentrated under reduced pressure to afford the desired compound (120 mg, 86.1% yield).

### Intermediate A-104 and A-105. Synthesis of sodium (2R,3S)-1-benzhydryl-3-(oxetan-3-yl) aziridine-2-carboxylate and sodium (2S,3R)-1-benzhydryl-3-(oxetan-3-yl) aziridine-2-carboxylate

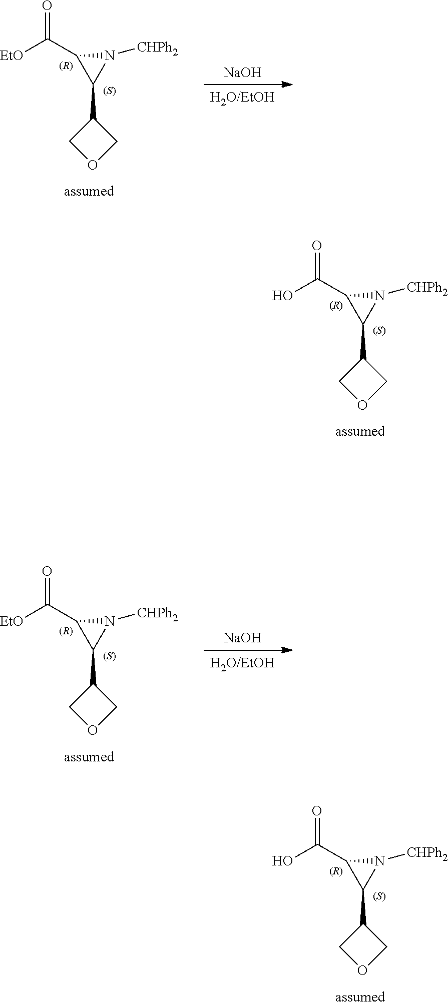

### Step 1: Synthesis of sodium (2R,3S)-1-benzhydryl-3-(oxetan-3-yl) aziridine-2-carboxylate

To a solution of ethyl (2R,3S)-1-benzhydryl-3-(oxetan-3-yl) aziridine-2-carboxylate (150 mg, 444 mmol) in EtOH (3 mL) was added 2M NaOH (333.42 mL, 666 mmol). The reaction mixture was stirred for 3 h at room temperature and then the pH was adjusted to pH 8 with 1M HCl. The resulting solution was lyophilized to afford the desired compound (165 mg, crude) which was used without further purification. LCMS (ESI) m/z: [M] calcd for C₁₉H₁₈NO₃: 308.13; found 308.0.

### Step 2: Synthesis of sodium (2S,3R)-1-benzhydryl-3-(oxetan-3-yl) aziridine-2-carboxylate

To a solution of ethyl (2S,3R)-1-benzhydryl-3-(oxetan-3-yl) aziridine-2-carboxylate (170 mg, 503 mmol) in EtOH (3 mL) was added 2M NaOH (378 mL, 754 mmol). The reaction mixture was stirred for 3 h at room temperature and then the pH was adjusted to pH 8 with 1M HCl. The resulting solution was lyophilized to afford the desired compound (230 mg, crude) which was used without further purification. LCMS (ESI) m/z: [M] calcd for C₁₉H₁₈NO₃: 308.13; found 308.0.

### Intermediate A-106. Synthesis of (R)-1-((benzyloxy) carbonyl)-2-methylaziridine-2-carboxylic acid

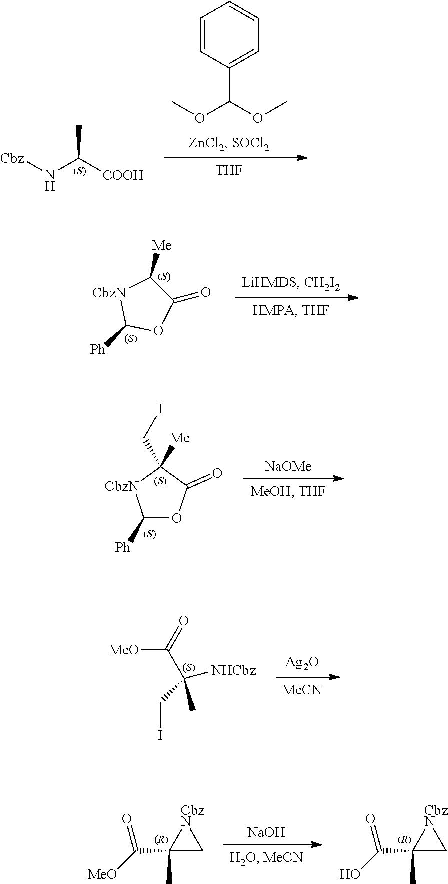

### Step 1: Synthesis of benzyl (2S,4S)-4-methyl-5-oxo-2-phenyloxazolidine-3-carboxylate

To a mixture of ((benzyloxy) carbonyl)-L-alanine (25 g, 111.99 mmol) and (dimethoxymethyl)benzene (71.38 mL, 115.35 mmol) in THF (180 mL) was added SOCl₂ (8.94 g, 123.19 mmol) in one portion at 0° C. The mixture was stirred for 10 min before ZnCl₂ (5.77 mL, 123.26 mmol) was added to the solution, then the mixture was stirred at 0° C. for 4 h. The reaction mixture was quenched by dropwise addition of cold H₂O and adjusted to pH=5 with sat. NaHCO₃, then extracted with EtOAc (2×100 mL). The organic phase was washed with a aq. sat. NaHCO₃ (30 mL) and brine (30 mL), dried over Na₂SO₄, and concentrated under reduced pressure. The residue was purified by silica gel column chromatography (1→10% EtOAc/pet. ether) to afford product (15 g, 43% yield).

### Step 2: Synthesis of benzyl (2S,4S)-4-(iodomethyl)-4-methyl-5-oxo-2-phenyloxazolidine-3-carboxylate

HMPA (5.22 mL, 29.74 mmol) and LHMDS (1 M, 6.62 mL) were mixed in THF (45 mL) under N₂ atmosphere at 20° C. This solution was cooled to −78° C. and a solution of benzyl (2S,4S)-4-methyl-5-oxo-2-phenyloxazolidine-3-carboxylate (2.0 g, 6.42 mmol) in THF (12 mL) was added dropwise with stirring. After stirring an additional 30 min, a solution of CH₂₁₂ (1.55 mL, 19.27 mmol) in THF (6 mL) was added dropwise. The mixture was stirred at −78° C. for 90 min. The mixture was warmed to 0° C. and quenched with sat. aq. NH₄Cl (70 mL). The mixture was extracted with EtOAc (2×30 mL), and the combined organic layers was washed with sat. aq. NH₄Cl (20 mL), H₂O (2×20 mL), and brine (30 mL) dried over Na₂SO₄, filtered, and concentrated under reduced pressure. The resulting residue was purified by silica gel column chromatography (1→20% EtOAc/pet. ether) to afford product (1.2 g, 41.4% yield).

### Step 3: Synthesis of methyl(S)-2-(((benzyloxy) carbonyl) amino)-3-iodo-2-methylpropanoate

To a mixture of benzyl (2S,4S)-4-(iodomethyl)-4-methyl-5-oxo-2-phenyloxazolidine-3-carboxylate (1.2 g, 2.66 mmol) in THF (20 mL) was added a solution of NaOMe (957.69 mg, 5.32 mmol, 30% purity) in MeOH (9 mL) dropwise over 10 min at −40° C. under N₂. The mixture was stirred at −40° C. for 2 h, then warmed to −20° C. and stirred for 1 h. The reaction was quenched by addition of H₂O (20 mL), and the resulting mixture was extracted with EtOAc (3×20 mL). The combined organic layers were washed with brine (20 mL), dried over Na₂SO₄, filtered, and concentrated under reduced pressure. The residue was purified by silica gel column chromatography (1→20% EtOAc/pet. ether) to afford product (870 mg, 2.24 mmol, 84.4% yield).

### Step 4: Synthesis of 1-benzyl 2-methyl (R)-2-methylaziridine-1,2-dicarboxylate

To a mixture of methyl(S)-2-(((benzyloxy) carbonyl) amino)-3-iodo-2-methylpropanoate (0.87 g, 2.31 mmol) in MeCN (125 mL) was added Ag₂O (1.60 g, 6.92 mmol) in one portion at room temperature. The mixture was stirred at 90° C. for 30 min. The mixture was filtered and concentrated under reduced pressure to afford product (500 mg, 2.01 mmol, 86.9% yield).

### Step 5: Synthesis of 1-benzyl 2-methyl (R)-2-methylaziridine-1,2-dicarboxylate

To a mixture of 1-benzyl 2-methyl (R)-2-methylaziridine-1,2-dicarboxylate (250 mg, 1.0 mmol) in MeCN (2.5 mL) and H₂O (2.5 mL) was added NaOH (40.12 mg, 1.0 mmol) in one portion at 0° C. under N₂. The mixture was stirred at 0° C. for 30 min. The mixture was concentrated under reduced pressure to afford crude product (256 mg, crude). LCMS (ESI) m/z: [M+H] calcd for C₁₂H₁₂NO₄: 234.1; found 234.1.

### Intermediate A-107. Synthesis of(S)-1-((benzyloxy) carbonyl)-2-methylaziridine-2-carboxylic acid

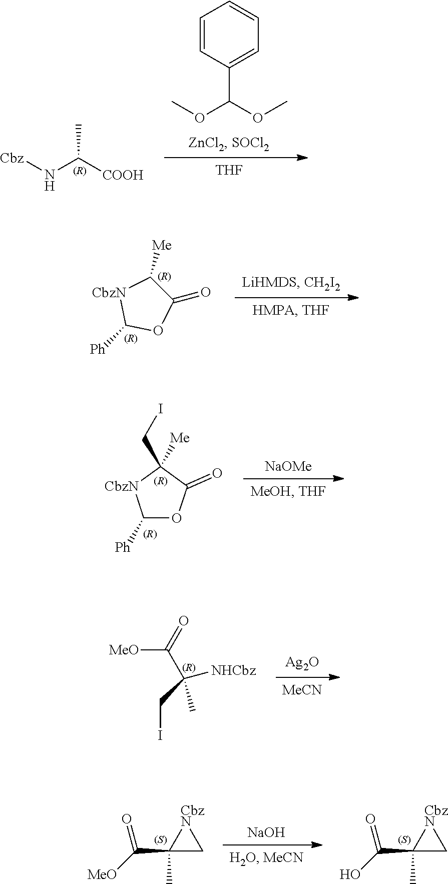

### Step 1: Synthesis of benzyl (2R,4R)-4-methyl-5-oxo-2-phenyloxazolidine-3-carboxylate

Five batches were completed in parallel. To a mixture of ((benzyloxy) carbonyl)-D-alanine (5 g, 22.40 mmol) and (dimethoxymethyl)benzene (3.71 mL, 24.64 mmol) in THF (35 mL) was added SOCl₂ (1.79 mL, 24.64 mmol) in one portion at 0° C. After the mixture was stirred for 10 min, ZnCl₂ (1.15 mL, 24.64 mmol) was added to the solution. Then the mixture was stirred at 0° C. for 4 h. The give batches were combined and concentrated under reduced pressure. The residue was purified by silica gel column chromatography (1→10% EtOAc/pet. ether) to afford product (20 g, 57.4% yield).

### Step 2: Synthesis of benzyl (2R,4R)-4-(iodomethyl)-4-methyl-5-oxo-2-phenyloxazolidine-3-carboxylate

Four batches were completed in parallel. THF (300 mL), HMPA (13.06 mL, 74.34 mmol) and LHMDS (1 M, 16.54 mL) were mixed under N₂ atmosphere at 20° C. with stirring. The solution was cooled to −78° C. and a solution of benzyl (2R,4R)-4-methyl-5-oxo-2-phenyloxazolidine-3-carboxylate (5 g, 16.06 mmol) in THF (84 mL) was added dropwise. After stirring an additional 30 min, a solution of CH₂₁₂ (3.89 mL, 48.18 mmol) in THF (33 mL) was added dropwise. The mixture was stirred at −78° C. for 90 min. The four batches were combined and warmed to 0° C. Sat. aq. NH₄Cl (100 mL) was added to the combined solution and the resulting mixture was extracted with EtOAc (2×100 mL). The combined EtOAc layers was washed with sat. aq. NH₄Cl (50 mL), H₂O (2×20 mL), and brine (30 mL), dried over Na₂SO₄, filtered, and concentrated under reduced pressure. The resulting residue was purified by silica gel column chromatography (1→17% EtOAc/pet. ether) to afford product (16 g, 55.2% yield).

### Step 3: Synthesis of methyl (R)-2-(((benzyloxy) carbonyl) amino)-3-iodo-2-methylpropanoate

To a mixture of benzyl (2R,4R)-4-(iodomethyl)-4-methyl-5-oxo-2-phenyloxazolidine-3-carboxylate (16 g, 35.46 mmol) in THF (90 mL) was added NaOMe (12.77 g, 70.91 mmol, 30% purity) dropwise over 10 min at −40° C. under N₂. The mixture was stirred at −40° C. for 2 h, then warmed to −20° C. and stirred for 1 h. The reaction was quenched by addition of H₂O (100 mL), and the resulting mixture was extracted with diethyl ether (3×100 mL). The combined organic layers were washed with brine (50 mL), dried over Na₂SO₄, filtered, and concentrated under reduced pressure. The resulting residue was purified by silica gel column chromatography (1→17% EtOAc/pet. ether) to afford product (10 g, 74.8% yield.

### Step 4: Synthesis of 1-benzyl 2-methyl(S)-2-methylaziridine-1,2-dicarboxylate

Four batches were completed in parallel. To a mixture of methyl (R)-2-(((benzyloxy) carbonyl) amino)-3-iodo-2-methylpropanoate (8 g, 21.20 mmol) in MeCN (800 mL) was added Ag₂O (14.76 g, 63.64 mmol) in one portion at 20° C. The mixture was stirred at 90° C. for 30 min. The four batches were combined, filtered, and concentrated under reduced pressure to afford product (5.1 g, 90.9% yield.

### Step 5: Synthesis of(S)-1-((benzyloxy) carbonyl)-2-methylaziridine-2-carboxylic acid

To a solution of 1-benzyl 2-methyl(S)-2-methylaziridine-1,2-dicarboxylate (1 g, 4.01 mmol) in MeCN (5 mL) was added a solution of NaOH (240.69 mg, 6.02 mmol) in H₂O (5 mL) at 0° C., then the mixture was stirred at 0° C. for 30 min. The mixture was lyophilized directly to afford crude product (1.05 g, crude). LCMS (ESI) m/z: [M+H] calcd for C₁₂H₁₂NO₄: 234.08; found 234.2.

Intermediates A-108 and A-109. Synthesis of tert-butyl (R)-7-((S)-2-(benzyloxy)-1-cyclopentyl-2-oxoethyl)-6-oxo-2,7-diazaspiro[4.4]nonane-2-carboxylate and tert-butyl(S)-7-((S)-2-(benzyloxy)-1-cyclopentyl-2-oxoethyl)-6-oxo-2,7-diazaspiro[4.4]nonane-2-carboxylate

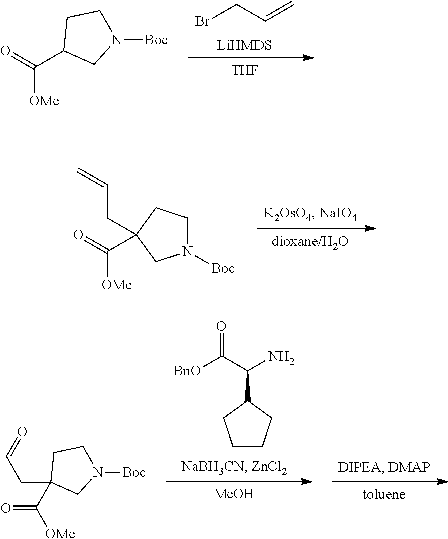

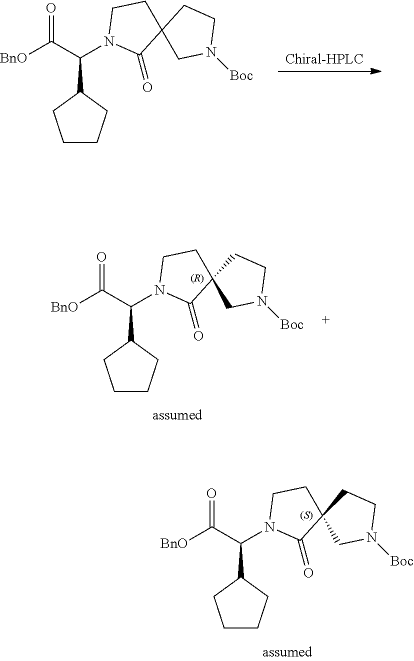

### Step 1: Synthesis of 1-(tert-butyl) 3-methyl 3-allylpyrrolidine-1,3-dicarboxylate

To a solution of 1-(tert-butyl) 3-methyl pyrrolidine-1,3-dicarboxylate (10.0 g, 43.6 mmol) in THF (100 mL) at −78° C. was added LiHMDS (65.0 mL, 65.4 mmol, 1 M in THF). After 1 h allyl bromide (5.63 mL, 65.4 mmol) was added and the resulting mixture was warmed to room temperature overnight. The reaction was quenched at 0° C. by the addition of NH₄Cl (200 mL). The aqueous layer was extracted with EtOAc (3×100 mL) and the combined organic layers were washed with brine, dried with Na₂SO₄, filtered, and concentrated under reduced pressure. Purification by normal phase chromatography (5% EtOAc/pet. ether) afforded the desired product (10.0 g, 76.6% yield).

### Step 3: Synthesis of 1-(tert-butyl) 3-methyl 3-(2-oxoethyl) pyrrolidine-1,3-dicarboxylate

To a solution of 1-(tert-butyl) 3-methyl 3-allylpyrrolidine-1,3-dicarboxylate (10.0 g, 37.1 mmol) and 2,6-dimethylpyridine (8.65 mL, 80.7 mmol) in dioxane (571 mL) and H₂O (142 mL) at 0° C. was added K₂OsO₄·2H₂O (0.27 g, 0.73 mmol). After 15 min NaIO₄ (23.82 g, 111.4 mmol) was added and the resulting mixture was stirred overnight at room temperature and then was diluted with H₂O (200 mL). The aqueous layer extracted with EtOAc (3×200 mL) and the combined organic layers were washed with 2 M HCl, dried with Na₂SO₄, filtered and concentrated under reduced pressure to afford the desired product (9.7 g, crude) which was used without further purification.

### Step 4: Synthesis of 1-(tert-butyl) 3-methyl 3-(2-(((S)-2-(benzyloxy)-1-cyclopentyl-2-oxoethyl) amino) ethyl) pyrrolidine-1,3-dicarboxylate

To a solution of 1-(tert-butyl) 3-methyl 3-(2-oxoethyl) pyrrolidine-1,3-dicarboxylate (9.60 g, 35.4 mmol) in MeOH (100 mL) at 0° C. was added benzyl(S)-2-amino-2-cyclopentylacetate (12.38 g, 53.075 mmol) and zinc chloride (7.23 g, 53.1 mmol). After 30 min NaBH₃CN (4.45 g, 70.8 mmol) was added and the resulting mixture stirred for 2 h at room temperature, concentrated under reduced pressure and the residue diluted with H₂O (150 mL). The aqueous layer was extracted with EtOAc (3×50 mL) and the combined organic layers were washed with brine, dried with Na₂SO₄, filtered, and then concentrated under reduced pressure. Purification by normal phase chromatography (20% EtOAc/pet. ether) afforded the desired product (11.1 g, 64.2% yield). LCMS (ESI) m/z: [M+H] calcd for C₂₇H₄₀N₂O₆: 489.30; found 489.3.

### Step 5: Synthesis of tert-butyl (R)-7-((S)-2-(benzyloxy)-1-cyclopentyl-2-oxoethyl)-6-oxo-2,7-diazaspiro[4.4]nonane-2-carboxylate and tert-butyl(S)-7-((S)-2-(benzyloxy)-1-cyclopentyl-2-oxoethyl)-6-oxo-2,7-diazaspiro[4.4]nonane-2-carboxylate

To a solution of stirred solution of 1-(tert-butyl) 3-methyl 3-(2-(((S)-2-(benzyloxy)-1-cyclopentyl-2-oxoethyl) amino) ethyl) pyrrolidine-1,3-dicarboxylate (11.1 g, 22.7 mmol) in toluene (120 mL) was added DIPEA (39.6 mL, 227 mmol) and DMAP (2.78 g, 22.7 mmol). The resulting mixture was stirred for 2 days at 80° C. and then concentrated under reduced pressure. Purification by reverse phase chromatography (20→70% MeCN/H₂O, 0.1% HCO₂H) afforded a mixture of desired products. The diastereomers were separated by prep-SFC(30% EtOH/CO₂) to afford tert-butyl (R)-7-((S)-2-(benzyloxy)-1-cyclopentyl-2-oxoethyl)-6-oxo-2,7-diazaspiro[4.4]nonane-2-carboxylate (3.73 g, 44.4% yield) LCMS (ESI) m/z: [M+H] calcd for C₂₆H₃₆N₂O₅: 457.27; found 457.3 and tert-butyl(S)-7-((S)-2-(benzyloxy)-1-cyclopentyl-2-oxoethyl)-6-oxo-2,7-diazaspiro[4.4]nonane-2-carboxylate (3.87 g, 46.1% yield)) LCMS (ESI) m/z: [M+H] calcd for C₂₆H₃₆N₂O₅: 457.27; found 457.3.

### Intermediate B-1. Synthesis of N-(3-(3-(4-methoxyphenyl) thioureido) propanoyl)-N-methyl-L-valine

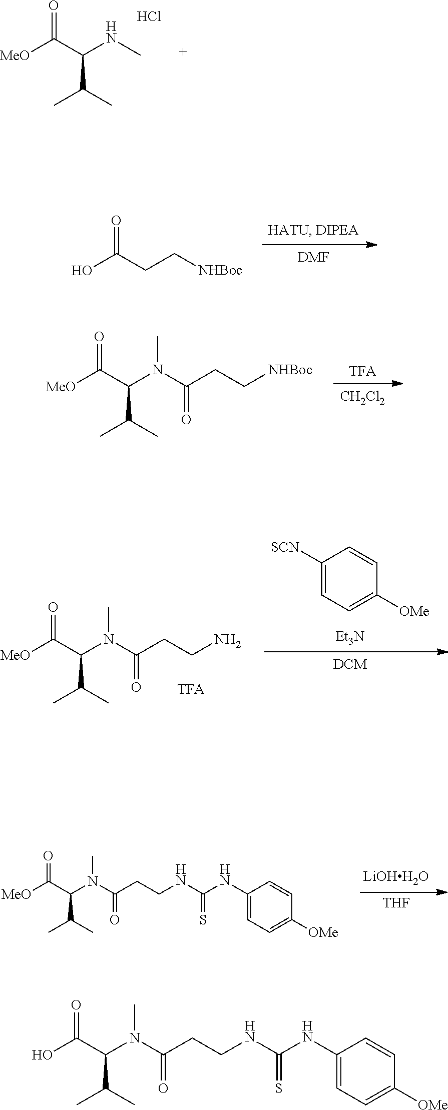

### Step 1: Synthesis of methyl N-(3-((tert-butoxycarbonyl) amino) propanoyl)-N-methyl-L-valinate

To a solution of 3-((tert-butoxycarbonyl) amino) propanoic acid (1.04 g, 5.50 mmol) in DMF (6 mL) was added DIPEA (2.38 mL, 13.7 mmol) followed by HATU (2.71 g, 7.15 mmol). The reaction mixture was stirred for 5 min and methyl methyl-L-valinate hydrochloride (1 g, 5.50 mmol) was added. The reaction was stirred at room temperature for 3 h and was then quenched with H₂O. The aqueous layer was extracted with EtOAc (3×10 mL) and the combined organic layers were washed with brine, and dried over Na₂SO₄, filtered, and concentrated under reduced pressure to afford the desired crude product.

### Step 2: Synthesis of methyl N-(3-aminopropanoyl)-N-methyl-L-valinate trifluoroacetic acid

To a solution of methyl N-(3-((tert-butoxycarbonyl) amino) propanoyl)-N-methyl-L-valinate (1.74 g, 5.50 mmol) in DCM (3 mL) was added TFA (2.09 mL, 27.4 mmol). The reaction was stirred at room temperature overnight and was then concentrated under reduced pressure to afford a solution of the desired crude product as a 33.5% solution in TFA.

### Step 3: Synthesis of methyl N-(3-(3-(4-methoxyphenyl) thioureido) propanoyl)-N-methyl-L-valinate

To a 33.5 wt % solution of methyl N-(3-aminopropanoyl)-N-methyl-L-valinate trifluoroacetic acid (800 mg, 0.811 mmol) in TFA was added DCM (5 mL) followed by Et₃N (593 μL, 4.26 mmol) and 4-methoxyphenyl isothiocyanate (117.0 μL, 852 μmol). The reaction was stirred at room temperature for 3 h. The reaction mixture was then washed with H₂O (2×5 mL), aq. NH₄Cl (5 mL), and brine (5 mL). The organic layer was dried over Na₂SO₄ and concentrated under reduced pressure to afford the crude product (290.2 mg 89.2% yield) as an oil, which was taken on without purification. LCMS (ESI) m/z: [M+H] calcd for C₁₈H₂₇N₃O₄S: 382.18; found 382.2.

### Step 4: Synthesis of N-(3-(3-(4-methoxyphenyl) thioureido) propanoyl)-N-methyl-L-valine

To a solution of methyl N-(3-(3-(4-methoxyphenyl) thioureido) propanoyl)-N-methyl-L-valinate (290.2 mg, 0.76 mmol) in THF (1 mL) was added a solution of LiOH·H₂O (41.4 mg, 0.99 mmol) in H₂O (300 μL). The reaction mixture was stirred overnight and was then acidified with HCl (4 M in dioxane, 120 μL, 0.48 mmol). The solution was then concentrated, the residue was dissolved in EtOAc, and the organic layer washed with H₂O (3×5 mL) and brine (5 mL). The organic layer was dried over Na₂SO₄ and concentrated under reduced pressure to afford the crude product (215.1 mg 77.0% yield), which was taken forward without further purification. LCMS (ESI) m/z: [M+H] calcd for C₁₇H₂₅N₃O₄S: 368.16; found 368.2.

The following table of compounds were prepared using the methods or variations thereof used to synthesize Intermediate B-1.

TABLE 3
Intermediate B

| | Molecular | Calculated | Observed |
|---|---|---|---|
| Structure | Formula | MW | MW |
| 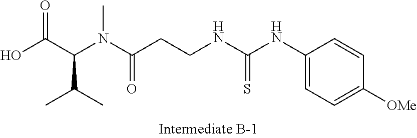 [SMILES: COc1ccc(NC(=S)NCCC(=O)N(C)[C@H](C(=O)O)C(C)C)cc1] | C₁₇H₂₅N₃O₄S | [M + H] = 368.16 | [M + H] = 368.5 |
| 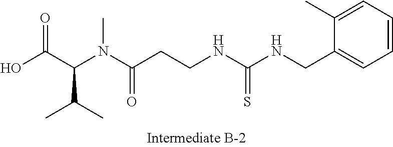 [SMILES: Cc1ccccc1CNC(=S)NCCC(=O)N(C)[C@H](C(=O)O)C(C)C] | C₁₈H₂₇N₃O₃S | [M + H] = 366.19 | [M + H] = 366.6 |
| 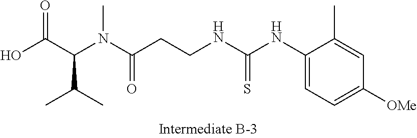 [SMILES: COc1ccc(NC(=S)NCCC(=O)N(C)[C@H](C(=O)O)C(C)C)c(C)c1] | C₁₈H₂₇N₃O₄S | [M + H] = 382.18 | [M + H] = 382.5 |
| 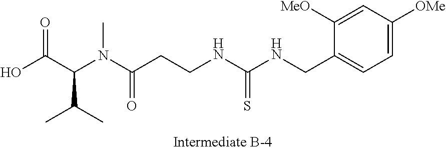 [SMILES: COc1ccc(CNC(=S)NCCC(=O)N(C)[C@H](C(=O)O)C(C)C)c(OC)c1] | C₁₉H₂₉N₃O₅S | [M + H] = 412.19 | [M + H] = 412.6 |
| 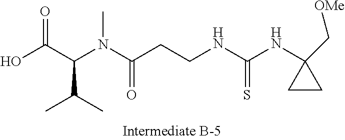 [SMILES: COCC1(NC(=S)NCCC(=O)N(C)[C@H](C(=O)O)C(C)C)CC1] | C₁₅H₂₇N₃O₄S | [M + H] = 346.18 | [M + H] = 346.6 |
| 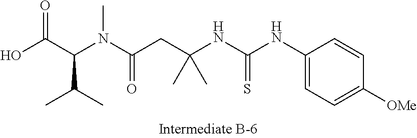 [SMILES: COc1ccc(NC(=S)NC(C)(C)CC(=O)N(C)[C@H](C(=O)O)C(C)C)cc1] | C₁₉H₂₉N₃O₄S | [M + H] = 396.20 | [M + H] = 396.6 |
| 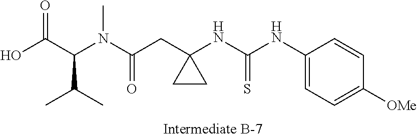 [SMILES: COc1ccc(NC(=S)NC2(CC(=O)N(C)[C@H](C(=O)O)C(C)C)CC2)cc1] | C₁₉H₂₇N₃O₄S | [M + H] = 394.18 | [M + H] = 394.7 |
| 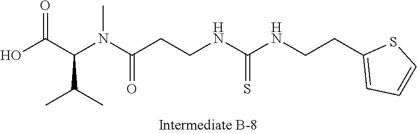 [SMILES: CC(C)[C@@H](C(=O)O)N(C)C(=O)CCNC(=S)NCCc1cccs1] | C₁₆H₂₅N₃O₃S₂ | [M + H] = 372.14 | [M + H] = 327.6 |
| 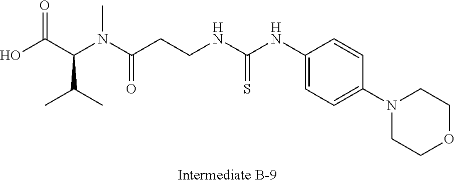 [SMILES: CC(C)[C@@H](C(=O)O)N(C)C(=O)CCNC(=S)Nc1ccc(N2CCOCC2)cc1] | C₂₀H₃₀N₄O₄S | [M + H] = 423.21 | [M + H] = 423.6 |
| 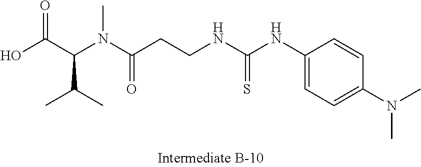 [SMILES: CC(C)[C@@H](C(=O)O)N(C)C(=O)CCNC(=S)Nc1ccc(N(C)C)cc1] | C₁₈H₂₈N₄O₃S | [M + H] = 381.20 | [M + H] = 381.6 |
| 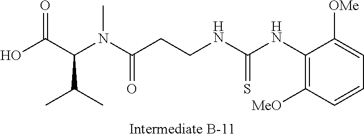 [SMILES: COc1cccc(OC)c1NC(=S)NCCC(=O)N(C)[C@H](C(=O)O)C(C)C] | C₁₈H₂₇N₃O₅S | [M + H] = 398.17 | [M + H] = 398.6 |
| 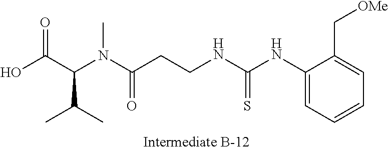 [SMILES: COCc1ccccc1NC(=S)NCCC(=O)N(C)[C@H](C(=O)O)C(C)C] | C₁₈H₂₇N₃O₄S | [M + H] = 382.18 | [M + H] = 382.6 |
| 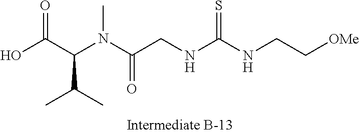 [SMILES: COCCNC(=S)NCC(=O)N(C)[C@H](C(=O)O)C(C)C] | C₁₂H₂₃N₃O₄S | [M + Na] = 328.13 | [M + Na] = 328.5 |
| 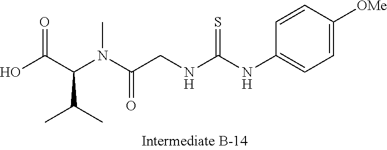 [SMILES: COc1ccc(NC(=S)NCC(=O)N(C)[C@H](C(=O)O)C(C)C)cc1] | C₁₆H₂₃N₃O₄S | [M + H] = 354.15 | [M + H] = 354.5 |
| 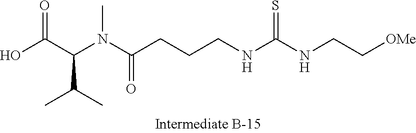 [SMILES: COCCNC(=S)NCCCC(=O)N(C)[C@H](C(=O)O)C(C)C] | C₁₄H₂₇N₃O₄S | [M + H] = 334.18 | [M + H] = 334.6 |
| 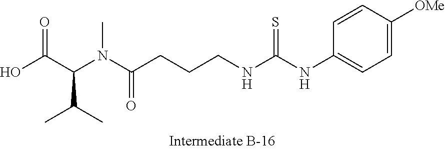 [SMILES: COc1ccc(NC(=S)NCCCC(=O)N(C)[C@H](C(=O)O)C(C)C)cc1] | C₁₈H₂₇N₃O₄S | [M + H] = 382.18 | [M + H] = 382.6 |
| 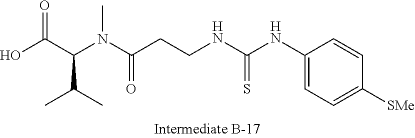 [SMILES: CSc1ccc(NC(=S)NCCC(=O)N(C)[C@H](C(=O)O)C(C)C)cc1] | C₁₇H₂₅N₃O₃S₂ | [M + H] = 384.14 | [M + H] = 384.3 |
| 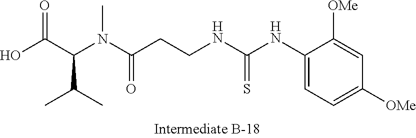 [SMILES: COc1ccc(NC(=S)NCCC(=O)N(C)[C@H](C(=O)O)C(C)C)c(OC)c1] | C₁₈H₂₇N₃O₅S | [M + H] = 398.17 | [M + H] = 398.4 |
| 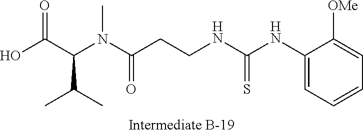 [SMILES: COc1ccccc1NC(=S)NCCC(=O)N(C)[C@H](C(=O)O)C(C)C] | C₁₇H₂₅N₃O₄S | [M + H] = 368.16 | [M + H] = 368.4 |
| 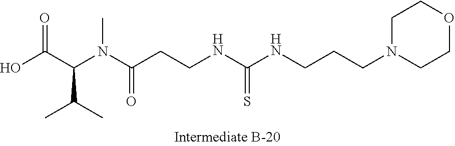 [SMILES: CC(C)[C@@H](C(=O)O)N(C)C(=O)CCNC(=S)NCCCN1CCOCC1] | C₁₇H₃₂N₄O₄S | [M + H] = 389.22 | [M + H] = 389.4 |
| 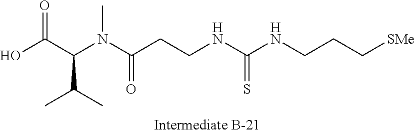 [SMILES: CSCCCNC(=S)NCCC(=O)N(C)[C@H](C(=O)O)C(C)C] | C₁₄H₂₇N₃O₃S₂ | [M + H] = 350.16 | [M + H] = 350.4 |
| 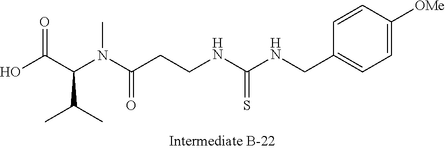 [SMILES: COc1ccc(CNC(=S)NCCC(=O)N(C)[C@H](C(=O)O)C(C)C)cc1] | C₁₈H₂₇N₃O₄S | [M + H] = 382.18 | [M + H] = 382.4 |
| 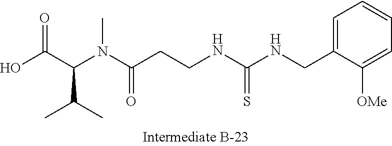 [SMILES: COc1ccccc1CNC(=S)NCCC(=O)N(C)[C@H](C(=O)O)C(C)C] | C₁₈H₂₇N₃O₄S | [M + H] = 382.18 | [M + H] = 382.4 |
| 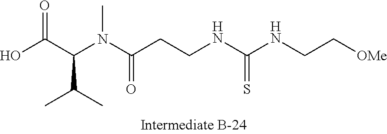 [SMILES: COCCNC(=S)NCCC(=O)N(C)[C@H](C(=O)O)C(C)C] | C₁₃H₂₅N₃O₄S | [M + H] = 320.16 | [M + H] = 320.3 |
| 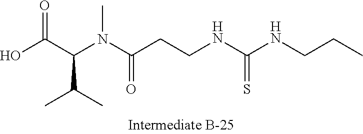 [SMILES: CCCNC(=S)NCCC(=O)N(C)[C@H](C(=O)O)C(C)C] | C₁₃H₂₅N₃O₃S | [M + H] = 304.17 | [M + H] = 304.5 |
| 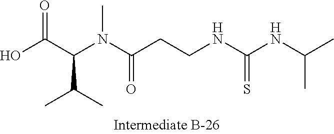 [SMILES: CC(C)NC(=S)NCCC(=O)N(C)[C@H](C(=O)O)C(C)C] | C₁₃H₂₅N₃O₃S | [M + H] = 304.17 | [M + H] = 304.3 |
| 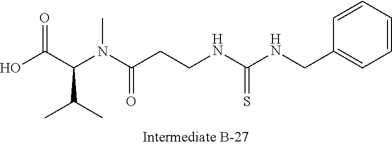 [SMILES: CC(C)[C@@H](C(=O)O)N(C)C(=O)CCNC(=S)NCc1ccccc1] | C₁₇H₂₅N₃O₃S | [M + H] = 352.17 | [M + H] = 352.4 |
| 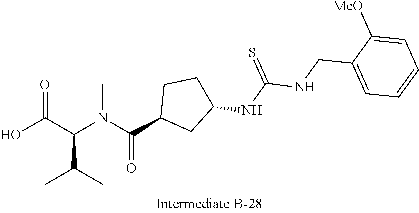 [SMILES: COc1ccccc1CNC(=S)N[C@H]1CC[C@H](C(=O)N(C)[C@H](C(=O)O)C(C)C)C1] | C₂₁H₃₁N₃O₄S | [M + H] = 422.21 | [M + H] = 422.7 |
| 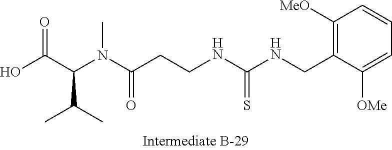 [SMILES: COc1cccc(OC)c1CNC(=S)NCCC(=O)N(C)[C@H](C(=O)O)C(C)C] | C₁₉H₂₉N₃O₅S | [M + H] = 412.19 | [M + H] = 412.2 |
| 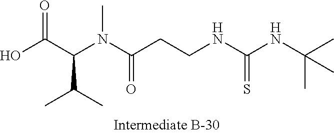 [SMILES: CC(C)[C@@H](C(=O)O)N(C)C(=O)CCNC(=S)NC(C)(C)C] | C₁₄H₂₇N₃O₃S | [M + H] = 318.19 | [M + H] = 318.6 |
| 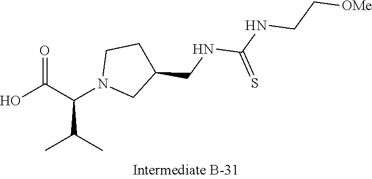 [SMILES: COCCNC(=S)NC[C@@H]1CCN([C@H](C(=O)O)C(C)C)C1] | C₁₄H₂₇N₃O₃S | [M + H] = 318.19 | [M + H] = 318.7 |
| 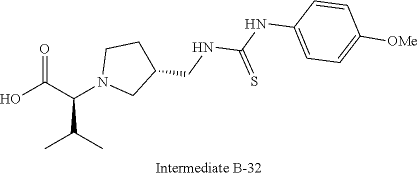 [SMILES: COc1ccc(NC(=S)NC[C@H]2CCN([C@H](C(=O)O)C(C)C)C2)cc1] | C₁₈H₂₇N₃O₃S | [M + H] = 366.19 | [M + H] = 366.7 |
| 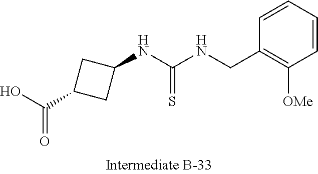 [SMILES: COc1ccccc1CNC(=S)N[C@H]1C[C@H](C(=O)O)C1] | C₁₄H₁₈N₂O₃S | [M + H] = 295.11 | [M + H] = 295.6 |
| 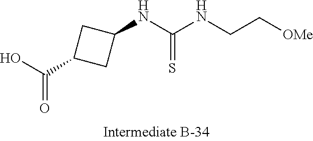 [SMILES: COCCNC(=S)N[C@H]1C[C@H](C(=O)O)C1] | C₉H₁₆N₂O₃S | [M + H] = 233.10 | [M + H] = 233.4 |

### Example 1. Synthesis of (3S)-1-((R)-aziridine-2-carbonyl)-N-((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

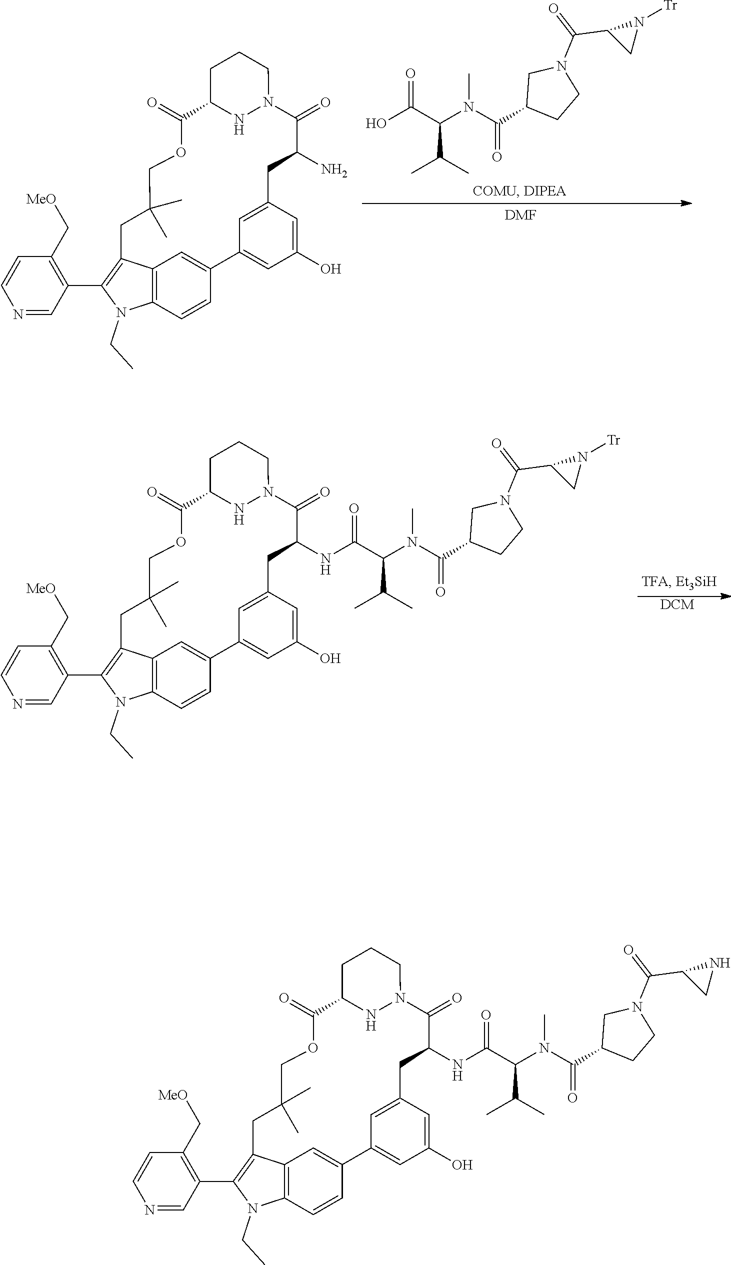

### Step 1: Synthesis of (3S)—N-((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-1-((R)-1-tritylaziridine-2-carbonyl) pyrrolidine-3-carboxamide

To a solution of (6³S,4S)-4-amino-1¹-ethyl-25-hydroxy-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (520.0 mg, 0.831 mmol) and N-methyl-N—((S)-1-((R)-1-tritylaziridine-2-carbonyl) pyrrolidine-3-carbonyl)-L-valine (0.6727 g, 1.25 mmol) in DMF (10 mL) at 0° C. was added COMU (0.5338 mg, 1.25 mmol) followed by DIPEA (1.16 mL, 6.65 mmol). After 2 h, the reaction mixture was extracted with EtOAc (3×100 mL) and the combined organic layers were washed with brine (3×30 mL), dried over Na₂SO₄, filtered, and concentrated under reduced pressure. The crude residue was purified by reverse phase chromatography (10→50% MeCN/H₂O) to afford the desired product (500 mg, 52.4% yield) as a solid. LCMS (ESI) m/z: [M+H] calcd for C₆₉H₇₈N₈O₈: 1147.60; found 1147.8.

### Step 2: Synthesis of (3S)-1-((R)-aziridine-2-carbonyl)-N-((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide

To a stirred solution of (3S)—N-((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-1-((R)-1-tritylaziridine-2-carbonyl) pyrrolidine-3-carboxamide (145.0 mg, 0.126 mmol) in DCM (3 mL) at 0° C. was added Et₃SiH (58.8 mg, 0.505 mmol) followed by TFA (57.6 mg, 0.505 mmol). After 1 h, DIPEA was added to the reaction mixture until pH 8. The resulting mixture was concentrated under reduced pressure, and the residue was purified by reverse phase chromatography (10→50% MeCN/H₂O) to afford the desired product (70 mg, 61.2% yield) as a solid. LCMS (ESI) m/z: [M+H] calcd for C₅₀H₆₄N₈O₈: 905.49; found 905.7.

### Example 7. Synthesis of (2R)—N-(2-(((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

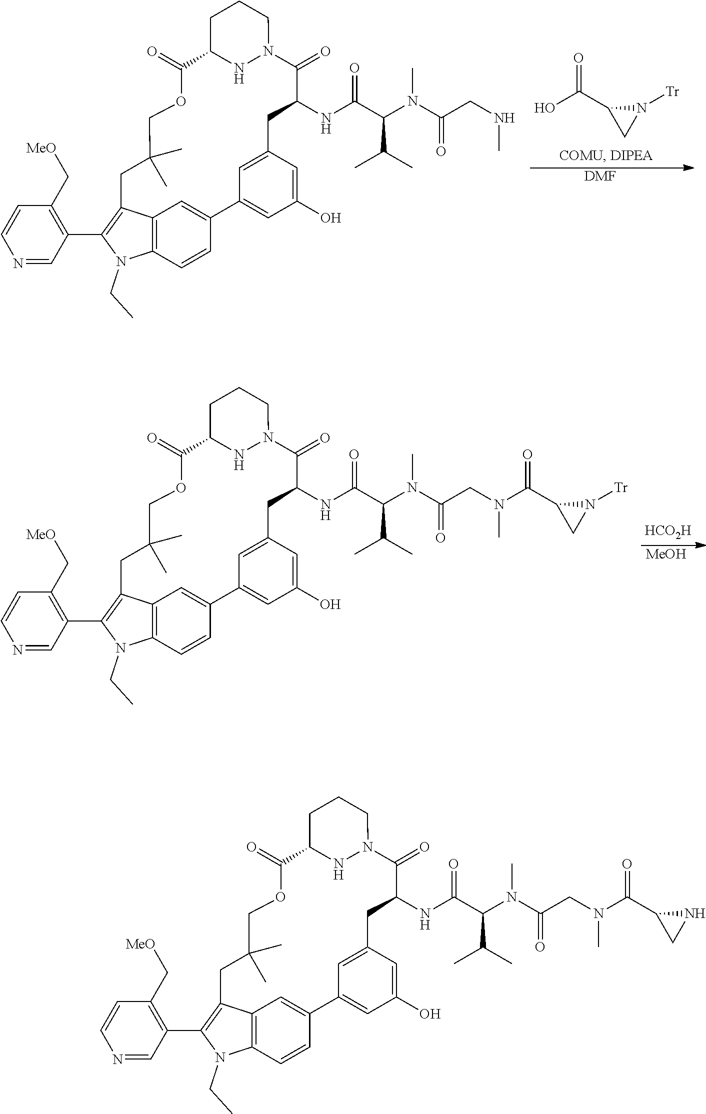

### Step 1: Synthesis of (2R)—N-(2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide

To a solution of (2S)—N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide (285.7 mg, 0.353 mmol) in DMF (3.0 mL) at 0° C. was added (R)-1-tritylaziridine-2-carboxylic acid (232.4 mg, 0.705 mmol) followed by DIPEA (0.61 mL, 4.7 mmol) and COMU (211.4 mg, 0.494 mmol). The resulting mixture was warmed to room temperature and stirred for 1 h. The reaction mixture was diluted with H₂O (15 mL) and the mixture was extracted with EtOAc (3×4 mL). The combined organic layers were washed with brine (10 mL), dried over Na₂SO₄, filtered, and concentrated under reduced pressure. The residue was purified by prep-TLC(12% EtOAc/pet. ether) to afford the desired product (301 mg, 68% yield) as a solid. LCMS (ESI) m/z: [M+H] calcd for C₆₇H₇₆N₈O₈: 1121.59; found 1121.8.

### Step 2: Synthesis of (2R)—N-(2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2R)—N-(2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide (301.0 mg, 0.268 mmol) in MeOH (3.0 mL) at 0° C. was added HCO₂H (1.50 mL). The reaction mixture was stirred for 1 h and then neutralized to pH 8 with DIPEA. The resulting mixture was diluted with H₂O (15 mL) and extracted with EtOAc (3×4 mL). The combined organic layers were dried over Na₂SO₄, filtered, and concentrated under reduced pressure. The residue was purified by reverse phase chromatography (30→60% MeCN/H₂O) to afford the desired product (89.9 mg, 38% yield) as a solid. LCMS (ESI) m/z: [M+H] calcd for C₄₈H₆₂N₈O₈: 879.48; found 879.7.

### Example 15. Synthesis of two isomers, 15A and 15B, of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(3-((((4-methoxyphenyl) imino) methylene) amino)-N-methylpropanamido)-3-methylbutanamide

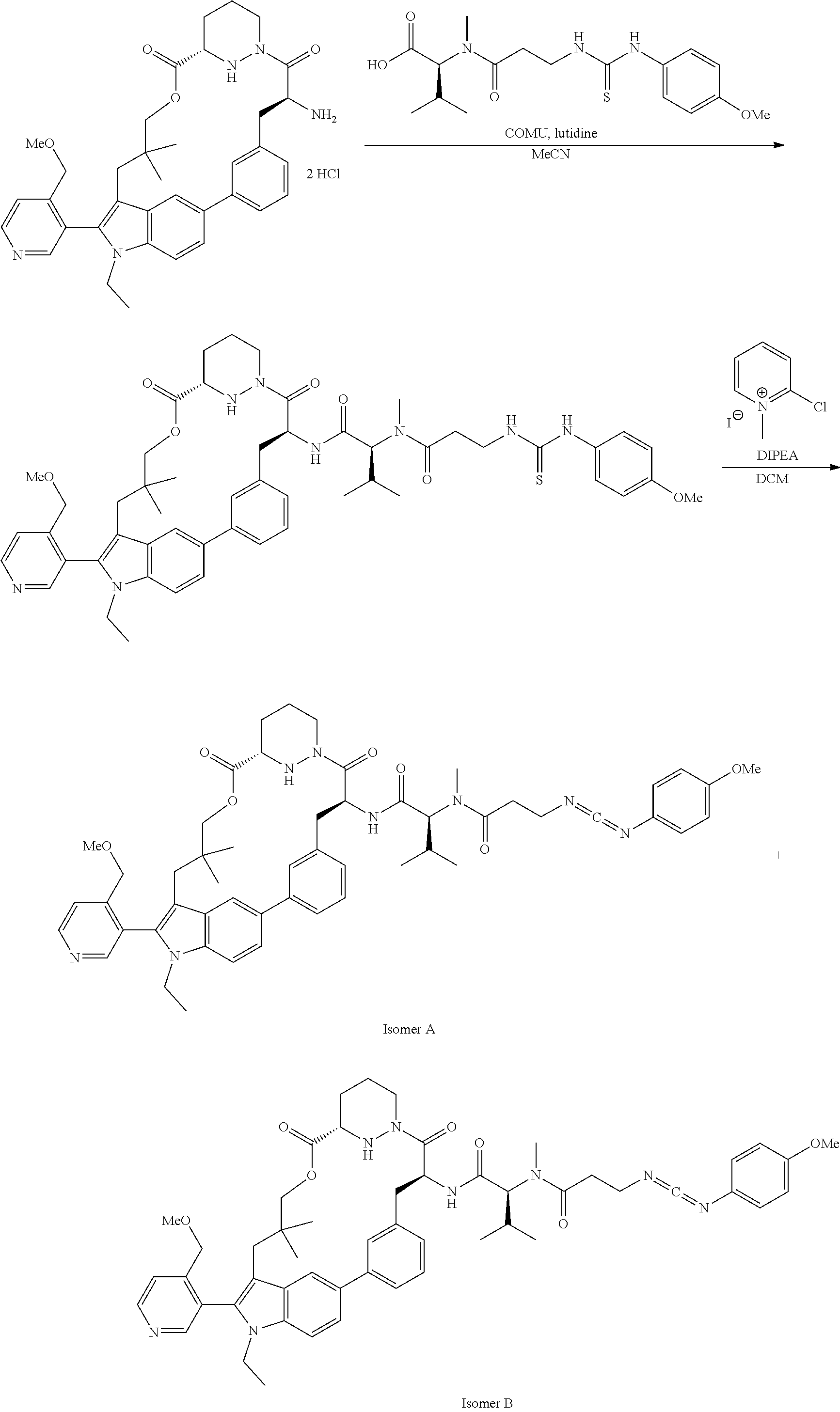

### Step 1: Synthesis of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(3-(3-(4-methoxyphenyl) thioureido)-N-methylpropanamido)-3-methylbutanamide

To a solution of (6³S,4S)-4-amino-1¹-ethyl-1²-(4-(methoxymethyl)pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (108 mg, 168 μmol) and N-(3-(3-(4-methoxyphenyl) thioureido) propanoyl)-N-methyl-L-valine (61.9 mg, 168 μmol) in MeCN (2 mL) at 0° C. was added 2,6-lutidine (97.8 μL, 840 μmol) followed by COMU (78.8 mg, 184 μmol). After 1 h at 0° C. the reaction was diluted with EtOAc and the organic portion washed with H₂O (15 mL) and brine (15 mL), dried over Na₂SO₄, and concentrated under reduced pressure. Purification by silica gel chromatography (20→100% EtOAc/Hex then 0→5% MeOH/EtOAc) afforded the desired product (117.0 mg 72.6% yield). LCMS (ESI) m/z: [M+H] calcd for C₅₃H₆₆N₈₀₇S: 959.49; found 959.5.

### Step 2: Synthesis of two isomers of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(3-((((4-methoxyphenyl) imino) methylene) amino)-N-methylpropanamido)-3-methylbutanamide

To a solution of (2S)—N-((6³S,4S)-1¹-ethyl-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(3-(3-(4-methoxyphenyl) thioureido)-N-methylpropanamido)-3-methylbutanamide (117.0 mg, 121 μmol) in DCM (1 mL) was added DIPEA (63.2 L, 363 μmol) followed by 2-chloro-1-methylpyridin-1-ium iodide (42.6 mg, 181 μmol). The reaction mixture was stirred overnight, at which point the solid was filtered and the crude solution was purified by reverse phase chromatography (40→100 MeCN/H₂O+0.4% NH₄OH) to afford two separated isomers as the desired earlier eluting isomer 15A (6.9 mg, 6.2% yield) and later eluting isomer 15B (2.5 mg, 2.2% yield). LCMS (ESI) m/z: [M+H] calcd for C₅₃H₆₄N₈O₇: 925.50; found 925.5 and LCMS (ESI) m/z: [M+H] calcd for C₅₃H₆₄N₈O₇: 925.50; found 925.6.

### Example 25. Synthesis of (2S)-2-(3-(3-(2-chloroethyl) ureido)-N-methylpropanamido)-N-((6³S,4S)-1¹-ethyl-25-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

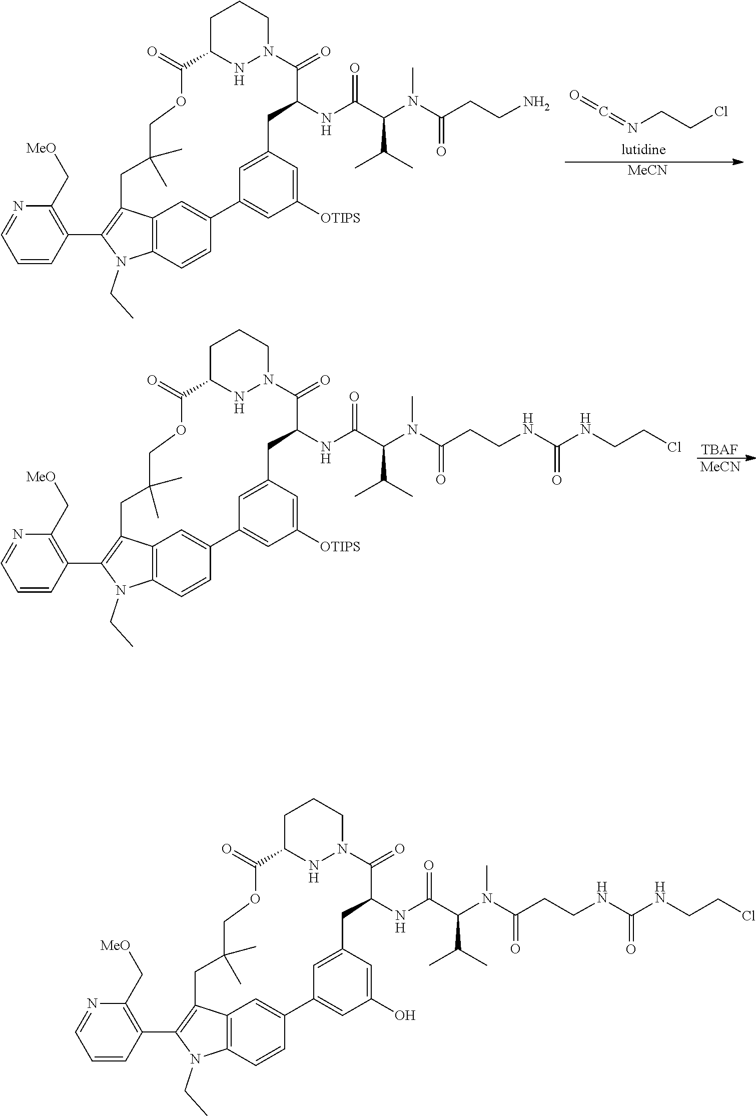

### Step 1: Synthesis of (2S)-2-(3-(3-(2-chloroethyl) ureido)-N-methylpropanamido)-N-((6³S,4S)-1¹-ethyl-1²-(2-(methoxymethyl)pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)-2-(3-amino-N-methylpropanamido)-N-((6³S,4S)-1¹-ethyl-1²-(2-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (106 mg, 109 μmol) in MeCN (544 μL) at 0° C. was added 1-chloro-2-isocyanatoethane (9.29 μL, 109 μmol) followed by Et₃N (15.1 μL, 109 μmol). After 12 min, the reaction was diluted with DCM (10 mL) and a solution of 1% formic acid in H₂O (10 mL). The aqueous layer was extracted with DCM (10 mL) and the combined organic layers were dried over Na₂SO₄, filtered, and then concentrated under reduced pressure to afford the desired product (117 mg, 100% yield), which was used in the next step without purification. LCMS (ESI) m/z: [M+H] calcd for C₅₇H₈₃ClN₈O₈Si: 1071.59; found 1071.5.

### Step 2: Synthesis of (2S)-2-(3-(3-(2-chloroethyl) ureido)-N-methylpropanamido)-N-((6³S,4S)-1¹-ethyl-25-hydroxy-1²-(2-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)-2-(3-(3-(2-chloroethyl) ureido)-N-methylpropanamido)-N-((6³S,4S)-1¹-ethyl-1²-(2-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (117 mg, 109 μmol) in MeCN (1.1 mL) at 0° C. was added TBAF (1M in dioxane, 109 μL, 109 μmol). After 5 min, the reaction was concentrated under reduced pressure and the crude residue was purified by normal phase chromatography (20→100% B/A, B=10% MeOH/EtOAc, A=hexanes) followed by reverse phase chromatography (20→60% MeCN/H₂O) to afford the final product (82.2 mg, 82% yield). LCMS (ESI) m/z: [M+H] calcd for C₄₈H₆₃ClN₈O₈: 915.45; found 915.7.

### Example 30. Synthesis of (2S)-2-(3-((4,5-dihydrooxazol-2-yl) amino)-N-methylpropanamido)-N-((6³S,4S)-1¹-ethyl-25-hydroxy-1²-(2-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

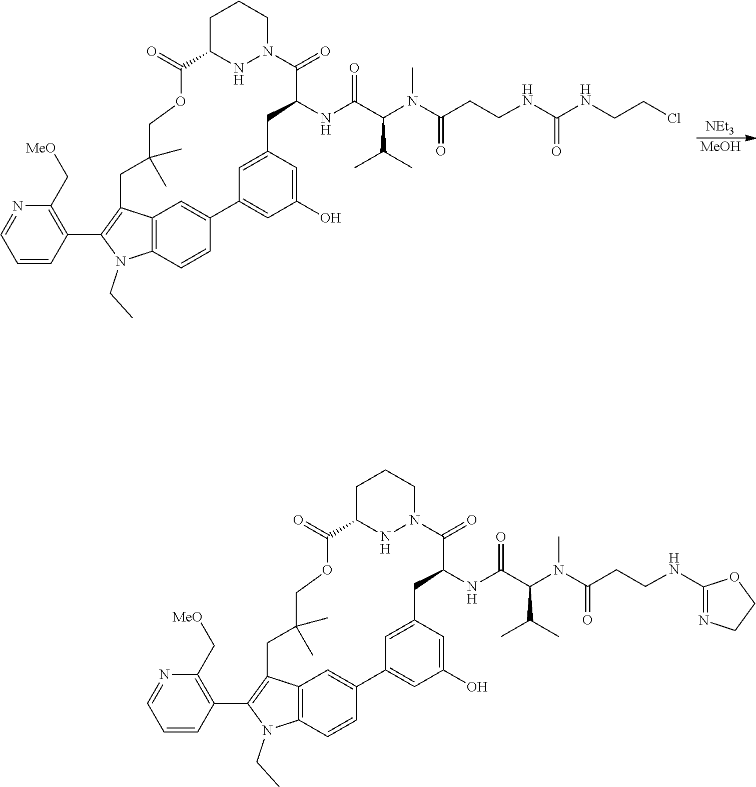 [SMILES: "MeOH" ; CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2 ; CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2 ; "NEt3"]

A solution of (2S)-2-(3-(3-(2-chloroethyl) ureido)-N-methylpropanamido)-N-((6³S,4S)-1¹-ethyl-25-hydroxy-1²-(2-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (55.0 mg, 60.0 μmol) and Et₃N (25.1 μL, 180 μmol) in MeOH (1.2 mL) was heated in the microwave at 150° C. for 1 min. The reaction mixture was cooled to room temperature and concentrated under reduced pressure. The crude residue was then purified by reverse phase chromatography (30→100% MeCN/H₂O+0.4% NH₄OH) to afford the final product (21.1 mg, 40% yield). LCMS (ESI) m/z: [M+H] calcd for C₄₈H₆₂N₈O₈: 879.48; found 879.4.

### Example 31. Synthesis of (3S)—N-((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-1-((S)-oxirane-2-carbonyl) pyrrolidine-3-carboxamide

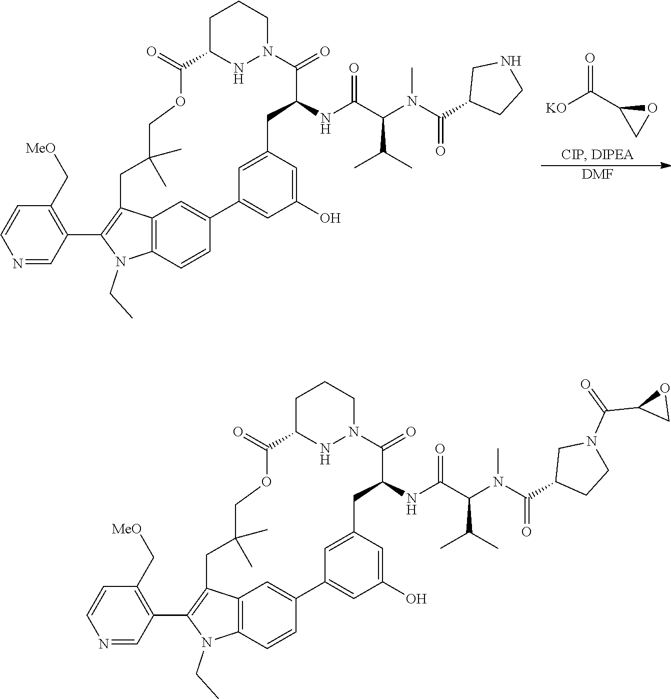

To a solution of potassium(S)-oxirane-2-carboxylate (16.98 mg, 0.135 mmol), 2-chloro-1,3-dimethylimidazolidinium hexafluorophosphate (87.46 mg, 0.314 mmol), and DIPEA (0.156 mL, 0.897 mmol) in DMF (1.5 mL) at 0° C. was added (3S)—N-((2S)-1-(((6³S,4S)-1¹-ethyl-25-hydroxy-12. (4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpyrrolidine-3-carboxamide (75.0 mg, 0.09 mmol). The resulting mixture was stirred overnight at room temperature, at which point it was diluted with EtOAc (100 mL). The organic layer was washed with brine (3×5 mL), dried over Na₂SO₄, filtered, and concentrated under reduced pressure. Purification by reverse phase chromatography (25→55% MeCN/H₂O) afforded the desired product (6.3 mg, 7.8% yield) as a solid. LCMS (ESI) m/z: [M+H] calcd for C₅₀H₆₃N₇O₉: 906.48; found 906.7.

### Example 34. Synthesis of (2R)-1-acetyl-N-(2-(((2S)-1-(((6³S,4S)-1¹-ethyl-25-hydroxy-12-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

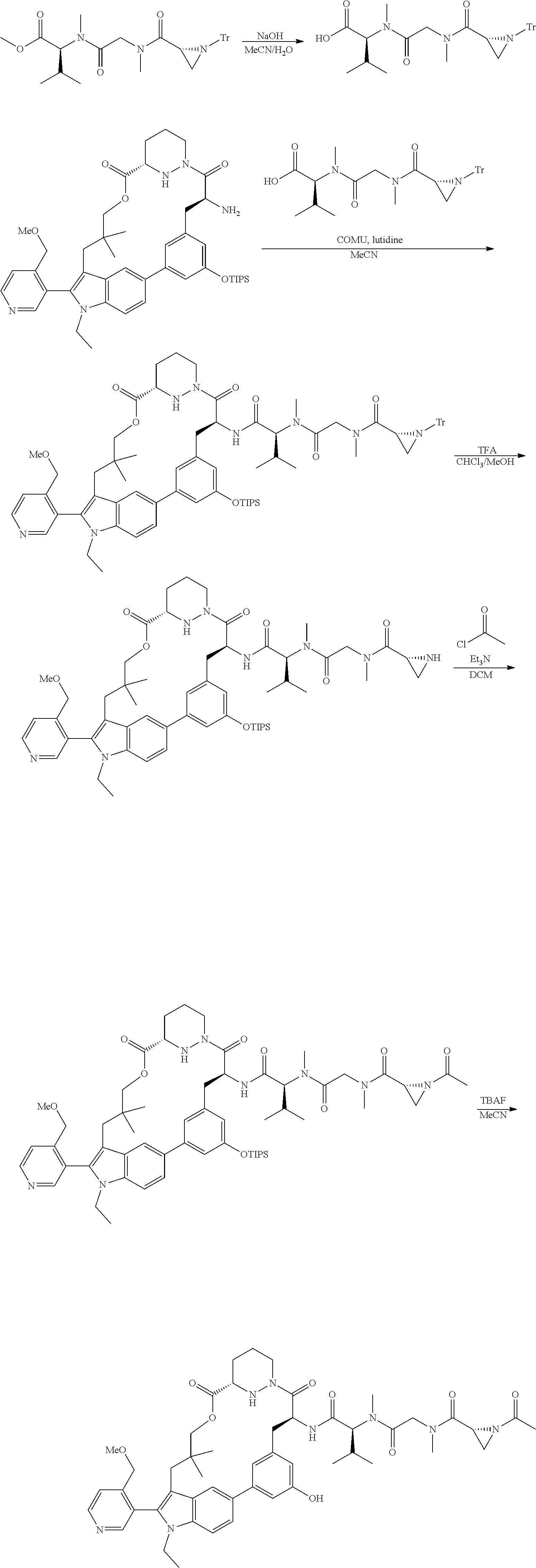

### Step 1: Synthesis of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide

To a solution of (6³S,4S)-4-amino-1¹-ethyl-1²-(4-(methoxymethyl)pyridin-3-yl)-10,10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (260 mg, 0.332 mmol) and N-methyl-N—(N-methyl-N—((R)-1-tritylaziridine-2-carbonyl) glycyl)-L-valine (204 mg, 0.399 mmol) in MeCN (3.3 mL) at 0° C. was added lutidine (192 μL, 1.66 mmol) followed by COMU (156 mg, 0.366 mmol). The reaction stirred at 0° C. for 1 h and was then diluted with EtOAc. The mixture was washed with H₂O/brine (1:1), dried over Na₂SO₄, filtered, and concentrated under reduced pressure. Purification by normal phase chromatography (0→100% EtOAc/hexanes) afforded the desired product (116 mg, 27% yield). LCMS (ESI) m/z: [M+H] calcd for C₇₆H₉₆N₈O₈Si: 1277.72; found 1277.7.

### Step 2: Synthesis of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide (400 mg, 0.313 mmol) in MeOH (1.56 mL) and chloroform (1.56 mL) at 0° C. was added TFA (191 μL, 2.50 mmol). The reaction stirred at 0° C. for 2 h and was then quenched with lutidine (364 μL, 3.13 mmol). The reaction mixture was diluted with DCM, washed with H₂O, and concentrated under reduced pressure. Purification by reverse phase chromatography (10→100% MeCN/H₂O) afforded the desired product (100 mg, 31% yield). LCMS (ESI) m/z: [M+H] calcd for C₅₇H₈₂N₈O₈Si: 1035.61; found 1035.6.

### Step 3: Synthesis of (2R)-1-acetyl-N-(2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (33 mg, 0.032 mmol) in DCM (637 μL) at 0° C. was added Et₃N (22.1 μL, 0.159 mmol) followed by acetyl chloride (4.54 μL, 0.064 mmol). The reaction stirred at 0° C. for 1 h. The reaction was then diluted with DCM, washed with NaHCO₃, dried over Na₂SO₄, filtered, and concentrated under reduced pressure to afford the desired crude product (37 mg, 100% yield). LCMS (ESI) m/z: [M+H] calcd for C₅₉H₈₄N₈O₉Si: 1077.62; found 1077.6.

### Step 4: Synthesis of (2R)-1-acetyl-N-(2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2R)-1-acetyl-N-(2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (34 mg, 0.032 mmol) in MeCN (631 μL) at 0° C. was added TBAF (1M in THF, 31.5 μL, 0.032 mmol). The reaction stirred for 10 min and was then diluted with DCM, washed with brine, dried over Na₂SO₄, filtered, and concentrated under reduced pressure. Purification by reverse phase chromatography (10→100% MeCN/H₂O) afforded the desired product (8.5 mg, 29% yield). LCMS (ESI) m/z: [M+H] calcd for C₅₀H₆₄N₈O₉: 921.49; found 921.5.

### Example 36. Synthesis of (2R)—N-(2-(((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(methylsulfonyl) aziridine-2-carboxamide

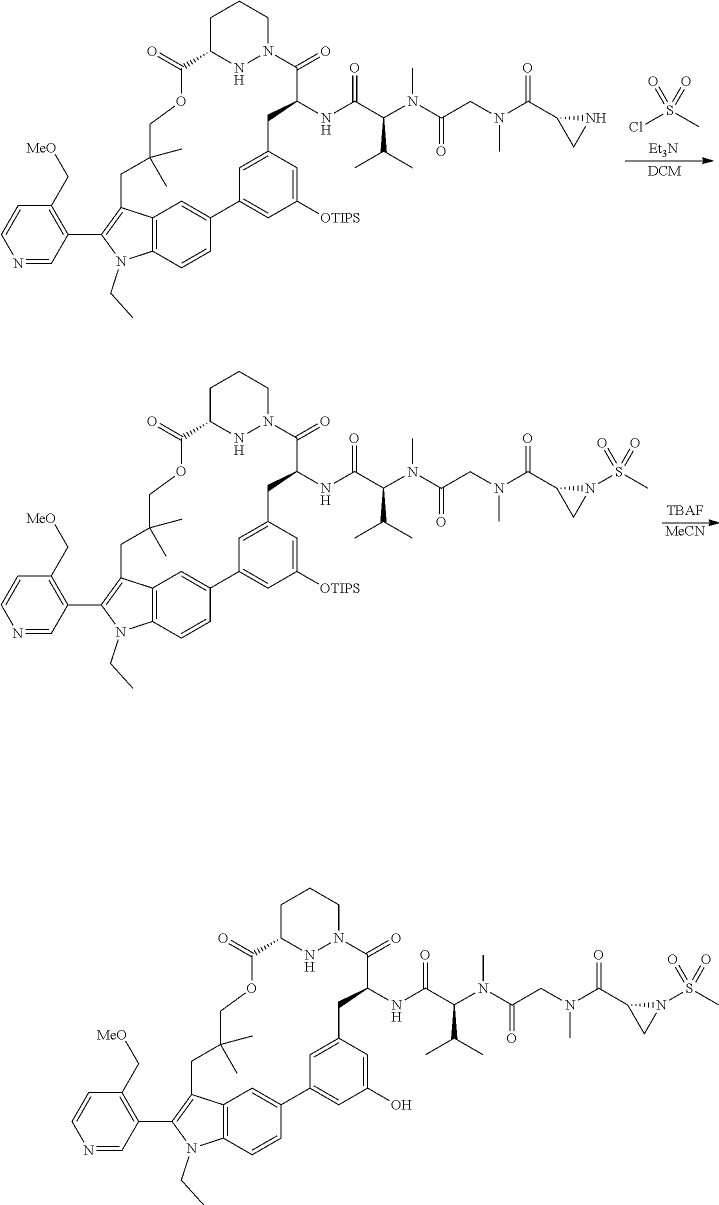

### Step 1: Synthesis of (2R)—N-(2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(methylsulfonyl) aziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (33 mg, 0.032 mmol) in DCM (637 μL) at 0° C. was added Et₃N (22.1 μL, 0.159 mmol) followed by methanesulfonyl chloride (4.93 UL, 0.064 mmol). The reaction was cooled to 0° C. for 1 h and was then diluted with DCM, washed with NaHCO₃, dried over Na₂SO₄, filtered, and concentrated under reduced pressure to afford the desired crude product (35 mg, 100% yield). LCMS (ESI) m/z: [M+H] calcd for C₅₈H₈₄N₈O₁₀SSi: 1113.59; found 1113.6.

### Step 2: Synthesis of (2R)—N-(2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(methylsulfonyl) aziridine-2-carboxamide

To a solution of (2R)—N-(2-(((2S)-1-(((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(methylsulfonyl) aziridine-2-carboxamide (35 mg, 0.032 mmol) in MeCN (646 μL) at 0° C. was added TBAF (1M in THF, 32.3 μL, 0.032 mmol). The reaction stirred for 10 min and was then diluted with DCM, washed with brine, dried over Na₂SO₄, filtered, and concentrated under reduced pressure. Purification by reverse phase chromatography (10→100% MeCN/H₂O) afforded the desired product (20 mg, 65% yield). LCMS (ESI) m/z: [M+H] calcd for C₄₉H₆₄N₈O₁₀S: 957.45; found 957.5.

### Example 38. Synthesis of methyl (2R)-2-((2-(((2S)-1-(((6³S,4S)-1¹-ethyl-25-hydroxy-12-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-1-carboxylate

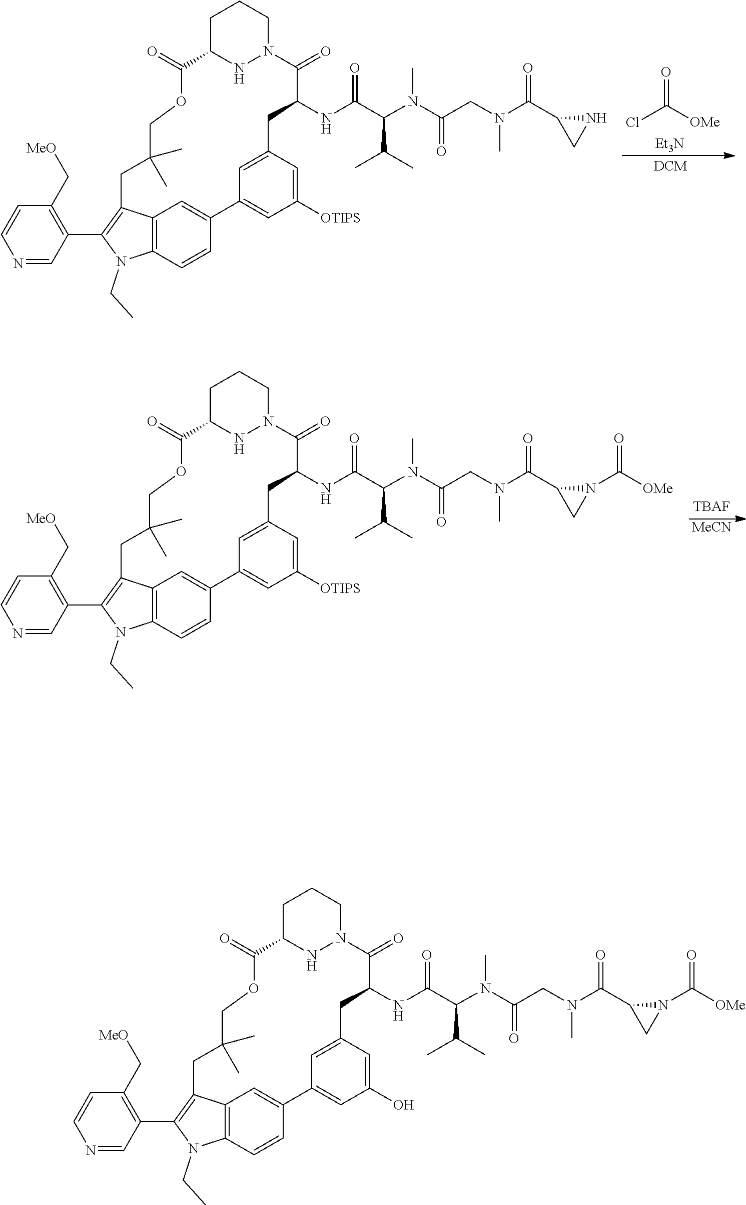

### Step 1: Synthesis of methyl (2R)-2-((2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-1-carboxylate

To a solution of (2R)—N-(2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (46 mg, 0.044 mmol) in DCM (888 μL) at 0° C. was added E3N (30.8 μL, 0.22 mmol) followed by methyl chloroformate (4.46 μL, 0.058 mmol). The reaction stirred at 0° C. for 1 h and then the reaction was diluted with DCM, washed with NaHCO₃, dried over Na₂SO₄, filtered, and concentrated under reduced pressure to afford the desired crude product (56 mg, 100% yield). LCMS (ESI) m/z: [M+H] calcd for C₅₉H₈₄N₈O₁₀Si: 1093.62; found 1093.7.

### Step 2: Synthesis of methyl (2R)-2-((2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-1-carboxylate

To a solution of methyl (2R)-2-((2-(((2S)-1-(((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-1-carboxylate (56 mg, 0.051 mmol) in MeCN (1.0 mL) at 0° C. was added TBAF (1M in THF, 51.2 μL, 0.051 mmol). The reaction stirred for 15 min and was then diluted with DCM, washed with brine, dried over Na₂SO₄, filtered, and concentrated under reduced pressure. Purification by reverse phase chromatography (10→100% MeCN/H₂O) afforded the desired product (17 mg, 36% yield). LCMS (ESI) m/z: [M+H] calcd for C₅₀H₆₄N₈O₁₀: 937.48; found 937.6.

### Example 48 and 49. Synthesis of methyl (2S,3R)-1-((R)-tert-butylsulfinyl)-3-((2-(((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-2-carboxylate and methyl (2S,3R)-3-((2-(((2S)-1-(((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-2-carboxylate

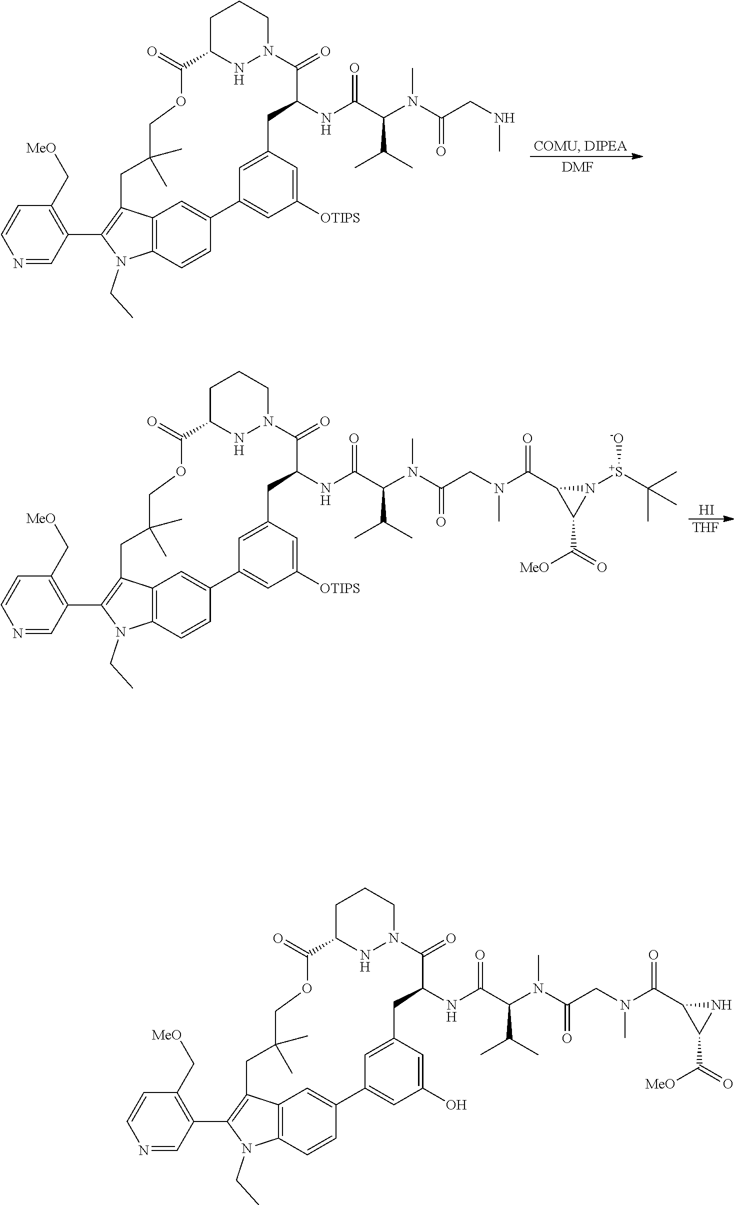

### Step 1: Synthesis of methyl (2S,3R)-1-((R)-tert-butylsulfinyl)-3-((2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-2-carboxylate

To a solution of (2S)—N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide (267.0 mg, 0.33 mmol) and (2R,3S)-1-((R)-tert-butylsulfinyl)-3-(methoxycarbonyl) aziridine-2-carboxylic acid (246.5 mg, 0.99 mmol) in DMF (4.5 mL) at 0° C. was added DIPEA (0.574 mL, 3.3 mmol) followed by a solution of COMU (211.8 mg, 0.49 mmol) in DMF (0.5 mL). The resulting mixture was stirred for 1 h at 0° C. and was then quenched with sat. NH₄Cl. The aqueous layer was extracted with EtOAc (3×20 mL) and the combined organic layers were washed with brine (2×50 mL), dried over Na₂SO₄, filtered, concentrated under reduced pressure. The crude product was purified by reverse phase chromatography (35→65% MeCN/H₂O) to afford the desired product (253 mg, 73.7% yield). LCMS (ESI) m/z: [M+H] calcd for C₅₄H₇₂N₈O₁₁S: 1041.51; found 1041.8.

### Step 2: Synthesis of methyl (2S,3R)-3-((2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-2-carboxylate

To a solution of methyl (2S,3R)-1-((R)-tert-butylsulfinyl)-3-((2-(((2S)-1-(((6³S,4S)-1¹-ethyl-25-hydroxy-1²-(4-(methoxymethyl)pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl) (methyl) carbamoyl) aziridine-2-carboxylate (200.0 mg, 0.19 mmol) in THF (4.0 mL) at 0° C. was added HI (1.0 mL), dropwise. The resulting mixture was stirred for 10 min at 0° C. and was then basified to pH 7 with DIPEA. The mixture was extracted with EtOAc (3×30 mL) and the combined organic layers were washed with brine (50 mL), dried over Na₂SO₄, filtered, and concentrated under reduced pressure. The residue was purified by reverse phase chromatography (35→65% MeCN/H₂O) to afford the desired product (13.2 mg, 7.3% yield). LCMS (ESI) m/z: [M+H] calcd for C₅₀H₆₄N₈O₁₀: 937.48; found 938.6.

### Example 55. Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

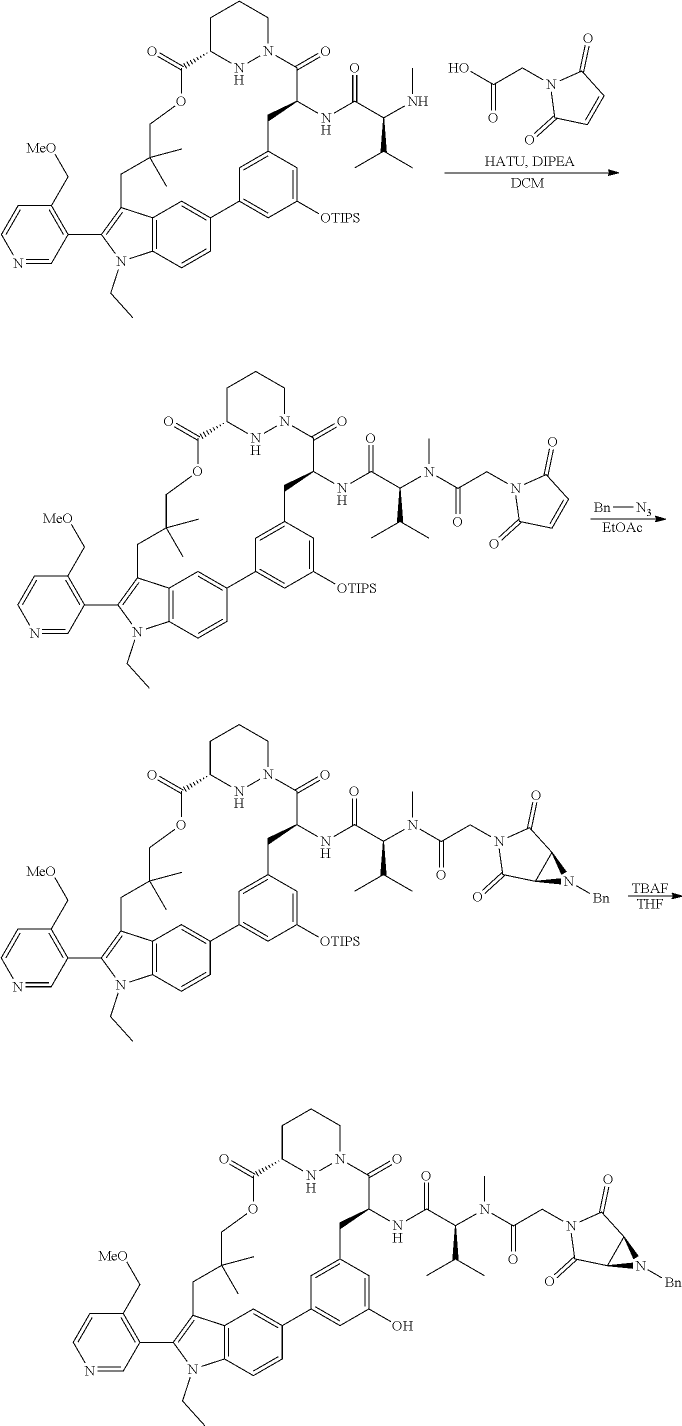

### Step 1: Synthesis of (2S)-2-(2-(2,5-dioxo-2,5-dihydro-1H-pyrrol-1-yl)-N-methylacetamido)-N-((6³S,4S)-1¹-ethyl-1²-(4-(methoxymethyl)pyridin-3-yl)-10, 10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)—N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (600.0 mg, 0.67 mmol) and 2-(2,5-dioxo-2,5-dihydro-1H-pyrrol-1-yl) acetic acid (124.7 mg, 0.80 mmol) in DCM (6.0 mL) at 0° C. was added DIPEA (0.934 mL, 5.36 mmol) followed by HATU (382.2 mg, 1.01 mmol). The reaction mixture was warmed to room temperature and stirred for 3 h. The reaction was then quenched by the addition of H₂O (20 mL). The aqueous layer was extracted with DCM (2×50 mL) and the combined organic layers were washed with brine (2×50 mL), dried over Na₂SO₄, and concentrated under reduced pressure. The residue was purified by normal phase chromatography (10→20% EtOAc/pet. ether) to afford the desired product (260 mg, 33.8% yield). LCMS (ESI) m/z: [M+H] calcd for C₅₇H₇₇N₇O₉Si: 1032.56; found 1032.8.

### Step 2: Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-1²-(4-(methoxymethyl)pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)-2-(2-(2,5-dioxo-2,5-dihydro-1H-pyrrol-1-yl)-N-methylacetamido)-N-((6³S,4S)-1¹-ethyl-1²-(4-(methoxymethyl)pyridin-3-yl)-10, 10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (250.0 mg, 0.24 mmol) in EtOAc (2.0 mL) was added (azidomethyl)benzene (80.6 mg, 0.61 mmol). The reaction mixture was heated to 80° C. and stirred for 2 h. The reaction mixture was then heated to 120° C. and stirred for 2 days. The reaction mixture was then cooled to room temperature and quenched with H₂O. The aqueous layer was extracted with EtOAc and the combined organic layers were washed with brine, dried with Na₂SO₄, filtered, and concentrated under reduced pressure. The residue was purified by normal phase chromatography to afford the desired product (50 mg, 18.1% yield). LCMS (ESI) m/z: [M+H] calcd for C₆₄H₈₄N₈O₉Si: 1137.62; found 1138.3.

### Step 3: Synthesis of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-25-hydroxy-1²-(4-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)-2-(2-((1R,5S)-6-benzyl-2,4-dioxo-3,6-diazabicyclo[3.1.0]hexan-3-yl)-N-methylacetamido)-N-((6³S,4S)-1¹-ethyl-1²-(4-(methoxymethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide (50.0 mg, 0.04 mmol) in THF (0.5 mL) at 0° C. was added 1M TBAF (0.07 mL, 0.07 mmol). The reaction mixture was stirred for 1 h. The reaction mixture was then diluted with H₂O and extracted with EtOAc. The combined organic layers were washed with brine, dried over Na₂SO₄, filtered, and concentrated under reduced pressure. The residue was purified by prep-TLC followed by reverse phase chromatography (45→72% MeCN/H₂O) to afford the desired product (20 mg, 46.4% yield). LCMS (ESI) m/z: [M+Na] calcd for C₅₅H₆₄N₈O₉: 1003.47; found 1003.8.

### Example 95. Synthesis of (2R)—N-(2-(((1S)-1-cyclopentyl-2-(((6³S,4S)-1¹-ethyl-25-hydroxy-1²-(2-((S)-1-methoxyethyl) pyridin-3-yl)-10,10-dimethyl-5,7-dioxo-61,62,63,64,65,66-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methylaziridine-2-carboxamide

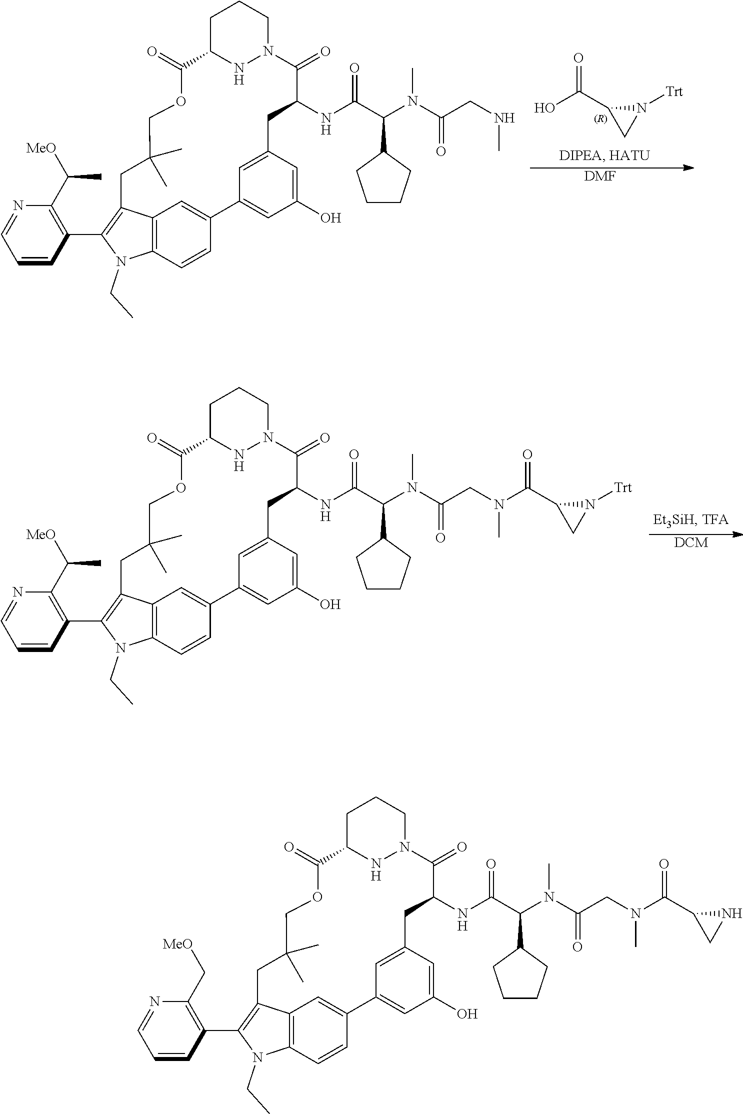

### Step 1: Synthesis of (2R)—N-(2-(((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methyl-1-tritylaziridine-2-carboxamide

To a mixture of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(N-methyl-2-(methylamino) acetamido) acetamide (321.2 mg, 0.276 mmol), DIPEA (0.472 mL, 2.764 mmol), and (R)-1-tritylaziridine-2-carboxylic acid (136.59 mg, 0.415 mmol) in DMF (3.0 mL) at 0° C. was added HATU (126.14 mg, 0.332 mmol). The resulting mixture was stirred at 0° C. for 30 min, then diluted with H₂O (30 mL) and extracted with EtOAc (3×30 mL). The combined organic layers were washed with brine (3×10 mL), dried with Na₂SO₄, filtered, and concentrated under reduced pressure. Purification by prep-TLC(50% EtOAc/pet. ether) afforded the desired product (200 mg, 62.3% yield). LCMS (ESI) m/z: [M+H] calcd for C₇₀H₈₀N₈O₈: 1161.62; found 1161.5.

### Step 2: Synthesis of (2R)—N-(2-(((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methylaziridine-2-carboxamide

To a mixture of (2R)—N-(2-(((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methyl-1-tritylaziridine-2-carboxamide (195.0 mg, 0.168 mmol) in DCM (2.0 mL) at 0° C. was added Et₃SiH (78.09 mg, 0.672 mmol) and TFA (76.57 mg, 0.672 mmol). The resulting mixture was stirred at 0° C. for 30 min then basified to pH 8 with DIPEA and concentrated under reduced pressure. Purification by reverse phase chromatography (25→55% MeCN/H₂O) to afford the desired product (60 mg, 38.9% yield). LCMS (ESI) m/z: [M+H] calcd for C₅₁H₆₆N₈O₈: 919.51; found 919.5.

### Example 87. Synthesis of 6-((S)-aziridin-2-yl)-N-((2S)-1-(((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-11H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide

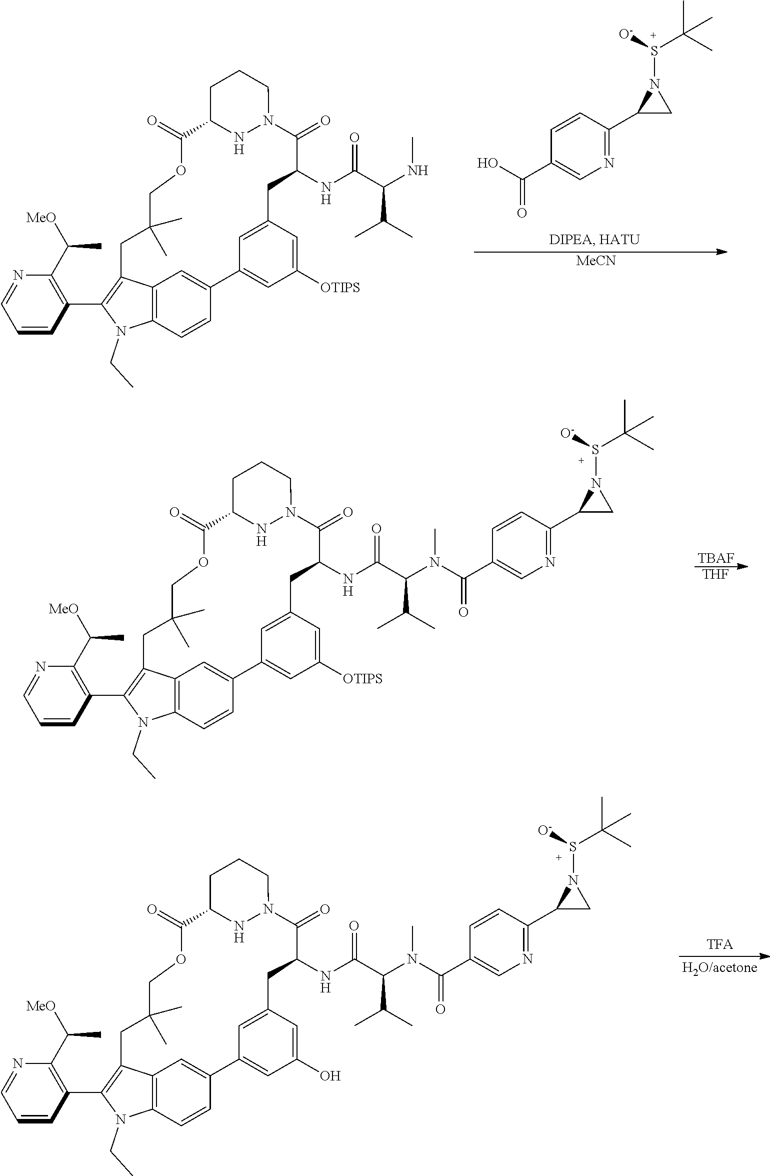

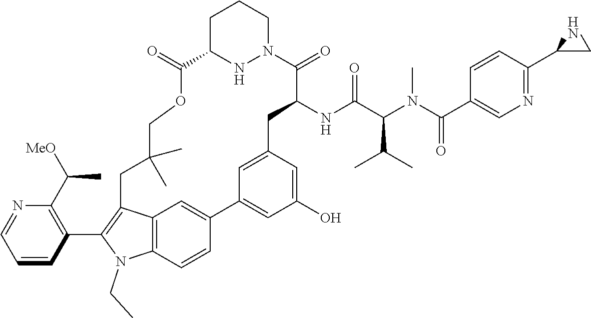

### Step 1: Synthesis of 6-((2S)-1-(tert-butylsulfinyl) aziridin-2-yl)-N-((2S)-1-(((6³S,4S)-1¹-ethyl-12-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide

To a mixture of (2S)—N-((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(methylamino) butanamide (198.24 mg, 0.218 mmol) and DIPEA (0.074 mL, 0.436 mmol) in MeCN (10 mL) at 0° C. was added HATU (200 mg, 0.526 mmol) and the resulting mixture was stirred for 3 min. To the mixture was then added a solution of 6-((2S)-1-(tert-butylsulfinyl) aziridin-2-yl) nicotinic acid (117.0 mg, 0.436 mmol) in MeCN (10 mL) in portions. The resulting mixture was stirred overnight at 0° C. and then was then quenched with H₂O extracted with EtOAc (3×20 mL). The combined organic layers were washed with brine (20 mL), dried with Na₂SO₄, filtered, and concentrated under reduced pressure to afford the desired product (430 mg, 85.0% yield). LCMS (ESI) m/z: [M+H] calcd for C₆₄H₉₀N₈O₈SSi: 1159.65; found 1159.8.

### Step 2: Synthesis of 6-((2S)-1-(tert-butylsulfinyl) aziridin-2-yl)-N-((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide

To a solution of 6-((2S)-1-(tert-butylsulfinyl) aziridin-2-yl)-N-((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide (430.0 mg, 0.371 mmol) in THF (50.0 mL) at 0° C. was added TBAF (1 M in THF, 1.1 mL, 1.11 mmol) in portions. The resulting mixture was stirred at 0° C. for 2 h and was then concentrated under reduced pressure. The residue was purified by prep-TLC(5% MeOH/DCM) to afford the desired product (290 mg, 78% yield). LCMS (ESI) m/z: [M+H] calcd for C₅₅H₇₀N₈O₈S₈ 1003.51; found 1003.8.

### Step 3: Synthesis of 6-((S)-aziridin-2-yl)-N-((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide

To a solution of 6-((2S)-1-(tert-butylsulfinyl) aziridin-2-yl)-N-((2S)-1-(((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylnicotinamide (150.0 mg, 0.150 mmol) in H₂O (15.0 mL) and acetone (15.0 mL) at 0° C. was added TFA (7.50 mL, 100.97 mmol) in portions. The resulting mixture was warmed to room temperature and stirred for 48 h and then was neutralized to pH 8 with sat. NaHCO₃. The aqueous layer was extracted with EtOAc, dried with Na₂SO₄, filtered, and concentrated under reduced pressure. Purification by reverse phase chromatography (38→58% MeCN/H₂O) afforded the desired product (10.0 mg, 7.4% yield). LCMS (ESI) m/z: [M+H] calcd for C₅₁H₆₂N₈O₇: 899.48; found 899.5.

### Example 139. Synthesis of (2S)-2-((S)-7-(((R)-aziridin-2-yl)methyl)-1-oxo-2,7-diazaspiro[4.4]nonan-2-yl)-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

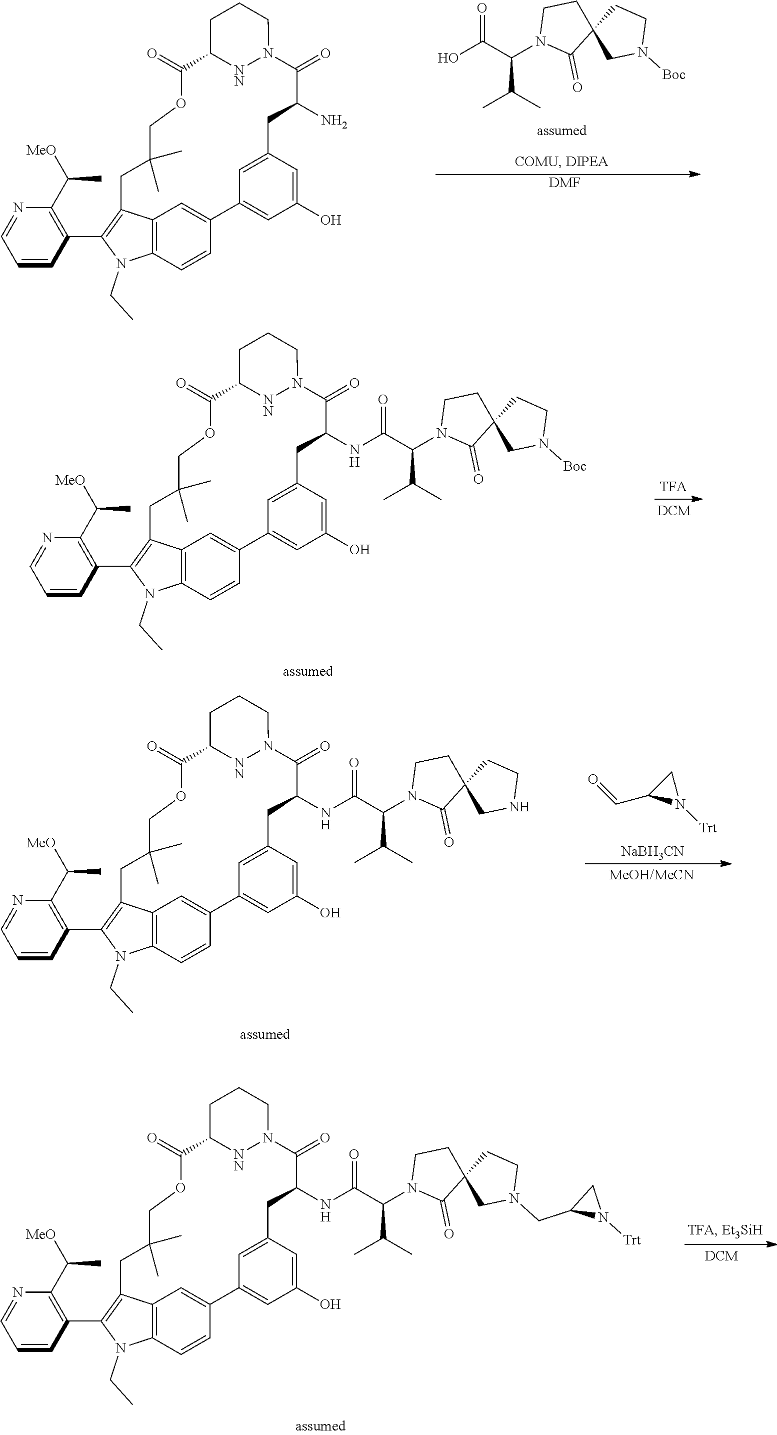

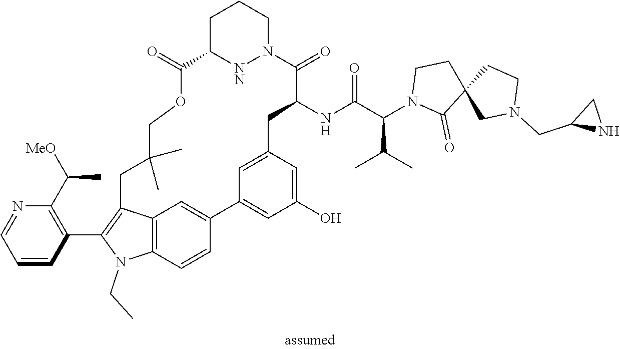

### Step 1: Synthesis of tert-butyl (5R)-7-((2S)-1-(((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-6-oxo-2,7-diazaspiro[4.4]nonane-2-carboxylate

To a solution of (6³S,4S)-4-amino-1¹-ethyl-25-hydroxy-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (600 mg, 0.94 mmol) and DIPEA (820 μL, 4.7 mmol) in DMF (8 mL) at 0° C. was added(S)-2-((R)-7-(tert-butoxycarbonyl)-1-oxo-2,7-diazaspiro[4.4]nonan-2-yl)-3-methylbutanoic acid (380 mg, 1.13 mmol) and COMU (440 mg, 1.03 mmol). The reaction mixture was stirred for 1 h then was diluted with H₂O (100 mL) and extracted with EtOAc (3×50 mL). The combined organic layers were washed with brine (30 mL), dried with Na₂SO₄, filtered, and the filtrate was concentrated under reduced pressure. Purification by Prep-TLC(EtOAc) afforded the desired product (600 mg, 66% yield). LCMS (ESI) m/z: [M+H] calcd for C₅₄H₇₁N₇O₉: 962.54; found 962.5.

### Step 2: Synthesis of (2S)—N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-2,7-diazaspiro[4.4]nonan-2-yl) butanamide

To a solution of tert-butyl (5R)-7-((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-6-oxo-2,7-diazaspiro[4.4]nonane-2-carboxylate (600 mg, 0.62 mmol) in DCM (6 mL) at 0° C. was added TFA (3.0 mL, 40 mmol). The reaction mixture was stirred for 2 h and then was concentrated under reduced pressure. The residue was diluted with H₂O (100 mL), basified to pH 8 with sat. aq. NaHCO₃, and extracted with EtOAc (3×60 mL). The combined organic layers were washed with brine (30 mL), dried with Na₂SO₄, filtered, and concentrated under reduced pressure to afford the desired product (430 mg, 79% yield). LCMS (ESI) m/z: [M+H] calcd for C₄₉H₆₃N₇O₇: 862.49; found 862.5.

### Step 3: Synthesis of (2S)—N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-7-(((S)-1-tritylaziridin-2-yl)methyl)-2,7-diazaspiro [4.4]nonan-2-yl) butanamide

To a solution of (2S)—N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-2,7-diazaspiro[4.4]nonan-2-yl) butanamide (200 mg, 0.23 mmol) and (R)-1-tritylaziridine-2-carbaldehyde (110 mg, 0.35 mmol) in MeOH (0.50 mL) and MeCN (4.0 mL) was added NaBH₃CN (29 mg, 0.46 mmol). The reaction mixture was stirred for 2 h then was quenched with sat. aq. NH₄Cl and was extracted with EtOAc (3×50 mL). The combined organic layers were washed with brine (30 mL), dried with Na₂SO₄, filtered, and concentrated under reduced pressure. Purification by Prep-TLC(EtOAc) afforded desired product (145 mg, 53% yield). LCMS (ESI) m/z: [M+H] calcd for C₇₁H₈₂N₈O₇: 1159.64; found 1159.6.

### Step 4: Synthesis of (2S)-2-((S)-7-(((R)-aziridin-2-yl)methyl)-1-oxo-2,7-diazaspiro[4.4]nonan-2-yl)-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methylbutanamide

To a solution of (2S)—N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-((S)-1-oxo-7-(((S)-1-tritylaziridin-2-yl)methyl)-2,7-diazaspiro[4.4]nonan-2-yl) butanamide (140 mg, 0.12 mmol) in DCM (2.0 mL) 0° C. was added TFA (74 μL, 0.97 mmol) and Et₃SiH (150 μL, 0.97 mmol). The reaction mixture was stirred for 30 min then was basified to pH 8 with DIPEA. The resulting mixture was concentrated under reduced pressure. Purification by reverse phase chromatography (30→60% MeCN/H₂O) afforded the desired product (37.5 mg, 31% yield). LCMS (ESI) m/z: [M+H] calcd for C₅₂H₆₈N₈O₇: 917.53; found 917.4.

### Example 133. Synthesis of (2R,3R)-3-cyclopropyl-N-(2-(((2S)-1-(((6³S,4S)-1¹-ethyl-25-hydroxy-1²-(2-((S)-1-methoxyethyl) pyridin-3-yl)-10,10-dimethyl-5,7-dioxo-61,62,63,64,65,66-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

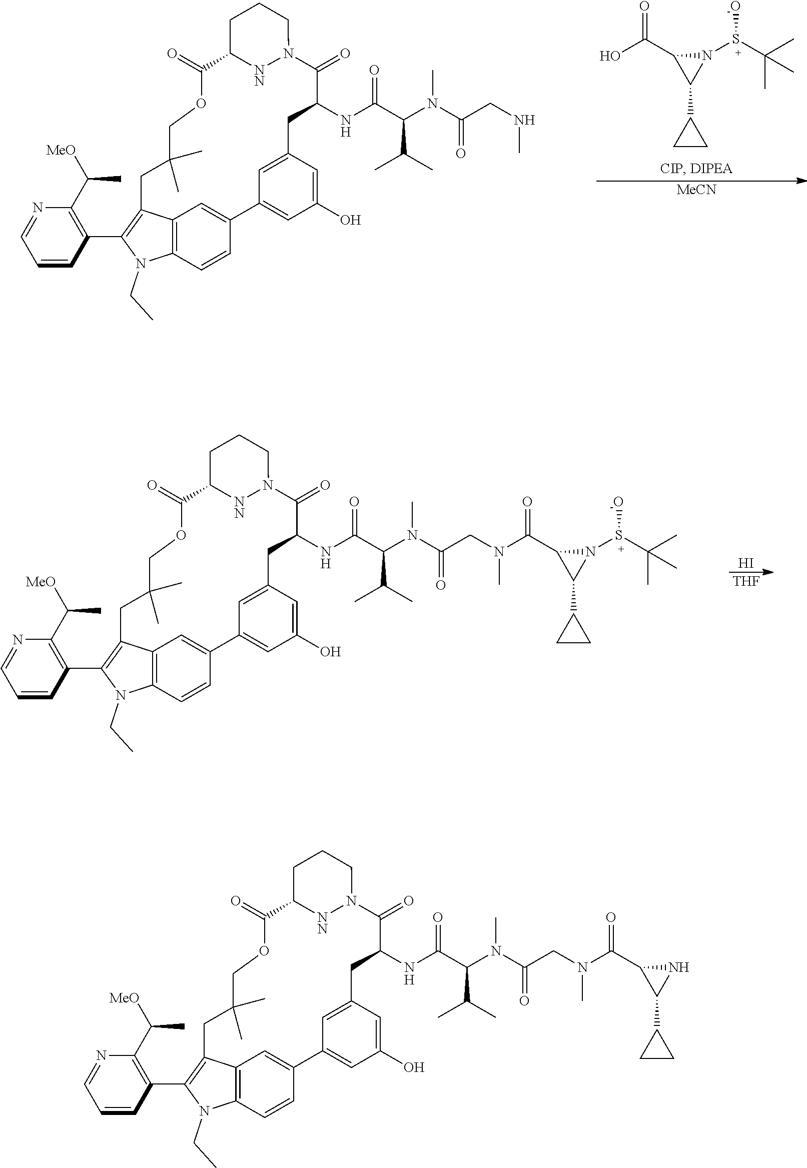

### Step 1: Synthesis of (2R,3R)-1-(tert-butylsulfinyl)-3-cyclopropyl-N-(2-(((2S)-1-(((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2S)—N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide (50 mg, 61 μmol) and (2R,3R)-1-(tert-butylsulfinyl)-3-cyclopropylaziridine-2-carboxylic acid (21 mg, 91 μmol) in MeCN at 0° C. was added DIPEA (210 UL, 1.2 mmol) and CIP (25 mg, 91 μmol). The resulting mixture was stirred for 2 h and was then concentrated under reduced pressure. Purification by Prep-TLC(9% EtOAc/pet. ether) afforded the desired product (270 mg, 54% yield). LCMS (ESI) m/z: [M+H] calcd for C₅₆H₇₆N₈O₉S: 1037.56; found 1037.4.

### Step 2: Synthesis of (2R,3R)-3-cyclopropyl-N-(2-(((2S)-1-(((6³S,4S)-1¹-ethyl-25-hydroxy-12-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a mixture of (2R,3R)-1-(tert-butylsulfinyl)-3-cyclopropyl-N-(2-(((2S)-1-(((6³S,4S)-1¹-ethyl-2⁵-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (230 mg, 0.22 mmol) in THF at 0° C. was added HI (0.50 mL, 3.8 mmol, 57% wt in H₂O). The reaction mixture was stirred for 10 min and then neutralized to pH 8 with DIPEA and concentrated under reduced pressure. Purification by reverse phase chromatography (40→60% MeCN/H₂O) afforded desired product (20 mg, 11% yield) as a white solid. LCMS (ESI) m/z: [M+H] calcd for C₅₂H₆₈N₈O₈: 933.53; found 933.6.

### Example 177. Synthesis of 4-((R)-aziridine-2-carbonyl)-N-((2S)-1-(((6³S,4S)-1¹-ethyl-2⁵-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperazine-1-carboxamide

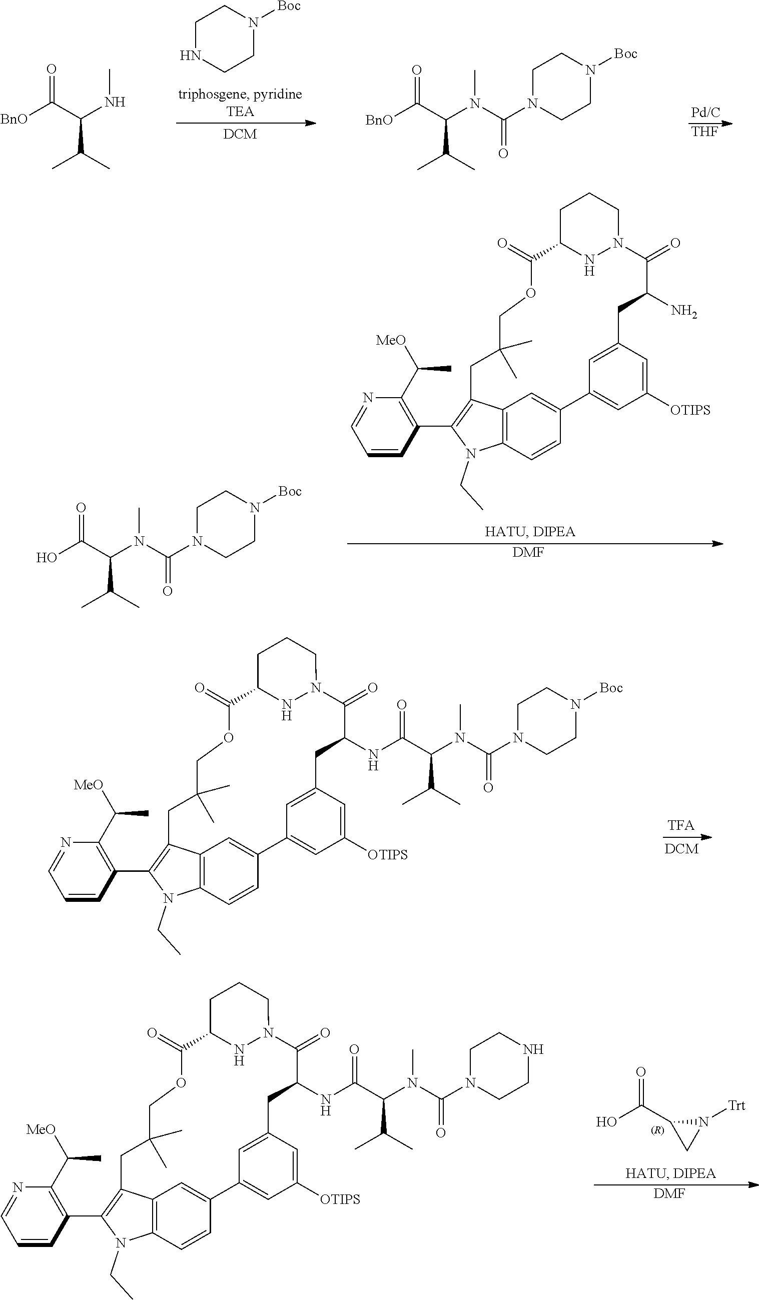

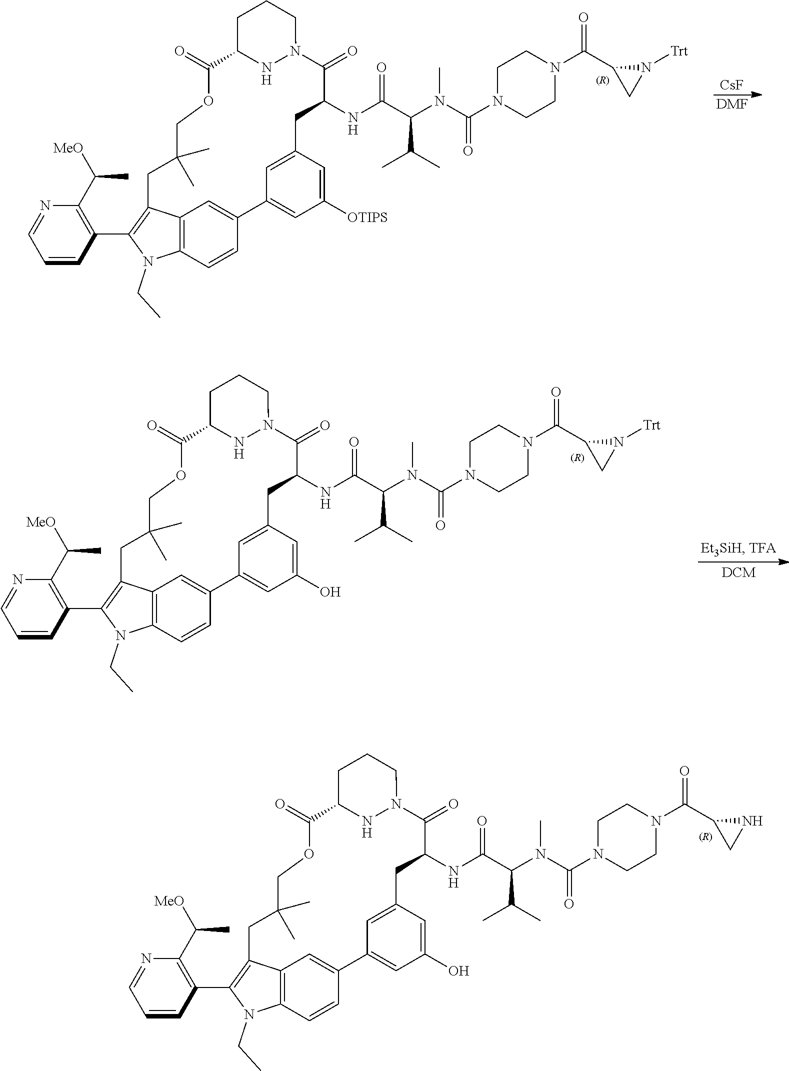

### Step 1: Synthesis of tert-butyl(S)-4-((1-(benzyloxy)-3-methyl-1-oxobutan-2-yl) (methyl) carbamoyl) piperazine-1-carboxylate

Into a 100-mL vial were added benzyl methyl-L-valinate (2.0 g, 9.038 mmol) and triphosgene (0.89 g, 2.982 mmol) in DCM (30 mL) followed by pyridine (2.14 g, 27.113 mmol) in portions at 0° C. under an N₂ atmosphere. The mixture was stirred for 2 h at room temperature. The crude product was used in the next step directly without further purification. Then, the resulting mixture was added to tert-butyl piperazine-1-carboxylate (2.22 g, 11.912 mmol) in DCM (25 mL) and Et₃N (2.78 g, 27.489 mmol) in portions at room temperature under an N₂ atmosphere. The resulting mixture was concentrated under reduced pressure. The residue was purified by silica gel column chromatography, (30% EtOAc/pet. ether) to afford the desired product (3.6 g, 90.6% yield). LCMS (ESI) m/z: [M+H] calcd for C₂₃H₃₅N₃O₅: 434.26; found 434.2.

### Step 2: Synthesis of N-(4-(tert-butoxycarbonyl) piperazine-1-carbonyl)-N-methyl-L-valine

Into a 100-mL vial were added tert-butyl(S)-4-((1-(benzyloxy)-3-methyl-1-oxobutan-2-yl) (methyl) carbamoyl) piperazine-1-carboxylate (2.95 g, 6.804 mmol) and Pd/C (1.48 g) in THF (25 mL). the reaction was stirred for overnight at room temperature under a hydrogen atmosphere. The resulting mixture was filtered, the filter cake was washed with EtOAc (3×50 mL), and the combined organic layers were concentrated under reduced pressure to afford the desired product (2.4 g, crude). LCMS (ESI) m/z: [M+H] calcd for C₁₆H₂₉N₃O₅: 344.21; found 344.4.

### Step 3: Synthesis of tert-butyl 4-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) piperazine-1-carboxylate

Into a 50-mL vial was added (6³S,4S)-4-amino-1¹-ethyl-1²-(2-((S)-1-methoxyethyl) pyridin-3-yl)-10, 10-dimethyl-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (1.0 g, 1.256 mmol) and N-(4-(tert-butoxycarbonyl) piperazine-1-carbonyl)-N-methyl-L-valine (647.04 mg, 1.884 mmol) in DMF (8 mL) followed by HATU (668.63 mg, 1.758 mmol) and DIPEA (811.69 mg, 6.280 mmol) in portions at room temperature. The resulting mixture was extracted with EtOAc (3×50 mL). The combined organic layers were washed with brine (2×20 mL), dried over Na₂SO₄, filtered, and concentrated under reduced pressure. The residue was purified by silica gel column chromatography (50% EtOAc/pet. ether) to afford the desired product (1.08 g, 76.7% yield). LCMS (ESI) m/z: [M+H] calcd for C₆₂H₉₂N₈O₉Si: 1121.68; found 1122.0.

### Step 4: Synthesis of N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperazine-1-carboxamide

Into a 100-mL vial was added tert-butyl 4-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) carbamoyl) piperazine-1-carboxylate (1.08 g, 0.963 mmol) and TFA (3.0 mL, 40.39 mmol) in DCM (12 mL). The reaction was stirred for 2 h at room temperature under an N₂ atmosphere. The resulting mixture was concentrated under reduced pressure to afford the desired product (907 mg, crude). LCMS (ESI) m/z: [M+Na] calcd for C₅₇H₈₃N₈O₇Si: 1042.61; found 1043.9.

### Step 5: Synthesis of N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-4-((R)-1-tritylaziridine-2-carbonyl) piperazine-1-carboxamide

Into a 40-mL vial was added N-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperazine-1-carboxamide (400.0 mg, 0.392 mmol) and (R)-1-tritylaziridine-2-carboxylic acid (193.49 mg, 0.587 mmol) in DMF (3.5 mL) followed by HATU (208.46 mg, 0.548 mmol) and DIPEA (253.06 mg, 1.958 mmol) in portions at room temperature under an N₂ atmosphere. The resulting mixture was extracted with EtOAc (3×60 mL) and the combined organic layers were washed with brine (2×10 mL), dried over Na₂SO₄, filtered, and concentrated under reduced pressure. The residue was purified by silica gel column chromatography, (50% EtOAc/pet. ether) to afford the desired product (367 mg, 70.3% yield). LCMS (ESI) m/z: [M+H-TIPS] calcd for C₇₉H₁₀₁N₉₀₈Si: 1176.63; found 1176.2.

### Step 6: Synthesis of N-((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-4-((R)-1-tritylaziridine-2-carbonyl) piperazine-1-carboxamide

Into a 100-mL vial was added N-((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-4-((R)-1-tritylaziridine-2-carbonyl) piperazine-1-carboxamide (161.0 mg, 0.121 mmol) and CsF (91.75 mg, 0.604 mmol) in DMF (1.5 mL). The reaction was stirred for 2 h at room temperature and was then extracted with EtOAc (3×20 mL). The combined organic layers were washed with brine (2×10 mL), dried over Na₂SO₄, filtered, and concentrated under reduced pressure. The residue was purified by Prep-TLC(50% EtOAc/pet. ether) to afford the desired product (101 mg, 71.1% yield). LCMS (ESI) m/z: [M+H] calcd for C₇₀H₈₁N₉O₈: 1176.62; found 1176.9.

### Step 7: Synthesis of 4-((R)-aziridine-2-carbonyl)-N-((2S)-1-(((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methylpiperazine-1-carboxamide

Into a 40-mL vial was added N-((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl)-N-methyl-4-((R)-1-tritylaziridine-2-carbonyl) piperazine-1-carboxamide (101.0 mg, 0.086 mmol) and Et₃SiH (49.91 mg, 0.429 mmol) in DCM (2.0 mL) was added TFA (48.94 mg, 0.429 mmol) in portions at room temperature under an N₂ atmosphere. The mixture was basified to pH 8 with DIPEA. The crude product was purified by Prep-HPLC to afford the desired product (29.6 mg, 36.9% yield). LCMS (ESI) m/z: [M+H] calcd for C₅₁H₆₇N₉O₈: 934.51; found 934.3.

### Example 175. Synthesis of (2S)-2-((S)-7-((R)-aziridine-2-carbonyl)-1-oxo-2,7-diazaspiro[4.4]nonan-2-yl)-2-cyclopentyl-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide

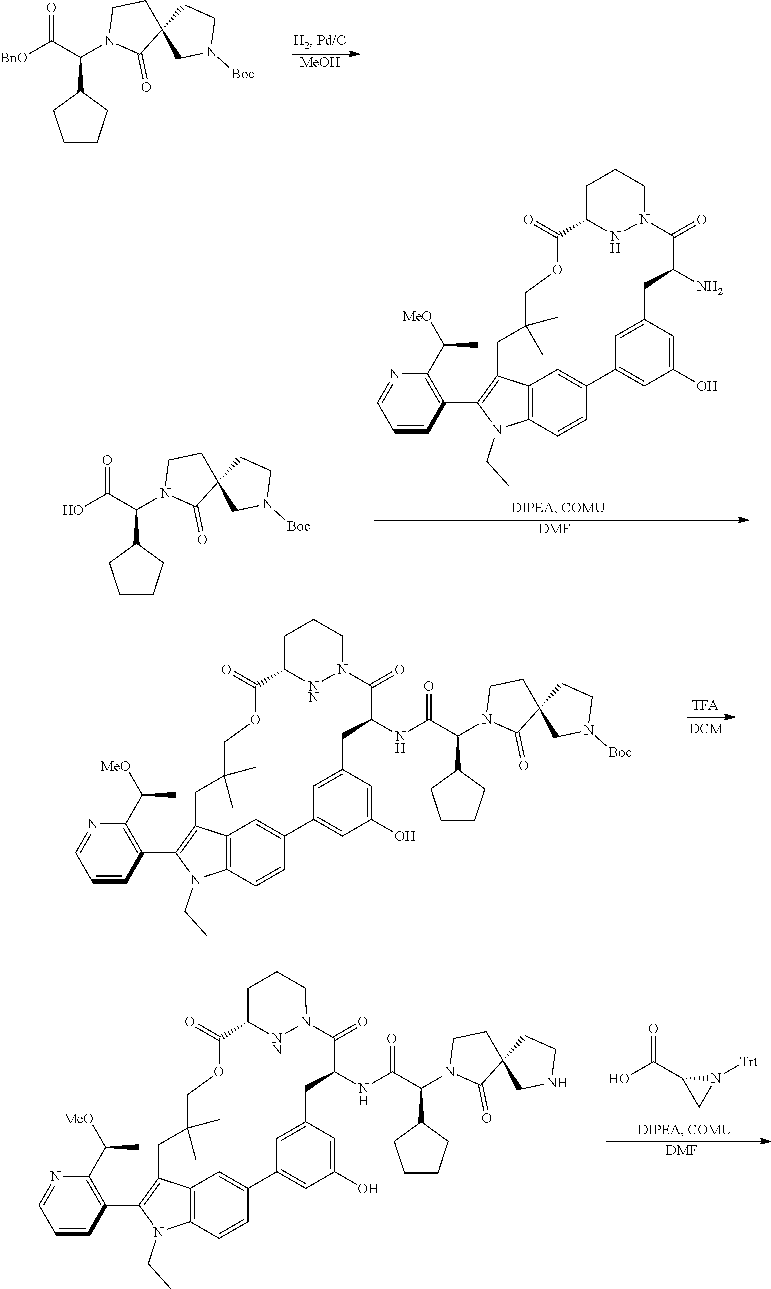

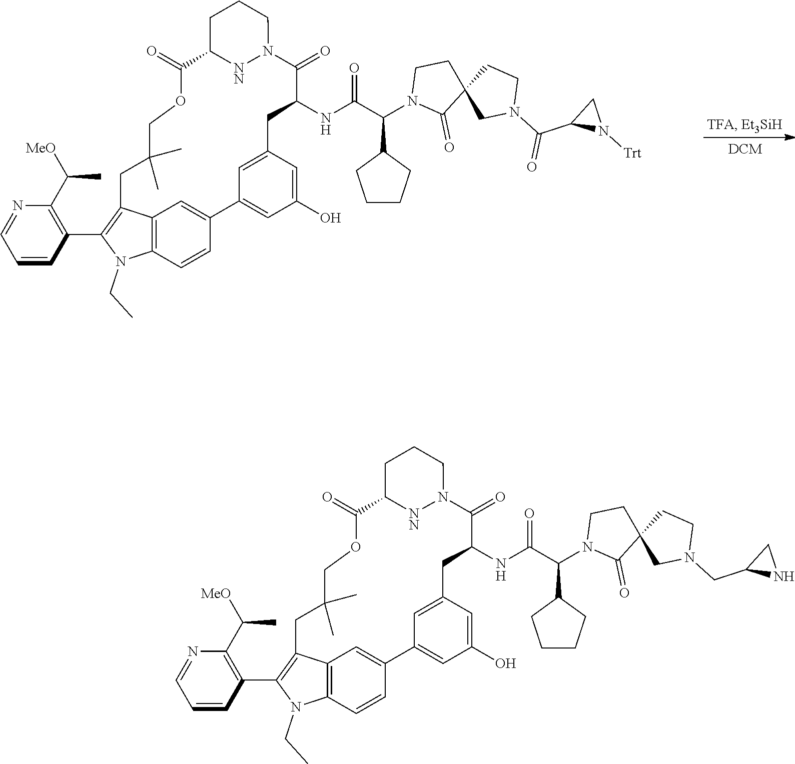

### Step 1: Synthesis of(S)-2-((R)-7-(tert-butoxycarbonyl)-1-oxo-2,7-diazaspiro[4.4]nonan-2-yl)-2-cyclopentylacetic acid

To a solution stirred solution of tert-butyl (R)-7-((S)-2-(benzyloxy)-1-cyclopentyl-2-oxoethyl)-6-oxo-2,7-diazaspiro[4.4]nonane-2-carboxylate (1.0 g, 2.19 mmol) in MeOH (10 mL) at 0° C. was added Pd/C (200 mg). The resulting mixture was stirred for 1 h at room temperature under a hydrogen atmosphere, filtered, and the filter cake washed with MeOH (5×10 mL). The filtrate was concentrated under reduced pressure to afford the desired product (895 mg, crude) which was used without further purification. LCMS (ESI) m/z: [M+H] calcd for C₁₉H₃₀N₂O₅: 376.23; found 367.1.

### Step 2: Synthesis of tert-butyl (5R)-7-((1S)-1-cyclopentyl-2-(((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl)-6-oxo-2,7-diazaspiro[4.4]nonane-2-carboxylate

To a stirred solution of (6³S,4S)-4-amino-1¹-ethyl-25-hydroxy-1²-(2-((S)-1-methoxyethyl) pyridin-3-yl)-10,10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (702 mg, 1.10 mmol) and DIPEA (1.91 mL, 1.10 mmol) in DMF (500 mL) at 0° C. was added(S)-2-((R)-7-(tert-butoxycarbonyl)-1-oxo-2,7-diazaspiro[4.4]nonan-2-yl)-2-cyclopentylacetic acid (523 mg, 1.43 mmol) and COMU (517 mg, 1.21 mmol). After 1 h at room temperature the reaction mixture was diluted with H₂O (150 mL). The aqueous layer was extracted with EtOAc (3×50 mL) and the combined organic layers were washed with brine, dried with Na₂SO₄, filtered, and concentrated under reduced pressure. Purification by normal phase chromatography (40% EtOAc/pet. ether) afforded the desired product (978 mg, 90.2% yield). LCMS (ESI) m/z: [M+H] calcd for C₅₆H₇₃N₇O₉: 988.56; found 988.7.

### Step 3: Synthesis of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-((S)-1-oxo-2,7-diazaspiro[4.4]nonan-2-yl) acetamide

To a stirred solution of tert-butyl (5R)-7-((1S)-1-cyclopentyl-2-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl)-6-oxo-2,7-diazaspiro[4.4]nonane-2-carboxylate (300 mg, 0.304 mmol) in DCM (3.0 mL) at 0° C. was added TFA (1.5 mL). The resulting mixture was stirred for 30 min at room temperature. The reaction mixture was then diluted with toluene (2 mL) and concentrated under reduced pressure three times to afford the desired product (270 mg, crude) which was used without further purification. LCMS (ESI) m/z: [M+H] calcd for C₅₁H₆₅N₇O₇: 888.50; found 888.5.

### Step 4: Synthesis of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-61, 62,63,64,65,66-hexahydro-1¹H-8-oxa-1 (5,3)-indola-6 (1,3)-pyridazina-2 (1,3)-benzenacycloundecaphane-4-yl)-2-((S)-1-oxo-7-((R)-1-tritylaziridine-2-carbonyl)-2,7-diazaspiro[4.4]nonan-2-yl) acetamide

To a stirred solution of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-((S)-1-oxo-2,7-diazaspiro[4.4]nonan-2-yl) acetamide (270 mg, 0.304 mmol) and DIPEA (0.53 mL, 3.0 mmol) in DMF (3.0 mL) at 0° C. was added (R)-1-tritylaziridine-2-carboxylic acid (130 mg, 0.395 mmol) and COMU (143 mg, 0.334 mmol). After 1 h at room temperature the reaction mixture was diluted with H₂O (30 mL). The aqueous layer was extracted with EtOAc (3×3 mL) and the combined organic layers were washed with brine, dried with Na₂SO₄, filtered, and concentrated under reduced pressure. Purification by prep-TLC(5% MeOH/DCM) afforded the desired product (332 mg, 91.1% yield). LCMS (ESI) m/z: [M+H] calcd for C₇₃H₈₂N₈O₈: 1199.64; found 1199.7.

### Step 5: Synthesis of (2S)-2-((S)-7-((R)-aziridine-2-carbonyl)-1-oxo-2,7-diazaspiro[4.4]nonan-2-yl)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide

To a stirred solution of (2S)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-((S)-1-oxo-7-((R)-1-tritylaziridine-2-carbonyl)-2,7-diazaspiro[4.4]nonan-2-yl) acetamide (309 mg, 0.258 mmol) in DCM (3.0 mL) at 0° C. was added Et₃SiH (164 mL, 1.03 mmol) and TFA (79 mL, 1.03 mmol). After 30 min the reaction mixture was basified to pH 8 with DIPEA and concentrated under reduced pressure. Purification by reverse phase chromatography (30→60% MeCN/H₂O) afforded the desired product (36 mg, 14.2% yield). LCMS (ESI) m/z: [M+H] calcd for C₅₄H₆₈N₈O₈: 957.53; found 957.3.

### Example 214. Synthesis of (2S)-2-cyclopentyl-2-((S)-7-((2R,3R)-3-cyclopropylaziridine-2-carbonyl)-1-oxo-2,7-diazaspiro[4.4]nonan-2-yl)-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide

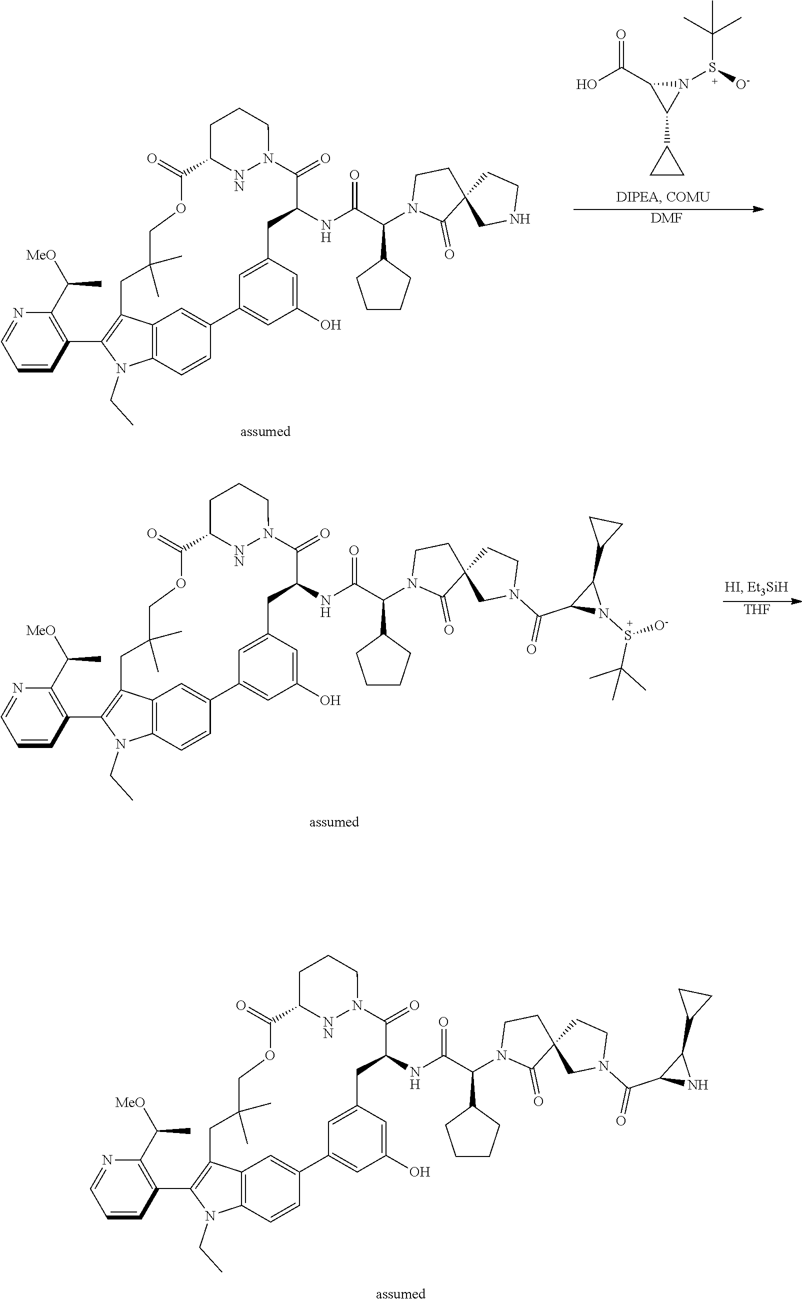

### Step 1: Synthesis of (2S)-2-((5S)-7-((2R,3R)-1-(tert-butylsulfinyl)-3-cyclopropylaziridine-2-carbonyl)-1-oxo-2,7-diazaspiro[4.4]nonan-2-yl)-2-cyclopentyl-N-((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide

To a stirred solution of (2S)-2-cyclopentyl-N-((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-((S)-1-oxo-2,7-diazaspiro[4.4]nonan-2-yl) acetamide (270 mg, 0.30 mmol) in DMF (3.0 mL) at 0° C. was added DIPEA (530 μL, 3.0 mmol) and (2R,3R)-1-(tert-butylsulfinyl)-3-cyclopropylaziridine-2-carboxylic acid (105 mg, 0.46 mmol) followed by COMU (140 mg, 0.33 mmol). The resulting mixture was stirred for 1 h at room temperature and was then diluted with H₂O (30 mL). The reaction mixture was extracted into EtOAc (3×7 mL). The combined organic layers were washed with brine (3×10 mL), dried over Na₂SO₄, filtered, and concentrated under reduced pressure. Purification by Prep-TLC (6% MeOH/DCM) afforded the desired product (237 mg, 71% yield). LCMS (ESI) m/z: [M+H] calcd for C₆₁H₈₀N₈O₉S: 1101.58; found 1101.3.

### Step 2: Synthesis of (2S)-2-cyclopentyl-2-((S)-7-((2R,3R)-3-cyclopropylaziridine-2-carbonyl)-1-oxo-2,7-diazaspiro[4.4]nonan-2-yl)-N-((63 S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide

To a stirred solution of (2S)-2-((5S)-7-((2R,3R)-1-(tert-butylsulfinyl)-3-cyclopropylaziridine-2-carbonyl)-1-oxo-2,7-diazaspiro[4.4]nonan-2-yl)-2-cyclopentyl-N-((6³S,4S)-1¹-ethyl-25-hydroxy-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) acetamide (230 mg, 0.21 mmol) in THF (2.5 mL) at 0° C. was added Et₃SiH (130 μL, 0.83 mmol) and HI (125 μL, 0.41 mmol, 57% in H₂O). The resulting mixture was stirred for 30 min at room temperature then cooled to 0° C. and neutralized to pH 8. The mixture was concentrated under reduced pressure. Purification by Prep-TLC(8.3% MeOH/DCM) afforded the desired product (46 mg, 21% yield). LCMS (ESI) m/z: [M+H] calcd for C₅₇H₇₂N₈O₈: 997.55; found 997.2.

### Example 209. Synthesis of (2R)—N-(2-(((1S)-1-cyclopentyl-2-(((6³S,4S)-1¹-ethyl-25-hydroxy-12-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methylaziridine-2-carboxamide

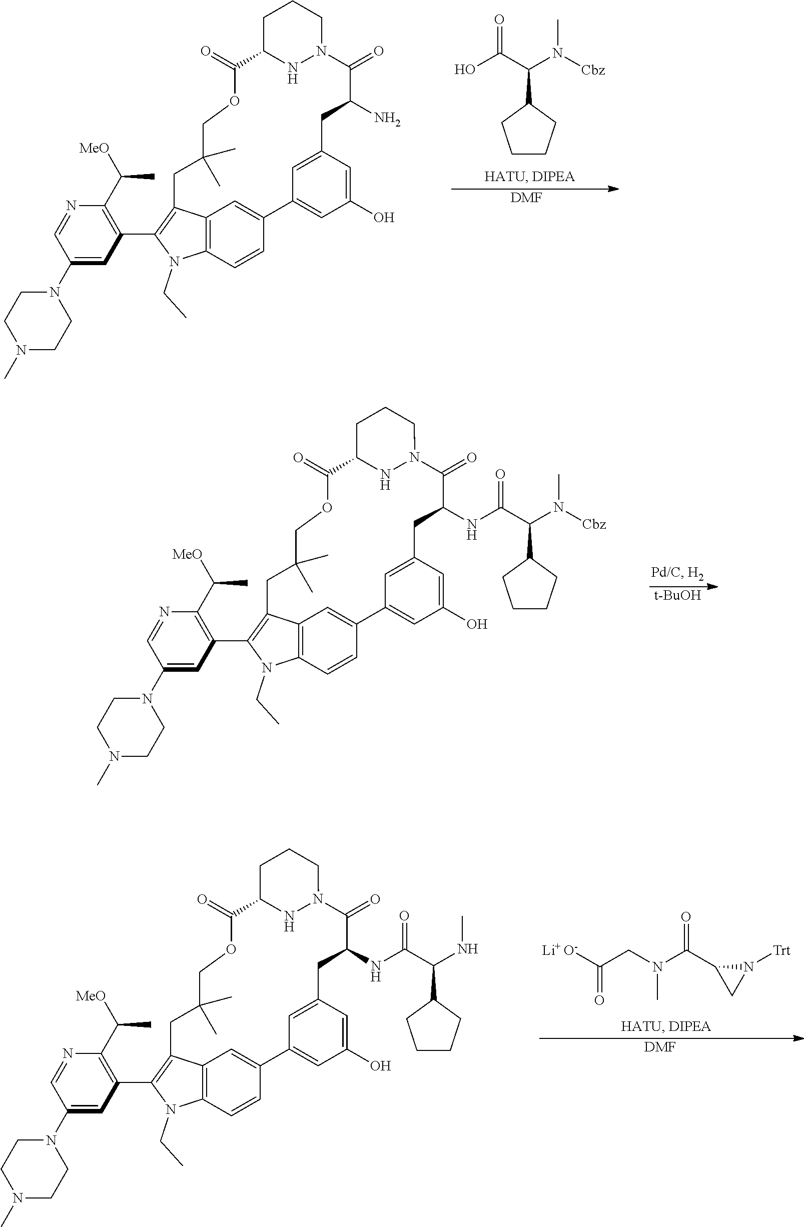

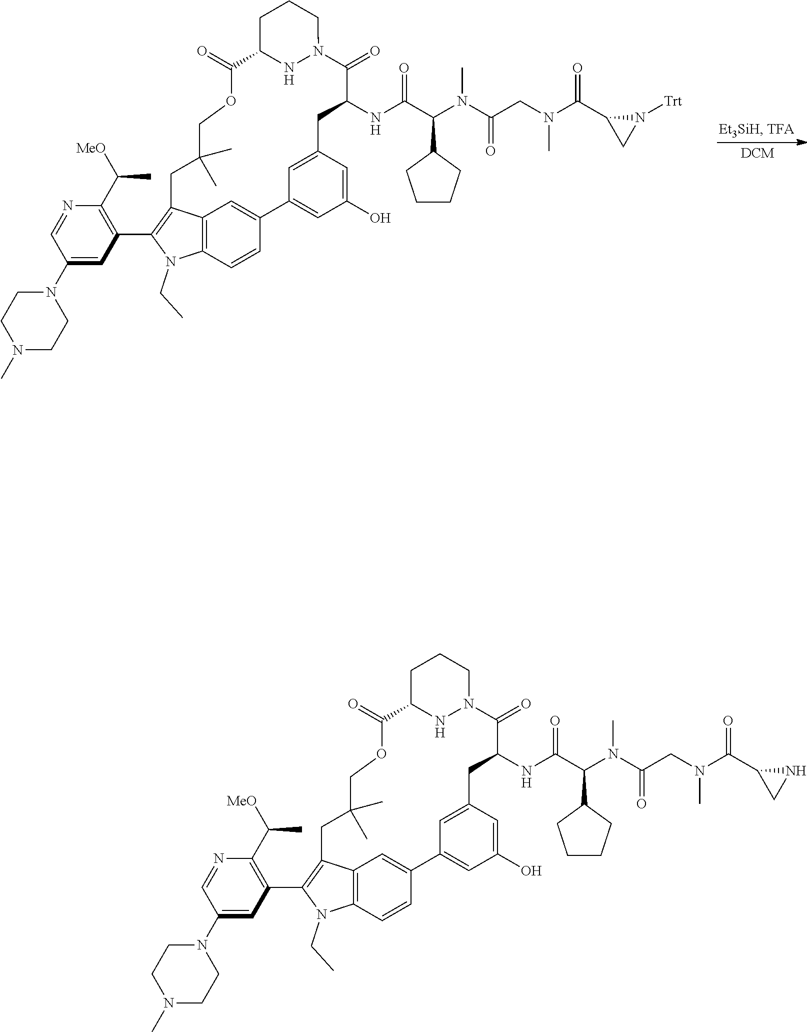

### Step 1: Synthesis of benzyl ((1S)-1-cyclopentyl-2-(((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) carbamate

To a stirred solution of (63S, 4S)-4-amino-1¹-ethyl-25-hydroxy-1²-(2-((S)-1-methoxyethyl)-5-(4-methylpiperazin-1-yl) pyridine-3-yl)-10, 10-dimethyl-6¹,6²,6³,6⁴,6⁵,6⁶-hexahydro-1¹H-8-oxa-1 (5,3)-indola-6 (1,3)-pyridazina-2 (1,3)-benzenacycloundecaphane-5,7-dione (490 mg, 0.664 mmol) and (S)-2-(((benzyloxy) carbonyl) (methyl) amino)-2-cyclopentylacetic acid (232 mg, 0.797 mmol) in DMF (5 mL) at 0° C. was added DIPEA (1.19 mL, 6.64 mmol) and HATU (303 mg, 0.797 mmol). The resulting mixture was stirred for 1 h at room temperature and then diluted with H₂O (20 mL). The aqueous phase was extracted with EtOAc (3×20 mL) and the combined organic layers were washed with brine, dried over Na₂SO₄, filtered, and concentrated under reduced pressure. Purification by reverse phase chromatography (0→100% MeCN/H₂O, 0.1% NH₄HCO₃) afforded the desired product (420 mg, 59.4% yield). LCMS (ESI) m/z: [M+H] calcd for C₅₈H₇₄N₈O₈: 1011.57; found 1011.6.

### Step 2: Synthesis of (2S)-2-cyclopentyl-N-((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(methylamino) acetamide

To a stirred solution of benzyl ((1S)-1-cyclopentyl-2-(((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) carbamate (450 mg, 0.445 mmol) in t-BuOH (10 mL) was added Pd/C (90 mg). The resulting mixture was warmed to 40° C. overnight under a hydrogen atmosphere, then filtered and the filter cake washed with MeOH. The filtrate was concentrated under reduced pressure to afford the desired product (420 mg, crude) which was used without further purification. LCMS (ESI) m/z: [M+H] calcd for C₅₀H₆₈N₈O₆: 877.54; found 877.5.

### Step 3: Synthesis of (2R)—N-(2-(((1S)-1-cyclopentyl-2-(((6³S,4S)-1¹-ethyl-25-hydroxy-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methyl-1-tritylaziridine-2-carboxamide

To a stirred solution of (2S)-2-cyclopentyl-N-((6³S,4S)-1¹-ethyl-25-hydroxy-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-2-(methylamino) acetamide (130 mg, 0.148 mmol) and lithium (R)—N-methyl-N-(1-tritylaziridine-2-carbonyl) glycinate (78.3 mg, 0.193 mmol) in DMF (2 mL) at 0° C. was added DIPEA (264 mL, 1.48 mmol) and HATU (68 mg, 0.178 mmol). The resulting mixture was stirred for 1 h at room temperature and then diluted with H₂O (20 mL). The aqueous phase was extracted with EtOAc (3×10 mL) and the combined organic layers were washed with H₂O, dried with Na₂SO₄, filtered, and concentrated under reduced pressure. Purification by prep-TLC(10% MeOH/DCM) afforded the desired product (100 mg, 50.9% yield). LCMS (ESI) m/z: [M+Na] calcd for C₇₅H₉₀N₁₀O₈: 1281.69; found 1281.9.

### Step 4: Synthesis of (2R)—N-(2-(((1S)-1-cyclopentyl-2-(((6³S,4S)-1¹-ethyl-25-hydroxy-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methylaziridine-2-carboxamide

To a stirred solution of (2R)—N-(2-(((1S)-1-cyclopentyl-2-(((6³S,4S)-1¹-ethyl-25-hydroxy-12-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-2-oxoethyl) (methyl) amino)-2-oxoethyl)-N-methyl-1-tritylaziridine-2-carboxamide (100 mg, 0.079 mmol) in DCM (1.0 mL) at 0° C. was added Et₃SiH (51 mL, 0.318 mmol) and TFA (24 mL, 0.318 mmol). After 30 min the reaction mixture was basified to pH 8 with DIPEA and concentrated under reduced pressure. Purification by reverse phase chromatography (30→55% MeCN/H₂O) afforded the desired product (14 mg, 16.5% yield). LCMS (ESI) m/z: [M+H] calcd for C₅₆H₇₆N₁₀O₈: 1017.59; found 1017.6.

### Example 268. Synthesis of (2R)—N-(2-(((2S)-1-(((6³S,4S)-1¹-ethyl-25-hydroxy-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(3-(2-oxopyrrolidin-1-yl) propyl) aziridine-2-carboxamide

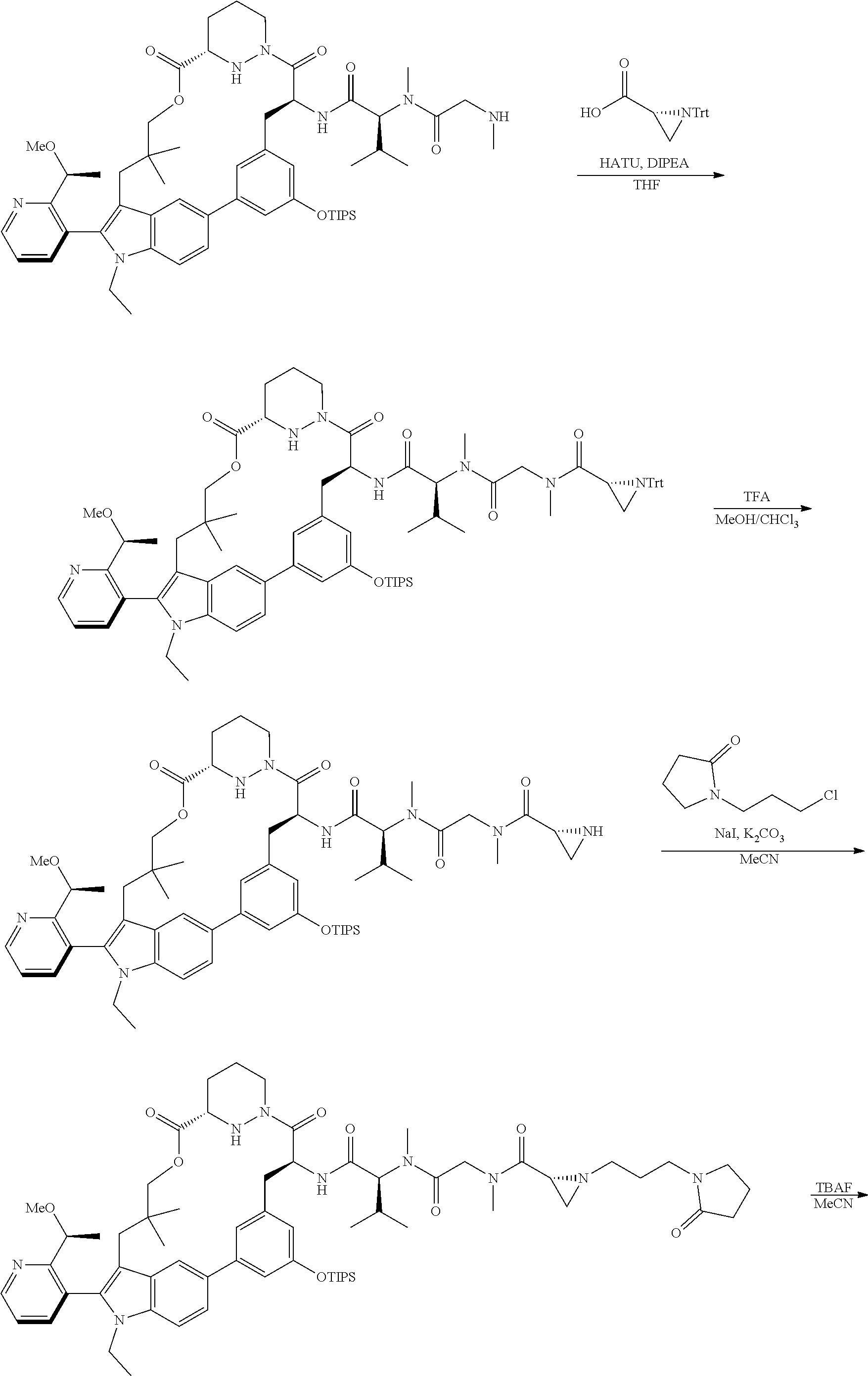

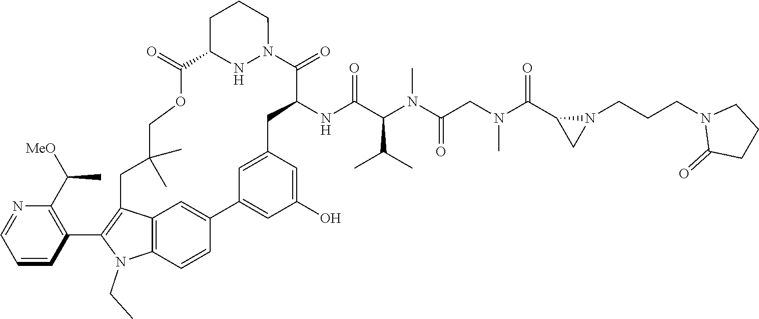

### Step 1: Synthesis of (2R)—N-(2-(((2S)-1-(((6³S,4S)-1¹-ethyl-1²-(2-((S)-1-methoxyethyl) pyridin-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide

To a solution of (1S)-1-tritylaziridine-2-carboxylic acid (537.6 mg, 1.63 mmol), (2S)—N-((6³S,4S)-1¹-ethyl-1²-(2-((S)-1-methoxyethyl) pyridin-3-yl)-10, 10-dimethyl-5,7-dioxo-25-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl)-3-methyl-2-(N-methyl-2-(methylamino) acetamido) butanamide (800 mg, 0.816 mmol) in THF (8 mL) was added DIPEA (0.711 mL, 4.08 mmol), HATU (465.4 mg, 1.22 mmol) at 0° C., the reaction was warmed to room temperature and stirred for 2 h. To the reaction was added H₂O (20 mL), the aqueous phase was extracted with DCM (3×30 mL) and the combined organic phases were washed with brine (20 mL), dried over Na₂SO₄, filtered, and concentrated under reduced pressure. The residue was purified by silica gel column chromatography (0→100% EtOAc/pet. ether) to afford the desired product (1 g, 94.9% yield).

### Step 2: Synthesis of (2R)—N-(2-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide

To a solution of (2R)—N-(2-(((2S)-1-(((6³S,4S)-1¹-ethyl-1²-(2-((S)-1-methoxyethyl) pyridine-3 -yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-tritylaziridine-2-carboxamide (1 g, 0.774 mmol) in MeOH (5 mL) and CHCl₃ (5 mL) was added TFA (1.15 mL, 15.48 mmol) at 0° C. The reaction was warmed to room temperature and stirred for 2 h. The reaction mixture was added dropwise to aq. NaHCO₃ (30 mL) at 0° C. Then the pH was adjusted to pH 7-8 with using aq. NaHCO₃ at 0° C. The mixture was extracted with DCM (3×20 mL), dried over Na₂SO₄, filtered, and concentrated under reduced pressure to afford the desired product (960 mg, crude), which was used directly in the next step without further purification.

### Step 3: Synthesis of (2R)—N-(2-(((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(3-(2-oxopyrrolidin-1-yl) propyl) aziridine-2-carboxamide

To a solution of (2R)—N-(2-(((2S)-1-(((6³S,4S)-1¹-ethyl-1²-(2-((S)-1-methoxyethyl) pyridine-3-yl)-10, 10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methylaziridine-2-carboxamide (960 mg, 0.915 mmol) in MeCN (10 mL) was added 1-(3-chloropropyl) pyrrolidin-2-one (887.1 mg, 5.49 mmol), K₂CO₃ (1.14 g, 8.23 mmol), NaI (411.4 mg, 2.74 mmol), the reaction was stirred at 80° C. for 24 h. To the reaction was added H₂O (20 mL), the aqueous phase was extracted with EtOAc (3×20 mL). The combined organic phase was dried over Na₂SO₄, filtered, and concentrated under reduced pressure. The residue was purified by reverse phase chromatography (73→93% MeCN/H₂O, 10 mM NH₄HCO₃) to afford product (80 mg, 7.5% yield). LCMS (ESI) m/z: [M+H] calcd for C₆₅H₉₆N₉O₉Si: 1174.7; found 1174.7.

### Step 4: Synthesis of (2R)—N-(2-(((2S)-1-(((6³S,4S)-1¹-ethyl-25-hydroxy-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(3-(2-oxopyrrolidin-1-yl) propyl) aziridine-2-carboxamide

To a solution of (2R)—N-(2-(((2S)-1-(((6³S,4S)-1¹-ethyl-1²-(2-((S)-1-methoxyethyl) pyridine-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1 (5,3)-indola-6 (1,3)-pyridazina-2 (1,3)-benzenacycloundecaphane-4-yl) amino)-3-methyl-1-oxobutan-2-yl) (methyl) amino)-2-oxoethyl)-N-methyl-1-(3-(2-oxopyrrolidin-1-yl) propyl) aziridine-2-carboxamide (80 mg, 0.068 mmol) in THF (1 mL) was added TBAF (1 M, 0.082 mL). The reaction was stirred for 1 h and then was added to H₂O (10 mL), the aqueous phase was extracted with EtOAc (3×10 mL). The combined organic phase was dried over Na₂SO₄, filtered, and concentrated under reduced pressure. The resulting residue was purified by reverse phase chromatography (25→65% MeCN/H₂O, 10 mM NH₄HCO₃ to afford the desired product (42 mg, 60.4% yield). LCMS (ESI) m/z: [M+H] calcd for C₅₆H₇₆N₉O₉: 1018.6; found 1018.5.

The following table of compounds (Table 4) were prepared using the aforementioned methods or variations thereof, as is known to those of skill in the art.

TABLE 4
Exemplary Compounds Prepared by Methods of the
Present Invention

| | Molecular | | Observed MW |
|---|---|---|---|
| Ex# | Formula | Calculated MW | LCMS (ESI) m/z |
| 1 | C₅₀H₆₄N₈O₈ | [M + H] = 905.49 | [M + H] = 905.7 |
| 2 | C₅₀H₆₄N₈O₈ | [M + H] = 905.49 | [M + H] = 905.7 |
| 3 | C₅₁H₆₆N₈O₈ | [M + H] = 919.51 | [M + H] = 919.7 |
| 4 | C₅₁H₆₆N₈O₈ | [M + H] = 919.51 | [M + H] = 919.6 |
| 5 | C₄₉H₆₂N₈O₈ | [M + H] = 891.48 | [M + H] = 891.7 |
| 6 | C₄₉H₆₂N₈O₈ | [M + H] = 891.48 | [M + H] = 891.7 |
| 7 | C₄₈H₆₂N₈O₈ | [M + H] = 879.48 | [M + H] = 879.7 |
| 8 | C₄₈H₆₂N₈O₈ | [M + H] = 879.48 | [M + H] = 879.7 |
| 9 | C₅₆H₆₈N₈O₈ | [M + H] = 969.52 | [M + H] = 969.5 |
| 10 | C₅₆H₆₈N₈O₈ | [M + H] = 969.52 | [M + H] = 969.5 |
| 11 | C₅₄H₆₆N₈O₈ | [M + H] = 955.51 | [M + H] = 955.7 |
| 12 | C₅₄H₆₆N₈O₈ | [M + H] = 955.51 | [M + H] = 955.7 |
| 13 | C₄₉H₆₄N₈O₁₀S | [M + H] = 957.45 | [M + H] = 957.5 |
| 14 | C₅₀H₆₄N₈O₉ | [M + H] = 921.49 | [M + H] = 921.5 |
| 15A | C₅₃H₆₄N₈O₇ | [M + H] = 925.50 | [M + H] = 925.5 |
| 15B | C₅₃H₆₄N₈O₇ | [M + H] = 925.50 | [M + H] = 925.5 |
| 16A | C₅₀H₆₆N₈O₆S | [M + H] = 907.49 | [M + H] = 907.5 |
| 16B | C₅₀H₆₆N₈O₆S | [M + H] = 907.49 | [M + H] = 907.5 |
| 17A | C₅₃H₇₁N₉O₇ | [M + H] = 946.56 | [M + H] = 946.6 |
| 17B | C₅₃H₇₁N₉O₇ | [M + H] = 946.56 | [M + H] = 946.6 |
| 18A | C₅₄H₆₆N₈O₇ | [M + H] = 940.52 | [M + H] = 939.6 |
| 18B | C₅₄H₆₆N₈O₇ | [M + H] = 940.52 | [M + H] = 939.6 |
| 19A | C₅₃H₆₄N₈O₆ | [M + H] = 909.50 | [M + H] = 909.6 |
| 19B | C₅₃H₆₄N₈O₆ | [M + H] = 909.50 | [M + H] = 909.5 |
| 20A | C₅₃H₆₄N₈O₇ | [M + H] = 925.50 | [M + H] = 925.5 |
| 20B | C₅₃H₆₄N₈O₇ | [M + H] = 925.50 | [M + H] = 925.5 |
| 21A | C₄₉H₆₄N₈O₆ | [M + H] = 861.50 | [M + H] = 861.5 |
| 21B | C₄₉H₆₄N₈O₆ | [M + H] = 861.50 | [M + H] = 861.5 |
| 22A | C₄₉H₆₄N₈O₆ | [M + H] = 861.50 | [M + H] = 861.6 |
| 22B | C₄₉H₆₄N₈O₆ | [M + H] = 861.50 | [M + H] = 861.5 |
| 23A | C₄₉H₆₄N₈O₇ | [M + H] = 877.50 | [M + H] = 877.6 |
| 23B | C₄₉H₆₄N₈O₇ | [M + H] = 877.50 | [M + H] = 877.6 |
| 24A | C₅₄H₆₆N₈O₈ | [M + H] = 955.51 | [M + H] = 955.6 |
| 24B | C₅₄H₆₆N₈O₈ | [M + H] = 955.51 | [M + H] = 955.6 |
| 25 | C₄₈H₆₃ClN₈O₈ | [M + H] = 915.45 | [M + H] = 915.7 |
| 26 | C₄₆H₆₀ClN₇O₇ | [M + H] = 858.43 | [M + H] = 858.7 |
| 27 | C₄₈H₆₂N₈O₈ | [M + H] = 879.48 | [M + H] = 879.4 |
| 28 | C₄₇H₆₀N₈O₈ | [M + H] = 865.46 | [M + H] = 865.7 |
| 29 | C₄₅H₅₇N₇O₇ | [M + H] = 808.44 | [M + H] = 808.8 |
| 30 | C₄₆H₅₉N₇O₇ | [M + H] = 822.46 | [M + H] = 822.4 |
| 31 | C₅₀H₆₃N₇O₉ | [M + H] = 906.48 | [M + H] = 906.7 |
| 32A | C₅₄H₆₆N₈O₇ | [M + H] = 940.18 | [M + H] = 939.6 |
| 32B | C₅₄H₆₆N₈O₇ | [M + H] = 940.18 | [M + H] = 939.6 |
| 33 | C₄₈H₆₂N₈O₈ | [M + H] = 879.48 | [M + H] = 878.5 |
| 34 | C₅₀H₆₄N₈O₉ | [M + H] = 921.49 | [M + H] = 921.5 |
| 35 | C₅₀H₆₄N₈O₉ | [M + H] = 921.49 | [M + H] = 921.6 |
| 36 | C₄₉H₆₄N₈O₁₀S | [M + H] = 957.45 | [M + H] = 957.5 |
| 37 | C₄₉H₆₄N₈O₁₀S | [M + H] = 957.45 | [M + H] = 957.5 |
| 38 | C₅₀H₆₄N₈O₁₀ | [M + H] = 937.48 | [M + H] = 937.6 |
| 39 | C₅₀H₆₄N₈O₁₀ | [M + H] = 937.48 | [M + H] = 937.5 |
| 40 | C₄₉H₆₄N₈O₈ | [M + H] = 893.49 | [M + H] = 893.7 |
| 41 | C₄₉H₆₄N₈O₈ | [M + H] = 893.49 | [M + H] = 893.8 |
| 42A | C₄₉H₆₄N₈O₈ | [M + H] = 893.49 | [M + H] = 893.7 |
| 42B | C₄₉H₆₄N₈O₈ | [M + H] = 893.49 | [M + H] = 893.7 |
| 43A | C₅₁H₆₆N₈O₈ | [M + H] = 919.51 | [M + H] = 919.8 |
| 43B | C₅₁H₆₆N₈O₈ | [M + H] = 919.51 | [M + H] = 919.7 |
| 44 | C₅₁H₆₇N₉O₈ | [M + H] = 934.52 | [M + H] = 934.8 |
| 45 | C₅₁H₆₇N₉O₈ | [M + H] = 934.52 | [M + H] = 934.7 |
| 46 | C₅₁H₆₁N₉O₈ | [M + H] = 928.47 | [M + H] = 928.7 |
| 47A | C₅₁H₆₁N₉O₈ | [M + H] = 928.47 | [M + H] = 928.7 |
| 47B | C₅₁H₆₁N₉O₈ | [M + H] = 928.47 | [M + H] = 928.7 |
| 48 | C₅₄H₇₂N₈O₁₁S | [M + H] = 1041.51 | [M + H] = 1041.8 |
| 49 | C₅₀H₆₄N₈O₁₀ | [M + H] = 937.48 | [M + H] = 938.6 |
| 50 | C₅₄H₇₂N₈O₁₁S | [M + H] = 1041.51 | [M + H] = 1041.6 |
| 51 | C₅₀H₆₄N₈O₁₀ | [M + H] = 937.48 | [M + H] = 937.8 |
| 52 | C₅₅H₇₄N₈O₉S | [M + H] = 1023.54 | [M + H] = 1023.8 |
| 53 | C₅₃H₇₂N₈O₉S | [M + Na] = 1019.50 | [M + Na] = 1019.8 |
| 54 | C₅₃H₇₂N₈O₉S | [M + H] = 997.52 | [M + H] = 997.7 |
| 55 | C₅₅H₆₄N₈O₉ | [M + H] = 1003.47 | [M + H] = 1003.8 |
| 56 | C₅₁H₆₈N₈O₉S | [M + Na] = 991.47 | [M + Na] = 991.7 |
| 57 | C₅₁H₆₈N₈O₉S | [M + H] = 969.49 | [M + H] = 969.8 |
| 58 | C₅₃H₆₈N₈O₈ | [M + H] = 945.52 | [M + H] = 945.8 |
| 59 | C₅₂H₆₆N₈O₈ | [M + H] = 931.51 | [M + H] = 931.8 |
| 60 | C₄₆H₅₉N₇O₇ | [M + H] = 822.46 | [M + H] = 822.7 |
| 61A | C₅₅H₆₈N₈O₈ | [M + H] = 969.52 | [M + H] = 969.6 |
| 61B | C₅₅H₆₈N₈O₈ | [M + H] = 969.52 | [M + H] = 969.6 |
| 62A | C₅₅H₆₈N₈O₈ | [M + H] = 955.51 | [M + H] = 955.5 |
| 62B | C₅₅H₆₈N₈O₈ | [M + H] = 955.51 | [M + H] = 955.5 |
| 63 | C₅₄H₆₆N₈O₇ | [M + H] = 939.51 | [M + H] = 939.6 |
| 64A | C₅₅H₆₈N₈O₇ | [M + H] = 953.53 | [M + H] = 953.6 |
| 64B | C₅₅H₆₈N₈O₇ | [M + H] = 953.53 | [M + H] = 953.6 |
| 65A | C₅₅H₆₆N₈O₇ | [M + H] = 951.51 | [M + H] = 951.7 |
| 65B | C₅H₆N₈O₇ | [M + H] = 951.51 | [M + H] = 951.6 |
| 66 | C₅₄H₆₇N₉O₆ | [M + H] = 938.53 | [M + H] = 938.5 |
| 67 | C₅₆H₆₉N₉O₇ | [M + H] = 980.54 | [M + H] = 980.1 |
| 68A | C₅₂H₆₄N₈O₆S | [M + H] = 929.47 | [M + H] = 929.5 |
| 68B | C₅₂H₆₄N₈O₆S | [M + H] = 929.47 | [M + H] = 929.5 |
| 69A | C₅₁H₆₈N₈O₇ | [M + H] = 891.51 | [M + H] = 891.5 |
| 69B | C₅₁H₆₈N₈O₇ | [M + H] = 891.51 | [M + H] = 891.5 |
| 70A | C₅₆H₆₈N₈O₇ | [M + H] = 965.53 | [M + H] = 965.6 |
| 70B | C₅₆H₆₈N₈O₇ | [M + H] = 965.53 | [M + H] = 965.6 |
| 71A | C₅₆H₆₈N₈O₇ | [M + H] = 965.53 | [M + H] = 965.5 |
| 71B | C₅₆H₆₈N₈O₇ | [M + H] = 965.53 | [M + H] = 965.5 |
| 72A | C₅₁H₆₆N₈O₇ | [M + H] = 903.51 | [M + H] = 903.5 |
| 72B | C₅₁H₆₆N₈O₇ | [M + H] = 903.51 | [M + H] = 903.6 |
| 73 | C₅₅H₆₈N₈O₇ | [M + H] = 953.53 | [M + H] = 953.6 |
| 74 | C₅₀H₆₆N₈O₇ | [M + H] = 891.51 | [M + H] = 891.6 |
| 75 | C₅₅H₆₈N₈O₈ | [M + H] = 969.52 | [M + H] = 969.9 |
| 76 | C₅₈H₇₂N₈O₇ | [M + H] = 993.56 | [M + H] = 993.5 |
| 77 | C₅₇H₇₀N₈O₇ | [M + H] = 979.54 | [M + H] = 979.5 |
| 78 | C₅₂H₆₈N₈O₇ | [M + H] = 917.53 | [M + H] = 917.5 |
| 79A | C₅₀H₆₆N₈O₆ | [M + H] = 875.52 | [M + H] = 875.6 |
| 79B | C₅₀H₆₆N₈O₆ | [M + H] = 875.52 | [M + H] = 875.6 |
| 80 | C₅₀H₆₆N₈O₆ | [M + H] = 875.52 | [M + H] = 875.6 |
| 81 | C₅₄H₆₆N₈O₆ | [M + H] = 923.52 | [M + H] = 923.5 |
| 82A | C₅₄H₆₆N₈O₆ | [M + H] = 923.52 | [M + H] = 923.6 |
| 82B | C₅₄H₆₆N₈O₆ | [M + H] = 923.52 | [M + H] = 923.6 |
| 83 | C₅₁H₆₄N₁₀O₈ | [M + H] = 945.50 | [M + H] = 945.6 |
| 84 | C₅₁H₆₄N₁₀O₈ | [M + H] = 945.50 | [M + H] = 945.6 |
| 85 | C₅₁H₆₈N₈O₉ | [M + H] = 937.52 | [M + H] = 837.6 |
| 86 | C₅₂H₆₈N₈O₈ | [M + H] = 933.52 | [M + H] = 933.7 |
| 87 | C₅₁H₆₂N₈O₇ | [M + H] = 899.48 | [M + H] = 899.5 |
| 88 | C₅₂H₆₈N₈O₈ | [M + H] = 933.52 | [M + H] = 933.6 |
| 89 | C₅₂H₆₈N₈O₈ | [M + H] = 933.52 | [M + H] = 933.6 |
| 90 | C₅₂H₇₀N₈O₉ | [M + H] = 951.53 | [M + H] = 951.6 |
| 91 | C₄₉H₆₆N₈O₉S | [M + H] = 943.48 | [M + H] = 943.6 |
| 92 | C₄₉H₆₆N₈O₉S | [M + H] = 943.48 | [M + H] = 943.5 |
| 93 | C₅₅H₆₈N₈O₉ | [M + H] = 985.52 | [M + H] = 985.7 |
| 94 | C₅₂H₆₆ N₈O₁₀ | [M + H] = 963.50 | [M + H] = 963.6 |
| 95 | C₅₁H₆₆N₈O₈ | [M + H] = 919.51 | [M + H] = 919.5 |
| 96 | C₅₅H₆₈N₈O₉ | [M + H] = 985.52 | [M + H] = 985.7 |
| 97 | C₅₅H₆₈N₈O₉ | [M + H] = 985.52 | [M + H] = 985.7 |
| 98 | C₄₈H₅₈N₈O₉ | [M + H] = 891.44 | [M + H] = 891.6 |
| 99 | C₄₆H₅₉N₇O₇ | [M + H] = 822.46 | [M + H] = 822.6 |
| 100 | C₄₆H₅₉N₇O₇ | [M + H] = 822.46 | [M + H] = 822.6 |
| 101 | C₅₅H₇₄N₈O₉S | [M + Na] = 1045.52 | [M + Na] = 1045.7 |
| 102 | C₅₀H₆₆N₈O₈ | [M + H] = 907.51 | [M + H] = 907.7 |
| 103 | C₅₁H₆₆N₈O₈ | [M + H] = 919.51 | [M + H] = 919.5 |
| 104 | C₅₁H₆N₈O₈ | [M + H] = 919.51 | [M + H] = 919.7 |
| 105 | C₄₉H₆₄N₈O₈ | [M + H] = 893.49 | [M + H] = 893.7 |
| 106 | C₅₁H₆₆N₈O₇ | [M + H] = 903.51 | [M + H] = 930.7 |
| 107 | C₄₉H₆₄N₈O₇ | [M + H] = 877.50 | [M + H] = 877.7 |
| 108 | C₅₂H₇₀N₈O₉S | [M + H] = 983.51 | [M + H] = 983.8 |
| 109 | C₅₁H₆₄N₈O₈ | [M + H] = 917.49 | [M + H] = 917.7 |
| 110 | C₅₄H₆₆N₈O₈ | [M + H] = 955.51 | [M + H] = 955.7 |
| 111 | C₅₂H₆₆ N₈O₁₀ | [M + H] = 963.50 | [M + H] = 963.8 |
| 112 | C₄₆H₅₉N₇O₇ | [M + H] = 822.46 | [M + H] = 822.6 |
| 113 | C₅₂H₆₆N₈O₈ | [M + Na] = 953.49 | [M + Na] = 953.6 |
| 114 | C₅₀H₆₆N₈O₈ | [M + H] = 907.51 | [M + H] = 907.7 |
| 115 | C₅₁H₆₄N₈O₈ | [M + H] = 917.49 | [M + H] = 917.6 |
| 116 | C₅₄H₆₆N₈O₈ | [M + H] = 955.51 | [M + H] = 955.7 |
| 117 | C₅₀H₆₆N₈O₉S | [M + H] = 955.48 | [M + H] = 955.6 |
| 118 | C₅₀H₆₆N₈O₉S | [M + H] = 955.48 | [M + H] = 955.8 |
| 119 | C₅₂H₇₀N₈O₉S | [M + H] = 983.51 | [M + H] = 983.7 |
| 120 | C₄₈H₆₂N₈O₈ | [M + H] = 879.48 | [M + H] = 880.2 |
| 121 | C₅₁H₆₈N₈O₆ | [M + H] = 889.53 | [M + H] = 889.6 |
| 122 | C₅₁H₆₈N₈O₆ | [M + H] = 889.53 | [M + H] = 889.6 |
| 123 | C₅₅H₇₀N₈O₆ | [M + H] = 939.55 | [M + H] = 939.6 |
| 124 | C₅₃H₇₀N₈O₆ | [M + H] = 915.55 | [M + H] = 915.6 |
| 125 | C₅₄H₇₂N₈O₆ | [M + H] = 929.57 | [M + H] = 929.6 |
| 126 | C₅₇H₇₀N₈O₆ | [M + H] = 963.55 | [M + H] = 963.6 |
| 127 | C₅₂H₆₃N₉O₈ | [M + H] = 942.49 | [M + H] = 942.4 |
| 128 | C₅₅H₇₀N₈O₈ | [M + H] = 971.54 | [M + H] = 971.5 |
| 129 | C₄₉H₆₂N₈O₈ | [M + H] = 891.48 | [M + H] = 891.4 |
| 130 | C₄₉H₆₂N₈O₈ | [M + H] = 891.48 | [M + H] = 891.5 |
| 131 | C₅₂H₆₃N₇O₇ | [M + H] = 898.49 | [M + H] = 898.4 |
| 132 | C₅₂H₆₃N₇O₇ | [M + H] = 910.46 | [M + H] = 910.4 |
| 133 | C₅₂H₆₈N₈O₈ | [M + H] = 933.52 | [M + H] = 933.6 |
| 134 | C₅₂H₆₈N₈O₈ | [M + H] = 919.51 | [M + H] = 919.5 |
| 135 | C₅₂H₆₈N₈O₈ | [M + H] = 933.52 | [M + H] = 933.5 |
| 136 | C₅₂H₆₈N₈O₈ | [M + H] = 933.52 | [M + H] = 933.6 |
| 137 | C₅₃H₇₂N₈O₉ | [M + H] = 965.55 | [M + H] = 966.2 |
| 138 | C₅₂H₆₈N₈O₇ | [M + H] = 917.53 | [M + H] = 918.2 |
| 139 | C₅₂H₆₈N₈O₇ | [M + H] = 917.53 | [M + H] = 917.4 |
| 140 | C₄₈H₆₂N₈O₈ | [M + H] = 879.48 | [M + H] = 879.4 |
| 141 | C₅₁H₆₆N₈O₈ | [M + H] = 919.51 | [M + H] = 919.1 |
| 142 | C₅₂H₇₀N₈O₈ | [M + H] = 935.54 | [M + H] = 936.3 |
| 143 | C₅₁H₆₅N₉O₉ | [M + H] = 965.52 | [M + H] = 966.5 |
| 144 | C₅₂H₆₉N₉O₈ | [M + H] = 948.53 | [M + H] = 948.5 |
| 145 | C₅₂H₆₃N₇O₇ | [M + H] = 898.49 | [M + H] = 898.5 |
| 146 | C₅₂H₆₆N₈O₈ | [M + H] = 931.51 | [M + H] = 931.5 |
| 147 | C₄₈H₆₂N₈O₈ | [M + H] = 879.48 | [M + H] = 879.5 |
| 148 | C₅₂H₇₀N₈O₈ | [M + H] = 934.53 | [M + H] = 936.3 |
| 149 | C₅₂H₆₆N₈O₈ | [M + H] = 931.51 | [M + H] = 931.5 |
| 150 | C₄₉H₆₄N₈O₈ | [M + H] = 893.49 | [M + H] = 893.5 |
| 151 | C₅₁H₆₆N₈O₈ | [M + H] = 919.51 | [M + H] = 919.5 |
| 152 | C₅₁H₆₆N₈O₈ | [M + H] = 919.51 | [M + H] = 919.5 |
| 153 | C₅₂H₆₈N₈O₈ | [M + H] = 933.52 | [M + H] = 934.5 |
| 154 | C₅₂H₆₈N₈O₈ | [M + H] = 933.52 | [M + H] = 934.5 |
| 155 | C₅₆H₆₉FN₈O₈ | [M + H] = 1001.53 | [M + H] = 1001.9 |
| 156 | C₅₇H₇₂N₈O₉ | [M + H] = 1013.55 | [M + H] = 1013.9 |
| 157 | C₅₀H₆₄N₈O₈ | [M + H] = 905.49 | [M + H] = 905.5 |
| 158 | C₅₀H₆₄N₈O₈ | [M + H] = 905.49 | [M + H] = 905.6 |
| 159 | C₅₂H₇₀N₈O₉ | [M + H] = 951.53 | [M + H] = 951.6 |
| 160 | C₅₂H₇₀N₈O₉ | [M + H] = 951.53 | [M + H] = 951.6 |
| 161 | C₅₁H₆₈N₈O₉ | [M + H] = 937.52 | [M + H] = 937.6 |
| 162 | C₅₁H₆₈N₈O₉ | [M + H] = 937.52 | [M + H] = 937.6 |
| 163 | C₅₂H₆₆N₈O₈ | [M + H] = 931.51 | [M + H] = 931.5 |
| 164 | C₅₂H₆₆N₈O₈ | [M + H] = 931.51 | [M + H] = 931.5 |
| 165 | C₅₂H₆₈N₈O₈ | [M + H] = 933.53 | [M + H] = 933.5 |
| 166 | C₅₂H₆₆N₈O₈ | [M + H] = 931.51 | [M + H] = 931.5 |
| 167 | C₅₁H₆₆N₈O₈ | [M + H] = 919.51 | [M + H] = 919.7 |
| 168 | C₄₈H₆₄N₁₀O₇S | [M + H] = 925.48 | [M + H] = 925.30 |
| 169 | C₄₈H₆₄N₁₀O₇S | [M + H] = 925.48 | [M + H] = 925.30 |
| 170 | C₅₄H₇₀N₈O₈ | [M + H] = 959.54 | [M + H] = 959.40 |
| 171 | C₅₁H₆₅FN₈O₈ | [M + H] = 937.50 | [M + H] = 937.3 |
| 172 | C₅₁H₆₆N₈O₈ | [M + H] = 919.51 | [M + H] = 919.3 |
| 173 | C₅₂H₆₉N₉O₈ | [M + H] = 948.54 | [M + H] = 948.5 |
| 174 | C₅₄H₆₈N₈O₈ | [M + H] = 957.53 | [M + H] = 957.3 |
| 175 | C₅₄H₆₈N₈O₈ | [M + H] = 957.53 | [M + H] = 957.3 |
| 176 | C₅₂H₆₈N₈O₇ | [M + H] = 917.53 | [M + H] = 918.1 |
| 177 | C₅₁H₆₇N₉O₈ | [M + H] = 934.52 | [M + H] = 934.3 |
| 178 | C₅₁H₆₆N₈O₈ | [M - H] = 917.49 | [M - H] = 917.6 |
| 179 | C₅₁H₆₆N₈O₈ | [M - H] = 917.49 | [M - H] = 917.7 |
| 180 | C₅₃H₆₈N₈O₈ | [M + H] = 945.53 | [M + H] = 945.6 |
| 181 | C₅₃H₆₈N₈O₈ | [M + H] = 943.51 | [M + H] = 943.6 |
| 182 | C₅₁H₆₆N₈O₈ | [M + H] = 919.51 | [M + H] = 919.8 |
| 183 | C₅₁H₆₅N₉O₉ | [M + H] = 918.50 | [M + H] = 948.6 |
| 184 | C₅₁H₆₅FN₈O₈ | [M + H] = 937.50 | [M + H] = 937.3 |
| 185 | C₅₁H₆₅FN₈O₈ | [M + H] = 937.50 | [M + H] = 937.2 |
| 186 | C₅₁H₆₆N₈O₈ | [M + H] = 919.51 | [M + H] = 919.5 |
| 187 | C₅₂H₆₃N₉O₈ | [M + H] = 942.49 | [M + H] = 942.3 |
| 188 | C₅₀H₆₅N₉O₈ | [M + H] = 920.51 | [M + H] = 920.3 |
| 189 | C₅₀H₆₅N₉O₈ | [M + H] = 920.51 | [M + H] = 920.3 |
| 190 | C₅₂H₆₉N₉O₈ | [M + H] = 948.54 | [M + H] = 948.5 |
| 191 | C₅₃H₆₈N₈O₈ | [M + H] = 945.53 | [M + H] = 945.5 |
| 192 | C₅₃H₆₈N₈O₈ | [M + H] = 945.53 | [M + H] = 945.4 |
| 193 | C₅₂H₆₈N₈O₇ | [M + H] = 917.53 | [M + H] = 918.0 |
| 194 | C₅₁H₆₅N₉O₉ | [M + H2O] = 965.50 | [M + H] = 966.4 |
| 195 | C₅₃H₆₈N₈O₈ | [M + H] = 945.53 | [M + H] = 945.5 |
| 196 | C₅₃H₆₈N₈O₈ | [M + H] = 945.53 | [M + H] = 945.4 |
| 197 | C₅₁H₆₇N₉O₈ | [M + H] = 934.52 | [M + H] = 934.5 |
| 198 | C₅₄H₇₀N₈O₈ | [M + H] = 959.54 | [M + H] = 959.3 |
| 199 | C₅₃H₆₈N₈O₈ | [M + H] = 945.53 | [M + H] = 945.4 |
| 200 | C₅₃H₆₈N₈O₈ | [M + H] = 945.53 | [M + H] = 945.5 |
| 201 | C₅₃H₇₂N₈O₉ | [M + H] = 965.55 | [M + H] = 965.4 |
| 202 | C₅₃H₆₈N₈O₈ | [M + H] = 945.53 | [M + H] = 945.8 |
| 203 | C₅₁H₆₇N₉O₈ | [M + H] = 934.52 | [M + H] = 934.8 |
| 204 | C₅₁H₆₉N₉O₈ | [M + H] = 936.54 | [M + H] = 936.8 |
| 205 | C₅₁H₆₅N₉O₉ | [M + H] = 948.50 | [M + H] = 948.5 |
| 206 | C₅₂H₆₈N₈O₈ | [M + H] = 933.53 | [M + H] = 933.7 |
| 207 | C₅₂H₆₈N₈O₈ | [M + H] = 933.53 | [M + H] = 933.5 |
| 208 | C₅₂H₆₈N₈O₈ | [M + H] = 933.53 | [M + H] = 933.7 |
| 209 | C₅₆H₇₆N₁₀O₈ | [M + H] = 1017.59 | [M + H] = 1017.6 |
| 210 | C₅₁H₆₇N₉O₇S | [M + H] = 950.50 | [M + H] = 950.2 |
| 211 | C₅₁H₆₇N₇O₈ | [M + H] = 906.52 | [M + H] = 906.4 |
| 212 | C₅₀H₆₁N₉O₈S | [M + H] = 948.45 | [M + H] = 948.1 |
| 213 | C₄₉H₆₂N₈O₈ | [M + H] = 891.48 | [M + H] = 891.4 |
| 214 | C₅₇H₇₂N₈O₈ | [M + H] = 997.56 | [M + H] = 997.2 |
| 215 | C₅₇H₇₂N₈O₈ | [M + H] = 997.56 | [M + H] = 997.2 |
| 216 | C₄₈H₆₆N₁₀O₇S | [M + H] = 927.49 | [M + H] = 927.5 |
| 217 | C₄₈H₆₆N₁₀O₇S | [M + H] = 927.49 | [M + H] = 927.4 |
| 218 | C₄₈H₆₄N₁₀O₇S | [M + H] = 925.48 | [M + H] = 925.4 |
| 219 | C₄₈H₆₄N₁₀O₇S | [M + H] = 925.48 | [M + H] = 925.1 |
| 220 | C₄₈H₆₄N₁₀O₇S | [M + H] = 925.48 | [M + H] = 925.4 |
| 221 | C₄₈H₆₄N₁₀O₇S | [M + H] = 925.48 | [M + H] = 925.4 |
| 222 | C₄₉H₆₆N₁₀O₇S | [M + H] = 939.49 | [M + H] = 939.5 |
| 223 | C₄₉H₆₆N₁₀O₇S | [M + H] = 939.49 | [M + H] = 939.5 |
| 224 | C₄₉H₆₆N₁₀O₇S | [M + H] = 939.49 | [M + H] = 940.0 |
| 225 | C₄₉H₆₆N₁₀O₇S | [M + H] = 939.49 | [M + H] = 939.2 |
| 226 | C₄₉H₆₆N₁₀O₇S | [M + H] = 939.49 | [M + H] = 939.1 |
| 227 | C₄₉H₆₆N₁₀O₇S | [M + H] = 939.49 | [M + H] = 939.9 |
| 228 | C₄₉H₆₆ N₁₀O₇S | [M + H] = 939.49 | [M + H] = 940.0 |
| 229 | C₄₉H₆₆N₁₀O₇S | [M + H] = 939.49 | [M + H] = 939.1 |
| 230 | C₅₇H₇₇N₉O₈ | [M + H] = 1016.60 | [M + H] = 1016.6 |
| 231 | C₅₁H₆₄N₈O₉ | [M + H] = 933.49 | [M + H] = 933.1 |
| 232 | C₅₁H₆₄N₈O₉ | [M + H] = 933.49 | [M + H] = 933.2 |
| 233 | C₅₅H₆₈F₂N₈O₇ | [M + H] = 991.53 | [M + H] = 992.0 |
| 234 | C₅₄H₆₉N₉O₇S | [M + H] = 988.51 | [M + H] = 988.1 |
| 235 | C₄₉H₆₆N₁₀O₇S | [M + H] = 939.49 | [M + H] = 939.5 |
| 236 | C₅₂H₆₈N₈O₈ | [M + H] = 933.53 | [M + H] = 933.5 |
| 237 | C₅₂H₆₈N₈O₈ | [M + H] = 933.53 | [M + H] = 933.5 |
| 238 | C₅₂H₆₈N₈O₈ | [M + H] = 933.53 | [M + H] = 933.5 |
| 239 | C₅₂H₆₈N₈O₈ | [M + H] = 933.53 | [M + H] = 933.5 |
| 240 | C₅₂H₆₈N₈O₈ | [M + H] = 933.53 | [M + H] = 933.5 |
| 241 | C₅₂H₆₈N₈O₈ | [M + H] = 933.53 | [M + H] = 933.5 |
| 242 | C₅₂H₆₈N₈O₈ | [M + H] = 933.53 | [M + H] = 933.5 |
| 243 | C₅₂H₆₈N₈O₈ | [M + H] = 933.53 | [M + H] = 933.5 |
| 244 | C₅₂H₆₆N₈O₈ | [M + H] = 931.51 | [M + H] = 931.5 |
| 245 | C₅₄H₇₀N₈O₈ | [M + H] = 959.54 | [M + H] = 959.5 |
| 246 | C₅₄H₇₀N₈O₈ | [M + H] = 959.54 | [M + H] = 959.5 |
| 247 | C₅₃H₇₀N₈O₈ | [M + H] = 947.54 | [M + H] = 947.4 |
| 248 | C₅₂H₆₈N₈O₉ | [M + H] = 949.52 | [M + H] = 949.5 |
| 249 | C₅₂H₆₈N₈O₉ | [M + H] = 949.52 | [M + H] = 949.5 |
| 250 | C₅₂H₆₇N₉O₈ | [M + H] = 946.52 | [M + H] = 946.5 |
| 251 | C₅₂H₆₇N₉O₈ | [M + H] = 946.52 | [M + H] = 946.5 |
| 252 | C₅₂H₆₉N₉O₈ | [M + H] = 948.54 | [M + H] = 948.5 |
| 253 | C₅₂H₆₉N₉O₈ | [M + H] = 948.54 | [M + H] = 948.5 |
| 254 | C₅₂H₆₉N₉O₈ | [M + H] = 948.54 | [M + H] = 948.5 |
| 255 | C₅₂H₆₉N₉O₈ | [M + H] = 948.54 | [M + H] = 948.5 |
| 256 | C₅₀H₆₆N₈O₈ | [M + H] = 907.51 | [M + H] = 907.5 |
| 257 | C₅₀H₆₆N₈O₈ | [M + H] = 907.51 | [M + H] = 907.5 |
| 258 | C₅₀H₆₃F₃N₈O₈ | [M + H] = 961.48 | [M + H] = 961.5 |
| 259 | C₅₀H₆₃F₃N₈O₈ | [M + H] = 961.48 | [M + H] = 961.5 |
| 260 | C₅₀H₆₃F₃N₈O₈ | [M + H] = 961.48 | [M + H] = 961.5 |
| 261 | C₅₀H₆₃F₃N₈O₈ | [M + H] = 961.48 | [M + H] = 961.5 |
| 262 | C₅₁H₆₈N₈O₈ | [M + H] = 921.53 | [M + H] = 921.5 |
| 263 | C₅₁H₆₈N₈O₈ | [M + H] = 921.53 | [M + H] = 921.5 |
| 264 | C₅₂H₇₀N₈O₈ | [M + H] = 935.54 | [M + H] = 935.3 |
| 265 | C₅₂H₇₀N₈O₈ | [M + H] = 935.54 | [M + H] = 935.3 |
| 266 | C₅₄H₇₂N₈O₁₀ | [M + H] = 993.55 | [M + H] = 993.5 |
| 267 | C₅₄H₇₂N₈O₁₀ | [M + H] = 993.55 | [M + H] = 993.5 |
| 268 | C₅₆H₇₅N₉O₉ | [M + H] = 1018.58 | [M + H] = 1018.6 |
| 269 | C₅₆H₇₅N₉O₉ | [M + H] = 1018.58 | [M + H] = 1018.5 |
| 270 | C₅₃H₇₁N₉O₉ | [M + H] = 978.55 | [M + H] = 978.5 |
| 271 | C₅₃H₇₁N₉O₉ | [M + H] = 978.55 | [M + H] = 978.5 |
| 272 | C₅₅H₇₃N₉O₁₀ | [M + H] = 1020.56 | [M + H] = 1020.5 |
| 273 | C₅₃H₆₉N₉O₈ | [M + H] = 960.54 | [M + H] = 960.5 |
| 274 | C₅₃H₆₉N₉O₈ | [M + H] = 960.54 | [M + H] = 960.5 |
| 275 | C₅₂H₆₈N₈O₉ | [M + H] = 949.52 | [M + H] = 949.3 |
| 276 | C₅₂H₆₈N₈O₉ | [M + H] = 949.52 | [M + H] = 949.3 |
| 277 | C₅₃H₇₀N₈O₉ | [M + H] = 963.54 | [M + H] = 963.5 |
| 278 | C₅₃H₇₀N₈O₉ | [M + H] = 963.54 | [M + H] = 963.5 |
| 279 | C₅₄H₇₂N₈O₉ | [M + H] = 977.55 | [M + H] = 977.5 |
| 280 | C₅₄H₇₂N₈O₉ | [M + H] = 977.55 | [M + H] = 977.6 |
| 281 | C₅₂H₆₈N₈O₉ | [M + H] = 949.52 | [M + H] = 949.3 |
| 282 | C₅₂H₆₈N₈O₉ | [M + H] = 949.52 | [M + H] = 949.3 |
| 283 | C₅₂H₆₈N₈O₉ | [M + H] = 949.52 | [M + H] = 949.5 |
| 284 | C₅₂H₆₈N₈O₉ | [M + H] = 949.52 | [M + H] = 949.5 |
| 285 | C₅₂H₆₈N₈O₈ | [M + H] = 933.53 | [M + H] = 933.5 |
| 286 | C₄₉H₆₃N₇O₇ | [M + H] = 862.49 | [M + H] = 862.4 |
| 287 | C₅₃H₇₀N₈O₈ | [M + H] = 947.54 | [M + H] = 947.5 |
| 288 | C₅₃H₇₀N₈O₈ | [M + H] = 947.54 | [M + H] = 947.5 |
| 289 | C₅₀H₆₈ N₁₀O₇S | [M + H] = 953.51 | [M + H] = 953.5 |
| 290 | C₅₀H₆₈N₁₀O₇S | [M + H] = 953.51 | [M + H] = 953.4 |
| 291 | C₅₀H₆₈N₁₀O₇S | [M + H] = 953.51 | [M + H] = 953.5 |
| 292 | C₅₀H₆₈N₁₀O₇S | [M + H] = 953.51 | [M + H] = 953.5 |
| 293 | C₄₉H₆₆N₁₀O₇S | [M + H] = 939.49 | [M + H] = 939.5 |
| 294 | C₄₉H₆₆ N₁₀O₇S | [M + H] = 939.49 | [M + H] = 939.5 |
| 295 | C₅₀H₆₈N₁₀O₇S | [M + H] = 953.51 | [M + H] = 953.5 |
| 296 | C₄₉H₆₆N₁₀O₇S | [M + H] = 939.49 | [M + H] = 939.4 |
| 297 | C₄₉H₆₆ N₁₀O₇S | [M + H] = 939.49 | [M + H] = 939.5 |
| 298 | C₄₉H₆₆ N₁₀O₇S | [M + H] = 939.49 | [M + H] = 939.5 |
| 299 | C₄₉H₆₆N₁₀O₇S | [M + H] = 939.49 | [M + H] = 939.5 |
| 300 | C₄₉H₆₆N₁₀O₇S | [M + H] = 939.49 | [M + H] = 939.4 |
| 301 | C₄₉H₆₆N₁₀O₇S | [M + H] = 939.49 | [M + H] = 939.4 |
| 302 | C₄₉H₆₆N₁₀O₇S | [M + H] = 939.49 | [M + H] = 939.4 |
| 303 | C₅₀H₆₆ N₁₀O₇S | [M + H] = 951.49 | [M + H] = 951.5 |
| 304 | C₅₀H₆₆ N₁₀O₇S | [M + H] = 951.49 | [M + H] = 951.5 |
| 305 | C₅₅H₇₀N₈O₈ | [M + H] = 971.54 | [M + H] = 971.1 |
| 306 | C₅₂H₆₆N₈O₈ | [M + H] = 931.51 | [M + H] = 931.2 |
| 307 | C₅₂H₆₆N₈O₈ | [M + H] = 931.51 | [M + H] = 931.2 |
| 308 | C₅₅H₆₉N₉O₈ | [M + H] = 984.54 | [M + H] = 984.5 |
| 309 | C₅₃H₇₀N₈O₈ | [M + H] = 947.54 | [M + H] = 947.5 |
| 310 | C₄₉H₆₆N₁₀O₇S | [M + H] = 939.49 | [M + H] = 939.5 |
| 311 | C₅₁H₆₅FN₈O₈ | [M + H] = 937.50 | [M + H] = 937.2 |
| 312 | C₄₉H₆₃N₇O₈ | [M + H] = 878.48 | [M + H] = 878.4 |
| 313 | C₅₇H₇₂N₈O₇ | [M + H] = 981.56 | [M + H] = 981.5 |
| 314 | C₅₄H₆₉FN₈O₈ | [M + H] = 977.53 | [M + H] = 977.5 |
| 315 | C₅₄H₆₉FN₈O₈ | [M + H] = 977.53 | [M + H] = 977.5 |
| 316 | C₅₅H₆₉N₉O₈ | [M + H] = 984.54 | [M + H] = 984.5 |
| 317 | C₅₈H₇₈N₈O₉ | [M + H] = 1031.60 | [M + H] = 1031.5 |
| 318 | C₅₈H₇₂F₂N₈O₇ | [M + H] = 1031.56 | [M + H] = 1031.5 |
| 319 | C₅₈H₇₂F₂N₈O₇ | [M + H] = 948.48 | [M + H] = 948.4 |
| 320 | C₅₁H₆₅N₉O₇S | [M + H] = 993.54 | [M + H] = 993.5 |
| 321 | C₅₅H₇₀F₂N₈O₇ | [M + H] = 1013.57 | [M + H] = 1013.5 |
| 322 | C₅₅H₇₀F₂N₈O₇ | [M + H] = 989.55 | [M + H] = 989.5 |
| 323 | C₅₈H₇₃FN₈O₇ | [M + H] = 989.55 | [M + H] = 989.5 |
| 324 | C₅₈H₇₃FN₈O₇ | [M + H] = 1086.60 | [M + H] = 1086.6 |
| 325 | C₅₅H₇₂N₈O₉ | [M + H] = 1057.63 | [M + H] = 1057.4 |
| 326 | C₅₅H₇₂N₈O₉ | [M + H] = 967.52 | [M + H] = 967.5 |
| 327 | C₅₉H₇₉N₁₁O₇S | [M + H] = 973.56 | [M + H] = 973.4 |
| 328 | C₅₉H₇₉N₁₁O₇S | [M + H] = 973.56 | [M + H] = 973.5 |
| 329 | C₅₄H₆₉N₇O₁₁ | [M + H] = 992.52 | [M + H] = 992.4 |
| 330 | C₅₁H₆₅N₇O₉ | [M + H] = 920.49 | [M + H] = 920.6 |
| 331 | C₅₇H₆₉N₇O₉ | [M + H] = 996.53 | [M + H] = 996.6 |
| 332 | C₅₂H₆₇N₇O₉ | [M + H] = 934.51 | [M + H] = 934.6 |

### Biological Assays

Compounds 1-2,4-18A, 19A-19B, 21A-24A, 27-32A, 33-43A, 44-45, 47B-54, 56-59, 68A, 69A, 71B, 72A, 73-78, 79B-82A, 83-97, 100-110, 112-117, 119-234, 236-294, and 297-332 exhibited: a) a % cross-linking to KRASG12D of greater than zero within a 24-hour incubation timeframe in the assay described below; and/or b) an IC50 of 2 μM or less in the KRASG12D-B-Raf (AsPC-1) disruption assay described below.

Potency Assay: pERK

The purpose of this assay is to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% paraformaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (PERK, CST-4370, Cell Signaling Technology) was diluted 1:200 in blocking buffer, and 50 μL was added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li—COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

The following compounds exhibited a pERK EC50 of under 5 μM (AsPC-1 KRAS G12D): 179, 157, 178, 327, 205, 106, 242, 121, 183, 36, 158, 196, 84, 17A and B,87, 187, 114, 182, 255, 254, 185, 236, 124, 197, 1, 107, 192, 34, 118, 296, 78, 89, 104, 74, 306, 310, 105, 152, 269, 229, 221, 294, 117, 119, 240, 151, 1 93, 86, 245, 128, 163, 272, 270, 79A and B, 232, 140, 138, 293, 38, 94, 110, 172, 271, 246, 72A and B, 108, 35, 1 4, 127, 7, 153, 39, 190, 96, 227, 13, 77, 286, 215, 244, 184, 284, 275, 147, 295, 204, 50, 161, 129, 176, 51, 290, 2 26, 218, 164, 282, 167, 162, 131, 228, 292, 233, 308, 304, 48, 9, 113, 298, 277, 54, 57, 219, 173, 220, 268, 49, 14 9, 247, 120, 154, 307, 56, 166, 11, 53, 101, 10, 8, 238, 97, 303, 132, 186, 52, 297, 93, 85, 83, 280, 103, 200, 276, 2 78, 144, 165, 199, 33, 139, 112, 224, 177, 241, 273, 237, 274, 191, 243, 319, 320, 225, 59, 311, 207, 239, 279, 1 60, 289, 171, 156, 92, 202, 43A, 266, 208, 281, 159, 300, 210, 223, 217, 283, 216, 231, 299, 90, 91, 267, 155, 25 9, 291, 258, 257, 262, 222, 137, 100, 256, 88, 316, 142, 318, 146, 198, 288, 302, 174, 265, 322, 12, 168, 42A, 20 1, 301 , 263, 248, 287, 58, 305, 260, 134, 169, 313, 314, 323, 234, 136, 148, 102, 315, 141, 150, 309, 326, 261, 3 21, 175, 230, 249, 264, 95, 285, 135, 133, 170, 317, 328, 214, 209, 324, 325.

### Determination of Cell Viability in RAS Mutant Cancer Cell Lines Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 μL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

### *Key:

+++++: IC50≥10 μM
++++10 μM>IC50≥1 μM+
+++1 μM>IC50≥0.1 μM+
++: 0.1 μM>IC50≥0.01 μM
+: IC50<0.01 μM

TABLE 5
H358 Cell Viability assay data (K-Ras G12C, IC50, uM):

| IC50* | Examples of A compounds |
|---|---|
| + | 1, 44, 58, 59, 90, 95, 106, 136, 137, 142, 146, 148, 150, |
| | 155, 156, 159, 160, 165, 171, 174, 175, 186, 201, 207, |
| | 209, 222, 231, 236, 241, 261, 266, 267, 273, 274, 279, |
| | 280, 299, 301, 307, 319, 324, 325, 328, 42 A, 43 A |
| ++ | 2, 5, 7, 8, 9, 10, 13, 33, 41, 83, 85, 100, 102, 132, 133, |
| | 135, 144, 168, 169, 170, 191, 208, 214, 216, 217, 223, |
| | 224, 225, 226, 227, 228, 230, 234, 239, 248, 249, 256, |
| | 257, 258, 259, 260, 262, 263, 264, 265, 270, 276, 277, |
| | 283, 287, 288, 289, 290, 291, 292, 293, 296, 297, 298, |
| | 300, 302, 303, 316, 317, 322, 323, 326, 42 B, 43 B |
| +++ | 3, 4, 6, 11, 12, 14, 40, 45, 46, 48, 50, 52, 56, 63, 77, 103, |
| | 107, 141, 202, 210, 243, 285, 294, 304, 313, 318, 320, 321 |
| ++++ | 26, 29, 30, 31, 49, 60, 66, 96, 15 A and B, 16 A and B, 16 |
| | A and B, 17 A and B, 17 A and B, 18 A and B, 18 A and |
| | B, 19 A and B, 19 A and B, 20 A and B, 20 A and B, 22 A |
| | and B, 23 Aand B, 24 A and B, 24 A and B, 32 A and B, |
| | 32 A and B, 47 A and B, 62 A and B, 62 A and B, 68 A |
| | and B, 68 A and B, 69 A and B, 69 A and B |
| +++++ | 15 A and B, 25, 27, 28, 47 A and B, 67 |

TABLE 6
AsPC-1 Cell Viability assay data (K-Ras G12D, IC50, uM):

| IC50* | Examples of A compounds |
|---|---|
| + | 209, 325 |
| ++ | 95, 133, 170, 175, 214, 230, 249, 285, 309, 313, 317, 321, |
| | 324 |
| +++ | 7, 12, 58, 59, 77, 88, 90, 100, 102, 103, 135, 136, 137, 141, |
| | 142, 144, 146, 148, 150, 155, 156, 159, 160, 168, 169, 171, |
| | 174, 186, 198, 201, 207, 210, 216, 217, 222, 223, 224, 225, |
| | 228, 231, 234, 239, 241, 243, 248, 256, 257, 258, 259, 260, |
| | 261, 262, 263, 264, 265, 266, 267, 270, 273, 274, 277, 279, |
| | 281, 283, 287, 288, 289, 291, 297, 298, 299, 300, 301, 302, |
| | 303, 305, 307, 311, 314, 315, 318, 319, 320, 323, 326, 328, |
| | 19 A and B, 42 A, 43 A |
| ++++ | 1, 8, 9, 10, 11, 13, 14, 29, 34, 35, 38, 39, 44, 48, 49, 52, 53, |
| | 54, 56, 57, 60, 63, 73, 74, 75, 76, 78, 80, 81, 83, 84, 85, 86, |
| | 87, 91, 92, 96, 97, 101, 104, 105, 106, 107, 108, 110, 112, |
| | 113, 114, 117, 119, 120, 121, 122, 128, 129, 132, 134, 139, |
| | 140, 147, 149, 151, 152, 153, 154, 157, 158, 161, 162, 163, |
| | 164, 165, 166, 167, 172, 173, 176, 177, 184, 185, 187, 190, |
| | 191, 192, 193, 196, 197, 199, 200, 202, 204, 208, 215, 218, |
| | 219, 220, 221, 226, 227, 229, 232, 233, 236, 237, 238, 240, |
| | 2242, 244, 245, 246, 247, 255, 268, 69, 272, 275, 276, 278, |
| | 280, 282, 284, 286, 290, 292, 293, 294, 295, 296, 304, 306, |
| | 308, 310, 316, 322, 327, 16 A and B, 16 A and B, 17 A and |
| | B, 17 A and B, 18 A and B, 18 A and B, 22 A and B, 23 A |
| | and B, 32 A and B, 42 B, 61 A and B, 61 A and B, 62 A and |
| | B, 62 A and B, 64 A and B, 65 A and B, 65 A and B, 68 A |
| | and B, 69 A and B, 69 A and B, 70 A and B, 71 A and B, |
| | 72 A and B, 72 A and B, 79 A and B, 79 A and B, 82 A and |
| | B, 82 A and B |
| +++++ | 2, 3, 4, 5, 6, 28, 31, 36, 37, 40, 41, 45, 46, 50, 51, 55, 66, 67, |
| | 89, 93, 94, 98, 99, 109, 111, 115, 116, 118, 126, 127, 130, 1 |
| | 31, 138, 143, 145, 178, 179, 180, 181, 182, 183, 188, 189, |
| | 194, 195, 203, 205, 206, 211, 212, 213, 235, 250, 251, 252, |
| | 253, 254, 271, 312, 15 A and B, 15 A and B, 20 A and B, |
| | 20 A and B, 24 A and B, 24 A and B, 32 A and B, 43 B, |
| | 47 A and B, 47 A and B, 64 A and B, 70 A and B, 71 A and B |

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides, such as K-Ras G12D and K-Ras G13D.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl2, tagless Cyclophilin A, His6-K-Ras-GMPPNP, and GST-BRAF^{RBD} were combined in a 384-well assay plate at final concentrations of 25 μM, 12.5 nM, and 50 nM, respectively. Compound was present in plate wells as a 10-point 3-fold dilution series starting at a final concentration of 30 μM. After incubation at 25° C. for 3 hours, a mixture of anti-His Eu—W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras: RAF complex were identified as those eliciting a decrease in the TR-FRET ratio relative to DMSO control wells.

### Ras-Raf Disruption/FRET/MOA Assay Data (IC50, μM):

### *Key:

+++++: IC50≥10 μM
++++10 μM>IC50≥1 μM+
+++1 μM>IC50≥0.1 μM+
++: 0.1 μM>IC50≥0.01 μM
+: IC50<0.01 μM

TABLE 7
KRAS G13D FRET data

| IC50* | Examples of A compounds |
|---|---|
| + | None |
| ++ | 12, 58, 83, 85, 86, 88, 89, 95, 100, 109, 113, 115, 120, 129, |
| | 130, 133, 135, 136, 137, 138, 139, 140, 143, 148, 149, 150, |
| | 157, 158, 162, 167, 170, 171, 172, 173, 174, 175, 176, 177, |
| | 182, 186, 193, 194, 195, 196, 197, 198, 204, 209, 230, 231, |
| | 239, 241, 248, 249, 256, 257, 262, 263, 264, 265, 274, 276, |
| | 285, 286, 288, 306, 307, 309, 325, 329, 330, 332, 258*, 259*, |
| | 260*, 261*, 281*, 282*, 283*, 284*, 314*, 315*, 316*, 42 A, |
| | 43 A |
| +++ | 1, 2, 3, 4, 5, 7, 8, 9, 11, 13, 14, 33, 34, 35, 36, 37, 38, 39, 40, |
| | 41, 44, 45, 48, 49, 50, 51, 52, 53, 54, 56, 57, 59, 84, 87, 90, 91, |
| | 92, 93, 94, 96, 97, 101, 102, 103, 104, 105, 108, 110, 111, 112, |
| | 116, 117, 118, 119, 127, 128, 131, 134, 141, 142, 144, 145, |
| | 146, 147, 151, 152, 153, 155, 156, 159, 160, 161, 163, 164, |
| | 165, 166, 168, 169, 178, 179, 180, 181, 183, 184, 185, 187, |
| | 188, 189, 190, 191, 199, 200, 201, 202, 203, 205, 206, 207, |
| | 208, 212, 213, 214, 215, 216, 217, 218, 219, 220, 221, 222, |
| | 223, 224, 225, 226, 227, 228, 229, 232, 236, 237, 238, 242, |
| | 243, 244, 245, 246, 247, 252, 253, 254, 255, 266, 267, 268, |
| | 269, 270, 271, 272, 273, 275, 277, 278, 279, 280, 287, 289, |
| | 291, 293, 294, 297, 298, 299, 300, 301, 302, 303, 305, 308, |
| | 311, 317, 322, 323, 326, 328, 331, 47 A and B, 69 A and B |
| ++++ | 6, 10, 25, 26, 27, 28, 29, 30, 31, 46, 55, 66, 67, 73, 74, 98, 106, |
| | 107, 114, 126, 132, 154, 192, 210, 211, 233, 235, 240, 250, |
| | 251, 290, 292, 295, 296, 304, 310, 312, 313, 318, 319, 320, |
| | 321, 324, 15 A and B, 17 A and B, 18 A and B, 19 A and B, |
| | 24 A and B, 32 A and B, 32 A and B, 42 B, 43 B, 47 A and B, |
| | 62 A and B, 72 A and B |
| +++++ | 60, 63, 75, 76, 77, 78, 80, 81, 99, 121, 122, 123, 124, 125, 234, |
| | 327, 15 A and B, 16 A and B, 16 A and B, 17 A and B, 18 A |
| | and B, 19 A and B, 20 A and B, 20 A and B, 22 A and B, 23 A |
| | and B, 24 A and B, 61 A and B, 61 A and B, 62 A and B, 64 A |
| | and B, 64 A and B, 65 A and B, 65 A and B, 68 A and B, 68 A |
| | and B, 69 A and B, 70 A and B, 70 A and B, 71 A and B, 71 A |
| | and B, 72 A and B, 79 A and B, 79 A and B, 82 A and B, 82 A |
| | and B |

TABLE 8
KRAS G12S FRET data

| IC50* | Examples of A compounds |
|---|---|
| + | 325, 264, 281, 135 |
| ++ | 263, 258, 230, 249, 287, 282, 209, 139, 315, 170, 283, 267, |
| | 150, 260, 274, 284, 42 A, 167, 276, 261, 133, 137, 12, 256, |
| | 136, 257, 285, 288, 265, 88, 85, 231, 316, 330, 269, 262, 280, |
| | 148, 186, 278, 182, 248, 259, 309, 162, 317, 314, 332, 160, |
| | 140, 175, 305, 95, 308, 158, 113, 273, 323, 268, 173, 322, 306, |
| | 43 A, 214, 177, 171, 161, 146, 272, 207, 329, 157, 197, 201, |
| | 147, 103, 208, 200, 328, 241, 172, 90, 89, 105, 149, 279, 100, |
| | 159, 238, 93, 271, 91, 48, 174, 7, 35, 155, 307, 53, 36, 52, 92, |
| | 190, 156, 237, 302, 326, 59, 8, 142, 37, 33, 11, 141, 97, 127, |
| | 10, 13, 277, 58, 104, 275, 222, 129, 266, 204, 39, 191, 110, |
| | 176, 301, 96, 331, 54, 198, 239, 130, 255, 134, 83, 50, 1, 34, |
| | 289, 86, 38, 47 A and B, 168, 143, 169, 270, 298 |
| +++ | 14, 115, 152, 109, 49, 102, 223, 189, 44, 291, 144, 297, 120, |
| | 202, 153, 187, 185, 188, 56, 138, 195, 224, 254, 51, 178, 244, |
| | 286, 194, 45, 225, 181, 246, 9, 205, 166, 101, 196, 252, 2, 116, |
| | 243, 193, 216, 253, 179, 293, 217, 192, 112, 245, 311, 199, |
| | 203, 94, 165, 3, 163, 232, 151, 5, 294, 108, 183, 46, 215, 299, |
| | 119, 117, 4, 300, 164, 303, 184, 213, 227, 145, 57, 128, 304, 6, |
| | 220, 229, 218, 226, 228, 87, 251, 131, 84, 219, 111, 221, 242, |
| | 154, 310, 247, 180, 40, 47 A and B, 41, 290, 118, 236, 292, 31, |
| | 324, 296, 321, 250, 212, 206, 55, 126 |
| ++++ | 210, 240, 77, 121, 211, 319, 42 B, 313, 132, 114, 98, 320, 295, |
| | 318, 43 B, 235, 122, 107, 62 A and B, 312, 24 A and B, 106, |
| | 74, 234, 78, 66, 15 A and B, 233, 73, 69 A and B, 63, 29, 67, |
| | 22 A and B, 80, 23 A and B |
| +++++ | 25, 124, 32 A and B, 327, 27, 28, 26, 62 A and B, 72 A and B, |
| | 20 A and B, 79 A and B, 19 A and B, 16 A and B, 123, 24 A |
| | and B, 30, 17 A and B, 15 A and B, 19 A and B, 32 A and B, |
| | 18 A and B, 18 A and B, 17 A and B, 16 A and B, 20 A and B, |
| | 69 A and B, 60, 68 A and B, 68 A and B, 65 A and B, 65 A and |
| | B, 64 A and B, 64 A and B, 70 A and B, 71 A and B, 70 A and |
| | B, 71 A and B, 72 A and B, 61 A and B, 61 A and B, 82 A and |
| | B, 82 A and B, 81, 79 A and B, 76, 75, 99, 125 |

TABLE 9
KRAS G13C FRET data

| IC50* | Examples of A compounds |
|---|---|
| + | 84, 85, 89, 129, 135, 136, 139, 140, 182, 195, 205, 209, 230, |
| | 231, 256, 257, 258, 260, 282, 283, 284, 285, 325, 329, 330, |
| | 42 A |
| ++ | 3, 4, 7, 8, 11, 12, 13, 14, 34, 35, 36, 37, 38, 39, 41, 44, 45, 48, |
| | 49, 50, 51, 53, 54, 56, 57, 58, 59, 77, 78, 83, 86, 87, 88, 90, 91, |
| | 92, 93, 94, 95, 96, 97, 100, 102, 103, 104, 105, 108, 109, 112, |
| | 113, 115, 117, 118, 119, 120, 124, 127, 128, 130, 131, 133, |
| | 134, 137, 138, 141, 142, 143, 144, 145, 146, 147, 148, 149, |
| | 150, 152, 155, 156, 157, 158, 159, 160, 161, 162, 167, 169, |
| | 170, 171, 172, 173, 174, 175, 176, 177, 178, 179, 180, 181, |
| | 183, 184, 185, 186, 187, 188, 189, 190, 191, 193, 194, 196, |
| | 197, 198, 200, 201, 203, 204, 207, 208, 212, 213, 214, 215, |
| | 232, 237, 238, 239, 241, 245, 246, 248, 249, 251, 252, 253, |
| | 254, 255, 259, 261, 262, 263, 264, 265, 266, 267, 268, 269, |
| | 270, 271, 272, 273, 274, 275, 276, 277, 278, 279, 280, 281, |
| | 286, 287, 288, 297, 301, 305, 306, 307, 308, 309, 311, 314, |
| | 315, 316, 317, 322, 323, 326, 328, 331, 332, 43 A, 47 A and |
| | B, 47 A and B, 69 A and B |
| +++ | 1, 2, 5, 6, 9, 10, 31, 33, 40, 46, 52, 55, 66, 74, 76, 98, 101, |
| | 106, 107, 110, 111, 114, 116, 121, 122, 123, 125, 126, 151, |
| | 153, 154, 163, 164, 165, 166, 168, 192, 199, 202, 206, 211, |
| | 216, 217, 218, 219, 220, 221, 222, 223, 224, 225, 226, 227, |
| | 228, 229, 233, 236, 243, 244, 247, 250, 289, 290, 291, 292, |
| | 293, 294, 296, 298, 299, 300, 302, 303, 310, 312, 313, 318, |
| | 319, 320, 321, 324, 22 A and B, 42 B, 70 A and B |
| ++++ | 25, 27, 28, 29, 63, 67, 73, 80, 132, 210, 234, 235, 240, 242, |
| | 295, 304, 327, 15 A and B, 16 A and B, 17 A and B, 17 A and |
| | B, 18 A and B, 19 A and B, 20 A and B, 23 A and B, 24 A |
| | and B, 32 A and B, 43 B, 62 A and B, 65 A and B, 68 A and |
| | B, 69 A and B, 72 A and B, 79 A and B |
| +++++ | 26, 30, 60, 75, 81, 99, 15 A and B, 16 A and B, 18 A and B, |
| | 19 A and B, 20 A and B, 24 A and B, 32 A and B, 61 A and |
| | B, 61 A and B, 62 A and B, 64 A and B, 64 A and B, 65 A |
| | and B, 68 A and B, 70 A and B, 71 A and B, 71 A and B, 72 |
| | A and B, 79 A and B, 82 A and B, 82 A and B |

TABLE 10
KRAS G12V FRET data

| IC50* | Examples of A compounds |
|---|---|
| + | 325 |
| ++ | 1, 11, 12, 13, 36, 44, 48, 50, 58, 59, 83, 85, 88, 90, 93, 95, 96, 97, |
| | 100, 103, 113, 133, 135, 136, 137, 139, 140, 141, 146, 147, 148, |
| | 149, 150, 151, 155, 156, 157, 158, 159, 160, 161, 162, 165, 167, |
| | 170, 171, 172, 173, 174, 175, 177, 182, 186, 189, 190, 192, 197, |
| | 200, 201, 204, 207, 208, 209, 214, 230, 231, 239, 241, 248, 249, |
| | 256, 257, 258, 259, 260, 261, 262, 263, 264, 265, 267, 268, 269, |
| | 272, 273, 274, 276, 278, 279, 280, 281, 282, 283, 284, 285, 287, |
| | 288, 302, 305, 306, 307, 309, 314, 315, 316, 317, 322, 323, 326, |
| | 328, 329, 330, 331, 332, 42 A, 43 A |
| +++ | 2, 3, 4, 5, 6, 7, 8, 9, 10, 14, 31, 33, 34, 35, 37, 38, 39, 40, 41, 45, |
| | 49, 51, 52, 53, 54, 55, 56, 57, 84, 86, 87, 89, 91, 92, 94, 101, 102, |
| | 104, 105, 108, 109, 110, 111, 112, 115, 116, 117, 118, 119, 120, |
| | 127, 128, 129, 130, 131, 134, 142, 143, 144, 145, 152, 153, 154, |
| | 163, 164, 166, 168, 169, 176, 178, 179, 180, 181, 183, 184, 185, |
| | 187, 188, 191, 194, 195, 196, 198, 199, 202, 203, 205, 206, 212, |
| | 213, 215, 216, 217, 218, 219, 220, 221, 222, 223, 224, 225, 226, |
| | 227, 228, 229, 232, 236, 237, 238, 243, 244, 245, 246, 247, 250, |
| | 251, 252, 253, 254, 255, 266, 270, 271, 275, 277, 286, 289, 290, |
| | 291, 292, 293, 294, 297, 298, 299, 300, 301, 303, 304, 308, 310, |
| | 311, 321, 324, 47 A and B |
| ++++ | 29, 46, 66, 67, 74, 77, 78, 80, 98, 106, 107, 114, 121, 122, 123, |
| | 124, 126, 132, 138, 193, 210, 211, 233, 234, 235, 240, 242, 295, |
| | 296, 312, 313, 318, 319, 320, 15 A and B, 16 A and B, 22 A and |
| | B, 24 A and B, 42 B, 43 B, 47 A and B, 62 A and B, 69 A and B, |
| | 72 A and B, 79 A and B |
| ++++ | 73, 25, 26, 27, 28, 30, 60, 63, 75, 76, 81, 99, 125, 327, 15 A and |
| | B, 16 A and B, 17 A and B, 17 A and B, 18 A and B, 18 A and B, |
| | 19 A and B, 19 A and B, 20 A and B, 20 A and B, 23 A and B, 24 |
| | A and B, 32 A and B, 32 A and B, 61 A and B, 61 A and B, 62 A |
| | and B, 64 A and B, 64 A and B, 65 A and B, 65 A and B, 68 A |
| | and B, 68 A and B, 69 A and B, 70 A and B, 70 A and B, 71 A |
| | and B, 71 A and B, 72 A and B, 79 A and B, 82 A and B, 82 A |
| | and B |

TABLE 11
KRAS G12D FRET data

| IC50* | Examples of A compounds |
|---|---|
| + | 214, 305, 325 |
| ++ | 59, 88, 95, 103, 113, 133, 135, 136, 137, 139, 140, 141, 146, |
| | 148, 150, 167, 170, 171, 173, 175, 186, 198, 209, 230, 231, 241, |
| | 248, 249, 256, 258, 260, 261, 263, 264, 265, 267, 269, 274, 276, |
| | 278, 280, 281, 282, 283, 284, 285, 287, 309, 315, 316, 330, 42 |
| | A, 43 A |
| +++ | 1, 2, 7, 8, 12, 13, 14, 33, 34, 35, 36, 37, 38, 39, 40, 41, 44, 45, |
| | 48, 49, 50, 51, 52, 53, 54, 56, 58, 73, 74, 83, 85, 86, 89, 90, 91, |
| | 92, 93, 96, 97, 100, 101, 102, 104, 105, 110, 112, 115, 120, 127, |
| | 128, 129, 130, 131, 134, 138, 142, 143, 144, 147, 149, 151, 152, |
| | 153, 155, 156, 157, 158, 159, 160, 161, 162, 163, 164, 165, 166, |
| | 168, 169, 172, 174, 176, 177, 178, 179, 181, 182, 184, 185, 187, |
| | 188, 189, 190, 191, 192, 193, 194, 196, 197, 199, 200, 201, 202, |
| | 203, 204, 207, 208, 215, 216, 217, 222, 223, 224, 225, 227, 236, |
| | 237, 238, 239, 242, 243, 244, 245, 252, 253, 254, 255, 257, 259, |
| | 262, 266, 268, 270, 271, 272, 273, 275, 277, 279, 286, 288, 289, |
| | 291, 293, 294, 297, 298, 299, 300, 301, 302, 303, 304, 306, 307, |
| | 308, 311, 313, 314, 317, 321, 322, 323, 324, 326, 328, 329, 331, |
| | 332, 70 A and B |
| ++++ | 16 A and B, 3, 4, 5, 6, 9, 10, 11, 29, 31, 46, 57, 63, 66, 67, 76, |
| | 77, 78, 80, 84, 87, 94, 98, 106, 107, 108, 109, 111, 114, 116, |
| | 117, 118, 119, 121, 122, 123, 124, 125, 126, 132, 145, 154, 180, |
| | 183, 195, 205, 206, 210, 211, 212, 213, 218, 219, 220, 221, 226, |
| | 228, 229, 232, 233, 234, 235, 240, 246, 247, 250, 251, 290, 292, |
| | 295, 296, 310, 312, 318, 319, 320, 327, 15 A and B, 17 A and B, |
| | 17 A and B, 18 A and B, 19 A and B, 22 A and B, 23 A and B, |
| | 24 A and B, 32 A and B, 42 B, 43 B, 47 A and B, 47 A and B, |
| | 62 A and B, 68 A and B, 69 A and B, 72 A and B, 82 A and B |
| +++++ | 25, 26, 27, 28, 30, 55, 60, 75, 81, 99, 15 A and B, 16 A and B, |
| | 18 A and B, 19 A and B, 20 A and B, 20 A and B, 24 A and B, |
| | 32 A and B, 61 A and B, 61 A and B, 62 A and B, 64 A and B, |
| | 64 A and B, 65 A and B, 65 A and B, 68 A and B, 69 A and B, |
| | 70 A and B, 71 A and B, 71 A and B, 72 A and B, 79 A and B, |
| | 79 A and B, 82 A and B |

TABLE 12
KRAS WT FRET data

| IC50* | Examples of A compounds |
|---|---|
| + | 264, 325 |
| ++ | 12, 13, 35, 36, 37, 44, 45, 52, 53, 54, 58, 59, 83, 85, 86, 88, 89, |
| | 90, 91, 92, 93, 95, 96, 97, 100, 102, 103, 105, 109, 110, 112, |
| | 113, 115, 116, 120, 129, 130, 133, 134, 135, 136, 137, 138, 139, |
| | 140, 141, 143, 146, 147, 148, 149, 150, 155, 156, 157, 158, 159, |
| | 160, 161, 162, 167, 168, 169, 170, 171, 172, 173, 174, 175, 176, |
| | 177, 182, 183, 186, 189, 190, 191, 193, 195, 196, 197, 198, 201, |
| | 204, 205, 207, 208, 209, 213, 214, 222, 223, 230, 231, 237, 238, |
| | 239, 241, 245, 246, 248, 249, 252, 253, 256, 257, 258, 259, 260, |
| | 261, 262, 263, 265, 267, 268, 269, 271, 272, 273, 274, 276, 278, |
| | 279, 280, 281, 282, 283, 284, 285, 287, 288, 289, 291, 297, 298, |
| | 301, 302, 305, 306, 307, 308, 309, 314, 315, 316, 317, 322, 323, |
| | 326, 328, 329, 330, 331, 332, 42A, 43A |
| +++ | 1, 2, 3, 4, 5, 6, 7, 8, 9, 10, 11, 14, 33, 34, 38, 39, 40, 41, 48, 49, |
| | 50, 51, 56, 57, 84, 87, 94, 98, 101, 104, 108, 111, 117, 118, 119, |
| | 121, 127, 128, 131, 142, 144, 145, 151, 152, 153, 154, 163, 164, |
| | 165, 166, 178, 179, 180, 181, 184, 185, 187, 188, 192, 194, 199, |
| | 200, 202, 203, 206, 211, 212, 215, 216, 217, 218, 219, 220, 221, |
| | 224, 225, 226, 227, 228, 229, 232, 236, 240, 242, 243, 244, 247, |
| | 250, 251, 254, 255, 266, 270, 275, 277, 286, 290, 292, 293, 294, |
| | 295, 296, 299, 300, 303, 304, 310, 311, 312, 318, 321, 324, 47 |
| | A and B |
| ++++ | 25, 26, 27, 29, 30, 31, 46, 55, 63, 66, 67, 73, 74, 76, 77, 78, 80, |
| | 106, 107, 114, 122, 123, 124, 126, 132, 210, 233, 234, 235, 313, |
| | 319, 320, 327, 15 A and B, 16 A and B, 22 A and B, 23 A and |
| | B, 24 A and B, 42B, 43B, 47 A and B, 62 A and B, 69 A and B, |
| | 79 A and B |
| +++++ | 28, 60, 75, 81, 99, 125, 15 A and B, 16 A and B, 17 A and B, |
| | 17 A and B, 18 A and B, 18 A and B, 19 A and B, 19 A and B, |
| | 20 A and B, 20 A and B, 24 A and B, 32 A and B, 32 A and B, |
| | 61 A and B, 61 A and B, 62 A and B, 64 A and B, 64 A and B, |
| | 65 A and B, 65 A and B, 68 A and B, 68 A and B, 69 A and B, |
| | 70 A and B, 70 A and B, 71 A and B, 71 A and B, 72 A and B, |
| | 72 A and B, 79 A and B, 82 A and B, 82 A and B |

TABLE 13
KRAS G12C FRET data

| IC50* | Examples of A compounds |
|---|---|
| + | 1, 7, 41, 58, 88, 90, 95, 109, 113, 115, 135, 137, 139, 140, 142, |
| | 146, 148, 149, 150, 151, 156, 161, 162, 165, 167, 171, 174, 175, |
| | 183, 184, 186, 190, 192, 196, 198, 200, 201, 203, 204, 209, 230, |
| | 231, 241, 249, 252, 263, 264, 266, 267, 268, 269, 270, 271, 272, |
| | 273, 274, 280, 281, 306, 307, 309, 311, 312, 325, 329, 330, 331, |
| | 42 A, 43 A |
| ++ | 2, 3, 4, 5, 6, 8, 9, 10, 11, 12, 13, 14, 31, 33, 34, 35, 36, 37, 38, 39, |
| | 44, 45, 48, 49, 50, 51, 52, 53, 54, 56, 59, 73, 74, 77, 78, 83, 84, |
| | 85, 86, 87, 89, 91, 92, 93, 94, 96, 97, 100, 101, 102, 103, 104, |
| | 105, 106, 110, 114, 117, 119, 124, 125, 128, 129, 130, 131, 133, |
| | 134, 136, 138, 141, 143, 144, 147, 152, 153, 154, 155, 157, 158, |
| | 159, 160, 168, 169, 170, 172, 173, 176, 177, 178, 179, 181, 182, |
| | 185, 188, 189, 191, 193, 195, 197, 202, 205, 206, 207, 208, 211, |
| | 213, 214, 215, 216, 217, 220, 222, 223, 224, 225, 232, 236, 237, |
| | 238, 239, 243, 244, 245, 246, 247, 248, 250, 251, 253, 254, 255, |
| | 256, 257, 258, 259, 260, 261, 262, 265, 275, 276, 277, 278, 279, |
| | 282, 283, 284, 285, 287, 288, 289, 291, 293, 294, 297, 298, 299, |
| | 301, 302, 305, 308, 314, 315, 316, 317, 319, 322, 323, 326, 328, |
| | 332, 16 A and B, 17 A and B, 18 A and B, 22 A and B, 23 A and |
| | B, 32 A and B, 43 B, 68 A and B, 69 A and B, 82 A and B |
| +++ | 40, 46, 55, 57, 66, 67, 75, 76, 80, 81, 98, 107, 108, 111, 112, 116, |
| | 118, 120, 121, 122, 123, 126, 127, 132, 145, 163, 164, 166, 180, |
| | 187, 194, 199, 210, 212, 218, 219, 221, 226, 227, 228, 229, 233, |
| | 234, 235, 240, 242, 286, 290, 292, 295, 296, 300, 303, 304, 310, |
| | 313, 318, 320, 321, 324, 15 A and B, 16 A and B, 17 A and B, 19 |
| | A and B, 20 A and B, 24 A and B, 42 B, 47 A and B, 61 A and |
| | B, 62 A and B, 70 A and B, 72 A and B, 79 A and B |
| ++++ | 25, 26, 27, 28, 29, 63, 327, 18 A and B, 32 A and B, 47 A and B, |
| | 62 A and B, 65 A and B, 68 A and B, 69 A and B, 71 A and B, 72 |
| | A and B, 82 A and B |
| ++++ | 30, 60, 99, 15 A and B, 19 A and B, 20 A and B, 24 A and B, 61 |
| | A and B, 64 A and B, 64 A and B, 65 A and B, 70 A and B, 71 A |
| | and B, 79 A and B |

TABLE 14
KRAS Q61H FRET data

| IC50* | Examples of A compounds |
|---|---|
| + | 209, 230, 231, 246, 249, 258, 264, 281, 325 |
| ++ | 36, 37, 91, 92, 108, 117, 119, 141, 144, 163, 164, 166, 167, 170, |
| | 186, 202, 203, 204, 205, 207, 208, 212, 213, 214, 215, 216, 217, |
| | 218, 220, 222, 223, 224, 225, 227, 228, 229, 232, 237, 238, 239, |
| | 241, 244, 245, 248, 252, 253, 254, 255, 256, 257, 259, 260, 261, |
| | 262, 263, 265, 266, 267, 268, 269, 271, 272, 273, 274, 275, 276, |
| | 277, 278, 279, 280, 282, 283, 284, 285, 286, 287, 288, 289, 291, |
| | 293, 294, 297, 298, 299, 300, 301, 302, 303, 304, 305, 306, 307, |
| | 308, 309, 311, 314, 315, 316, 317, 322, 323, 326, 328, 329, 330, |
| | 331, 332 |
| +++ | 165, 206, 210, 211, 219, 221, 226, 233, 235, 236, 240, 242, 243, |
| | 247, 250, 251, 270, 290, 292, 295, 296, 310, 312, 313, 318, 319, |
| | 320, 321, 324 |
| ++++ | 234, 327 |
| +++++ | None |

TABLE 15
NRAS G12C FRET data

| IC50* | Examples of A compounds |
|---|---|
| + | 1, 5, 7, 41, 58, 59, 86, 88, 90, 95, 109, 113, 115, 135, 136, |
| | 137, 138, 139, 140, 142, 146, 148, 149, 150, 151, 155, 156, |
| | 159, 160, 161, 162, 165, 167, 171, 174, 175, 184, 186, 192, |
| | 196, 198, 200, 201, 203, 204, 209, 230, 231, 241, 249, 252, |
| | 263, 264, 266, 267, 268, 269, 270, 271, 272, 273, 274, 276, |
| | 278, 280, 281, 284, 306, 307, 309, 311, 312, 325, 329, 330, |
| | 42 A, 43 A |
| ++ | 2, 3, 6, 8, 9, 10, 11, 12, 13, 14, 33, 34, 35, 36, 37, 38, 39, |
| | 44, 45, 48, 49, 50, 52, 53, 54, 73, 74, 77, 78, 83, 84, 85, 87, |
| | 89, 91, 92, 93, 96, 97, 100, 101, 102, 103, 104, 105, 106, |
| | 110, 114, 124, 125, 128, 129, 130, 131, 133, 134, 141, 143, |
| | 144, 147, 152, 153, 154, 157, 158, 168, 169, 170, 172, 173, |
| | 176, 177, 178, 179, 181, 182, 183, 185, 188, 189, 190, 191, |
| | 193, 195, 197, 202, 205, 206, 207, 208, 211, 213, 214, 215, |
| | 216, 217, 218, 222, 223, 224, 225, 227, 228, 232, 236, 237, |
| | 238, 239, 243, 244, 245, 246, 247, 248, 251, 253, 254, 255, |
| | 256, 257, 258, 259, 260, 261, 262, 265, 275, 277, 279, 282, |
| | 283, 285, 287, 288, 289, 291, 293, 294, 297, 298, 299, 300, |
| | 301, 302, 305, 308, 314, 315, 316, 317, 319, 322, 323, 326, |
| | 328, 331, 332, 16 A and B, 17 A and B, 18 A and B, 22 A |
| | and B, 23 A and B, 43 B, 68 A and B, 69 A and B |
| +++ | 219, 4, 31, 40, 46, 51, 55, 56, 57, 66, 67, 75, 76, 80, 94, 107, |
| | 108, 111, 112, 116, 117, 118, 119, 120, 121, 122, 123, 126, |
| | 127, 132, 145, 163, 164, 166, 180, 187, 194, 199, 210, 212, |
| | 220, 221, 226, 229, 233, 235, 240, 242, 250, 286, 290, 292, |
| | 295, 296, 303, 304, 310, 313, 320, 321, 324, 15 A and B, 16 |
| | A and B, 17 A and B, 19 A and B, 20 A and B, 32 A and B, |
| | 42 B, 47 A and B, 61 A and B, 62 A and B, 70 A and B, 71 |
| | A and B, 72 A and B, 72 A and B, 79 A and B, 82 A and B |
| ++++ | 318, 26, 28, 29, 63, 81, 98, 234, 327, 18 A and B, 24 A and |
| | B, 47 A and B, 62 A and B, 68 A and B, 69 A and B, 82 A |
| | and B |
| +++++ | 25, 27, 30, 60, 99, 15 A and B, 19 A and B, 20 A and B, 24 |
| | aA nd B, 32 A and B, 61 A and B, 64 A and B, 64 A and B, |
| | 65 A and B, 65 A and B, 70 A and B, 71 A and B, 79 A and |
| | B |

TABLE 16
NRAS WT FRET data

| IC50* | Examples of A compounds |
|---|---|
| + | 258, 264, 315, 325 |
| ++ | 37, 91, 92, 117, 141, 167, 170, 186, 204, 205, 207, 208, 209, |
| | 213, 214, 217, 222, 223, 224, 225, 230, 231, 237, 238, 239, |
| | 241, 245, 246, 248, 249, 252, 253, 255, 256, 257, 259, 260, |
| | 261, 262, 263, 265, 267, 268, 269, 272, 273, 274, 276, 278, |
| | 279, 280, 281, 282, 283, 284, 285, 287, 288, 289, 291, 297, |
| | 298, 301, 302, 305, 306, 307, 308, 309, 311, 314, 316, 317, |
| | 322, 323, 326, 328, 329, 330, 331, 332 |
| +++ | 36, 108, 119, 144, 163, 164, 165, 166, 202, 203, 206, 211, |
| | 212, 215, 216, 218, 219, 220, 221, 226, 227, 228, 229, 232, |
| | 236, 242, 243, 244, 247, 250, 251, 254, 266, 270, 271, 275, |
| | 277, 286, 290, 292, 293, 294, 295, 296, 299, 300, 303, 304, |
| | 310, 312, 321, 324 |
| ++++ | 210, 233, 234, 235, 240, 313, 318, 319, 320 |
| +++++ | 327 |

TABLE 17
NRAS Q61K FRET data

| IC50* | Examples of A compounds |
|---|---|
| + | None |
| ++ | 167, 170, 216, 217, 218, 219, 220, 222, 223, 224, 225, 227, 228, |
| | 229, 231, 246, 249, 258, 260, 264, 281, 289, 291, 293, 294, 297, |
| | 298, 299, 300, 301, 302, 315, 325, 326, 329 |
| +++ | 36, 37, 91, 92, 108, 117, 119, 141, 163, 186, 202, 203, 204, 205, |
| | 207, 208, 209, 212, 213, 214, 221, 226, 230, 232, 235, 237, 238, |
| | 239, 241, 245, 248, 252, 253, 254, 255, 256, 257, 259, 261, 262, |
| | 263, 265, 266, 267, 268, 269, 271, 272, 273, 274, 275, 276, 277, |
| | 278, 279, 280, 282, 283, 284, 285, 286, 287, 288, 290, 292, 295, |
| | 296, 303, 304, 305, 306, 307, 308, 309, 310, 311, 314, 316, 317, |
| | 319, 322, 323, 324, 328, 330, 331, 332 |
| ++++ | 144, 164, 165, 166, 206, 210, 211, 215, 233, 234, 236, 242, 243, |
| | 244, 247, 250, 251, 270, 312, 313, 318, 320, 321 |
| +++++ | 240, 327 |

TABLE 18
NRAS Q61R FRET data

| IC50* | Examples of A compounds |
|---|---|
| + | None |
| ++ | 170, 209, 222, 223, 224, 230, 249, 258, 264, 289, 297, 298, 301, |
| | 302, 325, 326, 329 |
| +++ | 36, 37, 91, 92, 108, 117, 141, 163, 167, 186, 202, 203, 204, 205, |
| | 207, 208, 212, 213, 214, 216, 217, 218, 219, 220, 221, 225, 226, |
| | 227, 228, 229, 231, 232, 237, 238, 239, 241, 244, 245, 246, 248, |
| | 252, 253, 254, 255, 256, 257, 259, 260, 261, 262, 263, 265, 266, |
| | 267, 268, 269, 271, 272, 273, 274, 275, 276, 278, 279, 280, 281, |
| | 282, 283, 284, 285, 286, 287, 288, 290, 291, 292, 293, 294, 296, |
| | 299, 300, 303, 304, 305, 306, 307, 308, 309, 310, 311, 314, 315, |
| | 316, 317, 322, 323, 324, 328, 330, 331, 332 |
| ++++ | 119, 144, 164, 165, 166, 206, 210, 211, 215, 234, 235, 236, 240, |
| | 242, 243, 247, 250, 251, 270, 277, 295, 312, 313, 318, 319, 320, |
| | 321 |
| +++++ | 233, 327 |

Cross-Linking of Ras Proteins with Compounds of the Invention to Form Conjugates

Note—The following protocol describes a procedure for monitoring cross-linking of K-Ras G12C (GMP-PNP) to a compound of the invention. This protocol may also be executed substituting other Ras proteins or nucleotides, such as such as K-Ras G12D and K-Ras G13D.

The purpose of this biochemical assay was to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A, and 2 μM test compound, a 5 μM stock of GMP-PNP-loaded K-Ras (1-169) G12C was diluted 10-fold to yield a final concentration of 0.5 μM; with final sample volume being 100 μL.

The sample was incubated at 25° C. for a time period of up to 24 hours prior to quenching by the addition of 10 UL of 5% Formic Acid. Quenched samples were centrifuged at 15000 rpm for 15 minutes in a benchtop centrifuge before injecting a 10 μL aliquot onto a reverse phase C₄ column and eluting into the mass spectrometer with an increasing acetonitrile gradient in the mobile phase. Analysis of raw data was carried out using Waters MassLynx MS software, with % bound calculated from the deconvoluted protein peaks for labeled and unlabeled K-Ras.

### In Vitro Cell Proliferation Panels

Potency for inhibition of cell growth was assessed at CrownBio using standard methods. Briefly, cell lines were cultured in appropriate medium, and then plated in 3D methylcellulose. Inhibition of cell growth was determined by CellTiter-Glo® after 5 days of culture with increasing concentrations of compounds. Compound potency was reported as the 50% inhibition concentration (absolute IC50).

The assay took place over 7 days. On day 1, cells in 2D culture were harvested during logarithmic growth and suspended in culture medium at 1×105 cells/ml. Higher or lower cell densities were used for some cell lines based on prior optimization. 3.5 ml of cell suspension was mixed with 6.5% growth medium with 1% methylcellulose, resulting in a cell suspension in 0.65% methylcellulose. 90 μl of this suspension was distributed in the wells of 2 96-well plates. One plate was used for day 0 reading and 1 plate was used for the end-point experiment. Plates were incubated overnight at 37 C with 5% CO₂. On day 2, one plate (for to reading) was removed and 10 μl growth medium plus 100 μl CellTiter-Glo® Reagent was added to each well. After mixing and a 10 minute incubation, luminescence was recorded on an EnVision Multi-Label Reader (Perkin Elmer). Compounds in DMSO were diluted in growth medium such that the final, maximum concentration of compound was 10 μM, and serial 4-fold dilutions were performed to generate a 9-point concentration series. 10 μl of compound solution at 10 times final concentration was added to wells of the second plate. Plate was then incubated for 120 hours at 37C and 5% CO₂. On day 7 the plates were removed, 100 μl CellTiter-Glo® Reagent was added to each well, and after mixing and a 10 minute incubation, luminescence was recorded on an EnVision Multi-Label Reader (Perkin Elmer). Data was exported to GeneData Screener and modeled with a sigmoidal concentration response model in order to determine the IC50 for compound response. Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite having a KRAS G12C mutation, is insensitive to the KRAS G12C(OFF) inhibitor MRTX-849 (Hallin et al., Cancer Discovery 10:54-71 (2020)), and the cell line SW1573, which is insensitive to the KRAS G12C(OFF) inhibitor AMG510 (Canon et al., Nature 575:217-223 (2019)).

TABLE 19
IC50 values for various cancer cell lines with Compound B

| | | Cancer | |
|---|---|---|---|
| Cell Line | Histotype | Driver/Mutant | IC50* |
| A-375 | Skin | BRAF V600E | low sensitivity |
| KYSE-410 | HN/Esophagus | KRAS G12C | moderately |
| | | | sensitive |
| MIA PaCa-2 | Pancreas | KRAS G12C | very sensitive |
| NCI-H358 | Lung | KRAS G12C | very sensitive |
| SW1573 | Lung | KRAS G12C | low sensitivity |
| SW837 | Intestine/Large/ | KRAS G12C | very sensitive |
| | Colorectum | | |
| LS513 | Intestine/Large/ | KRAS G12D | moderately |
| | Colorectum | | sensitive |
| HuCCT1 | Liver/Bile duct | KRAS G12D | very sensitive |
| HCC1588 | Lung | KRAS G12D | low sensitivity |
| HPAC | Pancreas | KRAS G12D | very sensitive |
| AsPC-1 | Pancreas | KRAS G12D | moderately |
| | | | sensitive |
| AGS | Stomach | KRAS G12D | very sensitive |
| HEC-1-A | Uterus | KRAS G12D | very sensitive |
| SW403 | Intestine/Large/ | KRAS G12V | moderately |
| | Colorectum | | sensitive |
| NOZ | Liver/Bile duct | KRAS G12V | low sensitivity |
| NCI-H441 | Lung | KRAS G12V | moderately |
| | | | sensitive |
| NCI-H727 | Lung | KRAS G12V | moderately |
| | | | sensitive |
| OVCAR-5 | Ovary | KRAS G12V | moderately |
| | | | sensitive |
| Capan-2 | Pancreas | KRAS G12V | moderately |
| | | | sensitive |
| SW48 | Intestine/Large/ | not MAPK (PIK3CA | |
| | Colorectum | G914R, EGFR | |
| | | G719S) | |
| NCI-H2009 | Lung | other KRAS (G12A) | moderately |
| | | | sensitive |
| CAL-62 | HN/Thyroid | other KRAS (G12R) | low sensitivity |
| A549 | Lung | other KRAS (G12S) | low sensitivity |
| TOV-21G | Ovary | other KRAS (G13C) | low sensitivity |
| DV-90 | Lung | other KRAS (G13D) | low sensitivity |
| HCT116 | Intestine/Large/ | other KRAS (G13D) | moderately |
| | Colorectum | | sensitive |
| NCI-H747 | Intestine/Large/ | other KRAS (G13D) | moderately |
| | Colorectum | | sensitive |
| NCI-H460 | Lung | other KRAS (Q61H) | moderately |
| | | | sensitive |
| Calu-6 | Lung | other KRAS (Q61K) | moderately |
| | | | sensitive |
| SNU-668 | Stomach | other KRAS (Q61K) | moderately |
| | | | sensitive |
| OZ | Liver/Bile duct | other KRAS (Q61L) | moderately |
| | | | sensitive |
| SW948 | Intestine/Large/ | other KRAS (Q61L) | low sensitivity |
| | Colorectum | | |
| BxPC-3 | Pancreas | other MAPK (BRAF | low sensitivity |
| | | V487_P492delinsA) | |
| NCI-H1975 | Lung | other MAPK (EGFR | moderately |
| | | T790M, L858R) | sensitive |
| NCI-H3122 | Lung | other MAPK | moderately |
| | | (EML4-ALK | sensitive |
| | | (E13, A20)) | |
| YCC-1 | Stomach | other MAPK | |
| | | (KRAS Amp) | |
| MeWo | Skin | other MAPK | low sensitivity |
| | | (NF1 mut) | |
| NCI-H1838 | Lung | other MAPK | moderately |
| | | (NF1 mut) | sensitive |

*Key:
low sensitivity: IC50 ≥ 1 uM
moderately sensitive: 1 uM > IC50 ≥ 0.1 uM
very sensitive: IC50 < 0.1 uM
blank = not measured

In Vivo PD and Efficacy Data with Compound a, a Compound of the Present Invention FIG. 1A:

Methods: The human pancreatic adenocarcinoma HPAC KRAS G12D/wt xenograft model was used for a single-dose PD study. Compound A (AsPC-1 pERK K-Ras G12D EC50: 0.036 μM) was administered at 30 and 60 mg/kg by intraperitoneal injection (ip injection). The treatment groups with sample collections at various time points were summarized in Table 20 below. Tumor samples were collected to assess RAS/ERK signaling pathway modulation by measuring the mRNA level of human DUSP6 in qPCR assay.

TABLE 20
Summary of treatment groups, doses, and time points for single-dose
PD study using HPAC tumors.

| Compound/group | Dose/Regimen | | PD, n = 3/time point |
|---|---|---|---|
| Vehicle control | 10 | ml/kg ip | 1 h, 24 h |
| Compound A | 30 | mg/kg ip | 1 h, 4 h, 8 h, 24 h |
| Compound A | 60 | mg/kg ip | 1 h, 4 h, 6 h, 24 h |

Results: In FIG. 1A, Compound A at either 30 mg/kg or 60 mg/kg led to inhibition of DUSP6 mRNA levels in tumors at all time points tested, indicating strong MAPK pathway modulation. The inhibitory effects of Compound A on DUSP6 mRNA levels are durable even 24 hours after drug administration.

### FIG. 1B:

Methods: Effects of Compound A on tumor cell growth in vivo were evaluated in the human pancreatic adenocarcinoma HPAC KRAS G12D/wt xenograft model using female BALB/c nude mice (6-8 weeks old). Mice were implanted with HPAC tumor cells in PBS (3×106 cells/mouse) subcutaneously in the flank. Once tumors reached an average size of ˜150 mm3, mice were randomized to treatment groups to start the administration of test articles or vehicle. Compound A was administered by intraperitoneal injection once daily. Body weight and tumor volume (using calipers) was measured twice weekly until study endpoints.

Results: Single-agent Compound A administered at 10 mg/kg ip daily led to a TGI of 89.9% at Day 28, while both 30 mg/kg and 60 mg/kg Compound A dosed ip daily resulted in complete regression of all tumors in the group (complete regression defined as >85% tumor regression from baseline) at the end of treatment (Day 35 after treatment started) in HPAC CDX model with heterozygous KRAS G12D. The anti-tumor activity of all 3 tested doses of Compound A was statistically significant compared with control group (*** p<0.001, ordinary One-way ANOVA with multiple comparisons via a post-hoc Tukey's test).

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### APPENDIX D

### Ras Inhibitors

### Background

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, a drug directly targeting Ras is still not approved. Additional efforts are needed to uncover additional medicines for cancers driven by the various Ras mutations.

### Summary

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

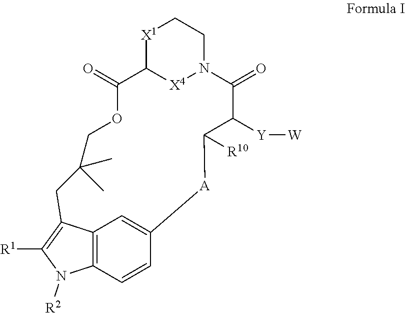

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

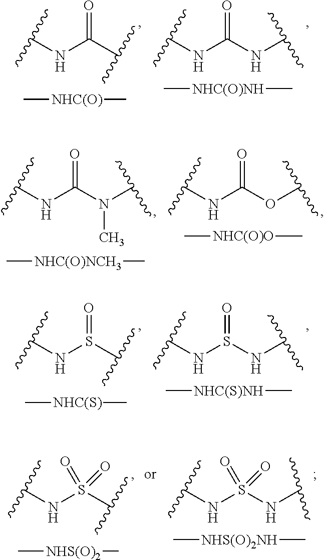

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 15-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen, hydroxy, optionally substituted C₁-C₃ alkyl, or optionally substituted C₁-C₆ heteroalkyl. In some embodiments, R¹⁰ is hydrogen.

Also provided are pharmaceutical compositions comprising a compound of Formula I, or a pharmaceutically acceptable salt thereof, and a pharmaceutical acceptable excipient.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, and compounds of Table 1 and Table 1-1, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium;
halogen; —(CH₂)₀₋₄R^{∘}; —(CH₂)₀₋₄OR^{∘}; —O (CH₂)₀₋₄R^{∘};
—O—(CH₂)₀₋₄C(O)OR^{∘}; —(CH₂)₀₋₄CH (OR^{∘})₂; —(CH₂)₀₋₄SR^{∘}; —(CH₂)₀₋₄Ph, which may be substituted with R^{∘}; —(CH₂)₀₋₄O (CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4 to 8-membered saturated or unsaturated heterocycloalkyl (e.g., pyridyl); 3 to 8-membered saturated or unsaturated cycloalkyl (e.g., cyclopropyl, cyclobutyl, or
cyclopentyl); —NO₂; —CN; —N₃; —(CH₂)₀₋₄N(R^{∘}))₂; —(CH₂)₀₋₄N(R^{∘}C(O)R^{∘};
—N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N (R^{∘})C(O)R^{∘}; —N(R^{∘})N(R^{∘})C(O)NR^{∘}₂; —N(R^{∘})N (R^{∘})C(O)OR^{∘}; —(CH₂)₀₋₄C(O)R^{∘}; —C(S)R^{∘}; —(CH₂)₀₋₄C(O)OR^{∘};)
—(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O) (CH₂)₀₋₄SR^{∘}; —SC(S)SR^{∘}; —(CH₂)₀₋₄SC(O)R^{∘}; —(CH₂)₀₋₄C(O)NR^{∘}₂;
—C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}); —N(R^{∘})S(O)₂NR^{∘)}₂; —N(R^{∘}S(O)₂R^{∘}; —N(OR^{∘})R^{∘}; —C(NOR^{∘}) NR^{∘}₂; —C(NH)NR^{∘}₂; —P(O)₂ R^{∘};
—P(O)R^{∘}₂; —P(O)(OR^{∘})₂; —OP(O)R^{∘}₂; —OP(O)(OR^{∘})₂; —OP(O)(OR^{∘}) R^{∘}, —SiR^{∘}₃; —(C₁-C₄ straight or branched) alkylene)O—N(R^{∘}₂; or —(C₁-C₄ straight or branched) alkylene) C(O)O—N(R^{∘}₂, wherein each R^{∘} may be substituted as defined below and is independently hydrogen, —C₁-C₆ aliphatic, —CH₂Ph, —O(CH₂)₀₋₁Ph, —CH₂-(5 to 6 membered heteroaryl ring), or a 3 to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3 to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•},
(haloR^{•}), —(CH₂)₀₋₂OH, —(CH₂)₀₋₂OR^{•}, —(CH₂)₀₋₂CH (OR^{•})₂; —O (haloR^{•}), —CN, —N₃, —(CH₂)₀₋₂C(O)R^{•}, —(CH₂)₀₋₂C(O)OH,
(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{∘}, —(C₁₋₄ straight or branched alkylene )C(O)OR′, or —SSR, wherein each R^{•} is unsubstituted or where preceded by “halo” is substituted only with one or more halogens, and is independently selected from C₁-C₄ aliphatic, —CH₂Ph, —O (CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*,
—O (C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include
halogen, —R^{•}, -(haloR^{•}), —OH, —OR^{•},
—O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently
C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)Rt, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rt) S(O)₂R^{†}; wherein each R^{†} is independently hydrogen, C₁-C₆ aliphatic which may be substituted as defined below, unsubstituted —OPh, or an unsubstituted 3 to 6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3 to 12-membered saturated, partially unsaturated, or aryl mono- or bicyclic ring having 0-4 heteroatoms independently selected from nitrogen, oxygen, or sulfur.

Suitable substituents on an aliphatic group of Rt are independently halogen, —R^{•}, -(haloR^{•}), —OH,
—OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

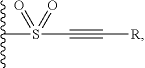

wherein R is any chemically feasible substituent described herein.

The term “amino,” as used herein, represents-N(Rt)₂, e.g., —NH₂ and —N(CH₃) 2.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term-N(C(O)—(C₀-C₅ alkylene-H)-includes-N(C(O)—(Co alkylene-H)—, which is also represented by-N (C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

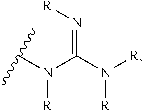

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more-OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

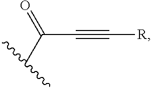

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### BRIEF DESCRIPTION OF THE DRAWINGS

FIG. 1A is a graph demonstrating the in vivo efficacy of Compound A in a human pancreatic adenocarcinoma HPAC KRAS^{G12D/wt} xenograft model using female BALB/c nude mice. The graph shows tumor volume (mm³) vs. days post-implant of the mouse xenograft model. Mice were randomized to treatment groups prior to administration of test articles or vehicle. Compound A was administered by oral gavage once every other day.

FIG. 1B is a graph demonstrating dose-dependent exposure of Compound A in blood and tumor samples from BALB/c nude mice (6-8 weeks old, human non-small cell lung cancer (NSCLC) NCI-H441 KRAS^{G12V/wt} xenograft model), monitored through 72 hours following dose. Pharmacokinetics were analyzed based on total concentration (nM) of Compound A in tumors or blood, following a single oral gavage dose of Compound A at 10, 25 or 50 mg/kg to 72 hours. At each time point, tumor or blood was sampled from n=3 animals.

FIG. 1C is a graph showing PK (10 mg/kg, po) and PD (% tumor DUSP relative to control, 10 and 25 mg/kg, po) in naïve animals treated with a single dose of Compound A.

FIG. 1D is a graph demonstrating the in vivo efficacy of Compound A in the NCI-H441 CDX model with heterozygous KRASG12V. NCI-H₄₄₁ cells were implanted in 50% Matrigel. Animals were randomized and treatment was initiated at average tumor volume of ˜155 mm³. Animals were dosed with Compound A 10 or 25 mg/kg po qd or Control for 32 days. All dose levels were tolerated. n=10/group. *** p<0.0001 by one-way ANOVA.

FIG. 1E shows end of study responses for Compound A in the NCI-H441 CDX model with heterozygous KRASG12V. NCI-H441 end of study tumors were graphed as % change in tumor volume compared to volume at treatment initiation. R (regressions)=number of regressions >10% from initial. CR (complete response)=number of regressions >80% from initial. Each animal represented as a separate bar.

FIG. 1F shows shows % body weight change in animals treated with Compound A (in the NCI-H441 CDX model with heterozygous KRAS^{G12V}). NCI-H441 cell-derived xenografts were measured twice weekly by caliper measurements. Body weight change graphed as percentage of animals starting body weight.

FIG. 2A is a graph demonstrating in vivo efficacy of Compound A in the human pancreatic Capan-2 CDX xenograft model with heterozygous KRAS^{G12V} using female BALB/c nude mice. The graph shows tumor volume (mm³) vs. days post-implant of the mouse xenograft model. Capan-2 cells were implanted in 50% Matrigel. Animals were randomized and treatment was initiated at average tumor volume of ˜166 mm³. Animals were dosed with Compound A 10 mg/kg po qd or 25 mg/kg po q2d or Control for 28 days. All dose levels were tolerated. n=8/group. ** p=0.01, *** p<0.0001 by one-way ANOVA.

FIG. 2B shows end of study responses for Compound A in the human pancreatic Capan-2 CDX xenograft model with heterozygous KRAS^{G12V}. Capan-2 end of study tumors were graphed as % change in tumor volume compared to volume at treatment initiation. R (regressions)=number of regressions >10% from initial. Each animal represented as a separate bar.

FIG. 2C shows % body weight change in animals treated with Compound A in the human pancreatic Capan-2 CDX xenograft model with heterozygous KRAS^{G12V}. Capan-2 cell-derived xenografts were measured twice weekly by caliper measurements. Body weight change graphed as percentage of animals starting body weight.

FIG. 2D is a graph demonstrating in vivo efficacy of Compound A in the human colorectal SW403 KRAS^{G12V/wt} xenograft model using female BALB/c nude mice. The graph shows tumor volume (mm³) vs. days post-implant of the mouse xenograft model. SW403 cells were implanted in 50% Matrigel. Animals were randomized and treatment was initiated at average tumor volume of ˜171 mm³. Animals were dosed with Compound A 25 mg/kg po qd or 50 mg/kg po q2d or Control for 28 days. All dose levels were tolerated. n=8/group. *** p<0.0001 by one-way ANOVA.

FIG. 2E shows end of study responses for Compound A in the human colorectal SW403 KRAS^{G12V/wt} xenograft model. SW403 end of study tumors were graphed as % change in tumor volume compared to volume at treatment initiation. R (regressions)=number of regressions >10% from initial. CR (complete response)=number of regressions >80% from initial. Each animal represented as a separate bar.

FIG. 2F shows % body weight change in animals treated with Compound A in the human colorectal SW403 KRAS^{G12V/wt} xenograft model. SW403 cell-derived xenografts were measured twice weekly by caliper measurements. Body weight change graphed as percentage of animals starting body weight.

FIG. 3 demonstrates in vitro efficacy of Compound A in multiple RAS-driven cancer cell lines. Each graph shows cell proliferation (% relative to control) vs. log M [Compound A]. Potency of in vitro cell proliferation inhibition of Capan-1 (KRAS^{G12V}), AsPC-1 (KRAS^{G12D}), HCT116 (KRAS^{G13D}), SK-MEL-30 (NRAS^{Q61K}), NCI-H1975 (EGFR^{T790M/L858R}), and A375 (BRAF^{V600E}) cells exposed to Compound A for 120 hours. Data represent the mean of multiple experiments.

FIG. 4A demonstrates in vivo efficacy of Compound A (25 mg/kg po qd) in the human pancreatic adenocarcinoma HPAC KRAS^{G12D/wt} xenograft model using female BALB/c nude mice. The graph shows tumor volume (mm³) vs. days post-implant of the mouse xenograft model. HPAC cells were implanted in 50% Matrigel. Animals were randomized and treatment was initiated at average tumor volume of ˜142 mm³. Animals were dosed with Compound A 25 mg/kg po qd or Control for 28 days. Dose level was tolerated. n=9-10/group. *** p<0.0001 by one-way ANOVA.

FIG. 4B shows end of study responses for Compound A in the human pancreatic adenocarcinoma HPAC KRAS^{G12D/wt} xenograft model. HPAC end of study tumors were graphed as % change in tumor volume compared to volume at treatment initiation. CR (complete response)=number of regressions >80% from initial. Each animal represented as a separate bar.

FIG. 4C shows % body weight change in animals treated with Compound A in the human pancreatic adenocarcinoma HPAC KRAS^{G12D/wt} xenograft model. HPAC cell-derived xenografts were measured twice weekly by caliper measurements. Body weight change graphed as percentage of animals starting body weight.

FIG. 4D demonstrates in vivo efficacy of Compound A in the human colorectal GP2d KRAS^{G12D/wt} xenograft model using female BALB/c nude mice. The graph shows tumor volume (mm³) vs. days post-implant of the mouse xenograft model. GP2d cells were implanted in 50% Matrigel. Animals were randomized and treatment was initiated at average tumor volume of ˜154 mm³. Animals were dosed with Compound A 25 mg/kg po qd or Control for 28 days. Dose level was tolerated. n=10/group. *** p<0.0001 by one-way ANOVA.

FIG. 4E shows end of study responses for Compound A in the human colorectal GP2d KRAS^{G12D/wt} xenograft model. GP2d end of study tumors were graphed as % change in tumor volume compared to volume at treatment initiation. Each animal represented as a separate bar.

FIG. 4F shows % body weight change in animals treated with Compound A in the human colorectal GP2d KRAS^{G12D/wt} xenograft model. GP2d cell-derived xenografts were measured twice weekly by caliper measurements. Body weight change graphed as percentage of animals starting body weight.

FIG. 5A demonstrates in vitro efficacy of Compound A in down-regulating immune checkpoint proteins in NCI-H358 KRAS^{G12C} Cells in Vitro. FIG. 5A shows cell surface expression of PD-L1, PVR and CD73 on H358 cells following 48-hour treatment with Compound A in the presence of Interferon gamma (IFNγ), as measured by flow cytometry. Each graph shows mean fluorescence intensity ((MFI), for each respective immune checkpoint protein) vs. log M [Compound A].

FIG. 5B demonstrates in vitro efficacy of Compound A in down-regulating immune checkpoint proteins in SW900 KRAS^{G12C} Cells in Vitro. FIG. 5B shows cell surface expression of PD-L1, PVR and CD73 on SW900 cells following 48-hour treatment with Compound A in the presence of Interferon gamma (IFNγ), as measured by flow cytometry. Each graph shows mean fluorescence intensity ((MFI), for each respective immune checkpoint protein) vs. log M [Compound A].

FIG. 5C demonstrates in vitro efficacy of Compound A in down-regulating immune checkpoint proteins in Capan-2 KRAS^{G12C} Cells in Vitro. FIG. 5C shows cell surface expression of PD-L1, PVR and CD73 on Capan-2 cells following 48-hour treatment with Compound A in the presence of Interferon gamma (IFNγ), as measured by flow cytometry. Each graph shows mean fluorescence intensity ((MFI), for each respective immune checkpoint protein) vs. log M [Compound A].

FIGS. 6A-6B demonstrate that Compound A, a KRAS^{MULTI}(ON) inhibitor disclosed herein, is active against RAS oncogene switching mutations observed in KRAS^{G12C}(OFF) resistance. FIG. 6A is a heatmap representing cellular RAS/RAF disruption assay results regarding various KRAS mutations in the presence of different RAS inhibitors. FIG. 6B shows the IC50 value associated with each colored bar of the heatmap.

### Detailed Description

### Compounds

Provided herein are Ras inhibitors. The approach described herein entails formation of a high affinity three-component complex between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex between the Ras protein and the widely expressed cytosolic chaperone, cyclophilin A (CYPA). Without being bound by theory, the inventors believe that one way the inhibitory effect on Ras is effected by compounds of the invention and the complexes they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) may contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., a wild-type Ras or Ras^{amp}, or K-Ras, N-Ras, H-Ras, and mutants thereof at positions 12, 13 and 61, such as G12C, G12D, G12V, G12S, G13C, G13D, and Q61L, and others described herein, as well as combinations of Ras proteins).

Accordingly, provided herein are compounds, or pharmaceutically acceptable salts thereof, having the structure of Formula I:

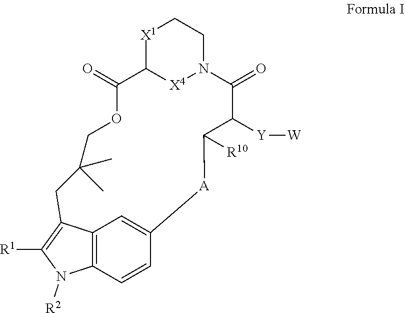

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

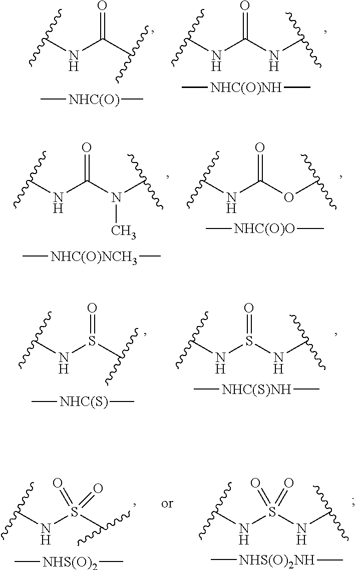

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the compound, or pharmaceutically acceptable salt thereof, has the structure of Formula Ia:

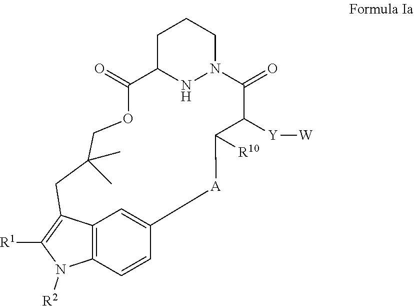

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
Y is

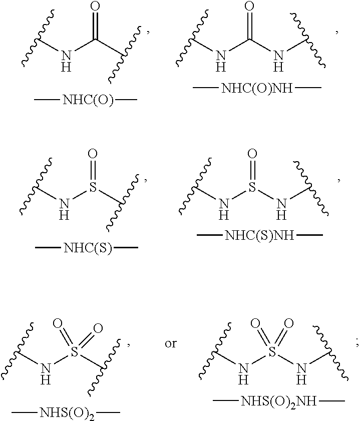

W is hydrogen, C₁-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl. In some embodiments, R¹⁰ is hydrogen.

In some embodiments, R¹ is optionally substituted 6 to 10-membered aryl or optionally substituted 5 to 10-membered heteroaryl. In some embodiments, R¹ is optionally substituted phenyl or optionally substituted pyridine.

In some embodiments, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments, Y is —NHC(O)— or —NHC(O)NH—.

In some embodiments, the compound, or a pharmaceutically acceptable salt thereof, has the structure of Formula II:

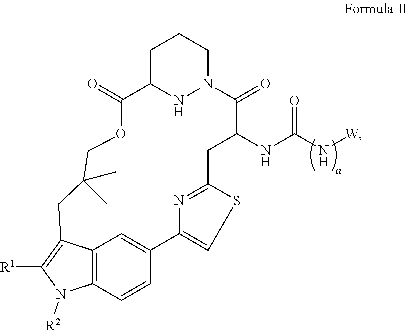

wherein a is 0 or 1.

In some embodiments, the compound, or a pharmaceutically acceptable salt thereof, has the structure of Formula II-1:

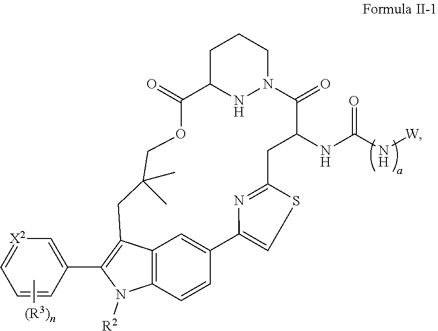

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula II-2:

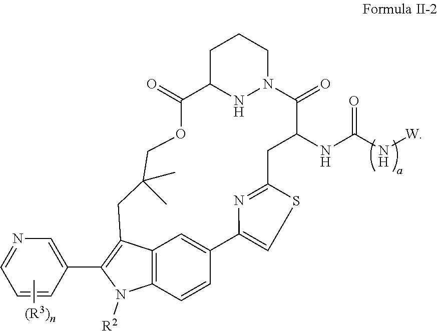

In some embodiments, the compound, or a pharmaceutically acceptable salt thereof, has the structure of Formula II-3:

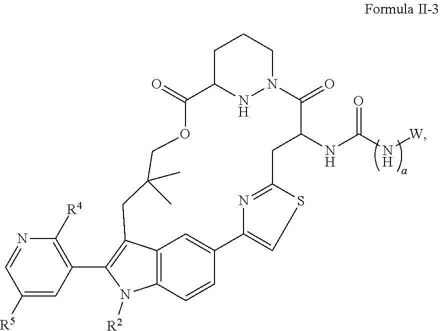

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula II-4:

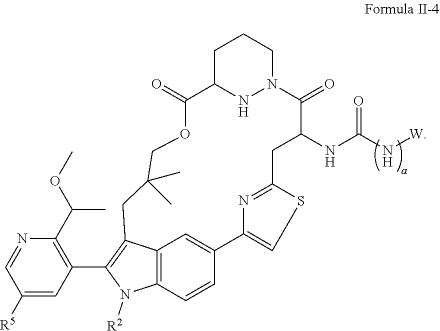

In some embodiments, the compound, or a pharmaceutically acceptable salt thereof, has the structure of Formula II-5:

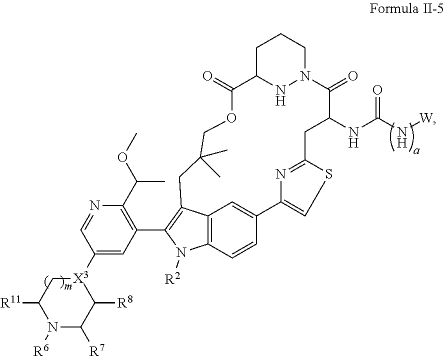

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the compound, or a pharmaceutically acceptable salt thereof, has the structure of Formula II-6:

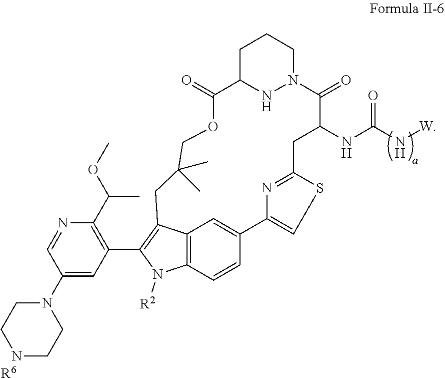

5 In some embodiments, the compound, or a pharmaceutically acceptable salt thereof, has the structure of Formula II-7:

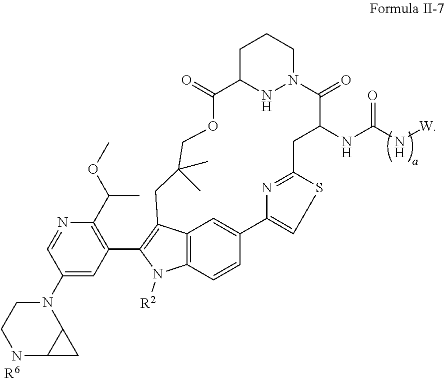

In some embodiments (e.g., of any one of Formulae II-6 or II-7), R⁶ is methyl.

In some embodiments, the compound, or a pharmaceutically acceptable salt thereof, has the structure of Formula II-8 or Formula II-9:

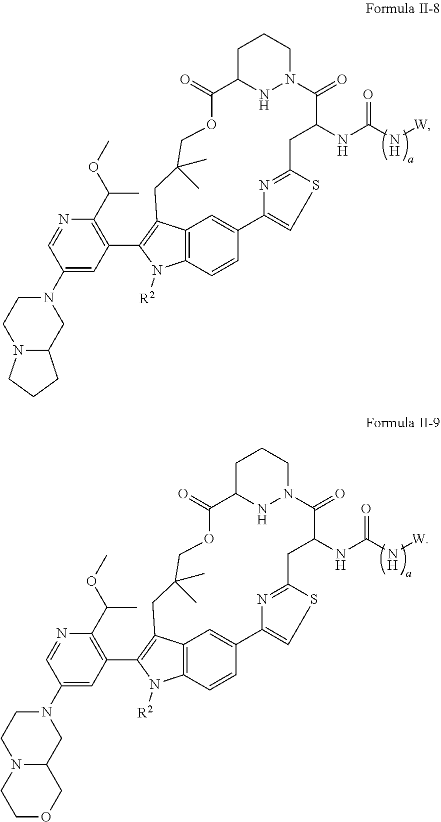

In some embodiments, the compound, or a pharmaceutically acceptable salt thereof, has the structure of Formula III:

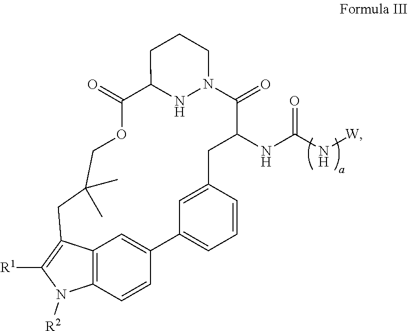

wherein a is 0 or 1.

In some embodiments, the compound, or a pharmaceutically acceptable salt thereof, has the structure of Formula III-1:

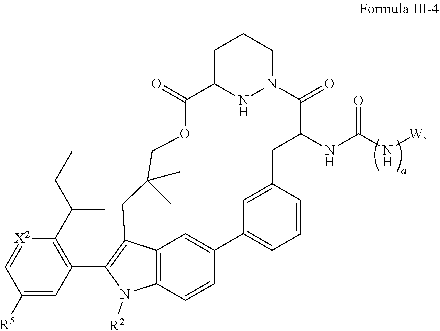

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula III-2:

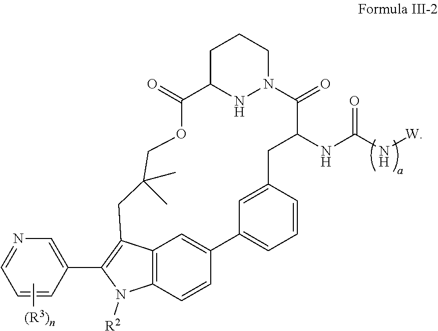

In some embodiments, the compound, or a pharmaceutically acceptable salt thereof, has the structure of Formula III-3:

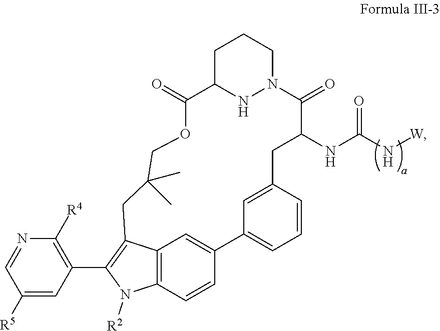

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula III-4:

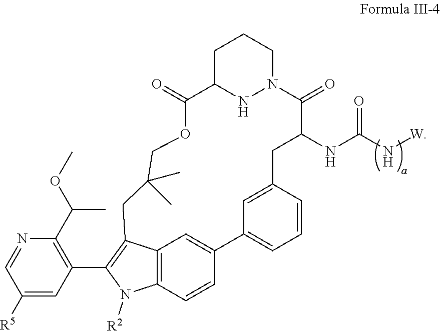

In some embodiments, the compound, or a pharmaceutically acceptable salt thereof, has the structure of Formula III-5:

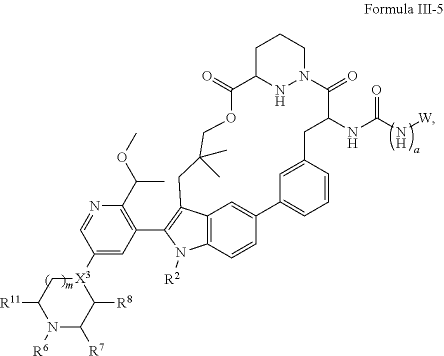

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula III-6:

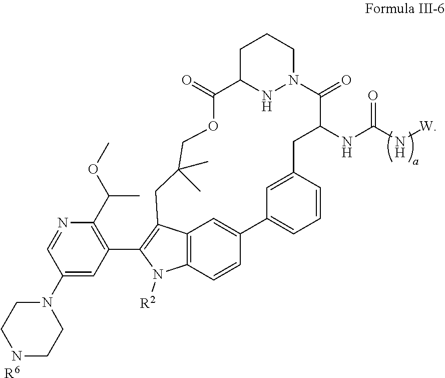

In some embodiments, the compound, or a pharmaceutically acceptable salt thereof, has the structure of Formula III-7:

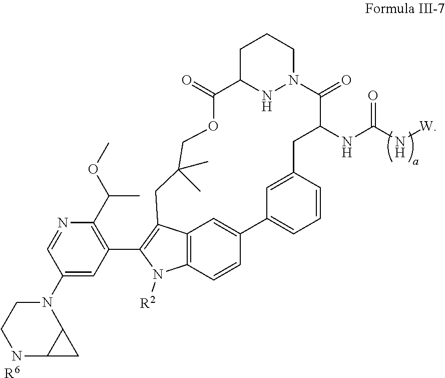

In some embodiments (e.g., of any one of Formulae III-6 or III-7), R⁶ is methyl.

In some embodiments, the compound, or a pharmaceutically acceptable salt thereof, has the structure of Formula III-8 or Formula III-9:

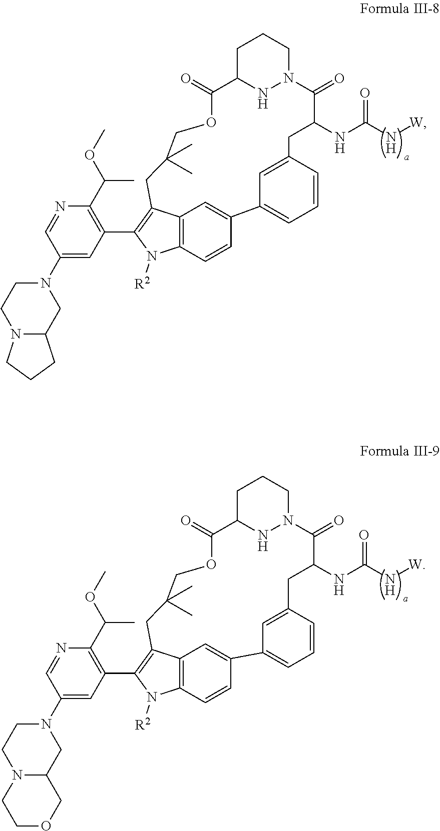

In some embodiments, the compound, or a pharmaceutically acceptable salt thereof, has the structure of Formula IV:

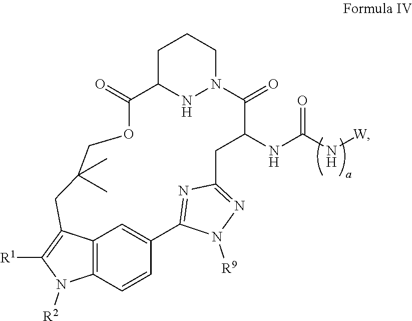

wherein R⁹ is H or C₁-C₆ alkyl; and
a is 0 or 1.

In some embodiments, the compound, or a pharmaceutically acceptable salt thereof, has the structure of Formula IV-1:

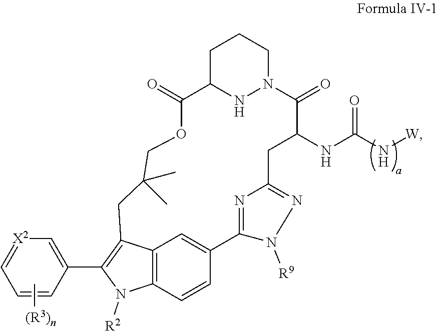

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula IV-2:

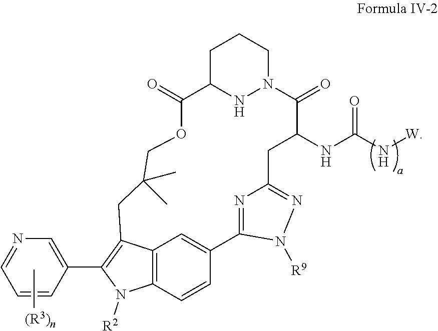

In some embodiments, the compound, or a pharmaceutically acceptable salt thereof, has the structure of Formula IV-3:

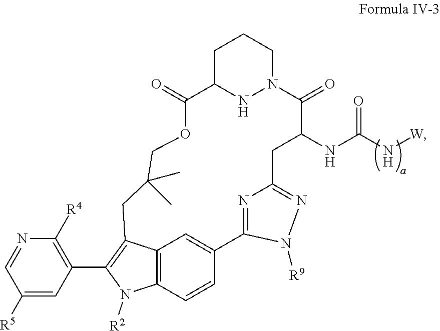

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula IV-4:

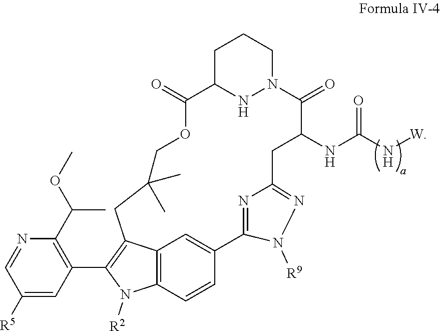

In some embodiments, the compound, or a pharmaceutically acceptable salt thereof, has the structure of Formula IV-5:

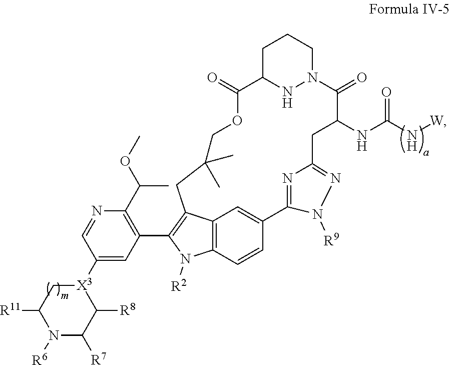

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula IV-6:

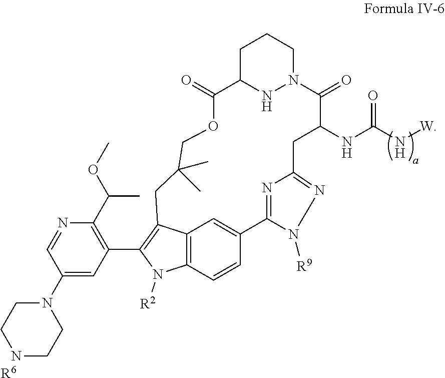

In some embodiments, the compound, or a pharmaceutically acceptable salt thereof, has the structure of Formula IV-7:

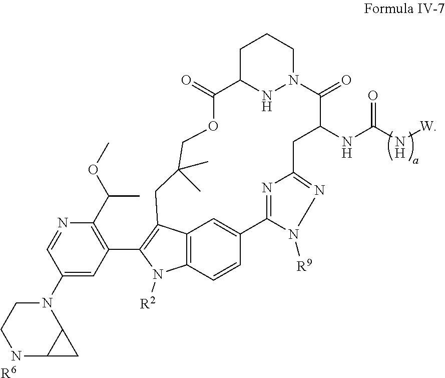

In some embodiments (e.g., of any one of Formulae IV-6 or IV-7), R⁶ is methyl.

In some embodiments, the compound, or a pharmaceutically acceptable salt thereof, has the structure of Formula IV-8 or Formula IV-9:

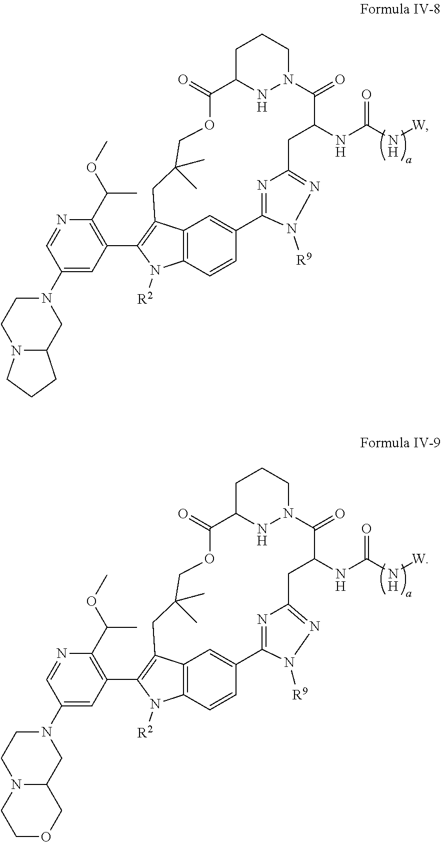

In some embodiments (e.g., of any one of Formulae IV, IV-1, IV-2, IV-3, IV-4, IV-5, IV-6, IV-7, IV-8, or IV-9), R⁹ is methyl.

In some embodiments, Y is —NHS(O)₂— or —NHS(O)₂NH—.

In some embodiments, the compound, or a pharmaceutically acceptable salt thereof, has the structure of Formula V:

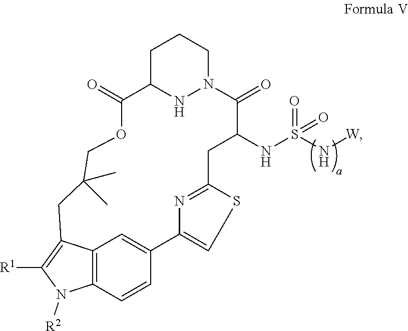

wherein a is 0 or 1.

In some embodiments, the compound, or a pharmaceutically acceptable salt thereof, has the structure of Formula V-1:

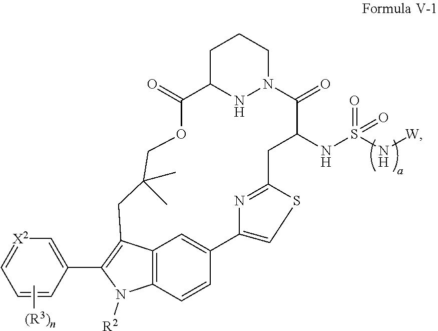

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula V-2:

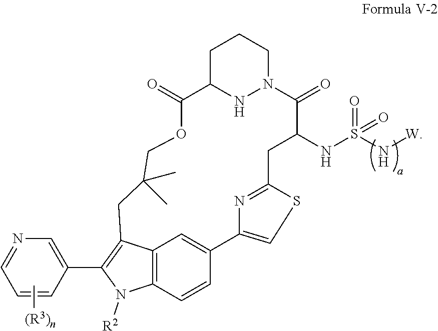

In some embodiments, the compound, or a pharmaceutically acceptable salt thereof, has the structure of Formula V-3:

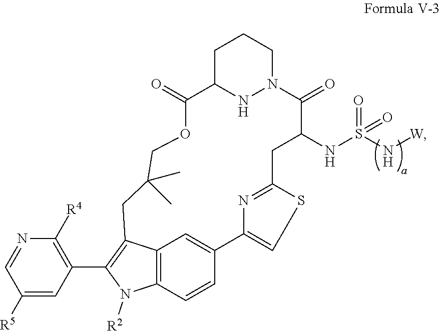

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula V-4:

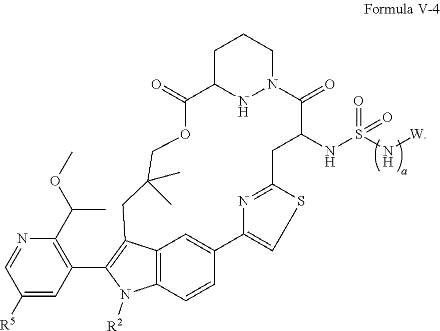

In some embodiments, the compound, or a pharmaceutically acceptable salt thereof, has the structure of Formula V-5:

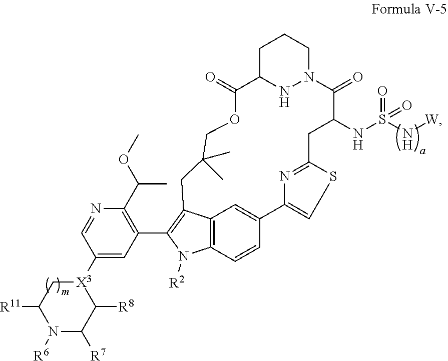

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula VI:

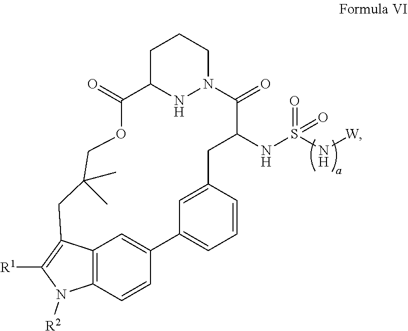

wherein a is 0 or 1.

In some embodiments, the compound, or a pharmaceutically acceptable salt thereof, has the structure of Formula VI-1:

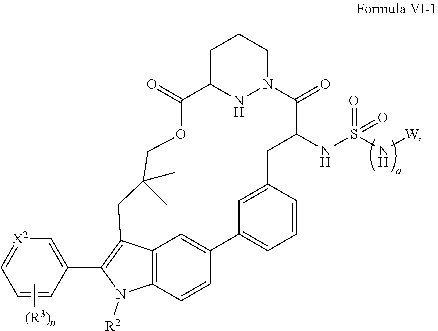

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula VI-2:

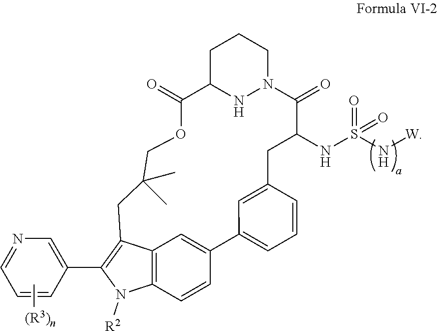

In some embodiments, the compound, or a pharmaceutically acceptable salt thereof, has the structure of Formula VI-3:

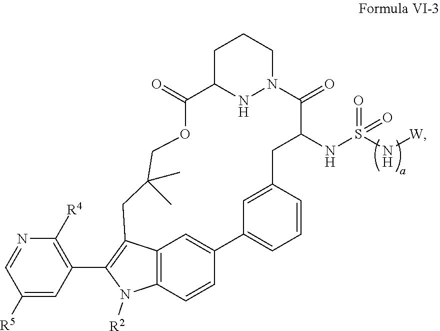

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula VI-4:

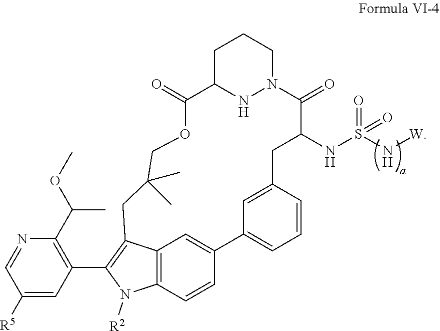

In some embodiments, the compound, or a pharmaceutically acceptable salt thereof, has the structure of Formula VI-5:

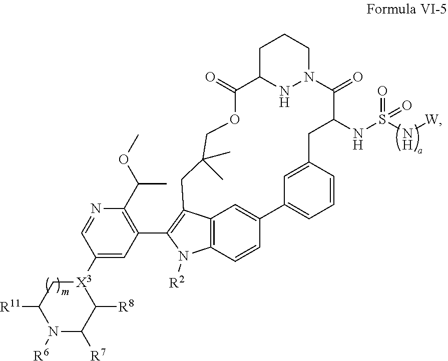

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula VII:

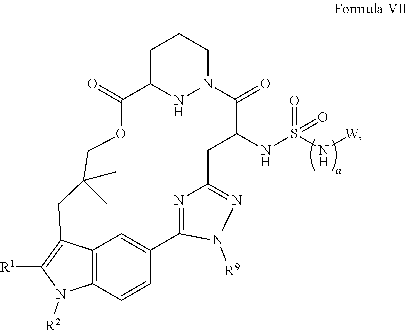

wherein R⁹ is H or C₁-C₆ alkyl; and
a is 0 or 1.

In some embodiments, the compound, or a pharmaceutically acceptable salt thereof, has the structure of Formula VII-1:

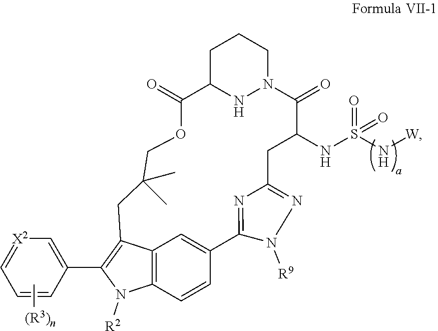

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula VII-2:

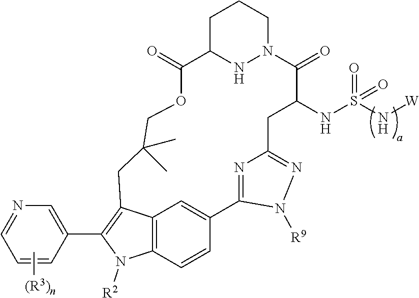

In some embodiments, the compound, or a pharmaceutically acceptable salt thereof, has the structure of Formula VII-3:

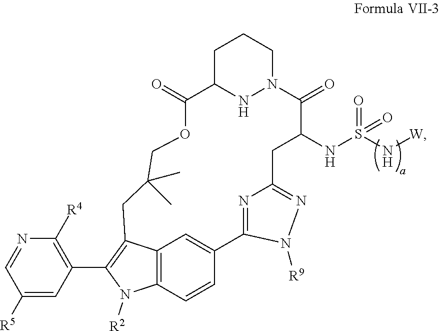

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula VII-4:

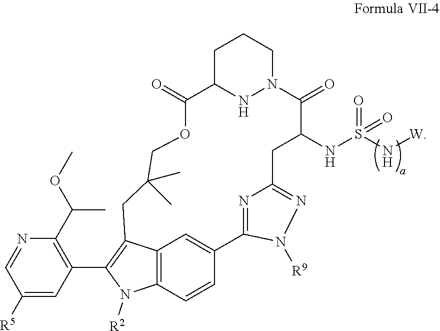

In some embodiments, the compound, or a pharmaceutically acceptable salt thereof, has the structure of Formula VII-5:

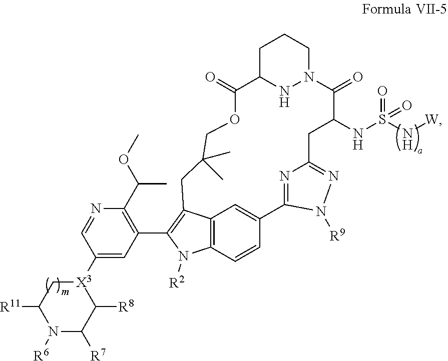

wherein X³ is N or CH;
m is 1 or 2;
R⁶, R⁷, R⁸, and R¹¹ are each independently selected from hydrogren, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae VII, VII-1, VII-2, VII-3, VII-4, or VII-5), R⁹ is methyl.

In some embodiments, Y is —NHS(O)— or —NHS(O)NH—.

In some embodiments, the compound, or a pharmaceutically acceptable salt thereof, has the structure of Formula VIII:

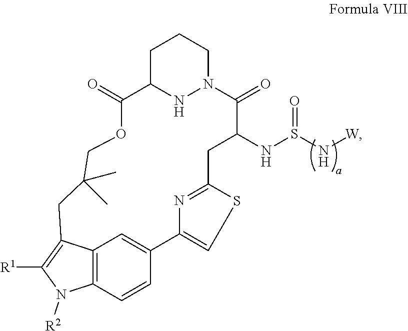

wherein a is 0 or 1.

In some embodiments, the compound, or a pharmaceutically acceptable salt thereof, has the structure of Formula VIII-1:

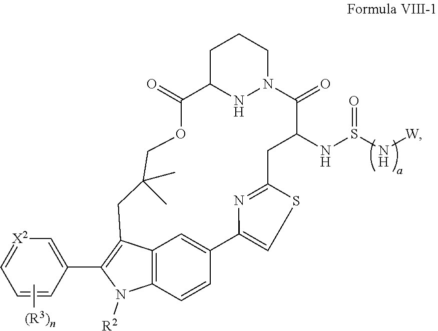

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula VIII-2:

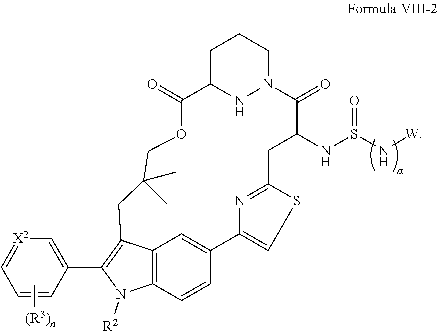

In some embodiments, the compound, or a pharmaceutically acceptable salt thereof, has the structure of Formula VIII-3:

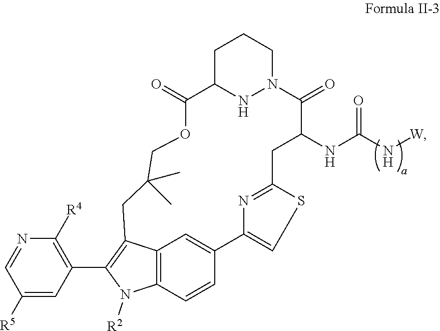

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula VIII-4:

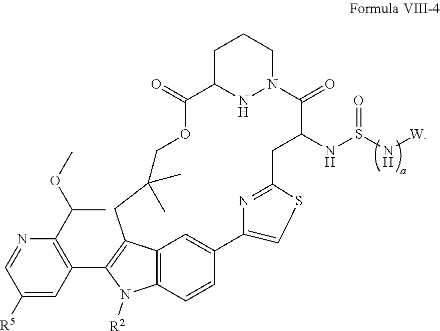

In some embodiments, the compound, or a pharmaceutically acceptable salt thereof, has the structure of Formula VIII-5:

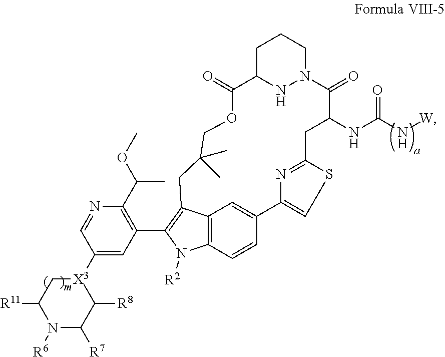

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula IX:

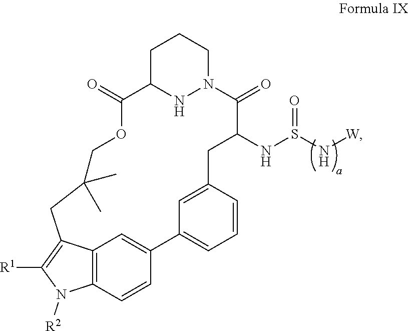

wherein a is 0 or 1.

In some embodiments, the compound, or a pharmaceutically acceptable salt thereof, has the structure of Formula IX-1:

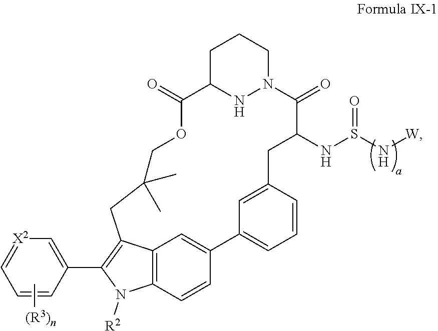

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula IX-2:

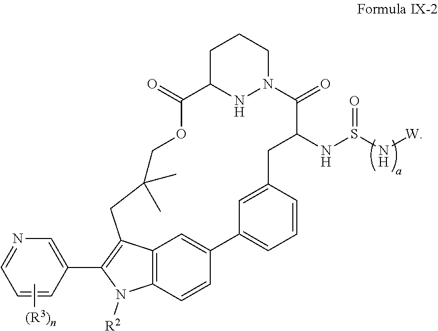

In some embodiments, the compound, or a pharmaceutically acceptable salt thereof, has the structure of Formula IX-3:

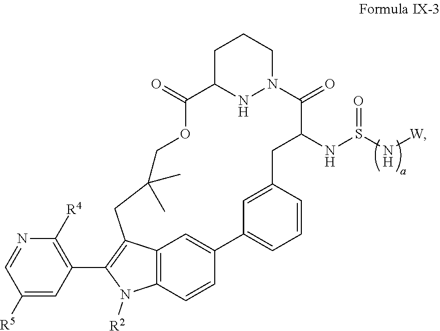

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula IX-4:

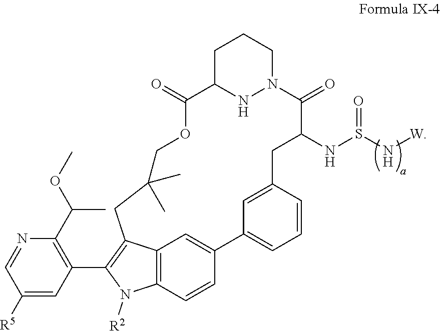

In some embodiments, the compound, or a pharmaceutically acceptable salt thereof, has the structure of Formula IX-5:

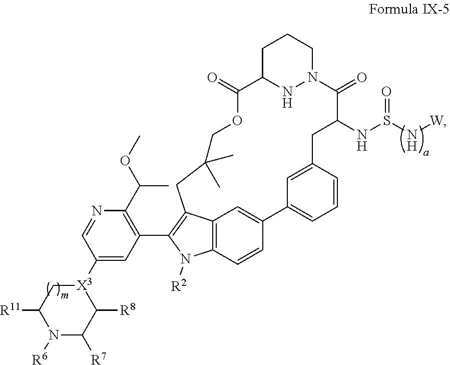

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound, or a pharmaceutically acceptable salt thereof, has the structure of Formula X:

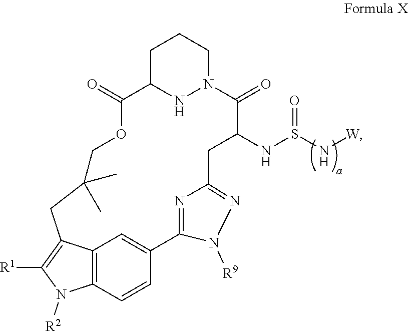

wherein R⁹ is H or C₁-C₆ alkyl; and
a is 0 or 1.

In some embodiments, the compound, or a pharmaceutically acceptable salt thereof, has the structure of Formula X-1:

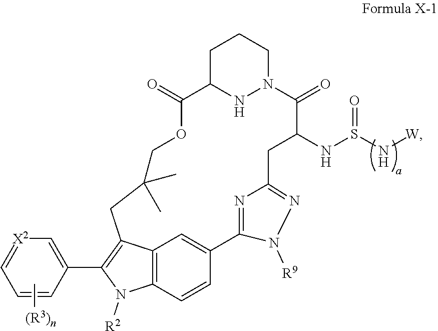

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the compound, or a pharmaceutically acceptable salt thereof, has the structure of Formula X-2:

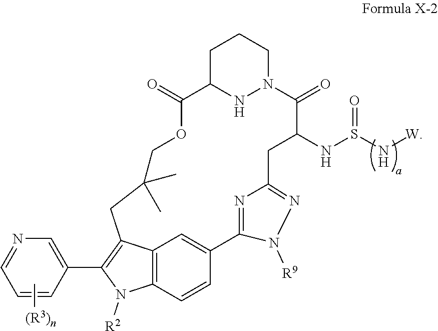

In some embodiments, the compound, or a pharmaceutically acceptable salt thereof, has the structure of Formula X-3:

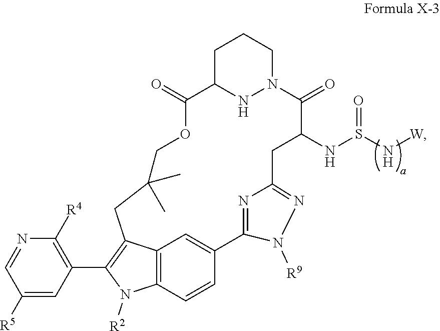

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the compound, or a pharmaceutically acceptable salt thereof, has the structure of Formula X-4:

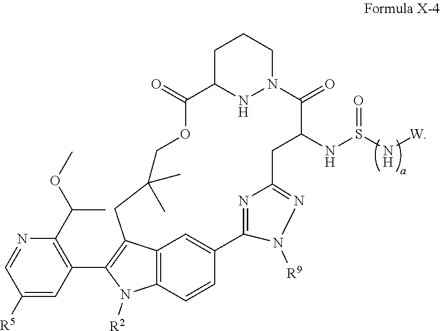

In some embodiments, the compound, or a pharmaceutically acceptable salt thereof, has the structure of Formula X-5:

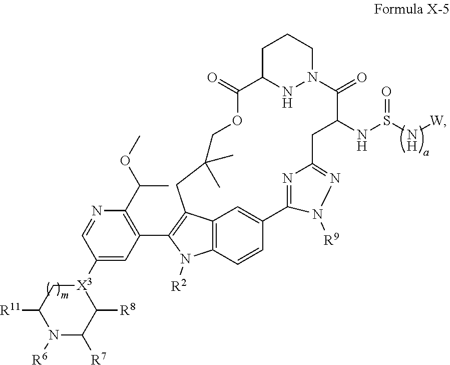

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae X, X-1, X-2, X-3, X-4, or X-5), R⁹ is methyl.

In some embodiments of any aspect described herein, a is 0. In some embodiments of any of the above, a is 0.

In some embodiments of any aspect described herein, R² is optionally substituted C₁-C₆ alkyl. In some embodiments, R² is selected from —CH₂CH₃ or —CH₂CF₃.

In some embodiments of any aspect described herein, W is C₁-C₄ alkyl. In some embodiments, W is:

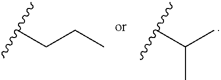

In some embodiments of any aspect described herein, W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.

In some embodiments of any aspect described herein, W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.

In some embodiments of any aspect described herein, W is optionally substituted 3 to 10-membered heterocycloalkyl. In some embodiments, W is selected from the following, or a stereoisomer thereof:

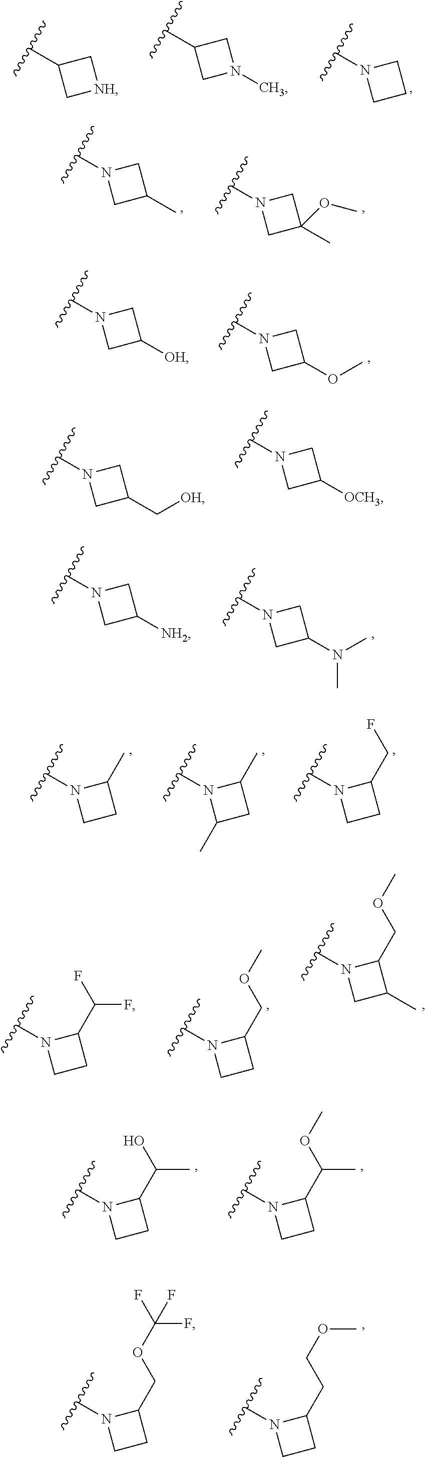

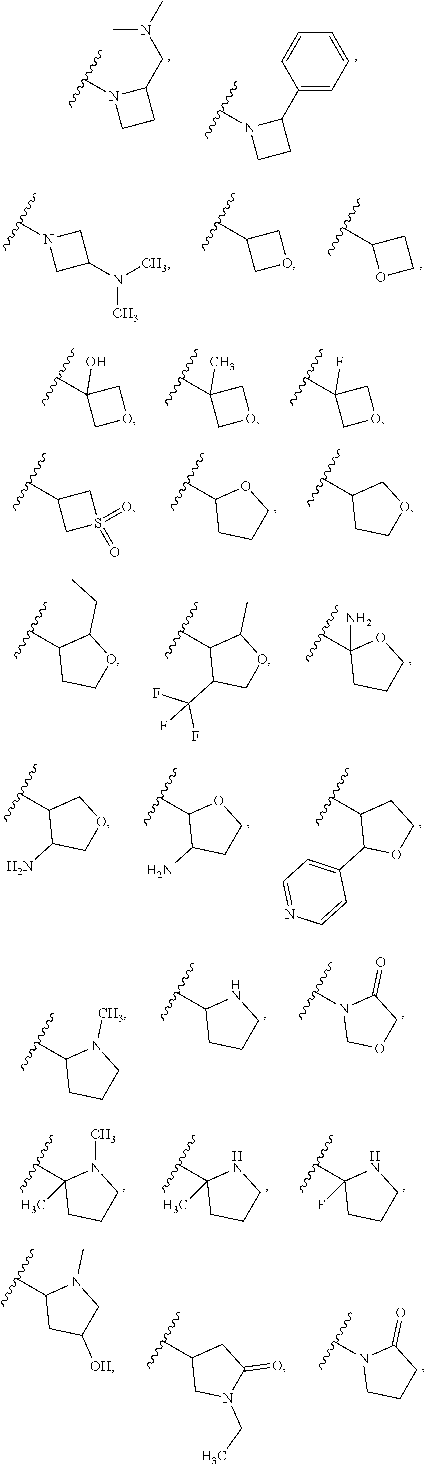

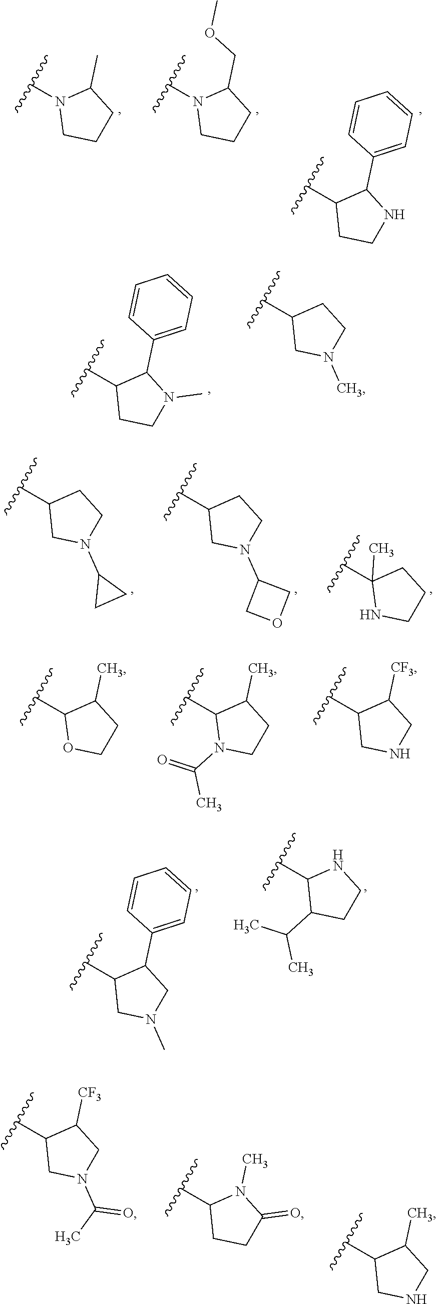

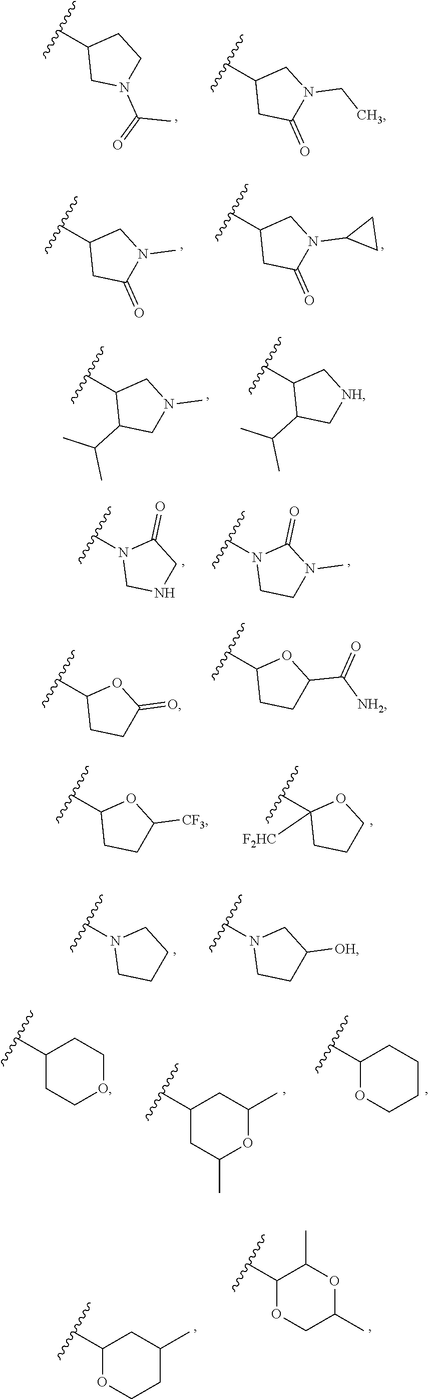

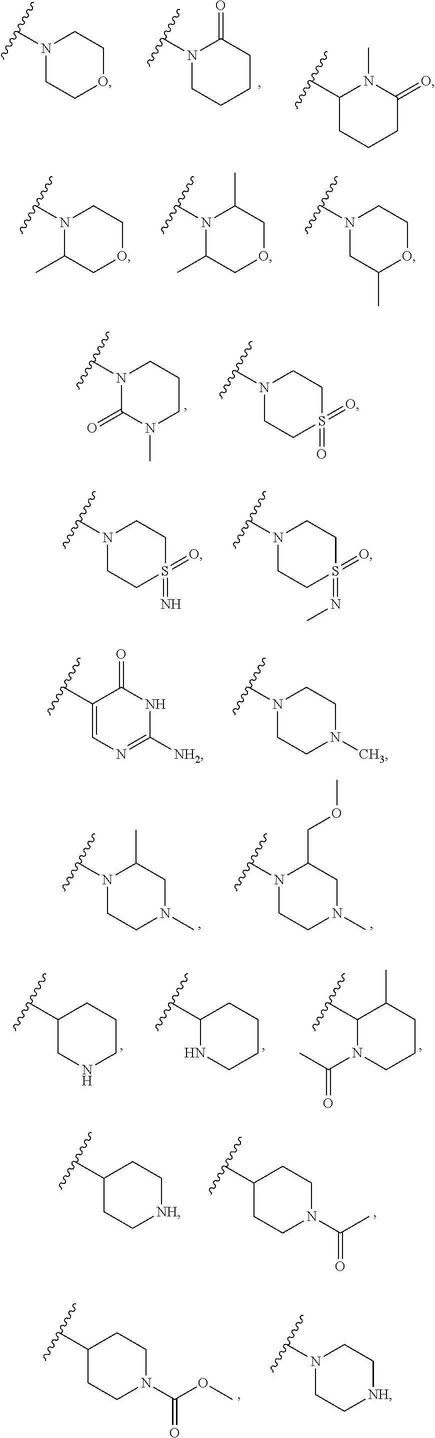

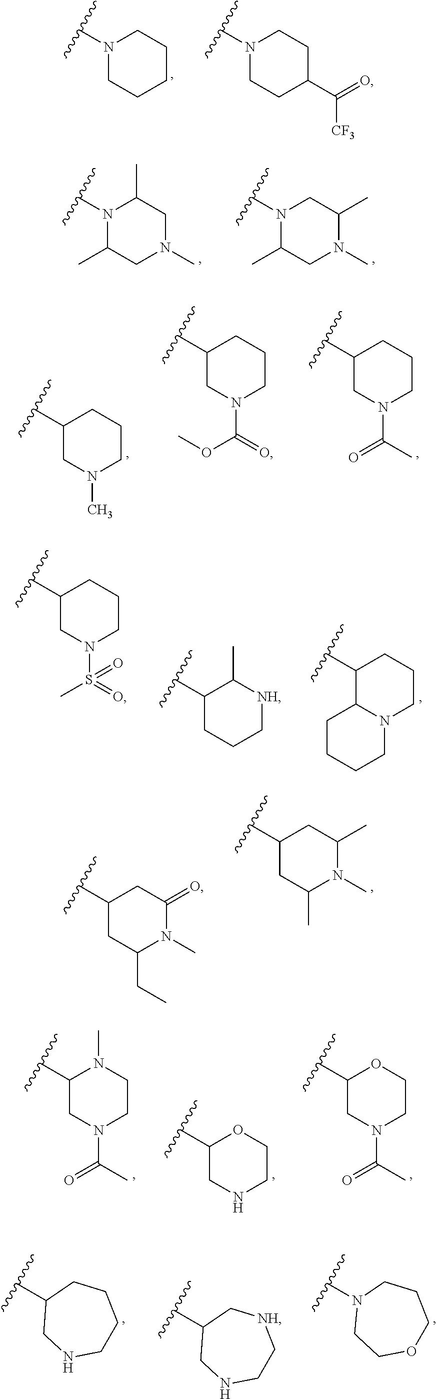

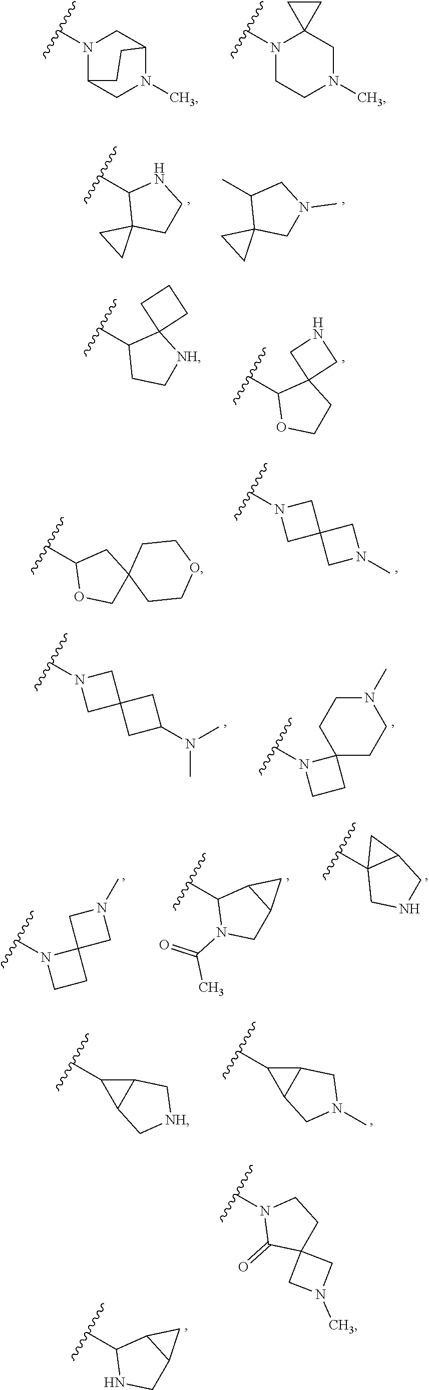

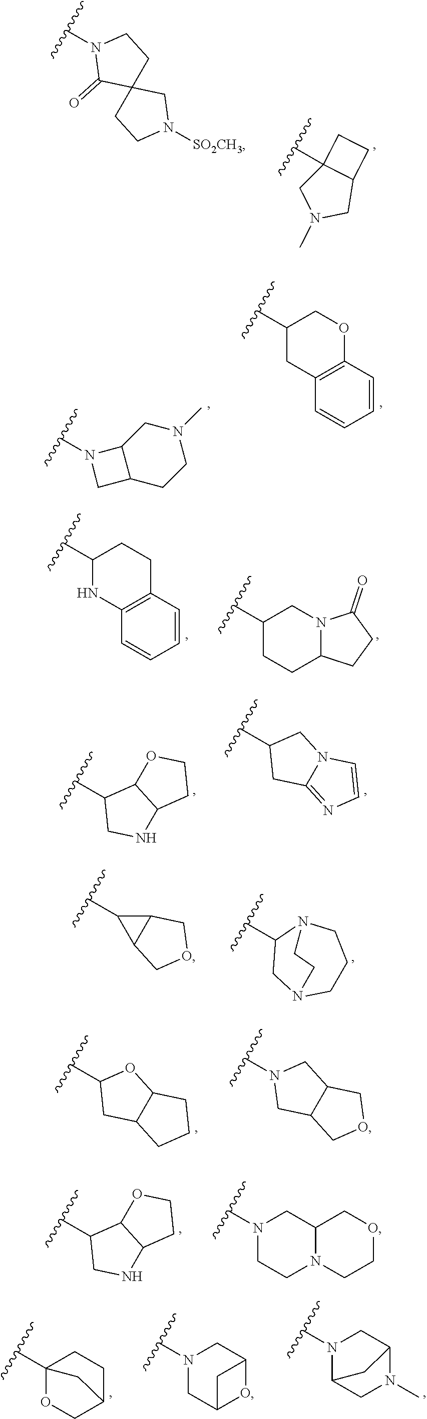

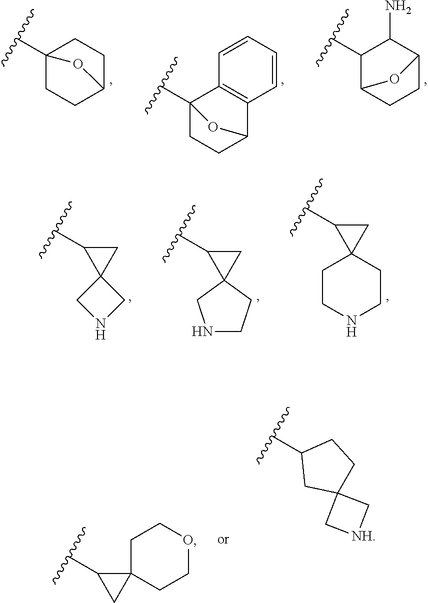

In some embodiments, W is selected from the following, or a stereoisomer thereof:

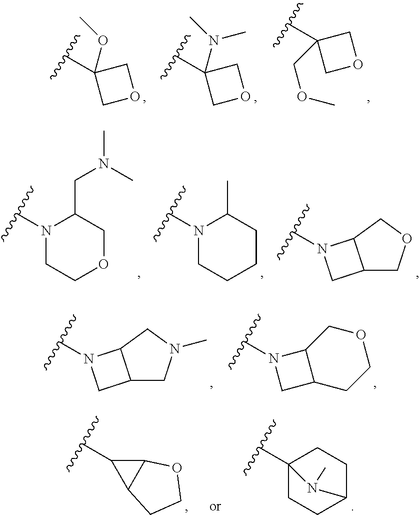

In some embodiments of any aspect described herein, W is optionally substituted 3 to 10-membered cycloalkyl. In some embodiments, W is selected from the following, or a stereoisomer thereof:

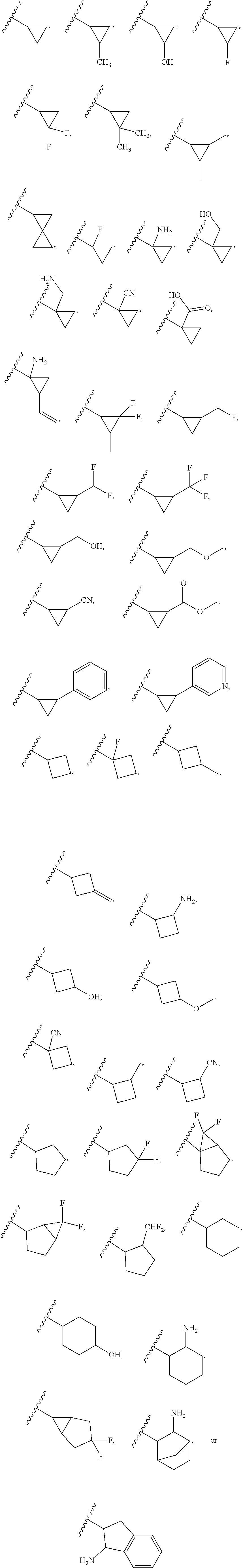

In some embodiments, W is selected from the following, or a stereoisomer thereof:

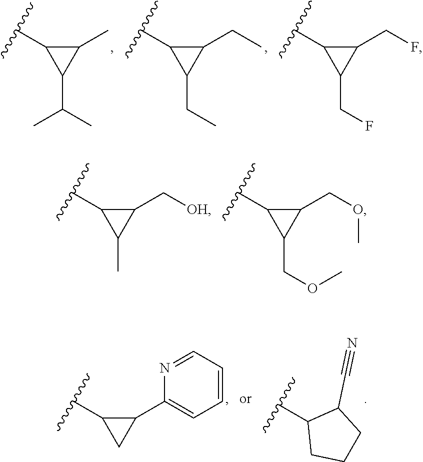

In some embodiments of any aspect described herein, W is optionally substituted 5 to 10-membered heteroaryl. In some embodiments, W is selected from the following, or a stereoisomer thereof:

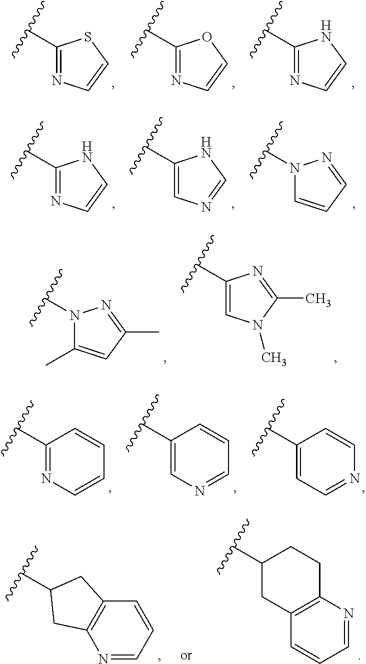

In some embodiments of any aspect described herein, W is optionally substituted 6 to 10-membered aryl. In some embodiments, W is optionally substituted phenyl.

In some embodiments of any aspect described herein, W is optionally substituted C₁-C₃ heteroalkyl. In some embodiments, W is selected from the following, or a stereoisomer thereof:

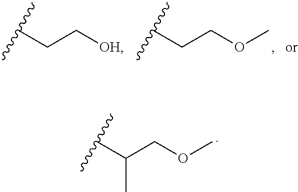

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex. # | Structure |
|---|---|
| A1 | 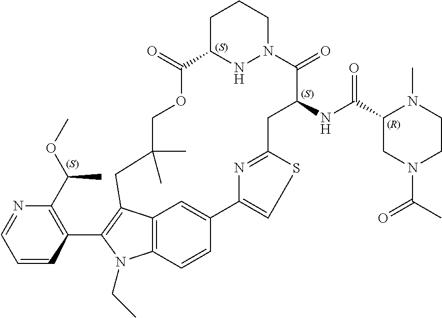 |
| A2 | 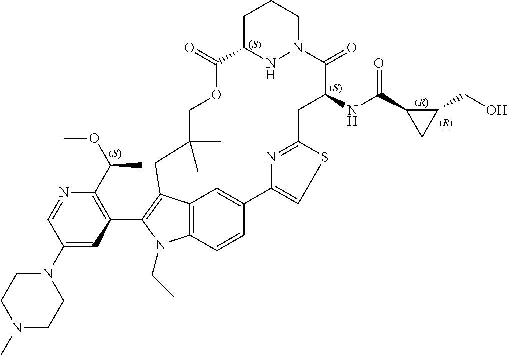 |
| A3 | 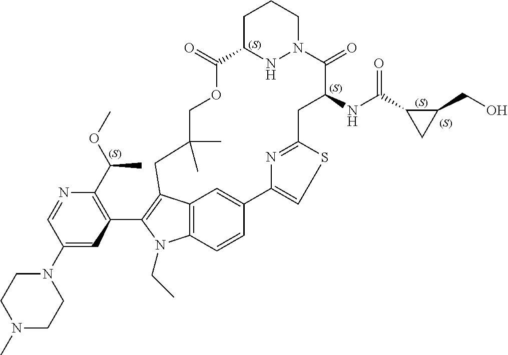 |
| A4 | 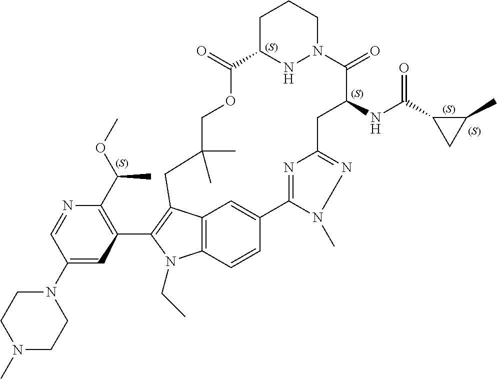 |
| A5 | 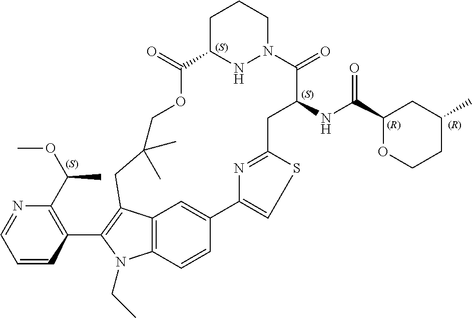 |
| A6 | 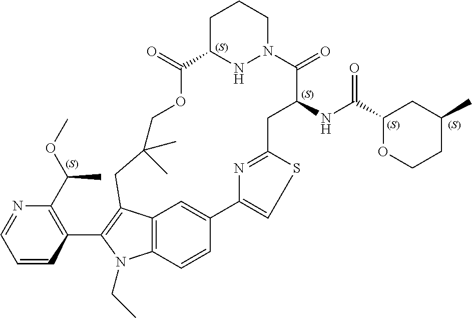 |
| A7 | 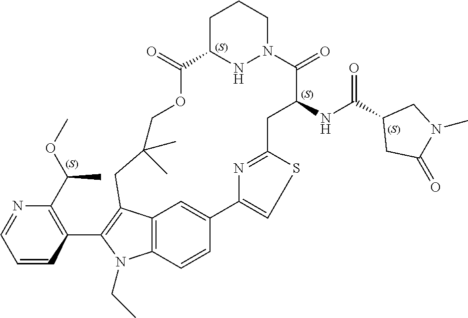 |
| A8 | 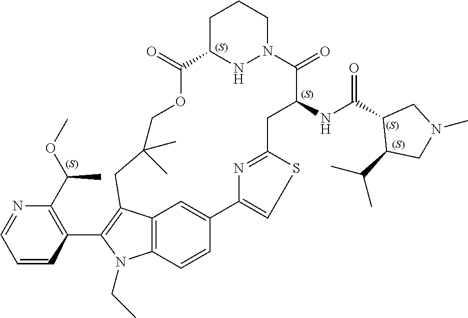 |
| A9 | 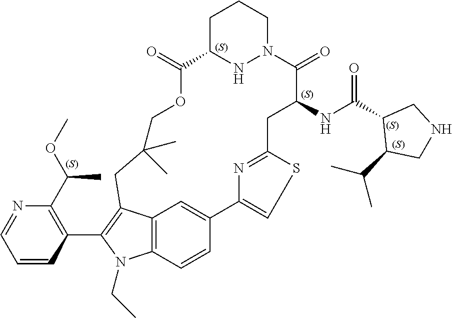 |
| A10 | 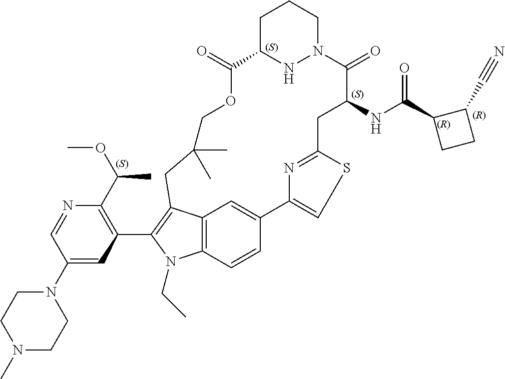 |
| A11 | 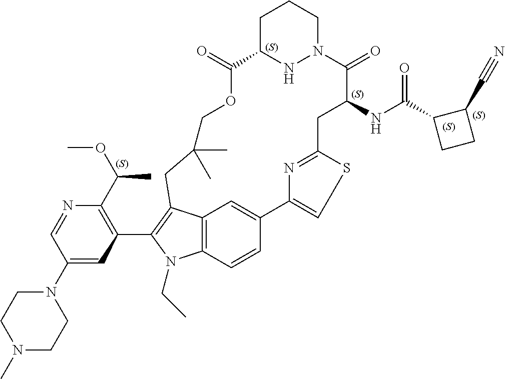 |
| A12 | 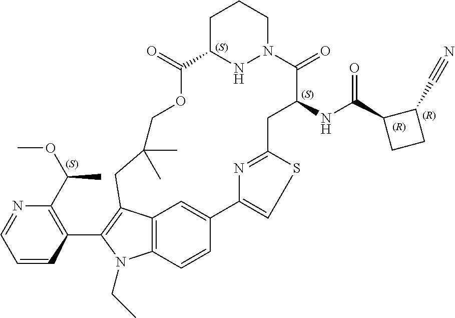 |
| A13 | 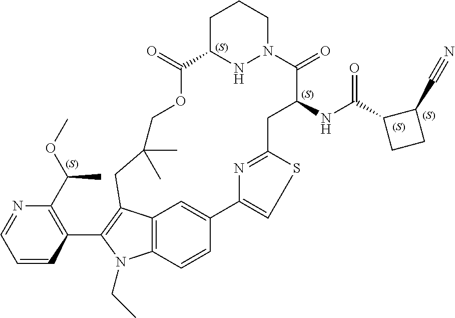 |
| A14 | 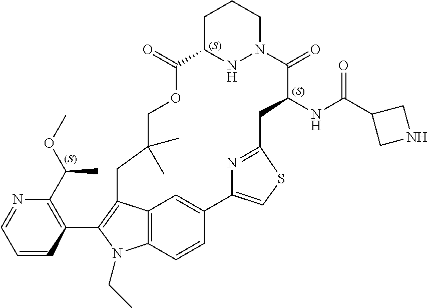 |
| A15 | 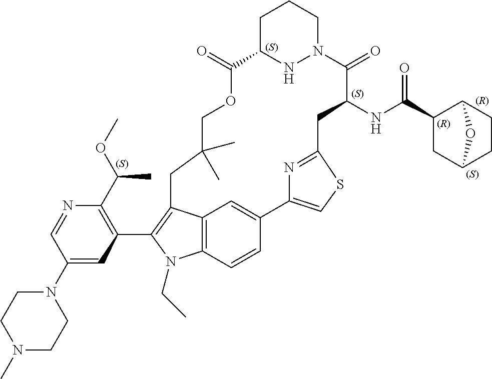 |
| A16 | 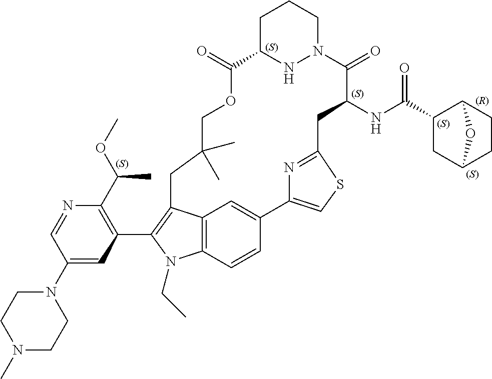 |
| A17 | 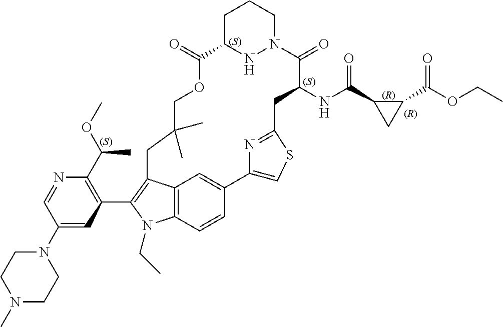 |
| A18 | 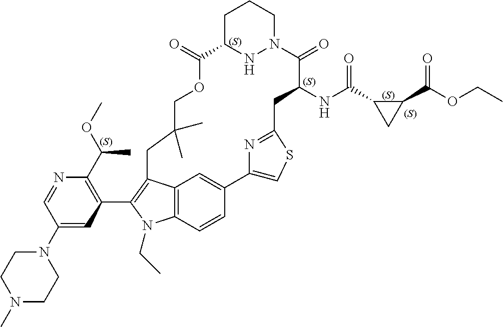 |
| A19 | 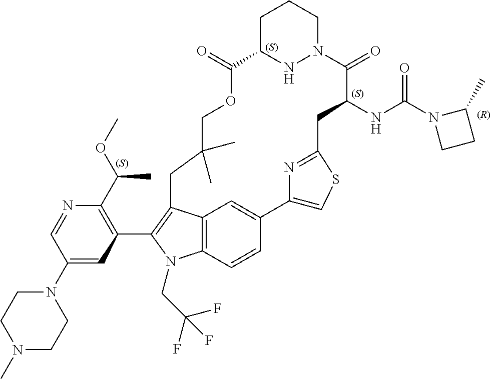 |
| A20 | 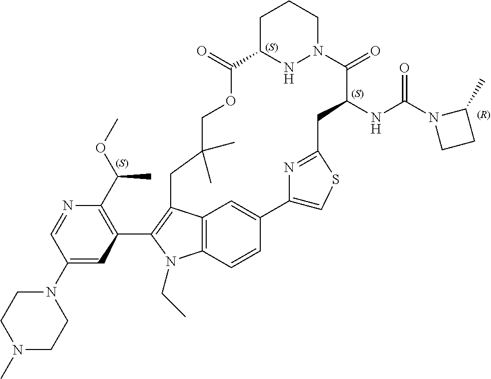 |
| A21 | 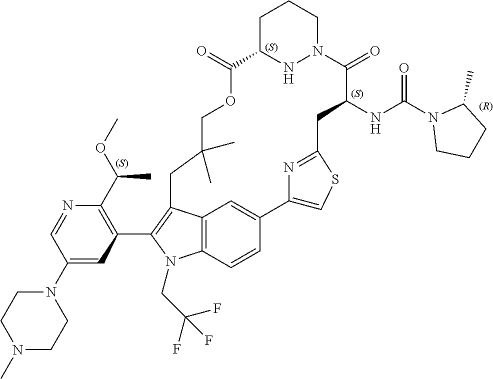 |
| A22 | 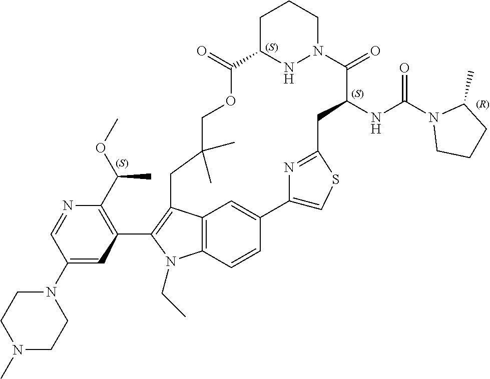 |
| A23 | 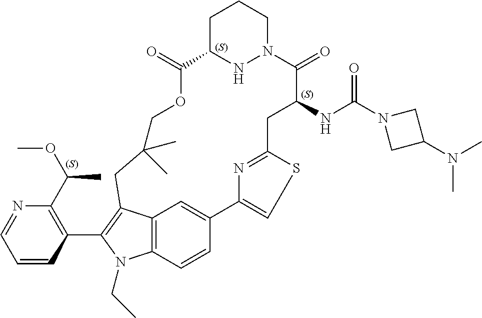 |
| A24 | 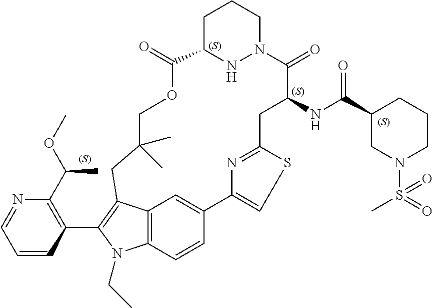 |
| A25 | 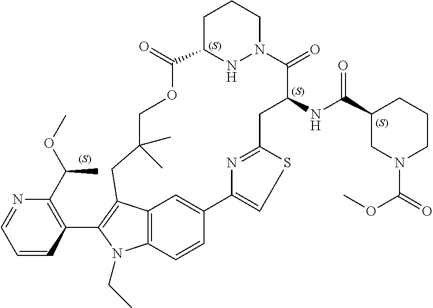 |
| A26 | 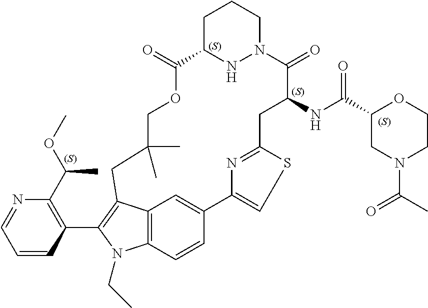 |
| A27 | 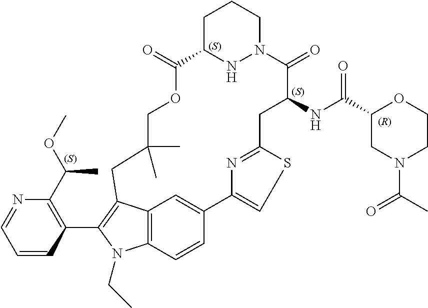 |
| A28 | 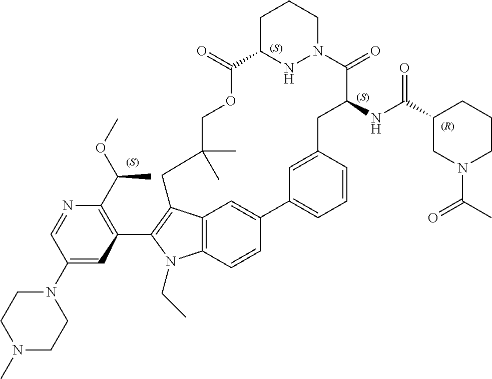 |
| A29 | 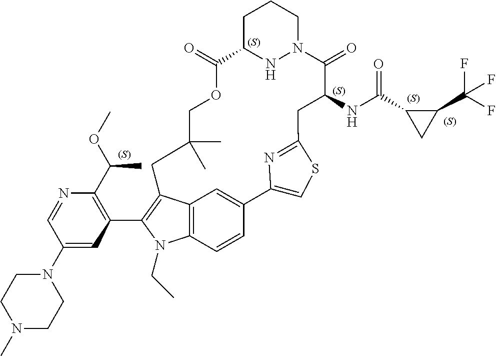 |
| A30 | 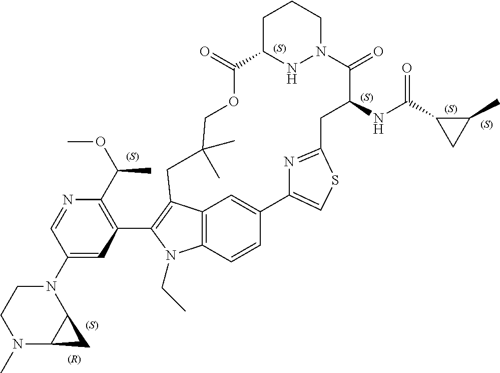 |
| A31 | 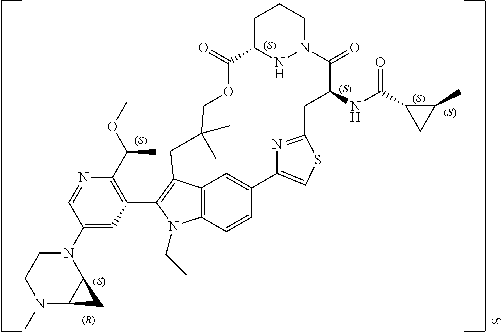 |
| A32 | 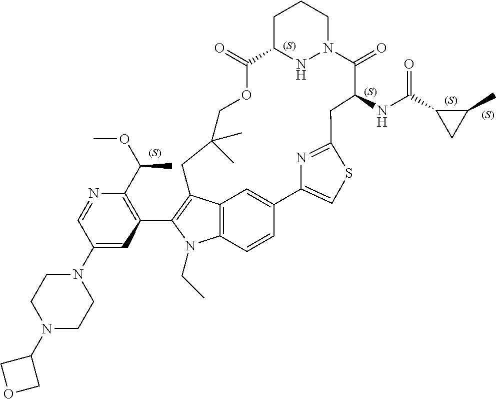 |
| A33 | 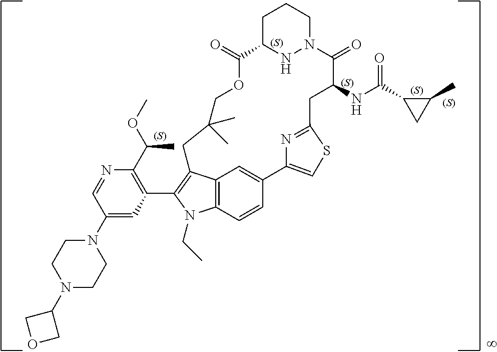 |
| A34 | 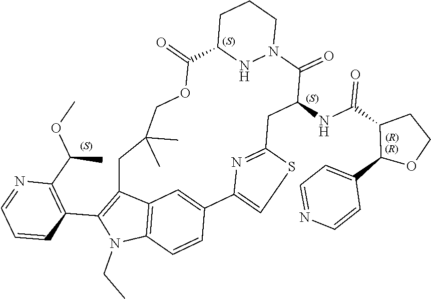 |
| A35 | 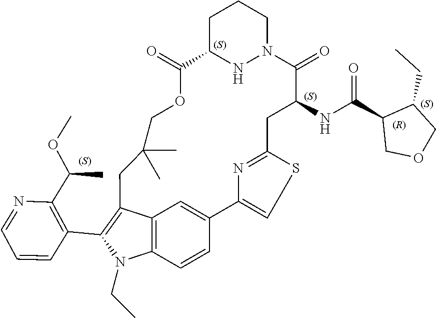 |
| A36 | 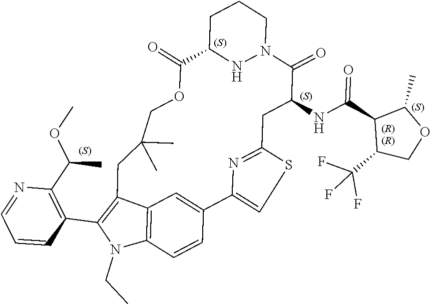 |
| A37 | 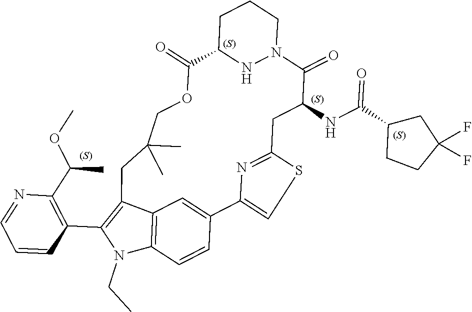 |
| A38 | 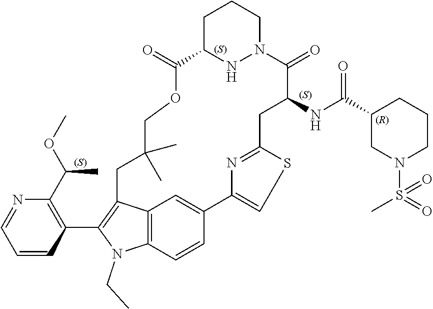 |
| A39 | 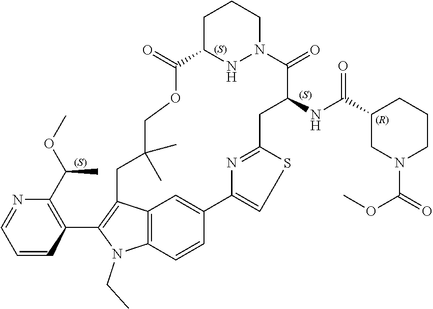 |
| A40 | 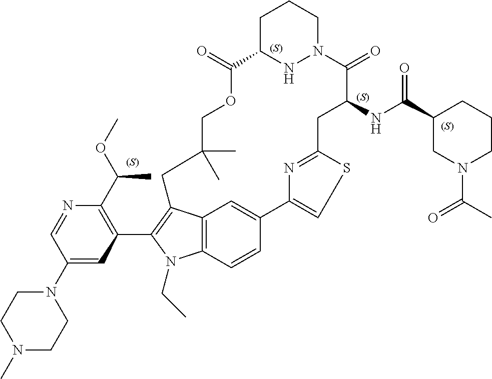 |
| A41 | 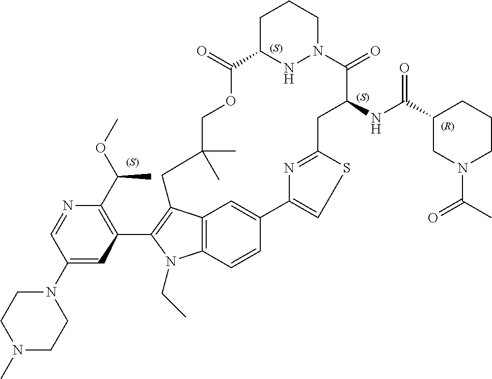 |
| A42 | 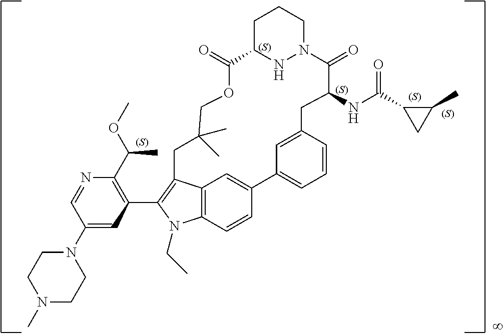 |
| A43 | 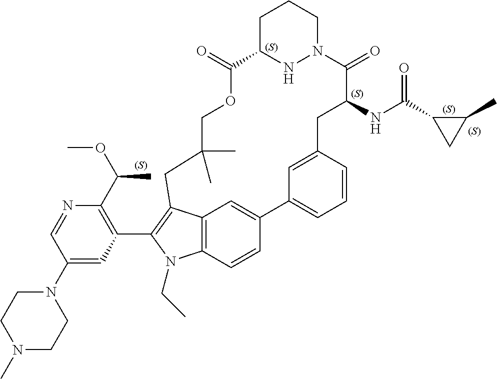 |
| A44 | 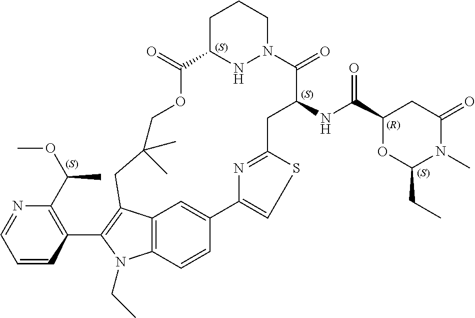 |
| A45 | 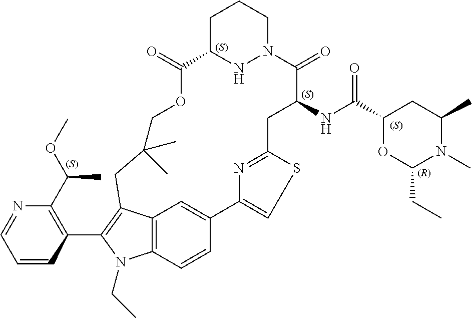 |
| A46 | 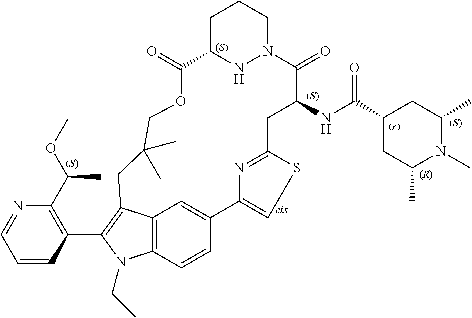 |
| A47 | 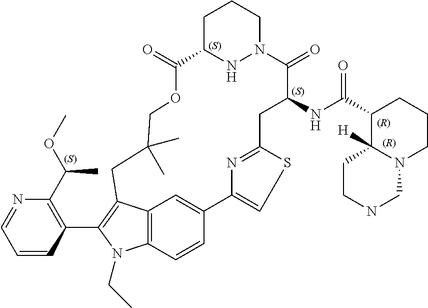 |
| A48 | 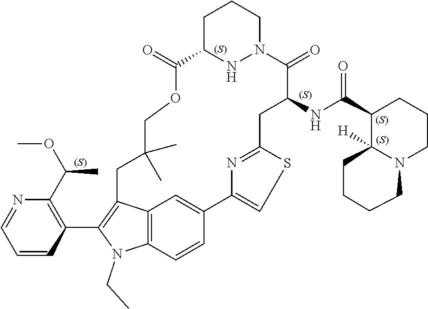 |
| A49 | 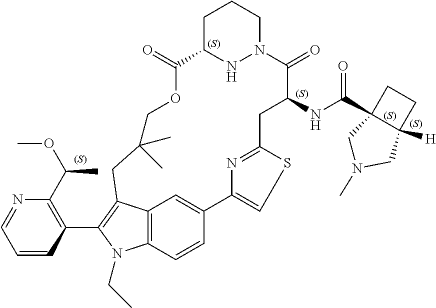 |
| A50 | 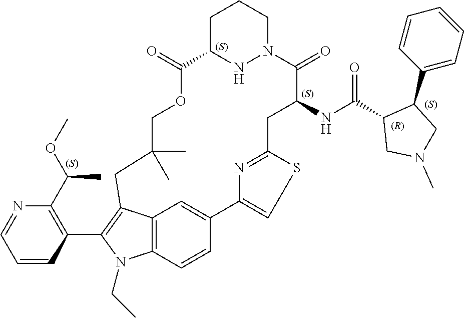 |
| A51 | 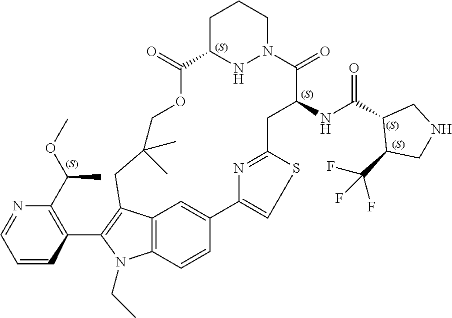 |
| A52 | 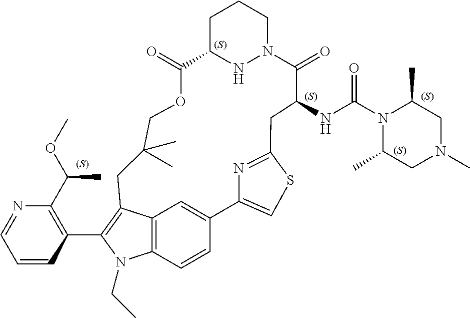 |
| A53 | 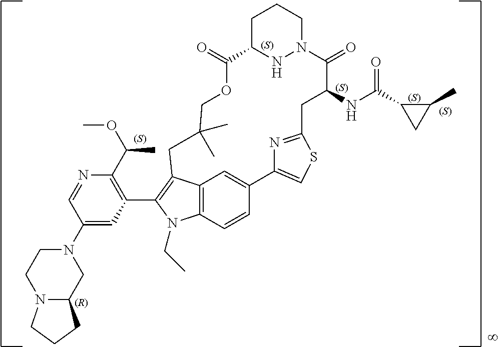 |
| A54 | 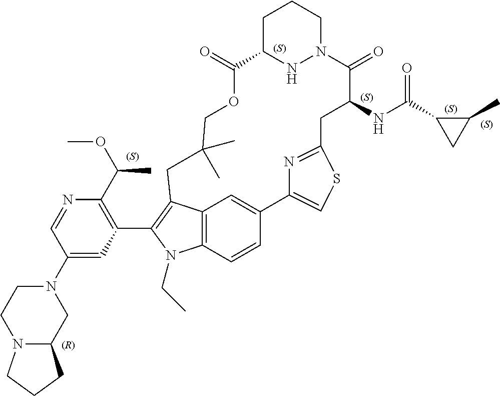 |
| A55 | 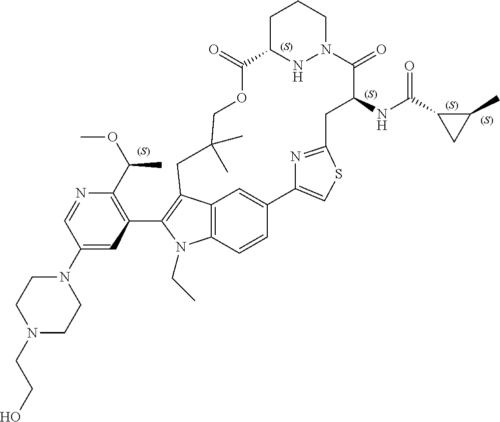 |
| A56 | 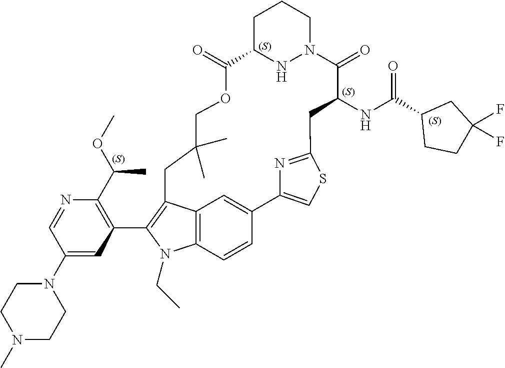 |
| A57 | 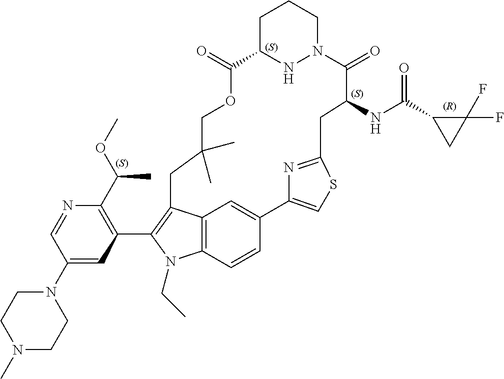 |
| A58 | 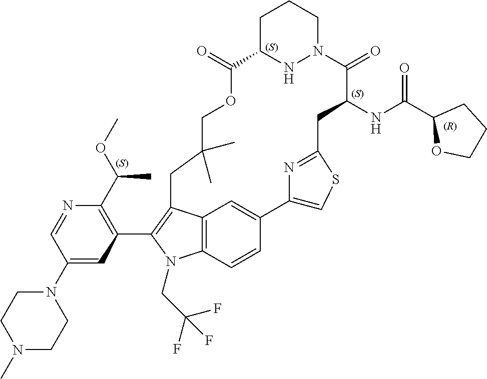 |
| A59 | 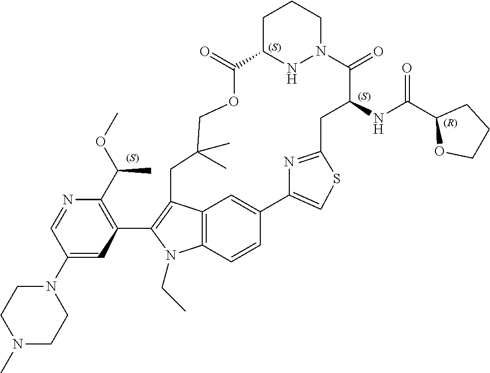 |
| A60 | 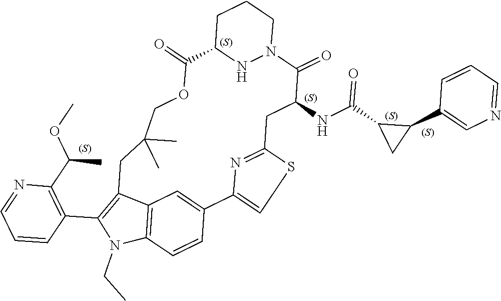 |
| A61 | 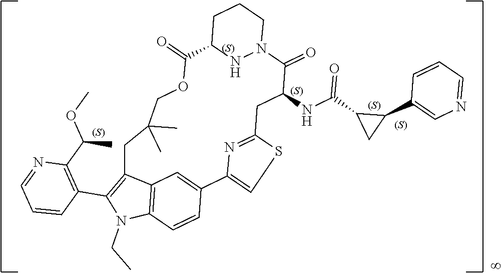 |
| A62 | 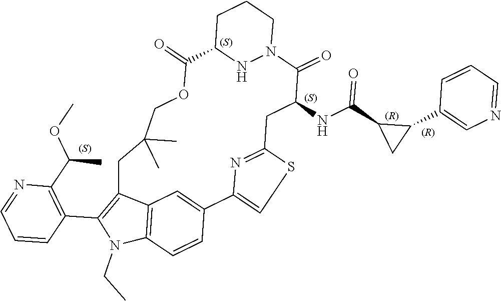 |
| A63 | 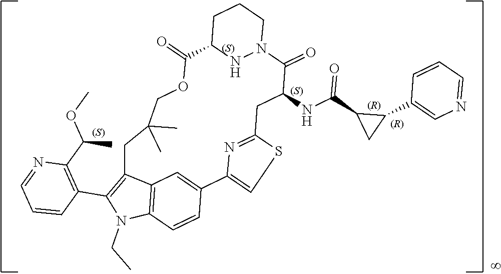 |
| A64 | 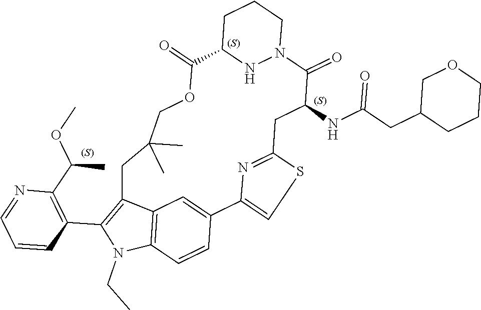 |
| A65 | 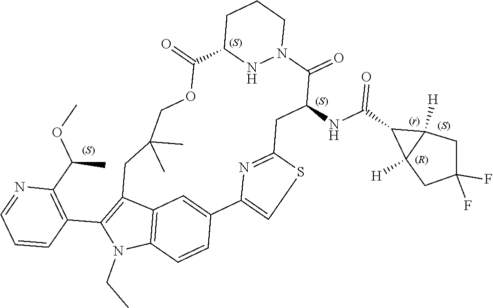 |
| A66 | 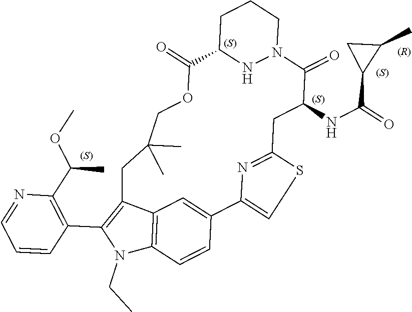 |
| A67 | 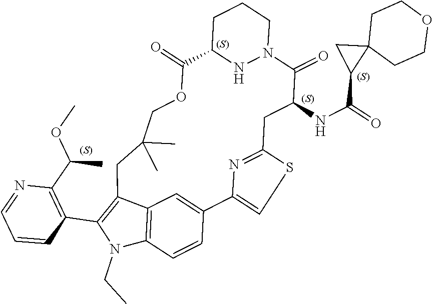 |
| A68 | 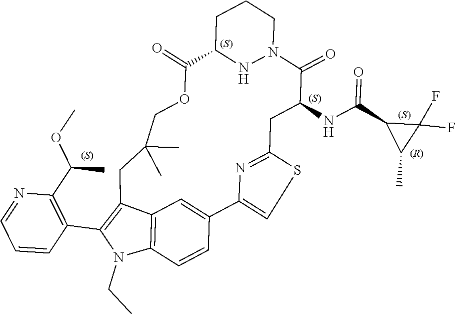 |
| A69 | 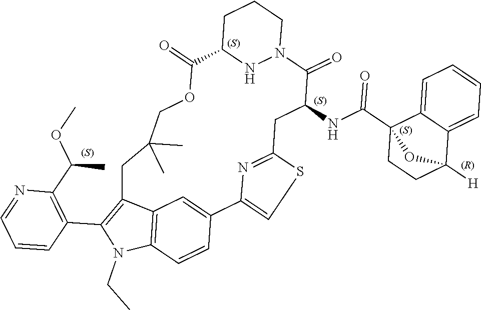 |
| A70 | 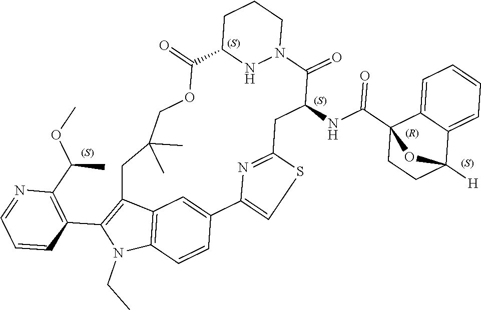 |
| A71 | 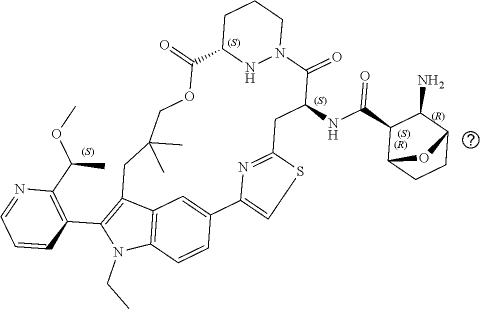 |
| A72 | 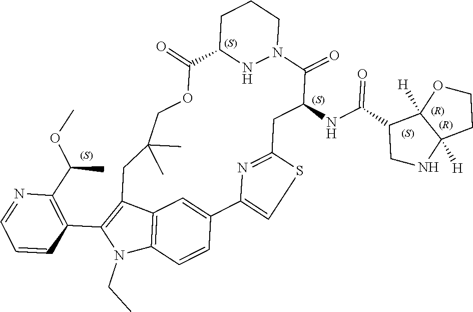 |
| A73 | 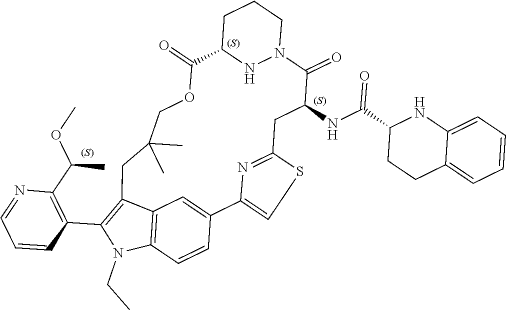 |
| A74 | 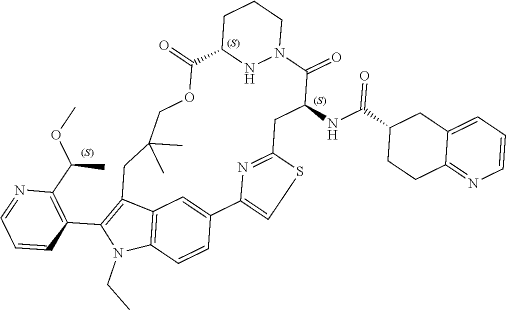 |
| A75 | 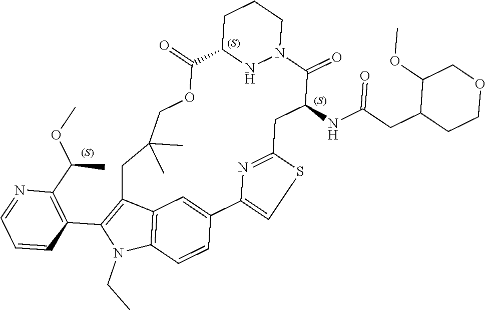 |
| A76 | 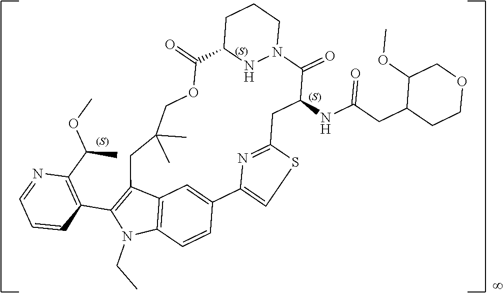 |
| A77 | 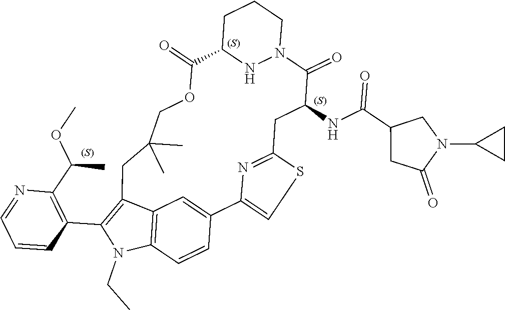 |
| A78 | 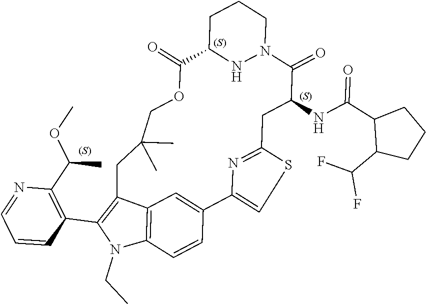 |
| A79 | 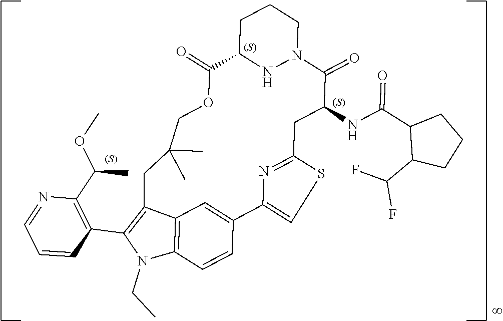 |
| A80 | 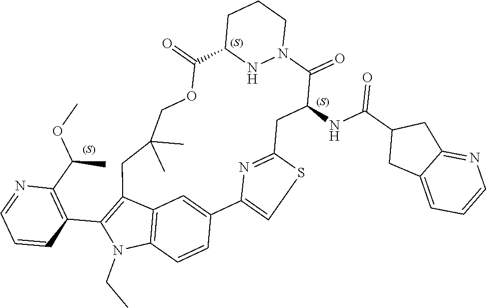 |
| A81 | 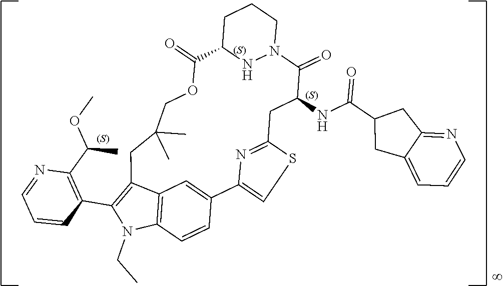 |
| A82 | 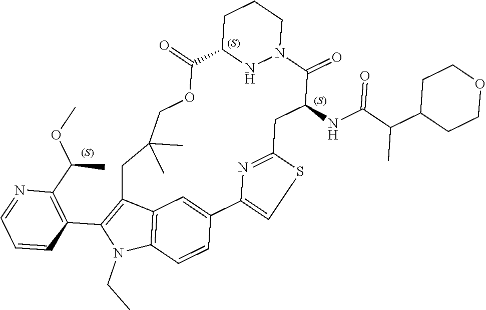 |
| A83 | 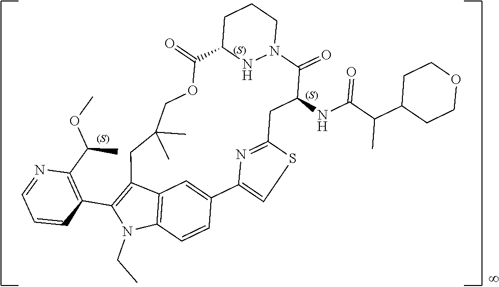 |
| A84 | 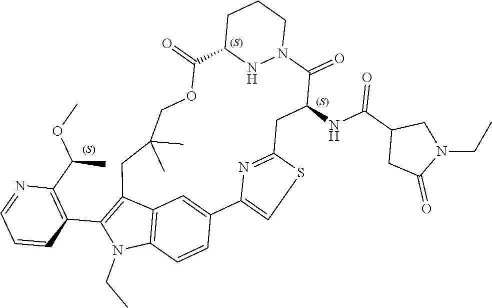 |
| A85 | 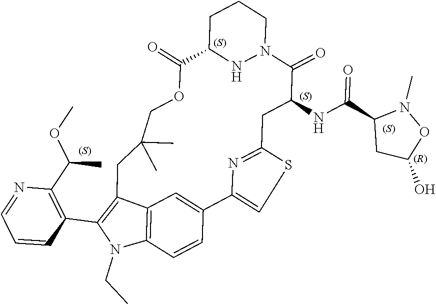 |
| A86 | 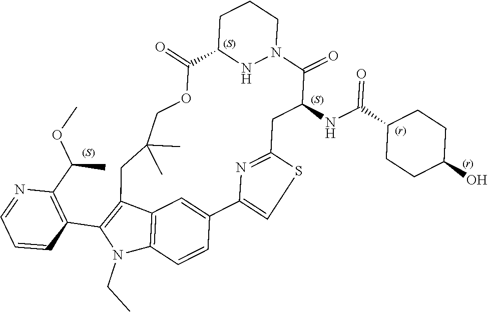 |
| A87 | 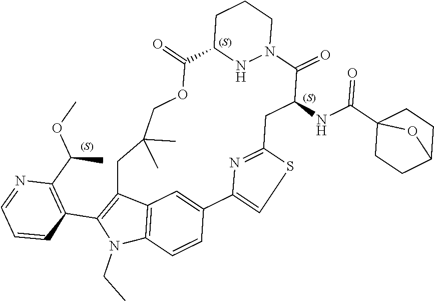 |
| A88 | 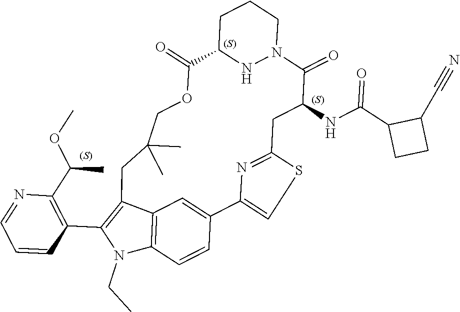 |
| A89 | 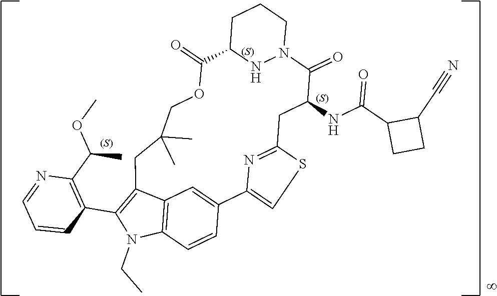 |
| A90 | 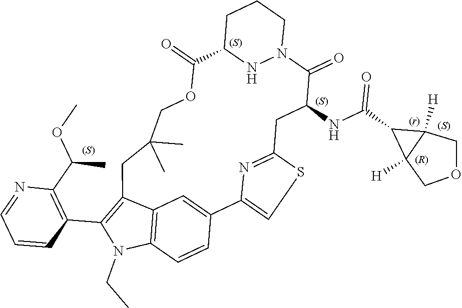 |
| A91 | 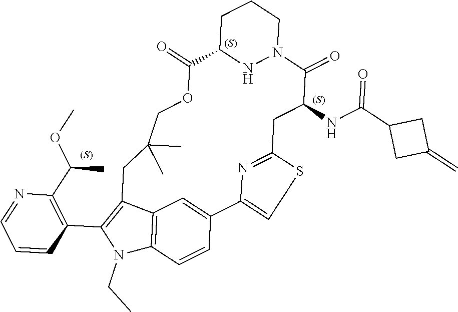 |
| A92 | 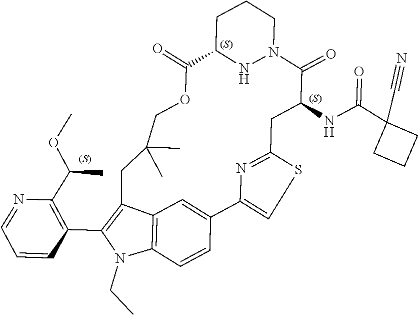 |
| A93 | 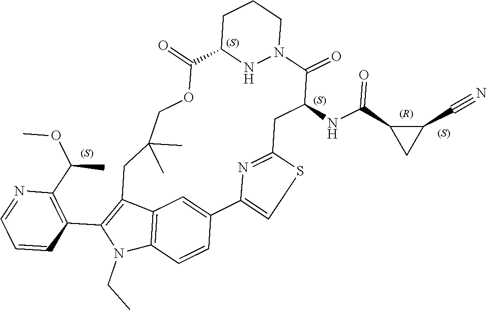 |
| A94 | 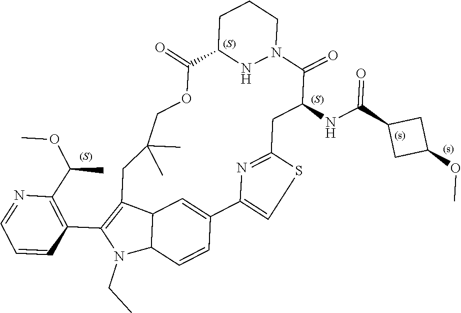 |
| A95 | 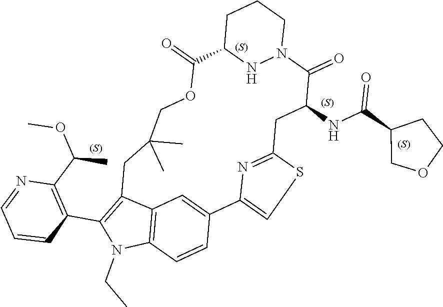 |
| A96 | 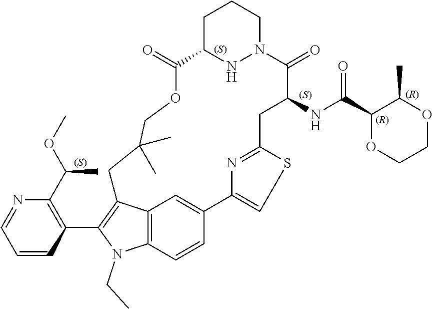 |
| A97 | 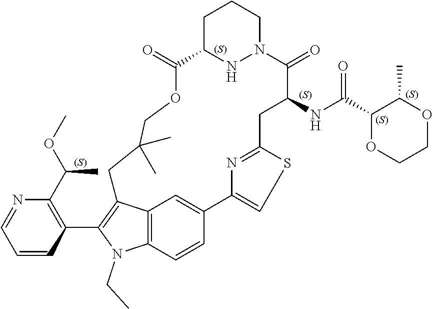 |
| A98 | 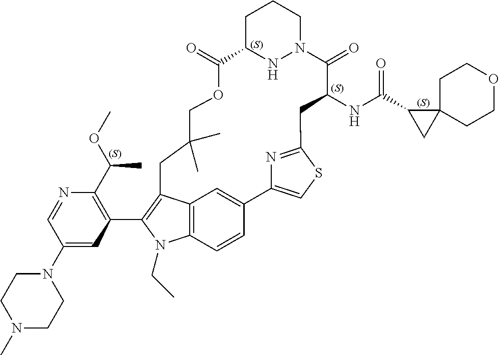 |
| A99 | 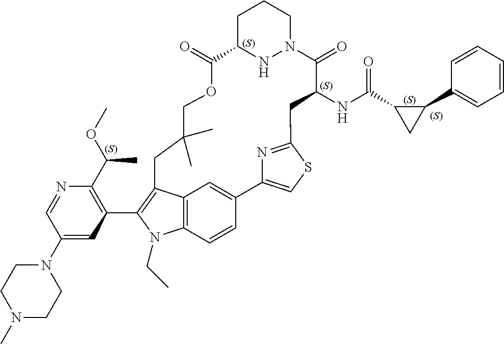 |
| A100 | 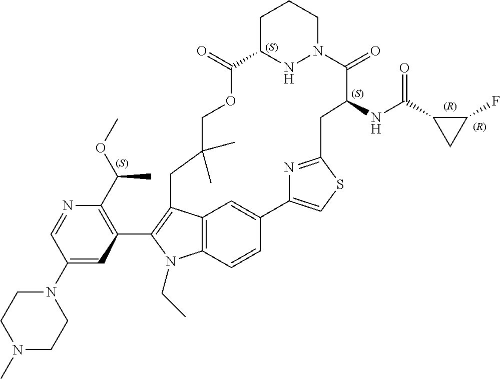 |
| A101 | 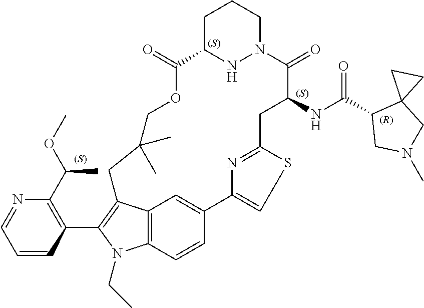 |
| A102 | 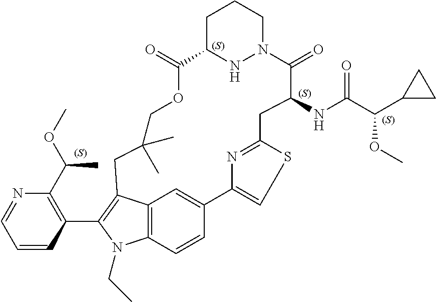 |
| A103 | 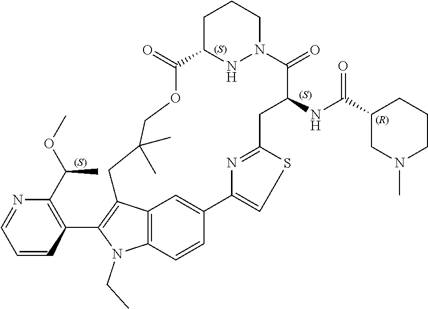 |
| A104 | 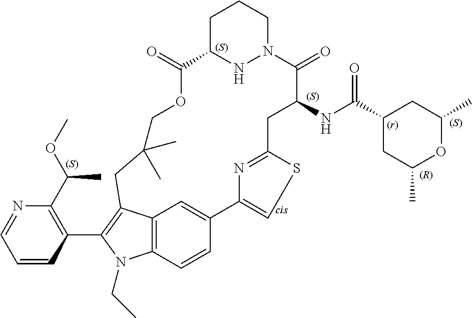 |
| A105 | 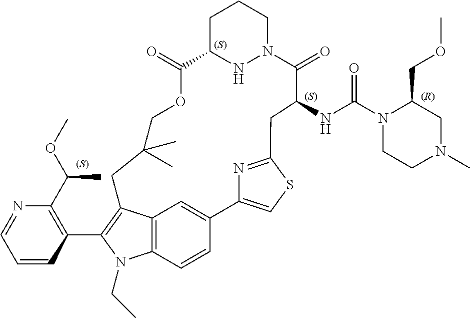 |
| A106 | 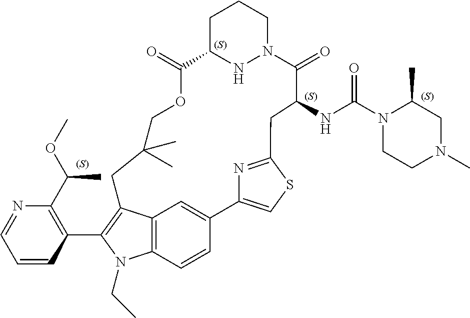 |
| A107 | 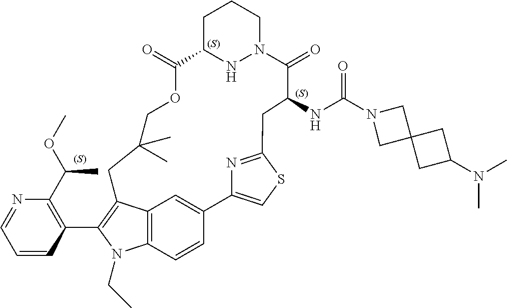 |
| A108 | 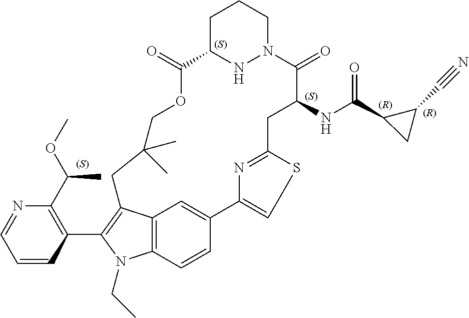 |
| A109 | 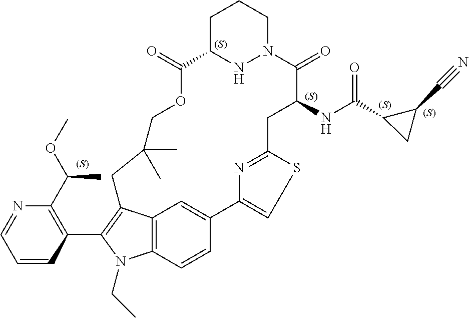 |
| A110 | 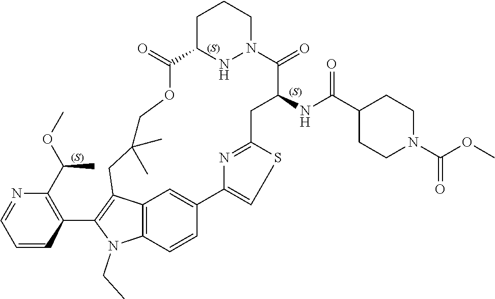 |
| A111 | 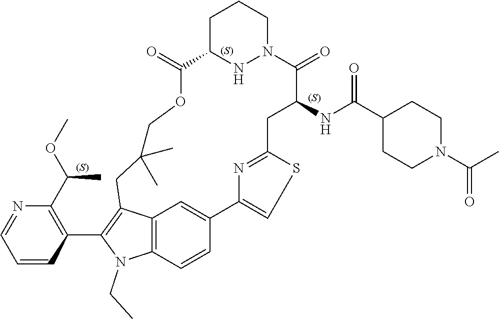 |
| A112 | 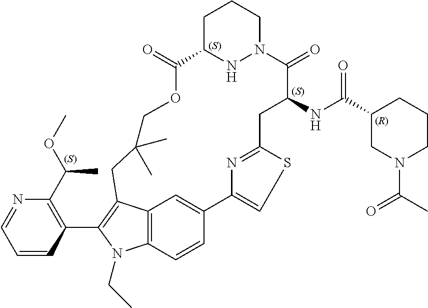 |
| A113 | 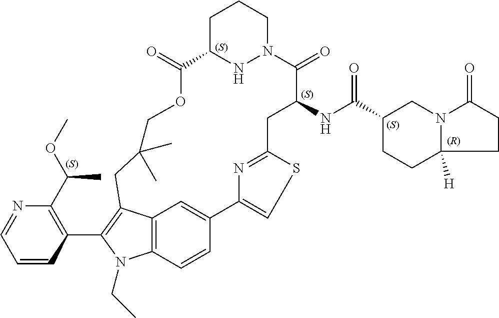 |
| A114 | 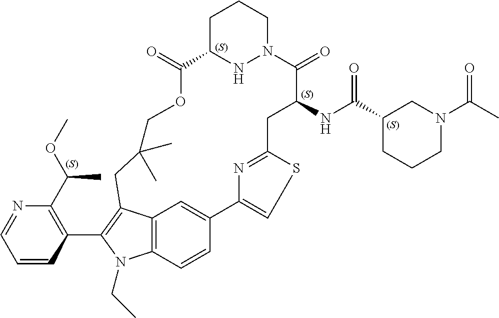 |
| A115 | 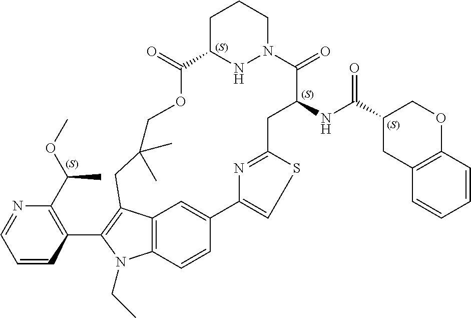 |
| A116 | 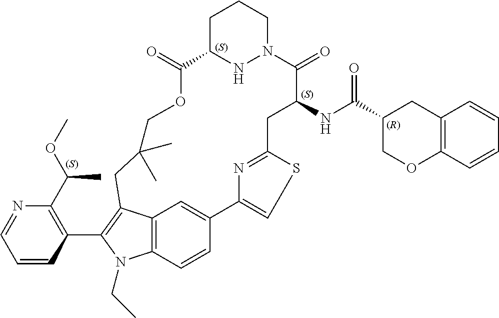 |
| A117 | 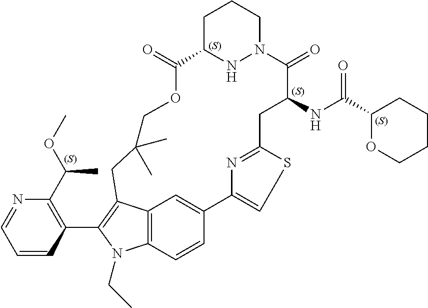 |
| A118 | 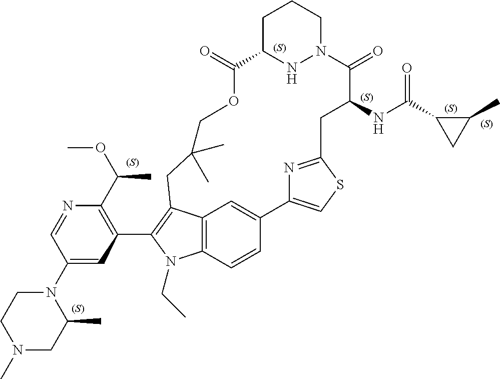 |
| A119 | 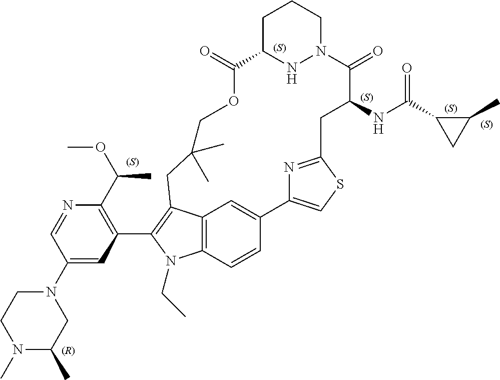 |
| A120 | 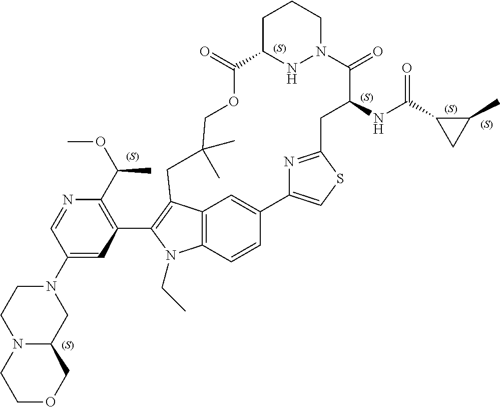 |
| A121 | 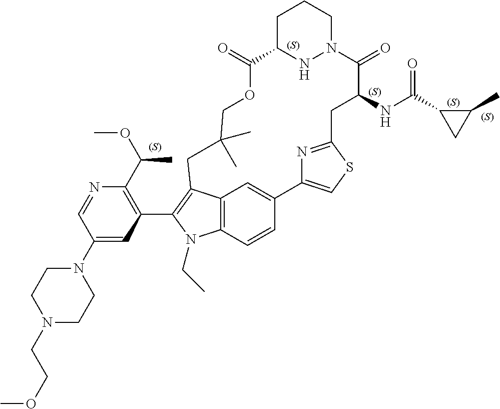 |
| A122 | 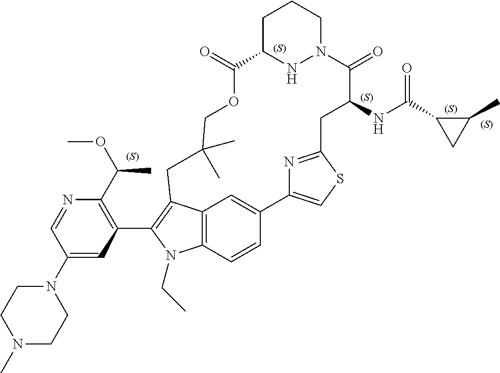 |
| A123 | 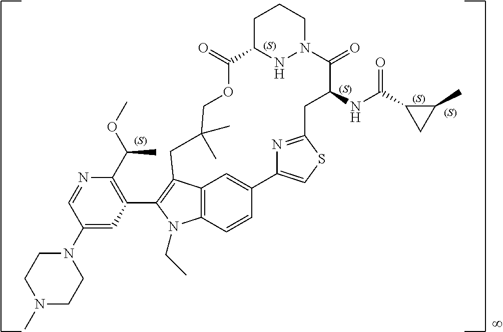 |
| A124 | 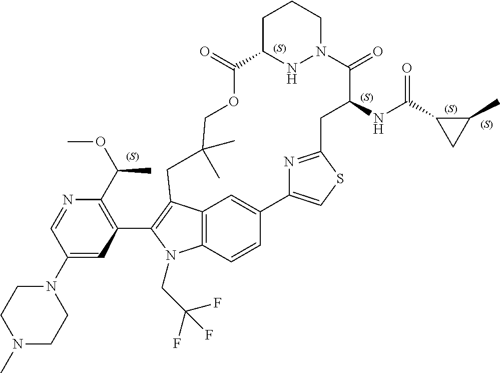 |
| A125 | 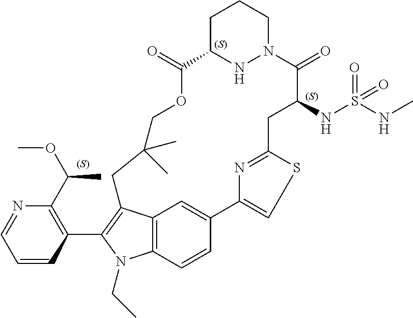 |
| A126 | 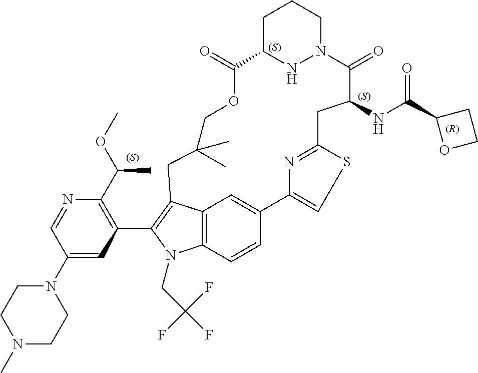 |
| A127 | 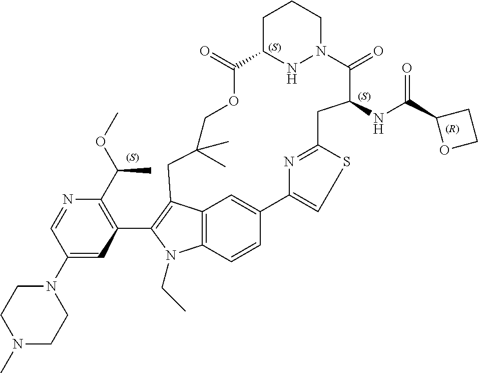 |
| A128 | 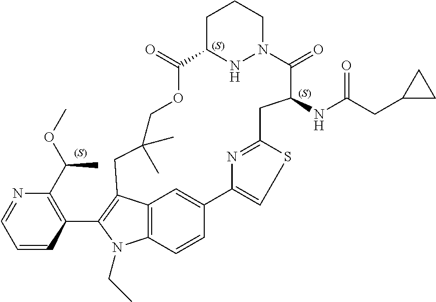 |
| A129 | 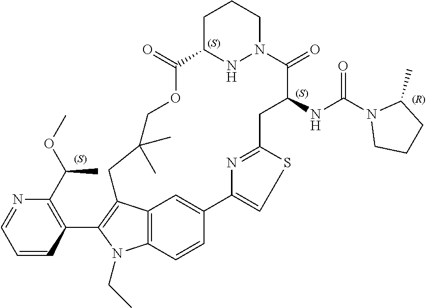 |
| A130 | 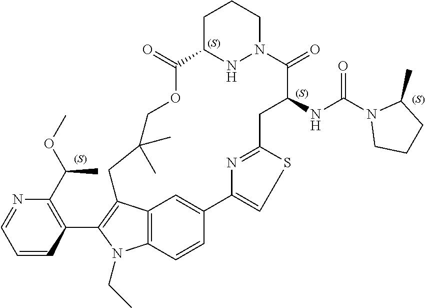 |
| A131 | 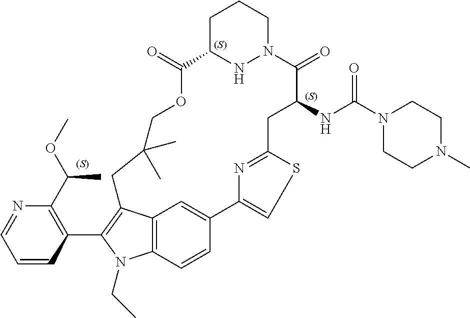 |
| A132 | 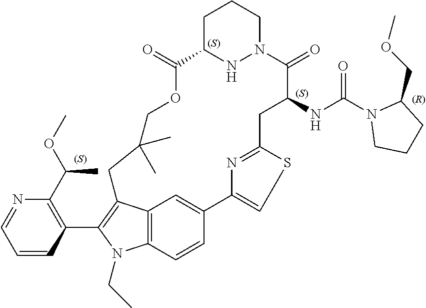 |
| A133 | 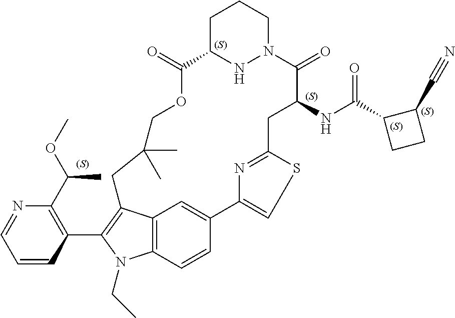 |
| A134 | 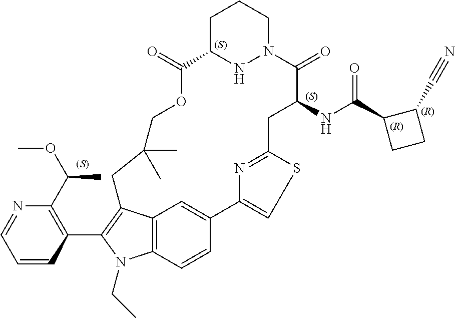 |
| A135 | 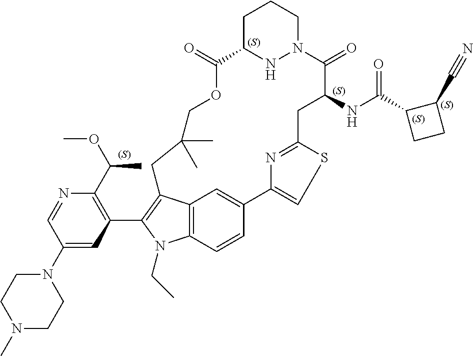 |
| A136 | 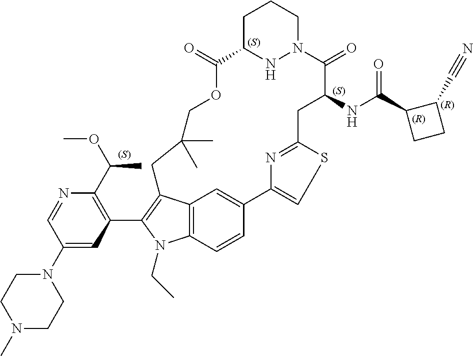 |
| A137 | 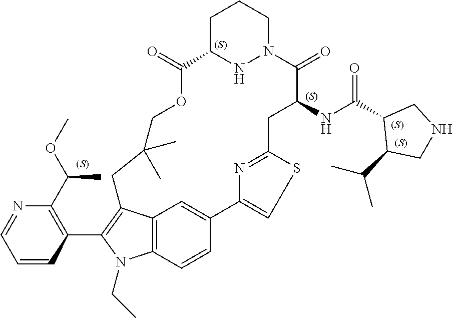 |
| A138 | 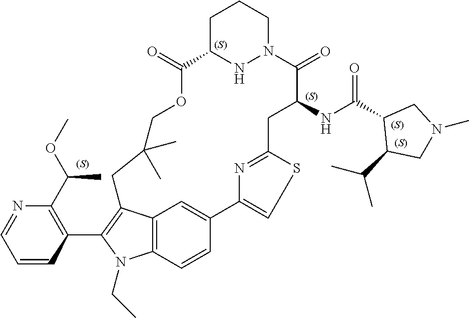 |
| A139 | 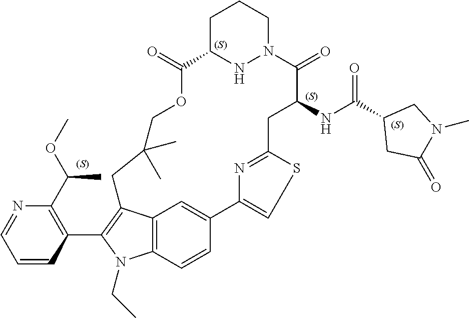 |
| A140 | 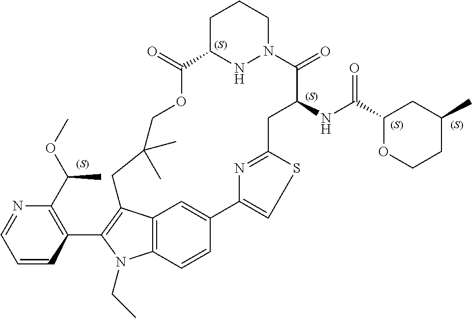 |
| A141 | 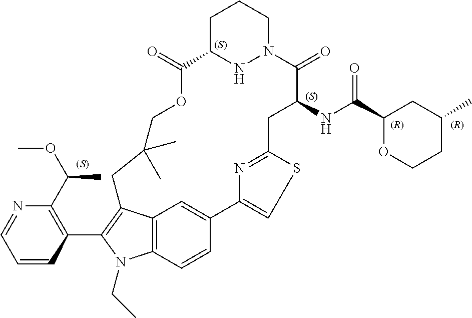 |
| A142 | 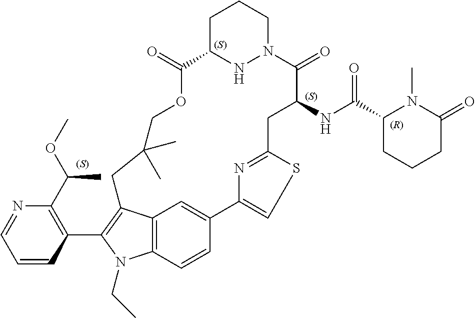 |
| A143 | 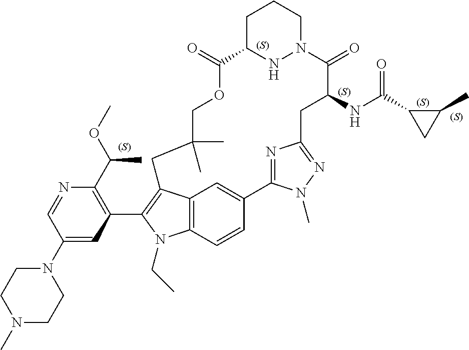 |
| A144 | 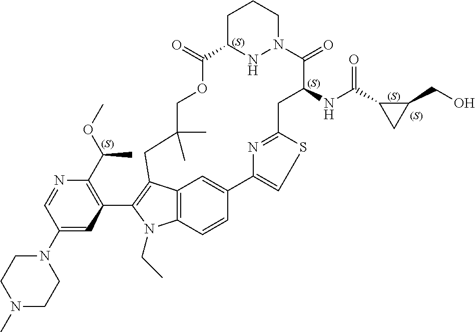 |
| A145 | 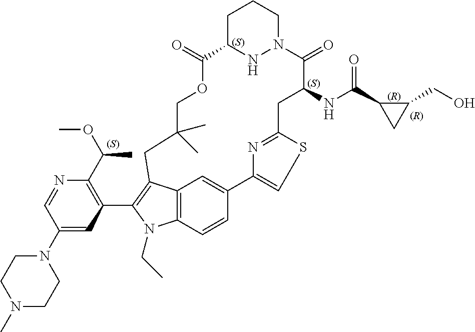 |
| A146 | 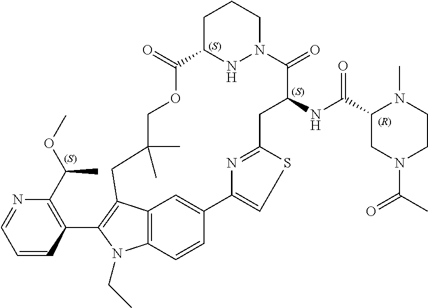 |
| A147 | 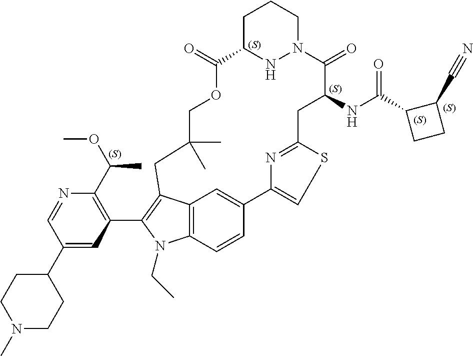 |
| A148 | 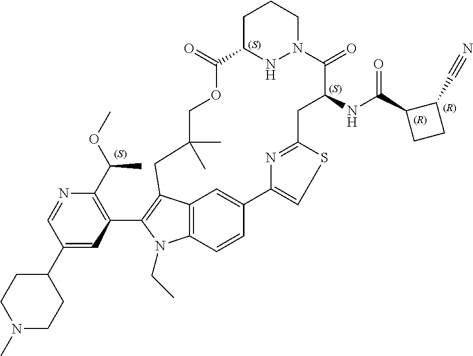 |
| A149 | 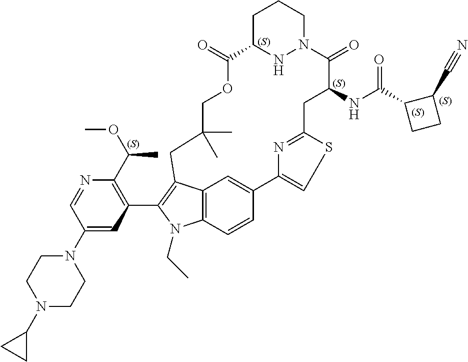 |
| A150 | 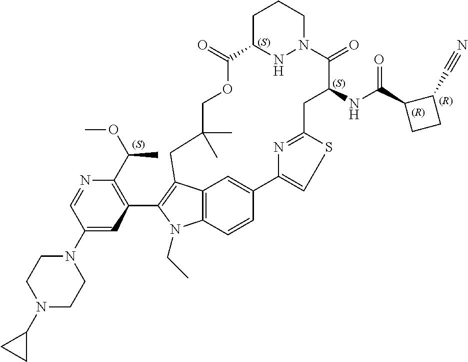 |
| A151 | 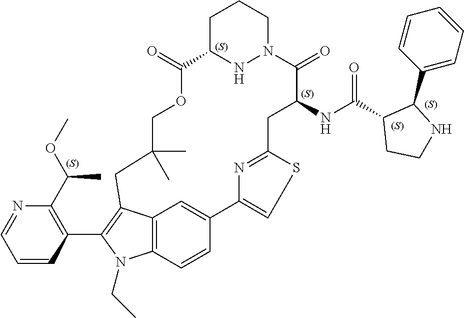 |
| A152 | 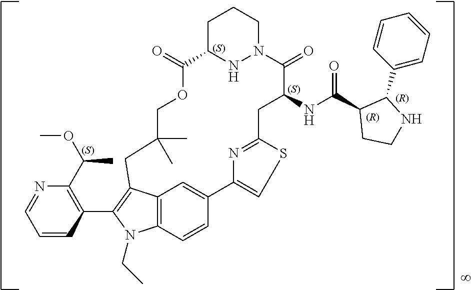 |
| A153 | 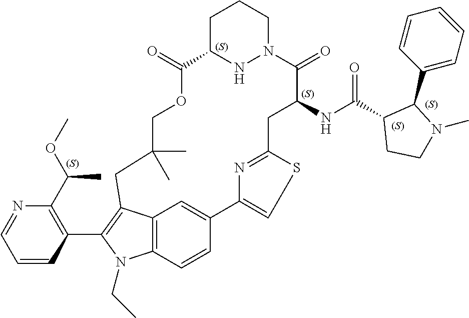 |
| A154 | 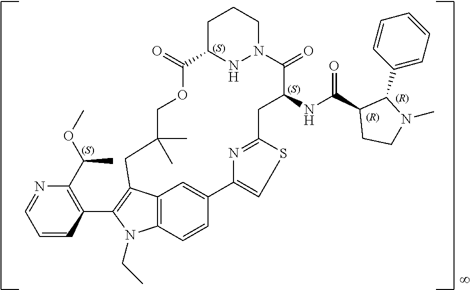 |
| A155 | 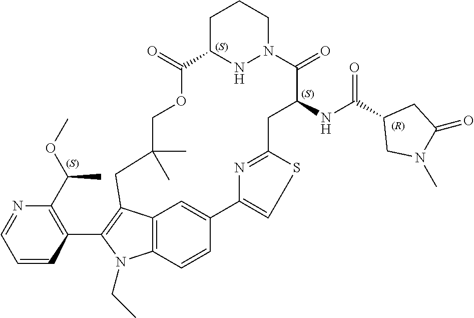 |
| A156 | 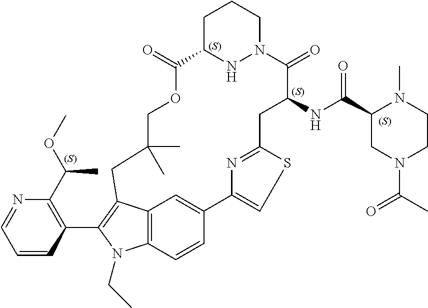 |
| A157 | 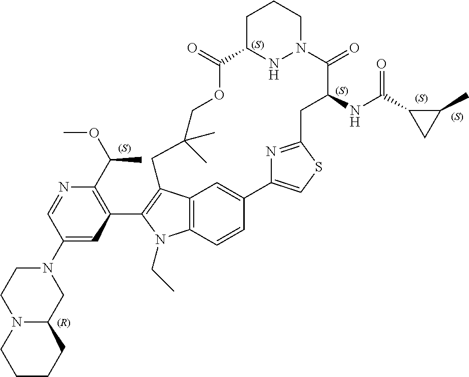 |
| A158 | 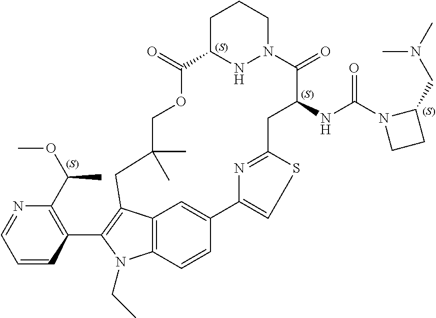 |
| A159 | 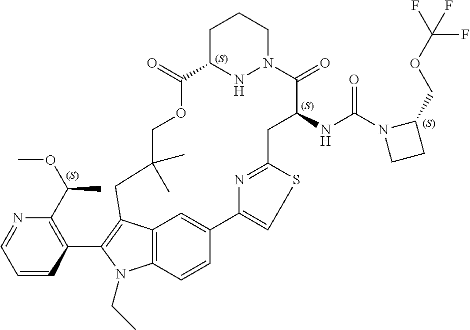 |
| A160 | 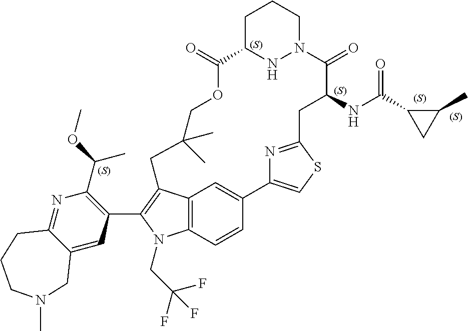 |
| A161 | 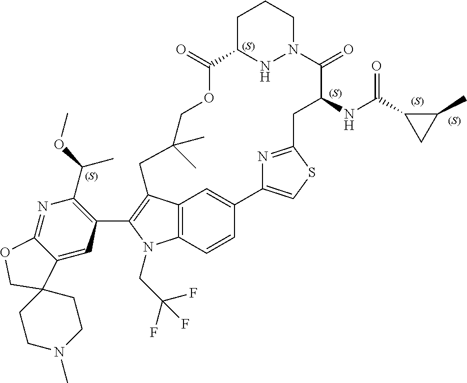 |
| A162 | 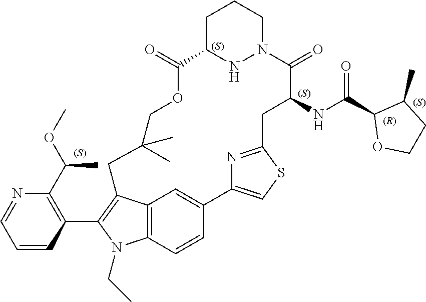 |
| A163 | 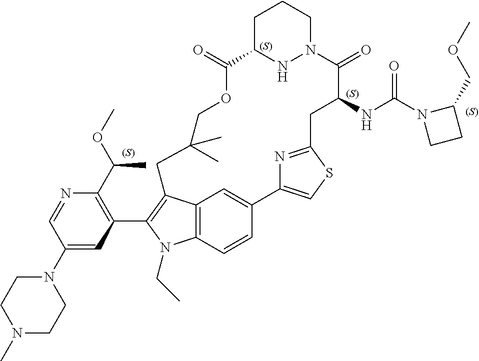 |
| A164 | 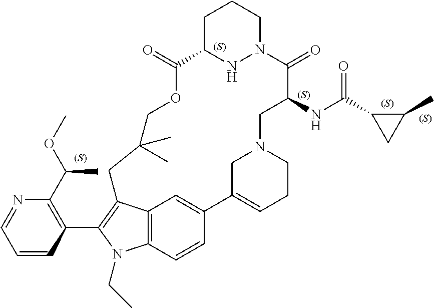 |
| A165 | 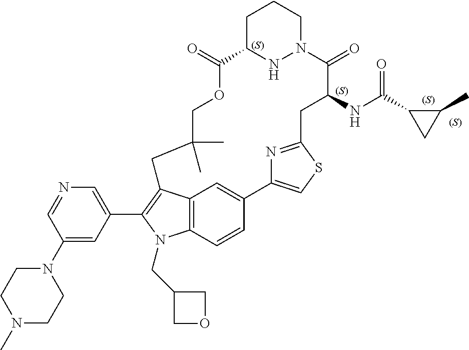 |
| A166 | 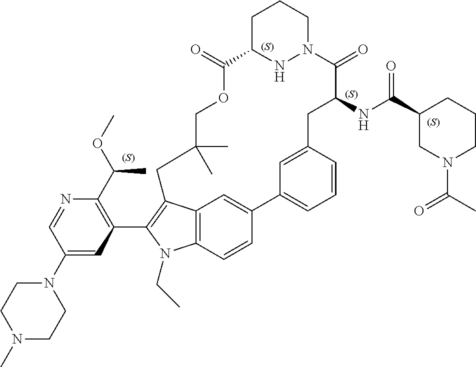 |
| A167 | 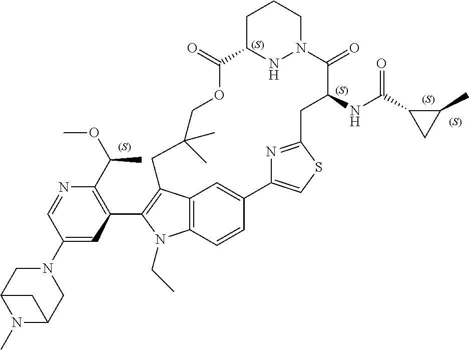 |
| A168 | 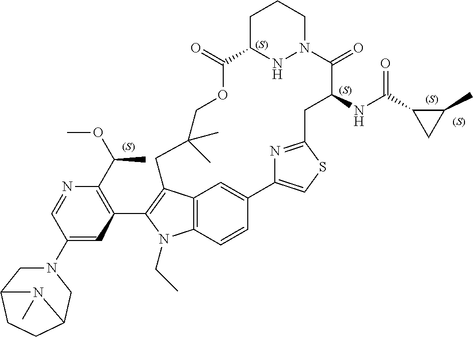 |
| A169 | 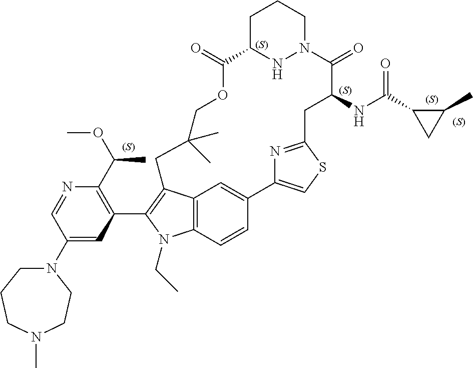 |
| A170 | 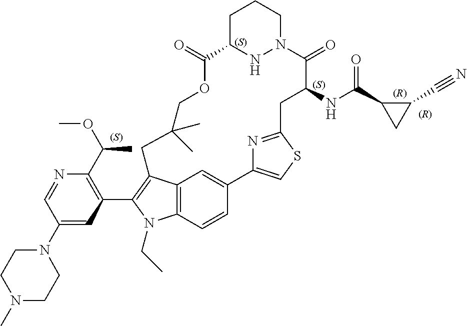 |
| A171 | 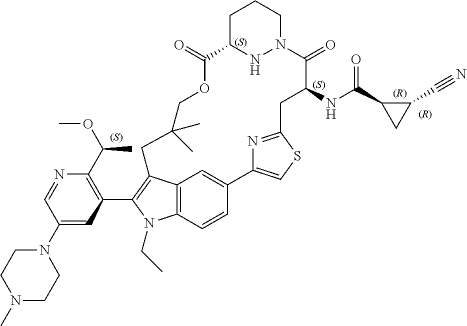 |
| A172 | 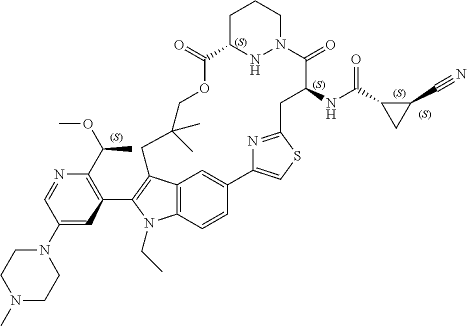 |
| A173 | 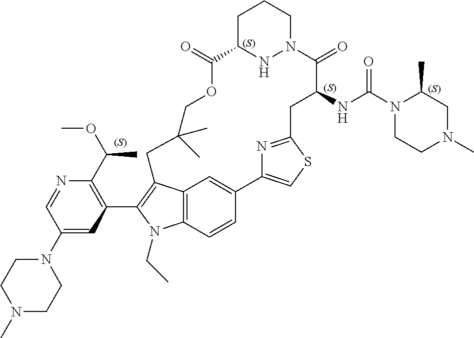 |
| A174 | 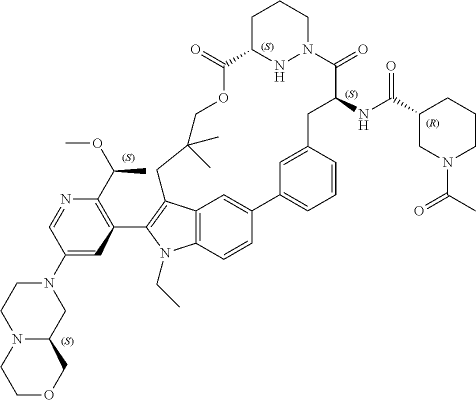 |
| A175 | 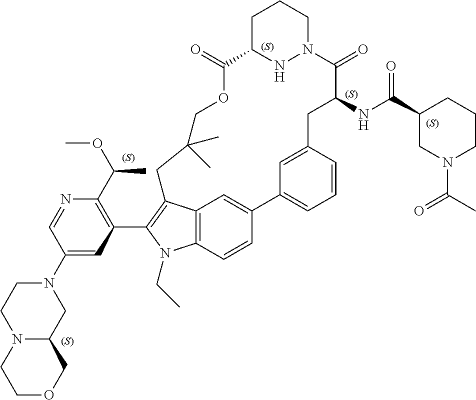 |
| A176 | 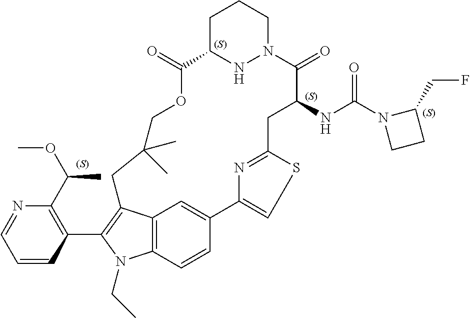 |
| A177 | 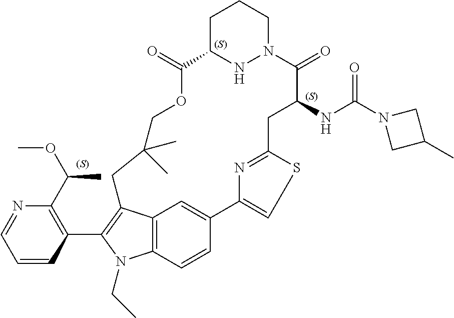 |
| A178 | 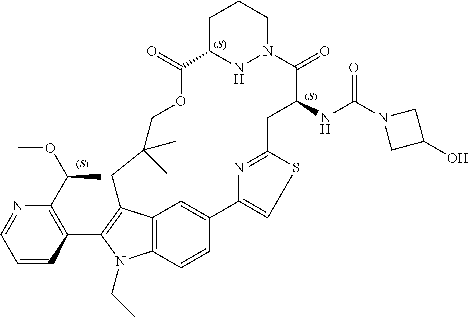 |
| A179 | 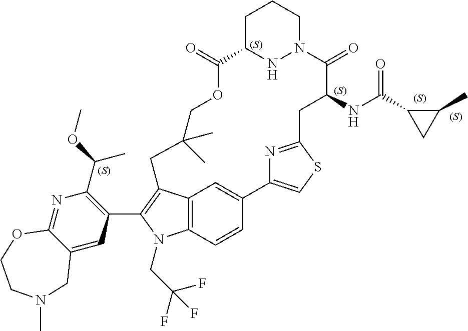 |
| A180 | 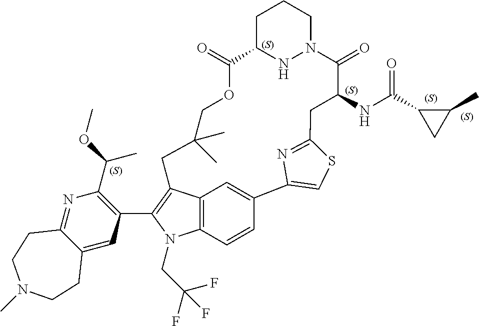 |
| A181 | 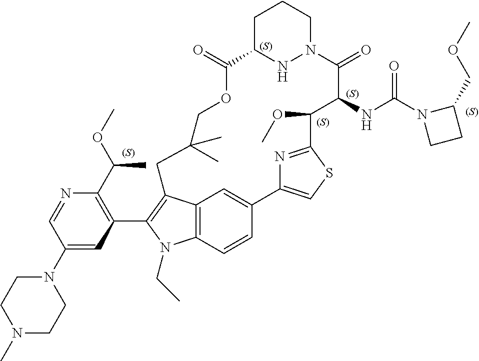 |
| A182 | 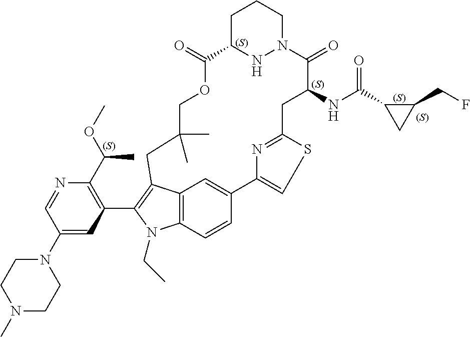 |
| A183 | 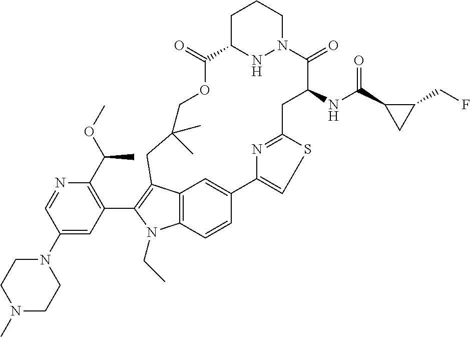 [SMILES: CCn1c(-c2cc(N3CCN(C)CC3)cnc2[C@H](C)OC)c2c3cc(ccc31)-c1csc(n1)C[C@H](NC(=O)[C@@H]1C[C@H]1CF)C(=O)N1CCC[C@H](N1)C(=O)OCC(C)(C)C2] |
| A184 | 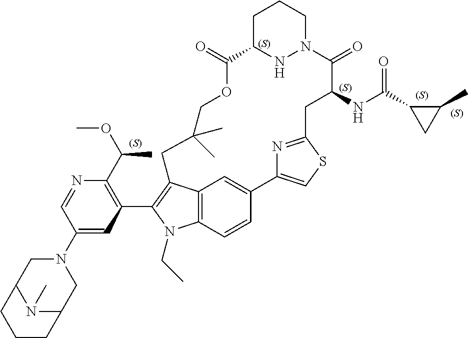 |
| A185 | 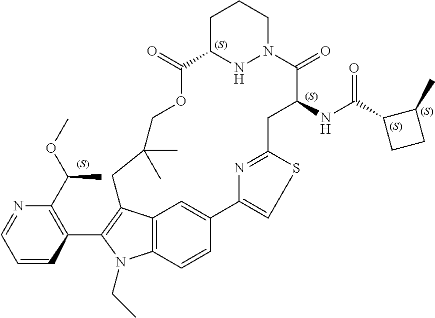 |
| A186 | 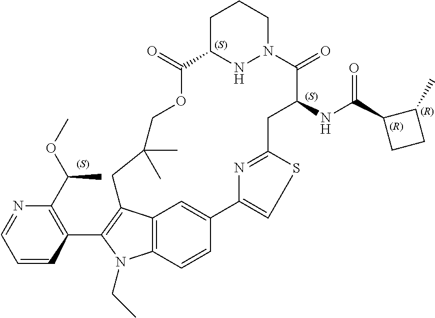 |
| A187 | 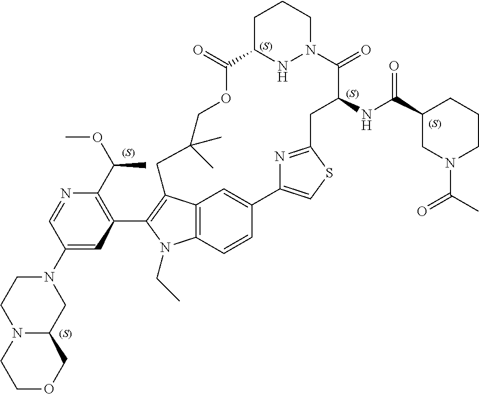 |
| A188 | 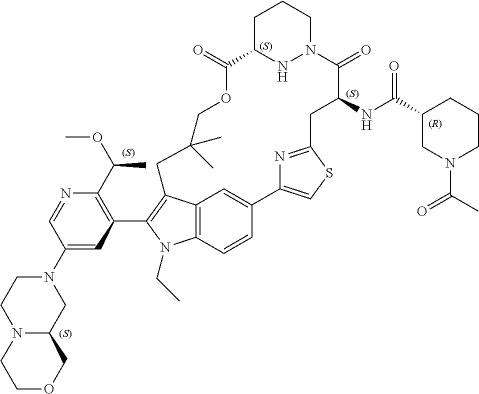 |
| A189 | 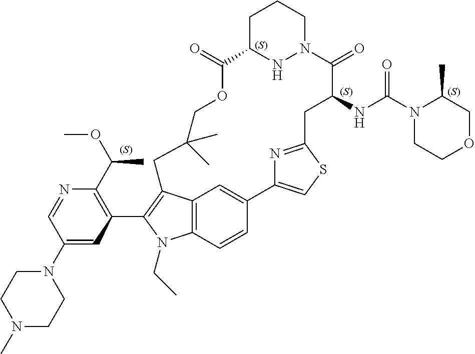 |
| A190 | 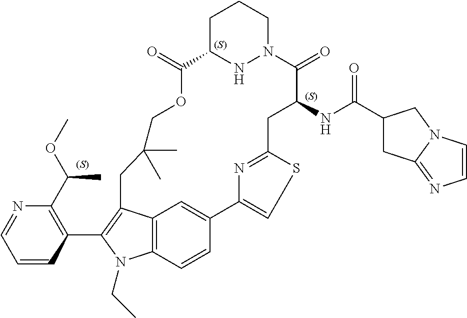 |
| A191 | 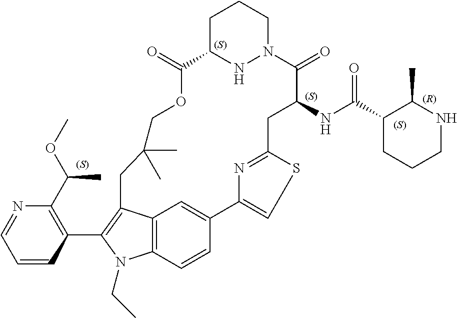 |
| A192 | 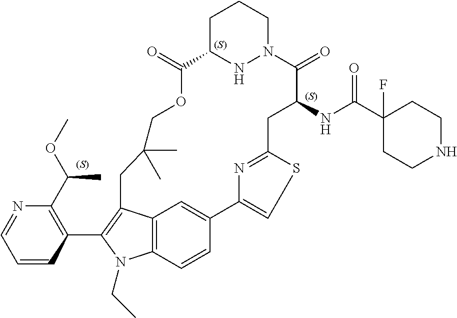 |
| A193 | 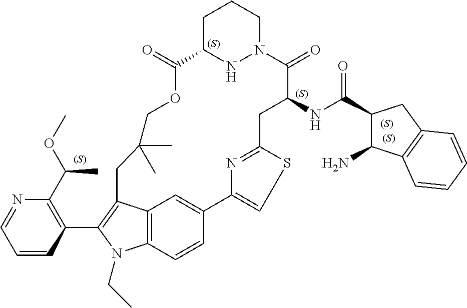 |
| A194 | 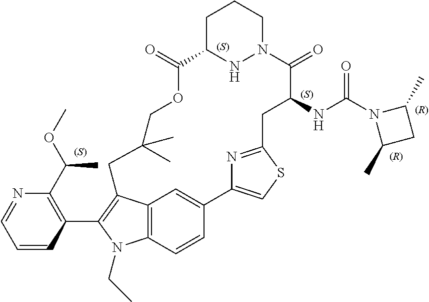 |
| A195 | 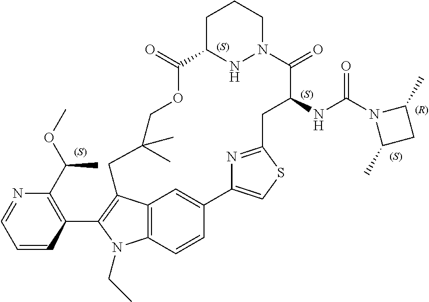 |
| A196 | 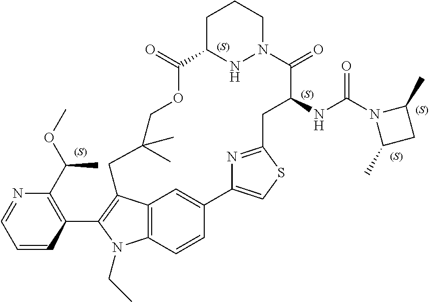 |
| A197 | 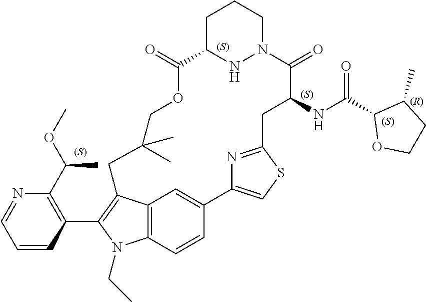 |
| A198 | 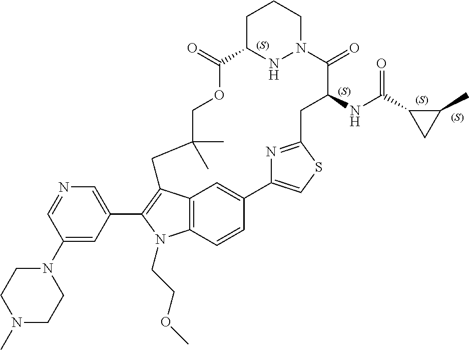 |
| A199 | 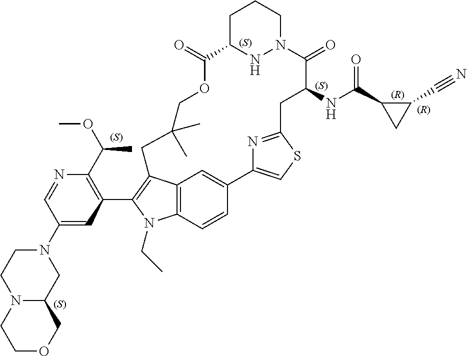 |
| A200 | 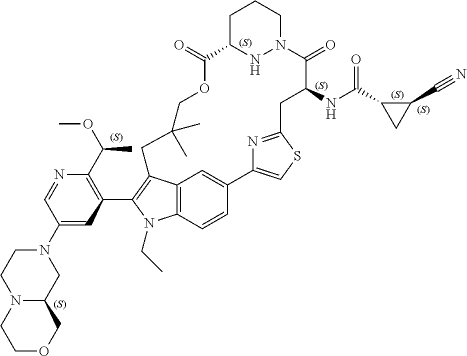 |
| A201 | 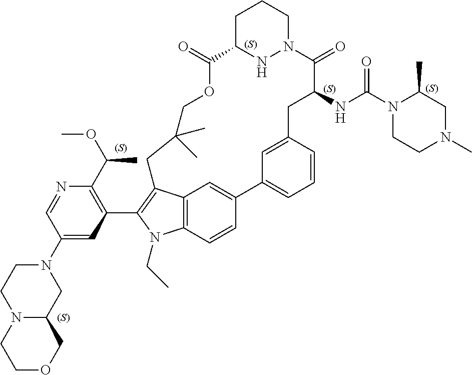 |
| A202 | 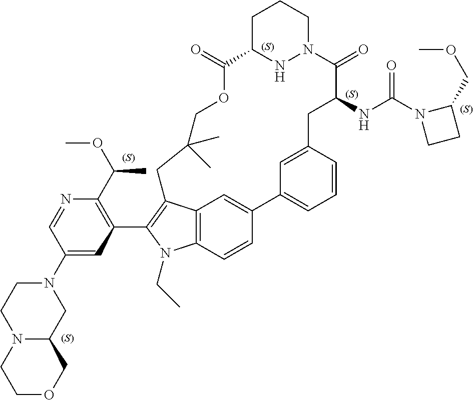 |
| A203 | 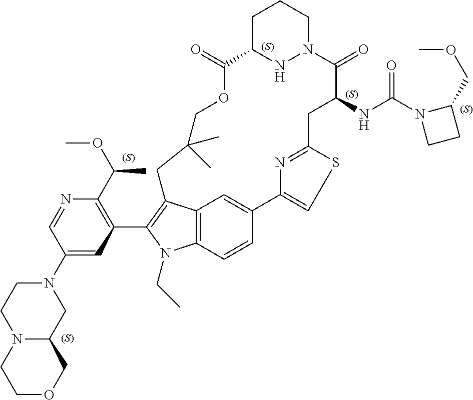 |
| A204 | 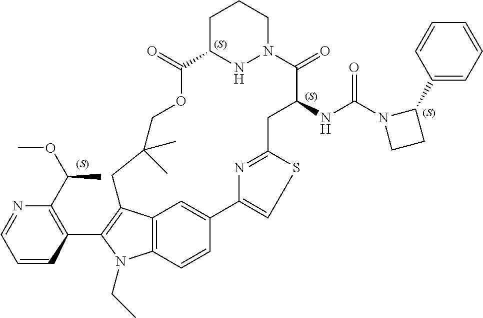 |
| A205 | 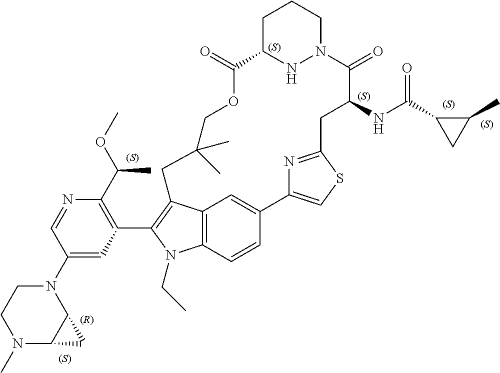 |
| A206 | 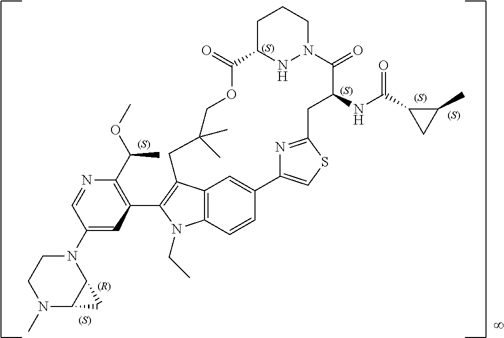 |
| A207 | 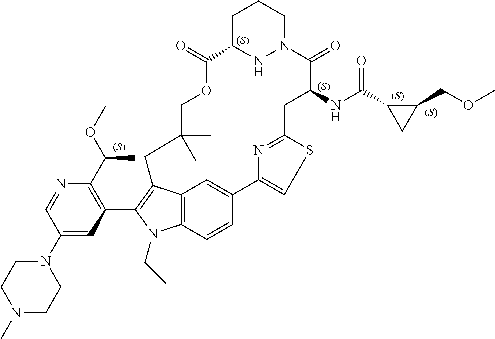 |
| A208 | 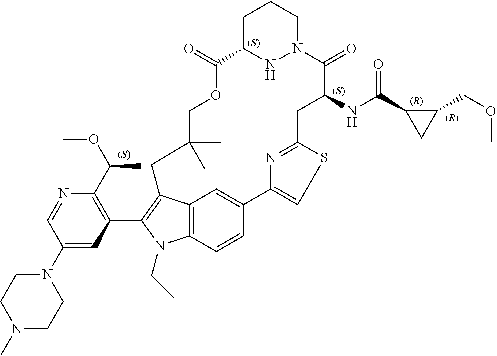 |
| A209 | 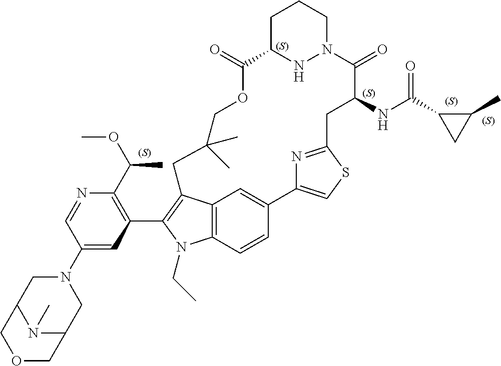 |
| A210 | 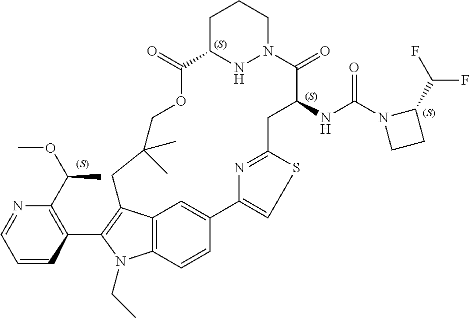 |
| A211 | 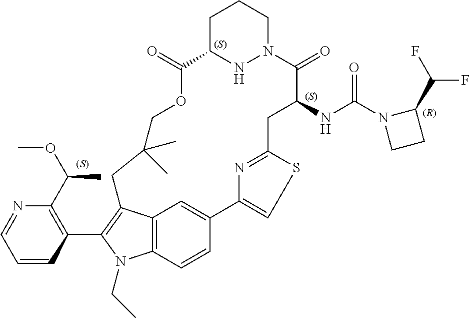 |
| A212 | 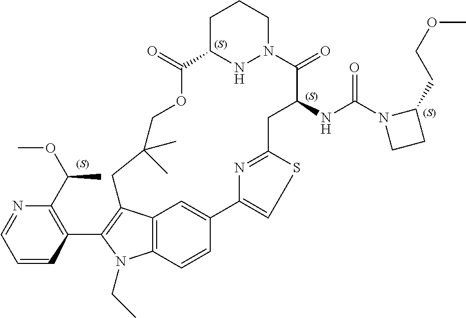 |
| A213 | 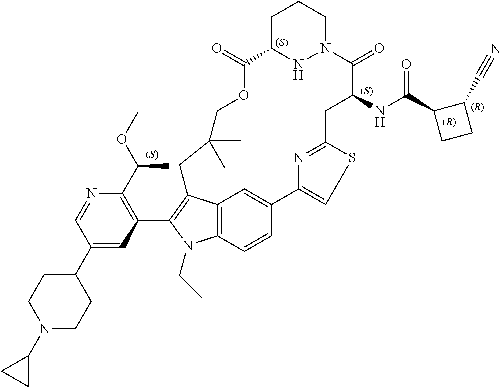 |
| A214 | 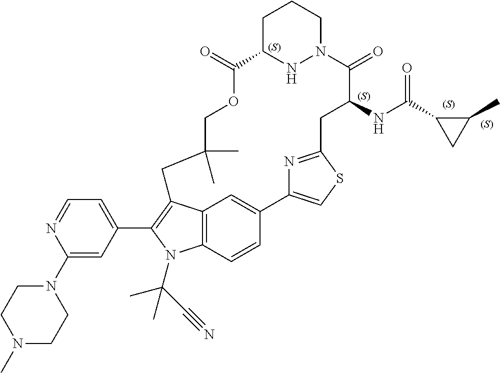 |
| A215 | 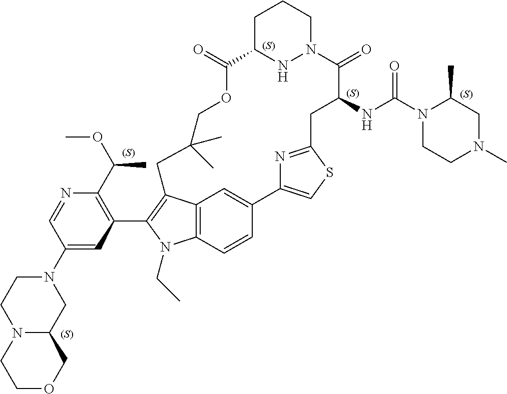 |
| A216 | 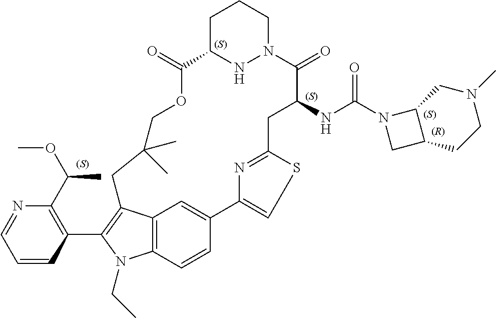 |
| A217 | 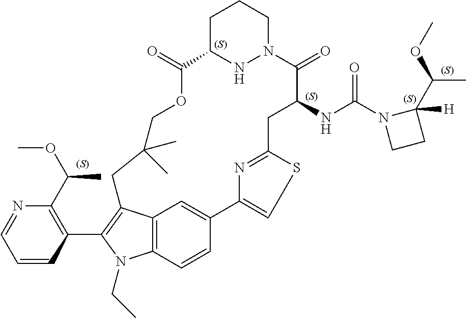 |
| A218 | 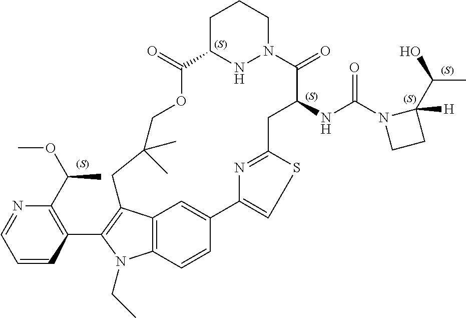 |
| A219 | 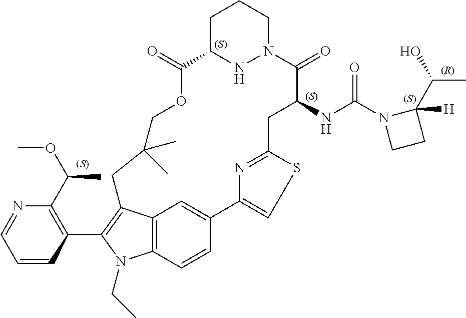 |
| A220 | 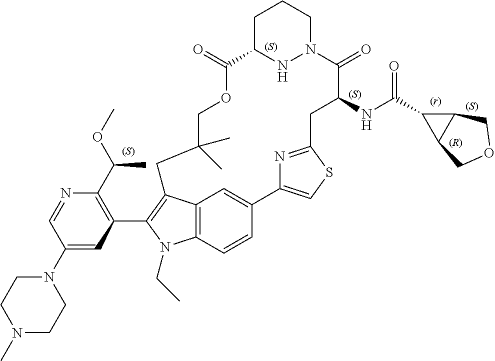 |
| A221 | 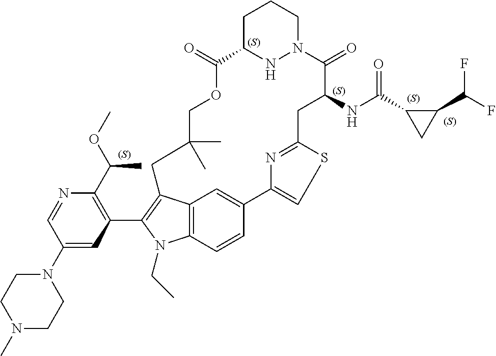 |
| A222 | 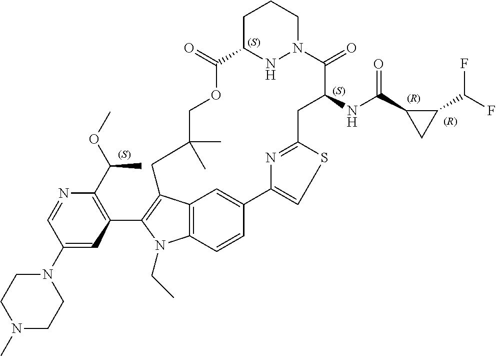 |
| A223 | 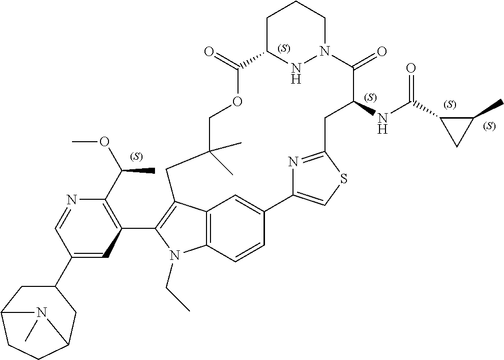 |
| A224 | 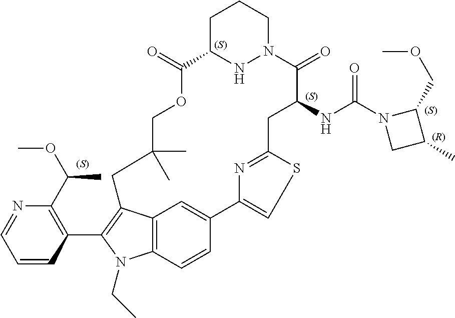 |
| A225 | 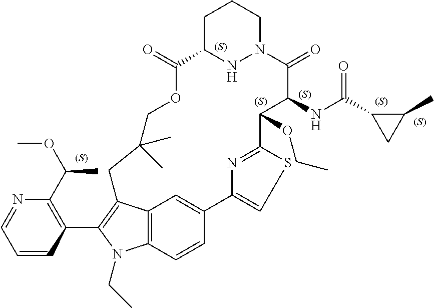 |
| A226 | 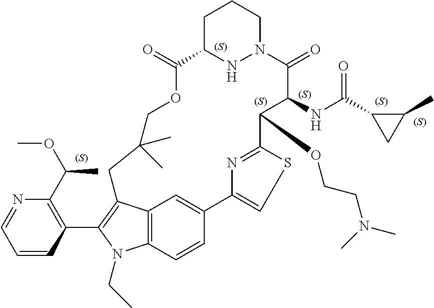 |
| A227 | 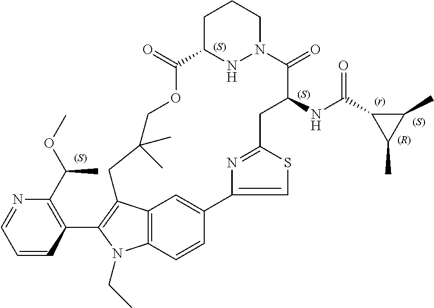 |
| A228 | 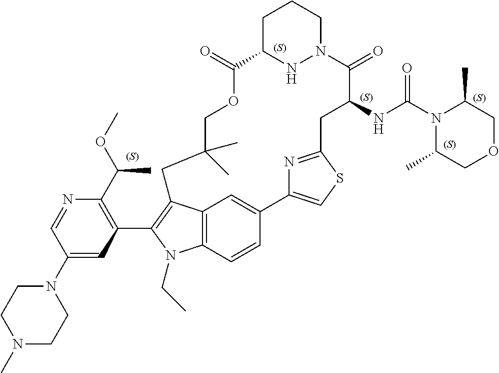 |
| A229 | 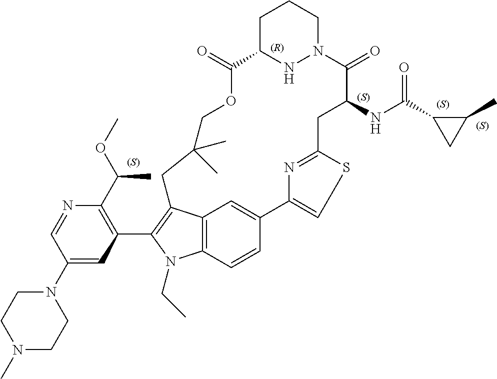 |
| A230 | 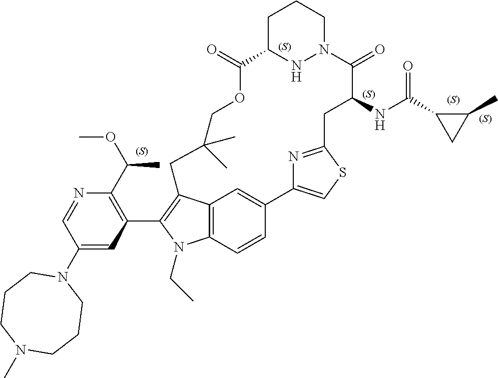 |
| A231 | 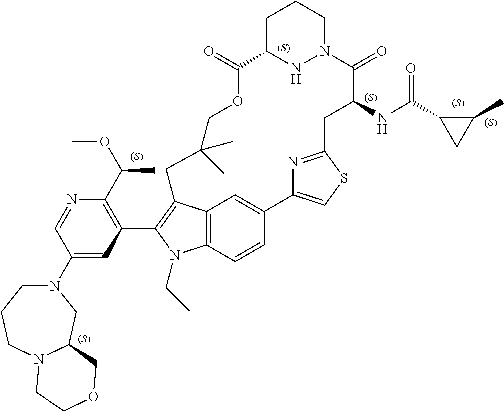 |
| A232 | 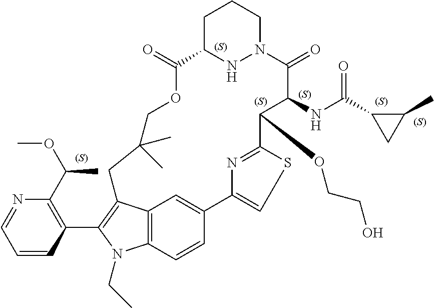 |
| A233 | 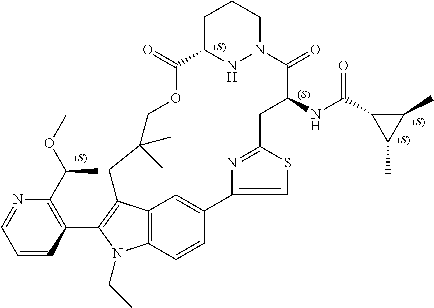 |
| A234 | 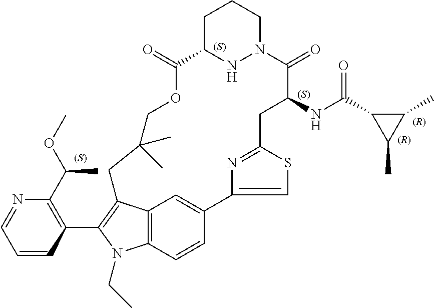 |
| A235 | 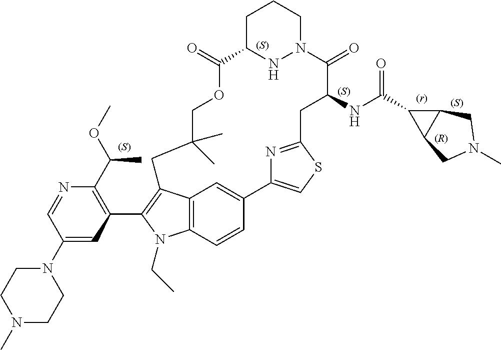 |
| A236 | 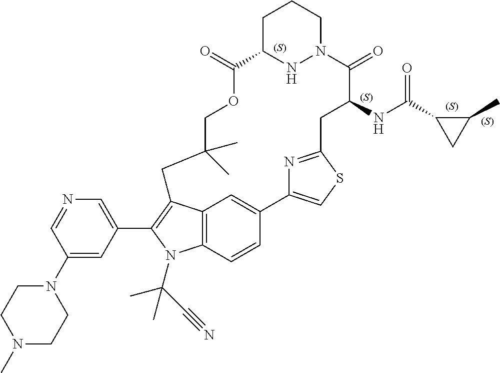 |
| A237 | 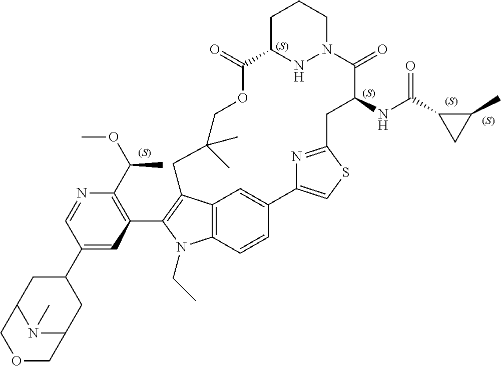 |
| A238 | 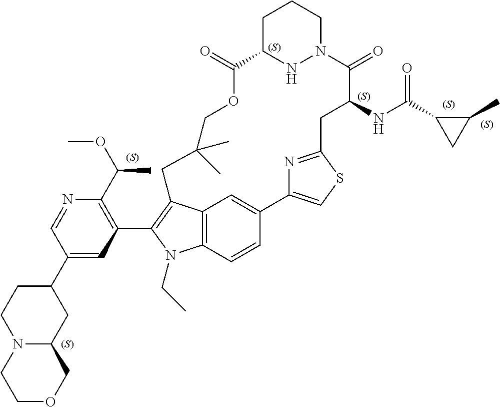 |
| A239 | 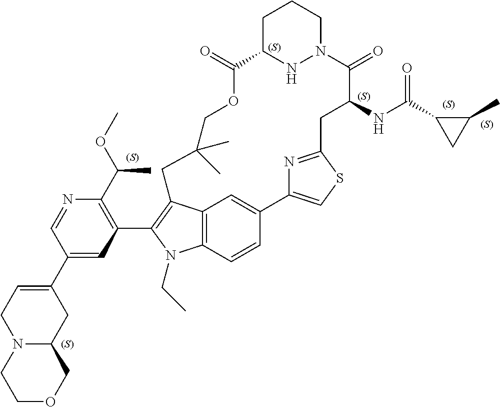 |
| A240 | 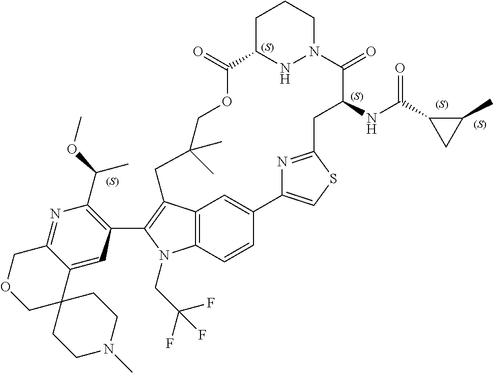 |
| A241 | 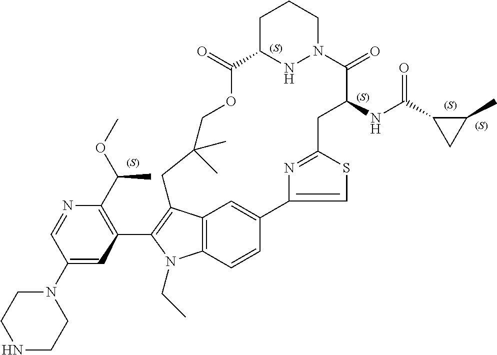 |
| A242 | 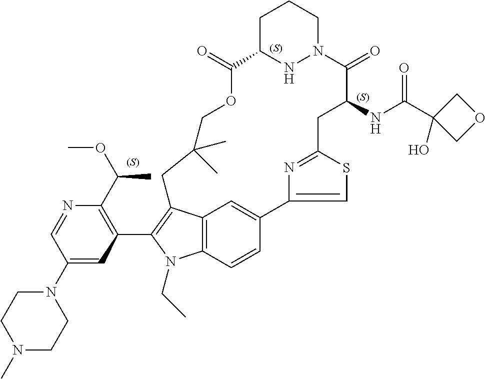 |
| A243 | 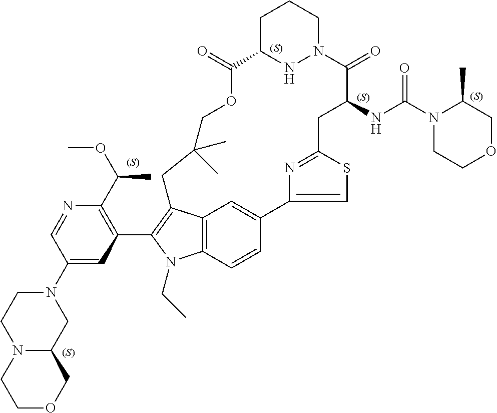 |
| A244 | 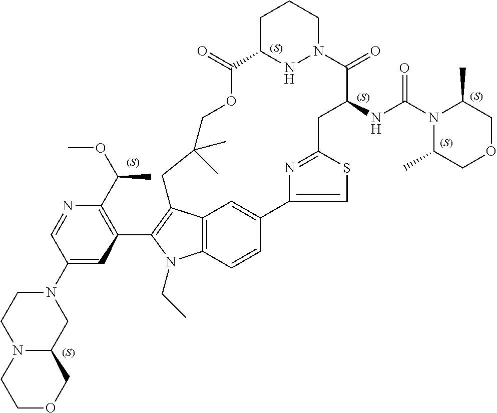 |
| A245 | 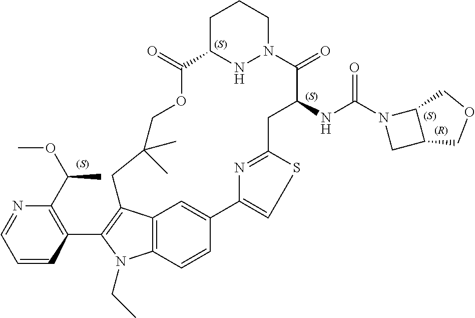 |
| A246 | 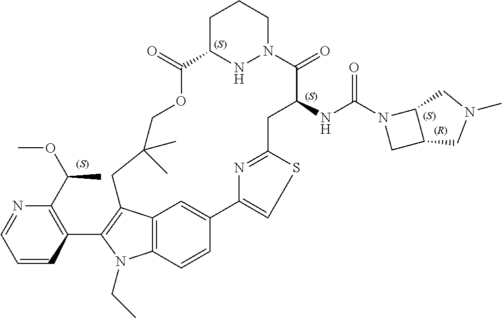 |
| A247 | 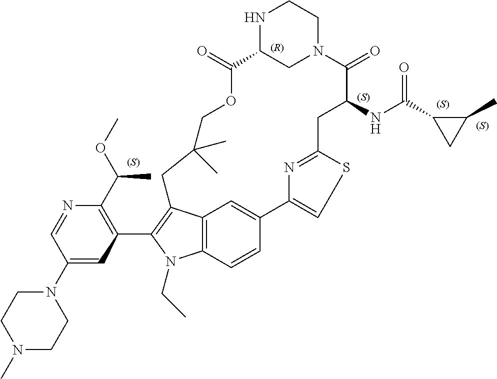 |
| A248 | 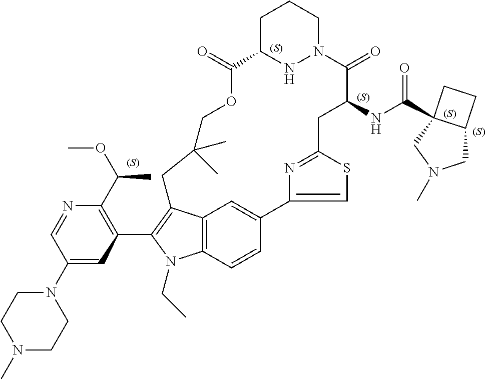 |
| A249 | 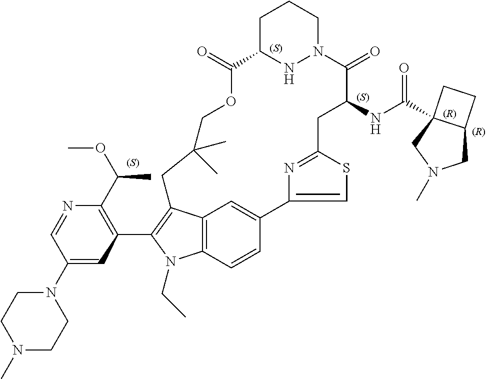 |
| A250 | 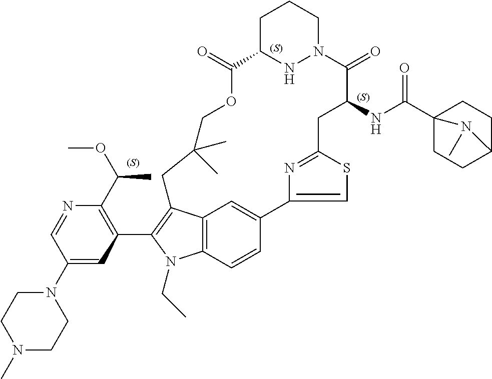 |
| A251 | 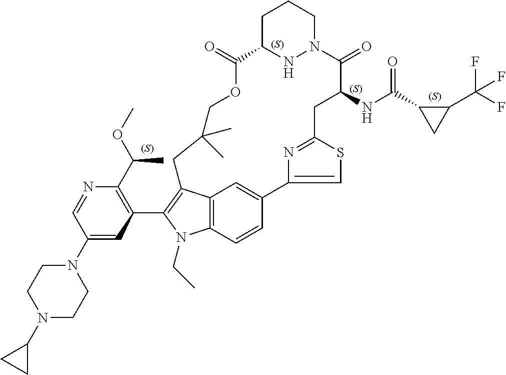 |
| A252 | 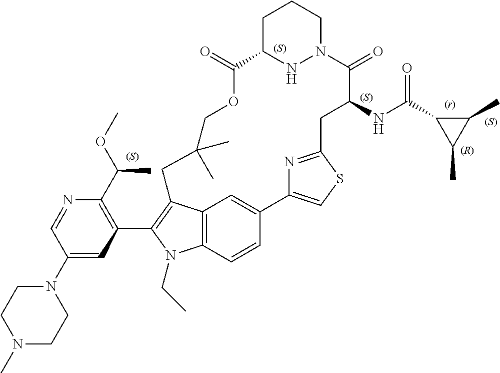 |
| A253 | 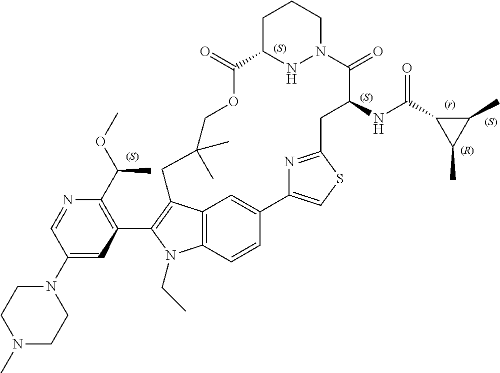 |
| A254 | 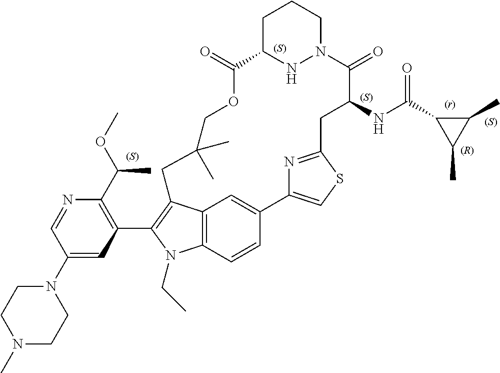 |
| A255 | 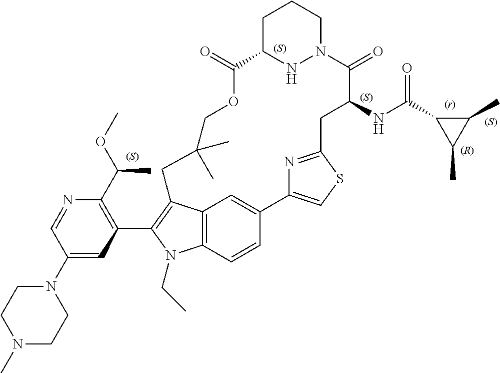 |
| A256 | 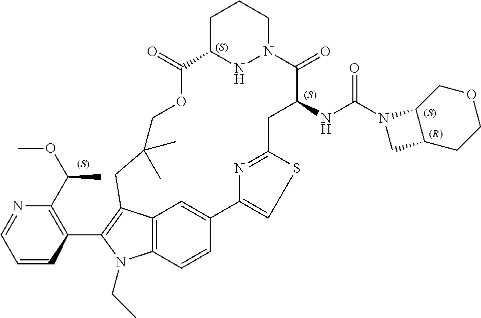 |
| A257 | 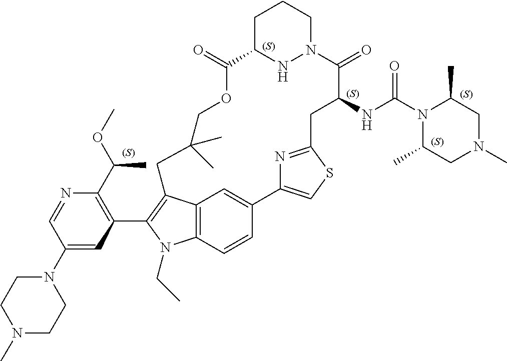 |
| A258 | 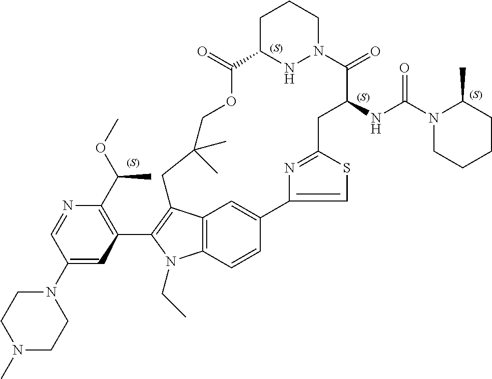 |
| A259 | 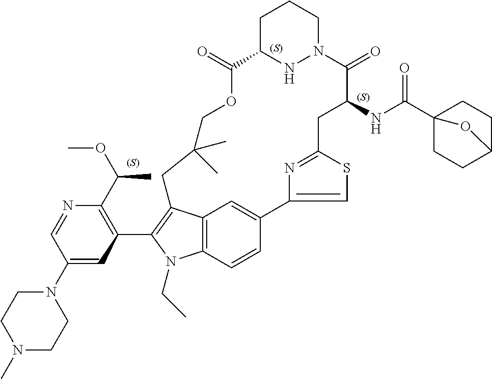 |
| A260 | 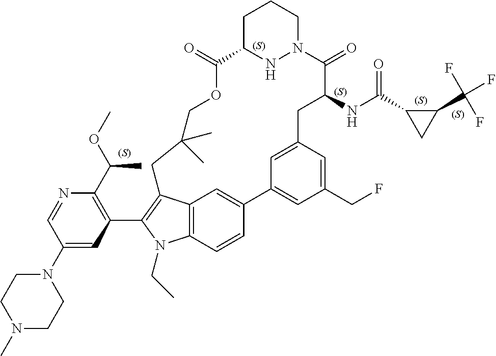 |
| A261 | 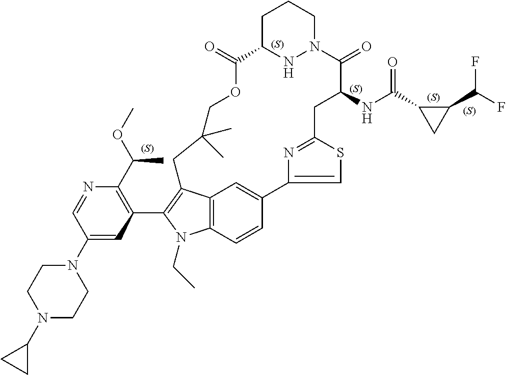 |
| A262 | 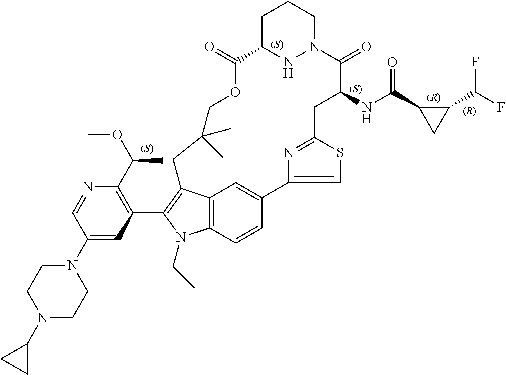 |
| A263 | 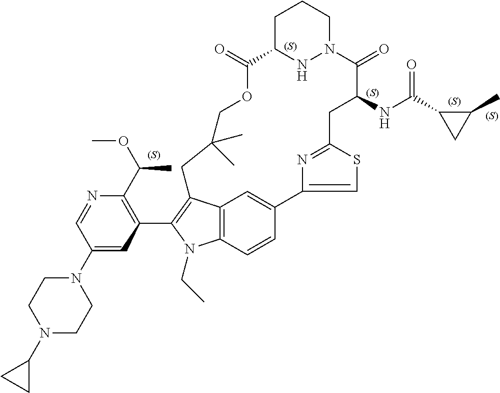 |
| A264 | 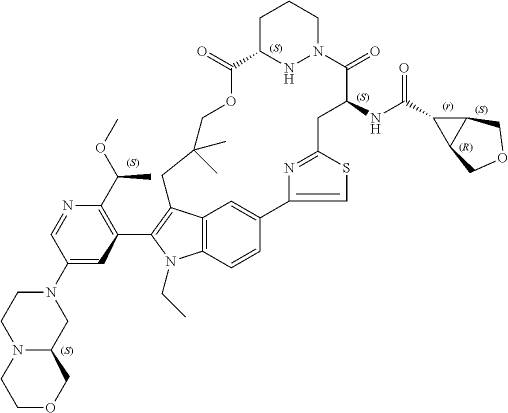 |
| A265 | 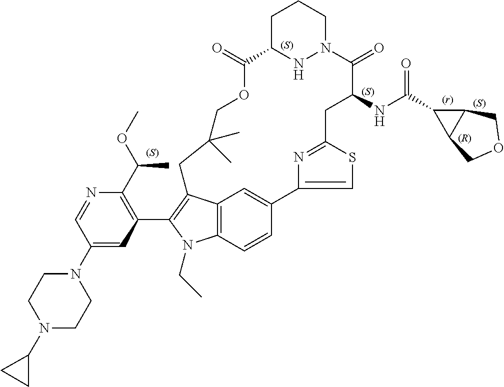 |
| A266 | 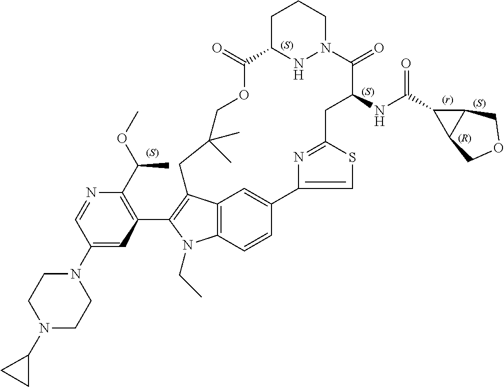 |
| A267 | 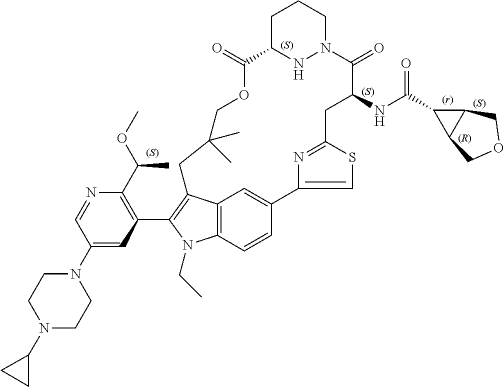 |
| A268 | 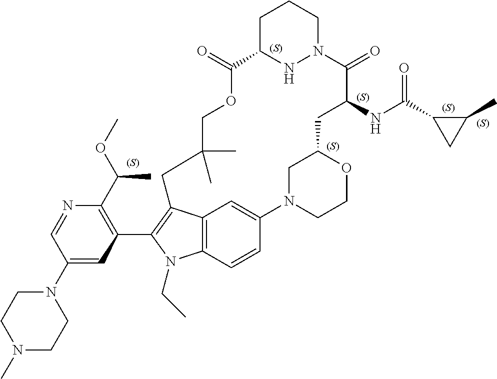 |
| A269 | 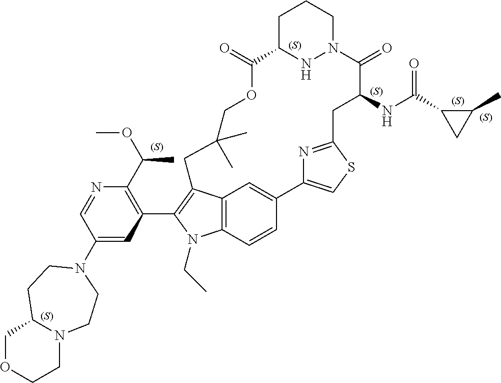 |
| A270 | 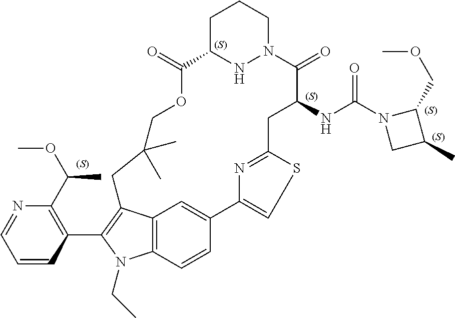 |
| A271 | 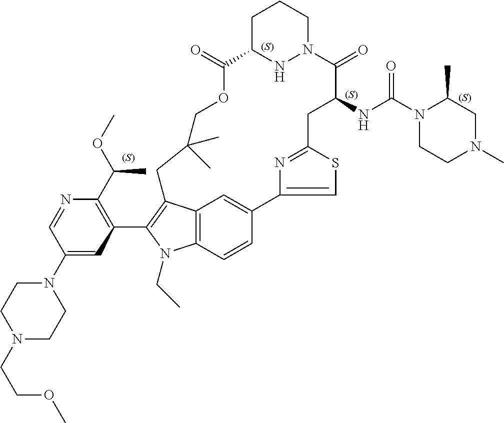 |
| A272 | 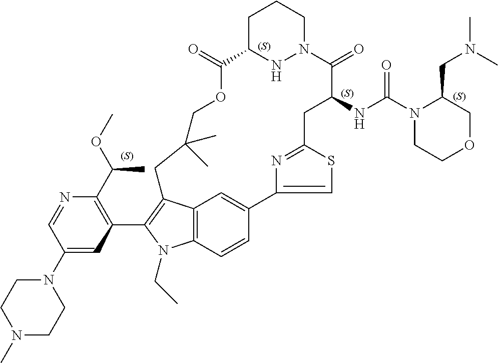 |
| A273 | 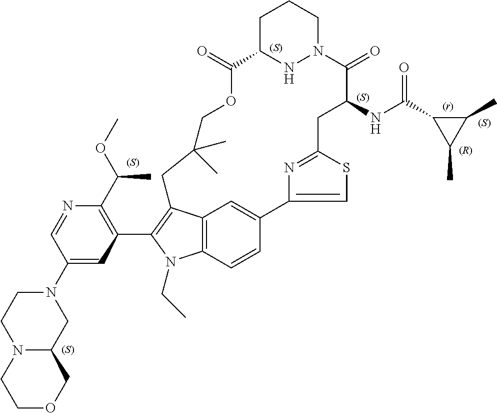 |
| A274 | 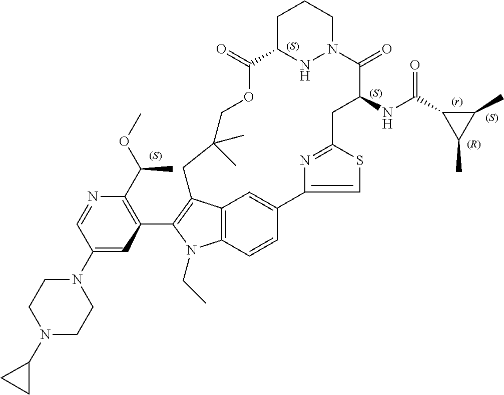 |
| A275 | 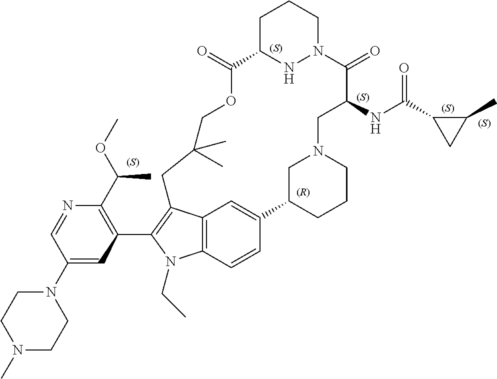 |
| A276 | 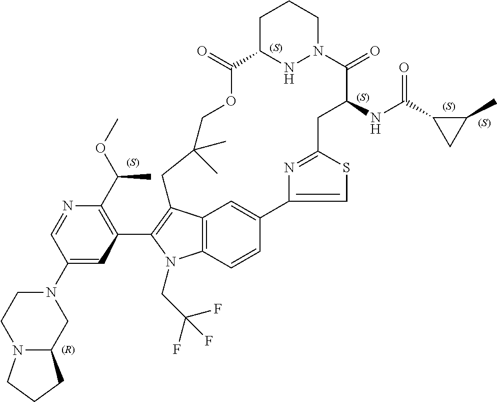 |
| A277 | 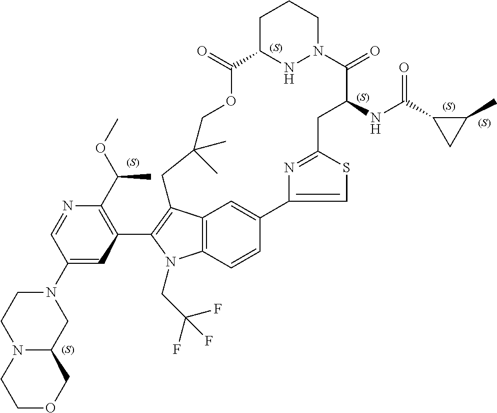 |
| A278 | 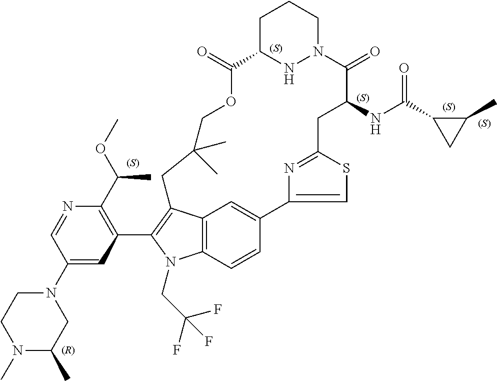 |
| A279 | 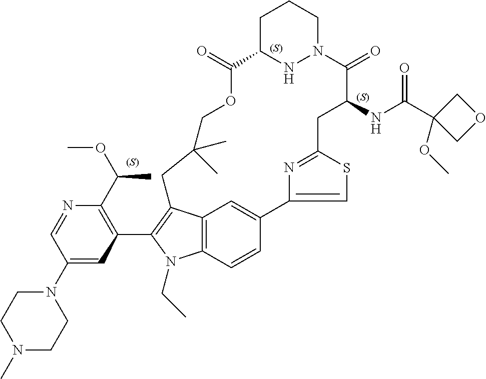 |
| A280 | 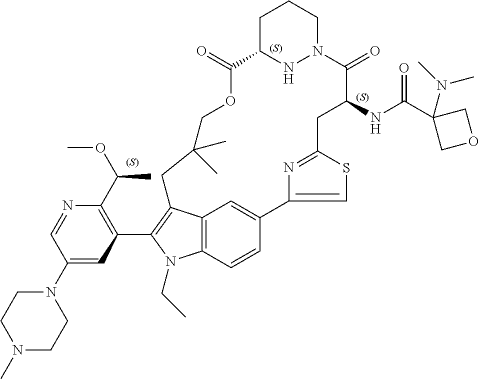 |
| A281 | 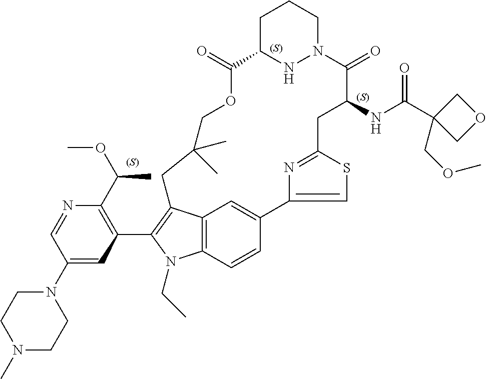 |
| A282 | 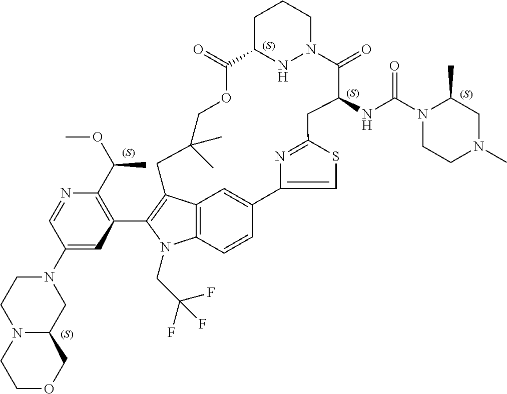 |
| A283 | 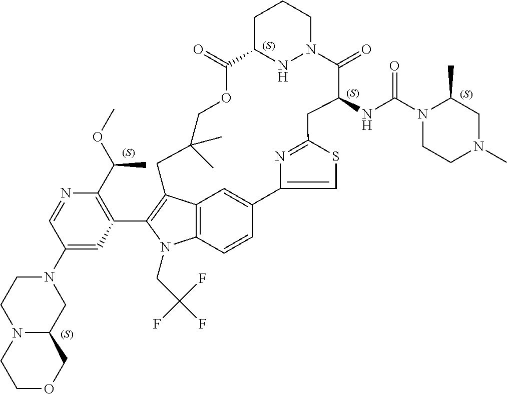 |
| A284 | 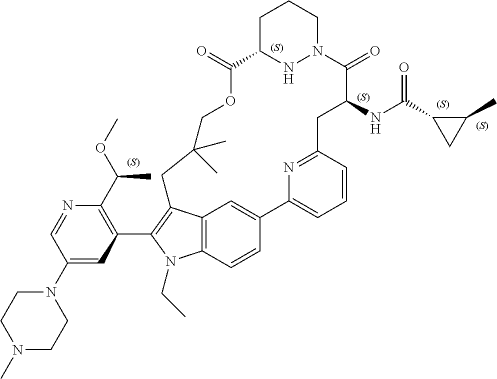 |
| A285 | 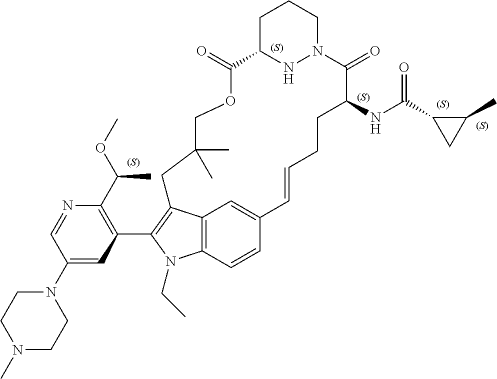 |
| A286 | 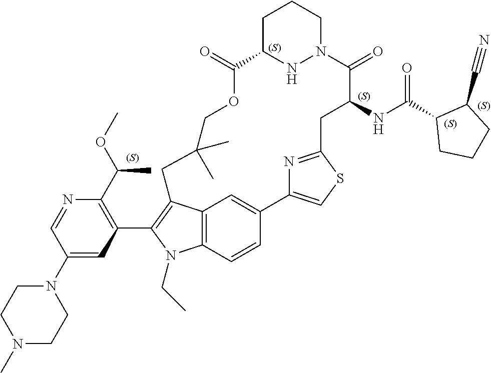 |
| A287 | 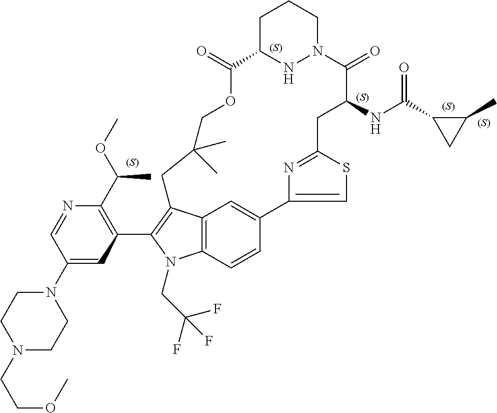 |
| A288 | 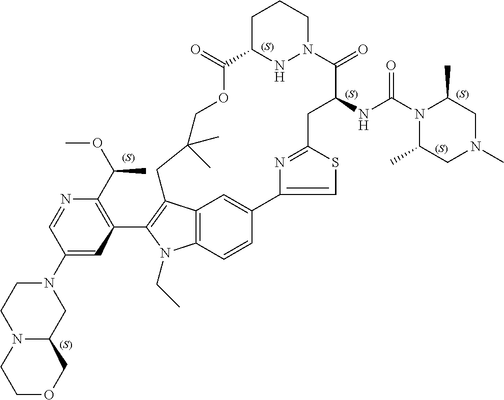 |
| A289 | 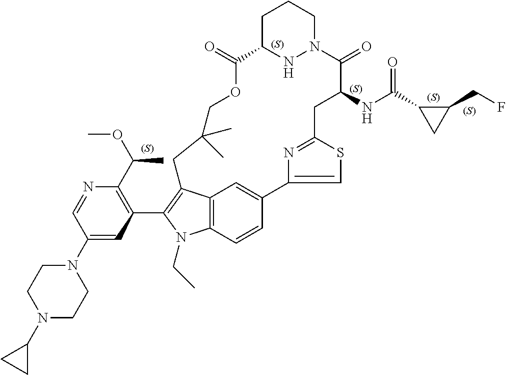 |
| A290 | 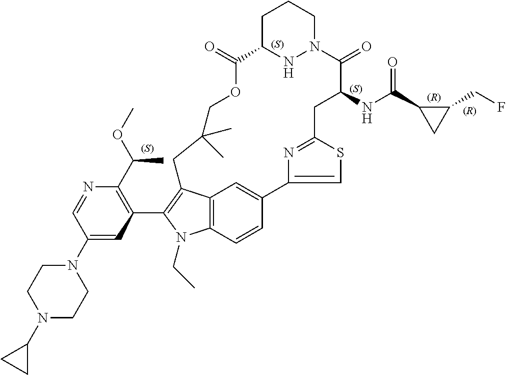 |
| A291 | 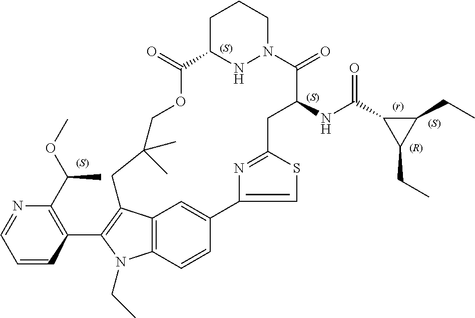 |
| A292 | 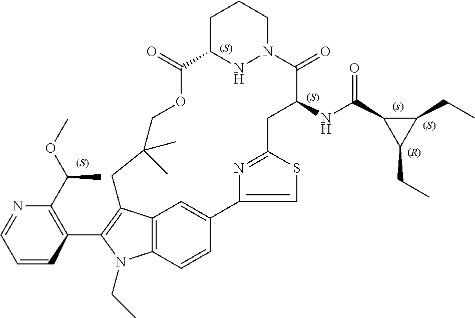 |
| A293 | 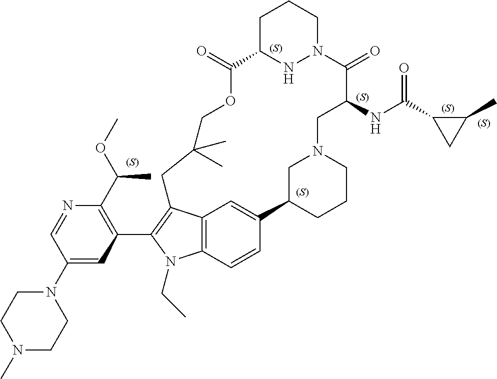 |
| A294 | 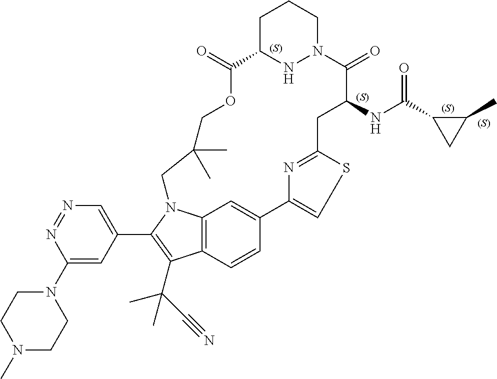 |
| A295 | 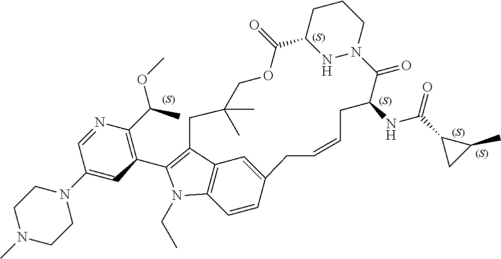 |
| A296 | 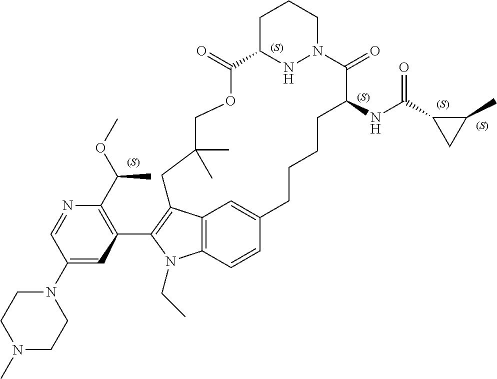 |
| A297 | 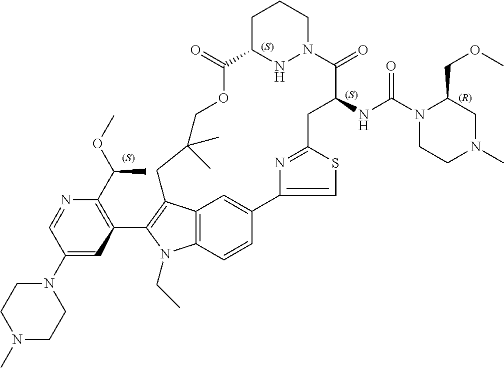 |
| A298 | 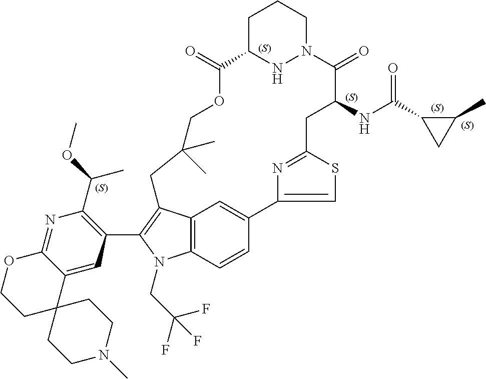 |
| A299 | 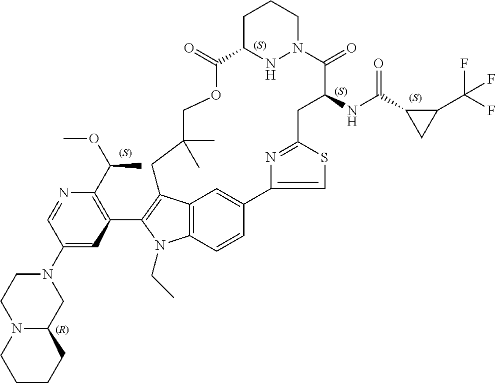 |
| A300 | 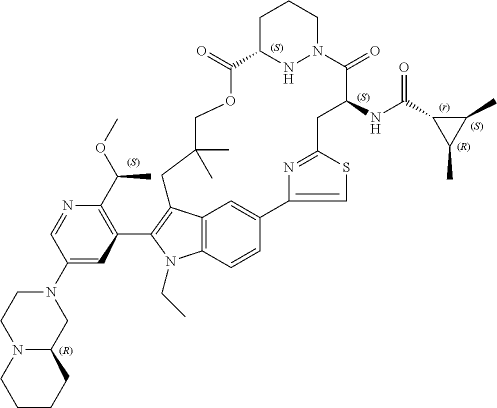 |
| A301 | 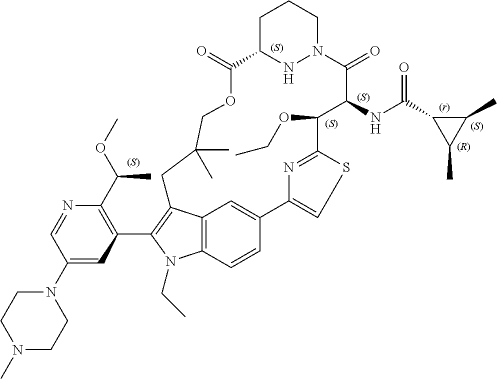 |
| A302 | 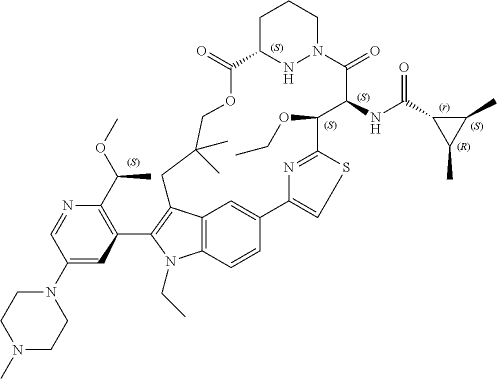 |
| A303 | 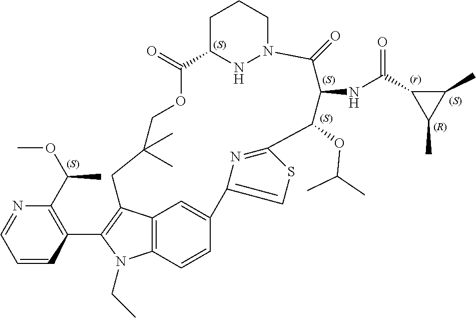 |
| A304 | 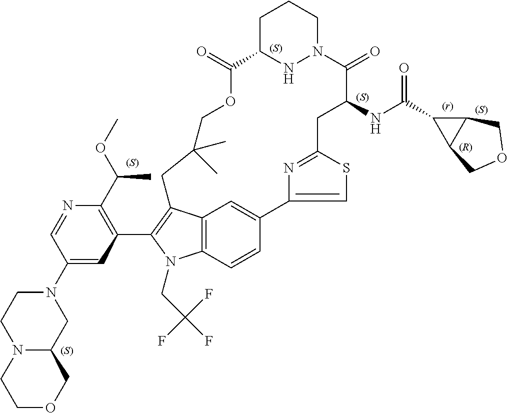 |
| A305 | 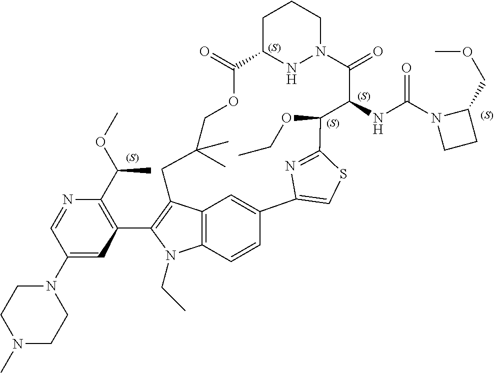 |
| A306 | 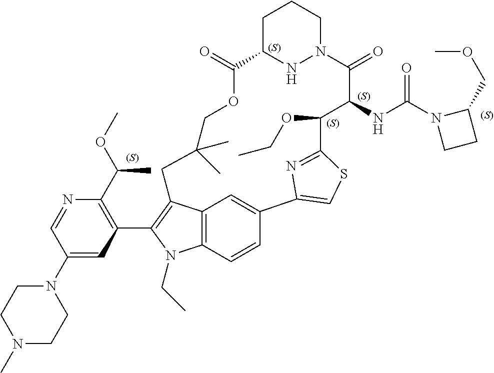 |
| A307 | 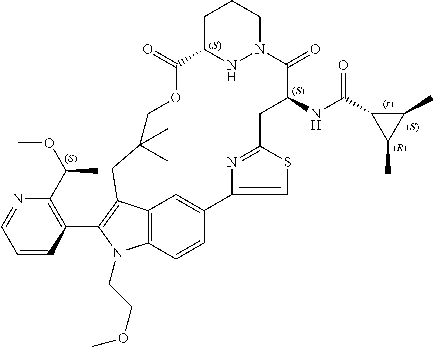 |
| A308 | 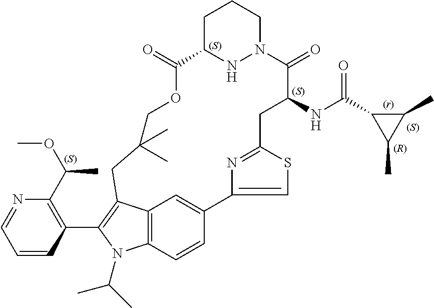 |
| A309 | 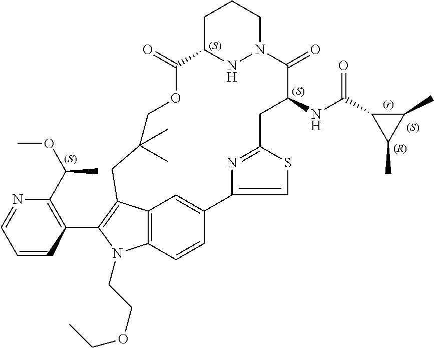 |
| A310 | 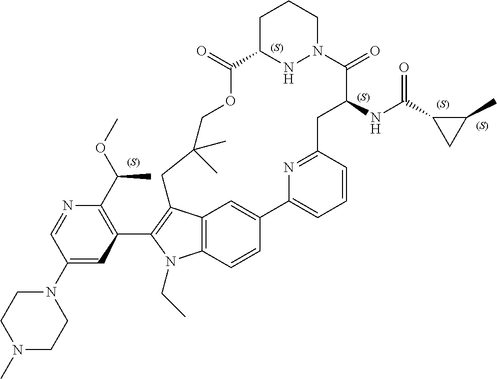 |
| A311 | 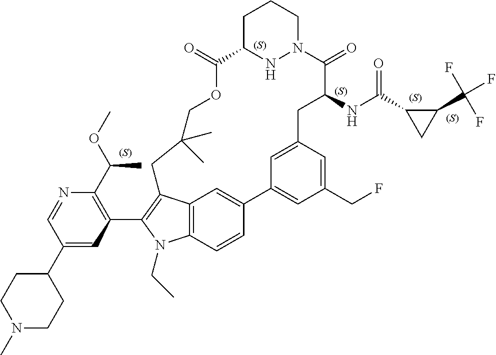 |
| A312 | 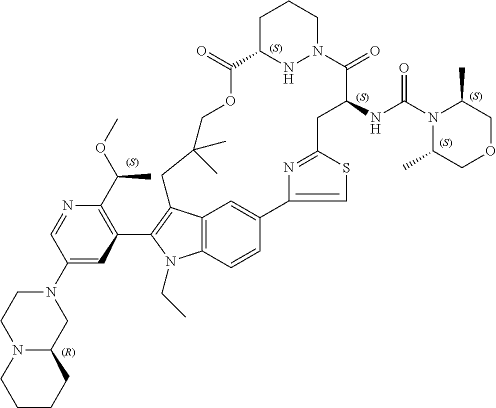 |
| A313 | 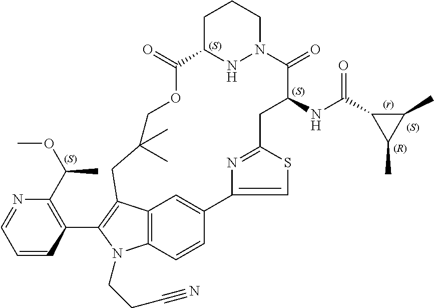 |
| A314 | 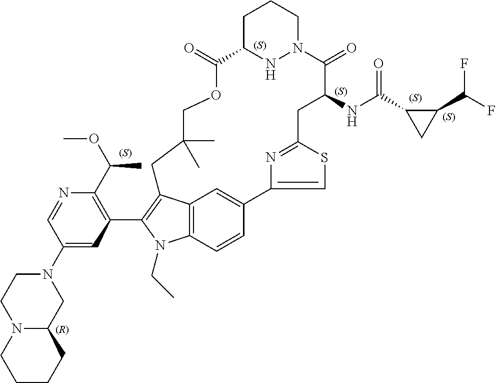 |
| A315 | 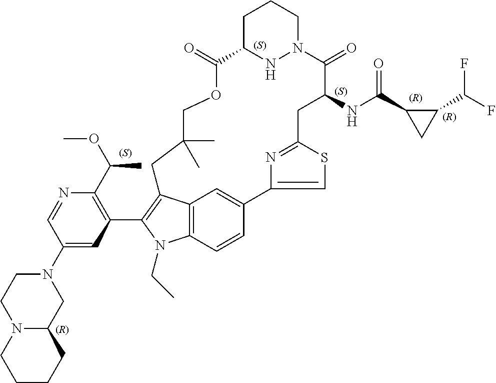 |
| A316 | 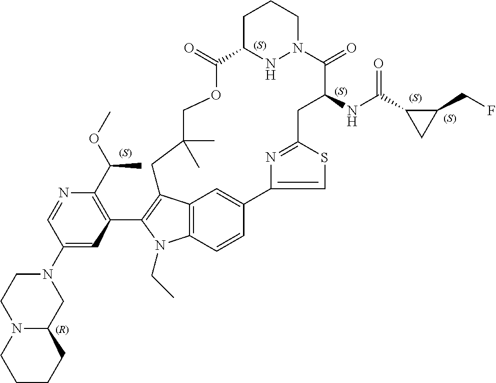 |
| A317 | 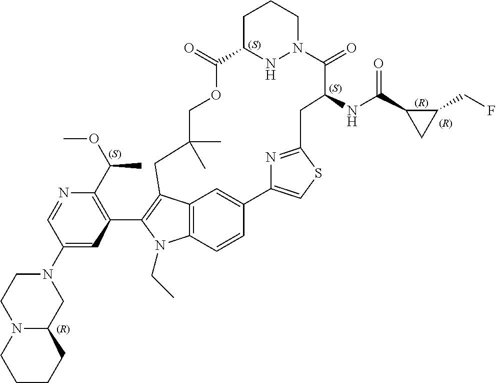 |
| A318 | 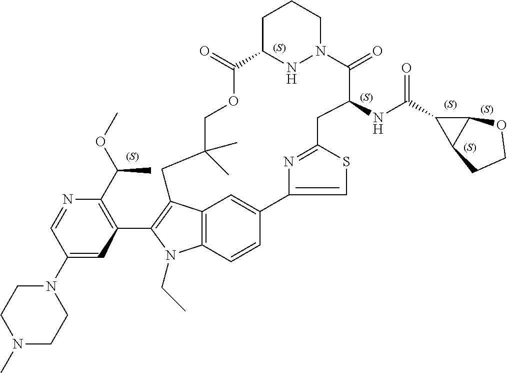 |
| A319 | 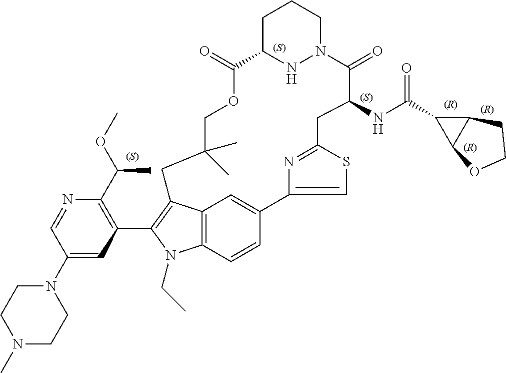 |
| A320 | 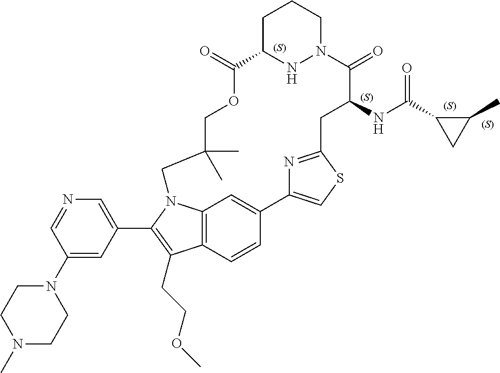 |
| A321 | 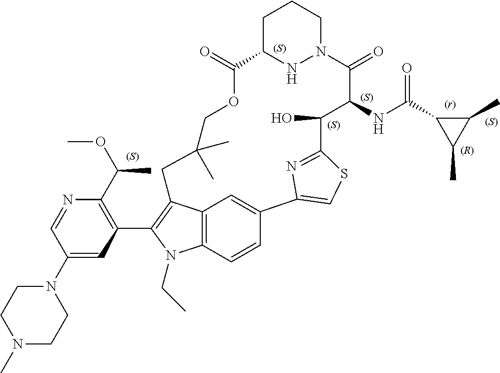 |
| A322 | 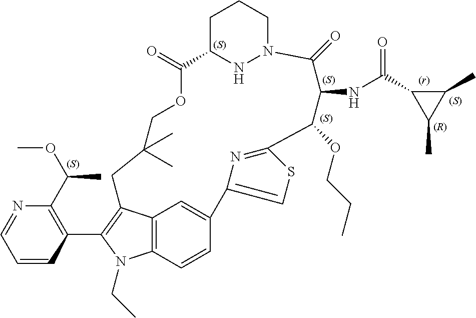 |
| A323 | 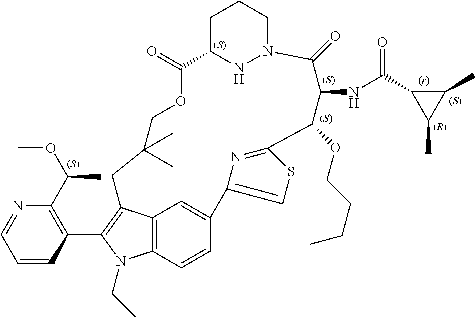 |
| A324 | 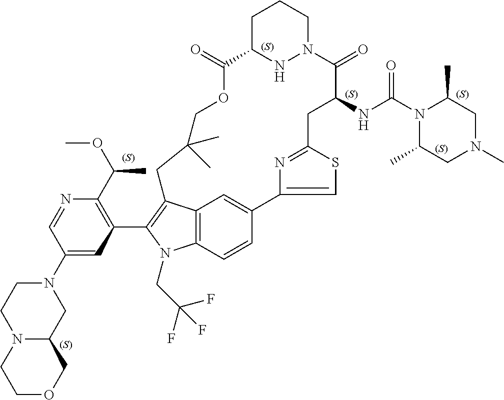 |
| A325 | 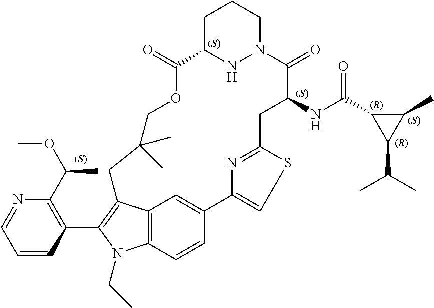 |
| A326 | 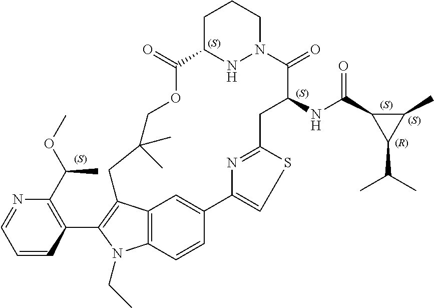 |
| A327 | 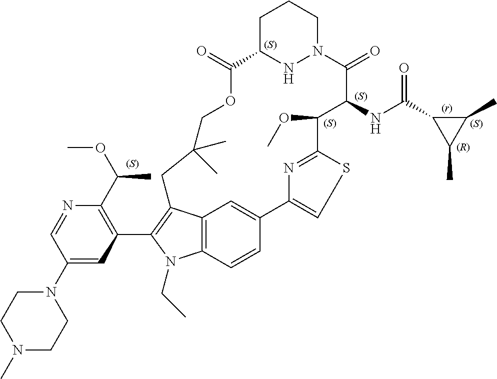 |
| A328 | 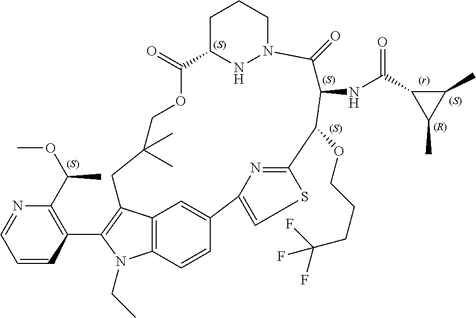 |
| A329 | 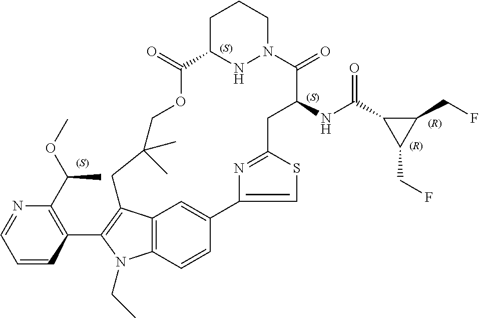 |
| A330 | 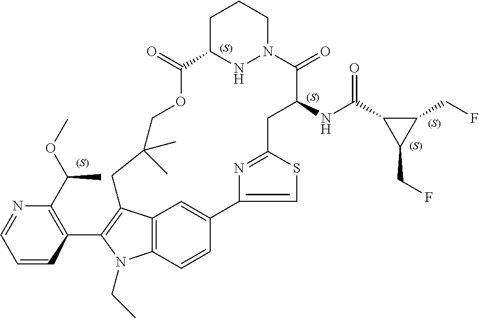 |
| A331 | 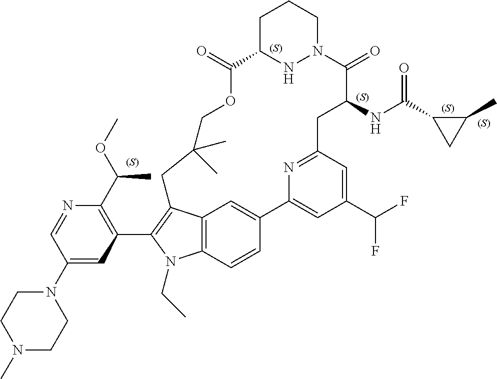 |
| A332 | 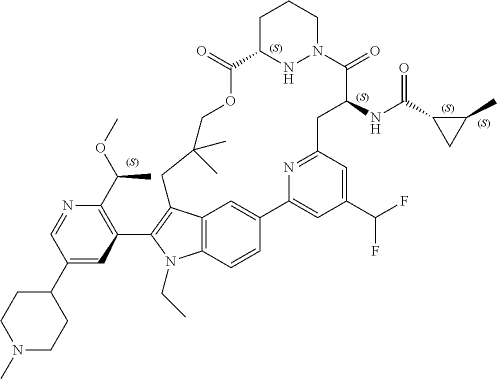 |
| A333 | 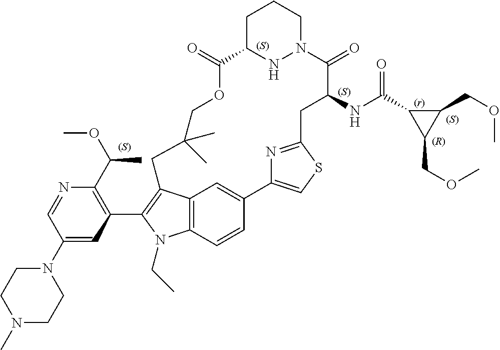 |
| A334 | 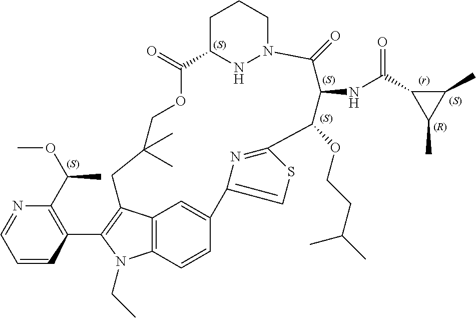 |
| A335 | 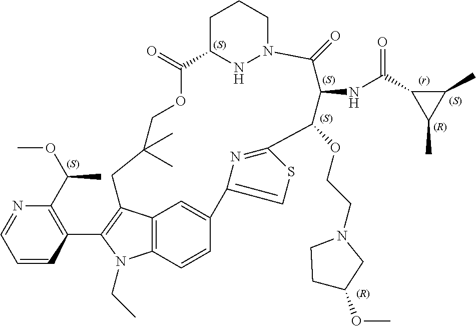 |
| A336 | 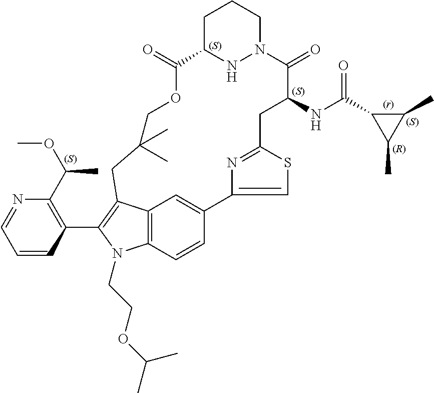 |
| A337 | 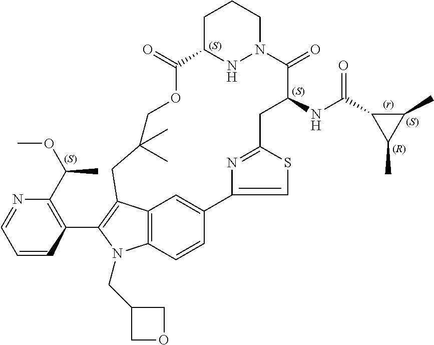 |
| A338 | 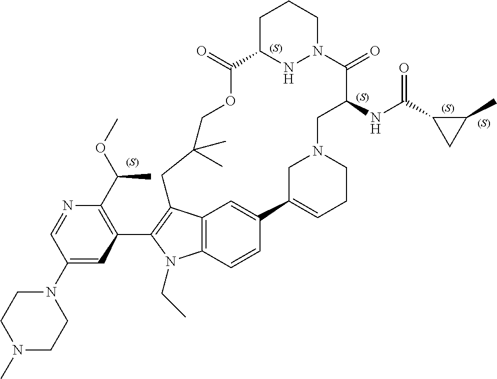 |
| A339 | 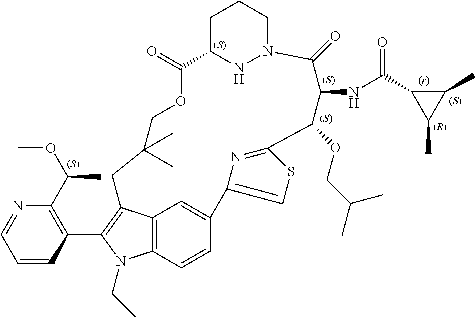 |
| A340 | 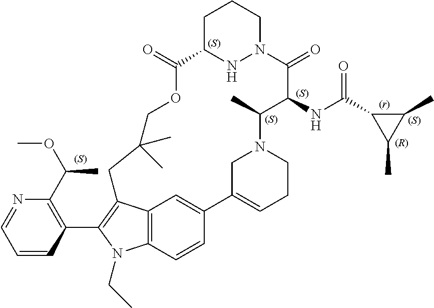 |
| A341 | 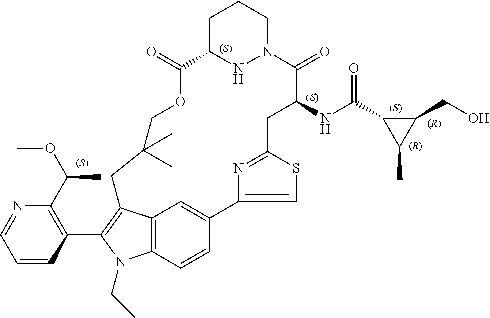 |
| A342 | 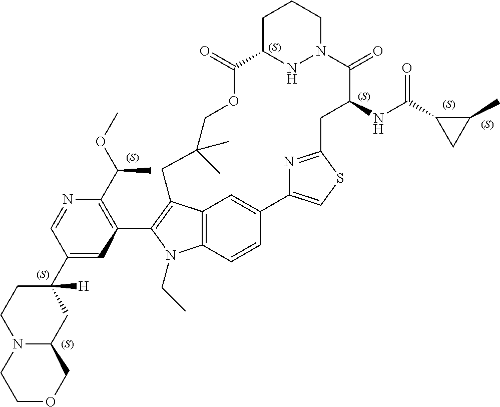 |
| A343 | 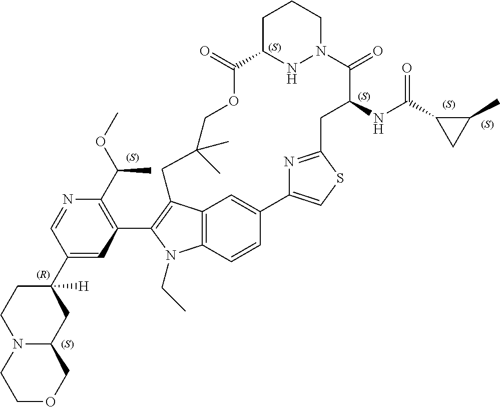 |
| A344 | 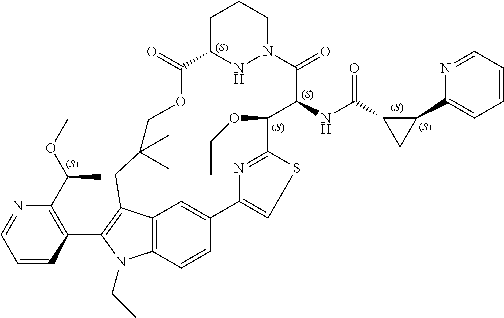 |
| A345 | 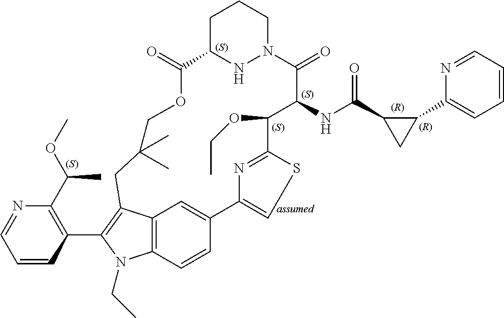 |
| A346 | 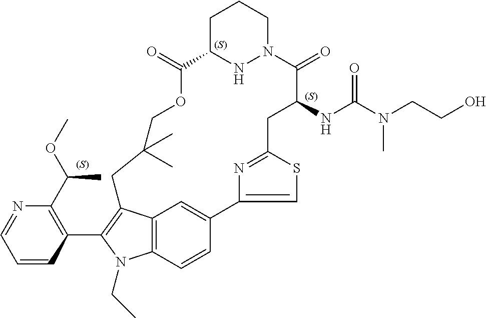 |
| A347 | 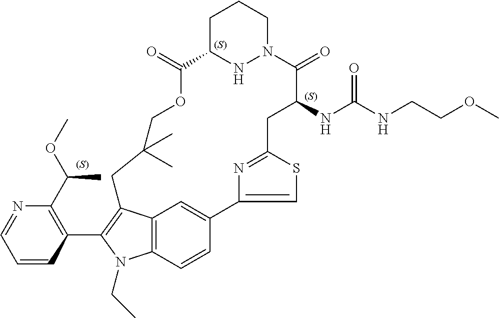 |
| A348 | 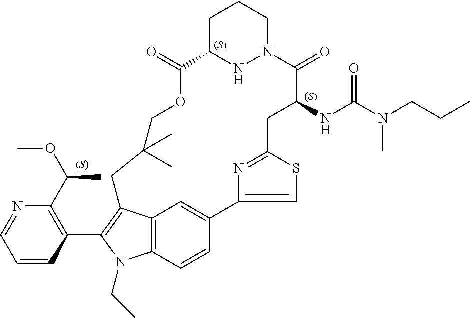 |
| A349 | 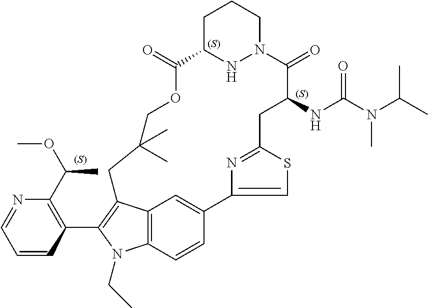 |
| A350 | 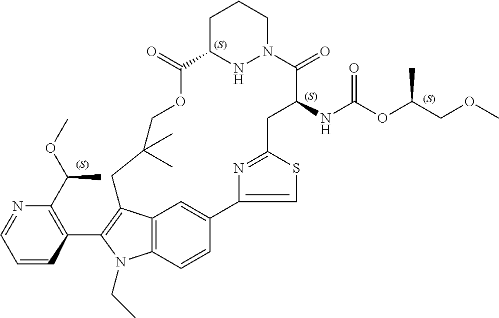 |
| A351 | 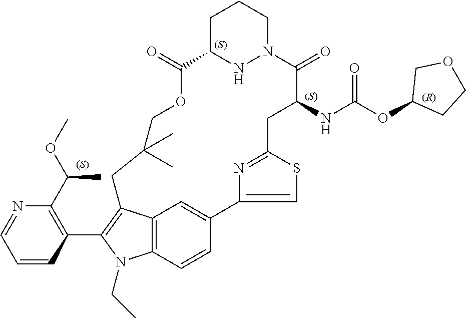 |
| A352 | 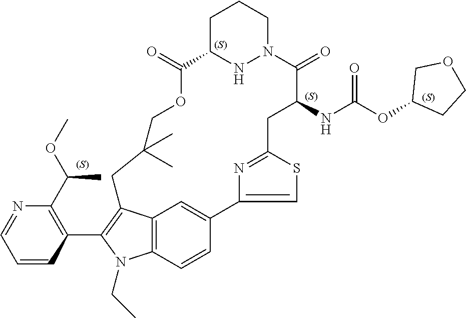 |
| A353 | 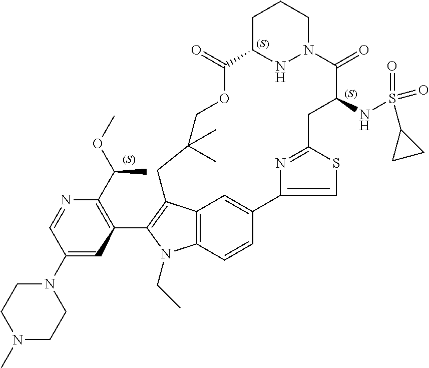 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

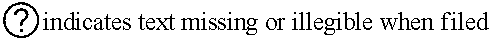

In some embodiments, a compound of the present invention is selected from Table 1-1, or a pharmaceutically acceptable salt or stereoisomer thereof. In some embodiments, a compound of the present invention is selected from Table 1-1, or a pharmaceutically acceptable salt or atropisomer thereof.

TABLE 1-1
Certain Compounds of the Present Invention

| Ex # | Structure |
|---|---|
| A354 | 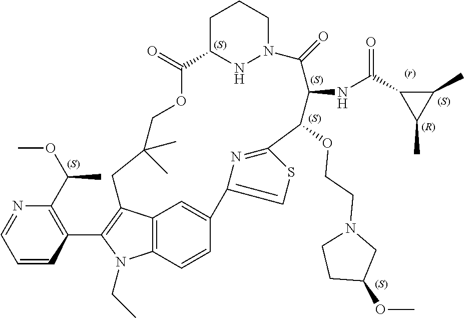 |
| A355 | 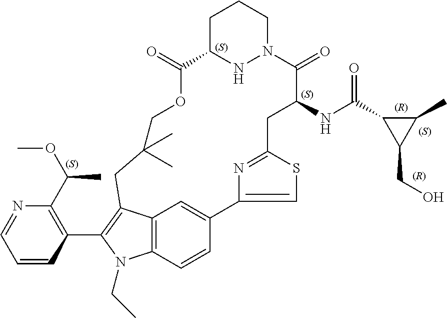 |
| A356 | 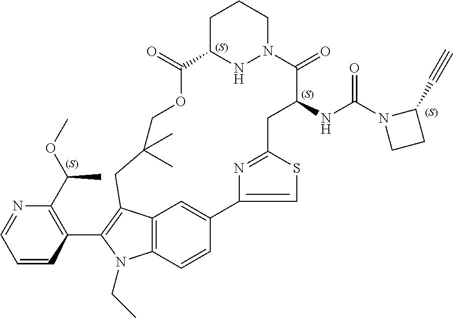 |
| A357 | 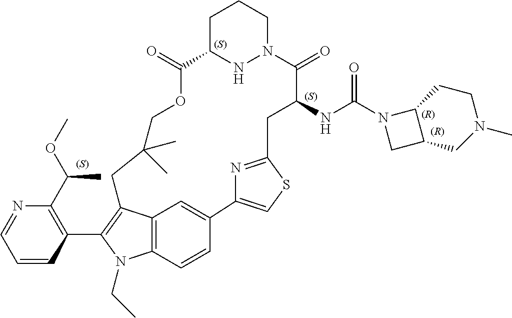 |
| A358 | 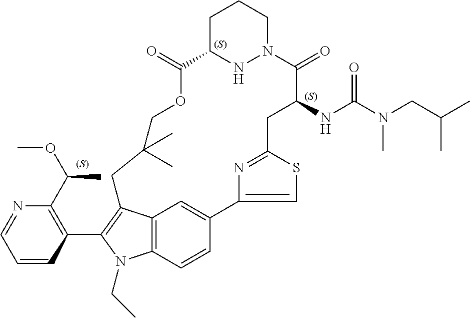 |
| A359 | 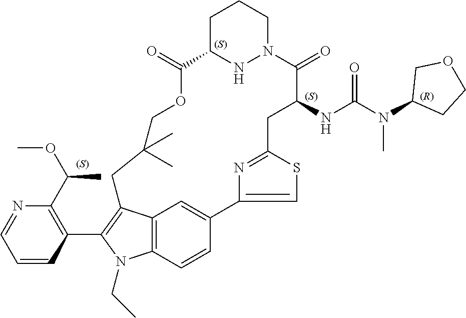 |
| A360 | 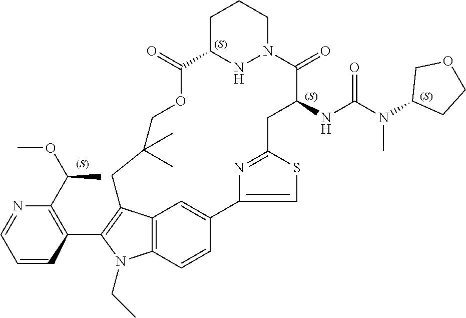 |
| A361 | 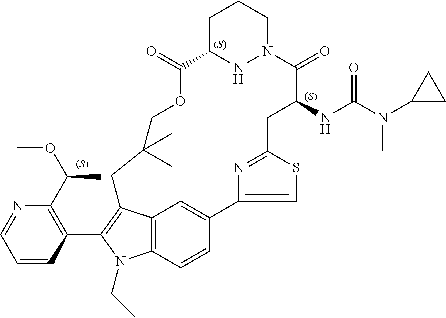 |
| A362 | 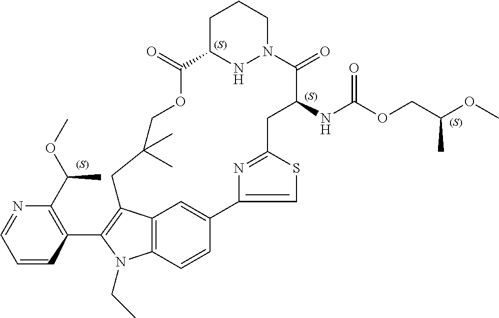 |
| A363 | 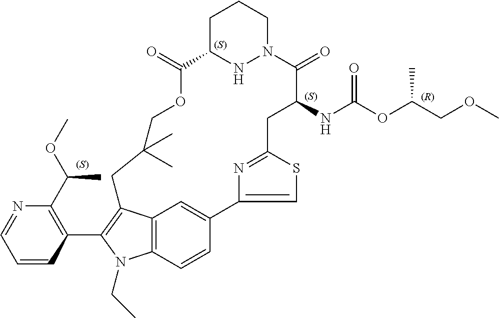 |
| A364 | 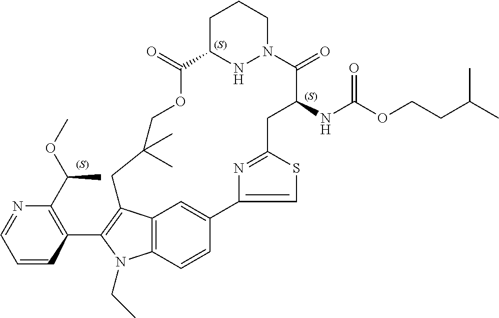 |
| A365 | 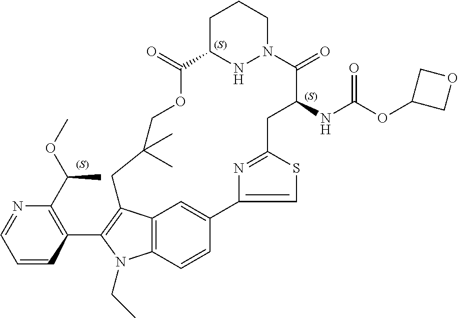 |
| A366 | 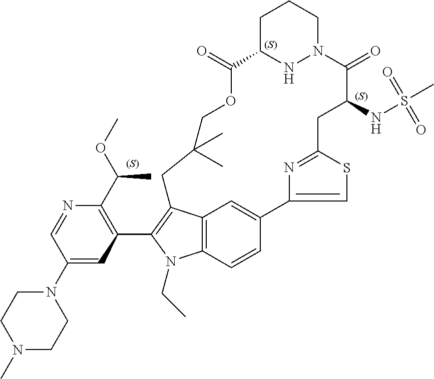 |
| A367 | 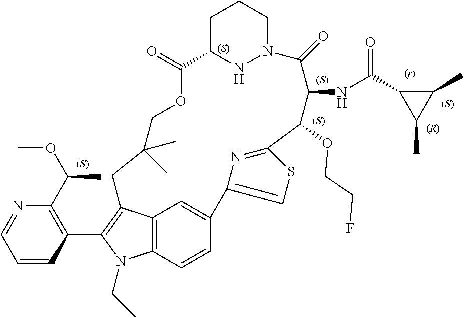 |
| A368 | 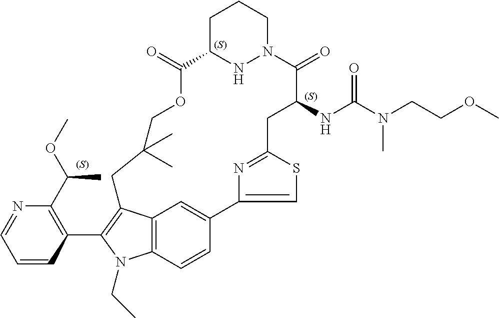 |
| A369 | 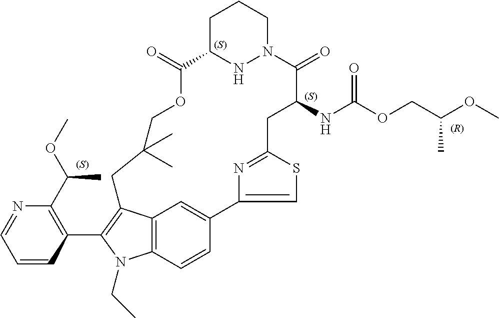 |
| A370 | 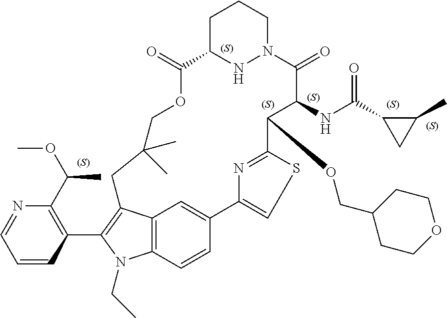 |
| A371 | 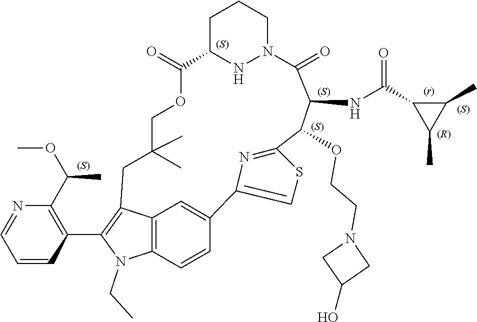 |
| A372 | 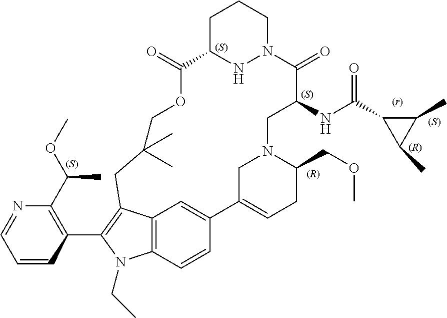 |
| A373 | 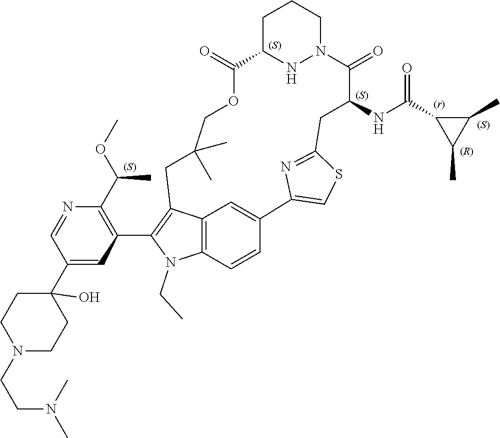 |
| A374 | 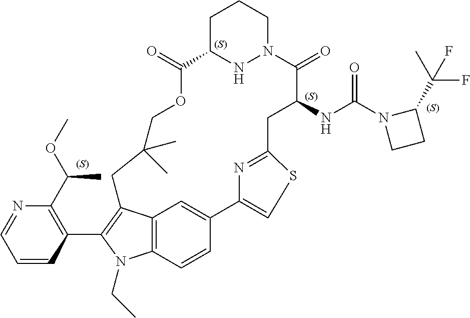 |
| A375 | 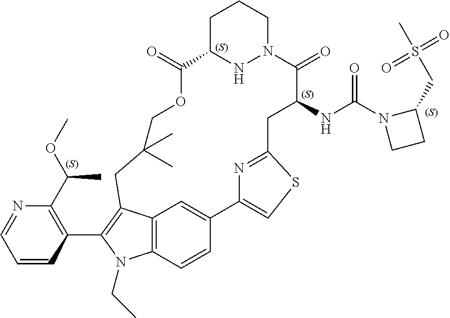 |
| A376 | 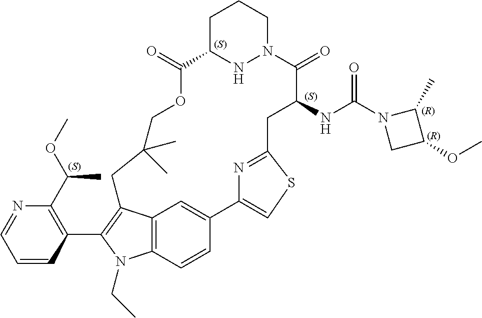 |
| A377 | 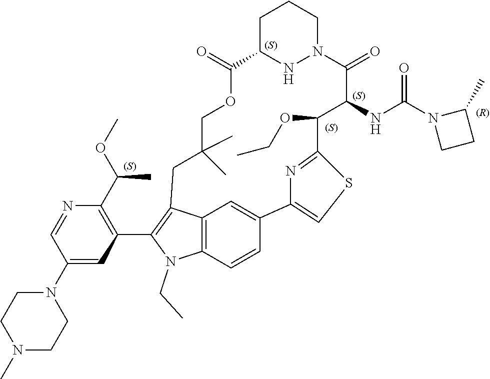 |
| A378 | 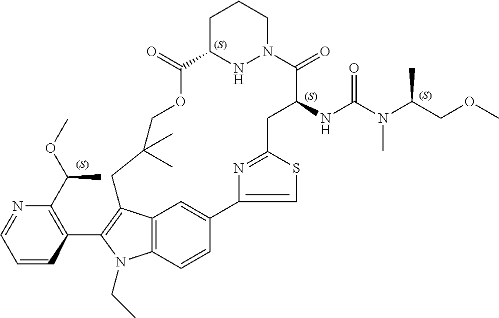 |
| A379 | 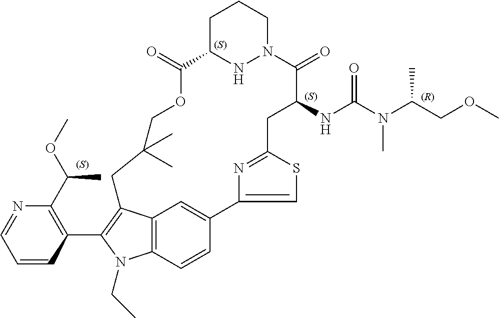 |
| A380 | 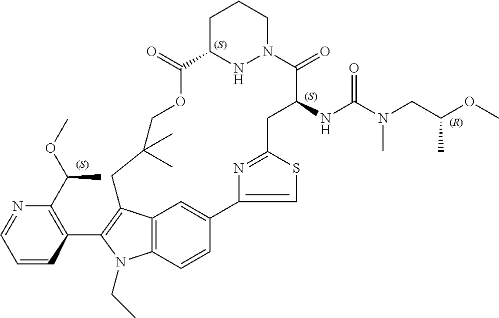 |
| A381 | 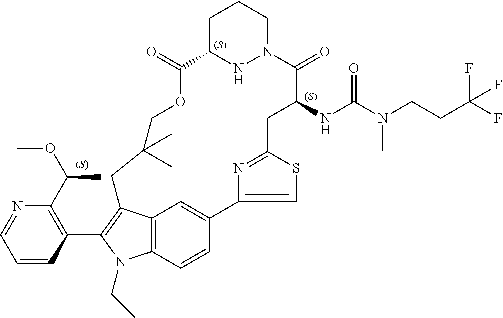 |
| A382 | 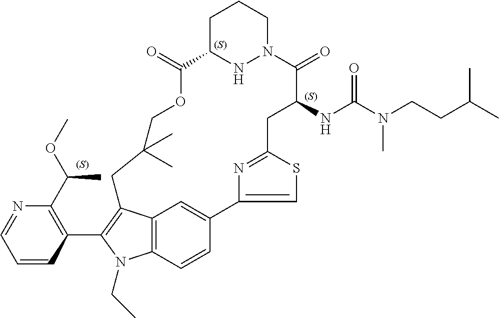 |
| A383 | 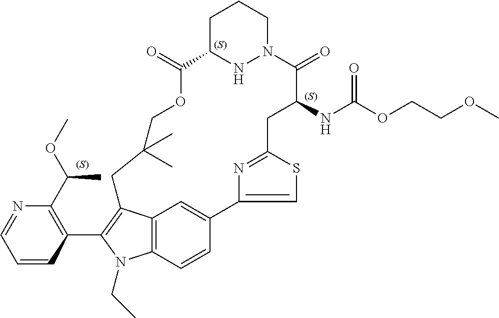 |
| A384 | 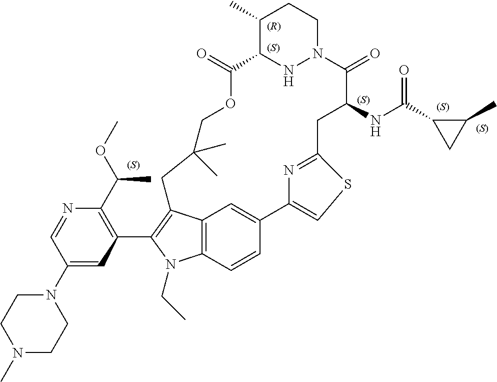 |
| A385 | 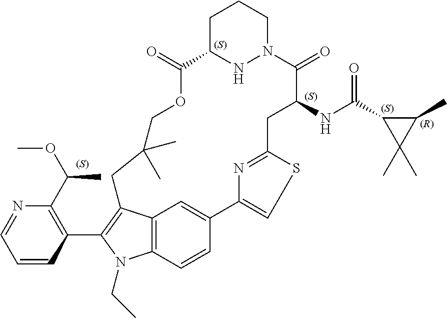 |
| A386 | 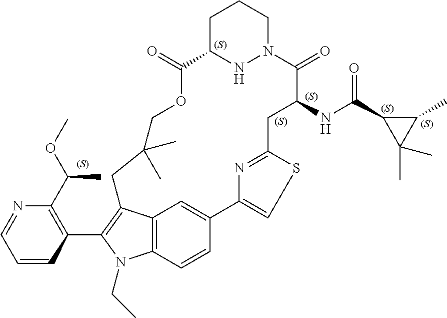 |
| A387 | 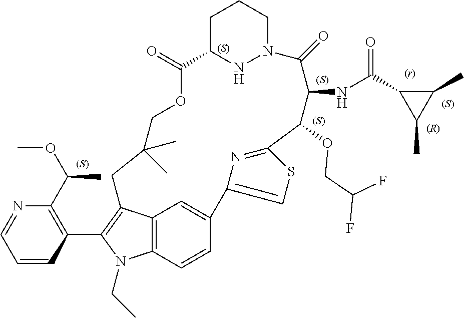 |
| A388 | 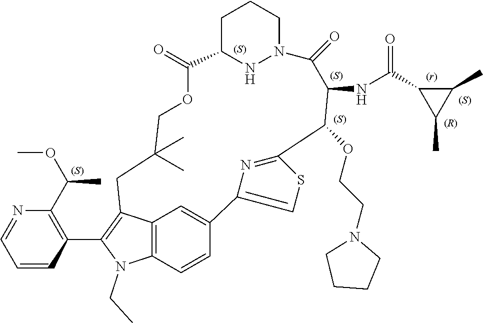 |
| A389 | 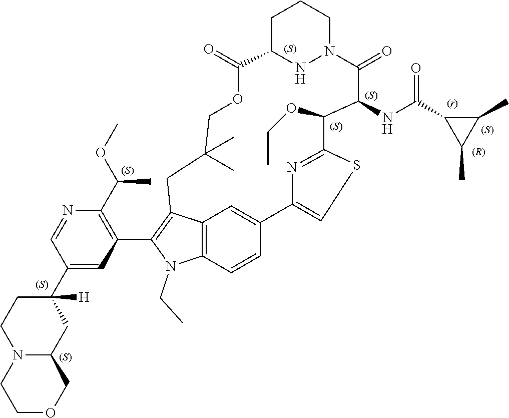 |
| A390 | 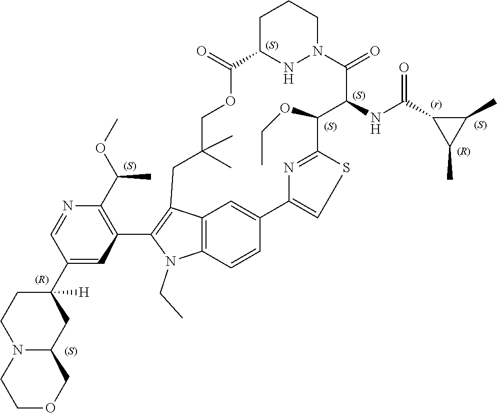 |
| A391 | 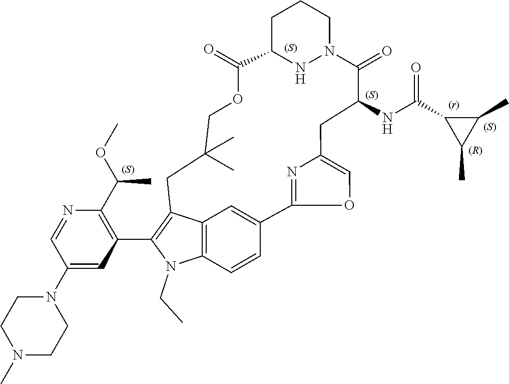 |
| A392 | 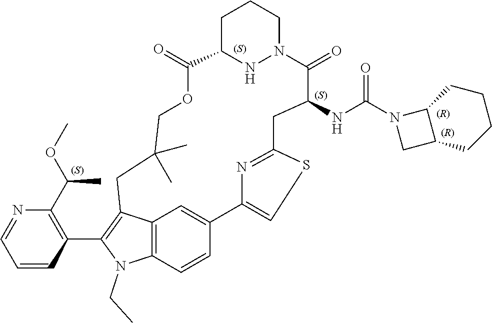 |
| A393 | 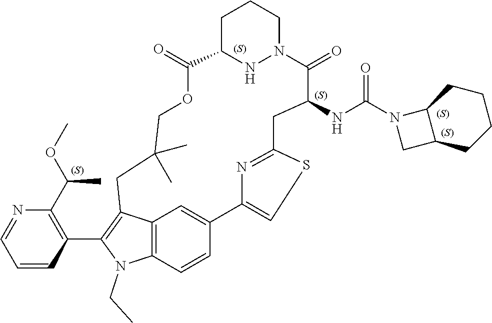 |
| A394 | 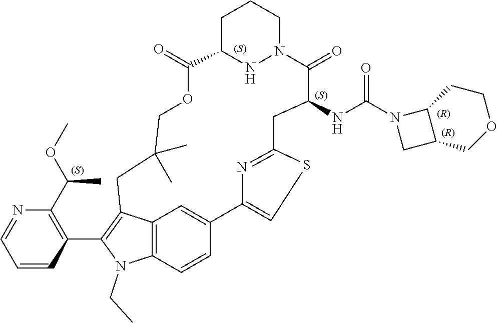 |
| A395 | 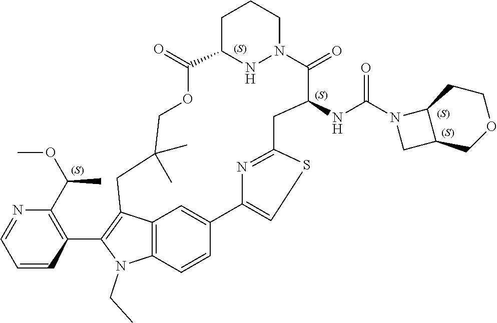 |
| A396 | 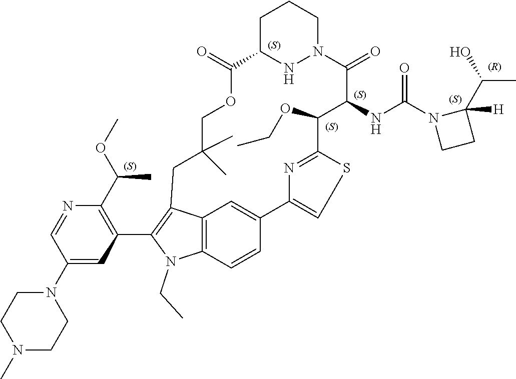 |
| A397 | 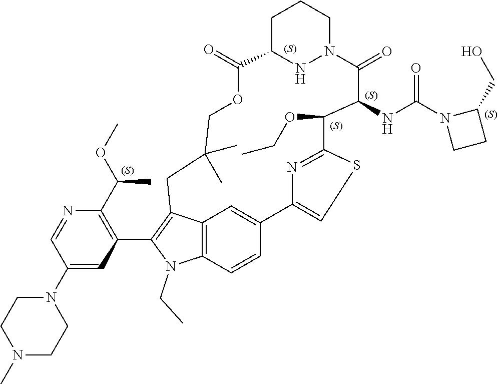 |
| A398 | 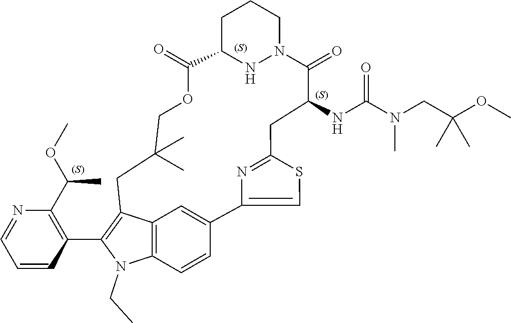 |
| A399 | 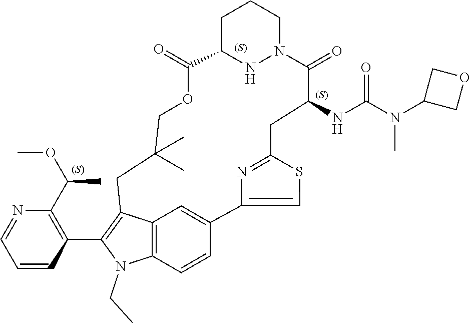 |
| A400 | 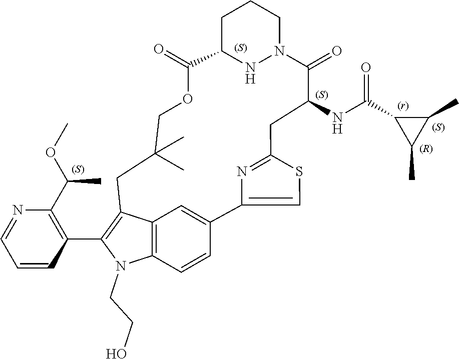 |
| A401 | 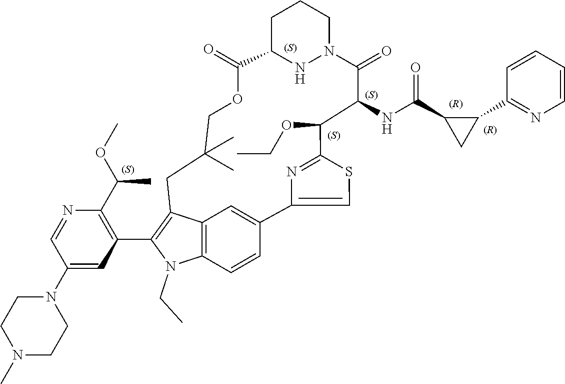 |
| A402 | 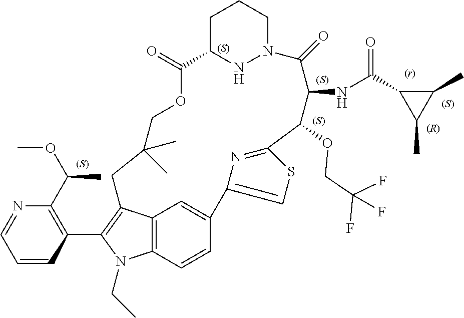 |
| A403 | 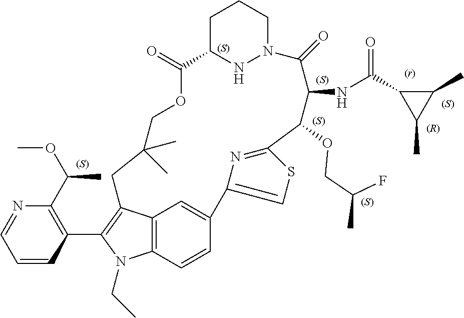 |
| A404 | 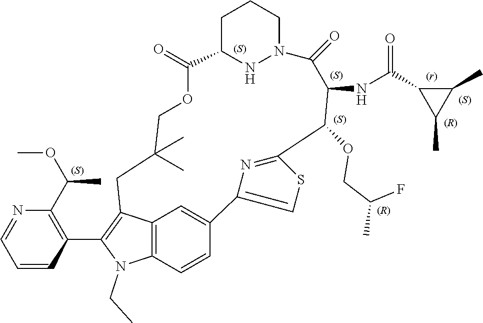 |
| A405 | 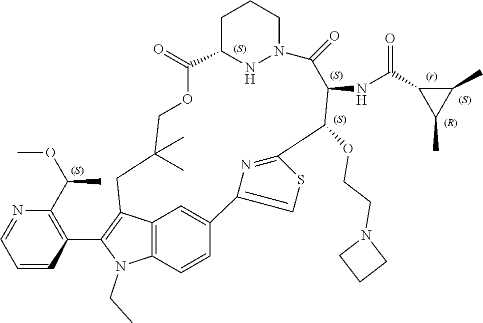 |
| A406 | 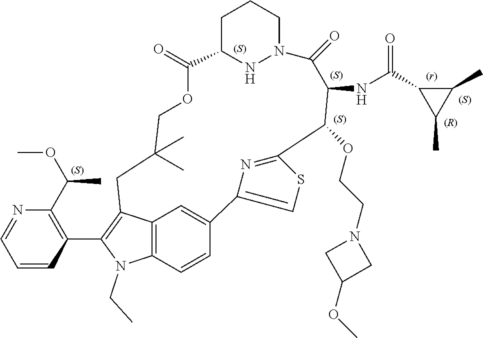 |
| A407 | 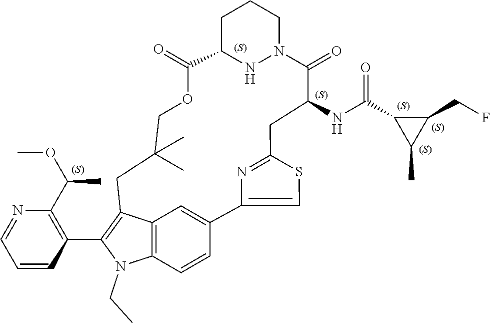 |
| A408 | 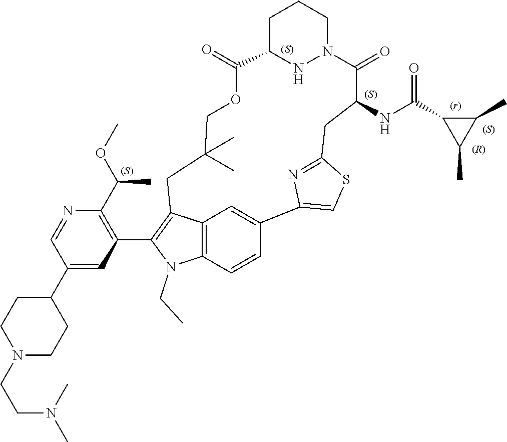 |
| A409 | 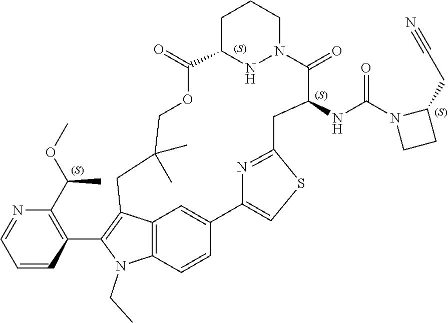 |
| A410 | 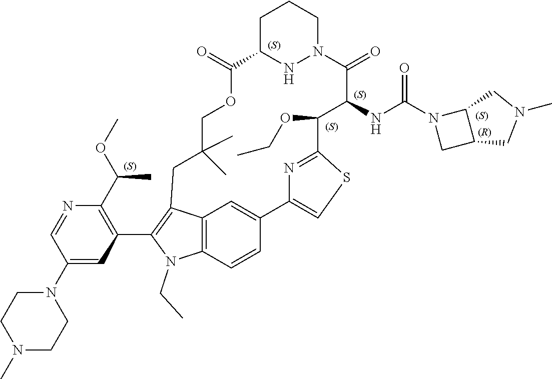 |
| A411 | 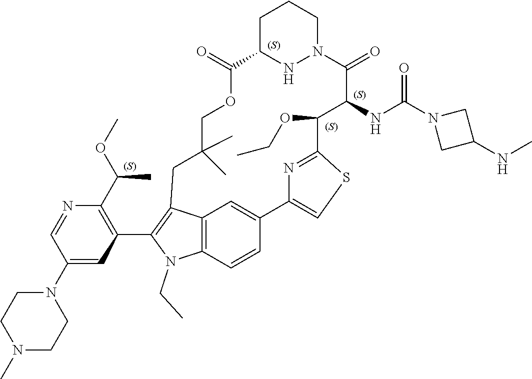 |
| A412 | 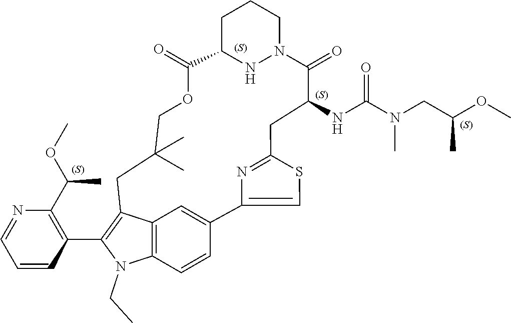 |
| A413 | 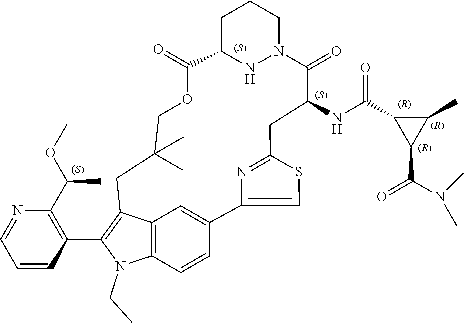 |
| A414 | 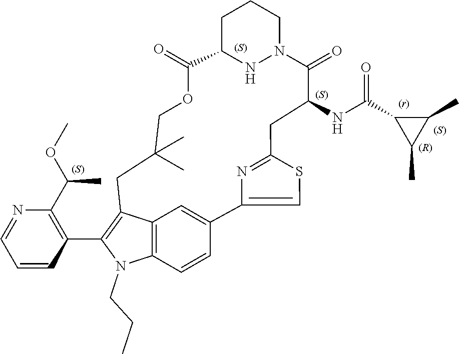 |
| A415 | 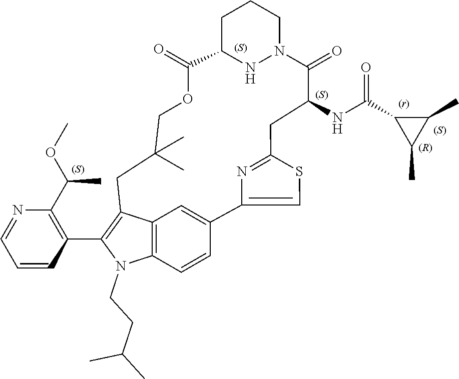 |
| A416 | 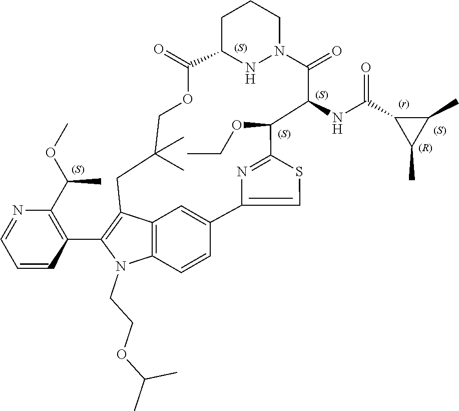 |
| A417 | 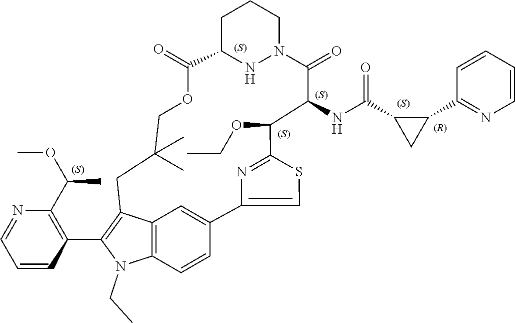 |
| A418 | 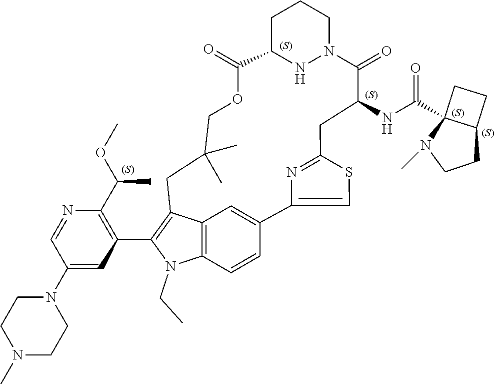 |
| A419 | 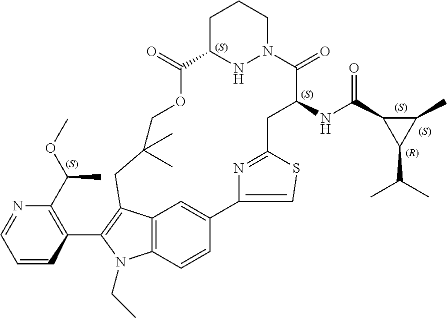 |
| A420 | 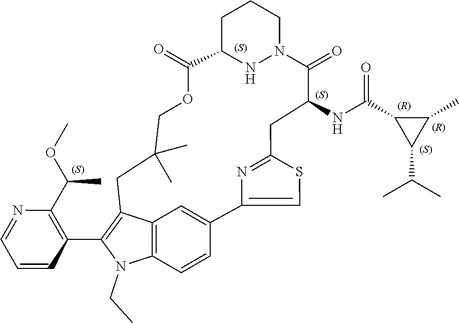 |
| A421 | 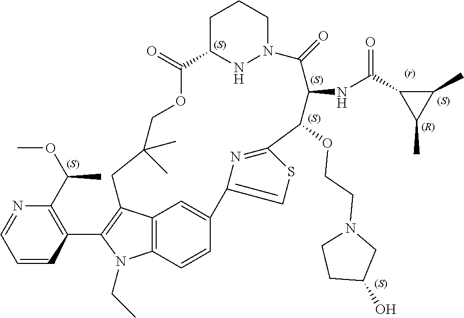 |
| A422 | 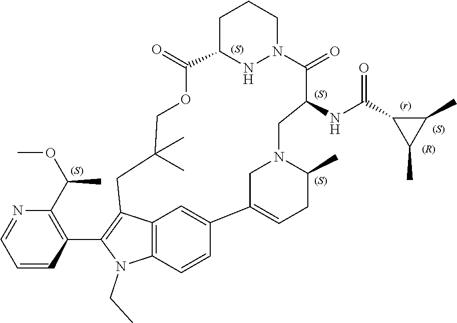 |
| A423 | 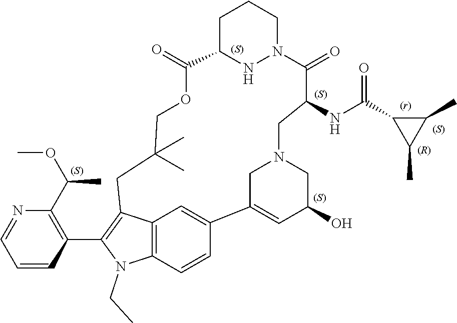 |
| A424 | 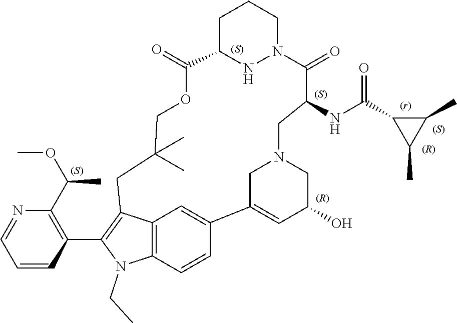 |
| A425 | 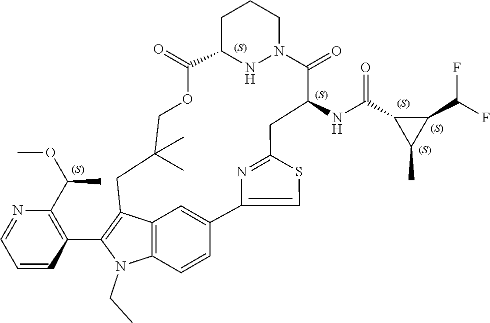 |
| A426 | 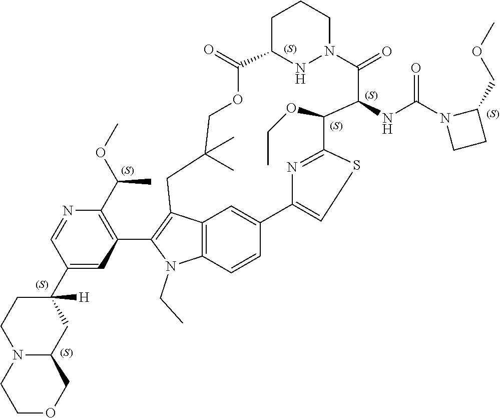 |
| A427 | 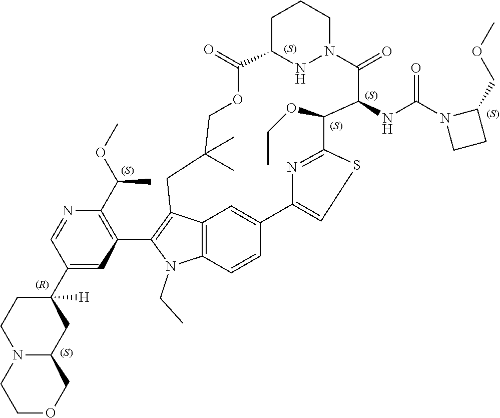 |
| A428 | 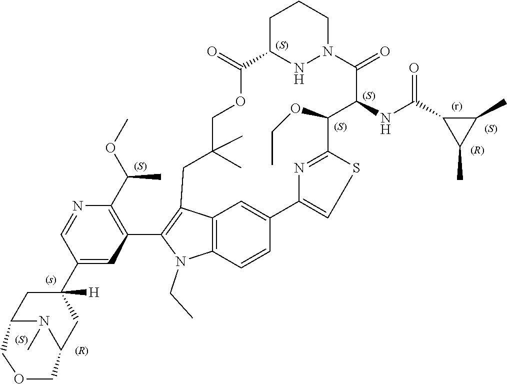 |
| A429 | 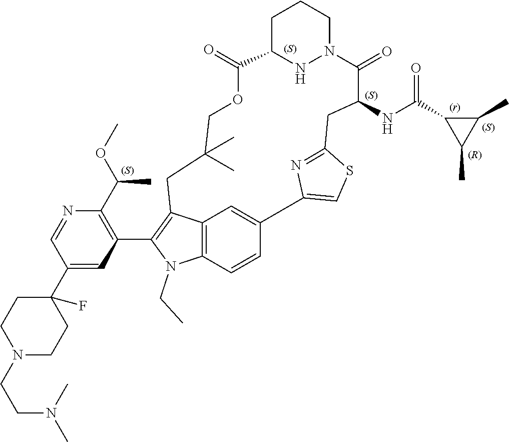 |
| A430 | 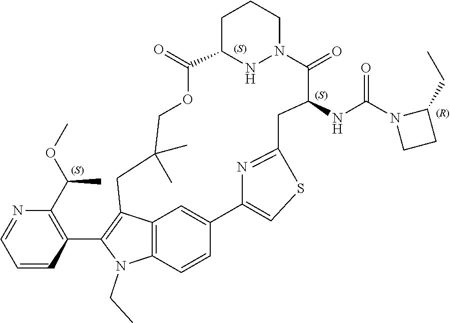 |
| A431 | 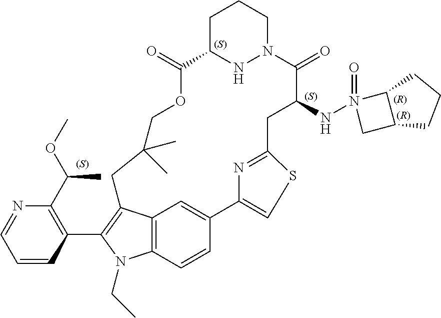 |
| A432 | 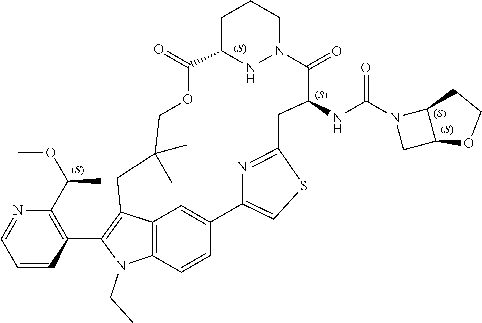 |
| A433 | 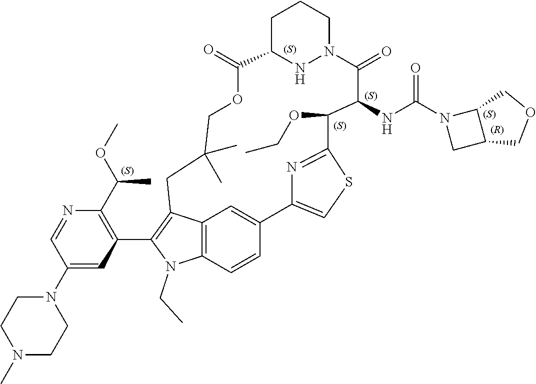 |
| A434 | 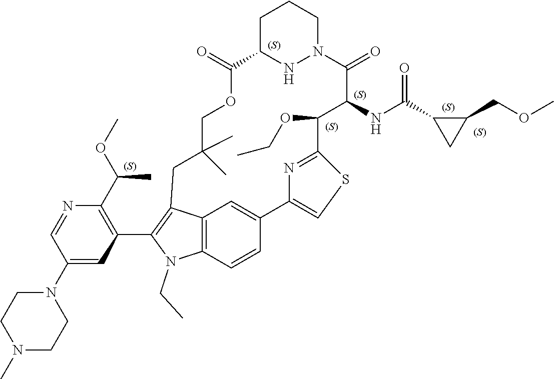 |
| A435 | 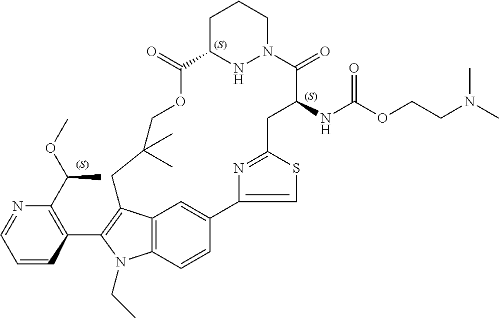 |
| A436 | 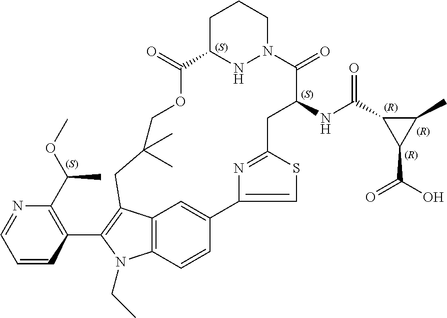 |
| A437 | 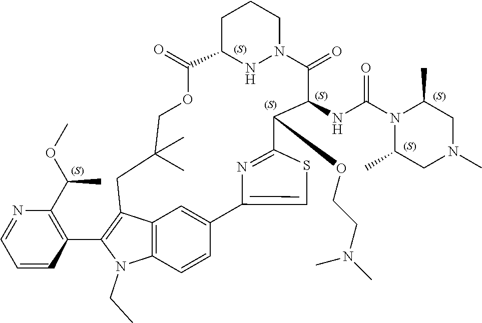 |
| A438 | 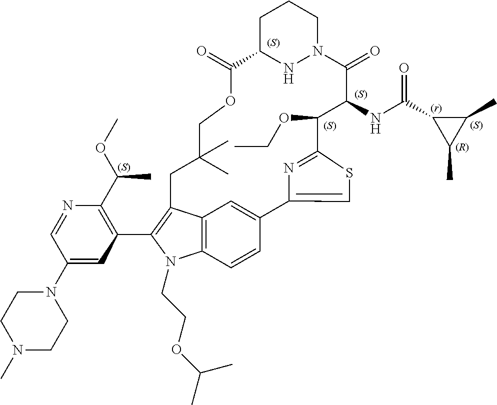 |
| A439 | 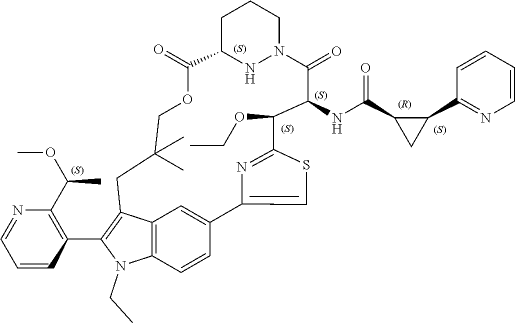 |
| A440 | 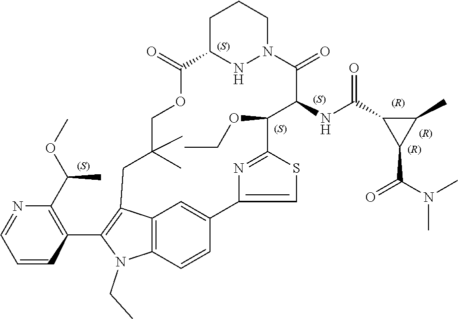 |
| A441 | 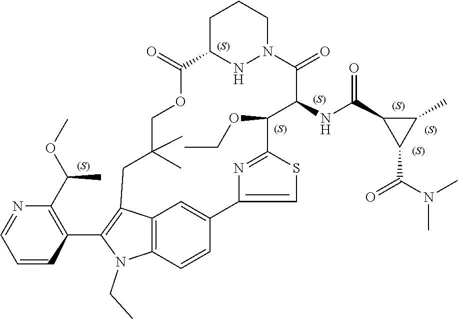 |
| A442 | 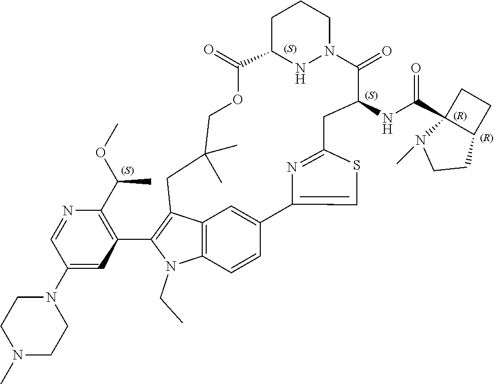 |
| A443 | 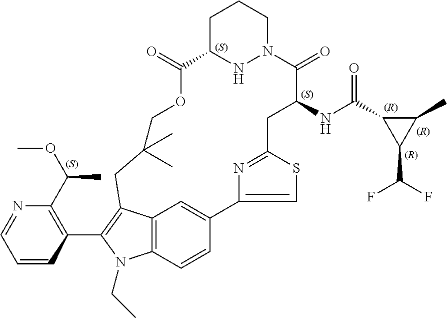 |
| A444 | 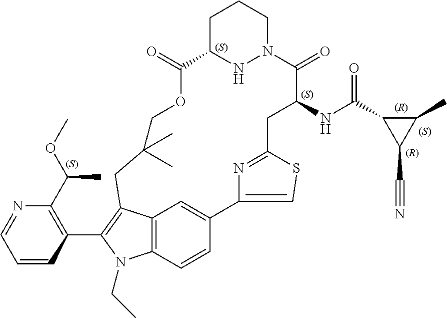 |
| A445 | 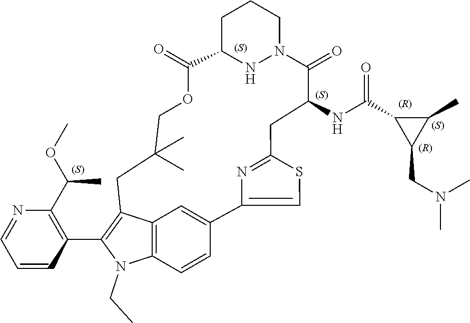 |
| A446 | 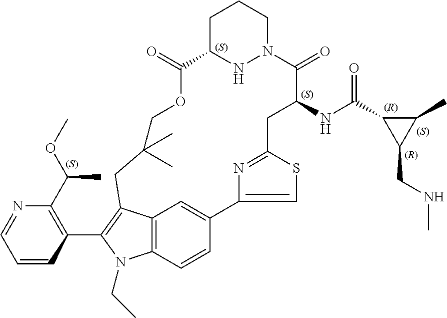 |
| A447 | 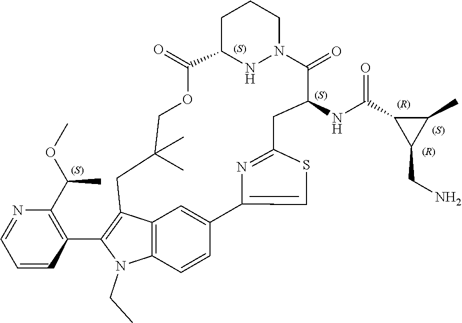 |
| A448 | 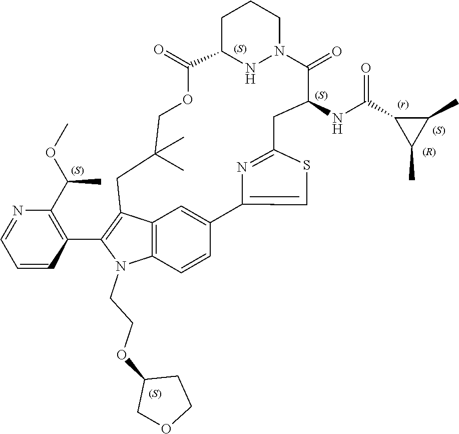 |
| A449 | 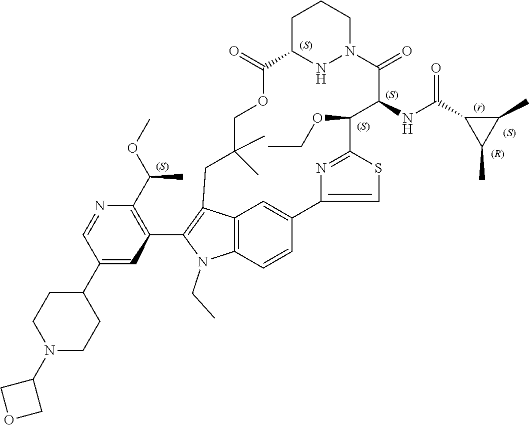 |
| A450 | 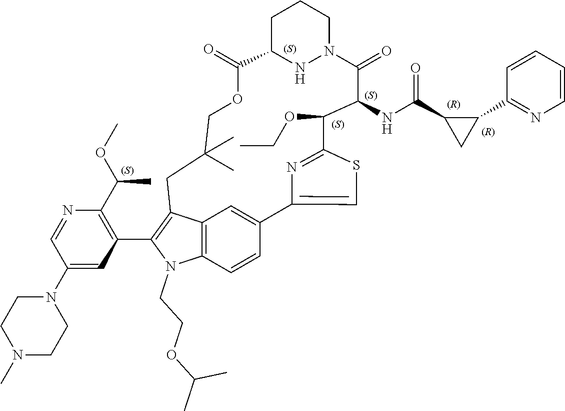 |
| A451 | 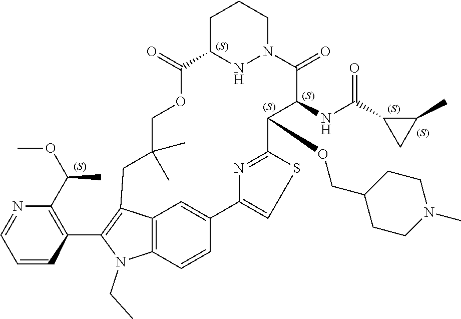 |
| A452 | 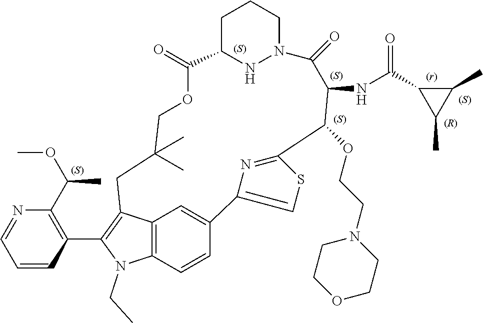 |
| A453 | 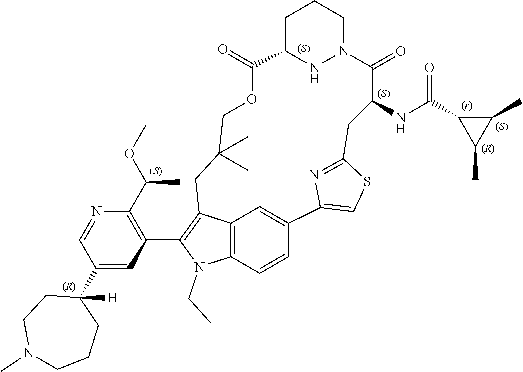 |
| A454 | 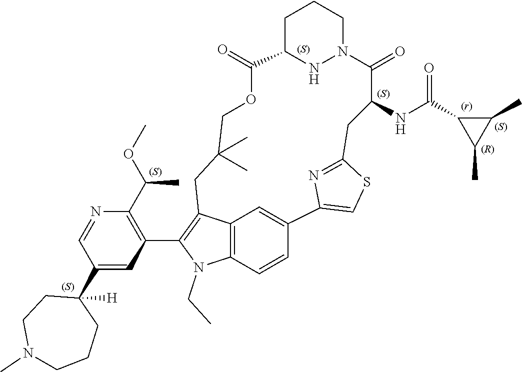 |
| A455 | 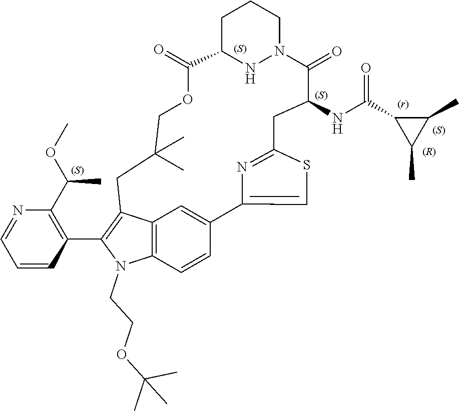 |
| A456 | 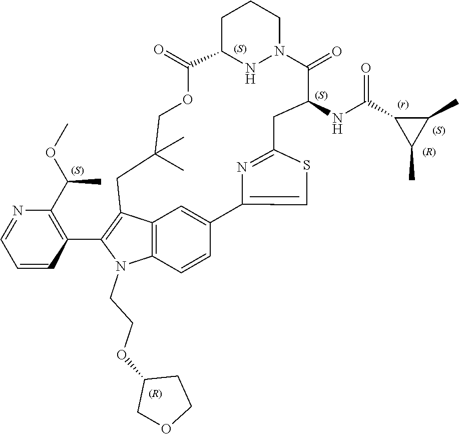 |
| A457 | 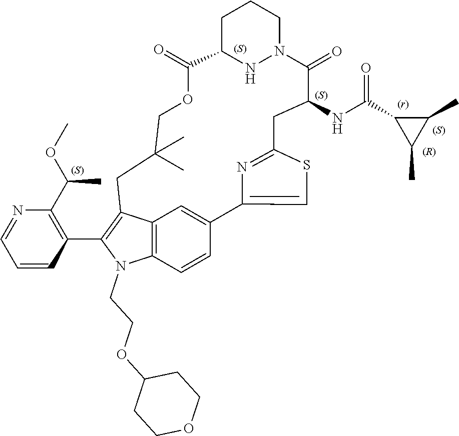 |
| A458 | 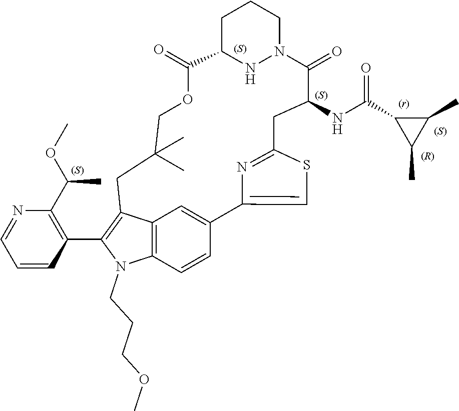 |
| A459 | 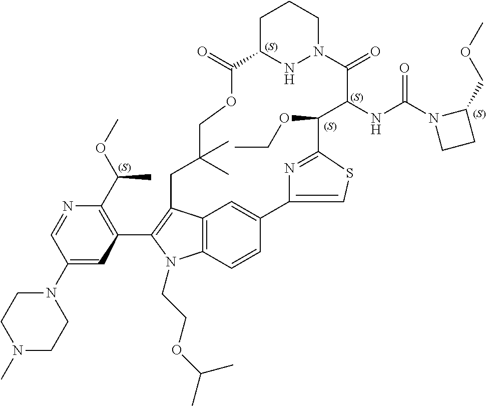 |
| A460 | 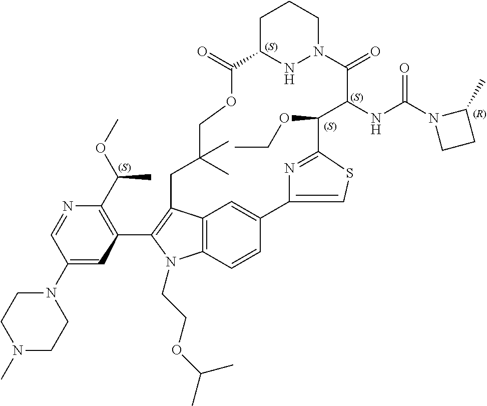 |
| A461 | 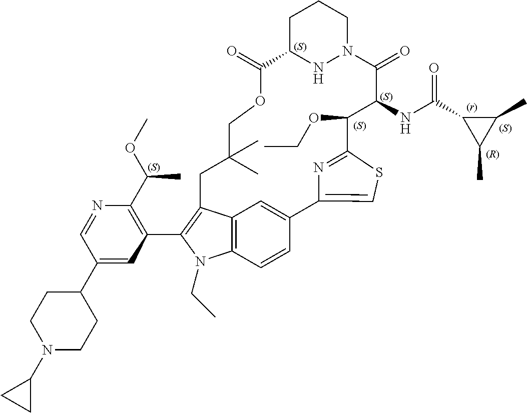 |
| A462 | 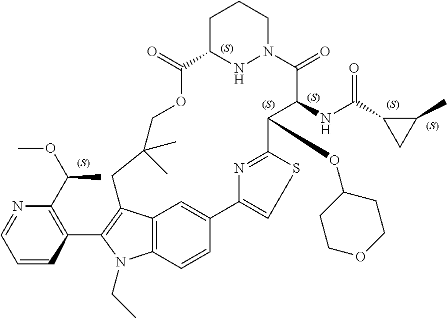 |
| A463 | 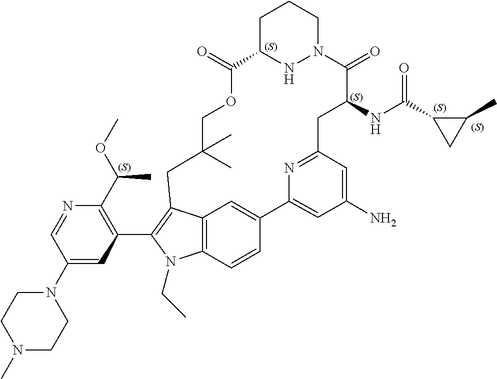 |
| A464 | 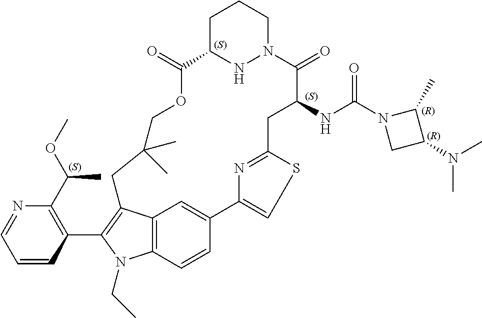 |
| A465 | 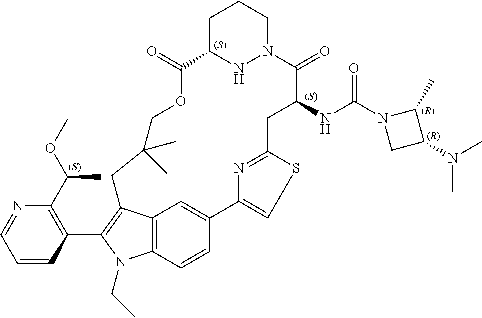 |
| A466 | 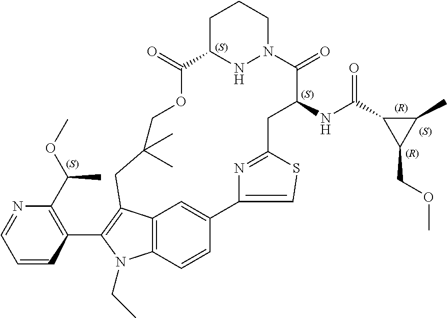 |
| A467 | 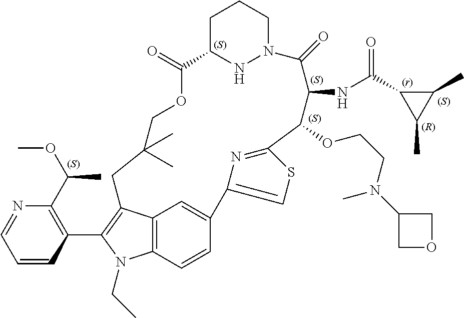 |
| A468 | 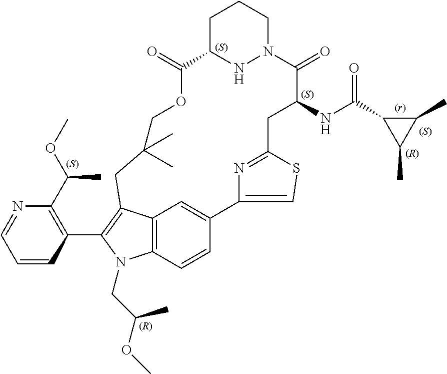 |
| A469 | 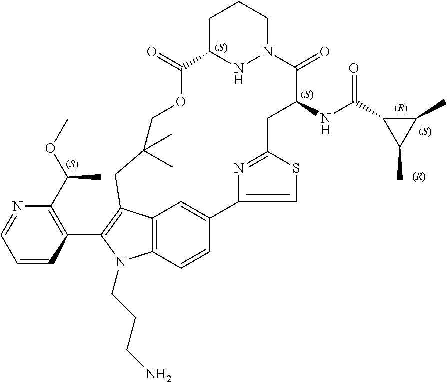 |
| A470 | 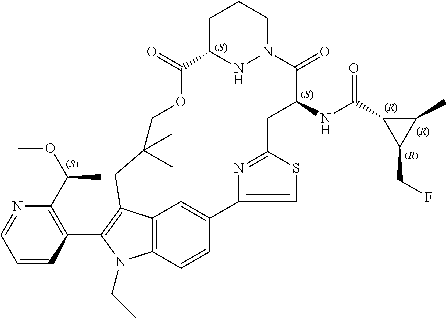 |
| A471 | 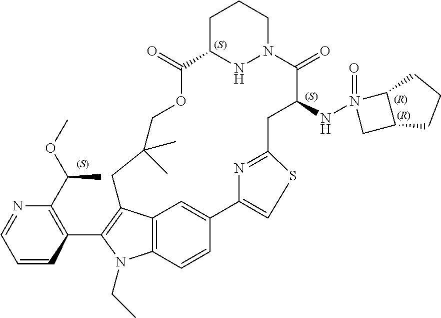 |
| A472 | 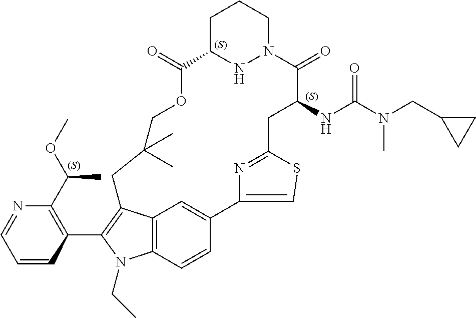 |
| A473 | 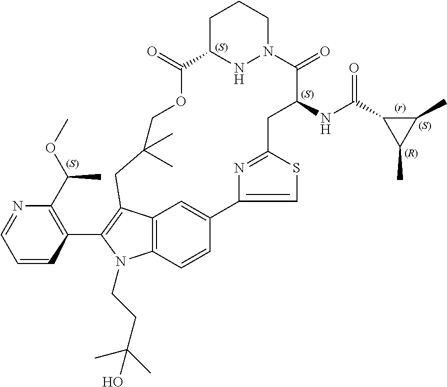 |
| A474 | 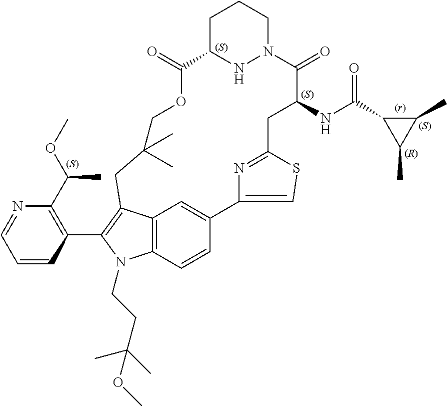 |
| A475 | 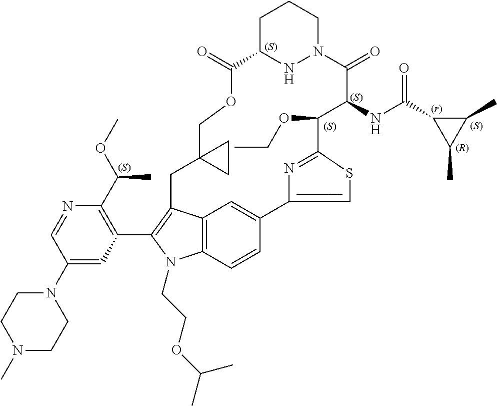 |
| A476 | 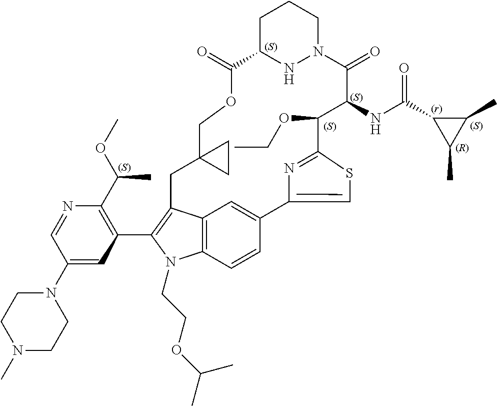 |
| A477 | 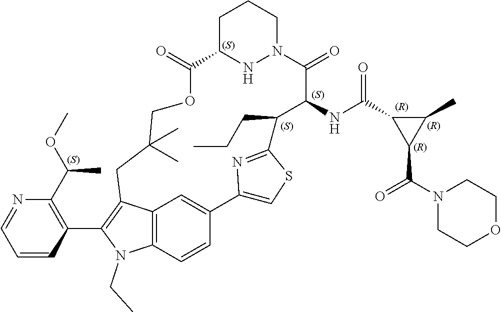 |
| A478 | 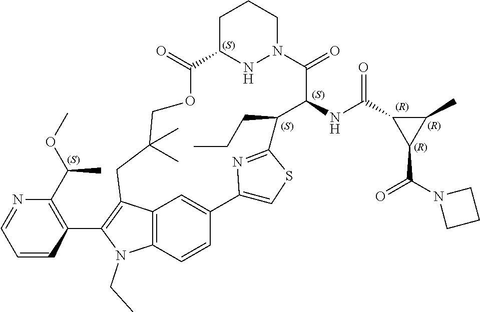 |
| A479 | 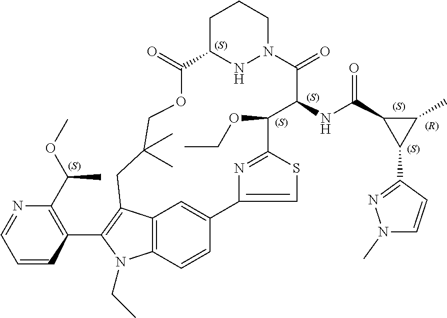 |
| A480 | 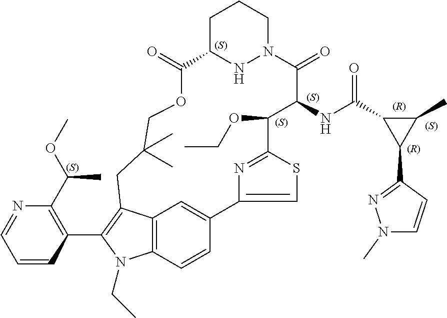 |
| A481 | 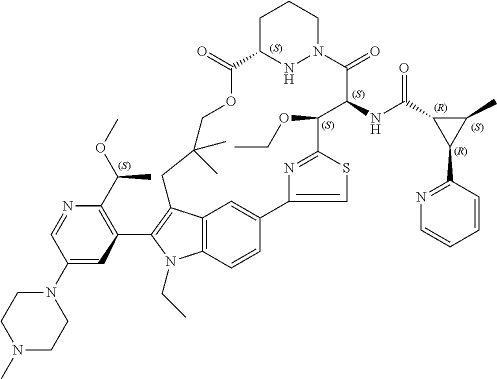 |
| A482 | 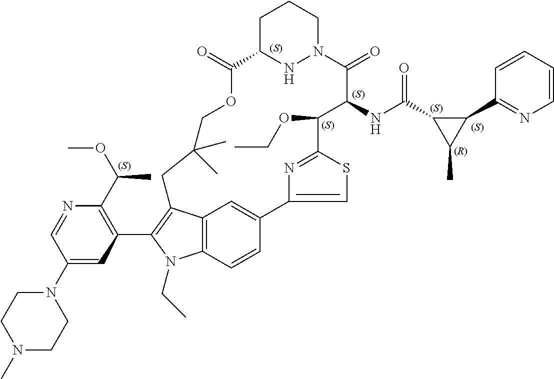 |
| A483 | 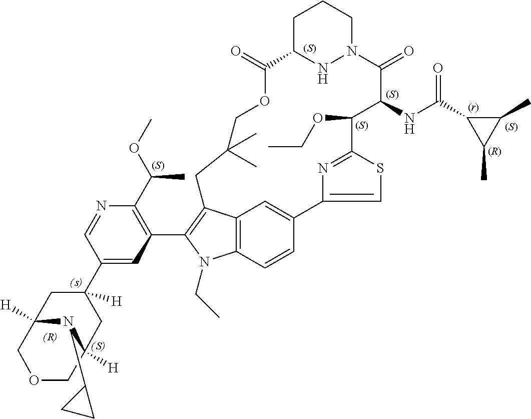 |
| A484 | 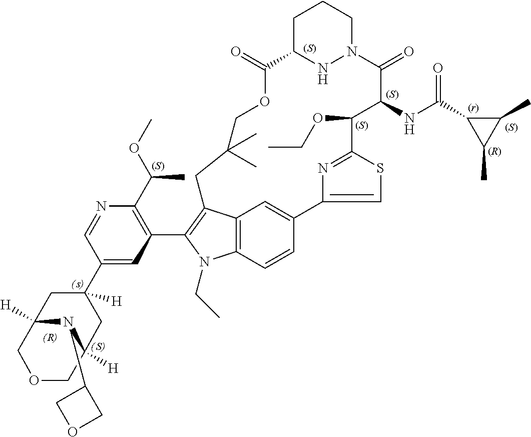 |
| A485 | 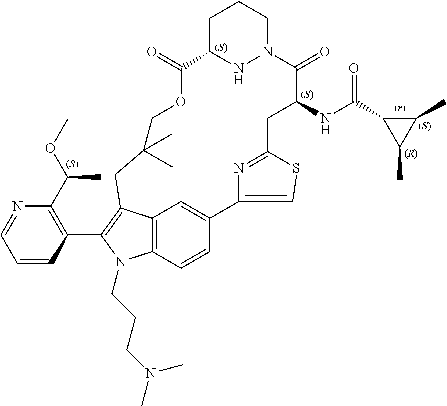 |
| A486 | 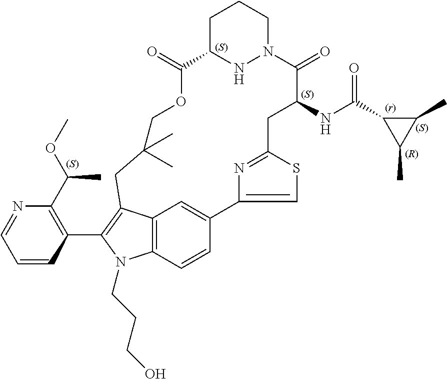 |
| A487 | 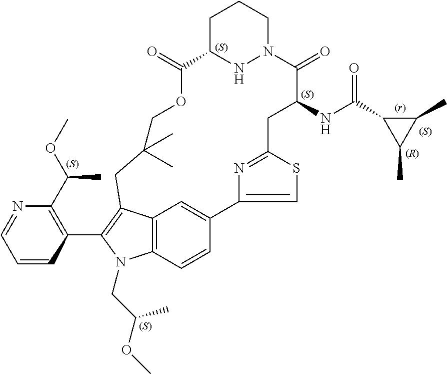 |
| A488 | 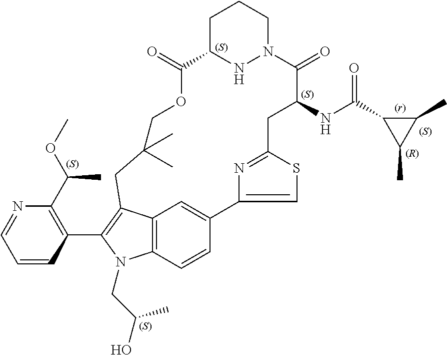 |
| A489 | 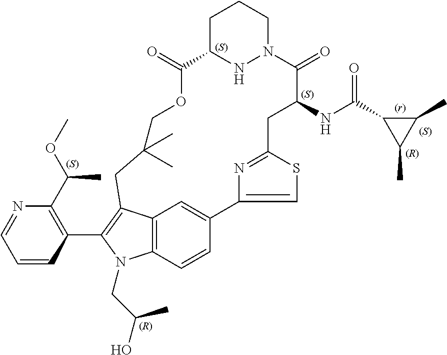 |
| A490 | 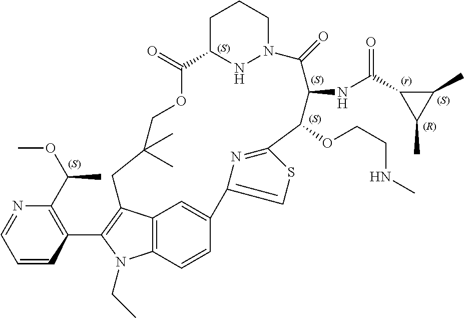 |
| A491 | 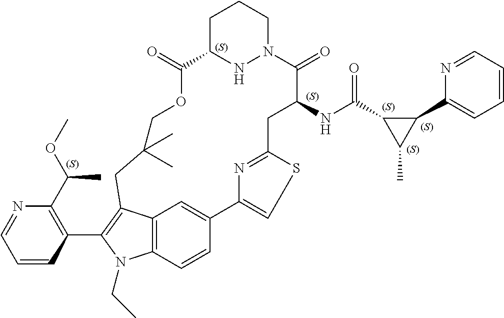 |
| A492 | 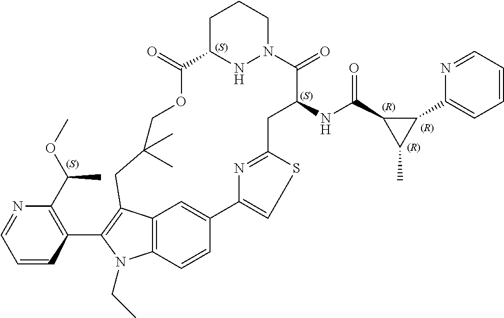 |
| A493 | 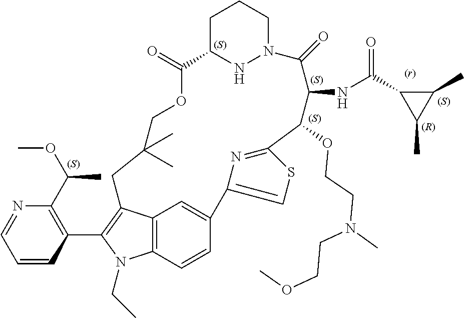 |
| A494 | 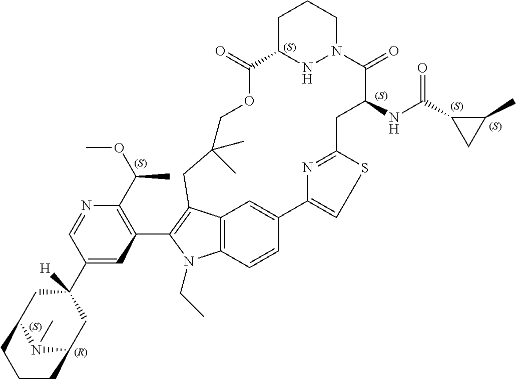 |
| A495 | 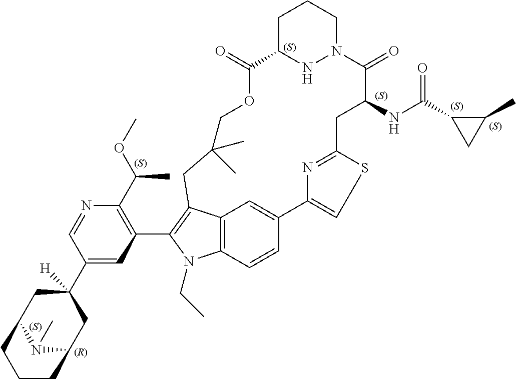 |
| A496 | 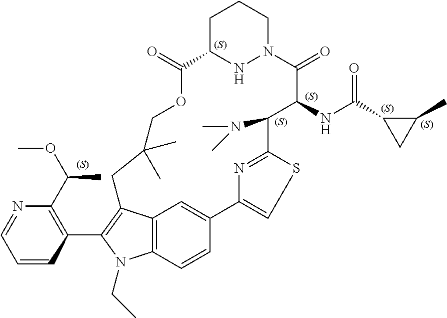 |
| A497 | 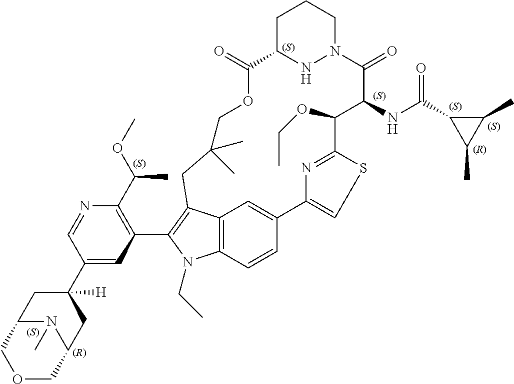 |
| A498 | 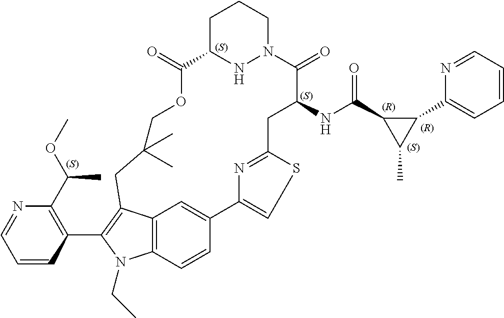 |
| A499 | 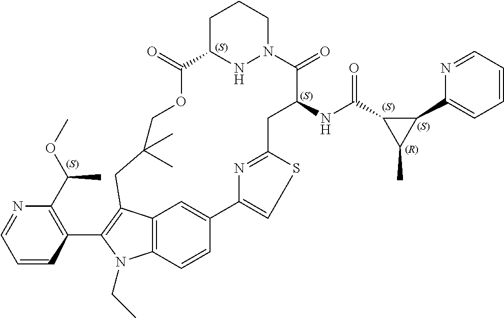 |
| A500 | 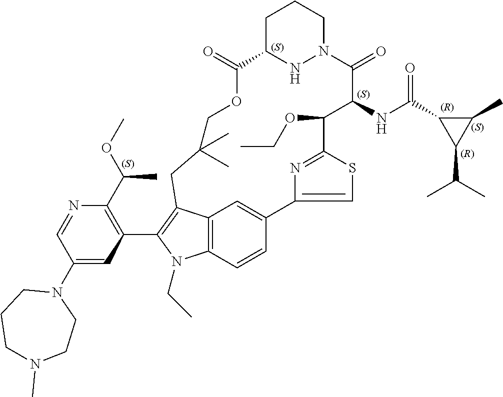 |
| A501 | 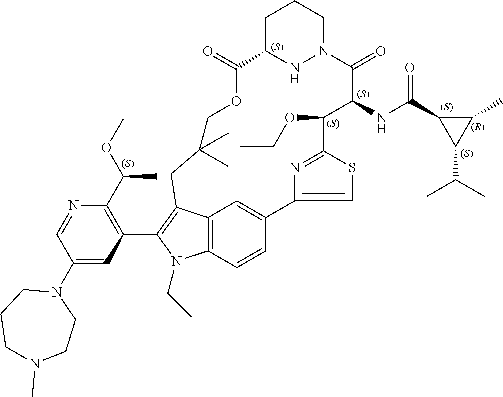 |
| A502 | 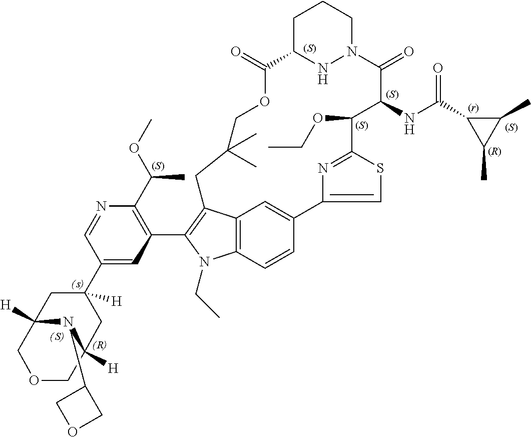 |
| A503 | 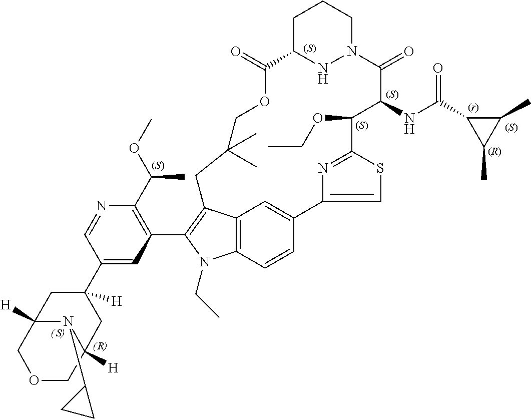 |
| A504 | 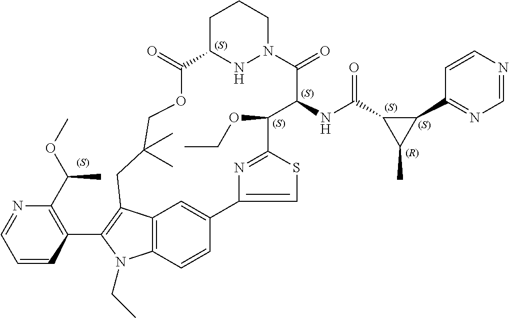 |
| A505 | 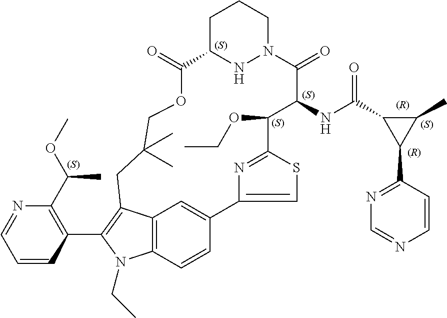 |
| A506 | 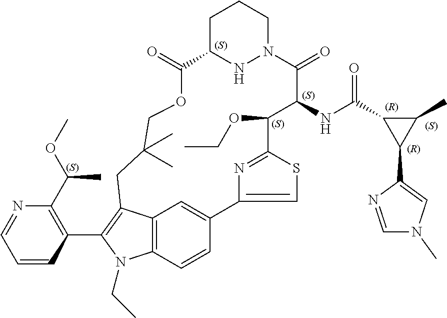 |
| A507 | 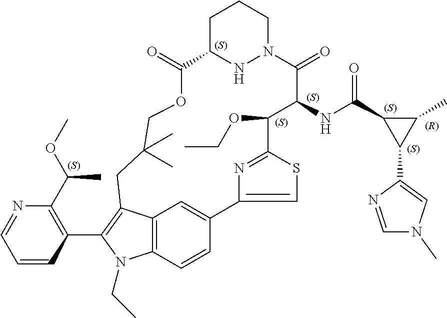 |
| A508 | 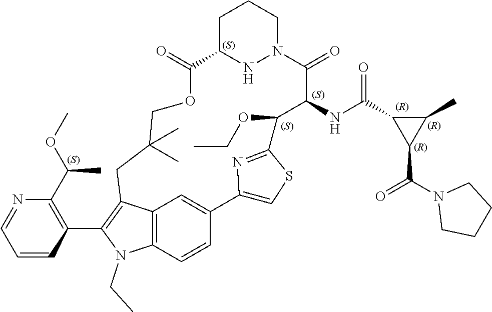 |
| A509 | 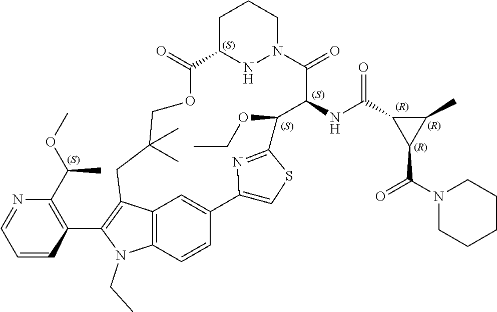 |
| A510 | 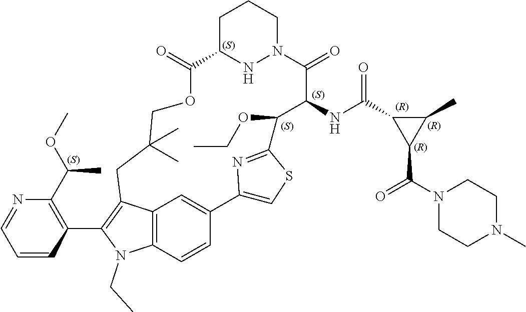 |
| A511 | 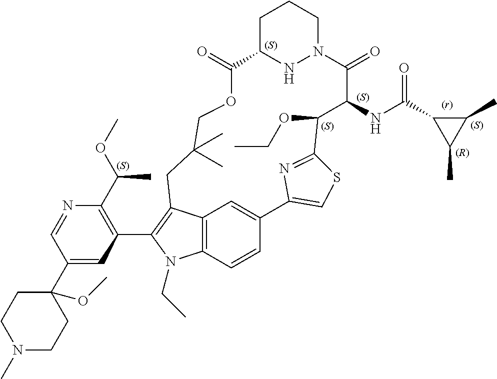 |
| A512 | 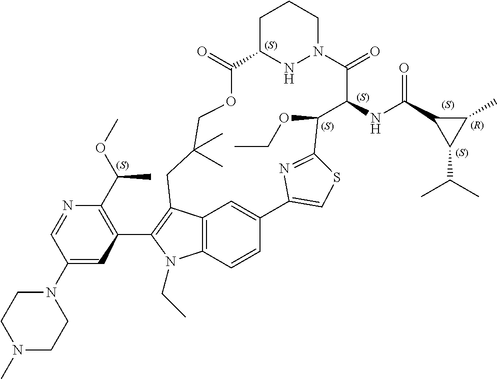 |
| A513 | 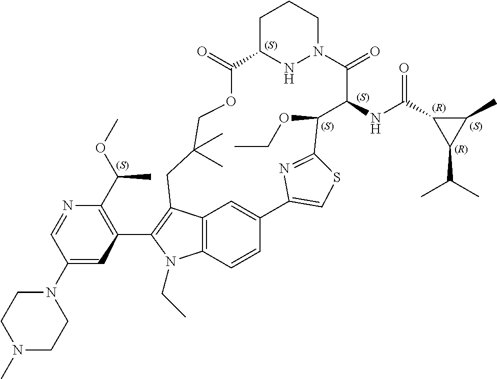 |
| A514 | 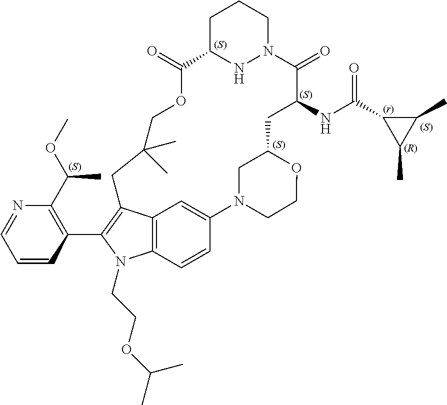 |
| A515 | 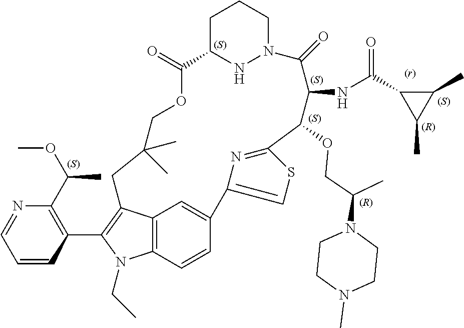 |
| A516 | 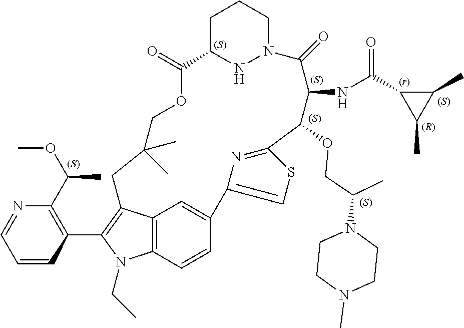 |
| A517 | 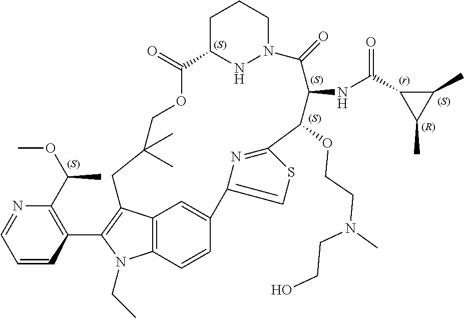 |
| A518 | 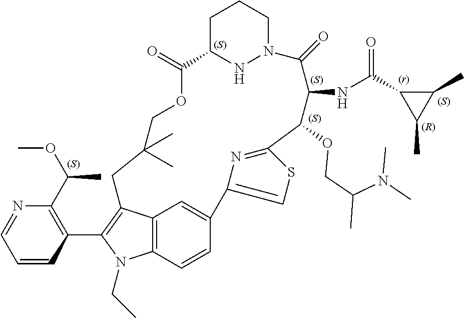 |
| A519 | 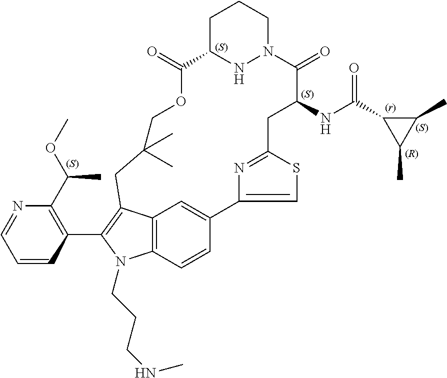 |
| A520 | 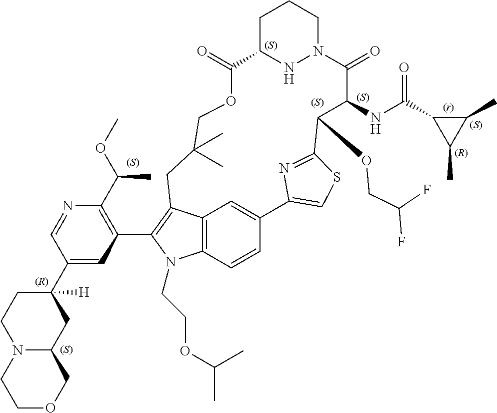 |
| A521 | 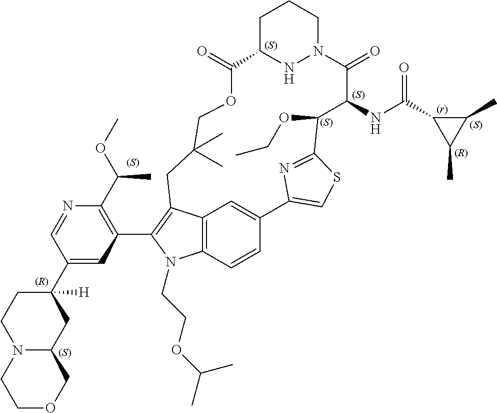 |
| A522 | 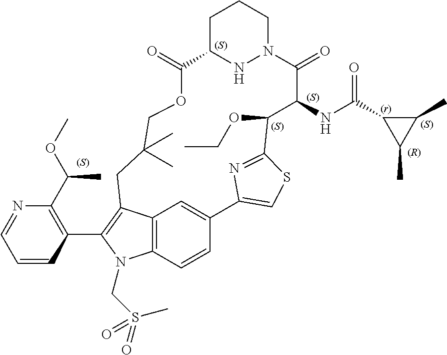 |
| A523 | 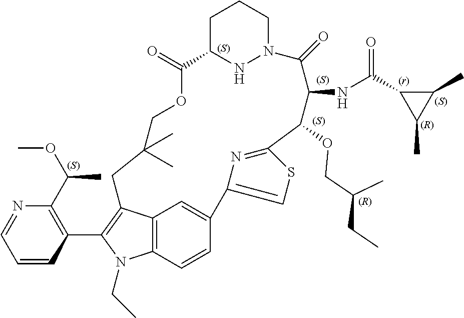 |
| A524 | 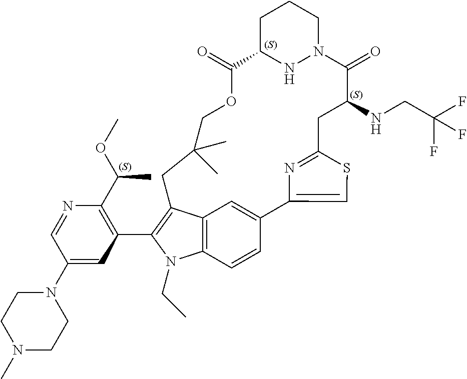 |
| A525 | 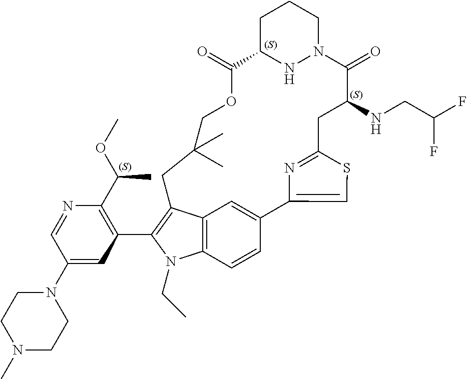 |
| A526 | 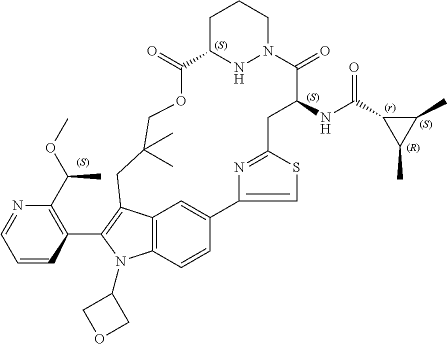 |
| A527 | 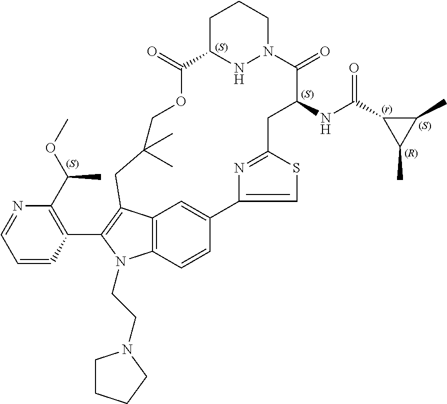 |
| A528 | 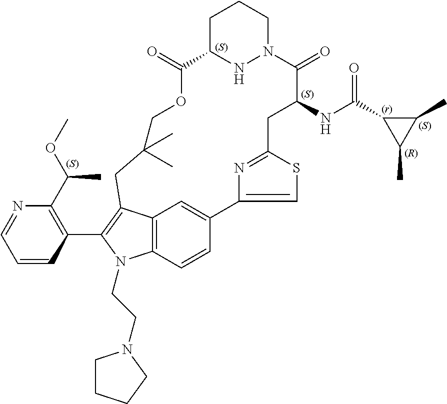 |
| A529 | 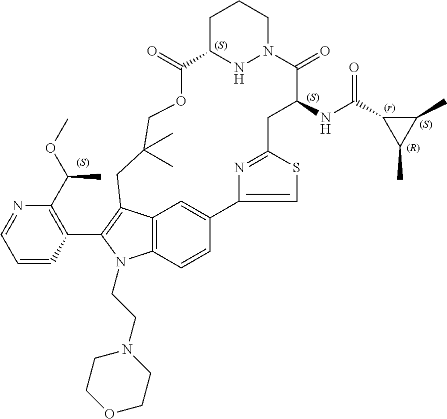 |
| A530 | 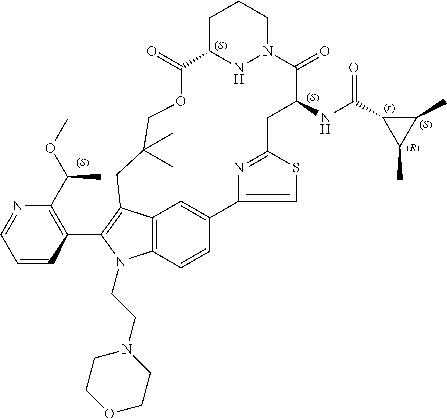 |
| A531 | 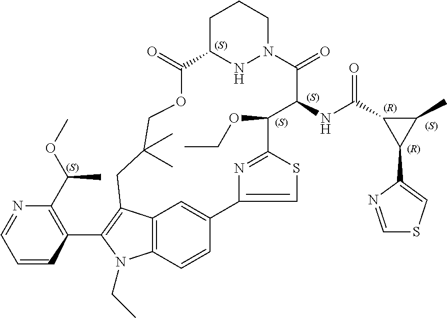 |
| A532 | 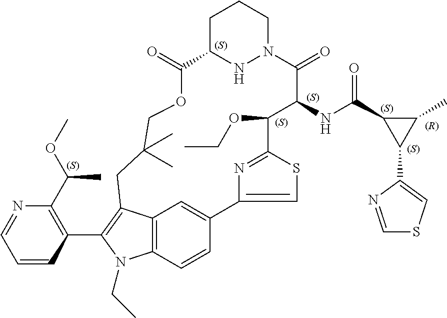 |
| A533 | 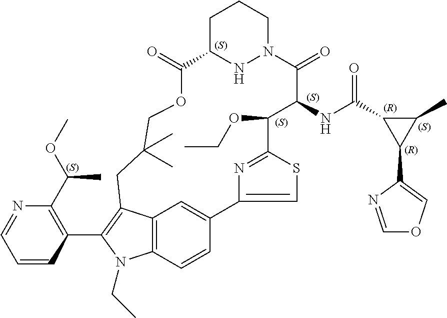 |
| A534 | 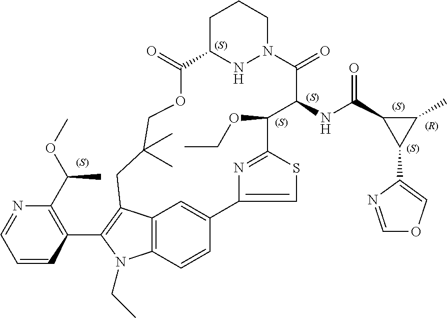 |
| A535 | 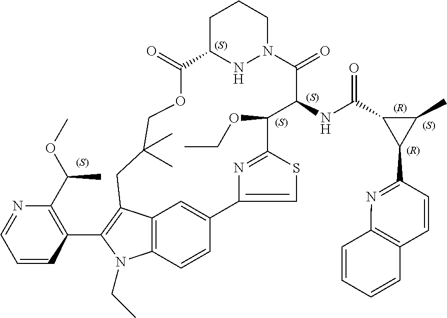 |
| A536 | 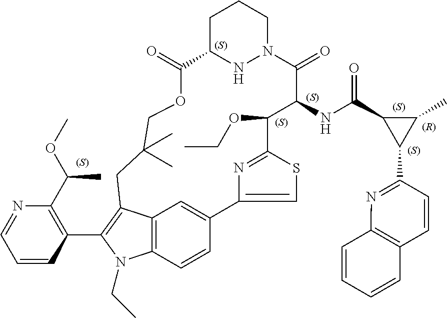 |
| A537 | 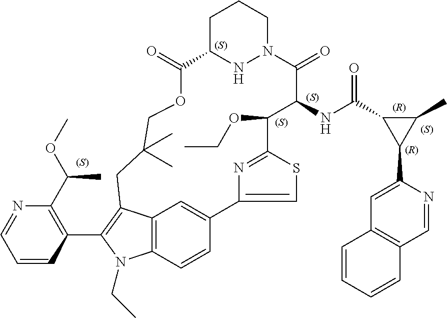 |
| A538 | 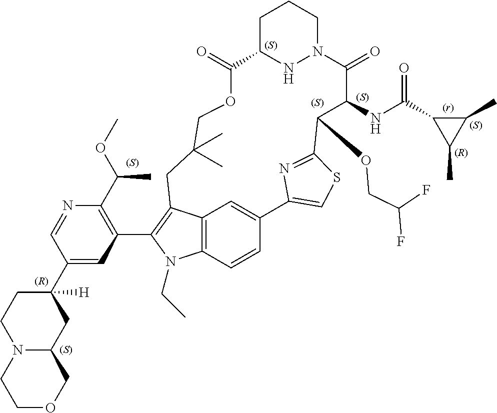 |
| A539 | 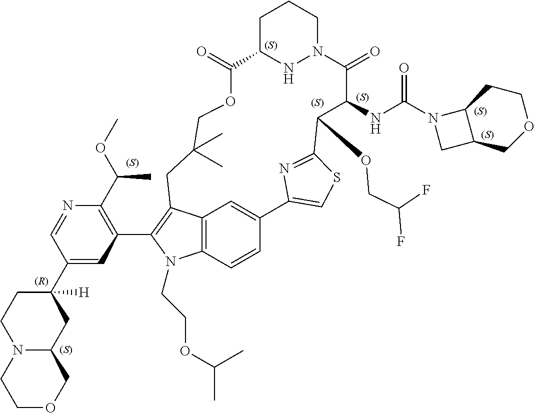 |
| A540 | 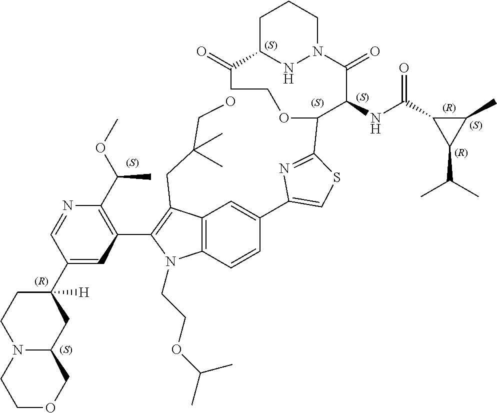 |
| A541 | 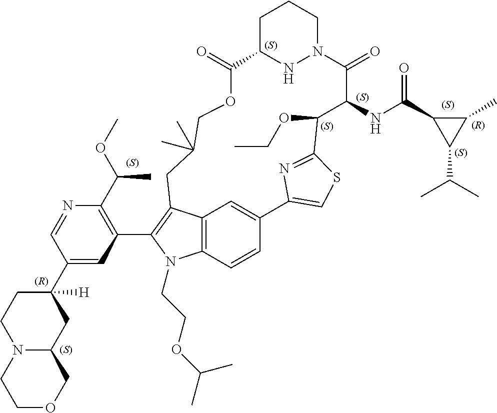 |
| A542 | 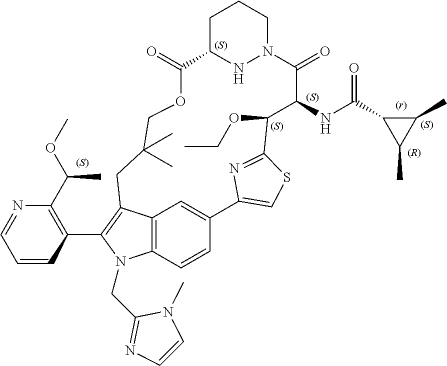 |
| A543 | 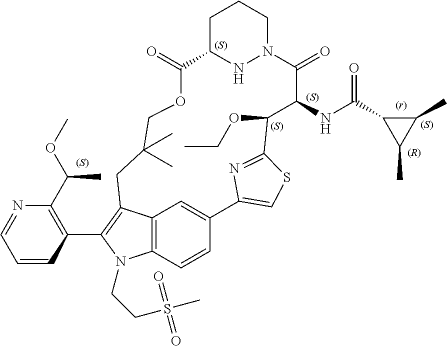 |
| A544 | 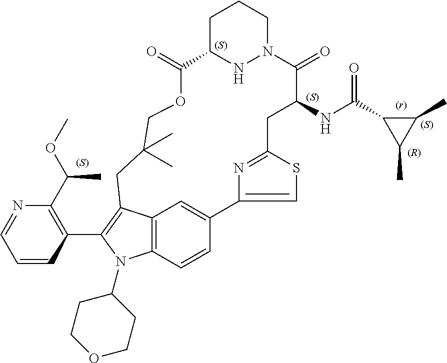 |
| A545 | 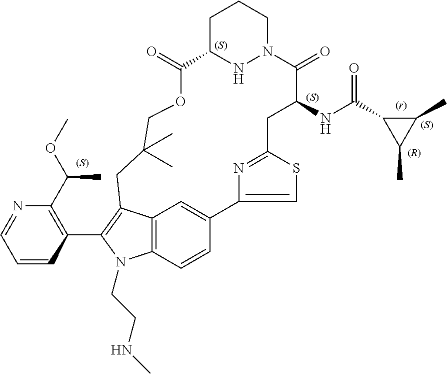 |
| A546 | 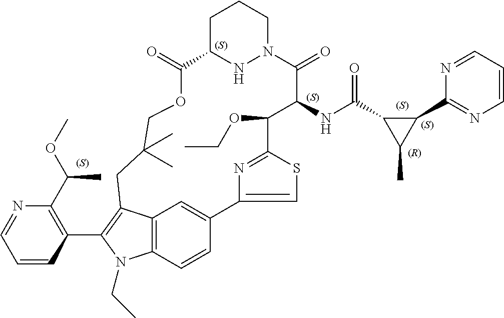 |
| A547 | 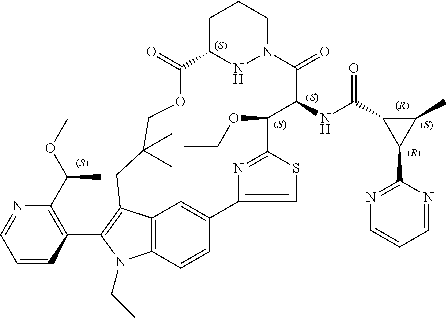 |
| A548 | 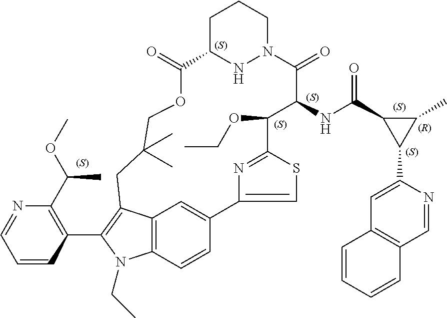 |
| A549 | 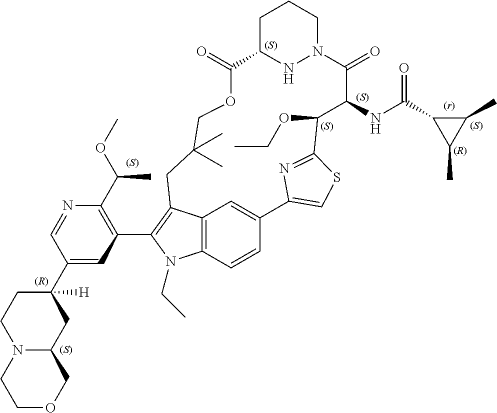 |
| A550 | 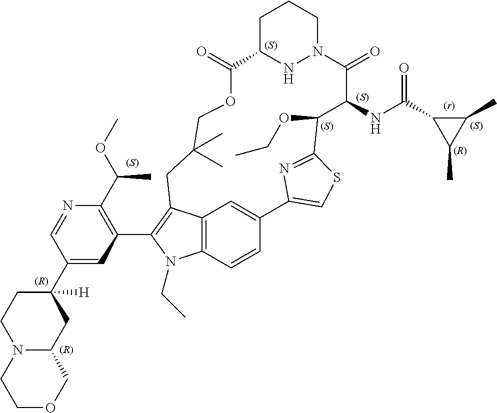 |
| A551 | 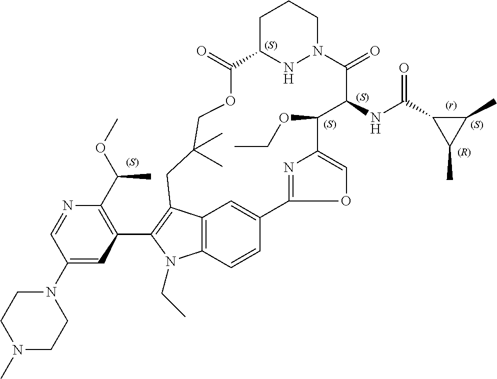 |
| A552 | 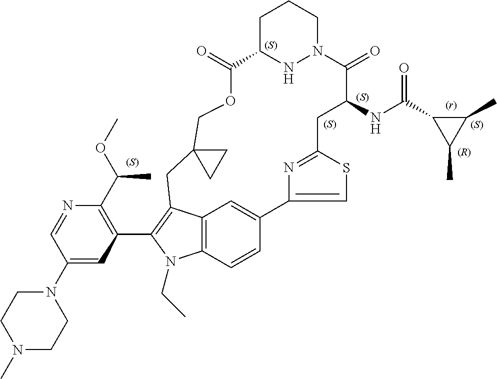 |
| A553 | 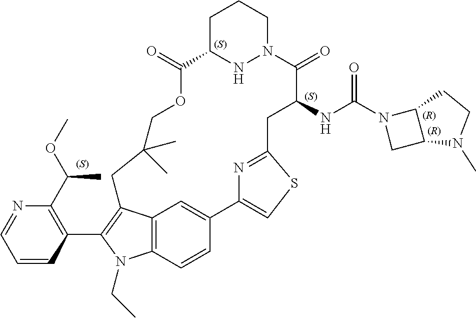 |
| A554 | 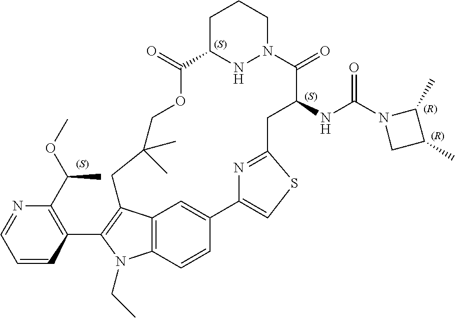 |
| A555 | 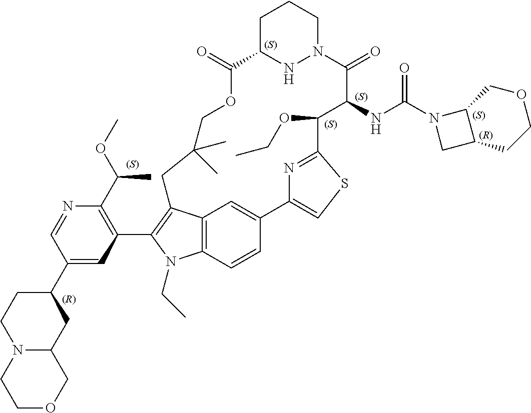 |
| A556 | 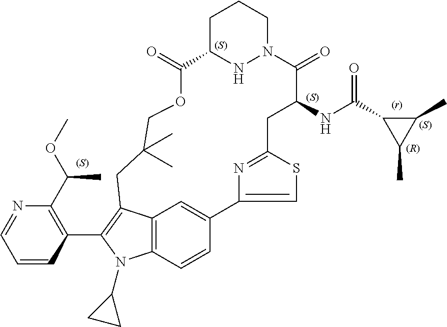 |
| A557 | 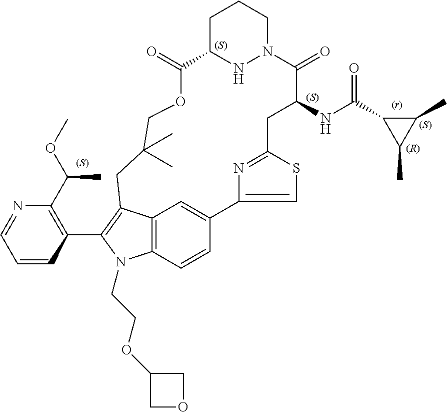 |
| A558 | 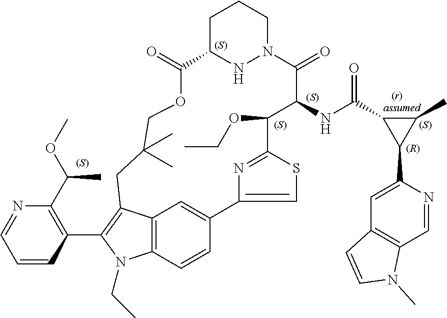 |
| A559 | 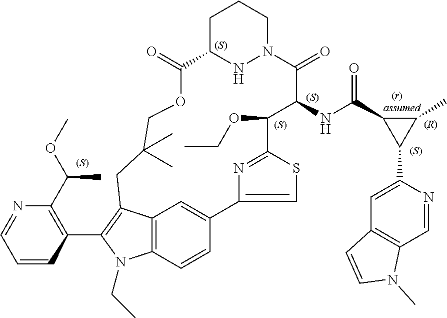 |
| A560 | 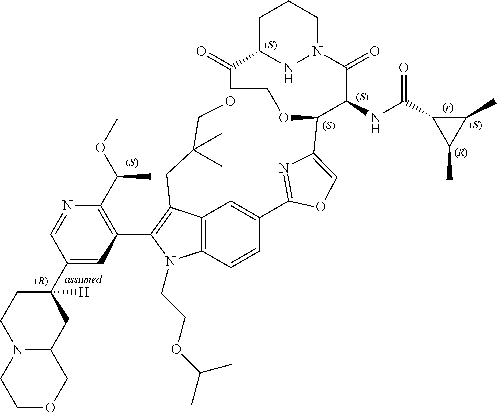 |
| A561 | 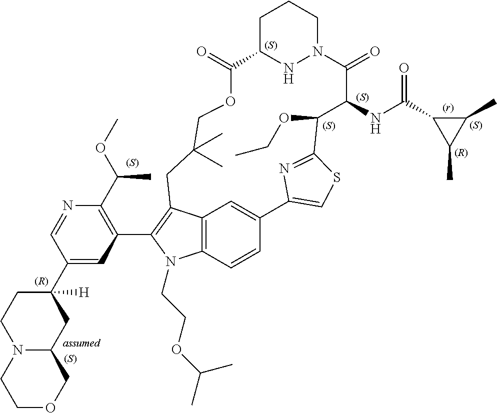 |
| A562 | 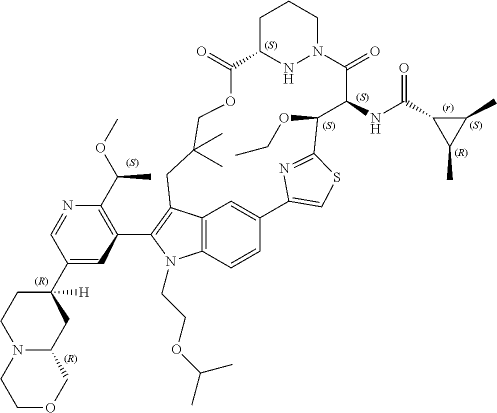 |
| A563 | 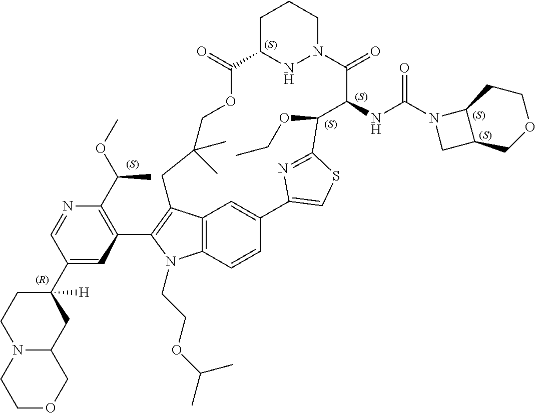 |
| A564 | 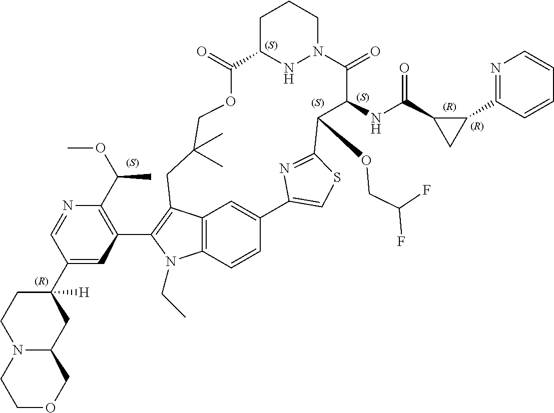 |
| A565 | 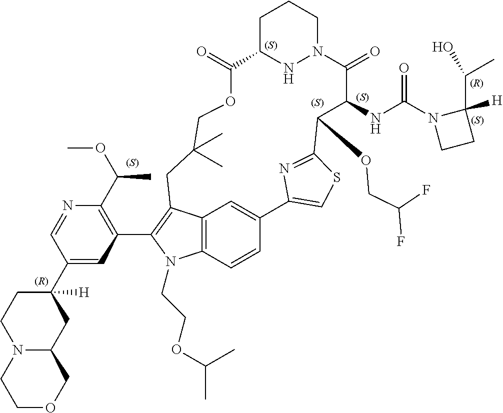 |
| A566 | 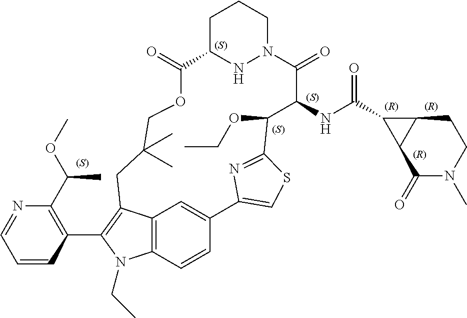 |
| A567 | 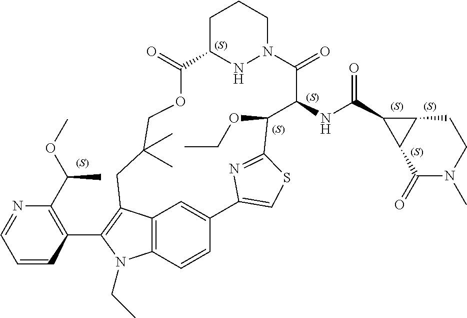 |
| A568 | 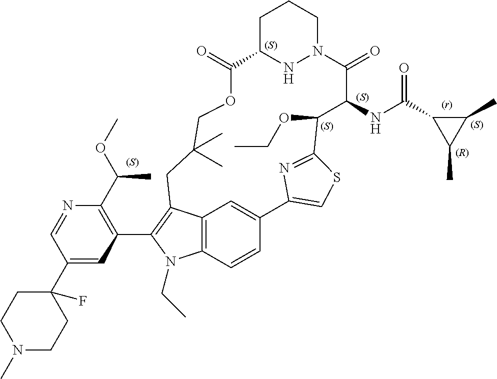 |
| A569 | 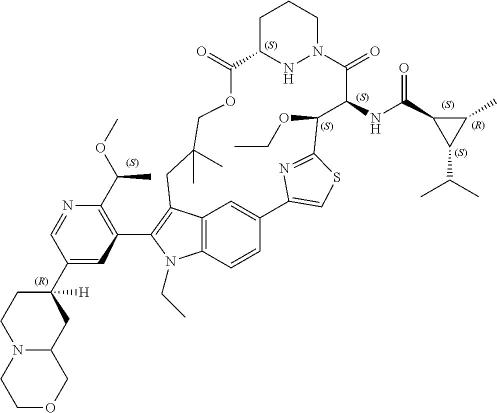 |
| A570 | 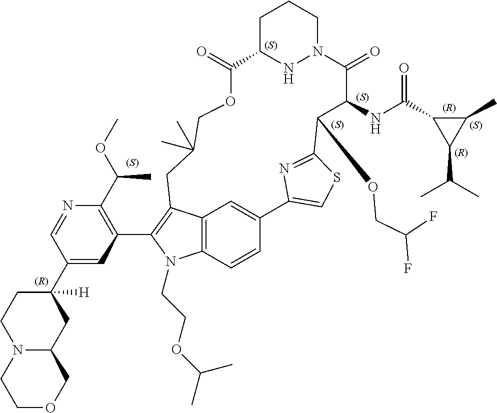 |
| A571 | 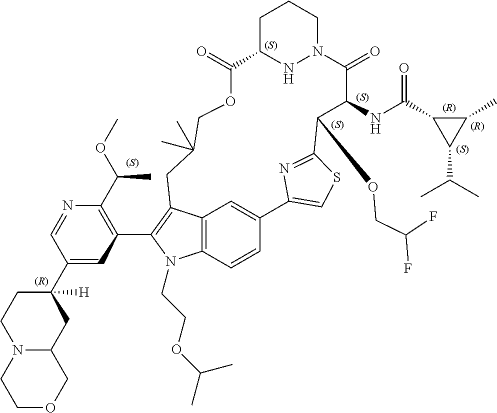 |
| A572 | 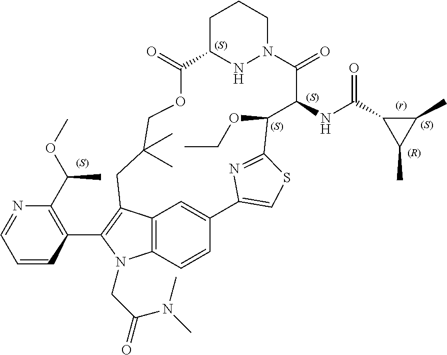 |
| A573 | 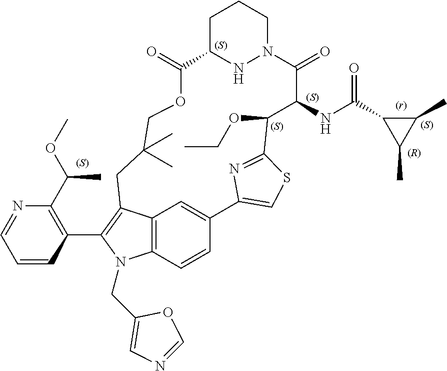 |
| A574 | 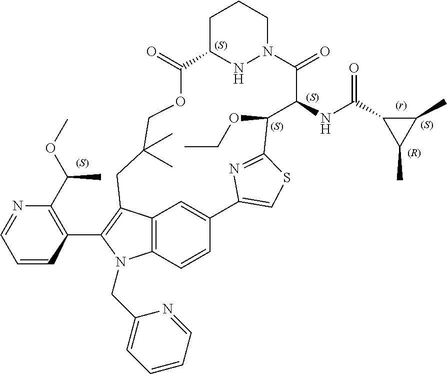 |
| A575 | 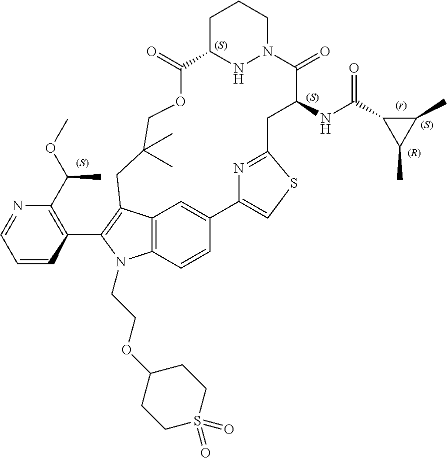 |
| A576 | 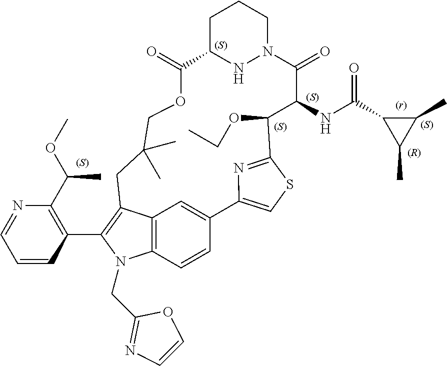 |
| A577 | 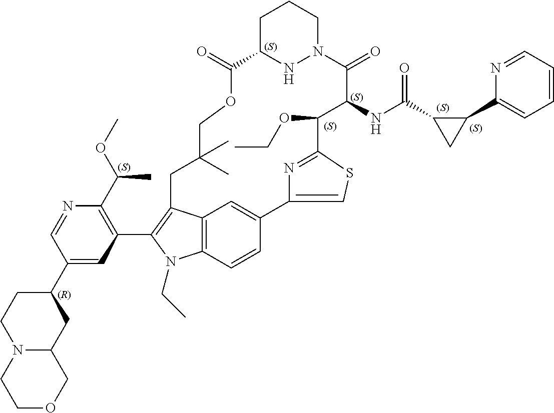 |
| A578 | 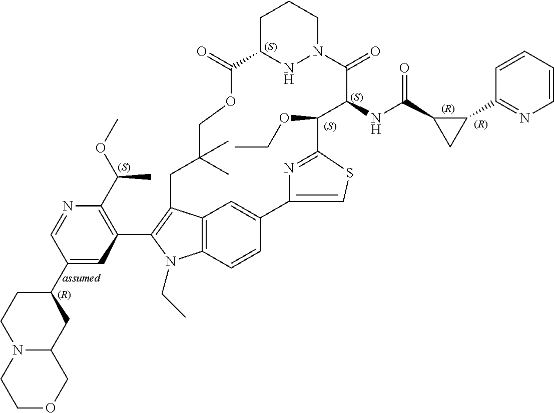 |
| A579 | 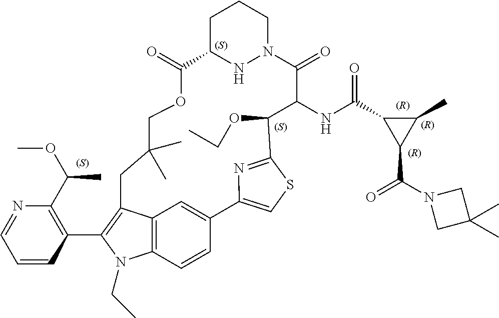 |
| A580 | 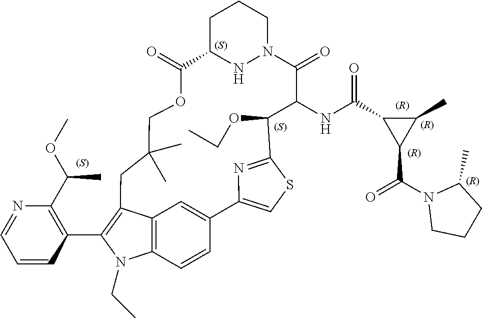 |
| A581 | 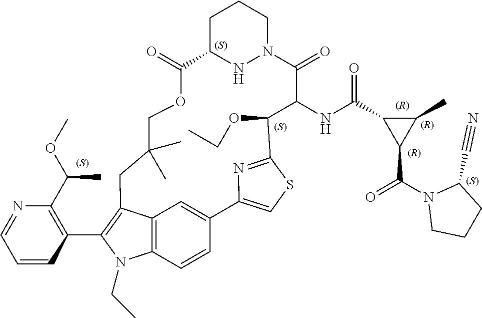 |
| A582 | 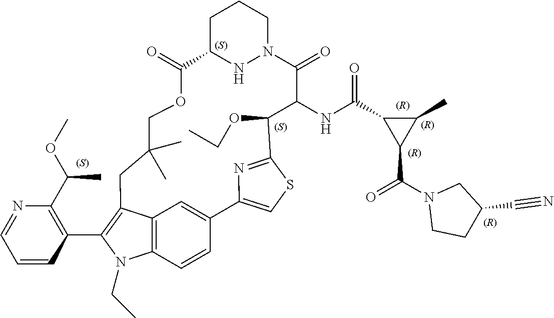 |
| A583 | 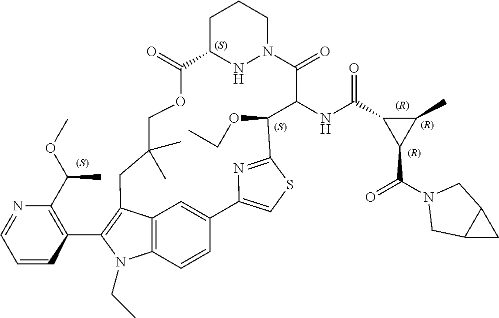 |
| A584 | 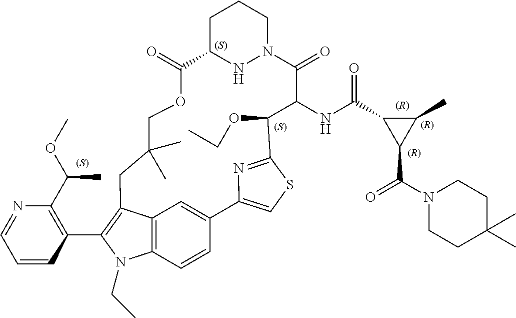 |
| A585 | 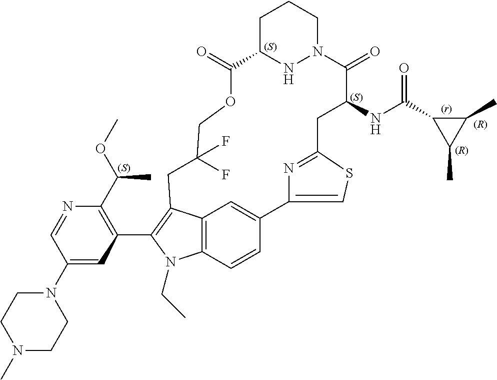 |
| A586 | 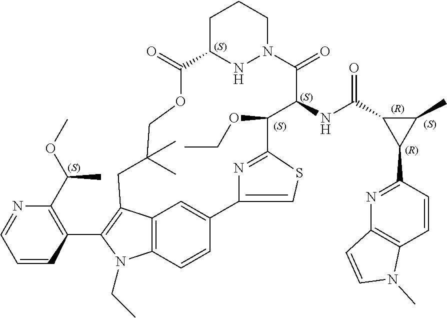 |
| A587 | 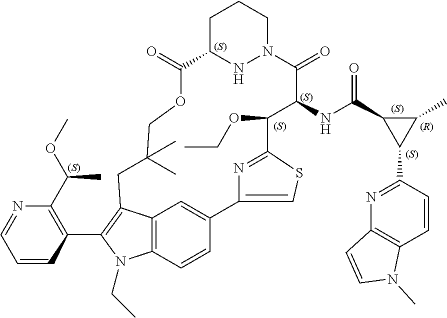 |
| A588 | 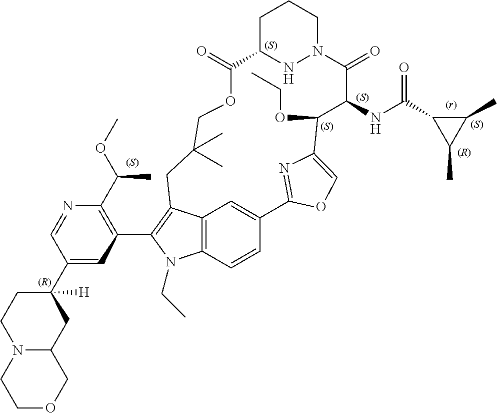 |
| A589 | 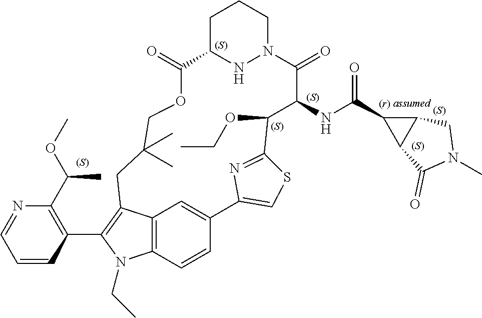 |
| A590 | 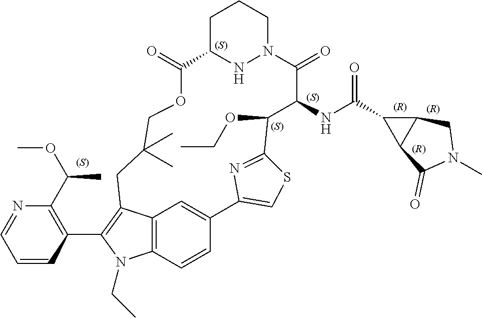 |
| A591 | 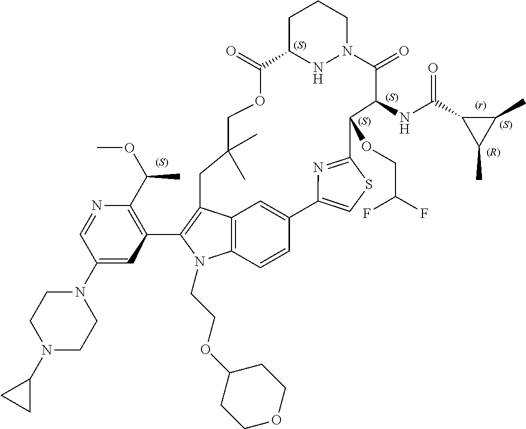 |
| A592 | 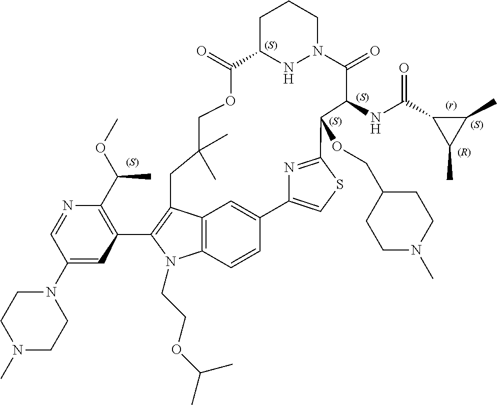 |
| A593 | 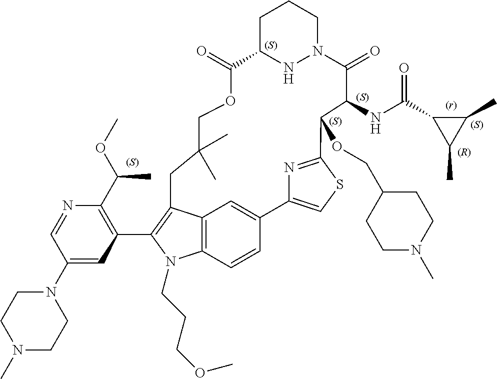 |
| A594 | 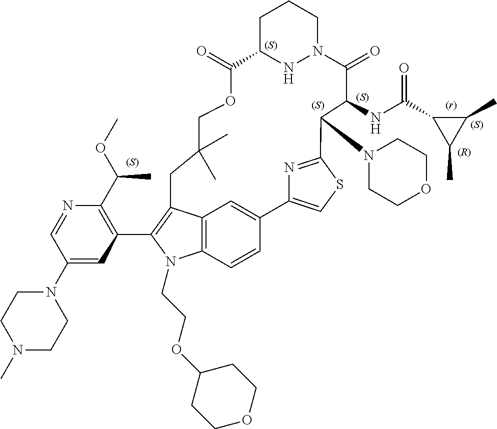 |
| A595 | 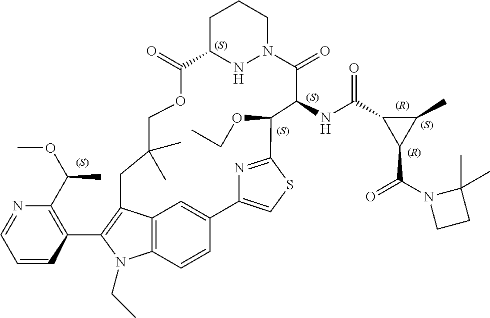 |
| A596 | 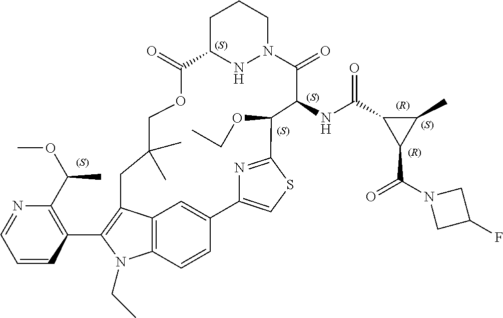 |
| A597 | 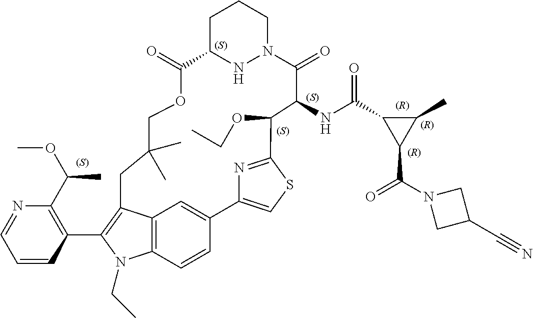 |
| A598 | 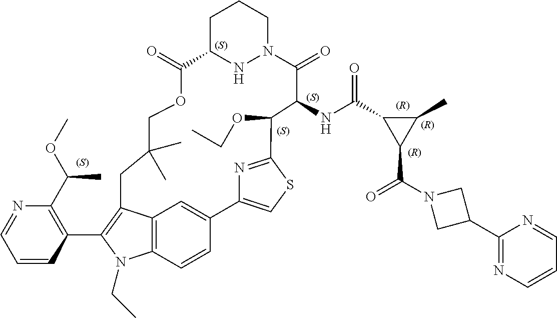 |
| A599 | 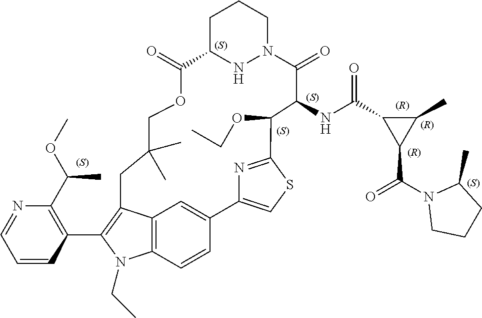 |
| A600 | 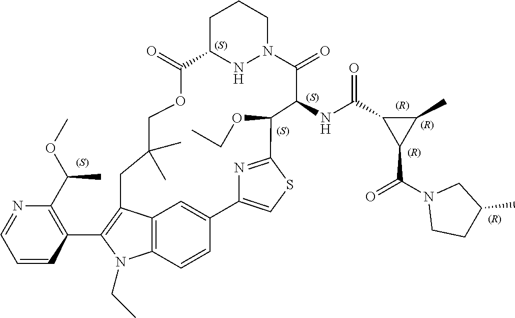 |
| A601 | 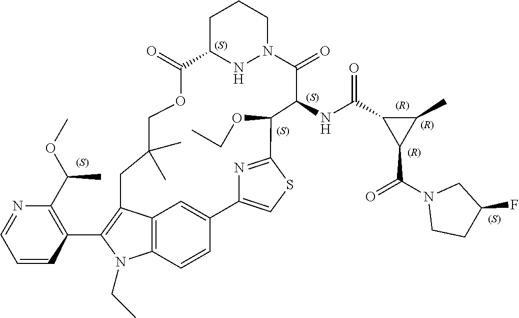 |
| A602 | 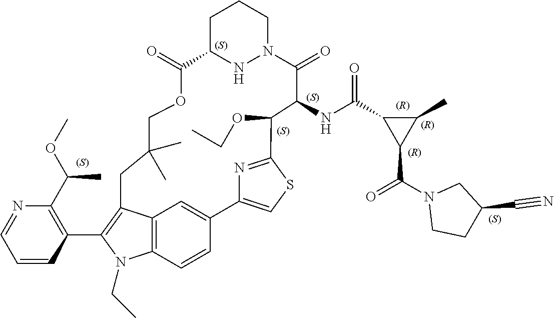 |
| A603 | 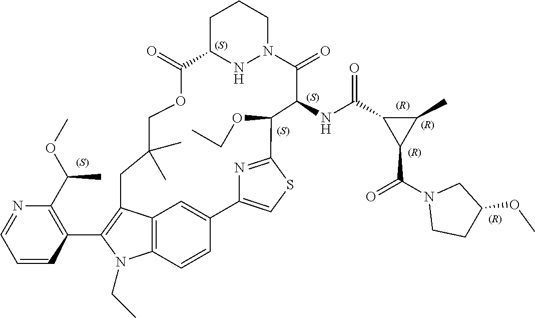 |
| A604 | 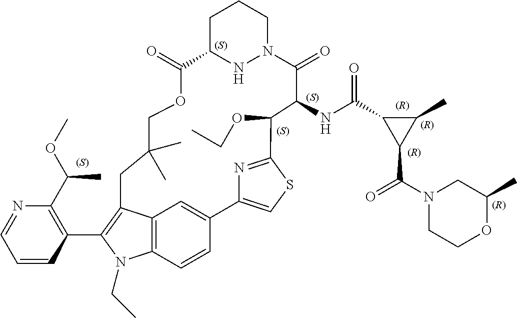 |
| A605 | 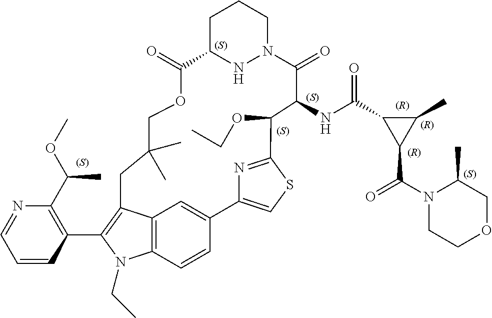 |
| A606 | 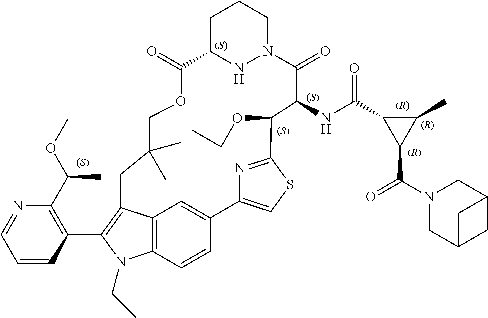 |
| A607 | 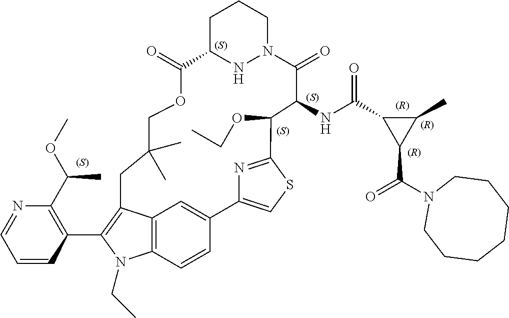 |
| A608 | 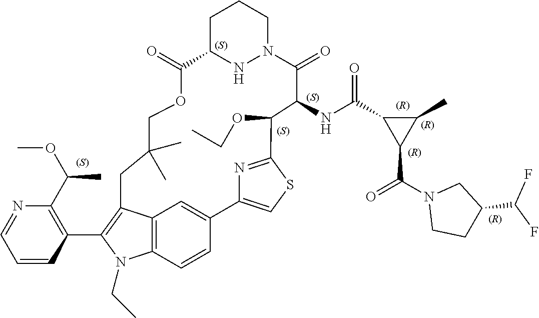 |

In some embodiments, a compound of the present invention is a compound selected from Table 2, or a pharmaceutically acceptable salt or stereoisomer thereof. In some embodiments, a compound of the present invention is a compound selected from Table 2, or a pharmaceutically acceptable salt or atropisomer thereof

In some embodiments, a compound of the present invention is not a compound selected from Table 2. In some embodiments, a compound of the present invention is not a compound selected from Table 2, or a pharmaceutically acceptable salt or stereoisomer thereof. In some embodiments, a compound of the present invention is not a compound selected from Table 2, or a pharmaceutically acceptable salt or atropisomer thereof.

TABLE 2
Certain Compounds

| Ex. # | Structure |
|---|---|
| B1 | 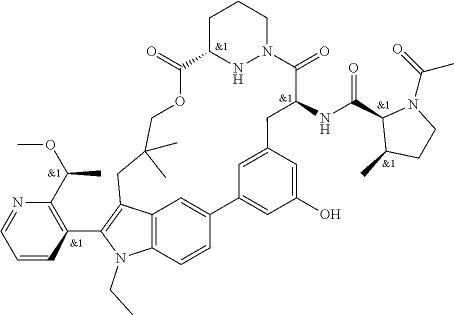 |
| B2 | 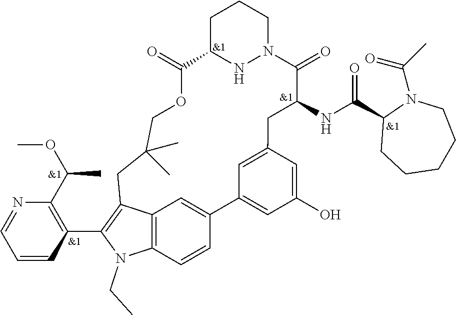 |
| B3 | 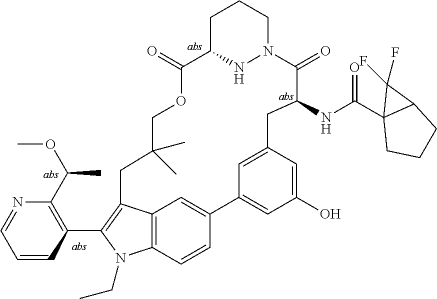 |
| B4 | 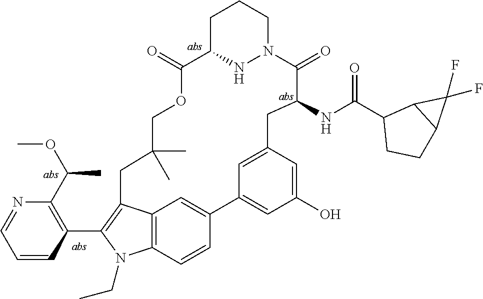 |
| B5 | 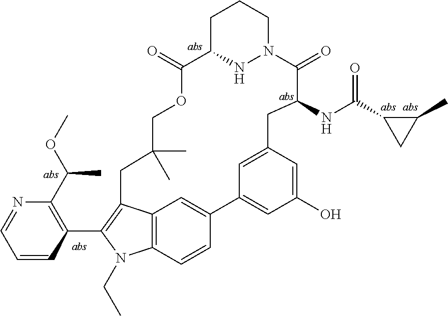 |
| B6 | 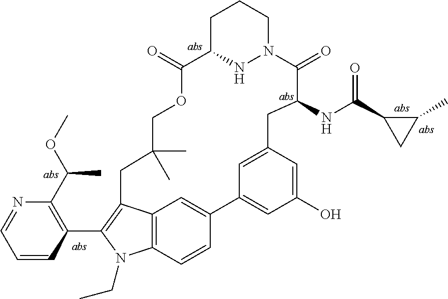 |
| B7 | 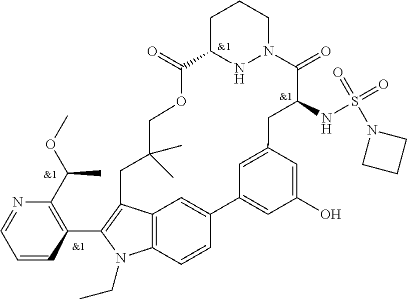 |
| B8 | 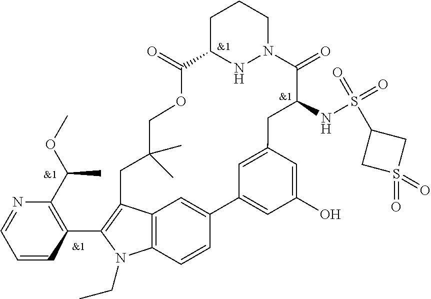 |
| B9 | 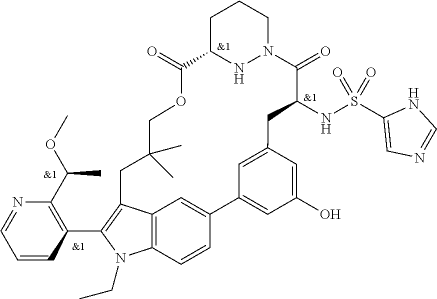 |
| B10 | 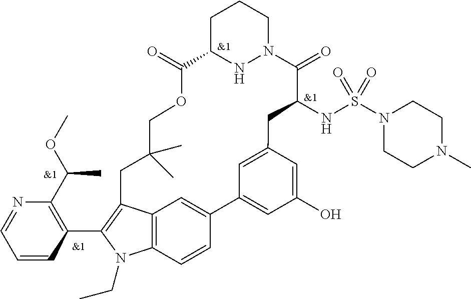 |
| B11 | 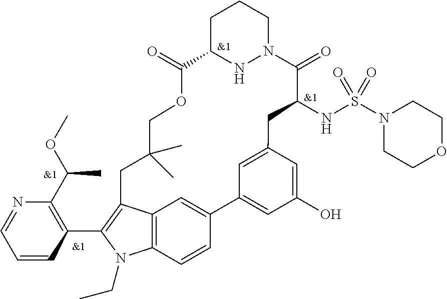 |
| B12 | 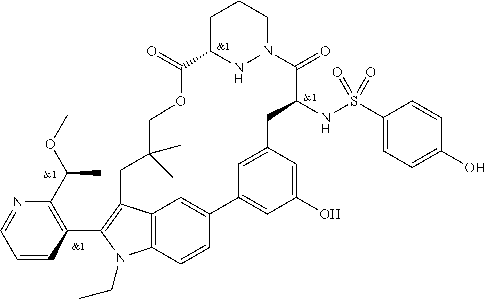 |
| B13 | 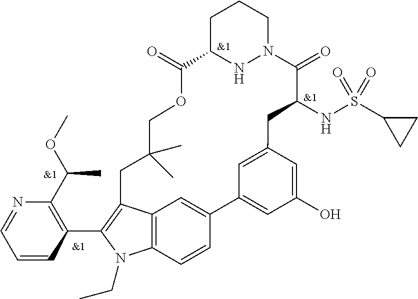 |
| B14 | 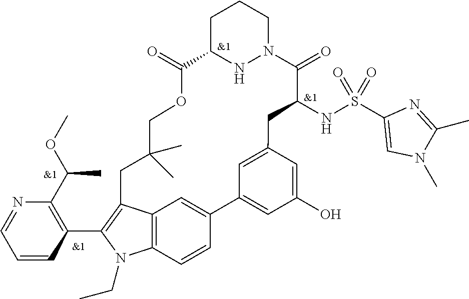 |
| B15 | 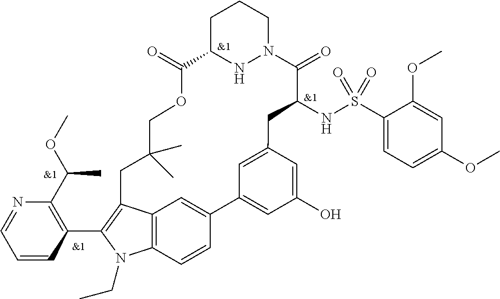 |
| B16 | 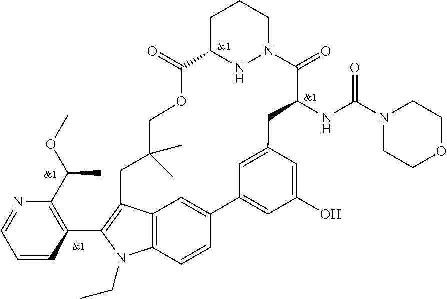 |
| B17 | 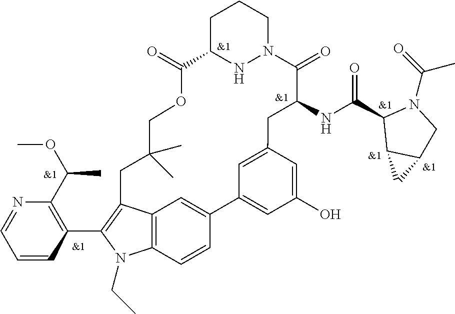 |
| B18 | 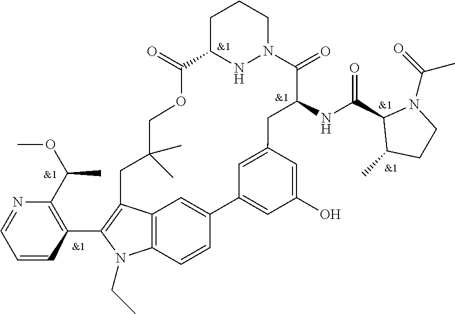 |
| B19 | 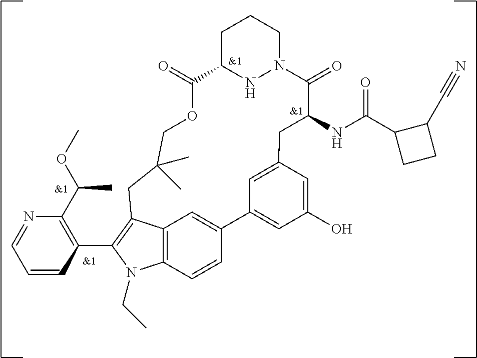 |
| B20 | 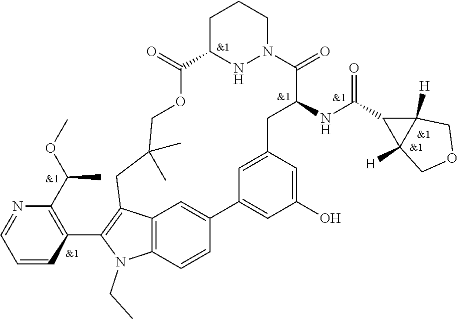 |
| B21 | 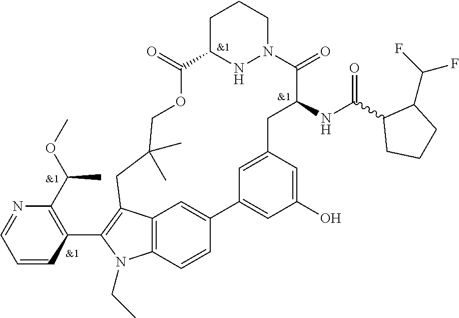 |
| B22 | 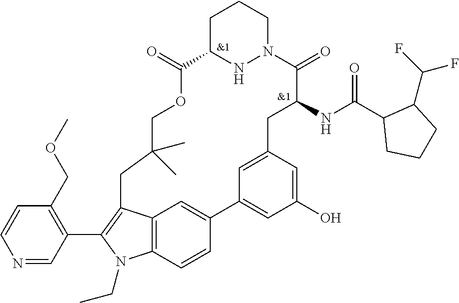 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| B23 | 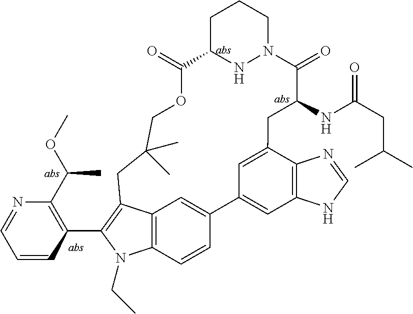 |
| B24 | 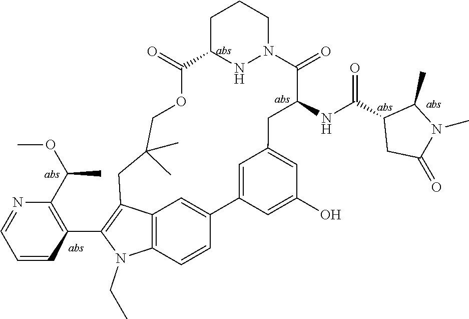 |
| B25 | 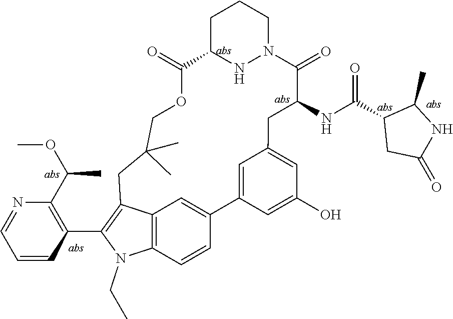 |

In some embodiments, a compound of the present invention is a compound selected from Table 3 (e.g., C1-C20 or C1-C21), or a pharmaceutically acceptable salt or stereoisomer thereof. In some embodiments, a compound of the present invention is a compound selected from Table 3 5 (e.g., C1-C20 or C1-C21), or a pharmaceutically acceptable salt or atropisomer thereof.

In some embodiments, a compound of the present invention is not a compound selected from Table 3 (e.g., C1-C20 or C1-C21). In some embodiments, a compound of the present invention is not a compound selected from Table 3 (e.g., C1-C20 or C1-C21), or a pharmaceutically acceptable salt or stereoisomer thereof. In some embodiments, a compound of the present invention is not a compound selected from Table 3 (e.g., C1-C20 or C1-C21), or a pharmaceutically acceptable salt or atropisomer thereof.

TABLE 3
Certain Compounds

| Ex. # | Structure |
|---|---|
| C1 | 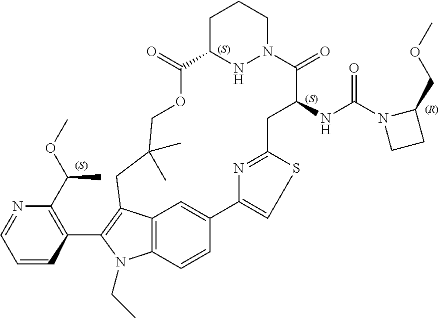 |
| C2 | 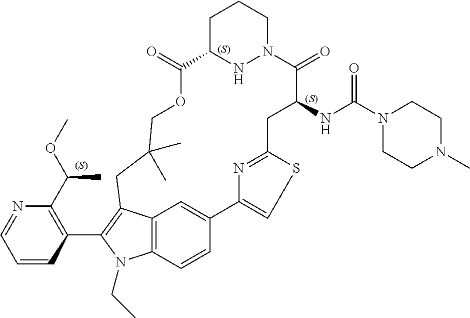 |
| C3 | 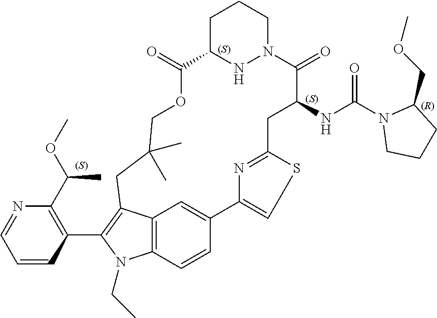 |
| C4 | 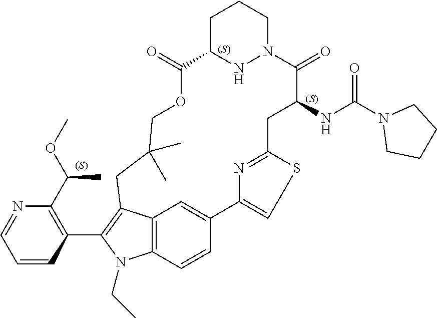 |
| C5 | 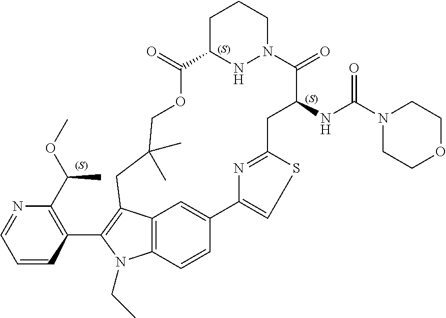 |
| C6 | 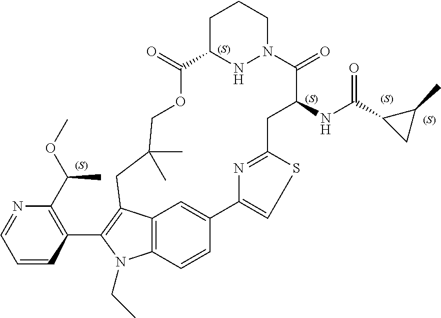 |
| C7 | 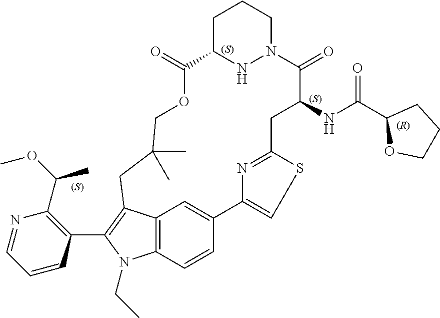 |
| C8 | 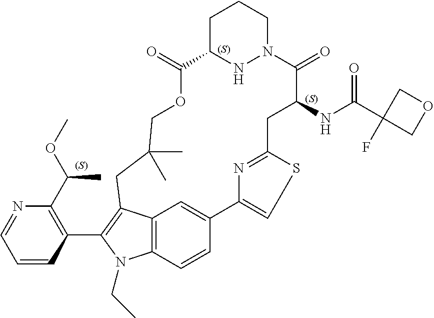 |
| C9 | 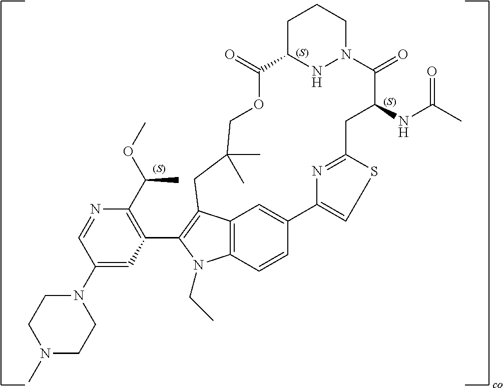 |
| C10 | 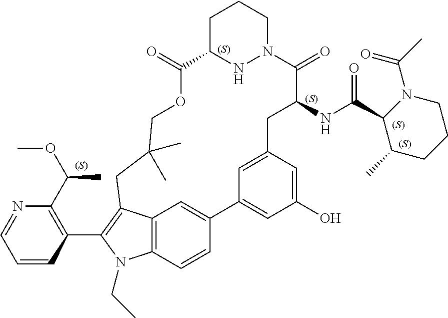 |
| C11 | 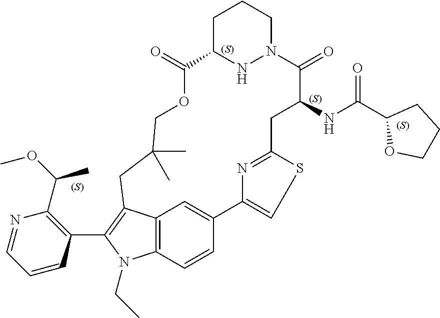 |
| C12 | 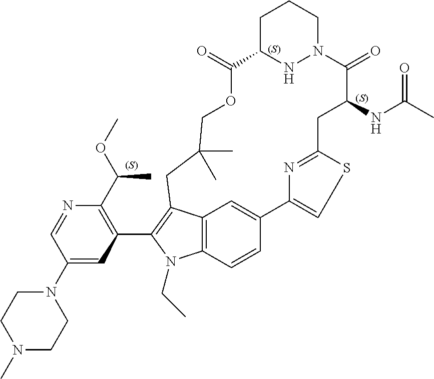 |
| C13 | 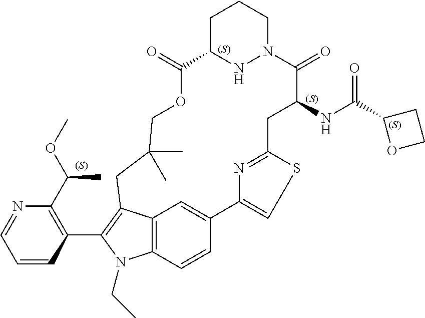 |
| C14 | 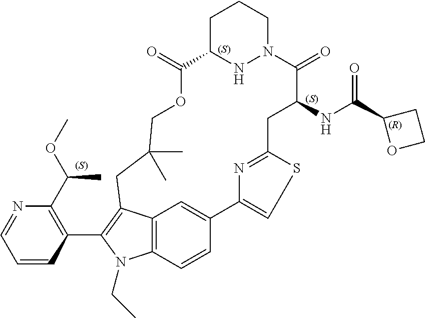 |
| C15 | 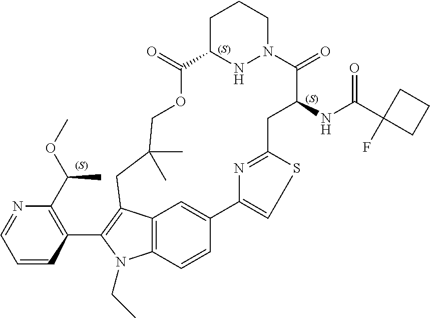 |
| C16 | 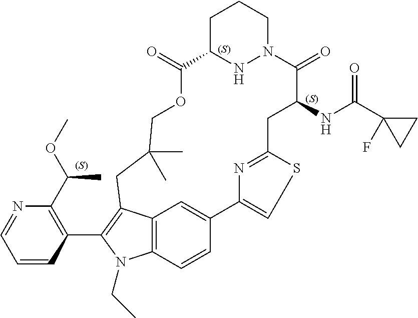 |
| C17 | 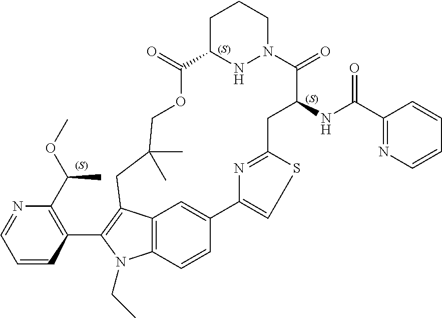 |
| C18 | 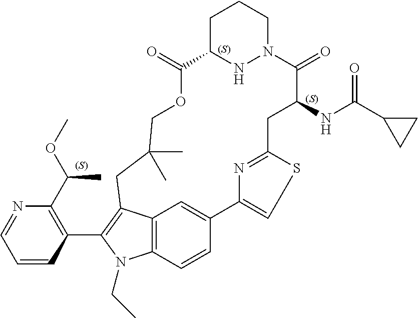 |
| C19 | 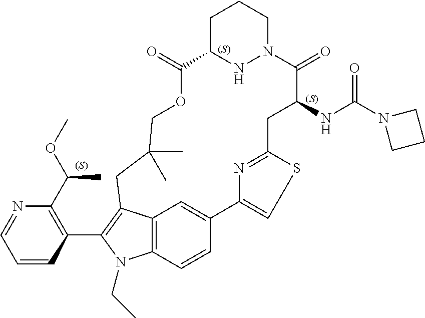 |
| C20 | 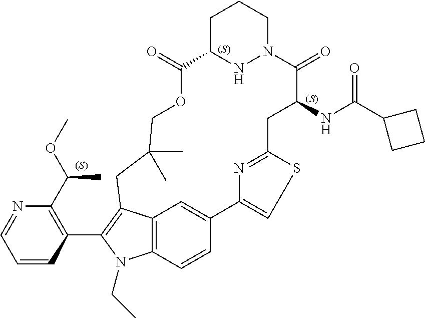 |
| C21 | 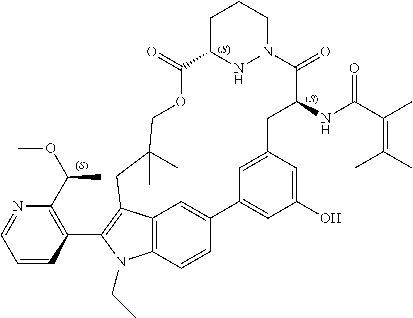 |

In some embodiments, a compound of the present invention has improved oral bioavailability compared to what is known in the art. Methods of measuring oral bioavailability are known in the art, and one such method is provided in the Examples below.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

With respect to compounds of the present invention, one stereoisomer may exhibit better inhibition than another stereoisomer. For example, one atropisomer may exhibit inhibition, whereas the other atropisomer may exhibit little or no inhibition.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

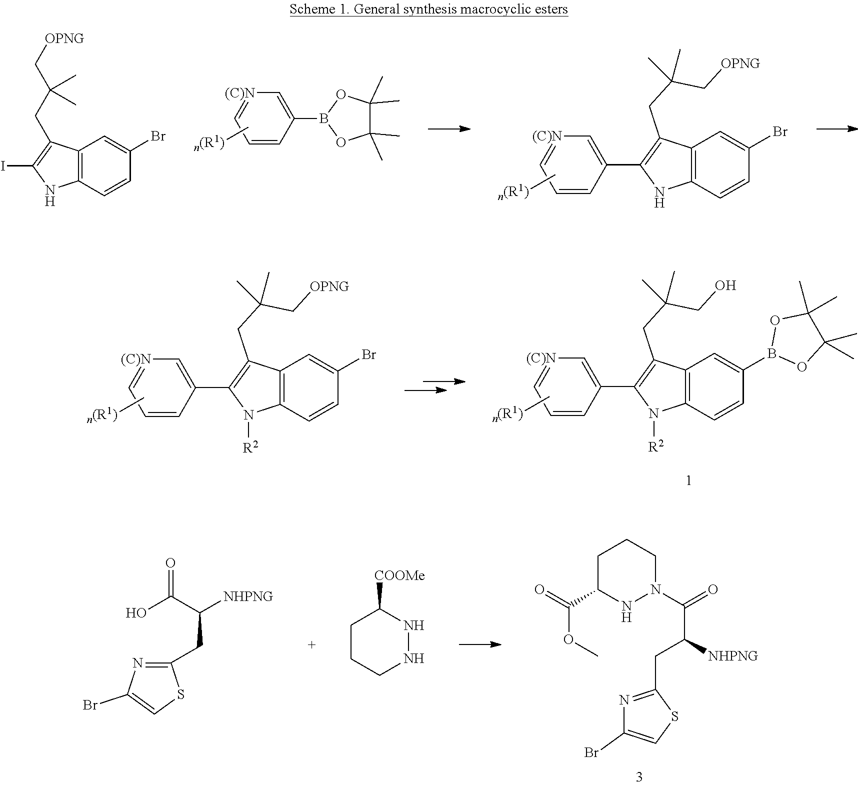

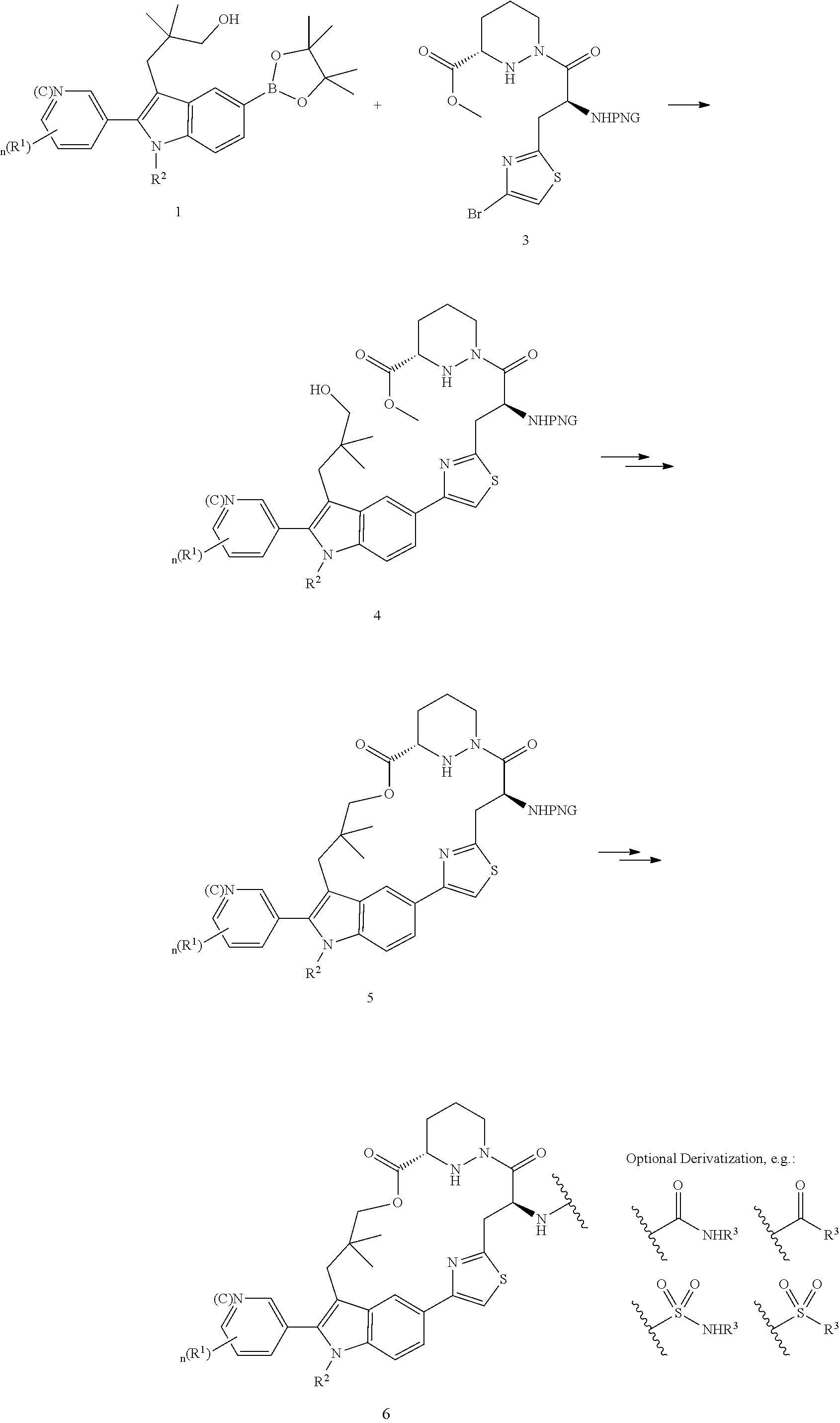

A general synthesis of macrocyclic esters is outlined in Scheme 1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl) propanoyl) hexahydropyridazine-3-carboxylate (3) can be prepared via coupling of(S)-2-amino-3-(4-bromothiazol-2-yl) propanoic acid (2) with methyl(S)-hexahydropyridazine-3-carboxylate.

The final macrocyclic esters can be made by coupling of methyl-amino-3-(4-bromothiazol-2-yl) propanoyl) hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme 1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

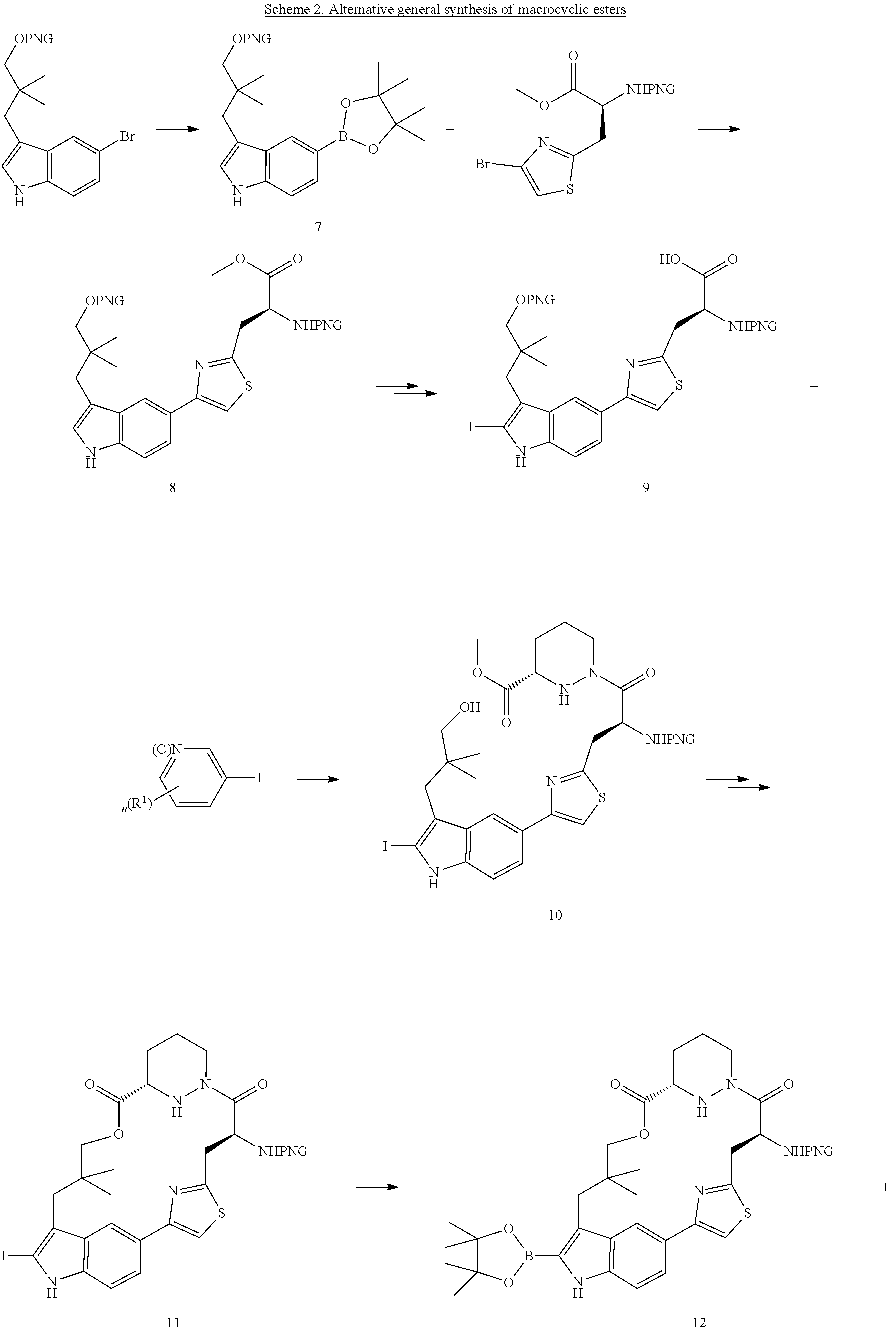

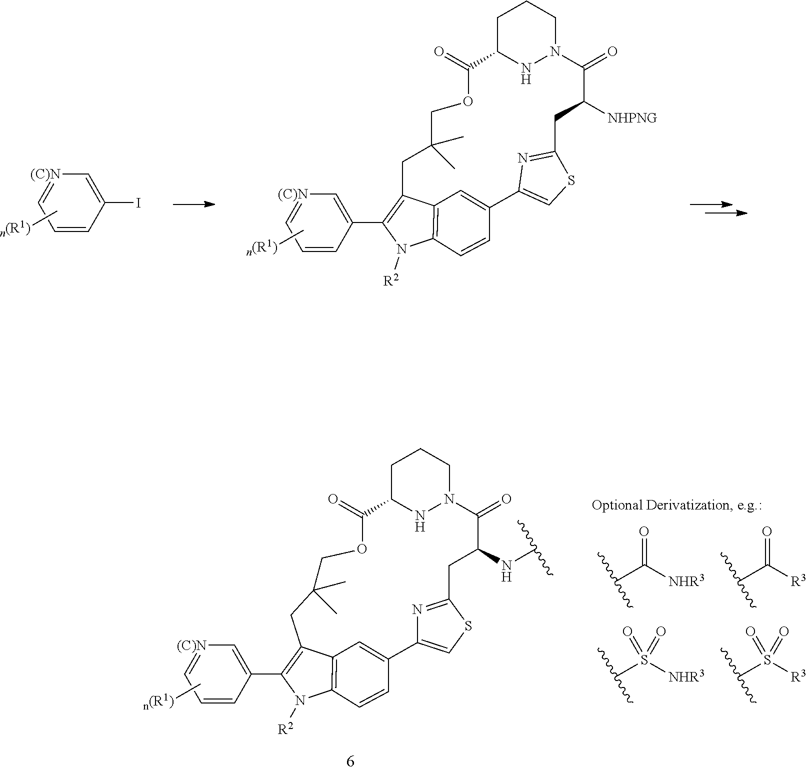

Alternatively, macrocyclic esters can be prepared as described in Scheme 2. An appropriately substituted and protected indolyl boronic ester (7) can be coupled in the presence of Pd catalyst with(S)-2-amino-3-(4-bromothiazol-2-yl) propanoic acid, followed by iodination, deprotection, and ester hydrolysis. Subsequent coupling with methyl(S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme 2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table 1 and Table 1-1 herein were prepared using methods disclosed herein or were prepared using methods described herein combined with the knowledge of one of skill in the art.

### Pharmaceutical Compositions and Methods of Use

### Pharmaceutical Compositions and Methods of Administration

The compounds with which the invention is concerned are Ras inhibitors, and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc.

Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:

| | |
|---|---|
| B₂pin₂ | Bis(pinacolato)diboron |
| BINAP | 2,2′-Bis(diphenylphosphino)-1,1-binaphthyl |
| CH₂Cl₂, DCM | Methylene chloride, Dichloromethane |
| CH₃CN, MeCN | Acetonitrile |
| CuI | Copper (I) iodide |
| DIPEA, DIEA | Diisopropylethyl amine |
| DMF | N,N-Dimethylformamide |
| EA | Ethyl acetate |
| EDCl | N-Ethyl-N′-carbodiimide hydrochloride |
| EtOAc | Ethyl acetate |
| h | hour |
| H₂O | Water |
| HCl | Hydrochloric acid |
| HOBt | Hydroxybenzotriazole |
| K₃PO₄ | Potassium phosphate (tribasic) |
| MeOH | Methanol |
| Na₂SO₄ | Sodium sulfate |
| NMM | N-methylmorpholine |
| NMP | N-methyl pyrrolidone |
| Pd(dppf)Cl₂ | [1,1′-Bis(diphenylphosphino)ferrocene] |
| | dichloropalladium(II) |
| PE | Petroleum ether |
| rt | Room temperature |
| TFA | Trifluoroacetic acid |

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

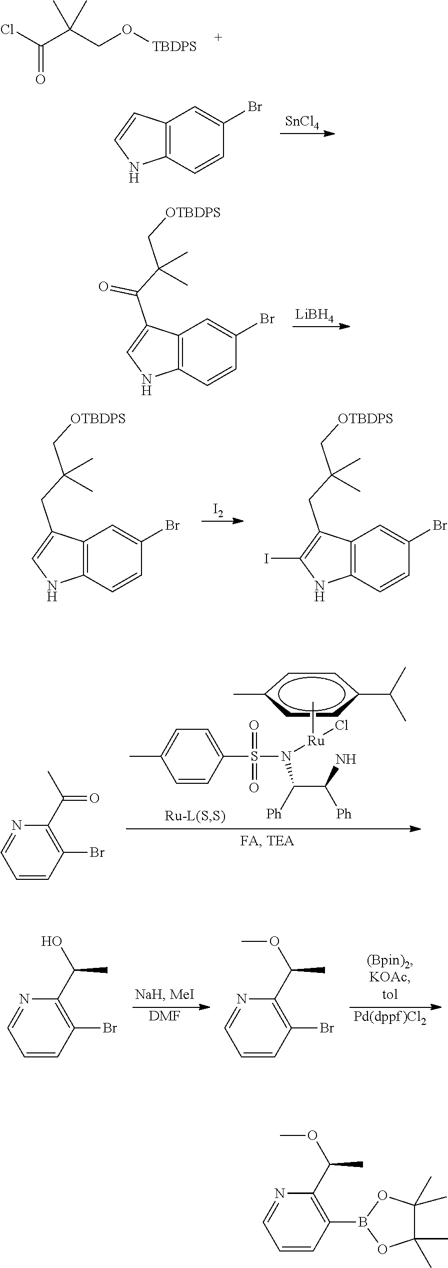

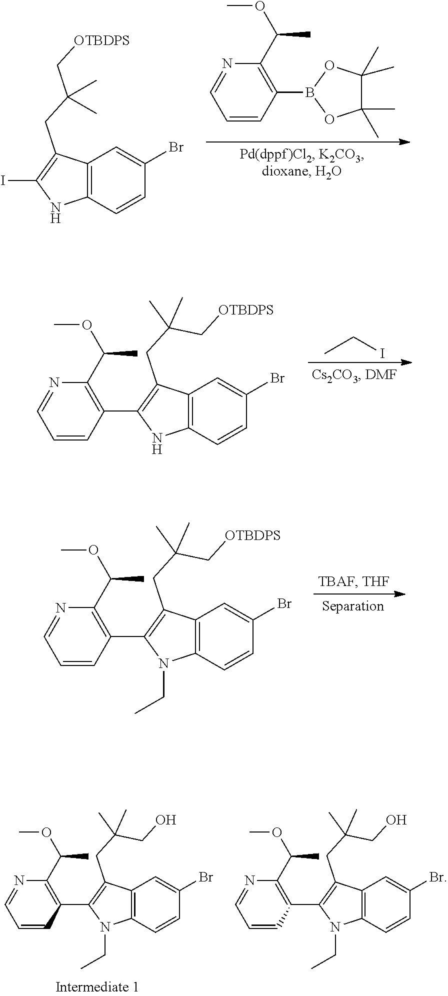

### Step 1

To a mixture of 3-((tert-butyldiphenylsilyl)oxy)-2,2-dimethylpropanoyl chloride (65 g, 137 mmol, crude) in DCM (120 mL) at 0° C. under an atmosphere of N2 was added 1M SnCl4 in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (400 mL), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na] calc'd for C29H32BrNO2SiNa 556.1; found 556.3.

### Step 2

To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N2 was added LiBH4 (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH. H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C29H34BrNOSi 519.2; found 520.1; ¹H NMR (400 MHZ, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

### Step 3

To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHZ, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

### Step 4

To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl) ethanol (100 g, 74% yield) as an oil. LCMS (ESI): m/z [M+H] calc'd for C₇H₈BrNO 201.1; found 201.9.

### Step 5

To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1 S)-1-methoxyethyl]pyridine (90 g, 75% yield) as an oil. LCMS (ESI): m/z [M+H] calc'd for C₈H₁₀BrNO 215.0; found 215.9.

### Step 6

To a stirred mixture of 3-bromo-2-[(1 S)-1-methoxyethyl]pyridine (90 g, 417 mmol) and Pd(dppf)Cl₂ (30.5 g, 41.7 mmol) in toluene (900 mL) at rt under an atmosphere of Ar was added bis (pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl) pyridine (100 g, 63% yield) as a semi-solid. LCMS (ESI): m/z [M+H] calc'd for C₁₄H₂₂BNO₃ 263.2; found 264.1.

### Step 7

To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl) pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1 S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.

### Step 8

To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and EtI (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.

### Step 9

To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give two atropisomers of 3-(5-bromo-1-ethyl-2-(2-((S)-1-methoxyethyl) pyridin-3-yl)-1H-indol-3-yl)-2,2-dimethylpropan-1-ol (as single atropisomers) both as solids. (Combined 30 g, 62% yield) both as a solid. LCMS (ESI): m/z [M+H] calc'd for C₂₃H₂₉BrN₂O₂ 444.1; found 445.1.

### Intermediate 1. Alternative Synthesis through Fisher Indole Route

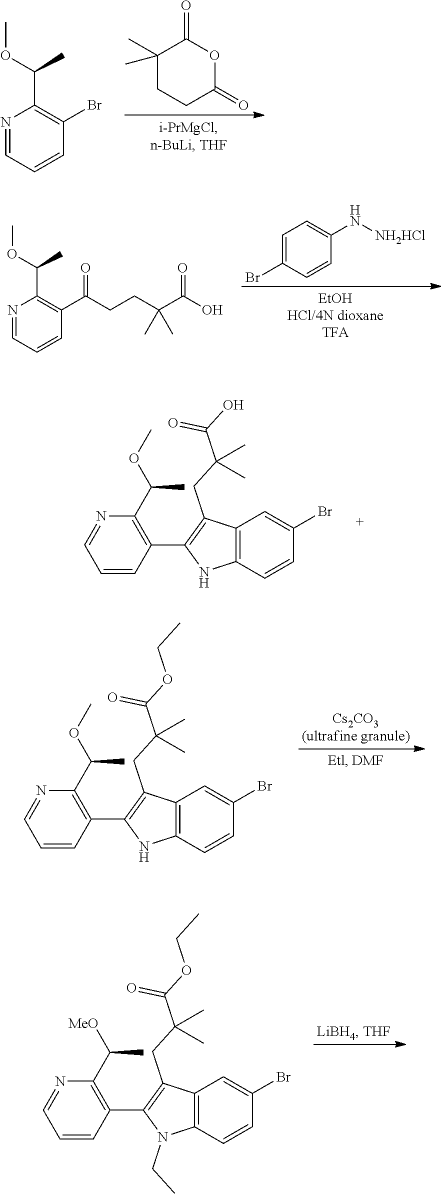

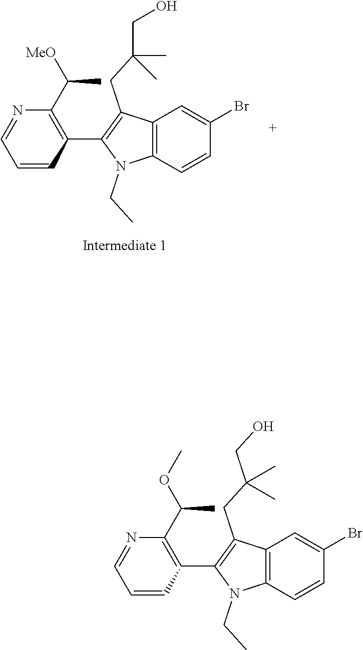

### Step 1

To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at-10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.

### Step 2

To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl) hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5, concentrated under reduced pressure and the residue adjusted to PH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

### Step 3

To a mixture of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl) pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. EtI (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1 S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.

### Step 4

To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl) pyridin-3-yl)-1H-indol-3-yl)-2,2-dimethylpropan-1-ol (as single atropisomers) (60 g, 38% yield) and (40 g, 26% yield) both as solids. LCMS (ESI): m/z [M+H] calc'd for C₂₃H₂₉BrN₂O₂ 444.1; found 445.2.

### Intermediate 2. Synthesis of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

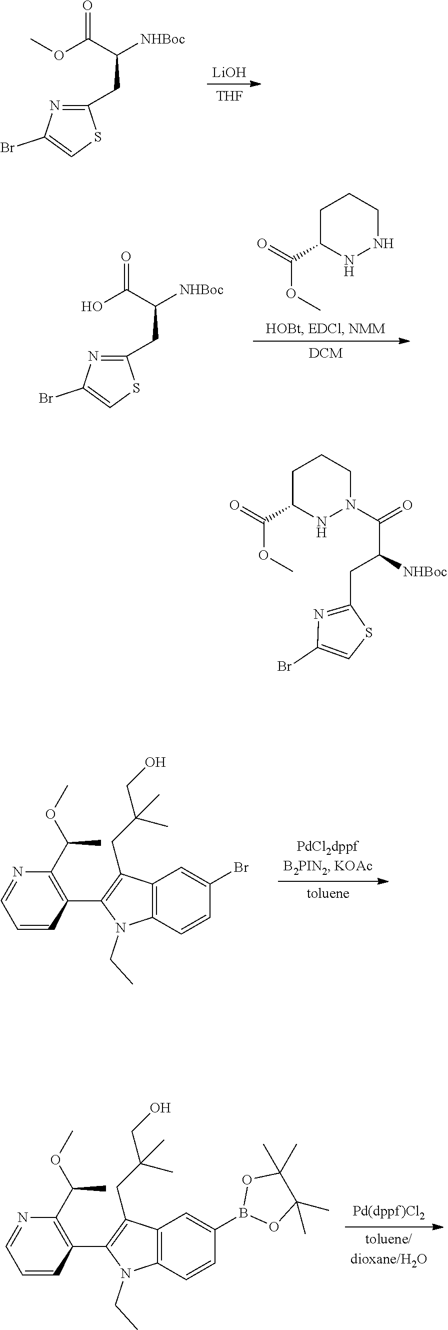

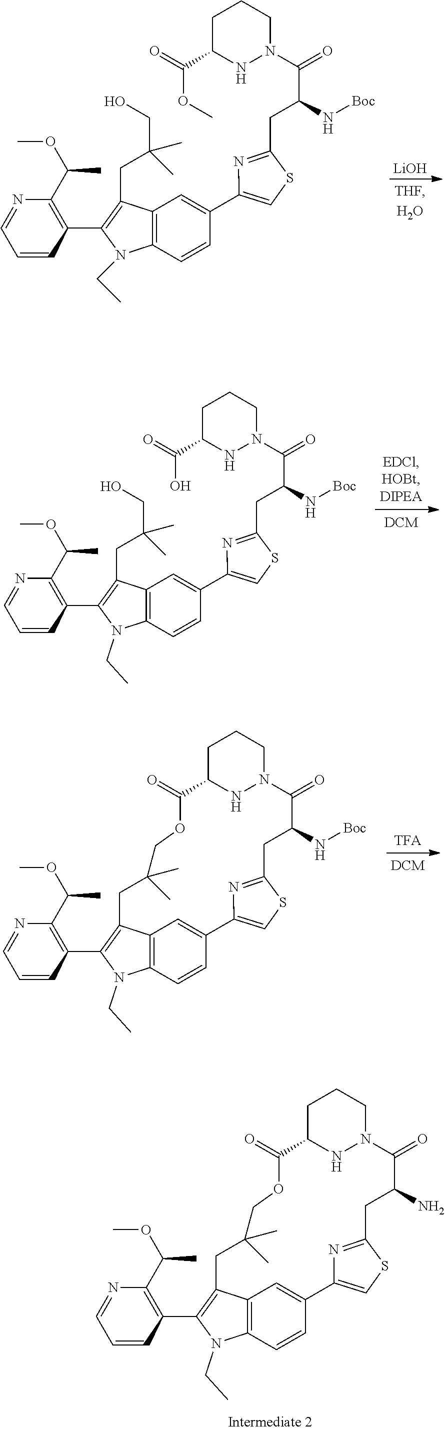

### Step 1

To a solution of methyl (2S)-3-(4-bromo-1,3-thiazol-2-yl)-2-[(tert-butoxycarbonyl) amino]propanoate (110 g, 301.2 mmol) in THF (500 mL) and H₂O (200 mL) at room temperature was added LiOH (21.64 g, 903.6 mmol). The solution was stirred for 1 h and was then concentrated under reduced pressure. The residue was adjusted to pH 6 with 1 M HCl and then extracted with DCM (3×500 mL). The combined organic layers were, dried over Na₂SO₄, filtered, and concentrated under reduced pressure to give (S)-3-(4-bromothiazol-2-yl)-2-((tert-butoxycarbonyl) amino) propanoic acid (108 g, crude). LCMS (ESI): m/z [M+H] calc'd for C₁₁H₁₆BrN₂O₄S 351.0; found 351.0.

### Step 2

To a solution of(S)-3-(4-bromothiazol-2-yl)-2-((tert-butoxycarbonyl) amino) propanoic acid (70 g, 199.3 mmol) in DCM (500 mL) at 0° C. was added methyl (3S)-1,2-diazinane-3-carboxylate bis (trifluoroacetic acid) salt (111.28 g, 298.96 mmol), NMM (219.12 mL. 1993.0 mmol), EDCI (76.41 g, 398.6 mmol) and HOBt (5.39 g, 39.89 mmol). The solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel column chromatography to give methyl(S)-1-((S)-3-(4-bromothiazol-2-yl)-2-((tert-butoxycarbonyl) amino) propanoyl) hexahydropyridazine-3-carboxylate (88.1 g, 93% yield). LCMS (ESI): m/z [M+H] calc'd for C₁₇H₂₆BrN₄O₅S 477.1; found 477.1.

### Step 3

To a solution of 3-(5-bromo-1-ethyl-2-(2-((S)-1-methoxyethyl) pyridin-3-yl)-1H-indol-3-yl)-2,2-dimethylpropan-1-ol (60 g, 134.7 mmol) in toluene (500 mL) at room temperature was added bis (pinacolato)diboron (51.31 g, 202.1 mmol), Pd(dppf)Cl₂ (9.86 g, 13.4 mmol), and KOAc (26.44 g, 269 mmol). The reaction mixture was then heated to 90° C. and stirred for 2 h. The reaction solution was then cooled to room temperature and concentrated under reduced pressure. The residue was purified by silica gel column chromatography to give (S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (60.6 g, 94% yield). LCMS (ESI): m/z [M+H] calc'd for C₂₉H₄₂BN₂O₄ 493.32; found 493.3.

### Step 4

To a solution of(S)-3-(1-ethyl-2-(2-(1-methoxyethyl) pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl(S)-1-((S)-3-(4-bromothiazol-2-yl)-2-((tert-butoxycarbonyl) amino) propanoyl) hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The mixture was extracted with EtOAc and the combined organic layers were dried over Na₂SO₄, filtered, and concentrated under reduced pressure. The residue was purified by silica gel column chromatography to give methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate (39.7 g, 85% yield). LCMS (ESI): m/z [M+H] calc'd for C₄₀H₅₅N₆O₇S 763.4; found 763.3.

### Step 5

To a solution of methyl(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylate (39.7 g, 52.0 mmol) in THF (400 mL) and H₂O (100 mL) at room temperature was added LiOH·H₂O (3.74 g, 156.2 mmol). The mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to give (S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid (37.9 g, crude). LCMS (ESI): m/z [M+H] calc'd for C₃₉H₅₃N₆O₇S 749.4; found 749.4.

### Step 6

To a solution of(S)-1-((S)-2-((tert-butoxycarbonyl) amino)-3-(4-(1-ethyl-3-(3-hydroxy-2,2-dimethylpropyl)-2-(2-((S)-1-methoxyethyl) pyridin-3-yl)-1H-indol-5-yl) thiazol-2-yl) propanoyl) hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel column chromatography to give tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl) carbamate (30 g, 81% yield). LCMS (ESI): m/z [M+H] calc'd for C₃₉H₅₁N₆O₆S 731.4; found 731.3.

### Step 7

To a solution of tert-butyl ((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl) carbamate (6 g, 8.21 mmol) in DCM (60 mL) at 0° C. was added TFA (30 mL). The mixture was stirred for 1 h and was then concentrated under reduced pressure to give (63 S,4S,Z)-4-amino-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (7.0 g, crude). LCMS (ESI): m/z [M+H] calc'd for C₃₄H₄₂N₆O₄S 631.3; found: 630.3.

### Intermediate 3. Synthesis of tert-butyl ((6³S,4S,Z)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate

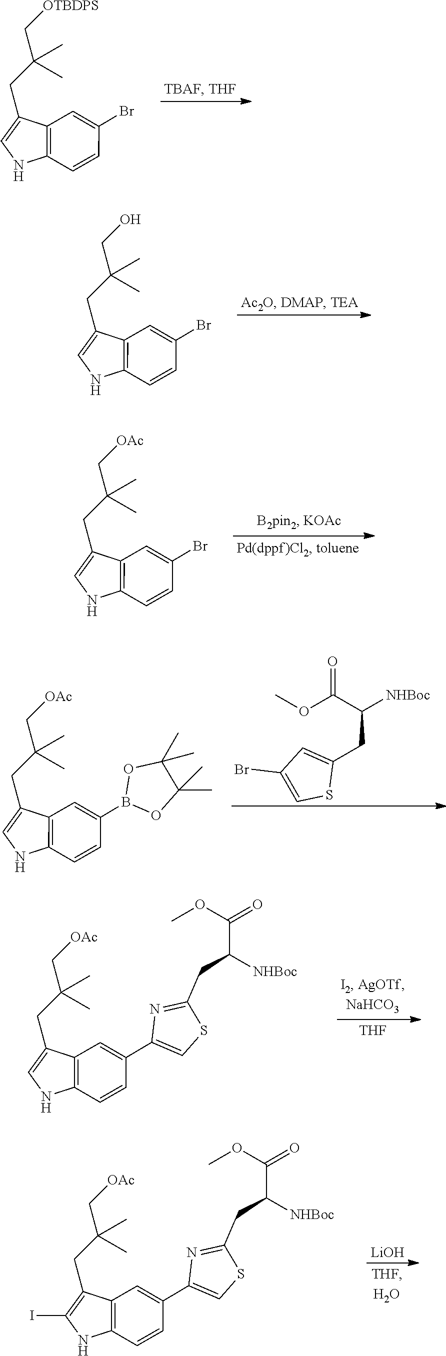

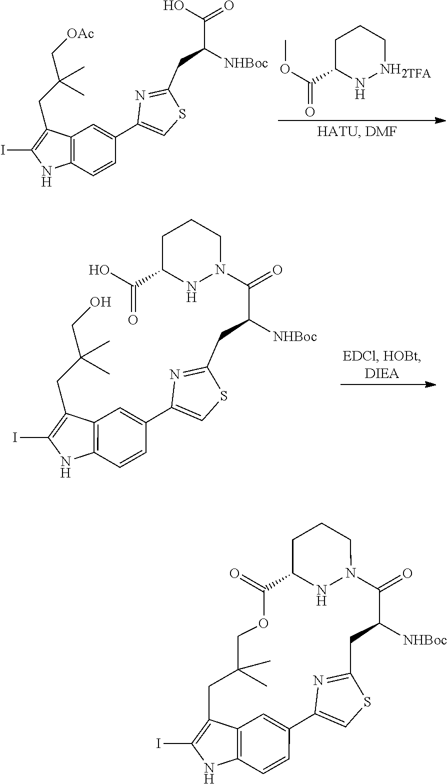

### Step 1

To a stirred solution of 5-bromo-3-[3-[(tert-butyldiphenylsilyl)oxy]-2,2-dimethylpropyl]-1H-indole (100 g, 192.0 mmol) in THF (1000 mL) were added TBAF (261.17 g, 998.8 mmol) in portion at room temperature. The resulting mixture was stirred for 16 h at 50° C. The resulting mixture was concentrated under reduced pressure. The resulting mixture was extracted with EtOAc (2 L). The combined organic layers were washed with water (6 L), dried over anhydrous Na₂SO₄. After filtration, the filtrate was concentrated under reduced pressure. The residue was purified by silica gel column chromatography, eluted with PE/EtOAc (3:1) to afford 3-(5-bromo-1H-indol-3-yl)-2,2-dimethylpropan-1-ol (54 g, 96.63%). LCMS (ESI): m/z [M+H] calc'd for C₁₃H₁₆BrNO 281.0; found 282.0.

### Step 2

To a stirred solution of 3-(5-bromo-1H-indol-3-yl)-2,2-dimethylpropan-1-ol (54 g, 191.3 mmol) in DCM (300 mL) were added TEA (58.09 g, 574.1 mmol) and Ac2O (18.95 g, 185.6 mmol) and DMAP (1.17 g, 9.5 mmol) dropwise at 0° C. The resulting mixture was washed with water (3×500 mL), dried over anhydrous Na₂SO₄. After filtration, the filtrate was concentrated under reduced pressure. This resulted in 3-(5-bromo-1H-indol-3-yl)-2,2-dimethylpropyl acetate (54 g, 80.6%) as a yellow solid. The crude product was used in the next step directly without further purification. LCMS (ESI): m/z [M+H] calc'd for C₁₅H₁₈BrNO₂ 323.0; found 324.0.

### Step 3

To a stirred solution of 3-(5-bromo-1H-indol-3-yl)-2,2-dimethylpropyl acetate (54 g, 166.5 mmol) in toluene (600 mL) were added KOAc (40.87 g, 416.3 mmol) and B₂pin₂ (105.76 g, 416.3 mmol) and Pd(dppf)Cl₂ (12.19 g, 16.6 mmol) in portions at room temperature under argon atmosphere. The resulting mixture was stirred for 3 h at 90° C. under argon atmosphere. The resulting mixture was concentrated under reduced pressure. The resulting mixture was extracted with EtOAc (1 L). The combined organic layers were washed with water (3×1 L), dried over anhydrous Na₂SO₄. After filtration, the filtrate was concentrated under reduced pressure. The residue was purified by silica gel column chromatography, eluted with PE/EtOAc (3:1) to afford 2,2-dimethyl-3-[5-(4,4,5,5-tetramethyl-1,3,2-dioxaborolan-2-yl)-1H-indol-3-yl]propyl acetate borane (55 g, 76.57%) as a yellow solid. LCMS (ESI): m/z [M+H] calc'd for C₂₁H₃₀BNO₄ 371.2; found 372.2.

### Step 4

To a stirred solution of 2,2-dimethyl-3-[5-(4,4,5,5-tetramethyl-1,3,2-dioxaborolan-2-yl)-1H-indol-3-yl]propyl acetate (54 g, 145.443 mmol) and methyl (2S)-3-(4-bromo-1,3-thiazol-2-yl)-2-[(tert-butoxycarbonyl) amino]propanoate (79.68 g, 218.1 mmol) and K₃PO₄ (77.18 g, 363.6 mmol) in toluene (330 mL) and dioxane (110 mL) and H₂O (110 mL) were added Pd(dppf)Cl₂ (10.64 g, 14.5 mmol) in portions at room temperature under argon atmosphere. The resulting mixture was stirred for 36 h at 70° C. under argon atmosphere. The resulting mixture was concentrated under vacuum. The resulting mixture was extracted with EtOAc (3 L). The combined organic layers were washed with water (3×2 L), dried over anhydrous Na₂SO₄. After filtration, the filtrate was concentrated under reduced pressure. The residue was purified by silica gel column chromatography, eluted with PE/EtOAc (1:1) to afford methyl (2S)-3-(4-[3-[3-(acetyloxy)-2,2-dimethylpropyl]-1H-indol-5-yl]-1,3-thiazol-2-yl)-2-[(tert-butoxycarbonyl) amino]propanoate (54 g, 60.78%) as a yellow oil. LCMS (ESI): m/z [M+H] calc'd for C₂₇H₃₅N₃O₆S 529.2; found 530.2.

### Step 5

To a stirred solution of methyl (2S)-3-(4-[3-[3-(acetyloxy)-2,2-dimethylpropyl]-1H-indol-5-yl]-1,3-thiazol-2-yl)-2-[(tert-butoxycarbonyl) amino]propanoate (54 g, 101.954 mmol) in THF (450 mL) were added NaHCO₃ (10.28 g, 122.3 mmol) and AgOTf (31.44 g, 122.3 mmol) dropwise at 0° C. To the stirred solution was added 12 (23.29 g, 91.6 mmol) in THF (100 mL) dropwise at 0° C. The resulting mixture was stirred for 15 min at 0° C. The reaction was quenched with sat. Na₂S203 (aq.) at 0° C. The resulting mixture was extracted with EtOAc (1 L). The combined organic layers were washed with water (3 L), dried over anhydrous Na₂SO₄. After filtration, the filtrate was concentrated under reduced pressure. The residue was purified by silica gel column chromatography, eluted with PE/EtOAc (5:1) to afford methyl (2S)-3-(4-[3-[3-(acetyloxy)-2,2-dimethylpropyl]-2-iodo-1H-indol-5-yl]-1,3-thiazol-2-yl)-2-[(tert-butoxycarbonyl)amino]propanoate (40 g, 53.80%) as a yellow solid. LCMS (ESI): m/z [M+H] calc'd for C₂₇H₃₄IN₃O₆S 655.1; found 656.1.

### Step 6

To a stirred solution of methyl (2S)-3-(4-[3-[3-(acetyloxy)-2,2-dimethylpropyl]-2-iodo-1H-indol-5-yl]-1,3-thiazol-2-yl)-2-[(tert-butoxycarbonyl) amino]propanoate (40 g, 61.01 mmol) in THF (300 mL) and H₂O (100 mL) were added LiOH (4.38 g, 183.05 mmol) dropwise at 0° C. The resulting mixture was stirred for overnight at room temperature. The residue was acidified to pH 6 with conc. HCl. The resulting mixture was extracted with EtOAc (500 mL). The combined organic layers were washed with water (3×500 mL), dried over anhydrous Na₂SO₄. After filtration, the filtrate was concentrated under reduced pressure. This resulted in (2S)-2-[(tert-butoxycarbonyl) amino]-3-[4-[3-(3-hydroxy-2,2-dimethylpropyl)-2-iodo-1H-indol-5-yl]-1, 3-thiazol-2-yl]propanoic acid (40 g, crude) as a yellow oil. The crude product was used in the next step directly without further purification. LCMS (ESI): m/z [M+H] calc'd for C₂₄H₃₀IN₃O₅S 599.1.1; found 600.1.

### Step 7

To a stirred solution of (2S)-2-[(tert-butoxycarbonyl) amino]-3-[4-[3-(3-hydroxy-2,2-dimethylpropyl)-2-iodo-1H-indol-5-yl]-1,3-thiazol-2-yl]propanoic acid (40 g, 66.72 mmol) and methyl (3S)-1,2-diazinane-3-carboxylate (28.86 g, 200.17 mmol) and HOBT (1.8 g, 13.35 mmol) and DIEA (172.47 g, 1334.5 mmol) in DCM (350 mL) were added EDCI (31.98 g, 166.8 mmol) dropwise at 0° C. The resulting mixture was stirred for overnight at room temperature under nitrogen atmosphere. The resulting mixture was washed with water (1.5 L), dried over anhydrous Na₂SO₄. After filtration, the filtrate was concentrated under reduced pressure. The residue was purified by silica gel column chromatography, eluted with PE/EtOAc (1:1) to afford methyl (3S)-1-[(2S)-2-[(tert-butoxycarbonyl) amino]-3-[4-[3-(3-hydroxy-2,2-dimethylpropyl)-2-iodo-1H-indol-5-yl]-1, 3-thiazol-2-yl]propanoyl]-1,2-diazinane-3-carboxylate (28 g, 43.9%) as a yellow oil. LCMS (ESI): m/z [M+H] calc'd for C₃₀H₄₀IN₅O₆S 725.1.1; found 726.1

### Step 8

To a stirred solution of methyl (3S)-1-[(2S)-2-[(tert-butoxycarbonyl) amino]-3-[4-[3-(3-hydroxy-2,2-dimethylpropyl)-2-iodo-1H-indol-5-yl]-1,3-thiazol-2-yl]propanoyl]-1,2-diazinane-3-carboxylate (28 g, 38.5 mmol) in THF (240 mL) were added LiOH (2.77 g, 115.7 mmol) in H₂O (80 mL) dropwise at 0° C. The resulting mixture was stirred for 2 h at room temperature. The mixture was acidified to pH 6 with conc. HCl. The resulting mixture was extracted with EtOAc (300 mL). The combined organic layers were washed with water (3×300 mL), dried over anhydrous Na₂SO₄. After filtration, the filtrate was concentrated under reduced pressure. This resulted in (3S)-1-[(2S)-2-[(tert-butoxycarbonyl) amino]-3-[4-[3-(3-hydroxy-2,2-dimethylpropyl)-2-iodo-1H-indol-5-yl]-1,3-thiazol-2-yl]propanoyl]-1,2-diazinane-3-carboxylic acid (25 g, crude) as a yellow oil. The crude product was used in the next step directly without further purification. LCMS (ESI): m/z [M+H] calc'd for C₂₉H₃₈IN₅O₆S 711.1; found 712.2.

### Step 9

To a stirred solution of (3S)-1-[(2S)-2-[(tert-butoxycarbonyl) amino]-3-[4-[3-(3-hydroxy-2,2-dimethylpropyl)-2-iodo-1H-indol-5-yl]-1,3-thiazol-2-yl]propanoyl]-1,2-diazinane-3-carboxylic acid (25 g, 35.13 mmol) and HOBT (23.74 g, 175.6 mmol) and DIPEA (136.21 g, 1053.9 mmol) in DCM (2 L) were added EDCI (188.5 g, 983.6 mmol) in portions at 0° C. The resulting mixture was stirred for overnight at room temperature under nitrogen atmosphere. The resulting mixture was washed with water (6 L), dried over anhydrous Na₂SO₄. After filtration, the filtrate was concentrated under reduced pressure. The residue was purified by silica gel column chromatography, eluted with PE/EtOAc (2:1) to afford tert-butyl ((6³S,4S,Z)-12-iodo-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (13 g, 45.88%) as a yellow solid. LCMS (ESI): m/z [M+H] calc'd for C₂₉H₃₆IN₅O₅S 693.1; found 694.0.

### Step 10

To a stirred mixture of tert-butyl ((6³S,4S,Z)-12-iodo-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (13 g, 18.7 mmol) and KOAc (6.44 g, 65.6 mmol) and s-Phos (2.31 g, 5.62 mmol) in toluene (120 mL) were added Pd₂(dba)₃ (2.06 g, 2.25 mmol) in portions at room temperature under argon atmosphere. To the stirred solution were added 4,4,5,5-tetramethyl-1,3,2-dioxaborolane (17.99 g, 140.5 mmol) dropwise at 0° C. under argon atmosphere. The resulting mixture was stirred for 3 h at 60° C. under argon atmosphere. The reaction was quenched with sat. NH₄Cl (aq.) at 0° C. The resulting mixture was concentrated under vacuum. The resulting mixture was extracted with EtOAc (200 mL). The combined organic layers were washed with water (3×300 mL), dried over anhydrous Na₂SO₄. After filtration, the filtrate was concentrated under reduced pressure. The residue was purified by silica gel column chromatography, eluted with PE/EtOAc (3:1) to afford tert-butyl ((6³S,4S,Z)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate (10 g, 68.6% yield) as a yellow solid. LCMS (ESI): m/z [M+H] calc'd for C₃₅H₄₈BN₅O₇S 693.3; found 694.4.

### Intermediate 4. Synthesis of(S)-3-bromo-5-iodo-2-(1-methoxyethyl) pyridine

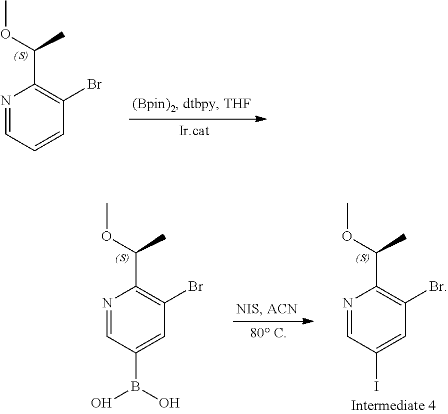

### Step 1

To a stirred solution of 3-bromo-2-[(1S)-1-methoxyethyl]pyridine (80.00 g, 370.24 mmol, 1.00 equiv) and bis (pinacolato)diboron (141.03 g, 555.3 mmol, 1.50 equiv) in THF (320 mL) was added dtbpy (14.91 g, 55.5 mmol) and Chloro (1,5-cyclooctadiene) iridium (I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL) and the mixture was adjusted to pH 10 with Na₂CO₃ (40 g) and NaOH (10 g) (mass 4:1) in water (600 mL). The aqueous layer was extracted with EtOAc (800 mL). The aqueous phase was acidified to pH=6 with HCl (6 N) to precipitate the desired solid to afford 5-bromo-6-[(1S)-1-methoxyethyl]pyridin-3-ylboronic acid (50 g, 52.0% yield) as a light-yellow solid. LCMS (ESI): m/z [M+H] calc'd for C₈H₁₁BBrNO₃ 259.0; found 260.0.

### Step 2

To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na₂S203 (3×500 mL). The organic layer was dried over anhydrous Na₂SO₄. After filtration, the filtrate was concentrated under reduced pressure. The residue was purified by silica gel column chromatography to afford(S)-3-bromo-5-iodo-2-(1-methoxyethyl) pyridine (20 g, 66.0% yield). LCMS (ESI): m/z [M+H] calc'd for C₈H₉BrINO 340.9; found 341.7.

### Intermediate 5. Synthesis of tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate

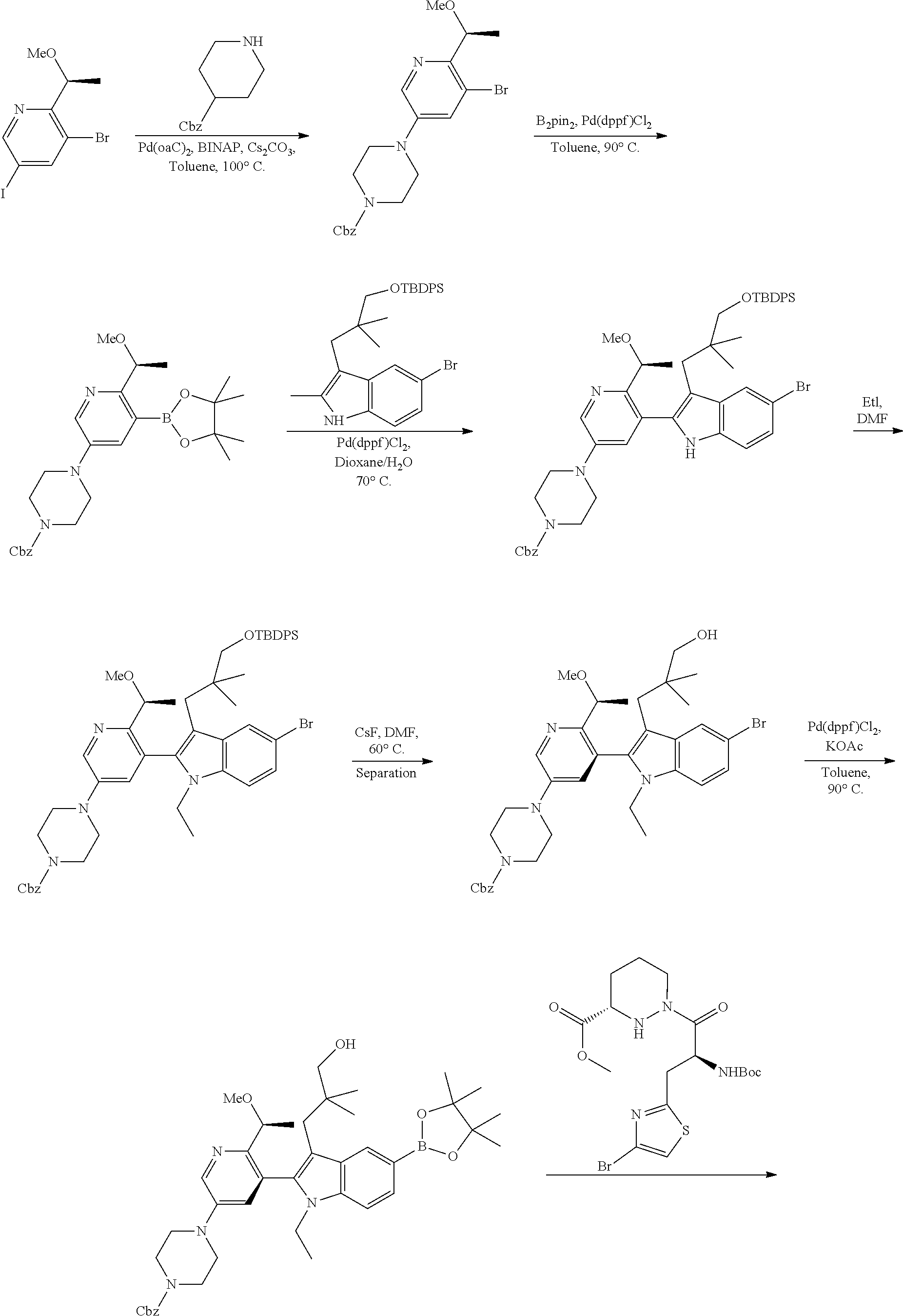

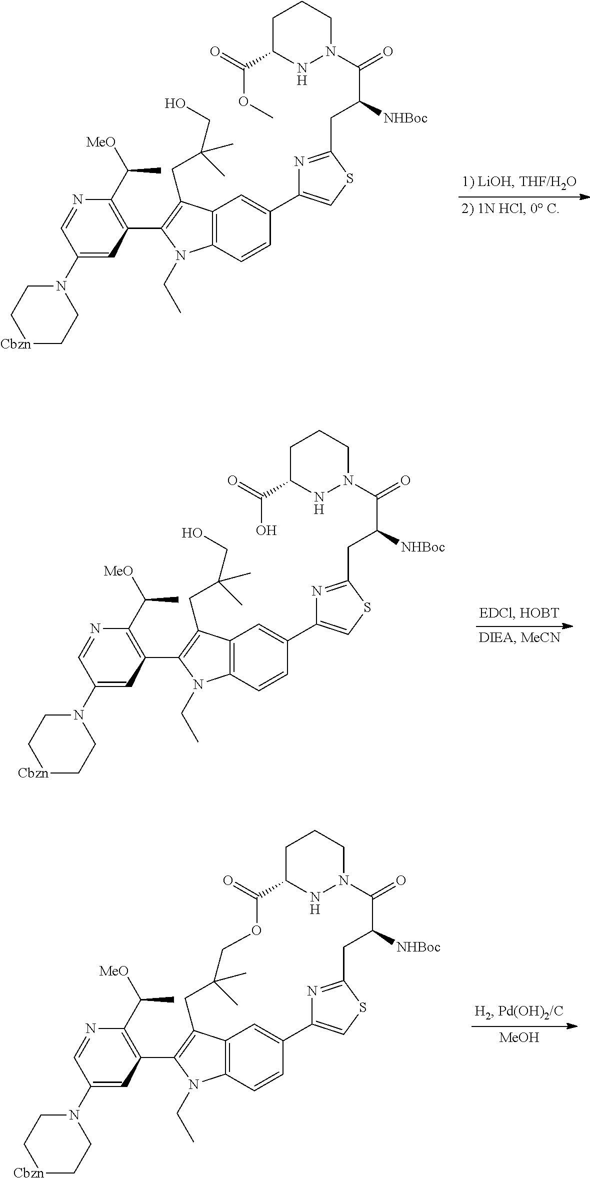

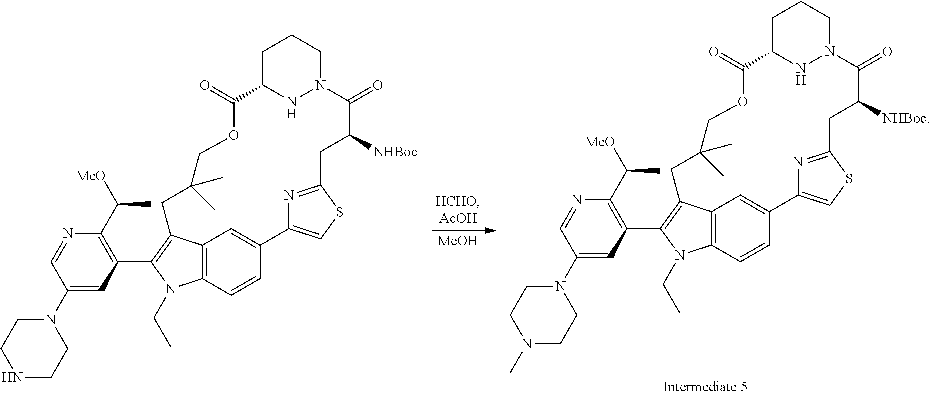

### Step 1

Into a 3L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd (OAc) 2 (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was concentrated under reduced pressure. The residue was applied onto a silica gel column with ethyl acetate/hexane (1:1). Removal of solvent under reduced pressure gave benzyl(S)-4-(5-bromo-6-(1-methoxyethyl) pyridin-3-yl) piperazine-1-carboxylate (135 g, 65.1% yield) as a dark yellow solid. LCMS (ESI): m/z [M+H] calc'd for C₂₀H₂₄BrN₃O₃ 433.1; found 434.1.

### Step 2

Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl(S)-4-(6-(1-methoxyethyl)-5-(4,4,5,5-tetramethyl-1,3,2-dioxaborolan-2-yl) pyridin-3-yl) piperazine-1-carboxylate (167 g, crude) as a dark yellow solid. LCMS (ESI): m/z [M+H] calc'd for C₂₆H₃₆BN₃O₅ 481.3; found 482.1.

### Step 3

Into a 3-L 3-necked round-bottom flask purged and maintained with an inert atmosphere of argon, was placed(S)-4-(6-(1-methoxyethyl)-5-(4,4,5,5-tetramethyl-1,3,2-dioxaborolan-2-yl) pyridin-3-yl) piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (146 g, 48.1% yield) as a yellow solid. LCMS (ESI): m/z [M+H] calc'd for C₄₉H₅₇BrN₄O₄Si 872.3; found 873.3.

### Step 4

To a stirred mixture of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (146 g, 167.0 mmol) and Cs₂CO₃ (163.28 g, 501.1 mmol) in DMF (1200 mL) was added C₂Hsl (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5L). The organic layers were dried over anhydrous Na₂SO₄. After filtration, the filtrate was concentrated under reduced pressure to give benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.

### Step 5

To a stirred mixture of benzyl benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl) pyridin-3-yl) piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1L). Then the organic phase was concentrated under reduced pressure. The residue was purified by silica gel column chromatography, eluted with PE/EA (1/3) to afford two atropisomers of benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (38 g, 36% yield, RT=1.677 min in 3 min LCMS (0.1% FA)) and B (34 g, 34% yield, RT=1.578 min in 3 min LCMS (0.1% FA)) both as yellow solid. LCMS (ESI): m/z [M+H] calc'd for C₃₅H₄₃BrN₄O₄ 663.2; found 662.2.

### Step 6

Into a 500-mL 3-necked round-bottom flask purged and maintained with an inert atmosphere of nitrogen, was placed benzyl(S)-4-(5-(5-bromo-1-ethyl-3-(3-hydroxy-2,2-dimethylpropyl)-1H-indol-2-yl)-6-(1-methoxyethyl) pyridin-3-yl) piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (1/3) to give benzyl(S) -4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (12 g, 76.0% yield) as a yellow solid. LCMS (ESI): m/z [M+H] calc'd for C₄₁H₅₅BN₄O₆ 710.4; found 711.3.

### Step 7

Into a 250-mL round-bottom flask purged and maintained with an inert atmosphere of argon, was placed benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (10.8 g, 15.2 mmol), methyl (3S)-1-[(2S)-3-(4-bromo-1,3-thiazol-2-yl)-2-[(tert-butoxycarbonyl) amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate (8 g, 50.9% yield) as a yellow solid. LCMS (ESI): m/z [M+H] calc'd for C₅₂H₆₈N₈O₉S 980.5; found 980.9.

### Step 8

To a stirred mixture of methyl(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylate (12 g, 12.23 mmol) in THF (100 mL)/H₂O (100 mL) was added LiOH (2.45 g, 61.1 mmol) under N₂ atmosphere and the resulting mixture was stirred for 2 h at 25° C. Desired product could be detected by LCMS. THF was concentrated under reduced pressure. The pH of aqueous phase was acidified to 5 with HCL (1N) at 0° C. The aqueous layer was extracted with DCM (3×100 ml). The organic phase was concentrated under reduced pressure to give (S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid (10 g, 84.5% yield) as a light yellow solid. LCMS (ESI): m/z [M+H] calc'd for C₅₁H₆₆N₈O₉S 966.5; found 967.0.

### Step 9

Into a 3-L round-bottom flask purged and maintained with an inert atmosphere of nitrogen, was placed(S)-1-((S)-3-(4-(2-(5-(4-((benzyloxy) carbonyl) piperazin-1-yl)-2-((S)-1-methoxyethyl) pyridin-3-yl)-1-ethyl-3-(3-hydroxy-2,2-dimethylpropyl)-1H-indol-5-yl) thiazol-2-yl)-2-((tert-butoxycarbonyl) amino) propanoyl) hexahydropyridazine-3-carboxylic acid (18 g, 18.61 mmol), ACN (1.8 L), DIEA (96.21 g, 744.4 mmol), EDCI (107.03 g, 558.3 mmol), HOBT (25.15 g, 186.1 mmol). The resulting solution was stirred for overnight at 25° C. The resulting mixture was concentrated under vacuum after reaction completed. The resulting solution was diluted with DCM (1 L). The resulting mixture was washed with HCl (3×1 L, 1N aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl) amino)-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl) piperazine-1-carboxylate (10.4 g, 54.8% yields) as a light yellow solid. LCMS (ESI): m/z [M+H] calc'd for C₅₁H₆₄N₈O₈S 948.5; found 949.3.

### Step 10

Into a 250-mL round-bottom flask purged and maintained with an inert atmosphere of nitrogen, was placed benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl) piperazine-1-carboxylate (10.40 g, 10.9 mmol), Pd (OH) 2/C(5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((6³S,4S,Z)-1¹-ethyl-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.5 g, 90.4% yield) as a light yellow solid. LCMS (ESI): m/z [M+H] calc'd for C₄₃H₅₈N₈O₆S 814.4; found 815.3.

### Step 11

Into a 1000-mL round-bottom flask purged and maintained with an inert atmosphere of nitrogen, was placed tert-butyl ((6³S,4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 ml water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate (8.2 g, 90.1% yield) as a yellow solid. LCMS (ESI): m/z [M+H] calc'd for C₄₄H₆₀N₈O₆S 828.4; found 829.3.

### Example A120. (1S,2S)—N-((6³S,4S,Z)-1¹-ethyl-1²-(5-((S)-hexahydropyrazino [2,1-c][1,4]oxazin-8 (1H)-yl)-2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

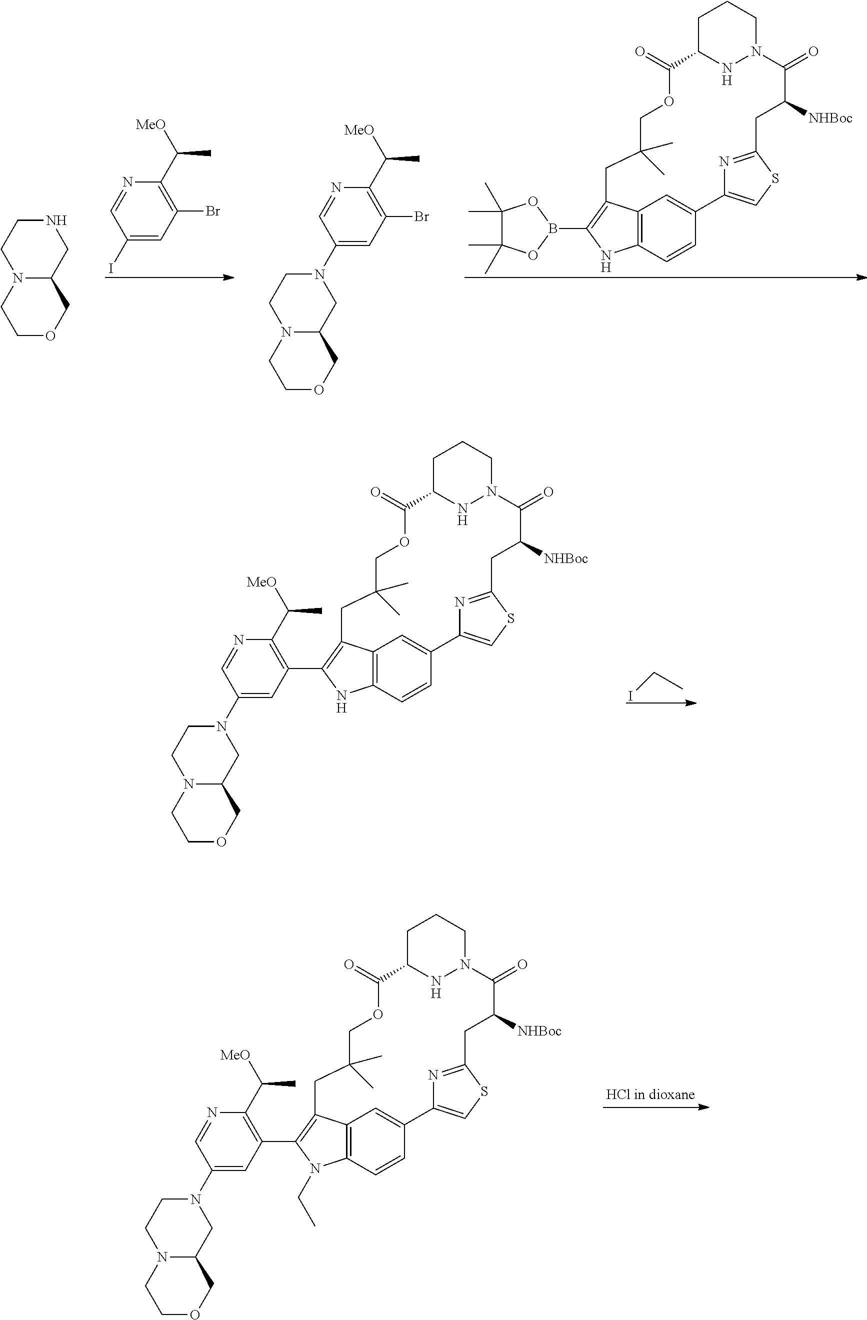

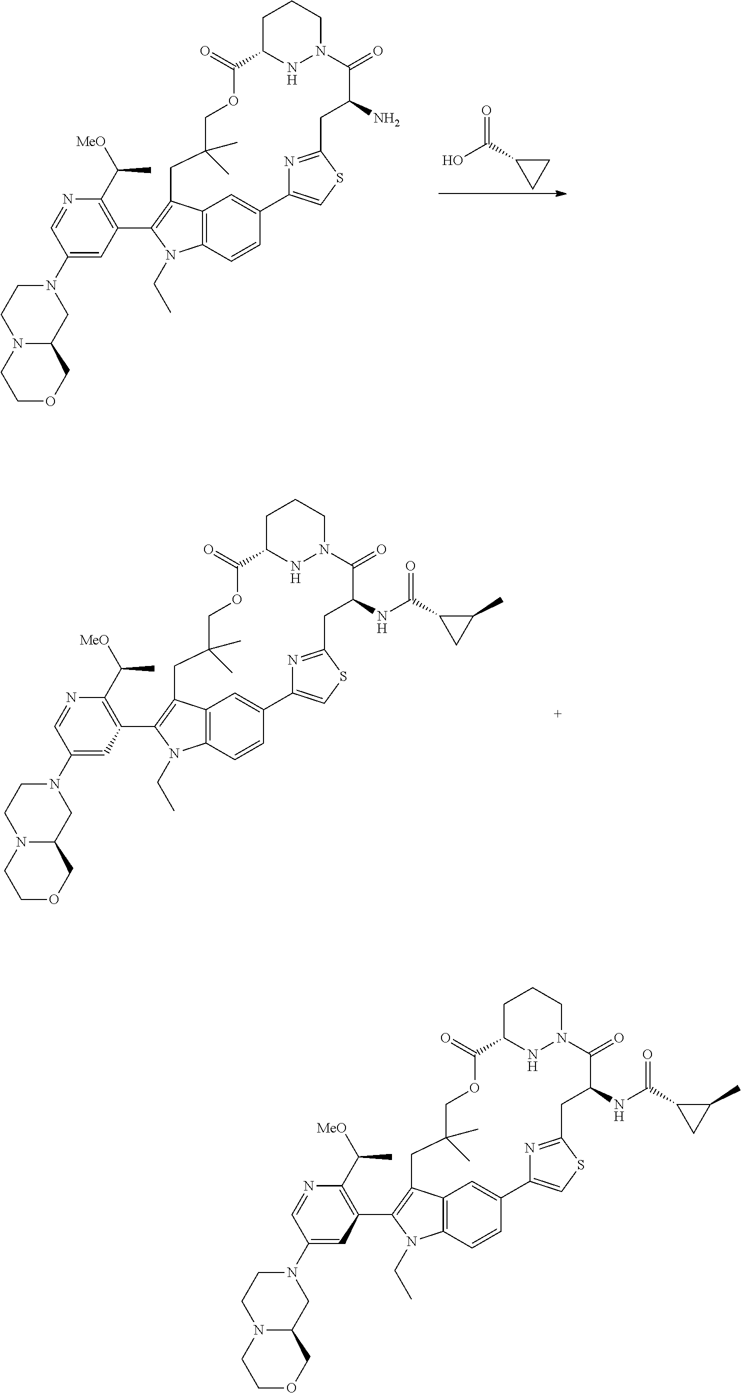

### Step 1

To a stirred solution of 3-bromo-5-iodo-2-[(1S)-1-methoxyethyl]pyridine (1 g, 2.92 mmol) (S)-octahydropyrazino [2,1-c][1,4]oxazine (498.9 mg, 3.1 mmol) and Potassium tert-butoxide (656.25 mg, 5.8 mmol) in Toluene (15 mL) were added Pd₂ (dba) 3 (53.55 mg, 0.06 mmol) and XantPhos (169.2 mg, 0.29 mmol) at room temperature under nitrogen atmosphere. The resulting mixture was stirred for 3 h at 100° C. under nitrogen atmosphere. After completion of reaction, the solution was concentrated under reduced pressure. The residue was purified by reverse flash chromatography (conditions: column, C18 silica gel; mobile phase, ACN in water (0.05% TFA), 0% to 100% gradient in 40 min; detector, UV 254 nm) to afford(S)-8-(5-bromo-6-((S)-1-methoxyethyl) pyridin-3-yl) octahydropyrazino [2,1-c][1,4]oxazine (670 mg, 57.2% yield) as a yellow solid. LCMS (ESI): m/z [M+H] calc'd for C₁₅H₂₂BrN₃O₂ 355.1; found 356.1.

### Step 2

To a mixture of(S)-8-(5-bromo-6-((S)-1-methoxyethyl) pyridin-3-yl) octahydropyrazino [2,1-c][1,4]oxazine (670 mg, 1.88 mmol), Intermediate 3 (1.56 g, 2.26 mmol) and K₂CO₃ (779.74 mg, 5.6 mmol) in Toluene (9 mL), H₂O (3 mL) and 1,4-dioxane (3 mL) was added Pd(dppf)Cl₂ (137.61 mg, 0.19 mmol) at room temperature under nitrogen atmosphere. The resulting mixture was stirred for overnight at 65° C. under nitrogen atmosphere. After completion of reaction, the solution was concentrated under reduced pressure. The residue was purified by reverse flash chromatography (conditions: column, C18 silica gel; mobile phase, ACN in water (0.05% TFA), 0% to 100% gradient in 30 min; detector, UV 254 nm) to afford tert-butyl ((6³S,4S, Z)-12-(5-((S)-hexahydropyrazino [2,1-c][1,4]oxazin-8 (1H)-yl)-2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (1.4 g, 88.3% yield) as a white solid. LCMS (ESI): m/z [M+H] calc'd for C₄₄H₅₈N₈O₇S 842.4; found 843.2.

### Step 3

To a stirred mixture of tert-butyl ((6³S,4S, Z)-12-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (1.4 g, 1.66 mmol) and Cs₂CO₃ (1.62 g, 4.97 mmol) in DMF (10 mL) was added ethyl iodide (0.39 g, 2.5 mmol) dropwise at 0° C. The resulting mixture was stirred for 2 h at room temperature. After completion of reaction, the solution was concentrated under reduced pressure. The residue was purified by reverse flash chromatography (conditions: column, C18 silica gel; mobile phase, ACN in water (0.05% TFA), 0% to 100% gradient in 30 min;

detector, UV 254 nm) to afford tert-butyl ((6³S,4S, Z)-1¹-ethyl-1²-(5-((S)-hexahydropyrazino [2,1-c][1,4]oxazin-8 (1H)-yl)-2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (800 mg, 49.7% yield) as a brown yellow solid. LCMS (ESI): m/z [M+H] calc'd for C₄₆H₆₂N₈O₇S 870.4; found 871.2.

### Step 4

Into a 50 mL round-bottom flask were added tert-butyl ((63 S, 4S, Z)-1¹-ethyl-1²-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (250 mg, 0.29 mmol) and HCl (4M in 1,4-dioxane, 10 mL) at 0° C. The resulting mixture was stirred for 1 h at room temperature. The resulting mixture was concentrated. The resulting mixture was diluted with 30 mL of dichloromethane and 20 ml saturated NaHCO₃ aqueous solution. The organic phase was washed twice with 30 mL brine. Removal of solvent under reduced pressure resulted in (6³S,4S,Z)-4-amino-1¹-ethyl-1²-(5-((S)-hexahydropyrazino [2, 1-c][1,4]oxazin-8 (1H)-yl)-2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (170.00 mg, crude) as a brown solid. LCMS (ESI): m/z [M+H] calc'd for C₄₁H₅₄N₈O₅S 770.4; found 771.2.

### Step 5

To a stirred solution of (6³S,4S,Z)-4-amino-1¹-ethyl-1²-(5-((S)-hexahydropyrazino [2,1-c][1,4]oxazin-8 (1H)-yl)-2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (170 mg, 0.22 mmol) in DMF (8 mL) were added DIEA (2.8 g, 22 mmol), (1S,2S)-2-methylcyclopropane-1-carboxylic acid (33 mg, 0.33 mmol) and HATU (125 mg, 0.33 mmol) at 0° C. The resulting mixture was stirred for 2 h at room temperature. After completion of reaction, the solution was concentrated under reduced pressure. The crude product was purified by Prep-HPLC to afford 50 mg racemated product. The racemate was purified by Prep-CHIRAL-HPLC with the following conditions (Column: CHIRAL ART Cellulose-SB, 2*25 cm, 5 μm; Mobile Phase A: MtBE (10 mM NH₃-MeOH), Mobile Phase B: EtOH; Flow rate: 20 mL/min; Gradient: 50% B to 50% B in 7 min; 275/210 nm) to afford two atropisomers of (1S,2S)—N-((6³S,4S,Z)-1¹-ethyl-1²-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide (as single atropisomer) (10.7 mg, 5.1% yield) and (6 mg, 3.03%) both as white solid. LCMS (ESI): m/z [M+H] calc'd for C₄₆H₆₀N₈O₆S 852.3; found 853.5. Isomer 1. ¹H NMR (400 MHZ, DMSO-d₆) δ 8.53 (d, 2H), 8.46 (d, 1H), 7.79 (s, 1H), 7.71 (d, 1H), 7.51 (d, 1H), 7.38 (d, 1H), 5.55 (t, 1H), 5.05 (d, 1H), 4.22 (t, 2H), 3.99-3.80 (m, 4H), 3.82-3.59 (m, 5H), 3.54 (d, 2H), 3.39 (d, 1H), 3.14 (t, 2H), 3.07 (s, 3H), 2.99 (s, 1H), 2.81 (t, 3H), 2.67 (d, 1H), 2.44-2.34 (m, 2H), 2.30 (s, 1H), 2.21-2.07 (m, 2H), 1.80 (s, 2H), 1.51 (s, 2H), 1.21 (d, 4H), 1.15-0.93 (m, 7H), 0.87 (s, 3H), 0.65 (m, 2H), 0.52 (s, 4H). Isomer 2. ¹H NMR (400 MHZ, DMSO-d₆) δ 8.54-8.39 (m, 3H), 7.79 (s, 1H), 7.75-7.67 (m, 1H), 7.55 (d, 1H), 7.22 (d, 1H), 5.56 (t, 1H), 5.07 (d, 1H), 4.34-4.09 (m, 5H), 3.83-3.62 (m, 4H), 3.55 (d, 3H), 3.21 (s, 3H), 3.14 (d, 2H), 2.94-2.64 (m, 5H), 2.46-2.36 (m, 2H), 2.32-2.15 (m, 3H), 2.08 (d, 1H), 1.79 (s, 2H), 1.49 (s, 2H), 1.33 (d, 3H), 1.25 (d, 1H), 1.06 (s, 4H), 0.90 (d, 7H), 0.54 (d, 1H), 0.34 (s, 3H).

### Example A14. N-[(7S,13S,19M)-21-ethyl-20-{2-[(1S)-1-methoxyethyl]pyridin-3-yl}-17,17-dimethyl-8,14-dioxo-15-oxa-4-thia-9,21,27,28-tetraazapentacyclo [17.5.2.1^{2,5}.1^{9,13}.0^{22,26}]octacosa-1 (25),2,5 (28), 19,22 (26),23-hexaen-7-yl]azetidine-3-carboxamide

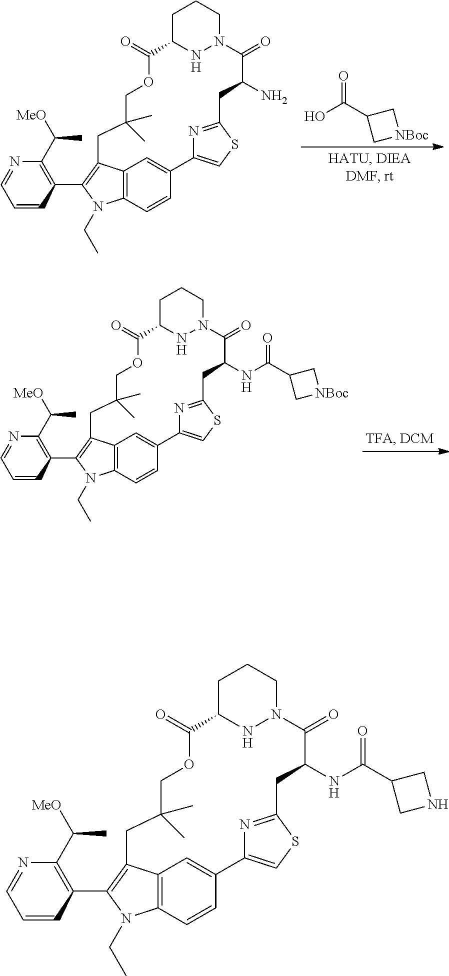

### Step 1

To a stirred solution of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (450.00 mg, 0.71 mmol, 1.00 equiv) and 1-(tert-butoxycarbonyl) azetidine-3-carboxylic acid (215.3 mg, 1.07 mmol) in DMF (5.00 mL) were added DIEA (460.99 mg, 3.5 mmol) and HATU (379.7 mg, 1 mmol) in portions at room temperature under N₂ atmosphere until the reaction was complete by LCMS. The resulting mixture was extracted with EtOAc (3×20 mL) and the combined organic layers were washed with brine (2×20 mL), dried over anhydrous Na₂SO₄. After filtration, the filtrate was concentrated under reduced pressure and the resulting residue was purified by silica gel column chromatography to afford tert-butyl 3-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamoyl) azetidine-1-carboxylate (520 mg, 90%) as a yellow solid. LCMS (ESI): m/z [M+H] calc'd for C₄₃H₅₅N₇O₇S ESI-MS 813.4; found: 814.4

### Step 2

To a solution of tert-butyl 3-(((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamoyl) azetidine-1-carboxylate (170.00 mg, 0.21 mmol) in DCM (1.6 mL) was added TFA (0.4 mL, 5.3 mmol) dropwised at 0° C. It was stirred for 2 h at room temperature under N₂ atmosphere and then concentrated under reduced pressure. The crude product was purified by Prep-HPLC to afford N-[(7S,13S,19M)-21-ethyl-20-{2-[(1S)-1-methoxyethyl]pyridin-3-yl}-17, 17-dimethyl-8, 14-dioxo-15-oxa-4-thia-9,21,27,28-tetraazapentacyclo [17.5.2.1^{2,5}.1^{9,13}.0^{22,26}]octacosa-1 (25),2,5 (28), 19,22 (26),23-hexaen-7-yl]azetidine-3-carboxamide (44.7 mg, 30% yield) as a white solid. LCMS (ESI): m/z [M+H] calc'd for C₃₈H₄₇N₇O₅S 713.3; found 714.1.

### Example A99. (1S,2S)—N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴, 6⁵,6⁶-hexahydro-1¹H-8-oxa-2 (4,2)-thiazola-1 (5,3)-indola-6 (1,3)-pyridazinacycloundecaphane-4-yl)-2-phenylcyclopropane-1-carboxamide

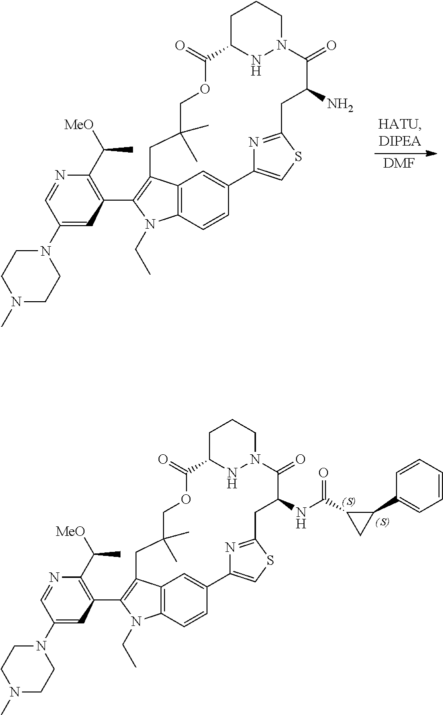

To a solution of tert-butyl ((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (50.0 mg, 0.069 mmol), (1S,2S)-2-phenylcyclopropane-1-carboxylic acid (16.69 mg, 0.103 mmol), and DIPEA (44.32 mg, 0.343 mmol) in DMF (0.50 mL) at 0° C. was added HATU (78.24 mg, 0.206 mmol). The resulting mixture was warmed to room temperature and stirred for 3 h. The crude product was purified by prep-HPLC to give (1S,2S)—N-((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-phenylcyclopropane-1-carboxamide (34 mg, 51% yield) as an off-white solid. LCMS (ESI): m/z [M+H] calc'd for C₄₉H₆₀N₈O₅S 873.5; found 874.1.

### Example A121. Synthesis of (1S,2S)—N-((6³S,4S,Z)-1¹-ethyl-1²-(2-((S)-1-methoxyethyl)-5-(4-(2-methoxyethyl) piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

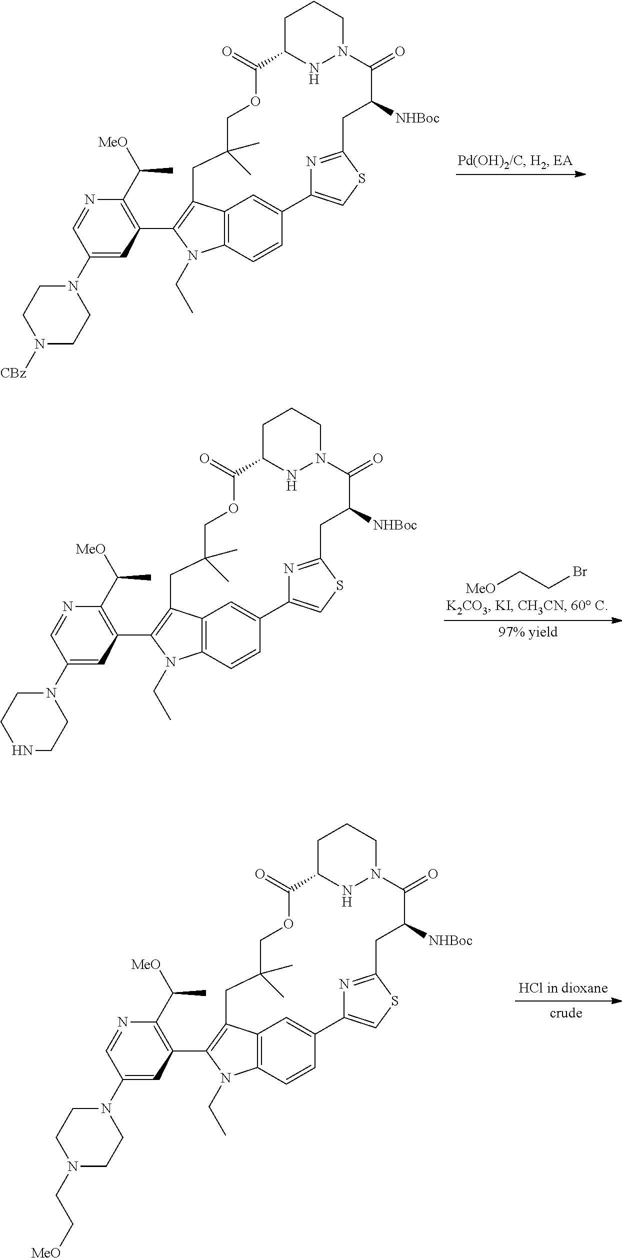

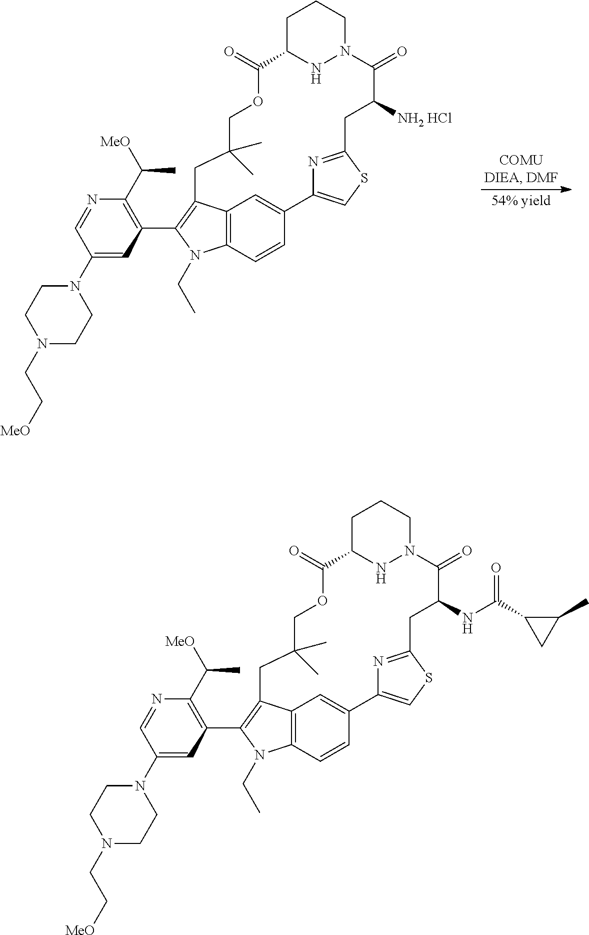

### Step 1

A mixture of benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl) piperazine-1-carboxylate (380 mg, 0.4 mmol) in EtOAc (10 mL) was added Pd (OH) 2/C(600 mg, 20 mol %) was hydrogenated at rt overnight. The mixture was filtered through a pad of Celite® and the filtrate was concentrated under reduced pressure to give tert-butyl ((6³S,4S,Z)-1¹-ethyl-1²-(2-((S)-1-methoxyethyl)-5-(piperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (310 mg, crude) as a solid. LCMS (ESI): m/z [M+H] calc'd for C₄₃H₅₈N₈O₆S 814.4; found 815.5.

### Step 2

To a mixture of tert-butyl ((6³S,4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (300 mg, 0.37 mmol) and 1-bromo-2-methoxyethane (56 mg, 0.41 mmol) in MeCN (10 mL) at rt was added KI (61 mg, 0.37 mmol) and K₂CO₃ (51 mg, 0.37 mmol) in portions. The mixture was heated to 60° C. and stirred for 2 h, then diluted with H₂O (5 mL). The residue was purified by preparative-HPLC to give tert-butyl ((6³S,4S,Z)-1¹-ethyl-1²-(2-((S)-1-methoxyethyl)-5-(4-(2-methoxyethyl) piperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate (310 mg, 97% yield) as a solid. LCMS (ESI): m/z [M+H] calc'd for C₄₆H₆₄N₈O₇S 872.5; found 873.6.

### Step 3

A mixture of tert-butyl ((6³S,4S,Z)-1¹-ethyl-1²-(2-((S)-1-methoxyethyl)-5-(4-(2-methoxyethyl) piperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (300 mg, 0.34 mmol) in 4 M HCl in 1,4-dioxane, (10 mL) was stirred at rt for 1 h, then concentrated under reduced pressure to give (6³S,4S,Z)-4-amino-1¹-ethyl-1²-(2-((S)-1-methoxyethyl)-5-(4-(2-methoxyethyl) piperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione HCl salt (315 mg, crude) as a solid, which was used directly in the next step without further purification. LCMS (ESI): m/z [M+H] calc'd for C₄₁H₅₆N₈O₆S 772.4; found 773.3.

### Step 4

A mixture of (6³S,4S,Z)-4-amino-1¹-ethyl-1²-(2-((S)-1-methoxyethyl)-5-(4-(2-methoxyethyl) piperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione HCl salt (300 mg, 0.39 mmol) and (1S,2S)-2-methylcyclopropane-1-carboxylic acid (97 mg, 0.97 mmol) in DMF (5 mL) at 0° C. was added DIPEA (1.00 g, 7.77 mmol) dropwise, then COMU (249 mg, 0.58 mmol) in portions. The mixture was allowed to warm to rt and stirred for 2 h, then concentrated under reduced pressure and the residue was purified by preparative-HPLC to give (1S,2S)—N-((6³S,4S,Z)-1¹-ethyl-1²-(2-((S)-1-methoxyethyl)-5-(4-(2-methoxyethyl) piperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide (178 mg, 54% yield) as a solid. LCMS (ESI): m/z [M+H] calc'd for C₄₆H₆₂N₈O₆S 854.5; found 855.5; ¹H NMR (400 MHZ, DMSO-d₆) δ 8.55-8.46 (m, 2H), 7.80 (d, J=3.1 Hz, 1H), 7.77-7.70 (m, 1H), 7.57 (d, J=8.6 Hz, 1H), 7.40 (d, J=2.8 Hz, 1H), 5.55 (d, J=9.2 Hz, 1H), 4.37-4.09 (m, 6H), 4.00 (s, 2H), 3.83-3.71 (m, 2H), 3.57 (s, 3H), 3.38 (t, J=4.8 Hz, 2H), 3.32 (d, J=2.5 Hz, 4H), 3.22 (s, 7H), 3.20-3.11 (m, 1H), 2.94 (d, J=14.4 Hz, 1H), 2.76 (t, J=11.4 Hz, 1H), 2.44 (d, J=14.2 Hz, 1H), 2.07 (d, J=12.0 Hz, 1H), 1.80 (s, 2H), 1.60-1.47 (m, 2H), 1.34 (d, J=6.1 Hz, 3H), 1.07 (d, J=1.8 Hz, 4H), 0.95-0.82 (m, 7H), 0.55 (d, J=7.4 Hz, 1H), 0.35 (s, 3H).

### Example A157. Synthesis of (1S,2S)—N-((6³S,4S,Z)-1¹-ethyl-1²-(2-((S)-1-methoxyethyl)-5-((R)-octahydro-2H-pyrido [1,2-a]pyrazin-2-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

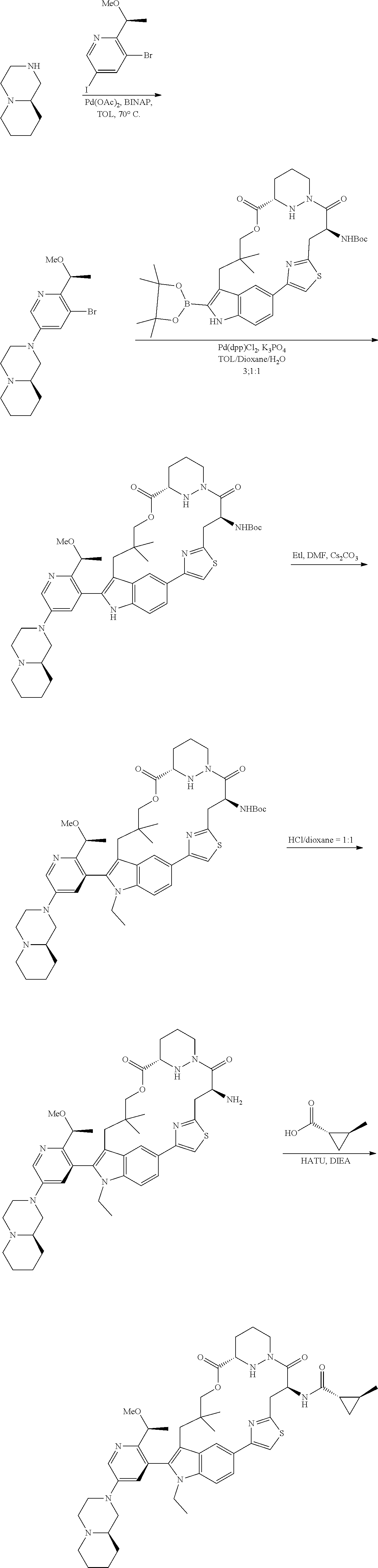

### Step 1

5-[(9aR)-octahydropyrido [1,2-a]pyrazin-2-yl]-3-bromo-2-[(1S)-1-methoxyethyl]pyridine was synthesized in a manner similar to(S)-8-(5-bromo-6-((S)-1-methoxyethyl) pyridin-3-yl) octahydropyrazino [2,1-c][1,4]oxazine except(S)-octahydropyrazino [2,1-c][1,4]oxazine was substituted with(S)-octahydropyrazino [2,1-c][1,4]oxazine (1.5 g, 60% yield) as a solid. LCMS (ESI): m/z [M+H] calc'd for C₁₆H₂₄BrN₃O 353.1; found 354.1.

### Step 2

Tert-butyl ((6³S,4S,Z)-12-(2-((S)-1-methoxyethyl)-5-((R)-octahydro-2H-pyrido [1,2-a]pyrazin-2-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate was synthesized in a manner similar to tert-butyl ((6³S,4S,Z)-12-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate except(S)-8-(5-bromo-6-((S)-1-methoxyethyl) pyridin-3-yl) octahydropyrazino [2,1-c][1,4]oxazine was substituted with 5-[(9aR)-octahydropyrido [1,2-a]pyrazin-2-yl]-3-bromo-2-[(1 S)-1-methoxyethyl]pyridine and K₂CO₃ was substituted with K₃PO₄ to give (800 mg, 83% yield) as an oil. LCMS (ESI): m/z [M+H] calc'd for C₄₅H₆₀N₈OBS 840.4; found 841.4.

### Step 3

Tert-butyl ((6³S,4S,Z)-1¹-ethyl-1²-(2-((S)-1-methoxyethyl)-5-((R)-octahydro-2H-pyrido [1,2-a]pyrazin-2-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate was synthesized in a manner similar to tert-butyl ((6³S,4S,Z)-1¹-ethyl-1²-(5-((S)-hexahydropyrazino [2,1-c][1,4]oxazin-8 (1H)-yl)-2-((S)-1-methoxyethyl) pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate except tert-butyl ((6³S,4S,Z)-12-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate was substituted with tert-butyl ((6³S,4S,Z)-12-(2-((S)-1-methoxyethyl)-5-((R)-octahydro-2H-pyrido [1,2-a]pyrazin-2-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate to give (220 mg, 27% yield) as a solid. LCMS (ESI): m/z [M+H] calc'd for C₄₇H₆₄N₈O₆S 868.5; found 869.5.

### Step 4

A mixture of tert-butyl ((6³S,4S,Z)-1¹-ethyl-1²-(2-((S)-1-methoxyethyl)-5-((R)-octahydro-2H-pyrido [1,2-a]pyrazin-2-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (220 mg, 0.25 mmol) in 1,4-dioxane (2 mL) at 0° C. was added 4M HCl in 1,4-dioxane (1 mL). The mixture was stirred at 0° C. for 1 h then concentrated under reduced pressure to give (6³S,4S,Z)-4-amino-11-ethyl-1²-(2-((S)-1-methoxyethyl)-5-((R)-octahydro-2H-pyrido [1,2-a]pyrazin-2-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (220 mg, crude) as a solid. LCMS (ESI): m/z [M+H] calc'd for C₄₂H₅₆N₈O₄S 768.4; found 769.4.

### Step 5

(1S,2S)—N-((6³S,4S,Z)-1¹-ethyl-1²-(2-((S)-1-methoxyethyl)-5-((R)-octahydro-2H-pyrido [1,2-a]pyrazin-2-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide was synthesized in a manner similar to (1S,2S)—N-((6³S,4S,Z)-1¹-ethyl-1²-(5-((S)-hexahydropyrazino [2,1-c][1,4]oxazin-8 (1H)-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide except (6³S,4S,Z)-4-amino-1¹-ethyl-1²-(5-((S)-hexahydropyrazino [2,1-c][1,4]oxazin-8 (1H)-yl)-2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione HCl salt was substituted with (6³S,4S,Z)-4-amino-1¹-ethyl-1²-(2-((S)-1-methoxyethyl)-5-((R)-octahydro-2H-pyrido [1,2-a]pyrazin-2-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione to give (13 mg, 8% yield) as a solid. LCMS (ESI): m/z [M+H] calc'd for C₄₇H₆₂N₈O₅S 850.5; found 851.6; ¹H NMR (400 MHZ, DMSO-d₆) δ 8.53-8.41 (m, 3H), 7.79 (s, 1H), 7.72-7.70 (m, 1H), 7.55-7.30 (m, 1H), 7.20-7.10 (m, 1H), 5.56-5.46 (m, 1H), 5.08-5.00 (m, 1H), 4.39-4.04 (m, 5H), 3.72-7.62 (m, 2H), 3.57-3.47 (m, 2H), 3.21-3.11 (m, 3H), 3.15-3.08 (m, 1H), 2.94 (m, 1H), 2.79-2.69 (m, 4H), 2.45-2.35 (m, 3H), 2.24-2.22 (m, 1H), 2.08-2.00 (m, 1H), 2.01-1.88 (m, 2H), 1.81-1.65 (m, 3H), 1.59 (d, J=12.1 Hz, 2H), 1.54-1.38 (m, 2H), 1.33-1.30 m, 3H), 1.28-1.12 (m, 3H), 1.06-0.86 (m, 4H), 0.96-0.79 (m, 6H), 0.55-0.50 (m, 1H), 0.34 (s, 3H).

### Example A214. Synthesis of (1S,2S)—N-((6³S,4S,Z)-11-(2-cyanopropan-2-yl)-10,10-dimethyl-1²-(2-(4-methylpiperazin-1-yl) pyridin-4-yl)-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2-methylcyclopropane-1-carboxamide

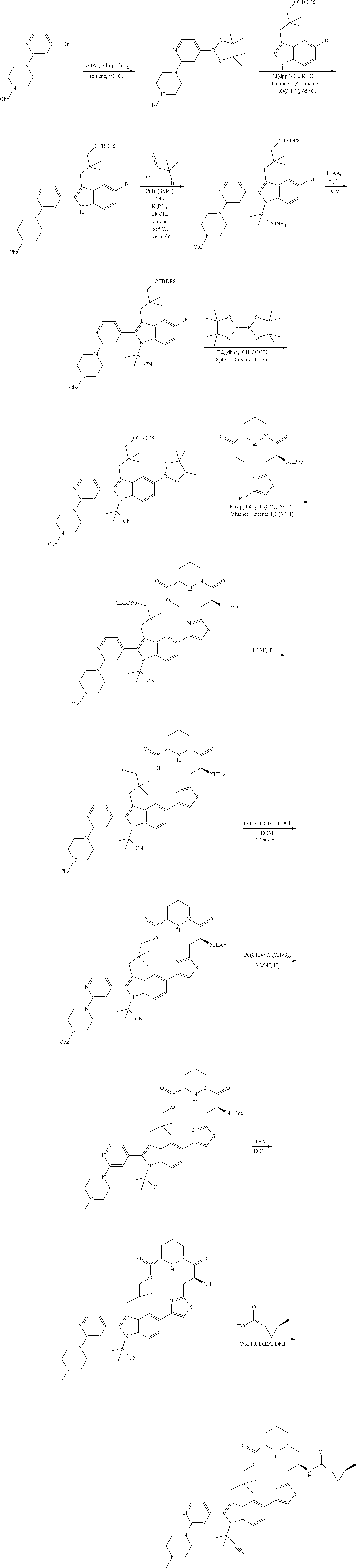

### Step 1

A mixture of benzyl 4-(4-bromopyridin-2-yl) piperazine-1-carboxylate (8.09 g, 21.5 mmol), bis (pinacolato)diboron (8.19 g, 32.3 mmol), KOAc (6.33 g, 64.5 mmol), Pd(dppf)Cl₂ (0.79 g, 1.1 mmol) in toluene (100 mL) under an atmosphere of Ar was heated to 90° C. and stirred for 2 h. The mixture was concentrated under vacuum, H₂O (50 mL) was added to the residue and the mixture was extracted with EtOAc (2×100 mL). The combined organic layers were washed with brine (2×100 mL), dried, filtered and the filtrate concentrated under reduced pressure to give benzyl 4-[4-(4,4,5,5-tetramethyl-1,3,2-dioxaborolan-2-yl) pyridin-2-yl]piperazine-1-carboxylate (9.2 g, 100% yield) as a solid. LCMS (ESI): m/z [M+H] calc'd for C₂₃H₃₀BN₃O₄ 423.2; found 424.2.

### Step 2

A mixture of benzyl 4-[4-(4,4,5,5-tetramethyl-1,3,2-dioxaborolan-2-yl) pyridin-2-yl]piperazine-1-carboxylate (5.00 g, 11.8 mmol), 5-bromo-3-[3-[(tert-butyldiphenylsilyl)oxy]-2,2-dimethylpropyl]-2-iodo-1H-indole (7.64 g, 11.8 mmol), Pd(dppf)Cl₂ (0.86 g, 1.2 mmol), K₂CO₃ (6.27 g, 45.4 mmol) in toluene (45 mL), 1,4-dioxane (15 mL), H₂O (15 mL) under an atmosphere of N₂ was heated to 70° C. and stirred for 2 h. H₂O (50 mL) was added and the mixture was extracted with EtOAc (2×50 mL). The combined organic layers were dried over anhydrous Na₂SO₄ and filtered. The filtrate was concentrated under reduced pressure and the residue was purified by silica gel column chromatography to give benzyl 4-[4-(5-bromo-3-[3-[(tert-butyldiphenylsilyl)oxy]-2,2-dimethylpropyl]-1H-indol-2-yl) pyridin-2-yl]piperazine-1-carboxylate (4.9 g, 51% yield) as solid. LCMS (ESI): m/z [M+H] calc'd for C₄₆H₅₁BrN₄O₃Si 814.3; found 815.4.

### Step 3

A mixture of benzyl 4-[4-(5-bromo-3-[3-[(tert-butyldiphenylsilyl)oxy]-2,2-dimethylpropyl]-1H-indol-2-yl) pyridin-2-yl]piperazine-1-carboxylate (4.5 g, 5.5 mmol), 2-bromo-2-methylpropanamide (2.75 g, 16.6 mmol), K₃PO₄ (2.34 g, 11.0 mmol), NaOH (0.57 g, 14.3 mmol), Ph₃P (0.29 g, 1.1 mmol), copper bromide-dimethyl sulfide (0.23 g, 1.1 mmol) in toluene (50 mL) under an atmosphere of N₂ was heated to 45° C. and stirred for 2 days. H₂O (50 mL) was added and the mixture was extracted with EtOAc (2×50 mL). The combined organic layers were washed with brine (2×50 mL), dried over Na₂SO₄ and filtered. The filtrate was concentrated under reduced pressure and the residue was purified by silica gel column chromatography to give benzyl 4-[4-(5-bromo-3-[3-[(tert-butyldiphenylsilyl)oxy]-2,2-dimethylpropyl]-1-(1-carbamoyl-1-methylethyl) indol-2-yl) pyridin-2-yl]piperazine-1-carboxylate (1.5 g, 30% yield) as a solid. LCMS (ESI): m/z [M+H] calc'd for C₅₀H₅₈BrN₅O₄Si 899.3; found 900.4.

### Step 4

A mixture of benzyl 4-[4-(5-bromo-3-[3-[(tert-butyldiphenylsilyl)oxy]-2,2-dimethylpropyl]-1-(1-carbamoyl-1-methylethyl) indol-2-yl) pyridin-2-yl]piperazine-1-carboxylate (1.40 g, 1.6 mmol), Et₃N (0.47 g, 4.7 mmol) and TFAA (0.65 g, 3.1 mmol) in DCM (20 mL), 3 equiv) was stirred at rt for 2 h. H₂O (20 mL) was added and the mixture was extracted with DCM (2×20 mL). The combined organic layers were washed with brine (2×50 mL), dried over Na₂SO₄ and filtered. The filtrate was concentrated under reduced pressure to give benzyl 4-[4-(5-bromo-3-[3-[(tert-butyldiphenylsilyl)oxy]-2,2-dimethylpropyl]-1-(1-cyano-1-methylethyl) indol-2-yl) pyridin-2-yl]piperazine-1-carboxylate (1.3 g, 95% yield) as a solid. LCMS (ESI): m/z [M+H] calc'd for C₅₀H₅₆BrN₅O₃Si 881.3; found 882.4.

### Step 5

A mixture of benzyl 4-[4-(5-bromo-3-[3-[(tert-butyldiphenylsilyl)oxy]-2,2-dimethylpropyl]-1-(1-cyano-1-methylethyl) indol-2-yl) pyridin-2-yl]piperazine-1-carboxylate (1.50 g, 1.7 mmol), bis (pinacolato)diboron (5.21 g, 20.5 mmol), Pd₂ (dba) 3 (0.38 g, 0.4 mmol), KOAc (1.21 g, 12.3 mmol), X-Phos (0.20 g, 0.4 mmol) in 1,4-dioxane (25 mL) under an atmosphere of N₂ was heated to 110° C. and stirred for 2 h. H₂O (25 mL) was added and the mixture was extracted with EtOAc (2×25 mL). The combined organic layers were washed with brine (2×25 mL), dried over Na₂SO₄ and filtered. The filtrate was concentrated under reduced pressure and the residue was purified by silica gel column chromatography to give benzyl 4-[4-(3-[3-[(tert-butyldiphenylsilyl)oxy]-2,2-dimethylpropyl]-1-(1-cyano-1-methylethyl)-5-(4,4,5,5-tetramethyl-1,3,2-dioxaborolan-2-yl) indol-2-yl) pyridin-2-yl]piperazine-1-carboxylate (1.6 g, 94% yield) as an oil. LCMS (ESI): m/z [M+H] calc'd for C₅₆H₆₈BN₅O₅Si 929.5; found 930.4.

### Step 6

A mixture of benzyl 4-[4-(3-[3-[(tert-butyldiphenylsilyl)oxy]-2,2-dimethylpropyl]-1-(1-cyano-1-methylethyl)-5-(4,4,5,5-tetramethyl-1,3,2-dioxaborolan-2-yl) indol-2-yl) pyridin-2-yl]piperazine-1-carboxylate (1.60 g, 1.7 mmol), methyl (3S)-1-[(2S)-3-(4-bromo-1,3-thiazol-2-yl)-2-[(tert-butoxycarbonyl) amino]propanoyl]-1,2-diazinane-3-carboxylate (0.82 g, 1.7 mmol), K₂CO₃ (0.79 g, 5.8 mmol), Pd(dppf)Cl₂ (0.13 g, 0.17 mmol) in toluene (12 mL), 1,4-dioxane (4 mL) and H₂O (4 mL) under an atmosphere of N₂ was heated to 70° C. and stirred for 5 h. H₂O (30 mL) was added and the mixture was extracted with EtOAc (2×30 mL). The combined organic layers were washed with brine (2×30 mL), dried over Na₂SO₄ and filtered. The filtrate was concentrated under reduced pressure and the residue was purified by silica gel column chromatography to give benzyl 4-[4-(5-[2-[(2S)-2-[(tert-butoxycarbonyl) amino]-3-[(3S)-3-(methoxycarbonyl)-1,2-diazinan-1-yl]-3-oxopropyl]-1,3-thiazol-4-yl]-3-[3-[(tert-butyldiphenylsilyl)oxy]-2,2-dimethylpropyl]-1-(1-cyano-1-methylethyl) indol-2-yl) pyridin-2-yl]piperazine-1-carboxylate (650 mg, 31% yield) as an oil. LCMS (ESI): m/z [M+H] calc'd for C₆₇H₈₁N₉O₈SSi 1199.6; found 1200.5.

### Step 7

To a mixture of benzyl 4-[4-(5-[2-[(2S)-2-[(tert-butoxycarbonyl) amino]-3-[(3S)-3-(methoxycarbonyl)-1, 2-diazinan-1-yl]-3-oxopropyl]-1,3-thiazol-4-yl]-3-[3-[(tert-butyldiphenylsilyl)oxy]-2,2-dimethylpropyl]-1-(1-cyano-1-methylethyl) indol-2-yl) pyridin-2-yl]piperazine-1-carboxylate (650 mg, 0.54 mmol) in THF (15 mL) under an atmosphere of N₂ was added TBAF (1.42 g, 5.4 mmol). The mixture was stirred at rt overnight then the mixture adjusted to PH ˜6 with 1M HCl and extracted with EtOAc (2×50 mL). The combined organic layers were washed with brine (2×50 mL), dried over Na₂SO₄ and filtered. The filtrate was concentrated under reduced pressure and the residue was purified by silica gel column chromatography to give (3S)-1-[(2S)-3-[4-[2-(2-[4-[(benzyloxy) carbonyl]piperazin-1-yl]pyridin-4-yl)-1-(1-cyano-1-methylethyl)-3-(3-hydroxy-2,2-dimethylpropyl) indol-5-yl]-1,3-thiazol-2-yl]-2-[(tert-butoxycarbonyl) amino]propanoyl]-1,2-diazinane-3-carboxylic acid (370 mg, 72% yield) as a solid. LCMS (ESI): m/z [M+H] calc'd for C₅₀H₆₁N₉O₈S 947.4; found 948.5.

### Step 8

A mixture of (3S)-1-[(2S)-3-[4-[2-(2-[4-[(benzyloxy) carbonyl]piperazin-1-yl]pyridin-4-yl)-1-(1-cyano-1-methylethyl)-3-(3-hydroxy-2,2-dimethylpropyl) indol-5-yl]-1,3-thiazol-2-yl]-2-[(tert-butoxycarbonyl) amino]propanoyl]-1,2-diazinane-3-carboxylic acid (370 mg, 0.39 mmol), DIPEA (1.51 g, 11.7 mmol), HOBT (264 mg, 1.95 mmol), EDCI (2.09 g, 10.9 mmol) in DCM (370 mL) was stirred at rt overnight. H₂O (100 mL) was added and the mixture was extracted with EtOAc (2×100 mL). The combined organic layers were washed with brine (2×100 mL), dried over Na₂SO₄, filtered and the filtrate concentrated under reduced pressure to give benzyl 4-(4-((6³S,4S,Z)-4-((tert-butoxycarbonyl) amino)-11-(2-cyanopropan-2-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl) pyridin-2-yl) piperazine-1-carboxylate (187 mg, 52% yield) as a solid. LCMS (ESI): m/z [M+H] calc'd for C₅₀H₅₉N₉OS 929.4; found 930.8.

### Step 9

A mixture of benzyl 4-(4-((6³S,4S,Z)-4-((tert-butoxycarbonyl) amino)-11-(2-cyanopropan-2-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl) pyridin-2-yl) piperazine-1-carboxylate (170 mg, 0.18 mmol), paraformaldehyde (165 mg, 1.8 mmol), Pd (OH) 2/C(170 mg, 1.2 mmol) in MeOH (25 mL) was stirred under an atmosphere of H₂ overnight. The mixture was filtered and the filtrate was concentrated under reduced pressure to give tert-butyl ((6³S,4S,Z)-11-(2-cyanopropan-2-yl)-10,10-dimethyl-1²-(2-(4-methylpiperazin-1-yl) pyridin-4-yl)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (110 mg, 74% yield) as a solid. LCMS (ESI): m/z [M+H] calc'd for C₄₃H₅₅N₉O₅S 809.4; found 810.9.

### Step 10

A mixture of tert-butyl ((6³S,4S,Z)-11-(2-cyanopropan-2-yl)-10, 10-dimethyl-1²-(2-(4-methylpiperazin-1-yl) pyridin-4-yl)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (110 mg, 0.14 mmol) and TFA (5.0 mL, 67.3 mmol) in DCM (5 mL) was stirred at rt for 1 h, then concentrated under reduced pressure to give 2-((6³S,4S,Z)-4-amino-10, 10-dimethyl-1²-(2-(4-methylpiperazin-1-yl) pyridin-4-yl)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-11-yl)-2-methylpropanenitrile (96 mg, 100% yield) as an oil. LCMS (ESI): m/z [M+H] calc'd for C₃₈H₄₇N₉O₃S 709.4; found 710.5.

### Step 11

A mixture of 2-((6³S,4S,Z)-4-amino-10, 10-dimethyl-1²-(2-(4-methylpiperazin-1-yl) pyridin-4-yl)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-11-yl)-2-methylpropanenitrile (110 mg, 0.16 mmol), (1S,2S)-2-methylcyclopropane-1-carboxylic acid (47 mg, 0.47 mmol), COMU (66 mg, 0.16 mmol), DIPEA (1.00 g, 7.75 mmol) in DMF (5 mL) was stirred at rt for 2 h. H₂O (10 mL) was added and the mixture was extracted with EtOAc (2×10 mL). The combined organic layers were washed with brine (2×10 mL), dried over Na₂SO₄ and filtered. The filtrate was concentrated under reduced pressure and the residue was purified by preparative-HPLC to give (1S,2S)—N-((6³S,4S,Z)-11-(2-cyanopropan-2-yl)-10, 10-dimethyl-1²-(2-(4-methylpiperazin-1-yl) pyridin-4-yl)-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2-methylcyclopropane-1-carboxamide (21 mg, 17% yield) as a solid. LCMS (ESI): m/z [M+H] calc'd for C₄₃H₅₃N₉O₄S 791.4; found 792.4; 1H-NMR (400 MHZ, DMSO-d₆) δ 8.51 (d, J=7.1 Hz, 2H), 8.21 (d, J=5.0 Hz, 1H), 8.16 (d, J=5.0 Hz, 1H), 7.89 (d, J=1.8 Hz, 1H), 7.83 (d, J=1.5 Hz, 2H), 7.06 (s, 1H), 6.96 (s, 1H), 6.74-6.68 (m, 1H), 5.54 (q, J=8.6 Hz, 1H), 5.04 (d, J=12.2 Hz, 1H), 4.20 (q, J=12.4 Hz, 2H), 3.66 (dd, J=16.0, 10.9 Hz, 1H), 3.55 (s, 5H), 3.50 (d, J=10.9 Hz, 1H), 3.35 (s, 1H), 3.15 (dd, J=14.8, 9.2 Hz, 1H), 2.93 (dd, J=14.4, 6.3 Hz, 1H), 2.77 (s, 1H), 2.38 (dd, J=10.9, 5.5 Hz, 4H), 2.20 (d, J=5.3 Hz, 3H), 2.09 (s, 1H), 2.06 (s, 1H), 2.09-1.99 (m, 3H), 1.82-1.51 (d, J=4.0 Hz, 4H), 1.07 (s, 4H), 0.90-0.86 (d, J=3.0 Hz, 4H), 0.55 (d, J=6.8 Hz, 1H), 0.47-0.38 (m, 3H).

### Examples A221 and A222. Synthesis of (1S,2S)-2-(difluoromethyl)-N-((6³S,4S,Z)-11-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) cyclopropane-1-carboxamide and (1R,2R)-2-(difluoromethyl)-N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) cyclopropane-1-carboxamide

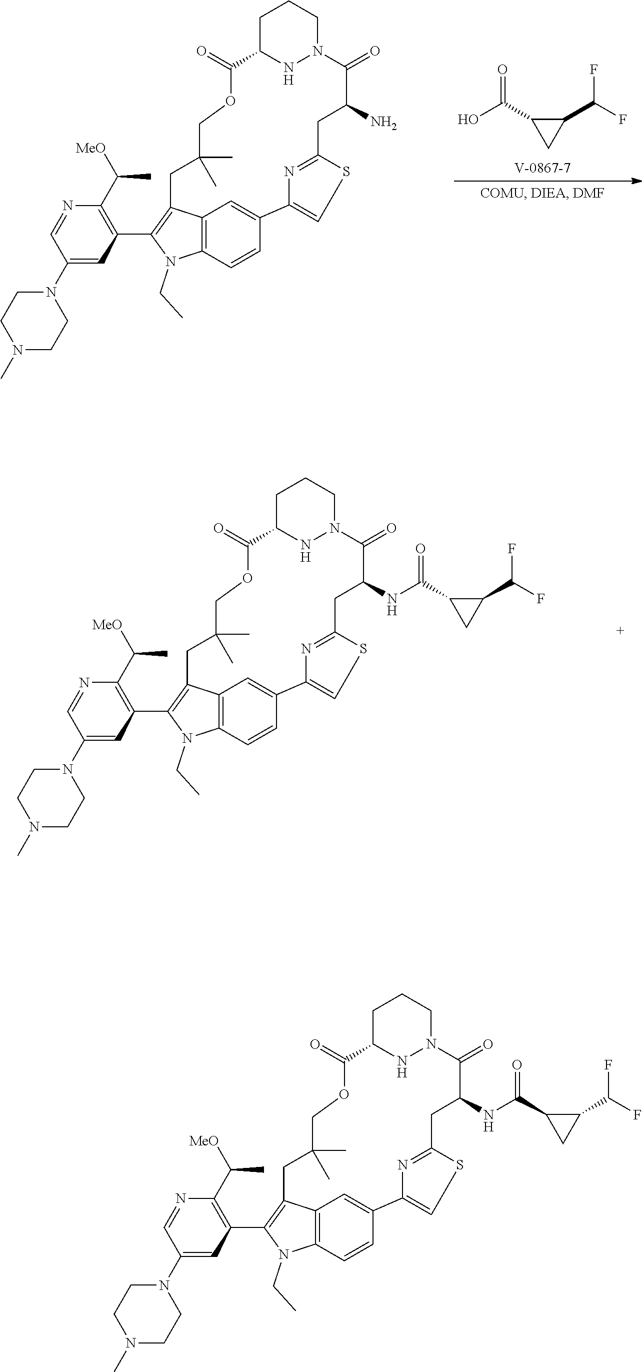

### Step 1

To a mixture of (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (200 mg, 0.27 mmol) and trans-2-(difluoromethyl)cyclopropane-1-carboxylic acid (56 mg, 0.41 mmol) in DMF (8 mL) at 0° C. was added DIPEA (177 mg, 1.37 mmol) dropwise, followed by COMU (235 mg, 0.55 mmol). The mixture was allowed to warm to rt and stirred for 1 h, then diluted with EtOAc (10 mL) and H₂O (50 mL). The aqueous and organic layers were separated and the aqueous layer was extracted with EtOAc (3×10 mL). The combined organic layers were washed with brine (2×10 mL), dried over anhydrous Na₂SO₄ and filtered. The filtrate was concentrated under reduced pressure and the residue was purified by preparative-HPLC to give (1S,2S)-2-(difluoromethyl)-N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) cyclopropane-1-carboxamide (32 mg, 27% yield) and (1R,2R)-2-(difluoromethyl)-N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) cyclopropane-1-carboxamide (31 mg, 27% yield) both as solids. LCMS (ESI): m/z [M+H] calc'd for C₄₄H₅₆F₂N₈O₅S 846.4; found 847.3; ¹H NMR (400 MHZ, DMSO-d₆) δ 8.80 (s, 1H), 8.46 (dd, J=13.4, 2.2 Hz, 2H), 7.83 (s, 2H), 7.73 (dd, J=8.7, 1.6 Hz, 1H), 7.56 (d, J=8.6 Hz, 1H), 7.23 (d, J=2.8 Hz, 1H), 5.95 (d, J=5.3 Hz, 1H), 5.57 (t, J=9.1 Hz, 1H), 5.12 (d, J=12.2 Hz, 1H), 4.14 (d, J=6.4 Hz, 5H), 3.57 (s, 2H), 3.21 (s, 4H), 2.93 (d, J=14.3 Hz, 2H), 2.78-2.68 (m, 1H), 2.67 (p, J=1.9 Hz, 3H), 2.46-2.28 (m, 3H), 2.25 (s, 2H), 2.17-1.92 (m, 2H), 1.79 (s, 2H), 1.66 (dt, J=9.7, 5.0 Hz, 1H), 1.51 (t, J=9.0 Hz, 1H), 1.33 (d, J=6.1 Hz, 4H), 1.24 (d, J=5.6 Hz, 2H), 0.96 (s, 1H), 0.95-0.72 (m, 6H), 0.35 (s, 3H) and LCMS (ESI): m/z [M+H] calc'd for C₄₄H₅₆F₂N₈O₅S 846.4; found 847.3; ¹H NMR (400 MHZ, DMSO-d₆) δ 8.87 (d, J=9.0 Hz, 1H), 8.46 (dd, J=11.3, 2.2 Hz, 2H), 7.80 (s, 2H), 7.73 (dd, J=8.7, 1.6 Hz, 1H), 7.56 (d, J=8.7 Hz, 1H), 7.23 (d, J=2.9 Hz, 1H), 6.09 (d, J=5.1 Hz, 1H), 5.94 (d, J=5.1 Hz, 1H), 5.80 (d, J=5.2 Hz, 1H), 5.59 (t, J=9.1 Hz, 1H), 5.13 (d, J=12.2 Hz, 1H), 4.50-4.06 (m, 5H), 3.57 (s, 2H), 3.21 (s, 4H), 2.93 (d, J=14.1 Hz, 2H), 2.67 (p, J=1.9 Hz, 4H), 2.39-2.15 (m, 4H), 2.17-1.82 (m, 2H), 1.77 (d, J=16.1 Hz, 2H), 1.44 (d, J=43.3 Hz, 1H), 1.35-1.24 (m, 4H), 1.15-0.91 (m, 2H), 0.91 (s, 6H), 0.34 (s, 3H).

### Example A173. Synthesis of (2S)—N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2,4-dimethylpiperazine-1-carboxamide

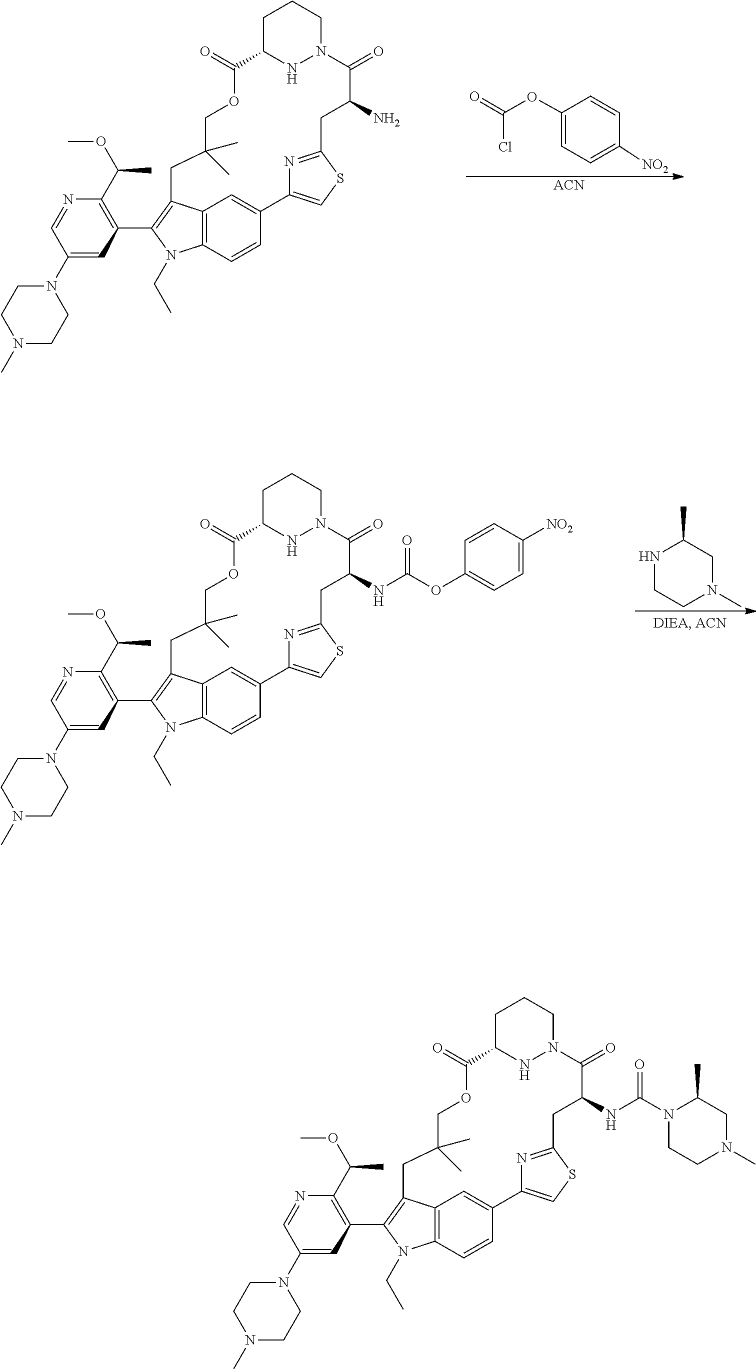

### Step 1

To a mixture of (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (80 mg, 0.11 mmol) in DCM (6 mL) at 0° C. was added pyridine (2 mL), then 4-nitrophenyl carbonochloridate (55 mg, 0.28 mmol) in portions. The mixture was allowed to warm to rt and stirred for 1 h at room temperature, then washed with 1M NaHSO₄ (10 mL) and H₂O (10 mL). The organic layer was concentrated under reduced pressure. to give 4-nitrophenyl ((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (98 mg, crude) as a solid. LCMS (ESI): m/z [M+H] calc'd for C₄₆H₅₅N₉O₈S 893.4; found 894.2.

### Step 2

To a mixture of 4-nitrophenyl ((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (98 mg, 0.11 mmol) and(S)-1,3-dimethylpiperazine (63 mg, 0.55 mmol) in ACN (5 mL) at 0° C. was added DIPEA (43 mg, 0.33 mmol) in ACN (2 mL. The crude product was purified by preparative-HPLC to give (2S)—N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2,4-dimethylpiperazine-1-carboxamide (13 mg, 13% yield) as a solid. LCMS (ESI): m/z [M+H] calc'd for C₄₆H₆₄N₁₀O₅S 868.5; found 869.3; ¹H NMR (400 MHZ, DMSO-d₆) δ 8.47 (s, 2H), 7.80 (s, 1H), 7.73 (s, 1H), 7.55 (s, 1H), 7.23 (s, 1H), 6.81-6.70 (m, 1H), 5.33-5.25 (m, 1H), 4.99 (s, 1H), 4.40-3.97 (m, 6H), 3.72 (s, 1H), 3.61-3.47 (m, 3H), 3.31-3.22 (m, 8H), 3.02-2.72 (m, 5H), 2.66 (s, 1H), 2.60-2.51 (m, 3H), 2.49-2.37 (m, 2H), 2.35-2.13 (m, 6H), 2.12-1.97 (m, 2H), 1.95-1.66 (m, 3H), 1.55 (s, 1H), 1.33 (s, 3H), 1.27-1.19 (m, 4H), 0.99-0.82 (m, 6H), 0.33 (s, 3H).

### Example A225. Synthesis of (1S,2S)—N-((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

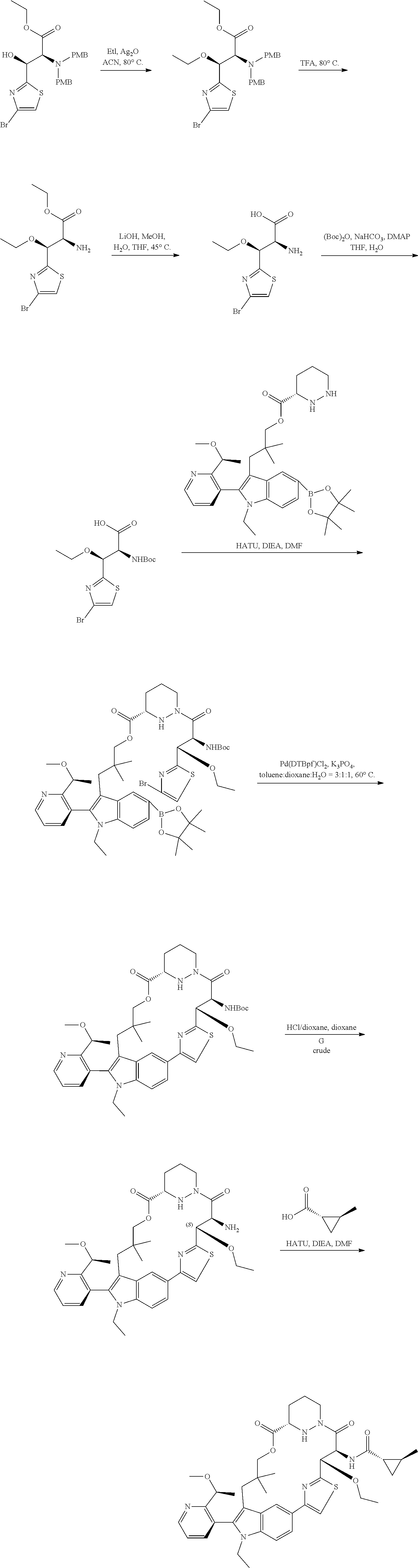

### Step 1

To a mixture of ethyl (2S,3S)-2-[bis [(4-methoxyphenyl)methyl]amino]-3-(4-bromo-1,3-thiazol-2-yl)-3-hydroxypropanoate (1.00 g, 1.9 mmol) and Ag2O (4.33 g, 18.7 mmol) in ACN (10 mL) at 0° C. under an atmosphere of N₂ was added ethyl iodide (2.91 g, 18.7 mmol) dropwise. The mixture was heated to 80° C. and stirred for 4 h, then filtered and the filter cake was washed with ACN (3×5 mL). The filtrate was concentrated under reduced pressure and the residue was purified by silica gel column chromatography to give ethyl (2S,3S)-2-[bis [(4-methoxyphenyl)methyl]amino]-3-(4-bromo-1,3-thiazol-2-yl)-3-ethoxypropanoate (557 mg, 53% yield) as an oil. LCMS (ESI): m/z [M+H] calc'd for C₂₆H₃₁BrN₂O₅S 562.1; found 563.2.

### Step 2

Into a 40 mL sealed tube were added ethyl (2S,3S)-2-[bis [(4-methoxyphenyl)methyl]amino]-3-(4-bromo-1,3-thiazol-2-yl)-3-ethoxypropanoate (530 mg) and TFA (10 mL) under an atmosphere of N₂. The mixture was heated to 80° C. and stirred overnight, then concentrated under reduced pressure to give ethyl (2S,3S)-2-amino-3-(4-bromo-1,3-thiazol-2-yl)-3-ethoxypropanoate that was used directly in the next step without further purification. LCMS (ESI): m/z [M+H] calc'd for C₁₀H₁₅BrN₂O₃S 322.0; found 323.0.

### Step 3

A mixture of ethyl (2S,3S)-2-amino-3-(4-bromo-1,3-thiazol-2-yl)-3-ethoxypropanoate (890 mg, 2.8 mmol), LiOH·H₂O (1.16 g, 27.6 mmol), MeOH (6 mL), THF (2 mL) and H₂O (2 mL) was stirred at 45° C. for 2 h. The mixture was concentrated under reduced pressure to give (2S,3S)-2-amino-3-(4-bromo-1,3-thiazol-2-yl)-3-ethoxypropanoic acid (that was used directly in the next step without further purification. LCMS (ESI): m/z [M+H] calc'd for C₈H₁₁BrN₂O₃S 294.0; found 294.9.

### Step 4

To a mixture of (2S,3S)-2-amino-3-(4-bromo-1,3-thiazol-2-yl)-3-ethoxypropanoic acid (890 mg, 3.0 mmol), NaHCO₃ (507 mg, 6.0 mmol) and DMAP (37 mg, 0.3 mmol) in THF/H₂O (1:1) at 0° C. was added (Boc) 20 (1.97 g, 9.0 mmol). The mixture was warmed to rt and stirred overnight then concentrated under reduced pressure to remove THF and the residue was acidified to PH ˜6 with HCl. The mixture was extracted with EtOAc (3×5 mL) and the combined organic layers were dried over anhydrous Na₂SO₄ and filtered. The filtrate was concentrated under reduced pressure and the residue was purified by silica gel column chromatography to give (2S,3S)-3-(4-bromo-1,3-thiazol-2-yl)-2-[(tert-butoxycarbonyl) amino]-3-ethoxypropanoic acid (369 mg, 31% yield) as a solid. LCMS (ESI): m/z [M+H] calc'd for C₁₃H₁₉BrN₂O₅S 394.0; found 395.0.

### Step 5

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-hexahydropyridazine-3-carboxylate (584 mg, 0.91 mmol) and (2S,3S)-3-(4-bromo-1,3-thiazol-2-yl)-2-[(tert-butoxycarbonyl) amino]-3-ethoxypropanoic acid (360 mg, 0.91 mmol) in DMF at 0° C. was added DIPEA (1.59 mL, 9.1 mmol) and HATU (693 mg, 1.8 mmol). The mixture was allowed to warm to rt and stirred for 1 h at room temperature, then cooled to 0° C. and H₂O added. The mixture was extracted with EtOAc (2×5 mL) and the combined organic layers were washed with brine (3×10 mL), dried over anhydrous Na₂SO₄ and filtered. The filtrate was concentrated under reduced pressure and the residue was purified by silica gel column chromatography to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((2S,3S)-3-(4-bromothiazol-2-yl)-2-((tert-butoxycarbonyl) amino)-3-ethoxypropanoyl) hexahydropyridazine-3-carboxylate (410 mg, 46%) yield as a solid. LCMS (ESI): m/z [M+H] calc'd for C₄₇H₆₆BBrN₆O₉S 980.4; found 981.3.

### Step 6

Into a 50 mL Schlenk tube were added 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((2S,3S)-3-(4-bromothiazol-2-yl)-2-((tert-butoxycarbonyl) amino)-3-ethoxypropanoyl) hexahydropyridazine-3-carboxylate (390 mg, 0.4 mmol), Pd(DTBpf)Cl₂ (78 mg, 0.12 mmol), K₃PO₄ (211 mg, 1.0 mmol), toluene (9 mL), 1,4-dioxane (3 mL) and H₂O (3 mL) under an atmosphere of Ar. The mixture was heated to 60° C. and stirred for 1 h, then extracted with EtOAc (3×15 mL). The combined organic layers were dried over anhydrous Na₂SO₄, filtered, the filtrate was concentrated under reduced pressure and the residue was purified by silica gel column chromatography to give tert-butyl ((6³S,3S,4S,Z)-3-ethoxy-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (96 mg, 31% yield) as a solid. LCMS (ESI): m/z [M+H] calc'd for C₄₁H₅₄N₆O₇S 774.4; found 775.4.

### Step 7

A mixture of tert-butyl ((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (92 mg, 0.12 mmol), HCl in 1,4-dioxane (2.5 mL) and 1,4-dioxane (2.5 mL) was stirred at rt for 1 h. The mixture was concentrated under reduced pressure to give tert-butyl ((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate (100 mg), which was used directly in the next step without further purification. LCMS (ESI): m/z [M+H] calc'd for C₃₆H₄₆N₆O₅S 674.3; found 675.3.

### Step 8

To a mixture of tert-butyl ((6³S,3S,4S,Z)-3-ethoxy-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (90 mg, 0.13 mmol) and (1S,2S)-2-methylcyclopropane-1-carboxylic acid (27 mg, 0.27 mmol) in DMF at 0° C. was added DIPEA (172 mg, 1.3 mmol) and HATU (101 mg, 0.27 mmol). The mixture was allowed to warm to rt and stirred for 1 h, then cooled to 0° C., H₂O added and the mixture extracted with EtOAc (2×5 mL). The combined organic layers were washed with brine (3×20 mL), dried over anhydrous Na₂SO₄ and filtered. The filtrate was concentrated under reduced pressure and the residue was purified by preparative-HPLC to give (1S,2S)—N-((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide (21 mg, 21% yield) as a solid. LCMS (ESI): m/z [M+H] calc'd for C₄₁H₅₂N₆O₆S 756.4; found 757.6; ¹H NMR (400 MHZ, DMSO-d₆) δ 8.76 (dd, J=4.8, 1.7 Hz, 1H), 8.50 (s, 1H), 7.91 (s, 1H), 7.84-7.71 (m, 3H), 7.61-7.56 (m, 1H), 7.55-7.50 (m, 1H), 5.91-5.85 (m, 1H), 5.20-5.14 (m, 1H), 4.92 (s, 1H), 4.36-4.21 (m, 3H), 4.17-4.07 (m, 2H), 3.68-3.56 (m 3H), 3.54-3.46 (m, 1H), 3.22 (s, 3H), 2.90-2.73 (m, 2H), 2.09-2.03 (m, 1H), 1.88-1.73 (m, 3H), 1.55-1.43 (m, 1H), 1.37 (d, J=6.0 Hz, 3H), 1.20 (t, J=6.9 Hz, 3H), 1.04 (s, 4H), 0.93-0.71 (m, 7H), 0.51 (d, J=6.3 Hz, 1H), 0.38 (s, 3H).

### Example A227. Synthesis of (1R,2R,3S)—N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

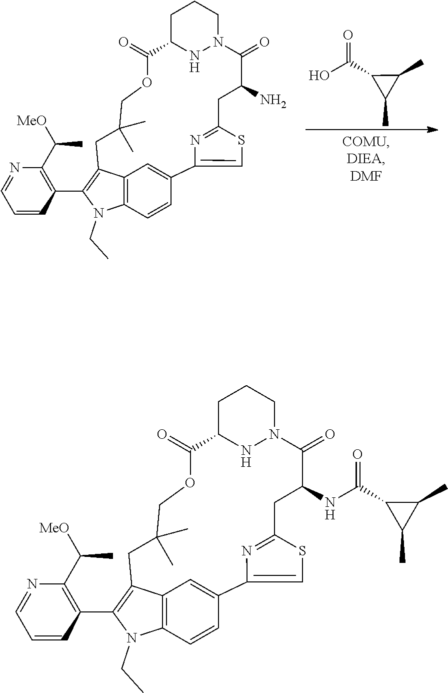

### Step 1

To a mixture of (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (126 mg, 0.20 mmol) and (1R,2R,3S)-2,3-dimethylcyclopropane-1-carboxylic acid (34 mg, 0.30 mmol) in DMF (5 mL) at 0° C. was added DIPEA (129 mg, 1.0 mmol) and COMU (171 mg, 0.4 mmol). The mixture was warmed to rt and stirred for 1 h, then diluted with EtOAc (20 mL) and H₂O (20 mL). The aqueous layer was extracted with EtOAc (3×10 mL) and the combined organic layers were washed with brine (2×10 mL), dried over anhydrous Na₂SO₄ and filtered. The filtrate was concentrated under reduced pressure and the residue was purified by preparative-HPLC to give (1R,2R,3S)—N-((6³S,4S,Z)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (29 mg, 25% yield) as a solid. LCMS (ESI): m/z [M+H] calc'd for C₄₀H₅₀N₆O₅S 726.4; found 727.3; ¹H NMR (400 MHZ, DMSO-d₆) δ 8.75 (dd, J=4.8, 1.7 Hz, 1H), 8.49 (d, J=1.6 Hz, 1H), 8.40 (d, J=9.0 Hz, 1H), 7.82 (s, 3H), 7.58 (d, J=8.6 Hz, 1H), 7.52 (dd, J=7.7, 4.7 Hz, 1H), 5.56 (t, J=9.0 Hz, 1H), 5.07 (d, J=12.2 Hz, 1H), 4.44-3.99 (m, 5H), 3.57 (s, 1H), 3.25 (s, 1H), 3.16 (d, J=9.3 Hz, 3H), 2.94 (d, J=14.3 Hz, 1H), 2.81-2.70 (m, 1H), 2.67 (p, J=1.9 Hz, 1H), 2.44-2.27 (m, 1H), 2.16-2.01 (m, 1H), 1.78 (s, 2H), 1.53 (s, 1H), 1.37 (d, J=6.0 Hz, 3H), 1.26-1.13 (m, 3H), 1.07 (dd, J=9.4, 5.4 Hz, 6H), 0.93-0.77 (m, 6H), 0.32 (s, 3H).

The following table of compounds were prepared using the aforementioned methods or variations thereof, as would be known to those of skill in the art.

TABLE 4
Exemplary Compounds Prepared by Methods of the Present Invention

| Ex# | LCMS (ESI): m/z [M + H] Found |
|---|---|
| A1 | 799.2 |
| A2 | 827.5 |
| A3 | 827.5 |
| A4 | 809.5 |
| A5 | 757.4 |
| A6 | 757.4 |
| A7 | 756.4 |
| A8 | 784.2 |
| A9 | 770.2 |
| A10 | 836.2 |
| A11 | 836.3 |
| A12 | 738.3 |
| A13 | 738.3 |
| A14 | 714.1 |
| A15 | 853.4 |
| A16 | 853.2 |
| A17 | 869.2 |
| A18 | 869.2 |
| A19 | 880.3 |
| A20 | 826.2 |
| A21 | 894.1 |
| A22 | 840.2 |
| A23 | 757.4 |
| A24 | 820.2 |
| A25 | 800.3 |
| A26 | 786.4 |
| A27 | 786.4 |
| A28 | 875.6 |
| A29 | 865.5 |
| A30 | 823.5 |
| A31 | 823.5 |
| A32 | 853.5 |
| A33 | 853.5 |
| A34 | 806.8 |
| A35 | 757.7 |
| A36 | 811.7 |
| A37 | 763.7 |
| A38 | 820.4 |
| A39 | 800.5 |
| A40 | 882.2 |
| A41 | 881.9 |
| A42 | 804.6 |
| A43 | 804.6 |
| A44 | 798.2 |
| A45 | 798.2 |
| A46 | 784.3 |
| A47 | 796.4 |
| A48 | 796.4 |
| A49 | 768.4 |
| A50 | 818.2 |
| A51 | 796.2 |
| A52 | 785.2 |
| A53 | 837.5 |
| A54 | 837.6 |
| A55 | 841.5 |
| A56 | 861.2 |
| A57 | 833.1 |
| A58 | 881.4 |
| A59 | 827.2 |
| A60 | 776.7 |
| A61 | 776.7 |
| A62 | 776.7 |
| A63 | 776.7 |
| A64 | 757.7 |
| A65 | 775.7 |
| A66 | 713.6 |
| A67 | 769.7 |
| A68 | 749.7 |
| A69 | 803.7 |
| A70 | 803.7 |
| A71 | 770.7 |
| A72 | 770.8 |
| A73 | 790.8 |
| A74 | 790.8 |
| A75 | 787.7 |
| A76 | 787.8 |
| A77 | 782.8 |
| A78 | 777.8 |
| A79 | 777.8 |
| A80 | 776.7 |
| A81 | 776.37 |
| A82 | 771.8 |
| A83 | 771.8 |
| A84 | 770 |
| A85 | 758.7 |
| A86 | 757.7 |
| A87 | 755.7 |
| A88 | 738.7 |
| A89 | 738.7 |
| A90 | 741.7 |
| A91 | 725.7 |
| A92 | 738.7 |
| A93 | 724.7 |
| A94 | 743.7 |
| A95 | 729.8 |
| A96 | 759.5 |
| A97 | 759.4 |
| A98 | 867.9 |
| A99 | 874.1 |
| A100 | 815.9 |
| A101 | 768.5 |
| A102 | 743.4 |
| A103 | 756.5 |
| A104 | 771.5 |
| A105 | 801.6 |
| A106 | 771.2 |
| A107 | 797.6 |
| A108 | 724.4 |
| A109 | 724.4 |
| A110 | 800.4 |
| A111 | 784.4 |
| A112 | 784.4 |
| A113 | 796.4 |
| A114 | 784.4 |
| A115 | 791.4 |
| A116 | 791.4 |
| A117 | 743.4 |
| A118 | 825.5 |
| A119 | 825.5 |
| A120 | 853.5 |
| A121 | 855.5 |
| A122 | 811.5 |
| A123 | 811.6 |
| A124 | 865.5 |
| A125 | 724.3 |
| A126 | 867.4 |
| A127 | 813.5 |
| A128 | 713.2 |
| A129 | 742.3 |
| A130 | 742.3 |
| A131 | 757.3 |
| A132 | 772.4 |
| A133 | 738.3 |
| A134 | 738.3 |
| A135 | 836.3 |
| A136 | 836.2 |
| A137 | 770.2 |
| A138 | 784.2 |
| A139 | 756.4 |
| A140 | 757.4 |
| A141 | 757.4 |
| A142 | 770.4 |
| A143 | 809.5 |
| A144 | 827.5 |
| A145 | 827.5 |
| A146 | 400.4 |
| A147 | 835.5 |
| A148 | 835.5 |
| A149 | 862.6 |
| A150 | 862.5 |
| A151 | 804 |
| A152 | 804.5 |
| A153 | 818.5 |
| A154 | 818.5 |
| A155 | 756.4 |
| A156 | 799.4 |
| A157 | 851.6 |
| A158 | 771 |
| A159 | 812.5 |
| A160 | 850.6 |
| A161 | 892.6 |
| A162 | 743.4 |
| A163 | 856.5 |
| A164 | 711.4 |
| A165 | 795.8 |
| A166 | 875.6 |
| A167 | 823.5 |
| A168 | 837.7 |
| A169 | 825.6 |
| A170 | 822.5 |
| A171 | 822.7 |
| A172 | 822.5 |
| A173 | 869.3 |
| A174 | 917.7 |
| A175 | 917.7 |
| A176 | 746.4 |
| A177 | 728.2 |
| A178 | 730.2 |
| A179 | 852.4 |
| A180 | 850.5 |
| A181 | 886.6 |
| A182 | 829 |
| A183 | 829 |
| A184 | 852.1 |
| A185 | 727.2 |
| A186 | 727.2 |
| A187 | 924.6 |
| A188 | 924.6 |
| A189 | 856.6 |
| A190 | 765.7 |
| A191 | 756.8 |
| A192 | 760.8 |
| A193 | 790.8 |
| A194 | 742.7 |
| A195 | 742.6 |
| A196 | 742.4 |
| A197 | 742.9 |
| A198 | 783 |
| A199 | 864.6 |
| A200 | 864.5 |
| A201 | 904.7 |
| A202 | 891.7 |
| A203 | 898.9 |
| A204 | 790.4 |
| A205 | 823.7 |
| A206 | 823.8 |
| A207 | 841.6 |
| A208 | 841.6 |
| A209 | 853.8 |
| A210 | 764.4 |
| A211 | 764.3 |
| A212 | 772.3 |
| A213 | 861.6 |
| A214 | 792.8 |
| A215 | 911.7 |
| A216 | 783.6 |
| A217 | 772.7 |
| A218 | 758.4 |
| A219 | 757.9 |
| A220 | 839.7 |
| A221 | 847.3 |
| A222 | 847.3 |
| A223 | 837 |
| A224 | 772.6 |
| A225 | 757.6 |
| A226 | 800.6 |
| A227 | 727.3 |
| A228 | 870.7 |
| A229 | 810.7 |
| A230 | 839.9 |
| A231 | 867.7 |
| A232 | 773.3 |
| A233 | 727.3 |
| A234 | 727.4 |
| A235 | 852.6 |
| A236 | 792.6 |
| A237 | 852.9 |
| A238 | 852.8 |
| A239 | 850.6 |
| A240 | 906.6 |
| A241 | 797.6 |
| A242 | 829.6 |
| A243 | 898.7 |
| A244 | 912.7 |
| A245 | 756.2 |
| A246 | 769.6 |
| A247 | 811.6 |
| A248 | 866.6 |
| A249 | 866.7 |
| A250 | 866.8 |
| A251 | 891.4 |
| A252 | 825.7 |
| A253 | 825.5 |
| A254 | 825.6 |
| A255 | 825.8 |
| A256 | 770.3 |
| A257 | 883.8 |
| A258 | 854.7 |
| A259 | 853.5 |
| A260 | 890.5 |
| A261 | 873.6 |
| A262 | 873.6 |
| A263 | 837.3 |
| A264 | 881.7 |
| A265 | 865.3 |
| A266 | 865.5 |
| A267 | 865.1 |
| A268 | 813.7 |
| A269 | 867.4 |
| A270 | 772.5 |
| A271 | 913.7 |
| A272 | 899.7 |
| A273 | 867.5 |
| A274 | 851.7 |
| A275 | 811.7 |
| A276 | 891.3 |
| A277 | 907.5 |
| A278 | 897.6 |
| A279 | 843.8 |
| A280 | 856.7 |
| A281 | 857.7 |
| A282 | 965.6 |
| A283 | 965.5 |
| A284 | 805.4 |
| A285 | 768.5 |
| A286 | 850.9 |
| A287 | 909.6 |
| A288 | 925.8 |
| A289 | 855.6 |
| A290 | 855.6 |
| A291 | 755.2 |
| A292 | 755.2 |
| A293 | 811.8 |
| A294 | 793.6 |
| A295 | 384.9 |
| A296 | 770.6 |
| A297 | 899.7 |
| A298 | 906.8 |
| A299 | 905.5 |
| A300 | 865.5 |
| A301 | 869.6 |
| A302 | 869.4 |
| A303 | 785.5 |
| A304 | 935.4 |
| A305 | 901.0 |
| A306 | 901.0 |
| A307 | 757.4 |
| A308 | 741.4 |
| A309 | 771.4 |
| A310 | 804.7 |
| A311 | 889.7 |
| A312 | 910.5 |
| A313 | 752.4 |
| A314 | 887.4 |
| A315 | 887.5 |
| A316 | 869.5 |
| A317 | 869.5 |
| A318 | 839.7 |
| A319 | 839.6 |
| A320 | 783.8 |
| A321 | 841.8 |
| A322 | 785.3 |
| A323 | 799.4 |
| A324 | 979.6 |
| A325 | 755.1 |
| A326 | 755.1 |
| A327 | 855.9 |
| A328 | 853.6 |
| A329 | 763.0 |
| A330 | 763.0 |
| A331 | 855.5 |
| A332 | 854.5 |
| A333 | 885.9 |
| A334 | 813.5 |
| A335 | 870.7 |
| A336 | 785.4 |
| A337 | 769.5 |
| A338 | 809.9 |
| A339 | 799.6 |
| A340 | 739.6 |
| A341 | 743.5 |
| A342 | 852.7 |
| A343 | 852.7 |
| A344 | 820.4 |
| A345 | 820.4 |
| A346 | 732.6 |
| A347 | 732.6 |
| A348 | 730.6 |
| A349 | 730.3 |
| A350 | 747.5 |
| A351 | 745.3 |
| A352 | 745.3 |
| A353 | 833.8 |

The following table of compounds were prepared using the aforementioned methods or variations thereof, as would be known to those of skill in the art, or the methods described below.

TABLE 5
Exemplary Compounds Prepared by Methods of the Present Invention

| Ex# | LCMS (ESI): m/z [M + H] Found |
|---|---|
| A354 | 870.8 |
| A355 | 743.5 |
| A356 | 738.5 |
| A357 | 783.4 |
| A358 | 744.5 |
| A359 | 758.6 |
| A360 | 758.6 |
| A361 | 728.4 |
| A362 | 747.6 |
| A363 | 747.5 |
| A364 | 745.6 |
| A365 | 731.6 |
| A366 | 807.4 |
| A367 | 789.4 |
| A368 | 746.5 |
| A369 | 747.4 |
| A370 | 827.4 |
| A371 | 842.0 |
| A372 | 769.2 |
| A373 | 897.4 |
| A374 | 778.3 |
| A375 | 806.4 |
| A376 | 758.4 |
| A377 | 871 |
| A378 | 760.5 |
| A379 | 760.35 |
| A380 | 760.5 |
| A381 | 784.4 |
| A382 | 758.5 |
| A383 | 733.4 |
| A384 | 825.3 |
| A385 | 741.2 |
| A386 | 741.2 |
| A387 | 807.5 |
| A388 | 840.3 |
| A389 | 910.7 |
| A390 | 910.4 |
| A391 | 809.8 |
| A392 | 768.6 |
| A393 | 768.5 |
| A394 | 770.6 |
| A395 | 770.6 |
| A396 | 900.9 |
| A397 | 886.2 |
| A398 | 774.3 |
| A399 | 744.6 |
| A400 | 743.6 |
| A401 | 918.6 |
| A402 | 825.6 |
| A403 | 803.5 |
| A404 | 803.5 |
| A405 | 826.25 |
| A406 | 856.7 |
| A407 | 745.4 |
| A408 | 881.9 |
| A409 | 753.6 |
| A410 | 911.8 |
| A411 | 885.9 |
| A412 | 760.6 |
| A413 | 784.3 |
| A414 | 741.4 |
| A415 | 769.5 |
| A416 | 829.7 |
| A417 | 820.4 |
| A418 | 866.8 |
| A419 | 755.3 |
| A420 | 755.3 |
| A421 | 856.7 |
| A422 | 739.5 |
| A423 | 740.4 |
| A424 | 741.4 |
| A425 | 763.2 |
| A426 | 941.8 |
| A427 | 941.3 |
| A428 | 910.7 |
| A429 | 899.2 |
| A430 | 742.3 |
| A431 | 756.6 |
| A432 | 756.6 |
| A433 | 898.9 |
| A434 | 885.4 |
| A435 | 746.6 |
| A436 | 757.3 |
| A437 | 872.3 |
| A438 | 927.9 |
| A439 | 820.2 |
| A440 | 828.3 |
| A441 | 828.2 |
| A442 | 866.5 |
| A443 | 763.3 |
| A444 | 738.1 |
| A445 | 770.2 |
| A446 | 756.7 |
| A447 | 742.7 |
| A448 | 813.6 |
| A449 | 910.8 |
| A450 | 976.9 |
| A451 | 840.7 |
| A452 | 856.4 |
| A453 | 838.7 |
| A454 | 838.4 |
| A455 | 799.4 |
| A456 | 813.6 |
| A457 | 827.6 |
| A458 | 771.2 |
| A459 | 958.4 |
| A460 | 928.4 |
| A461 | 894.8 |
| A462 | 813.5 |
| A463 | 820.7 |
| A464 | 771.6 |
| A465 | 771.6 |
| A466 | 757.7 |
| A467 | 856.35 |
| A468 | 771.3 |
| A469 | 756.3 |
| A470 | 745.4 |
| A471 | 754.5 |
| A472 | 742.2 |
| A473 | 785.5 |
| A474 | 799.5 |
| A475 | 925.5 |
| A476 | 925.4 |
| A477 | 870.3 |
| A478 | 840.6 |
| A479 | 837.6 |
| A480 | 837.6 |
| A481 | 932.4 |
| A482 | 932.4 |
| A483 | 936.4 |
| A484 | 952.5 |
| A485 | 784.3 |
| A486 | 757.5 |
| A487 | 771.5 |
| A488 | 757.2 |
| A489 | 757.25 |
| A490 | 800.3 |
| A491 | 790.4 |
| A492 | 790.6 |
| A493 | 858.3 |
| A494 | 850.5 |
| A495 | 850.4 |
| A496 | 756.4 |
| A497 | 910.4 |
| A498 | 790.3 |
| A499 | 790.3 |
| A500 | 911.8 |
| A501 | 911.8 |
| A502 | 952.5 |
| A503 | 936.4 |
| A504 | 835.2 |
| A505 | 835.2 |
| A506 | 837.4 |
| A507 | 837.3 |
| A508 | 854.6 |
| A509 | 868.6 |
| A510 | 883.7 |
| A511 | 898.4 |
| A512 | 897.8 |
| A513 | 897.8 |
| A514 | 787.7 |
| A515 | 883.5 |
| A516 | 883.55 |
| A517 | 844.3 |
| A518 | 828.35 |
| A519 | 770.3 |
| A520 | 1004.3 |
| A521 | 968.9 |
| A522 | 835.5 |
| A523 | 821.3 |
| A524 | 811.5 |
| A525 | 793.4 |
| A526 | 755.2 |
| A527 | 796.6 |
| A528 | 796.3 |
| A529 | 812.5 |
| A530 | 812.3 |
| A531 | 840.2 |
| A532 | 840.2 |
| A533 | 824.2 |
| A534 | 824.2 |
| A535 | 884.3 |
| A536 | 884.3 |
| A537 | 884.25 |
| A538 | 946.3 |
| A539 | 1047.6 |
| A540 | 996.9 |
| A541 | 997.0 |
| A542 | 837.3 |
| A543 | 849.3 |
| A544 | 783.3 |
| A545 | 756.6 |
| A546 | 835.7 |
| A547 | 835.4 |
| A548 | 884.3 |
| A549 | 910.3 |
| A550 | 910.7 |
| A551 | 853.8 |
| A552 | 823.3 |
| A553 | 769.3 |
| A554 | 742.5 |
| A555 | 953.8 |
| A556 | 739.3 |
| A557 | 799.2 |
| A558 | 887.6 |
| A559 | 887.6 |
| A560 | 952.5 |
| A561 | 968.35 |
| A562 | 968.8 |
| A563 | 1011.7 |
| A564 | 995.3 |
| A565 | 1035.7 |
| A566 | 826.5 |
| A567 | 826.6 |
| A568 | 886.7 |
| A569 | 938.8 |
| A570 | 1032.4 |
| A571 | 1032.3 |
| A572 | 828.4 |
| A573 | 824.2 |
| A574 | 834.3 |
| A575 | 875.2 |
| A576 | 824.3 |
| A577 | 959.4 |
| A578 | 959.4 |
| A579 | 868.5 |
| A580 | 868.6 |
| A581 | 879.25 |
| A582 | 879.6 |
| A583 | 866.4 |
| A584 | 896.2 |
| A585 | 833.3 |
| A586 | 887.4 |
| A587 | 887.4 |
| A588 | 894.4 |
| A589 | 812.3 |
| A590 | 812.3 |
| A591 | 1031.7 |
| A592 | 1010.5 |
| A593 | 996.6 |
| A594 | 1010.4 |
| A595 | 868.6 |
| A596 | 858.5 |
| A597 | 865.2 |
| A598 | 918.6 |
| A599 | 868.6 |
| A600 | 868.25 |
| A601 | 872.3 |
| A602 | 879.6 |
| A603 | 884.6 |
| A604 | 884.4 |
| A605 | 884.7 |
| A606 | 880.2 |
| A607 | 896.7 |
| A608 | 904.3 |

### Example A372. Synthesis of (1r,2R,3S)—N-((2²R,6³S,4S)-1¹-ethyl-1²-(2-((S)-1-methoxyethyl) pyridin-3-yl)-22-(methoxymethyl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-5 pyridinacycloundecaphane-4-yl)-2,3-dimethylcyclopropane-1-carboxamide

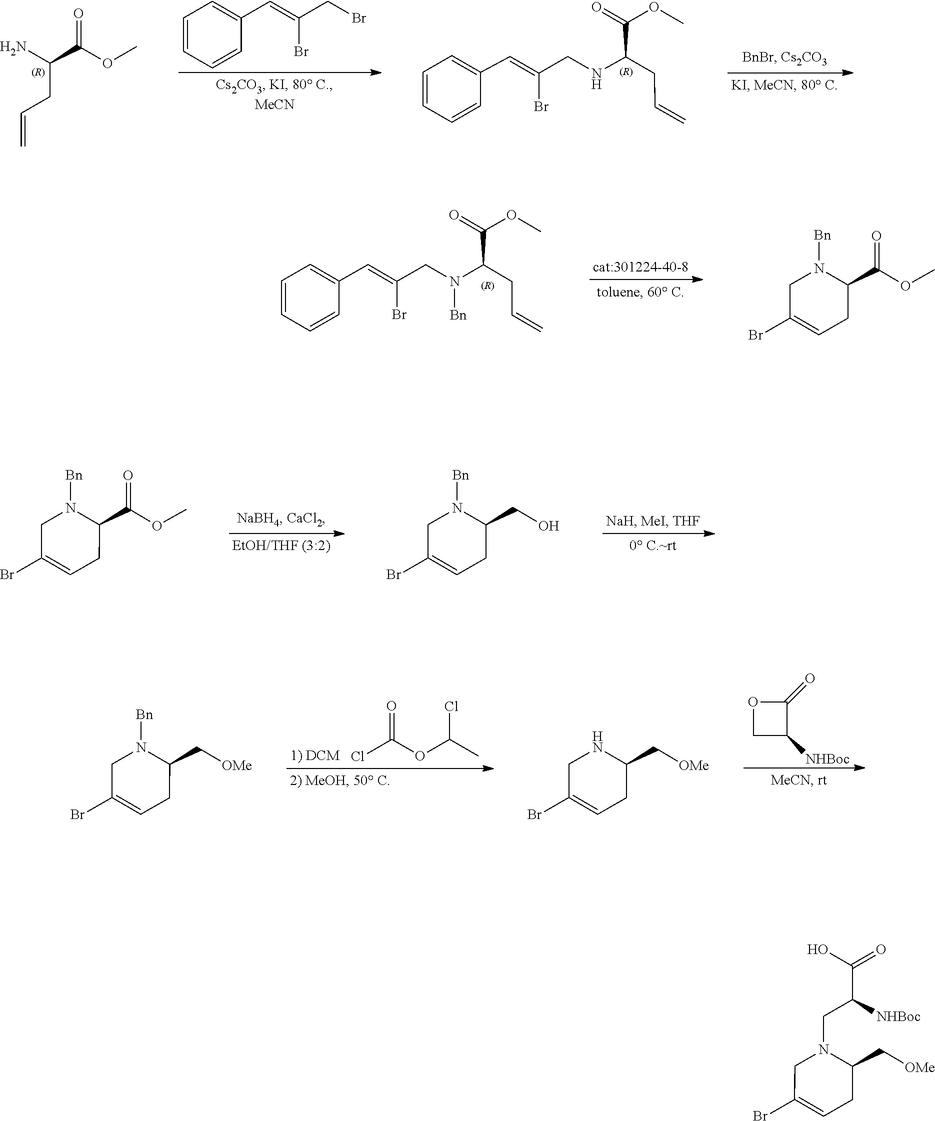

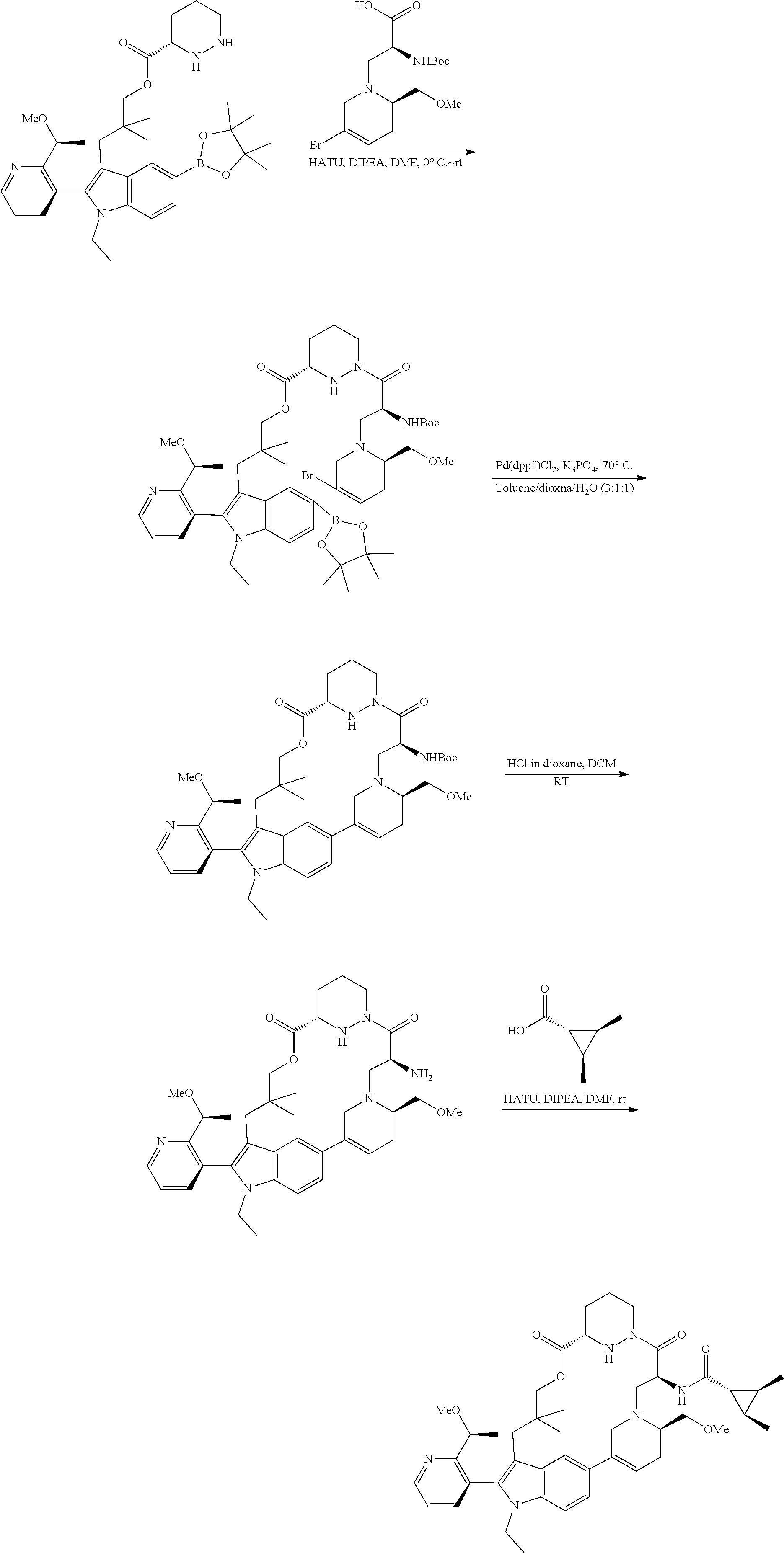

### Step 1.

To a mixture of methyl (R)-2-aminopent-4-enoate (8.0 g, 36.5 mmol) and (Z)-(2,3-dibromoprop-1-en-1-yl)benzene (15.1 g, 54.8 mmol) in MECN (80 mL) under an atmosphere of N₂ was added Cs₂CO₃ (35.7 g, 109.6 mmol) and KI (12.13 g, 73.1 mmol) in portions. The mixture was heated to 80° C. and stirred overnight, then filtered and the filter cake was washed with MeCN (3×20 mL). The filtrate was concentrated under reduced pressure and the residue was purified by silica gel column chromatography to give methyl (R,Z)-2-((2-bromo-3-phenylallyl) amino) pent-4-enoate (12.0 g, 91% yield) as an oil. LCMS (ESI): m/z [M+H]⁺ calc'd for C₁₅H₁₈BrNO₂ 323.1; found 324.1.

### Step 2.

To a mixture of methyl (R,Z)-2-((2-bromo-3-phenylallyl) amino) pent-4-enoate (12.0 g, 37.0 mmol) and BnBr (12.66 g, 74.0 mmol) in MeCN (120 mL) under an atmosphere of N₂ was added Cs₂CO₃ (24.12 g, 74.0 mmol) and KI (6.14 g, 37.0 mmol) in portions. The mixture was stirred at room temperature overnight, then filtered and the filter cake was washed with MeCN (3×20 mL). The filtrate was concentrated under reduced pressure and the residue was purified by silica gel column chromatography to give methyl (R,Z)-2-(benzyl (2-bromo-3-phenylallyl) amino) pent-4-enoate (6.0 g, 35% yield) as an oil. LCMS (ESI): m/z [M+H]⁺ calc'd for C₂₂H₂₄BrNO₂ 415.1; found 416.1 [for 81Br].

### Step 3.

To a mixture of methyl (R,Z)-2-(benzyl (2-bromo-3-phenylallyl) amino) pent-4-enoate (5.8 g, 14.0 mmol) and [1,3-bis (2,4,6-trimethylphenyl) imidazolidin-2-ylidene] dichloro [2-(propan-2-yloxy)phenyl]methylidene] ruthenium (2.63 g, 4.2 mmol) in toluene (580 mL) under an atmosphere of Ar was heated to 60° C. and stirred for 30 min. The mixture was concentrated under reduced pressure and the residue was purified by preparative-HPLC to give methyl (R)-1-benzyl-5-bromo-1,2,3,6-tetrahydropyridine-2-carboxylate (3.7 g, 77% yield) as an oil. LCMS (ESI): m/z [M+H]⁺ calc'd for C₁₄H₁₆BrNO₂ 309.0; found 310.1.

### Step 4.

To a mixture of methyl (R)-1-benzyl-5-bromo-1,2,3,6-tetrahydropyridine-2-carboxylate (3.7 g, 11.9 mmol) and CaCl₂) (2.65 g, 23.9 mmol) in EtOH (22 mL) and THF (15 mL) at 0° C. under an atmosphere of N₂ was added NaBH₄ (1.80 g, 47.7 mmol) in portions. The mixture was warmed to room temperature and stirred for 2 h, then cooled to 0° C., and MeOH and H₂O were added. The mixture was extracted with DCM (2×50 mL), dried over anhydrous Na₂SO₄ and filtered. The filtrate was concentrated under reduced pressure and the residue was purified by preparative HPLC to give (R)-(1-benzyl-5-bromo-1,2,3,6-tetrahydropyridin-2-yl) methanol (2.8 g, 75% yield) as a solid. LCMS (ESI): m/z [M+H]⁺ calc'd for C₁₃H₁₆BrNO 281.0; found 282.3.

### Step 5.

To a mixture of (R)-(1-benzyl-5-bromo-1,2,3,6-tetrahydropyridin-2-yl) methanol (1.0 g, 3.5 mmol) in THF (10 mL) at 0° C. was added NaH, 60% dispersion in oil (0.26 g, 10.6 mmol). The mixture was stirred for 15 min, then Mel (0.75 g, 5.3 mmol) was added and the mixture was allowed to warm to room temperature and stirred for 1 h. The mixture was cooled to 0° C., saturated NH₄Cl was added and the mixture was extracted with EtOAc (2×20 mL). The combined organic layers were dried over anhydrous Na₂SO₄, filtered, the filtrate was concentrated under reduced pressure and the residue was purified by preparative-HPLC to give (R)-1-benzyl-5-bromo-2-(methoxymethyl)-1,2,3,6-tetrahydropyridine (1.0 g, 86% yield) as a solid. LCMS (ESI): m/z [M+H]⁺ calc'd for C₁₄H₁₈BrNO 295.1; found 296.2.

### Step 6.

A mixture of (R)-1-benzyl-5-bromo-2-(methoxymethyl)-1,2,3,6-tetrahydropyridine (1.0 g, 3.4 mmol) and 1-chloroethyl chloroformate (2 mL, 14.0 mmol) in DCM (10 mL) under an atmosphere of N₂ was stirred at room temperature overnight. The mixture was washed with brine (2×10 mL) and the combined aqueous layers were extracted with DCM (10 mL). The combined organic layers were dried over anhydrous Na₂SO₄, filtered and the filtrate was concentrated under reduced pressure. The residue was dissolved in MeOH (10 mL), the mixture was heated to 50° C. under an atmosphere of N₂ and stirred for 2 h, then concentrated under reduced pressure and the residue was purified by preparative-HPLC to give (R)-5-bromo-2-(methoxymethyl)-1,2,3,6-tetrahydropyridine (500 mg, 72% yield) as an oil. LCMS (ESI): m/z [M+H]⁺ calc'd for C₇H₁₂BrNO 205.0; found 206.1.

### Step 7.

To a mixture of (R)-5-bromo-2-(methoxymethyl)-1,2,3,6-tetrahydropyridine (600 mg, 2.9 mmol) and tert-butyl N-[(3S)-2-oxooxetan-3-yl]carbamate (382 mg, 2.0 mmol) in MeCN (6 mL) under an atmosphere of N₂ was stirred overnight. The mixture was concentrated under reduced pressure and the residue was purified by preparative-HPLC to give (S)-3-((R)-5-bromo-2-(methoxymethyl)-3,6-dihydropyridin-1 (2H)-yl)-2-((tert-butoxycarbonyl) amino) propanoic acid (500 mg, 39% yield) as a solid. LCMS (ESI): m/z [M+H]⁺ calc'd for C₁₅H₂₅BrN₂O₅ 392.1; found 393.1.

### Step 8.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-hexahydropyridazine-3-carboxylate (1.25 g, 2.07 mmol) and(S)-3-((R)-5-bromo-2-(methoxymethyl)-3,6-dihydropyridin-1 (2H)-yl)-2-((tert-butoxycarbonyl) amino) propanoic acid (0.49 g, 1.24 mmol) and DIPEA (2.67 g, 20.7 mmol) in DMF (15 mL) under an atmosphere of N₂ was added HATU (0.79 g, 2.07 mmol) in portions. The mixture was warmed to room temperature and stirred for 1 h, then H₂O added and the mixture was extracted with EtOAc (50 mL). The organic layer was washed with brine (3×50 mL), dried over anhydrous Na₂SO₄ and filtered. The filtrate was concentrated under reduced pressure and the residue was purified by silica gel column chromatography to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((S)-3-((R)-5-bromo-2-(methoxymethyl)-3,6-dihydropyridin-1 (2H)-yl)-2-((tert-butoxycarbonyl) amino) propanoyl) hexahydropyridazine-3-carboxylate (880 mg, 39% yield) as a solid. LCMS (ESI): m/z [M+H]⁺ calc'd for C₄₉H₇₂BBrN₆O₉ 980.5; found 981.4 [for 81Br].

### Step 9.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-1-((S)-3-((R)-5-bromo-2-(methoxymethyl)-3,6-dihydropyridin-1 (2H)-yl)-2-((tert-butoxycarbonyl) amino) propanoyl) hexahydropyridazine-3-carboxylate (850 mg, 0.87 mmol) and K₃PO₄ (460 mg, 2.17 mmol) in toluene (4.5 mL), 1,4-dioxane (1.5 mL) and H₂O (1.5 mL) under an atmosphere of N₂ was added Pd(dppf)Cl₂ (64 mg, 0.09 mmol) in portions. The mixture was heated to 70° C. and stirred for 3 h at 70° C., then concentrated under reduced pressure and the residue was purified by silica gel column chromatography to give tert-butyl ((2²R,6³S,4S)-1¹-ethyl-1²-(2-((S)-1-methoxyethyl) pyridin-3-yl)-22-(methoxymethyl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl) carbamate (220 mg, 30% yield) as a solid. LCMS (ESI): m/z [M+H]⁺ calc'd for C₄₃H₆₀N₆O₇ 772.5; found 773.4.

### Step 10.

A mixture of tert-butyl ((2²R,6³S,4S)-1¹-ethyl-1²-(2-((S)-1-methoxyethyl) pyridin-3-yl)-22-(methoxymethyl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl) carbamate (100 mg, 0.13 mmol) and HCl in 1,4-dioxane (2 mL) in DCM (2 mL) was stirred at room temperature for 1 h. The mixture was concentrated under reduced pressure to give (2²R,6³S,4S)-4-amino-1¹-ethyl-1²-(2-((S)-1-methoxyethyl) pyridin-3-yl)-22-(methoxymethyl)-10,10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-5,7-dione (119 mg) as a solid, that was used directly in the next step without further purification.

### Step 11.

To a mixture of (2²R,6³S,4S)-4-amino-1¹-ethyl-1²-(2-((S)-1-methoxyethyl) pyridin-3-yl)-22-(methoxymethyl)-10, 10-dimethyl-21, 22,23,26,61,62,63,64,65,66-decahydro-1¹H-8-oxa-1 (5,3)-indola-6 (1,3)-pyridazina-2 (5,1)-pyridinacycloundecaphane-5,7-dione (20 mg, 0.18 mmol), (1r,2R,3S)-2,3-dimethylcyclopropanecarboxylic acid, and DIPEA (114 mg, 0.89 mmol) in DMF (2 mL) was added HATU (81 mg, 0.21 mmol) in portions. The mixture was stirred at room temperature for 1 h, then purified by preparative-HPLC to give (1r,2R,3S)—N-((2²R,6³S,4S)-1¹-ethyl-1²-(2-((S)-1-methoxyethyl) pyridin-3-yl)-22-(methoxymethyl)-10, 10-dimethyl-5,7-dioxo-21,22,23,26,61,62,63,64,65,66-decahydro-1¹H-8-oxa-1 (5,3)-indola-6 (1,3)-pyridazina-2 (5,1)-pyridinacycloundecaphane-4-yl)-2,3-dimethylcyclopropane-1-carboxamide (24 mg, 18% yield) as a solid. LCMS (ESI): m/z [M+H]⁺ calc'd for C₄₄H₆₀N₆O₆ 768.5; found 769.2; ¹H NMR (400 MHZ, DMSO-d₆) δ 8.90-8.65 (m, 1H), 8.10-7.95 (m, 1H), 7.90-7.75 (m, 1H), 7.57-7.45 (m, 3H), 7.40 (s, 1H), 6.26 (s, 1H), 5.65-5.50 (m, 1H), 5.45-5.30 (m, 1H), 4.39-4.25 (m, 1H), 4.24-4.20 (m, 1H), 4.19-4.10 (m, 1H), 4.09-4.00 (m, 1H), 3.88-3.76 (m, 1H), 3.73-3.62 (m, 2H), 3.62-3.55 (m, 2H), 3.26 (s, 5H), 3.14-3.00 (m, 2H), 2.96-2.87 (m, 1H), 2.79-2.70 (m, 5H), 2.43 (s, 1H), 2.15-2.06 (m, 2H), 1.95-1.86 (m, 1H), 1.86-1.72 (m, 1H), 1.58-1.49 (m, 2H), 1.40-1.30 (m, 3H), 1.10-1.00 (m, 6H), 0.99-0.90 (m, 6H), 0.82 (s, 3H), 0.52 (s, 3H).

### Example A373. Synthesis of (1r,2R,3S)—N-((6³S,4S,Z)-12-(5-(1-(2-(dimethylamino)ethyl)-4-hydroxypiperidin-4-yl)-2-((S)-1-methoxyethyl) pyridin-3-yl)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

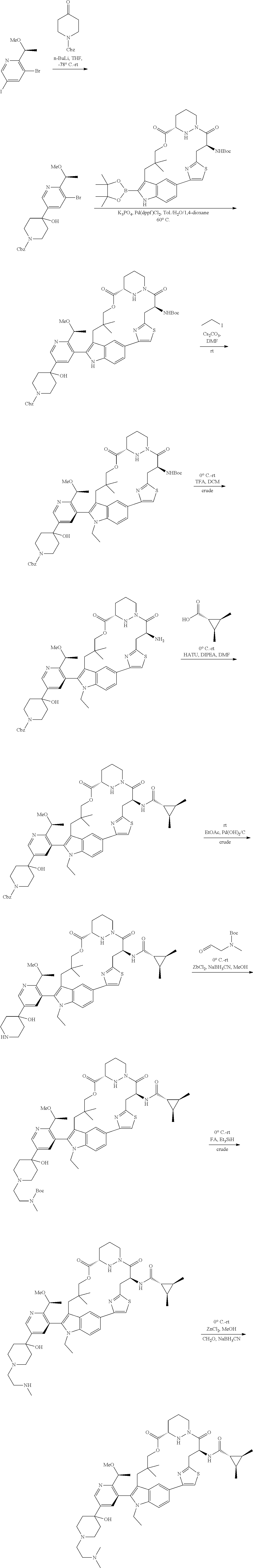

### Step 1.

To a mixture of(S)-3-bromo-5-iodo-2-(1-methoxyethyl) pyridine (5.0 g, 14.6 mmol) in THF (40 mL) at −78° C. under an atmosphere of N₂ was added n-BuLi in hexanes (5.85 mL, 14.6 mmol) dropwise. The mixture was stirred at −78° C. for 1 h, then benzyl 4-oxopiperidine-1-carboxylate (6.82 g, 29.2 mmol) was added. The mixture was allowed to warm to 0° C. Saturated NH₄Cl (3 mL) was added, the mixture was diluted with H₂O (20 mL) and extracted with EtOAc (3×20 mL). The combined organic layers were dried over anhydrous Na₂SO₄, filtered, the filtrate was concentrated under reduced pressure and the residue was purified by silica gel column chromatography to give benzyl(S) -4-(5-bromo-6-(1-methoxyethyl) pyridin-3-yl)-4-hydroxypiperidine-1-carboxylate (800 mg, 12% yield) as a solid. LCMS (ESI): m/z [M+H]⁺ calc'd for C₂₁H₂₅BrN₂O₄ 448.1; found 449.2.

### Step 2.

To a mixture of tert-butyl ((6³S,4S,Z)-10, 10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl) carbamate (500 mg, 0.72 mmol) and benzyl(S)-4-(5-bromo-6-(1-methoxyethyl) pyridin-3-yl)-4-hydroxypiperidine-1-carboxylate (648 mg, 1.4 mmol) in toluene (9 mL), H₂O (3 mL) and 1,4-dioxane (3 mL) under an atmosphere of N₂ was added K₃PO₄ (459 mg, 2.16 mmol) and Pd(dppf)Cl₂·CH₂Cl₂ (59 mg, 0.07 mmol) in portions. The mixture was heated to 60° C. and stirred for 4 h, then diluted with H₂O (10 mL) and extracted with EtOAc (3×10 mL). The combined organic layers were dried over anhydrous Na₂SO₄, filtered, the filtrate was concentrated under reduced pressure and the residue was purified by silica gel column chromatography to give benzyl 4-(5-((6³S,4S,Z)-4-((tert-butoxycarbonyl) amino)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (1.16 g, 75% yield) as a solid. LCMS (ESI): m/z [M+H]⁺ calc'd for C₅₀H₆₁N₇O₉S 935.4; found 936.4.

### Step 3.

To a mixture of benzyl 4-(5-((6³S,4S,Z)-4-((tert-butoxycarbonyl) amino)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (1.1 g, 1.18 mmol) and Cs₂CO₃ (1.91 g, 5.88 mmol) in DMF (15 mL) at 0° C. was added iodoethane (0.64 g, 4.1 mmol) dropwise. The mixture was warmed to room temperature and stirred for 2 h, then diluted with H₂O (15 mL) and extracted with EtOAc (3×20 mL). The combined organic layers were dried over anhydrous Na₂SO₄, filtered, the filtrate was concentrated under reduced pressure and the residue was purified by preparative-TLC to give benzyl 4-(5-((6³S,4S,Z)-4-((tert-butoxycarbonyl) amino)-1¹-ethyl-10, 10-dimethyl-5,7-dioxo-61, 62,63,64,65,66-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (110 mg, 9% yield) as a solid. LCMS (ESI): m/z [M+H]⁺ calc'd for C₅₂H₆₅N₇O₉S 963.5; found 964.4.

### Step 4.

To a mixture of benzyl 4-(5-((6³S,4S,Z)-4-((tert-butoxycarbonyl) amino)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl)-4-hydroxypiperidine-1-carboxylate (110 mg, 0.11 mmol) in DCM (3 mL) at 0° C. was added TFA (1 mL) dropwise. The mixture was warmed to room temperature and stirred for 1.5 h, then concentrated under reduced pressure to give benzyl 4-(5-((6³S,4S,Z)-4-amino-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (120 mg) as a solid. LCMS (ESI): m/z [M+H]⁺ calc'd for C₄₇H₅₇N₇O₇S 863.4; found 864.5.

### Step 5.

To a mixture of benzyl 4-(5-((6³S,4S,Z)-4-amino-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (120 mg, 0.14 mmol) and (1R,2R,3S)-2,3-dimethylcyclopropane-1-carboxylic acid (32 mg, 0.28 mmol) in DMF (4 mL) at 0° C. was added DIPEA (180 mg, 1.39 mmol) and HATU (158 mg, 0.42 mmol) in portions. The mixture was warmed to room temperature and stirred for 2 h, then diluted with H₂O (10 mL) and extracted with EtOAc (3×10 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, MECN in water (0.05% TFA), 0% to 100% gradient in 300 min; detector, UV 254 nm. To afford benzyl 4-(5-((6³S,4S,Z)-4-((1r,2R,3S)-2,3-dimethylcyclopropane-1-carboxamido)-11-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (120 mg, 89% yield) as a brown solid. LCMS (ESI): m/z [M+H]⁺ calc'd for C₅₃H₆₅N₇O₈S 959.4; found 960.4.

### Step 6.

A mixture of benzyl 4-(5-((6³S,4S,Z)-4-((1r,2R,3S)-2,3-dimethylcyclopropane-1-carboxamido)-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidine-1-carboxylate (120 mg, 0.13 mmol) and Pd (OH) 2, 30% on carbon (80 mg, 0.25 mmol) in EtOAc (2 mL) was stirred under an atmosphere of H₂ overnight. The mixture was filtered, the filter cake was washed with MeOH (3×8 mL) and the filtrate was concentrated under reduced pressure to give (1r,2R,3S)—N-((6³S,4S,Z)-1¹-ethyl-1²-(5-(4-hydroxypiperidin-4-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (45 mg) as an oil. LCMS (ESI): m/z [M+H]⁺ calc'd for C₄₅H₅₉N₇O₆S 825.4; found 826.4.

### Step 7.

To a mixture of (1r,2R,3S)—N-((6³S,4S,Z)-1¹-ethyl-1²-(5-(4-hydroxypiperidin-4-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (50 mg, 0.06 mmol) and tert-butyl N-methyl-N-(2-oxoethyl) carbamate (52 mg, 0.31 mmol) in MeOH (2 mL) at 0° C. was added ZnCl₂ (83 mg, 0.61 mmol) in portions. The mixture was warmed to room temperature and stirred for 30 min, then cooled to 0° C. and NaBH₃CN (11 mg, 0.18 mmol) added in portions. The mixture was warmed to room temperature and stirred for 2 h, then the residue was purified by preparative-HPLC to give tert-butyl (2-(4-(5-((6³S,4S,Z)-4-((1r,2R,3S)-2,3-dimethylcyclopropane-1-carboxamido)-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-4-hydroxypiperidin-1-yl)ethyl) (methyl) carbamate (30 mg, 50% yield) as a yellow oil. LCMS (ESI): m/z [M+H]⁺ calc'd for C₅₃H₇₄N₈O₈S 982.5; found 983.6.

### Step 8.

To a mixture of tert-butyl (2-(4-(5-((6³S,4S,Z)-4-((1r,2R,3S)-2,3-dimethylcyclopropane-1-carboxamido)-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-1²-yl)-6-((S)-1-methoxyethyl) pyridin-3-yl)-4-hydroxypiperidin-1-yl)ethyl) (methyl) carbamate (30 mg, 0.03 mmol) in FA (1.5 mL) at 0° C. was added Et₃SiH (18 mg, 0.16 mmol). The mixture was warmed to room temperature and stirred for 40 min, then concentrated under reduced pressure to give (1r,2R,3S)—N-((6³S,4S,Z)-1¹-ethyl-1²-(5-(4-hydroxy-1-(2-(methylamino)ethyl) piperidin-4-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (25 mg) as a solid, which was used directly in the next step without further purification. LCMS (ESI): m/z [M+H]⁺ calc'd for C₄₈H₆₆N₈O₆S 882.5; found 883.6.

### Step 9.

To a mixture of (1r,2R,3S)—N-((6³S,4S,Z)-1¹-ethyl-1²-(5-(4-hydroxy-1-(2-(methylamino)ethyl) piperidin-4-yl)-2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (30 mg, 0.03 mmol) and formaldehyde (31 mg, 1.0 mmol) in MeOH (3 mL) at 0° C. was added ZnCl₂ (46 mg, 0.34 mmol) in portions. The mixture was warmed to room temperature and stirred for 30 min, then cooled to 0° C. and NaBH₃CN (6.4 mg, 0.10 mmol) was added in portions. The mixture was warmed to room temperature and stirred for 2 h, then purified by preparative-HPLC to give (1r,2R,3S)—N-((6³S,4S,Z)-12-(5-(1-(2-(dimethylamino)ethyl)-4-hydroxypiperidin-4-yl)-2-((S)-1-methoxyethyl) pyridin-3-yl)-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (0.7 mg, 2% yield) as a solid. LCMS (ESI): m/z [M+H]⁺ calc'd for C₄₉H₆₈N₈O₆S 896.5; found 897.4; ¹H NMR (400 MHz, DMSO-d₆) δ 8.83 (d, J=2.3 Hz, 1H), 8.49 (d, J=1.6 Hz, 1H), 8.38 (d, J=9.1 Hz, 2H), 7.81 (s, 1H), 7.78-7.68 (m, 2H), 7.57 (d, J=8.7 Hz, 1H), 5.57 (t, J=9.3 Hz, 1H), 5.06 (d, J=12.1 Hz, 1H), 4.36-4.07 (m, 6H), 3.56 (s, 2H), 3.24 (s, 3H), 3.18-3.11 (m, 1H), 2.93 (d, J=14.5 Hz, 1H), 2.75-2.60 (m, 2H), 2.43 (d, J=11.4 Hz, 4H), 2.37 (s, 2H), 2.15 (s, 7H), 2.02 (d, J=16.7 Hz, 4H), 1.78 (s, 2H), 1.66 (d, J=12.6 Hz, 2H), 1.50 (s, 1H), 1.36 (d, J=6.1 Hz, 3H), 1.24 (s, 1H), 1.16 (s, 2H), 1.15-0.93 (m, 6H), 0.92-0.63 (m, 6H), 0.31 (s, 3H).

### Example A387. Synthesis of (1r,2R,3S)—N-((6³S,3S,4S,Z)-3-(2,2-difluoroethoxy)-11-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

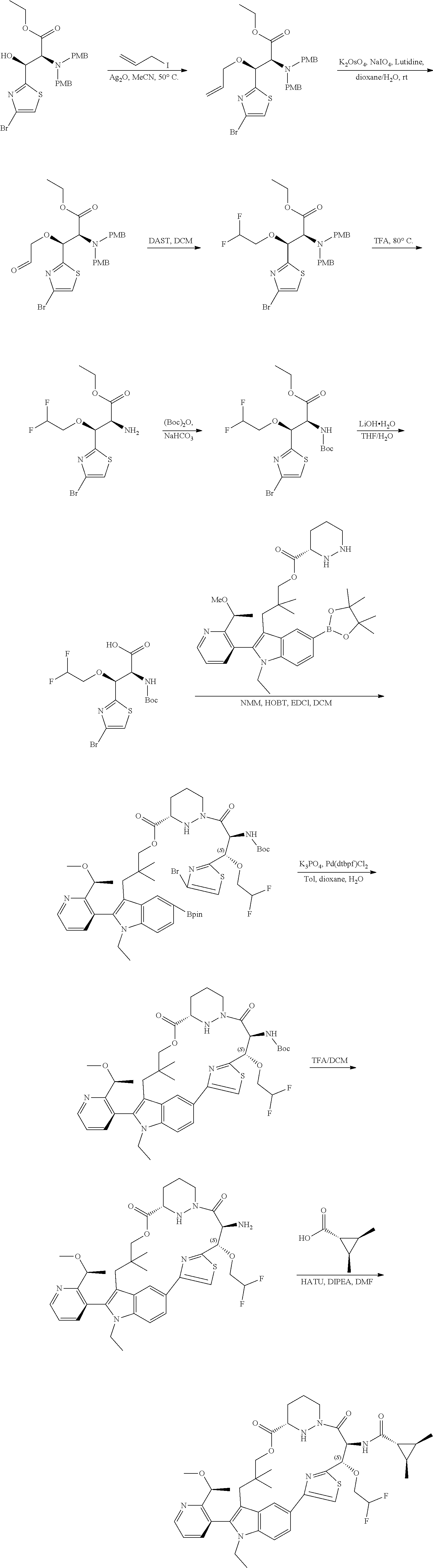

### Step 1.

To a mixture of ethyl (2S,3S)-2-(bis (4-methoxybenzyl) amino)-3-(4-bromothiazol-2-yl)-3-hydroxypropanoate (1.0 g, 1.9 mmol) in MeCN (10 mL) at room temperature under an atmosphere of N₂ was added Ag₂O (2.17 g, 9.4 mmol) and allyl iodide (1.57 g, 9.36 mmol). The resulting mixture was heated to 60° C. and stirred for 16 h, then filtered, and the filter cake was washed with EtOAc (3×20 mL). The filtrate was concentrated under reduced pressure and the residue was purified by silica gel column chromatography to give ethyl (2S,3S)-3-(allyloxy)-2-(bis (4-methoxybenzyl) amino)-3-(4-bromothiazol-2-yl) propanoate (1.0 g, 93% yield) as a solid. LCMS (ESI): m/z [M+H]⁺ calc'd for C₂₇H₃₁BrN₂O₅S 574.1 & 576.1; found 575.1 & 577.1.

### Step 2.

To a mixture of ethyl (2S,3S)-3-(allyloxy)-2-(bis (4-methoxybenzyl) amino)-3-(4-bromothiazol-2-yl) propanoate (1.0 g, 1.7 mmol) in 1,4-dioxane (10 mL) and H₂O (10 mL) at 0° C. was added 2,6-lutidine (0.37 g, 3.48 mmol) and K₂OsO₄·2H₂O (0.03 g, 0.09 mmol). The mixture was stirred at 0° C. for 15 min then NaIO₄ (1.49 g, 6.95 mmol) was added in portions. The mixture was warmed to room temperature and stirred for 2.5 h, then diluted with H₂O (50 mL) and extracted with EtOAc (3×50 mL). The combined organic layers were washed with brine (2×50 mL), dried over anhydrous Na₂SO₄, filtered and the filtrate was concentrated under reduced pressure to give ethyl (2S,3S)-2-(bis (4-methoxybenzyl) amino)-3-(4-bromothiazol-2-yl)-3-(2-oxoethoxy) propanoate (1.2 g) as an oil, which was used directly in the next step without further purification. LCMS (ESI): m/z [M+H]⁺ calc'd for C₂₆H₂₉BrN₂O₆S 576.1 & 578.1; found 577.4 & 579.4.

### Step 3.

To a mixture of ethyl (2S,3S)-2-(bis (4-methoxybenzyl) amino)-3-(4-bromothiazol-2-yl)-3-(2-oxoethoxy) propanoate (1.2 g, 2.1 mmol) in DCM (20 mL) at −15° C. under an atmosphere of N₂ was added DAST (0.37 g, 2.3 mmol) dropwise. The mixture was warmed to room temperature and stirred for 1.5 h, then re-cooled to 0° C. and further DAST (0.37 g, 2.3 mmol) added dropwise. The mixture was warmed to room temperature and stirred for 1 h, then cooled to 0° C. and saturated NH₄Cl (2 mL) added. The mixture was diluted with H₂O (20 mL) and extracted with DCM (3×20 mL). The combined organic layers were washed with brine (2×50 mL), dried over anhydrous Na₂SO₄ and filtered. The filtrate was concentrated under reduced pressure and the residue was purified by preparative-TLC to give ethyl (2S,3S)-2-(bis (4-methoxybenzyl) amino)-3-(4-bromothiazol-2-yl)-3-(2,2-difluoroethoxy) propanoate (270 mg, 22% yield) as an oil. LCMS (ESI): m/z [M+H]⁺ calc'd for C₂₆H₂₉BrF₂N₂O₅S 598.1 & 600.1; found 599.1 & 601.1.

### Step 4.

A mixture of ethyl (2S,3S)-2-(bis (4-methoxybenzyl) amino)-3-(4-bromothiazol-2-yl)-3-(2,2-difluoroethoxy) propanoate (240 mg, 0.40 mmol) in TFA (3 mL) was heated to 80° C. and stirred for 8 h, then concentrated under reduced pressure to give ethyl (2S,3S)-2-amino-3-(4-bromothiazol-2-yl)-3-(2,2-difluoroethoxy) propanoate (245 mg) as an oil, that was used directly in the next step without further purification. LCMS (ESI): m/z [M+H]⁺ calc'd for C₁₀H₁₃BrF₂N₂O₃S 358.0 & 360.0; found 359.0 & 361.0.

### Step 5.

To a mixture of (2S,3S)-2-amino-3-(4-bromothiazol-2-yl)-3-(2,2-ethyl difluoroethoxy) propanoate (230 mg, 0.64 mmol) and NaHCO₃ (108 mg, 1.29 mmol) in H₂O (0.9 mL) and THF (3 mL) was added (Boc) 20 (147 mg, 0.67 mmol). The mixture was stirred at room temperature overnight, then diluted with H₂O (10 mL) and extracted with EtOAc (3×10 mL). The combined organic layers were washed with brine (3×20 mL), dried over anhydrous Na₂SO₄ and filtered. The filtrate was concentrated under reduced pressure and the residue was purified by preparative-TLC to give ethyl (2S,3S)-3-(4-bromothiazol-2-yl)-2-((tert-butoxycarbonyl) amino)-3-(2,2-difluoroethoxy) propanoate (145 mg, 49% yield) as a solid. LCMS (ESI): m/z [M+H]⁺ calc'd for C₁₅H₂₁BrF₂N₂O₅S 458.0 & 460.0; found 459.0 & 461.0.

### Step 6.

To a mixture of ethyl (2S,3S)-3-(4-bromothiazol-2-yl)-2-((tert-butoxycarbonyl) amino)-3-(2,2-difluoroethoxy) propanoate (140 mg, 0.31 mmol) in THF (1.5 mL) at 0° C. was added LiOH·H₂O (64 mg, 1.53 mmol) in H₂O (1.5 mL) dropwise. The mixture was warmed to room temperature and stirred for 1 h, then acidified to PH ˜5 with aqueous HCl, then extracted with EtOAc (3×10 mL). The combined organic layers were washed with brine (3×20 mL), dried over anhydrous Na₂SO₄, filtered and the filtrate was concentrated under reduced pressure to give (2S,3S)-3-(4-bromothiazol-2-yl)-2-((tert-butoxycarbonyl) amino)-3-(2,2-difluoroethoxy) propanoic acid (100 mg) as an oil. LCMS (ESI): m/z [M+H]⁺ calc'd for C₁₃H₁₇BrF₂N₂O₅S 430.0 & 432.0; found 431.0 & 433.0.

### Step 7.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-hexahydropyridazine-3-carboxylate (134 mg, 0.22 mmol) and NMM (335 mg, 3.32 mmol) in DCM (6 mL) at 0° C. under an atmosphere of N₂ was added (2S,3S)-3-(4-bromothiazol-2-yl)-2-((tert-butoxycarbonyl) amino)-3-(2,2-difluoroethoxy) propanoic acid (95 mg, 0.22 mmol) and HOBT (6 mg, 0.04 mmol) and EDCI (85 mg, 0.44 mmol). The mixture was warmed to room temperature and stirred overnight, then diluted with H₂O (10 mL) and extracted with DCM (3×10 mL). The combined organic layers were washed with brine (3×20 mL), dried over anhydrous Na₂SO₄ and filtered. The filtrate was concentrated under reduced pressure and the residue was purified by preparative-TLC to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((2S,3S)-3-(4-bromothiazol-2-yl)-2-((tert-butoxycarbonyl) amino)-3-(2,2-difluoroethoxy) propanoyl) hexahydropyridazine-3-carboxylate (70 mg, 36% yield) as a solid. LCMS (ESI): m/z [M+H]⁺ calc'd for C₄₇H₆₄BBrF₂N₆O₉S 1016.4 & 1018.4; found 1017.3 & 1019.4.

### Step 8.

To a mixture of mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((25,3S)-3-(4-bromothiazol-2-yl)-2-((tert-butoxycarbonyl) amino)-3-(2,2-difluoroethoxy) propanoyl) hexahydropyridazine-3-carboxylate (50 mg, 0.05 mmol) and K₃PO₄ (31 mg, 0.15 mmol) in toluene (3 mL), 1,4-dioxane (1 mL) and H₂O (1 mL) under an atmosphere of N₂ was added Pd(DtBPF)Cl₂ (13 mg, 0.02 mmol) in portions. The mixture was heated to 60° C. and stirred for 1 h, then was extracted with EtOAc (3×10 mL). The combined organic layers were washed with brine (3×20 mL), dried over anhydrous Na₂SO₄ and filtered. The filtrate was concentrated under reduced pressure and the residue was purified by preparative-TLC to give tert-butyl ((6³S,3S,4S,Z)-3-(2,2-difluoroethoxy)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (27 mg, 59% yield) as a solid. LCMS (ESI): m/z [M+H]⁺ calc'd for C₄₁H₅₂F₂N₆O₇S 810.4; found 811.4.

### Step 9.

To a mixture tert-butyl ((6³S,3S,4S,Z)-3-(2,2-difluoroethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (25 mg, 0.02 mmol) in DCM (1.5 mL) at 0° C. was added TFA (0.5 mL) dropwise. The mixture was warmed to room temperature and stirred for 1 h, then concentrated under reduced pressure to give (6³S,3S,4S,Z)-4-amino-3-(2,2-difluoroethoxy)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (25 mg) as a solid, which was used directly in the next step without further purification. LCMS (ESI): m/z [M+H]⁺ calc'd for C₃₆H₄₄F₂N₆O₅S 710.3; found 711.3.

### Step 10.

To a mixture of (6³S,3S,4S,Z)-4-amino-3-(2,2-difluoroethoxy)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (25 mg, 0.04 mmol) and (1r,2R,3S)-2,3-dimethylcyclopropane-1-carboxylic acid (6 mg, 0.05 mmol) in DMF (1 mL) at 0° C. under an atmosphere of N₂ was added DIPEA (45 mg, 0.35 mmol) and HATU (27 mg, 0.07 mmol) in portions. The mixture was warmed to room temperature and stirred for 2 h, then H₂O (10 mL) added and the mixture was extracted with EtOAc (3×10 mL). The combined organic layers were washed with brine (3×20 mL), dried over anhydrous Na₂SO₄ and filtered. The filtrate was concentrated under reduced pressure and the residue was purified by preparative-HPLC to give (1r,2R,3S)—N-((6³S,3S,4S,Z)-3-(2,2-difluoroethoxy)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (3.3 mg, 12% yield) as a solid. LCMS (ESI): m/z [M+H]⁺ calc'd for C₄₂H₅₂F₂N₆O₆S 806.4; found 807.2; ¹H NMR (400 MHZ, DMSO-d₆) δ 8.95-8.70 (m, 1H), 8.50 (s, 1H), 8.13 (s, 1H), 7.95 (s, 1H), 7.88-7.79 (m, 2H), 7.76 (d, J=8.6 Hz, 1H), 7.59 (d, J=8.7 Hz, 1H), 7.54 (dd, J=7.7, 4.8 Hz, 1H), 7.27-6.88 (m, 1H), 6.36-6.04 (m, 1H), 5.97 (d, J=9.9 Hz, 1H), 5.20 (d, J=12.8 Hz, 2H), 4.25 (dd, J=20.3, 10.5 Hz, 4H), 4.14-3.92 (m, 2H), 3.92-3.73 (m, 1H), 3.59 (q, J=10.9 Hz, 2H), 3.20 (s, 3H), 2.81 (d, J=13.7 Hz, 2H), 2.12-1.93 (m, 1H), 1.78 (d, J=23.7 Hz, 2H), 1.59-1.45 (m, 2H), 1.38 (d, J=6.1 Hz, 3H), 1.21 (d, J=20.3 Hz, 1H), 1.16-1.01 (m, 7H), 0.95-0.73 (m, 6H), 0.43 (s, 3H).

### Example A389. Synthesis of (1r,2R,3S)—N-((6³S,3S,4S,Z)-3-ethoxy-1¹-ethyl-1²-(2-((S)-1-methoxyethyl)-5-((8S,9aS)-octahydropyrido [2,1-c][1,4]oxazin-8-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2,3-dimethylcyclopropane-1-carboxamide

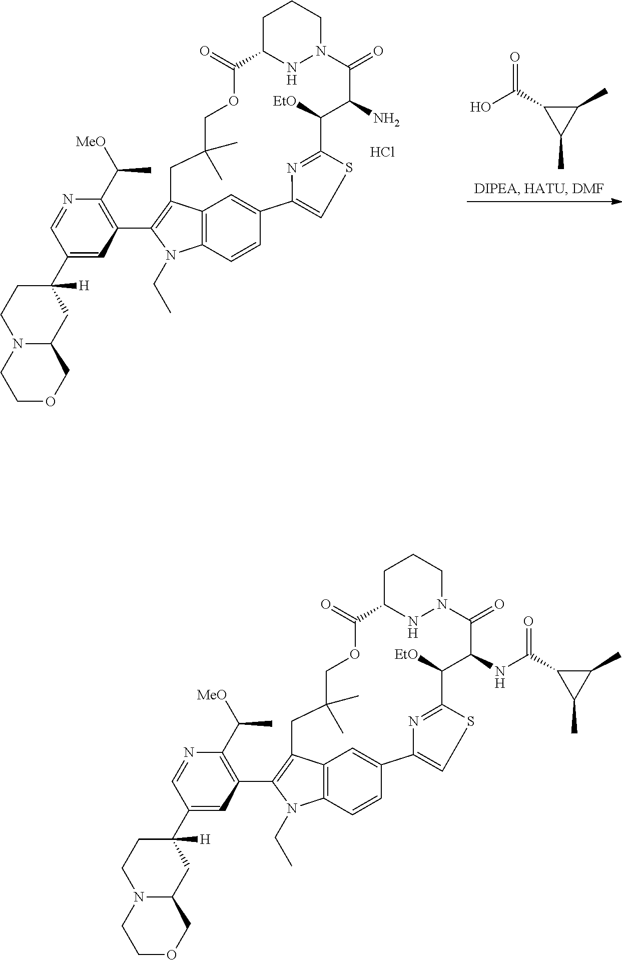

### Step 1.

To a mixture of (6³S,3S,4S,Z)-4-amino-3-ethoxy-1¹-ethyl-1²-(2-((S)-1-methoxyethyl)-5-((8S,9aS)-octahydropyrido [2,1-c][1,4]oxazin-8-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione HCl salt (90 mg, 0.11 mmol) and (1R,2R,3S)-2,3-dimethylcyclopropane-1-carboxylic acid (19 mg, 0.17 mmol) in DMF (3 mL) at 0° C. was added DIPEA (429 mg, 3.3 mmol) and HATU (63 mg, 0.17 mmol) in portions. The mixture was warmed to room temperature and stirred for 2 h, then diluted with H₂O (10 mL) and the mixture was extracted with EtOAc (2×10 mL). The combined organic layers were washed with brine (3×10 mL), then concentrated under reduced pressure and the residue was purified by preparative-HPLC to give (1r,2R,3S)—N-((6³S,3S,4S,Z)-3-ethoxy-1¹-ethyl-1²-(2-((S)-1-methoxyethyl)-5-((8S,9aS)-octahydropyrido [2,1-c][1,4]oxazin-8-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2,3-dimethylcyclopropane-1-carboxamide (13.8 mg, 13% yield) as a solid. LCMS (ESI): m/z [M+H]⁺ calc'd for C₅₀H₆₇N₇O₇S 909.5; found 910.7; ¹H NMR (400 MHZ, DMSO-d₆) δ 8.77 (s, 1H), 8.50 (s, 1H), 7.93 (s, 1H), 7.83-7.51 (m, 4H), 5.96-5.77 (m, 1H), 5.17 (d, J=11.5 Hz, 1H), 4.92 (s, 1H), 4.45-4.03 (m, 5H), 3.57 (ddd, J=34.6, 24.2, 13.9 Hz, 8H), 3.23 (d, J=8.3 Hz, 4H), 3.08 (d, J=10.4 Hz, 2H), 2.84 (d, J=45.9 Hz, 3H), 2.67 (s, 2H), 2.23 (s, 2H), 2.17-2.01 (m, 7H), 1.81 (s, 3H), 1.53 (s, 4H), 1.45-1.30 (m, 4H), 1.24 (s, 1H), 1.16 (td, J=7.0, 1.9 Hz, 4H), 1.06 (dd, J=12.1, 5.1 Hz, 6H), 0.97-0.76 (m, 8H), 0.38 (s, 3H).

### Example A390. Synthesis of (1r,2R,3S)—N-((6³S,3S,4S,Z)-3-ethoxy-1¹-ethyl-1²-(2-((S)-1-methoxyethyl)-5-((8R,9aS)-octahydropyrido [2,1-c][1,4]oxazin-8-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2,3-dimethylcyclopropane-1-carboxamide

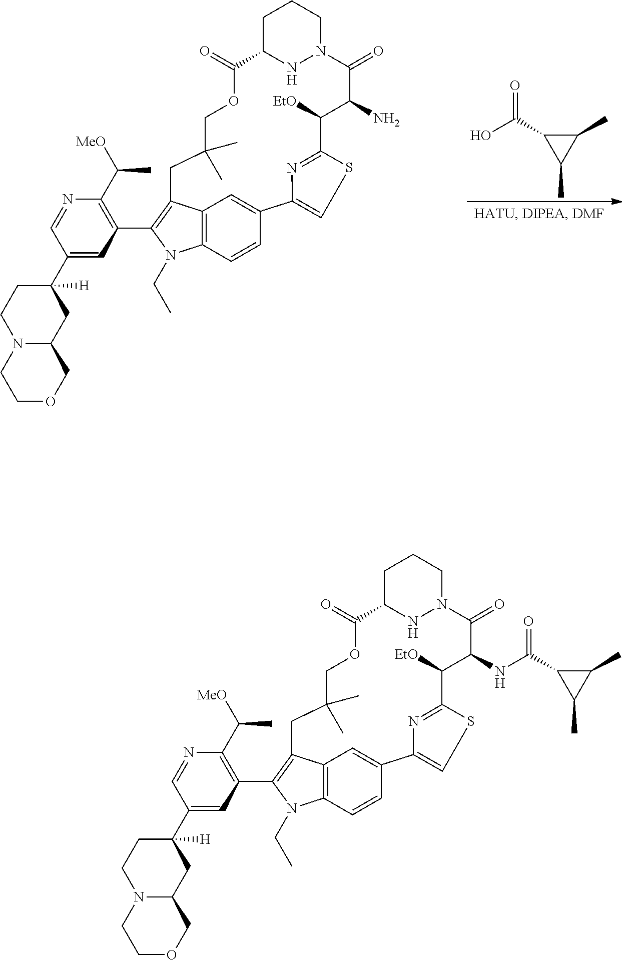

### Step 1.

To a mixture of (6³S,3S,4S,Z)-4-amino-3-ethoxy-1¹-ethyl-1²-(2-((S)-1-methoxyethyl)-5-((8R,9aS)-octahydropyrido [2,1-c][1,4]oxazin-8-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (100 mg, 0.12 mmol) and (1R,2R,3S)-2,3-dimethylcyclopropane-1-carboxylic acid (21 mg, 0.18 mmol) in DMF (3 mL) at −10° C. under an atmosphere of N₂ was added DIPEA (476 mg, 3.7 mmol) and HATU (56 mg, 0.15 mmol, 1.2 equiv) in portions. The mixture was stirred at −10° C. for 1.5 h, then diluted with brine (5 mL) and extracted with EtOAc (3×10 mL). The combined organic layers were washed with brine (3×10 mL), dried over anhydrous Na₂SO₄ and the filtered. The filtrate was concentrated under reduced pressure and the residue was purified by preparative-HPLC to give (1r,2R,3S)—N-((6³S,3S,4S,Z)-3-ethoxy-1¹-ethyl-1²-(2-((S)-1-methoxyethyl)-5-((8R,9aS)-octahydropyrido [2,1-c][1,4]oxazin-8-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (22 mg, 20% yield) as a solid. LCMS (ESI): m/z [M+H]⁺ calc'd for C₅₀H₆₇N₇O₇S 909.5; found 910.7; ¹H NMR (400 MHZ, DMSO-d₆) δ 8.65 (d, J=2.1 Hz, 1H), 8.49 (s, 1H), 7.92 (s, 1H), 7.74 (d, J=8.5 Hz, 1H), 7.68-7.54 (m, 3H), 5.88 (d, J=9.8 Hz, 1H), 5.17 (d, J=12.1 Hz, 1H), 4.93 (s, 1H), 4.36-3.99 (m, 5H), 3.80-3.43 (m, 7H), 3.24-3.05 (m, 4H), 2.92-2.72 (m, 4H), 2.70-2.59 (m, 1H), 2.28-1.99 (m, 5H), 1.89-1.59 (m, 6H), 1.52 (q, J=8.2, 6.3 Hz, 2H), 1.42-1.22 (m, 5H), 1.20-0.99 (m, 12H), 0.86 (d, J=25.0 Hz, 7H), 0.39 (s, 3H).

### Example A391. Synthesis of (1r,2R,3S)—N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-2,3-dimethylcyclopropane-1-carboxamide

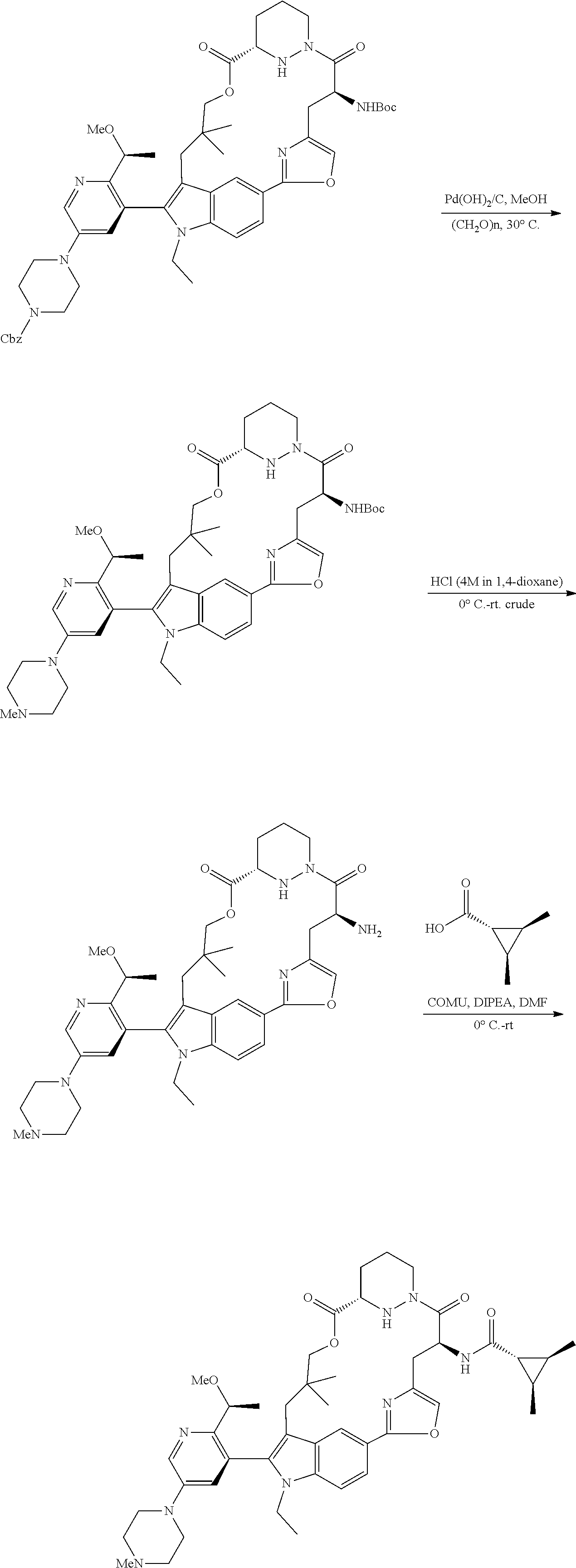

### Step 1.

A mixture of benzyl 4-(5-((6³S,4S,Z)-4-((tert-butoxycarbonyl) amino)-1¹-ethyl-10, 10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-1²-yl)-6-((S)-1-methoxyethyl) pyridin-3-yl) piperazine-1-carboxylate (150 mg, 0.16 mmol), paraformaldehyde (36 mg, 0.81 mmol) and Pd (OH) 2, 30% weight on carbon (151 mg, 0.32 mmol) in MeOH (3 mL) was hydrogenated at 30° C. for 2 h. The mixture was filtered through a pad of Celite and the filtrate was concentrated under reduced pressure to give tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl) carbamate (134 mg) as a solid. LCMS (ESI): m/z [M+H]⁺ calc'd for C₄₄H₆₀N₈O₇ 812.5; found 813.4.

### Step 2.

To mixture a of tert-butyl ((6³S,4S,Z)-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl) carbamate (134 mg, 0.17 mmol) in DCM (1.5 mL) at 0° C. was added TFA (1.50 mL) in portions. The mixture was warmed to room temperature and stirred for 2 h, then concentrated under reduced pressure to give (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (2,4)-oxazola-1 (5,3)-indola-6 (1,3)-pyridazinacycloundecaphane-5,7-dione (318 mg) as an oil. LCMS (ESI): m/z [M+H]⁺ calc'd for C₃₉H₅₂N₈O₅ 712.4; found 713.4.

### Step 3.

To a mixture of (6³S,4S,Z)-4-amino-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (2,4)-oxazola-1 (5,3)-indola-6 (1,3)-pyridazinacycloundecaphane-5,7-dione (130 mg, 0.18 mmol), (1R,2R,3S)-2,3-dimethylcyclopropane-1-carboxylic acid (42 mg, 0.36 mmol) and DIPEA (236 mg, 1.8 mmol) in DMF (2 mL) at 0° C. was added COMU (117 mg, 0.27 mmol) in portions. The mixture was warmed to room temperature and stirred for 2 h, then the residue was purified by preparative-HPLC to give (1r,2R,3S)—N-((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (2,4)-oxazola-1 (5,3)-indola-6 (1,3)-pyridazinacycloundecaphane-4-yl)-2,3-dimethylcyclopropane-1-carboxamide (28 mg, 19% yield) as a solid. LCMS (ESI): m/z [M+H]⁺ calc'd for C₄₅H₆₀N₈O₆ 808.5; found 809.8; ¹H NMR (300 MHZ, DMSO-d₆) δ 8.66-8.45 (m, 2H), 8.29 (d, J=8.0 Hz, 1H), 7.88 (s, 1H), 7.74 (d, J=9.4 Hz, 2H), 7.30 (s, 1H), 5.81 (s, 1H), 5.00 (d, J=11.7 Hz, 1H), 4.53-4.06 (m, 5H), 3.77-3.59 (m, 2H), 3.34 (s, 4H), 3.21 (s, 3H), 3.05-2.61 (m, 5H), 2.52 (s, 4H), 2.28 (s, 3H), 2.05 (s, 1H), 1.83 (S, 1H), 1.56 (s, 2H), 1.48-1.32 (m, 3H), 1.30-0.98 (m, 12H), 0.93 (s, 3H), 0.46 (s, 3H).

### Example A396. Synthesis of (2S)—N-((6³S,3S,4S,Z)-3-ethoxy-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-((R)-1-hydroxyethyl) azetidine-1-carboxamide

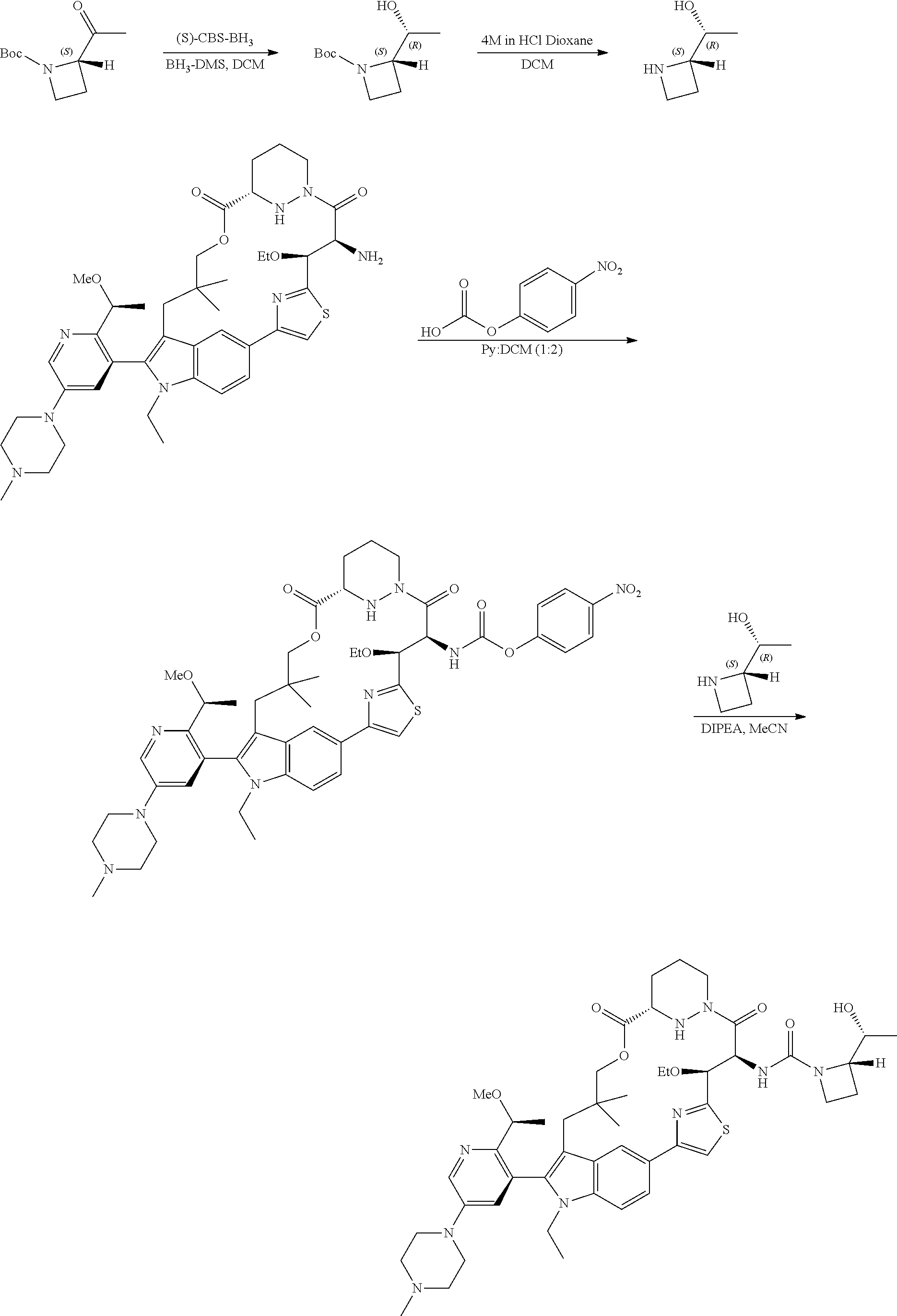

### Step 1.

To a mixture of tert-butyl(S)-2-acetylazetidine-1-carboxylate (543 mg, 1.96 mmol) in DCM (330 mL) at room temperature under an atmosphere of N₂ was added BH₃-Me₂S (2.48 g, 32.6 mmol). To the above mixture was added tert-butyl (2S)-2-acetylazetidine-1-carboxylate [Org. Lett. 2019, 22, 9981-9984-see Supporting Information] (2.6 g, 13.0 mmol) dropwise over 10 min. The resulting mixture was stirred for additional 2 h at room temperature. The reaction was monitored by LCMS. The reaction was quenched with MeOH at 0° C. and was concentrated under reduced pressure. The residue was purified by silica gel column chromatography to give tert-butyl(S)-2-((R)-1-hydroxyethyl) azetidine-1-carboxylate (1.1 g, 42% yield) as an oil. LCMS (ESI): m/z [M-C₄H₈+H]⁺ calc'd for C₆H₁₁NO₃ 145.1; found 146.1.

### Step 2.

To a mixture of tert-butyl(S)-2-((R)-1-hydroxyethyl) azetidine-1-carboxylate (300 mg, 1.49 mmol) in DCM (5 mL) at 0° C. was added 4M HCl in 1,4-dioxane (5 mL). The mixture was stirred until completion, then concentrated under reduced pressure to give (R)-1-((S)-azetidin-2-yl) ethan-1-ol (310 mg) as a solid, which was used directly in the next step without further purification. LCMS (ESI): m/z [M+H]⁺ calc'd for C₅H₁₁NO 101.1; found 102.2.

### Step 3.

To a mixture of (6³S,3S,4S,Z)-4-amino-3-ethoxy-1¹-ethyl-1²-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (150 mg, 0.19 mmol), pyridine (1 mL), and DCM (2 mL) at 0° C. was added 4-nitrophenyl chloroformate (65 mg, 0.39 mmol). The mixture was warmed to room temperature and stirred for 4 h, then concentrated under reduced pressure to give 4-nitrophenyl ((6³S,3S,4S,Z)-3-ethoxy-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (crude) as an oil, which was used directly in the next step without further purification. LCMS (ESI): m/z [M+H]⁺ calc'd for C₄₈H₅₉N₉O₉S 937.4; found 938.3.

### Step 4.

To a mixture of 4-nitrophenyl ((6³S,3S,4S,Z)-3-ethoxy-1¹-ethyl-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (195 mg, 0.21 mmol) and (R)-1-((S)-azetidin-2-yl) ethan-1-ol (285 mg, 2.8 mmol) in MeCN (2 mL) at 0° C. was added DIPEA (403 mg, 3.1 mmol) dropwise. The mixture was warmed to room temperature and stirred for 1 h. The residue was purified by preparative-HPLC to give (2S)—N-((6³S,3S,4S,Z)-3-ethoxy-1¹-ethyl-12-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-((R)-1-hydroxyethyl) azetidine-1-carboxamide (4.8 mg, 3%) as a solid. LCMS (ESI): m/z [M+H]⁺ calc'd for C₄₇H₆₅N₉O₇S 899.5; found 900.9; ¹H NMR (400 MHZ, DMSO-d₆) δ 8.52-8.45 (m, 2H), 7.91 (s, 1H), 7.76-7.72 (m, 1H), 7.58-7.53 (m, 1H), 7.22 (s, 1H), 6.73-6.68 (m, 1H), 5.54-5.50 (m, 1H), 5.31-5.26 (m, 1H), 5.16-5.12 (m, 1H), 4.91 (s, 1H), 4.30-4.24 (m, 3H), 4.16-4.04 (m, 3H), 3.84-3.67 (m, 2H), 3.69-3.41 (m, 5H), 3.28-3.23 (m, 4H), 3.16 (s, 3H), 2.84-2.79 (m, 2H), 2.48-2.44 (m, 4H), 2.23-2.21 (m, 4H), 2.08-2.05 (m, 1H), 1.93-1.89 (m, 1H), 1.79-1.77 (m, 2H), 1.54-1.52 (m, 1H), 1.36-1.33 (m, 3H), 1.26-1.16 (m, 4H), 1.13-1.10 (m, 3H), 1.00-0.73 (m, 6H), 0.43 (s, 2H).

### Examples A441 and A424. Synthesis of (1R,2R,3S)—N-((2³S,6³S,4S)-1¹-ethyl-2³-hydroxy-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)- pyridinacycloundecaphane-4-yl)-2,3-dimethylcyclopropane-1-carboxamide and (1R,2R,3S)—N-((2³R,6³S,4S)-1¹-ethyl-23-hydroxy-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)- pyridinacycloundecaphane-4-yl)-2,3-dimethylcyclopropane-1-carboxamide

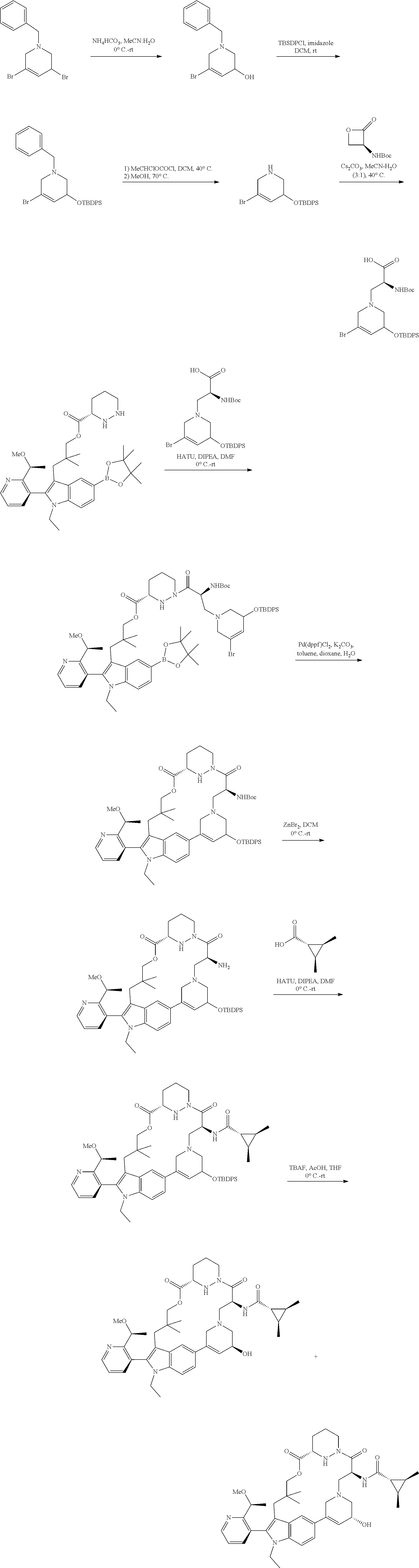

### Step 1.

To a mixture of 1-benzyl-3,5-dibromo-1,2,3,6-tetrahydropyridine (40.0 g, 120.8 mmol) in MeCN (900 mL) and H₂O (600 mL) at 0° C. was added NH₄HCO₃ (14.33 g, 181.2 mmol). The mixture was warmed to room temperature and stirred for 16 h, then concentrated under reduced pressure and the residue was purified by reverse-phase silica gel column chromatography to give 1-benzyl-5-bromo-1,2,3,6-tetrahydropyridin-3-ol (16.0 g, 49% yield) as an oil. LCMS (ESI): m/z [M+H]⁺ calc'd for C₁₂H₁₄BrNO 267.0; found 268.1.

### Step 2.

To a mixture of 1-benzyl-5-bromo-1,2,3,6-tetrahydropyridin-3-ol (15.0 g, 55.9 mmol) in DCM (150 mL) was added TBDPSCl (23.1 g, 83.9 mmol) and imidazole (7.62 g, 111.9 mmol) under an atmosphere of N₂ for 16 h. H₂O was added and the mixture and was extracted with EtOAc (3×300 mL). The combined organic layers were washed with brine (3×300 mL), dried over anhydrous Na₂SO₄ and filtered. The filtrate was concentrated under reduced pressure and the residue was purified by reverse-phase silica gel column chromatography to give 1-benzyl-5-bromo-3-((tert-butyldiphenylsilyl)oxy)-1,2,3,6-tetrahydropyridine (15.0 g, 53% yield) as an oil. LCMS (ESI): m/z [M+H]⁺ calc'd for C₂₈H₃₂BrNOSi 505.1; found 506.2.

### Step 3.

To a mixture of 1-benzyl-5-bromo-3-((tert-butyldiphenylsilyl)oxy)-1,2,3,6-tetrahydropyridine (15.0 g, 29.6 mmol) in DCM (150 mL) at 0° C. was added 2-chloroethyl chloroformate (16.93 g, 118.5 mmol). The mixture was warmed to 40° C. and was stirred for 4 h, then diluted with H₂O and the mixture was extracted with EtOAc (2×300 mL). The combined organic layers were washed with brine (2×300 mL), dried over anhydrous Na₂SO₄ and filtered. The filtrate was concentrated under reduced pressure and the residue was dissolved in MeOH (150 mL). The mixture was heated to 70° C. and stirred for 2 h, then the combined organic layers were washed with NaHCO₃ (2×300 mL), dried over anhydrous Na₂SO₄ and filtered. The filtrate was concentrated under reduced pressure and the residue was purified by reverse-phase silica gel column chromatography to give 5-bromo-3-((tert-butyldiphenylsilyl)oxy)-1,2,3,6-tetrahydropyridine (9.0 g, 73% yield) as an oil. LCMS (ESI): m/z [M+H]⁺ calc'd for C21H26BrNOSi 417.1; found 418.0 [for 81Br].

### Step 4.

To a mixture of 5-bromo-3-((tert-butyldiphenylsilyl)oxy)-1,2,3,6-tetrahydropyridine (6.0 g, 14.4 mmol) in MeCN (60 mL) and H₂O (60 mL) at 0° C. was added tert-butyl(S)-(2-oxooxetan-3-yl) carbamate (2.97 g, 15.9 mmol) and Cs₂CO₃ (11.74 g, 36.0 mmol). The mixture was warmed to 40° C. and stirred for 16 h, then extracted with EtOAc (2×100 mL). The combined organic layers were washed with brine (3×200 mL), dried over anhydrous Na₂SO₄ and filtered. The filtrate was concentrated under reduced pressure and the residue was purified by reverse-phase silica gel column chromatography to give (2S)-3-(5-bromo-3-((tert-butyldiphenylsilyl)oxy)-3,6-dihydropyridin-1 (2H)-yl)-2-((tert-butoxycarbonyl) amino) propanoic acid (4.0 g, 46% yield) as a solid. LCMS (ESI): m/z [M+H]⁺ calc'd for C₂₉H₃₉BrN₂O₅Si 602.2; found 603.1.

### Step 5.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-hexahydropyridazine-3-carboxylate (1.0 g, 1.7 mmol) in DMF (10 mL) at 0° C. was added DIPEA (1.07 g, 8.3 mmol), (2S)-3-(5-bromo-3-((tert-butyldiphenylsilyl)oxy)-3,6-dihydropyridin-1 (2H)-yl)-2-((tert-butoxycarbonyl) amino) propanoic acid (749 mg, 1.24 mmol) and HATU (1.26 g, 3.3 mmol). The mixture was stirred at 0° C. for 2 h, then diluted with H₂O, and the mixture was extracted with EtOAc (3×100 mL). The combined organic layers were washed with brine (3×100 mL), dried over anhydrous Na₂SO₄ and filtered. The filtrate was concentrated under reduced pressure and the residue was purified by preparative-TLC to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(3S)-1-((2S)-3-(5-bromo-3-((tert-butyldiphenylsilyl)oxy)-3,6-dihydropyridin-1 (2H)-yl)-2-((tert-butoxycarbonyl) amino) propanoyl) hexahydropyridazine-3-carboxylate (1.2 g, 61% yield) as an oil. LCMS (ESI): m/z [M+H]⁺ calc'd for C₆₃H₈₆BBrN₆O₉Si 1188.6; found 1189.4.

### Step 6.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(3S)-1-((2S)-3-(5-bromo-3-((tert-butyldiphenylsilyl)oxy)-3,6-dihydropyridin-1 (2H)-yl)-2-((tert-butoxycarbonyl) amino) propanoyl) hexahydropyridazine-3-carboxylate (1.2 g, 1.0 mmol) in toluene (30 mL), 1,4-dioxane (10 mL) and H₂O (10 mL) was added K₂CO₃ (418 mg, 3.0 mmol) and Pd(dppf)Cl₂ (74 mg, 0.1 mmol). The mixture was heated to 65° C. and stirred for 2 h, then diluted with H₂O, and the mixture was extracted with EtOAc (2×100 mL). The combined organic layers were washed with brine (2×100 mL), dried over anhydrous Na₂SO₄ and filtered. The filtrate was concentrated under reduced pressure and the residue was purified by preparative-TLC to give tert-butyl ((6³S,4S)-2³-((tert-butyldiphenylsilyl)oxy)-1¹-ethyl-1²-(2-((S)-1-methoxyethyl) pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)- pyridazina-2 (5,1)-pyridinacycloundecaphane-4-yl) carbamate (420 mg, 42% yield) as an oil. LCMS (ESI): m/z [M+H]⁺ calc'd for C₅₇H₇₄N₆O₇Si 982.5; found 984.1.

### Step 7.

To a mixture of tert-butyl ((6³S,4S)-2³-((tert-butyldiphenylsilyl)oxy)-1¹-ethyl-1²-(2-((S)-1-methoxyethyl) pyridin-3-yl)-10, 10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl) carbamate (420 mg, 0.43 mmol) in DCM (10 mL) at 0° C. was added ZnBr₂ (481 mg, 2.14 mmol). The mixture was warmed to room temperature and stirred for 16 h, then filtered, and the filter cake was washed with EtOAc (3×20 mL). The filtrate was concentrated under reduced pressure to give (6³S,4S)-4-amino-2³-((tert-butyldiphenylsilyl)oxy)-1¹-ethyl-1²-(2-((S)-1-methoxyethyl) pyridin-3-yl)-10,10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)- pyridinacycloundecaphane-5,7-dione (400 mg) which was used directly in the next step without further purification. LCMS (ESI): m/z [M+H]⁺ calc'd for C₅₂H₆₆N₆O₅Si 882.5; found 883.6.

### Step 8.

To a mixture of (6³S,4S)-4-amino-2³-((tert-butyldiphenylsilyl)oxy)-1¹-ethyl-1²-(2-((S)-1-methoxyethyl) pyridin-3-yl)-10, 10-dimethyl-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-5,7-dione (380 mg, 0.43 mmol) in DMF (4 mL) at 0° C. was added DIPEA (556 mg, 4.3 mmol), (1R,2R,3S)-2,3-dimethylcyclopropane-1-carboxylic acid (74 mg, 0.65 mmol) and HATU (327 mg, 0.86 mmol). The mixture was stirred at 0° C. for 2 h, then diluted with H₂O, and the mixture was extracted with EtOAc (3×50 mL). The combined organic layers were washed with brine (3×50 mL), dried over anhydrous Na₂SO₄ and filtered. The filtrate was concentrated under reduced pressure and the residue was purified by preparative-TLC to give (1R,2R,3S)—N-((6³S,4S)-2³-((tert-butyldiphenylsilyl)oxy)-1¹-ethyl-1²-(2-((S)-1-methoxyethyl) pyridin-3-yl)-10, 10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl)-2,3-dimethylcyclopropane-1-carboxamide (190 mg, 45% yield) as a solid. LCMS (ESI): m/z [M+H]⁺ calc'd for C₅₈H₇₄N₆O₆Si 978.5; found 979.7.

### Step 9.

To a mixture of (1R,2R,3S)—N-((6³S,4S)-2³-((tert-butyldiphenylsilyl)oxy)-1¹-ethyl-1²-(2-((S)-1-methoxyethyl) pyridin-3-yl)-10, 10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl)-2,3-dimethylcyclopropane-1-carboxamide (190 mg, 0.19 mmol) in THF (4 mL) at 0° C. was added TBAF (811 mg, 3.1 mmol) and AcOH (1 mg, 0.02 mmol). The mixture was warmed to room temperature and stirred for 16 h, then diluted with H₂O, and the mixture was extracted with EtOAc (3×50 mL). The combined organic layers were washed with brine (3×50 mL), dried over anhydrous Na₂SO₄ and filtered. The filtrate was concentrated under reduced pressure and the residue was purified by preparative-HPLC to give (1R,2R,3S)—N-((2³S,6³S,4S)-1¹-ethyl-2³-hydroxy-1²-(2-((S)-1-methoxyethyl) pyridin-3-yl)-10, 10-dimethyl-5,7-dioxo-2¹, 2²,2³,2⁶,6¹,6²,6³,6⁴,6⁵,6⁶-decahydro-1¹H-8-oxa-1 (5,3)-indola-6 (1,3)-pyridazina-2 (5,1)-pyridinacycloundecaphane-4-yl)-2,3-dimethylcyclopropane-1-carboxamide (26 mg, 13% yield) and (1R,2R,3S)—N-((2³R,6³S,4S)-1¹-ethyl-2³-hydroxy-1²-(2-((S)-1-methoxyethyl) pyridin-3-yl)-10, 10-dimethyl-5,7-dioxo-2¹,2²,2³,2⁶,6¹,6²,6³,6⁴,6⁵,6⁶-decahydro-1¹H-8-oxa-1 (5,3)-indola-6 (1,3)-pyridazina-2 (5,1)- pyridinacycloundecaphane-4-yl)-2,3-dimethylcyclopropane-1-carboxamide (11 mg, 8% yield) both as solids. LCMS (ESI): m/z [M+H]⁺ calc'd for C₄₂H₅₆N₆O₆ 740.4; found 741.3; ¹H NMR (400 MHZ, DMSO-d₆) δ 8.76 (dd, J=4.8, 1.7 Hz, 1H), 8.07 (d, J=8.4 Hz, 1H), 7.88 (dd, J=7.8, 1.8 Hz, 1H), 7.61-7.55 (m, 1H), 7.55-7.47 (m, 2H), 7.42 (s, 1H), 6.24 (s, 1H), 5.66 (d, J=12.2 Hz, 1H), 5.59 (t, J=9.1 Hz, 1H), 4.91 (d, J=6.7 Hz, 1H), 4.40-4.26 (m, 2H), 4.13 (q, J=6.2 Hz, 1H), 4.08-3.96 (m, 2H), 3.88-3.78 (m, 1H), 3.72 (t, J=11.5 Hz, 1H), 3.64 (q, J=11.0 Hz, 2H), 3.11 (d, J=14.2 Hz, 1H), 2.96 (dt, J=14.4, 7.1 Hz, 2H), 2.80 (d, J=23.8 Hz, 6H), 2.15 (t, J=9.5 Hz, 1H), 2.06 (d, J=8.6 Hz, 1H), 1.94 (d, J=11.0 Hz, 1H), 1.84-1.73 (m, 1H), 1.66-1.51 (m, 2H), 1.41 (d, J=6.2 Hz, 3H), 1.24 (s, 1H), 1.19-1.05 (m, 7H), 1.05-0.96 (m, 6H), 0.88 (s, 3H), 0.48 (s, 3H) and LCMS (ESI): m/z [M+H]⁺ calc'd for C₄₂H₅₆N₆O₆ 740.4; found 741.4; ¹H NMR (400 MHZ, DMSO-d₆) δ 8.76 (dd, J=4.7, 1.8 Hz, 1H), 8.12 (d, J=8.5 Hz, 1H), 7.85 (dd, J=7.8, 1.7 Hz, 1H), 7.51 (q, J=4.5 Hz, 3H), 7.45 (s, 1H), 6.19 (s, 1H), 5.70 (t, J=8.7 Hz, 1H), 5.49 (s, 1H), 4.72 (d, J=6.4 Hz, 1H), 4.31 (d, J=12.7 Hz, 1H), 4.18 (d, J=6.2 Hz, 2H), 4.14-4.06 (m, 1H), 3.99-3.68 (m, 3H), 3.61 (d, J=11.0 Hz, 1H), 3.53 (d, J=10.9 Hz, 1H), 3.14 (d, J=15.8 Hz, 1H), 2.94 (s, 3H), 2.92-2.83 (m, 2H), 2.73 (d, J=14.1 Hz, 3H), 2.33 (q, J=1.8 Hz, 1H), 1.95 (d, J=10.5 Hz, 1H), 1.77 (d, J=10.6 Hz, 1H), 1.64-1.48 (m, 2H), 1.40 (d, J=6.2 Hz, 3H), 1.24 (s, 1H), 1.19-0.95 (m, 12H), 0.85 (s, 1H), 0.72 (s, 3H), 0.58 (s, 3H).

### Example A437. Synthesis of (2S,6S)—N-((6³S,3S,4S,Z)-3-(2-(dimethylamino) ethoxy)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,4,6-trimethylpiperazine-1-carboxamide

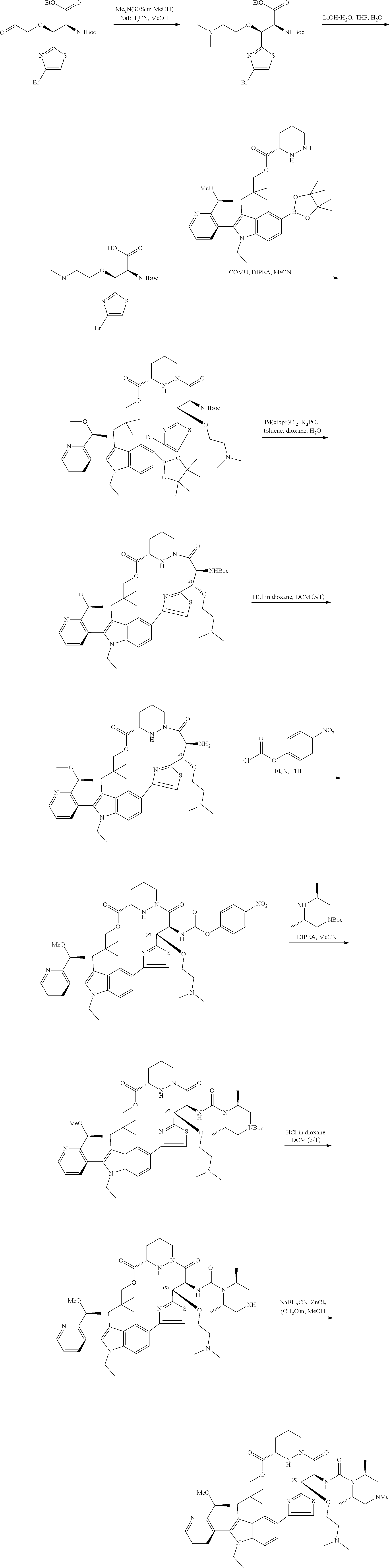

### Step 1.

To a mixture of ethyl (2S,3S)-3-(4-bromothiazol-2-yl)-2-((tert-butoxycarbonyl) amino)-3-(2-oxoethoxy) propanoate (0.31 g, 6.9 mmol) and dimethylamine, 30% in MeOH (865 mg, 8.5 mmol) in MeOH (20 mL) at 0° C. was added NaBH₃CN (1.08 g, 17.2 mmol) over 2 min. The mixture was stirred at rt, then diluted with H₂O (30 mL) and extracted with EtOAc (3×20 mL). The combined organic layers were washed with H₂O (4×10 mL), dried over anhydrous Na₂SO₄ and filtered. The filtrate was concentrated under reduced pressure and the residue was purified by silica gel column chromatography to give ethyl (2S,3S)-3-(4-bromothiazol-2-yl)-2-((tert-butoxycarbonyl) amino)-3-(2-(dimethylamino) ethoxy) propanoate (1.2 g, 45% yield) as an oil. LCMS (ESI): m/z [M+H]⁺ calc'd for C₁₇H₂₈BrN₃O₅S 467.1; found 468.2.

### Step 2.

To a mixture of ethyl (2S,3S)-3-(4-bromothiazol-2-yl)-2-((tert-butoxycarbonyl) amino)-3-(2-(dimethylamino) ethoxy) propanoate (1.25 g, 2.68 mmol) in THF (9 mL) at 0° C. under an atmosphere of N₂ was added 1M LiOH (8.0 mL, 8.0 mmol) dropwise. The mixture was stirred at 0° C. for 2 h, then acidified to PH ˜6 with 1M HCl, then extracted with EtOAc (3×50 mL). The combined organic layers were washed with brine (3×10 mL), dried over anhydrous Na₂SO₄, filtered and the filtrate was concentrated under reduced pressure to give (2S,3S)-3-(4-bromothiazol-2-yl)-2-((tert-butoxycarbonyl) amino)-3-(2-(dimethylamino) ethoxy) propanoic acid (600 mg, 51% yield) as an oil. LCMS (ESI): m/z [M+H]⁺ calc'd for C₁₅H₂₄BrN₃O₅S 439.1; found 440.3 [for 81Br].

### Step 3.

A mixture of (2S,3S)-3-(4-bromothiazol-2-yl)-2-((tert-butoxycarbonyl) amino)-3-(2-(dimethylamino) ethoxy) propanoic acid (570 mg, 1.3 mmol), 2-{[(2M)-1-ethyl-2-{2-[(1S)-1-methoxyethyl]pyridin-3-yl}-5-(4,4,5,5-tetramethyl-1,3,2-dioxaborolan-2-yl) indol-3-yl]methyl}-2-methylpropyl (3S)-1,2-diazinane-3-carboxylate (1.18 g, 1.95 mmol) and DIPEA (5.04 g, 39.0 mmol) in MECN (6 mL) at 0° C. under an atmosphere of N₂ was stirred for 5 min, then HATU (593 mg, 1.56 mmol) was added in portions over 2 min. The mixture was warmed to room temperature and stirred for 4 h, then diluted with H₂O (30 mL) and extracted with EtOAc (3×20 mL). The combined organic layers were washed with H₂O (3×10 mL), dried over anhydrous Na₂SO₄ and filtered. The filtrate was concentrated under reduced pressure and the residue was purified by preparative-TLC to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((2S,3S)-3-(4-bromothiazol-2-yl)-2-((tert-butoxycarbonyl) amino)-3-(2-(dimethylamino) ethoxy) propanoyl) hexahydropyridazine-3-carboxylate (580 mg, 44% yield) as a solid. LCMS (ESI): m/z [M+H]⁺ calc'd for C₄₉H₇₁BBrN₇O₉S 1025.4; found 1026.5 [for 81Br].

### Step 4.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((2S,3S)-3-(4-bromothiazol-2-yl)-2-((tert-butoxycarbonyl) amino)-3-(2-(dimethylamino) ethoxy) propanoyl) hexahydropyridazine-3-carboxylate (580 mg, 0.57 mmol) and K₃PO₄ (300 mg, 1.42 mmol) in toluene (6 mL), 1,4-dioxane (2 mL) and H₂O (2 mL) under an atmosphere of Ar was added Pd(dtbpf)Cl₂. The mixture was heated to 60° C. and stirred for 4 h, then diluted with H₂O (30 mL), and the mixture was extracted with EtOAc (3×30 mL). The combined organic layers were washed with brine (3×10 mL), dried over anhydrous Na₂SO₄ and filtered. The filtrate was concentrated under reduced pressure and the residue was purified by preparative-TLC to give (2S,6S)—N-((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,4,6-trimethylpiperazine-1-carboxamide (230 mg, 50% yield) as a solid. LCMS (ESI): m/z [M+H]⁺ calc'd for C₄₃H₅₉N₇O₇S 817.4; found 818.4.

### Step 5.

To a mixture of tert-butyl ((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (230 mg, 0.28 mmol) in DCM (3 mL) at 0° C. under an atmosphere of N₂ was added HCl in 1,4-dioxane (1 mL). The mixture was warmed to room temperature and stirred for 2 h, then concentrated under reduced pressure, toluene (30 mL) was added to the residue, and the mixture was concentrated under reduced pressure to give (6³S,3S,4S,Z)-4-amino-3-(2-(dimethylamino) ethoxy)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (200 mg) as a solid, which was used directly in the next step without further purification. LCMS (ESI): m/z [M+H]⁺ calc'd for C₃₈H₅₁N₇O₅S 717.4; found 718.7.

### Step 6.

To a mixture of (6³S,3S,4S,Z)-4-amino-3-(2-(dimethylamino) ethoxy)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (200 mg, 0.28 mmol) in THF (2 mL) at 0° C. was added NEt₃ (85 mg, 0.84 mmol) dropwise over 1 min. The mixture was stirred at 0° C. for 5 min, then 4-nitrophenyl chloroformate (56 mg, 0.28 mmol) was added over 1 min. The mixture was warmed to room temperature and stirred for 4 h and the mixture was concentrated under reduced pressure to give 4-nitrophenyl ((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (250 mg) as a solid, that was used directly in the next step without further purification.

### Step 7.

To a mixture of 4-nitrophenyl ((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (200 mg, 0.23 mmol) in MeCN (2 mL) at 0° C. was added DIPEA (88 mg, 0.68 mmol) dropwise. The mixture was stirred at 0° C. for 2 min, then tert-butyl (3S,5S)-3,5-dimethylpiperazine-1-carboxylate (97 mg, 0.45 mmol) was added dropwise. The mixture was warmed to room temperature and stirred overnight, then diluted with H₂O (3 mL), and the mixture was extracted with EtOAc (3×10 mL). The combined organic layers were washed with brine (3×10 mL), dried over anhydrous Na₂SO₄ and filtered. The filtrate was concentrated under reduced pressure and the residue was purified by preparative-TLC to give tert-butyl (3S,5S)-4-(((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamoyl)-3,5-dimethylpiperazine-1-carboxylate (50 mg, 23% yield) as a solid. LCMS (ESI): m/z [M+H]⁺ calc'd for C₅₀H₇₁N₉O₈S 957.5; found 958.9.

### Step 8.

To a mixture of tert-butyl (3S,5S)-4-(((6³S,3S,4S,Z)-3-(2-(dimethylamino) ethoxy)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamoyl)-3,5-dimethylpiperazine-1-carboxylate (50 mg, 0.05 mmol) in DCM (3 mL) at 0° C. under an atmosphere of N₂ was added HCl in 1,4-dioxane (1 mL) dropwise. The mixture was warmed to room temperature and stirred for 2 h, then concentrated under reduced pressure, toluene (30 mL) was added to the residue and the mixture was concentrated under reduced pressure to give (2S,6S)—N-((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,6-dimethylpiperazine-1-carboxamide (50 mg) as a solid, that was used directly in the next step without further purification.

### Step 9.

To a mixture of (2S,6S)—N-((6³S,3S,4S,Z)-3-(2-(dimethylamino) ethoxy)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,6-dimethylpiperazine-1-carboxamide (40 mg, 0.05 mmol) and MeOH at 0° C. was added paraformaldehyde (11 mg, 0.24 mmol) in portions, followed by NaBH₃CN (4.4 mg, 0.07 mmol) in portions. The mixture was warmed to room temperature and stirred overnight, then diluted with H₂O (5 mL) and extracted with EtOAc (3×10 mL). The combined organic layers were washed with H₂O (3×10 mL), dried over anhydrous Na₂SO₄ and filtered. The filtrate was concentrated under reduced pressure and the residue was purified by preparative-HPLC to give (2S,6S)—N-((6³S,3S,4S,Z)-3-(2-(dimethylamino) ethoxy)-11-ethyl-1²-(2-((S)-1-methoxyethyl) pyridin-3-yl)-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2,4,6-trimethylpiperazine-1-carboxamide (3.3 mg, 8% yield) as a solid. LCMS (ESI): m/z [M+H]⁺ calc'd for C₄₆H₆₅N₉OBS 871.5; found 872.3; ¹H NMR (400 MHZ, DMSO-d₆) δ 8.74-8.64 (dd, J=4.7, 1.6 Hz, 1H), 8.49-8.36 (s, 1H), 7.91-7.81 (s, 1H), 7.79-7.71 (d, J=7.7 Hz, 1H), 7.71-7.63 (d, J=8.6 Hz, 1H), 7.56-7.47 (s, 1H), 7.47-7.36 (dd, J=7.7, 4.7 Hz, 1H), 6.32-6.21 (d, J=9.9 Hz, 1H), 5.55-5.46 (s, 1H), 5.19-5.11 (s, 1H), 5.04-4.97 (s, 1H), 4.45-3.94 (m, 6H), 3.67-3.36 (m, 11H), 3.23 (s, 3H), 3.11 (s, 3H), 2.78-2.75 (m, 2H), 2.40-2.34 (m, 3H), 2.15 (s, 6H), 2.11-2.04 (m, 6H), 1.75 (s, 2H), 1.59-1.41 (m, 1H), 1.30 (d, J=6.0 Hz, 3H), 1.13 (s, 1H), 1.12 (d, J=6.1 Hz, 6H), 0.80 (d, J=28.4 Hz, 6H), 0.57-0.12 (s, 3H).

### Example A438. Synthesis of (1r,2R,3S)—N-((6³S,3S,4S,Z)-3-ethoxy-11-(2-isopropoxyethyl)-12-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2,3-dimethylcyclopropane-1-carboxamide

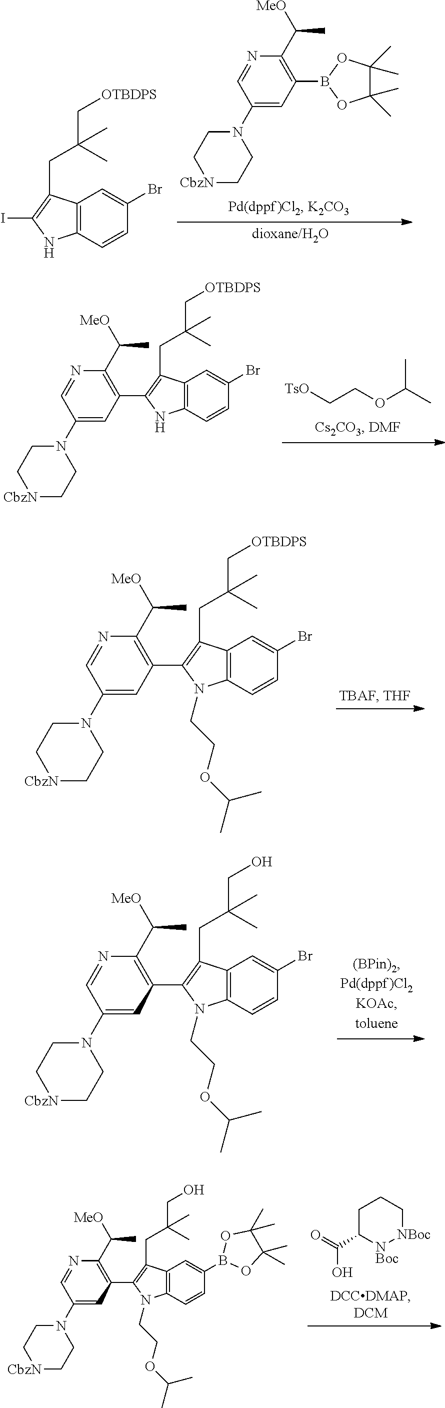

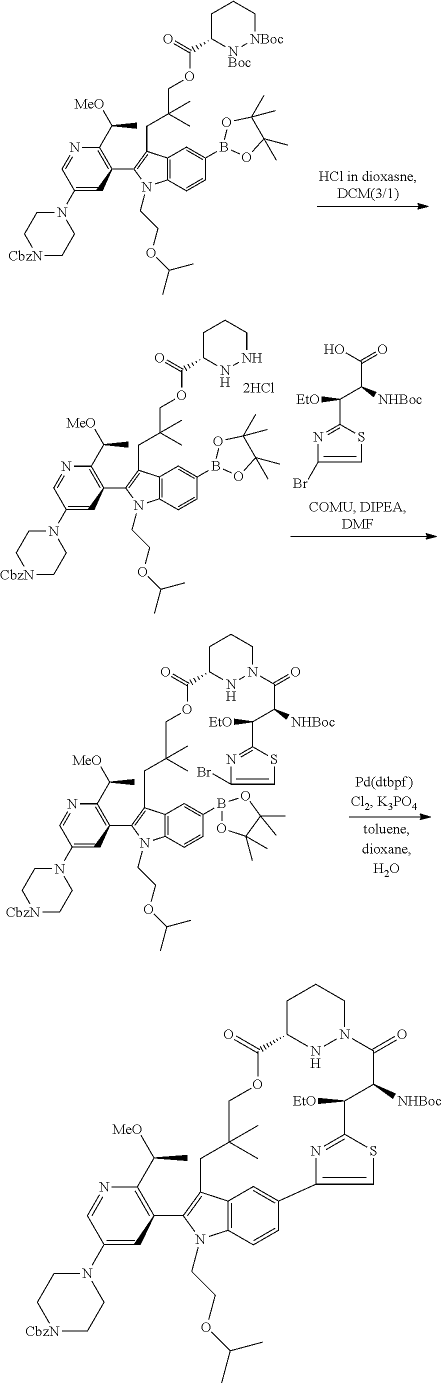

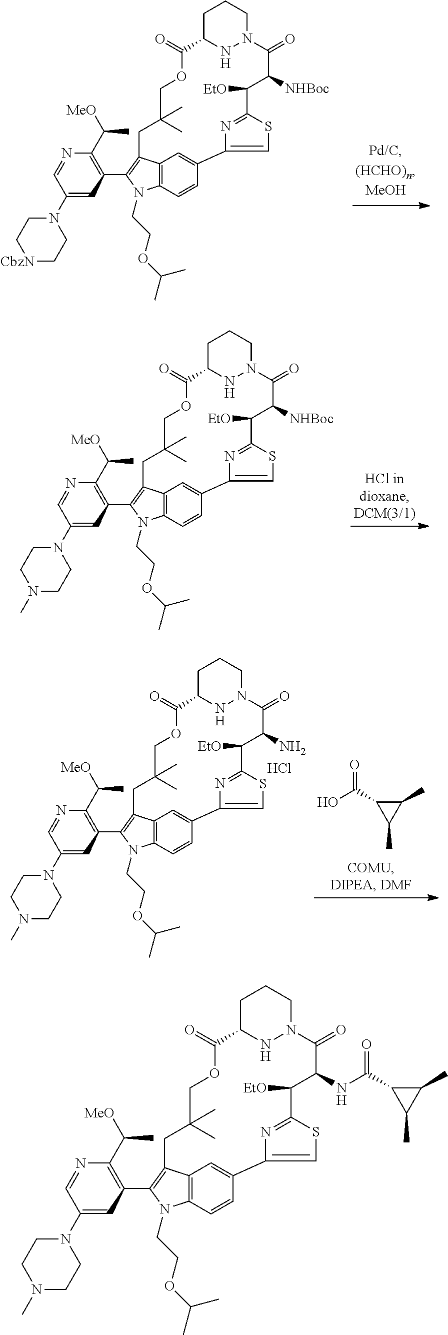

### Step 1.

To a mixture of 5-bromo-3-{3-[(tert-butyldiphenylsilyl)oxy]-2,2-dimethylpropyl}-2-iodo-1H-indole (22.0 g, 34.0 mmol) and benzyl 4-[6-(methoxymethyl)-5-(4,4,5,5-tetramethyl-1,3,2-dioxaborolan-2-yl) pyridin-3-yl]piperazine-1-carboxylate (19.1 g, 40.8 mmol) in 1,4-dioxane (400 mL) and H₂O (80 mL) under an atmosphere of N₂ was added Pd(dppf)Cl₂ (2.49 g, 3.4 mmol) and K₂CO₃ (11.76 g, 85.1 mmol) in portions. The mixture was heated to 70° C. and stirred for 16 h, then H₂O added, and the mixture was extracted with EtOAc (3×200 mL). The combined organic layers were washed with brine (3×100 mL), dried over anhydrous Na₂SO₄ and filtered. The filtrate was concentrated under reduced pressure and the residue was purified by silica gel column chromatography to give benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (20 g, 67% yield) as a solid. LCMS (ESI): m/z [M+H]⁺ calc'd for C₄₉H₅₇BrN₄O₄Si 874.3; found 875.5.

### Step 2.

To a mixture of benzyl(S)-4-(5-(5-bromo-3-(3-((tert-butyldiphenylsilyl)oxy)-2,2-dimethylpropyl)-1H-indol-2-yl)-6-(1-methoxyethyl) pyridin-3-yl) piperazine-1-carboxylate (23.0 g, 26.3 mmol) and 2-isopropoxyethyl 4-methylbenzenesulfonate (13.6 g, 52.6 mmol) in DMF (300 mL) under an atmosphere of N₂ was added Cs₂CO₃ (25.72 g, 79.0 mmol) in portions. The mixture was heated to 60° C. and stirred for 2, then diluted with brine (100 mL) and extracted with EtOAc (3×200 mL).

The combined organic layers were washed with brine (3×200 mL), dried over anhydrous Na₂SO₄ and filtered. The filtrate was concentrated under reduced pressure and the residue was purified by silica gel column chromatography to give benzyl(S)-4-(5-(5-bromo-3-(3-((tert-butyldiphenylsilyl)oxy)-2,2-dimethylpropyl)-1-(2-isopropoxyethyl)-1H-indol-2-yl)-6-(1-methoxyethyl) pyridin-3-yl) piperazine-1-carboxylate (19.2 g, 76% yield) as a solid. LCMS (ESI): m/z [M+H]⁺ calc'd for C₅₄H₆₇BrN₄O₅Si 960.4; found 961.4 [for 81Br].

### Step 3.

To a mixture of benzyl(S)-4-(5-(5-bromo-3-(3-((tert-butyldiphenylsilyl)oxy)-2,2-dimethylpropyl)-1-(2-isopropoxyethyl)-1H-indol-2-yl)-6-(1-methoxyethyl) pyridin-3-yl) piperazine-1-carboxylate (3.0 g, 3.1 mmol) in THF (30 mL) at 0° C. under an atmosphere of N₂ was added TBAF, 1M in THF (15.6 mL, 15.6 mmol) in portions. The mixture was heated to 45° C. and stirred overnight, then diluted with brine (30 mL) and extracted with EtOAc (3×100 mL). The combined organic layers were washed with brine (3×50 mL), dried over anhydrous Na₂SO₄ and filtered. The filtrate was concentrated under reduced pressure and the residue was purified by silica gel column chromatography to give benzyl(S)-4-(5-(5-bromo-3-(3-hydroxy-2,2-dimethylpropyl)-1-(2-isopropoxyethyl)-1H-indol-2-yl)-6-(1-methoxyethyl) pyridin-3-yl) piperazine-1-carboxylate (1.19 g, 53% yield) as a solid. LCMS (ESI): m/z [M+H]⁺ calc'd for C₃₈H₄₉BrN₄O₅ 720.3; found 721.3.

### Step 4.

To a mixture of benzyl(S)-4-(5-(5-bromo-3-(3-hydroxy-2,2-dimethylpropyl)-1-(2-isopropoxyethyl)-1H-indol-2-yl)-6-(1-methoxyethyl) pyridin-3-yl) piperazine-1-carboxylate (1.09 g, 1.51 mmol) and bis (pinacolato)diboron (0.58 g, 2.27 mmol) in toluene (11 mL) under an atmosphere of N₂ was added Pd(dppf)Cl₂ (0.11 g, 0.15 mmol) and KOAc (0.37 g, 3.78 mmol) in portions. The mixture was heated to 80° C. and stirred for 2 h, then diluted with brine (10 mL) and extracted with EtOAc (3×20 mL). The combined organic layers were washed with brine (3×20 mL), dried over anhydrous Na₂SO₄ and filtered. The filtrate was concentrated under reduced pressure and the residue was purified by silica gel column chromatography to give benzyl(S)-4-(5-(3-(3-hydroxy-2,2-dimethylpropyl)-1-(2-isopropoxyethyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (1.0 g, 86% yield) as a solid. LCMS (ESI): m/z [M+H]⁺ calc'd for C₄₄H₆₁BN₄O₇ 768.5; found 769.7.

### Step 5.

To a mixture of benzyl(S)-4-(5-(3-(3-hydroxy-2,2-dimethylpropyl)-1-(2-isopropoxyethyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (1.0 g, 1.3 mmol) and (3S)-1,2-bis (tert-butoxycarbonyl)-1,2-diazinane-3-carboxylic acid (0.64 g, 2.0 mmol) in DCM (10 mL) at 0° C. under an atmosphere of N₂ was added DMAP (0.24 g, 2.0 mmol) and DCC (0.40 g, 2.0 mmol) in portions. The mixture was warmed to room temperature and stirred overnight, then diluted with brine (20 mL) and extracted with DCM (3×50 mL). The combined organic layers were washed with brine (3×20 mL), dried over anhydrous Na₂SO₄ and filtered. The filtrate was concentrated under reduced pressure and the residue was purified by silica gel column chromatography to give 3-(3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl) 1,2-di-tert-butyl-(S)-tetrahydropyridazine-1,2,3-tricarboxylate (1.08 g, 77% yield) as a solid. LCMS (ESI): m/z [M+H]⁺ calc'd for C₅₉H₈₅BN₆O₁₂ 1080.6; found 1081.7.

### Step 6.

To a mixture of 3-(3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl) 1,2-di-tert-butyl-(S)-tetrahydropyridazine-1,2,3-tricarboxylate (1.0 g, 0.9 mmol) in DCM (3 mL) at 0° C. under an atmosphere of N₂ was added HCl in 1,4-dioxane (9 mL) in portions. The mixture was warmed to room temperature and stirred for 6 h, then diluted with toluene (30 mL) and concentrated under reduced pressure to give 3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl-(S)-hexahydropyridazine-3-carboxylate bis hydrochloride (1.0 g) as a solid. LCMS (ESI): m/z [M+H]⁺ calc'd for C₄₉H₆₉BN₆O₈ 880.5; found 881.5.

### Step 7.

To a mixture of 3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl-(S)-hexahydropyridazine-3-carboxylate bis hydrochloride (1.0 g, 1.1 mmol) and (2S,3S)-3-(4-bromo-1,3-thiazol-2-yl)-2-[(tert-butoxycarbonyl) amino]-3-ethoxypropanoic acid (0.54 g, 1.36 mmol) in DMF (10 mL) at 0° C. under an atmosphere of N₂ was added DIPEA (4.40 g, 34.1 mmol) and (Z)-(ethyl cyano ({[(dimethyliminiumyl) (morpholin-4-yl) methoxylimino}) formate); hexafluorophosphate (0.53 g, 1.3 mmol) in portions. The mixture was stirred at 0° C. for 1 h, then diluted with brine (30 mL) and extracted with EtOAc (3×50 mL). The combined organic layers were washed with brine (3×30 mL), dried over anhydrous Na₂SO₄ and filtered. The filtrate was concentrated under reduced pressure and the residue was purified by preparative-TLC to give 3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl-(S)-1-((2S,3S)-3-(4-bromothiazol-2-yl)-2-((tert-butoxycarbonyl) amino)-3-ethoxypropanoyl) hexahydropyridazine-3-carboxylate (1.0 g, 70% yield) as a solid. LCMS (ESI): m/z [M+H]⁺ calc'd for C₆₂H₈₆BBrN₈O₁₂S 1258.5; found 1259.5.

### Step 8.

To a mixture of 3-(2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)-2,2-dimethylpropyl-(S)-1-((2S,3S)-3-(4-bromothiazol-2-yl)-2-((tert-butoxycarbonyl) amino)-3-ethoxypropanoyl) hexahydropyridazine-3-carboxylate (1.0 g, 0.8 mmol) in toluene (30 mL), 1,4-dioxane (10 mL) and H₂O (10 mL) under an atmosphere of N₂ was added Pd(dtbpf)Cl₂ (0.16 g, 0.24 mmol) and K₃PO₄ (0.42 g, 2.0 mmol) in portions. The mixture was heated to 65° C. and stirred for 2 h, then diluted with H₂O and extracted with EtOAc (3×50 mL). The combined organic layers were washed with brine (3×50 mL), dried over anhydrous Na₂SO₄ and filtered. The filtrate was concentrated under reduced pressure and the residue was purified by preparative-TLC to give benzyl 4-(5-((6³S,3S,4S,Z)-4-((tert-butoxycarbonyl) amino)-3-ethoxy-1¹-(2-isopropoxyethyl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-12-yl)-6-((S)-1-methoxyethyl) pyridin-3-yl) piperazine-1-carboxylate (370 mg, 44% yield) as a solid. LCMS (ESI): m/z [M+H]⁺ calc'd for C₅₆H₇₄N₈O₁₀S 1050.5; found 1051.9.

### Step 9.

To a mixture of benzyl 4-(5-((6³S,3S,4S,Z)-4-((tert-butoxycarbonyl) amino)-3-ethoxy-1¹-(2-isopropoxyethyl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-1²-yl)-6-((S)-1-methoxyethyl) pyridin-3-yl) piperazine-1-carboxylate (340 mg, 0.32 mmol) in MeOH (5 mL) was added paraformaldehyde (49 mg, 1.6 mmol) and Pd/C (34 mg, 0.32 mmol) in portions. The mixture was stirred under an atmosphere of H₂ overnight, then filtered through a pad of Celite and the filter cake was washed with MeOH (3×20 mL). The filtrate was concentrated under reduced pressure and the residue was purified by preparative-TLC to give tert-butyl ((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (240 mg, 80% yield) as a solid. LCMS (ESI): m/z [M+H]⁺ calc'd for C₄₉H₇₀N₈O₈S 930.5; found 931.5.

### Step 10.

To a mixture of tert-butyl ((6₃S,3S,4S,Z)-3-ethoxy-1₁-(2-isopropoxyethyl)-1₂-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1₁H-8-oxa-2 (4,2)-thiazola-1 (5,3)-indola-6 (1,3)-pyridazinacycloundecaphane-4-yl) carbamate (240 mg, 0.26 mmol) in DCM (2 mL) at 0° C. under an atmosphere of N₂ was added HCl in 1,4-dioxane (6 mL) in portions. The mixture was stirred at 0° C. for 1 h, then diluted with toluene (20 mL) and concentrated under reduced pressure to give (6₃S,3S,4S,Z)-4-amino-3-ethoxy-1₁-(2-isopropoxyethyl)-1₂-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-1₁H-8-oxa-2 (4,2)-thiazola-1 (5,3)-indola-6 (1,3)-pyridazinacycloundecaphane-5,7-dione HCl salt (240 mg) as a solid. LCMS (ESI): m/z [M+H]⁺ calc'd for C₄₄H₆₂N₈OBS 830.5; found 831.4.

### Step 11.

To a mixture of (6³S,3S,4S,Z)-4-amino-3-ethoxy-11-(2-isopropoxyethyl)-12-(2-((S)-1-methoxyethyl)-5-(4-methylpiperazin-1-yl) pyridin-3-yl)-10, 10-dimethyl-6¹,6²,6³,6⁴,6⁵,6⁶-hexahydro-11H-8-oxa-2 (4,2)-thiazola-1 (5,3)-indola-6 (1,3)-pyridazinacycloundecaphane-5,7-dione HCl salt (100 mg, 0.12 mmol) and (1R,2R,3S)-2,3-dimethylcyclopropane-1-carboxylic acid (21 mg, 0.18 mmol) in DMF (5 mL) at 0° C. under an atmosphere of N₂ was added DIPEA (467 mg, 3.6 mmol) and (Z)-(ethyl cyano ({[(dimethyliminiumyl) (morpholin-4-yl) methoxylimino}) formate); hexafluorophophate (62 mg, 0.14 mmol) in portions. The mixture was stirred at 0° C. for 1 h, then diluted with brine (5 mL) and extracted with EtOAc (3×5 mL). The combined organic layers were washed with brine (3×15 mL), dried over anhydrous Na₂SO₄ and filtered. The filtrate was concentrated under reduced pressure and the residue was purified by (1r,2R,3S)—N-((6³S,3S,4S,Z)-3-ethoxy-11-(2-isopropoxyethyl)-12-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (38 mg, 34% yield) as a solid. LCMS (ESI): m/z [M+H]⁺ calc'd for C₅₀H₇₀N₈O₇S 926.5; found 927.5; ¹H NMR (400 MHZ, DMSO-d₆) δ 8.52-8.42 (m, 2H), 7.91 (s, 1H), 7.72 (dd, J=8.5, 1.6 Hz, 1H), 7.61 (dd, J=18.7, 9.2 Hz, 2H), 7.21 (d, J=2.9 Hz, 1H), 5.88 (d, J=9.8 Hz, 1H), 5.19 (d, J=12.2 Hz, 1H), 4.92 (s, 1H), 4.41-4.02 (m, 5H), 3.73-3.47 (m, 4H), 3.25 (q, J=4.1, 2.7 Hz, 8H), 3.13 (s, 3H), 2.78 (t, J=11.3 Hz, 2H), 2.49-2.39 (m, 6H), 2.22 (s, 4H), 2.11-1.96 (m, 1H), 1.78 (d, J=29.7 Hz, 3H), 1.51 (s, 3H), 1.32 (d, J=6.1 Hz, 4H), 1.24 (d, J=2.8 Hz, 1H), 1.20-0.97 (m, 12H), 0.84 (dd, J=23.4, 6.0 Hz, 10H), 0.44 (s, 3H).

### Example A449. Synthesis of (1r,2R,3S)—N-((6³S,3S,4S,Z)-3-ethoxy-1¹-ethyl-1²-(2-((S)-1-methoxyethyl)-5-(1-(oxetan-3-yl) piperidin-4-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

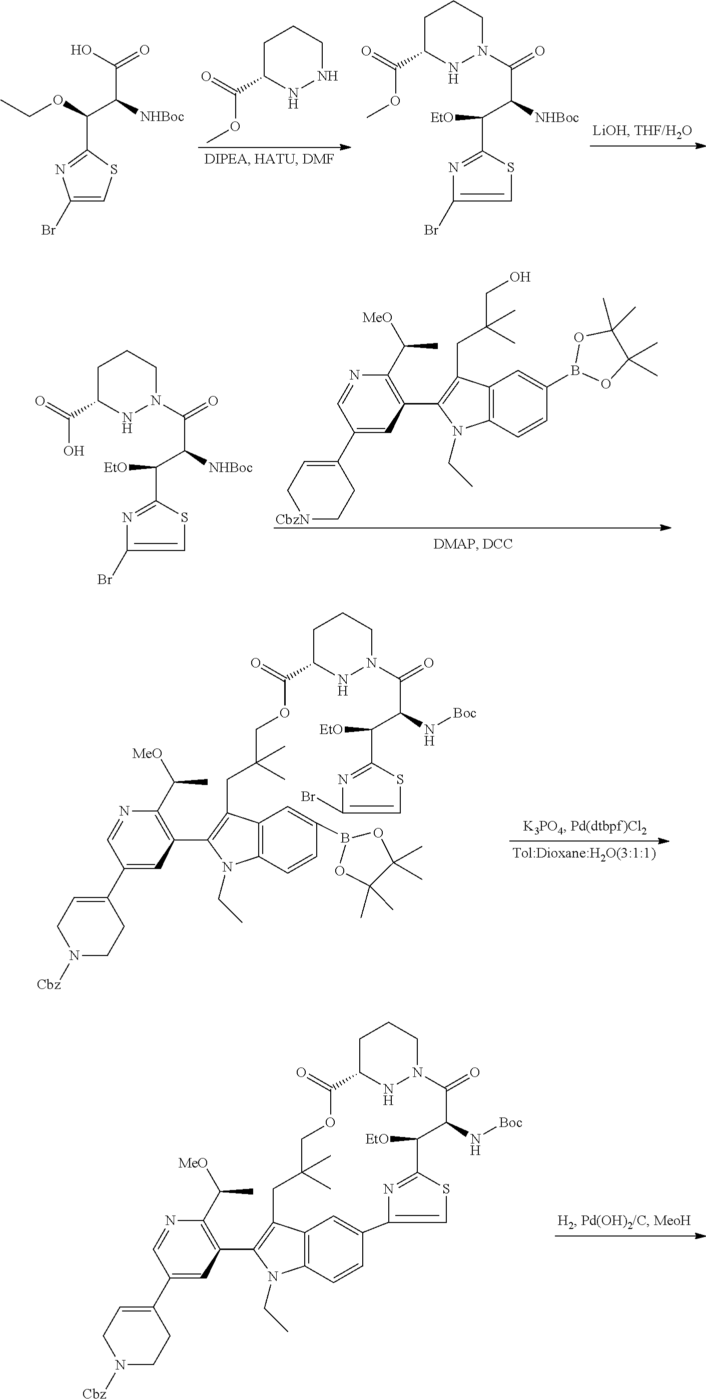

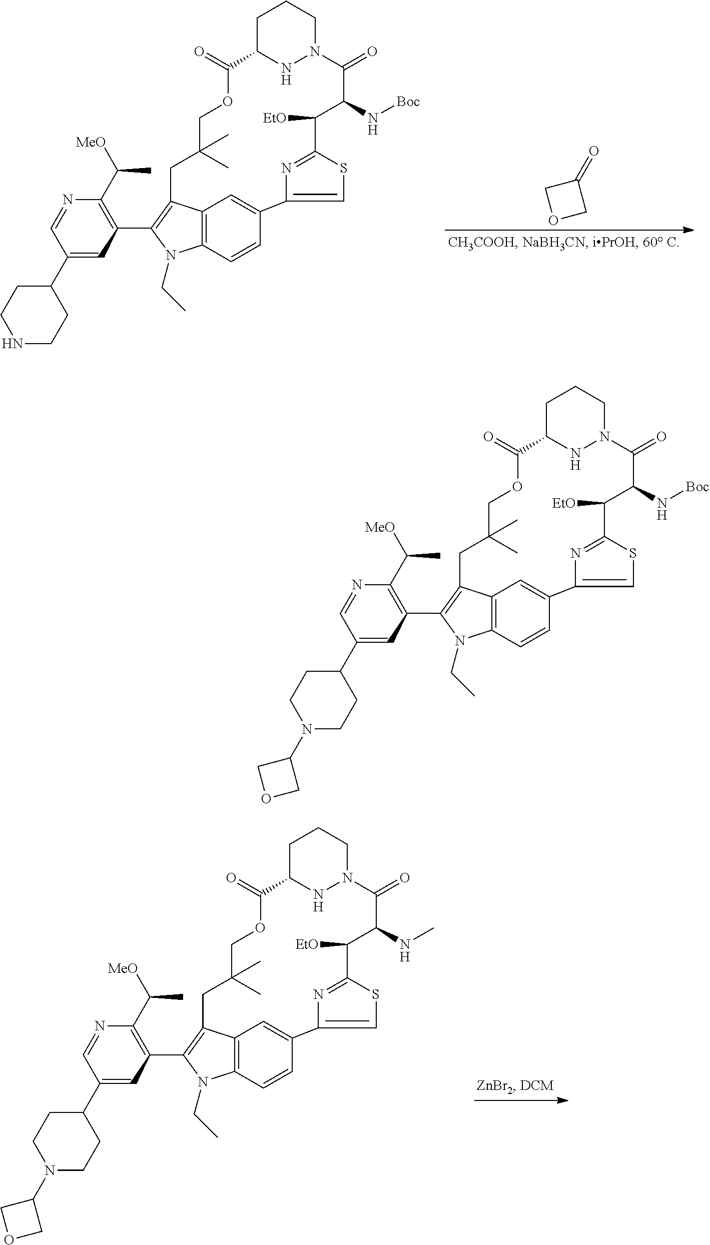

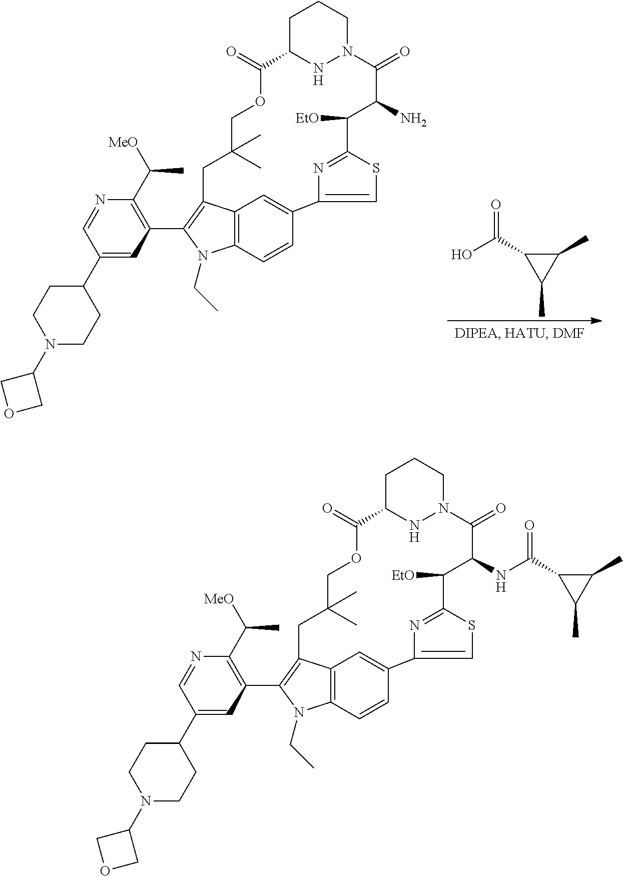

### Step 1.

A mixture of (2S,3S)-3-(4-bromothiazol-2-yl)-2-((tert-butoxycarbonyl) amino)-3-ethoxypropanoic acid (1.11 g, 7.7 mmol) and DIPEA (4.95 g, 38.3 mmol) in DMF (20 mL) was stirred at room temperature for 5 min, then (2S,3S)-3-(4-bromo-1,3-thiazol-2-yl)-2-[(tert-butoxycarbonyl) amino]-3-ethoxypropanoic acid (1.52 g, 3.8 mmol) and HATU (2.91 g, 7.7 mmol) were added. When the reaction was complete by LCMS, it was cooled to 0° C., diluted with H₂O and extracted with EtOAc (50 mL). The organic layer was washed with brine (3×100 mL), dried over anhydrous Na₂SO₄ and filtered. The filtrate was concentrated under reduced pressure and the residue was purified by silica gel column chromatography to give methyl(S)-1-((2S,3S)-3-(4-bromothiazol-2-yl)-2-((tert-butoxycarbonyl) amino)-3-ethoxypropanoyl) hexahydropyridazine-3-carboxylate (1.56 g, 78% yield). LCMS (ESI): m/z [M+H]⁺ calc'd for C₁₉H₂₉BrN₄O₆S 522.1; found 523.1 [for 81Br].

### Step 2.

A mixture of methyl (3S)-1-[(2S,3S)-3-(4-bromo-1,3-thiazol-2-yl)-2-[(tert-butoxycarbonyl) amino]-3-ethoxypropanoyl]-1,2-diazinane-3-carboxylate (1.56 g, 3.0 mmol), LIOH·H₂O (0.63 g, 15.0 mmol), THF (5 mL) and H₂O (5 mL) at room temperature was stirred until complete by LCMS. The mixture was acidified to PH ˜7 with 1N HCl was extracted with EtOAc (3×100 mL). The combined organic layers were dried over anhydrous Na₂SO₄, filtered, and the filtrate was concentrated under reduced pressure to give (3S)-1-[(2S,3S)-3-(4-bromo-1,3-thiazol-2-yl)-2-[(tert-butoxycarbonyl) amino]-3-ethoxypropanoyl]-1,2-diazinane-3-carboxylic acid (1.3 g), which was used directly in the next step without further purification. LCMS (ESI): m/z [M+H]⁺ calc'd for C₁₉H₂₉BrN₄O₆S 506.1; found 507.1.

### Step 3.

A mixture of (3S)-1-[(2S,3S)-3-(4-bromo-1,3-thiazol-2-yl)-2-[(tert-butoxycarbonyl) amino]-3-ethoxypropanoyl]-1,2-diazinane-3-carboxylic acid (600 mg, 1.18 mmol,), benzyl (5M)-5-[1-ethyl-3-(3-hydroxy-2,2-dimethylpropyl)-5-(4,4,5,5-tetramethyl-1,3,2-dioxaborolan-2-yl) indol-2-yl]-6-[(1S)-1-methoxyethyl]-3′, 6′-dihydro-2′H-[3,4′-bipyridine]-1′-carboxylate (754 mg, 1.1 mmol), DMAP (29 mg, 0.24 mmol), DCC (488 mg, 2.37 mmol) and DCM (15 mL) at room temperature until deemed complete by LCMS. The mixture was diluted with DCM (100 mL) and washed with H₂O (100 mL), then the aqueous layer was extracted with DCM (3×100 mL). The combined organic layers were dried over anhydrous Na₂SO₄, filtered, the filtrate was concentrated under reduced pressure and the residue was purified by silica gel column chromatography to give benzyl (5M)-5-(3-{3-[(3S)-1-[(2S,3S)-3-(4-bromo-1,3-thiazol-2-yl)-2-[(tert-butoxycarbonyl) amino]-3-ethoxypropanoyl]-1,2-diazinane-3-carbonyloxy]-2,2-dimethylpropyl}-1-ethyl-5-(4,4,5,5-tetramethyl-1,3,2-dioxaborolan-2-yl) indol-2-yl)-6-[(1S)-1-methoxyethyl]-3′, 6′-dihydro-2′H-[3,4′-bipyridine]-1′-carboxylate (735 mg, 52% yield) as an oil. LCMS (ESI): m/z [M+H]⁺ calc'd for C₆₀H₇₉BBrN₇O₁₁S 1195.5; found 1196.4.

### Step 4.

A mixture of benzyl (5M)-5-(3-{3-[(3S)-1-[(2S,3S)-3-(4-bromo-1,3-thiazol-2-yl)-2-[(tert-butoxycarbonyl) amino]-3-ethoxypropanoyl]-1,2-diazinane-3-carbonyloxy]-2,2-dimethylpropyl}-1-ethyl-5-(4,4,5,5-tetramethyl-1,3,2-dioxaborolan-2-yl) indol-2-yl)-6-[(1S)-1-methoxyethyl]-3′, 6′-dihydro-2′H-[3,4′-bipyridine]-1′-carboxylate (725 mg, 0.61 mmol), K₃PO₄ (322 mg, 1.52 mmol), Pd(DtBPF)Cl₂ (79 mg, 0.12 mmol), toluene (9 mL), 1,4-dioxane (3 mL) and H₂O (3 mL) under an atmosphere of N₂ was heated to 70° C. and stirred for 1 h at 70° C. The mixture was diluted with H₂O (50 mL) and extracted with EtOAc (3×100 mL). The combined organic layers were dried over anhydrous Na₂SO₄, filtered, the filtrate was concentrated under reduced pressure and the residue was purified by silica gel column chromatography to give benzyl 5-((6³S,3S,4S,Z)-4-((tert-butoxycarbonyl) amino)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl)-3′, 6′-dihydro-[3,4′-bipyridine]-1′ (2′H)-carboxylate (233 mg, 39% yield) as a an oil. LCMS (ESI): m/z [M+H]⁺ calc'd for C₅₄H₆₇N₇O₉S 989.5; found 990.6.

### Step 5.

A mixture of benzyl 5-((6³S,3S,4S,Z)-4-((tert-butoxycarbonyl) amino)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)- pyridazinacycloundecaphane-12-yl)-6-((S)-1-methoxyethyl)-3′, 6′-dihydro-[3,4′-bipyridine]-1′ (2′H)-carboxylate (223 mg, 0.23 mmol) and Pd (OH) 2/C(200 mg, 1.4 mmol) in MeOH (2 mL) was stirred under an atmosphere of H₂ until deemed complete by LCMS. The mixture was filtered, and the filter cake was washed with MeOH (3×20 mL). The filtrate was concentrated under reduced pressure to give tert-butyl ((6³S,3S,4S,Z)-3-ethoxy-1¹-ethyl-1²-(2-((S)-1-methoxyethyl)-5-(piperidin-4-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (192 mg) as a solid, that was used in the next step without further purification. LCMS (ESI): m/z [M+H]⁺ calc'd for C₄₆H₆₃N₇O₇S 875.5; found 858.4.

### Step 6.

A mixture of tert-butyl ((6³S,3S,4S,Z)-3-ethoxy-1¹-ethyl-1²-(2-((S)-1-methoxyethyl)-5-(piperidin-4-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (80 mg, 0.09 mmol), 3-oxetanone (134 mg, 1.86 mmol), AcOH (56 mg, 0.93 mmol) and NaBH₃CN (59 mg, 0.93 mmol) in PrOH (2 mL) at room temperature was stirred until deemed complete by LCMS. The mixture was cooled to 0° C. quenched with saturated NaHCO₃ and extracted with EtOAc (3×30 mL). The combined organic layers were dried over anhydrous Na₂SO₄, filtered, the filtrate was concentrated under reduced pressure and the residue was purified by silica gel column chromatography to give tert-butyl ((6³S,3S,4S,Z)-3-ethoxy-1¹-ethyl-1²-(2-((S)-1-methoxyethyl)-5-(1-(oxetan-3-yl) piperidin-4-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (30 mg, 35% yield) as a an oil. LCMS (ESI): m/z [M+H]⁺ calc'd for C₄₉H₆₇N₇O₈S 913.5; found 914.4.

### Step 7.

A mixture of tert-butyl ((6³S,3S,4S,Z)-3-ethoxy-1¹-ethyl-1²-(2-((S)-1-methoxyethyl)-5-(1-(oxetan-3-yl) piperidin-4-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (40 mg, 0.05 mmol), ZnBr₂ (55 mg, 0.24 mmol) and DCM (1 mL) was stirred at room temperature until deemed complete by LCMS. The mixture was filtered, the filter cake was washed with DCM (3×10 mL), and the filtrate was concentrated under reduced pressure to give (6³S,3S,4S,Z)-4-amino-3-ethoxy-1¹-ethyl-1²-(2-((S)-1-methoxyethyl)-5-(1-(oxetan-3-yl) piperidin-4-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dioneas (crude) an oil, which was used directly in the next step without further purification. LCMS (ESI): m/z [M+H]⁺ calc'd for C₄₄H₅₉N₇O₆S 813.4; found 814.8.

### Step 8.

To a mixture of (6³S,3S,4S,Z)-4-amino-3-ethoxy-1¹-ethyl-1²-(2-((S)-1-methoxyethyl)-5-(1-(oxetan-3-yl) piperidin-4-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dioneas (40 mg, 0.05 mmol) in DMF (2 mL) at room temperature was added DIPEA (64 mg, 0.49 mmol), (1R,2R,3S)-2,3-dimethylcyclopropane-1-carboxylic acid (6 mg, 0.05 mmol) and HATU (37 mg, 0.1 mmol) in portions. The mixture was stirred until deemed complete by LCMS. The mixture was diluted with H₂O (10 mL), extracted with EtOAc (50 mL), the organic layer was washed with brine (3×50 mL), dried over anhydrous Na₂SO₄ and filtered. The filtrate was concentrated under reduced pressure and the residue was purified by preparative-HPLC to give (1r,2R,3S)—N-((6³S,3S,4S,Z)-3-ethoxy-1¹-ethyl-1²-(2-((S)-1-methoxyethyl)-5-(1-(oxetan-3-yl) piperidin-4-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (3.5 mg, 8% yield) as a solid. LCMS (ESI): m/z [M+H]⁺ calc'd for C₅₀H₆₇N₇O₇S 909.5; found 910.8; ¹H NMR (400 MHZ, CD₃OD) δ 8.66-8.52 (m, 2H), 7.82-7.72 (m, 2H), 7.67 (s, 1H), 7.53-7.44 (m, 1H), 5.99 (s, 1H), 4.78-4.68 (m, 4H), 4.71-4.54 (m, 10H), 4.48-4.39 (m, 1H), 4.36-4.02 (m, 4H), 3.75-3.68 (m, 1H), 3.68-3.64 (m, 3H), 3.02-2.97 (m, 3H), 2.91-2.80 (m, 2H), 2.65-2.63 (m, 1H), 2.24-2.12 (m, 3H), 2.03-2.00 (m, 4H), 1.93-1.72 (m, 3H), 1.69-1.55 (m, 1H), 1.47-1.23 (m, 6H), 1.16-1.13 (m, 5H), 1.06-0.85 (m, 5H), 0.50 (s, 3H).

### Example A451. Synthesis of (1S,2S)—N-((6³S,3S,4S,Z)-1¹-ethyl-1²-(2-((S)-1-methoxyethyl) pyridin-3-yl)-10,10-dimethyl-3-((1-methylpiperidin-4-yl) methoxy)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

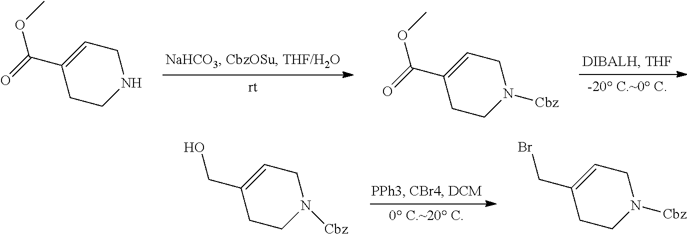

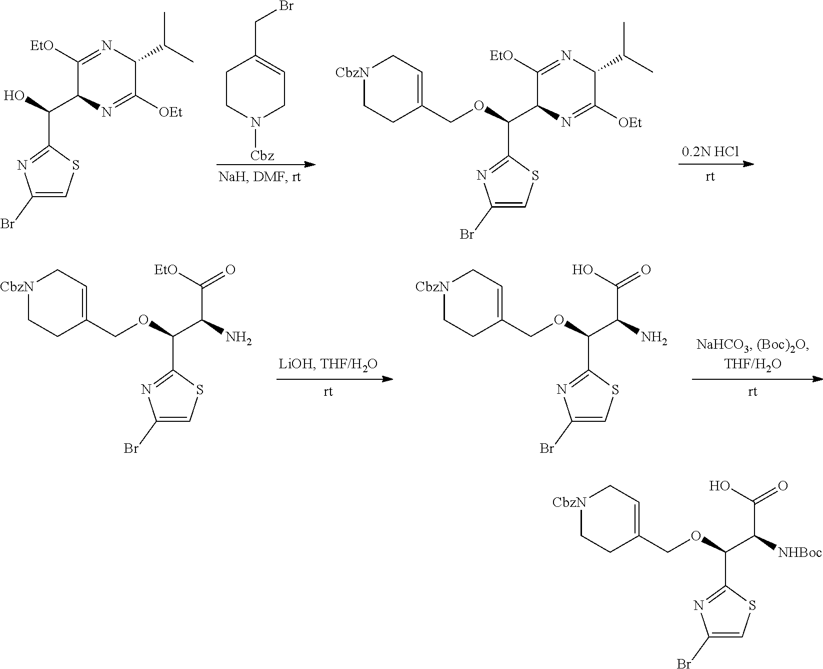

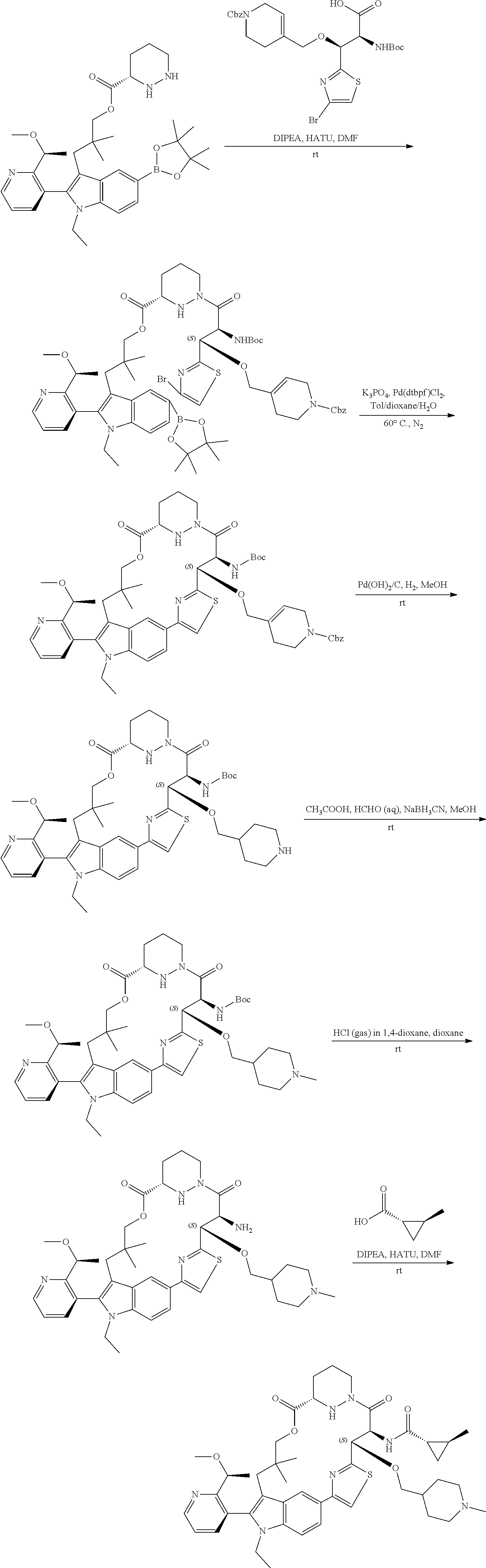

### Step 1.

To a mixture of methyl 1,2,3,6-tetrahydropyridine-4-carboxylate (10.0 g, 70.8 mmol) and NaHCO₃ (29.75 g, 354.2 mmol) in THF (50 mL) and H₂O (50 mL) was added CbzOSu (26.48 g, 106.3 mmol) in portions. The mixture was stirred at room temperature for 2 h, then washed with H₂O (3×100 mL) and the combined aqueous layers were extracted with EtOAc (3×100 mL). The combined organic layers were concentrated under reduced pressure and the residue was purified by silica gel column chromatography to give 1-benzyl 4-methyl 3,6-dihydropyridine-1,4 (2H)-dicarboxylate as an oil. LCMS (ESI): m/z [M+H]⁺ calc'd for C₁₅H₁₇NO₄ 275.1; found 276.1.

### Step 2.

To a mixture of 1-benzyl 4-methyl 3,6-dihydropyridine-1,4 (2H)-dicarboxylate (8.0 g, 29.1 mmol) in THF at −20° C. under an atmosphere of N₂ was added DIBAL-H (80 mL, 80 mmol) dropwise. The mixture was stirred at −20° C. for 2 h, then warmed to room temperature and quenched with saturated NH₄Cl. The aqueous layer was extracted with EtOAc (2×100 mL) and the combined organic layers were concentrated under reduced pressure. The residue was purified by reverse-phase silica gel column chromatography to give benzyl 4-(hydroxymethyl)-3,6-dihydropyridine-1 (2H)-carboxylate (2.3 g, 32% yield) as an oil. LCMS (ESI): m/z [M+H]⁺ calc'd for C14H17NO₃ 247.1; found 248.2.

### Step 3.

To a mixture of benzyl 4-(hydroxymethyl)-3,6-dihydropyridine-1 (2H)-carboxylate (2.3 g, 9.3 mmol) and PPh₃ (2.93 g, 11.2 mmol) in DCM at 0° C. was added CBr₄ (3.70 g, 11.2 mmol) dropwise. The mixture was stirred at room temperature until completion, then quenched with saturated NH₄Cl. The aqueous layer was extracted with EtOAc (2×30 mL), the combined organic layers were concentrated under reduced pressure and the residue was purified by silica gel column chromatography to give benzyl 4-(bromomethyl)-3,6-dihydropyridine-1 (2H)-carboxylate (2.1 g, 73% yield) as an oil. LCMS (ESI): m/z [M+H]⁺ calc'd for C₁₄H₁₆BrNO₂ 309.0; found 310.0.

### Step 4.

To a mixture of(S)-(4-bromothiazol-2-yl) ((2S,5R)-3,6-diethoxy-5-isopropyl-2,5-dihydropyrazin-2-yl) methanol (1.37 g, 3.39 mmol) in DMF (20 mL) at 0° C. was added NaH (0.16 g, 6.78 mmol) in portions. The mixture was stirred at 0° C. for 1 h, then benzyl 4-(bromomethyl)-3,6-dihydropyridine-1 (2H)-carboxylate (2.10 g, 6.78 mmol) was added. The mixture was warmed to room temperature and stirred for 2 h, then diluted with saturated NH₄Cl (100 mL), and the mixture was extracted with EtOAc (2×30 mL). The combined organic layers were concentrated under reduced pressure and the residue was purified by silica gel column chromatography to give benzyl 4-(((S)-(4-bromothiazol-2-yl) ((2,5R)-3,6-diethoxy-5-isopropyl-2,5-dihydropyrazin-2-yl) methoxy)methyl)-3,6-dihydropyridine-1 (2H)-carboxylate (1.75 g, 82% yield) as an oil. LCMS (ESI): m/z [M+H]⁺ calc'd for C₂₉H₂₇BrN₄O₅S 632.2; found 633.2.

### Step 5.

To a mixture of benzyl 4-(((S)-(4-bromothiazol-2-yl) ((2S,5R)-3,6-diethoxy-5-isopropyl-2,5-dihydropyrazin-2-yl) methoxy)methyl)-3,6-dihydropyridine-1 (2H)-carboxylate (1.75 g, 2.76 mmol) in THF (40 mL) and MECN (16 mL) at 0° C. was added 0.02M HCl (35 mL, 0.7 mmol) dropwise. The mixture was warmed to room temperature and stirred overnight, then quenched with saturated NaHCO₃ and extracted with EtOAc (2×100 mL). The combined organic layers were concentrated under reduced pressure and the residue was purified by silica gel column chromatography to give benzyl 4-(((1S,2S)-2-amino-1-(4-bromothiazol-2-yl)-3-ethoxy-3-oxopropoxy) methyl)-3,6-dihydropyridine-1 (2H)-carboxylate (1.15 g, 79% yield) as an oil. LCMS (ESI): m/z [M+H]⁺ calc'd for C₂₂H₂₆BrN₃O₅S 523.1; found 524.2.

### Step 6.

A mixture of benzyl 4-(((1 S,2S)-2-amino-1-(4-bromothiazol-2-yl)-3-ethoxy-3-oxopropoxy) methyl)-3,6-dihydropyridine-1 (2H)-carboxylate (1.15 g, 2.19 mmol) and LiOH (0.21 g, 8.77 mmol) in THF (50 mL) and H₂O (50 mL) was stirred at room temperature for 1 h, then acidified to pH ˜4 with 1M HCl. The mixture was then used directly in the next step without further purification. LCMS (ESI): m/z [M+H]⁺ calc'd for C₂₀H₂₂BrN₃O₅S 497.0; found 497.9 [for 81Br].

### Step 7.

To the above mixture was added NaHCO₃ (0.97 g, 11.59 mmol) and (Boc) 2O (1.01 g, 4.63 mmol). The mixture was stirred at room temperature overnight, then extracted with DCM (3×20 mL). The aqueous layer was acidified to PH ˜4 with 1M HCl and extracted with EtOAc (3×30 mL). The combined organic layers were concentrated under reduced pressure to give (25,3S)-3-((1-((benzyloxy) carbonyl)-1,2,3,6-tetrahydropyridin-4-yl) methoxy)-3-(4-bromothiazol-2-yl)-2-((tert-butoxycarbonyl) amino) propanoic acid (1.1 g, 79% yield) as an oil. LCMS (ESI): m/z [M+H]⁺ calc'd for C₂₅H₃₀BrN₃O₇S 597.1; found 598.0 [for 81Br].

### Step 8.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-hexahydropyridazine-3-carboxylate (1.5 g, 2.5 mmol) and (2S,3S)-3-((1-((benzyloxy) carbonyl)-1,2,3,6-tetrahydropyridin-4-yl) methoxy)-3-(4-bromothiazol-2-yl)-2-((tert-butoxycarbonyl) amino) propanoic acid (1.48 g, 2.5 mmol) in DMF was added DIPEA (3.21 g, 24.8 mmol) and HATU (1.89 g, 4.96 mmol) in portions. The mixture was stirred at room temperature for 2 h, then washed with H₂O (3×30 mL). The combined aqueous layers were extracted with EtOAc (3×30 mL), the combined organic layers were concentrated under reduced pressure and the residue was purified by silica gel column chromatography to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((2S,3S)-3-((1-((benzyloxy) carbonyl)-1,2,3,6-tetrahydropyridin-4-yl) methoxy)-3-(4-bromothiazol-2-yl)-2-((tert-butoxycarbonyl) amino) propanoyl) hexahydropyridazine-3-carboxylate (1.0 g, 34% yield) as an oil. LCMS (ESI): m/z [M+H]⁺ calc'd for C₅₉H₇₇BBrN₇O₁₁S 1183.5; found 1184.3 [for 81Br].

### Step 9.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(S)-1-((2S,3S)-3-((1-((benzyloxy) carbonyl)-1,2,3,6-tetrahydropyridin-4-yl) methoxy)-3-(4-bromothiazol-2-yl)-2-((tert-butoxycarbonyl) amino) propanoyl) hexahydropyridazine-3-carboxylate (1.0 g, 0.85 mmol) and K₃PO₄ (0.45 g, 2.11 mmol) in toluene, dioxane and H₂O under an atmosphere of N₂ was added Pd(dtbpf)Cl₂ (0.11 g, 0.17 mmol) in portions. The mixture was heated to 60° C. and stirred for 2 h, then washed with H₂O (3×30 mL) and the combined aqueous layers extracted with EtOAc (3×30 mL). The combined organic layers were concentrated under reduced pressure and the residue was purified by silica gel column chromatography to give benzyl 4-((((6³S,3S,4S,Z)-4-((tert-butoxycarbonyl) amino)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-3-yl)oxy) methyl)-3,6-dihydropyridine-1 (2H)-carboxylate (260 mg, 32% yield) as an oil. LCMS (ESI): m/z [M+H]⁺ calc'd for C₅₃H₆₅N₇O₉S 975.5; found 976.6.

### Step 10.

A mixture of benzyl 4-((((6³S,3S,4S,Z)-4-((tert-butoxycarbonyl) amino)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-3-yl)oxy) methyl)-3,6-dihydropyridine-1 (2H)-carboxylate (260 mg, 0.27 mmol) and Pd (OH) 2, 20% on carbon (0.26 g) in MeOH (3 mL) was stirred under an atmosphere of H₂ (balloon) at room temperature for 1 h. The mixture was filtered through a pad of Celite pad and the filtrate was concentrated under reduced pressure to give tert-butyl ((6³S,3S,4S,Z)-1¹-ethyl-1²-(2-((S)-1-methoxyethyl) pyridin-3-yl)-10,10-dimethyl-5,7-dioxo-3-(piperidin-4-ylmethoxy)-6¹,6²,6³,6⁴,6⁵,6⁶-hexahydro-1¹H-8-oxa-2 (4,2)-thiazola-1 (5,3)-indola-6 (1,3)- pyridazinacycloundecaphane-4-yl) carbamate (230 mg, 72% yield) as an oil. LCMS (ESI): m/z [M+H]⁺ calc'd for C₄₅H₆₁N₇O₇S 843.4; found 844.3.

### Step 11.

To a mixture of tert-butyl ((6³S,3S,4S,Z)-1¹-ethyl-1²-(2-((S)-1-methoxyethyl) pyridin-3-yl)-10, 10-dimethyl-5,7-dioxo-3-(piperidin-4-ylmethoxy)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (230 mg, 0.27 mmol) and AcOH (49 mg, 0.82 mmol) in MeOH was added paraformaldehyde (49 mg, 1.6 mmol) and NaBH₃CN (86 mg, 1.36 mmol) in portions. The mixture was stirred at room temperature for 2 h, then diluted with H₂O and extracted with EtOAc (3×20 mL). The combined organic layers were concentrated under reduced pressure and the residue was purified by silica gel column chromatography to give tert-butyl ((6³S,3S,4S, Z)-1¹-ethyl-1²-(2-((S)-1-methoxyethyl) pyridin-3-yl)-10,10-dimethyl-3-((1-methylpiperidin-4-yl) methoxy)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (280 mg, 81% yield) as a an oil. LCMS (ESI): m/z [M+H]⁺ calc'd for C₄₆H₆₃N₇O₇S 857.5; found 858.4.

### Step 12.

A mixture of tert-butyl ((6³S,3S,4S, Z)-1¹-ethyl-1²-(2-((S)-1-methoxyethyl) pyridin-3-yl)-10,10-dimethyl-3-((1-methylpiperidin-4-yl) methoxy)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (280 mg, 0.33 mmol) and HCl in 1,4-dioxane (3 mL) in 1,4-dioxane was stirred at room temperature for 1 h. The mixture was concentrated under reduced pressure to give (6³S,3S,4S,Z)-4-amino-1¹-ethyl-1²-(2-((S)-1-methoxyethyl) pyridin-3-yl)-10, 10-dimethyl-3-((1-methylpiperidin-4-yl) methoxy)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione as an oil. LCMS (ESI): m/z [M+H]⁺ calc'd for C₄₁H₅₅NO₅S 757.4; found 758.5.

### Step 13.

To a mixture of (6³S,3S,4S,Z)-4-amino-1¹-ethyl-1²-(2-((S)-1-methoxyethyl) pyridin-3-yl)-10,10-dimethyl-3-((1-methylpiperidin-4-yl) methoxy)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (265 mg, 0.35 mmol) and (1S,2S)-2-methylcyclopropane-1-carboxylic acid (35 mg, 0.35 mmol) in DMF was added DIPEA (904 mg, 7.0 mmol) in portions. The mixture was stirred at room temperature for 2 h, then washed with H₂O (3×20 mL). The combined aqueous layers were extracted with EtOAc (2×20 mL) and the combined organic layers were concentrated under reduced pressure and the residue was purified by silica gel column chromatography to give (1S,2S)—N-((6³S,3S,4S,Z)-1¹-ethyl-1²-(2-((S)-1-methoxyethyl) pyridin-3-yl)-10, 10-dimethyl-3-((1-methylpiperidin-4-yl) methoxy)-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide (24 mg, 8% yield) as a solid. LCMS (ESI): m/z [M+H]⁺ calc'd for C₄₆H₆₁N₇O₆S 839.4; found 840.4; ¹H NMR (300 MHZ, DMSO-d₆) δ 8.83-8.64 (m, 1H), 8.56-8.43 (m, 1H), 8.00-7.89 (m, 1H), 7.81-7.73 (m, 3H), 7.63-7.53 (m, 2H), 5.93-5.85 (m, 1H), 5.20-5.12 (m, 1H), 4.90 (s, 1H), 4.32-4.13 (m, 5H), 3.68-3.60 (m, 2H), 3.26-3.22 (m, 5H), 2.95-2.83 (m, 1H), 2.79-2.72 (m, 3H), 2.49-2.46 (m, 1H), 2.20-2.10 (s, 3H), 2.09-2.01 (m, 1H), 1.93-1.72 (m, 6H), 1.70-1.45 (m, 3H), 1.41-1.30 (m, 3H), 1.21-0.99 (m, 7H), 0.98-0.87 (m, 8H), 0.60-0.50 (m, 1H), 0.35 (s, 3H).

### Example A457. Synthesis of (1r,2R,3S)—N-((6³S,4S,Z)-12-(2-((S)-1-methoxyethyl) pyridin-3-yl)-10,10-dimethyl-5,7-dioxo-11-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

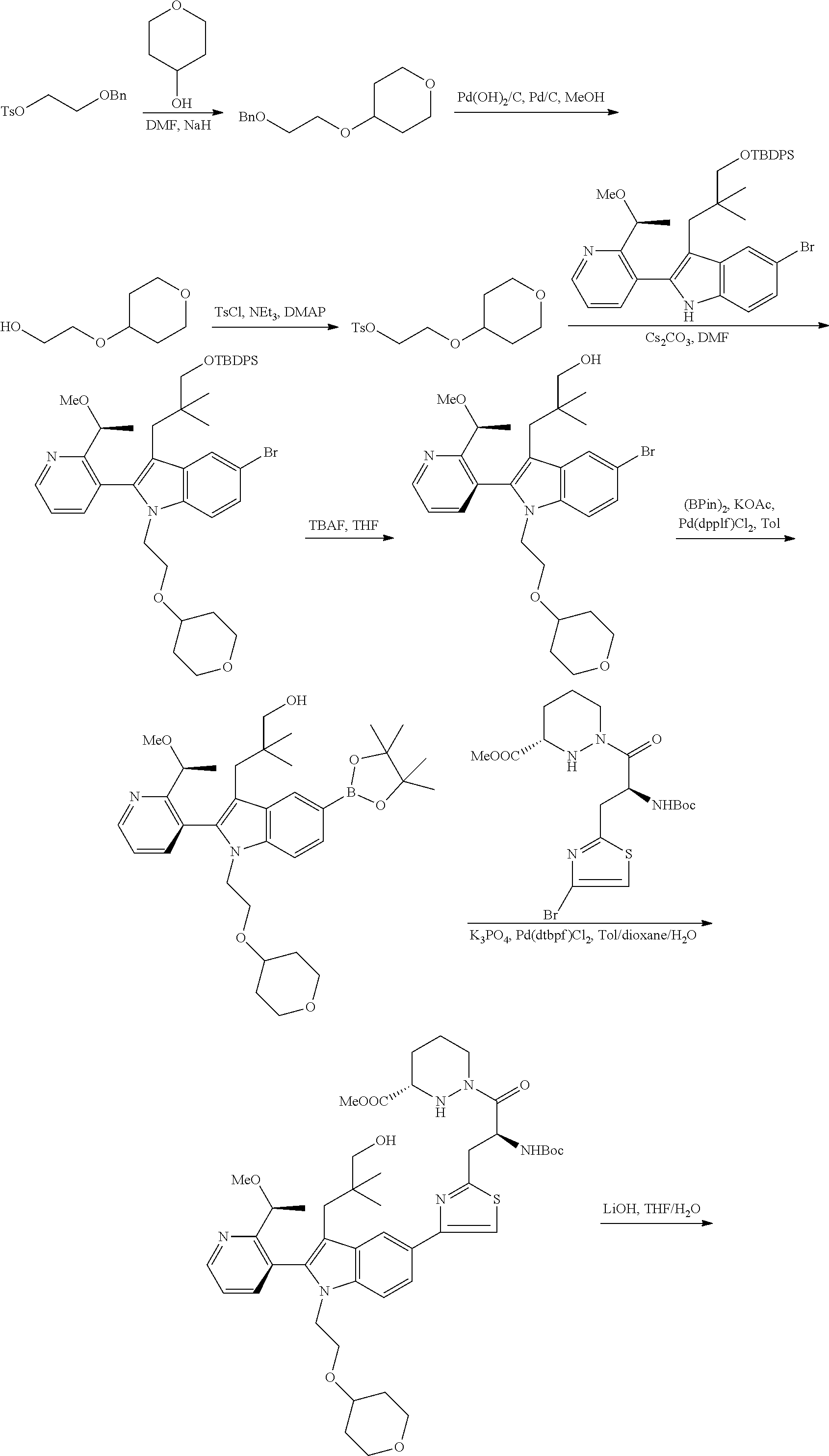

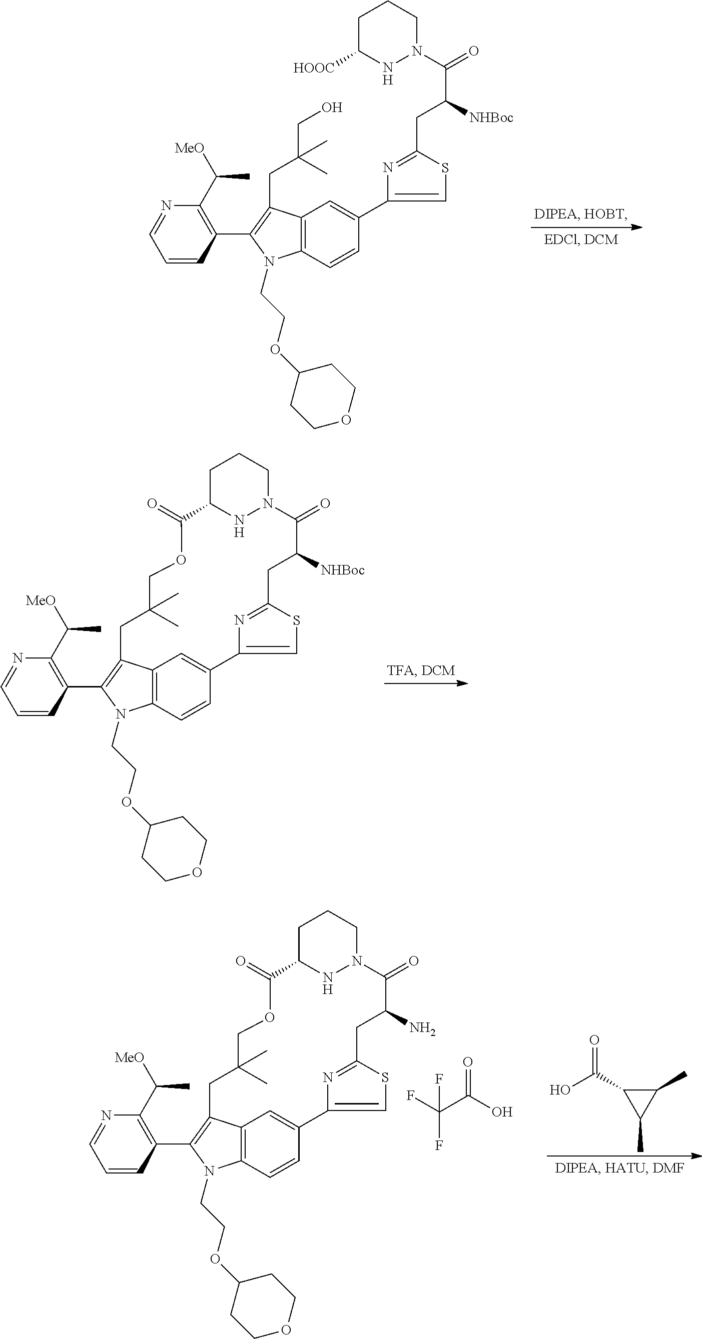

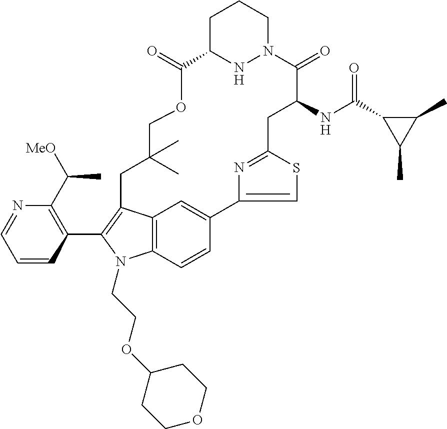

### Step 1.

To a mixture of(S)-5-bromo-3-(3-((tert-butyldiphenylsilyl)oxy)-2,2-dimethylpropyl)-2-(2-(1-methoxyethyl) pyridin-3-yl)-1H-indole (3.0 g, 4.6 mmol) and 2-((tetrahydro-2H-pyran-4-yl)oxy) ethyl 4-methylbenzenesulfonate (2.06 g, 6.9 mmol) in DMF (20 mL) at 0° C. under an atmosphere of N₂ was added Cs₂CO₃ (3.73 g, 11.4 mmol) in portions. The mixture was heated to 65° C. and stirred overnight, then diluted with H₂O (50 mL) and extracted with EtOAc (3×50 mL). The combined organic layers were washed with brine (3×50 mL), dried over anhydrous Na₂SO₄ and filtered. The filtrate was concentrated under reduced pressure and the residue was purified by silica gel column chromatography to give (S)-5-bromo-3-(3-((tert-butyldiphenylsilyl)oxy)-2,2-dimethylpropyl)-2-(2-(1-methoxyethyl) pyridin-3-yl)-1-(2-((tetrahydro-2H-pyran-4-yl)oxy) ethyl)-1H-indole (2.3 g, 64% yield) as a solid. LCMS (ESI): m/z [M+H]⁺ calc'd for C₄₄H₅₅BrN₂O₄Si 784.3; found 785.2.

### Step 2.

To a mixture of(S)-5-bromo-3-(3-((tert-butyldiphenylsilyl)oxy)-2,2-dimethylpropyl)-2-(2-(1-methoxyethyl) pyridin-3-yl)-1-(2-((tetrahydro-2H-pyran-4-yl)oxy) ethyl)-1H-indole (2.3 g, 2.9 mmol) in THF (20 mL) at 0° C. under an atmosphere of N₂ was added TBAF, 1M in THF (14.67 mL, 14.7 mmol) in portions. The mixture was heated to 45° C. and stirred for 6 h, then diluted with H₂O and extracted with EtOAc (3×30 mL). The combined organic layers were washed with brine (3×30 mL), dried over anhydrous Na₂SO₄ and filtered. The filtrate was concentrated under reduced pressure and the residue was purified by preparative-TLC to give (S)-3-(5-bromo-2-(2-(1-methoxyethyl) pyridin-3-yl)-1-(2-((tetrahydro-2H-pyran-4-yl)oxy) ethyl)-1H-indol-3-yl)-2,2-dimethylpropan-1-ol (690 mg, 43% yield) as a solid. LCMS (ESI): m/z [M+H]⁺ calc'd for C₂₈H₃₇BrN₂O₄ 546.2; found 546.9.

### Step 3.

To a mixture of(S)-3-(5-bromo-2-(2-(1-methoxyethyl) pyridin-3-yl)-1-(2-((tetrahydro-2H-pyran-4-yl)oxy) ethyl)-1H-indol-3-yl)-2,2-dimethylpropan-1-ol (690 mg, 1.3 mmol) and (Bpin) 2 (643 mg, 2.5 mmol) in toluene (7 mL) under an atmosphere of Ar was added KOAc (372 mg, 3.8 mmol) and Pd(dppf)Cl₂ (93 mg, 0.13 mmol) in portions. The mixture was heated to 80° C. and stirred for 2.5 h, then diluted with H₂O (20 mL) and extracted with EtOAc (3×20 mL). The combined organic layers were washed with brine (3×30 mL), dried over anhydrous Na₂SO₄ and filtered. The filtrate was concentrated under reduced pressure and the residue was purified by preparative-TLC to give (S)-3-(2-(2-(1-methoxyethyl) pyridin-3-yl)-1-(2-((tetrahydro-2H-pyran-4-yl)oxy) ethyl)-5-(4,4,5,5-tetramethyl-1,3,2-dioxaborolan-2-yl)-1H-indol-3-yl)-2,2-dimethylpropan-1-ol (700 mg, 93% yield) as a solid. LCMS (ESI): m/z [M+H]⁺ calc'd for C₃₄H₄₉BN₂O₆ 592.4; found 593.1.

### Step 4.

To a mixture of(S)-3-(2-(2-(1-methoxyethyl) pyridin-3-yl)-1-(2-((tetrahydro-2H-pyran-4-yl)oxy) ethyl)-5-(4,4,5,5-tetramethyl-1,3,2-dioxaborolan-2-yl)-1H-indol-3-yl)-2,2-dimethylpropan-1-ol (670 mg, 1.13 mmol) and Pd(DtBPF)Cl₂ (147 mg, 0.23 mmol) in toluene (3 mL), 1,4-dioxane (1 mL) and H₂O (1 mL) under an atmosphere of Ar was added methyl(S)-1-((S)-3-(4-bromothiazol-2-yl)-2-((tert-butoxycarbonyl) amino) propanoyl) hexahydropyridazine-3-carboxylate (648 mg, 1.36 mmol) and K₃PO₄ (720 mg, 3.39 mmol) in portions. The mixture was heated to 60° C. and stirred for 3 h, then diluted with H₂O (10 mL) and extracted with EtOAc (3×10 mL). The combined organic layers were washed with brine (3×10 mL), dried over anhydrous Na₂SO₄ and filtered. The filtrate was concentrated under reduced pressure and the residue was purified by preparative-TLC to give methyl(S) -1-((S)-2-((tert-butoxycarbonyl) amino)-3-(4-(3-(3-hydroxy-2,2-dimethylpropyl)-2-(2-((S)-1-methoxyethyl) pyridin-3-yl)-1-(2-((tetrahydro-2H-pyran-4-yl)oxy) ethyl)-1H-indol-5-yl) thiazol-2-yl) propanoyl) hexahydropyridazine-3-carboxylate (680 mg, 70% yield) as a solid. LCMS (ESI): m/z [M+H]⁺ calc'd for C₄₅H₆₂N₆O₉S 862.4; found 863.1.

### Step 5.

To a mixture of methyl(S)-1-((S)-2-((tert-butoxycarbonyl) amino)-3-(4-(3-(3-hydroxy-2,2-dimethylpropyl)-2-(2-((S)-1-methoxyethyl) pyridin-3-yl)-1-(2-((tetrahydro-2H-pyran-4-yl)oxy) ethyl)-1H-indol-5-yl) thiazol-2-yl) propanoyl) hexahydropyridazine-3-carboxylate (300 mg, 0.35 mmol) in THF (6 mL) at 0° C. under an atmosphere of N₂ was added 1M LiOH (1.74 mL, 1.74 mmol) in portions. When the reaction was deemed complete by LCMS the mixture was acidified to PH ˜6 with 1M HCl, then extracted with EtOAc (3×50 mL). The combined organic layers were washed with brine (2×20 mL), dried over anhydrous Na₂SO₄, filtered and the filtrate was concentrated under reduced pressure to give (S)-1-((S)-2-((tert-butoxycarbonyl) amino)-3-(4-(3-(3-hydroxy-2,2-dimethylpropyl)-2-(2-((S)-1-methoxyethyl) pyridin-3-yl)-1-(2-((tetrahydro-2H-pyran-4-yl)oxy) ethyl)-1H-indol-5-yl) thiazol-2-yl) propanoyl) hexahydropyridazine-3-carboxylic acid (280 mg) as a solid, which was used directly in the next step without further purification. LCMS (ESI): m/z [M+H]⁺ calc'd for C₄₄H₆₀N₆O₉S 848.4; found 849.4.

### Step 6.

To a mixture of(S)-1-((S)-2-((tert-butoxycarbonyl) amino)-3-(4-(3-(3-hydroxy-2,2-dimethylpropyl)-2-(2-((S)-1-methoxyethyl) pyridin-3-yl)-1-(2-((tetrahydro-2H-pyran-4-yl)oxy) ethyl)-1H-indol-5-yl) thiazol-2-yl) propanoyl) hexahydropyridazine-3-carboxylic acid (300 mg, 0.35 mmol) and DIPEA (457 mg, 3.5 mmol) in DCM (50 mL) at 0° C. under an atmosphere of N₂ was added HOBT (477 mg, 3.5 mmol) and EDCI (2.03 g, 10.6 mmol) in portions. The mixture was warmed to room temperature and stirred overnight, then washed with H₂O (50 mL) and the aqueous layer was extracted with DCM (3×50 mL). The combined organic layers were washed with brine (3×100 mL), dried over anhydrous Na₂SO₄ and filtered. The filtrate was concentrated under reduced pressure and the residue was purified by preparative-TLC to give tert-butyl ((6³S,4S,Z)-12-(2-((S)-1-methoxyethyl) pyridin-3-yl)-10, 10-dimethyl-5,7-dioxo-11-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (200 mg, 68% yield) as a solid. LCMS (ESI): m/z [M+H]⁺ calc'd for C₄₄H₅₈N₆O₈S 830.4; found 831.3.

### Step 7.

To a mixture of tert-butyl ((6³S,4S,Z)-12-(2-((S)-1-methoxyethyl) pyridin-3-yl)-10, 10-dimethyl-5,7-dioxo-11-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (90 mg, 0.11 mmol) in DCM (1 mL) at 0° C. under an atmosphere of N₂ was added TFA (1 mL) in portions. The mixture was stirred at 0° C. for 1.5 h, then concentrated under reduced pressure to give (6³S,4S,Z)-4-amino-12-(2-((S)-1-methoxyethyl) pyridin-3-yl)-10, 10-dimethyl-11-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione TFA salt (80 mg) as an oil, that was used directly in the next step without further purification. LCMS (ESI): m/z [M+H]⁺ calc'd for C₃₉H₅₀N₆O₆S 730.4; found 731.4.

### Step 8.

To a mixture of (6³S,4S,Z)-4-amino-12-(2-((S)-1-methoxyethyl) pyridin-3-yl)-10, 10-dimethyl-11-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione TFA salt (90 mg, 0.12 mmol) and (1r,2R,3S)-2,3-dimethylcyclopropane-1-carboxylic acid (17 mg, 0.15 mmol) at 0° C. under an atmosphere of N₂ was added DIPEA (318 mg, 2.5 mmol) and HATU (56 mg, 0.15 mmol) in portions. The mixture was stirred at 0° C. for 1 h, then washed with H₂O (1 mL) and extracted with EtOAc (3×30 mL). The combined organic layers were washed with brine (30 mL), dried over anhydrous Na₂SO₄ and filtered. The filtrate was concentrated under reduced pressure and the residue was purified by preparative-HPLC to give (1r,2R,3S)—N-((6³S,4S,Z)-1²-(2-((S)-1-methoxyethyl) pyridin-3-yl)-10,10-dimethyl-5,7-dioxo-1¹-(2-((tetrahydro-2H-pyran-4-yl)oxy) ethyl)-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (17 mg, 17% yield) as a solid. LCMS (ESI): m/z [M+H]⁺ calc'd for C₄₅H₅₈N₆O₇S 826.4; found 827.6; ¹H NMR (400 MHZ, DMSO-d₆) δ 8.74 (dd, J=4.7, 1.8 Hz, 1H), 8.48 (d, J=1.6 Hz, 1H), 8.39 (d, J=9.0 Hz, 1H), 7.81 (s, 1H), 7.74 (ddd, J=10.5, 8.2, 1.8 Hz, 2H), 7.61 (d, J=8.7 Hz, 1H), 7.51 (dd, J=7.7, 4.7 Hz, 1H), 5.56 (t, J=9.0 Hz, 1H), 5.07 (d, J=12.1 Hz, 1H), 4.46 (dt, J=14.9, 5.3 Hz, 1H), 4.32-4.09 (m, 4H), 3.62-3.47 (m, 4H), 3.34-3.30 (m, 1H), 3.30-3.28 (m, 1H), 3.26 (s, 3H), 3.21-3.04 (m, 4H), 2.94 (d, J=14.3 Hz, 1H), 2.83-2.68 (m, 1H), 2.46-2.43 (m, 1H), 2.16-2.05 (m, 1H), 1.76 (d, J=20.9 Hz, 2H), 1.51 (d, J=13.5 Hz, 3H), 1.36 (d, J=6.1 Hz, 3H), 1.30-1.09 (m, 3H), 1.13-0.98 (m, 8H), 0.88 (s, 3H), 0.32 (s, 3H).

### Example A459. Synthesis of (2S)—N-((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-(methoxymethyl) azetidine-1-carboxamide

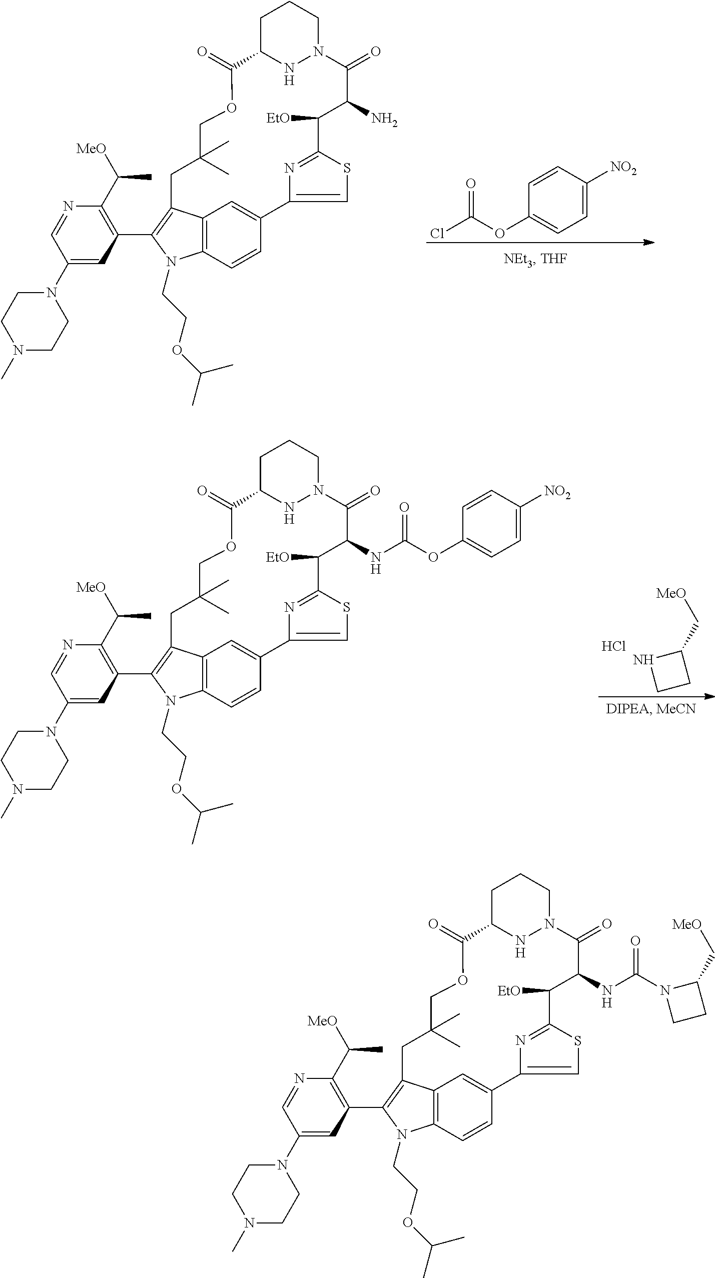

### Step 1.

A mixture of (6³S,3S,4S,Z)-4-amino-3-ethoxy-1¹-(2-isopropoxyethyl)-1²-(2-((S)-1-methoxyethyl)-5-(4-methylpiperazin-1-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (170 mg, 0.21 mmol) in THF (6 mL) at 0° C. under an atmosphere of N₂ was added TEA (62 mg, 0.62 mmol) and 4-nitrophenyl chloroformate (62 mg, 0.31 mmol) in portions. The mixture was warmed to room temperature and stirred for 4 h, then concentrated under reduced pressure to give 4-nitrophenyl ((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (200 mg) as a solid, which was used directly in the next step without further purification.

### Step 2.

To a mixture of 4-nitrophenyl ((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (150 mg, 0.15 mmol) in MeCN (8 mL) at 0° C. under an atmosphere of N₂ was added DIPEA (78 mg, 0.60 mmol) and (2S)-2-(methoxymethyl) azetidine HCl salt (46 mg, 0.45 mmol) in portions. The mixture was warmed to room temperature and stirred overnight, then the mixture was diluted with brine (10 mL) and extracted with EtOAc (3×20 mL). The combined organic layers were washed with brine (3×20 mL), dried over anhydrous Na₂SO₄ and filtered. The filtrate was concentrated under reduced pressure and the residue was purified by preparative-HPLC to give (2S)—N-((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-(methoxymethyl) azetidine-1-carboxamide (56 mg, 39% yield) as a solid. LCMS (ESI): m/z [M+H]⁺ calc'd for C₅₀H₇₁N₉O₈S 957.5; found 958.4; 1H NMR (400 MHZ, DMSO-d₆) δ 8.47 (dd, J=14.9, 2.2 Hz, 2H), 7.91 (s, 1H), 7.74 (dd, J=8.6, 1.6 Hz, 1H), 7.59 (d, J=8.7 Hz, 1H), 7.21 (s, 1H), 6.83 (d, J=10.2 Hz, 1H), 5.51 (d, J=10.2 Hz, 1H), 5.16 (d, J=12.0 Hz, 1H), 4.90 (s, 1H), 4.37 (s, 2H), 4.27-4.00 (m, 4H), 3.71 (q, J=8.4 Hz, 1H), 3.65-3.54 (m, 5H), 3.50 (q, J=9.4, 8.4 Hz, 2H), 3.43 (s, 3H), 3.40 (s, 2H), 3.30 (s, 1H), 3.29-3.20 (m, 4H), 3.13 (s, 3H), 2.90-2.72 (m, 2H), 2.67 (s, 1H), 2.43 (s, 2H), 2.23 (s, 4H), 2.05 (d, J=12.0 Hz, 1H), 1.82 (dd, J=23.8, 13.9 Hz, 3H), 1.52 (d, J=12.1 Hz, 1H), 1.33 (d, J=6.1 Hz, 3H), 1.13 (t, J=7.0 Hz, 3H), 0.93-0.84 (m, 6H), 0.81 (d, J=6.1 Hz, 3H), 0.44 (s, 3H).

### Example A460. Synthesis of (2R)—N-((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylazetidine-1-carboxamide

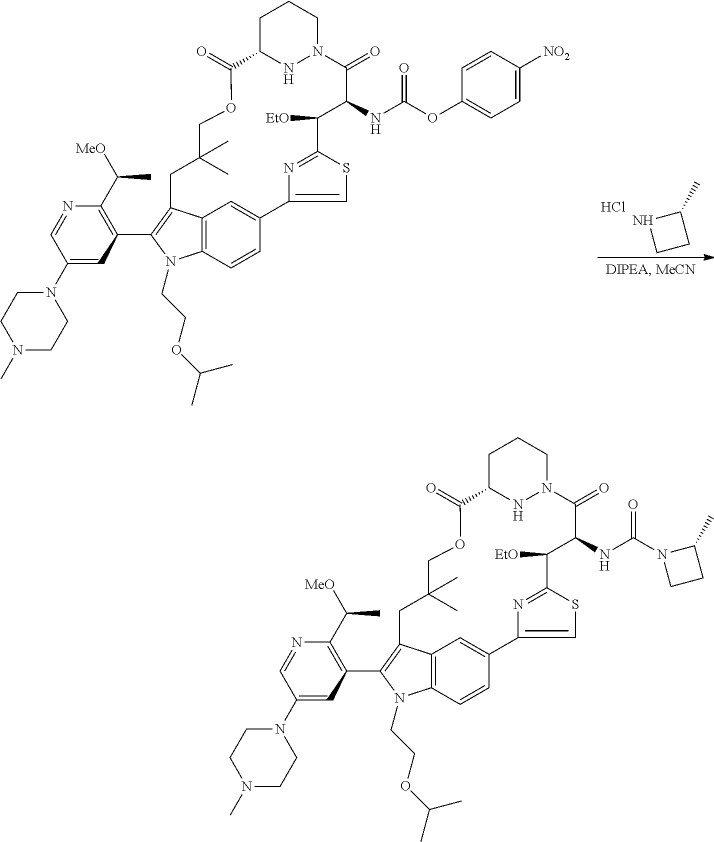

### Step 1.

To a mixture of 4-nitrophenyl ((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (150 mg, 0.15 mmol) in MECN (8 mL) at 0° C. under an atmosphere of N₂ was added DIPEA (78 mg, 0.60 mmol) and (2R)-2-methylazetidine (32 mg, 0.45 mmol) in portions. The mixture was warmed to room temperature and stirred overnight, then diluted with brine (10 mL) and extracted with EtOAc (3×20 mL). The combined organic layers were washed with brine (3×20 mL), dried over anhydrous Na₂SO₄ and filtered. The filtrate was concentrated under reduced pressure and the residue was purified by preparative-HPLC to give (2R)—N-((6³S,3S,4S,Z)-3-ethoxy-1¹-(2-isopropoxyethyl)-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylazetidine-1-carboxamide (57 mg, 41% yield) as a solid. LCMS (ESI): m/z [M+H]⁺ calc'd for C₄₉H₆₉N₉O₇S 927.5; found 928.4; ¹H NMR (400 MHZ, DMSO-d₆) δ 8.53-8.40 (m, 2H), 7.93 (s, 1H), 7.78-7.69 (m, 1H), 7.59 (d, J=8.6 Hz, 1H), 7.21 (s, 1H), 5.56 (d, J=10.2 Hz, 1H), 5.21 (d, J=12.1 Hz, 1H), 5.12 (d, J=10.2 Hz, 1H), 4.91 (s, 1H), 4.35 (d, J=13.9 Hz, 1H), 4.27-4.12 (m, 4H), 4.08 (d, J=6.1 Hz, 1H), 3.75 (t, J=7.4 Hz, 2H), 3.65-3.46 (m, 4H), 3.30 (s, 2H), 3.29 (s, 1H), 3.26 (d, J=5.9 Hz, 5H), 3.13 (s, 3H), 2.79 (d, J=13.5 Hz, 2H), 2.60 (s, 2H), 2.45 (p, J=1.9 Hz, 4H), 2.22 (s, 3H), 2.06 (d, J=12.1 Hz, 1H), 1.82 (s, 3H), 1.54-1.49 (m, 1H), 1.34 (dd, J=14.9, 6.2 Hz, 6H), 1.13 (t, J=7.0 Hz, 3H), 0.87 (t, J=6.8 Hz, 6H), 0.81 (d, J=6.0 Hz, 3H), 0.44 (s, 3H).

### Example A476. Synthesis of (1R,2R,3S)—N-((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl)-2,3-dimethylcyclopropane-1-carboxamide and (1R,2R,3S)—N-((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl)-2,3-dimethylcyclopropane-1-carboxamide

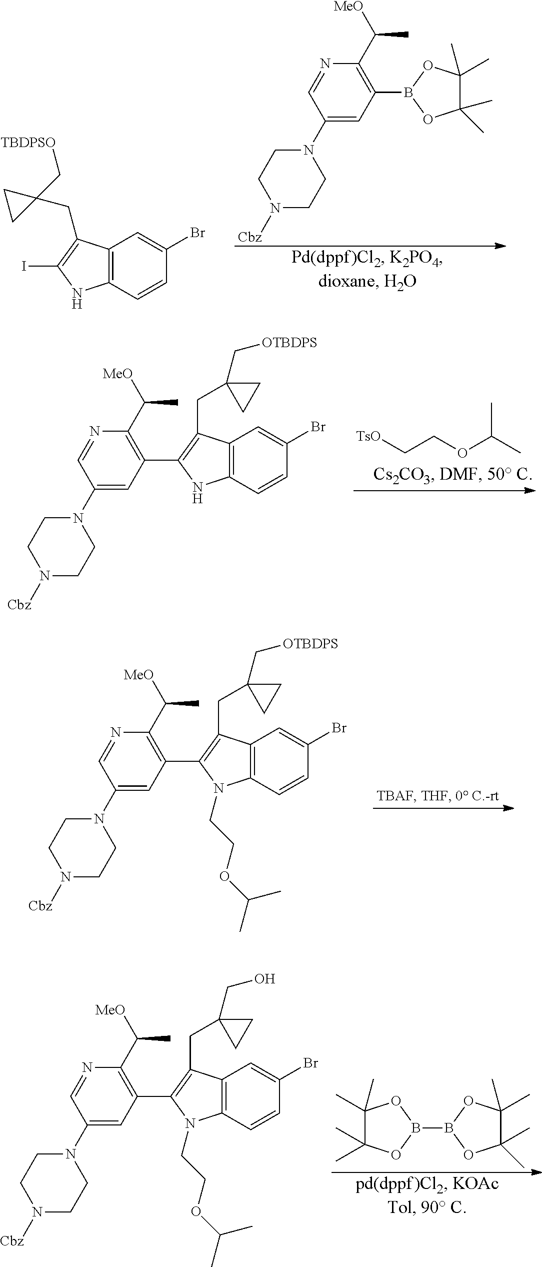

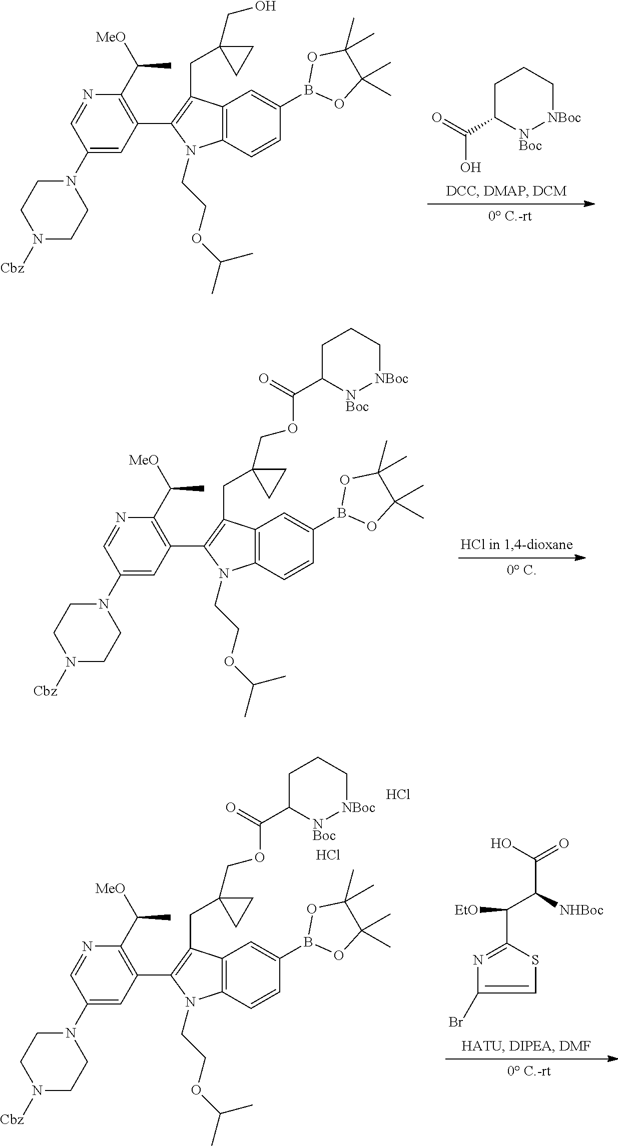

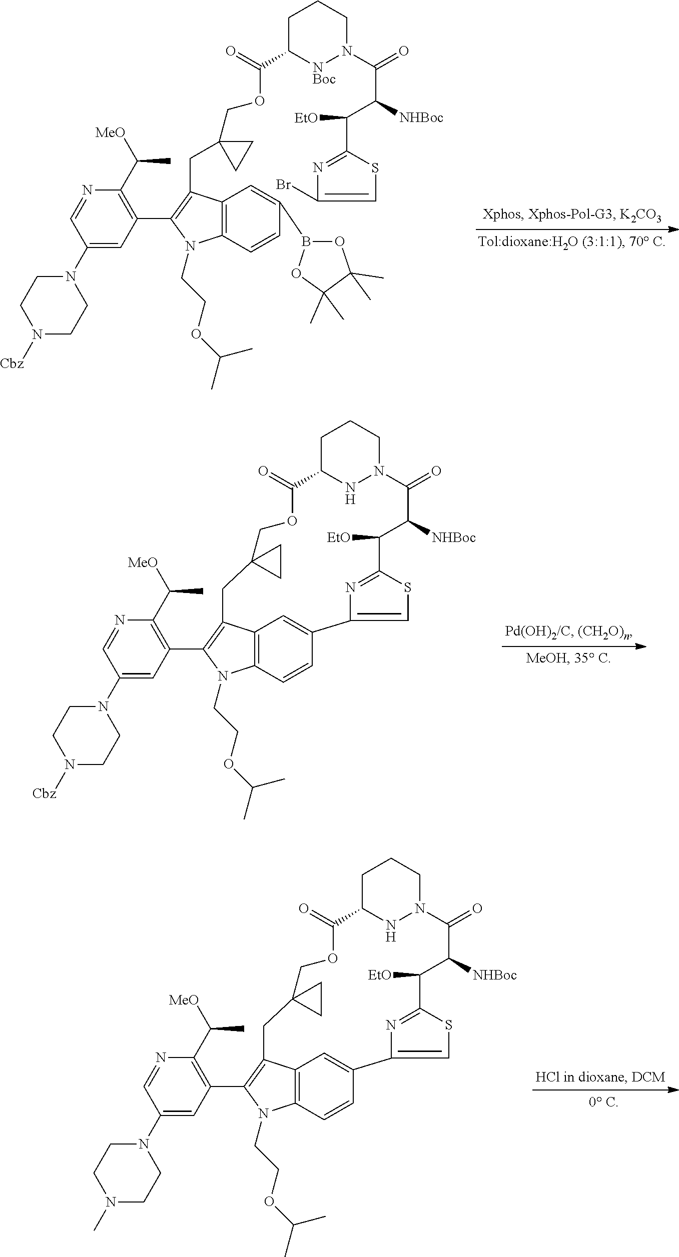 [SMILES: CCO[C@H](c1nc(Br)cs1)[C@H](NC(=O)OC(C)(C)C)C(=O)N1CCC[C@@H](C(=O)OCC2(Cc3c(-c4cc(N5CCN(C(=O)OCc6ccccc6)CC5)cnc4[C@H](C)OC)n(CCOC(C)C)c4ccc(B5OC(C)(C)C(C)(C)O5)cc34)CC2)N1C(=O)OC(C)(C)C ; CCO[C@@H]1c2nc(cs2)-c2ccc3c(c2)c(c(-c2cc(N4CCN(C(=O)OCc5ccccc5)CC4)cnc2[C@H](C)OC)n3CCOC(C)C)CC2(CC2)COC(=O)[C@@H]2CCCN(N2)C(=O)[C@H]1NC(=O)OC(C)(C)C ; CCO[C@@H]1c2nc(cs2)-c2ccc3c(c2)c(c(-c2cc(N4CCN(C)CC4)cnc2[C@H](C)OC)n3CCOC(C)C)CC2(CC2)COC(=O)[C@@H]2CCCN(N2)C(=O)[C@H]1NC(=O)OC(C)(C)C]

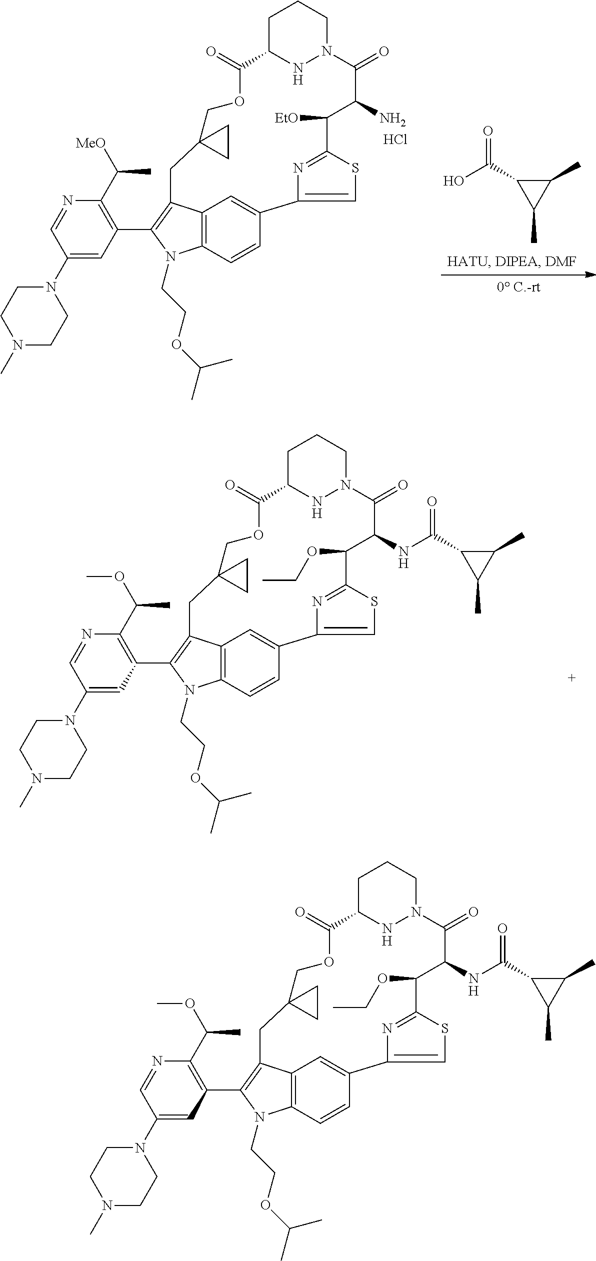

### Step 1.

To a mixture of 5-bromo-3-((1-(((tert-butyldiphenylsilyl)oxy) methyl)cyclopropyl)methyl)-2-iodo-1H-indole (6.1 g, 9.5 mmol) and benzyl(S)-4-(6-(1-methoxyethyl)-5-(4,4,5,5-tetramethyl-1,3,2-dioxaborolan-2-yl) pyridin-3-yl) piperazine-1-carboxylate (4.56 g, 9.5 mmol) in 1,4-dioxane (120 mL) and H₂O (20 mL) under an atmosphere of N₂ was added Pd(dppf)Cl₂ (1.04 g, 1.4 mmol) and K₃PO₄ (4.02 g, 18.9 mmol) in portions. The mixture was heated to 65° C. and stirred for 3 h, then extracted with EtOAc (3×100 mL). The combined organic layers were washed with brine (3×100 mL), dried over anhydrous Na₂SO₄ and filtered. The filtrate was concentrated under reduced pressure and the residue was purified by silica gel column chromatography to give benzyl(S)-4-(5-(5-bromo-3-((1-(((tert-butyldiphenylsilyl)oxy) methyl)cyclopropyl)methyl)-1H-indol-2-yl)-6-(1-methoxyethyl) pyridin-3-yl) piperazine-1-carboxylate (4.7 g, 57% yield) as a solid. LCMS (ESI): m/z [M+H]⁺ calc'd for C₄₉H₅₅BrN₄O₄Si 870.3; found 871.5.

### Step 2.

To a mixture of benzyl(S)-4-(5-(5-bromo-3-((1-(((tert-butyldiphenylsilyl)oxy) methyl)cyclopropyl)methyl)-1H-indol-2-yl)-6-(1-methoxyethyl) pyridin-3-yl) piperazine-1-carboxylate (4.6 g, 5.3 mmol) in DMF (50 mL) at 0° C. was added 2-isopropoxyethyl 4-methylbenzenesulfonate (2.04 g, 7.9 mmol) and Cs₂CO₃ (5.16 g, 15.8 mmol). The mixture was heated to 50° C. and stirred for 2 h, then diluted with H₂O and extracted with EtOAc (2×100 mL). The combined organic layers were washed with brine (3×100 mL), dried over anhydrous Na₂SO₄ and filtered. The filtrate was concentrated under reduced pressure and the residue was purified by reverse-phase silica gel column chromatography to give benzyl(S)-4-(5-(5-bromo-3-((1-(((tert-butyldiphenylsilyl)oxy) methyl)cyclopropyl)methyl)-1-(2-isopropoxyethyl)-1H-indol-2-yl)-6-(1-methoxyethyl) pyridin-3-yl) piperazine-1-carboxylate (4.5 g, 89% yield) as an oil. LCMS (ESI): m/z [M+H]⁺ calc'd for C₅₄H₆₅BrN₄O₅Si 956.4; found 957.2.

### Step 3.

To a mixture of benzyl(S)-4-(5-(5-bromo-3-((1-(((tert-butyldiphenylsilyl)oxy) methyl)cyclopropyl)methyl)-1-(2-isopropoxyethyl)-1H-indol-2-yl)-6-(1-methoxyethyl) pyridin-3-yl) piperazine-1-carboxylate (3.2 g, 3.3 mmol) in THF (32 mL) at 0° C. was added TBAF (13.1 g, 50.1 mmol). The mixture was warmed to room temperature and stirred for 16 h, then diluted with H₂O and extracted with EtOAc (2×100 mL). The combined organic layers were washed with brine (3×100 mL), dried over anhydrous Na₂SO₄ and filtered. The filtrate was concentrated under reduced pressure and the residue was purified by reverse-phase silica gel column chromatography to give benzyl(S)-4-(5-(5-bromo-3-((1-(hydroxymethyl)cyclopropyl)methyl)-1-(2-isopropoxyethyl)-1H-indol-2-yl)-6-(1-methoxyethyl) pyridin-3-yl) piperazine-1-carboxylate (1.84 g, 77% yield) as an oil. LCMS (ESI): m/z [M+H]⁺ calc'd for C₃₈H₄₇BrN₄O₅ 718.3; found 719.2.

### Step 4.

To a mixture of benzyl(S)-4-(5-(5-bromo-3-((1-(hydroxymethyl)cyclopropyl)methyl)-1-(2-isopropoxyethyl)-1H-indol-2-yl)-6-(1-methoxyethyl) pyridin-3-yl) piperazine-1-carboxylate (1.7 g, 2.4 mmol) in toluene (20 mL) at 0° C. under an atmosphere of Ar was added 4,4,4′, 4′, 5,5,5′, 5′-octamethyl-2,2′-bi (1,3,2-dioxaborolane) (1.80 g, 7.1 mmol), KOAc (580 mg, 5.9 mmol) and Pd(dppf)Cl₂ (346 mg, 0.47 mmol). The mixture was heated to 90° C. and stirred for 3 h, then diluted with H₂O and extracted with EtOAc (2×50 mL). The combined organic layers were washed with brine (2×50 mL), dried over anhydrous Na₂SO₄ and filtered. The filtrate was concentrated under reduced pressure and the residue was purified by reverse-phase silica gel column chromatography to give benzyl(S)-4-(5-(3-((1-(hydroxymethyl)cyclopropyl)methyl)-1-(2-isopropoxyethyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (1.4 g, 77% yield) as a solid. LCMS (ESI): m/z [M+H]⁺ calc'd for C₄₄H₅₉BN₄O₇ 766.5; found 767.4.

### Step 5.

To a mixture of benzyl(S)-4-(5-(3-((1-(hydroxymethyl)cyclopropyl)methyl)-1-(2-isopropoxyethyl)-5-(4,4,5,5-tetramethyl-1,3,2-dioxaborolan-2-yl)-1H-indol-2-yl)-6-(1-methoxyethyl) pyridin-3-yl) piperazine-1-carboxylate (1.4 g, 1.8 mmol) in DCM (20 mL) at 0° C. was added(S)-1,2-bis (tert-butoxycarbonyl) hexahydropyridazine-3-carboxylic acid (664 mg, 2.0 mmol), DCC (490 mg, 2.4 mmol) and DMAP (45 mg, 0.37 mmol). The mixture was warmed to room temperature and stirred for 16 h, then diluted with H₂O and extracted with EtOAc (2×100 mL). The combined organic layers were washed with brine (2×100 mL), dried over anhydrous Na₂SO₄ and filtered. The filtrate was concentrated under reduced pressure and the residue was purified by reverse-phase silica gel column to chromatography give 3-((1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl) 1,2-di-tert-butyl(S)-tetrahydropyridazine-1,2,3-tricarboxylate (1.2 g, 61% yield) as an oil. LCMS (ESI): m/z [M+H]⁺ calc'd for C₅₉H₈₃BN₆O₁₂ 1078.6; found 1079.5.

### Step 6.

A mixture of 3-((1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl) 1,2-di-tert-butyl(S)-tetrahydropyridazine-1,2,3-tricarboxylate (1.2 g, 1.1 mmol) in HCl in 1,4-dioxane (15 mL) at 0° C. was stirred for 2 h, then concentrated under reduced pressure to give (1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl(S)-hexahydropyridazine-3-carboxylate (1.25 g), which was used directly in the next step without further purification. LCMS (ESI): m/z [M+H]⁺ calc'd for C₄₉H₆₇BN₆O₈ 878.5; found 879.4.

### Step 7.

To a mixture of (1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl(S)-hexahydropyridazine-3-carboxylate (1.0 g, 1.1 mmol) in DMF (15 mL) at 0° C. was added DIPEA (1.47 g, 11.4 mmol) and (2S,3S)-3-(4-bromothiazol-2-yl)-2-((tert-butoxycarbonyl) amino)-3-ethoxypropanoic acid (675 mg, 1.7 mmol), followed by HATU (865 mg, 2.3 mmol). The mixture was stirred at 0° C. for 2 h, then diluted with H₂O and extracted with EtOAc (2×100 mL). The combined organic layers were washed with brine (2×100 mL), dried over anhydrous Na₂SO₄ and filtered. The filtrate was concentrated under reduced pressure and the residue was purified by preparative-TLC to give (1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl(S)-1-((2S,3S)-3-(4-bromothiazol-2-yl)-2-((tert-butoxycarbonyl) amino)-3-ethoxypropanoyl) hexahydropyridazine-3-carboxylate (730 mg, 51% yield) as an oil. LCMS (ESI): m/z [M+H]⁺ calc'd for C₆₂H₈₄BBrN₈O₁₂S 1254.5; found 1255.4.

### Step 8.

To a mixture of (1-((2-(5-(4-((benzyloxy) carbonyl) piperazin-1-yl)-2-((S)-1-methoxyethyl) pyridin-3-yl)-1-(2-isopropoxyethyl)-5-(4,4,5,5-tetramethyl-1,3,2-dioxaborolan-2-yl)-1H-indol-3-yl)methyl) cyclopropyl)methyl(S)-1-((2S,3S)-3-(4-bromothiazol-2-yl)-2-((tert-butoxycarbonyl) amino)-3-ethoxypropanoyl) hexahydropyridazine-3-carboxylate (700 mg, 0.56 mmol) in toluene (12 mL), 1,4-dioxane (4 mL) and H₂O (4 mL) at 0° C. under an atmosphere of Ar was added XPhos (53 mg, 0.11 mmol), K₃PO₄ (296 mg, 1.39 mmol) and XPho-Pd-G3 (47 mg, 0.06 mmol). The mixture was heated to 65° C. and stirred for 2 h, then diluted with H₂O and extracted with EtOAc (2×50 mL). The combined organic layers were washed with brine (2×50 mL), dried over anhydrous Na₂SO₄ and filtered. The filtrate was concentrated under reduced pressure and the residue was purified by preparative-TLC to give benzyl 4-(5-((3′S,3′S,4′S,Z)-4′-((tert-butoxycarbonyl) amino)-3′-ethoxy-1′-(2-isopropoxyethyl)-5′, 7′-dioxospiro[cyclopropane-1,10′-8-oxa-2 (4,2)-thiazola-1 (5,3)-indola-6 (1,3)-pyridazinacycloundecaphan]-2′-yl)-6-((S)-1-methoxyethyl) pyridin-3-yl) piperazine-1-carboxylate (160 mg, 27% yield) as an oil. LCMS (ESI): m/z [M+H]⁺ calc'd for C₅₆H₇₂N₈O₁₀S 1048.5; found 1049.3.

### Step 9.

To a mixture of benzyl 4-(5-((3′S,3′S,4′S,Z)-4′-((tert-butoxycarbonyl) amino)-3′-ethoxy-1′-(2-isopropoxyethyl)-5′, 7′-dioxospiro[cyclopropane-1,10′-8-oxa-2 (4,2)-thiazola-1 (5,3)-indola-6 (1,3)-pyridazinacycloundecaphan]-2′-yl)-6-((S)-1-methoxyethyl) pyridin-3-yl) piperazine-1-carboxylate (160 mg, 0.15 mmol) in MeOH (3 mL) at 0° C. was added methoxymethanol amine (34 mg, 0.76 mmol) and Pd (OH) 2/C (171 mg, 1.2 mmol). The mixture was placed under an atmosphere of H₂, heated to 35° C. and stirred for 4 h, then filtered, and the filter cake was washed with MeOH (2×50 mL). The filtrate was concentrated under reduced pressure to give tert-butyl ((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl) carbamate (100 mg), which was used directly in the next step without further purification. LCMS (ESI): m/z [M+H]⁺ calc'd for C₄₉H₆₈N₈O₈S 928.5; found 929.4.

### Step 10.

To a mixture of tert-butyl ((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl) carbamate (100 mg, 0.11 mmol) in DCM (1 mL) at 0° C. was added HCl in 1,4-dioxane (1 mL). The mixture was stirred at 0° C. for 2 h, then concentrated under reduced pressure to give (3′S,3′S,4′S,Z)-4′-amino-3′-ethoxy-1′-(2-isopropoxyethyl)-2′-(2-((S)-1-methoxyethyl)-5-(4-methylpiperazin-1-yl) pyridin-3-yl) spiro[cyclopropane-1,10′-8-oxa-2 (4,2)-thiazola-1 (5,3)-indola-6 (1,3)-pyridazinacycloundecaphane]-5′, 7′-dione (110 mg), which was used directly in the next step without further purification. LCMS (ESI): m/z [M+H]⁺ calc'd for C₄₄H₆₀N₈O₆S 828.4; found 829.4.

### Step 11.

To a mixture of (3′S,3′S,4′S,Z)-4′-amino-3′-ethoxy-1′-(2-isopropoxyethyl)-2′-(2-((S)-1-methoxyethyl)-5-(4-methylpiperazin-1-yl) pyridin-3-yl) spiro[cyclopropane-1,10′-8-oxa-2 (4,2)-thiazola-1 (5,3)-indola-6 (1,3)-pyridazinacycloundecaphane]-5′, 7′-dione (100 mg, 0.12 mmol) in DMF (2 mL) at 0° C. was added DIPEA (78 mg, 0.61 mmol), (1R,2R,3S)-2,3-dimethylcyclopropane-1-carboxylic acid (21 mg, 0.18 mmol) and HATU (92 mg, 0.24 mmol). The mixture was stirred at 0° C. for 2 h, then diluted with H₂O and extracted with EtOAc (2×30 mL). The combined organic layers were washed with brine (2×30 mL), dried over anhydrous Na₂SO₄ and filtered. The filtrate was concentrated under reduced pressure and the residue was purified by preparative-HPLC to give (1R,2R,3S)—N-((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl)-2,3-dimethylcyclopropane-1-carboxamide (7 mg, 6% yield) and (1R,2R,3S)—N-((3′S,3′S,4′S,Z)-3′-ethoxy-1′-(2-isopropoxyethyl)-2′-(2-((S)-1-methoxyethyl)-5-(4-methylpiperazin-1-yl) pyridin-3-yl)-5′, 7′-dioxospiro[cyclopropane-1,10′-8-oxa-2 (4,2)-thiazola-1 (5,3)-indola-6 (1,3)-pyridazinacycloundecaphan]-4′-yl)-2,3-dimethylcyclopropane-1-carboxamide (13 mg, 12% yield), both as solids. LCMS (ESI): m/z [M+H]⁺ calc'd for C₅₀H₆₈N₈O₇S 924.5; found 925.5; ¹H NMR (400 MHZ, DMSO-d₆) δ 8.50 (d, J=1.5 Hz, 1H), 8.42 (d, J=3.0 Hz, 1H), 7.91 (s, 1H), 7.71 (d, J=8.6 Hz, 1H), 7.64 (d, J=10.1 Hz, 1H), 7.58 (d, J=8.6 Hz, 1H), 7.25 (d, J=2.8 Hz, 1H), 6.55 (s, 1H), 5.77 (d, J=10.1 Hz, 1H), 5.04 (d, J=12.3 Hz, 1H), 4.92 (s, 1H), 4.37-4.28 (m, 1H), 4.22 (d, J=10.9 Hz, 1H), 4.18-4.08 (m, 3H), 3.98 (dd, J=12.2, 6.1 Hz, 3H), 3.65-3.58 (m, 6H), 3.36-3.20 (m, 6H), 3.17 (d, J=5.2 Hz, 2H), 2.98 (s, 3H), 2.45 (s, 6H), 2.39-2.30 (m, 3H), 2.10-1.89 (m, 3H), 1.86-1.66 (m, 3H), 1.51 (d, J=3.8 Hz, 2H), 1.36 (d, J=6.2 Hz, 3H), 1.24 (s, 1H), 1.16 (t, J=7.0 Hz, 5H), 1.08 (d, J=5.5 Hz, 3H), 1.04 (d, J=5.5 Hz, 3H), 0.91 (d, J=6.1 Hz, 3H), 0.86 (d, J=6.1 Hz, 4H) and LCMS (ESI): m/z [M+H]⁺ calc'd for C₅₀H₆₈N₈O₇S 924.5; found 925.5; ¹H NMR (400 MHZ, DMSO-d₆) δ 8.52 (s, 1H), 8.47 (d, J=3.1 Hz, 1H), 7.76-7.71 (m, 1H), 7.67 (d, J=10.2 Hz, 1H), 7.57 (d, J=8.5 Hz, 1H), 7.37 (d, J=3.2 Hz, 1H), 5.76 (d, J=10.1 Hz, 1H), 5.03 (d, J=12.3 Hz, 1H), 4.92 (s, 1H), 4.15 (dd, J=27.1, 12.3 Hz, 1H), 3.83 (d, J=6.2 Hz, 3H), 3.54 (dd, J=17.3, 6.9 Hz, 1H), 3.44 (d, J=6.7 Hz, 3H), 3.34-3.26 (m, 9H), 2.92 (s, 3H), 2.47-2.41 (m, 7H), 2.37-2.31 (m, 3H), 2.23 (s, 1H), 1.94-1.77 (m, 3H), 1.55 (s, 2H), 1.24 (d, J=6.4 Hz, 4H), 1.15 (t, J=6.9 Hz, 5H), 1.09 (d, J=5.7 Hz, 3H), 1.04 (d, J=5.7 Hz, 3H), 0.95 (d, J=6.1 Hz, 3H), 0.86 (d, J=6.1 Hz, 3H), 0.50-0.42 (m, 1H), 0.33 (s, 2H), 0.14-0.06 (m, 1H).

### Example A483. Synthesis of (1r,2R,3S)—N-((6³S,3S,4S,Z)-12-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

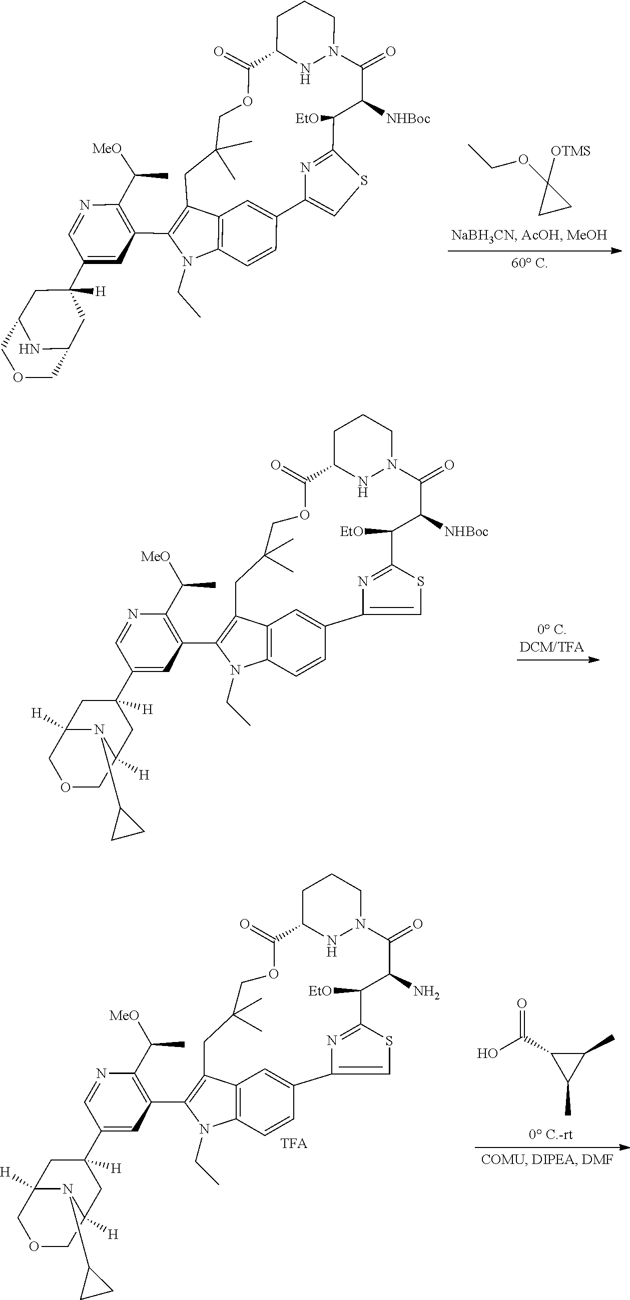

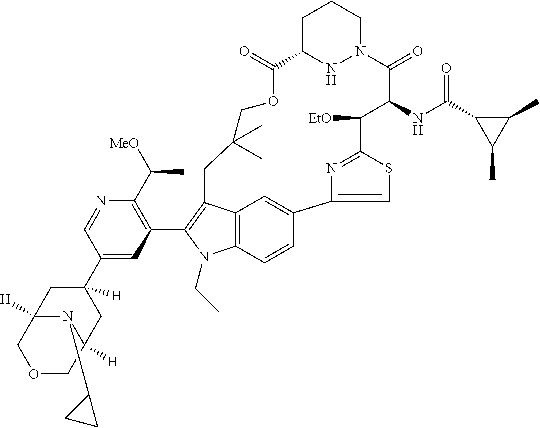

### Step 1.

To a mixture of tert-butyl ((6³S,3S,4S,Z)-12-(5-((1R,5S,7s)-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (50% purity; 500 mg, 0.28 mmol) and (1-ethoxycyclopropoxy) trimethylsilane (968 mg, 5.6 mmol) in MeOH (2 mL) at 0° C. was added AcOH (83 mg, 1.39 mmol) and NaBH₃CN (87 mg, 1.39 mmol) in portions. The mixture was warmed to 60° C. and stirred for 2 h, then concentrated under reduced pressure. The residue purified by silica gel column chromatography to give tert-butyl ((6³S,3S,4S,Z)-12-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (80 mg, 30% yield) as a solid. LCMS (ESI): m/z [M+H]⁺ calc'd for C₅₁H₆₉N₇O₈S 939.5; found 940.6.

### Step 2.

A mixture of tert-butyl ((6³S,3S,4S,Z)-12-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (80 mg, 0.09 mmol) and TFA (0.4 mL) in DCM (2 mL) at 0° C. was stirred for 1 h, then concentrated under reduced pressure to give (6³S,3S,4S,Z)-4-amino-12-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione as a TFA salt (180 mg) as an oil. LCMS (ESI): m/z [M+H]⁺ calc'd for C₄₆H₆₁N₇O₆S 839.4; found 840.4.

### Step 3.

To a mixture of (6³S,3S,4S,Z)-4-amino-12-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (180 mg, 0.21 mmol) and (1R,2R,3S)-2,3-dimethylcyclopropane-1-carboxylic acid (49 mg, 0.43 mmol) in DMF (3 mL) at 0° C. was added DIPEA (277 mg, 2.14 mmol) and COMU (184 mg, 0.43 mmol) in portions. The mixture was warmed to room temperature and stirred for 2 h. The residue was purified by preparative-HPLC to give (1r,2R,3S)—N-((6³S,3S,4S,Z)-1²-(5-((1R,5S,7s)-9-cyclopropyl-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (23 mg, 11% yield) as a solid. LCMS (ESI): m/z [M+H]⁺ calc'd for C₅₂H₆₉N₇O₇S 935.5; found 936.4; ¹H NMR (300 MHZ, DMSO-d₆) δ 8.74 (d, J=2.2 Hz, 1H), 8.56 (s, 1H), 7.98 (s, 1H), 7.81 (d, J=8.6 Hz, 1H), 7.74-7.56 (m, 3H), 5.94 (d, J=9.7 Hz, 1H), 5.23 (d, J=12.4 Hz, 1H), 5.00 (s, 1H), 4.42-4.08 (m, 5H), 3.92 (d, J=10.9 Hz, 2H), 3.90-3.65 (m, 6H) 3.57-3.46 (m, 2H), 3.26 (s, 4H), 3.02 (d, J=10.1 Hz, 2H), 2.89 (m, 2H), 2.67 (m, 1H), 2.35 (s, 2H), 2.13 (d, J=10.5 Hz, 1H), 1.85 (d, J=13.6 Hz, 3H), 1.59 (s, 2H), 1.43 (d, J=6.1 Hz, 3H), 1.42-1.30 (m, 2H), 1.28-1.08 (m, 12H), 1.10-0.98 (m, 7H), 0.55 (d, J=6.0 Hz, 2H), 0.45 (s, 2H), 0.39 (s, 2H).

### Example A484. Synthesis of (6³S,3S,4S,Z)-4-amino-3-ethoxy-1¹-ethyl-1²-(2-((S)-1-methoxyethyl)-5-((1R,5S,7s)-9-(oxetan-3-yl)-3-oxa-9-azabicyclo[3.3.1]nonan-7-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione

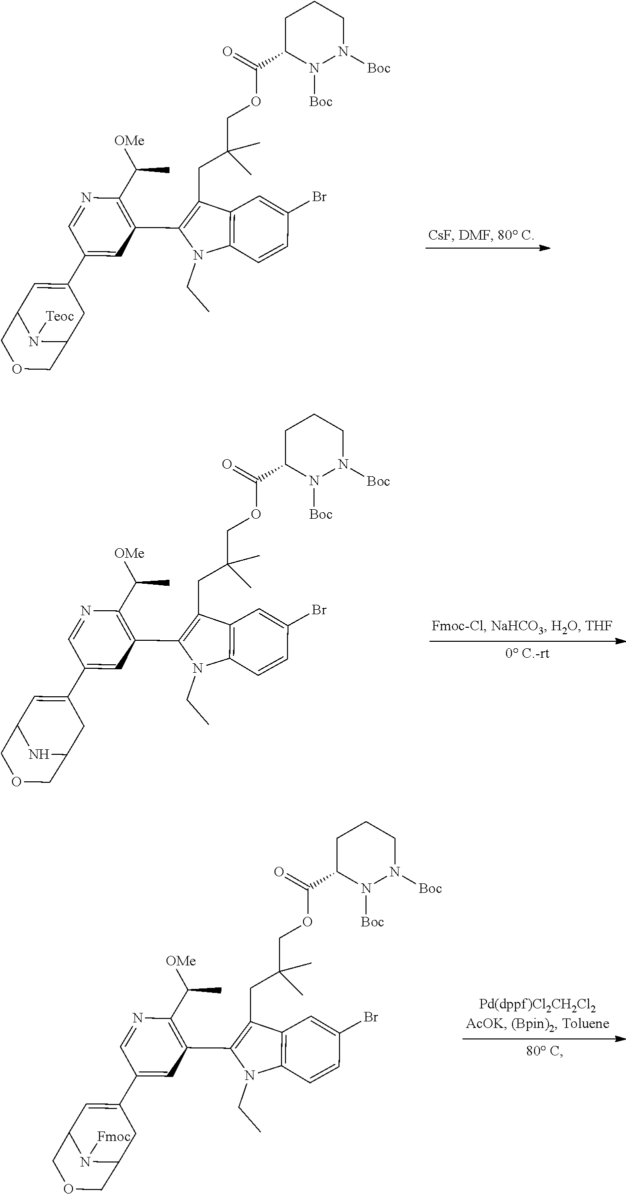

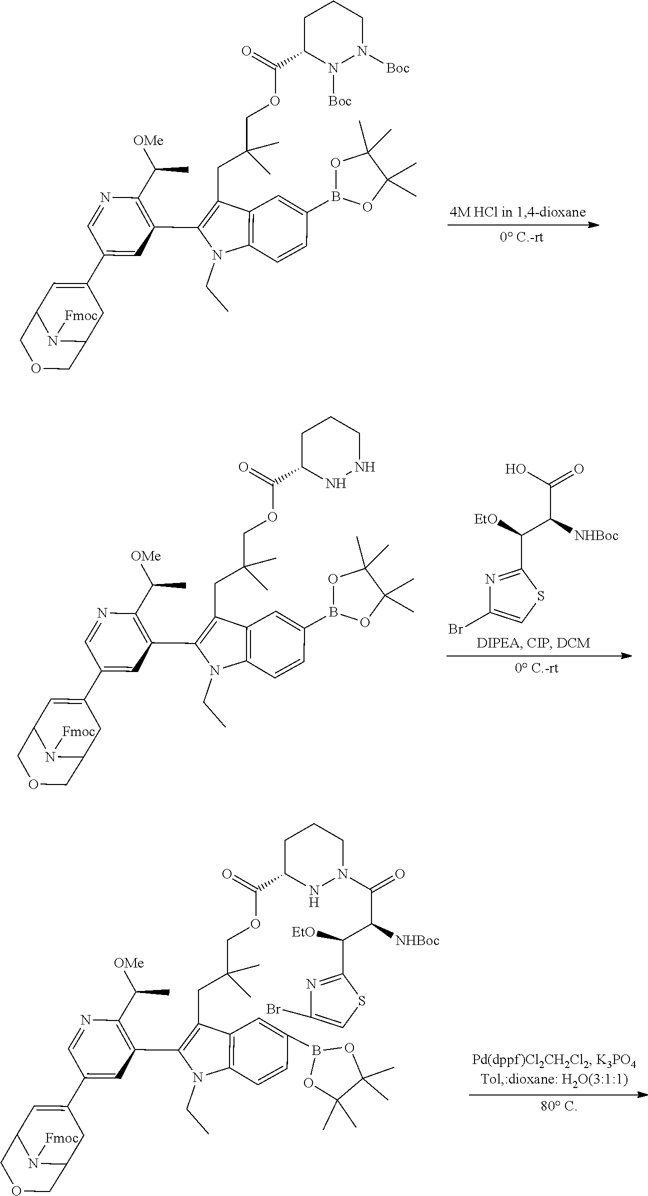

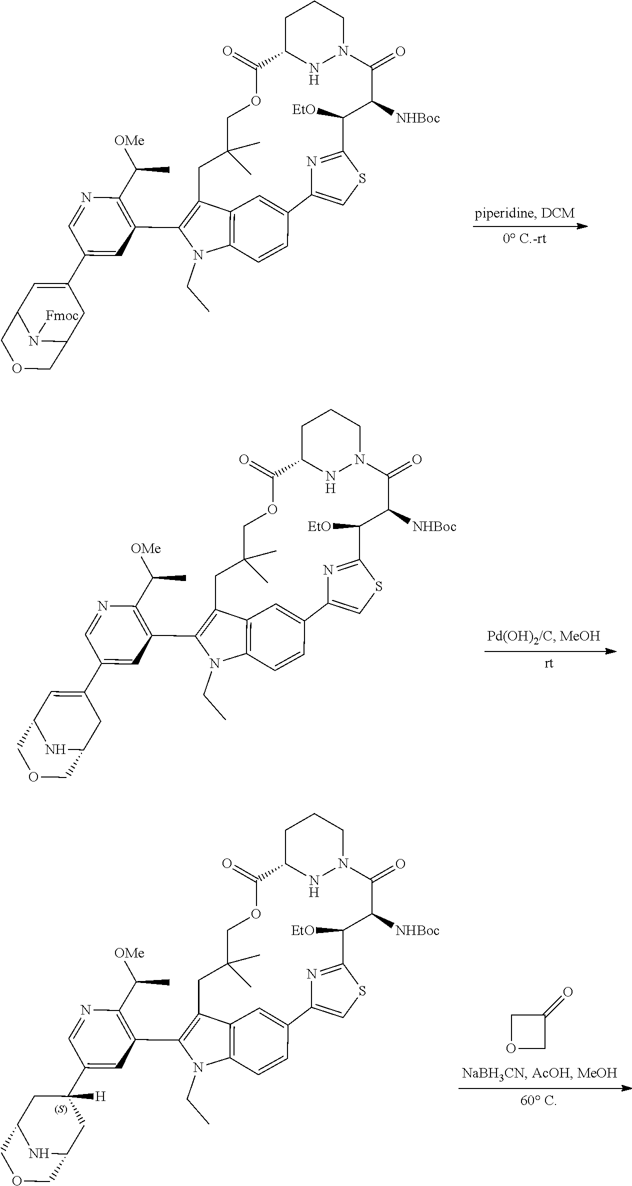

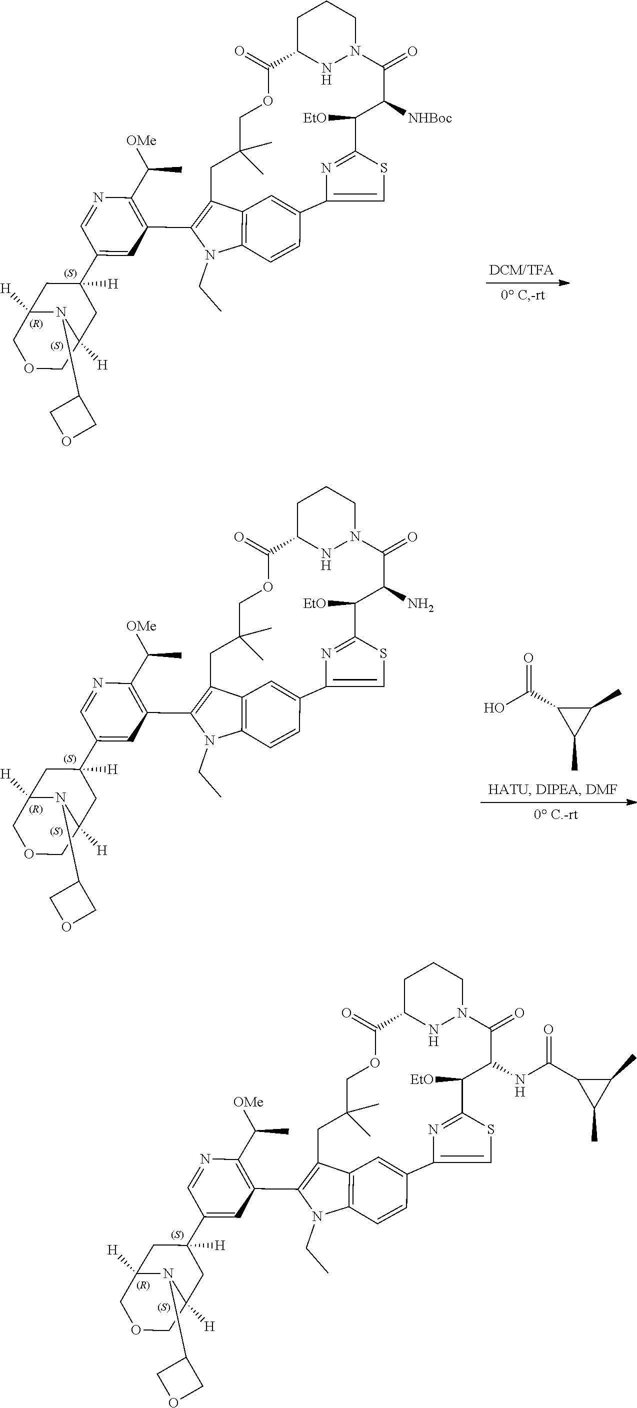

### Step 1.

A mixture of 3-(3-(5-bromo-1-ethyl-2-(2-((S)-1-methoxyethyl)-5-(9-((2,2,2-trichloroethoxy) carbonyl)-3-oxa-9-azabicyclo[3.3.1] non-6-en-7-yl) pyridin-3-yl)-1H-indol-3-yl)-2,2-dimethylpropyl)-1,2-di-tert-butyl-(3S)-tetrahydropyridazine-1,2,3-tricarboxylate (6.57 g, 6.22 mmol) and CsF (4.72 g, 31.1 mmol) in DMF (50 mL) was heated to 80° C. and stirred for 2 h, then diluted with H₂O (3×200 mL) and extracted with EtOAc (200 mL). The organic layer was concentrated under reduced pressure to give 3-(3-(2-(5-(3-oxa-9-azabicyclo[3.3.1] non-6-en-7-yl)-2-((S)-1-methoxyethyl) pyridin-3-yl)-5-bromo-1-ethyl-1H-indol-3-yl)-2,2-dimethylpropyl)-1,2-di-tert-butyl-(3S)-tetrahydropyridazine-1,2,3-tricarboxylate (5.83 g) as a solid. LCMS (ESI): m/z [M+H]⁺ calc'd for C₄₅H₆₂BrN₅O₈ 879.4 & 881.4; found 880.1 & 882.1.

### Step 2.

To a mixture of 3-(3-(2-(5-(3-oxa-9-azabicyclo[3.3.1] non-6-en-7-yl)-2-((S)-1-methoxyethyl) pyridin-3-yl)-5-bromo-1-ethyl-1H-indol-3-yl)-2,2-dimethylpropyl)-1,2-di-tert-butyl-(3S)-tetrahydropyridazine-1,2,3-tricarboxylate (5.83 g, 6.62 mmol) and NaHCO₃ (2.78 g, 33.1 mmol) in H₂O (20 mL) and THF (20 mL) at 0° C. was added 9H-fluoren-9-ylmethyl chloroformate (2.57 g, 9.93 mmol) in portions. The mixture was warmed to room temperature and stirred overnight, then extracted with EtOAc (3×50 mL). The combined organic layers were dried over anhydrous Na₂SO₄, filtered, the filtrate was concentrated under reduced pressure and the residue was purified by silica gel column chromatography to give 3-(3-(2-(5-(9-(((9H-fluoren-9-yl) methoxy) carbonyl)-3-oxa-9-azabicyclo[3.3.1] non-6-en-7-yl)-2-((S)-1-methoxyethyl) pyridin-3-yl)-5-bromo-1-ethyl-1H-indol-3-yl)-2,2-dimethylpropyl) 1,2-di-tert-butyl (3S)-tetrahydropyridazine-1,2,3-tricarboxylate (6.63 g, 90% yield) as a solid. LCMS (ESI): m/z [M+H]⁺ calc'd for C₆₀H₇₂BrN₅O₁₀ 1101.5 & 1103.4; found 1102.4 & 1104.4.

### Step 3.

To a mixture of 3-(3-(2-(5-(9-(((9H-fluoren-9-yl) methoxy) carbonyl)-3-oxa-9-azabicyclo[3.3.1] non-6-en-7-yl)-2-((S)-1-methoxyethyl) pyridin-3-yl)-5-bromo-1-ethyl-1H-indol-3-yl)-2,2-dimethylpropyl) 1,2-di-tert-butyl (3S)-tetrahydropyridazine-1,2,3-tricarboxylate (6.4 g, 5.8 mmol) and bis (pinacolato)diboron (2.21 g, 8.7 mmol) in toluene (25 mL) under an atmosphere of N₂ was added AcOK (1.42 g, 14.5 mmol) and Pd(dppf)Cl₂CH₂Cl₂ (0.47 g, 0.58 mmol) in portions. The mixture was heated to 80° C. and stirred for 3 h, then concentrated under reduced pressure and the residue was purified by silica gel column chromatography to give 3-(3-(2-(5-(9-(((9H-fluoren-9-yl) methoxy) carbonyl)-3-oxa-9-azabicyclo[3.3.1] non-6-en-7-yl)-2-((S)-1-methoxyethyl) pyridin-3-yl)-1-ethyl-5-(4,4,5,5-tetramethyl-1,3,2-dioxaborolan-2-yl)-1H-indol-3-yl)-2,2-dimethylpropyl) 1,2-di-tert-butyl (3S)-tetrahydropyridazine-1,2,3-tricarboxylate (4.9 g, 73% yield) as a solid. LCMS (ESI): m/z [M+H]⁺ calc'd for C₆₆H₈₄BN₅O₁₂ 1149.6; found 1150.8.

### Step 4.

To 3-(3-(2-(5-(9-(((9H-fluoren-9-yl) methoxy) carbonyl)-3-oxa-9-azabicyclo[3.3.1] non-6-en-7-yl)-2-((S)-1-methoxyethyl) pyridin-3-yl)-1-ethyl-5-(4,4,5,5-tetramethyl-1,3,2-dioxaborolan-2-yl)-1H-indol-3-yl)-2,2-dimethylpropyl) 1,2-di-tert-butyl (3S)-tetrahydropyridazine-1,2,3-tricarboxylate (4.9 g, 4.3 mmol) was added HCl in 1,4-dioxane (15 mL) at 0° C. The mixture was warmed to room temperature and stirred for 5 h, then concentrated under reduced pressure to give (9H-fluoren-9-yl)methyl-7-(5-(1-ethyl-3-(3-(((S)-hexahydropyridazine-3-carbonyl)oxy)-2,2-dimethylpropyl)-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1] non-6-ene-9-carboxylate (4.9 g) as a solid, that was used directly in the next step without further purification. LCMS (ESI): m/z [M+H]⁺ calc'd for C₅₆H₆₈BN₅O₈ 949.5; found 950.5.

### Step 5.

To a mixture of (9H-fluoren-9-yl)methyl-7-(5-(1-ethyl-3-(3-(((S)-hexahydropyridazine-3-carbonyl)oxy)-2,2-dimethylpropyl)-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1] non-6-ene-9-carboxylate (4.8 g, 5.1 mmol) and (2S,3S)-3-(4-bromo-1,3-thiazol-2-yl)-2-[(tert-butoxycarbonyl) amino]-3-ethoxypropanoic acid (3.00 g, 7.6 mmol) in DCM (30 mL) at 0° C. was added DIPEA (6.53 g, 50.5 mmol) and CIP (4.22 g, 15.2 mmol) in portions. The mixture was warmed to room temperature and stirred for 3 h, then concentrated under reduced pressure and the residue was purified by silica gel column chromatography to give (9H-fluoren-9-yl) methyl 7-(5-(3-(3-(((S)-1-((2S,3S)-3-(4-bromothiazol-2-yl)-2-((tert-butoxycarbonyl) amino)-3-ethoxypropanoyl) hexahydropyridazine-3-carbonyl)oxy)-2,2-dimethylpropyl)-1-ethyl-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1] non-6-ene-9-carboxylate (5.5 g, 82% yield) as a solid. LCMS (ESI): m/z [M+H]⁺ calc'd for C₆₉H₈₅BBrN₇O₁₂S 1325.5 & 1327.5; found 1326.5 & 1328.5.

### Step 6.

To a mixture of (9H-fluoren-9-yl) methyl 7-(5-(3-(3-(((S)-1-((2S,3S)-3-(4-bromothiazol-2-yl)-2-((tert-butoxycarbonyl) amino)-3-ethoxypropanoyl) hexahydropyridazine-3-carbonyl)oxy)-2,2-dimethylpropyl)-1-ethyl-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1] non-6-ene-9-carboxylate (5.5 g, 4.1 mmol) and K₃PO₄ (2.20 g, 10.4 mmol) in toluene (12 mL), dioxane (4 mL) and H₂O (4 mL) under an atmosphere of N₂ was added Pd(dppf)Cl₂·DCM (0.34 g, 0.41 mmol) in portions. The mixture was heated to 80° C. and stirred for 3 h, then filtered and the filter cake was washed with EtOAc (3×10 mL) and H₂O (10 mL). The filtrate was partioned and the aqueous layer was extracted with EtOAc (3×20 mL). The combined organic layers were dried over anhydrous Na₂SO₄, filtered, the filtrate was concentrated under reduced pressure and the residue was purified by silica gel column chromatography to give (9H-fluoren-9-yl)methyl-7-(5-((6³S,3S,4S,Z)-4-((tert-butoxycarbonyl) amino)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 12-yl)-6-((S)-1-methoxyethyl) pyridin-3-yl)-3-oxa-9-azabicyclo[3.3.1] non-6-ene-9-carboxylate (2.3 g, 49% yield) as a solid. LCMS (ESI): m/z [M+H]⁺ calc'd for C₆₃H₇₃N₇O₁₀S 1119.5; found 1120.3.

### Step 7.

To a mixture of (9H-fluoren-9-yl)methyl-7-(5-((6³S,3S,4S,Z)-4-((tert-butoxycarbonyl) amino)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-3-oxa-9-azabicyclo[3.3.1] non-6-ene-9-carboxylate (2.3 g, 2.1 mmol) and piperidine (0.87 g, 10.3 mmol) in DCM (10 mL) was stirred at room temperature for 3 h, then concentrated under reduced pressure to give tert-butyl ((6³S,3S,4S,Z)-12-(5-((1R,5S)-3-oxa-9-azabicyclo[3.3.1] non-6-en-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate (2.3 g) as an oil. LCMS (ESI): m/z [M+H]⁺ calc'd for C₄₈H₆₃N₇O₈S 897.5; found 898.9.

### Step 8.

A mixture of tert-butyl ((6³S,3S,4S,Z)-12-(5-((1R,5S)-3-oxa-9-azabicyclo[3.3.1] non-6-en-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (2.3 g, 2.6 mmol) and Pd (OH) 2, 20% on carbon (2.30 g, 3.3 mmol) in MeOH (10 mL) was hydrogenated (balloon) at room temperature for 2 h. The mixture was filtered through a pad of Celite and the filtrate was concentrated under reduced pressure to give tert-butyl ((6³S,3S,4S,Z)-1²-(5-((1R,5S,7s)-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (2.3 g) as an oil, which was used directly in the next step without further purification. LCMS (ESI): m/z [M+H]⁺ calc'd for C₄₈H₆₅N₇O₈S 899.5; found 901.0.

### Step 9.

To a mixture of tert-butyl ((6³S,3S,4S,Z)-12-(5-((1R,5S,7s)-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (480 mg, 0.53 mmol) and 3-oxetanone (154 mg, 2.1 mmol) in MeOH (4 mL) at 0° C. was added AcOH (320 mg, 5.3 mmol) dropwise. The mixture was warmed to room temperature and stirred for 30 min, then the mixture was re-cooled to 0° C. and NaBH₃CN (101 mg, 1.6 mmol) was added in portions. The mixture was heated to 60° C. and stirred for 2 h, then concentrated under reduced pressure and the residue was purified by silica gel column chromatography to give tert-butyl ((6³S,3S,4S,Z)-3-ethoxy-1¹-ethyl-1²-(2-((S)-1-methoxyethyl)-5-((1R,5S,7s)-9-(oxetan-3-yl)-3-oxa-9-azabicyclo[3.3.1]nonan-7-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (200 mg, 39% yield) as a solid. LCMS (ESI): m/z [M+H]⁺ calc'd for C₅₁H₆₉N₇O₉S 955.5; found 956.7.

### Step 10.

To a mixture of tert-butyl ((6³S,3S,4S,Z)-3-ethoxy-1¹-ethyl-1²-(2-((S)-1-methoxyethyl)-5-((1R,5S,7s)-9-(oxetan-3-yl)-3-oxa-9-azabicyclo[3.3.1]nonan-7-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (200 mg, 0.21 mmol) in DCM (2 mL) at 0° C. was added TFA (0.4 mL) dropwise. The mixture was warmed to room temperature and stirred for 1 h, then concentrated under reduced pressure to give (6³S,3S,4S,Z)-4-amino-3-ethoxy-1¹-ethyl-1²-(2-((S)-1-methoxyethyl)-5-((1R,5S,7s)-9-(oxetan-3-yl)-3-oxa-9-azabicyclo[3.3.1]nonan-7-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (220 mg) as a solid. LCMS (ESI): m/z [M+H]⁺ calc'd for C₄₆H₆₁N₇O₇S 855.4; found 856.6.

### Step 11.

To a mixture of (6³S,3S,4S,Z)-4-amino-3-ethoxy-1¹-ethyl-1²-(2-((S)-1-methoxyethyl)-5-((1R,5S,7s)-9-(oxetan-3-yl)-3-oxa-9-azabicyclo [3.3.1]nonan-7-yl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (220 mg, 0.26 mmol) and (1r,2R,3S)-2,3-dimethylcyclopropane-1-carboxylic acid (59 mg, 0.51 mmol) in DMF (3 mL) at 0° C. under an atmosphere of N₂ was added DIPEA (332 mg, 2.57 mmol) and HATU (293 mg, 0.77 mmol) in portions. The mixture was warmed to room temperature and stirred for 2 h, then purified by preparative-HPLC to give (1r,2R,3S)—N-((6³S,3S,4S,Z)-3-ethoxy-1¹-ethyl-1²-(2-((S)-1-methoxyethyl)-5-((1R,5S,7s)-9-(oxetan-3-yl)-3-oxa-9-azabicyclo[3.3.1]nonan-7-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide (67 mg, 27% yield) as a solid. LCMS (ESI): m/z [M+H]⁺ calc'd for C₅₂H₆₉N₇O₈S 951.5; found 952.5; ¹H NMR (400 MHZ, DMSO-d₆) δ 8.79 (s, 1H), 8.50 (d, J=1.6 Hz, 1H), 7.92 (s, 1H), 7.79-7.72 (m, 2H), 7.65 (d, J=9.9 Hz, 1H), 7.58 (d, J=8.7 Hz, 1H), 5.86 (d, J=9.9 Hz, 1H), 5.15 (d, J=12.3 Hz, 2H), 4.91 (s, 1H), 4.74 (s, 4H), 4.37-4.20 (m, 3H), 4.18-4.05 (m, 3H), 3.92 (s, 3H), 3.57 (d, J=3.5 Hz, 5H), 3.51-3.44 (m, 7H), 3.22 (s, 3H), 2.90-2.74 (m, 2H), 2.55 (s, 2H), 2.39 (s, 2H), 2.06 (d, J=12.3 Hz, 1H), 1.78 (d, J=29.9 Hz, 2H), 1.56-1.47 (m, 2H), 1.37 (d, J=6.1 Hz, 3H), 1.16 (t, J=7.0 Hz, 5H), 1.11-0.94 (m, 7H), 0.93-0.77 (m, 6H), 0.36 (s, 3H).

### Example A496. Synthesis of (1S,2S)—N-((6³S,3S,4S,Z)-3-(dimethylamino)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methylcyclopropane-1-carboxamide

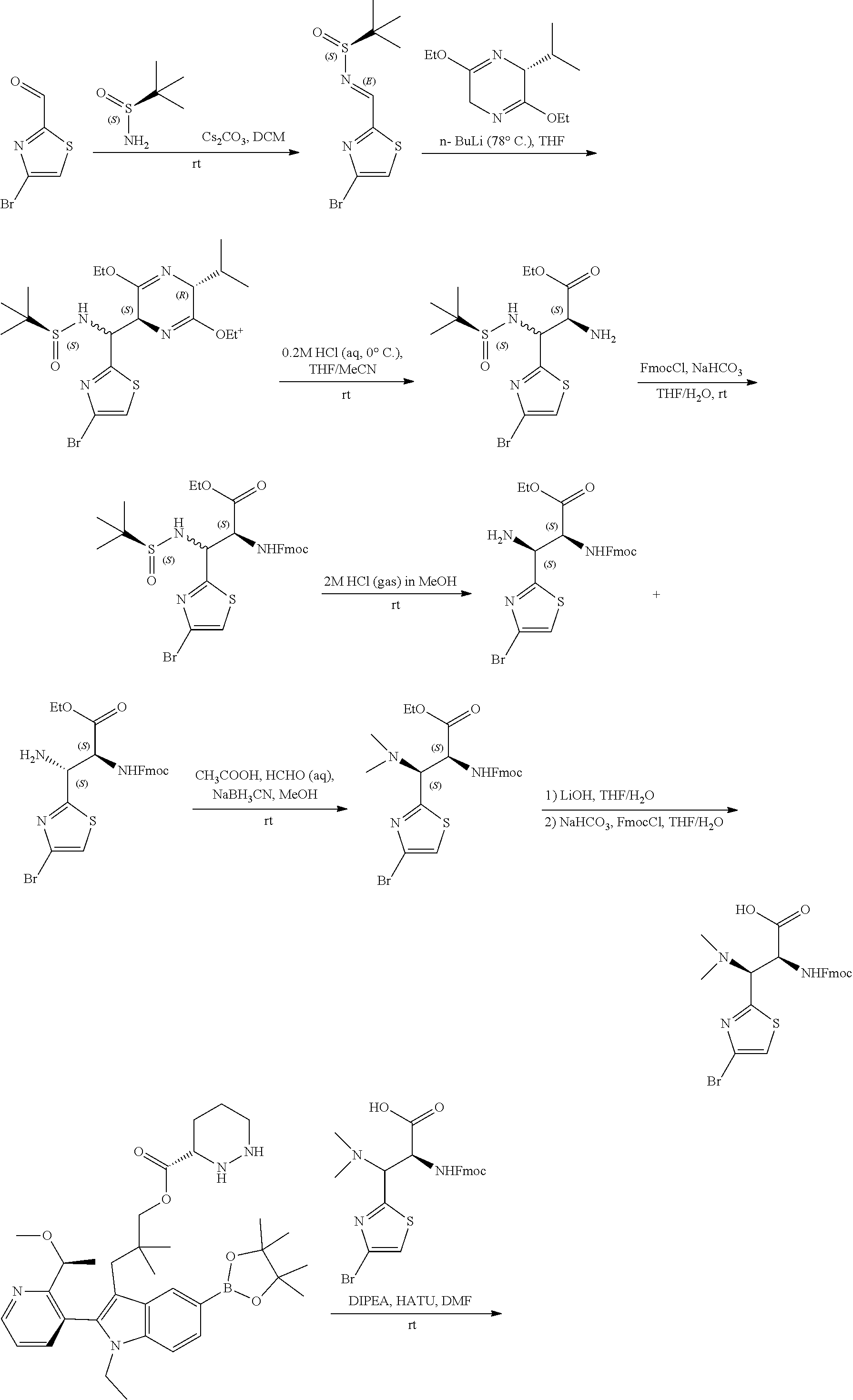

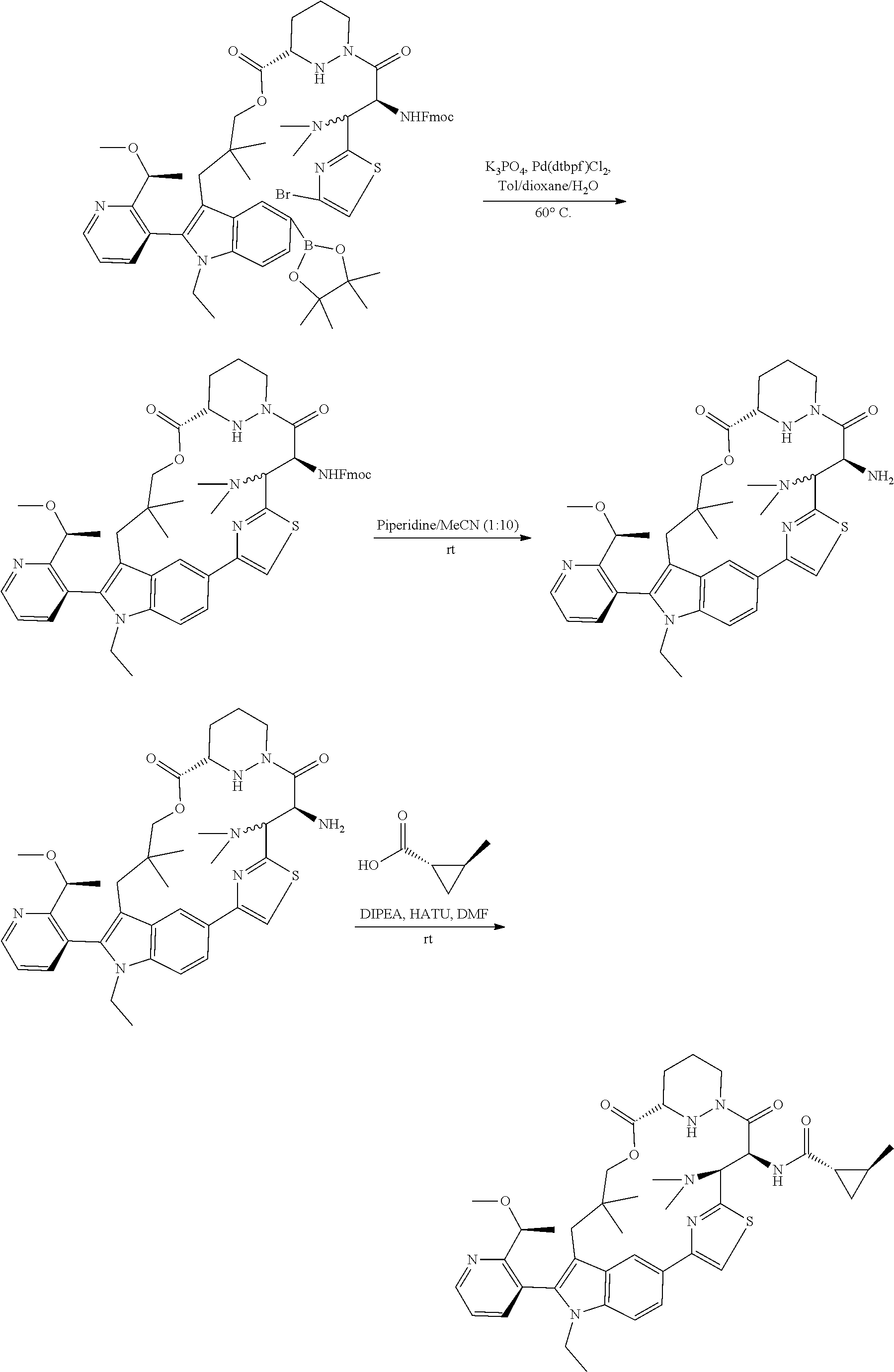

### Step 1.

To a mixture of 4-bromothiazole-2-carbaldehyde (15.0 g, 78.1 mmol) and(S)-2-methylpropane-2-sulfinamide (9.47 g, 78.1 mmol) in DCM was added Cs₂CO₃ (50.90 g, 156.2 mmol) in portions. The mixture was stirred at room temperature for 2 h, then filtered and the filter cake was washed with DCM (3×20 mL). The filtrate was concentrated under reduced pressure and the residue was purified by silica gel column chromatography to give (S,E)-N-((4-bromothiazol-2-yl)methylene)-2-methylpropane-2-sulfinamide (26 g, 97% yield) as an oil. LCMS (ESI): m/z [M+H]⁺ calc'd for C₈H₁₁BrN₂OS₂ 294.0; found 294.8.

### Step 2.

To a mixture of (R)-3,6-diethoxy-2-isopropyl-2,5-dihydropyrazine (20.57 g, 96.9 mmol) in THF at −78° C. under an atmosphere of N₂ was treated with n-BuLi in hexanes (20.5 mL, 105.7 mmol). The mixture was stirred at −78° C. for 30 min, then (S,E)-N-((4-bromothiazol-2-yl)methylene)-2-methylpropane-2-sulfinamide (26.0 g, 88.1 mmol) was added dropwise. The mixture was stirred at-78° C. for 2 h, then warmed to 0° C. and quenched with saturated NaHCO₃. The aqueous layer was extracted with EtOAc (3×500 mL), the combined organic layers were concentrated under reduced pressure and the residue was purified by silica gel column chromatography to give (S)—N—((S)-(4-bromothiazol-2-yl) ((2S,5R)-3,6-diethoxy-5-isopropyl-2,5-dihydropyrazin-2-yl)methyl)-2-methylpropane-2-sulfinamide (32.0 g, 72% yield) as an oil. LCMS (ESI): m/z [M+H]⁺ calc'd for C₁₉H₃₁BrN₄O₃S₂ 506.1; found 507.0.

### Step 3.

To a mixture of(S)—N—((S)-(4-bromothiazol-2-yl) ((2S,5R)-3,6-diethoxy-5-isopropyl-2,5-dihydropyrazin-2-yl)methyl)-2-methylpropane-2-sulfinamide (32.0 g, 63.0 mmol) in THF (1 L) and MECN (640 mL) at 0° C. was added 0.2M HCl (790 mL) dropwise. The mixture was warmed to room temperature and stirred overnight, then quenched by the addition of saturated NaHCO₃ and extracted with EtOAc (3×500 mL). The combined organic layers were concentrated under reduced pressure and the residue was purified by silica gel column chromatography to give ethyl (2S,3S)-2-amino-3-(4-bromothiazol-2-yl)-3-(((S)-tert-butylsulfinyl) amino) propanoate (18.0 g, 72% yield) as an oil. LCMS (ESI): m/z [M+H]⁺ calc'd for C₁₂H₂₀BrN₃O₃S₂ 397.0; found 398.1.

### Step 4.

To a mixture of ethyl (2S,3S)-2-amino-3-(4-bromothiazol-2-yl)-3-(((S)-tert-butylsulfinyl) amino) propanoate (15.0 g, 37.7 mmol) and NaHCO₃ (15.82 g, 188.3 mmol) in THF (100 mL) and H₂O (100 mL) was added FmocCl (11.69 g, 45.2 mmol) in portions. The mixture was stirred at room temperature for 2 h, then washed with H₂O (3×100 mL). The aqueous layer was extracted with EtOAc (3×100 mL), the combined organic layers were concentrated under reduced pressure and the residue was purified by silica gel column chromatography to give ethyl (2S,3S)-2-((((9H-fluoren-9-yl) methoxy) carbonyl) amino)-3-(4-bromothiazol-2-yl)-3-(((S)-tert-butylsulfinyl) amino) propanoate (20.0 g, 86% yield) as an oil. LCMS (ESI): m/z [M+H]⁺ calc'd for C₂₇H₃₀BrN₃O₅S₂ 619.1; found 620.0.

### Step 5.

A mixture of ethyl (2S,3S)-2-((((9H-fluoren-9-yl) methoxy) carbonyl) amino)-3-(4-bromothiazol-2-yl)-3-(((S)-tert-butylsulfinyl) amino) propanoate (20.0 g, 32.2 mmol) and 4M HCl in MeOH (150 mL) was stirred at room temperature for 2 h. The mixture was concentrated under reduced pressure and the residue was purified by reverse-phase silica gel column chromatography to give ethyl (2S,3S)-2-((((9H-fluoren-9-yl) methoxy) carbonyl) amino)-3-amino-3-(4-bromothiazol-2-yl) propanoate (7.5 g, 45% yield) as an oil. LCMS (ESI): m/z [M+H]⁺ calc'd for C₂₃H₂₂BrN₃O₄S 515.1; found 516.0.

### Step 6.

To a mixture of ethyl (2S,3S)-2-((((9H-fluoren-9-yl) methoxy) carbonyl) amino)-3-amino-3-(4-bromothiazol-2-yl) propanoate (1.2 g, 2.3 mmol) and AcOH (419 mg, 7.0 mmol) in MeOH (20 mL) was added HCHO, 37% aqueous solution (419 mg, 13.9 mmol) and NaBH₃CN (730 mg, 11.6 mmol) in portions. The mixture was stirred at room temperature for 2 h, then diluted with H₂O and extracted with EtOAc (3×200 mL). The combined organic layers were concentrated under reduced pressure and the residue was purified by silica gel column chromatography to give ethyl (2S,3S)-2-((((9H-fluoren-9-yl) methoxy) carbonyl) amino)-3-(4-bromothiazol-2-yl)-3-(dimethylamino) propanoate (990 mg, 78% yield) as an oil. LCMS (ESI): m/z [M+H]⁺ calc'd for C₂₅H₂₆BrN₃O₄S 543.1; found 543.8.

### Step 7.

A mixture of ethyl (2S,3S)-2-((((9H-fluoren-9-yl) methoxy) carbonyl) amino)-3-(4-bromothiazol-2-yl)-3-(dimethylamino) propanoate (990 mg, 1.8 mmol) and LiOH·H₂O (174 mg, 7.3 mmol) in THF (50 mL) and H₂O (50 mL) was stirred at room temperature for 1 h, then acidified to PH ˜5 with 1M HCl. The mixture was used directly in the next step without further purification.

### Step 8.

To the above mixture was added NaHCO₃ (764 mg, 9.1 mmol) and FmocCl (565 mg, 9.1 mmol) in portions. The mixture was stirred at room temperature overnight, then washed with H₂O (3×300 mL) and the combined aqueous layers were extracted with EtOAc (3×30 mL). The combined organic layers were concentrated under reduced pressure and the residue was purified by silica gel column chromatography to give (2S,3S)-2-((((9H-fluoren-9-yl) methoxy) carbonyl) amino)-3-(4-bromothiazol-2-yl)-3-(dimethylamino) propanoic acid (320 mg, 60% yield) as a solid. LCMS (ESI): m/z [M+H]⁺ calc'd for C₂₃H₂₂BrN₃O₄S 515.1; found 516.0.

### Step 9.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl(S)-hexahydropyridazine-3-carboxylate (450 mg, 0.74 mmol) and DIPEA (1.60 g, 12.4 mmol) in DMF (30 mL) was added (2S)-2-((((9H-fluoren-9-yl) methoxy) carbonyl) amino)-3-(4-bromothiazol-2-yl)-3-(dimethylamino) propanoic acid (320 mg, 0.62 mmol) and HATU (471 mg, 1.24 mmol) in portions. The mixture was stirred at room temperature for 1 h, then washed with H₂O (3×30 mL) and the combined aqueous layers were extracted with EtOAc (3×30 mL). The combined organic layers were concentrated under reduced pressure and the residue was purified by silica gel column chromatography to give 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(3S)-1-((2S)-2-((((9H-fluoren-9-yl) methoxy) carbonyl) amino)-3-(4-bromothiazol-2-yl)-3-(dimethylamino) propanoyl) hexahydropyridazine-3-carboxylate (450 mg, 66% yield) as a solid. LCMS (ESI): m/z [M+H]⁺ calc'd for C₅₇H₆₉BBrN₇O₈S 1101.4; found 1102.5.

### Step 10.

To a mixture of 3-(1-ethyl-2-(2-((S)-1-methoxyethyl) pyridin-3-yl)-5-(4,4,5,5-tetramethyl-1,3,2-dioxaborolan-2-yl)-1H-indol-3-yl)-2,2-dimethylpropyl-(3S)-1-((2S)-2-((((9H-fluoren-9-yl) methoxy) carbonyl) amino)-3-(4-bromothiazol-2-yl)-3-(dimethylamino) propanoyl) hexahydropyridazine-3-carboxylate (450 mg, 0.41 mmol) and K₃PO₄ (217 mg, 1.0 mmol) in toluene (9 mL), 1,4-dioxane (3 mL) and H₂O (3 mL) under an atmosphere of N₂ was added Pd(dtbpf)Cl₂ (53 mg, 0.08 mmol) in portions. The mixture was heated to 60° C. and stirred for 1 h, then washed with H₂O (3×20 mL) and the combined aqueous layers extracted with EtOAc (3×20 mL). The combined organic layers were concentrated under reduced pressure and the residue was purified by silica gel column chromatography to give (9H-fluoren-9-yl) methyl ((6³S,4S,Z)-3-(dimethylamino)-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (70 mg, 19% yield) as an oil. LCMS (ESI): m/z [M+H]⁺ calc'd for C₅₁H₅₇N₇O₆S 895.4; found 896.3.

### Step 11.

A mixture of (9H-fluoren-9-yl) methyl ((6³S,4S,Z)-3-(dimethylamino)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (70 mg, 0.08 mmol) and piperidine (0.2 mL) in MECN (2 mL) was stirred at room temperature for 1 h. The mixture was concentrated under reduced pressure to give (6³S,4S,Z)-4-amino-3-(dimethylamino)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (60 mg, 71% yield) as an oil, which was used directly in the next step without further purification. LCMS (ESI): m/z [M+H]⁺ calc'd for C₃₆H₄₇NO₄S 673.3; found 674.1.

### Step 12.

To a mixture of (6³S,4S,Z)-4-amino-3-(dimethylamino)-1¹-ethyl-1²-(2-((S)-1-methoxyethyl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (60 mg, 0.09 mmol) and (1S,2S)-2-methylcyclopropane-1-carboxylic acid (9 mg, 0.09 mmol) in DMF (5 mL) was added DIPEA (230 mg, 1.78 mmol) and HATU (68 mg, 0.18 mmol) in portions. The mixture was stirred at room temperature for 1 h, then diluted with H₂O and extracted with EtOAc (2×20 mL). The combined organic layers were washed with H₂O (3×20 mL), dried over anhydrous Na₂SO₄ and filtered. The filtrate was concentrated under reduced pressure and the residue was purified by preparative-HPLC to give (1S,2S)—N-((6³S,3S,4S,Z)-3-(dimethylamino)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane- 4-yl)-2-methylcyclopropane-1-carboxamide (3.6 mg, 5% yield) as a solid. LCMS (ESI): m/z [M+H]⁺ calc'd for C₄₁H₅₃N₇O₅S 755.4; found 756.4; ¹H NMR (400 MHZ, CD₃OD) δ 8.66-8.62 (m, 1H), 8.44-8.39 (m, 1H), 7.86-7.79 (m, 1H), 7.63-7.54 (m, 2H), 7.48-7.43 (m, 1H), 7.41-7.37 (m, 1H), 6.01-5.94 (m, 1H), 4.26-3.95 (m, 6H), 3.94-3.81 (m, 2H), 3.47-3.38 (m, 1H), 3.07 (s, 3H), 2.80-2.72 (m, 1H), 2.67-2.58 (m, 1H), 2.55-2.45 (m, 1H), 2.38-2.13 (s, 6H), 1.91 (s, 2H), 1.63-1.40 (m, 3H), 1.39-1.32 (m, 3H), 1.29-1.08 (m, 7H), 1.06-0.91 (m, 8H), 0.84-0.75 (m, 1H), 0.69-0.41 (m, 7H).

Example A502. Synthesis of (1r,2R,3S)—N-((6³S,3S,4S,Z)-3-ethoxy-1¹-ethyl-1²-(2-((S)-1-10 methoxyethyl)-5-((1R,5S,7r)-9-(oxetan-3-yl)-3-oxa-9-azabicyclo[3.3.1]nonan-7-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

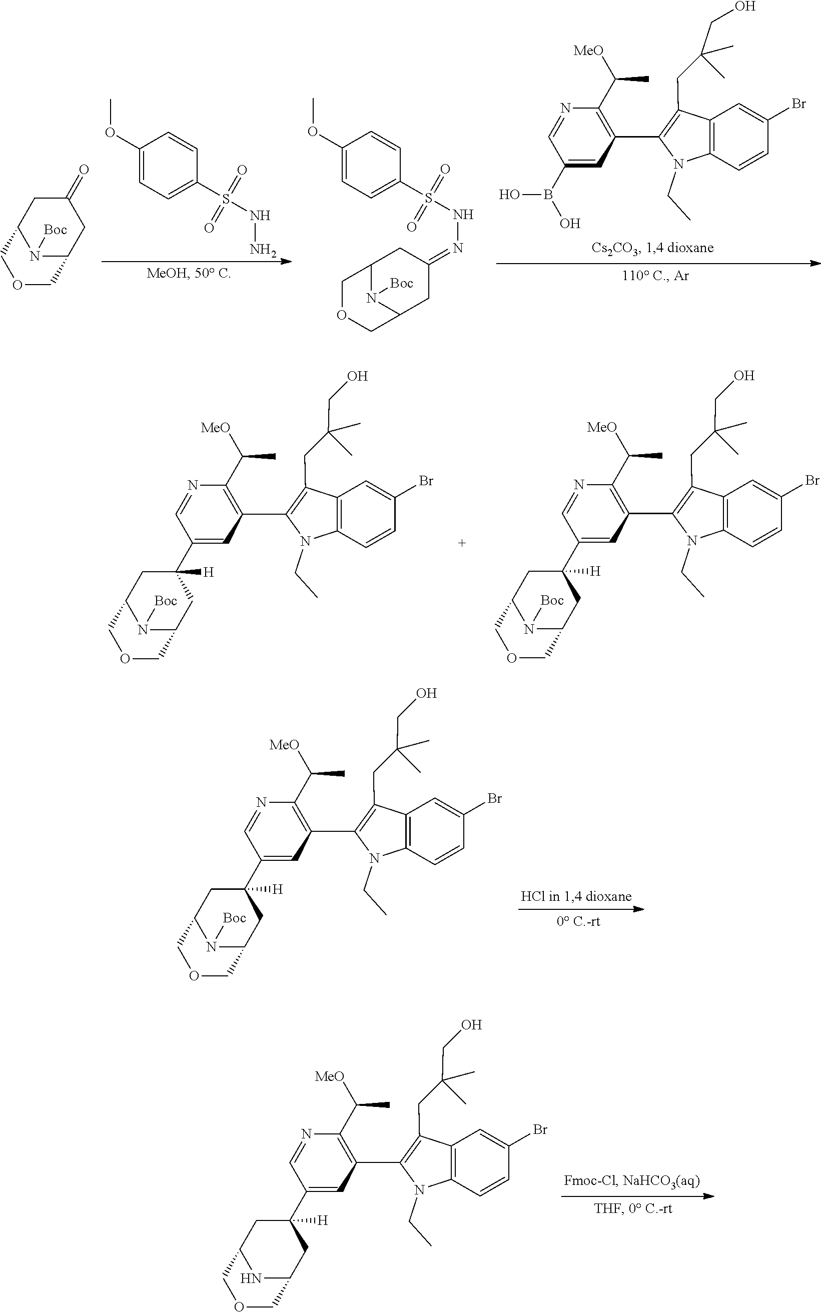

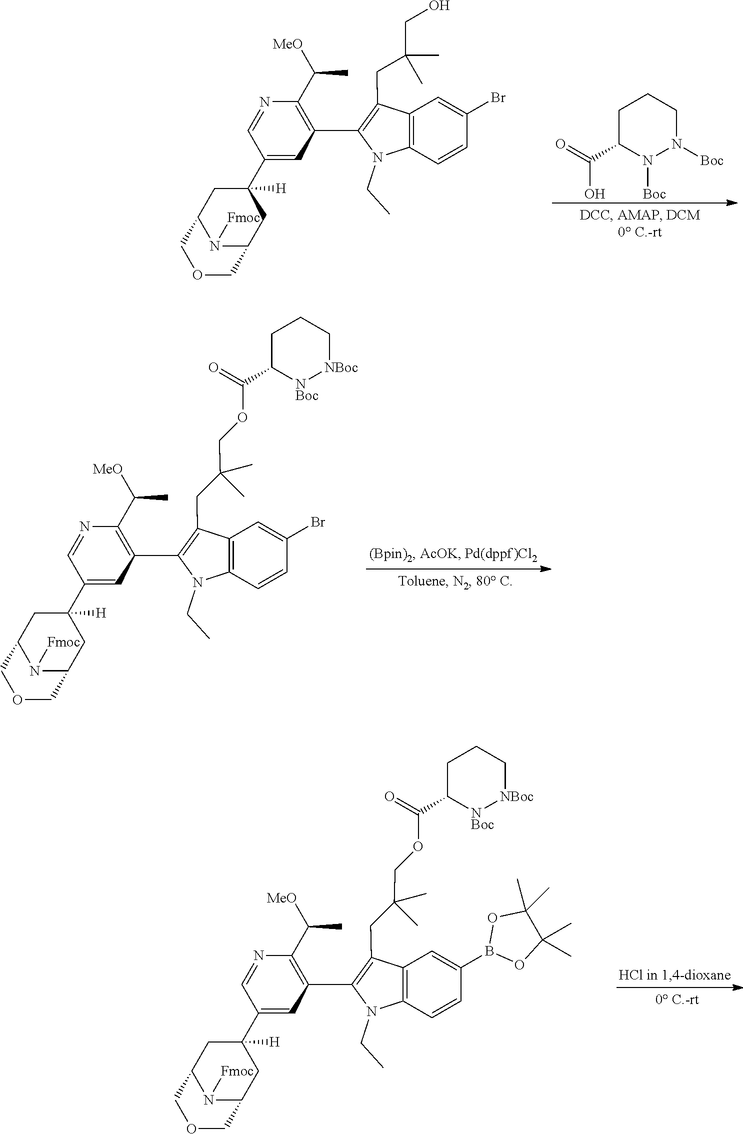

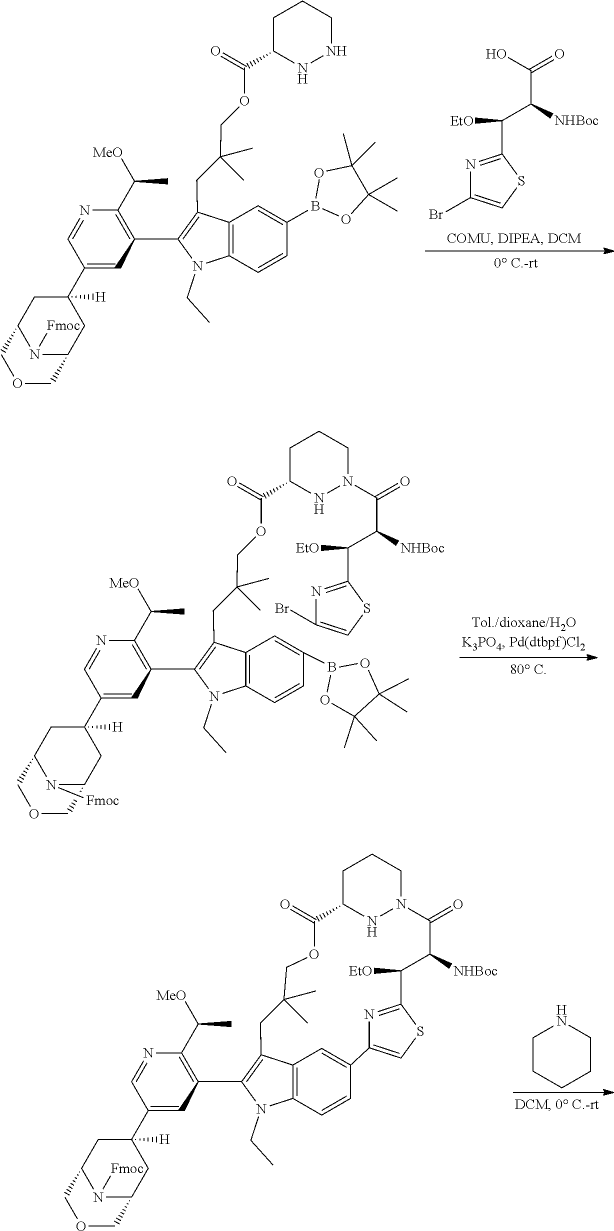

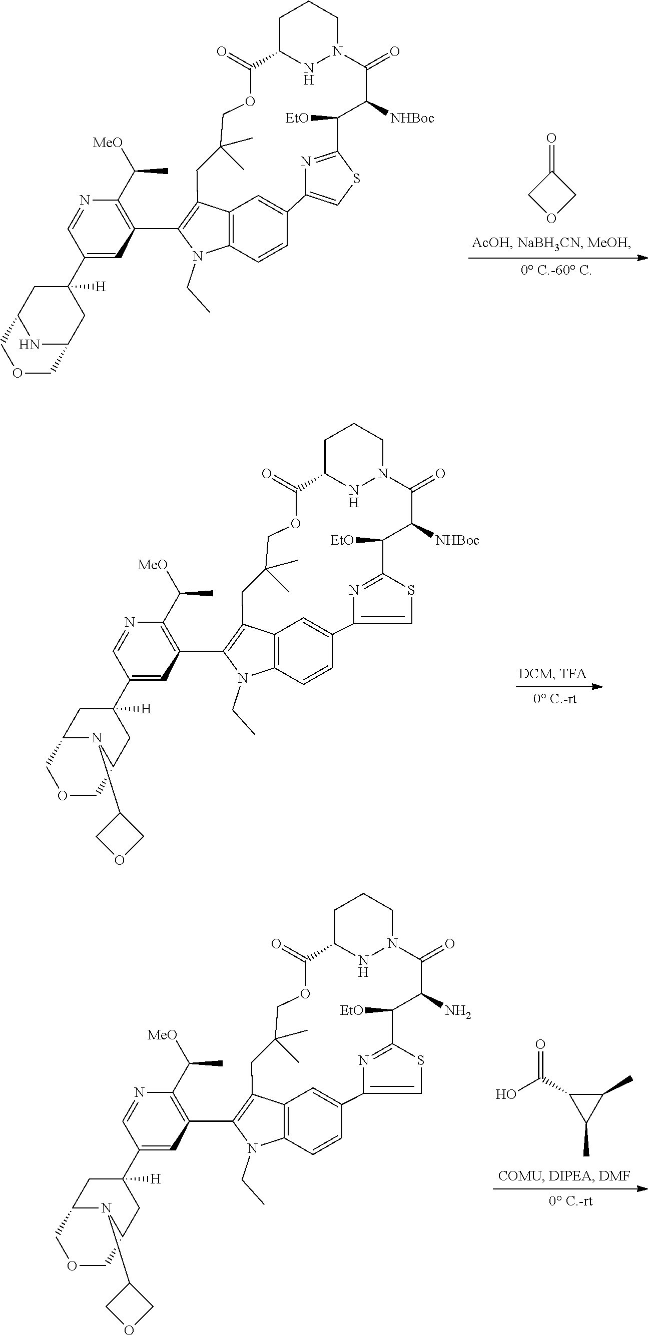

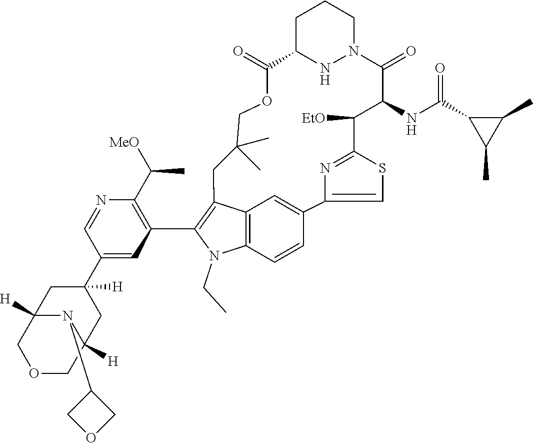

### Step 1.

To a mixture of tert-butyl (1R,5S)-7-oxo-3-oxa-9-azabicyclo[3.3.1]nonane-9-carboxylate (9.00 g, 37.3 mmol) in MeOH (90 mL) was added 4-methoxybenzenesulfonohydrazide (9.05 g, 44.8 mmol). The mixture was heated to 50° C. and stirred for 16 h, then concentrated under reduced pressure to give tert-butyl 7-(2-((4-methoxyphenyl) sulfonyl) hydrazineylidene)-3-oxa-9-azabicyclo[3.3.1]nonane-9-carboxylate (15.0 g) as a solid, which was used directly in the next step without further purification. LCMS (ESI): m/z [M+H]⁺ calc'd for C₁₉H₂₇N₃O₆S 425.2; found 426.3.

### Step 2.

A mixture of tert-butyl 7-(2-((4-methoxyphenyl) sulfonyl) hydrazineylidene)-3-oxa-9-azabicyclo[3.3.1]nonane-9-carboxylate (12.2 g, 28.7 mmol), (S)-(5-(5-bromo-1-ethyl-3-(3-hydroxy-2,2-dimethylpropyl)-1H-indol-2-yl)-6-(1-methoxyethyl) pyridin-3-yl) boronic acid (21.0 g, 43.0 mmol) and Cs₂CO₃ (14.0 g, 43.0 mmol) in 1,4-dioxane (120 mL) under an atmosphere of Ar was heated to 110° C. and stirred for 16 h. The mixture was concentrated under reduced pressure and the residue was purified by silica gel column chromatography and chiral-HPLC to give tert-butyl (1R,5S,7s)-7-(5-(5-bromo-1-ethyl-3-(3-hydroxy-2,2-dimethylpropyl)-1H-indol-2-yl)-6-((S)-1-methoxyethyl) pyridin-3-yl)-3-oxa-9-azabicyclo[3.3.1]nonane-9-carboxylate (3.3 g, 17% yield; RT=1.98 min) as solid and tert-butyl (1R,5S,7r)-7-(5-(5-bromo-1-ethyl-3-(3-hydroxy-2,2-dimethylpropyl)-1H-indol-2-yl)-6-((S)-1-methoxyethyl) pyridin-3-yl)-3-oxa-9-azabicyclo[3.3.1]nonane-9-carboxylate (4.5 g, 23% yield, RT=2.16 min) as a solid.

### Step 3.

To tert-butyl (1R,5S)-7-(5-(5-bromo-1-ethyl-3-(3-hydroxy-2,2-dimethylpropyl)-1H-indol-2-yl)-6-((S)-1-methoxyethyl) pyridin-3-yl)-3-oxa-9-azabicyclo[3.3.1]nonane-9-carboxylate (4.49 g, 6.7 mmol) was added HCl in 1,4-dioxane (40 mL, 10.0 mmol) at 0° C. The mixture was warmed to room temperature and stirred for 1 h, then concentrated under reduced pressure to give 3-(2-(5-((1R,5S,7r)-3-oxa-9-azabicyclo[3.3.1]nonan-7-yl)-2-((S)-1-methoxyethyl) pyridin-3-yl)-5-bromo-1-ethyl-1H-indol-3-yl)-2,2-dimethylpropan-1-ol (4.5 g, 92% yield) as a solid, which was used directly in the next step without further purification. LCMS (ESI): m/z [M+H]⁺ calc'd for C₃₀H₄₀BrN₃O₃ 569.2; found 570.3.

### Step 4.

To a mixture of 3-(2-(5-((1R,5S,7r)-3-oxa-9-azabicyclo[3.3.1]nonan-7-yl)-2-((S)-1-methoxyethyl) pyridin-3-yl)-5-bromo-1-ethyl-1H-indol-3-yl)-2,2-dimethylpropan-1-ol (4.49 g, 7.87 mmol) in THF (20 mL) at 0° C. was added 9H-fluoren-9-ylmethyl chloroformate (3.05 g, 11.8 mmol) in aqueous NaHCO₃ (20 mL) dropwise. The mixture was warmed to room temperature and stirred for 2 h, then extracted with EtOAc (3×20 mL). The combined organic layers were washed with H₂O (2×20 mL), dried over anhydrous Na₂SO₄ and filtered. The filtrate was concentrated under reduced pressure and the residue was purified by silica gel column chromatography to give (9H-fluoren-9-yl) methyl (1R,5S,7r)-7-(5-(5-bromo-1-ethyl-3-(3-hydroxy-2,2-dimethylpropyl)-1H-indol-2-yl)-6-((S)-1-methoxyethyl) pyridin-3-yl)-3-oxa-9-azabicyclo[3.3.1]nonane-9-carboxylate (5.8 g, 92% yield) as a solid. LCMS (ESI): m/z [M+H]⁺ calc'd for C₄₅H₅₀BrN₃O₅ 793.3; found 794.3 [for 81Br].

### Step 5.

To a mixture of (9H-fluoren-9-yl) methyl (1R,5S,7r)-7-(5-(5-bromo-1-ethyl-3-(3-hydroxy-2,2-dimethylpropyl)-1H-indol-2-yl)-6-((S)-1-methoxyethyl) pyridin-3-yl)-3-oxa-9-azabicyclo[3.3.1]nonane-9-carboxylate (5.8 g, 7.3 mmol) and(S)-1,2-bis (tert-butoxycarbonyl) hexahydropyridazine-3-carboxylic acid (4.83 g, 14.6 mmol) in DCM (50 mL) at 0° C. was added DMAP (0.38 g, 3.1 mmol) and DCC (2.55 g, 12.4 mmol) in portions. The mixture was warmed to room temperature and stirred for 2 h, then filtered and the filter cake was washed with DCM (3×30 mL). The filtrate was concentrated under reduced pressure and the residue was purified by silica gel column chromatography to give 3-(3-(2-(5-((1R,5S,7r)-9-(((9H-fluoren-9-yl) methoxy) carbonyl)-3-oxa-9-azabicyclo[3.3.1]nonan-7-yl)-2-((S)-1-methoxyethyl) pyridin-3-yl)-5-bromo-1-ethyl-1H-indol-3-yl)-2,2-dimethylpropyl) 1,2-di-tert-butyl-(S)-tetrahydropyridazine-1,2,3-tricarboxylate (7.6 g) as a solid. LCMS (ESI): m/z [M+H]⁺ calc'd for C₆₀H₇₄BrN₅O₁₀ 1105.5; found 1106.4 [for 81Br].

### Step 6.

To a mixture of 3-(3-(2-(5-((1R,5S,7r)-9-(((9H-fluoren-9-yl) methoxy) carbonyl)-3-oxa-9-azabicyclo[3.3.1]nonan-7-yl)-2-((S)-1-methoxyethyl) pyridin-3-yl)-5-bromo-1-ethyl-1H-indol-3-yl)-2,2-dimethylpropyl) 1,2-di-tert-butyl-(S)-tetrahydropyridazine-1,2,3-tricarboxylate (7.4 g, 6.7 mmol) and bis (pinacolato)diboron (8.50 g, 33.5 mmol) in toluene (70 mL) under an atmosphere of N₂ was added AcOK (2.63 g, 26.8 mmol) and Pd(dppf)Cl₂CH₂Cl₂ (1.09 g, 1.34 mmol). The mixture was heated to 80° C. and stirred for 3 h, then concentrated under reduced pressure. The residue was purified by silica gel column to chromatography give 3-(3-(2-(5-((1R,5S,7r)-9-(((9H-fluoren-9-yl) methoxy) carbonyl)-3-oxa-9-azabicyclo[3.3.1]nonan-7-yl)-2-((S)-1-methoxyethyl) pyridin-3-yl)-1-ethyl-5-(4,4,5,5-tetramethyl-1,3,2-dioxaborolan-2-yl)-1H-indol-3-yl)-2,2-dimethylpropyl) 1,2-di-tert-butyl-(S)-tetrahydropyridazine-1,2,3-tricarboxylate (6.9 g, 89% yield) as a solid. LCMS (ESI): m/z [M+H]⁺ calc'd for C₆₆H₈₆BN₅O₁₂ 1151.6; found 1152.6.

### Step 7.

To 3-(3-(2-(5-((1R,5S,7r)-9-(((9H-fluoren-9-yl) methoxy) carbonyl)-3-oxa-9-azabicyclo[3.3.1]nonan-7-yl)-2-((S)-1-methoxyethyl) pyridin-3-yl)-1-ethyl-5-(4,4,5,5-tetramethyl-1,3,2-dioxaborolan-2-yl)-1H-indol-3-yl)-2,2-dimethylpropyl) 1,2-di-tert-butyl-(S)-tetrahydropyridazine-1,2,3-tricarboxylate (6.9 g, 6.0 mmol) was added HCl in 1,4-dioxane (60 mL) at 0° C. The mixture was warmed to room temperature and was stirred for 4 h, then concentrated under reduced pressure to give (9H-fluoren-9-yl) methyl (1R,5S,7r)-7-(5-(1-ethyl-3-(3-(((S)-hexahydropyridazine-3-carbonyl)oxy)-2,2-dimethylpropyl)-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1]nonane-9-carboxylate (6.47 g) as a solid. LCMS (ESI): m/z [M+H]⁺ calc'd for C₅₆H₇₀BN₅O₈ 951.5; found 952.6.

### Step 8.

To a mixture of (9H-fluoren-9-yl) methyl (1R,5S,7r)-7-(5-(1-ethyl-3-(3-(((S)-hexahydropyridazine-3-carbonyl)oxy)-2,2-dimethylpropyl)-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1]nonane-9-carboxylate (6.45 g, 6.78 mmol) and (2S,3S)-3-(4-bromothiazol-2-yl)-2-((tert-butoxycarbonyl) amino)-3-ethoxypropanoic acid (3.21 g, 8.13 mmol) in DCM (60 mL) at 0° C. was added DIPEA (8.76 g, 67.8 mmol) and COMU (3.48 g, 8.1 mmol) in portions. The mixture was warmed to room temperature and stirred for 2 h, then concentrated under reduced pressure and the residue was purified by silica gel column chromatography to give (9H-fluoren-9-yl) methyl (1R,5S,7r)-7-(5-(3-(3-(((S)-1-((2S,3S)-3-(4-bromothiazol-2-yl)-2-((tert-butoxycarbonyl) amino)-3-ethoxypropanoyl) hexahydropyridazine-3-carbonyl)oxy)-2,2-dimethylpropyl)-1-ethyl-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1]nonane-9-carboxylate (6.9 g, 76% yield) as a solid. LCMS (ESI): m/z [M+H]⁺ calc'd for C₆₉H₈₇BBrN₇O₁₂S 1329.5; found 1330.5.

### Step 9.

To a mixture of (9H-fluoren-9-yl) methyl (1R,5S,7r)-7-(5-(3-(3-(((S)-1-((2S,3S)-3-(4-bromothiazol-2-yl)-2-((tert-butoxycarbonyl) amino)-3-ethoxypropanoyl) hexahydropyridazine-3-carbonyl)oxy)-2,2-dimethylpropyl)-1-ethyl-5-(4,4,5,5-tetramethyl-1,3,2-dioxaborolan-2-yl)-1H-indol-2-yl)-6-((S)-1-methoxyethyl) pyridin-3-yl)-3-oxa-9-azabicyclo[3.3.1]nonane-9-carboxylate (6.9 g, 5.2 mmol) in toluene (300 mL), 1,4-dioxane (100 mL) and H₂O (100 mL) under an atmosphere of N₂ was added K₃PO₄ (3.31 g, 15.6 mmol) and Pd(DtBPF)Cl₂ (0.42 g, 0.52 mmol). The mixture was heated to 80° C. and stirred for 3 h, then concentrated under reduced pressure and the residue was purified by silica gel column chromatography to give (9H-fluoren-9-yl) methyl (1R,5S,7r)-7-(5-((6³S,3S,4S,Z)-4-((tert-butoxycarbonyl) amino)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-12-yl)-6-((S)-1-methoxyethyl) pyridin-3-yl)-3-oxa-9-azabicyclo[3.3.1]nonane-9-carboxylate (3.8 g, 65% yield) as a solid. LCMS (ESI): m/z [M+H]⁺ calc'd for C₆₃H₇₅N₇O₁₀S 1121.5; found 1123.3.

### Step 10.

To a mixture of (9H-fluoren-9-yl) methyl (1R,5S,7r)-7-(5-((6³S,3S,4S,Z)-4-((tert-butoxycarbonyl) amino)-3-ethoxy-1¹-ethyl-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-12-yl)-6-((S)-1-methoxyethyl) pyridin-3-yl)-3-oxa-9-azabicyclo[3.3.1]nonane-9-carboxylate (3.8 g, 3.4 mmol) in DCM (40 mL) at 0° C. was added piperidine (1.44 g, 16.9 mmol) dropwise. The mixture was warmed to room temperature and stirred for 5 h, then concentrated under reduced pressure to give tert-butyl ((6³S,3S,4S,Z)-12-(5-((1R,5S,7r)-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl) carbamate (4.7 g) as a solid, which was used directly in the next step without further purification. LCMS (ESI): m/z [M+H]⁺ calc'd for C₄₈H₆₅N₇O₈S 899.5; found 900.6.

### Step 11.

To a mixture of tert-butyl ((6³S,3S,4S,Z)-12-(5-((1R,5S,7r)-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (1.12 g, 15.6 mmol) in MeOH (2 mL) at 0° C. was added AcOH (467 mg, 7.8 mmol). The mixture was stirred for 30 min, then NaBH₃CN (147 mg, 2.33 mmol) was added, and the mixture was heated to 60° C. and stirred for 3 h. The mixture was concentrated under reduced pressure and the residue was purified by silica gel column chromatography to give tert-butyl ((6³S,3S,4S,Z)-3-ethoxy-11-ethyl-1²-(2-((S)-1-methoxyethyl)-5-((1R,5S,7r)-9-(oxetan-3-yl)-3-oxa-9-azabicyclo[3.3.1]nonan-7-yl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (251 mg, 33% yield) as a solid. LCMS (ESI): m/z [M+H]⁺ calc'd for C₅₁H₆₉N₇O₉S 955.5; found 956.5.

### Step 12.

To a mixture of tert-butyl ((6³S,3S,4S,Z)-3-ethoxy-1¹-ethyl-1²-(2-((S)-1-methoxyethyl)-5-((1R,5S,7r)-9-(oxetan-3-yl)-3-oxa-9-azabicyclo[3.3.1]nonan-7-yl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (250 mg, 0.26 mmol) in DCM (2 mL) at 0° C. was added TFA (0.4 mL). The mixture was warmed to room temperature and stirred for 30 min, then concentrated under reduced pressure to give (6³S,3S,4S,Z)-4-amino-3-ethoxy-1¹-ethyl-1²-(2-((S)-1-methoxyethyl)-5-((1R,5S,7r)-9-(oxetan-3-yl)-3-oxa-9-azabicyclo[3.3.1]nonan-7-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (310 mg) as a solid, that was used directly in the next step without further purification. LCMS (ESI): m/z [M+H]⁺ calc'd for C₄₆H₆₁N₇O₇S 855.4; found 856.5.

### Step 13.

To a mixture of (6³S,3S,4S,Z)-4-amino-3-ethoxy-1¹-ethyl-1²-(2-((S)-1-methoxyethyl)-5-((1R,5S,7r)-9-(oxetan-3-yl)-3-oxa-9-azabicyclo[3.3.1]nonan-7-yl) pyridin-3-yl)-10, 10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (310 mg, 0.36 mmol) and (1r,2R,3S)-2,3-dimethylcyclopropane-1-carboxylic acid (62 mg, 0.54 mmol) in DMF (3 mL) at 0° C. was added DIPEA (468 mg, 3.62 mmol) and COMU (155 mg, 0.36 mmol) in portions. The mixture was warmed to room temperature and stirred for 1 h, then purified by preparative-HPLC to give (1r,2R,3S)—N-((6³S,3S,4S,Z)-3-ethoxy-1¹-ethyl-1²-(2-((S)-1-methoxyethyl)-5-((1R,5S,7r)-9-(oxetan-3-yl)-3-oxa-9-azabicyclo[3.3.1]nonan-7-yl) pyridin-3-yl)-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2,3-dimethylcyclopropane-1-carboxamide (113 mg, 32% yield) as a solid. LCMS (ESI): m/z [M+H]⁺ calc'd for C₅₂H₆₉N₇O₈S 951.5; found 952.5; ¹H NMR (300 MHZ, DMSO-d₆) δ 8.66 (d, J=2.1 Hz, 1H), 8.51 (s, 1H), 7.92 (s, 1H), 7.76 (d, J=8.4 Hz, 2H), 7.61 (dd, J=15.3, 9.2 Hz, 2H), 5.88 (d, J=9.9 Hz, 1H), 5.18 (d, J=11.6 Hz, 1H), 4.93 (s, 1H), 4.59-4.41 (m, 3H), 4.32 (s, 2H), 4.24 (d, J=6.2 Hz, 2H), 4.13 (s, 3H), 3.82 (d, J=11.0 Hz, 4H), 3.69-3.49 (m, 4H), 3.19 (s, 3H), 2.84-2.74 (s, 2H), 2.64 (s, 2H), 2.10-1.94 (m, 3H), 1.76 (s, 4H), 1.53 (s, 1H), 1.37 (d, J=6.1 Hz, 3H), 1.24 (s, 1H), 1.19 (d, J=7.0 Hz, 3H), 1.15 (s, 2H), 1.07 (d, J=7.2 Hz, 6H), 0.93 (t, J=7.0 Hz, 3H), 0.84 (s, 3H), 0.41 (s, 3H).

### Example A503. Synthesis of (1r,2R,3S)—N-((6³S,3S,4S,Z)-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2,3-dimethylcyclopropane-1-carboxamide

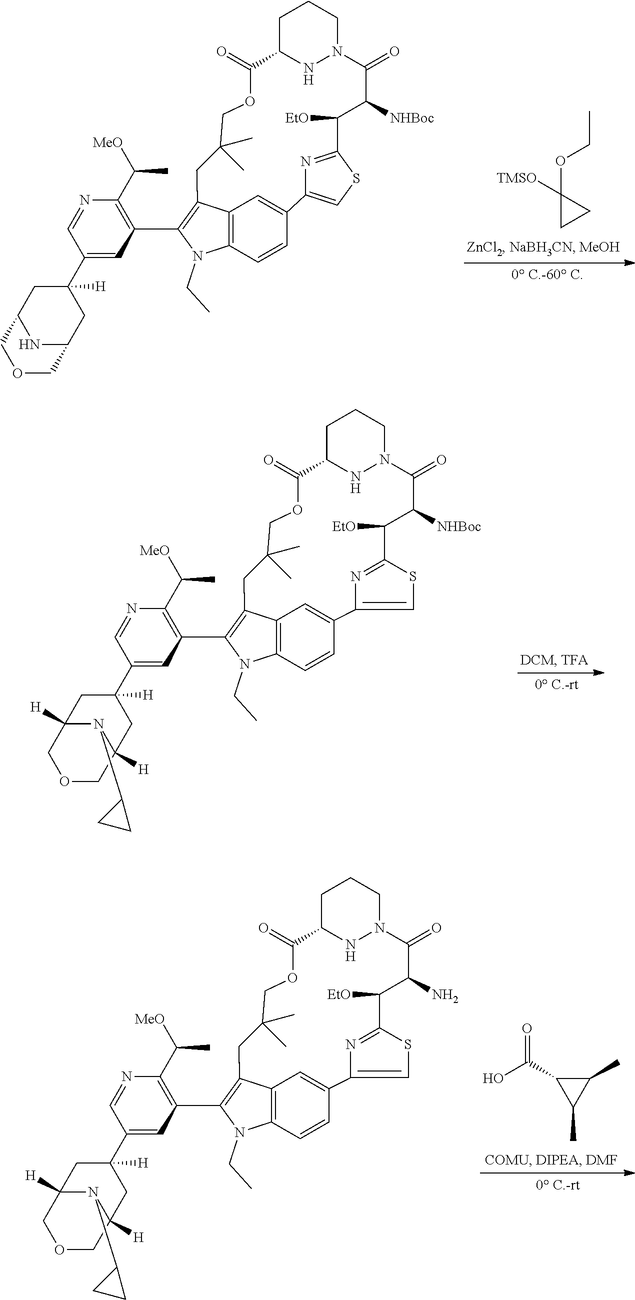

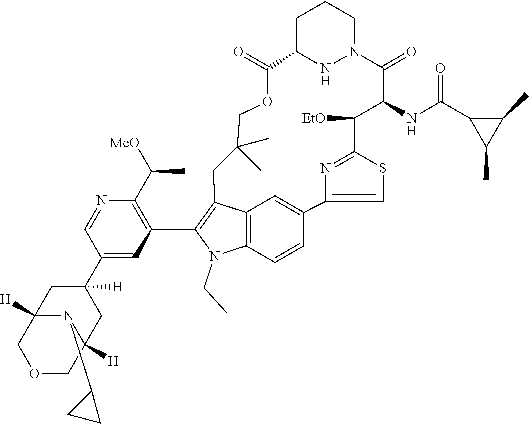

### Step 1.

To a mixture of tert-butyl ((6³S,3S,4S,Z)-12-(5-((1R,5S,7r)-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (300 mg, 0.33 mmol) and (1-ethoxycyclopropoxy) trimethylsilane (1.16 g, 6.6 mmol) in MeOH (1.5 mL) at 0° C. was added AcOH (200 mg, 3.3 mmol) dropwise. The mixture was stirred at 0° C. for 30 min, then NaBH₃CN (105 mg, 1.67 mmol) was added, the mixture was heated to 60° C. and stirred for 2 h. The residue was purified by silica gel column chromatography to give tert-butyl ((6³S,3S,4S,Z)-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-11-ethyl-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (235 mg, 75% yield) as a solid. LCMS (ESI): m/z [M+H]⁺ calc'd for C₅₁H₆₉N₇O₈S 939.5; found 940.3.

### Step 2.

To a mixture of tert-butyl ((6³S,3S,4S,Z)-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl) carbamate (230 mg, 0.25 mmol) in DCM (2 mL) at 0° C. was added TFA (0.4 mL) dropwise. The mixture was warmed to room temperature and stirred for 1 h, then concentrated under reduced pressure to give (6³S,3S,4S,Z)-4-amino-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-11-ethyl-10,10-dimethyl-6¹,6²,6³,6⁴,6⁵,6⁶-hexahydro-1¹H-8-oxa-2 (4,2)-thiazola-1 (5,3)-indola-6 (1,3)- pyridazinacycloundecaphane-5,7-dione (340 mg) as a solid. LCMS (ESI): m/z [M+H]⁺ calc'd for C₄₆H₆₁N₇O₆S 839.4; found 840.5.

### Step 3.

To a mixture of (6³S,3S,4S,Z)-4-amino-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-1¹-ethyl-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (200 mg, 0.24 mmol) and (1r,2R,3S)-2,3-dimethylcyclopropane-1-carboxylic acid (41 mg, 0.36 mmol) in DMF (2 mL) at 0° C. was added DIPEA (308 mg, 2.4 mmol) and COMU (102 mg, 0.24 mmol) in portions. The mixture was warmed to room temperature and stirred for 1 h, then purified by preparative-HPLC to give (1r,2R,3S)—N-((6³S,3S,4S,Z)-12-(5-((1R,5S,7r)-9-cyclopropyl-3-oxa-9-azabicyclo[3.3.1]nonan-7-yl)-2-((S)-1-methoxyethyl) pyridin-3-yl)-3-ethoxy-1¹-ethyl-10, 10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2,3-dimethylcyclopropane-1-carboxamide (78 mg, 35% yield) as a solid. LCMS (ESI): m/z [M+H]⁺ calc'd for C₅₂H₆₉N₇O₇S 935.5; found 936.4; ¹H NMR (300 MHZ, DMSO-d₆) δ 8.73 (m, 1H), 8.50 (m, 1H), 7.93 (s, 1H), 7.82-7.53 (m, 3H), 5.88 (d, J=9.8 Hz, 1H), 5.22 (d, J=9.7 Hz, 1H), 4.92 (s, 2H), 4.55-3.86 (m, 14H), 3.84-3.37 (m, 7H), 3.21 (s, 3H), 2.81 (d, J=12.3 Hz, 2H), 2.32 (s, 3H), 2.02 (d, 1H), 1.82 (s, 2H), 1.53 (t, J=3.9 Hz, 2H), 1.38 (d, J=6.0 Hz, 3H), 1.12 (dt, J=34.8, 6.3 Hz, 13H), 0.86 (s, 7H), 0.55 (s, 3H).

### Example A504. Synthesis of presumed (1S,2R,3S)—N-((6³S,3S,4S,Z)-3-ethoxy-1¹-ethyl-12-(2-((S)-1-methoxyethyl) pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2-methyl-3-(pyrimidin-4-yl) cyclopropane-1-carboxamide

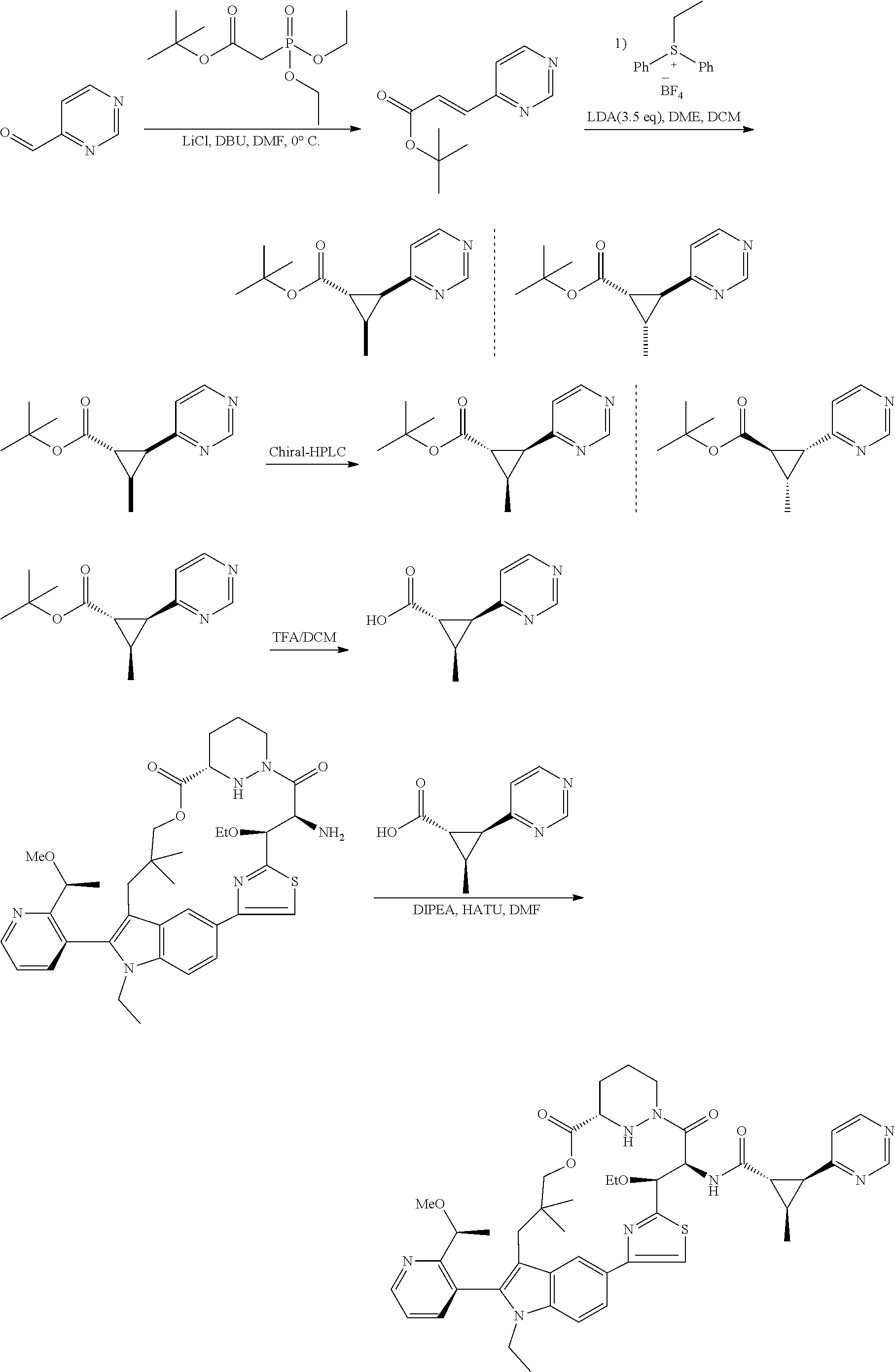

### Step 1.

To a mixture of LiCl (0.31 g, 7.4 mmol) and pyrimidine-4-carbaldehyde (1.0 g, 9.3 mmol) in DMF at 0° C. was added DBU (1.69 g, 11.1 mmol) and tert-butyl 2-(diethoxyphosphoryl) acetate (2.80 g, 11.1 mmol) dropwise. The mixture was warmed to room temperature and stirred for 1 h at room temperature, then cooled to 0° C., quenched with saturated NH₄Cl and extracted with EtOAc (200 mL). The organic layer was washed with brine (3×200 mL) and concentrated under reduced pressure. The residue was purified by silica gel column chromatography to give tert-butyl (E)-3-(pyrimidin-4-yl) acrylate (1.0 g, 52% yield) as a solid. LCMS (ESI): m/z [M+H]⁺ calc'd for C₁₁H₁₄N₂O₂ 206.1; found 207.1.

### Step 2.

To a mixture ethyldiphenylsulfanium tetrafluoroborate (2.2 g, 10.2 mmol) in DME and DCM (10:1) at −60° C. under an atmosphere of N₂ was treated with LDA, 2M in THF (6.0 mL, 12.0 mmol) for 0.5 h. The mixture was warmed to room temperature and tert-butyl (E)-3-(pyrimidin-4-yl) acrylate (700 mg, 3.4 mmol) was added dropwise. The mixture was stirred at room temperature 1 h, then quenched with saturated NH₄Cl and extracted with EtOAc (3×100 mL). The combined organic layers were concentrated under reduced pressure and the residue was purified by preparative-HPLC to give 2-methyl-3-(pyrimidin-4-yl) cyclopropane-1-carboxylic acid (160 mg, 20% yield) as a solid. LCMS (ESI): m/z [M+H]⁺ calc'd for C₁₃H₁₈N₂O₂ 234.1; found 235.1; ¹H NMR (300 MHZ, CDCl₃) δ 9.10 (s, 1H), 8.63 (s, 1H), 7.33 (s, 1H), 2.66 (dd, J=9.7, 4.7 Hz, 1H), 2.46 (dd, J=5.7, 4.7 Hz, 1H), 2.06-1.78 (m, 1H), 1.47 (s, 9H), 1.13 (d, J=6.4 Hz, 3H).

A racemic mixture of the above compound was separated by chiral-HPLC to give (55 mg, single diastereomer of unknown absolute configuration, RT=6.2 min) as a solid and (61 mg, single diastereomer of unknown absolute configuration, RT=7.3 min).

### Step 3.

A mixture of tert-butyl (1S,2R,3S)-2-methyl-3-(pyrimidin-4-yl) cyclopropane-1-carboxylate (50 mg, 0.21 mmol, single diastereomer of unknown absolute configuration; RT=6.2 min) and TFA (5 mL, 67.3 mmol) in DCM was stirred at room temperature for 1 h, then concentrated under reduced pressure to give (1S,2R,3S)-2-methyl-3-(pyrimidin-4-yl) cyclopropane-1-carboxylic acid, that was used directly in the next step without further purification (single diastereomer of unknown absolute configuration). LCMS (ESI): m/z [M+H]⁺ calc'd for C₉H₁₀N₂O₂ 178.0; found 179.1.

### Step 4.

To a mixture of (1S,2R,3S)-2-methyl-3-(pyrimidin-4-yl) cyclopropane-1-carboxylic acid (53 mg, 0.3 mmol) and DIPEA (192 mg, 1.48 mmol) in DMF at room temperature under an atmosphere of N₂ was added (6³S,3S,4S,Z)-4-amino-3-ethoxy-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (100 mg, 0.15 mmol) and HATU (113 mg, 0.3 mmol). The mixture was stirred at room temperature for 1 h, then diluted with EtOAc (100 mL) and washed with brine (3×100 mL). The organic layer was concentrated under reduced pressure and the residue was purified by preparative-HPLC to give (1S,2R,3S)—N-((6³S,3S,4S,Z)-3-ethoxy-1¹-ethyl-1²-(2-((S)-1-methoxyethyl) pyridin-3-yl)-10, 10-dimethyl-5,7-dioxo-61, 62,63,64,65,66-hexahydro-1¹H-8-oxa-2 (4,2)-thiazola-1 (5,3)-indola-6 (1,3)-pyridazinacycloundecaphane-4-yl)-2-methyl-3-(pyrimidin-4-yl) cyclopropane-1-carboxamide (19 mg, 15% yield; single diastereomer of unknown absolute configuration) as a solid. LCMS (ESI): m/z [M+H]⁺ calc'd for C₄₅H₅₄N₈O₆S 834.4; found 835.2; ¹H NMR (300 MHZ, DMSO-d₆) δ 9.09 (s, 1H), 8.82-8.71 (m, 1H), 8.66 (m, 1H), 8.51 (s, 1H), 8.23-8.22 (m, 1H), 7.94 (s, 1H), 7.87-7.65 (m, 2H), 7.63-7.30 (m, 3H), 5.89-5.76 (m, 1H), 5.19-5.15 (m, 1H), 4.95 (s, 1H), 4.28-4.13 (m, 5H), 3.79-3.52 (m, 7H), 3.42-3.31 (m, 3H), 2.83 (s, 2H), 2.08 (s, 1H), 1.78 (s, 3H), 1.38-1.34 (m, 3H), 1.31-1.20 (m, 6H), 0.88-0.78 (m, 6H), 0.41 (s, 3H).

### Example A505. Synthesis of presumed (1R,2S,3R)—N-((6³S,3S,4S,Z)-3-ethoxy-1¹-ethyl-12-(2-((S)-1-methoxyethyl) pyridin-3-yl)-10,10-dimethyl-5,7-dioxo-6¹,6²,6³,6⁴,6⁵,6⁶-hexahydro-11H-8-oxa-2 (4,2)-thiazola-1 (5,3)-indola-6 (1,3)-pyridazinacycloundecaphane-4-yl)-2-methyl-3-(pyrimidin-4-yl) cyclopropane-1-carboxamide

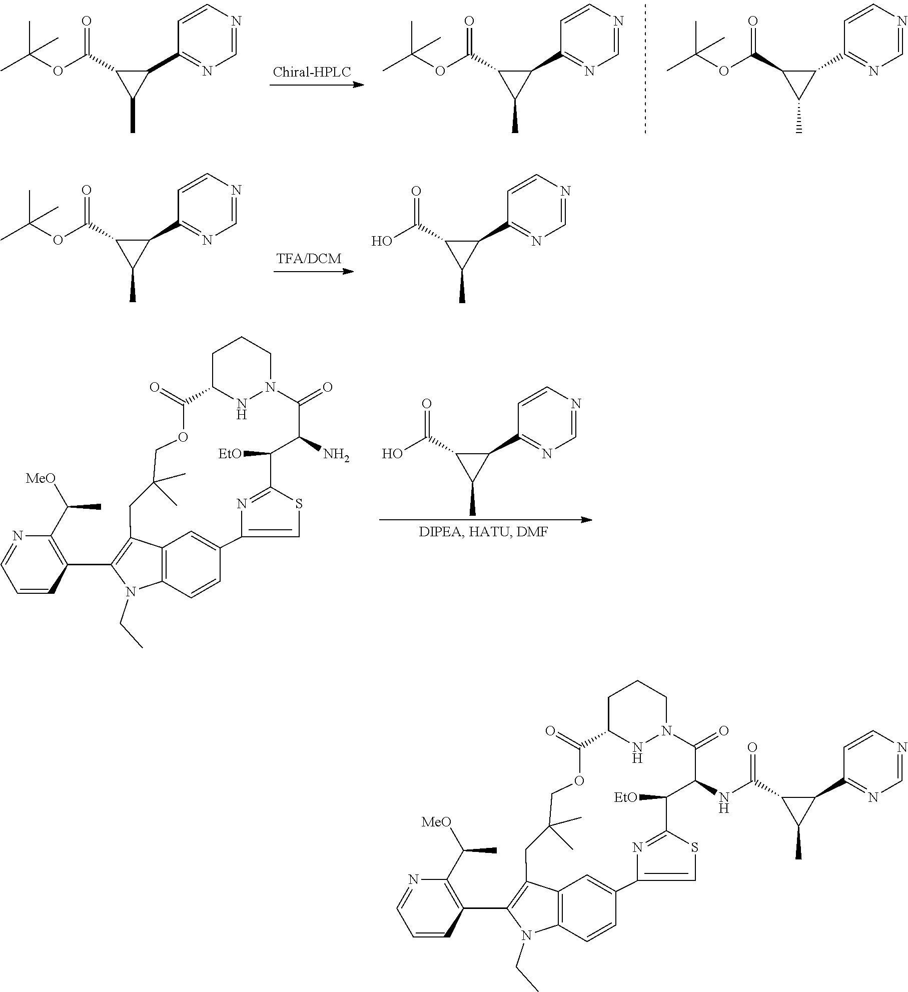

### Step 1.

A racemic mixture of the starting cyclopropane was separated by chiral HPLC [condition (Column: CHIRALPAK IF, 2×25 cm, 5 μM; Mobile Phase A: Hexane (10 mM NH₃-MeOH), Mobile Phase B: IPA-HPLC; Flow rate: 20 mL/min; Gradient: 5% B to 5% B in 10 min; Wave Length: 252/220 nm; RT1 (min): 6.2; RT2 (min): 7.3; Sample Solvent: MeOH:DCM=1:1; Injection Volume: 0.2 mL; Number Of Runs: 11)]. Product A (single diastereomer of unknown absolute configuration, 55 mg, RT=6.2 min) as white solid; Product B (single diastereomer of unknown absolute configuration, 61 mg, RT=7.3 min).

### Step 2.

A mixture of presumed tert-butyl (1R,2S,3R)-2-methyl-3-(pyrimidin-4-yl) cyclopropane-1-carboxylate (single diastereomer of unknown absolute configuration, 50 mg, 0.21 mmol) and TFA (5 mL, 67.3 mmol) in DCM was stirred at room temperature for 1 h, then concentrated under reduced pressure to give presumed (1R,2S,3R)-2-methyl-3-(pyrimidin-4-yl) cyclopropane-1-carboxylic acid (single diastereomer of unknown absolute configuration). LCMS (ESI): m/z [M+H]⁺ calc'd for C₉H₁₀N₂O₂ 178.0; found 179.1.

### Step 3.

To a mixture of (1R,2S,3R)-2-methyl-3-(pyrimidin-4-yl) cyclopropane-1-carboxylic acid (53 mg, 0.3 mmol) and DIPEA (192 mg, 1.48 mmol) in DMF at room temperature under an atmosphere of N₂ was added (6³S,3S,4S,Z)-4-amino-3-ethoxy-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (100 mg, 0.15 mmol) and HATU (113 mg, 0.3 mmol). The mixture was stirred at room temperature for 1 h, then diluted with EtOAc (100 mL) and washed with brine (3×100 mL). The organic layer was concentrated under reduced pressure and the residue was purified by preparative-HPLC to give (1R,2S,3R)—N-((6³S,3S,4S,Z)-3-ethoxy-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methyl-3-(pyrimidin-4-yl) cyclopropane-1-carboxamide (single diastereomer of unknown absolute configuration, 29 mg, 19% yield) as a solid. LCMS (ESI): m/z [M+H]⁺ calc'd for C₄₅H₅₄N₈O₆S 834.4; found 835.2; ¹H NMR (300 MHz, DMSO-d₆) δ 9.10 (s, 1H), 8.83-8.78 (m, 1H), 8.71 (m, 1H), 8.54 (s, 1H), 8.29-8.24 (m, 1H), 7.98 (s, 1H), 7.88-7.78 (m, 2H), 7.74-7.69 (m, 1H), 7.67-7.35 (m, 3H), 5.85-5.72 (m, 1H), 5.19-5.12 (m, 1H), 4.93 (s, 1H), 4.30-4.22 (m, 5H), 3.80-3.60 (m, 7H), 3.46-3.38 (m, 3H), 2.88 (s, 1H), 2.75-2.56 (m, 1H), 2.18 (s, 1H), 1.88 (s, 1H), 1.71-1.56 (m, 2H), 1.48-1.24 (m, 3H), 1.28-1.22 (m, 3H), 1.19-1.02 (m, 3H), 0.98-0.88 (m, 6H), 0.44 (s, 3H).

### Examples A506 and A507. Synthesis of presumed (1R,2S,3R)—N-((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methyl-3-(1-methyl-1H-imidazol-4-yl) cyclopropane-1-carboxamide and (1R,2S,3R)—N-((6³S,3S,4S,Z)-3-ethoxy-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methyl-3-(1-methyl-1H-imidazol-4-yl) cyclopropane-1-carboxamide

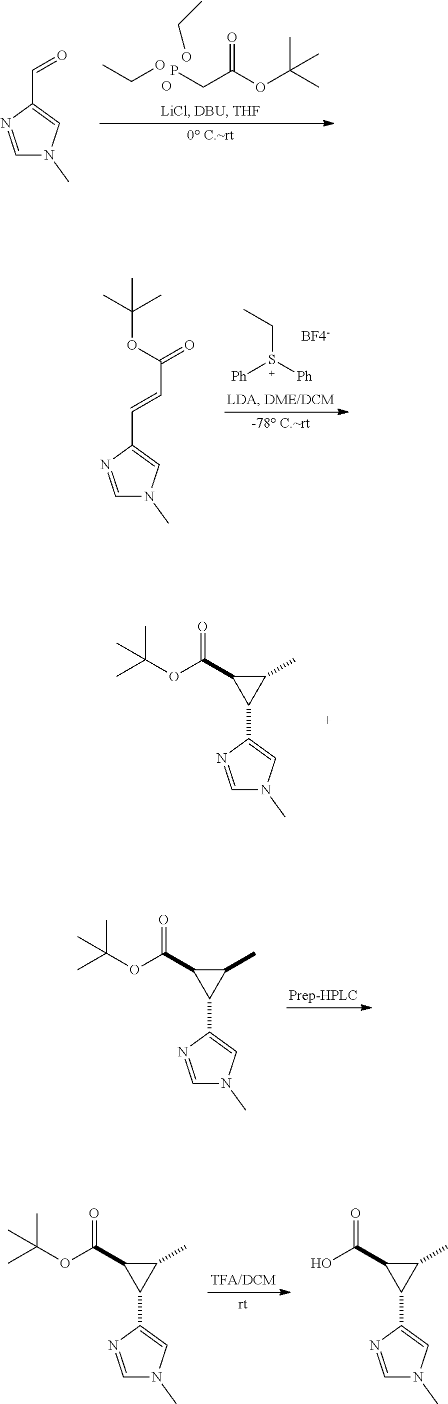

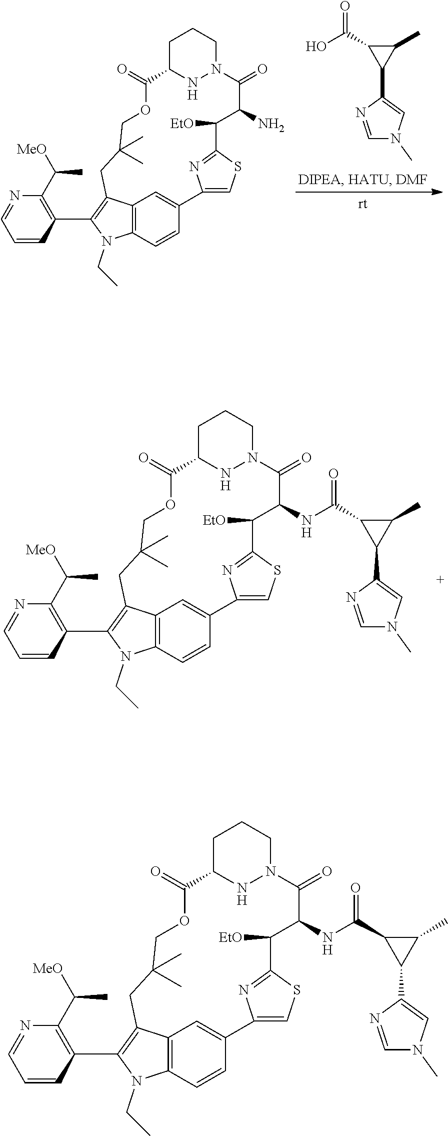

### Step 1.

To a mixture of 1-methyl-1H-imidazole-4-carbaldehyde (1.9 g, 17.3 mmol) and LiCl (0.95 g, 22.4 mmol) in THF (10 mL) at 0° C. were added tert-butyl 2-(diethoxyphosphoryl) acetate (5.66 g, 22.4 mmol) and DBU (2.63 g, 17.3 mmol) in portions. The mixture was allowed to warm to room temperature and stirred for 2 h then the mixture was washed H₂O (2×30 mL). The combined aqueous layers were extracted with EtOAc (3×30 mL) and the combined organic layers were dried and filtered. The filtrate was concentrated under reduced pressure and the residue was purified by silica gel column chromatography to give tert-butyl (E)-3-(1-methyl-1H-imidazol-4-yl) acrylate (3 g, 84% yield) as an oil. LCMS (ESI): m/z [M+H]⁺ calc'd for C₁₁H₁₆N₂O₂ 208.1; found 209.1.

### Step 2.

To a mixture of ethyldiphenylsulfonium tetrafluoroborate (2.18 g, 7.2 mmol) in DCM: DME (1:10) at −60° C. under an atmosphere of N₂ was treated with 2M LDA in THF (12 mL, 24 mmol) for 30 min, followed by the addition of tert-butyl (E)-3-(1-methyl-1H-imidazol-4-yl) acrylate (500 mg, 2.4 mmol) in portions. The mixture was warmed to room temperature and stirred for 2 h, then quenched with saturated NH₄Cl and extracted with EtOAc (3×50 mL). The combined organic layers were dried. The residue was purified by preparative-HPLC to give tert-butyl (1S,2R,3S)-2-methyl-3-(1-methyl-1H-imidazol-4-yl) cyclopropane-1-carboxylate (200 mg, 35% yield of a single diastereomer of unknown absolute configuration) and tert-butyl (1S,2S,3S)-2-methyl-3-(1-methyl-1H-imidazol-4-yl) cyclopropane-1-carboxylate (110 mg, 19% yield of a single diastereomer of unknown absolute configuration), as an oil. LCMS (ESI): m/z [M+H]⁺ calc'd for C₁₃H₂₀N₂O₂ 236.2; found 236.9.

### Step 3.

A mixture of presumed tert-butyl (1S,2R,3S)-2-methyl-3-(1-methyl-1H-imidazol-4-yl) cyclopropane-1-carboxylate (70 mg, 0.3 mmol) and TFA (1 mL) in DCM was stirred at room temperature for 2 h, then concentrated under reduced pressure to give (1S,2R,3S)-2-methyl-3-(1-methyl-1H-imidazol-4-yl) cyclopropane-1-carboxylic acid as an oil, which was used directly in the next step without further purification. LCMS (ESI): m/z [M+H]⁺ calc'd for C₉H₁₂N₂O₂ 180.1; found 181.3.

### Step 4.

To a mixture of (6³S,3S,4S, Z)-4-amino-3-ethoxy-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2 (4,2)-thiazola-1 (5,3)-indola-6 (1,3)-pyridazinacycloundecaphane-5,7-dione (122 mg, 0.18 mmol) and tert-butyl (1S,2R,3S)-2-methyl-3-(1-methyl-1H-imidazol-4-yl) cyclopropane-1-carboxylate (65 mg, 0.36 mmol) in DMF (10 mL) at room temperature was added DIPEA (467 mg, 3.6 mmol) and HATU (76 mg, 0.2 mmol) in portions. The mixture was irradiated under microwave radiation for 2 h at room temperature. The mixture was extracted with EtOAc (2×20 mL) and the combined organic layers were washed with H₂O (3×20 mL), dried over anhydrous Na₂SO₄ and filtered. The filtrate was concentrated under reduced pressure and the residue was purified by preparative-HPLC to give (1R,2S,3R)—N-((6³S,3S,4S,Z)-3-ethoxy-1¹-ethyl-1²-(2-((S)-1-methoxyethyl) pyridin-3-yl)-10, 10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methyl-3-(1-methyl-1H-imidazol-4-yl) cyclopropane-1-carboxamide (34 mg, 21%, diastereomer of unknown absolute configuration RT=1.00 min) as a solid and (1R,2S,3R)—N-((6³S,3S,4S,Z)-3-ethoxy-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2 (4,2)-thiazola-1 (5,3)-indola-6 (1,3)-pyridazinacycloundecaphane-4-yl)-2-methyl-3-(1-methyl-1H-imidazol-4-yl) cyclopropane-1-carboxamide (36 mg, 22%, single diastereomer of unknown absolute configuration RT=1.07 min) as a solid. Data for first diastereomer (RT=1.00 min): LCMS (ESI): m/z [M+H]⁺ calc'd for C₄₅H₅₆N₈O₆S 836.4; found 837.1; ¹H NMR (400 MHZ, CD₃OD) δ 8.74-8.69 (m, 1H), 8.63-8.60 (m, 1H), 7.86-7.80 (m, 1H), 7.73-7.68 (m, 1H), 7.63 (s, 1H), 7.56-7.45 (m, 3H), 6.94 (s, 1H), 6.01 (s, 1H), 5.04-5.01 (m, 1H), 4.50-4.41 (m, 1H), 4.38-4.33 (m, 1H), 4.29-4.20 (m, 2H), 4.15-4.06 (m, 1H), 3.76-3.73 (m, 2H), 3.72-3.68 (m, 3H), 3.66-3.58 (m, 2H), 3.32-3.27 (m, 3H), 3.05-2.95 (m, 1H), 2.86-2.78 (m, 1H), 2.66-2.61 (m, 1H), 2.49-2.41 (m, 1H), 2.24-2.16 (m, 1H), 2.11-2.06 (m, 1H), 2.02-1.92 (m, 1H), 1.87-1.77 (m, 1H), 1.69-1.52 (m, 3H), 1.46-1.41 (m, 3H), 1.38-1.15 (m, 11H), 1.04-0.81 (m, 13H), 0.50 (s, 3H). Data for second diastereomer (RT=1.07 min): LCMS (ESI): m/z [M+H]⁺ calc'd for C₄₅H₅₆N₈O₈S 836.4; found 837.1; ¹H NMR (400 MHZ, CD₃OD) δ 8.74-8.67 (m, 1H), 8.63-8.60 (m, 1H), 7.86-7.80 (m, 1H), 7.73-7.68 (m, 1H), 7.63 (s, 1H), 7.56-7.45 (m, 3H), 6.94 (s, 1H), 6.01 (s, 1H), 5.04-5.01 (m, 1H), 4.50-4.41 (m, 1H), 4.38-4.33 (m, 1H), 4.29-4.20 (m, 2H), 4.15-4.06 (m, 1H), 3.76-3.73 (m, 2H), 3.72-3.68 (m, 3H), 3.66-3.58 (m, 2H), 3.32-3.27 (m, 3H), 3.05-2.95 (m, 1H), 2.86-2.78 (m, 1H), 2.66-2.61 (m, 1H), 2.49-2.41 (m, 1H), 2.24-2.16 (m, 1H), 2.11-2.06 (m, 1H), 2.02-1.92 (m, 3H), 1.87-1.77 (m, 3H), 1.68-1.53 (m, 4H), 1.46-1.41 (m, 11H), 1.38-1.15 (m, 3H), 1.04-0.81 (m, 10H), 0.50 (s, 3H).

### Biological Assays

All compounds herein exhibit an IC50 of 2 μM or less in an AsPC-1 (K-Ras G12D) PERK potency assay and/or a Capan-1 (K-Ras G12V) PERK potency assay.

Potency Assay: pERK

The purpose of this assay was to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: this protocol may be executed substituting other cell lines to characterize inhibitors of other RAS variants, including, for example, AsPC-1 (K-Ras G12D), Capan-1 (K-Ras G12V), NCI-H1355 (K-Ras G13C), Hs 766T (K-Ras Q61H), NCI-H2347 (N-Ras Q61R), or SK-MEL-30 (N-Ras Q61K).

NCI-H358 cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On day of assay, 40 nl of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μl lysis buffer, with shaking at 600 RPM at room temperature. Lysate (10 μl) was transferred to a 384-well Opti-plate (PerkinElmer) and 5 μl acceptor mix was added. After a 2-hour incubation in the dark, 5 μl donor mix was added, plate was sealed and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μl tris buffered saline (TBS) and fixed for 15 minutes with 150 μl 4% paraformaldehyde in TBS. Fixed cells were washed 4 times for 5 minutes with TBS containing 0.1% Triton X-100 (TBST) and then blocked with 100 μl Odyssey blocking buffer (LI-COR) for 60 minutes at room temperature. Primary antibody (PERK, CST-4370, Cell Signaling Technology) was diluted 1:200 in blocking buffer, and 50 μl was added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li—COR Odyssey CLx Imager. Analysis of raw data was carried out in Excel (Microsoft) and Prism (GraphPad). Signal was plotted vs. the decadal logarithm of compound concentration, and IC50 was determined by fitting a 4-parameter sigmoidal concentration response model.

### Compound A, a Representative Inhibitor of the Present Invention, Drives Regressions of KRAS^{G12D} Tumors in Vivo

Methods: Effects of Compound A on tumor cell growth in vivo were evaluated in the human pancreatic adenocarcinoma HPAC KRAS^{G12D/wt} xenograft model using female BALB/c nude mice (6-8 weeks old). Mice were implanted with HPAC tumor cells in PBS (3×10⁶ cells/mouse) subcutaneously in the flank. Once tumors reached an average size of ˜150 mm³, mice were randomized to treatment groups to start the administration of test articles or vehicle. Compound A was administered by oral gavage once every other day (po q2d). Body weight and tumor volume (using calipers) was measured twice weekly until study endpoints.

Results: Single-agent Compound A administered at 50 mg/kg po and 100 mg/kg po every other day led to complete regression of all tumors in each group (complete regression defined as >85% tumor regression from baseline) at the end of treatment (Day 38 after treatment started) in the HPAC CDX model with heterozygous KRAS^{G12D} (FIG. 1A). The anti-tumor activity of both tested doses of Compound A was statistically significant compared with control group (*** p<0.001, ordinary One-way ANOVA with multiple comparisons via a post-hoc Tukey's test).

### Compound A, a Representative Inhibitor of the Present Invention, Regulates RAS Pathway and Drives Regressions of KRAS^{G12V} Tumors in Vivo

Methods: Effects of Compound A on blood and tumor pharmacokinetics (PK), pharmacodynamics (PD), and tumor cell growth were evaluated in vivo in the human non-small cell lung cancer (NSCLC) NCI-H441 KRAS^{G12V/wt} xenograft model using female BALB/c nude mice (6-8 weeks old). Mice were implanted with NCI-H441 tumor cells (2×10⁶ cells/mouse) in 50% media, 50% Matrigel, subcutaneously in the flank. For PK/PD, animals were grouped out when tumors were ˜400 mm³ and animals were treated with a single dose of Compound A at 10, 25 or 50 mg/kg by oral gavage. For PK/PD n=3 measurements per timepoint.

Once tumors reached an average size of ˜155 mm³, mice were randomized to treatment groups to start the administration of test articles or vehicle. In NCI-H441 Compound A was administered by oral gavage once daily (po qd) at 10 or 25 mg/kg. Body weight and tumor volume (using calipers) was measured twice weekly until study endpoints.

Results: Pharmacokinetics were analyzed based on total concentration (nM) of Compound A in tumors or blood, following a single oral gavage dose of Compound A at 10, 25 or 50 mg/kg, monitored through 72 hours following dose. Compound A exhibited dose-dependent exposure in blood and tumor samples. Compound A treated at 25 mg/kg or 50 mg/kg doses was detectable in tumors through 72 hours following treatment (FIG. 1B). PK from naïve animals treated with a single dose of Compound A delivered at 10 mg/kg demonstrates maximum exposure of at 2 hours (FIG. 1C). Tumor DUSP6 demonstrates modulation of DUSP6, a marker of RAS pathway activity for 72 hours following single dose administration (FIG. 1C).

Single-agent Compound A administered to NCI-H441 tumor bearing animals, treated at 10 mg/kg po qd led to regressions (reductions in tumor volume >10% from initial) in all animals. Treated at 25 mg/kg po qd, Compound A led to complete regression of all tumors (complete regression defined as >85% tumor regression from baseline) at the end of treatment (Day 38 after treatment started) in the NCI-H441 CDX model with heterozygous KRAS^{G12V} (FIG. 1D, FIG. 1E). The anti-tumor activity of Compound A was statistically significant compared with control group (*** p<0.0001, ordinary One-way ANOVA with multiple comparisons via a post-hoc Tukey's test). Treatments were well tolerated by body weight measurements (FIG. 1F).

### Compound A, a Representative Inhibitor of the Present Invention, Drives Regressions of KRAS^{G12V} Pancreatic Ductal Adenocarcinoma and Colorectal Tumors in Vivo

Methods: Effects of Compound A on tumor cell growth in vivo were evaluated in the human pancreatic adenocarcinoma Capan-2 KRAS^{G12V/wt} and colorectal SW403 KRAS^{G12V/wt} xenograft models using female BALB/c nude mice (6-8 weeks old). Mice were implanted with Capan-2 tumor cells (4×106 (media/Matrigel) cells/mouse), or SW403 tumor cells (1×10⁷ cells/mouse) in 50% PBS, 50% Matrigel, subcutaneously in the flank. Once tumors reached an average size of ˜160-170 mm³, mice were randomized to treatment groups to start the administration of test articles or vehicle. In Capan-2, Compound A was administered by oral gavage once daily (po qd) at 10 or 25 mg/kg. In SW403 CDX, Compound A was administered by oral gavage once daily (po qd) at 25 mg/kg or every other day (q2d) at 50 mg/kg. Body weight and tumor volume (using calipers) was measured twice weekly until study endpoints.

Results: Single-agent Compound A administered to Capan-2 tumor bearing animals, treated at 10 mg/kg po qd led to 5/8 regressions, and 25 mg·kg po qd led to 8/8 regressions at the end of treatment (Day 38 after treatment started) in the Capan-2 CDX model with heterozygous KRAS^{G12V} (FIG. 2A, FIG. 2B). The anti-tumor activity of Compound A was statistically significant compared with control group (** p<0.01, *** p<0.0001, ordinary One-way ANOVA with multiple comparisons via a post-hoc Tukey's test). Treatments were well tolerated by body weight measurements (FIG. 2C). In SW403 CDX model, single-agent Compound A administered at 25 mg/kg po daily or 50 mg/kg po q2d led to significant tumor growth inhibition in all tumors through the end of treatment (Day 35 after treatment started). Compound A treatment at 25 mg/kg po qd drove regressions in 8/8 tumors, and 2/8 CR's (FIG. 2D, FIG. 2E). Compound A treatment at 50 mg/kg po q2d drove regressions in 8/8 tumors. The anti-tumor activity of Compound A was statistically significant compared with control group (*** p<0.0001, ordinary One-way ANOVA with multiple comparisons via a post-hoc Tukey's test). Treatments were well tolerated by body weight measurements (FIG. 2F).

### Compound A Exhibits Potent in Vivo Inhibition of Multiple RAS-Driven Cancer Cell Lines

Methods: Potency of in vitro cell proliferation inhibition of Capan-1 (KRAS^{G12V}), AsPC-1 (KRAS^{G12D}), HCT116 (KRAS^{G13D}), SK-MEL-30 (NRAS^{Q61K}), NCI-H1975 (EGFR^{T790M/L858R}), and A375 (BRAF^{V600E}) cells exposed to Compound A for 120 hours. Data represent the mean of multiple experiments. Cells were seeded in growth medium in 384-well assay plates and incubated overnight in a humidified atmosphere of 5% CO₂ at 37° C. The following day, cells were exposed to a 9-concentration 3-fold serial dilution of Compound A at a starting assay concentration of 1 μM (or 10 μM for A375). After 5 days of incubation, CellTiter-Glo® 2.0 Reagent was added to assay plates and luminescence measured. Data were normalized to the mean signal of DMSO-treated cells, and IC50 values were estimated using a four-parameter concentration response model.

Results: Compound A inhibited cell proliferation in RAS-driven lines (FIG. 3). IC50 for RAS-Driven cancer cell lines as follows: Capan-1 (KRAS^{G12V})=1 nM, AsPC-1 (KRAS^{G12D})=3 nM, HCT116 (KRAS^{G13D})=27 nM, SK-MEL-30 (NRAS^{Q61K})=13 nM, NCI-H1975 (EGFR^{T790M/L858R})=1 nM. RAS WT-Independent cell line A375 (BRAF^{V600E}) was not sensitive to Compound A treatment with IC50 >8700 nM.

### Compound A, a Representative Inhibitor of the Present Invention, Drives Regressions of KRAS^{G12D} Tumors in Vivo

Methods: Effects of Compound A on tumor cell growth in vivo were evaluated in the human pancreatic adenocarcinoma HPAC KRAS^{G12D/wt} and colorectal GP2d KRAS^{G12D/wt} xenograft models using female BALB/c nude mice (6-8 weeks old). Mice were implanted with HPAC tumor cells (3×10⁶ cells/mouse), or GP2d tumor cells (2×10⁶ cells/mouse) in 50% PBS, 50% Matrigel, subcutaneously in the flank. Once tumors reached an average size of ˜150 mm³, mice were randomized to treatment groups to start the administration of test articles or vehicle. Compound A was administered by oral gavage once daily (po qd) at 25 mg/kg. Body weight and tumor volume (using calipers) was measured twice weekly until study endpoints.

Results: Single-agent Compound A administered at 25 mg/kg po daily led to complete regression of all tumors (complete regression defined as >85% tumor regression from baseline) at the end of treatment (Day 38 after treatment started) in the HPAC CDX model with heterozygous KRAS^{G12D} (FIG. 4A, FIG. 4B). The anti-tumor activity of Compound A was statistically significant compared with control group (*** p<0.0001, ordinary One-way ANOVA with multiple comparisons via a post-hoc Tukey's test). Treatments were well tolerated by body weight measurements (FIG. 4C). In Gp2d CDX model, single-agent Compound A administered at 25 mg/kg po daily led to significant tumor growth inhibition in all tumors through the end of treatment (Day 35 after treatment started) (FIG. 4D, FIG. 4E). The anti-tumor activity of Compound A was statistically significant compared with control group (*** p<0.0001, ordinary One-way ANOVA with multiple comparisons via a post-hoc Tukey's test). Treatments were well tolerated by body weight measurements (FIG. 4F).

### Compound A Down-Regulates Immune Checkpoint Proteins in NCI-H358, SW900, and Capan-2 Cells in Vitro

Methods: To assess the effect of Compound A on checkpoint molecule expression in vitro, NCI-H358, SW900 or Capan-2 cells (5e4 cells/well) were seeded in a 96-well plate and after 24 hours treated with a five-fold dilution of Compound A in the presence of 250 μg/ml IFNγ. The plates were incubated for 48 hours at 37° C. and 5% CO₂. The cells were detached with 0.25% Trypsin, incubated for 15 minutes in PBS containing Fixable Blue Dead Cell Stain (Invitrogen) and subsequently incubated with FITC anti-human CD₂₇₄ (PD-L1), PerCP/Cyanine5.5 anti-human CD₁₅₅ (PVR) and Brilliant Violet 605 anti-human CD₇₃ (Biolegend) for 30 minutes on ice. The cells were washed twice with staining buffer (PBS/2% FCS) before flow cytometric acquisition on a Cytek Aurora instrument. The analysis was performed using the SpectroFlo and FlowJo v10 software.

Results: Compound A produced a concentration-dependent 2- to 5-fold decrease of PD-L1, PVR and CD₇₃ on NCI-H358 (FIG. 5A), SW900 (FIG. 5B), or Capan-2 (FIG. 5C) cells in vitro. Down-regulation of these proteins is predicted to transform the immuno-suppressive tumor immune microenvironment in favor of anti-tumor immunity (Rothlin et al JITC 2020).

### Compound a, a KRAS^{MULTI}(ON) Inhibitor Disclosed Herein, is Active Against RAS Oncogene Switching Mutations

FIG. 6A is a heatmap representing cellular RAS/RAF disruption assay results regarding various KRAS mutations in the presence of different RAS inhibitors (Compound A, a KRAS^{MULTI}(ON) inhibitor disclosed herein, and KRAS^{G12C}(OFF) inhibitors MRTX849 and AMG 510).

Plasmids expressing nanoluciferase-tagged mutant KRAS4B and halo-tagged RAF1 (residues 51-149) were co-transfected into U2OS cells and incubated for 24 hours. Plasmids encoding the relevant mutation were generated by New England Biolabs Q5 site-directed mutagenesis. Transfected cells were reseeded at 25000 cells/well in 96-well plates in assay media (OptiMEM+4% FBS+100 nM HaloTag NanoBRET 618 Ligand) and incubated overnight. Promega Vivazine Nano-Glo substrate was added according to manufacturer's instructions. Compounds were added at concentrations ranging from 0 to 10 μM and incubated for 1 hour. The luminescence signal was measured at 460 nm and 618 nm and the BRET ratio was calculated as the 618 nm signal divided by the 460 nm signal. The BRET ratios were fit to a standard sigmoidal dose response function and the IC50 values were used to calculate the Log 2 (Fold-Change) relative to KRASG12C. FIG. 6B shows the IC50 value associated with each colored bar of the heatmap.

The following four assays may be conducted to further characterize properties of compounds of the present invention.

### Determination of Cell Viability in RAS Mutant Cancer Cell Lines Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay is to determine the effects of test compounds on the proliferation of three human cancer cell lines (NCI-H358 (K-Ras G12C), AsPC-1 (K-Ras G12D), Capan-1 (K-Ras G12V)) over a 5-day treatment period by quantifying the amount of ATP present at endpoint using the CellTiter-Glo® 2.0 Reagent (Promega).

Cells are seeded at 250 cells/well in 40 μl of growth medium in 384-well assay plates and incubated overnight in a humidified atmosphere of 5% CO₂ at 37° C. On the day of the assay, 10 mM stock solutions of test compounds are first diluted into 3 mM solutions with 100% DMSO. Well-mixed compound solutions (15 μl) are transferred to the next wells containing 30 μl of 100% DMSO, and repeated until a 9-concentration 3-fold serial dilution is made (starting assay concentration of 10 μM). Test compounds (132.5 nl) are directly dispensed into the assay plates containing cells. The plates are shaken for 15 seconds at 300 rpm, centrifuged, and incubated in a humidified atmosphere of 5% CO₂ at 37° C. for 5 days. On day 5, assay plates and their contents are equilibrated to room temperature for approximately 30 minutes. CellTiter-Glo® 2.0 Reagent (25 μl) is added, and plate contents are mixed for 2 minutes on an orbital shaker before incubation at room temperature for 10 minutes. Luminescence may be measured using the PerkinElmer Enspire. Data may be normalized by the following: (Sample signal/Avg. DMSO)*100. The data may be fit using a four-parameter logistic fit.

Disruption of B-Raf Ras-Binding Domain (BRAF^{RBD}) Interaction with K-Ras by Compounds of the Invention

Note—The following protocol describes a procedure for monitoring disruption of K-Ras G12C (GMP-PNP) binding to BRAF^{RBD} by a compound of the invention. This protocol may also be executed substituting other Ras proteins or nucleotides.

The purpose of this biochemical assay is to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data may be reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a K-Ras: RAF complex are identified as those eliciting a decrease in the TR-FRET ratio relative to DMSO control wells.

### Determination of Cell Viability in RAS Mutant Cancer Cell Lines

### Protocol: CellTiter-Glo® Cell Viability Assay

Note—The following protocol describes a procedure for monitoring cell viability of KRAS mutant cancer cell lines in response to a compound of the invention. Other RAS isoforms may be employed, though the number of cells to be seeded will vary based on cell line used.

The purpose of this cellular assay is to determine the effects of test compounds on the proliferation of three human cancer cell lines (NCI-H358 (KRAS G12C), AsPC-1 (KRAS G12D), Capan-1 (KRAS G12V)) over a 5-day treatment period by quantifying the amount of ATP present at endpoint using the CellTiter-Glo® 2.0 Reagent (Promega).

Cells are seeded at 250 cells/well in 40 μl of growth medium in 384-well assay plates and incubated overnight in a humidified atmosphere of 5% CO₂ at 37° C. On the day of the assay, 10 mM stock solutions of test compounds are first diluted into 3 mM solutions with 100% DMSO. Well-mixed compound solutions (15 μl) are transferred to the next wells containing 30 μl of 100% DMSO, and repeated until a 9-concentration 3-fold serial dilution is made (starting assay concentration of 10 μM). Test compounds (132.5 nl) are directly dispensed into the assay plates containing cells. The plates are shaken for 15 seconds at 300 rpm, centrifuged, and incubated in a humidified atmosphere of 5% CO₂ at 37° C. for 5 days. On day 5, assay plates and their contents are equilibrated to room temperature for approximately 30 minutes. CellTiter-Glo® 2.0 Reagent (25 μl) is added, and plate contents are mixed for 2 minutes on an orbital shaker before incubation at room temperature for 10 minutes. Luminescence may be measured using the PerkinElmer Enspire. Data is normalized by the following: (Sample signal/Avg. DMSO)*100. The data may be fit using a four-parameter logistic fit.

### Determination of Oral Bioavailability

Oral bioavailability may be determined in BALB/c mice. Following intravenous (IV) bolus and oral gavage (PO) administration of a test compound, about 30 μL of whole blood samples are collected at designated time points into tubes containing K₂EDTA. The blood samples are centrifuged at 4600 rpm at 4° C. for about 5 minutes and plasma samples are stored at −80° C. prior to bioanalysis. Plasma samples are extracted by protein precipitation and analyzed by tandem mass spectrometry (LC MS/MS) on, for example, an API 5500 system using electrospray positive ionization.

All PK parameters may be derived from plasma concentration over time data with noncompartment analysis using WinNonlin. The bioavailability (F %) was estimated using the following equation:

$$F \% = \frac{{AUC}_{inf , PO}}{{AUC}_{inf , IV}} \cdot \frac{{Dose}_{IV}}{{Dose}_{PO}}$$

AUC_{inf,PO} is the area under the plasma concentration over time from time zero to infinity following
PO administration.
AUC_{inf,Iv} is the area under the plasma concentration over time from time zero to infinity following IV administration.
Dose_{Iv} is the total dose of IV administration
Dose_{PO} is the total dose of PO administration

In general, F % values of over 30% are preferred, with values over 50% being more preferred.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

### Numbered Embodiments

[1] A compound, or pharmaceutically acceptable salt thereof, having the structure of Formula I:

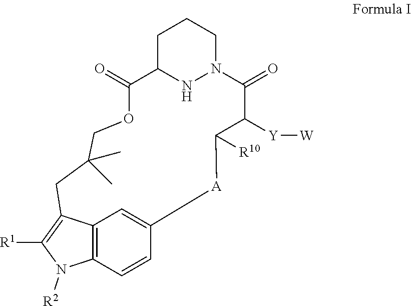

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
Y is

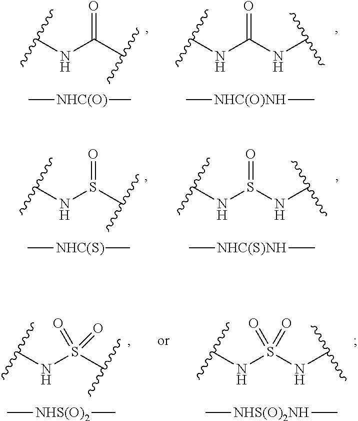

W is hydrogen, C₁-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl.
[2] The compound of paragraph [1], or pharmaceutically acceptable salt thereof, wherein R¹ is optionally substituted 6 to 10-membered aryl or optionally substituted 5 to 10-membered heteroaryl.
[3] The compound of paragraph [2], or pharmaceutically acceptable salt thereof, wherein R¹ is optionally substituted phenyl or optionally substituted pyridine.
[4] The compound of any one of paragraphs [1] to [3], or pharmaceutically acceptable salt thereof, wherein A is optionally substituted thiazole, optionally substituted triazole, optionally substituted morpholino, or phenyl.
[5] The compound of any one of paragraphs [1] to [3], or pharmaceutically acceptable salt thereof, wherein A is not an optionally substituted phenyl or benzimidazole.
[6] The compound of paragraph [5], or pharmaceutically acceptable salt thereof, wherein A is not hydroxyphenyl.
[7] The compound of any one of paragraphs [1] to [6], or pharmaceutically acceptable salt thereof, wherein the compound is not a compound of Table 2.
[8] The compound of any one of paragraphs [1] to [7], or pharmaceutically acceptable salt thereof, wherein the compound is not a compound of Table 3.
[9] The compound of any one of paragraphs [1] to [8], or pharmaceutically acceptable salt thereof, wherein Y is —NHC(O)— or —NHC(O)NH—.
[10] The compound of paragraph [9], or pharmaceutically acceptable salt thereof, having the structure of Formula II:

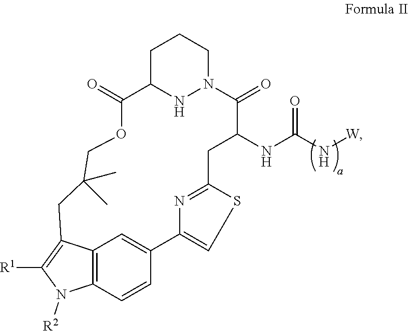

wherein a is 0 or 1.
[11] The compound of paragraph [10], or pharmaceutically acceptable salt thereof, having the structure of Formula II-1:

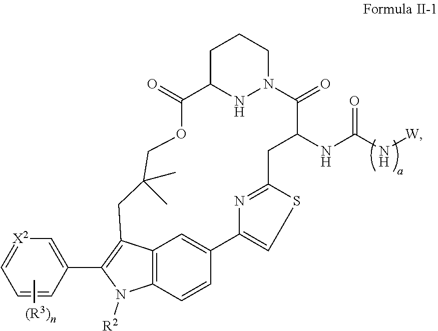

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[12] The compound of paragraph [11], or pharmaceutically acceptable salt thereof, having the structure of Formula II-2:

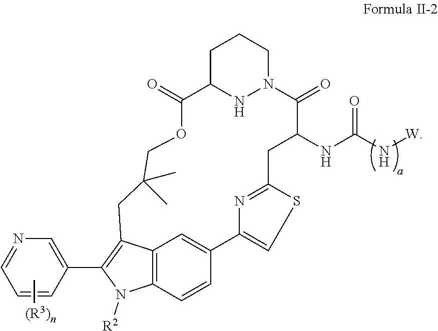

[13] The compound of paragraph [12], or pharmaceutically acceptable salt thereof, having the structure of Formula II-3:

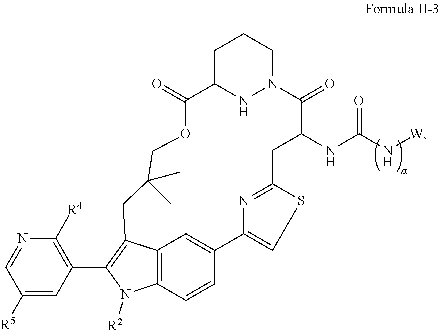

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[14] The compound of paragraph [13], or pharmaceutically acceptable salt thereof, having the structure of Formula II-4:

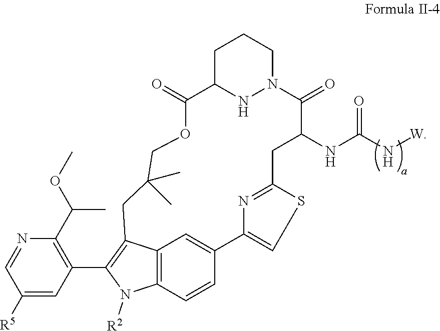

[15] The compound of paragraph [14], or pharmaceutically acceptable salt thereof, having the structure of Formula II-5:

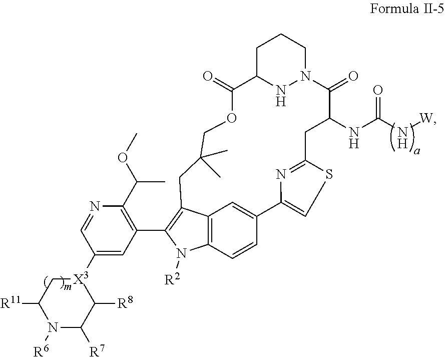

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[16] The compound of paragraph [15], or pharmaceutically acceptable salt thereof, having the structure of Formula II-6:

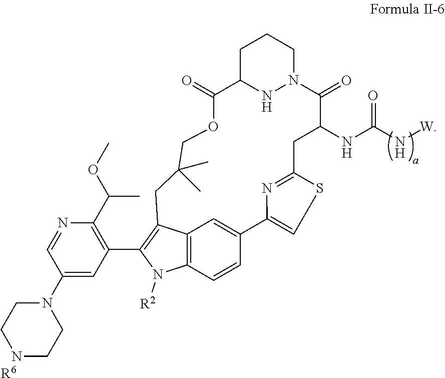

[17] The compound of paragraph [15], or pharmaceutically acceptable salt thereof, having the structure of Formula II-7:

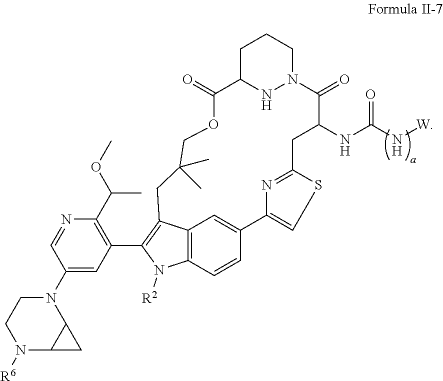

[18] The compound of paragraph [16] or [17], wherein R⁶ is methyl.
[19] The compound of paragraph [15], or pharmaceutically acceptable salt thereof, having the structure of Formula II-8 or Formula II-9:

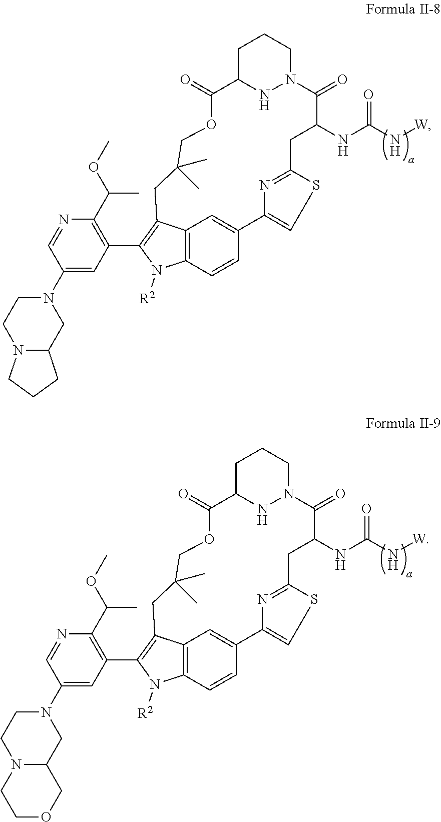

[20] The compound of paragraph [9], or pharmaceutically acceptable salt thereof, having the structure of Formula III:

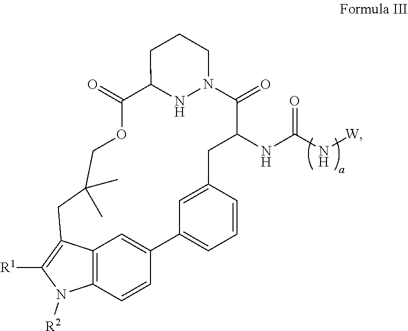

wherein a is 0 or 1.
[21] The compound of paragraph [20], or pharmaceutically acceptable salt thereof, having the structure of Formula III-1:

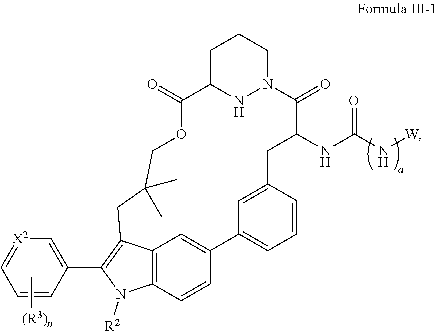

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[22] The compound of paragraph [21], or pharmaceutically acceptable salt thereof, having the structure of Formula III-2:

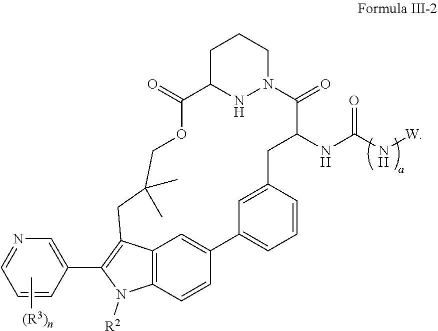

[23] The compound of paragraph [22], or pharmaceutically acceptable salt thereof, having the structure of Formula III-3:

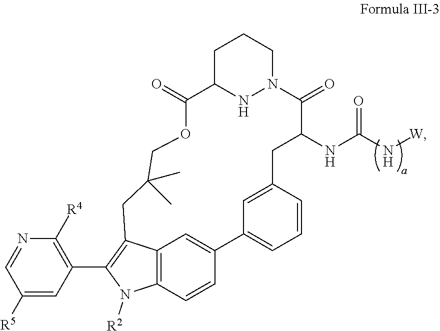

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[24] The compound of paragraph [23], or pharmaceutically acceptable salt thereof, having the structure of Formula III-4:

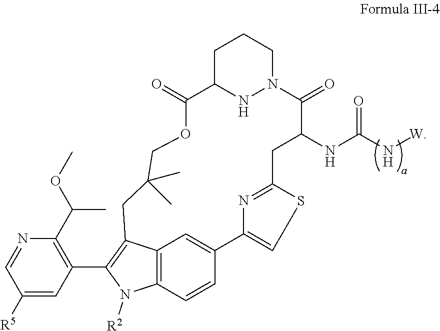

[25] The compound of paragraph [24], or pharmaceutically acceptable salt thereof, having the structure of Formula III-5:

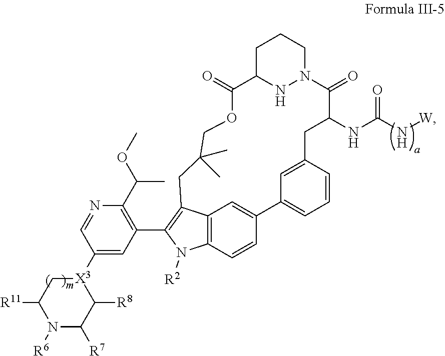

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[26] The compound of paragraph [25], or pharmaceutically acceptable salt thereof, having the structure of Formula III-6:

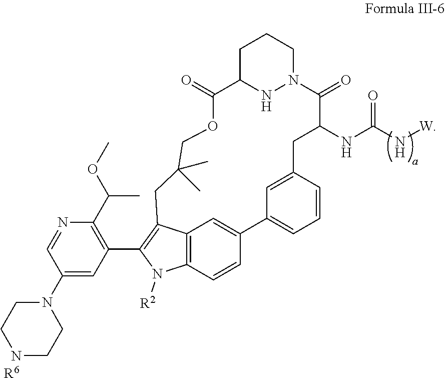

[27] The compound of paragraph [25], or pharmaceutically acceptable salt thereof, having the structure of Formula III-7:

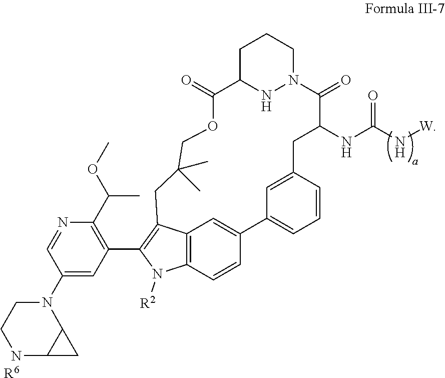

[28] The compound of paragraph [26] or [27], wherein R⁶ is methyl.
[29] The compound of paragraph [25], or pharmaceutically acceptable salt thereof, having the structure of Formula III-8 or Formula III-9:

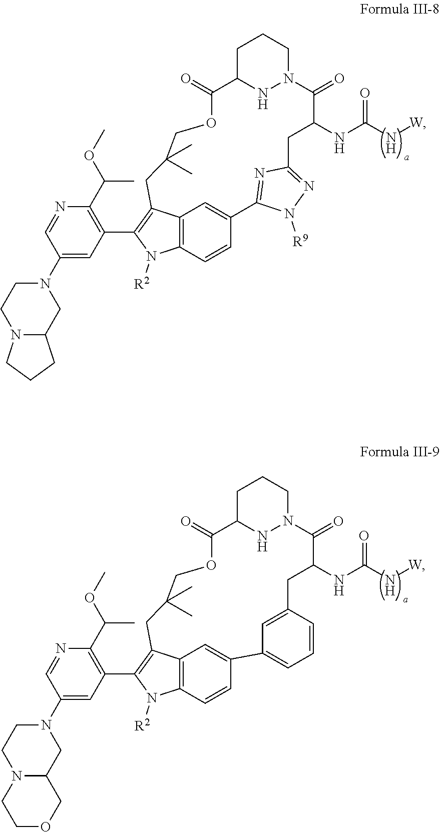

[30] The compound of paragraph [9], or pharmaceutically acceptable salt thereof, having the structure of Formula IV:

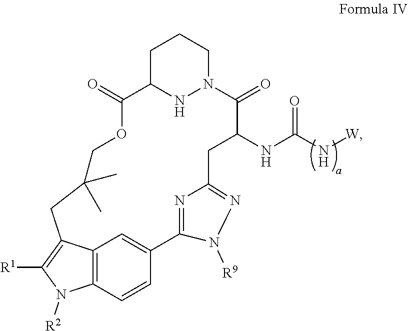

wherein R⁹ is H or C₁-C₆ alkyl; and
a is 0 or 1.
[31] The compound of paragraph [30], or pharmaceutically acceptable salt thereof, having the structure of Formula IV-1:

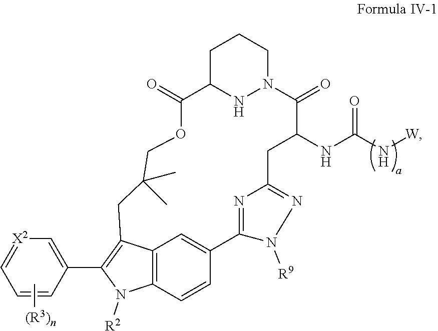

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[32] The compound of paragraph [31], or pharmaceutically acceptable salt thereof, having the structure of Formula IV-2:

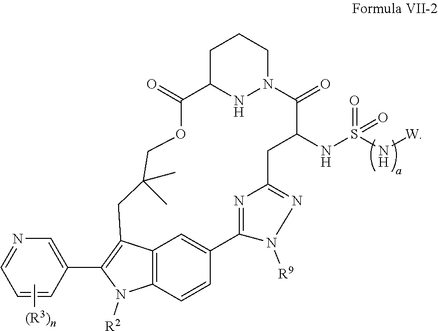

[33] The compound of paragraph [32], or pharmaceutically acceptable salt thereof, having the structure of Formula IV-3:

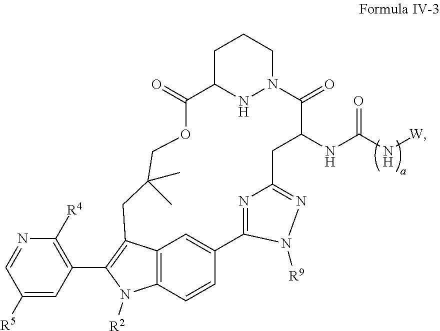

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[34] The compound of paragraph [33], or pharmaceutically acceptable salt thereof, having the structure of Formula IV-4:

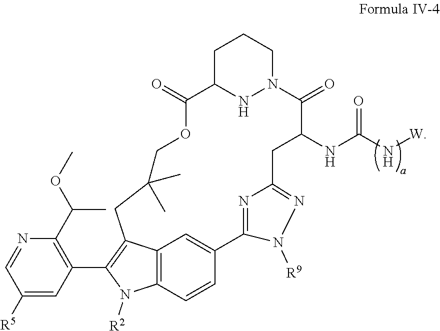

[35] The compound of paragraph [34], or pharmaceutically acceptable salt thereof, having the structure of Formula IV-5:

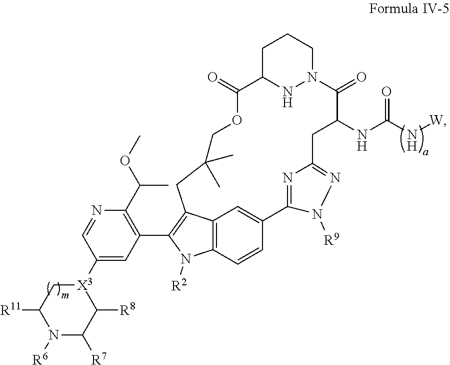

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[36] The compound of paragraph [35], or pharmaceutically acceptable salt thereof, having the structure of Formula IV-6:

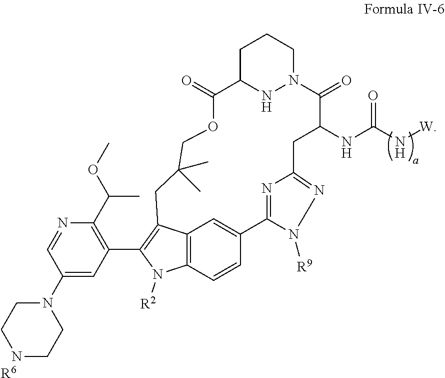

[37] The compound of paragraph [35], or pharmaceutically acceptable salt thereof, having the structure of Formula IV-7:

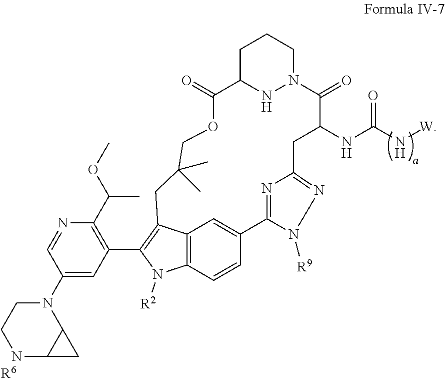

[38] The compound of paragraph [36] or [37], wherein R⁶ is methyl.
[39] The compound of paragraph [35], or pharmaceutically acceptable salt thereof, having the structure of Formula IV-8 or Formula IV-9:

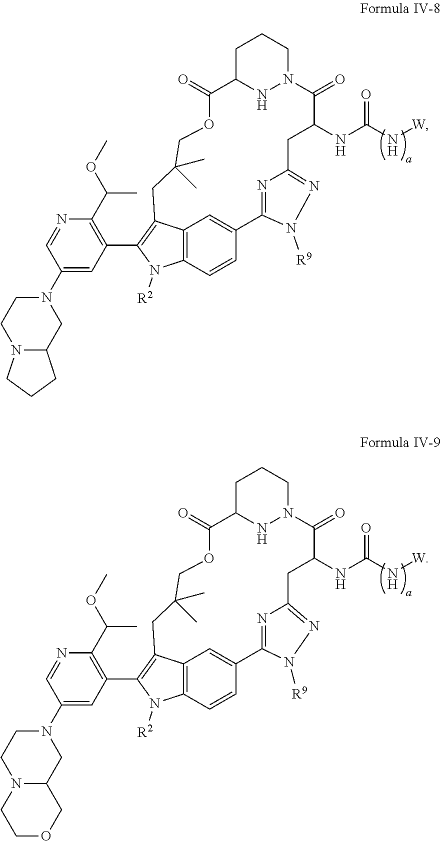

[40] The compound of any one of paragraphs [30] to [39], or pharmaceutically acceptable salt thereof, wherein R⁹ is methyl.
[41] The compound of any one of paragraphs [1] to [8], or pharmaceutically acceptable salt thereof, wherein Y is —NHS(O)₂— or —NHS(O)₂NH—.
[42] The compound of paragraph [41], or pharmaceutically acceptable salt thereof, having the structure of Formula V:

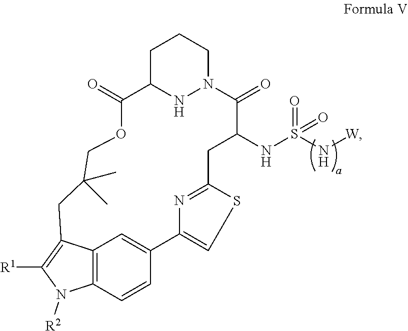

wherein a is 0 or 1.
[43] The compound of paragraph [42], or pharmaceutically acceptable salt thereof, having the structure of Formula V-1:

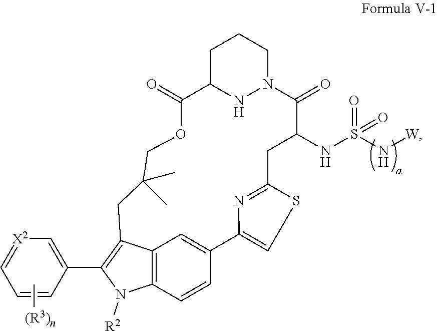

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[44] The compound of paragraph [43], or pharmaceutically acceptable salt thereof, having the structure of Formula V-2:

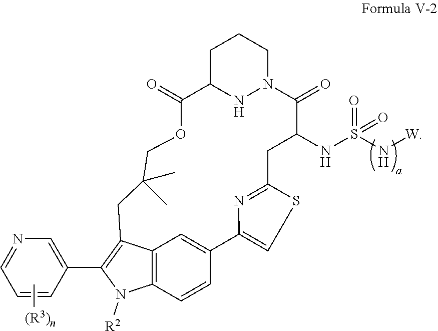

[45] The compound of paragraph [44], or pharmaceutically acceptable salt thereof, having the structure of Formula V-3:

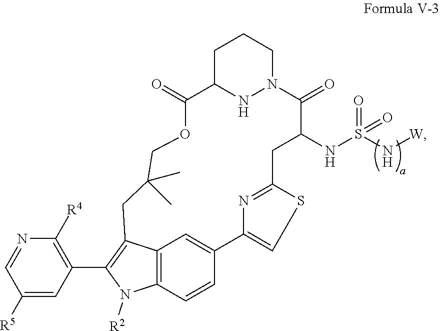

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[46] The compound of paragraph [45], or pharmaceutically acceptable salt thereof, having the structure of Formula V-4:

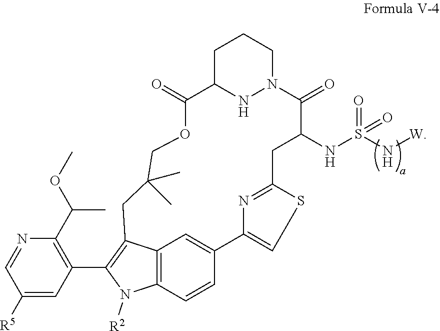

[47] The compound of paragraph [46], or pharmaceutically acceptable salt thereof, having the structure of Formula V-5:

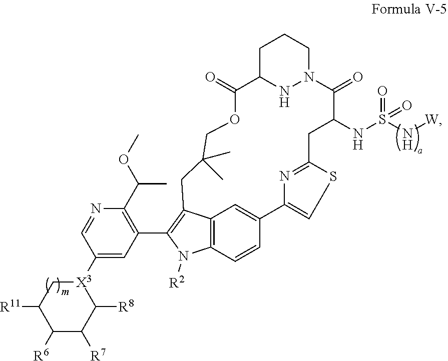

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[48] The compound of paragraph [41], or pharmaceutically acceptable salt thereof, having the structure of Formula VI:

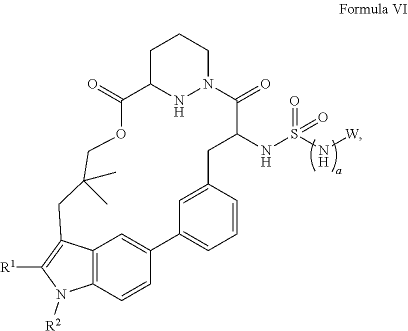

wherein a is 0 or 1.
[49] The compound of paragraph [48], or pharmaceutically acceptable salt thereof, having the structure of Formula VI-1:

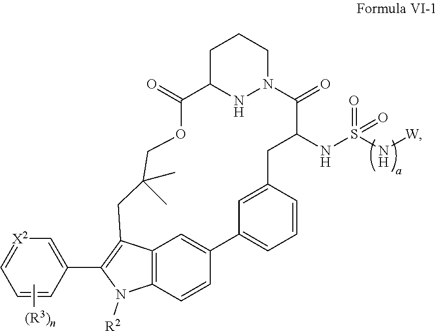

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[50] The compound of paragraph [49], or pharmaceutically acceptable salt thereof, having the structure of Formula VI-2:

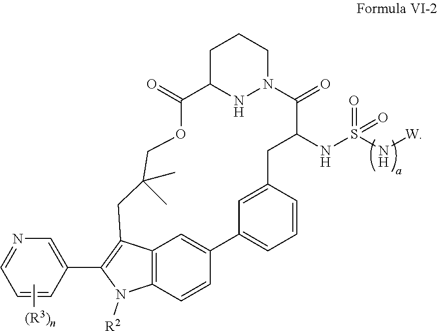

[51] The compound of paragraph [50], or pharmaceutically acceptable salt thereof, having the structure of Formula VI-3:

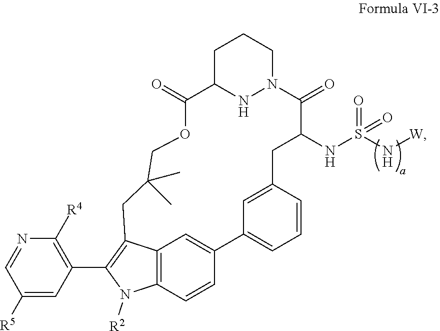

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[52] The compound of paragraph [51], or pharmaceutically acceptable salt thereof, having the structure of Formula VI-4:

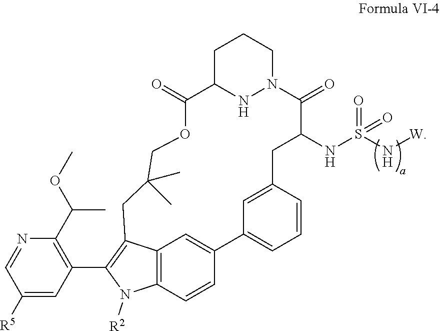

[53] The compound of paragraph [52], or pharmaceutically acceptable salt thereof, having the structure of Formula VI-5:

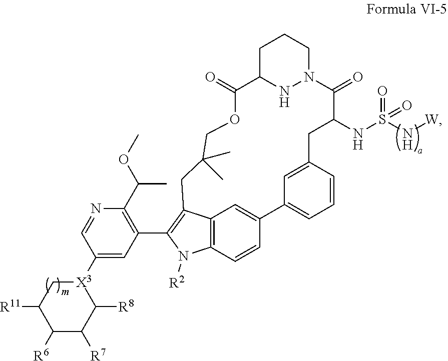

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[54] The compound of paragraph [41], or pharmaceutically acceptable salt thereof, having the structure of Formula VII:

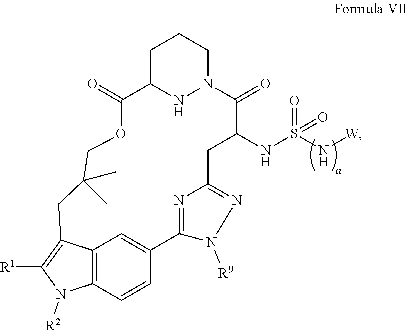

wherein R⁹ is H or C₁-C₆ alkyl; and
a is 0 or 1.
[55] The compound of paragraph [54], or pharmaceutically acceptable salt thereof, having the structure of Formula VII-1:

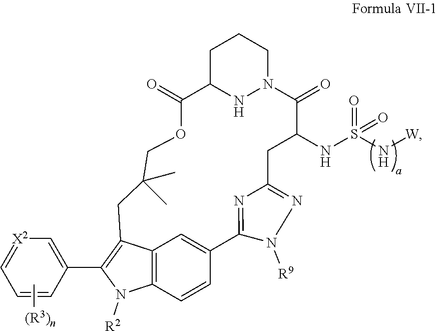

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[56] The compound of paragraph [55], or pharmaceutically acceptable salt thereof, having the structure of Formula VII-2:

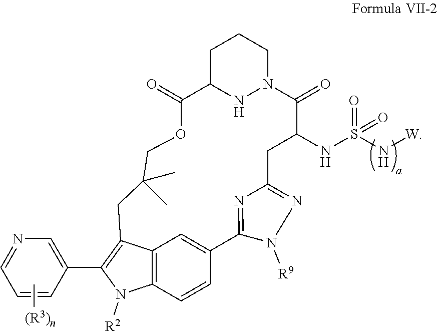

[57] The compound of paragraph [56], or pharmaceutically acceptable salt thereof, having the structure of Formula VII-3:

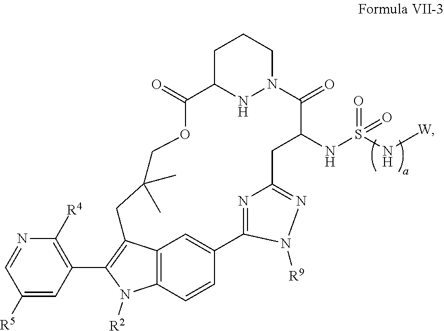

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[58] The compound of paragraph [57], or pharmaceutically acceptable salt thereof, having the structure of Formula VII-4:

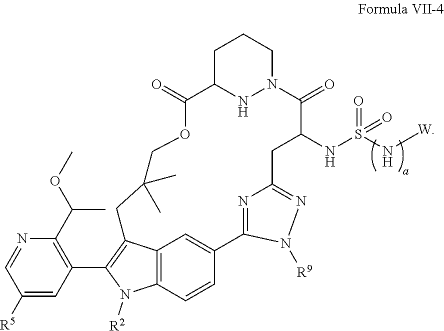

[59] The compound of paragraph [58], or pharmaceutically acceptable salt thereof, having the structure of Formula VII-5:

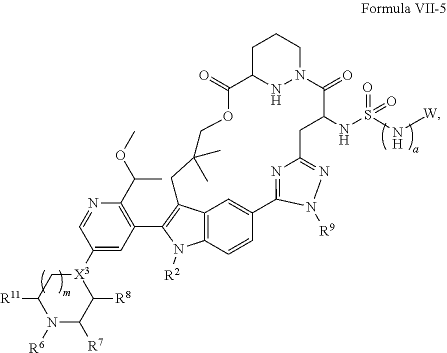

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[60] The compound of any one of paragraphs [54] to [59], or pharmaceutically acceptable salt thereof, wherein R⁹ is methyl.
[61] The compound of any one of paragraphs [1] to [8], or pharmaceutically acceptable salt thereof, wherein Y is —NHS (O)— or —NHS (O)NH—.
[62] The compound of paragraph [61], or pharmaceutically acceptable salt thereof, having the structure of Formula VIII:

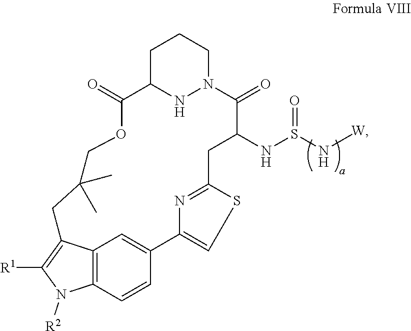

wherein a is 0 or 1.
[63] The compound of paragraph [62], or pharmaceutically acceptable salt thereof, having the structure of Formula VIII-1:

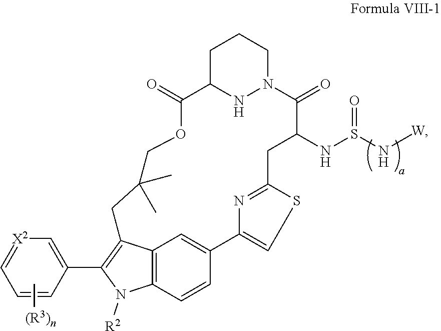

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[64] The compound of paragraph [63], or pharmaceutically acceptable salt thereof, having the structure of Formula VIII-2:

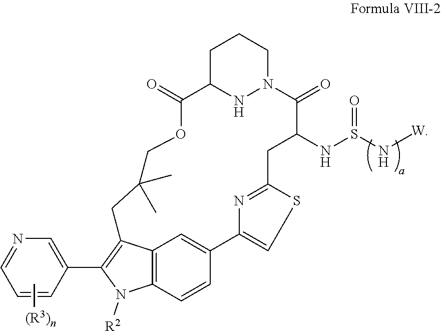

[65] The compound of paragraph [64], or pharmaceutically acceptable salt thereof, having the structure of Formula VIII-3:

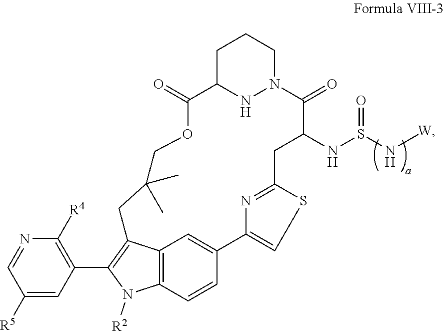

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[66] The compound of paragraph [65], or pharmaceutically acceptable salt thereof, having the structure of Formula VIII-4:

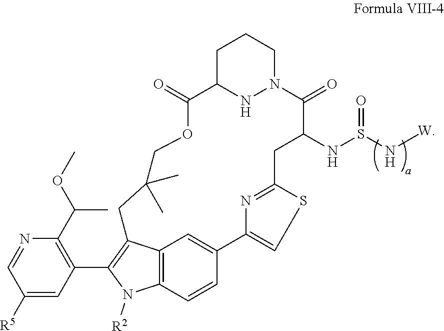

[67] The compound of paragraph [66], or pharmaceutically acceptable salt thereof, having the structure of Formula VIII-5:

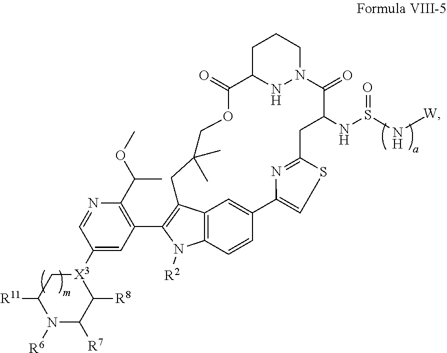

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[68] The compound of paragraph [61], or pharmaceutically acceptable salt thereof, having the structure of Formula IX:

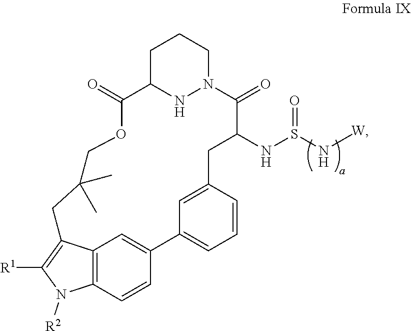

wherein a is 0 or 1.
[69] The compound of paragraph [68], or pharmaceutically acceptable salt thereof, having the structure of Formula IX-1:

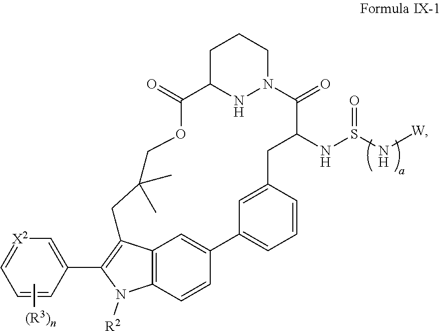

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[70] The compound of paragraph [69], or pharmaceutically acceptable salt thereof, having the structure of Formula IX-2:

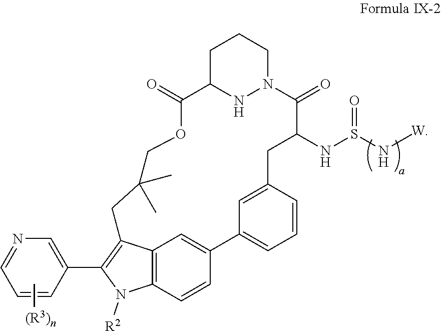

[71] The compound of paragraph [70], or pharmaceutically acceptable salt thereof, having the structure of Formula IX-3:

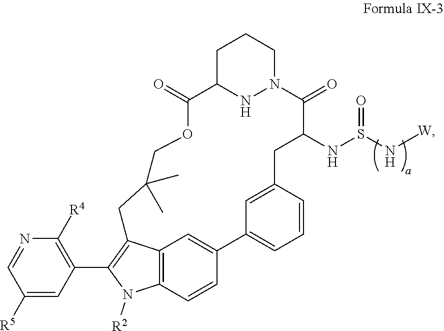

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[72] The compound of paragraph [71], or pharmaceutically acceptable salt thereof, having the structure of Formula IX-4:

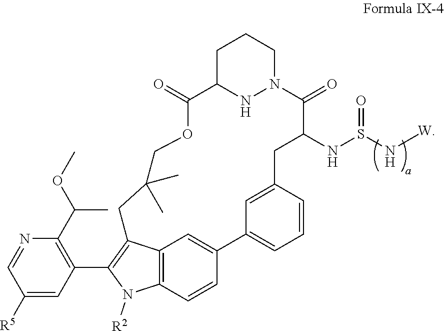

[73] The compound of paragraph [72], or pharmaceutically acceptable salt thereof, having the structure of Formula IX-5:

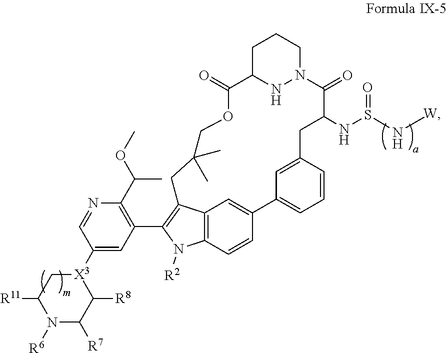

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[74] The compound of paragraph [61], or pharmaceutically acceptable salt thereof, having the structure of Formula X:

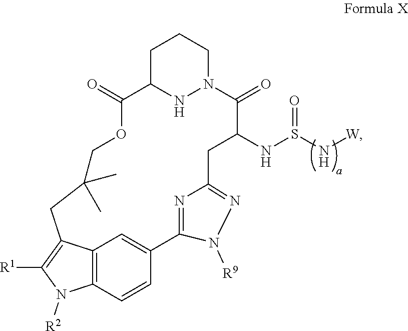

wherein R⁹ is H or C₁-C₆ alkyl; and
a is 0 or 1.
[75] The compound of paragraph [74], or pharmaceutically acceptable salt thereof, having the structure of Formula X-1:

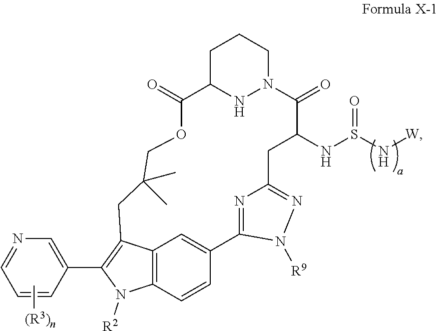

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl; and
n is an integer from 1 to 4.
[76] The compound of paragraph [75], or pharmaceutically acceptable salt thereof, having the structure of Formula X-2:

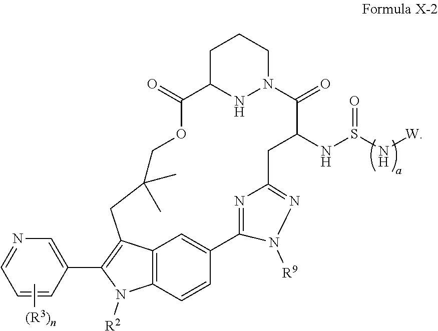

[77] The compound of paragraph [76], or pharmaceutically acceptable salt thereof, having the structure of Formula X-3:

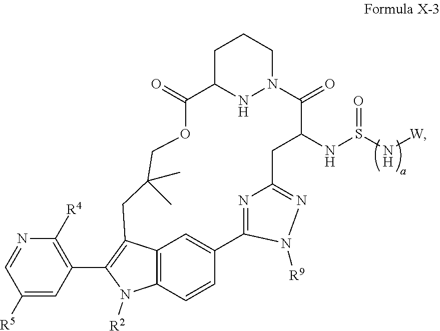

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl, optionally substituted 6 to 10-membered aryl, or optionally substituted 5 to 10-membered heteroaryl.
[78] The compound of paragraph [77], or pharmaceutically acceptable salt thereof, having the structure of Formula X-4:

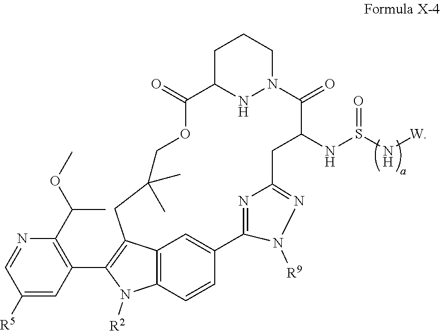

[79] The compound of paragraph [78], or pharmaceutically acceptable salt thereof, having the structure of Formula X-5:

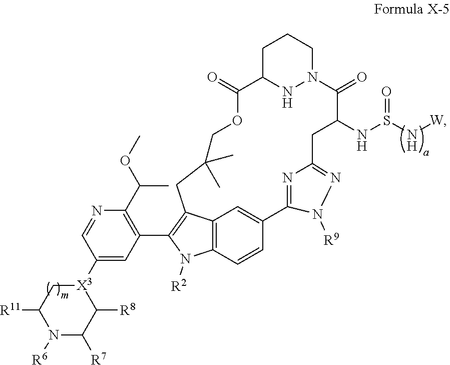

wherein X³ is N or CH;
m is 1 or 2;
R⁶, R⁷, R⁸, and R¹¹ are each independently selected from optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl.
[80] The compound of any one of paragraphs [74] to [79], or pharmaceutically acceptable salt thereof, wherein R⁹ is methyl.
[81] The compound of any one of paragraphs [10] to [40], [42] to [60], and [62] to [80], or pharmaceutically acceptable salt thereof, wherein a is 0.
[82] The compound of any one of paragraphs [10] to [40], [42] to [60], and [62] to [80], or pharmaceutically acceptable salt thereof, wherein a is 1.
[83] The compound of any one of paragraphs [1] to [82], or pharmaceutically acceptable salt thereof, wherein R² is optionally substituted C₁-C₆ alkyl.
[84] The compound of paragraph [83], or pharmaceutically acceptable salt thereof, wherein R² is selected from —CH₂CH₃ or —CH₂CF₃.
[85] The compound of any one of paragraphs [1] to [84], or pharmaceutically acceptable salt thereof, wherein W is C₁-C₄ alkyl.
[86] The compound of any one of paragraphs [1] to [84], or pharmaceutically acceptable salt thereof, wherein W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.
[87] The compound of any one of paragraphs [1] to [84], or pharmaceutically acceptable salt thereof, wherein W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.
[88] The compound of any one of paragraphs [1] to [84], or pharmaceutically acceptable salt thereof, wherein W is optionally substituted 3 to 10-membered heterocycloalkyl.
[89] The compound of paragraph [88], or pharmaceutically acceptable salt thereof, wherein W is selected from the following, or a stereoisomer thereof:

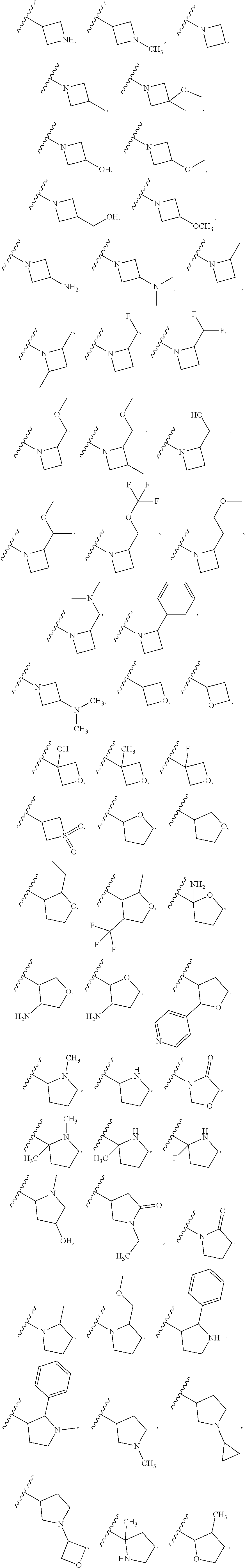

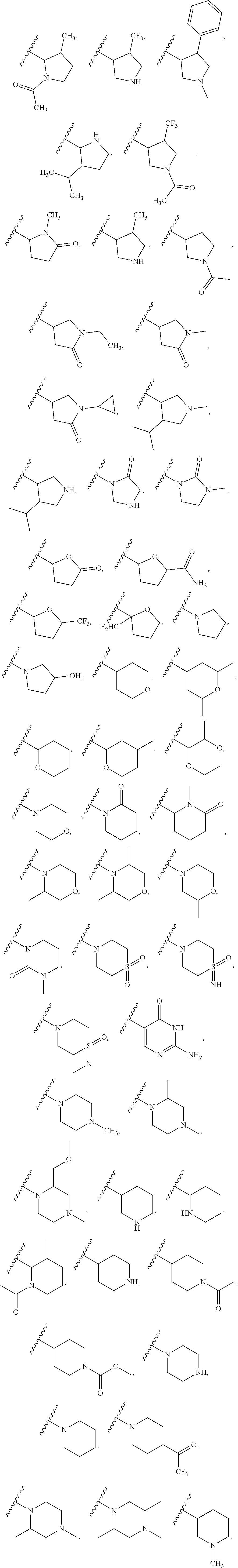

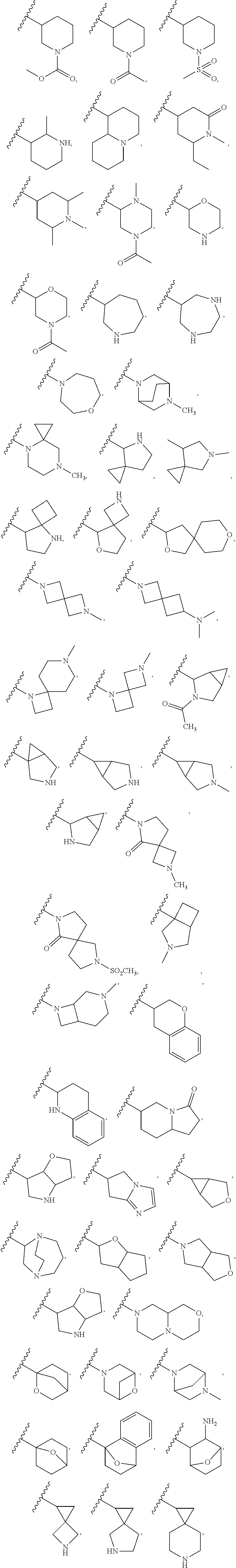

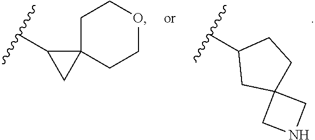 [SMILES: *C1CC12CCOCC2 ; *C1CCC2(CNC2)C1]

[90] The compound of any one of paragraphs [1] to [84], or pharmaceutically acceptable salt thereof, wherein W is optionally substituted 3 to 10-membered cycloalkyl.
[91] The compound of paragraph [90], or pharmaceutically acceptable salt thereof, wherein W is selected from the following, or a stereoisomer thereof:

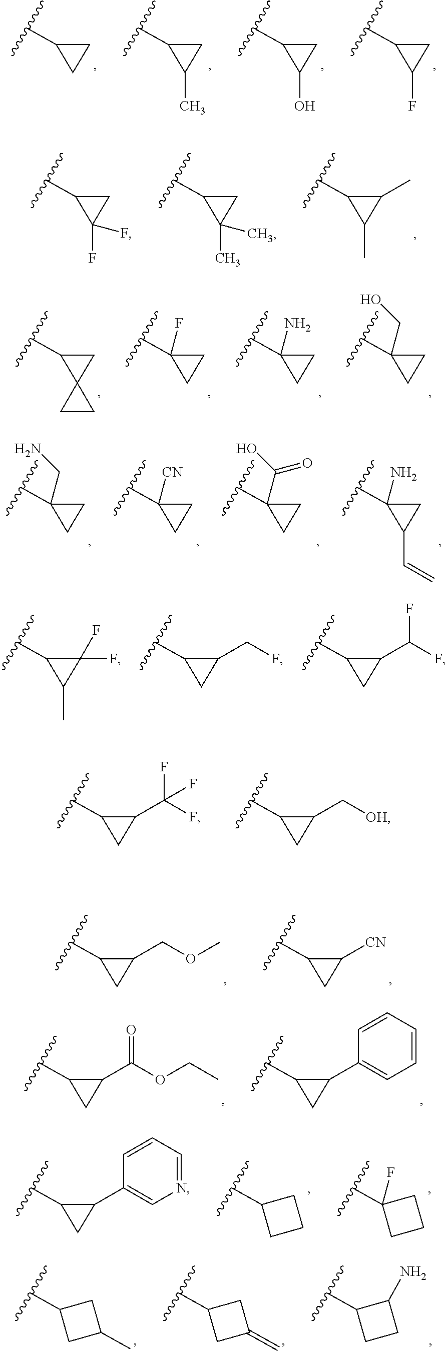

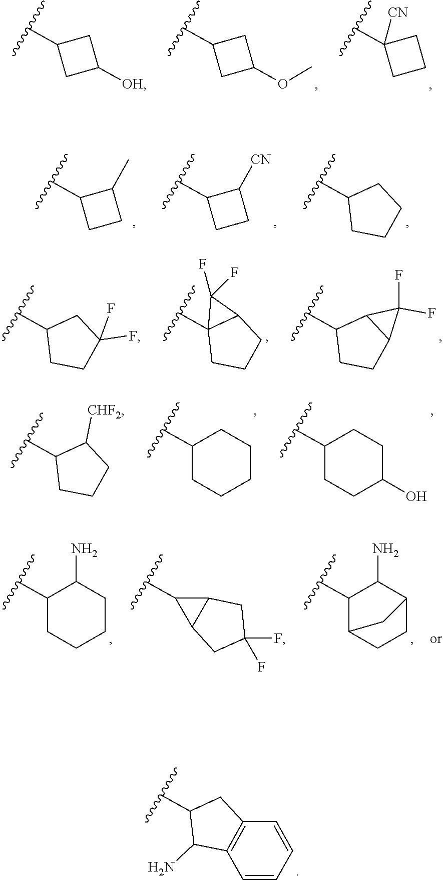

[92] The compound of any one of paragraphs [1] to [84], or pharmaceutically acceptable salt thereof, wherein W is optionally substituted 5 to 10-membered heteroaryl.
[93] The compound of paragraph [92], or pharmaceutically acceptable salt thereof, wherein W is selected from the following, or a stereoisomer thereof:

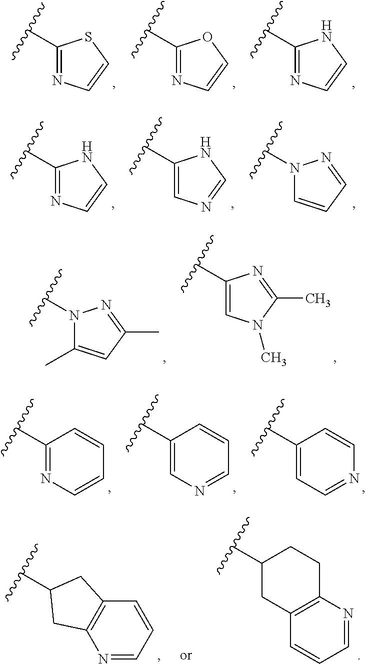

[94] The compound of any one of paragraphs [1] to [84], or pharmaceutically acceptable salt thereof, wherein W is optionally substituted 6 to 10-membered aryl.
[95] The compound of paragraph [94], or pharmaceutically acceptable salt thereof, wherein W is optionally substituted phenyl.
[96] A compound, or a pharmaceutically acceptable salt thereof, of Table 1.
[97] A compound, or a pharmaceutically acceptable salt thereof, of Table 1-1.
[98] A pharmaceutical composition comprising a compound, or a pharmaceutically acceptable salt thereof, of any one of paragraphs [1] to [97] and a pharmaceutically acceptable excipient.

1. A method of treating a subject having a disease or disorder associated with cells having a SHP2 mutation, the method comprising:
administering to the subject a therapeutically effective amount of a SOS1 inhibitor.

2. The method of claim 1, wherein the SHP2 mutation induces an activated form of SHP2.

3. The method of claim 1, wherein the subject expressed the SHP2 mutation after prior treatment with a SHP2 inhibitor and further wherein the SHP2 mutation confers resistance to a SHP2 inhibitor or an allosteric SHP2 inhibitor.

4. (canceled)

5. (canceled)

6. (canceled)

7. (canceled)

8. (canceled)

9. (canceled)

10. The method of claim 1, wherein SHP2 mutation is at a position selected from the group consisting of T52, I56, G60, D61, Y⁶², Y⁶³, E69, K₇₀, A72, T73, E76, E123, E139, Y¹⁹⁷, S189, T253, Q257, L261, L262, R265, F285, N308, V428, A461, T468, P491, S₅₀₂, G503, M504, Q506, Q510, T507, and a combination thereof.

11. (canceled)

12. (canceled)

13. (canceled)

14. (canceled)

15. (canceled)

16. The method of claim 1, wherein the SOS1 inhibitor is a compound having the structure of Formula (41-I), 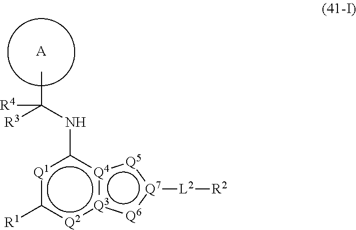
or a pharmaceutically acceptable salt, solvate, stereoisomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a} cyclopropyl, and —CN; wherein C₁₋₆ alkyl is optionally substituted with halogen, —NHR^{1a}, or —OR^{1a}, wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—, 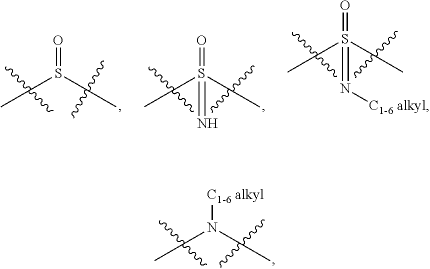
—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂),OCH₃, wherein r is 1, 2, or 3;
wherein R^{2b} is H or C₁₋₆ alkyl;
wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

17. The method of claim 1, wherein the SOS1 inhibitor is a compound having the structure of Formula (42-I), 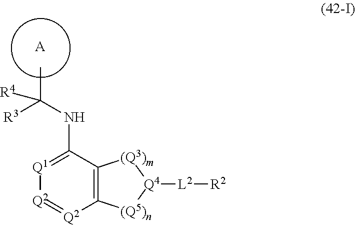
or a pharmaceutically acceptable salt, solvate, stereoisomer, prodrug, or tautomer thereof, wherein:
Q¹ is CH or N;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each RON is independently H, C₁₋₆ alkyl, or 6-10 membered aryl;
wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}, wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—, 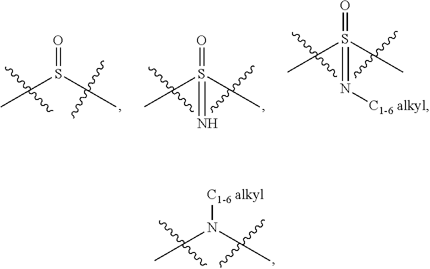
C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂),OCH₃, wherein r is 1, 2, or 3;
wherein R^{2b} is H or C₁₋₆ alkyl;
wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl;
with the proviso that when 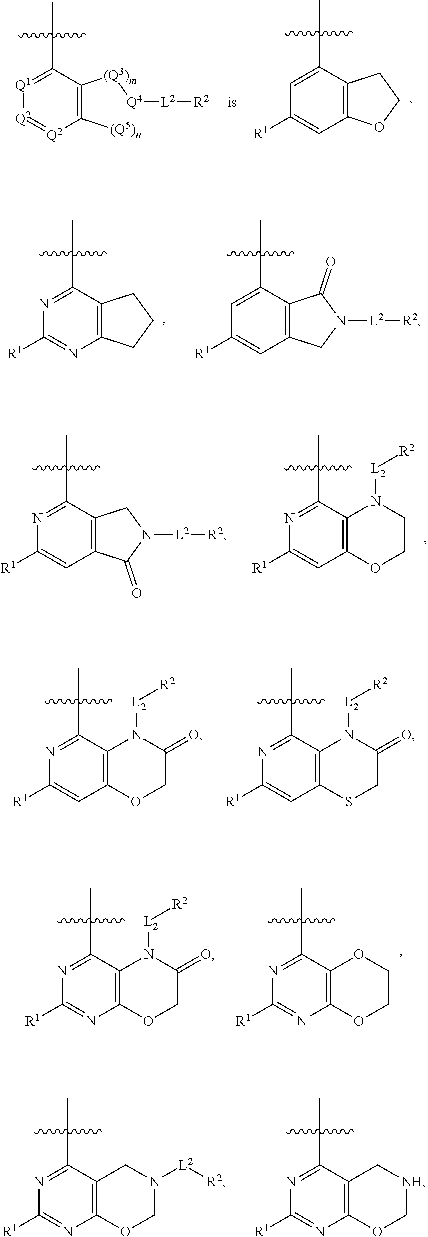 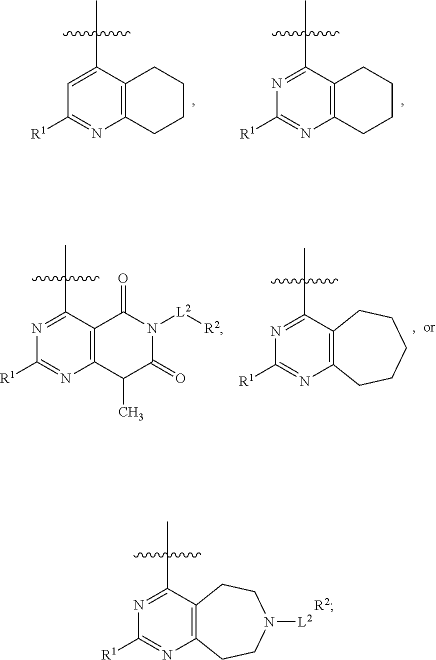
then R¹ is not H.

18. The method of claim 1, wherein the SOS1 inhibitor is a compound having the structure of Formula (48-I), 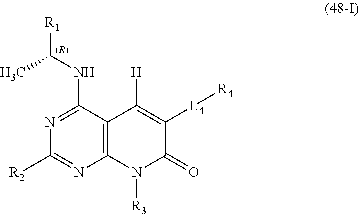
or a pharmaceutically acceptable salt, solvate, stereoisomer, prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl;
R₃ is selected from the group consisting of H, C₁₋₃ alkyl, OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, and —CN;
L₄ is selected from the group consisting of bond, —C(O)—, —C(O)O—, —C(O)NH(CH₂)ₒ—, —NH—, —S—, —S(O)₂—, 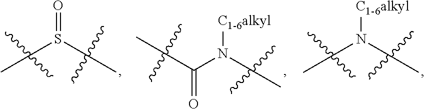
—(CH₂)ₚ—, and —O—; wherein o is 0, 1, or 2; and wherein p is a number from 1 to 6; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO) R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl, 3-7 membered heterocyclyl, 6-10 membered aryl, or 5-10 membered heteroaryl;
wherein R₄ₐ is H, C₁₋₆ alkyl, C₁₋₆ haloalkyl, —C(O)NR_{4b}R_{4c}, 3-6 membered cycloalkyl, 6-10 membered aryl optionally substituted with —OR_{4b}, —CN, 3-7 membered heterocyclyl, —(CH₂),OCH₃, or —(CH₂),OH, wherein r is 1, 2, or 3;
wherein each R_{4b} is independently H, C₁₋₆ alkyl; and
wherein each R_{4c} is independently H or C₁₋₆ alkyl.

19. The method of claim 1, wherein the SOS1 inhibitor is BI-3406, having the structure: 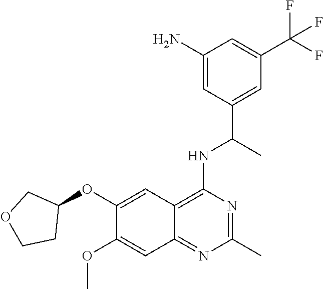 [SMILES: COc1cc2nc(C)nc(NC(C)c3cc(N)cc(C(F)(F)F)c3)c2cc1O[C@H]1CCOC1]
or a pharmaceutically acceptable salt, solvate, stereoisomer, prodrug, or tautomer thereof.

20. The method of claim 1, wherein the SOS1 inhibitor is BI-1701963 or a pharmaceutically acceptable salt, solvate, stereoisomer, prodrug, or tautomer thereof.

21. The method of claim 1, wherein the SOS1 inhibitor is BAY-293, having the structure: 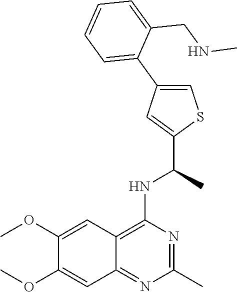
or a pharmaceutically acceptable salt, solvate, stereoisomer, prodrug, or tautomer thereof.

22. The method of claim 1, wherein the SOS1 inhibitor is SDGR5 or a pharmaceutically acceptable salt, solvate, stereoisomer, prodrug, or tautomer thereof.

23. The method of claim 1, wherein the SOS1 inhibitor is Compound SOS1-(A) (also called RMC-0331), having the structure: 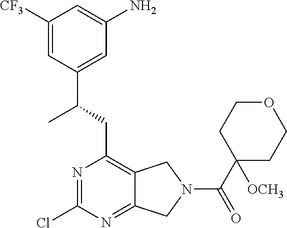 [SMILES: COC1(C(=O)N2Cc3nc(Cl)nc(C[C@H](C)c4cc(N)cc(C(F)(F)F)c4)c3C2)CCOCC1]
or a pharmaceutically acceptable salt, solvate, stereoisomer, prodrug, or tautomer thereof.

24. The method of claim 1, wherein the SOS1 inhibitor is Compound SOS1-(B) or a pharmaceutically acceptable salt, solvate, stereoisomer, prodrug, or tautomer thereof.

25. The method of claim 1 further comprising administering to the subject a therapeutically effective amount of a RAS inhibitor.

26. The method of claim 25, wherein the RAS inhibitor is selected from the group consisting of a RAS(ON) inhibitor, a RAS(OFF) inhibitor, MRTX1133 or a pharmaceutically acceptable salt, solvate, stereoisomer, prodrug, or tautomer thereof, and a combination thereof.

27. The method of claim 25, wherein the RAS inhibitor is selective for a mutation at position 12 or 13 of a RAS protein.

28. The method of claim 25, wherein the RAS inhibitor is a RAS(ON) inhibitor.

29. The method of claim 28, wherein the RAS(ON) inhibitor is an inhibitor selective for RAS G12C, RAS G13D, or RAS G12D.

30. The method of claim 28, wherein the RAS(ON) inhibitor is a RAS(ON) MULTI inhibitor.

31. (canceled)

32. (canceled)

33. (canceled)

34. (canceled)

35. (canceled)

36. (canceled)

37. (canceled)

38. (canceled)

39. (canceled)

40. The method of claim 28, wherein the RAS(ON) inhibitor is selected from the group consisting of RAS-(D), RAS-(E), or a pharmaceutically acceptable salt, solvate, stereoisomer, prodrug, or tautomer thereof, and a combination thereof.

41. The method of claim 25, wherein the RAS inhibitor is a RAS(OFF) inhibitor.

42. The method of claim 41, wherein the RAS(OFF) inhibitor selectively targets RAS G12C.

43. The method of claim 42, wherein the RAS(OFF) inhibitor is selected from the group consisting of sotorasib (AMG 510), adagrasib (MRTX849), MRTX1257, JNJ-74699157 (ARS-3248), LY3537982, ARS-853, ARS-1620, GDC-6036, BPI-421286, JDQ443, and JAB-21000, or a pharmaceutically acceptable salt, solvate, stereoisomer, prodrug, or tautomer thereof.

44. The method of claim 25, wherein the RAS inhibitor selectively targets RAS G12D.

45. The method of claim 44, wherein the RAS inhibitor is MRTX1133 or a pharmaceutically acceptable salt, solvate, stereoisomer, prodrug, or tautomer thereof.

46. The method of claim 1 further comprising administering to the subject a therapeutically effective amount of a MEK inhibitor.

47. (canceled)

48. The method of claim 1 further comprising administering to the subject a therapeutically effective amount of a CDK4/6 inhibitor.

49. (canceled)

50. The method of claim 1 further comprising administering to the subject a therapeutically effective amount of a PD-1 inhibitor.

51. (canceled)

52. The method of claim 1, wherein the disease or disorder is selected from the group consisting of tumors of hematopoietic and lymphoid system; a myeloproliferative syndrome; a myelodysplastic syndrome; leukemia; acute myeloid leukemia; acute B-lymphoblastic leukemia-lymphoma, juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer; thyroid cancer; endometrial cancer; cancer of the biliary tract; soft tissue cancer; ovarian cancer; central nervous system cancer; stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma.

53. (canceled)

54. (canceled)

55. (canceled)

56. (canceled)

57. (canceled)

58. (canceled)

59. (canceled)

60. The method of claim 1, wherein the disease or disorder is a RASopathy.

61. (canceled)

62. (canceled)

63. (canceled)

64. The method of claim 1, further comprising performing a diagnostic test to determine whether the subject has a SHP2 mutation that induces an activated form of SHP2.

65. The method of claim 25, wherein the RAS inhibitor targets a wild-type RAS protein.

66. The method of claim 25, wherein the RAS inhibitor targets a RAS protein mutation.

67. (canceled)

68. (canceled)

69. (canceled)

70. The method of claim 25, wherein the RAS protein is KRAS.
